SEQUENCE LISTING (1 sequence)

SEQ ID NO: 1 - protein, 847 aa, Homo sapiens
MSLWGLVSKMPPEKVQRLYVDFPQHLRHLLGDWLESQPWEFLVGSDAFCCNLASALLSDTVQHLQASVGEQGEGSTILQHISTLESIYQRDPLKLVATFRQILQGEKKAVMEQFRHLPMPFHWKQEELKFKTGLRRLQHRVGEIHLLREALQKGAEAGQVSLHSLIETPANGTGPSEALAMLLQETTGELEAAKALVLKRIQIWKRQQQLAGNGAPFEESLAPLQERCESLVDIYSQLQQEVGAAGGELEPKTRASLTGRLDEVLRTLVTSCFLVEKQPPQVLKTQTKFQAGVRFLLGLRFLGAPAKPPLVRADMVTEKQARELSVPQGPGAGAESTGEIINNTVPLENSIPGNCCSALFKNLLLKKIKRCERKGTESVTEEKCAVLFSASFTLGPGKLPIQLQALSLPLVVIVHGNQDNNAKATILWDNAFSEMDRVPFVVAERVPWEKMCETLNLKFMAEVGTNRGLLPEHFLFLAQKIFNDNSLSMEAFQHRSVSWSQFNKEILLGRGFTFWQWFDGVLDLTKRCLRSYWSDRLIIGFISKQYVTSLLLNEPDGTFLLRFSDSEIGGITIAHVIRGQDGSPQIENIQPFSAKDLSIRSLGDRIRDLAQLKNLYPKKPKDEAFRSHYKPEQMGKDGRGYVPATIKMTVERDQPLPTPELQMPTMVPSYDLGMAPDSSMSMQLGPDMVPQVYPPHSHSIPPYQGLSPEESVNVLSAFQEPHLQMPPSLGQMSLPFDQPHPQGLLPCQPQEHAVSSPDPLLCSDVTMVEDSCLSQPVTAFPQGTWIGEDIFPPLLPPTEQDLTKLLLEGQGESGGGSLGAQPLLQPSHYGQSGISMSHMDLRANPSW

(19) United States
(12) Patent Application Publication
(10) Pub. No.: US 2026/0124309 A1
(43) Pub. Date: May 7, 2026

(54) STAT6 DEGRADERS AND USES THEREOF

(71) Applicant: Kymera Therapeutics, Inc., Watertown, MA (US)

(72) Inventors: Bin Yang, Watertown, MA (US); Bruce C. Follows, Watertown, MA (US); Huijun Dong, Watertown, MA (US); Xiao Zhu, Watertown, MA (US); Matthew M. Weiss, Watertown, MA (US); Xiaozhang Zheng, Watertown, MA (US); Xin Huang, Watertown, MA (US); Lijing Su, Watertown, MA (US); Nello Mainolfi, Watertown, MA (US); Thijs Beuming, Cambridge, MA (US); Xue Fei, Watertown, MA (US); Philip Collier, Watertown, MA (US); Yi Zhang, Watertown, MA (US)

(21) Appl. No.: 19/164,719

(22) PCT Filed: Aug. 29, 2024
(86) PCT No.: PCT/US2024/044544

§ 371 Date: Sep. 12, 2025

Related U.S. Application Data
(60) Provisional application No. 63/579,812, filed on Aug. 30, 2023.
(60) Provisional application No. 63/617,359, filed on Jan. 3, 2024.
(60) Provisional application No. 63/649,863, filed on May 20, 2024.
(60) Provisional application No. 63/678,903, filed on Aug. 2, 2024.

(51) Int. Cl. A61K 47/55; A61P 35/00

(52) U.S. Cl. CPC A61K 47/55; A61P 35/00

(57) ABSTRACT

The present invention provides compounds, compositions thereof, and methods of using the same.

### CROSS REFERENCE TO RELATED APPLICATIONS

This application claims the benefit of priority from U.S. Provisional Appl. No. 63/579,812, filed Aug. 30, 2023, U.S. Provisional Appl. No. 63/617,359, filed Jan. 3, 2024, U.S. Provisional Appl. No. 63/649,863, filed May 20, 2024, and U.S. Provisional Appl. No. 63/678,903, filed Aug. 2, 2024.

### TECHNICAL FIELD OF THE INVENTION

The present invention relates to compounds and methods useful for the modulation of signal transducer and activator of transcription 6 (“STAT6”) via ubiquitination and/or degradation by compounds according to the present invention. The invention also provides pharmaceutically acceptable compositions comprising compounds of the present invention and methods of using said compositions in the treatment of various disorders.

### BACKGROUND OF THE INVENTION

Ubiquitin-Proteasome Pathway (UPP) or Ubiquitin-Proteasome System (UPS) is a critical pathway that regulates key regulator proteins and degrades misfolded or abnormal proteins. UPP is central to multiple cellular processes, and if defective or imbalanced, it leads to pathogenesis of a variety of diseases. The covalent attachment of ubiquitin to specific protein substrates is achieved through the action of E3 ubiquitin ligases.

The UPP is used to induce selective protein degradation, including use of fusion proteins to artificially ubiquitinate target proteins and synthetic small-molecule probes to induce proteasome-dependent degradation. Bifunctional compounds composed of a target protein-binding ligand and an E3 ubiquitin ligase ligand, induced proteasome-mediated degradation of selected proteins via their recruitment to E3 ubiquitin ligase and subsequent ubiquitination. These drug-like molecules offer the possibility of temporal control over protein expression. Such compounds are capable of inducing the inactivation of a protein of interest upon addition to cells or administration to an animal or human, and could be useful as biochemical reagents and lead to a new paradigm for the treatment of diseases by removing pathogenic or oncogenic proteins (Crews, C., Chemistry & Biology, 2010, 17(6):551-555: Schnnekloth, J. S. Jr., Chembiochem, 2005, 6(1):40-46).

Signal transducer and activator of transcription 6 (STAT6 or Interleukin-4-Stat/IL4-STAT) is an undruggable transcription factor belonging to the structurally conserved Signal Transducer and Activator of Transcription (STAT) family of proteins (STAT1 through STAT6). Activation of STAT6, like other STAT proteins, is triggered upon binding of hormones, immunomodulatory cytokines or growth factors to specific receptors on the cell surface. Once activated, the phosphorylation of a C-terminal tyrosine residue occurs, leading to translocation and transmission of signals from the cytosol to the nucleus, resulting in activation of gene expression.

STAT6 is implicated in driving Type 2 immunity, allergies. It may participate in IL-4/IL-13-mediated allergic reaction, and play a vital role in the differentiation of T-helper type 2 (Th2) cells (Hebenstreit et al. “Signaling mechanisms, interaction partners, and target genes of STAT6.” Cytokine & growth factor reviews 17.3 (2006): 173-188; Chapoval et al. “Regulation of the T helper cell type 2 (Th2)/T regulatory cell (Treg) balance by IL-4 and STAT6.” Journal of leukocyte biology 87.6 (2010): 1011-1018). STAT6 is a key node primarily activated in the Janus Kinase (JAK) pathway by inflammatory cytokines, interleukin-4 (IL4) and interleukin-13 (IL13) and their cognate receptors, which are produced by Th2 cells, mast cells and basophils. Human STAT6 mutations have been associated with severe allergies such as asthma and eczema (Goenka and Kaplan. “Transcriptional regulation by STAT6.” Immunologic research 50.1 (2011): 87-96.). There is a need to discover and develop STAT6 drugs, for example to treat allergic/inflammatory diseases and cancers (Glosson et al. “Wheezing and itching: The requirement for STAT proteins in allergic inflammation.” Jak-Stat 1.1 (2012): 3-15: Loh et al. “Signal transducer and activator of transcription (STATs) proteins in cancer and inflammation: functions and therapeutic implication.” Frontiers in oncology 9 (2019): 48). As such, small molecule compounds that leverage E3 ligase mediated protein degradation to target disease-associated proteins such as STAT6 hold promise as therapeutic agents.

### SUMMARY OF THE INVENTION

The present application relates to novel bifunctional compounds, which function to recruit STAT6 protein to E3 ubiquitin ligase for degradation, and methods of preparation and uses thereof. In particular, the present disclosure provides bifunctional compounds, which find utility as modulators of targeted ubiquitination of STAT6 protein, which are then degraded and/or otherwise inhibited by the bifunctional compounds as described herein. Also provided are monovalent compounds, which find utility as inducers of targeted ubiquitination of STAT6 protein, which are then degraded and/or otherwise inhibited by the monovalent compounds as described herein. An advantage of the compounds provided herein is that a broad range of pharmacological activities is possible, consistent with the degradation/inhibition of STAT6 protein. In addition, the description provides methods of using an effective amount of the compounds as described herein for the treatment or amelioration of a disease condition, such as inflammatory disorders.

The present application further relates to targeted degradation of STAT6 protein through the use of bifunctional molecules, including bifunctional molecules that link a cereblon or VHL binding moiety to a ligand that binds STAT6 protein.

It has now been found that compounds of this invention, and pharmaceutically acceptable compositions thereof, are effective as degraders of STAT6 protein. Such compounds have the general formula I:

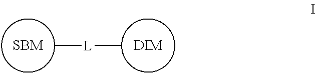

or a pharmaceutically acceptable salt thereof, wherein each variable is as defined and described herein.

Compounds of the present invention, and pharmaceutically acceptable compositions thereof, are useful for treating a variety of diseases, disorders or conditions, associated with regulation of signaling pathways implicating STAT6 protein. Such diseases, disorders, or conditions include those described herein.

Compounds provided by this invention are also useful for the study of STAT6 protein in biological and pathological phenomena: the study of intracellular signal transduction pathways occurring in bodily tissues; and the comparative evaluation of new STAT6 inhibitors or STAT6 degraders or other regulators of cell cycling, metastasis, angiogenesis, and immune cell evasion, in vitro or in vivo.

### BRIEF DESCRIPTION OF THE DRAWINGS

FIG. 1 shows rapid and highly potent (A) and selective (B) STAT6 degradation by I-1 in human PBMC.

FIG. 2 shows superior IL-4 and IL-13 TARC (A) and CD23 (B) inhibition potencies achieved by STAT6 degrader I-1 versus dupilumab (C) in human PBMC. Dupilumab data: LeFloc'h et al. Allergy 2020, 75(5):1188-1204.

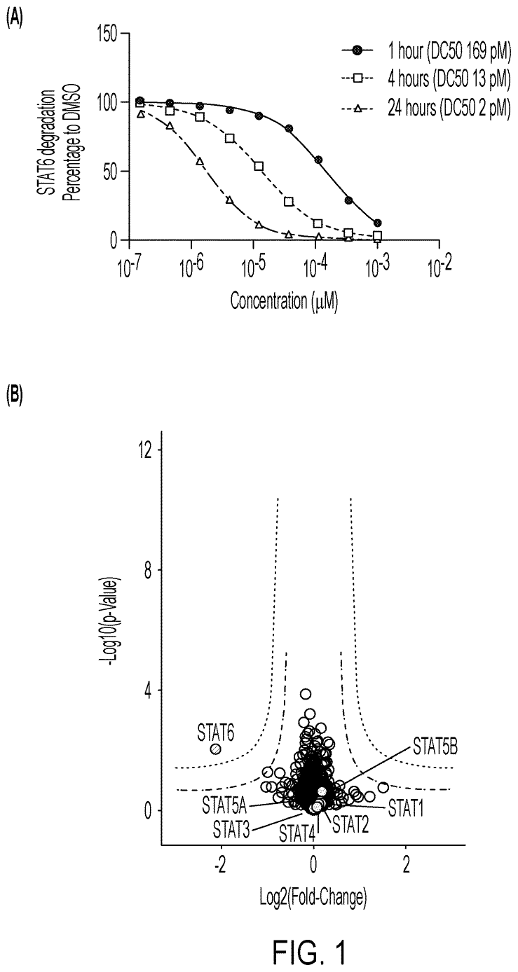

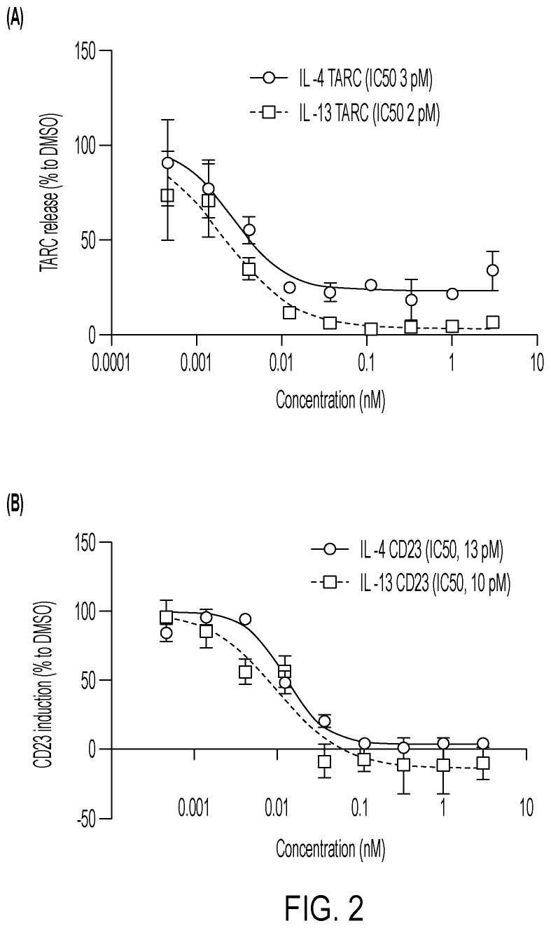

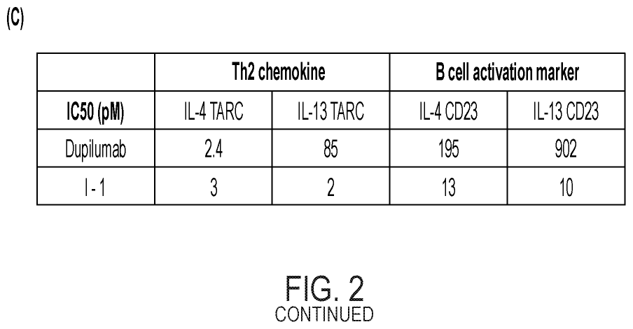

### DETAILED DESCRIPTION OF CERTAIN EMBODIMENTS

### 1. General Description of Certain Embodiments of the Invention

Compounds of the present invention, and compositions thereof, are useful as degraders and/or inhibitors of STAT6 protein. In some embodiments, a provided compound degrades and/or inhibits STAT6.

In certain embodiments, the present invention provides a compound of formula I:

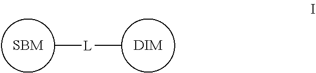

or a pharmaceutically acceptable salt thereof, wherein:
SBM is a STAT6 binding moiety capable of binding to STAT6 protein as defined and described herein;
L is a bivalent moiety that connects SBM to DIM as defined and described herein; and
DIM is a degradation inducing moiety selected from an E3 ubiquitin ligase binding moiety (LBM), lysine mimetic, and hydrogen as defined and described herein.

### 2. Compounds and Definitions

Compounds of the present invention include those described generally herein, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. In some embodiments, a carbocyclic ring may be a 5-12 membered bicyclic, bridged bicyclic, or spirocyclic ring. A carbocyclic ring may include one or more oxo (═O) or thioxo (═S) substituent. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bridged bicyclics include:

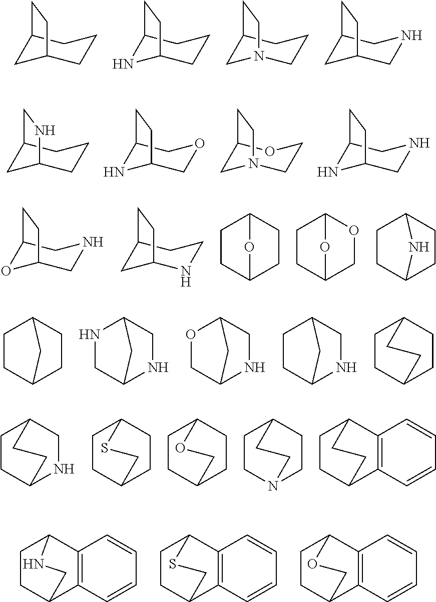

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁻ (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

As used herein, the term “cyclopropylenyl” refers to a bivalent cyclopropyl group of the following structure:

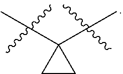 [SMILES: *C1(*)CC1]

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like. The term “arylenyl” refers to bivalent aryl groups (e.g., phenylenyl).

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinvl, tetrahydroisoquinolinyl, and pyrido[2,3-b]-1,4-oxazin-3(4H)-one. A heteroaryl group may be monocyclic, bicyclic, bridged bicyclic, or spirocyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted. The term “heteroarylenyl” refers to bivalent heteroaryl groups (e.g., pyridylenyl).

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical.” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. In some embodiments, a heterocyclic ring may be a 5-12 membered bicyclic, bridged bicyclic, or spirocyclic ring. A heterocyclic ring may include one or more oxo (═O) or thioxo (═S) substituent. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the disclosure may contain “substituted” moieties. In general, the term “substituted” means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable.” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄₀R^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄S R^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —(CH₂)₀₋₄P(O)₂R^{∘}; —(CH₂)O—₄P(O)R^{∘}₂; —(CH₂)₀₋₄OP(O)R^{∘}₂; —(CH₂)₀₋₄OP(O)(OR^{∘})₂; SiR^{∘}₃; —(C₁—₄ straight or branched alkylene)O—N(R^{∘})₂; or —(C₁—₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 3-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R⁵⁷⁴, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include; —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of Rt are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. In some embodiments, the provided compounds are purified in salt form for convenience and/or ease of purification, e.g., using an acidic or basic mobile phase during chromatography. Salts forms of the provided compounds formed during chromotagraphic purification are contemplated herein (e.g., diammonium salts) and are readily apparent to those having skill in the art.

Unless otherwise stated, structures depicted herein are also meant to include all isomeric (e.g., enantiomeric, diastereomeric, and geometric (or conformational)) forms of the structure; for example, the R and S configurations for each asymmetric center. Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C or ¹⁴C-enriched carbon are within the scope of this invention. Such compounds are useful, for example, as analytical tools, as probes in biological assays, or as therapeutic agents in accordance with the present invention

As used herein, the term “about” refers to within 20% of a given value. In some embodiments, the term “about” refers to within 20%, 19%, 18%, 17%, 16%, 15%, 14%, 13%, 12%, 11%, 10%, 9%, 8%, 7%, 6%, 5%, 4%, 3%, 2%, or 1% of a given value.

As used herein, the term “provided compound” refers to any genus, subgenus, and/or species set forth herein.

As used herein, the term “inhibitor” is defined as a compound that binds to and/or inhibits STAT6 protein with measurable affinity. In certain embodiments, an inhibitor has an IC₅₀ and/or binding constant of less than about 50 μM, less than about 1 μM, less than about 500 nM, less than about 100 nM, less than about 10 nM, or less than about 1 nM.

As used herein, the term “degrader” is defined as a heterobifunctional compound that binds to and/or inhibits both STAT6 protein and an E3 ligase with measurable affinity resulting in the ubiquitination and subsequent degradation of the STAT6 protein. In certain embodiments, a degrader has an DC₅₀ of less than about 50 μM, less than about 1 μM, less than about 500 nM, less than about 100 nM, less than about 10 nM, or less than about 1 nM. As used herein, the term “monovalent” refers to a degrader compound without an appended E3 ligase binding moiety.

A compound of the present invention may be tethered to a detectable moiety. It will be appreciated that such compounds are useful as imaging agents. One of ordinary skill in the art will recognize that a detectable moiety may be attached to a provided compound via a suitable substituent. As used herein, the term “suitable substituent” refers to a moiety that is capable of covalent attachment to a detectable moiety. Such moieties are well known to one of ordinary skill in the art and include groups containing, e.g., a carboxylate moiety, an amino moiety, a thiol moiety, or a hydroxyl moiety, to name but a few. It will be appreciated that such moieties may be directly attached to a provided compound or via a tethering group, such as a bivalent saturated or unsaturated hydrocarbon chain. In some embodiments, such moieties may be attached via click chemistry. In some embodiments, such moieties may be attached via a 1,3-cycloaddition of an azide with an alkyne, optionally in the presence of a copper catalyst. Methods of using click chemistry are known in the art and include those described by Rostovtsev et al., Angew. Chem. Int. Ed. 2002, 41, 2596-99 and Sun et al., Bioconjugate Chem., 2006, 17, 52-57.

As used herein, the term “detectable moiety” is used interchangeably with the term “label” and relates to any moiety capable of being detected, e.g., primary labels and secondary labels. Primary labels, such as radioisotopes (e.g., tritium, ³²P, ³³P, ³⁵S, or ¹⁴C), mass-tags, and fluorescent labels are signal generating reporter groups which can be detected without further modifications. Detectable moieties also include luminescent and phosphorescent groups.

The term “secondary label” as used herein refers to moieties such as biotin and various protein antigens that require the presence of a second intermediate for production of a detectable signal. For biotin, the secondary intermediate may include streptavidin-enzyme conjugates. For antigen labels, secondary intermediates may include antibody-enzyme conjugates. Some fluorescent groups act as secondary labels because they transfer energy to another group in the process of nonradiative fluorescent resonance energy transfer (FRET), and the second group produces the detected signal.

The terms “fluorescent label”, “fluorescent dye”, and “fluorophore” as used herein refer to moieties that absorb light energy at a defined excitation wavelength and emit light energy at a different wavelength. Examples of fluorescent labels include, but are not limited to: Alexa Fluor dyes (Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), AMCA, AMCA-S, BODIPY dyes (BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), Carboxyrhodamine 6G, carboxy-X-rhodamine (ROX), Cascade Blue, Cascade Yellow, Coumarin 343, Cyanine dyes (Cy3, Cy5, Cy3.5, Cy5.5), Dansyl, Dapoxyl, Dialkylaminocoumarin, 4′,5′-Dichloro-2′,7′-dimethoxy-fluorescein, DM-NERF, Eosin, Erythrosin, Fluorescein, FAM, Hydroxycoumarin, IRDyes (IRD40, IRD 700, IRD 800), JOE, Lissamine rhodamine B, Marina Blue, Methoxycoumarin, Naphthofluorescein, Oregon Green 488, Oregon Green 500, Oregon Green 514, Pacific Blue. PyMPO, Pyrene, Rhodamine B, Rhodamine 6G. Rhodamine Green, Rhodamine Red, Rhodol Green, 2′,4′,5′,7′-Tetra-bromosulfone-fluorescein, Tetramethyl-rhodamine (TMR), Carboxytetramethylrhodamine (TAMRA), Texas Red, Texas Red-X.

The term “mass-tag” as used herein refers to any moiety that is capable of being uniquely detected by virtue of its mass using mass spectrometry (MS) detection techniques. Examples of mass-tags include electrophore release tags such as N-[3-[4′-[(p-Methoxytetrafluorobenzyl)oxy]phenyl]-3-methylglyceronyl]isonipecotic Acid, 4′-[2,3,5,6-Tetrafluoro-4-(pentafluorophenoxyl)]methyl acetophenone, and their derivatives. The synthesis and utility of these mass-tags is described in U.S. Pat. Nos. 4,650,750, 4,709,016, 5,360,8191, 5,516,931, 5,602,273, 5,604,104, 5,610,020, and 5,650,270. Other examples of mass-tags include, but are not limited to, nucleotides, dideoxynucleotides, oligonucleotides of varying length and base composition, oligopeptides, oligosaccharides, and other synthetic polymers of varying length and monomer composition. A large variety of organic molecules, both neutral and charged (biomolecules or synthetic compounds) of an appropriate mass range (100-2000 Daltons) may also be used as mass-tags.

The terms “measurable affinity” and “measurably inhibit,” as used herein, means a measurable change in STAT6 protein activity between a sample comprising a compound of the present invention, or composition thereof, and STAT6 protein, and an equivalent sample comprising STAT6 protein, in the absence of said compound, or composition thereof.

As used herein, the term “reference” describes a standard or control relative to which a comparison is performed. In some embodiments, a “reference” sample or subject is one that is sufficiently similar to a particular sample or subject of interest to permit a relevant comparison. For example, in some embodiments, an agent, animal, individual, population, sample, sequence or value of interest is compared with a reference or control agent, animal individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment Those skilled in the art will appreciate when sufficient similarities are present to justify reliance on and/or comparison to a particular possible reference or control.

### 3. Description of Exemplary Embodiments

In certain embodiments, the present invention provides a compound of formula I:

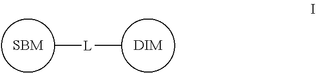

or a pharmaceutically acceptable salt thereof, wherein:
SBM is a STAT6 binding moiety capable of binding to STAT6 protein as defined and described herein;
L is a bivalent moiety that connects SBM to DIM as defined and described herein; and
DIM is a degradation inducing moiety selected from an E3 ubiquitin ligase binding moiety (LBM), lysine mimetic, and hydrogen as defined and described herein.

In some embodiments, the compound of formula I specifically affects, as its primary mechanism of action, the degradation of STAT6.

### STAT6 Binding Moiety (SBM)

In certain embodiments, the present invention provides a compound of formula I, wherein SBM is a STAT6 binding moiety of formula I-a:

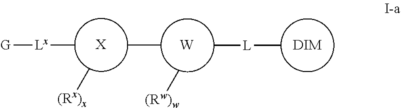

or a pharmaceutically acceptable salt thereof, wherein L and DIM are as defined and described herein, and wherein:
Ring W is a 9-membered bicyclic heteroaryl with 1-4 heteroatoms independently selected from nitrogen, oxygen, and sulfur;
Ring X is 6-membered saturated or partially unsaturated carbocyclylenyl or heterocyclylenyl with 1-3 heteroatoms independently selected from nitrogen, oxygen, and sulfur;
G is hydrogen, halogen, or

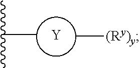

Ring Y is a 3- to 6-membered saturated or partially unsaturated carbocyclyl, or 4- to 6-membered monocyclic saturated or partially unsaturated heterocyclyl or heteroaryl ring with 1-4 heteroatoms independently selected from nitrogen, oxygen, or sulfur:
R_{w}, R^{x}, and R^{y} are independently selected from hydrogen, R^{A}, halogen, —CN, —NO₂, —OR, —SR, —NR₂, —SiR₃, —S(O)R, —S(O)₂R, —S(O)(NR)R, —S(O)₂NR₂, —S(O)R, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R;
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom or adjacent atoms are optionally taken together with their intervening atoms to form a 3-7 membered saturated or partially unsaturated ring having 0-3 heteroatoms, in addition to the atom or adjacent atoms to which they are attached, independently selected from nitrogen, oxygen, and sulfur;
L^{x} is a covalent bond or a C₁₋₅ bivalent straight or branched saturated or unsaturated hydrocarbon chain wherein 1-2 methylene units of the chain are independently and optionally replaced with —O—, —C(O)—, —C(S)—, —CR₂—, —NR—, —S—, —S(O)—, or —S(O)₂—; and
each of w, x, and v are independently 0, 1, 2, 3, or 4.

In certain embodiments, the present invention provides a compound of formula I, wherein SBM is a STAT6 binding moiety of formula I-a′:

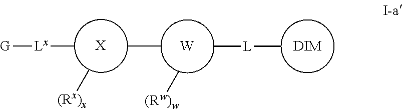

or a pharmaceutically acceptable salt thereof, wherein L and DIM are as defined and described herein, and wherein:
Ring W is a 9-membered bicyclic heteroaryl with 1-4 heteroatoms independently selected from nitrogen, oxygen, and sulfur;
Ring X is a 6-membered saturated or partially unsaturated carbocyclylenyl or heterocyclylenyl with 1-3 heteroatoms independently selected from nitrogen, oxygen, and sulfur;
G is hydrogen, halogen, or

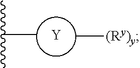

Ring Y is a 3- to 6-membered saturated or partially unsaturated carbocyclyl, or 4- to 6-membered monocyclic saturated or partially unsaturated heterocyclyl or heteroaryl ring with 1-4 heteroatoms independently selected from nitrogen, oxygen, or sulfur;
R^{w}, R^{x}, and R^{y} are independently selected from hydrogen, R^{A}, halogen, —CN, —NO₂, —OR, —SR, —NR₂, —SiR₃, —S(O)R, —S(O)₂R, —S(O)(NR)R, —S(O)₂NR₂, —S(O)R, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R;
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-10 membered monocyclic or bicyclic heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-10 membered heteroaryl ring having 1-4 heteroatoms independently selected from nitrogen, oxygen, and sulfur, or:
   two R groups on the same atom or adjacent atoms are optionally taken together with their intervening atoms to form a 3-10 membered saturated or partially unsaturated monocyclic, bicyclic, bridged bicyclic, or spirocyclic ring having 0-3 heteroatoms, in addition to the atom or adjacent atoms to which they are attached, independently selected from nitrogen, oxygen, and sulfur;
L^{x} is a covalent bond or a C₁₋₅ bivalent straight or branched saturated or unsaturated hydrocarbon chain wherein 1-2 methylene units of the chain are independently and optionally replaced with —O—, —C(O)—, —C(S)—, —NR—, —S—, —S(O)—, or —S(O)₂—; and
each of w, x, and y are independently 0, 1, 2, 3, or 4.

In certain embodiments, the present invention provides a compound of formula I, wherein SBM is a STAT6 binding moiety of formula I-a″:

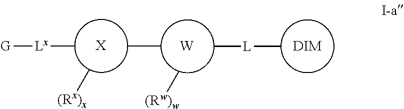

or a pharmaceutically acceptable salt thereof, wherein L and DIM are as defined and described herein, and wherein:
Ring W is a 9-membered bicyclic heteroaryl with 1-4 heteroatoms independently selected from nitrogen, oxygen, and sulfur;
Ring X is a 6-membered saturated or partially unsaturated carbocyclylenyl or heterocyclylenyl with 1-3 heteroatoms independently selected from nitrogen, oxygen, and sulfur;
G is hydrogen, halogen, or

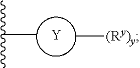

Ring Y is a 3- to 6-membered saturated or partially unsaturated carbocyclyl, or 4- to 6-membered monocyclic saturated or partially unsaturated heterocyclyl or heteroaryl ring with 1-4 heteroatoms independently selected from nitrogen, oxygen, or sulfur:
R^{w}, R^{x}, and R^{y} are independently selected from hydrogen, R^{A}, halogen, —CN, —NO₂, —OR, —SR, —NR₂, —SiR₃, —S(O)R, —S(O)₂R, —S(O)(NR)R, —S(O)₂NR₂, —S(O)R, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R;
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-10 membered monocyclic or bicyclic heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-10 membered heteroaryl ring having 1-4 heteroatoms independently selected from nitrogen, oxygen, and sulfur, or:
   two R groups on the same atom or adjacent atoms are optionally taken together with their intervening atoms to form a 3-10 membered saturated or partially unsaturated monocyclic, bicyclic, bridged bicyclic, or spirocyclic ring having 0-3 heteroatoms, in addition to the atom or adjacent atoms to which they are attached, independently selected from nitrogen, oxygen, and sulfur;
L^{x} is a covalent bond or an optionally substituted C₁₋₅; bivalent straight or branched saturated or unsaturated hydrocarbon chain wherein 1-2 methylene units of the chain are independently and optionally replaced with —O—, —C(O)—, —C(S)—, —CR₂—, —NR—, —S—, —S(O)—, or —S(O)₂—; and
each of w, x, and y are independently 0, 1, 2, 3, or 4.

In some embodiments, the present invention provides a compound of formula I-a′″:

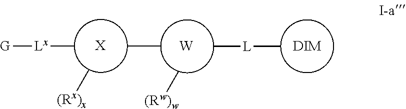

or a pharmaceutically acceptable salt thereof, wherein L and DIM are as defined and described herein, wherein:
Ring W is a 9-membered bicyclic heteroaryl with 1-4 heteroatoms independently selected from nitrogen, oxygen, and sulfur;
Ring X is a 6-membered saturated or partially unsaturated carbocyclylenyl or heterocyclylenyl with 1-3 heteroatoms independently selected from nitrogen, oxygen, and sulfur:
G is hydrogen, halogen, or

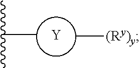

Ring Y is a 3- to 6-membered saturated or partially unsaturated carbocyclyl, or 4- to 6-membered monocyclic saturated or partially unsaturated heterocyclyl or heteroaryl ring with 1-4 heteroatoms independently selected from nitrogen, oxygen, or sulfur;
R^{x} and R^{y} are independently selected from hydrogen, R^{A}, halogen, —CN, —NO₂, —OR, —SR, —NR₂, —SiR₃, —S(O)R, —S(O)₂R, —S(O)(NR)R, —S(O)₂NR₂, —S(O)R, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R;
each R^{w} is independently selected from hydrogen, R^{A′}, R^{B′}, halogen, —CN, —NO₂, —OR, —SR, —NR₂, —SiR₃, —S(O)R, —S(O)₂R, —S(O)(NR)R, —S(O)₂NR₂, —S(O)R, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R;
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R^{A′} is independently R^{A} or an 8-10 membered monocyclic or bicyclic aryl or heteroaryl with 1-4 heteroatoms independently selected from nitrogen, oxygen and sulfur:
each R^{B′} is independently -L^{B}-Cy^{B1}-H or -L^{B}-Cy^{B1}-Cy^{B2};
each L^{B} is independently a covalent bond or a C₁₋₃ bivalent straight or branched saturated or unsaturated hydrocarbon chain wherein 1-2 methylene units of the chain are independently and optionally replaced with —O—, —C(O)—, —C(S)—, —CR₂—, —CF₂—, —CRF—, —CR(OR)—, —NR—, —S—, —S(O)—, —S(O)₂— —S(O)(NR)— or —CR═CR—;
each Cy^{B1} is independently an optionally substituted ring selected from phenylenyl, a 3-7 membered saturated or partially unsaturated carbocyclylenyl or heterocyclylenyl having 1-4 heteroatoms independently selected from nitrogen, oxygen, and sulfur, and a 5-10 membered monocyclic or bicyclic arylenyl or heteroarylenyl having 1-4 heteroatoms independently selected from nitrogen, oxygen, and sulfur;
each Cy^{B2} is independently an optionally ring selected from phenyl, a 3-7 membered saturated or partially unsaturated carbocyclic or heterocyclic ring having 1-2 heteroatoms independently selected from nitrogen, oxygen, and sulfur, and a 5-10 membered monocyclic or bicyclic aryl or heteroaryl with 1-4 heteroatoms independently selected from nitrogen, oxygen and sulfur;
each R is independently hydrogen, or an optionally substituted group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom or adjacent atoms are optionally taken together with their intervening atoms to form a 3-7 membered saturated or partially unsaturated ring having 0-3 heteroatoms, in addition to the atom or adjacent atoms to which they are attached, independently selected from nitrogen, oxygen, and sulfur:
L^{x} is a covalent bond or an optionally substituted C s bivalent straight or branched saturated or unsaturated hydrocarbon chain wherein 1-2 methylene units of the chain are independently and optionally replaced with —O—, —C(O)—, —C(S)—, —CR₂—, —NR—, —S—, —S(O)—, or —S(O)₂—; and
each of w, x, and y are independently 0, 1, 2, 3, or 4.

In some embodiments, the present invention provides a compound of formula I-a″″:

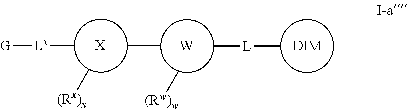

or a pharmaceutically acceptable salt thereof, wherein L and DIM are as defined and described herein, wherein:
Ring W is a 9-membered bicyclic heteroaryl with 1-4 heteroatoms independently selected from nitrogen, oxygen, and sulfur;
Ring X is a 6-membered saturated or partially unsaturated carbocyclylenyl or heterocyclylenyl with 1-3 heteroatoms independently selected from nitrogen, oxygen, and sulfur;
G is hydrogen, halogen, or

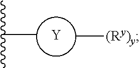

Ring Y is a 3- to 6-membered saturated or partially unsaturated carbocyclyl, or 4- to 6-membered monocyclic saturated or partially unsaturated heterocyclyl or heteroaryl ring with 1-4 heteroatoms independently selected from nitrogen, oxygen, or sulfur;
R^{x} and R^{Y} are independently selected from hydrogen, R^{A}, halogen, —CN, —NO₂, —OR, —SR, —NR₂, —SiR₃, —S(O)R, —S(O)₂R, —S(O)(NR)R, —S(O)₂NR₂, —S(O)R, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R;
each R^{w} is independently selected from hydrogen, R^{A}. R^{B′}, halogen, —CN, —NO₂, —OR, —SR, —NR₂, —SiR₃, —S(O)R, —S(O)₂R, —S(O)(NR)R, —S(O)₂NR₂, —S(O)R, —C(O)R, —C(S)R, —C(NR)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R;
each R^{A} is independently an optionally substituted group selected from C₁₋₆ aliphatic, phenyl, naphthalenyl, a 3-7 membered saturated or partially unsaturated carbocyclic or heterocyclic ring having 1-2 heteroatoms independently selected from nitrogen, oxygen, and sulfur, and a 5-10 membered monocyclic or bicyclic aryl or heteroaryl ring having 1-4 heteroatoms independently selected from nitrogen, oxygen, and sulfur, or a 7-11 membered bicyclic heterocyclic ring having 1-4 heteroatoms independently selected from nitrogen, oxygen, and sulfur,
each R^{B′} is independently -L^{B}-Cy^{B1}-H or -L^{B}-Cy^{B1}-Cy^{B2};
each L^{B} is independently a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(NR)R—, —CR₂—, —CF₂—, —CRF—, —CR(OR)—, —NR—, —S—, —S(O)—, —S(O)₂— —S(O)(NR)— or —CR═CR—;
each Cy^{B1} is independently an optionally substituted ring selected from phenylenyl, a 3-10 membered saturated or partially unsaturated monocyclic or bicyclic carbocyclylenyl or heterocyclylenyl having 1-4 heteroatoms independently selected from nitrogen, oxygen, and sulfur, and a 5-10 membered monocyclic or bicyclic arylenyl or heteroarylenyl having 1-4 heteroatoms independently selected from nitrogen, oxygen, and sulfur;
each Cy^{B2} is independently an optionally ring selected from phenyl, a 3-10 membered saturated or partially unsaturated monocyclic or bicyclic carbocyclic or heterocyclic ring having 1-4 heteroatoms independently selected from nitrogen, oxygen, and sulfur, and a 5-10 membered monocyclic or bicyclic aryl or heteroaryl with 1-4 heteroatoms independently selected from nitrogen, oxygen and sulfur;
each R is independently hydrogen, or an optionally substituted group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-10 membered heteroaryl ring having 1-4 heteroatoms independently selected from nitrogen, oxygen, and sulfur, or:
   two R groups on the same atom or adjacent atoms are optionally taken together with their intervening atoms to form a 3-10 membered saturated or partially unsaturated monocyclic, bicyclic, bridged bicyclic, or spirocyclic ring having 0-3 heteroatoms, in addition to the atom or adjacent atoms to which they are attached, independently selected from nitrogen, oxygen, and sulfur;
L^{x} is a covalent bond or an optionally substituted C₁₋₅ bivalent straight or branched saturated or unsaturated hydrocarbon chain wherein 1-2 methylene units of the chain are independently and optionally replaced with —O—, —C(O)—, —C(S)—, —CR₂—, —NR—, —C(NR)—, —S—, —S(O)—, —S(O)₂—, —S(O)(NR)—, or —CR═CR—; and
each of w, x, and y are independently 0, 1, 2, 3, or 4.

In some embodiments, the present invention provides a compound of formula I-a″″-1

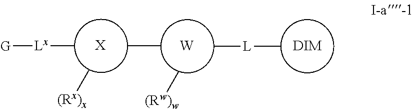

or a pharmaceutically acceptable salt thereof, wherein each L and DIM is as defined and described above and herein, wherein:
Ring W is a 9-membered bicyclic heteroaryl with 1-4 heteroatoms independently selected from nitrogen, oxygen, and sulfur;
Ring X is a 6-membered saturated or partially unsaturated carbocyclylenyl or heterocyclylenyl with 1-3 heteroatoms independently selected from nitrogen, oxygen, and sulfur;
G is hydrogen, halogen, or

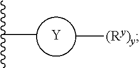

Ring Y is a 3- to 6-membered saturated or partially unsaturated carbocyclyl, or 4- to 6-membered monocyclic saturated or partially unsaturated heterocyclyl or heteroaryl ring with 1-4 heteroatoms independently selected from nitrogen, oxygen, or sulfur;
R^{w}, R^{x}, and R^{y} are independently selected from hydrogen, R^{A}, halogen, —CN, —NO₂, —OR, —SR, —NR₂, —SiR₃, —S(O)R, —S(O)₂R, —S(O)(NR)R, —S(O)₂NR₂, —S(O)R, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R;
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-10 membered monocyclic or bicyclic heteroaryl ring having 1-4 heteroatoms independently selected from nitrogen, oxygen, and sulfur;
L^{X} is a covalent bond or a C₁₋₅ bivalent straight or branched saturated or unsaturated hydrocarbon chain wherein 1-2 methylene units of the chain are independently and optionally replaced with —O—, —C(O)—, —C(S)—, —CR₂—, —NR—, —S—, —S(O)—, or —S(O)₂—;
each of w, x, and y are independently 0, 1, 2, 3, or 4; and
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same or adjacent atoms are taken together with their intervening atoms to form an optionally substituted 3-11 membered saturated or partially unsaturated monocyclic, bicyclic, bridged bicyclic, or spirocyclic carbocyclic or heterocyclic ring having 0-3 heteroatoms independently selected from nitrogen, oxygen, and sulfur.

As described above and defined herein, Ring W is 9-membered bicyclic heteroaryl with 1-4 heteroatoms independently selected from nitrogen, oxygen, and sulfur.

In some embodiments, Ring W is a 9-membered bicyclic heteroaryl with 1-2 heteroatoms independently selected from nitrogen, oxygen, and sulfur.

In some embodiments, Ring W is a 9-membered bicyclic heteroaryl with 1 heteroatom independently selected from nitrogen, oxygen, and sulfur. In some embodiments, Ring W is a 9-membered bicyclic heteroaryl with 2 heteroatoms independently selected from nitrogen, oxygen, and sulfur. In some embodiments, Ring W is indolyl, benzofuranyl, benzothiophenyl, benzimidazolyl, benzoxazolyl, thienopyridinyl, pyrrolo[3,2-b]pyridinyl, pyrrolo[2,3,-b]pyridinyl, pyrazolyl[1,5-a]pyridinyl, or imidazo[1,2-a]pyridinyl.

In some embodiments, Ring W is azaindazolyl (e.g., 4-, 5-, 6-, or 7-azaindazolyl). In some embodiments, Ring W is pyrrolol[2,3-c]pyridinyl. In some embodiments, Ring W is indolizinyl.

It will be understood that, in the preceding or following paragraphs where only a single point of attachment

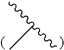

at Ring W is depicted, the depicted

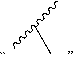

represents the point of attachment to Ring X, and an additional point of attachment to -L-DIM may replace an —H at any point on Ring W as allowed by valency.

In some embodiments, Ring W is

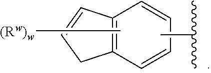

In some embodiments, Ring W is

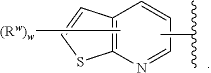

In some embodiments, Ring W is

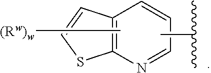

In some embodiments, Ring W is

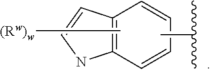

In some embodiments, Ring W is

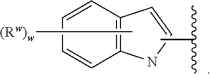

In some embodiments, Ring W is

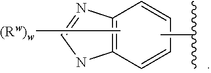

In some embodiments, Ring W is

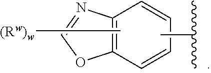

In some embodiments, Ring W is

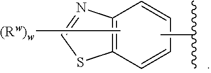

In some embodiments, Ring W is

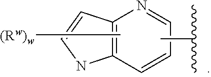

In some embodiments, Ring W is

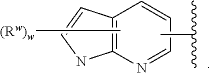

In some embodiments, Ring W is

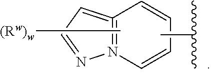

In some embodiments, Ring W is

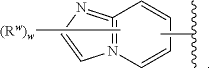

In some embodiments, Ring W is

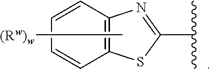

In some embodiments, Ring W is

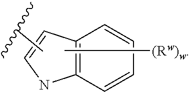

In some embodiments, Ring W is

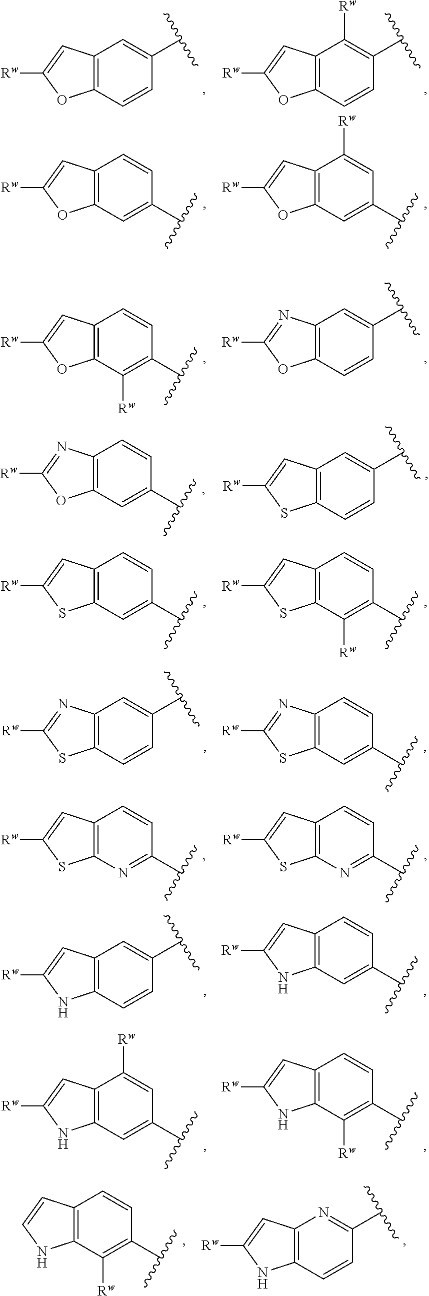

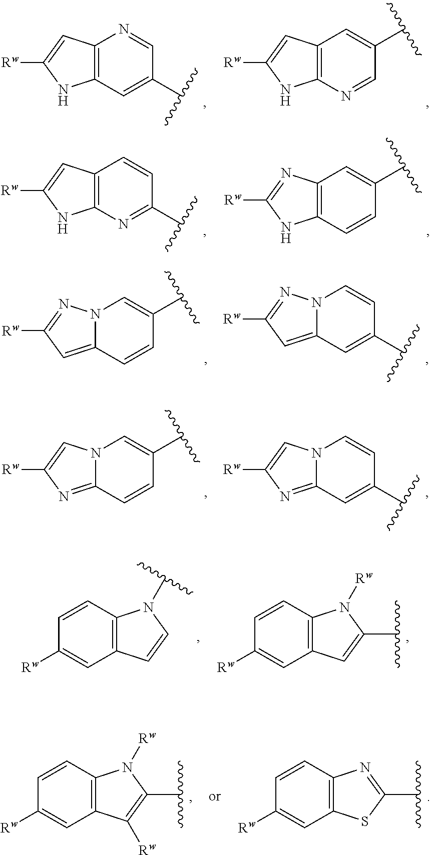

In some embodiment, Ring W is as depicted in the compounds of Table 1, below.

In some embodiments, Ring W is

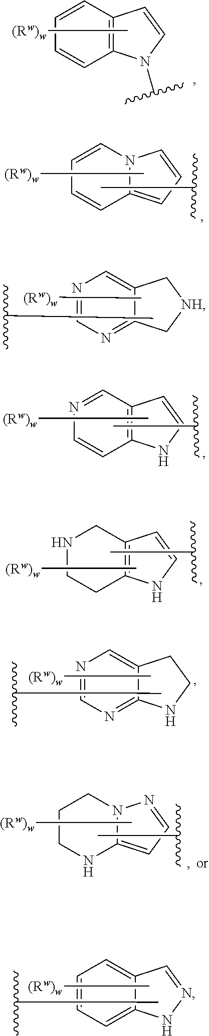

In some embodiments, Ring W is

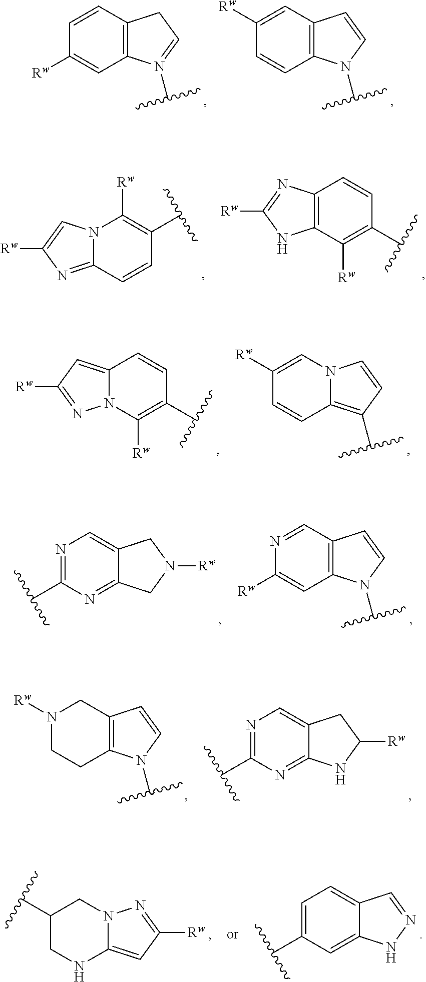

In some embodiments, Ring W is

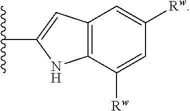

In some embodiments, Ring W is

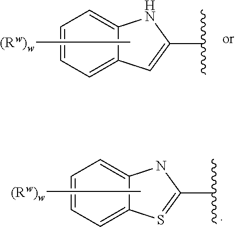

In some embodiments, Ring W is

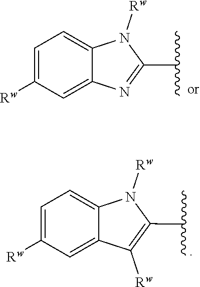

In some embodiments, Ring W is

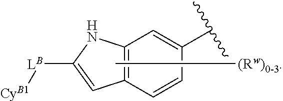

In some embodiments, Ring W is

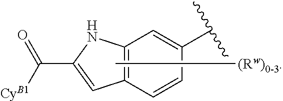

In some embodiments, Ring W and its R^{w} substituents are

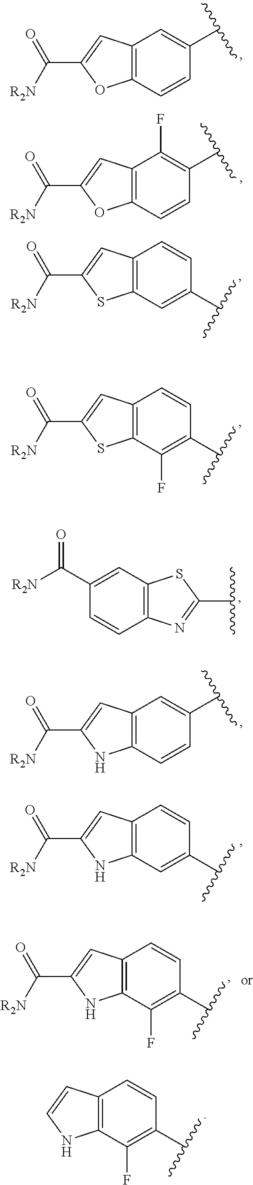

In some embodiments, Ring W and its R^{w} substituents are,

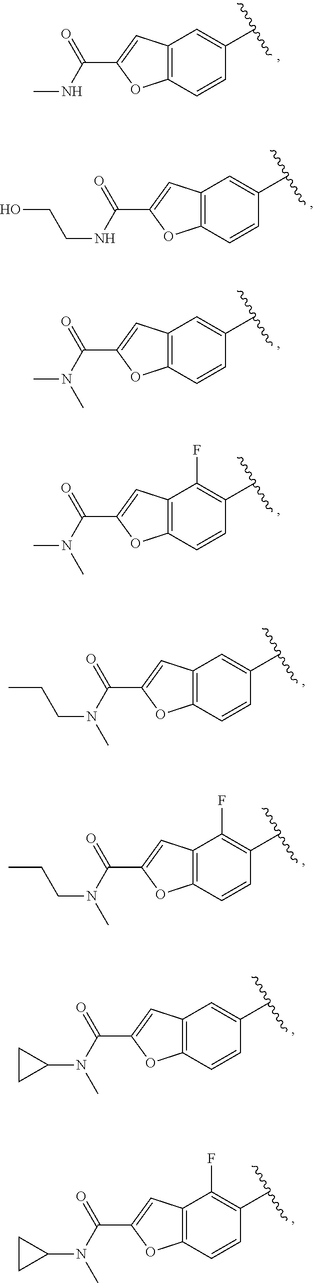

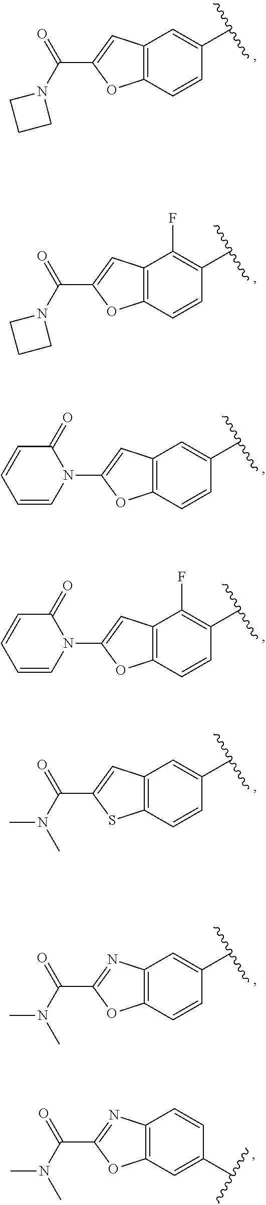

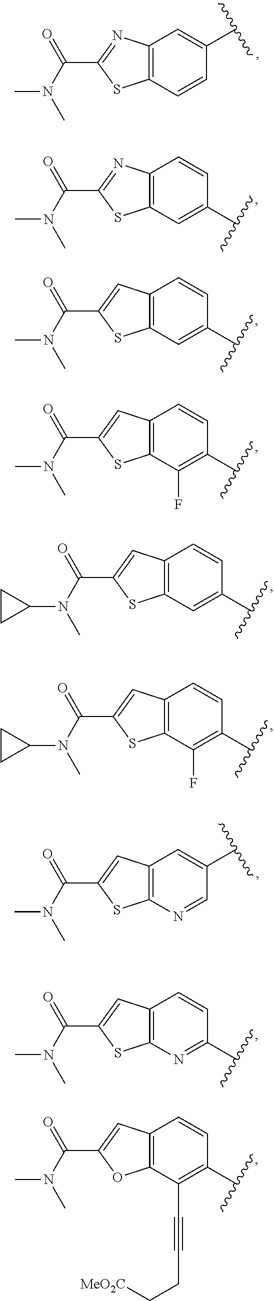

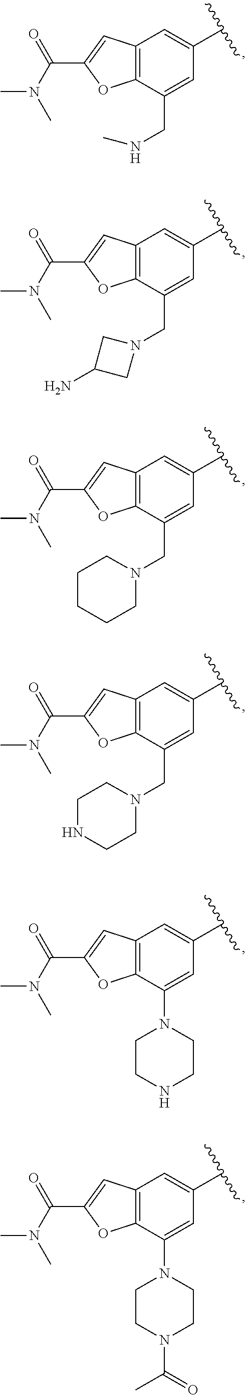 [SMILES: *c1cc(CNC)c2oc(C(=O)N(C)C)cc2c1 ; *c1cc(CN2CC(N)C2)c2oc(C(=O)N(C)C)cc2c1 ; *c1cc(CN2CCCCC2)c2oc(C(=O)N(C)C)cc2c1 ; *c1cc(CN2CCNCC2)c2oc(C(=O)N(C)C)cc2c1 ; *c1cc(N2CCNCC2)c2oc(C(=O)N(C)C)cc2c1 ; *c1cc(N2CCN(C(C)=O)CC2)c2oc(C(=O)N(C)C)cc2c1]

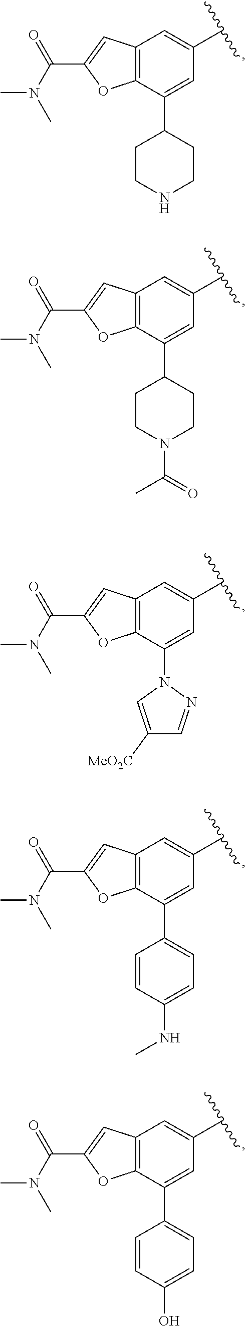 [SMILES: *c1cc(C2CCNCC2)c2oc(C(=O)N(C)C)cc2c1 ; *c1cc(C2CCN(C(C)=O)CC2)c2oc(C(=O)N(C)C)cc2c1 ; *c1cc(-n2cc(C(=O)OC)cn2)c2oc(C(=O)N(C)C)cc2c1 ; *c1cc(-c2ccc(NC)cc2)c2oc(C(=O)N(C)C)cc2c1 ; *c1cc(-c2ccc(O)cc2)c2oc(C(=O)N(C)C)cc2c1]

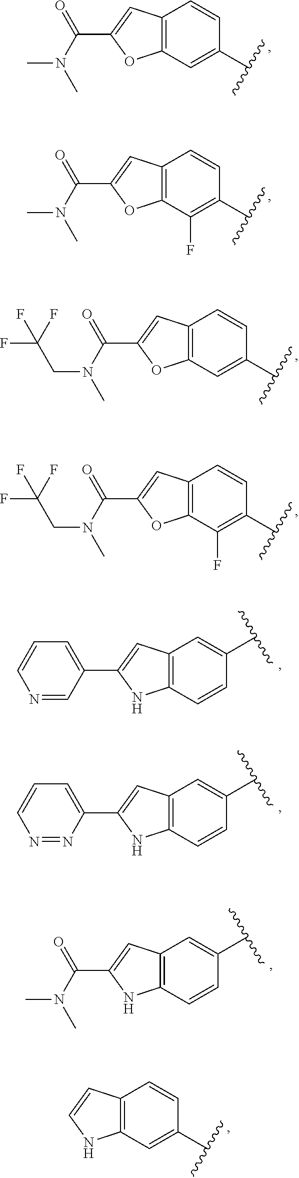

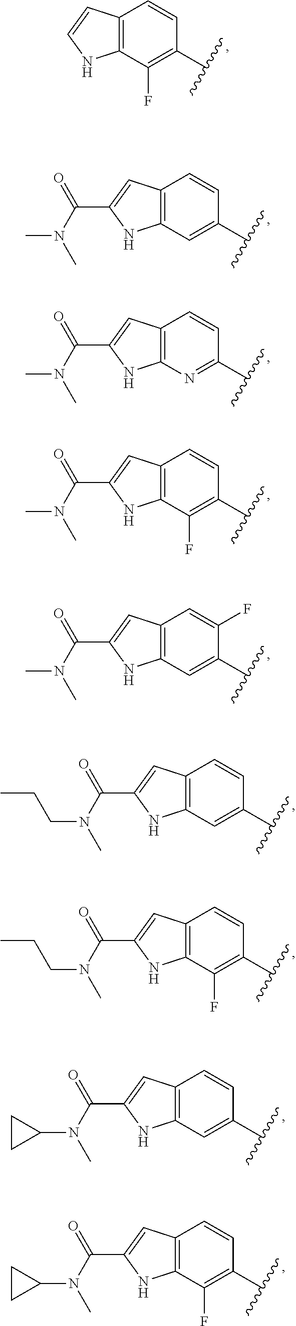

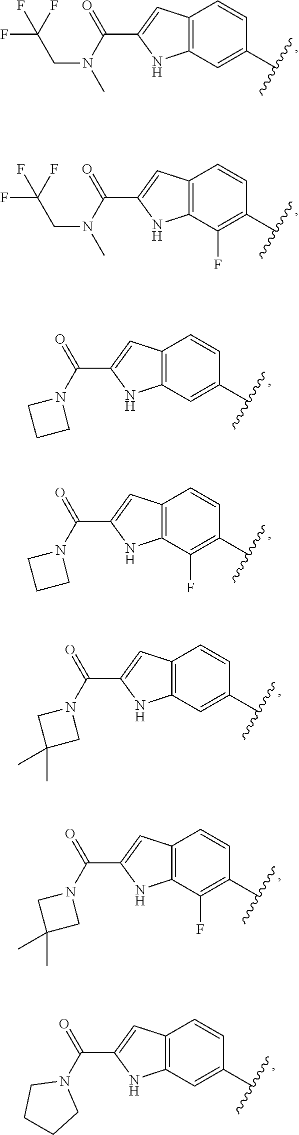

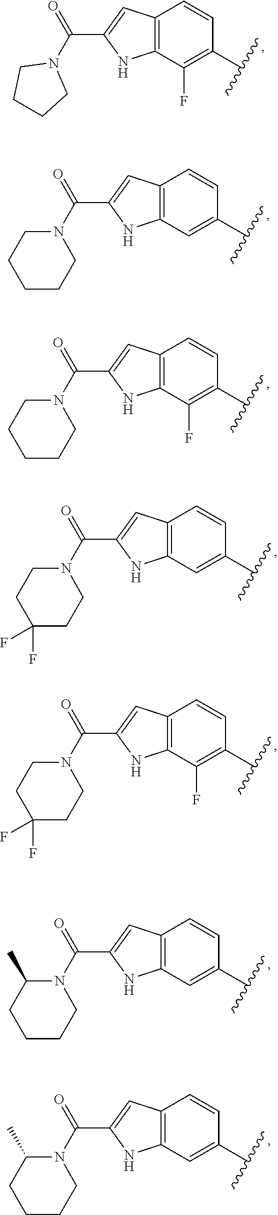

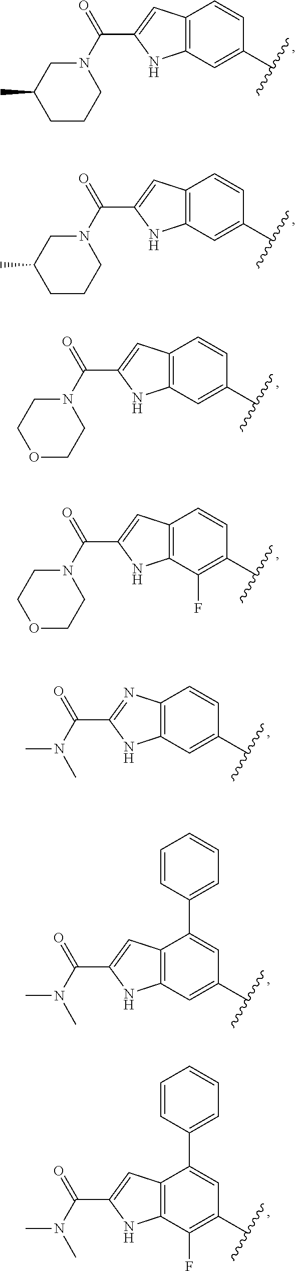

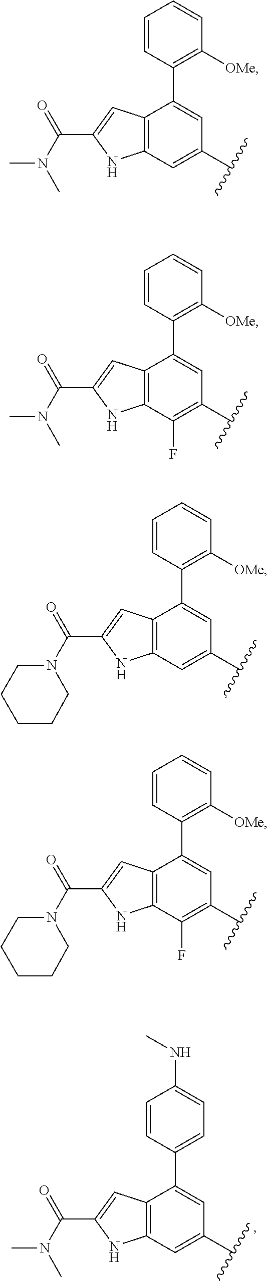 [SMILES: *c1cc(-c2ccccc2OC)c2cc(C(=O)N(C)C)[nH]c2c1 ; *c1cc(-c2ccccc2OC)c2cc(C(=O)N(C)C)[nH]c2c1F ; *c1cc(-c2ccccc2OC)c2cc(C(=O)N3CCCCC3)[nH]c2c1 ; *c1cc(-c2ccccc2OC)c2cc(C(=O)N3CCCCC3)[nH]c2c1F ; *c1cc(-c2ccc(NC)cc2)c2cc(C(=O)N(C)C)[nH]c2c1]

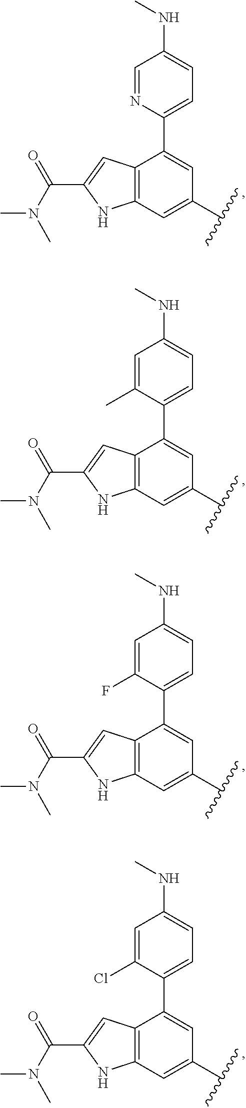 [SMILES: *c1cc(-c2ccc(NC)cn2)c2cc(C(=O)N(C)C)[nH]c2c1 ; *c1cc(-c2ccc(NC)cc2C)c2cc(C(=O)N(C)C)[nH]c2c1 ; *c1cc(-c2ccc(NC)cc2F)c2cc(C(=O)N(C)C)[nH]c2c1 ; *c1cc(-c2ccc(NC)cc2Cl)c2cc(C(=O)N(C)C)[nH]c2c1]

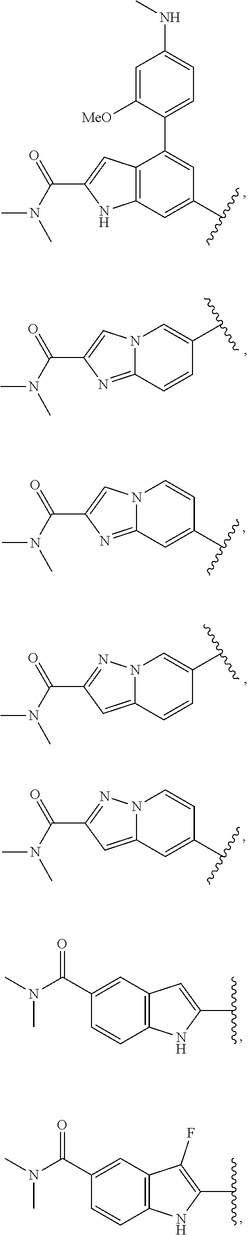

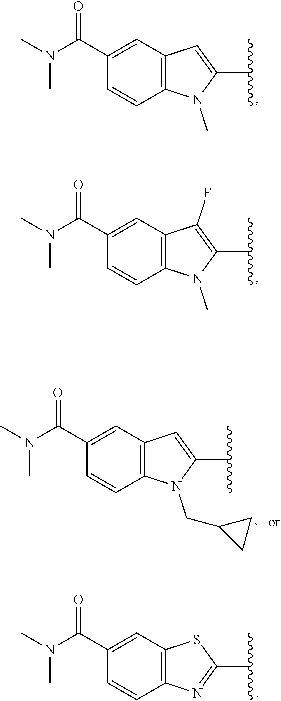 [SMILES: *c1cc2cc(C(=O)N(C)C)ccc2n1C ; *c1c(F)c2cc(C(=O)N(C)C)ccc2n1C ; *c1cc2cc(C(=O)N(C)C)ccc2n1CC1CC1 ; *c1nc2ccc(C(=O)N(C)C)cc2s1]

In some embodiments, Ring Wand its R^{W} substituents are

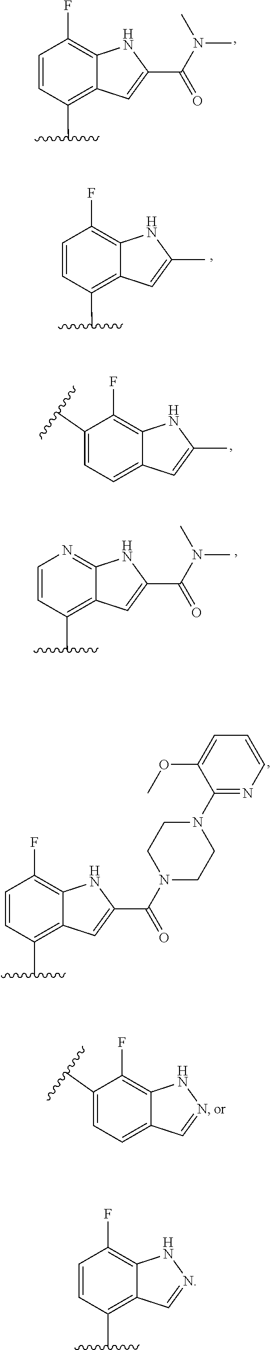

In some embodiments, Ring W and its R^{W} substituents are

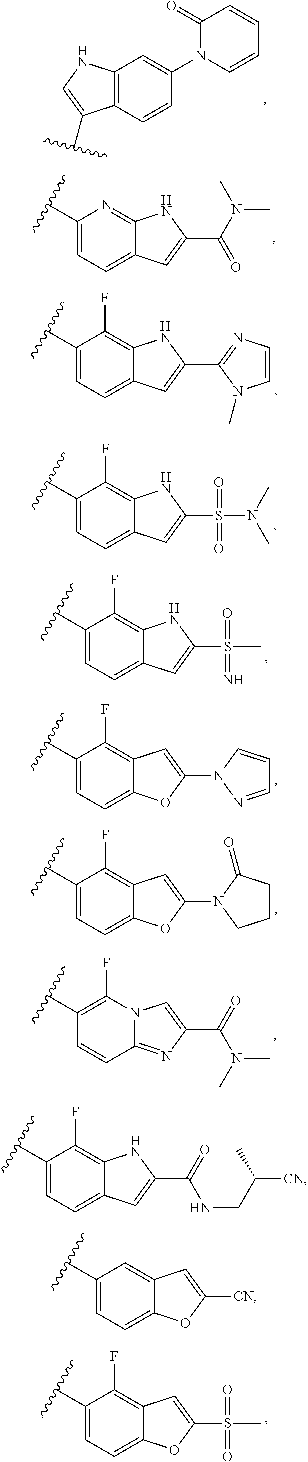

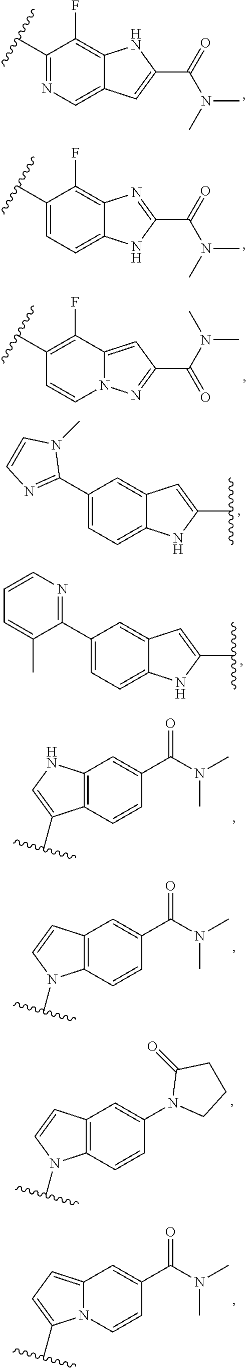

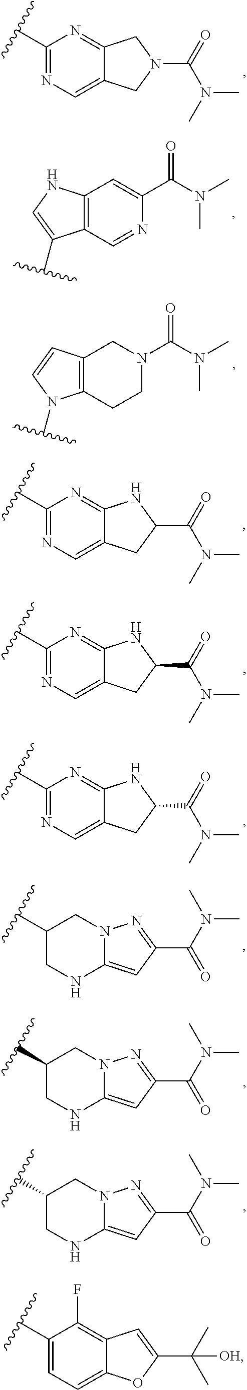

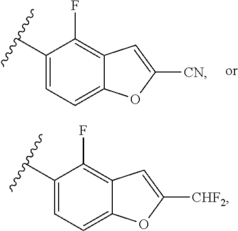

In some embodiments, Ring W and its R^{W} substituents are

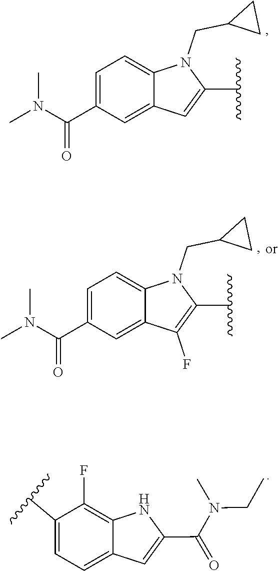 [SMILES: *c1cc2cc(C(=O)N(C)C)ccc2n1CC1CC1 ; *c1c(F)c2cc(C(=O)N(C)C)ccc2n1CC1CC1 ; *c1ccc2cc(C(=O)N(C)CC)[nH]c2c1F]

In some embodiments, Ring W and its R^{w} substituents are

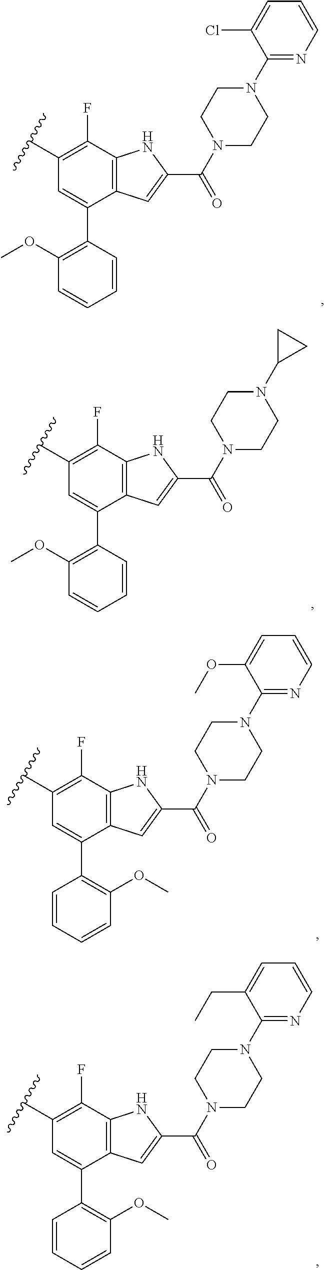 [SMILES: *c1cc(-c2ccccc2OC)c2cc(C(=O)N3CCN(c4ncccc4Cl)CC3)[nH]c2c1F ; *c1cc(-c2ccccc2OC)c2cc(C(=O)N3CCN(C4CC4)CC3)[nH]c2c1F ; *c1cc(-c2ccccc2OC)c2cc(C(=O)N3CCN(c4ncccc4OC)CC3)[nH]c2c1F ; *c1cc(-c2ccccc2OC)c2cc(C(=O)N3CCN(c4ncccc4CC)CC3)[nH]c2c1F]

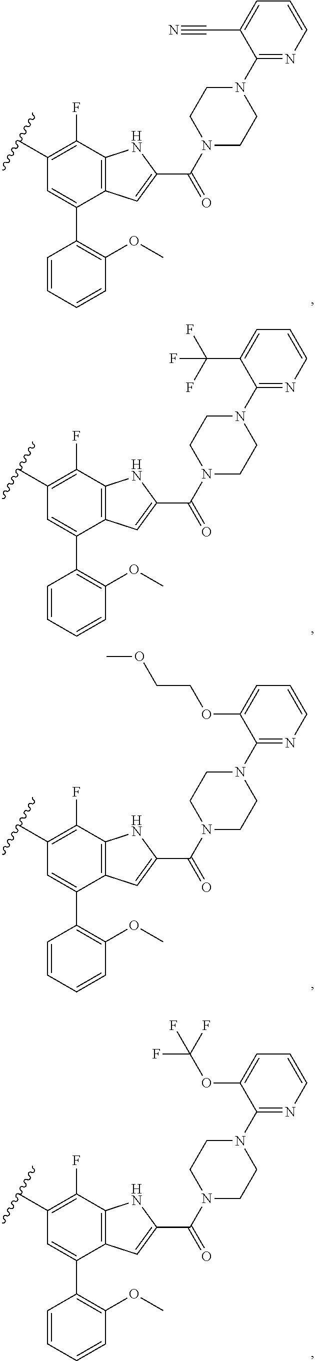

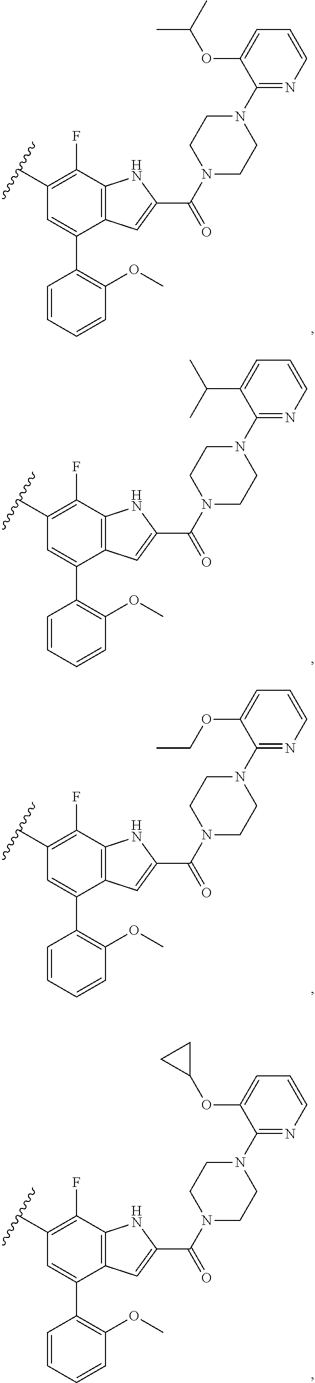 [SMILES: *c1cc(-c2ccccc2OC)c2cc(C(=O)N3CCN(c4ncccc4OC(C)C)CC3)[nH]c2c1F ; *c1cc(-c2ccccc2OC)c2cc(C(=O)N3CCN(c4ncccc4C(C)C)CC3)[nH]c2c1F ; *c1cc(-c2ccccc2OC)c2cc(C(=O)N3CCN(c4ncccc4OCC)CC3)[nH]c2c1F ; *c1cc(-c2ccccc2OC)c2cc(C(=O)N3CCN(c4ncccc4OC4CC4)CC3)[nH]c2c1F]

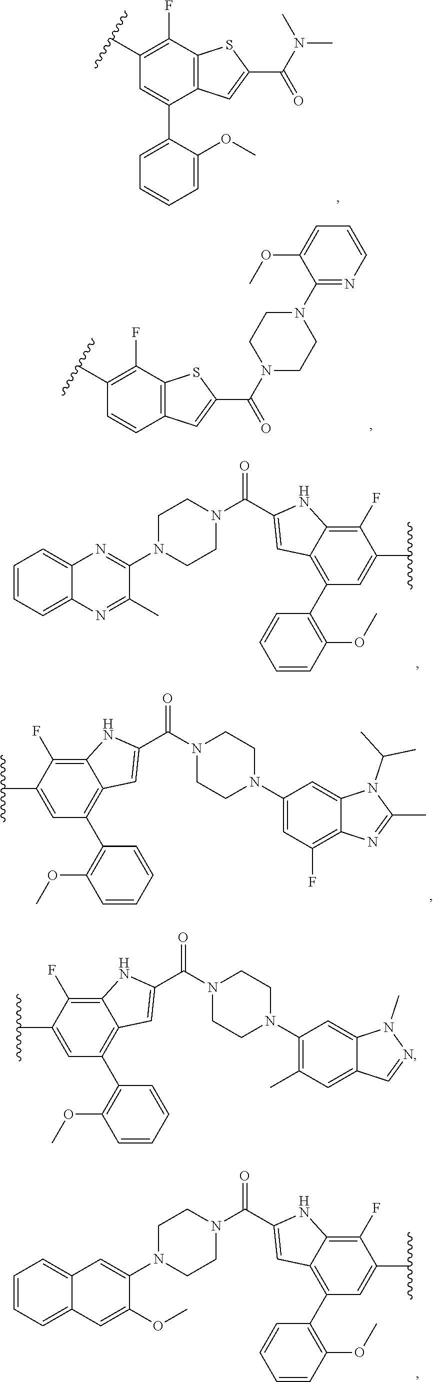

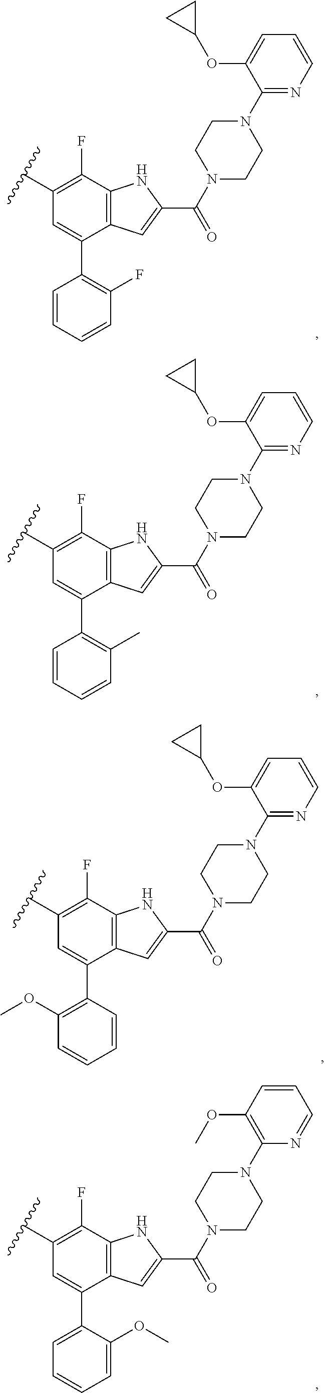 [SMILES: *c1cc(-c2ccccc2F)c2cc(C(=O)N3CCN(c4ncccc4OC4CC4)CC3)[nH]c2c1F ; *c1cc(-c2ccccc2C)c2cc(C(=O)N3CCN(c4ncccc4OC4CC4)CC3)[nH]c2c1F ; *c1cc(-c2ccccc2OC)c2cc(C(=O)N3CCN(c4ncccc4OC4CC4)CC3)[nH]c2c1F ; *c1cc(-c2ccccc2OC)c2cc(C(=O)N3CCN(c4ncccc4OC)CC3)[nH]c2c1F]

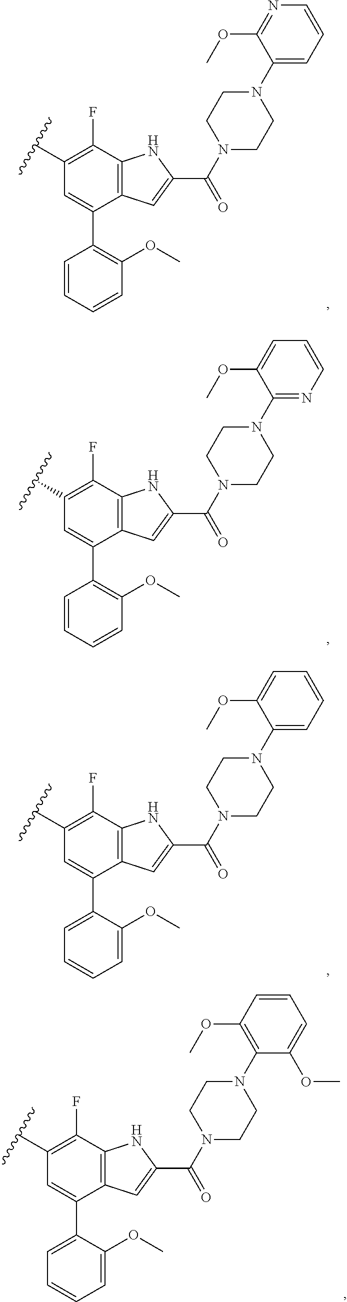

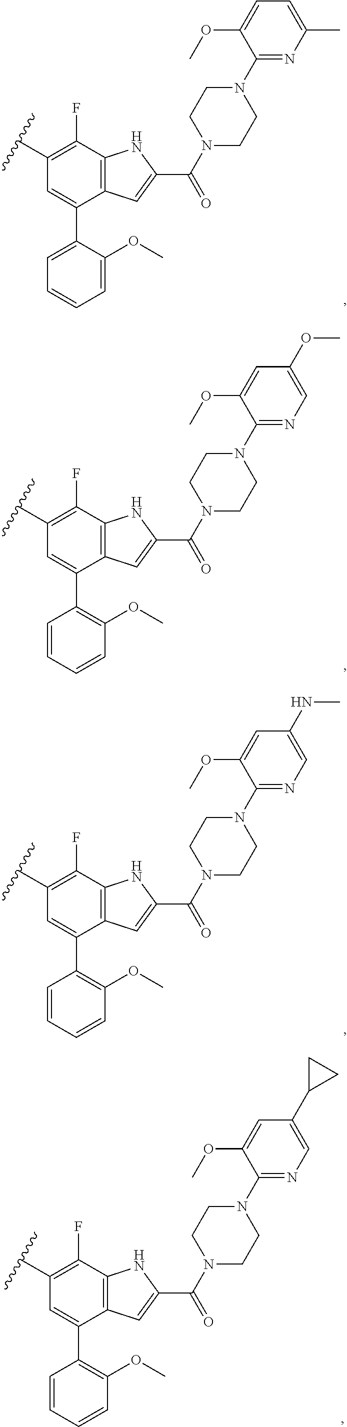 [SMILES: *c1cc(-c2ccccc2OC)c2cc(C(=O)N3CCN(c4nc(C)ccc4OC)CC3)[nH]c2c1F ; *c1cc(-c2ccccc2OC)c2cc(C(=O)N3CCN(c4ncc(OC)cc4OC)CC3)[nH]c2c1F ; *c1cc(-c2ccccc2OC)c2cc(C(=O)N3CCN(c4ncc(NC)cc4OC)CC3)[nH]c2c1F ; *c1cc(-c2ccccc2OC)c2cc(C(=O)N3CCN(c4ncc(C5CC5)cc4OC)CC3)[nH]c2c1F]

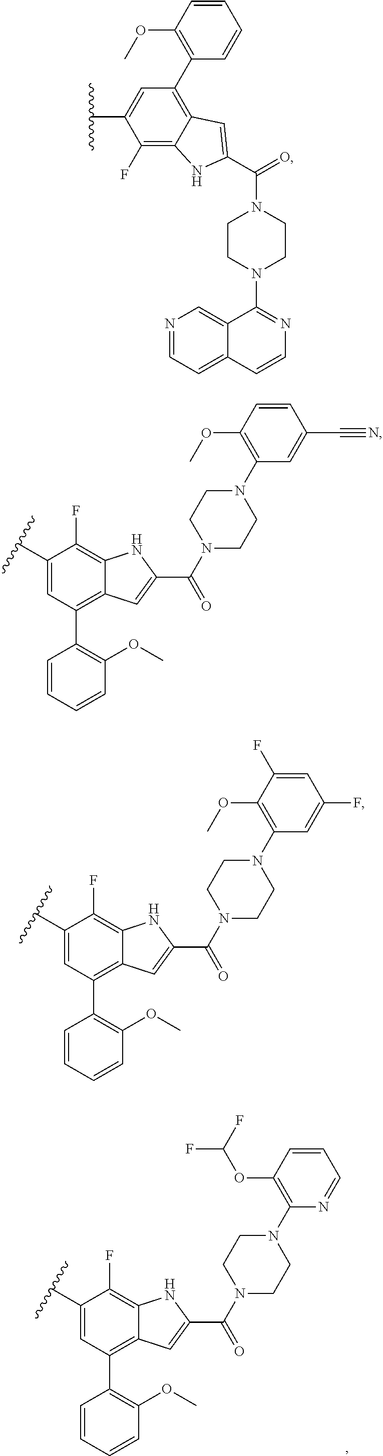

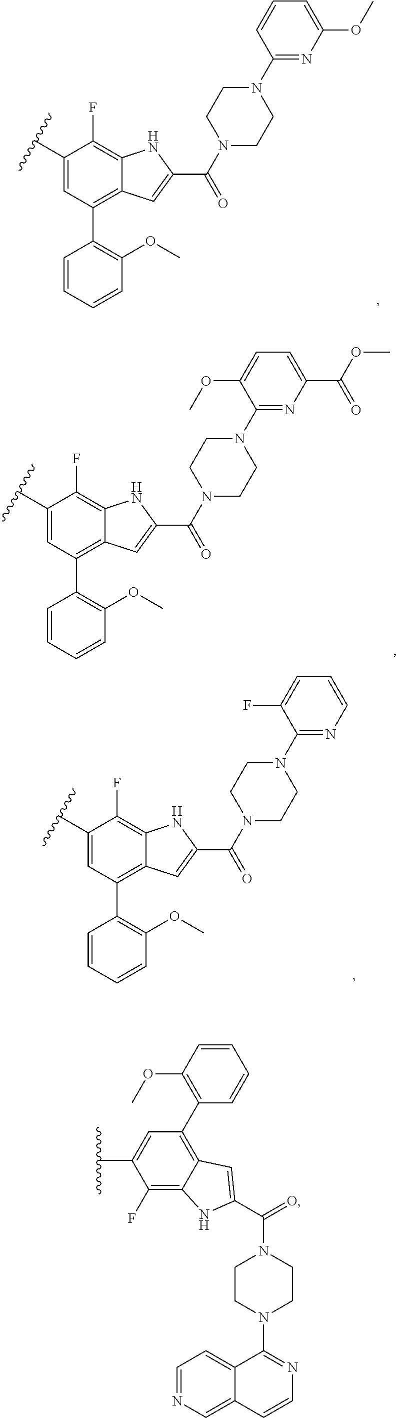 [SMILES: *c1cc(-c2ccccc2OC)c2cc(C(=O)N3CCN(c4cccc(OC)n4)CC3)[nH]c2c1F ; *c1cc(-c2ccccc2OC)c2cc(C(=O)N3CCN(c4nc(C(=O)OC)ccc4OC)CC3)[nH]c2c1F ; *c1cc(-c2ccccc2OC)c2cc(C(=O)N3CCN(c4ncccc4F)CC3)[nH]c2c1F ; *c1cc(-c2ccccc2OC)c2cc(C(=O)N3CCN(c4nccc5cnccc45)CC3)[nH]c2c1F]

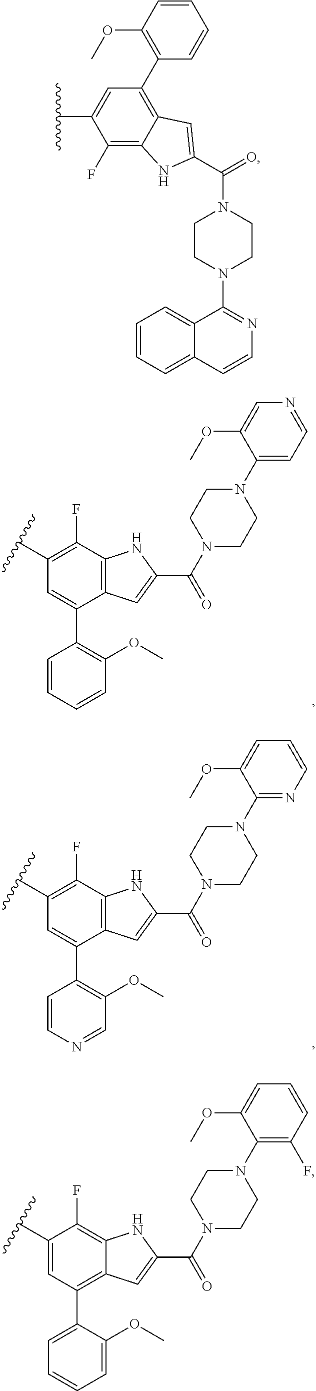

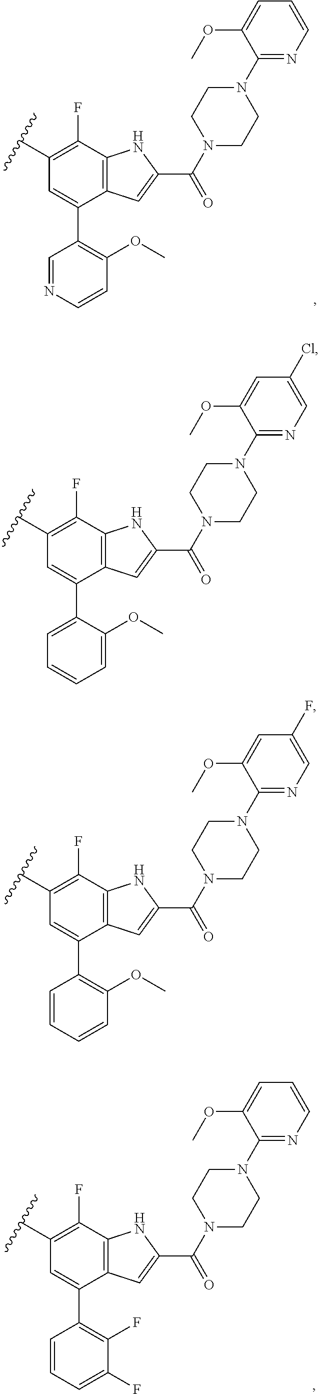 [SMILES: *c1cc(-c2cnccc2OC)c2cc(C(=O)N3CCN(c4ncccc4OC)CC3)[nH]c2c1F ; *c1cc(-c2ccccc2OC)c2cc(C(=O)N3CCN(c4ncc(Cl)cc4OC)CC3)[nH]c2c1F ; *c1cc(-c2ccccc2OC)c2cc(C(=O)N3CCN(c4ncc(F)cc4OC)CC3)[nH]c2c1F ; *c1cc(-c2cccc(F)c2F)c2cc(C(=O)N3CCN(c4ncccc4OC)CC3)[nH]c2c1F]

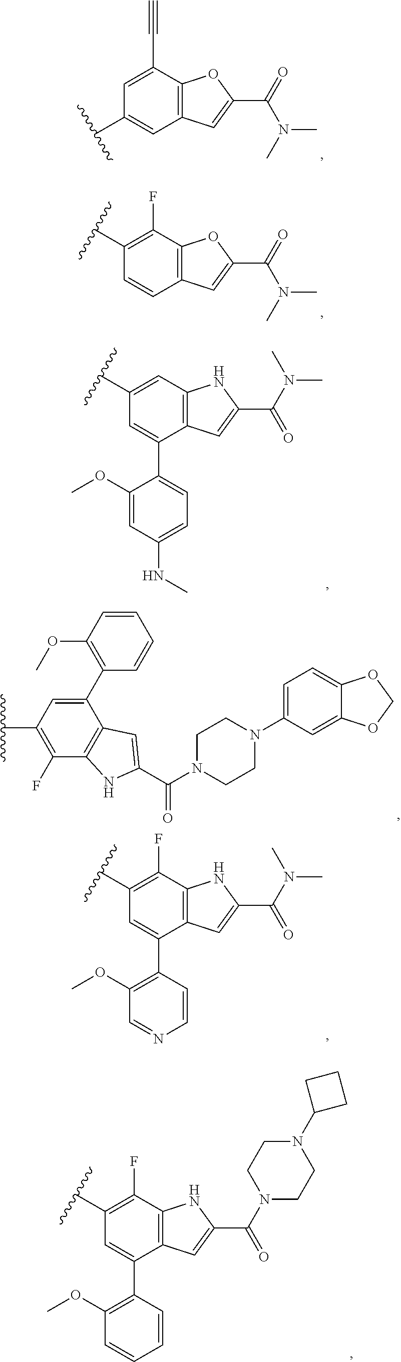

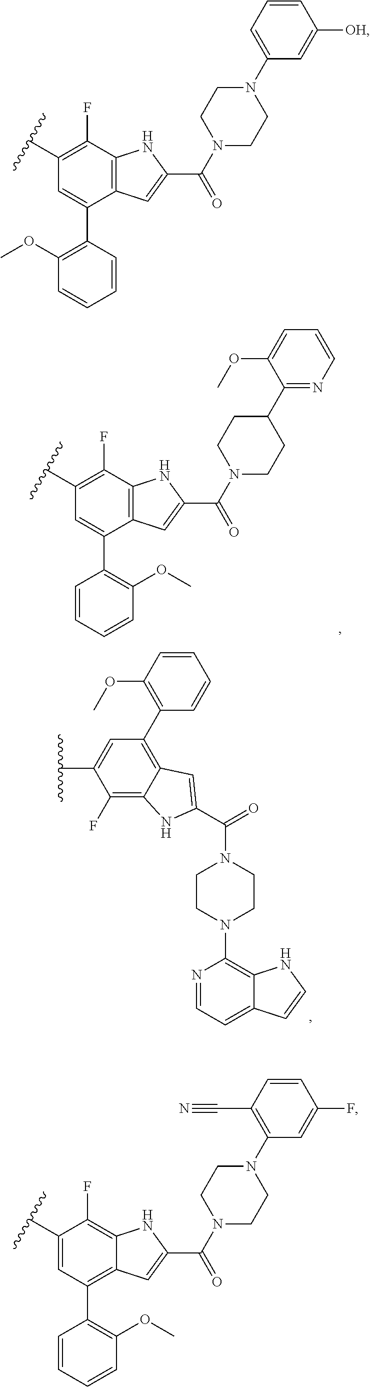

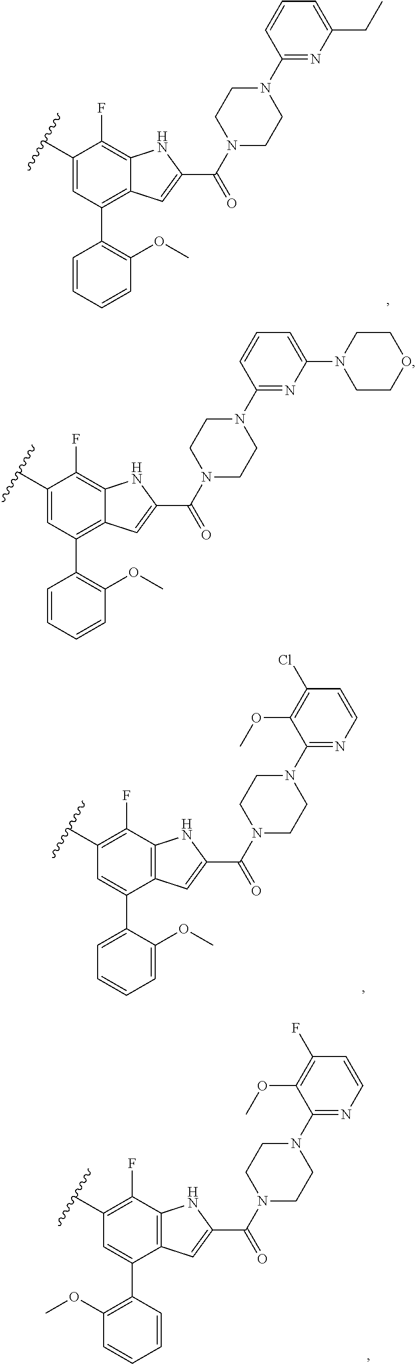

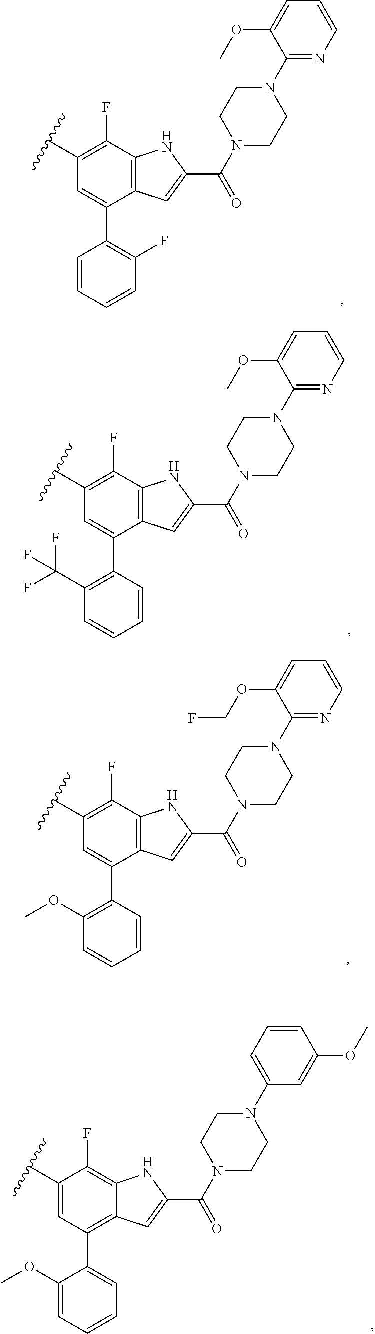

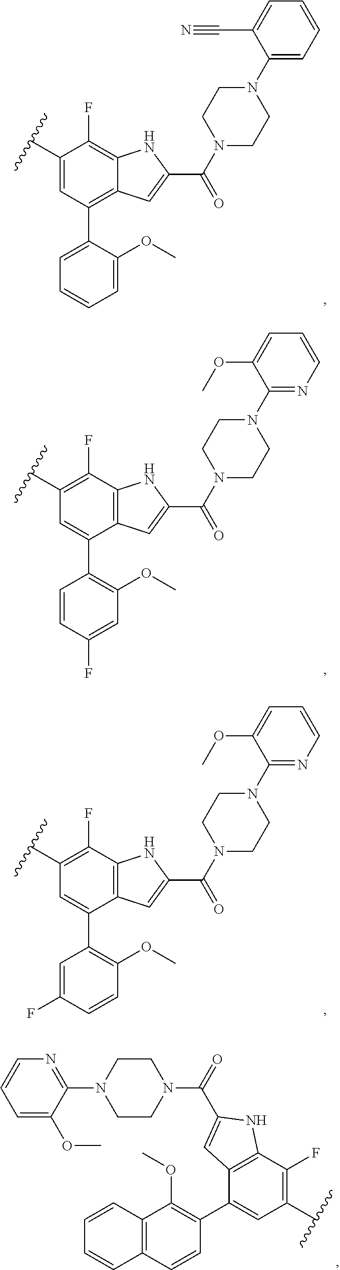 [SMILES: *c1cc(-c2ccccc2OC)c2cc(C(=O)N3CCN(c4ccccc4C#N)CC3)[nH]c2c1F ; *c1cc(-c2ccc(F)cc2OC)c2cc(C(=O)N3CCN(c4ncccc4OC)CC3)[nH]c2c1F ; *c1cc(-c2cc(F)ccc2OC)c2cc(C(=O)N3CCN(c4ncccc4OC)CC3)[nH]c2c1F ; *c1cc(-c2ccc3ccccc3c2OC)c2cc(C(=O)N3CCN(c4ncccc4OC)CC3)[nH]c2c1F]

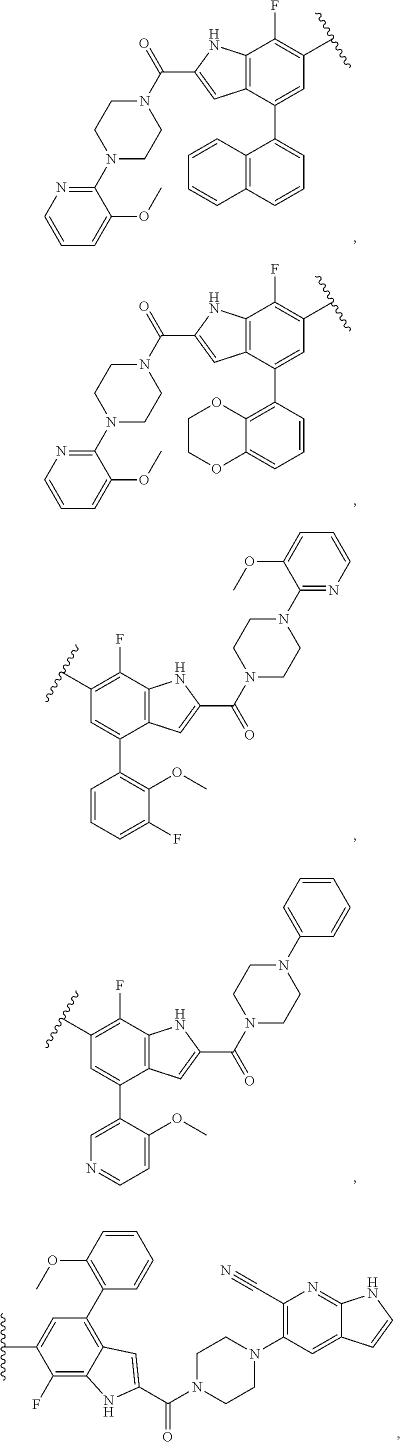 [SMILES: *c1cc(-c2cccc3ccccc23)c2cc(C(=O)N3CCN(c4ncccc4OC)CC3)[nH]c2c1F ; *c1cc(-c2cccc3c2OCCO3)c2cc(C(=O)N3CCN(c4ncccc4OC)CC3)[nH]c2c1F ; *c1cc(-c2cccc(F)c2OC)c2cc(C(=O)N3CCN(c4ncccc4OC)CC3)[nH]c2c1F ; *c1cc(-c2cnccc2OC)c2cc(C(=O)N3CCN(c4ccccc4)CC3)[nH]c2c1F ; *c1cc(-c2ccccc2OC)c2cc(C(=O)N3CCN(c4cc5cc[nH]c5nc4C#N)CC3)[nH]c2c1F]

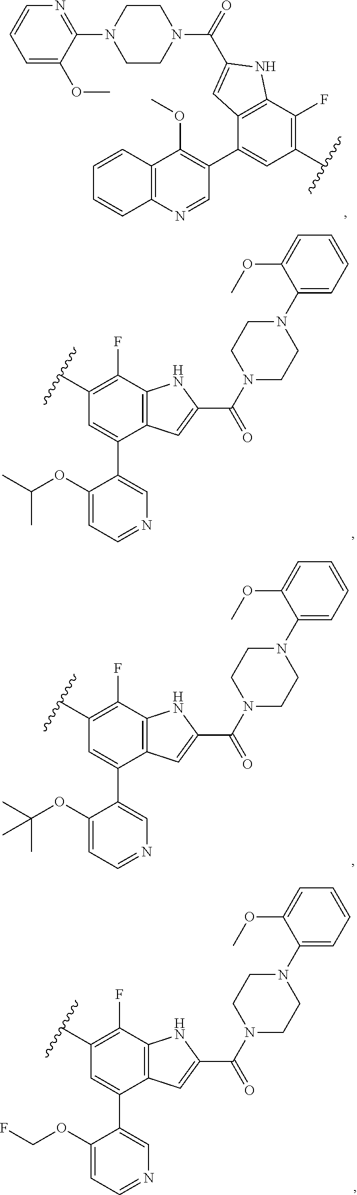 [SMILES: *c1cc(-c2cnc3ccccc3c2OC)c2cc(C(=O)N3CCN(c4ncccc4OC)CC3)[nH]c2c1F ; *c1cc(-c2cnccc2OC(C)C)c2cc(C(=O)N3CCN(c4ccccc4OC)CC3)[nH]c2c1F ; *c1cc(-c2cnccc2OC(C)(C)C)c2cc(C(=O)N3CCN(c4ccccc4OC)CC3)[nH]c2c1F ; *c1cc(-c2cnccc2OCF)c2cc(C(=O)N3CCN(c4ccccc4OC)CC3)[nH]c2c1F]

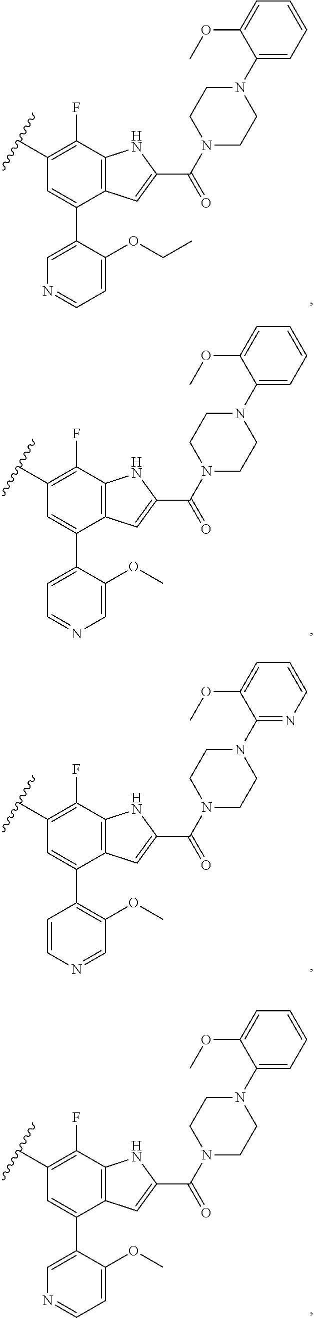 [SMILES: *c1cc(-c2cnccc2OCC)c2cc(C(=O)N3CCN(c4ccccc4OC)CC3)[nH]c2c1F ; *c1cc(-c2ccncc2OC)c2cc(C(=O)N3CCN(c4ccccc4OC)CC3)[nH]c2c1F ; *c1cc(-c2ccncc2OC)c2cc(C(=O)N3CCN(c4ncccc4OC)CC3)[nH]c2c1F ; *c1cc(-c2cnccc2OC)c2cc(C(=O)N3CCN(c4ccccc4OC)CC3)[nH]c2c1F]

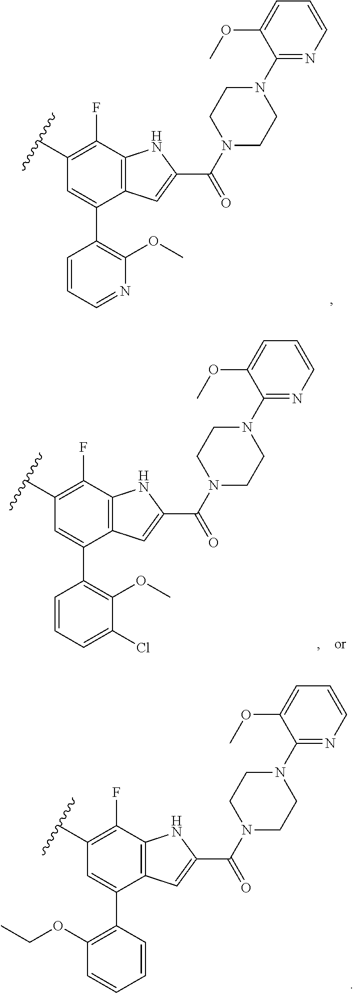 [SMILES: *c1cc(-c2cccnc2OC)c2cc(C(=O)N3CCN(c4ncccc4OC)CC3)[nH]c2c1F ; *c1cc(-c2cccc(Cl)c2OC)c2cc(C(=O)N3CCN(c4ncccc4OC)CC3)[nH]c2c1F ; *c1cc(-c2ccccc2OCC)c2cc(C(=O)N3CCN(c4ncccc4OC)CC3)[nH]c2c1F]

In some embodiments, Ring W is:

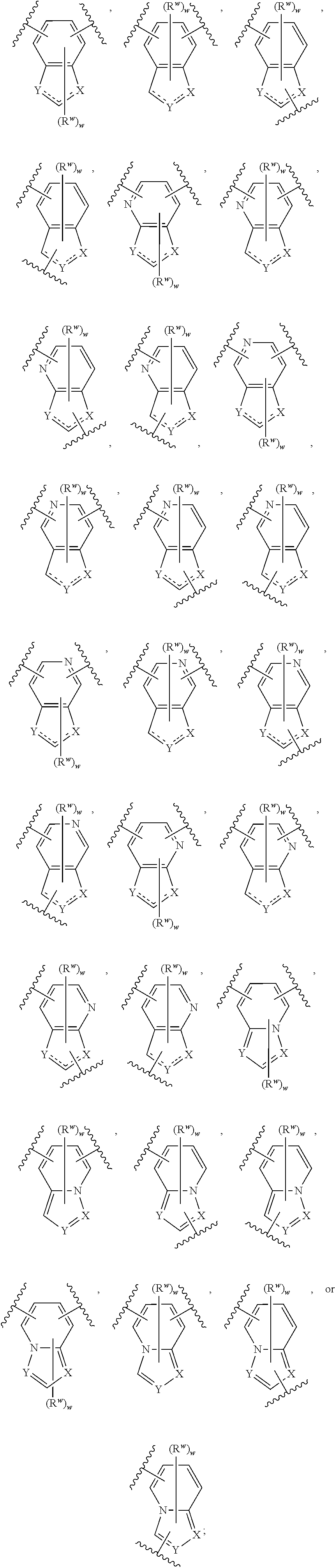

wherein R^{w} and w are as defined above and described herein; and
each of X and Y of Ring W is independently N, NH, N—R^{W}, —O—, —S—, C—H, C—R^{W}, C—H₂, CH(R^{W}), or C—(R^{W})₂.

In some embodiments, Ring W is:

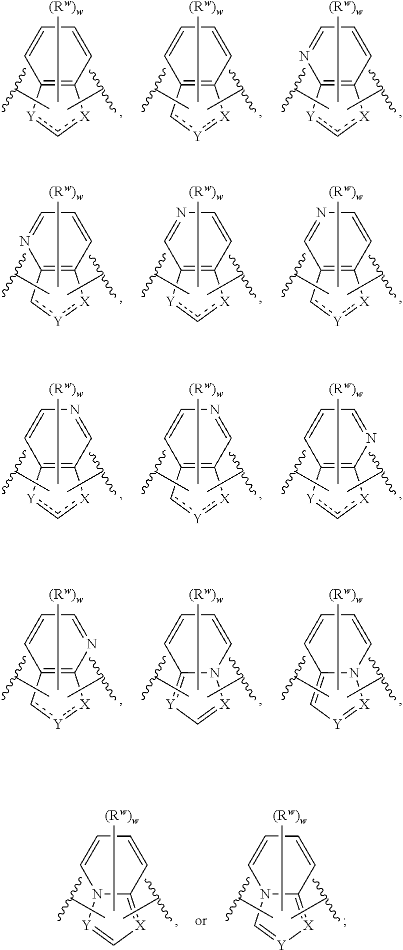

wherein each of R^{w}, w, X, and Y is as defined above and described here.

In some embodiments, Ring W is

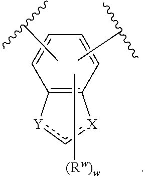

In some embodiments, Ring W is

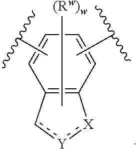

In some embodiments, Ring W is

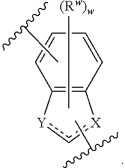

In some embodiments, Ring W is

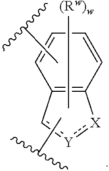

In some embodiments, Ring W is

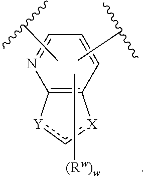

In some embodiments, Ring W is

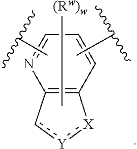

In some embodiments, Ring W is

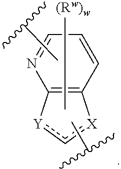

In some embodiments, Ring W is

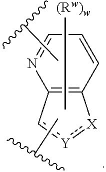

In some embodiments, Ring W is

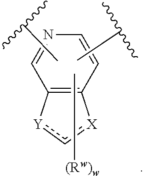

In some embodiments, Ring W is

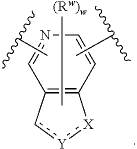

In some embodiments, Ring W is

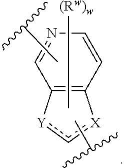

In some embodiments, Ring W is

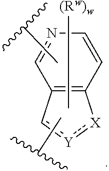

In some embodiments, Ring W is

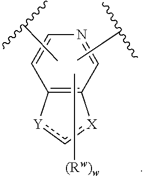

In some embodiments, Ring W is

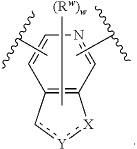

In some embodiments, Ring W is

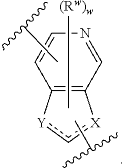

In some embodiments, Ring W is

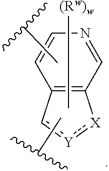

In some embodiments, Ring W is

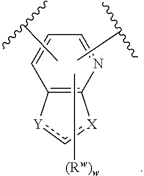

In some embodiments, Ring W is

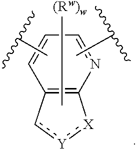

In some embodiments, Ring W is

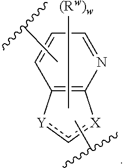

In some embodiments, Ring W is

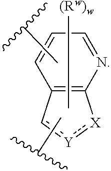

In some embodiments, Ring W is

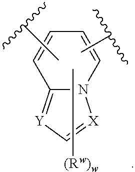

In some embodiments, Ring W is

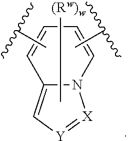

In some embodiments, Ring W is

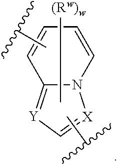

In some embodiments, Ring W is

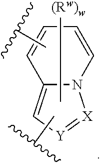

In some embodiments, Ring W is

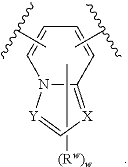

In some embodiments, Ring W is

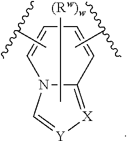

In some embodiments, Ring W is

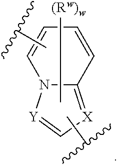

In some embodiments, Ring W is

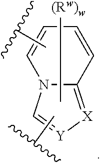

In some embodiments, each of X and Y of Ring W is independently N, NH, N—R^{W}, —O—, —S—, C—H, C—R^{W}, CH₂, CH(R^{W}), or C—(R^{W})₂.

In some embodiments, Ring W is

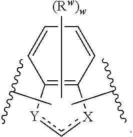

In some embodiments, Ring W is

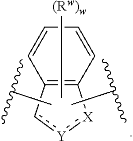

In some embodiments, Ring W is

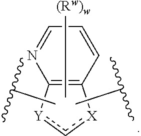

In some embodiments, Ring W is

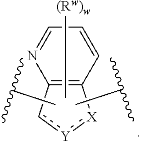

In some embodiments, Ring W is

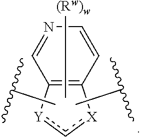

In some embodiments, Ring W is

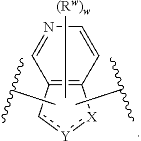

In some embodiments, Ring W is

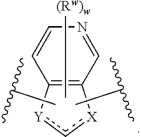

In some embodiments, Ring W is

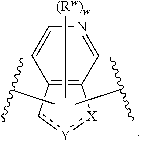

In some embodiments, Ring W is

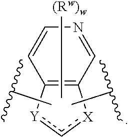

In some embodiments, Ring W is

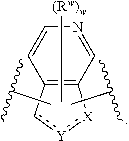

In some embodiments, Ring W is

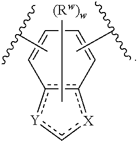

In some embodiments, Ring W is

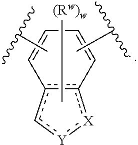

As defined above and described herein, each of X and Y of Ring W is independently N, NH, N—R^{W}, —O—, —S—, C—H, C—R^{W}, C—H₂, CH(R^{W}), or C—(R^{W})₂. In some embodiments, each of X and Y of Ring W is independently N, NH, N—R^{W}, —O—, —S—, C—H, C—R^{W}, C—H₂, CH(R^{W}), or C—(R^{W})₂, as allowed by valency.

In some embodiments, X is C—R^{w} or CH, and Y is N—R. In some embodiments, X is C—R^{w} or CH, and Y is S. In some embodiments, X is C—R^{w} or CH, and Y is O. In some embodiments, X is N—R^{w} or NH, and Y is C—R^{w} or CH. In some embodiments, X is S, and Y is C—R^{w} or CH. In some embodiments, X is O, and Y is C—R^{w} or CH.

In some embodiments, Ring W is

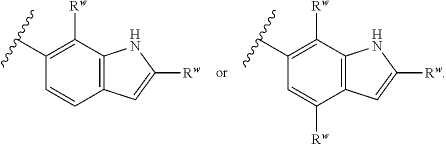

In some embodiments Ring W is

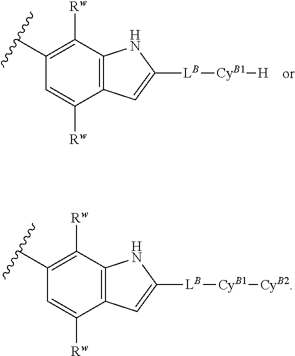

In some embodiments Ring W is

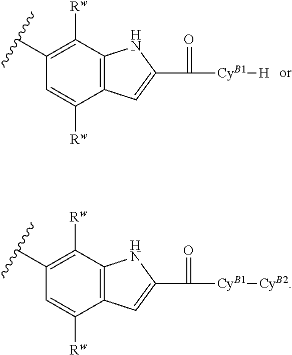

In some embodiments, Ring W is

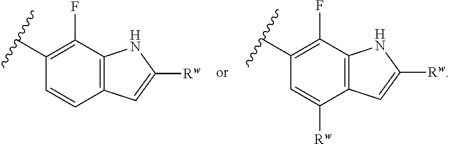

In some embodiments Ring W is

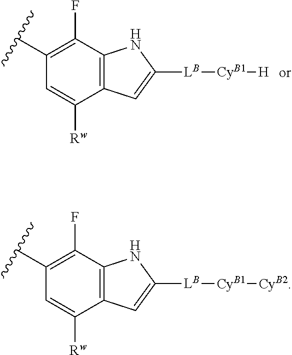

In some embodiments Ring W is

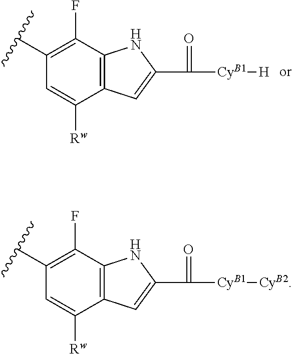

In some embodiments, Ring W is

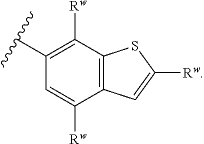

In some embodiments, Ring W is

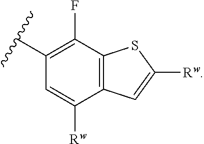

In some embodiments, Ring W is

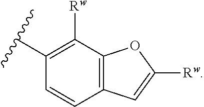

As described above and defined herein, Ring X is a 6-membered saturated or partially unsaturated carbocyclylenyl or heterocyclylenyl with 1-3 heteroatoms independently selected from nitrogen, oxygen, and sulfur.

In some embodiments, Ring X is a 6-membered saturated or partially unsaturated heterocyclylenyl with 1-3 heteroatoms independently selected from nitrogen, oxygen, and sulfur. In some embodiments, Ring X is a 6-membered saturated or partially unsaturated heterocyclylenyl having 1 nitrogen atom.

In some embodiments, Ring X is

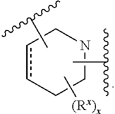

In some embodiments, Ring X is

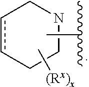

In some embodiments, Ring X is

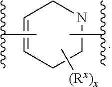

In some embodiments, Ring X is

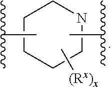

In some embodiments, Ring X is

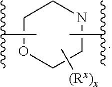

In some embodiments, Ring X is

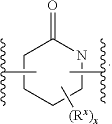

In some embodiments, Ring X is

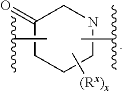

In some embodiments, Ring X is

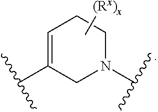

In some embodiments, Ring X is

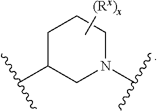

In some embodiments, Ring X is

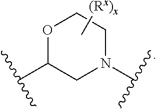

In some embodiments, Ring X is

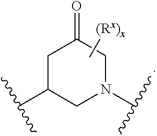

In some embodiments, Ring is

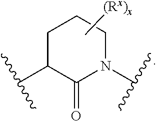

In some embodiments, Ring X is

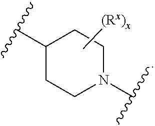

In some embodiments, Ring X and its R^{X} substituents are

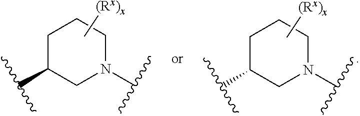

In some embodiments, Ring X is

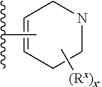

In some embodiments, Ring X is

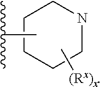

In some embodiments, Ring X is

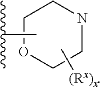

In some embodiments, Ring X is

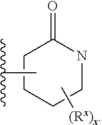

In some embodiments, Ring X is

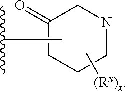

In some embodiments, Ring X is

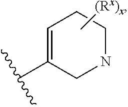

In some embodiments, Ring X is

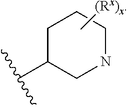

In some embodiments, Ring X is

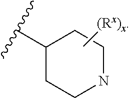

In some embodiments, Ring X is

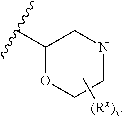

In some embodiments, Ring X is

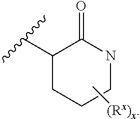

In some embodiments, Ring X is

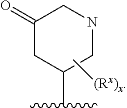

In some embodiment, Ring X is as depicted in the compounds of Table 1, below.

In some embodiments, Ring X and its R^{X} substituents are

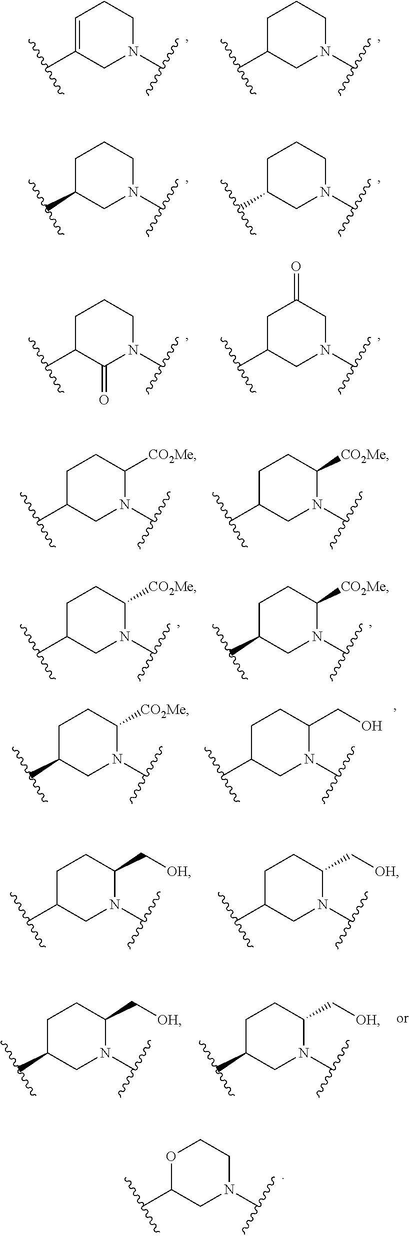

In some embodiments, Ring X and its R^{X} substituents are

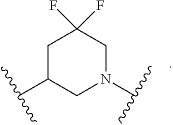 [SMILES: *C1CN(*)CC(F)(F)C1]

In some embodiments, Ring X and its R^{X} substituents are

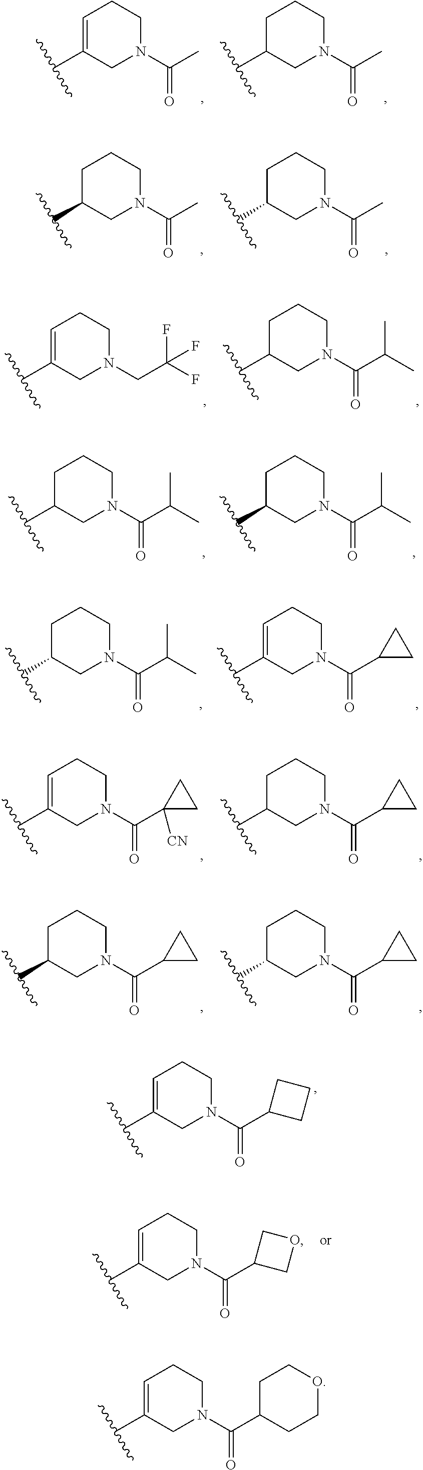

As described above and defined herein, G is hydrogen or

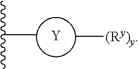

In some embodiments, G is hydrogen. In some embodiments, G is

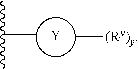

In some embodiment, G is as depicted in the compounds of Table 1, below.

As described above and defined herein, Ring Y is a 3- to 6-membered saturated or partially unsaturated carbocyclyl, or 4- to 6-membered monocyclic saturated or partially unsaturated heterocyclyl or heteroaryl ring with 1-4 heteroatoms independently selected from nitrogen, oxygen, or sulfur.

In some embodiments, Ring Y is a 5-6 membered saturated or partially unsaturated monocyclic carbocyclyl. In some embodiments, Ring Y is a 3 membered saturated or partially unsaturated monocyclic carbocyclyl. In some embodiments, Ring Y is a 4 membered saturated or partially unsaturated monocyclic carbocyclyl. In some embodiments, Ring Y is a 5 membered saturated or partially unsaturated monocyclic carbocyclyl. In some embodiments, Ring Y is a 6 membered saturated or partially unsaturated monocyclic carbocyclyl. In some embodiments, Ring Y is cyclopropyl, cyclobutyl, cyclopentyl, or cyclohexyl.

In some embodiments, Ring Y is a 3-6 membered saturated or partially unsaturated monocyclic carbocyclyl. In some embodiments, Ring Y is cyclopropyl. In some embodiments, Ring Y is cyclobutyl. In some embodiments, Ring Y is cyclopentyl. In some embodiments, Ring Y is hexyl.

In some embodiments, Ring Y is a 5-6 membered monocyclic heteroaryl with 1-4 heteroatoms independently selected from nitrogen, oxygen, and sulfur. In some embodiments, Ring Y is a 5-membered monocyclic heteroaryl with 1-4 heteroatoms independently selected from nitrogen, oxygen, and sulfur.

In some embodiments, Ring Y is a 5-membered monocyclic heteroaryl ring with 1 heteroatom selected from nitrogen, oxygen, and sulfur. In some embodiments, Ring Y is a 5-membered monocyclic heteroaryl ring with 2 heteroatoms independently selected from nitrogen, oxygen, and sulfur. In some embodiments, Ring Y is a 5-membered monocyclic heteroaryl ring with 3 heteroatoms independently selected from nitrogen, oxygen, and sulfur. In some embodiments, Ring Y is a 5-membered monocyclic heteroaryl ring with 4 heteroatoms independently selected from nitrogen, oxygen, and sulfur. In some embodiments, Ring Y is a 5-membered monocyclic heteroaryl ring with 2-3 nitrogen heteroatoms.

In some embodiments, Ring Y is a 6-membered monocyclic heteroaryl with 1-4 heteroatoms independently selected from nitrogen, oxygen, and sulfur. In some embodiments, Ring Y is a 6-membered monocyclic heteroaryl with 1-4 nitrogen heteroatoms. In some embodiments, Ring Y is pyridinyl, pyrimidinyl, pyridazinyl, or triazinyl.

In some embodiments, Ring Y is

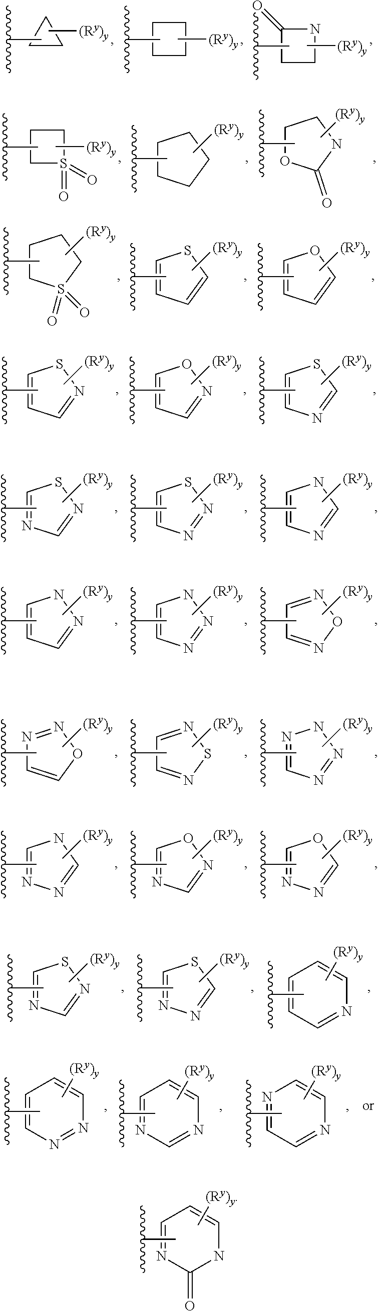

In some embodiments, Ring Y is

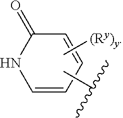

In some embodiments, Ring Y is

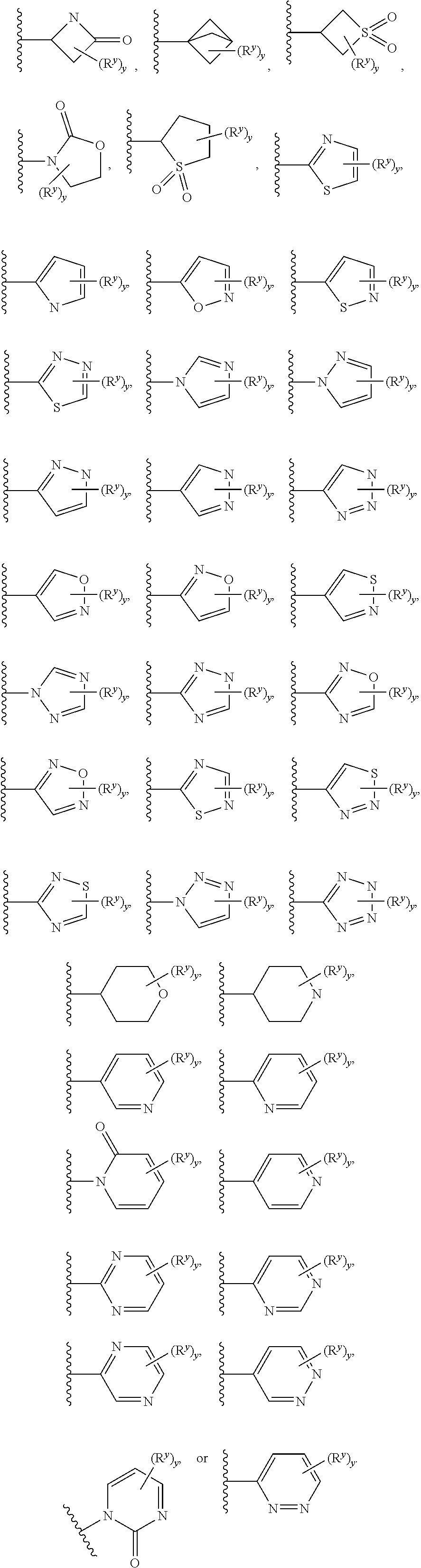

In some embodiment. Ring Y is

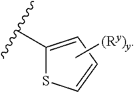

In some embodiment, Ring Y is as depicted in the compounds of Table 1, below.

In some embodiments, Ring Y and its R^{y} substituents are

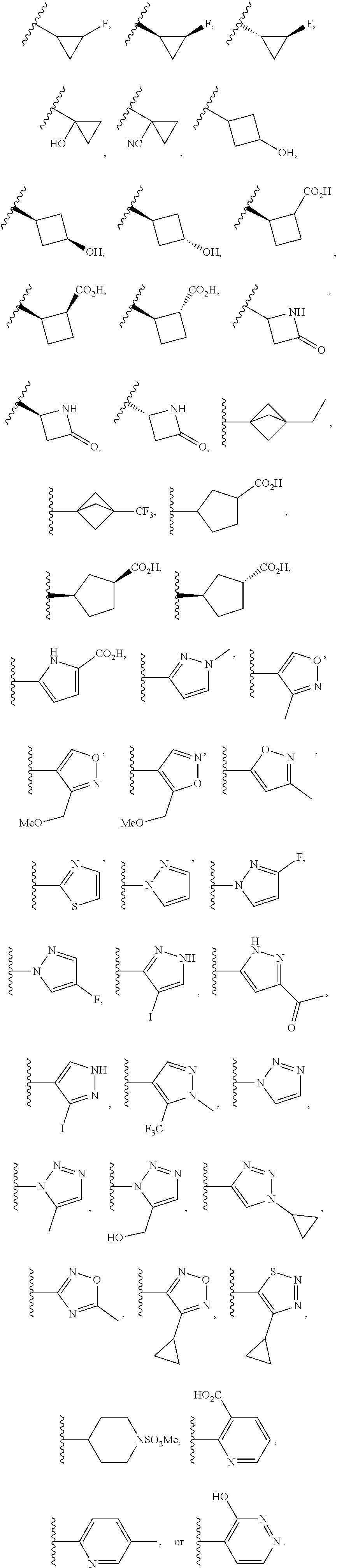

In some embodiments, Ring Y and its R^{y} substituents are

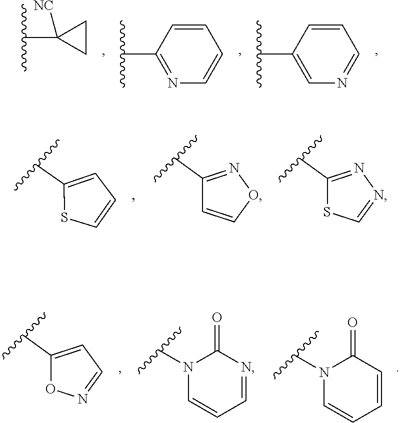

In some embodiments, Ring Y is

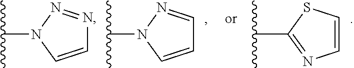

As described above and defined herein, R^{x}, and R^{y} are independently selected from hydrogen, R^{A}, halogen, —CN, —NO₂, —OR, —SR, —NR₂, —SiR₃, —S(O)R, —S(O)₂R, —S(O)(NR)R, —S(O)₂NR₂, —S(O)R, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R.

As described above and defined herein, each R^{w} is independently selected from hydrogen, R^{A}, R^{B′}, halogen, —CN, —NO₂, —OR, —SR, —NR₂, —SiR₃, —S(O)R, —S(O)₂R, —S(O)(NR)R, —S(O)₂NR₂, —S(O)R, —C(O)R, —C(S)R, —C(NR)R, —C(O)OR. —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R.

As described above and defined herein, each R^{w} of formula I-a′″ is independently selected from hydrogen, R^{A′}, R^{B′}, halogen, —CN, —NO₂, —OR, —SR, —NR₂, —SiR₃, —S(O)R, —S(O)₂R, —S(O)(NR)R, —S(O)₂NR₂, —S(O)R, —C(O)R, —C(O)OR, —C(O)NR₂, —C(O)NROR, —OC(O)R, —OC(O)NR₂, —P(O)R², —P(O)(OR)₂, —OP(O)R₂, —OP(O)(OR)₂, —NRC(O)OR, —NRC(O)R, —NRC(O)N(R)₂, and —NRS(O)₂R.

In some embodiments, each R^{w} is independently selected from hydrogen, R^{A}, halogen, —CN, —NO₂, —OR, —SR, —NR₂, —SiR₃, —S(O)R, —S(O)₂R, —S(O)(NR)R, —S(O)₂NR₂, —S(O)R, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R.

In some embodiments, each R^{w} is independently selected from R^{A}, R^{B′}, halogen, —CN, —NO₂, —OR, —SR, —NR₂, —SiR₃, —S(O)R, —S(O)₂R, —S(O)(NR)R₂, —S(O)₂NR₂, —S(O)R, —C(O)R, —C(S)R, —C(NR)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R.

In some embodiments, R^{w} is hydrogen. In some embodiments, R^{w} is R^{A}. In some embodiments, R^{w} is halogen. In some embodiments, R^{w} is —CN. In some embodiments, R^{w} is —NO₂. In some embodiments, R^{w} is —OR. In some embodiments, R^{w} is —SR. In some embodiments, R^{w} is —NR₂. In some embodiments, R^{w} is —SiR₃. In some embodiments, R^{w} is —S(O)₂R. In some embodiments, R^{w} is —S(O)₂NR₂. In some embodiments, R^{w} is —S(O)(NR)R. In some embodiments. R^{w} is —S(O)R. In some embodiments, R^{w} is —C(O)R. In some embodiments, R^{w} is —C(O)OR. In some embodiments. R^{w} is —C(O)NR₂. In some embodiments, R^{w} is —C(O)NROR. In some embodiments, R^{w} is —OC(O)R. In some embodiments, R^{w} is —OC(O)NR₂. In some embodiments, R^{w} is —P(O)R₂. In some embodiments, R^{w} is —P(O)(OR)₂. In some embodiments, R^{w} is —OP(O)R₂. In some embodiments, R^{w} is —OP(O)(OR)₂. In some embodiments, R^{w} is —OP(O)(OR)NR₂. In some embodiments, R^{w} is —OP(O)(NR₂)₂. In some embodiments, R^{w} is —NRC(O)OR. In some embodiments, R^{w} is —NRC(O)R. In some embodiments. R^{w} is —NRC(O)N(R)₂. In some embodiments. R^{w} is —NP(O)R₂. In some embodiments, R^{w} is —NRP(O)(OR)₂. In some embodiments, R^{w} is —NRP(O)(OR)NR₂. In some embodiments, R^{w} is —NRP(O)(NR₂)₂. In some embodiments, R^{w} is —NRS(O)₂R.

In some embodiments, R^{w}—C(O)OR. In some embodiments, R^{w} is —C(O)NR₂. In some embodiments, R^{w} is an optionally substituted phenyl. In some embodiments, R^{w} is an optionally substituted 5-6 membered heteroaryl ring having 1-4 heteroatoms independently selected from nitrogen, oxygen, and sulfur.

In some embodiments, R^{w} is fluoro, chloro, or bromo.

In some embodiments, R^{w} is —C(O)NHR. In some embodiments, R^{w} is —C(O)NHR, wherein R of R^{w} is optionally substituted C₁₋₆ aliphatic. In some embodiments, R^{w} is —C(O)NHR, wherein R of R^{w} is C₁₋₆ aliphatic, optionally substituted with —CN.

In some embodiments, R^{w} is optionally substituted C₁₋₆ aliphatic. In some embodiments, R^{w} is C₁₋₆ aliphatic, optionally substituted with —C(O)N(R^{∘})₂. In some embodiments, R^{w} is C₁₋₆ aliphatic, optionally substituted with —NR^{∘}(O)N(R^{∘})₂. In some embodiments. R^{w} is

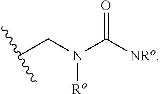

In some such embodiments. R^{∘} is hydrogen or C₁₋₆ aliphatic. In some embodiments. R^{w} is C₁₋₆ aliphatic optionally substituted with —OR^{∘}, wherein R^{∘} is hydrogen or C₁₋₆ aliphatic. In some embodiments, R^{w} is C₁₋₆ aliphatic optionally substituted with halogen (e.g., fluoro). In some embodiments, R^{w} is —CH₂F, —CHF₂, or —CF₃.

In some embodiments, R^{w} is C₁₋₆ aliphatic optionally substituted with halogen (e.g., fluoro). In some embodiments, R^{w} is

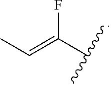 [SMILES: */C(F)=C/C ; "."]

In some embodiments, R^{w} is C₁₋₆ aliphatic optionally substituted with halogen (e.g., fluoro) or —NR^{∘}₂. In some embodiments, R^{w} is C₁₋₆ aliphatic optionally substituted with halogen (e.g., fluoro) and —NR^{∘}₂. In some embodiments, R^{w} is C₁₋₆ aliphatic optionally substituted with halogen (e.g., fluoro) or —NR^{∘}₂, wherein each R^{∘} is independently hydrogen or C₁₋₆ aliphatic. In some embodiments, R^{w} is C₁₋₆ aliphatic optionally substituted with halogen (e.g., fluoro) and —NR^{∘}₂, wherein each R^{∘} is independently hydrogen or C₁₋₆ aliphatic. In some embodiments, R^{w} is

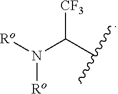

In some embodiments, R^{w} is C₁₋₆ aliphatic optionally substituted with —(CH₂)₀₋₄R^{∘}, wherein R^{∘} is a 3-6-membered saturated, partially unsaturated, or aryl ring having 0-4 heteroatoms independently selected from nitrogen, oxygen, or sulfur. In some embodiments, R^{w} is C₁₋₆ aliphatic optionally substituted with —(CH₂)₀₋₄R^{∘}, wherein R^{∘} is a C₁₋₆ aliphatic. In some embodiments, R^{w} is C₁₋₆ aliphatic optionally substituted with —R^{∘}, wherein R^{∘} is a 3-6-membered saturated, partially unsaturated, or aryl ring having 0-4 heteroatoms independently selected from nitrogen, oxygen, or sulfur. In some embodiments, R^{w} is C₁₋₆ aliphatic optionally substituted with —R^{∘}, wherein R^{∘} is a C₁₋₆ aliphatic.

In some embodiments, R^{w} is C₁₋₆ aliphatic optionally substituted with —(CH₂)₀₋₄R^{∘}, wherein R^{∘} is cyclopropyl, cyclobutyl, cyclopentyl, or cyclohexyl. In some embodiments, R^{w} is C₁₋₆ aliphatic optionally substituted with —R^{∘}, wherein R^{∘} is a cyclopropyl, cyclobutyl, cyclopentyl, or cyclohexyl.

In some embodiments, R^{∘} is an optionally substituted 3-7 membered saturated or partially unsaturated carbocyclic or heterocyclic ring having 1-2 heteroatoms independently selected from nitrogen, oxygen, and sulfur. In some embodiments, R^{w} is an optionally substituted 5-6 membered saturated or partially unsaturated carbocyclic or heterocyclic ring having 1-2 heteroatoms independently selected from nitrogen, oxygen, and sulfur.

In some embodiments R^{w} is an optionally substituted phenyl.

In some embodiments R^{w} is an optionally substituted 3-7 membered saturated or partially unsaturated carbocyclic. In some embodiments R^{w} is an optionally substituted 3-7 membered saturated or partially unsaturated carbocyclic. In some embodiments R^{w} is an optionally substituted cyclopropyl. In some embodiments R^{w} is an optionally substituted cyclobutyl. In some embodiments R^{w} is an optionally substituted cyclopentyl. In some embodiments R^{w} is an optionally substituted cyclohexyl. In some embodiments R^{w} is an optionally substituted cyclopropenyl. In some embodiments R^{w} is an optionally substituted cyclobutenyl. In some embodiments R^{w} is an optionally substituted cyclopentenyl. In some embodiments R^{w} is an optionally substituted cyclohexenyl.

In some embodiments R^{w} is an optionally substituted 3-7 membered saturated or partially unsaturated heterocyclic ring having 1-2 heteroatoms independently selected from nitrogen, oxygen, and sulfur. In some embodiments R^{w} is an optionally substituted 5-6 membered saturated or partially unsaturated heterocyclic ring having 1-2 heteroatoms independently selected from nitrogen, oxygen, and sulfur.

In some embodiments R^{w} is an optionally substituted 4 membered saturated or partially unsaturated heterocyclic ring having 1-2 heteroatoms independently selected from nitrogen, oxygen, and sulfur. In some embodiments R^{w} is an optionally substituted azetidinyl, oxetanyl, or thietanyl.

In some embodiments R^{w} is an optionally substituted 5 membered saturated or partially unsaturated heterocyclic ring having 1-2 heteroatoms independently selected from nitrogen, oxygen, and sulfur. In some embodiments, R^{w} is an optionally substituted pyrrolidinyl, pyrrolinyl, pyrazolidinyl, pyrazolinyl, imidazolidinyl, or imidazolinyl. In some embodiments, R^{w} is an optionally substituted dihydropyridinyl, pyrrolidinyl, dihydrofuranyl, tetrahydrofuranyl, dihydrothiophenyl, or tetrahydrothiophenyl.

In some embodiments R^{w} is an optionally substituted 6 membered saturated or partially unsaturated heterocyclic ring having 1-2 heteroatoms independently selected from nitrogen, oxygen, and sulfur. In some embodiments R^{w} is an optionally substituted piperidinyl, piperazinyl, tetrahydropyranyl, 2H-pyranyl, 4H-pyranyl, 1,4-dioxanyl, 1,4-dioxinyl, thianyl, 2H-thiopyranyl, 4H-thiopyranyl, 1,3-dithanyl, 1,4-dithanyl, morpholinyl, or thiomorpholinyl. In some embodiments, R^{w} is an optionally substituted dihydropyridinyl, tetrahydropyridinyl, dihydropyranyl, tetrahydropyranyl, dihydrothiopyranyl, or tetrahydrothiopyranyl.

In some embodiments, R^{w} is an optionally substituted

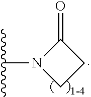

In some embodiments, R^{w} is an optionally substituted

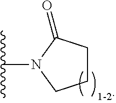

In some embodiments, R^{w} is an optionally substituted

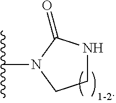

In some embodiments, R^{w} is

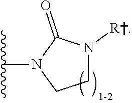

In some embodiments, R^{w} is optionally substituted

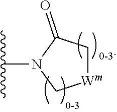

where W^{m} is O, S, C(O), or NR^{∘}, wherein R^{∘} is as described above and defined herein. In some embodiments, R^{w} is

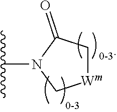

In some embodiments, R^{w} is optionally substituted

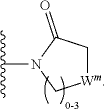

In some embodiments, R^{w} is

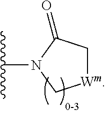

In some embodiments, R^{w} is

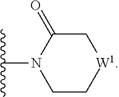

In some embodiments, R^{w} is

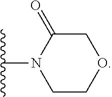 [SMILES: *N1CCOCC1=O]

In some embodiments, W^{m} is O. In some embodiments, W^{m} is S. In some embodiments, W^{m} is C(O). In some embodiments, W^{m} is NR^{∘}. In some embodiments, W^{m} is NR^{∘}, wherein R^{∘} is hydrogen or C₁₋₆ aliphatic.

In some embodiments, R^{w} is an optionally substituted 5-6 membered heteroaryl having 1-4 heteroatoms independently selected from nitrogen, oxygen, and sulfur. In some embodiments R^{w} is an optionally substituted 5 membered heteroaryl having 1-4 heteroatoms independently selected from nitrogen, oxygen, and sulfur. In some embodiments, R^{w} is an optionally substituted pyrazolyl, imidazolyl, triazolyl, or tetrazolyl. In some embodiments, R^{w} is an optionally substituted imidazolyl, optionally substituted with —C(O)N(R^{∘})₂. In some embodiments, R^{w} is an optionally substituted furanyl, thiophenyl, oxazolyl, isoxazolyl, isothiazolyl, thiazolyl, oxadiazolyl, or thiadiazolyl. In some embodiments, R^{w} is furanyl, optionally substituted with —C(O)N(R^{∘})₂.^{†}

In some embodiments R^{w} is an optionally substituted 6 membered heteroaryl having 1-4 heteroatoms independently selected from nitrogen, oxygen, and sulfur. In some embodiments R^{w} is an optionally substituted 6 membered heteroaryl having 1-4 nitrogen heteroatoms. In some embodiments, R^{w} is optionally substituted pyridinyl, pyrimidinyl, pyridazinyl, or triazinyl. In some embodiments, R^{w} is an optionally substituted pyridinonyl, pyrazinonyl, or pyrimidinoyl.

In some embodiments, R^{w} is —NHR, wherein R is an optionally substituted 5-6 membered heteroaryl having 1-4 heteroatoms independently selected from nitrogen, oxygen, and sulfur. In some embodiments, R^{w} is —NHR, wherein R is an optionally substituted 6 membered heteroaryl having 1-4 heteroatoms independently selected from nitrogen, oxygen, and sulfur.

In some embodiments, R^{w} is —S(O)₂NH₂. In some embodiments, R^{w} is —S(O)₂NHR, wherein R is an optionally substituted C₁₋₆ aliphatic. In embodiments, R^{w} is —S(O)₂NHR, wherein R is an optionally substituted phenyl, 4-7 membered saturated or partially unsaturated carbocyclic or heterocyclic ring having 1-2 heteroatoms independently selected from nitrogen, oxygen, and sulfur, or a 5-6 membered heteroaryl ring having 1-4 heteroatoms independently selected from nitrogen, oxygen, and sulfur.

In embodiments, R^{w} is —S(O)₂(NH)R. In some embodiments, R^{w} is —S(O)₂(NH)H. In some embodiments, R^{w} is —S(O)₂(NH)R, wherein R is an optionally substituted C₁₋₆ aliphatic. In some embodiments, R^{w} is —S(O)₂(NH)R, wherein R is an optionally substituted phenyl, 4-7 membered saturated or partially unsaturated carbocyclic or heterocyclic ring having 1-2 heteroatoms independently selected from nitrogen, oxygen, and sulfur, or a 5-6 membered heteroaryl ring having 1-4 heteroatoms independently selected from nitrogen, oxygen, and sulfur.

In some embodiments, R^{w} is —C(S)R, —C(NR)R, —S(O)R, —S(O)₂R, —S(O)(NR)R, —S(O)₂NR₂, —S(O)R, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, or —NRS(O)₂R. In some embodiments, R^{w} is —S(O)R, —S(O)₂R, —S(O)(NR)R, —S(O)₂NR₂, —S(O)R, —C(S)R, —C(NR)R, —C(O)R, —C(O)OR, —C(O)NR₂, —C(O)NROR, —OC(O)R, —OC(O)NR₂, —NRC(O)OR, —NRC(O)R, —NRC(O)N(R)₂, or —NRS(O)₂R. In some embodiments, R^{w} is —S(O)R, —S(O)₂R, —S(O)(NR)R₂, —S(O)₂NR₂, —S(O)R, or —NRS(O)₂R. In some embodiments, R^{w} is —C(O)R, —C(O)OR, —C(O)NR₂, —C(O)NROR, —OC(O)R, —OC(O)NR₂, —NRC(O)OR, —NRC(O)R, or —NRC(O)N(R)₂. In some embodiments, R^{w} is —S(O)R, —S(O)₂R, —S(O)(NR)R, —S(O)₂NR₂, —S(O)R, —C(S)R, —C(NR)R, —C(O)R, —C(O)OR, —C(O)NR₂, or —C(O)NROR. In some embodiments, R^{w} is —C(S)R, —C(NR)R, —C(O)R, —C(O)OR, or —C(O)NR₂.

In some embodiments, R^{w} is —C(O)H. In some embodiments, R^{w} is —C(O)R, wherein R is of R^{w} is optionally substituted C₁₋₆ aliphatic. In some embodiments, R^{w} is —C(O)R, wherein R is of R^{w} is optionally substituted phenyl. In some embodiments, R^{w} is —C(O)R, wherein R is of R^{w} is an optionally substituted 4-7 membered saturated or partially unsaturated heterocyclic having 1-2 heteroatoms independently selected from nitrogen, oxygen, and sulfur. In some embodiments, R^{w} is —C(O)R, wherein R is of R^{w} is an optionally substituted 5-6 membered heteroaryl ring having 1-4 heteroatoms independently selected from nitrogen, oxygen, and sulfur.

In some embodiments, R^{w} is —C(S)R. In some embodiments, R^{w} is —C(S)H. In some embodiments, R^{w} is —C(O)R, wherein R is of R^{w} is optionally substituted C₁₋₆ aliphatic. In some embodiments. R^{w} is —C(O)R, wherein R is of R^{w} is optionally substituted phenyl. In some embodiments, R^{w} is —C(O)R, wherein R is of R^{w} is an optionally substituted 4-7 membered saturated or partially unsaturated heterocyclic having 1-2 heteroatoms independently selected from nitrogen, oxygen, and sulfur. In some embodiments, R^{w} is —C(O)R, wherein R is of R^{w} is an optionally substituted 5-6 membered heteroaryl ring having 1-4 heteroatoms independently selected from nitrogen, oxygen, and sulfur.

In some embodiments, R^{w} is —C(O)OH. In some embodiments. R^{w} is —C(O)OR, wherein R is of R^{w} is optionally substituted C₁₋₆ aliphatic. In some embodiments, R^{w} is —C(O)OR, wherein R is of R^{w} is optionally substituted phenyl. In some embodiments, R^{w} is —C(O)OR, wherein R is of R^{w} is an optionally substituted 4-7 membered saturated or partially unsaturated heterocyclic having 1-2 heteroatoms independently selected from nitrogen, oxygen, and sulfur. In some embodiments, R^{w} is —C(O)OR, wherein R is of R^{w} is an optionally substituted 5-6 membered heteroaryl ring having 1-4 heteroatoms independently selected from nitrogen, oxygen, and sulfur.

In some embodiments, R^{w} is —C(O)NH₂. In some embodiments, R^{w} is —C(O)NR₂, wherein each R is of R^{w} is independently hydrogen or an optionally substituted C₁₋₆ aliphatic. In some embodiments, R^{w} is —C(O)NR₂, wherein each R is of R^{w} is independently hydrogen or an optionally substituted phenyl. In some embodiments, R^{w} is —C(O)NR₂, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, R^{w} is —C(O)NR₂, wherein each R is of R^{w} is independently hydrogen or an optionally substituted 5-6 membered heteroaryl ring having 1-4 heteroatoms independently selected from nitrogen, oxygen, and sulfur.

In some embodiments, R^{w} is —C(NR)R. In some embodiments, R^{w} is —C(NH)R. In some embodiments, R^{w} is C(NR)R, wherein each R is of R^{w} is independently hydrogen or an optionally substituted C₁₋₆ aliphatic. In some embodiments, R^{w} is C(NR)R, wherein each R is of R^{w} is independently hydrogen or an optionally substituted phenyl. In some embodiments, R^{w} is C(NR)R, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, R^{w} is C(NR)R, wherein each R is of R^{w} is independently hydrogen or an optionally substituted 5-6 membered heteroaryl ring having 1-4 heteroatoms independently selected from nitrogen, oxygen, and sulfur.

In some embodiments, R^{w} is —C(O)NHR, wherein R is an optionally substituted C₁₋₆ aliphatic. In some embodiments, R^{w} is —C(O)NHR, wherein R is C₁₋₆ aliphatic. In some embodiments. R^{w} is —C(O)NHR, wherein R is methyl, ethyl, or cyclopropyl. In some embodiments. R^{w} is —C(O)NR₂, wherein the two R groups of R^{w} are taken together with their intervening atoms to form a 3-10 membered saturated or partially unsaturated monocyclic, bicyclic, bridged bicyclic, or spirocyclic ring having 0-3 heteroatoms, in addition to the atom or adjacent atoms to which they are attached, independently selected form nitrogen, oxygen, and sulfur. In some embodiments, R^{w} is —C(O)NR₂, wherein the two R groups of R^{w} are taken together with their intervening atoms to form a 3-7 membered saturated or partially unsaturated monocyclic ring having 0-3 heteroatoms, in addition to the atom or adjacent atoms to which they are attached, independently selected form nitrogen, oxygen, and sulfur. In some embodiments, R^{w} is —C(O)NR₂, wherein the two R groups of R^{w} are taken together with their intervening atoms to form a 3-7 membered saturated or partially unsaturated monocyclic ring having 0 heteroatoms, in addition to the atom or adjacent atoms to which they are attached. In some embodiments, R^{w} is —C(O)NR₂, wherein the two R groups of R^{w} are taken together with their intervening atoms to form an aziridinyl, azetidinyl, diazetidinyl, pyrrolidinyl, or piperidinyl.

In some embodiments, R^{w} is —C(O)NROR, wherein each R is of R^{w} is independently hydrogen or an optionally substituted C₁₋₆ aliphatic. In some embodiments, R^{w} is —C(O)NROR, wherein each R is of R^{w} is independently hydrogen or an optionally substituted phenyl. In some embodiments, R^{w} is —C(O)NROR, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, R^{w} is —C(O)NROR, wherein each R is of R^{w} is independently hydrogen or an optionally substituted 5-6 membered heteroaryl ring having 1-4 heteroatoms independently selected from nitrogen, oxygen, and sulfur.

In some embodiments, R^{w} is —OC(O)H. In some embodiments. R^{w} is —OC(O)R, wherein R is of R^{w} is optionally substituted C₁₋₆ aliphatic. In some embodiments, R^{w} is —OC(O)R, wherein R is of R^{w} is optionally substituted phenyl. In some embodiments, R^{w} is —OC(O)R, wherein R is of R^{w} is an optionally substituted 4-7 membered saturated or partially unsaturated heterocyclic having 1-2 heteroatoms independently selected from nitrogen, oxygen, and sulfur. In some embodiments, R^{w} is —OC(O)R, wherein R is of R^{w} is an optionally substituted 5-6 membered heteroaryl ring having 1-4 heteroatoms independently selected from nitrogen, oxygen, and sulfur.

In some embodiments, R^{w} is —OC(O)NR₂, wherein each R is of R^{w} is independently hydrogen or an optionally substituted C₁₋₆ aliphatic. In some embodiments, R^{w} is —OC(O)NR₂, wherein each R is of R^{w} is independently hydrogen or an optionally substituted phenyl. In some embodiments, R^{w} is —OC(O)NR₂, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, R^{w} is —OC(O)NR₂, wherein each R is of R^{w} is independently hydrogen or an optionally substituted 5-6 membered heteroaryl ring having 1-4 heteroatoms independently selected from nitrogen, oxygen, and sulfur.

In some embodiments, R^{w} is —NRC(O)OR, wherein each R is of R^{w} is independently hydrogen or an optionally substituted C₁₋₆ aliphatic. In some embodiments, R^{w} is —NRC(O)OR, wherein each R is of R^{w} is independently hydrogen or an optionally substituted phenyl. In some embodiments, R^{w} is —NRC(O)OR, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, R^{w} is —NRC(O)OR, wherein each R is of R^{w} is independently hydrogen or an optionally substituted 5-6 membered heteroaryl ring having 1-4 heteroatoms independently selected from nitrogen, oxygen, and sulfur.

In some embodiments, R^{w} is —NRC(O)R, wherein each R is of R^{w} is independently hydrogen or an optionally substituted C₁₋₆ aliphatic. In some embodiments, R^{w} is —NRC(O)R, wherein each R is of R^{w} is independently hydrogen or an optionally substituted phenyl. In some embodiments, R^{w} is —NRC(O)R, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, R^{w} is —NRC(O)R, wherein each R is of R^{w} is independently hydrogen or an optionally substituted 5-6 membered heteroaryl ring having 1-4 heteroatoms independently selected from nitrogen, oxygen, and sulfur.

In some embodiments, R^{w} is —NRC(O)N(R)₂, wherein each R is of R^{w} is independently hydrogen or an optionally substituted C₁₋₆ aliphatic. In some embodiments, R^{w} is —NRC(O)N(R)₂, wherein each R is of R^{w} is independently hydrogen or an optionally substituted phenyl. In some embodiments, R^{w} is —NRC(O)N(R)₂, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, R^{w} is —NRC(O)N(R)₂, wherein each R is of R^{w} is independently hydrogen or an optionally substituted 5-6 membered heteroaryl ring having 1-4 heteroatoms independently selected from nitrogen, oxygen, and sulfur.

In some embodiments, R^{w} is —NRS(O)₂R, wherein each R is of R^{w} is independently hydrogen or an optionally substituted C₁₋₆ aliphatic. In some embodiments, R^{w} is —NRS(O)₂R, wherein each R is of R^{w} is independently hydrogen or an optionally substituted phenyl. In some embodiments, R^{w} is —NRS(O)₂R, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, R^{w} is —NRS(O)₂R, wherein each R is of R^{w} is independently hydrogen or an optionally substituted 5-6 membered heteroaryl ring having 1-4 heteroatoms independently selected from nitrogen, oxygen, and sulfur.

In some embodiments, R^{w} is —S(O)H. In some embodiments, R^{w} is —S(O)R, wherein R is of R^{w} is optionally substituted C₁₋₆ aliphatic. In some embodiments, R^{w} is —S(O)R, wherein R is of R is optionally substituted phenyl. In some embodiments, R^{w} is —S(O)R, wherein R is of R^{w} is an optionally substituted 4-7 membered saturated or partially unsaturated heterocyclic having 1-2 heteroatoms independently selected from nitrogen, oxygen, and sulfur. In some embodiments, R^{w} is —S(O)R, wherein R is of R^{w} is an optionally substituted 5-6 membered heteroaryl ring having 1-4 heteroatoms independently selected from nitrogen, oxygen, and sulfur.

In some embodiments, R^{w} is —S(O)₂H. In some embodiments, R^{w} is —S(O)₂R, wherein R is of R^{w} is optionally substituted C₁₋₆ aliphatic. In some embodiments, R^{w} is —S(O)₂R, wherein R is of R^{w} is optionally substituted phenyl. In some embodiments, R^{w} is —S(O)₂R, wherein R is of R^{w} is an optionally substituted 4-7 membered saturated or partially unsaturated heterocyclic having 1-2 heteroatoms independently selected from nitrogen, oxygen, and sulfur. In some embodiments, R^{w} is —S(O)₂R, wherein R is of R^{w} is an optionally substituted 5-6 membered heteroaryl ring having 1-4 heteroatoms independently selected from nitrogen, oxygen, and sulfur.

In some embodiments, R^{w} is —S(O)(NR)R, wherein each R is of R^{w} is independently hydrogen or optionally substituted C₁₋₆ aliphatic. In some embodiments, R^{w} is —S(O)(NR)R, wherein each R is of R is independently hydrogen or optionally substituted phenyl. In some embodiments. R^{w} is —S(O)(NR)R, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments, R^{w} is —S(O)(NR)R, wherein each R is of R^{w} is independently hydrogen or an optionally substituted 5-6 membered heteroaryl ring having 1-4 heteroatoms independently selected from nitrogen, oxygen, and sulfur.

In some embodiments, R^{w} is —S(O)₂NR₂, wherein each R is of R^{w} is independently hydrogen or an optionally substituted C₁₋₆ aliphatic. In some embodiments, R^{w} is —S(O)₂NR₂, wherein each R is of R^{w} is independently hydrogen or an optionally substituted phenyl. In some embodiments, R^{w} is —S(O)₂NR₂, wherein each R is of R^{w} is independently hydrogen or an optionally substituted 4-7 membered saturated or partially unsaturated heterocyclic having 1-2 heteroatoms independently selected from nitrogen, oxygen, and sulfur. In some embodiments. R^{w} is —S(O)₂NR₂, wherein each R is of R^{w} is independently hydrogen or an optionally substituted 5-6 membered heteroaryl ring having 1-4 heteroatoms independently selected from nitrogen, oxygen, and sulfur.

In some embodiments, R^{w} is an optionally substituted 3-7 membered saturated or partially unsaturated carbocyclic or heterocyclic ring having 1-2 heteroatoms independently selected from nitrogen, oxygen, and sulfur. In some embodiments, R^{w} is an optionally substituted 3-7 membered saturated or partially unsaturated heterocyclic ring having 1-2 heteroatoms independently selected from nitrogen, oxygen, and sulfur, and is substituted with ═O. In some embodiments, R^{w} is an optionally substituted 5-6 membered monocyclic heteroaryl ring having 1-4 heteroatoms independently selected from nitrogen, oxygen, and sulfur. In some embodiments, R^{w} is an optionally substituted 5-6 membered monocyclic heteroaryl ring having 1-4 heteroatoms independently selected from nitrogen, oxygen, and sulfur, and is substituted with ═O. In some embodiments. R^{w} is an optionally substituted:

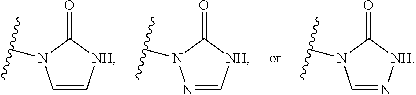 [SMILES: *n1cc[nH]c1=O ; *n1nc[nH]c1=O ; "or" ; *n1cn[nH]c1=O]

In some embodiments, R^{w} is:

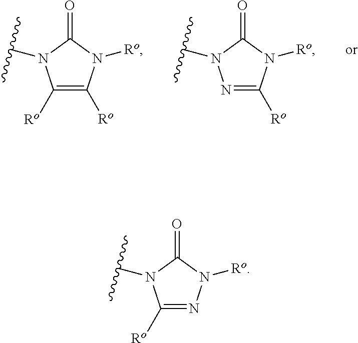

In some such embodiments, R^{∘} is hydrogen or C₁₋₆ aliphatic. In some embodiments, R^{w} is:

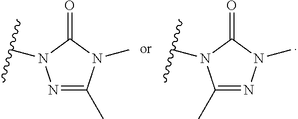

In some embodiments, R^{w} is an optionally substituted:

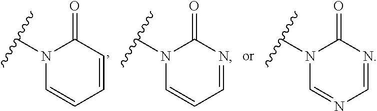

In some embodiments, R^{w} is:

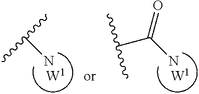

wherein Ring W¹ is an optionally substituted 3-7 membered saturated or partially unsaturated heterocyclic ring having 1 additional heteroatom selected from nitrogen, oxygen, and sulfur, or a 5-6 membered monocyclic heteroaryl ring having 1-3 additional heteroatoms independently selected from nitrogen, oxygen, and sulfur.

As defined above and described herein, Ring W¹ is an optionally substituted 3-7 membered saturated or partially unsaturated heterocyclic ring having 1 additional heteroatom selected from nitrogen, oxygen, and sulfur, or a 5-6 membered monocyclic heteroaryl ring having 1-3 additional heteroatoms independently selected from nitrogen, oxygen, and sulfur. In some embodiments, Ring W¹ is an optionally substituted 3-7 membered saturated or partially unsaturated heterocyclic ring having 1 additional heteroatom selected from nitrogen, oxygen, and sulfur. In some embodiments, Ring W¹ is an optionally substituted 5-6 membered saturated or partially unsaturated heterocyclic ring having 1 additional heteroatom selected from nitrogen, oxygen, and sulfur. In some embodiments, Ring W¹ is an optionally substituted 5-6 membered monocyclic heteroaryl ring having 1-3 additional heteroatoms independently selected from nitrogen, oxygen, and sulfur. In some embodiments, Ring W¹ is an optionally substituted 5 membered monocyclic heteroaryl ring having 1-3 additional heteroatoms independently selected from nitrogen, oxygen, and sulfur. In some embodiments, Ring W¹ is an optionally substituted 6 membered monocyclic heteroaryl ring having 1-3 additional heteroatoms independently selected from nitrogen, oxygen, and sulfur.

In some embodiments, R^{w} is:

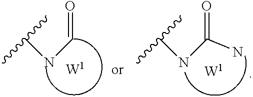

In some embodiments R^{w} is:

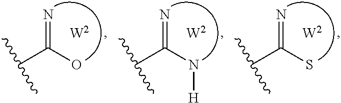

wherein Ring W² is an optionally substituted 3-7 membered partially unsaturated heterocyclic ring, or a 5-6 membered monocyclic heteroaryl ring having 1-2 additional heteroatoms independently selected from nitrogen, oxygen, and sulfur.

As defined above and described herein, Ring W² is an optionally substituted 3-7 membered partially unsaturated heterocyclic ring, or a 5-6 membered monocyclic heteroaryl ring having 1-2 additional heteroatoms independently selected from nitrogen, oxygen, and sulfur. In some embodiments, Ring W² is an optionally substituted 3-7 membered partially unsaturated heterocyclic ring. In some embodiments, Ring W² is an optionally substituted 5-6 membered partially unsaturated heterocyclic ring. In some embodiments, Ring W² is an optionally substituted 5-6 membered monocyclic heteroaryl ring having 1-2 additional heteroatoms independently selected from nitrogen, oxygen, and sulfur. In some embodiments, Ring W² is a 5 membered monocyclic heteroaryl ring having 1-2 additional heteroatoms independently selected from nitrogen, oxygen, and sulfur. In some embodiments, Ring W² is an optionally substituted oxazolyl, imidazolyl, thiazolyl, 1,3,4-thiadiazolyl, 2-imidazolinyl, 1,2,4-triazolyl, 1,2,4-oxadiazolyl, or 1,3,4-oxadiazolyl.

In some embodiments, R^{w} is an optionally substituted ring selected from:

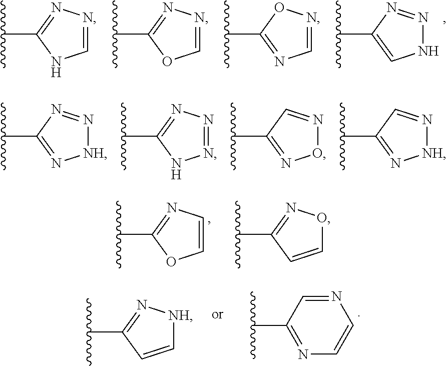

In some embodiments, R^{w} is:

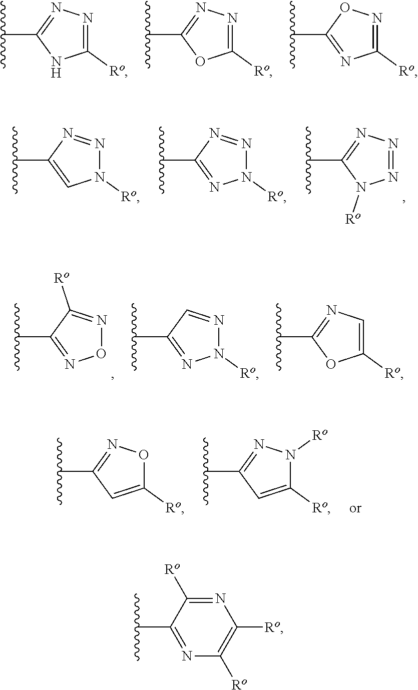

wherein each R^{∘} isa defined above and described herein (e.g., hydrogen or C₁₋₆ aliphatic.

In some embodiments, R^{w} is:

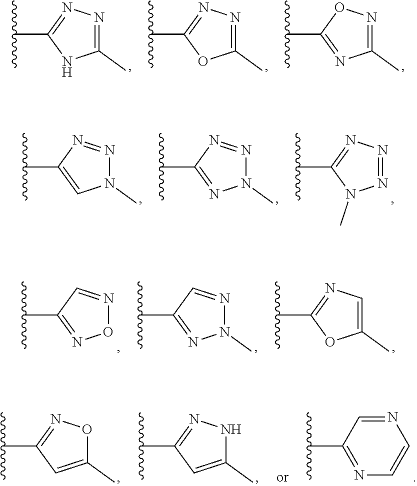

In some embodiments R is an optionally substituted 7-11 membered bicyclic heterocyclic ring having 1-4 heteroatoms independently selected from nitrogen, oxygen, and sulfur. In some embodiments R^{w} is an optionally substituted 9-10 membered bicyclic heterocyclic ring having 1-4 heteroatomns independently selected from nitrogen, oxygen, and sulfur. In some embodiments R^{w} is an optionally substituted benzo[d][1,3]dioxolyl. In some embodiments R^{w} is an optionally substituted an 11 membered bicyclic heterocyclic ring having 1-4 heteroatoms independently selected from nitrogen, oxygen, and sulfur.

In some embodiments R^{w} is an optionally substituted 8-11 membered bicyclic aryl or heteroaryl having 1-4 heteroatoms independently selected from nitrogen, oxygen and sulfur. In some embodiments, R^{w} is an optionally substituted naphthalenyl. In some embodiments, R^{w} is an optionally substituted dihydrobenzodioxepinyl. In some embodiments, R^{w} is an optionally substituted indenyl or dihydroindenyl.

In some embodiments, R^{w} is an optionally substituted 9-10 bicyclic heteroaryl having 1-4 heteroatoms independently selected from nitrogen, oxygen and sulfur.

In some embodiments R is an optionally substituted 9 bicyclic heteroaryl having 1-4 heteroatoms independently selected from nitrogen, oxygen and sulfur. In some embodiments R^{w} is an optionally substituted indolyl, benzofuranyl, benzothiophenyl, benzimidazolyl, benzoxazolyl, thienopyridinyl, pyrrolo[3,2-b]pyridinyl, pyrrolo[2,3,-b]pyridinyl, pyrazolyl[1,5-a]pyridinyl, or imidazo[1,2-a]pyridinyl, azaindazolyl (e.g., 4-, 5-, 6-, or 7-azaindazolyl), pyrrolol[2,3-c]pyridinyl or indolizinyl.

In some embodiments R^{w} is an optionally substituted 10 bicyclic heteroaryl having 1-4 heteroatoms independently selected from nitrogen, oxygen and sulfur. In some embodiments R^{w} is an optionally substituted quinolinyl, isoquinolinyl, quinolizinyl, quinoxalinyl, phthalazinyl, quinazolinyl, cinnolinyl, or 1,8-naphthyridinyl.

In some embodiments, R^{w} is R^{B′}. In some embodiments, R^{w} is R^{A′}.

In some embodiments, R^{w} is fluoro, chloro, —CN, methyl, —CF₃, —CHF₂, —OH, —OMe, —OCH₂CO₂Me, —CO₂H, —C(O)NH₂, —C(O)NHMe, —C(O)NHEt, —C(O)NHnPr, —C(O)NHCH₂CH₂OH, —C(O)NMe₂, —C(O)N(Me)Et, —C(O)N(Me)nPr, —CH₂NHMe,

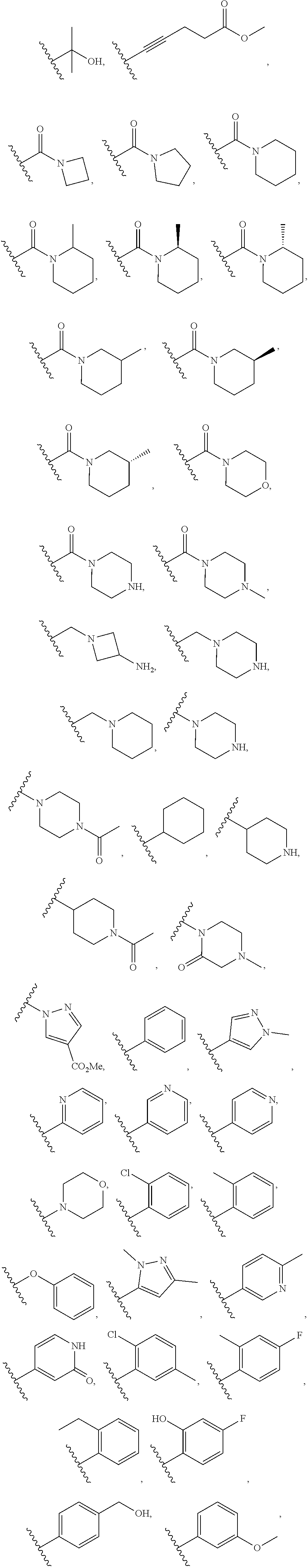

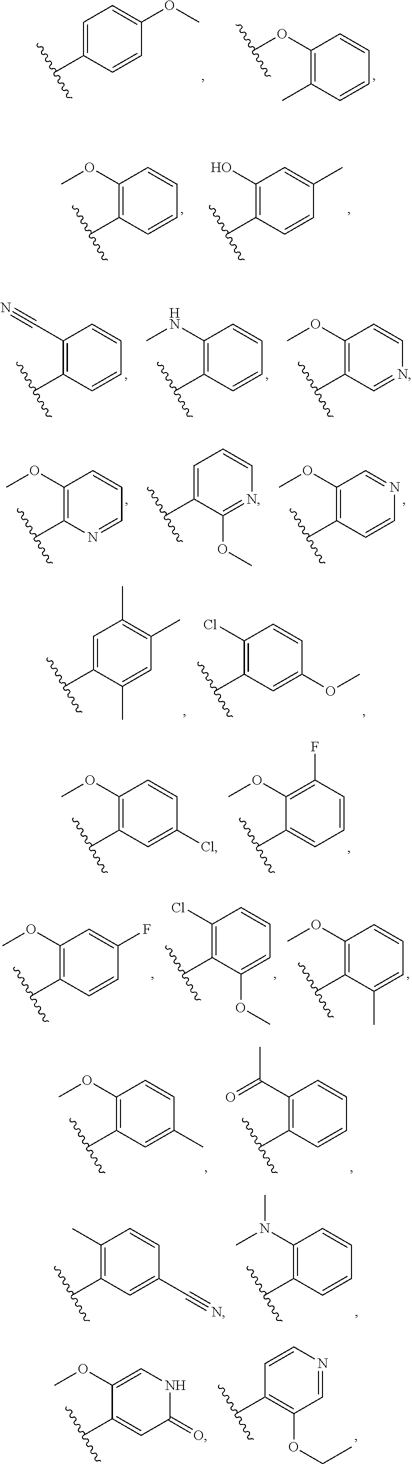

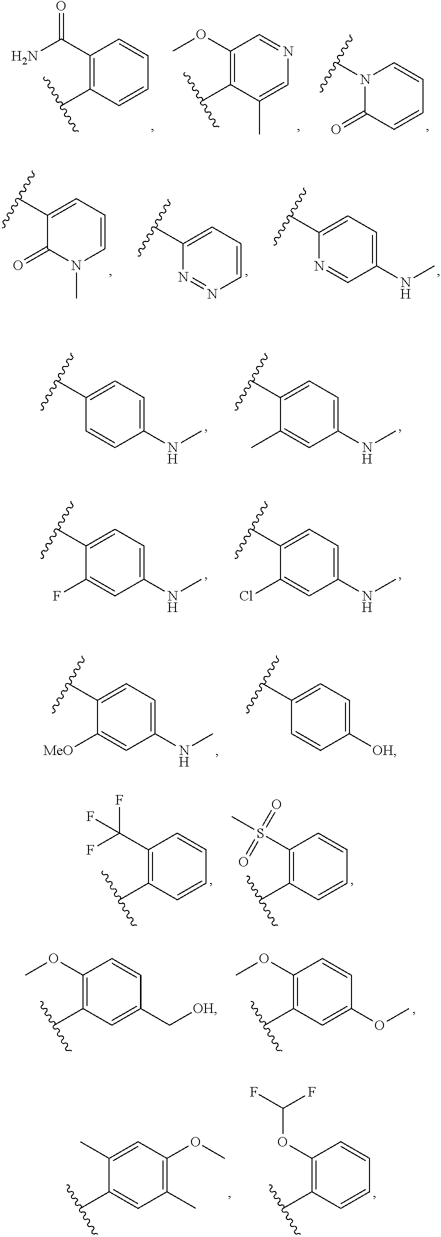

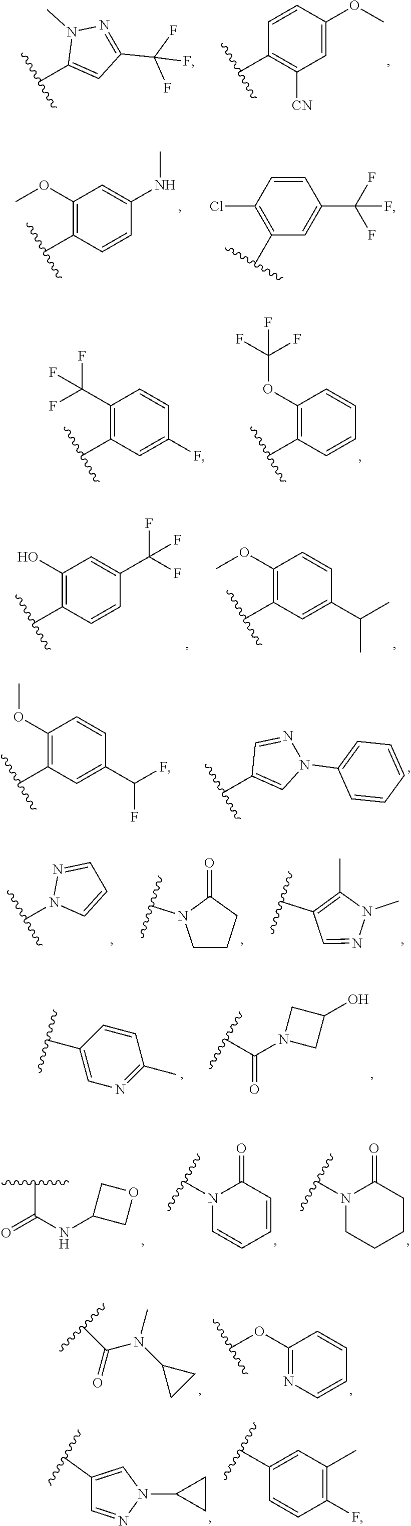

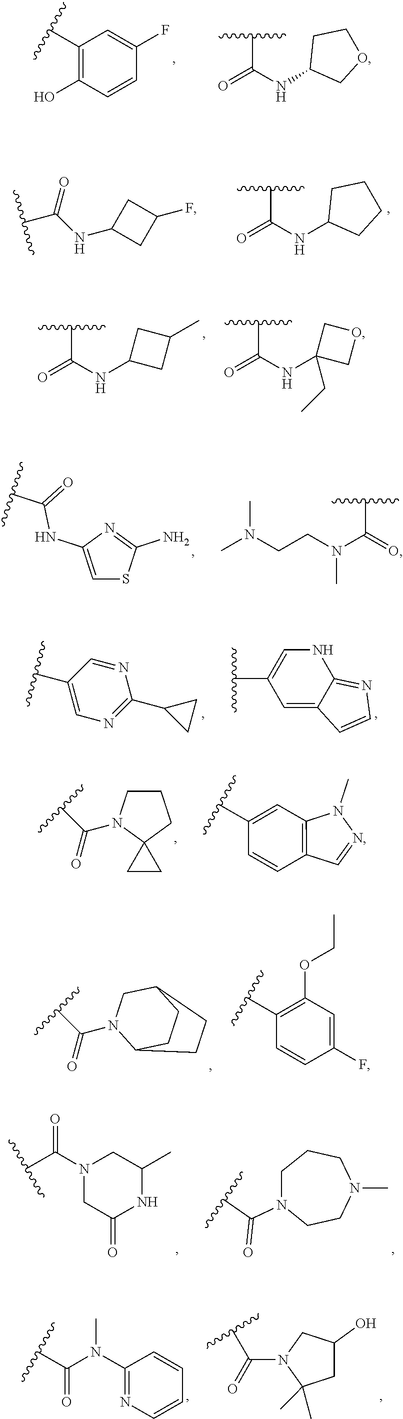

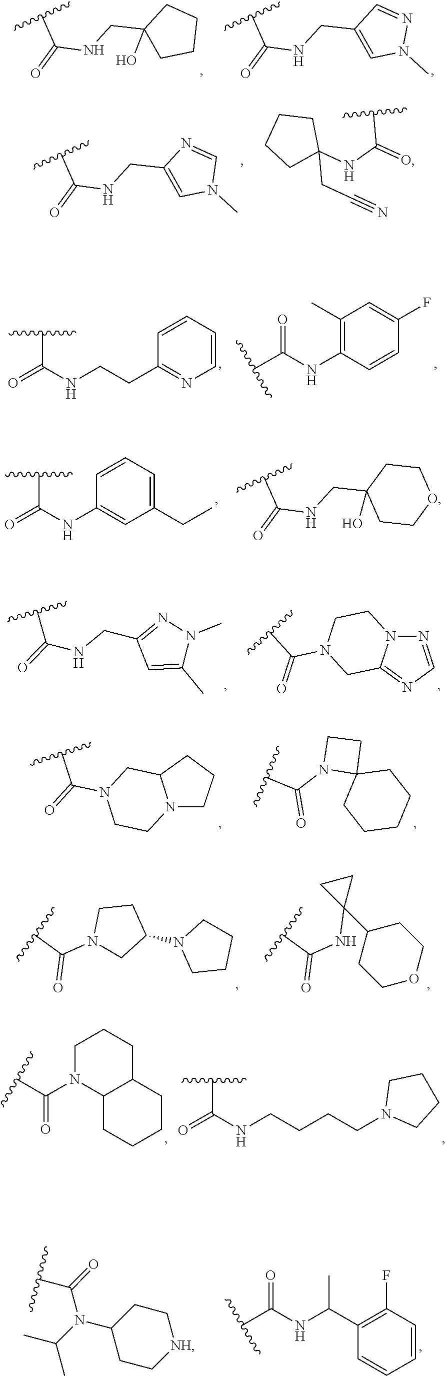

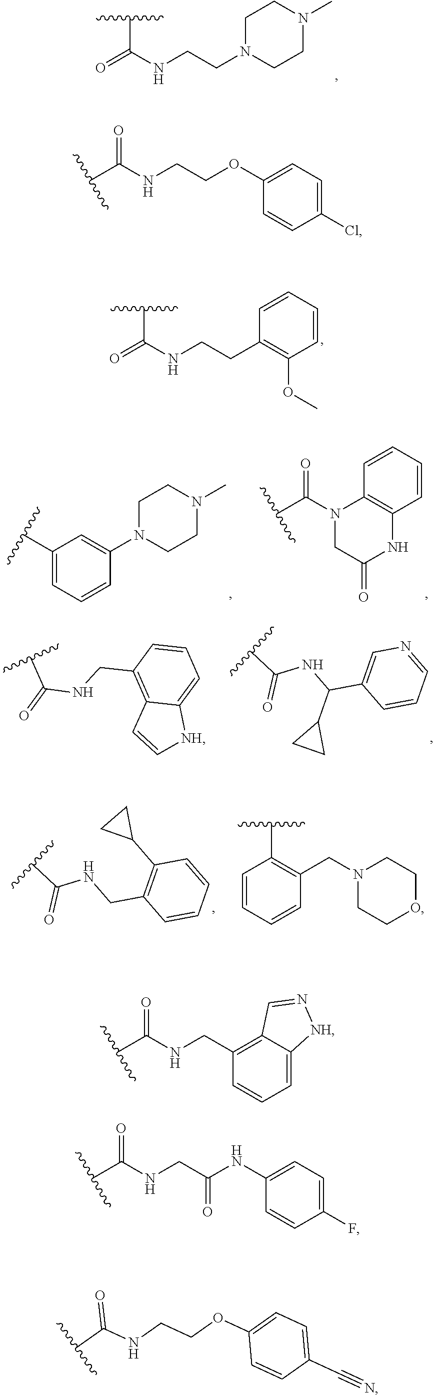

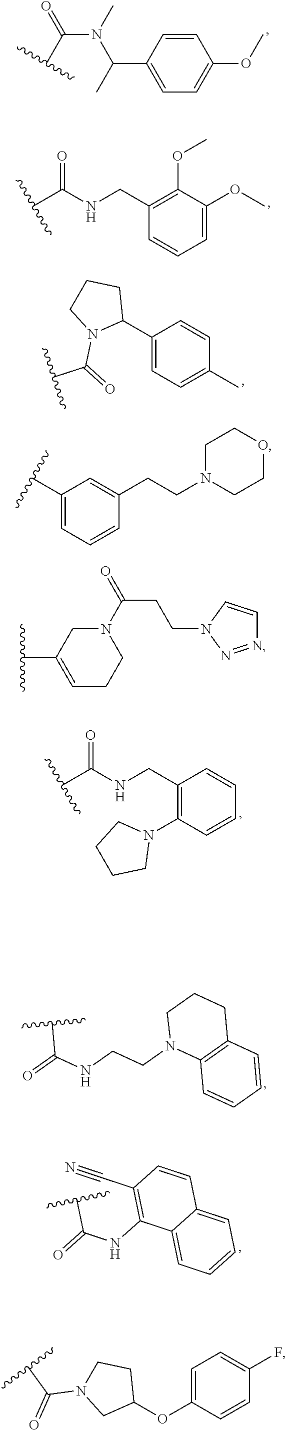

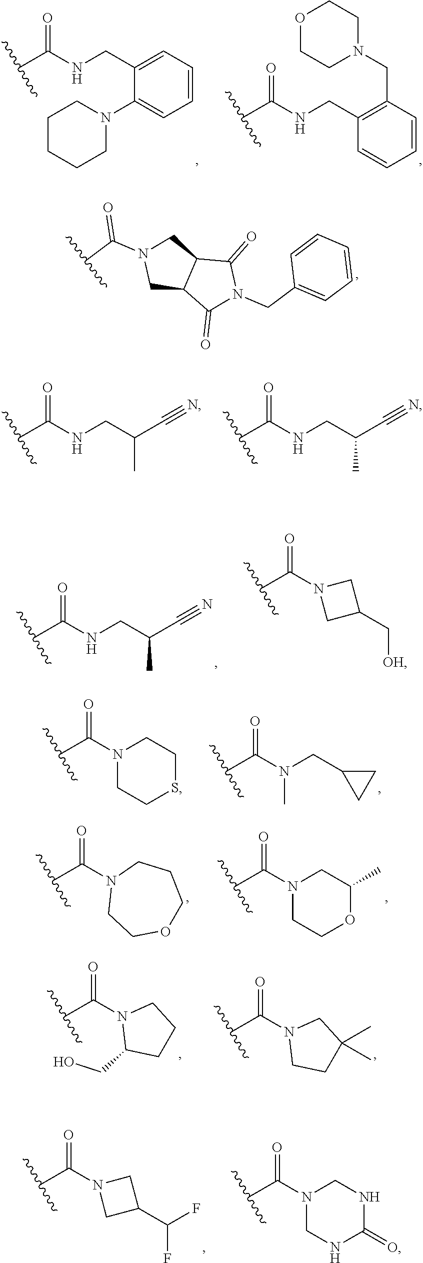

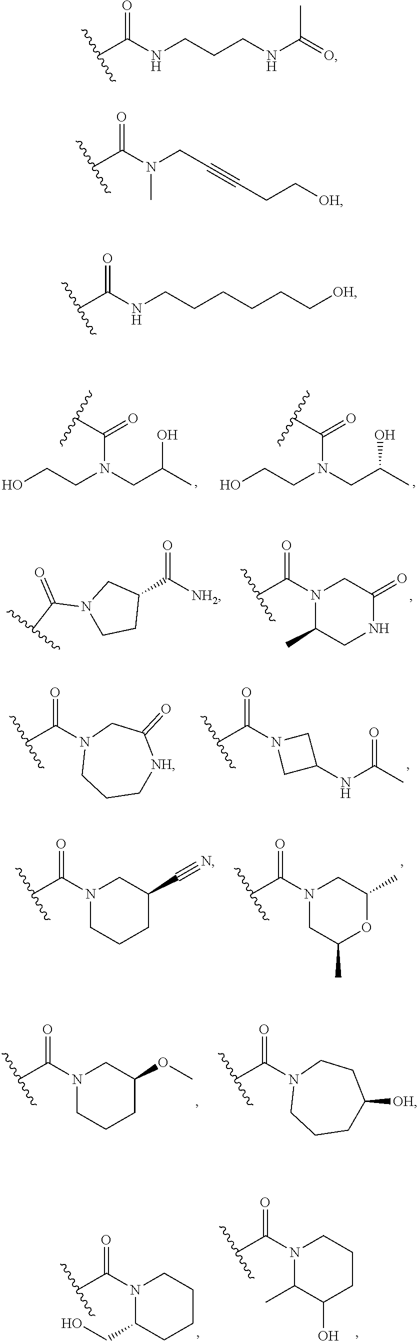

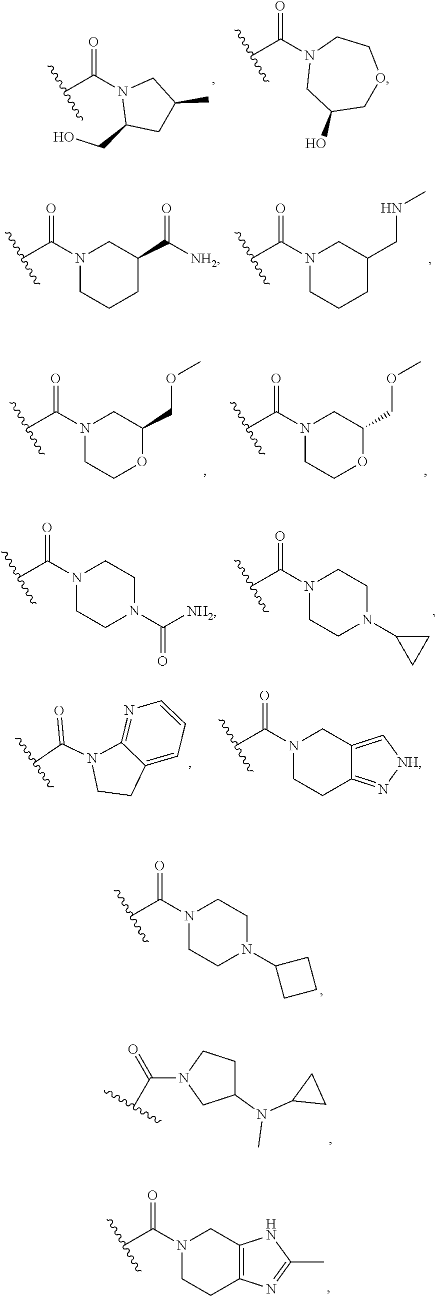

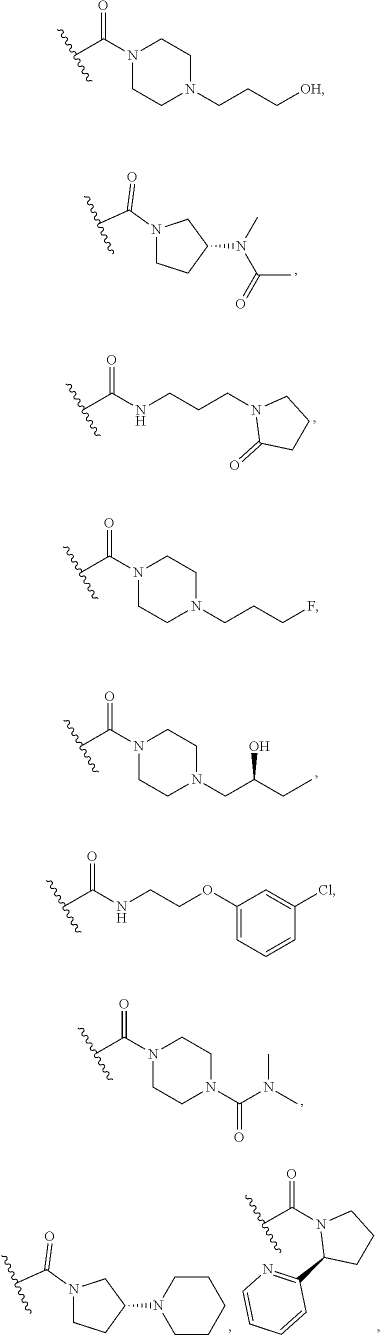

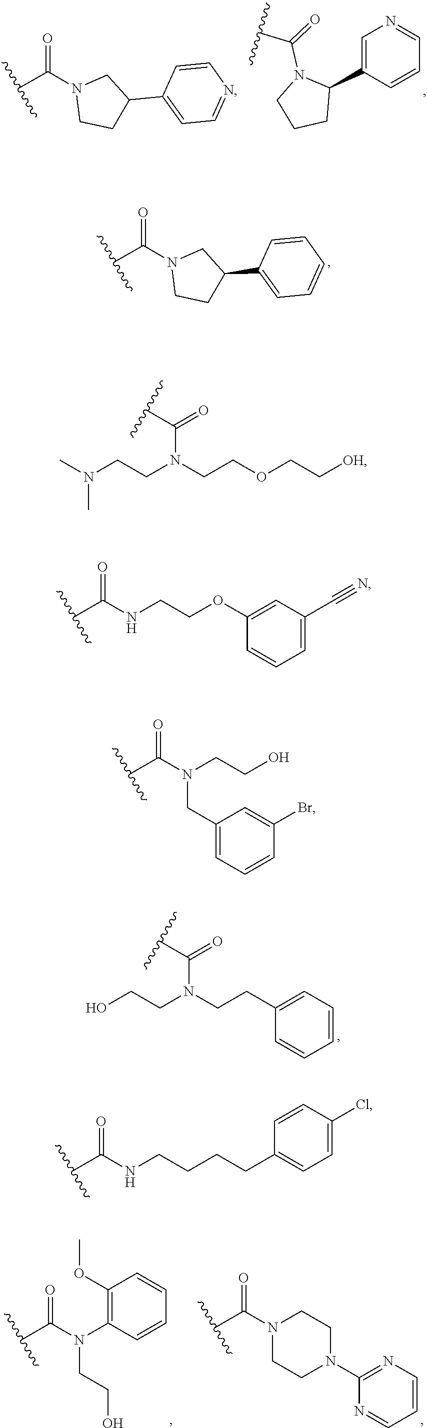

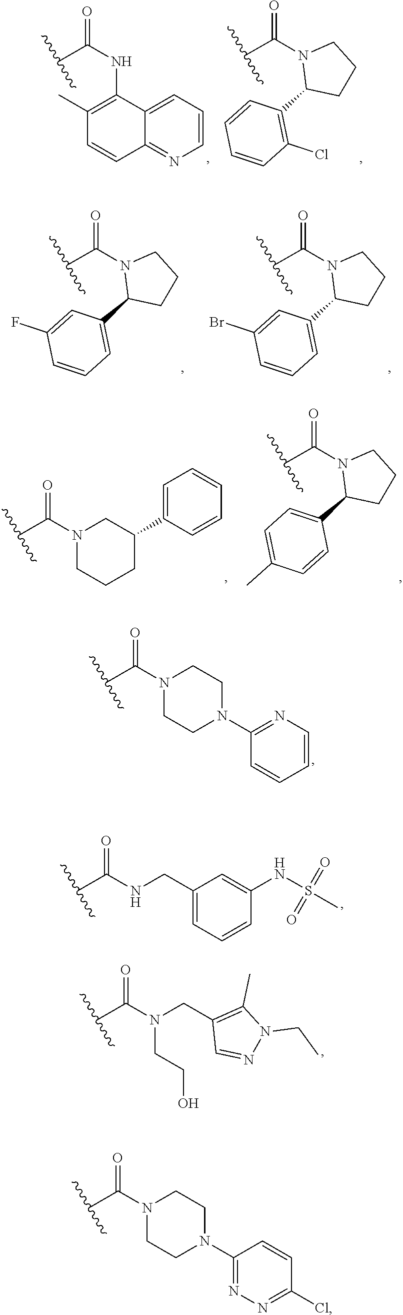

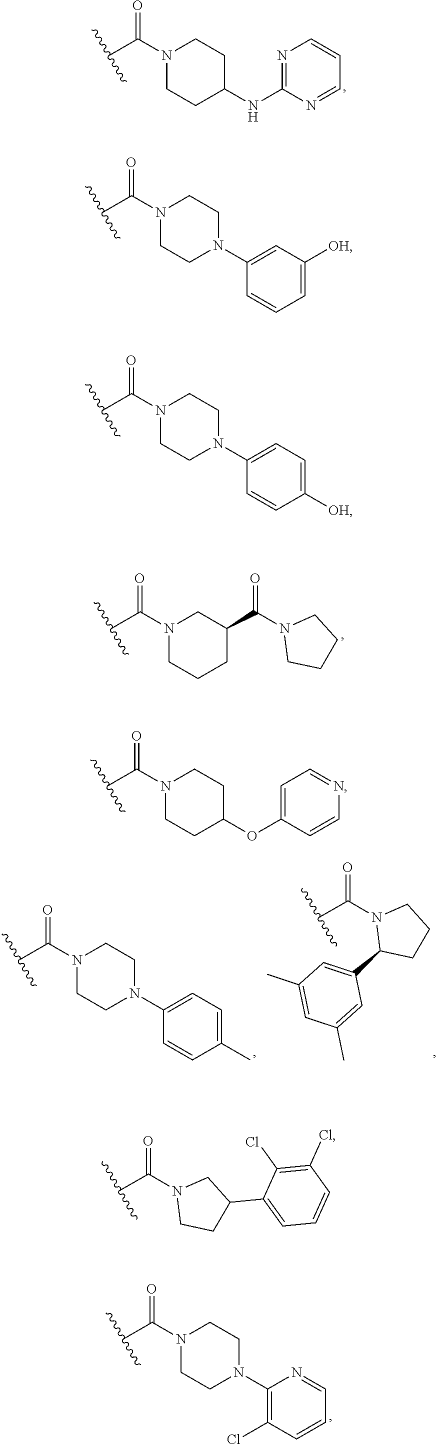

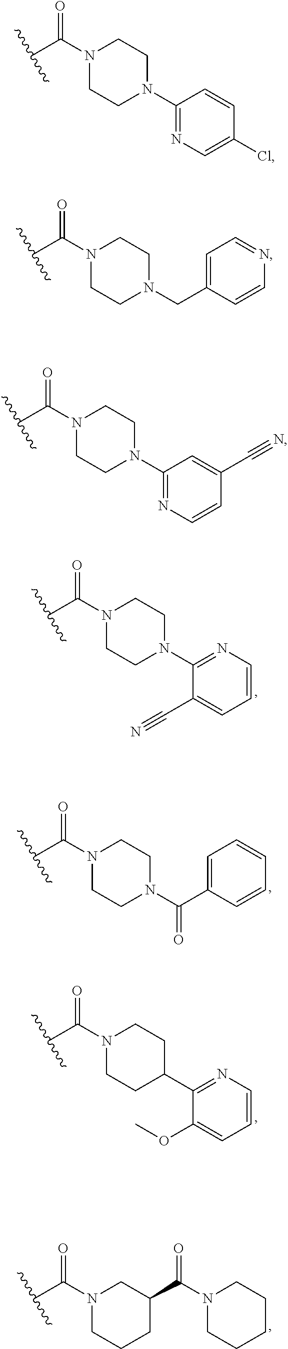

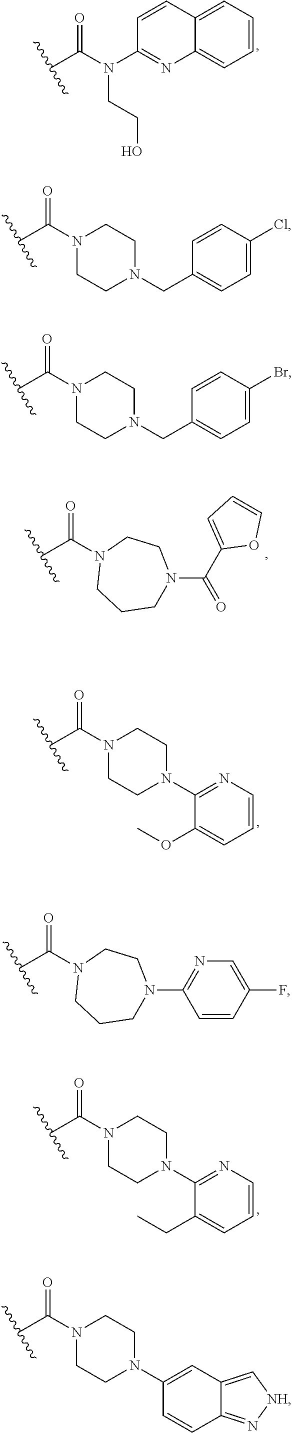

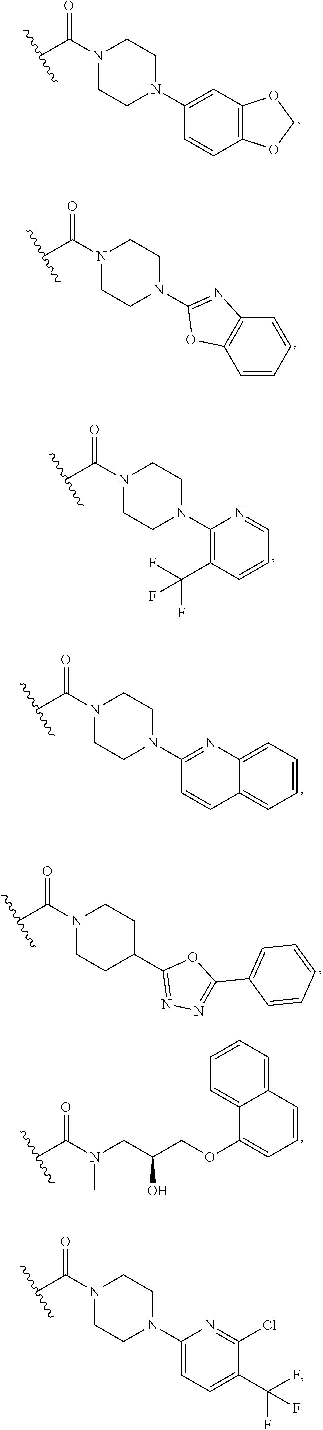

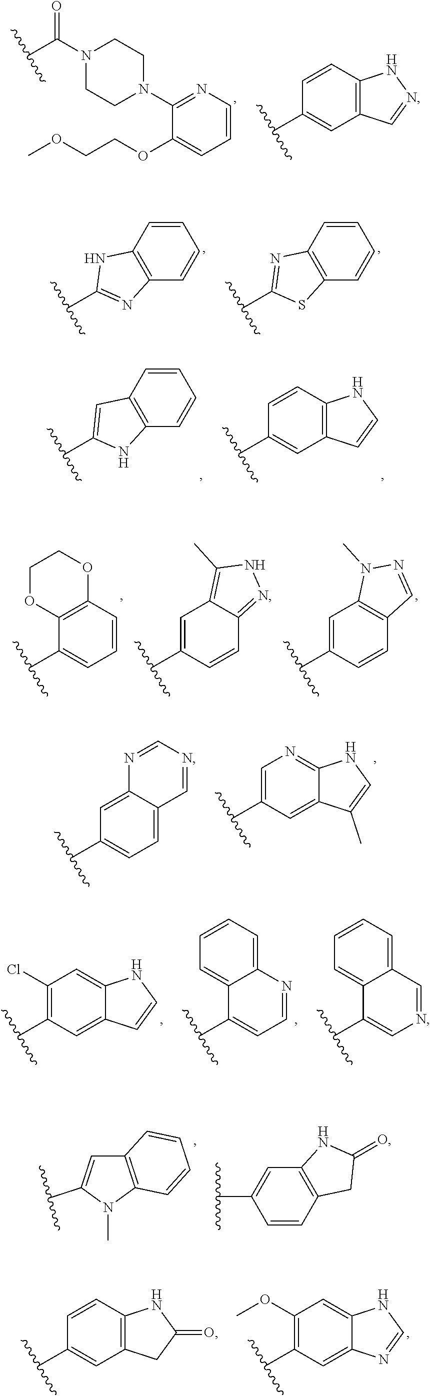

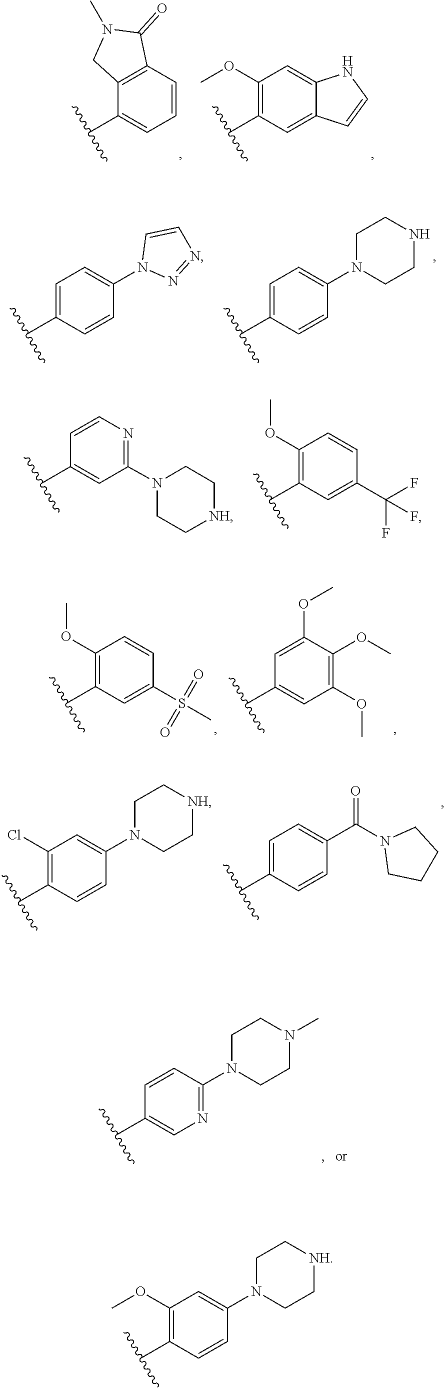

In some embodiments, R^{w} is

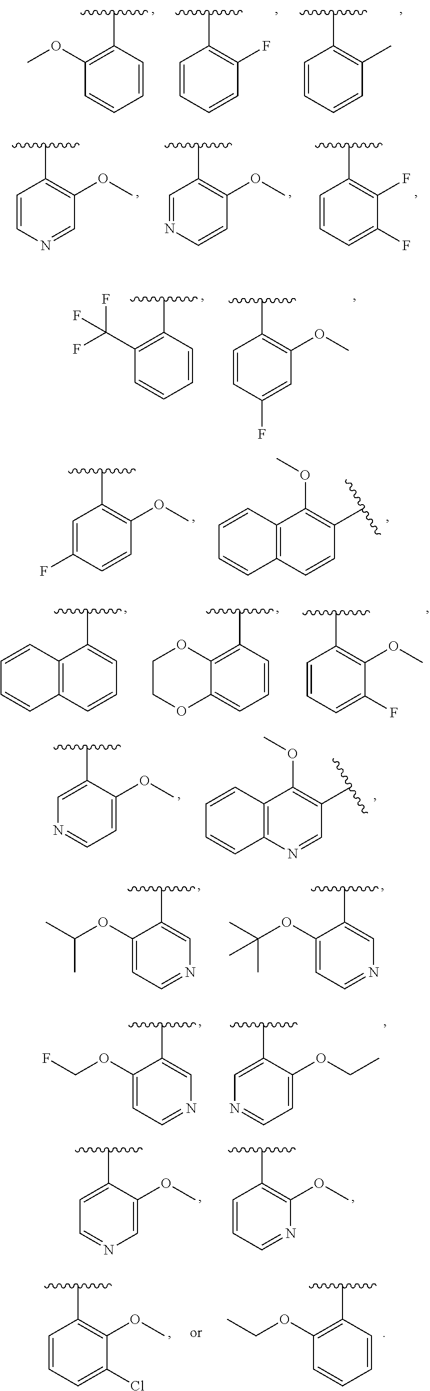

In some embodiments, R^{w} is S(O)₂NH₂, —S(O)₂N(CH₃)₂, —S(O)(NH)CH₃,

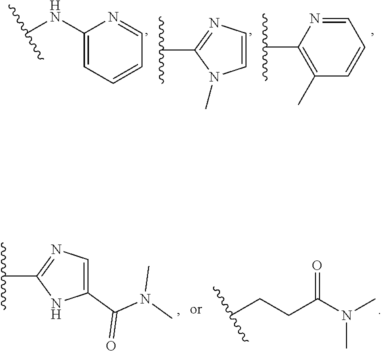

In some embodiments, R^{w} is

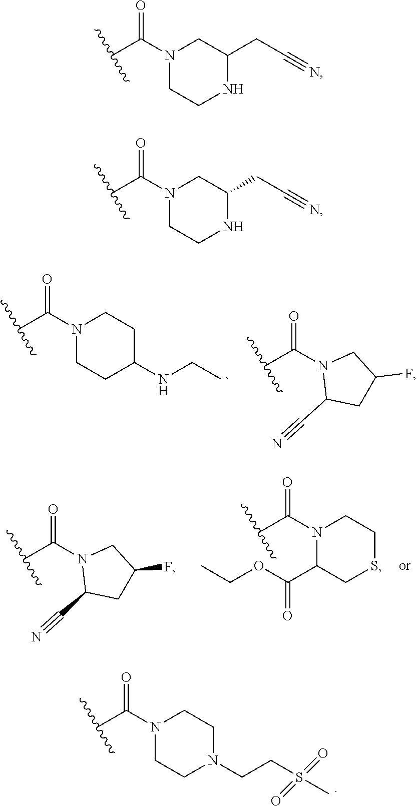

In some embodiments, R^{w} is

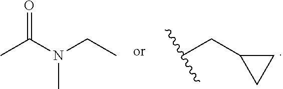

In some embodiments, R^{x} is selected from hydrogen, R^{A}, halogen, —CN, —NO₂, —OR, —SR, —NR₂, —SiR₃, —S(O)R, —S(O)₂R, —S(O)(NR)R, —S(O)₂NR₂, —S(O)R, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)N(R)₂, and —NRS(O)₂R.

In some embodiments, R^{x} is hydrogen. In some embodiments, R^{x} is R^{A}. In some embodiments, R^{x} is halogen. In some embodiments, R^{x} is —CN. In some embodiments, R^{w} is —NO₂. In some embodiments, R^{x} is —OR. In some embodiments, R^{x} is —SR. In some embodiments. R^{x} is —NR₂. In some embodiments, R^{w} is —SiR₃. In some embodiments, R^{x} is —S(O)₂R. In some embodiments, R^{w} is —S(O)₂NR₂. In some embodiments, R^{x} is —S(O)(NR)R. In some embodiments, R^{x} is —S(O)R. In some embodiments, R^{x} is —C(O)R. In some embodiments, R is —C(O)OR. In some embodiments, R^{X} is —C(O)NR₂. In some embodiments, R^{X} is —C(O)NROR. In some embodiments, R^{X} is —OC(O)R. In some embodiments, R^{x} is —OC(O)NR₂. In some embodiments, R^{X} is —P(O)R₂. In some embodiments, R^{X} is —P(O)(OR)₂. In some embodiments, R^{x} is —OP(O)R₂. In some embodiments, R^{X} is —OP(O)(OR)₂. In some embodiments, R^{x} is —OP(O)(OR)NR₂. In some embodiments, R is —OP(O)(NR₂)₂. In some embodiments, R^{w} is —NRC(O)OR. In some embodiments. R is —NRC(O)R. In some embodiments, RN is —NRC(O)N(R)₂. In some embodiments, R^{x} is —NP(O)R₂. In some embodiments, R is —NRP(O)(OR)₂. In some embodiments, R^{x} is —NRP(O)(OR)NR₂. In some embodiments, R^{w} is —NRP(O)(NR₂)₂. In some embodiments, R^{w} is —NRS(O)₂R.

In some embodiments, each R^{x} is independently selected from R^{A}, halogen, —CN, —NO₂, —OR, —SR, —NR₂, —SiR₃, —S(O)R, —S(O)₂R, —S(O)(NR)R, —S(O)₂NR₂, —S(O)R, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R

In some embodiments, R^{x} is optionally substituted C₁₋₆ aliphatic. In some embodiments, R^{x} is C₁₋₆ aliphatic optionally substituted with —OR^{∘}, wherein R^{∘} is hydrogen or C₁₋₆ aliphatic.

In some embodiments, R^{x} is —CO₂Me, —CH₂OH, —C(O)Me, —C(O)Et, —C(O)iPr, —C(O)cyclopropyl, —C(O)oxetanyl, —C(O)tetrahydropyranyl, or pyridyl.

In some embodiments, RN is fluoro. In some embodiments, R^{x} is —S(O)₂CH₃. In some embodiments, RN is —C(O)N(CH₃)₂.

In some embodiments, R^{w} is —C(O)R or —C(O)OR. In some embodiments, R^{X} is —C(O)R.

In some embodiments, R^{x} is —C(O)R, wherein R of R^{x} is optionally substituted C₁₋₆ aliphatic. In some embodiments, R is —C(O)R, wherein R of R is C₁₋₆ aliphatic. In some embodiments, R^{w} is —C(O)R, wherein R of R is methyl, ethyl, n-propyl, or isopropyl. In some embodiments, R^{x} is —C(O)R, wherein R of R is C₁₋₆ aliphatic substituted with —OR^{∘} or —C(O)OR^{∘}, wherein R^{∘} is hydrogen or C₁₋₆ aliphatic. In some embodiments, R is —C(O)R, wherein R of R is C₁₋₆ aliphatic substituted with —OR^{∘}, wherein R^{∘} is hydrogen or C₁₋₆ aliphatic. In some embodiments, R^{x} is —C(O)R, wherein R of R is C₁₋₆ aliphatic substituted with —C(O)OR^{∘}, wherein R^{∘} is hydrogen or C₁₋₆ aliphatic.

In some embodiments, R^{x} is

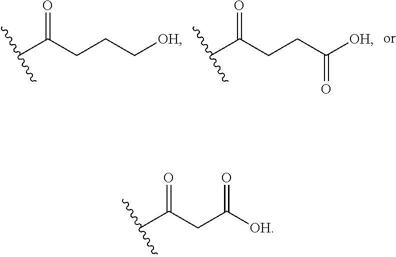 [SMILES: *C(=O)CCCO ; *C(=O)CCC(=O)O ; "or" ; *C(=O)CC(=O)O]

In some embodiments, R^{X} is —C(O)R, wherein R of R^{X} is C₁₋₆ aliphatic optionally substituted with —C(O)OR^{∘}, —OR^{∘}, halogen (e.g., fluoro), or ═O. In some embodiments, R^{X} is —C(O)OR. In some embodiments, R^{X} is —C(O)OR, wherein R of R^{X} is an optionally substituted C₁₋₆ aliphatic.

In some embodiments, R^{x} is

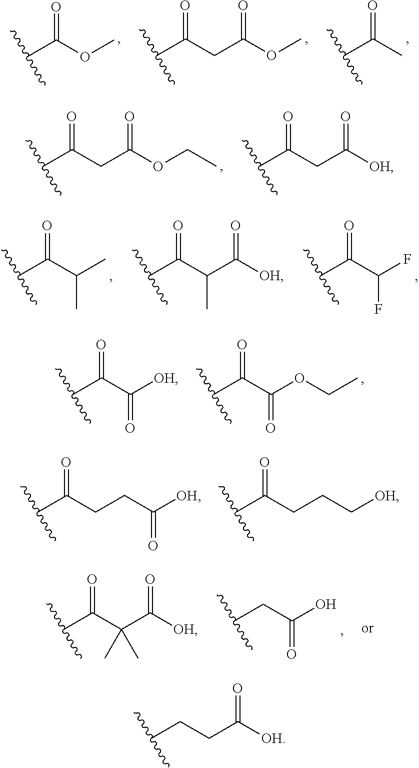

In some embodiments, each R^{y} is independently selected from R^{A}, halogen, —CN, —NO₂, —OR, —SR, —NR₂, —SiR₃, —S(O)R, —S(O)₂R, —S(O)(NR)R, —S(O)₂NR₂, —S(O)R, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R.

In some embodiments, R^{y} is hydrogen. In some embodiments. R is R^{A}. In some embodiments, R^{y} is halogen. In some embodiments, R^{y} is —CN. In some embodiments, R^{y} is —NO₂. In some embodiments, R^{y} is —OR. In some embodiments, R^{y} is —SR. In some embodiments, R^{y} is —NR₂. In some embodiments. R^{y} is —SiR₃. In some embodiments, R is —S(O)₂R. In some embodiments, R is —S(O)₂NR₂. In some embodiments, R^{y} is —S(O)(NR)R. In some embodiments, R^{y} is —S(O)R. In some embodiments, R^{y} is —C(O)R. In some embodiments, R^{y} is —C(O)OR. In some embodiments, R^{w} is —C(O)NR₂. In some embodiments, R is —C(O)NROR. In some embodiments. R is —OC(O)R. In some embodiments, R is —OC(O)NR₂. In some embodiments, R^{y} is —P(O)R₂. In some embodiments, R is —P(O)(OR)₂. In some embodiments, R^{y} is —OP(O)R₂. In some embodiments, R is —OP(O)(OR)₂. In some embodiments, R^{y} is —OP(O)(OR)NR₂. In some embodiments, R^{y} is —OP(O)(NR₂)₂. In some embodiments, R^{y} is —NRC(O)OR. In some embodiments, R^{y} is —NRC(O)R. In some embodiments, R^{y} is —NRC(O)N(R)₂. In some embodiments, R^{y} is —NP(O)R₂. In some embodiments, R^{y} is —NRP(O)(OR)₂. In some embodiments, R^{y} is —NRP(O)(OR)NR₂. In some embodiments, R^{y} is —NRP(O)(NR₂)₂. In some embodiments, R^{y} is —NRS(O)₂R.

In some embodiments, R^{y} is fluoro, chloro, bromo, iodo, methyl, ethyl, cyclopropyl, —CF₃, —CN, CH₂O, —CO₂H, —CO₂Me, —CO₂tBu, —C(O)Me, —NH₂, —NHMe, —NHAc, —NHC(O)Et, —OH, —OMe, —OCH₂CH₂NH₂, —CH₂OH, —CH₂OMe, —CH₂NHMe, —CH₂NHAc, —CH₂SO₂Me, —SO₂Me, —SO₂NH₂, —SO₂NHME,

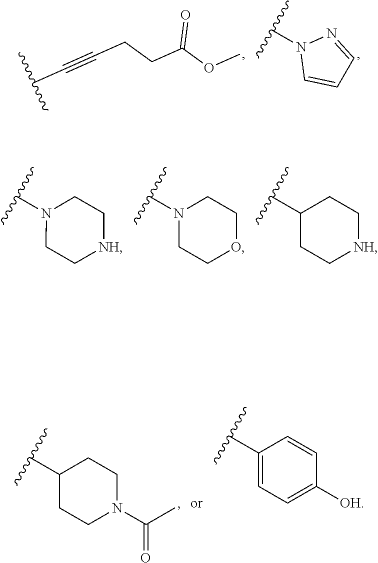

In some embodiments, R^{y} is —C(O)H.

In some embodiment, R^{w}, R^{x}, and R^{y} are as depicted in the compounds of Table 1, below.

As described above and defined herein, each R^{A′} is independently R^{A} or an 8-10 membered monocyclic or bicyclic aryl or heteroaryl with 1-4 heteroatoms independently selected from nitrogen, oxygen and sulfur. In some embodiments, R^{A′} is R^{A}. In some embodiments, R^{A′} is an 8-10 membered monocyclic or bicyclic aryl or heteroaryl with 1-4 heteroatoms independently selected from nitrogen, oxygen and sulfur. In some embodiments, R^{A′} is optionally substituted naphthalenyl or dihydrobenzo[b][1,4]dioxinyl.

As described above and defined herein, each R^{A} is independently an optionally substituted group selected from C₁₋₆ aliphatic, phenyl, naphthalenyl, a 3-7 membered saturated or partially unsaturated carbocyclic or heterocyclic ring having 1-2 heteroatoms independently selected from nitrogen, oxygen, and sulfur, a 5-10 membered monocyclic or bicyclic aryl or heteroaryl ring having 1-4 heteroatoms independently selected from nitrogen, oxygen, and sulfur, or a 7-11 membered bicyclic heterocyclic ring having 1-4 heteroatoms independently selected from nitrogen, oxygen, and sulfur. In some embodiments, each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-10 membered monocyclic or bicyclic heteroaryl ring having 1-4 heteroatoms independently selected from nitrogen, oxygen, and sulfur. In some embodiments, each R^{A} is independently an optionally substituted group selected from C₁₋₆ aliphatic, phenyl, naphthalenyl, a 3-7 membered saturated or partially unsaturated carbocyclic or heterocyclic ring having 1-2 heteroatoms independently selected from nitrogen, oxygen, and sulfur, or a 5-10 membered monocyclic or bicyclic heteroaryl ring having 1-4 heteroatoms independently selected from nitrogen, oxygen, and sulfur.

In some embodiments, each R^{A} is independently selected from C₁₋₆ aliphatic, phenyl, naphthalenyl, a 3-7 membered saturated or partially unsaturated carbocyclic or heterocyclic ring having 1-2 heteroatoms independently selected from nitrogen, oxygen, and sulfur, a 5-10 membered monocyclic or bicyclic aryl or heteroaryl ring having 1-4 heteroatoms independently selected from nitrogen, oxygen, and sulfur, or a 7-11 membered bicyclic heterocyclic ring having 1-4 heteroatoms independently selected from nitrogen, oxygen, and sulfur. In some embodiments, each R^{A} is independently an optionally substituted group selected from C₁₋₆ aliphatic, phenyl, naphthalenyl, a 3-7 membered saturated or partially unsaturated carbocyclic, a 3-7 membered saturated heterocyclic ring having 1-2 heteroatoms independently selected from nitrogen, oxygen, and sulfur, a 5-10 membered monocyclic or bicyclic aryl or heteroaryl ring having 1-4 heteroatoms independently selected from nitrogen, oxygen, and sulfur, or a 7-11 membered bicyclic saturated heterocyclic ring having 1-4 heteroatoms independently selected from nitrogen, oxygen, and sulfur. In some embodiments, each R^{A} is independently an optionally substituted group selected from C₁₋₆ aliphatic, a 3-7 membered saturated or partially unsaturated carbocyclic or heterocyclic ring having 1-2 heteroatoms independently selected from nitrogen, oxygen, and sulfur, or a 7-11 membered bicyclic heterocyclic ring having 1-4 heteroatoms independently selected from nitrogen, oxygen, and sulfur. In some embodiments, each R^{A} is independently an optionally substituted group selected from C₁₋₆ aliphatic, phenyl, naphthalenyl, a 3-7 membered saturated or partially unsaturated carbocyclic or heterocyclic ring having 1-2 heteroatoms independently selected from nitrogen, oxygen, and sulfur, or a 5-10 membered monocyclic or bicyclic aryl or heteroaryl ring having 1-4 heteroatoms independently selected from nitrogen, oxygen, and sulfur. In some embodiments, each R^{A} is independently an optionally substituted group selected from C₁₋₆ aliphatic, phenyl, naphthalenyl, a 3-7 membered saturated or partially unsaturated carbocyclic, or a 5-10 membered monocyclic or bicyclic aryl or heteroaryl ring having 1-4 heteroatoms independently selected from nitrogen, oxygen, and sulfur. In some embodiments, each R^{A} is independently an optionally substituted group selected from C₁₋₆ aliphatic, a 3-7 membered saturated or partially unsaturated carbocyclic or heterocyclic ring having 1-2 heteroatoms independently selected from nitrogen, oxygen, and sulfur, a 5-10 membered monocyclic or bicyclic heteroaryl ring having 1-4 heteroatoms independently selected from nitrogen, oxygen, and sulfur, or a 7-11 membered bicyclic heterocyclic ring having 1-4 heteroatoms independently selected from nitrogen, oxygen, and sulfur. In some embodiments, each R^{A} is independently an optionally substituted group selected from C₁₋₆ aliphatic, phenyl, naphthalenyl, a 3-7 membered saturated or partially unsaturated carbocyclic or heterocyclic ring having 1-2 heteroatoms independently selected from nitrogen, oxygen, and sulfur, a 5-6 membered monocyclic heteroaryl ring having 1-4 heteroatoms independently selected from nitrogen, oxygen, and sulfur, or a 7-11 membered bicyclic heterocyclic ring having 1-4 heteroatoms independently selected from nitrogen, oxygen, and sulfur. In some embodiments, each R^{A} is independently an optionally substituted group selected from C₁₋₆ aliphatic, phenyl, naphthalenyl, a 3-7 membered saturated or partially unsaturated carbocyclic or heterocyclic ring having 1-2 heteroatoms independently selected from nitrogen, oxygen, and sulfur, a 5-10 membered monocyclic or bicyclic aryl or a monocyclic heteroaryl ring having 1-4 heteroatoms independently selected from nitrogen, oxygen, and sulfur. In some embodiments, each R^{A} is independently an optionally substituted group selected from C₁₋₆ aliphatic, phenyl, naphthalenyl, a 3-7 membered saturated or partially unsaturated carbocyclic or heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each R^{A} is independently an optionally substituted group selected from C₁₋₆ aliphatic, phenyl, naphthalenyl, a 3-5 or 7 membered saturated or partially unsaturated carbocyclic or heterocyclic ring having 1-2 heteroatoms independently selected from nitrogen, oxygen, and sulfur, a 5-10 membered monocyclic or bicyclic aryl or heteroaryl ring having 1-4 heteroatoms independently selected from nitrogen, oxygen, and sulfur, or a 7-11 membered bicyclic heterocyclic ring having 1-4 heteroatoms independently selected from nitrogen, oxygen, and sulfur.

In some embodiments, R^{A} is an optionally substituted C₁₋₆ aliphatic. In some embodiments, R^{A} is an optionally substituted phenyl. In some embodiments, R^{A} is an optionally substituted 3-7 membered saturated or partially unsaturated carbocyclic. In some embodiments, R^{A} is an optionally substituted saturated or partially unsaturated heterocyclic ring having 1-2 heteroatoms independently selected from nitrogen, oxygen, and sulfur. In some embodiments. R^{A} is an optionally substituted R^{A} is a 5-10 membered monocyclic or bicyclic heteroaryl ring having 1-4 heteroatoms independently selected from nitrogen, oxygen, and sulfur. In some embodiments, R^{A} is an optionally substituted 5-6 membered heteroaryl ring having 1-4 heteroatoms independently selected from nitrogen, oxygen, and sulfur.

In some embodiments, R^{A} is optionally substituted naphthalenyl. In some embodiments, R^{A} is optionally substituted dihydrobenzo[b][1,4]dioxinyl.

In some embodiments, R^{A} is an optionally substituted 8-11 membered monocyclic or bicyclic aryl or heteroaryl with 1-4 heteroatoms independently selected from nitrogen, oxygen and sulfur. In some embodiments, R^{A} is optionally substituted naphthalenyl or dihydrobenzo[b][1,4]dioxinyl. In some embodiments, R^{A} is optionally substituted quinolinyl.

In some embodiments, R^{A} is C₁₋₆ alkyl (e.g., methyl, ethyl, isopropyl). In some embodiments, R^{A} is C₁₋₆ haloalkyl (e.g., —CF₃, —CHF₂).

In some embodiment, R^{A} is as depicted in the compounds of Table 1, below.

As described above and defined herein, each R^{B′} is independently -L^{B}-Cy^{B1}-H or -L^{B}-Cy^{B1}-Cy^{B2}.

In some embodiments, R^{B′} is -L^{B}-Cy^{B1}-H. In some embodiments, R^{B′} is -L^{B}-Cy^{B1}-Cy^{B2}.

In some embodiments, R^{B′} is

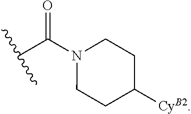

In some embodiments, R^{B′} is

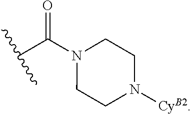

In some embodiments, R^{B′} is

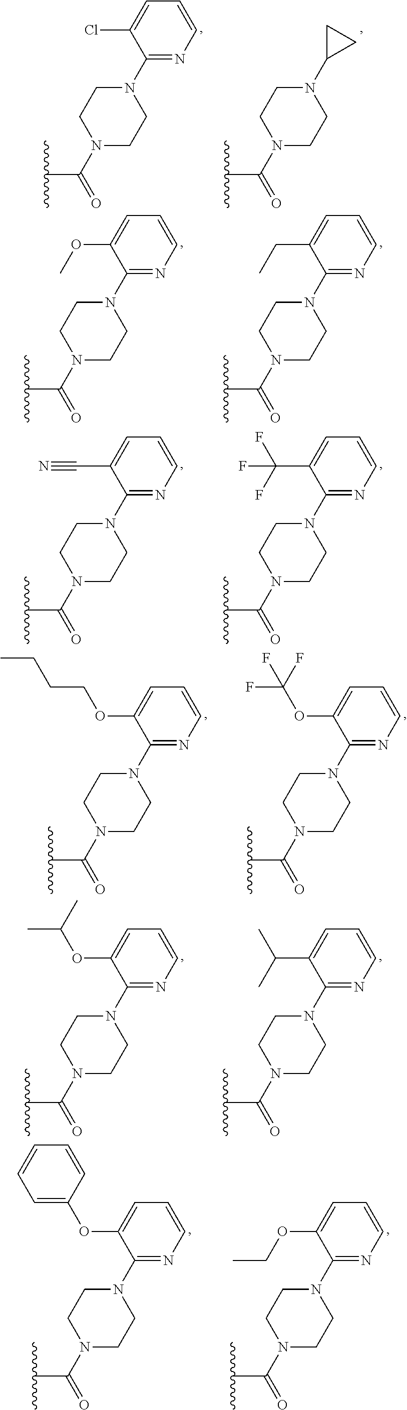

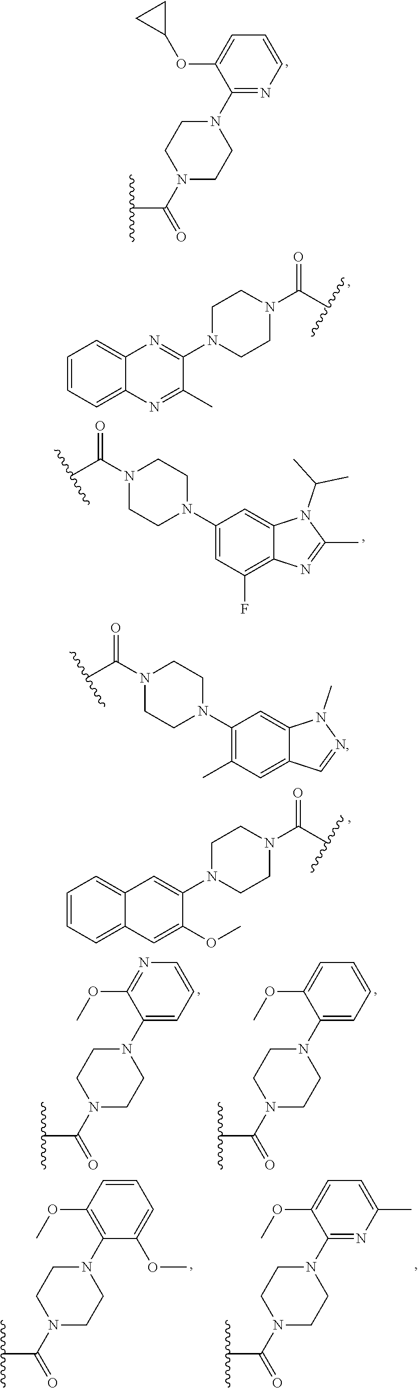

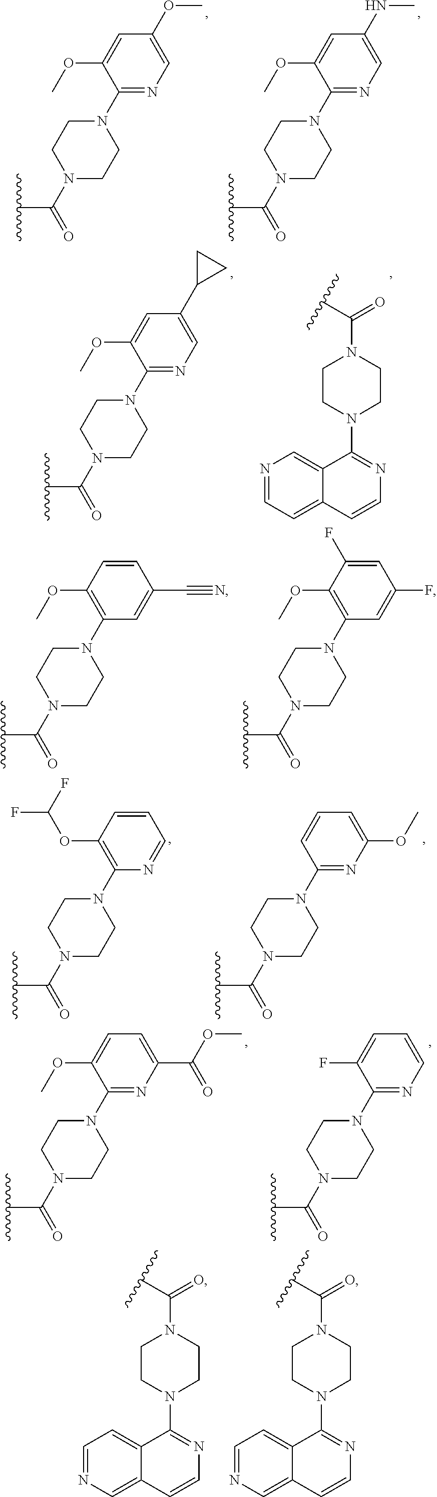

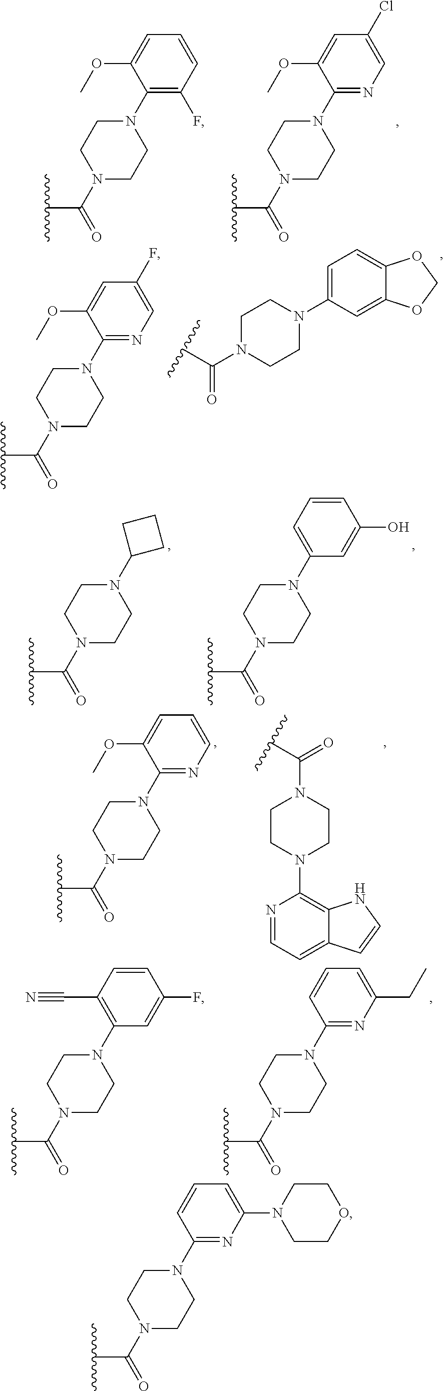

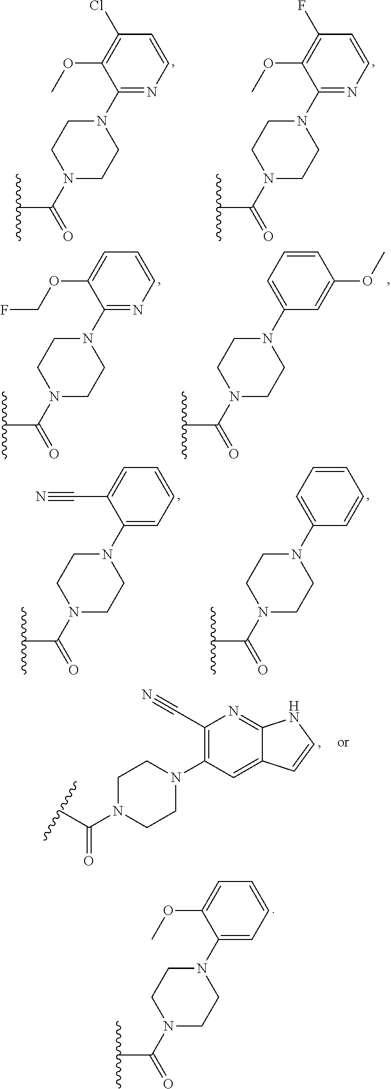

As described above and defined herein, each L^{B} is independently a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(NR)R—, —CR₂—, —CF₂—, —CRF—, —CR(OR)—, —NR—, —S—, —S(O)—, —S(O)₂— —S(O)(NR)— or —CR═CR—.

In some embodiments, each L^{B} is independently a covalent bond or a C₁₋₃ bivalent straight or branched saturated or unsaturated hydrocarbon chain wherein 1-2 methylene units of the chain are independently and optionally replaced with —O—, —C(O)—, —C(S)—, —CR₂—, —CF₂—, —CRF—, —CR(OR)—, —NR—, —S—, —S(O)—, —S(O)₂— —S(O)(NR)— or —CR═CR—.

In some embodiments, L^{B} is a covalent bond. In some embodiments, L^{B} is a C₁₋₃ bivalent straight or branched saturated or unsaturated hydrocarbon chain wherein 1-2 methylene units of the chain are independently and optionally replaced with —O—, —C(O)—, —C(S)—, —CR₂—, —CF₂—, —CRF—, —CR(OR)—, —NR—, —S—, —S(O)—, —S(O)₂—, —S(O)(NR)— or —CR═CR—. In some embodiments, L^{B} is a C₁₋₃ bivalent straight or branched saturated or unsaturated hydrocarbon chain wherein 1-2 methylene units of the chain are independently and optionally replaced with —O—, —C(O)—, —NR—, —S—, —S(O)—, or —S(O)₂—. In some embodiments, L^{B} is a C₁-3 bivalent straight or branched saturated or unsaturated hydrocarbon chain wherein 1-2 methylene units of the chain are independently and optionally replaced with —O—, —C(O)—, or —NR—. In some embodiments, L^{B} is a C₁₋₃ bivalent straight or branched saturated or unsaturated hydrocarbon chain wherein 1 methylene unit of the chain is optionally replaced with —O—, —C(O)—, —NR—, —S—, —S(O)—, or —S(O)₂—. In some embodiments, L^{B} is a C₁₋₃ bivalent straight or branched saturated or unsaturated hydrocarbon chain wherein 1 methylene unit of the chain is optionally replaced with —O—, —C(O)—, or —NR—. In some embodiments, L^{B} is a C₁₋₃ bivalent straight or branched saturated or unsaturated hydrocarbon chain wherein 1 methylene unit of the chain is replaced with —C(O)—. In some embodiments, L^{B} is —C(O)—.

In some embodiments, L^{B} is a C₁₋₃ bivalent straight or branched saturated or unsaturated hydrocarbon chain. In some embodiments, L^{B} is —CH₂—.

In some embodiments, L^{B} is —C(O)—, —C(S)—, —C(NR)R—, —S(O)—, —S(O)₂— or —S(O)(NR)—. In some embodiments, L^{B} is —C(O)—, —C(S)—, or —C(NR)R—. In some embodiments, L^{B} is —C(O)—, —S(O)—, —S(O)₂— or —S(O)(NR)—. In some embodiments, L^{B} is —C(S)—. In some embodiments, L^{B} is —C(NR)R—. In some embodiments, L^{B} is —S(O)—. In some embodiments, L^{B} is —S(O)₂—. In some embodiments, L^{B} is —S(O)(NR)—.

As described above and defined herein, each Cy^{B1} is independently an optionally substituted ring selected from phenylenyl, a 3-10 membered saturated or partially unsaturated monocyclic or bicyclic carbocyclylenyl or heterocyclylenyl having 1-4 heteroatoms independently selected from nitrogen, oxygen, and sulfur, and a 5-10 membered monocyclic or bicyclic arylenyl or heteroarylenyl having 1-4 heteroatoms independently selected from nitrogen, oxygen, and sulfur.

It will be appreciated that all embodiments to Cy^{B1} may refer to a terminal ring (or otherwise optionally substituted ring) in structures with -L^{B}-Cy^{B1}, or a ring further connected to Cy^{B2} as in structures -L^{B}-Cy^{B1}-Cy^{B2}, regardless of how presented. By way of example, an embodiment to Cy^{B1} is phenylenyl, refers to phenylenyl in -L^{B}-Cy^{B1}-Cy^{B2}, and phenyl in -L^{B}-Cy^{B1}. Similarly, an embodiment to Cy^{B1} is

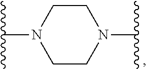 [SMILES: *N1CCN(*)CC1]

refers to

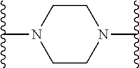 [SMILES: *N1CCN(*)CC1]

in -L^{B}-Cy^{B1}-Cy^{B2}, and

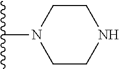 [SMILES: *N1CCNCC1]

in -L^{B}-Cy^{B1}. Similarly, an embodiment to Cy^{B1} is

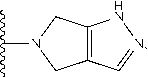 [SMILES: *N1Cc2cn[nH]c2C1]

refers to

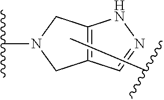

in -L^{B}Cy^{B1}-Cy^{B2} and

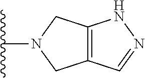 [SMILES: *N1Cc2cn[nH]c2C1]

in -L^{B}-Cy^{B1}.

In some embodiments, each Cy^{B1} is independently an optionally substituted ring selected from phenylenyl, a 3-7 membered saturated or partially unsaturated carbocyclylenyl or heterocyclylenyl having 1-4 heteroatoms independently selected from nitrogen, oxygen, and sulfur, and a 5-10 membered monocyclic or bicyclic arylenyl or heteroarylenyl having 1-4 heteroatoms independently selected from nitrogen, oxygen, and sulfur.

In some embodiments, Cy^{B1} is optionally substituted phenylenyl. In some embodiments, Cy^{B1} is phenylenyl.

In some embodiments, Cy^{B1} is optionally substituted 3-7 membered saturated or partially unsaturated carbocyclylenyl or heterocyclylenyl having 1-4 heteroatoms independently selected from nitrogen, oxygen, and sulfur. In some embodiments, Cy^{B1} is optionally substituted 5-10 membered monocyclic or bicyclic arylenyl or heteroarylenyl having 1-4 heteroatoms independently selected from nitrogen, oxygen, and sulfur.

In some embodiments, Cy^{B1} is optionally substituted 3-7 membered saturated or partially unsaturated heterocyclylenyl having 1-4 heteroatoms independently selected from nitrogen, oxygen, and sulfur. In some embodiments, Cy^{B1} is optionally substituted 6-membered saturated or partially unsaturated heterocyclylenyl having 1-4 heteroatoms independently selected from nitrogen, oxygen, and sulfur. In some embodiments, Cy^{B1} is optionally substituted 6-membered saturated or partially unsaturated heterocyclylenyl having 1-2 heteroatoms independently selected from nitrogen, oxygen, and sulfur. In some embodiments, Cy^{B1} is optionally substituted 6-membered saturated or partially unsaturated heterocyclylenyl having 1-2 nitrogen heteroatoms. In some embodiments, Cy^{B1} is optionally substituted piperadinylenyl or piperazinylenyl.

In some embodiments, Cy^{B1} is a 3-7 membered saturated or partially unsaturated heterocyclylenyl having 1-4 heteroatoms independently selected from nitrogen, oxygen, and sulfur. In some embodiments, Cy^{B1} is a 6-membered saturated or partially unsaturated heterocyclylenyl having 1-4 heteroatoms independently selected from nitrogen, oxygen, and sulfur. In some embodiments, Cy^{B1} is a 6-membered saturated or partially unsaturated heterocyclylenyl having 1-2 heteroatoms independently selected from nitrogen, oxygen, and sulfur. In some embodiments, Cy^{B1} is a 6-membered saturated or partially unsaturated heterocyclylenyl having 1-2 nitrogen heteroatoms. In some embodiments, Cy^{B1} is a piperadinylenyl or piperazinylenyl. In some embodiments, Cy^{B1} is

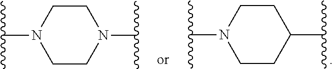 [SMILES: *N1CCN(*)CC1 ; *C1CCN(*)CC1]

In some embodiments, Cy^{B1} is an optionally substituted piperidinonyl or piperazinonyl. In some embodiments, Cy^{B1} is an optionally substituted dihydropyridinyl. In some embodiments, Cy^{B1} is an optionally substituted thiomorpholinyl.

In some embodiments, Cy^{B1} is a 3-10 membered saturated or partially unsaturated monocyclic or bicyclic heterocycylenyl having 1-4 heteroatoms independently selected from nitrogen, oxygen, and sulfur.

In some embodiments, Cy^{B1} is an optionally substituted 8-10 membered saturated or partially unsaturated bicyclic heterocyclylenyl having 1-4 heteroatoms independently selected from nitrogen, oxygen, and sulfur. In some embodiments, Cy^{B1} is an optionally substituted 8-membered saturated or partially unsaturated heterocyclylenyl having 1-4 heteroatoms independently selected from nitrogen, oxygen, and sulfur. In some embodiments, Cy^{B′} is an optionally substituted 8-membered saturated or partially unsaturated heterocyclylenyl having 1-2 heteroatoms independently selected from nitrogen, oxygen, and sulfur. In some embodiments, Cy^{B1} is an optionally substituted 8-membered saturated or partially unsaturated heterocyclylenyl having 1-2 nitrogen heteroatoms. In some embodiments, Cy^{B1} is optionally substituted

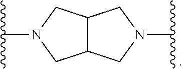 [SMILES: *N1CC2CN(*)CC2C1]

In some embodiments, Cy^{B1} is an optionally substituted

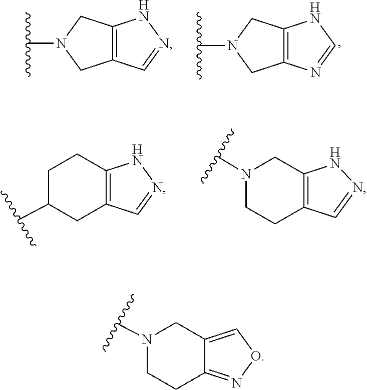

In some embodiments, Cy^{B1} is an optionally substituted 5-6 membered monocyclic heteroarylenyl having 1-4 heteroatoms independently selected from nitrogen, oxygen, and sulfur. In some embodiments, Cy^{B1} is an optionally substituted 6 membered monocyclic heteroarylenyl having 1-4 heteroatoms independently selected from nitrogen, oxygen, and sulfur. In some embodiments, Cy^{B1} is an optionally substituted pyridinonyl, pyrazinonyl, or pyrimidinoyl.

As described above and defined herein, each Cy^{B2} is independently an optionally substituted ring selected from phenyl, a 3-10 membered saturated or partially unsaturated monocyclic or bicyclic carbocyclic or heterocyclic ring having 1-4 heteroatoms independently selected from nitrogen, oxygen, and sulfur, and a 5-10 membered monocyclic or bicyclic aryl or heteroaryl with 1-4 heteroatoms independently selected from nitrogen, oxygen and sulfur.

As described above and defined herein, each Cy^{B2} is independently an optionally substituted ring selected from phenyl, a 3-10 membered saturated or partially unsaturated monocyclic or bicyclic carbocyclic or heterocyclic ring having 1-4 heteroatoms independently selected from nitrogen, oxygen, and sulfur, and a 5-10 membered monocyclic or bicyclic aryl or heteroaryl with 1-4 heteroatoms independently selected from nitrogen, oxygen and sulfur.

In some embodiments, each Cy^{B2} is independently an optionally ring selected from phenyl, a 3-7 membered saturated or partially unsaturated carbocyclic or heterocyclic ring having 1-2 heteroatoms independently selected from nitrogen, oxygen, and sulfur, and a 5-10 membered monocyclic or bicyclic aryl or heteroaryl with 1-4 heteroatoms independently selected from nitrogen, oxygen and sulfur.

In some embodiments, Cy^{B2} is optionally substituted phenyl. In some embodiments, Cy^{B2} is phenyl. In some embodiments. Cy^{B2} is phenyl, optionally substituted with —CN, halogen, —R^{∘}, or —OR^{∘}, wherein R^{∘} is hydrogen or C₁₋₆ aliphatic.

In some embodiments, Cy^{B2} is optionally substituted 3-7 membered saturated or partially unsaturated carbocyclic or heterocyclic ring having 1-2 heteroatoms independently selected from nitrogen, oxygen, and sulfur.

In some embodiments, Cy^{B2} is optionally substituted 3-7 membered saturated or partially unsaturated carbocyclic. In some embodiments, Cy^{B2} is optionally substituted cyclopropyl. In some embodiments, Cy^{B2} is cyclopropyl. In some embodiments. Cy^{B2} is optionally substituted cyclobutyl. In some embodiments, Cy^{B2} is cyclobutyl.

In some embodiments, Cy^{B2} is optionally substituted cyclopentyl. In some embodiments, Cy^{B2} is cyclopentyl. In some embodiments, Cy^{B2} is optionally substituted cyclohexyl. In some embodiments, Cy^{B2} is cyclohexyl.

In some embodiments, Cy^{B2} is optionally substituted 5-10 membered monocyclic or bicyclic aryl or heteroaryl with 1-4 heteroatoms independently selected from nitrogen, oxygen and sulfur.

In some embodiments, Cy^{B2} is optionally substituted 5-6 membered monocyclic heteroaryl with 1-4 heteroatoms independently selected from nitrogen, oxygen and sulfur. In some embodiments, Cy^{B2} is optionally substituted 5-6 membered monocyclic heteroaryl with 1-2 heteroatoms independently selected from nitrogen, oxygen and sulfur. In some embodiments. Cy^{B2} is optionally substituted pyridinyl. In some embodiments, Cy^{B2} is pyridinyl, optionally substituted with —CN, halogen, —R^{∘}, —OR^{∘}, —N(R^{∘})₂—C(O)OR^{∘}, wherein each R^{∘} is independently hydrogen; C₁₋₆ aliphatic, which may be optionally substituted with halogen, —(CH₂)₀₋₂OH, or —(CH₂)₀₋₂OR^{●}, where R^{●} is C₁₋₄ aliphatic; or a 5-6-membered saturated, partially unsaturated, or aryl ring having 0-4 heteroatoms independently selected from nitrogen, oxygen, or sulfur (e.g., phenyl or morpholinyl).

In some embodiments, Cy^{B2} is optionally substituted 6 membered monocyclic heteroaryl with 1-2 heteroatoms independently selected from nitrogen, oxygen and sulfur. In some embodiments, Cy^{B2} is optionally substituted pyridinyl, pyrimidinyl, pyridazinyl, pyrazinyl, or triazinyl.

In some embodiments. Cy^{B2} is optionally substituted optionally substituted pyridinonyl, pyrazinonyl, or pyrimidinoyl

In some embodiments, Cy^{B2} is optionally substituted 5 membered monocyclic heteroaryl with 1-2 heteroatoms independently selected from nitrogen, oxygen and sulfur. In some embodiments, Cy^{B2} is optionally substituted pyrazolyl, imidazolyl, or triazolyl.

In some embodiments, Cy^{B2} is optionally substituted 8-10 membered bicyclic aryl or heteroaryl with 1-4 heteroatoms independently selected from nitrogen, oxygen and sulfur.

In some embodiments, Cy^{B2} is optionally substituted 9-membered bicyclic heteroaryl with 1-4 heteroatoms independently selected from nitrogen, oxygen and sulfur. In some embodiments, Cy^{B2} is optionally substituted 9-membered bicyclic heteroaryl with 1-4 nitrogen heteroatoms. In some embodiments, Cy^{B2} is optionally substituted benzimidazolyl, indazolyl, or azaindolyl (e.g., pyrrolo[2,3-c]pyridinyl or pyrrolo[2,3-b]pyridinyl). In some embodiments, Cy^{B2} is benzimidazolyl indazolyl, or azaindolyl (e.g., pyrrolo[2,3-c]pyridinyl or pyrrolo[2,3-b]pyridinyl) optionally substituted with —CN, halogen or —R^{∘}, wherein R^{∘} is hydrogen or C₁₋₆ aliphatic. In some embodiments, Cy^{B2} is optionally substituted indolyl or azaindolyl.

In some embodiments, Cy^{B2} is optionally substituted 10-membered bicyclic aryl or heteroaryl with 1-4 heteroatoms independently selected from nitrogen, oxygen and sulfur. In some embodiments, Cy^{B2} is optionally substituted 10-membered bicyclic heteroaryl with 1-4 nitrogen heteroatoms. In some embodiments, Cy^{B2} is optionally substituted quinoxalinyl, isoquinolinyl, 2,6-naphthyridinyl, or 2,7-naphthyridinyl. In some embodiments. Cy^{B2} is quinoxalinyl, isoquinolinyl, 2,6-naphthyridinyl, or 2,7-naphthyridinyl, optionally substituted with —R^{∘}, wherein R^{∘} is hydrogen or C₁₋₆ aliphatic.

In some embodiments, Cy^{B2} is optionally substituted naphthalenyl. In some embodiments, Cy^{B2} is naphthalenyl, optionally substituted with —R^{∘} or —OR^{∘}, wherein R^{∘} is hydrogen or C₁₋₆ aliphatic.

In some embodiments, Cy^{B2} is optionally substituted benzo[d][1,3]dioxolyl.

In some embodiments, Cy^{B2} is

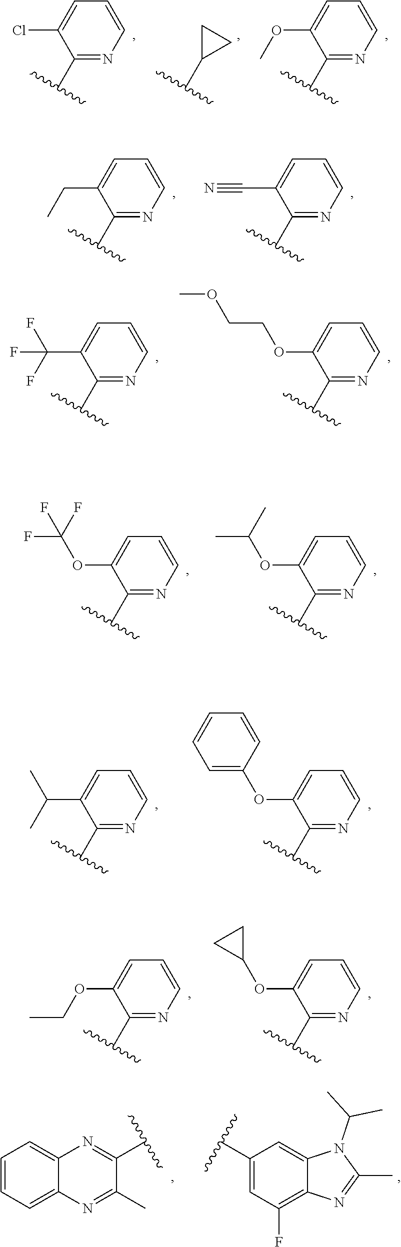

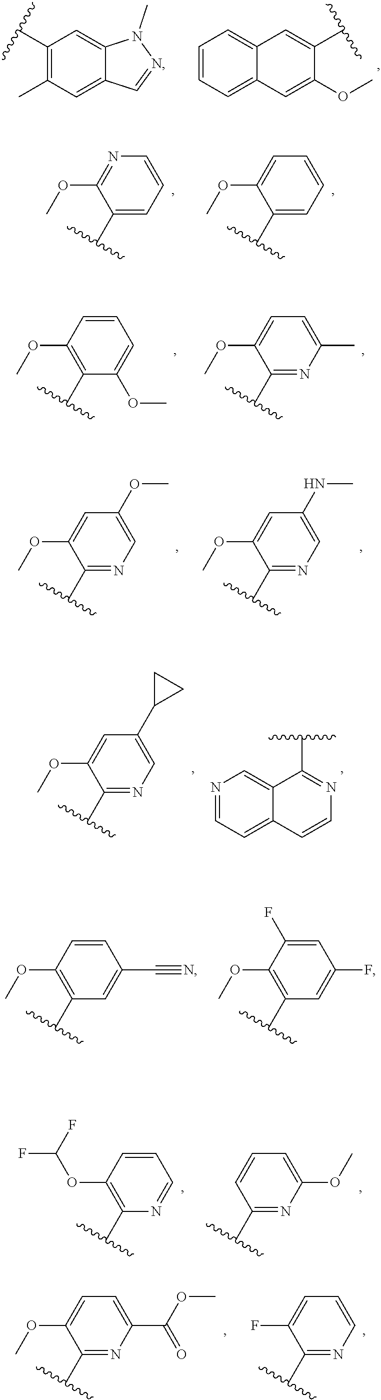

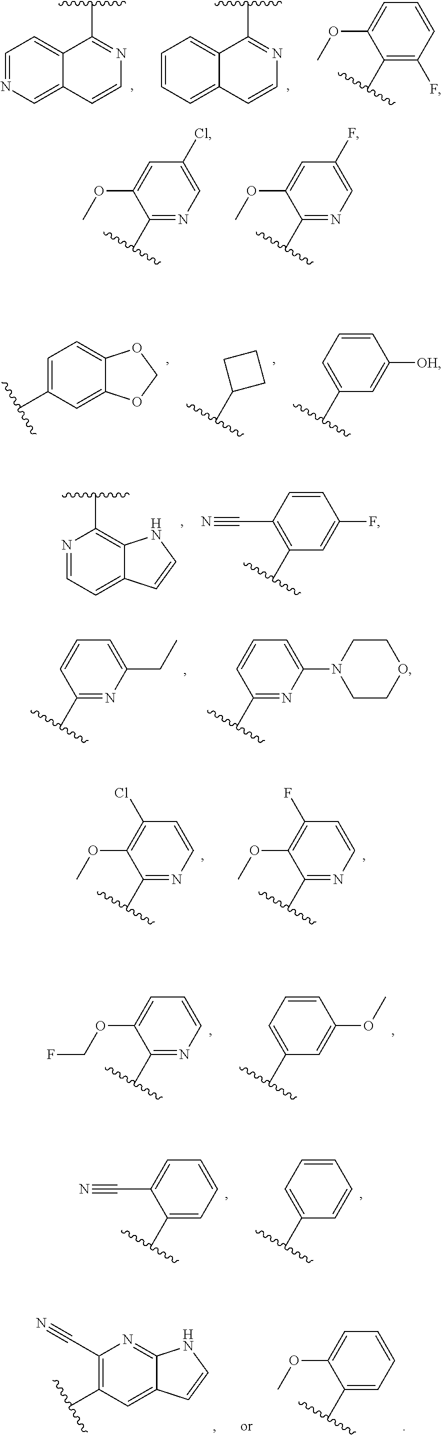

As described above and defined herein, each R is independently hydrogen, or an optionally substituted group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or two R groups on the same atom or adjacent atoms are optionally taken together with their intervening atoms to form a 3-10 membered saturated or partially unsaturated monocyclic, bicyclic, bridged bicyclic, or spirocyclic ring having 0-3 heteroatoms, in addition to the atom or adjacent atoms to which they are attached, independently selected from nitrogen, oxygen, and sulfur.

In some embodiments, R is hydrogen. In some embodiments, R is an optionally substituted C₁₋₆ aliphatic. In some embodiments, R is an optionally substituted phenyl. In some embodiments, R is an optionally substituted 4-7 membered saturated or partially unsaturated carbocyclic. In some embodiments, R is an optionally substituted 4-7 membered saturated or partially unsaturated heterocyclic ring having 1-2 heteroatoms independently selected from nitrogen, oxygen, and sulfur. In some embodiments, R is an optionally substituted 5-6 membered heteroaryl ring having 1-4 heteroatoms independently selected from nitrogen, oxygen, and sulfur. In some embodiments, two R groups on the same atom or adjacent atoms are optionally taken together with their intervening atoms to form a 3-10 membered saturated or partially unsaturated monocyclic, bicyclic, bridged bicyclic, or spirocyclic ring having 0-3 heteroatoms, in addition to the atom or adjacent atoms to which they are attached, independently selected from nitrogen, oxygen, and sulfur. In some embodiments, two R groups on adjacent atoms are optionally taken together with their intervening atoms to form a 3-7 membered saturated or partially unsaturated ring having 0-3 heteroatoms, in addition to the adjacent atoms to which they are attached, independently selected from nitrogen, oxygen, and sulfur.

In some embodiment, R is as depicted in the compounds of Table 1, below.

As described above and defined herein, U is a covalent bond or an optionally substituted C₁₋₅ bivalent straight or branched saturated or unsaturated hydrocarbon chain wherein 1-2 methylene units of the chain are independently and optionally replaced with —O—, —C(O)—, —C(S)—, —CR₂—, —NR—, —S—, —S(O)—, or —S(O)₂—.

In some embodiments, L^{x} is an optionally substituted C₁₋₄ bivalent straight or branched saturated or unsaturated hydrocarbon chain wherein 1-2 methylene units of the chain are independently and optionally replaced with —O—, —C(O)—, —C(S)—, —CR₂—, —NR—, —S—, —S(O)—, or —S(O)₂—. In some embodiments, L^{x} is an optionally substituted C₁₋₃ bivalent straight or branched saturated or unsaturated hydrocarbon chain wherein 1-2 methylene units of the chain are independently and optionally replaced with —O—, —C(O)—, —C(S)—, —CR₂—, —NR—, —S—, —S(O)—, or —S(O)₂—. In some embodiments, U is an optionally substituted C₃ bivalent straight or branched saturated or unsaturated hydrocarbon chain wherein 1-2 methylene units of the chain are independently and optionally replaced with —O—, —C(O)—, —C(S)—, —CR₂—, —NR—, —S—, —S(O)—, or —S(O)₂—.

In some embodiments, L^{x} is a covalent bond. In some embodiments, U is a C₁₋₅ bivalent straight or branched saturated or unsaturated hydrocarbon chain wherein 1-2 methylene units of the chain are independently and optionally replaced with —O—, —C(O)—, —C(S)—, —CR₂—, —NR—, —S—, —S(O)—, or —S(O)₂—. In some embodiments, L is a C₁₋₄ bivalent straight or branched saturated or unsaturated hydrocarbon chain wherein 1-2 methylene units of the chain are independently and optionally replaced with —O—, —C(O)—, —C(S)—, —CR₂—, —NR—, —S—, —S(O)—, or —S(O)₂—. In some embodiments, L^{x} is a C₁₋₃ bivalent straight or branched saturated or unsaturated hydrocarbon chain wherein 1-2 methylene units of the chain are independently and optionally replaced with —O—, —C(O)—, —C(S)—, —CR₂—, —NR—, —S—, —S(O)—, or —S(O)₂—. In some embodiments, L^{x} is a C₃ bivalent straight or branched saturated or unsaturated hydrocarbon chain wherein 1-2 methylene units of the chain are independently and optionally replaced with —O—, —C(O)—, —C(S)—, —CR₂—, —NR—, —S—, —S(O)—, or —S(O)₂—.

In some embodiments, L^{X} is an optionally substituted C₁₋₅ bivalent straight or branched saturated or unsaturated hydrocarbon chain wherein 1-2 methylene units of the chain are independently and optionally replaced with —O—, —C(O)—, —C(S)—, —CR₂—, —NR—, —S—, —S(O)—, or —S(O)₂—. In some embodiments, L^{X} is an optionally substituted C₁₋₅ bivalent straight or branched saturated or unsaturated hydrocarbon chain wherein 1-2 methylene units of the chain are independently and optionally replaced with —O—, —C(O)—, or —NR—.

In some embodiments, L^{X} is a C₁₋₅ bivalent straight or branched saturated or unsaturated hydrocarbon chain wherein 1-2 methylene units of the chain are independently and optionally replaced with —C(O)—, —C(S)—, —CR₂—, —S—, —S(O)—, or —S(O)₂, and wherein L^{X} is optionally substituted with halogen or —R^{∘}.

In some embodiments, L^{X} is an optionally substituted C₁₋₅ bivalent straight or branched saturated or unsaturated hydrocarbon chain wherein one methylene unit is replaced with —C(O)—. In some embodiments, L^{X} is an optionally substituted C₁₋₄ bivalent straight or branched saturated or unsaturated hydrocarbon chain wherein one methylene unit is replaced with —C(O)—. In some embodiments, L^{X} is an optionally substituted C₁₋₃ bivalent straight or branched saturated or unsaturated hydrocarbon chain wherein one methylene unit is replaced with —C(O)—. In some embodiments, L^{X} is an optionally substituted C₃ bivalent straight or branched saturated or unsaturated hydrocarbon chain wherein one methylene unit is replaced with —C(O)—. In some embodiments, L^{X} is an optionally substituted C₃ bivalent straight saturated or unsaturated hydrocarbon chain wherein one methylene unit is replaced with —C(O)—.

In some embodiments, L^{X} is a C₁₋₅ bivalent straight or branched saturated or unsaturated hydrocarbon chain wherein one methylene unit is replaced with —C(O)—, and wherein L^{X} is optionally substituted with halogen or —R^{∘}. In some embodiments, L^{X} is a C₁₋₄ bivalent straight or branched saturated or unsaturated hydrocarbon chain wherein one methylene unit is replaced with —C(O)—, and wherein L^{X} is optionally substituted with halogen or —R^{∘}. In some embodiments, L^{X} is a C₁₋₃ bivalent straight or branched saturated or unsaturated hydrocarbon chain wherein one methylene unit is replaced with —C(O)—, and wherein L^{X} is optionally substituted with halogen or —R^{∘}. In some embodiments, L^{X} is a C₃ bivalent straight or branched saturated or unsaturated hydrocarbon chain wherein one methylene unit is replaced with —C(O)— and wherein L^{X} is optionally substituted with halogen or —R^{∘}. In some embodiments, L^{X} is a C₃ bivalent straight saturated or unsaturated hydrocarbon chain wherein one methylene unit is replaced with —C(O)—, and wherein L^{X} is optionally substituted with halogen or —R^{∘}.

In some embodiments, L^{x} is —C(F)—CH-(optionally substituted C₁₋₃ bivalent straight or branched saturated or unsaturated hydrocarbon chain)-. In some embodiments, L^{x} is —C(CF₃)—N(R^{†})-(optionally substituted C₁₋₃ bivalent straight or branched saturated or unsaturated hydrocarbon chain)-

In some embodiments, L^{X} is a covalent bond or #-L^{XA}-L^{XB}-, wherein:
^{#} represents the point of attachment to Ring X:
L^{XA} is —C(O)—, —C(S)—, —CR₂—, —S(O)—, or —S(O)₂—; and
L^{XB} is a covalent bond or an optionally substituted C₁₋₄ bivalent straight or branched saturated or unsaturated hydrocarbon chain wherein 1-2 methylene units of the chain are independently and optionally replaced with —O—, —C(O)—, —C(S)—, —CR₂—, —NR—, —S—, —S(O)—, or —S(O)₂—.

As described above and defined herein, L^{XA} is —C(O)—, —C(S)—, —CR₂—, —C(NR)—, —S(O)—, or —S(O)₂—. In some embodiments, L^{XA} is —C(O)—, —C(S)—, —CR₂—, —S(O)—, —S(O)₂— or —S(O)(NR)—.

In some embodiments, L^{XA} is —C(O)— or —C(S)—. In some embodiments. L^{XA} is —C(O)—, —S(O)—, or —S(O)₂—. In some embodiments, L^{XA} is —S(O)— or —S(O)₂—. In some embodiments, L^{XA} is —C(O)—. In some embodiments, L^{XA} is —C(S)—. In some embodiments, L^{XA} is —CR₂—. In some embodiments, L^{XA} is —CR₂—, wherein each R of L^{XA} is independently hydrogen or optionally substituted C₁₋₆ aliphatic. In some embodiments, L^{XA} is —CR₂—, wherein each R of L^{XA} is independently hydrogen or C₁₋₆ aliphatic substituted with halogen (e.g., fluoro). In some embodiments, L^{XA} is —S(O)—. In some embodiments, L^{XA} is —S(O)₂—.

As described above and defined herein, L^{XB} is a covalent bond or an optionally substituted C₁₋₄ bivalent straight or branched saturated or unsaturated hydrocarbon chain wherein 1-2 methylene units of the chain are independently and optionally replaced with —O—, —C(O)—, —C(S)—, —CR₂—, —NR—, —S—, —S(O)—, or —S(O)₂—.

In some embodiments, L^{XB} is a covalent bond. In some embodiments, L^{XB} is an optionally substituted C₁₋₄ bivalent straight or branched saturated or unsaturated hydrocarbon chain wherein 1-2 methylene units of the chain are independently and optionally replaced with —O—, —C(O)—, —C(S)—, —CR₂—, —NR—, —S—, —S(O)—, or —S(O)₂—. In some embodiments, L^{XB} an optionally substituted C₁₋₄ bivalent straight or branched saturated or unsaturated hydrocarbon chain. In some embodiments, L^{XB} is an optionally substituted C₁₋₄ bivalent straight or branched saturated or unsaturated hydrocarbon chain, wherein 1 methylene unit of the chain is optionally replaced with —O—, —NR—, or —S—.

In some embodiments, L^{XB} is an optionally substituted C₁₋₃ bivalent straight or branched saturated or unsaturated hydrocarbon chain. In some embodiments, L^{XB} is an optionally substituted C₁₋₃ bivalent straight or branched saturated or unsaturated hydrocarbon chain, wherein 1 methylene unit of the chain is optionally replaced with —O—, —NR—, or —S—. In some embodiments, L^{XB} is —NR-(optionally substituted C₁₋₄ bivalent straight or branched saturated or unsaturated hydrocarbon chain)-. In some embodiments. L^{XB} is —O-(optionally substituted C₁₋₄ bivalent straight or branched saturated or unsaturated hydrocarbon chain)-. In some embodiments, L^{XB} is —S-(optionally substituted C₁₋₃ bivalent straight or branched saturated or unsaturated hydrocarbon chain)-.

In some embodiments, L^{XB} is optionally substituted —CH₂—, —CH₂CH₂—, —CH₂CH₂CH₂—, —CH₂CH₂CH₂O—, —CH₂C(O)O—, —CH₂CH₂C(O)O—, —NR—, or —NRCH₂CH₂—. In some embodiments, L^{XB} is —CH₂—, —CH₂CH₂—, —CH₂CH₂CH₂—, —CH₂CH₂CH₂O—, —CH₂C(O)O—, —CH₂CH₂C(O)O—, O

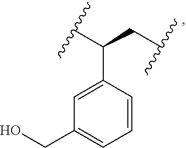 [SMILES: *C[C@H](*)c1cccc(CO)c1]

—N(CH₃)—, —CH(CH₃)—,

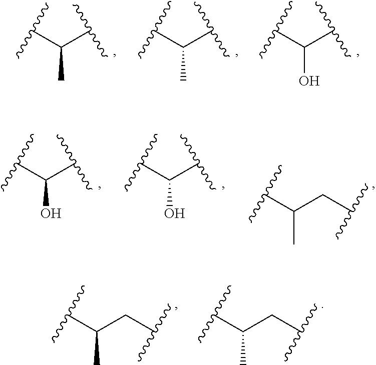

In some embodiments, L^{x} is —C(O)(CR₂)₁₋₃—. In some embodiments, L^{x} is —C(O)(CH₂)₁₋₃—.

In some embodiments, L^{x} is —C(O)—, —C(O)CH₂—, —S(O)₂CH₂—, —C(O)CH₂CH₂—, —C(O)OCH₂—, —C(O)CH₂O—, —C(O)CH₂CH₂CH₂—, —C(O)CH₂CH₂S(O)₂—, —C(O)CH₂CH₂CO₂—, —C(O)CH₂NHC(O)—,

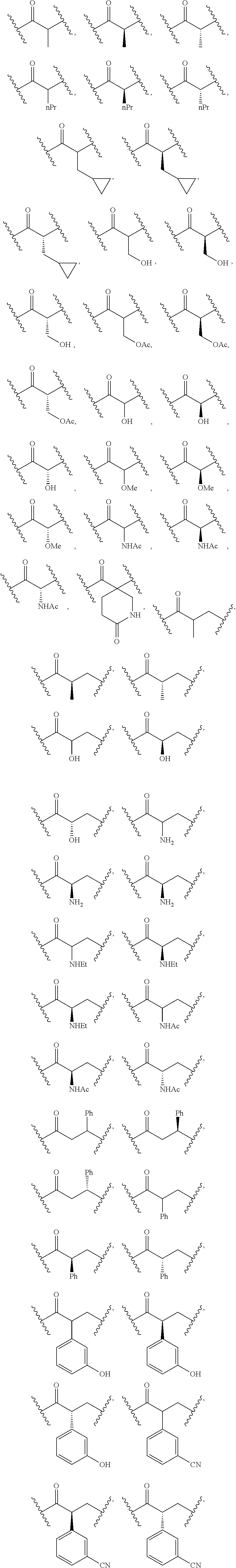

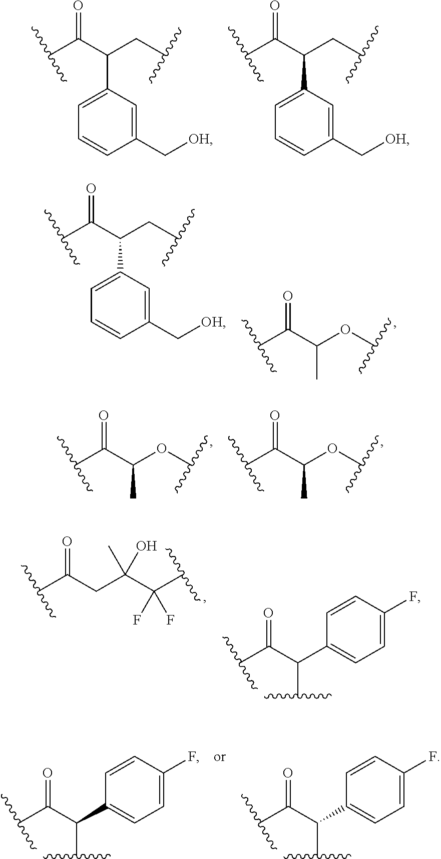

In some embodiments, L^{x} is —C(O)CH₂CH₂—.

In some embodiments, L^{x} is —S(O)₂CH₂CH₂—.

In some embodiment. L^{x} is as depicted in the compounds of Table 1, below.

In some embodiments, -Ring X-L^{x}- is:

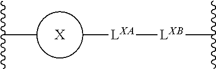

or a pharmaceutically acceptable salt thereof, wherein each of Ring X, L^{XA}, and L^{XB} is as defined above and described herein.

In some embodiments. —Ring X-L^{x}- is:

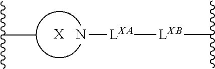

or a pharmaceutically acceptable salt thereof, wherein each of Ring X, L^{XA}, and L^{XB} is as defined above and described herein.

In some embodiments, —Ring X-L^{x}- is:

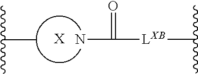

or a pharmaceutically acceptable salt thereof, wherein each of Ring X and L^{XB} is as defined above and described herein.

As described above and defined herein, each of w, x, and y are independently 0, 1, 2, 3, or 4.

In some embodiments, w is 0. In some embodiments, w is 1. In some embodiments, w is 2. In some embodiments, w is 3. In some embodiments, w is 4. In some embodiments, w is 0 or 1. In some embodiments, w is 1 or 2.

In some embodiments, x is 0. In some embodiments, x is 1. In some embodiments, x is 2. In some embodiments, x is 3. In some embodiments, x is 4. In some embodiments, x is 0 or 1. In some embodiments, x is 1 or 2.

In some embodiments, y is 0. In some embodiments, y is 1. In some embodiments, y is 2. In some embodiments, y is 3. In some embodiments, y is 4. In some embodiments, y is 0 or 1. In some embodiments, y is 1 or 2.

In some embodiment, w, x, and y are as depicted in the compounds of Table 1, below.

In some embodiments, SBM is

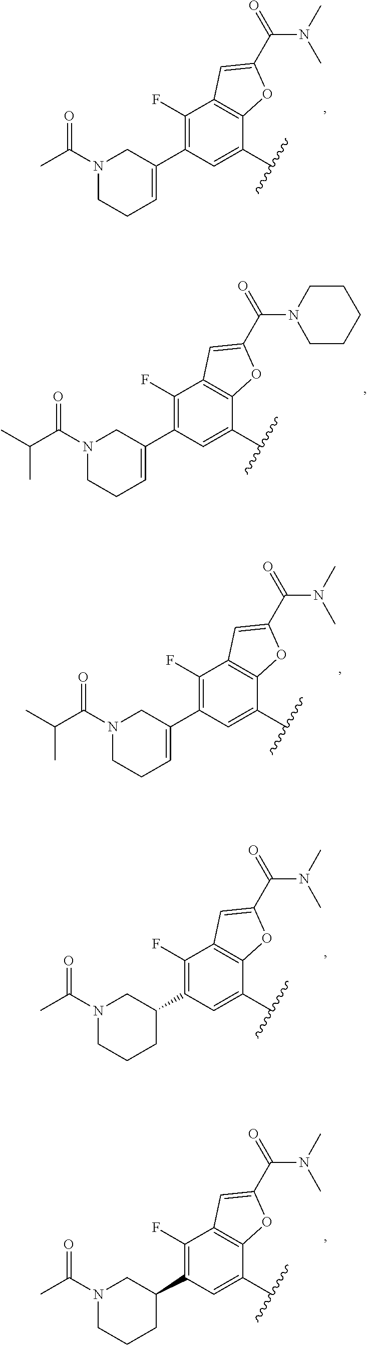

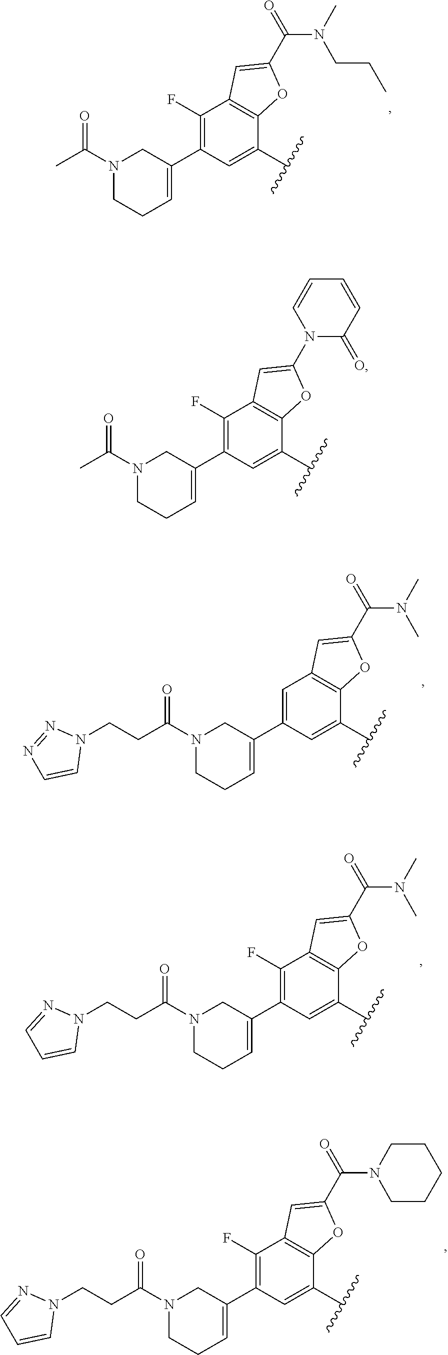

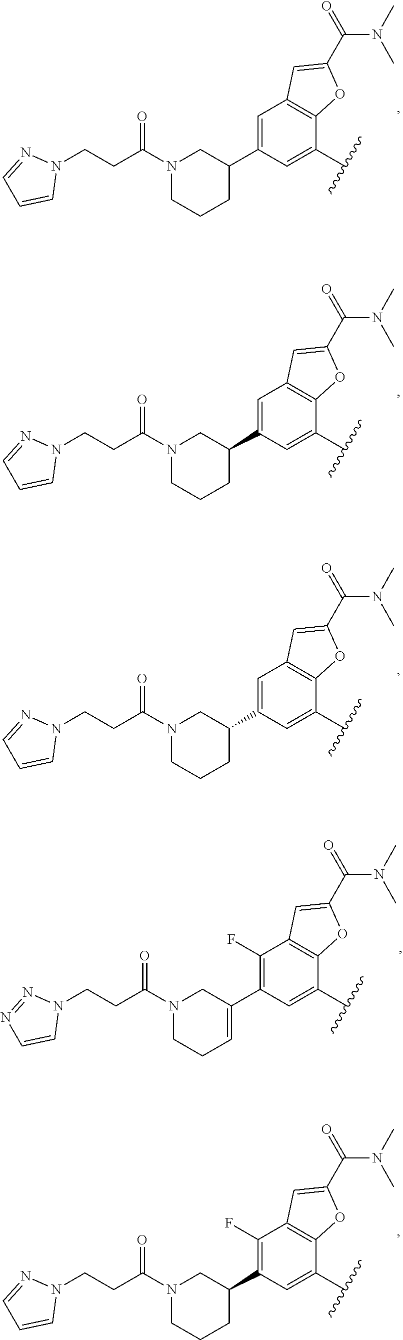 [SMILES: *c1cc(C2CCCN(C(=O)CCn3cccn3)C2)cc2cc(C(=O)N(C)C)oc12 ; *c1cc([C@H]2CCCN(C(=O)CCn3cccn3)C2)cc2cc(C(=O)N(C)C)oc12 ; *c1cc([C@@H]2CCCN(C(=O)CCn3cccn3)C2)cc2cc(C(=O)N(C)C)oc12 ; *c1cc(C2=CCCN(C(=O)CCn3ccnn3)C2)c(F)c2cc(C(=O)N(C)C)oc12 ; *c1cc([C@H]2CCCN(C(=O)CCn3cccn3)C2)c(F)c2cc(C(=O)N(C)C)oc12]

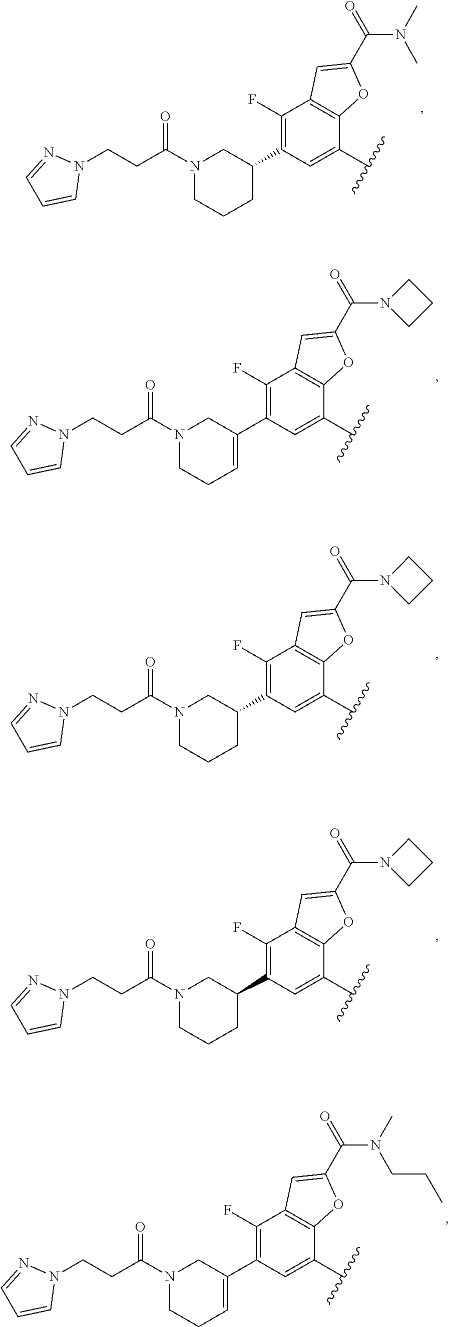 [SMILES: *c1cc([C@@H]2CCCN(C(=O)CCn3cccn3)C2)c(F)c2cc(C(=O)N(C)C)oc12 ; *c1cc(C2=CCCN(C(=O)CCn3cccn3)C2)c(F)c2cc(C(=O)N3CCC3)oc12 ; *c1cc([C@@H]2CCCN(C(=O)CCn3cccn3)C2)c(F)c2cc(C(=O)N3CCC3)oc12 ; *c1cc([C@H]2CCCN(C(=O)CCn3cccn3)C2)c(F)c2cc(C(=O)N3CCC3)oc12 ; *c1cc(C2=CCCN(C(=O)CCn3cccn3)C2)c(F)c2cc(C(=O)N(C)CCC)oc12]

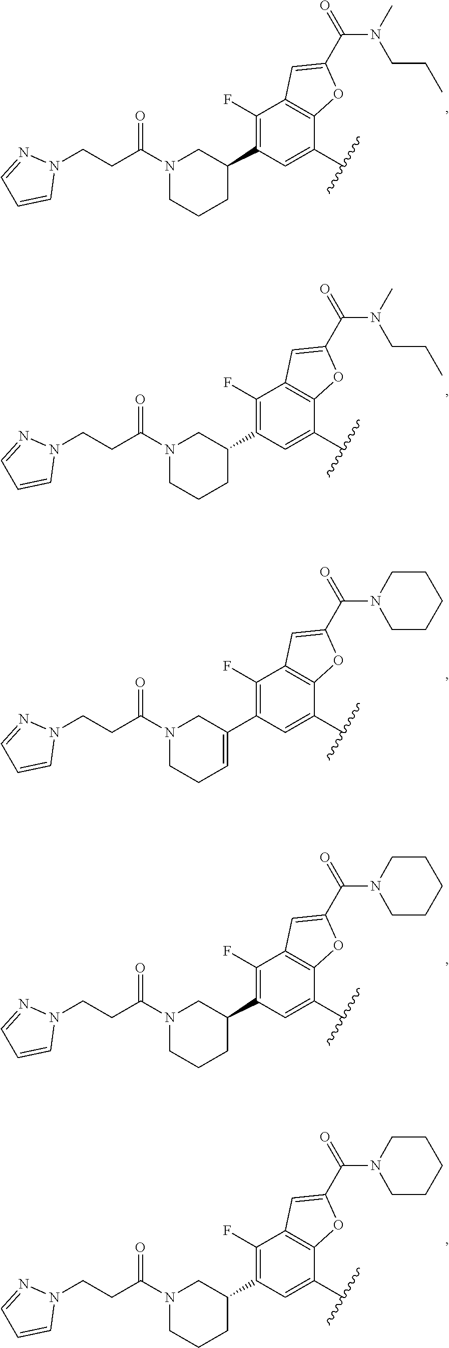 [SMILES: *c1cc([C@H]2CCCN(C(=O)CCn3cccn3)C2)c(F)c2cc(C(=O)N(C)CCC)oc12 ; *c1cc([C@@H]2CCCN(C(=O)CCn3cccn3)C2)c(F)c2cc(C(=O)N(C)CCC)oc12 ; *c1cc(C2=CCCN(C(=O)CCn3cccn3)C2)c(F)c2cc(C(=O)N3CCCCC3)oc12 ; *c1cc([C@H]2CCCN(C(=O)CCn3cccn3)C2)c(F)c2cc(C(=O)N3CCCCC3)oc12 ; *c1cc([C@@H]2CCCN(C(=O)CCn3cccn3)C2)c(F)c2cc(C(=O)N3CCCCC3)oc12]

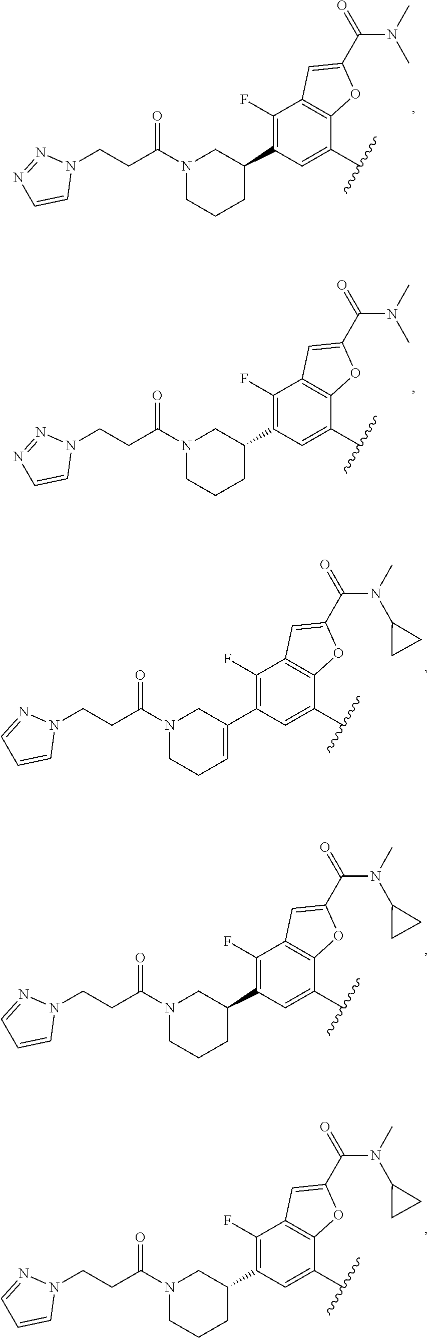 [SMILES: *c1cc([C@H]2CCCN(C(=O)CCn3ccnn3)C2)c(F)c2cc(C(=O)N(C)C)oc12 ; *c1cc([C@@H]2CCCN(C(=O)CCn3ccnn3)C2)c(F)c2cc(C(=O)N(C)C)oc12 ; *c1cc(C2=CCCN(C(=O)CCn3cccn3)C2)c(F)c2cc(C(=O)N(C)C3CC3)oc12 ; *c1cc([C@H]2CCCN(C(=O)CCn3cccn3)C2)c(F)c2cc(C(=O)N(C)C3CC3)oc12 ; *c1cc([C@@H]2CCCN(C(=O)CCn3cccn3)C2)c(F)c2cc(C(=O)N(C)C3CC3)oc12]

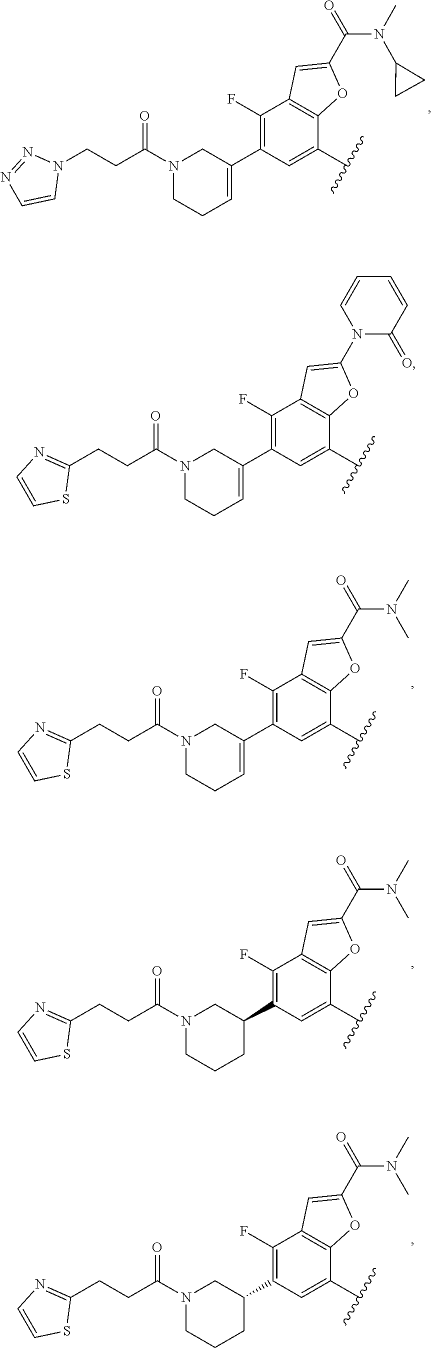 [SMILES: *c1cc(C2=CCCN(C(=O)CCn3ccnn3)C2)c(F)c2cc(C(=O)N(C)C3CC3)oc12 ; *c1cc(C2=CCCN(C(=O)CCc3nccs3)C2)c(F)c2cc(-n3ccccc3=O)oc12 ; *c1cc(C2=CCCN(C(=O)CCc3nccs3)C2)c(F)c2cc(C(=O)N(C)C)oc12 ; *c1cc([C@H]2CCCN(C(=O)CCc3nccs3)C2)c(F)c2cc(C(=O)N(C)C)oc12 ; *c1cc([C@@H]2CCCN(C(=O)CCc3nccs3)C2)c(F)c2cc(C(=O)N(C)C)oc12]

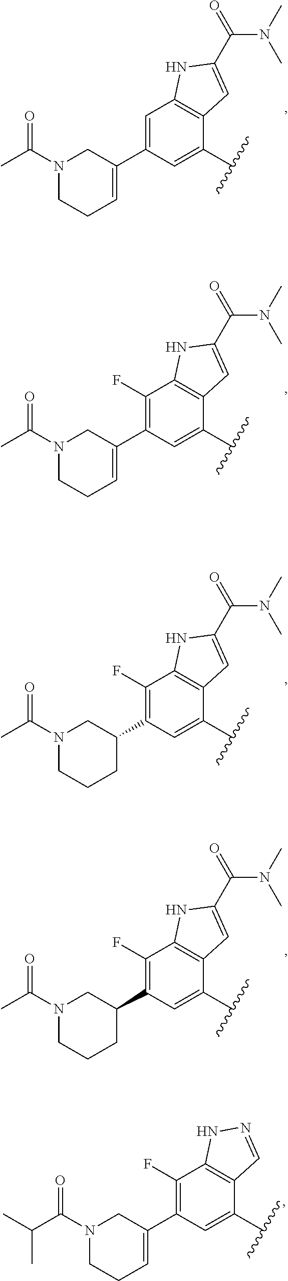

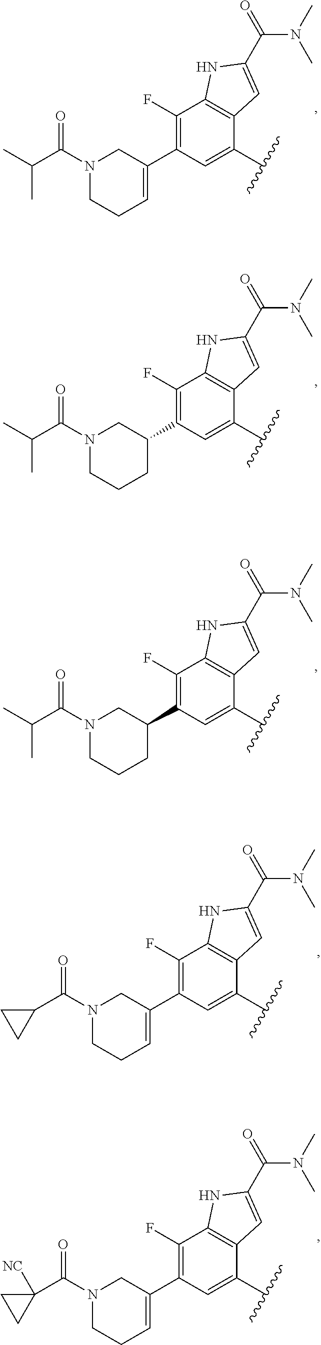

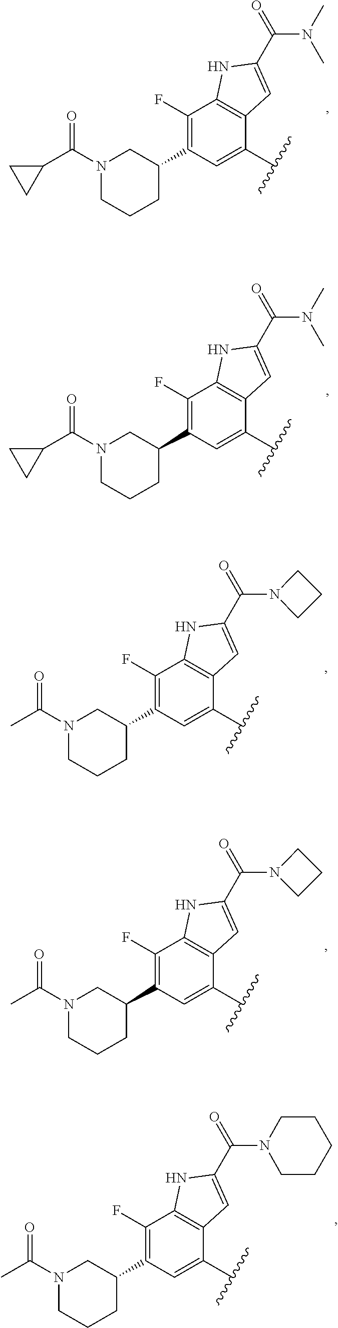

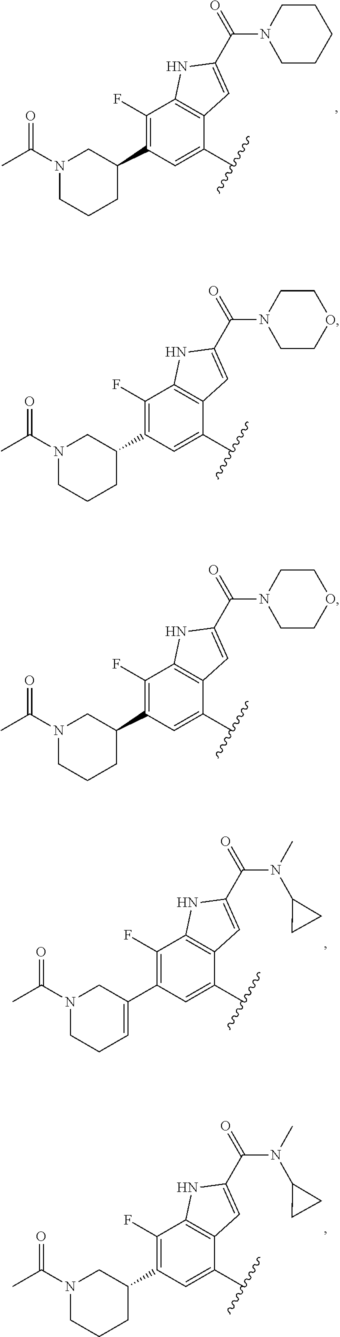 [SMILES: *c1cc([C@H]2CCCN(C(C)=O)C2)c(F)c2[nH]c(C(=O)N3CCCCC3)cc12 ; *c1cc([C@@H]2CCCN(C(C)=O)C2)c(F)c2[nH]c(C(=O)N3CCOCC3)cc12 ; *c1cc([C@H]2CCCN(C(C)=O)C2)c(F)c2[nH]c(C(=O)N3CCOCC3)cc12 ; *c1cc(C2=CCCN(C(C)=O)C2)c(F)c2[nH]c(C(=O)N(C)C3CC3)cc12 ; *c1cc([C@@H]2CCCN(C(C)=O)C2)c(F)c2[nH]c(C(=O)N(C)C3CC3)cc12]

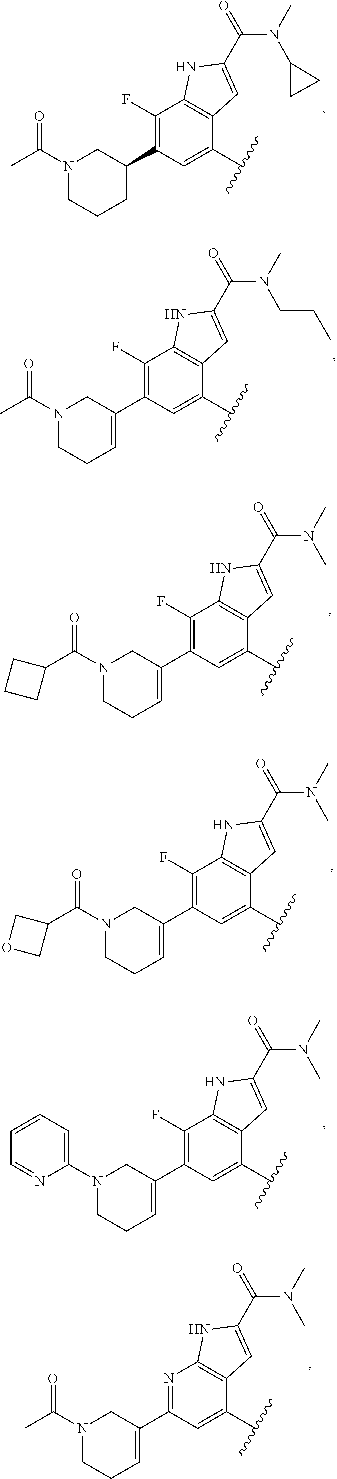

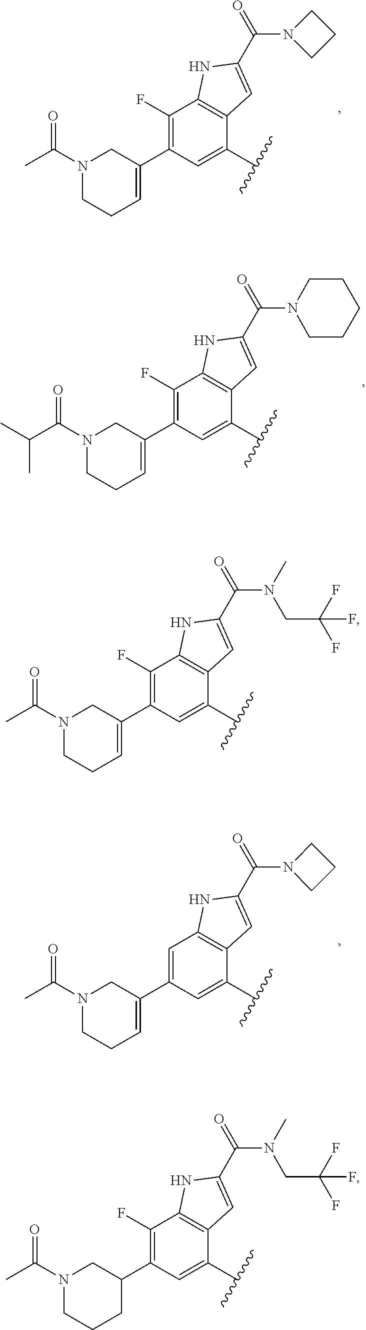 [SMILES: *c1cc(C2=CCCN(C(C)=O)C2)c(F)c2[nH]c(C(=O)N3CCC3)cc12 ; *c1cc(C2=CCCN(C(=O)C(C)C)C2)c(F)c2[nH]c(C(=O)N3CCCCC3)cc12 ; *c1cc(C2=CCCN(C(C)=O)C2)c(F)c2[nH]c(C(=O)N(C)CC(F)(F)F)cc12 ; *c1cc(C2=CCCN(C(C)=O)C2)cc2[nH]c(C(=O)N3CCC3)cc12 ; *c1cc(C2CCCN(C(C)=O)C2)c(F)c2[nH]c(C(=O)N(C)CC(F)(F)F)cc12]

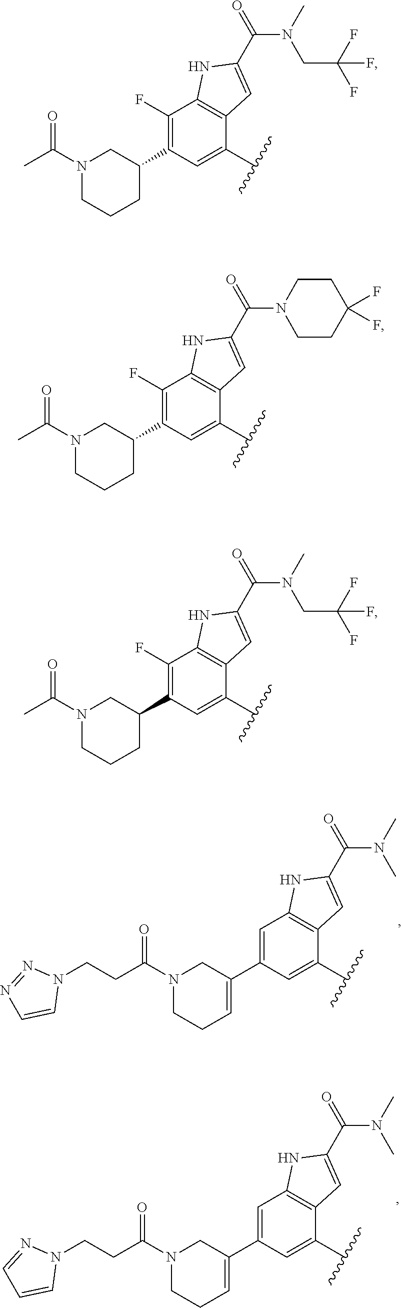 [SMILES: *c1cc([C@@H]2CCCN(C(C)=O)C2)c(F)c2[nH]c(C(=O)N(C)CC(F)(F)F)cc12 ; *c1cc([C@@H]2CCCN(C(C)=O)C2)c(F)c2[nH]c(C(=O)N3CCC(F)(F)CC3)cc12 ; *c1cc([C@H]2CCCN(C(C)=O)C2)c(F)c2[nH]c(C(=O)N(C)CC(F)(F)F)cc12 ; *c1cc(C2=CCCN(C(=O)CCn3ccnn3)C2)cc2[nH]c(C(=O)N(C)C)cc12 ; *c1cc(C2=CCCN(C(=O)CCn3cccn3)C2)cc2[nH]c(C(=O)N(C)C)cc12]

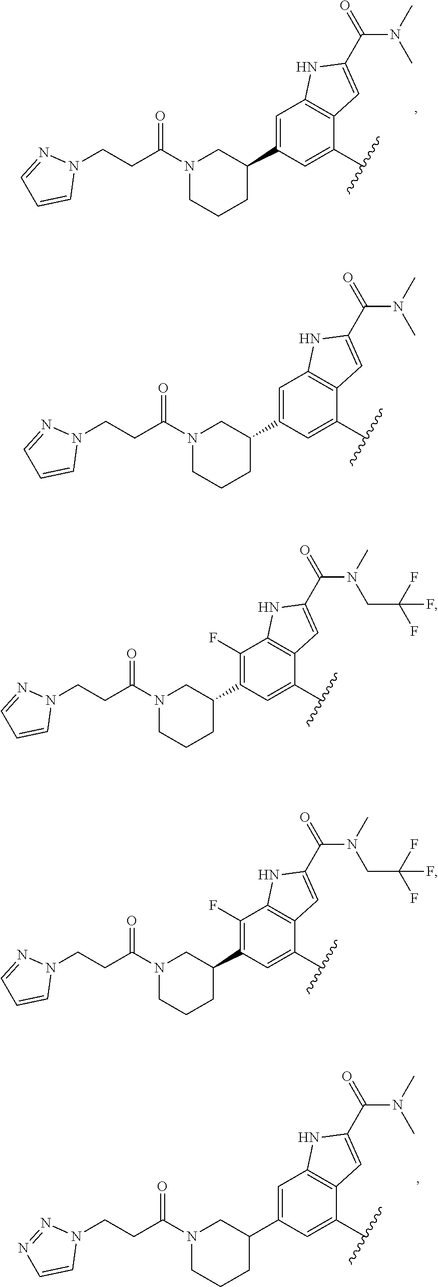 [SMILES: *c1cc([C@H]2CCCN(C(=O)CCn3cccn3)C2)cc2[nH]c(C(=O)N(C)C)cc12 ; *c1cc([C@@H]2CCCN(C(=O)CCn3cccn3)C2)cc2[nH]c(C(=O)N(C)C)cc12 ; *c1cc([C@@H]2CCCN(C(=O)CCn3cccn3)C2)c(F)c2[nH]c(C(=O)N(C)CC(F)(F)F)cc12 ; *c1cc([C@H]2CCCN(C(=O)CCn3cccn3)C2)c(F)c2[nH]c(C(=O)N(C)CC(F)(F)F)cc12 ; *c1cc(C2CCCN(C(=O)CCn3ccnn3)C2)cc2[nH]c(C(=O)N(C)C)cc12]

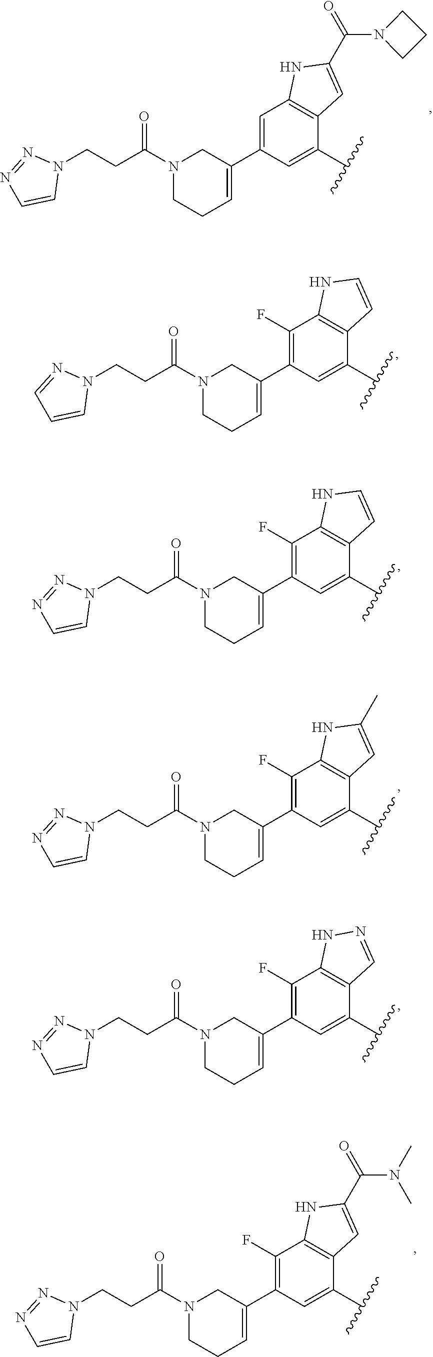 [SMILES: *c1cc(C2=CCCN(C(=O)CCn3ccnn3)C2)cc2[nH]c(C(=O)N3CCC3)cc12 ; *c1cc(C2=CCCN(C(=O)CCn3cccn3)C2)c(F)c2[nH]ccc12 ; *c1cc(C2=CCCN(C(=O)CCn3ccnn3)C2)c(F)c2[nH]ccc12 ; *c1cc(C2=CCCN(C(=O)CCn3ccnn3)C2)c(F)c2[nH]c(C)cc12 ; *c1cc(C2=CCCN(C(=O)CCn3ccnn3)C2)c(F)c2[nH]ncc12 ; *c1cc(C2=CCCN(C(=O)CCn3ccnn3)C2)c(F)c2[nH]c(C(=O)N(C)C)cc12]

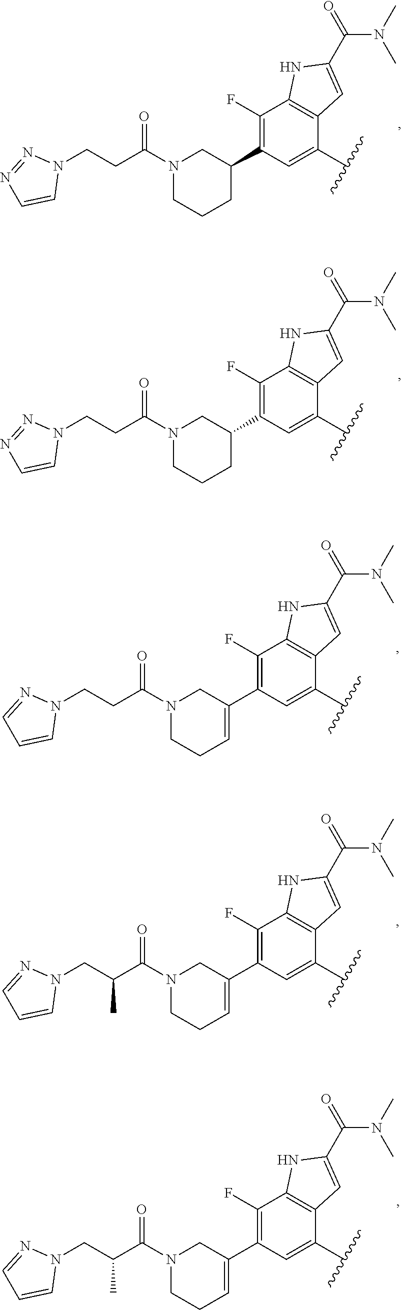 [SMILES: *c1cc([C@H]2CCCN(C(=O)CCn3ccnn3)C2)c(F)c2[nH]c(C(=O)N(C)C)cc12 ; *c1cc([C@@H]2CCCN(C(=O)CCn3ccnn3)C2)c(F)c2[nH]c(C(=O)N(C)C)cc12 ; *c1cc(C2=CCCN(C(=O)CCn3cccn3)C2)c(F)c2[nH]c(C(=O)N(C)C)cc12 ; *c1cc(C2=CCCN(C(=O)[C@@H](C)Cn3cccn3)C2)c(F)c2[nH]c(C(=O)N(C)C)cc12 ; *c1cc(C2=CCCN(C(=O)[C@H](C)Cn3cccn3)C2)c(F)c2[nH]c(C(=O)N(C)C)cc12]

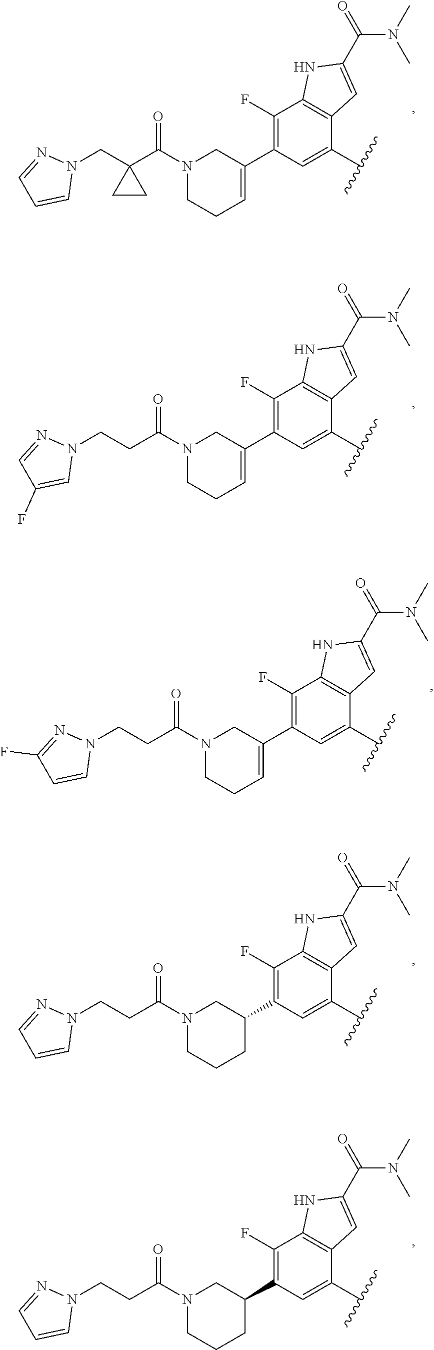 [SMILES: *c1cc(C2=CCCN(C(=O)C3(Cn4cccn4)CC3)C2)c(F)c2[nH]c(C(=O)N(C)C)cc12 ; *c1cc(C2=CCCN(C(=O)CCn3cc(F)cn3)C2)c(F)c2[nH]c(C(=O)N(C)C)cc12 ; *c1cc(C2=CCCN(C(=O)CCn3ccc(F)n3)C2)c(F)c2[nH]c(C(=O)N(C)C)cc12 ; *c1cc([C@@H]2CCCN(C(=O)CCn3cccn3)C2)c(F)c2[nH]c(C(=O)N(C)C)cc12 ; *c1cc([C@H]2CCCN(C(=O)CCn3cccn3)C2)c(F)c2[nH]c(C(=O)N(C)C)cc12]

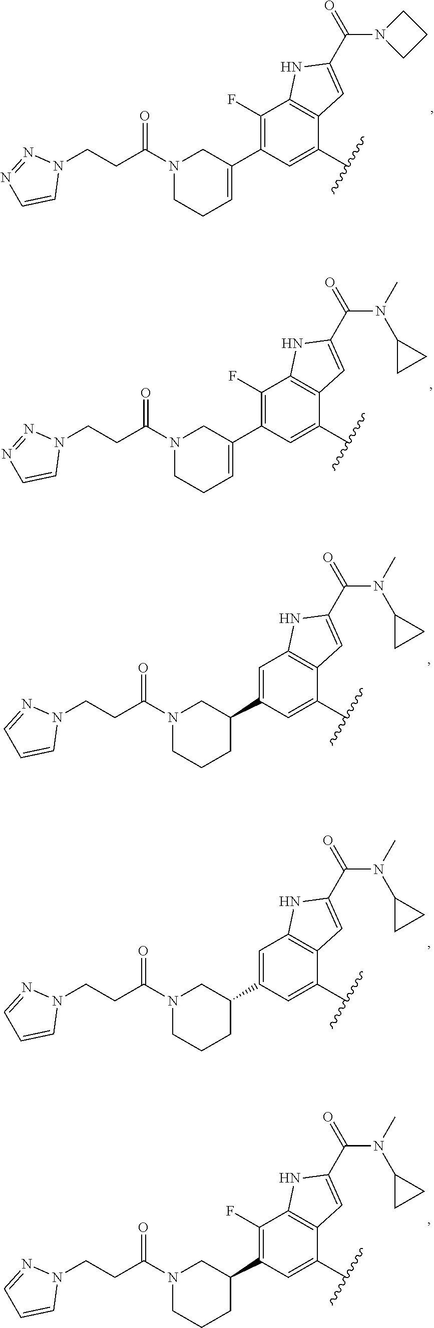 [SMILES: *c1cc(C2=CCCN(C(=O)CCn3ccnn3)C2)c(F)c2[nH]c(C(=O)N3CCC3)cc12 ; *c1cc(C2=CCCN(C(=O)CCn3ccnn3)C2)c(F)c2[nH]c(C(=O)N(C)C3CC3)cc12 ; *c1cc([C@H]2CCCN(C(=O)CCn3cccn3)C2)cc2[nH]c(C(=O)N(C)C3CC3)cc12 ; *c1cc([C@@H]2CCCN(C(=O)CCn3cccn3)C2)cc2[nH]c(C(=O)N(C)C3CC3)cc12 ; *c1cc([C@H]2CCCN(C(=O)CCn3cccn3)C2)c(F)c2[nH]c(C(=O)N(C)C3CC3)cc12]

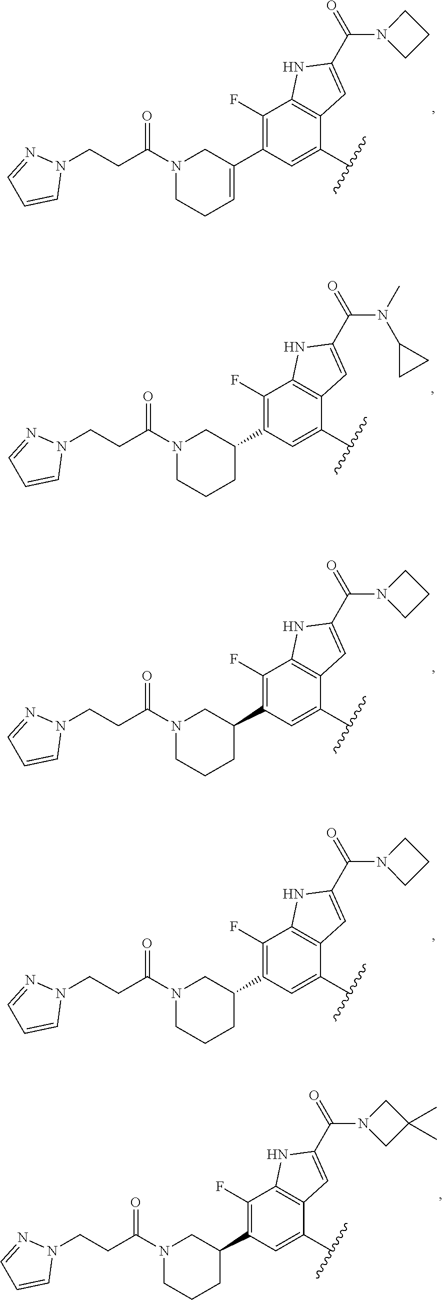 [SMILES: *c1cc(C2=CCCN(C(=O)CCn3cccn3)C2)c(F)c2[nH]c(C(=O)N3CCC3)cc12 ; *c1cc([C@@H]2CCCN(C(=O)CCn3cccn3)C2)c(F)c2[nH]c(C(=O)N(C)C3CC3)cc12 ; *c1cc([C@H]2CCCN(C(=O)CCn3cccn3)C2)c(F)c2[nH]c(C(=O)N3CCC3)cc12 ; *c1cc([C@@H]2CCCN(C(=O)CCn3cccn3)C2)c(F)c2[nH]c(C(=O)N3CCC3)cc12 ; *c1cc([C@H]2CCCN(C(=O)CCn3cccn3)C2)c(F)c2[nH]c(C(=O)N3CC(C)(C)C3)cc12]

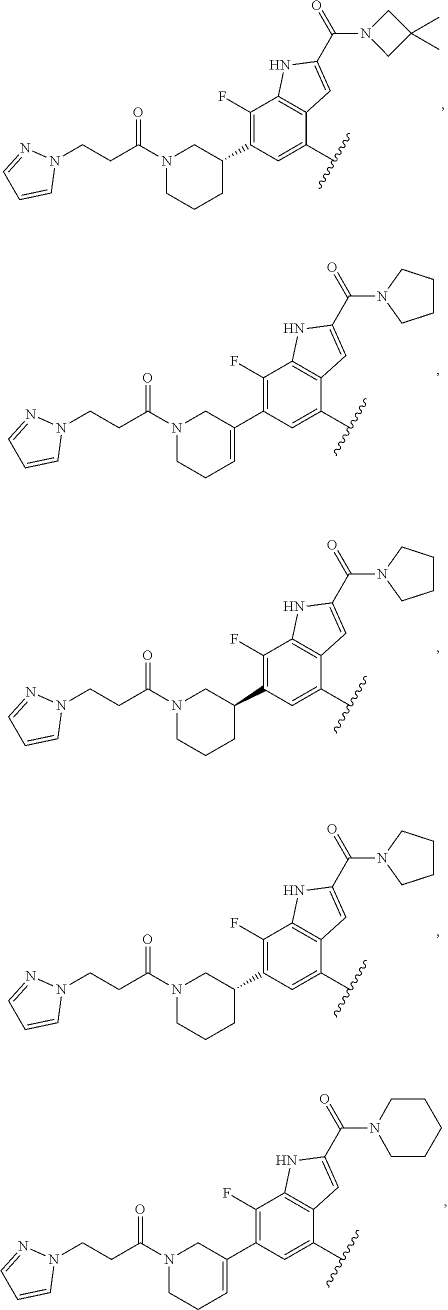 [SMILES: *c1cc([C@@H]2CCCN(C(=O)CCn3cccn3)C2)c(F)c2[nH]c(C(=O)N3CC(C)(C)C3)cc12 ; *c1cc(C2=CCCN(C(=O)CCn3cccn3)C2)c(F)c2[nH]c(C(=O)N3CCCC3)cc12 ; *c1cc([C@H]2CCCN(C(=O)CCn3cccn3)C2)c(F)c2[nH]c(C(=O)N3CCCC3)cc12 ; *c1cc([C@@H]2CCCN(C(=O)CCn3cccn3)C2)c(F)c2[nH]c(C(=O)N3CCCC3)cc12 ; *c1cc(C2=CCCN(C(=O)CCn3cccn3)C2)c(F)c2[nH]c(C(=O)N3CCCCC3)cc12]

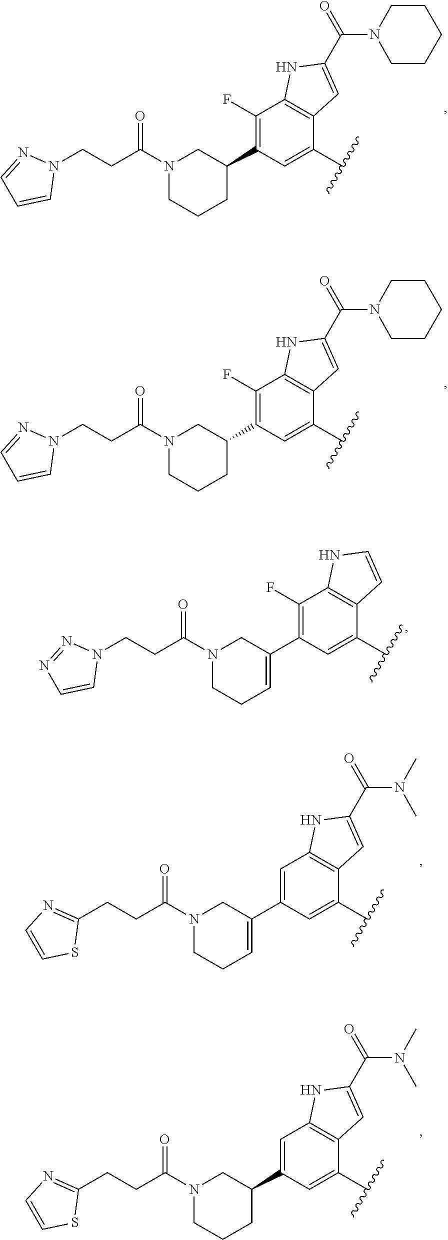 [SMILES: *c1cc([C@H]2CCCN(C(=O)CCn3cccn3)C2)c(F)c2[nH]c(C(=O)N3CCCCC3)cc12 ; *c1cc([C@@H]2CCCN(C(=O)CCn3cccn3)C2)c(F)c2[nH]c(C(=O)N3CCCCC3)cc12 ; *c1cc(C2=CCCN(C(=O)CCn3ccnn3)C2)c(F)c2[nH]ccc12 ; *c1cc(C2=CCCN(C(=O)CCc3nccs3)C2)cc2[nH]c(C(=O)N(C)C)cc12 ; *c1cc([C@H]2CCCN(C(=O)CCc3nccs3)C2)cc2[nH]c(C(=O)N(C)C)cc12]

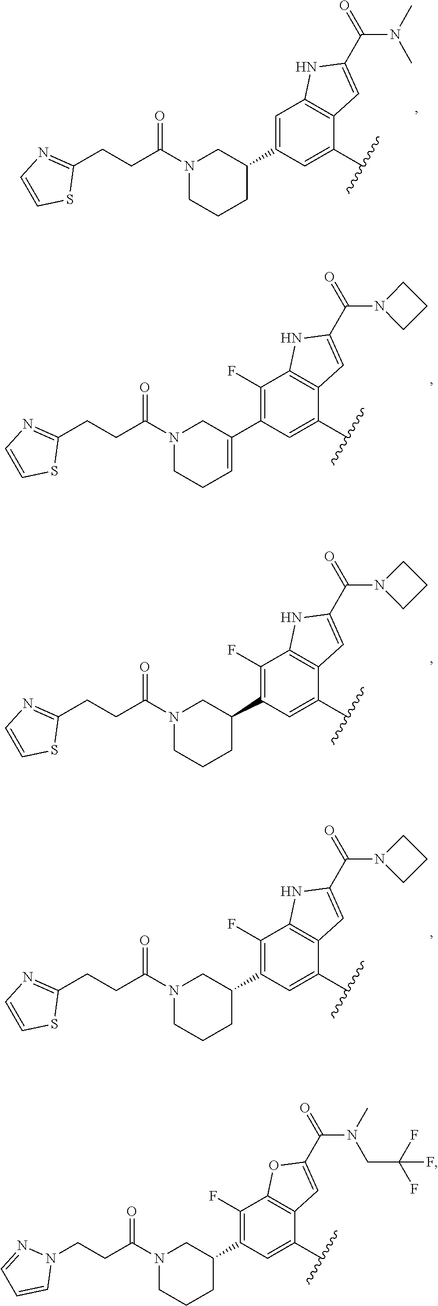 [SMILES: *c1cc([C@@H]2CCCN(C(=O)CCc3nccs3)C2)cc2[nH]c(C(=O)N(C)C)cc12 ; *c1cc(C2=CCCN(C(=O)CCc3nccs3)C2)c(F)c2[nH]c(C(=O)N3CCC3)cc12 ; *c1cc([C@H]2CCCN(C(=O)CCc3nccs3)C2)c(F)c2[nH]c(C(=O)N3CCC3)cc12 ; *c1cc([C@@H]2CCCN(C(=O)CCc3nccs3)C2)c(F)c2[nH]c(C(=O)N3CCC3)cc12 ; *c1cc([C@@H]2CCCN(C(=O)CCn3cccn3)C2)c(F)c2oc(C(=O)N(C)CC(F)(F)F)cc12]

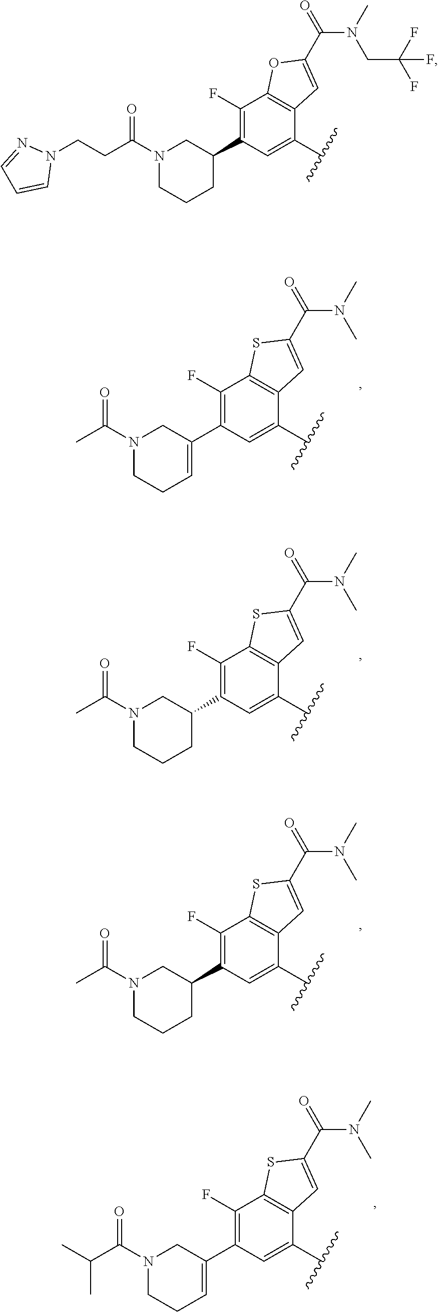

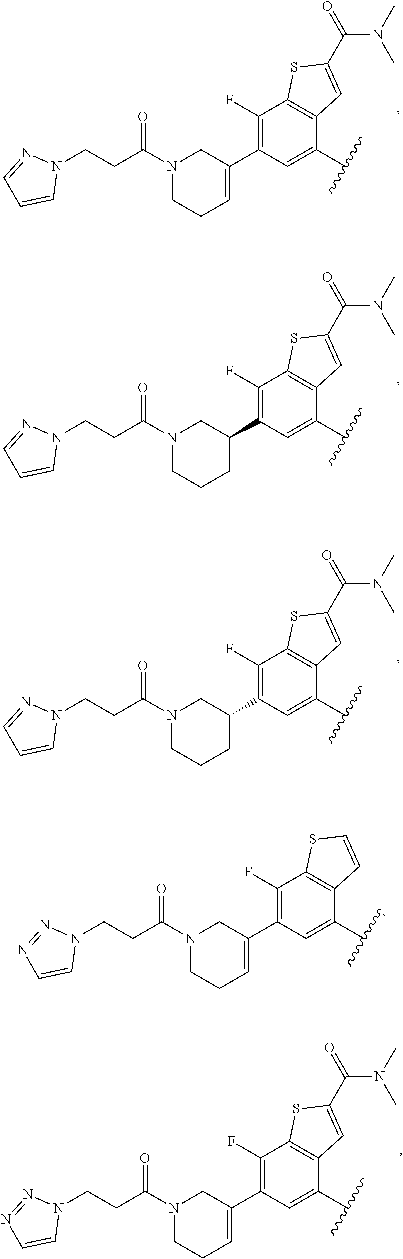 [SMILES: *c1cc(C2=CCCN(C(=O)CCn3cccn3)C2)c(F)c2sc(C(=O)N(C)C)cc12 ; *c1cc([C@H]2CCCN(C(=O)CCn3cccn3)C2)c(F)c2sc(C(=O)N(C)C)cc12 ; *c1cc([C@@H]2CCCN(C(=O)CCn3cccn3)C2)c(F)c2sc(C(=O)N(C)C)cc12 ; *c1cc(C2=CCCN(C(=O)CCn3ccnn3)C2)c(F)c2sccc12 ; *c1cc(C2=CCCN(C(=O)CCn3ccnn3)C2)c(F)c2sc(C(=O)N(C)C)cc12]

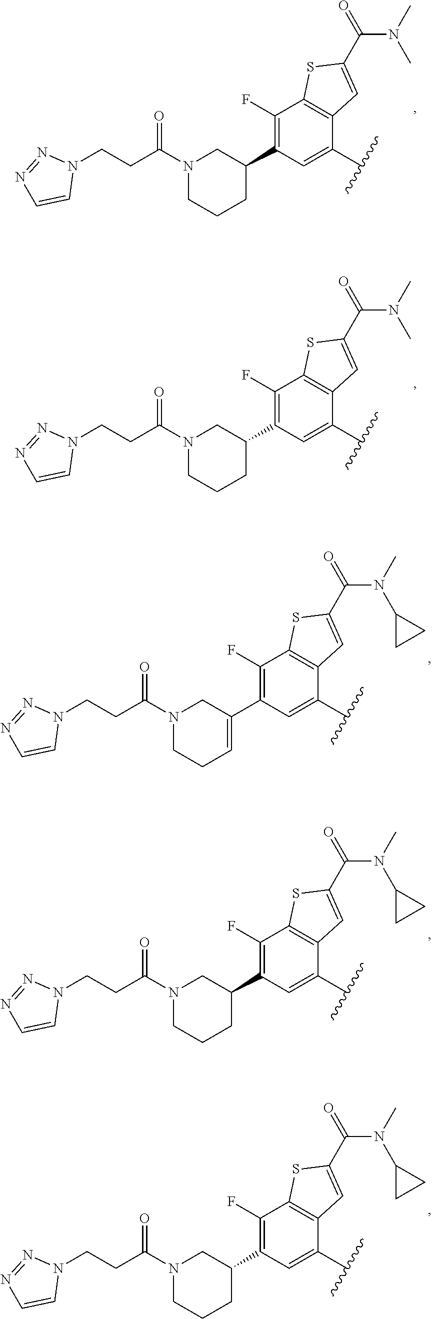 [SMILES: *c1cc([C@H]2CCCN(C(=O)CCn3ccnn3)C2)c(F)c2sc(C(=O)N(C)C)cc12 ; *c1cc([C@@H]2CCCN(C(=O)CCn3ccnn3)C2)c(F)c2sc(C(=O)N(C)C)cc12 ; *c1cc(C2=CCCN(C(=O)CCn3ccnn3)C2)c(F)c2sc(C(=O)N(C)C3CC3)cc12 ; *c1cc([C@H]2CCCN(C(=O)CCn3ccnn3)C2)c(F)c2sc(C(=O)N(C)C3CC3)cc12 ; *c1cc([C@@H]2CCCN(C(=O)CCn3ccnn3)C2)c(F)c2sc(C(=O)N(C)C3CC3)cc12]

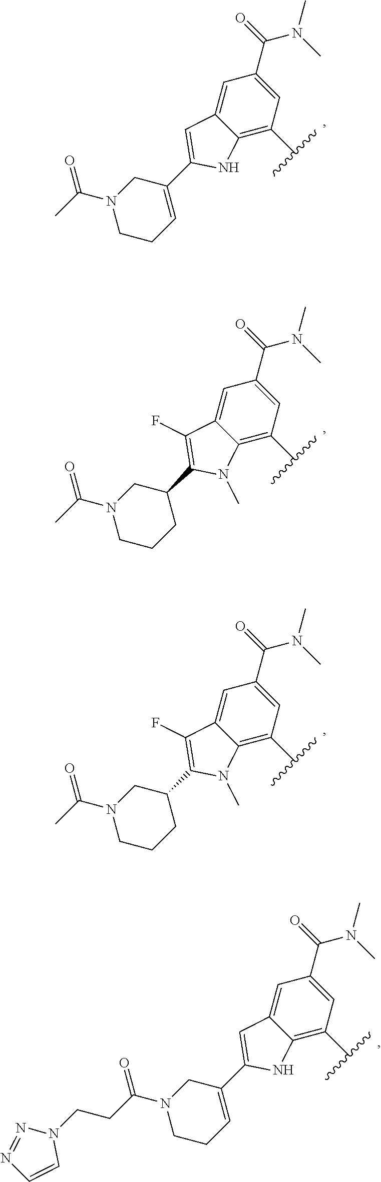

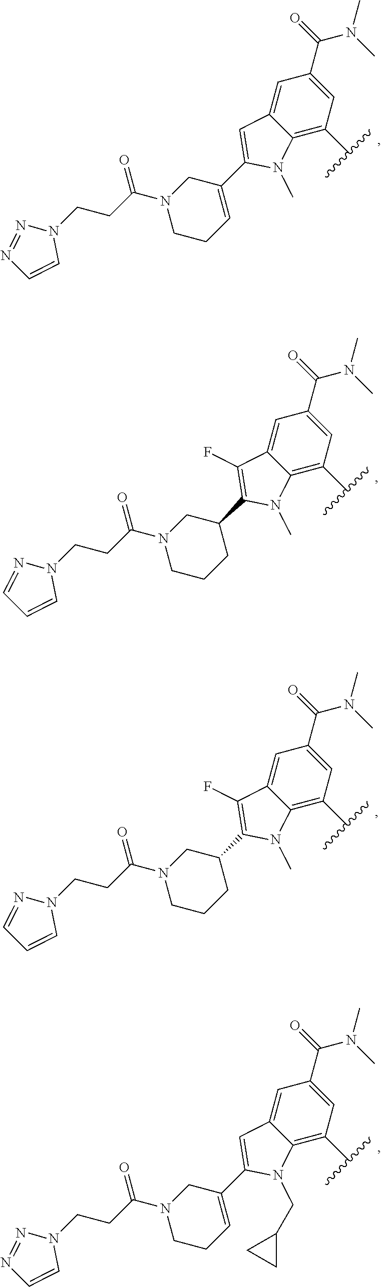 [SMILES: *c1cc(C(=O)N(C)C)cc2cc(C3=CCCN(C(=O)CCn4ccnn4)C3)n(C)c12 ; *c1cc(C(=O)N(C)C)cc2c(F)c([C@H]3CCCN(C(=O)CCn4cccn4)C3)n(C)c12 ; *c1cc(C(=O)N(C)C)cc2c(F)c([C@@H]3CCCN(C(=O)CCn4cccn4)C3)n(C)c12 ; *c1cc(C(=O)N(C)C)cc2cc(C3=CCCN(C(=O)CCn4ccnn4)C3)n(CC3CC3)c12]

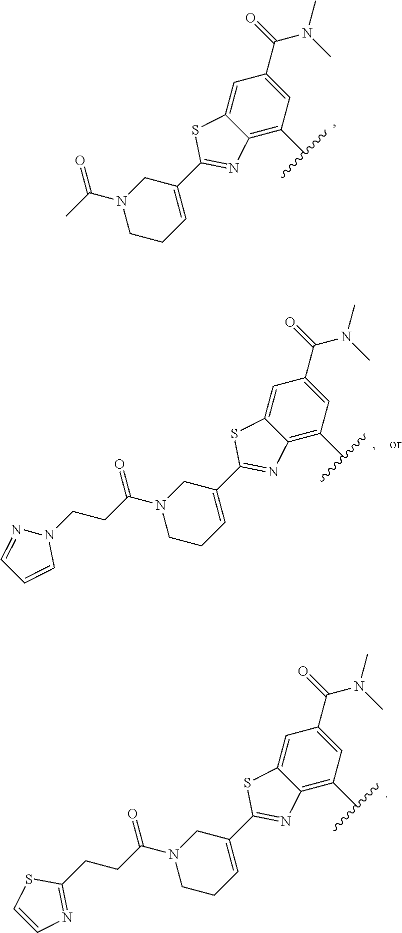 [SMILES: *c1cc(C(=O)N(C)C)cc2sc(C3=CCCN(C(C)=O)C3)nc12 ; *c1cc(C(=O)N(C)C)cc2sc(C3=CCCN(C(=O)CCn4cccn4)C3)nc12 ; *c1cc(C(=O)N(C)C)cc2sc(C3=CCCN(C(=O)CCc4nccs4)C3)nc12]

In some embodiments, SBM is

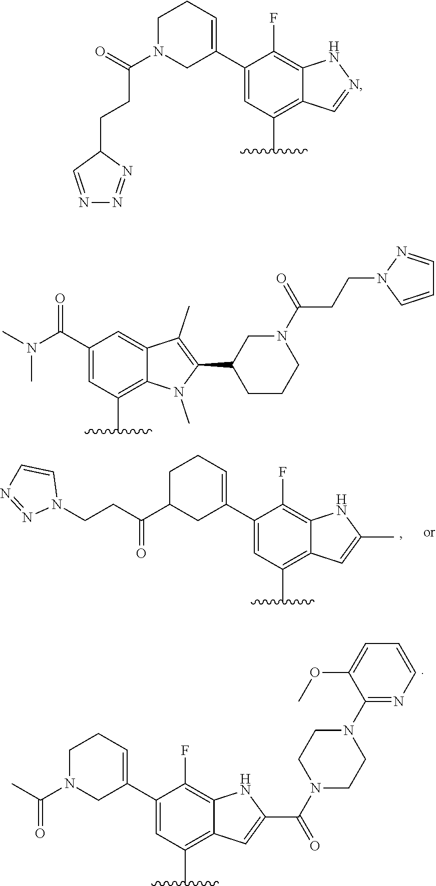

In some embodiments, SBM is as depicted in the compounds of Table 1, below.

In certain embodiments, the present invention provides a compound of formula I as a compound of any one of the following formulae:

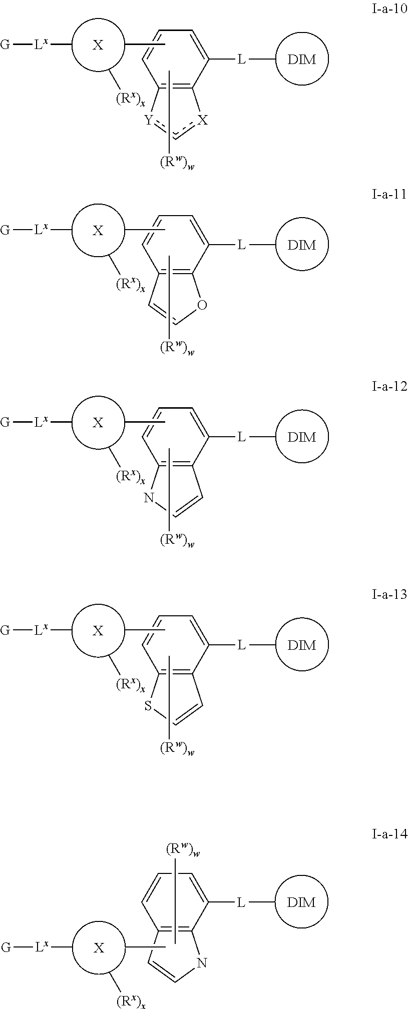

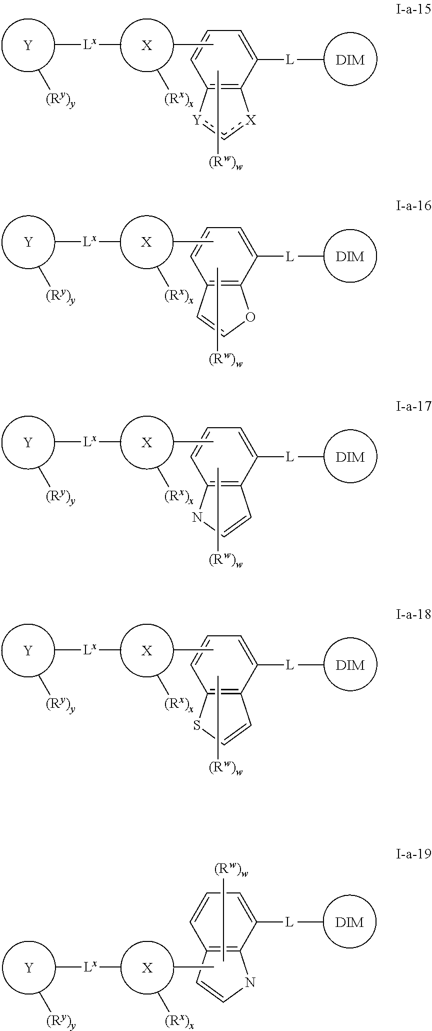

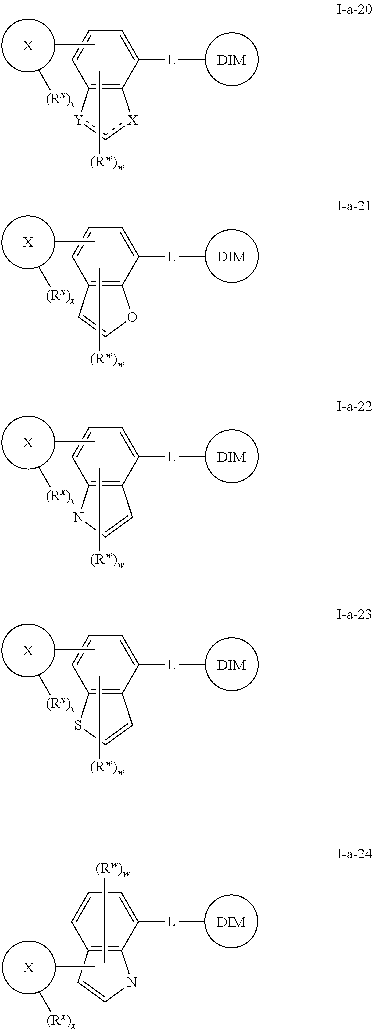

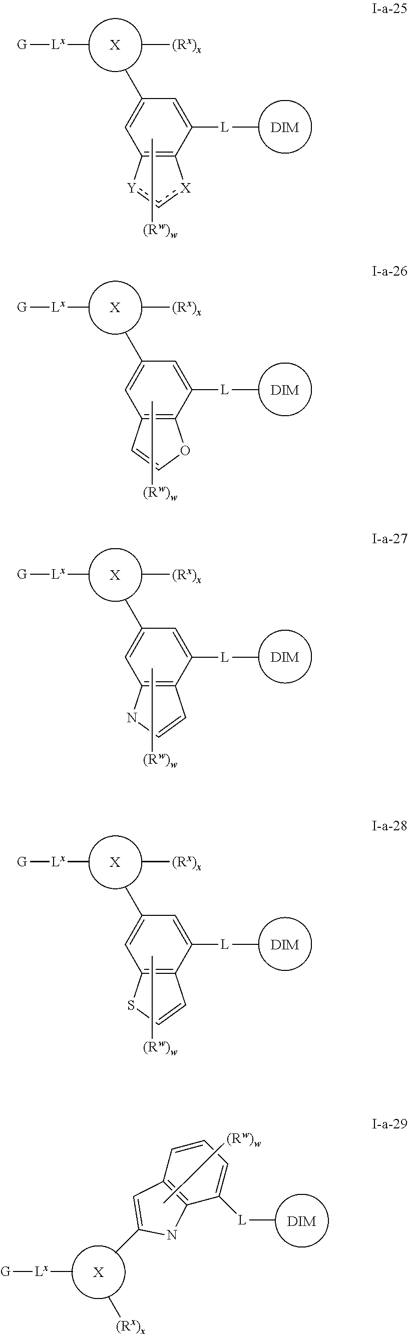

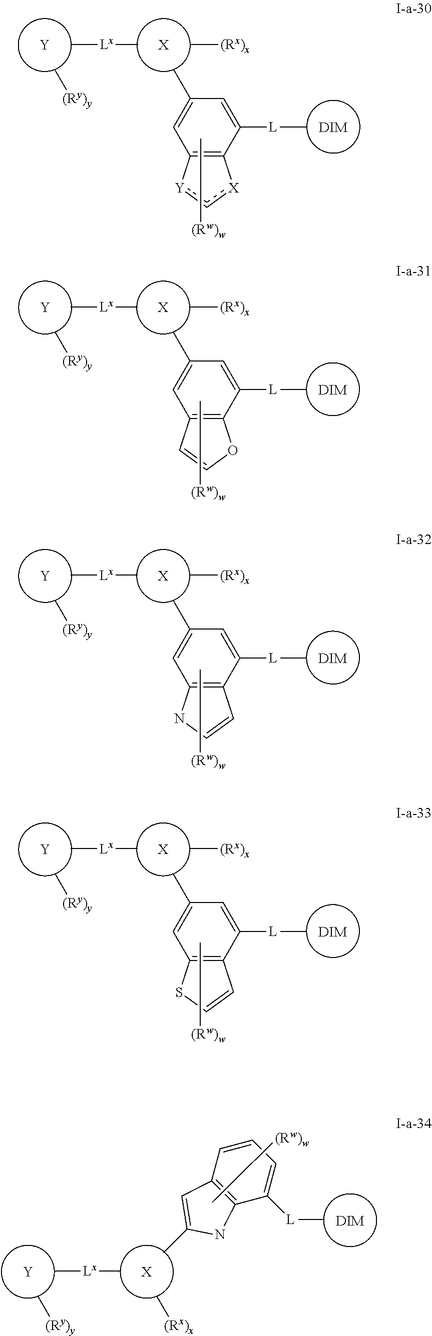

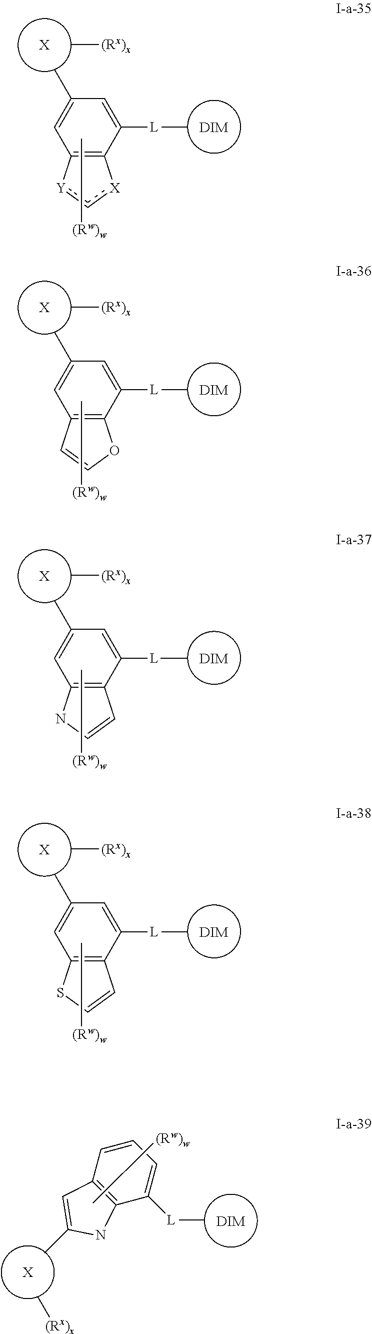

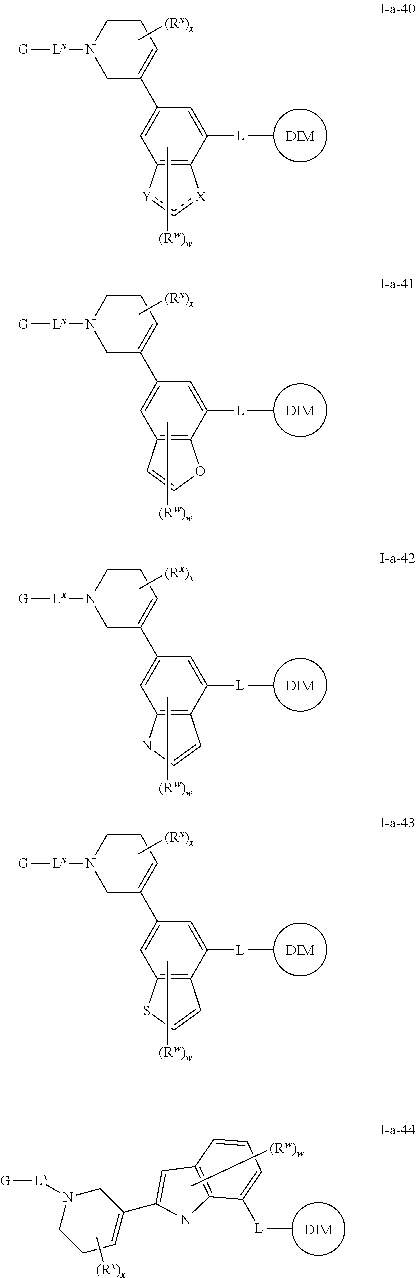

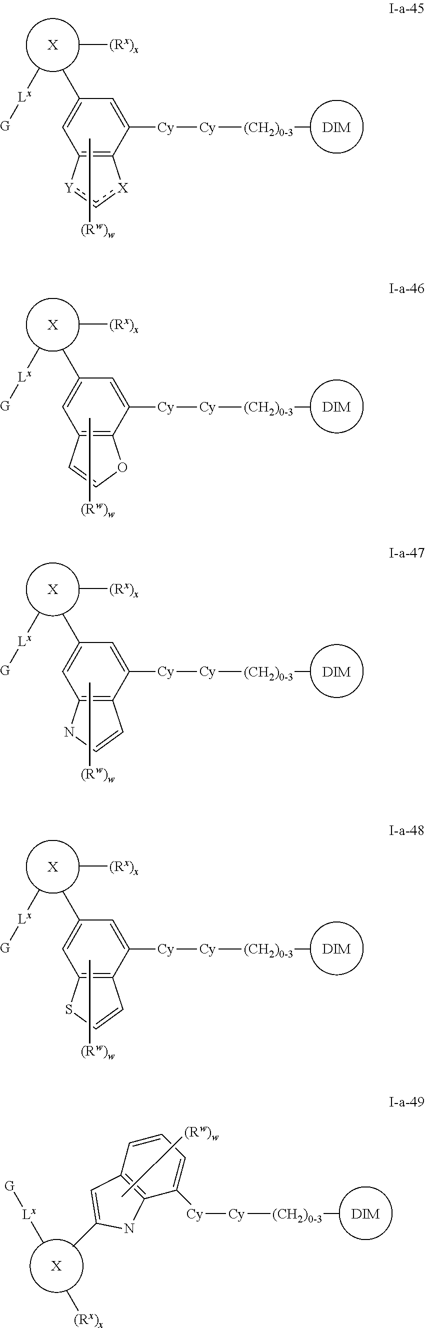

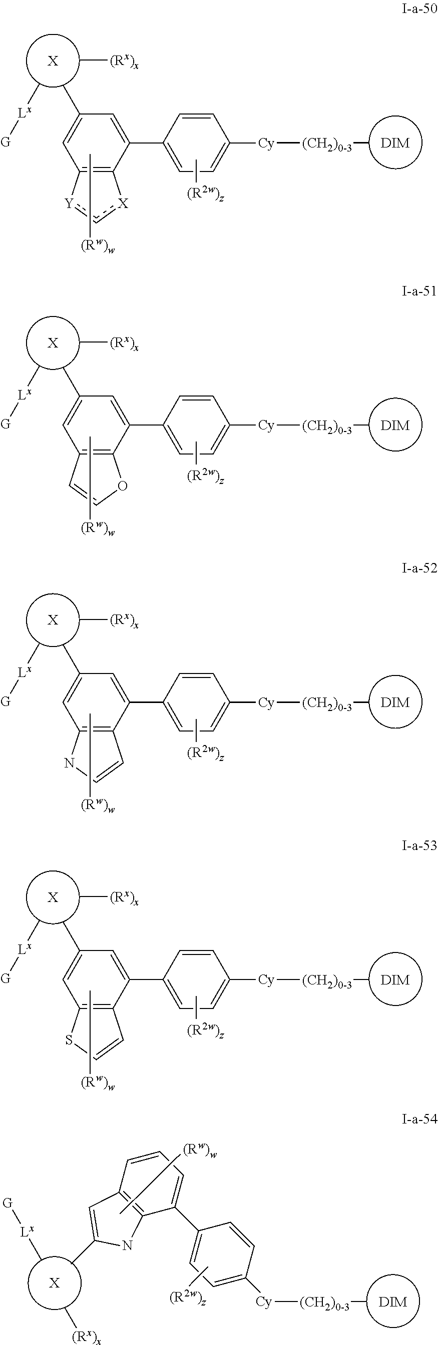

or a pharmaceutically acceptable salt thereof, wherein:
X is CH and Y is N—R^{w}, X is CH and Y is S, or X is O and Y is CH;
each R^{2w} is independently selected from hydrogen, R^{A}, halogen, —CN, —NO₂, —OR, —SR, —NR₂, —SiR₃, —S(O)R, —S(O)₂R, —S(O)(NR)R, —S(O)₂NR₂, —S(O)R, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R; and
z is 0, 1, 2, 3, or 4;
wherein DIM, L, -Cy-, Ring X, Ring Y, R, R^{w}, R^{x}, R^{y}, L^{x}, w, x, and y are as defined above and described herein both individually and in combination.

In some embodiments, X is CH and Y is N—R^{w}, X is CH and Y is S, or X is O and Y is CH.

In some embodiments, X is CH and Y is N—R^{w}. In some embodiments, X is CH and Y is NH. In some embodiments, X is CH and Y is S. In some embodiments, X is O and Y is CH.

In some embodiments, X and Y are as depicted in the compounds of Table 1, below.

As described above and defined herein, R^{2w} is selected from hydrogen, R^{A}, halogen, —CN, —NO₂, —OR, —SR, —NR₂, —SiR₃, —S(O)R, —S(O)₂R, —S(O)(NR)R, —S(O)₂NR₂, —S(O)R, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R.

In some embodiments, R^{2w} is hydrogen. In some embodiments, R^{2w} is R^{A}. In some embodiments, R^{2w} is halogen. In some embodiments, R^{2w} is —CN. In some embodiments, R^{2w} is —NO₂. In some embodiments, R^{2w} is —OR. In some embodiments, R^{2w} is —SR. In some embodiments, R^{2w} is —NR₂. In some embodiments, R^{2w} is —SiR₃. In some embodiments, R^{2w} is —S(O)₂R. In some embodiments. R^{2w} is —S(O)₂NR₂. In some embodiments, R^{2w} is —S(O)(NR)R. In some embodiments, R^{2w} is —S(O)R. In some embodiments, R^{2w} is —C(O)R. In some embodiments, R^{2w} is —C(O)OR. In some embodiments, R^{2w} is —C(O)NR₂. In some embodiments, R^{2w} is —C(O)NROR. In some embodiments, R^{2w} is —OC(O)R. In some embodiments, R^{2w} is —OC(O)NR₂. In some embodiments, R^{2w} is —P(O)R₂. In some embodiments, R^{2w} is —P(O)(OR)₂. In some embodiments, R^{2w} is —OP(O)R₂. In some embodiments, R^{2w} is —OP(O)(OR)₂. In some embodiments, R^{2w} is —OP(O)(OR)NR₂. In some embodiments, R^{2w} is —OP(O)(NR₂)₂. In some embodiments, R^{2w} is —NRC(O)OR. In some embodiments, R^{2w} is —NRC(O)R. In some embodiments, R^{2w} is —NRC(O)N(R)₂. In some embodiments, R^{2w} is —NP(O)R₂. In some embodiments, R^{2w} is —NRP(O)(OR)₂. In some embodiments, R^{2w} is —NRP(O)(OR)NR₂. In some embodiments, R^{2w} is —NRP(O)(NR₂)₂. In some embodiments, R^{2w} is —NRS(O)₂R.

In some embodiments, each R^{2w} is independently hydrogen, C₁₋₆ alkyl, C₁₋₆ haloalkyl, halogen, —OC₁₋₆ alkyl, or —OC₁₋₆ haloalkyl.

In some embodiments, each R^{2w} is independently fluoro, chloro, methyl, ethyl, —CHF₂, —CMeF₂, —CF₃, —OMe, —OEt, —OCHF₂, —OCMeF₂, or —OCF₃.

In some embodiments. R^{2w} is fluoro. In some embodiments, R^{2w} is chloro. In some embodiments, R^{2w} is fluoro and chloro. In some embodiments, R^{2w} is —OMe.

In some embodiments, R^{2w} is as depicted in the compounds of Table 1, below.

As described above and defined herein, z is 0, 1, 2, 3, or 4.

In some embodiments, z is 0. In some embodiments, z is 1. In some embodiments, z is 2. In some embodiments, z is 3. In some embodiments, z is 4. In some embodiments, z is 0 or 1. In some embodiments, z is 1 or 2.

In some embodiment, z is as depicted in the compounds of Table 1, below.

In certain embodiments, the present invention provides a compound of formula I-a as any one of the following formulae:

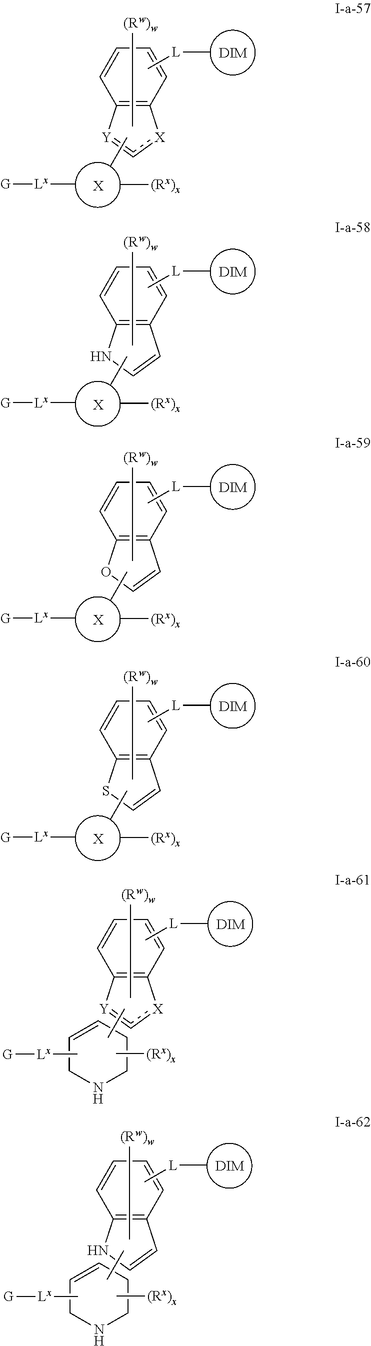

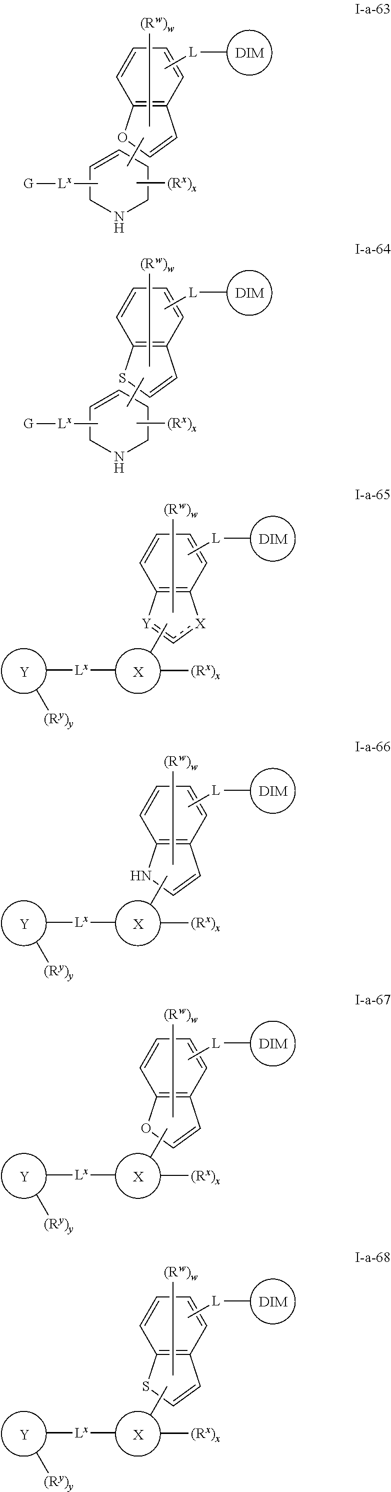

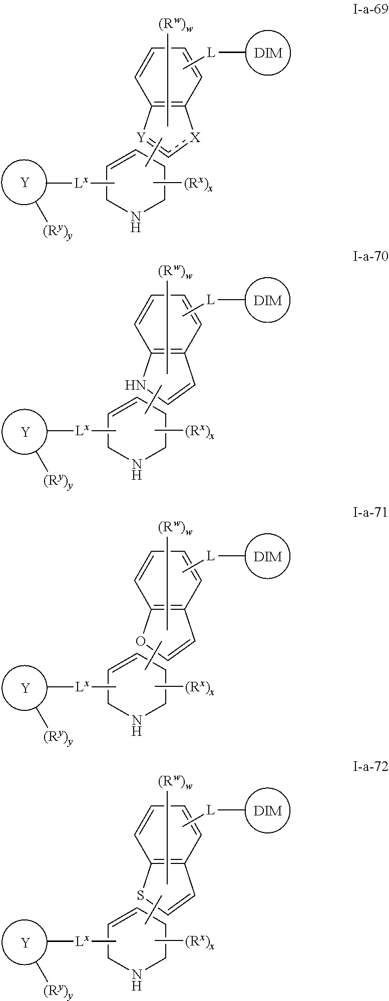

or a pharmaceutically acceptable salt thereof, wherein each of the variables are as defined above and described herein both individually and in combination, and wherein:
each of X and Y of Ring W is independently N, NH, N—R^{W}, —O—, —S—, C—H, C—R^{W}, C—H₂, CH(R^{W}), or C—(R^{W})₂.

### Ligase Binding Moiety (LBM)

In some embodiments, DIM is LBM. In some embodiments, LBM is an E3 ligase ligand. In some embodiments, LBM comprises means for binding an E3 ubiquitin ligase. In some embodiments, LBM comprises means for binding a cereblon E3 ubiquitin ligase. As defined herein and described below, wherein a formula is depicted using square brackets, e.g.,

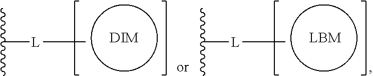

L is attached to a modifiable carbon, oxygen, or nitrogen atom within DIM or LBM including substitution or replacement of a defined group in DIM or LBM.

In certain embodiments, the present invention provides a compound of formula I, wherein LBM is a compound of formula I-aa:

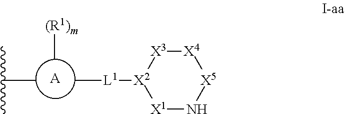

or a pharmaceutically acceptable salt thereof, wherein:
X¹ and X⁵ are independently a covalent bond, —CR₂—, —SO₂—, —S(O)—, —P(O)R—, —P(O)OR—, —P(O)N(R)₂—, —C(O)—, —C(S)—, or

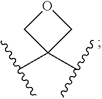 [SMILES: *C1(*)COC1]

X² is N, C—R^{B}, Si—R^{B}, or P═O;
X³ and X⁴ are independently a covalent bond, —CR₂—, —CF₂—, —O—, —S—, or X³—X⁴ is —CR═CR—;
each R¹ is independently R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)N(R)OR, —C(R)₂N(R)C(O)R, —C(R)₂N(R)C(O)NR₂, —OC(O)R, —OC(O)NR₂, —OP(O)R₂, —OP(O)(OR)₂, —OP(O)(OR)(NR₂), —OP(O)(NR₂)₂, —N(R)C(O)OR, —N(R)C(O)R, —N(R)C(O)NR₂, —N(R)S(O)₂R, —NP(O)R₂, —N(R)P(O)(OR)₂, —N(R)P(O)(OR)(NR₂), —N(R)P(O)(NR₂)₂, —N(R)S(O)₂R:
each R^{B} is independently, hydrogen, halogen, —CN, —OR, —SR, —S(O)R, —S(O)₂R, —NR₂, —P(O)(OR)₂, —P(O)(NR₂)OR, —P(O)(NR₂)₂, —Si(OH)₂R, —Si(OH)R₂, —SiR₃, or an optionally substituted C₁₋₄ aliphatic;
L¹ is a covalent bond or a C₁₋₃ bivalent hydrocarbon chain wherein 1-2 methylene units of the chain are independently and optionally replaced with —C(O)—, —C(S)—, —CR₂—, —CF₂—, —NR—, —O—, —S—, or —S(O)₂:
Ring A is phenylenyl, naphthalenyl, pyridinylenyl, a 4-7 membered saturated or partially unsaturated carbocyclylenyl, a 4-7 membered saturated or partially unsaturated heterocyclylenyl having 1-3 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, an 8-10 membered bicyclic heteroarylenyl having 1-5 heteroatoms independently selected from nitrogen, oxygen, and sulfur, an 8-15 membered saturated or partially unsaturated tricyclic heterocyclylenyl having 1-5 heteroatoms independently selected from nitrogen, oxygen, and sulfur, or an 8-15 membered tricyclic heteroarylenyl having 1-5 heteroatoms independently selected from nitrogen, oxygen, and sulfur;
each R^{A} is independently an optionally substituted group selected from C₁₋₆ aliphatic, phenyl, a 3-10 membered saturated or partially unsaturated carbocyclic ring, a 3-10 membered heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same or adjacent atoms or R^{B} and an R group are taken together with their intervening atoms to form an optionally substituted 3-11 membered saturated or partially unsaturated monocyclic, bicyclic, bridged bicyclic, or spirocyclic carbocyclic or heterocyclic ring having 0-3 heteroatoms independently selected from nitrogen, oxygen, and sulfur;
m is 0, 1, 2, 3, 4, or 5.

In certain embodiments, the present invention provides a compound of formula I, wherein LBM is of formula I-aa′:

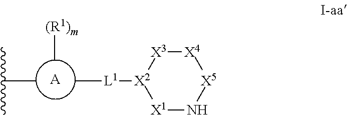

or a pharmaceutically acceptable salt thereof, wherein:
X¹ and X⁵ are independently a covalent bond, —CR₂—, —SO₂—, —S(O)—, —P(O)R—, —P(O)OR—, —P(O)N(R)₂—, —C(O)—, —C(S)—, or

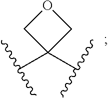 [SMILES: *C1(*)COC1]

X² is N, C—R^{B}, Si—R^{B}, or P═O;
X³ and X⁴ are independently a covalent bond, —CR₂—, —CF₂—, —O—, —S—, or X³—X⁴ is —CR═CR—;
each R¹ is independently —H, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)N(R)OR, —C(R)₂N(R)C(O)R, —C(R)₂N(R)C(O)NR₂, —OC(O)R, —OC(O)NR₂, —OP(O)R₂, —OP(O)(OR)₂, —OP(O)(OR)(NR₂), —OP(O)(NR₂)₂, —N(R)C(O)OR, —N(R)C(O)R, —N(R)C(O)NR₂, —N(R)S(O)₂R, —NP(O)R₂, —N(R)P(O)(OR)₂, —N(R)P(O)(OR)(NR₂), —N(R)P(O)(NR₂)₂, —N(R)S(O)₂R; or:
   two R¹ groups of Ring A are taken together with their intervening atoms to form an optionally substituted ring selected from a 3-10 membered saturated or partially unsaturated carbocyclyl or heterocyclyl having 1-4 heteroatoms independently selected from nitrogen, oxygen, or sulfur; benzo; or a 5-10 membered heteroaryl having 1-4 heteroatoms independently selected from nitrogen, oxygen, or sulfur;
each R^{B} is independently, hydrogen, halogen, —CN, —OR, —SR, —S(O)R, —S(O)₂R, —NR₂, —P(O)(OR)₂, —P(O)(NR₂)OR, —P(O)(NR₂)₂, —Si(OH)₂R, —Si(OH)R₂, —SiR₃, or an optionally substituted C₁₋₄ aliphatic;
L¹ is a covalent bond or a C₁₋₃ bivalent hydrocarbon chain wherein 1-2 methylene units of the chain are independently and optionally replaced with —C(O)—, —C(S)—, —CR₂—, —CF₂—, —NR—, —O—, —S—, or —S(O)₂;
Ring A is phenylenyl, naphthalenyl, pyridinylenyl, a 4-7 membered saturated or partially unsaturated carbocyclylenyl, a 4-7 membered saturated or partially unsaturated heterocyclylenyl having 1-3 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, an 8-10 membered bicyclic heteroarylenyl having 1-5 heteroatoms independently selected from nitrogen, oxygen, and sulfur, an 8-15 membered saturated or partially unsaturated tricyclic heterocyclylenyl having 1-5 heteroatoms independently selected from nitrogen, oxygen, and sulfur, or an 8-15 membered tricyclic heteroarylenyl having 1-5 heteroatoms independently selected from nitrogen, oxygen, and sulfur;
each R^{A} is independently an optionally substituted group selected from C₁₋₆ aliphatic, phenyl, a 3-10 membered saturated or partially unsaturated carbocyclic ring, a 3-10 membered heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same or adjacent atoms or R^{B} and an R group are taken together with their intervening atoms to form an optionally substituted 3-11 membered saturated or partially unsaturated monocyclic, bicyclic, bridged bicyclic, or spirocyclic carbocyclic or heterocyclic ring having 0-3 heteroatoms independently selected from nitrogen, oxygen, and sulfur.

In certain embodiments, the present invention provides a compound of formula I, wherein LBM is a compound of formula I-aa-1:

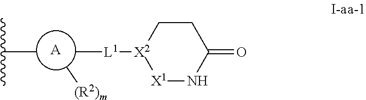

or a pharmaceutically acceptable salt thereof, wherein:
X¹ is a bivalent moiety selected from —CH₂— or —C(O)—;
X² is N or CH:
L¹ is a covalent bond or a C₁₋₃ bivalent hydrocarbon chain wherein 1-2 methylene units of the chain are independently and optionally replaced with —C(O)—, —C(S)—, —CR₂—, —CF₂—, —NR—, —O—, —S—, or —S(O)₂;
Ring A is a ring selected from phenylenyl, naphthalenyl, pyridinylenyl,

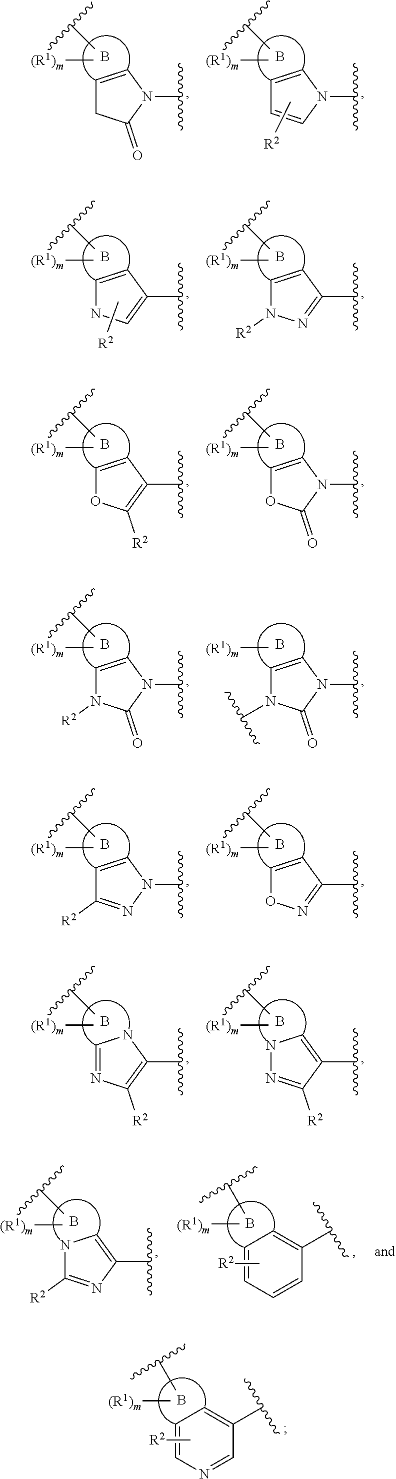

Ring B is a fused ring selected from benzo or a 5-6 membered heteroaryl containing 1-4 heteroatoms independently selected from nitrogen, oxygen, and sulfur;
each R¹ is independently R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)N(R)OR, —CR₂N(R)C(O)R, —CR₂N(R)C(O)NR₂, —CFR₂, —CF₂R, —CF₃, —CR₂(OR), —CR₂(NR₂), —OC(O)R, —OC(O)NR₂, —OP(O)R₂, —OP(O)(OR)₂, —OP(O)(OR)NR₂, —OP(O)(NR₂)₂, —N(R)C(O)OR, —N(R)C(O)R, —N(R)C(O)NR₂, —N(R)S(O)₂R, —N(R)P(O)R₂, —N(R)P(O)(OR)₂, —N(R)P(O)(OR)NR₂, —N(R)P(O)(NR₂)₂, or —N(R)S(O)₂R;
R² is hydrogen, halogen, C₁₋₆ alkyl, C₃₋₆cycloalkyl, C₁₋₆ haloalkyl, —OC₁₋₆ alkyl, —OC₃₋₆cycloalkyl, or —OC₁₋₆ haloalkyl;
each R^{A} is independently an optionally substituted group selected from C₁₋₆ aliphatic, phenyl, a 3-10 membered saturated or partially unsaturated carbocyclic ring, a 3-10 membered heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur:
each R is independently hydrogen, or an optionally substituted group selected from C₁₋₆ aliphatic, phenyl, a 3-7 membered saturated or partially unsaturated carbocyclic ring, a 3-7 membered heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same carbon or nitrogen are optionally taken together with their intervening atoms to form a 4-11 membered saturated or partially unsaturated monocyclic, bicyclic, bridged bicyclic, or spirocyclic carbocyclic or heterocyclic ring having 0-3 heteroatoms, in addition to the carbon or nitrogen from which the two R groups are attached, independently selected from nitrogen, oxygen, and sulfur; and
m is 0, 1, 2, 3 or 4.

In certain embodiments, the present invention provides a compound of formula I, wherein LBM is of formula I-aa-1′:

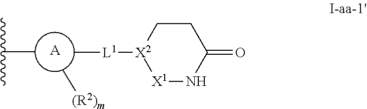

or a pharmaceutically acceptable salt thereof, wherein:
X¹ is a bivalent moiety selected from —CH₂— or —C(O)—;
X² is N or CH;
L¹ is a covalent bond or a C₁₋₃ bivalent hydrocarbon chain wherein 1-2 methylene units of the chain are independently and optionally replaced with —C(O)—, —C(S)—, —CR₂—, —CF₂—, —NR—, —O—, —S—, or —S(O)₂;
Ring A is a ring selected from phenylenyl, naphthalenyl, pyridinylenyl,

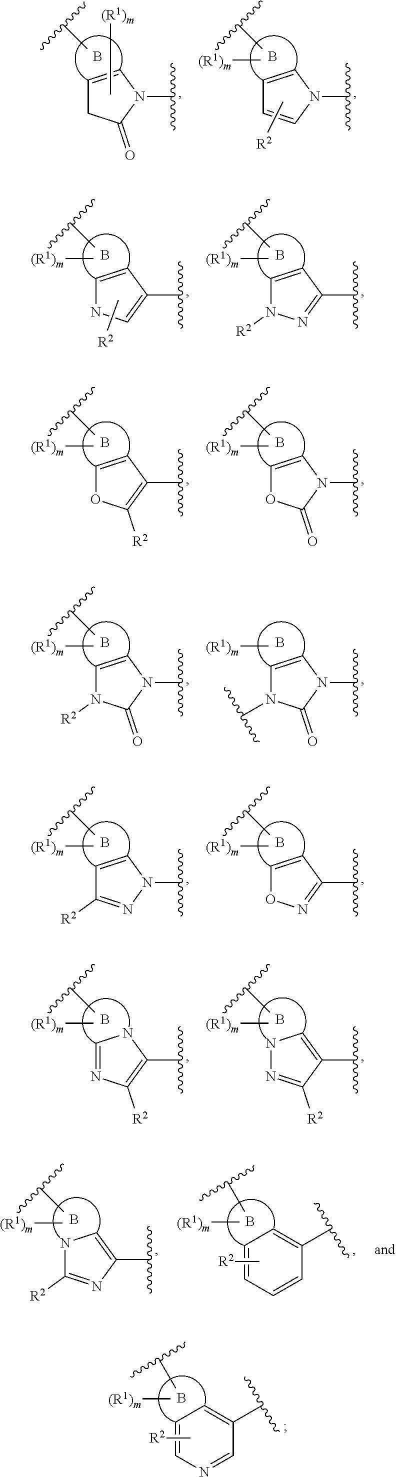

Ring B is a fused ring selected from benzo, a saturated or partially unsaturated 4-7 membered carbocyclyl, a saturated or partially unsaturated 4-7 membered heterocyclyl having 1-2 heteroatoms independently selected from nitrogen, oxygen, and sulfur, and a 5-6 membered heteroaryl containing 1-4 heteroatoms independently selected from nitrogen, oxygen, and sulfur;
each R¹ is independently R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)N(R)OR, —CR₂N(R)C(O)R, —CR₂N(R)C(O)NR₂, —CFR₂, —CF₂R, —CF₃, —CR₂(OR), —CR₂(NR₂), —OC(O)R, —OC(O)NR₂, —OP(O)R₂, —OP(O)(OR)₂, —OP(O)(OR)NR₂, —OP(O)(NR₂)₂, —N(R)C(O)OR, —N(R)C(O)R, —N(R)C(O)NR₂, —N(R)S(O)₂R, —N(R)P(O)R₂, —N(R)P(O)(OR)₂, —N(R)P(O)(OR)NR₂, —N(R)P(O)(NR₂)₂, or —N(R)S(O)₂R; or:
   two R¹ groups of Ring A are taken together with their intervening atoms to form an optionally substituted ring selected from a 3-10 membered saturated or partially unsaturated carbocyclyl or heterocyclyl having 1-4 heteroatoms independently selected from nitrogen, oxygen, or sulfur; benzo; or a 3-10 membered heteroaryl having 1-4 heteroatoms independently selected from nitrogen, oxygen, or sulfur:
R² is hydrogen, halogen, C₁₋₆ alkyl, C₃₋₆cycloalkyl, C₁₋₆ haloalkyl, —OC₁₋₆ alkyl, —OC₃₋₆cycloalkyl, or —OC₁₋₆ haloalkyl; or:
   an R² and an R¹ are taken together with their intervening atoms to form an optionally substituted ring selected from a 3-10 membered saturated or partially unsaturated carbocyclyl or heterocyclyl having 1-4 heteroatoms independently selected from nitrogen, oxygen, or sulfur; benzo; or a 5-10 membered heteroaryl having 1-4 heteroatoms independently selected from nitrogen, oxygen, or sulfur:
each R^{A} is independently an optionally substituted group selected from C₁₋₆ aliphatic, phenyl, a 3-10 membered saturated or partially unsaturated carbocyclic ring, a 3-10 membered heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 3-7 membered saturated or partially unsaturated carbocyclic ring, a 3-7 membered heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same carbon or nitrogen are optionally taken together with their intervening atoms to form a 4-11 membered saturated or partially unsaturated monocyclic, bicyclic, bridged bicyclic, or spirocyclic carbocyclic or heterocyclic ring having 0-3 heteroatoms, in addition to the carbon or nitrogen from which the two R groups are attached, independently selected from nitrogen, oxygen, and sulfur; and
m is 0, 1, 2, 3 or 4.

For formula I-aa-1 and I-aa-1′, it will be appreciated that an occurrence of R² reduces the available occurrences of m by 1.

In some embodiments, the present invention provides a compound of formula I-aa-1 as a compound of any one of the following formulae:

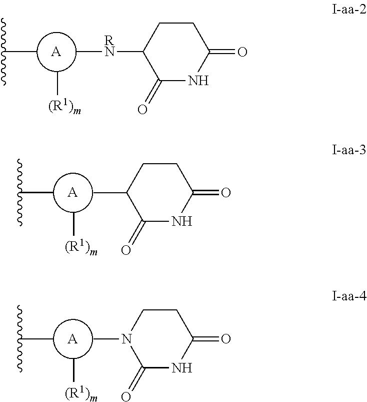

or a pharmaceutically acceptable salt thereof.

In some embodiments, the present invention provides a compound of formula I-aa′ or I-aa-1′ as a compound of any one of the following formulae:

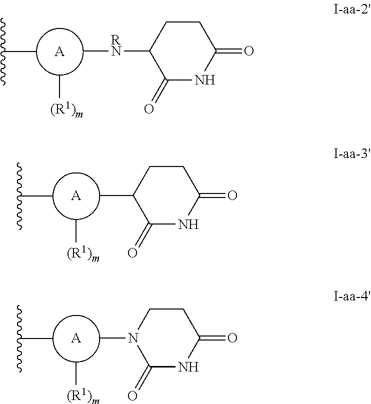

or a pharmaceutically acceptable salt thereof, wherein each R¹, m, Ring A, and R is as defined above in I-aa′ or I-aa-1′ and described herein both individually and in combination.

In some embodiments, the present invention provides a compound of formula I-aa′ or I-aa-1′ as a compound of any one of the following formulae:

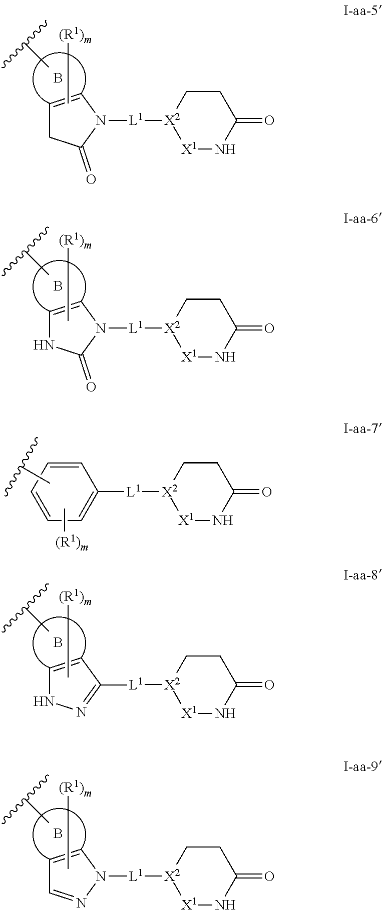

or a pharmaceutically acceptable salt thereof, wherein each L¹, R¹, m, X¹, X², and Ring B is as defined above in I-aa′ or I-aa-1′ and described herein both individually and in combination.

In some embodiments, the present invention provides a compound of formula I-aa′ or I-aa-1′ as a compound of any one of the following formulae:

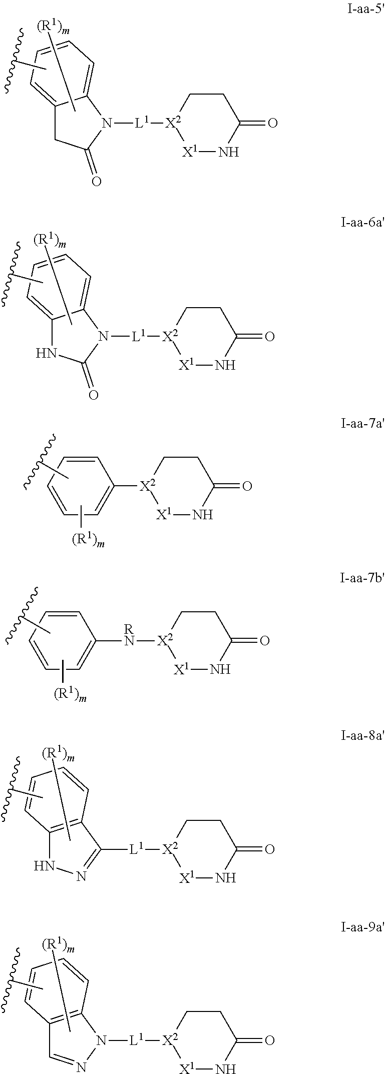

or a pharmaceutically acceptable salt thereof, wherein each L¹, R¹, m, X¹, and X² is as defined above in I-aa′ or I-aa-1′ and described herein both individually and in combination.

In some embodiments, the present invention provides a compound of formula I-aa′ or I-aa-1′ as a compound of any one of the following formulae:

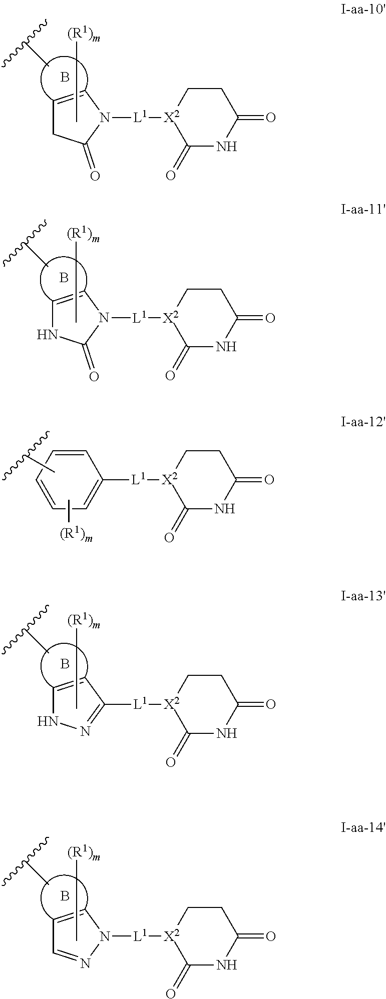

or a pharmaceutically acceptable salt thereof, wherein each variable is as defined above and described herein both individually and in combination.

In some embodiments, the present invention provides a compound of formula I-aa′ or I-aa-1′ as a compound of any one of the following formulae:

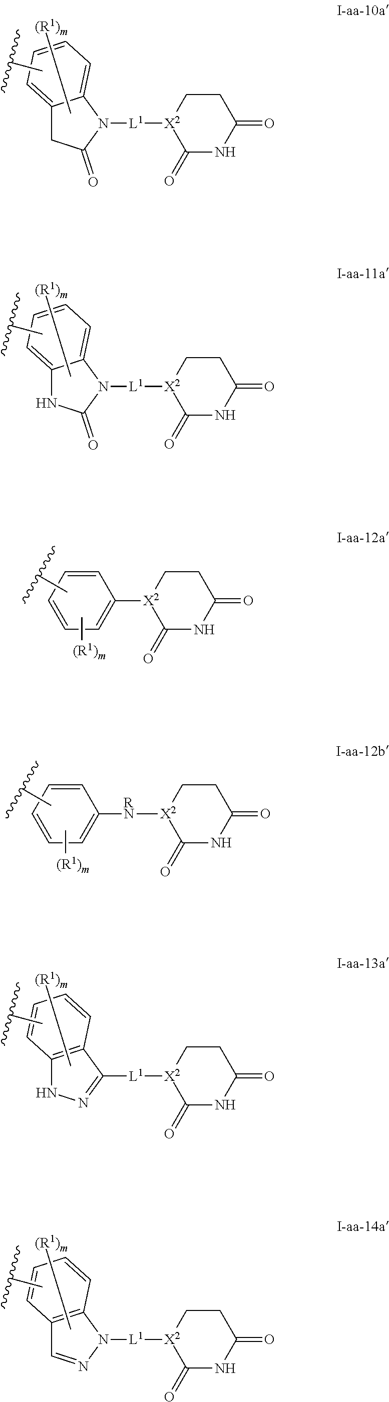

or a pharmaceutically acceptable salt thereof, wherein each variable is as defined above and described herein both individually and in combination.

As defined above and described herein, X¹ and X⁵ are independently a covalent bond, —CR₂—, —SO₂—, —S(O)—, —P(O)R—, —P(O)OR—, —P(O)N(R)₂—, —C(O)—, —C(S)—, or

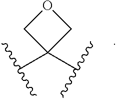 [SMILES: *C1(*)COC1]

In some embodiments, X¹ is a covalent bond. In some embodiments, X¹ is —CR₂—. In some embodiments, X¹ is —SO₂—. In some embodiments, X¹ is —S(O)—. In some embodiments, X¹ is —P(O)R—. In some embodiments, X¹ is —P(O)OR—. In some embodiments, X¹ is —P(O)N(R)₂—. In some embodiments, X is —C(O)—. In some embodiments, X¹ is —C(S)—, or

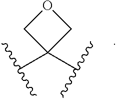 [SMILES: *C1(*)COC1]

In some embodiments, X¹ is —CH₂—. In some embodiments, X¹ is —C(O)—.

In some embodiments, X¹ is selected from those depicted in the compounds of Table 1 below.

In some embodiments, X⁵ is a covalent bond. In some embodiments, X⁵ is —CR₂—. In some embodiments, X⁵ is —SO₂—. In some embodiments, X⁵ is —S(O)—. In some embodiments, X⁵ is —P(O)R—. In some embodiments, X⁵ is —P(O)OR—. In some embodiments, X⁵ is —P(O)N(R)₂—. In some embodiments, X⁵ is —C(O)—. In some embodiments, X⁵ is —C(S)—, or

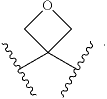 [SMILES: *C1(*)COC1]

In some embodiments, X⁵ is —CH₂—. In some embodiments, X⁵ is —C(O)—.

In some embodiments, X is selected from those depicted in the compounds of Table 1 below.

As defined above and described herein, X is N. C—R^{B}, Si—R^{B}, or P═O. In some embodiments, X² is N. In some embodiments, X² is C—R^{B}. In some embodiments, X² is Si—R^{B}. In some embodiments, X² is P═O. In some embodiments, X² is CH.

In some embodiments, X² is selected from those depicted in the compounds of Table 1 below.

As defined above and described herein, X³ and X⁴ are independently a covalent bond, —CR₂—. —CF₂—, —O—, —S—, or X³—X⁴ is —CR═CR—. In some embodiments, X³ is —CR₂—. In some embodiments, X³ is —CF₂—. In some embodiments, X³ is

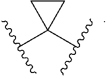 [SMILES: *C1(*)CC1]

In some embodiments, X³ is —O—. In some embodiments, X³ is —S—.

In some embodiments, X³ is selected from those depicted in the compounds of Table 1 below.

In some embodiments, X⁴ is —CR₂—. In some embodiments, X⁴ is —CF₂—. In some embodiments, X⁴ is

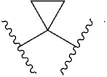 [SMILES: *C1(*)CC1]

In some embodiments, X⁴ is —O—. In some embodiments, X⁴ is —S—.

In some embodiments, X³—X⁴ is —CR═CR—.

In some embodiments, X⁴ is selected from those depicted in the compounds of Table 1 below.

As defined above and described herein, each R¹ is independently R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)N(R)OR, —C(R)₂N(R)C(O)R, —C(R)₂N(R)C(O)NR₂, —OC(O)R, —OC(O)NR₂, —OP(O)R₂, —OP(O)(OR)₂, —OP(O)(OR)(NR₂), —OP(O)(NR₂)₂, —N(R)C(O)OR, —N(R)C(O)R, —N(R)C(O)NR₂, —N(R)S(O)₂R, —NP(O)R₂, —N(R)P(O)(OR)₂, —N(R)P(O)(OR)(NR₂), —N(R)P(O)(NR₂)₂, —N(R)S(O)₂R; or: two R¹ groups on the same or adjacent atoms are taken together with their intervening atoms to form an optionally substituted ring selected from a 3-10 membered saturated or partially unsaturated carbocyclyl or heterocyclyl having 1-4 heteroatoms independently selected from nitrogen, oxygen, or sulfur; benzo; or a 5-10 membered heteroaryl having 1-4 heteroatoms independently selected from nitrogen, oxygen, or sulfur. In some embodiments, each R¹ is independently R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)N(R)OR, —C(R)₂N(R)C(O)R, —C(R)₂N(R)C(O)NR₂, —OC(O)R, —OC(O)NR₂, —OP(O)R₂, —OP(O)(OR)₂, —OP(O)(OR)(NR₂), —OP(O)(NR₂)₂, —N(R)C(O)OR, —N(R)C(O)R, —N(R)C(O)NR₂, —N(R)S(O)₂R, —NP(O)R₂, —N(R)P(O)(OR)₂, —N(R)P(O)(OR)(NR₂), —N(R)P(O)(NR₂)₂, —N(R)S(O)₂R.

In some embodiments, each R¹ is independently R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)N(R)OR, —C(R)₂N(R)C(O)R, —C(R)₂N(R)C(O)NR₂, —OC(O)R, —OC(O)NR₂, —OP(O)R₂, —OP(O)(OR)₂, —OP(O)(OR)(NR₂), —OP(O)(NR₂)₂, —N(R)C(O)OR, —N(R)C(O)NR₂, —N(R)S(O)₂R, —NP(O)R₂, —N(R)P(O)(OR)₂, —N(R)P(O)(OR)(NR₂), —N(R)P(O)(NR₂)₂, —N(R)S(O)₂R; or: two R¹ groups on the same or adjacent atoms are taken together with their intervening atoms to form an optionally substituted ring selected from a 3-10 membered saturated or partially unsaturated carbocyclyl or heterocyclyl having 1-4 heteroatoms independently selected from nitrogen, oxygen, or sulfur; benzo; or a 5-10 membered heteroaryl having 1-4 heteroatoms independently selected from nitrogen, oxygen, or sulfur.

In some embodiments, R¹ is R^{A}. In some embodiments, R¹ is —CN. In some embodiments, R¹ is —NO₂. In some embodiments, R¹ is —OR. In some embodiments, one or more of R¹ is —Si(OH)₂R. In some embodiments, R¹ is —Si(OH)R₂. In some embodiments, R¹ is —SR. In some embodiments, R¹ is —NR₂. In some embodiments. R¹ is —SiR₃. In some embodiments. R¹ is —S(O)₂R. In some embodiments, R¹ is —S(O)₂NR₂. In some embodiments, R¹ is —S(O)R. In some embodiments, R¹ is —C(O)R. In some embodiments, R¹ is —C(O)OR. In some embodiments, R¹ is —C(O)NR₂. In some embodiments, R¹ is —C(O)N(R)OR. In some embodiments, R¹ is —CR₂N(R)C(O)R. In some embodiments, R¹ is —CR₂N(R)C(O)NR₂. In some embodiments, R¹ is —CFR₂. In some embodiments, R¹ is —CF₂R. In some embodiments, R¹ is —CF₃. In some embodiments, R¹ is —CR₂(OR). In some embodiments, R¹ is —CR₂(NR₂). In some embodiments, R¹ is —OC(O)R. In some embodiments, R¹ is —OC(O)NR₂. In some embodiments, R¹ is —OP(O)R². In some embodiments, R¹ is —OP(O)(OR)₂. In some embodiments, R¹ is —OP(O)(OR)NR₂. In some embodiments, R¹ is independently —OP(O)(NR₂)₂—. In some embodiments, R¹ is —N(R)C(O)OR. In some embodiments, R¹ is —N(R)C(O)R. In some embodiments, R¹ is —N(R)C(O)NR₂. In some embodiments, R¹ is —N(R)P(O)R₂. In some embodiments, R¹ is —N(R)P(O)(OR)₂. In some embodiments, R¹ is —N(R)P(O)(OR)NR₂. In some embodiments, R¹ is —N(R)P(O)(NR₂)₂. In some embodiments, R¹ is —N(R)S(O)₂R.

In some embodiments, R¹ is halogen, C₁₋₆alkyl, —OC₁₋₆alkyl, C₁₋₆ haloalkyl, —OC₁₋₆ alkyl, or —OC₁₋₆ haloalkyl.

In some embodiments, R¹ is hydrogen. In some embodiments, R¹ is fluoro. In some embodiments, R¹ is chloro. In some embodiments, R¹ is methyl. In some embodiments. R¹ is —C(OH)Me₂. In some embodiments. R¹ is —CHF₂. In some embodiments, R¹ is —CF₃. In some embodiments. R¹ is —OMe.

In some embodiments, two R¹ groups of Ring A are taken together with their intervening atoms to form an optionally substituted ring selected from a 3-10 membered saturated or partially unsaturated carbocyclyl or heterocyclyl having 1-4 heteroatoms independently selected from nitrogen, oxygen, or sulfur; benzo; or a 5-10 membered heteroaryl having 1-4 heteroatoms independently selected from nitrogen, oxygen, or sulfur.

In some embodiments, two R¹ groups of Ring A are taken together with their intervening atoms to form an optionally substituted ring selected from a 3-10 membered saturated or partially unsaturated carbocyclyl. In some embodiments, two R¹ groups on the same or adjacent atoms are taken together with their intervening atoms to form an optionally substituted ring selected from a 5-6 membered saturated or partially unsaturated carbocyclyl.

In some embodiments, two R¹ groups of Ring A are taken together with their intervening atoms to form an optionally substituted ring selected from a 3-10 membered saturated or partially unsaturated heterocyclyl having 1-4 heteroatoms independently selected from nitrogen, oxygen, or sulfur. In some embodiments, two R¹ groups of Ring A are taken together with their intervening atoms to form an optionally substituted ring selected from a 4-7 membered saturated or partially unsaturated heterocyclyl having 1-4 heteroatoms independently selected from nitrogen, oxygen, or sulfur. In some embodiments, two R¹ groups of Ring A are taken together with their intervening atoms to form an optionally substituted ring selected from a 5-6 membered saturated or partially unsaturated heterocyclyl having 1-4 heteroatoms independently selected from nitrogen, oxygen, or sulfur.

In some embodiments, two R¹ groups of Ring A are taken together with their intervening atoms to form an optionally substituted benzo.

In some embodiments, two R¹ groups of Ring A are taken together with their intervening atoms to form an optionally substituted ring selected from a 5-10 membered heteroaryl having 1-4 heteroatoms independently selected from nitrogen, oxygen, or sulfur. In some embodiments, two R¹ groups of Ring A are taken together with their intervening atoms to form an optionally substituted ring selected from a 5-6 membered heteroaryl having 1-4 heteroatoms independently selected from nitrogen, oxygen, or sulfur.

In some embodiments, two R¹ groups on the same atom of Ring A are taken together to form an optionally substituted ring selected from a 3-10 membered saturated or partially unsaturated carbocyclyl or heterocyclyl having 1-4 heteroatoms independently selected from nitrogen, oxygen, or sulfur; benzo. In some embodiments, two R¹ groups on the same atom of Ring A are taken together to form an optionally substituted ring selected from a 3-10 membered saturated or partially unsaturated carbocyclyl. In some embodiments, two R¹ groups on the same atom of Ring A are taken together to form an optionally substituted ring selected from a 3-6 membered saturated or partially unsaturated carbocyclyl. In some embodiments, two R¹ groups on the same atom of Ring A are taken together to form an optionally substituted ring selected from a 3 membered saturated or partially unsaturated carbocyclyl. In some embodiments, two R¹ groups on the same atom of Ring A are taken together to form an optionally substituted ring selected from a 3-10 membered saturated or partially unsaturated heterocyclyl having 1-4 heteroatoms independently selected from nitrogen, oxygen, or sulfur. In some embodiments, two R¹ groups on the same atom of Ring A are taken together to form an optionally substituted ring selected from a 3-6 membered saturated or partially unsaturated heterocyclyl having 1-4 heteroatoms independently selected from nitrogen, oxygen, or sulfur. In some embodiments, two R¹ groups on the same atom of Ring A are taken together to form an optionally substituted ring selected from a 3 membered saturated or partially unsaturated heterocyclyl having 1-4 heteroatoms independently selected from nitrogen, oxygen, or sulfur. In some embodiments, two R¹ groups on the same atom of Ring A are taken together to form an optionally substituted cyclopropyl ring.

In some embodiments, R¹ is selected from those depicted in the compounds of Table 1 below.

As defined above and described herein, R² is hydrogen, halogen, C₁₋₆ alkyl, C₃₋₆cycloalkyl, C₁₋₆ haloalkyl, —OC₁₋₆ alkyl, —OC₃₋₆cycloalkyl, or —OC₁₋₆ haloalkyl; or: two R² groups are taken together with their intervening atoms to form an optionally substituted ring selected from a 3-10 membered saturated or partially unsaturated carbocyclyl or heterocyclyl having 1-4 heteroatoms independently selected from nitrogen, oxygen, or sulfur; benzo; or a 5-10 membered heteroaryl having 1-4 heteroatoms independently selected from nitrogen, oxygen, or sulfur. In some embodiments, R² is hydrogen, halogen, C₁₋₆ alkyl, C₃₋₆ cycloalkyl, C₁₋₆ haloalkyl, —OC₁₋₆ alkyl, —OC₃—₆ cycloalkyl, or —OC₁₋₆ haloalkyl.

In some embodiments, R² is hydrogen. In some embodiments, R² is halogen. In some embodiments, R² is C₁₋₆ alkyl. In some embodiments, R² is C₃—₆ cycloalkyl. In some embodiments, R² is C₁₋₆ haloalkyl. In some embodiments, R² is —OC₁₋₆ alkyl. In some embodiments. R² is —OC₃—₆ cycloalkyl. In some embodiments, R² is —OC₁₋₆ haloalkyl.

In some embodiments, R² is methyl. In some embodiments, R² is ethyl. In some embodiments, R² is cyclopropyl.

In some embodiments, an R² and an R¹ group are taken together with their intervening atoms to form an optionally substituted ring selected from a 3-10 membered saturated or partially unsaturated carbocyclyl or heterocyclyl having 1-4 heteroatoms independently selected from nitrogen, oxygen, or sulfur; benzo; or a 5-10 membered heteroaryl having 1-4 heteroatoms independently selected from nitrogen, oxygen, or sulfur.

In some embodiments, an R² and an R¹ group are taken together with their intervening atoms to form an optionally substituted ring selected from a 3-10 membered saturated or partially unsaturated carbocyclyl. In some embodiments, two R¹ groups on the same or adjacent atoms are taken together with their intervening atoms to form an optionally substituted ring selected from a 5-6 membered saturated or partially unsaturated carbocyclyl.

In some embodiments, an R² and an R¹ group are taken together with their intervening atoms to form an optionally substituted ring selected from a 3-10 membered saturated or partially unsaturated heterocyclyl having 1-4 heteroatoms independently selected from nitrogen, oxygen, or sulfur. In some embodiments, an R² and an R¹ group are taken together with their intervening atoms to form an optionally substituted ring selected from a 4-7 membered saturated or partially unsaturated heterocyclyl having 1-4 heteroatoms independently selected from nitrogen, oxygen, or sulfur. In some embodiments, an R² and an R¹ group are taken together with their intervening atoms to form an optionally substituted ring selected from a 5-6 membered saturated or partially unsaturated heterocyclyl having 1-4 heteroatoms independently selected from nitrogen, oxygen, or sulfur.

In some embodiments, an R² and an R¹ group are taken together with their intervening atoms to form an optionally substituted benzo.

In some embodiments, an R² and an R¹ group are taken together with their intervening atoms to form an optionally substituted ring selected from a 5-10 membered heteroaryl having 1-4 heteroatoms independently selected from nitrogen, oxygen, or sulfur. In some embodiments, an R² and an R¹ group are taken together with their intervening atoms to form an optionally substituted ring selected from a 5-6 membered heteroaryl having 1-4 heteroatoms independently selected from nitrogen, oxygen, or sulfur.

In some embodiments, R² is selected from those depicted in the compounds of Table 1 below.

As defined above and described herein, each R^{A} is independently an optionally substituted group selected from C₁₋₆ aliphatic, phenyl, a 3-10 membered saturated or partially unsaturated carbocyclic ring, a 3-10 membered heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, each R^{A} is an optionally substituted C₁₋₆ aliphatic. In some embodiments, each R^{A} is an optionally substituted phenyl. In some embodiments, each R^{A} is an optionally substituted 3-10 membered saturated or partially unsaturated carbocyclic ring. In some embodiments, each R^{A} is an optionally substituted 3-10 membered heterocyclic ring having 1-2 heteroatoms independently selected from nitrogen, oxygen, and sulfur. In some embodiments, each R^{A} is an optionally substituted 5-6 membered heteroaryl ring having 1-4 heteroatoms independently selected from nitrogen, oxygen, and sulfur.

As defined above and described herein, each R^{B} is independently, hydrogen, halogen, —CN, —OR, —SR, —S(O)R, —S(O)₂R, —NR₂, —P(O)(OR)₂, —P(O)(NR₂)OR, —P(O)(NR₂)₂, —Si(OH)₂R, —Si(OH)R₂, —SiR₃, or an optionally substituted C₁₋₄ aliphatic.

In some embodiments, RP is hydrogen. In some embodiments, R^{B} is halogen. In some embodiments, R^{B} is —CN. In some embodiments, R^{B} is —OR. In some embodiments, R^{B} is —SR. In some embodiments, R^{B} is —S(O)R. In some embodiments, R^{B} is —S(O)₂R. In some embodiments, R^{B} is —NR₂In some embodiments, R^{B} is —P(O)(OR)₂. In some embodiments, R^{B} is —P(O)(NR₂)OR. In some embodiments, R^{B} is —P(O)(NR₂)₂. In some embodiments, R^{B} is —Si(OH)₂R. In some embodiments, R^{B} is —Si(OH)R₂. In some embodiments, R^{B} is —SiR₃. In some embodiments, R^{B} is an optionally substituted C—₄ aliphatic.

In some embodiments, R^{B} is C₁₋₄ aliphatic optionally substituted with 1-3 halogens. In some embodiments, R^{B} is C₁₋₄ aliphatic. In some embodiments, R^{B} is methyl. In some embodiments, R^{B} is fluoro.

In some embodiments, R^{B} is selected from those depicted in the compounds of Table 1 below.

As defined above and described herein, L^{1′} is a covalent bond or a C₁₋₃ bivalent hydrocarbon chain wherein 1-2 methylene units of the chain are independently and optionally replaced with —C(O)—, —C(S)—, —CR₂—, —CF₂—, —NR—, —O—, —S—, or —S(O)₂.

In some embodiments, L^{1′} is a covalent bond. In some embodiments, L^{1′} is a C₁₋₃ bivalent hydrocarbon chain wherein 1-2 methylene units of the chain are independently and optionally replaced with —C(O)—, —C(S)—, —CR₂—, —CF₂—, —NR—, —O—, —S—, or —S(O)₂.

In some embodiments, L^{1′} is a covalent bond, —O—, —NR—, —S—, —CR₂—, —NRC(O)—, or —C(O)NR—. In some embodiments, L¹ is —O—, —NR—, —S—, —CR₂—, —NRC(O)—, or —C(O)NR—. In some embodiments, L¹ is —O—. In some embodiments, L¹ is —NR—. In some embodiments, L^{1′} is —S—. In some embodiments, L¹ is —CR₂—. In some embodiments. L¹ is —CH₂—. In some embodiments, L^{1′} is —NRC(O)—. In some embodiments, L¹ is —C(O)NR—. In some embodiments, L^{1′} is —NHC(O)—. In some embodiments. L^{1′} is —C(O)NH—.

In some embodiments, L^{1′} is selected from those depicted in the compounds of Table 1 below.

As defined above and described herein, Ring A is phenylenyl, naphthalenyl, pyridinylenyl, a 4-7 membered saturated or partially unsaturated carbocyclylenyl, a 4-7 membered saturated or partially unsaturated heterocyclylenyl having 1-3 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, an 8-10 membered bicyclic heteroarylenyl having 1-5 heteroatoms independently selected from nitrogen, oxygen, and sulfur, an 8-15 membered saturated or partially unsaturated tricyclic heterocyclylenyl having 1-5 heteroatoms independently selected from nitrogen, oxygen, and sulfur, or an 8-15 membered tricyclic heteroarylenyl having 1-5 heteroatoms independently selected from nitrogen, oxygen, and sulfur.

In some embodiments, Ring A is a phenylenyl. In some embodiments, Ring A is a naphthalenyl. In some embodiments, Ring A is pyridinylenyl. In some embodiments, Ring A is a 4-7 membered saturated or partially unsaturated carbocyclylenyl. In some embodiments, Ring A is a 4-7 membered saturated or partially unsaturated heterocyclylenyl having 1-3 heteroatoms independently selected from nitrogen, oxygen, and sulfur. In some embodiments, Ring A is an 8-10 membered bicyclic saturated or partially unsaturated heterocyclylenyl having 1-2 heteroatoms independently selected from nitrogen, oxygen, and sulfur. In some embodiments, Ring A is a 5-6 membered heteroarylenyl having 1-4 heteroatoms independently selected from nitrogen, oxygen, and sulfur. In some embodiments, Ring A is an 8-10 membered bicyclic heteroarylenyl having 1-5 heteroatoms independently selected from nitrogen, oxygen, and sulfur. In some embodiments, Ring A is an 8-15 membered saturated or partially unsaturated tricyclic heterocyclylenyl having 1-5 heteroatoms independently selected from nitrogen, oxygen, and sulfur. In some embodiments, Ring A is or an 8-15 membered tricyclic heteroarylenyl having 1-5 heteroatoms independently selected from nitrogen, oxygen, and sulfur.

In some embodiments, Ring A is a 9- or 10-membered saturated or partially unsaturated monocyclic or bicyclic heterocyclylenyl or heteroarylenyl containing 1-4 heteroatoms independently selected from nitrogen, oxygen, and sulfur.

In some embodiments, Ring A is an 8-10 membered saturated or partially unsaturated bicyclic heterocyclylenyl or heteroarylenyl containing 1-4 heteroatoms independently selected from nitrogen, oxygen, and sulfur. In some embodiments, Ring A is a 9- or 10-membered saturated or partially unsaturated bicyclic heterocyclylenyl or heteroarylenyl containing 1-4 heteroatoms independently selected from nitrogen, oxygen, and sulfur.

In some embodiments, Ring A is a 9-membered saturated or partially unsaturated bicyclic heterocyclylenyl or heteroarylenyl containing 1-4 heteroatoms independently selected from nitrogen, oxygen, and sulfur. In some embodiments, Ring A is a 9-membered saturated or partially unsaturated bicyclic heterocyclylenyl or heteroarylenyl containing 1 nitrogen and 1 oxygen heteroatom.

In some embodiments, Ring A is a 5,6-fused saturated or partially unsaturated bicyclic heterocyclylenyl or heteroarylenyl containing 1-4 heteroatoms independently selected from nitrogen, oxygen, and sulfur. In some embodiments, Ring A is a 5,6-fused saturated or partially unsaturated bicyclic heterocyclylenyl or heteroarylenyl containing 1 nitrogen and 1 oxygen heteroatom.

In some embodiments, Ring A is not phthalimide.

In some embodiments, Ring A is:

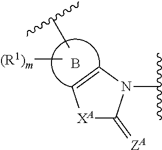

or a pharmaceutically acceptable salt thereof, wherein each R¹, Ring B, X^{A}, and m is as defined above and described herein both individually and in combination; and:
X^{A} is CH₂, CHR¹, C(R¹⁾₂, NH, NR¹, O or S.
Z^{A} is O, S, or NR.

As defined above and described herein, X^{A} is CH₂, CHR¹, C(R′)₂, NH, NR¹, O or S. In some embodiments, X^{A} is CH₂. In some embodiments, X^{A} is CHR¹. In some embodiments, X^{A} is C(R¹)₂, NH, NR¹, O or S.

In some embodiments, X^{A} is C(R¹)₂, wherein each R¹ is optionally substituted C₁₋₆ aliphatic.

As defined above and described herein, Z^{A} is O. S, or NR. In some embodiments, Z^{A} is O. In some embodiments, Z^{A} is S. In some embodiments, Z^{A} is NR.

In some embodiments, Ring A is:

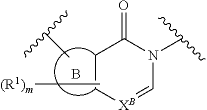

or a pharmaceutically acceptable salt thereof, wherein each R¹, Ring B, and m is as defined above and described herein both individually and in combination, and:
X^{B} is CR² or N.

As defined above and described herein, X^{B} is CR² or N. In some embodiments, X^{B} is CR². In some embodiments, X^{B} is N.

In some embodiments, Ring A is

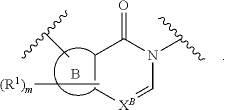

In some embodiments, Ring A is

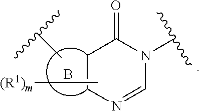

In some embodiments, Ring A is phenylenyl, naphthylenyl, pyridinylenyl,

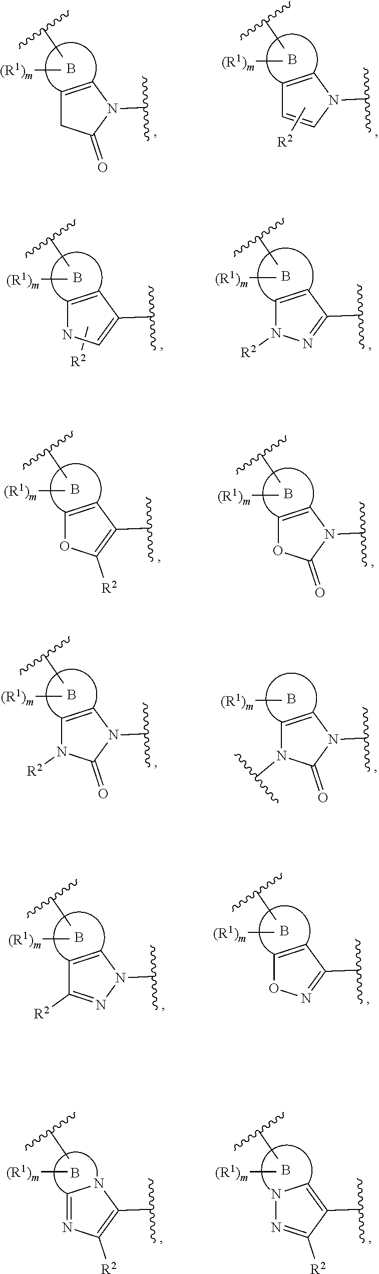

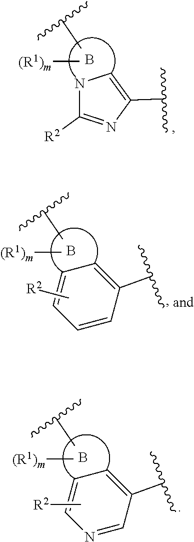

In some embodiments, Ring A is phenylenyl. In some embodiments, Ring A is naphthylenyl. In some embodiments, Ring A is pyridinylenyl. In some embodiments, Ring A is

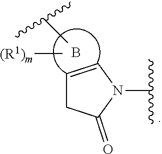

In some embodiments, Ring A is

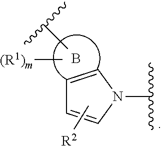

In some embodiments, Ring A is

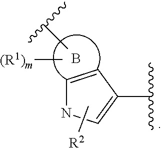

In some embodiments, Ring A is

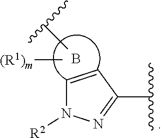

In some embodiments, Ring A is

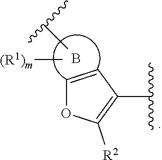

In some embodiments, Ring A is

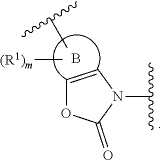

In some embodiments, Ring A is

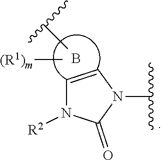

In some embodiments, Ring A is

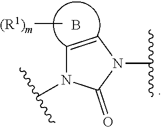

In some embodiments, Ring A is

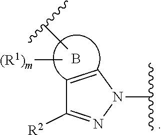

In some embodiments, Ring A is

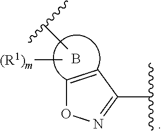

In some embodiments, Ring A is

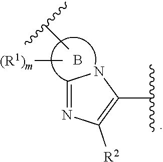

In some embodiments, Ring A is

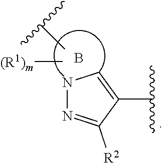

In some embodiments, Ring A is

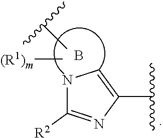

In some embodiments, Ring A is

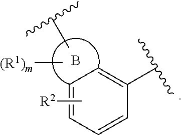

In some embodiments, Ring A is

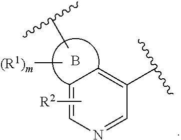

In some embodiments, Ring A is

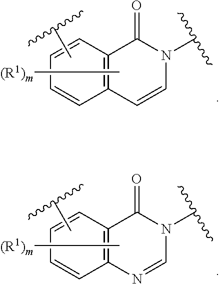

In some embodiments, Ring A is

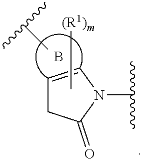

In some embodiments, Ring A is

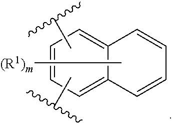

In some embodiments, Ring A is

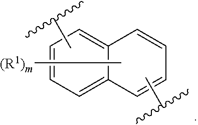

In some embodiments, Ring A is pyridinyl. In some embodiments, Ring A is

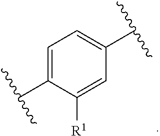

In some embodiments, Ring A is

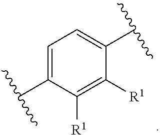

In some embodiments, Ring A is

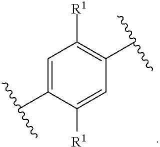

In some embodiments, Ring A is

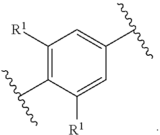

In some embodiments, Ring A is

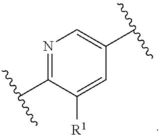

In some embodiments, Ring A is

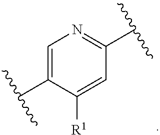

In some embodiments, Ring A is

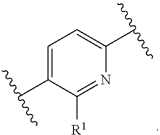

In some embodiments, Ring A is

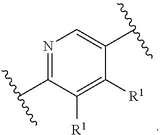

In some embodiments, Ring A is

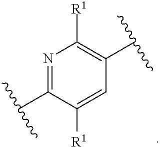

In some embodiments, Ring A is

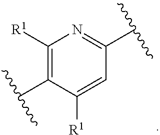

In some embodiments, Ring A is

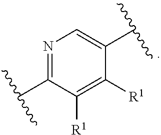

In some embodiments, Ring A is

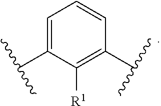

In some embodiments, Ring A is

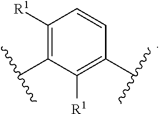

In some embodiments, Ring A is

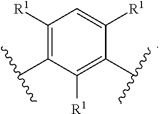

In some embodiments, Ring A is

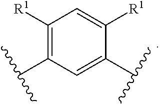

In some embodiments, Ring A is

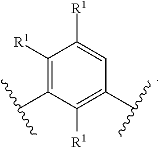

In some embodiments, Ring A is

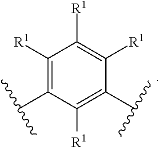

In some embodiments, Ring A is

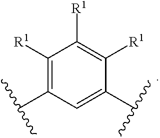

In some embodiments, Ring A is

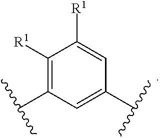

In some embodiments, Ring A is

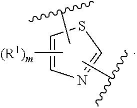

In some embodiments, Ring A is

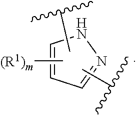

In some embodiments, Ring A is

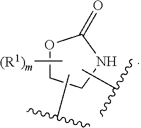

In some embodiments, Ring A is

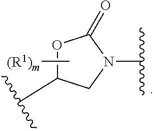

In some embodiments, Ring A is

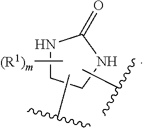

In some embodiments, Ring A is

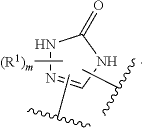

In some embodiments, Ring A is

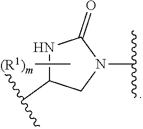

In some embodiments, Ring A is

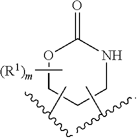

In some embodiments, Ring A is

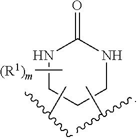

In some embodiments, Ring A is

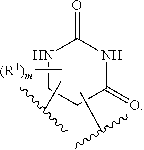

In some embodiments, Ring A is

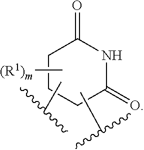

In some embodiments, Ring A is

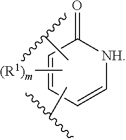

In some embodiments, Ring A is

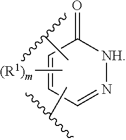

In some embodiments, Ring A is

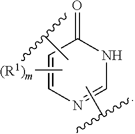

In some embodiments, Ring A is

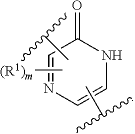

In some embodiments, Ring A is

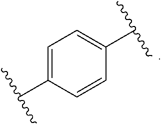 [SMILES: *c1ccc(*)cc1]

In other embodiments Ring A is a 6-10 membered monocyclic or bicyclic heteroaryl containing 1-4 heteroatoms independently selected from nitrogen, oxygen, and sulfur. In other embodiments Ring A is a 9-membered bicyclic heteroaryl containing 1-4 heteroatoms independently selected from nitrogen, oxygen, and sulfur. In some embodiments, Ring A is a 5,6-fused bicyclic heteroarylenyl containing 1-4 heteroatoms independently selected from nitrogen, oxygen, and sulfur.

In other embodiments Ring A is a 9-membered bicyclic heteroaryl containing 1 nitrogen and 1 oxygen heteroatom. In some embodiments, Ring A is a 5,6-fused bicyclic heteroarylenyl containing 1 nitrogen and 1 oxygen heteroatom. In some embodiments, Ring A is benzo[d]oxazolyl, benzo[d]thiazolyl, benzofuran, or benzo[b]thiophenyl. In some embodiments, Ring A is benzo[d]oxazolyl. In some embodiments, Ring A is benzo[d]thiazolyl. In some embodiments, Ring A is benzofuran. In some embodiments, Ring A is benzo[b]thiophenyl.

In other embodiments Ring A is a 9-membered bicyclic heteroaryl containing 1-3 nitrogen heteroatoms. In some embodiments, Ring A is a 5,6-fused bicyclic heteroarylenyl containing 1-3 nitrogen heteroatoms. In some embodiments, Ring A is indolyl, azaindolyl (e.g., 4-, 5-, 6-, or 7-azaindolyl), indazolyl, or azaindazolyl (e.g., 4-, 5-, 6-, or 7-azaindazolyl). In some embodiments, Ring A is indolyl. In some embodiments, Ring A is azaindolyl (e.g., 4-, 5-, 6-, or 7-azaindolyl). In some embodiments, Ring A is indazolyl. In some embodiments, Ring A is azaindazolyl (e.g., 4-, 5-, 6-, or 7-azaindazolyl).

In other embodiments Ring A is a 10-membered bicyclic heteroaryl containing 1-4 heteroatoms independently selected from nitrogen, oxygen, and sulfur. In some embodiments, Ring A is a 6,6-fused bicyclic heteroarylenyl containing 1-4 heteroatoms independently selected from nitrogen, oxygen, and sulfur. In some embodiments, Ring A is a 6,6-fused bicyclic heteroarylenyl containing 1 nitrogen heteroatom.

In some embodiments, Ring A is quinolinyl or isoquinolinyl. In some embodiments, Ring A is quinolinyl. In some embodiments, Ring A is isoquinolinyl.

In other embodiments Ring A is a 6-10 membered monocyclic or bicyclic saturated or partially unsaturated heterocyclylenyl containing 1-4 heteroatoms independently selected from nitrogen, oxygen, and sulfur. In other embodiments Ring A is a 9-membered bicyclic saturated or partially unsaturated heterocyclylenyl containing 1-4 heteroatoms independently selected from nitrogen, oxygen, and sulfur. In other embodiments Ring A is a 9-membered bicyclic saturated or partially unsaturated heterocyclylenyl containing 1 nitrogen heteroatom. In some embodiments, Ring A is a 5,6-fused bicyclic saturated or partially unsaturated heterocyclylenyl containing 1-4 heteroatoms independently selected from nitrogen, oxygen, and sulfur. In some embodiments, Ring A is a 5,6-fused bicyclic saturated or partially unsaturated heterocyclylenyl containing 1 nitrogen heteroatom.

In other embodiments Ring A is a 6-10 membered monocyclic or bicyclic saturated or partially unsaturated heterocyclylenyl containing 1-4 heteroatoms independently selected from nitrogen, oxygen, and sulfur, and having an oxo group. In other embodiments Ring A is a 9-membered bicyclic saturated or partially unsaturated heterocyclylenyl containing 1-4 heteroatoms independently selected from nitrogen, oxygen, and sulfur, and having an oxo group. In other embodiments Ring A is a 9-membered bicyclic saturated or partially unsaturated heterocyclylenyl containing 1 nitrogen heteroatom, and having an oxo group. In some embodiments, Ring A is a 5,6-fused bicyclic saturated or partially unsaturated heterocyclylenyl containing 1-4 heteroatoms independently selected from nitrogen, oxygen, and sulfur, and having an oxo group. In some embodiments, Ring A is a 5,6-fused bicyclic saturated or partially unsaturated heterocyclylenyl containing 1 nitrogen heteroatom, and having an oxo group.

In some embodiments, Ring A is indolinyl, indolinonyl, isoindolinyl, isoindolinonyl, isoindolinedionyl, pyrrolopyridinyl, or pyrrolopyrimidinyl. In some embodiments, Ring A is indolinyl. In some embodiments, Ring A is indolinonyl. In some embodiments, Ring A is isoindolinyl. In some embodiments, Ring A is isoindolinonyl. In some embodiments, Ring A is isoindolinedionyl. In some embodiments, Ring A is pyrrolopyridinyl. In some embodiments, Ring A is pyrrolopyrimidinyl.

In some embodiments, Ring A is benzoxazolyl, benzothiazolyl, indazolyl, or azaindazolyl. In some embodiments, Ring A is benzoxazolyl. In some embodiments, Ring A is benzothiazolyl. In some embodiments, Ring A is indazolyl. In some embodiments, Ring A is azaindazolyl (e.g., 4-, 5-, 6-, or 7-azaindazolyl).

In some embodiments, Ring A is 2,3-dihydrobenzofuranyl, indolinyl, or 2,3-dihydrobenzothiophenyl. In some embodiments, Ring A is 2,3-dihydrobenzofuranyl. In some embodiments, Ring A is indolinyl. In some embodiments, Ring A is 2,3-dihydrobenzothiophenyl.

In some embodiments, Ring A is

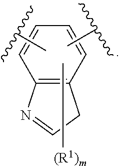

In some embodiments, Ring A is

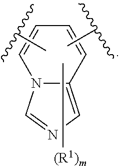

In some embodiments, Ring A is

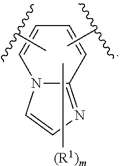

In some embodiments, Ring A is

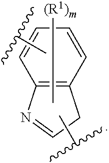

In some embodiments, Ring A is

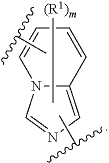

In some embodiments, Ring A is

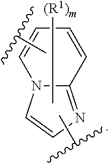

In some embodiments, Ring A is

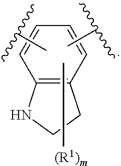

In some embodiments, Ring A is

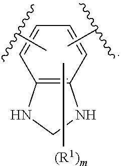

In some embodiments Ring A is

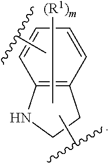

In some embodiments, Ring A is

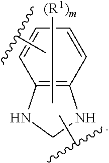

In some embodiments, Ring A is

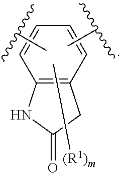

In some embodiments, Ring A is

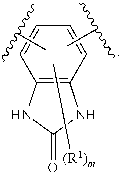

In some embodiments, Ring A is

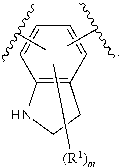

In some embodiments, Ring A is

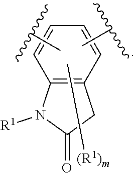

In some embodiments, Ring A is

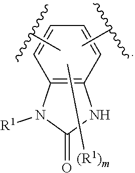

In some embodiments, Ring A is

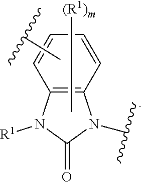

In some embodiments, Ring A is

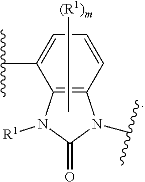

In some embodiments, Ring A is

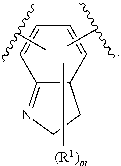

In some embodiments, Ring A is

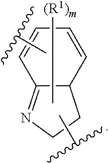

In some embodiments, Ring A is

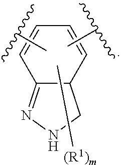

In some embodiments, Ring A is

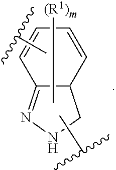

In some embodiments, Ring A is

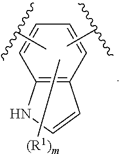

In some embodiments, Ring A is

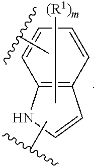

In some embodiments, Ring A is

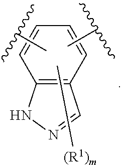

In some embodiments, Ring A is

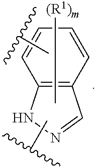

In some embodiments, Ring A is

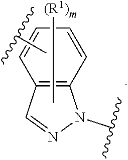

In some embodiments, Ring A is

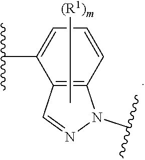

In some embodiments, Ring A is

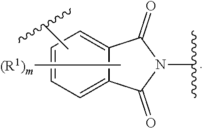

In some embodiments, Ring A is

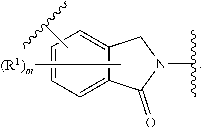

In some embodiments, Ring A is

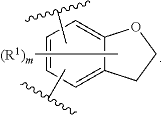

In some embodiments, Ring A is

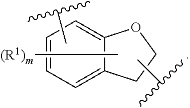

In some embodiments, Ring A is

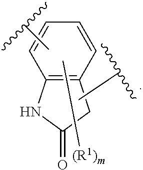

In some embodiments, Ring A is an 10-15 membered saturated or partially unsaturated tricyclic heterocyclylenyl having 1-5 heteroatoms independently selected from nitrogen, oxygen, and sulfur.

In some embodiments, Ring A is a 12-membered saturated or partially unsaturated tricyclic heterocyclylenyl having 1-5 heteroatoms independently selected from nitrogen, oxygen, and sulfur. In some embodiments, Ring A is a 12-membered saturated or partially unsaturated tricyclic heterocyclylenyl having 1-5 nitrogen heteroatoms. In some embodiments, Ring A is a 12-membered saturated or partially unsaturated tricyclic heterocyclylenyl having 3 nitrogen heteroatoms.

In some embodiments, Ring A is a 5,6,5-fused saturated or partially unsaturated tricyclic heterocyclylenyl having 1-5 heteroatoms independently selected from nitrogen, oxygen, and sulfur. In some embodiments, Ring A is a 5,6,5-fused membered saturated or partially unsaturated tricyclic heterocyclylenyl having 1-5 nitrogen heteroatoms. In some embodiments, Ring A is a 5,6,5-fused membered saturated or partially unsaturated tricyclic heterocyclylenyl having 3 nitrogen heteroatoms.

In some embodiments, Ring A is a 5,5,6-fused saturated or partially unsaturated tricyclic heterocyclylenyl having 1-5 heteroatoms independently selected from nitrogen, oxygen, and sulfur. In some embodiments, Ring A is a 5,5,6-fused membered saturated or partially unsaturated tricyclic heterocyclylenyl having 1-5 nitrogen heteroatoms. In some embodiments, Ring A is a 5,5,6-fused membered saturated or partially unsaturated tricyclic heterocyclylenyl having 3 nitrogen heteroatoms.

In some embodiments, Ring A is a 13-membered saturated or partially unsaturated tricyclic heterocyclylenyl having 1-5 heteroatoms independently selected from nitrogen, oxygen, and sulfur. In some embodiments, Ring A is a 13-membered saturated or partially unsaturated tricyclic heterocyclylenyl having 1-5 nitrogen heteroatoms. In some embodiments, Ring A is a 13-membered saturated or partially unsaturated tricyclic heterocyclylenyl having 3 nitrogen heteroatoms.

In some embodiments, Ring A is a 5,6,6-fused saturated or partially unsaturated tricyclic heterocyclylenyl having 1-5 heteroatoms independently selected from nitrogen, oxygen, and sulfur. In some embodiments, Ring A is a 5,6,6-fused membered saturated or partially unsaturated tricyclic heterocyclylenyl having 1-5 nitrogen heteroatoms. In some embodiments, Ring A is a 5,6,6-fused membered saturated or partially unsaturated tricyclic heterocyclylenyl having 3 nitrogen heteroatoms.

In some embodiments, Ring A is a 6,5,6-fused saturated or partially unsaturated tricyclic heterocyclylenyl having 1-5 heteroatoms independently selected from nitrogen, oxygen, and sulfur. In some embodiments, Ring A is a 6,5,6-fused membered saturated or partially unsaturated tricyclic heterocyclylenyl having 1-5 nitrogen heteroatoms. In some embodiments, Ring A is a 6,5,6-fused membered saturated or partially unsaturated tricyclic heterocyclylenyl having 3 nitrogen heteroatoms.

In some embodiments, Ring A is a 14-membered saturated or partially unsaturated tricyclic heterocyclylenyl having 1-5 heteroatoms independently selected from nitrogen, oxygen, and sulfur.

In some embodiments, Ring A is or an 10-15 membered tricyclic heteroarylenyl having 1-5 heteroatoms independently selected from nitrogen, oxygen, and sulfur.

In some embodiments, Ring A is a 12-membered saturated or partially unsaturated tricyclic heteroarylenyl having 1-5 heteroatoms independently selected from nitrogen, oxygen, and sulfur. In some embodiments, Ring A is a 12-membered saturated or partially unsaturated tricyclic heteroarylenyl having 1-5 nitrogen heteroatoms. In some embodiments, Ring A is a 12-membered saturated or partially unsaturated tricyclic heteroarylenyl having 3 nitrogen heteroatoms.

In some embodiments, Ring A is a 5,6,5-fused saturated or partially unsaturated tricyclic heteroarylenyl having 1-5 heteroatoms independently selected from nitrogen, oxygen, and sulfur. In some embodiments, Ring A is a 5,6,5-fused membered saturated or partially unsaturated tricyclic heteroarylenyl having 1-5 nitrogen heteroatoms. In some embodiments, Ring A is a 5,6,5-fused membered saturated or partially unsaturated tricyclic heteroarylenyl having 3 nitrogen heteroatoms.

In some embodiments, Ring A is a 5,5,6-fused saturated or partially unsaturated tricyclic heteroarylenyl having 1-5 heteroatoms independently selected from nitrogen, oxygen, and sulfur. In some embodiments, Ring A is a 5,5,6-fused membered saturated or partially unsaturated tricyclic heteroarylenyl having 1-5 nitrogen heteroatoms. In some embodiments, Ring A is a 5,5,6-fused membered saturated or partially unsaturated tricyclic heteroarylenyl having 3 nitrogen heteroatoms.

In some embodiments, Ring A is a 13-membered saturated or partially unsaturated tricyclic heteroarylenyl having 1-5 heteroatoms independently selected from nitrogen, oxygen, and sulfur. In some embodiments, Ring A is a 13-membered saturated or partially unsaturated tricyclic heteroarylenyl having 1-5 nitrogen heteroatoms. In some embodiments, Ring A is a 13-membered saturated or partially unsaturated tricyclic heteroarylenyl having 3 nitrogen heteroatoms.

In some embodiments, Ring A is a 5,6,6-fused saturated or partially unsaturated tricyclic heteroarylenyl having 1-5 heteroatoms independently selected from nitrogen, oxygen, and sulfur. In some embodiments, Ring A is a 5,6,6-fused membered saturated or partially unsaturated tricyclic heteroarylenyl having 1-5 nitrogen heteroatoms. In some embodiments, Ring A is a 5,6,6-fused membered saturated or partially unsaturated tricyclic heteroarylenyl having 3 nitrogen heteroatoms.

In some embodiments, Ring A is a 6,5,6-fused saturated or partially unsaturated tricyclic heteroarylenyl having 1-5 heteroatoms independently selected from nitrogen, oxygen, and sulfur. In some embodiments, Ring A is a 6,5,6-fused membered saturated or partially unsaturated tricyclic heteroarylenyl having 1-5 nitrogen heteroatoms. In some embodiments, Ring A is a 6,5,6-fused membered saturated or partially unsaturated tricyclic heteroarylenyl having 3 nitrogen heteroatoms.

In some embodiments, Ring A is a 14-membered saturated or partially unsaturated tricyclic heteroarylenyl having 1-5 heteroatoms independently selected from nitrogen, oxygen, and sulfur.

In some embodiments, Ring A is of formula ABC-1 or ABC-2:

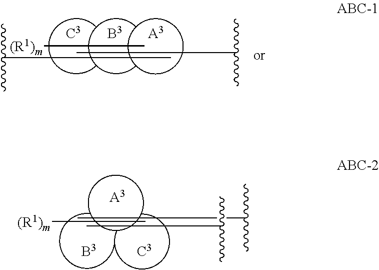

or a pharmaceutically acceptable salt thereof, wherein each R¹ and m is as defined above and described herein both individually and in combination; and:
each of Ring A³, Ring B³, and Ring C³ is independently a fused ring selected from a 3-7 membered saturated or partially unsaturated carbocyclylenyl: phenyl; a 3-7 membered saturated or partially unsaturated heterocyclylenyl having 1-5 heteroatoms independently selected from nitrogen, oxygen, and sulfur; or a 5-6 membered heteroarylenyl having 1-5 heteroatoms independently selected from nitrogen, oxygen, and sulfur.

As defined above and described herein, each of Ring A³, Ring B³, and Ring C³ is independently a fused ring selected from a 3-7 membered saturated or partially unsaturated carbocyclylenyl; phenyl; a 3-7 membered saturated or partially unsaturated heterocyclylenyl having 1-5 heteroatoms independently selected from nitrogen, oxygen, and sulfur; or a 5-6 membered heteroarylenyl having 1-5 heteroatoms independently selected from nitrogen, oxygen, and sulfur.

In some embodiments, Ring A³ is a fused 3-7 membered saturated or partially unsaturated carbocyclylenyl. In some embodiments, Ring A³ is a fused 4-7 membered saturated or partially unsaturated carbocyclylenyl. In some embodiments, Ring A³ is a fused 5-membered saturated or partially unsaturated carbocyclylenyl. In some embodiments, Ring A³ is a fused 6-membered saturated or partially unsaturated carbocyclylenyl. In some embodiments, Ring A³ is fused phenyl.

In some embodiments, Ring A³ is a fused 3-7 membered saturated or partially unsaturated heterocyclylenyl having 1-5 heteroatoms independently selected from nitrogen, oxygen, and sulfur. In some embodiments, Ring A³ is a fused 4-7 membered saturated or partially unsaturated heterocyclylenyl having 1-5 heteroatoms independently selected from nitrogen, oxygen, and sulfur. In some embodiments, Ring A³ is a fused 5-membered saturated or partially unsaturated heterocyclylenyl having 1-5 heteroatoms independently selected from nitrogen, oxygen, and sulfur. In some embodiments, Ring A³ is a fused 5-membered saturated or partially unsaturated heterocyclylenyl having 1-2 nitrogen heteroatoms. In some embodiments, Ring A³ is a fused 5-membered saturated or partially unsaturated heterocyclylenyl having 1-5 heteroatoms independently selected from nitrogen, oxygen, and sulfur, and an oxo group. In some embodiments, Ring A³ is a fused 5-membered saturated or partially unsaturated heterocyclylenyl having 1-2 nitrogen heteroatoms, and an oxo group

In some embodiments, Ring A³ is a fused 6-membered saturated or partially unsaturated heterocyclylenyl having 1-5 heteroatoms independently selected from nitrogen, oxygen, and sulfur. In some embodiments, Ring A³ is a fused 6-membered saturated or partially unsaturated heterocyclylenyl having 1-2 nitrogen heteroatoms. In some embodiments, Ring A³ is a fused 6-membered saturated or partially unsaturated heterocyclylenyl having 1-5 heteroatoms independently selected from nitrogen, oxygen, and sulfur, and an oxo group. In some embodiments, Ring A³ is a fused 6-membered saturated or partially unsaturated heterocyclylenyl having 1-2 nitrogen heteroatoms, and an oxo group

In some embodiments, Ring A³ is a fused 5-6 membered heteroarylenyl having 1-5 heteroatoms independently selected from nitrogen, oxygen, and sulfur. In some embodiments, Ring A³ is a fused 5-membered heteroarylenyl having 1-5 heteroatoms independently selected from nitrogen, oxygen, and sulfur. In some embodiments, Ring A³ is a fused 6-membered heteroarylenyl having 1-5 heteroatoms independently selected from nitrogen, oxygen, and sulfur.

In some embodiments, Ring B³ is a fused 3-7 membered saturated or partially unsaturated carbocyclylenyl. In some embodiments, Ring B³ is a fused 4-7 membered saturated or partially unsaturated carbocyclylenyl. In some embodiments, Ring B³ is a fused 5-membered saturated or partially unsaturated carbocyclylenyl. In some embodiments, Ring B³ is a fused 6-membered saturated or partially unsaturated carbocyclylenyl. In some embodiments, Ring B³ is fused phenyl.

In some embodiments, Ring B³ is a fused 3-7 membered saturated or partially unsaturated heterocyclylenyl having 1-5 heteroatoms independently selected from nitrogen, oxygen, and sulfur. In some embodiments, Ring B³ is a fused 4-7 membered saturated or partially unsaturated heterocyclylenyl having 1-5 heteroatoms independently selected from nitrogen, oxygen, and sulfur. In some embodiments, Ring B³ is a fused 5-membered saturated or partially unsaturated heterocyclylenyl having 1-5 heteroatoms independently selected from nitrogen, oxygen, and sulfur. In some embodiments, Ring B³ is a fused 5-membered saturated or partially unsaturated heterocyclylenyl having 1-2 nitrogen heteroatoms. In some embodiments, Ring B³ is a fused 5-membered saturated or partially unsaturated heterocyclylenyl having 1-5 heteroatoms independently selected from nitrogen, oxygen, and sulfur, and an oxo group. In some embodiments, Ring B³ is a fused 5-membered saturated or partially unsaturated heterocyclylenyl having 1-2 nitrogen heteroatoms, and an oxo group

In some embodiments, Ring B³ is a fused 6-membered saturated or partially unsaturated heterocyclylenyl having 1-5 heteroatoms independently selected from nitrogen, oxygen, and sulfur. In some embodiments, Ring B³ is a fused 6-membered saturated or partially unsaturated heterocyclylenyl having 1-2 nitrogen heteroatoms. In some embodiments, Ring B³ is a fused 6-membered saturated or partially unsaturated heterocyclylenyl having 1-5 heteroatoms independently selected from nitrogen, oxygen, and sulfur, and an oxo group. In some embodiments, Ring B³ is a fused 6-membered saturated or partially unsaturated heterocyclylenyl having 1-2 nitrogen heteroatoms, and an oxo group

In some embodiments, Ring B³ is a fused 5-6 membered heteroarylenyl having 1-5 heteroatoms independently selected from nitrogen, oxygen, and sulfur. In some embodiments, Ring B³ is a fused 5-membered heteroarylenyl having 1-5 heteroatoms independently selected from nitrogen, oxygen, and sulfur. In some embodiments, Ring B³ is a fused 6-membered heteroarylenyl having 1-5 heteroatoms independently selected from nitrogen, oxygen, and sulfur.

In some embodiments, Ring C³ is a fused 3-7 membered saturated or partially unsaturated carbocyclylenyl. In some embodiments, Ring C³ is a fused 4-7 membered saturated or partially unsaturated carbocyclylenyl. In some embodiments, Ring C³ is a fused 5-membered saturated or partially unsaturated carbocyclylenyl. In some embodiments, Ring C³ is a fused 6-membered saturated or partially unsaturated carbocyclylenyl. In some embodiments, Ring C³ is fused phenyl.

In some embodiments, Ring C³ is a fused 3-7 membered saturated or partially unsaturated heterocyclylenyl having 1-5 heteroatoms independently selected from nitrogen, oxygen, and sulfur. In some embodiments, Ring C³ is a fused 4-7 membered saturated or partially unsaturated heterocyclylenyl having 1-5 heteroatoms independently selected from nitrogen, oxygen, and sulfur. In some embodiments, Ring C³ is a fused 5-membered saturated or partially unsaturated heterocyclylenyl having 1-5 heteroatoms independently selected from nitrogen, oxygen, and sulfur. In some embodiments, Ring C³ is a fused 5-membered saturated or partially unsaturated heterocyclylenyl having 1-2 nitrogen heteroatoms. In some embodiments, Ring C³ is a fused 5-membered saturated or partially unsaturated heterocyclylenyl having 1-5 heteroatoms independently selected from nitrogen, oxygen, and sulfur, and an oxo group. In some embodiments, Ring C³ is a fused 5-membered saturated or partially unsaturated heterocyclylenyl having 1-2 nitrogen heteroatoms, and an oxo group

In some embodiments, Ring C³ is a fused 6-membered saturated or partially unsaturated heterocyclylenyl having 1-5 heteroatoms independently selected from nitrogen, oxygen, and sulfur. In some embodiments, Ring C³ is a fused 6-membered saturated or partially unsaturated heterocyclylenyl having 1-2 nitrogen heteroatoms. In some embodiments, Ring C³ is a fused 6-membered saturated or partially unsaturated heterocyclylenyl having 1-5 heteroatoms independently selected from nitrogen, oxygen, and sulfur, and an oxo group. In some embodiments, Ring C³ is a fused 6-membered saturated or partially unsaturated heterocyclylenyl having 1-2 nitrogen heteroatoms, and an oxo group.

In some embodiments, Ring C³ is a fused 5-6 membered heteroarylenyl having 1-5 heteroatoms independently selected from nitrogen, oxygen, and sulfur. In some embodiments, Ring C³ is a fused 5-membered heteroarylenyl having 1-5 heteroatoms independently selected from nitrogen, oxygen, and sulfur. In some embodiments, Ring C³ is a fused 6-membered heteroarylenyl having 1-5 heteroatoms independently selected from nitrogen, oxygen, and sulfur.

In some embodiments, Ring A is of formula ABC-1 having a structure of formulae ABC-1a, ABC-1b, or ABC-1c:

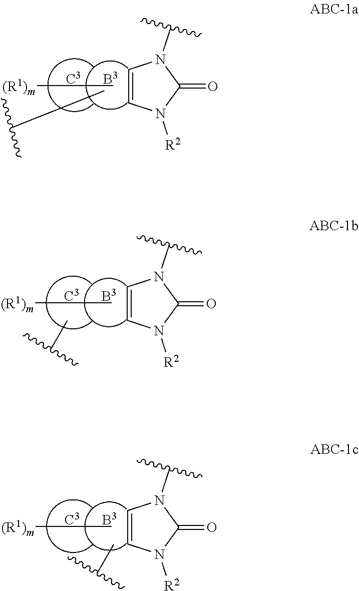

or a pharmaceutically acceptable salt thereof.

In some embodiments, Ring A is of formula ABC-1 having a structure of formulae ABC-1d, ABC-1e, or ABC-1f:

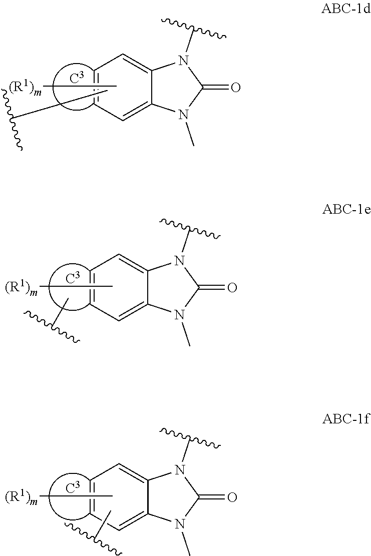

or a pharmaceutically acceptable salt thereof.

In some embodiments, Ring A is of formula ABC-1 having a structure of formulae ABC-1g, ABC-1h, ABC-1i, ABC-1j, ABC-1k, ABC-1l, ABC-1m, or ABC-1n:

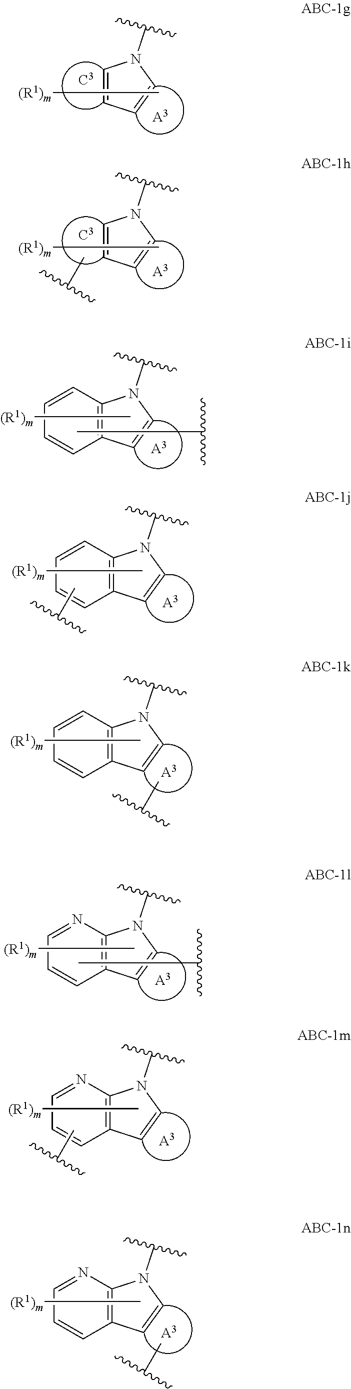

or a pharmaceutically acceptable salt thereof.

In some embodiments, Ring A is of formula ABC-2 having a structure of formulae ABC-2a, ABC-2b, ABC-2c, ABC-2d, ABC-2e, ABC-2f, or ABC-2g:

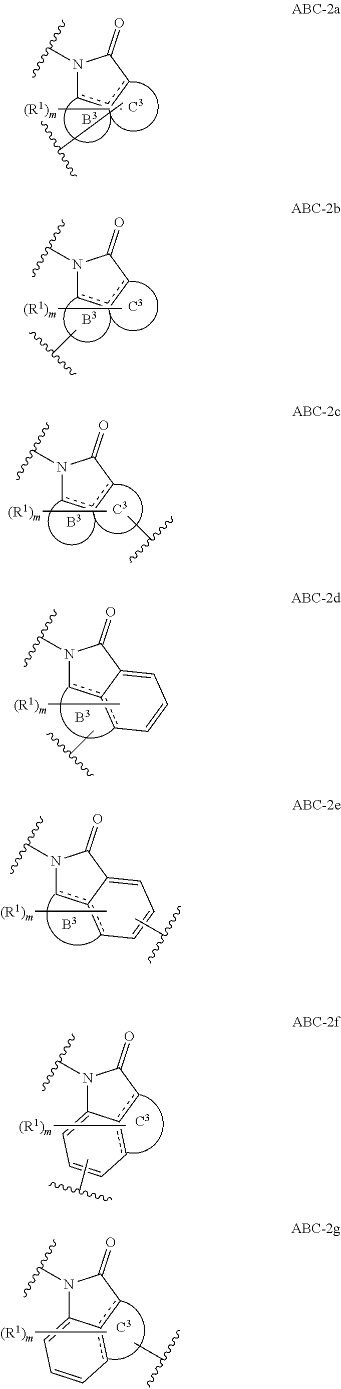

or a pharmaceutically acceptable salt thereof.

In some embodiments, Ring A is

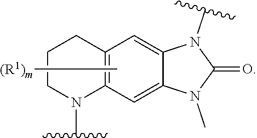

In some embodiments, Ring A is

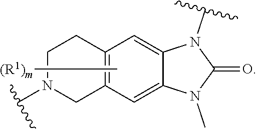

In some embodiments, Ring A is

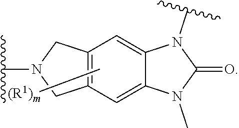

In some embodiments, Ring A is

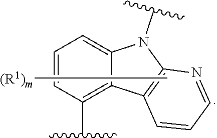

In some embodiments, Ring A is

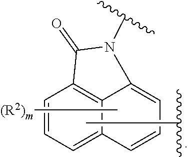

In some embodiments, Ring A is

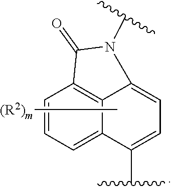

In some embodiments, Ring A and its R¹ substituents is

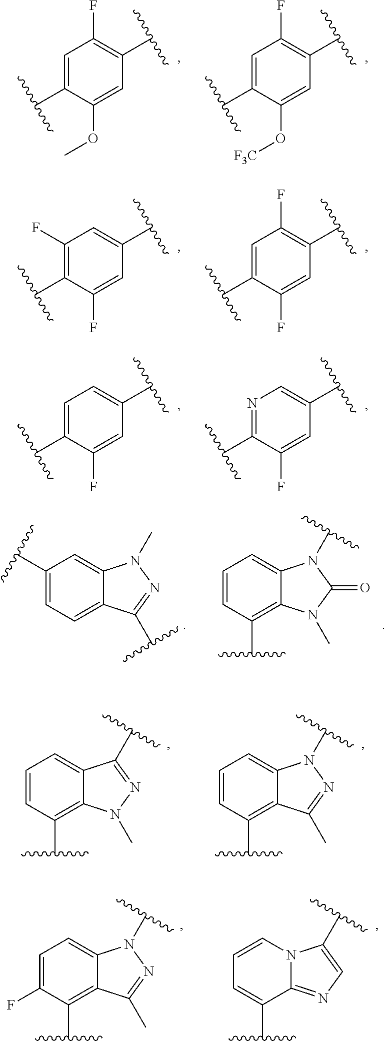

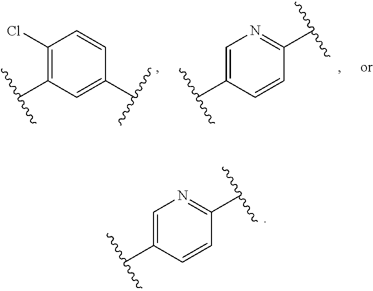 [SMILES: *c1ccc(Cl)c(*)c1 ; *c1ccc(*)nc1 ; *c1ccc(*)nc1]

In some embodiments, Ring A, with its R¹ substituents and L¹ linker, is

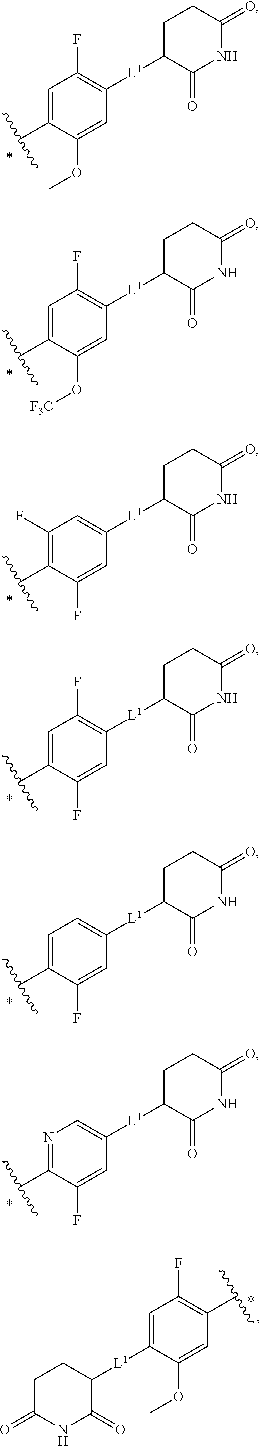

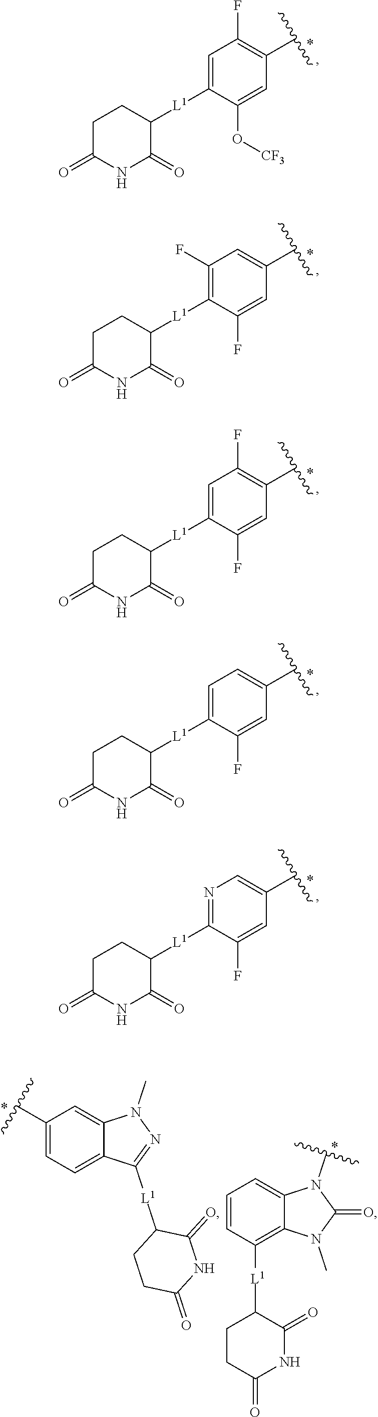

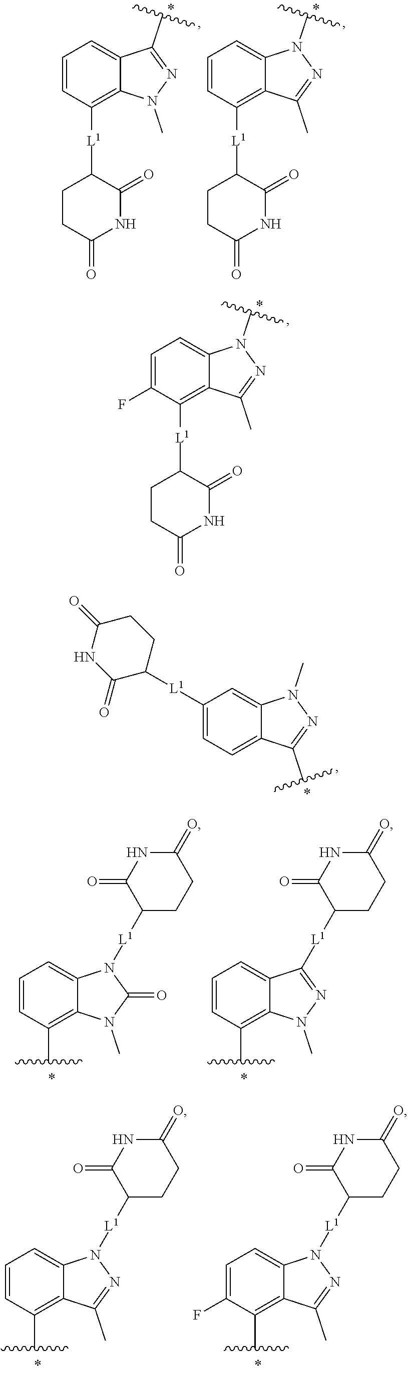

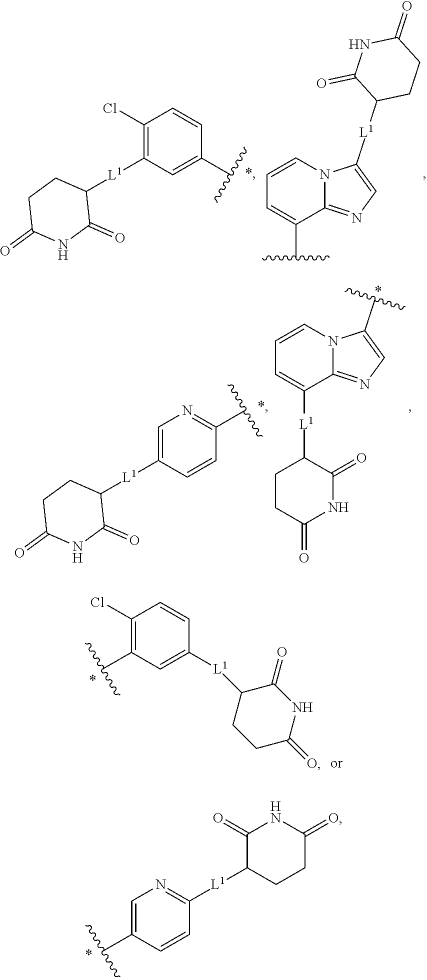

wherein

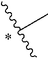

is the connection point to linker L.

In some embodiments, Ring A is selected from those depicted in the compounds of Table 1 below.

As defined above and described herein, Ring B is a fused ring selected from benzo, a saturated or partially unsaturated 4-7 membered carbocyclyl, a saturated or partially unsaturated 4-7 membered heterocyclyl having 1-2 heteroatoms independently selected from nitrogen, oxygen, and sulfur, and a 5-6 membered heteroaryl containing 1-4 heteroatoms independently selected from nitrogen, oxygen, and sulfur.

In some embodiments, Ring B is a fused ring selected from benzo or a 5-6 membered heteroaryl containing 1-4 heteroatoms independently selected from nitrogen, oxygen, and sulfur.

In some embodiments, Ring B is benzo. In some embodiments, Ring B is a 5-6 membered heteroaryl containing 1-4 heteroatoms independently selected from nitrogen, oxygen, and sulfur. In some embodiments, Ring B is pyridinyl.

In some embodiments, Ring B is a fused saturated or partially unsaturated 4-7 membered carbocyclyl. In some embodiments, Ring B is a fused saturated or partially unsaturated 4-6 membered carbocyclyl. In some embodiments, Ring B is a fused saturated or partially unsaturated 5-6 membered carbocyclyl. In some embodiments, Ring B is a saturated or partially unsaturated 4-7 membered heterocyclyl having 1-2 heteroatoms independently selected from nitrogen, oxygen, and sulfur. In some embodiments, Ring B is a saturated or partially unsaturated 4-6 membered heterocyclyl having 1-2 heteroatoms independently selected from nitrogen, oxygen, and sulfur. In some embodiments, Ring B is a saturated or partially unsaturated 5-6 membered heterocyclyl having 1-2 heteroatoms independently selected from nitrogen, oxygen, and sulfur.

In some embodiments, Ring B is selected from those depicted in the compounds of Table 1 below.

In some embodiments, Ring A and Ring B are

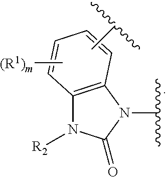

In some embodiments, Ring A and Ring B are

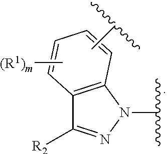

In some embodiments, Ring A and Ring B are

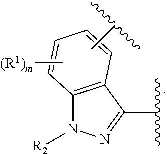

In some embodiments, Ring A and Ring B are

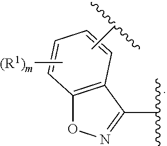

In some embodiments, Ring A and Ring B are

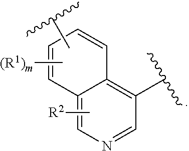

In some embodiments, LBM is

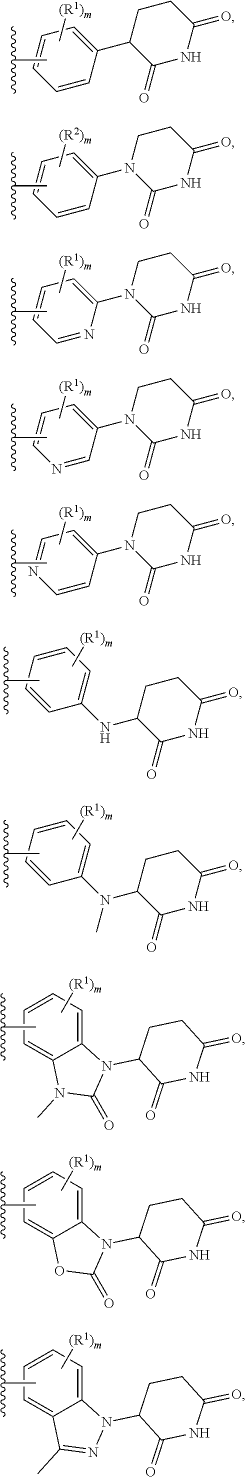

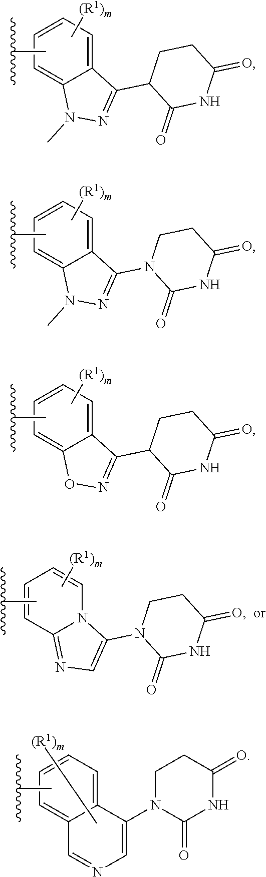

In some embodiments, LBM is

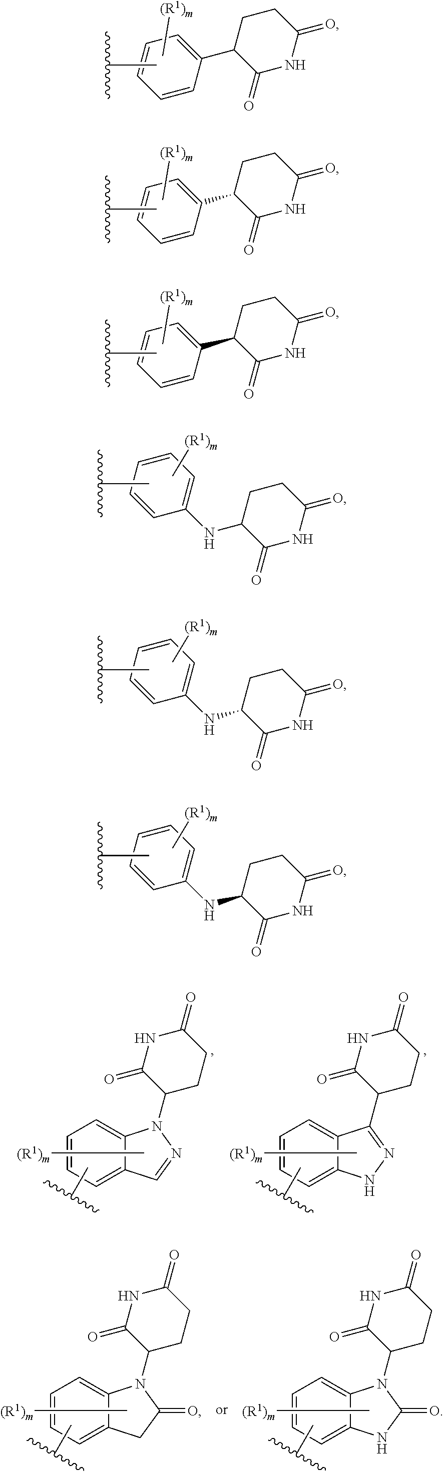

As defined above and described herein, R is independently hydrogen or an optionally substituted group selected from C₁₋₆ aliphatic, phenyl, a 3-7 membered saturated or partially unsaturated carbocyclic ring, a 3-7 membered heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur; or two R groups on the same or adjacent atoms or R^{B} and an R group are taken together with their intervening atoms to form an optionally substituted 3-11 membered saturated or partially unsaturated monocyclic, bicyclic, bridged bicyclic, or spirocyclic carbocyclic or heterocyclic ring having 0-3 heteroatoms independently selected from nitrogen, oxygen, and sulfur; or two R groups on the same carbon or nitrogen are taken together with their intervening atoms to form an optionally substituted 4-11 membered saturated or partially unsaturated monocyclic, bicyclic, bridged bicyclic, or spirocyclic carbocyclic or heterocyclic ring having 0-3 heteroatoms, in addition to the carbon or nitrogen from which the two R groups are attached, independently selected from nitrogen, oxygen, and sulfur.

In some embodiments, R is hydrogen. In some embodiments, R is an optionally substituted C₁₋₆ aliphatic. In some embodiments, R is an optionally substituted phenyl. In some embodiments. R is an optionally substituted 3-7 membered saturated or partially unsaturated carbocyclic ring. In some embodiments. R is an optionally substituted 3-7 membered heterocyclic ring having 1-2 heteroatoms independently selected from nitrogen, oxygen, and sulfur. In some embodiments, R is an optionally substituted 5-6 membered heteroaryl ring having 1-4 heteroatoms independently selected from nitrogen, oxygen, and sulfur. In some embodiments, two R groups on the same or adjacent atoms or R^{B} and an R group are taken together with their intervening atoms to form an optionally substituted 3-11 membered saturated or partially unsaturated monocyclic, bicyclic, bridged bicyclic, or spirocyclic carbocyclic or heterocyclic ring having 0-3 heteroatoms independently selected from nitrogen, oxygen, and sulfur. In some embodiments, two R groups on the same carbon or nitrogen are taken together with their intervening atoms to form a 4-11 membered saturated or partially unsaturated monocyclic, bicyclic, bridged bicyclic, or spirocyclic carbocyclic or heterocyclic ring having 0-3 heteroatoms, in addition to the carbon or nitrogen from which the two R groups are attached, independently selected from nitrogen, oxygen, and sulfur. In some embodiments, two R groups on the same carbon or nitrogen are taken together with their intervening atoms to form an optionally substituted 4-11 membered saturated or partially unsaturated monocyclic, bicyclic, bridged bicyclic, or spirocyclic carbocyclic. In some embodiments, two R groups on the same carbon or nitrogen are optionally taken together with their intervening atoms to form an optionally substituted heterocyclic ring having 0-3 heteroatoms, in addition to the carbon or nitrogen from which the two R groups are attached, independently selected from nitrogen, oxygen, and sulfur.

In some embodiments, R is C₁₋₆ alkyl (e.g., methyl, ethyl, isopropyl, etc.). In some embodiments, R is C₁₋₆ haloalkyl (e.g., —CF₃, CHF₂, etc.).

In some embodiments, R is selected from those depicted in the compounds of Table 1 below.

As defined above and described herein, m is 0, 1, 2, 3, 4, or 5. In some embodiments, m is 0. In some embodiments, m is 1. In some embodiments, m is 2. In some embodiments, m is 3. In some embodiments, m is 4. In some embodiments, m is 5. In some embodiments, m is 0 or 1. In some embodiments, m is 1 or 2. In some embodiments, m is 0, 1, or 2.

In some embodiments, m is selected from those depicted in the compounds of Table 1 below.

In some embodiments, LBM is a non-IMiD (immune modulatory drug), e.g., not thalidomide or a derivative thereof wherein Ring A is phthalimide.

In some embodiments, LBM is

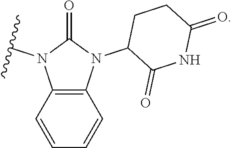 [SMILES: *n1c(=O)n(C2CCC(=O)NC2=O)c2ccccc21]

In some embodiments, LBM is

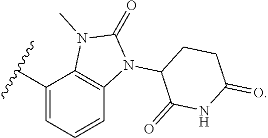 [SMILES: *c1cccc2c1n(C)c(=O)n2C1CCC(=O)NC1=O]

In some embodiments, LBM is

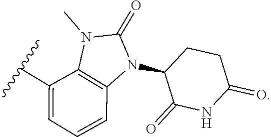 [SMILES: *c1cccc2c1n(C)c(=O)n2[C@H]1CCC(=O)NC1=O]

In some embodiments, LBM is

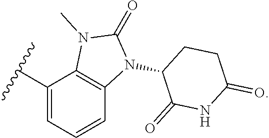 [SMILES: *c1cccc2c1n(C)c(=O)n2[C@@H]1CCC(=O)NC1=O]

In some embodiments, LBM is

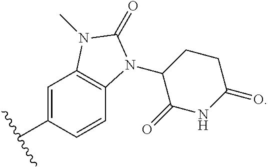 [SMILES: *c1ccc2c(c1)n(C)c(=O)n2C1CCC(=O)NC1=O]

In some embodiments, LBM is

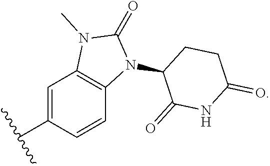 [SMILES: *c1ccc2c(c1)n(C)c(=O)n2[C@H]1CCC(=O)NC1=O]

In some embodiments, LBM is

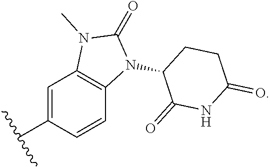 [SMILES: *c1ccc2c(c1)n(C)c(=O)n2[C@@H]1CCC(=O)NC1=O]

In some embodiments, LBM is

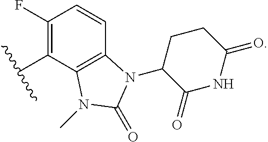 [SMILES: *c1c(F)ccc2c1n(C)c(=O)n2C1CCC(=O)NC1=O]

In some embodiments, LBM is

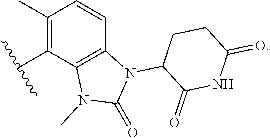 [SMILES: *c1c(C)ccc2c1n(C)c(=O)n2C1CCC(=O)NC1=O]

In some embodiments, LBM is

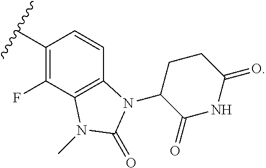 [SMILES: *c1ccc2c(c1F)n(C)c(=O)n2C1CCC(=O)NC1=O]

In some embodiments, LBM is

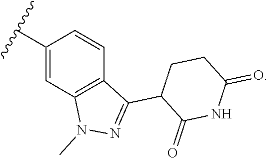 [SMILES: *c1ccc2c(C3CCC(=O)NC3=O)nn(C)c2c1]

In some embodiments, LBM is

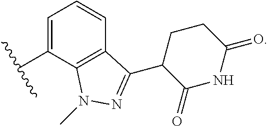 [SMILES: *c1cccc2c(C3CCC(=O)NC3=O)nn(C)c12]

In some embodiments, LBM is

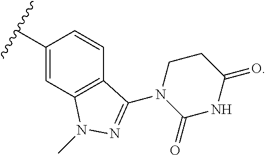 [SMILES: *c1ccc2c(N3CCC(=O)NC3=O)nn(C)c2c1]

In some embodiments, LBM is

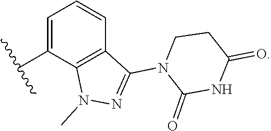 [SMILES: *c1cccc2c(N3CCC(=O)NC3=O)nn(C)c12]

In some embodiments, LBM is

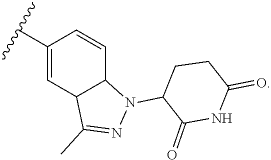 [SMILES: *C1=CC2C(C)=NN(C3CCC(=O)NC3=O)C2C=C1]

In some embodiments, LBM is

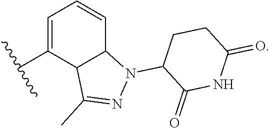 [SMILES: *C1=CC=CC2C1C(C)=NN2C1CCC(=O)NC1=O]

In some embodiments, LBM is

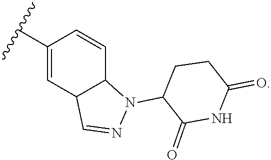 [SMILES: *C1=CC2C=NN(C3CCC(=O)NC3=O)C2C=C1]

In some embodiments, LBM is

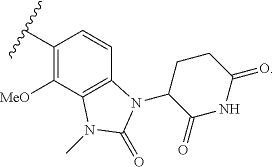 [SMILES: *c1ccc2c(c1OC)n(C)c(=O)n2C1CCC(=O)NC1=O]

In some embodiments, LBM is

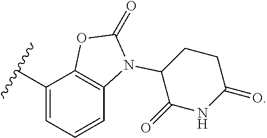 [SMILES: *c1cccc2c1oc(=O)n2C1CCC(=O)NC1=O]

In some embodiments, LBM is

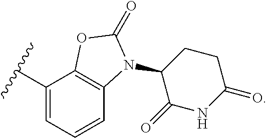 [SMILES: *c1cccc2c1oc(=O)n2[C@H]1CCC(=O)NC1=O]

In some embodiments, LBM is

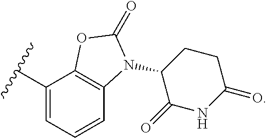 [SMILES: *c1cccc2c1oc(=O)n2[C@@H]1CCC(=O)NC1=O]

In some embodiments, LBM is

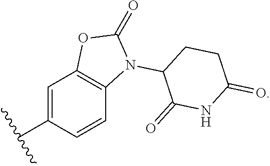 [SMILES: *c1ccc2c(c1)oc(=O)n2C1CCC(=O)NC1=O]

In some embodiments, LBM is

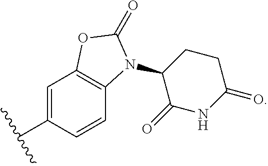 [SMILES: *c1ccc2c(c1)oc(=O)n2[C@H]1CCC(=O)NC1=O]

In some embodiments, LBM is

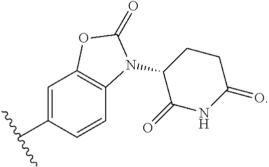 [SMILES: *c1ccc2c(c1)oc(=O)n2[C@@H]1CCC(=O)NC1=O]

In some embodiments, LBM is

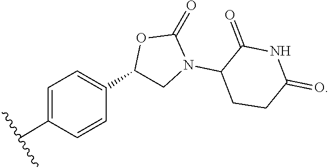 [SMILES: *c1ccc([C@H]2CN(C3CCC(=O)NC3=O)C(=O)O2)cc1]

In some embodiments, LBM is

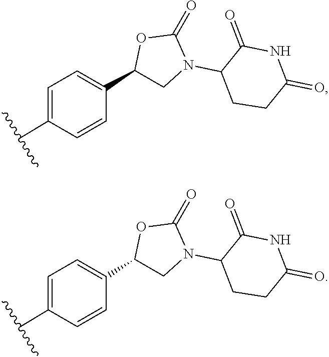 [SMILES: *c1ccc([C@@H]2CN(C3CCC(=O)NC3=O)C(=O)O2)cc1 ; *c1ccc([C@H]2CN(C3CCC(=O)NC3=O)C(=O)O2)cc1]

In some embodiments, LBM is

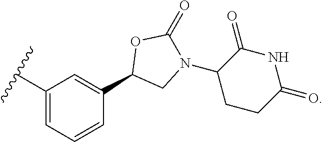 [SMILES: *c1cccc([C@@H]2CN(C3CCC(=O)NC3=O)C(=O)O2)c1]

In some embodiments, LBM is

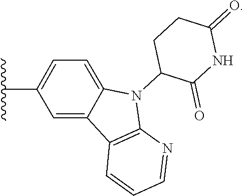 [SMILES: *c1ccc2c(c1)c1cccnc1n2C1CCC(=O)NC1=O]

In some embodiments, LBM is

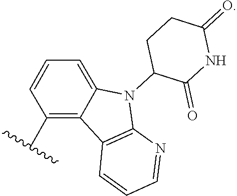 [SMILES: *c1cccc2c1c1cccnc1n2C1CCC(=O)NC1=O]

In some embodiments, LBM is

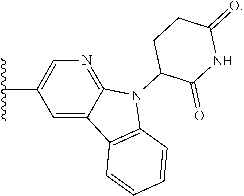 [SMILES: *c1cnc2c(c1)c1ccccc1n2C1CCC(=O)NC1=O]

In some embodiments, LBM is

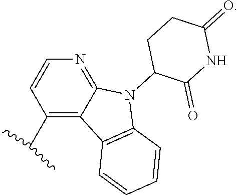 [SMILES: *c1ccnc2c1c1ccccc1n2C1CCC(=O)NC1=O]

In some embodiments, LBM is

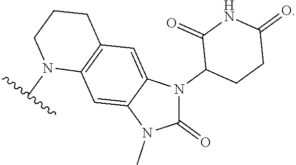 [SMILES: *N1CCCc2cc3c(cc21)n(C)c(=O)n3C1CCC(=O)NC1=O]

In some embodiments, LBM is

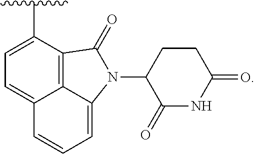 [SMILES: *c1ccc2cccc3c2c1C(=O)N3C1CCC(=O)NC1=O]

In some embodiments, LBM is

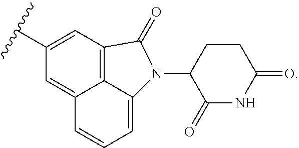 [SMILES: *c1cc2c3c(cccc3c1)N(C1CCC(=O)NC1=O)C2=O]

In some embodiments, LBM is

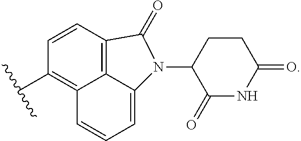 [SMILES: *c1ccc2c3c(cccc13)N(C1CCC(=O)NC1=O)C2=O]

In some embodiments, LBM is

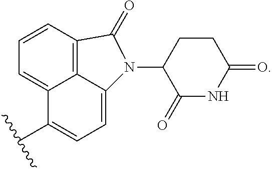 [SMILES: *c1ccc2c3c(cccc13)C(=O)N2C1CCC(=O)NC1=O]

In some embodiments, LBM is

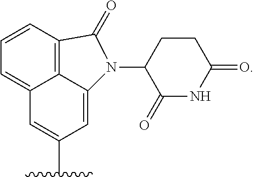 [SMILES: *c1cc2c3c(cccc3c1)C(=O)N2C1CCC(=O)NC1=O]

In some embodiments, LBM is

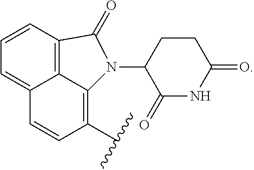 [SMILES: *c1ccc2cccc3c2c1N(C1CCC(=O)NC1=O)C3=O]

In some embodiments, LBM is

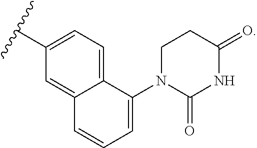 [SMILES: *c1ccc2c(N3CCC(=O)NC3=O)cccc2c1]

In some embodiments, LBM is

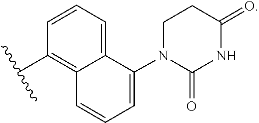 [SMILES: *c1cccc2c(N3CCC(=O)NC3=O)cccc12]

In some embodiments, LBM is

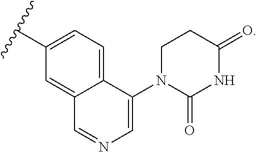 [SMILES: *c1ccc2c(N3CCC(=O)NC3=O)cncc2c1]

In some embodiments, LBM is

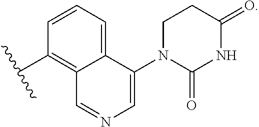 [SMILES: *c1cccc2c(N3CCC(=O)NC3=O)cncc12]

In some embodiments, LBM is

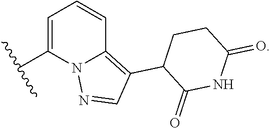 [SMILES: *c1cccc2c(C3CCC(=O)NC3=O)cnn12]

In some embodiments, LBM is

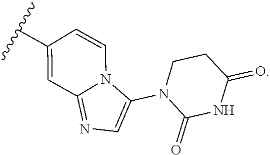 [SMILES: *c1ccn2c(N3CCC(=O)NC3=O)cnc2c1]

In some embodiments, LBM is

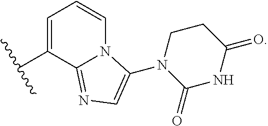 [SMILES: *c1cccn2c(N3CCC(=O)NC3=O)cnc12]

In some embodiments, LBM is

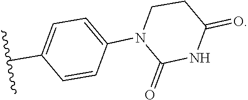 [SMILES: *c1ccc(N2CCC(=O)NC2=O)cc1]

In some embodiments, LBM is

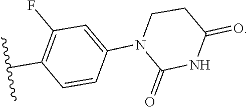 [SMILES: *c1ccc(N2CCC(=O)NC2=O)cc1F]

In some embodiments, LBM is

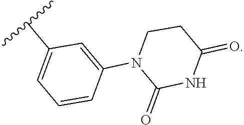 [SMILES: *c1cccc(N2CCC(=O)NC2=O)c1]

In some embodiments, LBM is

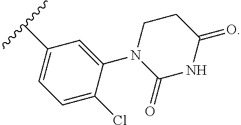 [SMILES: *c1ccc(Cl)c(N2CCC(=O)NC2=O)c1]

In some embodiments, LBM is

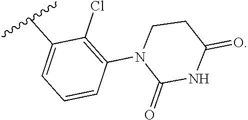 [SMILES: *c1cccc(N2CCC(=O)NC2=O)c1Cl]

In some embodiments, LBM is

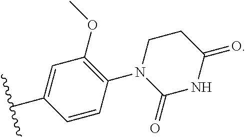 [SMILES: *c1ccc(N2CCC(=O)NC2=O)c(OC)c1]

In some embodiments, LBM is

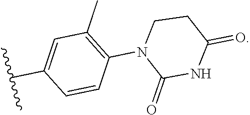 [SMILES: *c1ccc(N2CCC(=O)NC2=O)c(C)c1]

In some embodiments, LBM is

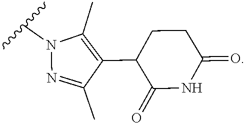 [SMILES: *n1nc(C)c(C2CCC(=O)NC2=O)c1C]

In some embodiments, LBM is

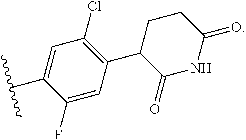 [SMILES: *c1cc(Cl)c(C2CCC(=O)NC2=O)cc1F]

In some embodiments, LBM is

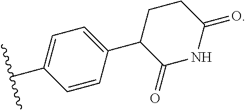 [SMILES: *c1ccc(C2CCC(=O)NC2=O)cc1]

In some embodiments, LBM is

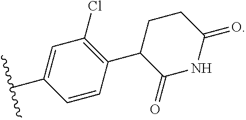 [SMILES: *c1ccc(C2CCC(=O)NC2=O)c(Cl)c1]

In some embodiments, LBM is

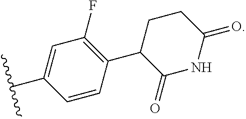 [SMILES: *c1ccc(C2CCC(=O)NC2=O)c(F)c1]

In some embodiments, LBM is

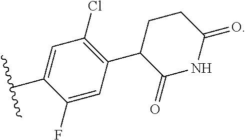 [SMILES: *c1cc(Cl)c(C2CCC(=O)NC2=O)cc1F]

In some embodiments, LBM is

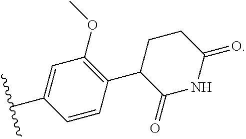 [SMILES: *c1ccc(C2CCC(=O)NC2=O)c(OC)c1]

In some embodiments, LBM is

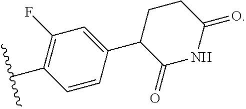 [SMILES: *c1ccc(C2CCC(=O)NC2=O)cc1F]

In some embodiments, LBM is

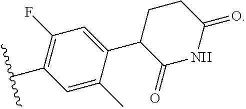 [SMILES: *c1cc(C)c(C2CCC(=O)NC2=O)cc1F]

In some embodiments, LBM is

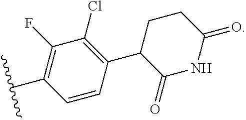 [SMILES: *c1ccc(C2CCC(=O)NC2=O)c(Cl)c1F]

In some embodiments, LBM is

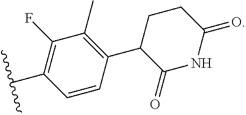 [SMILES: *c1ccc(C2CCC(=O)NC2=O)c(C)c1F]

In some embodiments, LBM is

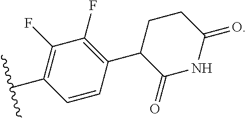 [SMILES: *c1ccc(C2CCC(=O)NC2=O)c(F)c1F]

In some embodiments, LBM is

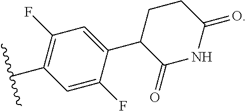 [SMILES: *c1cc(F)c(C2CCC(=O)NC2=O)cc1F]

In some embodiments, LBM is

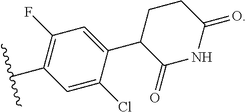 [SMILES: *c1cc(Cl)c(C2CCC(=O)NC2=O)cc1F]

In some embodiments, LBM is

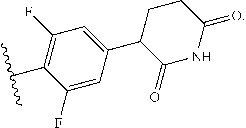 [SMILES: *c1c(F)cc(C2CCC(=O)NC2=O)cc1F]

In some embodiments, LBM is

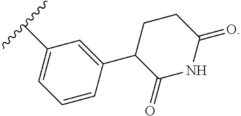 [SMILES: *c1cccc(C2CCC(=O)NC2=O)c1]

In some embodiments, LBM is

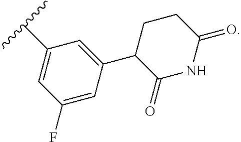 [SMILES: *c1cc(F)cc(C2CCC(=O)NC2=O)c1]

In some embodiments, LBM is

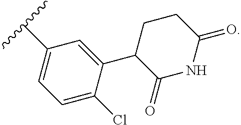 [SMILES: *c1ccc(Cl)c(C2CCC(=O)NC2=O)c1]

In some embodiments, LBM is

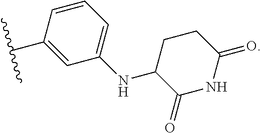 [SMILES: *c1cccc(NC2CCC(=O)NC2=O)c1]

In some embodiments, LBM is

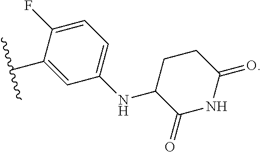 [SMILES: *c1cc(NC2CCC(=O)NC2=O)ccc1F]

In some embodiments, LBM is

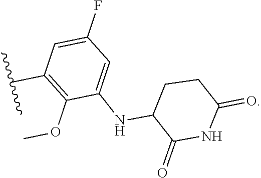 [SMILES: *c1cc(F)cc(NC2CCC(=O)NC2=O)c1OC]

In some embodiments, LBM is

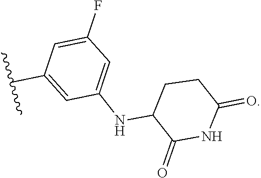 [SMILES: *c1cc(F)cc(NC2CCC(=O)NC2=O)c1]

In some embodiments, LBM is

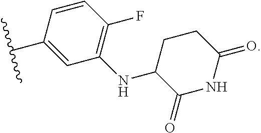 [SMILES: *c1ccc(F)c(NC2CCC(=O)NC2=O)c1]

In some embodiments, LBM is

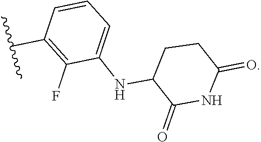 [SMILES: *c1cccc(NC2CCC(=O)NC2=O)c1F]

In some embodiments, LBM is

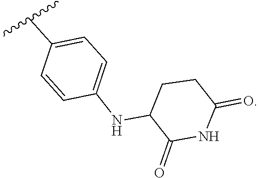 [SMILES: *c1ccc(NC2CCC(=O)NC2=O)cc1]

In some embodiments, LBM is

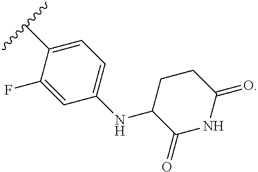 [SMILES: *c1ccc(NC2CCC(=O)NC2=O)cc1F]

In some embodiments, LBM is

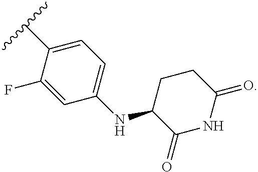 [SMILES: *c1ccc(N[C@H]2CCC(=O)NC2=O)cc1F]

In some embodiments, LBM is

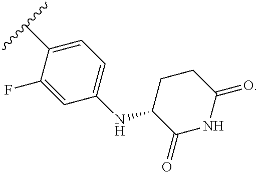 [SMILES: *c1ccc(N[C@@H]2CCC(=O)NC2=O)cc1F]

In some embodiments, LBM is

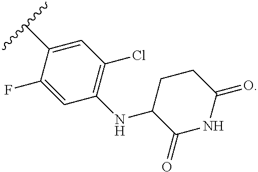 [SMILES: *c1cc(Cl)c(NC2CCC(=O)NC2=O)cc1F]

In some embodiments, LBM is

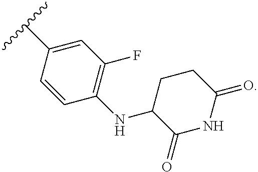 [SMILES: *c1ccc(NC2CCC(=O)NC2=O)c(F)c1]

In some embodiments, LBM is

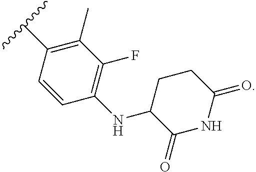 [SMILES: *c1ccc(NC2CCC(=O)NC2=O)c(F)c1C]

In some embodiments, LBM is

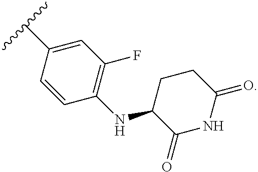 [SMILES: *c1ccc(N[C@H]2CCC(=O)NC2=O)c(F)c1]

In some embodiments, LBM is

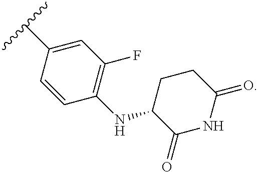 [SMILES: *c1ccc(N[C@@H]2CCC(=O)NC2=O)c(F)c1]

In some embodiments, LBM is

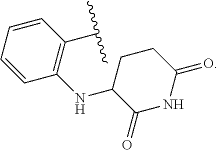 [SMILES: *c1ccccc1NC1CCC(=O)NC1=O]

In some embodiments, LBM is

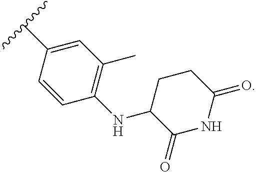 [SMILES: *c1ccc(NC2CCC(=O)NC2=O)c(C)c1]

In some embodiments, LBM is

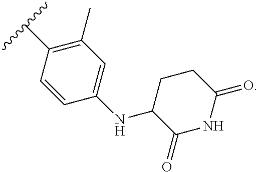 [SMILES: *c1ccc(NC2CCC(=O)NC2=O)cc1C]

In some embodiments, LBM is

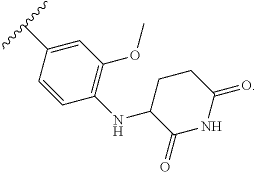 [SMILES: *c1ccc(NC2CCC(=O)NC2=O)c(OC)c1]

In some embodiments, LBM is

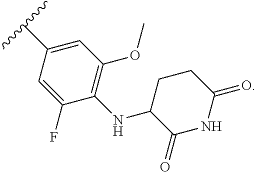 [SMILES: *c1cc(F)c(NC2CCC(=O)NC2=O)c(OC)c1]

In some embodiments, LBM is

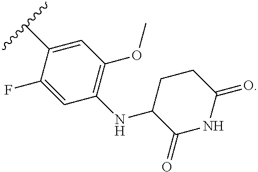 [SMILES: *c1cc(OC)c(NC2CCC(=O)NC2=O)cc1F]

In some embodiments, LBM is

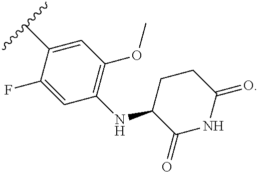 [SMILES: *c1cc(OC)c(N[C@H]2CCC(=O)NC2=O)cc1F]

In some embodiments, LBM is

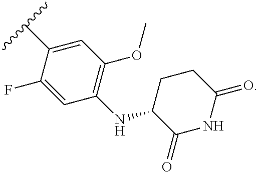 [SMILES: *c1cc(OC)c(N[C@@H]2CCC(=O)NC2=O)cc1F]

In some embodiments, LBM is

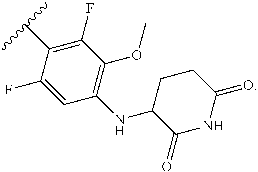 [SMILES: *c1c(F)cc(NC2CCC(=O)NC2=O)c(OC)c1F]

In some embodiments, LBM is

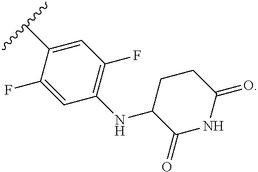 [SMILES: *c1cc(F)c(NC2CCC(=O)NC2=O)cc1F]

In some embodiments, LBM is

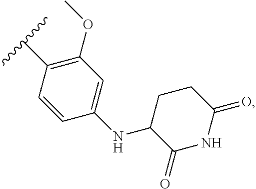 [SMILES: *c1ccc(NC2CCC(=O)NC2=O)cc1OC]

In some embodiments, LBM is

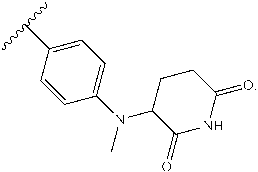 [SMILES: *c1ccc(N(C)C2CCC(=O)NC2=O)cc1]

In some embodiments, LBM is

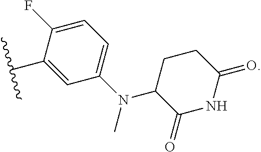 [SMILES: *c1cc(N(C)C2CCC(=O)NC2=O)ccc1F]

In some embodiments, LBM is

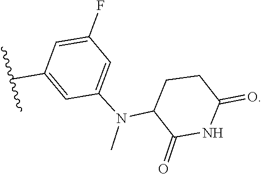 [SMILES: *c1cc(F)cc(N(C)C2CCC(=O)NC2=O)c1]

In some embodiments, LBM is

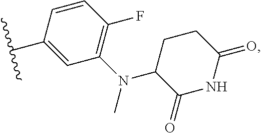 [SMILES: *c1ccc(F)c(N(C)C2CCC(=O)NC2=O)c1]

In some embodiments, LBM is

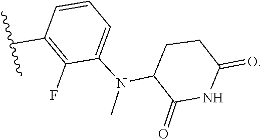 [SMILES: *c1cccc(N(C)C2CCC(=O)NC2=O)c1F]

In some embodiments, LBM is

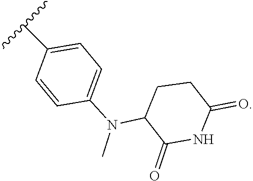 [SMILES: *c1ccc(N(C)C2CCC(=O)NC2=O)cc1]

In some embodiments, LBM is

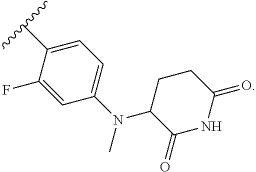 [SMILES: *c1ccc(N(C)C2CCC(=O)NC2=O)cc1F]

In some embodiments, LBM is

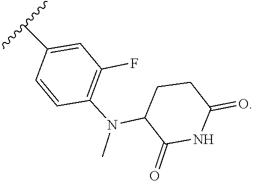 [SMILES: *c1ccc(N(C)C2CCC(=O)NC2=O)c(F)c1]

In some embodiments, LBM is

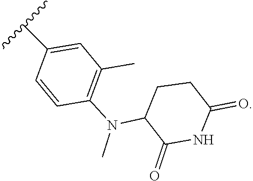 [SMILES: *c1ccc(N(C)C2CCC(=O)NC2=O)c(C)c1]

In some embodiments, LBM is

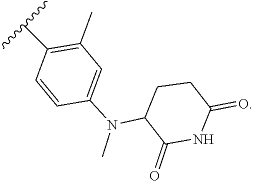 [SMILES: *c1ccc(N(C)C2CCC(=O)NC2=O)cc1C]

In some embodiments, LBM is

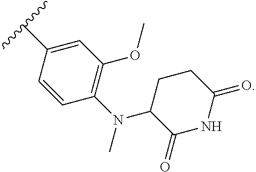 [SMILES: *c1ccc(N(C)C2CCC(=O)NC2=O)c(OC)c1]

In some embodiments, LBM is

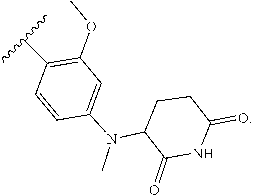 [SMILES: *c1ccc(N(C)C2CCC(=O)NC2=O)cc1OC]

In some embodiments, LBM is

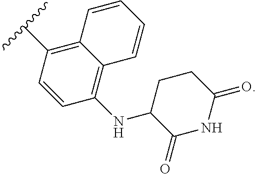 [SMILES: *c1ccc(NC2CCC(=O)NC2=O)c2ccccc12]

In some embodiments, LBM is

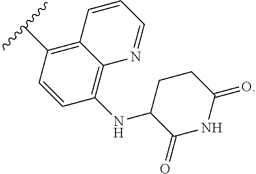 [SMILES: *c1ccc(NC2CCC(=O)NC2=O)c2ncccc12]

In some embodiments, LBM is

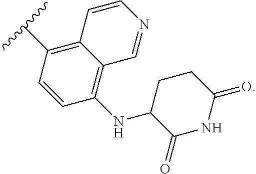 [SMILES: *c1ccc(NC2CCC(=O)NC2=O)c2cnccc12]

In some embodiments, LBM is

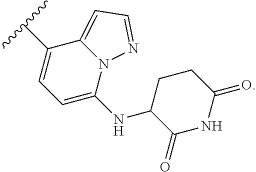 [SMILES: *c1ccc(NC2CCC(=O)NC2=O)n2nccc12]

In some embodiments, LBM is

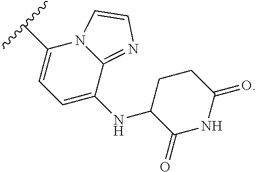 [SMILES: *c1ccc(NC2CCC(=O)NC2=O)c2nccn12]

In some embodiments, LBM is

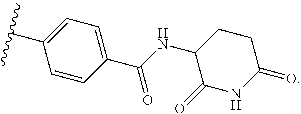 [SMILES: *c1ccc(C(=O)NC2CCC(=O)NC2=O)cc1]

In some embodiments, LBM is

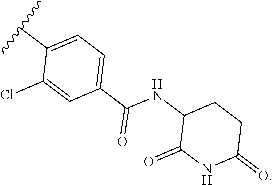 [SMILES: *c1ccc(C(=O)NC2CCC(=O)NC2=O)cc1Cl]

In some embodiments, LBM is

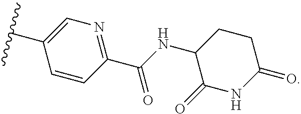 [SMILES: *c1ccc(C(=O)NC2CCC(=O)NC2=O)nc1]

In some embodiments, LBM is

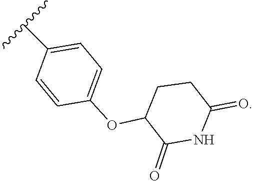 [SMILES: *c1ccc(OC2CCC(=O)NC2=O)cc1]

In some embodiments, LBM is

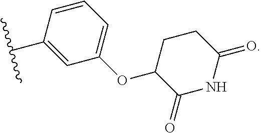 [SMILES: *c1cccc(OC2CCC(=O)NC2=O)c1]

In some embodiments, LBM is

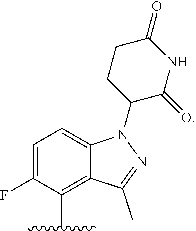 [SMILES: *c1c(F)ccc2c1c(C)nn2C1CCC(=O)NC1=O]

In some embodiments, LBM is

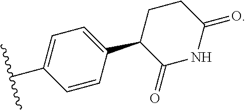 [SMILES: *c1ccc([C@H]2CCC(=O)NC2=O)cc1]

In some embodiments, LBM is

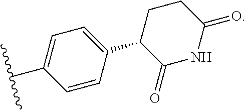 [SMILES: *c1ccc([C@@H]2CCC(=O)NC2=O)cc1]

In some embodiments, LBM is

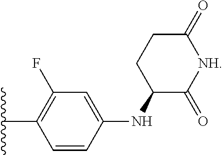 [SMILES: *c1ccc(N[C@H]2CCC(=O)NC2=O)cc1F]

In some embodiments, LBM is

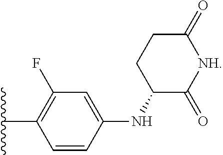 [SMILES: *c1ccc(N[C@@H]2CCC(=O)NC2=O)cc1F]

In certain embodiments, the present invention provides a compound of formula I wherein SBM is a compound of formula I-a and LBM is a formula of I-aa thereby forming a compound of formula I-aa-1:

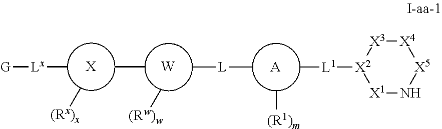

or a pharmaceutically acceptable salt thereof, wherein L, L^{x}, L¹, Ring A, Ring W, Ring X, G, R^{w}, R^{x}, R¹, X¹, X², X³, X⁴, X⁵, m, w, and x are as defined above and described herein both individually and in combination.

In certain embodiments, the present invention provides a compound of formula I-aa-1 as a compound of any one of the following formulae:

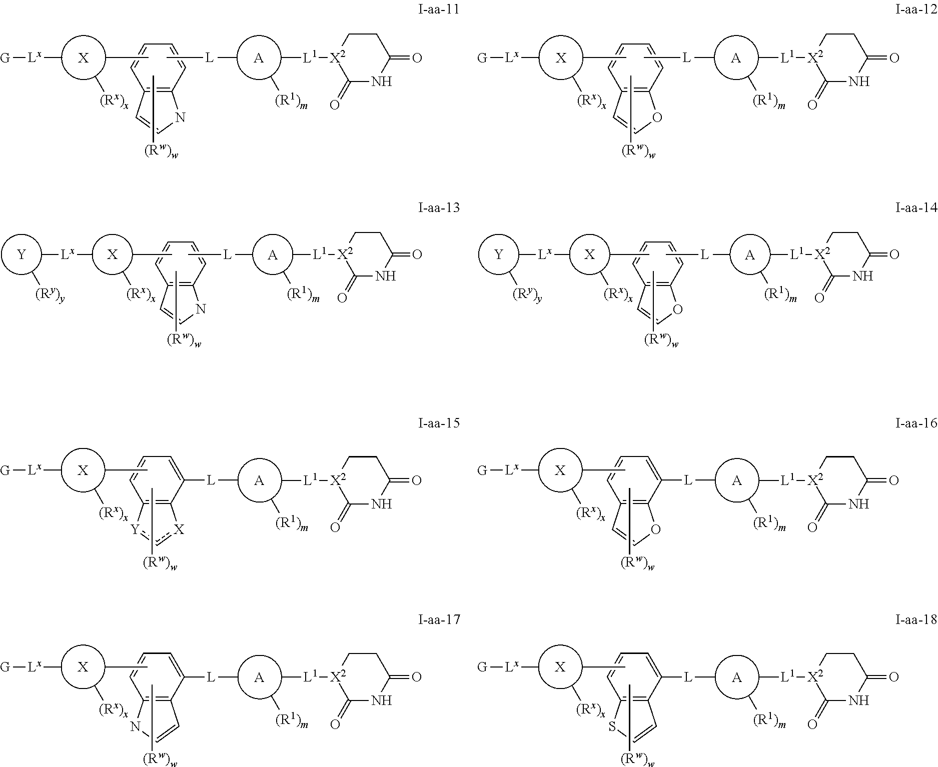

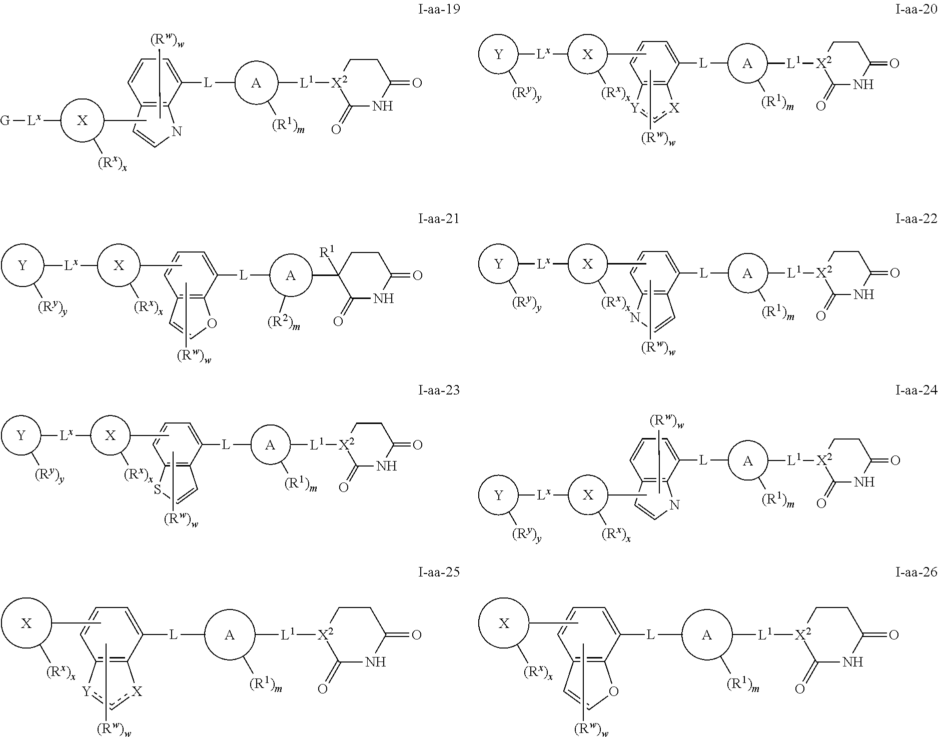

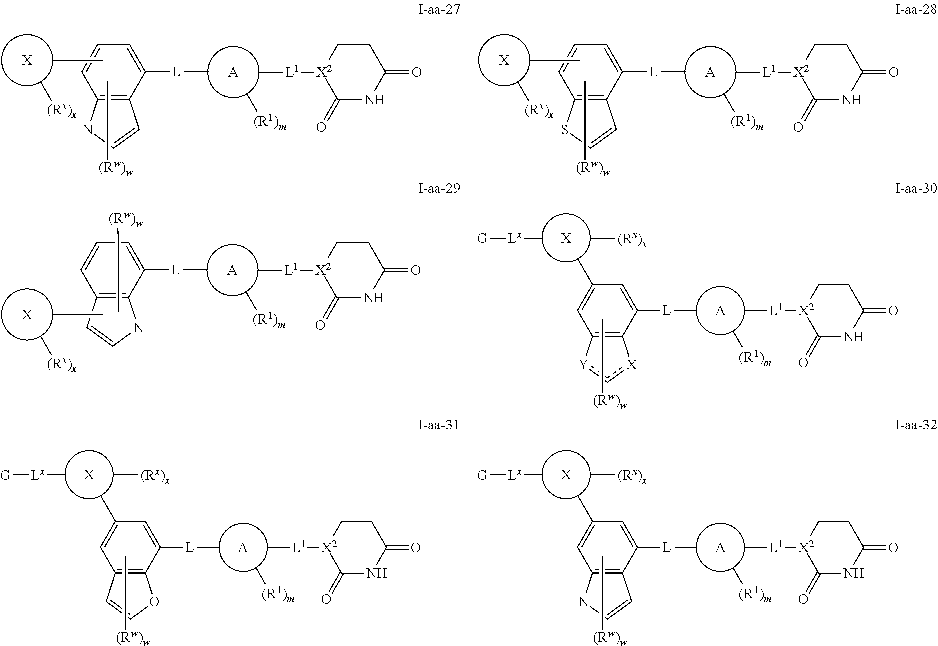

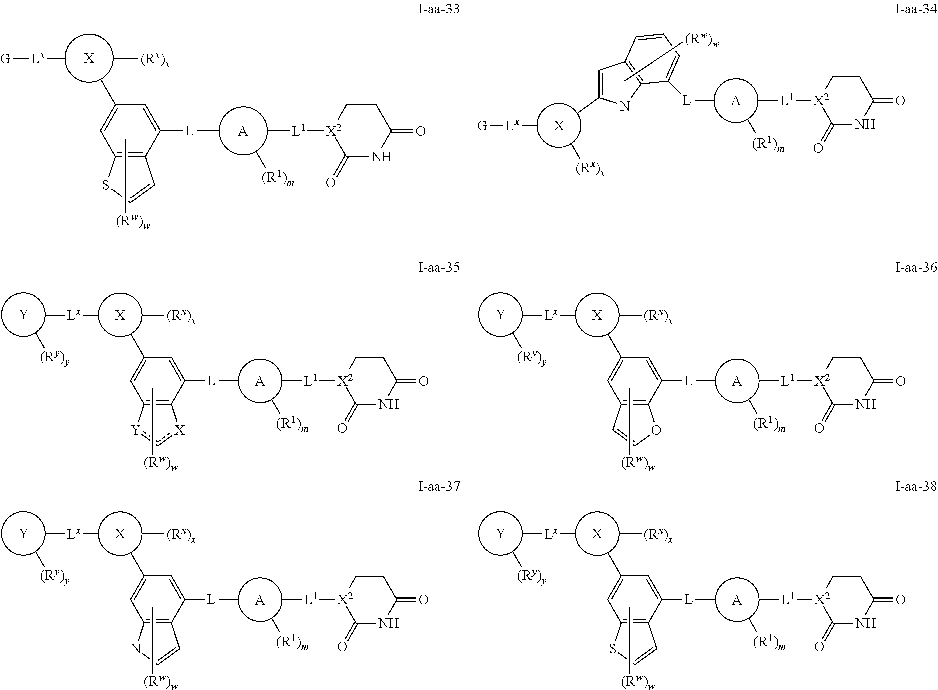

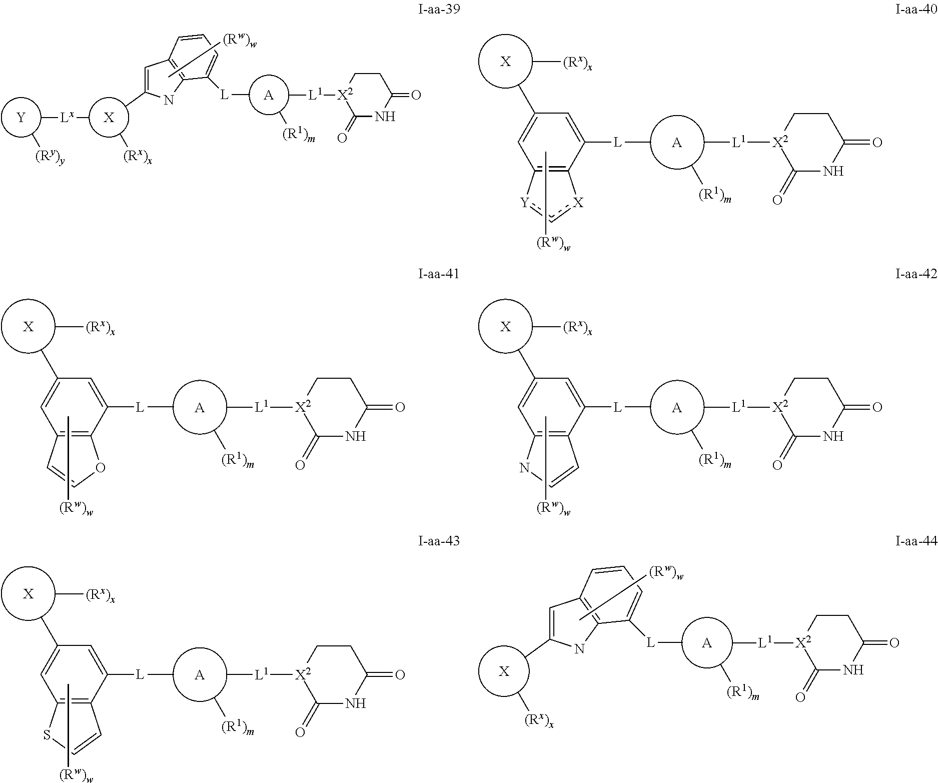

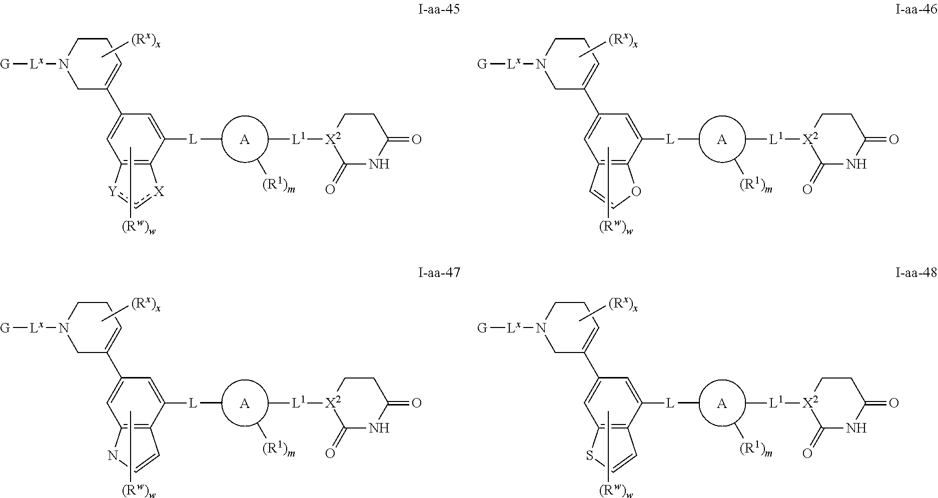

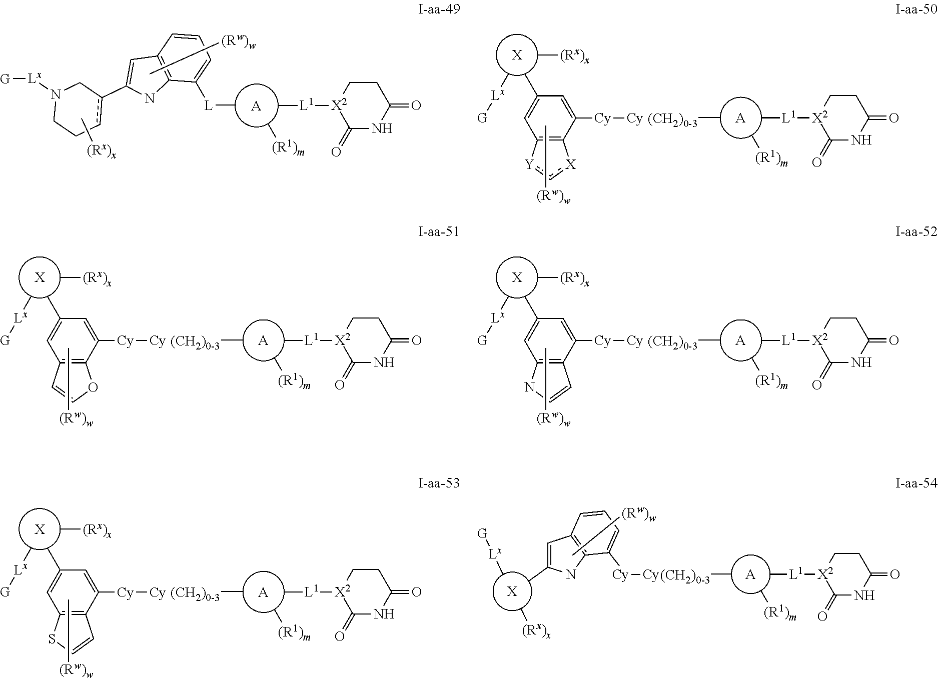

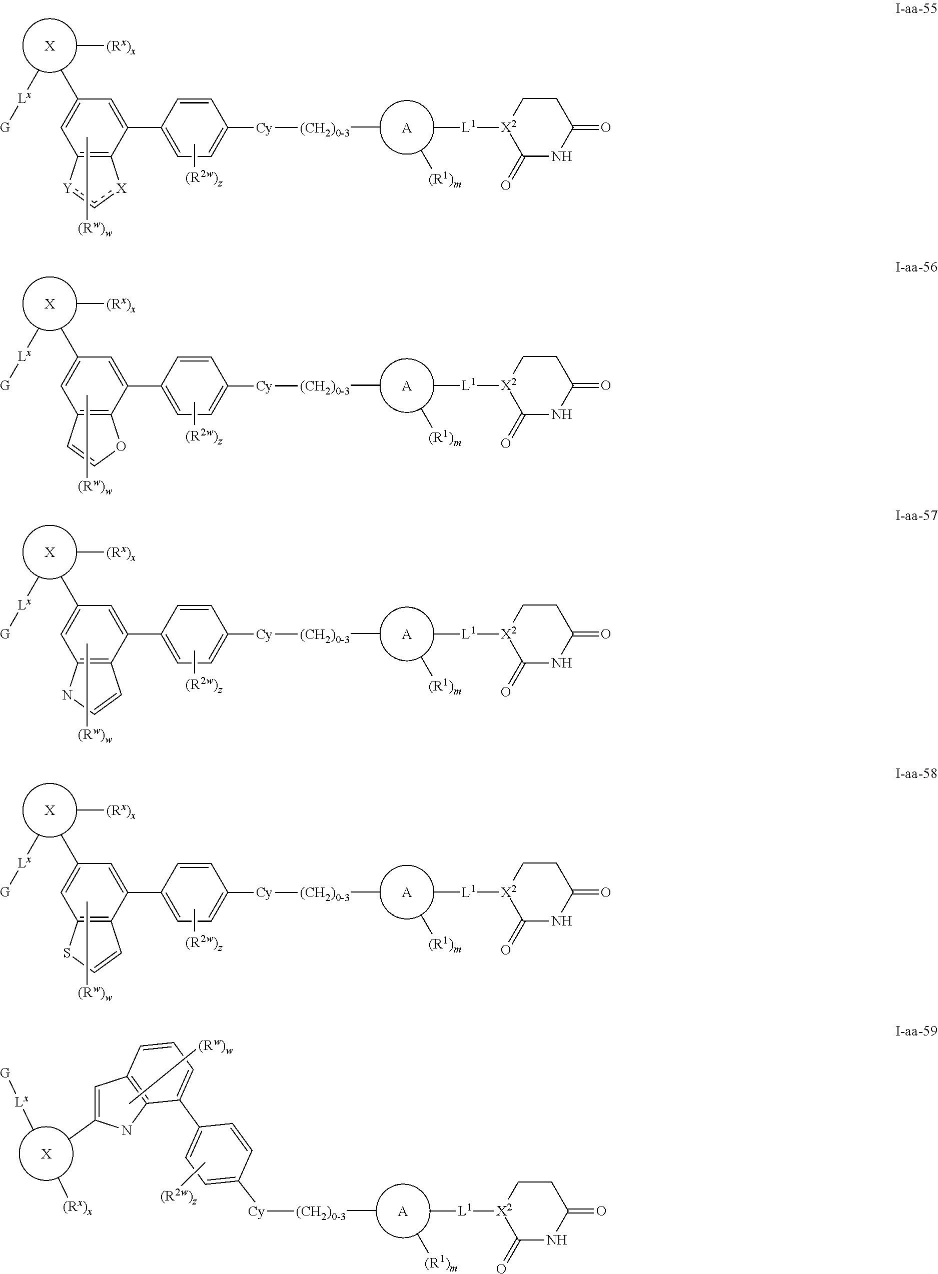

or a pharmaceutically acceptable salt thereof, wherein each variable is as defined above and described herein both individually and in combination.

In some embodiments, the present invention provides a compound of formula I, wherein LBM is an E3 ubiquitin ligase (cereblon) binding moiety thereby forming a compound of formula I-oo-1, I-oo-2, I-oo-3, I-oo-4, I-oo-5, I-oo-6. I-oo-7, I-oo-8, I-oo-9, or I-oo-10 respectively:

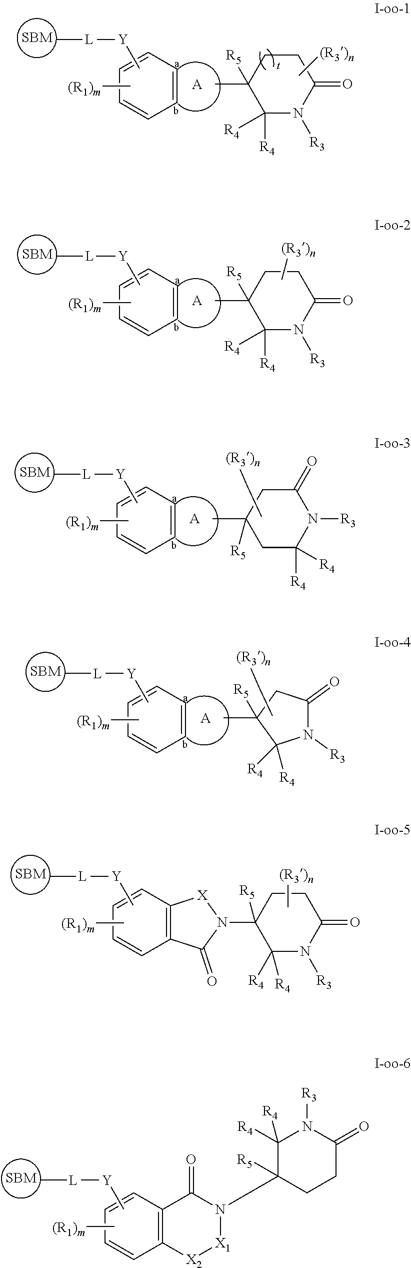

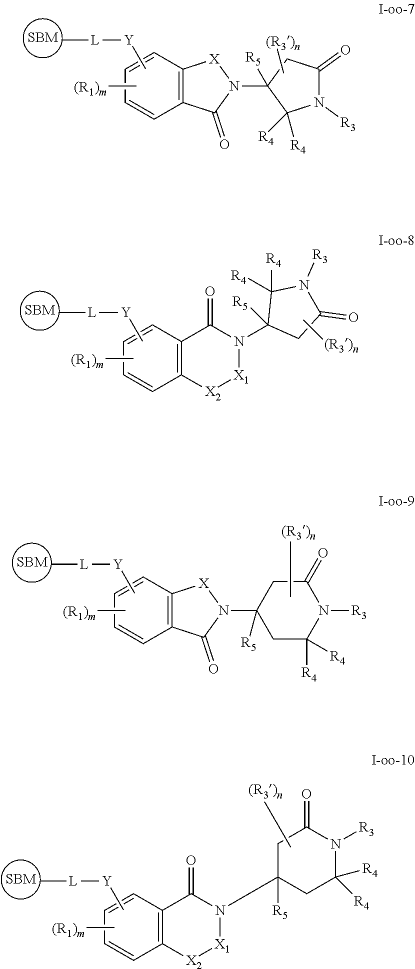

or a compound of formula I-oo′-1, I-oo′-2. I-oo′-3, I-oo′-4, I-oo′-5, I-oo′-6, I-oo′-7. I-oo′-8, I-oo′-9, or I-oo′-10 respectively:

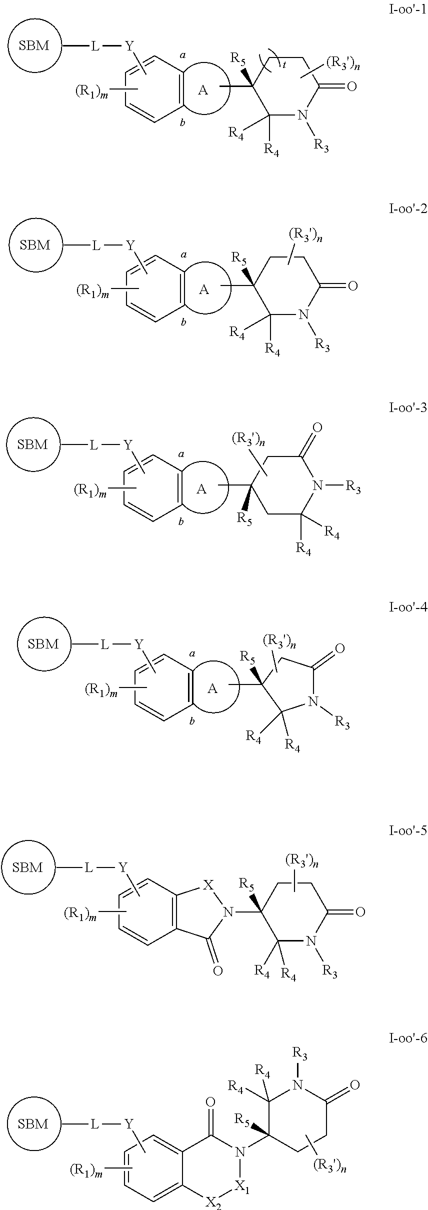

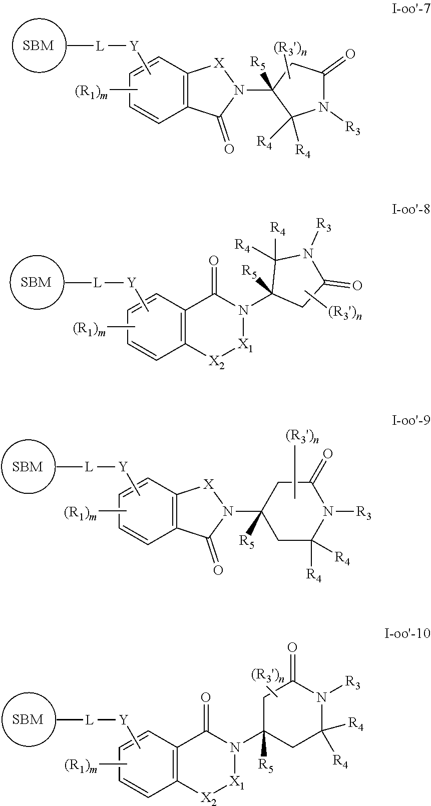

or a compound of formula I-oo″-1, I-oo″-2, I-oo″-3, I-oo″-4, I-oo″-5, I-oo″-6, I-oo″-7, I-oo″-8, I-oo″-9, or I-oo″-10 respectively:

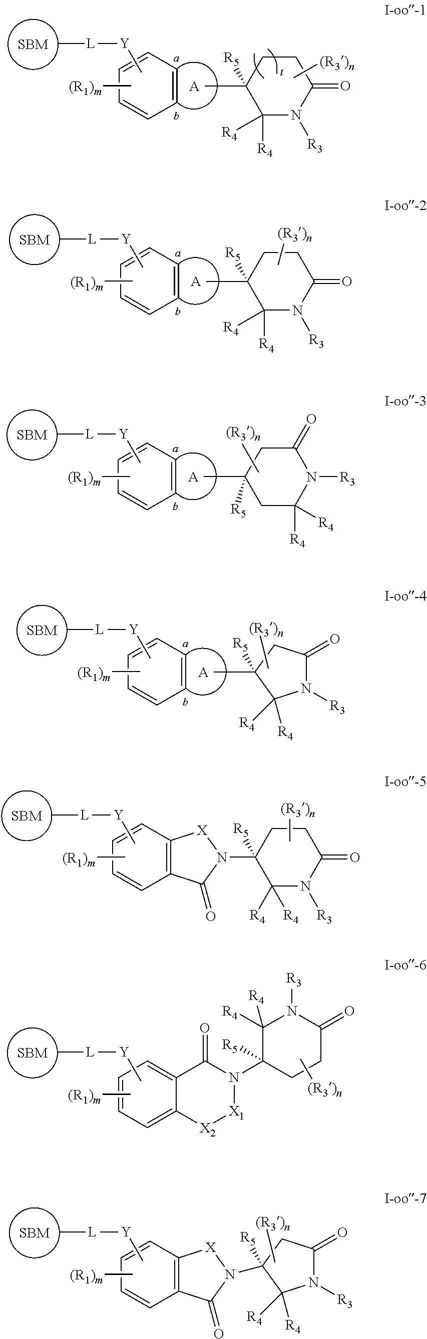

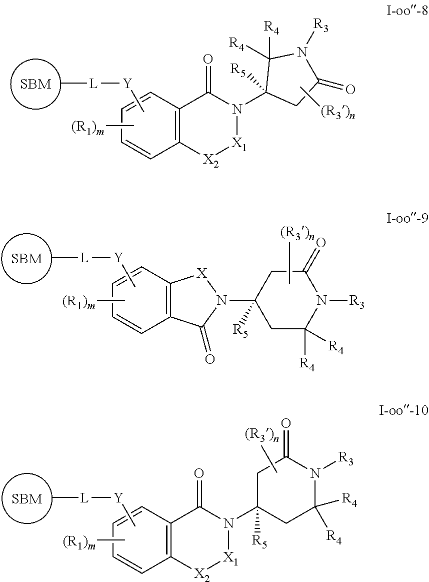

or a pharmaceutically acceptable salt thereof, wherein:

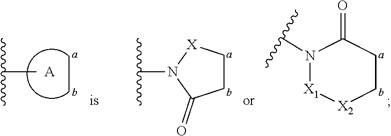

Y is a bond, Y₁, O, NH, NR₂, C(O)O, OC(O), C(O)NR₂′, NR₂′C(O), Y₁—O, Y₁—NH, Y₁—NR₂, Y₁—C(O), Y₁—C(O)O, Y₁—OC(O), Y₁—C(O)NR₂′, or Y₁—NR₂′C(O), wherein Y₁ is C₁-C₆ alkylene, C₂-C₆ alkenylene, or C₂-C₆ alkynylene;
X is C(O) or C(R³)₂;
X₁—X₂ is C(R₃)=N or C(R³)₂—C(R³)₂;
each R₁ is independently halogen, nitro, NH₂, OH, C(O)OH, C₁-C₆ alkyl, or C₁-C₆ alkoxy;
R₂ is C₁-C₆ alkyl, C₂-C₆ alkenyl, C₃-C₈ cycloalkyl, 3- to 8-membered heterocycloalkyl, C(O)—C₁-C₆ alkyl, C(O)—C₂-C₆ alkenyl, C(O)—C₃-C₈ cycloalkyl, or C(O)-3- to 8-membered heterocycloalkyl, and R₂ is optionally substituted with one or more of halogen, N(Rₐ)₂, NHC(O)Rₐ, NHC(O)ORₐ, OR_{b}, C₃-C₈ cycloalkyl, 3- to 8-membered heterocycloalkyl, C₆-C₁₀ aryl, or 5- to 10-membered heteroaryl, wherein each of the C₃-C₈ cycloalkyl, 3- to 8-membered heterocycloalkyl, C₆-C₁₀ aryl or 5- to 10-membered heteroaryl is optionally further substituted with one or more of halogen, NH₂. CN, nitro, OH, C(O)OH, C₁-C₆alkyl, C₁-C₆haloalkyl, C₁-C₆ alkoxy, or C₁-C₆ haloalkoxy;
R₂′ is H, C₁-C₆ alkyl, C₂-C₆ alkenyl, C₃-C₈ cycloalkyl, or 3- to 8-membered heterocycloalkyl, and R₂′, when not being H, is optionally substituted with one or more of halogen, N(Rₐ)₂, NHC(O)Rₐ, NHC(O)ORₐ, OR_{b}, C₃-C₈ cycloalkyl, 3- to 8-membered heterocycloalkyl, C₆-C₁₀ aryl, or 5- to 10-membered heteroaryl, wherein each of the C₃-C₈ cycloalkyl, 3- to 8-membered heterocycloalkyl, C₆-C₁₀ aryl or 5- to 10-membered heteroaryl is optionally further substituted with one or more of halogen, NH₂, CN, nitro, OH, C(O)OH, C₁-C₆alkyl, C₁-C₆haloalkyl, C₁-C₆alkoxy, or C₁-C₆ haloalkoxy:
each R₃ is independently H or C₁-C₃ alkyl optionally substituted with C₆-C₁₀ aryl or 5- to 10-membered heteroaryl;
each R₃′ is independently C₁-C₃ alkyl;
each R₄ is independently H or C₁-C₃ alkyl; or two R₄, together with the carbon atom to which they are attached, form C(O), a C₃-C₆ carbocycle, or a 4-, 5-, or 6-membered heterocycle comprising 1 or 2 heteroatoms selected from N and O;
R₅ is H, C₁-C₃ alkyl, F, or Cl;
each Rₐ independently is H or C₁-C₆ alkyl:
R_{b} is H or tosyl;
t is 0 or 1;
m is 0, 1, 2 or 3; and
n is 0, 1 or 2.

In some embodiments, LBM is

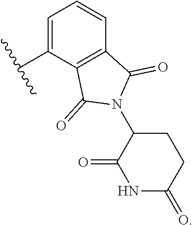 [SMILES: *c1cccc2c1C(=O)N(C1CCC(=O)NC1=O)C2=O]

In some embodiments, LBM is

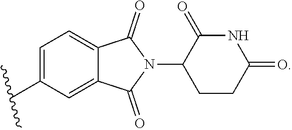 [SMILES: *c1ccc2c(c1)C(=O)N(C1CCC(=O)NC1=O)C2=O]

In some embodiments, LBM is

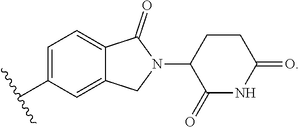 [SMILES: *c1ccc2c(c1)CN(C1CCC(=O)NC1=O)C2=O]

In some embodiments, LBM is

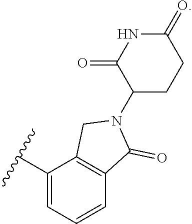 [SMILES: *c1cccc2c1CN(C1CCC(=O)NC1=O)C2=O]

In some embodiments, LBM is

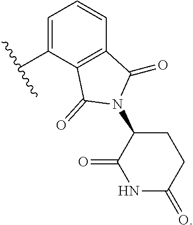 [SMILES: *c1cccc2c1C(=O)N([C@H]1CCC(=O)NC1=O)C2=O]

In some embodiments, LBM is

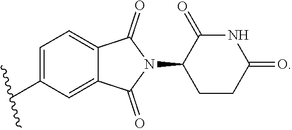 [SMILES: *c1ccc2c(c1)C(=O)N([C@@H]1CCC(=O)NC1=O)C2=O]

In some embodiments, LBM is

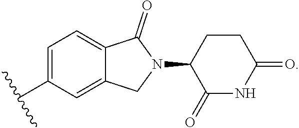 [SMILES: *c1ccc2c(c1)CN([C@H]1CCC(=O)NC1=O)C2=O]

In some embodiments, LBM is

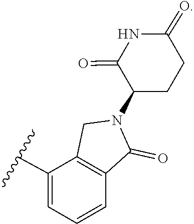 [SMILES: *c1cccc2c1CN([C@@H]1CCC(=O)NC1=O)C2=O]

In some embodiments, LBM is

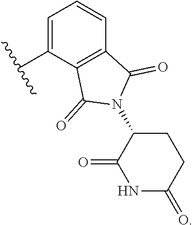 [SMILES: *c1cccc2c1C(=O)N([C@@H]1CCC(=O)NC1=O)C2=O]

In some embodiments, LBM is

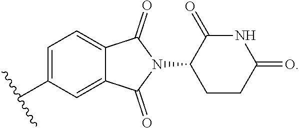 [SMILES: *c1ccc2c(c1)C(=O)N([C@H]1CCC(=O)NC1=O)C2=O]

In some embodiments, LBM is

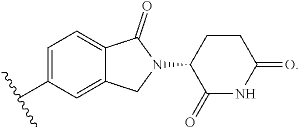 [SMILES: *c1ccc2c(c1)CN([C@@H]1CCC(=O)NC1=O)C2=O]

In some embodiments, LBM is

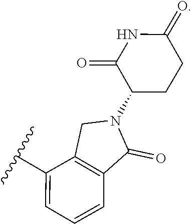 [SMILES: *c1cccc2c1CN([C@H]1CCC(=O)NC1=O)C2=O]

In some embodiments, LBM is

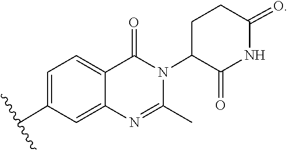 [SMILES: *c1ccc2c(=O)n(C3CCC(=O)NC3=O)c(C)nc2c1]

In some embodiments, LBM is

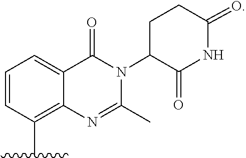 [SMILES: *c1cccc2c(=O)n(C3CCC(=O)NC3=O)c(C)nc12]

In some embodiments, LBM is

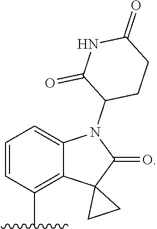 [SMILES: *c1cccc2c1C1(CC1)C(=O)N2C1CCC(=O)NC1=O]

In some embodiments, LBM is

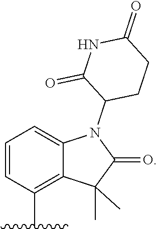 [SMILES: *c1cccc2c1C(C)(C)C(=O)N2C1CCC(=O)NC1=O]

In some embodiments, LBM is selected from those in Table 1 below.

In certain embodiments, the present invention provides a compound of formula I, wherein LBM is an E3 ubiquitin ligase (cereblon) binding moiety thereby forming a compound of formula I-uu:

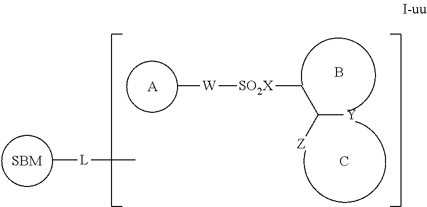

or a pharmaceutically acceptable salt thereof, wherein L and SBM are as defined above and described in embodiments herein, and wherein:
A represents a monocyclic or bicyclic aromatic ring which may be substituted;
B represents a six-membered unsaturated hydrocarbon ring or a six-membered unsaturated heterocycle containing one nitrogen atom as the heteroatom, each of which may be substituted;
C represents a five-membered heterocycle containing one or two nitrogen atoms which may be substituted:
W represents a single bond or a group represented by formula —CH═CH—;
X represents a group represented by formula —N(R¹)— or oxygen;
Y represents carbon or nitrogen;
Z represents a group represented by formula —N(R²)— or nitrogen; and
R¹ and R² may be the same or different from each other and each represent hydrogen or lower alkyl; as described and defined in U.S. Pat. No. 5,721,246, the entirety of each of which is herein incorporated by reference.

In some embodiments, LBM is a IAP E3 Ubiquitin ligase binding moiety recited in Varfolomeev, E. et al., IAP Antagonists Induce Autoubiquitination of c-IAPs, NF-κB activation, and TNFα-Dependent Apoptosis, Cell, 2007, 131(4): 669-81, such as, for example:

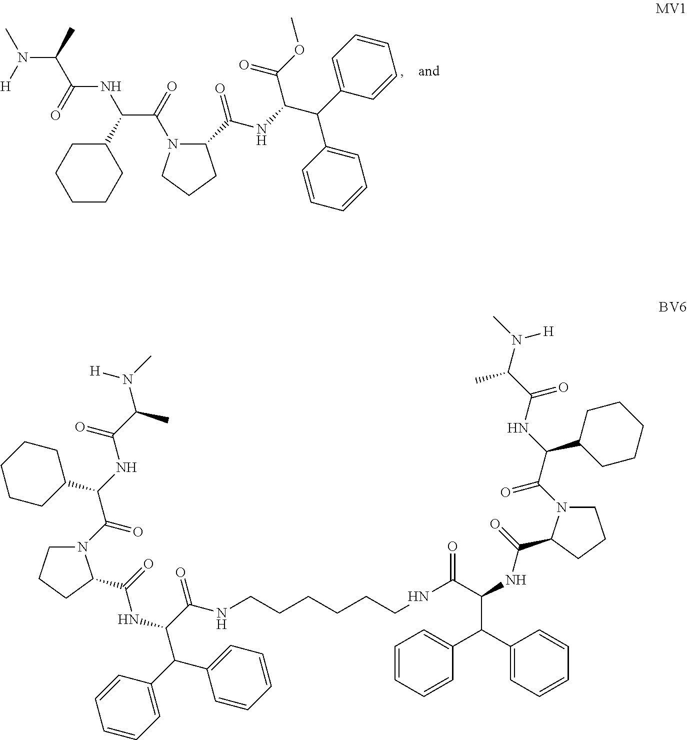 [SMILES: CN[C@@H](C)C(=O)N[C@H](C(=O)N1CCC[C@H]1C(=O)N[C@H](C(=O)OC)C(c1ccccc1)c1ccccc1)C1CCCCC1 ; CN[C@@H](C)C(=O)N[C@H](C(=O)N1CCC[C@H]1C(=O)N[C@H](C(=O)NCCCCCCNC(=O)[C@@H](NC(=O)[C@@H]1CCCN1C(=O)[C@@H](NC(=O)[C@H](C)NC)C1CCCCC1)C(c1ccccc1)c1ccccc1)C(c1ccccc1)c1ccccc1)C1CCCCC1]

wherein

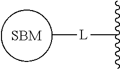

is attached to a modifiable carbon, oxygen, nitrogen or sulfur atom.

In certain embodiments, the present invention provides a compound of Formula I, wherein LBM is a MDM2 (i.e. human double minute 2 or HDM2) E3 ligase binding moiety thereby forming a compound of formula I-aaa-1, I-aaa-2, I-aaa-3, I-aaa-4, I-aaa-5, I-aaa-6, I-aaa-7, I-aaa-8, I-aaa-9, I-aaa-10, I-aaa-11, I-aaa-12, I-aaa-13, I-aaa-14. I-aaa-15. I-aaa-16, I-aaa-17, or I-aaa-18 respectively:

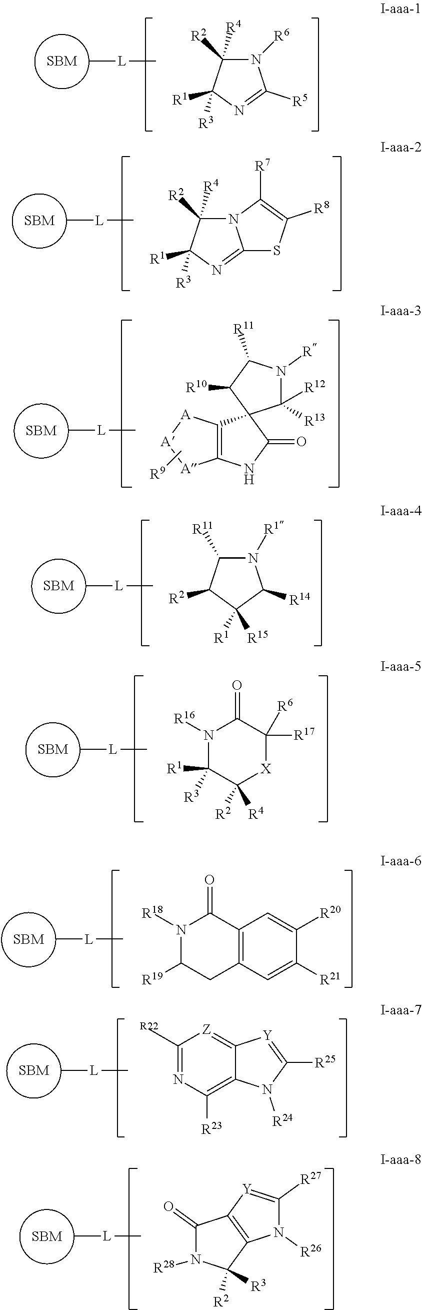

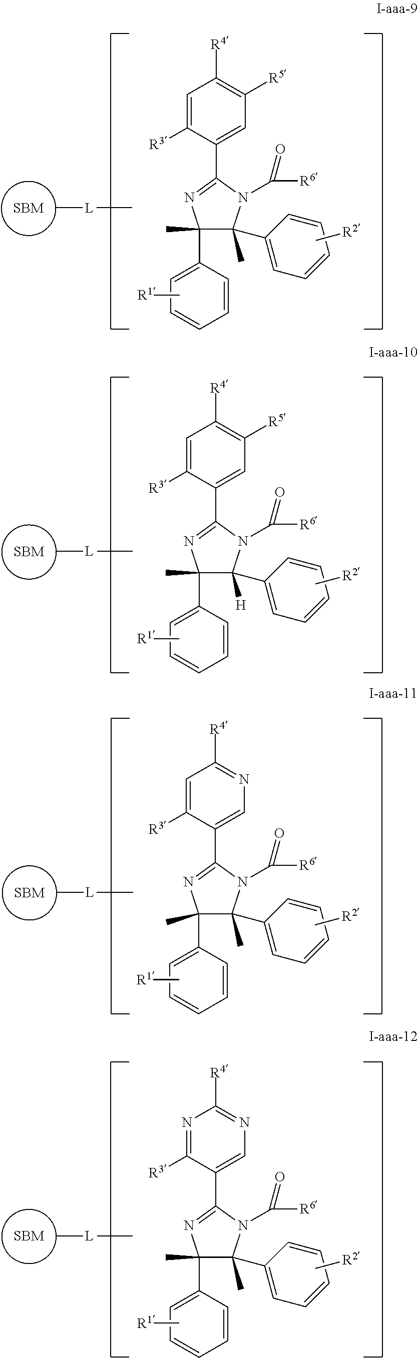

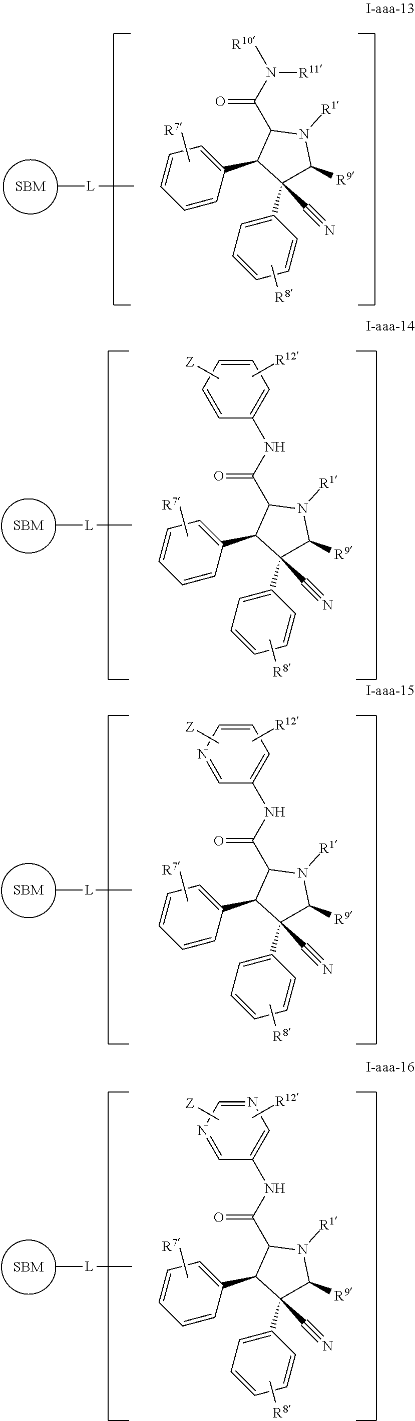

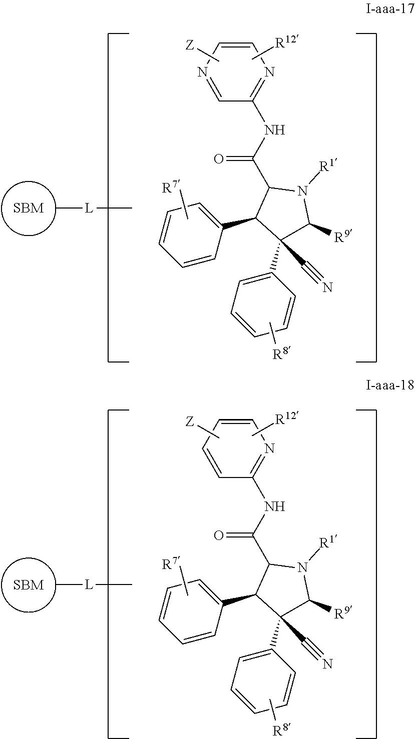

or a pharmaceutically acceptable salt thereof, wherein and SBM are as e me above and described in embodiments herein, and wherein:
X is selected from —CR₂—, —O—, —S—, —S(O)—, —S(O)₂—, and —NR—;
each R is independently hydrogen or an optionally substituted group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form a 4-7 membered saturated, partially unsaturated, or heteroaryl ring having 0-3 heteroatoms, in addition to the atom from which they are attached, independently selected from nitrogen, oxygen, and sulfur.
Y and Z are independently selected from —CR═ and —N═;
Ring W is fused ring selected from benzo and a 5-6 membered heteroaryl with 1-4 heteroatoms independently selected from nitrogen, oxygen, and sulfur;
R¹ and R² are independently an optionally substituted monocyclic or bicyclic ring selected from phenyl, a 5-10 membered aryl, and a 5-10 membered heteroaryl containing 1-4 heteroatoms independently selected from nitrogen, oxygen, and sulfur;
R³ and R⁴ are independently selected from hydrogen and C—₆ alkyl;
R⁵ is selected from an optionally substituted monocyclic or bicyclic ring selected from phenyl, a 5-10 membered aryl, and a 5-10 membered heteroaryl containing 1-4 heteroatoms independently selected from nitrogen, oxygen, and sulfur;
R⁶ is selected from hydrogen, —C(O)R, —C(O)OR, and —C(O)NR₂;
R⁷ is selected from hydrogen and R^{A};
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur:
R⁸ is selected from —C(O)R and R^{A}
R⁹ is a mono-, his-, or tri-substituent on Ring W, wherein each of the substituents are independently selected from halogen and an optionally substituted C₁₋₆ aliphatic;
R¹⁰ is selected from an optionally substituted monocyclic or bicyclic ring selected from phenyl, a 5-10 membered aryl, and a 5-10 membered heteroaryl containing 1-4 heteroatoms independently selected from nitrogen, oxygen, and sulfur;
R¹¹ is —C(O)OR or —C(O)NR₂;
R¹² and R¹³ are independently selected from hydrogen and R^{A}, or:
   R¹² and R¹³ are optionally taken together with their intervening atoms to form an optionally substituted 3-8 membered saturated, partially unsaturated, carbocyclic or heterocyclic ring having 1-3 heteroatoms independently selected from nitrogen, oxygen, and sulfur;
R¹⁴ is R^{A}.
R¹⁵ is —CN:
R¹⁶ is selected from R^{A}, —OR, —(CR₂)₀₋₆—C(O)R, —(CR₂)₀₋₆—C(O)OR, —(CR₂)₀₋₆—C(O)NR₂, —(CR₂)₀₋₆—S(O)₂R, —(CR₂)₀₋₆—N(R)S(O)₂R, —(CR₂)₀₋₆—S(O)₂NR₂;
R¹⁷ is selected from —(CR₂)₀₋₆—C(O)NR₂;
R¹⁸ and R¹⁹ are independently selected from hydrogen and R^{A};
R²⁰ and R²¹ are independently selected from hydrogen. R^{A}, halogen, and —OR, or:
   R²⁰ and R²¹ are optionally taken together with their intervening atoms to form a fused 5-7 membered partially unsaturated carbocyclic or heterocyclic ring having 1-3 heteroatoms independently selected from nitrogen, oxygen, and sulfur, or a fused 5-6 membered heteroaryl ring having 1-3 heteroatoms independently selected from nitrogen, oxygen, and sulfur;
R²², R²³, R²⁵ and R²⁷ are independently selected from hydrogen. R^{A}, halogen, —C(O)R, —C(O)OR, —C(O)NR₂, —NR₂, —OR, —S(O)R, —S(O)₂R, —S(O)₂NR₂;
R²⁴, R²⁶, and R²⁸ are independently selected from hydrogen, R^{A}, —C(O)R, —C(O)OR, —C(O)NR₂, —S(O)R, —S(O)₂R, and —S(O)₂NR₂;
R^{1′} and R^{2′} are independently selected from halogen, —C≡CR, —CN, —CF₃, and —NO₂;
R^{3′} is —OR:
R^{4′}, R^{5′}, R^{6′} are independently selected from hydrogen, halogen, R^{A}, —CN, —CF₃, —NR₂, —OR, —SR, and —S(O)₂R;
R^{7′} is a mono-, bis-, or tri-substituent, wherein each of the substituents are independently selected from halogen;
R^{8′} is a mono-, bis-, or tri-substituent, wherein each of the substituents are independently selected from hydrogen, halogen, R^{A}, —CN, —C≡CR, —NO₂, and —OR;
R^{9′} is R^{A};
Z¹ is selected from hydrogen, halogen, and —OR;
R^{10′} and R^{11′} are independently selected from hydrogen and R^{A};
R^{12′} is selected from —C(O)R, —C(O)OR, —C(O)NR₂, —OR, —S(O)₂R, —S(O)₂NR₂, and —S(O)R; and
R^{1″} is selected from hydrogen and R^{A}.

In certain embodiments, the present invention provides a compound of Formula I, wherein LBM is a MDM2 (i.e. human double minute 2 or HDM2) E3 ligase binding moiety thereby forming a compound of formula I-aaa-19, I-aaa-20, or I-aaa-21 respectively

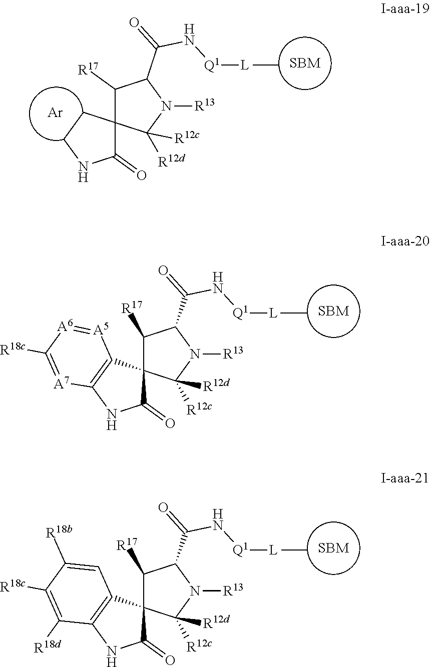

or a pharmaceutically acceptable salt thereof, wherein L and SBM are as defined above and described in embodiments herein, and wherein:
R^{1″} is selected from hydrogen and R^{A};
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
R¹⁰ is selected from an optionally substituted monocyclic or bicyclic ring selected from phenyl, a 5-10 membered aryl, and a 5-10 membered heteroaryl containing 1-4 heteroatoms independently selected from nitrogen, oxygen, and sulfur:
R₁₂ and R₁₃ are each independently selected from hydrogen and R^{A}, or:
   R¹² and R¹³ are optionally taken together with their intervening atoms to form an optionally substituted 4-8 membered saturated, partially unsaturated, carbocyclic or heterocyclic ring having 1-3 heteroatoms independently selected from nitrogen, oxygen, and sulfur;
A⁵ is selected from —C(R^{18a})═ and —N═;
A⁶ is selected from —C(R^{18b})═ and —N═:
A⁷ is selected from —C(R^{18d})═ and —N═;
R^{18a}, R^{18b}, R^{18c}, and R^{18d} are each independently selected from hydrogen, halogen, R^{A}, and —OR;
each R is independently hydrogen or an optionally substituted group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
Ring W is an optionally substituted fused ring selected from benzo and a 5-6 membered heteroaryl with 1-4 heteroatoms independently selected from nitrogen, oxygen, and sulfur; and
Q¹ is and optionally substituted bivalent group selected from alkylenyl, phenylenyl, heteroarylenyl, cycloalkylenyl, and heterocyclenyl.

In certain embodiments, the present invention provides a compound of Formula I, wherein LBM is an IAP E3 ubiquitin ligase binding moiety thereby forming a compound of formula I-bbb-1, I-bbb-2, I-bbb-3, or I-bbb-4 respectively:

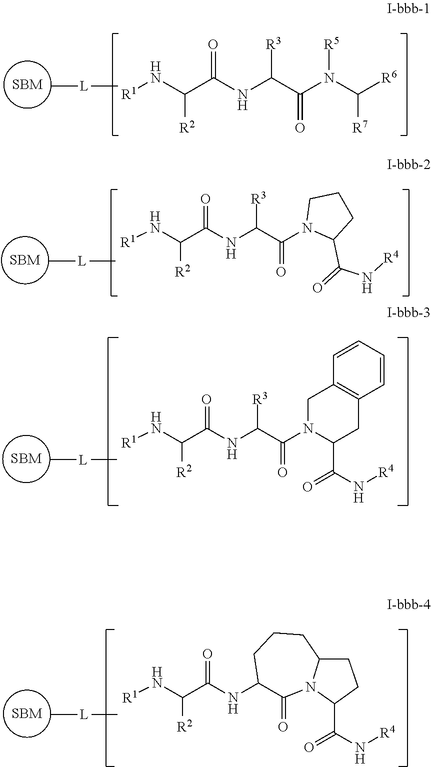

or a pharmaceutically acceptable salt thereof, wherein L and SBM are as defined above and described in embodiments herein, and wherein:
R¹ is selected from the group of H and alkyl;
R² is selected from the group of H and alkyl;
R³ is selected from the group of H, alkyl, cycloalkyl and heterocycloalkyl;
R⁴ is selected from alkyl, cycloalkyl, heterocycloalkyl, cycloalkylalkyl, heterocycloalkylalkyl, aryl, arylalkyl, heteroaryl, heteroarylalkyl, further optionally substituted with 1-3 substituents selected from halogen, alkyl, haloalkyl, hydroxyl, alkoxy, cyano, (hetero)cycloalkyl or (hetero)aryl, or —C(O)NH—R⁴, where R⁴ is selected from alkyl, cycloalkyl, heterocycloalkyl, cycloalkylalkyl, heterocycloalkylalkyl, aryl, arylalkyl, heteroaryl, heteroarylalkyl, further optionally substituted with 1-3 substituents as described above;
R⁵ and R⁶ are independently selected from the group of H, alkyl, cycloalkyl, heterocycloalkyl, aryl, heteroaryl or fused rings; and
R⁷ is selected from the group of cycloalkyl, cycloalkylalkyl, heterocycloalkyl, heterocycloalkylalkyl, aryl, arylalkyl, heteroaryl or heteroarylalkyl, each one further optionally substituted with 1-3 substituents selected from halogen, alkyl, haloalkyl, hydroxyl, alkoxy, cyano, (hetero)cycloalkyl or (hetero)aryl, or —C(O)NH—R⁴, where R⁴ is selected from alkyl, cycloalkyl, heterocycloalkyl, cycloalkylalkyl, heterocycloalkylalkyl, aryl, arylalkyl, heteroaryl, heteroarylalkyl, further optionally substituted with 1-3 substituents as described above,
as defined and described in WO 2017/011590 and US 2017/0037004, the entirety of each of which is herein incorporated by reference.

In certain embodiments, the present invention provides a compound of Formula I, wherein LBM is an E3 ubiquitin ligase (cereblon) binding moiety, a DCAF15 E3 ubiquitin ligase binding moiety, or a VHL E3 ubiquitin ligase binding moiety; thereby forming a compound of formula I-ccc-1, I-ccc-2, or I-ccc-3:

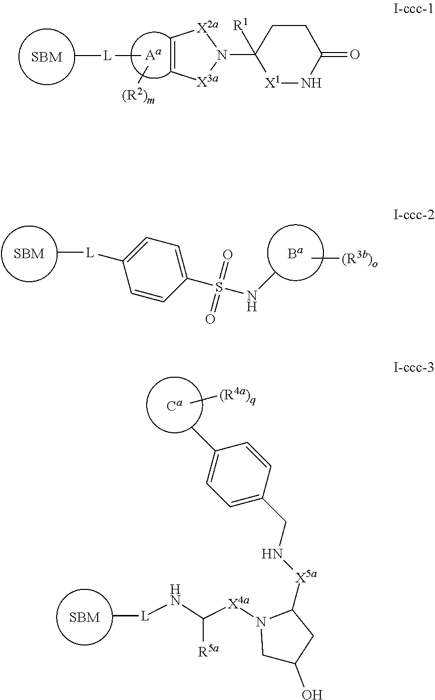

or a pharmaceutically acceptable salt thereof, wherein L and SBM is as defined above and described in embodiments herein, and wherein:
each of X¹, X^{2a}, and X^{3a} is independently a bivalent moiety selected from a covalent bond, —CH₂—, —C(O)—, —C(S)—,

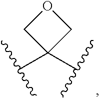 [SMILES: *C1(*)COC1]

—CR₂CR₂, —N═CR—, or —CR═CR—;
each of X^{4a} and X^{5a} is independently a bivalent moiety selected from —CH₂—, —C(O)—, —C(S)—, or

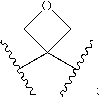 [SMILES: *C1(*)COC1]

R¹ is hydrogen, deuterium, halogen, —CN, —OR, —SR, —S(O)R, —S(O)₂R, —NR₂, or an optionally substituted C₁₋₄ aliphatic:
each of R², R^{3b}, and R^{4a} is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
R^{5a} is hydrogen or C₁₋₆ aliphatic:
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur:
Ring A^{a} is a fused ring selected from 6-membered aryl containing 0-2 nitrogen atoms, 5 to 7-membered partially saturated carbocyclyl, 5 to 7-membered partially saturated heterocyclyl with 1-2 heteroatoms independently selected from nitrogen, oxygen or sulfur, or 5-membered heteroaryl with 1-3 heteroatoms independently selected from nitrogen, oxygen or sulfur;
Ring B^{a} is selected from 6-membered aryl containing 0-2 nitrogen atoms or a 8-10 membered bicyclic heteroaryl having 1-5 heteroatoms independently selected from nitrogen, oxygen, or sulfur;
Ring C^{a} is a selected from 6-membered aryl containing 0-2 nitrogen atoms or a 5-membered heteroaryl with 1-3 heteroatoms independently selected from nitrogen, oxygen or sulfur;
m is 0, 1, 2, 3 or 4;
is 0, 1, 2, 3 or 4;
q is 0, 1, 2, 3 or 4; and
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In certain embodiments, the present invention provides a compound of Formula I-ccc-1, wherein LBM is an E3 ubiquitin ligase (cereblon) binding moiety thereby forming a compound of formula I-ccc′-1 or I-ccc″-1:

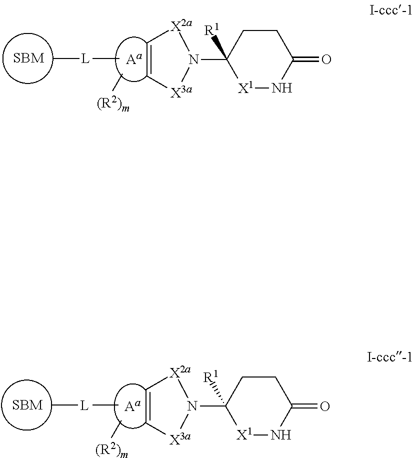

or a pharmaceutically acceptable salt thereof, wherein SBM, L, Ring A^{a}, X¹, X^{2a}, X^{3a}, R¹, R² and in are as described above.

In certain embodiments, the present invention provides a compound of Formula I, wherein LBM is an E3 ubiquitin ligase (cereblon) binding moiety, a DCAF15 E3 ubiquitin ligase binding moiety, or a VHL E3 ubiquitin ligase binding moiety; thereby forming a compound of formula I-ccc-1′, I-ccc-2′, or I-ccc-3′:

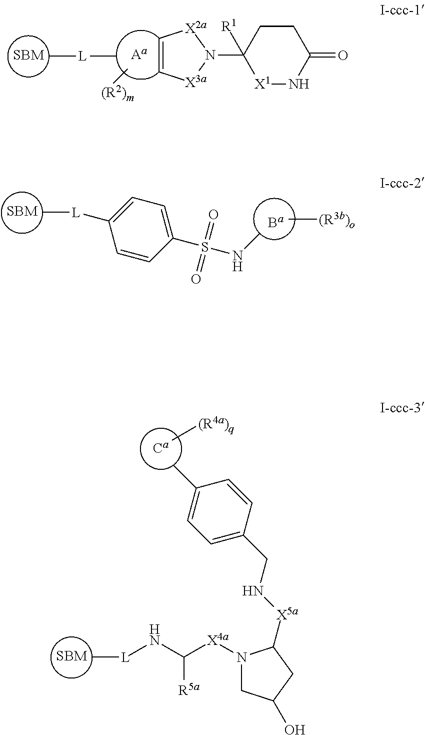

or a pharmaceutically acceptable salt thereof, wherein L and SBM is as defined above and described in embodiments herein, and wherein:
each of X¹, X^{2a}, and X^{3a} is independently a bivalent moiety selected from a covalent bond, —CH₂—, —C(O)—, —C(S)—,

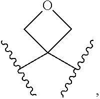 [SMILES: *C1(*)COC1]

—CR₂CR₂, —N═CR—, or —CR═CR—;
each of X^{4a} and X^{5a} is independently a bivalent moiety selected from —CH₂—, —C(O)—, —C(S)—, or

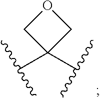 [SMILES: *C1(*)COC1]

R¹ is hydrogen, deuterium, halogen, —CN, —OR, —SR, —S(O)R, —S(O)₂R, —NR₂, or an optionally substituted C₁₋₄ aliphatic;
each of R², R^{3b}, and R^{4a} is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R; or:
   two R² groups of Ring Aa are taken together with their intervening atoms to form an optionally substituted ring selected from a 3-10 membered saturated or partially unsaturated carbocyclyl or heterocyclyl having 1-4 heteroatoms independently selected from nitrogen, oxygen, or sulfur; benzo; or a 5-10 membered heteroaryl having 1-4 heteroatoms independently selected from nitrogen, oxygen, or sulfur;
R^{5a} is hydrogen or C₁₋₆ aliphatic;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, a 5-6 membered heteroaryl ring having 1-4 heteroatoms independently selected from nitrogen, oxygen, and sulfur, an 8-15 membered saturated or partially unsaturated tricyclic heterocyclylenyl having 1-5 heteroatoms independently selected from nitrogen, oxygen, and sulfur, or an 8-15 membered tricyclic heteroarylenyl having 1-5 heteroatoms independently selected from nitrogen, oxygen, and sulfur:
Ring A^{a} is a fused ring selected from 6-membered aryl containing 0-2 nitrogen atoms, 5 to 7-membered partially saturated carbocyclyl, 5- to 7-membered partially saturated heterocyclyl with 1-2 heteroatoms independently selected from nitrogen, oxygen or sulfur, or 5-membered heteroaryl with 1-3 heteroatoms independently selected from nitrogen, oxygen or sulfur;
Ring B^{a} is selected from 6-membered aryl containing 0-2 nitrogen atoms or a 8-10 membered bicyclic heteroaryl having 1-5 heteroatoms independently selected from nitrogen, oxygen, or sulfur;
Ring C^{a} is a selected from 6-membered aryl containing 0-2 nitrogen atoms or a 5-membered heteroaryl with 1-3 heteroatoms independently selected from nitrogen, oxygen or sulfur;
m is 0, 1, 2, 3 or 4:
is 0, 1, 2, 3 or 4;
q is 0, 1, 2, 3 or 4; and
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In certain embodiments, the present invention provides a compound of Formula I-ccc-1′, wherein LBM is an E3 ubiquitin ligase (cereblon) binding moiety thereby forming a compound of formula I-ccc′-1′ or I-ccc″-1′:

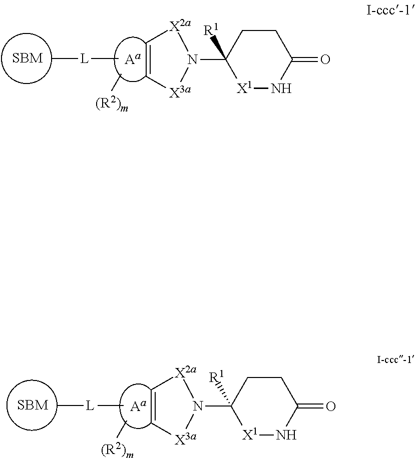

or a pharmaceutically acceptable salt thereof, wherein SBM, L, Ring A^{a}, X¹, X^{2a}, X^{3a}, R¹, R² and in are as described above.

In some embodiments, LBM is of formulae I-ccc-A, I-ccc-B, or I-ccc-C:

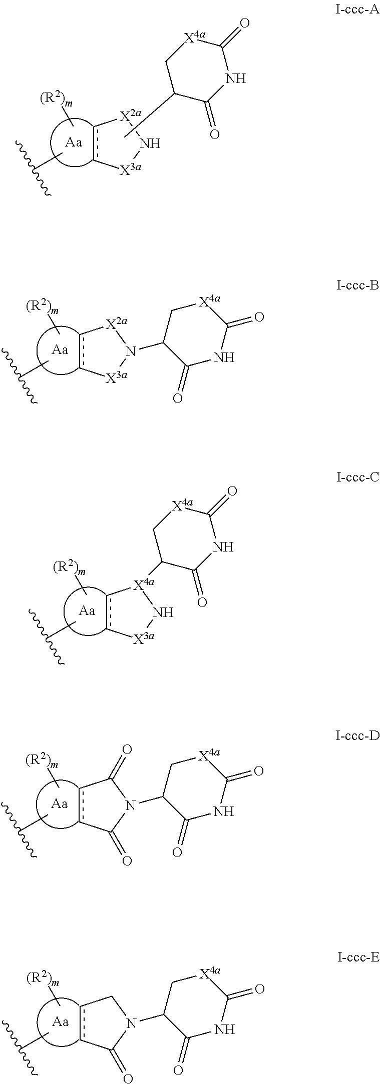

or a pharmaceutically acceptable salt thereof, wherein Ring A^{a}, X^{2a}, X^{3a}, R¹, R² and m are as defined above and described above; and:
X^{4a} is CH, CR, or N.

As defined above and described herein, each of X¹, X^{2a}, and X^{3a} is independently a bivalent moiety selected from a covalent bond, —CH₂—, —C(O)—, —C(S)—,

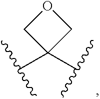 [SMILES: *C1(*)COC1]

—CR₂CR₂, —N═CR—, or —CR═CR—.

In some embodiments, X¹ is a covalent bond, —CH₂—, —C(O)—, —C(S)—,

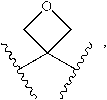 [SMILES: *C1(*)COC1]

—CR₂CR₂, —N═CR—, or —CR═CR—.

In some embodiments, X¹ is selected from those depicted in Table 1, below.

In some embodiments, X^{2a} is a covalent bond, —CH₂—, —C(O)—, —C(S)—,

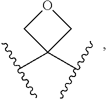 [SMILES: *C1(*)COC1]

—CR₂CR₂, —N═CR—, or —CR═CR—. In some embodiments, X^{2a} is —C(O)—.

In some embodiments, X^{2a} is selected from those depicted in Table 1, below.

In some embodiments, X^{3a} is a covalent bond, —CH₂—, —C(O)—, —C(S)—,

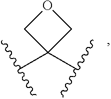 [SMILES: *C1(*)COC1]

—CR₂CR₂, —N═CR—, or —CR═CR—. In some embodiments, X^{3a} is —C(O)—.

In some embodiments, X^{2a} and X^{3a} are —C(O)—. In some embodiments, X^{2a} is —C(O)—; and X^{3a} is —CH₂—.

In some embodiments, X^{3a} is selected from those depicted in Table 1, below.

As defined above and described herein, X^{4a} is CH. CR, or N. In some embodiments, X^{4a} is CH. In some embodiments, X^{4a} is CR. In some embodiments, X^{4a} is N.

As defined above and described herein, each of X⁴ and X⁵ is independently a bivalent moiety selected from —CH₂—, —C(O)—, —C(S)—, or

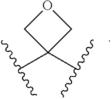 [SMILES: *C1(*)COC1]

In some embodiments, X^{4a} is —CH₂—, —C(O)—, —C(S)—, or

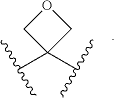 [SMILES: *C1(*)COC1]

In some embodiments, X^{4a} is selected from those depicted in Table 1, below.

In some embodiments, X^{5a} is —CH₂—, —C(O)—, —C(S)—, or

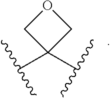 [SMILES: *C1(*)COC1]

In some embodiments, X^{5a} is selected from those depicted in Table 1, below.

As defined above and described herein, R¹ is hydrogen, deuterium, halogen, —CN, —OR, —SR, —S(O)R, —S(O)₂R, —NR₂, or an optionally substituted C₁₋₄ aliphatic.

In some embodiments, R¹ is hydrogen, deuterium, halogen, —CN, —OR, —SR, —S(O)R, —S(O)₂R, —NR₂, or an optionally substituted C₁₋₄ aliphatic.

In some embodiments, R¹ is selected from those depicted in Table 1, below.

As defined above and described herein, each of R², R^{3b}, and R^{4a} is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R.

In some embodiments, R² is hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R.

In some embodiments, two R² groups of Ring A^{a} are taken together with their intervening atoms to form an optionally substituted ring selected from a 3-10 membered saturated or partially unsaturated carbocyclyl or heterocyclyl having 1-4 heteroatoms independently selected from nitrogen, oxygen, or sulfur; benzo; or a 5-10 membered heteroaryl having 1-4 heteroatoms independently selected from nitrogen, oxygen, or sulfur.

In some embodiments, two R² groups of Ring A^{a} are taken together with their intervening atoms to form an optionally substituted ring selected from a 3-10 membered saturated or partially unsaturated carbocyclyl. In some embodiments, two R² groups of Ring A^{a} are taken together with their intervening atoms to form an optionally substituted ring selected from a 5-6 membered saturated or partially unsaturated carbocyclyl.

In some embodiments, two R² groups of Ring A^{a} are taken together with their intervening atoms to form an optionally substituted ring selected from a 3-10 membered saturated or partially unsaturated heterocyclyl having 1-4 heteroatoms independently selected from nitrogen, oxygen, or sulfur. In some embodiments, two R² groups of Ring A^{a} are taken together with their intervening atoms to form an optionally substituted ring selected from a 4-7 membered saturated or partially unsaturated heterocyclyl having 1-4 heteroatoms independently selected from nitrogen, oxygen, or sulfur. In some embodiments, two R² groups of Ring A^{a} are taken together with their intervening atoms to form an optionally substituted ring selected from a 5-6 membered saturated or partially unsaturated heterocyclyl having 1-4 heteroatoms independently selected from nitrogen, oxygen, or sulfur.

In some embodiments, two R² groups of Ring A^{a} are taken together with their intervening atoms to form an optionally substituted benzo.

In some embodiments, two R² groups of Ring A^{a} are taken together with their intervening atoms to form an optionally substituted ring selected from a 5-10 membered heteroaryl having 1-4 heteroatoms independently selected from nitrogen, oxygen, or sulfur. In some embodiments, two R² groups of Ring A^{a} are taken together with their intervening atoms to form an optionally substituted ring selected from a 5-6 membered heteroaryl having 1-4 heteroatoms independently selected from nitrogen, oxygen, or sulfur.

In some embodiments, R² is selected from those depicted in Table 1, below.

In some embodiments, R^{3b} is hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R.

In some embodiments, R^{3b} is methyl.

In some embodiments, R^{3b} is selected from those depicted in Table 1, below.

In some embodiments, R^{4a} is hydrogen, —R⁶, halogen, —CN, —NO₂, —OR, —SR, —NR₂, —S(O)₂R, —S(O)₂N_{R}—S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R.

In some embodiments, R^{4a} is methyl.

In some embodiments, R^{4a} is selected from those depicted in Table 1, below.

As defined above and described herein, R^{a} is hydrogen or C₁₋₆ aliphatic.

In some embodiments, R^{5a} is t-butyl.

In some embodiments, R^{5a} is selected from those depicted in Table 1, below.

As defined above and described herein,

As defined above and described herein, each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, a 5-6 membered heteroaryl ring having 1-4 heteroatoms independently selected from nitrogen, oxygen, and sulfur, an 8-15 membered saturated or partially unsaturated tricyclic heterocyclylenyl having 1-5 heteroatoms independently selected from nitrogen, oxygen, and sulfur, or an 8-15 membered tricyclic heteroarylenyl having 1-5 heteroatoms independently selected from nitrogen, oxygen, and sulfur. In some embodiments, each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, R⁶ is an optionally substituted C₁₋₆ aliphatic group. In some embodiments, R⁶ is an optionally substituted phenyl. In some embodiments, R⁶ is an optionally substituted 4-7 membered saturated or partially unsaturated heterocyclic ring having 1-2 heteroatoms independently selected from nitrogen, oxygen, and sulfur. In some embodiments, R⁶ is an optionally substituted 5-6 membered heteroaryl ring having 1-4 heteroatoms independently selected from nitrogen, oxygen, and sulfur.

In some embodiments, R⁶ is an optionally substituted 8-15 membered saturated or partially unsaturated tricyclic heterocyclylenyl having 1-5 heteroatoms independently selected from nitrogen, oxygen, and sulfur.

In some embodiments, R⁶ is a 12-membered saturated or partially unsaturated tricyclic heterocyclylenyl having 1-5 heteroatoms independently selected from nitrogen, oxygen, and sulfur. In some embodiments, R⁶ is a 12-membered saturated or partially unsaturated tricyclic heterocyclylenyl having 1-5 nitrogen heteroatoms. In some embodiments, R⁶ is a 12-membered saturated or partially unsaturated tricyclic heterocyclylenyl having 3 nitrogen heteroatoms.

In some embodiments, R⁶ is a 5,6,5-fused saturated or partially unsaturated tricyclic heterocyclylenyl having 1-5 heteroatoms independently selected from nitrogen, oxygen, and sulfur. In some embodiments, R⁶ is a 5,6,5-fused membered saturated or partially unsaturated tricyclic heterocyclylenyl having 1-5 nitrogen heteroatoms. In some embodiments, R⁶ is a 5,6,5-fused membered saturated or partially unsaturated tricyclic heterocyclylenyl having 3 nitrogen heteroatoms.

In some embodiments, R⁶ is a 5,5,6-fused saturated or partially unsaturated tricyclic heterocyclylenyl having 1-5 heteroatoms independently selected from nitrogen, oxygen, and sulfur. In some embodiments, R⁶ is a 5,5,6-fused membered saturated or partially unsaturated tricyclic heterocyclylenyl having 1-5 nitrogen heteroatoms. In some embodiments. R⁶ is a 5,5,6-fused membered saturated or partially unsaturated tricyclic heterocyclylenyl having 3 nitrogen heteroatoms.

In some embodiments, R⁶ is a 13-membered saturated or partially unsaturated tricyclic heterocyclylenyl having 1-5 heteroatoms independently selected from nitrogen, oxygen, and sulfur. In some embodiments. R⁶ is a 13-membered saturated or partially unsaturated tricyclic heterocyclylenyl having 1-5 nitrogen heteroatoms. In some embodiments. R⁶ is a 13-membered saturated or partially unsaturated tricyclic heterocyclylenyl having 3 nitrogen heteroatoms.

In some embodiments, R⁶ is a 5,6,6-fused saturated or partially unsaturated tricyclic heterocyclylenyl having 1-5 heteroatoms independently selected from nitrogen, oxygen, and sulfur. In some embodiments, R⁶ is a 5,6,6-fused membered saturated or partially unsaturated tricyclic heterocyclylenyl having 1-5 nitrogen heteroatoms. In some embodiments, R⁶ is a 5,6,6-fused membered saturated or partially unsaturated tricyclic heterocyclylenyl having 3 nitrogen heteroatoms.

In some embodiments, R⁶ is a 6,5,6-fused saturated or partially unsaturated tricyclic heterocyclylenyl having 1-5 heteroatoms independently selected from nitrogen, oxygen, and sulfur. In some embodiments, R⁶ is a 6,5,6-fused membered saturated or partially unsaturated tricyclic heterocyclylenyl having 1-5 nitrogen heteroatoms. In some embodiments, R⁶ is a 6,5,6-fused membered saturated or partially unsaturated tricyclic heterocyclylenyl having 3 nitrogen heteroatoms.

In some embodiments, R⁶ is a 14-membered saturated or partially unsaturated tricyclic heterocyclylenyl having 1-5 heteroatoms independently selected from nitrogen, oxygen, and sulfur.

In some embodiments. R⁶ is or an 10-15 membered tricyclic heteroarylenyl having 1-5 heteroatoms independently selected from nitrogen, oxygen, and sulfur.

In some embodiments, R⁶ is a 12-membered saturated or partially unsaturated tricyclic heteroarylenyl having 1-5 heteroatoms independently selected from nitrogen, oxygen, and sulfur. In some embodiments, R⁶ is a 12-membered saturated or partially unsaturated tricyclic heteroarylenyl having 1-5 nitrogen heteroatoms. In some embodiments, R⁶ is a 12-membered saturated or partially unsaturated tricyclic heteroarylenyl having 3 nitrogen heteroatoms.

In some embodiments, R⁶ is a 5,6,5-fused saturated or partially unsaturated tricyclic heteroarylenyl having 1-5 heteroatoms independently selected from nitrogen, oxygen, and sulfur. In some embodiments, R⁶ is a 5,6,5-fused membered saturated or partially unsaturated tricyclic heteroarylenyl having 1-5 nitrogen heteroatoms. In some embodiments, R⁶ is a 5,6,5-fused membered saturated or partially unsaturated tricyclic heteroarylenyl having 3 nitrogen heteroatoms.

In some embodiments, R⁶ is a 5,5,6-fused saturated or partially unsaturated tricyclic heteroarylenyl having 1-5 heteroatoms independently selected from nitrogen, oxygen, and sulfur. In some embodiments, R⁶ is a 5,5,6-fused membered saturated or partially unsaturated tricyclic heteroarylenyl having 1-5 nitrogen heteroatoms. In some embodiments, R⁶ is a 5,5,6-fused membered saturated or partially unsaturated tricyclic heteroarylenyl having 3 nitrogen heteroatoms.

In some embodiments. R⁶ is a 13-membered saturated or partially unsaturated tricyclic heteroarylenyl having 1-5 heteroatoms independently selected from nitrogen, oxygen, and sulfur. In some embodiments, R⁶ is a 13-membered saturated or partially unsaturated tricyclic heteroarylenyl having 1-5 nitrogen heteroatoms. In some embodiments, R⁶ is a 13-membered saturated or partially unsaturated tricyclic heteroarylenyl having 3 nitrogen heteroatoms.

In some embodiments, R⁶ is a 5,6,6-fused saturated or partially unsaturated tricyclic heteroarylenyl having 1-5 heteroatoms independently selected from nitrogen, oxygen, and sulfur. In some embodiments, R⁶ is a 5,6,6-fused membered saturated or partially unsaturated tricyclic heteroarylenyl having 1-5 nitrogen heteroatoms. In some embodiments, R⁶ is a 5,6,6-fused membered saturated or partially unsaturated tricyclic heteroarylenyl having 3 nitrogen heteroatoms.

In some embodiments, R⁶ is a 6,5,6-fused saturated or partially unsaturated tricyclic heteroarylenyl having 1-5 heteroatoms independently selected from nitrogen, oxygen, and sulfur. In some embodiments, R⁶ is a 6,5,6-fused membered saturated or partially unsaturated tricyclic heteroarylenyl having 1-5 nitrogen heteroatoms. In some embodiments, R⁶ is a 6,5,6-fused membered saturated or partially unsaturated tricyclic heteroarylenyl having 3 nitrogen heteroatoms.

In some embodiments, R⁶ is selected from those depicted in Table 1, below.

As defined above and described herein, Ring A^{a} is a fused ring selected from 6-membered aryl containing 0-2 nitrogen atoms, 5 to 7-membered partially saturated carbocyclyl, 5 to 7-membered partially saturated heterocyclyl with 1-2 heteroatoms independently selected from nitrogen, oxygen or sulfur, or 5-membered heteroaryl with 1-3 heteroatoms independently selected from nitrogen, oxygen or sulfur.

In some embodiments Ring A^{a} is a fused 6-membered aryl containing 0-2 nitrogen atoms. In some embodiments Ring A^{a} is a fused 5 to 7-membered partially saturated carbocyclyl. In some embodiments Ring A^{a} is a fused 5 to 7-membered partially saturated heterocyclyl with 1-2 heteroatoms independently selected from nitrogen, oxygen or sulfur. In some embodiments Ring A^{a} is a fused 5-membered heteroaryl with 1-3 heteroatoms independently selected from nitrogen, oxygen or sulfur.

In some embodiments, Ring A^{a} is a fused phenyl.

In some embodiments, Ring A^{a} is selected from those depicted in Table 1, below.

As defined above and described herein, Ring B^{a} is selected from 6-membered aryl containing 0-2 nitrogen atoms or a 8-10 membered bicyclic heteroaryl having 1-5 heteroatoms independently selected from nitrogen, oxygen, or sulfur.

In some embodiments, Ring B^{a} is a 6-membered aryl containing 0-2 nitrogen atoms. In some embodiments, Ring B^{a} is a 8-10 membered bicyclic heteroaryl having 1-5 heteroatoms independently selected from nitrogen, oxygen, or sulfur.

In some embodiments, Ring B^{a} is

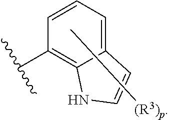

In some embodiments, Ring B^{a} is selected from those depicted in Table 1, below.

As defined above and described herein. Ring C^{a} is selected from 6-membered aryl containing 0-2 nitrogen atoms or a 5-membered heteroaryl with 1-3 heteroatoms independently selected from nitrogen, oxygen or sulfur.

In some embodiments, Ring C^{a} is a 6-membered aryl containing 0-2 nitrogen atoms. In some embodiments, Ring C^{a} is a 5-membered heteroaryl with 1-3 heteroatoms independently selected from nitrogen, oxygen or sulfur.

In some embodiments, Ring C^{a} is

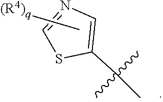

In some embodiments, Ring C^{a} is selected from those depicted in Table 1, below.

As defined above and described herein, m is 0, 1, 2, 3 or 4.

In some embodiments, m is 0, 1, 2, 3 or 4.

In some embodiments, m is 1. In some embodiments, m is 2. In some embodiments, m is 3. In some embodiments, m is 4.

In some embodiments, m is selected from those depicted in Table 1, below.

In some embodiments, o is selected from those depicted in Table 1, below.

As defined above and described herein, o is 0, 1, 2, 3 or 4.

In some embodiments, o is 3. In some embodiments, o is 1. In some embodiments, o is 2. In some embodiments, o is 3. In some embodiments, o is 4.

In some embodiments, o is selected from those depicted in Table 1, below.

As defined above and described herein, q is 0, 1, 2, 3 or 4.

In some embodiments, q is 3. In some embodiments, q is 1. In some embodiments, q is 2. In some embodiments, q is 3. In some embodiments, q is 4.

In some embodiments, q is selected from those depicted in Table 1, below.

As defined above and described herein, each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or: two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, R is hydrogen. In some embodiments, R is phenyl. In some embodiments, R is a 4-7 membered saturated or partially unsaturated heterocyclic having 1-2 heteroatoms independently selected from nitrogen, oxygen, and sulfur. In some embodiments, R is a 5-6 membered heteroaryl ring having 1-4 heteroatoms independently selected from nitrogen, oxygen, and sulfur. In some embodiments, two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, R is selected from those depicted in Table 1, below.

In certain embodiments, the present invention provides a compound of formula I, wherein LBM is a VHL E3 ubiquitin ligase binding moiety, thereby forming a compound of formula I-ddd:

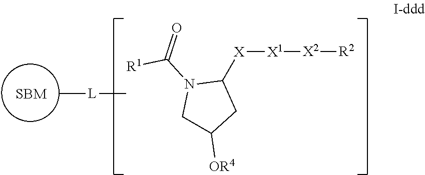

or a pharmaceutically acceptable salt thereof, wherein L and SBM is as defined above and described in embodiments herein, and wherein:
X is —C(O)—, —C(O)NR—, —SO₂—, —SO₂NR—, or an optionally substituted 5-membered heterocyclic ring:
X¹ is a bivalent group selected from a covalent bond, —O—, —C(O)—, —C(S)—, —C(R)₂—, —NR—, —S(O)—, or —SO₂—;
X² is an optionally substituted bivalent group selected from C₁₋₆ saturated or unsaturated alkylene, phenylenyl, a 5-6 membered heteroarylenyl containing 1-4 heteroatoms independently selected from nitrogen, oxygen, and sulfur, or a 4-11 membered saturated or partially unsaturated monocyclic, bicyclic, bridged bicyclic, or spirocyclic carbocyclylenyl or heterocyclylenyl with 1-3 heteroatoms independently selected from nitrogen, oxygen, and sulfur;
R¹ is R^{A}, —C(R)₂R^{A}, —OR, —SR, —N(R)₂, —C(R)₂OR, —C(R)₂N(R)₂, —C(R)₂NRC(O)R, —C(R)₂NRC(O)N(R)₂, —NRC(O)OR, —NRC(O)R, —NRC(O)N(R)₂, or —NRSO₂R;
each R is independently hydrogen, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to fomi an optionally substituted 3-11 membered saturated or partially unsaturated monocyclic, bicyclic, bridged bicyclic, or spirocyclic carbocyclic ring or heterocyclic ring with 1-4 heteroatoms independently selected from nitrogen, oxygen, and sulfur;
R^{A} is an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
R² is hydrogen, halogen, —CN,

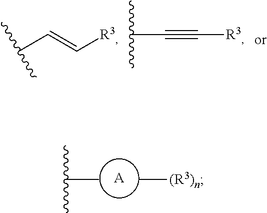

Ring A is a ring selected from phenyl, a 5-6 membered heteroaryl containing 1-4 heteroatoms independently selected from nitrogen, oxygen, and sulfur, or a 4 to 9-membered saturated or partially unsaturated monocyclic, bicyclic, bridged bicyclic, or spirocyclic carbocyclyl or heterocyclyl with 1-3 heteroatoms independently selected from nitrogen, oxygen, and sulfur;
each of R³ is independently hydrogen, halogen, C₁₋₆alkyl, C₁₋₆haloalkyl, —CN, —NO₂, —OR, —SR, —N(R)₂, —Si(R)₃, —SO₂R, —SO₂N(R)₂, —S(O)R, —C(O)R, —C(O)OR, —C(O)N(R)₂, —C(O)N(R)OR, —C(R)₂NRC(O)R, —C(R)₂NRC(O)N(R)₂, —OC(O)R, —OC(O)N(R)₂, —OP(O)(R)₂, —OP(O)(OR)₂, —OP(O)(OR)N(R)₂, —OP(O)(N(R)₂)₂—, —N(R)C(O)OR, —N(R)C(O)R, —NRC(O)N(R)₂, —N(R)SO₂R, —NP(O)(R)₂, —N(R)P(O)(OR)₂, —N(R)P(O)(OR)N(R)₂, —N(R)P(O)(N(R)₂)₂, —N(R)SO₂R, or R^{A}; or
   two R³ groups are optionally taken together to form an optionally substituted 5-7 membered partially unsaturated or aryl fused ring having 0-2 heteroatoms independently selected from nitrogen, oxygen, and sulfur;
R⁴ is hydrogen, —C(O)R, —C(O)OR, —C(O)NR₂, —P(O)R₂, —P(O)(OR)₂, —(CR₂)₁₋₃OP(O)R₂, —(CR₂)₁₋₃OP(O)(OR)₂, or R^{A}:
n is 0, 1, 2, 4, or 5.

In certain embodiments, the present invention provides a compound of formula I, wherein LBM is an IAP binding moiety thereby forming a compound of formula I-fff:

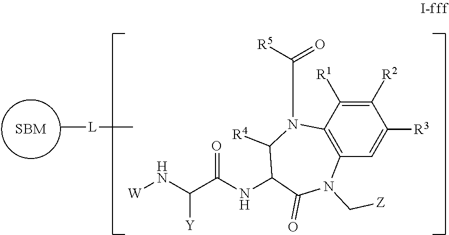

or a pharmaceutically acceptable salt thereof, wherein L and SBM are as defined above and described in embodiments herein, and wherein:
W is selected from H and lower alkyl that optionally may be substituted with 1-3 deuterium atoms;
Y is lower alkyl that optionally may be substituted with OR⁶;
R¹, R² and R³ are the same or different and each is independently selected from H and cyano; R⁴ is lower alkyl;
R⁵ is selected from the group a) lower alkyl that optionally may be substituted with SO₂R⁶ and OR⁶, b) heterocyclyl, and c) aryl that optionally may be substituted with C(O)R⁷, halo and cyano;
Z is selected from the group a) aryl that optionally may be substituted with lower alkyl, OR⁶, halogen and aryl that optionally may be substituted with halogen, b) heteroaryl that optionally may be substituted with lower alkyl, cycloalkyl, OR⁶, halogen, oxo and aryl that optionally may substituted with cyano, and c) aryl fused with heterocyclyl, wherein the aryl optionally may be substituted with OR⁶ and halogen, and the heterocyclyl optionally may be substituted with oxo, and d) heterocyclyl;
R⁶ is selected from H and lower alkyl that optionally may be substituted with halogen and deuterium; and
R⁷ is lower alkyl,
as described and defined in WO 2014/044622, US 2015/0225449. WO 2015/071393, and US 2016/0272596, the entirety of each of which is herein incorporated by reference.

In certain embodiments, the present invention provides a compound of formula I, wherein LBM is a MDM2 binding moiety thereby forming a compound of formula I-ggg:

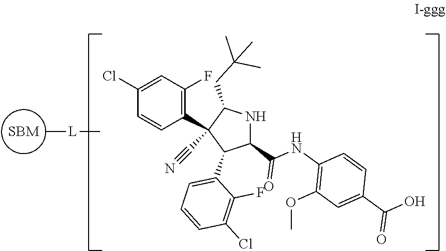

or a pharmaceutically acceptable salt thereof, wherein L and SBM are as defined above and described in embodiments herein, as described and defined in Hines. J. et al., Cancer Res. (DOI: 10.1158/0008-5472.CAN-18-2918), the entirety of each of which is herein incorporated by reference.

In certain embodiments, the present invention provides a compound of formula I, wherein LBM is a DCAF16 binding moiety thereby forming a compound of formula I-hhh:

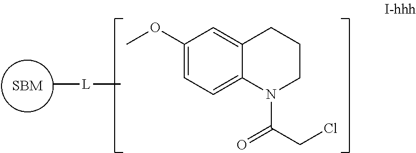

or a pharmaceutically acceptable salt thereof, wherein L and SBM are as defined above and described in embodiments herein, as described and defined in Zhang, X. et al., bioRxiv (doi: https://doi.org/10.1101/443804), the entirety of each of which is herein incorporated by reference.

In certain embodiments, the present invention provides a compound of formula I, wherein LBM is a RNF114 binding moiety thereby forming a compound of formula I-iii:

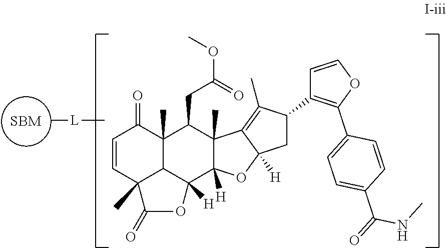

or a pharmaceutically acceptable salt thereof, wherein L and SBM are as defined above and described in embodiments herein, as described and defined in Spradin, J. N. et al., bioRxiv (doi: https://doi.org/10.1101/436998), the entirety of each of which is herein incorporated by reference.

In certain embodiments, the present invention provides a compound of formula I, wherein LBM is a RNF4 binding moiety thereby forming a compound of formula I-jjj:

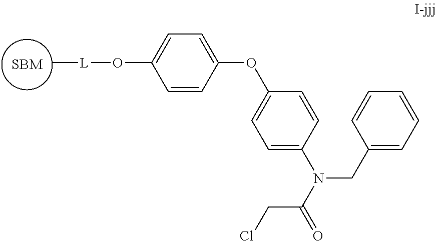

or a pharmaceutically acceptable salt thereof, wherein L and SBM are as defined above and described in embodiments herein, as described and defined in Ward, C. C., et al., bioRxiv (doi: https.//doi.org/10.1101/439125), the entirety of each of which is herein incorporated by reference.

In some embodiments, LBM is

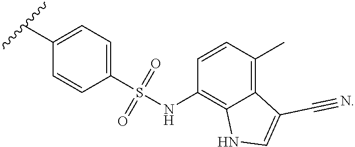 [SMILES: *c1ccc(S(=O)(=O)Nc2ccc(C)c3c(C#N)c[nH]c23)cc1]

In some embodiments, LBM is

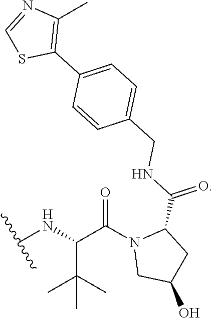 [SMILES: *N[C@H](C(=O)N1C[C@H](O)C[C@H]1C(=O)NCc1ccc(-c2scnc2C)cc1)C(C)(C)C]

In some embodiments, LBM is

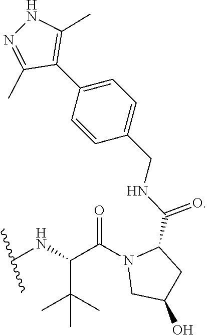 [SMILES: *N[C@H](C(=O)N1C[C@H](O)C[C@H]1C(=O)NCc1ccc(-c2c(C)n[nH]c2C)cc1)C(C)(C)C]

In some embodiments, LBM is

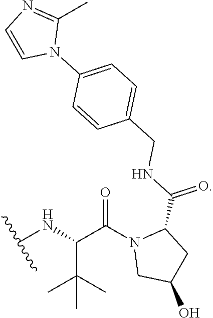 [SMILES: *N[C@H](C(=O)N1C[C@H](O)C[C@H]1C(=O)NCc1ccc(-n2ccnc2C)cc1)C(C)(C)C]

In some embodiments, LBM is

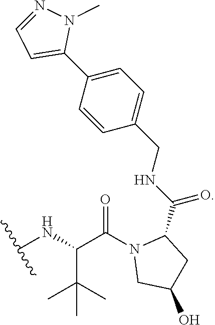 [SMILES: *N[C@H](C(=O)N1C[C@H](O)C[C@H]1C(=O)NCc1ccc(-c2ccnn2C)cc1)C(C)(C)C]

In some embodiments, LBM is

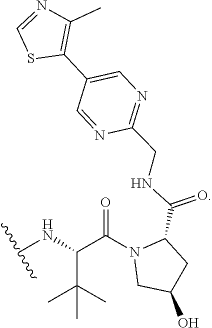 [SMILES: *N[C@H](C(=O)N1C[C@H](O)C[C@H]1C(=O)NCc1ncc(-c2scnc2C)cn1)C(C)(C)C]

In some embodiments, LBM is

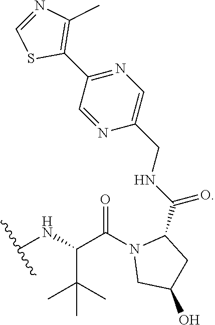 [SMILES: *N[C@H](C(=O)N1C[C@H](O)C[C@H]1C(=O)NCc1cnc(-c2scnc2C)cn1)C(C)(C)C]

In some embodiments, LBM is

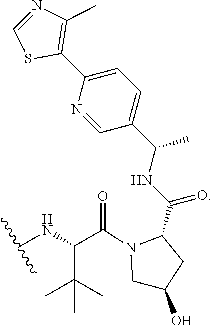 [SMILES: *N[C@H](C(=O)N1C[C@H](O)C[C@H]1C(=O)N[C@@H](C)c1ccc(-c2scnc2C)nc1)C(C)(C)C]

In some embodiments, LBM is

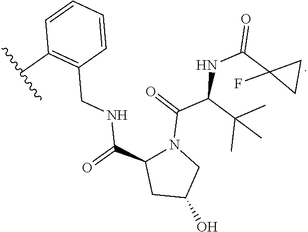 [SMILES: *c1ccccc1CNC(=O)[C@@H]1C[C@@H](O)CN1C(=O)[C@@H](NC(=O)C1(F)CC1)C(C)(C)C]

In some embodiments, LBM is

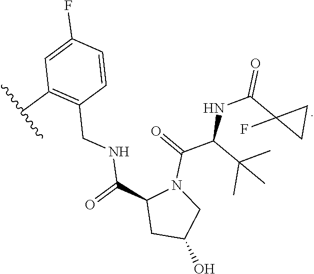 [SMILES: *c1cc(F)ccc1CNC(=O)[C@@H]1C[C@@H](O)CN1C(=O)[C@@H](NC(=O)C1(F)CC1)C(C)(C)C]

In some embodiments, LBM is

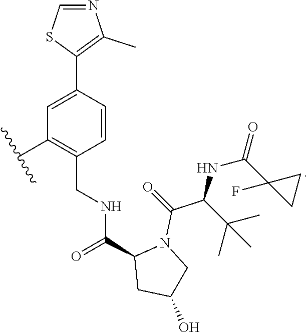 [SMILES: *c1cc(-c2scnc2C)ccc1CNC(=O)[C@@H]1C[C@@H](O)CN1C(=O)[C@@H](NC(=O)C1(F)CC1)C(C)(C)C]

In some embodiments, LBM is

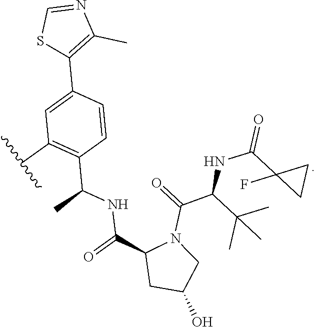 [SMILES: *c1cc(-c2scnc2C)ccc1[C@H](C)NC(=O)[C@@H]1C[C@@H](O)CN1C(=O)[C@@H](NC(=O)C1(F)CC1)C(C)(C)C]

In some embodiments, LBM is

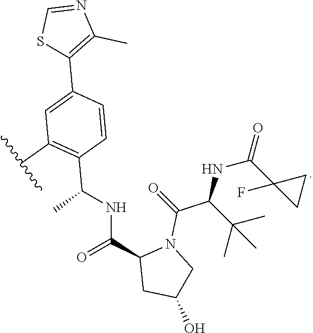 [SMILES: *c1cc(-c2scnc2C)ccc1[C@@H](C)NC(=O)[C@@H]1C[C@@H](O)CN1C(=O)[C@@H](NC(=O)C1(F)CC1)C(C)(C)C]

In some embodiments, LBM is

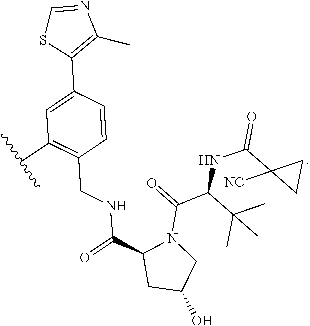 [SMILES: *c1cc(-c2scnc2C)ccc1CNC(=O)[C@@H]1C[C@@H](O)CN1C(=O)[C@@H](NC(=O)C1(C#N)CC1)C(C)(C)C]

In some embodiments, LBM is

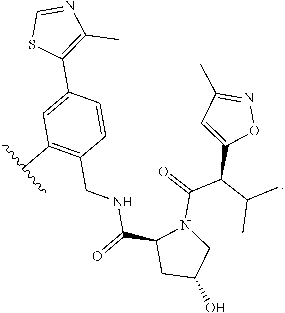 [SMILES: *c1cc(-c2scnc2C)ccc1CNC(=O)[C@@H]1C[C@@H](O)CN1C(=O)[C@@H](c1cc(C)no1)C(C)C]

In some embodiments, LBM is

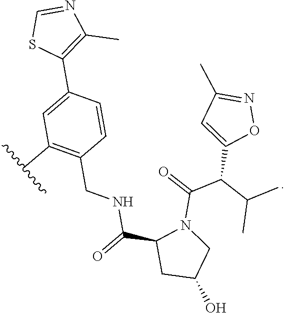 [SMILES: *c1cc(-c2scnc2C)ccc1CNC(=O)[C@@H]1C[C@@H](O)CN1C(=O)[C@H](c1cc(C)no1)C(C)C]

In some embodiments, LBM is

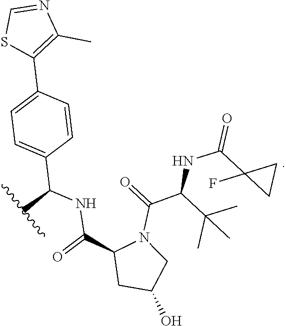 [SMILES: *[C@H](NC(=O)[C@@H]1C[C@@H](O)CN1C(=O)[C@@H](NC(=O)C1(F)CC1)C(C)(C)C)c1ccc(-c2scnc2C)cc1]

In some embodiments, LBM is

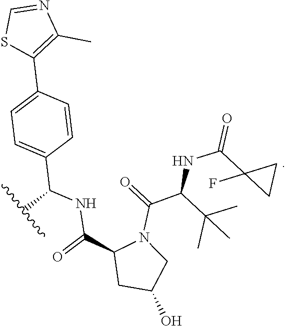 [SMILES: *[C@@H](NC(=O)[C@@H]1C[C@@H](O)CN1C(=O)[C@@H](NC(=O)C1(F)CC1)C(C)(C)C)c1ccc(-c2scnc2C)cc1]

In some embodiments, LBM is

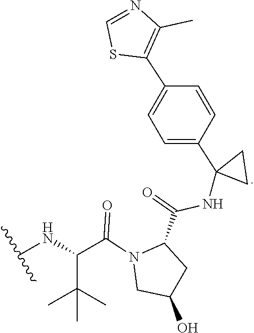 [SMILES: *N[C@H](C(=O)N1C[C@H](O)C[C@H]1C(=O)NC1(c2ccc(-c3scnc3C)cc2)CC1)C(C)(C)C]

In some embodiments, LBM is

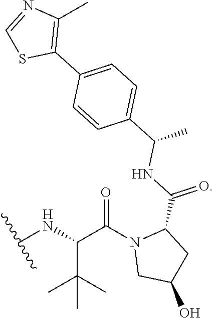 [SMILES: *N[C@H](C(=O)N1C[C@H](O)C[C@H]1C(=O)N[C@@H](C)c1ccc(-c2scnc2C)cc1)C(C)(C)C]

In some embodiments, LBM is

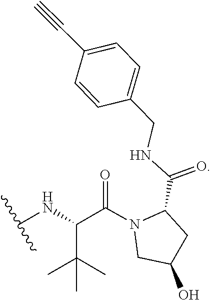 [SMILES: *N[C@H](C(=O)N1C[C@H](O)C[C@H]1C(=O)NCc1ccc(C#C)cc1)C(C)(C)C]

In some embodiments, LBM is

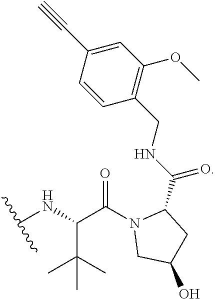 [SMILES: *N[C@H](C(=O)N1C[C@H](O)C[C@H]1C(=O)NCc1ccc(C#C)cc1OC)C(C)(C)C]

In some embodiments, LBM is

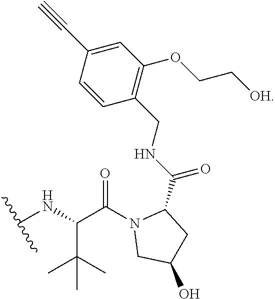 [SMILES: *N[C@H](C(=O)N1C[C@H](O)C[C@H]1C(=O)NCc1ccc(C#C)cc1OCCO)C(C)(C)C]

In some embodiments, LBM is

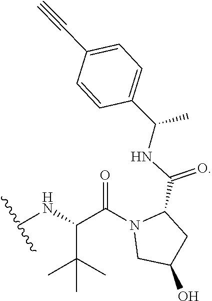 [SMILES: *N[C@H](C(=O)N1C[C@H](O)C[C@H]1C(=O)N[C@@H](C)c1ccc(C#C)cc1)C(C)(C)C]

In some embodiments, LBM is

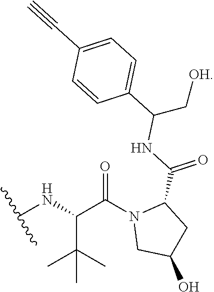 [SMILES: *N[C@H](C(=O)N1C[C@H](O)C[C@H]1C(=O)NC(CO)c1ccc(C#C)cc1)C(C)(C)C]

In some embodiments, LBM is

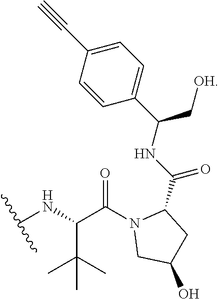 [SMILES: *N[C@H](C(=O)N1C[C@H](O)C[C@H]1C(=O)N[C@H](CO)c1ccc(C#C)cc1)C(C)(C)C]

In some embodiments, LBM is

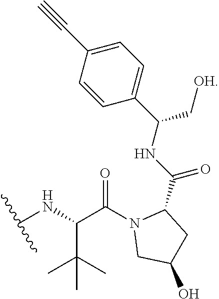 [SMILES: *N[C@H](C(=O)N1C[C@H](O)C[C@H]1C(=O)N[C@@H](CO)c1ccc(C#C)cc1)C(C)(C)C]

In some embodiments, LBM is

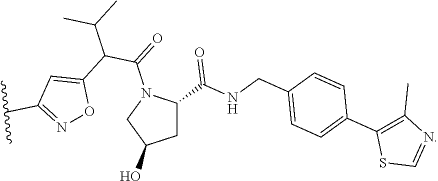 [SMILES: *c1cc(C(C(=O)N2C[C@H](O)C[C@H]2C(=O)NCc2ccc(-c3scnc3C)cc2)C(C)C)on1]

In some embodiments, LBM is

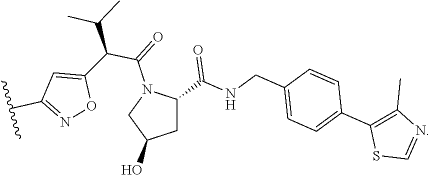 [SMILES: *c1cc([C@H](C(=O)N2C[C@H](O)C[C@H]2C(=O)NCc2ccc(-c3scnc3C)cc2)C(C)C)on1]

In some embodiments, LBM is

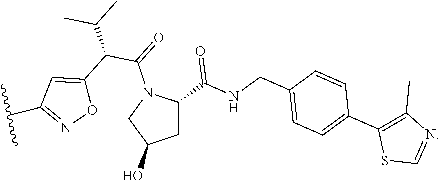 [SMILES: *c1cc([C@@H](C(=O)N2C[C@H](O)C[C@H]2C(=O)NCc2ccc(-c3scnc3C)cc2)C(C)C)on1]

In some embodiments, LBM is

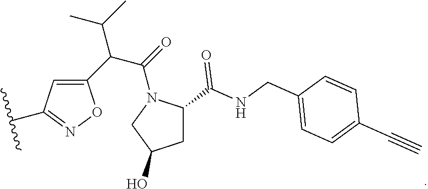 [SMILES: *c1cc(C(C(=O)N2C[C@H](O)C[C@H]2C(=O)NCc2ccc(C#C)cc2)C(C)C)on1]

In some embodiments, LBM is

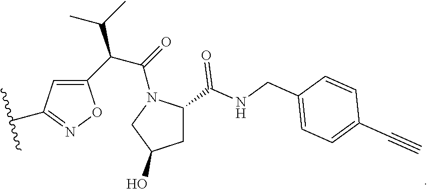 [SMILES: *c1cc([C@H](C(=O)N2C[C@H](O)C[C@H]2C(=O)NCc2ccc(C#C)cc2)C(C)C)on1]

In some embodiments, LBM is

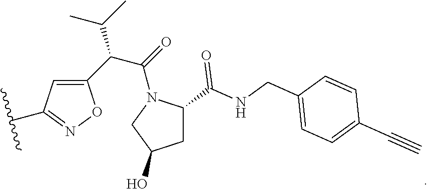 [SMILES: *c1cc([C@@H](C(=O)N2C[C@H](O)C[C@H]2C(=O)NCc2ccc(C#C)cc2)C(C)C)on1]

In some embodiments, LBM is

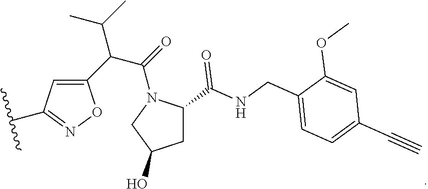 [SMILES: *c1cc(C(C(=O)N2C[C@H](O)C[C@H]2C(=O)NCc2ccc(C#C)cc2OC)C(C)C)on1]

In some embodiments, LBM is

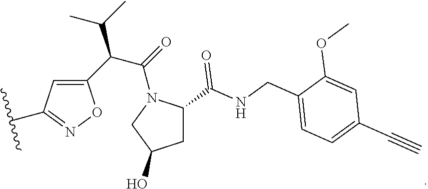 [SMILES: *c1cc([C@H](C(=O)N2C[C@H](O)C[C@H]2C(=O)NCc2ccc(C#C)cc2OC)C(C)C)on1]

In some embodiments, LBM is

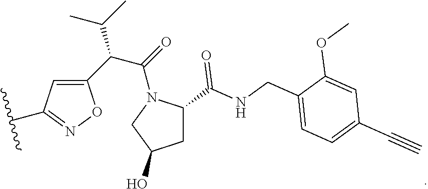 [SMILES: *c1cc([C@@H](C(=O)N2C[C@H](O)C[C@H]2C(=O)NCc2ccc(C#C)cc2OC)C(C)C)on1]

In some embodiments, LBM is

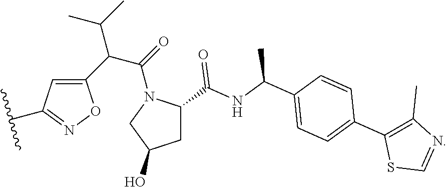 [SMILES: *c1cc(C(C(=O)N2C[C@H](O)C[C@H]2C(=O)N[C@@H](C)c2ccc(-c3scnc3C)cc2)C(C)C)on1]

In some embodiments, LBM is

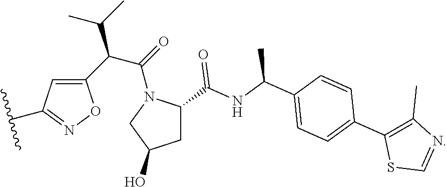 [SMILES: *c1cc([C@H](C(=O)N2C[C@H](O)C[C@H]2C(=O)N[C@@H](C)c2ccc(-c3scnc3C)cc2)C(C)C)on1]

In some embodiments, LBM is

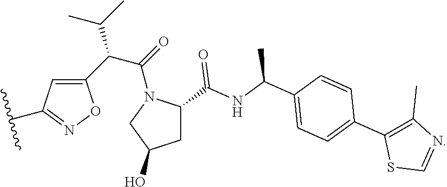 [SMILES: *c1cc([C@@H](C(=O)N2C[C@H](O)C[C@H]2C(=O)N[C@@H](C)c2ccc(-c3scnc3C)cc2)C(C)C)on1]

In some embodiments, LBM is

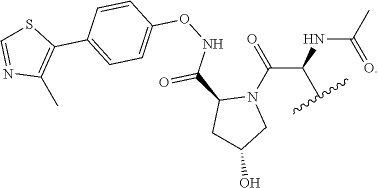 [SMILES: *[C@H](NC(C)=O)C(=O)N1C[C@H](O)C[C@H]1C(=O)NOc1ccc(-c2scnc2C)cc1]

In some embodiments, LBM is

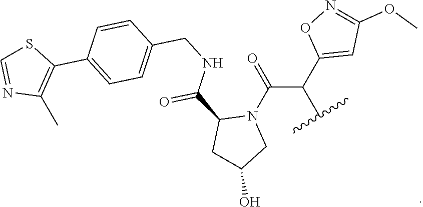 [SMILES: *C(C(=O)N1C[C@H](O)C[C@H]1C(=O)NCc1ccc(-c2scnc2C)cc1)c1cc(OC)no1]

In some embodiments, LBM is

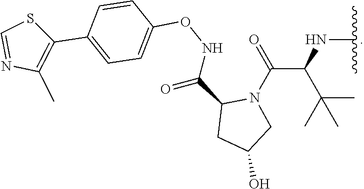 [SMILES: *N[C@H](C(=O)N1C[C@H](O)C[C@H]1C(=O)NOc1ccc(-c2scnc2C)cc1)C(C)(C)C]

In some embodiments, LBM is

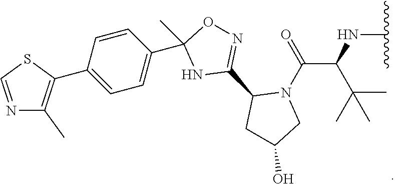 [SMILES: *N[C@H](C(=O)N1C[C@H](O)C[C@H]1C1=NOC(C)(c2ccc(-c3scnc3C)cc2)N1)C(C)(C)C]

In some embodiments, LBM is

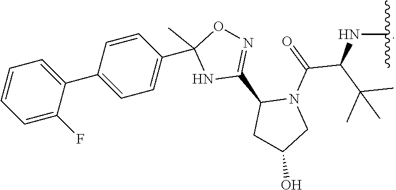 [SMILES: *N[C@H](C(=O)N1C[C@H](O)C[C@H]1C1=NOC(C)(c2ccc(-c3ccccc3F)cc2)N1)C(C)(C)C]

In some embodiments, LBM is

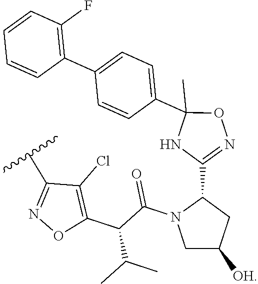 [SMILES: *c1noc([C@H](C(=O)N2C[C@H](O)C[C@H]2C2=NOC(C)(c3ccc(-c4ccccc4F)cc3)N2)C(C)C)c1Cl]

In some embodiments, LBM is

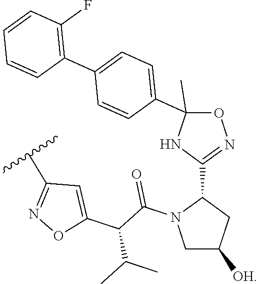 [SMILES: *c1cc([C@H](C(=O)N2C[C@H](O)C[C@H]2C2=NOC(C)(c3ccc(-c4ccccc4F)cc3)N2)C(C)C)on1]

In some embodiments, LBM is

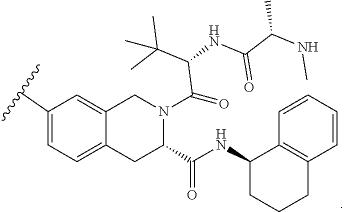 [SMILES: *c1ccc2c(c1)CN(C(=O)[C@@H](NC(=O)[C@H](C)NC)C(C)(C)C)[C@H](C(=O)N[C@@H]1CCCc3ccccc31)C2]

In some embodiments, LBM is

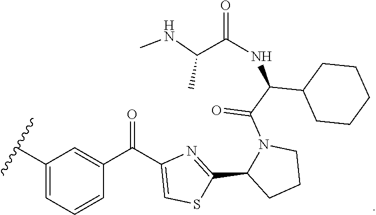 [SMILES: *c1cccc(C(=O)c2csc([C@@H]3CCCN3C(=O)[C@@H](NC(=O)[C@H](C)NC)C3CCCCC3)n2)c1]

In some embodiments, LBM is

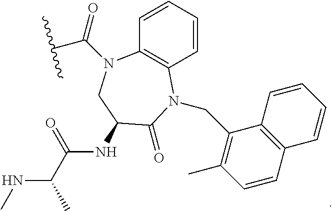 [SMILES: *C(=O)N1C[C@H](NC(=O)[C@H](C)NC)C(=O)N(Cc2c(C)ccc3ccccc23)c2ccccc21]

In some embodiments, LBM is

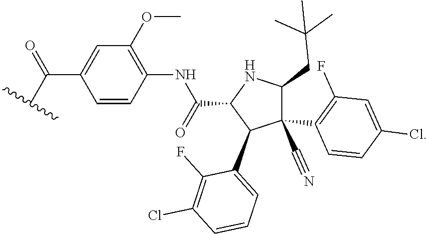 [SMILES: *C(=O)c1ccc(NC(=O)[C@@H]2N[C@@H](CC(C)(C)C)[C@](C#N)(c3ccc(Cl)cc3F)[C@H]2c2cccc(Cl)c2F)c(OC)c1]

In some embodiments, LBM is

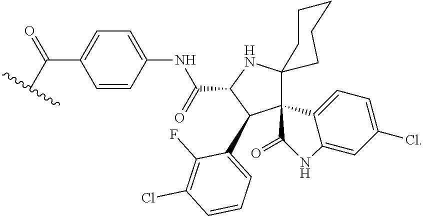 [SMILES: *C(=O)c1ccc(NC(=O)[C@@H]2NC3(CCCCC3)[C@@]3(C(=O)Nc4cc(Cl)ccc43)[C@H]2c2cccc(Cl)c2F)cc1]

In some embodiments, LBM is

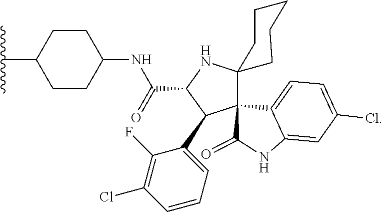 [SMILES: *C1CCC(NC(=O)[C@@H]2NC3(CCCCC3)[C@@]3(C(=O)Nc4cc(Cl)ccc43)[C@H]2c2cccc(Cl)c2F)CC1]

In some embodiments, LBM is

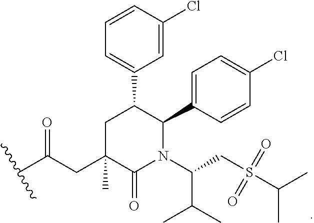 [SMILES: *C(=O)C[C@@]1(C)C[C@H](c2cccc(Cl)c2)[C@@H](c2ccc(Cl)cc2)N([C@H](CS(=O)(=O)C(C)C)C(C)C)C1=O]

In some embodiments, LBM is

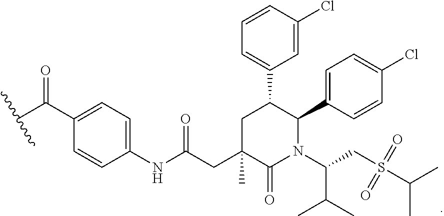 [SMILES: *C(=O)c1ccc(NC(=O)C[C@@]2(C)C[C@H](c3cccc(Cl)c3)[C@@H](c3ccc(Cl)cc3)N([C@H](CS(=O)(=O)C(C)C)C(C)C)C2=O)cc1]

In some embodiments, LBM is

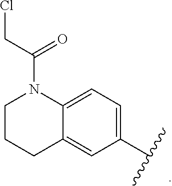 [SMILES: *c1ccc2c(c1)CCCN2C(=O)CCl]

In some embodiments, LBM is

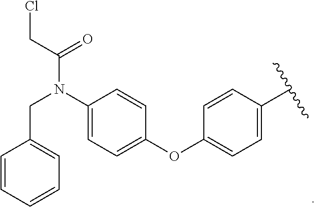 [SMILES: *c1ccc(Oc2ccc(N(Cc3ccccc3)C(=O)CCl)cc2)cc1]

In some embodiments, LBM is

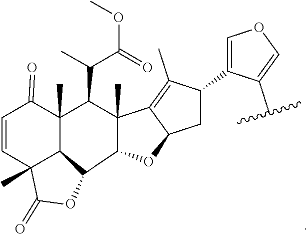 [SMILES: *c1cocc1[C@@H]1C[C@H]2O[C@@H]3[C@@H]4OC(=O)[C@]5(C)C=CC(=O)[C@](C)([C@@H](C(C)C(=O)OC)[C@]3(C)C2=C1C)[C@@H]45]

In some embodiments, LBM is

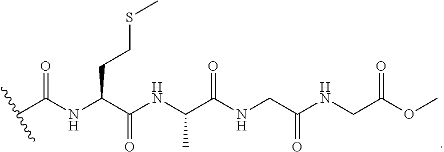 [SMILES: *C(=O)N[C@@H](CCSC)C(=O)N[C@@H](C)C(=O)NCC(=O)NCC(=O)OC]

In some embodiments, LBM is

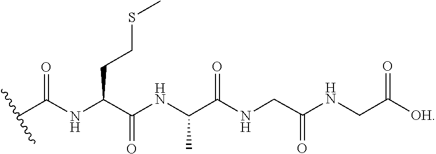 [SMILES: *C(=O)N[C@@H](CCSC)C(=O)N[C@@H](C)C(=O)NCC(=O)NCC(=O)O]

In some embodiments, LBM is

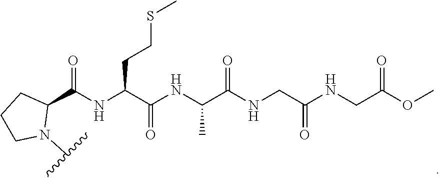 [SMILES: *N1CCC[C@H]1C(=O)N[C@@H](CCSC)C(=O)N[C@@H](C)C(=O)NCC(=O)NCC(=O)OC]

In some embodiments, LBM is

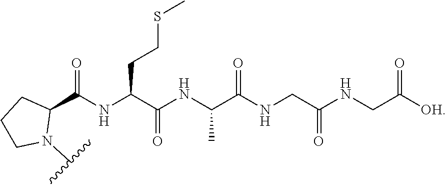 [SMILES: *N1CCC[C@H]1C(=O)N[C@@H](CCSC)C(=O)N[C@@H](C)C(=O)NCC(=O)NCC(=O)O]

In some embodiments, LBM is

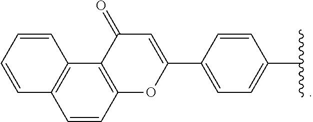 [SMILES: *c1ccc(-c2cc(=O)c3c(ccc4ccccc43)o2)cc1]

In some embodiments, LBM is

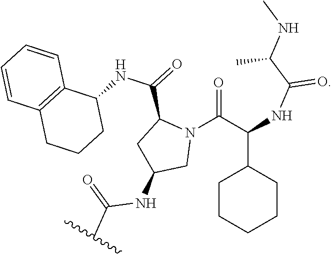 [SMILES: *C(=O)N[C@H]1C[C@@H](C(=O)N[C@@H]2CCCc3ccccc32)N(C(=O)[C@@H](NC(=O)[C@H](C)NC)C2CCCCC2)C1]

In some embodiments, LBM is

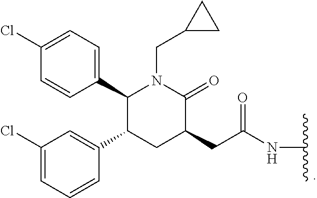 [SMILES: *NC(=O)C[C@H]1C[C@H](c2cccc(Cl)c2)[C@@H](c2ccc(Cl)cc2)N(CC2CC2)C1=O]

In some embodiments, LBM is

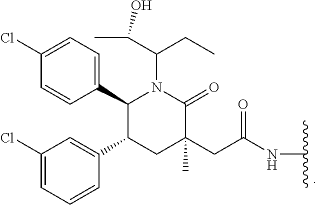 [SMILES: *NC(=O)C[C@@]1(C)C[C@H](c2cccc(Cl)c2)[C@@H](c2ccc(Cl)cc2)N(C(CC)[C@H](C)O)C1=O]

In some embodiments, LBM is

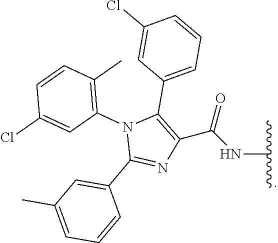 [SMILES: *NC(=O)c1nc(-c2cccc(C)c2)n(-c2cc(Cl)ccc2C)c1-c1cccc(Cl)c1]

In some embodiments, LBM is

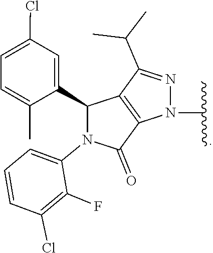 [SMILES: *n1nc(C(C)C)c2c1C(=O)N(c1cccc(Cl)c1F)[C@H]2c1cc(Cl)ccc1C]

In some embodiments, LBM is

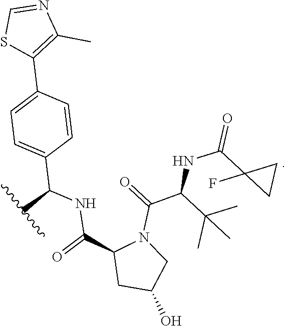 [SMILES: *[C@H](NC(=O)[C@@H]1C[C@@H](O)CN1C(=O)[C@@H](NC(=O)C1(F)CC1)C(C)(C)C)c1ccc(-c2scnc2C)cc1]

In certain embodiments, the present invention provides a compound of formula I, wherein LBM is a CRBN E3 ubiquitin ligase binding moiety thereby forming a compound of formula I-qqq:

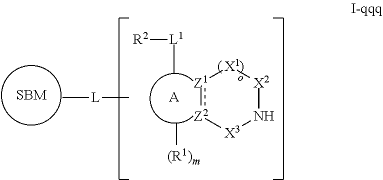

or a pharmaceutically acceptable salt thereof, wherein L and SBM are as defined above and described in embodiments herein, wherein:
each X¹ is independently —CH₂—, —O—, —NR—, —CF₂—,

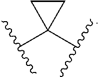 [SMILES: *C1(*)CC1]

### —C(O)—, —C(S)—, or

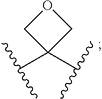 [SMILES: *C1(*)COC1]

X² and X³ are independently —CH₂—, —C(O)—, —C(S)— or

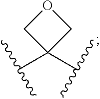 [SMILES: *C1(*)COC1]

Z¹ and Z² are independently a carbon atom or a nitrogen atom;
   Ring A is a fused ring selected from benzo, a 4-6 membered saturated or partially unsaturated carbocyclic or heterocyclic ring having 1-3 heteroatoms independently selected from nitrogen, oxygen, and sulfur, and a 5-6 membered heteroaryl ring having 1-4 heteroatoms independently selected from nitrogen, oxygen, and sulfur;
L¹ is a covalent bond or a C₁₋₃ bivalent straight or branched saturated or unsaturated hydrocarbon chain wherein 1-2 methylene units of the chain are independently and optionally replaced with —O—, —S—, —C(O)—, —C(S)—, —CR₂—, —CRF—, —CF₂—, —NR—, or —S(O)₂—;
each R¹ is independently selected from hydrogen, deuterium. R⁴, halogen, —CN, —NO₂, —OR, —SR, —NR₂, —S(O)₂R, —S(O)₂NR₂, —S(O)R, —CF₂R, —CRY, —CF₃, —CR₂(OR), —CR₂(NR₂), —C(O)R, —C(O)OR, —C(O)NR₂, —C(O)N(R)OR, —OC(O)R, —OC(O)NR₂, —C(S)NR₂, —N(R)C(O)OR, —N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, —Si(OR)R₂, and —SiR₃; or
   two R¹ groups are optionally taken together to form an optionally substituted 5-8 membered partially unsaturated or aryl fused ring having 0-2 heteroatoms independently selected from nitrogen, oxygen, or sulfur;
each R is independently selected from hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same carbon or nitrogen are optionally taken together with their intervening atoms to form an optionally substituted 4-7 membered saturated, partially unsaturated, or heteroaryl ring having 0-3 heteroatoms, in addition to the carbon or nitrogen, independently selected from nitrogen, oxygen, and sulfur;
R² is selected from

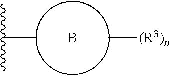

or hydrogen:
Ring B is phenyl, a 4-10 membered saturated or partially unsaturated mono- or bicyclic carbocyclic or heterocyclic ring having 1-3 heteroatoms independently selected from nitrogen, oxygen, and sulfur, or a 5-6 membered heteroaryl ring having 1-4 heteroatoms independently selected from nitrogen, oxygen, and sulfur, wherein Ring B is further optionally substituted with 1-2 oxo groups;
each R³ is independently selected from hydrogen, deuterium. R⁴, halogen, —CN, —NO₂, —OR. —SR, —NR₂, —S(O)₂R, —S(O)₂NR₂, —S(O)R, —CF₂R, —CF₃, —CR₂(OR), —CR₂(NR₂), —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, and —SiR₃:
each R⁴ is independently selected from an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
[symbol] is a single or double bond;
m is 0, 1, 2, 3 or 4;
n is 0, 1, 2, 3 or 4; and
is 0, 1, or 2.

As defined above and described herein each X¹ is independently a covalent bond, —CH₂—, —O—, —NR—, —CF₂—,

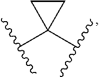 [SMILES: *C1(*)CC1]

### —C(O)—, —C(S)—, or

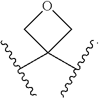 [SMILES: *C1(*)COC1]

In some embodiments, X¹ is a covalent bond. In some embodiments, X¹ is —CH₂—. In some embodiments, X¹ is —O—. In some embodiments, X¹ is —NR—. In some embodiments, X is —CF₂—. In some embodiments, X¹ is

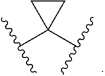 [SMILES: *C1(*)CC1]

In some embodiments, X is —C(O)—. In some embodiments, X¹ is —C(S)—. In some embodiments, X¹ is

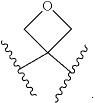 [SMILES: *C1(*)COC1]

In certain embodiments, X¹ is selected from those shown in the compounds of Table 1.

As defined above and described herein, X² and X³ are independently —CH₂—, —C(O)—, —C(S)—, or

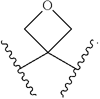 [SMILES: *C1(*)COC1]

In some embodiments, X² and X³ are independently —CH₂—. In some embodiments, X² and X³ are independently —C(O)—. In some embodiments, X² and X³ are independently —C(S)—. In some embodiments, X² and X³ are independently

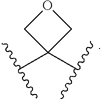 [SMILES: *C1(*)COC1]

In certain embodiments, X and X³ are independently selected from those shown in the compounds of Table 1.

As defined above and described herein, X⁴ is a covalent bond, —CH₂—, —CR₂—, —O—, —NR—, —CF₂—,

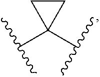 [SMILES: *C1(*)CC1]

### —C(O)—, —C(S)—, or

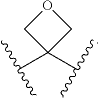 [SMILES: *C1(*)COC1]

As define above and described herein. Z¹ and Z² are independently a carbon atom or a nitrogen atom.

In some embodiments, Z¹ and Z² are independently a carbon atom. In some embodiments, Z¹ and Z² are independently a carbon atom.

In certain embodiments, Z¹ and Z² are independently selected from those shown in the compounds of Table 1.

As defined above and described herein, Ring A is fused ring selected from benzo or a 5-6 membered heteroaryl ring having 1-4 heteroatoms independently selected from nitrogen, oxygen, and sulfur.

In some embodiments, Ring A is benzo. In some embodiments, Ring A is a 5-6 membered heteroaryl ring having 1-4 heteroatoms independently selected from nitrogen, oxygen, and sulfur.

In certain embodiments, Ring A is selected from those shown in the compounds of Table 1.

As defined above and described herein, L¹ is a covalent bond or a C₁₋₃ bivalent straight or branched saturated or unsaturated hydrocarbon chain wherein 1-2 methylene units of the chain are independently and optionally replaced with —O—, —S—, —C(O)—, —C(S)—, —CR₂—, —CRF—, —CF₂—, —NR—, or —S(O)₂—.

In some embodiments, L¹ is a covalent bond. In some embodiments, L¹ is a C₁₋₃ bivalent straight or branched saturated or unsaturated hydrocarbon chain wherein 1-2 methylene units of the chain arc independently and optionally replaced with —O—, —S—, —C(O)—, —C(S)—, —CR₂—, —CRF—, —CF₂—, —NR—, or —S(O)₂—.

In some embodiments, L¹ is —C(O)—.

In certain embodiments, L¹ is selected from those shown in the compounds of Table 1.

As defined above and described herein, each R¹ is independently selected from hydrogen, deuterium, R⁴, halogen, —CN, —NO₂, —OR, —SR, —NR₂, —S(O)₂R, —S(O)₂NR₂, —S(O)R, —CF₂R, —CF₃, —CR₂(OR), —CR₂(NR₂), —C(O)R, —C(O)OR, —C(O)NR₂, —C(O)N(R)OR, —OC(O)R, —OC(O)NR₂, —C(S)NR₂, —N(R)C(O)OR, —N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, —Si(OR)R₂, and —SiR₃, or two R¹ groups are optionally taken together to form an optionally substituted 5-8 membered partially unsaturated or aryl fused ring having 0-2 heteroatoms independently selected from nitrogen, oxygen, or sulfur.

In some embodiments, R¹ is hydrogen. In some embodiments. R¹ is deuterium. In some embodiments, R¹ is R⁴. In some embodiments, R¹ is halogen. In some embodiments, R¹ is —CN. In some embodiments, R¹ is —NO₂. In some embodiments, R¹ is —OR. In some embodiments, R¹ is —SR. In some embodiments, R¹ is —NR₂. In some embodiments, R¹ is —S(O)₂R. In some embodiments, R¹ is —S(O)₂NR₂. In some embodiments, R¹ is —S(O)R. In some embodiments, R¹ is —CF₂R. In some embodiments, R¹ is —CF₃. In some embodiments, R¹ is —CR₂(OR). In some embodiments, R¹ is —CR₂(NR₂). In some embodiments, R¹ is —C(O)R. In some embodiments, R¹ is —C(O)OR. In some embodiments, R¹ is —C(O)NR₂. In some embodiments, R¹ is —C(O)N(R)OR. In some embodiments, R¹ is —OC(O)R. In some embodiments, R¹ is —OC(O)NR₂. In some embodiments, R¹ is —C(S)NR₂. In some embodiments, R¹ is —N(R)C(O)OR. In some embodiments, R¹ is —N(R)C(O)R. In some embodiments, R¹ is —N(R)C(O)NR₂. In some embodiments. R¹ is —N(R)S(O)₂R. In some embodiments, R¹ is —OP(O)R₂. In some embodiments, R¹ is —OP(O)(OR)₂. In some embodiments, R¹ is —OP(O)(OR)NR₂. In some embodiments, R¹ is —OP(O)(NR₂)₂. In some embodiments, R¹ is —Si(OR)R₂. In some embodiments, R¹ is —SiR₃. In some embodiments, two R¹ groups are optionally taken together to form an optionally substituted 5-8 membered partially unsaturated or aryl fused ring having 0-2 heteroatoms independently selected from nitrogen, oxygen, or sulfur.

In certain embodiments, each R¹ is independently selected from those shown in the compounds of Table 1.

As defined above and described here, each R is independently selected from hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or two R groups on the same carbon or nitrogen are optionally taken together with their intervening atoms to form an optionally substituted 4-7 membered saturated, partially unsaturated, or heteroaryl ring having 0-3 heteroatoms, in addition to the carbon or nitrogen, independently selected from nitrogen, oxygen, and sulfur.

In some embodiments, R is hydrogen. In some embodiments, R is an optionally substituted C₁₋₆ aliphatic. In some embodiments, R is an optionally substituted phenyl. In some embodiments, R is an optionally substituted 4-7 membered saturated or partially unsaturated heterocyclic having 1-2 heteroatoms independently selected from nitrogen, oxygen, and sulfur. In some embodiments, R is an optionally substituted a 5-6 membered heteroaryl ring having 1-4 heteroatoms independently selected from nitrogen, oxygen, and sulfur. In some embodiments, two R groups on the same carbon or nitrogen are optionally taken together with their intervening atoms to form an optionally substituted 4-7 membered saturated, partially unsaturated, or heteroaryl ring having 0-3 heteroatoms, in addition to the carbon or nitrogen, independently selected from nitrogen, oxygen, and sulfur.

As defined above and described herein, R² is selected from

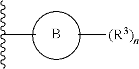

or hydrogen.

In some embodiment R² is

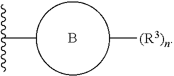

In some embodiments. R² is hydrogen.

In certain embodiments, R² is selected from those shown in the compounds of Table 1.

As defined above and described herein, Ring B is phenyl, a 4-10 membered saturated or partially unsaturated mono- or bicyclic carbocyclic or heterocyclic ring having 1-3 heteroatoms independently selected from nitrogen, oxygen, and sulfur, or a 5-6 membered heteroaryl ring having 1-4 heteroatoms independently selected from nitrogen, oxygen, and sulfur, wherein Ring B is further optionally substituted with 1-2 oxo groups.

In some embodiments, Ring B is phenyl. In some embodiments, Ring B is a 4-10 membered saturated or partially unsaturated mono- or bicyclic carbocyclic or heterocyclic ring having 1-3 heteroatoms independently selected from nitrogen, oxygen, and sulfur In some embodiments, Ring B is a 5-6 membered heteroaryl ring having 1-4 heteroatoms independently selected from nitrogen, oxygen, and sulfur. In some embodiments, Ring B is further optionally substituted with 1-2 oxo groups.

In certain embodiments, Ring B is selected from those shown in the compounds of Table 1.

As defined above and described herein, each R³ is independently selected from hydrogen, deuterium, R⁴ halogen, —CN, —NO₂, —OR, —SR, —NR₂, —S(O)₂R, —S(O)₂NR₂, —S(O)R, —CF₂R, —CF₃, —CR₂(OR), —CR₂(NR₂), —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, and —SiR₃.

In some embodiments, R³ is hydrogen. In some embodiments. R³ is deuterium. In some embodiments. R³ is R⁴. In some embodiments, R³ is halogen. In some embodiments. R³ is —CN. In some embodiments, R³ is —NO₂. In some embodiments, R³ is —OR. In some embodiments, R³ is —SR. In some embodiments, R³ is —NR₂. In some embodiments, R³ is —S(O)₂R. In some embodiments, R³ is —S(O)₂NR₂. In some embodiments, R³ is —S(O)R. In some embodiments, R³ is —CF₂R. In some embodiments, R³ is —CF₃. In some embodiments, R³ is —CR₂(OR). In some embodiments, R³ is —CR₂(NR₂). In some embodiments, R³ is —C(O)R. In some embodiments, R³ is —C(O)OR. In some embodiments, R³ is —C(O)NR₂. In some embodiments, R³ is —C(O)N(R)OR. In some embodiments, R³ is —OC(O)R. In some embodiments, R³ is —OC(O)NR₂. In some embodiments, R³ is —N(R)C(O)OR. In some embodiments, R³ is —N(R)C(O)R. In some embodiments, R³ is —N(R)C(O)NR₂. In some embodiments, R³ is —N(R)S(O)₂R. In some embodiments, R³ is —OP(O)R₂. In some embodiments, R³ is —OP(O)(OR)₂. In some embodiments, R³ is —OP(O)(OR)NR₂. In some embodiments, R³ is —OP(O)(NR₂)₂. In some embodiments, R³ is —SiR₃.

In certain embodiments. R³ is selected from those shown in the compounds of Table 1.

As defined above and described herein, each R⁴ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, R⁴ is an optionally substituted C₁₋₆ aliphatic. In some embodiments, R⁴ is an optionally substituted phenyl. In some embodiments, R⁴ is an optionally substituted 4-7 membered saturated or partially unsaturated heterocyclic ring having 1-2 heteroatoms independently selected from nitrogen, oxygen, and sulfur. In some embodiments, R⁴ is an optionally substituted 5-6 membered heteroaryl ring having 1-4 heteroatoms independently selected from nitrogen, oxygen, and sulfur.

In certain embodiments, R⁴ is selected from those shown in the compounds of Table 1.

As defined above and described herein, [symbol] is a single or double bond.

In some embodiments, [symbol] is a single bond. In some embodiments, [symbol] is a double bond.

In certain embodiments, [symbol] is selected from those shown in the compounds of Table 1.

As defined above and described herein, m is 0, 1, 2, 3 or 4.

In some embodiments, m is 0. In some embodiments, m is 1. In some embodiments, m is 2. In some embodiments, m is 3. In some embodiments, n is 4.

In certain embodiments, m is selected from those shown in the compounds of Table 1.

As defined above and described herein, n is 0, 1, 2, 3 or 4.

In some embodiments, n is 0. In some embodiments, n is 1. In some embodiments, n is 2. In some embodiments, n is 3. In some embodiments, n is 4.

In certain embodiments, n is selected from those shown in the compounds of Table 1.

As defined above and described herein, o is 0, 1, or 2.

In some embodiments, n is 0. In some embodiments, n is 1. In some embodiments, m is 2.

In certain embodiments, o is selected from those shown in the compounds of Table 1.

In some embodiments, the present invention provides a compound of formula I-qqq-A:

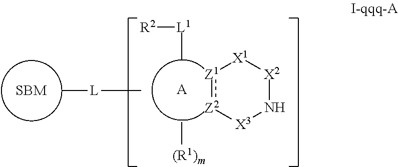

or a pharmaceutically acceptable salt thereof, wherein each of SBM, L, L¹, R¹, R², m, Z^{m}. Z², and X¹ is as defined above and described in embodiments herein, both singly and in combination.

In some embodiments, the present invention provides a compound of formula I-qqq-B:

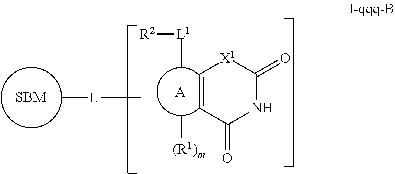

or a pharmaceutically acceptable salt thereof, wherein each of SBM, L, L¹, R¹, R², m, and X¹ is as defined above and described in embodiments herein, both singly and in combination.

In some embodiments, the present invention provides a compound of formula I-qqq, wherein Ring A is benzo, o is 1. X¹ is —CH₂—, X² and X³ are —C(O)—, and Z¹ and Z² are carbon atoms as shown, to provide a compound of formula I-qqq-1:

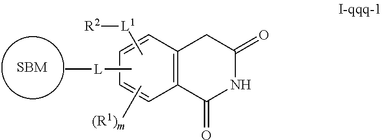

or a pharmaceutically acceptable salt thereof, wherein each of SBM, L, L¹, R¹, R², and m is as defined above and described in embodiments herein, both singly and in combination.

In some embodiments, the present invention provides a compound of formula I-qqq, wherein Ring A is benzo, o is 1, X¹, X² and X³ are —C(O)—, and Z¹ and Z² are carbon atoms as shown, to provide a compound of formula I-qqq-12:

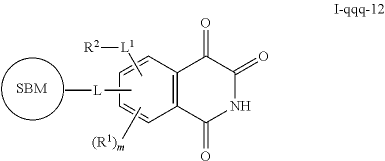

or a pharmaceutically acceptable salt thereof, wherein each of SBM, L, L¹, R¹, R², and m is as defined above and described in embodiments herein, both singly and in combination.

In some embodiments, LBM is

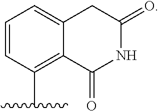 [SMILES: *c1cccc2c1C(=O)NC(=O)C2]

In some embodiments, LBM is

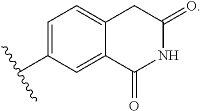 [SMILES: *c1ccc2c(c1)C(=O)NC(=O)C2]

In some embodiments, LBM is

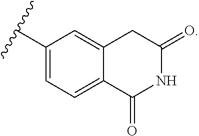 [SMILES: *c1ccc2c(c1)CC(=O)NC2=O]

In some embodiments, LBM is

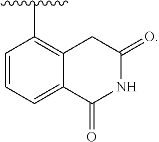 [SMILES: *c1cccc2c1CC(=O)NC2=O]

In some embodiments, LBM is

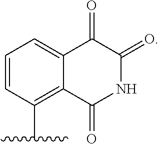 [SMILES: *c1cccc2c1C(=O)NC(=O)C2=O]

In some embodiments, LBM is

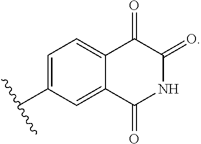 [SMILES: *c1ccc2c(c1)C(=O)NC(=O)C2=O]

In some embodiments, LBM is

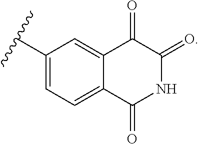 [SMILES: *c1ccc2c(c1)C(=O)C(=O)NC2=O]

In some embodiments, LBM is

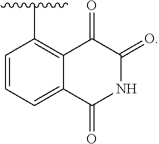 [SMILES: *c1cccc2c1C(=O)C(=O)NC2=O]

In some embodiments, LBM is selected from those in Table 1, below.

In certain embodiments, the present invention provides a compound of formula I, wherein LBM is a RPN13 binding moiety thereby forming a compound of formula I-rrr:

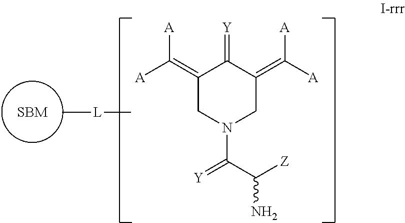

or a pharmaceutically acceptable salt thereof, wherein L and SBM are as defined above and described in embodiments herein, and wherein:
in each pair of A's, one A is hydrogen, and the other A is one of:
(i) phenyl, optionally substituted with 1-5 substituents selected from the group consisting of R¹, OR¹, NR¹R², S(O)_{q}R¹, SO₂R¹R², NR¹SO₂R², C(O)R¹, C(O)OR¹, C(O)NR¹R², NR¹C(O)R², NR¹C(O)OR², CF₃, and OCF₃;
(ii) naphthyl, optionally substituted with 1-5 substituents selected from the group consisting of R¹, OR¹, NR¹R², S(O)_{q}R¹, SO₂R¹R², NR¹SO₂R², C(O)R¹, C(O)OR¹, C(O)NR¹R₂, NR¹C(O)R², NR¹C(O)OR², CF₃, and OCF₃:
(iii) a 5 or 6 membered monocyclic heteroaryl group, having 1-3 heteroatoms selected from the group consisting of O, N, and S, optionally substituted with 1-3 substituents selected from the group consisting of R¹, OR¹, NR₁R², S(O)_{q}R¹, SO₂R¹R², NR₁SO₂R², C(O)R¹, C(O)OR, C(O)NR¹R², NR¹C(O)R², NR¹C(O)OR², CF₃, and OCF₃; and
(iv) an 8 to 10 membered bicyclic heteroalkyl group containing 1-3 heteroatoms selected from the group consisting of O, N, and S; and the second ring is fused to the first ring using 3 to 4 carbon atoms, and the bicyclic hetero aryl group is optionally substituted with 1-3 substituents selected from the group consisting of R¹, OR¹, NR¹R², SO₂R¹R², NR¹SO₂R², C(O)R¹, C(O)OR¹, C(O)NR¹R², NR¹C(O)R². NR¹C(O)OR², CF₃, and OCF₃;
wherein Y is selected from the group consisting of O, S, NR¹ and CR¹R²;
wherein R¹ and R² are selected from the group consisting of hydrogen, nitro, hydroxyl, carboxy, amino, halogen, cyano and C₁-C₁₄ linear or branched alkyl groups, that are optionally substituted with 1-3 substituents selected from the group consisting of C₁-C₁₄ linear or branched alkyl, up to perhalo substituted C₁-C₁₄ linear or branched alkyl, C₁-C₁₄ alkoxy, hydrogen, nitro, hydroxyl, carboxy, amino, C₁-C₁₄ alkylamino, C₁-C₁₄ dialkylamino, halogen, and cyano; and
wherein Z is selected from the group consisting of hydrogen; C₁-C₁₄ linear, branched, or cyclic alkyls; phenyl; benzyl, 1-5 substituted benzyl. C₁ to C₃ alkyl-phenyl, wherein the alkyl moiety is optionally substituted with halogen up to perhalo; up to perhalo substituted C₁ to C₁₋₄ linear or branched alkyls; —(CH₂)q-K, where K is a 5 or 6 membered monocyclic heterocyclic ring, containing 1 to 4 atoms selected from oxygen, nitrogen and sulfur, which is saturated, partially saturated, or aromatic, or an 8 to 10 membered bicyclic heteroaryl having 1-4 heteroatoms selected from the group consisting of O, N, and S, wherein said alkyl moiety is optionally substituted with halogen up to perhalo, and wherein the variable q is an integer ranging from 0 to 4,
as described and defined in WO 2019/165229, the entirety of each of which is herein incorporated by reference.

In certain embodiments, the present invention provides a compound of formula I, wherein LBM is a Ubr1 binding moiety as described in Shanmugasundaram, K. et al, J. Bio. Chem, 2019, doi: 10.1074/jbc.AC119.010790, the entirety of each of which is herein incorporated by reference, thereby forming a compound of formula I-sss-1 or I-sss-2:

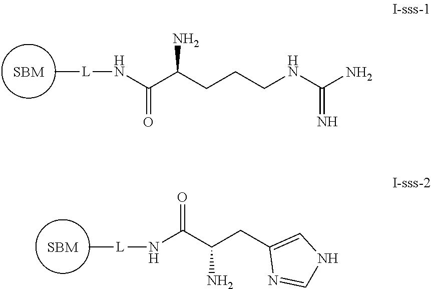

or a pharmaceutically acceptable salt thereof, wherein L and SBM are as defined above and described in embodiments herein.

In certain embodiments, the present invention provides a compound of formula I, wherein LBM is a CRBN E3 ubiquitin ligase binding moiety thereby forming a compound of formula I-uuu-1, I-uuu-2, I-uuu-3 or I-uuu-4:

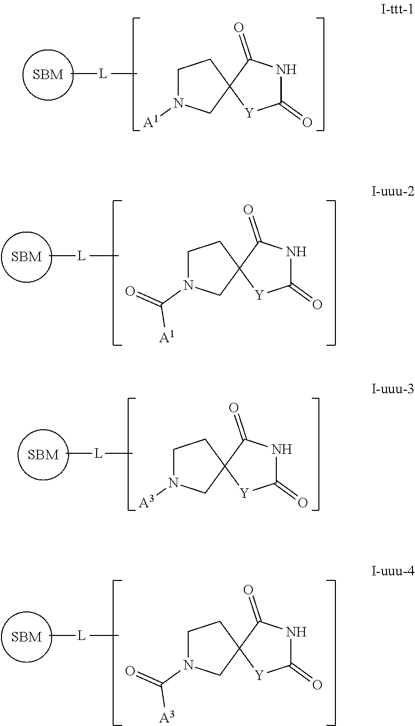

or a pharmaceutically acceptable salt thereof, wherein L and SBM are as defined above and described in embodiments herein, and wherein:
Y is NH or CH₂;
A¹ is selected from the group consisting of aryl and aryl substituted with R¹:
A³ is selected from the group consisting of heteroaryl and heteroaryl substituted with R²;
R¹ is selected from the group consisting of; —C(═O)—O—C₁₋₆-alkyl, —COOH, —NH—(C═O)—C₁₋₆-alkyl, —NH₂, and —NO₂:
R² is selected from the group consisting of; —COOH, —C(═O)—O—C₁₋₆-alkyl, —NH₂, and —NO₂,
as described and defined in WO 2019/236483, the entirety of each of which is herein incorporated by reference.

In certain embodiments, the present invention provides a compound of formula I, wherein LBM is human kelch-like ECH-associated protein 1 (KEAP1) thereby forming a compound of formula I-vvv:

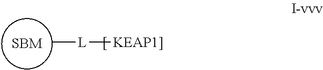

or a pharmaceutically acceptable salt thereof, wherein L and SBM are as defined above and described in embodiments herein, both singly and in combination.

In certain embodiments, the present invention provides a compound of formula I, wherein LBM is KEAP1 binding moiety as recited in Lu et al., Euro. J. Med. Chem., 2018, 146:251-9, thereby forming a compound of formula I-www:

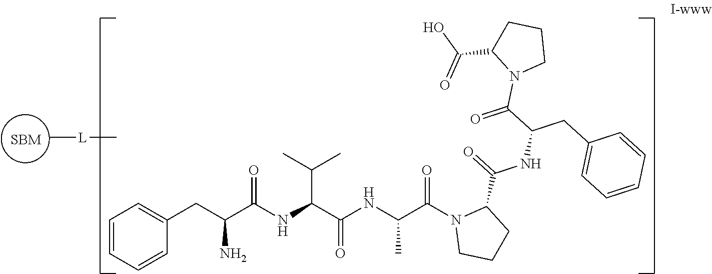

or a pharmaceutically acceptable salt thereof, wherein L and SBM are as defined above and described in embodiments herein, both singly and in combination.

In certain embodiments, the present invention provides a compound of formula I, wherein LBM is KEAP1-NRF2 binding moiety thereby forming a compound of formula I-xxx or I-xxx-2:

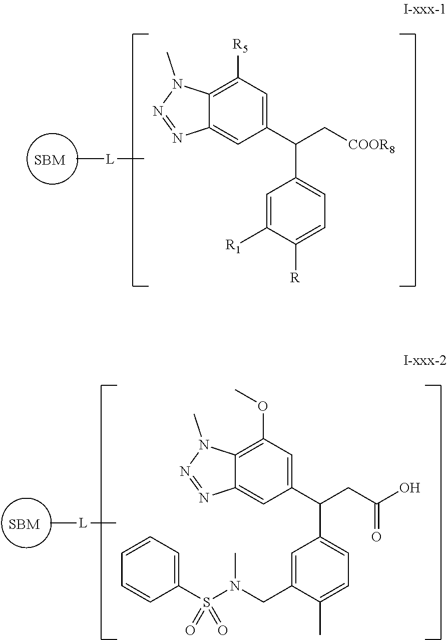

or a pharmaceutically acceptable salt thereof, wherein L and SBM are as defined above and described in embodiments herein, and wherein:
R is methyl or halo;
R is

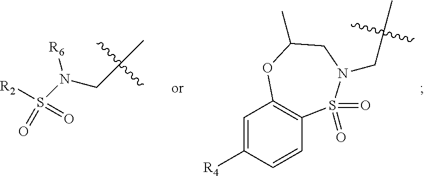

R² is methyl, or

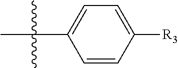

R³ is H;
R⁴ is H or halo;
R⁵ is methoxy or H;
R⁶ is H or methyl;
R⁸ is H, methyl or ethyl;
as described and defined in WO 2020/018788, the entirety of each of which is herein incorporated by reference.

In certain embodiments, the present invention provides a compound of formula I, wherein LBM is KEAP1-NRF2 binding moiety as recited in Tong et al., “Targeted Protein Degradation via a Covalent Reversible Degrader Based on Bardoxolone”, ChemRxiv 2020, thereby forming a compound of formula I-yyy-1 or I-yyy-2:

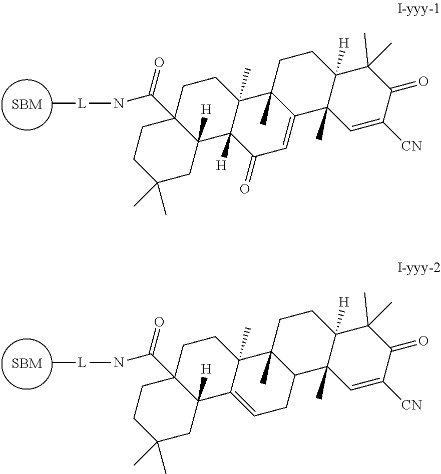

or a pharmaceutically acceptable salt thereof, wherein L and SBM are as defined above and described in embodiments herein, both singly and in combination.

In some embodiments, LBM is

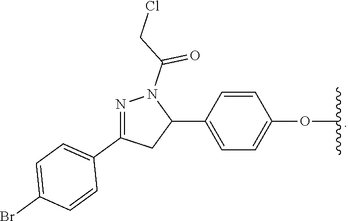 [SMILES: *Oc1ccc(C2CC(c3ccc(Br)cc3)=NN2C(=O)CCl)cc1]

In some embodiments, LBM is

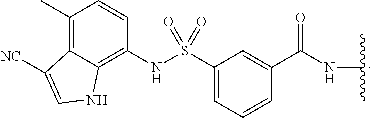 [SMILES: *NC(=O)c1cccc(S(=O)(=O)Nc2ccc(C)c3c(C#N)c[nH]c23)c1]

In some embodiments, the present disclosure provides a compound of formula I-zzz:

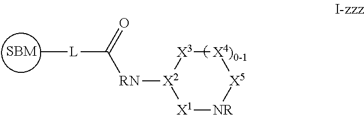

or a pharmaceutically acceptable salt thereof, wherein SBM, L, R, X¹, X², X³, X⁴, and X⁵ are as defined above and described herein.

In some embodiments, the present disclosure provides a compound of formula I-aaaa:

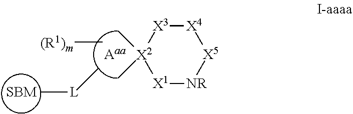

or a pharmaceutically acceptable salt thereof, wherein SBM, L, R, R¹, m, X¹, X², X³, X⁴, and X⁵ are as defined above and described herein; and:
Ring A^{aa} is a 3- to 10-membered saturated or partially unsaturated carbocyclyl or heterocyclyl ring having 1-4 heteroatoms independently selected from nitrogen, oxygen, or sulfur.

As defined above and described herein, Ring A^{aa} is a 3- to 10-membered saturated or partially unsaturated carbocyclyl or heterocyclyl ring having 1-4 heteroatoms independently selected from nitrogen, oxygen, or sulfur. In some embodiments, Ring A^{aa} is a 3- to 10-membered saturated or partially unsaturated carbocyclyl. In some embodiments, Ring A^{aa} is a 3- to 10-membered saturated or partially unsaturated heterocyclyl ring having 1-4 heteroatoms independently selected from nitrogen, oxygen, or sulfur. In some embodiments, Ring A^{aa} is a 5- to 6-membered saturated or partially unsaturated carbocyclyl. In some embodiments, Ring A^{aa} is a 5- to 6-membered saturated or partially unsaturated heterocyclyl ring having 1-4 heteroatoms independently selected from nitrogen, oxygen, or sulfur.

In some embodiments, Ring A^{aa} is a 5-membered saturated or partially unsaturated heterocyclyl ring having 1-4 heteroatoms independently selected from nitrogen, oxygen, or sulfur. In some embodiments, Ring A^{aa} is pyrrolidinyl. In some embodiments, Ring A^{aa} is 4,5-dihydrothiazolyl. In some embodiments, Ring A^{aa} is 4,5-dihydroisoxazolyl.

In some embodiments, a compound of formulae I-aaaa is of formula I-aaaa-1, I-aaaa-2, or I-aaaa-3:

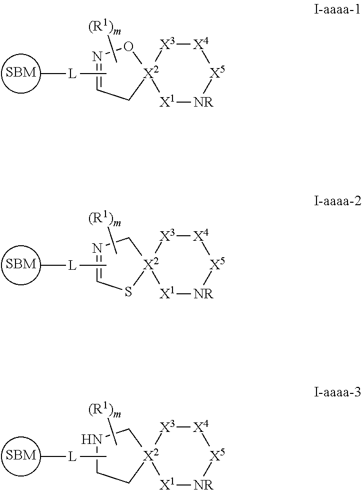

or a pharmaceutically acceptable salt thereof, wherein SBM, L, R, R¹, m, X¹, X², X³, X⁴, and X⁵ are as defined above and described herein.

In certain embodiments, the present invention provides a compound of formula I-b:

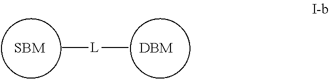

or a pharmaceutically acceptable salt thereof, wherein, SBM and L are as defined above and described herein; and
DBM is DCAF E3 ubiquitin ligase binding moiety capable of binding to DCAF1 protein.

As described above, in certain embodiments, the present invention provides a compound of formula I-b, wherein DBM is a compound of formula I-b-a or I-b-b:

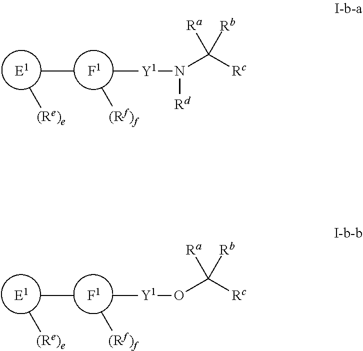

or a pharmaceutically acceptable salt thereof, wherein:
Ring E¹ is phenyl, naphthyl, a 4-9 membered partially unsaturated monocyclic, bicyclic, or bridged bicyclic carbocyclyl or heterocyclyl with 1-3 heteroatoms independently selected from nitrogen, oxygen, and sulfur, or a 5-9 membered monocyclic or bicyclic heteroarylenyl with 1-4 heteroatoms independently selected from nitrogen, oxygen and sulfur;
Ring F¹ is a 5-membered monocyclic heteroarylenyl with 1-3 heteroatoms independently selected from nitrogen, oxygen and sulfur.
Y¹ is a C₁₋₃ hydrocarbon chain wherein each methylene is optionally replaced with —CR₂—, —CR(OR)—, —C(O)—, —C(NR)—, —C(NOR)—, —S(O)—, or —S(O)₂—; or —C(OR)═ in formula I-b-a where R^{d} is absent:
R^{a} is hydrogen, an optionally substituted C₁₋₆ aliphatic, or

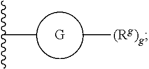

Ring G is phenyl, a 5-7 membered saturated or partially unsaturated carbocyclyl or heterocyclyl with 1-3 heteroatoms independently selected from nitrogen, oxygen, and sulfur, or a 5-6 membered heteroaryl with 1-4 heteroatoms independently selected from nitrogen, oxygen and sulfur;
R^{b} is hydrogen, an optionally substituted C₁₋₆ aliphatic, phenyl, or a 5-6 membered heteroaryl with 1-4 heteroatoms independently selected from nitrogen, oxygen and sulfur, or:
   R^{a} and R^{b} are taken together with their intervening atoms to form an optionally substituted 9-10 membered saturated or partially unsaturated bicyclic ring having 0-3 heteroatoms independently selected from nitrogen, oxygen, and sulfur, or:
   when Y¹ is —C(NR)—, R^{b} is taken together with R of —C(NR)— with their intervening atoms to form a 5-7 membered partially unsaturated heterocyclyl with 0-1 heteroatoms, in addition to the 2 heteroatoms within the heterocyclyl, independently selected from nitrogen, oxygen, and sulfur;
R^{c} is —CO₂R, —CONR₂, —CR₂CF₂R, —CR₂CONR₂, —CR₂C(O)R, —CR₂CO₂R, —CR₂NR₂, —CR₂OR, —CR₂SO₂NR₂, —CRS(O)R, —CR₂SO₂R, —CR₂S(O)(NR)R, —CR₂CN, —CR₂CR₂NR₂, —CR₂CR₂OR, —CR₂CR═NOR, —CR₂CR(OR)CR₂OR, or an optionally substituted group selected from phenyl; a 4-7 membered saturated or partially unsaturated carbocyclyl or heterocyclyl with 1-3 heteroatoms independently selected from nitrogen, oxygen, and sulfur: a 5-9 membered monocyclic or bicyclic heteroaryl with 1-4 heteroatoms independently selected from nitrogen, oxygen and sulfur; or:
   —(CR₂)₁₋₂—X^{a}, wherein X^{a} is halogen or an optionally substituted ring selected from phenyl: a 4-7 membered saturated or partially unsaturated carbocyclyl or heterocyclyl with 1-3 heteroatoms independently selected from nitrogen, oxygen, and sulfur; and a 5-9 membered monocyclic or bicyclic heteroaryl with 1-4 heteroatoms independently selected from nitrogen, oxygen and sulfur; or:
   R^{b} and R^{c} are taken together with their intervening atoms to form an optionally substituted 4-6 membered saturated or partially unsaturated carbocyclyl or heterocyclyl with 1-3 heteroatoms independently selected from nitrogen, oxygen, and sulfur; or:
   R^{a} is absent and R^{b} and R^{c} are taken together with their intervening atoms to form an optionally substituted phenyl; or:
   when Y¹ is —C(OR)=, R^{∘} is taken together with R of —C(OR)═ with their intervening atoms to form a 5-7 membered partially unsaturated heterocyclyl with 0-1 heteroatoms, in addition to the 2 heteroatoms within the heterocyclyl, independently selected from nitrogen, oxygen, and sulfur;
R^{d} is hydrogen or an optionally substituted C₁₋₆ aliphatic, or:
   when R¹ is —CR₂CONR₂, R^{d} is taken together with a single R of —CR₂CONR₂ with their intervening atoms to form a 5-7 membered saturated or partially unsaturated heterocyclyl with 0-3 heteroatoms, in addition to the nitrogen atom to which R^{d} is attached, independently selected from nitrogen, oxygen, and sulfur;
R^{e}, R^{f}, and R^{g} are each independently selected from hydrogen, oxo, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —C(NOR)R, —OC(O)R, —OC(O)NR₂, —OP(O)R₂, —OP(O)(OR)₂, —OP(O)(OR)NR₂, —OP(O)(NR₂)₂, —NRC(O)OR, —NRC(O)R, —NRC(O)N(R)₂, —NRS(O)₂R, —NP(O)R₂, —NRP(O)(OR)₂, —NRP(O)(OR)NR₂, —NRP(O)(NR₂)₂, —P(O)R₂, —P(O)(OR)₂, —P(O)(OR)NR₂, and —P(O)(NR₂)₂:
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur:
each R is independently hydrogen, or an optionally substituted group selected from C₁₋₆ aliphatic, phenyl, naphthyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are taken together with their intervening atoms to form an optionally substituted 3-7 membered saturated or partially unsaturated ring having 0-3 heteroatoms, in addition to the atom to which they are attached, independently selected from nitrogen, oxygen, and sulfur; and
each of e, f, and g are independently 0, 1, 2, 3, or 4.

As described above, in certain embodiments, the present invention provides a compound of formula I-b, wherein DBM is a compound of formula I-b-c:

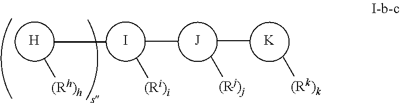

or a pharmaceutically acceptable salt thereof, wherein:
Ring H is a 3-11 membered saturated or partially unsaturated monocyclic, bicyclic, bridged bicyclic, or spirocyclic carbocyclyl or heterocyclyl with 1-3 heteroatoms independently selected from nitrogen, oxygen, and sulfur;
Ring I is phenylenyl or a 5-10 membered monocyclic or bicyclic heteroarylenyl with 1-4 heteroatoms independently selected from nitrogen, oxygen and sulfur;
Ring J is a 3-11 membered saturated or partially unsaturated monocyclic, bicyclic, bridged bicyclic, or spirocyclic carbocyclylenyl or heterocyclylenyl with 1-3 heteroatoms independently selected from nitrogen, oxygen, and sulfur;
Ring K is phenyl, naphthyl, or a 5-13 membered monocyclic, bicyclic, or tricyclic heteroarylenyl with 1-5 heteroatoms independently selected from nitrogen, oxygen and sulfur;
R^{h}, Rⁱ, R^{j}, and R^{k} are each independently selected from hydrogen, oxo, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —OC(O)R, —OC(O)NR₂, —OP(O)R₂, —OP(O)(OR)₂, —OP(O)(OR)NR₂, —OP(O)(NR₂)₂, —NRC(O)OR, —NRC(O)R, —NRC(O)N(R)₂, —NRS(O)₂R, —NP(O)R₂, —NRP(O)(OR)₂, —NRP(O)(OR)NR₂. —NRP(O)(NR₂)₂, —P(O)R₂, —P(O)(OR)₂, —P(O)(OR)NR₂, and —P(O)(NR₂)₂, or:
   an Rⁱ group on Ring I and an R^{j} group or Ring J are optionally taken together with their intervening atoms to form a 5-8 membered saturated, partially unsaturated, or aromatic ring having 0-3 heteroatoms independently selected from nitrogen, oxygen, and sulfur, or:
each of X¹ and X² are independently a covalent bond, spiro-fusion between the two rings that X¹ or X² connect, or a bivalent, saturated or unsaturated, straight or branched C₁₋₆ hydrocarbon chain, wherein 0-4 methylene units of X¹ and X² are independently replaced by —CR₂—, —CR(OR)—, —CRF—, —CF₂—, —C(NR)—, —C(O)—, —O—, —N(R)—, —S—, —S(O)—, or —S(O)₂—;
s″ is 0 or 1; and
each of w, x, y, and z are independently 0, 1, 2, 3, or 4.

In certain embodiments, the present invention provides a compound of formula I-b-c as any one of the following formula:

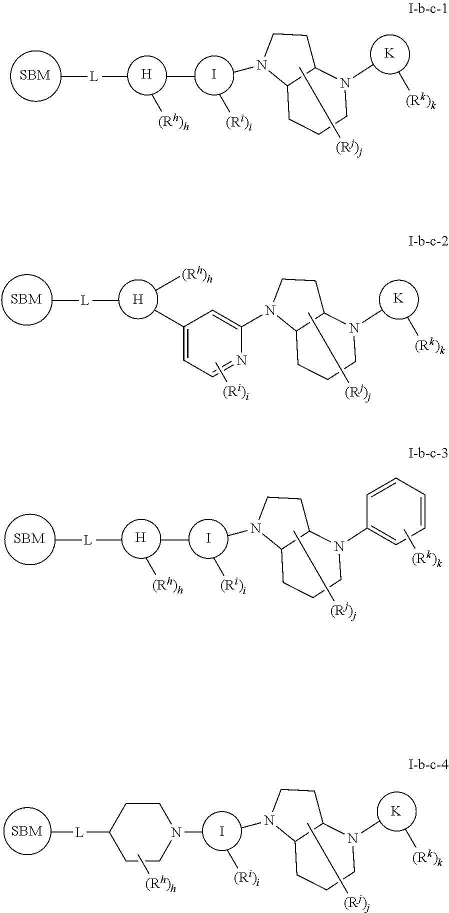

or a pharmaceutically acceptable salt thereof, wherein each of the variables is as defined above in formula I-b-c and described in embodiments herein, both singly and in combination.

In certain embodiments, the present invention provides a compound of formula I-c-a-1 or I-c-a-2 as any one of the following formula:

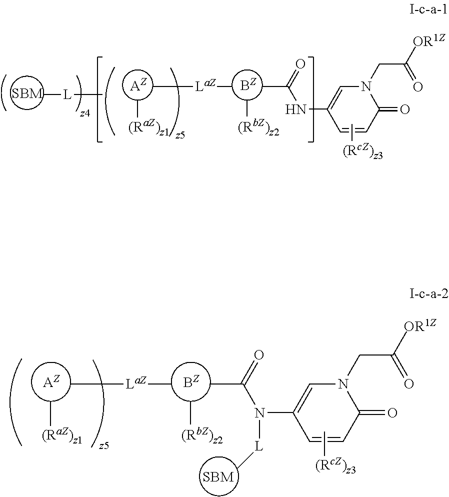

or a pharmaceutically acceptable salt thereof, wherein:
SBM and L are as defined and described above and herein;
R^{1Z} is hydrogen or optionally substituted C₁₋₆ aliphatic;
each R^{aZ}, R^{bZ}, and R^{cZ} are independently hydrogen, R^{AZ}, halogen, —CN, —NO₂, —OR^{Z}—SR^{Z}—NR^{Z}₂, —S(O)₂Rz, —S(O)₂NR^{Z}₂, —S(O)R^{Z}—S(O)(NR^{Z})R^{Z}, —P(O)(OR^{Z})₂, —P(O)(NR^{Z}₂)₂, —CFR^{Z}₂, —CR^{Z}F₂, —CF₃, —CR^{Z}₂(OR^{Z}), —CR₂(NR₂), —C(O)R^{Z}, —C(O)OR^{Z}, or —C(O)NR^{Z}₂;
each R^{AZ} is independently an optionally substituted group selected from C₁₋₁₀ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R^{Z} is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R^{Z} groups on the same atom are optionally taken together with their intervening atom to form an optionally substituted 4-11 membered saturated or partially unsaturated carbocyclic or
heterocyclic monocyclic, bicyclic, bridged bicyclic, spirocyclic, or heteroaryl ring having 0-3 heteroatoms, in addition to the atom to which they are attached, independently selected from nitrogen, oxygen, and sulfur:
each Ring A^{Z} is independently a bivalent ring selected from phenylenyl, naphthylenyl, a 4-10 membered saturated or partially unsaturated monocyclic or bicyclic carbocyclylenyl or heterocyclylenyl having 1-3 heteroatoms independently selected from nitrogen, oxygen, and sulfur, or a 5-10 membered monocyclic or bicyclic heteroarylenyl having 1-4 heteroatoms independently selected from nitrogen, oxygen, and sulfur;
each Ring B^{Z} is independently a bivalent ring selected from phenylenyl, a 3-10 membered saturated or partially unsaturated monocyclic, bicyclic, bridged bicyclic, or spirocyclic carbocyclylenyl or heterocyclylenyl having 1-3 heteroatoms independently selected from nitrogen, oxygen, and sulfur, or a 5-10 membered monocyclic or bicyclic heteroarylenyl having 1-4 heteroatoms independently selected from nitrogen, oxygen, and sulfur;
L^{aZ} is absent, a covalent bond, or a C₁₋₃ bivalent straight or branched saturated or unsaturated hydrocarbon chain, wherein 1-3 methylene units of the chain are independently and optionally replaced with —O—, —C(O)—, —C(S)—, —C(R^{Z})₂—, —CH(R^{Z})—, —CF(R^{Z})—, —C(F)₂, —N(R^{Z})—, —S—, —S(O)₂— or —CR^{Z}═CR^{Z}—:
z1, z2, and z3 are each independently 0, 1, 2, 3 or 4;
each of z4 and z5 is independently 0 or 1.

In some embodiments, a provided compound is of formula I-c-a-1, or a pharmaceutically acceptable salt thereof. In some embodiments, a provided compound is of formula I-c-a-2, or a pharmaceutically acceptable salt thereof.

In some embodiments, R^{1Z} is hydrogen, methyl, or ethyl. In some embodiments, R^{1Z} is hydrogen.

In some embodiments, each Ring Az is independently a bivalent ring selected from phenylenyl, naphthalenyl, or a 5-10 membered monocyclic or bicyclic heteroarylenyl having 1-4 heteroatoms independently selected from nitrogen, oxygen, and sulfur. In some embodiments, each Ring B is independently a bivalent ring selected from phenylenyl, a 5-6 membered saturated or partially unsaturated monocyclic carbocyclylenyl or heterocyclylenyl having 1-3 heteroatoms independently selected from nitrogen, oxygen, and sulfur, or a 5-10 membered monocyclic or bicyclic heteroarylenyl having 1-4 heteroatoms independently selected from nitrogen, oxygen, and sulfur.

In certain embodiments, the present invention provides a compound of formula I, wherein LBM is a CRBN E3 ubiquitin ligase binding moiety thereby forming a compound of formula I-bbbb-1:

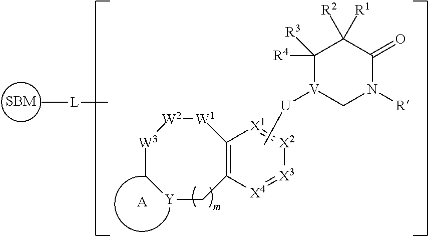

or a pharmaceutically acceptable salt thereof, wherein L and SBM are as defined above and described in embodiments herein, and
each additional variable as described and defined in CN 118005655 A, the entirety of which is herein incorporated by reference.

In certain embodiments, the present invention provides a compound of formula I, wherein LBM is a CRBN E3 ubiquitin ligase binding moiety thereby forming a compound of formula I-cccc-1:

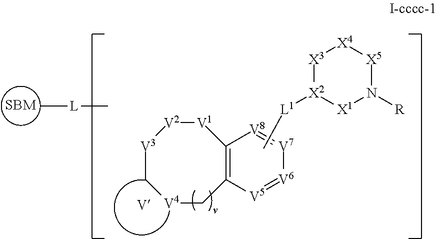

or a pharmaceutically acceptable salt thereof, wherein L, SBM, X¹, X², X³, X⁴, X⁵, R, and L¹ are as defined above and described in embodiments herein, wherein:
One of V⁵, V⁶, V⁷, and V¹ is a carbon atom which is attached to L¹, and the others are independently selected from N or CR^{V};
each R^{V} is independently selected from hydrogen, halogen, —OR, —NR₂, —CN, or an optionally substituted group selected from C₁₋₆ aliphatic, a 3-8 membered saturated or partially unsaturated carbocyclyl or heterocyclyl having 1-3 heteroatom independently selected from nitrogen, oxygen, or sulfur, phenyl, or a 5-6 membered heteroaryl having 1-3 heteroatom independently selected from nitrogen, oxygen, or sulfur;
each of V¹, V², and V³ is independently selected form a bond, —S—, —S(O)—, —S(O)₂—, —C(O)—, —C(R^{V}₂)—, —(C(R^{V}₂))₂—, or —N(R^{V})—,
V⁴ is selected from N, C, or CR; and
Ring V′ is an optionally substituted fused saturated or partially unsaturated carbocyclyl or heterocyclyl having 1-3 heteroatom independently selected from nitrogen, oxygen, or sulfur.

### Degradation Inducing Moiety (DIM)

In certain embodiments, the present invention provides a compound of formula I:

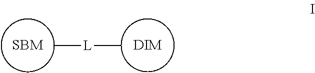

or a pharmaceutically acceptable salt thereof, wherein L and SBM are as described above and herein, and DIM is a degradation inducing moiety selected from LBM, a lysine mimetic, or a hydrogen atom.

In some embodiments, DIM is LBM as described above and herein. In some embodiments, DIM is a lysine mimetic. In some embodiments, the covalent attachment of ubiquitin to STAT6 protein is achieved through the action of a lysine mimetic. In some embodiments, upon the binding of a compound of formula I to STAT6 protein, the moiety that mimics a lysine undergoes ubiquitination thereby marking STAT6 protein for degradation via the Ubiquitin-Proteasome Pathway (UPP).

In some embodiments, DIM is

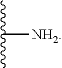 [SMILES: *N]

In some embodiments, DIM is

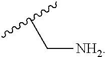 [SMILES: *CN]

In some embodiments, DIM is

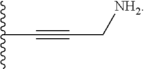 [SMILES: *C#CCN]

In some embodiments, DIM is selected from those depicted in Table 2, below.

In some embodiments, the present invention provides the compound of formula I as a compound of formula I-aaaa:

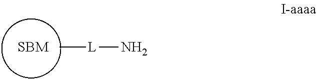

or a pharmaceutically acceptable salt thereof, wherein each of SBM and L is as defined above and described in embodiments herein, both singly and in combination.

In some embodiments, the present invention provides the compound of formula I as a compound of formula I-aaaa-1:

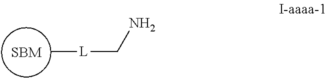

or a pharmaceutically acceptable salt thereof, wherein each of SBM and L is as defined above and described in embodiments herein, both singly and in combination.

In some embodiments, the present invention provides the compound of formula I as a compound of formula I-aaaa-2:

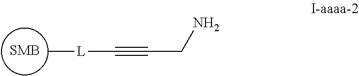

or a pharmaceutically acceptable salt thereof, wherein each of SBM and L is as defined above and described in embodiments herein, both singly and in combination.

In certain embodiments, the present invention provides a compound of Formula I, wherein DIM is a lysine mimetic

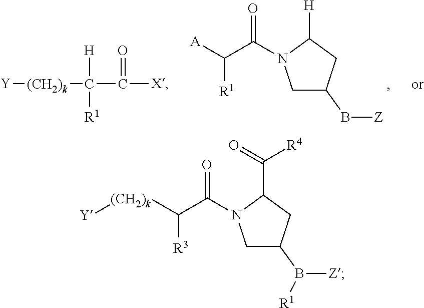

thereby forming a compound of Formulae I-bbbb-1, I-bbbb-2, or I-bbbb-3, respectively:

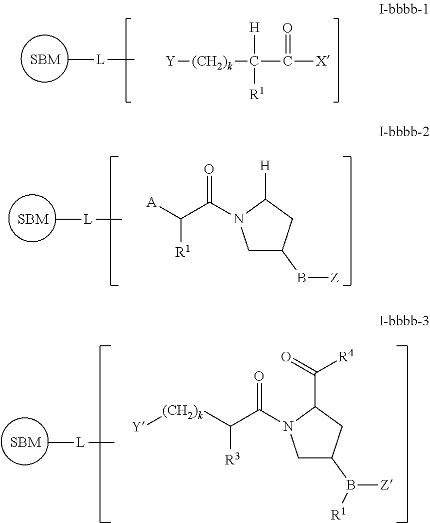

or a pharmaceutically acceptable salt thereof, wherein L and SBM are as defined above and described in embodiments herein, and wherein:
A is (CH₂)ₖ—Y′;
k is 0, 1, or 2;
Y′ is OR² or NR²R³:
R¹ is selected from H, an optionally substituted C₁₋₁₀alkyl, an optionally substituted C₆₋₂₀aryl, an optionally substituted C₇₋₂₀aralkyl, and an amino acid side chain;
alternatively, A and R¹ together with the carbon atom to which they are bound form a 5-20 membered heteroaryl containing 1-4 ring heteroatoms independently selected from N, O, and S and optionally substituted with 1-5 Q groups;
B is selected from NR¹, NR(CH₂)ₙC(O), NR⁵(CH₂)ₙ, S(O)₂, and an amide bioisostere:
n is 0, 1, or 2;
Z is selected from H, (CH₂)m-C₆₋₂₀ aryl optionally substituted with 1-5 Q groups, and (CH₂)ₘ-5-20 membered heteroaryl optionally substituted with 1-5 Q groups;
Z′ is selected from H, (CH₂)ₘ—C₆₋₂₀ aryl, (CH₂)ₘ-5-20 membered heteroaryl, C(O)(CH₂)ₘ—C₆₋₂₀ aryl, C(O)(CH₂)ₘ-5-20 membered heteroaryl, (CH₂)ₘC(O)—C₆₋₂₀ aryl, (CH₂)ₘC(O)-5-20 membered heteroaryl, S(O)₂(CH₂)ₘ—C₆₋₂₀ aryl, and S(O)₂(CH₂)ₘ-5-20 membered heteroaryl, wherein each of the C₆₋₂₀ aryl and 5-20 membered heteroaryl is optionally substituted with 1-5 Q groups;
m is 0, 1, or 2;
E is selected from C(O)OR⁶, C(O)NR⁶R⁷, a carboxylic acid bioisostere and an amide bioisostere;
Q, at each occurrence, independently is selected from an optionally substituted C₁₋₁₀alkyl, an optionally substituted C₂₋₁₀alkenyl, an optionally substituted C₂₋₁₀alkynyl, an optionally substituted C3-20 cycloalkyl, an optionally substituted C₆₋₂₀ aryl, an optionally substituted C₇₋₂₀ aralkyl, an optionally substituted 3-20 membered cycloheteroalkyl, an optionally substituted 5-20 membered heteroaryl, F, Cl, Br, I, CN, CF₃, OCF₃, NO₂, OR^{g}, SR, S⁺R⁸₂, S(O)R⁸, S(O)₂R⁸, S(O)₂OH, S(O)₂NR⁸R⁹, NR⁸S(O)₂R⁹, C(O)R⁸, C(O)OR⁸, C(O)NR⁸R⁹, OC(O)R⁸, NR⁸R⁹, NR⁸C(O)R⁹, NR⁸C(O)OR₉, NR⁸C(O)NR⁸R⁹, and N⁺R⁸₃;
R² and R³ each independently is selected from H, an optionally substituted C₁₋₁₀alkyl, an optionally substituted C₃₋₂₀ cycloalkyl, an optionally substituted C₇-20 aralkyl, an optionally substituted C₆₋₂₀ aryl, an optionally substituted 3-20 membered cycloheteroalkyl, an optionally substituted 5-20 membered heteroaryl, C(O)R⁶, C(O)OR⁶, C(O)NR⁶R⁷, S(O)₂R⁶, and S(O)₂NR⁶R⁷;
alternatively, R² and R³ together with the nitrogen atom to which they are bound form a 3-20 membered heterocycle optionally containing 1-4 ring heteroatoms independently selected from O, N and S atoms and optionally substituted with 1-5 Q groups:
R⁵ is H or an optionally substituted C₁₋₁₀ alkyl;
R⁶ and R⁷ each independently is selected from H, an optionally substituted C₁₋₁₀ alkyl, an optionally substituted C₃₋₂₀ cycloalkyl, an optionally substituted C₂₋₁₀ alkenyl, an optionally substituted C₂₋₁₀ alkynyl, an optionally substituted C₆₋₂₀ aryl, an optionally substituted C₇₋₂₀ aralkyl, an optionally substituted 3-20 membered cycloheteroalkyl, an optionally substituted 5-20 membered heteroaryl, C(O)R⁸, C(O)OR^{B}, and C(O)NR⁸R⁹;
alternatively, R⁶ and R₇ together with the nitrogen atom to which they are bound form a 3-20 membered heterocycle optionally containing 1-4 ring heteroatoms independently selected from O, N and S and optionally substituted with 1-5 Q groups; and
R⁸ and R⁹ each independently is selected from H, an optionally substituted C,-,, alkyl, an optionally substituted C³⁻²⁰ cycloalkyl, an optionally substituted C₂₋₁₀ alkenyl, an optionally substituted C₂₋₁₀ alkynyl, an optionally substituted C₆₋₂₀ aryl, an optionally substituted C₇₋₂₀ aralkyl, an optionally substituted 3-20 membered cycloheteroalkyl, and an optionally substituted 5-20 membered heteroaryl, provided that the compound is not 1-(2-aminopropanoyl)-4-benzamidopyrrolidine-2-carboxylic acid,
as defined and described in U.S. Pat. No. 7,622,496, the entirety of each of which is herein incorporated by reference.

### Linker (L)

As defined above and described herein, L is a bivalent moiety that connects SBM to LBM or SBM to DIM as defined and described herein.

In some embodiments, L is a bivalent moiety that connects SBM to LBM as defined and described herein. In some embodiments, L is a bivalent moiety that connects SBM to DIM as defined and described herein. In some embodiments, L is a bivalent moiety that connects SBM to a lysine mimetic as defined and described herein.

In some embodiments, L is a covalent bond or an optionally substituted bivalent, saturated or partially unsaturated, straight or branched C₁₋₂₀ hydrocarbon chain, wherein 0-6 methylene units of L are independently replaced by -Cy-, —CHF—, —CF₂—, —O—, —NR—, —SiR₂—, —Si(OH)R—, —Si(OH)₂—, —P(O)OR—, —P(O)R—, —P(O)NR₂—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

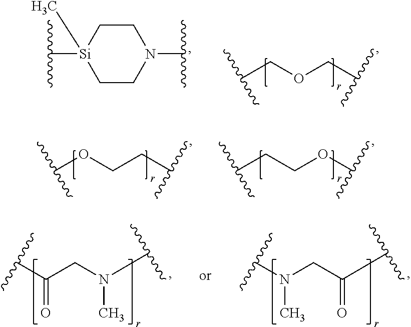

wherein:
each -Cy- is independently an optionally substituted bivalent ring selected from phenylenyl, an 8-10 membered bicyclic arylenyl, a 3-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur,
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur, and;
each r is independently 0, 1, 2, 3, 4, 5, 6, 7, 8, 9, or 10.

In some embodiments, L is a covalent bond or an optionally substituted bivalent, saturated or partially unsaturated, straight or branched C₁₋₂₀ hydrocarbon chain, wherein 0-6 methylene units of L are independently replaced by -Cy-, —CHF—, —CF₂—, —O—, —NR—, —SiR₂—, —Si(OH)R—, —Si(OH)₂—, —P(O)OR—, —P(O)R—, —P(O)NR₂—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

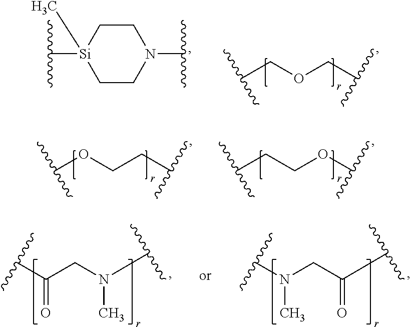

wherein:
each -Cy- is independently an optionally substituted bivalent ring selected from phenylenyl, an 8-10 membered bicyclic arylenyl, a 4-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur,
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur, and;
each r is independently 0, 1, 2, 3, 4, 5, 6, 7, 8, 9, or 10.

In some embodiments, L is a covalent bond or a bivalent, saturated or partially unsaturated, straight or branched C₁₋₂₀ hydrocarbon chain, wherein 0-6 methylene units of L are independently replaced by -Cy-, —CHF—, —CF₂—, —O—, —NR—, —SiR₂—, —Si(OH)R—, —Si(OH)₂—, —P(O)OR—, —P(O)R—, —P(O)NR₂—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —C(O)NR—, —OC(O)NR—, —NRC(O)O—,

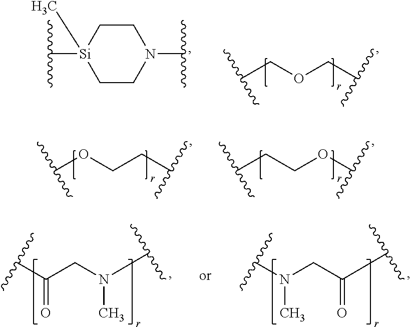

wherein:
each -Cy- is independently an optionally substituted bivalent ring selected from phenylenyl, an 8-10 membered bicyclic arylenyl, a 4-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur,
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the sane nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur, and;
each r is independently 0, 1, 2, 3, 4, 5, 6, 7, 8, 9, or 10.

In some embodiments, L is a covalent bond or an optionally substituted bivalent, saturated or partially unsaturated, straight or branched C₁₋₂₀ hydrocarbon chain, wherein 0-4 methylene units of L are independently replaced by -Cy-, —CHF—, —CF₂—, —O—, —NR—, —SiR₂—, —Si(OH)R—, —Si(OH)₂—, —P(O)OR—, —P(O)R—, —P(O)NR₂—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

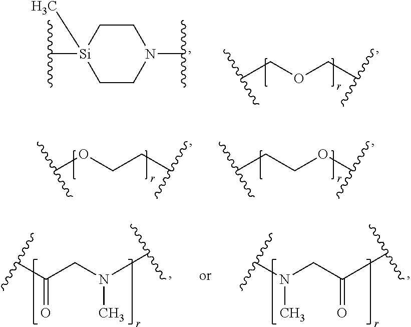

wherein:
each -Cy- is independently an optionally substituted bivalent ring selected from phenylenyl, an 8-10 membered bicyclic arylenyl, a 3-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur,
each R is independently hydrogen, or an optionally substituted group selected from Cv- aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur, and;
each r is independently 0, 1, 2, 3, 4, 5, 6, 7, 8, 9, or 10.

In some embodiments, L is a covalent bond. In some embodiments, L is a bivalent, saturated or partially unsaturated, straight or branched C₁₋₂₀ or C₁₋₁₀ hydrocarbon chain, wherein 0-6 methylene units of L are independently replaced by -Cy-, —CHF—, —CF₂—, —O—, —NR—, —SiR₂—, —Si(OH)R—, —Si(OH)₂—, —P(O)OR—, —P(O)R—, —P(O)NR₂—, —S—, —OC(O)—, —C(O)O—, —C(O)—, —S(O)—, —S(O)₂— —NRS(O)₂—, —S(O)₂NR—. —NRC(O)—, —C(O)NR—, —OC(O)NR—, —NRC(O)O—,

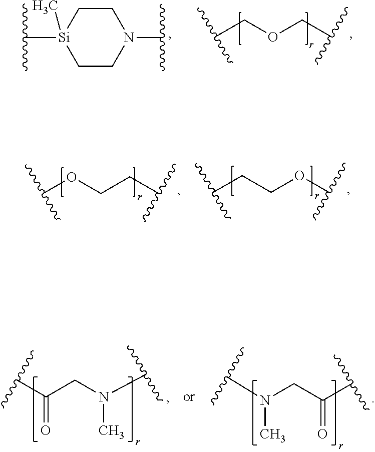

In some embodiments, L is a bivalent, saturated or partially unsaturated, straight or branched C₁₋₂₀ hydrocarbon chain, wherein 0-6 methylene units of L are independently replaced by -Cy-, —CHF—, —CF₂—, —O—, —NR—, —S—, —C(O)O—, —C(O)—, —S(O)—, —S(O)₂—, —NRS(O)₂—, —NRC(O)—, —NRC(O)O—,

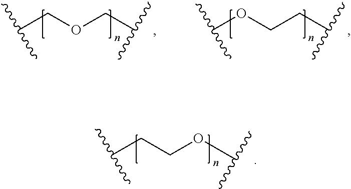

In some embodiments, L is an optionally substituted bivalent, saturated or partially unsaturated, straight or branched C₁₋₁₀ hydrocarbon chain, wherein 0-3 methylene units of L are independently replaced by -Cy-, —CHF—, —CF₂—, —O—, —NR—, —SiR₂—, —Si(OH)R—, —Si(OH)₂—, —P(O)OR—, —P(O)R—, —P(O)NR₂—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

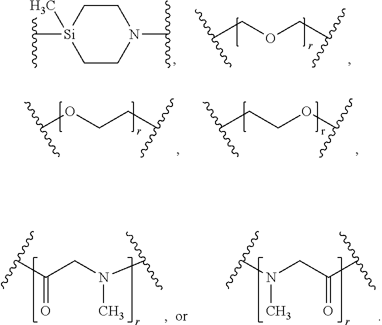

In some embodiments, L is an optionally substituted bivalent, saturated or partially unsaturated, straight or branched C₁₋₁₀ hydrocarbon chain, wherein 0-3 methylene units of L are independently replaced by -Cy-, —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is a bivalent, saturated or partially unsaturated, straight or branched C₁₋₁₀ hydrocarbon chain, wherein 0-3 methylene units of L are independently replaced by -Cy-, —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O, wherein L is optionally substituted with halogen or —R^{∘}. In some embodiments, L is an optionally substituted bivalent, saturated or partially unsaturated, straight or branched C₁₋₁₀ hydrocarbon chain, wherein 1-3 methylene units of L are independently replaced by -Cy-, and 1-2 additional methylene units of L are optionally and independently replaced with —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O.

In some embodiments, L is an optionally substituted bivalent, saturated or partially unsaturated, straight or branched C₁₋₅ hydrocarbon chain, wherein 0-3 methylene units of L are independently replaced by -Cy-, —CHF—, —CF₂—, —O—, —NR—, —SiR₂—, —Si(OH)R—, —Si(OH)₂—, —P(O)OR—, —P(O)R—, —P(O)NR₂—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

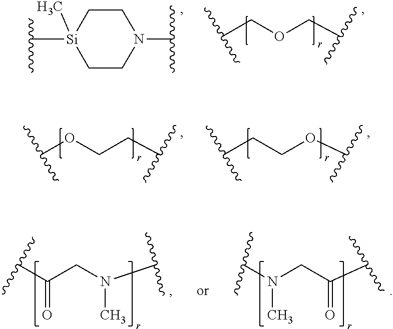

In some embodiments, L is an optionally substituted bivalent, saturated or partially unsaturated, straight or branched C₁₋₅ hydrocarbon chain, wherein 0-3 methylene units of L are independently replaced by -Cy-, —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is a bivalent, saturated or partially unsaturated, straight or branched C₁₋₅ hydrocarbon chain, wherein 0-3 methylene units of L are independently replaced by -Cy-, —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O, wherein L is optionally substituted with halogen or —R^{∘}. In some embodiments, L is an optionally substituted bivalent, saturated or partially unsaturated, straight or branched C₁₋₅ hydrocarbon chain, wherein 1-3 methylene units of L are independently replaced by -Cy-, and 1-2 additional methylene units of L are optionally and independently replaced with —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O.

In some embodiments, L is an optionally substituted bivalent, saturated or partially unsaturated, straight or branched C₁₋₃ hydrocarbon chain, wherein 0-3 methylene units of L are independently replaced by -Cy-, —CHF—, —CF₂—, —O—, —NR—, —SiR₂—, —Si(OH)R—, —Si(OH)₂—, —P(O)OR—, —P(O)R—, —P(O)NR₂—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NC(O)O—,

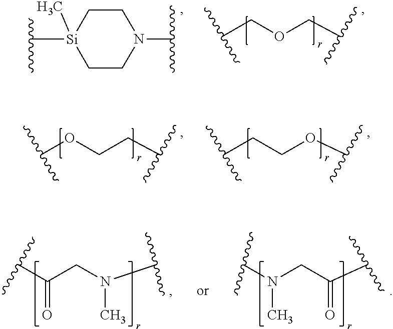

In some embodiments, L is an optionally substituted bivalent, saturated or partially unsaturated, straight or branched C₁₋₃ hydrocarbon chain, wherein 0-3 methylene units of L are independently replaced by -Cy-, —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is a bivalent, saturated or partially unsaturated, straight or branched C₁₋₃ hydrocarbon chain, wherein 0-3 methylene units of L are independently replaced by -Cy-, —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O, wherein L is optionally substituted with halogen or —R^{∘}. In some embodiments, L is an optionally substituted bivalent, saturated or partially unsaturated, straight or branched C₁₋₃ hydrocarbon chain, wherein 1-3 methylene units of L are independently replaced by -Cy-, and 1-2 additional methylene units of L are optionally and independently replaced with —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O.

In some embodiments, L is an optionally substituted bivalent, saturated or partially unsaturated, straight or branched C₁₋₂ hydrocarbon chain, wherein 0-3 methylene units of L are independently replaced by -Cy-, —CHF—, —CF₂—, —O—, —NR—, —SiR₂—, —Si(OH)R—, —Si(OH)₂—, —P(O)OR—, —P(O)R—, —P(O)NR₂—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

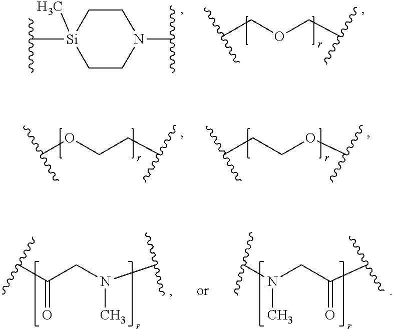

In some embodiments, L is an optionally substituted bivalent, saturated or partially unsaturated, straight or branched C₁₋₂ hydrocarbon chain, wherein 0-3 methylene units of L are independently replaced by -Cy-, —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is a bivalent, saturated or partially unsaturated, straight or branched C₁₋₂ hydrocarbon chain, wherein 0-3 methylene units of L are independently replaced by -Cy-, —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O, wherein L is optionally substituted with halogen or —R^{∘}. In some embodiments, L is an optionally substituted bivalent, saturated or partially unsaturated, straight or branched C₁₋₂ hydrocarbon chain, wherein 1-3 methylene units of L are independently replaced by -Cy-, and 1-2 additional methylene units of L are optionally and independently replaced with —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O.

In some embodiments, L is a bivalent, saturated or partially unsaturated, straight or branched C₁₋₂₀ hydrocarbon chain, wherein 0-6 methylene units of L are independently replaced by -Cy-, —CHF—, —CF₂—, —O—, —NR—, —S—, —C(O)O—, —C(O)—, —S(O)—, —S(O)₂—, —NRS(O)₂—, —NRC(O)—, or NRC(O)O—.

In some embodiments, L is a bivalent, saturated or partially unsaturated, straight or branched C₁₋₁₀ hydrocarbon chain, wherein 0-6 methylene units of L are independently replaced by -Cy-, —CHF—, —CF₂—, —O—, —NR—, —S—, —C(O)O—, —C(O)—, —S(O)—, —S(O)₂—, —NRS(O)₂—, —NRC(O)—, or NRC(O)O—.

In some embodiments, L is a bivalent, saturated or partially unsaturated, straight or branched C₁ₗo hydrocarbon chain, wherein 0-6 methylene units of L are independently replaced by -Cy-, —CHF—, —CF₂—, —O—, —NR—, —S—, —C(O)O—, —C(O)—, —S(O)—, —S(O)₂—, —NRS(O)₂—, or NRC(O)O—, provided that L does not comprise —NR—C(O)—.

In some embodiments, L is a bivalent, saturated or partially unsaturated, straight or branched C₁₋₁₀ hydrocarbon chain, wherein 0-6 methylene units of L are independently replaced by -Cy-, —CHF—, —CF₂—, —O—, —S—, —C(O)O—, —C(O)—, —S(O)—, —S(O)₂—, —NRS(O)₂—, —NRC(O)—, or NRC(O)O—.

In some embodiments, Lis —NR—(C₁₋₁₀aliphatic)-. In some embodiments, Lis —(C₁₋₁₀aliphatic)-NR—(C₁₋₁₀aliphatic)-. In some embodiments, L is —(C₁₋₁₀ aliphatic)-NR—(CH₂CH₂O)₁₋₁₀CH₂CH₂—. In some embodiments, L is -Cy-NR—(C₁₋₁₀ aliphatic)-. In some embodiments, L is -Cy-(C₁₋₁₀ aliphatic)-NR—. In some embodiments, L is -Cy-(C₁₋₁₀ aliphatic)-NR—(C₁₋₁₀ aliphatic)-. In some embodiments, L is —(C₁₋₁₀ aliphatic)-Cy-NR—(C₁₋₁₀ aliphatic)-. In some embodiments, L is —(C₁₋₁₀ aliphatic)-Cy-(C₁₋₁₀ aliphatic)-NR—. In some embodiments, L is —(C₁₋₁₀ aliphatic)-Cy-(C₁₋₁₀ aliphatic)-NR—(C₁₋₁₀ aliphatic)-. In some embodiments, L is -Cy-(C₁₋₁₀ aliphatic)-Cy-NR—. In some embodiments, L is -Cy-(C₁₋₁₀ aliphatic)-NR-Cy-. In some embodiments, L is -Cy-(C₁₋₁₀ aliphatic)-Cy-NR—(C₁₋₁₀ aliphatic)-. In some embodiments, L is -Cy-(C₁₋₁₀ aliphatic)-NR-Cy-(C₁₋₁₀ aliphatic)-.

In some embodiments, L is —CONR—(C₁₋₁₀ aliphatic)-. In some embodiments, L is —(C₁₋₁₀ aliphatic)-CONR—(C₁₋₁₀aliphatic)-. In some embodiments, L is —(C₁₋₁₀ aliphatic)-CONR—(CH₂CH₂O)₁₋₁₀CH₂CH₂—. In some embodiments, L is -Cy-CONR—(C₁₋₁₀aliphatic)-. In some embodiments, L is -Cy-(C₁₋₁₀ aliphatic)-CONR—. In some embodiments, L is -Cy-(C₁₋₁₀ aliphatic)-CONR—(C₁₋₁₀ aliphatic)-. In some embodiments, L is —(C₁₋₁₀ aliphatic)-Cy-CONR—(C₁₋₁₀ aliphatic)-. In some embodiments, L is —(C₁₋₁₀ aliphatic)-Cy-(C₁₋₁₀ aliphatic)-CONR—. In some embodiments, L is —(C₁₋₁₀ aliphatic)-Cy-(C₁₋₁₀ aliphatic)-CONR—(C₁₋₁₀ aliphatic)-. In some embodiments, L is -Cy-(C₁₋₁₀ aliphatic)-Cy-CONR—. In some embodiments, L is -Cy-(C₁₋₁₀ aliphatic)-CONR-Cy-. In some embodiments, L is -Cy-(C₁₋₁₀ aliphatic)-Cy-CONR—(C₁₋₁₀ aliphatic)-. In some embodiments, L is -Cy-(C₁₋₁₀ aliphatic)-CONR-Cy-(C₁₋₁₀ aliphatic)-.

In some embodiments, L is —NRCO—(C₁₋₁₀ aliphatic)-. In some embodiments, L is —(C₁₋₁₀ aliphatic)-NRCO—(C₁₋₁₀aliphatic)-. In some embodiments, L is —(C₁₋₁₀ aliphatic)-NRCO—(CH₂CH₂O)₁₋₁₀CH₂CH₂—. In some embodiments, L is -Cy-NRCO—(C₁₋₁₀ aliphatic)-. In some embodiments, L is -Cy-(C₁₋₁₀ aliphatic)-NRCO—. In some embodiments, L is -Cy-(C₁₋₁₀ aliphatic)-NRCO—(C₁₋₁₀ aliphatic)-. In some embodiments, L is —(C₁₋₁₀ aliphatic)-Cy-NRCO—(C₁₋₁₀ aliphatic)-. In some embodiments, L is —(C₁₋₁₀ aliphatic)-Cy-(C₁₋₁₀ aliphatic)-NRCO—. In some embodiments, L is —(C₁₋₁₀ aliphatic)-Cy-(C₁₋₁₀ aliphatic)-NRCO—(C₁₋₁₀ aliphatic)-. In some embodiments, L is -Cy-(C₁₋₁₀ aliphatic)-Cy-NRCO—. In some embodiments, L is -Cy-(C₁₋₁₀ aliphatic)-NRCO-Cy-. In some embodiments, L is -Cy-(C₁₋₁₀ aliphatic)-Cy-NRCO—(C₁₋₁₀ aliphatic)-. In some embodiments, L is -Cy-(C₁₋₁₀ aliphatic)-NRCO-Cy-(C₁₋₁₀ aliphatic)-.

In some embodiments, L is —O—(C₁₋₁₀ aliphatic)-. In some embodiments, L is —(C₁₋₁₀ aliphatic)-O—(C₁₋₁₀aliphatic)-. In some embodiments, L is —(C₁₋₁₀ aliphatic)-O—(CH₂CH₂O)₁₋₁₀CH₂CH₂—. In some embodiments, L is -Cy-O—(C₁₋₁₀ aliphatic)-. In some embodiments, L is -Cy-(C₁₋₁₀ aliphatic)-O—. In some embodiments, L is -Cy-(C₁₋₁₀ aliphatic)-O—(C₁₋₁₀ aliphatic)-. In some embodiments, L is —(C₁₋₁₀ aliphatic)-Cy-O—(C₁₋₁₀ aliphatic)-. In some embodiments, L is —(C₁₋₁₀ aliphatic)-Cy-(C₁₋₁₀ aliphatic)-O—. In some embodiments, L is —(C₁₋₁₀ aliphatic)-Cy-(C₁₋₁₀ aliphatic)-O—(C₁₋₁₀ aliphatic)-. In some embodiments, L is —Cy-(C₁₋₁₀aliphatic)-Cy-O—. In some embodiments, L is -Cy-(C₁₋₁₀ aliphatic)-O-Cy-. In some embodiments, L is -Cy-(C₁₋₁₀ aliphatic)-Cy-O—(C₁₋₁₀ aliphatic)-. In some embodiments, L is -Cy-(C₁₋₁₀ aliphatic)-O-Cy-(C₁₋₁₀ aliphatic)-.

In some embodiments, L is —(C₁₋₁₀ aliphatic)-. In some embodiments, L is -Cy-(C₁₋₁₀ aliphatic)-. In some embodiments, L is —(C₁₋₁₀ aliphatic)-Cy-(C₁₋₁₀ aliphatic)-. In some embodiments, L is —(C₁₋₁₀ aliphatic)-Cy-(CH₂CH₂O)₁₋₁₀CH₂CH₂—. In some embodiments, L is -Cy-(C₁₋₁₀ aliphatic)-Cy-. In some embodiments, L is -Cy-(C₁₋₁₀ aliphatic)-Cy-(C₁₋₁₀ aliphatic)-. In some embodiments, L is -Cy-(C₁₋₁₀ aliphatic)-Cy-(C₁₋₁₀ aliphatic)-Cy-. In some embodiments, L is —(C₁₋₁₀ aliphatic)-Cy-(C₁₋₁₀ aliphatic)-Cy-(C₁₋₁₀ aliphatic)-.

In some embodiments, L is -Cy-(optionally substituted C₁₋₁₀ aliphatic)-. In some embodiments, L is -Cy-(optionally substituted C₁₋₁₀ aliphatic)-Cy-. In some embodiments, L is -Cy-Cy-(optionally substituted C₁₋₁₀ aliphatic)-. In some embodiments, L is -Cy-Cy-Cy-(optionally substituted C₁₋₁₀ aliphatic)-. In some embodiments, L is -Cy-Cy-(optionally substituted C₁₋₁₀ aliphatic)-Cy-. In some embodiments, L is -Cy-(C₁₋₁₀ aliphatic)-. In some embodiments, L is -Cy-Cy-(C₁₋₁₀ aliphatic)-. In some embodiments, L is -Cy-Cy-Cy-(C₁₋₁₀ aliphatic)-. In some embodiments, L is -Cy-Cy-(C₁₋₁₀ aliphatic)-Cy-.

In some embodiments, L is -Cy-(optionally substituted C₁₋₆ aliphatic)-. In some embodiments, L is -Cy-(optionally substituted C₁₋₆ aliphatic)-Cy-. In some embodiments, L is -Cy-Cy-(optionally substituted C₁₋₆ aliphatic)-. In some embodiments, L is -Cy-Cy-Cy-(optionally substituted C₁₋₆ aliphatic)-. In some embodiments, L is -Cy-Cy-(optionally substituted C₁₋₆ aliphatic)-Cy-. In some embodiments, L is -Cy-(C—₆ aliphatic)-. In some embodiments, L is -Cy-(C₁₋₆ aliphatic)-Cy-. In some embodiments, L is -Cy-Cy-(C—₆ aliphatic)-. In some embodiments, L is -Cy-Cy-Cy-(C₁₋₆ aliphatic)-. In some embodiments, L is -Cy-Cy-(C—₆ aliphatic)-Cy-.

In some embodiments, L is -Cy-(optionally substituted C₁₋₃ aliphatic)-. In some embodiments, L is -Cy-(optionally substituted C₁₋₃ aliphatic)-Cy-. In some embodiments, L is -Cy-Cy-(optionally substituted C₁₋₃ aliphatic)-. In some embodiments, L is -Cy-Cy-Cy-(optionally substituted C₁₋₃ aliphatic)-. In some embodiments, L is -Cy-Cy-(optionally substituted C₁₋₃ aliphatic)-Cy-. In some embodiments, L is -Cy-(C₁₋₃ aliphatic)-. In some embodiments, L is -Cy-(C—₃ aliphatic)-Cy-. In some embodiments, L is -Cy-Cy-(C₁₋₃ aliphatic)-. In some embodiments, L is -Cy-Cy-Cy-(C₁₋₃ aliphatic)-. In some embodiments, L is -Cy-Cy-(C—₃ aliphatic)-Cy-.

In some embodiments, L is —NR—(CH₂)₁₋₁₀. In some embodiments, L is —(CH₂)₁₋₁₀—NR—(CH₂)₁₋₁₀—. In some embodiments, L is —(CH₂)₁₋₁₀—NR—(CH₂CH₂O)₁₋₁₀CH₂CH₂—. In some embodiments, L is -Cy-NR—(CH₂)₁₋₁₀—. In some embodiments, L is -Cy-(CH₂)₁₋₁₀—NR—. In some embodiments, L is -Cy-(CH₂)₁₋₁₀—NR—(CH₂)₁₋₁₀—. In some embodiments, L is —(CH₂)₁₋₁₀-Cy-NR—(CH₂)₁₋₁₀. In some embodiments, L is —(CH₂)₁₋₁₀-Cy-(CH₂)₁₋₁₀—NR—. In some embodiments, L is —(CH₂)₁₋₁₀-Cy-(CH₂)₁₋₁₀—NR—(CH₂)₁₋₁₀—. In some embodiments, L is -Cy-(CH₂)₁₋₁₀-Cy-NR—. In some embodiments, L is -Cy-(CH₂)₁₋₁₀—NR-Cy-. In some embodiments, L is -Cy-(CH₂)₁₋₁₀-Cy-NR—(CH₂)₁₋₁₀—. In some embodiments, L is -Cy-(CH₂)₁₋₁₀—NR-Cy-(CH₂)₁₋₁₀—.

In some embodiments, L is —CONR—(CH₂)₁₋₁₀—. In some embodiments, L is —(CH₂)₁₋₁₀—CONR—(CH₂)₁₋₁₀—. In some embodiments, L is —(CH₂)₁₋₁₀—CONR—(CH₂CH₂O)₁₋₁₀CH₂CH₂—. In some embodiments, L is -Cy-CONR—(CH₂)₁₋₁₀—. In some embodiments, L is -Cy-(CH₂)₁₋₁₀—CONR—. In some embodiments, L is -Cy-(CH₂)₁₋₁₀—CONR—(CH₂)₁₋₁₀—. In some embodiments, L is —(CH₂)₁₋₁₀-Cy-CONR—(CH₂)₁₋₁₀—. In some embodiments, L is —(CH₂)₁₋₁₀-Cy-(CH₂)₁₋₁₀—CONR—. In some embodiments, L is —(CH₂)₁₋₁₀-Cy-(CH₂)₁₋₁₀—CONR—(CH₂)₁₋₁₀—. In some embodiments, L is -Cy-(CH₂)₁₋₁₀-Cy-CONR—. In some embodiments, L is -Cy-(CH₂)₁₋₁₀—CONR-Cy-. In some embodiments, L is -Cy-(CH₂)₁₋₁₀-Cy-CONR—(CH₂)₁₋₁₀—. In some embodiments, L is -Cy-(CH₂)₁₋₁₀—CONR-Cy-(CH₂)₁₋₁₀—.

In some embodiments, L is —NRCO—(CH₂)₁₋₁₀—. In some embodiments, L is —(CH₂)₁₋₁₀—NRCO—(CH₂)₁₋₁₀—. In some embodiments, L is —(CH₂)₁₋₁₀—NRCO—(CH₂CH₂O)₁₋₁₀CH₂CH₂—. In some embodiments, L is -Cy-NRCO—(CH₂)₁₋₁₀—. In some embodiments, L is -Cy-(CH₂)₁₋₁₀—NRCO—. In some embodiments, L is -Cy-(CH₂)₁₋₁₀—NRCO—(CH₂)₁₋₁₀—. In some embodiments, L is —(CH₂)₁₋₁₀-Cy-NRCO—(CH₂)₁₋₁₀—. In some embodiments, L is —(CH₂)₁₋₁₀-Cy-(CH₂)₁₋₁₀—NRCO—. In some embodiments, L is —(CH₂)₁₋₁₀-Cy-(CH₂)₁₋₁₀—NRCO—(CH₂)₁₋₁₀—. In some embodiments, L is -Cy-(CH₂)₁₋₁₀-Cy-NRCO—. In some embodiments, L is -Cy-(CH₂)₁₋₁₀—NRCO-Cy-. In some embodiments, L is -Cy-(CH₂)₁₋₁₀-Cy-NRCO—(CH₂)₁₋₁₀—. In some embodiments, L is -Cy-(CH₂)₁₋₁₀—NRCO-Cy-(CH₂)₁₋₁₀—.

In some embodiments, L is —O—(CH₂)₁₋₁₀—. In some embodiments, L is —(CH₂)₁₋₁₀—O—(CH₂)₁₋₁₀—. In some embodiments, L is —(CH₂)₁₋₁₀—O—(CH₂CH₂O)₁₋₁₀CH₂CH₂—. In some embodiments, L is -Cy-O—(CH₂)₁₋₁₀—. In some embodiments, L is -Cy-(CH₂)₁₋₁₀—O—. In some embodiments, L is -Cy-(CH₂)₁₋₁₀—O—(CH₂)₁₋₁₀—. In some embodiments, L is —(CH₂)₁₋₁₀-Cy-O—(CH₂)₁₋₁₀—. In some embodiments, L is —(CH₂)₁₋₁₀—Cy-(CH₂)₁₋₁₀—O—. In some embodiments, L is —(CH₂)₁₋₁₀-Cy-(CH₂)₁₋₁₀—O—(CH₂)₁₋₁₀—. In some embodiments, L is -Cy-(CH₂)₁₋₁₀-Cy-O—. In some embodiments, L is -Cy-(CH₂)₁₋₁₀—O-Cy-. In some embodiments, L is —Cy-(CH₂)₁₋₁₀-Cy-O—(CH₂)₁₋₁₀—. In some embodiments, L is -Cy-(CH₂)₁₋₁₀—O-Cy-(CH₂)₁₋₁₀—.

In some embodiments, L is -Cy-(CH₂)₁₋₁₀—. In some embodiments, L is —(CH₂)₁₋₁₀-Cy-(CH₂)₁₋₁₀—. In some embodiments, L is —(CH₂)₁₋₁₀-Cy-(CH₂CH₂O)₁₋₁₀CH₂CH₂—. In some embodiments, L is -Cy-(CH₂)₁₋₁₀-Cy-. In some embodiments, L is -Cy-(CH₂)₁₋₁₀-Cy-(CH₂)₁₋₁₀—. In some embodiments, L is -Cy-(CH₂)₁₋₁₀-Cy-(CH₂)₁₋₁₀-Cy-. In some embodiments, L is —(CH₂)₁₋₁₀-Cy-(CH₂)₁₋₁₀-Cy-(CH₂)₁₋₁₀—. In some embodiments, L is -Cy-Cy-. In some embodiments, L is -Cy-Cy-(CH₂)₁₋₁₀—. In some embodiments, L is -Cy-(CH₂)₁₋₁₀-Cy-(CH₂)₁₋₁₀—. In some embodiments, L is -Cy-Cy-Cy-. In some embodiments, L is -Cy-Cy-(CH₂)₁₋₁₀-Cy-. In some embodiments, L is -Cy-Cy-(CH₂)₁₋₁₀-Cy-(CH₂)₁₋₁₀—.

In some embodiments, L is optionally substituted -Cy-(CH₂)₁₋₁₀—. In some embodiments, L is optionally substituted —(CH₂)₁₋₁₀-Cy-(CH₂)₁₋₁₀—. In some embodiments, L is optionally substituted —(CH₂)₁₋₁₀-Cy-(CH₂CH₂O)₁₋₁₀CH₂CH₂—. In some embodiments, L is optionally substituted -Cy-(CH₂)₁₋₁₀-Cy-. In some embodiments, L is optionally substituted -Cy-(CH₂)₁₋₁₀-Cy-(CH₂)₁₋₁₀—. In some embodiments, L is optionally substituted -Cy-(CH₂)₁₋₁₀-Cy-(CH₂)₁₋₁₀-Cy-. In some embodiments, L is optionally substituted —(CH₂)₁₋₁₀-Cy-(CH₂)₁₋₁₀-Cy-(CH₂)₁₋₁₀—. In some embodiments, L is optionally substituted -Cy-Cy-. In some embodiments, L is optionally substituted -Cy-Cy-(CH₂)₁₋₁₀—. In some embodiments, L is optionally substituted -Cy-(CH₂)₁₋₁₀-Cy-(CH₂)₁₋₁₀—. In some embodiments, L is optionally substituted -Cy-Cy-Cy-. In some embodiments, L is optionally substituted -Cy-Cy-(CH₂)₁₋₁₀-Cy-. In some embodiments, L is optionally substituted -Cy-Cy-(CH₂)₁₋₁₀-Cy-(CH₂)₁₋₁₀—.

In some embodiments, L comprises one -Cy-group. In some embodiments, L comprises two -Cy- groups. In some embodiments, L comprises three -Cy- groups. In some embodiments, L does not comprise a -Cy- group.

In some embodiments, L comprises —C(O)—.

In some embodiments, L comprises one -Cy- group and one methylene group. In some embodiments, L comprises two -Cy- groups and one methylene group. In some embodiments, L comprises one -Cy- group and two methylene groups.

In some embodiments, L is an optionally substituted bivalent, saturated or partially unsaturated, straight or branched C₁₋₁₀ hydrocarbon chain, wherein 1-4 methylene units of L are independently replaced by -Cy-, and 1-2 additional methylene units of L are optionally and independently replaced with —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is an optionally substituted bivalent, saturated or partially unsaturated, straight or branched C₁₋₁₀ hydrocarbon chain, wherein 2-3 methylene units of L are independently replaced by -Cy-, and 1-2 additional methylene units of L are optionally and independently replaced with —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is an optionally substituted bivalent, saturated or partially unsaturated, straight or branched C₁₋₁₀ hydrocarbon chain, wherein 0-2 methylene units of L are independently replaced with —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is an optionally substituted bivalent, saturated or partially unsaturated, straight or branched C₁₋₁₀ hydrocarbon chain, wherein 1 methylene unit of L is replaced by -Cy-, and 1-2 additional methylene units of L are optionally and independently replaced with —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is an optionally substituted bivalent, saturated or partially unsaturated, straight or branched C₁₋₁₀ hydrocarbon chain, wherein 2 methylene units of L are independently replaced by -Cy-, and 1-2 additional methylene units of L are optionally and independently replaced with —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is an optionally substituted bivalent, saturated or partially unsaturated, straight or branched C₁₋₁₀ hydrocarbon chain, wherein 3 methylene units of L are independently replaced by -Cy-, and 1-2 additional methylene units of L are optionally and independently replaced with —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is an optionally substituted bivalent, saturated or partially unsaturated, straight or branched C₁₋₁₀ hydrocarbon chain, wherein 1 methylene unit of L is replaced by —C(O)—, and 1-3 additional methylene units of L are optionally and independently replaced with -Cy-, —CHF—, —CF₂—, —O—, —NR—, —S—, —OC(O)—, —C(O)O—, —S(O)—, —S(O)₂—, —NRS(O)₂—, —S(O)₂NR—, —NRC(O)—, —C(O)NR—, —OC(O)NR—, or —NRC(O)O.

In some embodiments, L is an optionally substituted bivalent, saturated or partially unsaturated, straight or branched C₁₋₅ hydrocarbon chain, wherein 1-3 methylene units of L are independently replaced by -Cy-, and 1-2 additional methylene units of L are optionally and independently replaced with —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is an optionally substituted bivalent, saturated or partially unsaturated, straight or branched C₁₋₅ hydrocarbon chain, wherein 2-3 methylene units of L are independently replaced by -Cy-, and 1-2 additional methylene units of L are optionally and independently replaced with —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is an optionally substituted bivalent, saturated or partially unsaturated, straight or branched C₁₋₅ hydrocarbon chain, wherein 0-2 methylene units of L are independently replaced with —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is an optionally substituted bivalent, saturated or partially unsaturated, straight or branched C₁₋₅ hydrocarbon chain, wherein 1 methylene unit of L is replaced by —Cy-, and 1-2 additional methylene units of L are optionally and independently replaced with —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is an optionally substituted bivalent, saturated or partially unsaturated, straight or branched C₁₋₅ hydrocarbon chain, wherein 2 methylene units of L are independently replaced by -Cy-, and 1-2 additional methylene units of L are optionally and independently replaced with —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is an optionally substituted bivalent, saturated or partially unsaturated, straight or branched C₁₋₅ hydrocarbon chain, wherein 3 methylene units of L are independently replaced by -Cy-, and 1-2 additional methylene units of L are optionally and independently replaced with —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is an optionally substituted bivalent, saturated or partially unsaturated, straight or branched C₁₋₅ hydrocarbon chain, wherein 1 methylene unit of L is replaced by —C(O)—, and 1-3 additional methylene units of L are optionally and independently replaced with -Cy-, —CHF—, —CF₂—, —O—, —NR—, —S—, —OC(O)—. —C(O)O—, —S(O)—, —S(O)₂—, —NRS(O)₂—, —S(O)₂NR—, —NRC(O)—, —C(O)NR—, —OC(O)NR—, or —NRC(O)O.

In some embodiments, L is an optionally substituted bivalent, saturated or partially unsaturated, straight or branched C₁₋₃ hydrocarbon chain, wherein 1-3 methylene units of L are independently replaced by -Cy-, and 1-2 additional methylene units of L are optionally and independently replaced with —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is an optionally substituted bivalent, saturated or partially unsaturated, straight or branched C₁₋₃ hydrocarbon chain, wherein 2-3 methylene units of L are independently replaced by -Cy-, and 1-2 additional methylene units of L are optionally and independently replaced with —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is an optionally substituted bivalent, saturated or partially unsaturated, straight or branched C₁₋₃ hydrocarbon chain, wherein 0-2 methylene units of L are independently replaced with —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is an optionally substituted bivalent, saturated or partially unsaturated, straight or branched C₁₋₃ hydrocarbon chain, wherein 1 methylene unit of L is replaced by -Cy-, and 1-2 additional methylene units of L are optionally and independently replaced with —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is an optionally substituted bivalent, saturated or partially unsaturated, straight or branched C₁₋₃ hydrocarbon chain, wherein 2 methylene units of L are independently replaced by -Cy-, and 1 additional methylene unit of L is optionally and independently replaced with —CHF—, —CF₂—, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O. In some embodiments, L is an optionally substituted bivalent, saturated or partially unsaturated, straight or branched C₁₋₃ hydrocarbon chain, wherein 3 methylene units of L are independently replaced by -Cy-. In some embodiments, L is an optionally substituted bivalent, saturated or partially unsaturated, straight or branched C₁₋₃ hydrocarbon chain, wherein 1 methylene unit of L is replaced by —C(O)—, and 1-2 additional methylene units of L are optionally and independently replaced with -Cy-, —CHF—, —CF₂—, —O—, —NR—, —S—, —OC(O)—, —C(O)O—, —S(O)—, —S(O)₂—, —NRS(O)₂—, —S(O)₂NR—, —NRC(O)—, —C(O)NR—, —OC(O)NR—, or —NRC(O)O.

In some embodiments, L is:

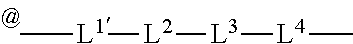

wherein
@ represents the point of attachment to Ring W;
L^{1′} is a covalent bond or an optionally substituted bivalent, saturated or partially unsaturated, straight or branched C₁₋₆ hydrocarbon chain, wherein 1-2 methylene units of L^{1′} are optionally and independently replaced by -Cy^{L1}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—;
L² is a covalent bond or an optionally substituted bivalent, saturated or partially unsaturated, straight or branched C₁₋₆ hydrocarbon chain, wherein 1-2 methylene units of L² are optionally and independently replaced by -Cy^{L2}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—;
L³ is a covalent bond or an optionally substituted bivalent, saturated or partially unsaturated, straight or branched C₁₋₆ hydrocarbon chain, wherein 1-2 methylene units of L³ are optionally and independently replaced by -Cy^{L3}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—;
L⁴ is a covalent bond or an optionally substituted bivalent, saturated or partially unsaturated, straight or branched C₁₋₆ hydrocarbon chain, wherein 1-2 methylene units of L⁴ are optionally and independently replaced by -Cy^{L4}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—; and
each of -Cy^{L1}-, Cy^{L2}-, -Cy^{L3}, and -Cy^{L4}- is independently -Cy-, wherein -Cy- is as defined above and described herein.

In some embodiments, L is @-L^{1′}-L²-L³-. In some embodiments, L is -L^{1′}-L². In some embodiments, L is ^{@}-L^{1′}-.

In some embodiments, L^{1′} is a covalent bond or an optionally substituted bivalent, saturated or partially unsaturated, straight or branched C₁₋₆ hydrocarbon chain, wherein 1 methylene unit of L^{1′} is optionally replaced by -Cy^{L1}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—.

In some embodiments, L^{1′} is a covalent bond. In some embodiments. L^{1′} is an optionally substituted bivalent, saturated or partially unsaturated, straight or branched C₁₋₆ hydrocarbon chain, wherein 1-2 methylene units of L^{1′} are optionally and independently replaced by -Cy^{L1}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—. In some embodiments. L^{1′} is -Cy^{L1}-. In some embodiments, L^{1′} is an optionally substituted bivalent, saturated or partially unsaturated, straight or branched C₁₋₆ hydrocarbon chain. In some embodiments. L^{1′} is an optionally substituted bivalent, saturated or partially unsaturated, straight or branched C₁₋₃ hydrocarbon chain. In some embodiments, L^{1′} is an optionally substituted bivalent, saturated or partially unsaturated, straight or branched C₁₋₆ hydrocarbon chain, wherein 1 methylene unit of L^{1′} is optionally and independently replaced by —O— or —NR—. In some embodiments, L^{1′} is —O—. In some embodiments, L^{1′} is optionally substituted —CH₂—, —CH₂CH₂—, —CH═CH—, —C≡C—, —CH₂CH₂CH₂—, or —CH═CHCH₂—, —CH₂CH═CH—, —C≡CCH₂—, or —CH₂C≡C—. In some embodiments, L^{1′} is —CH₂—, —CH₂CH₂—, —CH═CH—, —C≡C—, —CH₂CH₂CH₂—, or —CH═CHCH₂—, —CH₂CH═CH—, —C≡CCH₂—, or —CH₂Cl≡C—.

In some embodiments, L² is a covalent bond or an optionally substituted bivalent, saturated or partially unsaturated, straight or branched C₁₋₆ hydrocarbon chain, wherein 1 methylene unit of L^{1′} is optionally replaced by -Cy^{L1}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—.

In some embodiments, L² is a covalent bond. In some embodiments, L² is an optionally substituted bivalent, saturated or partially unsaturated, straight or branched C₁₋₆ hydrocarbon chain, wherein 1-2 methylene units of L² are optionally and independently replaced by -Cy^{L2}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—. In some embodiments, L² is -Cy^{L2}-. In some embodiments, L² is an optionally substituted bivalent, saturated or partially unsaturated, straight or branched C₁₋₆ hydrocarbon chain. In some embodiments, L² is an optionally substituted bivalent, saturated or partially unsaturated, straight or branched C₁₋₃ hydrocarbon chain. In some embodiments, L² is —O—. In some embodiments, L² is optionally substituted —CH₂—, —CH₂CH₂—, —CH═CH—, —C≡C—, —CH₂CH₂CH₂—, or —CH═CHCH₂—, —CH₂CH═CH—, —C≡CCH₂—, or —CH₂C≡C—. In some embodiments, L² is —CH₂—, —CH₂CH₂—, —CH═CH—, —C≡C—, —CH₂CH₂CH₂—, or —CH═CHCH₂—, —CH₂CH═CH—, —C≡CCH₂—, or —CH₂C≡C—.

In some embodiments, L³ is a covalent bond or an optionally substituted bivalent, saturated or partially unsaturated, straight or branched C₁₋₆ hydrocarbon chain, wherein 1 methylene unit of L^{1′} is optionally replaced by -Cy^{L1}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—.

In some embodiments. L³ is a covalent bond. In some embodiments. L³ is an optionally substituted bivalent, saturated or partially unsaturated, straight or branched C₁₋₆ hydrocarbon chain, wherein 1-2 methylene units of L³ are optionally and independently replaced by -Cy^{L3}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—. In some embodiments, L³ is -Cy^{L3}-. In some embodiments, L³ is an optionally substituted bivalent, saturated or partially unsaturated, straight or branched C₁₋₆ hydrocarbon chain. In some embodiments. L³ is an optionally substituted bivalent, saturated or partially unsaturated, straight or branched C₁₋₃ hydrocarbon chain. In some embodiments, L³ is —O—. In some embodiments, L³ is optionally substituted —CH₂—, —CH₂CH₂—, —CH═CH—, —C≡C—, —CH₂CH₂CH₂—, or —CH═CHCH₂—, —CH₂CH═CH—, —C≡CCH₂—, or —CH₂C≡C—. In some embodiments, L³ is —CH₂—, —CH₂CH₂—, —CH═CH—, —C≡C—, —CH₂CH₂CH₂—, or —CH═CHCH₂—, —CH₂CH═CH—, —C≡CCH₂—, or —CH₂C≡C—.

In some embodiments, L⁴ is a covalent bond or an optionally substituted bivalent, saturated or partially unsaturated, straight or branched C₁₋₆ hydrocarbon chain, wherein 1 methylene unit of L^{1′} is optionally replaced by -Cy^{L1}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—.

In some embodiments, L⁴ is a covalent bond. In some embodiments, L⁴ is an optionally substituted bivalent, saturated or partially unsaturated, straight or branched C₁₋₆ hydrocarbon chain, wherein 1-2 methylene units of L⁴ are optionally and independently replaced by -Cy^{L4}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—. In some embodiments, L⁴ is -Cy^{L4}-. In some embodiments, L⁴ is an optionally substituted bivalent, saturated or partially unsaturated, straight or branched C₁₋₆ hydrocarbon chain. In some embodiments, L⁴ is an optionally substituted bivalent, saturated or partially unsaturated, straight or branched C₁₋₃ hydrocarbon chain. In some embodiments, L⁴ is —O—. In some embodiments, L⁴ is optionally substituted —CH₂—, —CH₂CH₂—, —CH═CH—, —C≡C—, —CH₂CH₂CH₂—, or —CH═CHCH₂—, —CH₂CH═CH—, —C≡CCH₂—, or —CH₂C≡C—. In some embodiments, L⁴ is —CH₂—, —CH₂CH₂—, —CH═CH—, —C≡C—, —CH₂CH₂CH₂—, or —CH═CHCH₂—, —CH₂CH═CH—, —C≡CCH₂—, or —CH₂C≡C—.

As defined above and described herein, each of -Cy^{L1}-, Cy^{L2}-, -Cy^{L3}-, and -Cy^{L4}- is independently -Cy-, wherein -Cy- is as defined above and described herein.

In some embodiments, -Cy^{L1}- is an optionally substituted 8-10 membered bicyclic arylenyl. In some embodiments, -Cy^{L1}- is an optionally substituted 3-7 membered saturated or partially unsaturated carbocyclylenyl. In some embodiments, -Cy^{L1}- is an optionally substituted 6-11 membered saturated or partially unsaturated spiro carbocyclylenyl. In some embodiments, -Cy^{L1}- is an 8-10 membered bicyclic saturated or partially unsaturated carbocyclylenyl. In some embodiments, -Cy^{L1}- is an optionally substituted a 4-7 membered saturated or partially unsaturated heterocyclylenyl having 1-2 heteroatoms independently selected from nitrogen, oxygen, and sulfur. In some embodiments, -Cy^{L1}- is an optionally substituted 6-11 membered saturated or partially unsaturated spiro heterocyclylenyl having 1-3 heteroatoms independently selected from nitrogen, oxygen, and sulfur. In some embodiments, -Cy^{L1}- is an optionally substituted 8-10 membered bicyclic saturated or partially unsaturated heterocyclylenyl having 1-2 heteroatoms independently selected from nitrogen, oxygen, and sulfur. In some embodiments, -Cy^{L1}- is an optionally substituted 5-6 membered heteroarylenyl having 1-4 heteroatoms independently selected from nitrogen, oxygen. In some embodiments, -Cy^{L1}- is an optionally substituted 8-10 membered bicyclic heteroarylenyl having 1-5 heteroatoms independently selected from nitrogen, oxygen, and sulfur.

In some embodiments, each -Cy^{L1}- is independently a ring selected from phenylenyl, an 8-10 membered bicyclic arylenyl, a 4-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur.

In some embodiments, -Cy^{L1}- is an 8-10 membered bicyclic arylenyl. In some embodiments, -Cy^{L1}- is a 3-7 membered saturated or partially unsaturated carbocyclylenyl. In some embodiments, -Cy^{L1}- is a 6-11 membered saturated or partially unsaturated spiro carbocyclylenyl. In some embodiments, -Cy^{L1}- is an 8-10 membered bicyclic saturated or partially unsaturated carbocyclylenyl. In some embodiments, -Cy^{L1}- is a 4-7 membered saturated or partially unsaturated heterocyclylenyl having 1-2 heteroatoms independently selected from nitrogen, oxygen, and sulfur. In some embodiments, -Cy^{L1}- is a 6-11 membered saturated or partially unsaturated spiro heterocyclylenyl having 1-3 heteroatoms independently selected from nitrogen, oxygen, and sulfur. In some embodiments, -Cy^{L1}- is an 8-10 membered bicyclic saturated or partially unsaturated heterocyclylenyl having 1-2 heteroatoms independently selected from nitrogen, oxygen, and sulfur. In some embodiments, -Cy^{L1}- is a 5-6 membered heteroarylenyl having 1-4 heteroatoms independently selected from nitrogen, oxygen. In some embodiments, -Cy^{L1}- is an 8-10 membered bicyclic heteroarylenyl having 1-5 heteroatoms independently selected from nitrogen, oxygen, and sulfur.

In some embodiments, -Cy^{L1}- is an optionally substituted bivalent ring selected from phenylenyl, an 8-10 membered bicyclic arylenyl, a 3-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur.

In some embodiments, -Cy^{L1}- is an optionally substituted bivalent ring selected from phenylenyl, an 8-10 membered bicyclic arylenyl, a 3-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated or partially unsatured heterocyclylenyl having 1-2 heteroatoms independently selected from nitrogen, oxygen, and sulfur, and a 5-6 membered heteroarylenyl having 1-4 heteroatoms independently selected from nitrogen, oxygen, and sulfur.

In some embodiments, -Cy^{L1}- is an optionally substituted phenylenyl. In some embodiments, -Cy^{L1}- is phenylenyl, optionally substituted with halogen (e.g., fluoro or chloro), —CN, C₁₋₆ aliphatic optionally further substituted with halogen (e.g., fluoro or chloro), —S(O)₂R^{∘}, or —OR^{∘}, wherein R^{∘} is hydrogen or C₁₋₆ aliphatic. In some embodiments, -Cy^{L1}- is phenylenyl, optionally substituted with halogen (e.g., fluoro or chloro) or C₁₋₆ aliphatic.

In some embodiments, -Cy^{L1}- is an optionally substituted 5-6 membered saturated or partially unsaturated heterocyclylenyl having 1-2 heteroatoms independently selected from nitrogen, oxygen, and sulfur. In some embodiments, -Cy^{L1}- is an optionally substituted 5 membered saturated or partially unsaturated heterocyclylenyl having 1-2 heteroatoms independently selected from nitrogen, oxygen, and sulfur. In some embodiments, -Cy^{L1}- is an optionally substituted pyrrolidinylenyl. In some embodiments, -Cy^{L1}- is pyrrolidinylenyl, optionally substituted with halogen (e.g., fluoro or chloro), —CN, C₁₋₆ aliphatic optionally further substituted with halogen (e.g., fluoro or chloro), —S(O)₂R^{∘}, or —OR^{∘}, wherein R^{∘} is hydrogen or C₁₋₆ aliphatic. In some embodiments, -Cy^{L1}- is pyrrolidinylenyl, optionally substituted with halogen (e.g., fluoro or chloro) or C₁₋₆ aliphatic. In some embodiments, -Cy^{L1}- is an optionally substituted 6 membered saturated or partially unsaturated heterocyclylenyl having 1-2 heteroatoms independently selected from nitrogen, oxygen, and sulfur. In some embodiments, -Cy^{L1}- is an optionally substituted piperadinylenyl. In some embodiments, -Cy^{L1}- is piperadinylenyl, optionally substituted with halogen (e.g., fluoro or chloro), —CN, C₁₋₆ aliphatic optionally further substituted with halogen (e.g., fluoro or chloro), —S(O)₂R^{∘}, or —OR^{∘}, wherein R^{∘} is hydrogen or C₁₋₆ aliphatic. In some embodiments, -Cy^{L1}- is piperadinylenyl, optionally substituted with halogen (e.g., fluoro or chloro) or C₁₋₆ aliphatic. In some embodiments, -Cy^{L1}- is an optionally substituted piperazinylenyl. In some embodiments, -Cy^{L1}- is piperazinylenyl, optionally substituted with halogen (e.g., fluoro or chloro), —CN. C₁₋₆ aliphatic optionally further substituted with halogen (e.g., fluoro or chloro), —S(O)₂R^{∘}, or —OR^{∘}, wherein R^{∘} is hydrogen or C₁₋₆ aliphatic. In some embodiments, -Cy^{L1}- is piperazinylenyl, optionally substituted with halogen (e.g., fluoro or chloro) or C₁₋₆ aliphatic.

In some embodiments, -Cy^{L1}- is an optionally substituted 5 membered heteroarylenyl having 1-4 heteroatoms independently selected from nitrogen, oxygen. In some embodiments, -Cy^{L1}- is an optionally substituted pyrrolylenyl, pyrazolylenyl, imidazolylenyl, or triazolylenyl. In some embodiments, -Cy^{L1}- is pyrrolylenyl, pyrazolylenyl, imidazolylenyl, or triazolylenyl, optionally substituted with halogen (e.g., fluoro or chloro), —CN, C₁₋₆ aliphatic optionally further substituted with halogen (e.g., fluoro or chloro), —S(O)₂R^{∘}, or —OR^{∘}, wherein R^{∘} is hydrogen or C₁₋₆ aliphatic. In some embodiments, -Cy^{L1}- is pyrrolylenyl, pyrazolylenyl, imidazolylenyl, or triazolylenyl, optionally substituted with halogen (e.g., fluoro or chloro) or C₁₋₆ aliphatic. In some embodiments, -Cy^{L1}- is an optionally substituted pyrazolylenyl. In some embodiments, —C^{L1}— is pyrazolylenyl, optionally substituted with halogen (e.g., fluoro or chloro), —CN, C₁₋₆ aliphatic optionally further substituted with halogen (e.g., fluoro or chloro), —S(O)₂R^{∘}, or —OR^{∘}, wherein R^{∘} is hydrogen or C₁₋₆ aliphatic. In some embodiments, -Cy^{L1}- is pyrazolylenyl, optionally substituted with halogen (e.g., fluoro or chloro) or C₁₋₆ aliphatic. In some embodiments, -Cy^{L1}- is an optionally substituted imidazolylenyl. In some embodiments, -Cy^{L1}- is imidazolylenyl, optionally substituted with halogen (e.g., fluoro or chloro), —CN, C₁₋₆ aliphatic optionally further substituted with halogen (e.g., fluoro or chloro), or —OR^{∘}, wherein R^{∘} is hydrogen or C₁₋₆ aliphatic. In some embodiments, -Cy^{L1}- is imidazolylenyl, optionally substituted with halogen (e.g., fluoro or chloro) or C₁₋₆ aliphatic.

In some embodiments, -Cy^{L1}- is an optionally substituted 6 membered heteroarylenyl having 1-4 heteroatoms independently selected from nitrogen, oxygen. In some embodiments, -Cy^{L1}- is an optionally substituted pyridinylenyl, pyridazinylenyl, pyrimidinylenyl, pyrazinylenyl, or triazinylenyl. In some embodiments, -Cy^{L1}- is pyridinylenyl, pyridazinylenyl, pyrimidinylenyl, pyrazinylenyl, or triazinylenyl, optionally substituted with halogen (e.g., fluoro or chloro), —CN, C₁₋₆ aliphatic optionally further substituted with halogen (e.g., fluoro or chloro), —S(O)₂R^{∘}, or —OR^{∘}, wherein R^{∘} is hydrogen or C₁₋₆ aliphatic. In some embodiments, -Cy^{L1}- is pyridinylenyl, pyridazinylenyl, pyrimidinylenyl, pyrazinylenyl, or triazinylenyl, optionally substituted with halogen (e.g., fluoro or chloro) or C₁₋₆ aliphatic. In some embodiments, -Cy^{L1}- is an optionally substituted pyridinylenyl. In some embodiments, -Cy^{L1}- is pyridinylenyl, optionally substituted with halogen (e.g., fluoro or chloro), —CN, C₁₋₆ aliphatic optionally further substituted with halogen (e.g., fluoro or chloro), —S(O)₂R^{∘}, or —OR^{∘}, wherein R^{∘} is hydrogen or C₁₋₆ aliphatic. In some embodiments, -Cy^{L1}- is pyridinylenyl, optionally substituted with halogen (e.g., fluoro or chloro) or C₁₋₆ aliphatic. In some embodiments, -Cy^{L1}- is an optionally substituted pyridazinylenyl. In some embodiments, -Cy^{L1}- is pyridazinylenyl, optionally substituted with halogen (e.g., fluoro or chloro), —CN, C₁₋₆ aliphatic optionally further substituted with halogen (e.g., fluoro or chloro), —S(O)₂R^{∘}, or —OR^{∘}, wherein R^{∘} is hydrogen or C₁₋₆ aliphatic. In some embodiments, -Cy^{L1}- is pyridazinylenyl, optionally substituted with halogen (e.g., fluoro or chloro) or C₁₋₆ aliphatic. In some embodiments, -Cy^{L1}- is an optionally substituted pyridinonylenyl, pyridazinonylenyl, pyrimidinonylenyl, pyrazinonylenyl, or triazinonylenyl. In some embodiments, -Cy^{L1}- is pyridinonylenyl, pyridazinonylenyl, pyrimidinonylenyl, pyrazinonylenyl, or triazinonylenyl, optionally substituted with halogen (e.g., fluoro or chloro), —CN, C₁₋₆ aliphatic optionally further substituted with halogen (e.g., fluoro or chloro), —S(O)₂R^{∘}, or —OR^{∘}, wherein R^{∘} is hydrogen or C₁₋₆ aliphatic. In some embodiments, -Cy^{L1}- is pyridinonylenyl, pyridazinonylenyl, pyrimidinonylenyl, pyrazinonylenyl, or triazinonylenyl, optionally substituted with halogen (e.g., fluoro or chloro) or C₁₋₆ aliphatic. In some embodiments, -Cy^{L1}- is an optionally substituted pyridinonylenyl. In some embodiments. -Cy^{L1}- is pyridinonylenyl, optionally substituted with halogen (e.g., fluoro or chloro), —CN, C₁₋₆ aliphatic optionally further substituted with halogen (e.g., fluoro or chloro), —S(O)₂R^{∘}, or —OR^{∘}, wherein R^{∘} is hydrogen or C₁₋₆ aliphatic. In some embodiments, -Cy^{L1}- is pyridinonylenyl, optionally substituted with halogen (e.g., fluoro or chloro) or C₁₋₆ aliphatic.

In some embodiments, -Cy^{L1}- is an optionally substituted cyclopropyl. In some embodiments, -Cy^{L1}- is an optionally substituted 5 membered saturated or partially unsaturated carbocyclylenyl. In some embodiments, -Cy^{L1}- is an optionally substituted cyclopentanylenyl or cyclopentenylenyl. In some embodiments, -Cy^{L1}- is an optionally substituted 6 membered saturated or partially unsaturated carbocyclylenyl. In some embodiments, -Cy^{L1}- is an optionally substituted cyclohexanylenyl or cyclohexenylenyl. In some embodiments, -Cy^{L1}- is an optionally substituted naphthalenylenyl.

In some embodiments, -Cy^{L1}- is an optionally substituted 9 membered bicyclic heteroarylenyl having 1-5 heteroatoms independently selected from nitrogen, oxygen, and sulfur. In some embodiments, -Cy^{L1}- is an optionally substituted indolylenyl, azaindolylenyl, isoindolylenyl, azaisoindolylenyl, indazolylenyl, azaindazolylenyl, benzimidazolylenyl, or azabenzimidazolylenyl. In some embodiments, —Cy^{L1}- is an optionally substituted indolylenyl. In some embodiments, -Cy^{L1}- is an optionally substituted benzothiophenylenyl, benzofuranylenyl, isobenzofuranylenyl, benzoisooxazolylenyl, benzoisothiazolylenyl, benzoxazolylenyl, benzothiazolylenyl, or benzothiadiazolylenyl.

In some embodiments, -Cy^{L1}- is an optionally substituted 8-11 membered saturated or partially unsaturated spiro heterocyclylenyl having 1-3 heteroatoms independently selected from nitrogen, oxygen, and sulfur. In some embodiments. -Cy^{L1}- is an optionally substituted 9-11 membered saturated or partially unsaturated spiro heterocyclylenyl having 1-3 heteroatoms independently selected from nitrogen, oxygen, and sulfur. In some embodiments, -Cy^{L1}- is an optionally substituted 9 membered saturated or partially unsaturated spiro heterocyclylenyl having 1-3 heteroatoms independently selected from nitrogen, oxygen, and sulfur. In some embodiments, -Cy^{L1}- is an optionally substituted 9 membered saturated or partially unsaturated spiro heterocyclylenyl having 1-3 nitrogen heteroatoms. In some embodiments, -Cy^{L1}- is an optionally substituted 4,6-spiro heterocyclylenyl having 1-3 heteroatoms independently selected from nitrogen, oxygen, and sulfur. In some embodiments, -Cy^{L1}- is an optionally substituted

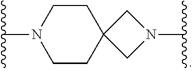 [SMILES: *N1CCC2(CC1)CN(*)C2]

In some embodiments, -Cy^{L1}- is an optionally substituted 10 membered saturated or partially unsaturated spiro heterocyclylenyl having 1-3 heteroatoms independently selected from nitrogen, oxygen, and sulfur. In some embodiments, -Cy^{L1}- is an optionally substituted 10 membered saturated or partially unsaturated spiro heterocyclylenyl having 1-3 nitrogen heteroatoms. In some embodiments, -Cy^{L1}- is an optionally substituted 5,6-spiro heterocyclylenyl having 1-3 heteroatoms independently selected from nitrogen, oxygen, and sulfur.

In some embodiments, -Cy^{L1}- is an optionally substituted 11 membered saturated or partially unsaturated spiro heterocyclylenyl having 1-3 heteroatoms independently selected from nitrogen, oxygen, and sulfur. In some embodiments, -Cy^{L1}- is an optionally substituted 11 membered saturated or partially unsaturated spiro heterocyclylenyl having 1-3 nitrogen heteroatoms. In some embodiments, -Cy^{L1}- is an optionally substituted 6,6-spiro heterocyclylenyl having 1-3 heteroatoms independently selected from nitrogen, oxygen, and sulfur.

In some embodiments, -Cy^{L1}- is optionally substituted

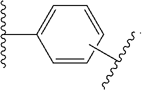

In some embodiments, -Cy^{L1}- is

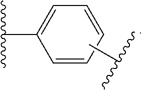

In some embodiments, -Cy^{L1}- is

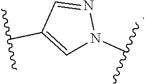 [SMILES: *c1cnn(*)c1]

In some embodiments, -Cy^{L1}- is

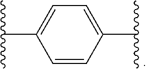 [SMILES: *c1ccc(*)cc1]

In some embodiments, -Cy^{L1}- is

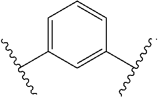 [SMILES: *c1cccc(*)c1]

In some embodiments, -Cy^{L1}- is

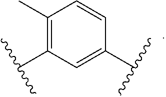 [SMILES: *c1ccc(C)c(*)c1]

In some embodiments, -Cy^{L1}- is

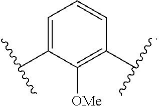 [SMILES: *c1cccc(*)c1OC]

In some embodiments, -Cy^{L1}- is

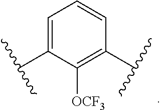 [SMILES: *c1cccc(*)c1OC(F)(F)F]

In some embodiments, -Cy^{L1}- is

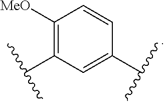 [SMILES: *c1ccc(OC)c(*)c1]

In some embodiments, -Cy^{L1}- is

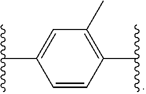 [SMILES: *c1ccc(*)c(C)c1]

In some embodiments, -Cy^{L1}- is

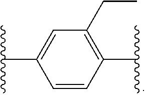 [SMILES: *c1ccc(*)c(CC)c1]

In some embodiments, -Cy^{L1}- is

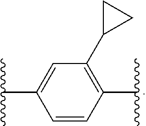 [SMILES: *c1ccc(*)c(C2CC2)c1]

In some embodiments, -Cy^{L1}- is

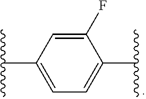 [SMILES: *c1ccc(*)c(F)c1]

In some embodiments, -Cy^{L1}- is

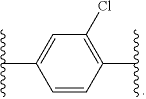 [SMILES: *c1ccc(*)c(Cl)c1]

In some embodiments, -Cy^{L1}- is

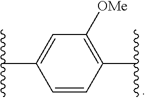 [SMILES: *c1ccc(*)c(OC)c1]

In some embodiments, -Cy^{L1}- is

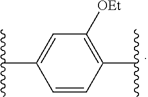 [SMILES: *c1ccc(*)c(OCC)c1]

In some embodiments, -Cy^{L1}- is

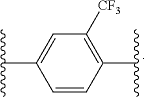 [SMILES: *c1ccc(*)c(C(F)(F)F)c1]

In some embodiments, -Cy^{L1}- is

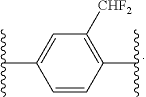 [SMILES: *c1ccc(*)c(C(F)F)c1]

In some embodiments, -Cy^{L1}- is

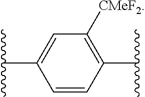

In some embodiments, -Cy^{L1}- is

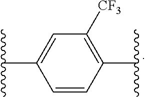 [SMILES: *c1ccc(*)c(C(F)(F)F)c1]

In some embodiments, -Cy^{L1}- is

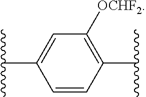 [SMILES: *c1ccc(*)c(OC(F)F)c1]

In some embodiments, -Cy^{L1}- is

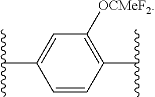

In some embodiments, -Cy^{L1}- is

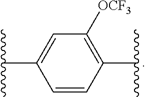 [SMILES: *c1ccc(*)c(OC(F)(F)F)c1]

In some embodiments, -Cy^{L1}- is

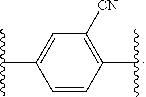 [SMILES: *c1ccc(*)c(C#N)c1]

In some embodiments, -Cy^{L1}- is

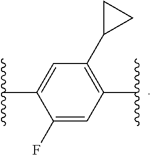 [SMILES: *c1cc(C2CC2)c(*)cc1F]

In some embodiments, -Cy^{L1}- is

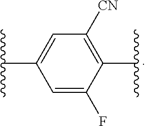 [SMILES: *c1cc(F)c(*)c(C#N)c1]

In some embodiments, -Cy^{L1}- is

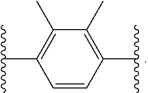 [SMILES: *c1ccc(*)c(C)c1C]

In some embodiments, -Cy^{L1}- is

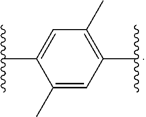 [SMILES: *c1cc(C)c(*)cc1C]

In some embodiments, -Cy^{L1}- is

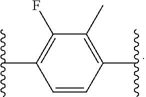 [SMILES: *c1ccc(*)c(F)c1C]

In some embodiments, -Cy^{L1}- is

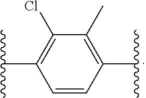 [SMILES: *c1ccc(*)c(Cl)c1C]

In some embodiments, -Cy^{L1}- is

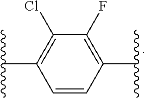 [SMILES: *c1ccc(*)c(Cl)c1F]

In some embodiments, -Cy^{L1}- is

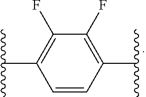 [SMILES: *c1ccc(*)c(F)c1F]

In some embodiments, -Cy^{L1}- is

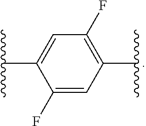 [SMILES: *c1cc(F)c(*)cc1F]

In some embodiments, -Cy^{L1}- is

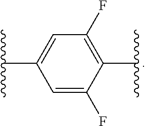 [SMILES: *c1cc(F)c(*)c(F)c1]

In some embodiments, -Cy^{L1}- is

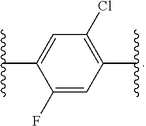 [SMILES: *c1cc(Cl)c(*)cc1F]

In some embodiments, -Cy^{L1}- is

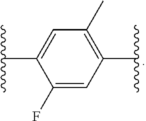 [SMILES: *c1cc(F)c(*)cc1C]

In some embodiments, -Cy^{L1}- is

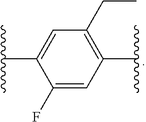 [SMILES: *c1cc(CC)c(*)cc1F]

In some embodiments, -Cy^{L1}- is

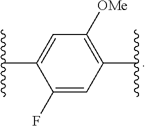 [SMILES: *c1cc(OC)c(*)cc1F]

In some embodiments, -Cy^{L1}- is

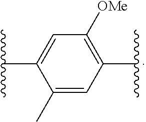 [SMILES: *c1cc(OC)c(*)cc1C]

In some embodiments, -Cy^{L1}- is

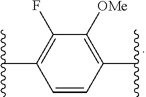 [SMILES: *c1ccc(*)c(OC)c1F]

In some embodiments, -Cy^{L1}- is

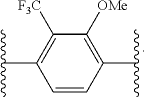 [SMILES: *c1ccc(*)c(C(F)(F)F)c1OC]

In some embodiments, -Cy^{L1}- is

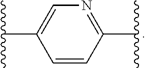 [SMILES: *c1ccc(*)nc1]

In some embodiments, -Cy^{L1}- is

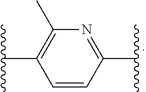 [SMILES: *c1ccc(*)c(C)n1]

In some embodiments, -Cy^{L1}- is

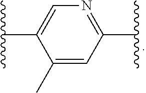 [SMILES: *c1cc(C)c(*)cn1]

In some embodiments, -Cy^{L1}- is

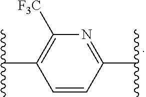 [SMILES: *c1ccc(*)c(C(F)(F)F)n1]

In some embodiments, -Cy^{L1}- is

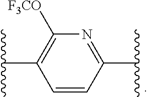 [SMILES: *c1ccc(*)c(OC(F)(F)F)n1]

In some embodiments, -Cy^{L1}- is

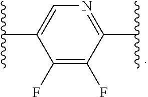 [SMILES: *c1cnc(*)c(F)c1F]

In some embodiments, -Cy^{L1}- is

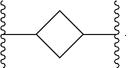 [SMILES: *C1CC(*)C1]

In some embodiments, -Cy^{L1}- is

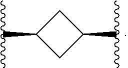 [SMILES: *[C@H]1C[C@@H](*)C1]

In some embodiments -Cy^{L1}- is

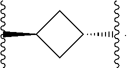 [SMILES: *[C@H]1C[C@H](*)C1]

In some embodiments, -Cy^{L1}- is

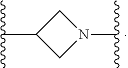 [SMILES: *C1CN(*)C1]

In some embodiments, -Cy^{L1}- is

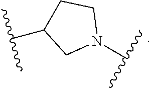 [SMILES: *C1CCN(*)C1]

In some embodiments, -Cy^{L1}- is

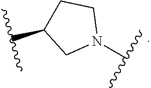 [SMILES: *[C@H]1CCN(*)C1]

In some embodiments, -Cy^{L1}- is

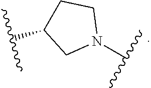 [SMILES: *[C@@H]1CCN(*)C1]

In some embodiments, -Cy^{L1}- is

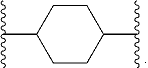 [SMILES: *C1CCC(*)CC1]

In some embodiments, -Cy^{L1}- is

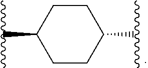 [SMILES: *[C@H]1CC[C@H](*)CC1]

In some embodiments, -Cy^{L1}- is

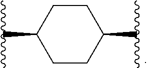 [SMILES: *[C@H]1CC[C@@H](*)CC1]

In some embodiments, -Cy^{L1}- is

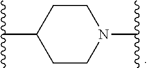 [SMILES: *C1CCN(*)CC1]

In some embodiments, -Cy^{L1}- is

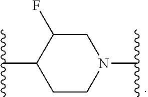 [SMILES: *C1CCN(*)CC1F]

In some embodiments, -Cy^{L1}- is

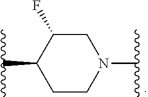 [SMILES: *[C@@H]1CCN(*)C[C@H]1F]

In some embodiments, -Cy^{L1}- is

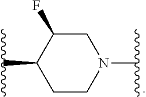 [SMILES: *[C@@H]1CCN(*)C[C@@H]1F]

In some embodiments, -Cy^{L1}- is

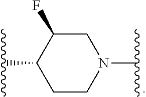 [SMILES: *[C@H]1CCN(*)C[C@@H]1F]

In some embodiments, -Cy^{L1}- is

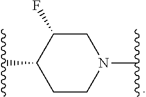 [SMILES: *[C@H]1CCN(*)C[C@H]1F]

In some embodiments, -Cy^{L1}- is

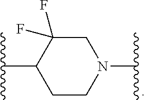 [SMILES: *C1CCN(*)CC1(F)F]

In some embodiments, -Cy^{L1}- is

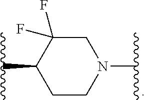 [SMILES: *[C@@H]1CCN(*)CC1(F)F]

In some embodiments, -Cy^{L1}- is

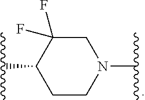 [SMILES: *[C@H]1CCN(*)CC1(F)F]

In some embodiments, -Cy^{L1}- is

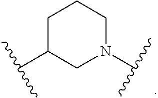 [SMILES: *C1CCCN(*)C1]

In some embodiments, -Cy^{L1}- is

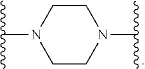 [SMILES: *N1CCN(*)CC1]

In some embodiments, -Cy^{L1}- is

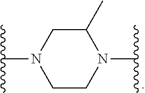 [SMILES: *N1CCN(*)C(C)C1]

In some embodiments, -Cy^{L1}- is

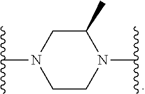 [SMILES: *N1CCN(*)[C@H](C)C1]

In some embodiments, -Cy^{L1}- is

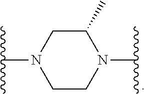 [SMILES: *N1CCN(*)[C@@H](C)C1]

In some embodiments, -Cy^{L1}- is

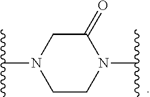 [SMILES: *N1CCN(*)C(=O)C1]

In some embodiments, -Cy^{L1}- is

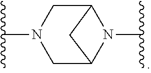 [SMILES: *N1CC2CC(C1)N2*]

In some embodiments, -Cy^{L1}- is

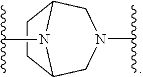 [SMILES: *N1CC2CCC(C1)N2*]

In some embodiments, -Cy^{L1}- is

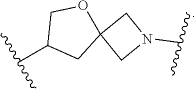 [SMILES: *C1COC2(C1)CN(*)C2]

In some embodiments, -Cy^{L1}- is

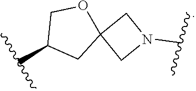 [SMILES: *[C@H]1COC2(C1)CN(*)C2]

In some embodiments, -Cy^{L1}- is

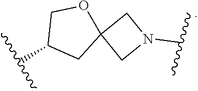 [SMILES: *[C@@H]1COC2(C1)CN(*)C2]

In some embodiments, -Cy^{L1}- is

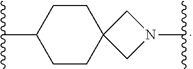 [SMILES: *C1CCC2(CC1)CN(*)C2]

In some embodiments, -Cy^{L1}- is

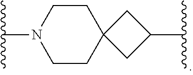 [SMILES: *C1CC2(CCN(*)CC2)C1]

In some embodiments, -Cy^{L1}- is

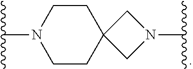 [SMILES: *N1CCC2(CC1)CN(*)C2]

In some embodiments, -Cy^{L1}- is

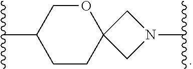 [SMILES: *C1CCC2(CN(*)C2)OC1]

In some embodiments, -Cy^{L1}- is

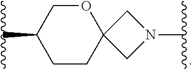 [SMILES: *[C@@H]1CCC2(CN(*)C2)OC1]

In some embodiments, -Cy^{L1}- is

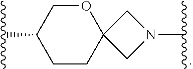 [SMILES: *[C@H]1CCC2(CN(*)C2)OC1]

In some embodiments, -Cy^{L1}- is

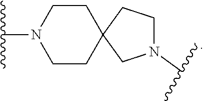 [SMILES: *N1CCC2(CC1)CCN(*)C2]

In some embodiments, -Cy^{L1}- is

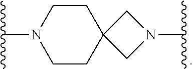 [SMILES: *N1CCC2(CC1)CN(*)C2]

In some embodiments, -Cy^{L1}- is

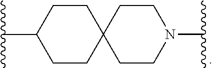 [SMILES: *C1CCC2(CC1)CCN(*)CC2]

In some embodiments, -Cy^{L1}- is

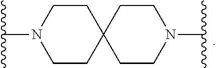 [SMILES: *N1CCC2(CC1)CCN(*)CC2]

In some embodiments, —Cy^{L1}- is

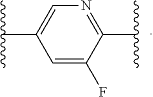 [SMILES: *c1cnc(*)c(F)c1]

In some embodiments, -Cy^{L2}- is an optionally substituted 8-10 membered bicyclic arylenyl. In some embodiments, -Cy^{L2}- is an optionally substituted 3-7 membered saturated or partially unsaturated carbocyclylenyl. In some embodiments, -Cy^{L2}- is an optionally substituted 6-11 membered saturated or partially unsaturated spiro carbocyclylenyl. In some embodiments, -Cy^{L2}- is an 8-10 membered bicyclic saturated or partially unsaturated carbocyclylenyl. In some embodiments, -Cy^{L2}- is an optionally substituted a 4-7 membered saturated or partially unsaturated heterocyclylenyl having 1-2 heteroatoms independently selected from nitrogen, oxygen, and sulfur. In some embodiments, -Cy^{L1}- is an optionally substituted 6-11 membered saturated or partially unsaturated spiro heterocyclylenyl having 1-3 heteroatoms independently selected from nitrogen, oxygen, and sulfur. In some embodiments, -Cy^{L1}- is an optionally substituted 8-10 membered bicyclic saturated or partially unsaturated heterocyclylenyl having 1-2 heteroatoms independently selected from nitrogen, oxygen, and sulfur. In some embodiments, -Cy^{L1}- is an optionally substituted 5-6 membered heteroarylenyl having 1-4 heteroatoms independently selected from nitrogen, oxygen. In some embodiments, -Cy^{L2}- is an optionally substituted 8-10 membered bicyclic heteroarylenyl having 1-5 heteroatoms independently selected from nitrogen, oxygen, and sulfur.

In some embodiments, each -Cy^{L1}- is independently a ring selected from phenylenyl, an 8-10 membered bicyclic arylenyl, a 4-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur.

In some embodiments, -Cy^{L1}- is an 8-10 membered bicyclic arylenyl. In some embodiments, -Cy^{L2}- is a 3-7 membered saturated or partially unsaturated carbocyclylenyl. In some embodiments, -Cy^{L2}- is a 6-11 membered saturated or partially unsaturated spiro carbocyclylenyl. In some embodiments, -Cy^{L2}- is an 8-10 membered bicyclic saturated or partially unsaturated carbocyclylenyl. In some embodiments, -Cy^{L2}- is a 4-7 membered saturated or partially unsaturated heterocyclylenyl having 1-2 heteroatoms independently selected from nitrogen, oxygen, and sulfur. In some embodiments, -Cy^{L1}- is a 6-11 membered saturated or partially unsaturated spiro heterocyclylenyl having 1-3 heteroatoms independently selected from nitrogen, oxygen, and sulfur. In some embodiments, -Cy^{L2}- is an 8-10 membered bicyclic saturated or partially unsaturated heterocyclylenyl having 1-2 heteroatoms independently selected from nitrogen, oxygen, and sulfur. In some embodiments, -Cy^{L1}- is a 5-6 membered heteroarylenyl having 1-4 heteroatoms independently selected from nitrogen, oxygen. In some embodiments, -Cy^{L2}- is an 8-10 membered bicyclic heteroarylenyl having 1-5 heteroatoms independently selected from nitrogen, oxygen, and sulfur.

In some embodiments, -Cy^{L2}- is an optionally substituted bivalent ring selected from phenylenyl, an 8-10 membered bicyclic arylenyl, a 3-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur.

In some embodiments, -Cy^{L2}- is an optionally substituted bivalent ring selected from phenylenyl, an 8-10 membered bicyclic arylenyl, a 3-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated or partially unsatured heterocyclylenyl having 1-2 heteroatoms independently selected from nitrogen, oxygen, and sulfur, and a 5-6 membered heteroarylenyl having 1-4 heteroatoms independently selected from nitrogen, oxygen, and sulfur.

In some embodiments, -Cy^{L2}- is an optionally substituted phenylenyl. In some embodiments, -Cy^{L2} is phenylenyl, optionally substituted with halogen (e.g., fluoro or chloro), —CN, C₁₋₆ aliphatic optionally further substituted with halogen (e.g., fluoro or chloro), —S(O)₂R^{∘}, or —OR^{∘}, wherein R^{∘} is hydrogen or C₁₋₆ aliphatic. In some embodiments, -Cy^{L2}- is phenylenyl, optionally substituted with halogen (e.g., fluoro or chloro) or C₁₋₆ aliphatic.

In some embodiments, -Cy^{L2}- is an optionally substituted 5-6 membered saturated or partially unsaturated heterocyclylenyl having 1-2 heteroatoms independently selected from nitrogen, oxygen, and sulfur. In some embodiments, -Cy^{L2}- is an optionally substituted 5 membered saturated or partially unsaturated heterocyclylenyl having 1-2 heteroatoms independently selected from nitrogen, oxygen, and sulfur. In some embodiments, -Cy^{L2}- is an optionally substituted pyrrolidinylenyl. In some embodiments, -Cy^{L2}- is pyrrolidinylenyl, optionally substituted with halogen (e.g., fluoro or chloro), —CN, C₁₋₆ aliphatic optionally further substituted with halogen (e.g., fluoro or chloro), —S(O)₂R^{∘}, or —OR^{∘}, wherein R^{∘} is hydrogen or C₁₋₆ aliphatic. In some embodiments, -Cy^{L2}- is pyrrolidinylenyl, optionally substituted with halogen (e.g., fluoro or chloro) or C₁₋₆ aliphatic. In some embodiments, -Cy^{L2}- is an optionally substituted 6 membered saturated or partially unsaturated heterocyclylenyl having 1-2 heteroatoms independently selected from nitrogen, oxygen, and sulfur. In some embodiments, -Cy^{L2}- is an optionally substituted piperadinylenyl. In some embodiments, -Cy^{L2}- is piperadinylenyl, optionally substituted with halogen (e.g., fluoro or chloro), —CN, C₁₋₆ aliphatic optionally further substituted with halogen (e.g., fluoro or chloro), —S(O)₂R^{∘}, or —OR^{∘}, wherein R^{∘} is hydrogen or C₁₋₆ aliphatic. In some embodiments, -Cy^{L2}- is piperadinylenyl, optionally substituted with halogen (e.g., fluoro or chloro) or C₁₋₆ aliphatic. In some embodiments, -Cy^{L2}- is an optionally substituted piperazinylenyl. In some embodiments, -Cy^{L2} is piperazinylenyl, optionally substituted with halogen (e.g., fluoro or chloro), —CN, C₁₋₆ aliphatic optionally further substituted with halogen (e.g., fluoro or chloro), —S(O)₂R^{∘}, or —OR^{∘}, wherein R^{∘} is hydrogen or C₁₋₆ aliphatic. In some embodiments, -Cy^{L2}- is piperazinylenyl, optionally substituted with halogen (e.g., fluoro or chloro) or C₁₋₆ aliphatic.

In some embodiments, -Cy^{L2}- is an optionally substituted 5 membered heteroarylenyl having 1-4 heteroatoms independently selected from nitrogen, oxygen. In some embodiments, -Cy^{L2} is an optionally substituted pyrrolylenyl, pyrazolylenyl, imidazolylenyl, or triazolylenyl. In some embodiments, -Cy^{L2} is pyrrolylenyl, pyrazolylenyl, imidazolylenyl, or triazolylenyl, optionally substituted with halogen (e.g., fluoro or chloro), —CN, C₁₋₆ aliphatic optionally further substituted with halogen (e.g., fluoro or chloro), —S(O)₂R^{∘}, or —OR^{∘}, wherein R^{∘} is hydrogen or C₁₋₆ aliphatic. In some embodiments, -Cy^{L1}- is pyrrolylenyl, pyrazolylenyl, imidazolylenyl, or triazolylenyl, optionally substituted with halogen (e.g., fluoro or chloro) or C₁₋₆ aliphatic. In some embodiments, -Cy^{L2}- is an optionally substituted pyrazolylenyl. In some embodiments, -Cy^{L2}- is pyrazolylenyl, optionally substituted with halogen (e.g., fluoro or chloro), —CN, C₁₋₆ aliphatic optionally further substituted with halogen (e.g., fluoro or chloro), —S(O)₂R^{∘}, or —OR^{∘}, wherein R^{∘} is hydrogen or C₁₋₆ aliphatic. In some embodiments, -Cy^{L2}- is pyrazolylenyl, optionally substituted with halogen (e.g., fluoro or chloro) or C₁₋₆ aliphatic. In some embodiments, -Cy^{L2}- is an optionally substituted imidazolylenyl. In some embodiments, -Cy^{L2}- is imidazolylenyl, optionally substituted with halogen (e.g., fluoro or chloro), —CN, C₁₋₆ aliphatic optionally further substituted with halogen (e.g., fluoro or chloro), or —OR^{∘}, wherein R^{∘} is hydrogen or C₁₋₆ aliphatic. In some embodiments, -Cy^{L2} is imidazolylenyl, optionally substituted with halogen (e.g., fluoro or chloro) or C₁₋₆ aliphatic.

In some embodiments, -Cy^{L2}- is an optionally substituted 6 membered heteroarylenyl having 1-4 heteroatoms independently selected from nitrogen, oxygen. In some embodiments, -Cy^{L1}- is an optionally substituted pyridinylenyl, pyridazinylenyl, pyrimidinylenyl, pyrazinylenyl, or triazinylenyl. In some embodiments, -Cy^{L2}- is pyridinylenyl, pyridazinylenyl, pyrimidinylenyl, pyrazinylenyl, or triazinylenyl, optionally substituted with halogen (e.g., fluoro or chloro), —CN, C₁₋₆ aliphatic optionally further substituted with halogen (e.g., fluoro or chloro), —S(O)₂R^{∘}, or —OR^{∘}, wherein R^{∘} is hydrogen or C₁₋₆ aliphatic. In some embodiments, -Cy^{L1}- is pyridinylenyl, pyridazinylenyl, pyrimidinylenyl, pyrazinylenyl, or triazinylenyl, optionally substituted with halogen (e.g., fluoro or chloro) or C₁₋₆ aliphatic. In some embodiments, -Cy^{L2}- is an optionally substituted pyridinylenyl. In some embodiments, -Cy^{L2} is pyridinylenyl, optionally substituted with halogen (e.g., fluoro or chloro), —CN, C₁₋₆ aliphatic optionally further substituted with halogen (e.g., fluoro or chloro), —S(O)₂R^{∘}, or —OR^{∘}, wherein R^{∘} is hydrogen or C₁₋₆ aliphatic. In some embodiments, -Cy^{L2}- is pyridinylenyl, optionally substituted with halogen (e.g., fluoro or chloro) or C₆ aliphatic. In some embodiments, -Cy^{L1}- is an optionally substituted pyridazinylenyl. In some embodiments, -Cy² is pyridazinylenyl, optionally substituted with halogen (e.g., fluoro or chloro), —CN, C₁₋₆ aliphatic optionally further substituted with halogen (e.g., fluoro or chloro), —S(O)₂R^{∘}, or —OR^{∘}, wherein R^{∘} is hydrogen or C₁₋₆ aliphatic. In some embodiments, -Cy^{L2}- is pyridazinylenyl, optionally substituted with halogen (e.g., fluoro or chloro) or C₁₋₆ aliphatic. In some embodiments, -Cy^{L1}- is an optionally substituted pyridinonylenyl, pyridazinonylenyl, pyrimidinonylenyl, pyrazinonylenyl, or triazinonylenyl. In some embodiments, -Cy^{L1}- is pyridinonylenyl, pyridazinonylenyl, pyrimidinonylenyl, pyrazinonylenyl, or triazinonylenyl, optionally substituted with halogen (e.g., fluoro or chloro), —CN, C₁₋₆ aliphatic optionally further substituted with halogen (e.g., fluoro or chloro), —S(O)₂R^{∘}, or —OR^{∘}, wherein R^{∘} is hydrogen or C₁₋₆ aliphatic. In some embodiments, -Cy^{L1}- is pyridinonylenyl, pyridazinonylenyl, pyrimidinonylenyl, pyrazinonylenyl, or triazinonylenyl, optionally substituted with halogen (e.g., fluoro or chloro) or C₁₋₆ aliphatic. In some embodiments, -Cy^{L2}- is an optionally substituted pyridinonylenyl. In some embodiments, -Cy^{L2}- is pyridinonylenyl, optionally substituted with halogen (e.g., fluoro or chloro), —CN, C₁₋₆ aliphatic optionally further substituted with halogen (e.g., fluoro or chloro), —S(O)₂R^{∘}, or —OR^{∘}, wherein R^{∘} is hydrogen or C₁₋₆ aliphatic. In some embodiments, -Cy^{L2}- is pyridinonylenyl, optionally substituted with halogen (e.g., fluoro or chloro) or C₁₋₆ aliphatic.

In some embodiments, -Cy^{L1}- is an optionally substituted cyclopropyl. In some embodiments, -Cy^{L2} is an optionally substituted 5 membered saturated or partially unsaturated carbocyclylenyl. In some embodiments, -Cy^{L1}- is an optionally substituted cyclopentanylenyl or cyclopentenylenyl. In some embodiments, -Cy^{L2}- is an optionally substituted 6 membered saturated or partially unsaturated carbocyclylenyl. In some embodiments, -Cy^{L1}- is an optionally substituted cyclohexanylenyl or cyclohexenylenyl. In some embodiments, -Cy^{L1}- is an optionally substituted naphthalenylenyl.

In some embodiments, -Cy^{L2}- is an optionally substituted 9 membered bicyclic heteroarylenyl having 1-5 heteroatoms independently selected from nitrogen, oxygen, and sulfur. In some embodiments, -Cy^{L2} is an optionally substituted indolylenyl, azaindolylenyl, isoindolylenyl, azaisoindolylenyl, indazolylenyl, azaindazolylenyl, benzimidazolylenyl, or azabenzimidazolylenyl. In some embodiments, -Cy^{L2}- is an optionally substituted indolylenyl. In some embodiments, -Cy^{L1}- is an optionally substituted benzothiophenylenyl, benzofuranylenyl, isobenzofuranylenyl, benzoisooxazolylenyl, benzoisothiazolylenyl, benzoxazolylenyl, benzothiazolylenyl, or benzothiadiazolylenyl.

In some embodiments, -Cy^{L2}- is an optionally substituted 8-11 membered saturated or partially unsaturated spiro heterocyclylenyl having 1-3 heteroatoms independently selected from nitrogen, oxygen, and sulfur. In some embodiments, -Cy^{L2}- is an optionally substituted 9-11 membered saturated or partially unsaturated spiro heterocyclylenyl having 1-3 heteroatoms independently selected from nitrogen, oxygen, and sulfur. In some embodiments, -Cy^{L2}- is an optionally substituted 9 membered saturated or partially unsaturated spiro heterocyclylenyl having 1-3 heteroatoms independently selected from nitrogen, oxygen, and sulfur. In some embodiments, -Cy^{L1}- is an optionally substituted 9 membered saturated or partially unsaturated spiro heterocyclylenyl having 1-3 nitrogen heteroatoms. In some embodiments, -Cy^{L1}- is an optionally substituted 4,6-spiro heterocyclylenyl having 1-3 heteroatoms independently selected from nitrogen, oxygen, and sulfur. In some embodiments, -Cy^{L2}- is an optionally substituted

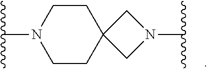 [SMILES: *N1CCC2(CC1)CN(*)C2]

In some embodiments, -Cy^{L1}- is an optionally substituted 10 membered saturated or partially unsaturated spiro heterocyclylenyl having 1-3 heteroatoms independently selected from nitrogen, oxygen, and sulfur. In some embodiments, -Cy^{L1}- is an optionally substituted 10 membered saturated or partially unsaturated spiro heterocyclylenyl having 1-3 nitrogen heteroatoms. In some embodiments, -Cy^{L2}- is an optionally substituted 5,6-spiro heterocyclylenyl having 1-3 heteroatoms independently selected from nitrogen, oxygen, and sulfur.

In some embodiments, -Cy^{L1}- is an optionally substituted 11 membered saturated or partially unsaturated spiro heterocyclylenyl having 1-3 heteroatoms independently selected from nitrogen, oxygen, and sulfur. In some embodiments, -Cy^{L2}- is an optionally substituted 11 membered saturated or partially unsaturated spiro heterocyclylenyl having 1-3 nitrogen heteroatoms. In some embodiments, -Cy^{L2}- is an optionally substituted 6,6-spiro heterocyclylenyl having 1-3 heteroatoms independently selected from nitrogen, oxygen, and sulfur.

In some embodiments, -Cy^{L2}- is optionally substituted

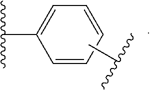

In some embodiments, -Cy^{L2}- is

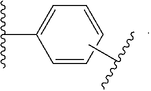

In some embodiments, -Cy^{L2}- is

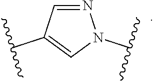 [SMILES: *c1cnn(*)c1]

In some embodiments, -Cy^{L2}- is

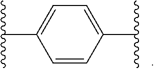 [SMILES: *c1ccc(*)cc1]

In some embodiments, -Cy^{L2}- is

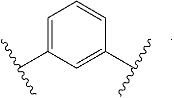 [SMILES: *c1cccc(*)c1]

In some embodiments, -Cy^{L2}- is

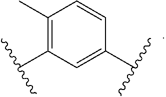 [SMILES: *c1ccc(C)c(*)c1]

In some embodiments, -Cy^{L2}- is

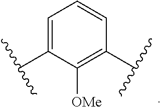 [SMILES: *c1cccc(*)c1OC]

In some embodiments, -Cy^{L2}- is

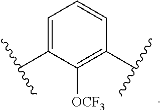 [SMILES: *c1cccc(*)c1OC(F)(F)F]

In some embodiments, -Cy^{L2}- is

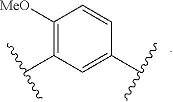 [SMILES: *c1ccc(OC)c(*)c1]

In some embodiments, -Cy^{L2}- is

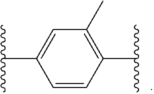 [SMILES: *c1ccc(*)c(C)c1]

In some embodiments, -Cy^{L2}- is

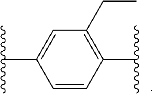 [SMILES: *c1ccc(*)c(CC)c1]

In some embodiments, -Cy^{L2}- is

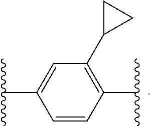 [SMILES: *c1ccc(*)c(C2CC2)c1]

In some embodiments, -Cy^{L2}- is

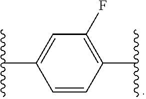 [SMILES: *c1ccc(*)c(F)c1]

In some embodiments, -Cy^{L2}- is

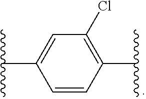 [SMILES: *c1ccc(*)c(Cl)c1]

In some embodiments, -Cy^{L2}- is

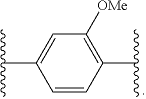 [SMILES: *c1ccc(*)c(OC)c1]

In some embodiments, -Cy^{L2}- is

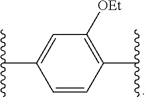 [SMILES: *c1ccc(*)c(OCC)c1]

In some embodiments, -Cy^{L2}- is

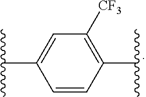 [SMILES: *c1ccc(*)c(C(F)(F)F)c1]

In some embodiments, -Cy^{L2}- is

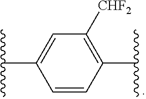 [SMILES: *c1ccc(*)c(C(F)F)c1]

In some embodiments, -Cy^{L2}- is

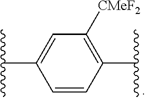

In some embodiments, -Cy^{L2}- is

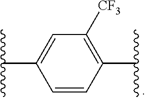 [SMILES: *c1ccc(*)c(C(F)(F)F)c1]

In some embodiments, -Cy^{L2}- is

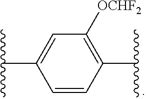 [SMILES: *c1ccc(*)c(OC(F)F)c1]

In some embodiments, -Cy^{L2}- is

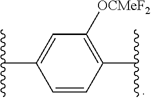

In some embodiments, -Cy^{L2}- is

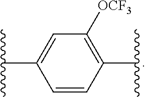 [SMILES: *c1ccc(*)c(OC(F)(F)F)c1]

In some embodiments, -Cy^{L2}- is

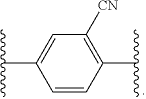 [SMILES: *c1ccc(*)c(C#N)c1]

In some embodiments, -Cy^{L2}- is

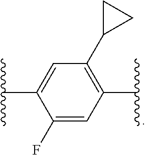 [SMILES: *c1cc(C2CC2)c(*)cc1F]

In some embodiments, -Cy^{L2} is

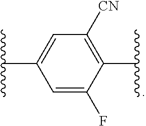 [SMILES: *c1cc(F)c(*)c(C#N)c1]

In some embodiments, -Cy^{L2}- is

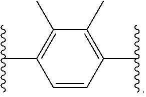 [SMILES: *c1ccc(*)c(C)c1C]

In some embodiments, -Cy^{L2}- is

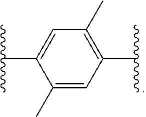 [SMILES: *c1cc(C)c(*)cc1C ; "."]

In some embodiments, -Cy^{L2} is

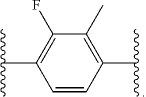 [SMILES: *c1ccc(*)c(F)c1C ; "."]

In some embodiments, -Cy^{L2}- is

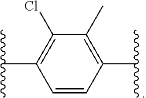 [SMILES: *c1ccc(*)c(Cl)c1C ; "."]

In some embodiments, -Cy^{L2}- is

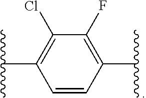 [SMILES: *c1ccc(*)c(Cl)c1F ; "."]

In some embodiments, -Cy^{L2} is

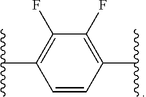 [SMILES: *c1ccc(*)c(F)c1F ; "."]

In some embodiments, -Cy^{L2} is

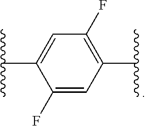 [SMILES: *c1cc(F)c(*)cc1F ; "."]

In some embodiments, -Cy^{L2} is

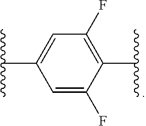 [SMILES: *c1cc(F)c(*)c(F)c1 ; "."]

In some embodiments, -Cy^{L2}- is

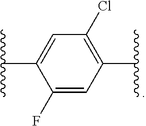 [SMILES: *c1cc(Cl)c(*)cc1F ; "."]

In some embodiments, -Cy^{L2}- is

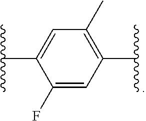 [SMILES: *c1cc(F)c(*)cc1C ; "."]

In some embodiments, -Cy^{L2}- is

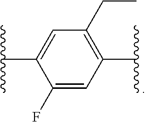 [SMILES: *c1cc(CC)c(*)cc1F ; "."]

In some embodiments, -Cy^{L2}- is

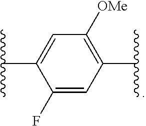 [SMILES: *c1cc(OC)c(*)cc1F ; "."]

In some embodiments, -Cy^{L2}- is

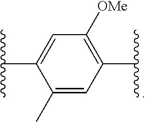 [SMILES: *c1cc(OC)c(*)cc1C ; "."]

In some embodiments, -Cy^{L2}- is

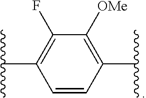 [SMILES: *c1ccc(*)c(OC)c1F ; "."]

In some embodiments, -Cy^{L2}- is

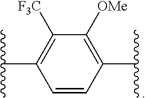 [SMILES: *c1ccc(*)c(C(F)(F)F)c1OC ; "."]

In some embodiments, -Cy^{L2}- is

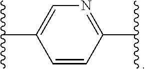 [SMILES: *c1ccc(*)nc1 ; "."]

In some embodiments, Cy^{L2}- is

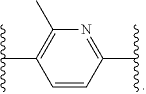 [SMILES: *c1ccc(*)c(C)n1]

In some embodiments, -Cy^{L2}- is

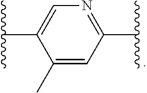 [SMILES: *c1cc(C)c(*)cn1]

In some embodiments, -Cy^{L2}- is

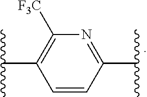 [SMILES: *c1ccc(*)c(C(F)(F)F)n1]

In some embodiments, -Cy^{L2}- is

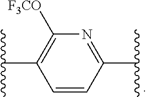 [SMILES: *c1ccc(*)c(OC(F)(F)F)n1]

In some embodiments, -Cy^{L2}- is

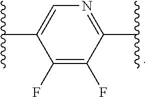 [SMILES: *c1cnc(*)c(F)c1F]

In some embodiments, -Cy^{L2}- is

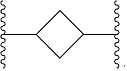 [SMILES: *C1CC(*)C1]

In some embodiments, -Cy^{L2}- is

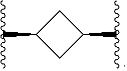 [SMILES: *[C@H]1C[C@@H](*)C1]

In some embodiments, -Cy^{L2}- is

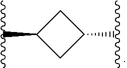 [SMILES: *[C@H]1C[C@H](*)C1]

In some embodiments, -Cy^{L2}- is

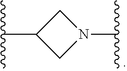 [SMILES: *C1CN(*)C1]

In some embodiments, -Cy^{L2}- is

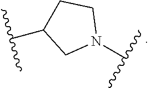 [SMILES: *C1CCN(*)C1]

In some embodiments, -Cy^{L2}- is

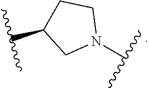 [SMILES: *[C@H]1CCN(*)C1]

In some embodiments, -Cy^{L2}- is

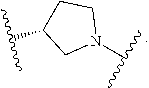 [SMILES: *[C@@H]1CCN(*)C1]

In some embodiments, -Cy^{L2}- is

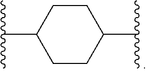 [SMILES: *C1CCC(*)CC1]

In some embodiments, -Cy^{L2}- is

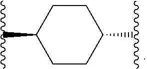 [SMILES: *[C@H]1CC[C@H](*)CC1]

In some embodiments, -Cy^{L2}- is

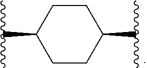 [SMILES: *[C@H]1CC[C@@H](*)CC1]

In some embodiments, -Cy^{L2} is

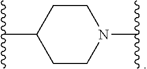 [SMILES: *C1CCN(*)CC1 ; "."]

In some embodiments, -Cy^{L2}- is

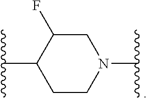 [SMILES: *C1CCN(*)CC1F ; "."]

In some embodiments, -Cy^{L2}- is

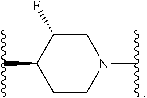 [SMILES: *[C@@H]1CCN(*)C[C@H]1F ; "."]

In some embodiments, -Cy^{L2}- is

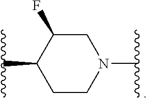 [SMILES: *[C@@H]1CCN(*)C[C@@H]1F ; "."]

In some embodiments, -Cy^{L2}- is

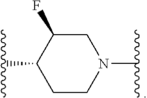 [SMILES: *[C@H]1CCN(*)C[C@@H]1F ; "."]

In some embodiments, -Cy^{L2}- is

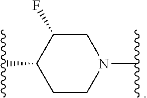 [SMILES: *[C@H]1CCN(*)C[C@H]1F ; "."]

In some embodiments, -Cy^{L2} is

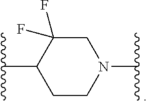 [SMILES: *C1CCN(*)CC1(F)F ; "."]

In some embodiments, -Cy^{L2}- is

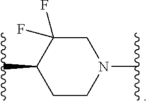 [SMILES: *[C@@H]1CCN(*)CC1(F)F ; "."]

In some embodiments, -Cy^{L2}- is

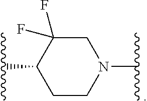 [SMILES: *[C@H]1CCN(*)CC1(F)F ; "."]

In some embodiments, Cy^{L2}- is

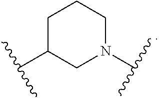 [SMILES: *C1CCCN(*)C1 ; "."]

In some embodiments, -Cy^{L2}- is

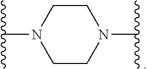 [SMILES: *N1CCN(*)CC1 ; "."]

In some embodiments, -Cy^{L2}- is

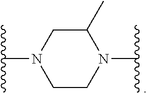 [SMILES: *N1CCN(*)C(C)C1 ; "."]

In some embodiments, -Cy^{L2}- is

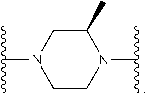 [SMILES: *N1CCN(*)[C@H](C)C1 ; "."]

In some embodiments, -Cy^{L2}- is

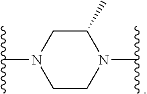 [SMILES: *N1CCN(*)[C@@H](C)C1 ; "."]

In some embodiments, -Cy^{L2}- is

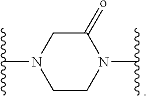

In some embodiments, -Cy^{L2}- is

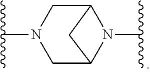 [SMILES: *N1CC2CC(C1)N2*]

In some embodiments, -Cy^{L2}- is

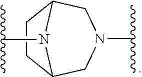 [SMILES: *N1CC2CCC(C1)N2*]

In some embodiments, -Cy^{L2}- is

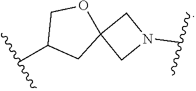 [SMILES: *C1COC2(C1)CN(*)C2]

In some embodiments, -Cy^{L2}- is

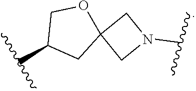 [SMILES: *[C@H]1COC2(C1)CN(*)C2]

In some embodiments, -Cy^{L2}- is

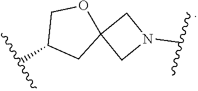 [SMILES: *[C@@H]1COC2(C1)CN(*)C2]

In some embodiments, -Cy^{L2} is

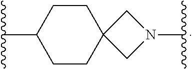 [SMILES: *C1CCC2(CC1)CN(*)C2]

In some embodiments, -Cy^{L2}- is

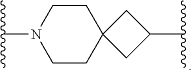 [SMILES: *C1CC2(CCN(*)CC2)C1]

In some embodiments, -Cy^{L2}- is

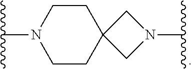 [SMILES: *N1CCC2(CC1)CN(*)C2]

In some embodiments, -Cy^{L2}- is

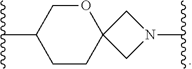 [SMILES: *C1CCC2(CN(*)C2)OC1]

In some embodiments, -Cy^{L2}- is

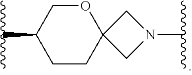 [SMILES: *[C@@H]1CCC2(CN(*)C2)OC1]

In some embodiments, -Cy^{L2}- is

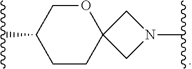 [SMILES: *[C@H]1CCC2(CN(*)C2)OC1]

In some embodiments, -Cy^{L2}- is

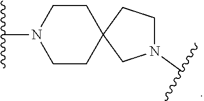 [SMILES: *N1CCC2(CC1)CCN(*)C2]

In some embodiments, -Cy^{L2}- is

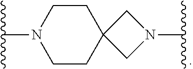 [SMILES: *N1CCC2(CC1)CN(*)C2]

In some embodiments, -Cy^{L2}- is

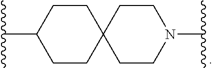 [SMILES: *C1CCC2(CC1)CCN(*)CC2]

In some embodiments, -Cy^{L2}- is

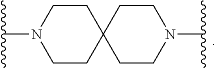 [SMILES: *N1CCC2(CC1)CCN(*)CC2]

In some embodiments, —Cy^{L2}- is

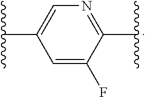 [SMILES: *c1cnc(*)c(F)c1]

In some embodiments, -Cy^{L3}- is an optionally substituted 8-10 membered bicyclic arylenyl. In some embodiments, -Cy^{L3}- is an optionally substituted 3-7 membered saturated or partially unsaturated carbocyclylenyl. In some embodiments, -Cy^{L3}- is an optionally substituted 6-11 membered saturated or partially unsaturated spiro carbocyclylenyl. In some embodiments, -Cy^{L3}- is an 8-10 membered bicyclic saturated or partially unsaturated carbocyclylenyl. In some embodiments, -Cy^{L3}- is an optionally substituted a 4-7 membered saturated or partially unsaturated heterocyclylenyl having 1-2 heteroatoms independently selected from nitrogen, oxygen, and sulfur. In some embodiments, -Cy^{L3}- is an optionally substituted 6-11 membered saturated or partially unsaturated spiro heterocyclylenyl having 1-3 heteroatoms independently selected from nitrogen, oxygen, and sulfur. In some embodiments, -Cy^{L3}- is an optionally substituted 8-10 membered bicyclic saturated or partially unsaturated heterocyclylenyl having 1-2 heteroatoms independently selected from nitrogen, oxygen, and sulfur. In some embodiments, -Cy^{L3}- is an optionally substituted 5-6 membered heteroarylenyl having 1-4 heteroatoms independently selected from nitrogen, oxygen. In some embodiments, -Cy^{L1}- is an optionally substituted 8-10 membered bicyclic heteroarylenyl having 1-5 heteroatoms independently selected from nitrogen, oxygen, and sulfur.

In some embodiments, each -Cy^{L3}- is independently a ring selected from phenylenyl, an 8-10 membered bicyclic arylenyl, a 4-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur.

In some embodiments, -Cy^{L3}- is an 8-10 membered bicyclic arylenyl. In some embodiments, -Cy^{L3}- is a 3-7 membered saturated or partially unsaturated carbocyclylenyl. In some embodiments, -Cy^{L3}- is a 6-11 membered saturated or partially unsaturated spiro carbocyclylenyl. In some embodiments, -Cy^{L3}- is an 8-10 membered bicyclic saturated or partially unsaturated carbocyclylenyl. In some embodiments, -Cy^{L3}- is a 4-7 membered saturated or partially unsaturated heterocyclylenyl having 1-2 heteroatoms independently selected from nitrogen, oxygen, and sulfur. In some embodiments, -Cy^{L1}- is a 6-11 membered saturated or partially unsaturated spiro heterocyclylenyl having 1-3 heteroatoms independently selected from nitrogen, oxygen, and sulfur. In some embodiments. -Cy^{L3}- is an 8-10 membered bicyclic saturated or partially unsaturated heterocyclylenyl having 1-2 heteroatoms independently selected from nitrogen, oxygen, and sulfur. In some embodiments, -Cy^{L3}- is a 5-6 membered heteroarylenyl having 1-4 heteroatoms independently selected from nitrogen, oxygen. In some embodiments, -Cy^{L3}- is an 8-10 membered bicyclic heteroarylenyl having 1-5 heteroatoms independently selected from nitrogen, oxygen, and sulfur.

In some embodiments, -Cy^{L3}- is an optionally substituted bivalent ring selected from phenylenyl, an 8-10 membered bicyclic arylenyl, a 3-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur.

In some embodiments, -Cy^{L3}- is an optionally substituted bivalent ring selected from phenylenyl, an 8-10 membered bicyclic arylenyl, a 3-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated or partially unsatured heterocyclylenyl having 1-2 heteroatoms independently selected from nitrogen, oxygen, and sulfur, and a 5-6 membered heteroarylenyl having 1-4 heteroatoms independently selected from nitrogen, oxygen, and sulfur.

In some embodiments, -Cy^{L3}- is an optionally substituted phenylenyl. In some embodiments, -Cy^{L3}- is phenylenyl, optionally substituted with halogen (e.g., fluoro or chloro), —CN, C₁₋₆ aliphatic optionally further substituted with halogen (e.g., fluoro or chloro), —S(O)₂R^{∘}, or —OR^{∘}, wherein R^{∘} is hydrogen or C₁₋₆ aliphatic. In some embodiments, -Cy^{L3}- is phenylenyl, optionally substituted with halogen (e.g., fluoro or chloro) or C₁₋₆ aliphatic.

In some embodiments, -Cy^{L3}- is an optionally substituted 5-6 membered saturated or partially unsaturated heterocyclylenyl having 1-2 heteroatoms independently selected from nitrogen, oxygen, and sulfur. In some embodiments, -Cy^{L3}- is an optionally substituted 5 membered saturated or partially unsaturated heterocyclylenyl having 1-2 heteroatoms independently selected from nitrogen, oxygen, and sulfur. In some embodiments, -Cy^{L3}- is an optionally substituted pyrrolidinylenyl. In some embodiments, -Cy^{L3}- is pyrrolidinylenyl, optionally substituted with halogen (e.g., fluoro or chloro), —CN, C₁₋₆ aliphatic optionally further substituted with halogen (e.g., fluoro or chloro), —S(O)₂R^{∘}, or —OR^{∘}, wherein R^{∘} is hydrogen or C₁₋₆ aliphatic. In some embodiments, -Cy^{L1}- is pyrrolidinylenyl, optionally substituted with halogen (e.g., fluoro or chloro) or C₁₋₆ aliphatic. In some embodiments, -Cy^{L3} is an optionally substituted 6 membered saturated or partially unsaturated heterocyclylenyl having 1-2 heteroatoms independently selected from nitrogen, oxygen, and sulfur. In some embodiments, -Cy^{L2}- is an optionally substituted piperadinylenyl. In some embodiments, -Cy^{L3}- is piperadinylenyl, optionally substituted with halogen (e.g., fluoro or chloro), —CN, C₁₋₆ aliphatic optionally further substituted with halogen (e.g., fluoro or chloro), —S(O)₂R^{∘}, or —OR^{∘}, wherein R^{∘} is hydrogen or C₁₋₆ aliphatic. In some embodiments, -Cy^{L3}- is piperadinylenyl, optionally substituted with halogen (e.g., fluoro or chloro) or C₁₋₆ aliphatic. In some embodiments, -Cy^{L3}- is an optionally substituted piperazinylenyl. In some embodiments, -Cy^{L3}- is piperazinylenyl, optionally substituted with halogen (e.g., fluoro or chloro), —CN, C₁₋₆ aliphatic optionally further substituted with halogen (e.g., fluoro or chloro), —S(O)₂R^{∘}, or —OR^{∘}, wherein R^{∘} is hydrogen or C₁₋₆ aliphatic. In some embodiments, -Cy^{L3}- is piperazinylenyl, optionally substituted with halogen (e.g., fluoro or chloro) or C—₆ aliphatic.

In some embodiments, -Cy^{L3}- is an optionally substituted 5 membered heteroarylenyl having 1-4 heteroatoms independently selected from nitrogen, oxygen. In some embodiments, -Cy^{L3}- is an optionally substituted pyrrolylenyl, pyrazolylenyl, imidazolylenyl, or triazolylenyl. In some embodiments, -Cy^{L1}- is pyrrolylenyl, pyrazolylenyl, imidazolylenyl, or triazolylenyl, optionally substituted with halogen (e.g., fluoro or chloro), —CN, C₁₋₆ aliphatic optionally further substituted with halogen (e.g., fluoro or chloro), —S(O)₂R^{∘}, or —OR^{∘}, wherein R^{∘} is hydrogen or C₁₋₆ aliphatic. In some embodiments, -Cy^{L1}- is pyrrolylenyl, pyrazolylenyl, imidazolylenyl, or triazolylenyl, optionally substituted with halogen (e.g., fluoro or chloro) or C₁₋₆ aliphatic. In some embodiments, -Cy^{L3}- is an optionally substituted pyrazolylenyl. In some embodiments, -Cy^{L3}- is pyrazolylenyl, optionally substituted with halogen (e.g., fluoro or chloro), —CN, C₁₋₆ aliphatic optionally further substituted with halogen (e.g., fluoro or chloro), —S(O)₂R^{∘}, or —OR^{∘}, wherein R^{∘} is hydrogen or C₆ aliphatic. In some embodiments, -Cy^{L3}- is pyrazolylenyl, optionally substituted with halogen (e.g., fluoro or chloro) or C₁₋₆ aliphatic. In some embodiments, -Cy^{L3}- is an optionally substituted imidazolylenyl. In some embodiments, -Cy^{L3}- is imidazolylenyl, optionally substituted with halogen (e.g., fluoro or chloro), —CN, C₁₋₆ aliphatic optionally further substituted with halogen (e.g., fluoro or chloro), or —OR^{∘}, wherein R^{∘} is hydrogen or C₁₋₆ aliphatic. In some embodiments, -Cy³ is imidazolylenyl, optionally substituted with halogen (e.g., fluoro or chloro) or C₁₋₆ aliphatic.

In some embodiments, -Cy^{L3}- is an optionally substituted 6 membered heteroarylenyl having 1-4 heteroatoms independently selected from nitrogen, oxygen. In some embodiments, -Cy^{L3}- is an optionally substituted pyridinylenyl, pyridazinylenyl, pyrimidinylenyl, pyrazinylenyl, or triazinylenyl. In some embodiments, -Cy^{L1}- is pyridinylenyl, pyridazinylenyl, pyrimidinylenyl, pyrazinylenyl, or triazinylenyl, optionally substituted with halogen (e.g., fluoro or chloro), —CN, C₁₋₆ aliphatic optionally further substituted with halogen (e.g., fluoro or chloro), —S(O)₂R^{∘}, or —OR^{∘}, wherein R^{∘} is hydrogen or C₁₋₆ aliphatic. In some embodiments, -Cy^{L3}- is pyridinylenyl, pyridazinylenyl, pyrimidinylenyl, pyrazinylenyl, or triazinylenyl, optionally substituted with halogen (e.g., fluoro or chloro) or C₁₋₆ aliphatic. In some embodiments, -Cy^{L3}- is an optionally substituted pyridinylenyl. In some embodiments, -Cy^{L3}- is pyridinylenyl, optionally substituted with halogen (e.g., fluoro or chloro), —CN, C₁₋₆ aliphatic optionally further substituted with halogen (e.g., fluoro or chloro), —S(O)₂R^{∘} or —OR^{∘}, wherein R^{∘} is hydrogen or C₁₋₆ aliphatic. In some embodiments, -Cy^{L3}- is pyridinylenyl, optionally substituted with halogen (e.g., fluoro or chloro) or C₁₋₆ aliphatic. In some embodiments, -Cy^{L3}- is an optionally substituted pyridazinylenyl. In some embodiments, -Cy^{L3}- is pyridazinylenyl, optionally substituted with halogen (e.g., fluoro or chloro), —CN, C₁₋₆ aliphatic optionally further substituted with halogen (e.g., fluoro or chloro), —S(O)₂R^{∘}, or —OR^{∘}, wherein R^{∘} is hydrogen or C₁₋₆ aliphatic. In some embodiments, -Cy^{L3}- is pyridazinylenyl, optionally substituted with halogen (e.g., fluoro or chloro) or C₁₋₆ aliphatic. In some embodiments, -Cy^{L3}- is an optionally substituted pyridinonylenyl, pyridazinonylenyl, pyrimidinonylenyl, pyrazinonylenyl, or triazinonylenyl. In some embodiments, -Cy^{L3}- is pyridinonylenyl, pyridazinonylenyl, pyrimidinonylenyl, pyrazinonylenyl, or triazinonylenyl, optionally substituted with halogen (e.g., fluoro or chloro), —CN, C₁₋₆ aliphatic optionally further substituted with halogen (e.g., fluoro or chloro), —S(O)₂R^{∘}, or —OR^{∘}, wherein R^{∘} is hydrogen or C₁₋₆ aliphatic. In some embodiments, -Cy^{L3}- is pyridinonylenyl, pyridazinonylenyl, pyrimidinonylenyl, pyrazinonylenyl, or triazinonylenyl, optionally substituted with halogen (e.g., fluoro or chloro) or C₁₋₆ aliphatic. In some embodiments, -Cy^{L3}- is an optionally substituted pyridinonylenyl. In some embodiments, -Cy^{L3}- is pyridinonylenyl, optionally substituted with halogen (e.g., fluoro or chloro), —CN, C₁₋₆ aliphatic optionally further substituted with halogen (e.g., fluoro or chloro), —S(O)₂R^{∘}, or —OR^{∘}, wherein R^{∘} is hydrogen or C₁₋₆ aliphatic. In some embodiments, -Cy^{L3}- is pyridinonylenyl, optionally substituted with halogen (e.g., fluoro or chloro) or C₁₋₆ aliphatic.

In some embodiments, -Cy^{L3}- is an optionally substituted cyclopropyl. In some embodiments, -Cy^{L3}- is an optionally substituted 5 membered saturated or partially unsaturated carbocyclylenyl. In some embodiments, -Cy^{L1}- is an optionally substituted cyclopentanylenyl or cyclopentenylenyl. In some embodiments, -Cy^{L3}- is an optionally substituted 6 membered saturated or partially unsaturated carbocyclylenyl. In some embodiments, -Cy^{L1}- is an optionally substituted cyclohexanylenyl or cyclohexenylenyl. In some embodiments, -Cy^{L3}- is an optionally substituted naphthalenylenyl.

In some embodiments, -Cy^{L3}- is an optionally substituted 9 membered bicyclic heteroarylenyl having 1-5 heteroatoms independently selected from nitrogen, oxygen, and sulfur. In some embodiments, -Cy^{L3}- is an optionally substituted indolylenyl, azaindolylenyl, isoindolylenyl, azaisoindolylenyl, indazolylenyl, azaindazolylenyl, benzimidazolylenyl, or azabenzimidazolylenyl. In some embodiments, -Cy^{L3}- is is an optionally substituted indolylenyl. In some embodiments, -Cy^{L3}- is an optionally substituted benzothiophenylenyl, benzofuranylenyl, isobenzofuranylenyl, benzoisooxazolylenyl, benzoisothiazolylenyl, benzoxazolylenyl, benzothiazolylenyl, or benzothiadiazolylenyl.

In some embodiments, -Cy^{L3}- is an optionally substituted 8-11 membered saturated or partially unsaturated spiro heterocyclylenyl having 1-3 heteroatoms independently selected from nitrogen, oxygen, and sulfur. In some embodiments, -Cy^{L3}- is an optionally substituted 9-11 membered saturated or partially unsaturated spiro heterocyclylenyl having 1-3 heteroatoms independently selected from nitrogen, oxygen, and sulfur. In some embodiments, -Cy^{L3}- is an optionally substituted 9 membered saturated or partially unsaturated spiro heterocyclylenyl having 1-3 heteroatoms independently selected from nitrogen, oxygen, and sulfur. In some embodiments, -Cy^{L3}- is an optionally substituted 9 membered saturated or partially unsaturated spiro heterocyclylenyl having 1-3 nitrogen heteroatoms. In some embodiments, -Cy^{L3}- is an optionally substituted 4,6-spiro heterocyclylenyl having 1-3 heteroatoms independently selected from nitrogen, oxygen, and sulfur. In some embodiments, -Cy^{L3} is an optionally substituted

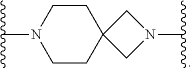 [SMILES: *N1CCC2(CC1)CN(*)C2]

In some embodiments, -Cy^{L3}- is an optionally substituted 10 membered saturated or partially unsaturated spiro heterocyclylenyl having 1-3 heteroatoms independently selected from nitrogen, oxygen, and sulfur. In some embodiments, -Cy^{L3}- is an optionally substituted 10 membered saturated or partially unsaturated spiro heterocyclylenyl having 1-3 nitrogen heteroatoms. In some embodiments, -Cy^{L1}- is an optionally substituted 5,6-spiro heterocyclylenyl having 1-3 heteroatoms independently selected from nitrogen, oxygen, and sulfur.

In some embodiments, -Cy^{L3}- is an optionally substituted 11 membered saturated or partially unsaturated spiro heterocyclylenyl having 1-3 heteroatoms independently selected from nitrogen, oxygen, and sulfur. In some embodiments, -Cy^{L3}- is an optionally substituted 11 membered saturated or partially unsaturated spiro heterocyclylenyl having 1-3 nitrogen heteroatoms. In some embodiments, -Cy^{L1}- is an optionally substituted 6,6-spiro heterocyclylenyl having 1-3 heteroatoms independently selected from nitrogen, oxygen, and sulfur.

In some embodiments, -Cy^{L3}- is optionally substituted

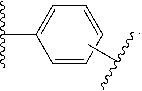

In some embodiments, -Cy^{L3}- is

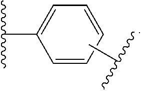

In some embodiments, -Cy^{L3}- is

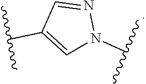 [SMILES: *c1cnn(*)c1]

In some embodiments, -Cy^{L3}- is

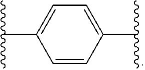 [SMILES: *c1ccc(*)cc1]

In some embodiments, -Cy^{L3}- is

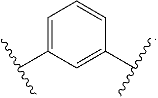 [SMILES: *c1cccc(*)c1]

In some embodiments, -Cy^{L3}- is

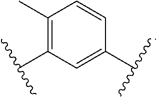 [SMILES: *c1ccc(C)c(*)c1]

In some embodiments, -Cy^{L3}- is

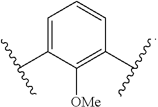 [SMILES: *c1cccc(*)c1OC]

In some embodiments, -Cy^{L3}- is

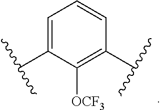 [SMILES: *c1cccc(*)c1OC(F)(F)F]

In some embodiments, -Cy^{L3}- is

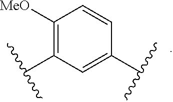 [SMILES: *c1ccc(OC)c(*)c1]

In some embodiments, -Cy^{L3}- is

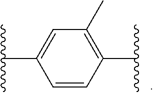 [SMILES: *c1ccc(*)c(C)c1]

In some embodiments, -Cy^{L3}- is

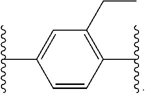 [SMILES: *c1ccc(*)c(CC)c1]

In some embodiments, -Cy^{L3}- is

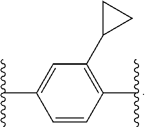 [SMILES: *c1ccc(*)c(C2CC2)c1]

In some embodiments, -Cy^{L3}- is

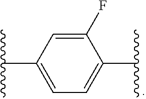 [SMILES: *c1ccc(*)c(F)c1]

In some embodiments, -Cy^{L3}- is

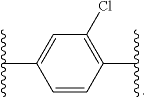 [SMILES: *c1ccc(*)c(Cl)c1]

In some embodiments, -Cy^{L3}- is

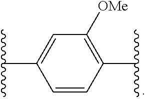 [SMILES: *c1ccc(*)c(OC)c1]

In some embodiments, -Cy^{L3}- is

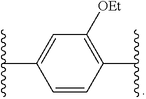 [SMILES: *c1ccc(*)c(OCC)c1]

In some embodiments, -Cy^{L3}- is

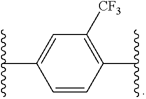 [SMILES: *c1ccc(*)c(C(F)(F)F)c1]

In some embodiments, -Cy^{L3}- is

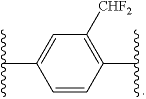 [SMILES: *c1ccc(*)c(C(F)F)c1]

In some embodiments, -Cy^{L3}- is

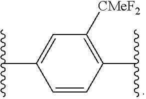

In some embodiments, -Cy^{L3}- is

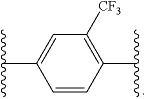 [SMILES: *c1ccc(*)c(C(F)(F)F)c1]

In some embodiments, -Cy^{L3}- is

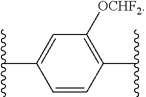 [SMILES: *c1ccc(*)c(OC(F)F)c1]

In some embodiments, -Cy^{L3}- is

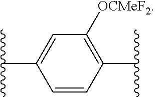

In some embodiments, -Cy^{L3}- is

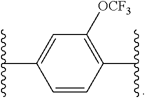 [SMILES: *c1ccc(*)c(OC(F)(F)F)c1]

In some embodiments, -Cy^{L3}- is

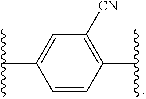 [SMILES: *c1ccc(*)c(C#N)c1]

In some embodiments, -Cy^{L3}- is

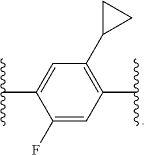 [SMILES: *c1cc(C2CC2)c(*)cc1F]

In some embodiments, -Cy^{L3}- is

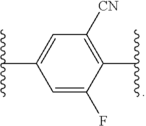 [SMILES: *c1cc(F)c(*)c(C#N)c1]

In some embodiments, -Cy^{L3}- is

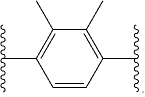 [SMILES: *c1ccc(*)c(C)c1C]

In some embodiments, -Cy^{L3}- is

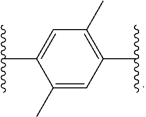 [SMILES: *c1cc(C)c(*)cc1C]

In some embodiments, -Cy^{L3}- is

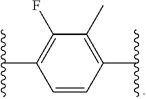 [SMILES: *c1ccc(*)c(F)c1C]

In some embodiments, -Cy^{L3}- is

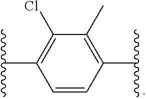 [SMILES: *c1ccc(*)c(Cl)c1C]

In some embodiments, -Cy^{L3}- is

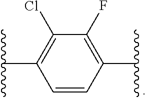 [SMILES: *c1ccc(*)c(Cl)c1F]

In some embodiments, -Cy^{L3}- is

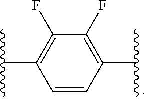 [SMILES: *c1ccc(*)c(F)c1F]

In some embodiments, -Cy^{L3}- is

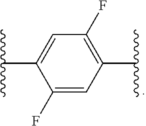 [SMILES: *c1cc(F)c(*)cc1F]

In some embodiments, -Cy^{L3} is

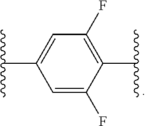 [SMILES: *c1cc(F)c(*)c(F)c1]

In some embodiments, -Cy^{L3}- is

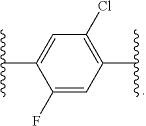 [SMILES: *c1cc(Cl)c(*)cc1F]

In some embodiments, -Cy^{L3}- is

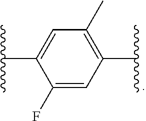 [SMILES: *c1cc(F)c(*)cc1C]

In some embodiments, -Cy^{L3}- is

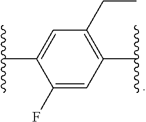 [SMILES: *c1cc(CC)c(*)cc1F]

In some embodiments, -Cy^{L3} is

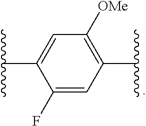 [SMILES: *c1cc(OC)c(*)cc1F]

In some embodiments, -Cy^{L3}- is

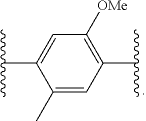 [SMILES: *c1cc(OC)c(*)cc1C]

In some embodiments, -Cy^{L3}- is

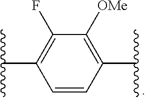 [SMILES: *c1ccc(*)c(OC)c1F]

In some embodiments, -Cy^{L3}- is

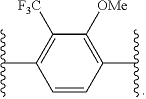 [SMILES: *c1ccc(*)c(C(F)(F)F)c1OC]

In some embodiments, -Cy^{L3}- is

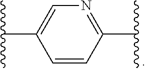 [SMILES: *c1ccc(*)nc1]

In some embodiments, -Cy^{L3}- is

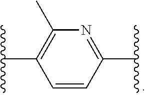 [SMILES: *c1ccc(*)c(C)n1]

In some embodiments, -Cy^{L3}- is

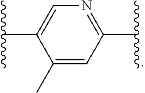 [SMILES: *c1cc(C)c(*)cn1]

In some embodiments, -Cy^{L3}- is

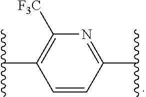 [SMILES: *c1ccc(*)c(C(F)(F)F)n1]

In some embodiments, -Cy^{L3} is

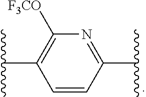 [SMILES: *c1ccc(*)c(OC(F)(F)F)n1]

In some embodiments, -Cy^{L3}- is

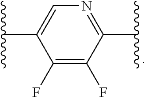 [SMILES: *c1cnc(*)c(F)c1F]

In some embodiments, -Cy^{L3}- is

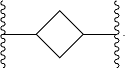 [SMILES: *C1CC(*)C1]

In some embodiments, Cy^{L3}- is

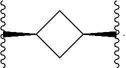 [SMILES: *[C@H]1C[C@@H](*)C1]

In some embodiments, -Cy^{L3}- is

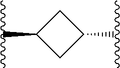 [SMILES: *[C@H]1C[C@H](*)C1]

In some embodiments, -Cy^{L3}- is

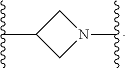 [SMILES: *C1CN(*)C1]

In some embodiments, -Cy^{L3}- is

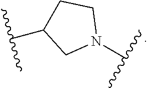 [SMILES: *C1CCN(*)C1]

In some embodiments, -Cy^{L3}- is

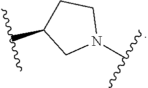 [SMILES: *[C@H]1CCN(*)C1]

In some embodiments, -Cy^{L3}- is

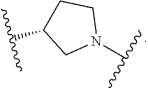 [SMILES: *[C@@H]1CCN(*)C1]

In some embodiments, -Cy^{L3}- is

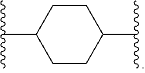 [SMILES: *C1CCC(*)CC1]

In some embodiments, -Cy^{L3}- is

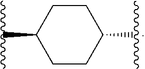 [SMILES: *[C@H]1CC[C@H](*)CC1]

In some embodiments, -Cy^{L3}- is

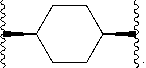 [SMILES: *[C@H]1CC[C@@H](*)CC1]

In some embodiments, -Cy^{L3}- is

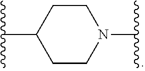 [SMILES: *C1CCN(*)CC1]

In some embodiments, -Cy^{L3}- is

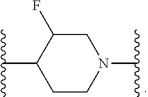 [SMILES: *C1CCN(*)CC1F]

In some embodiments, -Cy^{L3}- is

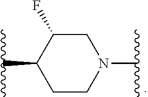 [SMILES: *[C@@H]1CCN(*)C[C@H]1F]

In some embodiments, -Cy^{L3}- is

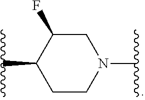 [SMILES: *[C@@H]1CCN(*)C[C@@H]1F]

In some embodiments, -Cy^{L3}- is

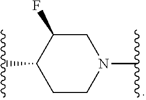 [SMILES: *[C@H]1CCN(*)C[C@@H]1F]

In some embodiments, -Cy^{L3}- is

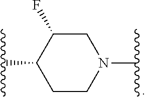 [SMILES: *[C@H]1CCN(*)C[C@H]1F]

In some embodiments, -Cy^{L3}- is

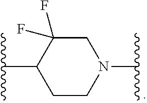 [SMILES: *C1CCN(*)CC1(F)F]

In some embodiments, -Cy^{L3}- is

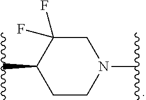 [SMILES: *[C@@H]1CCN(*)CC1(F)F]

In some embodiments, -Cy^{L3} is

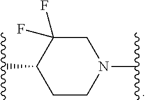 [SMILES: *[C@H]1CCN(*)CC1(F)F]

In some embodiments, -Cy^{L3}- is

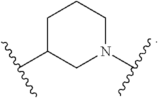 [SMILES: *C1CCCN(*)C1]

In some embodiments, -Cy^{L3}- is

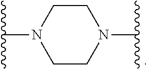 [SMILES: *N1CCN(*)CC1]

In some embodiments, -Cy^{L3}- is

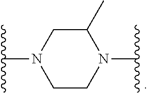 [SMILES: *N1CCN(*)C(C)C1]

In some embodiments, -Cy^{L3}- is

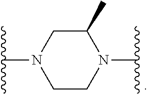 [SMILES: *N1CCN(*)[C@H](C)C1]

In some embodiments, -Cy^{L3}- is

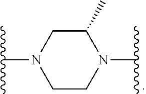 [SMILES: *N1CCN(*)[C@@H](C)C1]

In some embodiments, -Cy^{L3}- is

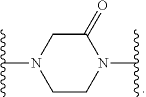 [SMILES: *N1CCN(*)C(=O)C1]

In some embodiments, -Cy^{L3}- is

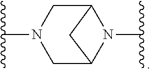 [SMILES: *N1CC2CC(C1)N2*]

In some embodiments, -Cy^{L3}- is

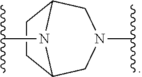 [SMILES: *N1CC2CCC(C1)N2*]

In some embodiments, -Cy^{L3}- is

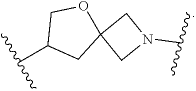 [SMILES: *C1COC2(C1)CN(*)C2]

In some embodiments, -Cy^{L3}- is

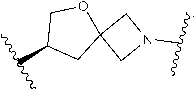 [SMILES: *[C@H]1COC2(C1)CN(*)C2]

In some embodiments, -Cy^{L3}- is

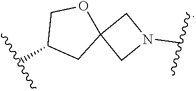 [SMILES: *[C@@H]1COC2(C1)CN(*)C2]

In some embodiments, -Cy^{L3}- is

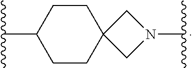 [SMILES: *C1CCC2(CC1)CN(*)C2]

In some embodiments, -Cy^{L3}- is

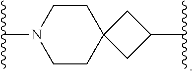 [SMILES: *C1CC2(CCN(*)CC2)C1]

In some embodiments, -Cy^{L3}- is

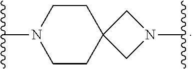 [SMILES: *N1CCC2(CC1)CN(*)C2]

In some embodiments, -Cy^{L3}- is

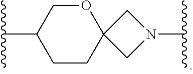 [SMILES: *C1CCC2(CN(*)C2)OC1]

In some embodiments, -Cy^{L3}- is

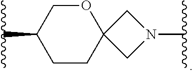 [SMILES: *[C@@H]1CCC2(CN(*)C2)OC1]

In some embodiments, -Cy^{L3}- is

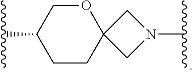 [SMILES: *[C@H]1CCC2(CN(*)C2)OC1]

In some embodiments, -Cy^{L3}- is

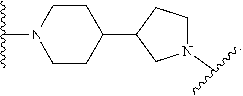 [SMILES: *N1CCC(C2CCN(*)C2)CC1]

In some embodiments, -Cy^{L3} is

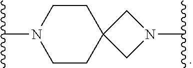 [SMILES: *N1CCC2(CC1)CN(*)C2]

In some embodiments, -Cy^{L3} is

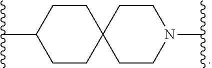 [SMILES: *C1CCC2(CC1)CCN(*)CC2]

In some embodiments, -Cy^{L3}- is

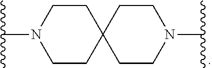 [SMILES: *N1CCC2(CC1)CCN(*)CC2]

In some embodiments, —Cy^{L3}- is F

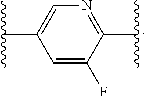 [SMILES: *c1cnc(*)c(F)c1]

In some embodiments, -Cy^{L4}- is an optionally substituted 8-10 membered bicyclic arylenyl. In some embodiments, -Cy^{L4}- is an optionally substituted 3-7 membered saturated or partially unsaturated carbocyclylenyl. In some embodiments, -Cy^{L4}- is an optionally substituted 6-11 membered saturated or partially unsaturated spiro carbocyclylenyl. In some embodiments, -Cy^{L4} is an 8-10 membered bicyclic saturated or partially unsaturated carbocyclylenyl. In some embodiments, -Cy^{L4}- is an optionally substituted a 4-7 membered saturated or partially unsaturated heterocyclylenyl having 1-2 heteroatoms independently selected from nitrogen, oxygen, and sulfur. In some embodiments, -Cy^{L4}- is an optionally substituted 6-11 membered saturated or partially unsaturated spiro heterocyclylenyl having 1-3 heteroatoms independently selected from nitrogen, oxygen, and sulfur. In some embodiments, -Cy^{L4}- is an optionally substituted 8-10 membered bicyclic saturated or partially unsaturated heterocyclylenyl having 1-2 heteroatoms independently selected from nitrogen, oxygen, and sulfur. In some embodiments, -Cy^{L4}- is an optionally substituted 5-6 membered heteroarylenyl having 1-4 heteroatoms independently selected from nitrogen, oxygen. In some embodiments, -Cy^{L4}- is an optionally substituted 8-10 membered bicyclic heteroarylenyl having 1-5 heteroatoms independently selected from nitrogen, oxygen, and sulfur.

In some embodiments, each -Cy^{L1}- is independently a ring selected from phenylenyl, an 8-10 membered bicyclic arylenyl, a 4-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur.

In some embodiments, -Cy^{L4}- is an 8-10 membered bicyclic arylenyl. In some embodiments, -Cy^{L4} is a 3-7 membered saturated or partially unsaturated carbocyclylenyl. In some embodiments, -Cy^{L4}- is a 6-11 membered saturated or partially unsaturated spiro carbocyclylenyl. In some embodiments, -Cy^{L4}- is an 8-10 membered bicyclic saturated or partially unsaturated carbocyclylenyl. In some embodiments, -Cy^{L4}- is a 4-7 membered saturated or partially unsaturated heterocyclylenyl having 1-2 heteroatoms independently selected from nitrogen, oxygen, and sulfur. In some embodiments, -Cy^{L4}- is a 6-11 membered saturated or partially unsaturated spiro heterocyclylenyl having 1-3 heteroatoms independently selected from nitrogen, oxygen, and sulfur. In some embodiments, -Cy^{L4}- is an 8-10 membered bicyclic saturated or partially unsaturated heterocyclylenyl having 1-2 heteroatoms independently selected from nitrogen, oxygen, and sulfur. In some embodiments, -Cy^{L4}- is a 5-6 membered heteroarylenyl having 1-4 heteroatoms independently selected from nitrogen, oxygen. In some embodiments, -Cy^{L4}- is an 8-10 membered bicyclic heteroarylenyl having 1-5 heteroatoms independently selected from nitrogen, oxygen, and sulfur.

In some embodiments, -Cy^{L4}- is an optionally substituted bivalent ring selected from phenylenyl, an 8-10 membered bicyclic arylenyl, a 3-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur.

In some embodiments, -Cy^{L4}- is an optionally substituted bivalent ring selected from phenylenyl, an 8-10 membered bicyclic arylenyl, a 3-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated or partially unsatured heterocyclylenyl having 1-2 heteroatoms independently selected from nitrogen, oxygen, and sulfur, and a 5-6 membered heteroarylenyl having 1-4 heteroatoms independently selected from nitrogen, oxygen, and sulfur.

In some embodiments, -Cy^{L4}- is an optionally substituted phenylenyl. In some embodiments, -Cy^{L4}- is phenylenyl, optionally substituted with halogen (e.g., fluoro or chloro), —CN, C₁₋₆ aliphatic optionally further substituted with halogen (e.g., fluoro or chloro), —S(O)₂R^{∘}, or —OR^{∘}, wherein R^{∘} is hydrogen or C₁₋₆ aliphatic. In some embodiments, -Cy^{L4}- is phenylenyl, optionally substituted with halogen (e.g., fluoro or chloro) or C₁₋₆ aliphatic.

In some embodiments, -Cy^{L4}- is an optionally substituted 5-6 membered saturated or partially unsaturated heterocyclylenyl having 1-2 heteroatoms independently selected from nitrogen, oxygen, and sulfur. In some embodiments, -Cy^{L4}- is an optionally substituted 5 membered saturated or partially unsaturated heterocyclylenyl having 1-2 heteroatoms independently selected from nitrogen, oxygen, and sulfur. In some embodiments, -Cy^{L4}- is an optionally substituted pyrrolidinylenyl. In some embodiments, -Cy^{L4}- is pyrrolidinylenyl, optionally substituted with halogen (e.g., fluoro or chloro), —CN, C₁₋₆ aliphatic optionally further substituted with halogen (e.g., fluoro or chloro), —S(O)₂R^{∘}, or —OR^{∘}, wherein R^{∘} is hydrogen or C₁₋₆ aliphatic. In some embodiments, -Cy^{L4}- is pyrrolidinylenyl, optionally substituted with halogen (e.g., fluoro or chloro) or C₁₋₆ aliphatic. In some embodiments, -Cy^{L4}- is an optionally substituted 6 membered saturated or partially unsaturated heterocyclylenyl having 1-2 heteroatoms independently selected from nitrogen, oxygen, and sulfur. In some embodiments, -Cy^{L4}- is an optionally substituted piperadinylenyl. In some embodiments, -Cy^{L4}- is piperadinylenyl, optionally substituted with halogen (e.g., fluoro or chloro), —CN, C₁₋₆ aliphatic optionally further substituted with halogen (e.g., fluoro or chloro), —S(O)₂R^{∘}, or —OR^{∘}, wherein R^{∘} is hydrogen or C—₆ aliphatic. In some embodiments, -Cy^{L1}- is piperadinylenyl, optionally substituted with halogen (e.g., fluoro or chloro) or C₁₋₆ aliphatic. In some embodiments, -Cy^{L4}- is an optionally substituted piperazinylenyl. In some embodiments, -Cy^{L4}- is piperazinylenyl, optionally substituted with halogen (e.g., fluoro or chloro), —CN, C₁₋₆ aliphatic optionally further substituted with halogen (e.g., fluoro or chloro), —S(O)₂R^{∘}, or —OR^{∘}, wherein R^{∘} is hydrogen or C₁₋₆ aliphatic. In some embodiments, -Cy^{L4}- is piperazinylenyl, optionally substituted with halogen (e.g., fluoro or chloro) or C₁₋₆ aliphatic.

In some embodiments, -Cy^{L4}- is an optionally substituted 5 membered heteroarylenyl having 1-4 heteroatoms independently selected from nitrogen, oxygen. In some embodiments, -Cy^{L4}- is an optionally substituted pyrrolylenyl, pyrazolylenyl, imidazolylenyl, or triazolylenyl. In some embodiments, -Cy^{L4} is pyrrolylenyl, pyrazolylenyl, imidazolylenyl, or triazolylenyl, optionally substituted with halogen (e.g., fluoro or chloro), —CN, C—₆ aliphatic optionally further substituted with halogen (e.g., fluoro or chloro), —S(O)₂R^{∘}, or —OR^{∘}, wherein R^{∘} is hydrogen or C₆ aliphatic. In some embodiments, -Cy^{L4}- is pyrrolylenyl, pyrazolylenyl, imidazolylenyl, or triazolylenyl, optionally substituted with halogen (e.g., fluoro or chloro) or C₁₋₆ aliphatic. In some embodiments, -Cy^{L4}- is an optionally substituted pyrazolylenyl. In some embodiments, -Cy^{L4}- is pyrazolylenyl, optionally substituted with halogen (e.g., fluoro or chloro), —CN, C₁₋₆ aliphatic optionally further substituted with halogen (e.g., fluoro or chloro), —S(O)₂R^{∘}, or —OR^{∘}, wherein R^{∘} is hydrogen or C₆ aliphatic. In some embodiments, -Cy^{L4}- is pyrazolylenyl, optionally substituted with halogen (e.g., fluoro or chloro) or C₁₋₆ aliphatic. In some embodiments, -Cy^{L4}- is an optionally substituted imidazolylenyl. In some embodiments, -Cy^{L4}- is imidazolylenyl, optionally substituted with halogen (e.g., fluoro or chloro), —CN, C₁₋₆ aliphatic optionally further substituted with halogen (e.g., fluoro or chloro), or —OR^{∘}, wherein R^{∘} is hydrogen or C₁₋₆ aliphatic. In some embodiments, -Cy^{L4}- is imidazolylenyl, optionally substituted with halogen (e.g., fluoro or chloro) or C₁₋₆ aliphatic.

In some embodiments, -Cy^{L4}- is an optionally substituted 6 membered heteroarylenyl having 1-4 heteroatoms independently selected from nitrogen, oxygen. In some embodiments, -Cy^{L4}- is an optionally substituted pyridinylenyl, pyridazinylenyl, pyrimidinylenyl, pyrazinylenyl, or triazinylenyl. In some embodiments, -Cy^{L4}- is pyridinylenyl, pyridazinylenyl, pyrimidinylenyl, pyrazinylenyl, or triazinylenyl, optionally substituted with halogen (e.g., fluoro or chloro), —CN, C₁₋₆ aliphatic optionally further substituted with halogen (e.g., fluoro or chloro), —S(O)₂R^{∘}, or —OR^{∘}, wherein R^{∘} is hydrogen or C₁₋₆ aliphatic. In some embodiments, -Cy^{L4}- is pyridinylenyl, pyridazinylenyl, pyrimidinylenyl, pyrazinylenyl, or triazinylenyl, optionally substituted with halogen (e.g., fluoro or chloro) or C₁₋₆ aliphatic. In some embodiments, -Cy^{L4}- is an optionally substituted pyridinylenyl. In some embodiments, -Cy^{L4}- is pyridinylenyl, optionally substituted with halogen (e.g., fluoro or chloro), —CN, C₁₋₆ aliphatic optionally further substituted with halogen (e.g., fluoro or chloro), —S(O)₂R^{∘}, or —OR^{∘}, wherein R^{∘} is hydrogen or C₁₋₆ aliphatic. In some embodiments, -Cy^{L4}- is pyridinylenyl, optionally substituted with halogen (e.g., fluoro or chloro) or C₁₋₆ aliphatic. In some embodiments, -Cy^{L4}- is an optionally substituted pyridazinylenyl. In some embodiments, -Cy^{L4}- is pyridazinylenyl, optionally substituted with halogen (e.g., fluoro or chloro), —CN, C₁₋₆ aliphatic optionally further substituted with halogen (e.g., fluoro or chloro), —S(O)₂R^{∘}, or —OR^{∘}, wherein R^{∘} is hydrogen or C₁₋₆ aliphatic. In some embodiments, -Cy^{L4}- is pyridazinylenyl, optionally substituted with halogen (e.g., fluoro or chloro) or C₁₋₆ aliphatic. In some embodiments, -Cy^{L4}- is an optionally substituted pyridinonylenyl, pyridazinonylenyl, pyrimidinonylenyl, pyrazinonylenyl, or triazinonylenyl. In some embodiments, -Cy^{L4}- is pyridinonylenyl, pyridazinonylenyl, pyrimidinonylenyl, pyrazinonylenyl, or triazinonylenyl, optionally substituted with halogen (e.g., fluoro or chloro), —CN. C₁₋₆ aliphatic optionally further substituted with halogen (e.g., fluoro or chloro), —S(O)₂R^{∘}, or —OR^{∘}, wherein R^{∘} is hydrogen or C₁₋₆ aliphatic. In some embodiments, -Cy^{L4}- is pyridinonylenyl, pyridazinonylenyl, pyrimidinonylenyl, pyrazinonylenyl, or triazinonylenyl, optionally substituted with halogen (e.g., fluoro or chloro) or C₁₋₆ aliphatic. In some embodiments, -Cy^{L4}- is an optionally substituted pyridinonylenyl. In some embodiments, -Cy^{L4}- is pyridinonylenyl, optionally substituted with halogen (e.g., fluoro or chloro), —CN, C₁₋₆ aliphatic optionally further substituted with halogen (e.g., fluoro or chloro), —S(O)₂R^{∘}, or —OR^{∘}, wherein R^{∘} is hydrogen or C₁₋₆ aliphatic. In some embodiments, -Cy^{L4}- is pyridinonylenyl, optionally substituted with halogen (e.g., fluoro or chloro) or C₁₋₆ aliphatic.

In some embodiments, -Cy^{L4}- is an optionally substituted cyclopropyl. In some embodiments, -Cy^{L4} is an optionally substituted 5 membered saturated or partially unsaturated carbocyclylenyl. In some embodiments, -Cy^{L4}- is an optionally substituted cyclopentanylenyl or cyclopentenylenyl. In some embodiments, -Cy^{L4}- is an optionally substituted 6 membered saturated or partially unsaturated carbocyclylenyl. In some embodiments, -Cy^{L4}- is an optionally substituted cyclohexanylenyl or cyclohexenylenyl. In some embodiments, -Cy^{L4} is an optionally substituted naphthalenylenyl.

In some embodiments, -Cy^{L4}- is an optionally substituted 9 membered bicyclic heteroarylenyl having 1-5 heteroatoms independently selected from nitrogen, oxygen, and sulfur. In some embodiments, -Cy^{L4} is an optionally substituted indolylenyl, azaindolylenyl, isoindolylenyl, azaisoindolylenyl, indazolylenyl, azaindazolylenyl, benzimidazolylenyl, or azabenzimidazolylenyl. In some embodiments, -Cy^{L4} is an optionally substituted indolylenyl. In some embodiments, -Cy^{L4}- is an optionally substituted benzothiophenylenyl, benzofuranylenyl, isobenzofuranylenyl, benzoisooxazolylenyl, benzoisothiazolylenyl, benzoxazolylenyl, benzothiazolylenyl, or benzothiadiazolylenyl.

In some embodiments, -Cy^{L4}- is an optionally substituted 8-11 membered saturated or partially unsaturated spiro heterocyclylenyl having 1-3 heteroatoms independently selected from nitrogen, oxygen, and sulfur. In some embodiments, -Cy^{L4}- is an optionally substituted 9-11 membered saturated or partially unsaturated spiro heterocyclylenyl having 1-3 heteroatoms independently selected from nitrogen, oxygen, and sulfur. In some embodiments, -Cy^{L4}- is an optionally substituted 9 membered saturated or partially unsaturated spiro heterocyclylenyl having 1-3 heteroatoms independently selected from nitrogen, oxygen, and sulfur. In some embodiments, -Cy^{L4}- is an optionally substituted 9 membered saturated or partially unsaturated spiro heterocyclylenyl having 1-3 nitrogen heteroatoms. In some embodiments, -Cy^{L4}- is an optionally substituted 4,6-spiro heterocyclylenyl having 1-3 heteroatoms independently selected from nitrogen, oxygen, and sulfur. In some embodiments, -Cy^{L4}- is an optionally substituted

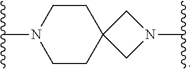 [SMILES: *N1CCC2(CC1)CN(*)C2]

In some embodiments, -Cy^{L4}- is an optionally substituted 10 membered saturated or partially unsaturated spiro heterocyclylenyl having 1-3 heteroatoms independently selected from nitrogen, oxygen, and sulfur. In some embodiments, -Cy^{L4}- is an optionally substituted 10 membered saturated or partially unsaturated spiro heterocyclylenyl having 1-3 nitrogen heteroatoms. In some embodiments, -Cy^{L4}- is an optionally substituted 5,6-spiro heterocyclylenyl having 1-3 heteroatoms independently selected from nitrogen, oxygen, and sulfur.

In some embodiments, -Cy^{L4}- is an optionally substituted 11 membered saturated or partially unsaturated spiro heterocyclylenyl having 1-3 heteroatoms independently selected from nitrogen, oxygen, and sulfur. In some embodiments, -Cy^{L4}- is an optionally substituted 11 membered saturated or partially unsaturated spiro heterocyclylenyl having 1-3 nitrogen heteroatoms. In some embodiments, -Cy^{L4} is an optionally substituted 6,6-spiro heterocyclylenyl having 1-3 heteroatoms independently selected from nitrogen, oxygen, and sulfur. In some embodiments, -Cy^{L4}- is an optionally substituted

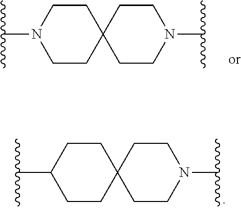 [SMILES: *N1CCC2(CC1)CCN(*)CC2 ; *C1CCC2(CC1)CCN(*)CC2]

In some embodiments, -Cy^{L4}- is optionally substituted

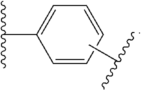

In some embodiments, -Cy^{L4}- is

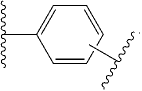

In some embodiments, -Cy^{L4}- is

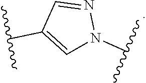 [SMILES: *c1cnn(*)c1]

In some embodiments, -Cy^{L4}- is

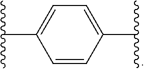 [SMILES: *c1ccc(*)cc1]

In some embodiments, -Cy^{L4}- is

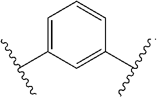 [SMILES: *c1cccc(*)c1]

In some embodiments, -Cy^{L4}- is

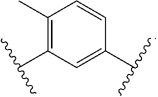 [SMILES: *c1ccc(C)c(*)c1]

In some embodiments, -Cy^{L4}- is

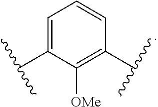 [SMILES: *c1cccc(*)c1OC]

In some embodiments, -Cy^{L4}- is

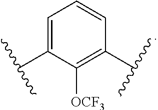 [SMILES: *c1cccc(*)c1OC(F)(F)F]

In some embodiments, -Cy^{L4}- is

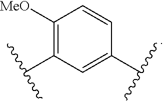 [SMILES: *c1ccc(OC)c(*)c1]

In some embodiments, -Cy^{L4}- is

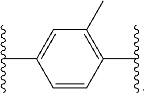 [SMILES: *c1ccc(*)c(C)c1]

In some embodiments, -Cy^{L4}- is

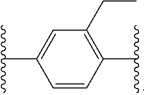 [SMILES: *c1ccc(*)c(CC)c1]

In some embodiments, -Cy^{L4}- is

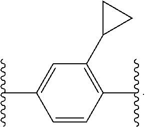 [SMILES: *c1ccc(*)c(C2CC2)c1]

In some embodiments, -Cy^{L4}- is

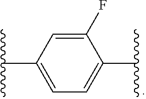 [SMILES: *c1ccc(*)c(F)c1]

In some embodiments, -Cy^{L4}- is

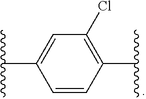 [SMILES: *c1ccc(*)c(Cl)c1]

In some embodiments, -Cy^{L4}- is

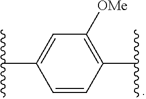 [SMILES: *c1ccc(*)c(OC)c1]

In some embodiments, -Cy^{L4}- is

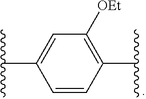 [SMILES: *c1ccc(*)c(OCC)c1]

In some embodiments, -Cy^{L4}- is

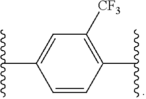 [SMILES: *c1ccc(*)c(C(F)(F)F)c1]

In some embodiments, -Cy^{L4}- is

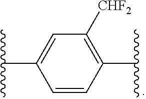 [SMILES: *c1ccc(*)c(C(F)F)c1]

In some embodiments, -Cy^{L4}- is

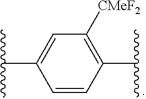

In some embodiments, -Cy^{L4}- is

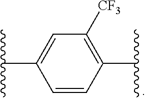 [SMILES: *c1ccc(*)c(C(F)(F)F)c1]

In some embodiments, -Cy^{L4}- is

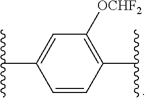 [SMILES: *c1ccc(*)c(OC(F)F)c1]

In some embodiments, -Cy^{L4}- is

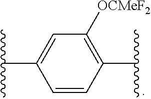

In some embodiments, -Cy^{L4}- is

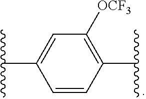 [SMILES: *c1ccc(*)c(OC(F)(F)F)c1]

In some embodiments, -Cy^{L4}- is

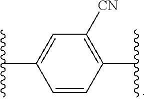 [SMILES: *c1ccc(*)c(C#N)c1]

In some embodiments, -Cy^{L4}- is

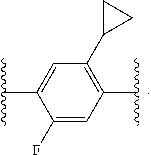 [SMILES: *c1cc(C2CC2)c(*)cc1F]

In some embodiments, -Cy^{L4}- is

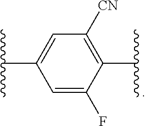 [SMILES: *c1cc(F)c(*)c(C#N)c1]

In some embodiments, -Cy^{L4}- is

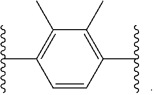 [SMILES: *c1ccc(*)c(C)c1C]

In some embodiments, -Cy^{L4}- is

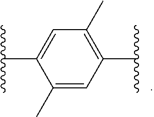 [SMILES: *c1cc(C)c(*)cc1C]

In some embodiments, -Cy^{L4}- is

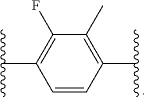 [SMILES: *c1ccc(*)c(F)c1C]

In some embodiments, -Cy^{L4}- is

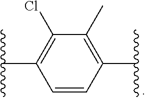 [SMILES: *c1ccc(*)c(Cl)c1C]

In some embodiments, -Cy^{L4}- is

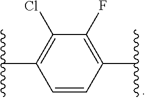 [SMILES: *c1ccc(*)c(Cl)c1F]

In some embodiments, -Cy^{L4}- is

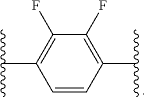 [SMILES: *c1ccc(*)c(F)c1F]

In some embodiments, -Cy^{L4}- is

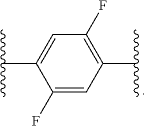 [SMILES: *c1cc(F)c(*)cc1F]

In some embodiments, -Cy^{L4}- is

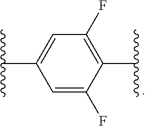 [SMILES: *c1cc(F)c(*)c(F)c1]

In some embodiments, -Cy^{L4}- is

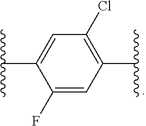 [SMILES: *c1cc(Cl)c(*)cc1F]

In some embodiments, -Cy^{L4}- is

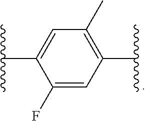 [SMILES: *c1cc(F)c(*)cc1C]

In some embodiments, -Cy^{L4}- is

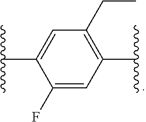 [SMILES: *c1cc(CC)c(*)cc1F]

In some embodiments, -Cy^{L4}- is

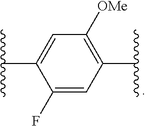 [SMILES: *c1cc(OC)c(*)cc1F]

In some embodiments, -Cy^{L4}- is

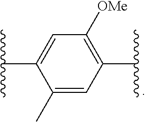 [SMILES: *c1cc(OC)c(*)cc1C]

In some embodiments, -Cy^{L4}- is

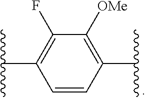 [SMILES: *c1ccc(*)c(OC)c1F]

In some embodiments, -Cy^{L4}- is

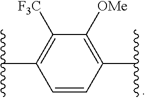 [SMILES: *c1ccc(*)c(C(F)(F)F)c1OC]

In some embodiments, -Cy^{L4}- is

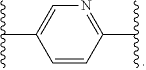 [SMILES: *c1ccc(*)nc1]

In some embodiments, -Cy^{L4}- is

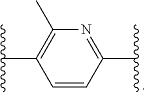 [SMILES: *c1ccc(*)c(C)n1]

In some embodiments, -Cy^{L4}- is

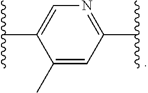 [SMILES: *c1cc(C)c(*)cn1]

In some embodiments, -Cy^{L4}- is

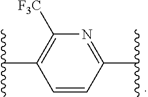 [SMILES: *c1ccc(*)c(C(F)(F)F)n1]

In some embodiments, -Cy^{L4}- is

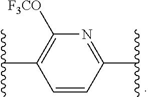 [SMILES: *c1ccc(*)c(OC(F)(F)F)n1]

In some embodiments, -Cy^{L4}- is

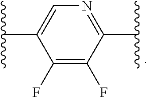 [SMILES: *c1cnc(*)c(F)c1F]

In some embodiments, -Cy^{L4}- is

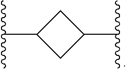 [SMILES: *C1CC(*)C1]

In some embodiments, -Cy^{L4}- is

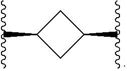 [SMILES: *[C@H]1C[C@@H](*)C1]

In some embodiments, -Cy^{L4}- is

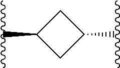 [SMILES: *[C@H]1C[C@H](*)C1]

In some embodiments, -Cy^{L4}- is

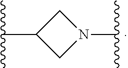 [SMILES: *C1CN(*)C1]

In some embodiments, -Cy^{L4}- is

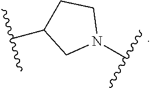 [SMILES: *C1CCN(*)C1]

In some embodiments, -Cy^{L4}- is

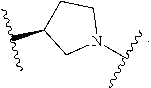 [SMILES: *[C@H]1CCN(*)C1]

In some embodiments, -Cy^{L4}- is

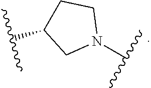 [SMILES: *[C@@H]1CCN(*)C1]

In some embodiments, -Cy^{L4}- is

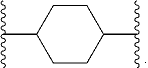 [SMILES: *C1CCC(*)CC1]

In some embodiments, -Cy^{L4}- is

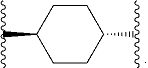 [SMILES: *[C@H]1CC[C@H](*)CC1]

In some embodiments, -Cy^{L4}- is

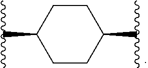 [SMILES: *[C@H]1CC[C@@H](*)CC1]

In some embodiments, -Cy^{L4}- is

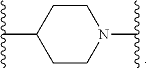 [SMILES: *C1CCN(*)CC1]

In some embodiments, -Cy^{L4}- is

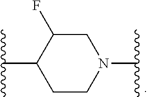 [SMILES: *C1CCN(*)CC1F]

In some embodiments, -Cy^{L4}- is

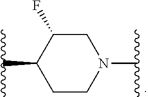 [SMILES: *[C@@H]1CCN(*)C[C@H]1F]

In some embodiments, -Cy^{L4}- is

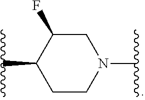 [SMILES: *[C@@H]1CCN(*)C[C@@H]1F]

In some embodiments, -Cy^{L4}- is

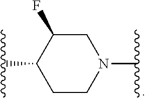 [SMILES: *[C@H]1CCN(*)C[C@@H]1F]

In some embodiments, -Cy^{L4}- is

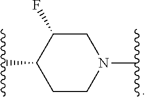 [SMILES: *[C@H]1CCN(*)C[C@H]1F]

In some embodiments, -Cy^{L4}- is

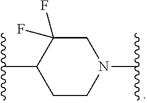 [SMILES: *C1CCN(*)CC1(F)F]

In some embodiments, -Cy^{L4}- is

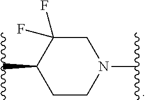 [SMILES: *[C@@H]1CCN(*)CC1(F)F]

In some embodiments, -Cy^{L4}- is

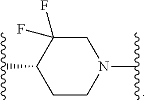 [SMILES: *[C@H]1CCN(*)CC1(F)F]

In some embodiments, -Cy^{L4}- is

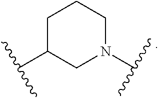 [SMILES: *C1CCCN(*)C1]

In some embodiments, -Cy^{L3}- is

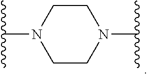 [SMILES: *N1CCN(*)CC1]

In some embodiments, -Cy^{L4}- is

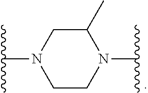 [SMILES: *N1CCN(*)C(C)C1]

In some embodiments, -Cy^{L4}- is

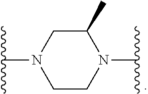 [SMILES: *N1CCN(*)[C@H](C)C1]

In some embodiments, -Cy^{L4}- is

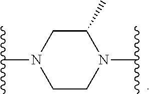 [SMILES: *N1CCN(*)[C@@H](C)C1]

In some embodiments, -Cy^{L1}- is

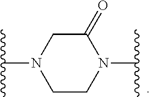 [SMILES: *N1CCN(*)C(=O)C1]

In some embodiments, -Cy^{L4}- is

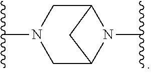 [SMILES: *N1CC2CC(C1)N2*]

In some embodiments, -Cy^{L4}- is

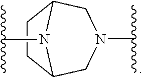 [SMILES: *N1CC2CCC(C1)N2*]

In some embodiments, -Cy^{L4}- is

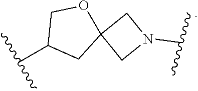 [SMILES: *C1COC2(C1)CN(*)C2]

In some embodiments, -Cy^{L4}- is

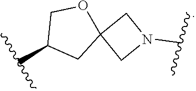 [SMILES: *[C@H]1COC2(C1)CN(*)C2]

In some embodiments, -Cy^{L4}- is

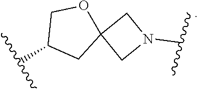 [SMILES: *[C@@H]1COC2(C1)CN(*)C2]

In some embodiments, -Cy^{L4}- is

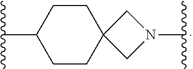 [SMILES: *C1CCC2(CC1)CN(*)C2]

In some embodiments, -Cy^{L4}- is

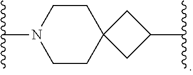 [SMILES: *C1CC2(CCN(*)CC2)C1]

In some embodiments, -Cy^{L4}- is

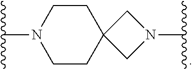 [SMILES: *N1CCC2(CC1)CN(*)C2]

In some embodiments, -Cy^{L4}- is

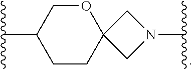 [SMILES: *C1CCC2(CN(*)C2)OC1]

In some embodiments. -Cy^{L4}- is

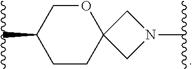 [SMILES: *[C@@H]1CCC2(CN(*)C2)OC1]

In some embodiments, -Cy^{L4} is

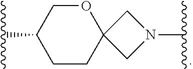 [SMILES: *[C@H]1CCC2(CN(*)C2)OC1]

In some embodiments, -Cy^{L4}- is

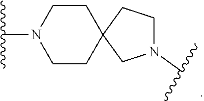 [SMILES: *N1CCC2(CC1)CCN(*)C2]

In some embodiments, -Cy^{L4}- is

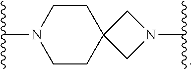 [SMILES: *N1CCC2(CC1)CN(*)C2]

In some embodiments, -Cy^{L4}- is

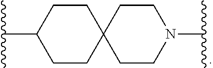 [SMILES: *C1CCC2(CC1)CCN(*)CC2]

In some embodiments, Cy^{L4}- is

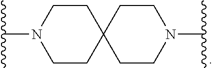 [SMILES: *N1CCC2(CC1)CCN(*)CC2]

In some embodiments, —Cy^{L4}- is

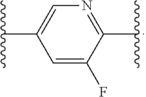 [SMILES: *c1cnc(*)c(F)c1]

In some embodiments, L is:

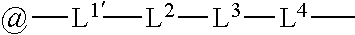

wherein
@, represents the point of attachment to Ring W;
L^{1′} is -Cy^{L1}-;
L² is -Cy^{L2}-;
L³ is -Cy^{L3}-; and
L⁴ is a covalent bond or an optionally substituted bivalent, saturated or partially unsaturated, straight or branched C₁₋₆ hydrocarbon chain, wherein 1-2 methylene units of L⁴ are optionally and independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—.

In some embodiments, L is:

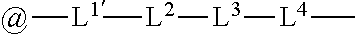

wherein
@, represents the point of attachment to Ring W;
L^{1′} is -Cy^{L1}-;
L² is -Cy^{L2}-;
L³ is -Cy^{L3}-; and
L⁴ is —C(O)—.

In some embodiments, L is:

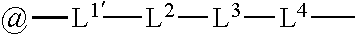

wherein
@ represents the point of attachment to Ring W:
L^{1′} is -Cy^{L1}-;
L² is -Cy^{L2}-:
L³ is -Cy^{L3}-;
L⁴ is a covalent bond or an optionally substituted bivalent, saturated or partially unsaturated, straight or branched C₁₋₆ hydrocarbon chain.

In some embodiments, L is:

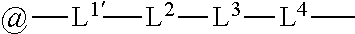

wherein
@ represents the point of attachment to Ring W;
L^{1′} is -Cy^{L1}:
L² is -Cy^{L2}-;
L³ is -Cy^{L3};
L⁴ is an optionally substituted bivalent, saturated or partially unsaturated, straight or branched C₁₋₆ hydrocarbon chain.

In some embodiments, L is:

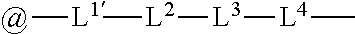

wherein
@ represents the point of attachment to Ring W;
L^{1′} is a covalent bond or an optionally substituted bivalent, saturated or partially unsaturated, straight or branched C₁₋₆ hydrocarbon chain, wherein 1-2 methylene units of L^{1′} are optionally and independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—.
L² is -Cy^{L2}-;
L³ is -Cy^{L3}; and
L⁴ is a covalent bond or an optionally substituted bivalent, saturated or partially unsaturated, straight or branched C₁₋₆ hydrocarbon chain, wherein 1-2 methylene units of L⁴ are optionally and independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—.

In some embodiments, L is:

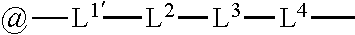

wherein
@ represents the point of attachment to Ring W:
L^{1′} is an optionally substituted bivalent, saturated or partially unsaturated, straight or branched C₁₋₆ hydrocarbon chain, wherein 1-2 methylene units of L^{1′} are optionally and independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—.
L² is -Cy^{L2}-;
L³ is -Cy^{L3}-; and
L⁴ is an optionally substituted bivalent, saturated or partially unsaturated, straight or branched C₁₋₆ hydrocarbon chain, wherein 1-2 methylene units of L⁴ are optionally and independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—.

In some embodiments, L is:

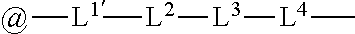

wherein
@ represents the point of attachment to Ring W;
L^{1′} is an optionally substituted bivalent, saturated or partially unsaturated, straight or branched C₁₋₆ hydrocarbon chain;
L² is -Cy^{L2}-:
L³ is -Cy-L³; and
L⁴ is an optionally substituted bivalent, saturated or partially unsaturated, straight or branched C₁₋₆ hydrocarbon chain.

In some embodiments, L is:

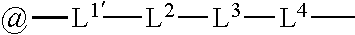

wherein
@, represents the point of attachment to Ring W;
L¹ is -Cy^{L1}-;
L² is a covalent bond or an optionally substituted bivalent, saturated or partially unsaturated, straight or branched C₁₋₆ hydrocarbon chain, wherein 1-2 methylene units of L² are optionally and independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—;
L³ is -Cy^{L3}-; and
L⁴ is a covalent bond or an optionally substituted bivalent, saturated or partially unsaturated, straight or branched C₁₋₆ hydrocarbon chain, wherein 1-2 methylene units of L⁴ are optionally and independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—.

In some embodiments, L is:

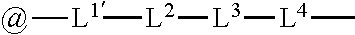

wherein
@ represents the point of attachment to Ring W;
L^{1′} is -Cy^{L1}-;
L² is an optionally substituted bivalent, saturated or partially unsaturated, straight or branched C₁₋₆ hydrocarbon chain, wherein 1-2 methylene units of L² are optionally and independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—;
L³ is -Cy^{L3}-; and
L⁴ is an optionally substituted bivalent, saturated or partially unsaturated, straight or branched C₁₋₆ hydrocarbon chain, wherein 1-2 methylene units of L⁴ are optionally and independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—.

In some embodiments, L is:

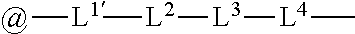

wherein
@ represents the point of attachment to Ring W;
L^{1′} is -Cy^{L1}-;
L² is an optionally substituted bivalent, saturated or partially unsaturated, straight or branched C₁₋₆ hydrocarbon chain;
L³ is -Cy^{L3}; and
L⁴ is an optionally substituted bivalent, saturated or partially unsaturated, straight or branched C₁₋₆ hydrocarbon chain.

In some embodiments, L is:

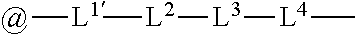

wherein
@ represents the point of attachment to Ring W;
L^{1′} is -Cy^{L1}:
L² is -Cy^{L2};
L³ is a covalent bond or an optionally substituted bivalent, saturated or partially unsaturated, straight or branched C₁₋₆ hydrocarbon chain, wherein 1-2 methylene units of L³ are optionally and independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—; and
L⁴ is -Cy^{L4}-.

In some embodiments, L is:

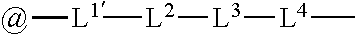

wherein
@ represents the point of attachment to Ring W;
L^{1′} is -Cy^{L1}-:
L² is -Cy^{L2}-;
L³ is an optionally substituted bivalent, saturated or partially unsaturated, straight or branched C₁₋₆ hydrocarbon chain, wherein 1-2 methylene units of L³ are optionally and independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—; and
L⁴ is -Cy^{L4}-.

In some embodiments, L is:

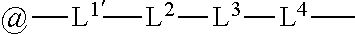

wherein
@ represents the point of attachment to Ring W:
L^{1′} is -Cy^{L1}-;
L² is -Cy^{L2}-;
L³ is an optionally substituted bivalent, saturated or partially unsaturated, straight or branched C₁₋₆ hydrocarbon chain; and
L⁴ is -Cy^{L4}-.

In some embodiments, L is:

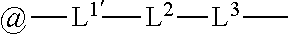

wherein
@ represents the point of attachment to Ring W;
L^{1′} is -Cy^{L1}-;
L² is -Cy^{L2}-; and
L³ is a covalent bond or an optionally substituted bivalent, saturated or partially unsaturated, straight or branched C₁₋₆ hydrocarbon chain, wherein 1-2 methylene units of L³ are optionally and independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—.

In some embodiments, L is:

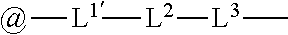

wherein
@ represents the point of attachment to Ring W:
L^{1′} is -Cy^{L1}-;
L² is -Cy^{L2}; and
L³ is a covalent bond or an optionally substituted bivalent, saturated or partially unsaturated, straight or branched C₁₋₆ hydrocarbon chain.

In some embodiments, L is:

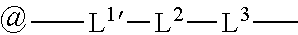

wherein
@ represents the point of attachment to Ring W:
L¹ is -Cy^{L1}-;
L² is -Cy^{L2}-; and
L³ is an optionally substituted bivalent, saturated or partially unsaturated, straight or branched C₁₋₆ hydrocarbon chain.

In some embodiments, L is:

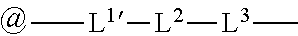

wherein
@ represents the point of attachment to Ring W;
L^{1′} is -Cy^{L1}-:
L² is -Cy^{L2}; and
L³ is -Cy^{L3}-.

In some embodiments, L is:

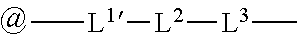

wherein
@ represents the point of attachment to Ring W;
L^{1′} is a covalent bond or an optionally substituted bivalent, saturated or partially unsaturated, straight or branched C₁₋₆ hydrocarbon chain, wherein 1-2 methylene units of L^{1′} are optionally and independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—;
L² is -Cy^{L2}-; and
L³ is a covalent bond or an optionally substituted bivalent, saturated or partially unsaturated, straight or branched C₁₋₆ hydrocarbon chain, wherein 1-2 methylene units of L³ are optionally and independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—.

In some embodiments, L is:

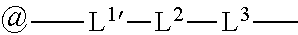

wherein
@ represents the point of attachment to Ring W;
L^{1′} is an optionally substituted bivalent, saturated or partially unsaturated, straight or branched C₁₋₆ hydrocarbon chain, wherein 1-2 methylene units of L^{1′} are optionally and independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—;
L² is -Cy^{L2}-; and
L³ is an optionally substituted bivalent, saturated or partially unsaturated, straight or branched C₁₋₆ hydrocarbon chain, wherein 1-2 methylene units of L³ are optionally and independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—.

In some embodiments, L is:

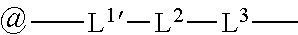

wherein
@ represents the point of attachment to Ring W;
L^{1′} is an optionally substituted bivalent, saturated or partially unsaturated, straight or branched C₁₋₆ hydrocarbon chain;
L² is -Cy^{L2}-; and
L³ is an optionally substituted bivalent, saturated or partially unsaturated, straight or branched C₁₋₆ hydrocarbon chain.

In some embodiments, L is:

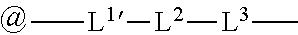

wherein
@ represents the point of attachment to Ring W:
L^{1′} is -Cy^{L1}-;
L² is a covalent bond or an optionally substituted bivalent, saturated or partially unsaturated, straight or branched C₁₋₆ hydrocarbon chain, wherein 1-2 methylene units of L² are optionally and independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—; and
L³ is -Cy^{L3}-;

In some embodiments, L is:

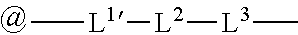

wherein
@ represents the point of attachment to Ring W:
L^{1′} is -Cy^{L1}-;
L² is an optionally substituted bivalent, saturated or partially unsaturated, straight or branched C₁₋₆ hydrocarbon chain, wherein 1-2 methylene units of L² are optionally and independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—; and
L³ is -Cy^{L3}-;

In some embodiments, L is:

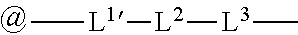

wherein
@ a represents the point of attachment to Ring W:
L^{1′} is -Cy^{L1}-;
L² is an optionally substituted bivalent, saturated or partially unsaturated, straight or branched C₁₋₆ hydrocarbon chain; and
L³ is -Cy^{L3}-.

In some embodiments, L is:

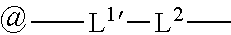

wherein
@ represents the point of attachment to Ring W; L^{1′} is -Cy^{L1}-; and
L² is a covalent bond or an optionally substituted bivalent, saturated or partially unsaturated, straight or branched C₁₋₆ hydrocarbon chain, wherein 1-2 methylene units of L² are optionally and independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—.

In some embodiments, L is:

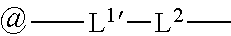

wherein
@ represents the point of attachment to Ring W:
L^{1′} is -Cy^{L1}-; and
L² is an optionally substituted bivalent, saturated or partially unsaturated, straight or branched C₁₋₆ hydrocarbon chain, wherein 1-2 methylene units of L² are optionally and independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—.

In some embodiments, L is:

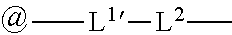

wherein
@ represents the point of attachment to Ring W:
L^{1′} is -Cy^{L1}-; and
L² is an optionally substituted bivalent, saturated or partially unsaturated, straight or branched Cu—hydrocarbon chain.

In some embodiments, L is:

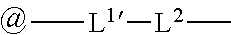

wherein
@ represents the point of attachment to Ring W:
L^{1′} is -Cy^{L1}-; and L² is -Cy^{L2}-.

In some embodiments, each -Cy- is independently an optionally substituted bivalent phenylenyl. In some embodiments, each -Cy- is independently an optionally substituted 8-10 membered bicyclic arylenyl. In some embodiments, each -Cy- is independently an optionally substituted 4-7 membered saturated or partially unsaturated carbocyclylenyl. In some embodiments, each -Cy- is independently an optionally substituted 6-11 membered saturated or partially unsaturated spiro carbocyclylenyl. In some embodiments, each -Cy- is independently an optionally substituted 8-10 membered bicyclic saturated or partially unsaturated carbocyclylenyl. In some embodiments, each -Cy- is independently an optionally substituted 4-7 membered saturated or partially unsaturated heterocyclylenyl having 1-2 heteroatoms independently selected from nitrogen, oxygen, and sulfur. In some embodiments, each -Cy- is independently an optionally substituted 6-11 membered saturated or partially unsaturated spiro heterocyclylenyl having 1-3 heteroatoms independently selected from nitrogen, oxygen, and sulfur. In some embodiments, each -Cy- is independently an optionally substituted 8-10 membered bicyclic saturated or partially unsaturated heterocyclylenyl having 1-2 heteroatoms independently selected from nitrogen, oxygen, and sulfur. In some embodiments, each -Cy- is independently an optionally substituted 5-6 membered heteroarylenyl having 1-4 heteroatoms independently selected from nitrogen, oxygen, and sulfur. In some embodiments, each -Cy- is independently an optionally substituted 8-10 membered bicyclic heteroarylenyl having 1-5 heteroatoms independently selected from nitrogen, oxygen, and sulfur.

In some embodiments, each -Cy- is independently a ring selected from phenylenyl, an 8-10 membered bicyclic arylenyl, a 4-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur.

In some embodiments, each -Cy- is independently bivalent phenylenyl. In some embodiments, each -Cy- is independently an 8-10 membered bicyclic arylenyl. In some embodiments, each -Cy- is independently a 4-7 membered saturated or partially unsaturated carbocyclylenyl. In some embodiments, each -Cy- is independently a 6-11 membered saturated or partially unsaturated spiro carbocyclylenyl. In some embodiments, each -Cy- is independently an 8-10 membered bicyclic saturated or partially unsaturated carbocyclylenyl. In some embodiments, each -Cy- is independently a 4-7 membered saturated or partially unsaturated heterocyclylenyl having 1-2 heteroatoms independently selected from nitrogen, oxygen, and sulfur. In some embodiments, each -Cy- is independently a 6-11 membered saturated or partially unsaturated spiro heterocyclylenyl having 1-3 heteroatoms independently selected from nitrogen, oxygen, and sulfur. In some embodiments, each -Cy- is independently an 8-10 membered bicyclic saturated or partially unsaturated heterocyclylenyl having 1-2 heteroatoms independently selected from nitrogen, oxygen, and sulfur. In some embodiments, each -Cy- is independently a 5-6 membered heteroarylenyl having 1-4 heteroatoms independently selected from nitrogen, oxygen, and sulfur. In some embodiments, each -Cy- is independently an 8-10 membered bicyclic heteroarylenyl having 1-5 heteroatoms independently selected from nitrogen, oxygen, and sulfur.

In some embodiments, each -Cy- is independently an optionally substituted bivalent ring selected from phenylenyl, an 8-10 membered bicyclic arylenyl, a 3-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur.

In some embodiments, each -Cy- is independently an optionally substituted bivalent ring selected from phenylenyl, an 8-10 membered bicyclic arylenyl, a 3-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated or partially unsatured heterocyclylenyl having 1-2 heteroatoms independently selected from nitrogen, oxygen, and sulfur, and a 5-6 membered heteroarylenyl having 1-4 heteroatoms independently selected from nitrogen, oxygen, and sulfur.

In some embodiments, -Cy- is substituted with halogen. In some embodiments, -Cy- is substituted with —CN. In some embodiments, -Cy- is substituted with C₁₋₆ alkyl (e.g., methyl, ethyl, isopropyl). In some embodiments, -Cy- is substituted with C₃₋₆ cycloalkyl. In some embodiments, -Cy- is substituted with C₁₋₆ haloalkyl (e.g., —CHF₂, —CF₃). In some embodiments, -Cy- is substituted with —OC₁₋₆ alkyl (e.g., —OMe, —OEt). In some embodiments, -Cy- is substituted with —OC₁₋₆ haloalkyl (e.g., —OCHF₂, —OCF₃).

In some embodiments, -Cy- is substituted with methyl. In some embodiments, -Cy- is substituted with ethyl. In some embodiments, -Cy- is substituted with cyclopropyl. In some embodiments, -Cy- is substituted with —CHF₂. In some embodiments, -Cy- is substituted with —CMeF₂. In some embodiments, -Cy- is substituted with —CF₃. In some embodiments, -Cy- is substituted with —OCHF₂. In some embodiments, -Cy- is substituted with —OCMeF₂. In some embodiments, -Cy- is substituted with —OCF₃. In some embodiments, -Cy- is substituted with —C(Me)OH. In some embodiments, -Cy- is substituted with oxo. In some embodiments, -Cy- is substituted with fluoro. In some embodiments, -Cy- is substituted with geminal difluoro. In some embodiments, -Cy- is substituted with —OH. In some embodiments, -Cy- is substituted with —OMe. In some embodiments, -Cy- is substituted with —OEt. In some embodiments, -Cy- is substituted with —NR₂.

In some embodiments, -Cy- is phenylenyl, optionally substituted with halogen (e.g., fluoro or chloro), —CN, C₁₋₆ aliphatic optionally further substituted with halogen (e.g., fluoro or chloro), —S(O)₂R^{∘}, or —OR^{∘}, wherein R^{∘} is hydrogen or C₁₋₆ aliphatic. In some embodiments, -Cy- is phenylenyl, optionally substituted with halogen (e.g., fluoro or chloro) or C₆ aliphatic.

In some embodiments, -Cy- is an optionally substituted 5 membered saturated or partially unsaturated heterocyclylenyl having 1-2 heteroatoms independently selected from nitrogen, oxygen, and sulfur. In some embodiments, -Cy- is an optionally substituted pyrrolidinylenyl. In some embodiments, -Cy- is is pyrrolidinylenyl, optionally substituted with halogen (e.g., fluoro or chloro), —CN. C₁₋₆ aliphatic optionally further substituted with halogen (e.g., fluoro or chloro), —S(O)₂R^{∘}, or —OR^{∘}, wherein R^{∘} is hydrogen or C₁₋₆ aliphatic. In some embodiments, -Cy- is pyrrolidinylenyl, optionally substituted with halogen (e.g., fluoro or chloro) or C₁₋₆ aliphatic. In some embodiments, -Cy^{L4}- is an optionally substituted 6 membered saturated or partially unsaturated heterocyclylenyl having 1-2 heteroatoms independently selected from nitrogen, oxygen, and sulfur. In some embodiments, -Cy- is an optionally substituted piperadinylenyl. In some embodiments, -Cy- is piperadinylenyl, optionally substituted with halogen (e.g., fluoro or chloro), —CN, C—₆ aliphatic optionally further substituted with halogen (e.g., fluoro or chloro), —S(O)₂R^{∘}, or —OR^{∘}, wherein R^{∘} is hydrogen or C₁₋₆ aliphatic. In some embodiments, -Cy- is piperadinylenyl, optionally substituted with halogen (e.g., fluoro or chloro) or C₁₋₆ aliphatic. In some embodiments, -Cy- is an optionally substituted piperazinylenyl. In some embodiments, -Cy- is piperazinylenyl, optionally substituted with halogen (e.g., fluoro or chloro), —CN, C₁₋₆ aliphatic optionally further substituted with halogen (e.g., fluoro or chloro), —S(O)₂R^{∘}, or —OR^{∘}, wherein R^{∘} is hydrogen or C₁₋₆ aliphatic. In some embodiments, -Cy- is piperazinylenyl, optionally substituted with halogen (e.g., fluoro or chloro) or C₁₋₆ aliphatic.

In some embodiments, -Cy- is an optionally substituted 5 membered heteroarylenyl having 1-4 heteroatoms independently selected from nitrogen, oxygen. In some embodiments, -Cy- is an optionally substituted pyrrolylenyl, pyrazolylenyl, imidazolylenyl, or triazolylenyl. In some embodiments, -Cy- is pyrrolylenyl, pyrazolylenyl, imidazolylenyl, or triazolylenyl, optionally substituted with halogen (e.g., fluoro or chloro), —CN, C₁₋₆ aliphatic optionally further substituted with halogen (e.g., fluoro or chloro), —S(O)₂R^{∘}, or —OR^{∘}, wherein R^{∘} is hydrogen or C₁₋₆ aliphatic. In some embodiments, -Cy- is pyrrolylenyl, pyrazolylenyl, imidazolylenyl, or triazolylenyl, optionally substituted with halogen (e.g., fluoro or chloro) or C₁₋₆ aliphatic. In some embodiments, -Cy- is an optionally substituted pyrazolylenyl. In some embodiments, -Cy- is pyrazolylenyl, optionally substituted with halogen (e.g., fluoro or chloro), —CN. C₁₋₆ aliphatic optionally further substituted with halogen (e.g., fluoro or chloro), —S(O)₂R^{∘}, or —OR^{∘}, wherein R^{∘} is hydrogen or C₁₋₆ aliphatic. In some embodiments, -Cy- is pyrazolylenyl, optionally substituted with halogen (e.g., fluoro or chloro) or C₁₋₆ aliphatic. In some embodiments, -Cy- is an optionally substituted imidazolylenyl. In some embodiments, -Cy- is imidazolylenyl, optionally substituted with halogen (e.g., fluoro or chloro), —CN, C₁₋₆ aliphatic optionally further substituted with halogen (e.g., fluoro or chloro), or —OR^{∘}, wherein R^{∘} is hydrogen or C₁₋₆ aliphatic. In some embodiments, -Cy- is imidazolylenyl, optionally substituted with halogen (e.g., fluoro or chloro) or C₁₋₆ aliphatic.

In some embodiments, -Cy- is an optionally substituted 6 membered heteroarylenyl having 1-4 heteroatoms independently selected from nitrogen, oxygen. In some embodiments, -Cy- is an optionally substituted pyridinylenyl, pyridazinylenyl, pyrimidinylenyl, pyrazinylenyl, or triazinylenyl. In some embodiments, -Cy- is pyridinylenyl, pyridazinylenyl, pyrimidinylenyl, pyrazinylenyl, or triazinylenyl, optionally substituted with halogen (e.g., fluoro or chloro), —CN, C₁₋₆ aliphatic optionally further substituted with halogen (e.g., fluoro or chloro), —S(O)₂R^{∘}, or —OR^{∘}, wherein R^{∘} is hydrogen or C₁₋₆ aliphatic. In some embodiments, -Cy- is pyridinylenyl, pyridazinylenyl, pyrimidinylenyl, pyrazinylenyl, or triazinylenyl, optionally substituted with halogen (e.g., fluoro or chloro) or C₁₋₆ aliphatic. In some embodiments, -Cy- is an optionally substituted pyridinylenyl. In some embodiments, -Cy- is pyridinylenyl, optionally substituted with halogen (e.g., fluoro or chloro), —CN, C₁₋₆ aliphatic optionally further substituted with halogen (e.g., fluoro or chloro), —S(O)₂R^{∘}, or —OR^{∘}, wherein R^{∘} is hydrogen or C₁₋₆ aliphatic. In some embodiments, -Cy- is pyridinylenyl, optionally substituted with halogen (e.g., fluoro or chloro) or C₁₋₆ aliphatic. In some embodiments, -Cy- is an optionally substituted pyridazinylenyl. In some embodiments, -Cy- is pyridazinylenyl, optionally substituted with halogen (e.g., fluoro or chloro), —CN. C₁₋₆ aliphatic optionally further substituted with halogen (e.g., fluoro or chloro), —S(O)₂R^{∘}, or —OR^{∘}, wherein R^{∘} is hydrogen or C₁₋₆ aliphatic. In some embodiments, -Cy- is pyridazinylenyl, optionally substituted with halogen (e.g., fluoro or chloro) or C₁₋₆ aliphatic. In some embodiments, -Cy- is an optionally substituted pyridinonylenyl, pyridazinonylenyl, pyrimidinonylenyl, pyrazinonylenyl, or triazinonylenyl. In some embodiments, -Cy- is pyridinonylenyl, pyridazinonylenyl, pyrimidinonylenyl, pyrazinonylenyl, or triazinonylenyl, optionally substituted with halogen (e.g., fluoro or chloro), —CN. C₁₋₆ aliphatic optionally further substituted with halogen (e.g., fluoro or chloro), —S(O)₂R^{∘}, or —OR^{∘}, wherein R^{∘} is hydrogen or C₁₋₆ aliphatic. In some embodiments, -Cy- is pyridinonylenyl, pyridazinonylenyl, pyrimidinonylenyl, pyrazinonylenyl, or triazinonylenyl, optionally substituted with halogen (e.g., fluoro or chloro) or C₁₋₆ aliphatic. In some embodiments, -Cy- is an optionally substituted pyridinonylenyl. In some embodiments, -Cy- is pyridinonylenyl, optionally substituted with halogen (e.g., fluoro or chloro), —CN, C₁₋₆ aliphatic optionally further substituted with halogen (e.g., fluoro or chloro), —S(O)₂R^{∘}, or —OR^{∘}, wherein R^{∘} is hydrogen or C₁₋₆ aliphatic. In some embodiments, -Cy- is pyridinonylenyl, optionally substituted with halogen (e.g., fluoro or chloro) or C₁₋₆ aliphatic.

In some embodiments, -Cy- is an optionally substituted cyclopropyl. In some embodiments, -Cy^{L4}— is an optionally substituted 5 membered saturated or partially unsaturated carbocyclylenyl. In some embodiments, -Cy- is an optionally substituted cyclopentanylenyl or cyclopentenylenyl. In some embodiments, -Cy- is an optionally substituted 6 membered saturated or partially unsaturated carbocyclylenyl. In some embodiments, -Cy- is an optionally substituted cyclohexanylenyl or cyclohexenylenyl. In some embodiments, -Cy- is an optionally substituted naphthalenylenyl.

In some embodiments, -Cy- is an optionally substituted 9 membered bicyclic heteroarylenyl having 1-5 heteroatoms independently selected from nitrogen, oxygen, and sulfur. In some embodiments, -Cy- is an optionally substituted indolylenyl, azaindolylenyl, isoindolylenyl, azaisoindolylenyl, indazolylenyl, azaindazolylenyl, benzimidazolylenyl, or azabenzimidazolylenyl. In some embodiments, -Cy- is an optionally substituted indolylenyl. In some embodiments, -Cy- is an optionally substituted benzothiophenylenyl, benzofuranylenyl, isobenzofuranylenyl, benzoisooxazolylenyl, benzoisothiazolylenyl, benzoxazolylenyl, benzothiazolylenyl, or benzothiadiazolylenyl.

In some embodiments, -Cy- is an optionally substituted 9-11 membered saturated or partially unsaturated spiro heterocyclylenyl having 1-3 heteroatoms independently selected from nitrogen, oxygen, and sulfur. In some embodiments, -Cy- is an optionally substituted 9 membered saturated or partially unsaturated spiro heterocyclylenyl having 1-3 heteroatoms independently selected from nitrogen, oxygen, and sulfur. In some embodiments, -Cy- is an optionally substituted 9 membered saturated or partially unsaturated spiro heterocyclylenyl having 1-3 nitrogen heteroatoms. In some embodiments, -Cy- is an optionally substituted 4,6-spiro heterocyclylenyl having 1-3 heteroatoms independently selected from nitrogen, oxygen, and sulfur. In some embodiments, -Cy- is an optionally substituted

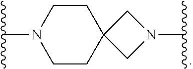 [SMILES: *N1CCC2(CC1)CN(*)C2]

In some embodiments, -Cy- is an optionally substituted 10 membered saturated or partially unsaturated spiro heterocyclylenyl having 1-3 heteroatoms independently selected from nitrogen, oxygen, and sulfur. In some embodiments, -Cy- is an optionally substituted 10 membered saturated or partially unsaturated spiro heterocyclylenyl having 1-3 nitrogen heteroatoms. In some embodiments, -Cy- is an optionally substituted 5,6-spiro heterocyclylenyl having 1-3 heteroatoms independently selected from nitrogen, oxygen, and sulfur.

In some embodiments, -Cy- is an optionally substituted 11 membered saturated or partially unsaturated spiro heterocyclylenyl having 1-3 heteroatoms independently selected from nitrogen, oxygen, and sulfur. In some embodiments, -Cy- is an optionally substituted 11 membered saturated or partially unsaturated spiro heterocyclylenyl having 1-3 nitrogen heteroatoms. In some embodiments, -Cy- is an optionally substituted 6,6-spiro heterocyclylenyl having 1-3 heteroatoms independently selected from nitrogen, oxygen, and sulfur.

In some embodiments, -Cy- is

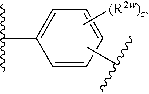

wherein R^{2w} and z are defined above and as described herein.

In some embodiments, -Cy- is

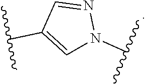 [SMILES: *c1cnn(*)c1]

In some embodiments, -Cy- is

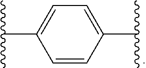 [SMILES: *c1ccc(*)cc1]

In some embodiments, -Cy- is

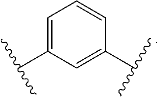 [SMILES: *c1cccc(*)c1]

In some embodiments, -Cy- is

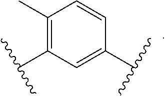 [SMILES: *c1ccc(C)c(*)c1]

In some embodiments, -Cy- is

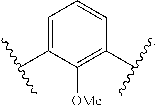 [SMILES: *c1cccc(*)c1OC]

In some embodiments, -Cy- is

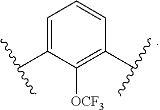 [SMILES: *c1cccc(*)c1OC(F)(F)F]

In some embodiments, -Cy- is

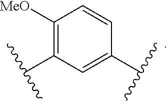 [SMILES: *c1ccc(OC)c(*)c1]

In some embodiments. -Cy- is

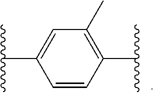 [SMILES: *c1ccc(*)c(C)c1]

In some embodiments, -Cy- is

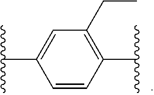 [SMILES: *c1ccc(*)c(CC)c1]

In some embodiments, -Cy- is

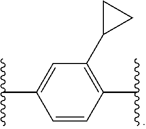 [SMILES: *c1ccc(*)c(C2CC2)c1]

In some embodiments, -Cy- is

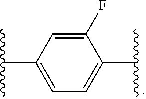 [SMILES: *c1ccc(*)c(F)c1]

In some embodiments, -Cy- is

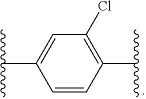 [SMILES: *c1ccc(*)c(Cl)c1]

In some embodiments, -Cy- is

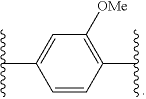 [SMILES: *c1ccc(*)c(OC)c1]

In some embodiments, -Cy- is

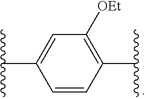 [SMILES: *c1ccc(*)c(OCC)c1]

In some embodiments, -Cy- is

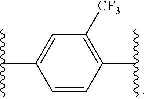 [SMILES: *c1ccc(*)c(C(F)(F)F)c1]

In some embodiments, -Cy- is

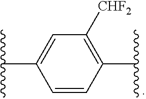 [SMILES: *c1ccc(*)c(C(F)F)c1]

In some embodiments, -Cy- is

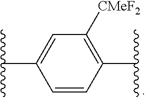

In some embodiments, -Cy- is

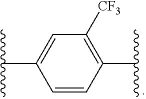 [SMILES: *c1ccc(*)c(C(F)(F)F)c1]

In some embodiments, -Cy- is

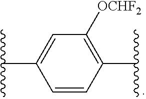 [SMILES: *c1ccc(*)c(OC(F)F)c1]

In some embodiments, -Cy- is

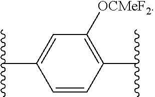

In some embodiments, -Cy- is

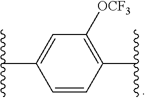 [SMILES: *c1ccc(*)c(OC(F)(F)F)c1]

In some embodiments, -Cy- is

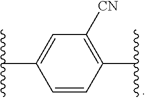 [SMILES: *c1ccc(*)c(C#N)c1]

In some embodiments, -Cy- is

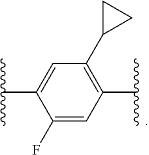 [SMILES: *c1cc(C2CC2)c(*)cc1F]

In some embodiments, -Cy- is

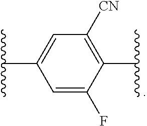 [SMILES: *c1cc(F)c(*)c(C#N)c1]

In some embodiments, -Cy- is

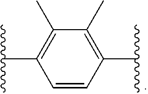 [SMILES: *c1ccc(*)c(C)c1C]

In some embodiments, -Cy- is

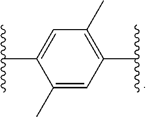 [SMILES: *c1cc(C)c(*)cc1C]

In some embodiments, -Cy- is

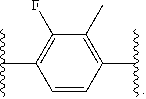 [SMILES: *c1ccc(*)c(F)c1C]

In some embodiments, -Cy- is

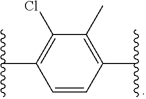 [SMILES: *c1ccc(*)c(Cl)c1C]

In some embodiments, -Cy- is

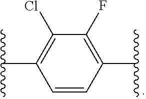 [SMILES: *c1ccc(*)c(Cl)c1F]

In some embodiments, -Cy- is

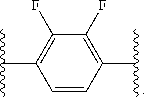 [SMILES: *c1ccc(*)c(F)c1F]

In some embodiments, -Cy- is

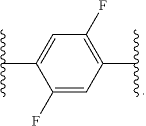 [SMILES: *c1cc(F)c(*)cc1F]

In some embodiments, -Cy- is

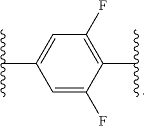 [SMILES: *c1cc(F)c(*)c(F)c1]

In some embodiments, -Cy- is

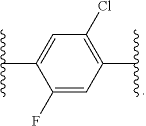 [SMILES: *c1cc(Cl)c(*)cc1F]

In some embodiments, -Cy- is

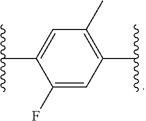 [SMILES: *c1cc(F)c(*)cc1C]

In some embodiments, -Cy- is

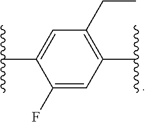 [SMILES: *c1cc(CC)c(*)cc1F]

In some embodiments, -Cy- is

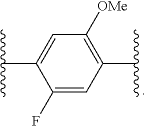 [SMILES: *c1cc(OC)c(*)cc1F]

In some embodiments, -Cy- is

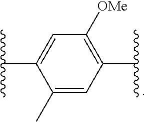 [SMILES: *c1cc(OC)c(*)cc1C]

In some embodiments, -Cy- is

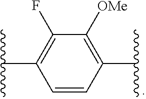 [SMILES: *c1ccc(*)c(OC)c1F]

In some embodiments, -Cy- is

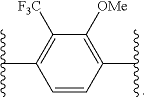 [SMILES: *c1ccc(*)c(C(F)(F)F)c1OC]

In some embodiments, -Cy- is

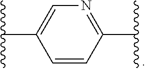 [SMILES: *c1ccc(*)nc1]

In some embodiments, -Cy- is

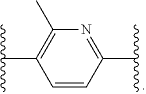 [SMILES: *c1ccc(*)c(C)n1]

In some embodiments, -Cy- is

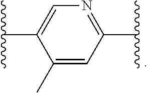 [SMILES: *c1cc(C)c(*)cn1]

In some embodiments, -Cy- is

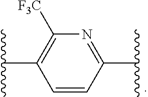 [SMILES: *c1ccc(*)c(C(F)(F)F)n1]

In some embodiments, -Cy- is

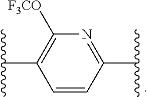 [SMILES: *c1ccc(*)c(OC(F)(F)F)n1]

In some embodiments, -Cy- is

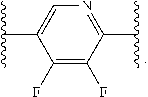 [SMILES: *c1cnc(*)c(F)c1F]

In some embodiments, -Cy- is

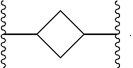 [SMILES: *C1CC(*)C1]

In some embodiments, -Cy- is

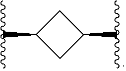 [SMILES: *[C@H]1C[C@@H](*)C1]

In some embodiments, -Cy- is

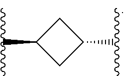 [SMILES: *[C@H]1C[C@H](*)C1]

In some embodiments, -Cy- is

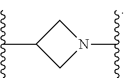 [SMILES: *C1CN(*)C1]

In some embodiments, -Cy- is

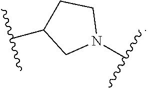 [SMILES: *C1CCN(*)C1]

In some embodiments -Cy- is

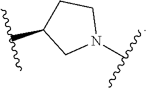 [SMILES: *[C@H]1CCN(*)C1]

In some embodiments, -Cy- is

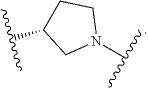 [SMILES: *[C@@H]1CCN(*)C1]

In some embodiments, -Cy- is

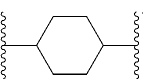 [SMILES: *C1CCC(*)CC1]

In some embodiments, -Cy- is

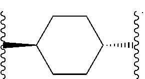 [SMILES: *[C@H]1CC[C@H](*)CC1]

In some embodiments, -Cy- is

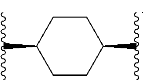 [SMILES: *[C@H]1CC[C@@H](*)CC1]

In some embodiments, -Cy- is

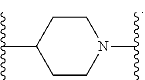 [SMILES: *C1CCN(*)CC1]

In some embodiments, -Cy- is

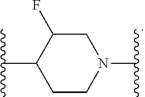 [SMILES: *C1CCN(*)CC1F]

In some embodiments, -Cy- is

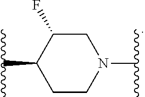 [SMILES: *[C@@H]1CCN(*)C[C@H]1F]

In some embodiments, -Cy- is

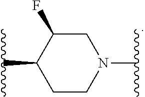 [SMILES: *[C@@H]1CCN(*)C[C@@H]1F]

In some embodiments, -Cy- is

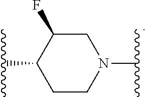 [SMILES: *[C@H]1CCN(*)C[C@@H]1F]

In some embodiments, -Cy- is

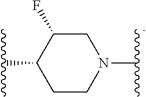 [SMILES: *[C@H]1CCN(*)C[C@H]1F]

In some embodiments, -Cy- is

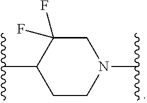 [SMILES: *C1CCN(*)CC1(F)F]

In some embodiments, -Cy- is

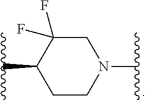 [SMILES: *[C@@H]1CCN(*)CC1(F)F]

In some embodiments, -Cy- is

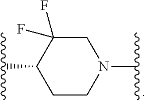 [SMILES: *[C@H]1CCN(*)CC1(F)F]

In some embodiments, -Cy- is

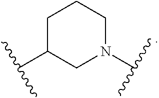 [SMILES: *C1CCCN(*)C1]

In some embodiments, -Cy- is

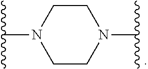 [SMILES: *N1CCN(*)CC1]

In some embodiments, -Cy- is

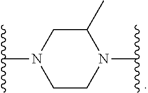 [SMILES: *N1CCN(*)C(C)C1]

In some embodiments, -Cy- is

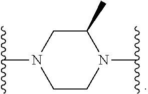 [SMILES: *N1CCN(*)[C@H](C)C1]

In some embodiments, -Cy- is

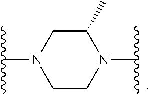 [SMILES: *N1CCN(*)[C@@H](C)C1]

In some embodiments, -Cy- is

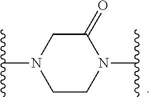 [SMILES: *N1CCN(*)C(=O)C1]

In some embodiments, -Cy- is

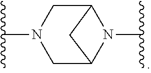 [SMILES: *N1CC2CC(C1)N2*]

In some embodiments, -Cy- is

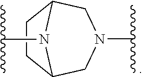 [SMILES: *N1CC2CCC(C1)N2*]

In some embodiments, -Cy- is

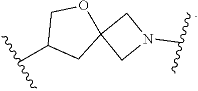 [SMILES: *C1COC2(C1)CN(*)C2]

In some embodiments, -Cy- is

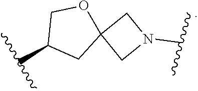 [SMILES: *[C@H]1COC2(C1)CN(*)C2]

In some embodiments, -Cy- is

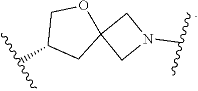 [SMILES: *[C@@H]1COC2(C1)CN(*)C2]

In some embodiments, -Cy- is

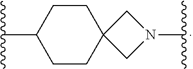 [SMILES: *C1CCC2(CC1)CN(*)C2]

In some embodiments, -Cy- is

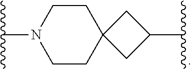 [SMILES: *C1CC2(CCN(*)CC2)C1]

In some embodiments, -Cy- is

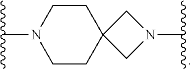 [SMILES: *N1CCC2(CC1)CN(*)C2]

In some embodiments, -Cy- is

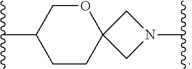 [SMILES: *C1CCC2(CN(*)C2)OC1]

In some embodiments, -Cy- is

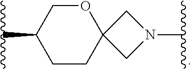 [SMILES: *[C@@H]1CCC2(CN(*)C2)OC1]

In some embodiments, -Cy- is

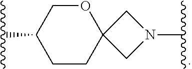 [SMILES: *[C@H]1CCC2(CN(*)C2)OC1]

In some embodiments, -Cy- is

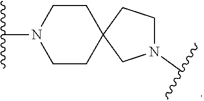 [SMILES: *N1CCC2(CC1)CCN(*)C2]

In some embodiments, -Cy- is

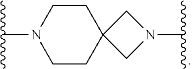 [SMILES: *N1CCC2(CC1)CN(*)C2]

In some embodiments, -Cy- is

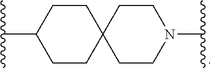 [SMILES: *C1CCC2(CC1)CCN(*)CC2]

In some embodiments, -Cy- is

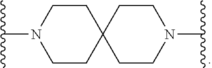 [SMILES: *N1CCC2(CC1)CCN(*)CC2]

In some embodiments -Cy- is

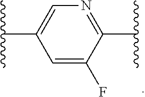 [SMILES: *c1cnc(*)c(F)c1]

In some embodiments, -Cy- is selected from those depicted in Table 1, below.

In some embodiments, r is 3. In some embodiments, r is 1. In some embodiments, r is 2. In some embodiments, r is 3. In some embodiments, r is 4. In some embodiments, r is 5. In some embodiments, r is 6. In some embodiments, r is 7. In some embodiments, r is 8. In some embodiments, r is 9. In some embodiments, r is 10.

In some embodiments, r is selected from those depicted in Table 1, below.

In some embodiments, L is

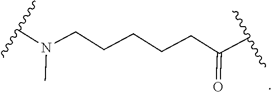 [SMILES: *C(=O)CCCCCN(*)C]

In some embodiments, L is

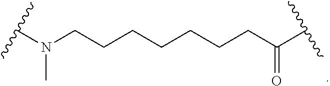 [SMILES: *C(=O)CCCCCCCN(*)C]

In some embodiments, L is

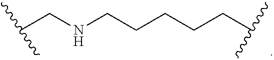 [SMILES: *CCCCCNC*]

In some embodiments, L is

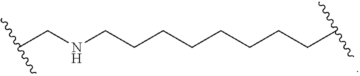 [SMILES: *CCCCCCCCNC*]

In some embodiments, L is

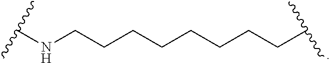 [SMILES: *CCCCCCCCN*]

In some embodiments, L is

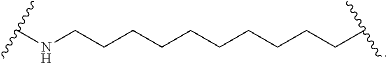 [SMILES: *CCCCCCCCCCN*]

In some embodiments, L is

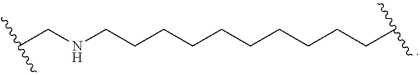 [SMILES: *CCCCCCCCCCNC*]

In some embodiments, L is

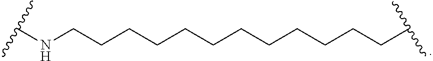 [SMILES: *CCCCCCCCCCCCN*]

In some embodiments, L is

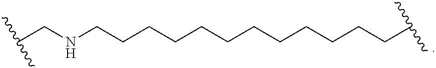 [SMILES: *CCCCCCCCCCCCNC*]

In some embodiments, L is

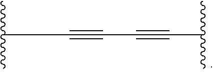 [SMILES: *C#CC#CC*]

In some embodiments, L is

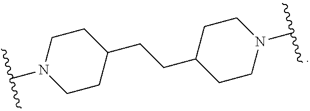 [SMILES: *N1CCC(CCC2CCN(*)CC2)CC1]

In some embodiments, L is

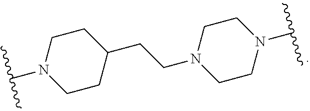 [SMILES: *N1CCC(CCN2CCN(*)CC2)CC1]

In some embodiments, L is

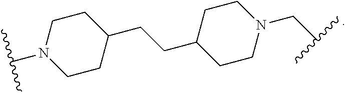 [SMILES: *CN1CCC(CCC2CCN(*)CC2)CC1]

In some embodiments, L is

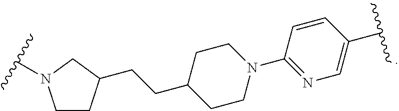 [SMILES: *c1ccc(N2CCC(CCC3CCN(*)C3)CC2)nc1]

In some embodiments, L is

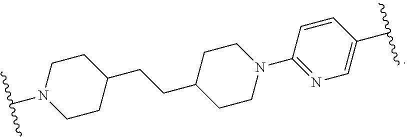 [SMILES: *c1ccc(N2CCC(CCC3CCN(*)CC3)CC2)nc1]

In some embodiments, L is

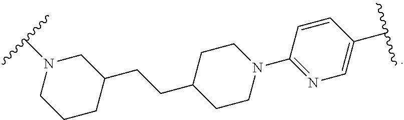 [SMILES: *c1ccc(N2CCC(CCC3CCCN(*)C3)CC2)nc1]

In some embodiments, L is

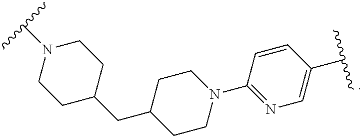 [SMILES: *c1ccc(N2CCC(CC3CCN(*)CC3)CC2)nc1]

In some embodiments, L is

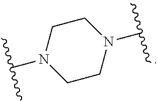 [SMILES: *N1CCN(*)CC1]

In some embodiments, L is

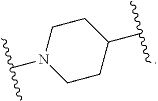 [SMILES: *C1CCN(*)CC1]

In some embodiments, L is

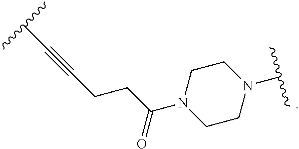 [SMILES: *C#CCCC(=O)N1CCN(*)CC1]

In some embodiments, L is

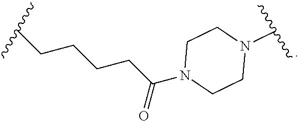 [SMILES: *CCCCC(=O)N1CCN(*)CC1]

In some embodiments, L is

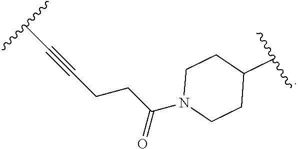 [SMILES: *C#CCCC(=O)N1CCC(*)CC1]

In some embodiments, L is

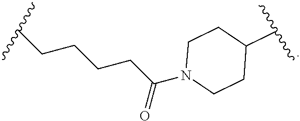 [SMILES: *CCCCC(=O)N1CCC(*)CC1]

In some embodiments, L is

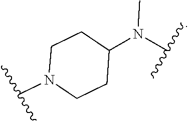 [SMILES: *N1CCC(N(*)C)CC1]

In some embodiments, L is

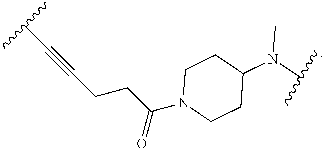 [SMILES: *C#CCCC(=O)N1CCC(N(*)C)CC1]

In some embodiments, L is

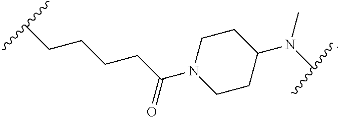 [SMILES: *CCCCC(=O)N1CCC(N(*)C)CC1]

In some embodiments, L is

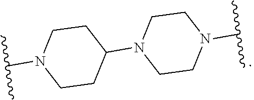 [SMILES: *N1CCC(N2CCN(*)CC2)CC1]

In some embodiments, L is

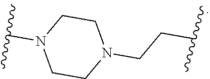 [SMILES: *CCN1CCN(*)CC1]

In some embodiments, L is

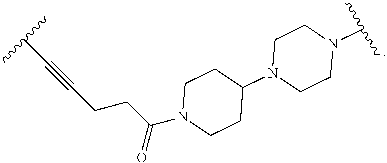 [SMILES: *C#CCCC(=O)N1CCC(N2CCN(*)CC2)CC1]

In some embodiments, L is

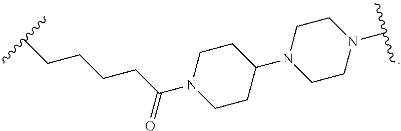 [SMILES: *CCCCC(=O)N1CCC(N2CCN(*)CC2)CC1]

In some embodiments, L is

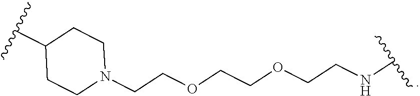 [SMILES: *NCCOCCOCCN1CCC(*)CC1]

In some embodiments, L is

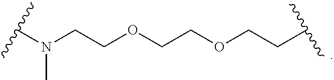 [SMILES: *CCOCCOCCN(*)C]

In some embodiments, L is

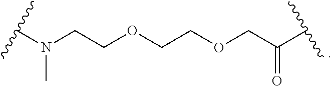 [SMILES: *C(=O)COCCOCCN(*)C]

In some embodiments, L is

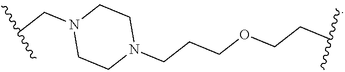 [SMILES: *CCOCCCN1CCN(C*)CC1]

In some embodiments, L is

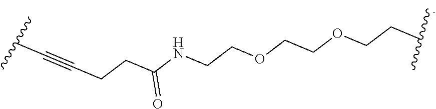 [SMILES: *C#CCCC(=O)NCCOCCOCC*]

In some embodiments, L is

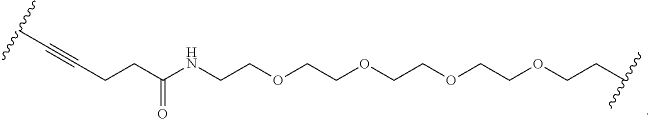 [SMILES: *C#CCCC(=O)NCCOCCOCCOCCOCC*]

In some embodiments, L is

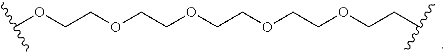 [SMILES: *CCOCCOCCOCCOCCO*]

In some embodiments, L is

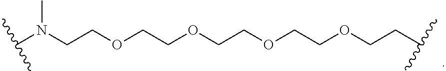 [SMILES: *CCOCCOCCOCCOCCN(*)C]

In some embodiments, L is

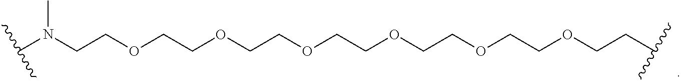 [SMILES: *CCOCCOCCOCCOCCOCCOCCN(*)C]

In some embodiments, L is

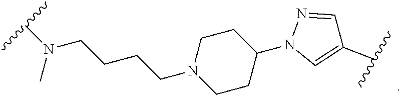 [SMILES: *c1cnn(C2CCN(CCCCN(*)C)CC2)c1]

In some embodiments, L is

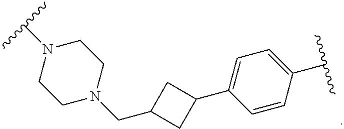 [SMILES: *c1ccc(C2CC(CN3CCN(*)CC3)C2)cc1]

In some embodiments, L is

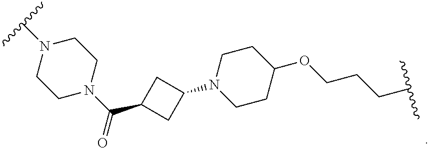 [SMILES: *CCCOC1CCN([C@H]2C[C@H](C(=O)N3CCN(*)CC3)C2)CC1]

In some embodiments, L is

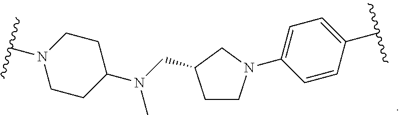 [SMILES: *c1ccc(N2CC[C@H](CN(C)C3CCN(*)CC3)C2)cc1]

In some embodiments, L is

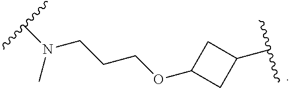 [SMILES: *C1CC(OCCCN(*)C)C1]

In some embodiments, L is

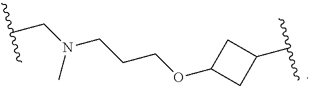 [SMILES: *CN(C)CCCOC1CC(*)C1]

In some embodiments, L is

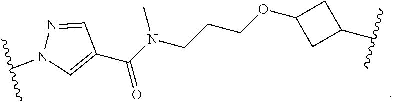 [SMILES: *C1CC(OCCCN(C)C(=O)c2cnn(*)c2)C1]

In some embodiments, L is

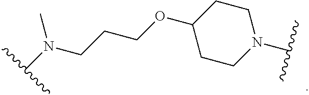 [SMILES: *N(C)CCCOC1CCN(*)CC1]

In some embodiments, L is

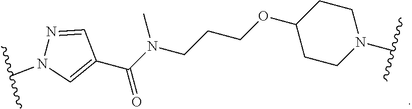 [SMILES: *N1CCC(OCCCN(C)C(=O)c2cnn(*)c2)CC1]

In some embodiments, L is

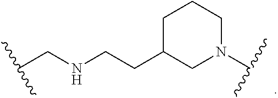 [SMILES: *CNCCC1CCCN(*)C1]

In some embodiments, L is

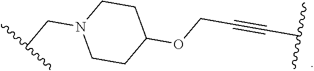 [SMILES: *C#CCOC1CCN(C*)CC1]

In some embodiments, L is

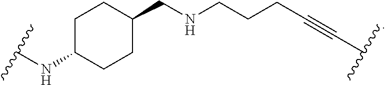 [SMILES: *C#CCCCNC[C@H]1CC[C@H](N*)CC1]

In some embodiments, L is

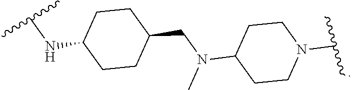 [SMILES: *N[C@H]1CC[C@H](CN(C)C2CCN(*)CC2)CC1]

In some embodiments, L is

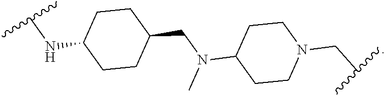 [SMILES: *CN1CCC(N(C)C[C@H]2CC[C@H](N*)CC2)CC1]

In some embodiments, L is

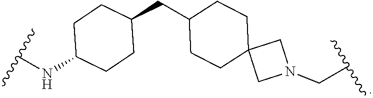 [SMILES: *CN1CC2(CCC(C[C@H]3CC[C@H](N*)CC3)CC2)C1]

In some embodiments, L is

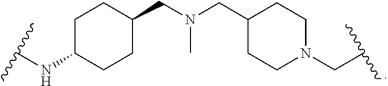 [SMILES: *CN1CCC(CN(C)C[C@H]2CC[C@H](N*)CC2)CC1]

In some embodiments, L is

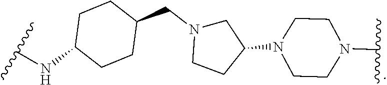 [SMILES: *N[C@H]1CC[C@H](CN2CC[C@@H](N3CCN(*)CC3)C2)CC1]

In some embodiments, L is

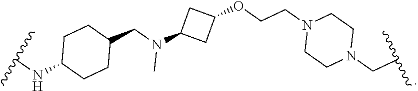

In some embodiments, L is

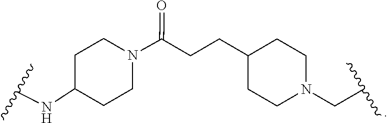 [SMILES: *CN1CCC(CCC(=O)N2CCC(N*)CC2)CC1]

In some embodiments, L is

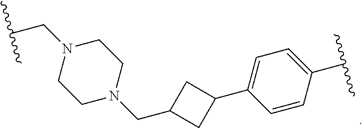 [SMILES: *CN1CCN(CC2CC(c3ccc(*)cc3)C2)CC1]

In some embodiments, L is

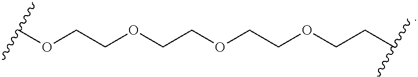 [SMILES: *CCOCCOCCOCCO*]

In some embodiments, L is

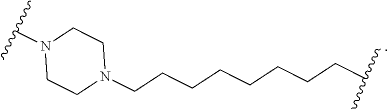 [SMILES: *CCCCCCCCN1CCN(*)CC1]

In some embodiments, L is

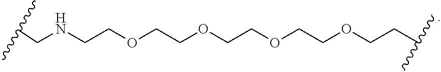 [SMILES: *CCOCCOCCOCCOCCNC*]

In some embodiments, L is

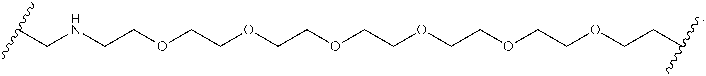 [SMILES: *CCOCCOCCOCCOCCOCCOCCNC*]

In some embodiments, L is

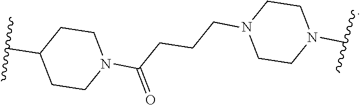 [SMILES: *C1CCN(C(=O)CCCN2CCN(*)CC2)CC1]

In some embodiments, L is

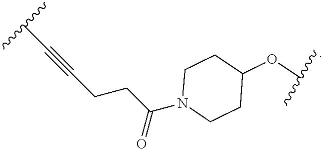 [SMILES: *C#CCCC(=O)N1CCC(O*)CC1]

In some embodiments, L is

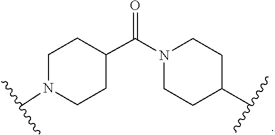 [SMILES: *C1CCN(C(=O)C2CCN(*)CC2)CC1]

In some embodiments, L is

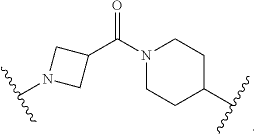 [SMILES: *C1CCN(C(=O)C2CN(*)C2)CC1]

In some embodiments, L is

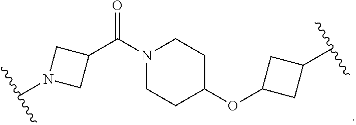 [SMILES: *C1CC(OC2CCN(C(=O)C3CN(*)C3)CC2)C1]

In some embodiments, L is

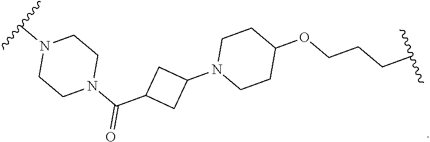 [SMILES: *CCCOC1CCN(C2CC(C(=O)N3CCN(*)CC3)C2)CC1]

In some embodiments, L is

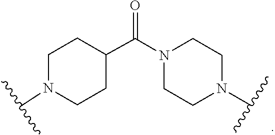 [SMILES: *N1CCC(C(=O)N2CCN(*)CC2)CC1]

In some embodiments L is

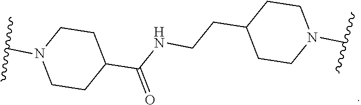 [SMILES: *N1CCC(CCNC(=O)C2CCN(*)CC2)CC1]

In some embodiments, L is

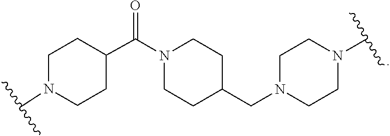 [SMILES: *N1CCC(C(=O)N2CCC(CN3CCN(*)CC3)CC2)CC1]

In some embodiments, L is

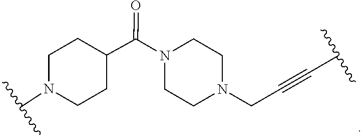 [SMILES: *C#CCN1CCN(C(=O)C2CCN(*)CC2)CC1]

In some embodiments, L is

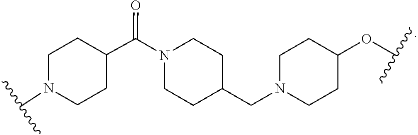 [SMILES: *OC1CCN(CC2CCN(C(=O)C3CCN(*)CC3)CC2)CC1]

In some embodiments, L is

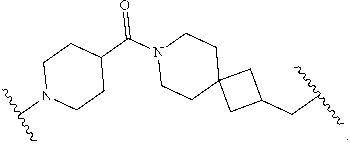 [SMILES: *CC1CC2(CCN(C(=O)C3CCN(*)CC3)CC2)C1]

In some embodiments, L is

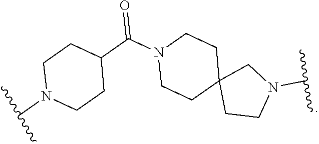 [SMILES: *N1CCC(C(=O)N2CCC3(CCN(*)C3)CC2)CC1]

In some embodiments, L is

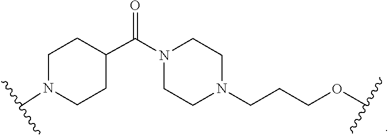 [SMILES: *OCCCN1CCN(C(=O)C2CCN(*)CC2)CC1]

In some embodiments, L is

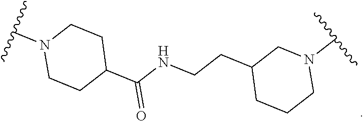 [SMILES: *N1CCC(C(=O)NCCC2CCCN(*)C2)CC1]

In some embodiments, L is

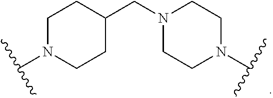 [SMILES: *N1CCC(CN2CCN(*)CC2)CC1]

In some embodiments, L is

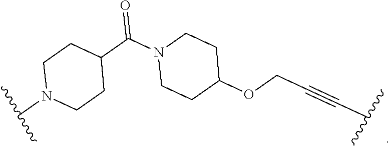 [SMILES: *C#CCOC1CCN(C(=O)C2CCN(*)CC2)CC1]

In some embodiments, L is

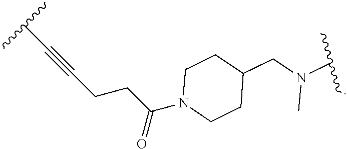 [SMILES: *C#CCCC(=O)N1CCC(CN(*)C)CC1]

In some embodiments, L is

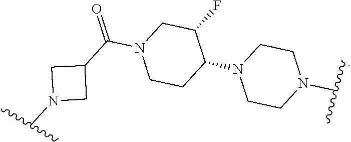 [SMILES: *N1CCN([C@@H]2CCN(C(=O)C3CN(*)C3)C[C@@H]2F)CC1]

In some embodiments, L is

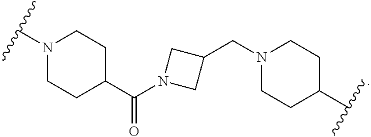 [SMILES: *C1CCN(CC2CN(C(=O)C3CCN(*)CC3)C2)CC1]

In some embodiments, L is

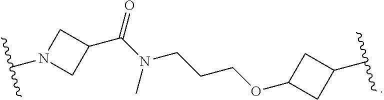 [SMILES: *C1CC(OCCCN(C)C(=O)C2CN(*)C2)C1]

In some embodiments, L is

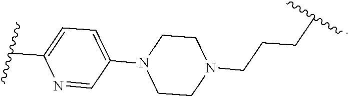 [SMILES: *CCCN1CCN(c2ccc(*)nc2)CC1]

In some embodiments, L is

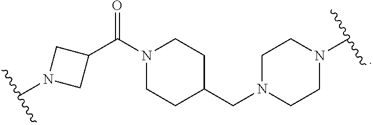 [SMILES: *N1CCN(CC2CCN(C(=O)C3CN(*)C3)CC2)CC1]

In some embodiments, L is

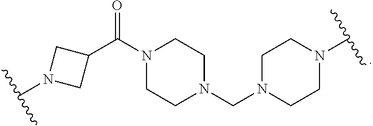 [SMILES: *N1CCN(CN2CCN(C(=O)C3CN(*)C3)CC2)CC1]

In some embodiments, L is

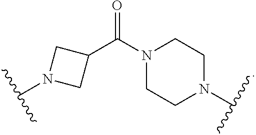 [SMILES: *N1CCN(C(=O)C2CN(*)C2)CC1]

In some embodiments, L is

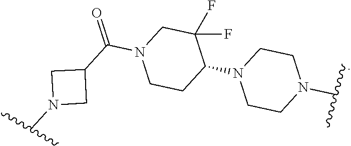 [SMILES: *N1CCN([C@@H]2CCN(C(=O)C3CN(*)C3)CC2(F)F)CC1]

In some embodiments, L is

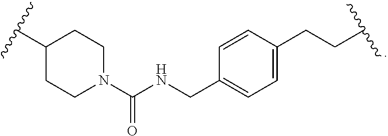 [SMILES: *CCc1ccc(CNC(=O)N2CCC(*)CC2)cc1]

In some embodiments, L is

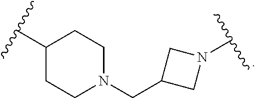 [SMILES: *C1CCN(CC2CN(*)C2)CC1]

In some embodiments, L is

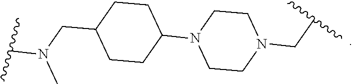 [SMILES: *CN1CCN(C2CCC(CN(*)C)CC2)CC1]

In some embodiments, L is

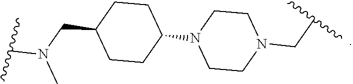 [SMILES: *CN1CCN([C@H]2CC[C@H](CN(*)C)CC2)CC1]

In some embodiments, L is

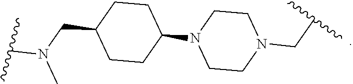 [SMILES: *CN1CCN([C@H]2CC[C@@H](CN(*)C)CC2)CC1]

In some embodiments, L is

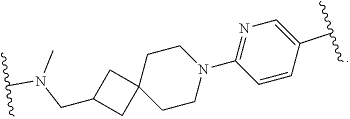 [SMILES: *c1ccc(N2CCC3(CC2)CC(CN(*)C)C3)nc1]

In some embodiments, L is

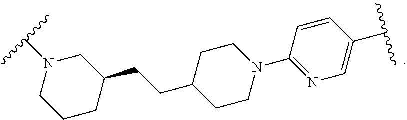 [SMILES: *c1ccc(N2CCC(CC[C@H]3CCCN(*)C3)CC2)nc1]

In some embodiments L is

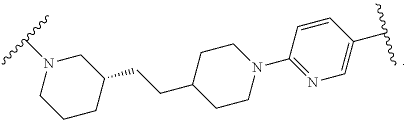 [SMILES: *c1ccc(N2CCC(CC[C@@H]3CCCN(*)C3)CC2)nc1]

In some embodiments, L is

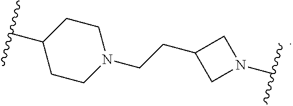 [SMILES: *C1CCN(CCC2CN(*)C2)CC1]

In some embodiments, L is

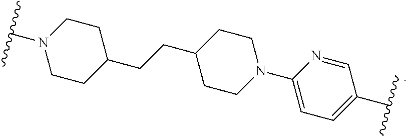 [SMILES: *c1ccc(N2CCC(CCC3CCN(*)CC3)CC2)nc1]

In some embodiments, L is

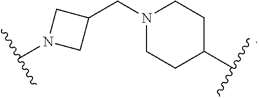 [SMILES: *C1CCN(CC2CN(*)C2)CC1]

In some embodiments, L is

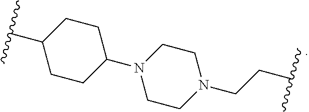 [SMILES: *CCN1CCN(C2CCC(*)CC2)CC1]

In some embodiments, L is

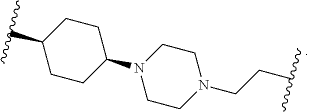 [SMILES: *CCN1CCN([C@H]2CC[C@@H](*)CC2)CC1]

In some embodiments, L is

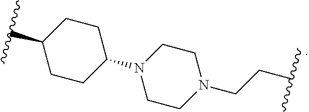 [SMILES: *CCN1CCN([C@H]2CC[C@H](*)CC2)CC1]

In some embodiments, L is

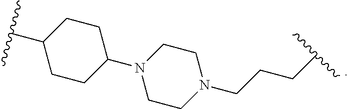 [SMILES: *CCCN1CCN(C2CCC(*)CC2)CC1]

In some embodiments, L is

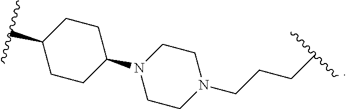 [SMILES: *CCCN1CCN([C@H]2CC[C@@H](*)CC2)CC1]

In some embodiments, L is

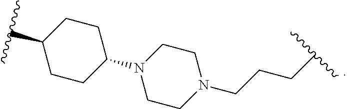 [SMILES: *CCCN1CCN([C@H]2CC[C@H](*)CC2)CC1]

In some embodiments, L is

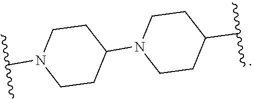 [SMILES: *C1CCN(C2CCN(*)CC2)CC1]

In some embodiments, L is

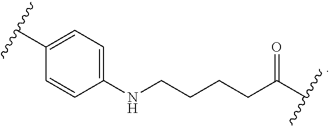 [SMILES: *C(=O)CCCCNc1ccc(*)cc1]

In some embodiments, L is

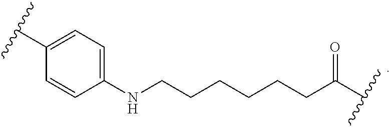 [SMILES: *C(=O)CCCCCCNc1ccc(*)cc1]

In some embodiments, L is

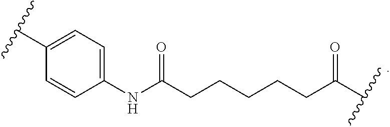 [SMILES: *C(=O)CCCCCC(=O)Nc1ccc(*)cc1]

In some embodiments, L is

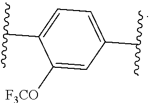 [SMILES: *c1ccc(*)c(OC(F)(F)F)c1]

In some embodiments, L is

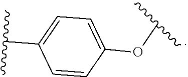 [SMILES: *Oc1ccc(*)cc1]

In some embodiments, L is

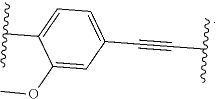 [SMILES: *C#Cc1ccc(*)c(OC)c1]

In some embodiments, L is

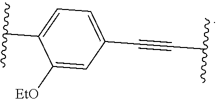 [SMILES: *C#Cc1ccc(*)c(OCC)c1]

In some embodiments, L is

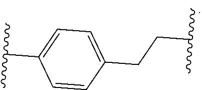 [SMILES: *CCc1ccc(*)cc1]

In some embodiments, L is

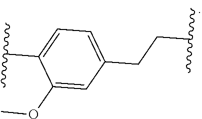 [SMILES: *CCc1ccc(*)c(OC)c1]

In some embodiments, L is

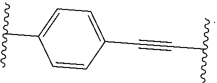 [SMILES: *C#Cc1ccc(*)cc1]

In some embodiments, L is

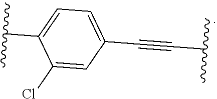 [SMILES: *C#Cc1ccc(*)c(Cl)c1]

In some embodiments, L is

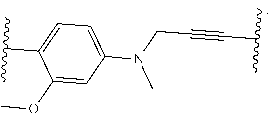 [SMILES: *C#CCN(C)c1ccc(*)c(OC)c1]

In some embodiments, L is

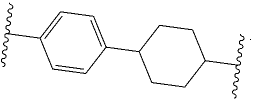 [SMILES: *c1ccc(C2CCC(*)CC2)cc1]

In some embodiments, L is

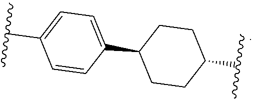 [SMILES: *c1ccc([C@H]2CC[C@H](*)CC2)cc1]

In some embodiments, L is

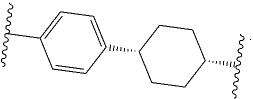 [SMILES: *c1ccc([C@H]2CC[C@@H](*)CC2)cc1]

In some embodiments, L is

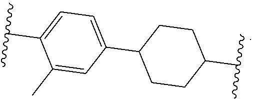 [SMILES: *c1ccc(C2CCC(*)CC2)cc1C]

In some embodiments, L is

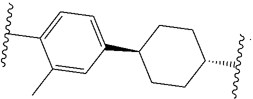 [SMILES: *c1ccc([C@H]2CC[C@H](*)CC2)cc1C]

In some embodiments, L is

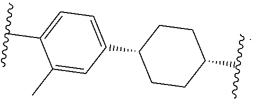 [SMILES: *c1ccc([C@H]2CC[C@@H](*)CC2)cc1C]

In some embodiments, L is

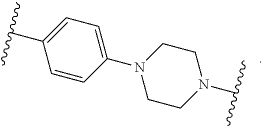 [SMILES: *c1ccc(N2CCN(*)CC2)cc1]

In some embodiments, L is

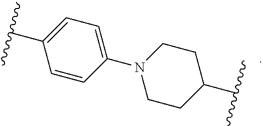 [SMILES: *c1ccc(N2CCC(*)CC2)cc1]

In some embodiments, L is

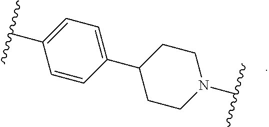 [SMILES: *c1ccc(C2CCN(*)CC2)cc1]

In some embodiments, L is

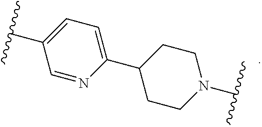 [SMILES: *c1ccc(C2CCN(*)CC2)nc1]

In some embodiments, L is

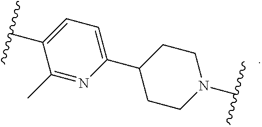 [SMILES: *c1ccc(C2CCN(*)CC2)nc1C]

In some embodiments, L is

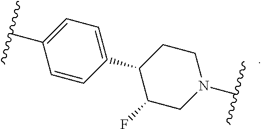 [SMILES: *c1ccc([C@@H]2CCN(*)C[C@@H]2F)cc1]

In some embodiments, L is

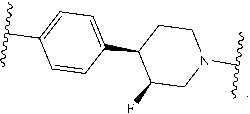 [SMILES: *c1ccc([C@H]2CCN(*)C[C@H]2F)cc1]

In some embodiments, L is

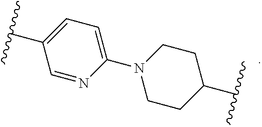 [SMILES: *c1ccc(N2CCC(*)CC2)nc1]

In some embodiments, L is

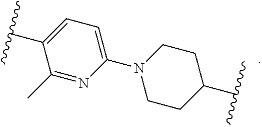 [SMILES: *c1ccc(N2CCC(*)CC2)nc1C]

In some embodiments, L is

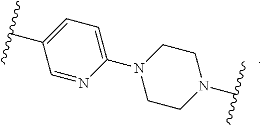 [SMILES: *c1ccc(N2CCN(*)CC2)nc1]

In some embodiments, L is

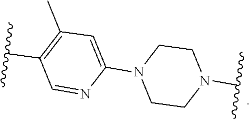 [SMILES: *c1cnc(N2CCN(*)CC2)cc1C]

In some embodiments, L is

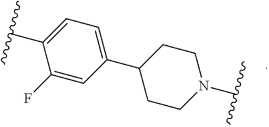 [SMILES: *c1ccc(C2CCN(*)CC2)cc1F]

In some embodiments, L is

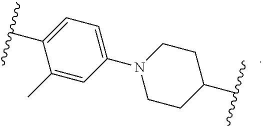 [SMILES: *c1ccc(N2CCC(*)CC2)cc1C]

In some embodiments, L is

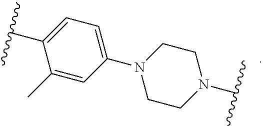 [SMILES: *c1ccc(N2CCN(*)CC2)cc1C]

In some embodiments, L is

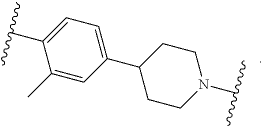 [SMILES: *c1ccc(C2CCN(*)CC2)cc1C]

In some embodiments, L is

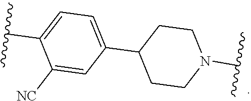 [SMILES: *c1ccc(C2CCN(*)CC2)cc1C#N]

In some embodiments, L is

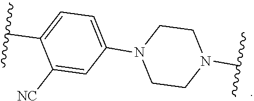 [SMILES: *c1ccc(N2CCN(*)CC2)cc1C#N]

In some embodiments, L is

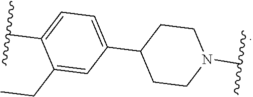 [SMILES: *c1ccc(C2CCN(*)CC2)cc1CC]

In some embodiments, L is

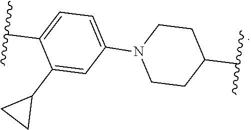 [SMILES: *c1ccc(N2CCC(*)CC2)cc1C1CC1]

In some embodiments, L is

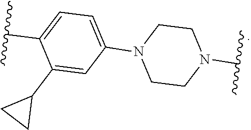 [SMILES: *c1ccc(N2CCN(*)CC2)cc1C1CC1]

In some embodiments, L is

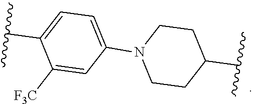 [SMILES: *c1ccc(N2CCC(*)CC2)cc1C(F)(F)F]

In some embodiments, L is F

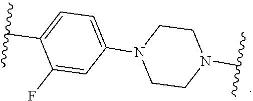 [SMILES: *c1ccc(N2CCN(*)CC2)cc1F]

In some embodiments, L is

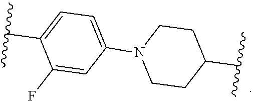 [SMILES: *c1ccc(N2CCC(*)CC2)cc1F]

In some embodiments, L is

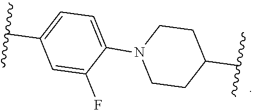 [SMILES: *c1ccc(N2CCC(*)CC2)c(F)c1]

In some embodiments, L is

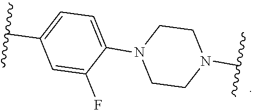 [SMILES: *c1ccc(N2CCN(*)CC2)c(F)c1]

In some embodiments, L is

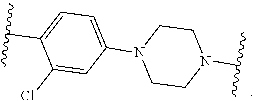 [SMILES: *c1ccc(N2CCN(*)CC2)cc1Cl]

In some embodiments, L is

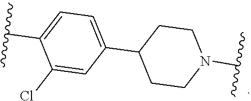 [SMILES: *c1ccc(C2CCN(*)CC2)cc1Cl]

In some embodiments, L is

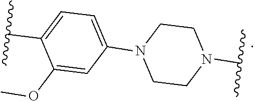 [SMILES: *c1ccc(N2CCN(*)CC2)cc1OC]

In some embodiments, L is

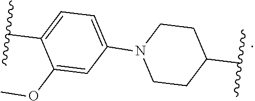 [SMILES: *c1ccc(N2CCC(*)CC2)cc1OC]

In some embodiments, L is

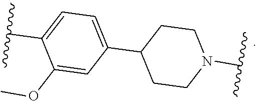 [SMILES: *c1ccc(C2CCN(*)CC2)cc1OC]

In some embodiments, L is

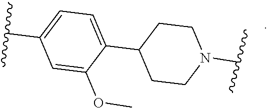 [SMILES: *c1ccc(C2CCN(*)CC2)c(OC)c1]

In some embodiments, L is

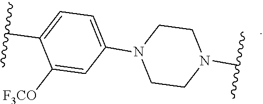 [SMILES: *c1ccc(N2CCN(*)CC2)cc1OC(F)(F)F]

In some embodiments, L is

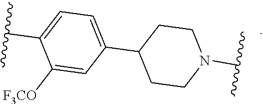 [SMILES: *c1ccc(C2CCN(*)CC2)cc1OC(F)(F)F]

In some embodiments, L is

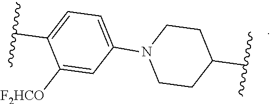 [SMILES: *c1ccc(N2CCC(*)CC2)cc1OC(F)F]

In some embodiments, L is

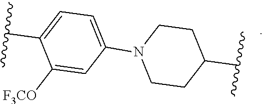 [SMILES: *c1ccc(N2CCC(*)CC2)cc1OC(F)(F)F]

In some embodiments, L is

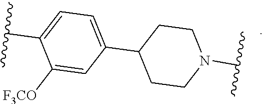 [SMILES: *c1ccc(C2CCN(*)CC2)cc1OC(F)(F)F]

In some embodiments, L is

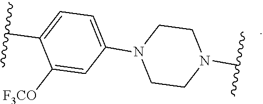 [SMILES: *c1ccc(N2CCN(*)CC2)cc1OC(F)(F)F]

In some embodiments, L is

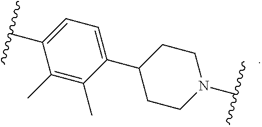 [SMILES: *c1ccc(C2CCN(*)CC2)c(C)c1C]

In some embodiments, L is

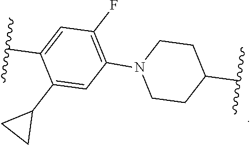 [SMILES: *c1cc(F)c(N2CCC(*)CC2)cc1C1CC1]

In some embodiments, L is

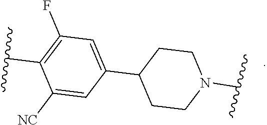 [SMILES: *c1c(F)cc(C2CCN(*)CC2)cc1C#N]

In some embodiments L is

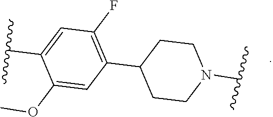 [SMILES: *c1cc(F)c(C2CCN(*)CC2)cc1OC]

In some embodiments, L is

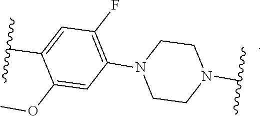 [SMILES: *c1cc(F)c(N2CCN(*)CC2)cc1OC]

In some embodiments, L is

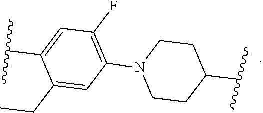 [SMILES: *c1cc(F)c(N2CCC(*)CC2)cc1CC]

In some embodiments, L is O

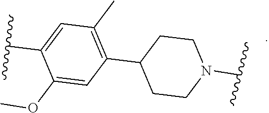 [SMILES: *c1cc(C)c(C2CCN(*)CC2)cc1OC]

In some embodiments, L is

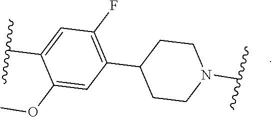 [SMILES: *c1cc(F)c(C2CCN(*)CC2)cc1OC]

In some embodiments, L is

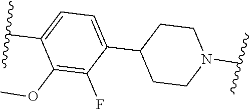 [SMILES: *c1ccc(C2CCN(*)CC2)c(F)c1OC]

In some embodiments, L is

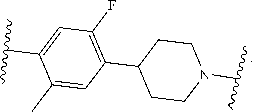 [SMILES: *c1cc(F)c(C2CCN(*)CC2)cc1C]

In some embodiments, L is

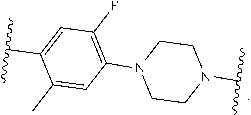 [SMILES: *c1cc(F)c(N2CCN(*)CC2)cc1C]

In some embodiments, L is

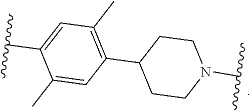 [SMILES: *c1cc(C)c(C2CCN(*)CC2)cc1C]

In some embodiments, L is

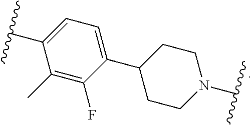 [SMILES: *c1ccc(C2CCN(*)CC2)c(F)c1C]

In some embodiments, L is

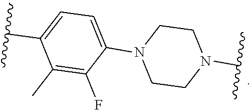 [SMILES: *c1ccc(N2CCN(*)CC2)c(F)c1C]

In some embodiments, L is

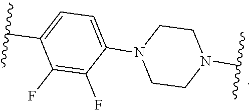 [SMILES: *c1ccc(N2CCN(*)CC2)c(F)c1F]

In some embodiments, L is

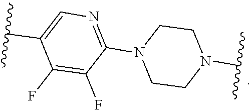 [SMILES: *c1cnc(N2CCN(*)CC2)c(F)c1F]

In some embodiments, L is

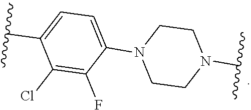 [SMILES: *c1ccc(N2CCN(*)CC2)c(F)c1Cl]

In some embodiments, L is

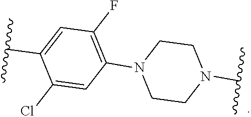 [SMILES: *c1cc(F)c(N2CCN(*)CC2)cc1Cl]

In some embodiments, L is

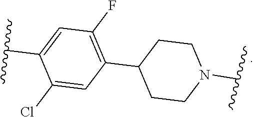 [SMILES: *c1cc(F)c(C2CCN(*)CC2)cc1Cl]

In some embodiments, L is

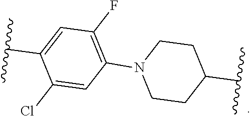 [SMILES: *c1cc(F)c(N2CCC(*)CC2)cc1Cl]

In some embodiments, L is

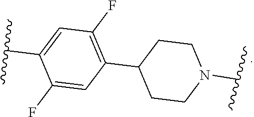 [SMILES: *c1cc(F)c(C2CCN(*)CC2)cc1F]

In some embodiments, L is

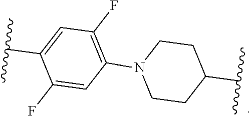 [SMILES: *c1cc(F)c(N2CCC(*)CC2)cc1F]

In some embodiments, L is

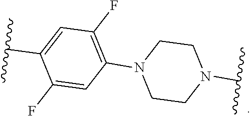 [SMILES: *c1cc(F)c(N2CCN(*)CC2)cc1F]

In some embodiments, L is

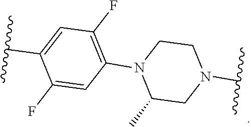 [SMILES: *c1cc(F)c(N2CCN(*)C[C@@H]2C)cc1F]

In some embodiments, L is

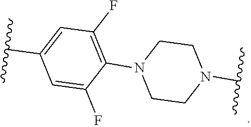 [SMILES: *c1cc(F)c(N2CCN(*)CC2)c(F)c1]

In some embodiments, L is

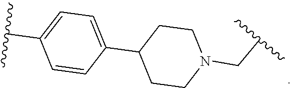 [SMILES: *CN1CCC(c2ccc(*)cc2)CC1]

In some embodiments, L is

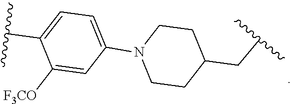 [SMILES: *CC1CCN(c2ccc(*)c(OC(F)(F)F)c2)CC1]

In some embodiments, L is

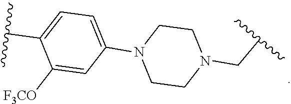 [SMILES: *CN1CCN(c2ccc(*)c(OC(F)(F)F)c2)CC1]

In some embodiments, L is

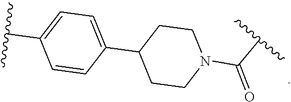 [SMILES: *C(=O)N1CCC(c2ccc(*)cc2)CC1]

In some embodiments, L is

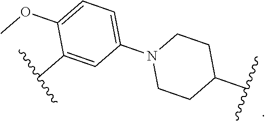 [SMILES: *c1cc(N2CCC(*)CC2)ccc1OC]

In some embodiments, L is

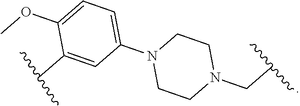 [SMILES: *CN1CCN(c2ccc(OC)c(*)c2)CC1]

In some embodiments, L is

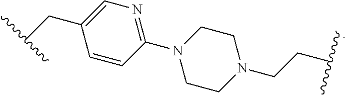 [SMILES: *CCN1CCN(c2ccc(C*)cn2)CC1]

In some embodiments, L is

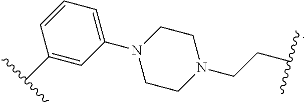 [SMILES: *CCN1CCN(c2cccc(*)c2)CC1]

In some embodiments, L is

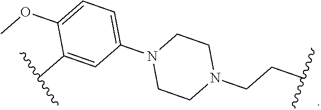 [SMILES: *CCN1CCN(c2ccc(OC)c(*)c2)CC1]

In some embodiments, L is

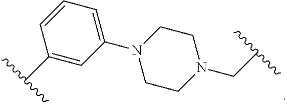 [SMILES: *CN1CCN(c2cccc(*)c2)CC1]

In some embodiments, L is

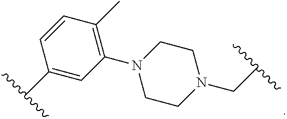 [SMILES: *CN1CCN(c2cc(*)ccc2C)CC1]

In some embodiments, L is

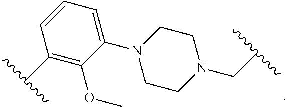 [SMILES: *CN1CCN(c2cccc(*)c2OC)CC1]

In some embodiments, L is

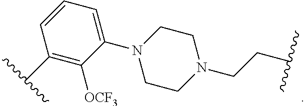 [SMILES: *CCN1CCN(c2cccc(*)c2OC(F)(F)F)CC1]

In some embodiments, L is

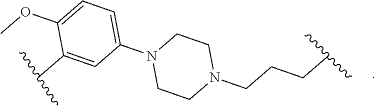 [SMILES: *CCCN1CCN(c2ccc(OC)c(*)c2)CC1]

In some embodiments, L is

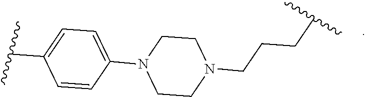 [SMILES: *CCCN1CCN(c2ccc(*)cc2)CC1]

In some embodiments, L is

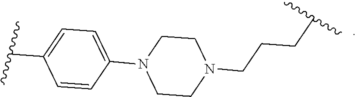 [SMILES: *CCCN1CCN(c2ccc(*)cc2)CC1]

In some embodiments, L is

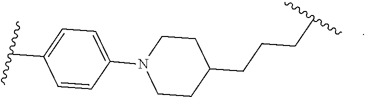 [SMILES: *CCCC1CCN(c2ccc(*)cc2)CC1]

In some embodiments, L is

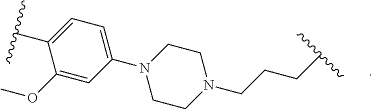 [SMILES: *CCCN1CCN(c2ccc(*)c(OC)c2)CC1]

In some embodiments, L is

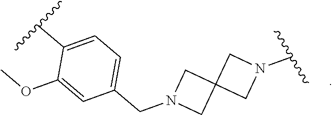 [SMILES: *c1ccc(CN2CC3(CN(*)C3)C2)cc1OC]

In some embodiments, L is

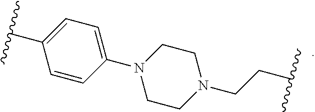 [SMILES: *CCN1CCN(c2ccc(*)cc2)CC1]

In some embodiments, L is

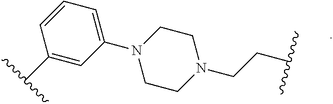 [SMILES: *CCN1CCN(c2cccc(*)c2)CC1]

In some embodiments, L is

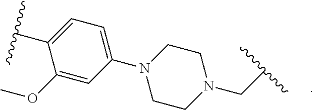 [SMILES: *CN1CCN(c2ccc(*)c(OC)c2)CC1]

In some embodiments, L is

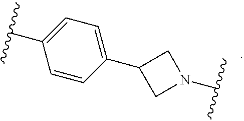 [SMILES: *c1ccc(C2CN(*)C2)cc1]

In some embodiments, L is

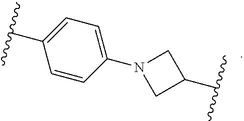 [SMILES: *c1ccc(N2CC(*)C2)cc1]

In some embodiments, L is

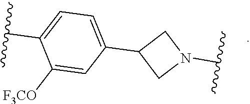 [SMILES: *c1ccc(C2CN(*)C2)cc1OC(F)(F)F]

In some embodiments, L is

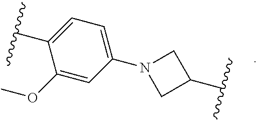 [SMILES: *c1ccc(N2CC(*)C2)cc1OC]

In some embodiments, L is

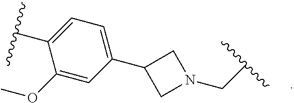 [SMILES: *CN1CC(c2ccc(*)c(OC)c2)C1]

In some embodiments, L is

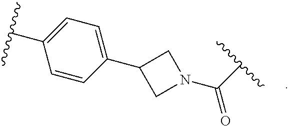 [SMILES: *C(=O)N1CC(c2ccc(*)cc2)C1]

In some embodiments, L is

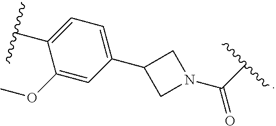 [SMILES: *C(=O)N1CC(c2ccc(*)c(OC)c2)C1]

In some embodiments, L is

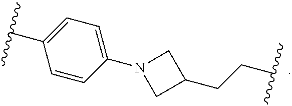 [SMILES: *CCC1CN(c2ccc(*)cc2)C1]

In some embodiments, L is

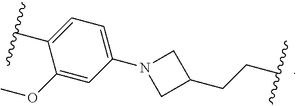 [SMILES: *CCC1CN(c2ccc(*)c(OC)c2)C1]

In some embodiments, L is

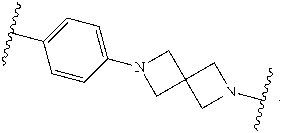 [SMILES: *c1ccc(N2CC3(CN(*)C3)C2)cc1]

In some embodiments, L is

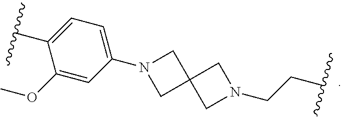 [SMILES: *CCN1CC2(C1)CN(c1ccc(*)c(OC)c1)C2]

In some embodiments, L is

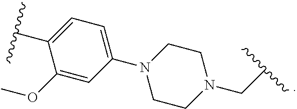 [SMILES: *CN1CCN(c2ccc(*)c(OC)c2)CC1]

In some embodiments, L is

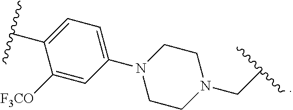 [SMILES: *CN1CCN(c2ccc(*)c(OC(F)(F)F)c2)CC1]

In some embodiments, L is

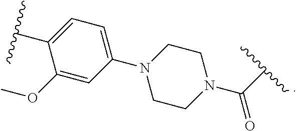 [SMILES: *C(=O)N1CCN(c2ccc(*)c(OC)c2)CC1]

In some embodiments, L is

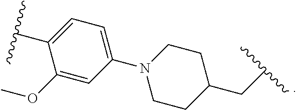 [SMILES: *CC1CCN(c2ccc(*)c(OC)c2)CC1]

In some embodiments, L is

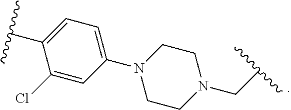 [SMILES: *CN1CCN(c2ccc(*)c(Cl)c2)CC1]

In some embodiments, L is

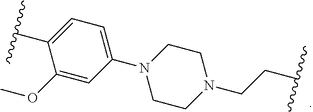 [SMILES: *CCN1CCN(c2ccc(*)c(OC)c2)CC1]

In some embodiments, L is

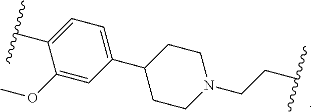 [SMILES: *CCN1CCC(c2ccc(*)c(OC)c2)CC1]

In some embodiments, L is

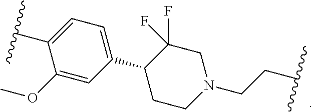 [SMILES: *CCN1CC[C@H](c2ccc(*)c(OC)c2)C(F)(F)C1]

In some embodiments, L is

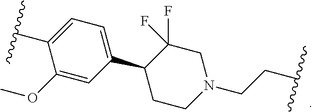 [SMILES: *CCN1CC[C@@H](c2ccc(*)c(OC)c2)C(F)(F)C1]

In some embodiments, L is

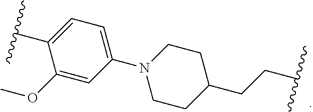 [SMILES: *CCC1CCN(c2ccc(*)c(OC)c2)CC1]

In some embodiments, L is

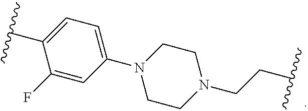 [SMILES: *CCN1CCN(c2ccc(*)c(F)c2)CC1]

In some embodiments, L is

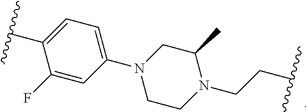 [SMILES: *CCN1CCN(c2ccc(*)c(F)c2)C[C@H]1C]

In some embodiments, L is

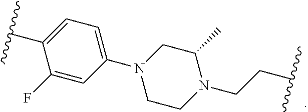 [SMILES: *CCN1CCN(c2ccc(*)c(F)c2)C[C@@H]1C]

In some embodiments, L is

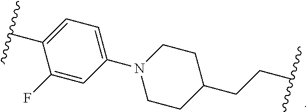 [SMILES: *CCC1CCN(c2ccc(*)c(F)c2)CC1]

In some embodiments, L is

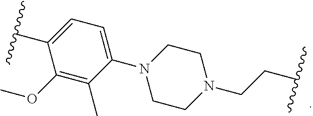 [SMILES: *CCN1CCN(c2ccc(*)c(OC)c2C)CC1]

In some embodiments, L is

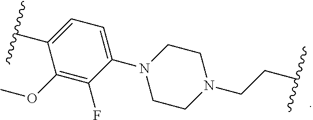 [SMILES: *CCN1CCN(c2ccc(*)c(OC)c2F)CC1]

In some embodiments, L is

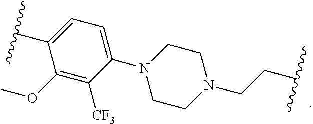 [SMILES: *CCN1CCN(c2ccc(*)c(OC)c2C(F)(F)F)CC1]

In some embodiments, L is

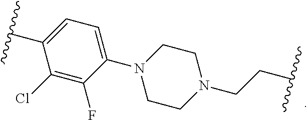 [SMILES: *CCN1CCN(c2ccc(*)c(Cl)c2F)CC1]

In some embodiments, L is

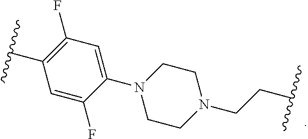 [SMILES: *CCN1CCN(c2cc(F)c(*)cc2F)CC1]

In some embodiments, L is

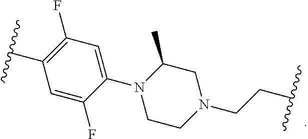 [SMILES: *CCN1CCN(c2cc(F)c(*)cc2F)[C@@H](C)C1]

In some embodiments, L is

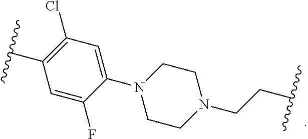 [SMILES: *CCN1CCN(c2cc(Cl)c(*)cc2F)CC1]

In some embodiments, L is

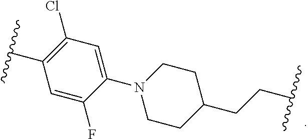 [SMILES: *CCC1CCN(c2cc(Cl)c(*)cc2F)CC1]

In some embodiments, L is

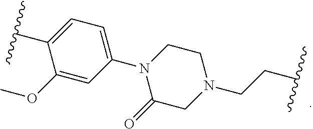 [SMILES: *CCN1CCN(c2ccc(*)c(OC)c2)C(=O)C1]

In some embodiments, L is

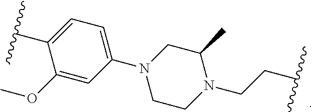 [SMILES: *CCN1CCN(c2ccc(*)c(OC)c2)C[C@H]1C]

In some embodiments, L is

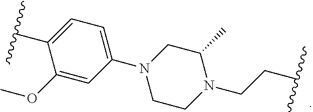 [SMILES: *CCN1CCN(c2ccc(*)c(OC)c2)C[C@@H]1C]

In some embodiments, L is

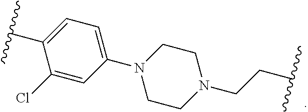 [SMILES: *CCN1CCN(c2ccc(*)c(Cl)c2)CC1]

In some embodiments, L is

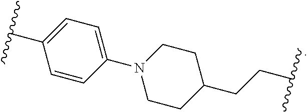 [SMILES: *CCC1CCN(c2ccc(*)cc2)CC1]

In some embodiments L is

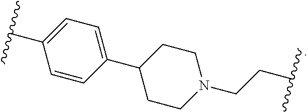 [SMILES: *CCN1CCC(c2ccc(*)cc2)CC1]

In some embodiments, L is

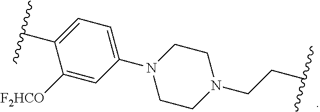 [SMILES: *CCN1CCN(c2ccc(*)c(OC(F)F)c2)CC1]

In some embodiments, L is

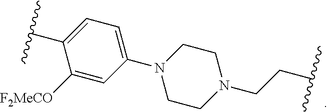

In some embodiments, L is

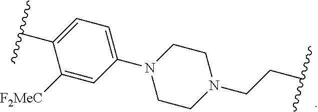

In some embodiments, L is

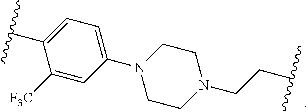 [SMILES: *CCN1CCN(c2ccc(*)c(C(F)(F)F)c2)CC1]

In some embodiments, L is

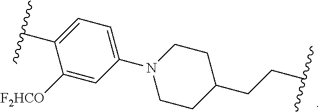 [SMILES: *CCC1CCN(c2ccc(*)c(OC(F)F)c2)CC1]

In some embodiments, L is

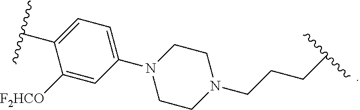 [SMILES: *CCCN1CCN(c2ccc(*)c(OC(F)F)c2)CC1]

In some embodiments, L is

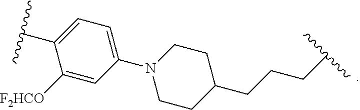 [SMILES: *CCCC1CCN(c2ccc(*)c(OC(F)F)c2)CC1]

In some embodiments, L is

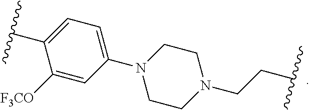 [SMILES: *CCN1CCN(c2ccc(*)c(OC(F)(F)F)c2)CC1]

In some embodiments, L is

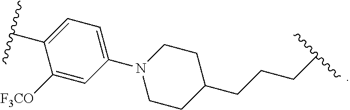 [SMILES: *CCCC1CCN(c2ccc(*)c(OC(F)(F)F)c2)CC1]

In some embodiments, L is

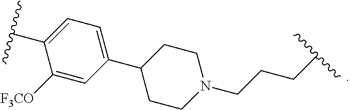 [SMILES: *CCCN1CCC(c2ccc(*)c(OC(F)(F)F)c2)CC1]

In some embodiments, L is

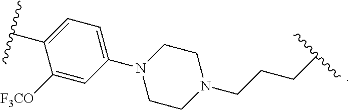 [SMILES: *CCCN1CCN(c2ccc(*)c(OC(F)(F)F)c2)CC1]

In some embodiments, L is

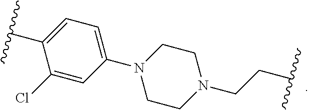 [SMILES: *CCN1CCN(c2ccc(*)c(Cl)c2)CC1]

In some embodiments, L is

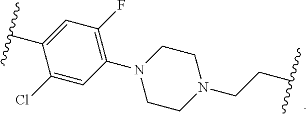 [SMILES: *CCN1CCN(c2cc(Cl)c(*)cc2F)CC1]

In some embodiments, L is

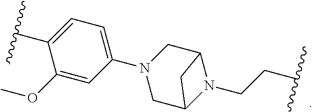 [SMILES: *CCN1C2CC1CN(c1ccc(*)c(OC)c1)C2]

In some embodiments, L is

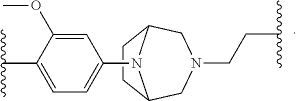 [SMILES: *CCN1CC2CCC(C1)N2c1ccc(*)c(OC)c1]

In some embodiments, L is

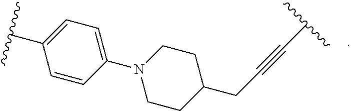 [SMILES: *C#CCC1CCN(c2ccc(*)cc2)CC1]

In some embodiments, L is

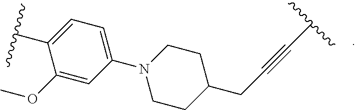 [SMILES: *C#CCC1CCN(c2ccc(*)c(OC)c2)CC1]

In some embodiments, L is

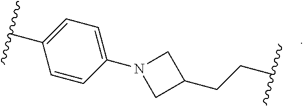 [SMILES: *CCC1CN(c2ccc(*)cc2)C1]

In some embodiments, L is

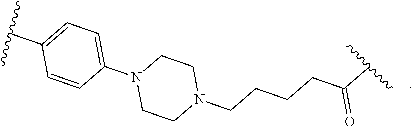 [SMILES: *C(=O)CCCCN1CCN(c2ccc(*)cc2)CC1]

In some embodiments, L is

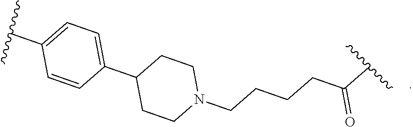 [SMILES: *C(=O)CCCCN1CCC(c2ccc(*)cc2)CC1]

In some embodiments, L is

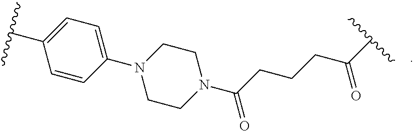 [SMILES: *C(=O)CCCC(=O)N1CCN(c2ccc(*)cc2)CC1]

In some embodiments, L is

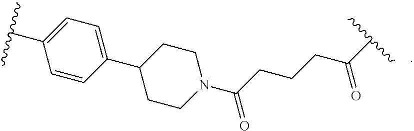 [SMILES: *C(=O)CCCC(=O)N1CCC(c2ccc(*)cc2)CC1]

In some embodiments, L is

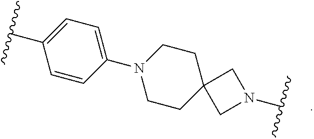 [SMILES: *c1ccc(N2CCC3(CC2)CN(*)C3)cc1]

In some embodiments, L is

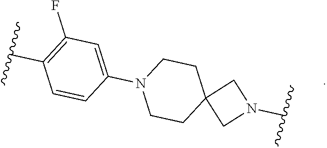 [SMILES: *c1ccc(N2CCC3(CC2)CN(*)C3)cc1F]

In some embodiments, L is

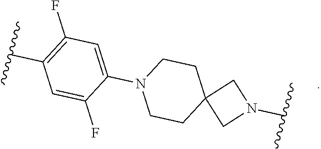 [SMILES: *c1cc(F)c(N2CCC3(CC2)CN(*)C3)cc1F]

In some embodiments, L is

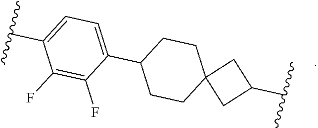 [SMILES: *c1ccc(C2CCC3(CC2)CC(*)C3)c(F)c1F]

In some embodiments, L is

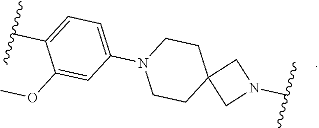 [SMILES: *c1ccc(N2CCC3(CC2)CN(*)C3)cc1OC]

In some embodiments, L is

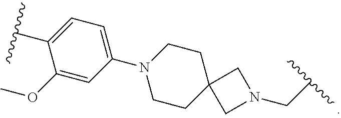 [SMILES: *CN1CC2(CCN(c3ccc(*)c(OC)c3)CC2)C1]

In some embodiments, L is

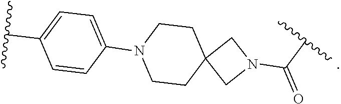 [SMILES: *C(=O)N1CC2(CCN(c3ccc(*)cc3)CC2)C1]

In some embodiments, L is

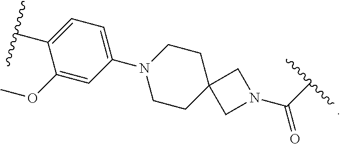 [SMILES: *C(=O)N1CC2(CCN(c3ccc(*)c(OC)c3)CC2)C1]

In some embodiments, L is

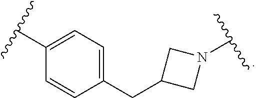 [SMILES: *c1ccc(CC2CN(*)C2)cc1]

In some embodiments, L is

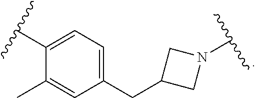 [SMILES: *c1ccc(CC2CN(*)C2)cc1C]

In some embodiments, L is

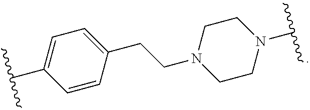 [SMILES: *c1ccc(CCN2CCN(*)CC2)cc1]

In some embodiments, L is

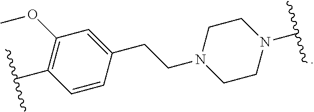 [SMILES: *c1ccc(CCN2CCN(*)CC2)cc1OC]

In some embodiments, L is

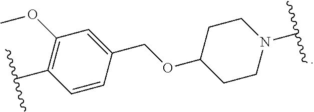 [SMILES: *c1ccc(COC2CCN(*)CC2)cc1OC]

In some embodiments, L is

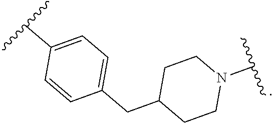 [SMILES: *c1ccc(CC2CCN(*)CC2)cc1]

In some embodiments, L is

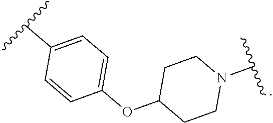 [SMILES: *c1ccc(OC2CCN(*)CC2)cc1]

In some embodiments, L is

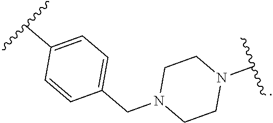 [SMILES: *c1ccc(CN2CCN(*)CC2)cc1]

In some embodiments, L is

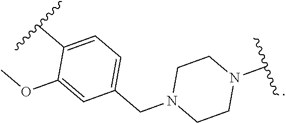 [SMILES: *c1ccc(CN2CCN(*)CC2)cc1OC]

In some embodiments, L is

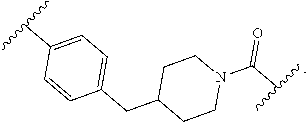 [SMILES: *C(=O)N1CCC(Cc2ccc(*)cc2)CC1]

In some embodiments, L is

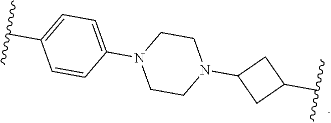 [SMILES: *c1ccc(N2CCN(C3CC(*)C3)CC2)cc1]

In some embodiments, L is

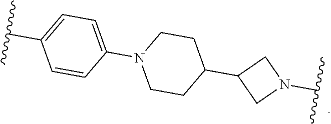 [SMILES: *c1ccc(N2CCC(C3CN(*)C3)CC2)cc1]

In some embodiments, L is

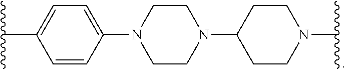 [SMILES: *c1ccc(N2CCN(C3CCN(*)CC3)CC2)cc1]

In some embodiments, L is

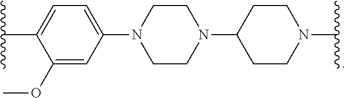 [SMILES: *c1ccc(N2CCN(C3CCN(*)CC3)CC2)cc1OC]

In some embodiments, L is

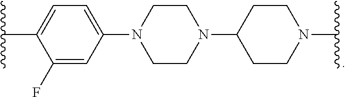 [SMILES: *c1ccc(N2CCN(C3CCN(*)CC3)CC2)cc1F]

In some embodiments, L is

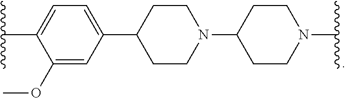 [SMILES: *c1ccc(C2CCN(C3CCN(*)CC3)CC2)cc1OC]

In some embodiments, L is

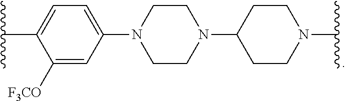 [SMILES: *c1ccc(N2CCN(C3CCN(*)CC3)CC2)cc1OC(F)(F)F]

In some embodiments, L is

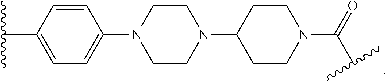 [SMILES: *C(=O)N1CCC(N2CCN(c3ccc(*)cc3)CC2)CC1]

In some embodiments, L is

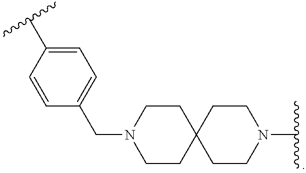 [SMILES: *c1ccc(CN2CCC3(CCN(*)CC3)CC2)cc1]

In some embodiments, L is

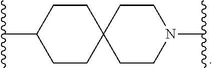 [SMILES: *C1CCC2(CC1)CCN(*)CC2]

In some embodiments, L is

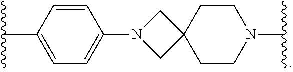 [SMILES: *c1ccc(N2CC3(CCN(*)CC3)C2)cc1]

In some embodiments, L is

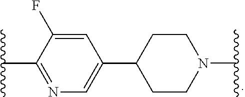 [SMILES: *c1ncc(C2CCN(*)CC2)cc1F]

In some embodiments, L is

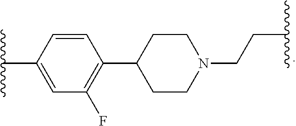 [SMILES: *CCN1CCC(c2ccc(*)cc2F)CC1]

In some embodiments, L is

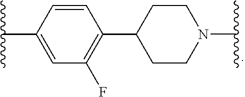 [SMILES: *c1ccc(C2CCN(*)CC2)c(F)c1]

In some embodiments, L is

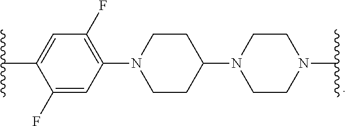 [SMILES: *c1cc(F)c(N2CCC(N3CCN(*)CC3)CC2)cc1F]

In some embodiments, L is

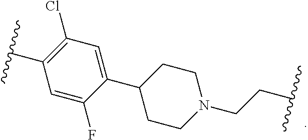 [SMILES: *CCN1CCC(c2cc(Cl)c(*)cc2F)CC1]

In some embodiments, L is

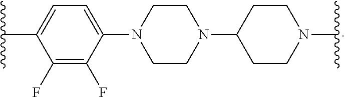 [SMILES: *c1ccc(N2CCN(C3CCN(*)CC3)CC2)c(F)c1F]

In some embodiments, L is

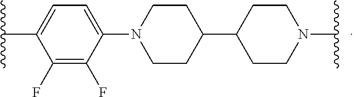 [SMILES: *c1ccc(N2CCC(C3CCN(*)CC3)CC2)c(F)c1F]

In some embodiments, L is

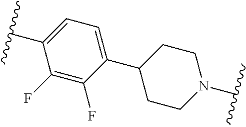 [SMILES: *c1ccc(C2CCN(*)CC2)c(F)c1F]

In some embodiments, L is

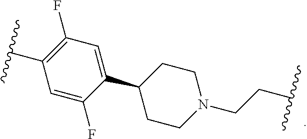

In some embodiments, L is

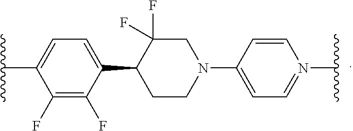

In some embodiments, L is

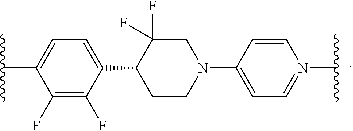

In some embodiments, L is

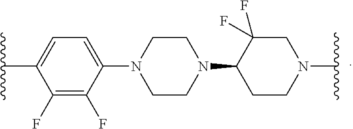 [SMILES: *c1ccc(N2CCN([C@@H]3CCN(*)CC3(F)F)CC2)c(F)c1F]

In some embodiments, L is

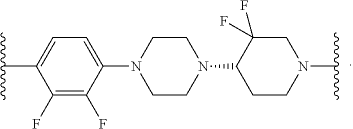

In some embodiments, L is

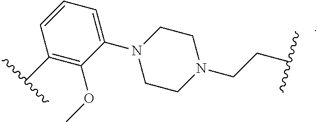 [SMILES: *CCN1CCN(c2cccc(*)c2OC)CC1]

In some embodiments, L is

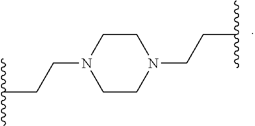 [SMILES: *CCN1CCN(CC*)CC1]

In some embodiments, L is

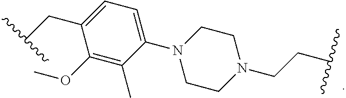 [SMILES: *CCN1CCN(c2ccc(C*)c(OC)c2C)CC1]

In some embodiments, L is

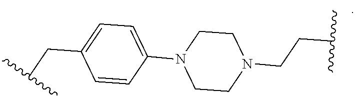 [SMILES: *CCN1CCN(c2ccc(C*)cc2)CC1]

In some embodiments, L is

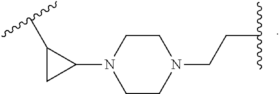 [SMILES: *CCN1CCN(C2CC2*)CC1]

In some embodiments, L is

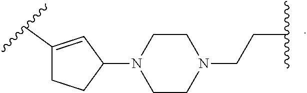 [SMILES: *CCN1CCN(C2C=C(*)CC2)CC1]

In some embodiments, L is

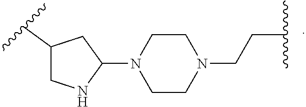 [SMILES: *CCN1CCN(C2CC(*)CN2)CC1]

In some embodiments, L is

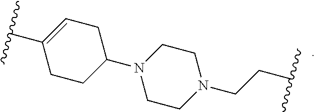 [SMILES: *CCN1CCN(C2CC=C(*)CC2)CC1]

In some embodiments, L is

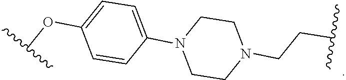 [SMILES: *CCN1CCN(c2ccc(O*)cc2)CC1]

In some embodiments, L is

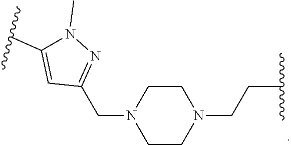 [SMILES: *CCN1CCN(Cc2cc(*)n(C)n2)CC1]

In some embodiments, L is

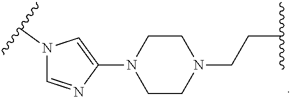 [SMILES: *CCN1CCN(c2cn(*)cn2)CC1]

In some embodiments, L is

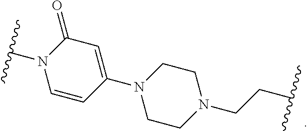 [SMILES: *CCN1CCN(c2ccn(*)c(=O)c2)CC1]

In some embodiments, L is

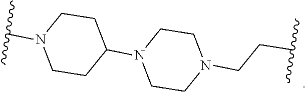 [SMILES: *CCN1CCN(C2CCN(*)CC2)CC1]

In some embodiments, L is

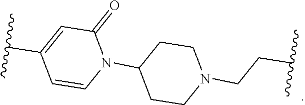 [SMILES: *CCN1CCC(n2ccc(*)cc2=O)CC1]

In some embodiments, L is

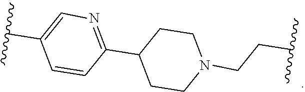 [SMILES: *CCN1CCC(c2ccc(*)cn2)CC1]

In some embodiments, L is

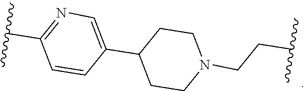 [SMILES: *CCN1CCC(c2ccc(*)nc2)CC1]

In some embodiments, L is

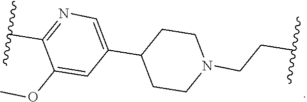 [SMILES: *CCN1CCC(c2cnc(*)c(OC)c2)CC1]

In some embodiments, L is

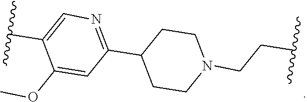 [SMILES: *CCN1CCC(c2cc(OC)c(*)cn2)CC1]

In some embodiments, L is

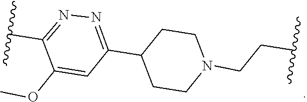 [SMILES: *CCN1CCC(c2cc(OC)c(*)nn2)CC1]

In some embodiments, L is

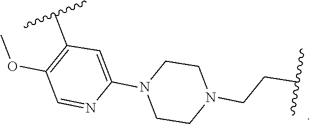 [SMILES: *CCN1CCN(c2cc(*)c(OC)cn2)CC1]

In some embodiments, L is

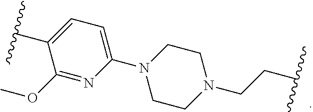 [SMILES: *CCN1CCN(c2ccc(*)c(OC)n2)CC1]

In some embodiments, L is

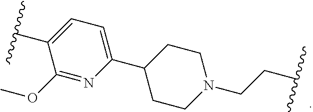 [SMILES: *CCN1CCC(c2ccc(*)c(OC)n2)CC1]

In some embodiments, L is

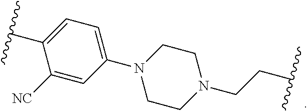 [SMILES: *CCN1CCN(c2ccc(*)c(C#N)c2)CC1]

In some embodiments, L is

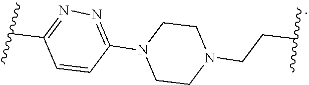 [SMILES: *CCN1CCN(c2ccc(*)nn2)CC1]

In some embodiments, L is

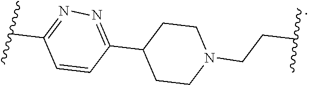 [SMILES: *CCN1CCC(c2ccc(*)nn2)CC1]

In some embodiments, L is

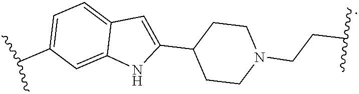 [SMILES: *CCN1CCC(c2cc3ccc(*)cc3[nH]2)CC1]

In some embodiments, L is

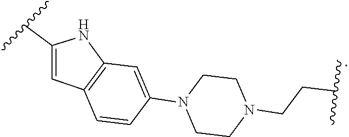 [SMILES: *CCN1CCN(c2ccc3cc(*)[nH]c3c2)CC1]

In some embodiments, L is

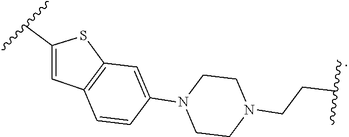 [SMILES: *CCN1CCN(c2ccc3cc(*)sc3c2)CC1]

In some embodiments, L is

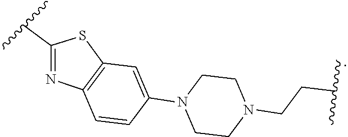 [SMILES: *CCN1CCN(c2ccc3nc(*)sc3c2)CC1]

In some embodiments, L is

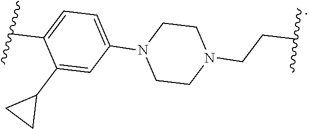 [SMILES: *CCN1CCN(c2ccc(*)c(C3CC3)c2)CC1]

In some embodiments, L is

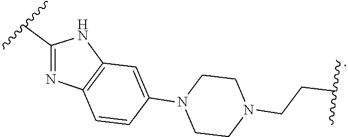 [SMILES: *CCN1CCN(c2ccc3nc(*)[nH]c3c2)CC1]

In some embodiments, L is

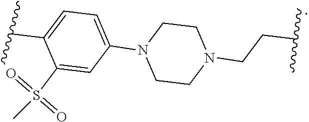 [SMILES: *CCN1CCN(c2ccc(*)c(S(C)(=O)=O)c2)CC1]

In some embodiments, L is

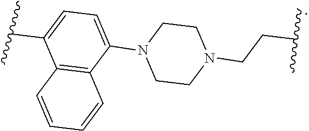 [SMILES: *CCN1CCN(c2ccc(*)c3ccccc23)CC1]

In some embodiments, L is

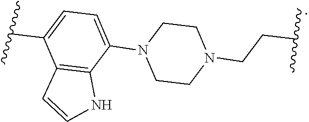 [SMILES: *CCN1CCN(c2ccc(*)c3cc[nH]c23)CC1]

In some embodiments, L is

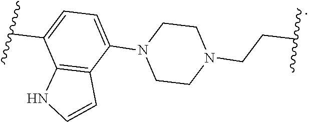 [SMILES: *CCN1CCN(c2ccc(*)c3[nH]ccc23)CC1]

In some embodiments, L is

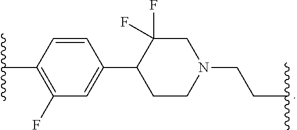 [SMILES: *CCN1CCC(c2ccc(*)c(F)c2)C(F)(F)C1]

In some embodiments, L is

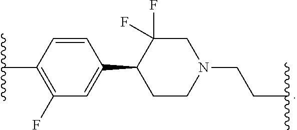 [SMILES: *CCN1CC[C@@H](c2ccc(*)c(F)c2)C(F)(F)C1]

In some embodiments, L is

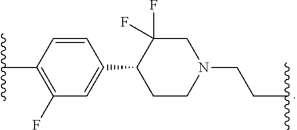 [SMILES: *CCN1CC[C@H](c2ccc(*)c(F)c2)C(F)(F)C1]

In some embodiments, L is

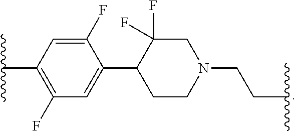 [SMILES: *CCN1CCC(c2cc(F)c(*)cc2F)C(F)(F)C1]

In some embodiments, L is F

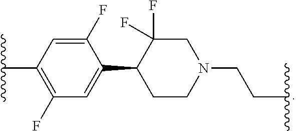 [SMILES: *CCN1CC[C@@H](c2cc(F)c(*)cc2F)C(F)(F)C1]

In some embodiments, L is

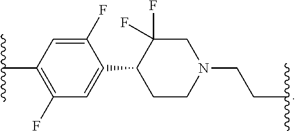 [SMILES: *CCN1CC[C@H](c2cc(F)c(*)cc2F)C(F)(F)C1]

In some embodiments, L is

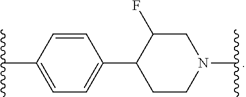 [SMILES: *c1ccc(C2CCN(*)CC2F)cc1]

In some embodiments, L is

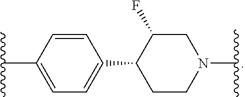 [SMILES: *c1ccc([C@H]2CCN(*)C[C@H]2F)cc1]

In some embodiments, L is

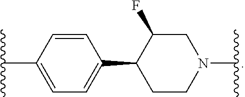 [SMILES: *c1ccc([C@@H]2CCN(*)C[C@@H]2F)cc1]

In some embodiments, L is

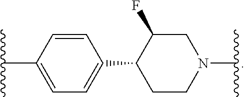 [SMILES: *c1ccc([C@H]2CCN(*)C[C@@H]2F)cc1]

In some embodiments, L is

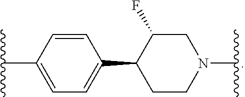 [SMILES: *c1ccc([C@@H]2CCN(*)C[C@H]2F)cc1]

In some embodiments, L is

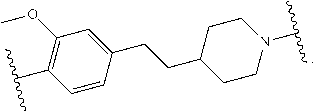 [SMILES: *c1ccc(CCC2CCN(*)CC2)cc1OC]

In some embodiments, L is

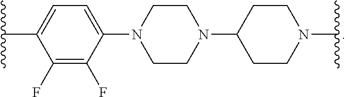 [SMILES: *c1ccc(N2CCN(C3CCN(*)CC3)CC2)c(F)c1F]

In some embodiments, L is

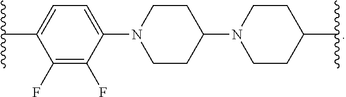 [SMILES: *c1ccc(N2CCC(N3CCC(*)CC3)CC2)c(F)c1F]

In some embodiments, L is selected from those depicted in Table 1, below.

In some embodiments, a provided compound or pharmaceutically acceptable salt thereof, is selected from those wherein SBM is

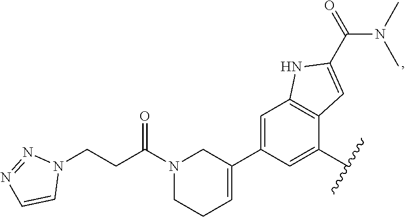 [SMILES: *c1cc(C2=CCCN(C(=O)CCn3ccnn3)C2)cc2[nH]c(C(=O)N(C)C)cc12]

LBM is selected from any of those in Table A below, and L is selected from any of those in Table B below.

In some embodiments, a provided compound or pharmaceutically acceptable salt thereof, is selected from those wherein SBM is

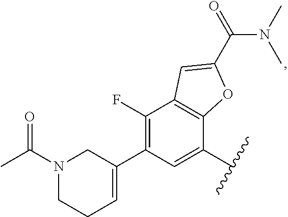 [SMILES: *c1cc(C2=CCCN(C(C)=O)C2)c(F)c2cc(C(=O)N(C)C)oc12]

LBM is selected from any of those in Table A below, and L is selected from any of those in Table B below.

In some embodiments, a provided compound or pharmaceutically acceptable salt thereof, is selected from those wherein SBM is

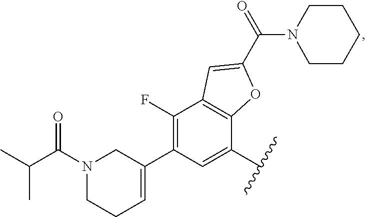 [SMILES: *c1cc(C2=CCCN(C(=O)C(C)C)C2)c(F)c2cc(C(=O)N3CCCCC3)oc12]

LBM is selected from any of those in Table A below, and L is selected from any of those in Table B below.

In some embodiments, a provided compound or pharmaceutically acceptable salt thereof, is selected from those wherein SBM is

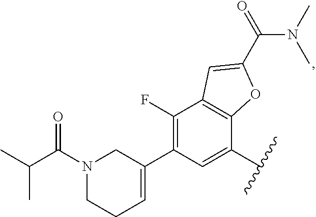 [SMILES: *c1cc(C2=CCCN(C(=O)C(C)C)C2)c(F)c2cc(C(=O)N(C)C)oc12]

LBM is selected from any of those in Table A below, and L is selected from any of those in Table B below.

In some embodiments, a provided compound or pharmaceutically acceptable salt thereof, is selected from those wherein SBM is

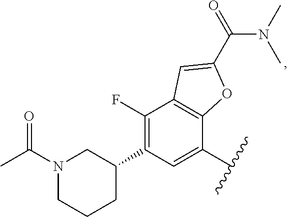 [SMILES: *c1cc([C@@H]2CCCN(C(C)=O)C2)c(F)c2cc(C(=O)N(C)C)oc12]

LBM is selected from any of those in Table A below, and L is selected from any of those in Table B below.

In some embodiments, a provided compound or pharmaceutically acceptable salt thereof, is selected from those wherein SBM is

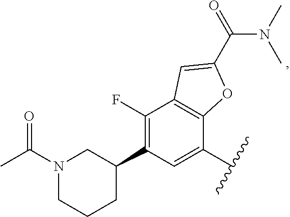 [SMILES: *c1cc([C@H]2CCCN(C(C)=O)C2)c(F)c2cc(C(=O)N(C)C)oc12]

LBM is selected from any of those in Table A below, and L is selected from any of those in Table B below.

In some embodiments, a provided compound or pharmaceutically acceptable salt thereof, is selected from those wherein SBM is

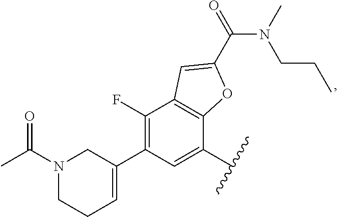 [SMILES: *c1cc(C2=CCCN(C(C)=O)C2)c(F)c2cc(C(=O)N(C)CCC)oc12]

LBM is selected from any of those in Table A below, and L is selected from any of those in Table B below.

In some embodiments, a provided compound or pharmaceutically acceptable salt thereof, is selected from those wherein SBM is

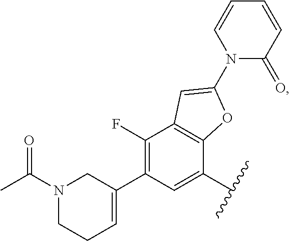 [SMILES: *c1cc(C2=CCCN(C(C)=O)C2)c(F)c2cc(-n3ccccc3=O)oc12]

LBM is selected from any of those in Table A below, and L is selected from any of those in Table B below.

In some embodiments, a provided compound or pharmaceutically acceptable salt thereof, is selected from those wherein SBM is

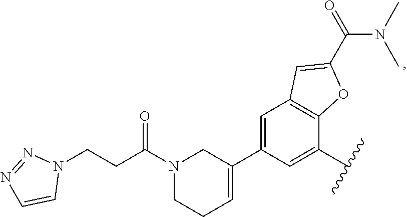 [SMILES: *c1cc(C2=CCCN(C(=O)CCn3ccnn3)C2)cc2cc(C(=O)N(C)C)oc12]

LBM is selected from any of those in Table A below, and L is selected from any of those in Table B below.

In some embodiments, a provided compound or pharmaceutically acceptable salt thereof, is selected from those wherein SBM is

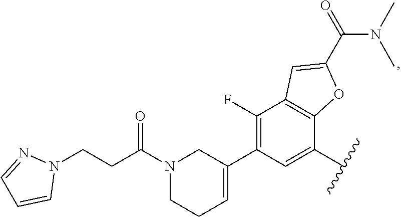 [SMILES: *c1cc(C2=CCCN(C(=O)CCn3cccn3)C2)c(F)c2cc(C(=O)N(C)C)oc12]

LBM is selected from any of those in Table A below, and L is selected from any of those in Table B below.

In some embodiments, a provided compound or pharmaceutically acceptable salt thereof, is selected from those wherein SBM is

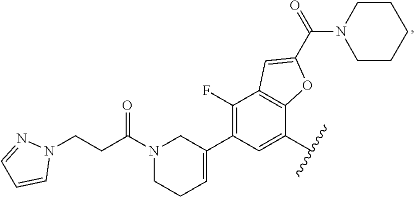 [SMILES: *c1cc(C2=CCCN(C(=O)CCn3cccn3)C2)c(F)c2cc(C(=O)N3CCCCC3)oc12]

LBM is selected from any of those in Table A below, and L is selected from any of those in Table B below.

In some embodiments, a provided compound or pharmaceutical y acceptable salt thereof, is selected from those wherein SBM is

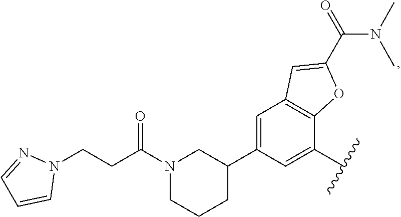 [SMILES: *c1cc(C2CCCN(C(=O)CCn3cccn3)C2)cc2cc(C(=O)N(C)C)oc12]

LBM is selected from any of those in Table A below, and L is selected from any of those in Table B below.

In some embodiments, a provided compound or pharmaceutically acceptable salt thereof, is selected from those wherein SBM is

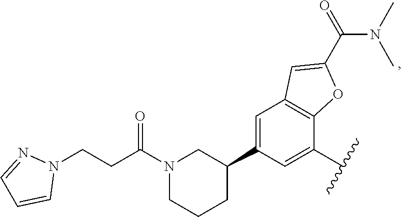 [SMILES: *c1cc([C@H]2CCCN(C(=O)CCn3cccn3)C2)cc2cc(C(=O)N(C)C)oc12]

LBM is selected from any of those in Table A below, and L is selected from any of those in Table B below.

In some embodiments, a provided compound or pharmaceutically acceptable salt thereof, is selected from those wherein SBM is

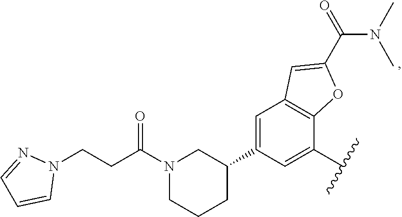 [SMILES: *c1cc([C@@H]2CCCN(C(=O)CCn3cccn3)C2)cc2cc(C(=O)N(C)C)oc12]

LBM is selected from any of those in Table A below, and L is selected from any of those in Table B below.

In some embodiments, a provided compound or pharmaceutically acceptable salt thereof, is selected from those wherein SBM is

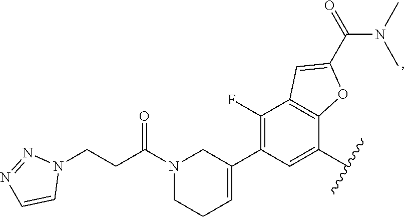 [SMILES: *c1cc(C2=CCCN(C(=O)CCn3ccnn3)C2)c(F)c2cc(C(=O)N(C)C)oc12]

LBM is selected from any of those in Table A below, and L is selected from any of those in Table B below.

In some embodiments, a provided compound or pharmaceutically acceptable salt thereof, is selected from those wherein SBM is

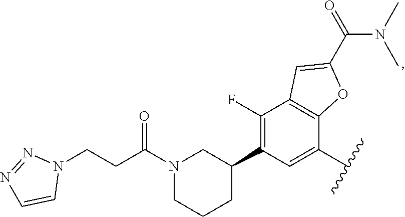 [SMILES: *c1cc([C@H]2CCCN(C(=O)CCn3ccnn3)C2)c(F)c2cc(C(=O)N(C)C)oc12]

LBM is selected from any of those in Table A below, and L is selected from any of those in Table B below.

In some embodiments, a provided compound or pharmaceutically acceptable salt thereof, is selected from those wherein SBM is

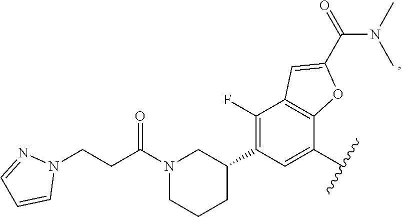 [SMILES: *c1cc([C@@H]2CCCN(C(=O)CCn3cccn3)C2)c(F)c2cc(C(=O)N(C)C)oc12]

LBM is selected from any of those in Table A below, and L is selected from any of those in Table B below.

In some embodiments, a provided compound or pharmaceutically acceptable salt thereof, is selected from those wherein SBM is

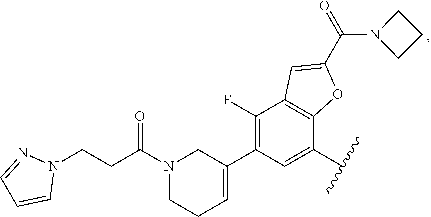 [SMILES: *c1cc(C2=CCCN(C(=O)CCn3cccn3)C2)c(F)c2cc(C(=O)N3CCC3)oc12]

LBM is selected from any of those in Table A below, and L is selected from any of those in Table B below.

In some embodiments, a provided compound or pharmaceutically acceptable salt thereof, is selected from those wherein SBM is

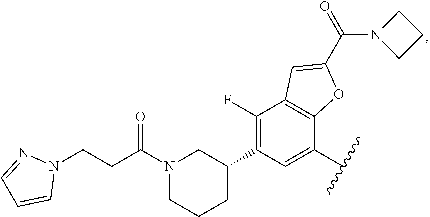 [SMILES: *c1cc([C@@H]2CCCN(C(=O)CCn3cccn3)C2)c(F)c2cc(C(=O)N3CCC3)oc12]

LBM is selected from any of those in Table A below, and L is selected from any of those in Table B below.

In some embodiments, a provided compound or pharmaceutically acceptable salt thereof, is selected from those wherein SBM is

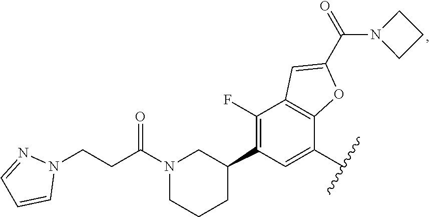 [SMILES: *c1cc([C@H]2CCCN(C(=O)CCn3cccn3)C2)c(F)c2cc(C(=O)N3CCC3)oc12]

LBM is selected from any of those in Table A below, and L is selected from any of those in Table B below.

In some embodiments, a provided compound or pharmaceutically acceptable salt thereof, is selected from those wherein SBM is

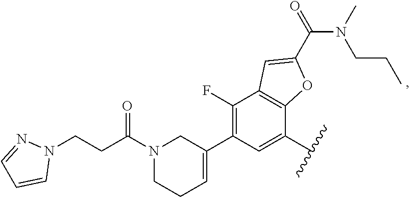 [SMILES: *c1cc(C2=CCCN(C(=O)CCn3cccn3)C2)c(F)c2cc(C(=O)N(C)CCC)oc12]

LBM is selected from any of those in Table A below, and L is selected from any of those in Table B below.

In some embodiments, a provided compound or pharmaceutically acceptable salt thereof, is selected from those wherein SBM is

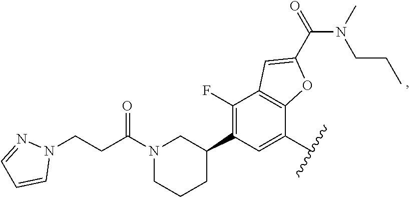 [SMILES: *c1cc([C@H]2CCCN(C(=O)CCn3cccn3)C2)c(F)c2cc(C(=O)N(C)CCC)oc12]

LBM is selected from any of those in Table A below, and L is selected from any of those in Table B below.

In some embodiments, a provided compound or pharmaceutically acceptable salt thereof, is selected from those wherein SBM is

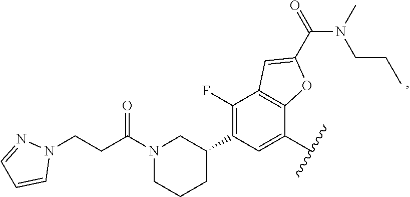 [SMILES: *c1cc([C@@H]2CCCN(C(=O)CCn3cccn3)C2)c(F)c2cc(C(=O)N(C)CCC)oc12]

LBM is selected from any of those in Table A below, and L is selected from any of those in Table B below.

In some embodiments, a provided compound or pharmaceutically acceptable salt thereof, is selected from those wherein SBM is

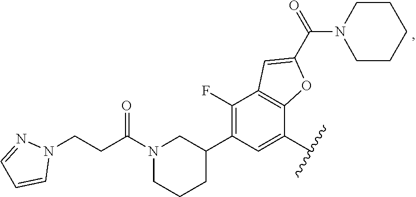 [SMILES: *c1cc(C2CCCN(C(=O)CCn3cccn3)C2)c(F)c2cc(C(=O)N3CCCCC3)oc12]

LBM is selected from any of those in Table A below, and L is selected from any of those in Table B below.

In some embodiments, a provided compound or pharmaceutically acceptable salt thereof, is selected from those wherein SBM is

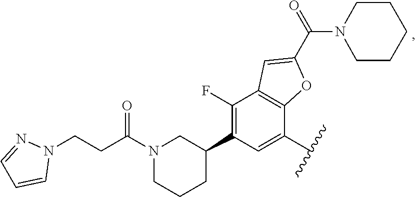 [SMILES: *c1cc([C@H]2CCCN(C(=O)CCn3cccn3)C2)c(F)c2cc(C(=O)N3CCCCC3)oc12]

LBM is selected from any of those in Table A below, and L is selected from any of those in Table B below.

In some embodiments, a provided compound or pharmaceutically acceptable salt thereof, is selected from those wherein SBM is

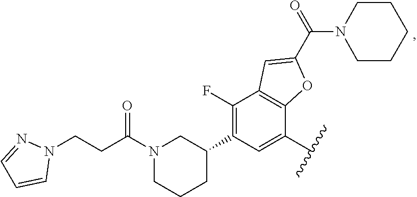 [SMILES: *c1cc([C@@H]2CCCN(C(=O)CCn3cccn3)C2)c(F)c2cc(C(=O)N3CCCCC3)oc12]

LBM is selected from any of those in Table A below, and L is selected from any of those in Table B below.

In some embodiments, a provided compound or pharmaceutically acceptable salt thereof, is selected from those wherein SBM is

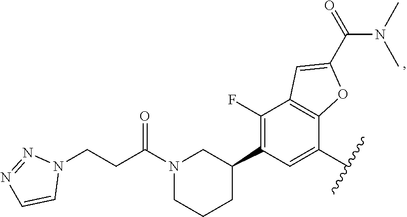 [SMILES: *c1cc([C@H]2CCCN(C(=O)CCn3ccnn3)C2)c(F)c2cc(C(=O)N(C)C)oc12]

LBM is selected from any of those in Table A below, and L is selected from any of those in Table B below.

In some embodiments, a provided compound or pharmaceutically acceptable salt thereof, is selected from those wherein SBM is

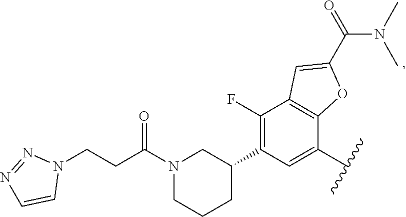 [SMILES: *c1cc([C@@H]2CCCN(C(=O)CCn3ccnn3)C2)c(F)c2cc(C(=O)N(C)C)oc12]

LBM is selected from any of those in Table A below, and L is selected from any of those in Table B below.

In some embodiments, a provided compound or pharmaceutically acceptable salt thereof, is selected from those wherein SBM is

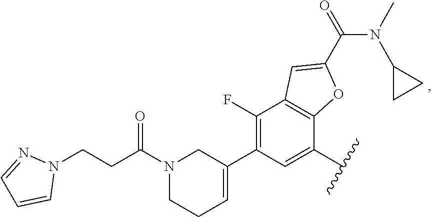 [SMILES: *c1cc(C2=CCCN(C(=O)CCn3cccn3)C2)c(F)c2cc(C(=O)N(C)C3CC3)oc12]

LBM is selected from any of those in Table A below, and L is selected from any of those in Table B below.

In some embodiments, a provided compound or pharmaceutically acceptable salt thereof, is selected from those wherein SBM is

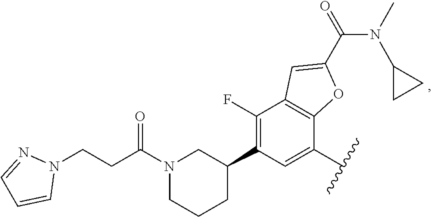 [SMILES: *c1cc([C@H]2CCCN(C(=O)CCn3cccn3)C2)c(F)c2cc(C(=O)N(C)C3CC3)oc12]

LBM is selected from any of those in Table A below, and L is selected from any of those in Table B below.

In some embodiments, a provided compound or pharmaceutically acceptable salt thereof, is selected from those wherein SBM is

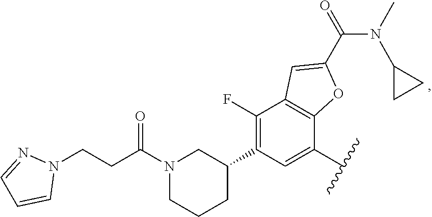 [SMILES: *c1cc([C@@H]2CCCN(C(=O)CCn3cccn3)C2)c(F)c2cc(C(=O)N(C)C3CC3)oc12]

LBM is selected from any of those in Table A below, and L is selected from any of those in Table B below.

In some embodiments, a provided compound or pharmaceutically acceptable salt thereof, is selected from those wherein SBM is

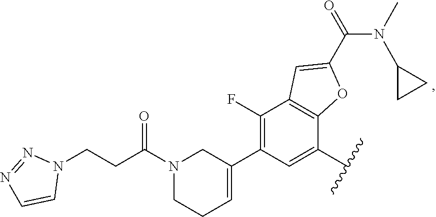 [SMILES: *c1cc(C2=CCCN(C(=O)CCn3ccnn3)C2)c(F)c2cc(C(=O)N(C)C3CC3)oc12]

LBM is selected from any of those in Table A below, and L is selected from any of those in Table B below.

In some embodiments, a provided compound or pharmaceutically acceptable salt thereof, is selected from those wherein SBM is

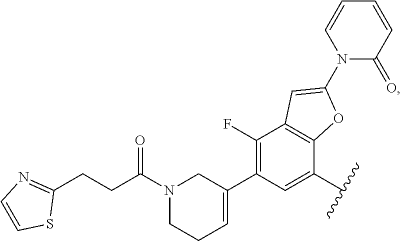 [SMILES: *c1cc(C2=CCCN(C(=O)CCc3nccs3)C2)c(F)c2cc(-n3ccccc3=O)oc12]

LBM is selected from any of those in Table A below, and L is selected from any of those in Table B below.

In some embodiments, a provided compound or pharmaceutically acceptable salt thereof, is selected from those wherein SBM is

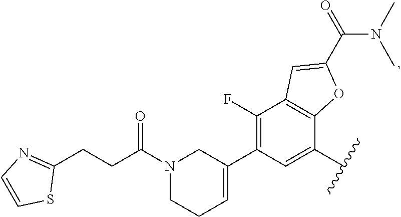 [SMILES: *c1cc(C2=CCCN(C(=O)CCc3nccs3)C2)c(F)c2cc(C(=O)N(C)C)oc12]

LBM is selected from any of those in Table A below, and L is selected from any of those in Table B below.

In some embodiments, a provided compound or pharmaceutically acceptable salt thereof, is selected from those wherein SBM is

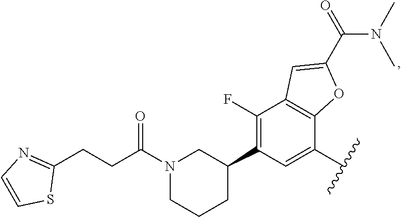 [SMILES: *c1cc([C@H]2CCCN(C(=O)CCc3nccs3)C2)c(F)c2cc(C(=O)N(C)C)oc12]

LBM is selected from any of those in Table A below, and L is selected from any of those in Table B below.

In some embodiments, a provided compound or pharmaceutically acceptable salt thereof, is selected from those wherein SBM is

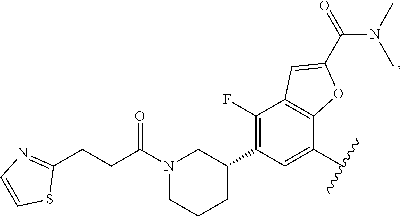 [SMILES: *c1cc([C@@H]2CCCN(C(=O)CCc3nccs3)C2)c(F)c2cc(C(=O)N(C)C)oc12]

LBM is selected from any of those in Table A below, and L is selected from any of those in Table B below.

In some embodiments, a provided compound or pharmaceutically acceptable salt thereof, is selected from those wherein SBM is

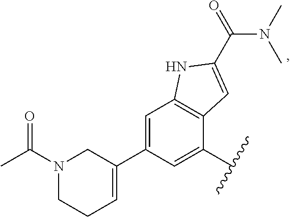 [SMILES: *c1cc(C2=CCCN(C(C)=O)C2)cc2[nH]c(C(=O)N(C)C)cc12]

LBM is selected from any of those in Table A below, and L is selected from any of those in Table B below.

In some embodiments, a provided compound or pharmaceutically acceptable salt thereof, is selected from those wherein SBM is

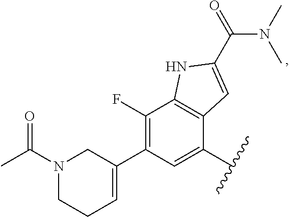 [SMILES: *c1cc(C2=CCCN(C(C)=O)C2)c(F)c2[nH]c(C(=O)N(C)C)cc12]

LBM is selected from any of those in Table A below, and L is selected from any of those in Table B below.

In some embodiments, a provided compound or pharmaceutically acceptable salt thereof, is selected from those wherein SBM is

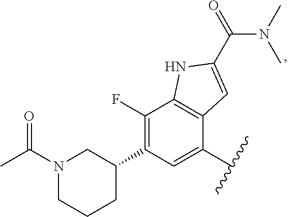 [SMILES: *c1cc([C@@H]2CCCN(C(C)=O)C2)c(F)c2[nH]c(C(=O)N(C)C)cc12]

LBM is selected from any of those in Table A below, and L is selected from any of those in Table B below.

In some embodiments, a provided compound or pharmaceutically acceptable salt thereof, is selected from those wherein SBM is

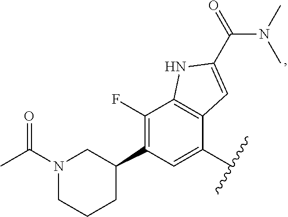 [SMILES: *c1cc([C@H]2CCCN(C(C)=O)C2)c(F)c2[nH]c(C(=O)N(C)C)cc12]

LBM is selected from any of those in Table A below, and L is selected from any of those in Table B below.

In some embodiments, a provided compound or pharmaceutically acceptable salt thereof, is selected from those wherein SBM is

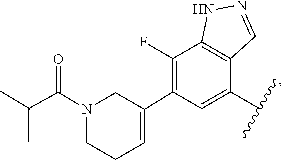 [SMILES: *c1cc(C2=CCCN(C(=O)C(C)C)C2)c(F)c2[nH]ncc12]

LBM is selected from any of those in Table A below, and L is selected from any of those in Table B below.

In some embodiments, a provided compound or pharmaceutically acceptable salt thereof, is selected from those wherein SBM is

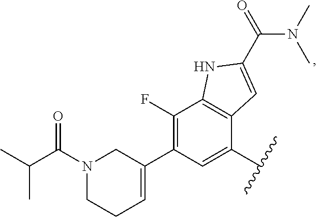 [SMILES: *c1cc(C2=CCCN(C(=O)C(C)C)C2)c(F)c2[nH]c(C(=O)N(C)C)cc12]

LBM is selected from any of those in Table A below, and L is selected from any of those in Table B below.

In some embodiments, a provided compound or pharmaceutically acceptable salt thereof, is selected from those wherein SBM is

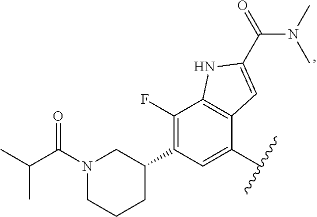 [SMILES: *c1cc([C@@H]2CCCN(C(=O)C(C)C)C2)c(F)c2[nH]c(C(=O)N(C)C)cc12]

LBM is selected from any of those in Table A below, and L is selected from any of those in Table B below.

In some embodiments, a provided compound or pharmaceutically acceptable salt thereof, is selected from those wherein SBM is

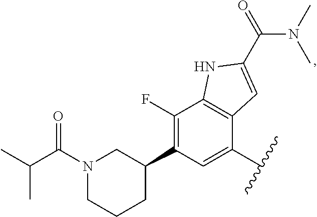 [SMILES: *c1cc([C@H]2CCCN(C(=O)C(C)C)C2)c(F)c2[nH]c(C(=O)N(C)C)cc12]

LBM is selected from any of those in Table A below, and L is selected from any of those in Table B below.

In some embodiments, a provided compound or pharmaceutically acceptable salt thereof, is selected from those wherein SBM is

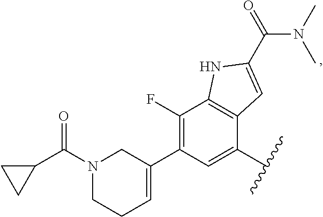 [SMILES: *c1cc(C2=CCCN(C(=O)C3CC3)C2)c(F)c2[nH]c(C(=O)N(C)C)cc12]

LBM is selected from any of those in Table A below, and L is selected from any of those in Table B below.

In some embodiments, a provided compound or pharmaceutically acceptable salt thereof, is selected from those wherein SBM is

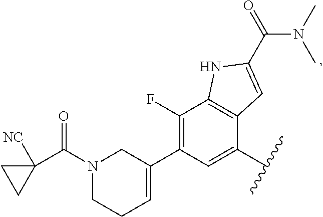 [SMILES: *c1cc(C2=CCCN(C(=O)C3(C#N)CC3)C2)c(F)c2[nH]c(C(=O)N(C)C)cc12]

LBM is selected from any of those in Table A below, and L is selected from any of those in Table B below.

In some embodiments, a provided compound or pharmaceutically acceptable salt thereof, is selected from those wherein SBM is

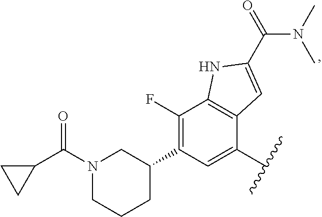 [SMILES: *c1cc([C@@H]2CCCN(C(=O)C3CC3)C2)c(F)c2[nH]c(C(=O)N(C)C)cc12]

LBM is selected from any of those in Table A below, and L is selected from any of those in Table B below.

In some embodiments, a provided compound or pharmaceutically acceptable salt thereof, is selected from those wherein SBM is

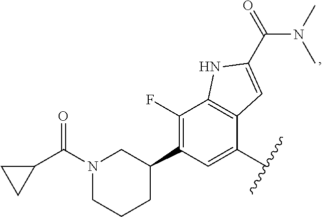 [SMILES: *c1cc([C@H]2CCCN(C(=O)C3CC3)C2)c(F)c2[nH]c(C(=O)N(C)C)cc12]

LBM is selected from any of those in Table A below, and L is selected from any of those in Table B below.

In some embodiments, a provided compound or pharmaceutically acceptable salt thereof, is selected from those wherein SBM is

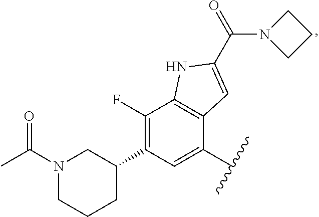 [SMILES: *c1cc([C@@H]2CCCN(C(C)=O)C2)c(F)c2[nH]c(C(=O)N3CCC3)cc12]

LBM is selected from any of those in Table A below, and L is selected from any of those in Table B below.

In some embodiments, a provided compound or pharmaceutically acceptable salt thereof, is selected from those wherein SBM is

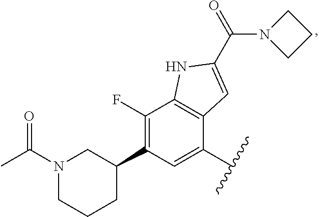 [SMILES: *c1cc([C@H]2CCCN(C(C)=O)C2)c(F)c2[nH]c(C(=O)N3CCC3)cc12]

LBM is selected from any of those in Table A below, and L is selected from any of those in Table B below.

In some embodiments, a provided compound or pharmaceutically acceptable salt thereof, is selected from those wherein SBM is

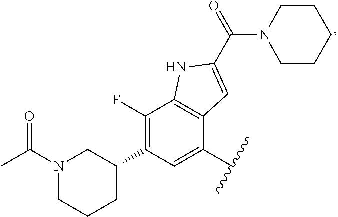 [SMILES: *c1cc([C@@H]2CCCN(C(C)=O)C2)c(F)c2[nH]c(C(=O)N3CCCCC3)cc12]

LBM is selected from any of those in Table A below, and L is selected from any of those in Table B below.

In some embodiments, a provided compound or pharmaceutically acceptable salt thereof, is selected from those wherein SBM is

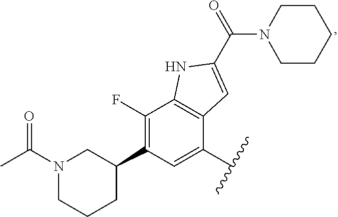 [SMILES: *c1cc([C@H]2CCCN(C(C)=O)C2)c(F)c2[nH]c(C(=O)N3CCCCC3)cc12]

LBM is selected from any of those in Table A below, and L is selected from any of those in Table B below.

In some embodiments, a provided compound or pharmaceutically acceptable salt thereof, is selected from those wherein SBM is

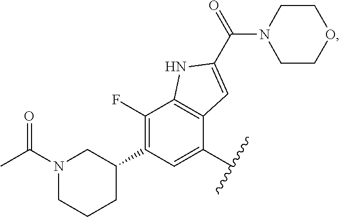 [SMILES: *c1cc([C@@H]2CCCN(C(C)=O)C2)c(F)c2[nH]c(C(=O)N3CCOCC3)cc12]

LBM is selected from any of those in Table A below, and L is selected from any of those in Table B below.

In some embodiments, a provided compound or pharmaceutically acceptable salt thereof, is selected from those wherein SBM is

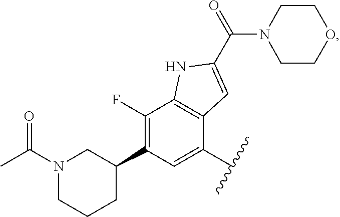 [SMILES: *c1cc([C@H]2CCCN(C(C)=O)C2)c(F)c2[nH]c(C(=O)N3CCOCC3)cc12]

LBM is selected from any of those in Table A below, and L is selected from any of those in Table B below.

In some embodiments, a provided compound or pharmaceutically acceptable salt thereof, is selected from those wherein SBM is

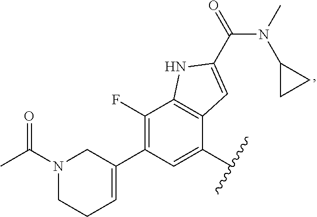 [SMILES: *c1cc(C2=CCCN(C(C)=O)C2)c(F)c2[nH]c(C(=O)N(C)C3CC3)cc12]

LBM is selected from any of those in Table A below, and L is selected from any of those in Table B below.

In some embodiments, a provided compound or pharmaceutically acceptable salt thereof, is selected from those wherein SBM is

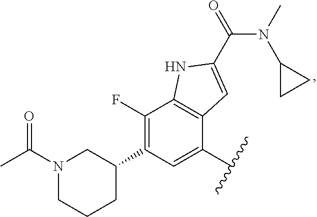 [SMILES: *c1cc([C@@H]2CCCN(C(C)=O)C2)c(F)c2[nH]c(C(=O)N(C)C3CC3)cc12]

LBM is selected from any of those in Table A below, and L is selected from any of those in Table B below.

In some embodiments, a provided compound or pharmaceutically acceptable salt thereof, is selected from those wherein SBM is

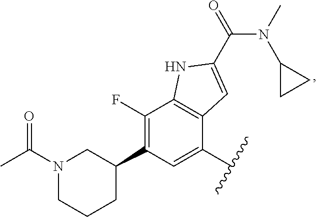 [SMILES: *c1cc([C@H]2CCCN(C(C)=O)C2)c(F)c2[nH]c(C(=O)N(C)C3CC3)cc12]

LBM is selected from any of those in Table A below, and L is selected from any of those in Table B below.

In some embodiments, a provided compound or pharmaceutically acceptable salt thereof, is selected from those wherein SBM is

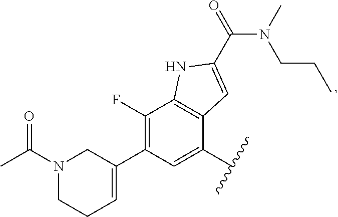 [SMILES: *c1cc(C2=CCCN(C(C)=O)C2)c(F)c2[nH]c(C(=O)N(C)CCC)cc12]

LBM is selected from any of those in Table A below, and L is selected from any of those in Table B below.

In some embodiments, a provided compound or pharmaceutically acceptable salt thereof, is selected from those wherein SBM is

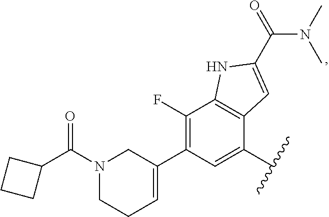 [SMILES: *c1cc(C2=CCCN(C(=O)C3CCC3)C2)c(F)c2[nH]c(C(=O)N(C)C)cc12]

LBM is selected from any of those in Table A below, and L is selected from any of those in Table B below.

In some embodiments, a provided compound or pharmaceutically acceptable salt thereof, is selected from those wherein SBM is

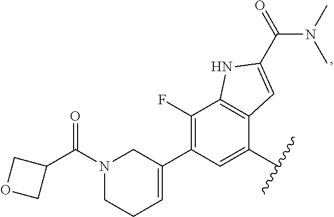 [SMILES: *c1cc(C2=CCCN(C(=O)C3COC3)C2)c(F)c2[nH]c(C(=O)N(C)C)cc12]

LBM is selected from any of those in Table A below, and L is selected from any of those in Table B below.

In some embodiments, a provided compound or pharmaceutically acceptable salt thereof, is selected from those wherein SBM is

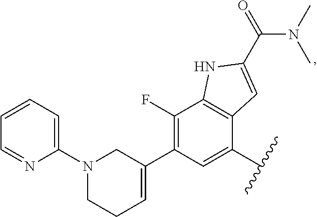 [SMILES: *c1cc(C2=CCCN(c3ccccn3)C2)c(F)c2[nH]c(C(=O)N(C)C)cc12]

LBM is selected from any of those in Table A below, and L is selected from any of those in Table B below.

In some embodiments, a provided compound or pharmaceutically acceptable salt thereof, is selected from those wherein SBM is

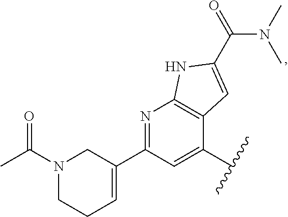 [SMILES: *c1cc(C2=CCCN(C(C)=O)C2)nc2[nH]c(C(=O)N(C)C)cc12]

LBM is selected from any of those in Table A below, and L is selected from any of those in Table B below.

In some embodiments, a provided compound or pharmaceutically acceptable salt thereof, is selected from those wherein SBM is

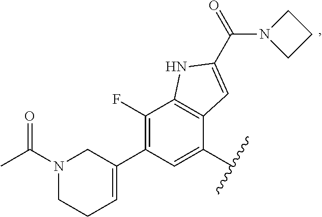 [SMILES: *c1cc(C2=CCCN(C(C)=O)C2)c(F)c2[nH]c(C(=O)N3CCC3)cc12]

LBM is selected from any of those in Table A below, and L is selected from any of those in Table B below.

In some embodiments, a provided compound or pharmaceutically acceptable salt thereof, is selected from those wherein SBM is

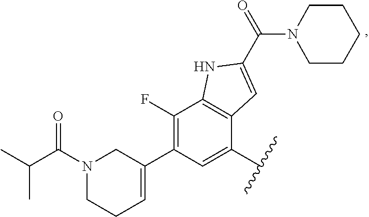 [SMILES: *c1cc(C2=CCCN(C(=O)C(C)C)C2)c(F)c2[nH]c(C(=O)N3CCCCC3)cc12]

LBM is selected from any of those in Table A below, and L is selected from any of those in Table B below.

In some embodiments, a provided compound or pharmaceutically acceptable salt thereof, is selected from those wherein SBM is

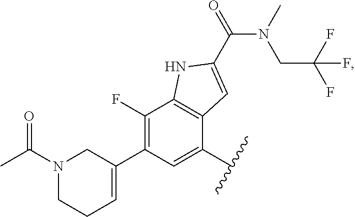 [SMILES: *c1cc(C2=CCCN(C(C)=O)C2)c(F)c2[nH]c(C(=O)N(C)CC(F)(F)F)cc12]

LBM is selected from any of those in Table A below, and L is selected from any of those in Table B below.

In some embodiments, a provided compound or pharmaceutically acceptable salt thereof, is selected from those wherein SBM is

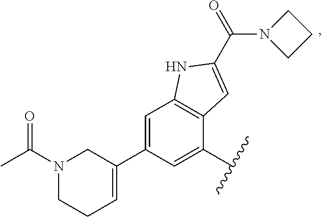 [SMILES: *c1cc(C2=CCCN(C(C)=O)C2)cc2[nH]c(C(=O)N3CCC3)cc12]

LBM is selected from any of those in Table A below, and L is selected from any of those in Table B below.

In some embodiments, a provided compound or pharmaceutically acceptable salt thereof, is selected from those wherein SBM is

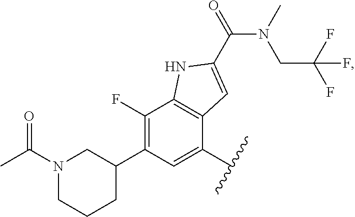 [SMILES: *c1cc(C2CCCN(C(C)=O)C2)c(F)c2[nH]c(C(=O)N(C)CC(F)(F)F)cc12]

LBM is selected from any of those in Table A below, and L is selected from any of those in Table B below.

In some embodiments, a provided compound or pharmaceutically acceptable salt thereof, is selected from those wherein SBM is

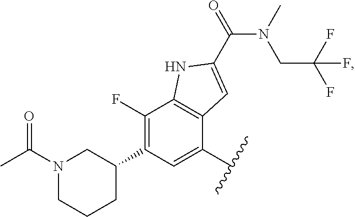 [SMILES: *c1cc([C@@H]2CCCN(C(C)=O)C2)c(F)c2[nH]c(C(=O)N(C)CC(F)(F)F)cc12]

LBM is selected from any of those in Table A below, and L is selected from any of those in Table B below.

In some embodiments, a provided compound or pharmaceutically acceptable salt thereof, is selected from those wherein SBM is

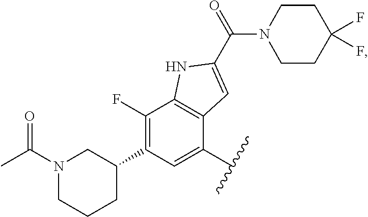 [SMILES: *c1cc([C@@H]2CCCN(C(C)=O)C2)c(F)c2[nH]c(C(=O)N3CCC(F)(F)CC3)cc12]

LBM is selected from any of those in Table A below, and L is selected from any of those in Table B below.

In some embodiments, a provided compound or pharmaceutically acceptable salt thereof, is selected from those wherein SBM is

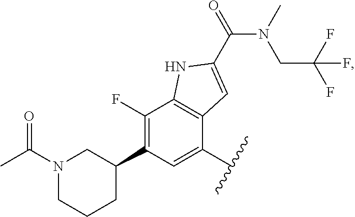 [SMILES: *c1cc([C@H]2CCCN(C(C)=O)C2)c(F)c2[nH]c(C(=O)N(C)CC(F)(F)F)cc12]

LBM is selected from any of those in Table A below, and L is selected from any of those in Table B below.

In some embodiments, a provided compound or pharmaceutically acceptable salt thereof, is selected from those wherein SBM is

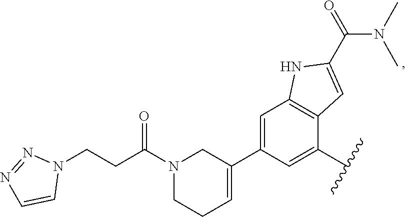 [SMILES: *c1cc(C2=CCCN(C(=O)CCn3ccnn3)C2)cc2[nH]c(C(=O)N(C)C)cc12]

LBM is selected from any of those in Table A below, and L is selected from any of those in Table B below.

In some embodiments, a provided compound or pharmaceutically acceptable salt thereof, is selected from those wherein SBM is

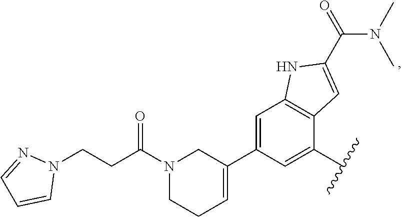 [SMILES: *c1cc(C2=CCCN(C(=O)CCn3cccn3)C2)cc2[nH]c(C(=O)N(C)C)cc12]

LBM is selected from any of those in Table A below, and L is selected from any of those in Table B below.

In some embodiments, a provided compound or pharmaceutically acceptable salt thereof, is selected from those wherein SBM is

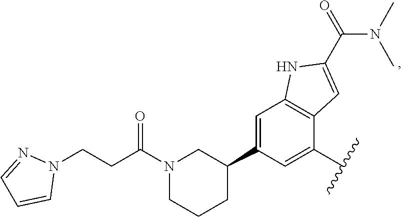 [SMILES: *c1cc([C@H]2CCCN(C(=O)CCn3cccn3)C2)cc2[nH]c(C(=O)N(C)C)cc12]

LBM is selected from any of those in Table A below, and L is selected from any of those in Table B below.

In some embodiments, a provided compound or pharmaceutically acceptable salt thereof, is selected from those wherein SBM is

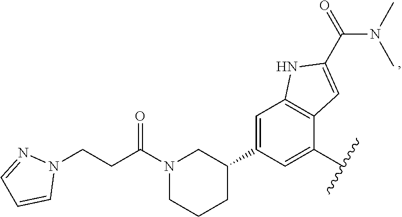 [SMILES: *c1cc([C@@H]2CCCN(C(=O)CCn3cccn3)C2)cc2[nH]c(C(=O)N(C)C)cc12]

LBM is selected from any of those in Table A below, and L is selected from any of those in Table B below.

In some embodiments, a provided compound or pharmaceutically acceptable salt thereof, is selected from those wherein SBM is

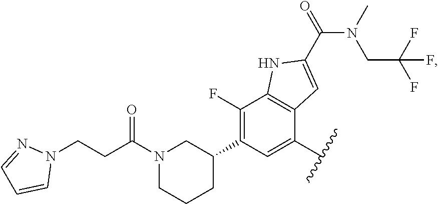 [SMILES: *c1cc([C@@H]2CCCN(C(=O)CCn3cccn3)C2)c(F)c2[nH]c(C(=O)N(C)CC(F)(F)F)cc12]

LBM is selected from any of those in Table A below, and L is selected from any of those in Table B below.

In some embodiments, a provided compound or pharmaceutically acceptable salt thereof, is selected from those wherein SBM is

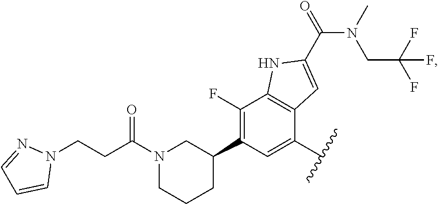 [SMILES: *c1cc([C@H]2CCCN(C(=O)CCn3cccn3)C2)c(F)c2[nH]c(C(=O)N(C)CC(F)(F)F)cc12]

LBM is selected from any of those in Table A below, and L is selected from any of those in Table B below.

In some embodiments, a provided compound or pharmaceutically acceptable salt thereof, is selected from those wherein SBM is

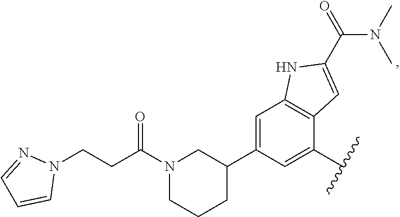 [SMILES: *c1cc(C2CCCN(C(=O)CCn3cccn3)C2)cc2[nH]c(C(=O)N(C)C)cc12]

LBM is selected from any of those in Table A below, and L is selected from any of those in Table B below.

In some embodiments, a provided compound or pharmaceutically acceptable salt thereof, is selected from those wherein SBM is

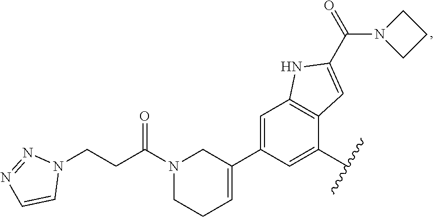 [SMILES: *c1cc(C2=CCCN(C(=O)CCn3ccnn3)C2)cc2[nH]c(C(=O)N3CCC3)cc12]

LBM is selected from any of those in Table A below, and L is selected from any of those in Table B below.

In some embodiments, a provided compound or pharmaceutically acceptable salt thereof, is selected from those wherein SBM is

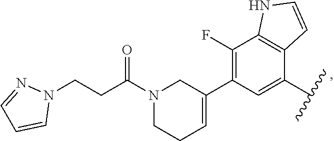 [SMILES: *c1cc(C2=CCCN(C(=O)CCn3cccn3)C2)c(F)c2[nH]ccc12]

LBM is selected from any of those in Table A below, and L is selected from any of those in Table B below.

In some embodiments, a provided compound or pharmaceutically acceptable salt thereof, is selected from those wherein SBM is

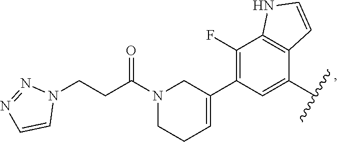 [SMILES: *c1cc(C2=CCCN(C(=O)CCn3ccnn3)C2)c(F)c2[nH]ccc12]

LBM is selected from any of those in Table A below, and L is selected from any of those in Table B below.

In some embodiments, a provided compound or pharmaceutically acceptable salt thereof, is selected from those wherein SBM is

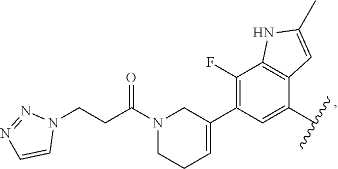 [SMILES: *c1cc(C2=CCCN(C(=O)CCn3ccnn3)C2)c(F)c2[nH]c(C)cc12]

LBM is selected from any of those in Table A below, and L is selected from any of those in Table B below.

In some embodiments, a provided compound or pharmaceutically acceptable salt thereof, is selected from those wherein SBM is

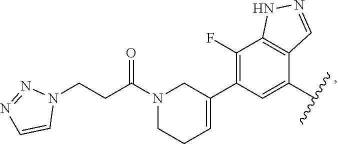 [SMILES: *c1cc(C2=CCCN(C(=O)CCn3ccnn3)C2)c(F)c2[nH]ncc12]

LBM is selected from any of those in Table A below, and L is selected from any of those in Table B below.

In some embodiments, a provided compound or pharmaceutically acceptable salt thereof, is selected from those wherein SBM is

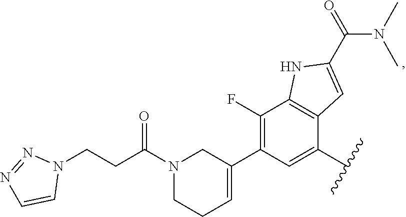 [SMILES: *c1cc(C2=CCCN(C(=O)CCn3ccnn3)C2)c(F)c2[nH]c(C(=O)N(C)C)cc12]

LBM is selected from any of those in Table A below, and L is selected from any of those in Table B below.

In some embodiments, a provided compound or pharmaceutically acceptable salt thereof, is selected from those wherein SBM is

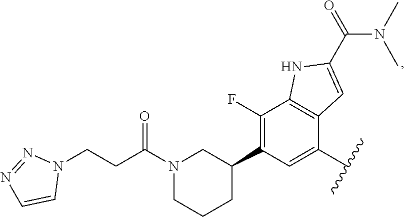 [SMILES: *c1cc([C@H]2CCCN(C(=O)CCn3ccnn3)C2)c(F)c2[nH]c(C(=O)N(C)C)cc12]

LBM is selected from any of those in Table A below, and L is selected from any of those in Table B below.

In some embodiments, a provided compound or pharmaceutically acceptable salt thereof, is selected from those wherein SBM is

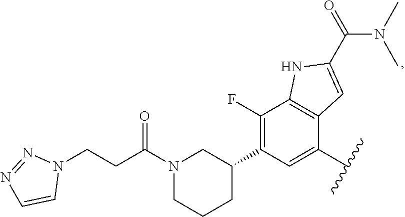 [SMILES: *c1cc([C@@H]2CCCN(C(=O)CCn3ccnn3)C2)c(F)c2[nH]c(C(=O)N(C)C)cc12]

LBM is selected from any of those in Table A below, and L is selected from any of those in Table B below.

In some embodiments, a provided compound or pharmaceutically acceptable salt thereof, is selected from those wherein SBM is

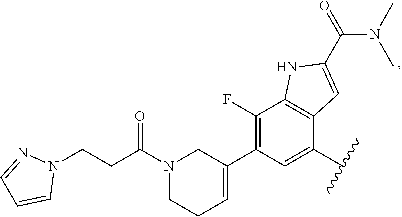 [SMILES: *c1cc(C2=CCCN(C(=O)CCn3cccn3)C2)c(F)c2[nH]c(C(=O)N(C)C)cc12]

LBM is selected from any of those in Table A below, and L is selected from any of those in Table B below.

In some embodiments, a provided compound or pharmaceutically acceptable salt thereof, is selected from those wherein SBM is

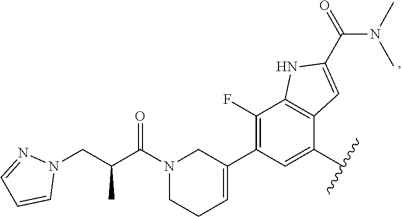 [SMILES: *c1cc(C2=CCCN(C(=O)[C@@H](C)Cn3cccn3)C2)c(F)c2[nH]c(C(=O)N(C)C)cc12]

LBM is selected from any of those in Table A below, and L is selected from any of those in Table B below.

In some embodiments, a provided compound or pharmaceutically acceptable salt thereof, is selected from those wherein SBM is

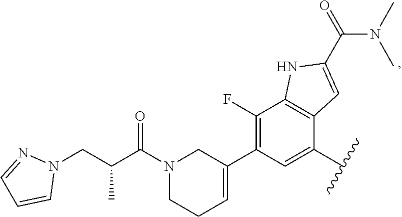 [SMILES: *c1cc(C2=CCCN(C(=O)[C@H](C)Cn3cccn3)C2)c(F)c2[nH]c(C(=O)N(C)C)cc12]

LBM is selected from any of those in Table A below, and L is selected from any of those in Table B below.

In some embodiments, a provided compound or pharmaceutically acceptable salt thereof, is selected from those wherein SBM is

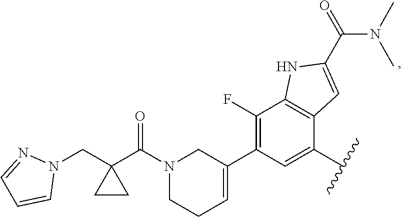 [SMILES: *c1cc(C2=CCCN(C(=O)C3(Cn4cccn4)CC3)C2)c(F)c2[nH]c(C(=O)N(C)C)cc12]

LBM is selected from any of those in Table A below, and L is selected from any of those in Table B below.

In some embodiments, a provided compound or pharmaceutically acceptable salt thereof, is selected from those wherein SBM is

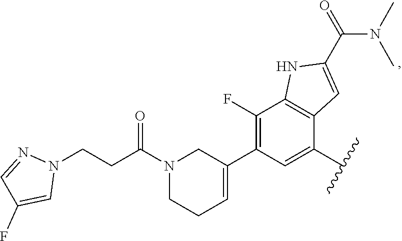 [SMILES: *c1cc(C2=CCCN(C(=O)CCn3cc(F)cn3)C2)c(F)c2[nH]c(C(=O)N(C)C)cc12]

LBM is selected from any of those in Table A below, and L is selected from any of those in Table B below.

In some embodiments, a provided compound or pharmaceutically acceptable salt thereof, is selected from those wherein SBM is

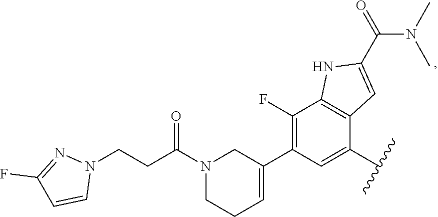 [SMILES: *c1cc(C2=CCCN(C(=O)CCn3ccc(F)n3)C2)c(F)c2[nH]c(C(=O)N(C)C)cc12]

LBM is selected from any of those in Table A below, and L is selected from any of those in Table B below.

In some embodiments, a provided compound or pharmaceutically acceptable salt thereof, is selected from those wherein SBM is

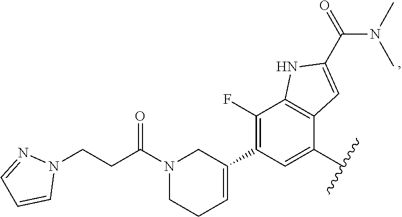

LBM is selected from any of those in Table A below, and L is selected from any of those in Table B below.

In some embodiments, a provided compound or pharmaceutically acceptable salt thereof, is selected from those wherein SBM is

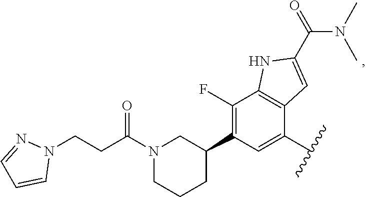 [SMILES: *c1cc([C@H]2CCCN(C(=O)CCn3cccn3)C2)c(F)c2[nH]c(C(=O)N(C)C)cc12]

LBM is selected from any of those in Table A below, and L is selected from any of those in Table B below.

In some embodiments, a provided compound or pharmaceutically acceptable salt thereof, is selected from those wherein SBM is

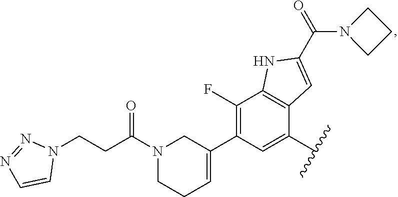 [SMILES: *c1cc(C2=CCCN(C(=O)CCn3ccnn3)C2)c(F)c2[nH]c(C(=O)N3CCC3)cc12]

LBM is selected from any of those in Table A below, and L is selected from any of those in Table B below.

In some embodiments, a provided compound or pharmaceutically acceptable salt thereof, is selected from those wherein SBM is

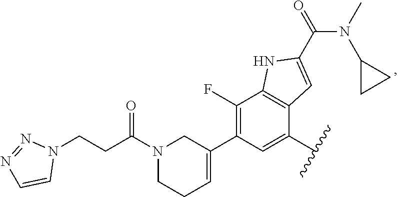 [SMILES: *c1cc(C2=CCCN(C(=O)CCn3ccnn3)C2)c(F)c2[nH]c(C(=O)N(C)C3CC3)cc12]

LBM is selected from any of those in Table A below, and L is selected from any of those in Table B below.

In some embodiments, a provided compound or pharmaceutically acceptable salt thereof, is selected from those wherein SBM is

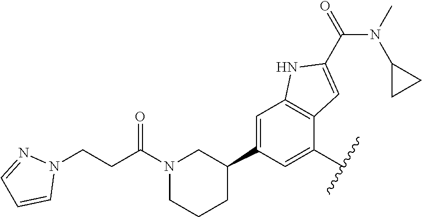 [SMILES: *c1cc([C@H]2CCCN(C(=O)CCn3cccn3)C2)cc2[nH]c(C(=O)N(C)C3CC3)cc12]

LBM is selected from any of those in Table A below, and L is selected from any of those in Table B below.

In some embodiments, a provided compound or pharmaceutically acceptable salt thereof, is selected from those wherein SBM is

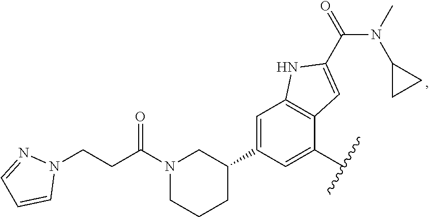 [SMILES: *c1cc([C@@H]2CCCN(C(=O)CCn3cccn3)C2)cc2[nH]c(C(=O)N(C)C3CC3)cc12]

LBM is selected from any of those in Table A below, and L is selected from any of those in Table B below.

In some embodiments, a provided compound or pharmaceutically acceptable salt thereof, is selected from those wherein SBM is

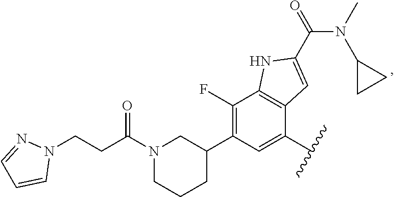 [SMILES: *c1cc(C2CCCN(C(=O)CCn3cccn3)C2)c(F)c2[nH]c(C(=O)N(C)C3CC3)cc12]

LBM is selected from any of those in Table A below, and L is selected from any of those in Table B below.

In some embodiments, a provided compound or pharmaceutically acceptable salt thereof, is selected from those wherein SBM is

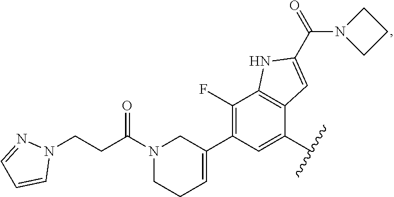 [SMILES: *c1cc(C2=CCCN(C(=O)CCn3cccn3)C2)c(F)c2[nH]c(C(=O)N3CCC3)cc12]

LBM is selected from any of those in Table A below, and L is selected from any of those in Table B below.

In some embodiments, a provided compound or pharmaceutically acceptable salt thereof, is selected from those wherein SBM is

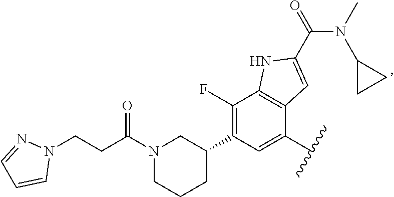 [SMILES: *c1cc([C@@H]2CCCN(C(=O)CCn3cccn3)C2)c(F)c2[nH]c(C(=O)N(C)C3CC3)cc12]

LBM is selected from any of those in Table A below, and L is selected from any of those in Table B below.

In some embodiments, a provided compound or pharmaceutically acceptable salt thereof, is selected from those wherein SBM is

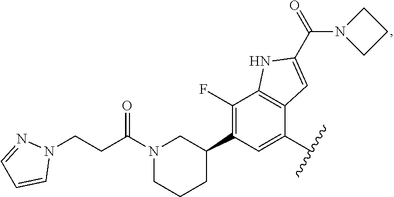 [SMILES: *c1cc([C@H]2CCCN(C(=O)CCn3cccn3)C2)c(F)c2[nH]c(C(=O)N3CCC3)cc12]

LBM is selected from any of those in Table A below, and L is selected from any of those in Table B below.

In some embodiments, a provided compound or pharmaceutically acceptable salt thereof, is selected from those wherein SBM is

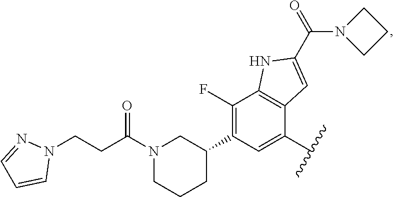 [SMILES: *c1cc([C@@H]2CCCN(C(=O)CCn3cccn3)C2)c(F)c2[nH]c(C(=O)N3CCC3)cc12]

LBM is selected from any of those in Table A below, and L is selected from any of those in Table B below.

In some embodiments, a provided compound or pharmaceutically acceptable salt thereof, is selected from those wherein SBM is

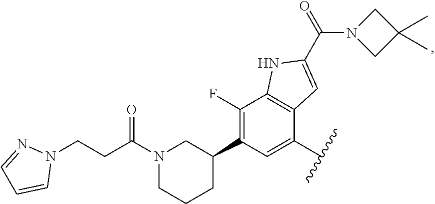 [SMILES: *c1cc([C@H]2CCCN(C(=O)CCn3cccn3)C2)c(F)c2[nH]c(C(=O)N3CC(C)(C)C3)cc12]

LBM is selected from any of those in Table A below, and L is selected from any of those in Table B below.

In some embodiments, a provided compound or pharmaceutically acceptable salt thereof, is selected from those wherein SBM is

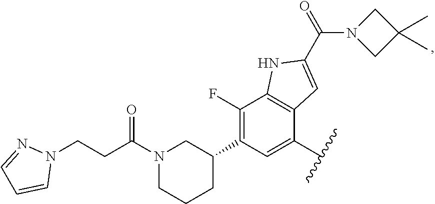 [SMILES: *c1cc([C@@H]2CCCN(C(=O)CCn3cccn3)C2)c(F)c2[nH]c(C(=O)N3CC(C)(C)C3)cc12]

LBM is selected from any of those in Table A below, and L is selected from any of those in Table B below.

In some embodiments, a provided compound or pharmaceutically acceptable salt thereof, is selected from those wherein SBM is

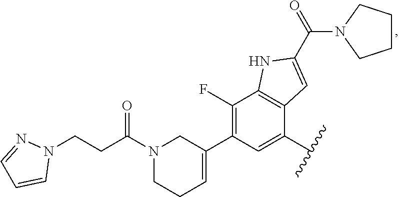 [SMILES: *c1cc(C2=CCCN(C(=O)CCn3cccn3)C2)c(F)c2[nH]c(C(=O)N3CCCC3)cc12]

LBM is selected from any of those in Table A below, and L is selected from any of those in Table B below.

In some embodiments, a provided compound or pharmaceutically acceptable salt thereof, is selected from those wherein SBM is

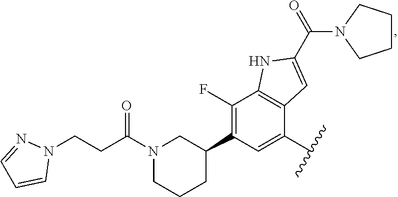 [SMILES: *c1cc([C@H]2CCCN(C(=O)CCn3cccn3)C2)c(F)c2[nH]c(C(=O)N3CCCC3)cc12]

LBM is selected from any of those in Table A below, and L is selected from any of those in Table B below.

In some embodiments, a provided compound or pharmaceutically acceptable salt thereof, is selected from those wherein SBM is

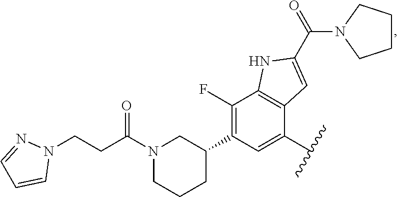 [SMILES: *c1cc([C@@H]2CCCN(C(=O)CCn3cccn3)C2)c(F)c2[nH]c(C(=O)N3CCCC3)cc12]

LBM is selected from any of those in Table A below, and L is selected from any of those in Table B below.

In some embodiments, a provided compound or pharmaceutically acceptable salt thereof, is selected from those wherein SBM is

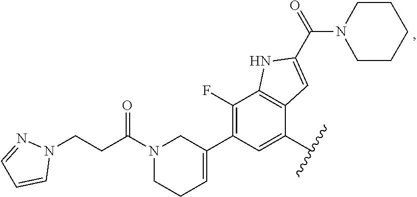 [SMILES: *c1cc(C2=CCCN(C(=O)CCn3cccn3)C2)c(F)c2[nH]c(C(=O)N3CCCCC3)cc12]

LBM is selected from any of those in Table A below, and L is selected from any of those in Table B below.

In some embodiments, a provided compound or pharmaceutically acceptable salt thereof, is selected from those wherein SBM is

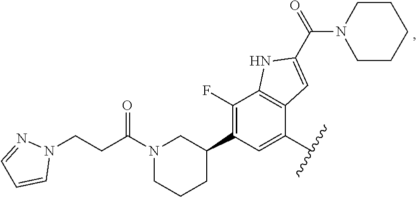 [SMILES: *c1cc([C@H]2CCCN(C(=O)CCn3cccn3)C2)c(F)c2[nH]c(C(=O)N3CCCCC3)cc12]

LBM is selected from any of those in Table A below, and L is selected from any of those in Table B below.

In some embodiments, a provided compound or pharmaceutically acceptable salt thereof, is selected from those wherein SBM is

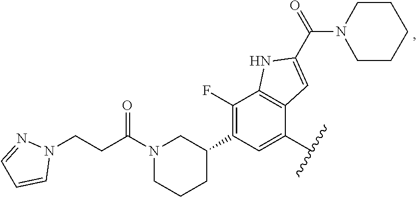 [SMILES: *c1cc([C@@H]2CCCN(C(=O)CCn3cccn3)C2)c(F)c2[nH]c(C(=O)N3CCCCC3)cc12]

LBM is selected from any of those in Table A below, and L is selected from any of those in Table B below.

In some embodiments, a provided compound or pharmaceutically acceptable salt thereof, is selected from those wherein SBM is

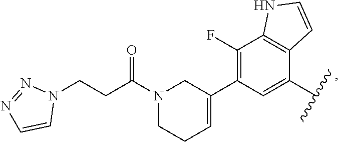 [SMILES: *c1cc(C2=CCCN(C(=O)CCn3ccnn3)C2)c(F)c2[nH]ccc12]

LBM is selected from any of those in Table A below, and L is selected from any of those in Table B below.

In some embodiments, a provided compound or pharmaceutically acceptable salt thereof, is selected from those wherein SBM is

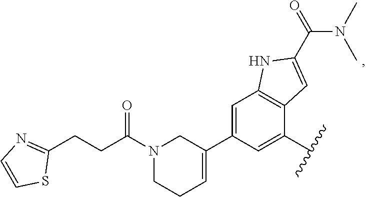 [SMILES: *c1cc(C2=CCCN(C(=O)CCc3nccs3)C2)cc2[nH]c(C(=O)N(C)C)cc12]

LBM is selected from any of those in Table A below, and L is selected from any of those in Table B below.

In some embodiments, a provided compound or pharmaceutically acceptable salt thereof, is selected from those wherein SBM is

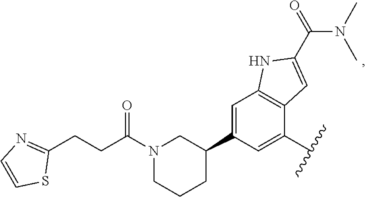 [SMILES: *c1cc([C@H]2CCCN(C(=O)CCc3nccs3)C2)cc2[nH]c(C(=O)N(C)C)cc12]

LBM is selected from any of those in Table A below, and L is selected from any of those in Table B below.

In some embodiments, a provided compound or pharmaceutically acceptable salt thereof, is selected from those wherein SBM is

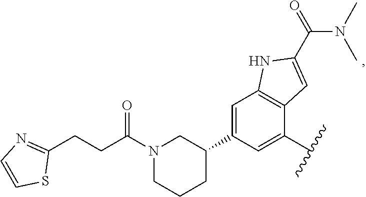 [SMILES: *c1cc([C@@H]2CCCN(C(=O)CCc3nccs3)C2)cc2[nH]c(C(=O)N(C)C)cc12]

LBM is selected from any of those in Table A below, and L is selected from any of those in Table B below.

In some embodiments, a provided compound or pharmaceutically acceptable salt thereof, is selected from those wherein SBM is

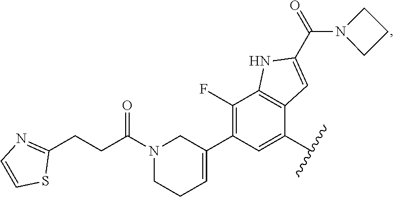 [SMILES: *c1cc(C2=CCCN(C(=O)CCc3nccs3)C2)c(F)c2[nH]c(C(=O)N3CCC3)cc12]

LBM is selected from any of those in Table A below, and L is selected from any of those in Table B below.

In some embodiments, a provided compound or pharmaceutically acceptable salt thereof, is selected from those wherein SBM is

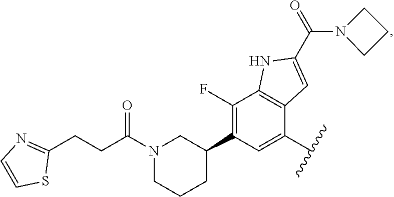 [SMILES: *c1cc([C@H]2CCCN(C(=O)CCc3nccs3)C2)c(F)c2[nH]c(C(=O)N3CCC3)cc12]

LBM is selected from any of those in Table A below, and L is selected from any of those in Table B below.

In some embodiments, a provided compound or pharmaceutically acceptable salt thereof, is selected from those wherein SBM is

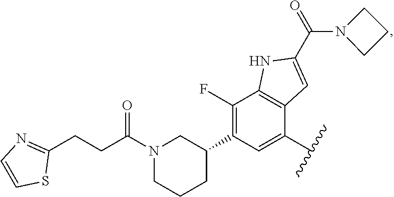 [SMILES: *c1cc([C@@H]2CCCN(C(=O)CCc3nccs3)C2)c(F)c2[nH]c(C(=O)N3CCC3)cc12]

LBM is selected from any of those in Table A below, and L is selected from any of those in Table B below.

In some embodiments, a provided compound or pharmaceutically acceptable salt thereof, is selected from those wherein SBM is

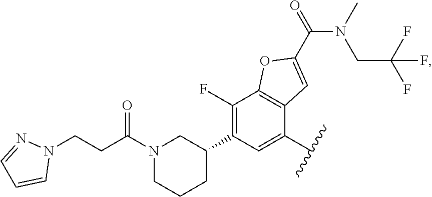 [SMILES: *c1cc([C@@H]2CCCN(C(=O)CCn3cccn3)C2)c(F)c2oc(C(=O)N(C)CC(F)(F)F)cc12]

LBM is selected from any of those in Table A below, and L is selected from any of those in Table B below.

In some embodiments, a provided compound or pharmaceutically acceptable salt thereof, is selected from those wherein SBM is

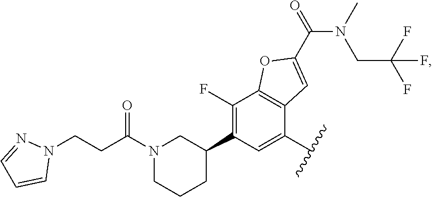 [SMILES: *c1cc([C@H]2CCCN(C(=O)CCn3cccn3)C2)c(F)c2oc(C(=O)N(C)CC(F)(F)F)cc12]

LBM is selected from any of those in Table A below, and L is selected from any of those in Table B below.

In some embodiments, a provided compound or pharmaceutically acceptable salt thereof, is selected from those wherein SBM is

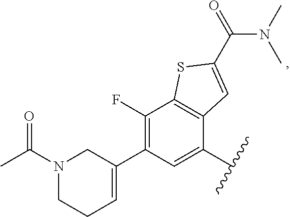 [SMILES: *c1cc(C2=CCCN(C(C)=O)C2)c(F)c2sc(C(=O)N(C)C)cc12]

LBM is selected from any of those in Table A below, and L is selected from any of those in Table B below.

In some embodiments, a provided compound or pharmaceutically acceptable salt thereof, is selected from those wherein SBM is

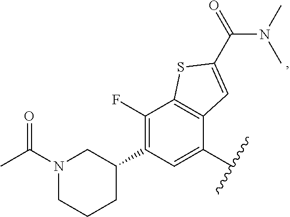 [SMILES: *c1cc([C@@H]2CCCN(C(C)=O)C2)c(F)c2sc(C(=O)N(C)C)cc12]

LBM is selected from any of those in Table A below, and L is selected from any of those in Table B below.

In some embodiments, a provided compound or pharmaceutically acceptable salt thereof, is selected from those wherein SBM is

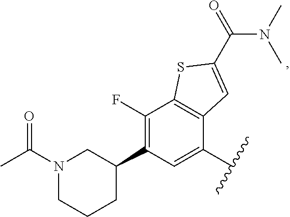 [SMILES: *c1cc([C@H]2CCCN(C(C)=O)C2)c(F)c2sc(C(=O)N(C)C)cc12]

LBM is selected from any of those in Table A below, and L is selected from any of those in Table B below.

In some embodiments, a provided compound or pharmaceutically acceptable salt thereof, is selected from those wherein SBM is

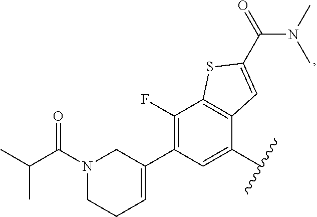 [SMILES: *c1cc(C2=CCCN(C(=O)C(C)C)C2)c(F)c2sc(C(=O)N(C)C)cc12]

LBM is selected from any of those in Table A below, and L is selected from any of those in Table B below.

In some embodiments, a provided compound or pharmaceutically acceptable salt thereof, is selected from those wherein SBM is

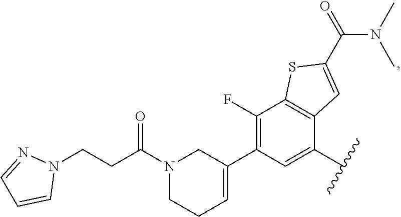 [SMILES: *c1cc(C2=CCCN(C(=O)CCn3cccn3)C2)c(F)c2sc(C(=O)N(C)C)cc12]

LBM is selected from any of those in Table A below, and L is selected from any of those in Table B below.

In some embodiments, a provided compound or pharmaceutically acceptable salt thereof, is selected from those wherein SBM is

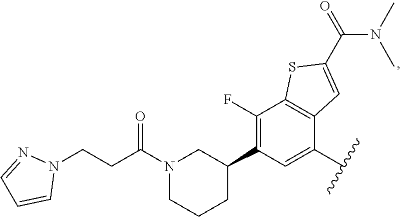 [SMILES: *c1cc([C@H]2CCCN(C(=O)CCn3cccn3)C2)c(F)c2sc(C(=O)N(C)C)cc12]

LBM is selected from any of those in Table A below, and L is selected from any of those in Table B below.

In some embodiments, a provided compound or pharmaceutically acceptable salt thereof, is selected from those wherein SBM is

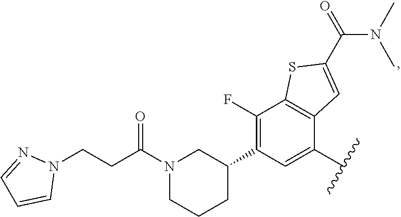 [SMILES: *c1cc([C@@H]2CCCN(C(=O)CCn3cccn3)C2)c(F)c2sc(C(=O)N(C)C)cc12]

LBM is selected from any of those in Table A below, and L is selected from any of those in Table B below.

In some embodiments, a provided compound or pharmaceutically acceptable salt thereof, is selected from those wherein SBM is

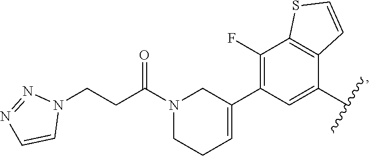 [SMILES: *c1cc(C2=CCCN(C(=O)CCn3ccnn3)C2)c(F)c2sccc12]

LBM is selected from any of those in Table A below, and L is selected from any of those in Table B below.

In some embodiments, a provided compound or pharmaceutically acceptable salt thereof, is selected from those wherein SBM is

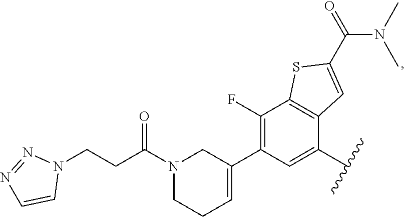 [SMILES: *c1cc(C2=CCCN(C(=O)CCn3ccnn3)C2)c(F)c2sc(C(=O)N(C)C)cc12]

LBM is selected from any of those in Table A below, and L is selected from any of those in Table B below.

In some embodiments, a provided compound or pharmaceutically acceptable salt thereof, is selected from those wherein SBM is

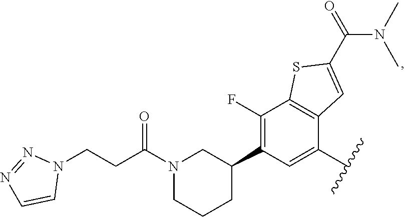 [SMILES: *c1cc([C@H]2CCCN(C(=O)CCn3ccnn3)C2)c(F)c2sc(C(=O)N(C)C)cc12]

LBM is selected from any of those in Table A below, and L is selected from any of those in Table B below.

In some embodiments, a provided compound or pharmaceutically acceptable salt thereof, is selected from those wherein SBM is

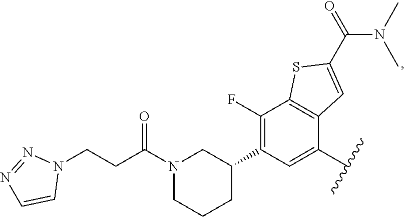 [SMILES: *c1cc([C@@H]2CCCN(C(=O)CCn3ccnn3)C2)c(F)c2sc(C(=O)N(C)C)cc12]

LBM is selected from any of those in Table A below, and L is selected from any of those in Table B below.

In some embodiments, a provided compound or pharmaceutically acceptable salt thereof, is selected from those wherein SBM is

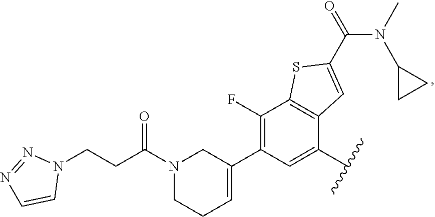 [SMILES: *c1cc(C2=CCCN(C(=O)CCn3ccnn3)C2)c(F)c2sc(C(=O)N(C)C3CC3)cc12]

LBM is selected from any of those in Table A below, and L is selected from any of those in Table B below.

In some embodiments, a provided compound or pharmaceutically acceptable salt thereof, is selected from those wherein SBM is

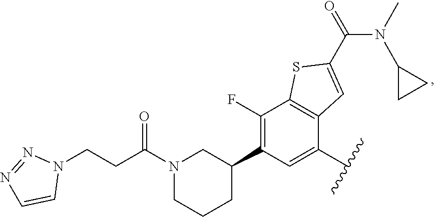 [SMILES: *c1cc([C@H]2CCCN(C(=O)CCn3ccnn3)C2)c(F)c2sc(C(=O)N(C)C3CC3)cc12]

LBM is selected from any of those in Table A below, and L is selected from any of those in Table B below.

In some embodiments, a provided compound or pharmaceutically acceptable salt thereof, is selected from those wherein SBM is

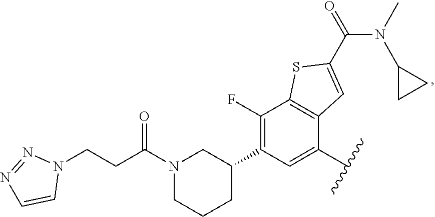 [SMILES: *c1cc([C@@H]2CCCN(C(=O)CCn3ccnn3)C2)c(F)c2sc(C(=O)N(C)C3CC3)cc12]

LBM is selected from any of those in Table A below, and L is selected from any of those in Table B below.

In some embodiments, a provided compound or pharmaceutically acceptable salt thereof, is selected from those wherein SBM is

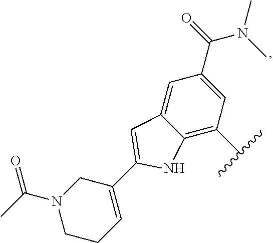 [SMILES: *c1cc(C(=O)N(C)C)cc2cc(C3=CCCN(C(C)=O)C3)[nH]c12]

LBM is selected from any of those in Table A below, and L is selected from any of those in Table B below.

In some embodiments, a provided compound or pharmaceutically acceptable salt thereof, is selected from those wherein SBM is

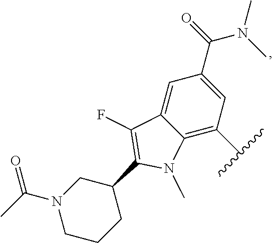 [SMILES: *c1cc(C(=O)N(C)C)cc2c(F)c([C@H]3CCCN(C(C)=O)C3)n(C)c12]

LBM is selected from any of those in Table A below, and L is selected from any of those in Table B below.

In some embodiments, a provided compound or pharmaceutically acceptable salt thereof, is selected from those wherein SBM is

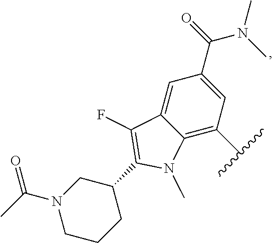 [SMILES: *c1cc(C(=O)N(C)C)cc2c(F)c([C@@H]3CCCN(C(C)=O)C3)n(C)c12]

LBM is selected from any of those in Table A below, and L is selected from any of those in Table B below.

In some embodiments, a provided compound or pharmaceutically acceptable salt thereof, is selected from those wherein SBM is

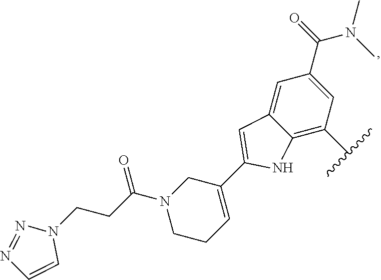 [SMILES: *c1cc(C(=O)N(C)C)cc2cc(C3=CCCN(C(=O)CCn4ccnn4)C3)[nH]c12]

LBM is selected from any of those in Table A below, and L is selected from any of those in Table B below.

In some embodiments, a provided compound or pharmaceutically acceptable salt thereof, is selected from those wherein SBM is

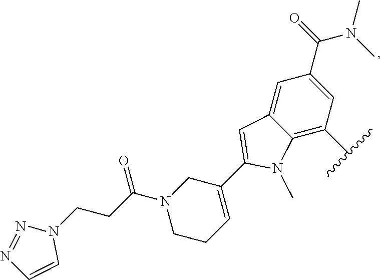 [SMILES: *c1cc(C(=O)N(C)C)cc2cc(C3=CCCN(C(=O)CCn4ccnn4)C3)n(C)c12]

LBM is selected from any of those in Table A below, and L is selected from any of those in Table B below.

In some embodiments, a provided compound or pharmaceutically acceptable salt thereof, is selected from those wherein SBM is

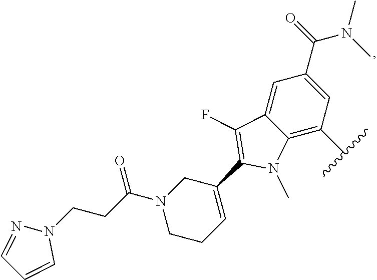

LBM is selected from any of those in Table A below, and L is selected from any of those in Table B below.

In some embodiments, a provided compound or pharmaceutically acceptable salt thereof, is selected from those wherein SBM is

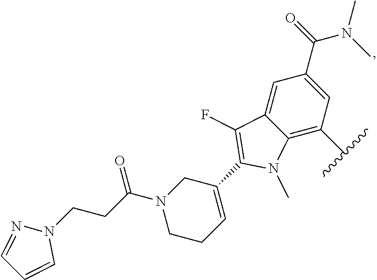

LBM is selected from any of those in Table A below, and L is selected from any of those in Table B below.

In some embodiments, a provided compound or pharmaceutically acceptable salt thereof, is selected from those wherein SBM is

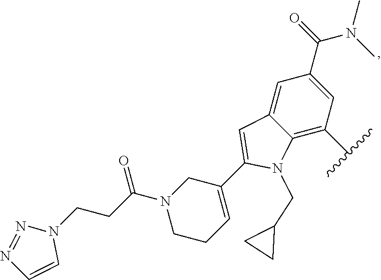 [SMILES: *c1cc(C(=O)N(C)C)cc2cc(C3=CCCN(C(=O)CCn4ccnn4)C3)n(CC3CC3)c12]

LBM is selected from any of those in Table A below, and L is selected from any of those in Table B below.

In some embodiments, a provided compound or pharmaceutically acceptable salt thereof, is selected from those wherein SBM is

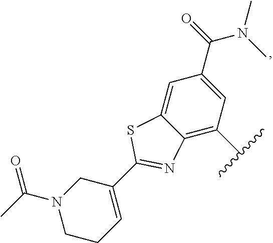 [SMILES: *c1cc(C(=O)N(C)C)cc2sc(C3=CCCN(C(C)=O)C3)nc12]

LBM is selected from any of those in Table A below, and L is selected from any of those in Table B below.

In some embodiments, a provided compound or pharmaceutically acceptable salt thereof, is selected from those wherein SBM is

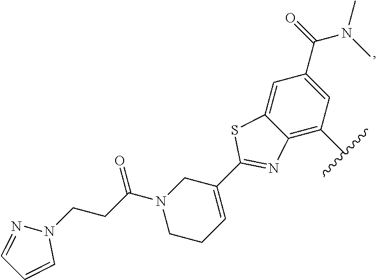 [SMILES: *c1cc(C(=O)N(C)C)cc2sc(C3=CCCN(C(=O)CCn4cccn4)C3)nc12]

LBM is selected from any of those in Table A below, and L is selected from any of those in Table B below.

In some embodiments, a provided compound or pharmaceutically acceptable salt thereof, is selected from those wherein SBM is

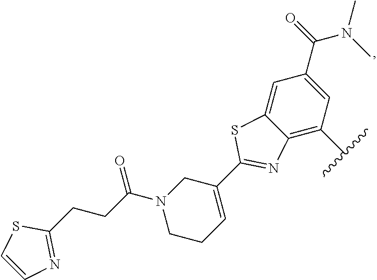 [SMILES: *c1cc(C(=O)N(C)C)cc2sc(C3=CCCN(C(=O)CCc4nccs4)C3)nc12]

LBM is selected from any of those in Table A below, and L is selected from any of those in Table B below.

In some embodiments, a provided compound or pharmaceutically acceptable salt thereof, is selected from those wherein SBM is

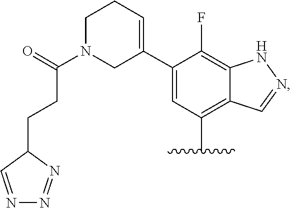 [SMILES: *c1cc(C2=CCCN(C(=O)CCC3C=NN=N3)C2)c(F)c2[nH]ncc12]

LBM is selected from any of those in Table A below, and L is selected from any of those in Table B below.

In some embodiments, a provided compound or pharmaceutically acceptable salt thereof, is selected from those wherein SBM is

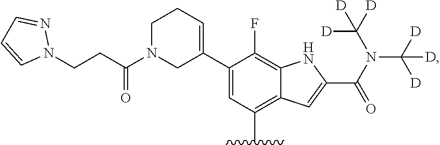

LBM is selected from any of those in Table A below, and L is selected from any of those in Table B below.

In some embodiments, a provided compound or pharmaceutically acceptable salt thereof, is selected from those wherein SBM is

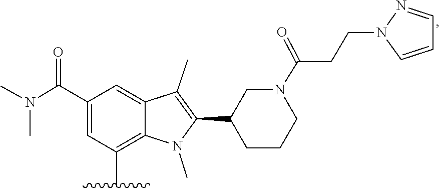

LBM is selected from any of those in Table A below, and L is selected from any of those in Table B below.

In some embodiments, a provided compound or pharmaceutically acceptable salt thereof, is selected from those wherein SBM is

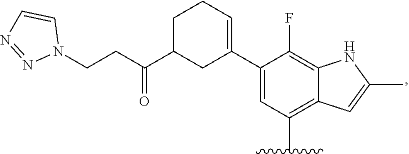 [SMILES: *c1cc(C2=CCCC(C(=O)CCn3ccnn3)C2)c(F)c2[nH]c(C)cc12]

LBM is selected from any of those in Table A below, and L is selected from any of those in Table B below.

In some embodiments, a provided compound or pharmaceutically acceptable salt thereof, is selected from those wherein SBM is

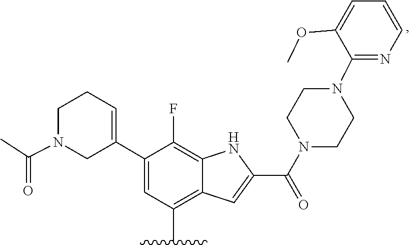 [SMILES: *c1cc(C2=CCCN(C(C)=O)C2)c(F)c2[nH]c(C(=O)N3CCN(c4ncccc4OC)CC3)cc12]

LBM is selected from any of those in Table A below, and L is selected from any of those in Table B below.

TABLE A
Exemplified E3 Ligase Binding Moiety (LBM)

| | |
|---|---|
| 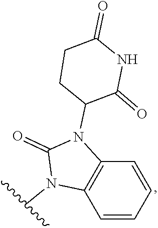 [SMILES: *n1c(=O)n(C2CCC(=O)NC2=O)c2ccccc21] | (e) |
| 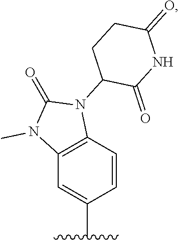 [SMILES: *c1ccc2c(c1)n(C)c(=O)n2C1CCC(=O)NC1=O] | (f) |
| 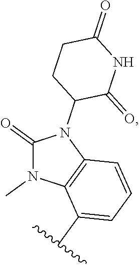 [SMILES: *c1cccc2c1n(C)c(=O)n2C1CCC(=O)NC1=O] | (g) |
| 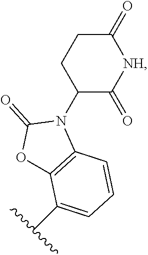 [SMILES: *c1cccc2c1oc(=O)n2C1CCC(=O)NC1=O] | (h) |
| 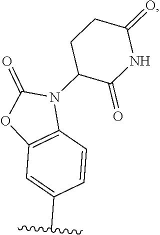 [SMILES: *c1ccc2c(c1)oc(=O)n2C1CCC(=O)NC1=O] | (i) |
| 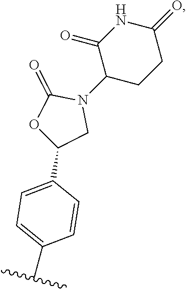 [SMILES: *c1ccc([C@H]2CN(C3CCC(=O)NC3=O)C(=O)O2)cc1] | (j) |
| 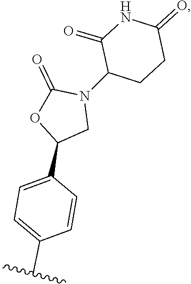 [SMILES: *c1ccc([C@@H]2CN(C3CCC(=O)NC3=O)C(=O)O2)cc1] | (k) |
| 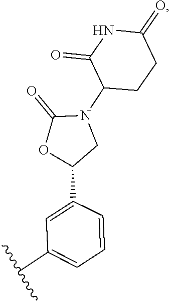 [SMILES: *c1cccc([C@H]2CN(C3CCC(=O)NC3=O)C(=O)O2)c1] | (l) |
| 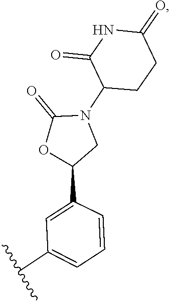 [SMILES: *c1cccc([C@@H]2CN(C3CCC(=O)NC3=O)C(=O)O2)c1] | (m) |
| 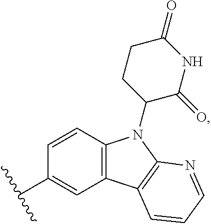 [SMILES: *c1ccc2c(c1)c1cccnc1n2C1CCC(=O)NC1=O] | (n) |
| 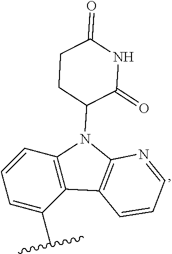 [SMILES: *c1cccc2c1c1cccnc1n2C1CCC(=O)NC1=O] | (o) |
| 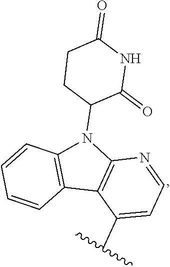 [SMILES: *c1ccnc2c1c1ccccc1n2C1CCC(=O)NC1=O] | (p) |
| 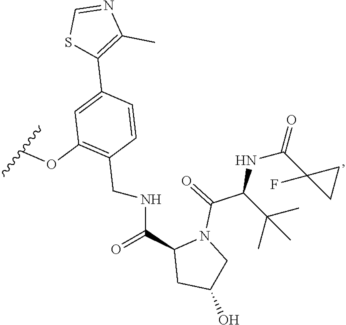 [SMILES: *Oc1cc(-c2scnc2C)ccc1CNC(=O)[C@@H]1C[C@@H](O)CN1C(=O)[C@@H](NC(=O)C1(F)CC1)C(C)(C)C] | (n) |
| 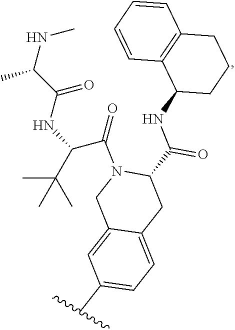 [SMILES: *c1ccc2c(c1)CN(C(=O)[C@@H](NC(=O)[C@H](C)NC)C(C)(C)C)[C@H](C(=O)N[C@@H]1CCCc3ccccc31)C2] | (o) |
| 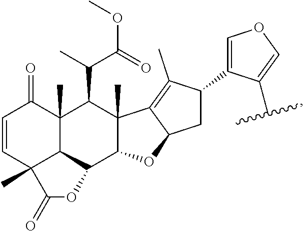 [SMILES: *c1cocc1[C@@H]1C[C@H]2O[C@@H]3[C@@H]4OC(=O)[C@]5(C)C=CC(=O)[C@](C)([C@@H](C(C)C(=O)OC)[C@]3(C)C2=C1C)[C@@H]45] | (p) |
| 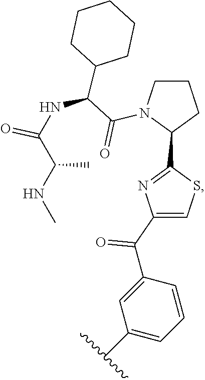 [SMILES: *c1cccc(C(=O)c2csc([C@@H]3CCCN3C(=O)[C@@H](NC(=O)[C@H](C)NC)C3CCCCC3)n2)c1] | (q) |
| 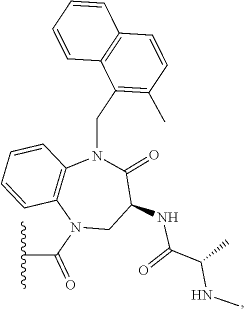 [SMILES: *C(=O)N1C[C@H](NC(=O)[C@H](C)NC)C(=O)N(Cc2c(C)ccc3ccccc23)c2ccccc21] | (r) |
| 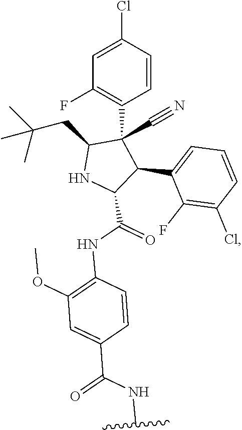 [SMILES: *NC(=O)c1ccc(NC(=O)[C@@H]2N[C@@H](CC(C)(C)C)[C@](C#N)(c3ccc(Cl)cc3F)[C@H]2c2cccc(Cl)c2F)c(OC)c1] | (s) |
| 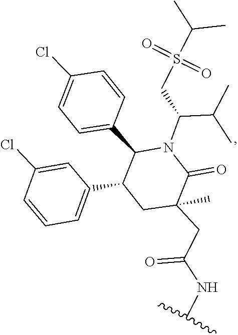 [SMILES: *NC(=O)C[C@@]1(C)C[C@H](c2cccc(Cl)c2)[C@@H](c2ccc(Cl)cc2)N([C@H](CS(=O)(=O)C(C)C)C(C)C)C1=O] | (t) |
| 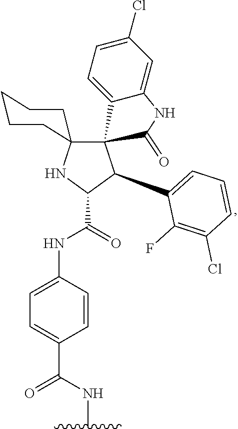 [SMILES: *NC(=O)c1ccc(NC(=O)[C@@H]2NC3(CCCCC3)[C@@]3(C(=O)Nc4cc(Cl)ccc43)[C@H]2c2cccc(Cl)c2F)cc1] | (u) |
| 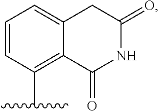 [SMILES: *c1cccc2c1C(=O)NC(=O)C2] | (v) |
| 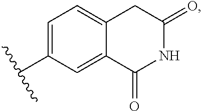 [SMILES: *c1ccc2c(c1)C(=O)NC(=O)C2] | (w) |
| 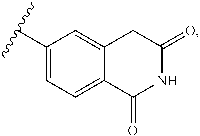 [SMILES: *c1ccc2c(c1)CC(=O)NC2=O] | (x) |
| 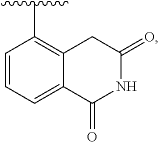 [SMILES: *c1cccc2c1CC(=O)NC2=O] | (y) |
| 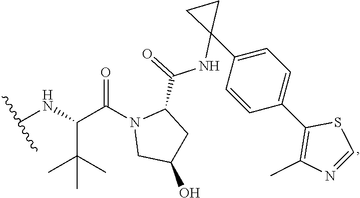 [SMILES: *N[C@H](C(=O)N1C[C@H](O)C[C@H]1C(=O)NC1(c2ccc(-c3scnc3C)cc2)CC1)C(C)(C)C] | (z) |
| 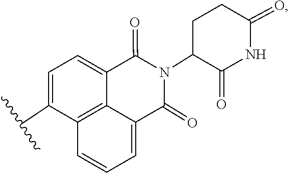 [SMILES: *c1ccc2c3c(cccc13)C(=O)N(C1CCC(=O)NC1=O)C2=O] | (bb) |
| 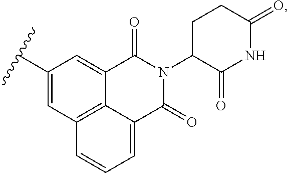 [SMILES: *c1cc2c3c(cccc3c1)C(=O)N(C1CCC(=O)NC1=O)C2=O] | (cc) |
| 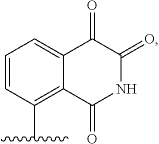 [SMILES: *c1cccc2c1C(=O)NC(=O)C2=O] | (dd) |
| 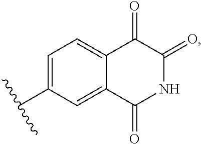 [SMILES: *c1ccc2c(c1)C(=O)NC(=O)C2=O] | (ee) |
| 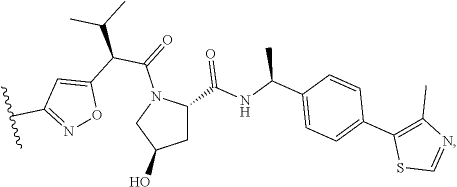 [SMILES: *c1cc([C@H](C(=O)N2C[C@H](O)C[C@H]2C(=O)N[C@@H](C)c2ccc(-c3scnc3C)cc2)C(C)C)on1] | (ff) |
| 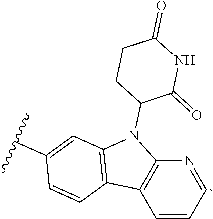 [SMILES: *c1ccc2c3cccnc3n(C3CCC(=O)NC3=O)c2c1] | (gg) |
| 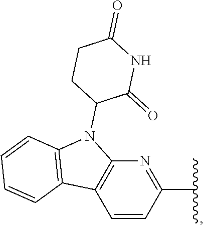 [SMILES: *c1ccc2c3ccccc3n(C3CCC(=O)NC3=O)c2n1] | (hh) |
| 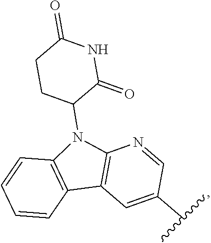 [SMILES: *c1cnc2c(c1)c1ccccc1n2C1CCC(=O)NC1=O] | (ii) |
| 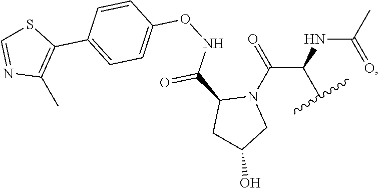 [SMILES: *[C@H](NC(C)=O)C(=O)N1C[C@H](O)C[C@H]1C(=O)NOc1ccc(-c2scnc2C)cc1] | (jj) |
| 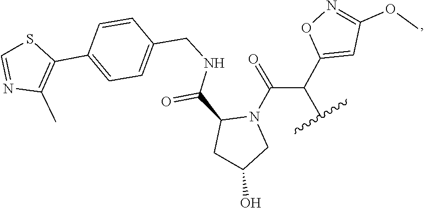 [SMILES: *C(C(=O)N1C[C@H](O)C[C@H]1C(=O)NCc1ccc(-c2scnc2C)cc1)c1cc(OC)no1] | (kk) |
| 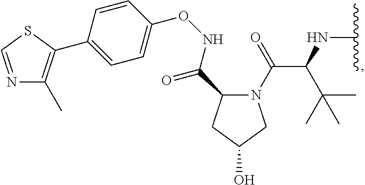 [SMILES: *N[C@H](C(=O)N1C[C@H](O)C[C@H]1C(=O)NOc1ccc(-c2scnc2C)cc1)C(C)(C)C] | (ll) |
| 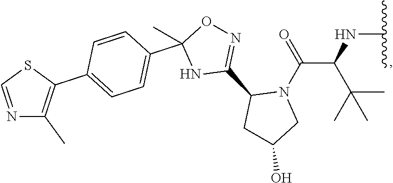 [SMILES: *N[C@H](C(=O)N1C[C@H](O)C[C@H]1C1=NOC(C)(c2ccc(-c3scnc3C)cc2)N1)C(C)(C)C] | (mm) |
| 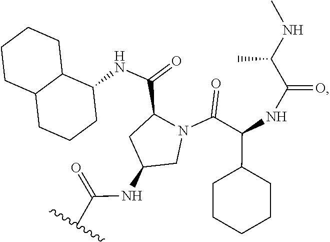 [SMILES: *C(=O)N[C@H]1C[C@@H](C(=O)N[C@@H]2CCCC3CCCCC32)N(C(=O)[C@@H](NC(=O)[C@H](C)NC)C2CCCCC2)C1] | (nn) |
| 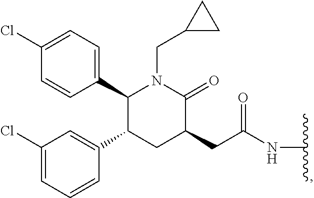 [SMILES: *NC(=O)C[C@H]1C[C@H](c2cccc(Cl)c2)[C@@H](c2ccc(Cl)cc2)N(CC2CC2)C1=O] | (oo) |
| 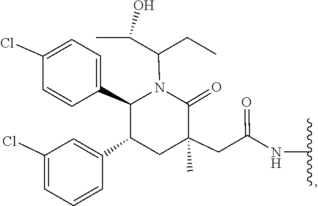 [SMILES: *NC(=O)C[C@@]1(C)C[C@H](c2cccc(Cl)c2)[C@@H](c2ccc(Cl)cc2)N(C(CC)[C@H](C)O)C1=O] | (pp) |
| 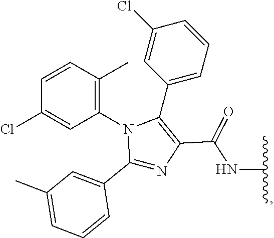 [SMILES: *NC(=O)c1nc(-c2cccc(C)c2)n(-c2cc(Cl)ccc2C)c1-c1cccc(Cl)c1] | (qq) |
| 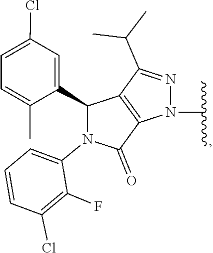 [SMILES: *n1nc(C(C)C)c2c1C(=O)N(c1cccc(Cl)c1F)[C@H]2c1cc(Cl)ccc1C] | (rr) |
| 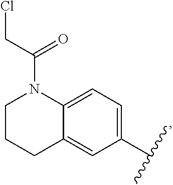 [SMILES: *c1ccc2c(c1)CCCN2C(=O)CCl] | (ss) |
| 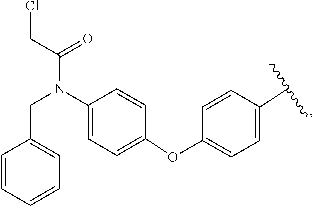 [SMILES: *c1ccc(Oc2ccc(N(Cc3ccccc3)C(=O)CCl)cc2)cc1] | (tt) |
| 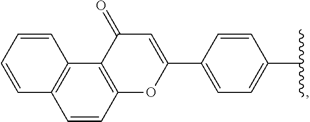 [SMILES: *c1ccc(-c2cc(=O)c3c(ccc4ccccc43)o2)cc1] | (uu) |
| 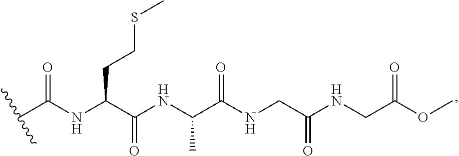 [SMILES: *C(=O)N[C@@H](CCSC)C(=O)N[C@@H](C)C(=O)NCC(=O)NCC(=O)OC] | (vv) |
| 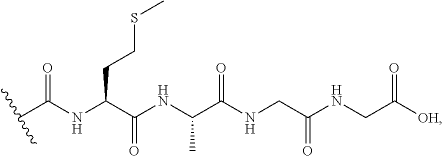 [SMILES: *C(=O)N[C@@H](CCSC)C(=O)N[C@@H](C)C(=O)NCC(=O)NCC(=O)O] | (ww) |
| 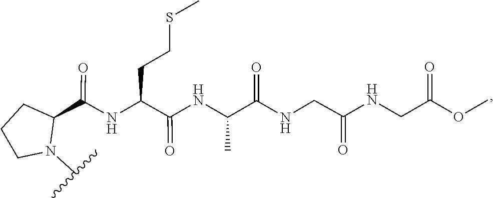 [SMILES: *N1CCC[C@H]1C(=O)N[C@@H](CCSC)C(=O)N[C@@H](C)C(=O)NCC(=O)NCC(=O)OC] | (xx) |
| 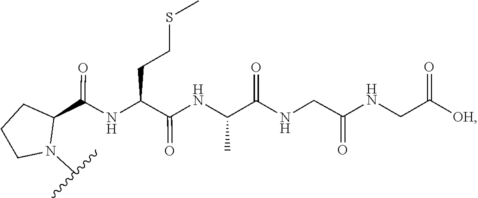 [SMILES: *N1CCC[C@H]1C(=O)N[C@@H](CCSC)C(=O)N[C@@H](C)C(=O)NCC(=O)NCC(=O)O] | (yy) |
| 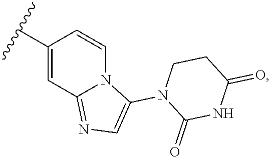 [SMILES: *c1ccn2c(N3CCC(=O)NC3=O)cnc2c1] | (zz) |
| 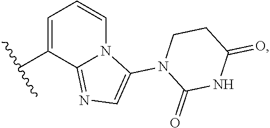 [SMILES: *c1cccn2c(N3CCC(=O)NC3=O)cnc12] | (aaa) |
| 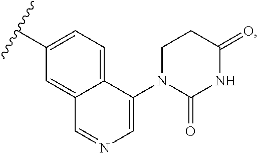 [SMILES: *c1ccc2c(N3CCC(=O)NC3=O)cncc2c1] | (bbb) |
| 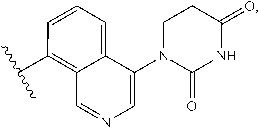 [SMILES: *c1cccc2c(N3CCC(=O)NC3=O)cncc12] | (ccc) |
| 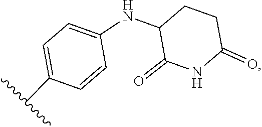 [SMILES: *c1ccc(NC2CCC(=O)NC2=O)cc1] | (ddd) |
| 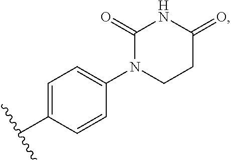 [SMILES: *c1ccc(N2CCC(=O)NC2=O)cc1] | (eee) |
| 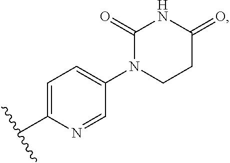 [SMILES: *c1ccc(N2CCC(=O)NC2=O)cn1] | (fff) |
| 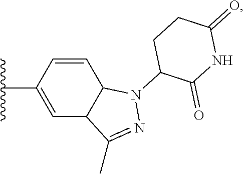 [SMILES: *C1=CC2C(C)=NN(C3CCC(=O)NC3=O)C2C=C1] | (ggg) |
| 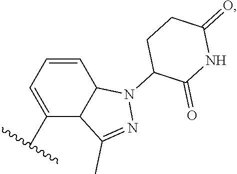 [SMILES: *C1=CC=CC2C1C(C)=NN2C1CCC(=O)NC1=O] | (hhh) |
| 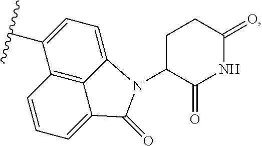 [SMILES: *c1ccc2c3c(cccc13)C(=O)N2C1CCC(=O)NC1=O] | (iii) |
| 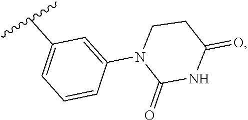 [SMILES: *c1cccc(N2CCC(=O)NC2=O)c1] | (jjj) |
| 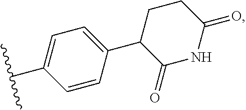 [SMILES: *c1ccc(C2CCC(=O)NC2=O)cc1] | (kkk) |
| 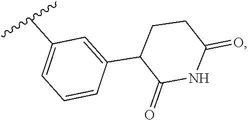 [SMILES: *c1cccc(C2CCC(=O)NC2=O)c1] | (lll) |
| 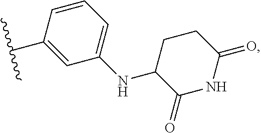 [SMILES: *c1cccc(NC2CCC(=O)NC2=O)c1] | (mmm) |
| 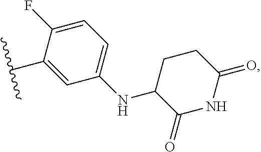 [SMILES: *c1cc(NC2CCC(=O)NC2=O)ccc1F] | (nnn) |
| 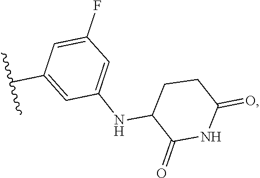 [SMILES: *c1cc(F)cc(NC2CCC(=O)NC2=O)c1] | (ooo) |
| 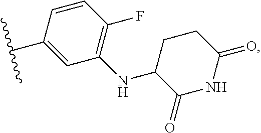 [SMILES: *c1ccc(F)c(NC2CCC(=O)NC2=O)c1] | (ppp) |
| 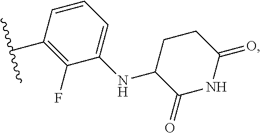 [SMILES: *c1cccc(NC2CCC(=O)NC2=O)c1F] | (qqq) |
| 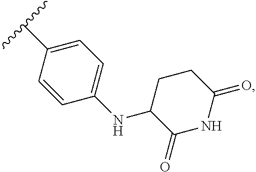 [SMILES: *c1ccc(NC2CCC(=O)NC2=O)cc1] | (rrr) |
| 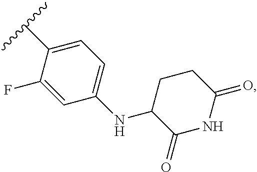 [SMILES: *c1ccc(NC2CCC(=O)NC2=O)cc1F] | (sss) |
| 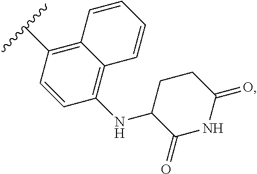 [SMILES: *c1ccc(NC2CCC(=O)NC2=O)c2ccccc12] | (ttt) |
| 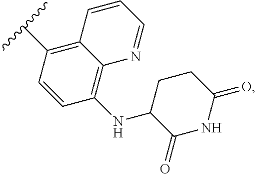 [SMILES: *c1ccc(NC2CCC(=O)NC2=O)c2ncccc12] | (uuu) |
| 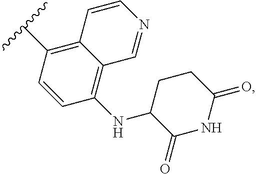 [SMILES: *c1ccc(NC2CCC(=O)NC2=O)c2cnccc12] | (vvv) |
| 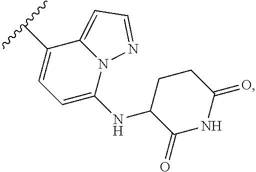 [SMILES: *c1ccc(NC2CCC(=O)NC2=O)n2nccc12] | (www) |
| 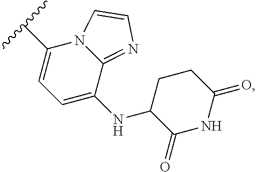 [SMILES: *c1ccc(NC2CCC(=O)NC2=O)c2nccn12] | (xxx) |
| 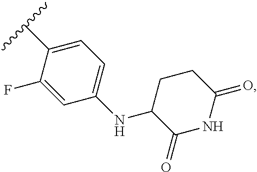 [SMILES: *c1ccc(NC2CCC(=O)NC2=O)cc1F] | (yyy) |
| 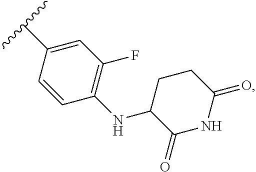 [SMILES: *c1ccc(NC2CCC(=O)NC2=O)c(F)c1] | (zzz) |
| 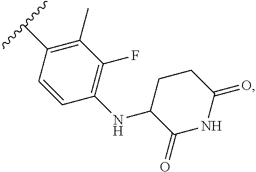 [SMILES: *c1ccc(NC2CCC(=O)NC2=O)c(F)c1C] | (aaaa) |
| 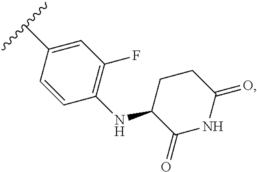 [SMILES: *c1ccc(N[C@H]2CCC(=O)NC2=O)c(F)c1] | (bbbb) |
| 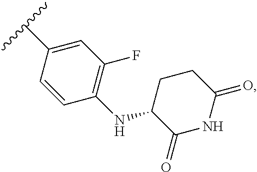 [SMILES: *c1ccc(N[C@@H]2CCC(=O)NC2=O)c(F)c1] | (cccc) |
| 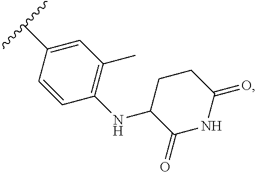 [SMILES: *c1ccc(NC2CCC(=O)NC2=O)c(C)c1] | (dddd) |
| 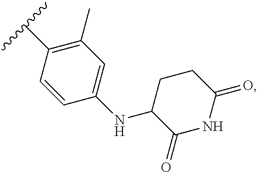 [SMILES: *c1ccc(NC2CCC(=O)NC2=O)cc1C] | (eeee) |
| 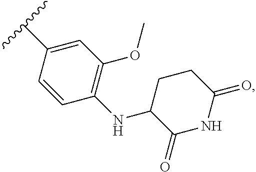 [SMILES: *c1ccc(NC2CCC(=O)NC2=O)c(OC)c1] | (ffff) |
| 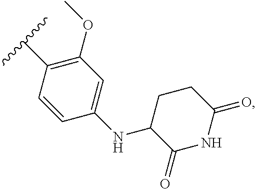 [SMILES: *c1ccc(NC2CCC(=O)NC2=O)cc1OC] | (gggg) |
| 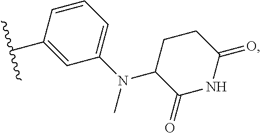 [SMILES: *c1cccc(N(C)C2CCC(=O)NC2=O)c1] | (hhhh) |
| 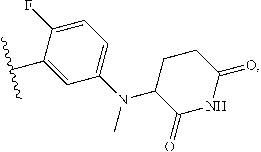 [SMILES: *c1cc(N(C)C2CCC(=O)NC2=O)ccc1F] | (iiii) |
| 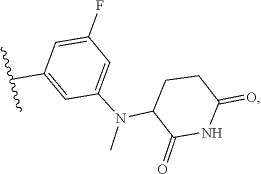 [SMILES: *c1cc(F)cc(N(C)C2CCC(=O)NC2=O)c1] | (jjjj) |
| 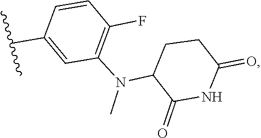 [SMILES: *c1ccc(F)c(N(C)C2CCC(=O)NC2=O)c1] | (kkkk) |
| 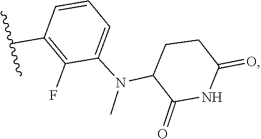 [SMILES: *c1cccc(N(C)C2CCC(=O)NC2=O)c1F] | (llll) |
| 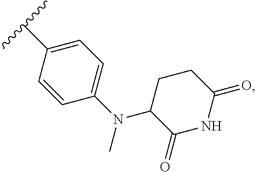 [SMILES: *c1ccc(N(C)C2CCC(=O)NC2=O)cc1] | (mmmm) |
| 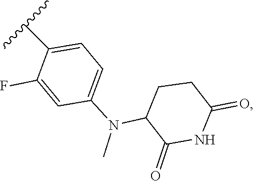 [SMILES: *c1ccc(N(C)C2CCC(=O)NC2=O)cc1F] | (nnnn) |
| 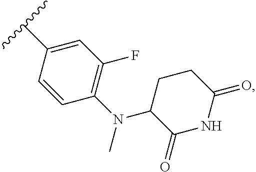 [SMILES: *c1ccc(N(C)C2CCC(=O)NC2=O)c(F)c1] | (oooo) |
| 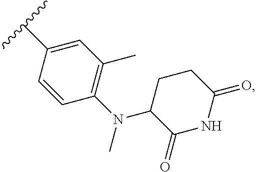 [SMILES: *c1ccc(N(C)C2CCC(=O)NC2=O)c(C)c1] | (pppp) |
| 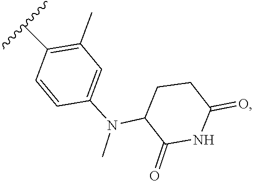 [SMILES: *c1ccc(N(C)C2CCC(=O)NC2=O)cc1C] | (qqqq) |
| 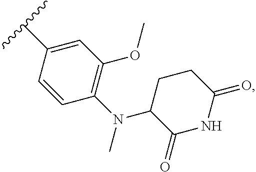 [SMILES: *c1ccc(N(C)C2CCC(=O)NC2=O)c(OC)c1] | (rrrr) |
| 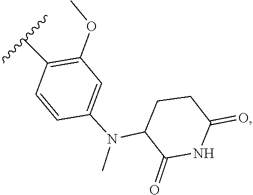 [SMILES: *c1ccc(N(C)C2CCC(=O)NC2=O)cc1OC] | (ssss) |
| 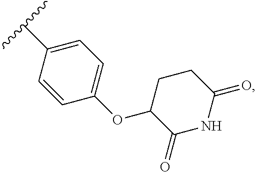 [SMILES: *c1ccc(OC2CCC(=O)NC2=O)cc1] | (tttt) |
| 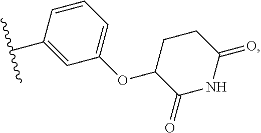 [SMILES: *c1cccc(OC2CCC(=O)NC2=O)c1] | (uuuu) |
| 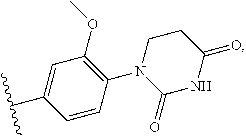 [SMILES: *c1ccc(N2CCC(=O)NC2=O)c(OC)c1] | (vvvv) |
| 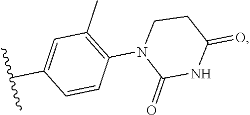 [SMILES: *c1ccc(N2CCC(=O)NC2=O)c(C)c1] | (wwww) |
| 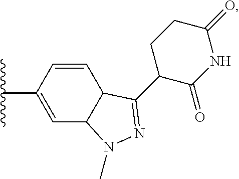 [SMILES: *C1=CC2C(C=C1)C(C1CCC(=O)NC1=O)=NN2C] | (xxxx) |
| 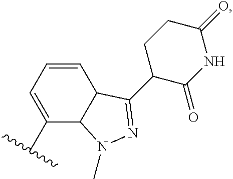 [SMILES: *C1=CC=CC2C(C3CCC(=O)NC3=O)=NN(C)C12] | (yyyy) |
| 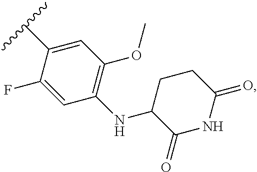 [SMILES: *c1cc(OC)c(NC2CCC(=O)NC2=O)cc1F] | (zzzz) |
| 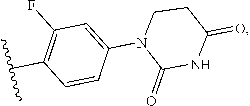 [SMILES: *c1ccc(N2CCC(=O)NC2=O)cc1F] | (aaaaa) |
| 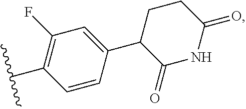 [SMILES: *c1ccc(C2CCC(=O)NC2=O)cc1F] | (bbbbb) |
| 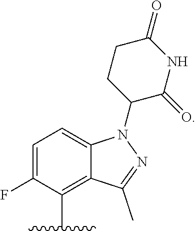 [SMILES: *c1c(F)ccc2c1c(C)nn2C1CCC(=O)NC1=O] | (cccccc) |

or any of the LBM disclosed herein.

In some embodiments, the present invention provides a compound or pharmaceutically acceptable salt thereof having a STAT6 binding moiety and L described and disclosed herein, wherein LBM is selected from any of those in Table A.

TABLE B
Exemplified Linkers (L)

| | |
|---|---|
| 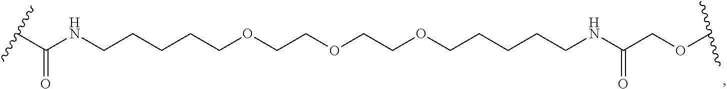 [SMILES: *OCC(=O)NCCCCCOCCOCCOCCCCCNC(*)=O] | (1) |
| 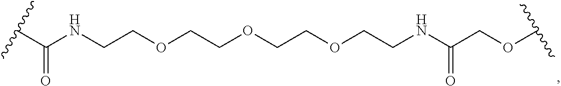 [SMILES: *OCC(=O)NCCOCCOCCOCCNC(*)=O] | (2) |
| 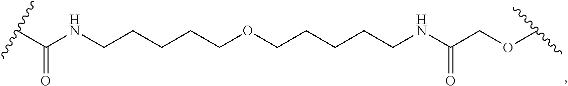 [SMILES: *OCC(=O)NCCCCCOCCCCCNC(*)=O] | (3) |
| 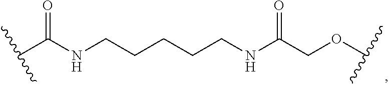 [SMILES: *OCC(=O)NCCCCCNC(*)=O] | (4) |
| 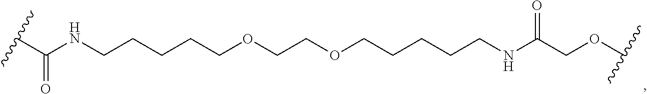 [SMILES: *OCC(=O)NCCCCCOCCOCCCCCNC(*)=O] | (5) |
| 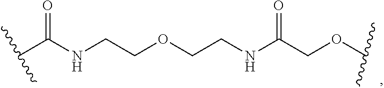 [SMILES: *OCC(=O)NCCOCCNC(*)=O] | (6) |
| 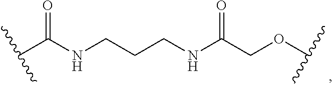 [SMILES: *OCC(=O)NCCCNC(*)=O] | (7) |
| 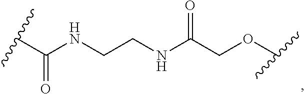 [SMILES: *OCC(=O)NCCNC(*)=O] | (8) |
| 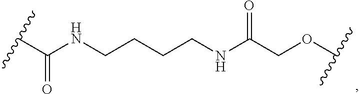 [SMILES: *OCC(=O)NCCCCNC(*)=O] | (9) |
| 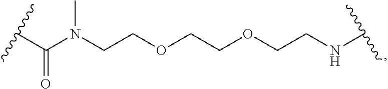 [SMILES: *NCCOCCOCCN(C)C(*)=O] | (10) |
| 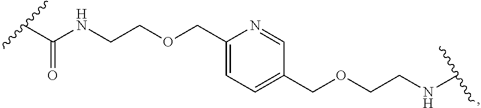 [SMILES: *NCCOCc1ccc(COCCNC(*)=O)nc1] | (11) |
| 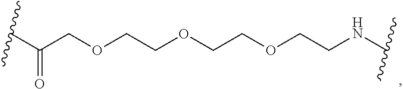 [SMILES: *NCCOCCOCCOCC(*)=O] | (12) |
| 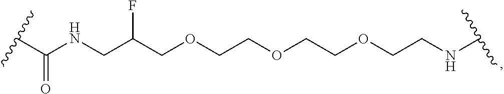 [SMILES: *NCCOCCOCCOCC(F)CNC(*)=O] | (13) |
| 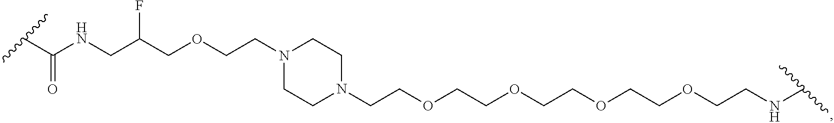 [SMILES: *NCCOCCOCCOCCOCCN1CCN(CCOCC(F)CNC(*)=O)CC1] | (14) |
| 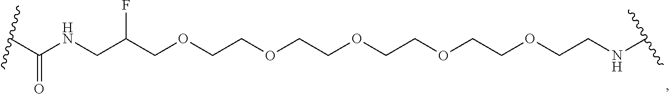 [SMILES: *NCCOCCOCCOCCOCCOCC(F)CNC(*)=O] | (15) |
| 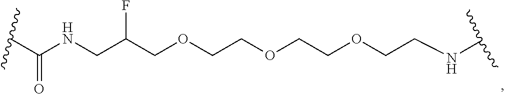 [SMILES: *NCCOCCOCCOCC(F)CNC(*)=O] | (16) |
| 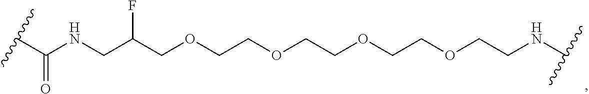 [SMILES: *NCCOCCOCCOCCOCC(F)CNC(*)=O] | (17) |
| 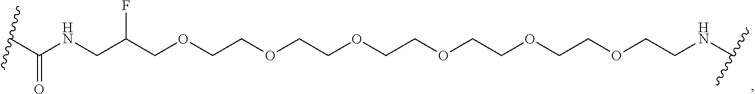 [SMILES: *NCCOCCOCCOCCOCCOCCOCC(F)CNC(*)=O] | (18) |
| 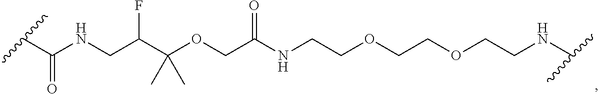 [SMILES: *NCCOCCOCCNC(=O)COC(C)(C)C(F)CNC(*)=O] | (19) |
| 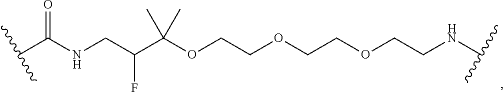 [SMILES: *NCCOCCOCCOC(C)(C)C(F)CNC(*)=O] | (20) |
| 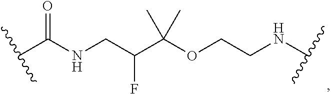 [SMILES: *NCCOC(C)(C)C(F)CNC(*)=O] | (21) |
| 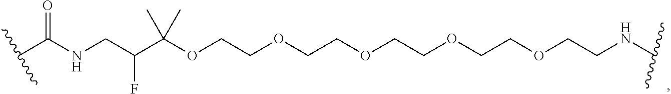 [SMILES: *NCCOCCOCCOCCOCCOC(C)(C)C(F)CNC(*)=O] | (22) |
| 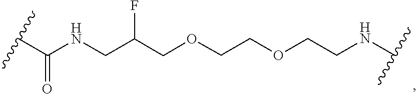 [SMILES: *NCCOCCOCC(F)CNC(*)=O] | (23) |
| 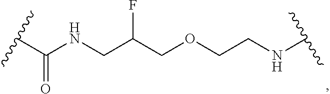 [SMILES: *NCCOCC(F)CNC(*)=O] | (24) |
| 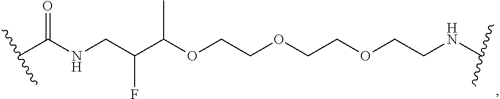 [SMILES: *NCCOCCOCCOC(C)C(F)CNC(*)=O] | (25) |
| 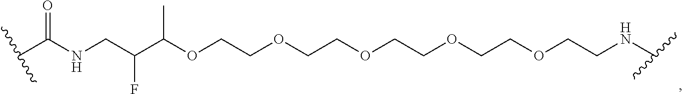 [SMILES: *NCCOCCOCCOCCOCCOC(C)C(F)CNC(*)=O] | (26) |
| 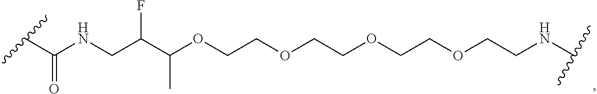 [SMILES: *NCCOCCOCCOCCOC(C)C(F)CNC(*)=O] | (27) |
| 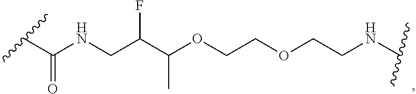 [SMILES: *NCCOCCOC(C)C(F)CNC(*)=O] | (28) |
| 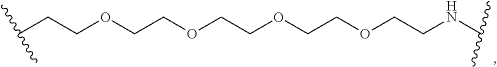 [SMILES: *CCOCCOCCOCCOCCN*] | (29) |
| 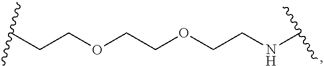 [SMILES: *CCOCCOCCN*] | (30) |
| 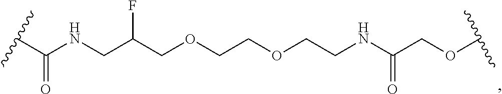 [SMILES: *OCC(=O)NCCOCCOCC(F)CNC(*)=O] | (31) |
| 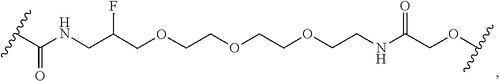 [SMILES: *OCC(=O)NCCOCCOCCOCC(F)CNC(*)=O] | (32) |
| 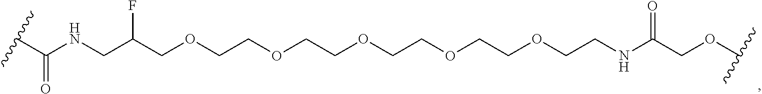 [SMILES: *OCC(=O)NCCOCCOCCOCCOCCOCC(F)CNC(*)=O] | (33) |
| 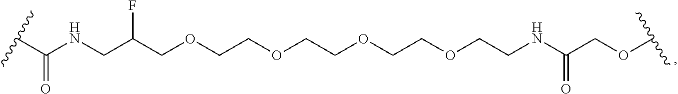 [SMILES: *OCC(=O)NCCOCCOCCOCCOCC(F)CNC(*)=O] | (34) |
| 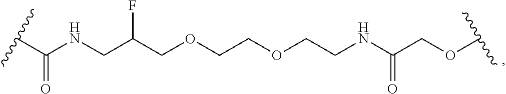 [SMILES: *OCC(=O)NCCOCCOCC(F)CNC(*)=O] | (35) |
| 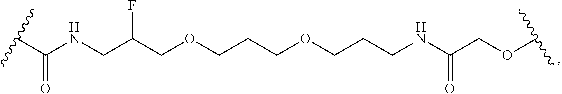 [SMILES: *OCC(=O)NCCCOCCCOCC(F)CNC(*)=O] | (36) |
| 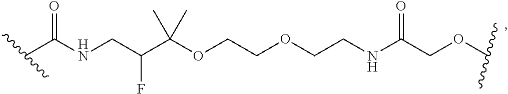 [SMILES: *OCC(=O)NCCOCCOC(C)(C)C(F)CNC(*)=O] | (37) |
| 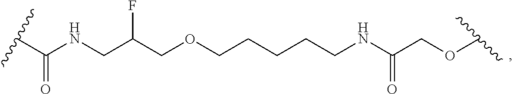 [SMILES: *OCC(=O)NCCCCCOCC(F)CNC(*)=O] | (38) |
| 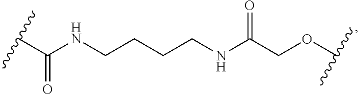 [SMILES: *OCC(=O)NCCCCNC(*)=O] | (39) |
| 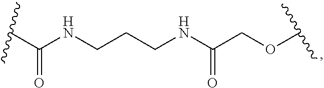 [SMILES: *OCC(=O)NCCCNC(*)=O] | (40) |
| 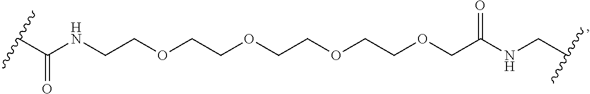 [SMILES: *CNC(=O)COCCOCCOCCOCCNC(*)=O] | (41) |
| 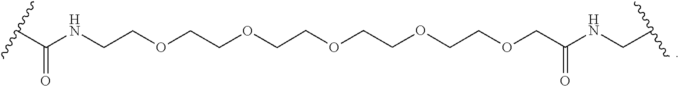 [SMILES: *CNC(=O)COCCOCCOCCOCCOCCNC(*)=O] | (42) |
| 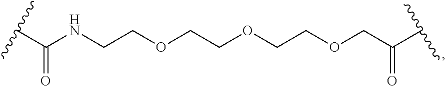 [SMILES: *C(=O)COCCOCCOCCNC(*)=O] | (43) |
| 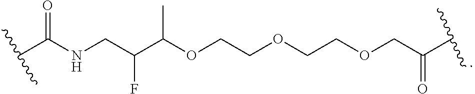 [SMILES: *C(=O)COCCOCCOC(C)C(F)CNC(*)=O] | (44) |
| 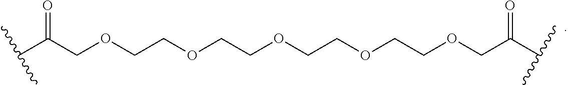 [SMILES: *C(=O)COCCOCCOCCOCCOCC(*)=O] | (45) |
| 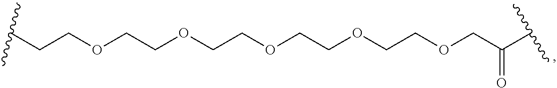 [SMILES: *CCOCCOCCOCCOCCOCC(*)=O] | (46) |
| 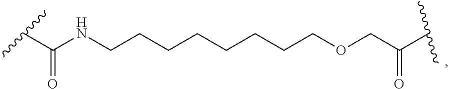 [SMILES: *C(=O)COCCCCCCCCNC(*)=O] | (47) |
| 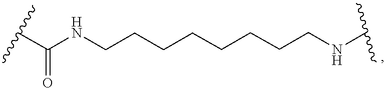 [SMILES: *NCCCCCCCCNC(*)=O] | (49) |
| 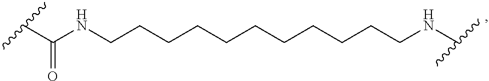 [SMILES: *NCCCCCCCCCCCNC(*)=O] | (50) |
| 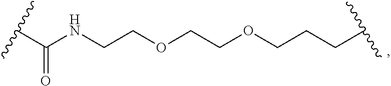 [SMILES: *CCCOCCOCCNC(*)=O] | (51) |
| 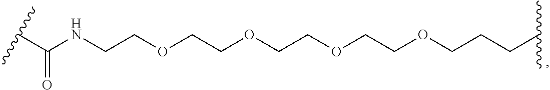 [SMILES: *CCCOCCOCCOCCOCCNC(*)=O] | (52) |
| 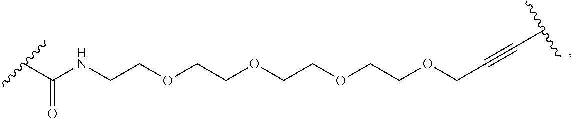 [SMILES: *C#CCOCCOCCOCCOCCNC(*)=O] | (53) |
| 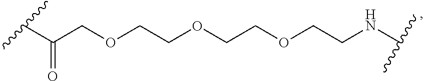 [SMILES: *NCCOCCOCCOCC(*)=O] | (54) |
| 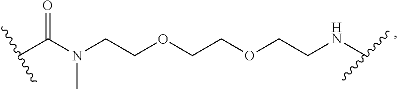 [SMILES: *NCCOCCOCCN(C)C(*)=O] | (55) |
| 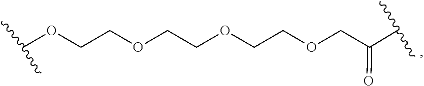 [SMILES: *OCCOCCOCCOCC(*)=O] | (56) |
| 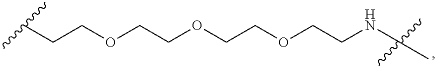 [SMILES: *CCOCCOCCOCCN*] | (57) |
| 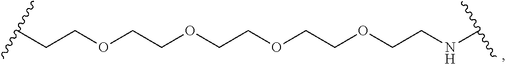 [SMILES: *CCOCCOCCOCCOCCN*] | (58) |
| 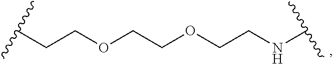 [SMILES: *CCOCCOCCN* ; ","] | (59) |
| 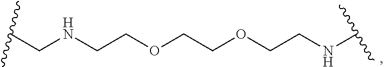 [SMILES: *CNCCOCCOCCN*] | (60) |
| 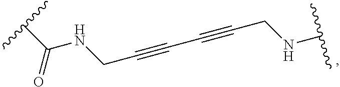 [SMILES: *NCC#CC#CCNC(*)=O] | (61) |
| 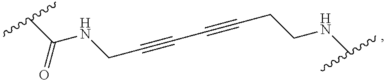 [SMILES: *NCCC#CC#CCNC(*)=O] | (62) |
| 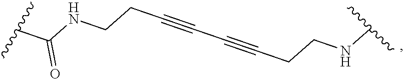 [SMILES: *NCCC#CC#CCCNC(*)=O] | (63) |
| 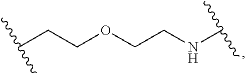 [SMILES: *CCOCCN*] | (64) |
| 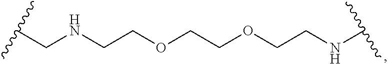 [SMILES: *CNCCOCCOCCN* ; ","] | (65) |
| 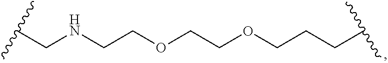 [SMILES: *CCCOCCOCCNC*] | (66) |
| 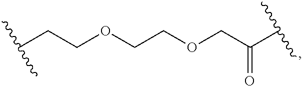 [SMILES: *CCOCCOCC(*)=O] | (67) |
| 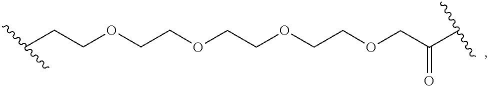 [SMILES: *CCOCCOCCOCCOCC(*)=O] | (68) |
| 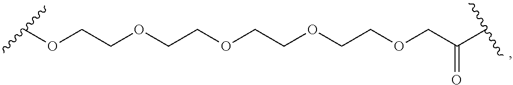 [SMILES: *OCCOCCOCCOCCOCC(*)=O ; ","] | (69) |
| 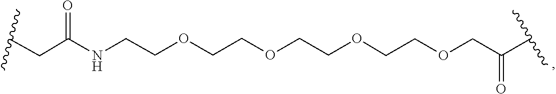 [SMILES: *CC(=O)NCCOCCOCCOCCOCC(*)=O ; ","] | (70) |
| 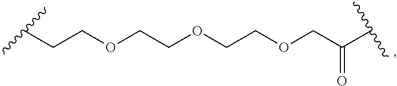 [SMILES: *CCOCCOCCOCC(*)=O] | (71) |
| 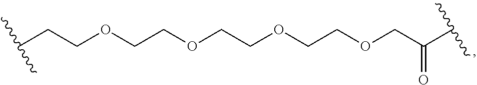 [SMILES: *CCOCCOCCOCCOCC(*)=O] | (72) |
| 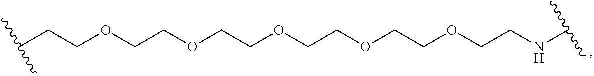 [SMILES: *CCOCCOCCOCCOCCOCCN*] | (73) |
| 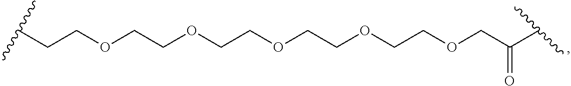 [SMILES: *CCOCCOCCOCCOCCOCC(*)=O] | (74) |
| 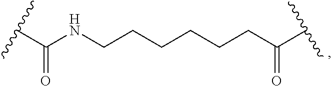 [SMILES: *C(=O)CCCCCCNC(*)=O] | (75) |
| 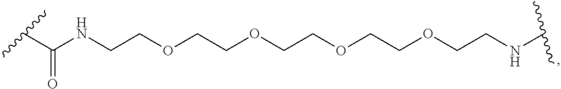 [SMILES: *NCCOCCOCCOCCOCCNC(*)=O] | (76) |
| 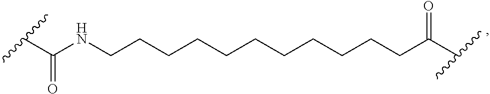 [SMILES: *C(=O)CCCCCCCCCCCNC(*)=O] | (77) |
| 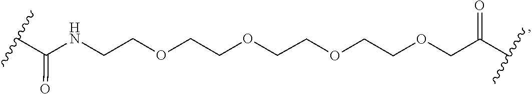 [SMILES: *C(=O)COCCOCCOCCOCCNC(*)=O] | (78) |
| 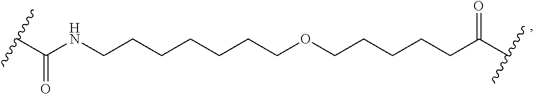 [SMILES: *C(=O)CCCCCOCCCCCCCNC(*)=O] | (79) |
| 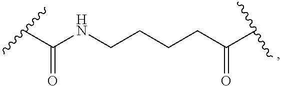 [SMILES: *C(=O)CCCCNC(*)=O] | (80) |
| 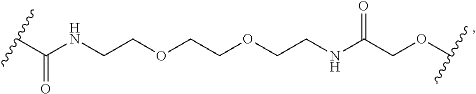 [SMILES: *OCC(=O)NCCOCCOCCNC(*)=O] | (81) |
| 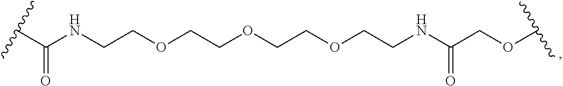 [SMILES: *OCC(=O)NCCOCCOCCOCCNC(*)=O] | (82) |
| 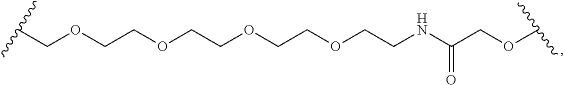 [SMILES: *COCCOCCOCCOCCNC(=O)CO*] | (83) |
| 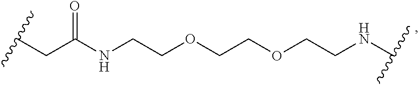 [SMILES: *CC(=O)NCCOCCOCCN*] | (84) |
| 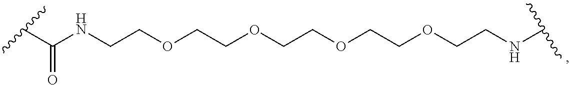 [SMILES: *NCCOCCOCCOCCOCCNC(*)=O] | (85) |
| 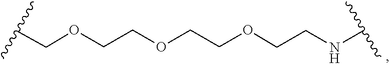 [SMILES: *COCCOCCOCCN*] | (86) |
| 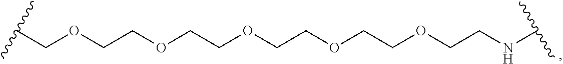 [SMILES: *COCCOCCOCCOCCOCCN*] | (87) |
| 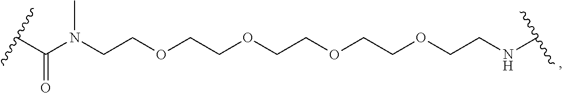 [SMILES: *NCCOCCOCCOCCOCCN(C)C(*)=O] | (88) |
| 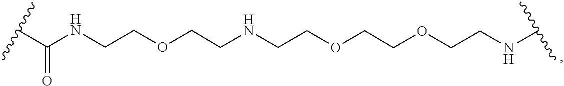 [SMILES: *NCCOCCOCCNCCOCCNC(*)=O] | (89) |
| 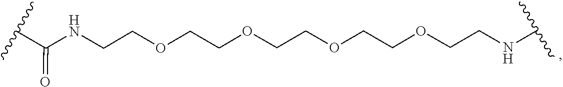 [SMILES: *NCCOCCOCCOCCOCCNC(*)=O] | (90) |
| 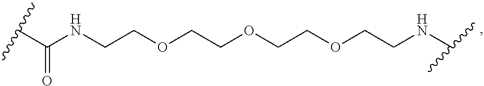 [SMILES: *NCCOCCOCCOCCNC(*)=O] | (91) |
| 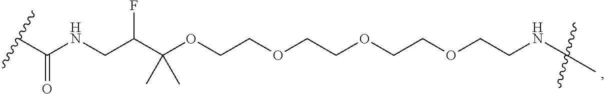 [SMILES: *NCCOCCOCCOCCOC(C)(C)C(F)CNC(*)=O] | (92) |
| 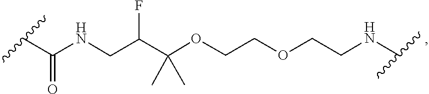 [SMILES: *NCCOCCOC(C)(C)C(F)CNC(*)=O] | (93) |
| 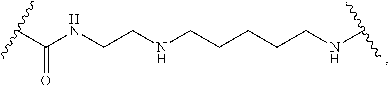 [SMILES: *NCCCCCNCCNC(*)=O] | (94) |
| 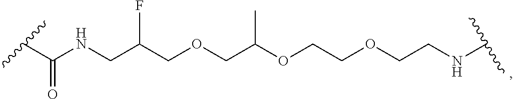 [SMILES: *NCCOCCOC(C)COCC(F)CNC(*)=O] | (95) |
| 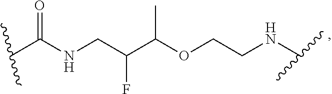 [SMILES: *NCCOC(C)C(F)CNC(*)=O] | (96) |
| 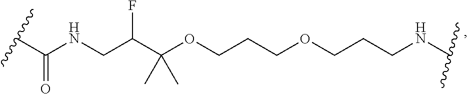 [SMILES: *NCCCOCCCOC(C)(C)C(F)CNC(*)=O] | (97) |
| 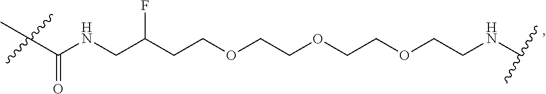 [SMILES: *NCCOCCOCCOCCC(F)CNC(*)=O] | (98) |
| 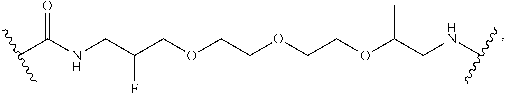 [SMILES: *NCC(C)OCCOCCOCC(F)CNC(*)=O] | (99) |
| 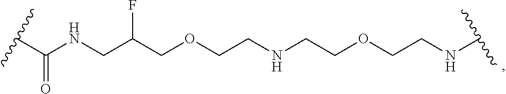 [SMILES: *NCCOCCNCCOCC(F)CNC(*)=O] | (100) |
| 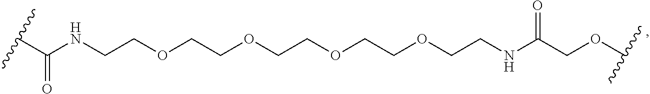 [SMILES: *OCC(=O)NCCOCCOCCOCCOCCNC(*)=O] | (101) |
| 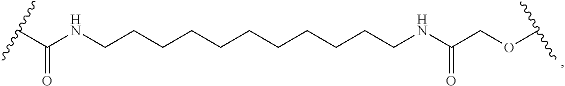 [SMILES: *OCC(=O)NCCCCCCCCCCCNC(*)=O] | (102) |
| 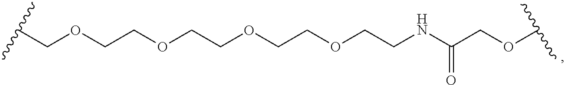 [SMILES: *COCCOCCOCCOCCNC(=O)CO*] | (103) |
| 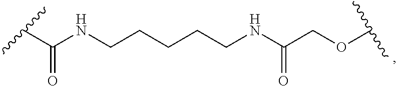 [SMILES: *OCC(=O)NCCCCCNC(*)=O] | (104) |
| 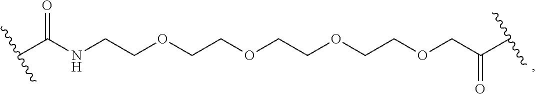 [SMILES: *C(=O)COCCOCCOCCOCCNC(*)=O] | (105) |
| 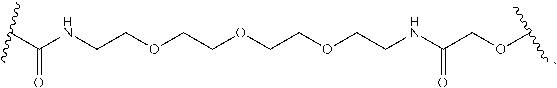 [SMILES: *OCC(=O)NCCOCCOCCOCCNC(*)=O] | (106) |
| 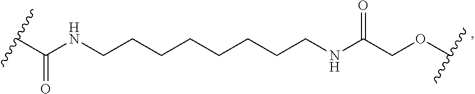 [SMILES: *OCC(=O)NCCCCCCCCNC(*)=O] | (107) |
| 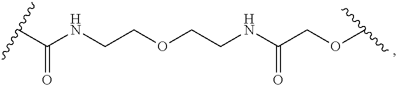 [SMILES: *OCC(=O)NCCOCCNC(*)=O] | (108) |
| 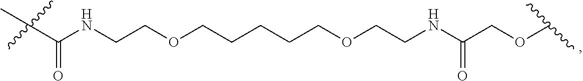 [SMILES: *OCC(=O)NCCOCCCCCOCCNC(*)=O] | (109) |
| 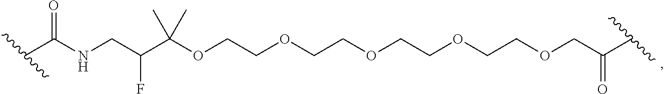 [SMILES: *C(=O)COCCOCCOCCOCCOC(C)(C)C(F)CNC(*)=O] | (110) |
| 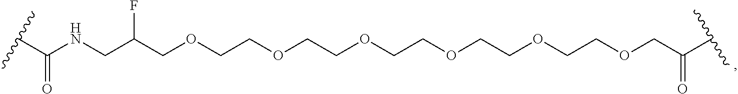 [SMILES: *C(=O)COCCOCCOCCOCCOCCOCC(F)CNC(*)=O] | (111) |
| 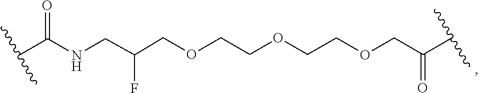 [SMILES: *C(=O)COCCOCCOCC(F)CNC(*)=O] | (112) |
| 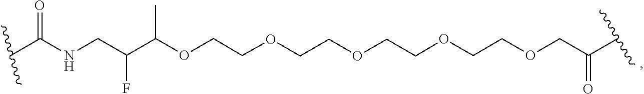 [SMILES: *C(=O)COCCOCCOCCOCCOC(C)C(F)CNC(*)=O] | (113) |
| 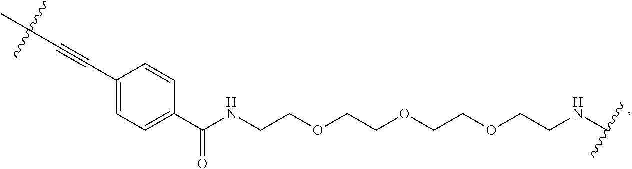 [SMILES: *C#Cc1ccc(C(=O)NCCOCCOCCOCCN*)cc1] | (114) |
| 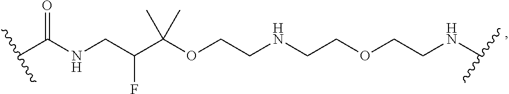 [SMILES: *NCCOCCNCCOC(C)(C)C(F)CNC(*)=O] | (115) |
| 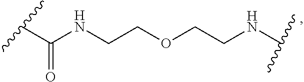 [SMILES: *NCCOCCNC(*)=O] | (116) |
| 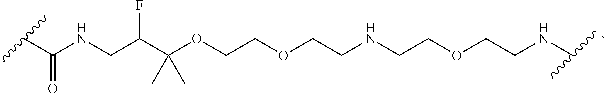 [SMILES: *NCCOCCNCCOCCOC(C)(C)C(F)CNC(*)=O] | (117) |
| 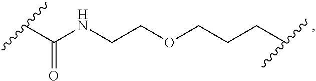 [SMILES: *CCCOCCNC(*)=O] | (118) |
| 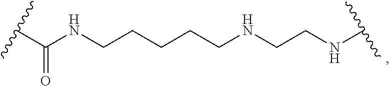 [SMILES: *NCCNCCCCCNC(*)=O] | |
| 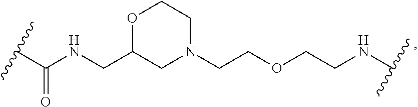 [SMILES: *NCCOCCN1CCOC(CNC(*)=O)C1] | (119) |
| 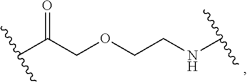 [SMILES: *NCCOCC(*)=O] | |
| 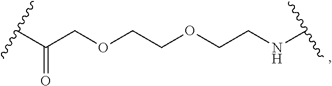 [SMILES: *NCCOCCOCC(*)=O] | (120) |
| 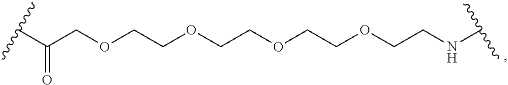 [SMILES: *NCCOCCOCCOCCOCC(*)=O] | (121) |
| 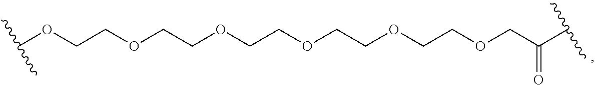 [SMILES: *OCCOCCOCCOCCOCCOCC(*)=O] | (122) |
| 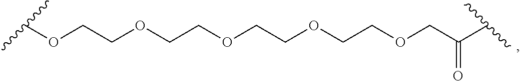 [SMILES: *OCCOCCOCCOCCOCC(*)=O] | (123) |
| 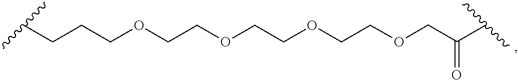 [SMILES: *CCCOCCOCCOCCOCC(*)=O] | (124) |
| 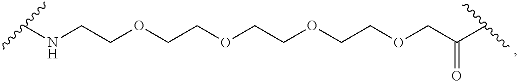 [SMILES: *NCCOCCOCCOCCOCC(*)=O] | (125) |
| 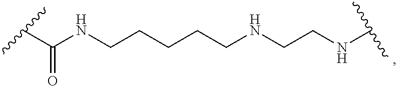 [SMILES: *NCCNCCCCCNC(*)=O] | (126) |
| 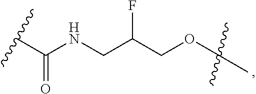 [SMILES: *OCC(F)CNC(*)=O] | |
| 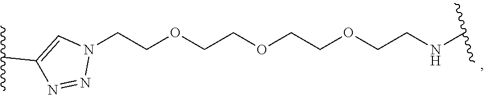 [SMILES: *NCCOCCOCCOCCn1cc(*)nn1] | (127) |
| 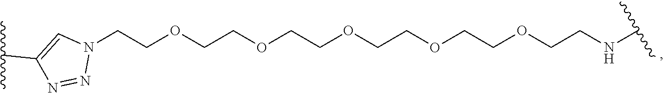 [SMILES: *NCCOCCOCCOCCOCCOCCn1cc(*)nn1] | (128) |
| 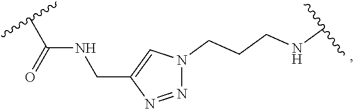 [SMILES: *NCCCn1cc(CNC(*)=O)nn1] | (129) |
| 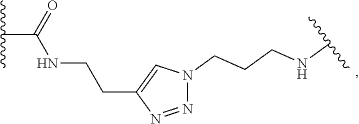 [SMILES: *NCCCn1cc(CCNC(*)=O)nn1] | (130) |
| 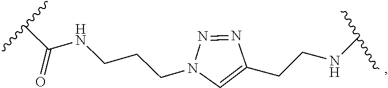 [SMILES: *NCCc1cn(CCCNC(*)=O)nn1] | (131) |
| 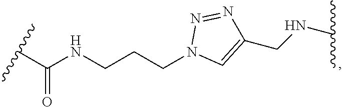 [SMILES: *NCc1cn(CCCNC(*)=O)nn1 ; ","] | (132) |
| 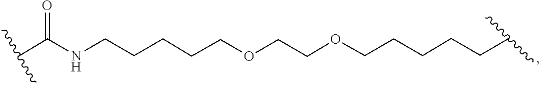 [SMILES: *CCCCCOCCOCCCCCNC(*)=O ; ","] | (133) |
| 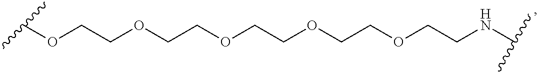 [SMILES: "," ; *NCCOCCOCCOCCOCCO*] | (134) |
| 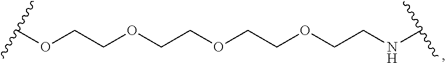 [SMILES: *NCCOCCOCCOCCO* ; ","] | (135) |
| 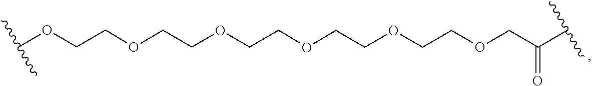 [SMILES: *OCCOCCOCCOCCOCCOCC(*)=O ; ","] | (136) |
| 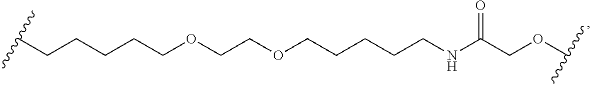 [SMILES: *CCCCCOCCOCCCCCNC(=O)CO* ; ","] | (137) |
| 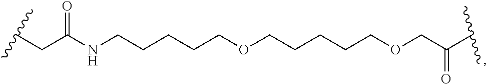 [SMILES: *CC(=O)NCCCCCOCCCCCOCC(*)=O ; ","] | (138) |
| 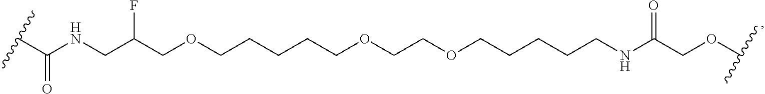 [SMILES: *OCC(=O)NCCCCCOCCOCCCCCOCC(F)CNC(*)=O ; ","] | (139) |
| 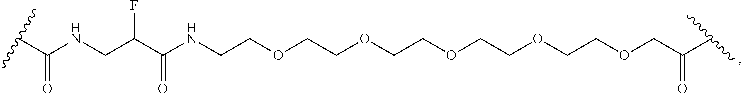 [SMILES: *C(=O)COCCOCCOCCOCCOCCNC(=O)C(F)CNC(*)=O ; ","] | (140) |
| 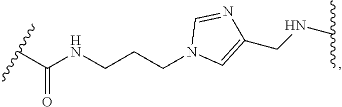 [SMILES: *NCc1cn(CCCNC(*)=O)cn1 ; ","] | (141) |
| 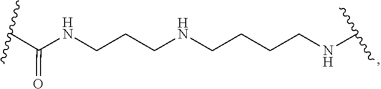 [SMILES: *NCCCCNCCCNC(*)=O ; ","] | (142) |
| 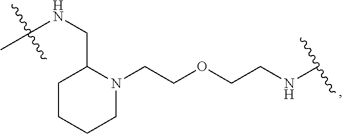 [SMILES: *NCCOCCN1CCCCC1CN* ; ","] | (143) |
| 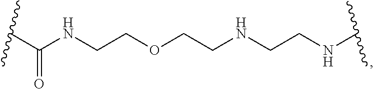 [SMILES: *NCCNCCOCCNC(*)=O ; ","] | (144) |
| 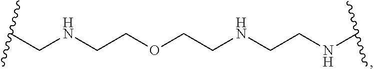 [SMILES: *CNCCOCCNCCN* ; ","] | (145) |
| 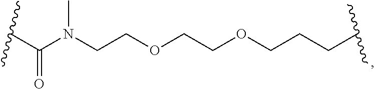 [SMILES: *CCCOCCOCCN(C)C(*)=O ; ","] | (146) |
| 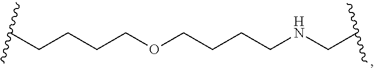 [SMILES: *CCCCOCCCCNC* ; ","] | (147) |
| 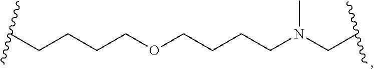 [SMILES: *CCCCOCCCCN(C)C* ; ","] | (148) |
| 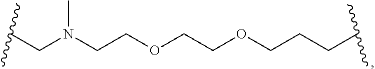 [SMILES: *CCCOCCOCCN(C)C* ; ","] | (149) |
| 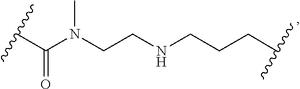 [SMILES: *CCCNCCN(C)C(*)=O ; ","] | (150) |
| 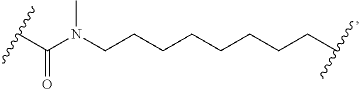 [SMILES: *CCCCCCCCN(C)C(*)=O ; ","] | (151) |
| 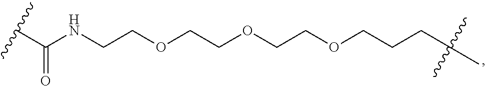 [SMILES: *CCCOCCOCCOCCNC(*)=O ; ","] | (152) |
| 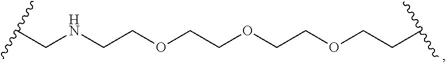 [SMILES: *CCOCCOCCOCCNC* ; ","] | (153) |
| 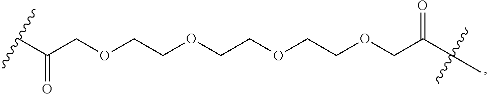 [SMILES: *C(=O)COCCOCCOCCOCC(*)=O ; ","] | (154) |
| 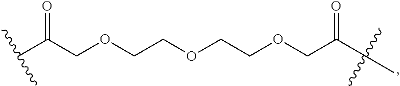 [SMILES: *C(=O)COCCOCCOCC(*)=O ; ","] | (155) |
| 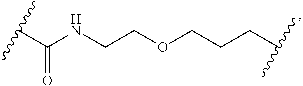 [SMILES: "," ; *CCCOCCNC(*)=O] | (156) |
| 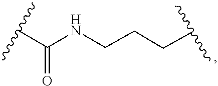 [SMILES: *CCCNC(*)=O ; ","] | (157) |
| 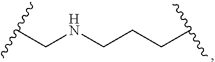 [SMILES: *CCCNC* ; ","] | (158) |
| 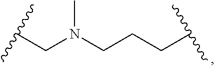 [SMILES: *CCCN(C)C* ; ","] | (159) |
| 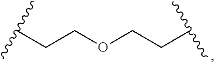 [SMILES: *CCOCC* ; ","] | (160) |
| 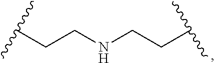 [SMILES: *CCNCC* ; ","] | (161) |
| 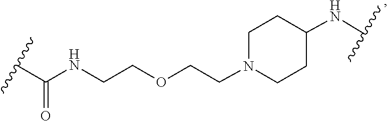 [SMILES: "," ; *NC1CCN(CCOCCNC(*)=O)CC1] | (162) |
| 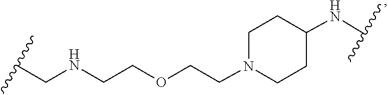 [SMILES: "," ; *CNCCOCCN1CCC(N*)CC1] | (163) |
| 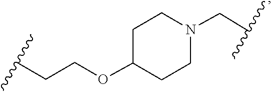 [SMILES: "," ; *CCOC1CCN(C*)CC1] | (164) |
| 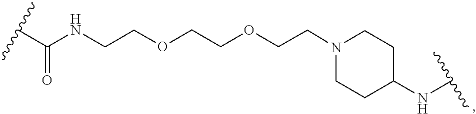 [SMILES: *NC1CCN(CCOCCOCCNC(*)=O)CC1 ; ","] | (165) |
| 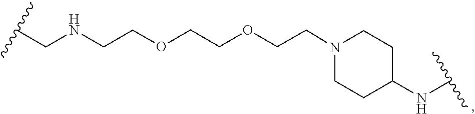 [SMILES: *CNCCOCCOCCN1CCC(N*)CC1 ; ","] | (166) |
| 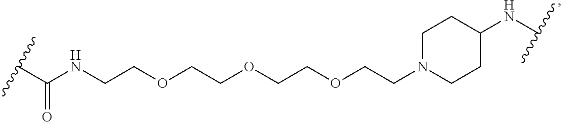 [SMILES: "," ; *NC1CCN(CCOCCOCCOCCNC(*)=O)CC1] | (167) |
| 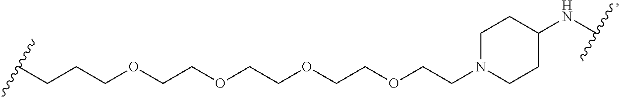 [SMILES: "," ; *CCCOCCOCCOCCOCCN1CCC(N*)CC1] | (168) |
| 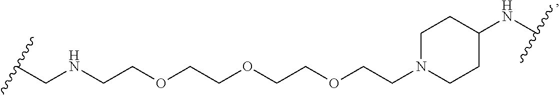 [SMILES: "," ; *CNCCOCCOCCOCCN1CCC(N*)CC1] | (169) |
| 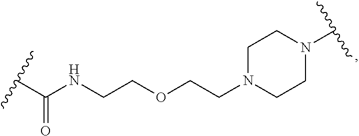 [SMILES: *C(=O)NCCOCCN1CCN(*)CC1 ; ","] | (170) |
| 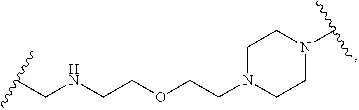 [SMILES: *CNCCOCCN1CCN(*)CC1 ; ","] | (171) |
| 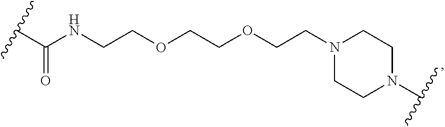 [SMILES: *C(=O)NCCOCCOCCN1CCN(*)CC1 ; ","] | (172) |
| 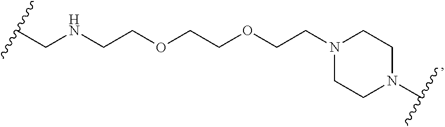 [SMILES: *CNCCOCCOCCN1CCN(*)CC1 ; ","] | (173) |
| 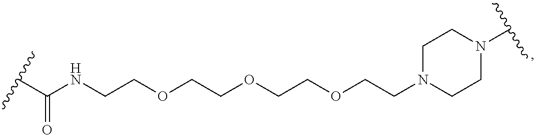 [SMILES: *C(=O)NCCOCCOCCOCCN1CCN(*)CC1 ; ","] | (174) |
| 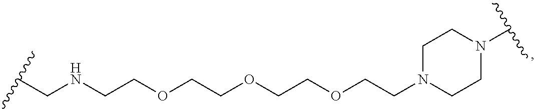 [SMILES: *CNCCOCCOCCOCCN1CCN(*)CC1 ; ","] | (175) |
| 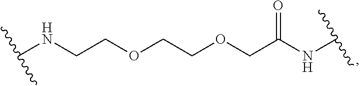 [SMILES: *NCCOCCOCC(=O)N*] | (176) |
| 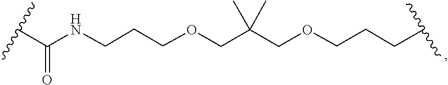 [SMILES: *CCCOCC(C)(C)COCCCNC(*)=O ; ","] | (177) |
| 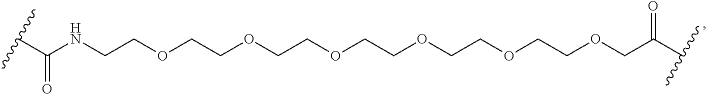 [SMILES: *C(=O)COCCOCCOCCOCCOCCOCCNC(*)=O ; ","] | (178) |
| 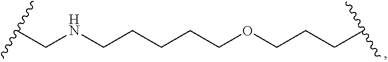 [SMILES: *CCCOCCCCCNC*] | (179) |
| 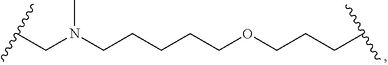 [SMILES: *CCCOCCCCCN(C)C*] | (180) |
| 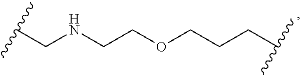 [SMILES: "," ; *CCCOCCNC*] | (181) |
| 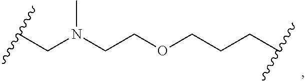 [SMILES: *CCCOCCN(C)C*] | (182) |
| 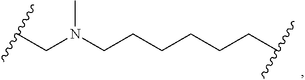 [SMILES: *CCCCCCN(C)C*] | (183) |
| 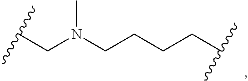 [SMILES: *CCCCN(C)C*] | (184) |
| 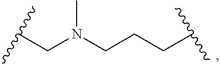 [SMILES: *CCCN(C)C*] | (185) |
| 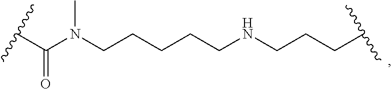 [SMILES: *CCCNCCCCCN(C)C(*)=O] | (186) |
| 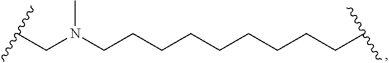 [SMILES: *CCCCCCCCCN(C)C*] | (187) |
| 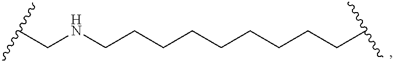 [SMILES: *CCCCCCCCCNC*] | (188) |
| 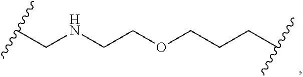 [SMILES: *CCCOCCNC*] | (189) |
| 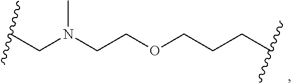 [SMILES: *CCCOCCN(C)C*] | (190) |
| 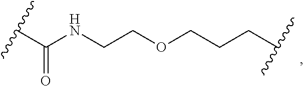 [SMILES: *CCCOCCNC(*)=O] | (191) |
| 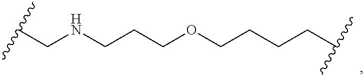 [SMILES: *CCCCOCCCNC*] | (192) |
| 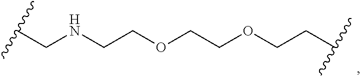 [SMILES: *CCOCCOCCNC*] | (193) |
| 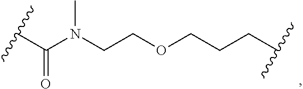 [SMILES: *CCCOCCN(C)C(*)=O] | (194) |
| 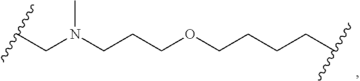 [SMILES: *CCCCOCCCN(C)C*] | (195) |
| 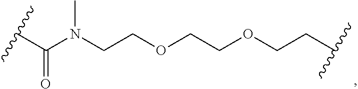 [SMILES: *CCOCCOCCN(C)C(*)=O] | (196) |
| 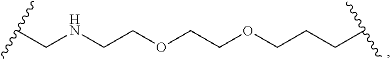 [SMILES: *CCCOCCOCCNC*] | (197) |
| 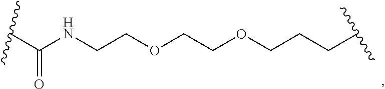 [SMILES: *CCCOCCOCCNC(*)=O] | (198) |
| 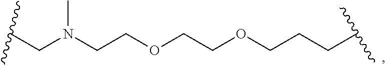 [SMILES: *CCCOCCOCCN(C)C*] | (199) |
| 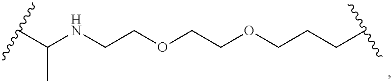 [SMILES: *CCCOCCOCCNC(*)C] | (200) |
| 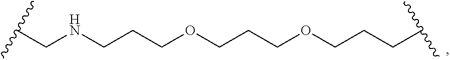 [SMILES: *CCCOCCCOCCCNC*] | (201) |
| 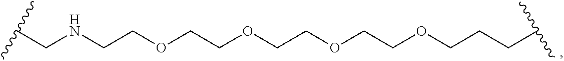 [SMILES: *CCCOCCOCCOCCOCCNC*] | (202) |
| 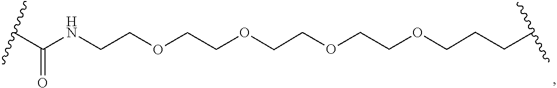 [SMILES: *CCCOCCOCCOCCOCCNC(*)=O] | (203) |
| 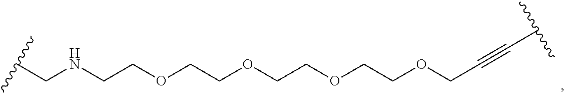 [SMILES: *C#CCOCCOCCOCCOCCNC*] | (204) |
| 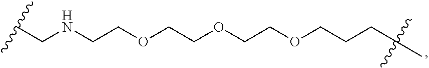 [SMILES: *CCCOCCOCCOCCNC*] | (205) |
| 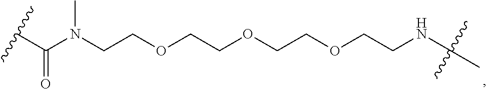 [SMILES: *NCCOCCOCCOCCN(C)C(*)=O] | (206) |
| 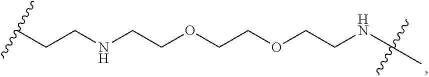 [SMILES: *CCNCCOCCOCCN*] | (207) |
| 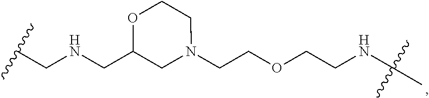 [SMILES: *CNCC1CN(CCOCCN*)CCO1] | (208) |
| 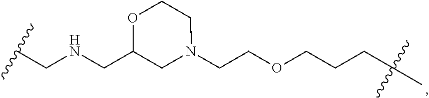 [SMILES: *CCCOCCN1CCOC(CNC*)C1] | (209) |
| 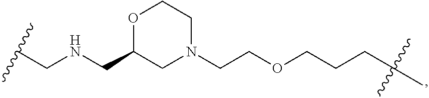 [SMILES: *CCCOCCN1CCO[C@H](CNC*)C1] | (210) |
| 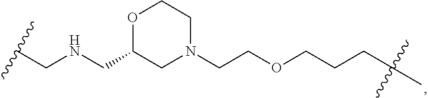 [SMILES: *CCCOCCN1CCO[C@@H](CNC*)C1] | (211) |
| 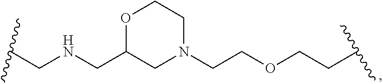 [SMILES: *CCOCCN1CCOC(CNC*)C1] | (212) |
| 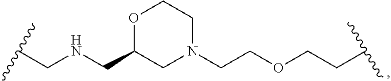 [SMILES: *CCOCCN1CCO[C@H](CNC*)C1] | (213) |
| 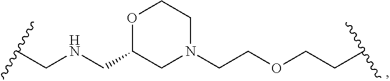 [SMILES: *CCOCCN1CCO[C@@H](CNC*)C1] | (214) |
| 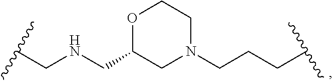 [SMILES: *CCCN1CCO[C@@H](CNC*)C1] | (215) |
| 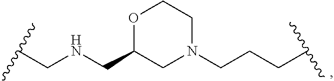 [SMILES: *CCCN1CCO[C@H](CNC*)C1] | (216) |
| 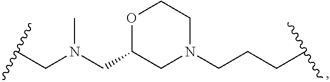 [SMILES: *CCCN1CCO[C@@H](CN(C)C*)C1] | (217) |
| 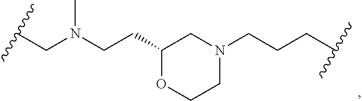 [SMILES: *CCCN1CCO[C@H](CCN(C)C*)C1] | (218) |
| 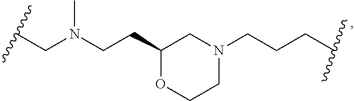 [SMILES: *CCCN1CCO[C@@H](CCN(C)C*)C1] | (219) |
| 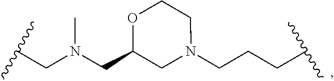 [SMILES: *CCCN1CCO[C@H](CN(C)C*)C1] | (220) |
| 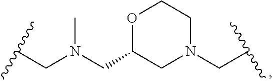 [SMILES: *CN(C)C[C@H]1CN(C*)CCO1] | (221) |
| 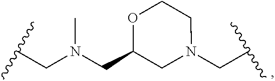 [SMILES: *CN(C)C[C@@H]1CN(C*)CCO1] | (222) |
| 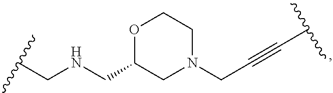 [SMILES: *C#CCN1CCO[C@@H](CNC*)C1] | (223) |
| 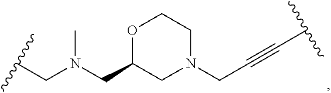 [SMILES: *C#CCN1CCO[C@H](CN(C)C*)C1] | (224) |
| 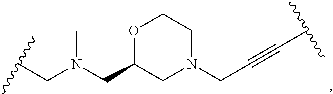 [SMILES: *C#CCN1CCO[C@H](CN(C)C*)C1] | (225) |
| 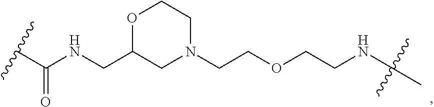 [SMILES: *NCCOCCN1CCOC(CNC(*)=O)C1] | (226) |
| 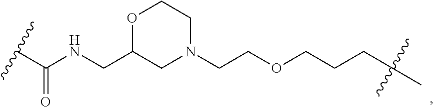 [SMILES: *CCCOCCN1CCOC(CNC(*)=O)C1] | (227) |
| 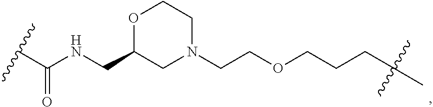 [SMILES: *CCCOCCN1CCO[C@H](CNC(*)=O)C1] | (228) |
| 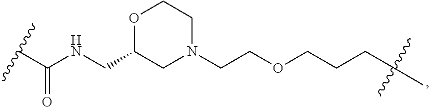 [SMILES: *CCCOCCN1CCO[C@@H](CNC(*)=O)C1] | (229) |
| 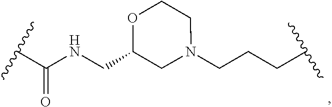 [SMILES: *CCCN1CCO[C@@H](CNC(*)=O)C1] | (230) |
| 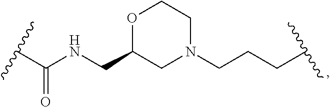 [SMILES: *CCCN1CCO[C@H](CNC(*)=O)C1] | (231) |
| 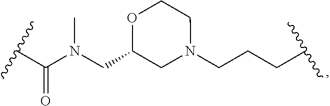 [SMILES: *CCCN1CCO[C@@H](CN(C)C(*)=O)C1] | (232) |
| 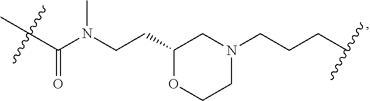 [SMILES: *CCCN1CCO[C@H](CCN(C)C(*)=O)C1] | (233) |
| 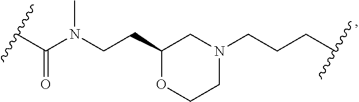 [SMILES: *CCCN1CCO[C@@H](CCN(C)C(*)=O)C1] | (234) |
| 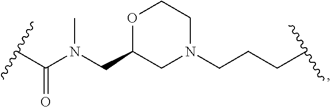 [SMILES: *CCCN1CCO[C@H](CN(C)C(*)=O)C1] | (235) |
| 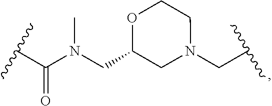 [SMILES: *CN1CCO[C@@H](CN(C)C(*)=O)C1] | (236) |
| 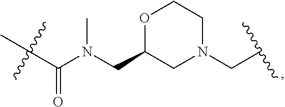 [SMILES: *CN1CCO[C@H](CN(C)C(*)=O)C1] | (237) |
| 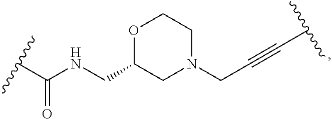 [SMILES: *C#CCN1CCO[C@@H](CNC(*)=O)C1] | (238) |
| 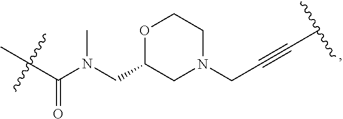 [SMILES: *C#CCN1CCO[C@@H](CN(C)C(*)=O)C1] | (239) |
| 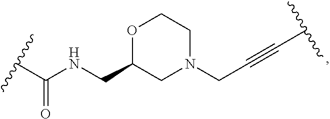 [SMILES: *C#CCN1CCO[C@H](CNC(*)=O)C1] | (240) |
| 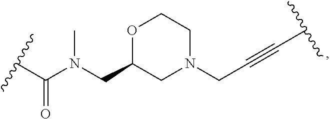 [SMILES: *C#CCN1CCO[C@H](CN(C)C(*)=O)C1] | (241) |
| 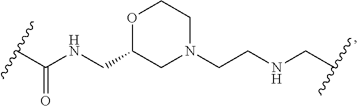 [SMILES: *CNCCN1CCO[C@@H](CNC(*)=O)C1] | (242) |
| 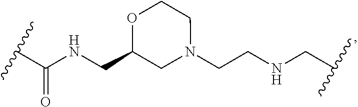 [SMILES: *CNCCN1CCO[C@H](CNC(*)=O)C1] | (243) |
| 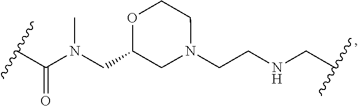 [SMILES: *CNCCN1CCO[C@@H](CN(C)C(*)=O)C1] | (244) |
| 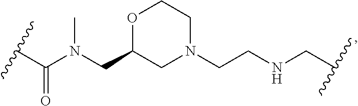 [SMILES: *CNCCN1CCO[C@H](CN(C)C(*)=O)C1] | (245) |
| 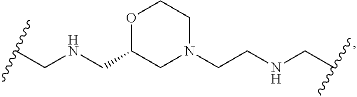 [SMILES: *CNCCN1CCO[C@@H](CNC*)C1] | (246) |
| 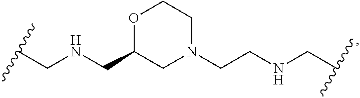 [SMILES: *CNCCN1CCO[C@H](CNC*)C1] | (247) |
| 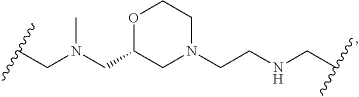 [SMILES: *CNCCN1CCO[C@@H](CN(C)C*)C1] | (248) |
| 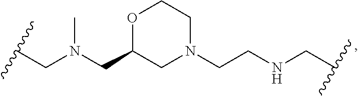 [SMILES: *CNCCN1CCO[C@H](CN(C)C*)C1] | (249) |
| 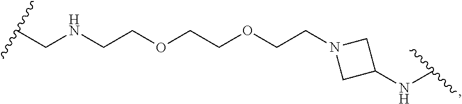 [SMILES: *CNCCOCCOCCN1CC(N*)C1] | (250) |
| 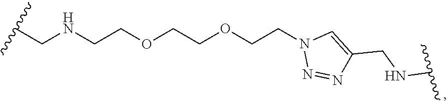 [SMILES: *CNCCOCCOCCn1cc(CN*)nn1] | (251) |
| 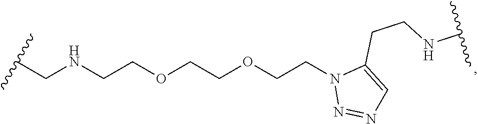 [SMILES: *CNCCOCCOCCn1nncc1CCN*] | (253) |
| 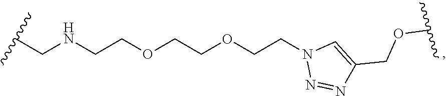 [SMILES: *CNCCOCCOCCn1cc(CO*)nn1] | (254) |
| 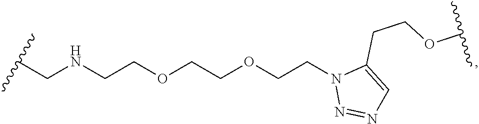 [SMILES: *CNCCOCCOCCn1nncc1CCO*] | (255) |
| 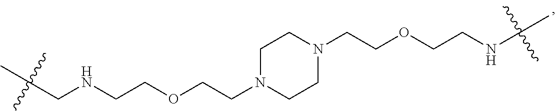 [SMILES: *CNCCOCCN1CCN(CCOCCN*)CC1] | (256) |
| 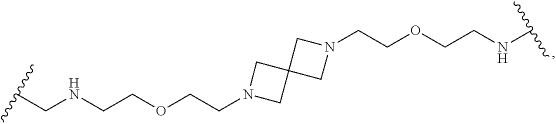 [SMILES: *CNCCOCCN1CC2(CN(CCOCCN*)C2)C1] | (257) |
| 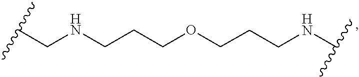 [SMILES: *CNCCCOCCCN*] | (258) |
| 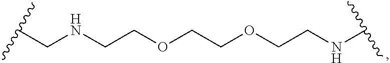 [SMILES: *CNCCOCCOCCN*] | (259) |
| 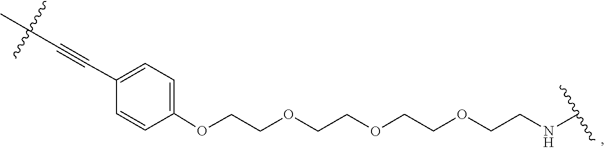 [SMILES: *C#Cc1ccc(OCCOCCOCCOCCN*)cc1] | (260) |
| 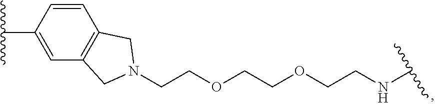 [SMILES: *NCCOCCOCCN1Cc2ccc(*)cc2C1] | (261) |
| 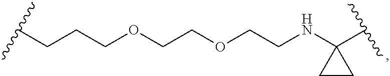 [SMILES: *CCCOCCOCCNC1(*)CC1] | (262) |
| 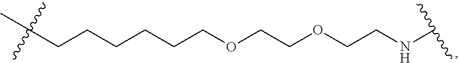 [SMILES: *CCCCCCOCCOCCN*] | (263) |
| 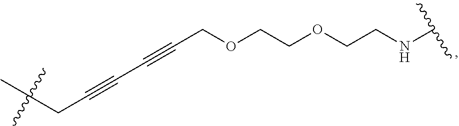 [SMILES: *CC#CC#CCOCCOCCN*] | (264) |
| 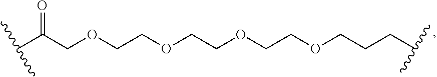 [SMILES: *CCCOCCOCCOCCOCC(*)=O] | (265) |
| 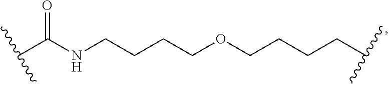 [SMILES: *CCCCOCCCCNC(*)=O] | (266) |
| 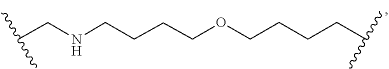 [SMILES: *CCCCOCCCCNC*] | (267) |
| 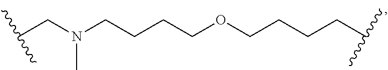 [SMILES: *CCCCOCCCCN(C)C*] | (268) |
| 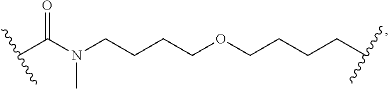 [SMILES: *CCCCOCCCCN(C)C(*)=O] | (269) |
| 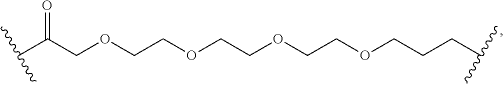 [SMILES: *CCCOCCOCCOCCOCC(*)=O] | (270) |
| 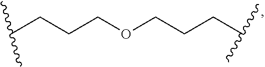 [SMILES: *CCCOCCC*] | (271) |
| 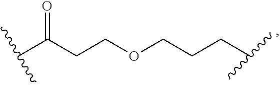 [SMILES: *CCCOCCC(*)=O] | (272) |
| 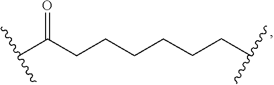 [SMILES: *CCCCCCC(*)=O] | (273) |
| 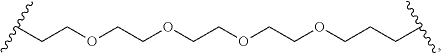 [SMILES: *CCCOCCOCCOCCOCC*] | (274) |
| 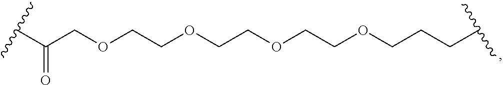 [SMILES: *CCCOCCOCCOCCOCC(*)=O] | (275) |
| 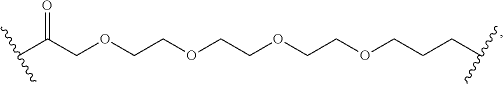 [SMILES: *CCCOCCOCCOCCOCC(*)=O] | (276) |
| 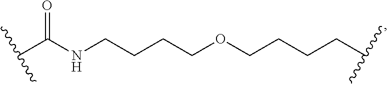 [SMILES: *CCCCOCCCCNC(*)=O] | (277) |
| 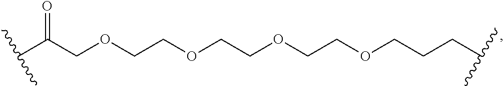 [SMILES: *CCCOCCOCCOCCOCC(*)=O] | (278) |
| 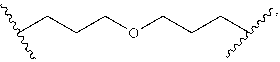 [SMILES: *CCCOCCC*] | (279) |
| 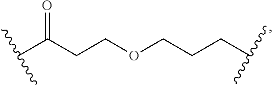 [SMILES: *CCCOCCC(*)=O] | (280) |
| 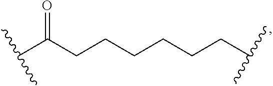 [SMILES: *CCCCCCC(*)=O] | (281) |
| 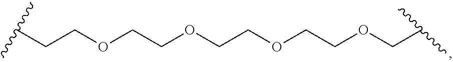 [SMILES: *CCOCCOCCOCCOC*] | (282) |
| 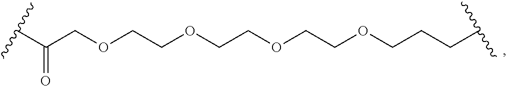 [SMILES: *CCCOCCOCCOCCOCC(*)=O] | (283) |
| 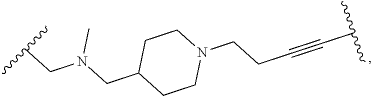 [SMILES: *C#CCCN1CCC(CN(C)C*)CC1] | (284) |
| 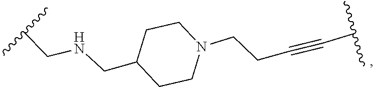 [SMILES: *C#CCCN1CCC(CNC*)CC1] | (285) |
| 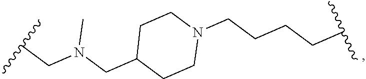 [SMILES: *CCCCN1CCC(CN(C)C*)CC1] | (286) |
| 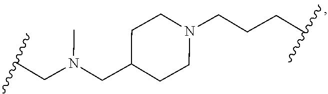 [SMILES: *CCCN1CCC(CN(C)C*)CC1] | (287) |
| 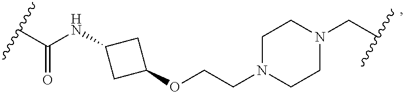 [SMILES: *CN1CCN(CCO[C@H]2C[C@H](NC(*)=O)C2)CC1] | (288) |
| 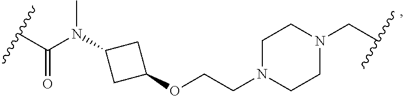 [SMILES: *CN1CCN(CCO[C@H]2C[C@H](N(C)C(*)=O)C2)CC1] | (289) |
| 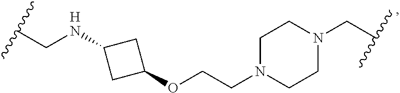 | (290) |
| 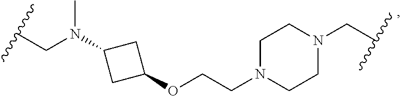 [SMILES: *CN1CCN(CCO[C@H]2C[C@H](N(C)C*)C2)CC1] | (291) |
| 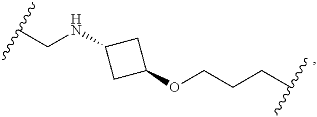 [SMILES: *CCCO[C@H]1C[C@H](NC*)C1] | (292) |
| 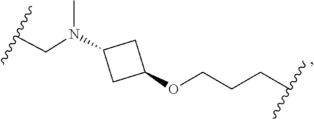 [SMILES: *CCCO[C@H]1C[C@H](N(C)C*)C1] | (293) |
| 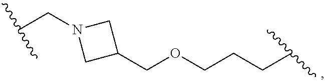 [SMILES: *CCCOCC1CN(C*)C1] | (294) |
| 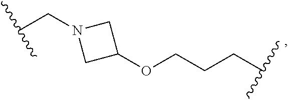 [SMILES: *CCCOC1CN(C*)C1] | (295) |
| 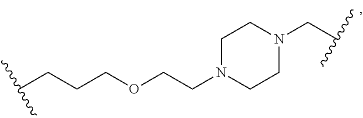 [SMILES: *CCCOCCN1CCN(C*)CC1] | (296) |
| 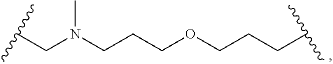 [SMILES: *CCCOCCCN(C)C*] | (297) |
| 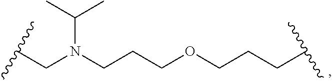 [SMILES: *CCCOCCCN(C*)C(C)C] | (298) |
| 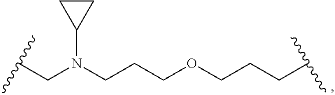 [SMILES: *CCCOCCCN(C*)C1CC1] | (299) |
| 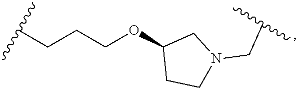 [SMILES: *CCCO[C@@H]1CCN(C*)C1] | (300) |
| 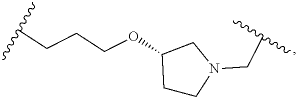 [SMILES: *CCCO[C@H]1CCN(C*)C1] | (301) |
| 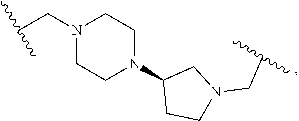 [SMILES: *CN1CCN([C@@H]2CCN(C*)C2)CC1] | (302) |
| 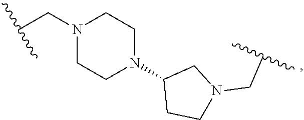 [SMILES: *CN1CCN([C@H]2CCN(C*)C2)CC1] | (303) |
| 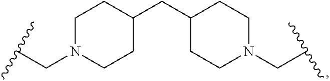 [SMILES: *CN1CCC(CC2CCN(C*)CC2)CC1] | (304) |
| 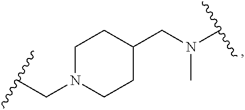 [SMILES: *CN1CCC(CN(*)C)CC1] | (305) |
| 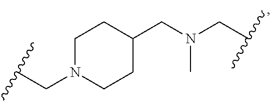 [SMILES: *CN(C)CC1CCN(C*)CC1] | (306) |
| 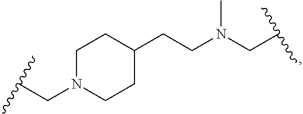 [SMILES: *CN(C)CCC1CCN(C*)CC1 ; ","] | (307) |
| 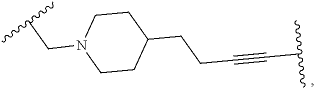 [SMILES: *C#CCCC1CCN(C*)CC1] | (308) |
| 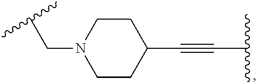 [SMILES: *C#CC1CCN(C*)CC1] | (309) |
| 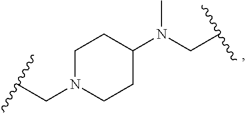 [SMILES: *CN1CCC(N(C)C*)CC1] | (310) |
| 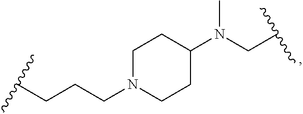 [SMILES: *CCCN1CCC(N(C)C*)CC1] | (311) |
| 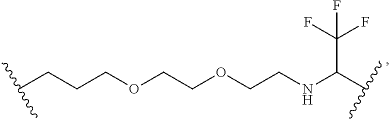 [SMILES: *CCCOCCOCCNC(*)C(F)(F)F] | (312) |
| 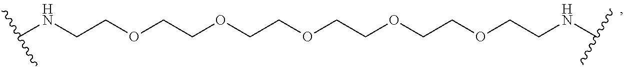 [SMILES: *NCCOCCOCCOCCOCCOCCN*] | (313) |
| 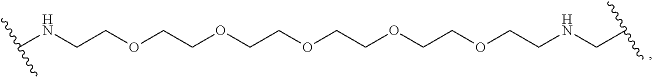 [SMILES: *CNCCOCCOCCOCCOCCOCCN*] | (314) |
| 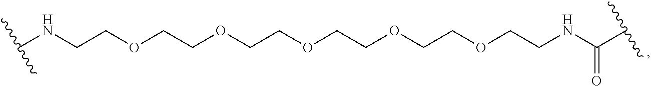 [SMILES: *NCCOCCOCCOCCOCCOCCNC(*)=O] | (315) |
| 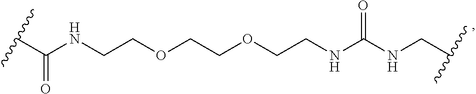 [SMILES: *CNC(=O)NCCOCCOCCNC(*)=O] | (316) |
| 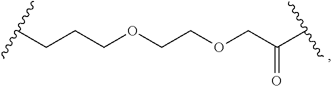 [SMILES: *CCCOCCOCC(*)=O] | (317) |
| 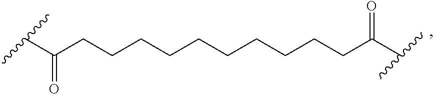 [SMILES: *C(=O)CCCCCCCCCCC(*)=O] | (318) |
| 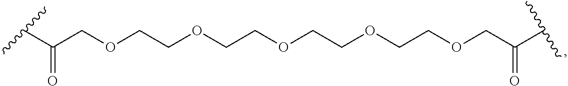 [SMILES: *C(=O)COCCOCCOCCOCCOCC(*)=O] | (319) |
| 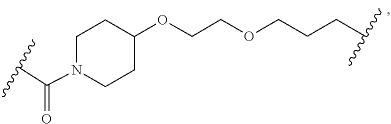 [SMILES: *CCCOCCOC1CCN(C(*)=O)CC1] | (320) |
| 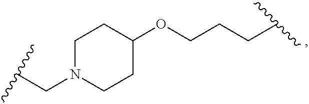 [SMILES: *CCCOC1CCN(C*)CC1] | (321) |
| 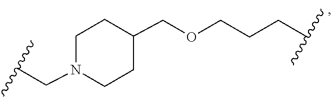 [SMILES: *CCCOCC1CCN(C*)CC1] | (322) |
| 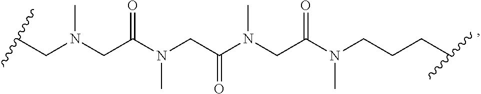 [SMILES: *CCCN(C)C(=O)CN(C)C(=O)CN(C)C(=O)CN(C)C*] | (323) |
| 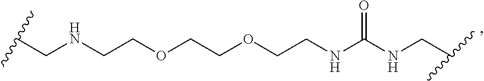 [SMILES: *CNCCOCCOCCNC(=O)NC*] | (324) |
| 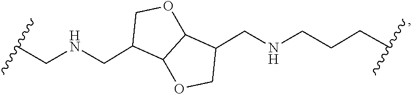 [SMILES: *CCCNCC1COC2C(CNC*)COC12] | (325) |
| 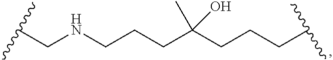 [SMILES: *CCCC(C)(O)CCCNC*] | (326) |
| 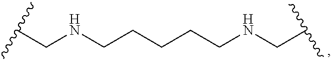 [SMILES: *CNCCCCCNC*] | (327) |
| 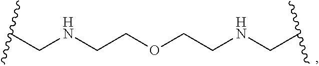 [SMILES: *CNCCOCCNC*] | (328) |
| 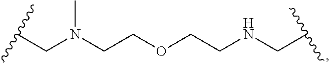 [SMILES: *CNCCOCCN(C)C*] | (329) |
| 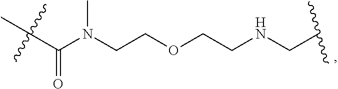 [SMILES: *CNCCOCCN(C)C(*)=O] | (330) |
| 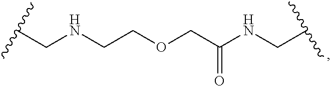 [SMILES: *CNCCOCC(=O)NC*] | (331) |
| 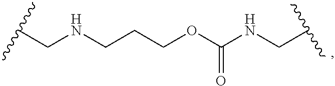 [SMILES: *CNCCCOC(=O)NC*] | (332) |
| 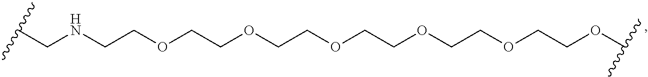 [SMILES: *CNCCOCCOCCOCCOCCOCCO*] | (333) |
| 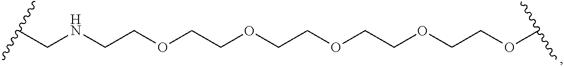 [SMILES: *CNCCOCCOCCOCCOCCO*] | (334) |
| 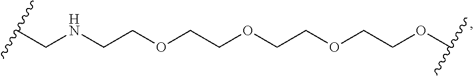 [SMILES: *CNCCOCCOCCOCCO*] | (335) |
| 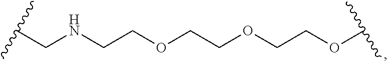 [SMILES: *CNCCOCCOCCO*] | (336) |
| 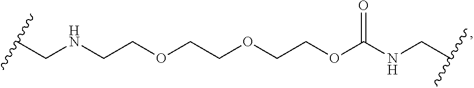 [SMILES: *CNCCOCCOCCOC(=O)NC*] | (337) |
| 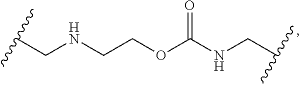 [SMILES: *CNCCOC(=O)NC*] | (338) |
| 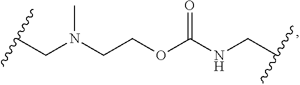 [SMILES: *CNC(=O)OCCN(C)C*] | (339) |
| 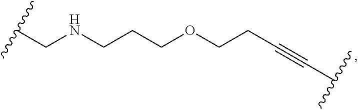 [SMILES: *C#CCCOCCCNC*] | (340) |
| 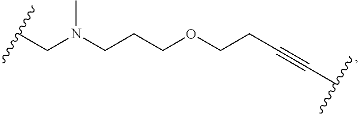 [SMILES: *C#CCCOCCCN(C)C*] | (341) |
| 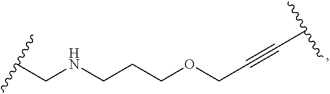 [SMILES: *C#CCOCCCNC*] | (342) |
| 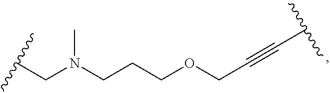 [SMILES: *C#CCOCCCN(C)C*] | (343) |
| 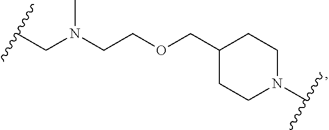 [SMILES: *CN(C)CCOCC1CCN(*)CC1] | (344) |
| 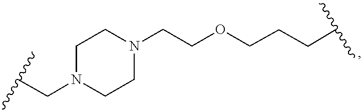 [SMILES: *CCCOCCN1CCN(C*)CC1] | (345) |
| 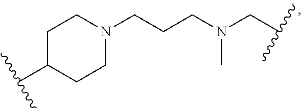 [SMILES: *CN(C)CCCN1CCC(*)CC1] | (346) |
| 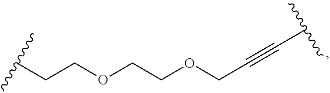 [SMILES: *C#CCOCCOCC*] | (347) |
| 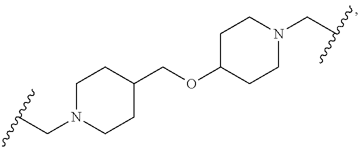 [SMILES: *CN1CCC(COC2CCN(C*)CC2)CC1] | (348) |
| 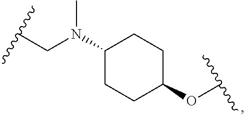 [SMILES: *CN(C)[C@H]1CC[C@H](O*)CC1] | (349) |
| 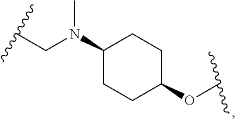 [SMILES: *CN(C)[C@H]1CC[C@@H](O*)CC1] | (350) |
| 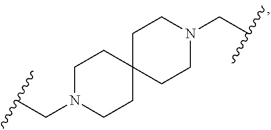 [SMILES: *CN1CCC2(CC1)CCN(C*)CC2] | (351) |
| 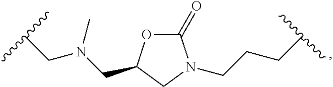 [SMILES: *CCCN1C[C@@H](CN(C)C*)OC1=O] | (352) |
| 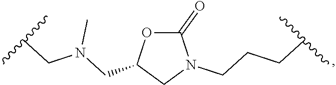 [SMILES: *CCCN1C[C@H](CN(C)C*)OC1=O] | (353) |
| 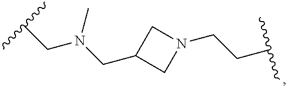 [SMILES: *CCN1CC(CN(C)C*)C1] | (354) |
| 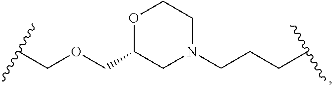 [SMILES: *CCCN1CCO[C@@H](COC*)C1] | (355) |
| 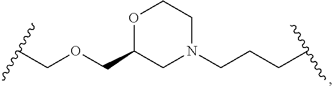 [SMILES: *CCCN1CCO[C@H](COC*)C1] | (356) |
| 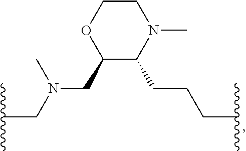 [SMILES: *CCC[C@@H]1[C@@H](CN(C)C*)OCCN1C] | (357) |
| 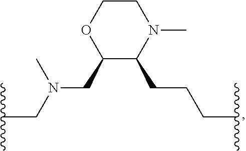 [SMILES: *CCC[C@H]1[C@@H](CN(C)C*)OCCN1C] | (358) |
| 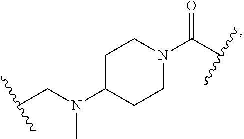 [SMILES: *CN(C)C1CCN(C(*)=O)CC1] | (359) |
| 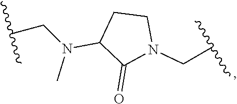 [SMILES: *CN1CCC(N(C)C*)C1=O] | (360) |
| 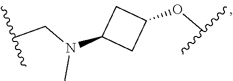 [SMILES: *CN(C)[C@H]1C[C@H](O*)C1] | (361) |
| 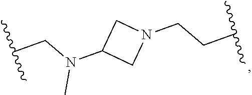 [SMILES: *CCN1CC(N(C)C*)C1] | (362) |
| 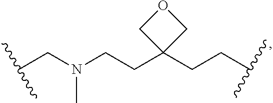 [SMILES: *CCC1(CCN(C)C*)COC1] | (363) |
| 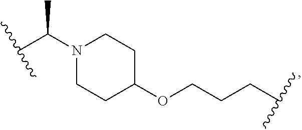 [SMILES: *CCCOC1CCN([C@@H](*)C)CC1] | (364) |
| 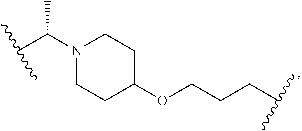 [SMILES: *CCCOC1CCN([C@H](*)C)CC1] | (365) |
| 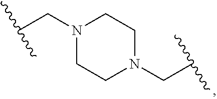 [SMILES: *CN1CCN(C*)CC1] | (366) |
| 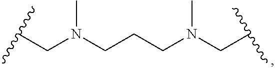 [SMILES: *CN(C)CCCN(C)C*] | (367) |
| 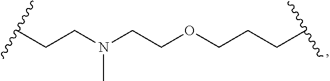 [SMILES: *CCCOCCN(C)CC*] | (368) |
| 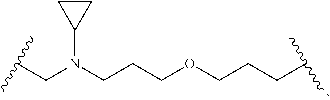 [SMILES: *CCCOCCCN(C*)C1CC1] | (369) |
| 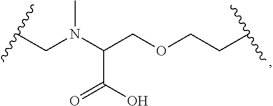 [SMILES: *CCOCC(C(=O)O)N(C)C*] | (370) |
| 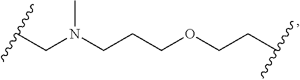 [SMILES: *CCOCCCN(C)C*] | (371) |
| 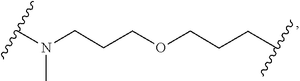 | (372) |
| 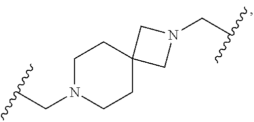 [SMILES: *CN1CCC2(CC1)CN(C*)C2] | (373) |
| 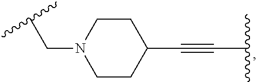 [SMILES: *C#CC1CCN(C*)CC1] | (374) |
| 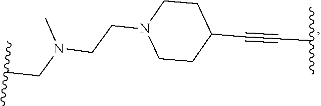 [SMILES: *C#CC1CCN(CCN(C)C*)CC1] | (375) |
| 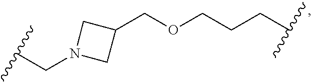 [SMILES: *CCCOCC1CN(C*)C1] | (376) |
| 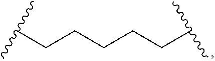 [SMILES: *CCCCC*] | (377) |
| 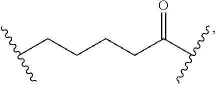 [SMILES: *CCCCC(*)=O] | (378) |
| 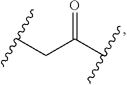 [SMILES: *CC(*)=O] | (379) |
| 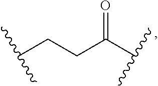 [SMILES: *CCC(*)=O] | (380) |
| 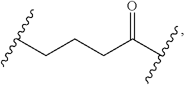 [SMILES: *CCCC(*)=O] | (381) |
| 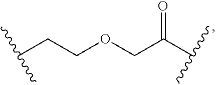 [SMILES: *CCOCC(*)=O] | (382) |
| 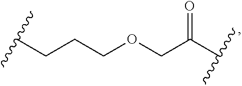 [SMILES: *CCCOCC(*)=O] | (383) |
| 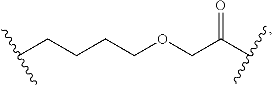 [SMILES: *CCCCOCC(*)=O] | (384) |
| 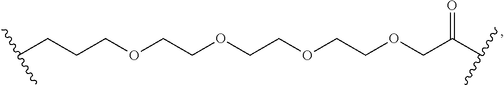 [SMILES: *CCCOCCOCCOCCOCC(*)=O] | (385) |
| 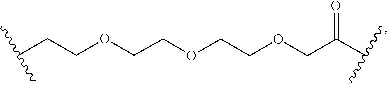 [SMILES: *CCOCCOCCOCC(*)=O] | (386) |
| 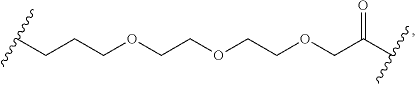 [SMILES: *CCCOCCOCCOCC(*)=O] | (387) |
| 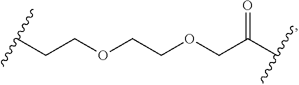 [SMILES: *CCOCCOCC(*)=O] | (388) |
| 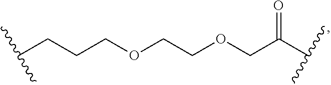 [SMILES: *CCCOCCOCC(*)=O] | (389) |
| 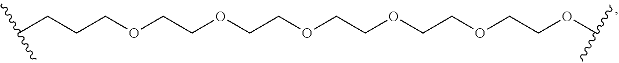 [SMILES: *CCCOCCOCCOCCOCCOCCO*] | (390) |
| 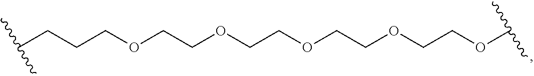 [SMILES: *CCCOCCOCCOCCOCCO*] | (391) |
| 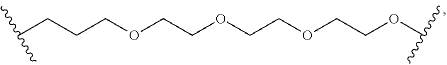 [SMILES: *CCCOCCOCCOCCO*] | (392) |
| 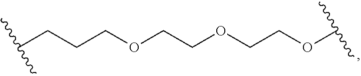 [SMILES: *CCCOCCOCCO*] | (393) |
| 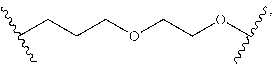 [SMILES: *CCCOCCO*] | (394) |
| 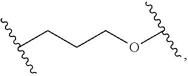 [SMILES: *CCCO*] | (395) |
| 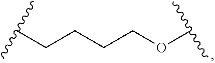 [SMILES: *CCCCO*] | (396) |
| 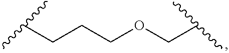 [SMILES: *CCCOC*] | (397) |
| 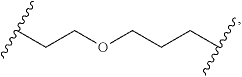 [SMILES: *CCCOCC*] | (398) |
| 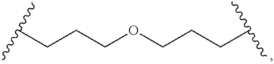 [SMILES: *CCCOCCC*] | (399) |
| 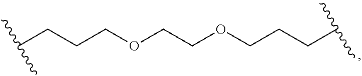 [SMILES: *CCCOCCOCCC*] | (400) |
| 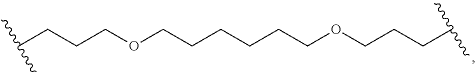 [SMILES: *CCCOCCCCCCOCCC*] | (401) |
| 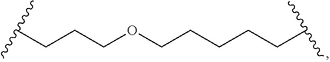 [SMILES: *CCCCCOCCC*] | (402) |
| 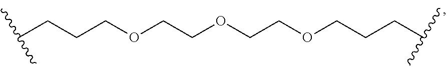 [SMILES: *CCCOCCOCCOCCC*] | (403) |
| 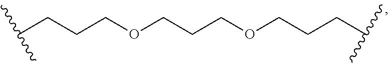 [SMILES: *CCCOCCCOCCC*] | (404) |
| 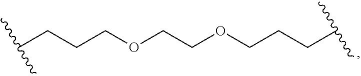 [SMILES: *CCCOCCOCCC*] | (405) |
| 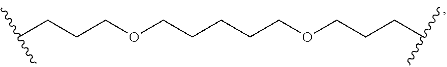 [SMILES: *CCCOCCCCCOCCC*] | (406) |
| 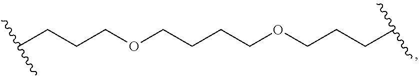 [SMILES: *CCCOCCCCOCCC*] | (407) |
| 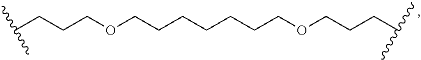 [SMILES: *CCCOCCCCCCCOCCC*] | (408) |
| 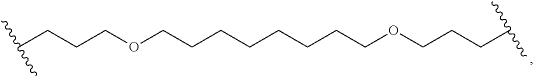 [SMILES: *CCCOCCCCCCCCOCCC*] | (409) |
| 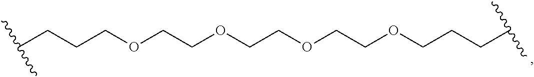 [SMILES: *CCCOCCOCCOCCOCCC*] | (410) |
| 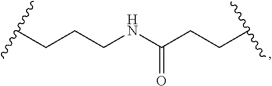 [SMILES: *CCCNC(=O)CC*] | (411) |
| 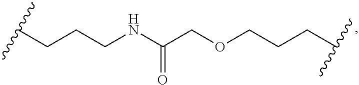 [SMILES: *CCCNC(=O)COCCC*] | (412) |
| 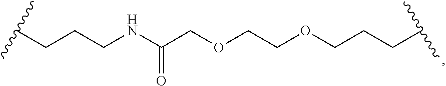 [SMILES: *CCCNC(=O)COCCOCCC*] | (413) |
| 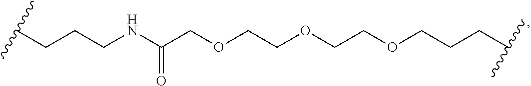 [SMILES: *CCCNC(=O)COCCOCCOCCC*] | (414) |
| 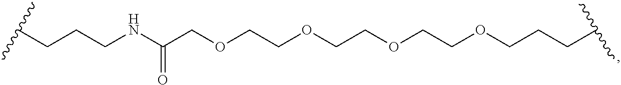 [SMILES: *CCCNC(=O)COCCOCCOCCOCCC*] | (415) |
| 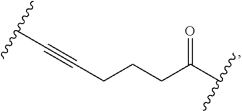 [SMILES: *C#CCCCC(*)=O] | (416) |
| 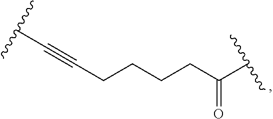 [SMILES: *C#CCCCCC(*)=O] | (417) |
| 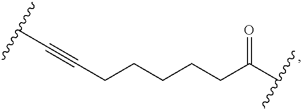 [SMILES: *C#CCCCCCC(*)=O] | (418) |
| 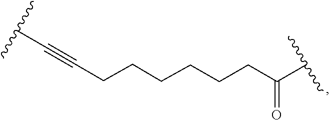 [SMILES: *C#CCCCCCCC(*)=O] | (419) |
| 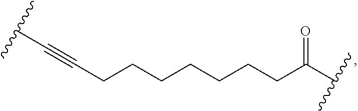 [SMILES: *C#CCCCCCCCC(*)=O] | (420) |
| 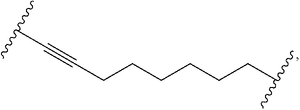 [SMILES: *C#CCCCCCC*] | (421) |
| 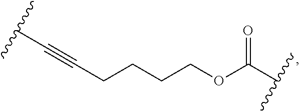 [SMILES: *C#CCCCCOC(*)=O] | (422) |
| 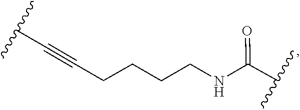 [SMILES: *C#CCCCCNC(*)=O] | (423) |
| 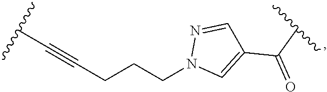 [SMILES: *C#CCCCn1cc(C(*)=O)cn1] | (424) |
| 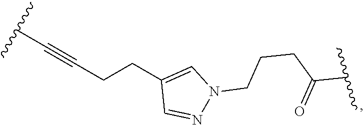 [SMILES: *C#CCCc1cnn(CCCC(*)=O)c1] | (425) |
| 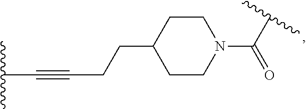 [SMILES: *C#CCCC1CCN(C(*)=O)CC1] | (426) |
| 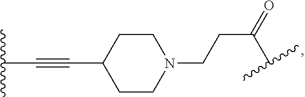 [SMILES: *C#CC1CCN(CCC(*)=O)CC1] | (427) |
| 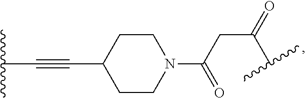 [SMILES: *C#CC1CCN(C(=O)CC(*)=O)CC1] | (428) |
| 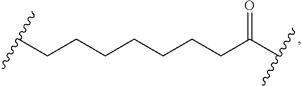 [SMILES: *CCCCCCCC(*)=O] | (429) |
| 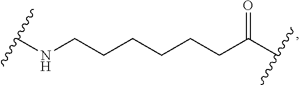 [SMILES: *NCCCCCCC(*)=O] | (430) |
| 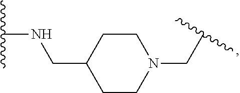 [SMILES: *CN1CCC(CN*)CC1] | (431) |
| 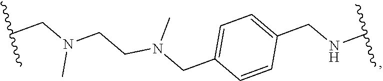 [SMILES: *CN(C)CCN(C)Cc1ccc(CN*)cc1] | (432) |
| 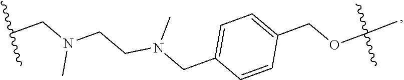 [SMILES: *CN(C)CCN(C)Cc1ccc(CO*)cc1] | (433) |
| 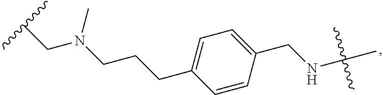 [SMILES: *CN(C)CCCc1ccc(CN*)cc1] | (434) |
| 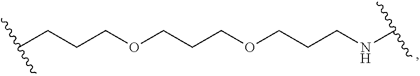 [SMILES: *CCCOCCCOCCCN*] | (435) |
| 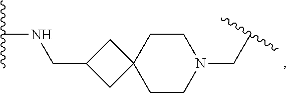 [SMILES: *CN1CCC2(CC1)CC(CN*)C2] | (436) |
| 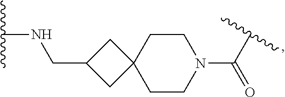 [SMILES: *NCC1CC2(CCN(C(*)=O)CC2)C1] | (437) |
| 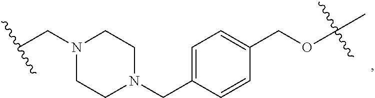 [SMILES: *CN1CCN(Cc2ccc(CO*)cc2)CC1] | (438) |
| 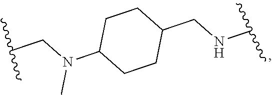 [SMILES: *CN(C)C1CCC(CN*)CC1] | (438) |
| 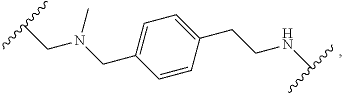 [SMILES: *CN(C)Cc1ccc(CCN*)cc1] | (439) |
| 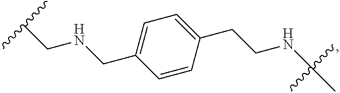 [SMILES: *CNCc1ccc(CCN*)cc1] | (440) |
| 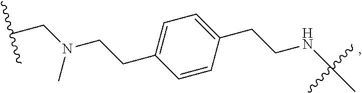 [SMILES: *CN(C)CCc1ccc(CCN*)cc1] | (441) |
| 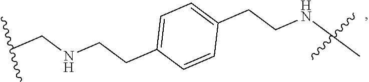 [SMILES: *CNCCc1ccc(CCN*)cc1] | (442) |
| 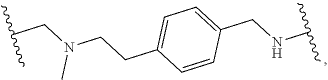 [SMILES: *CN(C)CCc1ccc(CN*)cc1] | (443) |
| 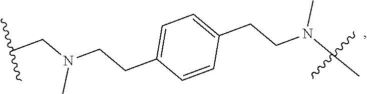 [SMILES: *CN(C)CCc1ccc(CCN(*)C)cc1] | (444) |
| 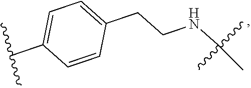 [SMILES: *NCCc1ccc(*)cc1] | (445) |
| 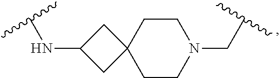 [SMILES: *CN1CCC2(CC1)CC(N*)C2] | (446) |
| 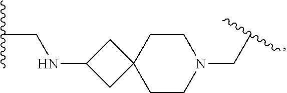 [SMILES: *CNC1CC2(CCN(C*)CC2)C1] | (447) |
| 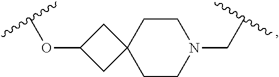 [SMILES: *CN1CCC2(CC1)CC(O*)C2] | (448) |
| 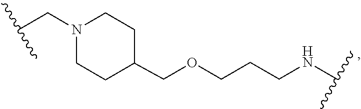 [SMILES: *CN1CCC(COCCCN*)CC1] | (449) |
| 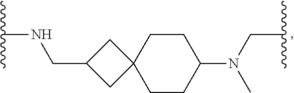 [SMILES: *CN(C)C1CCC2(CC1)CC(CN*)C2] | (450) |
| 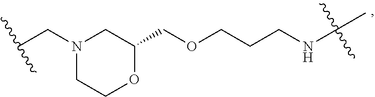 [SMILES: *CN1CCO[C@@H](COCCCN*)C1] | (451) |
| 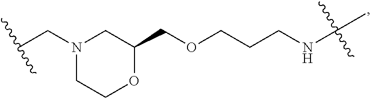 [SMILES: *CN1CCO[C@H](COCCCN*)C1] | (452) |
| 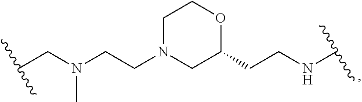 [SMILES: *CN(C)CCN1CCO[C@H](CCN*)C1] | (453) |
| 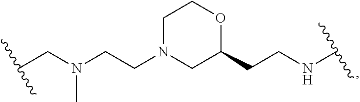 [SMILES: *CN(C)CCN1CCO[C@@H](CCN*)C1] | (454) |
| 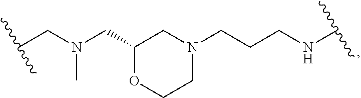 [SMILES: *CN(C)C[C@@H]1CN(CCCN*)CCO1] | (455) |
| 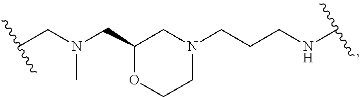 [SMILES: *CN(C)C[C@H]1CN(CCCN*)CCO1] | (456) |
| 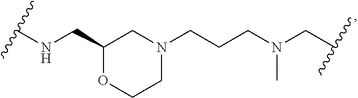 [SMILES: *CN(C)CCCN1CCO[C@@H](CN*)C1] | (457) |
| 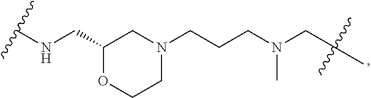 [SMILES: *CN(C)CCCN1CCO[C@H](CN*)C1] | (458) |
| 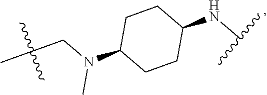 [SMILES: *CN(C)[C@H]1CC[C@@H](N*)CC1] | (459) |
| 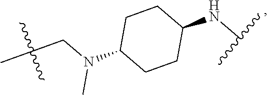 [SMILES: *CN(C)[C@H]1CC[C@H](N*)CC1] | (460) |
| 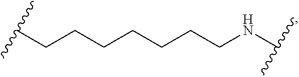 [SMILES: *CCCCCCCN*] | (461) |
| 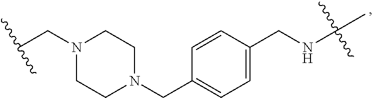 [SMILES: *CN1CCN(Cc2ccc(CN*)cc2)CC1] | (462) |
| 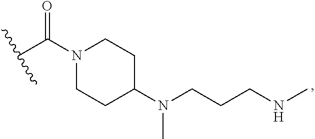 [SMILES: *C(=O)N1CCC(N(C)CCCNC)CC1 ; ","] | (463) |
| 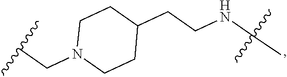 [SMILES: *CN1CCC(CCN*)CC1] | (464) |
| 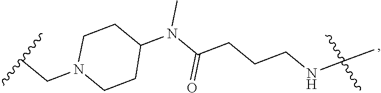 [SMILES: *CN1CCC(N(C)C(=O)CCCN*)CC1] | (465) |
| 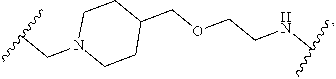 [SMILES: *CN1CCC(COCCN*)CC1] | (466) |
| 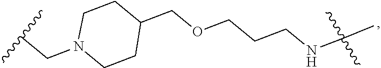 [SMILES: *CN1CCC(COCCCN*)CC1] | (467) |
| 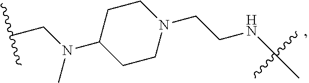 [SMILES: *CN(C)C1CCN(CCN*)CC1] | (468) |
| 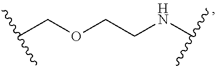 [SMILES: *COCCN*] | (469) |
| 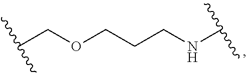 [SMILES: *COCCCN*] | (470) |
| 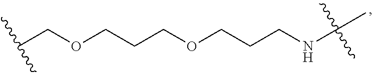 [SMILES: *COCCCOCCCN*] | (471) |
| 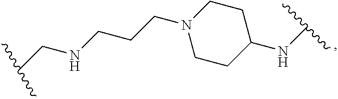 [SMILES: *CNCCCN1CCC(N*)CC1] | (472) |
| 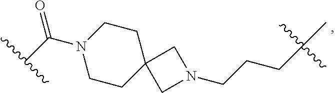 [SMILES: *CCCN1CC2(CCN(C(*)=O)CC2)C1] | (473) |
| 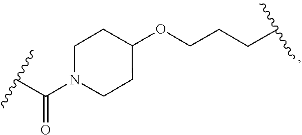 [SMILES: *CCCOC1CCN(C(*)=O)CC1] | (474) |
| 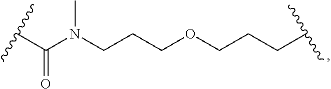 [SMILES: *CCCOCCCN(C)C(*)=O] | (475) |
| 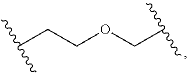 [SMILES: *CCOC*] | (476) |
| 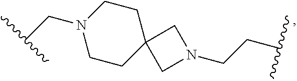 [SMILES: *CCN1CC2(CCN(C*)CC2)C1] | (477) |
| 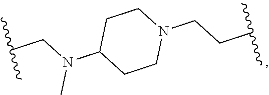 [SMILES: *CCN1CCC(N(C)C*)CC1] | (478) |
| 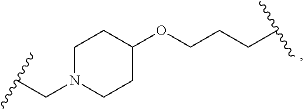 [SMILES: *CCCOC1CCN(C*)CC1] | (479) |
| 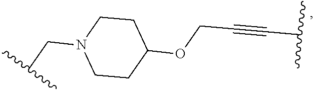 [SMILES: *C#CCOC1CCN(C*)CC1] | (480) |
| 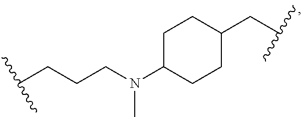 [SMILES: *CCCN(C)C1CCC(C*)CC1] | (481) |
| 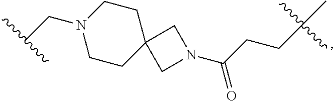 [SMILES: *CCC(=O)N1CC2(CCN(C*)CC2)C1] | (482) |
| 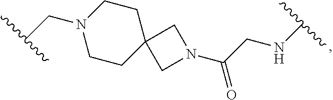 [SMILES: *CN1CCC2(CC1)CN(C(=O)CN*)C2] | (483) |
| 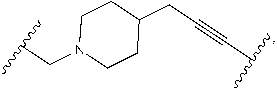 [SMILES: *C#CCC1CCN(C*)CC1] | (484) |
| 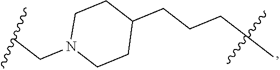 [SMILES: *CCCC1CCN(C*)CC1] | (485) |
| 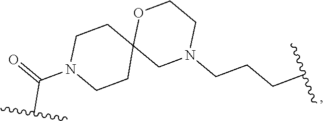 [SMILES: *CCCN1CCOC2(CCN(C(*)=O)CC2)C1] | (486) |
| 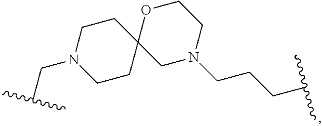 [SMILES: *CCCN1CCOC2(CCN(C*)CC2)C1] | (487) |
| 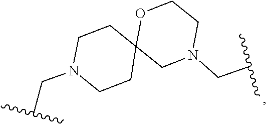 [SMILES: *CN1CCC2(CC1)CN(C*)CCO2] | (488) |
| 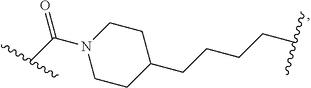 [SMILES: *CCCCC1CCN(C(*)=O)CC1] | (489) |
| 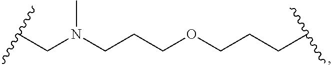 [SMILES: *CCCOCCCN(C)C*] | (490) |
| 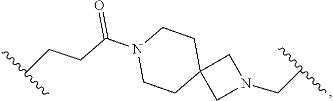 [SMILES: *CCC(=O)N1CCC2(CC1)CN(C*)C2] | (491) |
| 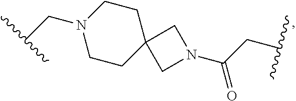 [SMILES: *CC(=O)N1CC2(CCN(C*)CC2)C1] | (492) |
| 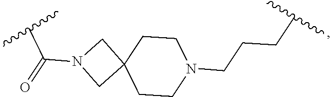 [SMILES: *CCCN1CCC2(CC1)CN(C(*)=O)C2] | (493) |
| 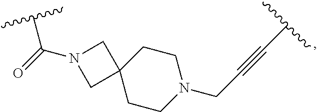 [SMILES: *C#CCN1CCC2(CC1)CN(C(*)=O)C2] | (494) |
| 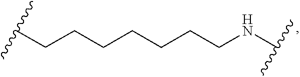 [SMILES: *CCCCCCCN*] | (495) |
| 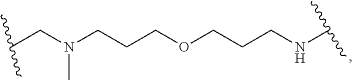 [SMILES: *CN(C)CCCOCCCN*] | (496) |
| 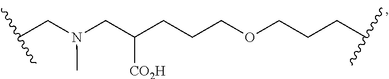 [SMILES: *CCCOCCCC(CN(C)C*)C(=O)O] | (497) |
| 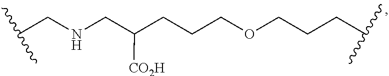 [SMILES: *CCCOCCCC(CNC*)C(=O)O] | (498) |
| 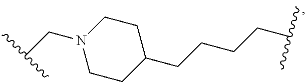 [SMILES: *CCCCC1CCN(C*)CC1] | (499) |
| 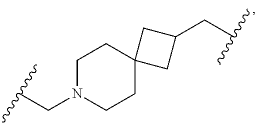 [SMILES: *CC1CC2(CCN(C*)CC2)C1] | (500) |
| 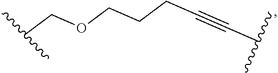 [SMILES: *C#CCCCOC*] | (501) |
| 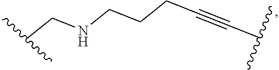 [SMILES: *C#CCCCNC*] | (502) |
| 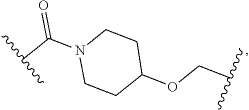 [SMILES: *COC1CCN(C(*)=O)CC1] | (503) |
| 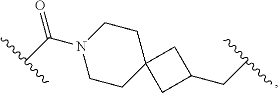 [SMILES: *CC1CC2(CCN(C(*)=O)CC2)C1] | (504) |
| 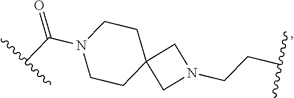 [SMILES: *CCN1CC2(CCN(C(*)=O)CC2)C1] | (505) |
| 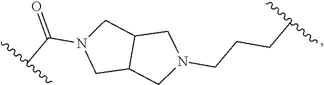 [SMILES: *CCCN1CC2CN(C(*)=O)CC2C1] | (506) |
| 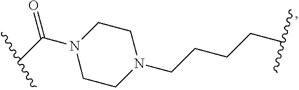 [SMILES: *CCCCN1CCN(C(*)=O)CC1] | (507) |
| 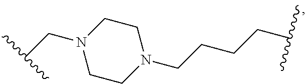 [SMILES: *CCCCN1CCN(C*)CC1] | (508) |
| 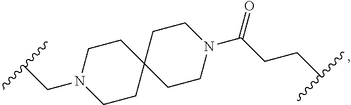 [SMILES: *CCC(=O)N1CCC2(CCN(C*)CC2)CC1] | (509) |
| 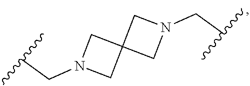 [SMILES: *CN1CC2(C1)CN(C*)C2] | (510) |
| 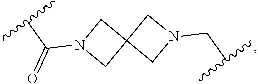 [SMILES: *CN1CC2(C1)CN(C(*)=O)C2] | (511) |
| 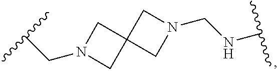 [SMILES: *CN1CC2(C1)CN(CN*)C2] | (512) |
| 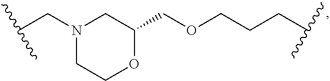 [SMILES: *CCCOC[C@H]1CN(C*)CCO1] | (513) |
| 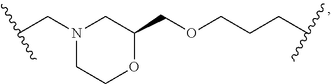 [SMILES: *CCCOC[C@@H]1CN(C*)CCO1] | (514) |
| 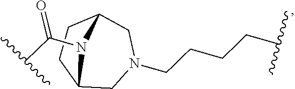 [SMILES: *CCCCN1C[C@H]2CC[C@@H](C1)N2C(*)=O] | (515) |
| 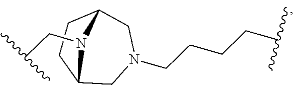 [SMILES: *CCCCN1C[C@H]2CC[C@@H](C1)N2C*] | (516) |
| 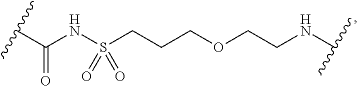 [SMILES: *NCCOCCCS(=O)(=O)NC(*)=O] | (517) |
| 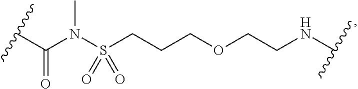 [SMILES: *NCCOCCCS(=O)(=O)N(C)C(*)=O] | (518) |
| 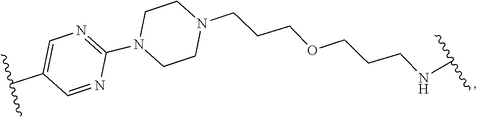 [SMILES: *NCCCOCCCN1CCN(c2ncc(*)cn2)CC1] | (519) |
| 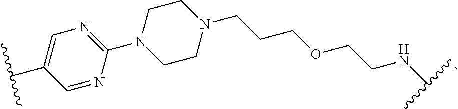 [SMILES: *NCCOCCCN1CCN(c2ncc(*)cn2)CC1] | (520) |
| 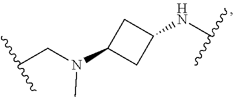 [SMILES: *CN(C)[C@H]1C[C@H](N*)C1] | (521) |
| 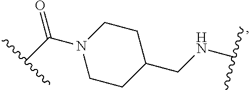 [SMILES: *NCC1CCN(C(*)=O)CC1] | (522) |
| 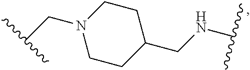 [SMILES: *CN1CCC(CN*)CC1] | (523) |
| 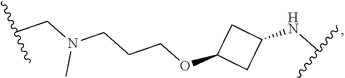 [SMILES: *CN(C)CCCO[C@H]1C[C@H](N*)C1] | (524) |
| 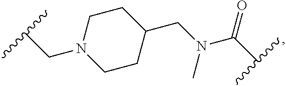 [SMILES: *CN1CCC(CN(C)C(*)=O)CC1] | (525) |
| 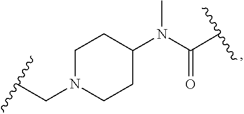 [SMILES: *CN1CCC(N(C)C(*)=O)CC1] | (526) |
| 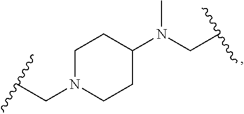 [SMILES: *CN1CCC(N(C)C*)CC1] | (527) |
| 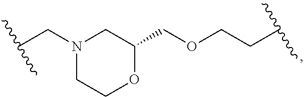 [SMILES: *CCOC[C@H]1CN(C*)CCO1] | (528) |
| 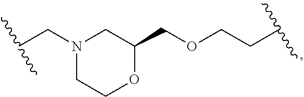 [SMILES: *CCOC[C@@H]1CN(C*)CCO1] | (529) |
| 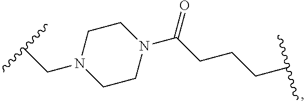 [SMILES: *CCCC(=O)N1CCN(C*)CC1] | (530) |
| 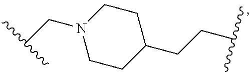 [SMILES: *CCC1CCN(C*)CC1] | (531) |
| 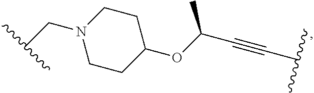 [SMILES: *C#C[C@H](C)OC1CCN(C*)CC1] | (532) |
| 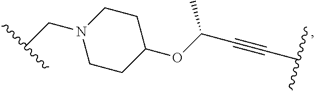 [SMILES: *C#C[C@@H](C)OC1CCN(C*)CC1] | (533) |
| 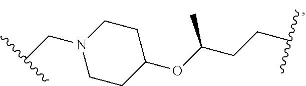 [SMILES: *CC[C@H](C)OC1CCN(C*)CC1] | (534) |
| 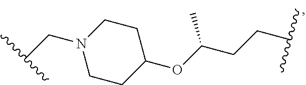 [SMILES: *CC[C@@H](C)OC1CCN(C*)CC1] | (535) |
| 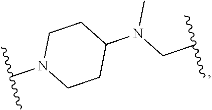 [SMILES: *CN(C)C1CCN(*)CC1] | (536) |
| 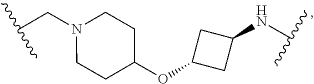 [SMILES: *CN1CCC(O[C@H]2C[C@H](N*)C2)CC1] | (537) |
| 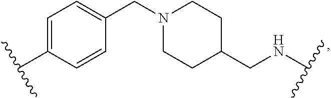 [SMILES: *NCC1CCN(Cc2ccc(*)cc2)CC1] | (538) |
| 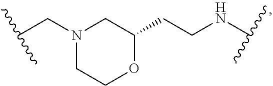 [SMILES: *CN1CCO[C@@H](CCN*)C1] | (539) |
| 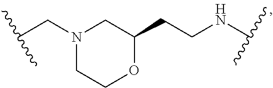 [SMILES: *CN1CCO[C@H](CCN*)C1] | (540) |
| 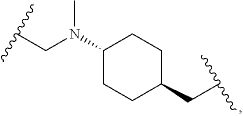 [SMILES: *CN(C)[C@H]1CC[C@H](C*)CC1] | (541) |
| 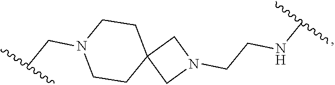 [SMILES: *CN1CCC2(CC1)CN(CCN*)C2] | (542) |
| 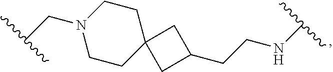 [SMILES: *CN1CCC2(CC1)CC(CCN*)C2] | (543) |
| 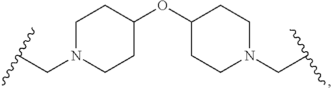 [SMILES: *CN1CCC(OC2CCN(C*)CC2)CC1] | (544) |
| 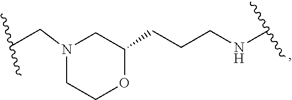 [SMILES: *CN1CCO[C@@H](CCCN*)C1] | (545) |
| 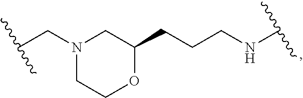 [SMILES: *CN1CCO[C@H](CCCN*)C1] | (546) |
| 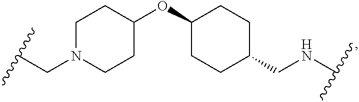 [SMILES: *CN1CCC(O[C@H]2CC[C@H](CN*)CC2)CC1] | (547) |
| 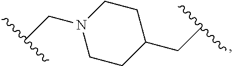 [SMILES: *CC1CCN(C*)CC1] | (548) |
| 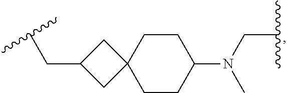 [SMILES: *CC1CC2(CCC(N(C)C*)CC2)C1] | (549) |
| 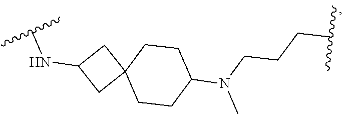 [SMILES: *CCCN(C)C1CCC2(CC1)CC(N*)C2] | (550) |
| 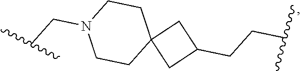 [SMILES: *CCC1CC2(CCN(C*)CC2)C1] | (551) |
| 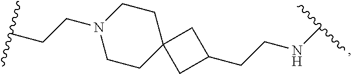 [SMILES: *CCN1CCC2(CC1)CC(CCN*)C2] | (552) |
| 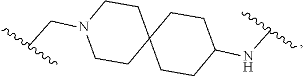 [SMILES: *CN1CCC2(CCC(N*)CC2)CC1] | (553) |
| 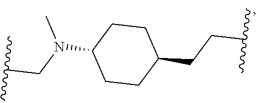 [SMILES: *CC[C@H]1CC[C@H](N(C)C*)CC1] | (554) |
| 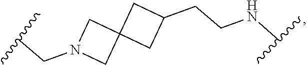 [SMILES: *CN1CC2(CC(CCN*)C2)C1] | (555) |
| 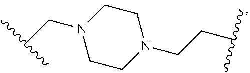 [SMILES: *CCN1CCN(C*)CC1] | (556) |
| 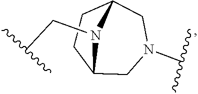 [SMILES: *CN1[C@@H]2CC[C@H]1CN(*)C2] | (557) |
| 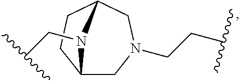 [SMILES: *CCN1C[C@H]2CC[C@@H](C1)N2C*] | (558) |
| 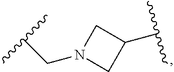 [SMILES: *CN1CC(*)C1] | (559) |
| 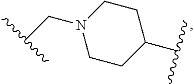 [SMILES: *CN1CCC(*)CC1] | (560) |
| 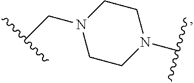 [SMILES: *CN1CCN(*)CC1] | (561) |
| 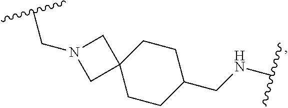 [SMILES: *CN1CC2(CCC(CN*)CC2)C1] | (562) |
| 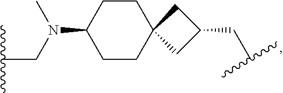 | (563) |
| 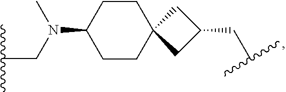 | (564) |
| 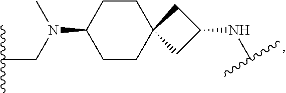 | (565) |
| 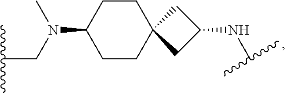 | (566) |
| 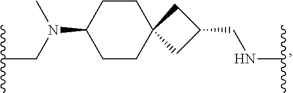 | (567) |
| 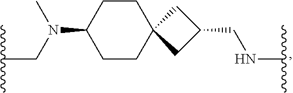 | (568) |
| 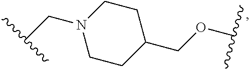 [SMILES: *CN1CCC(CO*)CC1] | (569) |
| 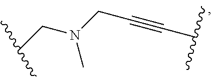 [SMILES: *C#CCN(C)C*] | (570) |
| 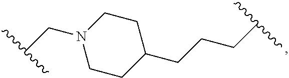 [SMILES: *CCCC1CCN(C*)CC1] | (571) |
| 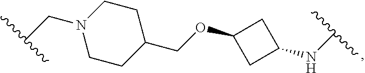 [SMILES: *CN1CCC(CO[C@H]2C[C@H](N*)C2)CC1] | (572) |
| 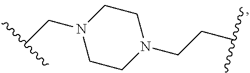 [SMILES: *CCN1CCN(C*)CC1] | (573) |
| 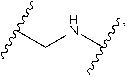 [SMILES: *CN*] | (574) |
| 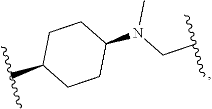 [SMILES: *CN(C)[C@H]1CC[C@@H](*)CC1] | (575) |
| 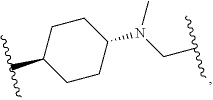 [SMILES: *CN(C)[C@H]1CC[C@H](*)CC1] | (576) |
| 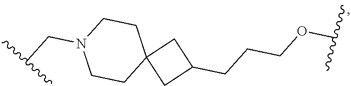 [SMILES: *CN1CCC2(CC1)CC(CCCO*)C2] | (577) |
| 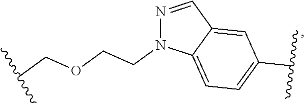 [SMILES: *COCCn1ncc2cc(*)ccc21] | (578) |
| 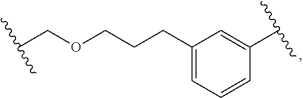 [SMILES: *COCCCc1cccc(*)c1] | (579) |
| 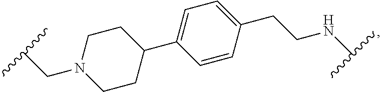 [SMILES: *CN1CCC(c2ccc(CCN*)cc2)CC1] | (580) |
| 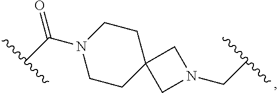 [SMILES: *CN1CC2(CCN(C(*)=O)CC2)C1] | (581) |
| 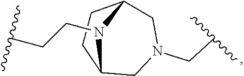 [SMILES: *CCN1[C@@H]2CC[C@H]1CN(C*)C2] | (582) |
| 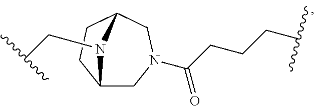 [SMILES: *CCCC(=O)N1C[C@H]2CC[C@@H](C1)N2C*] | (583) |
| 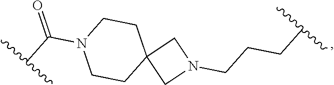 [SMILES: *CCCN1CC2(CCN(C(*)=O)CC2)C1] | (584) |
| 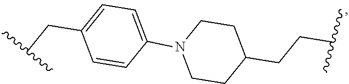 [SMILES: *CCC1CCN(c2ccc(C*)cc2)CC1] | (585) |
| 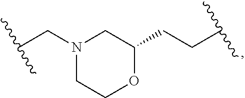 [SMILES: *CC[C@H]1CN(C*)CCO1] | (586) |
| 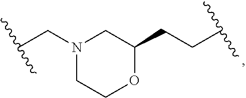 [SMILES: *CC[C@@H]1CN(C*)CCO1] | (587) |
| 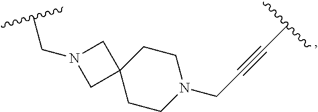 [SMILES: *C#CCN1CCC2(CC1)CN(C*)C2] | (588) |
| 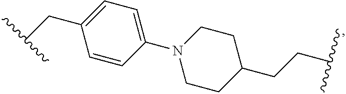 [SMILES: *CCC1CCN(c2ccc(C*)cc2)CC1] | (589) |
| 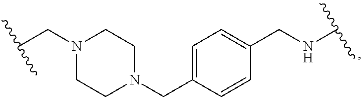 [SMILES: *CN1CCN(Cc2ccc(CN*)cc2)CC1] | (590) |
| 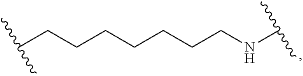 [SMILES: *CCCCCCCN*] | (591) |
| 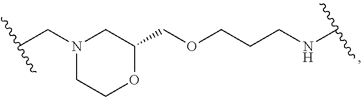 [SMILES: *CN1CCO[C@@H](COCCCN*)C1] | (592) |
| 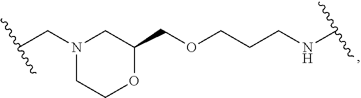 [SMILES: *CN1CCO[C@H](COCCCN*)C1] | (593) |
| 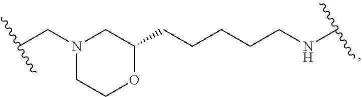 [SMILES: *CN1CCO[C@@H](CCCCCN*)C1] | (594) |
| 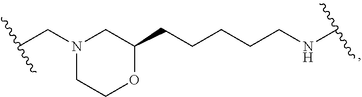 [SMILES: *CN1CCO[C@H](CCCCCN*)C1] | (595) |
| 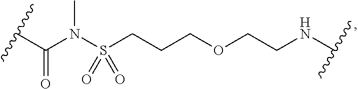 [SMILES: *NCCOCCCS(=O)(=O)N(C)C(*)=O] | (596) |
| 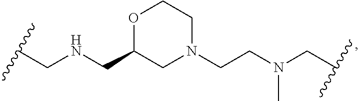 [SMILES: *CNC[C@@H]1CN(CCN(C)C*)CCO1] | (597) |
| 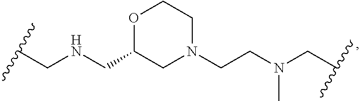 [SMILES: *CNC[C@H]1CN(CCN(C)C*)CCO1] | (598) |
| 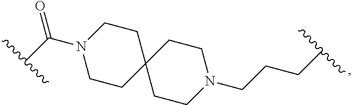 [SMILES: *CCCN1CCC2(CC1)CCN(C(*)=O)CC2] | (599) |
| 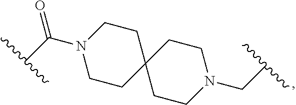 [SMILES: *CN1CCC2(CC1)CCN(C(*)=O)CC2] | (600) |
| 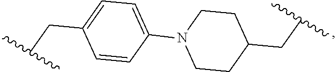 [SMILES: *Cc1ccc(N2CCC(C*)CC2)cc1] | (601) |
| 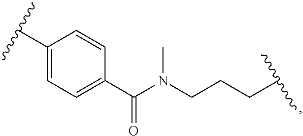 [SMILES: *CCCN(C)C(=O)c1ccc(*)cc1] | (602) |
| 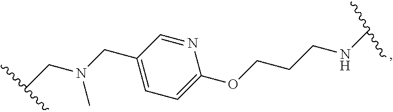 [SMILES: *CN(C)Cc1ccc(OCCCN*)nc1] | (603) |
| 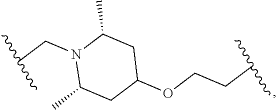 [SMILES: *CCOC1C[C@@H](C)N(C*)[C@@H](C)C1] | (604) |
| 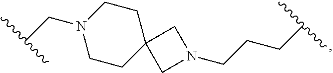 [SMILES: *CCCN1CC2(CCN(C*)CC2)C1] | (605) |
| 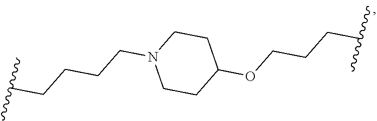 [SMILES: *CCCCN1CCC(OCCC*)CC1] | (606) |
| 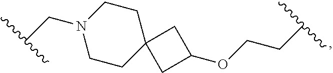 [SMILES: *CCOC1CC2(CCN(C*)CC2)C1] | (607) |
| 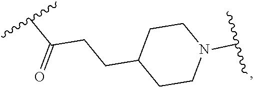 [SMILES: *C(=O)CCC1CCN(*)CC1] | (608) |
| 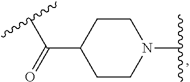 [SMILES: *C(=O)C1CCN(*)CC1] | (609) |
| 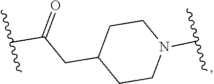 [SMILES: *C(=O)CC1CCN(*)CC1] | (610) |
| 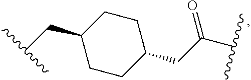 [SMILES: *C[C@H]1CC[C@H](CC(*)=O)CC1] | (611) |
| 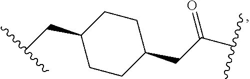 [SMILES: *C[C@H]1CC[C@@H](CC(*)=O)CC1] | (612) |
| 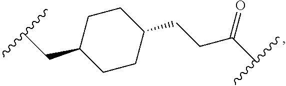 [SMILES: *C[C@H]1CC[C@H](CCC(*)=O)CC1] | (613) |
| 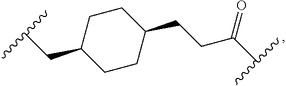 [SMILES: *C[C@H]1CC[C@@H](CCC(*)=O)CC1] | (614) |
| 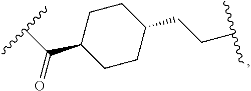 [SMILES: *CC[C@H]1CC[C@H](C(*)=O)CC1] | (615) |
| 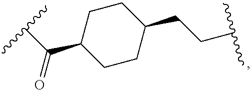 [SMILES: *CC[C@H]1CC[C@@H](C(*)=O)CC1] | (616) |
| 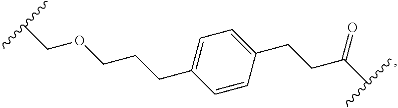 [SMILES: *COCCCc1ccc(CCC(*)=O)cc1] | (617) |
| 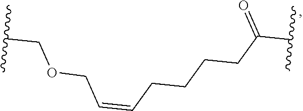 [SMILES: *COC/C=C\CCCCC(*)=O] | (618) |
| 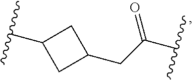 [SMILES: *C(=O)CC1CC(*)C1] | (619) |
| 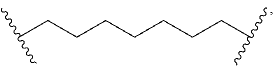 [SMILES: *CCCCCCC*] | (620) |
| 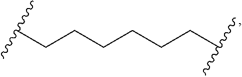 [SMILES: *CCCCCC*] | (621) |
| 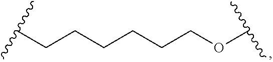 [SMILES: *CCCCCCO*] | (622) |
| 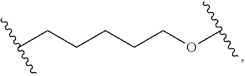 [SMILES: *CCCCCO*] | (623) |
| 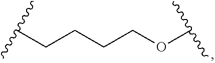 [SMILES: *CCCCO*] | (624) |
| 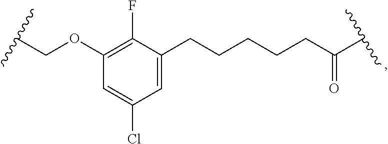 [SMILES: *COc1cc(Cl)cc(CCCCCC(*)=O)c1F] | (625) |
| 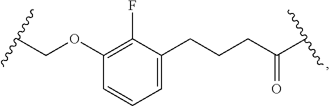 [SMILES: *COc1cccc(CCCC(*)=O)c1F] | (626) |
| 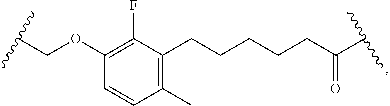 [SMILES: *COc1ccc(C)c(CCCCCC(*)=O)c1F] | (627) |
| 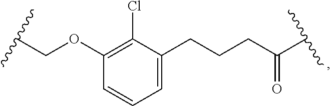 [SMILES: *COc1cccc(CCCC(*)=O)c1Cl] | (628) |
| 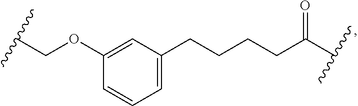 [SMILES: *COc1cccc(CCCCC(*)=O)c1] | (629) |
| 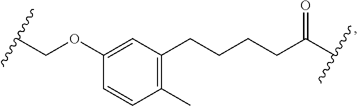 [SMILES: *COc1ccc(C)c(CCCCC(*)=O)c1] | (630) |
| 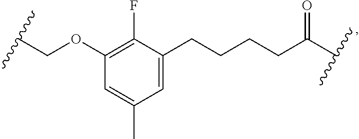 [SMILES: *COc1cc(C)cc(CCCCC(*)=O)c1F] | (631) |
| 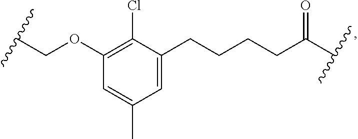 [SMILES: *COc1cc(C)cc(CCCCC(*)=O)c1Cl] | (632) |
| 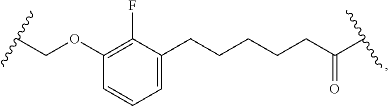 [SMILES: *COc1cccc(CCCCCC(*)=O)c1F] | (633) |
| 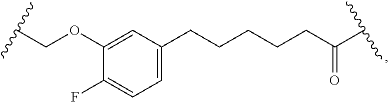 [SMILES: *COc1cc(CCCCCC(*)=O)ccc1F] | (634) |
| 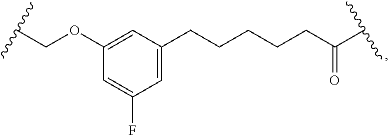 [SMILES: *COc1cc(F)cc(CCCCCC(*)=O)c1] | (635) |
| 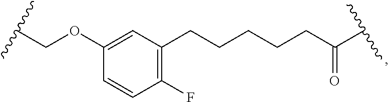 [SMILES: *COc1ccc(F)c(CCCCCC(*)=O)c1] | (636) |
| 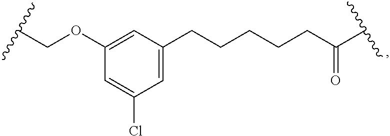 [SMILES: *COc1cc(Cl)cc(CCCCCC(*)=O)c1] | (637) |
| 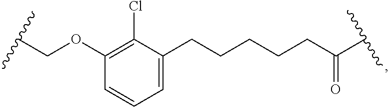 [SMILES: *COc1cccc(CCCCCC(*)=O)c1Cl] | (638) |
| 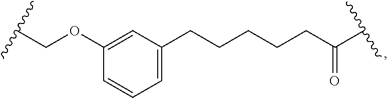 [SMILES: *COc1cccc(CCCCCC(*)=O)c1] | (639) |
| 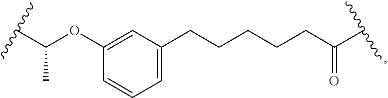 [SMILES: *C(=O)CCCCCc1cccc(O[C@@H](*)C)c1] | (640) |
| 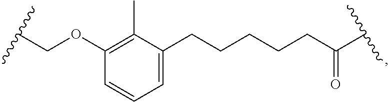 [SMILES: *COc1cccc(CCCCCC(*)=O)c1C] | (641) |
| 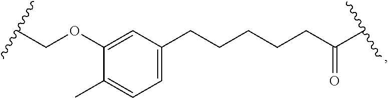 [SMILES: *COc1cc(CCCCCC(*)=O)ccc1C] | (642) |
| 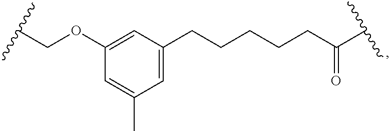 [SMILES: *COc1cc(C)cc(CCCCCC(*)=O)c1] | (643) |
| 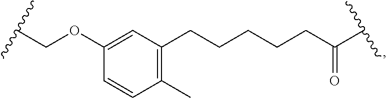 [SMILES: *COc1ccc(C)c(CCCCCC(*)=O)c1] | (644) |
| 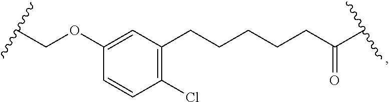 [SMILES: *COc1ccc(Cl)c(CCCCCC(*)=O)c1] | (645) |
| 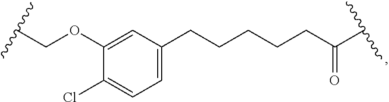 [SMILES: *COc1cc(CCCCCC(*)=O)ccc1Cl] | (646) |
| 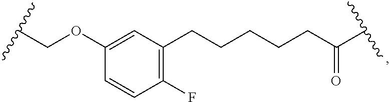 [SMILES: *COc1ccc(F)c(CCCCCC(*)=O)c1] | (647) |
| 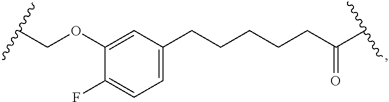 [SMILES: *COc1cc(CCCCCC(*)=O)ccc1F] | (648) |
| 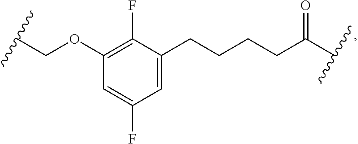 [SMILES: *COc1cc(F)cc(CCCCC(*)=O)c1F] | (649) |
| 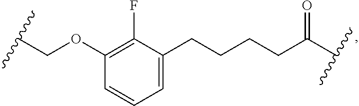 [SMILES: *COc1cccc(CCCCC(*)=O)c1F] | (650) |
| 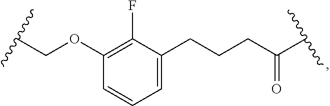 [SMILES: *COc1cccc(CCCC(*)=O)c1F] | (651) |
| 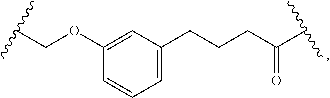 [SMILES: *COc1cccc(CCCC(*)=O)c1] | (652) |
| 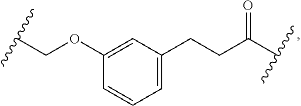 [SMILES: *COc1cccc(CCC(*)=O)c1] | (653) |
| 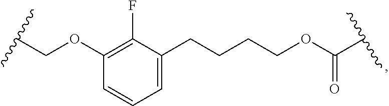 [SMILES: *COc1cccc(CCCCOC(*)=O)c1F] | (654) |
| 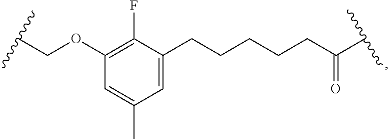 [SMILES: *COc1cc(C)cc(CCCCCC(*)=O)c1F] | (655) |
| 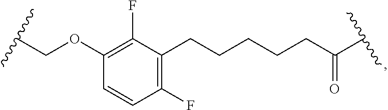 [SMILES: *COc1ccc(F)c(CCCCCC(*)=O)c1F] | (656) |
| 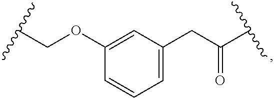 [SMILES: *COc1cccc(CC(*)=O)c1] | (657) |
| 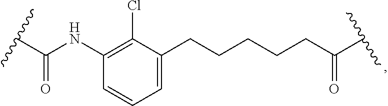 [SMILES: *C(=O)CCCCCc1cccc(NC(*)=O)c1Cl] | (658) |
| 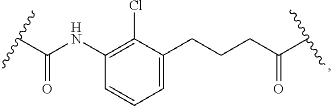 [SMILES: *C(=O)CCCc1cccc(NC(*)=O)c1Cl] | (659) |
| 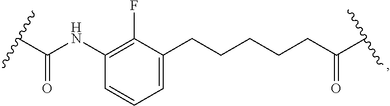 [SMILES: *C(=O)CCCCCc1cccc(NC(*)=O)c1F] | (660) |
| 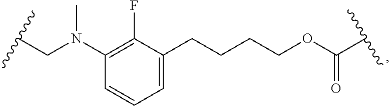 [SMILES: *CN(C)c1cccc(CCCCOC(*)=O)c1F] | (661) |
| 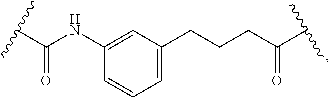 [SMILES: *C(=O)CCCc1cccc(NC(*)=O)c1] | (662) |
| 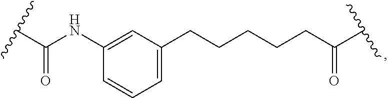 [SMILES: *C(=O)CCCCCc1cccc(NC(*)=O)c1] | (663) |
| 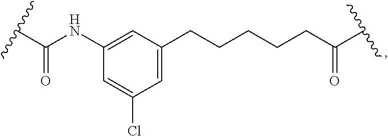 [SMILES: *C(=O)CCCCCc1cc(Cl)cc(NC(*)=O)c1] | (664) |
| 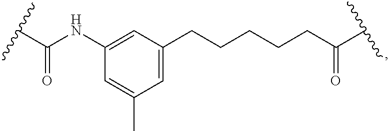 [SMILES: *C(=O)CCCCCc1cc(C)cc(NC(*)=O)c1] | (665) |
| 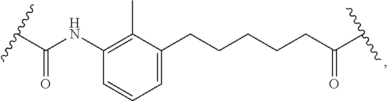 [SMILES: *C(=O)CCCCCc1cccc(NC(*)=O)c1C] | (666) |
| 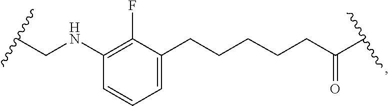 [SMILES: *CNc1cccc(CCCCCC(*)=O)c1F] | (667) |
| 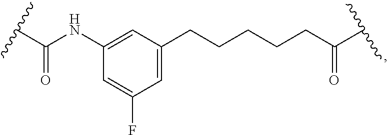 [SMILES: *C(=O)CCCCCc1cc(F)cc(NC(*)=O)c1] | (668) |
| 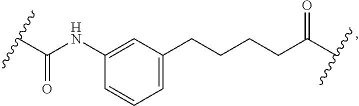 [SMILES: *C(=O)CCCCc1cccc(NC(*)=O)c1] | (669) |
| 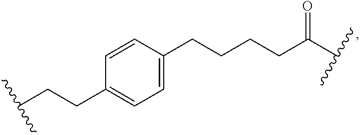 [SMILES: *CCc1ccc(CCCCC(*)=O)cc1] | (670) |
| 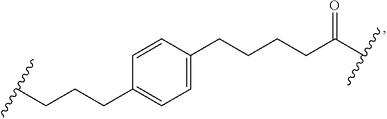 [SMILES: *CCCc1ccc(CCCCC(*)=O)cc1] | (671) |
| 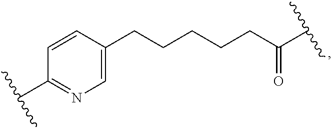 [SMILES: *C(=O)CCCCCc1ccc(*)nc1] | (672) |
| 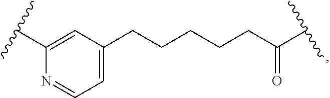 [SMILES: *C(=O)CCCCCc1ccnc(*)c1] | (673) |
| 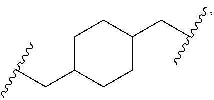 [SMILES: *CC1CCC(C*)CC1] | (674) |
| 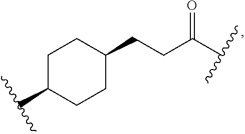 [SMILES: *C(=O)CC[C@H]1CC[C@@H](*)CC1] | (675) |
| 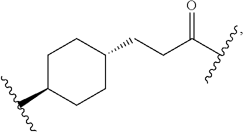 [SMILES: *C(=O)CC[C@H]1CC[C@H](*)CC1] | (676) |
| 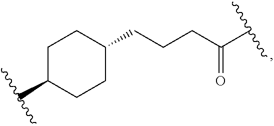 [SMILES: *C(=O)CCC[C@H]1CC[C@H](*)CC1] | (677) |
| 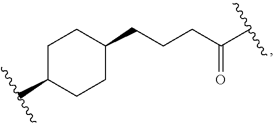 [SMILES: *C(=O)CCC[C@H]1CC[C@@H](*)CC1] | (678) |
| 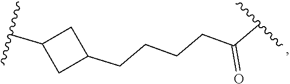 [SMILES: *C(=O)CCCCC1CC(*)C1] | (679) |
| 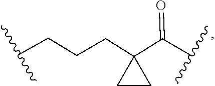 [SMILES: *CCCC1(C(*)=O)CC1] | (680) |
| or any one of the linkers described in the | |
| Linker section above. | |

In some embodiments, the present invention provides a compound or pharmaceutically acceptable salt thereof having a STAT6 binding moiety and LBM described and disclosed herein, wherein L is selected from any of those in Table B.

In some embodiments, the present invention provides a compound having a STAT6 binding moiety described and disclosed herein, a LBM set forth in Table A above, and a linker set forth in Table B above, or a pharmaceutically acceptable salt thereof. In certain embodiments, the present invention provides a compound of Formula I-a′, as a compound of Formula I-bb:

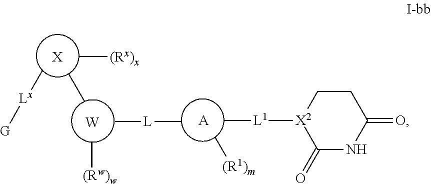

or a pharmaceutically acceptable salt thereof, wherein L, L^{1′}, L^{1′}, Ring A, Ring W, Ring X, G, R^{w}, R^{x}, R¹, X², m, w, and x are as defined above and described herein both individually and in combination.

In some embodiments, a provided compound is a compound of Formula I-bb:

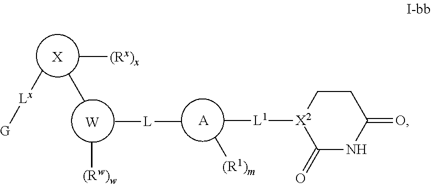

or a pharmaceutically acceptable salt thereof, wherein each of R, w, R^{X}, x, R^{y}, y, and L is as defined and described above and herein, and wherein:
X² is N or CH;
L^{1′} is a covalent bond, —C(O)—, —NR—, —O—, —S—, —S(O)₂, —NRC(O)—, or —C(O)NR—:
Ring A is phenylenyl or a 5 to 10-membered saturated or partially unsaturated monocyclic or bicyclic heterocyclylenyl or heteroarylenyl containing 1-4 heteroatoms independently selected from nitrogen, oxygen, and sulfur;
each R¹ is independently R^{A}, halogen, —CN, —NO₂, —OR, —SR, —NR₂, —SiR₃, —S(O)₂R, —S(O)₂NR₂—S(O)R, —C(O)R, —C(O)OR, —C(O)NR₂, —C(O)N(R)OR, —C(R)₂N(R)C(O)R, —C(R)₂N(R)C(O)NR₂, —OC(O)R, —OC(O)NR₂, —OP(O)R₂, —OP(O)(OR)₂, —OP(O)(OR)(NR₂), —OP(O)(NR₂)₂, —N(R)C(O)OR, —N(R)C(O)R, —N(R)C(O)NR₂, —N(R)S(O)₂R, —NP(O)R₂, —N(R)P(O)(OR)₂, —N(R)P(O)(OR)(NR₂), —N(R)P(O)(NR₂)₂, —N(R)S(O)₂R;
Ring W is a 9-membered bicyclic heteroaryl with 1-4 heteroatoms independently selected from nitrogen, oxygen, and sulfur;
Ring X is a 6-membered saturated or partially unsaturated carbocyclylenyl or heterocyclylenyl with 1-3 heteroatoms independently selected from nitrogen, oxygen, and sulfur;
L^{X} is a covalent bond or a C₁₋₅ bivalent straight or branched saturated or unsaturated hydrocarbon chain wherein 1-2 methylene units of the chain are independently and optionally replaced with —O—, —C(O)—, —C(S)—, —CR₂—, —NR—, —S—, —S(O)—, or —S(O)₂—:
G is hydrogen, halogen, or

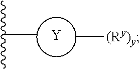

and
Ring Y is a 3- to 6-membered saturated or partially unsaturated carbocyclyl, or 4- to 6-membered monocyclic saturated or partially unsaturated heterocyclyl or heteroaryl ring with 1-4 heteroatoms independently selected from nitrogen, oxygen, or sulfur.

In some embodiments, the present invention provides a compound of formula I-bb as a compound of any one of the following formulae:

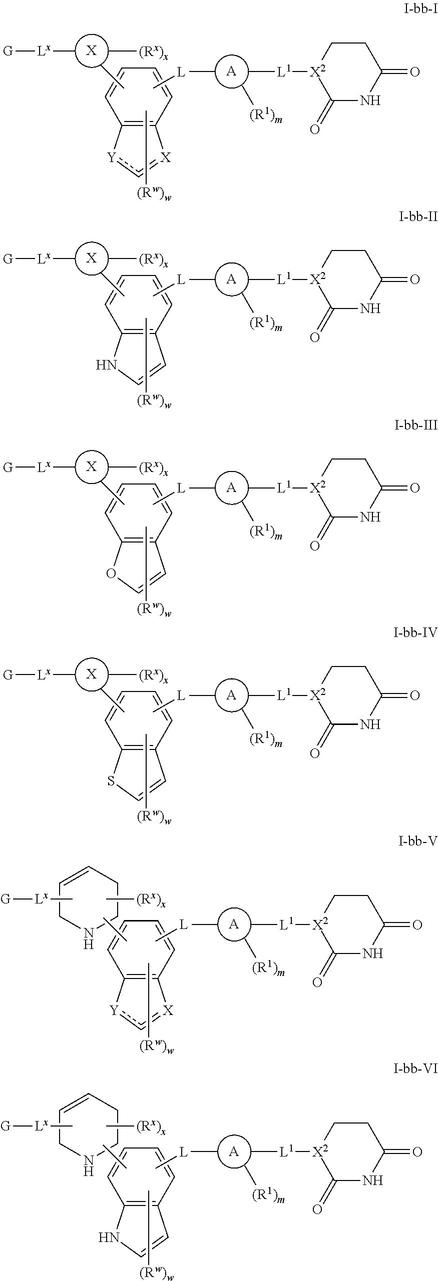

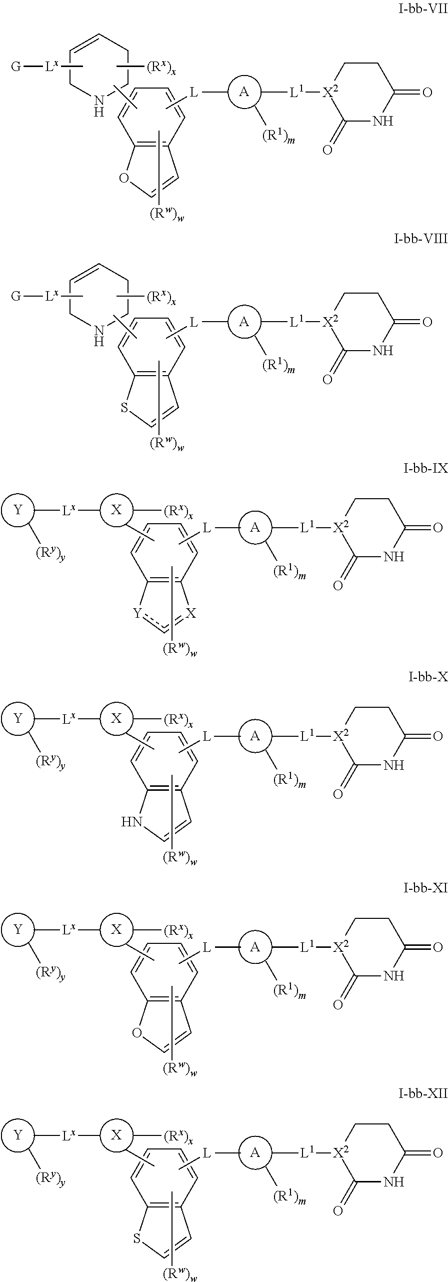

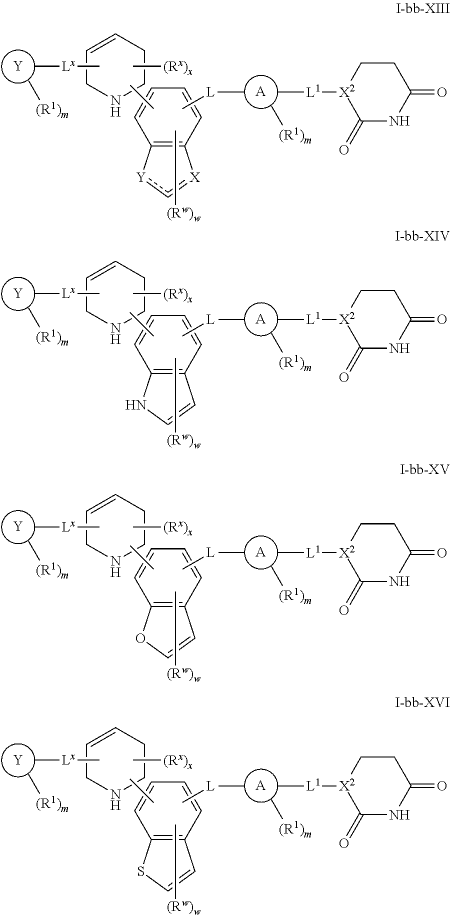

or a pharmaceutically acceptable salt thereof, wherein each variable is as defined above and described herein both individually and in combination.

In some embodiments, the present invention provides a compound of formula I-bb as a compound of any one of the following formulae:

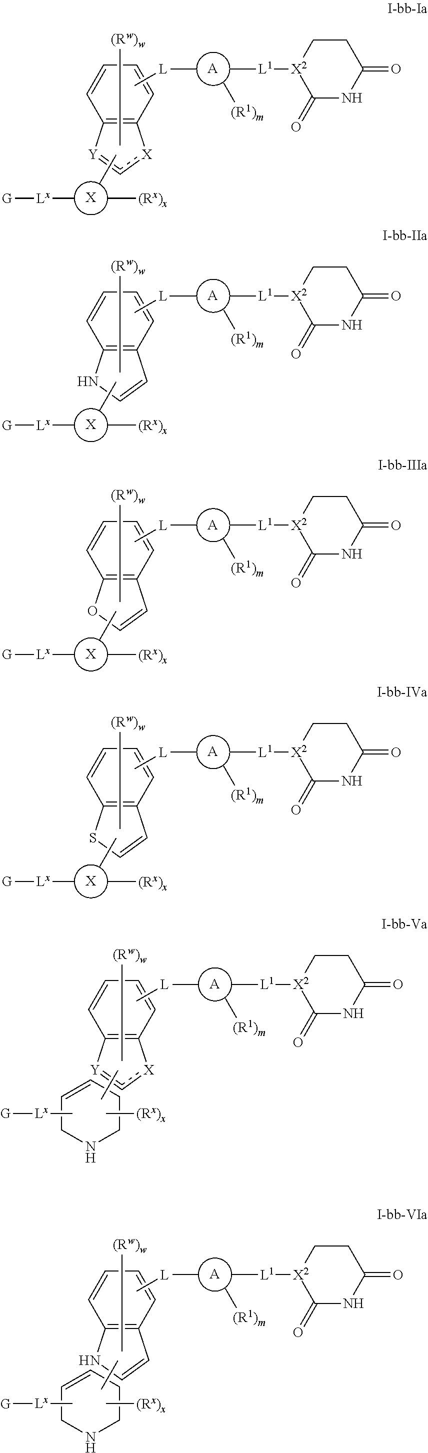

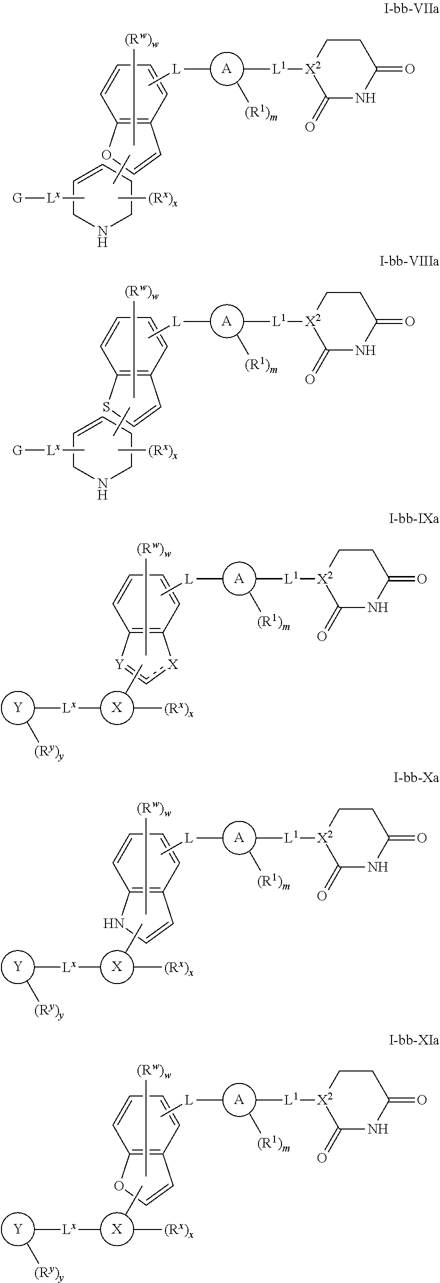

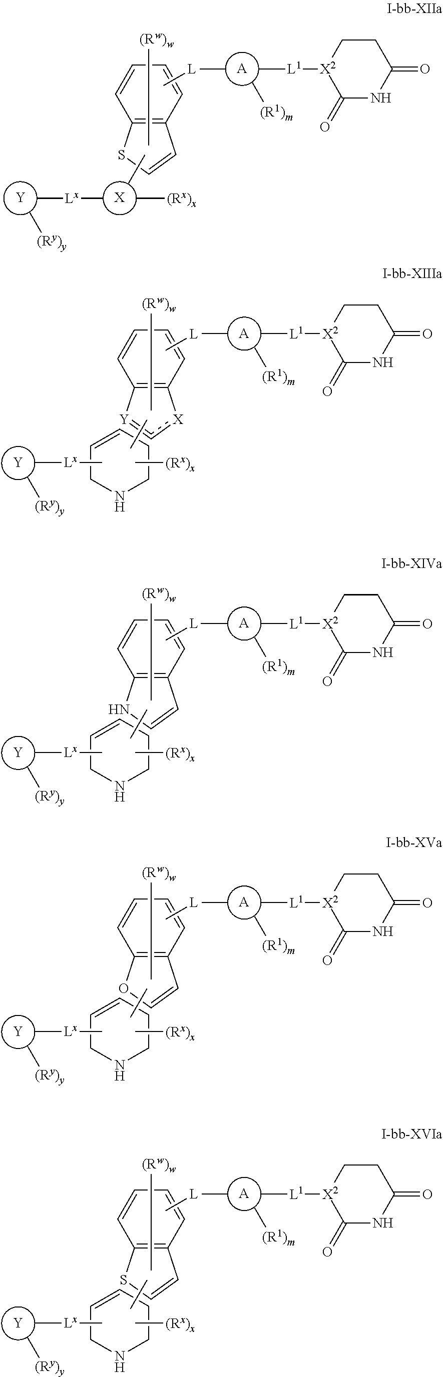

or a pharmaceutically acceptable salt thereof, wherein each variable is as defined above and described herein both individually and in combination.

In some embodiments, the present invention provides a compound of formula I-bb as a compound of any one of the following formulae:

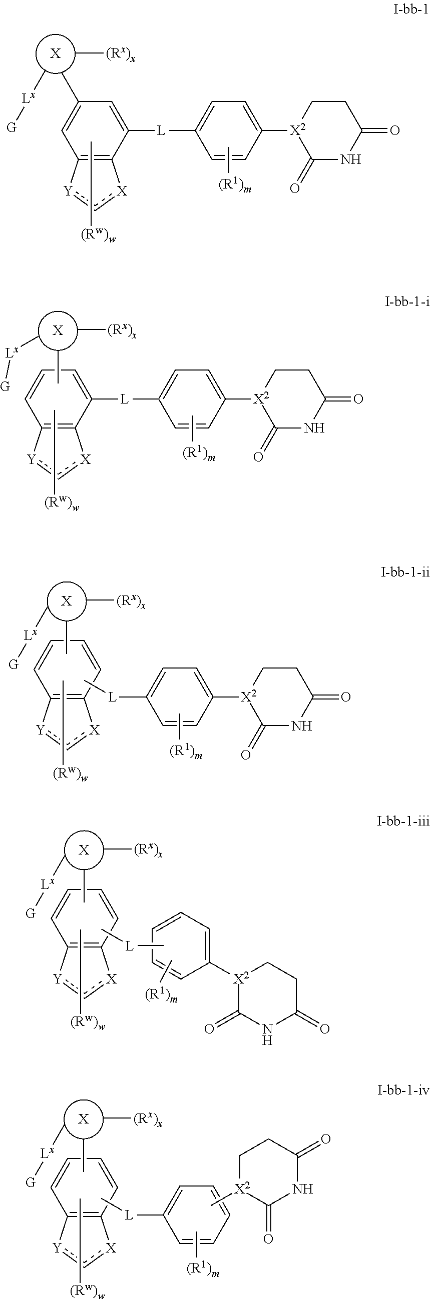

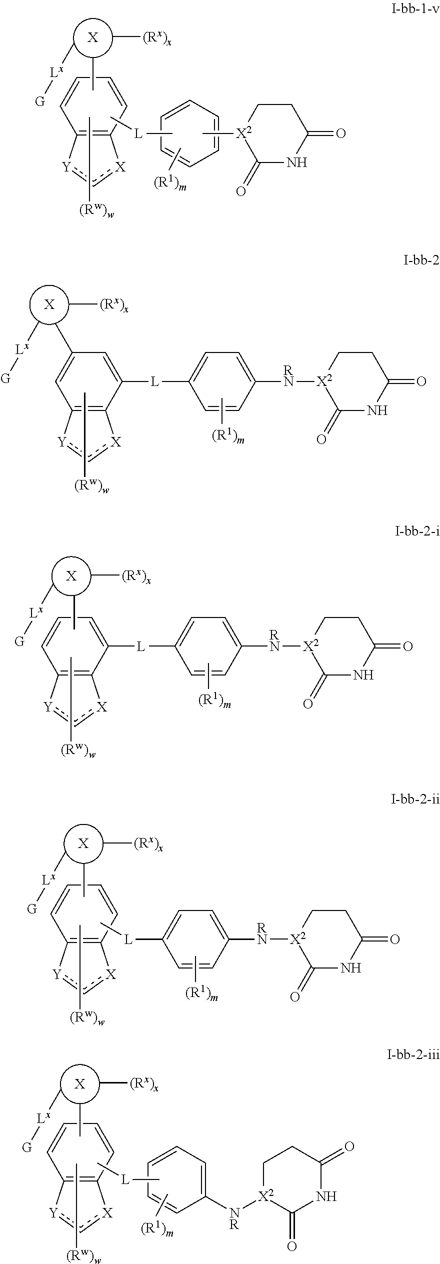

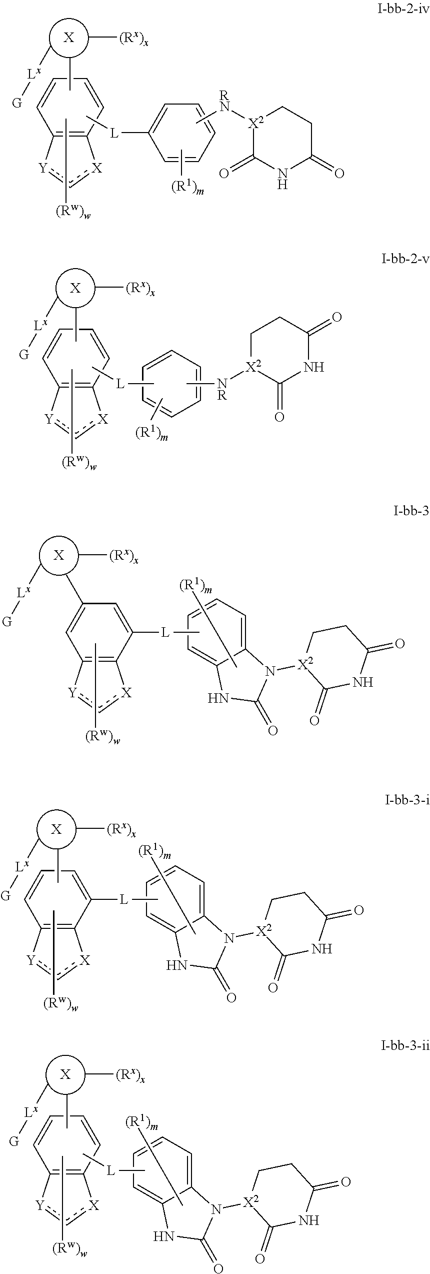

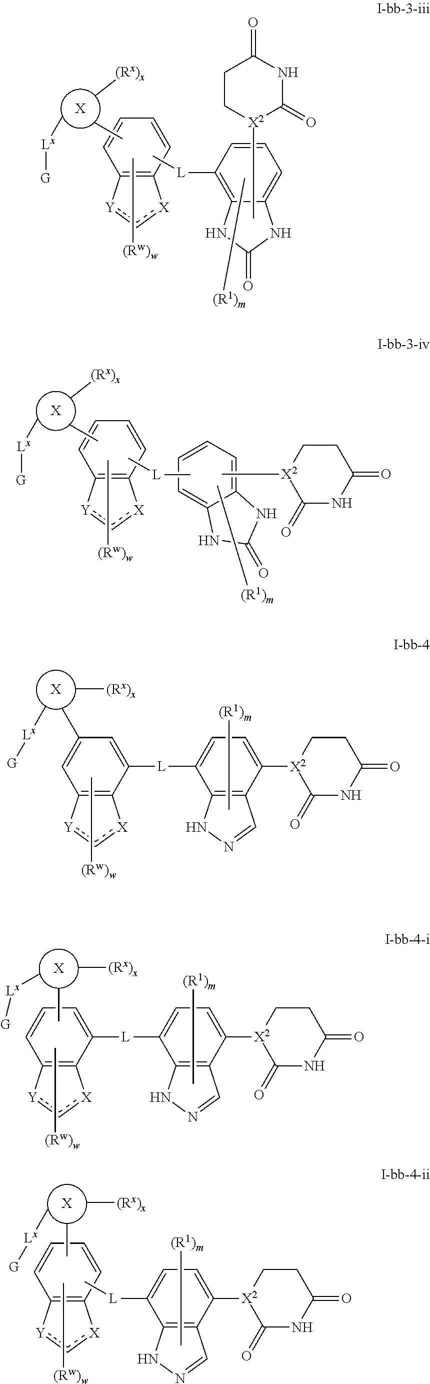

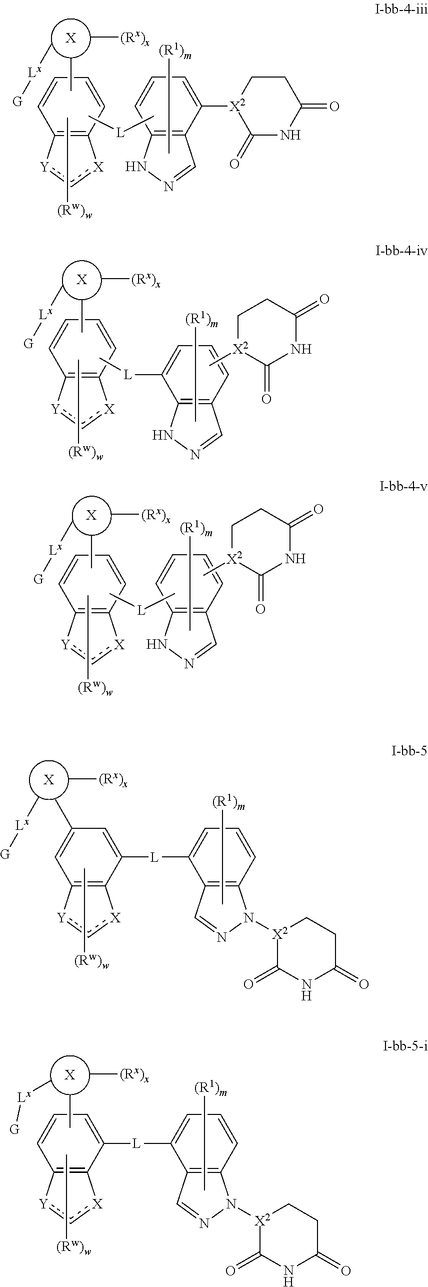

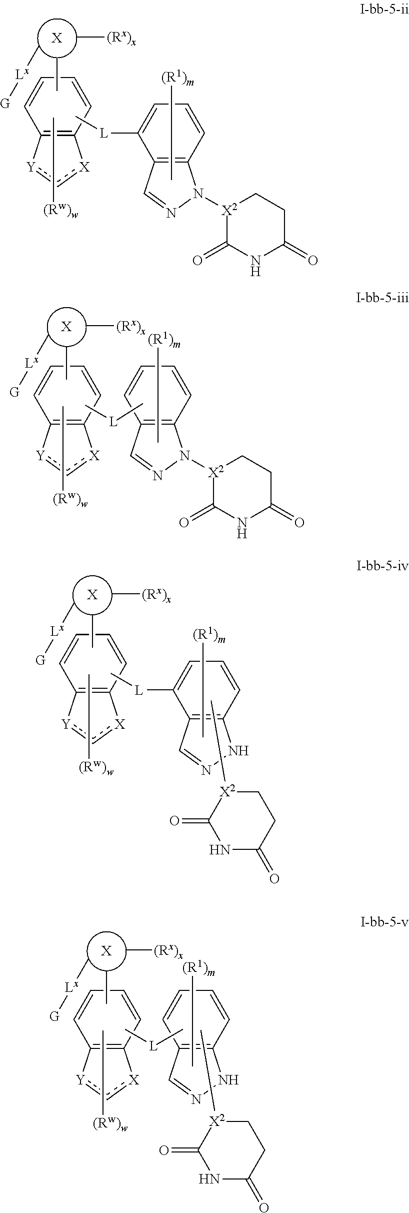

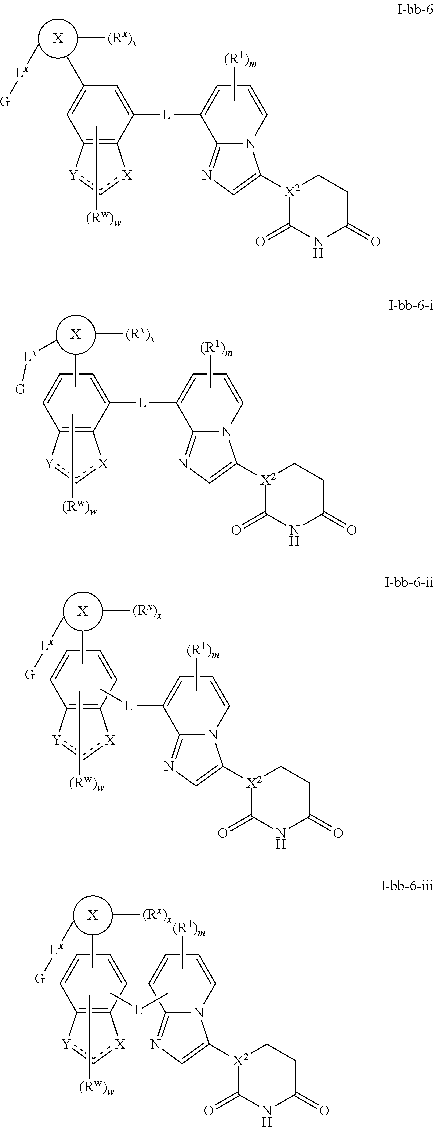

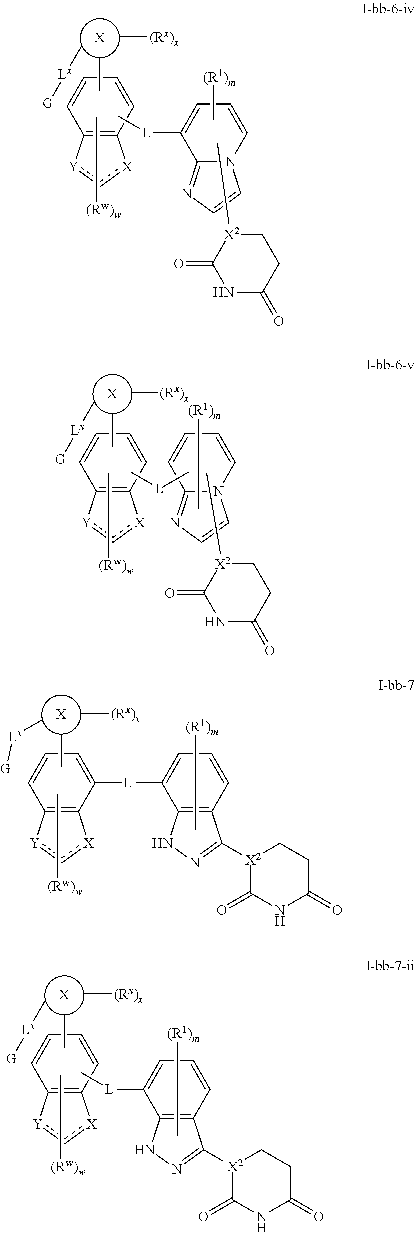

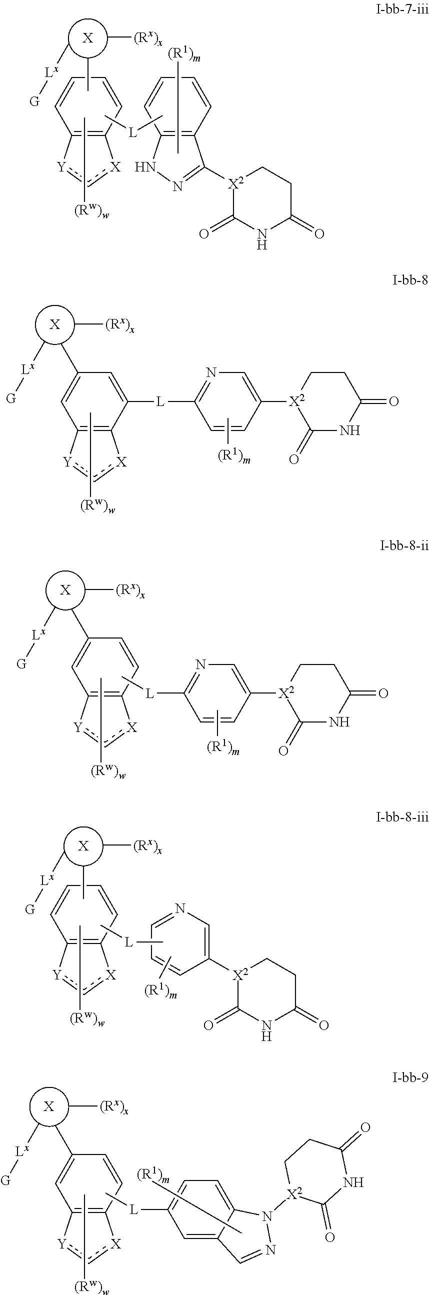

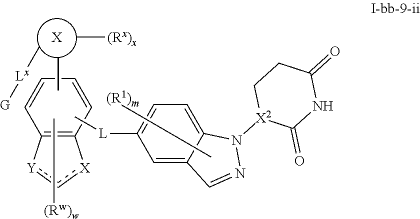

or a pharmaceutically acceptable salt thereof, wherein each variable is as defined above and described herein both individually and in combination.

In some embodiments, the present invention provides a compound of formula I-bb as a compound of any one of the following formulae:

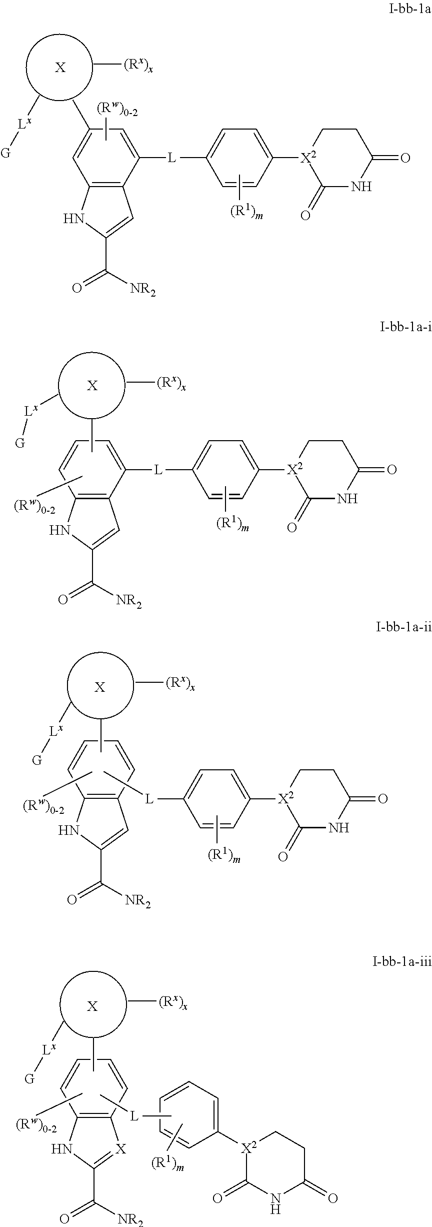

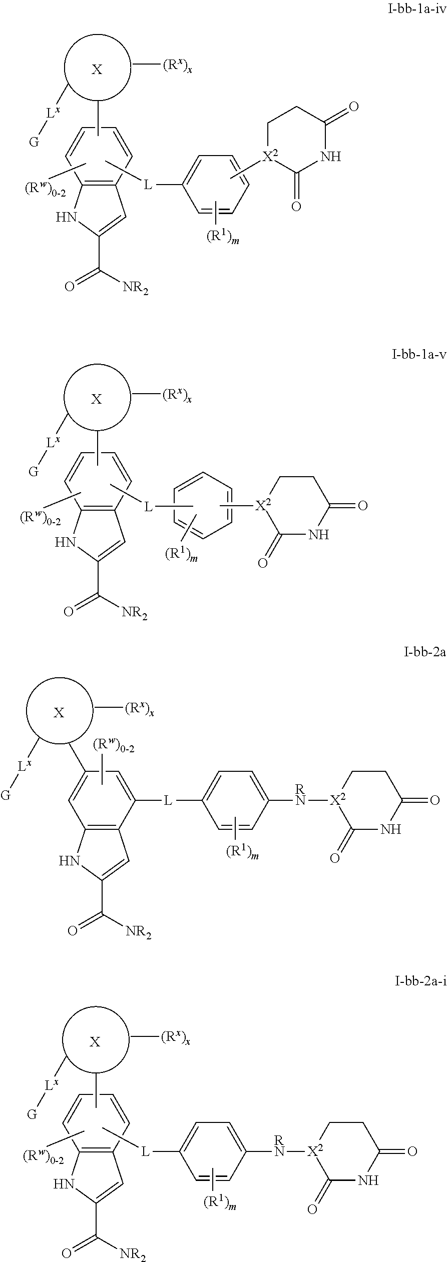

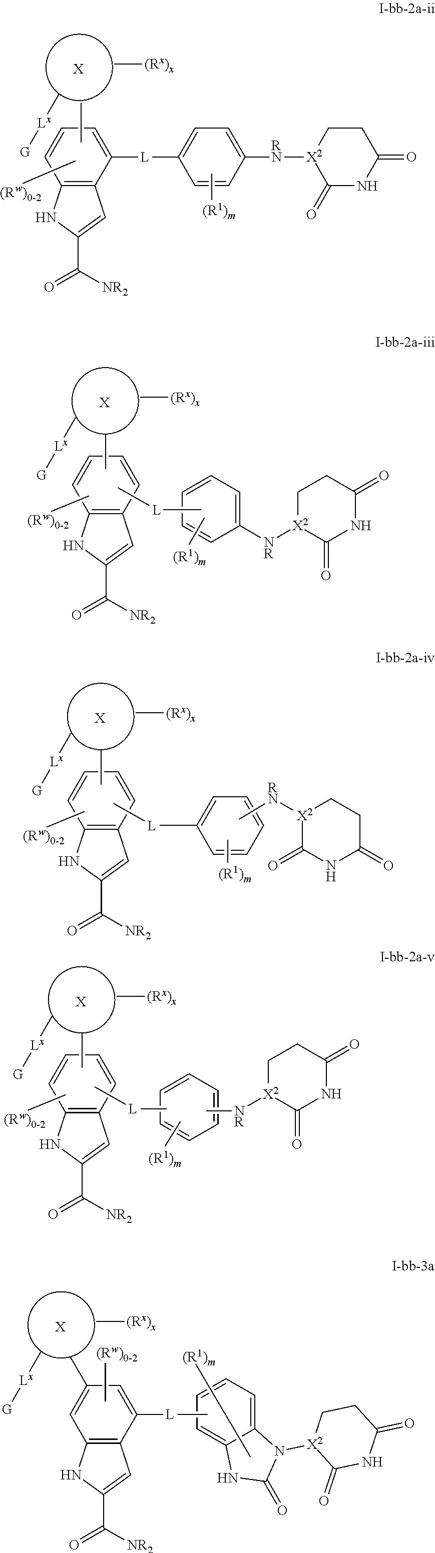

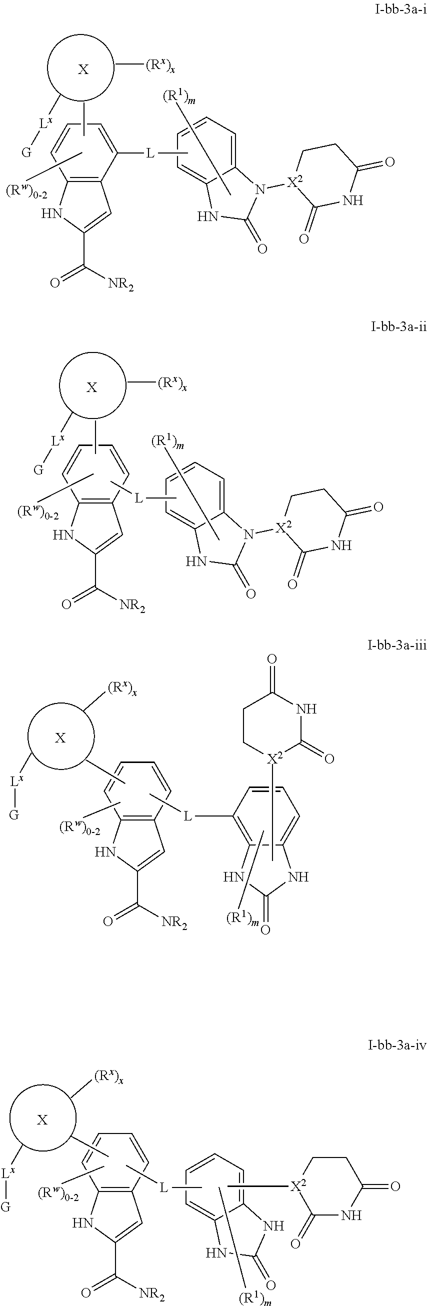

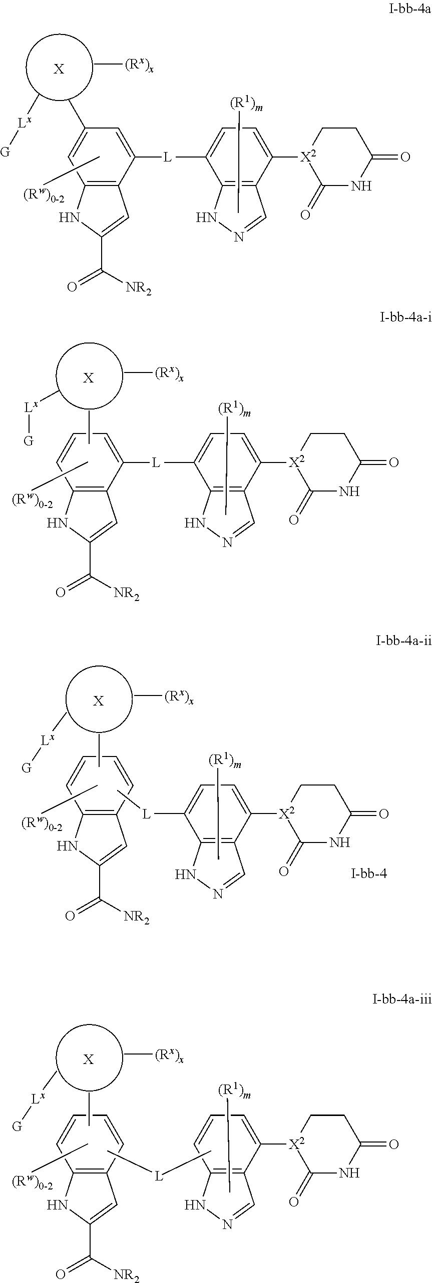

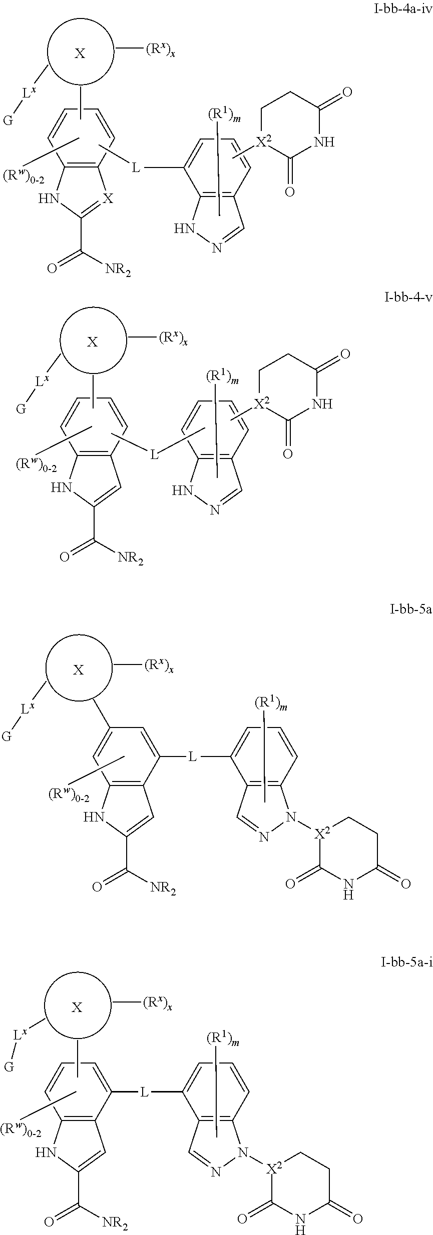

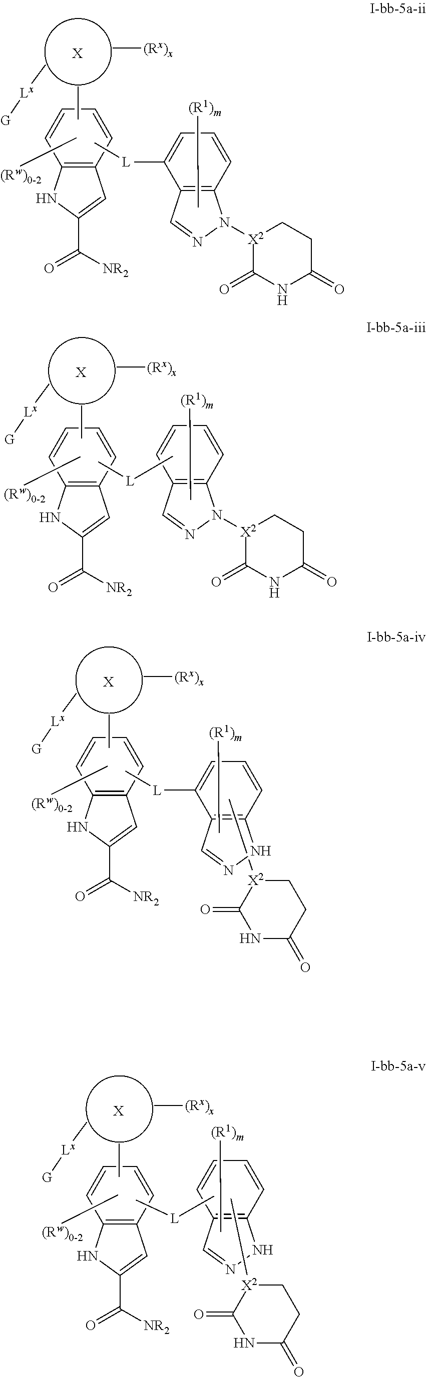

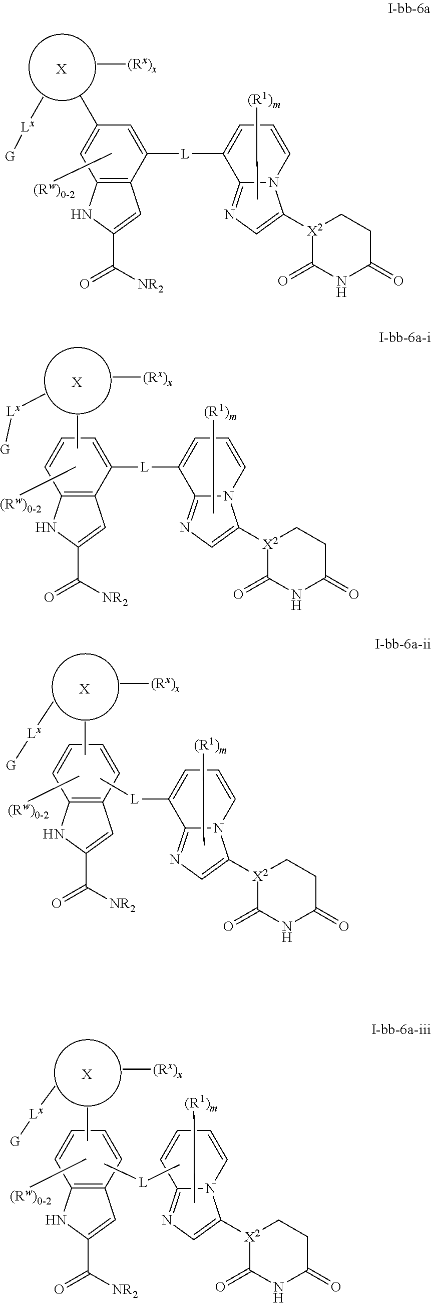

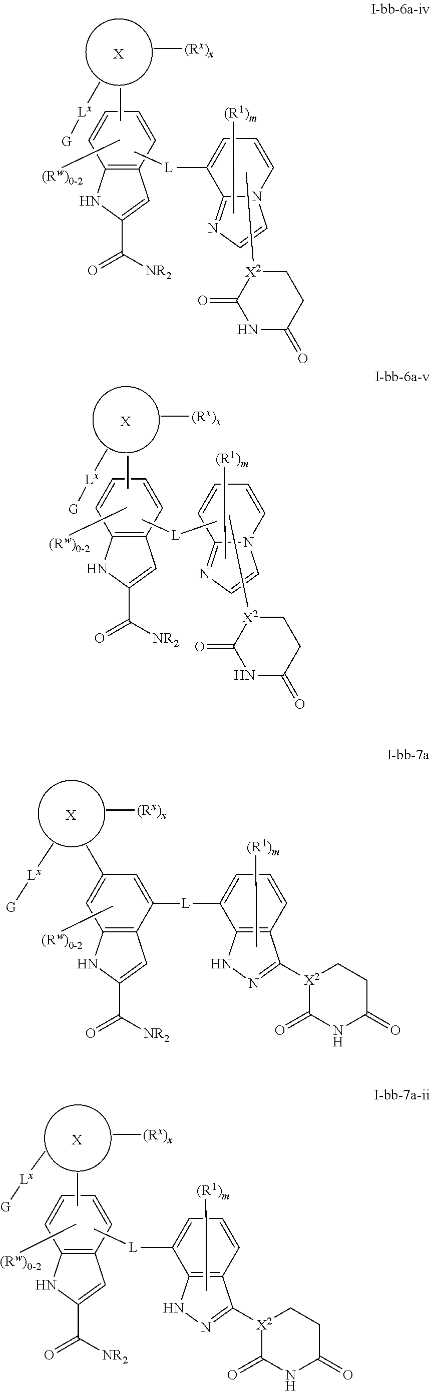

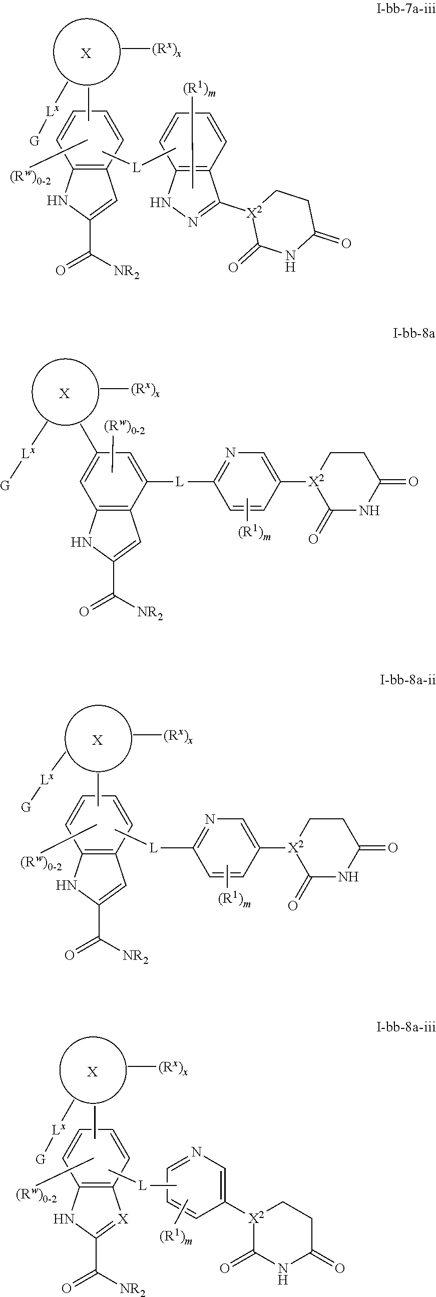

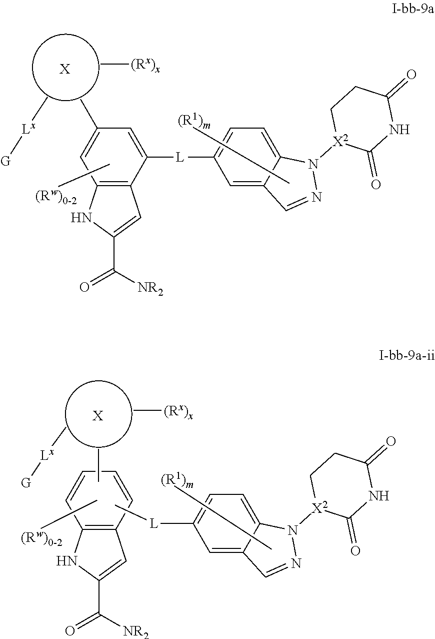

or a pharmaceutically acceptable salt thereof, wherein each variable is as defined above and described herein both individually and in combination.

In some embodiments, the present invention provides a compound of formula I-bb as a compound of any one of the following formulae:

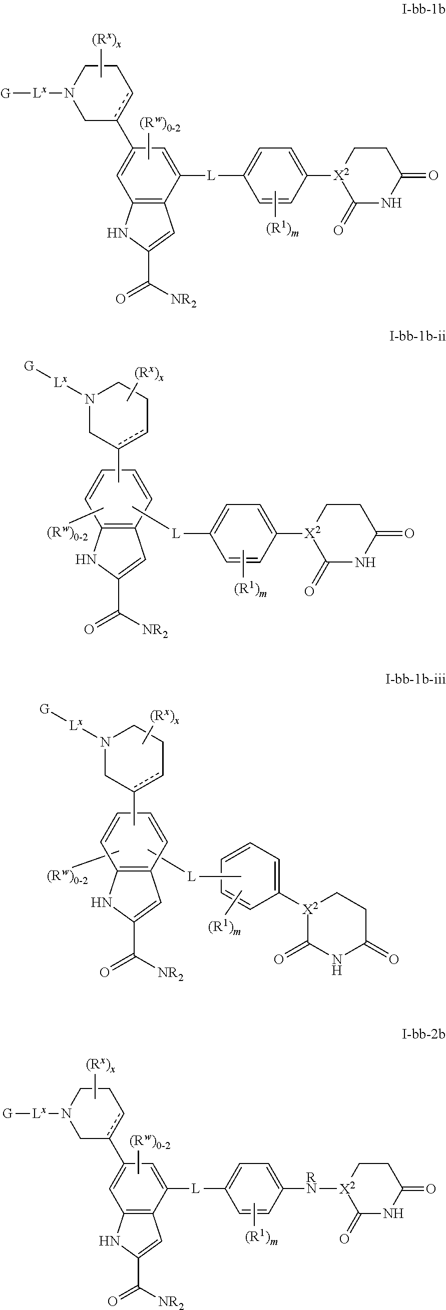

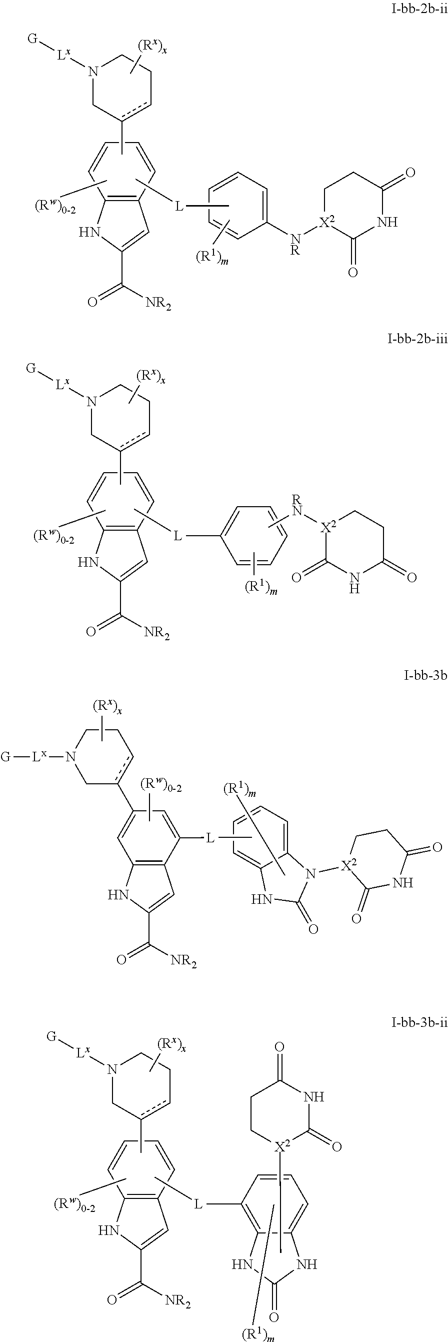

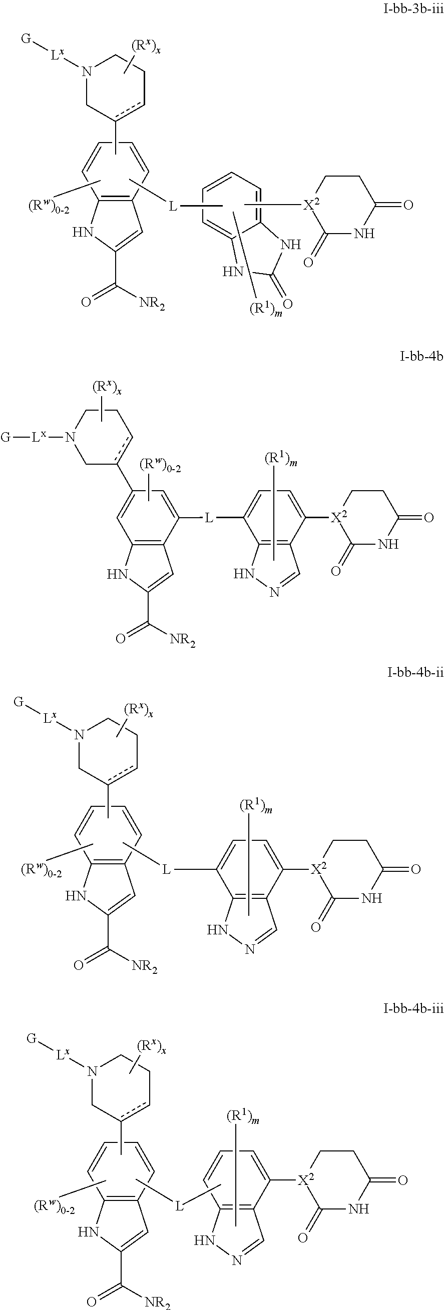

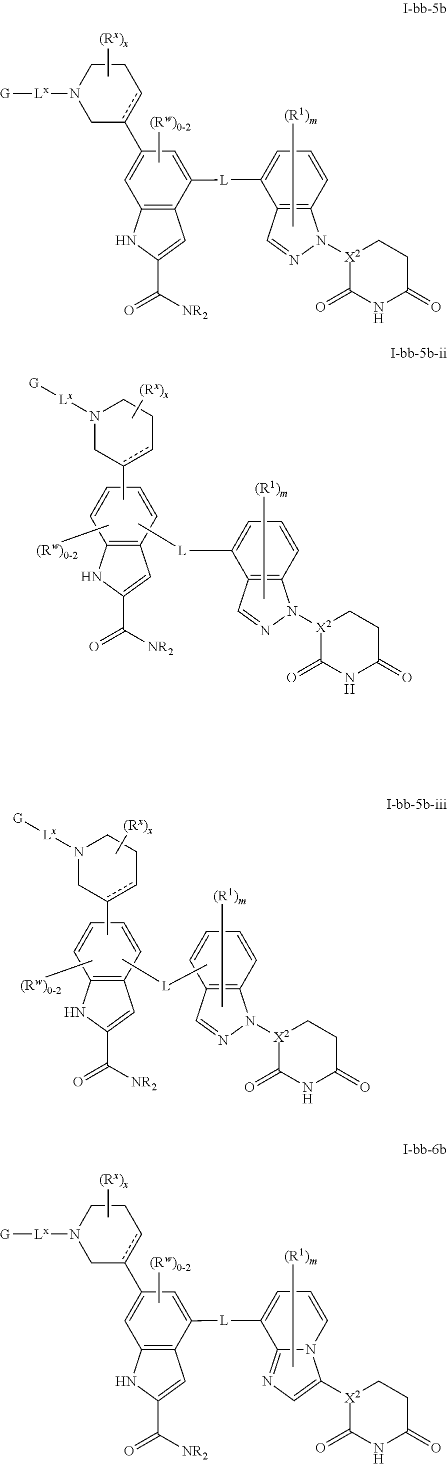

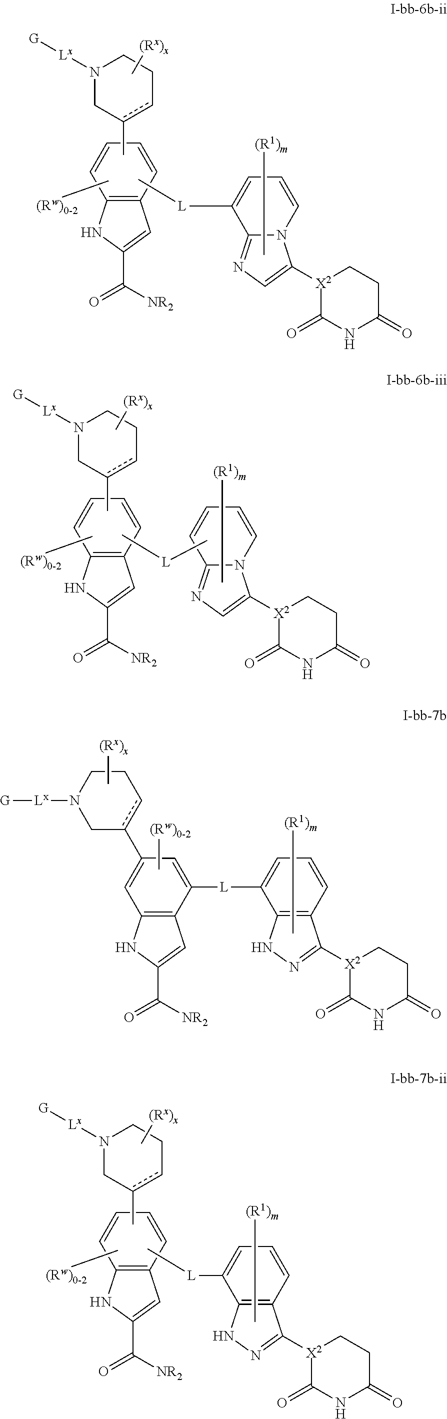

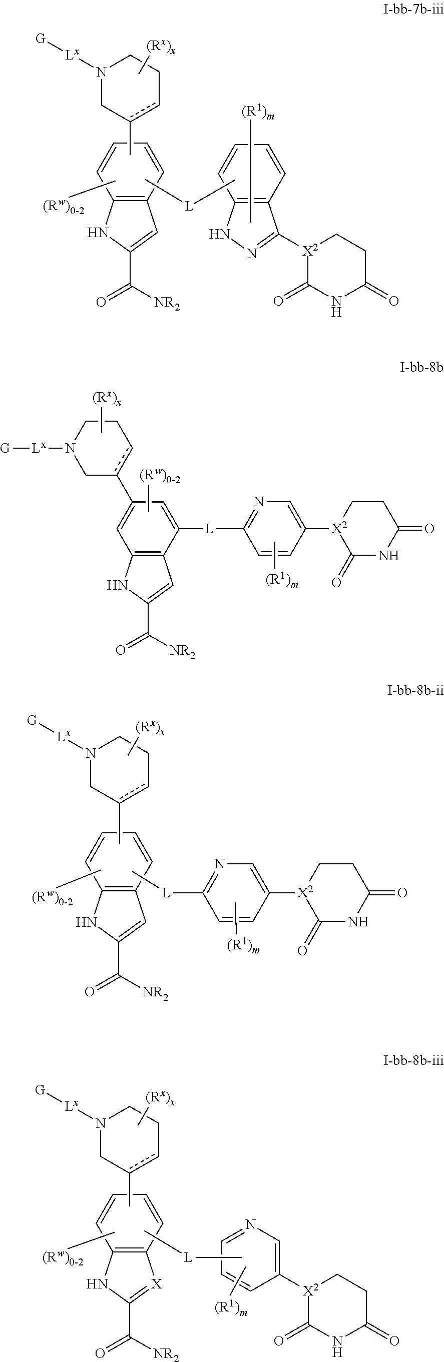

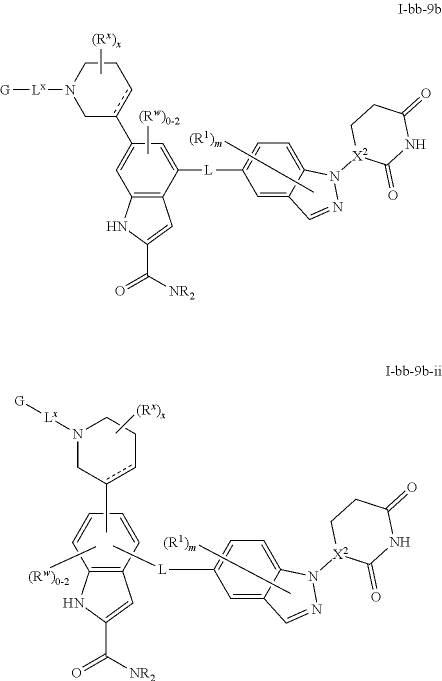

or a pharmaceutically acceptable salt thereof, wherein each variable is as defined above and described herein both individually and in combination, and wherein [symbol] represents a single or double bond.

In some embodiments, the present invention provides a compound of formula I-bb as a compound of any one of the following formulae:

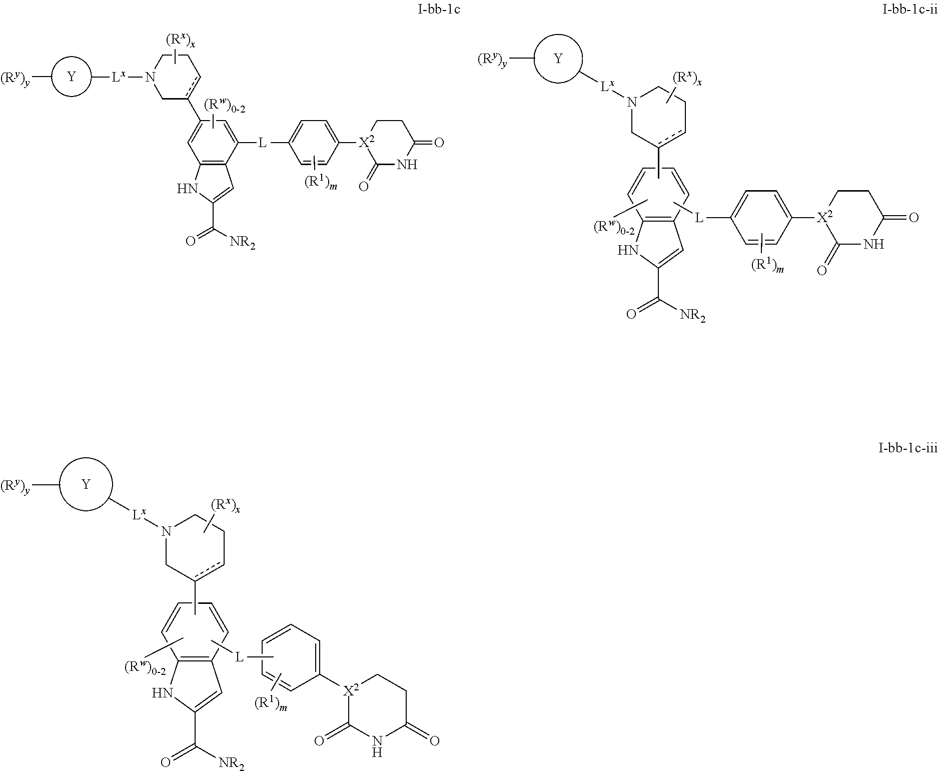

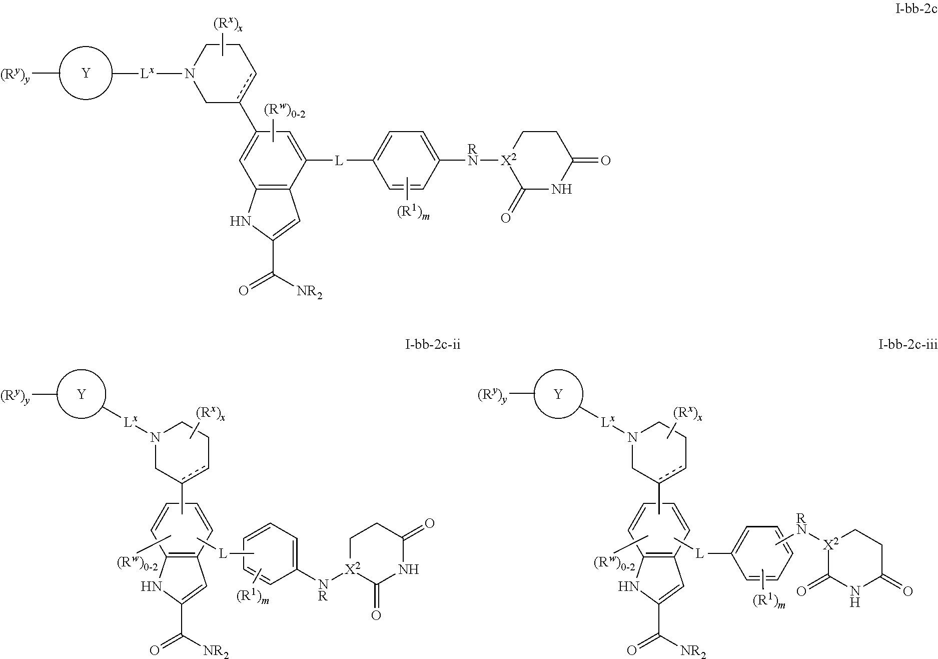

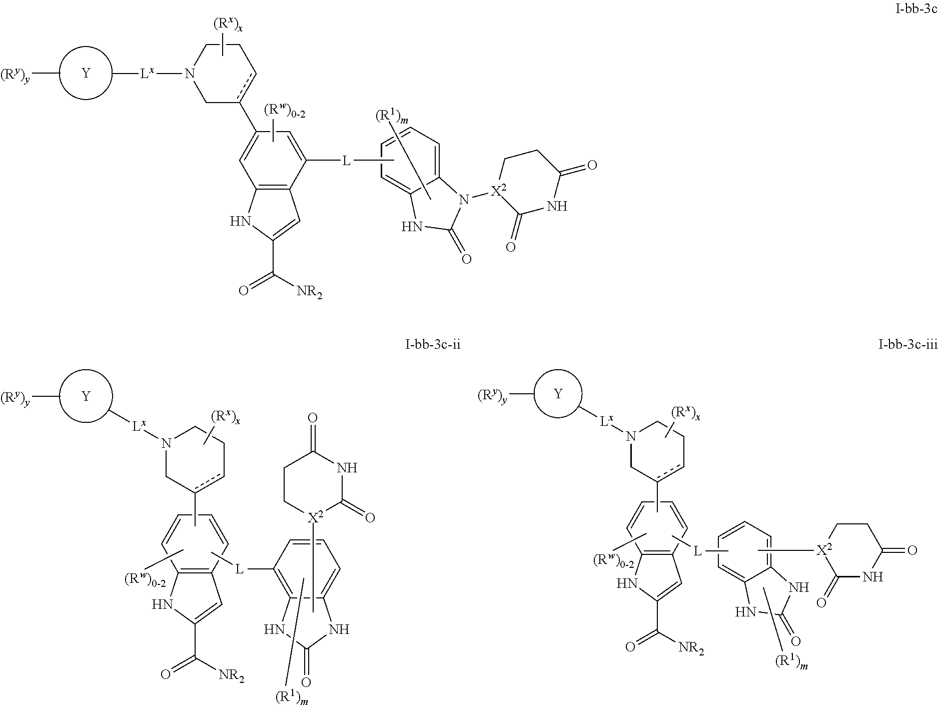

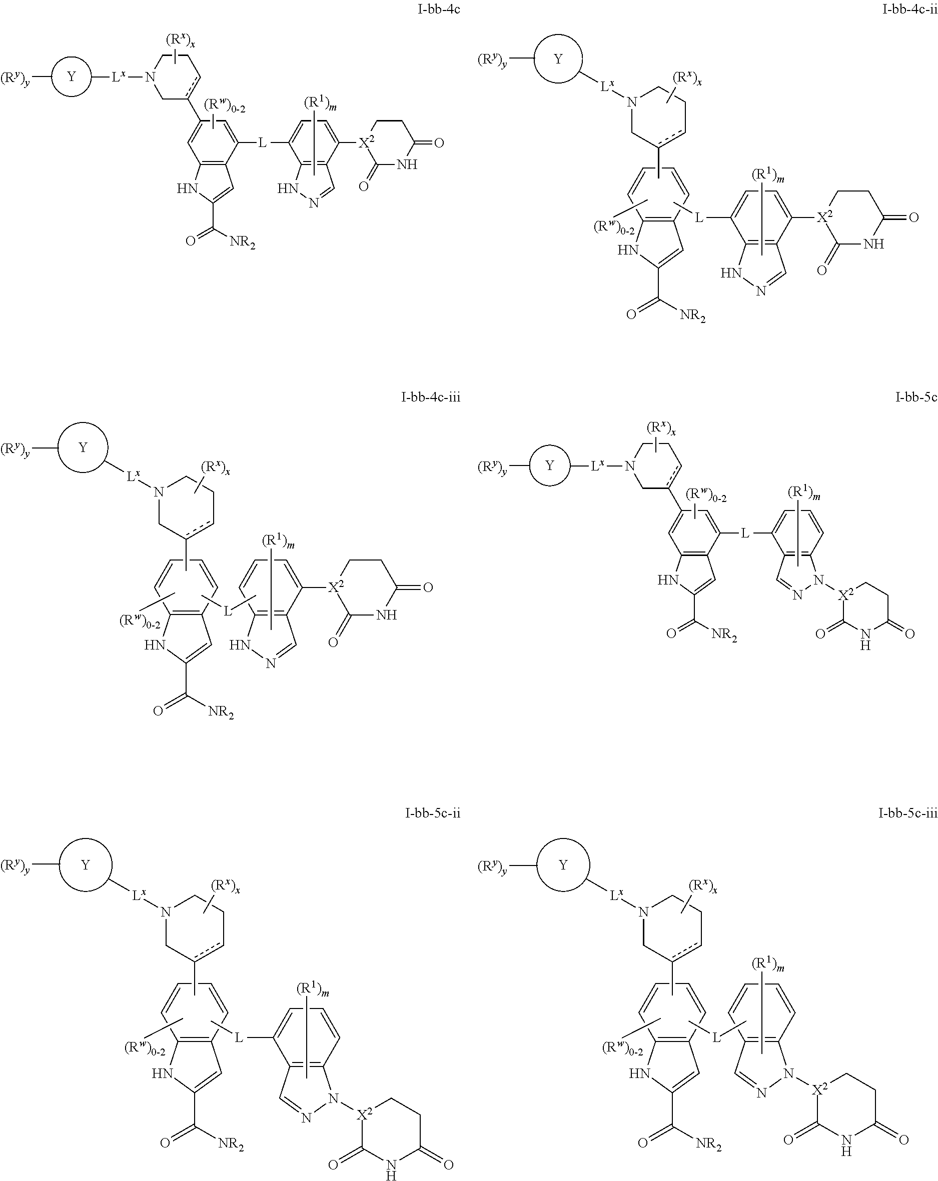

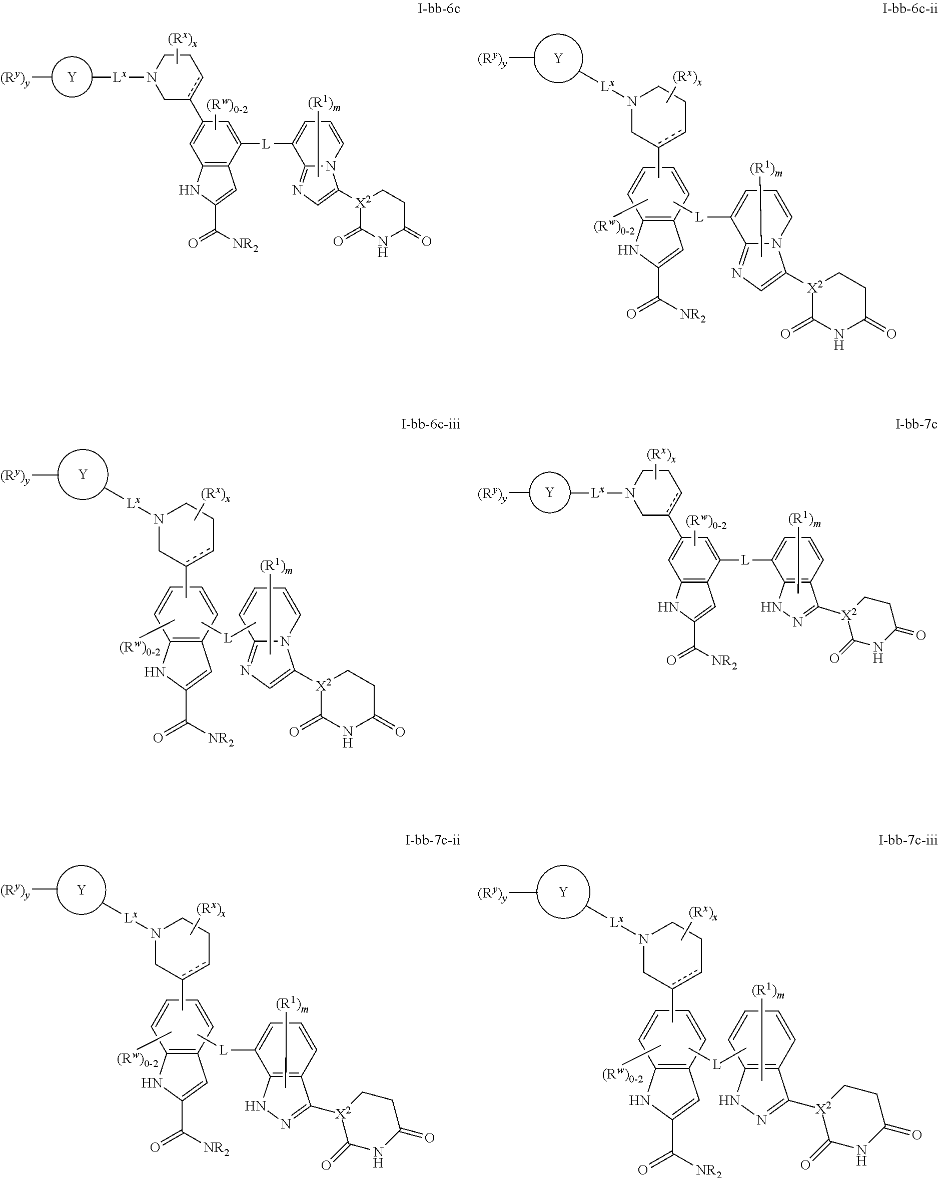

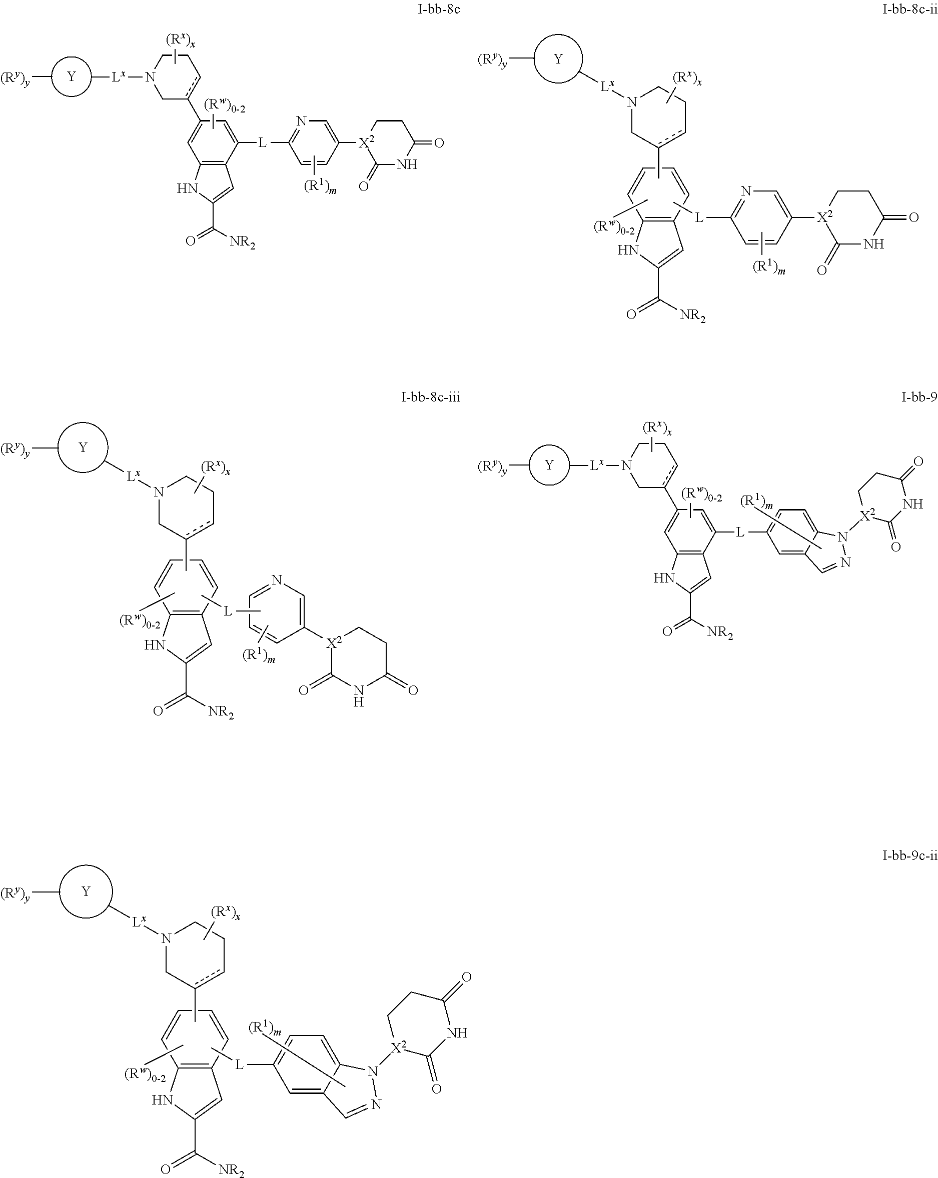

or a pharmaceutically acceptable salt thereof, wherein each variable is as defined above and described herein both individually and in combination.

In some embodiments, the present invention provides a compound of formula I-bb as a compound of any one of the following formulae:

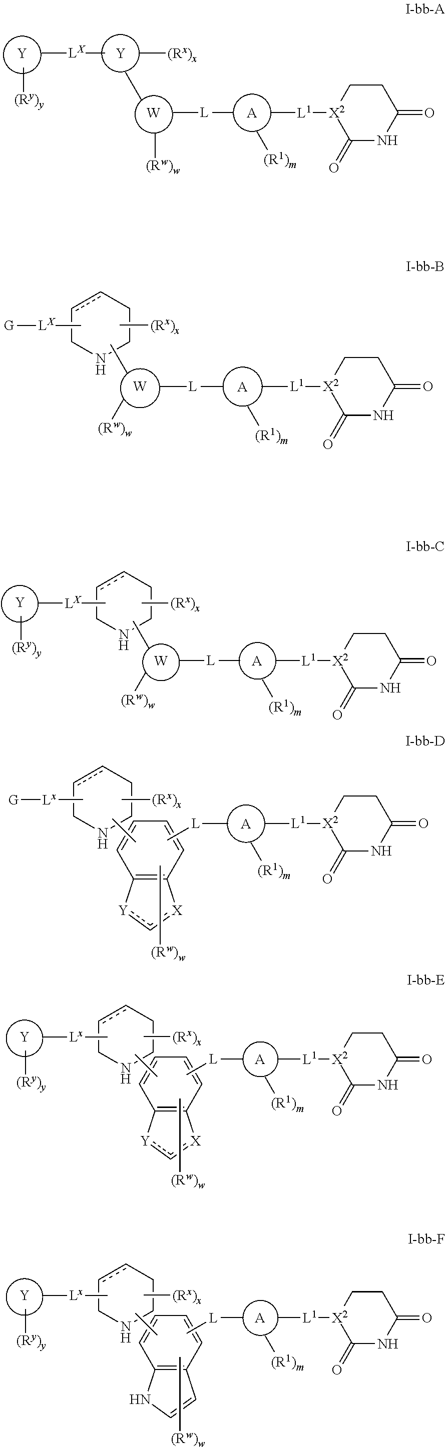

or a pharmaceutically acceptable salt thereof, wherein each variable is as defined above and described herein both individually and in combination;

In some embodiments, the present invention provides a compound of any one of the following formulae:

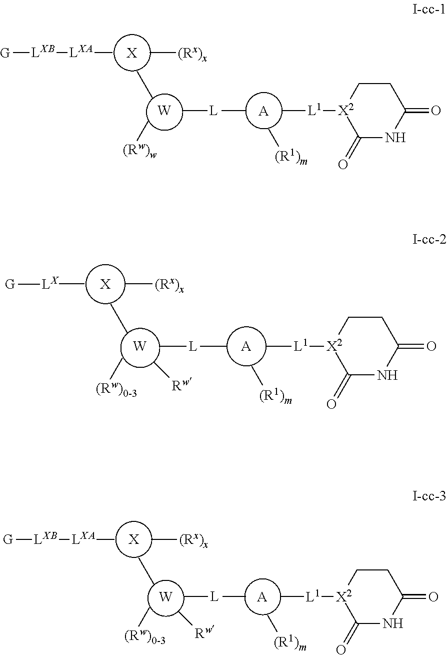

or a pharmaceutically acceptable salt thereof, wherein each Ring W, Ring X, R^{w}, R^{x}, w, x, G, L, L^{x}, Ring A, R¹, m, L¹, L^{XA}, L^{XB}, and X² is as defined above and described herein both individually and in combination, and wherein:
R^{w′} is R^{w}, wherein R^{w} as defined above and described herein.

In some embodiments, R^{w′} is —S(O)R, —S(O)₂R, —S(O)(NR)R, —S(O)₂NR₂, —C(O)R, —C(S)R, —C(NR)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, —NRS(O)₂R, or an optionally substituted group selected from C₁₋₆ aliphatic, phenyl, naphthalenyl, a 3-7 membered saturated or partially unsaturated carbocyclic or heterocyclic ring having 1-2 heteroatoms independently selected from nitrogen, oxygen, and sulfur, and a 5-10 membered monocyclic or bicyclic heteroaryl ring having 1-4 heteroatoms independently selected from nitrogen, oxygen, and sulfur.

In some embodiments, R^{w′} is —S(O)₂R. In some embodiments, R^{w′} is —S(O)₂NR₂. In some embodiments, R^{w′} is —S(O)(NR)R. In some embodiments, R^{w′} is —S(O)R. In some embodiments, R^{w′} is —C(O)R. In some embodiments, R^{w′} is —C(O)OR. In some embodiments, R^{w′} is —C(O)NR₂. In some embodiments, R^{w′} is —C(O)NROR. In some embodiments, R^{w′} is —OC(O)R. In some embodiments, R^{w′} is —OC(O)NR₂. In some embodiments, R^{w′} is —P(O)R₂. In some embodiments, R^{w′} is —P(O)(OR)₂. In some embodiments, R^{w′} is —OP(O)R₂. In some embodiments, R^{w′} is —OP(O)(OR)₂. In some embodiments, R^{w′} is —OP(O)(OR)NR₂. In some embodiments, R^{w′} is —OP(O)(NR₂)₂. In some embodiments. R^{w′} is —NRC(O)OR. In some embodiments, R^{w′} is —NRC(O)R. In some embodiments, R^{w′} is —NRC(O)N(R)₂. In some embodiments. R^{w′} is —NP(O)R₂. In some embodiments, R¹ is —NRP(O)(OR)₂. In some embodiments, R^{w} is —NRP(O)(OR)NR₂. In some embodiments, R^{w′} is —NRP(O)(NR₂)₂. In some embodiments, R^{w′} is —NRS(O)₂R.

In some embodiments, R^{w′}—C(O)OR. In some embodiments, R^{w′} is —C(O)NR₂. In some embodiments, R^{w′} is an optionally substituted phenyl. In some embodiments, R^{w′} is an optionally substituted 5-6 membered heteroaryl ring having 1-4 heteroatoms independently selected from nitrogen, oxygen, and sulfur.

In some embodiments, R^{w′} is —C(O)NHR. In some embodiments, R^{w′} is —C(O)NHR, wherein R of R^{w′} is optionally substituted C₁₋₆ aliphatic. In some embodiments, R^{w′} is —C(O)NHR, wherein R of R^{w′} is C₁₋₆ aliphatic, optionally substituted with —CN.

In some embodiments, R^{w′} is optionally substituted C₁₋₆ aliphatic. In some embodiments, R^{w′} is C₁₋₆ aliphatic, optionally substituted with —C(O)N(R^{∘})₂. In some embodiments. R^{w′} is C₁₋₆ aliphatic, optionally substituted with —NR^{∘}(O)N(R^{∘})₂. In some embodiments, R^{w′} is

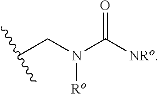

In some such embodiments, R^{∘} is hydrogen or C₁₋₆ aliphatic. In some embodiments, R^{w′} is C₁₋₆ aliphatic optionally substituted with —OR^{∘}, wherein R^{∘} is hydrogen or C₁₋₆ aliphatic. In some embodiments, R^{w′} is C₁₋₆ aliphatic optionally substituted with halogen (e.g., fluoro). In some embodiments, R^{w′} is —CH₂F, —CHF₂, or —CF₃.

In some embodiments, R^{w′} is an optionally substituted 3-7 membered saturated or partially unsaturated carbocyclic or heterocyclic ring having 1-2 heteroatoms independently selected from nitrogen, oxygen, and sulfur. In some embodiments, R^{w′} is an optionally substituted 5-6 membered saturated or partially unsaturated carbocyclic or heterocyclic ring having 1-2 heteroatoms independently selected from nitrogen, oxygen, and sulfur.

In some embodiments, R^{w′} is an optionally substituted

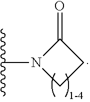

In some embodiments, R^{w′} is an optionally substituted

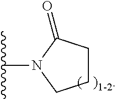

In some embodiments, R^{w′} is an optionally substituted

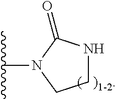

In some embodiments, R^{w′} is an optionally substituted 5-6 membered heteroaryl having 1-4 heteroatoms independently selected from nitrogen, oxygen, and sulfur. In some embodiments R^{w′} is an optionally substituted 5 membered heteroaryl having 1-4 heteroatoms independently selected from nitrogen, oxygen, and sulfur. In some embodiments, R^{w′} is an optionally substituted pyrazolyl, imidazolyl, triazolyl, or tetrazolyl. In some embodiments, R^{w′} is an optionally substituted imidazolyl, optionally substituted with —C(O)N(R^{∘})₂. In some embodiments. R^{w′} is an optionally substituted furanyl, thiophenyl, oxazolyl, isoxazolyl, isothiazolyl, thiazolyl, oxadiazolyl, or thiadiazolyl. In some embodiments, R^{w′} is furanyl, optionally substituted with —C(O)N(R^{∘})₂.

In some embodiments R^{w′} is an optionally substituted 6 membered heteroaryl having 1-4 heteroatoms independently selected from nitrogen, oxygen, and sulfur. In some embodiments R^{w′} is an optionally substituted 6 membered heteroaryl having 1-4 nitrogen heteroatoms. In some embodiments, R^{w′} is optionally substituted pyridinyl, pyrimidinyl, pyridazinyl, or triazinyl. In some embodiments. R^{w′} is an optionally substituted pyridinonyl, pyrazinonyl, or pyrimidinoyl.

In some embodiments, R^{w′} is —NHR, wherein R is an optionally substituted 5-6 membered heteroaryl having 1-4 heteroatoms independently selected from nitrogen, oxygen, and sulfur. In some embodiments, R^{w′} is —NHR, wherein R is an optionally substituted 6 membered heteroaryl having 1-4 heteroatoms independently selected from nitrogen, oxygen, and sulfur.

In embodiments, R^{w′} is —S(O)₂NHR, wherein R is an optionally substituted C₁₋₆ aliphatic. In embodiments, R^{w′} is —S(O)₂NHR, wherein R is an optionally substituted phenyl, 4-7 membered saturated or partially unsaturated carbocyclic or heterocyclic ring having 1-2 heteroatoms independently selected from nitrogen, oxygen, and sulfur, or a 5-6 membered heteroaryl ring having 1-4 heteroatoms independently selected from nitrogen, oxygen, and sulfur.

In embodiments, R^{w′} is —S(O)₂(NH)R. In some embodiments, R^{w′} is —S(O)₂(NH)R, wherein R is an optionally substituted C₁₋₆ aliphatic. In some embodiments, R^{w′} is —S(O)₂(NH)R, wherein R is an optionally substituted phenyl, 4-7 membered saturated or partially unsaturated carbocyclic or heterocyclic ring having 1-2 heteroatoms independently selected from nitrogen, oxygen, and sulfur, or a 5-6 membered heteroaryl ring having 1-4 heteroatoms independently selected from nitrogen, oxygen, and sulfur.

In some embodiments, R^{w′} is —C(S)R, —C(NR)R, —S(O)R, —S(O)₂R, —S(O)(NR)R, —S(O)₂NR₂, —S(O)R, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, or —NRS(O)₂R. In some embodiments, R^{w′} is —S(O)R, —S(O)₂R, —S(O)(NR)R, —S(O)₂NR₂, —S(O)R, —C(S)R, —C(NR)R, —C(O)R, —C(O)OR, —C(O)NR₂, —C(O)NROR, —OC(O)R, —OC(O)NR₂, —NRC(O)OR, —NRC(O)R, —NRC(O)N(R)₂, or —NRS(O)₂R. In some embodiments, R^{w′} is —S(O)R, —S(O)₂R, —S(O)(NR)R, —S(O)₂NR₂, —S(O)R, or —NRS(O)₂R. In some embodiments, R^{w′} is —C(O)R, —C(O)OR, —C(O)NR₂, —C(O)NROR, —OC(O)R, —OC(O)NR₂, —NRC(O)OR, —NRC(O)R, or —NRC(O)N(R)₂. In some embodiments, R^{w} is —S(O)R, —S(O)₂R, —S(O)(NR)R, —S(O)₂NR₂, —S(O)R, —C(S)R, —C(NR)R, —C(O)R, —C(O)OR, —C(O)NR₂, or —C(O)NROR. In some embodiments. R^{w′} is —C(S)R, —C(NR)R, —C(O)R, —C(O)OR, or —C(O)NR₂.

In some embodiments, R^{w′} is —C(O)R, wherein R is of R^{w′} is optionally substituted C₁e aliphatic. In some embodiments, R^{w′} is —C(O)R, wherein R is of R^{w′} is optionally substituted phenyl. In some embodiments, R^{w′} is —C(O)R, wherein R is of R^{w′} is an optionally substituted 4-7 membered saturated or partially unsaturated heterocyclic having 1-2 heteroatoms independently selected from nitrogen, oxygen, and sulfur. In some embodiments, R^{w′} is —C(O)R, wherein R is of R^{w′} is an optionally substituted 5-6 membered heteroaryl ring having 1-4 heteroatoms independently selected from nitrogen, oxygen, and sulfur.

In some embodiments, R^{w} is —C(S)R. In some embodiments, R^{w′} is —C(O)R, wherein R is of R^{w′} is optionally substituted C₁₋₆ aliphatic. In some embodiments, R^{w′} is —C(O)R, wherein R is of R^{w′} is optionally substituted phenyl. In some embodiments, R^{w′} is —C(O)R, wherein R is of R^{w′} is an optionally substituted 4-7 membered saturated or partially unsaturated heterocyclic having 1-2 heteroatoms independently selected from nitrogen, oxygen, and sulfur. In some embodiments, R^{w′} is —C(O)R, wherein R is of R^{w′} is an optionally substituted 5-6 membered heteroaryl ring having 1-4 heteroatoms independently selected from nitrogen, oxygen, and sulfur.

In some embodiments, R^{w′} is —C(O)OR, wherein R is of R^{w′} is optionally substituted C₁₋₆ aliphatic. In some embodiments, R^{w′} is —C(O)OR, wherein R is of R^{w′} is optionally substituted phenyl. In some embodiments, R^{w′} is —C(O)OR, wherein R is of R^{w′} is an optionally substituted 4-7 membered saturated or partially unsaturated heterocyclic having 1-2 heteroatoms independently selected from nitrogen, oxygen, and sulfur. In some embodiments, R^{w′} is —C(O)OR, wherein R is of R^{w′} is an optionally substituted 5-6 membered heteroaryl ring having 1-4 heteroatoms independently selected from nitrogen, oxygen, and sulfur.

In some embodiments, R^{w′} is —C(O)NR₂, wherein each R is of R^{w′} is independently hydrogen or an optionally substituted C₁₋₆ aliphatic. In some embodiments, R^{w′} is —C(O)NR₂, wherein each R is of R^{w′} is independently hydrogen or an optionally substituted phenyl. In some embodiments, R^{w′} is —C(O)NR₂, wherein each R is of R^{w′} is independently hydrogen or an optionally substituted 4-7 membered saturated or partially unsaturated heterocyclic having 1-2 heteroatoms independently selected from nitrogen, oxygen, and sulfur. In some embodiments, R^{w′} is —C(O)NR₂, wherein each R is of R^{w′} is independently hydrogen or an optionally substituted 5-6 membered heteroaryl ring having 1-4 heteroatoms independently selected from nitrogen, oxygen, and sulfur.

In some embodiments, R^{w′} is —C(NR)R. In some embodiments, R^{w′} is C(NR)R, wherein each R is of R^{w′} is independently hydrogen or an optionally substituted C₁₋₆ aliphatic. In some embodiments, R^{w′} is C(NR)R, wherein each R is of R^{w′} is independently hydrogen or an optionally substituted phenyl. In some embodiments, R^{w′} is C(NR)R, wherein each R is of R^{w′} is independently hydrogen or an optionally substituted 4-7 membered saturated or partially unsaturated heterocyclic having 1-2 heteroatoms independently selected from nitrogen, oxygen, and sulfur. In some embodiments, R^{w′} is C(NR)R, wherein each R is of R^{w′} is independently hydrogen or an optionally substituted 5-6 membered heteroaryl ring having 1-4 heteroatoms independently selected from nitrogen, oxygen, and sulfur.

In some embodiments, R^{w′} is —C(O)NHR, wherein R is an optionally substituted C₁₋₆ aliphatic. In some embodiments, R^{w′} is —C(O)NHR, wherein R is C₁₋₆ aliphatic. In some embodiments, R^{w′} is —C(O)NHR, wherein R is methyl, ethyl, or cyclopropyl. In some embodiments, R^{w′} is —C(O)NR₂, wherein the two R groups of R^{w′} are taken together with their intervening atoms to form a 3-10 membered saturated or partially unsaturated monocyclic, bicyclic, bridged bicyclic, or spirocyclic ring having 0-3 heteroatoms, in addition to the atom or adjacent atoms to which they are attached, independently selected form nitrogen, oxygen, and sulfur. In some embodiments, R^{w′} is —C(O)NR₂, wherein the two R groups of R^{w′} are taken together with their intervening atoms to form a 3-7 membered saturated or partially unsaturated monocyclic ring having 0-3 heteroatoms, in addition to the atom or adjacent atoms to which they are attached, independently selected form nitrogen, oxygen, and sulfur. In some embodiments, R^{w′} is —C(O)NR₂, wherein the two R groups of R^{w′} are taken together with their intervening atoms to form a 3-7 membered saturated or partially unsaturated monocyclic ring having 0 heteroatoms, in addition to the atom or adjacent atoms to which they are attached. In some embodiments, R^{w′} is —C(O)NR₂, wherein the two R groups of R^{w′} are taken together with their intervening atoms to form an aziridinyl, azetidinyl, diazetidinyl, pyrrolidinyl, or piperidinyl.

In some embodiments, R^{w′} is —C(O)NROR, wherein each R is of R^{w′} is independently hydrogen or an optionally substituted C₆ aliphatic. In some embodiments, R^{w′} is —C(O)NROR, wherein each R is of R^{w′} is independently hydrogen or an optionally substituted phenyl. In some embodiments, R^{w′} is —C(O)NROR, wherein each R is of R^{w′} is independently hydrogen or an optionally substituted 4-7 membered saturated or partially unsaturated heterocyclic having 1-2 heteroatoms independently selected from nitrogen, oxygen, and sulfur. In some embodiments, R^{w′} is —C(O)NROR, wherein each R is of R^{w′} is independently hydrogen or an optionally substituted 5-6 membered heteroaryl ring having 1-4 heteroatoms independently selected from nitrogen, oxygen, and sulfur.

In some embodiments, R^{w′} is —OC(O)R, wherein R is of R^{w′} is optionally substituted C₁₋₆ aliphatic. In some embodiments, R^{w′} is —OC(O)R, wherein R is of R^{w′} is optionally substituted phenyl. In some embodiments. R^{w′} is —OC(O)R, wherein R is of R^{w′} is an optionally substituted 4-7 membered saturated or partially unsaturated heterocyclic having 1-2 heteroatoms independently selected from nitrogen, oxygen, and sulfur. In some embodiments, R^{w′} is —OC(O)R, wherein R is of R^{w′} is an optionally substituted 5-6 membered heteroaryl ring having 1-4 heteroatoms independently selected from nitrogen, oxygen, and sulfur.

In some embodiments, R^{w′} is —OC(O)NR₂, wherein each R is of R^{w′} is independently hydrogen or an optionally substituted C₁₋₆ aliphatic. In some embodiments, R^{w′} is —OC(O)NR₂, wherein each R is of R^{w′} is independently hydrogen or an optionally substituted phenyl. In some embodiments. R^{w′} is —OC(O)NR₂, wherein each R is of R^{w′} is independently hydrogen or an optionally substituted 4-7 membered saturated or partially unsaturated heterocyclic having 1-2 heteroatoms independently selected from nitrogen, oxygen, and sulfur. In some embodiments, R^{w′} is —OC(O)NR₂, wherein each R is of R^{w′} is independently hydrogen or an optionally substituted 5-6 membered heteroaryl ring having 1-4 heteroatoms independently selected from nitrogen, oxygen, and sulfur.

In some embodiments, R^{w′} is —NRC(O)OR, wherein each R is of R^{w′} is independently hydrogen or an optionally substituted C₁₋₆ aliphatic. In some embodiments, R^{w′} is —NRC(O)OR, wherein each R is of R^{w′} is independently hydrogen or an optionally substituted phenyl. In some embodiments, R^{w′} is —NRC(O)OR, wherein each R is of R¹ is independently hydrogen or an optionally substituted 4-7 membered saturated or partially unsaturated heterocyclic having 1-2 heteroatoms independently selected from nitrogen, oxygen, and sulfur. In some embodiments, R^{w′} is —NRC(O)OR, wherein each R is of R^{w′} is independently hydrogen or an optionally substituted 5-6 membered heteroaryl ring having 1-4 heteroatoms independently selected from nitrogen, oxygen, and sulfur.

In some embodiments, R^{w′} is —NRC(O)R, wherein each R is of R^{w′} is independently hydrogen or an optionally substituted C₁₋₆ aliphatic. In some embodiments. R^{w′} is —NRC(O)R, wherein each R is of R^{w′} is independently hydrogen or an optionally substituted phenyl. In some embodiments, R^{w′} is —NRC(O)R, wherein each R is of R^{w′} is independently hydrogen or an optionally substituted 4-7 membered saturated or partially unsaturated heterocyclic having 1-2 heteroatoms independently selected from nitrogen, oxygen, and sulfur. In some embodiments, R^{w′} is —NRC(O)R, wherein each R is of R^{w′} is independently hydrogen or an optionally substituted 5-6 membered heteroaryl ring having 1-4 heteroatoms independently selected from nitrogen, oxygen, and sulfur.

In some embodiments, R^{w′} is —NRC(O)N(R)₂, wherein each R is of R^{w′} is independently hydrogen or an optionally substituted C₁₋₆ aliphatic. In some embodiments, R^{w′} is —NRC(O)N(R)₂, wherein each R is of R^{w′} is independently hydrogen or an optionally substituted phenyl. In some embodiments, R^{w′} is —NRC(O)N(R)₂, wherein each R is of R^{w′} is independently hydrogen or an optionally substituted 4-7 membered saturated or partially unsaturated heterocyclic having 1-2 heteroatoms independently selected from nitrogen, oxygen, and sulfur. In some embodiments, R^{w′} is —NRC(O)N(R)₂, wherein each R is of R^{w′} is independently hydrogen or an optionally substituted 5-6 membered heteroaryl ring having 1-4 heteroatoms independently selected from nitrogen, oxygen, and sulfur.

In some embodiments, R^{w′} is —NRS(O)₂R, wherein each R is of R^{w′} is independently hydrogen or an optionally substituted C₁₋₆ aliphatic. In some embodiments, R^{w′} is —NRS(O)₂R, wherein each R is of R^{w′} is independently hydrogen or an optionally substituted phenyl. In some embodiments, R^{w′} is —NRS(O)₂R, wherein each R is of R^{w′} is independently hydrogen or an optionally substituted 4-7 membered saturated or partially unsaturated heterocyclic having 1-2 heteroatoms independently selected from nitrogen, oxygen, and sulfur. In some embodiments, R^{w′} is —NRS(O)₂R, wherein each R is of R^{w′} is independently hydrogen or an optionally substituted 5-6 membered heteroaryl ring having 1-4 heteroatoms independently selected from nitrogen, oxygen, and sulfur.

In some embodiments, R^{w′} is —S(O)R, wherein R is of R^{w′} is optionally substituted C₁₋₆ aliphatic. In some embodiments, R^{w′} is —S(O)R, wherein R is of R^{w′} is optionally substituted phenyl. In some embodiments, R^{w′} is —S(O)R, wherein R is of R^{w′} is an optionally substituted 4-7 membered saturated or partially unsaturated heterocyclic having 1-2 heteroatoms independently selected from nitrogen, oxygen, and sulfur. In some embodiments, R^{w′} is —S(O)R, wherein R is of R^{w′} is an optionally substituted 5-6 membered heteroaryl ring having 1-4 heteroatoms independently selected from nitrogen, oxygen, and sulfur.

In some embodiments, R^{w′} is —S(O)₂R, wherein R is of R^{w′} is optionally substituted C₁₋₆ aliphatic. In some embodiments, R^{w′} is —S(O)₂R, wherein R is of R^{w′} is optionally substituted phenyl. In some embodiments, R^{w′} is —S(O)₂R, wherein R is of R^{w′} is an optionally substituted 4-7 membered saturated or partially unsaturated heterocyclic having 1-2 heteroatoms independently selected from nitrogen, oxygen, and sulfur. In some embodiments, R^{w′} is —S(O)₂R, wherein R is of R^{w′} is an optionally substituted 5-6 membered heteroaryl ring having 1-4 heteroatoms independently selected from nitrogen, oxygen, and sulfur.

In some embodiments, R^{w′} is —S(O)(NR)R, wherein each R is of R^{w′} is independently hydrogen or optionally substituted C₁₋₆ aliphatic. In some embodiments, R^{w′} is —S(O)(NR)R, wherein each R is of R^{w′} is independently hydrogen or optionally substituted phenyl. In some embodiments, R^{w′} is —S(O)(NR)R, wherein each R is of R^{w′} is independently hydrogen or an optionally substituted 4-7 membered saturated or partially unsaturated heterocyclic having 1-2 heteroatoms independently selected from nitrogen, oxygen, and sulfur. In some embodiments. R^{w′} is —S(O)(NR)R, wherein each R is of R^{w′} is independently hydrogen or an optionally substituted 5-6 membered heteroaryl ring having 1-4 heteroatoms independently selected from nitrogen, oxygen, and sulfur.

In some embodiments, R^{w′} is —S(O)₂NR₂, wherein each R is of R^{w′} is independently hydrogen or an optionally substituted C₁₋₆ aliphatic. In some embodiments, R^{w′} is —S(O)₂NR₂, wherein each R is of R^{w′} is independently hydrogen or an optionally substituted phenyl. In some embodiments, R¹ is —S(O)₂NR₂, wherein each R is of R^{w′} is independently hydrogen or an optionally substituted 4-7 membered saturated or partially unsaturated heterocyclic having 1-2 heteroatoms independently selected from nitrogen, oxygen, and sulfur. In some embodiments, R^{w′} is —S(O)₂NR₂, wherein each R is of R¹ is independently hydrogen or an optionally substituted 5-6 membered heteroaryl ring having 1-4 heteroatoms independently selected from nitrogen, oxygen, and sulfur.

In some embodiments, R^{w′} is an optionally substituted 3-7 membered saturated or partially unsaturated carbocyclic or heterocyclic ring having 1-2 heteroatoms independently selected from nitrogen, oxygen, and sulfur. In some embodiments, R^{w′} is an optionally substituted 3-7 membered saturated or partially unsaturated heterocyclic ring having 1-2 heteroatoms independently selected from nitrogen, oxygen, and sulfur, and is substituted with ═O. In some embodiments, R^{w′} is an optionally substituted 5-6 membered monocyclic heteroaryl ring having 1-4 heteroatoms independently selected from nitrogen, oxygen, and sulfur. In some embodiments, R^{w′} is an optionally substituted 5-6 membered monocyclic heteroaryl ring having 1-4 heteroatoms independently selected from nitrogen, oxygen, and sulfur, and is substituted with ═O. In some embodiments, R^{w′} is an optionally substituted:

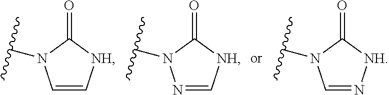

In some embodiments, R^{w′} is:

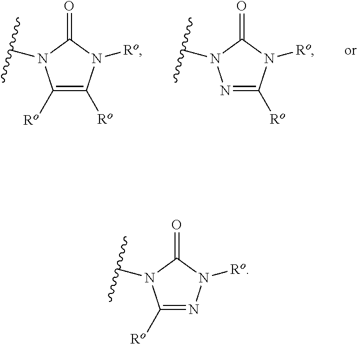

In some such embodiments, R^{∘} is hydrogen or C₁₋₆ aliphatic. In some embodiments, R^{w′} is:

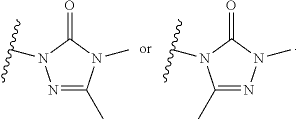

In some embodiments, R^{w′} is an optionally substituted:

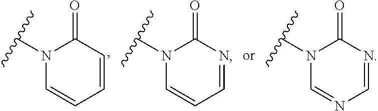

In some embodiments, R^{w′} is:

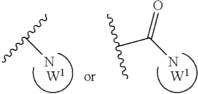

wherein Ring W¹ is as defined above and described herein.

In some embodiments, R^{w′} is:

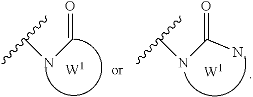

In some embodiments, R^{w′} is:

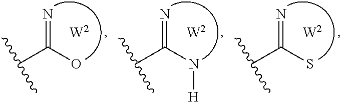

wherein Ring W² is as defined above and described herein.

In some embodiments, R^{w′} is —CO₂H, —C(O)NH₂, —C(O)NHMe, —C(O)NHEt, —C(O)NHnPr, —C(O)NHCH₂CH₂OH, —C(O)NMe₂, —C(O)N(Me)Et, —C(O)N(Me)nPr

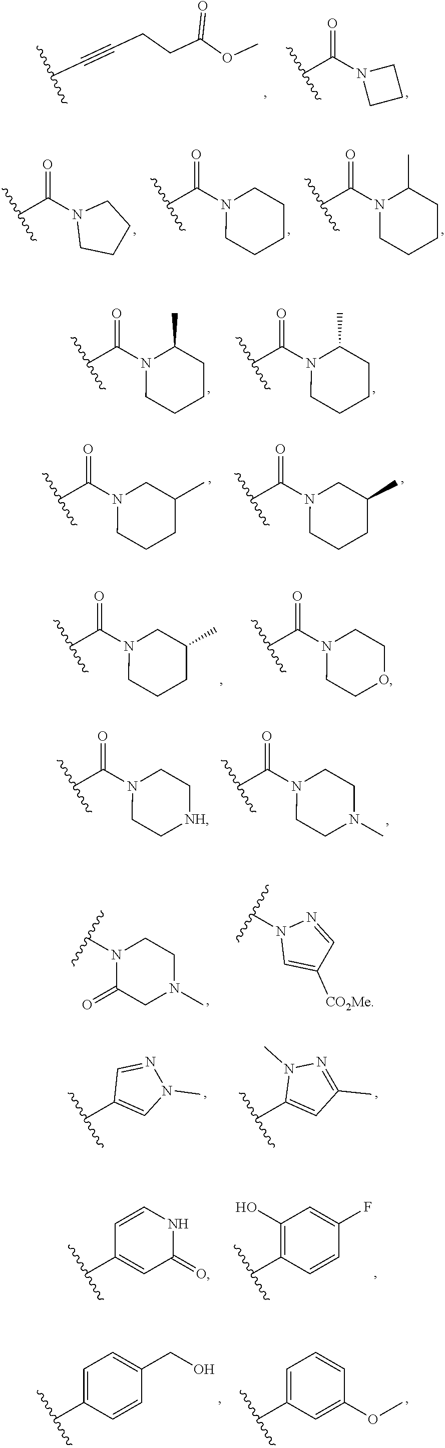

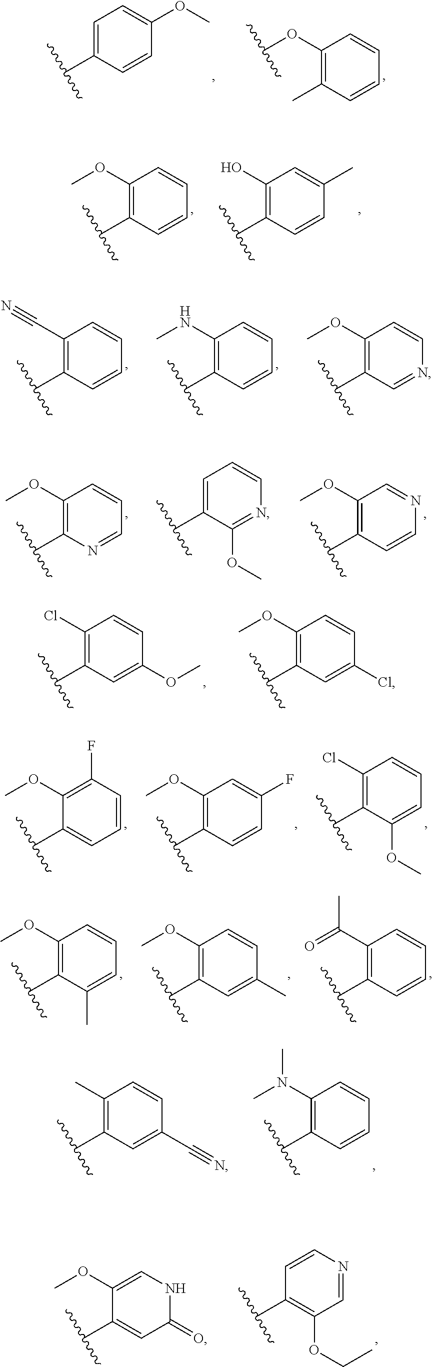

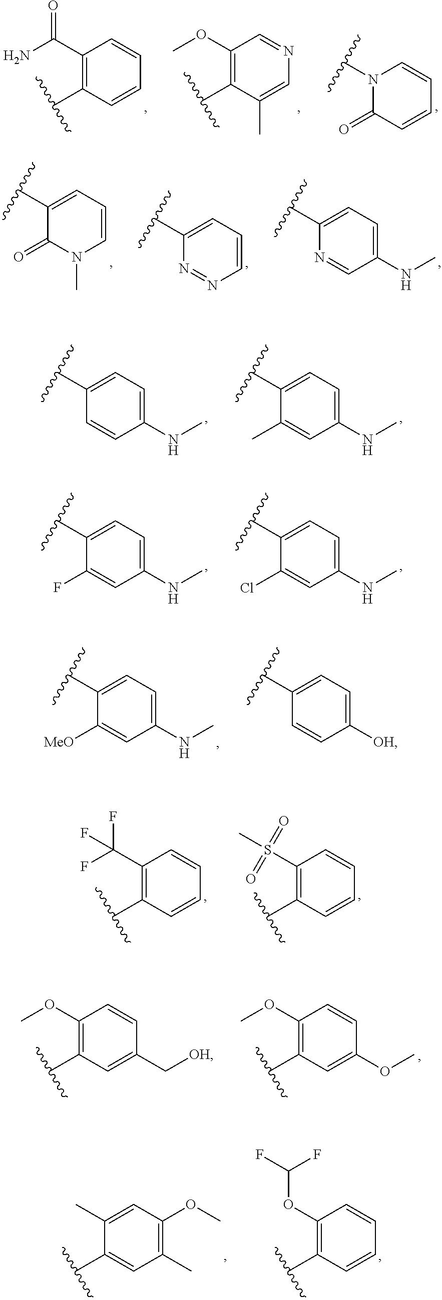

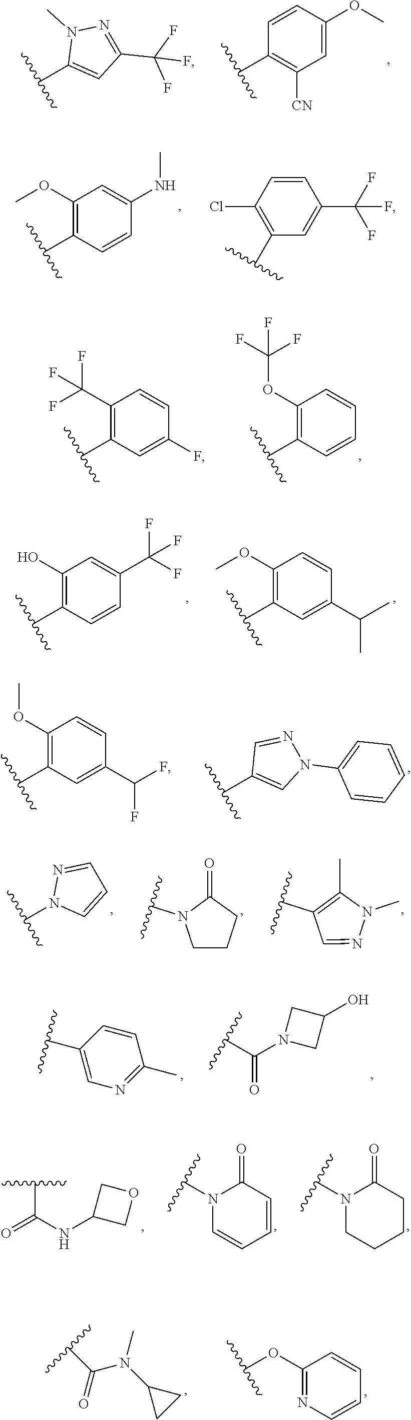

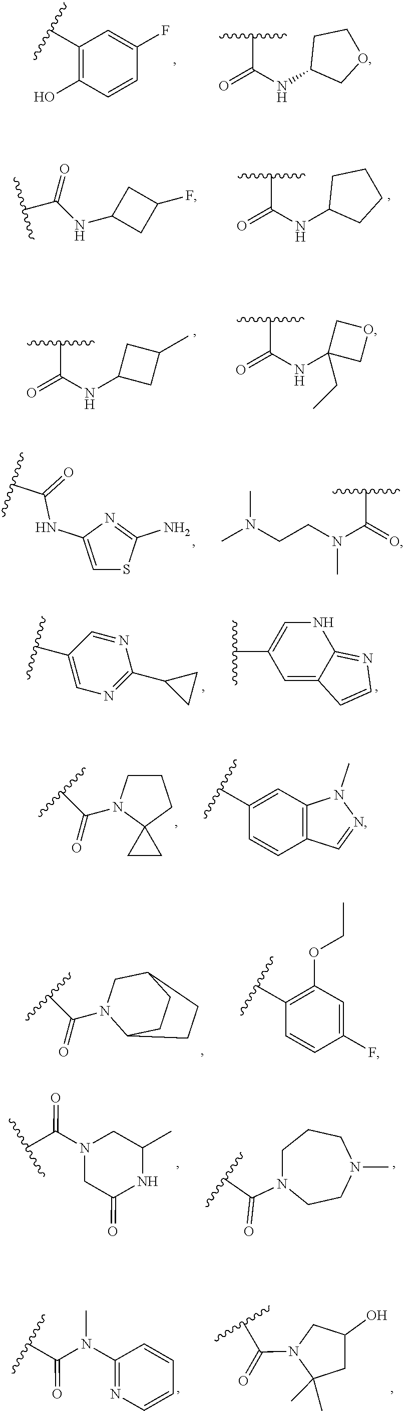

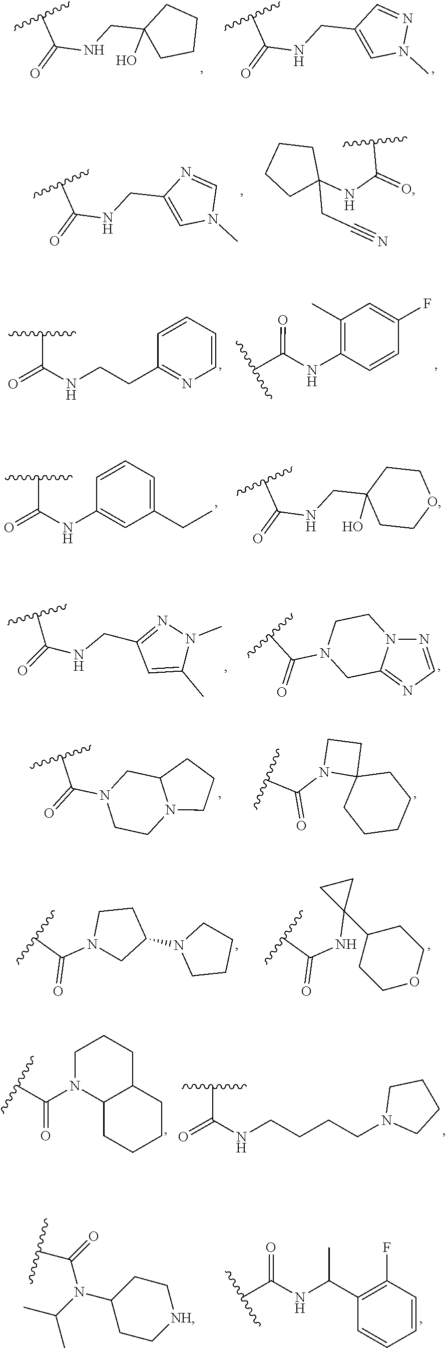

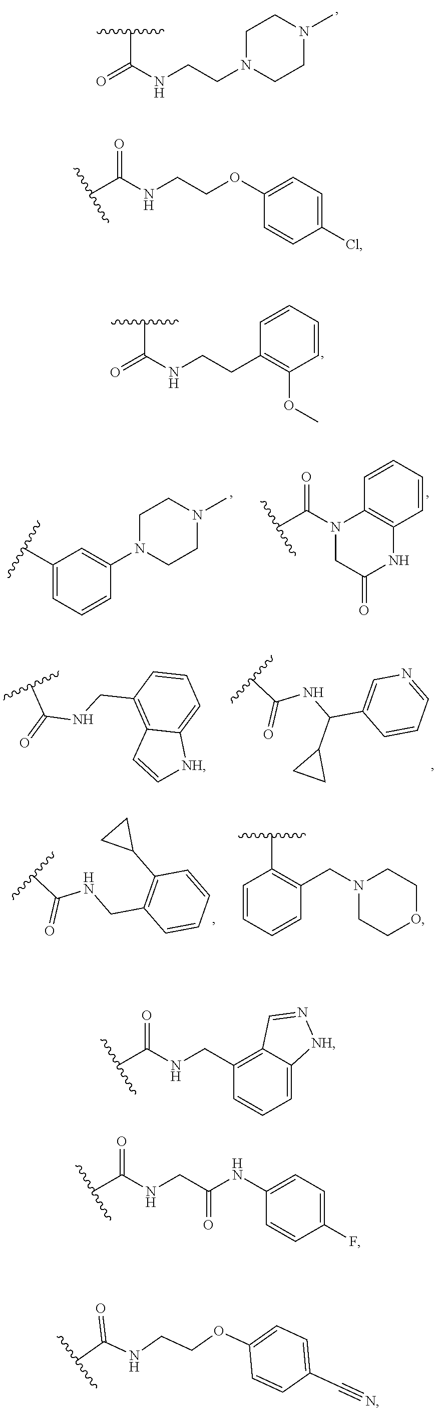

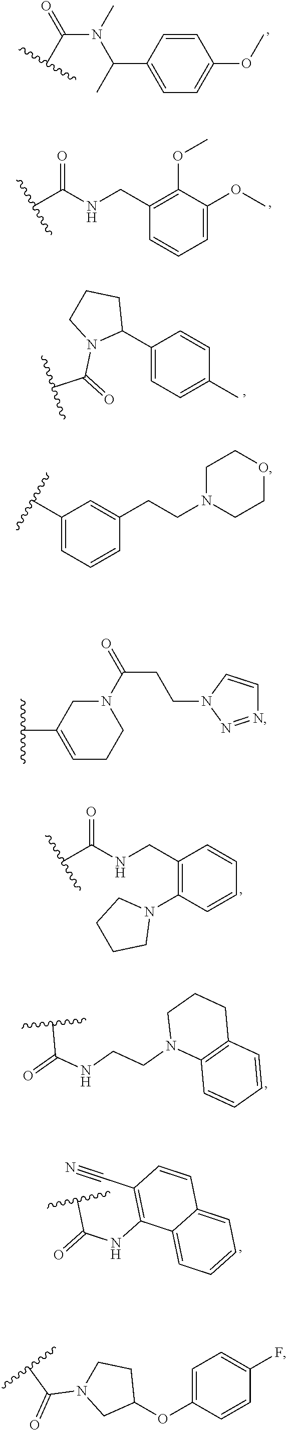

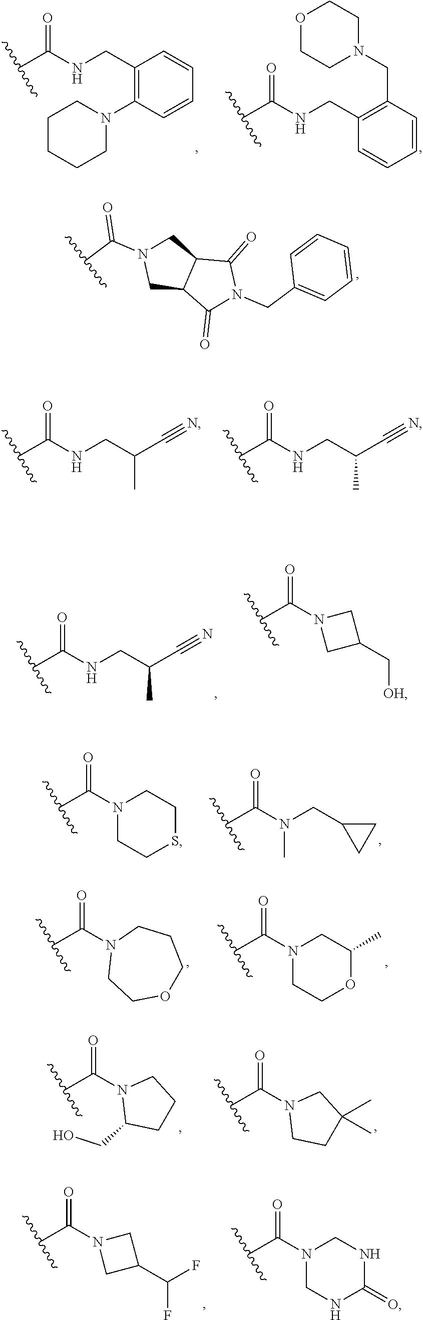

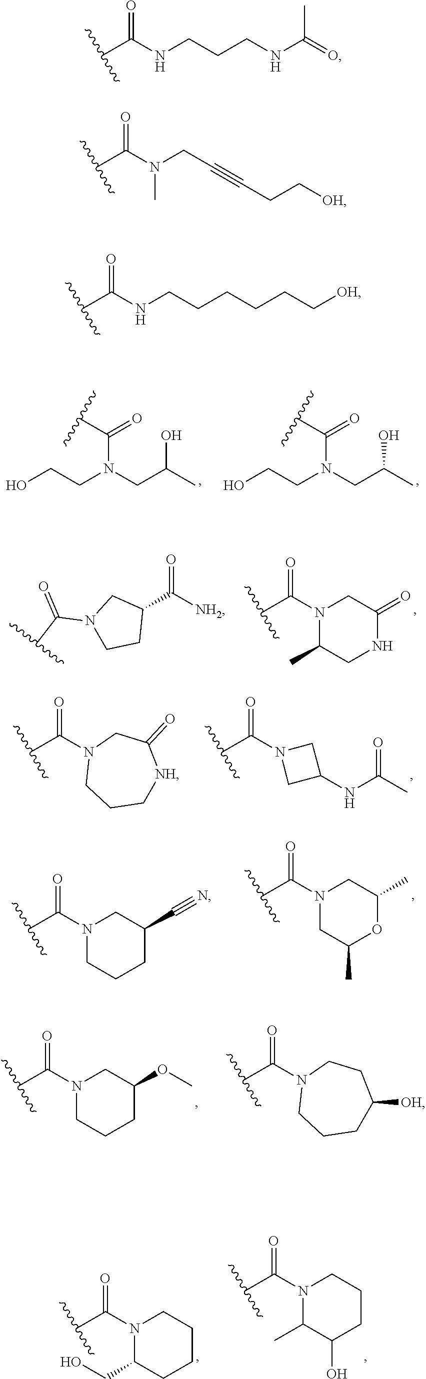

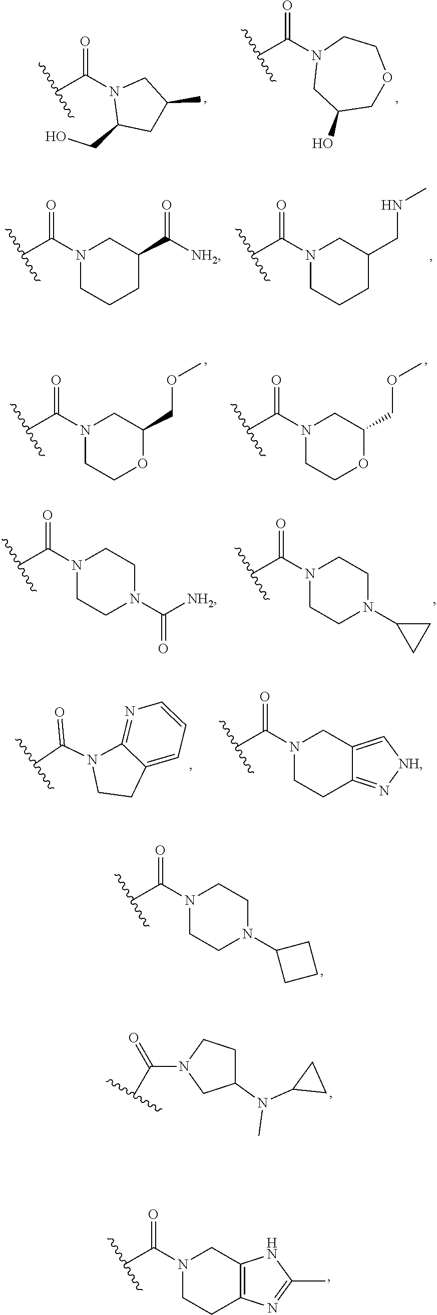

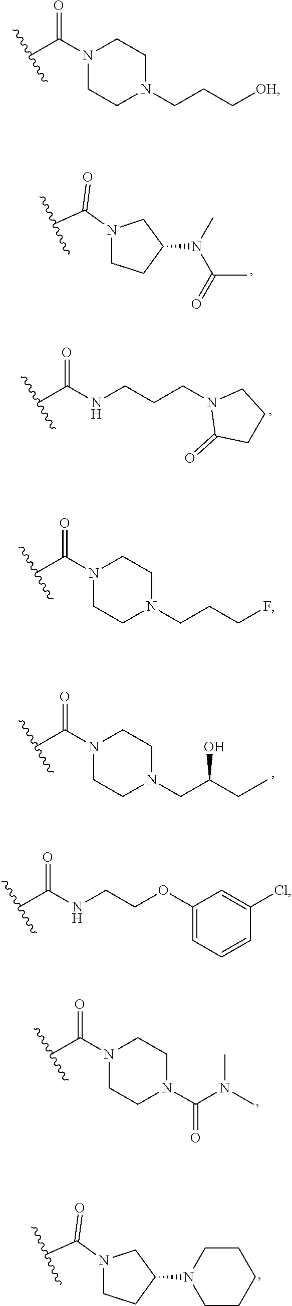

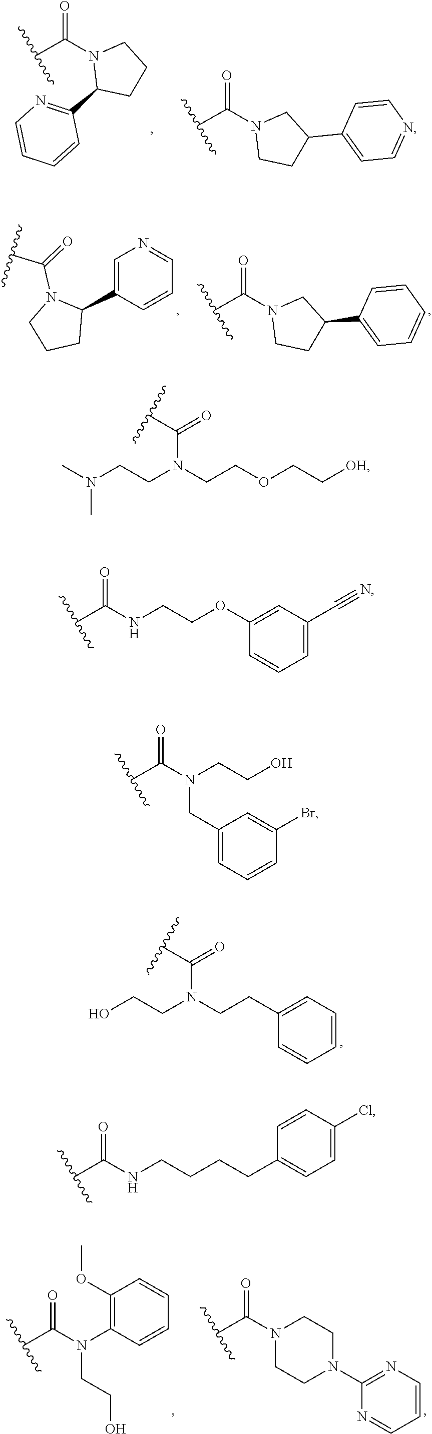

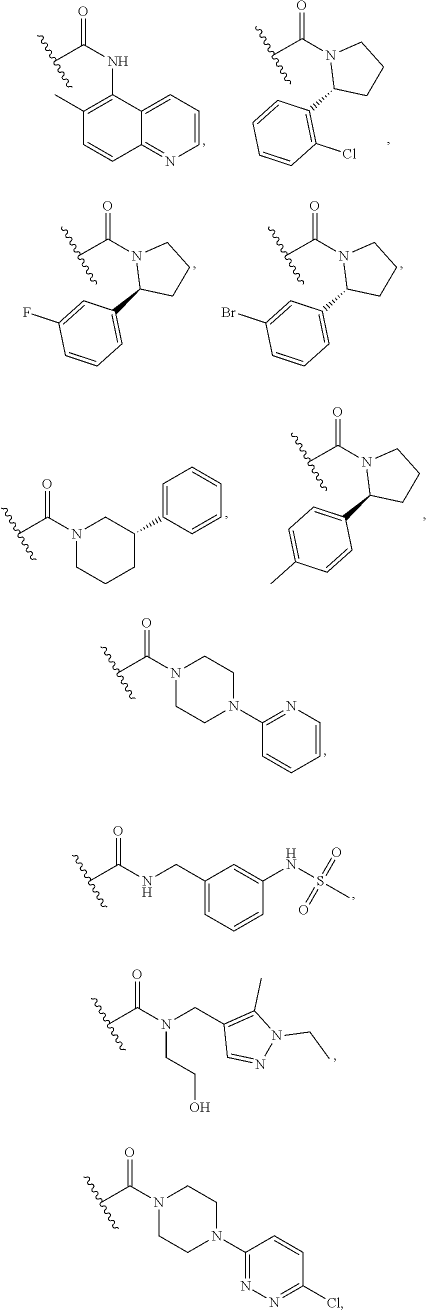

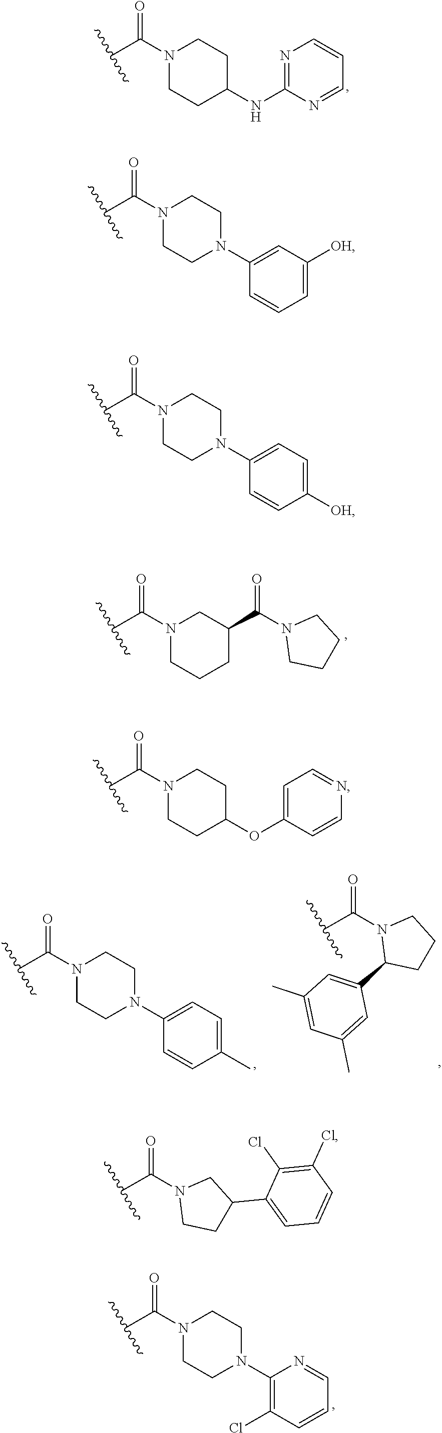

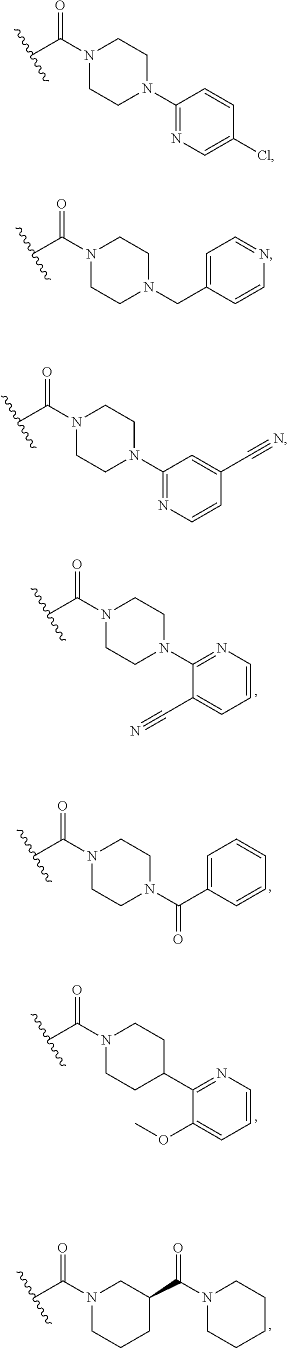

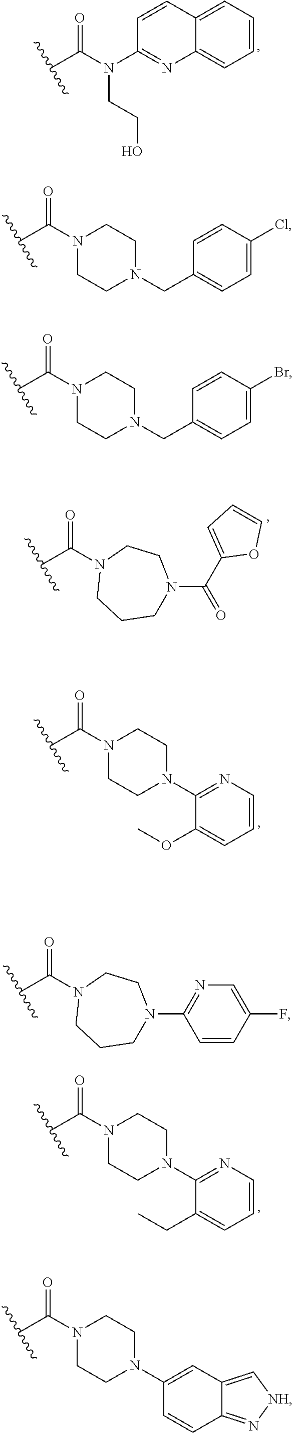

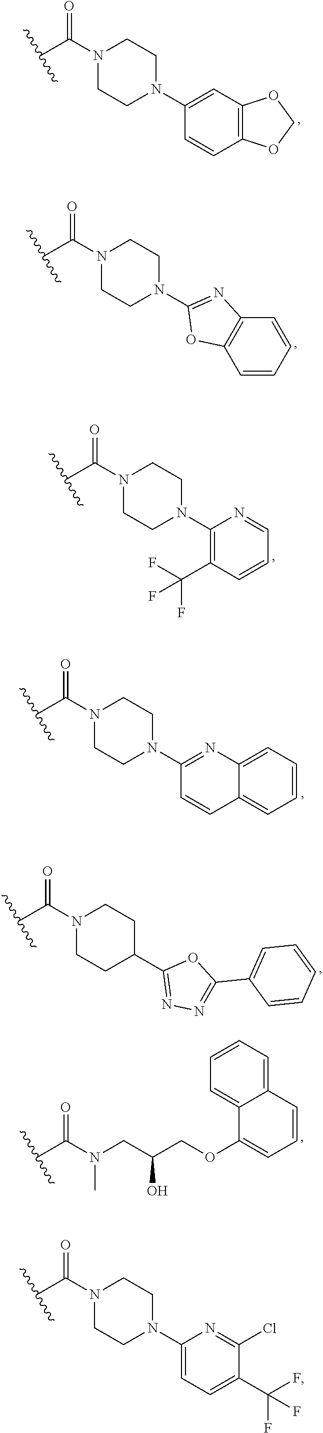

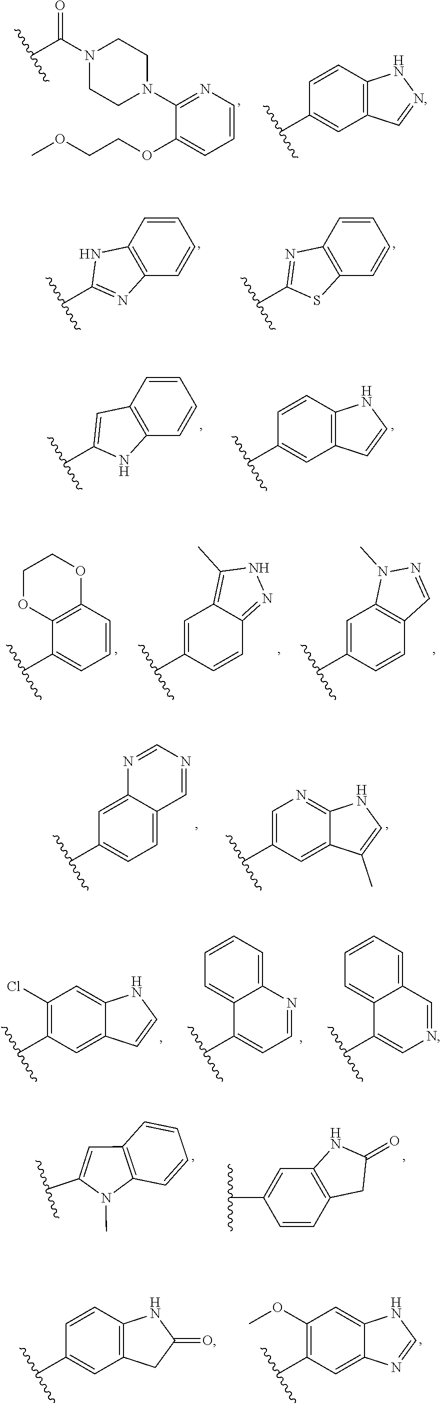

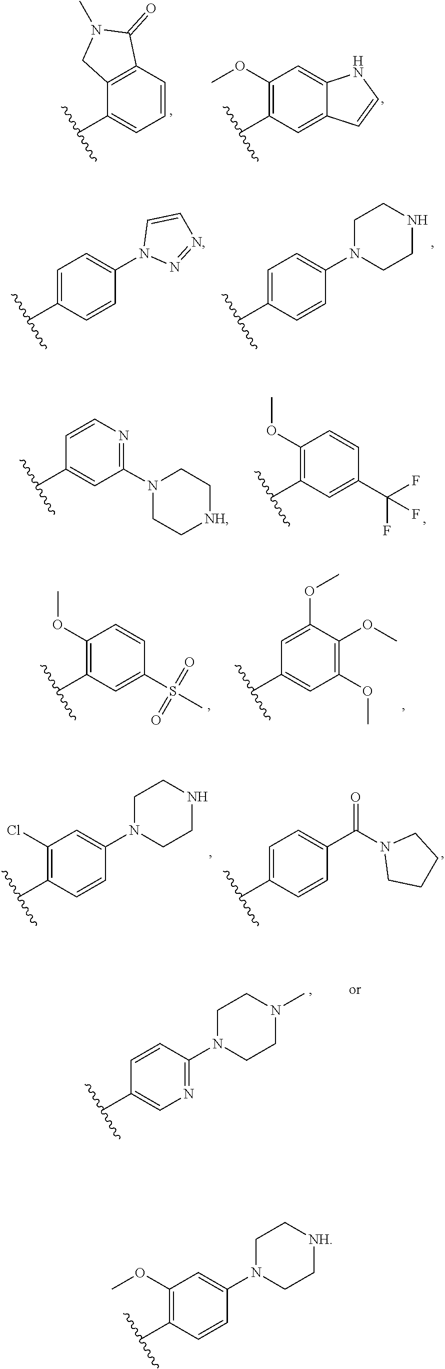

In some embodiments, R^{w′} is

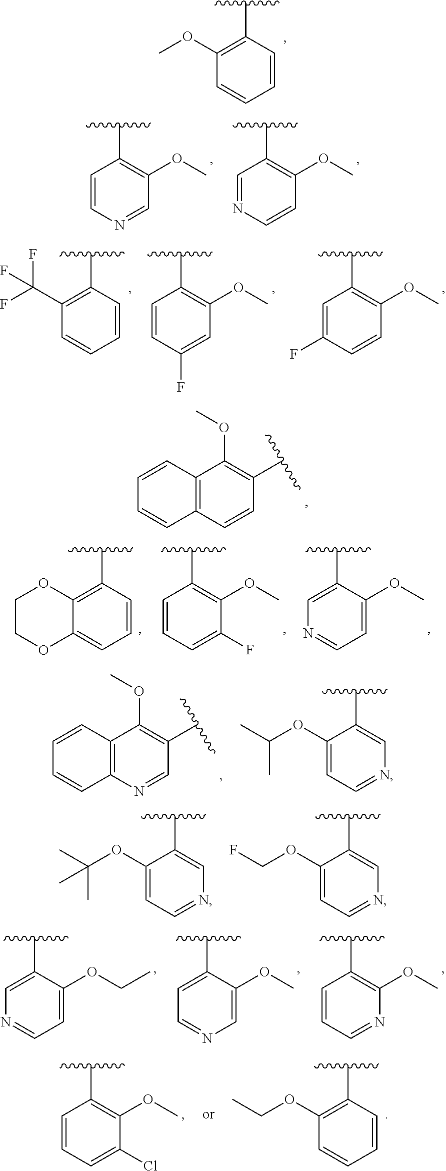

In some embodiments, R^{w′} is —S(O)₂NH₂, —S(O)₂N(CH₃)₂, —S(O)(NH)CH₃,

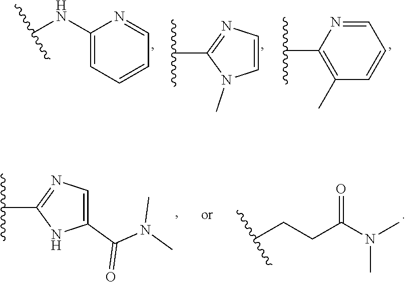

In some embodiments, the present invention provides a compound of any one of the following formulae:

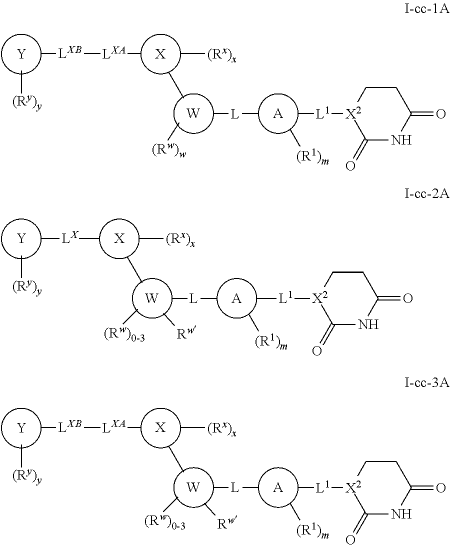

or a pharmaceutically acceptable salt thereof, wherein each variable is as defined above and described herein both individually and in combination.

In some embodiments, the present invention provides a compound of any one of the following formulae:

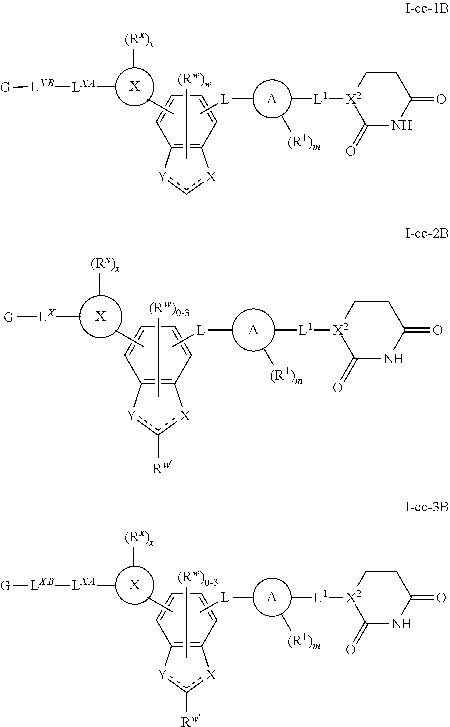

or a pharmaceutically acceptable salt thereof, wherein each variable is as defined above and described herein both individually and in combination.

In some embodiments, the present invention provides a compound of any one of the following formulae:

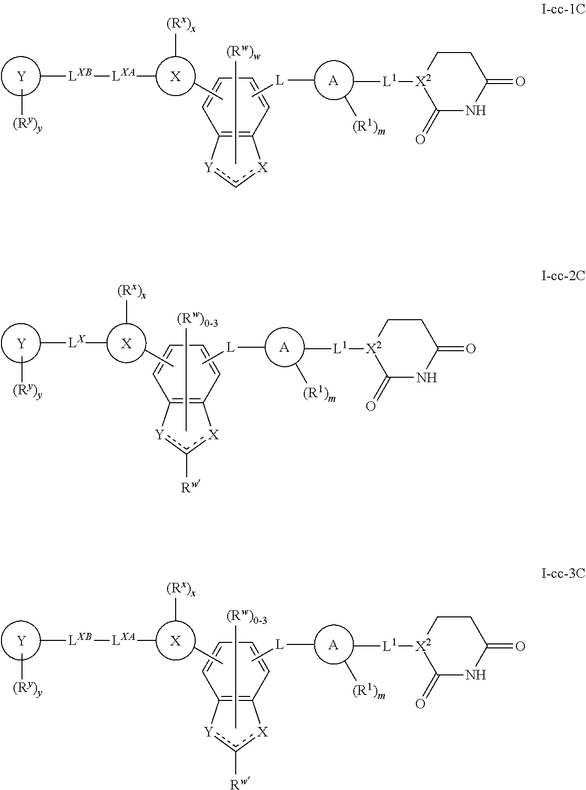

or a pharmaceutically acceptable salt thereof, wherein each variable is as defined above and described herein both individually and in combination.

In some embodiments, the present invention provides a compound of any one of the following formulae:

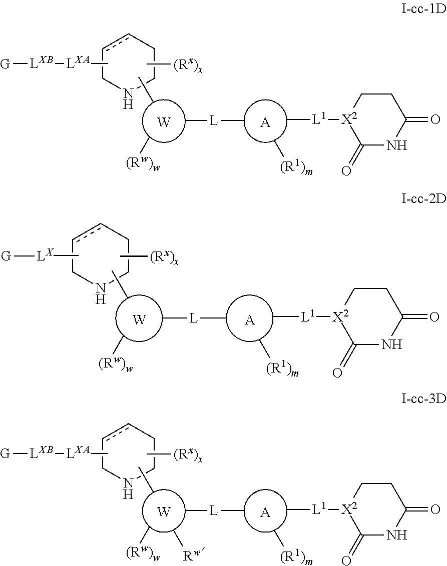

or a pharmaceutically acceptable salt thereof, wherein each variable is as defined above and described herein both individually and in combination.

In some embodiments, the present invention provides a compound of any one of the following formulae:

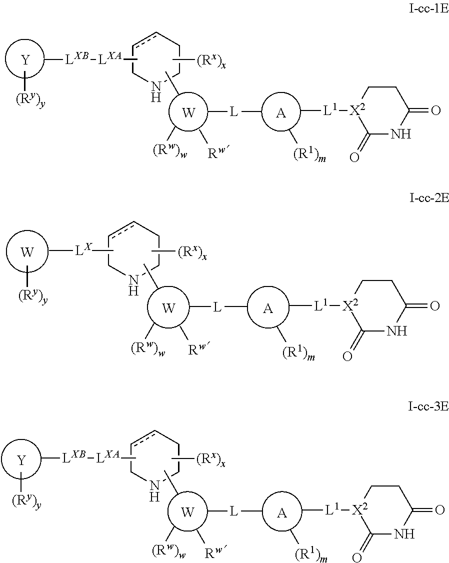

or a pharmaceutically acceptable salt thereof, wherein each variable is as defined above and described herein both individually and in combination.

In some embodiments, the present invention provides a compound of any one of the following formulae:

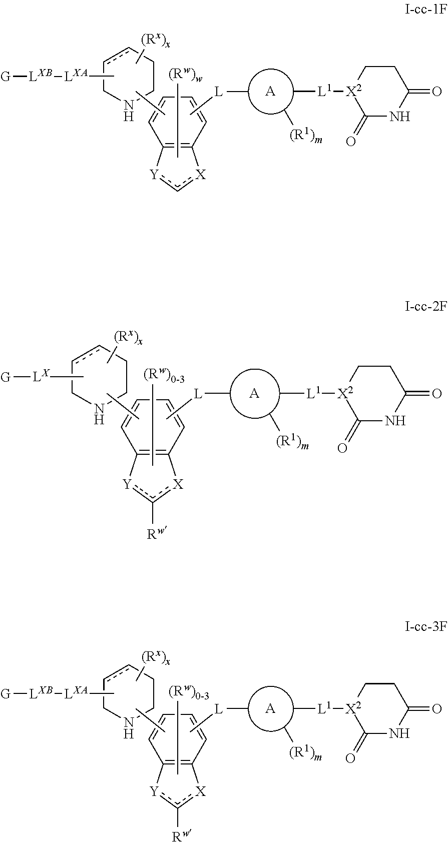

or a pharmaceutically acceptable salt thereof, wherein each variable is as defined above and described herein both individually and in combination.

In some embodiments, the present invention provides a compound of any one of the following formulae:

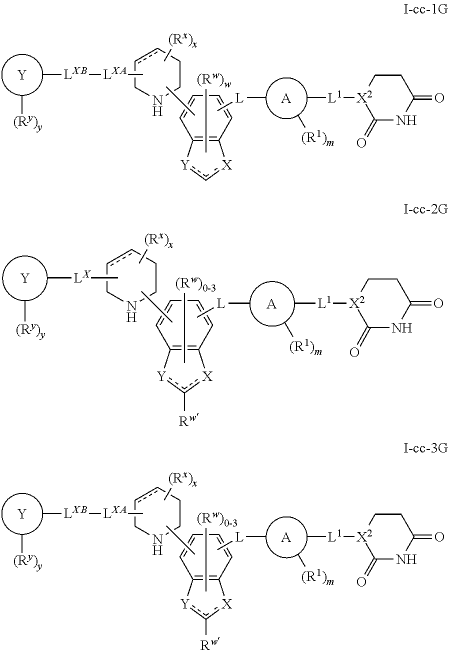

or a pharmaceutically acceptable salt thereof, wherein each variable is as defined above and described herein both individually and in combination.

In some embodiments, the present invention provides a compound of any one of the following formulae:

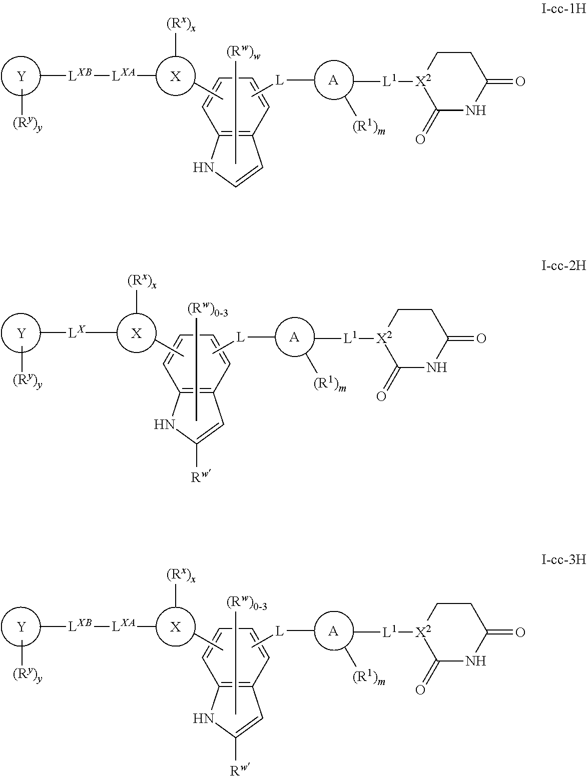

or a pharmaceutically acceptable salt thereof, wherein each variable is as defined above and described herein both individually and in combination.

In some embodiments, the present invention provides a compound of any one of the following formulae:

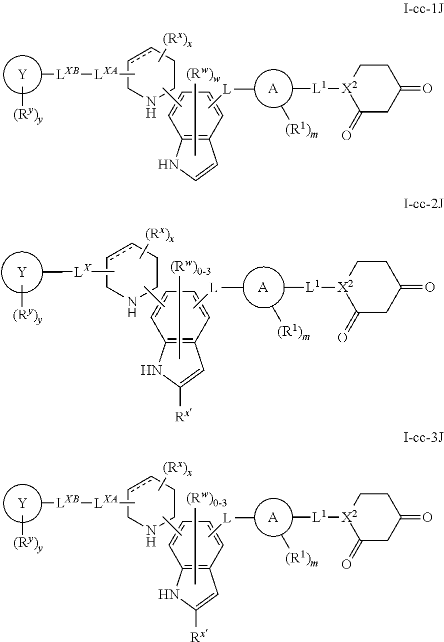

or a pharmaceutically acceptable salt thereof, wherein each variable is as defined above and described herein both individually and in combination.

In some embodiments of any one of formulae I-cc-1, I-cc-2, I-cc-3, I-cc-1A, I-cc-2A, I-cc-3A, I-cc-1B, I-cc-2B, I-cc-3B, I-cc-1C, I-cc-2C, I-cc-3C, I-cc-1D, I-cc-2D, I-cc-3D, I-cc-1E, I-cc-2E, I-cc-3E, I-cc-1F, I-cc-2F, I-cc-3F, I-cc-1G, I-cc-2G, I-cc-3G, I-cc-1H, I-cc-2H, I-cc-3H. I-cc-1J, I-cc-2J, or I-cc-3J, the structure

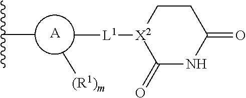

is of any one of formula I-aa-10′, I-aa-11′, I-aa-12′, I-aa-13′, or I-aa-14′:

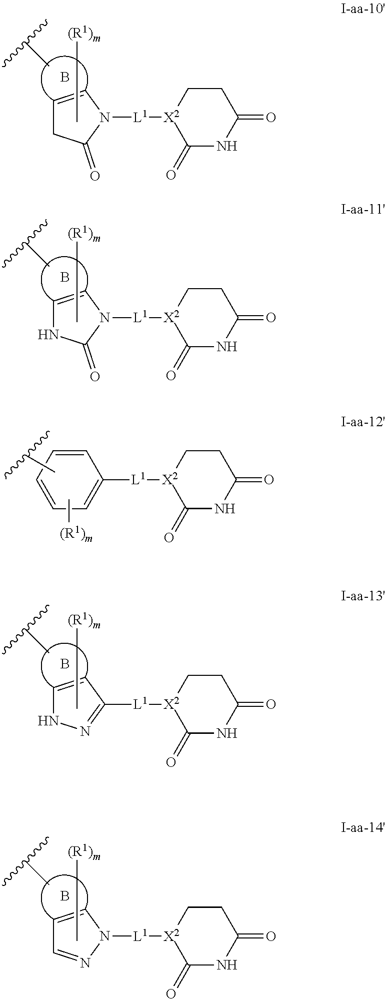

or a pharmaceutically acceptable salt thereof, wherein each variable is as defined above and described herein both individually and in combination.

In some embodiments of any one of formulae I-cc-1, I-cc-2, I-cc-3, I-cc-1A, I-cc-2A, I-cc-3A, I-cc-1B, I-cc-2B, I-cc-3B, I-cc-1C, I-cc-2C. I-cc-3C, I-cc-1D. I-cc-2D, I-cc-3D, I-cc-IE, I-cc-2E, I-cc-3E, I-cc-IF. I-cc-2F, I-cc-3F. I-cc-1G, I-cc-2G, I-cc-3G, I-cc-1H, I-cc-2H, I-cc-3H, I-cc-IJ, I-cc-2J, or I-cc-3J, the structure

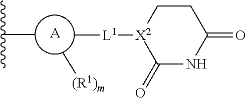

is of any one of formula I-aa-10a′, I-aa-11a′, I-aa-12a′, I-aa-12b′, I-aa-13a′, or I-aa-14a′:

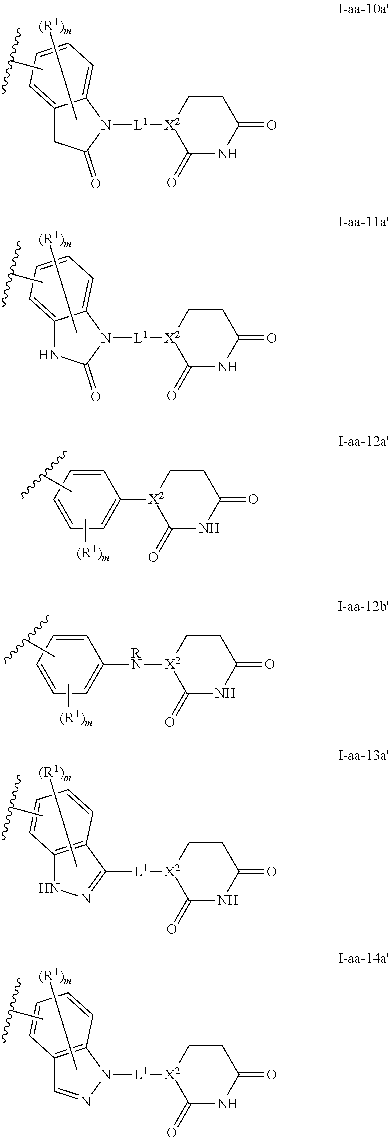

or a pharmaceutically acceptable salt thereof, wherein each variable is as defined above and described herein both individually and in combination.

In some embodiments, the present invention provides a compound of any one of the following formulae:

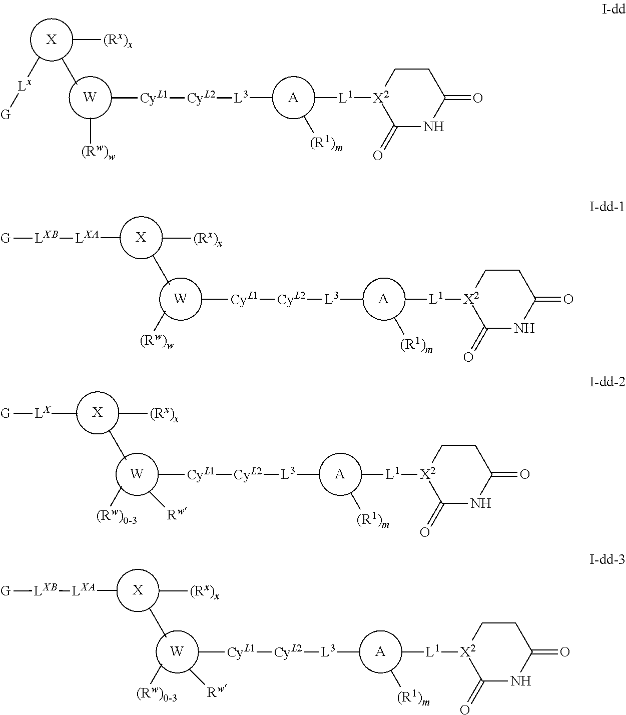

or a pharmaceutically acceptable salt thereof, wherein each variable is as defined above and described herein both individually and in combination.

In some embodiments, the present invention provides a compound of any one of the following formulae:

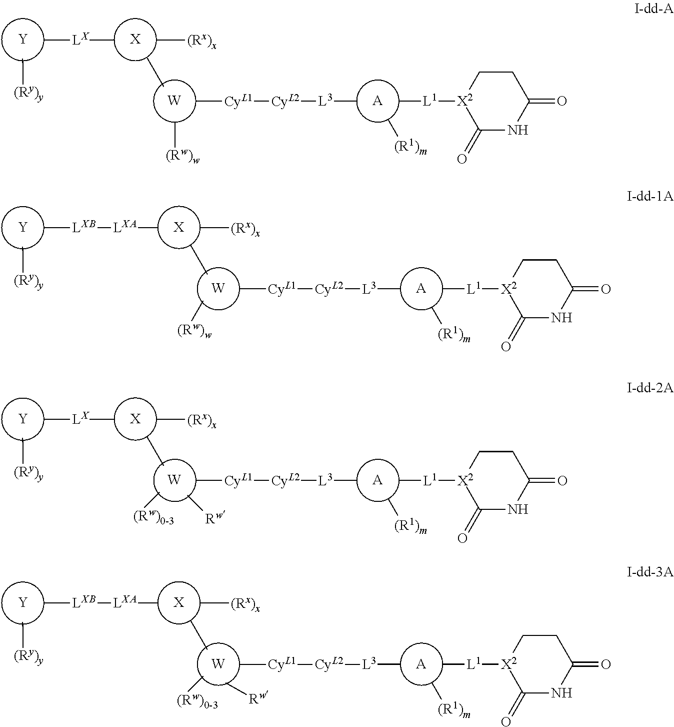

or a pharmaceutically acceptable salt thereof, wherein each variable is as defined above and described herein both individually and in combination.

In some embodiments, the present invention provides a compound of any one of the following formulae:

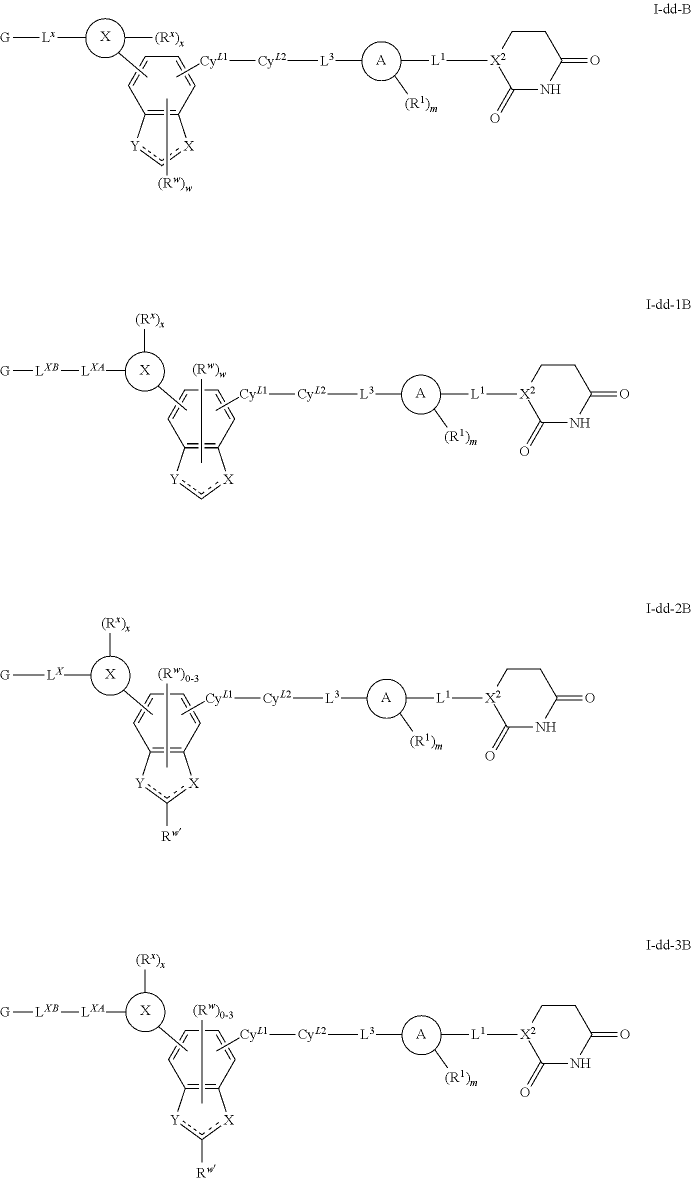

or a pharmaceutically acceptable salt thereof, wherein each variable is as defined above and described herein both individually and in combination.

In some embodiments, the present invention provides a compound of any one of the following formulae:

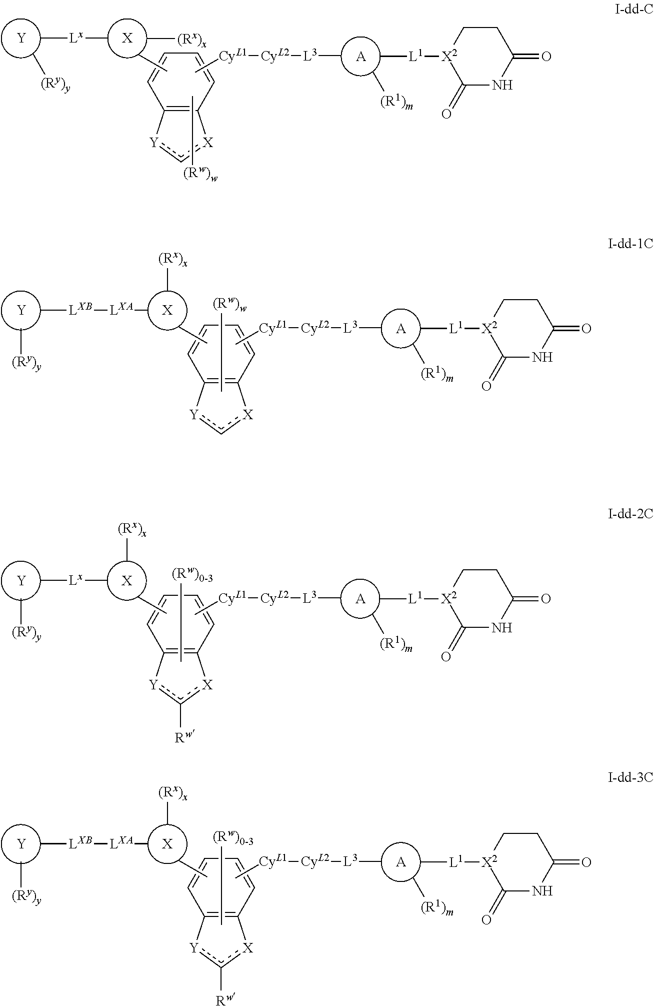

or a pharmaceutically acceptable salt thereof, wherein each variable is as defined above and described herein both individually and in combination.

In some embodiments, the present invention provides a compound of any one of the following formulae:

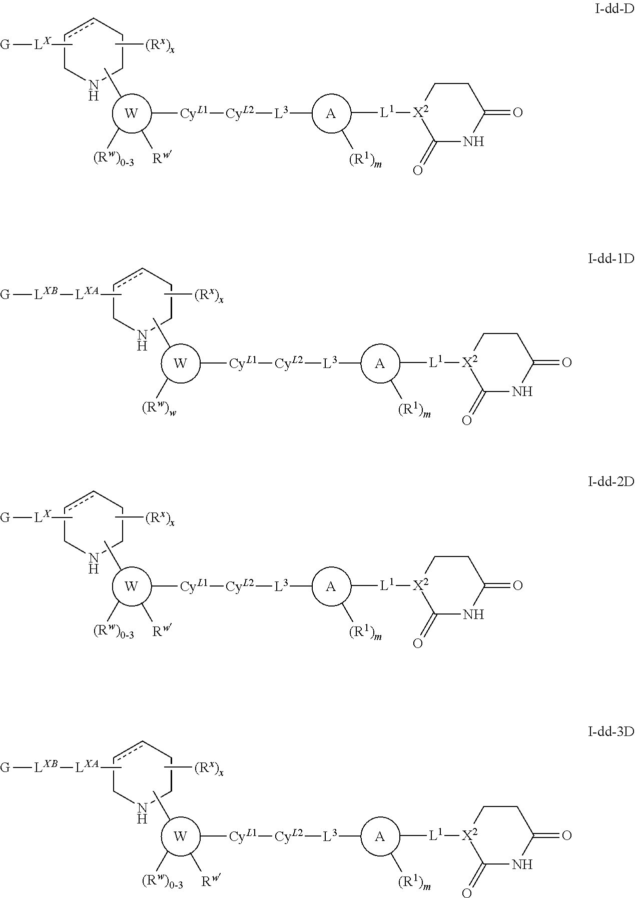

or a pharmaceutically acceptable salt thereof, wherein each variable is as defined above and described herein both individually and in combination.

In some embodiments, the present invention provides a compound of any one of the following formulae:

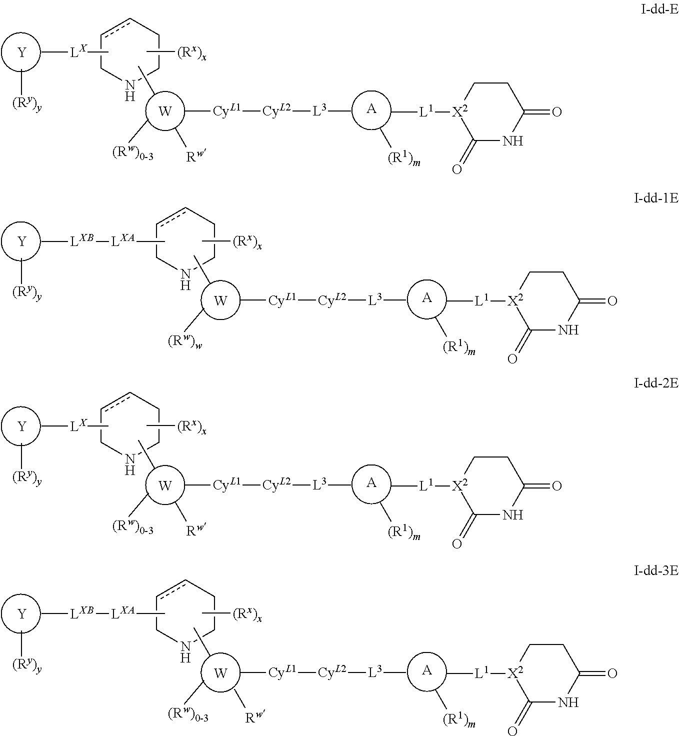

or a pharmaceutically acceptable salt thereof, wherein each variable is as defined above and described herein both individually and in combination.

In some embodiments, the present invention provides a compound of any one of the following formulae:

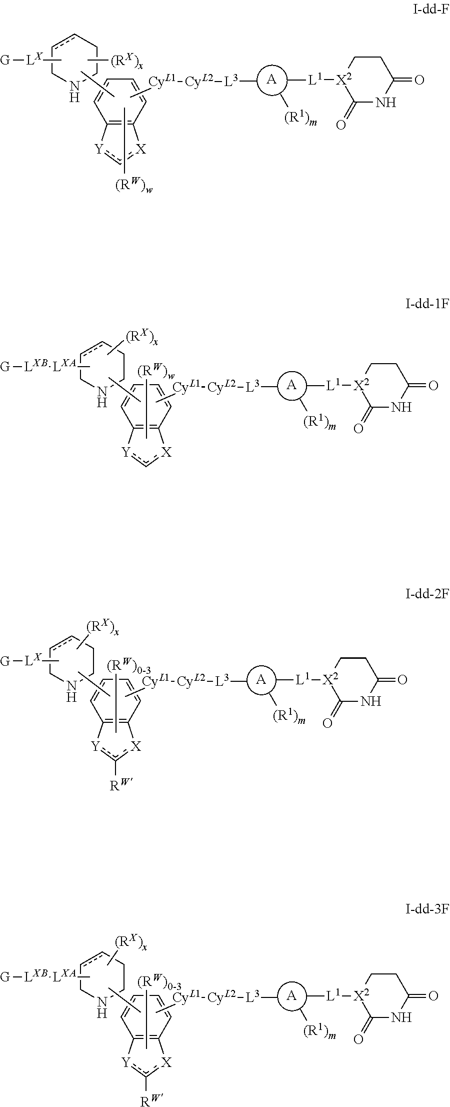

or a pharmaceutically acceptable salt thereof, wherein each variable is as defined above and described herein both individually and in combination.

In some embodiments, the present invention provides a compound of any one of the following formulae:

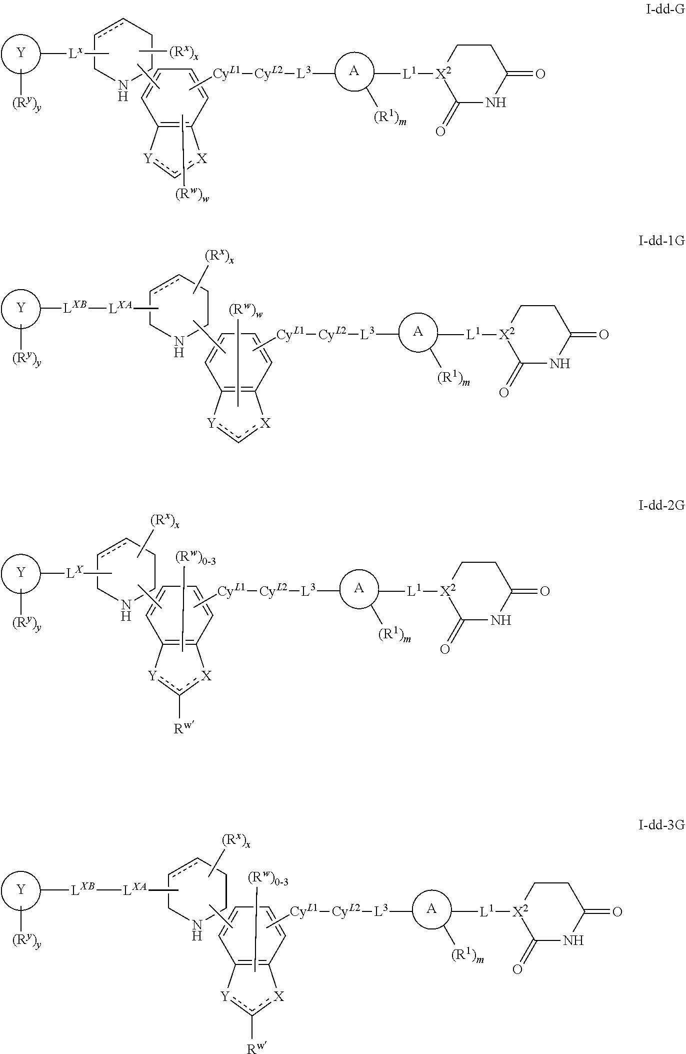

or a pharmaceutically acceptable salt thereof, wherein each variable is as defined above and described herein both individually and in combination.

In some embodiments, the present invention provides a compound of any one of the following formulae:

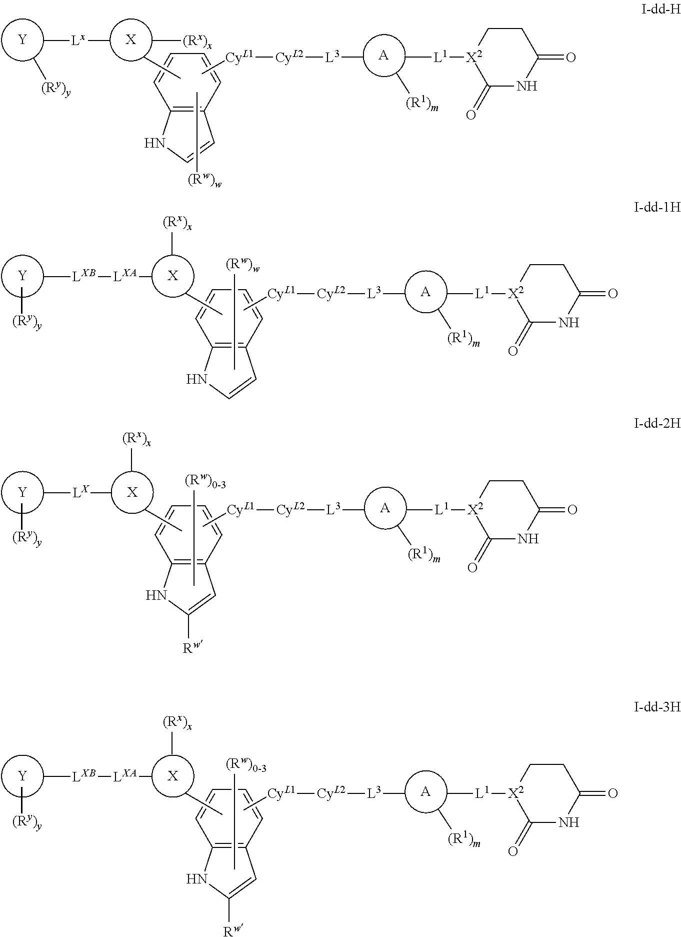

or a pharmaceutically acceptable salt thereof, wherein each variable is as defined above and described herein both individually and in combination.

In some embodiments, the present invention provides a compound of any one of the following formulae:

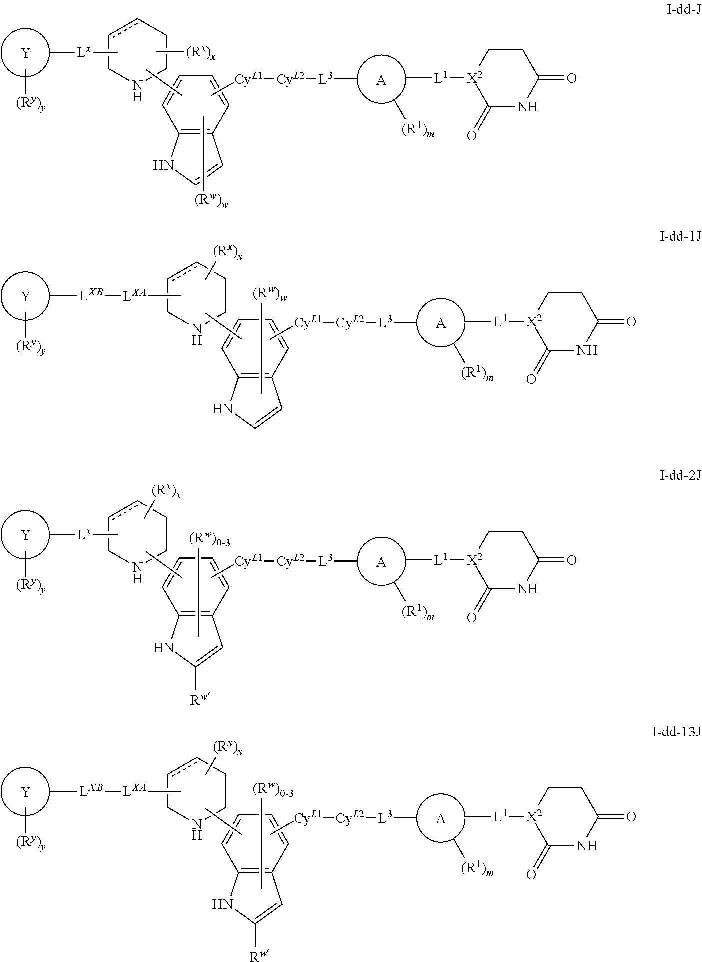

or a pharmaceutically acceptable salt thereof, wherein each variable is as defined above and described herein both individually and in combination.

In some embodiments of any one of formulae I-dd, I-dd-1, I-dd-2, I-dd-3, I-dd-A, I-dd-1A, I-dd-2A, I-dd-3A, I-dd-B, I-dd-1B, I-dd-2B, I-dd-3B. I-dd-C, I-dd-1C, I-dd-2C. I-dd-3C. I-dd-D, I-dd-1D, I-dd-2D, I-dd-3D, I-dd-E, I-dd-1E, I-dd-2E, I-dd-3E, I-dd-F, I-dd-1F, I-dd-2F, I-dd-3F. I-dd-G, I-dd-1G, I-dd-2G, I-dd-3G, I-dd-H, I-dd-1H, I-dd-2H, I-dd-3H, I-dd-J, I-dd-1J, I-dd-2J, or I-dd-3J, the structure

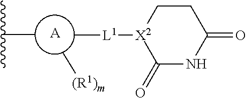

is of any one of formula I-aa-10′, I-aa-11′, I-aa-12′, I-aa-13′, or I-aa-14′:

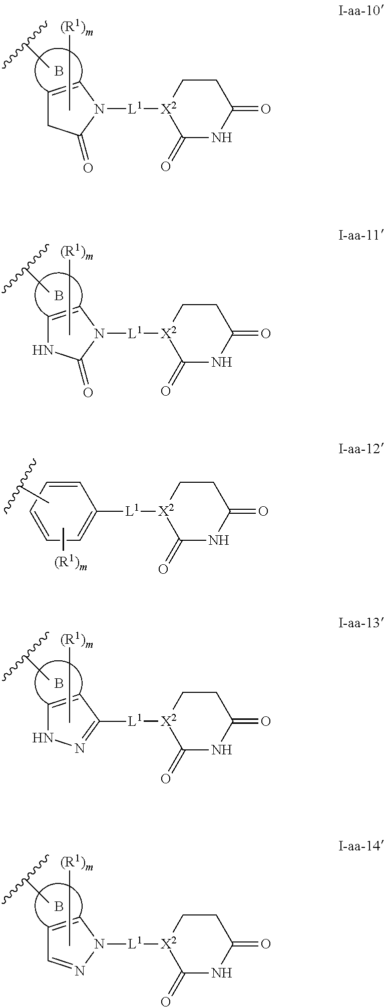

or a pharmaceutically acceptable salt thereof, wherein each variable is as defined above and described herein both individually and in combination.

In some embodiments of any one of formulae I-dd, I-dd-1, I-dd-2, I-dd-3, I-dd-A, I-dd-1A, I-dd-2A, I-dd-3A, I-dd-B, I-dd-1B, I-dd-2B, I-dd-3B, I-dd-C, I-dd-1C, I-dd-2C, I-dd-3C, I-dd-D, I-dd-1D, I-dd-2D, I-dd-3D, I-dd-E, I-dd-1E, I-dd-2E, I-dd-3E, I-dd-F, I-dd-1F, I-dd-2F, I-dd-3F, I-dd-G, I-dd-1G, I-dd-2G, I-dd-3G, I-dd-H, I-dd-1H, I-dd-2H, I-dd-3H, I-dd-J, I-dd-1J, I-dd-2J, or I-dd-3J, the structure

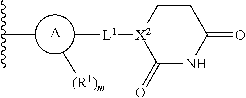

is of any one of formula I-aa-10a′, I-aa-11a′, I-aa-12a′, I-aa-12b′, I-aa-13a′, or I-aa-14a′:

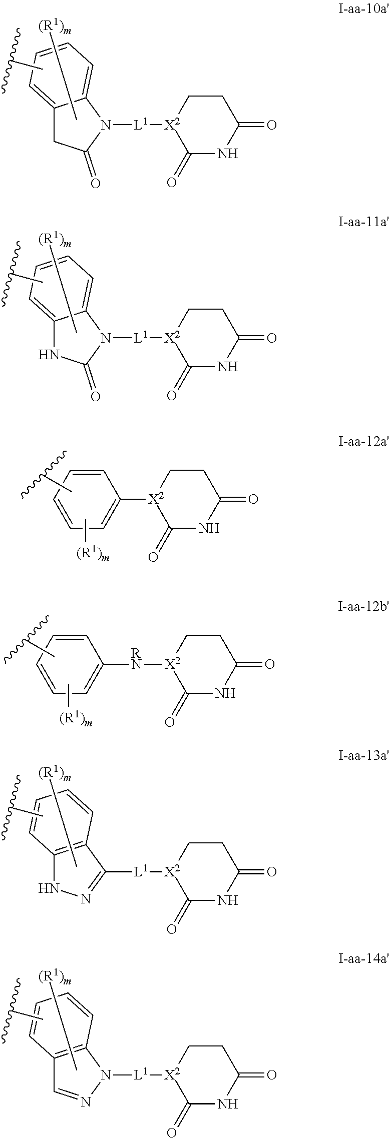

or a pharmaceutically acceptable salt thereof, wherein each variable is as defined above and described herein both individually and in combination.

Exemplary compounds of the invention are set forth in Table 1, below. It is understood that certain compounds herein may contain arbitrarily assigned stereochemistry or be derived from an intermediate with arbitrarily assigned stereochemistry. Unless otherwise stated, stereochemistry for compounds herein has been assigned arbitrarily, and it will be understood that any compound with arbitrarily assigned stereochemistry or produced from an intermediate with arbitrarily assigned stereochemistry may be depicted herein as a certain stereoisomer, but such compound may be the other stereoisomer (i.e., enantiomer or diastereomer). As shown in Table 1, column “EO”, for instances where a pair of stereoisomers were produced and the final compounds separated, the order of elution is provided: the first eluting isomer is indicated by “E1” and the second elution isomer is indicated by “E2”. As shown in Table 1, column “EO”, for instances where a pair of stereoisomers were produced, but a pair of intermediate stereoisomers were separated and further modified to arrive at the final compounds, the order of elution of the intermediate compound is provided: the first eluting isomer of the intermediate compound is indicated by “I1”, and the second eluting isomer of the intermediate compound is indicated by “I2.”

Lengthy table referenced here
US20260124309A1-20260507-T00001
Please refer to the end of the specification for access instructions.

In some embodiments, the present invention provides a compound set forth in Table 1, above, or a pharmaceutically acceptable salt thereof. In some embodiments, the present invention provides a compound set forth in Table 1, above.

In some embodiments, the present invention provides a pharmaceutical composition comprising a compound disclosed herein (described in embodiments herein, both singly and in combination), or a pharmaceutically acceptable salt thereof, together with a pharmaceutically acceptable carrier, adjuvant, or vehicle. For example, in some embodiments, the present invention provides a pharmaceutical composition comprising a compound of formula I as defined above, or a pharmaceutically acceptable salt thereof, together with a pharmaceutically acceptable carrier, adjuvant, or vehicle. In some embodiments, the present invention provides a pharmaceutical composition comprising a compound of formula I as defined above, together with a pharmaceutically acceptable carrier, adjuvant, or vehicle. In some embodiments, the present invention provides a pharmaceutical composition comprising a compound set forth in Table 1 above, or a pharmaceutically acceptable salt thereof, together with a pharmaceutically acceptable carrier, adjuvant, or vehicle. In some embodiments, the present invention provides a pharmaceutical composition comprising a compound set forth in Table 1 above, together with a pharmaceutically acceptable carrier, adjuvant, or vehicle.

In some embodiments, the present invention provides a compound described herein (such as a compound of formula I as defined above), or a pharmaceutically acceptable salt thereof, or a pharmaceutical composition comprising a compound described herein (such as a compound of formula I as defined above), or a pharmaceutically acceptable salt thereof, and a pharmaceutically acceptable carrier, adjuvant, or vehicle for use as a medicament.

In some embodiments, the invention also provides a compound described herein (such as a compound of formula I), or pharmaceutical compositions described herein, for use in a method for degrading STAT6 as described herein and/or in a method for treating a STAT6-mediated disorder as described herein. In some embodiments, the invention also provides a compound described herein (such as a compound of formula I), or pharmaceutical compositions described herein, for use in a method for degrading STAT6 as described herein. In some embodiments, the invention also provides a compound described herein (such as a compound of formula I), or pharmaceutical compositions described herein, for use in a method for treating a STAT6-mediated disorder as described herein.

In some embodiments, when a compound described herein (e.g., a compound of formula I) is administered to or contacted with a subject (e.g., a patient) or a cell (e.g., a peripheral blood mononuclear cell (PBMC)), the compound degrades STAT6, or a mutant thereof (e.g., STAT6 with gain of function (GOF) mutation(s)), in the subject or the cell. In some embodiments, the degradation of STAT6, or a mutant thereof, in the subject or the cell occurs through a novel ubiquitination pathway. For example, a subject or a cell treated or contacted with a compound described herein can produce a plurality of ubiquitinated STAT6, or mutants thereof, or fragments thereof, wherein the plurality of ubiquitinated STAT6, or mutants thereof, or fragments thereof has a ubiquitination profile that is distinct from a ubiquitination profile of a plurality of ubiquitinated STAT6, or mutants thereof, or fragments thereof from a subject or a cell that was not treated or contacted with the compound (e.g., the subject or the cell priorto administration of the compound). In some embodiments, a subject (e.g., patient) or a cell (e.g., a PBMC) treated or contacted with a compound described herein produces a change in the ubiquitin pathway of STAT6, or mutants thereof, in the subject or the cell relative to an untreated subject or an untreated cell. In some embodiments, when a biological sample from a subject (e.g., patient) or a cell (e.g., a PBMC) is treated or contacted with a compound described herein, the compound produces a novel composition of STAT6, or mutants thereof, or fragments thereof, wherein the novel composition of STAT6, or mutants thereof, or fragments thereof has a ubiquitination profile different (e.g., unique) relative to a ubiquitination profile of a composition of STAT6, or mutants thereof, or fragments thereof produced by an untreated subject or an untreated cell.

Accordingly, another aspect of the present disclosure relates to a polypeptide (e.g., STAT6) that is ubiquitinated. In some embodiments, a polypeptide is ubiquitinated at one or more lysine residues. In some embodiments, a polypeptide is polyubiquitinated at one or more lysine residues. Ubiquitination of a polypeptide involves covalent attachment of ubiquitin to the polypeptide via an isopeptide bond. In some embodiments, ubiquitination of a polypeptide comprises covalent attachment of ubiquitin to an epsilon amino group of a lysine residue. In some embodiments, polyubiquitination of a polypeptide comprises covalent attachment of more than one ubiquitin to an epsilon amino group of a lysine residue of the polypeptide. In some embodiments, polyubiquitination of a polypeptide comprises covalent attachment of more than one ubiquitin to epsilon amino groups of one or more lysine residues of the polypeptide.

Another aspect of the present disclosure relates to a polypeptide comprising SEQ ID NO: 1, wherein the polypeptide is ubiquitinated at one or more of the following lysine residues: lysine 647 (K⁶⁴⁷), lysine 595 (K⁵⁹⁵), lysine 504 (K⁵⁰⁴), lysine 319 (K³¹⁹), lysine 307 (K³⁰⁷), and lysine 288 (K²⁸⁸). In some embodiments, the polypeptide is polyubiquitinated at one or more of the following lysine residues: lysine 647 (K⁶⁴⁷), lysine 595 (K⁵⁹⁵), lysine 504 (K⁵⁰⁴), lysine 319 (K³¹⁹), lysine 307 (K³⁰⁷), and lysine 288 (K²⁸⁸).

In some embodiments, the polypeptide is ubiquitinated at lysine 647 (K⁶⁴⁷). In some embodiments, the polypeptide is ubiquitinated at lysine 595 (K⁵⁹⁵). In some embodiments, the polypeptide is ubiquitinated at lysine 504 (K⁵⁰⁴). In some embodiments, the polypeptide is ubiquitinated at lysine 319 (K³¹⁹). In some embodiments, the polypeptide is ubiquitinated at lysine 307 (K³⁰⁷). In some embodiments, the polypeptide is ubiquitinated at lysine 288 (K²⁸⁸).

In some embodiments, the polypeptide is ubiquitinated at two or more of the following lysine residues: lysine 647 (K⁶⁴⁷), lysine 595 (K⁵⁹⁵), lysine 504 (K⁵⁰⁴), lysine 319 (K³¹⁹), lysine 307 (K³⁹⁷), and lysine 288 (K²⁸⁸).

In some embodiments, the polypeptide is polyubiquitinated at two or more of the following lysine residues: lysine 647 (K⁶⁴⁷), lysine 595 (K⁵⁹⁵), lysine 504 (K⁵⁰⁴), lysine 319 (K³¹⁹), lysine 307 (K³⁰⁷) and lysine 288 (K²⁸⁸).

In some embodiments, the polypeptide is ubiquitinated at three or more of the following lysine residues: lysine 647 (K⁶⁴⁷), lysine 595 (K⁵⁹⁵), lysine 504 (K⁵⁰⁴), lysine 319 (K³¹⁹), lysine 307 (K³⁹⁷), and lysine 288 (K²⁸⁸). In some embodiments, the polypeptide is polyubiquitinated at three or more of the following lysine residues: lysine 647 (K⁶⁴⁷), lysine 595 (K⁵⁹⁵), lysine 504 (K⁵⁰⁴), lysine 319 (K³¹⁹), lysine 307 (K³⁰⁷), and lysine 288 (K²⁸⁸).

In some embodiments, the polypeptide is ubiquitinated at four or more of the following lysine residues: lysine 647 (K⁶⁴⁷), lysine 595 (K⁵⁹⁵), lysine 504 (K⁵⁰⁴), lysine 319 (K³¹⁹), lysine 307 (K³⁰⁷), and lysine 288 (K²⁸⁸). In some embodiments, the polypeptide is polyubiquitinated at four or more of the following lysine residues: lysine 647 (K⁶⁴⁷), lysine 595 (K⁵⁹⁵), lysine 504 (K⁵⁰⁴), lysine 319 (K³¹⁹), lysine 307 (K³⁰⁷), and lysine 288 (K²⁸⁸).

In some embodiments, the polypeptide is ubiquitinated at five or more of the following lysine residues: lysine 647 (K⁶⁴⁷), lysine 595 (K⁵⁹⁵), lysine 504 (K⁵⁰⁴), lysine 319 (K³¹⁹), lysine 307 (K³⁰⁷), and lysine 288 (K²⁸⁸). In some embodiments, the polypeptide is polyubiquitinated at five or more of the following lysine residues: lysine 647 (K⁶⁴⁷), lysine 595 (K⁵⁹⁵), lysine 504 (K⁵⁰⁴), lysine 319 (K³¹⁹), lysine 307 (K³⁰⁷), and lysine 288 (K²⁸⁸).

In some embodiments, the polypeptide is ubiquitinated at the following lysine residues: lysine 647 (K⁶⁴⁷), lysine 595 (K¹⁹⁵), lysine 504 (K⁵⁰⁴), lysine 319 (K³¹⁹), lysine 307 (K³⁰⁷), and lysine 288 (K²⁸⁸). In some embodiments, the polypeptide is polyubiquitinated at each of the following lysine residues: lysine 647 (K⁶⁴⁷), lysine 595 (K⁵⁹⁵), lysine 504 (K⁵⁰⁴), lysine 319 (K³¹⁹), lysine 307 (K³07), and lysine 288 (K²⁸⁸).

In some embodiments, the polypeptide is further ubiquitinated at one or more of the following lysine residues: lysine 618 (K⁶¹⁸), lysine 614 (K⁶¹⁴), lysine 374 (K³⁷⁴), and lysine 277 (K²⁷⁷). In some embodiments, the polypeptide is further polyubiquitinated at one or more of the following lysine residues: lysine 618 (K⁶¹⁸), lysine 614 (K⁶¹⁴), lysine 374 (K³⁷⁴), and lysine 277 (K²⁷⁷). In some embodiments, the polypeptide is further ubiquitinated at lysine 618 (K⁶¹⁸). In some embodiments, the polypeptide is further ubiquitinated at lysine 614 (K⁶¹⁴). In some embodiments, the polypeptide is further ubiquitinated at lysine 374 (K³⁷⁴). In some embodiments, the polypeptide is further ubiquitinated at lysine 277 (K²⁷⁷).

In some embodiments, the polypeptide is further ubiquitinated at two or more of one or more of the following lysine residues: lysine 618 (K⁶¹⁸), lysine 614 (K⁶¹⁴), lysine 374 (K³⁷⁴), and lysine 277 (K²⁷⁷). In some embodiments, the polypeptide is further polyubiquitinated at two or more of the following lysine residues: lysine 618 (K⁶¹⁸), lysine 614 (K⁶¹⁴), lysine 374 (K³⁷⁴), and lysine 277 (K²⁷⁷).

In some embodiments, the polypeptide is further ubiquitinated at three or more of one or more of the following lysine residues: lysine 618 (K⁶¹⁸), lysine 614 (K⁶¹⁴), lysine 374 (K³⁷⁴), and lysine 277 (K²⁷⁷). In some embodiments, the polypeptide is further polyubiquitinated at three or more of the following lysine residues: lysine 618 (K⁶¹⁸), lysine 614 (K⁶¹⁴), lysine 374 (K³⁷⁴), and lysine 277 (K²⁷⁷).

In some embodiments, the polypeptide is further ubiquitinated at lysine 618 (K⁶¹⁸) lysine 614 (K⁶¹⁴), lysine 374 (K³⁷⁴), and lysine 277 (K²⁷⁷). In some embodiments, the polypeptide is further polyubiquitinated at lysine 618 (K^{61′}), lysine 614 (K⁶¹⁴), lysine 374 (K³⁷⁴), and lysine 277 (K²⁷⁷).

In some embodiments, the polypeptide is further ubiquitinated at one or more of the following lysine residues: lysine 636 (K⁶³), lysine 630 (K⁶³⁰), lysine 284 (K²⁸⁴), lysine 124 (K¹²⁴), lysine 108 (K¹⁰⁸), lysine 107 (K¹⁰⁷), and lysine 94 (K⁹⁴). In some embodiments, the polypeptide is further polyubiquitinated atone or more of the following lysine residues: lysine 636 (K⁶³⁶), lysine 630 (K⁶³⁰), lysine 284 (K²⁸⁴), lysine 124 (K¹²⁴), lysine 108 (K¹⁰⁸), lysine 107 (K¹⁰⁷), and lysine 94 (K⁹⁴). In some embodiments, the polypeptide is further ubiquitinated at lysine 636 (K⁶³⁶). In some embodiments, the polypeptide is further ubiquitinated at lysine 630 (K⁶³⁰). In some embodiments, the polypeptide is further ubiquitinated at lysine 284 (K²⁸⁴). In some embodiments, the polypeptide is further ubiquitinated at lysine 124 (K¹²⁴). In some embodiments, the polypeptide is further ubiquitinated at lysine 108 (K¹⁰⁸). In some embodiments, the polypeptide is further ubiquitinated at lysine 107 (K¹⁰⁷). In some embodiments, the polypeptide is further ubiquitinated at lysine 94 (K¹⁴).

In some embodiments, the polypeptide is further ubiquitinated at two or more of the following lysine residues: lysine 636 (K⁶³⁶), lysine 630 (K⁶³⁰), lysine 284 (K²⁸⁴), lysine 124 (K¹²⁴), lysine 108 (K¹⁰⁸), lysine 107 (K¹⁰⁷), and lysine 94 (K⁹⁴). In some embodiments, the polypeptide is further polyubiquitinated at two or more of the following lysine residues: lysine 636 (K⁶³⁶), lysine 630 (K⁶³⁰), lysine 284 (K²⁸⁴), lysine 124 (K¹²⁴), lysine 108 (K¹⁰⁸), lysine 107 (K¹⁰⁷), and lysine 94 (K¹⁴).

In some embodiments, the polypeptide is further ubiquitinated at three or more of the following lysine residues: lysine 636 (K⁶³⁶), lysine 630 (K⁶³⁰), lysine 284 (K²⁸⁴), lysine 124 (K¹²⁴), lysine 108 (K¹⁰⁸), lysine 107 (K¹⁰⁷), and lysine 94 (K⁹⁴). In some embodiments, the polypeptide is further polyubiquitinated at three or more of the following lysine residues: lysine 636 (K⁶³⁶), lysine 630 (K⁶³⁰) lysine 284 (K²⁸⁴), lysine 124 (K¹²⁴), lysine 108 (K¹⁰⁸), lysine 107 (K¹⁰⁷), and lysine 94 (K⁹⁴).

In some embodiments, the polypeptide is further ubiquitinated at four or more of the following lysine residues: lysine 636 (K⁶³⁶), lysine 630 (K⁶³⁰), lysine 284 (K²84), lysine 124 (K¹²⁴), lysine 108 (K¹⁰⁸), lysine 107 (K¹⁰⁷), and lysine 94 (K⁹⁴). In some embodiments, the polypeptide is further polyubiquitinated at four or more of the following lysine residues: lysine 636 (K⁶³⁶), lysine 630 (K⁶³⁰), lysine 284 (K²⁸⁴), lysine 124 (K¹²⁴), lysine 108 (K¹⁰⁸), lysine 107 (K¹⁰⁷), and lysine 94 (K⁹⁴).

In some embodiments, the polypeptide is further ubiquitinated at five or more of the following lysine residues: lysine 636 (K⁶³⁶), lysine 630 (K⁶³⁰), lysine 284 (K²⁸⁴), lysine 124 (K¹²⁴), lysine 108 (K¹⁰⁸), lysine 107 (K¹⁰⁷), and lysine 94 (K⁹⁴). In some embodiments, the polypeptide is further polyubiquitinated at five or more of the following lysine residues: lysine 636 (K⁶³⁶), lysine 630 (K⁶³⁰), lysine 284 (K²⁸⁴), lysine 124 (K¹²⁴), lysine 108 (K¹⁰⁸), lysine 107 (K¹⁰⁷), and lysine 94 (K⁹⁴).

In some embodiments, the polypeptide is further ubiquitinated at six or more of the following lysine residues: lysine 636 (K⁶³⁶), lysine 630 (K⁶³⁰), lysine 284 (K²⁸⁴), lysine 124 (K¹²⁴), lysine 108 (K¹⁰⁸), lysine 107 (K¹⁰⁷), and lysine 94 (K⁹⁴). In some embodiments, the polypeptide is further polyubiquitinated at six or more of the following lysine residues: lysine 636 (K⁶³⁶), lysine 630 (K⁶³⁰), lysine 284 (K²⁸⁴), lysine 124 (K¹²⁴), lysine 108 (K¹⁰⁸), lysine 107 (K¹⁰⁷), and lysine 94 (K⁹⁴).

In some embodiments, the polypeptide is further ubiquitinated at lysine 636 (K⁶³6), lysine 630 (K⁶³⁰), lysine 284 (K²⁸⁴), lysine 124 (K²⁴), lysine 108 (K¹⁰⁸), lysine 107 (K¹⁰⁷), and lysine 94 (K⁹⁴). In some embodiments, the polypeptide is further polyubiquitinated at each of lysine 636 (K⁶³⁶), lysine 630 (K⁶³⁰), lysine 284 (K²⁸⁴), lysine 124 (K¹²⁴), lysine 108 (K¹⁰⁸), lysine 107 (K¹⁰⁷), and lysine 94 (K⁹⁴).

In some embodiments, the polypeptide is from a sample. In some embodiments, the sample comprises a biological sample. In some embodiments, the comprises a lysate. In some embodiments, the sample comprises a peripheral blood mononuclear cell (PBMC).

Another aspect of the present disclosure relates to a plurality of polypeptides comprising at least one of any of the foregoing ubiquitinated polypeptides.

Another aspect of the present disclosure relates to a plurality of polypeptides comprising one or more of any of the foregoing ubiquitinated polypeptides.

In some embodiments, the plurality of polypeptides comprises at least one polypeptide ubiquitinated at a first lysine residue, at least one polypeptide ubiquitinated at a second lysine residue, or at least one polypeptide ubiquitinated at a first lysine residue and at a second lysine residue.

In some embodiments, the first lysine residue is lysine 647 (K⁶⁴⁷). In some embodiments, the first lysine residue is lysine 595 (K⁵⁹⁵). In some embodiments, the first lysine residue is lysine 504 (K⁵⁰⁴). In some embodiments, the first lysine residue is lysine 319 (K³¹⁹). In some embodiments, the first lysine residue is lysine 307 (K³⁰⁷). In some embodiments, the first lysine residue is lysine 288 (K²⁸⁸).

In some embodiments, the second lysine residue is lysine 636 (K⁶³⁶). In some embodiments, the second lysine residue is lysine 630 (K⁶³⁰). In some embodiments, the second lysine residue is lysine 284 (K²⁸⁴). In some embodiments, the second lysine residue is lysine 124 (K¹²⁴). In some embodiments, the second lysine residue is lysine 108 (K¹⁰⁸). In some embodiments, the second lysine residue is lysine 107 (K¹⁰⁷). In some embodiments, the second lysine residue is lysine 94 (K⁹⁴).

In some embodiments, the plurality of polypeptides is measured using electrospray ionization mass spectrometry.

In some embodiments, the plurality of polypeptides has a mass spectrum that indicates a signal intensity ratio of about 2:1, about 2.1:1, about 2.2:1, about 2.3:1, about 2.4:1, about 2.5:1, about 2.6:1, about 2.7:1, about 2.8:1, about 2.9:1, about 3:1, about 3.1:1, about 3.2:1, about 3.3:1, about 3.4:1, about 3.5:1, about 3.6:1, about 3.7:1, about 3.8:1, about 3.9:1, about 4:1, about 4.1:1, about 4.2:1, about 4.3:1, about 4.4:1, about 4.5:1, about 4.6:1, about 4.7:1, about 4.8:1, about 4.9:1, about 5:1, about 5.1:1, about 5.2:1, about 5.3:1, about 5.4:1, about 5.5:1, about 5,6:1, about 5.7:1, about 5.8:1, about 5.9:1, about 6:1, about 6.1:1, about 6.2:1, about 6.3:1, about 6.4:1, about 6.5:1, about 6.6:1, about 6.7:1, about 6.8:1, about 6.9:1, about 7:1, about 7.1:1, about 7.2:1, about 7.3:1, about 7.4:1, about 7.5:1, about 7.6:1, about 7.7:1, about 7.8:1, about 7.9:1, about 8:1, about 8.1:1, about 8.2:1, about 8.3:1, about 8.4:1, about 8.5:1, about 8.6:1, about 8.7:1, about 8.8:1, about 8.9:1, about 9:1, about 9.1:1, about 9.2:1, about 9.3:1, about 9.4:1, about 9.5:1, about 9.6:1, about 9.7:1, about 9.8:1, about 9.9:1, or about 10:1 for the plurality of polypeptides ubiquitinated at the first lysine residue to the plurality of polypeptides ubiquitinated at the second lysine residue. In some embodiments, the plurality of polypeptides has a mass spectrum that indicates a signal intensity ratio of about 10:1, about 10.5:1, about 11:1, about 11.5:1, about 12:1, about 12.5:1, about 13:1, about 13.5:1, about 14:1, about 14.5:1, about 15:1, about 15.5:1, about 16:1, about 16.5:1, about 17:1, about 17.5:1, about 18:1, about 18.5:1, about 19:1, about 19.5:1, or about 20:1 for the plurality of polypeptides ubiquitinated at the first lysine residue to the plurality of polypeptides ubiquitinated at the second lysine residue.

In some embodiments, the plurality of polypeptides has a mass spectrum that indicates a signal intensity ratio of about 20:1, about 25:1, about 30:1, about 35:1, about 40:1, about 45:1, about 50:1, about 55:1, about 60:1, about 65:1, about 70:1, about 75:1, about 80:1, about 85:1, about 90:1, about 95:1, about 100:1, or about 105:1 for the plurality of polypeptides ubiquitinated at the first lysine residue to the plurality of polypeptides ubiquitinated at the second lysine residue.

In some embodiments, the plurality of polypeptides has a mass spectrum that indicates a signal intensity ratio of about 125:1, 130:1, about 135:1, about 140:1, about 145:1, about 150:1, or about 155:1 for the plurality of polypeptides ubiquitinated at the first lysine residue to the plurality of polypeptides ubiquitinated at the second lysine residue.

In some embodiments, the plurality of polypeptides has a mass spectrum that indicates a signal intensity ratio of about 190:1, about 192:1, about 194:1, about 196:1, about 198:1, about 200:1, about 202:1, about 204:1, about 206:1, about 208:1, about 210:1, about 212:1, about 214:1, about 216:1, about 218:1, about 220:1, about 222:1, about 224:1, about 226:1, about 228:1, about 230:1, about 232:1, about 234:1, about 236:1, about 238:1, about 240:1, about 242:1, about 244:1, about 246:1, about 248:1, or about 250:1 for the plurality of polypeptides ubiquitinated at the first lysine residue to the plurality of polypeptides ubiquitinated at the second lysine residue.

In some embodiments, the plurality of polypeptides has a mass spectrum that indicates a signal intensity ratio of about 300:1, about 305:1, about 310:1, about 315:1, or about 320:1 for the plurality of polypeptides ubiquitinated at the first lysine residue to the plurality of polypeptides ubiquitinated at the second lysine residue.

In some embodiments, the plurality of polypeptides has a mass spectrum that indicates a signal intensity ratio of about 365:1, about 370:1, about 375:1, about 380:1, or about 385:1 for the plurality of polypeptides ubiquitinated at the first lysine residue to the plurality of polypeptides ubiquitinated at the second lysine residue.

In some embodiments, the plurality of polypeptides has a mass spectrum that indicates a signal intensity ratio of about 1:1, about 1:1.1, about 1:1.2, about 1:1.3, about 1:1.4, about 1:1.5, about 1:1.6, about 1:1.7, about 1:1.8, about 1:1.9, about 1:2, about 1:2.1, about 1:2.2, about 1:2.3, about 1:2.4, about 1:2.5, about 1:2.6, about 1:2.7, about 1:2.8, about 1:2.9, about 1:3, about 1:3.1, about 1:3.2, about 1:3.3, about 1:3.4, about 1:3.5, about 1:3.6, about 1:3.7, about 1:3.8, about 1:3.9, about 1:4, about 1:4.1, about 1:4.2, about 1:4.3, about 1:4.4, about 1:4.5, about 1:4.6, about 1:4.7, about 1:4.8, about 1:4.9, about 1:5, about 1:5.1, about 1:5.2, about 1:5.3, about 1:5.4, about 1:5.5, about 1:5,6, about 1:5.7, about 1:5.8, about 1:5.9, about 1:6, about 1:6.1, about 1:6.2, about 1:6.3, about 1:6.4, about 1:6.5, about 1:6.6, about 1:6.7, about 1:6.8, about 1:6.9, about 1:7, about 1:7.1, about 1:7.2, about 1:7.3, about 1:7.4, about 1:7.5, about 1:7.6, about 1:7.7, about 1:7.8, about 1:7.9, about 1:8, about 1:8.1, about 1:8.2, about 1:8.3, about 1:8.4, about 1:8.5, about 1:8.6, about 1:8.7, about 1:8.8, about 1:8.9, about 1:9, about 1:9.1, about 1:9.2, about 1:9.3, about 1:9.4, about 1:9.5, about 1:9.6, about 1:9.7, about 1:9.8, about 1:9.9, about 1:10, about 1:10.1, about 1:10.2, about 1:10.3, about 1:10.4, about 1:10.5, about 1:10.6, about 1:10.7, about 1:10.8, about 1:10.9, about 1:11, about 1:11.1, about 1:11.2, about 1:11.3, about 1:11.4, about 1:11.5, about 1:11.6, about 1:11.7, about 1:11.8, about 1:11.9, about 1:12, about 1:12.1, about 1:12.2, about 1:12.3, about 1:12.4, about 1:12.5, about 1:12.6, about 1:12.7, about 1:12.8, about 1:12.9, about 1:13, about 1:13.1, about 1:13.2, about 1:13.3, about 1:13.4, about 1:13.5, about 1:13.6, about 1:13.7, about 1:13.8, about 1:13.9, about 1:14, about 1:14.1, about 1:14.2, about 1:14.3, about 1:14.4, about 1:14.5, about 1:14.6, about 1:14.7, about 1:14.8, about 1:14.9, about 1:15, about 1:15.1, about 1:15.2, about 1:15.3, about 1:15.4, about 1:15.5, about 1:15.6, about 1:15.7, about 1:15.8, about 1:15.9, about 1:16, about 1:16.1, about 1:16.2, about 1:1165.3, about 1:16.4, about 1:16.5, about 1:16.6, about 1:16.7, about 1:16.8, about 1:16.9, about 1:17, about 1:17.1, about 1:17.2, about 1:17.3, about 1:17.4, about 1:17.5, about 1:17.6, about 1:17.7, about 1:17.8, about 1:17.9, about 1:18, about 1:18.1, about 1:18.2, about 1:18.3, about 1:18.4, about 1:18.5, about 1:18.6, about 1:18.7, about 1:18.8, about 1:18.9, about 1:19, about 1:19.1, about 1:19.2, about 1:19.3, about 1:19.4, or about 1:19.5 for the plurality of polypeptides ubiquitinated at the first lysine residue to the plurality of polypeptides ubiquitinated at the second lysine residue.

In some embodiments, the plurality of polypeptides has a mass spectrum that indicates that about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 22%, about 24%, about 26%, about 28%, about 30%, about 32%, about 34%, about 36%, about 38%, about 40%, about 42%, about 44%, about 46%, about 48%, or about 50% of ubiquitination is at the first lysine residue.

Accordingly, another aspect of the present disclosure relates to a plurality of polypeptides or fragments thereof (e.g., a plurality of STAT6, or mutants thereof, or fragments thereof, or a plurality of polypeptides or fragments thereof, wherein each of the polypeptides or fragments thereof comprises a sequence that shares about 85% identity to about 100% identity (e.g., about 85% identity, about 86% identity, about 87% identity, about 88% identity, about 89% identity, about 90% identity, about 91% identity, about 92% identity, about 93% identity, about 94% identity, about 95% identity, about 96% identity, about 97% identity, about 98% identity, about 99% identity, or about 100% identity) with an equal length portion of SEQ ID NO: 1), wherein the plurality of polypeptides or fragments thereof has a novel ubiquitination profile (e.g., a ubiquitination profile described herein, or any combination thereof).

Another aspect of the present disclosure relates to a compound (e.g., a degrader and/or an inhibitor of STAT6, or a mutant thereof (e.g., a compound described herein, such as a compound of formula I)), wherein when the compound is administered to or contacted with a subject (e.g., a patient) or a cell (e.g., a PBMC), the compound produces a plurality of polypeptides or fragments thereof (e.g., a plurality of STAT6, or mutants thereof, or fragments thereof; or a plurality of polypeptides or fragments thereof, wherein each of the polypeptides or fragments thereof comprises a sequence that shares about 85% identity to about 100% identity (e.g., about 85% identity, about 86% identity, about 87% identity, about 88% identity, about 89% identity, about 90% identity, about 91% identity, about 92% identity, about 93% identity, about 94% identity, about 95% identity, about 96% identity, about 97% identity, about 98% identity, about 99% identity, or about 100% identity) having a novel ubiquitination profile (e.g., a ubiquitination profile described herein, or any combination thereof).

Another aspect of the present disclosure relates to a method of producing a plurality of polypeptides or fragments thereof (e.g., a plurality of STAT6, or mutants thereof, or fragments thereof; or a plurality of polypeptides or fragments thereof, wherein each of the polypeptides or fragments thereof comprises a sequence that shares about 85% identity to about 100% identity (e.g., about 85% identity, about 86% identity, about 87% identity, about 88% identity, about 89% identity, about 90% identity, about 91% identity, about 92% identity, about 93% identity, about 94% identity, about 95% identity, about 96% identity, about 97% identity, about 98% identity, about 99% identity, or about 100% identity) having a novel ubiquitination profile (e.g., a ubiquitination profile described herein, or any combination thereof), wherein the method comprises:
(a) administering a compound (e.g., a degrader and/or an inhibitor of STAT6 protein, or a mutant thereof (e.g., a compound described herein, such as a compound of formula I)), to a subject (e.g., a patient) or a cell (e.g., a PBMC):
(b) lysing (e.g., digesting) a sample (e.g., biological sample) from the subject or lysing (e.g., digesting) the cell; and
(c) determining the ubiquitination profile of the sample by spectroscopy (e.g., mass spectroscopy).

In some embodiments, the lysing of the sample or the cell comprises digesting the sample or the cell with an enzyme. In some embodiments, the enzyme is a peptide digestion enzyme. In some embodiments, the enzyme is trypsin.

In some embodiments, the lysing of the sample or the cell occurs at half the time needed to reduce STAT6 levels by half as compared to a steady state level in a subject or a cell without treatment with the compound. In some embodiments, the lysing of the sample or the cell occurs at a timepoint of about 10 minutes, about 11 minutes, about 12 minutes, about 13 minutes, about 14 minutes, about 15 minutes, about 16 minutes, about 17 minutes, about 18 minutes, about 19 minutes, about 20 minutes, about 21 minutes, about 22 minutes, about 23 minutes, about 24 minutes, about 25 minutes, about 26 minutes, about 27 minutes, about 28 minutes, about 29 minutes, about 30 minutes, about 35 minutes, about 40 minutes, about 45 minutes, about 50 minutes, about 55 minutes, about 1 hour, about 1.5 hours, about 2 hours, about 2.5 hour, about 5 hours, about 10 hours, about 12 hours, or about 24 hours, after administering the compound to the subject or the cell.

In some embodiments, the ubiquitination profile of the plurality of polypeptides or fragments thereof comprises:
(i) ubiquitination at a first lysine residue, a second lysine residue, a third lysine residue, or a fourth lysine residue, or any combinations thereof; and
(ii) ubiquitination at a fifth lysine residue, a sixth lysine residue, a seventh lysine residue, or an eighth lysine residue, or any combinations thereof.

In some embodiments, the ubiquitination profile of the plurality of polypeptides or fragments thereof comprises:
(i) ubiquitination at a first lysine residue; and
(ii) ubiquitination at a fifth lysine residue.

In some embodiments, the ubiquitination profile of the plurality of polypeptides or fragments thereof comprises:
(i) ubiquitination at a first lysine residue; and
(ii) ubiquitination at a sixth lysine residue.

In some embodiments, the ubiquitination profile of the plurality of polypeptides or fragments thereof comprises:
(i) ubiquitination at a first lysine residue; and
(ii) ubiquitination at a seventh lysine residue.

In some embodiments, the ubiquitination profile of the plurality of polypeptides or fragments thereof comprises:
(i) ubiquitination at a first lysine residue; and
(ii) ubiquitination at an eighth lysine residue.

In some embodiments, the ubiquitination profile of the plurality of polypeptides or fragments thereof comprises:
(i) ubiquitination at a second lysine residue; and
(ii) ubiquitination at a fifth lysine residue.

In some embodiments, the ubiquitination profile of the plurality of polypeptides or fragments thereof comprises:
(i) ubiquitination at a second lysine residue; and
(ii) ubiquitination at a sixth lysine residue.

In some embodiments, the ubiquitination profile of the plurality of polypeptides or fragments thereof comprises:
(i) ubiquitination at a second lysine residue; and
(ii) ubiquitination at a seventh lysine residue.

In some embodiments, the ubiquitination profile of the plurality of polypeptides or fragments thereof comprises:
(i) ubiquitination at a second lysine residue; and
(ii) ubiquitination at an eighth lysine residue.

In some embodiments, the ubiquitination profile of the plurality of polypeptides or fragments thereof comprises:
(i) ubiquitination at a third lysine residue; and
(ii) ubiquitination at a fifth lysine residue.

In some embodiments, the ubiquitination profile of the plurality of polypeptides or fragments thereof comprises:
(i) ubiquitination at a third lysine residue; and
(ii) ubiquitination at a sixth lysine residue.

In some embodiments, the ubiquitination profile of the plurality of polypeptides or fragments thereof comprises:
(i) ubiquitination at a third lysine residue; and
(ii) ubiquitination at a seventh lysine residue.

In some embodiments, the ubiquitination profile of the plurality of polypeptides or fragments thereof comprises:
(i) ubiquitination at a third lysine residue; and
(ii) ubiquitination at an eighth lysine residue.

In some embodiments, the ubiquitination profile of the plurality of polypeptides or fragments thereof comprises:
(i) ubiquitination at a fourth lysine residue; and
(ii) ubiquitination at a fifth lysine residue.

In some embodiments, the ubiquitination profile of the plurality of polypeptides or fragments thereof comprises:
(i) ubiquitination at a fourth lysine residue; and
(ii) ubiquitination at a sixth lysine residue.

In some embodiments, the ubiquitination profile of the plurality of polypeptides or fragments thereof comprises:
(i) ubiquitination at a fourth lysine residue; and
(ii) ubiquitination at a seventh lysine residue.

In some embodiments, the ubiquitination profile of the plurality of polypeptides or fragments thereof comprises:
(i) ubiquitination at a fourth lysine residue; and
(ii) ubiquitination at an eighth lysine residue.

In some embodiments, the ubiquitination at a first lysine residue, a second lysine residue, a third lysine residue, or a fourth lysine residue, or any combinations thereof comprises ubiquitination at the first lysine residue and the second lysine residue. In some embodiments, the ubiquitination at a first lysine residue, a second lysine residue, a third lysine residue, or a fourth lysine residue, or any combinations thereof comprises ubiquitination at the first lysine residue and the third lysine residue. In some embodiments, the ubiquitination at a first lysine residue, a second lysine residue, a third lysine residue, or a fourth lysine residue, or any combinations thereof comprises ubiquitination at the first lysine residue and the fourth lysine residue. In some embodiments, the ubiquitination at a first lysine residue, a second lysine residue, a third lysine residue, or a fourth lysine residue, or any combinations thereof comprises ubiquitination at the second lysine residue and the third lysine residue. In some embodiments, the ubiquitination at a first lysine residue, a second lysine residue, a third lysine residue, or a fourth lysine residue, or any combinations thereof comprises ubiquitination at the second lysine residue and the fourth lysine residue. In some embodiments, the ubiquitination at a first lysine residue, a second lysine residue, a third lysine residue, or a fourth lysine residue, or any combinations thereof comprises ubiquitination at the third lysine residue and the fourth lysine residue.

In some embodiments, the ubiquitination at a first lysine residue, a second lysine residue, a third lysine residue, or a fourth lysine residue, or any combinations thereof comprises ubiquitination at the first lysine residue, the second lysine residue, and the third lysine residue. In some embodiments, the ubiquitination at a first lysine residue, a second lysine residue, a third lysine residue, or a fourth lysine residue, or any combinations thereof comprises ubiquitination at the first lysine residue, the second lysine residue, and the fourth lysine residue. In some embodiments, the ubiquitination at a first lysine residue, a second lysine residue, a third lysine residue, or a fourth lysine residue, or any combinations thereof comprises ubiquitination at the first lysine residue, the third lysine residue, and the fourth lysine residue. In some embodiments, the ubiquitination at a first lysine residue, a second lysine residue, a third lysine residue, or a fourth lysine residue, or any combinations thereof comprises ubiquitination at the second lysine residue, the third lysine residue, and the fourth lysine residue.

In some embodiments, the ubiquitination at a first lysine residue, a second lysine residue, a third lysine residue, or a fourth lysine residue, or any combinations thereof comprises ubiquitination at the first lysine residue, the second lysine residue, the third lysine residue, and the fourth lysine residue.

In some embodiments, the ubiquitination at a fifth lysine residue, a sixth lysine residue, a seventh lysine residue, or an eighth lysine residue, or any combinations thereof comprises ubiquitination at the fifth lysine residue and the sixth lysine residue. In some embodiments, the ubiquitination at a fifth lysine residue, a sixth lysine residue, a seventh lysine residue, or an eighth lysine residue, or any combinations thereof comprises ubiquitination at the fifth lysine residue and the seventh lysine residue. In some embodiments, the ubiquitination at a fifth lysine residue, a sixth lysine residue, a seventh lysine residue, or an eighth lysine residue, or any combinations thereof comprises ubiquitination at the fifth lysine residue and the eighth lysine residue. In some embodiments, the ubiquitination at a fifth lysine residue, a sixth lysine residue, a seventh lysine residue, or an eighth lysine residue, or any combinations thereof comprises ubiquitination at the sixth lysine residue and the seventh lysine residue. In some embodiments, the ubiquitination at a fifth lysine residue, a sixth lysine residue, a seventh lysine residue, or an eighth lysine residue, or any combinations thereof comprises ubiquitination at the sixth lysine residue and the eighth lysine residue. In some embodiments, the ubiquitination at a fifth lysine residue, a sixth lysine residue, a seventh lysine residue, or an eighth lysine residue, or any combinations thereof comprises ubiquitination at the seventh lysine residue and the eighth lysine residue.

In some embodiments, the ubiquitination at a fifth lysine residue, a sixth lysine residue, a seventh lysine residue, or an eighth lysine residue, or any combinations thereof comprises ubiquitination at the fifth lysine residue, the sixth lysine residue, and the seventh lysine residue. In some embodiments, the ubiquitination at a fifth lysine residue, a sixth lysine residue, a seventh lysine residue, or an eighth lysine residue, or any combinations thereof comprises ubiquitination at the fifth lysine residue, the sixth lysine residue, and the eighth lysine residue. In some embodiments, the ubiquitination at a fifth lysine residue, a sixth lysine residue, a seventh lysine residue, or an eighth lysine residue, or any combinations thereof comprises ubiquitination at the fifth lysine residue, the seventh lysine residue, and the eighth lysine residue. In some embodiments, the ubiquitination at a fifth lysine residue, a sixth lysine residue, a seventh lysine residue, or an eighth lysine residue, or any combinations thereof comprises ubiquitination at the sixth lysine residue, the seventh lysine residue, and the eighth lysine residue.

In some embodiments, the ubiquitination at a fifth lysine residue, a sixth lysine residue, a seventh lysine residue, or an eighth lysine residue, or any combinations thereof comprises ubiquitination at the fifth lysine residue, the sixth lysine residue, the seventh lysine residue, and the eighth lysine residue.

In some embodiments, the first lysine residue is lysine 504 (K⁵⁰⁴). In some embodiments, the second lysine residue is lysine 595 (K⁵⁹⁵). In some embodiments, the third lysine residue is lysine 630 (K⁶³⁰). In some embodiments, the fourth lysine residue is lysine 647 (K⁶⁴⁷). In some embodiments, the fifth lysine residue is lysine 124 (K¹⁴). In some embodiments, the sixth lysine residue is lysine 307 (K³⁰⁷) In some embodiments, the seventh lysine residue is lysine 319 (K³¹⁰). In some embodiments, the eighth lysine residue is lysine 618 (K⁶¹⁸).

In some embodiments, the ubiquitination profile of the plurality of polypeptides or fragments thereof comprises ubiquitination at a first lysine residue and ubiquitination at a fifth lysine residue, wherein:
(a) the first lysine residue is lysine 504 (K⁵⁰⁴);
(b) the fifth lysine residue is lysine 124 (K¹²⁴); and
(c) the ubiquitination profile is characterized by a signal intensity ratio by mass spectrum of at least about 100:1 (e.g., at least about 110:1, at least about 120:1, at least about 130:1, at least about 140:1, at least about 150:1, at least about 160:1, at least about 170:1, at least about 180:1, at least about 190:1, at least about 200:1, at least about 210:1, at least about 220:1, at least about 230:1, at least about 240:1, or at least about 250:1) for ubiquitination at the first lysine residue to ubiquitination at the fifth lysine residue.

In some embodiments, the ubiquitination profile of the plurality of polypeptides or fragments thereof comprises ubiquitination at a first lysine residue and ubiquitination at a sixth lysine residue, wherein:
(a) the first lysine residue is lysine 504 (K⁵⁰⁴);
(b) the sixth lysine residue is lysine 307 (K³⁰⁷); and
(c) the ubiquitination profile is characterized by a signal intensity ratio by mass spectrum of at least about 1:1 (e.g., at least about 5:1, at least about 10:1, at least about 15:1, at least about 20:1, at least about 25:1, at least about 30:1, at least about 35:1, at least about 40:1, at least about 45:1, at least about 50:1, at least about 55:1, at least about 65:1, at least about 70:1, at least about 75:1, at least about 80:1, at least about 85:1, at least about 90:1, or at least about 100:1) for ubiquitination at the first lysine residue to ubiquitination at the sixth lysine residue.

In some embodiments, the ubiquitination profile of the plurality of polypeptides or fragments thereof comprises ubiquitination at a first lysine residue and ubiquitination at a seventh lysine residue, wherein:
(a) the first lysine residue is lysine 504 (K⁵⁰⁴):
(b) the seventh lysine residue is lysine 319 (K³¹⁹); and
(c) the ubiquitination profile is characterized by a signal intensity ratio by mass spectrum of at least about 1:1 (e.g., at least about 5:1, at least about 10:1, at least about 15:1, at least about 20:1, at least about 25:1, at least about 30:1, at least about 35:1, at least about 40:1, at least about 45:1, at least about 50:1, at least about 55:1, at least about 65:1, at least about 70:1, at least about 75:1, at least about 80:1, at least about 85:1, at least about 90:1, or at least about 100:1) for ubiquitination at the first lysine residue to ubiquitination at the seventh lysine residue.

In some embodiments, the ubiquitination profile of the plurality of polypeptides or fragments thereof comprises ubiquitination at a first lysine residue and ubiquitination at an eighth lysine residue, wherein:
(a) the first lysine residue is lysine 504 (K⁵⁰⁴);
(b) the eighth lysine residue is lysine 618 (K⁶¹⁸); and
(c) the ubiquitination profile is characterized by a signal intensity ratio by mass spectrum of at least about 20:1 (e.g., at least about 25:1, at least about 30:1, at least about 35:1, at least about 40:1, at least about 45:1, at least about 50:1, at least about 55:1, at least about 65:1, at least about 70:1, at least about 75:1, at least about 80:1, at least about 85:1, at least about 90:1, at least about 100:1, at least about 110:1, at least about 120:1, at least about 130:1, at least about 140:1, at least about 150:1, at least about 160:1, at least about 170:1, at least about 180:1, at least about 190:1, or at least about 200:1) for ubiquitination at the first lysine residue to ubiquitination at the eighth lysine residue.

In some embodiments, the ubiquitination profile of the plurality of polypeptides or fragments thereof comprises ubiquitination at a second lysine residue and ubiquitination at a fifth lysine residue, wherein:
(a) the second lysine residue is lysine 595 (K⁵⁹⁵);
(b) the fifth lysine residue is lysine 124 (K¹²⁴); and
(c) the ubiquitination profile is characterized by a signal intensity ratio by mass spectrum of at least about 1:1 (e.g., at least about 5:1, at least about 10:1, at least about 15:1, at least about 20:1, at least about 25:1, at least about 30:1, at least about 35:1, at least about 40:1, at least about 45:1, at least about 50:1, at least about 55:1, at least about 65:1, at least about 70:1, at least about 75:1, at least about 80:1, at least about 85:1, at least about 90:1, or at least about 100:1) for ubiquitination at the second lysine residue to ubiquitination at the fifth lysine residue.

In some embodiments, the ubiquitination profile of the plurality of polypeptides or fragments thereof comprises ubiquitination at a second lysine residue and ubiquitination at a sixth lysine residue, wherein:
(a) the second lysine residue is lysine 595 (K⁵⁹⁵);
(b) the sixth lysine residue is lysine 307 (K³⁰⁷); and
(c) the ubiquitination profile is characterized by a signal intensity ratio by mass spectrum of at least about 1:1 (e.g., at least about 5:1, at least about 10:1, at least about 15:1, at least about 20:1, at least about 25:1, at least about 30:1, at least about 35:1, at least about 40:1, at least about 45:1, at least about 50:1, at least about 55:1, at least about 65:1, at least about 70:1, at least about 75:1, at least about 80:1, at least about 85:1, at least about 90:1, or at least about 100:1) for ubiquitination at the second lysine residue to ubiquitination at the sixth lysine residue.

In some embodiments, the ubiquitination profile of the plurality of polypeptides or fragments thereof comprises ubiquitination at a second lysine residue and ubiquitination at a seventh lysine residue, wherein:
(a) the second lysine residue is lysine 595 (K⁵⁹⁵);
(b) the seventh lysine residue is lysine 319 (K³¹⁹); and
(c) the ubiquitination profile is characterized by a signal intensity ratio by mass spectrum of at least about 1:1 (e.g., at least about 5:1, at least about 10:1, at least about 15:1, at least about 20:1, at least about 25:1, at least about 30:1, at least about 35:1, at least about 40:1, at least about 45:1, at least about 50:1, at least about 55:1, at least about 65:1, at least about 70:1, at least about 75:1, at least about 80:1, at least about 85:1, at least about 90:1, or at least about 100:1) for ubiquitination at the second lysine residue to ubiquitination at the seventh lysine residue.

In some embodiments, the ubiquitination profile of the plurality of polypeptides or fragments thereof comprises ubiquitination at a first lysine residue and ubiquitination at an eighth lysine residue, wherein:
(a) the second lysine residue is lysine 595 (K⁵⁹⁵);
(b) the eighth lysine residue is lysine 618 (K⁶¹⁸); and
(c) the ubiquitination profile is characterized by a signal intensity ratio by mass spectrum of at least about 1:1 (e.g., at least about 5:1, at least about 10:1, at least about 15:1, at least about 20:1, at least about 25:1, at least about 30:1, at least about 35:1, at least about 40:1, at least about 45:1, at least about 50:1, at least about 55:1, at least about 65:1, at least about 70:1, at least about 75:1, at least about 80:1, at least about 85:1, at least about 90:1, or at least about 100:1) for ubiquitination at the second lysine residue to ubiquitination at the eighth lysine residue.

In some embodiments, the ubiquitination profile of the plurality of polypeptides or fragments thereof comprises ubiquitination at a second lysine residue and ubiquitination at a fifth lysine residue, wherein:
(a) the third lysine residue is lysine 630 (K⁶³⁰)
(b) the fifth lysine residue is lysine 124 (K¹¹⁴); and
(c) the ubiquitination profile is characterized by a signal intensity ratio by mass spectrum of at least about 20:1 (e.g., at least about 25:1, at least about 30:1, at least about 35:1, at least about 40:1, at least about 45:1, at least about 50:1, at least about 55:1, at least about 65:1, at least about 70:1, at least about 75:1, at least about 80:1, at least about 85:1, at least about 90:1, at least about 100:1, at least about 110:1, at least about 120:1, at least about 130:1, at least about 140:1, at least about 150:1, at least about 160:1, at least about 170:1, at least about 180:1, at least about 190:1, or at least about 200:1) for ubiquitination at the third lysine residue to ubiquitination at the fifth lysine residue.

In some embodiments, the ubiquitination profile of the plurality of polypeptides or fragments thereof comprises ubiquitination at a third lysine residue and ubiquitination at a sixth lysine residue, wherein:
(a) the third lysine residue is lysine 630 (K⁶³⁰)
(b) the sixth lysine residue is lysine 307 (K³⁰⁷); and
(c) the ubiquitination profile is characterized by a signal intensity ratio by mass spectrum of at least about 1:1 (e.g., at least about 5:1, at least about 10:1, at least about 15:1, at least about 20:1, at least about 25:1, at least about 30:1, at least about 35:1, at least about 40:1, at least about 45:1, at least about 50:1, at least about 55:1, at least about 65:1, at least about 70:1, at least about 75:1, at least about 80:1, at least about 85:1, at least about 90:1, or at least about 100:1) for ubiquitination at the third lysine residue to ubiquitination at the sixth lysine residue.

In some embodiments, the ubiquitination profile of the plurality of polypeptides or fragments thereof comprises ubiquitination at a second lysine residue and ubiquitination at a seventh lysine residue, wherein:
(a) the third lysine residue is lysine 630 (K⁶³⁰):
(b) the seventh lysine residue is lysine 319 (K³¹⁹); and
(c) the ubiquitination profile is characterized by a signal intensity ratio by mass spectrum of at least about 1:1 (e.g., at least about 5:1, at least about 10:1, at least about 15:1, at least about 20:1, at least about 25:1, at least about 30:1, at least about 35:1, at least about 40:1, at least about 45:1, at least about 50:1, at least about 55:1, at least about 65:1, at least about 70:1, at least about 75:1, at least about 80:1, at least about 85:1, at least about 90:1, or at least about 100:1) for ubiquitination at the second lysine residue to ubiquitination at the seventh lysine residue.

In some embodiments, the ubiquitination profile of the plurality of polypeptides or fragments thereof comprises ubiquitination at a third lysine residue and ubiquitination at an eighth lysine residue, wherein:
(a) the third lysine residue is lysine 630 (K⁶³⁰):
(b) the eighth lysine residue is lysine 618 (K⁶¹⁸); and
(c) the ubiquitination profile is characterized by a signal intensity ratio by mass spectrum of at least about 20:1 (e.g., at least about 25:1, at least about 30:1, at least about 35:1, at least about 40:1, at least about 45:1, at least about 50:1, at least about 55:1, at least about 65:1, at least about 70:1, at least about 75:1, at least about 80:1, at least about 85:1, at least about 90:1, at least about 100:1, at least about 110:1, at least about 120:1, at least about 130:1, at least about 140:1, at least about 150:1, at least about 160:1, at least about 170:1, at least about 180:1, at least about 190:1, or at least about 200:1 (e.g., about 20:1 to about 150:1, about 20:1 to about 100:1, about 20:1 to about 50:1, about 40:1 to about 180:1, about 40:1 to about 160:1, about 40:1 to about 140:1, about 40:1 to about 120:1, or about 60:1 to about 120:1) for ubiquitination at the third lysine residue to ubiquitination at the eighth lysine residue.

In some embodiments, the ubiquitination profile of the plurality of polypeptides or fragments thereof comprises ubiquitination at a fourth lysine residue and ubiquitination at a fifth lysine residue, wherein:
(a) the fourth lysine residue is lysine 647 (K⁶⁴⁷);
(b) the fifth lysine residue is lysine 124 (K²⁴); and
(c) the ubiquitination profile is characterized by a signal intensity ratio by mass spectrum of at least about 1:1 (e.g., at least about 5:1, at least about 10:1, at least about 15:1, at least about 20:1, at least about 25:1, at least about 30:1, at least about 35:1, at least about 40:1, at least about 45:1, at least about 50:1, at least about 55:1, at least about 65:1, at least about 70:1, at least about 75:1, at least about 80:1, at least about 85:1, at least about 90:1, or at least about 100:1) for ubiquitination at the fourth lysine residue to ubiquitination at the fifth lysine residue.

In some embodiments, the ubiquitination profile of the plurality of polypeptides or fragments thereof comprises ubiquitination at a fourth lysine residue and ubiquitination at a sixth lysine residue, wherein:
(a) the fourth lysine residue is lysine 647 (K⁶⁴⁷);
(b) the sixth lysine residue is lysine 307 (K³⁰⁷); and
(c) the ubiquitination profile is characterized by a signal intensity ratio by mass spectrum of at least about 1:1 (e.g., at least about 5:1, at least about 10:1, at least about 15:1, at least about 20:1, at least about 25:1, at least about 30:1, at least about 35:1, at least about 40:1, at least about 45:1, at least about 50:1, at least about 55:1, at least about 65:1, at least about 70:1, at least about 75:1, at least about 80:1, at least about 85:1, at least about 90:1, or at least about 100:1) for ubiquitination at the fourth lysine residue to ubiquitination at the sixth lysine residue.

In some embodiments, the ubiquitination profile of the plurality of polypeptides or fragments thereof comprises ubiquitination at a fourth lysine residue and ubiquitination at a seventh lysine residue, wherein:
(a) the fourth lysine residue is lysine 647 (K⁶⁴⁷);
(b) the seventh lysine residue is lysine 319 (K³¹⁹); and
(c) the ubiquitination profile is characterized by a signal intensity ratio by mass spectrum of at least about 1:1 (e.g., at least about 5:1, at least about 10:1, at least about 15:1, at least about 20:1, at least about 25:1, at least about 30:1, at least about 35:1, at least about 40:1, at least about 45:1, at least about 50:1, at least about 55:1, at least about 65:1, at least about 70:1, at least about 75:1, at least about 80:1, at least about 85:1, at least about 90:1, or at least about 100:1) for ubiquitination at the fourth lysine residue to ubiquitination at the seventh lysine residue.

In some embodiments, the ubiquitination profile of the plurality of polypeptides or fragments thereof comprises ubiquitination at a fourth lysine residue and ubiquitination at an eighth lysine residue, wherein:
(a) the fourth lysine residue is lysine 647 (K⁶⁴⁷);
(b) the eighth lysine residue is lysine 618 (K⁶¹⁸); and
(c) the ubiquitination profile is characterized by a signal intensity ratio by mass spectrum of at least about 1:1 (e.g., at least about 5:1, at least about 10:1, at least about 15:1, at least about 20:1, at least about 25:1, at least about 30:1, at least about 35:1, at least about 40:1, at least about 45:1, at least about 50:1, at least about 55:1, at least about 65:1, at least about 70:1, at least about 75:1, at least about 80:1, at least about 85:1, at least about 90:1, or at least about 100:1) for ubiquitination at the fourth lysine residue to ubiquitination at the eighth lysine residue.

In some embodiments, the ubiquitination profile of the plurality of polypeptides or fragments thereof comprises ubiquitination at a first lysine residue, ubiquitination at a third lysine residue, ubiquitination at a fifth lysine residue, and ubiquitination at an eighth lysine residue, wherein:
(a) the first lysine residue is lysine 504 (K⁵⁰⁴):
(b) the third lysine residue is lysine 630 (K⁶³⁰):
(c) the fifth lysine residue is lysine 124 (K¹²⁴);
(d) the eighth lysine residue is lysine 618 (K⁶¹⁸); and
(e) the ubiquitination profile is characterized by a signal intensity ratio by mass spectrum of at least about 5:1 (e.g., at least about 6:1, at least about 7:1, at least about 8:1, at least about 9:1, at least about 10:1, at least about 11:1, at least about 12:1, at least about 13:1, at least about 14:1, or at least about 15:1) for the sum of ubiquitination at the first lysine residue and ubiquitination at the third lysine residue to the sum of ubiquitination at the fifth lysine residue and ubiquitination at the eighth lysine residue.

In some embodiments, the ubiquitination profile of the plurality of polypeptides or fragments thereof comprises ubiquitination at a first lysine residue, ubiquitination at a fourth lysine residue, ubiquitination at a fifth lysine residue, and ubiquitination at an eighth lysine residue, wherein:
(a) the first lysine residue is lysine 504 (K⁵⁰⁴);
(b) the fourth lysine residue is lysine 647 (K⁶⁴⁷);
(c) the fifth lysine residue is lysine 124 (K¹²⁴);
(d) the eighth lysine residue is lysine 618 (K⁶¹⁸); and
(e) the ubiquitination profile is characterized by a signal intensity ratio by mass spectrum of at least about 5:1 (e.g., at least about 6:1, at least about 7:1, at least about 8:1, at least about 9:1, at least about 10:1, at least about 11:1, at least about 12:1, at least about 13:1, at least about 14:1, or at least about 15:1) for the sum of ubiquitination at the first lysine residue and ubiquitination at the fourth lysine residue to the sum of ubiquitination at the fifth lysine residue and ubiquitination at the eighth lysine residue.

In some embodiments, the ubiquitination profile of the plurality of polypeptides or fragments thereof comprises ubiquitination at a first lysine residue, ubiquitination at a third lysine residue, ubiquitination at a fourth lysine residue, ubiquitination at a fifth lysine residue, ubiquitination at a sixth lysine residue, and ubiquitination at an eighth lysine residue, wherein:
(a) the first lysine residue is lysine 504 (K⁵⁰⁴);
(b) the third lysine residue is lysine 630 (K⁶³⁰):
(c) the fourth lysine residue is lysine 647 (K⁶⁴⁷);
(d) the fifth lysine residue is lysine 124 (K¹²⁴):
(e) the sixth lysine residue is lysine 307 (K³⁰⁷);
(f) the eighth lysine residue is lysine 618 (K⁶¹⁸); and
(g) the ubiquitination profile is characterized by a signal intensity ratio by mass spectrum of at least about 5:1 (e.g., at least about 6:1, at least about 7:1, at least about 8:1, at least about 9:1, at least about 10:1, at least about 11:1, at least about 12:1, at least about 13:1, at least about 14:1, or at least about 15:1) for the sum of ubiquitination at the first lysine residue, ubiquitination at the third lysine residue, and ubiquitination at the fourth lysine residue to the sum of ubiquitination at the fifth lysine residue, ubiquitination at the sixth lysine residue, and ubiquitination at the eighth lysine residue.

In some embodiments, the ubiquitination profile of the plurality of polypeptides or fragments thereof comprises ubiquitination at a first lysine residue, ubiquitination at a third lysine residue, ubiquitination at a fourth lysine residue, ubiquitination at a fifth lysine residue, ubiquitination at a sixth lysine residue, and ubiquitination at an eighth lysine residue, wherein:
(a) the first lysine residue is lysine 504 (K⁵⁰⁴):
(b) the third lysine residue is lysine 630 (K⁶³⁰);
(c) the fourth lysine residue is lysine 647 (K⁶⁴⁷);
(d) the fifth lysine residue is lysine 124 (K¹²⁴):
(e) the sixth lysine residue is lysine 307 (K³⁰⁷);
(f) the eighth lysine residue is lysine 618 (K⁶¹⁸); and
(g) the ubiquitination profile is characterized by mass spectrum and indicates that the sum of ubiquitination at the first lysine residue, ubiquitination at the third lysine residue, and ubiquitination at the fourth lysine residue is at least about 50% (e.g., at least about 55%, at least about 60%, at least about 65%, at least about 70%, or at least about 75%) of the total ubiquitination of the plurality of polypeptides or fragments thereof.

In any of the embodiments described herein, the ubiquitination profile is characterized by a signal intensity ratio by mass spectrum of at least about 1:1 (e.g., at least about 5:1, at least about 10:1, at least about 15:1, at least about 20:1, at least about 25:1, at least about 30:1, at least about 35:1, at least about 40:1, at least about 45:1, at least about 50:1, at least about 55:1, at least about 65:1, at least about 70:1, at least about 75:1, at least about 80:1, at least about 85:1, at least about 90:1, or at least about 100:1 (e.g., about 1:1 to about 100:1, about 10:1 to about 90:1, about 20:1 to about 80:1, about 30:1 to about 70:1, about 40:1 to about 60:1, about 1:1 to about 10:1, about 1:1 to about 20:1, about 1:1 to about 50:1, about 90:1 to about 100:1, about 80:1 to about 100:1, about 50:1 to about 100:1 (e.g., about 1:1, about 2:1, about 3:1, about 4:1, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, about 20:1, about 21:1, about 22:1, about 23:1, about 24:1, about 25:1, about 26:1, about 27:1, about 28:1, about 29:1, about 30:1, about 31:1, about 32:1, about 33:1, about 34:1, about 35:1, about 36:1, about 37:1, about 38:1, about 39:1, about 40:1, about 41:1, about 42:1, about 43:1, about 44:1, about 45:1, about 46:1, about 47:1, about 48:1, about 49:1, about 50:1, about 51:1, about 52:1, about 53:1, about 54:1, about 55:1, about 56:1, about 57:1, about 58:1, about 59:1, about 60:1, about 61:1, about 62:1, about 63:1, about 64:1, about 65:1, about 66:1, about 67:1, about 68:1, about 69:1, about 70:1, about 71:1, about 72:1, about 73:1, about 74:1, about 75:1, about 76:1, about 77:1, about 78:1, about 79:1, about 80:1, about 81:1, about 82:1, about 83:1, about 84:1, about 85:1, about 86:1, about 87:1, about 88:1, about 89:1, about 90:1, about 91:1, about 92:1, about 93:1, about 94:1, about 95:1, about 96:1, about 97:1, about 98:1, about 99:1, or about 100:1))) for (i) ubiquitination at a first lysine residue, a second lysine residue, a third lysine residue, or a fourth lysine residue, or any combinations thereof; to (ii) ubiquitination at a fifth lysine residue, a sixth lysine residue, a seventh lysine residue, or an eighth lysine residue, or any combinations thereof.

In any of the embodiments described herein, the ubiquitination profile is characterized by a signal intensity ratio by mass spectrum of at least about 20:1 (e.g., at least about 25:1, at least about 30:1, at least about 35:1, at least about 40:1, at least about 45:1, at least about 50:1, at least about 55:1, at least about 65:1, at least about 70:1, at least about 75:1, at least about 80:1, at least about 85:1, at least about 90:1, at least about 100:1, at least about 110:1, at least about 120:1, at least about 130:1, at least about 140:1, at least about 150:1, at least about 160:1, at least about 170:1, at least about 180:1, at least about 190:1, or at least about 200:1 (e.g., about 20:1 to about 150:1, about 20:1 to about 100:1, about 20:1 to about 50:1, about 40:1 to about 180:1, about 40:1 to about 160:1, about 40:1 to about 140:1, about 40:1 to about 120:1, or about 60:1 to about 120:1, (e.g., about 20:1, about 21:1, about 22:1, about 23:1, about 24:1, about 25:1, about 26:1, about 27:1, about 28:1, about 29:1, about 30:1, about 31:1, about 32:1, about 33:1, about 34:1, about 35:1, about 36:1, about 37:1, about 38:1, about 39:1, about 40:1, about 41:1, about 42:1, about 43:1, about 44:1, about 45:1, about 46:1, about 47:1, about 48:1, about 49:1, about 50:1, about 51:1, about 52:1, about 53:1, about 54:1, about 55:1, about 56:1, about 57:1, about 58:1, about 59:1, about 60:1, about 61:1, about 62:1, about 63:1, about 64:1, about 65:1, about 66:1, about 67:1, about 68:1, about 69:1, about 70:1, about 71:1, about 72:1, about 73:1, about 74:1, about 75:1, about 76:1, about 77:1, about 78:1, about 79:1, about 80:1, about 81:1, about 82:1, about 83:1, about 84:1, about 85:1, about 86:1, about 87:1, about 88:1, about 89:1, about 90:1, about 91:1, about 92:1, about 93:1, about 94:1, about 95:1, about 96:1, about 97:1, about 98:1, about 99:1, about 100:1, about 101:1, about 102:1, about 103:1, about 104:1, about 105:1, about 106:1, about 107:1, about 108:1, about 109:1, about 110:1, about 111:1, about 112:1, about 113:1, about 114:1, about 115:1, about 116:1, about 117:1, about 118:1, about 119:1, about 120:1, about 121:1, about 122:1, about 123:1, about 124:1, about 125:1, about 126:1, about 127:1, about 128:1, about 129:1, about 130:1, about 131:1, about 132:1, about 133:1, about 134:1, about 135:1, about 136:1, about 137:1, about 138:1, about 139:1, about 140:1, about 141:1, about 142:1, about 143:1, about 144:1, about 145:1, about 146:1, about 147:1, about 148:1, about 149:1, about 150:1, about 151:1, about 152:1, about 153:1, about 154:1, about 155:1, about 156:1, about 157:1, about 158:1, about 159:1, about 160:1, about 161:1, about 162:1, about 163:1, about 164:1, about 165:1, about 166:1, about 167:1, about 168:1, about 169:1, about 170:1, about 171:1, about 172:1, about 173:1, about 174:1, about 175:1, about 176:1, about 177:1, about 178:1, about 179:1, about 180:1, about 181:1, about 182:1, about 183:1, about 184:1, about 185:1, about 186:1, about 187:1, about 188:1, about 189:1, about 190:1, about 191:1, about 192:1, about 193:1, about 194:1, about 195:1, about 196:1, about 197:1, about 198:1, about 199:1, or about 200:1))) for (i) ubiquitination at a first lysine residue, a second lysine residue, a third lysine residue, or a fourth lysine residue, or any combinations thereof; to (ii) ubiquitination at a fifth lysine residue, a sixth lysine residue, a seventh lysine residue, or an eighth lysine residue, or any combinations thereof.

In any of the embodiments described herein, the ubiquitination profile is characterized by a signal intensity ratio by mass spectrum of at least about 100:1 (e.g., at least about 110:1, at least about 120:1, at least about 130:1, at least about 140:1, at least about 150:1, at least about 160:1, at least about 170:1, at least about 180:1, at least about 190:1, at least about 200:1, at least about 210:1, at least about 220:1, at least about 230:1, at least about 240:1, or at least about 250:1 (e.g., about 100:1 to about 250:1, about 125:1 to about 225:1, or about 150:1 to about 200:1 (e.g., about 100:1, about 101:1, about 102:1, about 103:1, about 104:1, about 105:1, about 106:1, about 107:1, about 108:1, about 109:1, about 110:1, about 111:1, about 112:1, about 113:1, about 114:1, about 115:1, about 116:1, about 117:1, about 118:1, about 119:1, about 120:1, about 121:1, about 122:1, about 123:1, about 124:1, about 125:1, about 126:1, about 127:1, about 128:1, about 129:1, about 130:1, about 131:1, about 132:1, about 133:1, about 134:1, about 135:1, about 136:1, about 137:1, about 138:1, about 139:1, about 140:1, about 141:1, about 142:1, about 143:1, about 144:1, about 145:1, about 146:1, about 147:1, about 148:1, about 149:1, about 150:1, about 151:1, about 152:1, about 153:1, about 154:1, about 155:1, about 156:1, about 157:1, about 158:1, about 159:1, about 160:1, about 161:1, about 162:1, about 163:1, about 164:1, about 165:1, about 166:1, about 167:1, about 168:1, about 169:1, about 170:1, about 171:1, about 172:1, about 173:1, about 174:1, about 175:1, about 176:1, about 177:1, about 178:1, about 179:1, about 180:1, about 181:1, about 182:1, about 183:1, about 184:1, about 185:1, about 186:1, about 187:1, about 188:1, about 189:1, about 190:1, about 191:1, about 192:1, about 193:1, about 194:1, about 195:1, about 196:1, about 197:1, about 198:1, about 199:1, about 200:1, about 201:1, about 202:1, about 203:1, about 204:1, about 205:1, about 206:1, about 207:1, about 208:1, about 209:1, about 210:1, about 211:1, about 212:1, about 213:1, about 214:1, about 215:1, about 216:1, about 217:1, about 218:1, about 219:1, about 220:1, about 221:1, about 222:1, about 223:1, about 224:1, about 225:1, about 226:1, about 227:1, about 228:1, about 229:1, about 230:1, about 231:1, about 232:1, about 233:1, about 234:1, about 235:1, about 236:1, about 237:1, about 238:1, about 239:1, about 240:1, about 241:1, about 242:1, about 243:1, about 244:1, about 245:1, about 246:1, about 247:1, about 248:1, about 249:1, or about 250:1)) for (i) ubiquitination at a first lysine residue, a second lysine residue, a third lysine residue, or a fourth lysine residue, or any combinations thereof; to (ii) ubiquitination at a fifth lysine residue, a sixth lysine residue, a seventh lysine residue, or an eighth lysine residue, or any combinations thereof.

In some embodiments, the ubiquitination profile of the plurality of polypeptides or fragments thereof comprises ubiquitination of any combination of the embodiments described herein. In some embodiments, the ubiquitination profile of the plurality of polypeptides or fragments thereof is characterized by any combination of signal intensity ratios by mass spectrum of the embodiments described herein.

Another aspect of the present disclosure relates to a method of treating a STAT6-mediated disorder, disease, or condition in a subject in need thereof, wherein the method comprises administering to said subject a compound disclosed herein, or a pharmaceutical acceptable salt thereof; and wherein the method results in the subject having a reduced STAT6 level relative to the subject prior to administering the compound, or a pharmaceutical acceptable salt thereof.

Another aspect of the present disclosure relates to a method of treating a STAT6-mcdiatcd disorder, disease, or condition in a subject in need thereof, wherein the method comprises administering to said subject a compound disclosed herein, or a pharmaceutical acceptable salt thereof; and wherein the method results in the subject having a reduced STAT6 level relative to an untreated subject with the same STAT6-mediated disorder, disease, or condition.

In some embodiments, the STAT6 is from a sample. In some embodiments, a sample comprises a biological sample. In some embodiments, the biological sample is taken from the subject. In some embodiments, the sample comprises a lysate. In some embodiments, the sample comprises a peripheral blood mononuclear cell (PBMC).

Another aspect of the present disclosure relates to a method of degrading STAT6 in a subject, wherein the method comprises administering to said subject a compound disclosed herein, or a pharmaceutical acceptable salt thereof; and wherein the method results in the subject having a reduced STAT6 level relative to the subject prior to administering the compound, or a pharmaceutical acceptable salt thereof.

Another aspect of the present disclosure relates to a method of degrading STAT6 in a subject, wherein the method comprises administering to said subject a compound disclosed herein, or a pharmaceutical acceptable salt thereof; and wherein the method results in the subject having a reduced STAT6 level relative to an untreated subject with the same STAT6-mediated disorder, disease, or condition.

In some embodiments, the STAT6 is from a sample. In some embodiments, the sample comprises a biological sample. In some embodiments, the biological sample is taken from the subject. In some embodiments, the sample comprises a lysate. In some embodiments, the sample comprises a peripheral blood mononuclear cell (PBMC). In some embodiments, the STAT6 is a polypeptide disclosed herein. In some embodiments, the STAT6 is a polypeptide of a plurality of polypeptides disclosed herein.

### 4. General Methods of Providing the Present Compounds

The compounds of this invention may be prepared or isolated in general by synthetic and/or semi-synthetic methods known to those skilled in the art for analogous compounds and by methods described in detail in the Examples, herein.

In certain embodiments, compounds of the present invention are generally prepared according to any one of the schemes set forth below:

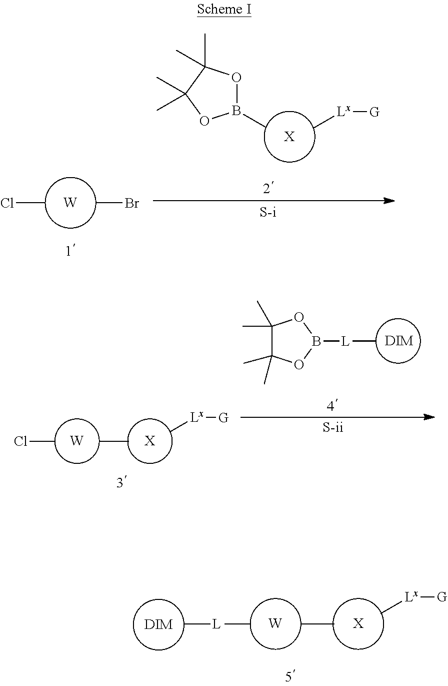

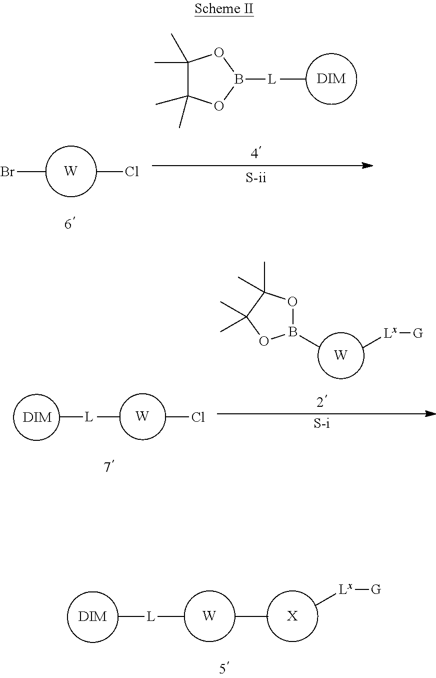

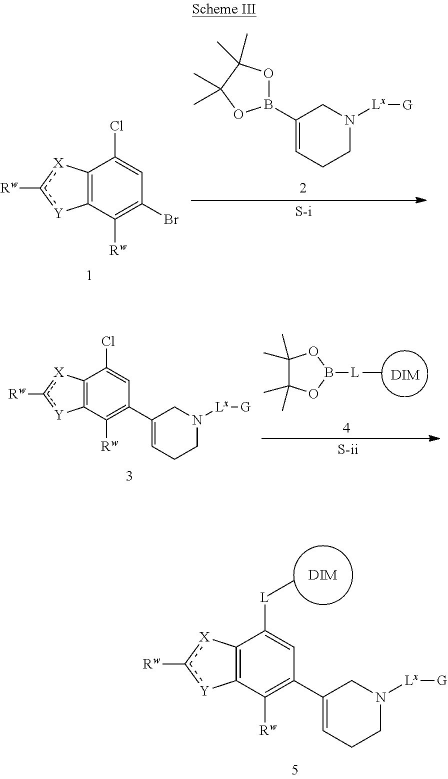

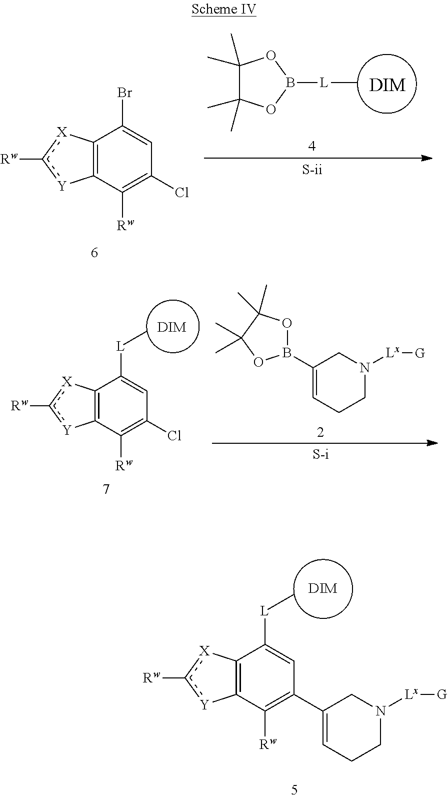

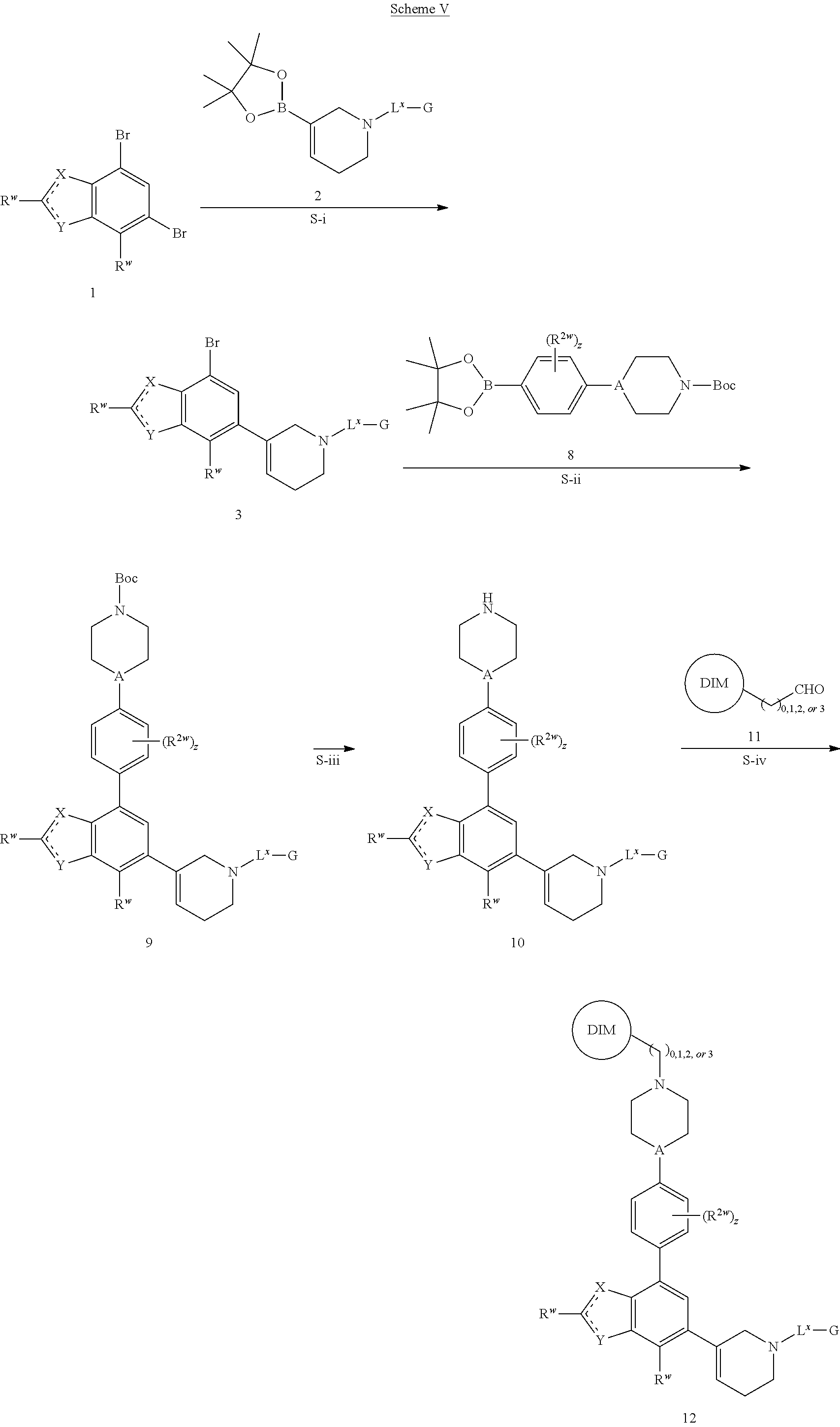

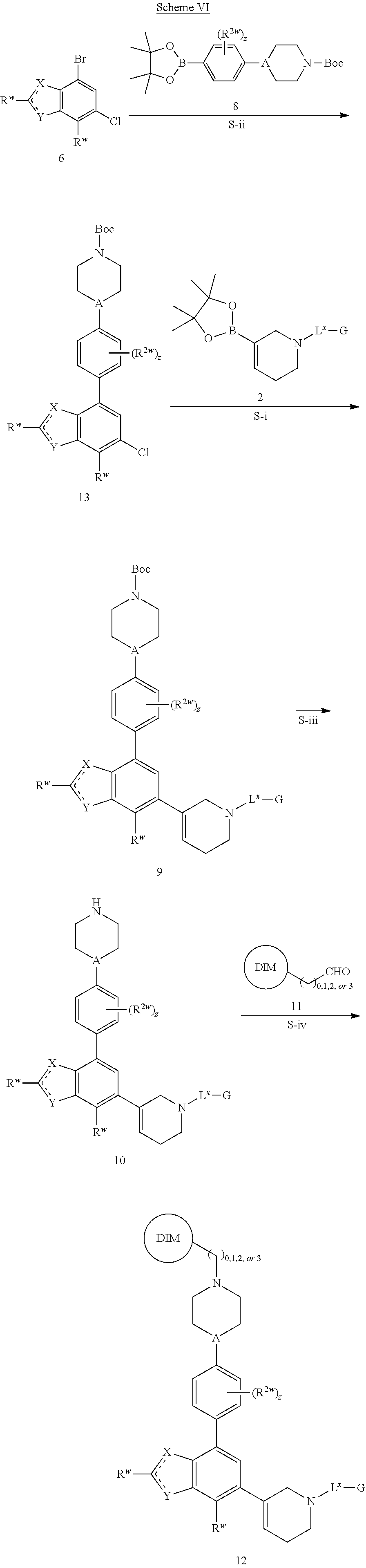

In the schemes above, each of Ring W, Ring X, X, Y, R^{w}, R^{2w}, L^{x}, G, and z is as defined above and in classes and subclasses as described herein.

In one aspect, the present invention provides methods for preparing certain compounds of formula I according to the steps depicted in Scheme I-VI above. In some embodiments, step S-i comprises contacting a compound of formula 1, 1′. 7, 7′, or 13 with a compound of formula 2 or 2′ in the presence of a palladium catalyst and a base in a solvent. In some embodiments, the palladium catalyst includes a phosphine ligand (e.g., dppf). In some embodiments, the base is a carbonate (e.g., K₂CO₃ or Cs₂CO₃) or a phosphate (e.g., K₃PO₄). In some embodiments, the solvent is an aqueous mixture including a polar aprotic solvent (e.g., dioxane). In some embodiments, step S-i comprises the compounds, reagents, and conditions described in the below Examples section.

In some embodiments, step S-ii comprises contacting a compound of formula 3, 3′, 6 or 6′ with a compound of formula 4 or 4′ in the presence of a palladium catalyst and a base in a solvent. In some embodiments, the palladium catalyst includes a phosphine ligand (e.g., Xphos). In some embodiments, the base is a carbonate (e.g., K₂CO₃ or Cs₂CO₃) or a phosphate (e.g., K₃PO₄). In some embodiments, the solvent is an aqueous mixture including a polar aprotic solvent (e.g., dioxane). In some embodiments, step S-ii comprises the compounds, reagents, and conditions described in the below Examples section.

In some embodiments, step S-iii comprises contacting a compound of formula 9 with an acid in a solvent. In some embodiments, the acid is an inorganic base (e.g., HCl) or organic base (e.g., TFA). In some embodiments, the solvent is a polar aprotic solvent (e.g., DCM). In some embodiments, step S-iii comprises the compounds, reagents, and conditions described in the below Examples section.

In some embodiments, step S-iv comprises contacting a compound of formula 10 with a compound of formula 11 in the presence of an reducing agent in a solvent. In some embodiments, the reducing agent is a borohydride compound (e.g., sodium cyanoborohydride or sodium triacetoxyborohydride). In some embodiments, the solvent is a polar aprotic solvent (e.g., DMSO). In some embodiments, step S-iv comprises the compounds, reagents, and conditions described in the below Examples section.

### 5. Uses, Formulation and Administration

### Pharmaceutically Acceptable Compositions

According to another embodiment, the invention provides a composition comprising a compound of this invention or a pharmaceutically acceptable derivative thereof and a pharmaceutically acceptable carrier, adjuvant, or vehicle. The amount of compound in compositions of this invention is such that is effective to measurably degrade and/or inhibit STAT6 protein, or a mutant thereof, in a biological sample or in a patient. In certain embodiments, the amount of compound in compositions of this invention is such that is effective to measurably degrade and/or inhibit STAT6 protein, or a mutant thereof, in a biological sample or in a patient. In certain embodiments, a composition of this invention is formulated for administration to a patient in need of such composition. In some embodiments, a composition of this invention is formulated for oral administration to a patient.

The term “patient” as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this invention that, upon administration to a recipient, is capable of providing, either directly or indirectly, a compound of this invention or an inhibitorily or degratorily active metabolite or residue thereof.

As used herein, the term “inhibitory active metabolite or residue thereof” means that a metabolite or residue thereof is also an inhibitor of STAT6 protein, or a mutant thereof.

As used herein, the term “degradatory active metabolite or residue thereof” means that a metabolite or residue thereof is also a degrader of STAT6 protein, or a mutant thereof.

In certain embodiments, a provided compound is administered as a prodrug.

Compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic. pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of compounds of the present invention that may be combined with the carrier materials to produce a composition in a single dosage form will vary depending upon the host treated, the particular mode of administration. Preferably, provided compositions should be formulated so that a dosage of between 0.01-100 mg/kg body weight/day of the compound can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

### Uses of Compounds and Pharmaceutically Acceptable Compositions

Compounds and compositions described herein are generally useful for the degradation and/or inhibition of STAT6 protein activity.

According to one embodiment, the invention relates to a method of inhibiting or degrading STAT6 or a mutant thereof, activity in a biological sample comprising the step of contacting said biological sample with a compound of this invention, or a composition comprising said compound.

The term “biological sample”, as used herein, includes, without limitation, cell cultures or extracts thereof, biopsied material obtained from a mammal or extracts thereof; and blood, saliva, urine, feces, semen, tears, or other body fluids or extracts thereof. In some embodiments, the STAT6 is from a biological sample. In some embodiments, the biological sample is taken from a subject.

Inhibition and/or degradation of STAT6, or a mutant thereof, activity in a biological sample is useful for a variety of purposes that are known to one of skill in the art. Examples of such purposes include, but are not limited to, blood transfusion, organ-transplantation, biological specimen storage, and biological assays.

According to another embodiment, the invention relates to a method of degrading and/or inhibiting STAT6, or a mutant thereof, activity in a patient comprising the step of administering to said patient a compound of the present invention, or a composition comprising said compound. In other embodiments, the present invention provides a method for treating a disorder mediated by STAT6 or a mutant thereof, in a patient in need thereof, comprising the step of administering to said patient a compound according to the present invention or pharmaceutically acceptable composition thereof. Such disorders are described in detail herein.

In some embodiments, the invention described herein provides a method of degrading STAT6 in a subject, wherein the method comprises administering to said subject a compound described herein, or a pharmaceutical acceptable salt thereof; and wherein the method results in the subject having a reduced STAT6 level relative to the subject prior to administering the compound described herein, or a pharmaceutical acceptable salt thereof. In some embodiments, the STAT6 is from a biological sample. In some embodiments, the biological sample is taken from the subject. In some embodiments, the STAT6 is the polypeptide described herein.

In some embodiments, the invention described herein provides a method of degrading STAT6 in a subject, wherein the method comprises administering to said subject a compound described herein, or a pharmaceutical acceptable salt thereof; and wherein the method results in the subject having a reduced STAT6 level relative to an untreated subject with the same STAT6-mediated disorder, disease, or condition. In some embodiments, the STAT6 is from a biological sample. In some embodiments, the biological sample is taken from the subject. In some embodiments, the STAT6 is the polypeptide described herein.

In some embodiments, the invention described herein provides a method of treating a STAT6-mediated disorder, disease, or condition in a subject in need thereof, wherein the method comprises administering to said subject a compound described herein, or a pharmaceutical acceptable salt thereof; and wherein the method results in the subject having a reduced STAT6 level relative to the subject prior to administering the compound described herein, or a pharmaceutical acceptable salt thereof. In some embodiments, the STAT6 is from a biological sample. In some embodiments, the biological sample is taken from the subject. In some embodiments, the STAT6 is the polypeptide described herein.

In some embodiments, described herein is a method of treating a STAT6-mediated disorder, disease, or condition in a subject in need thereof, wherein the method comprises administering to said subject a compound described herein, or a pharmaceutical acceptable salt thereof, and wherein the method results in the subject having a reduced STAT6 level relative to an untreated subject with the same STAT6-meditated disorder, disease, or condition. In some embodiments, the STAT6 is from a biological sample. In some embodiments, the biological sample is taken from the subject. In some embodiments, the STAT6 is the polypeptide described herein.

The activity of a compound utilized in this invention as a degrader and/or inhibitor of STAT6 or a mutant thereof, may be assayed in vitro, in vivo or in a cell line. In vitro assays include assays that determine inhibition of either the activity and/or the subsequent functional consequences of activated STAT6 protein or a mutant thereof. Alternate in vitro assays quantitate the ability of the inhibitor to bind to STAT6 protein. Inhibitor binding may be measured by radiolabeling the inhibitor prior to binding, isolating the inhibitor/STAT6 complex and determining the amount of radiolabel bound. Alternatively, inhibitor binding may be determined by running a competition experiment where new inhibitors are incubated with STAT6 protein bound to known radioligands. Detailed conditions for assaying a compound utilized in this invention as a degrader and/or inhibitor of STAT proteins, or a mutant thereof, are set forth in the Examples below.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

Provided compounds are degraders and/or inhibitors of STAT6 protein and are therefore useful for treating one or more disorders associated with activity of STAT6 protein. Thus, in certain embodiments, the present invention provides a method for treating a STAT6-mediated disorder comprising the step of administering to a patient in need thereof a compound of the present invention, or pharmaceutically acceptable composition thereof.

As used herein, the term “STAT6-mediated” disorders, diseases, and/or conditions as used herein means any disease or other deleterious condition in which STAT6 or a mutant thereof, are known to play a role. Accordingly, another embodiment of the present invention relates to treating or lessening the severity of one or more diseases in which STAT6 or a mutant thereof, are known to play a role.

STAT6 functions as a transcription factor to induce gene expression and plays an important role in the IL-4/IL-13 signaling pathway and thus is critical in IL-4/IL-13 mediated biological responses including in human malignancies (e.g., Patel, B. K. R., et al. “Localization of the human stat6 gene to chromosome 12q13. 3-q14. 1, a region implicated in multiple solid tumors.” Genomics 52.2 (1998): 192-200). The STAT6-mediated signaling pathway has been shown to be required for the development of T-helper ty pe 2 (Th2) cells and Th2 immune response and plays a critical role in Th2 lung inflammatory responses including clearance of parasitic infections and in the pathogenesis of asthma (e.g., Walford, H. H. and Doherty, T. A. “STAT6 and lung inflammation.” Jak-stat 2.4 (2013): e25301). It has been found that STAT6 induces the expression of BCL2L1/BCL-X(L), which is responsible for the anti-apoptotic activity of IL-4 and is shown to play a prominent role in adaptive immunity such as providing innate immune signaling in response to virus infection (e.g., Chen, H., et al. “Activation of STAT6 by STING is critical for antiviral innate immunity.” Cell 147.2 (2011): 436-446). Knockout studies in mice have suggested the role STAT6 in differentiation of T helper 2 (Th2), expression of cell surface markers, and class switch of immunoglobulins. Activation of STAT6 signaling pathway is necessary in tumor-associated macrophages (TAMs) and is implicated in the treatment of cancers and atherosclerosis (e.g., Binnemars-Postma, K., et al. “Targeting the Stat6 pathway in tumor-associated macrophages reduces tumor growth and metastatic niche formation in breast cancer.” The FASEB Journal 32.2 (2018): 969-978; Gong, M., et al. “STAT6 upregulation promotes M2 macrophage polarization to suppress atherosclerosis.” Medical science monitor basic research 23 (2017): 240). STAT6 protein also regulates other transcription factor as Gata3, which is important regulator of Th2 differentiation. STAT6 is also required for the development of IL-9-secreting T cells. STAT6 is also involved in IL4 signaling in B cells, and STAT6 determines the levels of CD20 on the surface of normal and malignant B lymphocytes (e.g., Sandova, V., et al. “IL4-STAT6 signaling induces CD20 in chronic lymphocytic leukemia and this axis is repressed by PI3Kδ inhibitor idelalisib.” haematologica 106.11 (2021): 2995).

In some embodiments, biomarkers associated with the IL-4/13 pathway include IgE. Thymus and activation regulated chemokine (TARC), CD23, periostin, and eisinophils. TARC is a serum TH2 biomarker and chemoattractant for TH2 cell. CD23 is a B cell activation marker and correlates with IgE class switch. Periostin is a serum TH2 biomarker and ECM protein associated with tissue remodeling in atopic diseases.

In some embodiments, treatment with provided a compound results in lesser IL-4 induced TARC release compared to a reference or standard level. In some embodiments, treatment with provided a compound results in lesser IL-13 induced CD23 expression compared to a reference or standard level. In some embodiments, treatment with provided a compound results in lesser IL-13 induced periostin release compared to a reference or standard level.

In some embodiments, treatment with provided a compound inhibits IL-4 induced TARC release. In some embodiments, treatment with provided a compound inhibits IL-13 induced CD23 expression. ISE, treatment with provided a compound inhibits IL-13 induced periostin release.

In some embodiments, the present invention provides a method for treating one or more disorders, diseases, and/or conditions wherein the disorder, disease, or condition is a cancer, a neurodegenerative disorder, a viral disease, an autoimmune disease, an inflammatory disorder, a hereditary disorder, a hormone-related disease, a metabolic disorder, conditions associated with organ transplantation, immunodeficiency disorders, a destructive or overgrowing bone disorder, a proliferative disorder, an infectious disease, a condition associated with cell death, thrombin-induced platelet aggregation, liver disease, pathologic immune conditions involving T cell activation, a cardiovascular disorder, or a CNS disorder.

Diseases and conditions treatable according to the methods of this invention include, but are not limited to, cancer, cardiovascular disease, viral disease, autoimmune diseases, autoinflammatory syndromes, atherosclerosis, psoriasis, allergic disorders, inflammatory bowel disease, inflammation, acute and chronic gout and gouty arthritis, neurological disorders, metabolic syndrome, immunodeficiency disorders such as AIDS and HIV, destructive bone disorders, osteoarthritis, proliferative disorders, infectious diseases, conditions associated with cell death, pathologic immune conditions involving T cell activation, and CNS disorders in a patient. In one embodiment, a human patient is treated with a compound of the current invention and a pharmaceutically acceptable carrier, adjuvant, or vehicle, wherein said compound is present in an amount to measurably degrade and/or inhibit STAT6 or a mutant thereof.

Compounds according to the invention are useful in the treatment of inflammatory or obstructive airways diseases, resulting, for example, in reduction of tissue damage, airways inflammation, bronchial hyperreactivity, remodeling or disease progression. Inflammatory or obstructive airways diseases to which the present invention is applicable include asthma of whatever type or genesis including both intrinsic (non-allergic) asthma and extrinsic (allergic) asthma, mild asthma, moderate asthma, severe asthma, bronchitic asthma, exercise-induced asthma, occupational asthma and asthma exacerbated or induced following bacterial or viral infection. Treatment of asthma is also to be understood as embracing treatment of subjects, e.g., of less than 4 or 5 years of age, exhibiting wheezing symptoms and diagnosed or diagnosable as “wheezy infants”, an established patient category of major medical concern and now often identified as incipient or early-phase asthmatics.

Another aspect of the present invention relates to a method of treating an allergic or inflammatory disease in a subject comprising administering to the subject a therapeutically effective amount of a compound of the present invention to the subject. The disease may be a lung disease such as, e.g., asthma, airway hyperresponsiveness (AHR), an allergic disease, allergic rhinitis, emphysema, chronic obstructive pulmonary disease (COPD), reactive airway disease, chronic rhinosinusitis, or essentially any other disease of the upper or lower airways that produces airflow obstruction.

Prophylactic efficacy in the treatment of asthma will be evidenced by reduced frequency or severity of symptomatic attack, e.g., of acute asthmatic or bronchoconstrictor attack, improvement in lung function or improved airways hyperreactivity. It may further be evidenced by reduced requirement for other, symptomatic therapy, such as therapy for or intended to restrict or abort symptomatic attack when it occurs, for example antiinflammatory or bronchodilatory. Prophylactic benefit in asthma may in particular be apparent in subjects prone to “morning dipping”. “Morning dipping” is a recognized asthmatic syndrome, common to a substantial percentage of asthmatics and characterized by asthma attack, e.g., between the hours of about 4 to 6 am, i.e., at a time normally substantially distant form any previously administered symptomatic asthma therapy.

In some embodiments, STAT6, via its Src homology 2 (SH2) domain, is recruited to the phosphotyrosine residues and is phosphorylated on Tyr641. In some embodiments, STAT6 then dimerizes via reciprocal SH2 domain-pTyr641 interactions, translocates to the nucleus, and participates in the expression of genes leading to asthma and airway hyperresponsiveness (AHR).

In some embodiments, the present invention provides a method of treating asthma in a patient in need thereof, comprising administering a compound of the present invention, or a pharmaceutically acceptable salt thereof.

In some embodiments, the present invention provides a method of treating airway hyperresponsiveness (AHR) in a patient in need thereof, comprising administering a compound of the present invention, or a pharmaceutically acceptable salt thereof.

In some embodiments, the present invention provides a method of treating allergic rhinitis in a patient in need thereof, comprising administering a compound of the present invention, or a pharmaceutically acceptable salt thereof.

In some embodiments, the present invention provides a method of treating allergic asthma in a patient in need thereof, comprising administering a compound of the present invention, or a pharmaceutically acceptable salt thereof.

In some embodiments, the present invention provides a method of treating emphysema in a patient in need thereof, comprising administering a compound of the present invention, or a pharmaceutically acceptable salt thereof.

In some embodiments, the present invention provides a method of treating chronic rhinosinusitis (e.g., with nasal polyposis) in a patient in need thereof, comprising administering a compound of the present invention, or a pharmaceutically acceptable salt thereof.

In some embodiments, the present invention provides a method oftreating atopic dermatitis in a patient in need thereof, comprising administering a compound of the present invention, or a pharmaceutically acceptable salt thereof.

In some embodiments, the present invention provides a method of treating COPD in a patient in need thereof, comprising administering a compound of the present invention, or a pharmaceutically acceptable salt thereof.

Compounds of the current invention can be used for other inflammatory or obstructive airways diseases and conditions to which the present invention is applicable and include acute lung injury (ALI), adult/acute respiratory distress syndrome (ARDS), chronic obstructive pulmonary, airways or lung disease (COPD, COAD or COLD), including chronic bronchitis or dyspnea associated therewith, emphysema, as well as exacerbation of airways hyperreactivity consequent to other drug therapy, in particular other inhaled drug therapy. The invention is also applicable to the treatment of bronchitis of whatever type or genesis including, but not limited to, acute, arachidic, catarrhal, croupus, chronic or phthinoid bronchitis. Further inflammatory or obstructive airways diseases to which the present invention is applicable include pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis.

With regard to their anti-inflammatory activity, in particular in relation to inhibition of eosinophil activation, compounds of the invention are also useful in the treatment of eosinophil related disorders, e.g., eosinophilia, in particular eosinophil related disorders of the airways (e.g., involving morbid eosinophilic infiltration of pulmonary tissues) including hypereosinophilia as it effects the airways and/or lungs as well as, for example, eosinophil-related disorders of the airways consequential or concomitant to Loffler's syndrome, eosinophilic pneumonia, parasitic (in particular metazoan) infestation (including tropical eosinophilia), bronchopulmonary aspergillosis, esophagitis, polyarteritis nodosa (including Churg-Strauss syndrome), eosinophilic granuloma and eosinophil-related disorders affecting the airways occasioned by drug-reaction.

In some embodiments, the present invention provides a method of treating eosinophilic esophagitis in a patient in need thereof, comprising administering a compound of the present invention, or a pharmaceutically acceptable salt thereof.

In some embodiments, the present invention provides a method of treating prurigo nodularis in a patient in need thereof, comprising administering a compound of the present invention, or a pharmaceutically acceptable salt thereof.

Compounds of the invention are also useful in the treatment of inflammatory or allergic conditions of the skin. In some embodiments, the present invention provides a method of treating inflammatory or allergic conditions of the skin in a patient in need thereof, comprising administering a compound of the present invention, or a pharmaceutically acceptable salt thereof.

In some embodiments the inflammatory disease of the skin is selected from psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, systemic lupus erythematosus, pemphigus vulgaris, pemphigus foliaceus, paraneoplastic pemphigus, epidermolysis bullosa acquisita, acne vulgaris, and other inflammatory or allergic conditions of the skin.

Compounds of the invention may also be used for the treatment of other diseases or conditions, such as diseases or conditions having an inflammatory component, for example, treatment of diseases and conditions of the eye such as ocular allergy, conjunctivitis, keratoconjunctivitis sicca, and vernal conjunctivitis, diseases affecting the nose including allergic rhinitis, and inflammatory disease in which autoimmune reactions are implicated or having an autoimmune component or etiology, including autoimmune hematological disorders (e.g. hemolytic anemia, aplastic anemia, pure red cell anemia and idiopathic thrombocytopenia), systemic lupus erythematosus, rheumatoid arthritis, polychondritis, scleroderma, Wegener granulamatosis, dermatomyositis, chronic active hepatitis, myasthenia gravis, Steven-Johnson syndrome, idiopathic sprue, autoimmune inflammatory bowel disease (e.g., ulcerative colitis and Crohn's disease), irritable bowel syndrome, celiac disease, periodontitis, hyaline membrane disease, kidney disease, glomerular disease, alcoholic liver disease, multiple sclerosis, endocrine opthalmopathy, Grave's disease, sarcoidosis, alveolitis, chronic hypersensitivity pneumonitis, multiple sclerosis, primary biliary cirrhosis or primary biliary cholangitis, uveitis (anterior and posterior), Sjogren's syndrome, keratoconjunctivitis sicca and vernal keratoconjunctivitis, interstitial lung disease or fibrosis, psoriatic arthritis, systemic juvenile idiopathic arthritis, cryopyrin-associated periodic syndrome, nephritis, vasculitis, diverticulitis, interstitial cystitis, glomerulonephritis (with and without nephrotic syndrome, e.g. including idiopathic nephrotic syndrome or minal change nephropathy), chronic granulomatous disease, endometriosis, leptospiriosis renal disease, glaucoma, retinal disease, ageing, headache, pain, complex regional pain syndrome, cardiac hypertrophy, musclewasting, catabolic disorders, obesity, fetal growth retardation, hyperchlolesterolemia, heart disease, chronic heart failure, mesothelioma, anhidrotic ecodermal dysplasia, Behcet's disease, incontinentia pigmenti, Paget's disease, pancreatitis, hereditary periodic fever syndrome, asthma (allergic and non-allergic, mild, moderate, severe, bronchitic, and exercise-induced), acute lung injury, acute respiratory distress syndrome, eosinophilia, hypersensitivities, anaphylaxis, nasal sinusitis, ocular allergy, silica induced diseases, COPD (reduction of damage, airways inflammation, bronchial hyperreactivity, remodeling or disease progression), pulmonary disease, cystic fibrosis, acid-induced lung injury, pulmonary hypertension, polyneuropathy, cataracts, muscle inflammation in conjunction with systemic sclerosis, inclusion body myositis, myasthenia gravis, thyroiditis. Addison's disease, lichen planus, Type 1 diabetes, or Type 2 diabetes, appendicitis, atopic dermatitis, asthma, allergy, blepharitis, bronchiolitis, bronchitis, bursitis, cervicitis, cholangitis, cholecystitis, chronic graft rejection, colitis, conjunctivitis, Crohn's disease, cystitis, dacryoadenitis, dernatitis, dennatomyositis, prurigo nodularis, encephalitis, encephalomyelitis, endocarditis, endometritis, enteritis, enterocolitis, epicondylitis, epididymitis, fasciitis, fibrositis, gastritis, gastroenteritis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pycloncphritis, rhinitis, salpingitis, sinusitis, stomatitis, synovitis, tendonitis, tonsillitis, ulcerative colitis, uveitis, vaginitis, vasculitis, or vulvitis.

In some embodiments, the present invention provides a method of treating an autoimmune disease selected from encephalomyclitis, systemic sclerosis, idiopathic pulmonary fibrosis (IPF), inflammatory bowel disease, atopic dennatitis, rheumatoid arthritis, graft versus host disease (acute and chronic), and other tissue fibrosis diseases.

In some embodiments, the present invention provides a method of treating idiopathic interstitial pneumonia(s) (IIPs), including any type of lung fibrosis, either interstitial lung disease associated with rheumatic disease (including SSc) or IPF itself, in a patient in need thereof, comprising administering a compound of the present invention, or a pharmaceutically acceptable salt thereof.

In some embodiments the inflammatory disease which can be treated according to the methods of this invention is selected from acute and chronic gout, chronic gouty arthritis, psoriasis, psoriatic arthritis, rheumatoid arthritis, Juvenile rheumatoid arthritis, systemic juvenile idiopathic arthritis (SJIA), cryopyrin associated periodic syndrome (CAPS), and osteoarthritis.

In some embodiments the inflammatory disease which can be treated according to the methods of this invention is a TH17 mediated disease or TH17-associated disease. In some embodiments the TH17 mediated disease or TH17-associated disease is selected from psoriasis, psoriatric arthritis, systemic lupus erythematosus, multiple sclerosis, and inflammatory bowel disease (including Crohn's disease or ulcerative colitis), or graft-versus-host disease.

In some embodiments the inflammatory disease which can be treated according to the methods of this invention is selected from Sjogren's syndrome, allergic disorders, osteoarthritis, conditions of the eye such as ocular allergy, conjunctivitis, keratoconjunctivitis sicca and vernal conjunctivitis, and diseases affecting the nose such as allergic rhinitis.

In some embodiments, the present invention provides a method of treating an autoimmune disease or inflammatory disorder is selected from nonalcoholic fatty liver disease (NAFLD), nonalcoholic steatohepatitis (NASH), idiopathic autoimmune hepatitis, progressive fibrosis associated interstitial lung disease, pulmonary arterial hypertension (PAH), immunoglobulin G4-related disease (IgG4-RD), chronic organ rejection (e.g., lung transplant), vasculitides (e.g., vasculitides), and STAT6 gain of function (GOF) mutations.

In some embodiments, the present invention provides a method of treating STAT6 gain of function (GOF) mutations in a patient in need thereof, comprising administering a compound of the present invention, or a pharmaceutically acceptable salt thereof. In some embodiments, the STAT6 GOF mutation is STAT6VT.

In some embodiments, the cardiovascular disease which can be treated according to the methods of the present invention include, but are not limited to, restenosis, cardiomegaly, atherosclerosis, myocardial infarction, ischemic stroke, congestive heart failure, angina pectoris, reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, and deep venous thrombosis.

In some embodiments, the neurodegenerative disease which can be treated according to the methods of the present invention include, but are not limited to, Alzheimer's disease, Parkinson's disease, amyotrophic lateral sclerosis, Huntington's disease, cerebral ischemia, and neurodegenerative disease caused by traumatic injury, glutamate neurotoxicity, hypoxia, epilepsy, treatment of diabetes, metabolic syndrome, obesity, organ transplantation and graft versus host disease.

In some embodiments the invention provides a method of treating, preventing or lessening the severity of Alzheimer's disease comprising administering to a patient in need thereof a provided compound or a pharmaceutically acceptable salt or composition thereof.

In some embodiments the invention provides a method of treating a disease or condition commonly occurring in connection with transplantation. In some embodiments, the disease or condition commonly occurring in connection with transplantation is selected from organ transplantation, organ transplant rejection, and graft versus host disease.

In some embodiments the invention provides a method of treating a metabolic disease. In some embodiments the metabolic disease is selected from Type 1 diabetes, Type 2 diabetes, metabolic syndrome, and obesity.

In some embodiments the invention provides a method of treating a viral disease. In some embodiments, the viral infection is HIV or COVID19 infection.

In some embodiments, the aberrant activation of STAT6 which can be treated according to the methods of this invention is a human cancer. In some embodiments, the human cancer which can be treated according to the methods of this invention include benign or malignant tumor, solid tumor, liquid tumor, carcinoma of the brain, kidney, liver, adrenal gland, bladder, breast, stomach, gastric tumors, ovaries, colon, rectum, prostate, pancreas, lung, vagina, cervix, testis, genitourinary tract, esophagus, larynx, skin, bone or thyroid, sarcoma, glioblastomas, neuroblastomas, multiple myeloma, gastrointestinal cancer, especially colon carcinoma or colorectal adenoma, a tumor of the neck and head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, non-small-cell lung carcinoma, lymphomas, Hodgkin's and Non-Hodgkin's, a mammary carcinoma, follicular carcinoma, undifferentiated carcinoma, papillary carcinoma, seminoma, melanoma, an IL-1 driven disorder, an MyD88 driven disorder, Smoldering of indolent multiple myeloma, or hematological malignancies (including leukemia, diffuse large B-cell lymphoma (DLBCL), ABC DLBCL, chronic lymphocytic leukemia (CLL), chronic lymphocytic lymphoma, primary effusion lymphoma, Burkitt lymphoma/leukemia, acute lymphocytic leukemia, B-cell prolymphocytic leukemia, lymphoplasmacytic lymphoma, Waldenstrom's macroglobulinemia (WM), splenic marginal zone lymphoma, multiple myeloma, plasmacytoma, intravascular large B-cell lymphoma).

In some embodiments, the present invention provides a method of treating a cancer selected from glioma, breast cancer, prostate cancer, head and neck squamous cell carcinoma, skin melanomas, ovarian cancer, malignant peripheral nerve shealth tumors (MPNST), pancreatic cancer, non-small cell lung cancer (NSCLC) including EGFR-mutant NSCLC, urothelial cancer, liver cancer, bile duct cancer, kidney cancer, colon cancer, esophageal cancer, gastric cancer, gastrointestinal stromal tumors, and hematological malignancies include lymphomas, leukemias, myelomas, myeloproliferative neoplasms and myelodysplastic syndromes.

In some embodiments, the present invention provides a method of treating a JAK-associated disease. In some embodiments, the JAK-associated disease is cancer including those characterized by solid tumors (e.g., prostate cancer, renal cancer, hepatic cancer, pancreatic cancer, gastric cancer, breast cancer, lung cancer, cancers of the head and neck, thyroid cancer, glioblastoma, Kaposi's sarcoma, Castleman's disease, uterine leiomyosarcoma, melanoma etc.), hematological cancers (e.g., lymphoma, leukemia Such as acute lymphoblastic leukemia (ALL), acute myelogenous leukemia (AML) or multiple myeloma), and skin cancer such as cutaneous T-cell lymphoma (CTCL) and cutaneous B-cell lymphoma. Example CTCLs include Sezary syndrome and mycosis fungoides.

In some embodiments, the present invention provides a method of treating a hematologic malignancy selected from LGL leukemia (T and NK cell), cutaneous T cell lymphoma (CTCL), peripheral T cell lymphomas (PTCL, all subtypes including ALCL), diffuse large B cell lymphoma (DLBCL), acute myelogenous leukemia, multiple myeloma, and myelofibrosis.

Furthermore, the invention provides the use of a compound according to the definitions herein, or a pharmaceutically acceptable salt, or a hydrate or solvate thereof for the preparation of a medicament for the treatment of a proliferative disease, an inflammatory disease, an obstructive respiratory disease, a cardiovascular disease, a metabolic disease, a neurological disease, a neurodegenerative disease, a viral disease, or a disorder commonly occurring in connection with transplantation.

### Combination Therapies

Depending upon the particular condition, or disease, to be treated, additional therapeutic agents, which are normally administered to treat that condition, may be administered in combination with compounds and compositions of this invention. As used herein, additional therapeutic agents that are normally administered to treat a particular disease, or condition, are known as “appropriate for the disease, or condition, being treated.”

In certain embodiments, a provided combination, or composition thereof, is administered in combination with another therapeutic agent.

In some embodiments, the present invention provides a method of treating a disclosed disease or condition comprising administering to a patient in need thereof an effective amount of a compound disclosed herein or a pharmaceutically acceptable salt thereof and co-administering simultaneously or sequentially an effective amount of one or more additional therapeutic agents, such as those described herein. In some embodiments, the method includes co-administering one additional therapeutic agent. In some embodiments, the method includes co-administering two additional therapeutic agents. In some embodiments, the combination of the disclosed compound and the additional therapeutic agent or agents acts synergistically.

Examples of agents the combinations of this invention may also be combined with include, without limitation: treatments for Alzheimer's Disease such as Aricept® and Excelon®; treatments for HIV such as ritonavir; treatments for Parkinson's Disease such as L-DOPA/carbidopa, entacapone, ropinrole, pramipexole, bromocriptine, pergolide, trihexephendyl, and amantadine; agents for treating Multiple Sclerosis (MS) such as beta interferon (e.g., Avonex® and Rebif®), glatiramer acetate (Copaxone®), and mitoxantrone; treatments for asthma such as albuterol and Singulair©; agents for treating schizophrenia such as zyprexa, risperdal, seroquel, and haloperidol; anti-inflammatory agents such as corticosteroids, TNF blockers, IL-1 R^{A}, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophophamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; agents that prolong or improve pharmacokinetics such as cytochrome P450 inhibitors (i.e., inhibitors of metabolic breakdown) and CYP3A4 inhibitors (e.g., ketokenozole and ritonavir), pirfenidone (Esbriet®), nintedanib (Ofev©), intravenous immunoglobulins, bosentan (Tracleer®), nifedipine (Procardia XL®), sildenafil (Revatio®), losartan (Cozaar®), iloprost (Ventavis®), topical nitroglycerin, N-acetylcvsteine, antiacid therapy, and agents for treating immunodeficiency disorders such as gamma globulin.

In certain embodiments, combination therapies of the present invention, or a pharmaceutically acceptable composition thereof, are administered in combination with a monoclonal antibody or an siRNA therapeutic.

Those additional agents may be administered separately from a provided combination therapy, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this invention. For example, a combination of the present invention may be administered with another therapeutic agent simultaneously or sequentially in separate unit dosage forms or together in a single unit dosage form.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

One or more other therapeutic agent may be administered separately from a compound or composition of the invention, as part of a multiple dosage regimen. Alternatively, one or more other therapeutic agents may be part of a single dosage form, mixed together with a compound of this invention in a single composition. If administered as a multiple dosage regime, one or more other therapeutic agent and a compound or composition of the invention may be administered simultaneously, sequentially or within a period of time from one another, for example within 1, 2, 3, 4, 5, 6, 7, 8, 9, 10, 11, 12, 13, 14, 15, 16, 17, 18, 18, 20, 21, 22, 23, or 24 hours from one another. In some embodiments, one or more other therapeutic agent and a compound or composition of the invention are administered as a multiple dosage regimen within greater than 24 hours apart.

In one embodiment, the present invention provides a composition comprising a provided compound and one or more additional therapeutic agents. The therapeutic agent may be administered together with a provided compound, or may be administered prior to or following administration of a provided compound. Suitable therapeutic agents are described in further detail below. In certain embodiments, a provided compound may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours before the therapeutic agent. In other embodiments, a provided compound may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours following the therapeutic agent.

In another embodiment, the present invention provides a method of treating an inflammatory disease, disorder or condition by administering to a patient in need thereof a provided compound and one or more additional therapeutic agents. Such additional therapeutic agents may be small molecules or recombinant biologic agents and include, for example, acetaminophen, non-steroidal anti-inflammatory drugs (NSAIDS) such as aspirin, ibuprofen, naproxen, etodolac (Lodine®) and celecoxib, colchicine (Colcrys®), corticosteroids such as prednisone, prednisolone, methylprednisolone, hydrocortisone, and the like, probenecid, allopurinol, febuxostat (Uloric®), sulfasalazine (Azulfidine®), antimalarials such as hydroxychloroquine (Plaquenil®) and chloroquine (Aralen®), methotrexate (Rheumatrex®)), gold salts such as gold thioglucose (SolganalD3), gold thiomalate (Myochrysine®)) and auranofin (Ridaura®)), D-penicillamine (Depen® or Cuprimine®), azathioprine (Imuran®), cyclophosphamide (Cytoxan®), chloranbucil (Leukeran®), cyclosporine (Sandimmune®), leflunomide (Arava®) and “anti-TNF” agents such as etanercept (Enbrel®), infliximab (Remicade®), golimumab (Simponi®), certolizumab pegol (Cimzia®) and adalimumab (Humira®), “anti-IL-1” agents such as anakinra (Kineret®) and rilonacept (Arcalyst®), canakinumab (Ilaris®), anti-Jak inhibitors such as tofacitinib, antibodies such as rituximab (Rituxan®), “anti-T-cell” agents such as abatacept (Orencia®), “anti-IL-6” agents such as tocilizumab (Actemra(®), diclofenac, cortisone, hyaluronic acid (Synvisc® or Hyalgan®), monoclonal antibodies such as tanezumab, anticoagulants such as heparin (Calcinparine® or Liquaemin®)) and warfarin (Coumadin®), antidiarrheals such as diphenoxylate (Lomotil®) and loperamide (Imodium®), bile acid binding agents such as cholestyramine, alosetron (Lotronex®), lubiprostone (Amitiza®), laxatives such as Milk of Magnesia, polyethylene glycol (MiraLax®), Dulcolax(D, Correctol® and Senokot®, anticholinergics or antispasmodics such as dicyclomine (Bentyl), Singulair®, beta-2 agonists such as albuterol (Ventolin® HFA, Proventil®, HFA), levalbuterol (Xopenex®3), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), inhaled corticosteroids such as beclomethasone dipropionate (Beclovent®, Qvar®, and Vanceril®), triamcinolone acetonide (Azmacort®), mometasone (Asthmanex®), budesonide (Pulmocort®), and flunisolide (Aerobid®), Afviar®, Symbicort®, Dulera®, cromolyn sodium (Intal®), methylxanthines such as theophylline (Theo-Dur®, Theolair®. Slo-bid®, Uniphyl®, Theo-24®) and aminophylline, IgE antibodies such as omalizumab (Xolair®), nucleoside reverse transcriptase inhibitors such as zidovudine (Retrovir®), abacavir (Ziagen®), abacavir/lamivudine (Epzicom®), abacavir/lamivudine/zidovudine (Trizivir®), didanosine (Videx®), emtricitabine (Emtriva®), lamivudine (Epivir®), lamivudine/zidovudine (Combivir®), stavudine (Zerit®), and zalcitabine (Hivid®), non-nucleoside reverse transcriptase inhibitors such as delavirdine (Rescriptor®), efavirenz (Sustiva®), nevairapine (Viramune®) and etravirine (Intelence®), nucleotide reverse transcriptase inhibitors such as tenofovir (Viread®), protease inhibitors such as amprenavir (Agenerase®), atazanavir (Reyataz®), darunavir (Prezista®), fosamprenavir (Lexiva®), indinavir (Crixivan®), lopinavir and ritonavir (Kaletra®), nelfinavir (Viracept®), ritonavir (Norvir®), saquinavir (Fortovase® or Invirase®), and tipranavir (Aptivus®), entry inhibitors such as enfuvirtide (Fuzeon®) and maraviroc (Selzentry®), integrase inhibitors such as raltegravir (Isentress®), doxorubicin (Hydrodaunorubicin®), vincristine (Oncovin®), bortezomib (Velcade®), and dexamethasone (Decadron®) in combination with lenalidomide (Revlimid®), or any combination(s) thereof.

In another embodiment, the present invention provides a method of treating gout comprising administering to a patient in need thereof a provided compound and one or more additional therapeutic agents selected from non-steroidal anti-inflammatory drugs (NSAIDS) such as aspirin, ibuprofen, naproxen, etodolac (Lodine®) and celecoxib, colchicine (Colcrys®), corticosteroids such as prednisone, prednisolone, methylprednisolone, hydrocortisone, and the like, probenecid, allopurinol and febuxostat (Uloric®).

In another embodiment, the present invention provides a method of treating rheumatoid arthritis comprising administering to a patient in need thereof a provided compound and one or more additional therapeutic agents selected from non-steroidal anti-inflammatory drugs (NSAIDS) such as aspirin, ibuprofen, naproxen, etodolac (Lodine®) and celecoxib, corticosteroids such as prednisone, prednisolone, methylprednisolone, hydrocortisone, and the like, sulfasalazine (Azulfidine®), antimalarials such as hydroxychloroquine (Plaquenil®) and chloroquine (Aralen®), methotrexate (Rheumatrex®), gold salts such as gold thioglucose (Solganal®), gold thiomalate (Myochrysine®) and auranofin (Ridaura®), D-penicillamine (Depen® or Cuprimine®), azathioprine (Imuran®), cyclophosphamide (Cytoxan®), chlorambucil (Leukeran®), cyclosporine (Sandimmune®), leflunomide (Arava®) and “anti-TNF” agents such as etanercept (Enbrel®), infliximab (Remicade®), golimumab (Simponi®), certolizumab pegol (Cimzia®) and adalimumab (Humira®), “anti-IL-1” agents such as anakinra (Kineret®) and rilonacept (Arcalyst®), antibodies such as rituximab (Rituxan®), “anti-T-cell” agents such as abatacept (Orencia®) and “anti-IL-6” agents such as tocilizumab (Actemra®).

In some embodiments, the present invention provides a method of treating osteoarthritis comprising administering to a patient in need thereof a provided compound and one or more additional therapeutic agents selected from acetaminophen, non-steroidal anti-inflammatory drugs (NSAIDS) such as aspirin, ibuprofen, naproxen, etodolac (Lodine®) and celecoxib, diclofenac, cortisone, hyaluronic acid (Synvisc®, or Hvalgan®) and monoclonal antibodies such as tanezumab.

In some embodiments, the present invention provides a method of treating lupus comprising administering to a patient in need thereof a provided compound and one or more additional therapeutic agents selected from acetaminophen, non-steroidal anti-inflammatory drugs (NSAIDS) such as aspirin, ibuprofen, naproxen, etodolac (Lodine®) and celecoxib, corticosteroids such as prednisone, prednisolone, methylprednisolone, hydrocortisone, and the like, antimalarials such as hvdroxychloroquine (Plaquenil®) and chloroquine (Aralen®), cyclophosphamide (Cytoxan®)), methotrexate (Rheumatrex®), azathioprine (Imuran®) and anticoagulants such as heparin (Calcinparine® or Liquaemin®) and warfarin (Coumadin®).

In some embodiments, the present invention provides a method of treating inflammatory bowel disease comprising administering to a patient in need thereof a provided compound and one or more additional therapeutic agents selected from mesalamine (Asacol®) sulfasalazine (Azulfidine®), antidiarrheals such as diphenoxylate (Lomotil®) and loperamide (Imodium®), bile acid binding agents such as cholestyramine, alosetron (Lotronex®), lubiprostone (Amitiza®), laxatives such as Milk of Magnesia, polyethylene glycol (MiraLax®), Dulcolax®, Correctol® and Senokot® and anticholinergics or antispasmodics such as dicyclomine (Bentyl®), anti-TNF therapies, steroids, and antibiotics such as Flagyl or ciprofloxacin.

In some embodiments, the present invention provides a method of treating asthma comprising administering to a patient in need thereof a provided compound and one or more additional therapeutic agents selected from Singulair®, beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), inhaled corticosteroids such as prednisone, prednisolone, beclomethasone dipropionate (Beclovent®, Qvar®, and Vanceril®), triamcinolone acetonide (Azmacort®), mometasone (Asthmanex®), budesonide (Pulmocort®), flunisolide (Aerobid®), Afviar®, Symbicort®, and Dulera®, cromolyn sodium (Intal®), methylxanthines such as theophylline (Theo-Dur®, Theolair®, Slo-bid®, Uniphyl®, Theo-24®) and aminophylline, and IgE antibodies such as omalizumab (Xolair®).

In some embodiments, the present invention provides a method of treating COPD comprising administering to a patient in need thereof a provided compound and one or more additional therapeutic agents selected from beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), methylxanthines such as theophylline (Theo-Dur®, Theolair®, Slo-bid®, Uniphyl®, Theo-24®) and aminophylline, inhaled corticosteroids such as prednisone, prednisolone, beclomethasone dipropionate (Beclovent®, Qvar®, and Vanceril®), triamcinolone acetonide (Azmacort®), mometasone (Asthmanex®), budesonide (Pulmocort®), flunisolide (Aerobid®), Afviar®, Symbicort®, and Dulera®,

In some embodiments, the present invention provides a method of treating HIV comprising administering to a patient in need thereof a provided compound and one or more additional therapeutic agents selected from nucleoside reverse transcriptase inhibitors such as zidovudine (Retrovir®), abacavir (Ziagen®), abacavir/lamivudine (Epzicom®), abacavir/lamivudine/zidovudine (Trizivir®), didanosine (Videx®), emtricitabine (Emtriva®), lamivudine (Epivir®), lamivudine/zidovudine (Combivir®), stavudine (Zerit®), and zalcitabine (Hivid®), non-nucleoside reverse transcriptase inhibitors such as delavirdine (Rescriptor®), efavirenz (Sustiva®), nevairapine (Viramune®) and etravirine (Intelence®), nucleotide reverse transcriptase inhibitors such as tenofovir (Viread®), protease inhibitors such as amprenavir (Agenerase®), atazanavir (Reyataz®), darunavir (Prezista®), fosamprenavir (Lexiva®), indinavir (Crixivan®), lopinavir and ritonavir (Kaletra®)), nelfinavir (Viracept®), ritonavir (Norvir®), saquinavir (Fortovase® or Invirase®), and tipranavir (Aptivus®), entry inhibitors such as enfuvirtide (Fuzeon®) and maraviroc (Selzentry®), integrase inhibitors such as raltegravir (Isentress®), and combinations thereof.

In another embodiment, the present invention provides a method of treating a hematological malignancy comprising administering to a patient in need thereof a provided compound and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, a BTK inhibitor, a JAK/pan-JAK inhibitor, a TYK2 inhibitor, a PI3K inhibitor, a SYK inhibitor, and combinations thereof.

In another embodiment, the present invention provides a method of treating a solid tumor comprising administering to a patient in need thereof a provided compound and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, a BTK inhibitor, a JAK/pan-JAK inhibitor, a TYK2 inhibitor, a PI3K inhibitor, a SYK inhibitor, and combinations thereof.

In another embodiment, the present invention provides a method of treating a hematological malignancy comprising administering to a patient in need thereof a provided compound and a Hedgehog (Hh) signaling pathway inhibitor. In some embodiments, the hematological malignancy is DLBCL (Ramirez et al “Defining causative factors contributing in the activation of hedgehog signaling in diffuse large B-cell lymphoma” Leuk. Res. (2012), published online July 17, and incorporated herein by reference in its entirety).

In another embodiment, the present invention provides a method of treating diffuse large B-cell lymphoma (DLBCL) comprising administering to a patient in need thereof a provided compound and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, and combinations thereof.

In another embodiment, the present invention provides a method of treating multiple myeloma comprising administering to a patient in need thereof a provided compound and one or more additional therapeutic agents selected from bortezomib (Velcade®), and dexamethasone (Decadron®), a hedgehog signaling inhibitor, a BTK inhibitor, a JAK/pan-JAK inhibitor, a TYK2 inhibitor, a PI3K inhibitor, a SYK inhibitor in combination with lenalidomide (Revlimid®).

In another embodiment, the present invention provides a method of treating Waldenstrom's macroglobulinemia comprising administering to a patient in need thereof a provided compound and one or more additional therapeutic agents selected from chlorambucil (Leukeran®), cyclophosphamide (Cytoxan®, Neosar®), fludarabine (Fludara®), cladribine (Leustatin®), rituximab (Rituxan®), a hedgehog signaling inhibitor, a BTK inhibitor, a JAK/pan-JAK inhibitor, a TYK2 inhibitor, a PI3K inhibitor, and a SYK inhibitor.

In some embodiments, one or more other therapeutic agent is an antagonist of the hedgehog pathway. Approved hedgehog pathway inhibitors which may be used in the present invention include sonidegib (Odomzo®, Sun Pharmaceuticals); and vismodegib (Erivedge®, Genentech), both for treatment of basal cell carcinoma.

In some embodiments, one or more other therapeutic agent is a Poly ADP ribose polymerase (PARP) inhibitor. In some embodiments, a PARP inhibitor is selected from olaparib (Lynparza®, AstraZeneca): rucaparib (Rubraca®, Clovis Oncology); niraparib (Zejula®, Tesaro); talazoparib (MDV3800/BMN 673/LT00673, Medivation/Pfizer/Biomarin); veliparib (ABT-888, AbbVie); and BGB-290 (BeiGene, Inc.).

In some embodiments, one or more other therapeutic agent is a histone deacetylase (HDAC) inhibitor. In some embodiments, an HDAC inhibitor is selected from vorinostat (Zolinza®, Merck); romidepsin (Istodax®, Celgene); panobinostat (Farydak®, Novartis); belinostat (Beleodaq®, Spectrum Pharmaceuticals); entinostat (SNDX-275, Syndax Pharmaceuticals) (NCT00866333); and chidamide (Epidaza®, HBI-8000, Chipscreen Biosciences, China).

In some embodiments, one or more other therapeutic agent is a CDK inhibitor, such as a CDK4/CDK6 inhibitor. In some embodiments, a CDK 4/6 inhibitor is selected from palbociclib (Ibrance®, Pfizer); ribociclib (Kisqali®, Novartis): abemaciclib (Ly2835219, Eli Lilly); and trilaciclib (G1T28, G1 Therapeutics).

In some embodiments, one or more other therapeutic agent is a folic acid inhibitor. Approved folic acid inhibitors useful in the present invention include pemetrexed (Alimta®, Eli Lilly).

In some embodiments, one or more other therapeutic agent is a CC chemokine receptor 4 (CCR4) inhibitor. CCR4 inhibitors being studied that may be useful in the present invention include mogamulizumab (Poteligeo®, Kyowa Hakko Kirin, Japan).

In some embodiments, one or more other therapeutic agent is an isocitrate dehydrogenase (IDH) inhibitor. IDH inhibitors being studied which may be used in the present invention include AG120 (Celgene; NCT02677922); AG221 (Celgene, NCT02677922; NCT02577406); BAY1436032 (Bayer, NCT02746081): IDH305 (Novartis, NCT02987010).

In some embodiments, one or more other therapeutic agent is an arginase inhibitor. Arginase inhibitors being studied which may be used in the present invention include AEB1102 (pegylated recombinant arginase, Aeglea Biotherapeutics), which is being studied in Phase 1 clinical trials for acute myeloid leukemia and myelodysplastic syndrome (NCT02732184) and solid tumors (NCT02561234); and CB-1158 (Calithera Biosciences).

In some embodiments, one or more other therapeutic agent is a glutaminase inhibitor. Glutaminase inhibitors being studied which may be used in the present invention include CB-839 (Calithera Biosciences).

In some embodiments, one or more other therapeutic agent is an antibody that binds to tumor antigens, that is, proteins expressed on the cell surface of tumor cells. Approved antibodies that bind to tumor antigens which may be used in the present invention include rituximab (Rituxan®, Genentech/BiogenIdec); ofatumnumab (anti-CD20, Arzerra®, GlaxoSmithKline); obinutuzumab (anti-CD20, Gazyva®, Genentech), ibritumomab (anti-CD20 and Yttrium-90, Zevalin®, Spectrum Pharmaceuticals); daratumumab (anti-CD38, Darzalex®, Janssen Biotech), dinutuximab (anti-glycolipid GD2, Unituxin®, United Therapeutics); trastuzumab (anti-HER2, Herceptin®, Genentech): ado-trastuzumab emtansine (anti-HER2, fused to emtansine, Kadcyla®, Genentech); and pertuzumab (anti-HER2, Perjeta®, Genentech); and brentuximab vedotin (anti-CD30-drug conjugate, Adcetris®, Seattle Genetics).

In some embodiments, one or more other therapeutic agent is a topoisomerase inhibitor. Approved topoisomerase inhibitors useful in the present invention include irinotecan (Onivyde®, Merrimack Pharmaceuticals); topotecan (Hycamtin®, GlaxoSmithKline). Topoisomerase inhibitors being studied which may be used in the present invention include pixantrone (Pixuvri®, CTI Biopharma).

In some embodiments, one or more other therapeutic agent is an inhibitor of anti-apoptotic proteins, such as BCL-2. Approved anti-apoptotics which may be used in the present invention include venetoclax (Venclexta®, AbbVie/Genentech); and blinatumomab (Blincyto®, Amgen). Other therapeutic agents targeting apoptotic proteins which have undergone clinical testing and may be used in the present invention include navitoclax (ABT-263, Abbott), a BCL-2 inhibitor (NCT02079740).

In some embodiments, one or more other therapeutic agent is an androgen receptor inhibitor. Approved androgen receptor inhibitors useful in the present invention include enzalutamide (Xtandi®, Astellas/Medivation); approved inhibitors of androgen synthesis include abiraterone (Zytiga®, Centocor/Ortho); approved antagonist of gonadotropin-releasing hormone (GnRH) receptor (degaralix, Firmagon®, Ferring Pharmaceuticals).

In some embodiments, one or more other therapeutic agent is a selective estrogen receptor modulator (SERM), which interferes with the synthesis or activity of estrogens. Approved SERMs useful in the present invention include raloxifene (Evista®, Eli Lilly).

In some embodiments, one or more other therapeutic agent is an inhibitor of bone resorption. An approved therapeutic which inhibits bone resorption is Denosumab (Xgeva®, Amgen), an antibody that binds to RANKL, prevents binding to its receptor RANK, found on the surface of osteoclasts, their precursors, and osteoclast-like giant cells, which mediates bone pathology in solid tumors with osseous metastases. Other approved therapeutics that inhibit bone resorption include bisphosphonates, such as zoledronic acid (Zometa®, Novartis).

In some embodiments, one or more other therapeutic agent is an inhibitor of interaction between the two primary p53 suppressor proteins, MDMX and MDM2. Inhibitors of p53 suppression proteins being studied which may be used in the present invention include ALRN-6924 (Aileron), a stapled peptide that equipotently binds to and disrupts the interaction of MDMX and MDM2 with p53. ALRN-6924 is currently being evaluated in clinical trials for the treatment of AML, advanced myelodysplastic syndrome (MDS) and peripheral T-cell lymphoma (PTCL) (NCT02909972; NCT02264613).

In some embodiments, one or more other therapeutic agent is an inhibitor of transforming growth factor-beta (TGF-beta or TGFß). Inhibitors of TGF-beta proteins being studied which may be used in the present invention include NIS793 (Novartis), an anti-TGF-beta antibody being tested in the clinic for treatment of various cancers, including breast, lung, hepatocellular, colorectal, pancreatic, prostate and renal cancer (NCT 02947165). In some embodiments, the inhibitor of TGF-beta proteins is fresolimumab (GC1008; Sanofi-Genzyme), which is being studied for melanoma (NCT00923169); renal cell carcinoma (NCT00356460); and non-small cell lung cancer (NCT02581787). Additionally, in some embodiments, the additional therapeutic agent is a TGF-beta trap, such as described in Connolly et al. (2012) Int'l J. Biological Sciences 8:964-978. One therapeutic compound currently in clinical trials for treatment of solid tumors is M7824 (Merck KgaA—formerly MSB0011459X), which is a bispecific, anti-PD-L1/TGFß trap compound (NCT02699515); and (NCT02517398). M7824 is comprised of a fully human IgG1 antibody against PD-L1 fused to the extracellular domain of human TGF-beta receptor II, which functions as a TGFß “trap.”

In some embodiments, one or more other therapeutic agent is selected from glembatumumab vedotin-monomethyl auristatin E (MMAE) (Celldex), an anti-glycoprotein NMB (gpNMB) antibody (CR011) linked to the cytotoxic MMAE, gpNMB is a protein overexpressed by multiple tumor types associated with cancer cells' ability to metastasize.

In some embodiments, one or more other therapeutic agent is an antiproliferative compound. Such antiproliferative compounds include, but are not limited to aromatase inhibitors; antiestrogens; topoisomerase I inhibitors; topoisomerase II inhibitors; microtubule active compounds; alkylating compounds; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; matrix metalloproteinase inhibitors; bisphosphonates; biological response modifiers; antiproliferative antibodies; heparanase inhibitors; inhibitors of Ras oncogenic isoforms; telomerase inhibitors; proteasome inhibitors; compounds used in the treatment of hematologic malignancies; compounds which target, decrease or inhibit the activity of Flt-3; Hsp90 inhibitors such as 17-AAG (17-allylaminogeldanamycin, NSC330507), 17-DMAG (17-dimethylaminoethylamino-17-demethoxy-geldanamycin, NSC707545), IPI-504, CNF1010, CNF2024, CNF1010 from Conforma Therapeutics; temozolomide (Temodal®); kinesin spindle protein inhibitors, such as SB715992 or SB743921 from GlaxoSmithKline, or pentamidine/chlorpromazine from CombinatoRx; MEK inhibitors such as ARRY142886 from Array BioPharma, AZd₆244 from AstraZeneca, PD181461 from Pfizer and leucovorin.

In some embodiments, the present invention provides a method of treating Alzheimer's disease comprising administering to a patient in need thereof a provided compound and one or more additional therapeutic agents selected from donepezil (Aricept®), rivastigmine (Excelon®), galantamine (Razadyne®), tacrine (Cognex®), and memantine (Namenda).

In some embodiments, one or more other therapeutic agent is a taxane compound, which causes disruption of microtubules, which are essential for cell division. In some embodiments, a taxane compound is selected from paclitaxel (Taxol®, Bristol-Myers Squibb), docetaxel (Taxotere®, Sanofi-Aventis; Doccfrez®, Sun Pharmaceutical), albumin-bound paclitaxel (Abraxane®; Abraxis/Celgene), cabazitaxel (Jevtana®, Sanofi-Aventis), and SID530 (SK Chemicals, Co.) (NCT00931008).

In some embodiments, one or more other therapeutic agent is a nucleoside inhibitor, or a therapeutic agent that interferes with normal DNA synthesis, protein synthesis, cell replication, or will otherwise inhibit rapidly proliferating cells.

In some embodiments, a nucleoside inhibitor is selected from trabectedin (guanidine alkylating agent, Yondelis®, Janssen Oncology), mechlorethamine (alkylating agent, Valchlor®, Aktelion Pharmaceuticals); vincristine (Oncovin®, Eli Lilly; Vincasar®, Teva Pharmaceuticals: Marqibo®. Talon Therapeutics); temozolomide (prodrug to alkylating agent 5-(3-methyltriazen-1-yl)-imidazole-4-carboxamide (MTIC) Temodar®, Merck); cytarabine injection (ara-C, antimetabolic cytidine analog, Pfizer); lomustine (alkylating agent, CeeNU®, Bristol-Myers Squibb; Gleostine®, NextSource Biotechnology); azacitidine (pyrimidine nucleoside analog of cytidine, Vidaza®, Celgene); omacetaxine mepesuccinate (cephalotaxine ester) (protein synthesis inhibitor, Synribo®; Teva Pharmaceuticals); asparaginase Erwinia chrysanthemi (enzyme for depletion of asparagine, Elspar®, Lundbeck; Erwinaze®, EUSA Pharma); eribulin mesylate (microtubule inhibitor, tubulin-based antimitotic, Halaven®, Eisai); cabazitaxel (microtubule inhibitor, tubulin-based antimitotic, Jevtana®, Sanofi-Aventis); capacetrine (thymidylate synthase inhibitor, Xeloda®, Genentech); bendamustine (bifunctional mechlorethamine derivative, believed to form interstrand DNA cross-links, Treanda®, Cephalon/Teva); ixabepilone (semi-synthetic analog of epothilone B, microtubule inhibitor, tubulin-based antimitotic. Ixempra®. Bristol-Myers Squibb); nelarabine (prodrug of deoxyguanosine analog, nucleoside metabolic inhibitor, Arranon®, Novartis); clorafabine (prodrug of ribonucleotide reductase inhibitor, competitive inhibitor of deoxycytidine, Clolar®, Sanofi-Aventis); and trifluridine and tipiracil (thymidine-based nucleoside analog and thymidine phosphorylase inhibitor, Lonsurf®, Taiho Oncology).

In some embodiments, one or more other therapeutic agent is a kinase inhibitor or VEGF-R antagonist. Approved VEGF inhibitors and kinase inhibitors useful in the present invention include: bevacizumab (Avastin®, Genentech/Roche) an anti-VEGF monoclonal antibody; ramucirmnab (Cyramza®, Eli Lilly), an anti-VEGFR-2 antibody and ziv-aflibercept, also known as VEGF Trap (Zaltrap®; Regeneron/Sanofi). VEGFR inhibitors, such as regorafenib (Stivarga®, Bayer); vandetanib (Caprelsa®, AstraZeneca); axitinib (Inlyta®, Pfizer); and lenvatinib (Lenvima®, Eisai); Raf inhibitors, such as sorafenib (Nexavar®. Bayer AG and Onyx); dabrafenib (Tafinlar®, Novartis); and vemurafenib (Zelboraf®, Genentech/Roche); MEK inhibitors, such as cobimetanib (Cotellic®, Exelexis/Genentech/Roche); trametinib (Mekinist®, Novartis); Bcr-Abl tyrosine kinase inhibitors, such as imatinib (Gleevec®, Novartis); nilotinib (Tasigna(®, Novartis); dasatinib (Sprycel®, BristolMyersSquibb); bosutinib (Bosulif®, Pfizer); and ponatinib (Inclusig®, Ariad Pharmaceuticals); Her2 and EGFR inhibitors, such as gefitinib (Iressa®, AstraZeneca); erlotinib (Tarceeva®, Genentech/Roche/Astellas); lapatinib (Tykerb®, Novartis); afatinib (Gilotrif®, Boehringer Ingelheim); osimertinib (targeting activated EGFR, Tagrisso®, AstraZeneca); and brigatinib (Alunbrig®, Ariad Pharmaceuticals); c-Met and VEGFR2 inhibitors, such as cabozanitib (Cometriq®, Exelexis); and multikinase inhibitors, such as sunitinib (Sutent®, Pfizer); pazopanib (Votrient®, Novartis); ALK inhibitors, such as crizotinib (Xalkori®, Pfizer); ceritinib (Zykadia®, Novartis); and alectinib (Alecenza®, Genentech/Roche); Bruton's tyrosine kinase inhibitors, such as ibrutinib (Imbruvica®, Pharmacyclics/Janssen); and Flt3 receptor inhibitors, such as midostaurin (Rydapt®, Novartis).

In some embodiments, the present invention provides a method of treating EGFR-mutant NSCLC in a patient in need thereof, comprising administering a compound of the present invention or a pharmaceutically acceptable salt thereof and one or more EGFR kinase inhibitors (e.g., gefitinib, erlotinib, lapatinib, afatinib, osimertinib, brigatinib, etc.).

In some embodiments, the present invention provides a method of treating EGFR-mutant NSCLC in a patient in need thereof, comprising administering a compound of the present invention or a pharmaceutically acceptable salt thereof and erlotinib.

Other kinase inhibitors and VEGF-R antagonists that are in development and may be used in the present invention include tivozanib (Aveo Pharmaceuticals); vatalanib (Bayer/Novartis); lucitanib (Clovis Oncology); dovitinib (TKI258, Novartis); Chiauanib (Chipscreen Biosciences); CEP-11981 (Cephalon); linifanib (Abbott Laboratories); neratinib (HKI-272, Puma Biotechnology); radotinib (Supect®, IY5511, Il-Yang Phannaceuticals, S. Korea); ruxolitinib (Jakafi®, Incyte Corporation); PTC299 (PTC Therapeutics); CP-547.632 (Pfizer); foretinib (Exelexis, GlaxoSmithKline); quizartinib (Daiichi Sankyo) and motesanib (Amgen/Takeda).

In another embodiment, the present invention provides a method of treating organ transplant rejection or graft vs. host disease comprising administering to a patient in need thereof a provided compound and one or more additional therapeutic agents selected from a steroid, cyclosporin, FK506, rapamycin, a hedgehog signaling inhibitor, a BTK inhibitor, a JAK/pan-JAK inhibitor, a TYK2 inhibitor, a PI3K inhibitor, and a SYK inhibitor.

In another embodiment, the present invention provides a method of treating or lessening the severity of a disease comprising administering to a patient in need thereof a provided compound and a BTK inhibitor, wherein the disease is selected from inflammatory bowel disease, arthritis, systemic lupus erythematosus (SLE), vasculitis, idiopathic thrombocytopenic purpura (ITP), rheumatoid arthritis, psoriatic arthritis, osteoarthritis, Still's disease, juvenile arthritis, diabetes, myasthenia gravis. Hashimoto's thyroiditis, Ord's thyroiditis, Graves' disease, autoimmune thyroiditis, Sjogren's syndrome, multiple sclerosis, systemic sclerosis, Lyme neuroborreliosis, Guillain-Barre syndrome, acute disseminated encephalomyelitis, Addison's disease, opsoclonus-myoclonus syndrome, ankylosing spondylosis, antiphospholipid antibody syndrome, aplastic anemia, autoimmune hepatitis, autoimmune gastritis, pernicious anemia, celiac disease, Goodpasture's syndrome, idiopathic thrombocytopenic purpura, optic neuritis, sclerodenna, primary biliary cirrhosis, Reiter's syndrome, Takayasu's arteritis, temporal arteritis, warm autoimmune hemolytic anemia, Wegener's granulomatosis, psoriasis, alopecia universalis, Beheet's disease, chronic fatigue, dysautonomia, membranous glomerulonephropathy, endometriosis, interstitial cystitis, pemphigus vulgaris, bullous pemphigoid, neuromyotonia, scleroderma, vulvodynia, a hyperproliferative disease, rejection of transplanted organs or tissues, Acquired Immunodeficiency Syndrome (AIDS, also known as HIV), type 1 diabetes, graft versus host disease, transplantation, transfusion, anaphylaxis, allergies (e.g., allergies to plant pollens, latex, drugs, foods, insect poisons, animal hair, animal dander, dust mites, or cockroach calyx), type I hypersensitivity, allergic conjunctivitis, allergic rhinitis, asthma, appendicitis, atopic dermatitis, asthma, allergy, blepharitis, bronchiolitis, bronchitis, bursitis, cervicitis, cholangitis, cholecystitis, chronic graft rejection, colitis, conjunctivitis, Crohn's disease, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, uveitis, vaginitis, vasculitis, or vulvitis, B-cell proliferative disorder, e.g., diffuse large B cell lymphoma, follicular lymphoma, chronic lymphocytic lymphoma, chronic lymphocytic leukemia, acute lymphocytic leukemia, B-cell prolymphocytic leukemia, lymphoplasmacytic lymphoma/Waldenstrom macroglobulinemia, splenic marginal zone lymphoma, multiple myeloma (also known as plasma cell myeloma), non-Hodgkin's lymphoma, Hodgkin's lymphoma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, Burkitt lymphoma/leukemia, or lvmphomatoid granulomatosis, breast cancer, prostate cancer, or cancer of the mast cells (e.g., mastocytoma, mast cell leukemia, mast cell sarcoma, systemic mastocytosis), bone cancer, colorectal cancer, pancreatic cancer, diseases of the bone and joints leading to joint inflammation and pain, cartilage and/or bone destruction, as well as bone regrowth and fusion, including, without limitation, rheumatoid arthritis, seronegative spondyloarthropathies (including ankylosing spondylitis, psoriatic arthritis and Reiter's disease), Behcet's disease, Sjogren's syndrome, systemic sclerosis, osteoporosis, bone cancer, bone metastasis, a thromboembolic disorder, (e.g., myocardial infarct, angina pectoris, reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, deep venous thrombosis), inflammatory pelvic disease, urethritis, skin sunbun, sinusitis, pneumonitis, encephalitis, meningitis, myocarditis, nephritis, osteomyelitis, myositis, hepatitis, idiopathic autoimmune hepatitis, gastritis, enteritis, dermatitis, gingivitis, appendicitis, pancreatitis, cholocystitus, agammaglobulinemia, psoriasis, allergy, Crohn's disease, irritable bowel syndrome, ulcerative colitis, Sjogren's disease, tissue graft rejection, hyperacute rejection of transplanted organs, asthma, allergic rhinitis, chronic obstructive pulmonary disease (COPD), autoimmune polyglandular disease (also known as autoimmune polyglandular syndrome), autoimmune alopecia, pernicious anemia, glomerulonephritis, dermatomyositis, multiple sclerosis, scleroderma, vasculitis, autoimmune hemolytic and thrombocytopenic states, Goodpasture's syndrome, atherosclerosis, Addison's disease, Parkinson's disease, Alzheimer's disease, diabetes, septic shock, systemic lupus erythematosus (SLE), rheumatoid arthritis, psoriatic arthritis, juvenile arthritis, osteoarthritis, chronic idiopathic thrombocytopenic purpura, Waldenstrom macroglobulinemia, myasthenia gravis, Hashimoto's thyroiditis, degenerative joint disease, vitiligo, autoimmune hypopituitarism, Guillain-Barre syndrome, Behcet's disease, scleroderma, mycosis fungoides, acute inflammatory responses (such as acute respiratory distress syndrome and ischemia/reperfusion injury), and Graves' disease.

In another embodiment, the present invention provides a method of treating or lessening the severity of a disease comprising administering to a patient in need thereof a provided compound and a PI3K inhibitor, wherein the disease is selected from a cancer, a neurodegenerative disorder, an angiogenic disorder, a viral disease, an autoimmune disease, an inflammatory disorder, a hormone-related disease, conditions associated with organ transplantation, immunodeficiency disorders, a destructive or overgrowing bone disorder, a proliferative disorder, an infectious disease, a condition associated with cell death, thrombin-induced platelet aggregation, chronic myelogenous leukemia (CML), chronic lymphocytic leukemia (CLL), liver disease, pathologic immune conditions involving T cell activation, a cardiovascular disorder, and a CNS disorder.

In another embodiment, the present invention provides a method of treating or lessening the severity of a disease comprising administering to a patient in need thereof a provided compound and a PI3K inhibitor, wherein the disease is selected from benign or malignant tumor, carcinoma or solid tumor of the brain, kidney (e.g., renal cell carcinoma (RCC)), liver, adrenal gland, bladder, breast, stomach, gastric tumors, ovaries, colon, rectum, prostate, pancreas, lung, vagina, endometrium, cervix, testis, genitourinary tract, esophagus, larynx, skin, bone or thyroid, sarcoma, glioblastomas, neuroblastomas, multiple myeloma or gastrointestinal cancer, especially colon carcinoma or colorectal adenoma or a tumor of the neck and head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, non-small-cell lung carcinoma, lymphomas, (including, for example, non-Hodgkin's Lymphoma (NHL) and Hodgkin's lymphoma (also termed Hodgkin's or Hodgkin's disease)), a mammary carcinoma, follicular carcinoma, undifferentiated carcinoma, papillary carcinoma, seminoma, melanoma, or a leukemia, diseases include Cowden syndrome, Lhermitte-Dudos disease and Bannayan-Zonana syndrome, or diseases in which the PI3K/PKB pathway is aberrantly activated, asthma of whatever type or genesis including both intrinsic (non-allergic) asthma and extrinsic (allergic) asthma, mild asthma, moderate asthma, severe asthma, bronchitic asthma, exercise-induced asthma, occupational asthma and asthma induced or exacerbated following bacterial or viral infection, acute lung injury (ALI), adult/acute respiratory distress syndrome (ARDS), chronic obstructive pulmonary, airways or lung disease (COPD, COAD or COLD), including chronic bronchitis or dyspnea associated therewith, emphysema, as well as exacerbation of airways hyperreactivity consequent to other drug therapy, in particular other inhaled drug therapy, bronchitis of whatever type or genesis including, but not limited to, acute, arachidic, catarrhal, croupus, chronic or phthinoid bronchitis, pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis, Loffler's syndrome, eosinophilic, pneumonia, parasitic (in particular metazoan) infestation (including tropical eosinophilia), bronchopulmonary aspergillosis, polyarteritis nodosa (including Churg-Strauss syndrome), eosinophilic granuloma and eosinophil-related disorders affecting the airways occasioned by drug-reaction, psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, pemphisus, epidermolysis bullosa acquisita, conjunctivitis, keratoconjunctivitis sicca, and vernal conjunctivitis, diseases affecting the nose including allergic rhinitis, and inflammatory disease in which autoimmune reactions are implicated or having an autoimmune component or etiology, including autoimmune hematological disorders (e.g. hemolytic anemia, aplastic anemia, pure red cell anemia and idiopathic thrombocytopenia), systemic lupus erythematosus, rheumatoid arthritis, polychondritis, scleredoma, Wegener granulamatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), endocrine opthalmopathy, Grave's disease, sarcoidosis, alveolitis, chronic hypersensitivity pneumonitis, multiple sclerosis, primary biliary cirrhosis, uveitis (anterior and posterior), keratoconjunctivitis sicca and vernal keratoconjunctivitis, interstitial lung disease or fibrosis, psoriatic arthritis and glomerulonephritis (with and without nephrotic syndrome, e.g. including idiopathic nephrotic syndrome or minal change nephropathy, restenosis, cardiomegaly, atherosclerosis, myocardial infarction, ischemic stroke and congestive heart failure, Alzheimer's disease, Parkinson's disease, amyotrophic lateral sclerosis, Huntington's disease, and cerebral ischemia, and neurodegenerative disease caused by traumatic injury, glutamate neurotoxicity and hypoxia.

In some embodiments, one or more other therapeutic agent is a phosphatidylinositol 3 kinase (PI3K) inhibitor. In some embodiments, a PI3K inhibitor is selected from idelalisib (Zydelig®, Gilead), alpelisib (BYL719, Novartis), taselisib (GDC-0032, Genentech/Roche); pictilisib (GDC-0941, Genentech/Roche); copanlisib (BAY806946, Bayer); duvelisib (formerly IPI-145, Infinity Pharmaceuticals); PQR309 (Piqur Therapeutics, Switzerland); and TGR1202 (formerly RP5230, TG Therapeutics).

Depending upon the particular condition, or disease, to be treated, additional therapeutic agents that are normally administered to treat that condition, may also be present in the compositions of this invention. As used herein, additional therapeutic agents that are normally administered to treat a particular disease, or condition, are known as “appropriate for the disease, or condition, being treated.”

A compound of the current invention may also be used to advantage in combination with other antiproliferative compounds. Such antiproliferative compounds include, but are not limited to aromatase inhibitors; antiestrogens; topoisomerase I inhibitors; topoisomerase II inhibitors; microtubule active compounds; alkylating compounds; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; matrix metalloproteinase inhibitors; bisphosphonates; biological response modifiers; antiproliferative antibodies; heparanase inhibitors; inhibitors of Ras oncogenic isoforms; telomerase inhibitors; proteasome inhibitors; compounds used in the treatment of hematologic malignancies; compounds which target, decrease or inhibit the activity of Flt-3; Hsp90 inhibitors such as 17-AAG (17-allylaminogeldanamycin, NSC330507), 17-DMAG (17-dimethylaminoethylamino-17-demethoxy-geldanamycin, NSC707545), IPI-504, CNF1010, CNF2024. CNF1010 from Conforma Therapeutics; temozolomide (Temodal®); kinesin spindle protein inhibitors, such as SB715992 or SB743921 from GlaxoSmithKline, or pentamidine/chlorpromazine from CombinatoRx; MEK inhibitors such as ARRY142886 from Array BioPharma, AZD6244 from AstraZeneca, PD181461 from Pfizer and leucovorin.

The term “aromatase inhibitor” as used herein relates to a compound which inhibits estrogen production, for instance, the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole. Exemestane is marketed under the trade name Aromasin™. Formestane is marketed under the trade name Lentaron™. Fadrozole is marketed under the trade name Afema™. Anastrozole is marketed under the trade name Arimidex™. Letrozole is marketed under the trade names Femara™ or Femar™. Aminoglutethimide is marketed under the trade name Orimeten™. A combination of the invention comprising a chemotherapeutic agent which is an aromatase inhibitor is particularly useful for the treatment of hormone receptor positive tumors, such as breast tumors.

In some embodiments, one or more other therapeutic agent is an mTOR inhibitor, which inhibits cell proliferation, angiogenesis and glucose uptake. In some embodiments, an mTOR inhibitor is everolimus (Afinitor®, Novartis); temsirolimus (Torisel®, Pfizer); and sirolimus (Rapamune®, Pfizer).

In some embodiments, one or more other therapeutic agent is an aromatase inhibitor. In some embodiments, an aromatase inhibitor is selected from exemestane (Aromasin®, Pfizer); anastazole (Arimidex®, AstraZeneca) and letrozole (Femara®, Novartis).

The term “antiestrogen” as used herein relates to a compound which antagonizes the effect of estrogens at the estrogen receptor level. The term includes, but is not limited to tamoxifen, fulvestrant, raloxifene and raloxifene hydrochloride. Tamoxifen is marketed under the trade name Nolvadex™. Raloxifene hydrochloride is marketed under the trade name Evista™. Fulvestrant can be administered under the trade name Faslodex™. A combination of the invention comprising a chemotherapeutic agent which is an antiestrogen is particularly useful for the treatment of estrogen receptor positive tumors, such as breast tumors.

The term “anti-androgen” as used herein relates to any substance which is capable of inhibiting the biological effects of androgenic hormones and includes, but is not limited to, bicalutamide (Casodex™). The term “gonadorelin agonist” as used herein includes, but is not limited to abarelix, goserelin and goserelin acetate. Goserelin can be administered under the trade name Zoladex™.

The term “topoisomerase I inhibitor” as used herein includes, but is not limited to topotecan, gimatecan, irinotecan, camptothecian and its analogues, 9-nitrocamptothecin and the macromolecular camptothecin conjugate PNU-166148. Irinotecan can be administered, e.g. in the form as it is marketed, e.g. under the trademark Camptosar™. Topotecan is marketed under the trade name Hycamptin™.

The term “topoisomerase II inhibitor” as used herein includes, but is not limited to the anthracyclines such as doxorubicin (including liposomal formulation, such as Caelyx™), daunorubicin, epirubicin, idarubicin and nemorubicin, the anthraquinones mitoxantrone and losoxantrone, and the podophillotoxines etoposide and teniposide. Etoposide is marketed under the trade name Etopophos™ Teniposide is marketed under the trade name VM 26-Bristol Doxorubicin is marketed under the trade name Acriblastin™ or Adriamycin™. Epirubicin is marketed under the trade name Farmorubicin™. Idarubicin is marketed, under the trade name Zavedos™. Mitoxantrone is marketed under the trade name Novantron.

The term “microtubule active agent” relates to microtubule stabilizing, microtubule destabilizing compounds and microtublin polymerization inhibitors including, but not limited to taxanes, such as paclitaxel and docetaxel; vinca alkaloids, such as vinblastine or vinblastine sulfate, vincristine or vincristine sulfate, and vinorelbine; discodemiolides; cochicine and epothilones and derivatives thereof. Paclitaxel is marketed under the trade name Taxol™. Docetaxel is marketed under the trade name Taxotere™. Vinblastine sulfate is marketed under the trade name Vinblastin R. P™. Vincristine sulfate is marketed under the trade name Farmistin™.

The term “alkylating agent” as used herein includes, but is not limited to, cyclophosphamide, ifosfamide, melphalan or nitrosourea (BCNU or Gliadel). Cyclophosphamide is marketed under the trade name Cyclostin™. Ifosfamide is marketed under the trade name Holoxan™.

The term “histone deacetylase inhibitors” or “HDAC inhibitors” relates to compounds which inhibit the histone deacetylase and which possess antiproliferative activity. This includes, but is not limited to, suberoylanilide hydroxamic acid (SAHA).

The term “antineoplastic antimetabolite” includes, but is not limited to, 5-fluorouracil or 5-FU, capecitabine, gemcitabine, DNA demethylating compounds, such as 5-azacytidine and decitabine, methotrexate and edatrexate, and folic acid antagonists such as pemetrexed. Capecitabine is marketed under the trade name Xeloda™. Gemcitabine is marketed under the trade name Gemzar™.

The term “platin compound” as used herein includes, but is not limited to, carboplatin, cis-platin, cisplatinum and oxaliplatin. Carboplatin can be administered, e.g., in the form as it is marketed, e.g. under the trademark Carboplat™. Oxaliplatin can be administered, e.g., in the form as it is marketed. e.g. under the trademark Eloxatin™.

The term “Bcl-2 inhibitor” as used herein includes, but is not limited to compounds having inhibitory activity against B-cell lymphoma 2 protein (Bcl-2), including but not limited to ABT-199, ABT-731, ABT-737, apogossypol, Ascenta's pan-Bcl-2 inhibitors, curcumin (and analogs thereof), dual Bel-2/Bcl-xL inhibitors (Infinity Pharmaceuticals/Novartis Pharmaceuticals), Genasense (G3139), HA14-1 (and analogs thereof; see WO2008118802), navitoclax (and analogs thereof, see U.S. Pat. No. 7,390,799). NH-1 (Shenayng Pharmaceutical University), obatoclax (and analogs thereof, see WO2004106328), S-001 (Gloria Pharmaceuticals), TW series compounds (Univ. of Michigan), and venetoclax. In some embodiments the Bcl-2 inhibitor is a small molecule therapeutic. In some embodiments the Bcl-2 inhibitor is a peptidomimetic.

The term “compounds targeting/decreasing a protein or lipid kinase activity; or a protein or lipid phosphatase activity; or further anti-angiogenic compounds” as used herein includes, but is not limited to, protein tyrosine kinase and/or serine and/or threonine kinase inhibitors or lipid kinase inhibitors, such as a) compounds targeting, decreasing or inhibiting the activity of the platelet-derived growth factor-receptors (PDGFR), such as compounds which target, decrease or inhibit the activity of PDGFR, especially compounds which inhibit the PDGF receptor, such as an N-phenyl-2-pyrimidine-amine derivative, such as imatinib, SU101, SU6668 and GFB-111; b) compounds targeting, decreasing or inhibiting the activity of the fibroblast growth factor-receptors (FGFR); c) compounds targeting, decreasing or inhibiting the activity of the insulin-like growth factor receptor I (IGF-IR), such as compounds which target, decrease or inhibit the activity of IGF-IR, especially compounds which inhibit the kinase activity of IGF-I receptor, or antibodies that target the extracellular domain of IGF-I receptor or its growth factors; d) compounds targeting, decreasing or inhibiting the activity of the Trk receptor tyrosine kinase family, or ephrin B4 inhibitors; e) compounds targeting, decreasing or inhibiting the activity of the AxI receptor tyrosine kinase family; f) compounds targeting, decreasing or inhibiting the activity of the Ret receptor tyrosine kinase; g) compounds targeting, decreasing or inhibiting the activity of the Kit/SCFR receptor tyrosine kinase, such as imatinib; h) compounds targeting, decreasing or inhibiting the activity of the C-kit receptor tyrosine kinases, which are part of the PDGFR family, such as compounds which target, decrease or inhibit the activity of the c-Kit receptor tyrosine kinase family, especially compounds which inhibit the c-Kit receptor, such as imatinib; i) compounds targeting, decreasing or inhibiting the activity of members of the c-Abl family, their gene-fusion products (e.g. BCR-Abl kinase) and mutants, such as compounds which target decrease or inhibit the activity of c-Abl family members and their gene fusion products, such as an N-phenyl-2-pyrimidine-amine derivative, such as imatinib or nilotinib (AMN107); PD180970; AG957; NSC 680410; PD173955 from ParkeDavis; or dasatinib (BMS-354825); j) compounds targeting, decreasing or inhibiting the activity of members of the protein kinase C (PKC) and Raf family of serine/threonine kinases, members of the MEK, SRC, JAK/pan-JAK, FAK, PDK1, PKB/Akt, Ras/MAPK, PI3K, SYK, TYK2, BTK and TEC family, and/or members of the cyclin-dependent kinase family (CDK) including staurosporine derivatives, such as midostaurin; examples of further compounds include UCN-01, safingol, BAY 43-9006, Bryostatin 1, Perifosine; llmofosine; RO 318220 and RO 320432; GO 6976; lsis 3521; LY333531/LY379196; isochinoline compounds; FTIs; PD184352 or QAN697 (a PI3K inhibitor) or AT7519 (CDK inhibitor); k) compounds targeting, decreasing or inhibiting the activity of protein-tyrosine kinase inhibitors, such as compounds which target, decrease or inhibit the activity of protein-tyrosine kinase inhibitors include imatinib mesylate (Gleevec™) or tyrphostin such as Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin); 1) compounds targeting, decreasing or inhibiting the activity of the epidermal growth factor family of receptor tyrosine kinases (EGFR₁ ErbB2, ErbB3, ErbB4 as homo- or heterodimers) and their mutants, such as compounds which target, decrease or inhibit the activity of the epidermal growth factor receptor family are especially compounds, proteins or antibodies which inhibit members of the EGF receptor tyrosine kinase family, such as EGF receptor, ErbB2, ErbB3 and ErbB4 or bind to EGF or EGF related ligands, CP 358774. ZD 1839, ZM 105180; trastuzumab (Herceptin™), cetuximab (Erbitux™), Iressa, Tarceva, OSI-774, Cl-1033. EKB-569, GW-2016, E1.1, E2.4, E2.5, E6.2, E6.4, E2.11, E6.3 or E7.6.3, and 7H-pyrrolo-[2,3-d]pyrimidine derivatives; m) compounds targeting, decreasing or inhibiting the activity of the c-Met receptor, such as compounds which target, decrease or inhibit the activity of c-Met, especially compounds which inhibit the kinase activity of c-Met receptor, or antibodies that target the extracellular domain of c-Met or bind to HGF, n) compounds targeting, decreasing or inhibiting the kinase activity of one or more JAK family members (JAK1/JAK2/JAK3/TYK2 and/or pan-JAK), including but not limited to PRT-062070, SB-1578, baricitinib, pacritinib, momelotinib, VX-509, AZD-1480, TG-101348, tofacitinib, and ruxolitinib; o) compounds targeting, decreasing or inhibiting the kinase activity of PI3 kinase (PI3K) including but not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib; and; and q) compounds targeting, decreasing or inhibiting the signaling effects of hedgehog protein (Hh) or smoothened receptor (SMO) pathways, including but not limited to cyclopamine, vismodegib, itraconazole, erismodegib, and IPI-926 (saridegib).

Compounds which target, decrease or inhibit the activity of a protein or lipid phosphatase are e.g. inhibitors of phosphatase 1, phosphatase 2A, or CDC25, such as okadaic acid or a derivative thereof.

In some embodiments, one or more other therapeutic agent is a growth factor antagonist, such as an antagonist of platelet-derived growth factor (PDGF), or epidermal growth factor (EGF) or its receptor (EGFR). Approved PDGF antagonists which may be used in the present invention include olaratumab (Lartruvo®; Eli Lilly). Approved EGFR antagonists which may be used in the present invention include cetuximab (Erbitux®, Eli Lilly); necitumumab (Portrazza®, Eli Lilly), panitumumab (Vectibix®, Amgen); and osimertinib (targeting activated EGFR, Tagrisso®, AstraZeneca).

The term “PI3K inhibitor” as used herein includes, but is not limited to compounds having inhibitory activity against one or more enzymes in the phosphatidylinositol-3-kinase family, including, but not limited to PI3Kα, PI3Kγ, PI3Kδ, PI3Kβ, PI3K-C2α, PI3K-C2β, PI3K-C2γ, Vps34, p110-α, p110-β, p110-γ, p110-δ, p85-α, p85-β, p55-γ, p150, p101, and p87. Examples of PI3K inhibitors useful in this invention include but are not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib.

The term “BTK inhibitor” as used herein includes, but is not limited to compounds having inhibitory activity against Bruton's Tyrosine Kinase (BTK), including, but not limited to AVL-292 and ibrutinib.

The term “SYK inhibitor” as used herein includes, but is not limited to compounds having inhibitory activity against spleen tyrosine kinase (SYK), including but not limited to PRT-062070, R-343, R-333, Excellair, PRT-062607, and fostamatinib

Further examples of BTK inhibitory compounds, and conditions treatable by such compounds in combination with compounds of this invention can be found in WO2008039218 and WO2011090760, the entirety of which are incorporated herein by reference.

Further examples of SYK inhibitory compounds, and conditions treatable by such compounds in combination with compounds of this invention can be found in WO2003063794, WO2005007623, and WO2006078846, the entirety of which are incorporated herein by reference.

Further examples of PI3K inhibitory compounds, and conditions treatable by such compounds in combination with compounds of this invention can be found in WO2004019973, WO2004089925, WO2007016176, U.S. Pat. No. 8,138,347, WO2002088112, WO2007084786, WO2007129161, WO2006122806, WO2005113554, and WO2007044729 the entirety of which are incorporated herein by reference.

Further examples of JAK inhibitory compounds, and conditions treatable by such compounds in combination with compounds of this invention can be found in WO2009114512, WO2008109943, WO2007053452, WO2000142246, and WO2007070514, the entirety of which are incorporated herein by reference.

Further anti-angiogenic compounds include compounds having another mechanism for their activity, e.g. unrelated to protein or lipid kinase inhibition e.g. thalidomide (Thalomid™) and TNP-470.

Examples of proteasome inhibitors useful for use in combination with compounds of the invention include, but are not limited to bortezomib, disulfiram, epigallocatechin-3-gallate (EGCG), salinosporamide A, carfilzomib, ONX-0912, CEP-18770, and MLN9708.

Compounds which target, decrease or inhibit the activity of a protein or lipid phosphatase are e.g. inhibitors of phosphatase 1, phosphatase 2A, or CDC25, such as okadaic acid or a derivative thereof.

Compounds which induce cell differentiation processes include, but are not limited to, retinoic acid, α- γ- or δ-tocopherol or α- γ- or δ-tocotrienol.

The term cyclooxygenase inhibitor as used herein includes, but is not limited to, Cox-2 inhibitors, 5-alkyl substituted 2-arylaminophenylacetic acid and derivatives, such as celecoxib (Celebrex™), rofecoxib (Vioxx™), etoricoxib, valdecoxib or a 5-alkyl-2-arylaminophenylacetic acid, such as 5-methlv-2-(2′-chloro-6′-fluoroanilino)phenyl acetic acid, lumiracoxib.

The term “bisphosphonates” as used herein includes, but is not limited to, etridonic, clodronic, tiludronic, pamidronic, alendronic, ibandronic, risedronic and zoledronic acid. Etridonic acid is marketed under the trade name Didronel™. Clodronic acid is marketed under the trade name Bonefos™. Tiludronic acid is marketed under the trade name Skelid™. Pamidronic acid is marketed under the trade name Aredia™. Alendronic acid is marketed under the trade name Fosamax™. Ibandronic acid is marketed under the trade name Bondranat™. Risedronic acid is marketed under the trade name Actonel™. Zoledronic acid is marketed under the trade name Zometa™. The term “mTOR inhibitors” relates to compounds which inhibit the mammalian target of rapamycin (mTOR) and which possess antiproliferative activity such as sirolimus (Rapamune®), everolimus (Certican™), CCI-779 and ABT578.

The term “heparanase inhibitor” as used herein refers to compounds which target, decrease or inhibit heparin sulfate degradation. The term includes, but is not limited to. PI-88. The term “biological response modifier” as used herein refers to a lymphokine or interferons.

The term “inhibitor of Ras oncogenic isoforms”, such as H-Ras, K-Ras, or N-Ras, as used herein refers to compounds which target, decrease or inhibit the oncogenic activity of Ras; for example, a “farnesyl transferase inhibitor” such as L-744832, DK8G557 or R115777 (Zarnestra™). The term “telomerase inhibitor” as used herein refers to compounds which target, decrease or inhibit the activity of telomerase. Compounds which target, decrease or inhibit the activity of telomerase are especially compounds which inhibit the telomerase receptor, such as telomestatin.

The term “methionine aminopeptidase inhibitor” as used herein refers to compounds which target, decrease or inhibit the activity of methionine aminopeptidase. Compounds which target, decrease or inhibit the activity of methionine aminopeptidase include, but are not limited to, bengamide or a derivative thereof.

The term “proteasome inhibitor” as used herein refers to compounds which target, decrease or inhibit the activity of the proteasome. Compounds which target, decrease or inhibit the activity of the proteasome include, but are not limited to, Bortezomib (Velcade™), carfilzomib (Kyprolis®, Amgen); and ixazomib (Ninlaro®, Takeda), and MLN 341.

The term “matrix metalloproteinase inhibitor” or (“MMP” inhibitor) as used herein includes, but is not limited to, collagen peptidomimetic and nonpeptidomimetic inhibitors, tetracycline derivatives, e.g. hydroxamate peptidomimetic inhibitor batimastat and its orally bioavailable analogue marimastat (BB-2516), prinomastat (AG3340), metastat (NSC 683551) BMS-279251, BAY 12-9566, TAA211, MMI270B or AAJ996.

The term “compounds used in the treatment of hematologic malignancies” as used herein includes, but is not limited to, FMS-like tyrosine kinase inhibitors, which are compounds targeting, decreasing or inhibiting the activity of FMS-like tyrosine kinase receptors (Flt-3R); interferon, 1-β-D-arabinofuransylcytosine (ara-c) and bisulfan; and ALK inhibitors, which are compounds which target, decrease or inhibit anaplastic lymphoma kinase.

Compounds which target, decrease or inhibit the activity of FMS-like tyrosine kinase receptors (Flt-3R) are especially compounds, proteins or antibodies which inhibit members of the Flt-3R receptor kinase family, such as PKC412, midostaurin, a staurosporine derivative, SU11248 and MLN518.

The term “HSP90 inhibitors” as used herein includes, but is not limited to, compounds targeting, decreasing or inhibiting the intrinsic ATPase activity of HSP90; degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins or antibodies which inhibit the ATPase activity of HSP90, such as 17-allylamino,17-demethoxygeldanamycin (17AAG), a geldanamycin derivative; other geldanamycin related compounds; radicicol and HDAC inhibitors.

The term “antiproliferative antibodies” as used herein includes, but is not limited to, trastuzumab (Herceptin™), Trastuzumab-DM1, erbitux, bevacizumab (Avastin™), rituximab (Rituxan®), PR064553 (anti-CD40) and 2C4 Antibody. By antibodies is meant intact monoclonal antibodies, polyclonal antibodies, multispecific antibodies formed from at least 2 intact antibodies, and antibodies fragments so long as they exhibit the desired biological activity.

For the treatment of acute mycloid leukemia (AML), compounds of the current invention can be used in combination with standard leukemia therapies, especially in combination with therapies used for the treatment of AML. In particular, compounds of the current invention can be administered in combination with, for example, farnesyl transferase inhibitors and/or other drugs useful for the treatment of AML, such as Daunorubicin, Adriamycin, Ara-C, VP-16, Teniposide, Mitoxantrone, Idarubicin, Carboplatinum and PKC412.

Other anti-leukemic compounds include, for example, Ara-C, a pyrimidine analog, which is the 2′-alpha-hydroxy ribose (arabinoside) derivative of deoxycytidine. Also included is the purine analog of hypoxanthine, 6-mercaptopurine (6-MP) and fludarabine phosphate. Compounds which target, decrease or inhibit activity of histone deacetylase (HDAC) inhibitors such as sodium butyrate and suberoylanilide hydroxamic acid (SAHA) inhibit the activity of the enzymes known as histone deacetylases. Specific HDAC inhibitors include MS275, SAHA, FK228 (formerly FR901228), Trichostatin A and compounds disclosed in U.S. Pat. No. 6,552,065 including, but not limited to, N-hydroxy-3-[4-[[[2-(2-methyl-1H-indol-3-yl)-ethyl]-amino]methyl]phenyl]-2E-2-propenamide, or a pharmaceutically acceptable salt thereof and N-hydroxy-3-[4-[(2-hydroxyethyl){2-(1H-indol-3-yl)ethyl]-amino]methyl]phenyl]-2E-2-propenamide, or a pharmaceutically acceptable salt thereof, especially the lactate salt. Somatostatin receptor antagonists as used herein refer to compounds which target, treat or inhibit the somatostatin receptor such as octreotide, and SOM230. Tumor cell damaging approaches refer to approaches such as ionizing radiation. The term “ionizing radiation” referred to above and hereinafter means ionizing radiation that occurs as either electromagnetic rays (such as X-rays and gamma rays) or particles (such as alpha and beta particles). Ionizing radiation is provided in, but not limited to, radiation therapy and is known in the art. See Hellman, Principles of Radiation Therapy, Cancer, in Principles and Practice of Oncology, Devita et al., Eds., 4^{th} Edition, Vol. 1, pp. 248-275 (1993).

Also included are EDG binders and ribonucleotide reductase inhibitors. The term “EDG binders” as used herein refers to a class of immunosuppressants that modulates lymphocyte recirculation, such as FTY720. The term “ribonucleotide reductase inhibitors” refers to pyrimidine or purine nucleoside analogs including, but not limited to, fludarabine and/or cytosine arabinoside (ara-C), 6-thioguanine, 5-fluorouracil, cladribine, 6-mercaptopurine (especially in combination with ara-C against ALL) and/or pentostatin. Ribonucleotide reductase inhibitors are especially hydroxyurea or 2-hydroxy-1H-isoindole-1 ,3-dione derivatives.

Also included are in particular those compounds, proteins or monoclonal antibodies of VEGF such as 1-(4-chloroanilino)-4-(4-pyridylmethyl)phthalazine or a pharmaceutically acceptable salt thereof, 1-(4-chloroanilino)-4-(4-pyridylmethyl)phthalazine succinate; Angiostatin™; Endostatin™ anthranilic acid amides; ZD4190; ZD6474; SU5416; SU6668; bevacizumab; or anti-VEGF antibodies or anti-VEGF receptor antibodies, such as rhuMAb and RHUFab, VEGF aptamer such as Macugon; FLT-4 inhibitors, FLT-3 inhibitors, VEGFR-2 IgGI antibody, Angiozyme (RPI 4610) and Bevacizumab (Avastin™).

Photodynamic therapy as used herein refers to therapy which uses certain chemicals known as photosensitizing compounds to treat or prevent cancers. Examples of photodynamic therapy include treatment with compounds, such as Visudyne™ and porfimer sodium.

Angiostatic steroids as used herein refers to compounds which block or inhibit angiogenesis, such as, e.g., anecortave, triamcinolone, hydrocortisone, 11-α-epihydrocotisol, cortexolone, 17α-hydroxyprogesterone, corticosterone, desoxycorticosterone, testosterone, estrone and dexamethasone.

Implants containing corticosteroids refers to compounds, such as fluocinolonc and dexamethasone.

Other chemotherapeutic compounds include, but are not limited to, plant alkaloids, hormonal compounds and antagonists; biological response modifiers, preferably lymphokines or interferons; antisense oligonucleotides or oligonucleotide derivatives; shRNA or siRNA; or miscellaneous compounds or compounds with other or unknown mechanism of action.

The compounds of the invention are also useful as co-therapeutic compounds for use in combination with other drug substances such as anti-inflammatory, bronchodilatory or antihistamine drug substances, particularly in the treatment of obstructive or inflammatory airways diseases such as those mentioned hereinbefore, for example as potentiators of therapeutic activity of such drugs or as a means of reducing required dosaging or potential side effects of such drugs. A compound of the invention may be mixed with the other drug substance in a fixed pharmaceutical composition or it may be administered separately, before, simultaneously with or after the other drug substance. Accordingly the invention includes a combination of a compound of the invention as hereinbefore described with an anti-inflammatory, bronchodilatory, antihistamine or anti-tussive drug substance, said compound of the invention and said drug substance being in the same or different pharmaceutical composition.

Suitable anti-inflammatory drugs include steroids, in particular glucocorticosteroids such as budesonide, beclamethasone dipropionate, fluticasone propionate, ciclesonide or mometasone furoate; non-steroidal glucocorticoid receptor agonists; LTB4 antagonists such LY293111, CGS025019C, CP-195543, SC-53228, BIIL 284, ONO 4057, SB 209247; LTD4 antagonists such as montelukast and zafirlukast; PDE4 inhibitors such cilomilast (Ariflo® GlaxoSmithKline), Roflumilast (Byk Gulden), V-11294A (Napp), BAY19-8004 (Bayer), SCH-351591 (Schering- Plough), Arofylline (Almirall Prodesfarma), PD189659/PD168787 (Parke-Davis), AWD-12-281 (Asta Medica), CDC—801 (Celgene), SeICID™ CC-10004 (Celgene), VM554/UM565 (Vemalis), T-440 (Tanabe), KW-4490 (Kyowa Hakko Kogyo); A2a agonists; A2b antagonists; and beta-2 adrenoceptor agonists such as albuterol (salbutamol), metaproterenol, terbutaline, salmeterol fenoterol, procaterol, and especially, formoterol and pharmaceutically acceptable salts thereof. Suitable bronchodilatory drugs include anticholinergic or antimuscarinic compounds, in particular ipratropium bromide, oxitropium bromide, tiotropium salts and CHF 4226 (Chiesi), and glycopyrrolate.

Suitable antihistamine drug substances include cetirizine hydrochloride, acetaminophen, clemastine fumarate, promethazine, loratidine, desloratidine, diphenhydramine and fexofenadine hydrochloride, activastine, astemizole, azelastine, ebastine, epinastine, mizolastine and tefenadine.

Other useful combinations of compounds of the invention with anti-inflammatory drugs are those with antagonists of chemokine receptors, e.g. CCR-1, CCR-2, CCR-3, CCR-4, CCR-5, CCR-6, CCR-7, CCR-8, CCR-9 and CCR10, CXCR1, CXCR2, CXCR3, CXCR4, CXCR5, particularly CCR-5 antagonists such as Schering-Plough antagonists SC-351125, SCH—55700 and SCH-D, and Takeda antagonists such as N-[[4-[[[6,7-dihydro-2-(4-methylphenyl)-5H-benzo-cyclohepten-8-yl]carbonyl]amino]phenyl]-methyl]tetrahydro-N,N-dimethyl-2H-pyran-4-aminium chloride (TAK-770).

The structure of the active compounds identified by code numbers, generic or trade names may be taken from the actual edition of the standard compendium “The Merck Index” or from databases, e.g. Patents International (e.g. IMS World Publications).

A compound of the current invention may also be used in combination with known therapeutic processes, for example, the administration of hormones or radiation. In certain embodiments, a provided compound is used as a radiosensitizer, especially for the treatment of tumors which exhibit poor sensitivity to radiotherapy.

A compound of the current invention can be administered alone or in combination with one or more other therapeutic compounds, possible combination therapy taking the form of fixed combinations or the administration of a compound of the invention and one or more other therapeutic compounds being staggered or given independently of one another, or the combined administration of fixed combinations and one or more other therapeutic compounds. A compound of the current invention can besides or in addition be administered especially for tumor therapy in combination with chemotherapy, radiotherapy, immunotherapy, phototherapy, surgical intervention, or a combination of these. Long-term therapy is equally possible as is adjuvant therapy in the context ofothertreatment strategies, as described above. Other possible treatments are therapy to maintain the patient's status after tumor regression, or even chemopreventive therapy, for example in patients at risk.

Those additional agents may be administered separately from an inventive compound-containing composition, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

As used herein, the term “combination.” “combined,” and related terms refers to the simultaneous or sequential administration of therapeutic agents in accordance with this invention. For example, a compound of the present invention may be administered with another therapeutic agent simultaneously or sequentially in separate unit dosage forms or together in a single unit dosage form. Accordingly, the present invention provides a single unit dosage form comprising a compound of the current invention, an additional therapeutic agent, and a pharmaceutically acceptable carrier, adjuvant, or vehicle.

The amount of both an inventive compound and additional therapeutic agent (in those compositions which comprise an additional therapeutic agent as described above) that may be combined with the carrier materials to produce a single dosage form will vary depending upon the host treated and the particular mode of administration. Preferably, compositions of this invention should be formulated so that a dosage of between 0.01-100 mg/kg body weight/day of an inventive compound can be administered.

In those compositions which comprise an additional therapeutic agent, that additional therapeutic agent and the compound of this invention may act synergistically. Therefore, the amount of additional therapeutic agent in such compositions will be less than that required in a monotherapy utilizing only that therapeutic agent. In such compositions a dosage of between 0.01-1,000 μg/kg body weight/day of the additional therapeutic agent can be administered.

The amount of one or more other therapeutic agent present in the compositions of this invention may be no more than the amount that would normally be administered in a composition comprising that therapeutic agent as the only active agent. Preferably the amount of one or more other therapeutic agent in the presently disclosed compositions will range from about 50% to 100% of the amount normally present in a composition comprising that agent as the only therapeutically active agent. In some embodiments, one or more other therapeutic agent is administered at a dosage of about 50%, about 55%, about 60%, about 65%, about 70%, about 75%, about 80%, about 85%, about 90%, or about 95% of the amount normally administered for that agent. As used herein, the phrase “normally administered” means the amount an FDA approved therapeutic agent is provided for dosing per the FDA label insert.

The compounds of this invention, or pharmaceutical compositions thereof, may also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor. Implantable devices coated with a compound of this invention are another embodiment of the present invention.

### Exemplary Immuno-Oncology Agents

In some embodiments, one or more other therapeutic agent is an immuno-oncology agent. As used herein, the term “an immuno-oncology agent” refers to an agent which is effective to enhance, stimulate, and/or up-regulate immune responses in a subject. In some embodiments, the administration of an immuno-oncology agent with a compound of the invention has a synergic effect in treating a cancer.

An immuno-oncology agent can be, for example, a small molecule drug, an antibody, or a biologic or small molecule. Examples of biologic immuno-oncology agents include, but are not limited to, cancer vaccines, antibodies, and cytokines. In some embodiments, an antibody is a monoclonal antibody. In some embodiments, a monoclonal antibody is humanized or human.

In some embodiments, an immuno-oncology agent is (i) an agonist of a stimulatory (including a co-stimulatory) receptor or (ii) an antagonist of an inhibitory (including a co-inhibitory) signal on T cells, both of which result in amplifying antigen-specific T cell responses.

Certain of the stimulatory and inhibitory molecules are members of the immunoglobulin super family (IgSF). One important family of membrane-bound ligands that bind to co-stimulatory or co-inhibitory receptors is the B7 family, which includes B7-1. B7-2, B7-H1 (PD-L1), B7-DC (PD-L2), B7-H2 (ICOS-L), B7-H3, B7-H4, B7-H5 (VISTA), and B7-H6. Another family of membrane bound ligands that bind to co-stimulatory or co-inhibitory receptors is the TNF family of molecules that bind to cognate TNF receptor family members, which includes CD40 and CD40L, OX-40, OX-40L, CD70, CD27L, CD30, CD30L, 4-1BBL, CD137 (4-113B), TRAIL/Apo2-L, TRAILR1/DR4, TRAILR2/DR5, TRAILR3, TRAILR4, OPG, RANK, RANKL, TWEAKR/Fn14, TWEAK, BAFFR, EDAR, XEDAR, TAC. APRIL, BCMA, LTβR, LIGHT, DcR3, HVEM, VEGI/TLLA, TRAMP/DR3, EDAR, EDA1, XEDAR, EDA2, TNFR1, Lymphotoxin α/TNFβ, TNFR2, TNFα, LTβR, Lymphotoxin α1β2, FAS, FASL, RELT, DR6, TROY, NGFR.

In some embodiments, an immuno-oncology agent is a cytokine that inhibits T cell activation (e.g., IL-6, IL-10, TGF-β, VEGF, and other immunosuppressive cytokines) or a cytokine that stimulates T cell activation, for stimulating an immune response.

In some embodiments, a combination of a compound of the invention and an immuno-oncology agent can stimulate T cell responses. In some embodiments, an immuno-oncology agent is: (i) an antagonist of a protein that inhibits T cell activation (e.g., immune checkpoint inhibitors) such as CTLA-4, PD-1, PD-L1, PD-L2, LAG-3, TIM-3, Galectin 9, CEACAM-1, BTLA, CD69. Galectin-1, TIGIT, CD113, GPR56. VISTA, 2B4, CD48, GARP, PD1H, LAIR1, TIM-1, and TIM-4; or (ii) an agonist of a protein that stimulates T cell activation such as B7-1, B7-2, CD28, 4-1BB (CD137), 4-1BBL, ICOS, ICOS-L, OX40, OX40L, GITR, GITRL, CD70, CD27, CD40, DR3 and CD28H.

In some embodiments, an immuno-oncology agent is an antagonist of inhibitory receptors on NK cells or an agonists of activating receptors on NK cells. In some embodiments, an immuno-oncology agent is an antagonists of KIR, such as lirilumab.

In some embodiments, an immuno-oncology agent is an agent that inhibits or depletes macrophages or monocytes, including but not limited to CSF-1R antagonists such as CSF-1R antagonist antibodies including RG7155 (WO11/70024, WO11/107553, WO11/131407, WO13/87699, WO13/119716, WO13/132044) or FPA-008 (WO11/140249; WO13169264; WO14/036357).

In some embodiments, an immuno-oncology agent is selected from agonistic agents that ligate positive costimulatory receptors, blocking agents that attenuate signaling through inhibitory receptors, antagonists, and one or more agents that increase systemically the frequency of anti-tumor T cells, agents that overcome distinct immune suppressive pathways within the tumor microenvironment (e.g., block inhibitory receptor engagement (e.g., PD-L1/PD-1 interactions), deplete or inhibit Tregs (e.g., using an anti-CD25 monoclonal antibody (e.g., daclizumab) or by ex vivo anti-CD25 bead depletion), inhibit metabolic enzymes such as IDO, or reverse/prevent T cell energy or exhaustion) and agents that trigger innate immune activation and/or inflammation at tumor sites.

In some embodiments, an immuno-oncology agent is a CTLA-4 antagonist. In some embodiments, a CTLA-4 antagonist is an antagonistic CTLA-4 antibody. In some embodiments, an antagonistic CTLA-4 antibody is YERVOY (ipilimnumab) or tremelimumab.

In some embodiments, an immuno-oncology agent is a PD-1 antagonist. In some embodiments, a PD-1 antagonist is administered by infusion. In some embodiments, an immuno-oncology agent is an antibody or an antigen-binding portion thereof that binds specifically to a Programmed Death-1 (PD-1) receptor and inhibits PD-1 activity. In some embodiments, a PD-1 antagonist is an antagonistic PD-1 antibody. In some embodiments, an antagonistic PD-1 antibody is OPDIVO (nivolumab), KEYTRUDA (pembrolizumab), or MEDI-0680 (AMP-514; WO2012/145493). In some embodiments, an immuno-oncology agent may be pidilizumab (CT-011). In some embodiments, an immuno-oncology agent is a recombinant protein composed of the extracellular domain of PD-L2 (B7-DC) fused to the Fe portion of IgG1, called AMP-224.

In some embodiments, an immuno-oncology agent is a PD-L1 antagonist. In some embodiments, a PD-L1 antagonist is an antagonistic PD-L1 antibody. In some embodiments, a PD-L1 antibody is MPDL3280A (RG7446; WO2010/077634), durvalumab (MEDI4736), BMS-936559 (WO2007/005874), and MSB0010718C (WO2013/79174).

In some embodiments, an immuno-oncology agent is a LAG-3 antagonist. In some embodiments, a LAG-3 antagonist is an antagonistic LAG-3 antibody. In some embodiments, a LAG3 antibody is BMS-986016 (WO10/19570, WO14/08218), or IMP-731 or IMP-321 (WO08/132601, WO009/44273).

In some embodiments, an immuno-oncology agent is a CD137 (4-1BB) agonist. In some embodiments, a CD137 (4-1BB) agonist is an agonistic CD137 antibody. In some embodiments, a CD137 antibody is urelumab or PF-05082566 (WO12/32433).

In some embodiments, an immuno-oncology agent is a GITR agonist. In some embodiments, a GITR agonist is an agonistic GITR antibody. In some embodiments, a GITR antibody is BMS-986153, BMS-986156, TRX-518 (WO006/105021, WO009/009116), or MK-4166 (WO11/028683).

In some embodiments, an immuno-oncology agent is an indoleanine (2,3)-dioxygenase (IDO) antagonist. In some embodiments, an IDO antagonist is selected from epacadostat (INCB024360, Incyte); indoximod (NLG-8189, NewLink Genetics Corporation); capmanitib (INC280, Novartis); GDC-0919 (Genentech/Roche); PF-06840003 (Pfizer); BMS:F001287 (Bristol-Myers Squibb); Phy906/KD108 (Phytoceutica); an enzyme that breaks down kynurenine (Kynase, Kyn Therapeutics); and NLG-919 (WO09/73620, WO009/1156652, WO11/56652, WO12/142237).

In some embodiments, an immuno-oncology agent is an OX40 agonist. In some embodiments, an OX40 agonist is an agonistic OX40 antibody. In some embodiments, an OX40 antibody is MEDI-6383 or MEDI-6469.

In some embodiments, an immuno-oncology agent is an OX40L antagonist. In some embodiments, an OX40L antagonist is an antagonistic OX40 antibody. In some embodiments, an OX40L antagonist is RG-7888 (WO06/029879).

In some embodiments, an immuno-oncology agent is a CD40 agonist. In some embodiments, a CD40 agonist is an agonistic CD40 antibody. In some embodiments, an immuno-oncology agent is a CD40 antagonist. In some embodiments, a CD40 antagonist is an antagonistic CD40 antibody. In some embodiments, a CD40 antibody is lucatumumab or dacetuzumab.

In some embodiments, an immuno-oncology agent is a CD27 agonist. In some embodiments, a CD27 agonist is an agonistic CD27 antibody. In some embodiments, a CD27 antibody is varlilumab.

In some embodiments, an immuno-oncology agent is MGA271 (to B7H3) (WO11/109400).

In some embodiments, an immuno-oncology agent is abagovomab, adecatumumab, afutuzumab, alemtuzumab, anatumomab mafenatox, apolizumab, atezolimab, avelumab, blinatumomab, BMS-936559, catumaxomab, durvalumab, epacadostat, epratuzumab, indoximod, inotuzumab ozogamicin, intelumumab, ipilimumab, isatuximab, lambrolizumab, MED14736, MPDL3280A, nivolumab, obinutuzumab, ocaratuzumab, ofatumumab, olatatumab, pembrolizumab, pidilizumab, rituximab, ticilimumab, samalizumab, or tremelimumab.

In some embodiments, an immuno-oncology agent is an immunostimulatory agent. For example, antibodies blocking the PD-1 and PD-L1 inhibitory axis can unleash activated tumor-reactive T cells and have been shown in clinical trials to induce durable anti-tumor responses in increasing numbers of tumor histologies, including some tumor types that conventionally have not been considered immunotherapy sensitive. See, e.g., Okazaki, T. et al. (2013) Nat. Immunol. 14, 1212-1218; Zou et al. (2016) Sci. Transl. Med. 8. The anti-PD-1 antibody nivolumab (Opdivo®, Bristol-Myers Squibb, also known as ONO-4538, MDX1106 and BMS-936558), has shown potential to improve the overall survival in patients with RCC who had experienced disease progression during or after prior anti-angiogenic therapy.

In some embodiments, the immunomodulatory therapeutic specifically induces apoptosis of tumor cells. Approved immunomodulatory therapeutics which may be used in the present invention include pomalidomide (Pomalyst®, Celgene); lenalidomide (Revlimid®. Celgene); ingenol mebutate (Picato®, LEO Phanna).

In some embodiments, an immuno-oncology agent is a cancer vaccine. In some embodiments, the cancer vaccine is selected from sipuleucel-T (Provenge®, Dendreon/Valeant Pharmaceuticals), which has been approved for treatment of asymptomatic, or minimally symptomatic metastatic castrate-resistant (hormone-refractory) prostate cancer; and talimogene laherparepvec (Imlygic®, BioVex/Amgen, previously known as T-VEC), a genetically modified oncolytic viral therapy approved for treatment of unresectable cutaneous, subcutaneous and nodal lesions in melanoma. In some embodiments, an immuno-oncology agent is selected from an oncolytic viral therapy such as pexastimogene devacirepvec (PexaVec/JX-594, SillaJen/formerly Jennerex Biotherapeutics), a thymidine kinase- (TK-) deficient vaccinia virus engineered to express GM-CSF, for hepatocellular carcinoma (NCT02562755) and melanoma (NCT00429312); pelarcorep (Rcolysin®, Oncolytics Biotech), a variant of respiratory enteric orphan virus (reovirus) which does not replicate in cells that are not RAS-activated, in numerous cancers, including colorectal cancer (NCT01622543); prostate cancer (NCT01619813); head and neck squamous cell cancer (NCT01166542); pancreatic adenocarcinoma (NCT00998322); and non-small cell lung cancer (NSCLC) (NCT 00861627); enadenotucirev (NG-348, PsiOxus, formerly known as ColoAd1), an adenovirus engineered to express a full length CD80 and an antibody fragment specific for the T-cell receptor CD3 protein, in ovarian cancer (NCT02028117); metastatic or advanced epithelial tumors such as in colorectal cancer, bladder cancer, head and neck squamous cell carcinoma and salivary gland cancer (NCT02636036); ONCOS-102 (Targovax/formerly Oncos), an adenovirus engineered to express GM-CSF, in melanoma (NCT03003676); and peritoneal disease, colorectal cancer or ovarian cancer (NCT02963831); GL-ONC1 (GLV-1h68/GLV-1h153, Genelux GmbH), vaccinia viruses engineered to express beta-galactosidase (beta-gal)/beta-glucoronidase or beta-gal/human sodium iodide symporter (hNIS), respectively, were studied in peritoneal carcinomatosis (NCT01443260); fallopian tube cancer, ovarian cancer (NCT 02759588); or CG0070 (Cold Genesys), an adenovirus engineered to express GM-CSF, in bladder cancer (NCT02365818).

In some embodiments, an immuno-oncology agent is selected from JX-929 (SillaJen/formerly Jennerex Biotherapeutics), a TK- and vaccinia growth factor-deficient vaccinia virus engineered to express cytosine deaminase, which is able to convert the prodrug 5-fluorocytosine to the cytotoxic drug 5-fluorouracil; TG01 and TG02 (Targovax/formerly Oncos), peptide-based immunotherapy agents targeted for difficult-to-treat RAS mutations; and TILT-123 (TILT Biotherapeutics), an engineered adenovirus designated: Ad5/3-E2F-delta24-hTNFα-IRES-hIL20; and VSV-GP (ViraTherapeutics) a vesicular stomatitis virus (VSV) engineered to express the glycoprotein (GP) of lymphocytic choriomeningitis virus (LCMV), which can be further engineered to express antigens designed to raise an antigen-specific CD8⁺ T cell response.

In some embodiments, an immuno-oncology agent is a T-cell engineered to express a chimeric antigen receptor, or CAR. The T-cells engineered to express such chimeric antigen receptor are referred to as a CAR-T cells.

CARs have been constructed that consist of binding domains, which may be derived from natural ligands, single chain variable fragments (scFv) derived from monoclonal antibodies specific for cell-surface antigens, fused to endodomains that are the functional end of the T-cell receptor (TCR), such as the CD3-zeta signaling domain from TCRs, which is capable of generating an activation signal in T lymphocytes. Upon antigen binding, such CARs link to endogenous signaling pathways in the effector cell and generate activating signals similar to those initiated by the TCR complex.

For example, in some embodiments the CAR-T cell is one of those described in U.S. Pat. No. 8,906,682 (June; hereby incorporated by reference in its entirety), which discloses CAR-T cells engineered to comprise an extracellular domain having an antigen binding domain (such as a domain that binds to CD19), fused to an intracellular signaling domain of the T cell antigen receptor complex zeta chain (such as CD3 zeta). When expressed in the T cell, the CAR is able to redirect antigen recognition based on the antigen binding specificity. In the case of CD19, the antigen is expressed on malignant B cells. Over 200 clinical trials are currently in progress employing CAR-T in a wide range of indications. [https://clinicaltrials.gov/ct2/results?term=chimeric+antigen+receptors&pg=1].

In some embodiments, an immunostimulatory agent is an activator of retinoic acid receptor-related orphan receptor γ (RORγt). RORγt is a transcription factor with key roles in the differentiation and maintenance of Type 17 effector subsets of CD4+(Th17) and CD8+(Tc17) T cells, as well as the differentiation of IL-17 expressing innate immune cell subpopulations such as NK cells. In some embodiments, an activator of RORγt is LYC-55716 (Lycera), which is currently being evaluated in clinical trials for the treatment of solid tumors (NCT02929862).

In some embodiments, an immunostimulatory agent is an agonist or activator of a toll-like receptor (TLR). Suitable activators of TLRs include an agonist or activator of TLR9 such as SD-101 (Dynavax). SD-101 is an immunostimulatory CpG which is being studied for B-cell, follicular and other lymphomas (NCT02254772). Agonists or activators of TLR8 which may be used in the present invention include motolimod (VTX-2337, VentiRx Pharmaceuticals) which is being studied for squamous cell cancer of the head and neck (NCT02124850) and ovarian cancer (NCT02431559).

Other immuno-oncology agents that may be used in the present invention include urelumab (BMS-663513, Bristol-Myers Squibb), an anti-CD137 monoclonal antibody; varlilumab (CDX-1127, Celldex Therapeutics), an anti-CD27 monoclonal antibody; BMS-986178 (Bristol-Myers Squibb), an anti-OX40 monoclonal antibody; lirilumab (IPH2102/BMS-986015, Innate Pharma, Bristol-Myers Squibb), an anti-KIR monoclonal antibody; monalizumab (IPH2201, Innate Pharma, AstraZeneca) an anti-NKG2A monoclonal antibody; andecaliximab (GS-5745, Gilead Sciences), an anti-MMP9 antibody; MK-4166 (Merck & Co.), an anti-GITR monoclonal antibody.

In some embodiments, an immunostimulatory agent is selected from elotuzumab, mifamurtide, an agonist or activator of a toll-like receptor, and an activator of RORγt.

In some embodiments, an immunostimulatory therapeutic is recombinant human interleukin 15 (rhIL-15). rhIL-15 has been tested in the clinic as a therapy for melanoma and renal cell carcinoma (NCT01021059 and NCT01369888) and leukemias (NCT02689453). In some embodiments, an immunostimulatory agent is recombinant human interleukin 12 (rhIL-12). In some embodiments, an IL-15 based immunotherapeutic is heterodimeric IL-15 (hetIL-15, Novartis/Admune), a fusion complex composed of a synthetic form of endogenous IL-15 complexed to the soluble IL-15 binding protein IL-15 receptor alpha chain (IL15:sIL-15R^{A}), which has been tested in Phase 1 clinical trials for melanoma, renal cell carcinoma, non-small cell lung cancer and head and neck squamous cell carcinoma (NCT02452268). In some embodiments, a recombinant human interleukin 12 (rhIL-12) is NM-IL-12 (Neumedicines, Inc.), NCT02544724, or NCT02542124.

In some embodiments, an immuno-oncology agent is selected from those described in Jerry L. Adams et al., “Big opportunities for small molecules in immuno-oncology,” Cancer Therapy 2015, Vol. 14, pages 603-622, the content of which is incorporated herein by reference in its entirety. In some embodiments, an immuno-oncology agent is selected from the examples described in Table 1 of Jerry L. Adams et al. In some embodiments, an immuno-oncology agent is a small molecule targeting an immuno-oncology target selected from those listed in Table 2 of Jerry L. Adams ET. AL. In some embodiments, an immuno-oncology agent is a small molecule agent selected from those listed in Table 2 of Jerry L. Adams et al.

In some embodiments, an immuno-oncology agent is selected from the small molecule immuno-oncology agents described in Peter L. Toogood, “Small molecule immuno-oncology therapeutic agents,” Bioorganic & Medicinal Chemistry Letters 2018. Vol. 28, pages 319-329, the content of which is incorporated herein by reference in its entirety. In some embodiments, an immuno-oncology agent is an agent targeting the pathways as described in Peter L. Toogood.

In some embodiments, an immuno-oncology agent is selected from those described in Sandra L. Ross et al., “Bispecific T cell engager (BiTE®) antibody constructs can mediate bystander tumor cell killing”. PLoS ONE 12(8): e0183390, the contents of which is incorporated herein by reference in its entirety. In some embodiments, an immuno-oncology agent is a bispecific T cell engager (BiTE®) antibody construct. In some embodiments, a bispecific T cell engager (BiTE®) antibody construct is a CD19/CD3 bispecific antibody construct. In some embodiments, a bispecific T cell engager (BiTE®) antibody construct is an EGFR/CD3 bispecific antibody construct. In some embodiments, a bispecific T cell engager (BiTE®) antibody construct activates T cells. In some embodiments, a bispecific T cell engager (BiTE®) antibody construct activates T cells, which release cytokines inducing upregulation of intercellular adhesion molecule 1 (ICAM-1) and FAS on bystander cells. In some embodiments, a bispecific T cell engager (BiTE®) antibody construct activates T cells which result in induced bystander cell lysis. In some embodiments, the bystander cells are in solid tumors. In some embodiments, the bystander cells being lysed are in proximity to the BiTE®-activated T cells. In some embodiment, the bystander cells comprises tumor-associated antigen (TAA) negative cancer cells. In some embodiment, the bystander cells comprise EGFR-negative cancer cells. In some embodiments, an immuno-oncology agent is an antibody which blocks the PD-L1/PD1 axis and/or CTLA4. In some embodiments, an immuno-oncology agent is an ex-vivo expanded tumor-infiltrating T cell. In some embodiments, an immuno-oncology agent is a bispecific antibody construct or chimeric antigen receptors (CARs) that directly connect T cells with tumor-associated surface antigens (TAAs).

### Exemplary Immune Checkpoint Inhibitors

In some embodiments, an immuno-oncology agent is an immune checkpoint inhibitor as described herein.

The term “checkpoint inhibitor” as used herein relates to agents useful in preventing cancer cells from avoiding the immune system of the patient. One of the major mechanisms of anti-tumor immunity subversion is known as “T-cell exhaustion,” which results from chronic exposure to antigens that has led to up-regulation of inhibitory receptors. These inhibitory receptors serve as immune checkpoints in order to prevent uncontrolled immune reactions.

PD-1 and co-inhibitory receptors such as cytotoxic T-lymphocyte antigen 4 (CTLA-4, B and T Lymphocyte Attenuator (BTLA; CD272), T cell Immunoglobulin and Mucin domain-3 (Tim-3), Lymphocyte Activation Gene-3 (Lag-3; CD223), and others are often referred to as a checkpoint regulators. They act as molecular “gatekeepers” that allow extracellular information to dictate whether cell cycle progression and other intracellular signaling processes should proceed.

In some embodiments, an immune checkpoint inhibitor is an antibody to PD-1. PD-1 binds to the programmed cell death 1 receptor (PD-1) to prevent the receptor from binding to the inhibitory ligand PDL-1, thus overriding the ability of tumors to suppress the host anti-tumor immune response.

In one aspect, the checkpoint inhibitor is a biologic therapeutic or a small molecule. In another aspect, the checkpoint inhibitor is a monoclonal antibody, a humanized antibody, a fully human antibody, a fusion protein or a combination thereof. In a further aspect, the checkpoint inhibitor inhibits a checkpoint protein selected from CTLA-4, PDL1, PDL2, PD1, B7-H3, B7-H4, BTLA, HVEM, TIM3, GAL9, LAG3, VISTA, KIR, 2B4. CD160, CGEN-15049, CHK 1, CHK2, A2aR, B-7 family ligands or a combination thereof. In an additional aspect, the checkpoint inhibitor interacts with a ligand of a checkpoint protein selected from CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In an aspect, the checkpoint inhibitor is an immunostimulatory agent, a T cell growth factor, an interleukin, an antibody, a vaccine or a combination thereof. In a further aspect, the interleukin is IL-7 or IL-15. In a specific aspect, the interleukin is glycosylated IL-7. In an additional aspect, the vaccine is a dendritic cell (DC) vaccine.

Checkpoint inhibitors include any agent that blocks or inhibits in a statistically significant manner, the inhibitory pathways of the immune system. Such inhibitors may include small molecule inhibitors or may include antibodies, or antigen binding fragments thereof, that bind to and block or inhibit immune checkpoint receptors or antibodies that bind to and block or inhibit immune checkpoint receptor ligands. Illustrative checkpoint molecules that may be targeted for blocking or inhibition include, but are not limited to, CTLA-4, PDL1, PDL2, PD1, B7-H3, B7-H4, BTLA, HVEM, GAL9, LAG3, TIM3, VISTA, KIR, 2B4 (belongs to the CD2 family of molecules and is expressed on all NK, γδ, and memory CD8⁺ (αβ) T cells), CD160 (also referred to as BY55), CGEN-15049, CHK 1 and CHK2 kinases, A2aR, and various B-7 family ligands. B7 family ligands include, but are not limited to, B7-1, B7-2. B7-DC, B7-H1, B7-H2, B7-H3, B7-H4, B7-H5, B7-H6 and B7-H7. Checkpoint inhibitors include antibodies, or antigen binding fragments thereof, other binding proteins, biologic therapeutics, or small molecules, that bind to and block or inhibit the activity of one or more of CTLA-4, PDL1, PDL2, PD1, BTLA, HVEM, TIM3, GAL9, LAG3, VISTA, KIR, 2B4, CD 160 and CGEN-15049. Illustrative immune checkpoint inhibitors include Tremelimumab (CTLA-4 blocking antibody), anti-OX40, PD-L1 monoclonal Antibody (Anti-B7-H1; MED14736), MK-3475 (PD-1 blocker), Nivolumab (anti-PD1 antibody), CT-011 (anti-PD1 antibody). BY55 monoclonal antibody, AMP224 (anti-PDL1 antibody), BMS- 936559 (anti-PDL1 antibody), MPLDL3280A (anti-PDL1 antibody), MSB0010718C (anti-PDL1 antibody), and ipilimumab (anti-CTLA-4 checkpoint inhibitor). Checkpoint protein ligands include, but are not limited to PD-L1, PD-L2, B7-H3, B7-H4, CD28, CD86 and TIM-3.

In certain embodiments, the immune checkpoint inhibitor is selected from a PD-1 antagonist, a PD-L1 antagonist, and a CTLA-4 antagonist. In some embodiments, the checkpoint inhibitor is selected from the group consisting of nivolumab (Opdivo®), ipilimumab (Yervoy®), and pembrolizumab (Keytruda®). In some embodiments, the checkpoint inhibitor is selected from nivolumab (anti-PD-1 antibody, Opdivo®, Bristol-Myers Squibb); pembrolizumab (anti-PD-1 antibody, Keytruda®, Merck); ipilimumab (anti-CTLA-4 antibody, Yervoy®, Bristol-Myers Squibb); durvalumab (anti-PD-L1 antibody, Imfinzi®, AstraZeneca); and atezolizumab (anti-PD-L1 antibody, Tecentriq®, Genentech).

In some embodiments, the checkpoint inhibitor is selected from the group consisting of lambrolizumab (MK-3475), nivolumab (BMS-936558), pidilizumab (CT-011), AMP-224, MDX-1105, MED14736, MPDL3280A, BMS-936559, ipilimumab, lirlumab, IPH2101, pembrolizumab (Keytruda®), and tremelimumab.

In some embodiments, an immune checkpoint inhibitor is REGN2810 (Regeneron), an anti-PD-1 antibody tested in patients with basal cell carcinoma (NCT03132636); NSCLC (NCT03088540); cutaneous squamous cell carcinoma (NCT02760498); lymphoma (NCT02651662); and melanoma (NCT03002376); pidilizumab (CureTech), also known as CT-011, an antibody that binds to PD-1, in clinical trials for diffuse large B-cell lymphoma and multiple myeloma; avelumab (Bavencio®). Pfizer/Merck KGaA), also known as MSB0010718C), a fully human IgG1 anti-PD-L1 antibody, in clinical trials for non-small cell lung cancer, Merkel cell carcinoma, mesothelioma, solid tumors, renal cancer, ovarian cancer, bladder cancer, head and neck cancer, and gastric cancer; or PDR001 (Novartis), an inhibitory antibody that binds to PD-1, in clinical trials for non-small cell lung cancer, melanoma, triple negative breast cancer and advanced or metastatic solid tumors. Tremelimumab (CP-675,206; Astrazeneca) is a fully human monoclonal antibody against CTLA-4 that has been in studied in clinical trials for a number of indications, including: mesothelioma, colorectal cancer, kidney cancer, breast cancer, lung cancer and non-small cell lung cancer, pancreatic ductal adenocarcinoma, pancreatic cancer, germ cell cancer, squamous cell cancer of the head and neck, hepatocellular carcinoma, prostate cancer, endometrial cancer, metastatic cancer in the liver, liver cancer, large B-cell lymphoma, ovarian cancer, cervical cancer, metastatic anaplastic thyroid cancer, urothelial cancer, fallopian tube cancer, multiple myeloma, bladder cancer, soft tissue sarcoma, and melanoma. AGEN-1884 (Agenus) is an anti-CTLA4 antibody that is being studied in Phase 1 clinical trials for advanced solid tumors (NCT02694822).

In some embodiments, a checkpoint inhibitor is an inhibitor of T-cell immunoglobulin mucin containing protein-3 (TIM-3). TIM-3 inhibitors that may be used in the present invention include TSR-022, LY3321367 and MBG453. TSR-022 (Tesaro) is an anti-TIM-3 antibody which is being studied in solid tumors (NCT02817633). LY3321367 (Eli Lilly) is an anti-TIM-3 antibody which is being studied in solid tumors (NCT03099109). MBG453 (Novartis) is an anti-TIM-3 antibody which is being studied in advanced malignancies (NCT02608268).

In some embodiments, a checkpoint inhibitor is an inhibitor of T cell immunoreceptor with Ig and ITIM domains, or TIGIT, an immune receptor on certain T cells and NK cells. TIGIT inhibitors that may be used in the present invention include BMS-986207 (Bristol-Myers Squibb), an anti-TIGIT monoclonal antibody (NCT02913313); OMP-313M32 (Oncomed); and anti-TIGIT monoclonal antibody (NCT03119428).

In some embodiments, a checkpoint inhibitor is an inhibitor of Lymphocyte Activation Gene-3 (LAG-3). LAG-3 inhibitors that may be used in the present invention include BMS-986016 and REGN3767 and IMP321. BMS-986016 (Bristol-Myers Squibb), an anti-LAG-3 antibody, is being studied in glioblastoma and gliosarcoma (NCT02658981). REGN3767 (Regeneron), is also an anti-LAG-3 antibody, and is being studied in malignancies (NCT03005782). IMP321 (Immutep S. A.) is an LAG-3-Ig fusion protein, being studied in melanoma (NCT02676869); adenocarcinoma (NCT02614833); and metastatic breast cancer (NCT00349934).

Checkpoint inhibitors that may be used in the present invention include OX40 agonists. OX40 agonists that are being studied in clinical trials include PF-04518600/PF-8600 (Pfizer), an agonistic anti-OX40 antibody, in metastatic kidney cancer (NCT03092856) and advanced cancers and neoplasms (NCT02554812; NCT05082566); GSK3174998 (Merck), an agonistic anti-OX40 antibody, in Phase 1 cancer trials (NCT02528357); MED10562 (Medimmune/AstraZeneca), an agonistic anti-OX40 antibody, in advanced solid tumors (NCT02318394 and NCT02705482); MEDI6469, an agonistic anti-OX40 antibody (Medimmune/AstraZeneca), in patients with colorectal cancer (NCT02559024), breast cancer (NCT01862900), head and neck cancer (NCT02274155) and metastatic prostate cancer (NCT01303705); and BMS-986178 (Bristol-Myers Squibb) an agonistic anti-OX40 antibody, in advanced cancers (NCT02737475).

Checkpoint inhibitors that may be used in the present invention include CD137 (also called 4-1BB) agonists. CD137 agonists that are being studied in clinical trials include utomilumab (PF-05082566. Pfizer) an agonistic anti-CD137 antibody, in diffuse large B-cell lymphoma (NCT02951156) and in advanced cancers and neoplasms (NCT02554812 and NCT05082566); urelumab (BMS-663513, Bristol-Myers Squibb), an agonistic anti-CD137 antibody, in melanoma and skin cancer (NCT02652455) and glioblastoma and gliosarcoma (NCT02658981).

Checkpoint inhibitors that may be used in the present invention include CD27 agonists. CD27 agonists that are being studied in clinical trials include varlilumab (CDX-1127. Celldex Therapeutics) an agonistic anti-CD27 antibody, in squamous cell head and neck cancer, ovarian carcinoma, colorectal cancer, renal cell cancer, and glioblastoma (NCT02335918); lymphomas (NCT01460134); and glioma and astrocytoma (NCT02924038).

Checkpoint inhibitors that may be used in the present invention include glucocorticoid-induced tumor necrosis factor receptor (GITR) agonists. GITR agonists that are being studied in clinical trials include TRX518 (Leap Therapeutics), an agonistic anti-GITR antibody, in malignant melanoma and other malignant solid tumors (NCT01239134 and NCT02628574); GWN323 (Novartis), an agonistic anti-GITR antibody, in solid tumors and lymphoma (NCT 02740270); INCAGN01876 (Incyte/Agenus), an agonistic anti-GITR antibody, in advanced cancers (NCT02697591 and NCT03126110); MK-4166 (Merck), an agonistic anti-GITR antibody, in solid tumors (NCT02132754) and MEDI1873 (Medimmune/AstraZeneca), an agonistic hexameric GITR-ligand molecule with a human IgG1 Fc domain, in advanced solid tumors (NCT02583165).

Checkpoint inhibitors that may be used in the present invention include inducible T-cell co-stimulator (ICOS, also known as CD278) agonists. ICOS agonists that are being studied in clinical trials include MEDI-570 (Medimmune), an agonistic anti-ICOS antibody, in lymphomas (NCT02520791); GSK3359609 (Merck), an agonistic anti-ICOS antibody, in Phase 1 (NCT02723955); JTX-2011 (Jounce Therapeutics), an agonistic anti-ICOS antibody, in Phase 1 (NCT02904226).

Checkpoint inhibitors that may be used in the present invention include killer IgG-like receptor (KIR) inhibitors. KIR inhibitors that are being studied in clinical trials include lirilumab (IPH2102/BMS-986015, Innate Pharma/Bristol-Myers Squibb), an anti-KIR antibody, in leukemias (NCT01687387. NCT02399917, NCT02481297, NCT02599649), multiple myeloma (NCT02252263), and lymphoma (NCT01592370); IPH2101 (1-7F9, Innate Pharma) in myeloma (NCT01222286 and NCT01217203); and IPH4102 (Innate Pharma), an anti-KIR antibody that binds to three domains of the long cytoplasmic tail (KIR3DL2), in lymphoma (NCT02593045).

Checkpoint inhibitors that may be used in the present invention include CD47 inhibitors of interaction between CD47 and signal regulatory protein alpha (SIRPa). CD47/SIRPa inhibitors that are being studied in clinical trials include ALX-148 (Alexo Therapeutics), an antagonistic variant of (SIRPa) that binds to CD47 and prevents CD47/SIRPa-mediated signaling, in phase 1 (NCT03013218); TTI-621 (SIRPa-Fc, Trillium Therapeutics), a soluble recombinant fusion protein created by linking the N-terminal CD47-binding domain of SIRPa with the Fc domain of human IgGI, acts by binding human CD47, and preventing it from delivering its “do not eat” signal to macrophages, is in clinical trials in Phase 1 (NCT02890368 and NCT02663518); CC-90002 (Celgene), an anti-CD47 antibody, in leukemias (NCT02641002); and Hu5F9-G4 (Forty Seven, Inc.), in colorectal neoplasms and solid tumors (NCT02953782), acute myeloid leukemia (NCT02678338) and lymphoma (NCT02953509).

Checkpoint inhibitors that may be used in the present invention include CD73 inhibitors. CD73 inhibitors that are being studied in clinical trials include MED19447 (Medimmune), an anti-CD73 antibody, in solid tumors (NCT02503774); and BMS-986179 (Bristol-Myers Squibb), an anti-CD73 antibody, in solid tumors (NCT02754141).

Checkpoint inhibitors that may be used in the present invention include agonists of stimulator of interferon genes protein (STING, also known as transmembrane protein 173, or TMEM173). Agonists of STING that are being studied in clinical trials include MK-1454 (Merck), an agonistic synthetic cyclic dinucleotide, in lymphoma (NCT03010176); and ADU-S100 (MIW815, Aduro Biotech/Novartis), an agonistic synthetic cyclic dinucleotide, in Phase 1 (NCT02675439 and NCT03172936).

In some embodiments, STAT6 inhibition/degradation can significantly enhance CDN-induced STING signaling and antitumor immunity (Pei et al., Can. Lett. 2019, 450:110).

Checkpoint inhibitors that may be used in the present invention include CSF1R inhibitors. CSF1R inhibitors that are being studied in clinical trials include pexidartinib (PLX3397, Plexxikon), a CSF1R small molecule inhibitor, in colorectal cancer, pancreatic cancer, metastatic and advanced cancers (NCT02777710) and melanoma, non-small cell lung cancer, squamous cell head and neck cancer, gastrointestinal stromal tumor (GIST) and ovarian cancer (NCT02452424); and IMC-CS4 (LY3022855, Lilly), an anti-CSF-1R antibody, in pancreatic cancer (NCT03153410), melanoma (NCT03101254), and solid tumors (NCT02718911); and BLZ945 (4-[2((1R,2R)-2-hydroxycyclohexylamino)-benzothiazol-6-yloxyl]-pyridine-2-carboxylic acid methylamide, Novartis), an orally available inhibitor of CSF1R, in advanced solid tumors (NCT02829723).

Checkpoint inhibitors that may be used in the present invention include NKG2A receptor inhibitors. NKG2A receptor inhibitors that are being studied in clinical trials include monalizumab (IPH2201. Innate Pharma), an anti-NKG2A antibody, in head and neck neoplasms (NCT02643550) and chronic lymphocytic leukemia (NCT02557516).

In some embodiments, the immune checkpoint inhibitor is selected from nivolumab, pembrolizumab, ipilimumab, avelumab, durvalumab, atezolizumab, or pidilizumab.

### EXEMPLIFICATION

### General Synthetic Methods

The following examples are intended to illustrate the invention and are not to be construed as being limitations thereon. Temperatures are given in degrees centigrade. If not mentioned otherwise, all evaporations are performed under reduced pressure, preferably between about 15 mm Hg and 100 mm Hg (=20-133 mbar). The structure of final products, intermediates and starting materials is confirmed by standard analytical methods, e.g., microanalysis and spectroscopic characteristics, e.g., MS, IR, NMR. Abbreviations used are those conventional in the art.

All starting materials, building blocks, reagents, acids, bases, dehydrating agents, solvents, and catalysts utilized to synthesis the compounds of the present invention are either commercially available or can be produced by organic synthesis methods known to one of ordinary skill in the art (Houben-Weyl 4th Ed. 1952, Methods of Organic Synthesis, Thieme, Volume 21). Further, the compounds of the present invention can be produced by organic synthesis methods known to one of ordinary skill in the art as shown in the following examples.

All reactions are carried out under nitrogen or argon unless otherwise stated.

Proton NMR (¹H NMR) is conducted in deuterated solvent. In certain compounds disclosed herein, one or more ¹H shifts overlap with residual proteo solvent signals; these signals have not been reported in the experimental provided hereinafter.

TABLE 2
Analytical instruments

| | |
|---|---|
| LCMS | Shimadzu UFLC MS: LCMS-2020 |
| | Agilent Technologies 1200 series MS: Agilent |
| | Technologies 6110 |
| | Agilent Technologies 1200 series MS: LC/MSD VL |
| NMR | BRUKER AVANCE III/400; Frequency (MHz) 400.13; |
| | Nucleus: 1H; Number of Transients: 8 |
| Prep-HPLC | Gilson GX-281 systems: instruments GX-A, GX-B, GX-C, |
| | GX-D, GX-E, GX-F, GX-G and GX-H |
| GCMS | SHIMADZU GCMS-QP2010 Ultra |
| Analytical | Agilent Technologies 1290 Infinity |
| cSFC | |
| Prep-cSFC | Waters SFC Prep 80 |

For acidic LCMS data: LCMS was recorded on an Agilent 1200 Series LC/MSD or Shimadzu LCMS 2020 equipped with electro-spray ionization and quadruple MS detector [ES+ve to give MH+] and equipped with Chromolith Flash RP-18e 25*2.0 mm, eluting with 0.0375 vol % TEA in water (solvent A) and 0.01875 vol % TFA in acetonitrile (solvent B). Other LCMS was recorded on an Agilent 1290 Infinity RRLC attached with Agilent 6120 Mass detector. The column used was BEH C18 50*2.1 mm, 1.7 micron. Column flow was 0.55 ml/min and mobile phase were used (A) 2 mM Ammonium Acetate in 0.1% Formic Acid in Water and (B) 0.1% Formic Acid in Acetonitrile.

For basic LCMS data: LCMS was recorded on an Agilent 1200 Series LC/MSD or Shimadzu LCMS 2020 equipped with electro-spray ionization and quadruple MS detector [ES+ve to give MH+] and equipped with Xbridge C18, 2.1×50 mm columns packed with 5 mm C18-coated silica or Kinetex EVO C18 2.1×30 mm columns packed with 5 mm C18-coated silica, eluting with 0.05 vol % NH3·H₂O in water (solvent A) and acetonitrile (solvent B).

HPLC Analytical Method: HPLC was carried out on X Bridge C18 150*4.6 mm, 5 micron. Column flow was 1.0 ml/min and mobile phase were used (A) 0.1% Ammonia in water and (B) 0.1% Ammonia in Acetonitrile.

Prep HPLC Analytical Method: The compound was purified on Shimadzu LC-20AP and UV detector. The column used was X-BRIDGE C18 (250*19)mm, 5. Column flow was 16.0 ml/min. Mobile phase were used (A) 0.1% Formic Acid in Water and (B) Acetonitrile Basic method used (A) 5 mM ammonium bicarbonate and 0.1% NH3 in Water and (B) Acetonitrile or (A) 0.1% Ammonium Hydroxide in Water and (B) Acetonitrile. The UV spectra were recorded at 202 nm & 254 nm.

NMR Method: The 1H NMR spectra were recorded on a Bruker Ultra Shield Advance 400 MHz/5 mm Probe (BBFO). The chemical shifts are reported in part-per-million.

In some instances, intermediates and compounds described in the examples comprise one or more stereocenters and more than one enantiomer/diastereomer was produced. In some embodiments, these enantiomers/diastereomers were separated and isolated, although stereochemistry was not resolved. Unless otherwise stated, stereochemistry was assigned arbitrarily. For intermediates, each enantiomer/diastereomer with arbitrarily assigned stereochemistry may result in a final compound (e.g., assigned a “I—” number), which also maintains the arbitrarily assigned stereochemistry. Accordingly, any compound with arbitrarily assigned stereochemistry or produced from an intermediate with arbitrarily assigned stereochemistry may be depicted herein as a certain stereoisomer, but it is understood that such compound may be the other stereoisomer (i.e., enantiomer or diastereomer).

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein.

### Intermediates

### 2-(3-chloro-4-(2,6-dioxopiperidin-3-yl)phenyl)acetaldehyde (Intermediate A)

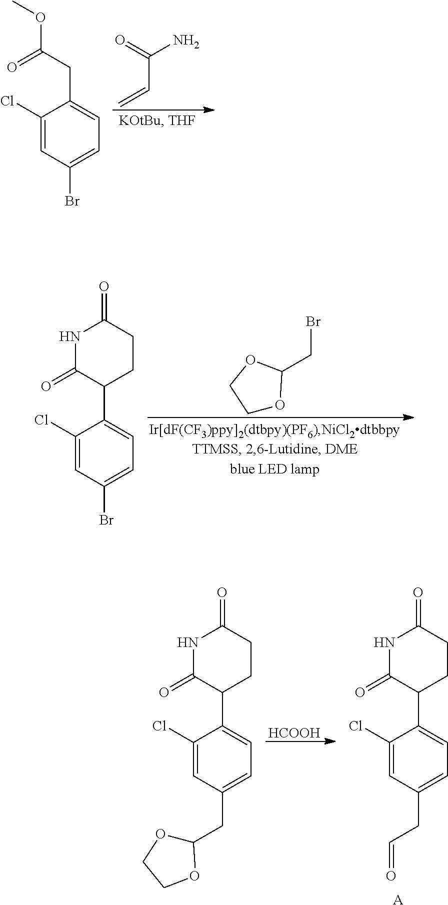

### Step 1—3-(4-bromo-2-chlorophenyl)piperidine-2,6-dione

A mixture of methyl 2-(4-bromo-2-chloro-phenyl)acetate (35 g, 130 mmol, CAS #849934-94-7) and prop-2-enamide (9.44 g, 133 mmol, 9.17 mL) in THF (350 mL) was degassed and purged with N₂three times. Then t-BuOK (16.4 g, 146 mmol) was added at 0° C., and the mixture was stirred at 50° C. for 3 h under N₂ atmosphere. On completion, the mixture was diluted with NH₄Cl solution (100 mL) and extracted with ethyl acetate (100 mL×2), dried over sodium sulfate, filtered, and concentrated under reduced pressure to give a crude product. The crude product was triturated with EtOAc:MTBE=1:2 to afford the title compound (29.2 g, 65% yield) as a white solid. LC-MS (ESI⁻) m/z 302.1 (M+H)⁺.

### Step 2—3-[2-chloro-4-(1,3-dioxolan-2-ylmethyl)phenyl]piperidine-2,6-dione

To an 50 mL vial equipped with a stir bar was added 2-(2-bromoethyl)-1,3-dioxolane (1.63 g, 9.02 mmol, 1.08 mL, CAS #4360-63-8), 3-(4-bromo-2-chloro-phenyl)piperidine-2,6-dione (2.1 g, 6.94 mmol), Ir[dF(CF₃)ppy]₂(dtbpy)(PF₆) (77.8 mg, 69.4 μmol), NiCl₂·dtbbpy (41.4 mg, 104 μmol), TTMSS (1.73 g, 6.94 mmol, 2.14 mL), and 2,6-Lutidine (1.49 g, 13.8 mmol, 1.62 mL) in DME (20 mL). The vial was sealed and placed under nitrogen. The reaction was stirred and irradiated with a blue 10 W LED lamp (3 cm away), with cooling water to keep the reaction temperature at 25° C. for 14 h. On completion, the mixture was diluted with NH₄Cl solution (100 mL) and extracted with ethyl acetate (100 mL×2), dried over sodium sulfate, filtered, and concentrated under reduced pressure to give a crude residue. The residue was purified by silica gel column chromatography (petroleum ether/ethyl acetate=3/1 to 0/1) to afford the title compound (1.3 g, 56% yield) as a white solid. LC-MS (ESI+) m/z 310.0 (M+H)⁺.

### Step 3—2-(3-Chloro-4-(2,6-dioxopiperidin-3-yl)phenyl)acetaldehyde

To a solution of 3-[2-chloro-4-(1,3-dioxolan-2-ylmethyl)phenyl]piperidine-2,6-dione (400 mg, 1.29 mmol) was added FA (4 mL). The mixture was then stirred at 40° C. for 2 h. On completion, the reaction mixture was filtered, and the filtrate was concentrated to give the title compound (440 mg, FA) as a white solid. LC-MS (ESI⁺) m/z 266.0 (M+H)⁺.

### 2-(4-(2,6-dioxopiperidin-3-yl)-2-fluorophenyl)acetaldehyde (Intermediate B)

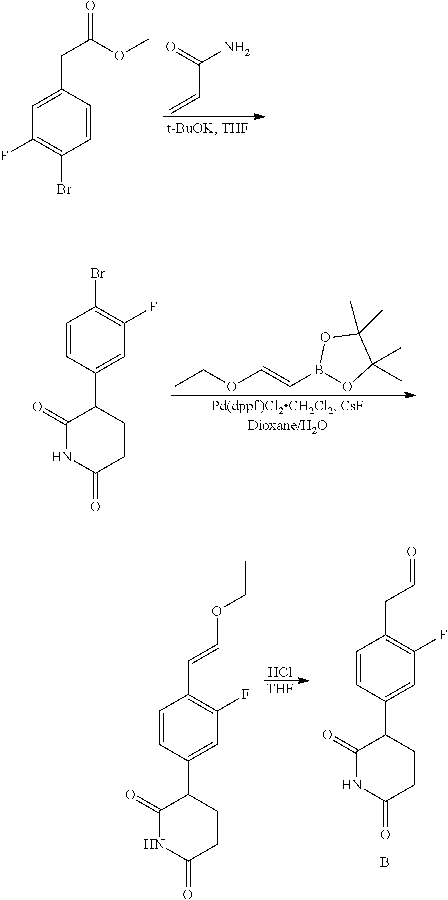

### Step 1—3-(4-bromo-3-fluorophenyl)piperidine-2,6-dione

To a solution of methyl 2-(4-bromo-3-fluorophenyl)acetate (4 g, 16.1 mmol, CAS #942282-41-9) and acrylamide (1.15 g, 16.2 mmol, 1.12 mL, CAS #79-06-1) in THF (40 mL) was added t-BuOK (2.00 g, 17.8 mmol) at 0° C. under nitrogen atmosphere. The reaction was then stirred at 50° C. for 2 hrs. On completion, the mixture was concentrated to give the crude product. The residue was purified by column chromatography (SiO₂, Petroleum ether:Ethyl acetate=10/1 to 1/1) to give the title compound (2.2 g, 48% yield) as a white solid. ¹H NMR (400 MHz, DMSO-d6) δ=10.88 (s, 1H), 7.74-7.59 (m, 1H), 7.36-7.27 (m, 1H), 7.06 (d, J=8.4 Hz, 1H), 3.93 (dd, J=4.8, 12.4 Hz, 1H), 2.74-2.59 (m, 1H), 2.55 (br t, J=3.6 Hz, 1H), 2.31-2.17 (m, 1H), 2.07-1.92 (m, 1H).

### Step 2—(E)-3-(4-(2-ethoxyvinyl)-3-fluorophenyl)piperidine-2,6-dione

A mixture of 3-(4-bromo-3-fluorophenyl)piperidine-2,6-dione (1.1 g, 3.84 mmol), (E)-2-(2-ethoxyvinyl)-4,4,5,5-tetramethyl-1,3,2-dioxaborolane (990 mg, 5.00 mmol, CAS #1201905-61-4), CsF (1.17 g, 7.69 mmol) and Pd(dppf)Cl₂·CH₂Cl₂ (314 mg, 384 μmol) in dioxane (10 mL) and H₂O (2 mL) was degassed and purged with N₂ three times. Then the mixture was stirred at 90° C. for 2 hrs under N₂ atmosphere. On completion, the mixture was concentrated to give the crude product. The residue was purified by column chromatography (SiO₂, Petroleum ether:Ethyl acetate=10/1 to 1/1) to give the title compound (600 mg, 54% yield) as a white solid. LC-MS (ESI+) m/z 278.2 (M+H)⁻; ¹H NMR (400 MHz, DMSO-d6) δ=10.83 (br s, 1H), 7.40 (t, J=8.0 Hz, 1H), 7.30-7.17 (m, 1H), 7.02 (br d, J=12.0 Hz, 1H), 6.96 (br d, J=8.0 Hz, 1H), 5.81 (d, J=13.2 Hz, 1H), 3.91 (q, J=7.2 Hz, 2H), 3.83 (br dd, J=4.8, 12.0 Hz, 1H), 2.70-2.59 (m, 1H), 2.21 (dq, J=4.0, 12.4 Hz, 1H), 2.08-1.99 (m, 2H), 1.25 (t, J=6.8 Hz, 3H).

### Step 3—2-(4-(2,6-dioxopiperidin-3-yl)-2-fluorophenyl)acetaldehyde

To a solution of (E)-3-(4-(2-ethoxyvinyl)-3-fluorophenyl)piperidine-2,6-dione (600 mg, 2.16 mmol) in THF (3 mL) was added HCl (6 M, 3.75 mL). The mixture was then stirred at 25° C. for 0.5 hours. On completion, the mixture was concentrated to give the crude product. The residue was purified by reverse-phase HPLC (0.1% FA condition) to give the title compound (200 mg, 31% yield) as a white solid. LC-MS (ESI+) m/z 250.0 (M+H)⁺.

### 4-Bromo-6-chloro-7-fluoro-N,N-dimethyl-1H-indole-2-carboxamide (Intermediate C)

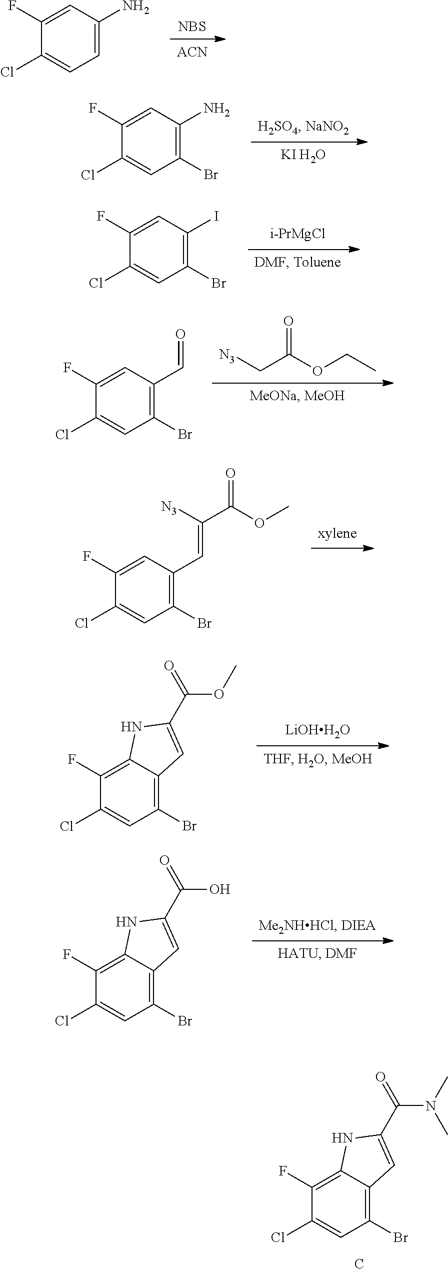

### Step 1—2-Bromo-4-chloro-5-fluoroaniline

To a solution of 4-chloro-3-fluoroaniline (100 g, 687 mmol, CAS #367-22-6) in ACN (1500 mL) was added NBS (122 g, 687 mmol, CAS #128-08-5) at 0° C. The mixture was then stirred at 25° C. for 12 h. On completion, the mixture was added H₂O (800 mL) and it was extracted with ethyl acetate (1000 mL×3), dried over Na₂SO₄ and evaporated. The residue was purified by column chromatography (SiO₂, Petroleum ether/Ethyl acetate=1/1 to 50/1) to give the title compound (83.1 g, 54% yield) as a white solid. ¹H NMR (400 MHz, CDCl₃) δ=7.43 (d, J=7.6 Hz, 1H), 6.57 (d, J=10.4 Hz, 1H), 4.19 (br s, 2H).

### Step 2—1-Bromo-5-chloro-4-fluoro-2-iodobenzene

A solution of 2-bromo-4-chloro-5-fluoroaniline (20.0 g, 89.1 mmol) in H₂O (640 mL) and H₂SO₄ (169 g, 1.69 mol, 92.1 mL) was cooled to 0° C. Next, a solution of NaNO₂ (6.76 g, 98.0 mmol) in H₂O (27 mL) was added and the mixture was stirred for 1 h. Then a solution of KI (19.4 g, 117 mmol) in H₂O (92 mL) was added and the reaction mixture was stirred at 25° C. for 12 h. On completion, ethyl acetate was added, and the phases were separated. The aqueous phase was extracted with ethyl acetate (50 mL×3). The combined organic phases are washed with 1 N NaOH (50 mL), 1 N sodium thiosulfate (50 mL), 1 N HCl (30 mL), NaHCO₃ (50 mL) and brine (5 mL), then dried over sodium sulfate, filtered, and concentrated under reduced pressure. The residue was purified by column chromatography (SiO₂. Petroleum ether:Ethyl acetate=1:0 to 25:1) to give the title compound (20.2 g, 68% yield) as a white solid. ¹H NMR (400 MHz, CDCl₃) δ=7.65 (dd, J=7.6, 8.8 Hz, 2H), 1.27 (s, 1H), 0.93-0.82 (m, 1H).

### Step 3—2-Bromo-4-chloro-5-fluorobenzaldehyde

A solution of the 1-bromo-5-chloro-4-fluoro-2-iodobenzene (20.2 g, 60.2 mmol) in toluene (180 mL) was cooled to −30° C. Then chloro(isopropyl)magnesium (2 M, 45.2 mL) was added slowly over 0.5 h at −30° C. and the mixture was stirred at this temperature for 1 h. Next, anhydrous DMF (17.6 g, 241 mmol, 18.5 mL) was added slowly over 30 min. Then the reaction mixture was wanned to 0° C. and stirred for 1 h. On completion, the reaction mixture was quenched by addition of NH₄Cl (60 mL) and extracted with EtOAc (60 mL×3). The combined organic layers were washed with brine (50 mL×3), dried over Na₂SO₄ and evaporated to give the title compound (14.5 g) as a white solid. ¹H NMR (400 MHz, CDCl₃) δ=10.25 (d, J=3.2 Hz, 1H), 7.76 (d, J=6.4 Hz, 1H), 7.71 (d, J=8.8 Hz, 1H).

### Step 4—Methyl (Z)-2-azido-3-(2-bromo-4-chloro-5-fluorophenyl)acrylate

To a solution of 2-bromo-4-chloro-5-fluorobenzaldehyde (14.5 g, 61.1 mmol) in MeOH (150 mL) was added dropwise NaOMe (44.0 g, 244 mmol, 30% solution) at −60° C. for 45 min. After addition, the mixture was stirred at this temperature for 1 h. and then ethyl 2-azidoacetate (31.5 g, 244 mmol, 28.0 mL, CAS #637-81-0) was added dropwise at −60° C. for 45 min. The resulting mixture was stirred at -60 to 25° C. for 12 h. On completion, the mixture was cooled to 0° C. and filtered. The filter cake was dried under reduced pressure. The residue was purified by column chromatography (SiO₂, Petroleum ether:Ethyl acetate=1:0-25:1) to give the title compound (8.70 g, 37% yield) as a white solid. ¹H NMR (400 MHz, CDCl₃) δ=8.09 (d, J=10.8 Hz, 1H), 7.66 (d, J=7.2 Hz, 1H), 7.13 (s, 1H), 3.96 (s, 3H).

### Step 5—Methyl 4-bromo-6-chloro-7-fluoro-1H-indole-2-carboxylate

To a solution of methyl (Z)-2-azido-3-(2-bromo-4-chloro-5-fluorophenyl)acrylate (3.17 g, 7.58 mmol) in xylene (30 mL) was reflux at 160° C. for 2 h. The mixture was then cooled to 0° C. and solid formed, which was filtered. The filter cake was dried under reduced pressure to give the title compound (667 mg) as a white solid. ¹H NMR (400 MHz, DMSO-d₆) δ=13.13 (br s, 11H), 7.52 (d, J=5.6 Hz, 1H), 7.09 (d, J=2.8 Hz, 1H), 3.90 (s, 3H).

### Step 6—4-Bromo-6-chloro-7-fluoro-1H-indole-2-carboxylic acid

To a solution methyl 4-bromo-6-chloro-7-fluoro-1H-indole-2-carboxylate (667 mg, 2.18 mmol) in THF (6 mL), H₂O (1.5 mL), and MeOH (1.5 mL) was added LiOH·H₂O (274 mg, 6.53 mmol), then the mixture stirred at 40° C. for 12 h. On completion, the reaction mixture was diluted by addition of H₂O (2 mL), and then extracted with DCM (6 mL×3). The combined aqueous phase was treated with HCl to adjusted pH=3˜4, then the mixture was extracted by DCM (4 mL×3). The combined organic phase was concentrated under reduced pressure to give the title compound (620 mg) as a white solid. ¹H NMR (400 MHz, DMSO-d₆) δ=13.12-12.76 (m, 1H), 7.50 (d, J=5.6 Hz, 1H), 7.03 (d, J=2.8 Hz, 1H).

### Step 7—4-Bromo-6-chloro-7-fluoro-N,N-dimethyl-1H-indole-2-carboxamide

To a solution of 4-bromo-6-chloro-7-fluoro-1H-indole-2-carboxylic acid (620 mg, 2.12 mmol) and N-methylmethanamine hydrochloride (259 mg, 3.18 mmol, CAS #506-59-2) in DMF (2 mL) was added DIEA (2.74 g, 21.2 mmol, 3.69 mL) and HATU (967 mg, 2.54 mmol). The mixture was then stirred at 0° C. for 2 h. On completion, the mixture was poured into H₂O (10 mL) and a solid precipitated. The solid was filtered and concentrated to give the title compound (617 mg, 91% yield) as a white solid. ¹H NMR (400 MHz, DMSO-d₆) δ=12.70 (br s, 1H), 7.46 (d, J=5.6 Hz, 1H), 6.79 (d, J=2.8 Hz, 1H), 3.29-3.05 (m, 10H).

### Tert-butyl 4-(3-methoxy-4-(4,4,5,5-tetramethyl-1,3,2-dioxaborolan-2-yl)phenyl)piperazine-1-carboxylate (Intermediate D)

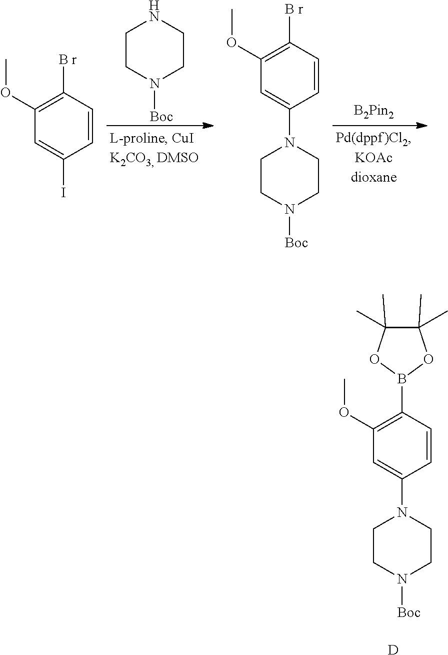

### Step 1—Tert-butyl 4-(4-bromo-3-methoxyphenyl)piperazine-1-carboxylate

A mixture of 1-bromo-4-iodo-2-methoxy-benzene (5.00 g, 15.9 mmol), tert-butyl piperazine-1-carboxylate hydrochloride (7.12 g, 31.9 mmol, CAS #57260-71-6). CuI (608 mg, 3.20 mmol), K₂CO₃ (6.62 g, 47.9 mmol) and (2S)-pyrrolidine-2-carboxylic acid (367 mg, 3.20 mmol) in DMSO (50 mL) was degassed and purged with N₂ three times. Then the mixture was stirred at 80° C. for 3 h under N₂ atmosphere. On completion, the reaction mixture was filtered and then diluted with H₂O (50 mL) and extracted with solvent ethyl acetate (150 mL). The combined organic layers were dried over Na₂SO₄, filtered, and concentrated under reduced pressure to give a residue. The residue was purified by flash silica gel chromatography (ISCO®; 40 g SepaFlash® Silica Flash Column, Eluent of 0˜15% Ethylacetate/Petroleum ether gradient @100 mL/min) to give the title compound (4 g, 67% yield) as a white solid. LC-MS (ESI+) m/z 372.3 (M+H)⁺; ¹H NMR (400 MHz, CHLOROFORM-d) δ ppm 7.38 (d, J=8.8 Hz, 1H), 6.48 (s, 1H), 6.41 (dd, J=2.4, 8.8 Hz, 1H), 3.88 (s, 3H), 3.65-3.53 (m, 4H), 3.21-3.06 (m, 4H), 1.49 (s, 9H).

### Step 2—Tert-butyl 4-(3-methoxy-4-(4,4,5,5-tetramethyl-1,3,2-dioxaborolan-2-yl)phenyl)piperazine-1-carboxylate

A mixture of tert-butyl 4-(4-bromo-3-methoxy-phenyl)piperazine-1-carboxylate (4.00 g, 10.7 mmol), 4,4,5,5-tetramethyl-2-(4,4,5,5-tetramethyl-1,3,2-dioxaborolan-2-yl)-1,3,2-dioxaborolane (3.01 g, 11.8 mmol), Pd(dppf)Cl₂ (788 mg, 1.08 mmol), and KOAc (3.17 g, 32.3 mmol) in dioxane (80 mL) was degassed and purged with N₂ three times. Then the mixture was stirred at 120° C. for 2 h under N₂ atmosphere. On completion, the reaction mixture was filtered and concentrated under reduced pressure to give a residue. The residue was purified by flash silica gel chromatography (ISCO®: 20 g SepaFlash® Silica Flash Column, Eluent of 0˜20% Ethyl acetate/Petroleum ether gradient @) 80 mL/min) to give the title compound (1.9 g, 41% yield) as a white solid. LC-MS (ESI+) m/z 419.3 (M+H); ¹H NMR (400 MHz, DMSO-d₆) δ ppm 7.40 (d, J=8.0 Hz, 1H), 6.46 (br d, J=8.4 Hz, 1H), 6.42 (s, 1H), 3.71 (s, 3H), 3.44 (br s, 4H), 3.24-3.15 (m, 4H), 1.42 (s, 9H), 1.23 (s, 12H).

### 1-(5-(4,4,5,5-tetramethyl-1,3,2-dioxaborolan-2-yl)-3,6-dihydropyridin-1(2H)-yl)ethan-1-one (Intermediate E)

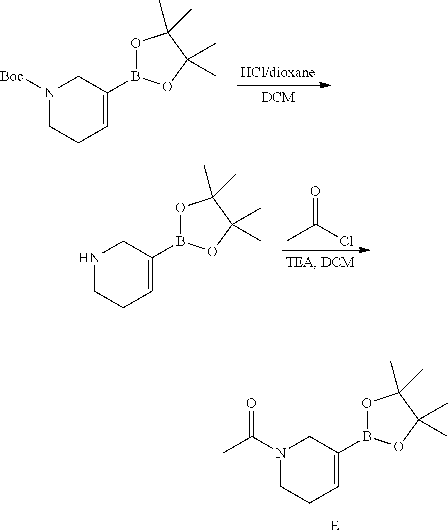

### Step 1—5-(4,4,5,5-Tetramethyl-1,3,2-dioxaborolan-2-yl)-1,2,3,6-tetrahydropyridine

To a solution of tert-butyl 5-(4,4,5,5-tetramethyl-1,3,2-dioxaborolan-2-yl)-3,6-dihydropyridine-1(2H)-carboxylate (30.0 g, 97.0 mmol, CAS #885693-20-9) in DCM (150 mL) was added HCl/dioxane (4 M, 150 mL), then the mixture was stirred at 20° C. for 1 h. After completion, the reaction mixture was concentrated under pressure to give the title compound (24.0 g, HCl) as a yellow solid. LC-MS (ESI+) m/z 210.3 (M+H)⁺.

### Step 2—1-(5-(4,4,5,5-Tetramethyl-1,3,2-dioxaborolan-2-yl)-3,6-dihydropyridin-1(2H)-yl)ethan-1-one

To a solution of 5-(4,4,5,5-tetramethyl-1,3,2-dioxaborolan-2-yl)-1,2,3,6-tetrahydropyridine (24.0 g, 97.7 mmol, HCl) in DCM (300 mL) was added DIEA (85.0 mL, 489 mmol) and acetyl chloride (10.5 mL, 147 mmol, CAS #75-36-5), then the mixture was stirred at 20° C. for 2 h. After completion, the reaction mixture was quenched by addition of water (500 mL) at 0° C., and then extracted with DCM (200 mL×2). The combined organic layers were washed with brine (500 mL), dried over anhydrous Na₂SO₄, filtered, and concentrated under reduced pressure to give a residue. The residue was triturated with EtOAc/PE=2:1 to afford the title compound (15.0 g, 59.7 mmol, 61.1% yield) as a yellow solid. ¹H NMR (400 MHz, DMSO-d₆) δ ppm 6.64-6.47 (m, 1H), 3.93 (s, 2H), 3.46 (td, J=5.6, 11.6 Hz, 2H), 2.24-2.09 (m, 2H), 2.00 (s, 3H), 1.21 (d, J=3.6 Hz, 12H).

### 6-(1-Acetyl-1,2,5,6-tetrahydropyridin-3-yl)-7-fluoro-4-(2-methoxy-4-(piperazin-1-yl)phenyl)-N,N-dimethyl-1H-indole-2-carboxamide (Intermediate F)

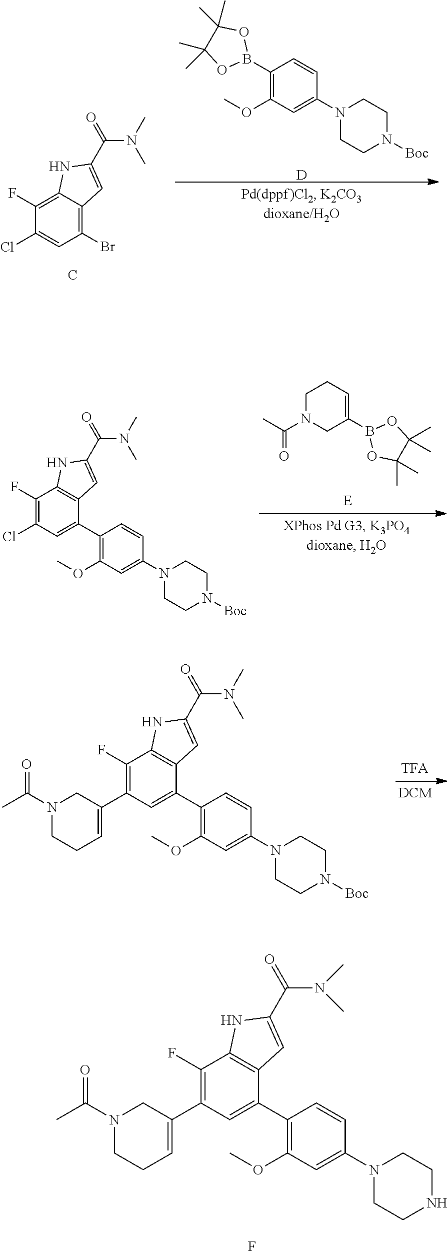

### Step 1—Tert-butyl 4-(4-(6-chloro-2-(dimethylcarbamoyl)-7-fluoro-1H-indol-4-yl)-3-methoxyphenyl)piperazine-1-carboxylate

A mixture of 4-bromo-6-chloro-7-fluoro-N,N-dimethyl-1H-indole-2-carboxamide (552 mg, 1.73 mmol, Intermediate C), tert-butyl 4-(3-methoxy-4-(4,4,5,5-tetramethyl-1,3,2-dioxaborolan-2-yl)phenyl)piperazine-1-carboxylate (759 mg, 1.81 mmol, Intermediate D), Pd(dppf)Cl₂ (126 mg, 173 μmol) and K₂CO₃ (716 mg, 5.18 mmol) in dioxane (6 mL) and H₂O (1.5 mL) was degassed and purged with N₂ three times. Then the mixture was stirred at 80° C. for 2 h under N₂ atmosphere. On completion, the reaction mixture was quenched by addition of H₂O (6 mL) and extracted with DCM (5 mL×3). Th combined organic layer was dried over Na₂SO₄ and evaporated. The residue was purified by column chromatography (SiO₂, Petroleum ether:Ethyl acetate˜DCM: Ethyl acetate=1:0-1:1) to give the title compound (830 mg, 90% yield) as a white solid. LC-MS (ESI+) m/z 531.2 (M+H)⁺.

### Step 2—Tert-butyl 4-(4-(6-(1-acetyl-1,2,5,6-tetrahydropyridin-3-yl)-2-(dimethylcarbamoyl)-7-fluoro-1H-indol-4-yl)-3-methoxyphenyl)piperazine-1-carboxylate

A mixture of tert-butyl 4-(4-(6-chloro-2-(dimethylcarbamoyl)-7-fluoro-1H-indol-4-yl)-3-methoxyphenyl)piperazine-1-carboxylate (780 mg, 1.47 mmol), 1-(5-(4,4,5,5-tetramethyl-1,3,2-dioxaborolan-2-yl)-3,6-dihydropyridin-1(2H)-yl)ethan-1-one (443 mg, 1.76 mmol, Intermediate E), XPhos Pd G3 (124 mg, 147 μmol) and K₃PO₄ (935 mg, 4.41 mmol) in dioxane (10 mL) and H₂O (2 mL) was degassed and purged with N₂ three times. Then the mixture was stirred at 80° C. for 2 h under N₂ atmosphere. On completion, the reaction mixture was quenched by addition of H₂O (6 mL) and extracted with DCM (5 mL×3), and the combined organic layer was dried over Na₂SO₄ and evaporated. The residue was purified by column chromatography (SiO₂, Petroleum ether:Ethyl acetate˜DCM: Ethyl acetate=1:0-1:2) to give the title compound (850 mg, 93% yield) as a brown solid. ¹H NMR (400 MHz, DMSO-d₆) δ=11.95 (br s, 1H), 7.21-7.17 (m, 1H), 6.83 (d, J=6.0 Hz, 1H), 6.69 (d, J=2.0 Hz, 1H), 6.61 (br d, J=8.4 Hz, 1H), 6.47 (t, J=2.8 Hz, 1H), 6.14-6.07 (m, 1H), 4.35-4.27 (m, 2H), 3.71 (s, 3H), 3.64-3.57 (m, 2H), 3.48 (br d, J=4.8 Hz, 4H), 3.25-3.00 (m, 10H), 2.40-2.31 (m, 2H), 2.07 (d, J=8.0 Hz, 3H), 1.43 (s, 9H).

### Step 3—6-(1-acetyl-1,2,5,6-tetrahydropyridin-3-yl)-7-fluoro-4-(2-methoxy-4-(piperazin-1-yl)phenyl)-N,N-dimethyl-1H-indole-2-carboxamide

A solution of tert-butyl 4-(4-(6-(1-acetyl-1,2,5,6-tetrahydropyridin-3-yl)-2-(dimethylcarbamoyl)-7-fluoro-1H-indol-4-yl)-3-methoxyphenyl)piperazine-1-carboxylate (300 mg, 484 μmol) in TFA (0.6 mL) and DCM (3 mL) was stirred at 25° C. for 3 h. On completion, the reaction mixture was quenched by addition of H₂O (2 mL), and then extracted with DCM (6 mL×3). The combined aqueous phase was treated with NaHCO₃ to adjusted pH=5˜6, then the mixture was extracted by DCM (4 mL×3). The combined organic phase was concentrated under reduced pressure to give the title compound (245 mg) as a yellow solid. LC-MS (ESI⁺) nm/z 520.3 (M+H)⁺.

### [1-[(4-Methoxyphenyl)methyl]-2,6-dioxo-3-piperidyl]trifluoromethanesulfonate (Intermediate G)

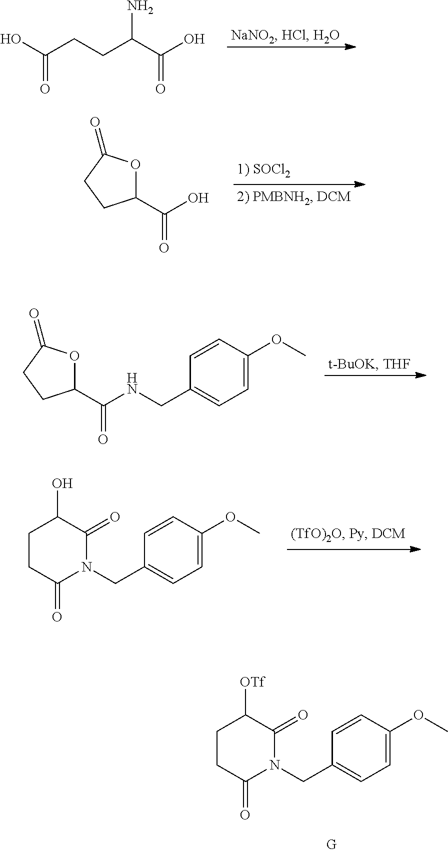

### Step 1—5—Oxotetrahydrofuran-2-carboxylic acid

To a solution of 2-aminopentanedioic acid (210 g, 1.43 mol, CAS #617-65-2) in H₂O (800 mL) and HCl (12 M, 210 mL) was added a solution of NaNO₂ (147 g, 2.13 mol) in H₂O (400 mL) at −5° C. The mixture was stirred at 15° C. for 12 hrs. On completion, the mixture was concentrated and then dissolved in EA (500 mL) and filtered and washed with EA (3×100 mL). The filtrate and washed solution were dried over Na₂SO₄, filtered, and concentrated in vacuo to give the title compound (200 g, crude) as yellow oil. ¹H NMR (400 MHz, CDCl₃) δ 6.43 (s, 1H), 5.02-4.95 (m, 1H), 2.67-2.38 (m, 4H)

### Step 2—N-[(4-methoxyphenyl)methyl]-5-oxo-tetrahydrofuran-2-carboxamide

To 5-oxotetrahydrofuran-2-carboxylic acid (120 g, 922 mmol) was added SOCl₂ (246 g, 2.07 mol) at 0° C. slowly. The mixture was stirred at 85° C. for 3 h, and then the mixture was stirred at 15° C. for 6 hrs. The mixture was concentrated in vacuo. The residue was dissolved in dry DCM (1 L) at 0° C. under N₂. After that a solution of Et₃N (187 g, 1.84 mol) and 4-methoxybenzylamine (101 g, 738 mmol) in DCM (400 mL) was added, then the mixture was stirred at 15° C. for 3 h. On completion, water (600 mL) was added and the mixture was extracted with DCM (3×300 mL). The combined organic phase was washed with 0.5 M HCl (500 mL), brine (500 mL), dried over with anhydrous sodium sulfate and filtered. The filtrate was concentrated in vacuo and the residue was purified by flash silica gel chromatography (PE:EA=1:1) to give the title compound (138 g, 60% yield) as a yellow solid. ¹H NMR (400 MHz, CDCl₃) δ 7.22-7.20 (d, J=8.0, 1H), 6.89-6.87 (d, J=8.0, 1H), 4.90-4.86 (m, 1H), 4.47-4.4.36 (m, 2H) 3.81 (s, 3H), 2.67-2.64 (m, 1H), 2.59-2.54 (m, 2H), 2.40-2.38 (m, 1H); LC-MS (ESI⁺) m/z 272.0 (M+Na)⁺.

### Step 3—3—Hydroxy-1-[(4-methoxyphenyl)methyl]piperidine-2,6-dione

A solution of N-[(4-methoxyphenyl)methyl]-5-oxo-tetrahydrofuran-2-carboxamide (138 g, 553 mmol) in anhydrous THF (1500 mL) was cooled to −78° C. Then, t-BuOK (62.7 g, 559 mmol) in a solution of anhydrous THF (1000 mL) was added dropwise slowly at −78° C. under nitrogen atmosphere. The resulting reaction mixture was stirred at −40° C. for 1 hr. On completion, the reaction mixture was quenched with saturated NH₄Cl solution (100 mL). The mixture was extracted with ethyl acetate (3×1500 mL). The combined organic layer was washed with brine (300 mL), dried over anhydrous sodium sulfate, filtered and the filtrate was concentrated in vacuo. The residue was purified by silica gel chromatography (PE:EA=1:1) to give the title compound (128 g, 92% yield) as a white solid. ¹H NMR (400 MHz, CDCl₃) δ 7.39-7.32 (m, 2H), 6.89-6.81 (m, 2H), 4.91 (s, 2H), 4.17-4.11 (m, 1H), 3.80 (s, 3H), 3.54 (s, 1H), 2.98-2.87 (m, 1H), 2.73-2.60 (m, 1H), 2.26-2.20 (m, 1H), 1.80 (dq, J=4.8, 13.1 Hz, 1H).

### Step 4—[1-[(4-Methoxyphenyl)methyl]-2,6-dioxo-3-piperidyl]trifluoromethanesulfonate

To a solution of 3-hydroxy-1-[(4-methoxyphenyl)methyl]piperidine-2, 6-dione (43.0 g, 173 mmol) and pyridine (27.3 g, 345 mmol) in DCM (500 mL) was added trifluoromethylsulfonyl trifluoromethanesulfonate (73.0 g, 258 mmol) dropwise at 0° C. The mixture was stirred at −10° C. for 1.5 hours under N₂. On completion, the mixture was concentrated in vacuo. The residue was purified by column chromatography on silica gel (PE:EA=20:1/8:1) to give the title compound (45.0 g, 68% yield) as a light, yellow gum. ¹H NMR (400 MHz, CDCl₃) δ 7.36 (d, J=8.4 Hz, 2H), 6.85-6.82 (m, 2H), 5.32-5.28 (m, 1H), 4.91 (s, 2H), 3.79 (s, 3H), 3.02-2.97 (m, 1H), 2.79-2.74 (m, 1H), 2.41-2.35 (m, 2H).

### 3-(4-bromo-3-methyl-2-oxo-2,3-dihydro-1H-benzo[d]imidazol-1-yl)piperidine-2,6-dione (Intermediate H)

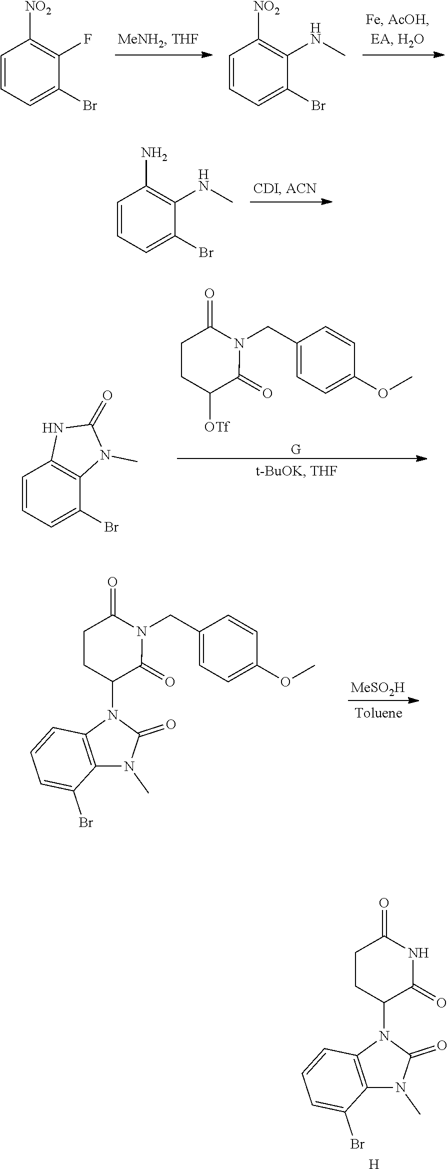

### Step 1—2-Bromo-N-methyl-6-nitro-aniline

To a solution of 1-bromo-2-fluoro-3-nitro-benzene (40.0 g, 181 mmol, CAS #58534-94-4) in THF (40 mL) was added MeNH₂ (2 M, 400 mL). The reaction mixture was stirred at 60° C. for 12 hours. On completion, the reaction mixture was poured into sat. NaHCO₃ (30 mL) and extracted with EA (3×200 mL). The combined organic layers were washed with brine (2×200 mL), dried with anhydrous Na₂SO₄, filtered and concentrated in vacuo to give the title compound (40.0 g, 95% yield) as red oil. LC-MS (ESI⁺) m/z 230.9 (M+H)⁺.

### Step 2—3-Bromo-N2-methyl-benzene-1,2-diamine

To a mixture of 2-bromo-N-methyl-6-nitro-aniline (23.0 g, 99.5 mmol) in EA (300 mL) and H₂O (10 mL) was added AcOH (100 mL). The mixture was warmed to 50° C. Then Fe (22.2 g, 398 mmol) was added to the reaction mixture and the mixture was heated to 80° C., about 4 hours. On completion, the reaction mixture was filtered and concentrated in vacuo. The residue was diluted with water (100 mL) and extracted with EA (3×200 mL). The combined organic layers were dried over Na₂SO₄, filtered and concentrated in vacuo to give the title compound (20.0 g, 99% yield) as red oil. ¹H NMR (400 MHz. DMSO-d₆) δ 6.73-6.70 (m, 1H), 6.68-6.60 (m, 2H), 5.02 (s, 2H), 3.67 (s, 1H), 2.58 (s, 3H).

### Step 3—4-Bromo-3-methyl-1H-benzimidazol-2-one

To a mixture of 3-bromo-N2-methyl-benzene-1,2-diamine (20.0 g, 99.4 mmol) in ACN (300 mL) was added CDI (32.2 g, 198 mmol). The reaction mixture was stirred at 85° C. for 12 hours under N₂ atmosphere. On completion, the reaction mixture was concentrated in vacuo. The reaction mixture was diluted with water (200 mL), where a solid precipitate was formed, which was filtered off. The solid was washed with water (1 L) and dried in vacuo to give the title compound (20.0 g, 88% yield) as white solid. ¹H NMR (400 MHz, DMSO-d₆) δ 11.17 (s, 111), 7.14 (dd, J=1.2, 8.0 Hz, 1H), 7.00-6.95 (m, 1H), 6.93-6.87 (m, 1H), 3.55 (s, 3H).

### Step 4—3-(4-Bromo-3-methyl-2-oxo-benzimidazol-1-yl)-1-[(4-methoxyphenyl)methyl]piperidine-2,6-dione

To a solution of 4-bromo-3-methyl-1H-benzimidazol-2-one (12.0 g, 52.8 mmol) in THF (300 mL) was added t-BuOK (7.12 g, 63.4 mmol). The reaction mixture was stirred at 0° C. for 0.5 hr. Subsequently, [1-[(4-methoxyphenyl)methyl]-2,6-dioxo-3-piperidyl]trifluoromethanesulfonate (20.1 g, 52.8 mmol, Intermediate G) in a solution of THF (100 mL) was added dropwise. The resulting reaction mixture was stirred at 20° C. for 0.5 hr under N₂. On completion, the reaction mixture was quenched with saturated NH₄Cl (100 mL) and extracted with ethyl acetate (200 mL). The combined organic layers were washed with brine (2×100 mL), dried over anhydrous sodium sulfate, filtered, the filtrate was concentrated in vacuo. The crude product was purified by reverse phase HPLC (0.1% FA condition) to give the title compound (13.3 g, 55% yield) as a yellow solid. ¹H NMR (400 MHz, CDCl₃) δ 7.38 (d, J=8.8 Hz, 2H), 7.22 (d, J=8.0 Hz, 1H), 6.84 (d, J=8.8 Hz, 2H), 6.80 (t, J=8.0 Hz, 1H), 6.48-6.40 (d, J=8.0 Hz, 1H), 5.22 (dd, J=5.2, 12.8 Hz, 1H), 5.04-4.93 (m, 2H), 3.81 (s, 3H), 3.80 (s, 3H), 3.12-2.98 (m, 1H), 2.93-2.77 (m, 1H), 2.62 (dq, J=4.4, 13.2 Hz, 1H), 2.20-2.17 (m, 1H).

### Step 5—3-(4-bromo-3-methyl-2-oxo-2,3-dihydro-1H-benzo[d]imidazol-1-yl)piperidine-2,6-dione

A mixture of 3-(4-bromo-3-methyl-2-oxo-benzimidazol-1-yl)-1-[(4-methoxyphenyl)methyl]piperidine -2,6-dione (13.3 g, 29.0 mmol) in a mixed solvent of Tol. (80 mL) and methane sulfonic acid (40 mL) was degassed and purged with N₂ for 3 times, and then the mixture was stirred at 120° C. for 2 h under N₂ atmosphere. On completion, the reaction mixture was concentrated in vacuo to remove toluene. To the residue was added 200 mL of ice water, and then white solid precipitate formed. The mixture was filtered and the filtered cake was collected and dried over in vacuo to give the title compound (7.30 g, 74% yield) as white solid. ¹H NMR (400 MHz, DMSO-d₆) δ 11.13 (s, 1H), 7.25 (d, J=8.0 Hz, 1H), 7.17 (d, J=8.0 Hz, 1H), 7.05-6.93 (m, 1H), 5.41 (dd, J=5.2, 12.8 Hz, 1H), 3.64 (s, 3H), 2.96-2.83 (m, 1H), 2.78-2.59 (m, 2H), 2.08-2.00 (m, 1H).

### 5-Bromo-3-methyl-1H-benzimidazol-2-one (Intermediate I)

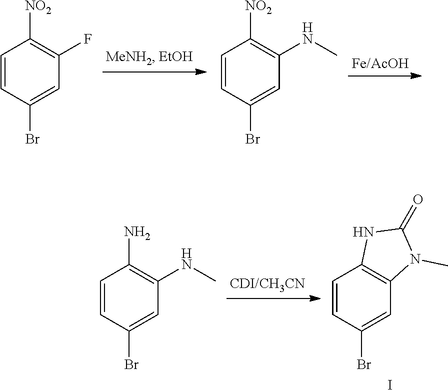

### Step 1—5-Bromo-N-methyl-2-nitro-aniline

4-bromo-2-fluoro-1-nitro-benzene (230 g, 1.05 mol, CAS #321-23-3) was added to a solution of methylamine in tetrahydrofuran (2 M, 1.51 L). The mixture was stirred at 15° C. for 10 minutes. On completion, the mixture was diluted with H₂O (250 mL) and extracted with EtOAc (3×300 mL). The combined organic layers were washed with brine (300 mL), dried over Na₂SO₄, filtered and concentrated in vacuo to give the title compound (200 g, 83% yield) as a yellow solid. ¹H NMR (400 MHz, DMSO-d₆) δ 8.22 (s, 1H), 7.98 (d, J=9.2 Hz, 1H), 7.16 (d, J=1.6 Hz, 1H), 6.82 (dd, J=8.4, 1.6 Hz, 1H), 2.95 (d, J=4.8 Hz, 3H).

### Step 2—4-Bromo-N2-methyl-benzene-1,2-diamine

To a mixture of 5-bromo-N-methyl-2-nitro-aniline (200 g, 865 mmol) in EtOAc (1 L) and H₂O (500 mL) was added AcOH (1.00 L). The mixture was warmed to 50° C., and then Fe (174 g, 3.11 mol) was added to the reaction mixture. After that, the reaction mixture was stirred at 80° C. for 6 hours. On completion, the mixture was filtered through celite. The filtrate was concentrated in vacuo and the residue was diluted with H₂O (250 mL) and extracted with EtOAc (3×300 mL). The combined organic layers were washed with aq. NaHCO₃ and brine (300 mL), dried over Na₂SO₄, filtered and concentrated in vacuo. The residue was purified by flash silica gel chromatography to give the title compound (130 g, 75% yield) as black oil. ¹H NMR (400 MHz. DMSO-d₆) δ 6.55-6.52 (m, 1H), 6.48-6.45 (m, 1H), 6.43-6.42 (m, 1H), 4.89-4.88 (m, 1H), 4.61 (s, 2H), 2.70 (d, J=4.0 Hz, 3H).

### Step 3—5-Bromo-3-methyl-1H-benzimidazol-2-one

To a solution of 4-bromo-N2-methyl-benzene-1,2-diamine (110 g, 547 mmol) in CH₃CN (1.3 L) was added CDI (177 g, 1.09 mol). The mixture was stirred at 80° C. for 6 hours under N₂. On completion, the mixture was concentrated in vacuo. The mixture was diluted with H₂O (1.0 L) and filtered. The filter cake was washed with water (3×200 mL) and dried in vacuo to give the title compound (106 g, 85% yield) as a white solid. ¹H NMR (400 MHz, DMSO-d₆) δ 11.00 (s, 111), 7.33 (s, 1H), 7.13 (d, J=8.0 Hz, 1H), 6.92 (d, J=8.0 Hz, 1H), 3.27 (s, 3H).

### 3-(5-bromo-3-methyl-2-oxo-benzimidazol-1-yl)piperidine-2,6-dione (Intermediate J)

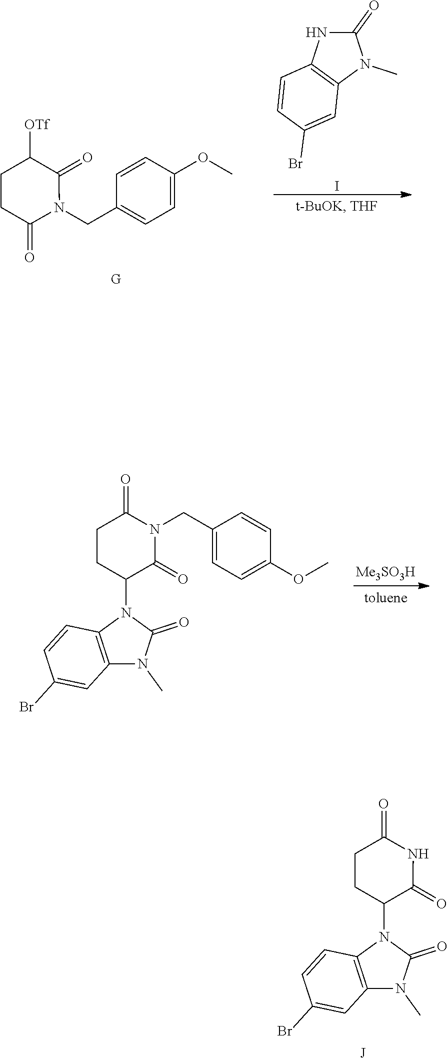

### Step 1—3-(5-Bromo-3-methyl-2-oxo-benzimidazol-1-yl)-1-[(4-methoxyphenyl)methyl]piperidine-2,6-dione

To a solution of 5-bromo-3-methyl-1H-benzimidazol-2-one (4.90 g, 21.6 mmol, Intermediate I) in THF (300 mL) was added t-BuOK (3.63 g, 32.3 mmol) at 0° C. The mixture was stirred at 0-10° C. for 1 hour under N₂. Then a solution of [1-[(4-methoxyphenyl)methyl]-2, 6-dioxo-3-piperidyl]trifluoromethanesulfonate (9.87 g, 25.9 mmol. Intermediate G) in THF (100 mL) was added to the reaction mixture at 0-10° C. during 30 minutes. The mixture was stirred at 0-10° C. for 30 minutes under N₂. An additional solution of [1-[(4-methoxyphenyl)methyl]-2, 6-dioxo-3-piperidyl]trifluoromethanesulfonate (2.47 g, 6.47 mmol) in THF (20 mL) was added to the reaction mixture at 0-10° C. dropwise. The mixture was then stirred at 0-10° C. for another 30 minutes under N₂. On completion, the reaction was quenched water (400 mL) and extracted with EA (3×200 mL). The combined organic layer was concentrated in vacuo. The residue was triturated with EA (80 mL) and filtered. The filter cake was collected and dried in vacuo to give the title compound (6.70 g, 67% yield) as light yellow solid. The filtrate was also concentrated in vacuo and the residue was purified by column chromatography to give another batch title compound (1.80 g, 18% yield) as light yellow solid. ¹H NMR (400 MHz, DMSO-d₆) δ 7.47 (d, J=1.6 Hz, 1H), 7.21-7.16 (m, 3H), 7.01 (d, J=8.0 Hz, 1H), 6.85 (d, J=8.8 Hz, 2H), 5.55-5.51 (m, 1H), 4.84-4.73 (m, 2H), 3.72 (s, 3H), 3.33 (s, 3H), 3.04-3.00 (m, 1H), 2.83-2.67 (m, 2H), 2.07-2.05 (m, 1H).

### Step 2—3-(5-Bromo-3-methyl-2-oxo-benzimidazol-1-yl)piperidine-2,6-dione

To a mixture of 3-(5-bromo-3-methyl-2-oxo-benzimidazol-1-yl)-1-[(4-methoxyphenyl)methyl]piperidine-2,6-dione (8.50 g, 18.6 mmol) in toluene (50 mL) was added methanesulfonic acid (33.8 g, 351 mmol, 25 mL) at room temperature (15° C.). The mixture was stirred at 120° C. for 2 hours. On completion, the reaction mixture was cooled to room temperature and concentrated in vacuo. The residue was poured into ice/water (200 mL) and extracted with EA (3×100 mL). The combined organic layer was washed with brine (50 mL), dried over Na₂SO₄, filtered and concentrated in vacuo. The residue was triturated with EA (80 mL) and filtered. The filtrate cake was collected and dried in vacuo to give the title compound (4.20 g, 67% yield) as off-white solid. ¹H NMR (400 MHz, DMSO-d₆) δ 11.12 (s, 1H), 7.47 (d, J=2.0 Hz, 1H), 7.22 (d, J=8.4 Hz, 1H), 7.10 (d, J=8.4 Hz, 1H), 5.40-5.35 (m 1H), 2.34 (s, 3H), 2.92-2.88 (m, 1H), 2.71-2.60 (m, 2H), 2.03-1.99 (m, 1H).

### 3-(1-(2,6-Dioxopiperidin-3-yl)-3-methyl-2-oxo-2,3-dihydro-1H-benzo[d]imidazol-5-yl)propanal (Intermediate K)

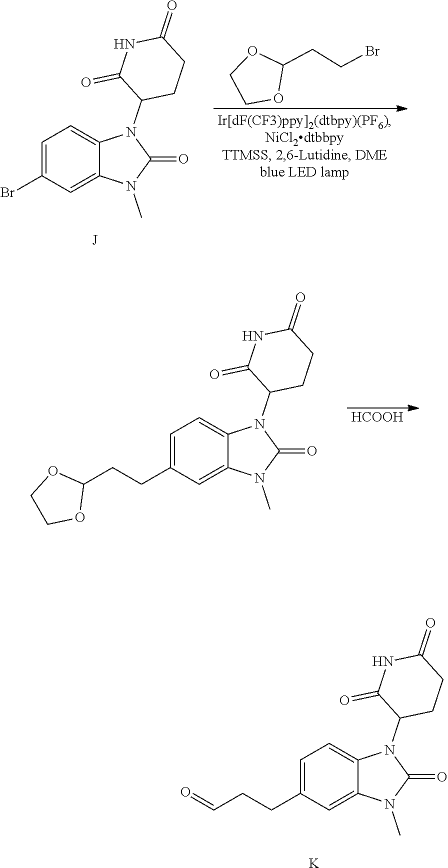

### Step 1—3-(1-(2,6-Dioxopiperidin-3-yl)-3-methyl-2-oxo-2,3-dihydro-1H-benzo[d]imidazol-5-yl)propanal

A solution of 3-(5-bromo-3-methyl-2-oxo-2,3-dihydro-1H-benzo[d]imidazol-1-yl)piperidine-2,6-dione (20 g, 59.1 mmol, Intermediate J), 2-(2-bromoethyl)-1,3-dioxolane (13.9 g, 76.8 mmol, 9.22 mL, CAS #18742-02-4), Ir[dF(CF₃)ppy]₂(dtbpy)(PF₆) (1.33 g, 1.18 mmol), NiCl₂·dtbbpy (706 mg, 1.77 mmol). TTMSS (17.6 g, 70.9 mmol, 21.9 mL), and 2,6-dimethylpyridine (57.0 g, 532 mmol, 62.0 mL) in DME (500 mL) was stirred and irradiated with a 4×50 W [455 nm] blue LED lamp (3 cm away), with cooling water to keep the reaction temperature at 25° C. for 14 h. On completion, the reaction mixture was concentrated in vacuo to give the crude residue. The residue was purified by column chromatography (SiO₂, Petroleum ether:Ethyl acetate=1/0 to 0/1) to give the title compound (12 g, 53% yield) as a white solid. LC-MS (ESI⁺) m/z 360.0 (M+H)⁺.

### Step 2—3-(1-(2,6-dioxopiperidin-3-yl)-3-methyl-2-oxo-2,3-dihydro-1H-benzo[d]imidazol-5-yl)propanal

A solution of 3-(5-(2-(1,3-dioxolan-2-yl)ethyl)-3-methyl-2-oxo-2,3-dihydro-1H-benzo[d]imidazol-1-yl)piperidine-2,6-dione (200 mg, 557 μmol) in HCOOH (2 mL) was stirred at 40° C. for 2 h. On completion, the solution was concentrated in vacuo to give the title compound (263 mg) as a brown solid. LC-MS (ESI⁺) m/z 316.1 (M+H)⁺.

### Ethyl 7-bromo-5-chloro-4-fluoro-benzofuran-2-carboxylate (Intermediate L)

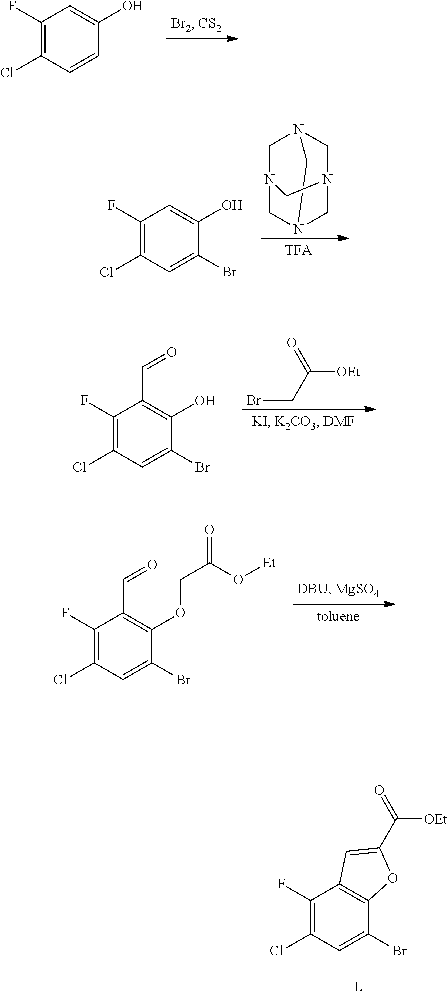

### Step 1—2-Bromo-4-chloro-5-fluorophenol

To a solution of 4-chloro-3-fluoro-phenol (100 g, 682.38 mmol, CAS #348-60-7) in CS₂ (1000 mL) was degassed and purged with nitrogen. The mixture was then cooled to 0° C. before Br₂ (109 g, 682 mmol, CAS #7726-95-6) was added. The mixture was then stirred at 20° C. for 12 h. On completion, the mixture was concentrated under reduced pressure then was quenched with aqueous NaClO. The residue was diluted in EtOAc (500 mL) and poured into 500 mL saturated Na₂S₂O₃ solution, then extracted with EtOAc (2×500 mL). The combined organic layer was washed by brine (2×500 mL), dried over Na₂SO₄, filtered and concentrated. The residue was purified by column chromatography (SiO₂, Petroleum ether) to afford the title compound (128 g, 80% yield) as an orange liquid. LC-MS (ESI⁺) m/z 222.8 (M−1)⁺; ¹H NMR (400 MHz, DMSO-d₆) δ=11.05 (br s, 1H), 7.73 (d, J=8.0 Hz, 1H), 6.91 (d, J=10.8 Hz, 1H).

### Step 2—3-Bromo-5-chloro-6-fluoro-2-hydroxybenzaldehyde

A solution of 2-bromo-4-chloro-5-fluoro-phenol (128 g, 511 mmol) in TFA (510 mL) was degassed and purged with nitrogen before cooling to 0° C. Then 1,3,5,7-tetrazatricyclo[3,3,1,13,7]decane (83.2 g, 594 mmol, CAS #100-97-0) was added to the mixture at 20° C. The mixture was then refluxed at 80° C. for 12 h. On completion, water (200 mL) was added to the mixture at 0° C., followed by 50% H₂SO₄ (200 mL). Then the mixture was stirred for 1 h and filtered. The filter cake was dried under reduced pressure. The crude residue was purified by column chromatography (SiO₂, Petroleum ether) to afford the title compound (27 g, 19% yield) as a light yellow solid. ¹H NMR (400 MHz, DMSO-d₆) δ=11.78 (br s, 1H), 10.16 (s, 1H), 8.22 (d, J=8.0 Hz, 1H).

### Step 3—Ethyl 2-(6-bromo-4-chloro-3-fluoro-2-formylphenoxy)acetate

To a solution of 5-bromo-3-chloro-2-fluoro-6-hydroxy-benzaldehyde (27 g, 106.53 mmol) in DMF (150 mL) was added potassium iodide (3.54 g, 21.31 mmol, CAS #7681-11-0) and potassium carbonate (29.5 g, 213 mmol, CAS #584-08-7). The mixture was purged with nitrogen before ethyl 2-bromoacetate (21.35 g, 127.83 mmol, 14.15 mL, CAS #105-36-2) was added. The mixture was then stirred at 40° C. for 1 h. On completion, the mixture was filtered and quenched with 200 mL HCl (2 M in water). The mixture was then extracted with EtOAc (2×200 mL). The combined organic layer was washed with brine (2×200 mL), dried over Na₂SO₄, filtered and concentrated. The residue was purified by flash silica gel chromatography (ISCO®; 120 g SepaFlash® Silica Flash Column, Eluent of 3˜5% Ethyl acetate/Petroleum ether gradient @ 200 mL/min) to afford the title compound (23 g, 64% yield) as a pale yellow solid. ¹H NMR (400 MHz, DMSO-d₆) δ=10.28 (s, 1H), 8.33 (d, J=8.0 Hz, 1H), 4.84 (s, 2H), 4.17 (d, J=7.2 Hz, 2H), 1.22 (t, J=7.2 Hz, 3H).

### Step 4—Ethyl 7-bromo-5-chloro-4-fluorobenzofuran-2-carboxylate

To a solution of ethyl 2-(6-bromo-4-chloro-3-fluoro-2-formyl-phenoxy)acetate (17.5 g, 46.4 mmol) and MgSO₄ (8.38 g, 69.6 mmol. CAS #22189-08-8) in toluene (400 mL) was purged with nitrogen before DBU (15.5 g, 102 mmol, 15.4 mL, CAS #41015-70-7) was added. The mixture was then heated to 120° C. and stirred for 2 h. On completion, the mixture was quenched with 1 N HCl (200 mL) and extracted with EtOAc (400 mL). The organic layer was washed with brine (2×250 mL), dried over Na₂SO₄, filtered and concentrated. The residue was purified by flash silica gel chromatography (ISCO®; 120 g SepaFlash® Silica Flash Column, Eluent of 0˜3% Ethyl acetate/Petroleum ether gradient @80 mL/min) to afford the title compound (7.3 g, 44% yield) as an off-white solid. ¹H NMR (400 MHz, DMSO-d₆) 5=8.05 (d, J=6.4 Hz, 1H), 7.98 (s, 1H), 4.40 (q, J=7.2 Hz, 2H), 1.35 (t, J=7.2 Hz, 3H).

### Tert-butyl 4-(4-(5-chloro-2-(dimethylcarbamoyl)-4-fluorobenzofuran-7-yl)phenyl)piperazine-1-carboxylate (Intermediate M)

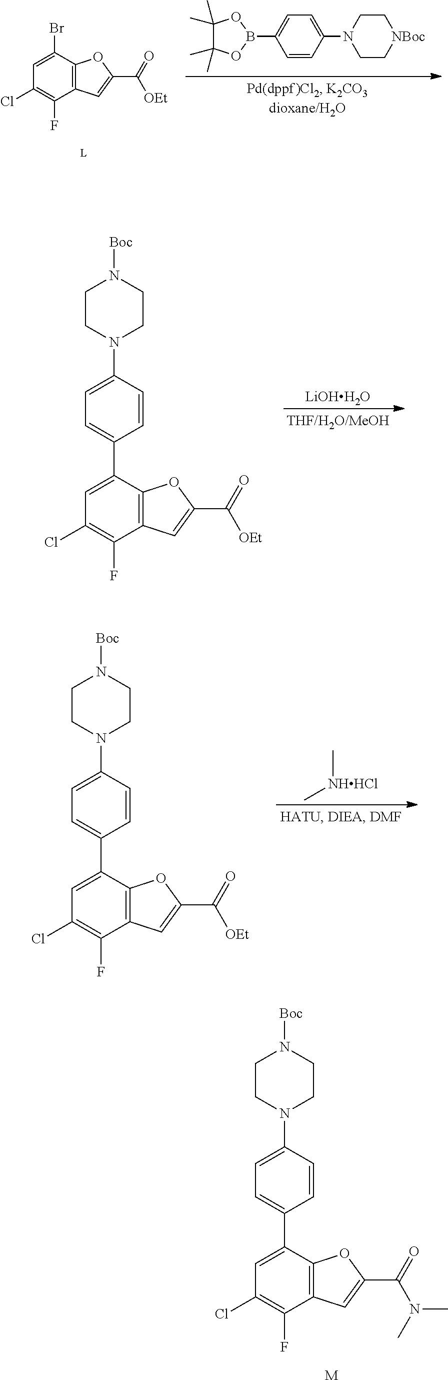

### Step 1—Tert-butyl 4-(4-(5-chloro-2-(ethoxycarbonyl)-4-fluorobenzofuran-7-yl)phenyl)piperazine-1-carboxylate

To a solution of ethyl 7-bromo-5-chloro-4-fluoro-benzofuran-2-carboxylate (3.80 g, 11.8 mmol, Intermediate L), tert-butyl 4-[4-(4,4,5,5-tetramethyl-1,3,2-dioxaborolan-2-yl)phenyl]piperazine-1-carboxylate (4.82 g, 12.4 mmol, CAS #470478-90-1) in dioxane (30 mL) and H₂O (10 mL) was added Pd(dppf)Cl₂ (864 mg, 1.18 mmol) and K₂CO₃ (4.90 g, 35.4 mmol) under nitrogen atmosphere. Then the reaction was stirred at 80° C. for 4 h under nitrogen atmosphere. On completion, the reaction mixture was diluted with water (60 mL) and extracted with ethyl acetate (3×50 mL). The combined organic layers were dried over anhydrous Na₂SO₄, filtered and the filtrate was concentrated in vacuo to give the crude residue. The residue was purified by column chromatography (SiO₂, PE:EA=100/1 to 0/1) to give the title compound (5 g, 84% yield) as a white solid. LC-MS (ESI⁺) m/z 503.0 (M+H)⁺.

### Step 2—7-(4-(4-(Tert-butoxycarbonyl)piperazin-1-yl)phenyl)-5-chloro-4-fluorobenzofuran-2-carboxylic acid

To a solution of tert-butyl 4-(4-(5-chloro-2-(ethoxycarbonyl)-4-fluorobenzofuran-7-yl)phenyl)piperazine-1-carboxylate (4.9 g, 9.74 mmol) in THF (21 mL), H₂O (7 mL) and MeOH (7 mL) was added LiOH·H₂O (4.09 g, 97.4 mmol). The mixture was then stirred at 40° C. for 2 h. On completion, HCl (2 N) was added to the reaction mixture until the pH 6. Then the mixture was diluted with water (45 mL) and extracted with ethyl acetate (3×45 mL). The combined organic layers were dried over anhydrous sodium sulfate, filtered and the filtrate was concentrated in vacuo to give the title compound (4.2 g) as a yellow solid. LC-MS (ESI⁺) m/z 475.0 (M+H)⁺.

### Step 3—Tert-butyl 4-(4-(5-chloro-2-(dimethylcarbamoyl)-4-fluorobenzofuran-7-yl)phenyl)piperazine-1-carboxylate

To a solution of 7-(4-(4-(tert-butoxycarbonyl)piperazin-1-yl)phenyl)-5-chloro-4-fluorobenzofuran-2-carboxylic acid (2.3 g, 4.84 mmol) and N-methylmethanamine hydrochloride (789 mg, 9.69 mmol, CAS #506-59-2) in DMF (20 mL) was added HATU (3.68 g, 9.69 mmol) and DIEA (6.26 g, 48.4 mmol). The mixture was then stirred at 25° C. for 1 hr. On completion, the mixture was diluted with H₂O (100 mL) and solid formed. Then the reaction mixture was filtered and the filter cake was dried in vacuo to give the title compound (3 g) as a yellow solid. LC-MS (ESI⁺) m/z 502.2 (M+H)⁺.

### 3-(1H-pyrazol-1-yl)-1-(5-(4,4,5,5-tetramethyl-1,3,2-dioxaborolan-2-yl)-3,6-dihydropyridin-1(2H)-yl)propan-1-one (Intermediate N)

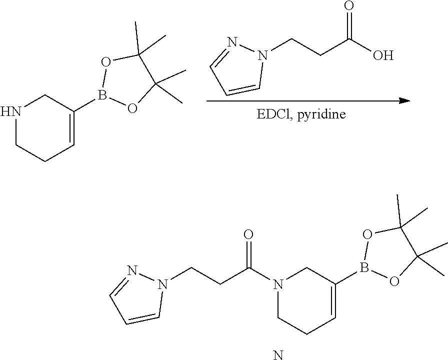

### Step 1—3-(1H-pyrazol-1-yl)-1-(5-(4,4,5,5-tetramethyl-1,3,2-dioxaborolan-2-yl)-3,6-dihydropyridin-1(2H)-yl)propan-1-one

To a solution of 3-(1H-pyrazol-1-yl)propanoic acid (25 g, 178 mmol, CAS #89532-73-0) in anhydrous pyridine (200 mL) was added EDCI (41.0 g, 214 mmol). Then 5-(4,4,5,5-tetramethyl-1,3,2-dioxaborolan-2-yl)-1,2,3,6-tetrahydropyridine (48.2 g, 196 mmol, HCl, synthesized via Step 1 of Intermediate E) was added, and the reaction was stirred at 20° C. for 5 h under nitrogen atmosphere. On completion, the mixture was concentrated to remove pyridine and give a crude residue. The residue was purified by column chromatography (SiO₂, dichloromethane: ethyl acetate=10/1 to 1/1) to give the title compound (43 g, 73% yield) as a white solid. LC-MS (ESI⁺) m/z 332.0 (M+H)⁺. ¹H NMR (400 MHz, DMSO-d6) δ=7.69 (s, 1H), 7.49-7.29 (m, 1H), 6.66-6.40 (m, 1H), 6.19 (s, 1H), 4.32 (br t, J=6.8 Hz, 2H), 4.00-3.85 (m, 2H), 3.55-3.38 (m, 2H), 2.88 (q, J=6.8 Hz, 2H), 2.25-2.05 (m, 2H), 1.21 (s, 12H).

### 5-(1-(3-(1H-pyrazol-1-yl)propanoyl)-1,2,5,6-tetrahydropyridin-3-yl)-4-fluoro-N,N-dimethyl-7-(4-(piperazin-1-yl)phenyl)benzofuran-2-carboxamide (Intermediate O)

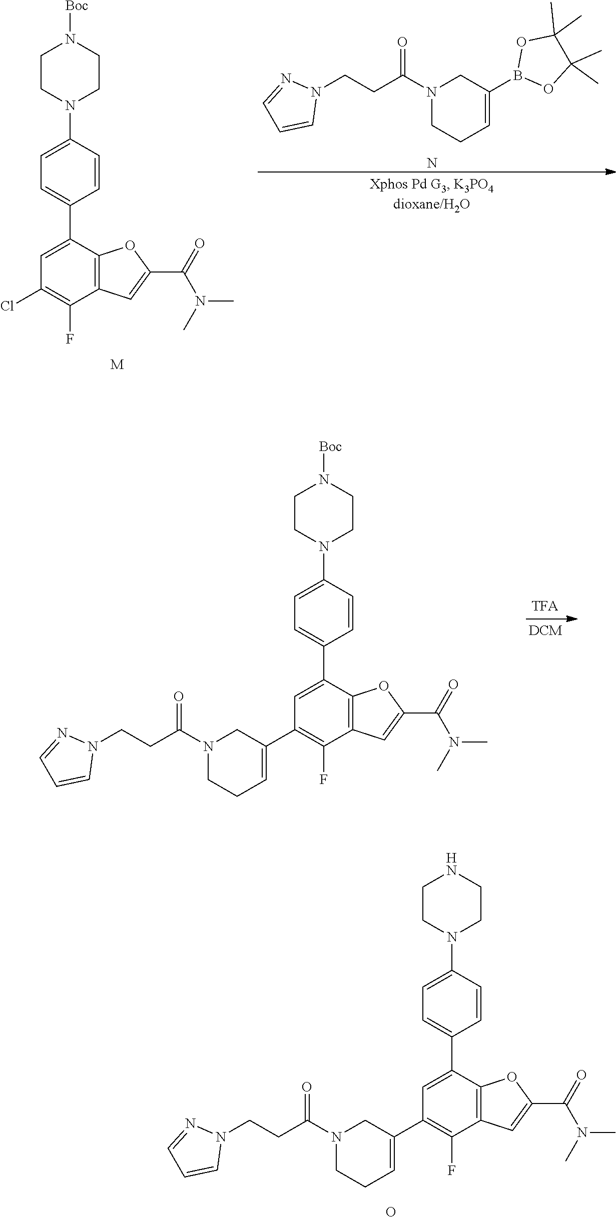

### Step 1—Tert-butyl 4-(4-(5-(1-(3-(1H-pyrazol-1-yl)propanoyl)-1,2,5,6-tetrahydropyridin-3-yl)-2-(dimethylcarbamoyl)-4-fluorobenzofuran-7-yl)phenyl)piperazine-1-carboxylate

To a solution of 3-(1H-pyrazol-1-yl)-1-(5-(4,4,5,5-tetramethyl-1,3,2-dioxaborolan-2-yl)-3,6-dihydropyridin-1(2H)-yl)propan-1-one (2.64 g, 7.97 mmol, Intermediate N) and tert-butyl 4-(4-(5-chloro-2-(dimethylcarbamoyl)-4-fluorobenzofuran-7-yl)phenyl)piperazine-1-carboxylate (4.00 g, 7.97 mmol, Intermediate M) in dioxane (30 mL) and H₂O (10 mL) was added and X Phos Pd G3 (674 mg, 796 μmol) and K₃PO₄ (5.07 g, 23.9 mmol) under nitrogen atmosphere. Then the reaction was stirred at 80° C. for 2 h under nitrogen atmosphere. On completion, the reaction mixture was diluted with water (60 mL) and extracted with ethyl acetate (3×50 mL). The combined organic layers were dried over anhydrous Na₂SO₄, filtered and the filtrate was concentrated in vacuo to give the crude residue. The residue was purified by column chromatography (SiO₂, PE:EA=100/1 to 0/1) to give the title compound (3.5 g, 65% yield) as a white solid. LC-MS (ESI⁺) m/z 671.4 (M+H)⁺.

### Step 2—5-(1-(3-(1H-pyrazol-1-yl)propanoyl)-1,2,5,6-tetrahydropyridin-3-yl)-4-fluoro-N,N-dimethyl-7-(4-(piperazin-1-yl)phenyl)benzofuran-2-carboxamide

To a solution of tert-butyl 4-(4-(5-(1-(3-(1H-pyrazol-1-yl)propanoyl)-1,2,5,6-tetrahydropyridin-3-yl)-2-(dimethylcarbamoyl)-4-fluorobenzofuran-7-yl)phenyl)piperazine-1-carboxylate (0.5 g, 745 μmol) in DCM (10 mL) was added TFA (5 mL). The mixture was then stirred at 25° C. for 2 h. On completion, the reaction mixture was concentrated in vacuo to give the crude residue. The crude residue was purified by reverse phase HPLC (0.1% FA condition) to give the title compound (0.2 g, 47% yield, FA) as a yellow solid. LC-MS (ESI⁺) m/z 571.4 (M+H)⁺.

### Methyl 6-bromo-4-chloro-7-fluoro-1H-indole-2-carboxylate (Intermediate P)

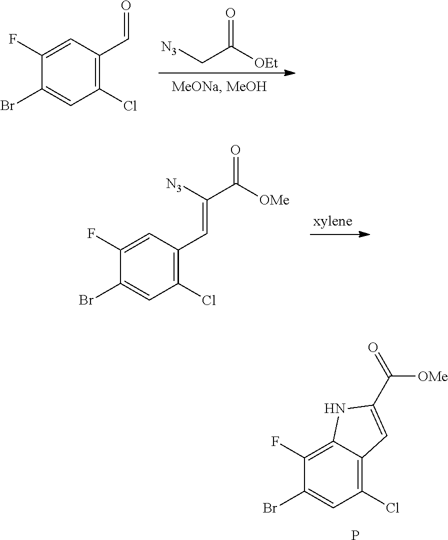

### Step 1—Methyl 2-azido-3-(4-bromo-2-chloro-5-fluorophenyl) acrylate

A solution of 4-bromo-2-chloro-5-fluorobenzaldehyde (18 g, 76 mmol) and ethyl 2-azidoacetate (39.1 g, 303 mmol) in MeOH (250 mL) was added to a mixture of NaOMe (5.4 M, 56.1 mL) in MeOH (250 mL) dropwise at −10° C. The mixture was warmed up to 20° C. and stirred for 12 h. On completion, the reaction mixture was poured into NH₄Cl (600 mL) at 0° C., then filtered and the solid was dried in vacuo. The residue was purified by column chromatography (SiO₂, Petroleum ether:Ethyl acetate=100/1 to 4/1) to give the title compound (17 g, 67% yield) as a white solid. ¹H NMR (400 MHz, DMSO-d₆) δ=8.21 (d, J=10.4 Hz, 1H), 8.06 (d, J=6.8 Hz, 1H), 7.01 (s, 1H), 3.94 (s, 3H).

### Step 2—Methyl 6-bromo-4-chloro-7-fluoro-1H-indole-2-carboxylate

Methyl 2-azido-3-(4-bromo-2-chloro-5-fluorophenyl) acrylate (15 g, 44.84 mmol) was added to xylene (500 mL) and the reaction was refluxed for 3 h. On completion, the reaction mixture was cooled to rt, filtered and the filter cake was dried in vacuo to give the title compound (11 g, 80% yield) as a white solid. ¹H NMR (400 MHz, DMSO-d₆) δ=13.09 (s, 1H), 7.44 (d, J=4.4 Hz, 1H), 7.16 (s, 1H), 3.90 (s, 3H).

### (R)-6-(1-(3-(1H-pyrazol-1-yl)propanoyl)piperidin-3-yl)-4-(4-(4-(tert-butoxycarbonyl)piperazin-1-yl)phenyl)-7-fluoro-1H-indole-2-carboxylic acid (Intermediate Q)

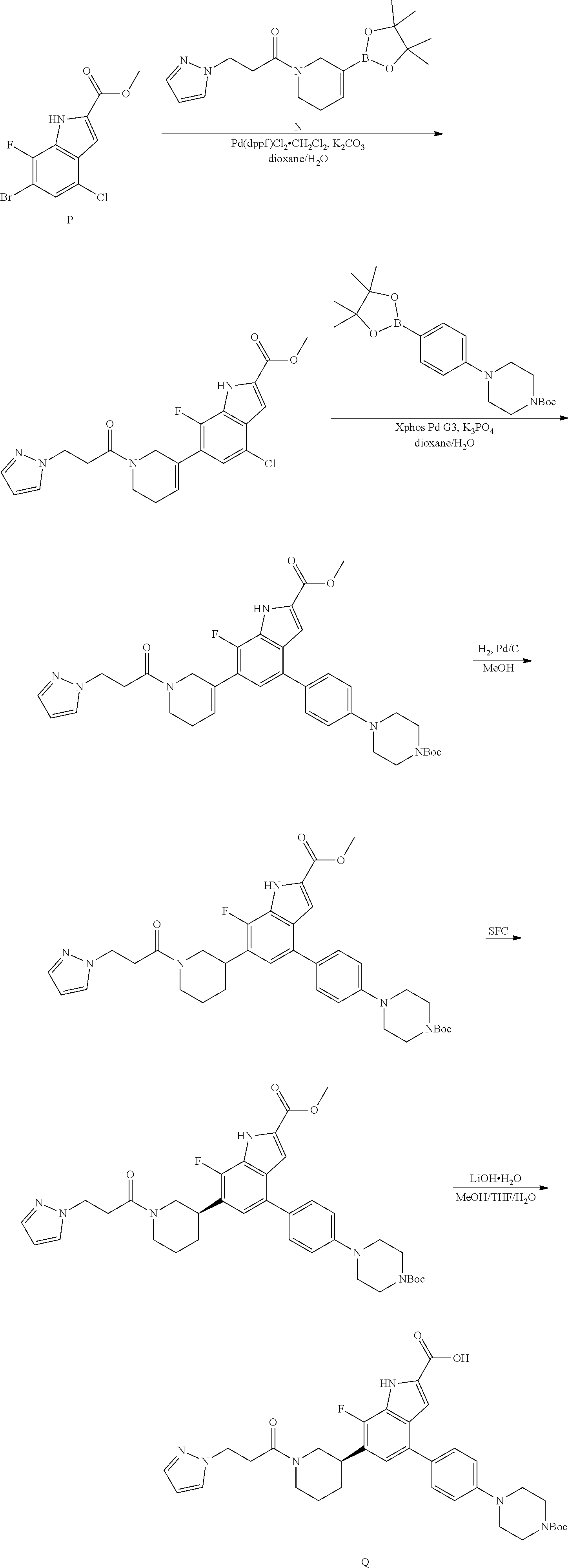

### Step 1—Methyl 6-(1-(3-(1H-pyrazol-1-yl)propanoyl)-1,2,5,6-tetrahydropyridin-3-yl)-4-chloro-7-fluoro-1H-indole-2-carboxylate

To a solution of methyl 6-bromo-4-chloro-7-fluoro-1H-indole-2-carboxylate (2.2 g, 7.18 mmol, Intermediate P), 3-(1H-pyrazol-1-yl)-1-(5-(4,4,5,5-tetramethyl-1,3,2-dioxaborolan-2-yl)-3,6-dihydropyridin-1(2H)-yl)propan-1-one (2.40 g, 7.25 mmol, Intermediate N) in dioxane (30 mL) and H₂O (10 mL) was added and Pd(dppf)Cl₂·CH₂Cl₂ (586 mg, 717 μmol) and K₂CO₃ (2.98 g, 21.5 mmol) under nitrogen atmosphere. Then the reaction was stirred at 60° C. for 2 h under nitrogen atmosphere. On completion, the reaction mixture was diluted with water (10 mL) and extracted with ethyl acetate (3×10 mL). The combined organic layers were dried over anhydrous sodium sulfate, filtered and the filtrate was concentrated in vacuo to give the crude residue. The residue was purified by column chromatography (SiO₂, DCM:EA=10/1 to 0/1) to give the title compound (2.2 g, 71% yield) as a yellow solid. LC-MS (ESI⁺) n z 431.3 (M+H)⁺.

### Step 2—Methyl 6-(1-(3-(1H-pyrazol-1-yl)propanoyl)-1,2,5,6-tetrahydropyridin-3-yl)-4-(4-(4-(tert-butoxycarbonyl)piperazin-1-yl)phenyl)-7-fluoro-1H-indole-2-carboxylate

To a solution of methyl 6-(1-(3-(1H-pyrazol-1-yl)propanoyl)-1,2,5,6-tetrahydropyridin-3-yl)-4-chloro-7-fluoro-1H-indole-2-carboxylate (2.2 g, 5.11 mmol) and tert-butyl 4-(4-(4,4,5,5-tetramethyl-1,3,2-dioxaborolan-2-yl)phenyl)piperazine-1-carboxylate (2.18 g, 5.62 mmol, CAS #470478-90-1) in dioxane (30 mL) and H₂O (10 mL) was added and XPhos Pd G3 (432 mg, 510 μmol) and K₃PO₄ (3.25 g, 15.3 mmol) under nitrogen atmosphere. Then the reaction was stirred at 60° C. for 2 h under nitrogen atmosphere. On completion, the reaction mixture was diluted with water (10 mL) and extracted with ethyl acetate (3×10 mL). The combined organic layers were dried over anhydrous sodium sulfate, filtered and the filtrate was concentrated in vacuo to give the crude residue. The residue was purified by column chromatography (SiO₂, DCM:EA=10/1 to 0/1) to give the title compound (2.8 g, 84% yield) as a yellow solid. LC-MS (ESI⁺) m/z 657.3 (M+H)⁺.

### Step 3—Methyl 6-(1-(3-(1H-pyrazol-1-yl)propanoyl)piperidin-3-yl)-4-(4-(4-(tert-butoxycarbonyl)piperazin-1-yl)phenyl)-7-fluoro-1H-indole-2-carboxylate

To a solution of methyl 6-(1-(3-(1H-pyrazol-1-yl)propanoyl)-1,2,5,6-tetrahydropyridin-3-yl)-4-(4-(4-(tert-butoxycarbonyl)piperazin-1-yl)phenyl)-7-fluoro-1H-indole-2-carboxylate (2.5 g, 3.81 mmol) in MeOH (30 mL) was added Pd/C (2.5 g, 2.35 mmol, 10 wt %) under Ar. The suspension was degassed under vacuum and purged with H₂ several times. The mixture was then stirred under H₂ (15 psi) at 30° C. for 12 h. On completion, the reaction mixture was filtered to give the filtrate, and the reaction mixture was concentrated under reduced pressure to give the title compound (2.6 g) as a white solid. LC-MS (ESI⁺) m/z 659.3 (M+H)⁺.

### Step 4—Methyl (R)-6-(1-(3-(1H-pyrazol-1-yl)propanoyl)piperidin-3-yl)-4-(4-(4-(tert-butoxycarbonyl)piperazin-1-yl)phenyl)-7-fluoro-1H-indole-2-carboxylate

A solution of methyl 6-(1-(3-(1H-pyrazol-1-yl)propanoyl)piperidin-3-yl)-4-(4-(4-(tert-butoxycarbonyl)piperazin-1-yl)phenyl)-7-fluoro-1H-indole-2-carboxylate was separated by SFC (column: REGIS (s,s) WHELK-O1(250 mm×50 mm, 10 um); mobile phase: [CO₂-ACN/MeOH(0.1% NH₃H₂O)]; B %:70%, isocratic elution mode) to give methyl (R)-6-(1-(3-(1H-pyrazol-1-yl)propanoyl)piperidin-3-yl)-4-(4-(4-(tert-butoxycarbonyl)piperazin-1-yl)phenyl)-7-fluoro-1H-indole-2-carboxylate (650 mg, 24% yield) as a white solid. LC-MS (ESI⁺) m/z 659.3 (M+H)⁺ for both isomers. The absolute stereochemistry of the enantiomers was assigned arbitrarily.

### Step 5—(R)-6-(1-(3-(1H-pyrazol-1-yl)propanoyl)piperidin-3-yl)-4-(4-(4-(tert-butoxycarbonyl)piperazin-1-yl)phenyl)-7-fluoro-1H-indole-2-carboxylic acid

To a solution of methyl (R)-6-(1-(3-(1H-pyrazol-1-yl)propanoyl)piperidin-3-yl)-4-(4-(4-(tert-butoxycarbonyl)piperazin-1-yl)phenyl)-7-fluoro-1H-indole-2-carboxylate (600 mg, 910 μmol) in MeOH (1 mL) and THF (4 mL) and H₂O (1 mL) was added LiOH·H₂O (382 mg, 9.11 mmol). The mixture was then stirred at 40° C. for 2 h. On completion, HCl (1 mol) was added to the reaction mixture until the pH 7, then diluted with water (10 mL) and extracted with ethyl acetate (3×10 mL). The combined organic layers were dried over anhydrous sodium sulfate, filtered and the filtrate was concentrated in vacuo to give the title compound (0.8 g) as a yellow solid. LC-MS (ESI⁺) m/z 645.4 (M+H)⁺.

### (R)-1-(3-(2-(azetidine-1-carbonyl)-7-fluoro-4-(4-(piperazin-1-yl)phenyl)-1H-indol-6-yl)piperidin-1-yl)-3-(1H-pyrazol-1-yl)propan-1-one (Intermediate R)

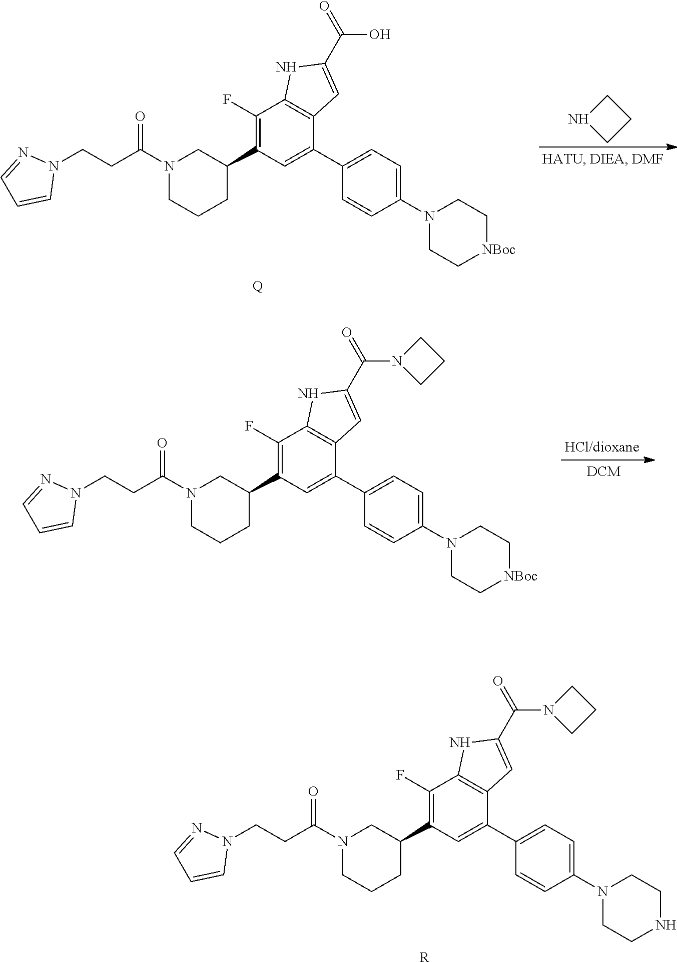

### Step 1—Tert-butyl (R)-4-(4-(6-(1-(3-(1H-pyrazol-1-yl)propanSoyl)piperidin-3-yl)-2-(azctidine-1-carbonyl)-7-fluoro-1H-indol-4-yl)phenyl)piperazine-1-carboxylate

To a solution of (R)-6-(1-(3-(1H-pyrazol-1-yl)propanoyl)piperidin-3-yl)-4-(4-(4-(tert-butoxycarbonyl)piperazin-1-yl)phenyl)-7-fluoro-1H-indole-2-carboxylic acid (135 mg, 209 μmol, Intermediate Q) and azetidine (23.5 mg, 251 μmol, CAS #503-29-7) in DMF (2 mL) was added HATU (95.5 mg, 251 mol) and DIEA (108 mg, 838 μmol). Then the mixture was stirred at 0° C. for 15 min. On completion, the crude product was triturated with H₂O at 20° C. for 1 hr, then the mixture was filtered and the filter cake was triturated with PE at 20° C. for 1 hr. The filter cake was filtered and dried to give the title compound (150 mg, crude) as a yellow solid. LC-MS (ESI⁺) m/z 684.7 (M+H)⁺.

### Step 2—(R)-1-(3-(2-(azetidine-1-carbonyl)-7-fluoro-4-(4-(piperazin-1-yl)phenyl)-1H-indol-6-yl)piperidin-1-yl)-3-(1H-pyrazol-1-yl)propan-1-one

A solution of tert-butyl (R)-4-(4-(6-(1-(3-(1H-pyrazol-1-yl)propanoyl)piperidin-3-yl)-2-(azetidine-1-carbonyl)-7-fluoro-1H-indol-4-yl)phenyl)piperazine-1-carboxylate (150 mg, 219 mol) in TFA (1 mL) and DCM (5 mL) was stirred at 25° C. for 0.5 hrs. On completion, the reaction mixture was concentrated under reduced pressure to give the title compound (150 mg) as a yellow oil. LC-MS (ESI⁺) m/z 584.5 (M+H)⁺.

### 2-(1-(2,6-Dioxopiperidin-3-yl)-3-methyl-2-oxo-2,3-dihydro-1H-benzo[d]imidazol-4-yl)acetaldehyde (Intermediate S)

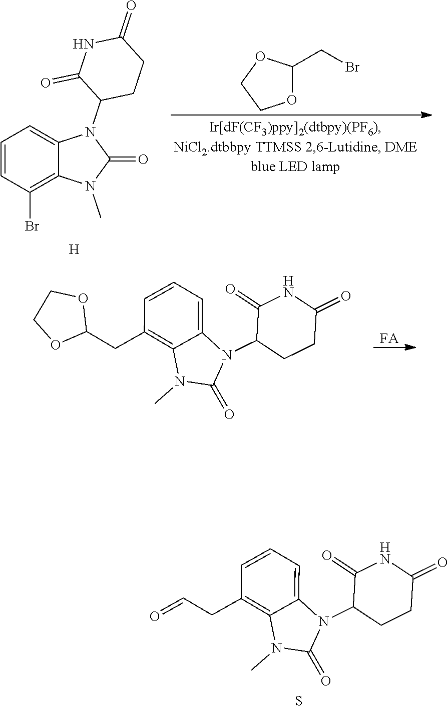

### Step 1 3-(4-((1,3-dioxolan-2-yl)methyl)-3-methyl-2-oxo-2,3-dihydro-1H-benzo[d]imidazol-1-yl)piperidine-2,6-dione

To a 40 mL vial equipped with a stir bar was added 3-(4-bromo-3-methyl-2-oxo-2,3-dihydro-1H-benzo[d]imidazol-1-yl)piperidine-2,6-dione (2 g, 5.91 mmol, Intermediate H), 2-(bromomethyl)-1,3-dioxolane (1.28 g, 7.69 mmol, 788 μL, CAS #4360-63-8), bis[3,5-difluoro-2-[5-(trifluoromethyl)-2-pyridyl]phenyl]iridium(1+) 4-tert-butyl-2-(4-tert-butyl-2-pyridyl)pyridine hexafluorophosphate (133 mg, 118 μmol), 4-tert-butyl-2-(4-tert-butyl-2-pyridyl)pyridine; dichloronickel (70.6 mg, 177 μmol), bis(trimethylsilyl)silyl-trimethyl-silane (1.76 g, 7.10 mmol, 2.19 mL), and 2,6-lutidine (1.27 g, 11.8 mmol, 1.38 mL) in DME (120 mL). The vial was sealed and placed under nitrogen and the reaction was stirred and irradiated with a 4×50 W [455 nm] blue LED lamp (3 cm away), with cooling water to keep the reaction temperature at 25° C. for 14 h. On completion, the reaction mixture was poured into water (150 mL), and then extracted with EtOAc (250 mL×3). The combined organic layers were washed with brine (200 mL×3), dried over Na₂SO₄, filtered and concentrated under reduced pressure to give a residue. The residue was purified by column chromatography (SiO₂, Petroleum ether/Ethyl acetate=0/1) to afford the title compound (870 mg, 40% yield) as a white solid. ¹H NMR (400 MHz, DMSO-d₆) δ=11.09 (s, 1H), 7.08-6.86 (m, 3H), 5.37 (dd, J=5.6, 12.8 Hz, 1H), 5.02 (t, J=4.8 Hz, 1H), 3.94-3.83 (m, 2H), 3.81-3.70 (m, 2H), 3.57 (s, 3H), 3.24 (d, J=4.8 Hz, 2H), 2.95-2.82 (m, 1H), 2.74-2.59 (m, 2H), 2.04-1.96 (m, 1H).

### Step 2—2-(1-(2,6-Dioxopiperidin-3-yl)-3-methyl-2-oxo-2,3-dihydro-1H-benzo[d]imidazol-4-yl)acetaldehyde

To a solution of 3-(4-((1,3-dioxolan-2-yl)methyl)-3-methyl-2-oxo-2,3-dihydro-1H-benzo[d]imidazol-1-yl)piperidine-2,6-dione (200 mg, 579 μmol) in FA (10 mL). The mixture was then stirred at 25° C. for 12 h. On completion, the reaction mixture was concentrated under reduced pressure to afford the title compound (237 mg, FA) as a yellow oil. LC-MS (ESI⁺) m/z 302.3 (M+H)⁺.

### 6-bromo-4-chloro-7-fluoro-N,N-dimethyl-1H-indole-2-carboxamide (Intermediate T)

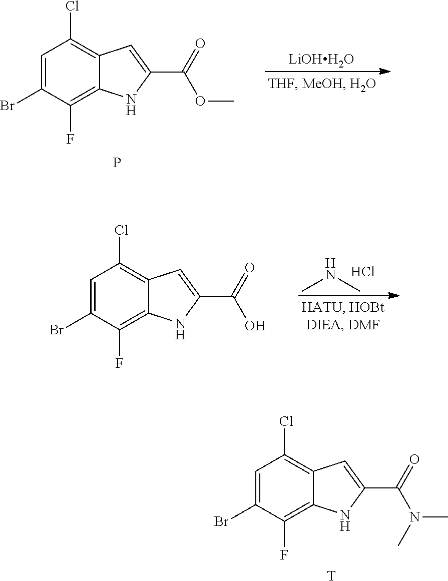

### Step 1—6-Bromo-4-chloro-7-fluoro-1H-indole-2-carboxylic acid

To a solution of methyl 6-bromo-4-chloro-7-fluoro-1H-indole-2-carboxylate (2 g, 6.52 mmol, Intermediate P) in THF (20 mL). H₂O (5 mL), and MeOH (5 mL) was added LiOH·H₂O (821 mg, 19.6 mmol), then the reaction was stirred at 25° C. for 12 hrs. On completion, the reaction mixture was diluted with water (200 mL). Then the reaction mixture was filtered and the filter cake was dried in vacuo to give the title compound (1.8 g) as a yellow solid. LC-MS (ESI+) m/z 291.8 (M−H)⁺.

### Step 2—6-Bromo-4-chloro-7-fluoro-N,N-dimethyl-1H-indole-2-carboxamide

To a solution of 6-bromo-4-chloro-7-fluoro-1H-indole-2-carboxylic acid (500 mg, 1.71 mmol) in DMF (5 mL), was added HATU (779 mg, 2.05 mmol), HOBt (346 mg, 2.56 mmol). DIEA (1.10 g, 8.55 mmol, 1.49 mL), and dimethylamine hydrochloride (209 mg, 2.56 mmol. CAS #506-59-2). Then the reaction was stirred at 25° C. for 2 hrs. On completion, the reaction mixture was diluted with water (20 mL), and the reaction mixture was filtered. The filter cake was dried in vacuo to give the title compound (500 mg) as a yellow solid. LC-MS (ESI⁺) m/z 318.8 (M+H)⁺.

### 2-Methyl-1-(5-(4,4,5,5-tetramethyl-1,3,2-dioxaborolan-2-yl)-3,6-dihydropyridin-1(2H)-yl)propan-1-one (Intermediate U)

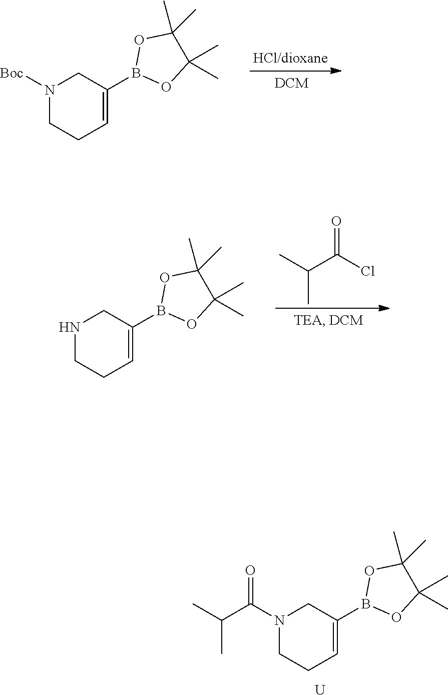

### Step 1—5-(4,4,5,5-Tetramethyl-1,3,2-dioxaborolan-2-yl)-1,2,3,6-tetrahydropyridine

To a solution of tert-butyl 5-(4,4,5,5-tetramethyl-1,3,2-dioxaborolan-2-yl)-3,6-dihydropyridine-1(2H)-carboxylate (18.0 g, 58.2 mmol, CAS #885693-20-9) in DCM (110 mL) was added HCl/dioxane (4.0 M, 90.0 mL), then the mixture was stirred at 25° C. for 1 h. On completion, the mixture was concentrated to give the title compound (28.1 g, 91% yield, HCl) as a yellow solid. LC-MS (ESI⁺) m/z 210.1 (M+H)⁺.

### Step 2—2-Methyl-1-(5-(4,4,5,5-tetramethyl-1,3,2-dioxaborolan-2-yl)-3,6-dihydropyridin-1(2H)-yl)propan-1-one

To a solution of 5-(4,4,5,5-tetramethyl-1,3,2-dioxaborolan-2-yl)-1,2,3,6-tetrahydropyridine (6 g, 28.7 mmol. HCl) in DCM (100 mL) was added TEA (11.6 g, 114 mmol, 15.9 mL). Then was added isobutyryl chloride (6.12 g, 57.4 mmol, 6.01 mL, CAS #79-30-1) was added at 0° C. The mixture was then stirred at 25° C. for 4 h. On completion, the reaction mixture was quenched by addition of NH₄Cl solution (20 mL) at 0° C., and then diluted with H₂O (20 mL) and extracted with DCM (300 mL). The combined organic layers were concentrated under reduced pressure to give a residue. The residue was purified by column chromatography (SiO₂, Petroleum ether/Ethyl acetate=10/1 to 3/1) to give the title compound (7 g, 83% yield) as a white gum. LC-MS (ESI⁺) m/z 280.3 (M+H)⁺.

### 4-Chloro-7-fluoro-6-(1-isobutyryl-1,2,5,6-tetrahydropyridin-3-yl)-N,N-dimethyl-1H-indole-2-carboxamide (Intermediate V)

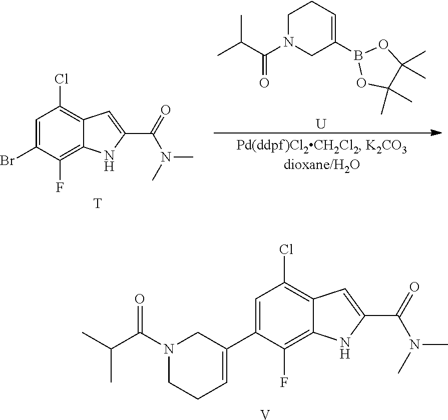

To a solution of 6-bromo-4-chloro-7-fluoro-N,N-dimethyl-1H-indole-2-carboxamide (450 mg, 1.41 mmol, Intermediate T), 2-methyl-1-(5-(4,4,5,5-tetramethyl-1,3,2-dioxaborolan-2-yl)-3,6-dihydropyridin-1(2H)-yl)propan-1-one (589 mg, 2.11 mmol, Intermediate U) in dioxane (5 mL) and H₂O (1.5 mL) was added K₂CO₃ (584 mg, 4.22 mmol) and Pd(dppf)Cl₂·CH₂Cl₂ (115 mg, 140 μmol). Then the reaction was stirred at 80° C. for 2 hrs under nitrogen atmosphere. On completion, the reaction mixture was diluted with water (20 mL) and extracted with ethyl acetate (4×30 mL). The combined organic layers were washed with brine (20 mL) and dried over anhydrous sodium sulfate, filtered and the filtrate was concentrated in vacuo to give the crude residue. The residue was purified by column chromatography (SiO₂, Petroleum ether:Ethyl acetate=10/1 to 1/1) to give the title compound (500 mg, 89% yield) as a red oil. LC-MS (ESI⁺) m/z 390.1 (M−H)⁻.

### Tert-butyl 4-[3-(difluoromethoxy)-4-(4,4,5,5-tetramethyl-1,3,2-dioxaborolan-2-yl)phenyl]piperazine-1-carboxylate (Intermediate W)

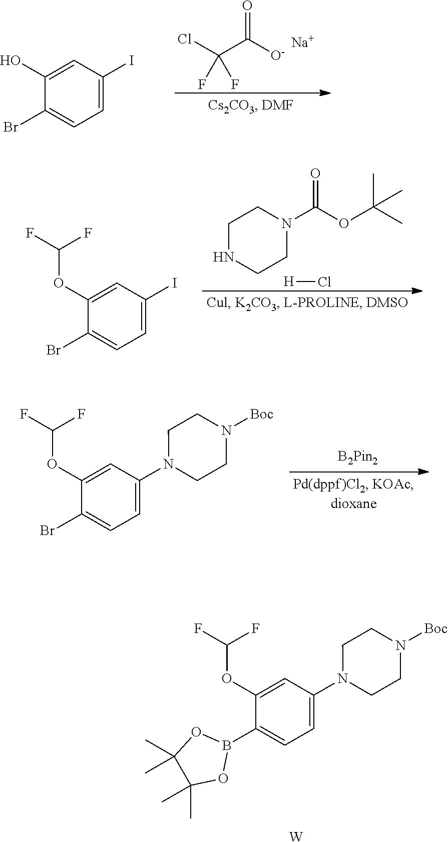

### Step 1—1-Bromo-2-(difluoromethoxy)-4-iodo-benzene

To a solution of 2-bromo-5-iodo-phenol (6.00 g, 20.0 mmol, CAS #932372-99-1) and sodium 2-chloro-2,2-difluoroacetate (7.65 g, 50.1 mmol. CAS #1895-39-2) in DMF (270 mL) and H₂O (30.0 mL) was added Cs₂CO₃ (13.0 g, 40.1 mmol). The mixture was then stirred at 100° C. for 12 hrs. On completion, the mixture was diluted with water (1000 mL) and extracted with ethyl acetate (500 mL×2). The organic layer was washed with aq. NaHCO₃, dried over sodium sulfate, filtered and concentrated under reduced pressure to give the title compound (7.00 g, 90% yield) as a white solid. ¹H NMR (400 MHz, CDCl₃) δ=7.55 (d, J=0.8 Hz, 1H), 7.44-7.46 (m, 1H), 7.33 (d, J=8.4 Hz, 1H), 6.53-6.56 (m, 1H).

### Step 2—Tert-butyl 4-[4-bromo-3-(difluoromethoxy)phenyl]piperazine-1-carboxylate

To a solution of 1-bromo-2-(difluoromethoxy)-4-iodo-benzene (8.40 g, 24.0 mmol) and tert-butyl piperazine-1-carboxylate hydrochloride (10.7 g, 48.1 mmol, CAS #76535-74-5) in DMSO (80.0 mL) was added CuI (917 mg, 4.81 mmol), K₂CO₃ (9.98 g, 72.2 mmol) and L-proline (554 mg, 4.81 mmol). The mixture was then stirred at 80° C. for 12 hrs. On completion, the mixture was diluted with water (15 mL) and extracted with ethyl acetate (15 mL×2). The combined organic layer was dried over sodium sulfate, filtered and concentrated under reduced pressure. The residue was purified by column chromatography (SiO₂, petroleum ether:ethyl acetate=10/1) to give the title compound (13.6 g) as a white solid. LC-MS (ESI⁺) m/z 407.1 (M+H)⁺.

### Step 3—Tert-butyl 4-[3-(difluoromethoxy)-4-(4,4,5,5-tetramethyl-1,3,2-dioxaborolan-2-yl)phenyl]piperazine-1-carboxylate

To a solution of tert-butyl 4-[4-bromo-3-(difluoromethoxy)phenyl]piperazine-1-carboxylate (4.90 g, 12.0 mmol) and 4,4,5,5-tetramethyl-2-(4,4,5,5-tetramethyl-1,3,2-dioxaborolan-2-yl)-1,3,2-dioxaborolane (15.2 g, 60.1 mmol) in dioxane (50.0 mL) was added KOAc (3.54 g, 36.1 mmol) and Pd(dppf)Cl₂ (880 mg, 1.20 mmol). The mixture was then stirred at 90° C. for 8 hrs. Additional 4,4,5,5-tetramethyl-2-(4,4,5,5-tetramethyl-1,3,2-dioxaborolan-2-yl)-1,3,2-dioxaborolane (9.17 g, 36.1 mmol) and Pd(dppf)Cl₂ (880 mg, 1.20 mmol) was added and the mixture was stirred at 90° C. for 4 hrs. On completion, the mixture was diluted with water (100 mL) and extracted with ethyl acetate (100 mL×2). The combined organic layer was dried over sodium sulfate, filtered and concentrated under reduced pressure. The residue was purified by column chromatography (SiO₂, petroleum ether:ethyl acetate=20/1) to give the title compound (3.50 g, 56% yield) as a yellow oil. ¹H NMR (400 MHz, CDCl₃) δ=7.66 (d, J=8.4 Hz, 1H), 6.79-6.70 (m, 1H), 6.63 (d, J=2.0 Hz, 1H), 6.54-6.28 (m, 1H), 3.64-3.52 (m, 4H), 3.29-3.12 (m, 4H), 1.49 (s, 9H), 1.33 (s, 11H).

### 4-(2-(Difluoromethoxy)-4-(piperazin-1-yl)phenyl)-7-fluoro-6-(1-isobutyryl-1,2,5,6-tetrahydropyridin-3-yl)-N,N-dimethyl-1H-indole-2-carboxamide (Intermediate X)

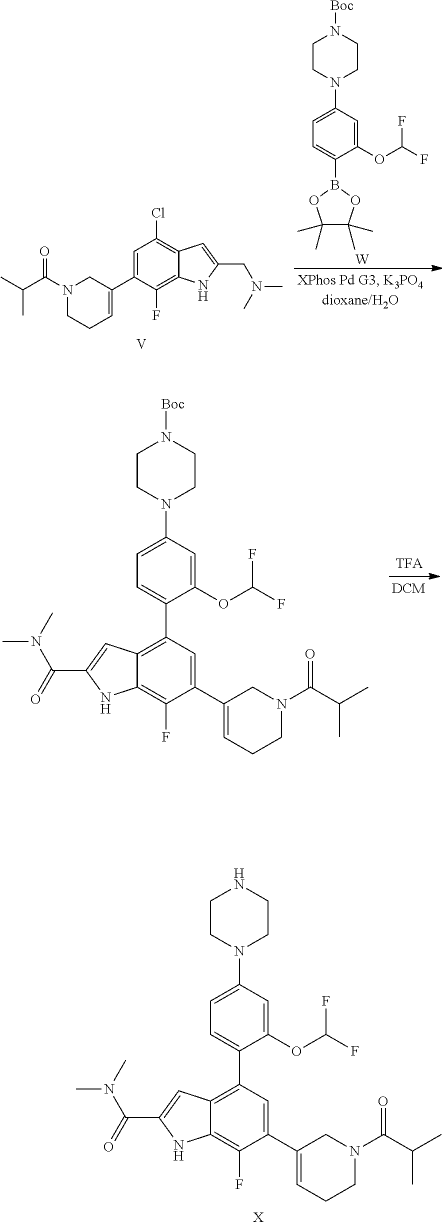

### Step 1—Tert-butyl 4-(3-(difluoromethoxy)-4-(2-(dimethylcarbamoyl)-7-fluoro-6-(1-isobutyryl-1,2,5,6-tetrahydropyridin-3-yl)-1H-indol-4-yl)phenyl)piperazine-1-carboxylate

To a solution of 4-chloro-7-fluoro-6-(1-isobutyryl-1,2,5,6-tetrahydropyridin-3-yl)-N,N-dimethyl-1H-indole-2-carboxamide (200 mg, 510 μmol, Intermediate V), tert-butyl 4-(3-(difluoromethoxy)-4-(4,4,5,5-tetramethyl-1,3,2-dioxaborolan-2-yl)phenyl)piperazine-1-carboxylate (278 mg, 612 μmol, Intermediate W) in dioxane (2 mL) and H₂O (0.6 mL) was added K₃PO₄ (325 mg, 1.53 mmol) and XPhos Pd G3 (43.2 mg, 51.0 μmol). Then the reaction was stirred at 80° C. for 2 hrs under nitrogen atmosphere. On completion, the reaction mixture was diluted with water (20 mL) and extracted with ethyl acetate (4×30 mL). The combined organic layers were washed with brine (20 mL), dried over anhydrous sodium sulfate, filtered and the filtrate was concentrated in vacuo to give the crude residue. The residue was purified by column chromatography (SiO₂, Petroleum ether:Ethyl acetate=10/1 to 1/1) to give the title compound (300 mg, 82% yield) as a yellow solid. LC-MS (ESI⁺) m/z 684.1 (M+H)f.

### Step 2—4-(2-(difluoromethoxy)-4-(piperazin-1-yl)phenyl)-7-fluoro-6-(1-isobutyryl-1,2,5,6-tetrahydropyridin-3-yl)-N,N-dimethyl-1H-indole-2-carboxamide

A mixture of tert-butyl 4-(3-(difluoromethoxy)-4-(2-(dimethylcarbamoyl)-7-fluoro-6-(1-isobutyryl-1,2,5,6-tetrahydropyridin-3-yl)-1H-indol-4-yl)phenyl)piperazine-1-carboxylate (280 mg, 409 gmol) in DCM (3 mL) and TFA (716 mg, 6.28 mmol, 466 μL) was stirred at 25° C. for 2 hrs. On completion, the reaction mixture was filtered and the filtrate was concentrated in vacuo to give the title compound (250 mg, TFA) as yellow solid. LC-MS (ESI⁺) m/z 584.2 (M+H)⁺.

### Tert-butyl 4-(4-(4,4,5,5-tetramethyl-1,3,2-dioxaborolan-2-yl)-3-(trifluoromethoxy)phenyl) piperazine-1-carboxylate (Intermediate Y)

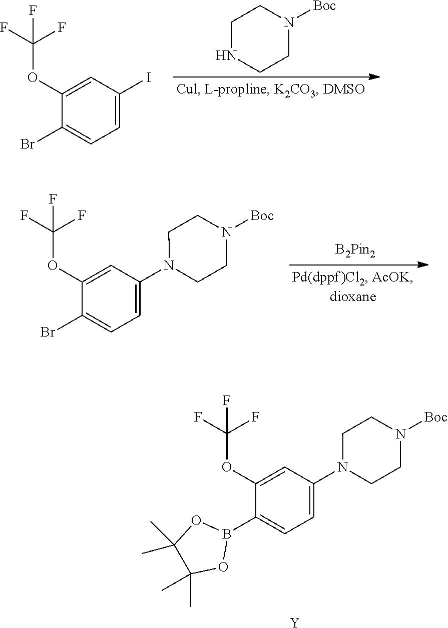

### Step 1—Tert-butyl 4-(4-bromo-3-(trifluoromethoxy)phenyl)piperazine-1-carboxylate

To a solution of 1-bromo-4-iodo-2-(trifluoromethoxy)benzene (4 g, 10.9 mmol, CAS #1187984-18-4), tert-butyl piperazine-1-carboxylate (4.86 g, 21.8 mmol, CAS #57260-71-6) in DMSO (50 mL) was added K₂CO₃ (4.52 g, 32.7 mmol), L-proline (251 mg, 2.18 mmol) and CuI (415 mg, 2.18 mmol). Then the reaction was stirred at 80° C. for 12 hrs under nitrogen atmosphere. On completion, the reaction mixture was diluted with water (50 mL) and dichloromethane (3×50 mL). The combined organic layers were washed with brine (50 mL) and dried over anhydrous sodium sulfate, filtered and the filtrate was concentrated in vacuo to give the crude residue. The residue was purified by column chromatography (SiO₂, Petroleum ether:Ethyl acetate=10/1 to 5/1) to give the title compound (3.3 g, 70% yield) as a yellow solid. LC-MS (ESI⁺) m/z 424.9 (M+H)⁺.

### Step 2—Tert-butyl 4-(4-(4,4,5,5-tetramethyl-1,3,2-dioxaborolan-2-yl)-3-(trifluoromethoxy)phenyl)piperazine-1-carboxylate

To a solution of tert-butyl 4-(4-bromo-3-(trifluoromethoxy)phenyl)piperazine-1-carboxylate (3.00 g, 7.05 mmol) and B₂Pin₂ (8.96 g, 35.3 mmol) in dioxane (30.0 mL) was added KOAc (2.08 g, 21.7 mmol) and Pd(dppf)Cl₂ (516 mg, 706 gmol). Then the mixture was stirred at 80° C. for 2 hrs under N₂ atmosphere. On completion, the reaction mixture was diluted with water (100 mL) and extracted with EA (30.0 mL×3). The combined organic layers were washed with brine (100 mL×2), dried over Na₂SO₄, filtered and concentrated under reduced pressure to give a residue. The residue was purified by flash silica gel chromatography (ISCO®; 12 g SepaFlash® Silica Flash Column, Eluent of 0˜5% Ethyl acetate/Petroleum ethergradient @20 mL/min) to give the title compound (2 g, 59% yield) as a white solid. LC-MS (ESI⁺) m/z 473.2 (M+H)⁺.

### 6-(1-Acetyl-1,2,5,6-tetrahydropyridin-3-yl)-4-chloro-7-fluoro-N,N-dimethyl-1H-indole-2-carboxamide (Intermediate Z)

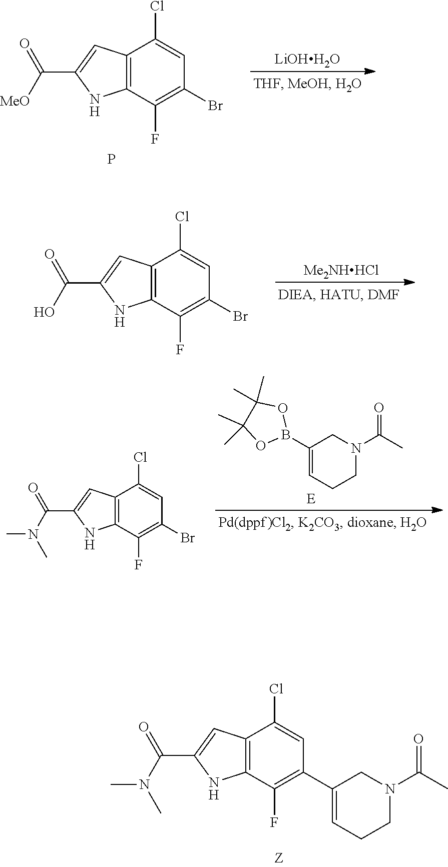

### Step 1—6-Bromo-4-chloro-7-fluoro-1H-indole-2-carboxylic acid

To a solution of methyl 6-bromo-4-chloro-7-fluoro-1H-indole-2-carboxylate (10 g, 32.6 mmol. Intermediate P) in THF (80 mL). MeOH (20 mL) and H₂O (20 mL) was added LiOH·H₂O (6.85 g, 163 mmol). The reaction was then stirred at 50° C. for 12 hrs. On completion, the reaction mixture was added HCl (1N) until the pH=7. Then the mixture was concentrated in vacuo and lyophilized to give the title compound (10.9 g) as a white solid.

### Step 2—6-Bromo-4-chloro-7-fluoro-N,N-dimethyl-1H-indole-2-carboxamide

To a solution of 6-bromo-4-chloro-7-fluoro-1H-indole-2-carboxylic acid (8.9 g, 30.4 mmo) in DMF (100 mL) was added N-methylmethanamine (4.96 g, 60.9 mmol, 5.58 mL, HCl), DIEA (11.8 g, 91.3 mmol, 15.9 mL) and HATU (12.7 g, 33.5 mmol). The reaction was then stirred at 25° C. for 1 hr. On completion, the reaction mixture was quenched with water (300 mL) and mixture was filtered and the filter cake was concentrated in vacuo to give the title compound (7.76 g) as a white solid. LC-MS (ESI+) m/z 319.0. (M+H)⁺.

### Step 3—6-(1-Acetyl-1,2,5,6-tetrahydropyridin-3-yl)-4-chloro-7-fluoro-N,N-dimethyl-1H-indole-2-carboxamide

To a solution of 6-bromo-4-chloro-7-fluoro-N,N-dimethyl-1H-indole-2-carboxamide (6.74 g, 21.1 mmol) in dioxane (80 mL) and H₂O (20 mL) was added 1-(5-(4,4,5,5-tetramethyl-1,3,2-dioxaborolan-2-yl)-3,6-dihydropyridin-1(2H)-yl)ethan-1-one (5.3 g, 21.1 mmol, Intermediate E), K₂CO₃ (8.75 g, 63.3 mmol) and Pd(dppf)Cl₂ (1.54 g, 2.11 mmol). The reaction was then stirred at 80° C. for 2 hrs under N₂ atmosphere. On completion, the reaction mixture was quenched with water (100 mL) and extracted with ethyl acetate (3×200 mL). The combined organic layers were washed with brine (200 mL) and dried over anhydrous sodium sulfate, filtered and the filtrate was concentrated in vacuo to give the title compound (8 g) as a brown oil. LC-MS (ESI+) m/z 364.2. (M+H)⁺.

### 6-(1-Acetyl-1,2,5,6-tetrahydropyridin-3-yl)-7-fluoro-N,N-dimethyl-4-(4-(piperazin-1-yl)-2-(trifluoromethoxy)phenyl)-1H-indole-2-carboxamide (Intermediate AA)

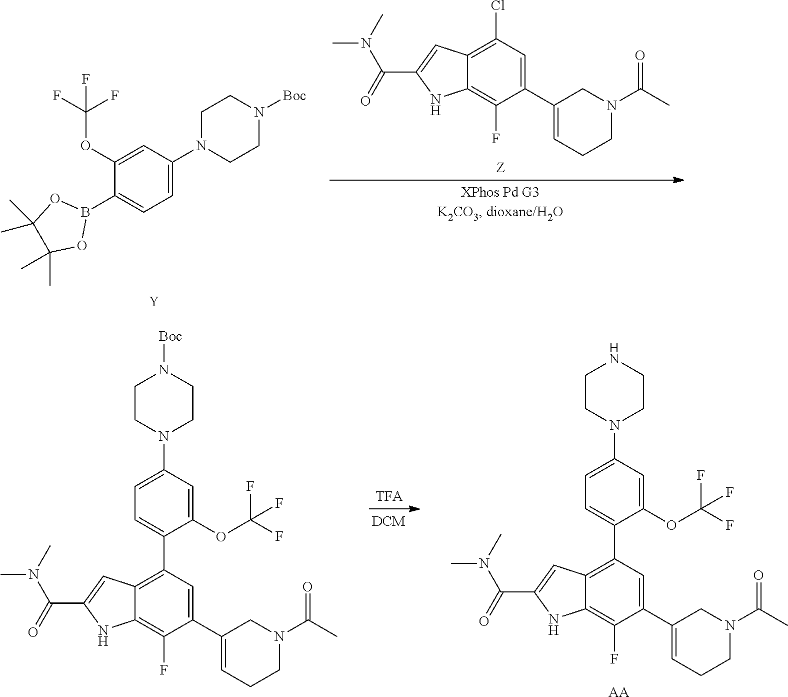

### Step 1—Tert-butyl 4-(4-(6-(1-acetyl-1,2,5,6-tetrahydropyridin-3-yl)-2-(dimethylcarbamoyl)-7-fluoro-1H-indol-4-yl)-3-(trifluoromethoxy)phenyl)piperazine-1-carboxylate

To a solution of 6-(I-acetyl-1,2,5,6-tetrahydropyridin-3-yl)-4-chloro-7-fluoro-NN-dimethyl-1H -indole-2-carboxamide (770 mg, 2.12 mmol, Intermediate Z) and tert-butyl 4-(4-(4,4,5,5-tetramethyl-1,3,2-dioxaborolan-2-yl)-3-(trifluoromethoxy)phenyl)piperazine-1-carboxylate (1.00 g, 2.12 mmol, Intermediate Y) in dioxane (10.0 mL) and H₂O (1.00 mL) was added XPhos Pd G3 (179 mg, 211 mol) and K₃PO₄ (1.35 g, 6.35 mmol). Then the mixture was stirred at 80° C. for 2 hrs under N₂ atmosphere. On completion, the reaction mixture was diluted with water (50 mL) and extracted with EA (10 mL×3). The combined organic layers were washed with brine (30 mL×2), dried over Na₂SO₄, filtered and concentrated under reduced pressure to give a residue. The residue was purified by flash silica gel chromatography (ISCO®; 12 g SepaFlash® Silica Flash Column, Eluent of 20˜80% Ethyl acetate/Petroleum ethergradient @30 mL/min) to give the title compound (1.00 g, 66% yield) as a yellow solid. LC-MS (ESI⁺) m/z 674.5 (M+H)⁺.

### Step 2—6-(1-Acetyl-1,2,5,6-tetrahydropyridin-3-yl)-7-fluoro-N,N-dimethyl-4-(4-(piperazin-1-yl)-2-(trifluoromethoxy)phenyl)-1H-indole-2-carboxamide

To a solution of tert-butyl 4-(4-(6-(I-acetyl-1,2,5,6-tetrahydropyridin-3-yl)-2-(dimethylcarbamoyl)-7-fluoro-1H-indol-4-yl)-3-(trifluoromethoxy)phenyl)piperazine-1-carboxylate (1.00 g, 1.48 mmol) in DCM (10.0 mL) was added TFA (3.07 g, 26.9 mmol), then the mixture was stirred at 20° C. for 1 hr. On completion, the reaction mixture was filtered and concentrated under reduced pressure to give the title compound (2.16 g) as a black oil. LC-MS (ESI⁺) m/z 574.3 (M+H)⁺.

### 2-(1-(2,6-Dioxopiperidin-3-yl)-3-methyl-1H-indazol-4-yl)acetaldehyde (Intermediate AB)

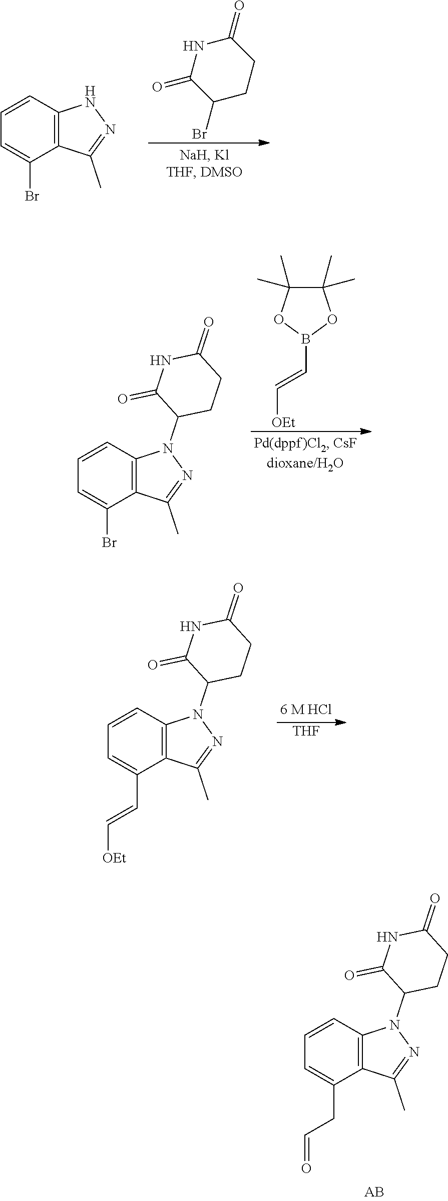

### Step 1—3-(4-Bromo-3-methyl-1H-indazol-1-yl)piperidine-2,6-dione

To a solution of 4-bromo-3-methyl-1H-indazole (2 g, 9.48 mmol. CAS #1159511-73-5) in THF (25 mL) and DMSO (25 mL) was added NaH (1.14 g, 28.4 mmol, 60% dispersion in mineral oil) at 0° C. The mixture was then stirred at 0° C. for 2 hrs. Next, 3-bromopiperidine-2,6-dione (2.73 g, 14.2 mmol, CAS #62595-74-8) and KI (1.26 g, 7.58 mmol) was added and the reaction was stirred at 25° C. for 14 hrs. On completion, the reaction mixture was quenched with FA (5 mL) and extracted with ethyl acetate (3×20 mL). The combined organic layers were washed with brine (50 mL) and dried over anhydrous sodium sulfate, filtered and the filtrate was concentrated in vacuo to give the crude residue. The residue was purified by column chromatography (SiO₂, Petroleum ether:Ethyl acetate=10/1 to 2/1) to give the title compound (2.6 g, 76% yield) as a brown solid. LC-MS (ESI⁺) m/z 323.0. (M+H)⁺.

### Step 2—(E)-3-(4-(2-ethoxyvinyl)-3-methyl-1H-indazol-1-yl)piperidine-2,6-dione

To a solution of (E)-2-(2-ethoxyvinyl)-4,4,5,5-tetramethyl-1,3,2-dioxaborolane (541 mg, 2.73 mmol, CAS #1201905-61-4) in dioxane (10 mL) and H₂O (2 mL) was added 3-(4-bromo-3-methyl-1H-indazol-1-yl)piperidine-2,6-dione (800 mg, 2.48 mmol), CsF (754 mg, 4.97 mmol, 183 pL) and Pd(dppf)Cl₂CH₂Cl₂ (202 mg, 248 μmol). The reaction was then stirred at 90° C. for 2 hrs. On completion, the reaction mixture was concentrated in vacuo to give the crude residue. The residue was purified by column chromatography (SiO₂, Petroleum ether:Ethyl acetate=10/1 to 2/1) to give the title compound (600 mg, 75% yield) as a white solid. LC-MS (ESI⁺) m/z 314.2. (M+H)⁺.

### Step 3—2-(1-(2,6-Dioxopiperidin-3-yl)-3-methyl-1H-indazol-4-yl)acetaldehyde

To a solution of (E)-3-(4-(2-ethoxyvinyl)-3-methyl-1H-indazol-1-yl)piperidine-2,6-dione (800 mg, 2.55 mmol) in THF (1 mL) was added HCl (3 M, 4.00 mL). The mixture was then stirred at 25° C. for 1 hr. On completion, the reaction mixture was used into the next step without further work-up as a gray liquid. LC-MS (ESI⁺) m/z 286.1 (M+H)⁺.

### 4-(4-Chloro-3-methylphenyl)piperidine (Intermediate AC)

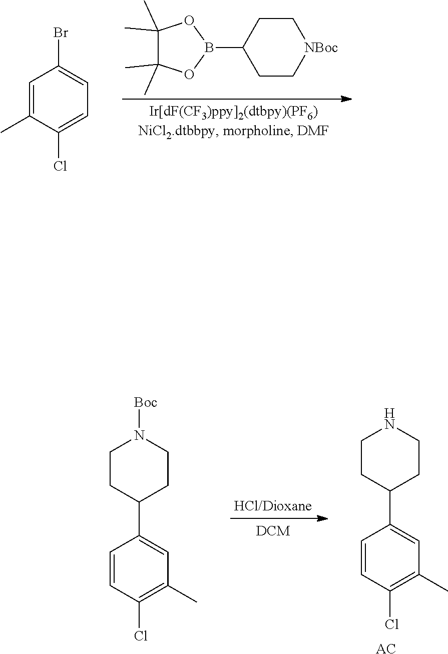

### Step 1—Tert-butyl 4-(4-chloro-3-methylphenyl)piperidine-1-carboxylate

A mixture of 4-bromo-1-chloro-2-methyl-benzene (25.0 g, 122 mmol. CAS #54932-72-8), tert-butyl 4-(4,4,5,5-tetramethyl-1,3,2-dioxaborolan-2-yl)piperidine-1-carboxylate (75.7 g, 243 mmol, CAS #1048970-17-7), bis[3,5-difluoro-2-[5-(trifluoromethyl)-2-pyridyl]phenyl]iridium(1+) 4-tert-butyl-2-(4-tert-butyl-2-pyridyl)pyridine hexafluorophosphate (1.36 g, 1.22 mmol), 4-tert-butyl-2-(4-tert-butyl-2-pyridyl)pyridine dichloronickel (726 mg, 1.83 mmol) and morpholine (15.9 g, 183 mmol) in anhydrous DMF (150 mL) was degassed and purged with nitrogen for three times. Then the reaction was stirred at 25° C. for 12 hrs under nitrogen atmosphere and blue LED. On completion, the reaction mixture was diluted by EA (150 mL) and washed with water (3×50 mL). The combined organic layers were washed with brine (50 mL) and dried over anhydrous sodium sulfate, filtered and the filtrate was concentrated in vacuo to give the crude residue. The crude residue was purified by reverse phase HPLC (0.1% FA condition) to give the title compound (19.0 g, 45% yield) as a yellow oil. LC-MS (ESI⁺) m/z 254.0 (M-55)⁺.

### Step 2—4-(4-Chloro-3-methylphenyl)piperidine

To a solution of tert-butyl 4-(4-chloro-3-methylphenyl)piperidine-1-carboxylate (4.7 g, 15 mmol) in DCM (30 mL) was added HCl/dioxane (4 M, 14 mL). The mixture was stirred at 25° C. for 1 hr. On completion, the mixture was concentrated under reduced pressure. The residue was purified by reverse phasereverse phase HPLC (0.8 g/L ammonium bicarbonate) to give the title compound (3.5 g, 90% yield) as a purple solid. LC-MS (ESI⁺) m/z 210.1 (M+H)⁺: ¹H NMR (400 MHz, DMSO-d6) δ=7.36 (d, J=8.0 Hz, 1H), 7.22 (s, 1H), 7.09 (dd, J=2.0, 8.4 Hz, 1H), 3.27 (d, J=11.2 Hz, 2H), 2.94-2.83 (m, 2H), 2.76 (s, 1H), 2.33 (s, 3H), 1.91-1.71 (m, 4H).

### 3-(4-(4-(4-Chloro-3-methylphenyl)piperidin-1-yl)-3-methyl-2-oxo-2,3-dihydro-1H-benzo[d]imidazol-1-yl)piperidine-2,6-dione (Intermediate AD)

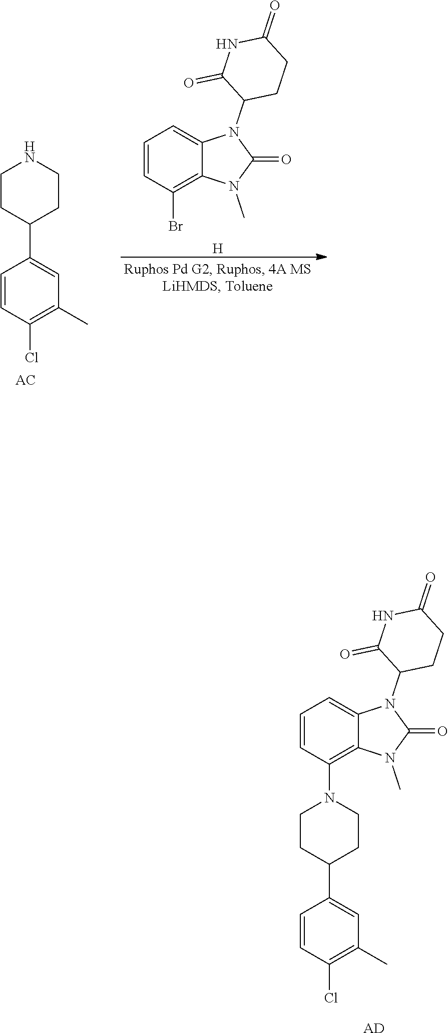

### Step 1—3-(4-(4-(4-Chloro-3-methylphenyl)piperidin-1-yl)-3-methyl-2-oxo-2,3-dihydro-1H-benzo[d]imidazol-1-yl)piperidine-2,6-dione

To a mixture of 4-(4-chloro-3-methylphenyl)piperidine (1.86 g, 8.87 mmol, Intermediate AC), 3-(4-bromo-3-methyl-2-oxo-2,3-dihydro-1H-benzo[d]imidazol-1-yl)piperidine-2,6-dione (2.5 g, 7.39 mmol, Intermediate H) in toluene (100 mL) at 0° C. was added LiHMDS (1 M, 55.5 mL). Then RuPhos (517 mg, 1.11 mmol), [2-(2-aminophenyl)phenyl]-chloro-palladium; dicyclohexyl-[2-(2,6-diisopropoxyphenyl)phenyl]phosphane (574 mg, 739 mol), and 4A MS (4 g) was added and the mixture was degassed and purged with N₂ 3 times. Then the mixture was stirred at 100° C. for 2 hrs under N₂ atmosphere. On completion, the mixture was concentrated under reduced pressure and purified by reverse phasereverse phase HPLC (0.8 g/L ammonium bicarbonate) to give the title compound (750 mg, 22% yield) as a yellow solid. LC-MS (ESI⁺) m/z 467.2 (M+H)⁺.

### 6-(1-Acetyl-1,2,5,6-tetrahydropyridin-3-yl)-7-fluoro-N,N-dimethyl-4-(4,4,5,5-tetramethyl-1,3,2-dioxaborolan-2-yl)-1H-indole-2-carboxamide (Intermediate AE)

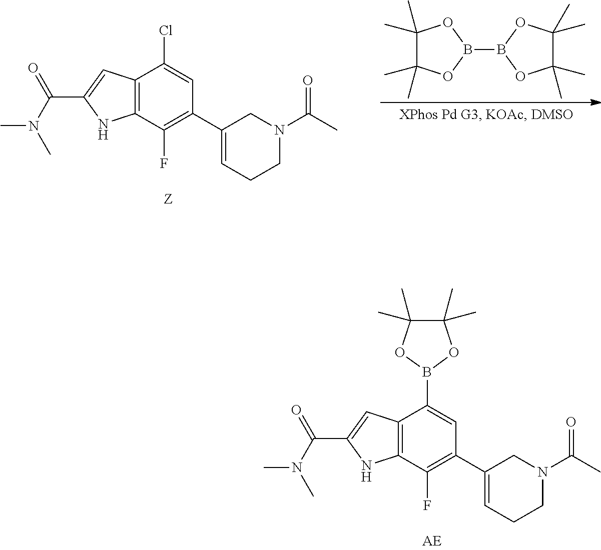

To a solution of 6-(1-acetyl-1,2,5,6-tetrahydropyridin-3-yl)-4-chloro-7-fluoro-N,N-dimethyl-1H-indole-2-carboxamide (8 g, 22.0 mmol, Intermediate Z) in DMSO (100 mL) was added 4,4,5,5-tetramethyl-2-(4,4,5,5-tetramethyl-1,3,2-dioxaborolan-2-yl)-1,3,2-dioxaborolane (13.9 g, 55.0 mmol), KOAc (6.47 g, 65.9 mmol) and XPhos Pd G3 (1.86 g, 2.20 mmol). The reaction was then stirred at 100° C. for 3 hrs. On completion, the reaction mixture was quenched with water (100 mL) and extracted with ethyl acetate (3×200 mL). The combined organic layers were washed with brine (200 mL) and dried over anhydrous sodium sulfate, filtered and the filtrate was concentrated in vacuo to give the crude residue. The residue was purified by column chromatography (SiO₂, Petroleum ether:Ethyl acetate=5/1 to 0/1) to give the title compound (3.5 g) as a yellow solid. LCMS (ESI⁺) m/z 456.1. (M+H)⁺.

### 7-Bromo-3-iodo-1-methyl-1H-indazole (Intermediate AF)

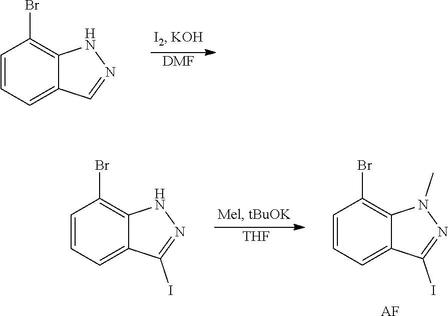

### Step 1—7-Bromo-3-iodo-1H-indazole

To a solution of 7-bromo-1H-indazole (40.0 g, 203 mmol, CAS #53857-58-2) and I₂ (77.3 g, 305 mmol) in DMF (1.1 L) was added KOH (17.1 g, 305 mmol) at 0° C. The mixture was then stirred at 25° C. for 16 hrs. On completion, quenched with water (1 L), followed by saturated sodium sulfite aqueous solution (0.5 L). The reaction mixture was then filtered and the filter cake was dissolved with ethyl acetate (1 L). The organic solution was washed with brine (500 mL×2), dried over anhydrous sodium sulfate, filtered and concentrated under reduced pressure to give the title compound (66 g) as a white solid. LC-MS (ESI⁺) m/z 322.6 (M+H)⁺.

### Step 2—7-Bromo-3-iodo-1-methyl-1H-indazole

To a solution of 7-bromo-3-iodo-1H-indazole (56.0 g, 173 mmol) in THF (1000 mL) was added tBuOK (1 M, 347 mL) dropwise at 0° C. with stirring for 1 hr. Then a solution of iodomethane (44.3 g, 312 mmol) in THE (200 mL) was added dropwise at 0° C. After addition, the reaction mixture was stirred at 25° C. for 14 hrs. On completion, the reaction mixture was filtered and the filter was concentrated under reduced pressure to give a residue. The residue was purified by column chromatography (SiO₂, Petroleum ether/Ethyl acetate=20/1 to 5/1) to give the title compound (33.3 g, 57% yield) as a white solid. LC-MS (ESI⁺) m/z 338.7 (M+H)⁺.

### 3-(2,6-Bis(benzyloxy)pyridin-3-yl)-7-bromo-1-methyl-1H-indazole (Intermediate AG)

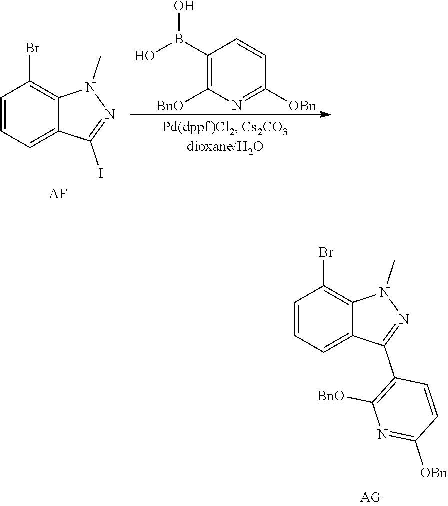 [SMILES: Cn1nc(I)c2cccc(Br)c21 ; OB(O)c1ccc(OCc2ccccc2)nc1OCc1ccccc1 ; "AF" ; Cn1nc(-c2ccc(OCc3ccccc3)nc2OCc2ccccc2)c2cccc(Br)c21]

To a solution of 7-bromo-3-iodo-1-methyl-indazole (16.5 g, 49.0 mmol Intermediate AF) and (2,6-dibenzyloxy-3-pyridyl)boronic acid (16.4 g, 49.0 mmol, CAS #2096339-92-1) in dioxane (660 mL) and H₂O (160 mL) was added Pd(dppf)Cl₂ (3.58 g, 4.90 mmol) and Cs₂CO₃ (47.9 g, 147 mmol). The mixture was the stirred at 100° C. for 2 hrs. On completion, the reaction mixture was quenched with H₂O (2 L) at 25° C., and then diluted with ethyl acetate (1 L) and extracted with ethyl acetate (2 L×2). The combined organic layers were dried over Na₂SO₄, filtered and concentrated under reduced pressure to give a residue. The residue was purified by column chromatography (SiO₂. Petroleum ether/Ethyl acetate=10/1 to 1/1) to give the title compound (23.0 g, 45% yield) as a yellow oil. LC-MS (ESI⁺) m/z 500.0 (M+H)⁺.

### 3-(1-Methyl-7-(4-(3-methyl-4-(4,4,5,5-tetramethyl-1,3,2-dioxaborolan-2-yl)phenyl)piperidin-1-yl)-1H-indazol-3-yl)piperidine-2,6-dione (Intermediate AH)

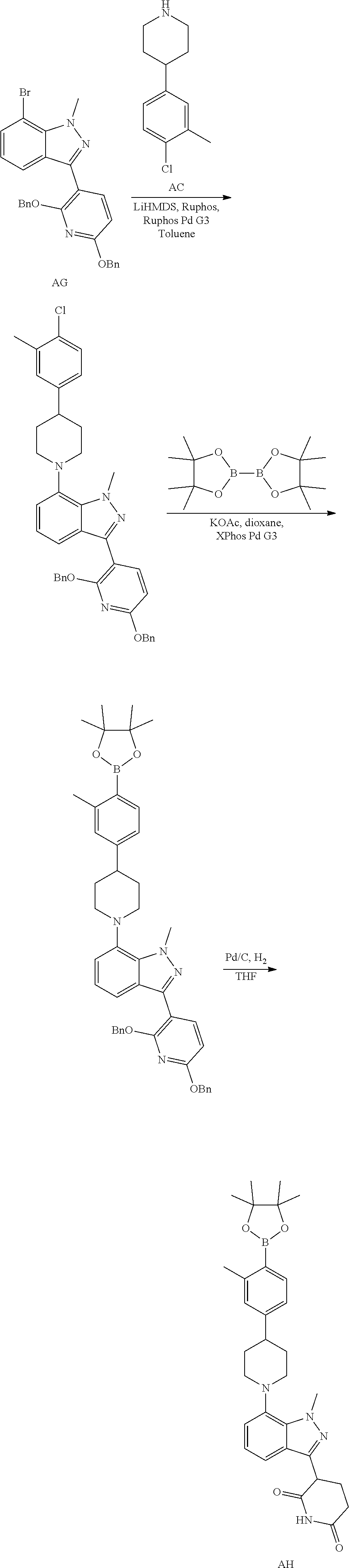

### Step 1—3-(2,6-Bis(benzyloxy)pyridin-3-yl)-7-(4-(4-chloro-3-methylphenyl)piperidin-1-yl)-1-methyl-1H-indazole

A mixture of 7-bromo-3-(2,6-dibenzyloxy-3-pyridyl)-1-methyl-indazole (1.00 g, 2.00 mmol, Intermediate AG), 4-(4-chloro-3-methyl-phenyl)piperidine (503 mg, 2.40 mmol, Intermediate AC) and LiHMDS (1 M, 9.99 mL) in toluene (20 mL) was degassed and purged with N₂ 3 times. Then the mixture was stirred at 0° C. for 30 mins under N₂ atmosphere. Next, RuPhos (93.3 mg, 200 mol) and RuPhos Pd G3 (167 mg, 200 mol) was added and the mixture was stirred at 100° C. for 2 hrs. On completion, the reaction mixture was quenched with NH₄Cl (20 mL) at 0° C. and then diluted with water (40 mL) and extracted with ethyl acetate (30 mL×2). The combined organic layers were washed with brine (20 mL×2), dried over Na₂SO₄, filtered and concentrated under reduced pressure to give a residue. The residue was purified by column chromatography (SiO₂, Petroleum ether/Ethyl acetate=40/1 to 20/1) to give the title compound (1 g, 64% yield) as a white solid. LC-MS (ESI⁺) m/z 629.2 (M+H)⁺.

### Step 2—3-(2,6-Bis(benzyloxy)pyridin-3-yl)-1-methyl-7-(4-(3-methyl-4-(4,4,5,5-tetramethyl-1,3,2-dioxaborolan-2-yl)phenyl)piperidin-1-yl)-1H-indazole

A mixture of 7-[4-(4-chloro-3-methyl-phenyl)-1-piperidyl]-3-(2,6-dibenzyloxy-3-pyridyl)-1-methyl-indazole (1.00 g, 1.59 mmol), 4,4,5,5-tetramethyl-2-(4,4,5,5-tetramethyl-1,3,2-dioxaborolan-2-yl)-1,3,2-dioxaborolane (2.02 g, 7.95 mmol, CAS #73183-34-3), XPhos Pd G3 (135 mg, 159 μmol) and KOAc (468 mg, 4.77 mmol) in dioxane (20 mL) was degassed and purged with N₂ 3 times. Then the mixture was stirred at 80° C. for 2 hrs under N₂ atmosphere. The reaction mixture was filtered and concentrated under reduced pressure to give a residue. The crude product was purified by reverse phasereverse phase HPLC (0.1% FA condition) to give the title compound (650 mg, 53% yield) as a white solid. LC-MS (ESI⁺) m/z 721.1 (M+H)⁺.

### Step 3—3-(1-Methyl-7-(4-(3-methyl-4-(4,4,5,5-tetramethyl-1,3,2-dioxaborolan-2-yl)phenyl)piperidin-1-yl)-1H-indazol-3-yl)piperidine-2,6-dione

To a solution of 3-(2,6-dibenzyloxy-3-pyridyl)-1-methyl-7-[4-[3-methyl-4-(4,4,5,5-tetramethyl-1,3,2-dioxaborolan-2-yl)phenyl]-1-piperidyl]indazole (650 mg, 902 μmol) in THF (10 mL) was added Pd/C (10 wt %, 960 mg) under N₂ atmosphere. The suspension was degassed and purged with H₂ 3 times. The mixture was then stirred under H₂ (40 Psi) at 30° C. for 12 hrs. On completion, the reaction mixture was filtered and concentrated under reduced pressure to give title compound (480 mg) as a white solid. LC-MS (ESI⁺) m/z 543.1 (M+H)⁺.

### 3-(1H-1,2,3-triazol-1-yl)propanoic acid (Intermediate AI)

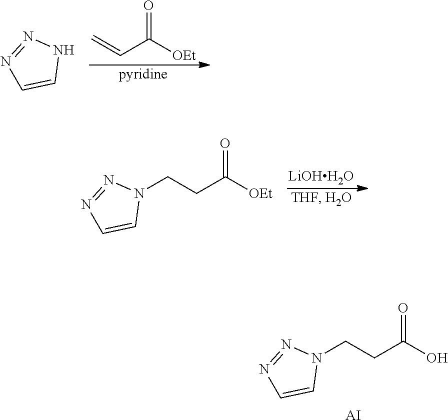

### Step 1—Ethyl 3-(1H-1,2,3-triazol-1-yl)propanoate

To a solution of 1H-1,2,3-triazole (30.0 g, 434 mmol, 25.2 mL, CAS #288-35-7) in ethyl acrylate (43.5 g, 434 mmol, 47.2 mL, CAS #140-88-5) was added pyridine (3.44 g, 43.4 mmol, 3.51 mL). The mixture was then stirred at 90° C. for 4 h. On completion, the mixture was concentrated under vacuum to give the crude product. The crude product was purified by reverse phasereverse phase column (C18, 40 g; condition: water/acetonitrile=1/0 to 0/1, 0.1% formic acid) and lyophilized. The title compound (28.9 g, 39% yield) was obtained as a yellow oil. ¹H NMR (400 MHz, CDCl₃) δ=7.68 (s, 1H), 7.66 (s, 1H), 4.69 (t, J=6.4 Hz, 2H), 4.22-4.07 (m, 2H), 2.97 (t, J=6.4 Hz, 2H), 1.30-1.18 (m, 3H).

### Step 2—3-(1H-1,2,3-triazol-1-yl)propanoic acid

To a solution of ethyl 3-(1H-1,2,3-triazol-1-yl)propanoate (15.0 g, 88.7 mmol) in THF (75.0 mL) and H₂O (75.0 mL) was added LiOH·H₂O (7.44 g, 177 mmol). The mixture was then stirred at 25° C. for 12 hr. On completion, the pH of mixture was adjusted to around 5 by adding HCl (1 M). The mixture was then lyophilized to afford the title compound (19.5 g, 99% yield) as a white solid. ¹H NMR (400 MHz, DMSO-d₆) δ=8.08 (s, 1H), 7.65 (s, 1H), 4.49 (t, J=7.2 Hz, 2H), 2.52-2.48 (m, 2H).

### 1-(5-(4,4,5,5-tetramethyl-1,3,2-dioxaborolan-2-yl)-3,6-dihydropyridin-1(2H)-yl)-3-(1H-1,2,3-triazol-1-yl)propan-1-one ((Intermediate AJ)

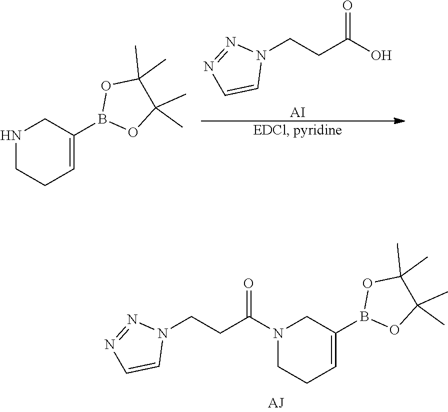

A mixture of 3-(1H-1,2,3-triazol-1-yl)propanoic acid (24.9 g, 114 mmol, Intermediate AI), 5-(4,4,5,5-tetramethyl-1,3,2-dioxaborolan-2-yl)-1,2,3,6-tetrahydropyridine (28.1 g, 114 mmol, HCl, synthesized via Step 1 of Intermediate U), and EDCI (24.1 g, 126 mmol) in pyridine (280 mL) was stirred at 25° C. for 1 h. On completion, the mixture was concentrated and purified by prep-HPLC (column: Welch Ultimate X^{B}—CN 250*70*10 um; mobile phase: [Hexane-EtOH]:B %: 8%-41%, 30 min) to give the title compound (30.0 g, 71% yield) as a yellow gum. LC-MS (ESI⁺) m/z 333.0 (M+H); ¹H NMR (400 MHz, CDCl₃) δ ppm 7.85-7.55 (m, 2H), 6.69 (d, J=2.0 Hz, 1H), 4.77 (dt, J=2.8, 6.0 Hz, 2H), 4.20-3.91 (m, 2H), 3.54-3.48 (m, 2H), 3.10-2.94 (m, 2H), 2.21 (s, 2H), 1.25 (s, 12H).

### 6-(1-(3-(1H-1,2,3-triazol-1-yl)propanoyl)-1,2,5,6-tetrahydropyridin-3-yl)-7-fluoro-N,N-dimethyl-4-(4,4,5,5-tetramethyl-1,3,2-dioxaborolan-2-yl)-1H-indole-2-carboxamide (Intermediate AK)

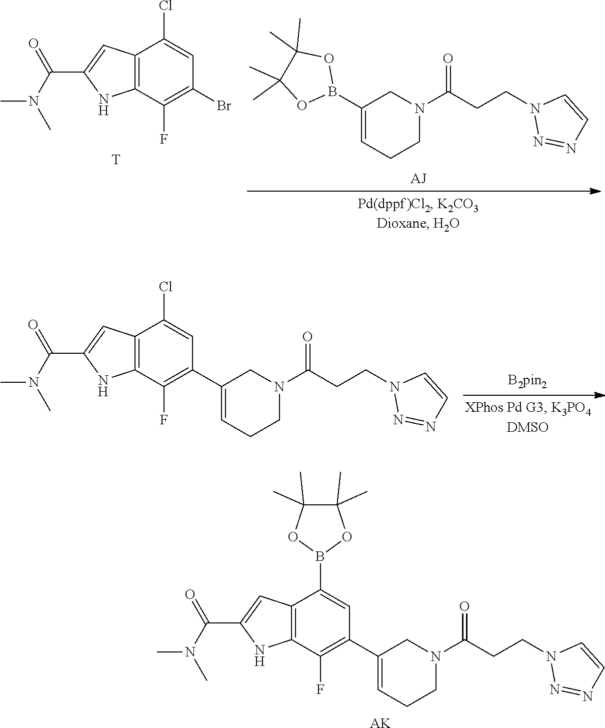

### Step 1—6-(1-(3-(1H-1,2,3-triazol-1-yl)propanoyl)-1,2,5,6-tetrahydropyridin-3-yl)-4-chloro-7-fluoro-N,N-dimethyl-1H-indole-2-carboxamide

A mixture of 6-bromo-4-chloro-7-fluoro-N,N-dimethyl-1H-indole-2-carboxamide (1.20 g, 3.76 mmol, Intermediate T), 1-[5-(4,4,5,5-tetramethyl-1,3,2-dioxaborolan-2-yl)-3,6-dihydro-2H-pyridin-1-yl]-3-(triazol-1-yl)propan-1-one (1.25 g, 3.76 mmol, Intermediate AJ), Pd(dppf)Cl₂ (412 mg, 563 μmol) and K₂CO₃ (1.56 g, 11.3 mmol) in dioxane (8 mL) and H₂O (2 mL) was degassed and purged with N₂ three times. Then the mixture was stirred at 80° C. for 12 hrs under N₂ atmosphere. On completion, the reaction mixture was quenched by addition of H₂O (10 mL), and then extracted with DCM (20 mL×3). The combined organic layers were washed with brine (20 mL), dried over Na₂SO₄, filtered and concentrated under reduced pressure to give a residue. The crude product was purified by reverse phasereverse phase HPLC (0.1% FA condition) to give the title compound (900 mg, 48% yield, FA) as a white solid. LC-MS (ESI⁺) m/z 445.2 (M+H)⁺.

### Step 2—6-(1-(3-(1H-1,2,3-triazol-1-yl)propanoyl)-1,2,5,6-tetrahydropyridin-3-yl)-7-fluoro-N,N-dimethyl-4-(4,4,5,5-tetramethyl-1,3,2-dioxaborolan-2-yl)-1H-indole-2-carboxamide

To a solution of 4-chloro-7-fluoro-N,N-dimethyl-6-[1-[3-(triazol-1-yl)propanoyl]-3,6-dihydro-2H-pyridin-5-yl]-1H-indole-2-carboxamide (200 mg, 450 μmol) and 4,4,5,5-tetramethyl-2-(4,4,5,5-tetramethyl-1,3,2-dioxaborolan-2-yl)-1,3,2-dioxaborolane (228 mg, 899 μmol, CAS #73183-34-3) in DMSO (4 mL) was added KOAc (132 mg, 1.35 mmol) and XPhos Pd G3 (76.1 mg, 89.9 mol). The mixture was the stirred at 80° C. for 12 hrs. On completion, the reaction mixture was concentrated under reduced pressure to remove solvent. The crude product was purified by reverse phase HPLC (0.1% FA condition) to give title compound (80 mg, 27% yield) as a black solid. LC-MS (ESI⁻) m/z 837.2 (M+H)⁺.

### 3-((4-(4-(4-Chlorophenyl)piperidin-1-yl)-3-fluorophenyl)amino)piperidine-2,6-dione (Intermediate AL)

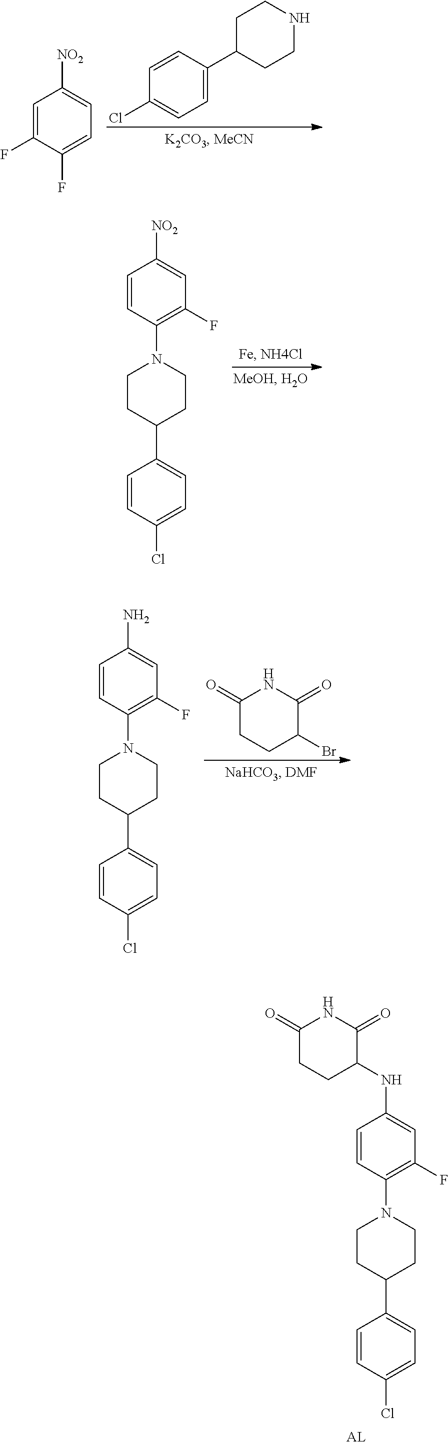

### Step 1—4-(4-Chlorophenyl)-1-(2-fluoro-4-nitrophenyl)piperidine

To a solution of 1,2-difluoro-4-nitro-benzene (2 g, 12.5 mmol, 1.39 mL, CAS #369-34-6) in MeCN (20 mL) was added 4-(4-chlorophenyl)piperidine (3.6 g, 18.8 mmol, CAS #26905-02-2). The mixture was then stirred at 80° C. for 12 hrs. On completion, the reaction mixture diluted with water (100 mL) and extracted with EA (100 mL×3). The combined organic layers were washed with brine (100 mL×2), dried over Na₂SO₄, filtered and concentrated under reduced pressure to give a residue. The residue was purified by column chromatography (SiO₂, Petroleum ether/Ethyl acetate=30/1 to 10/1) to give the title compound (3.3 g, 78% yield) as a yellow solid. LC-MS (ESI⁺) rz 334.9 (M+H)⁺.

### Step 2—4-(4-(4-Chlorophenyl)piperidin-1-yl)-3-fluoroaniline

To a solution of 4-(4-chlorophenyl)-1-(2-fluoro-4-nitro-phenyl)piperidine (2.9 g, 8.6 mmol) in EtOH (30 mL) and H₂O (30 mL) was added Fe (2.4 g, 43.3 mmol) and NH₄Cl (3.7 g, 69.3 mmol). The mixture was then stirred at 25° C. for 12 hrs. On completion, the combined organic layers were washed with brine (100 mL×2), dried over Na₂SO₄, filtered and concentrated under reduced pressure to give the title compound (2.7 g) as a yellow solid. LC-MS (ESI⁺) m/z 305.0 (M+H)⁺.

### Step 3—3-((4-(4-(4-Chlorophenyl)piperidin-1-yl)-3-fluorophenyl)amino)piperidine-2,6-dione

To a solution of 4-[4-(4-chlorophenyl)-1-piperidyl]-3-fluoro-aniline (2.5 g, 8.2 mmol) in DMF (30 mL) was added NaHCO₃ (1.3 g, 16.4 mmol) and 3-bromopiperidine-2,6-dione (3.1 g, 16 mmol, CAS #62595-74-8). The mixture was the stirred at 80° C. for 12 hrs. On completion, the residue was purified by prep-HPLC (0.1% FA) to give the title compound (850 mg, 22% yield, FA) as a green solid. LC-MS (ESI⁺) m/z 416.1 (M+H)⁺.

### 4-Bromo-3-methyl-1-((2-(trimethylsilyl)ethoxy)methyl)-1,3-dihydro-2H-benzo[d]imidazol-2-one (Intermediate AM)

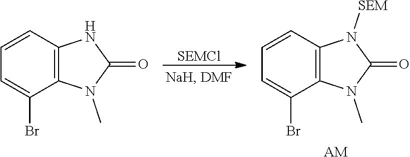

To a solution of 4-bromo-3-methyl-1H-benzimidazol-2-one (15 g, 66.0 mmol, CAS #913297-44-6) in DMF (150 mL) was added NaH (3.96 g, 99.1 mmol, 60% dispersion in mineral oil) at 0° C. After addition, the mixture was stirred 0° C. for 0.5 hours, and then SEM-C1 (13.2 g, 79.3 mmol, 14.0 mL, CAS #76513-69-4) was added dropwise at 0° C. The resulting mixture was stirred at 0° C. for 1 hr, then stirred at 20° C. for 12 h. On completion, the reaction mixture was quenched by addition of sat. NH₄Cl (200 mL) at 0° C., and then filtered, and the filtrate was extracted with DCM (250 mL×3). The combined organic layers were washed with sat. NaCl (200 mL×3), dried over Na₂SO₄, filtered and concentrated under reduced pressure to give a residue. The residue was purified by column chromatography (SiO₂, Petroleum ether:Ethyl acetate=1/0 to 9/1) to give the title compound (7.4 g, 30% yield) as a colorless oil. LC-MS (ESI⁺) m/z 329.0 (M-27)⁺.

### 7-(4-(4-chlorophenyl)piperidin-1-yl)-1-methyl-1,3-dihydro-2H-benzo[d]imidazol-2-one (Intermediate AN)

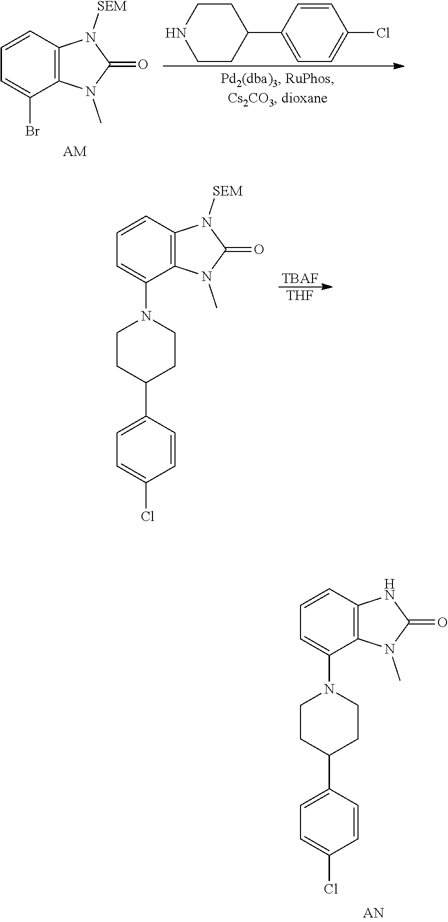

### Step 1—4-(4-(4-chlorophenyl)piperidin-1-yl)-3-methyl-1-((2-(trimethylsilyl)ethoxy)methyl)-1,3-dihydro-2H-benzo[d]imidazol-2-one

A mixture of 4-bromo-3-methyl-1-(2-trimethylsilylethoxymethyl)benzimidazol-2-one (1 g, 2.80 mmol, Intermediate AM), 4-(4-chlorophenyl)piperidine (1.64 g, 8.40 mmol, CAS #26905-02-2), Cs₂CO₃ (2.74 g, 8.40 mmol), Pd₂(dba)₃ (256 mg, 279 μmol) and RuPhos (261 mg, 559 mol) in dioxane (15 mL) was degassed and purged with N₂ three times. Then the mixture was stirred at 100° C. for 12 hrs under N₂ atmosphere. On completion, the reaction mixture was diluted with water (20 mL) and extracted with ethyl acetate (3×10 mL). The combined organic layers were washed with brine (30 mL) and dried over anhydrous sodium sulfate, filtered and the filtrate was concentrated in vacuo to give the crude residue. The residue was purified by column chromatography (SiO₂, Petroleum ether:Ethyl acetate=1/0 to 0/1) to give the title compound (0.8, 30% yield) as a yellow solid. LC-MS (ESI⁺) m/z 472.2 (M+H)⁺.

### Step 2—7-(4-(4-Chlorophenyl)piperidin-1-yl)-1-methyl-1,3-dihydro-2H-benzo[d]imidazol-2-one

To a solution of 4-[4-(4-chlorophenyl)-1-piperidyl]-3-methyl-1-(2-trimethylsilylethoxymethyl)benzimidazol-2-one (900 mg, 1.91 mmol) in THF (10 mL) was added TBAF (1 M, 18.00 mL) at 25° C., then the mixture was stirred at 80° C. for 16 hrs. On completion, the reaction mixture was quenched by addition of ice-water (50 mL) at 25° C., and then diluted with EtOAc (50 mL) and extracted with EtOAc (50 mL×3). The combined organic layers were washed with brine (50 mL×2), dried over Na₂SO₄, filtered and concentrated under reduced pressure to give a residue. The residue was purified by column chromatography (SiO₂. Petroleum ether/DCM/Ethyl acetate=1/1/5 to 1/4/5) to give the title compound (400 mg, 49% yield) as a yellow oil. LC-MS (ESI⁺) m/z 342.0 (M+H)⁺.

### 1-(4-Methoxybenzyl)-2,6-dioxopiperidin-3-yl trifluoromethanesulfonate (Intermediate AO)

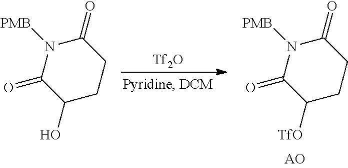

To a solution of 3-hydroxy-1-(4-methoxybenzyl)piperidine-2,6-dione (5 g, 20.0 mmol, CAS #2357109-89-6) in DCM (50 mL) was added pyridine (3.17 g, 40.1 mmol, 3.24 mL) and Tf₂O (8.49 g, 30.1 mmol, 4.96 mL). The mixture was then stirred at 0° C. for 2 hrs. On completion, the reaction mixture was concentrated under reduced pressure to give a residue. The residue was purified by column chromatography (SiO₂, Petroleum ether/Ethyl acetate=1/0 to 4/1) to give the title compound (5.8 g, 74% yield) as a light yellow oil. LC-MS (ESI⁺) m/z 404.0 (M+Na)⁺.

### 3-(4-(4-(4-Chlorophenyl)piperidin-1-yl)-3-methyl-2-oxo-2,3-dihydro-1H-benzo[d]imidazol-1-yl)piperidine-2,6-dione (Intermediate AP)

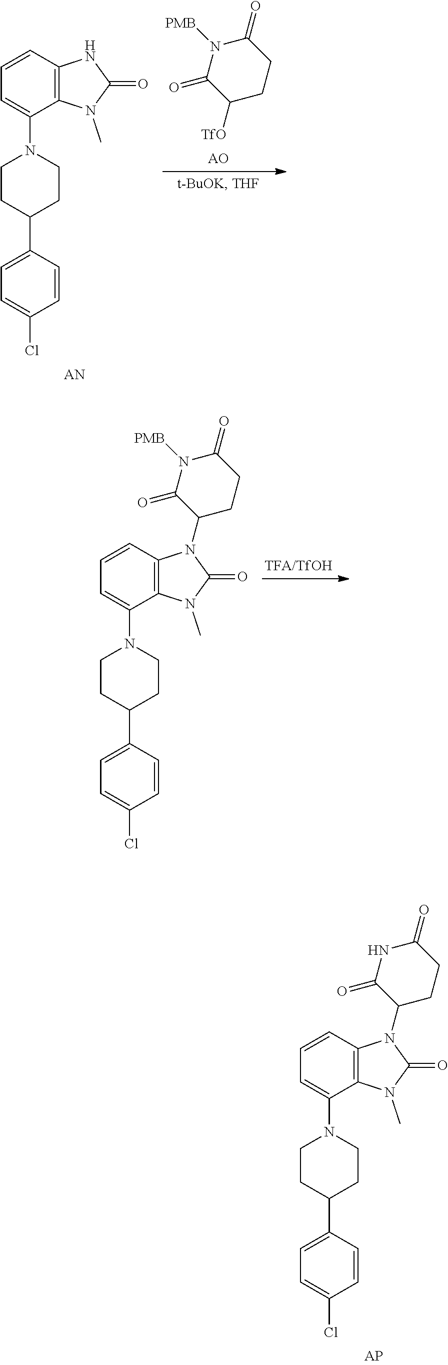

### Step 1—3-(4-(4-(4-Chlorophenyl)piperidin-1-yl)-3-methyl-2-oxo-2,3-dihydro-1H-benzo[d]imidazol-1-yl)-1-(4-methoxybenzyl)piperidine-2,6-dione

To a solution of 7-(4-(4-chlorophenyl)piperidin-1-yl)-1-methyl-1,3-dihydro-2H-benzo[d]imidazol-2-one (350 mg, 1.02 mmol, Intermediate AN) in THF (15 mL) was added t-BuOK (1 M, 1.54 mL) dropwise over 10 min at 0° C. The mixture was then stirred at 0° C. for 1 h, then 1-(4-methoxybenzyl)-2,6-dioxopiperidin-3-yl trifluoromethanesulfonate (781 mg, 2.05 mmol, Intermediate AO) in THF (5 mL) was added to the solution at 0° C. Then the mixture was stirred at 25° C. for 15 hrs. On completion, the reaction mixture was quenched by addition of FA (1 mL) at 0° C., adjusted pH to 3˜4 with FA, and then diluted with H₂O (50 mL) and extracted with EA (50 mL×2). The combined organic layers were dried over Na₂SO₄, filtered and the filtrate was concentrated under reduced pressure to give a residue. The residue was purified by column chromatography (SiO₂. Petroleum ether/Ethyl acetate=10/1 to 3/1) to give the title compound (540 mg, 74% yield) as a white solid. LC-MS (ESI⁺) m/z 573.3 (M+H)⁺.

### Step 2—3-(4-(4-(4-chlorophenyl)piperidin-1-yl)-3-methyl-2-oxo-2,3-dihydro-1H-benzo[d]imidazol-1-yl)piperidine-2,6-dione

To a solution of 3-[4-[4-(4-chlorophenyl)-1-piperidyl]-3-methyl-2-oxo-benzimidazol-1-yl]-1-[(4-methoxyphenyl)methyl]piperidine-2,6-dione (490 mg, 855 μmol) in TFA (10 mL) was added TfOH (8.31 g, 4.90 mL). The mixture was then stirred at 50° C. for 16 hrs. On completion, the reaction mixture was concentrated under reduced pressure to remove solvent and the residue was purified by prep-HPLC (FA condition) to give the title compound (280 mg, 72% yield) as a yellow solid. LC-MS (ESI⁺) m/z 453.0 (M+H)⁺.

### 3-((4-(4-(4-Chlorophenyl)piperazin-1-yl)-5-fluoro-2-methoxyphenyl)amino)piperidine-2,6-dione (Intermediate AQ)

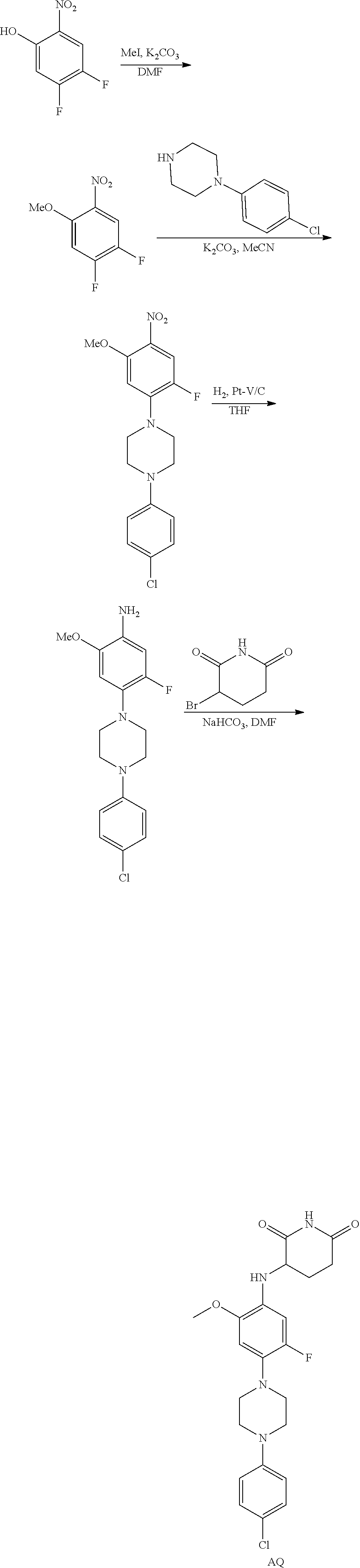

### Step 1—1,2-Difluoro-4-methoxy-5-nitrobenzene

To a mixture of 4,5-difluoro-2-nitrophenol (5.00 g, 28.6 mmol, CAS #55346-97-9) and K₂CO₃ (11.8 g, 85.7 mmol) in DMF (30.0 mL) was added Mel (12.2 g, 85.7 mmol). The mixture was then stirred at 20° C. for 16 hr. On completion, the reaction mixture was poured into water (300 mL), and extracted with EA (50 mL×3). The combined organic layers were washed with brine (100 mL), dried over anhydrous Na₂SO₄, filtered and concentrated under reduced pressure to give the title compound (5.35 g, 99% yield) as a yellow solid. ¹H NMR (400 MHz, CDCl₃) δ=7.87 (dd, J=8.4, 9.6 Hz, 1H), 6.94 (dd, J=6.4, 11.2 Hz, 1H), 3.97 (s, 3H).

### Step 2—1-(4-Chlorophenyl)-4-(2-fluoro-5-methoxy-4-nitrophenyl)piperazine

To a mixture of 1,2-difluoro-4-methoxy-5-nitrobenzene (2.80 g, 14.8 mmol) and 1-(4-chlorophenyl) piperazine (3.49 g, 17.8 mmol, CAS #38212-33-8) in MeCN (30.0 mL) was added K₂CO₃ (4.09 g, 29.6 mmol). The mixture was then stirred at 80° C. for 2 hrs. On completion, water (300 mL) was added to the mixture which was then filtered. The filter cake was washed with MeCN (20.0 mL) and dried to give the title compound (3.80 g, 70% yield) as a yellow solid. LC-MS (ESI⁺) m/z 366.0 (M+H)⁺; ¹H NMR (400 MHz, DMSO-d₆) δ=7.87 (d, J=13.6 Hz, 1H), 7.27 (d, J=9.2 Hz, 2H), 7.01 (d, J=9.2 Hz, 2H), 6.73 (d, 0.1=7.6 Hz, 1H), 3.95 (s, 3H), 3.55-3.39 (m, 4H), 3.32-3.27 (m, 4H).

### Step 3—4-(4-(4-Chlorophenyl)piperazin-1-yl)-5-fluoro-2-methoxyaniline

To a solution of 1-(4-chlorophenyl)-4-(2-fluoro-5-methoxy-4-nitrophenyl)piperazine (3.50 g, 9.57 mmol) in THF (150 mL) was added Pt—V/C (3.50 g). The mixture was then stirred at 20° C. for 12 h under H₂ (15 psi). On completion, the reaction mixture was filtered and the filtrated was concentrated to give the crude product. The crude product was purified by reverse phase HPLC (0.1% FA condition) to give the title compound (1.9 g, 59% yield) as an off-white solid. ¹H NMR (400 MHz, DMSO-d₆) δ=7.26 (d, J=8.8 Hz, 2H), 7.00 (d, J=8.8 Hz, 2H), 6.82 (d, J=12.8 Hz, 1H), 6.72 (d, J=8.0 Hz, 1H), 3.84 (s, 3H), 3.27 (d, J=5.2 Hz, 4H), 3.13 (s, 4H).

### Step 4—3-((4-(4-(4-Chlorophenyl)piperazin-1-yl)-5-fluoro-2-methoxyphenyl)amino)piperidine-2,6-dione

To a mixture of 4-(4-(4-chlorophenyl)piperazin-1-yl)-5-fluoro-2-methoxyaniline (1.00 g, 2.98 mmol) and 3-bromopiperidine-2,6-dione (858 mg, 4.47 mmol) in DMF (10.0 mL) was added NaHCO₃ (500 mg, 5.96 mmol). The mixture was stirred at 80° C. for 16 hours. The reaction mixture was poured in to sat. NH₄Cl aq. (50.0 mL) at 0° C., diluted with water (50.0 mL), filtered and the filter cake was dried to give the crude. The crude was triturated with EA (150 mL) at 25° C. for 30 min, filtered and the filtrate was concentrated to give the title compound (0.60 g, 45% yield) as a sage green solid. LC-MS (ESI⁺) n/z 447.1 (M+H)⁺. ¹H NMR (400 MHz, DMSO-d₆) δ=10.85 (s, 1H), 7.25 (d, J=8.8 Hz, 2H), 6.99 (d, J=8.8 Hz, 2H), 6.75-6.49 (m, 2H), 5.45-4.81 (m, 1H), 4.27 (dd, J=4.8, 12.4 Hz, 1H), 3.80 (s, 3H), 3.30-3.22 (m, 4H), 3.06 (d, J=4.4 Hz, 4H), 2.91-2.71 (m, 1H), 2.60-2.52 (m, 1H), 2.22-2.08 (m, 1H), 2.04-1.84 (m, 1H).

### 6-(1-(3-(1H-pyrazol-1-yl)propanoyl)-1,2,5,6-tetrahydropyridin-3-yl)-7-fluoro-N,N-dimethyl-4-(4,4,5,5-tetramethyl-1,3,2-dioxaborolan-2-yl)-1H-indole-2-carboxamide (Intermediate AR)

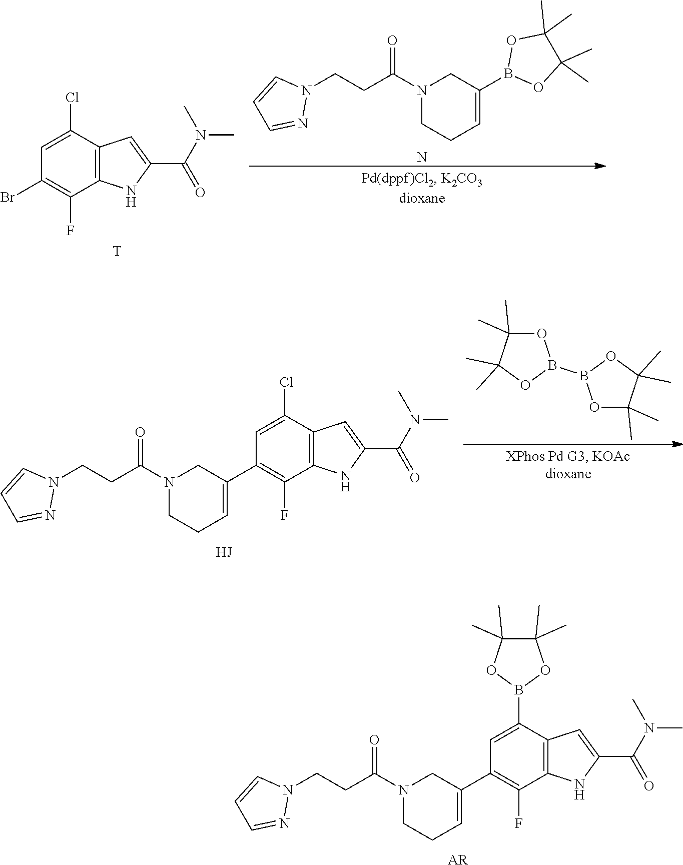

### Step 1—6-(1-(3-(1H-pyrazol-1-yl)propanoyl)-1,2,5,6-tetrahydropyridin-3-yl)-4-chloro-7-fluoro-N,N-dimethyl-1H-indole-2-carboxamide (Intermediate HJ)

To a solution of 3-(1H-pyrazol-1-yl)-1-(5-(4,4,5,5-tetramethyl-1,3,2-dioxaborolan-2-yl)-3,6-dihydropyridin-1(2H)-yl)propan-1-one (15.6 g, 46.9 mmol, Intermediate N) and 6-bromo-4-chloro-7-fluoro-N,N-dimethyl-1H-indole-2-carboxamide (15 g, 46.9 mmol, Intermediate T) in dioxane (100 mL) and H₂O (20 mL) was added K₂CO₃ (19.5 g, 141 mmol) and Pd(dppf)Cl₂·CH₂Cl₂ (3.83 g, 4.69 mmol) at 25° C. Then the mixture was stirred at 80° C. for 2 hrs. On completion, the reaction mixture was concentrated in vacuo to give the crude residue. The residue was purified by column chromatography (SiO₂, Petroleum ether/Ethyl acetate=l/1 to DCM:MeOH=30:1) to give the title compound (14.7 g, 67% yield) as a yellow solid. LC-MS (ESI⁺) m/z 444.2 (M+H)⁺.

### Step 2—6-(1-(3-(1H-pyrazol-1-yl)propanoyl)-1,2,5,6-tetrahydropyridin-3-yl)-7-fluoro-N,N-dimethyl-4-(4,4,5,5-tetramethyl-1,3,2-dioxaborolan-2-yl)-1H-indole-2-carboxamide

To a solution of 6-(1-(3-(1H-pyrazol-1-yl)propanoyl)-1,2,5,6-tetrahydropyridin-3-yl)-4-chloro-7-fluoro-N,N-dimethyl-1H-indole-2-carboxamide (12.8 g, 28.8 mmol) and 4,4,4′,4′,5,5,5′,5′-octamethyl-2,2′-bi(1,3,2-dioxaborolane) (14.6 g, 57.7 mmol) in dioxane (50 mL) was added XPhos Pd G3 (2.44 g, 2.88 mmol) and KOAc (8.49 g, 86.5 mmol) at 25° C. Then the mixture was stirred at 80° C. for 1 hr. On completion, the reaction mixture was concentrated in vacuo to give the crude residue. The residue was purified by column chromatography (SiO₂, Petroleum ether/Ethyl acetate=1/1 to DCM:MeOH=20:1) to give the title compound (11 g, 64% yield) as a yellow solid. LC-MS (ESI⁺) m/z 536.2 (M+H)⁺.

### 1-(5-chloro-2-(dimethylcarbamoyl)benzofuran-7-yl)azetidine-3-carboxylic acid (Intermediate AS)

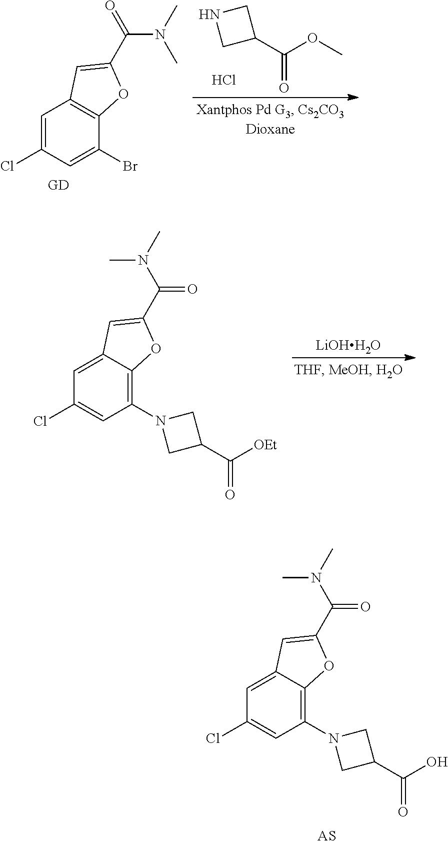

### Step 1—Methyl 1-(5-chloro-2-(dimethylcarbanoyl)benzofuran-7-yl)azetidine-3-carboxylate

A mixture of 7-bromo-5-chloro-N,N-dimethylbenzofuran-2-carboxamide (2 g, 6.61 mmol, Intermediate GD) methyl azetidine-3-carboxylate hydrochloride (1.00 g, 6.61 mmol, CAS #100202-39-9), Cs₂CO₃ (6.46 g, 19.8 mmol) and [2-(2-aminophenyl)phenyl]-methylsulfonyloxy-palladium (5-diphenylphosphanyl-9,9-dimethyl-xanthen-4-yl)-diphenyl-phosphane (626 mg, 661 mol) in dioxane (20 mL) was degassed and purged with N₂ three times. Then the mixture was stirred at 100° C. for 12 hrs under N₂ atmosphere. On completion, the reaction mixture was diluted with water (20 mL) and extracted with dichloromethane (2×20 mL). The combined organic layers were washed with brine (20 mL) and dried over anhydrous sodium sulfate, filtered and the filtrate was concentrated in vacuo to give the crude residue. The residue was purified by column chromatography (SiO₂, Petroleum ether:Ethyl acetate=1/0 to 1/1) to give the title compound (1.6 g, 72% yield) as a white solid. LC-MS (ESI⁺) m/z 337.0 (M+H)⁺.

### Step 2—1-(5-chloro-2-(dimethylcarbamoyl)benzofuran-7-yl)azetidine-3-carboxylic acid

A solution of methyl 1-(5-chloro-2-(dimethylcarbamoyl)benzofuran-7-yl)azetidine-3-carboxylate (400 mg, 1.19 mmol), and LiOH·H₂O (125 mg, 2.97 mmol) in THF (2 mL), MeOH (1 mL) and H₂O (1 mL) was degassed and purged with N₂ three times. Then the mixture was stirred at 25° C. for 1 h under N₂ atmosphere. On completion, the reaction mixture was quenched by addition of H₂O (2 mL), and then extracted with DCM (3 mL×3). The combined aqueous phase was treated with NH₄Cl to adjusted pH=6˜7, then the mixture was concentrated under reduced pressure to give a residue. The residue was purified by prep-HPLC (neutral condition, H₂O(NH₄CO₃)/ACN; 10%-60%) to give the title compound (274 mg, 72% yield) as a white solid. LC-MS (ESI⁺) m/z 323.1 (M+H)⁺.

### 3-(3-(4-(Piperidin-4-ylmethyl)piperazin-1-yl)phenyl)piperidine-2,6-dione (Intermediate AT)

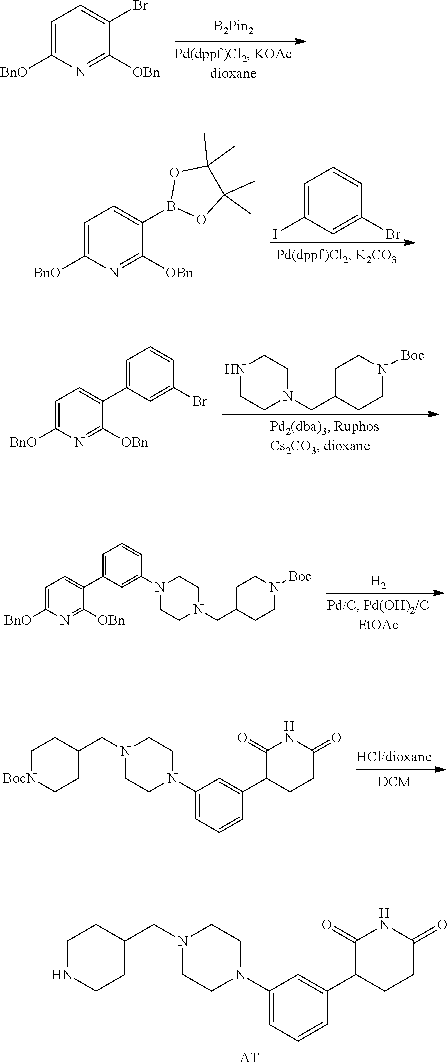

### Step 1—2,6-Dibenzyloxy-3-(4,4,5,5-tetramethyl-1,3,2-dioxaborolan-2-yl)pyridine

To a solution of 2,6-dibenzyloxy-3-bromo-pyridine (40 g, 110 mmol, CAS #16727-47-2), 4,4,5,5-tetramethyl-2-(4,4,5,5-tetramethyl-1,3,2-dioxaborolan-2-yl)-1,3,2-dioxaborolane (80 g, 300 mmol) in dioxane (100 mL) was added Pd(dppf) Cl₂ (7.91 g, 10.8 mmol) and KOAc (31.81 g, 324.1 mmol). Then the mixture was stirred at 120° C. for 12 hr under N₂. On completion, the reaction mixture was extracted with water (200 mL) and ethyl acetate (250 mL) three times. Then the organic phase was concentrated on a rotary evaporator to give the title compound (88 g) as yellow oil. LC-MS (ESI⁺) m/z 418.2 (M+H)⁺.

### Step 2—2,6-Dibenzyloxy-3-(3-bromophenyl)pyridine

To a mixture of 1-bromo-3-iodo-benzene (6.78 g, 24.0 mmol, 3.05 mL, CAS #591-18-4) and 2,6-dibenzyloxy-3-(4,4,5,5-tetramethyl-1,3,2-dioxaborolan-2-yl)pyridine (10 g, 20 mmol) in dioxane (20 mL) and H₂O (5 mL) was added Pd(dppf) Cl₂ (1.75 g, 2.40 mmol) and K₂CO₃ (9.94 g, 71.9 mmol). Then the mixture was stirred at 80° C. for 2 hr under N₂. On completion, the reaction mixture was washed with water (200 mL) and extracted with ethyl acetate (200 mL×3). Then the organic phase was concentrated on a rotary evaporator. The residue was purified by column chromatography (SiO₂, Petroleum ether/Ethyl acetate=21/1 to 30/1) to give the title compound (5 g) as a yellow oil. LC-MS (ESI⁺) m/z 448.0 (M+H)⁺.

### Step 3—Tert-butyl 4-[[4-[3-(2,6-dibenzyloxy-3-pyridyl)phenyl]piperazin-1-yl]methyl]piperidine-1-carboxylate

To a solution of 2,6-dibenzyloxy-3-(3-bromophenyl) pyridine (3.7 g, 8.3 mmol) and tert-butyl 4-(piperazin-1-ylmethyl)piperidine-1-carboxylate (2.82 g, 9.95 mmol. CAS #381722-48-1) in dioxane (10 mL) was added Pd₂ (dba)₃ (227.73 mg, 248.69 μmol), Cs₂CO₃ (8.10 g, 24.9 mmol). RuPhos (386.83 mg, 828.97 μmol). Then the mixture was stirred at 80° C. for 2 h under N₂. On completion, the reaction mixture was washed with water (100 mL) and extracted with ethyl acetate (100 mL×3). Then the organic phase was concentrated on a rotary evaporator. The residue was purified by column chromatography (SiO₂, Petroleum ether/Ethyl acetate=21/1 to 30/1) to give the title compound (5 g) as a yellow oil. LC-MS (ESI⁺) m/z 649.5 (M+H)⁺.

### Step 4—Tert-butyl 4-[[4-[3-(2,6-dioxo-3-piperidyl)phenyl]piperazin-1-yl]methyl]piperidine-1-carboxylate

To a solution of tert-butyl 4-[[4-[3-(2,6-dibenzyloxy-3-pyridyl)phenyl]piperazin-1-yl]methyl]piperidine-1-carboxylate (2.1 g, 3.2 mmol) in EA (20 mL) was added Pd/C (1 g, 10 wt %) and Pd(OH)₂ (909.06 mg, 3.24 mmol, 50 wt %). Then the mixture was stirred at 25° C. for 12 h under N₂. On completion, the mixture was filtered with EA (100 mL). The crude product was purified by reverse phase HPLC (0.1% FA condition) to give the title compound (520 mg, 34% yield) as a yellow oil. ¹H NMR (400 MHz, DMSO-d₆) δ=10.80 (s, 1H), 8.15 (s, 1H), 7.16 (t, J=7.8 Hz, 1H), 6.85-6.77 (m, 2H), 6.62 (d, J=7.5 Hz, 1H), 3.93 (br d, J=11.8 Hz, 2H), 3.76 (dd, J=4.9, 11.2 Hz, 1H), 3.18-3.11 (m, 4H), 2.77-2.53 (m, 6H), 2.49-2.41 (m, 1H), 2.20 (br d, J=6.8 Hz, 3H), 2.07-1.96 (m, 1H), 1.70 (br d, J=12.1 Hz, 4H), 1.39 (s, 9H), 0.96 (br dd, J=2.3, 12.1 Hz, 2H)

### Step 5—3-(3-(4-(Piperidin-4-ylmethyl)piperazin-1-yl)phenyl)piperidine-2,6-dione

To a solution of tert-butyl 4-((4-(3-(2,6-dioxopiperidin-3-yl)phenyl)piperazin-1-yl)methyl)piperidine-1-carboxylate (100 mg, 212 μmol) in DCM (1 mL) was added HCl/dioxane (4 M, 1.06 mL) The mixture was then stirred at 25° C. for 2.5 hr. On completion, the reaction mixture was concentrated under reduced pressure to give the title compound (93 mg, HCl) as a white solid. LC-MS (ESI⁺) m/z 371.2 (M+H)⁺.

### 5-chloro-7-(3-(4-((4-(3-(2,6-dioxopiperidin-3-yl)phenyl)piperazin-1-yl)methyl)piperidine-1-carbonyl)azetidin-1-yl)-N,N-dimethylbenzofuran-2-carboxamide (Intermediate AU)

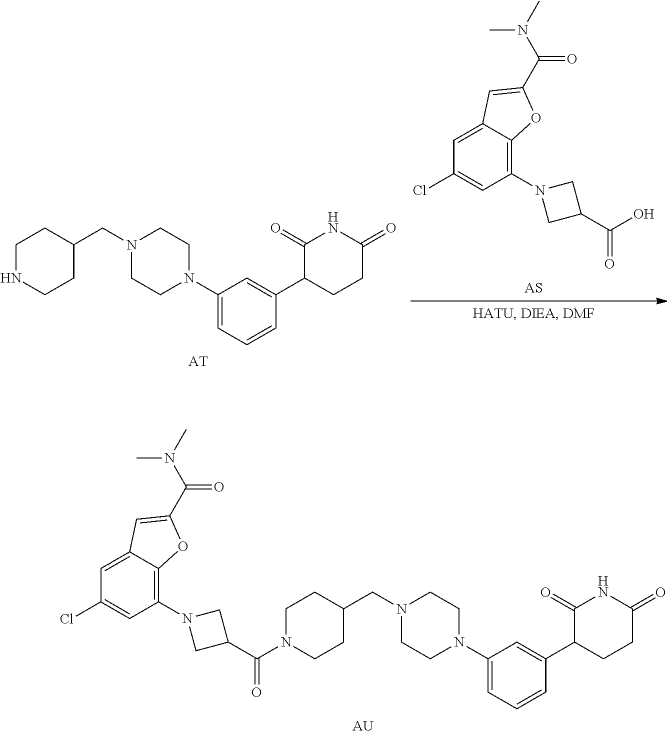 [SMILES: O=C1CCC(c2cccc(N3CCN(CC4CCNCC4)CC3)c2)C(=O)N1 ; CN(C)C(=O)c1cc2cc(Cl)cc(N3CC(C(=O)O)C3)c2o1 ; "AT" ; CN(C)C(=O)c1cc2cc(Cl)cc(N3CC(C(=O)N4CCC(CN5CCN(c6cccc(C7CCC(=O)NC7=O)c6)CC5)CC4)C3)c2o1 ; "AU"]

A solution of 3-(3-(4-(piperidin-4-ylmethyl)piperazin-1-yl)phenyl)piperidine-2,6-dione (86 mg, 211 μmol, HCl, Intermediate AT) and DIEA (72.8 mg, 564 μmol, 98.2 μL) in DMF (0.8 mL) was stirred at 25° C. Then HATU (69.6 mg, 183 μmol) and 1-(5-chloro-2-(dimethylcarbamoyl)benzofuran-7-yl)azetidine-3-carboxylic acid (45.5 mg, 141 μmol, Intermediate AS) was added to the mixture under 0° C. and the mixture was stirred at 0° C. for 1 h. On completion, the reaction mixture was filtered. The crude product was purified by reverse phase HPLC (0.1% FA condition) to give the title compound (84 mg, 84% yield) as a white solid. ¹H NMR (400 MHz, DMSO-d₆) δ=10.80 (s, 1H), 7.31 (s, 1H), 7.23-7.13 (m, 1H), 7.07 (d, J=1.6 Hz, 1H), 6.91-6.77 (m, 2H), 6.71-6.57 (m, 1H), 6.43 (d, J=2.0 Hz, 1H), 4.48-4.26 (m, 3H), 4.19 (q, J=6.8 Hz, 2H), 3.92-3.85 (m, 1H), 3.79-3.74 (m, 1H), 3.64 (br dd, J=2.0, 12.0 Hz, 1H), 3.25 (br s, 6H), 3.15-2.99 (m, 8H), 2.62 (br dd, J=6.0, 10.8 Hz, 4H), 2.30-2.09 (m, 3H), 2.02 (dt, J=4.8, 8.8 Hz, 1H), 1.83-1.74 (m, 2H), 1.15-0.90 (m, 2H), 1.09-0.89 (m, 1H).

### 6-(1-Acetylpiperidin-3-yl)-7-fluoro-N,N-dimethyl-4-(4,4,5,5-tetramethyl-1,3,2-dioxaborolan-2-yl)-1H-indole-2-carboxamide (Intermediate AU)

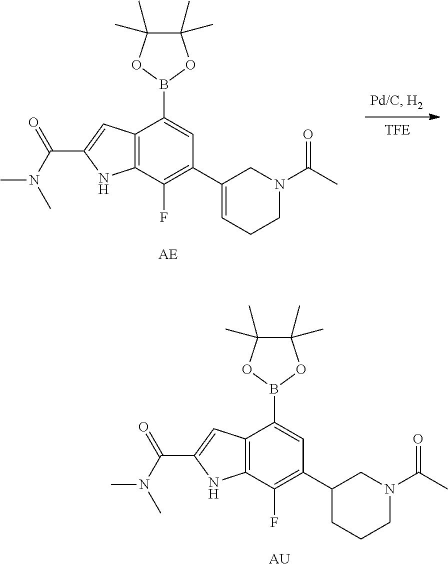

To a solution of 6-(1-acetyl-1,2,5,6-tetrahydropyridin-3-yl)-7-fluoro-N,N-dimethyl-4-(4,4,5,5-tetramethyl-1,3,2-dioxaborolan-2-yl)-1H-indole-2-carboxamide (2.4 g, 5.27 mmol, Intermediate AE) in TFE (20 mL) was added Pd/C (2 g, 10 wt %) under N₂ atmosphere. The suspension was degassed and purged with H₂ three times. Then the mixture was stirred under H₂ (15 psi) at 40° C. for 3 hrs. On completion, the reaction was filtered through kieselguhr very carefully, and the filtrate was concentrated in vacuo to give a residue and the catalyst was recollected. The crude residue was purified by reverse phase HPLC (0.1% FA condition) to give the title compound (3.5 g) as a yellow solid. LCMS (ESI⁻) m/z 458.3. (M+H)⁺.

### (S)-6-(1-acetylpiperidin-3-yl)-7-fluoro-N,N-dimethyl-4-(4,4,5,5-tetramethyl-1,3,2-dioxaborolan-2-yl)-1H-indole-2-carboxamide (Intermediate AV) & (R)-6-(1-acetylpiperidin-3-yl)-7-fluoro-N,N-dimethyl-4-(4,4,5,5-tetramethyl-1,3,2-dioxaborolan-2-yl)-1H-indole-2-carboxamide (Intermediate AW)

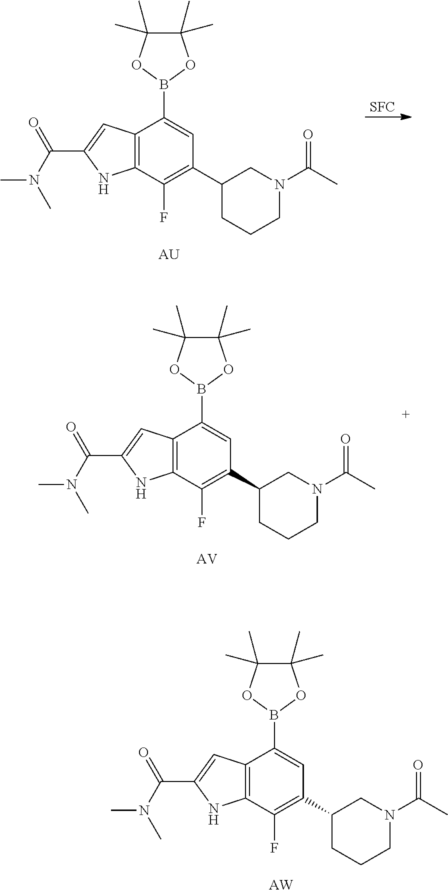

6-(1-Acetylpiperidin-3-yl)-7-fluoro-N,N-dimethyl-4-(4,4,5,5-tetramethyl-1,3,2-dioxaborolan-2-yl)-1H-indole-2-carboxamide (3.5 g, Intermediate AU) was purified by reverse phase HPLC (column: REGIS (R,R)WHELK-O1(250 mm*25 mm, 10 um); mobile phase: [CO₂-MeOH(0.1% NH₃H₂O)]; B %:40%, isocratic elution mode) to give (S)-6-(1-acetylpiperidin-3-yl)-7-fluoro-N,N-dimethyl-4-(4,4,5,5-tetramethyl-1,3,2-dioxaborolan-2-yl)-1H-indole-2-carboxamide (650 mg, 26% yield) as a white solid (LCMS (ESI⁺) m/z 457.9. (M+H)⁺) and (R)-6-(1-acetylpiperidin-3-yl)-7-fluoro-N,N-dimethyl-4-(4,4,5,5-tetramethyl-1,3,2-dioxaborolan-2-yl)-1H-indole-2-carboxamide (600 mg, 26% yield) as a yellow solid (LCMS (ESI⁺) m/z 458.2. (M+H)⁺). The absolute stereochemistry of the enantiomers was assigned arbitrarily.

### 3-(4-(4-(4-chloro-3-methoxyphenyl)piperidin-1-yl)-3-methyl-2-oxo-2,3-dihydro-1H-benzo[d]imidazol-1-yl)piperidine-2,6-dione (Intermediate AX)

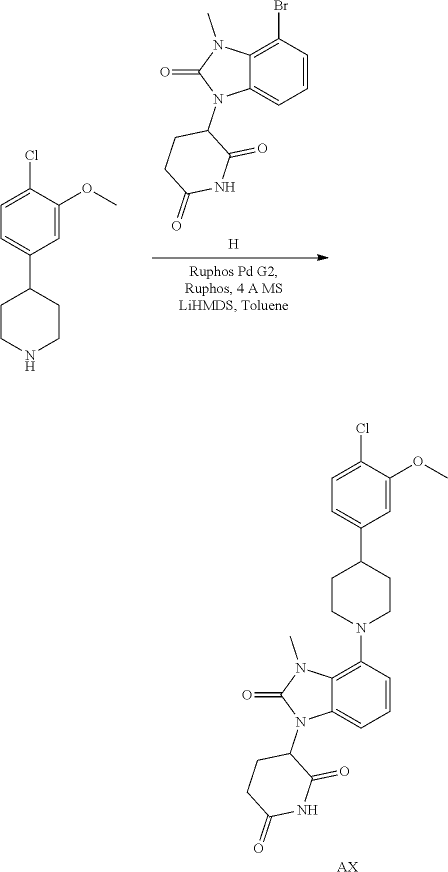

To a solution of 3-(4-bromo-3-methyl-2-oxo-2,3-dihydro-1H-benzo[d]imidazol-1-yl)piperidine-2,6-dione (700 mg, 2.07 mmol, Intermediate H) in anhydrous toluene (10 mL) was added 4-(4-chloro-3-methoxyphenyl)piperidine (450 mg, 1.72 mmol, HCl, CAS #2168546-59-4) and 4A molecular sieves (2 g) at 0° C. Then LiHMDS (1 M, 10.3 mL) was added slowly under nitrogen atmosphere, followed by RuPhos (120 mg, 257 μmol) and RuPhos Pd G2 (133 mg, 172 μmol). Then the reaction was stirred at 100° C. for 1 hr under nitrogen atmosphere. On completion, the mixture was quenched by HCO₂H until the pH=6, then concentrated to give a crude product. The crude residue was purified by reverse phase HPLC (0.8 g/L ammonium bicarbonate) to give the title compound (450 mg, 50% yield) as a yellow solid. LC-MS (ESI⁺) m/z 483.2 (M+H)⁺.

### 7-bromo-5-chloro-4-fluoro-N,N-dimethyl-benzofuran-2-carboxamide (Intermediate AY)

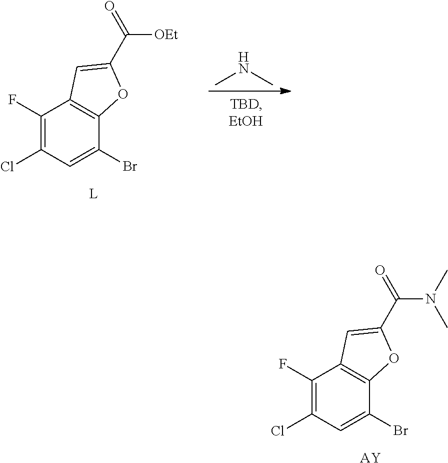

To a solution of ethyl 7-bromo-5-chloro-4-fluoro-benzofuran-2-carboxylate (1 g, 3 mmol, Intermediate L) in EtOH (10 mL) was added 3,4,6,7,8,9-hexahydro-2H-pyrimido[1,2-a]pyrimidine (85.7 mg, 616 μmol, CAS #5807-14-7). Then N-methylmethanamine (2 M in TIF, 2.31 mL, CAS #124-40-3) was added dropwise. Then the mixture was stirred at 20° C. for 12 h under nitrogen atmosphere. On completion, the mixture was cooled in an ice bath and filtered. The filter cake was dried under reduced pressure. The crude product was triturated with Petroleum ether/Ethanol=10/1 at 15° C. for 2 h, then filtered to afford the title compound (1.1 g, 96% yield) as a white solid. LC-MS (ESI⁺) m/z 320.1 (M+H); ¹H NMR (400 MHz, DMSO-d₆) δ=7.99 (d, J=6.4 Hz, 1H), 7.69 (s, 1H), 3.25 (br s, 3H), 3.04 (br s, 3H).

### 5-(1-(3-(1H-pyrazol-1-yl)propanoyl)-1,2,5,6-tetrahydropyridin-3-yl)-4-fluoro-7-(2-methoxy-4-(piperazin-1-yl)phenyl)-N,N-dimethylbenzofuran-2-carboxamide (Intermediate AZ)

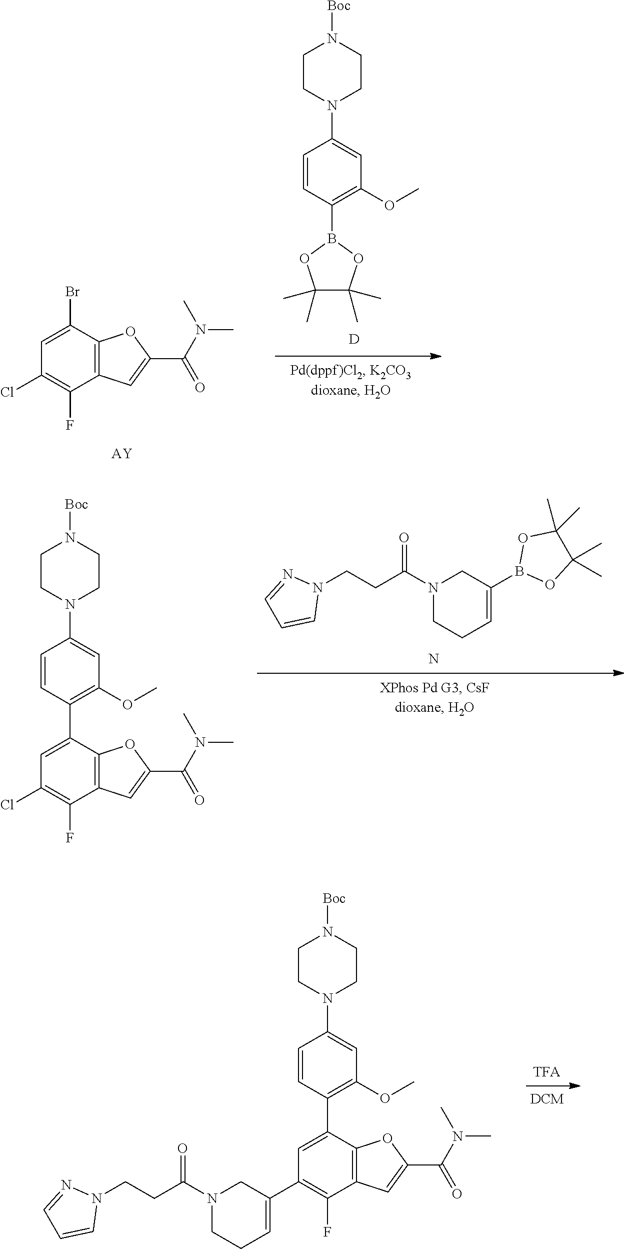

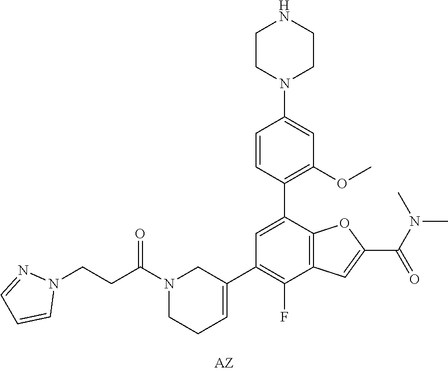 [SMILES: COc1cc(N2CCNCC2)ccc1-c1cc(C2=CCCN(C(=O)CCn3cccn3)C2)c(F)c2cc(C(=O)N(C)C)oc12]

### Step 1—Tert-butyl 4-(4-(5-chloro-2-(dimethylcarbamoyl)-4-fluorobenzofuran-7-yl)-3-methoxyphenyl)piperazine-1-carboxylate

A mixture of 7-bromo-5-chloro-4-fluoro-N,N-dimethyl-benzofuran-2-carboxamide (600 mg, 1.87 mmol. Intermediate AY), tert-butyl 4-[3-methoxy-4-(4,4,5,5-tetramethyl-1,3,2-dioxaborolan-2-yl)phenyl]piperazine-1-carboxylate (783 mg, 1.87 mmol, Intermediate D), Pd(dppf)Cl₂ (136 mg, 187 gmol), and K₂CO₃ (641 mg, 4.68 mmol) in dioxane (4 mL), H₂O (1 mL) was degassed and purged with N₂ three times. Then the mixture was stirred at 80° C. for 2 h under N₂ atmosphere. On completion, the reaction mixture was poured into H₂O (10 mL) and extracted with EtOAc (10 mL×3). The combined organic layers were washed with brine (20 mL×2), dried over Na₂SO₄ filtered and concentrated under reduced pressure to give a residue. The residue was purified by column chromatography (SiO₂, Petroleum ether:Ethyl acetate 5:1 to 0:1) to give the title compound (660 mg, 66% yield) as a white solid. LC-MS (ESI⁺) m/z 490.2 (M-55)⁺.

### Step 2—Tert-butyl 4-(4-(5-(1-(3-(1H-pyrazol-1-yl)propanoyl)-1,2,5,6-tetrahydropyridin-3-yl)-2-(dimethylcarbamoyl)-4-fluorobenzofuran-7-yl)-3-methoxyphenyl)piperazine-1-carboxylate

A mixture of tert-butyl 4-[4-[5-chloro-2-(dimethylcarbamoyl)-4-fluoro-benzofuran-7-yl]-3-methoxy-phenyl]piperazine-1-carboxylate (660 mg, 1.24 mmol), 3-pyrazol-1-yl-1-[5-(4,4,5,5-tetramethyl-1,3,2-dioxaborolan-2-yl)-3,6-dihydro-2H-pyridin-1-yl]propan-1-one (410 mg, 1.24 mmol, Intermediate N), XPhos Pd G3 (105 mg, 124 μmol), and CsF (565 mg, 3.72 mmol, 137 μL) in dioxane (6 mL) and H₂O (1 mL) was degassed and purged with N₂ three times. Then the mixture was stirred at 80° C. for 2 h under N₂ atmosphere. On completion, the reaction mixture was poured into H₂O (10 mL) and extracted with EtOAc (10 mL×3). The combined organic layers were washed with brine (20 mL×2), dried over Na₂SO₄ filtered and concentrated under reduced pressure to give a residue The residue was purified by column chromatography (SiO₂, Petroleum ether:Ethyl acetate=5:1 to 0:1) to give the title compound (600 mg, 69% yield) as a white solid. LC-MS (ESI⁺) m/z 701.4 (M+H)⁺.

### Step 3—5-(1-(3-(1H-pyrazol-1-yl)propanoyl)-1,2,5,6-tetrahydropyridin-3-yl)-4-fluoro-7-(2-methoxy-4-(piperazin-1-yl)phenyl)-N,N-dimethylbenzofuran-2-carboxamide

To a solution of tert-butyl 4-[4-[2-(dimethylcarbamoyl)-4-fluoro-5-[I-(3-pyrazol-1-ylpropanoyl)-3,6-dihydro-2H-pyridin-5-yl]benzofuran-7-yl]-3-methoxy-phenyl]piperazine-1-carboxylate (200 mg, 285 gmol) in DCM (1 mL) was added TFA (0.2 mL). The mixture was then stirred at 25° C. for 1 h. On completion, the mixture was concentrated under reduced pressure to give the title compound (150 mg, TFA) as a white solid. LC-MS (ESI⁺) m/z 601.3 (M+H)⁺.

### 5-(1-(3-(1H-pyrazol-1-yl)propanoyl)-1,2,5,6-tetrahydropyridin-3-yl)-7-(2-(difluoromethoxy)-4-(piperazin-1-yl)phenyl)-4-fluoro-N,N-dimethylbenzofuran-2-carboxamide (Intermediate BA)

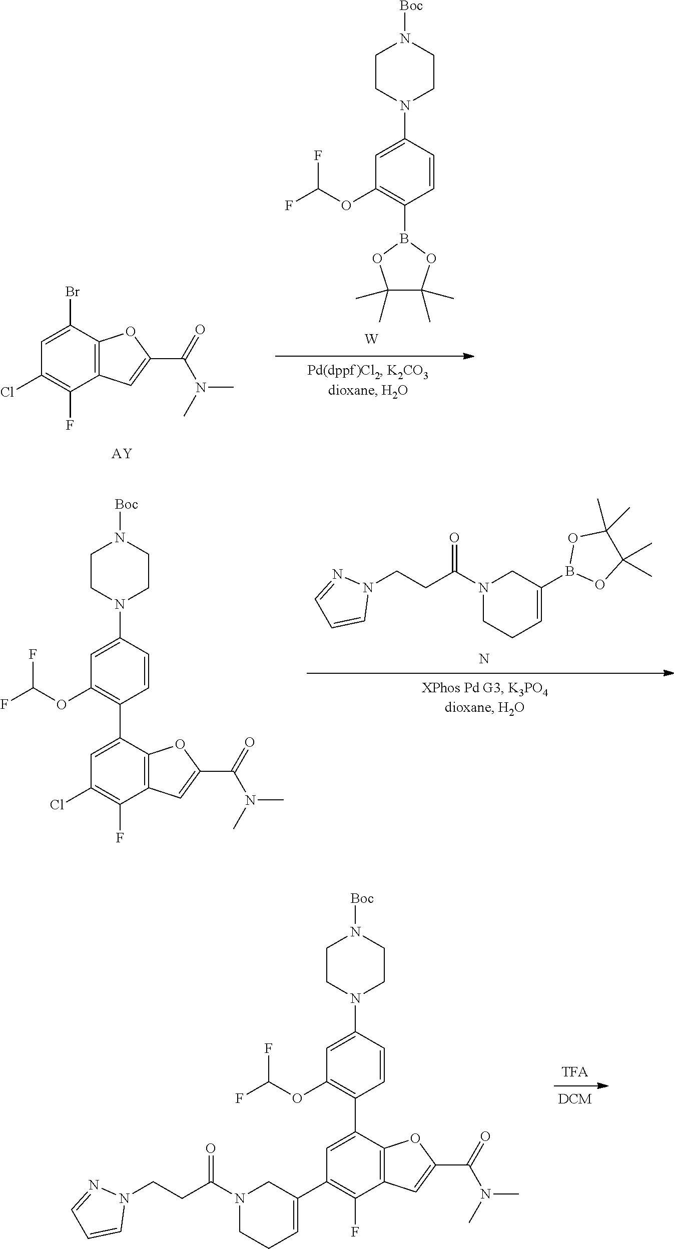

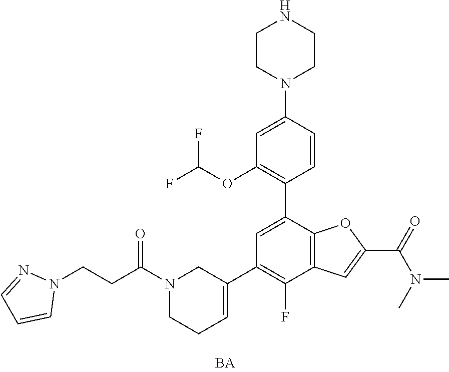 [SMILES: CN(C)C(=O)c1cc2c(F)c(C3=CCCN(C(=O)CCn4cccn4)C3)cc(-c3ccc(N4CCNCC4)cc3OC(F)F)c2o1]

### Step 1—Tert-butyl 4-(4-(5-chloro-2-(dimethylcarbamoyl)-4-fluorobenzofuran-7-yl)-3-(difluoromethoxy)phenyl)piperazine-1-carboxylate

To a solution of 7-bromo-5-chloro-4-fluoro-N,N-dimethylbenzofuran-2-carboxamide (1 g, 3.12 mmol, Intermediate AY) and tert-butyl 4-[3-(difluoromethoxy)-4-(4,4,5,5-tetramethyl-1,3,2-dioxaborolan-2-yl)phenyl]piperazine-1-carboxylate (1.56 g, 3.43 mmol, Intermediate W) in dioxane (10 mL) and H₂O (2 mL) was added Pd(dppf)Cl₂ (228 mg, 312 μmol) and K₂CO₃ (1.29 g, 9.36 mmol) at 25° C. Then the mixture was stirred at 80° C. for 2 hrs. On completion, the reaction mixture was concentrated in vacuo to give the crude residue. The residue was purified by column chromatography (SiO₂, Petroleum ether/Ethyl acetate=2/1 to 1/1) to give the title compound (1.35 g, 64% yield) as a yellow solid. LC-MS (ESI⁺) m/z 512.1 (M-55)⁺.

### Step 2—Tert-butyl 4-(4-(5-(1-(3-(1H-pyrazol-1-yl)propanoyl)-1,2,5,6-tetrahydropyridin-3-yl)-2-(dimethylcarbamoyl)-4-fluorobenzofuran-7-yl)-3-(difluoromethoxy)phenyl)piperazine-1-carboxylate

To a solution of tert-butyl 4-(4-(5-chloro-2-(dimethylcarbamoyl)-4-fluorobenzofuran-7-yl)-3-(difluoromethoxy)phenyl)piperazine-1-carboxylate (600 mg, 1.06 mmol) and 3-(1H-pyrazol-1-yl)-1-(5-(4,4,5,5-tetramethyl-1,3,2-dioxaborolan-2-yl)-3,6-dihydropyridin-1(2H)-yl)propan-1-one (525 mg, 1.58 mmol. Intermediate N) in dioxane (0.5 mL) and H₂O (0.1 mL) was added XPhos Pd G3 (89.4 mg, 106 mol) and K₃PO₄ (673 mg, 3.17 mmol) at 25° C. Then the mixture was stirred at 80° C. for 2 hrs. On completion, the reaction mixture was concentrated in vacuo to give the crude residue. The residue was purified by column chromatography (SiO₂, DCM:MeOH=50:1) to give the title compound (550 mg, 61% yield) as a yellow solid. LC-MS (ESI⁺) m/z 737.3 (M+H)⁺.

### Step 3—5-(1-(3-(1H-pyrazol-1-yl)propanoyl)-1,2,5,6-tetrahydropyridin-3-yl)-7-(2-(difluoromethoxy)-4-(piperazin-1-yl)phenyl)-4-fluoro-N,N-dimethylbenzofuran-2-carboxamide

To a solution of tert-butyl 4-(4-(5-(1-(3-(1H-pyrazol-1-yl)propanoyl)-1,2,5,6-tetrahydropyridin-3-yl)-2-(dimethylcarbamoyl)-4-fluorobenzofuran-7-yl)-3-(difluoromethoxy)phenyl)piperazine-1-carboxylate (120 mg, 1623 μmol) in DCM (1 mL) was added TFA (553 mg, 4.85 mmol, 360 μL) at 25° C., then the mixture was stirred at 25° C. for 1 hr. On completion, the reaction mixture was concentrated in vacuo to give the crude residue. The crude product was purified by reverse phase HPLC (0.8 g/L NH₄HCO₃) to give the title compound (100 mg, 96% yield) as a white solid. LC-MS (ESI⁺) m/z 637.2 (M+H)⁺.

### 6-(1-(3-(1H-1,2,3-triazol-1-yl)propanoyl)-1,2,5,6-tetrahydropyridin-3-yl)-7-fluoro-4-(2-methoxy-4-(piperazin-1-yl)phenyl)-N,N-dimethyl-1H-indole-2-carboxamide (Intermediate BB)

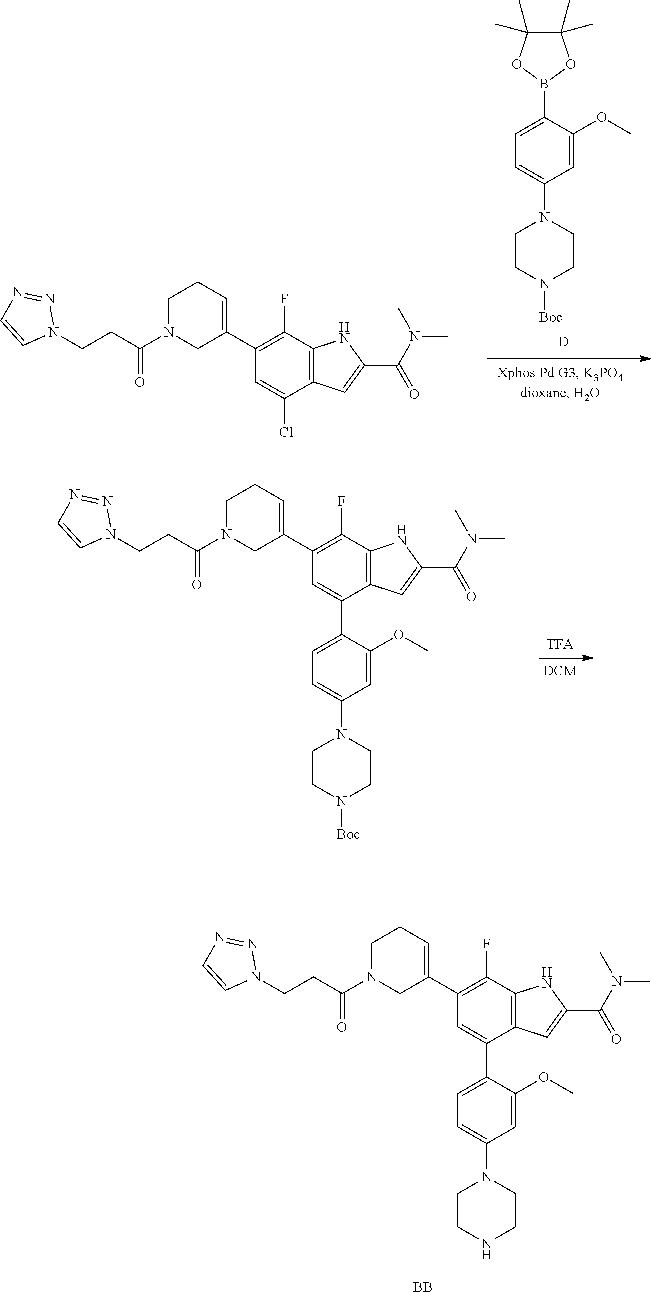

### Step 1—Tert-butyl 4-(4-(6-(1-(3-(1H-1,2,3-triazol-1-yl)propanoyl)-1,2,5,6-tetrahydropyridin-3-yl)-2-(dimethylcarbamoyl)-7-fluoro-1H-indol-4-yl)-3-methoxyphenyl)piperazine-1-carboxylate

A solution of 6-(1-(3-(1H-1,2,3-triazol-1-yl)propanoyl)-1,2,5,6-tetrahydropyridin-3-yl)-4-chloro-7-fluoro-N,N-dimethyl-1H-indole-2-carboxamide (400 mg, 62 gmol, synthesized via Step 1 of Intermediate AK), tert-butyl 4-(3-methoxy-4-(4,4,5,5-tetramethyl-1,3,2-dioxaborolan-2-yl)phenyl)piperazine-1-carboxylate (289 mg, 692 μmol, Intermediate D), XPhos Pd G3 (53.2 mg, 62.9 μmol, CAS #1445085-55-1), and K₃PO₄ (400 mg, 1.89 mmol) in dioxane (2 mL) and H₂O (0.5 mL) was degassed and purged with N₂ three times. Then the mixture was stirred at 80° C. for 2 hrs under N₂ atmosphere. On completion, the reaction mixture was concentrated in vacuo to give the title compound (400 mg) as a black solid. LC-MS (ESI⁺) m/z 701.3 (M+1)⁺.

### Step 2—6-(1-(3-(1H-1,2,3-triazol-1-yl)propanoyl)-1,2,5,6-tetrahydropyridin-3-yl)-7-fluoro-4-(2-methoxy-4-(piperazin-1-yl)phenyl)-N,N-dimethyl-1H-indole-2-carboxamide

To a solution of tert-butyl 4-(4-(6-(1-(3-(TH-1,2,3-triazol-1-yl)propanoyl)-1,2,5,6-tetrahydropyridin-3-yl)-2-(dimethylcarbamoyl)-7-fluoro-1H-indol-4-yl)-3-methoxyphenyl)piperazine-1-carboxylate (200 mg, 285 μmol) in DCM (3 mL) was added TFA (32.5 mg, 285 μmol, 21.2 IL). The mixture was then stirred at 25° C. for 2 hrs. On completion, the reaction mixture was concentrated in vacuo to give the title compound (170 mg, TFA) as a white solid. LC-MS (ESI⁺) m/z 601.5 (M+1)⁺.

### 6-Bromo-4-chloro-7-fluoro-1H-indole (Intermediate BC)

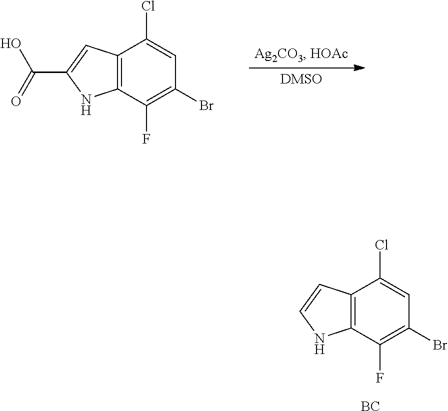

A mixture of 6-bromo-4-chloro-7-fluoro-1H-indole-2-carboxylic acid (1.5 g, 5.13 mmol, synthesized via Step 1 of Intermediate T), Ag₂CO₃ (424 mg, 1.54 mmol), and HOAc (923 mg, 15.4 mmol) in DMSO (5 mL) was degassed and purged with N₂ three times. Then the mixture was stirred at 120° C. for 12 hrs under N₂ atmosphere. On completion, the reaction mixture was quenched by addition of NH₄Cl (50 mL) at 25° C., and then extracted with ethyl acetate (3×50 mL). The combined organic layers were dried over anhydrous sodium sulfate, filtered and the filtrate was concentrated in vacuo to give the crude residue. The residue was purified by column chromatography (SiO₂. Petroleum ether/Ethyl acetate=15/1 to 8/1) to give the title compound (350 mg, 17% yield) as a brown solid. LC-MS (ESI⁺) m/z 247.6 (M+H)⁺.

### 1-(5-(7-fluoro-4-(4,4,5,5-tetramethyl-1,3,2-dioxaborolan-2-yl)-1H-indol-6-yl)-3,6-dihydropyridin-1(2H)-yl)-3-(1H-1,2,3-triazol-1-yl)propan-1-one (Intermediate BD)

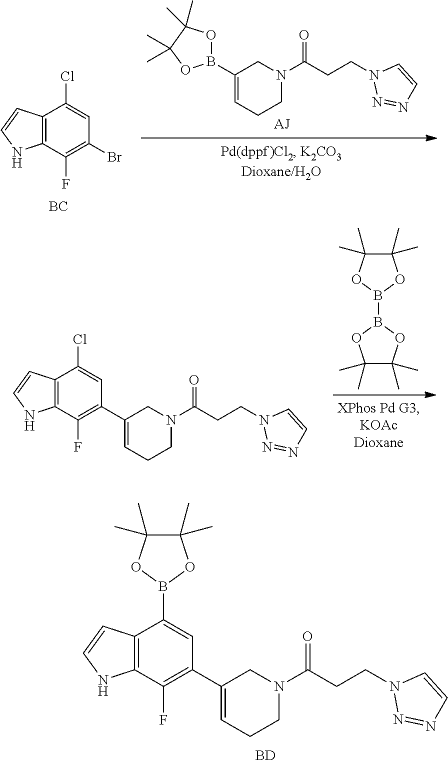

### Step 1—1-(5-(4-Chloro-7-fluoro-1H-indol-6-yl)-3,6-dihydropyridin-1(2H)-yl)-3-(1H-1,2,3-triazol-1-yl)propan-1-one

A mixture of 6-bromo-4-chloro-7-fluoro-1H-indole (300 mg, 1.21 mmol, Intermediate BC), 1-(5-(4,4,5,5-tetramethyl-1,3,2-dioxaborolan-2-yl)-3,6-dihydropyridin-1(2H)-yl)-3-(1H-1,2,3-triazol-1-yl)propan-1-one (601 mg, 1.81 mmol, Intermediate AJ), K₂CO₃ (500 mg, 3.62 mmol), and Pd(dppf)Cl₂·CH₂Cl₂ (98.6 mg, 120 μmol) in dioxane (10 mL), H₂O (2 mL) was degassed and purged with N₂ three times. Then the mixture was stirred at 80° C. for 2 hrs under N₂ atmosphere. On completion, the reaction mixture was concentrated under reduced pressure to remove solvent and to give a residue. The residue was purified by column chromatography (SiO₂, Petroleum ether/Ethyl acetate=10/1 to 0/1, DCM:MeOH=20:1) to give the title compound (400 mg, 48% yield) as a brown solid. LC-MS (ESI⁺) m/z 371.8 (M+H)⁺.

### Step 2—1-(5-(7-Fluoro-4-(4,4,5,5-tetramethyl-1,3,2-dioxaborolan-2-yl)-1H-indol-6-yl)-3,6-dihydropyridin-1(2H)-yl)-3-(1H-1,2,3-triazol-1-yl)propan-1-one

A mixture of 1-(5-(4-chloro-7-fluoro-1H-indol-6-yl)-3,6-dihydropyridin-1(2H)-yl)-3-(1H-1,2,3-triazol-1-yl)propan-1-one (370 mg, 989 μmol), 4,4,4′,4′,5,5,5′,5′-octamethyl-2,2′-bi(1,3,2-dioxaborolane) (502 mg, 1.98 mmol, CAS #73183-34-3), KOAc (291 mg, 2.97 mmol), XPhos Pd G3 (167 mg, 197 gmol) and in dioxane (5 mL) was degassed and purged with N₂ three times. Then the mixture was stirred at 80° C. for 2 hrs under N₂ atmosphere. On completion, the reaction mixture was concentrated under reduced pressure to remove solvent to give a residue. The residue was purified by column chromatography (SiO₂, Petroleum ether/Ethyl acetate=5/1 to 0/1, DCM:MeOH=20:1) to give the title compound (300 mg, 55% yield) as a white solid. LC-MS (ESI⁺) m/z 466.0 (M+H)⁺.

### 7-Fluoro-6-(1-isobutyryl-1,2,5,6-tetrahydropyridin-3-yl)-N,N-dimethyl-4-(4-(piperazin-1-yl)-2-(trifluoromethoxy)phenyl)-1H-indole-2-carboxamide (Intermediate BE)

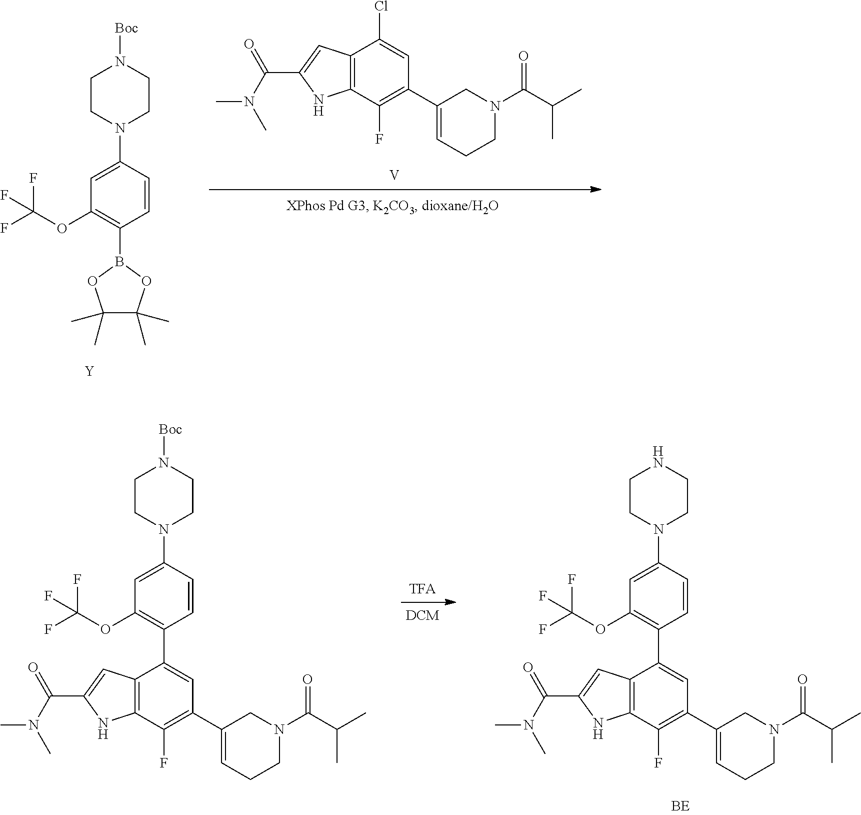

### Step 1—Tert-butyl 4-(4-(2-(dimethylcarbamoyl)-7-fluoro-6-(1-isobutyryl-1,2,5,6-tetrahydropyridin-3-yl)-1H-indol-4-yl)-3-(trifluoromethoxy)phenyl)piperazine-1-carboxylate

A mixture of 4-chloro-7-fluoro-N,N-dimethyl-6-[1-(2-methylpropanoyl)-3,6-dihydro-2H-pyridin-5-yl]-1H-indole-2-carboxamide (110 mg, 280 μmol, Intermediate V), tert-butyl 4-[4-(4,4,5,5-tetramethyl-1,3,2-dioxaborolan-2-yl)-3-(trifluoromethoxy)phenyl]piperazine-1-carboxylate (132 mg, 280 mol, Intermediate Y), XPhos Pd G3 (24.0 mg, 28.0 μmol), K₃PO₄ (178 mg, 842 mol) in dioxane (2 mL) and H₂O (0.4 mL) was degassed and purged with N₂ three times. Then the mixture was stirred at 80° C. for 1 h under N₂ atmosphere. The reaction mixture was diluted with H₂O (20 mL) and extracted with ethyl acetate (20 mL×3). The combined organic layers were dried over Na₂SO₄, filtered and concentrated under reduced pressure to give a residue. The residue was purified by flash silica gel chromatography (ISCO®4 g SepaFlash® Silica Flash Column, Eluent of 0˜50% Ethyl acetate/Petroleum ether gradient @30 mL/min) to afford the title compound (150 mg, 72% yield) as a yellow solid. LC-MS (ESI⁺) m/z 702.2 (M+H)⁻; ¹H NMR (400 MHz, CDCl₃) δ ppm 9.48-9.36 (m, 1H), 7.44 (d, J=6.4 Hz, 1H), 7.08-6.89 (m, 3H), 6.73-6.55 (m, 1H), 6.16 (s, 1H), 4.57-4.36 (m, 2H), 3.88-3.53 (m, 6H), 3.47-3.03 (m, 10H), 2.98-2.77 (m, 1H), 2.49-2.28 (m, 2H), 1.55 (d, J=2.0 Hz, 8H), 1.17 (dd. J=7.2, 10.4 Hz, 6H).

### Step 2—7-Fluoro-6-(1-isobutyryl-1,2,5,6-tetrahydropyridin-3-yl)-N,N-dimethyl-4-(4-(piperazin-1-yl)-2-(trifluoromethoxy)phenyl)-1H-indole-2-carboxamide

To a solution of tert-butyl 4-[4-[2-(dimethylcarbamoyl)-7-fluoro-6-[1-(2-methylpropanoyl)-3,6-dihydro-2H-pyridin-5-yl]-1H-indol-4-yl]-3-(trifluoromethoxy)phenyl]piperazine-1-carboxylate (140 mg, 199 μmol) in DCM (1.2 mL) was added TFA (414 L g, 5.59 mmol). The mixture was then stirred at 25° C. for 1 h. On completion, the reaction mixture was concentrated under reduced pressure to afford the title compound (279 mg, TFA) as yellow oil. LC-MS (ESI⁺) m/z 602.3 (M+H)⁺.

### 3-((4-(4-(4-Chlorophenyl)piperidin-1-yl)-5-fluoro-2-methoxyphenyl)amino)piperidine-2,6-dione (Intermediate BF)

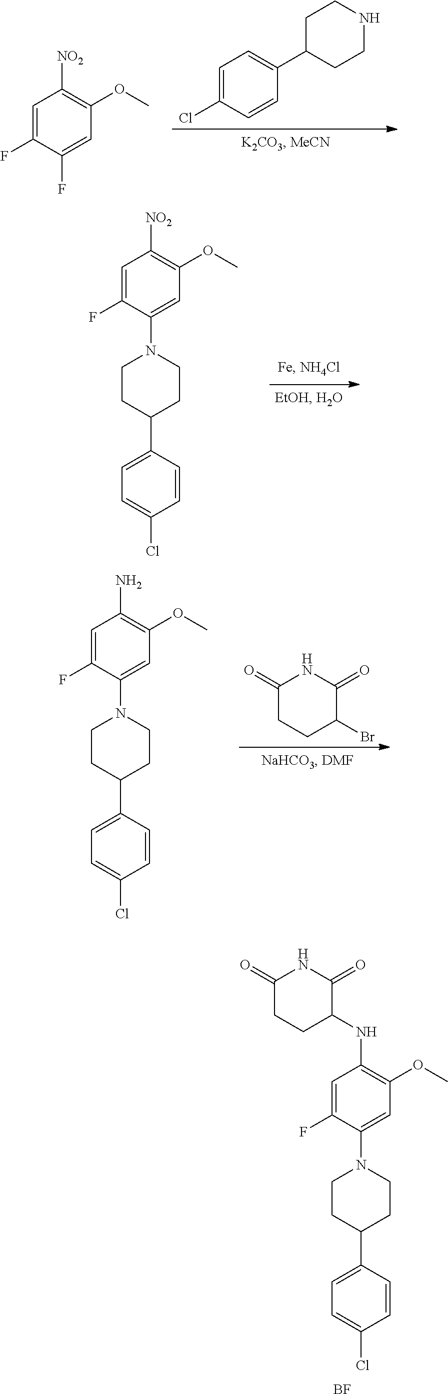

### Step 1—4-(4-Chlorophenyl)-1-(2-fluoro-5-methoxy-4-nitrophenyl)piperidine

To a solution of 1,2-difluoro-4-methoxy-5-nitro-benzene (1.60 g, 8.46 mmol, CAS #66684-64-8) in ACN (20 mL) was added 4-(4-chlorophenyl)piperidine (1.99 g, 10.2 mmol. CAS #26905-02-2) and K₂CO₃ (3.51 g, 25.4 mmol). The mixture was then stirred at 80° C. for 4 hrs. On completion, the reaction mixture was diluted with H₂O (90 mL) and extracted with ethyl acetate (30 mL×3). The combined organic layers were washed with brine (30 mL×3), dried over sodium sulfate, filtered and concentrated under reduced pressure to give a residue. The residue was purified by column chromatography (SiO₂, Petroleum ether/Ethyl acetate=6/1 to 0/1) to give the title compound (3 g, 97% yield) as a white solid. LC-MS (ESI⁺) m/z 365.0 (M+H)⁺.

### Step 2—4-(4-(4-Chlorophenyl)piperidin-1-yl)-5-fluoro-2-methoxyaniline

To a solution of 4-(4-chlorophenyl)-1-(2-fluoro-5-methoxy-4-nitro-phenyl)piperidine (500 mg, 1.37 mmol) in EtOH (4 mL) and H₂O (2 mL) was added Fe (765 mg, 13.7 mmol) and NH₄Cl (733 mg, 13.7 mmol). The mixture was then stirred at 80° C. for 6 hrs. On completion, the reaction mixture was filtered and concentrated in vacuo to give the title compound (440 mg) as a white solid. LC-MS (ESI⁺) m/z 334.9 (M+H)⁺.

### Step 3—3-((4-(4-(4-Chlorophenyl)piperidin-1-yl)-5-fluoro-2-methoxyphenyl)amino)piperidine-2,6-dione

To a solution of 4-[4-(4-chlorophenyl)-1-piperidyl]-5-fluoro-2-methoxy-aniline (440 mg, 1.31 mmol) in DMF (5 mL) was added NaHCO₃ (221 mg, 2.63 mmol) and 3-bromopiperidine-2,6-dione (631 mg, 3.29 mmol, CAS #62595-74-8). The mixture was then stirred at 80° C. for 4 hrs. On completion, the reaction mixture was filtered and concentrated in vacuo to give the crude residue. The crude residue was purified by reverse phase HPLC (0.1% FA condition) to give the title compound (250 mg, 41% yield) as a white solid. LC-MS (ESI⁺) m/z 446.0 (M+H)⁺.

### 3-((4-((4-Chlorophenyl)ethynyl)-3-fluorophenyl)amino)piperidine-2,6-dione (Intermediate BG)

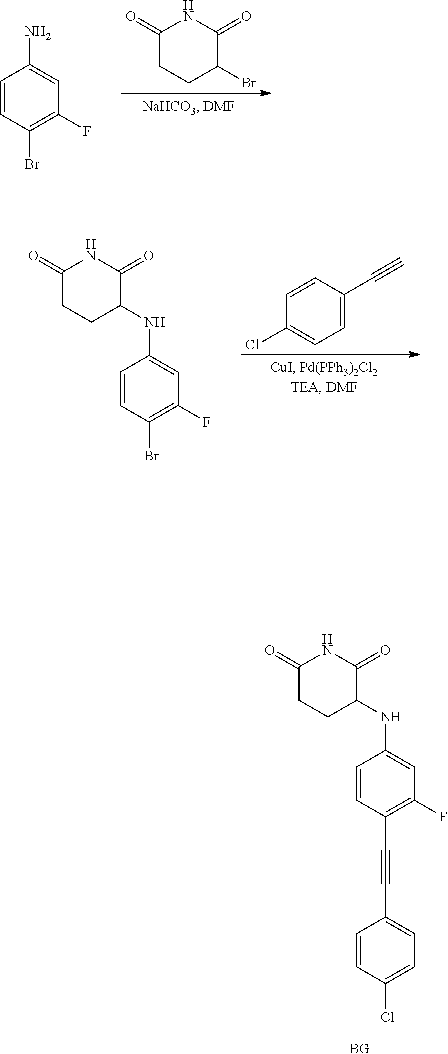

### Step 1—3-((4-Bromo-3-fluorophenyl)amino)piperidine-2,6-dione

To a solution of 4-bromo-3-fluoro-aniline (20 g, 105 mmol. CAS #656-65-5) and 3-bromopiperidine-2,6-dione (40.4 g, 210 mmol, CAS #62595-74-8) in DMF (200 mL) was added NaHCO₃ (17.7 g, 210 mmol, 8.19 mL) at 25° C., then the mixture was stirred at 80° C. for 3 h. On completion, the reaction mixture was filtered and the filtrate was quenched by addition of H₂O (800 mL) at 25° C., and then diluted with ethyl acetate (500 mL) and extracted with ethyl acetate (500 mL×2). The combined organic layers were dried over Na₂SO₄, filtered and concentrated under reduced pressure to give a residue. The residue was purified by column chromatography (SiO₂, Petroleum ether/Ethyl acetate=5/1 to 0/1) to give the title compound (9.5 g, 29% yield) as a blue solid. LC-MS (ESI⁺) m/z 300.8 (M+H)⁺.

### Step 2—3-((4-((4-Chlorophenyl)ethynyl)-3-fluorophenyl)amino)piperidine-2,6-dione

A mixture of 3-(4-bromo-3-fluoro-anilino)piperidine-2,6-dione (600 mg, 1.39 mmol), 1-chloro-4-ethynyl-benzene (191 mg, 1.39 mmol, CAS #873-73-4), CuI (13.3 mg, 69.7 μmol), TEA (565 mg, 5.58 mmol, 777 μL) and Pd(PPh₃)₂Cl₂ (49.0 mg, 69.7 μmol) in DMF (6 mL) was degassed and purged with N₂ three times. Then the mixture was stirred at 80° C. for 2 hrs under N₂ atmosphere. On completion, the reaction mixture was filtered and concentrated under reduced pressure to give a residue. The crude product was purified by reverse phase HPLC (0.1% FA condition) to give the title compound (150 mg, 25% yield) as a white solid. LC-MS (ESI⁺) m/z 357.1 (M+H)⁺.

### 4-(4-Chloro-3-(trifluoromethyl)phenyl)piperidine (Intermediate BH)

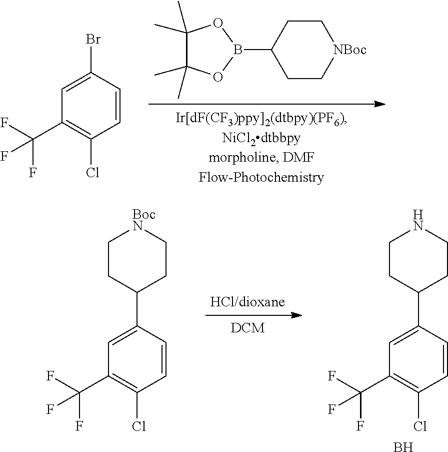

### Step 1—Tert-butyl 4-(4-chloro-3-(trifluoromethyl)phenyl)piperidine-1-carboxylate

To a 500 mL vial equipped with a stir bar was added 4-bromo-1-chloro-2-(trifluoromethyl)benzene (6 g, 23.1 mmol, 3.44 mL), tert-butyl 4-(4,4,5,5-tetramethyl-1,3,2-dioxaborolan-2-yl)piperidine-1-carboxylate (7.20 g, 23.1 mmol), Ir[dF(CF₃)ppy]₂(dtbpy)(PF₆) (259 mg, 231 μmol), NiCl₂·dtbbpy (460 mg, 1.16 mmol) and morpholine (3.02 g, 34.7 mmol, 3.05 mL) in DMF (240 mL). The reaction solution was pumped through the reactor at a flow rate of 0.3 mL/min, irradiating with a 450 min LED lamp, with cooling fan to keep the reaction temperature at 25° C. for 14 h. On completion, the reaction mixture was diluted with H₂O (500 mL) and extracted with ethyl acetate (200 mL×3). The combined organic layers were washed with brine (200 mL×2), dried over Na₂SO₄, filtered and concentrated under reduced pressure to give a residue. The crude residue was purified by reverse phase HPLC (0.1% FA condition) to give the title compound (4 g, 75% yield) as a brown oil. LC-MS (ESI⁺) m/z 307.9 (M-55)⁺.

### Step 2—4-(4-Chloro-3-(trifluoromethyl)phenyl)piperidine

A mixture of tert-butyl 4-(4-chloro-3-(trifluoromethyl)phenyl)piperidine-1-carboxylate (2.2 g, 6.05 mmol) in DCM (10 mL) was added HCl/dioxane (2.5 mL, 4 M). The mixture was then stirred at 25° C. for 2 hrs. On completion, the mixture was filtered and concentrated under reduced pressure to give the title compound (2 g) as a yellow oil. LC-MS (ESI⁺) m/z 264.0 (M+H)⁺.

### 3-(4-Bromo-3-methyl-2-oxo-2,3-dihydro-1H-benzo[d]imidazol-1-yl)-1-(4-methoxybenzyl)piperidine-2,6-dione (Intermediate BI)

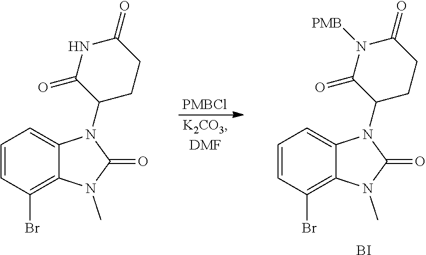

To a solution of 3-(4-bromo-3-methyl-2-oxo-2,3-dihydro-1H-benzo[d]imidazol-1-yl)piperidine-2,6-dione (20 g, 59.1 mmol, Intermediate H) and PMB-Cl (10.2 g, 65.0 mmol, 8.83 mL) in DMF (400 mL) was added K₂CO₃ (24.5 g, 177 mmol). The mixture was then stirred at 40° C. for 12 h. On completion, the reaction mixture was filtered and diluted with H₂O (2000 mL) and extracted with EtOAc (300 mL×3). The combined organic layers were washed with brine (600 mL×2), dried over Na₂SO₄, filtered and concentrated under reduced pressure to give a residue. The crude product was triturated with Petroleum ether:Ethyl acetate=10/1(500 mL) at 25° C. for 30 min to give the title compound (26 g, 85% yield) as a gray solid. LC-MS (ESI⁺) m/z 458.1 (M+H)⁺.

### 3-(4-(4-(4-Chloro-3-(trifluoromethyl)phenyl)piperidin-1-yl)-3-methyl-2-oxo-2,3-dihydro-1H-benzo[d]imidazol-1-yl)piperidine-2,6-dione (Intermediate BJ)

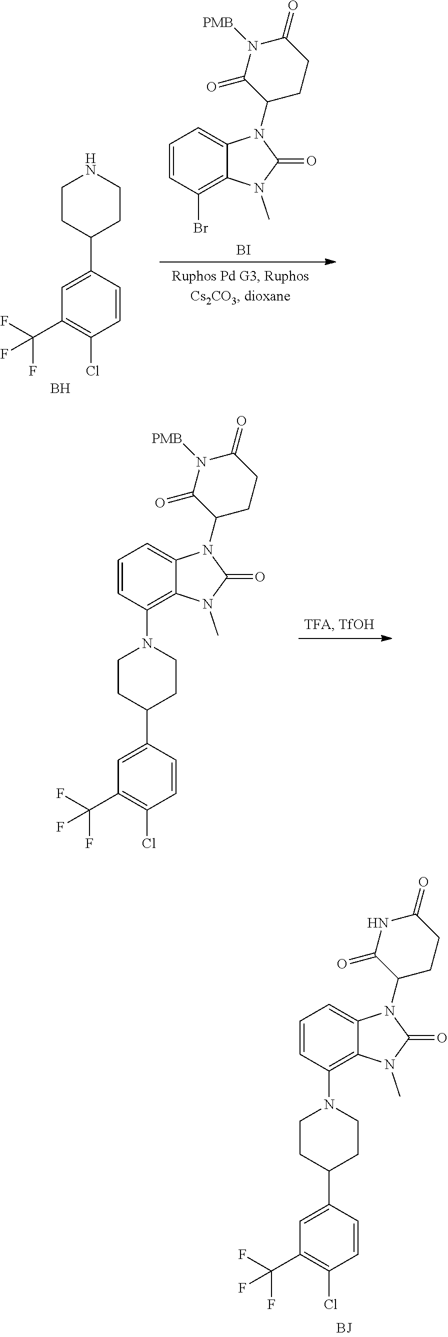

### Step 1—3-(4-(4-(4-Chloro-3-(trifluoromethyl)phenyl)piperidin-1-yl)-3-methyl-2-oxo-2,3-dihydro-1H-benzo[d]imidazol-1-yl)-1-(4-methoxybenzyl)piperidine-2,6-dione

To a solution of 4-(4-chloro-3-(trifluoromethyl)phenyl)piperidine (690 mg, 2.62 mmol, Intermediate BH), 3-(4-bromo-3-methyl-2-oxo-2,3-dihydro-1H-benzo[d]imidazol-1-yl)-1-(4-methoxybenzyl)piperidine-2,6-dione (1 g, 2 mmol, Intermediate BI), Ruphos Pd G3 (547 mg, 655 μmol), Ruphos (305 mg, 655 μmol), and Cs₂CO₃ (3.55 g, 10.9 mmol) in dioxane (8 mL). The mixture was stirred at 100° C. for 2 hrs. On completion, the mixture was diluted with water (50 mL) and extracted with dichloromethane (50 mL×2), dried over sodium sulfate, filtered and concentrated under reduced pressure. The residue was purified by prep-HPLC (FA condition) to give the title compound (60 mg, 18% yield) as a yellow oil. LC-MS (ESI⁻) m/z 641.1 (M+H)⁺.

### Step 2—3-(4-(4-(4-Chloro-3-(trifluoromethyl)phenyl)piperidin-1-yl)-3-methyl-2-oxo-2,3-dihydro-1H-benzo[d]imidazol-1-yl)piperidine-2,6-dione

To a solution of 3-(4-(4-(4-chloro-3-(trifluoromethyl)phenyl)piperidin-1-yl)-3-methyl-2-oxo-2,3-dihydro-1H-benzo[d]imidazol-1-yl)-1-(4-methoxybenzyl)piperidine-2,6-dione (230 mg, 359 gmol) in TFA (3 mL) was added TfOH (1 mL), then the mixture was stirred at 60° C. for 12 hrs. On completion, the mixture was filtered and concentrated under reduced pressure, purified by prep-HPLC (FA condition) to give the title compound (103 mg, 50% yield, FA) as a white solid. LC-MS (ESI⁺) m/z 521. (M+H)⁺.

### 7-Fluoro-6-(1-isobutyryl-1,2,5,6-tetrahydropyridin-3-yl)-N,N-dimethyl-4-(4,4,5,5-tetramethyl-1,3,2-dioxaborolan-2-yl)-1H-indole-2-carboxamide (Intermediate BK)

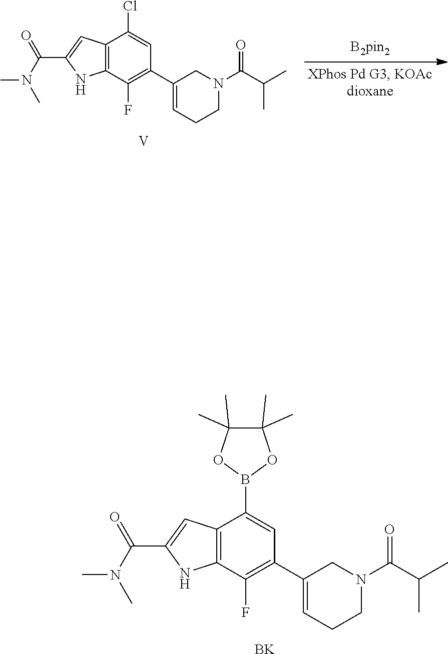

To a solution of 4,4,5,5-tetramethyl-2-(4,4,5,5-tetramethyl-1,3,2-dioxaborolan-2-yl)-1,3,2-dioxaborolane (5.67 g, 22.33 mmol) in dioxane (100 mL) was added 4-chloro-7-fluoro-6-(1-isobutyryl-1,2,5,6-tetrahydropyridin-3-yl)-N,N-dimethyl-1H-indole-2-carboxamide (3.5 g, 8.93 mmol, Intermediate V), AcOK (2.63 g, 26.8 mmol) and XPhos Pd G3 (756 mg, 893 μmol). The reaction was then stirred at 80° C. for 2 hrs. On completion, the reaction mixture was concentrated in vacuo to give the crude residue. The residue was purified by column chromatography (SiO₂, Petroleum ether:Ethyl acetate=20/1 to 5/1) to give the title compound (5.1 g, 98% yield) as a brown solid. LC-MS (ESI⁺) m/z 484.2 (M+H)⁺.

### Tert-butyl (R)-4-(4-(5-(1-(3-(1H-pyrazol-1-yl)propanoyl)piperidin-3-yl)-2-(dimethylcarbamoyl)-4-fluorobenzofuran-7-yl)phenyl)piperazine-1-carboxylate (Intermediate BL) and tert-butyl (S)-4-(4-(5-(1-(3-(1H-pyrazol-1-yl)propanoyl)piperidin-3-yl)-2-(dimethylcarbamoyl)-4-fluorobenzofuran-7-yl)phenyl)piperazine-1-carboxylate (Intermediate BM)

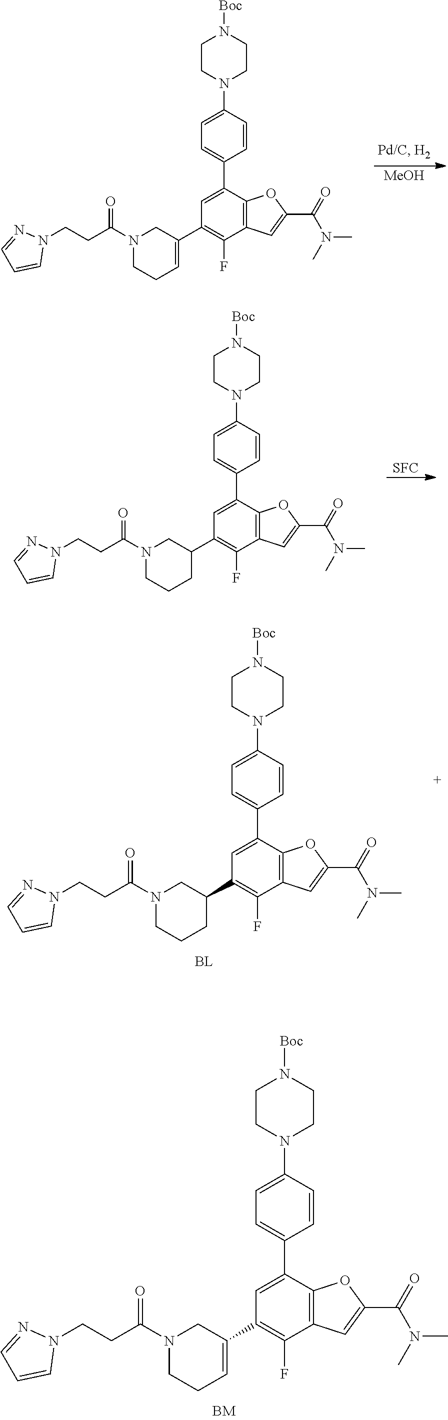

### Step 1—Tert-butyl 4-(4-(5-(1-(3-(1H-pyrazol-1-yl)propanoyl)piperidin-3-yl)-2-(dimethylcarbamoyl)-4-fluorobenzofuran-7-yl)phenyl)piperazine-1-carboxylate

To a solution of tert-butyl 4-(4-(5-(1-(3-(1H-pyrazol-1-yl)propanoyl)-1,2,5,6-tetrahydropyridin-3-yl)-2-(dimethylcarbamoyl)-4-fluorobenzofuran-7-yl)phenyl)piperazine-1-carboxylate (2.50 g, 3.73 mmol, synthesized via Step 1 of Intermediate O) in MeOH (45 mL) was added Pd/C (2.50 g, 2.35 mmol, 10 wt %). The mixture was then stirred at 40° C. for 12 hrs. On completion, the reaction mixture was filtered and the filtrate was concentrated in vacuo to give the title compound (2.2 g, 3.27 mmol) as a yellow solid. LC-MS (ESI⁺) m/z 673.3 (M+H)⁺.

### Step 2—Tert-butyl (R)-4-(4-(5-(1-(3-(1H-pyrazol-1-yl)propanoyl)piperidin-3-yl)-2-(dimethylcarbamoyl)-4-fluorobenzofuran-7-yl)phenyl)piperazine-1-carboxylate and tert-butyl (S)-4-(4-(5-(1-(3-(1H-pyrazol-1-yl)propanoyl)piperidin-3-yl)-2-(dimethylcarbamoyl)-4-fluorobenzofuran-7-yl)phenyl)piperazine-1-carboxylate

Tert-butyl 4-(4-(5-(1-(3-(1H-pyrazol-1-yl)propanoyl)piperidin-3-yl)-2-(dimethylcarbamoyl)-4-fluorobenzofuran-7-yl)phenyl)piperazine-1-carboxylate (2.2 g, 3.3 mmol) was separated by SFC (column: DAICEL CHIRALPAK AD(250 mm×50 mm, 10 um); mobile phase: [CO₂-ACN/i-PrOH(0.1% NH₃H₂O)]; B %: 50%, isocratic elution mode) to tert-butyl (R)-4-(4-(5-(1-(3-(1H-pyrazol-1-yl)propanoyl)piperidin-3-yl)-2-(dimethylcarbamoyl)-4-fluorobenzofuran-7-yl)phenyl)piperazine-1-carboxylate (1 g, 45% yield) and tert-butyl (S)-4-(4-(5-(1-(3-(1H-pyrazol-1-yl)propanoyl)piperidin-3-yl)-2-(dimethylcarbamoyl)-4-fluorobenzofuran-7-yl)phenyl)piperazine-1-carboxylate (0.9 g, 40.9% yield) as a white solids. LC-MS (ESI⁺) m/z 673.3 (M+H) for both isomers. The absolute stereochemistry of the enantiomers was arbitrarily assigned.

### (R)-5-(1-(3-(1H-pyrazol-1-yl)propanoyl)piperidin-3-yl)-4-fluoro-N,N-dimethyl-7-(4-(piperazin-1-yl)phenyl)benzofuran-2-carboxamide (Intermediate BN)

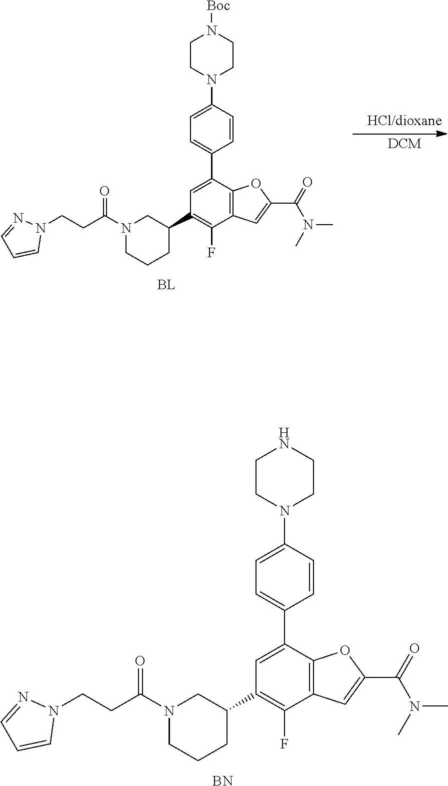

To a solution of tert-butyl (R)-4-(4-(5-(1-(3-(1H-pyrazol-1-yl)propanoyl)piperidin-3-yl)-2-(dimethylcarbamoyl)-4-fluorobenzofuran-7-yl)phenyl)piperazine-1-carboxylate (1 g, 1.49 mmol, Intermediate BL) in DCM (6 mL) was added HCl/dioxane (4 M, 2 mL). The mixture was then stirred at 25° C. for 1 hr. On completion, and the reaction mixture was concentrated under reduced pressure to give the title compound (0.8 g) as a yellow solid. LC-MS (ESI⁺) m/z 573.4 (M+H)⁻.

### (S)-6-(1-acetylpiperidin-3-yl)-7-fluoro-4-(2-methoxy-4-(piperazin-1-yl)phenyl)-N,N -dimethyl-1H-indole-2-carboxamide (Intermediate BO) and (R)-6-(1-acetylpiperidin-3-yl)-7-fluoro-4-(2-methoxy-4-(piperazin-1-yl)phenyl)-N,N-dimethyl-1H-indole-2-carboxamide (Intermediate BP)

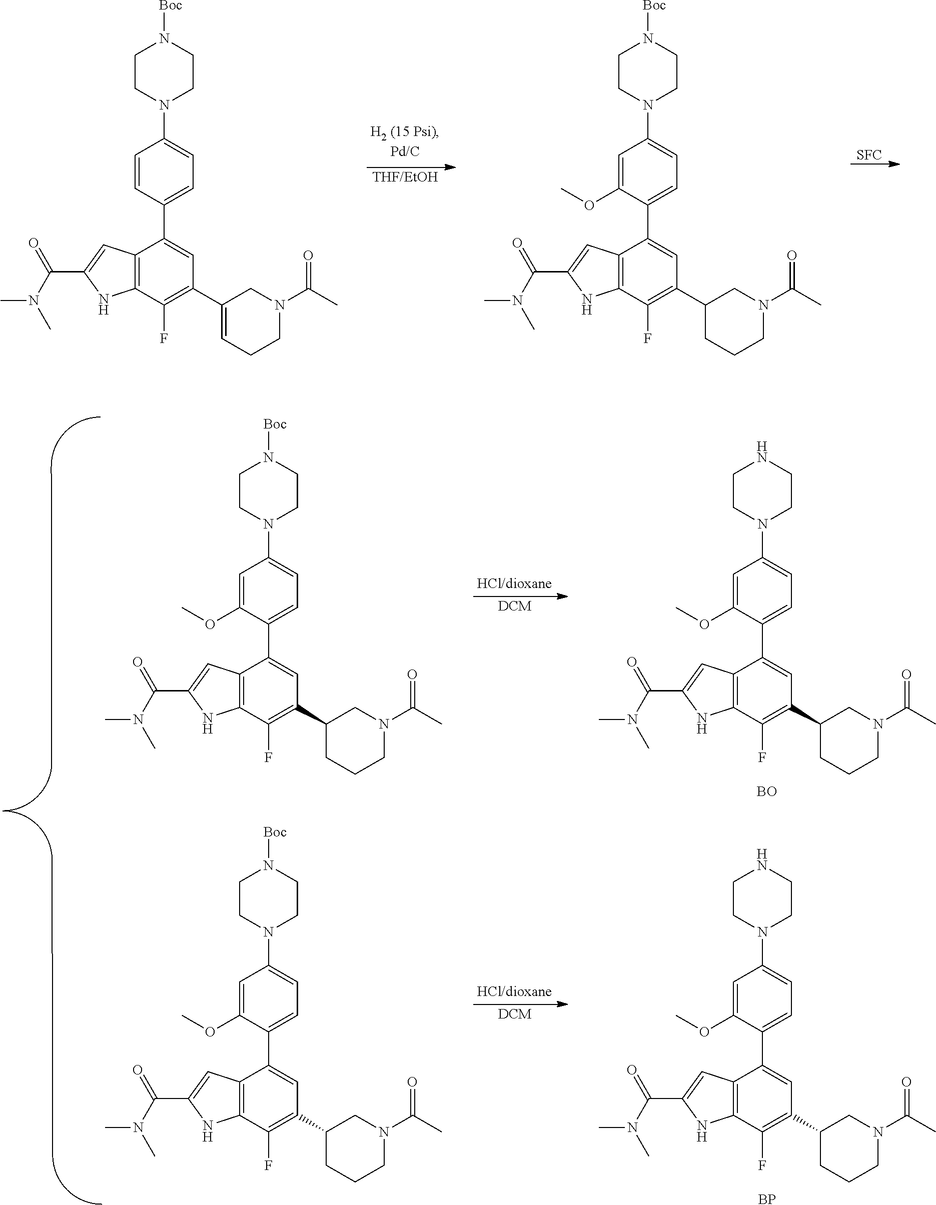

### Step 1—Tert-butyl 4-(4-(6-(1-acetylpiperidin-3-yl)-2-(dimethylcarbamoyl)-7-fluoro-1H-indol-4-yl)-3-methoxyphenyl)piperazine-1-carboxylate

To a solution of tert-butyl 4-(4-(6-(1-acetyl-1,2,5,6-tetrahydropyridin-3-yl)-2-(dimethylcarbamoyl)-7-fluoro-1H-indol-4-yl)-3-methoxyphenyl)piperazine-1-carboxylate (5.00 g, 8.07 mmol, synthesized via Steps 1-2 of Intermediate F) in CF₃CH₂OH (50.0 mL) was added PtO₂ (2.50 g, 11.01 mmol) under N₂. Then the mixture was stirred at 25° C. for 5 h under H₂(15 psi), On completion, the mixture was filtered and the filtrate was concentrated. The residue was purified by prep-HPLC (column: Phenomenex luna C18 250*50 mm*10 um; mobile phase: [water(FA)-ACN]; gradient:25%-55% B over 5 min) to give the title compound (6.11 g, 57% yield) as a yellow solid. LC-MS (ESI⁺) m/z 522.3 (M-100+H) ⁺.

### Step 2—Tert-butyl (S)-4-(4-(6-(1-acetylpiperidin-3-yl)-2-(dimethylcarbamoyl)-7-fluoro-1H-indol-4-yl)-3-methoxyphenyl)piperazine-1-carboxylate and tert-butyl (R)-4-(4-(6-(1-acetylpiperidin-3-yl)-2-(dimethylcarbamoyl)-7-fluoro-1H-indol-4-yl)-3-methoxyphenyl)piperazine-1-carboxylate

Tert-butyl 4-(4-(6-(1-acetylpiperidin-3-yl)-2-(dimethylcarbamoyl)-7-fluoro-1H-indol-4-yl)-3-methoxyphenyl)piperazine-1-carboxylate was purified by SFC (column: (s,s) WHELK-O1 (250 mm*50 mm, 10 um); mobile phase: [CO₂-ACN/EtOH(0.1% NH₃H₂O)]; B %: 65%, isocratic elution mode) and further SFC(column: REGIS(S,S)WHELK-O1(250 mm* 25 mm, 10 um); mobile phase: [CO₂-ACN/EtOH(0.1% NH₃H₂O)]; B %: 60%, isocratic elution mode) to give tert-butyl (S)-4-(4-(6-(1-acetylpiperidin-3-yl)-2-(dimethylcarbamoyl)-7-fluoro-1H-indol-4-yl)-3-methoxyphenyl)piperazine-1-carboxylate (2.52 g, 41% yield) as a white solid (LC-MS (ESI⁺) m/z 622.6 (M+H)⁺; ¹H NMR (400 MHz, DMSO-d₆) δ ppm 11.85 (s, 1H), 7.17 (d, J=8.4 Hz, 1H), 6.88 (dd, J=6.0, 10.4 Hz, 1H), 6.69 (d, J=2.0 Hz, 1H), 6.61 (d, J=7.6 Hz, 1H), 6.45 (d, J=2.8 Hz, 1H), 4.55-4.34 (m, 1H), 3.91-3.79 (m, 1H), 3.71 (s, 3H), 3.48 (d, J=4.8 Hz, 3H), 3.28-3.15 (m, 6H), 3.14-2.85 (m, 6H), 2.74-2.53 (m, 1H), 2.03 (d, J=9.6 Hz, 3H), 1.95-1.88 (m, 1H), 1.87-1.68 (m, 2H), 1.65-1.46 (m, 1H), 1.43 (s, 9H) and tert-butyl (R)-4-(4-(6-(1-acetylpiperidin-3-yl)-2-(dimethylcarbamoyl)-7-fluoro-1H-indol-4-yl)-3-methoxyphenyl)piperazine-1-carboxylate (2.06 g, 34% yield) as a white solid (LC-MS (ESI⁺) m/z 622.6 (M+H)⁺; ¹H NMR (400 MHz, DMSO-d₆) δ ppm 12.16-10.52 (m, 1H), 7.17 (d, J=8.4 Hz, 1H), 6.88 (dd, J=6.0, 10.4 Hz, 1H), 6.69 (d, J=2.0 Hz, 1H), 6.61 (d, J=8.4 Hz, 1H), 6.45 (d, J=2.8 Hz, 1H), 4.54-4.38 (m, 1H), 3.85 (d, J=13.6 Hz, 1H), 3.71 (s, 3H), 3.49 (d, J=4.8 Hz, 4H), 3.28-3.16 (m, 6H), 3.15-2.89 (m, 6H), 2.73-2.53 (m, 1H), 2.08-1.99 (m, 3H), 1.92 (s, 1H), 1.88-1.66 (m, 2H), 1.64-1.47 (m, 1H), 1.43 (s, 9H)). The absolute stereochemistry of the enantiomers was assigned arbitrarily.

### Step 3—(S)-6-(1-acetylpiperidin-3-yl)-7-fluoro-4-(2-methoxy-4-(piperazin-1-yl)phenyl)-N,N-dimethyl-1H-indole-2-carboxamide

A mixture of tert-butyl (S)-4-(4-(6-(1-acetylpiperidin-3-yl)-2-(dimethylcarbamoyl)-7-fluoro-1H -indol-4-yl)-3-methoxyphenyl)piperazine-1-carboxylate (1.36 g, 2.19 mmol) and HCl/dioxane (4.0 M, 14.0 mL) in DCM (14.0 mL) and EtOH (5.0 mL) was stirred at 20° C. for 1 hr. On completion, the mixture was concentrated to give the title compound (1.52 g, 99% yield, HCl) as a yellow solid. LC-MS (ESI⁺) m/z 522.5 (M+H)⁺.

### Step 4—(R)-6-(1-acetylpiperidin-3-yl)-7-fluoro-4-(2-methoxy-4-(piperazin-1-yl)phenyl)-N,N-dimethyl-1H-indole-2-carboxamide

A mixture of tert-butyl (R)-4-(4-(6-(1-acetylpiperidin-3-yl)-2-(dimethylcarbamoyl)-7-fluoro-1H-indol-4-yl)-3-methoxyphenyl)piperazine-1-carboxylate (620 mg, 997 μmol) and HCl/dioxane (4.0 M, 6.0 mL) in DCM (6.0 mL) and EtOH (3.0 mL) was stirred at 20° C. for 1 hr. On completion, the mixture was concentrated to give the title compound (780 mg, 98% yield, HCl) as a yellow solid. LC-MS (ESI⁺) m/z 522.4 (M+H)⁺.

### 3-(4-(4-(4-Chloro-3-methylphenyl)piperidin-1-yl)-3-methyl-1H-indazol-1-yl)piperidine-2,6-dione (Intermediate BQ)

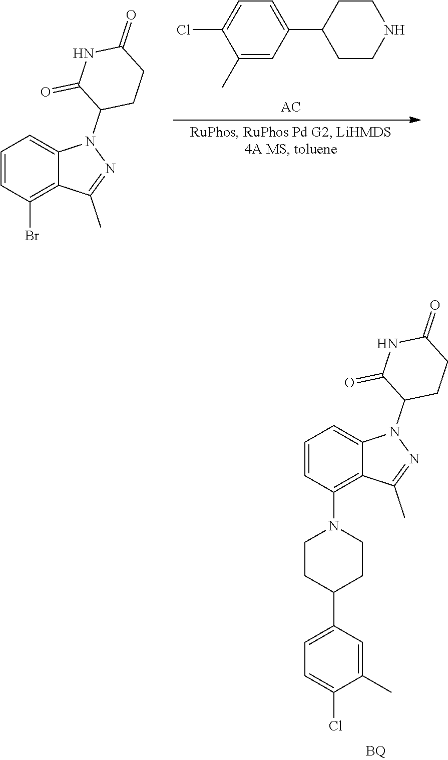 [SMILES: Cc1nn(C2CCC(=O)NC2=O)c2cccc(Br)c12 ; Cc1cc(C2CCNCC2)ccc1Cl ; Cc1cc(C2CCN(c3cccc4c3c(C)nn4C3CCC(=O)NC3=O)CC2)ccc1Cl]

To a solution of 3-(4-bromo-3-methyl-1H-indazol-1-yl)piperidine-2,6-dione (400 mg, 1.24 mmol, synthesized via Step 1 of Intermediate AB) in anhydrous toluene (8 mL) was added 4 Å molecular sieves (1.5 g), 4-(4-chloro-3-methylphenyl)piperidine (611 mg, 2.48 mmol, HCl, Intermediate AC), and LiHMDS (1 M, 7.45 mL) at 0° C. under nitrogen atmosphere. Then RuPhos (86.9 mg, 186 μmol) and [2-(2-aminophenyl)phenyl]-chloro-palladium dicyclohexyl-[2-(2,6-diisopropoxyphenyl)phenyl]phosphane (96.4 mg, 124 μmol) was added and the reaction was stirred at 100° C. for 1 hr under nitrogen atmosphere. On completion, the reaction mixture was quenched by HCOOH at 0° C. until the pH=6. Then the mixture was filtered and the filtrate was concentrated in vacuo to give the crude residue. The crude residue was purified by reverse phase HPLC (0.8 g/L ammonium bicarbonate) to give the title compound (120 mg, 21% yield) as a white solid. LC-MS (ESI⁺) m/z 451.2 (M+H)⁺.

### 3-((4-(4-(4-Chloro-3-methylphenyl)piperidin-1-yl)-5-fluoro-2-methoxyphenyl)amino)piperidine-2,6-dione (Intermediate BR)

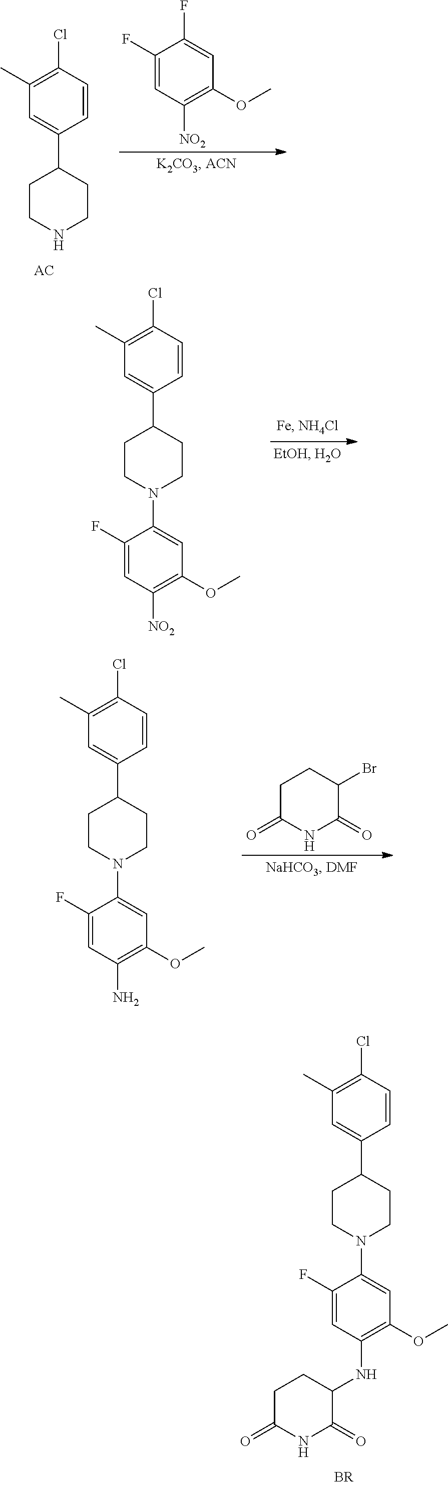

### Step 1—4-(4-chloro-3-methylphenyl)-1-(2-fluoro-5-methoxy-4-nitrophenyl)piperidine

To a solution of 1,2-difluoro-4-methoxy-5-nitrobenzene (460 mg, 2.44 mmol, CAS #66684-64-8) and 4-(4-chloro-3-methylphenyl)piperidine (600 mg, 2.44 mmol, HCl, Intermediate AC) in ACN (10 mL) was added K₂CO₃ (1.01 g, 7.31 mmol). The mixture was then stirred at 80° C. for 2 hrs. On completion, the reaction mixture was filtered and the filtrate was concentrated in vacuo to give the crude residue. The residue was purified by column chromatography (SiO₂, Petroleum ether/Ethyl acetate=1/0 to 1/1) to give the title compound (600 mg, 65% yield) as a yellow oil. LC-MS (ESI⁺) m/z 378.9 (M+H)⁺.

### Step 2—4-(4-(4-Chloro-3-methylphenyl)piperidin-1-yl)-5-fluoro-2-methoxyaniline

To a solution of 4-(4-chloro-3-methylphenyl)-1-(2-fluoro-5-methoxy-4-nitrophenyl)piperidine (600 mg, 1.58 mmol) in EtOH (3 mL) and H₂O (3 mL) was added Fe (619 mg, 11.0 mmol) and NH₄Cl (593 mg, 11.0 mmol). The mixture was then stirred at 50° C. for 2 hrs. On completion, the reaction mixture was filtered and the filtrate was concentrated in vacuo to give the title compound (345 mg) as a yellow solid. LC-MS (ESI⁺) m/z 349.1 (M+H)⁺.

### Step 3—3-((4-(4-(4-Chloro-3-methylphenyl)piperidin-1-yl)-5-fluoro-2-methoxyphenyl)amino)piperidine-2,6-dione

To a solution of 4-[4-(4-chloro-3-methyl-phenyl)-1-piperidyl]-5-fluoro-2-methoxy-aniline (345 mg, 989 μmol) and 3-bromopiperidine-2,6-dione (398 mg, 2.08 mmol, CAS #62595-74-8) in DMF (6 mL) was added NaHCO₃ (249 mg, 2.97 mmol). The mixture was then stirred at 80° C. for 6 hrs. On completion, the reaction mixture was diluted with H₂O (60 mL) and extracted with EtOAc (50 mL×2). The combined organic layers were washed with brine (100 mL), dried over anhydrous sodium sulfate, filtered and concentrated under reduced pressure to give a residue. The residue was purified by column chromatography (SiO₂, Petroleum ether/Ethyl acetate=1/0 to 0/1) to give the title compound (310 mg, 68% yield) as a yellow solid. LC-MS (ESI⁺) m/z 459.1 (M+H)⁺.

### 3-(4-(4-(4-chloro-3-methoxyphenyl)piperidin-1-yl)-3-methyl-1H-indazol-1-yl)piperidine-2,6-dione (Intermediate BS)

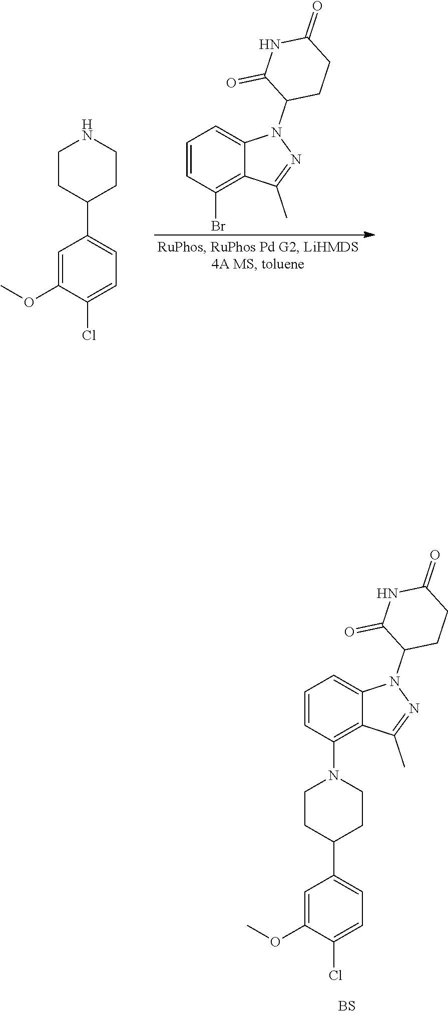

A mixture of 3-(4-bromo-3-methyl-1H-indazol-1-yl)piperidine-2,6-dione (614 mg, 1.91 mmol, synthesized via Step 1 of Intermediate AB), 4-(4-chloro-3-methoxyphenyl)piperidine (500 mg, 1.91 mmol, HCl, CAS #2168546-59-4) and 4A molecular sieves (2 g) in toluene (11 mL) was added LiHMDS (1 M, 11.4 mL) at 0° C. Then RuPhos (133 mg, 286 μmol) and [2-(2-aminophenyl)phenyl]-chloro-palladium; dicyclohexyl-[2-(2,6-diisopropoxyphenyl)phenyl]phosphane (148 mg, 190 μmol) was added and the reaction was stirred at 100° C. for 1 hr under nitrogen atmosphere. On completion, the reaction mixture was quenched with HCOOH at 0° C. until pH=6. The mixture was filtered and the filtrate was concentrated in vacuo to give the crude residue. The crude residue was purified by reverse phase HPLC (0.8 g/L ammonium bicarbonate) to give the title compound (300 mg, 33% yield) as a white solid. LC-MS (ESI⁺) m/z 467.0 (M+H)⁺.

### 3-[4-[4-(4-Chlorophenyl)-1-piperidyl]-3-fluoro-phenyl]piperidine-2,6-dione (Intermediate BT)

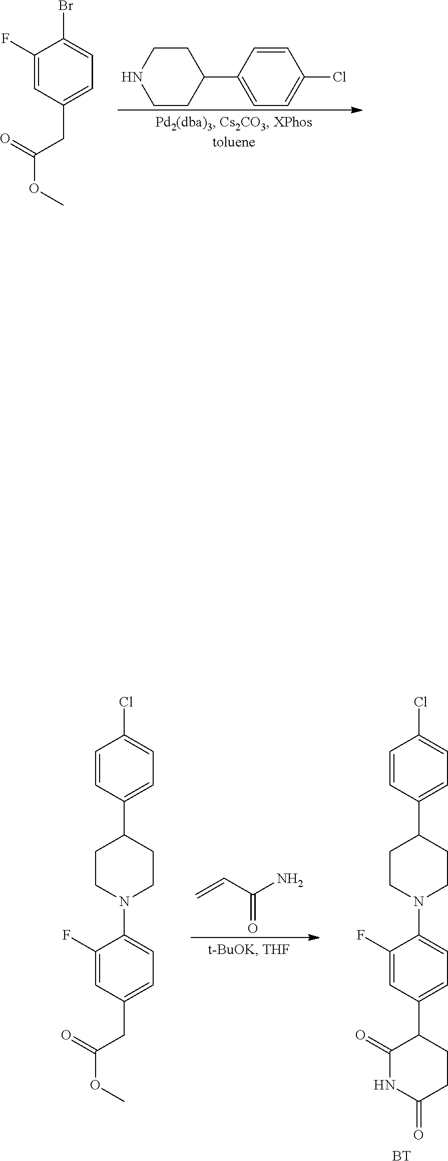

### Step 1—Methyl 2-[4-[4-(4-chlorophenyl)-1-piperidyl]-3-fluoro-phenyl]acetate

A mixture of methyl 2-(4-bromo-3-fluoro-phenyl)acetate (500 mg, 2.02 mmol, CAS #942282-41-9), 4-(4-chlorophenyl)piperidine (396 mg, 2.02 mmol, CAS #26905-02-2), Pd₂(dba)₃ (92 mg, 101 μmol), Cs₂CO₃ (1.98 g, 6.07 mmol) and XPhos (115 mg, 242 mol) in toluene (6.0 mL) was degassed and purged with N₂ three times. Then the mixture was stirred at 100° C. for 2 h under N₂ atmosphere. On completion, the mixture was concentrated under reduced pressure using a rotary evaporator to give the title compound (750 mg, 92% yield) as a white solid. LC-MS (ESI⁺) m/z 362.1 (M+H)⁺.

### Step 2—3-[4-[4-(4-Chlorophenyl)-1-piperidyl]-3-fluoro-phenyl]piperidine-2,6-dione

A mixture of methyl 2-[4-[4-(4-chlorophenyl)-1-piperidyl]-3-fluoro-phenyl]acetate (380 mg, 1.05 mmol) and prop-2-enamide (74.0 mg, 1.05 mmol, 72 μL) in THF (5.0 mL) was degassed and purged with N₂ three times. Then t-BuOK (1 M, 1.26 mL) was added at 0° C. and the mixture was stirred at 50° C. for 0.5 h under N₂ atmosphere. On completion, the mixture was diluted with NH₄Cl (15 mL) and extracted with ethyl acetate (15 mL×2), dried over sodium sulfate, filtered and concentrated under reduced pressure. The crude product was purified by prep-HPLC (column: Waters Xbridge BEH C18 250*50 mm*10 um; mobile phase: [water (NH₄HCO₃)-ACN]; gradient:55%-85% B over 10 min) to give the title compound (160 mg, 34% yield) as a white solid. LC-MS (ESI⁺) m/z 401.2 (M+H)⁺.

### 4-(4-Chloro-2-fluoro-5-methoxyphenyl)piperidine (Intermediate BU)

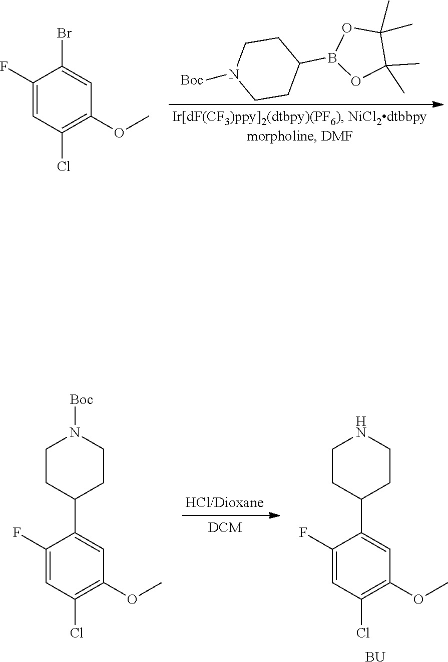

### Step 1—Tert-butyl 4-(4-chloro-2-fluoro-5-methoxyphenyl)piperidine-1-carboxylate

To a solution of 1-bromo-4-chloro-2-fluoro-5-methoxybenzene (7 g, 29.2 mmol, CAS #146447-18-9) in DMF (292 mL) was added Ir[dF(CF₃)ppy]₂(dtbpy)(PF₆) (327 mg, 292 mol), NiCl₂·dtbbpy (581 mg, 1.46 mmol), morpholine (3.82 g, 43.8 mmol, 3.86 mL) and tert-butyl 4-(4,4,5,5-tetramethyl-1,3,2-dioxaborolan-2-yl)piperidine-1-carboxylate (18.1 g, 58.4 mmol, CAS #1048970-17-7). The reaction solution was pumped through a reactor at a flow rate of 0.3 mL/min, irradiating with a 450 nm LED lamp, with cooling fan to keep the reaction temperature at 25° C. for 14 h. On completion, the mixture was diluted with H₂O (300 mL) and extracted with EA (500 mL×3). The combined organic layers were washed with aqueous NaCl (1000 mL×3), dried over Na₂SO₄, filtered and concentrated under reduced pressure to give a residue. The residue was purified by reverse phase HPLC (0.1% FA condition) to give the title compound (3 g, 16% yield, FA) as a black solid. LC-MS (ESI⁺) m/z 288.1 (M-55)⁺.

### Step 2—4-(4-Chloro-2-fluoro-5-methoxyphenyl)piperidine

To a solution of tert-butyl 4-(4-chloro-2-fluoro-5-methoxyphenyl)piperidine-1-carboxylate (1 g, 2.91 mmol) in DCM (10 mL) was added HCl/dioxane (4 M, 4 mL). The mixture was then stirred at 25° C. for 2 hrs. On completion, the mixture was concentrated under reduced pressure. The residue was purified by reverse phase HPLC (0.8 g/L ammonium bicarbonate) to give the title compound (600 mg, 85% yield) as a black solid. LC-MS (ESI⁻) m/z 244.1 (M+H)⁺.

### 3-(4-(4-(4-Chloro-2-fluoro-5-methoxyphenyl)piperidin-1-yl)-3-methyl-2-oxo-2,3-dihydro-1H-benzo[d]imidazol-1-yl)piperidine-2,6-dione (Intermediate BV)

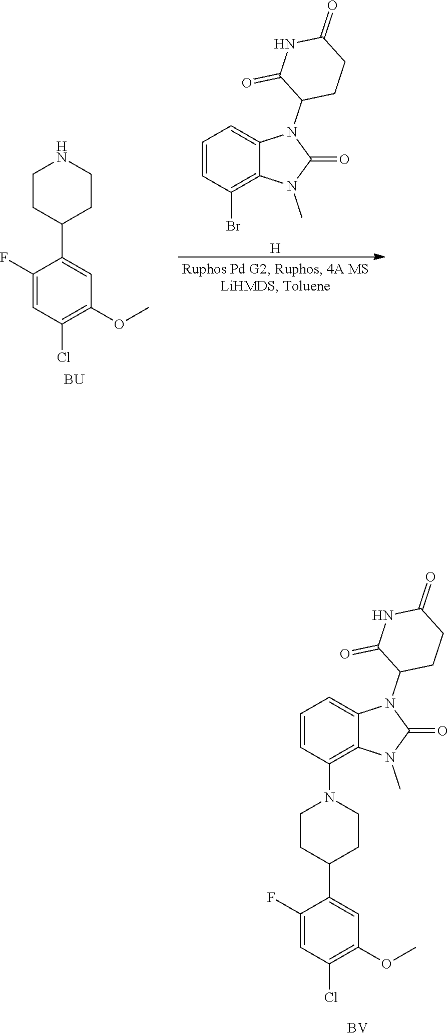

A mixture of 4-(4-chloro-2-fluoro-5-methoxyphenyl)piperidine (480 mg, 1.97 mmol, Intermediate BU), 3-(4-bromo-3-methyl-2-oxo-2,3-dihydro-1H-benzo[d]imidazol-1-yl)piperidine-2,6-dione (555 mg, 1.64 mmol, Intermediate H) in toluene (5 mL) at 0° C. was added LiHMDS (1 M, 9.85 mL), RuPhos (114 mg, 246 mol), [2-(2-aminophenyl)phenyl]-chloro-palladium dicyclohexyl-[2-(2,6-diisopropoxyphenyl)phenyl]phosphane (127 mg, 164 μmol), and 4 Å molecular sieves (100 mg) was degassed and purged with N₂ three times. Then the mixture was stirred at 100° C. for 2 hrs under N₂ atmosphere. On completion, the mixture was concentrated under reduced pressure, purified by reverse phase HPLC (0.8 g/L ammonium bicarbonate) to give the title compound (150 mg, 17% yield) as a white solid. LC-MS (ESI⁺) m/z 500.9 (M+H)⁺.

### 4-(4-Chloro-2-fluoro-5-methylphenyl)piperidine (Intermediate BW)

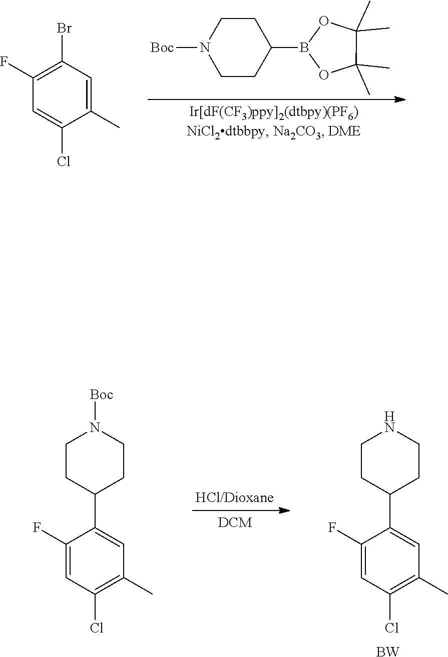

### Step 1—Tert-butyl 4-(4-chloro-2-fluoro-5-methylphenyl)piperidine-1-carboxylate

To a solution of 1-bromo-4-chloro-2-fluoro-5-methyl-benzene (5 g, 22.3 mmol, CAS #200190-87-0), tert-butyl 4-(4,4,5,5-tetramethyl-1,3,2-dioxaborolan-2-yl)piperidine-1-carboxylate (10.4 g, 33.5 mmol, CAS #1048970-17-7) in DME (50 mL) was added bis[3,5-difluoro-2-[5-(trifluoromethyl)-2-pyridyl]phenyl]iridium(1+) 4-tert-butyl-2-(4-tert-butyl-2-pyridyl)pyridine hexafluorophosphate (0.25 g, 0.22 mmol), Na₂CO₃ (12.7 g, 44.7 mmol), and 4-tert-butyl-2-(4-tert-butyl-2-pyridyl)pyridine dichloronickel (132 mg, 0.335 mmol). The reaction solution was pumped through a reactor at a flow rate of 0.3 mL/min, irradiating with a 450 nm LED lamp, with cooling fan to keep the reaction temperature at 25° C. for 14 h. On completion, the reaction mixture was diluted with EA (150 mL) and washed with water (3×50 mL). The combined organic layers were washed with brine (50 mL) and dried over anhydrous sodium sulfate, filtered and the filtrate was concentrated in vacuo to give the crude residue. The crude residue was purified by reverse phase HPLC (0.1% FA condition) to give the title compound to give the title compound (2.1 g, 29% yield) as a white solid. LC-MS (ESI⁺) m/z 272.1 (M-55)⁺.

### Step 2—4-(4-Chloro-2-fluoro-5-methylphenyl)piperidine

To a solution of tert-butyl 4-(4-chloro-2-fluoro-5-methyl-phenyl)piperidine-1-carboxylate (1 g, 3 mmol) in DCM (10 mL) was added HCl/dioxane (4 M, 3 mL). The mixture was then stirred at 25° C. for 1 hr. On completion, the reaction mixture was concentrated under reduced pressure to remove DCM and HCl/dioxane to give the title compound (900 mg) as a white solid. LC-MS (ESI⁺) m/z 228.0 (M+H)⁺.

### 3-(4-(4-(4-Chloro-2-fluoro-5-methylphenyl)piperidin-1-yl)-3-methyl-2-oxo-2,3-dihydro-1H-benzo[d]imidazol-1-yl)piperidine-2,6-dione (Intermediate BX)

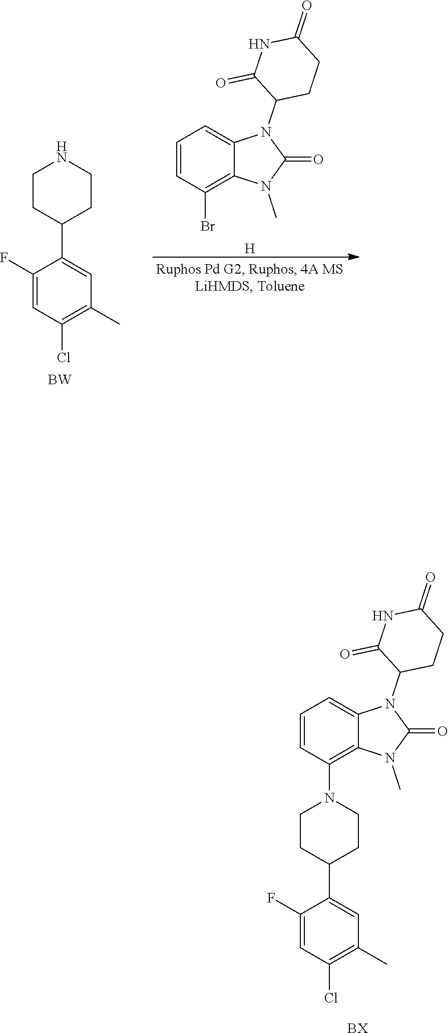

A mixture of 4-(4-chloro-2-fluoro-5-methyl-phenyl)piperidine (703 mg, 2.6 mmol, HCl, Intermediate BW), 3-(4-bromo-3-methyl-2-oxo-benzimidazol-1-yl)piperidine-2,6-dione (750 mg, 2.22 mmol, Intermediate H), RuPhos (104 mg, 222 μmol), 4A molecular sieves (800 mg), [2-(2-aminophenyl)phenyl]-chloro-palladium dicyclohexyl-[2-(2,6-diisopropoxyphenyl)phenyl]phosphane (172 mg, 222 μmol) and LiHMDS (1 M, 13.3 mL) in toluene (7 mL) was degassed and purged with N₂ three times. Then the mixture was stirred at 25° C. for 1 hr under N₂ atmosphere. On completion, the reaction mixture was quenched by addition of NH₄Cl solution (10 mL) at 0° C., and then diluted with water (20 mL) and extracted with ethyl acetate (20 mL×2). The combined organic layers were washed with brine (10 mL×2), dried over Na₂SO₄, filtered and concentrated under reduced pressure to give a residue. The residue was purified by prep-HPLC (0.10% FA) to give the title compound (314 mg, 29% yield) as a pink solid. LC-MS (ESI⁺) m/z 485.1 (M+H).

### 6-(1-Acetyl-1,2,5,6-tetrahydropyridin-3-yl)-7-fluoro-N,N-dimethyl-4-(4,4,5,5-tetramethyl-1,3,2-dioxaborolan-2-yl)benzo[b]thiophene-2-carboxamide (Intermediate BY)

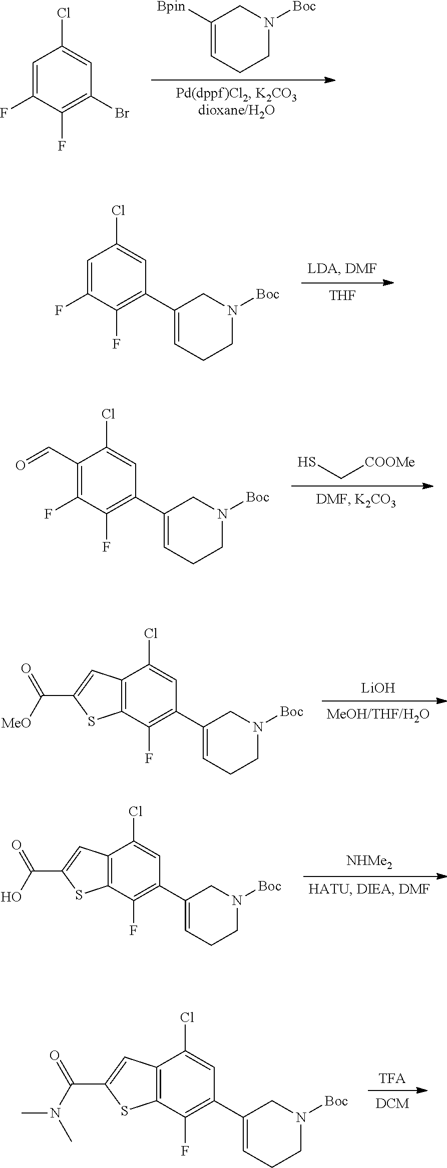

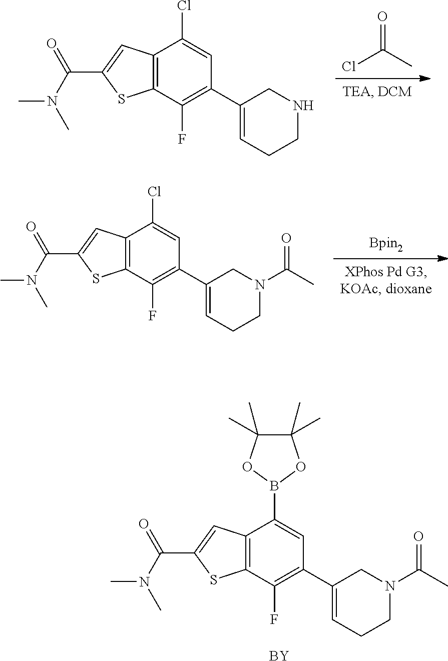

### Step 1—Tert-butyl 5-(5-chloro-2,3-difluorophenyl)-3,6-dihydropyridine-1(2H)-carboxylate

A mixture of 1-bromo-5-chloro-2,3-difluoro-benzene (5 g, 22.0 mmol, CAS #1160573-26-1), tert-butyl 5-(4,4,5,5-tetramethyl-1,3,2-dioxaborolan-2-yl)-3,6-dihydro-2H-pyridine-1-carboxylate (7.48 g, 24.2 mmol CAS #885693-20-9). Pd(dppf)Cl₂·CH₂Cl₂ (1.80 g, 2.20 mmol), and K₂CO₃ (9.12 g, 65.9 mmol) in dioxane (40 mL) and H₂O (10 mL) was degassed and purged with N₂ three times. Then the mixture was stirred at 80° C. for 2 hrs under N₂ atmosphere. On completion, the reaction mixture was diluted with water (10 mL) and extracted with dichloromethane (2×20 mL). The combined organic layers were washed with brine (20 mL) and dried over anhydrous sodium sulfate, filtered and the filtrate was concentrated in vacuo to give the crude residue. The residue was purified by column chromatography (SiO₂, Petroleum ether:Ethyl acetate=10/1) to give the title compound (7 g, 96% yield) as a yellow solid. LC-MS (ESI⁺) m/z 274.0 (M-55)⁻.

### Step 2—Tert-butyl 5-(5-chloro-2,3-difluoro-4-formylphenyl)-3,6-dihydropyridine-1(2H)-carboxylate

A mixture of tert-butyl 5-(5-chloro-2,3-difluoro-phenyl)-3,6-dihydro-2H-pyridine-1-carboxylate (6.00 g, 18.2 mmol) in dry THF (50 mL) was degassed and purged with N₂ three times, then LDA (2 M, 10.9 mL) was added at −78° C., then the mixture was stirred at −78° C. for 2 hrs under N₂ atmosphere. Next. DMF (2.66 g, 36.4 mmol) was added and the mixture was stirred at −78° C. for 2 hrs under N₂ atmosphere. On completion, the reaction mixture was quenched with sat. NH₄Cl (10 mL) and extracted with EtOAc (3×50 mL). The combined organic layers were washed with brine (50 mL) and dried over anhydrous sodium sulfate, filtered and the filtrate was concentrated in vacuo to give the residue. The residue was purified by column chromatography (SiO₂, Petroleum ether:Ethyl acetate=10/1) to give the title compound (7 g, 96% yield) as a yellow solid. LC-MS (ESI⁺) m/z 302.0 (M-55)⁺.

### Step 3—Tert-butyl 5-(4-chloro-7-fluoro-2-(methoxycarbonyl)benzo[b]thiophen-6-yl)-3,6-dihydropyridine-1(4)-carboxylate

To a solution of tert-butyl 5-(5-chloro-2,3-difluoro-4-formyl-phenyl)-3,6-dihydro-2H-pyridine-1-carboxylate (4.5 g, 13 mmol) in DMF (80 mL) was added methyl 2-sulfanylacetate (1.60 g, 15.1 mmol, CAS #2365-48-2). Then K₂CO₃ (3.48 g, 25.2 mmol) was added and the mixture was stirred at 20° C. for 4 hrs. On completion, water (160 mL) was added and the mixture was filtered to give the filter cake as the title compound (6 g) as a yellow solid. LC-MS (ESI⁺) m/z 369.8 (M-55)⁺.

### Step 4—6-(1-(Tert-butoxycarbonyl)-1,2,5,6-tetrahydropyridin-3-yl)-4-chloro-7-fluorobenzo[b]thiophene-2-carboxylic acid

To a solution of tert-butyl 5-(4-chloro-7-fluoro-2-methoxycarbonyl-benzothiophen-6-yl)-3,6-dihydro-2H-pyridine-1-carboxylate (5.50 g, 12.9 mmol) in THF (8 mL). MeOH (2 mL) and H₂O (2 mL) was added LiOH·H₂O (1.63 g, 38.7 mmol). Then the mixture was stirred at 40° C. for 2 hrs. On completion, the reaction mixture was added 6M HCl to adjust the pH to 3-4, and then extracted with DCM (10 mL×3). The combined organic layers were washed with brine (10 mL), dried over Na₂SO₄, filtered and concentrated under reduced pressure to give the title compound (5.5 g) as a yellow solid. LC-MS (ESI⁺) m/z 355.8 (M-55)⁻.

### Step 5—Tert-butyl 5-(4-chloro-2-(dimethylcarbamoyl)-7-fluorobenzo[b]thiophen-6-yl)-3,6-dihydropyridine-1(2H)-carboxylate

To a solution of 6-(1-tert-butoxycarbonyl-3,6-dihydro-2H-pyridin-5-yl)-4-chloro-7-fluoro-benzothiophene-2-carboxylic acid (5.00 g, 12.1 mmol) in DMF (30 mL) was added DIEA (7.84 g, 60.7 mmol) and HATU (5.08 g, 13.4 mmol). Then N-methylmethanamine (2.97 g, 36.4 mmol, HCl) was added and the mixture was stirred at 25° C. for 1 hr. On completion, the reaction mixture was quenched by addition of H₂O (10 mL), and then extracted with EtOAc (30 mL×3). The combined organic layers were washed with brine (30 mL×3), dried over Na₂SO₄, filtered and concentrated under reduced pressure to give the title compound (5.5 g) as a yellow solid. LC-MS (ESI⁺) m/z 439.0 (M+H)⁺.

### Step 6—4-Chloro-7-fluoro-N,N-dimethyl-6-(1,2,5,6-tetrahydropyridin-3-yl)benzo[b]thiophene-2-carboxamide

To a solution of tert-butyl 5-[4-chloro-2-(dimethylcarbamoyl)-7-fluoro-benzothiophen-6-yl]-3,6-dihydro-2H-pyridine-1-carboxylate (5.50 g, 12.5 mmol) in DCM (40 mL) was added TFA (15.4 g, 135 mmol). The mixture was then stirred at 25° C. for 1 hr. On completion, the reaction mixture was concentrated under N₂ to remove solvent. The reaction mixture was quenched by addition of sat. NaHCO₃ to adjust the pH to 7-8, and then extracted with DCM (20 mL×3). The combined organic layers were washed with brine (20 mL), dried over Na₂SO₄, filtered and concentrated under reduced pressure to give the title compound (5.0 g) as a yellow solid. LC-MS (ESI⁺) m/z 339.0 (M+H).

### Step 7—6-(1-Acetyl-1,2,5,6-tetrahydropyridin-3-yl)-4-chloro-7-fluoro-N,N-dimethylbenzo[b]thiophene-2-carboxamide

To a solution of 4-chloro-7-fluoro-N,N-dimethyl-6-(1,2,3,6-tetrahydropyridin-5-yl)benzothiophene-2-carboxamide (3.80 g, 11.2 mmol) in DCM (35 mL) was added TEA (4.54 g, 44.9 mmol). Then acetyl chloride (2.64 g, 33.7 mmol) was added and the mixture was stirred at 0-25° C. for 1 hrs. On completion, the reaction mixture was quenched by addition of H₂O (5 mL), and then extracted with DCM (20 mL×3). The combined organic layers were washed with brine (20 mL), dried over Na₂SO₄, filtered and concentrated under reduced pressure to give a residue. The residue was purified by silica gel column chromatography (Petroleum ether/Ethyl acetate=3/1 to DCM/Ethyl acetate=3/1) to give the title compound (2.3 g, 52% yield) as a yellow solid. LC-MS (ESI⁺) m/z 380.9 (M+H)⁺.

### Step 8—6-(1-Acetyl-1,2,5,6-tetrahydropyridin-3-yl)-7-fluoro-N,N-dimethyl-4-(4,4,5,5-tetramethyl-1,3,2-dioxaborolan-2-yl)benzo[b]thiophene-2-carboxamide

A mixture of 6-(1-acetyl-3,6-dihydro-2H-pyridin-5-yl)-4-chloro-7-fluoro-N,N-dimethyl-benzothiophene-2-carboxamide (2.20 g, 5.78 mmol), 4,4,5,5-tetramethyl-2-(4,4,5,5-tetramethyl-1,3,2-dioxaborolan-2-yl)-1,3,2-dioxaborolane (2.93 g, 11.6 mmol), KOAc (1.70 g, 17.3 mmol), and XPhos Pd G3 (978 mg, 1.16 mmol) in dioxane (15 mL) was degassed and purged with N₂ three times. Then the mixture was stirred at 80° C. for 2 hrs under N₂ atmosphere. On completion, the reaction mixture was filtered and concentrated under reduced pressure to give a residue. The residue was purified by silica gel column chromatography (Petroleum ether/Ethyl acetate=10:1 to DCM/Ethyl acetate=5/1) to give the title compound (1.70 g, 54% yield) as a yellow solid. LC-MS (ESI⁺) m/z 473.3 (M+H)⁺.

### (R)-6-(1-(3-(1H-pyrazol-1-yl)propanoyl)piperidin-3-yl)-N-cyclopropyl-7-fluoro-N-methyl-4-(4-(piperazin-1-yl)phenyl)-1H-indole-2-carboxamide (Intermediate BZ)

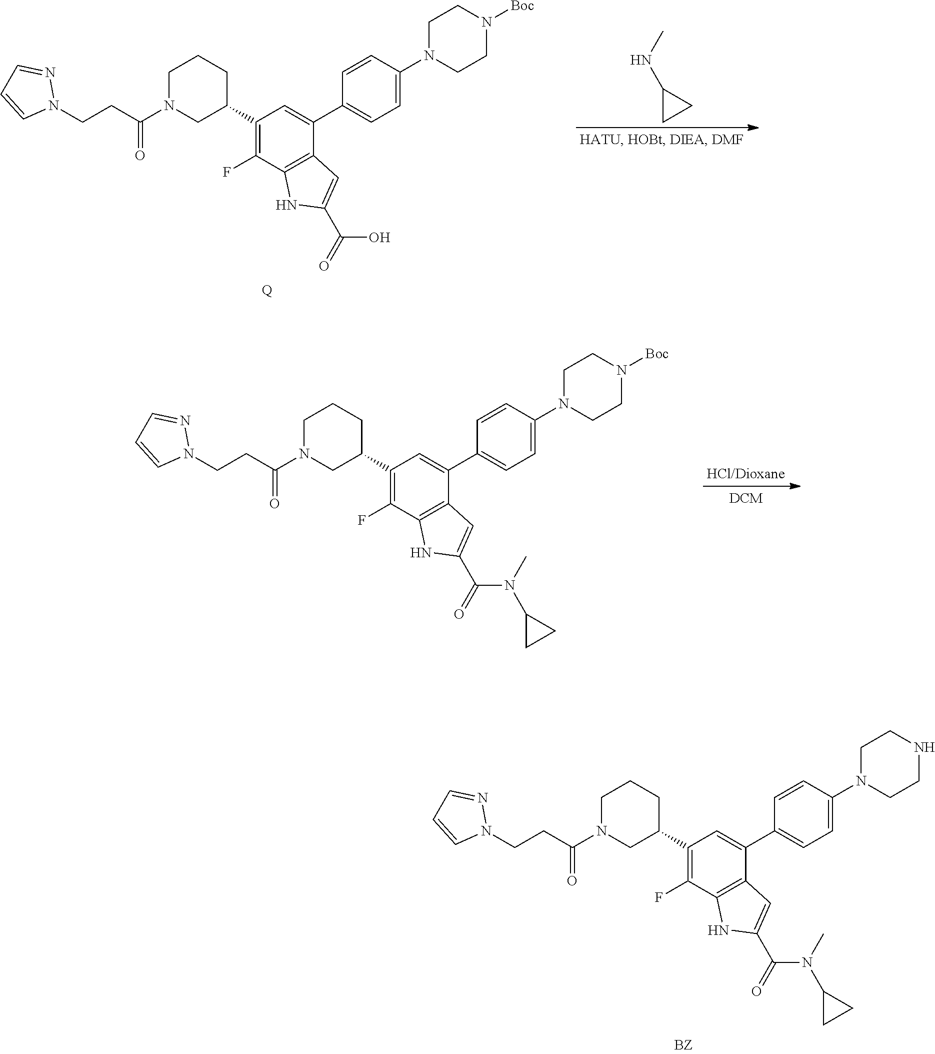

### Step 1—Tert-butyl (R)-4-(4-(6-(1-(3-(1H-pyrazol-1-yl)propanoyl)piperidin-3-yl)-2-(cyclopropyl(methyl)carbamoyl)-7-fluoro-1H-indol-4-yl)phenyl)piperazine-1-carboxylate

To a solution of (R)-6-(1-(3-(1H-pyrazol-1-yl)propanoyl)piperidin-3-yl)-4-(4-(4-(tert-butoxycarbonyl)piperazin-1-yl)phenyl)-7-fluoro-1H-indole-2-carboxylic acid (400 mg, 620 μmol, Intermediate Q) in DMF (4 mL) was added HATU (307 mg, 807 μmol), HOBt (168 mg, 1.24 mmol), DIEA (401 mg, 3.10 mmol, 540 μL) and N-methylcyclopropanamine (200 mg, 1.86 mmol, CAS #5163-20-2). The mixture was then stirred at 25° C. for 10 min. On completion, the crude residue was purified by reverse phase HPLC (0.1% FA condition) to give the title compound (350 g, 72% yield, FA) as a yellow solid. LC-MS (ESI⁺) m/z 698.3 (M+H)⁺.

### Step 2—(R)-6-(1-(3-(1H-pyrazol-1-yl)propanoyl)piperidin-3-yl)-N-cyclopropyl-7-fluoro-N-methyl-4-(4-(piperazin-1-yl)phenyl)-1H-indole-2-carboxamide

To a solution of tert-butyl (R)-4-(4-(6-(1-(3-(1H-pyrazol-1-yl)propanoyl)piperidin-3-yl)-2-(cyclopropyl(methyl)carbamoyl)-7-fluoro-1H-indol-4-yl)phenyl)piperazine-1-carboxylate (350 mg, 502 mol) in DCM (4 mL) was added HCl/Dioxane (4 M, 125 μL). The mixture was then stirred at 25° C. for 0.5 hrs. On completion, the reaction mixture was concentrated in vacuo to give the title compound (500 mg, HCl salt) as a yellow solid. LC-MS (ESI⁺) m/z 598.2 (M+H)⁺.

### 3-(1-(2,6-Dioxopiperidin-3-yl)-3-methyl-2-oxo-2,3-dihydro-1H-benzo[d]imidazol-4-yl)propanal (Intermediate CA)

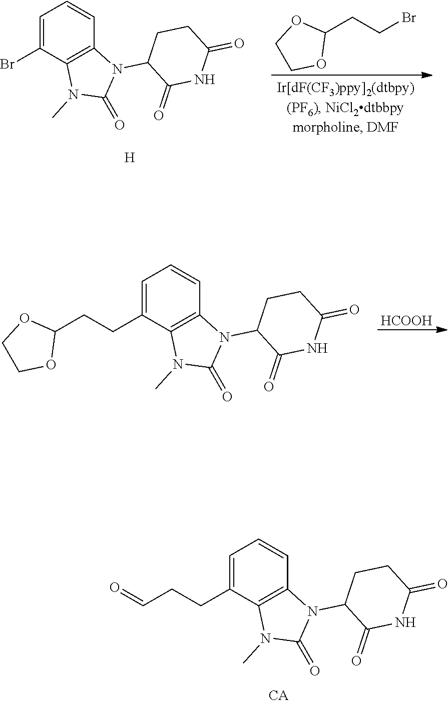

### Step 1—3-(4-(2-(1,3-Dioxolan-2-yl)ethyl)-3-methyl-2-oxo-2,3-dihydro-1H-benzo[d]imidazol-1-yl)piperidine-2,6-dione

To a solution of 3-(4-bromo-3-methyl-2-oxo-2,3-dihydro-1H-benzo[d]imidazol-1-yl)piperidine -2,6-dione (10 g, 29.5 mmol, Intermediate H), 2-(2-bromoethyl)-1,3-dioxolane (6.96 g, 38.4 mmol, 4.61 mL, CAS #18742-02-4), Ir[dF(CF₃)ppy]₂(dtbpy)(PF₆) (663 mg, 591 μmol), NiCl₂·dtbbpy (353 mg, 887 mol), TTMSS (8.82 g, 35.4 mmol, 10.9 mL), and 2,6-dimethylpyridine (28.5 g, 266 mmol, 31.0 mL) in DME (500 mL). The reaction was then stirred and irradiated with a 4×50 W [455 nm] blue LED lamp (3 cm away), with cooling water to keep the reaction temperature at 25° C. for 14 hrs. On completion, the reaction mixture was concentrated in vacuo to give the crude residue. The residue was purified by column chromatography (SiO₂, Petroleum ether:Ethyl acetate=1/0 to 0/1) to give the title compound (7 g, 62% yield) as a white solid. LC-MS (ESI⁺) m/z 360.2 (M+H)⁺.

### Step 2—3-(1-(2,6-Dioxopiperidin-3-yl)-3-methyl-2-oxo-2,3-dihydro-1H-benzo[d]imidazol-4-yl)propanal

To a solution of 3-(4-(2-(1,3-dioxolan-2-yl)ethyl)-3-methyl-2-oxo-2,3-dihydro-1H-benzo[d]imidazol-1-yl)piperidine-2,6-dione (300 mg, 834 μmol) in HCOOH (2 mL) was stirred at 40° C. for 1 hr. On completion, the reaction mixture was concentrated under reduced pressure to give the title compound (330 mg, FA) as a white solid. LC-MS (ESI⁺) m/z 316.0 (M+H)⁺.

### Methyl 2-bromo-4-chlorobenzo[d]thiazole-6-carboxylate (Intermediate CB)

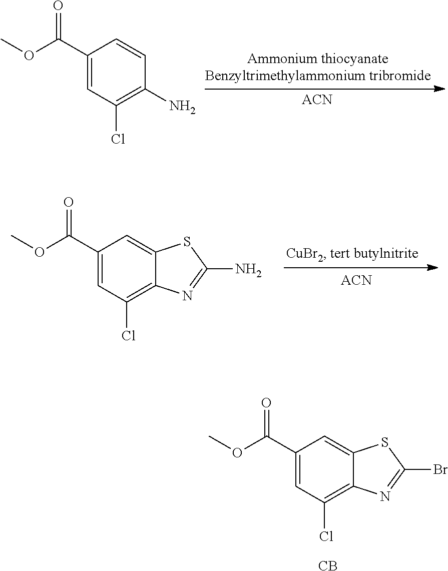

### Step 1—Methyl 2-amino-4-chlorobenzo[d]thiazole-6-carboxylate

To a solution of methyl 4-amino-3-chlorobenzoate (25 g, 134 mmol. CAS #84228-44-4) in ACN (360 mL) was added ammonia thiocyanic acid (15 g, 202 mmol. CAS #1762-95-4). After 10 min, Benzyltrimethylammonium tribromide (52 g, 134 mmol, CAS #111865-47-5) was added and the mixture was stirred at 25° C. for 12 hrs. On completion, the mixture was concentrated under reduced pressure. The residue was triturated with EA/PE=3/1 at 0° C. for 30 min, then filtered to give the title compound (20 g, 43% yield) as a yellow solid. LC-MS (ESI⁺) m/z 243.0 (M+H)⁺.

### Step 2—Methyl 2-bromo-4-chlorobenzo[d]thiazole-6-carboxylate

To a solution of methyl 2-amino-4-chlorobenzo[d]thiazole-6-carboxylate (1 g, 4 mmol) in ACN (8 mL) was added tert butylnitrite (637 mg, 6.18 mmol, 735 μL) and CuBr₂ (1.10 g, 4.94 mmol). The mixture was then stirred at 20° C. for 1 hr. On completion, the mixture was concentrated under reduced pressure. The residue was purified by reverse phase HPLC (0.1% FA condition) to give the title compound (0.6 g, 39% yield, FA) as a black solid. LC-MS (ESI⁺) m/z 307.7 (M+H)⁺.

### 1-(5-(4,4,5,5-Tetramethyl-1,3,2-dioxaborolan-2-yl)-3,6-dihydropyridin-1(2H)-yl)-3-(thiazol-2-yl)propan-1-one (Intermediate CC)

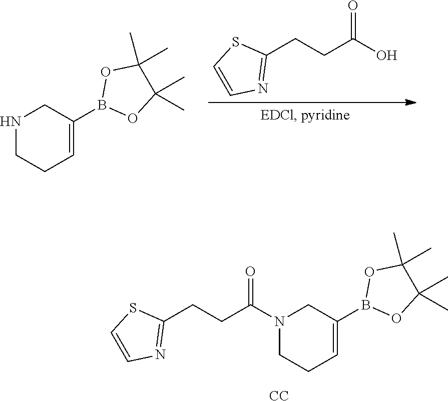

To a solution of 3-thiazol-2-ylpropanoic acid (3 g, 19.1 mmol, CAS #144163-65-5) and 5-(4,4,5,5-tetramethyl-1,3,2-dioxaborolan-2-yl)-1,2,3,6-tetrahydropyridine (5.16 g, 21.0 mmol, HCl, synthesized via Step 1 of Intermediate E) in pyridine (50 mL) was added EDCI (5.49 g, 28.6 mmol). The mixture was then stirred at 20° C. for 2 h. On completion, the reaction mixture was diluted with H₂O (50 ml) and extracted with EA (60 mL×3). The combined organic layers were dried over Na₂SO₄, filtered and concentrated under reduced pressure to give a residue. The residue was purified by column chromatography (SiO₂, Petroleum ether/Ethyl acetate=1/0 to 0/1) to give the title compound (3.9 g, 56% yield) as a yellow solid. ¹H NMR (400 MHz, DMSO-d₆) δ=7.66 (d, J=3.2 Hz, 1H), 7.54 (d, J=3.2 Hz, 1H), 6.61-6.47 (m, 1H), 3.96 (s, 2H), 3.54-3.45 (m, 2H), 3.21 (t, J=7.2 Hz, 2H), 2.83 (q, J=7.2 Hz, 2H), 2.25-2.07 (m, 2H), 1.21 (d, J=2.4 Hz, 12H).

### Methyl 4-chloro-2-(1-(3-(thiazol-2-yl)propanoyl)-1,2,5,6-tetrahydropyridin-3-yl)benzo[d]thiazole-6-carboxylate (Intermediate CD)

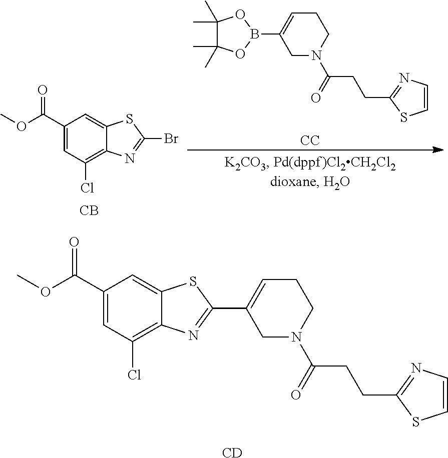

A mixture of methyl 2-bromo-4-chlorobenzo[d]thiazole-6-carboxylate (293 mg, 689 μmol, Intermediate CB). 1-(5-(4,4,5,5-tetramethyl-1,3,2-dioxaborolan-2-yl)-3,6-dihydropyridin-1(2H)-yl)-3-(thiazol-2-yl)propan-1-one (240 mg, 689 μmol, Intermediate CC), K₂CO₃ (285 mg, 2.07 mmol) and Pd(dppf)Cl₂ (50.4 mg, 68.9 μmol) in dioxane (4 mL) and H₂O (1 mL) was degassed and purged with N₂ three times. Then the mixture was stirred at 80° C. for 2 hrs under N₂ atmosphere. On completion, the mixture was triturated with H₂O (10 mL) at 0° C. for 15 min, then filtered to give the title compound (300 mg) as a white solid. LC-MS (ESI⁺) m/z 447.9 (M+H)⁺.

### 4-(2-Methoxy-4-(piperazin-1-yl)phenyl)-N,N-dimethyl-2-(1-(3-(thiazol-2-yl)propanoyl)-1,2,5,6-tetrahydropyridin-3-yl)benzo[d]thiazole-6-carboxamide (Intermediate CE)

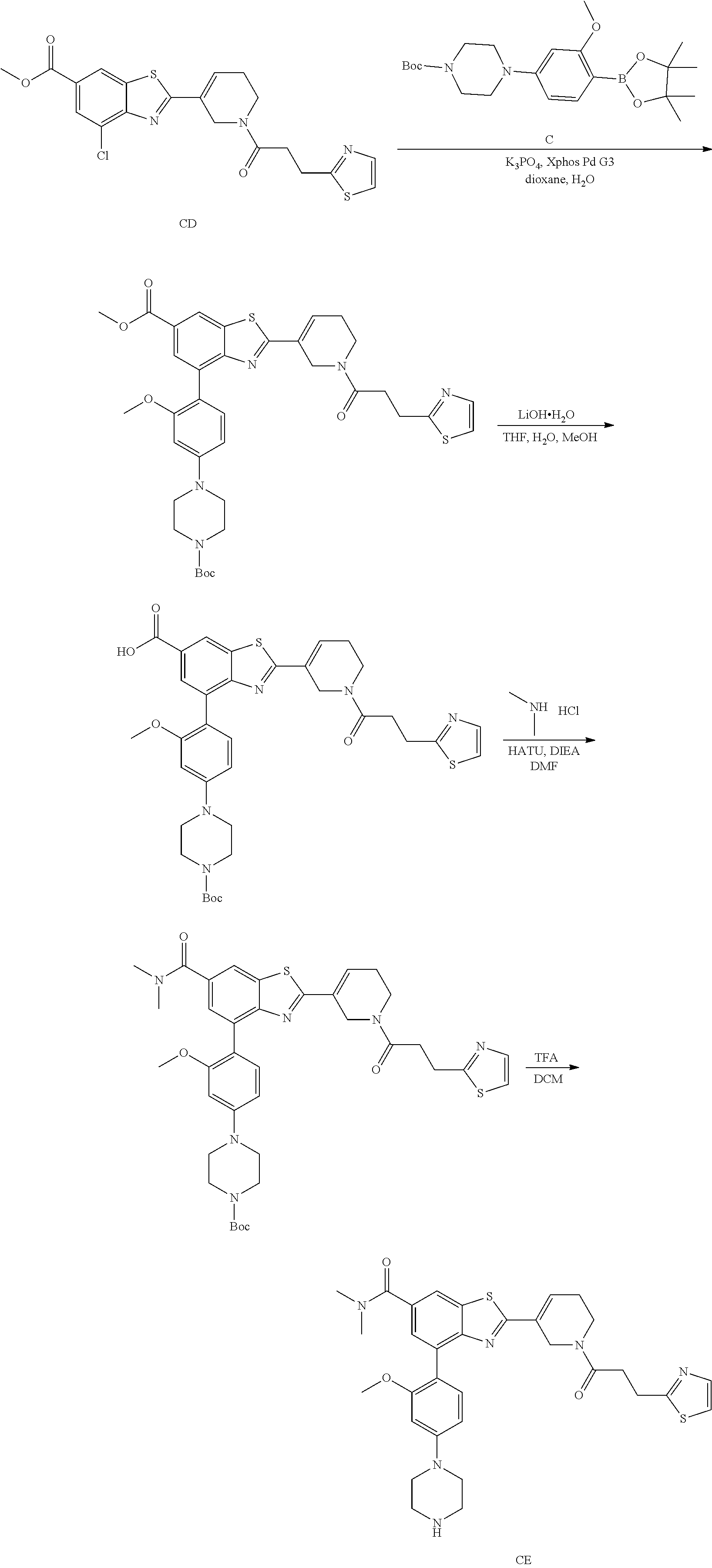

### Step 1—Methyl 4-(4-(4-(tert-butoxycarbonyl)piperazin-1-yl)-2-methoxyphenyl)-2-(1-(3-(thiazol-2-yl)propanoyl)-1,2,5,6-tetrahydropyridin-3-yl)benzo[d]thiazole-6-carboxylate

A mixture of methyl 4-chloro-2-(1-(3-(thiazol-2-yl)propanoyl)-1,2,5,6-tetrahydropyridin-3-yl)benzo[d]thiazole-6-carboxylate (160 mg, 357 μmol, Intermediate CD), tert-butyl 4-(3-methoxy-4-(4,4,5,5-tetramethyl-1,3,2-dioxaborolan-2-yl)phenyl)piperazine-1-carboxylate (149 mg, 357 μmol, Intermediate D). K₃PO₄ (227 mg, 1.07 mmol), and XPhos Pd G3 (30.2 mg, 35.7 μmol) in dioxane (2 mL) and H₂O (0.5 mL) was degassed and purged with N₂ three times. Then the mixture was stirred at 80° C. for 2 hrs under N₂ atmosphere. On completion, the mixture was triturated with H₂O (10 mL) at 0° C. for 15 min, then filtered to give the title compound (250 mg) as a white solid. LC-MS (ESI⁺) m/z 704.3 (M+H)⁺.

### Step 2—4-(4-(4-(Tert-butoxycarbonyl)piperazin-1-yl)-2-methoxyphenyl)-2-(1-(3-(thiazol-2-yl)propanoyl)-1,2,5,6-tetrahydropyridin-3-yl)benzo[d]thiazole-6-carboxylic acid

To a solution of methyl 4-(4-(4-(tert-butoxycarbonyl)piperazin-1-yl)-2-methoxyphenyl)-2-(1-(3-(thiazol-2-yl)propanoyl)-1,2,5,6-tetrahydropyridin-3-yl)benzo[d]thiazole-6-carboxylate (250 mg, 355 μmol) in H₂O (0.5 mL), MeOH (0.5 mL), and THF (2 mL) was added LiOH·H₂O (37.2 mg, 887 μmol). On completion, the mixture was stirred at 80° C. for 2 hrs. On completion, the mixture was concentrated under reduced pressure. The mixture was concentrated under reduced pressure to give the title compound (230 mg) as a white solid. LC-MS (ESI⁺) m/z 690.2 (M+H)⁺.

### Step 3—Tert-butyl 4-(4-(6-(dimethylcarbamoyl)-2-(1-(3-(thiazol-2-yl)propanoyl)-1,2,5,6-tetrahydropyridin-3-yl)benzo[d]thiazol-4-yl)-3-methoxyphenyl)piperazine-1-carboxylate

To a solution of 4-(4-(4-(tert-butoxycarbonyl)piperazin-1-yl)-2-methoxyphenyl)-2-(1-(3-(thiazol-2-yl)propanoyl)-1,2,5,6-tetrahydropyridin-3-yl)benzo[d]thiazole-6-carboxylic acid (200 mg, 289 mol) in DMF (2 mL) was added HATU (165 mg, 434 μmol), DIEA (187 mg, 1.45 mmol, 252 μL), and N-methylmethanamine (47 mg, 579 μmol, HCl, CAS #124-40-3). The mixture was then stirred at 25° C. for 2 hrs. On completion, the mixture was triturated with H₂O at 0° C. for 15 min, then filtered to give the title compound (200 mg) as a white solid. LC-MS (ESI⁺) m/z 717.3 (M+H)⁺.

### Step 4—4-(2-Methoxy-4-(piperazin-1-yl)phenyl)-N,N-dimethyl-2-(1-(3-(thiazol-2-yl)propanoyl)-1,2,5,6-tetrahydropyridin-3-yl)benzo[d]thiazole-6-carboxamide

To a solution of tert-butyl 4-(4-(6-(dimethylcarbamoyl)-2-(1-(3-(thiazol-2-yl)propanoyl)-1,2,5,6-tetrahydropyridin-3-yl)benzo[d]thiazol-4-yl)-3-methoxyphenyl)piperazine-1-carboxylate (200 mg, 278 μmol) in DCM (2 mL) was added TFA (31.8 mg, 278 μmol, 20.7 μL). The mixture was then stirred at 25° C. for 2 hrs. On completion, the mixture was concentrated under reduced pressure to give the title compound (150 mg, TFA) as a blue solid. LC-MS (ESI⁺) m/z 617.2 (M+H)⁺.

### (R)-1-(3-(7-fluoro-4-(4-(piperazin-1-yl)phenyl)-2-(pyrrolidine-1-carbonyl)-1H-indol-6-yl)piperidin-1-yl)-3-(1H-pyrazol-1-yl)propan-1-one (Intermediate CF)

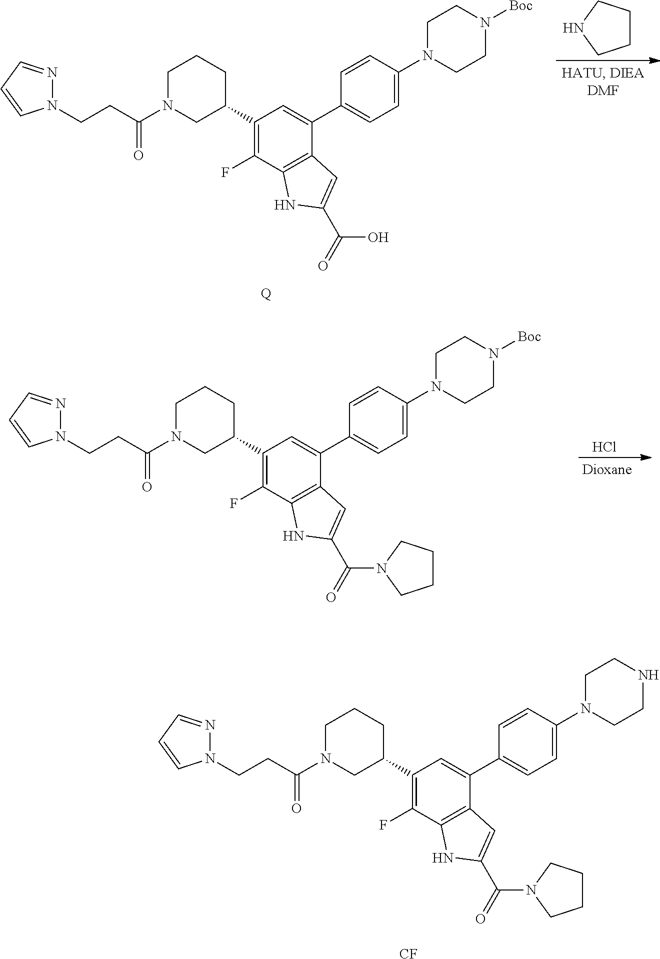

### Step 1—Tert-butyl (R)-4-(4-(6-(1-(3-(1H-pyrazol-1-yl)propanoyl)piperidin-3-yl)-7-fluoro-2-(pyrrolidine-1-carbonyl)-1H-indol-4-yl)phenyl)piperazine-1-carboxylate

To a solution of (R)-6-(1-(3-(1H-pyrazol-1-yl)propanoyl)piperidin-3-yl)-4-(4-(4-(tert-butoxycarbonyl)piperazin-1-yl)phenyl)-7-fluoro-1H-indole-2-carboxylic acid (400 mg, 620 μmol, Intermediate Q) in DMF (4 mL) was added HATU (283 mg, 744 μmol), HOBt (125 mg, 930 mol), DIEA (400 mg, 3.10 mmol, 540 L) and pyrrolidine (110 mg, 1.55 mmol, 129 μL, CAS #123-75-1). The mixture was then stirred at 25° C. for 2 hrs. On completion, the mixture was triturated with H₂O at 0 C for 15 min, then filtered to give the title compound (420 mg) as a white solid. LC-MS (ESI⁺) m/z 698.4 (M+H)⁺.

### Step 2—(R)-1-(3-(7-fluoro-4-(4-(piperazin-1-yl)phenyl)-2-(pyrrolidine-1-carbonyl)-1H-indol-6-yl)piperidin-1-yl)-3-(1H-pyrazol-1-yl)propan-1-one

To a solution of tert-butyl (R)-4-(4-(6-(1-(3-(1H-pyrazol-1-yl)propanoyl)piperidin-3-yl)-7-fluoro-2-(pyrrolidine-1-carbonyl)-1H-indol-4-yl)phenyl)piperazine-1-carboxylate (340 mg, 487 mol) in DCM (4 mL) was added HCl/dioxane (4 M, 121 μL). The mixture was then stirred at 25° C. for 2 hrs. On completion, the mixture was concentrated under reduced pressure to give the title compound (270 mg, HCl) as a red solid. LC-MS (ESI⁺) n/z 598.2 (M+H)f.

### 5-Bromo-7-chloro-4-fluoro-N,N-dimethylbenzofuran-2-carboxamide (Intermediate CG)

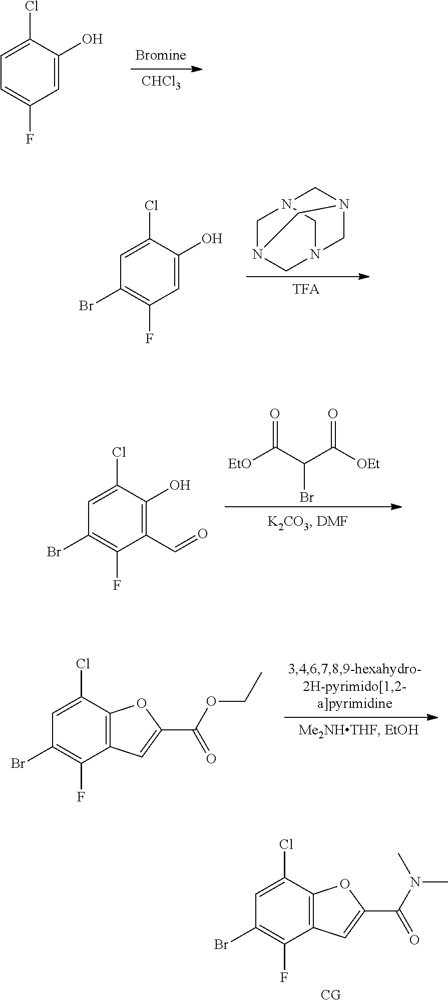

### Step 1—4-Bromo-2-chloro-5-fluorophenol

To a solution of 2-chloro-5-fluorophenol (50 g, 341 mmol, CAS #3827-49-4) in CHCl₃ (500 mL) was dropwise added Br₂ (54.5 g, 341 mmol, 17.5 mL) in CHCl₃ (82 mL) at 75° C., and the mixture was stirred at 75° C. for 3 h. Then Br₂ (10.9 g, 68.2 mmol, 3.52 mL) in CHCl₃ (31 mL) was added, and the mixture was stirred at 75° C. for 1 hr. On completion, the reaction mixture was quenched by addition of Na₂SO₃ solution (500 mL) at 0° C., and then extracted with DCM (900 mL). The combined organic layers were dried over Na₂SO₄, filtered and the filtrate was concentrated under reduced pressure to give a residue. The residue was purified by flash silica gel chromatography (ISCO®; 3000 g SepaFlash® Silica Flash Column, Eluent of 0˜1% Ethyl acetate/Petroleum ethergradient @100 mL/min) to give the title compound (25 g, 27% yield) as a yellow oil. LC-MS (ESI⁺) m/z 222.8 (M−H)⁺.

### Step 2—3-Bromo-5-chloro-2-fluoro-6-hydroxybenzaldehyde

A solution of 4-bromo-2-chloro-5-fluorophenol (22 g, 97.5 mmol) in TFA (200 mL) was degassed and purged with nitrogen before cooling to 0° C. Then 1,3,5,7-tetraazaadamantane (15.0 g, 107 mmol, CAS #100-97-0) was added to the mixture at 0° C. The mixture was stirred at 20° C. for 0.5 h, then stirred at 80° C. for 12 h. On completion, water (50 mL) was added at 0° C. followed by 50% H₂SO₄ (50 mL). The mixture was stirred for 1 hr and filtered, then diluted with water (500 mL) and extracted with EA (300 mL×3). The combined organic layers were washed with brine (300 mL×2), dried over Na₂SO₄, filtered and concentrated under reduced pressure to give a residue. The residue was purified by flash silica gel chromatography (ISCO®; 220 g SepaFlash® Silica Flash Column, Eluent of 0˜1% Ethyl acetate/Petroleum ethergradient @100 mL/min) to give the title compound (6 g, 23% yield) as a yellow oil. LC-MS (ESI⁺) m/z 250.7 (M−H)⁺.

### Step 3—Ethyl 5-bromo-7-chloro-4-fluorobenzofuran-2-carboxylate

To a solution of 3-bromo-5-chloro-2-fluoro-6-hydroxybenzaldehyde (6 g, 23.7 mmol) and diethyl 2-bromomalonate (11.3 g, 47.3 mmol, 8.07 mL, CAS #685-87-0) in DMF (60 mL) was added K₂CO₃ (6.54 g, 47.3 mmol), then the mixture was stirred at 90° C. for 12 h. On completion, the reaction mixture was diluted with water (500 mL) and extracted with EtOAc (200 mL×3). The combined organic layers were washed with brine (300 mL×2), dried over Na₂SO₄, filtered and concentrated under reduced pressure to give a residue. The residue was purified by flash silica gel chromatography (ISCO®; 80 g SepaFlash® Silica Flash Column, Eluent of 0˜1% Ethyl acetate/Petroleum ethergradient @ 50 mL/min) to give the title compound (7 g, 92% yield) as a white solid. ¹H NMR (400 MHz, CDCl₃) δ=7.77-7.50 (m, 2H), 4.46 (q, 7.2, 2H), 1.44 (t, 7.2, 3H).

### Step 4—5-Bromo-7-chloro-4-fluoro-N,N-dimethylbenzofuran-2-carboxamide

To a solution of ethyl 5-bromo-7-chloro-4-fluorobenzofuran-2-carboxylate (7 g, 21.7 mmol) and N-methylmethanamine (2 M, 16.3 mL, THF) in EtOH (70 mL) was added 3,4,6,7,8,9-hexahydro-2H-pyrimido[1,2-a]pyrimidine (909 mg, 6.53 mmol), then the mixture was stirred at 20° C. for 1 hr. On completion, the reaction mixture was diluted with water (300 mL) and extracted with DCM (50 mL×3). The combined organic layers were washed with brine (50 mL×2), dried over Na₂SO₄, filtered and concentrated under reduced pressure to give the title compound (6.7 g, 91% yield) as a white solid. LC-MS (ESI⁺) m/z 319.9 (M+H)⁺.

### 5-(1-(3-(1H-pyrazol-1-yl)propanoyl)-1,2,5,6-tetrahydropyridin-3-yl)-7-chloro-4-fluoro-N,N-dimethylbenzofuran-2-carboxamide (Intermediate CH)

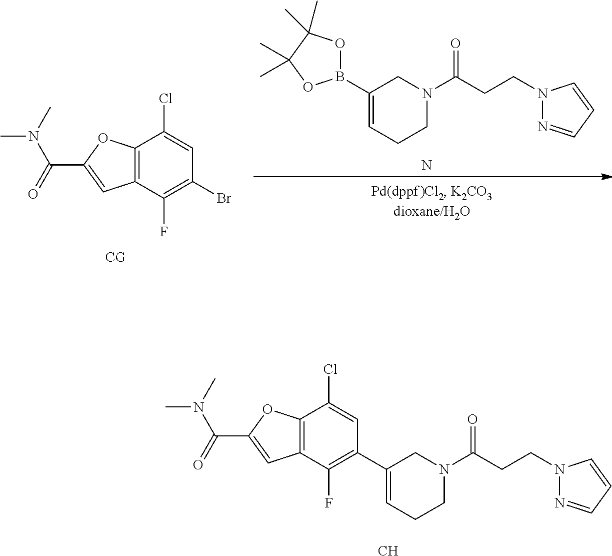

To a solution of 5-bromo-7-chloro-4-fluoro-N,N-dimethylbenzofuran-2-carboxamide (1.50 g, 4.68 mmol, Intermediate CG) and 3-(1H-pyrazol-1-yl)-1-(5-(4,4,5,5-tetramethyl-1,3,2-dioxaborolan-2-yl)- 3,6-dihydropyridin-1(2H)-yl)propan-1-one (1.70 g, 5.15 mmol, Intermediate N) in dioxane (15.0 mL) and H₂O (1.50 mL) was added Pd(dppf)Cl₂ (342 mg, 468 μmol) and K₂CO₃ (1.94 g, 14.0 mmol). Then the mixture was stirred at 80° C. for 2 hr under N₂ atmosphere. On completion, the reaction mixture was diluted with water (100 mL) and extracted with EA (30 mL×3). The combined organic layers were washed with brine (30 mL×2), dried over Na₂SO₄, filtered and the filtrate was concentrated under reduced pressure to give a residue. The residue was purified by flash silica gel chromatography (ISCO®; 20 g SepaFlash® Silica Flash Column, Eluent of 0˜50% Ethyl acetate/Petroleum ether gradient @20 mL/min) to afford the title compound (1.70 g, 81% yield) as a yellow gum. LC-MS (ESI⁺) m/z 445.1 (M+H)⁺.

### Tert-butyl 4-(3-chloro-4-(4,4,5,5-tetramethyl-1,3,2-dioxaborolan-2-yl)phenyl)piperazine-1-carboxylate (Intermediate CI)

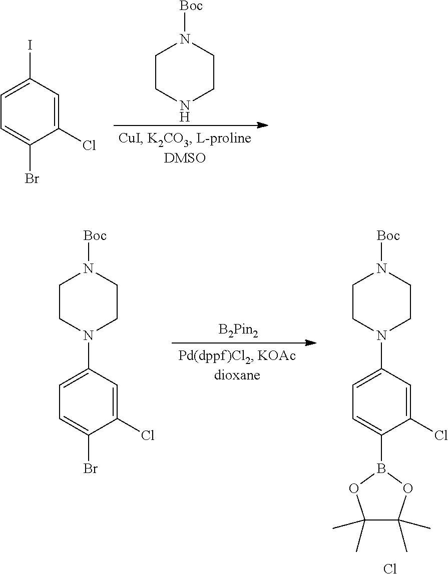 [SMILES: Clc1cc(I)ccc1Br ; CC(C)(C)OC(=O)N1CCNCC1 ; CC(C)(C)OC(=O)N1CCN(c2ccc(Br)c(Cl)c2)CC1 ; CC(C)(C)OC(=O)N1CCN(c2ccc(B3OC(C)(C)C(C)(C)O3)c(Cl)c2)CC1 ; Cl]

### Step 1—Tert-butyl 4-(4-bromo-3-chlorophenyl)piperazine-1-carboxylate

A mixture of tert-butyl piperazine-1-carboxylate (32.3 g, 145 mmol, CAS #57260-71-6), 1-bromo-2-chloro-4-iodobenzene (23.0 g, 72.5 mmol, CAS #535934-25-9), CuI (2.76 g, 14.5 mmol), K₂CO₃ (30.0 g, 217 mmol) and L-proline (1.67 g, 14.5 mmol) in DMSO (230 mL) was degassed and purged with N₂ three times. Then the mixture was stirred at 80° C. for 14 h under N₂ atmosphere. The reaction mixture was filtered and then diluted with water (1000 mL) and extracted with EA (1000 mL×3). The combined organic layers were washed with brine (1000 mL×3) and concentrated under reduced pressure to give a residue. The residue was purified by flash silica gel chromatography (ISCO®; 80 g SepaFlash® Silica Flash Column, Eluent of 0˜7% Ethyl acetate/Petroleum ether gradient @, 80 mL/min) to afford the title compound (22.7 g, 83% yield) as a white solid. ¹H NMR (400 MHz, DMSO-d₆) δ ppm 7.51 (d, J=8.8 Hz, 1H), 7.14 (d, J=2.8 Hz, 1H), 6.86 (dd, J=2.8, 9.2 Hz, 1H), 3.46-3.38 (m, 4H), 3.18-3.11 (m, 4H), 1.41 (s, 9H).

### Step 2—Tert-butyl 4-(3-chloro-4-(4,4,5,5-tetramethyl-1,3,2-dioxaborolan-2-yl)phenyl)piperazine-1-carboxylate

A mixture of tert-butyl 4-(4-bromo-3-chlorophenyl)piperazine-1-carboxylate (7.50 g, 19.9 mmol), B₂Pin₂ (5.58 g, 21.9 mmol), Pd(dppf)Cl₂ (1.46 g, 2.00 mmol) and KOAc (5.88 g, 59.9 mmol) in anhydrous dioxane (75.0 mL) was degassed and purged with N₂ three times. Then the mixture was stirred at 80° C. for 12 h under N₂ atmosphere. On completion, the reaction mixture was diluted with EtOAc (20.0 mL) filtered and concentrated under reduced pressure to give a residue. The residue was purified by flash silica gel chromatography (ISCO®; 220 g SepaFlash® Silica Flash Column, Eluent of 0˜15% Ethyl acetate/Petroleum ether gradient @ 100 mL/min) to afford the title compound (3.80 g, 44% yield) as a white solid. LC-MS (ESI⁺) m/z 423.2 (M+H)⁺; ¹H NMR (400 MHz, DMSO-d₆) δ ppm 7.49 (d, J=8.4 Hz, 1H), 6.91-6.80 (m, 2H), 3.42 (d, J=4.4 Hz, 4H), 3.22 (s, 4H), 1.42 (s, 9H), 1.27 (s, 12H).

### (S)-5-(1-(3-(1H-pyrazol-1-yl)propanoyl)piperidin-3-yl)-7-(2-chloro-4-(piperazin-1-yl)phenyl)-4-fluoro-N,N-dimethylbenzofuran-2-carboxamide (Intermediate CJ) and (R)-5-(1-(3-(1H-pyrazol-1-yl)propanoyl)piperidin-3-yl)-7-(2-chloro-4-(piperazin-1-yl)phenyl)-4-fluoro-N,N-dimethylbenzofuran-2-carboxamide (Intermediate CK)

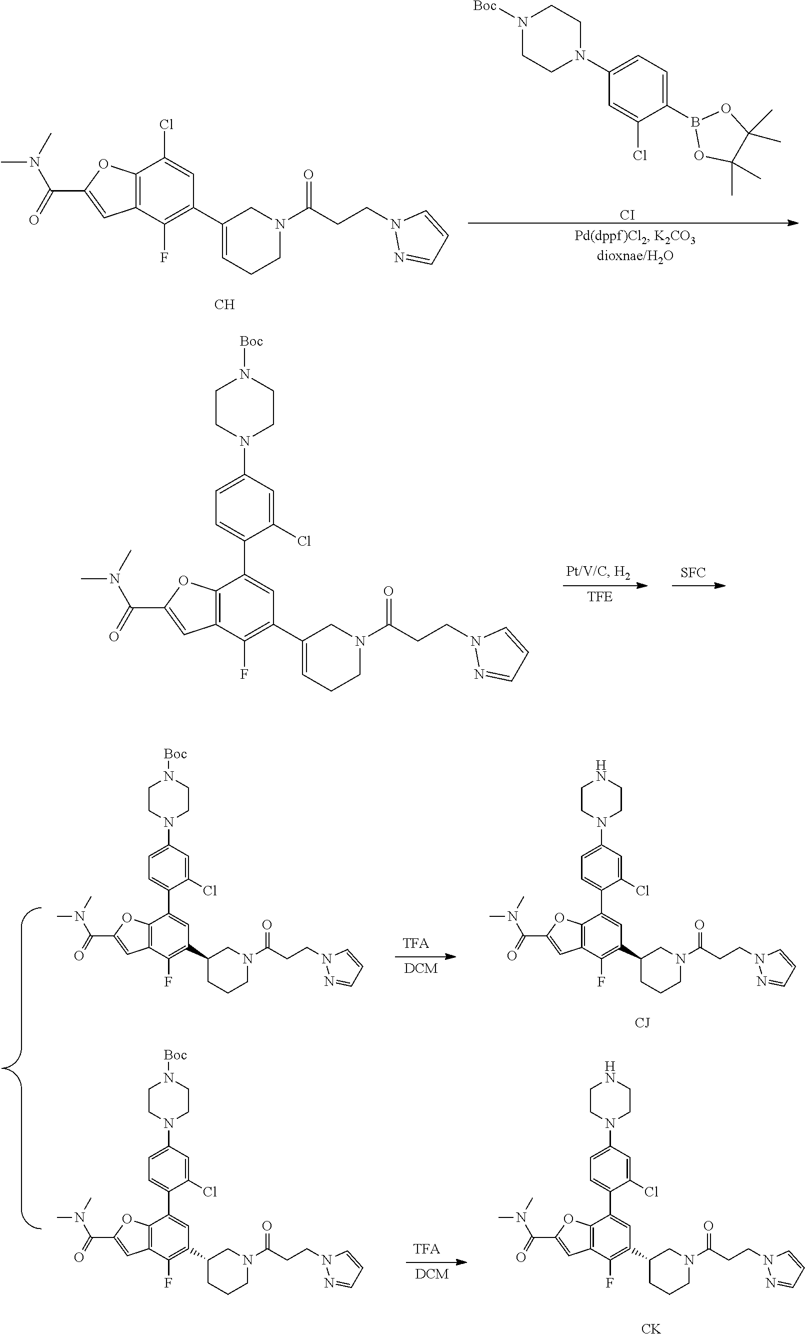

### Step 1—Tert-butyl 4-(4-(5-(1-(3-(1H-pyrazol-1-yl)propanoyl)-1,2,5,6-tetrahydropyridin-3-yl)-2-(dimethylcarbamoyl)-4-fluorobenzofuran-7-yl)-3-chlorophenyl)piperazine-1-carboxylate

To a solution of 5-(1-(3-(1H-pyrazol-1-yl)propanoyl)-1,2,5,6-tetrahydropyridin-3-yl)-7-chloro- 4-fluoro-N,N-dimethylbenzofuran-2-carboxamide (1.50 g, 3.37 mmol, Intermediate CH) and tert-butyl 4-(3-chloro-4-(4,4,5,5-tetramethyl-1,3,2-dioxaborolan-2-yl)phenyl)piperazine-1-carboxylate (1.43 g, 3.37 mmol, Intermediate CI) in dioxane (15.0 mL) and H₂O (1.50 mL) was added XPhos Pd G3 (285 mg, 337 mol) and K₃PO₄ (2.15 g, 10.1 mmol). Then the mixture was stirred at 80° C. for 2 h under N₂ atmosphere. On completion, the reaction mixture was diluted with water (100 mL) and extracted with EA (30 mL×3). The combined organic layers were washed with brine (30 mL×2), dried over Na₂SO₄, filtered and concentrated under reduced pressure to give a residue. The residue was purified by flash silica gel chromatography (ISCO®; 20 g SepaFlash® Silica Flash Column, Eluent of 30˜100% Ethyl acetate/Petroleum ether gradient a 20 mL/min) to afford the title compound (1.80 g, 69% yield) as a yellow gum. LC-MS (ESI⁺) m/z 705.3 (M+H)⁺.

### Step 2—Tert-butyl (S)-4-(4-(5-(1-(3-(1H-pyrazol-1-yl)propanoyl)piperidin-3-yl)-2-(dimethylcarbamoyl)-4-fluorobenzofuran-7-yl)-3-chlorophenyl)piperazine-1-carboxylate (4-P1) & tert-butyl (R)-4-(4-(5-(1-(3-(1H-pyrazol-1-yl)propanoyl)piperidin-3-yl)-2-(dimethylcarbamoyl)-4-fluorobenzofuran-7-yl)-3-chlorophenyl)piperazine-1-carboxylate

To a solution of tert-butyl 4-(4-(5-(I-(3-(1H-pyrazol-1-yl)propanoyl)-1,2,5,6-tetrahydropyridin-3-yl) -2-(dimethylcarbamoyl)-4-fluorobenzofuran-7-yl)-3-chlorophenyl)piperazine-1-carboxylate (1.00 g, 1.42 mmol) in MeOH (60.0 mL) was added Pt—V/C (1.00 g, 3.83 mmol), then the mixture was stirred at 40° C. for 1 hr under H₂ (15 psi). On completion, the reaction mixture was filtered and concentrated under reduced pressure to give a residue. The residue was purified by prep-HPLC (column: Phenomenex Luna C18 150*25 mm*10 um; mobile phase: [water (FA)-ACN]; gradient: 56%-86% B over 10 min) to give desired compound as a white solid. Then the compound was further separated by SFC (column: REGIS (R,R)WHELK-01(250 mm*25 mm, 10 um): mobile phase: [CO₂-ACN/i-PrOH(0.1% NH₃H₂O)]; B %:70%, isocratic elution mode) to give tert-butyl (S)-4-(4-(5-(1-(3-(1H-pyrazol-1-yl)propanoyl)piperidin-3-yl)-2-(dimethylcarbamoyl)-4-fluorobenzofuran-7-yl)-3-chlorophenyl)piperazine-1-carboxylate (274 mg, 27% yield) as a white gum (LC-MS (ESI⁺) m/z 702.2 (M+H)⁺) and tert-butyl (R)-4-(4-(5-(1-(3-(1H-pyrazol-1-yl)propanoyl)piperidin-3-yl)-2-(dimethylcarbamoyl)-4-fluorobenzofuran-7-yl)-3-chlorophenyl)piperazine-1-carboxylate (236 mg, 23% yield) as a white gum (LC-MS (ESI⁺) m/z 702.3 (M+H)⁺). The absolute stereochemistry of the enantiomers was assigned arbitrarily.

### Step 3—(S)-5-(1-(3-(1H-pyrazol-1-yl)propanoyl)piperidin-3-yl)-7-(2-chloro-4-(piperazin-1-yl)phenyl)-4-fluoro-N,N-dimethylbenzofuran-2-carboxamide

To a solution of tert-butyl (S)-4-(4-(5-(1-(3-(1H-pyrazol-1-yl)propanoyl)piperidin-3-yl)-2-(dimethylcarbamoyl)-4-fluorobenzofuran-7-yl)-3-chlorophenyl)piperazine-1-carboxylate (274 mg, 387 μmol) in DCM (3.00 mL) was added TFA (921 mg, 8.08 mmol), then the mixture was stirred at 20° C. for 1 h. On completion, the reaction mixture was filtered and concentrated under reduced pressure to give the title compound (696 mg, 94% yield, TFA) as a brown gum. LC-MS (ESI⁺) m/z 607.2 (M+H)⁺.

### Step 4—(R)-5-(1-(3-(1H-pyrazol-1-yl)propanoyl)piperidin-3-yl)-7-(2-chloro-4-(piperazin-1-yl)phenyl)-4-fluoro-N,N-dimethylbenzofuran-2-carboxamide

To a solution of tert-butyl (R)-4-(4-(5-(1-(3-(1H-pyrazol-1-yl)propanoyl)piperidin-3-yl)-2-(dimethylcarbamoyl)-4-fluorobenzofuran-7-yl)-3-chlorophenyl)piperazine-1-carboxylate (236 mg, 334 μmol) in DCM (3.00 mL) was added TFA (38.0 mg, 334 μmol), then the mixture was stirred at 20° C. for 1 hr. On completion, the reaction mixture was filtered and concentrated under reduced pressure to give the title compound (564 mg, 94% yield, TFA) as a brown gum. LC-MS (ESI⁺) m/z 607.2 (M+H)⁺.

### 7-Chloro-N,N-dimethyl-2-(1,2,5,6-tetrahydropyridin-3-yl)-1H-indole-5-carboxamide (Intermediate CL)

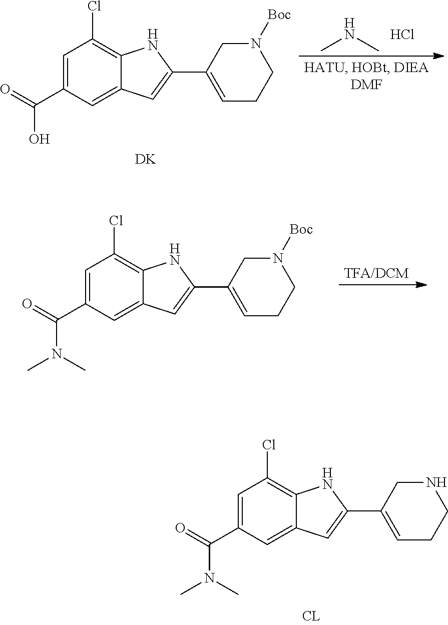

### Step 1—Tert-butyl 5-(7-chloro-5-(dimethylcarbamoyl)-1H-indol-2-yl)-3, 6-dihydropyridine-1(2H)-carboxylate

A solution of 2-(1-(tert-butoxycarbonyl)-1,2,5,6-tetrahydropyridin-3-yl)-7-chloro-1H-indole-5-carboxylic acid (1.80 g, 3.82 mmol. Intermediate DK), dimethylamine hydrochloride (935 mg, 11.5 mmol, CAS #506-59-2). HATU (1.74 g, 4.59 mmol), HOBt (1.03 g, 7.64 mmol) and DIEA (2.47 g, 19.1 mmol, 3.33 mL) in DMF (5 mL) was stirred at 25° C. for 2 hrs. On completion, the mixture was triturated with water at 25° C. for 12 hrs. Then the mixture was filtered and the filter cake was concentrated in vacuo to give the title compound (2.0 g) as a white solid. LC-MS (ESI⁺) m/z 404.2 (M+H)⁺.

### Step 2—7-Chloro-N,N-dimethyl-2-(1,2,5,6-tetrahydropyridin-3-yl)-1H-indole-5-carboxamide

A solution of tert-butyl 5-(7-chloro-5-(dimethylcarbamoyl)-1H-indol-2-yl)-3,6-dihydropyridine-1(2H)-carboxylate (1.20 g, 2.97 mmol) in DCM (2 mL) and TFA (2 mL) was stirred at 25° C. for 2 hrs. On completion, the residue was concentrated in vacuo to give the title compound (900 mg) as a yellow solid. LC-MS (ESI⁺) m/z 304.0 (M+H)⁺.

### 2-(1-(3-(1H-1,2,3-triazol-1-yl)propanoyl)-1,2,5,6-tetrahydropyridin-3-yl)-7-chloro-N,N-dimethyl-1H-indole-5-carboxamide (Intermediate CM)

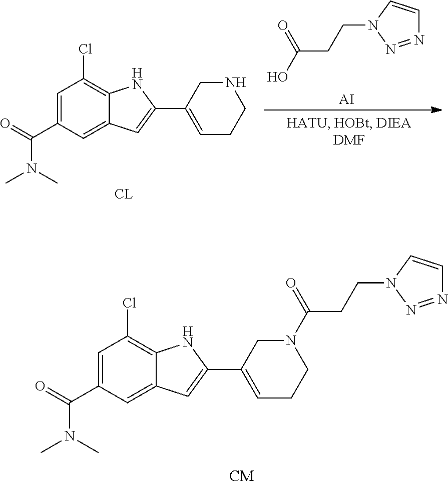 [SMILES: CN(C)C(=O)c1cc(Cl)c2[nH]c(C3=CCCNC3)cc2c1 ; O=C(O)CCn1ccnn1 ; "AI" ; CN(C)C(=O)c1cc(Cl)c2[nH]c(C3=CCCN(C(=O)CCn4ccnn4)C3)cc2c1]

A solution of 7-chloro-N,N-dimethyl-2-(1,2,5,6-tetrahydropyridin-3-yl)-1H-indole-5-carboxamide (800 mg, 2.63 mmol, Intermediate CL), 3-(1H-1,2,3-triazol-1-yl)propanoic acid (1.49 g, 10.53 mmol, Intermediate AI), HOBt (712 mg, 5.27 mmol), HATU (1.20 g, 3.16 mmol) and DIEA (1.02 g, 7.90 mmol, 1.38 mL) in DMF (10 mL) was stirred at 25° C. for 2 hrs. On completion, the reaction was purified by reverse phase HPLC (0.8 g/L ammonium bicarbonate) to give the tittle compound (360 mg) as a white solid. LC-MS (ESI⁺) m/z 427.1 (M+H)⁺.

### 2-(1-(3-(1H-1,2,3-triazol-1-yl)propanoyl)-1,2,5,6-tetrahydropyridin-3-yl)-7-(2-methoxy-4-(piperazin-1-yl)phenyl)-N,N-dimethyl-1H-indole-5-carboxamide (Intermediate CN)

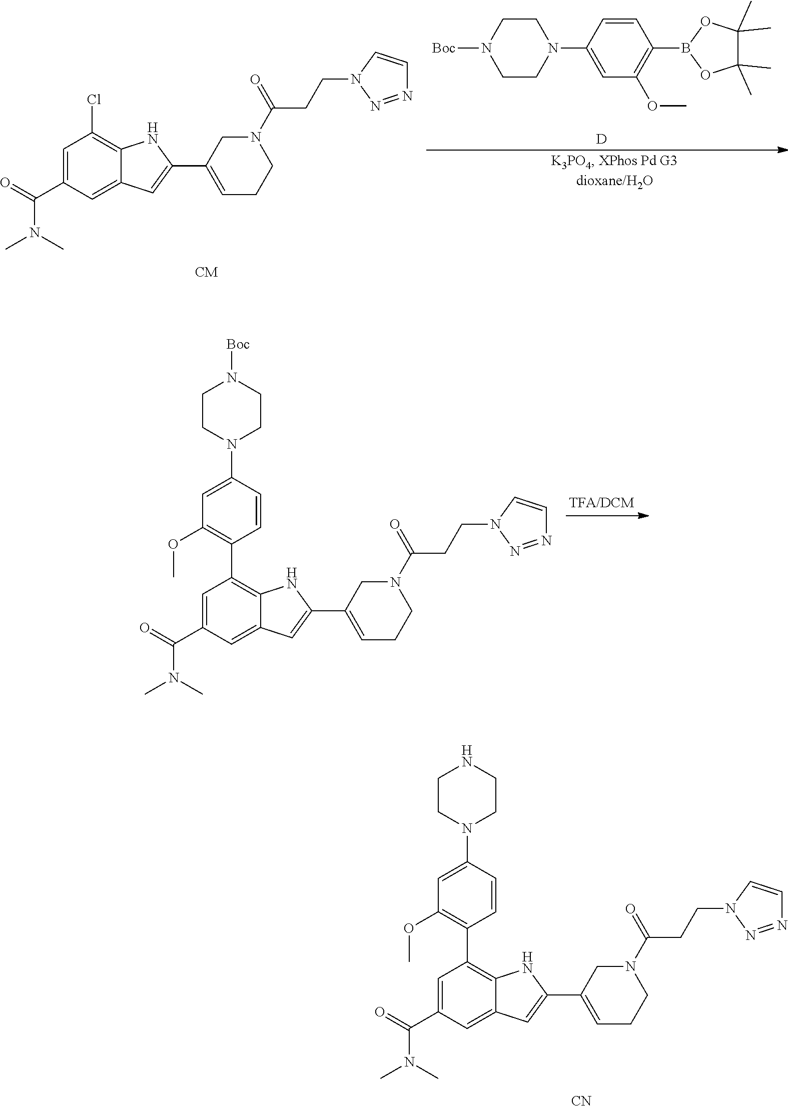

### Step 1—Tert-butyl 4-(4-(2-(1-(3-(1H-1,2,3-triazol-1-yl)propanoyl)-1,2,5,6-tetrahydropyridin-3-yl)-5-(dimethylcarbamoyl)-1H-indol-7-yl)-3-methoxyphenyl)piperazine-1-carboxylate

A solution of 2-(1-(3-(1H-1,2,3-triazol-1-yl)propanoyl)-1,2,5,6-tetrahydropyridin-3-yl)-7-chloro-N,N-dimethyl-1H-indole-5-carboxamide (70.0 mg, 164 gmol, Intermediate CM), tert-butyl 4-(3-methoxy-4-(4,4,5,5-tetramethyl-1,3,2-dioxaborolan-2-yl)phenyl)piperazine-1-carboxylate (82.3 mg, 197 gmol, Intermediate D), XPhos Pd G3 (27.8 mg, 32.8 gmol), and K₃PO₄ (104 mg, 492 gmol) in dioxane (1 mL) and H₂O (0.1 mL) was stirred at 80° C. for 2 hrs. On completion, the reaction was purified by column chromatography (SiO₂, Petroleum ether:Ethyl acetate=10/1 to 0/1) (SiO₂, Dichloromethane: Methanol=100/0 to 10/1) to give the tittle compound (100 mg, 84% yield) as a yellow solid. LC-MS (ESI⁺) m/z 683.4 (M+H)⁺.

### Step 2—2-(1-(3-(1H-1,2,3-triazol-1-yl)propanoyl)-1,2,5,6-tetrahydropyridin-3-yl)-7-(2-methoxy-4-(piperazin-1-yl)phenyl)-N,N-dimethyl-1H-indole-5-carboxamide

A solution of tert-butyl 4-(4-(2-(1-(3-(1H-1,2,3-triazol-1-yl)propanoyl)-1,2,5,6-tetrahydropyridin-3-yl)-5-(dimethylcarbamoyl)-1H-indol-7-yl)-3-methoxyphenyl)piperazine-1-carboxylate (120 mg, 176 μmol), in TFA (1 mL) and DCM (1 mL) was stirred at 25° C. for 2 hrs. On completion, the residue was concentrated in vacuo to give the tittle compound (100 mg) as a yellow solid. LC-MS (ESI⁺) m/z 583.3 (M+H)⁺.

### 4-chloro-N,N-dimethyl-2-(1-(3-(thiazol-2-yl)propanoyl)-1,2,5,6-tetrahydropyridin-3-yl)benzo[d]thiazole-6-carboxamide (Intermediate CO)

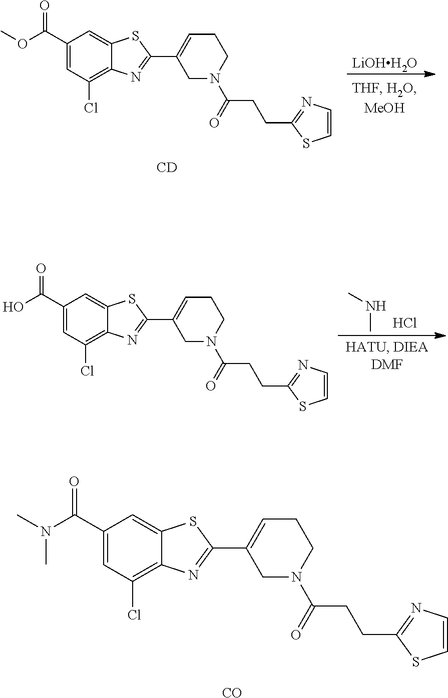

### Step 1—4-Chloro-2-(1-(3-(thiazol-2-yl)propanoyl)-1,2,5,6-tetrahydropyridin-3-yl)benzo[d]thiazole-6-carboxylic acid

To a solution of methyl 4-chloro-2-(1-(3-(thiazol-2-yl)propanoyl)-1,2,5,6-tetrahydropyridin-3-yl)benzo[d]thiazole-6-carboxylate (500 mg, 1.16 mmol, Intermediate CD) in THF (4 mL), H₂O (1 mL), MeOH (1 mL) was added LiOH·H₂O (83.3 mg, 3.48 mmol), then the reaction was stirred at 25° C. for 2 hrs. On completion, HCl (1N) was added to the reaction mixture until the pH=5, then diluted by water (10 mL) and extracted by ethyl acetate (4×20 mL). The combined organic layers were dried over anhydrous sodium sulfate, filtered and the filtrate was concentrated in vacuo to give the title compound (450 mg) as a yellow solid. LC-MS (ESI⁺) m/z 416.9 (M+H)⁺.

### Step 2—4-Chloro-N,N-dimethyl-2-(1-(3-(thiazol-2-yl)propanoyl)-1,2,5,6-tetrahydropyridin-3-yl)benzo[d]thiazole-6-carboxamide

To a solution of 4-chloro-2-(1-(3-(thiazol-2-yl)propanoyl)-1,2,5,6-tetrahydropyridin-3-yl)benzo[d]thiazole-6-carboxylic acid (450 mg, 1.08 mmol) in DMF (5 mL) was added HATU (615 mg, 1.62 mmol), DIEA (697 mg, 5.40 mmol, 940 L) and dimethylamine hydrochloride (176 mg, 2.16 mmol, CAS #506-59-2). The mixture was then stirred at 25° C. for 2 hrs. On completion, the reaction mixture was diluted with water (20 mL) and extracted with ethyl acetate (4×50 mL). The combined organic layers were washed with brine (20 mL) and dried over anhydrous sodium sulfate, filtered and the filtrate was concentrated in vacuo to give the crude residue The residue was purified by column chromatography (SiO₂, Dichloromethane:Methanol=1/0 to 20/1) to give the title compound (330 mg, 63% yield) as a red solid. LC-MS (ESI⁺) m/z443.8 (M+H)⁺.

### 4-Bromo-1-(4-(4,4,5,5-tetramethyl-1,3,2-dioxaborolan-2-yl)phenyl)piperidine (Intermediate CP)

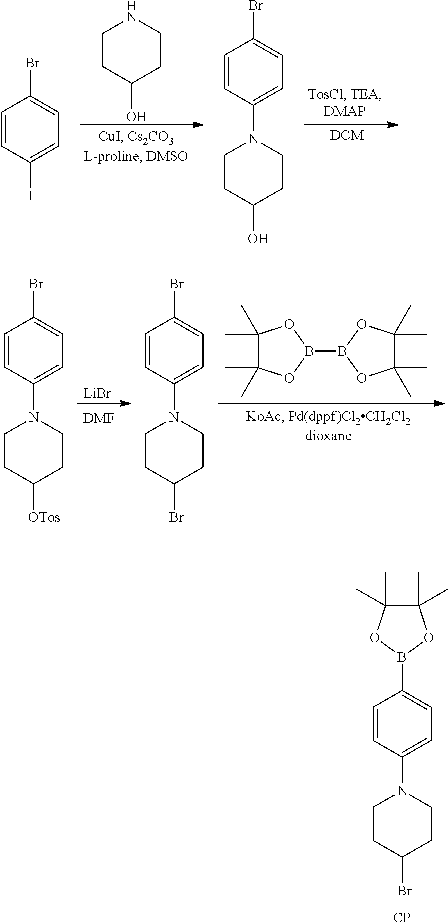

### Step 1—1-(4-Bromophenyl)piperidin-4-ol

To a solution of 1-bromo-4-iodobenzene (5 g, 17.6 mmol. CAS #589-87-7), piperidin-4-ol (1.79 g, 17.6 mmol, CAS #5382-16-1) in DMSO (60 mL) was added Cs₂CO₃ (14.4 g, 44.1 mmol), CuI (673 mg, 3.53 mmol), and L-proline (813 mg, 7.07 mmol). Then the reaction was stirred at 90° C. for 12 hrs under nitrogen atmosphere. On completion, the reaction mixture vas diluted with water (200 mL). Then the reaction mixture was filtered and the filter cake was dried in vacuo to give the title compound (3.5 g) as a yellow solid. LC-MS (ESI⁺) m/z 255.8 (M+H)⁺.

### Step 2—1-(4-Bromophenyl)piperidin-4-yl 4-methylbenzenesulfonate

To a solution of 1-(4-bromophenyl)piperidin-4-ol (1 g, 3.90 mmol) in DCM (10 mL) was added TEA (1.19 g, 11.7 mmol, 1.63 mL), DMAP (47.7 mg, 390 μmol) and TosCl (1.49 g, 7.81 mmol), then the reaction was stirred at 25° C. for 12 hrs. On completion, the reaction mixture was diluted with water (10 mL) and extracted with dichloromethane (4×50 mL). The combined organic layers were washed with brine (10 mL) and dried over anhydrous sodium sulfate, filtered and the filtrate was concentrated in vacuo to give the crude residue. The residue was purified by column chromatography (SiO₂, Petroleum ether:Ethyl acetate=1/0 to 10/1) to give the title compound (1.3 g, 75% yield) as a white solid. LC-MS (ESI⁺) m/z 409.7 (M+H)⁺.

### Step 2—4-Bromo-1-(4-bromophenyl)piperidine

To a solution of 1-(4-bromophenyl)piperidin-4-yl 4-methylbenzenesulfonate (1.2 g, 2.92 mmol) in DMF (12 mL) was added LiBr (761 mg, 8.77 mmol, 220 L), then the reaction was stirred at 50° C. for 12 hrs under nitrogen atmosphere. On completion, the reaction mixture was diluted with water (20 mL) and extracted with dichloromethane (4×50 mL). The combined organic layers were washed with brine (20 mL) and dried over anhydrous sodium sulfate, filtered and the filtrate was concentrated in vacuo to give the crude residue. The residue was purified by column chromatography (SiO₂, Petroleum ether:Ethyl acetate=1/0 to 10/1) to give the title compound (800 mg, 83% yield) as a white solid. LC-MS (ESI⁺) m/z 317.9 (M+H)⁺.

### Step 3—4-Bromo-1-(4-(4,4,5,5-tetramethyl-1,3,2-dioxaborolan-2-yl)phenyl)piperidine

To a solution of 4-bromo-1-(4-bromophenyl)piperidine (600 mg, 1.88 mmol), 4,4,5,5-tetramethyl-2-(4,4,5,5-tetramethyl-1,3,2-dioxaborolan-2-yl)-1,3,2-dioxaborolane (2.39 g, 9.40 mmol) in dioxane (10 mL) was added Pd(dppf)Cl₂·CH₂Cl₂ (153 mg, 188 mol) and KOAc (553 mg, 5.64 mmol), then the reaction was stirred at 80° C. for 12 hrs. On completion, the reaction mixture was diluted with water (20 mL) and extracted with ethyl acetate (4×50 mL). The combined organic layers were washed with brine (20 mL) and dried over anhydrous sodium sulfate, filtered and the filtrate was concentrated in vacuo to give the title compound (600 mg, 65% yield) as a white solid. LC-MS (ESI⁺) m/z 365.9 (M+H)⁺.

### 3-(3-Methyl-2-oxo-5-(1-(4-(4,4,5,5-tetramethyl-1,3,2-dioxaborolan-2-yl)phenyl)piperidin-4-yl)-2,3-dihydro-1H-benzo[d]imidazol-1-yl)piperidine-2,6-dione (Intermediate CQ)

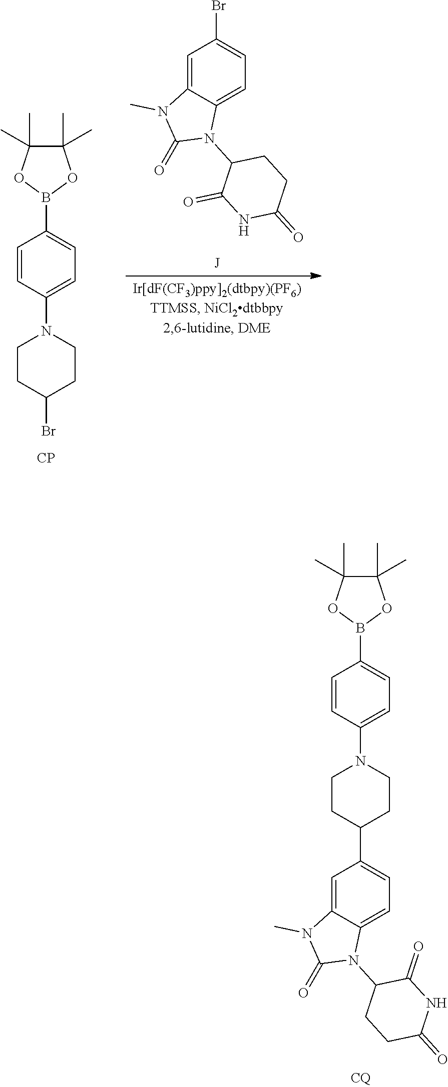

To a solution of 3-(5-bromo-3-methyl-2-oxo-2,3-dihydro-1H-benzo[d]imidazol-1-yl)piperidine-2,6-dione (400 mg, 1.18 mmol, Intermediate J), 4-bromo-1-(4-(4,4,5,5-tetramethyl-1,3,2-dioxaborolan-2-yl)phenyl)piperidine (562 mg, 1.54 mmol, Intermediate CP), Ir[dF(CF₃)ppy]₂(dtbpy)(PF₆) (13.2 mg, 11.8 μmol), NiCl₂·dtbbpy (7.06 mg, 17.7 mol), TTMSS (294 mg, 1.18 mmol, 364 μL), and 2,6-lutidine (1.14 g, 10.6 mmol, 1.24 mL) in DME (5 mL). The reaction was then stirred and irradiated with a 4×50 W [455 nm] blue LED lamp (3 cm away), with cooling water to keep the reaction temperature at 25° C. for 14 hrs. On completion, the reaction mixture was concentrated in vacuo to give the crude residue. The crude residue was purified by reverse phase HPLC (0.8 g/L ammonium bicarbonate) to give the title compound (70 mg, 9% yield) as a white solid. LC-MS (ESI⁺) m/z 545.1 (M+H)⁺.

### 2-(1-(3-(1H-1,2,3-triazol-1-yl)propanoyl)-1,2,5,6-tetrahydropyridin-3-yl)-N,N-dimethyl-7-(4-(piperazin-1-yl)phenyl)-1H-indole-5-carboxamide (Intermediate CR)

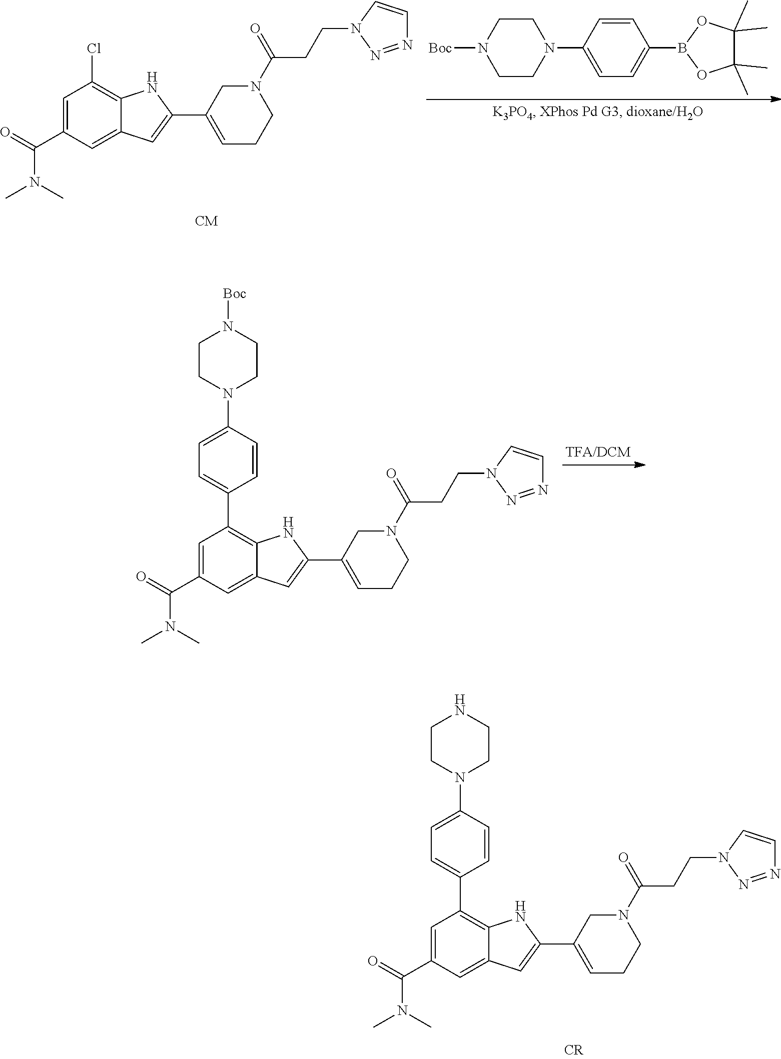

### Step 1—Tert-butyl 4-(4-(2-(1-(3-(1H-1,2,3-triazol-1-yl)propanoyl)-1,2,5,6-tetrahydropyridin-3-yl)-5-(dimethylcarbamoyl)-1H-indol-7-yl)phenyl)piperazine-1-carboxylate

A solution of 2-(1-(3-(1H-1,2,3-triazol-1-yl)propanoyl)-1,2,5,6-tetrahydropyridin-3-yl)-7-chloro-N,N-dimethyl-LH-indole-5-carboxamide (80.0 mg, 187 mol, Intermediate CM), tert-butyl 4-(4-(4,4,5,5-tetramethyl-1,3,2-dioxaborolan-2-yl)phenyl)piperazine-1-carboxylate (72.8 mg, 187 μmol, CAS #470478-90-1). K₃PO₄ (119 mg, 562 μmol), and XPhos Pd G3 (31.7 mg, 37.5 μmol) in dioxane (1 mL) and H₂O (0.5 mL) was stirred at 80° C. for 2 hrs. On completion, the reaction mixture was concentrated under reduced pressure to give a residue. The crude residue was purified by column chromatography (SiO₂. Petroleum ether/Ethyl acetate=1/0 to 0/1 to DCM/MeOH=10:1) to give the title compound (80.0 mg, 65% yield) as a yellow solid. LC-MS (ESI⁺) m/z 653.3 (M+H)⁺.

### Step 2—2-(1-(3-(1H-1,2,3-triazol-1-yl)propanoyl)-1,2,5,6-tetrahydropyridin-3-yl)-N,N-dimethyl-7-(4-(piperazin-1-yl)phenyl)-1H-indole-5-carboxamide

A solution of tert-butyl 4-(4-(2-(1-(3-(1H-1,2,3-triazol-1-yl)propanoyl)-1,2,5,6-tetrahydropyridin-3-yl)-5-(dimethylcarbamoyl)-1H-indol-7-yl)phenyl)piperazine-1-carboxylate (80.0 mg, 122 μmol) in DCM (1 mL) and TFA (1 mL) was stirred at 25° C. for 2 hrs. On completion, the mixture was concentrated in vacuum to give the title compound (80 mg, TFA) as a brown solid. LC-MS (ESI⁺) m/z 553.4 (M+H)⁺.

### 4-(2-Bromoethyl)-1-(4-(4,4,5,5-tetramethyl-1,3,2-dioxaborolan-2-yl)phenyl)piperidine (Intermediate CS)

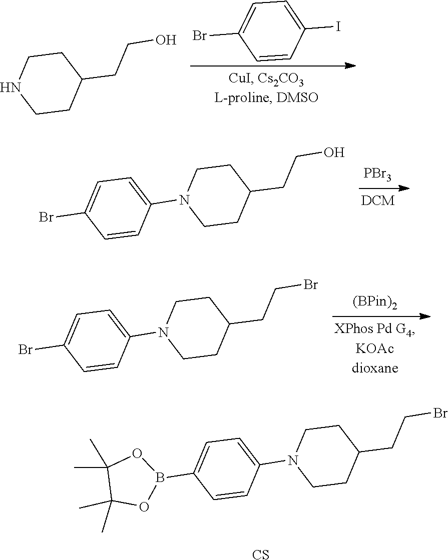

### Step 1—2-(1-(4-Bromophenyl)piperidin-4-yl)ethan-1-ol

A mixture of 2-(4-piperidyl)ethanol (5 g, 38.7 mmol, CAS #622-26-4),1-bromo-4-iodo-benzene (11.0 g, 38.7 mmol, CAS #589-87-7), Cs₂CO₃ (31.5 g, 96.8 mmol), CuI (1.47 g, 7.74 mmol) and L-proline (1.78 g, 15.5 mmol) in DMSO (200 mL) was degassed and purged with N₂ three times. Then the mixture was stirred at 90° C. for 12 hrs under N₂ atmosphere. On completion, the reaction mixture was filtered to give the filtrate. The filtrate was diluted with H₂O (100 mL) and extracted with EA (150 mL×3). The combined organic layers were washed with sat. NaCl (100 mL×3), dried over Na₂SO₄, filtered and concentrated under reduced pressure to give a residue. The residue was purified by prep-HPLC (0.1% FA condition) to give the title compound (5.4 g, 48% yield) as a black brown solid. LC-MS (ESI⁺) m/z 284.0 (M+H)⁺.

### Step 2—4-(2-Bromoethyl)-1-(4-bromophenyl)piperidine

To a solution of 2-[1-(4-bromophenyl)-4-piperidyl]ethanol (5 g, 17.6 mmol) in ACN (120 mL) was added PBr₃ (7.14 g, 26.4 mmol) at 0° C. The mixture was then stirred at 80° C. for 2 h. On completion, the reaction mixture was quenched by pouring into H₂O (300 mL) at 25° C., and then NH₃·H₂O was added until pH 7 and extracted with EA (150 mL×3). The combined organic layers were concentrated under reduced pressure to give the title compound (4.7 g) as a yellow solid. LC-MS (ESI⁺) m/z 347.9 (M+H)

### Step 3—4-(2-Bromoethyl)-1-(4-(4,4,5,5-tetramethyl-1,3,2-dioxaborolan-2-yl)phenyl)piperidine

A mixture of 4-(2-bromoethyl)-1-(4-bromophenyl)piperidine (4.3 g, 12 mmol), 4,4,5,5-tetramethyl-2-(4,4,5,5-tetramethyl-1,3,2-dioxaborolan-2-yl)-1,3,2-dioxaborolane (3.78 g, 14.9 mmol), KOAc (2.43 g, 24.8 mmol), and Xphos Pd G4 (1.07 g, 1.24 mmol) in dioxane (100 mL) was degassed and purged with N₂ three times. Then the mixture was stirred at 60° C. for 3 hrs under N₂ atmosphere. On completion, the reaction mixture was filtered and the filtrate was concentrated under reduced pressure to give a residue. The residue was purified by column chromatography (SiO₂, Petroleum ether:Ethyl acetate=1/0 to 24/1) to give the title compound (250 mg, 66% yield) as a yellow solid. LC-MS (ESI⁺) m/z 393.9 (M+H)⁺. ¹H NMR (400 MHz, DMSO-d₆) δ=7.48 (d, J=8.4 Hz, 2H), 6.88 (d, J=8.4 Hz, 2H), 3.79 (d, J=12.8 Hz, 2H), 3.59 (t, J=6.8 Hz, 2H), 2.70 (t, J=12.4 Hz, 2H), 1.81-1.68 (m, 4H), 1.68-1.58 (m, 1H), 1.25 (s, 12H), 1.21 (d, J=3.2 Hz, 2H).

### 3-(3-Methyl-2-oxo-4-(2-(1-(4-(4,4,5,5-tetramethyl-1,3,2-dioxaborolan-2-yl)phenyl)piperidin-4-yl)ethyl)-2,3-dihydro-1H-benzo[d]imidazol-1-yl)piperidine-2,6-dione (Intermediate CT)

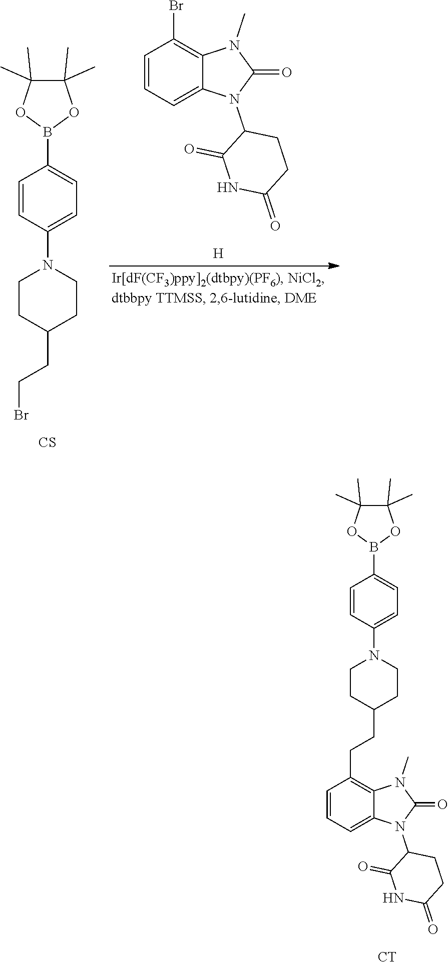

A mixture of 4-(2-bromoethyl)-1-(4-(4,4,5,5-tetramethyl-1,3,2-dioxaborolan-2-yl)phenyl)piperidine (471 mg, 1.40 mmol, Intermediate CS), 3-(4-bromo-3-methyl-2-oxo-2,3-dihydro-1H-benzo[d]imidazol-1-yl)piperidine-2,6-dione (550 mg, 1.40 mmol, Intermediate H), Ir[dF(CF₃)ppy]₂(dtbpy)(PF₆) (157 mg, 40 μmol), NiCl₂·dtbbpy (55.5 mg, 140 μmol) and 2,6-dimethylpyridine (449 mg, 4.19 mmol, 487 μL) in DME (5 mL) was degassed and purged with N₂ three times. Then the mixture was stirred at 25° C. for 12 hrs under N₂ atmosphere. On completion, the mixture was concentrated under reduced pressure. The residue was purified by prep-HPLC (0.8 g/L ammonium bicarbonate) to give the title compound (300 mg, 29% yield) as a white solid. LC-MS (ESI⁺) m/z 573.2 (M+H)⁺.

### (R)-6-(1-acetylpiperidin-3-yl)-7-fluoro-N,N-dimethyl-4-(4-(piperazin-1-yl)-2-(trifluoromethoxy)phenyl)-1H-indole-2-carboxamide (Intermediate CU)

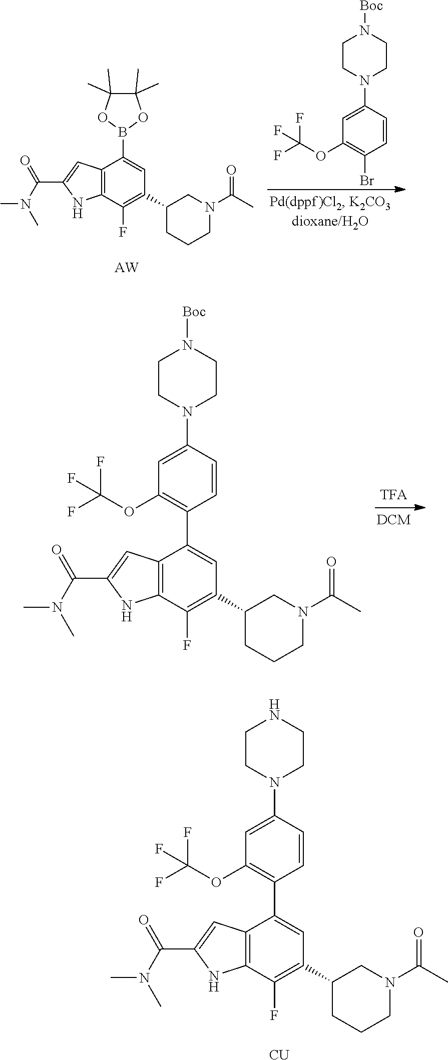

### Step 1—Tert-butyl (R)-4-(4-(6-(1-acetylpiperidin-3-yl)-2-(dimethylcarbamoyl)-7-fluoro-1H-indol-4-yl)-3-(trifluoromethoxy)phenyl)piperazine-1-carboxylate

A mixture of 6-[(3R)-1-acetyl-3-piperidyl]-7-fluoro-N,N-dimethyl-4-(4,4,5,5-tetramethyl -1,3,2-dioxaborolan-2-yl)-1H-indole-2-carboxamide (90.0 mg, 196 μmol, Intermediate AW), tert-butyl 4-[4-bromo-3-(trifluoromethoxy)phenyl]piperazine-1-carboxylate (83.6 mg, 196 mol, synthesized via Step 1 of Intermediate Y), XPhos Pd G3 (17.0 mg, 19.6 μmol), and K₃PO₄ (125 mg, 590 μmol) in dioxane (2 mL) and H₂O (0.5 mL) was degassed and purged with N₂ three times. Then the mixture was stirred at 80° C. for 2 h under N₂ atmosphere. On completion, the reaction mixture was diluted with H₂O (20 mL) and extracted with EA (20 mL×3). The combined organic layers were dried over Na₂SO₄, filtered and concentrated under reduced pressure to give a residue. The residue was purified by flash silica gel chromatography (ISCO®; 4 g SepaFlash® Silica Flash Column, Eluent of 50-100% Ethyl acetate/Petroleum ether gradient @20 mL/min) to afford the title compound (140 mg, 97% yield) as a colorless solid. LC-MS (ESI⁺) m/z 676.1 (M+H)⁺. ¹H NMR (400 MHz, CDCl₃) δ ppm 9.38 (dd, J=1.2, 5.6 Hz, 1H), 7.55-7.44 (m, 1H), 7.22-7.07 (m, 2H), 6.97-6.87 (m, 1H), 6.70-6.51 (m, 1H), 4.86-4.66 (m, 1H), 3.99-3.84 (m, 1H), 3.78 (s, 3H), 3.70-3.63 (m, 1H), 3.33 (s, 6H), 3.25-3.08 (m, 6H), 2.83-2.54 (m, 1H), 2.15 (s, 3H), 2.10 (d, J=10.0 Hz, 1H), 1.93-1.80 (m, 3H), 1.51 (s, 9H).

### Step 2—(R)-6-(1-acetylpiperidin-3-yl)-7-fluoro-N,N-dimethyl-4-(4-(piperazin-1-yl)-2-(trifluoromethoxy)phenyl)-1H-indole-2-carboxamide

To a solution of tert-butyl 4-[4-[6-[(3R)-1-acetyl-3-piperidyl]-2-(dimethylcarbamoyl)-7-fluoro-1H-indol-4-yl]-3-(trifluoromethoxy)phenyl]piperazine-1-carboxylate (140 mg, 207 μmol) in DCM (1.6 mL) was added TFA (430 L, 5.80 mmol). Then the mixture was stirred at 25° C. for 0.5 h. On completion, the reaction mixture was concentrated under reduced pressure to afford the title compound (284 mg, TFA) as a yellow oil. LC-MS (ESI⁺) m/z 576.1 (M+H)⁺.

### 6-(1-(3-(1H-1,2,3-triazol-1-yl)propanoyl)-1,2,5,6-tetrahydropyridin-3-yl)-7-fluoro-N,N- dimethyl-4-(4-(piperazin-1-yl)-2-(trifluoromethoxy)phenyl)-1H-indole-2-carboxamide (Intermediate CV)

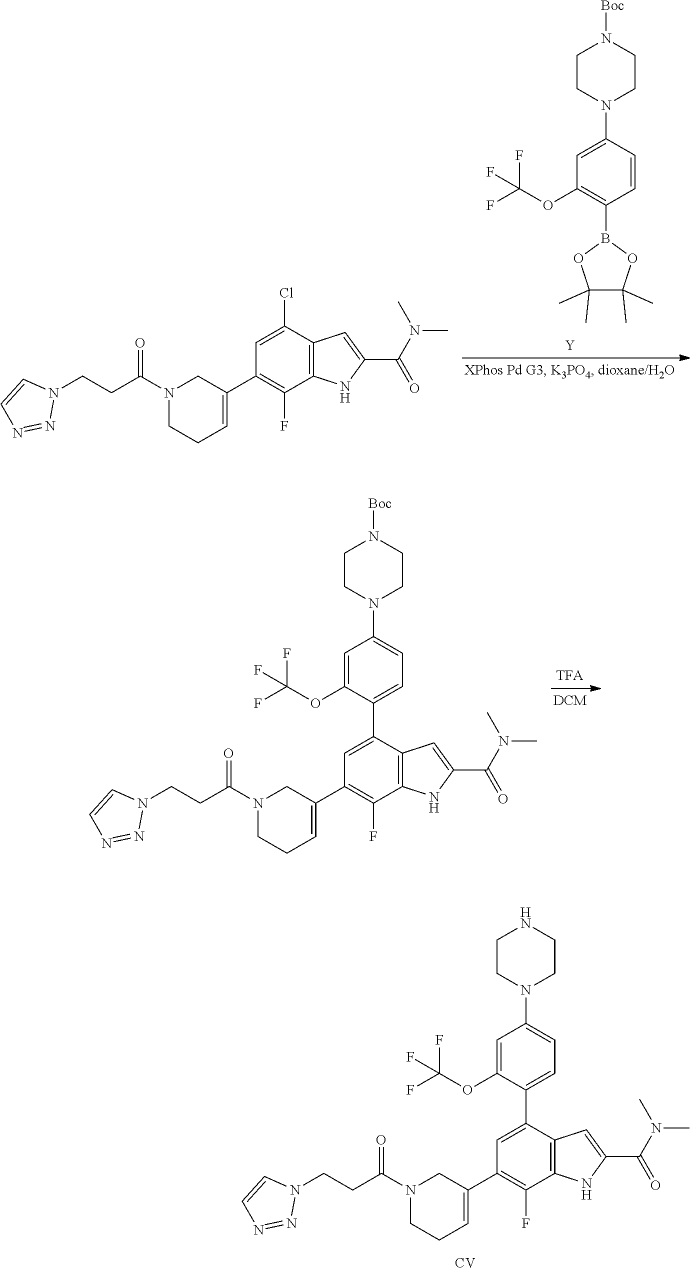

### Step 1—Tert-butyl 4-(4-(6-(1-(3-(1H-1,2,3-triazol-1-yl)propanoyl)-1,2,5,6-tetrahydropyridin-3-yl)-2-(dimethylcarbamoyl)-7-fluoro-1H-indol-4-yl)-3-(trifluoromethoxy)phenyl)piperazine-1-carboxylate

A mixture of 4-chloro-7-fluoro-N,N-dimethyl-6-[1-[3-(triazol-1-yl)propanoyl]-3,6-dihydro-2H-pyridin -5-yl]-1H-indole-2-carboxamide (170 mg, 382 μmol, synthesized via Step 1 of Intermediate AK), tert-butyl 4-[4-(4,4,5,5-tetramethyl-1,3,2-dioxaborolan-2-yl)-3-(trifluoromethoxy)phenyl]piperazine-1-carboxylate (270 mg, 573 μmol, Intermediate Y), XPhos Pd G3 (32.3 mg, 38.2 mol), and K₃PO₄ (243 mg, 1.15 mmol) in dioxane (4.0 mL) and H₂O (1.0 mL) was degassed and purged with N₂ three times, and then the mixture was stirred at 80° C. for 2 h under N₂ atmosphere. On completion, the reaction mixture was diluted with H₂O (20 mL) and extracted with EA (20 mL×3). The combined organic layers were dried over Na₂SO₄, filtered and concentrated under reduced pressure to give a residue. The residue was purified by flash silica gel chromatography (ISCO®; 4 g SepaFlash® Silica Flash Column, Eluent of 50˜100% Ethyl acetate/Petroleum ether gradient @20 mL/min) to give the title compound (270 mg, 89% yield) as a colorless solid. LC-MS (ESI⁺) m/z 755.1 (M+H). ¹H NMR (400 MHz, CDCl₃) δ ppm 9.48-9.38 (m, 1H), 7.78 (d, J=6.0 Hz, 1H), 7.68 (d, J=1.2 Hz, 1H), 7.43 (dd, J=6.0, 8.4 Hz, 1H), 7.12-7.00 (m, 2H), 6.93 (dd, J=6.0, 9.8 Hz, 1H), 6.71-6.58 (m, 1H), 6.15 (d, J=16.0 Hz, 1H), 4.86-4.76 (m, 2H), 4.47 (s, 1H), 4.29 (d, J=1.2 Hz, 1H), 3.78 (t, J=6.0 Hz, 1H), 3.75-3.64 (m, 4H), 3.59 (t, J=6.0 Hz, 1H), 3.40-3.15 (m, 9H), 3.09-3.02 (m, 2H), 2.37 (br s, 2H), 1.51 (s, 9H).

### Step 2—6-(1-(3-(1H-1,2,3-triazol-1-yl)propanoyl)-1,2,5,6-tetrahydropyridin-3-yl)-7-fluoro-N,N- dimethyl-4-(4-(piperazin-1-yl)-2-(trifluoromethoxy)phenyl)-1H-indole-2-carboxamide

To a solution of tert-butyl 4-[4-[2-(dimethylcarbamoyl)-7-fluoro-6-[1-[3-(triazol-1-yl)propanoyl]-3,6-dihydro-2H-pyridin-5-yl]-1H-indol-4-yl]-3-(trifluoromethoxy)phenyl]piperazine-1-carboxylate (250 mg, 331 mol) in DCM (2.8 mL) was added TFA (1.06 g, 9.27 mmol, 688 μL). The mixture was then stirred at 25° C. for 1 hr. On completion, the reaction mixture was concentrated under reduced pressure to afford the title compound (556 mg, TFA) as yellow oil. LC-MS (ESI⁺) m/z 655.2 (M+H) ⁺.

### 2-(1-Acetyl-1,2,5,6-tetrahydropyridin-3-yl)-7-chloro-N,N-dimethyl-1H-indole-5-carboxamide (Intermediate CW)

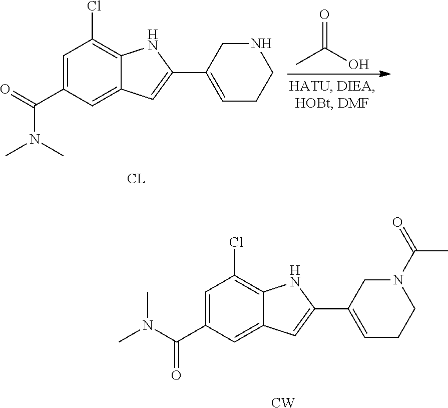

To a solution of AcOH (29 mg, 493 μmol, 28 L, CAS #75-36-5) in DMF (1 mL) was added HATU (162 mg, 427 μmol), HOBt (66 mg, 493 mol), DIEA (212 mg, 1.65 mmol, 286 μL) and 7-chloro-N,N-dimethyl-2-(1,2,5,6-tetrahydropyridin-3-yl)-1H-indole-5-carboxamide (100 mg, 329 μmol, Intermediate CL). The mixture was then stirred at 25° C. for 2 hrs. On completion, the mixture was diluted with water (10 mL) and extracted with ethyl acetate (10 mL×2). The combined organic layer was dried over sodium sulfate, filtered and concentrated under reduced pressure to give the title compound (100 mg) as a black solid. LC-MS (ESI⁺) m/z 346.0 (M+H)⁺.

### 2-(1-Acetyl-1,2,5,6-tetrahydropyridin-3-yl)-7-(2-methoxy-4-(piperazin-1-yl)phenyl)-N,N-dimethyl-1H-indole-5-carboxamide (Intermediate CX)

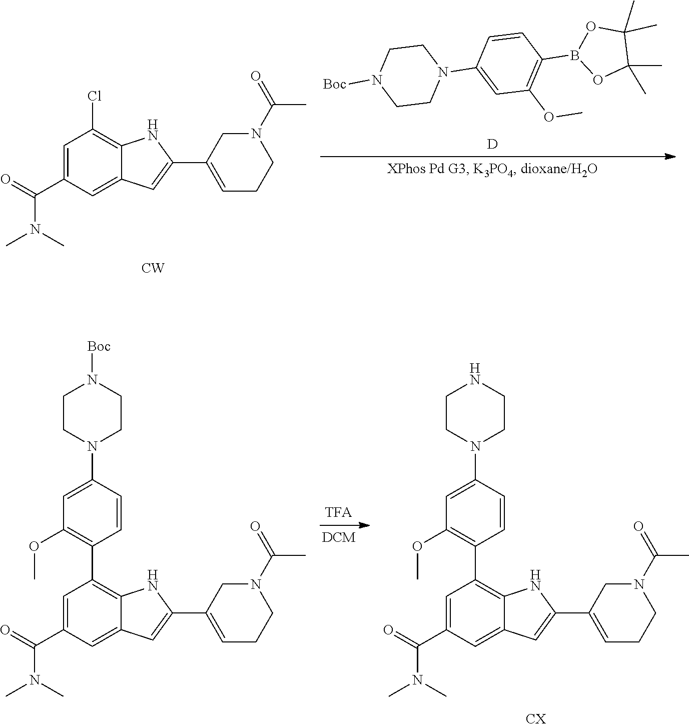

### Step 1—Tert-butyl 4-(4-(2-(I-acetyl-1,2,5,6-tetrahydropyridin-3-yl)-5-(dimethylcarbamoyl)-1H-indol-7-yl)-3-methoxyphenyl)piperazine-1-carboxylate

A mixture of 2-(1-acetyl-1,2,5,6-tetrahydropyridin-3-yl)-7-chloro-N,N-dimethyl-1H-indole-5-carboxamide (100 mg, 578 μmol, Intermediate CW), tert-butyl 4-(3-methoxy-4-(4,4,5,5-tetramethyl-1,3,2-dioxaborolan-2-yl)phenyl)piperazine-1-carboxylate (121 mg, 289 mol, Intermediate D), K₃PO₄ (184 mg, 867 mol), and XPhos Pd G3 (24.5 mg, 28.9 μmol) in dioxane (5 mL) and H₂O (1.5 mL) was degassed and purged with N₂ three times. Then the mixture was stirred at 80° C. for 1 hr under N₂ atmosphere. On completion, the reaction mixture was diluted by water (10 mL) and extracted by ethyl acetate (3×20 mL). The combined organic layers were washed with brine (10 mL) and dried over anhydrous sodium sulfate, filtered and the filtrate was concentrated in vacuo to give the crude residue. The crude residue was purified by column chromatography (SiO₂, Petroleum ether/Ethyl acetate=10/0 to 1/1) to give the title compound (110 mg, 56% yield) as a yellow solid. LC-MS (ESI⁺) in z 602.2 (M+H)f.

### Step 2—2-(1-Acetyl-1,2,5,6-tetrahydropyridin-3-yl)-7-(2-methoxy-4-(piperazin-1-yl)phenyl)-N,N-dimethyl-1H-indole-5-carboxamide

To a solution of tert-butyl 4-(4-(2-(1-acetyl-1,2,5,6-tetrahydropyridin-3-yl)-5-(dimethylcarbamoyl)-1H-indol-7-yl)-3-methoxyphenyl)piperazine-1-carboxylate (110 mg, 183 μmol) in DCM (3 mL) was added TFA (1.39 g, 13.9 mmol, 1 mL). The mixture was then stirred at 25° C. for 2 hrs. On completion, the reaction mixture was concentrated in vacuo to give the title compound (300 mg, TFA) as a gray solid. LC-MS (ESI⁺) m/z 502.2 (M+H)⁺.

### 2-(1-(3-(1H-pyrazol-1-yl)propanoyl)-1,2,5,6-tetrahydropyridin-3-yl)-4-chloro-N,N-dimethylbenzo[d]thiazole-6-carboxamide (Intermediate CY)

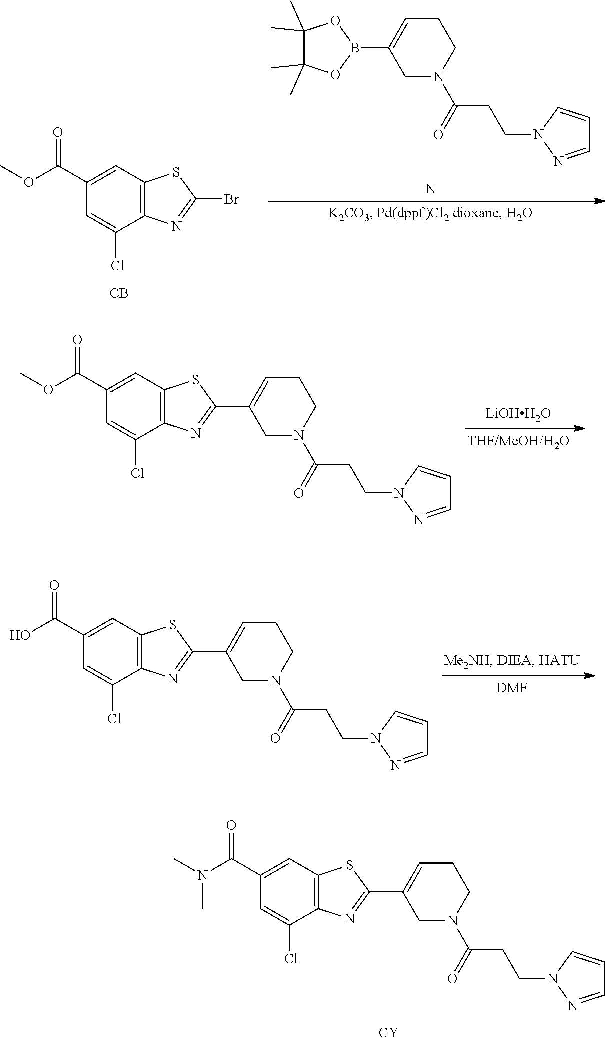

### Step 1—Methyl 2-(1-(3-(1H-pyrazol-1-yl)propanoyl)-1,2,5,6-tetrahydropyridin-3-yl)-4-chlorobenzo[d]thiazole-6-carboxylate

A mixture of methyl 2-bromo-4-chlorobenzo[d]thiazole-6-carboxylate (3 g, 7.83 mmol, Intermediate CB), 3-(1H-pyrazol-1-yl)-1-(5-(4,4,5,5-tetramethyl-1,3,2-dioxaborolan-2-yl)-3,6-dihydropyridin-1(2H)-yl)propan-1-one (2.59 g, 7.83 mmol. Intermediate N), Pd(dppf)Cl₂ (572 mg, 782 mol), and K₂CO₃ (3.25 g, 23.4 mmol) in dioxane (5 mL) and H₂O (0.5 mL) was degassed and purged with N₂ three times. Then the mixture was stirred at 80° C. for 2 hrs under N₂ atmosphere. On completion, the reaction mixture was diluted with water (20 mL) and extracted with dichloromethane (2×20 mL). The combined organic layers were washed with brine (40 mL) and dried over anhydrous sodium sulfate, filtered and the filtrate was concentrated in vacuo to give the crude residue. The residue was purified by column chromatography (SiO₂, Petroleum ether:Ethyl acetate=1/1 to 0/1) to give the title compound (1.8 g, 38% yield) as a yellow solid. LC-MS (ESI⁺) m/z 431.0 (M+H)⁺.

### Step 2—2-(1-(3-(1H-pyrazol-1-yl)propanoyl)-1,2,5,6-tetrahydropyridin-3-yl)-4-chlorobenzo[d]thiazole-6-carboxylic acid

To a solution of methyl 2-(1-(3-(1H-pyrazol-1-yl)propanoyl)-1,2,5,6-tetrahydropyridin-3-yl)-4-chlorobenzo[d]thiazole-6-carboxylate (1.8 g, 4.18 mmol) in THF (8 mL) H₂O (2 mL) and MeOH (2 mL) was added LiOH·H₂O (350 mg, 8.35 mmol). The mixture was then stirred at 25° C. for 2 hrs. On completion, the reaction mixture was added HCl (1N) until the pH-5, then diluted by water (10 mL) and extracted by ethyl acetate (2×20 mL). The combined organic layers were dried over anhydrous sodium sulfate, filtered and the filtrate was concentrated in vacuo to give the title compound (1.8 g) as yellow solid. LC-MS (ESI⁺) m/z 417.0 (M+H)⁺.

### Step 3—2-(1-(3-(1H-pyrazol-1-yl)propanoyl)-1,2,5,6-tetrahydropyridin-3-yl)-4-chloro-N,N-dimethylbenzo[d]thiazole-6-carboxamide

To a solution of 2-(1-(3-(1H-pyrazol-1-yl)propanoyl)-1,2,5,6-tetrahydropyridin-3-yl)-4-chlorobenzo[d]thiazole-6-carboxylic acid (1.5 g, 3.60 mmol) in DMF (10 mL) was added HATU (1.64 g, 4.32 mmol). DIEA (2.33 g, 17.9 mmol, 3.13 mL) and N-methylmethanamine hydrochloride (293 mg, 3.60 mmol). The mixture was then stirred at 25° C. for 1 hr. On completion, the reaction mixture was diluted with water (10 mL) and extracted with dichloromethane (2×20 mL). The combined organic layers were washed with brine (20 mL) and dried over anhydrous sodium sulfate, filtered and the filtrate was concentrated in vacuo to give the crude residue. The residue was purified by reverse phase HPLC (0.1% FA condition) to give the title compound (1 g, 63% yield) as a white solid. LC-MS (ESI′) m/z 443.9 (M+H)⁺.

### 2-(1-(3-(1H-pyrazol-1-yl)propanoyl)-1,2,5,6-tetrahydropyridin-3-yl)-N,N-dimethyl-4-(3-(piperazin-1-yl)phenyl)benzo[d]thiazole-6-carboxamide (Intermediate CZ)

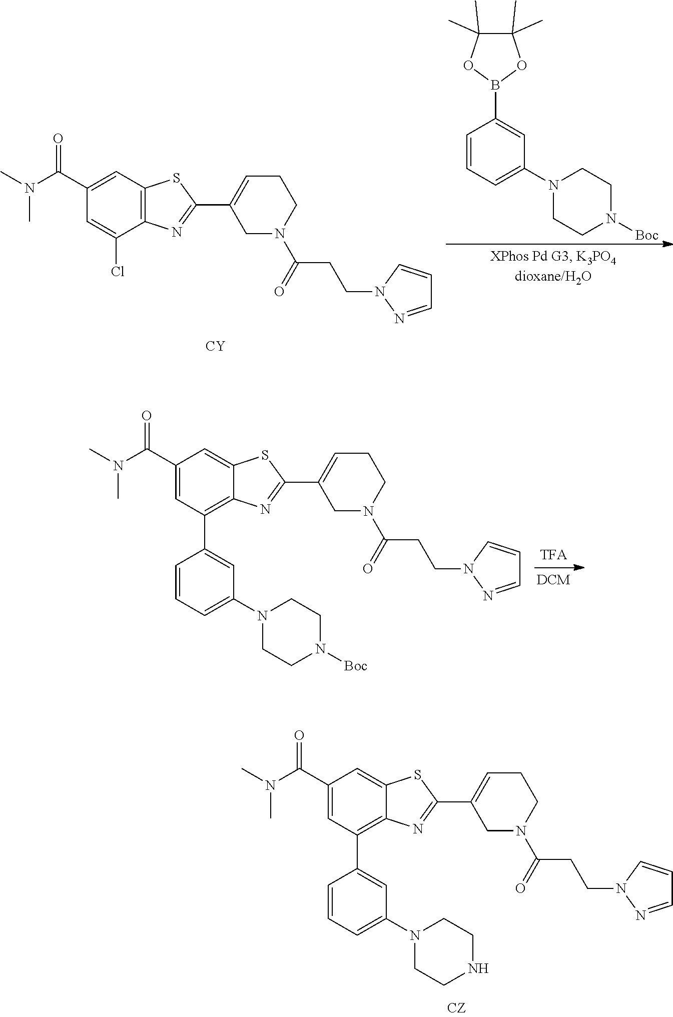 [SMILES: CN(C)C(=O)c1cc(Cl)c2nc(C3=CCCN(C(=O)CCn4cccn4)C3)sc2c1 ; CC(C)(C)OC(=O)N1CCN(c2cccc(B3OC(C)(C)C(C)(C)O3)c2)CC1 ; "CY" ; CN(C)C(=O)c1cc(-c2cccc(N3CCN(C(=O)OC(C)(C)C)CC3)c2)c2nc(C3=CCCN(C(=O)CCn4cccn4)C3)sc2c1 ; CN(C)C(=O)c1cc(-c2cccc(N3CCNCC3)c2)c2nc(C3=CCCN(C(=O)CCn4cccn4)C3)sc2c1]

### Step 1—Tert-butyl 4-(3-(2-(1-(3-(1H-pyrazol-1-yl)propanoyl)-1,2,5,6-tetrahydropyridin-3-yl)-6-(dimethylcarbamoyl)benzo[d]thiazol-4-yl)phenyl)piperazine-1-carboxylate

A mixture of 2-(1-(3-(1H-pyrazol-1-yl)propanoyl)-1,2,5,6-tetrahydropyridin-3-yl)-4-chloro-N,N-dimethylbenzo[d]thiazole-6-carboxamide (100 mg, 225 μmol, Intermediate CY), tert-butyl 4-(3-(4,4,5,5-tetramethyl-1,3,2-dioxaborolan-2-yl)phenyl)piperazine-1-carboxylate (87.4 mg, 225 μmol, CAS #540752-87-2), XPhos Pd G3 (19.0 mg, 22.5 μmol), and K₃PO₄ (143 mg, 675 mol) in dioxane (10 mL) and H₂O (1 mL) was degassed and purged with N₂ three times. Then the mixture was stirred at 80° C. for 5 hrs under N₂ atmosphere. The reaction mixture was diluted with water (10 mL) and extracted with dichloromethane (2×20 mL). The combined organic layers were washed with brine (20 mL) and dried over anhydrous sodium sulfate, filtered and the filtrate was concentrated in vacuo to give the crude residue. The residue was purified by reverse phase HPLC (0.1% FA condition) to give the title compound (100 mg, 66% yield) as a white solid. LC-MS (ESI⁺) m/z 670.3 (M+H)⁺.

### Step 2—2-(1-(3-(1H-pyrazol-1-yl)propanoyl)-1,2,5,6-tetrahydropyridin-3-yl)-N,N-dimethyl-4-(3-(piperazin-1-yl)phenyl)benzo[d]thiazole-6-carboxamide

To a solution of tert-butyl 4-(3-(2-(1-(3-(1H-pyrazol-1-yl)propanoyl)-1,2,5,6-tetrahydropyridin-3-yl)-6-(dimethylcarbamoyl)benzo[d]thiazol-4-yl)phenyl)piperazine-1-carboxylate (100 mg, 149 μmol) in DCM (10 mL) was added TFA (1.54 g, 13.4 mmol, 1 mL). The mixture was then stirred at 25° C. for 2 hrs. On completion, the mixture was concentrated under reduced pressure to give the title compound (80 mg, 94% yield) as brown solid. LC-MS (ESI⁺) m/z 570.3 (M+H)⁺.

### Tert-butyl (R)-3-(4-chloro-2-(dimethylcarbamoyl)-7-fluoro-1H-indol-6-yl)piperidine-1-carboxylate (Intermediate DA) & tert-butyl (S)-3-(4-chloro-2-(dimethylcarbamoyl)-7-fluoro-1H-indol-6-yl)piperidine-1-carboxylate (Intermediate DB)

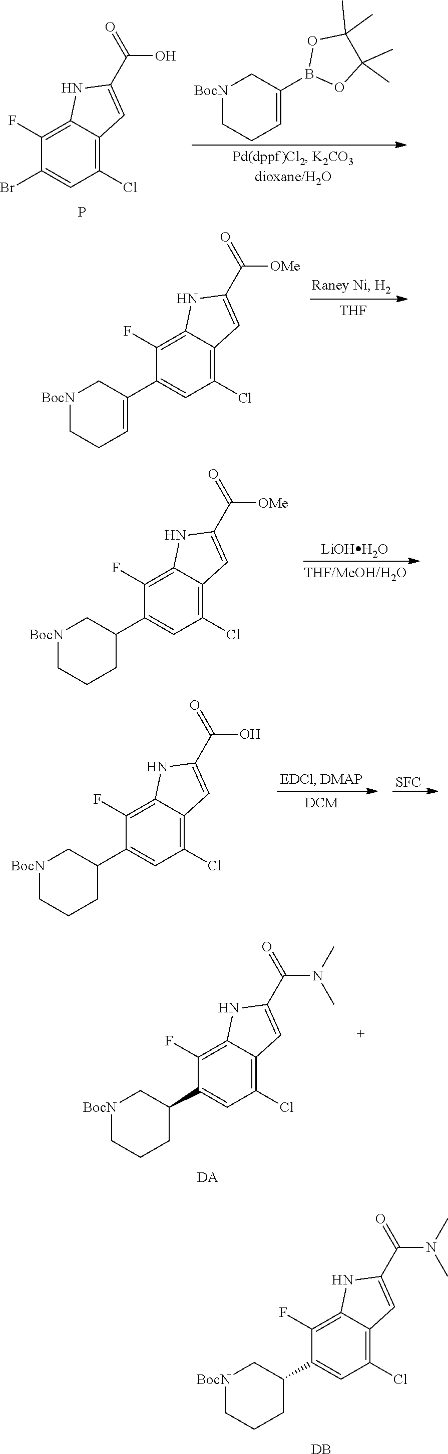

### Step 1—Methyl 6-(1-(tert-butoxycarbonyl)-1,2,5,6-tetrahydropyridin-3-yl)-4-chloro-7-fluoro-1H-indole-2-carboxylate

To a solution of methyl 6-bromo-4-chloro-7-fluoro-1H-indole-2-carboxylate (1 g, 3.26 mmol. Intermediate P) and tert-butyl 5-(4,4,5,5-tetramethyl-1,3,2-dioxaborolan-2-yl)-3,6-dihydropyridine-1(2H)-carboxylate (1.01 g, 3.26 mmol, CAS #885693-20-9) in dioxane (10 mL) and H₂O (3 mL) was added Pd(dppf)Cl₂ (238 mg, 326 μmol) and K₂CO₃ (1.35 g, 9.79 mmol). The mixture was then stirred at 60° C. for 1 hr. On completion, the reaction mixture was diluted with water (100 mL) and extracted with DCM (100 mL×3). The combined organic layers were washed with brine (100 mL×2), dried over Na₂SO₄, filtered and concentrated under reduced pressure to give a residue. The crude product was triturated with ACN (100 mL) at 20° C. for 30 min, then filtered to give the title compound (1.2 g) as a yellow solid LC-MS (ESI⁺) m/z 353.0 (M+H-56)⁺.

### Step 2—Methyl 6-(1-(tert-butoxycarbonyl)piperidin-3-yl)-4-chloro-7-fluoro-1H-indole-2-carboxylate

To a solution of methyl 6-(1-(tert-butoxycarbonyl)-1,2,5,6-tetrahydropyridin-3-yl)-4-chloro-7-fluoro-1H-indole-2-carboxylate (560 mg, 1.37 mmol) in THF (15 mL) was added Raney-Ni (469 mg, 5.48 mmol) under N₂ atmosphere. The suspension was degassed and purged with H₂ three times. Then the mixture was stirred under H₂ (15 PSI) at 25° C. for 10 hrs. On completion, the reaction was filtered through kieselguhr very carefully, and the filtrate was concentrated in vacuo to give the title compound (490 mg) as a light yellow solid. LC-MS (ESI) m/z 355.0 (M+H-56)⁺.

### Step 3—6-(1-(Tert-butoxycarbonyl)piperidin-3-yl)-4-chloro-7-fluoro-1H-indole-2-carboxylic acid

To a solution of methyl 6-(1-(tert-butoxycarbonyl)piperidin-3-yl)-4-chloro-7-fluoro-1H-indole-2-carboxylate (490 mg, 834 μmol) in THF (4 mL), H₂O (1 mL) and MeOH (1 mL) was added LiOH·H₂O (280 mg, 6.68 mmol). The mixture was then stirred at 25° C. for 12 hrs. On completion, HCl (1N) was added to the reaction mixture until the pH=7, then diluted with water (50 mL) and extracted with ethyl acetate (3×50 mL). The combined organic layers were dried over anhydrous sodium sulfate, filtered and the filtrate was concentrated in vacuo to give the title compound (435 mg) as a yellow solid. LC-MS (ESI⁺) m/z 341.0 (M+H-56)f.

### Step 4—Tert-butyl (R)-3-(4-chloro-2-(dimethylcarbamoyl)-7-fluoro-TH-indol-6-yl)piperidine-1-carboxylate & tert-butyl (S)-3-(4-chloro-2-(dimethylcarbamoyl)-7-fluoro-1H-indol-6-yl)piperidine-1-carboxylate

To a solution of 6-(I-(tert-butoxycarbonyl)piperidin-3-yl)-4-chloro-7-fluoro-1H-indole-2-carboxylic acid (400 mg, 1.01 mmol) in DCM (8 mL) was added EDCI (231 mg, 1.21 mmol) and DMAP (492 mg, 4.03 mmol) and the mixture was stirred at 20° C. for 1 hr. Then N-methylmethanamine hydrochloride (123 mg, 1.51 mmol) was added and the mixture was stirred at 20° C. for 11 hrs. On completion, the reaction mixture was concentrated under reduced pressure to give a residue and the crude product was triturated with water (20 mL) at 20° C. for 15 min. The residue was purified by prep-HPLC (neutral condition) and then purified by SFC (column: DAICEL CHIRALPAK AD(250 mm*30 mm, 10 um): mobile phase: [CO₂-i-PrOH]; B %: 40%, isocratic elution mode column to give the tert-butyl (R)-3-(4-chloro-2-(dimethylcarbamoyl)-7-fluoro-1H-indol-6-yl)piperidine-1-carboxylate (75 mg, 17% yield) as a white solid and tert-butyl (S)-3-(4-chloro-2-(dimethylcarbamoyl)-7-fluoro-TH-indol-6-yl)piperidine-1-carboxylate (90 mg, 21% yield) as a white solid. LC-MS (ESI) m/z 368.3 (M+H-56)⁻ for both isomers. The absolute stereochemistry of the enantiomers was assigned arbitrarily.

### (R)-6-(1-(tert-butoxycarbonyl)piperidin-3-yl)-4-chloro-7-fluoro-1H-indole-2-carboxylic acid (Intermediate DC)

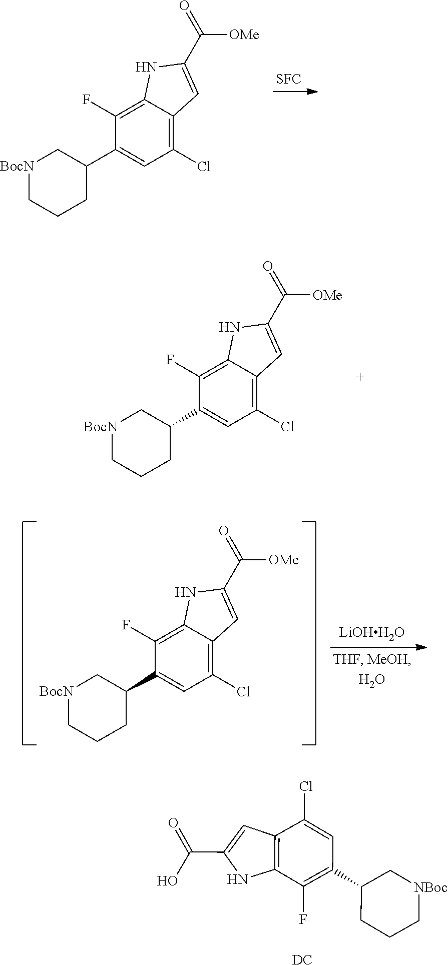

### Step 1—Methyl (R)-6-(1-(tert-butoxycarbonyl)piperidin-3-yl)-4-chloro-7-fluoro-1H-indole-2-carboxylate

Methyl 6-(1-(tert-butoxycarbonyl)piperidin-3-yl)-4-chloro-7-fluoro-1H-indole-2-carboxylate (50.0 g, 122 mmol, synthesized via Steps 1-2 of Intermediate DA) was purified by Prep-HPLC (column: DAICEL CHIRALPAK IG (250 mm*30 mm, 10 um); mobile phase: [CO₂—MeOH (0.1% NH₃H₂O)]; B %: 30%, isocratic elution mode) to give methyl (R)-6-(1-(tert-butoxycarbonyl)piperidin-3-yl)-4-chloro-7-fluoro-1H-indole-2-carboxylate (18.5 g, 74% yield) as a white solid (LC-MS (ESI) m/z 355.1; ¹H NMR 400 MHz, DMSO-d₆ δ 12.8 (s, 1H), 7.17 (d, J=5.2 Hz, 1H), 7.12 (d, J=2.8 Hz, 1H), 3.96 (d, J=12.4 Hz, 2H), 3.89 (s, 3H), 3.04˜2.99 (m, 3H), 1.84˜1.73 (m, 3H), 1.49˜1.46 (m, 1H), 1.41 (s, 9H)) and methyl (S)-6-(1-(tert-butoxycarbonyl)piperidin-3-yl)-4-chloro-7-fluoro-1H-indole-2-carboxylate (20.0 g, 80.0% yield) as a white solid (LC-MS (ESI⁺) m/z 355.1; ¹H NMR 400 MHz, DMSO-d₆ δ 12.9-12.7 (m, 1H), 7.17 (d, J=4.8 Hz, 1H), 7.12 (d, J=2.8 Hz, 1H), 3.96 (d, J=12.0 Hz, 2H), 3.89 (s, 3H), 3.04˜2.84 (m, 3H), 1.88˜1.70 (m, 3H), 1.49˜1.42 (m, 1H), 1.41 (s, 9H)). Absolute stereochemistry of the enantiomers were assigned arbitrarily.

### Step 2—(R)-6-(1-(tert-butoxycarbonyl)piperidin-3-yl)-4-chloro-7-fluoro-1H-indole-2-carboxylic acid

To a solution of methyl (R)-6-(1-(tert-butoxycarbonyl)piperidin-3-yl)-4-chloro-7-fluoro-1H-indole-2-carboxylate (2 g, 4.87 mmol) in THF (16 mL), MeOH (4 mL) and H₂O (4 mL) was added LiOH·H₂O (612 mg, 14.6 mmol). The mixture was stirred at 25° C. for 12 hrs. On completion, the reaction mixture was diluted with water (60 mL) and extracted with ethyl acetate (3×60 mL). The combined organic layers were washed with brine (120 mL) and dried over anhydrous sodium sulfate, filtered and the filtrate was concentrated in vacuo to give title compound (2.1 g) as a blue solid. LC-MS (ESI⁺) m/z 341.0 (M-55) ⁺.

### (R)-azetidin-1-yl(4-chloro-7-fluoro-6-(piperidin-3-yl)-1H-indol-2-yl)methanone (Intermediate DD)

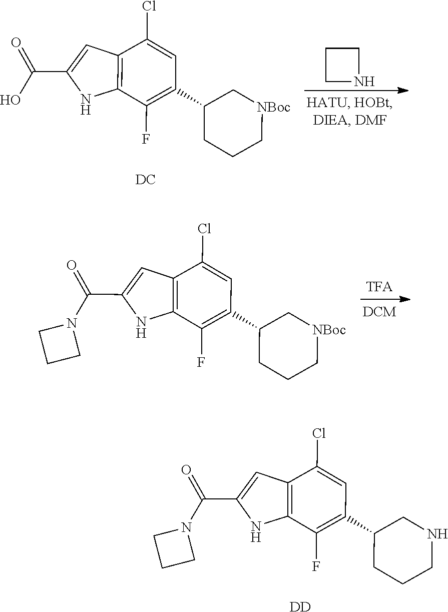

### Step 1—Tert-butyl (R)-3-(2-(azetidine-1-carbonyl)-4-chloro-7-fluoro-1H-indol-6-yl)piperidine-1-carboxylate

To a solution of 6-[(3R)-1-tert-butoxycarbonyl-3-piperidyl]-4-chloro-7-fluoro-1H-indole-2-carboxylic acid (1 g, 3 mmol, Intermediate DC) in DMF (10 mL) was added HATU (1.05 g, 2.77 mmol), HOBt (681 mg, 5.04 mmol), DIEA (1.63 g, 12.6 mmol) and azetidine (943.0 mg, 10.1 mmol, CAS #503-29-7). Then the mixture was stirred at 25° C. for 10 mins. On completion, water (80 ml) was added then the mixture was filtered. The filter cake was concentrated in vacuo to give the title compound (1 g) as an off-white solid. LC-MS (ESI⁺) m/z 380.1 (M-55)⁺.

### Step 2—(R)-azetidin-1-yl(4-chloro-7-fluoro-6-(piperidin-3-yl)-1H-indol-2-yl)methanone

To a solution of tert-butyl (R)-3-(2-(azetidine-1-carbonyl)-4-chloro-7-fluoro-1H-indol-6-yl)piperidine-1-carboxylate (900 mg, 2.06 mmol) in DCM (15 mL) was added TFA (7.68 g, 67.3 mmol). The mixture was stirred at 25° C. for 30 mins. On completion, the reaction mixture was quenched with NaHCO₃ until the mixture was neutral, then extracted with dichloromethane (3×180 mL). The combined organic layers were washed with brine (120 mL) and dried over anhydrous sodium sulfate, filtered and the filtrate was concentrated in vacuo to give the title compound (1 g, TFA) as a yellow solid. LC-MS (ESI⁺) m/z 336.1 (M+H)⁺.

### (R)-1-(3-(2-(azetidine-1-carbonyl)-7-fluoro-4-(4,4,5,5-tetramethyl-1,3,2-dioxaborolan-2-yl)-1H-indol-6-yl)piperidin-1-yl)-3-(1H-pyrazol-1-yl)propan-1-one (Intermediate DE)

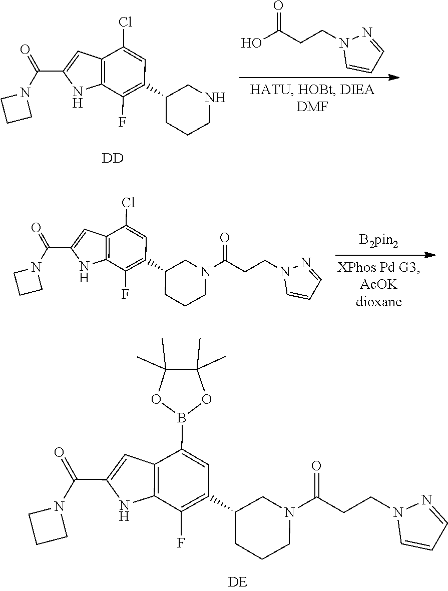 [SMILES: O=C(c1cc2c(Cl)cc([C@H]3CCCNC3)c(F)c2[nH]1)N1CCC1 ; O=C(O)CCn1cccn1 ; "DD" ; O=C(CCn1cccn1)N1CCC[C@H](c2cc(Cl)c3cc(C(=O)N4CCC4)[nH]c3c2F)C1 ; CC1(C)OB(c2cc([C@H]3CCCN(C(=O)CCn4cccn4)C3)c(F)c3[nH]c(C(=O)N4CCC4)cc23)OC1(C)C]

### Step 1—(R)-1-(3-(2-(azetidine-1-carbonyl)-4-chloro-7-fluoro-1H-indol-6-yl)piperidin-1-yl)-3-(1H-pyrazol-1-yl)propan-1-one

To a solution of azetidin-1-yl-[4-chloro-7-fluoro-6-[(3R)-3-piperidyl]-1H-indol-2-yl]methanone (950 mg, 2.11 mmol, TFA, Intermediate DD) in DMF (10 mL) was added HATU (883 mg, 2.32 mmol), HOBt (570 mg, 4.22 mmol), DIEA (1.36 g, 10.6 mmol) and 3-pyrazol-1-ylpropanoic acid (888 mg, 6.34 mmol, CAS #89532-73-0). Then the mixture was stirred at 25° C. for 10 mins. On completion, water (80 mL) was added to the mixture and a solid precipitated. The mixture was filtered and the filter cake was dried in vacuo to give the title compound (700 mg) as a white solid. LC-MS (ESI⁺) m/z 458.1 (M+H)⁺.

### Step 2—(R)-1-(3-(2-(azetidine-1-carbonyl)-7-fluoro-4-(4,4,5,5-tetramethyl-1,3,2-dioxaborolan-2-yl)-1H-indol-6-yl)piperidin-1-yl)-3-(1H-pyrazol-1-yl)propan-1-one

To a solution of (R)-1-(3-(2-(azetidine-1-carbonyl)-4-chloro-7-fluoro-1H-indol-6-yl)piperidin-1-yl)-3-(1H-pyrazol-1-yl)propan-1-one (390 mg, 852 mol) and 4,4,5,5-tetramethyl-2-(4,4,5,5-tetramethyl-1,3,2-dioxaborolan-2-yl)-1,3,2-dioxaborolane (1.08 g, 4.26 mmol, CAS #73183-34-3) in dioxane (10 mL) was added XPhos Pd G3 (144 mg, 170 μmol) and AcOK (251 mg, 2.56 mmol). The mixture was then stirred at 80° C. for 6 hrs. On completion, the reaction mixture was quenched/diluted with water (50 mL) and extracted with ethyl acetate (3×50 mL). The combined organic layers were washed with brine (100 mL) and dried over anhydrous sodium sulfate, filtered and the filtrate was concentrated in vacuo to give the crude residue. The residue was purified by column chromatography (SiO₂, Dichloromethane:Methanol=10/1) to give the title compound (330 mg, 60% yield) as a black solid. LC-MS (ESI⁺) m/z 550.2 (M+H)⁺.

### 7-(4-Chlorophenyl)-2,7-diazaspiro[3.5]nonane (Intermediate DF)

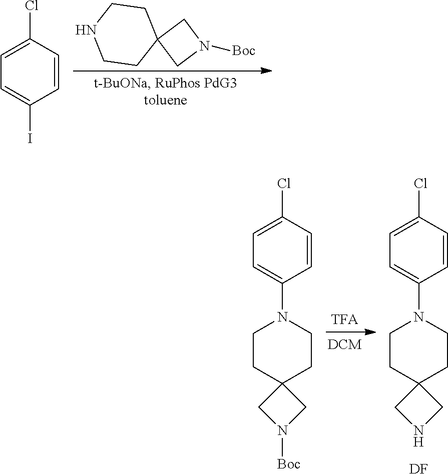

### Step 1—Tert-butyl 7-(4-chlorophenyl)-2,7-diazaspiro[3.5]nonane-2-carboxylate

A mixture of tert-butyl 2,7-diazaspiro[3.5]nonane-2-carboxylate (5.69 g, 25.1 mmol, CAS #236406-55-6), 1-chloro-4-iodo-benzene (5.00 g, 20.9 mmol, CAS #637-87-6), t-BuONa (2.42 g, 25.1 mmol), and RuPhos PdG3 (350 mg, 419 μmol) in toluene (60.0 mL) was degassed and purged with N₂three times. Then the mixture was stirred at 100° C. for 2 h under N₂ atmosphere. On completion, the mixture was concentrated under reduced pressure using a rotary evaporator. The residue was purified by silica gel column chromatography (petroleum ether˜petroleum ether/ethyl acetate=20/1) to give the title compound (6.00 g, 76% yield) as a white solid. LC-MS (ESI⁺) m/z 337.2 (M+H)⁺.

### Step 2—7-(4-Chlorophenyl)-2,7-diazaspiro[3.5]nonane

To a solution of tert-butyl 7-(4-chlorophenyl)-2,7-diazaspiro[3.5]nonane-2-carboxylate (2.00 g, 5.94 mmol) in DCM (10.0 mL) was added TFA (7.67 g, 67.3 mmol, 5.0 mL). The mixture was then stirred at 20° C. for 2 hr. On completion, the mixture was concentrated under reduced pressure using a rotary evaporator. The mixture was diluted with water (15 mL) and extracted with ethyl acetate (15 mL×2). The organic layer was washed with sat. aq. of NaHCO₃, dried over sodium sulfate, filtered and concentrated under reduced pressure to give the title compound (1.42 g) as a yellow oil. solid. LC-MS (ESI⁺) m/z 237.2 (M+H)⁺.

### 3-(4-(7-(4-Chlorophenyl)-2,7-diazaspiro[3.5]nonan-2-yl)-3-methyl-2-oxo-2,3-dihydro-1H-benzo[d]imidazol-1-yl)piperidine-2,6-dione (Intermediate DG)

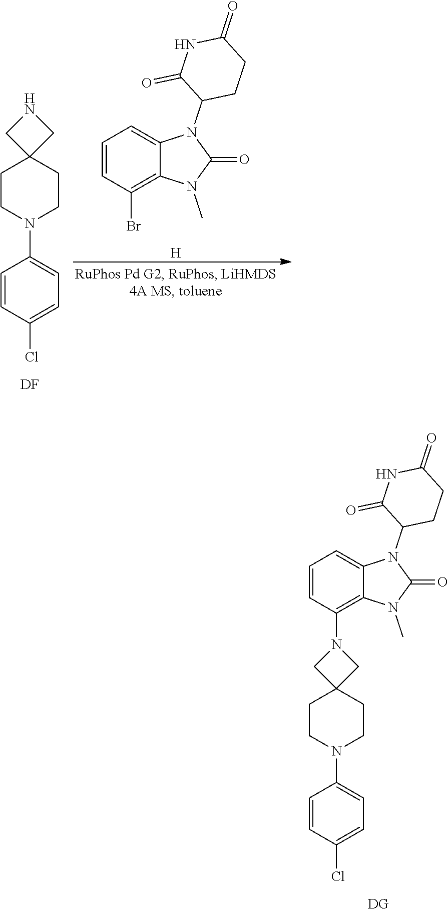

To a solution of 7-(4-chlorophenyl)-2,7-diazaspiro[3.5]nonane (500 mg, 2.11 mmol, Intermediate DF), 3-(4-bromo-3-methyl-2-oxo-2,3-dihydro-1H-benzo[d]imidazol-1-yl)piperidine-2,6-dione (714.2 mg, 2.11 mmol, Intermediate H) and 4A molecular sieves (1.5 g) in toluene (20 mL) was added LiHMDS (1 M, 12.7 mL) at 0° C. After 30 minutes, RuPhos (148 mg, 317 μmol) and [2-(2-aminophenyl)phenyl]-chloro-palladium dicyclohexyl-[2-(2,6-diisopropoxyphenyl)phenyl]phosphane (164 mg, 211 μmol) was added and the mixture was stirred at 100° C. for 2 hrs. On completion, the reaction mixture was concentrated under reduced pressure to remove solvent. The crude residue was purified by reverse phase HPLC (0.8 g/L ammonium bicarbonate) to give the title compound (350 mg, 27% yield) as a white solid. LC-MS (ESI⁺) m/z 494.2 (M+H)⁺.

### 1-(2,6-Dioxopiperidin-3-yl)-3-methyl-2-oxo-2,3-dihydro-1H-benzo[d]imidazole-4-carbaldehyde (Intermediate DH)

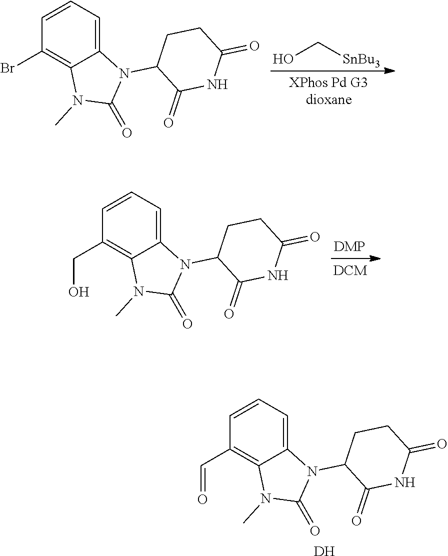

### Step 1—3-(4-(Hydroxymethyl)-3-methyl-2-oxo-2,3-dihydro-1H-benzo[d]imidazol-1-yl)piperidine-2,6-dione

A mixture of 3-(4-bromo-3-methyl-2-oxo-2,3-dihydro-1H-benzo[d]imidazol-1-yl)piperidine-2,6-dione (1 g, 2.96 mmol, Intermediate H),XPhos Pd G3 (250 mg, 295 mol), tributylstannylmethanol (1.42 g, 4.44 mmol, CAS #27490-33-1) in dioxane (10 mL) was degassed and purged with N₂ three times. Then the mixture was stirred at 80° C. for 12 hrs under N₂ atmosphere. On completion, the reaction mixture was quenched with water (20 mL) and extracted with dichloromethane (2×20 mL). The combined organic layers were washed with brine (20 mL) and dried over anhydrous sodium sulfate, filtered and the filtrate was concentrated in vacuo to give the crude residue. The residue was purified by reverse phase HPLC (0.1% FA condition) to give the title compound (550 mg, 64% yield) as a white solid. LC-MS (ESI⁺) m/z 290.2 (M+H)⁺.

### Step 2—1-(2,6-Dioxopiperidin-3-yl)-3-methyl-2-oxo-2,3-dihydro-1H-benzo[d]imidazole-4-carbaldehyde

To a solution of 3-(4-(hydroxymethyl)-3-methyl-2-oxo-2,3-dihydro-1H-benzo[d]imidazol-1-yl)piperidine-2,6-dione (100 mg, 345 μmol) in DCM (1 mL) was added DMP (219 mg, 518 μmol, 160 μL). The mixture was stirred at 25° C. for 1 hr. On completion, the mixture was quenched by Na₂SO₃ (5 mL) and added NaHCO₃ until the pH 6, then extracted with dichloromethane (2×5 mL). The combined organic layers were washed with brine (10 mL) and dried over anhydrous sodium sulfate, filtered and the filtrate was concentrated in vacuo to give the crude residue. The crude residue was purified by reverse phase HPLC (0.1% FA condition) to give the title compound (50 mg, 50% yield) as a white solid. LC-MS (ESI⁺) m/z 288.1 (M+H)⁺.

### 6-(1-(3-(1H-pyrazol-1-yl)propanoyl)-1,2,5,6-tetrahydropyridin-3-yl)-7-fluoro-4-(2-methoxy-4-(piperazin-1-yl)phenyl)-N,N-dimethyl-1H-indole-2-carboxamide (Intermediate DI)

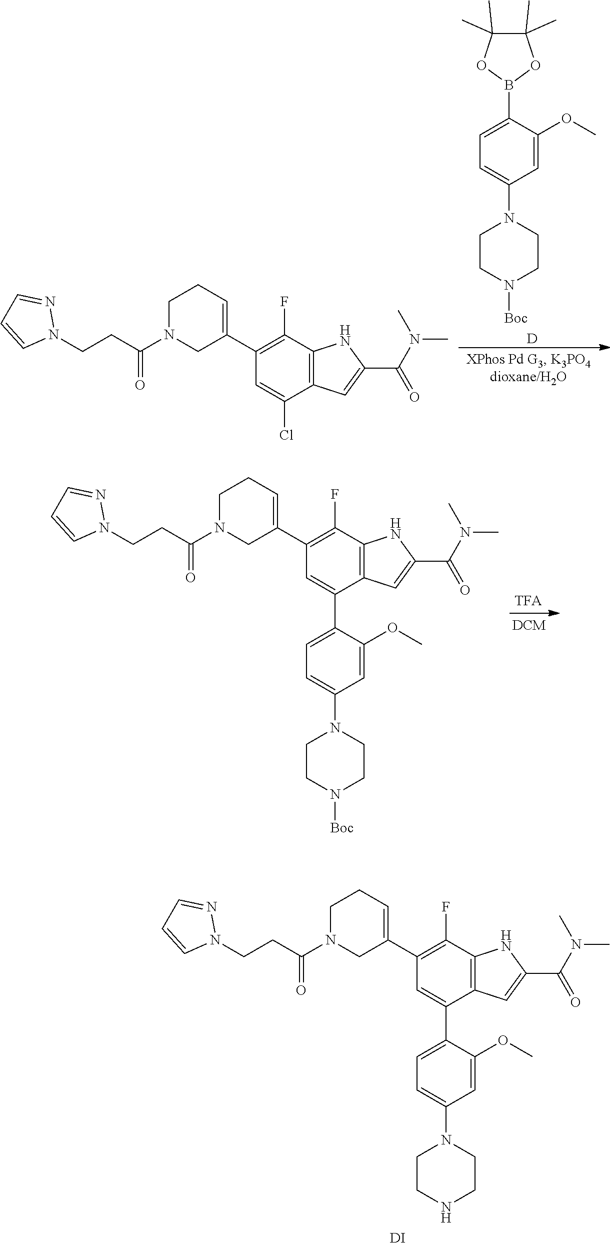

### Step 1—Tert-butyl 4-(4-(6-(1-(3-(1H-pyrazol-1-yl)propanoyl)-1,2,5,6-tetrahydropyridin-3-yl)-2-(dimethylcarbamoyl)-7-fluoro-1H-indol-4-yl)-3-methoxyphenyl)piperazine-1-carboxylate

A mixture of 6-(1-(3-(1H-pyrazol-1-yl)propanoyl)-1,2,5,6-tetrahydropyridin-3-yl)-4-chloro-7-fluoro-N,N-dimethyl-1H-indole-2-carboxamide (200 mg, 450 μmol, synthesized via Step 1 of Intermediate AR), tert-butyl 4-(3-methoxy-4-(4,4,5,5-tetramethyl-1,3,2-dioxaborolan-2-yl)phenyl)piperazine-1-carboxylate (226 mg, 541 μmol, Intermediate D), XPhos PdG3 (38.1 mg, 45.1 mol) and K₃PO₄ (287 mg, 1.35 mmol) in dioxane (2.00 mL) and H₂O (0.40 mL) was degassed and purged with N₂ three times. Then the mixture was stirred at 90° C. for 2 h under N₂ atmosphere. The reaction mixture was quenched with addition of aq. NH₄Cl (10.0 mL) at 25° C., and then diluted with water (10.0 mL) and extracted with EA (10 mL×4). The combined organic layers were washed with brine (20.0 mL), dried over anhydrous Na₂SO₄, filtered and concentrated under reduced pressure to give a residue. The residue was purified by flash silica gel chromatography (ISCO®; 4 g Sepa Flash® Silica Flash Column, Eluent of 0˜100% Ethyl acetate/Petroleum ether gradient @18 mL/min) to give the title compound (212 mg, 67% yield) as a yellow gum. LC-MS (ESI⁺) m/z 700.4 (M+H)⁺; ¹H NMR (400 MHz, CDCl₃) δ ppm 9.37 (d, J=7.2 Hz, 1H), 7.55-7.45 (m, 2H), 7.33-7.27 (m, 1H), 6.95 (dd, J=6.4, 17.6 Hz, 1H), 6.65 (s, 3H), 6.21 (d, J=10.0 Hz, 1H), 6.13 (d, J=14.8 Hz, 1H), 4.54 (q, J=6.4 Hz, 2H), 4.47 (s, 1H), 4.26 (s, 1H), 3.81-3.75 (m, 4H), 3.74-3.59 (m, 4H), 3.55 (t, J=5.6 Hz, 1H), 3.45-3.07 (m, 10H), 3.05-2.94 (m, 2H), 2.33 (s, 2H), 1.51 (s, 9H).

### Step 2—6-(1-(3-(1H-pyrazol-1-yl)propanoyl)-1,2,5,6-tetrahydropyridin-3-yl)-7-fluoro-4-(2-methoxy-4-(piperazin-1-yl)phenyl)-N,N-dimethyl-1H-indole-2-carboxamide

To a solution of tert-butyl 4-(4-(6-(1-(3-(1H-pyrazol-1-yl)propanoyl)-1,2,5,6-tetrahydropyridin-3-yl)-2-(dimethylcarbamoyl)-7-fluoro-1H-indol-4-yl)-3-methoxyphenyl)piperazine-1-carboxylate (212 mg, 303 μmol) in DCM (1.60 mL) was added TFA (614 mg, 5.38 mmol, 0.40 mL). The mixture was then stirred at 25° C. for 1 hr. On completion, the reaction mixture was concentrated under reduced pressure to give the title compound (459 mg, TFA) as black gum. LC-MS (ESI⁺) m/z 600.4 (M+H) ⁺.

### 2-(4-(2,6-Dioxopiperidin-3-yl)-3-fluorophenyl)acetaldehyde (Intermediate DJ)

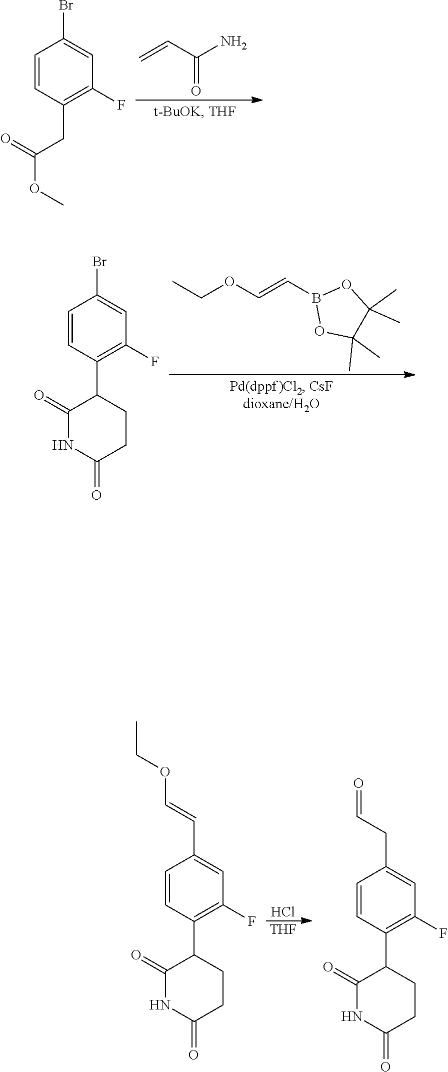

### Step 1—3-(4-Bromo-2-fluorophenyl)piperidine-2,6-dione

To a solution of methyl 2-(4-bromo-2-fluorophenyl)acetate (10.0 g, 40.5 mmol, CAS #193290-19-6), acrylamide (2.88 g, 40.5 mmol, 2.79 mL) in THF (100 mL) was added t-BuOK (1 M, 44.5 mL) at 0° C. under N₂. The mixture was then stirred at 50° C. for 2 h. On completion, the reaction mixture was quenched by addition of aq. NH₄Cl (150 mL) at 0° C., and then extracted with EA (100 mL×2) and MeCN (100 mL×2). The combined organic layers were dried with anhydrous Na₂SO₄ and concentrated under reduced pressure to give a residue. The residue was purified by flash silica gel chromatography (ISCO®; 120 g Sepa Flash® Silica Flash Column, Eluent of 0˜100% Ethyl acetate/Petroleum ether gradient @100 mL/min) to give the title compound (9.00 g, 75% yield) as a pink solid. LC-MS (ESI⁺) m/z 285.9 (M+H). ¹H NMR (400 MHz, CDCl₃) δ ppm 8.37 (s, 1H), 7.25 (d, J=2.4 Hz, 1H), 7.03 (t, J=8.0 Hz, 1H), 3.84 (dd, J=5.2, 11.6 Hz, 1H), 2.83-2.71 (m, 1H), 2.69-2.59 (m, 1H), 2.29-2.11 (m, 2H).

### Step 2—(E)-3-(4-(2-ethoxyvinyl)-2-fluorophenyl)piperidine-2,6-dione

A mixture of 3-(4-bromo-2-fluorophenyl)piperidine-2,6-dione (2.00 g, 6.99 mmol), (E)-2-(2-ethoxyvinyl)-4,4,5,5-tetramethyl-1,3,2-dioxaborolane (1.80 g, 9.09 mmol), CsF (2.12 g, 14.0 mmol, 516 μL) and Pd(dppf)Cl₂ (511 mg, 699 mol) in H₂O (2.00 mL) and dioxane (20.0 mL) was degassed and purged with N₂ three times. Then the mixture was stirred at 100° C. for 2 h under N₂ atmosphere. On completion, the reaction mixture was quenched with addition of water (100 mL) at 0° C., and then extracted with EA (50.0 mL×3). The combined organic layers were dried with anhydrous Na₂SO₄ and concentrated under reduced pressure to give a residue. The residue was purified by flash silica gel chromatography (ISCO®; 40 g Sepa Flash® Silica Flash Column, Eluent of 0˜60% Ethyl acetate/Petroleum ether gradient @80 mL/min) to give the title compound (1.73 g, 85% yield) as orange solid. LC-MS (ESI⁺) m/z 278.1 (M+H)⁺; ¹H NMR (400 MHz, CDCl₃) δ ppm 8.08 (s, 1H), 7.08-7.03 (m, 111), 7.02-6.93 (m, 3H), 5.78 (d, J=12.8 Hz, 1H), 3.96-3.84 (m, 3H), 2.84-2.74 (m, 1H), 2.73-2.61 (m, 1H), 2.35-2.24 (m, 1H), 2.24-2.16 (m, 1H), 1.35 (t, J=7.2 Hz, 3H).

### Step 3—2-(4-(2,6-Dioxopiperidin-3-yl)-3-fluorophenyl)acetaldehyde

To a solution of (E)-3-(4-(2-ethoxyvinyl)-2-fluorophenyl)piperidine-2,6-dione (700 mg, 2.52 mmol) in THF (3.50 mL) was added HCl (6 M, 421 μL). The mixture was then stirred at 25° C. for 2 hrs. On completion, the reaction mixture was quenched by addition of water (100 mL) at 0° C., and then extracted with EA (50.0 mL×3). The combined organic layers were dried with anhydrous Na₂SO₄ and concentrated under reduced pressure to give a residue. The residue was purified by flash silica gel chromatography (ISCO®; 12 g Sepa Flash® Silica Flash Column, Eluent of 0˜50% Ethyl acetate/Petroleum ether gradient a 40 mL/min) to give the title compound (287 mg, 45% yield) as a white solid. LC-MS (ESI⁺) n/z 250.1 (M+H); ¹H NMR (400 MHz, CDCl₃) δ ppm 9.77 (t, J=2.0 Hz, 1H), 8.04 (s, 1H), 7.24-7.17 (m, 1H), 7.06-6.98 (m, 2H), 3.93 (dd, J=5.2, 11.5 Hz, 1H), 3.72 (d, J=1.6 Hz, 2H), 2.88-2.77 (m, 1H), 2.75-2.64 (m, 1H), 2.37-2.20 (m, 2H).

### 2-(1-(Tert-butoxycarbonyl)-1,2,5,6-tetrahydropyridin-3-yl)-7-chloro-1H-indole-5-carboxylic acid (Intermediate DK)

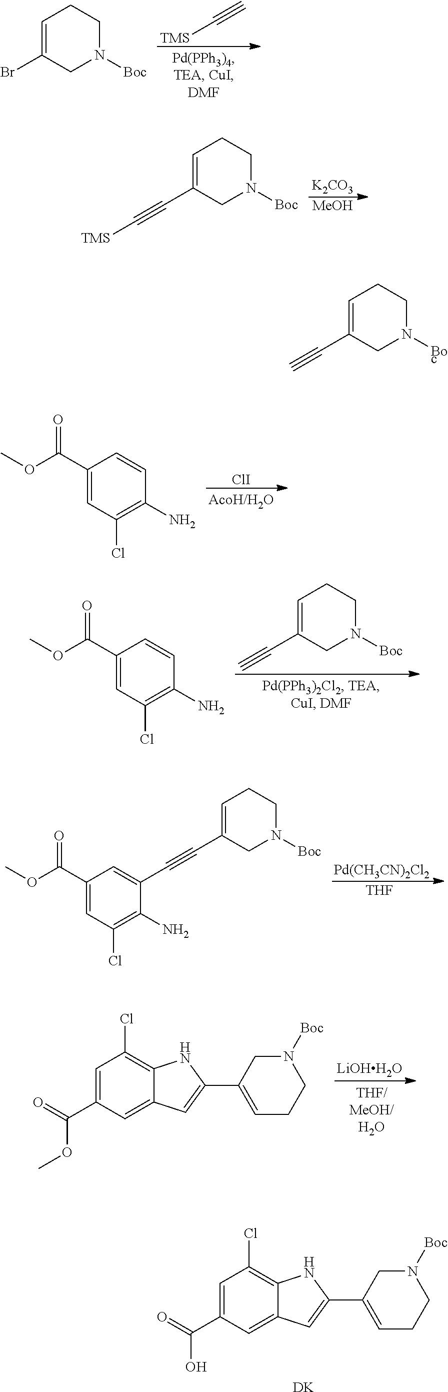

### Step 1—Tert-butyl 5-((trimethylsilyl)ethynyl)-3,6-dihydropyridine-1(2H)-carboxylate

A mixture of tert-butyl 5-bromo-3,6-dihydropyridine-1(2H)-carboxylate (1 g, 3.81 mmol, CAS #1415841-82-5), ethynyltrimethylsilane (3.75 g, 38.1 mmol, 5.28 mL, CAS #1066-54-2), Pd(PPh₃)₄ (441 mg, 381 μmol), CuI (72.7 mg, 381 μmol) and TEA (1.16 g, 11.4 mmol, 1.59 mL) in DMF (20 mL) was degassed and purged with N₂ three times. Then the mixture was stirred at 50° C. for 2 hrs under N₂ atmosphere. On completion, the reaction mixture was diluted with water (40 mL) and extracted with dichloromethane (3×40 mL). The combined organic layers were washed with brine (50 mL) and dried over anhydrous sodium sulfate, filtered and the filtrate was concentrated in vacuo to give the crude residue. The residue was purified by column chromatography (SiO₂, Petroleum ether:Ethyl acetate=1/0 to 10/1) to give the title compound (500 mg, 46% yield) as a yellow solid. ¹H NMR (400 MHz, CDCl₃-d) 8=6.25 (br s, 1H), 3.92 (br s, 2H), 3.45 (br t, J=5.6 Hz, 2H), 2.20 (br s, 2H), 1.47 (s, 9H), 0.19 (s, 9H).

### Step 2—Tert-butyl 5-ethynyl-3,6-dihydropyridine-1(2H)-carboxylate

To a solution of tert-butyl 5-((trimethylsilyl)ethynyl)-3,6-dihydropyridine-1(2H)-carboxylate (500 mg, 1.79 mmol) in MeOH (5 mL) was added K₂CO₃ (742 mg, 5.37 mmol). The mixture was then stirred at 25° C. for 2 hrs. On completion, the reaction mixture was diluted with water (10 mL) and extracted with ethyl acetate (3×10 mL). The combined organic layers were concentrated in vacuo to give the title compound (300 mg) as a yellow oil. ¹H NMR (400 MHz, CHLOROFORM-d) δ=6.29 (br s, 1H), 3.94 (br s, 2H), 3.47 (t, J=5.6 Hz, 2H), 2.87 (s, 1H), 2.21 (br s, 2H), 1.47 (s, 9H).

### Step 3—Methyl 4-amino-3-chloro-5-iodobenzoate

To a solution of methyl 4-amino-3-chlorobenzoate (11 g, 59.3 mmol) in AcOH (50 mL) and H2O (50 mL) was added chloroiodide (9.62 g, 59.3 mmol, 3.02 mL). The mixture was then stirred at 70° C. for 3 hrs. On completion, the reaction mixture was filtered and the filtrate cake was concentrated in vacuo to give the title compound (12 g) as a yellow solid. ¹H NMR (400 MHz, DMSO-d₆) δ=8.08 (d, J=2.0 Hz, 1H), 7.75 (d, J=2.0 Hz, 1H), 6.09 (s, 2H), 3.77 (s, 3H).

### Step 4—Tert-butyl 5-((2-amino-3-chloro-5-(methoxycarbonyl)phenyl)ethynyl)-3,6-dihydropyridine-1(2H)-carboxylate

A mixture of methyl 4-amino-3-chloro-5-iodobenzoate (100 mg, 321 μmol), tert-butyl 5-ethynyl-3,6-dihydropyridine-1(2H)-carboxylate (79.9 mg, 385 μmol), Pd(PPh₃)₂C1₂ (22.5 mg, 32.1 mol), CuI (6.11 mg, 32.1 μmol) and TEA (162 mg, 1.61 mmol, 223 L) in DMF (2 mL) was degassed and purged with N₂ three times. Then the mixture was stirred at 50° C. for 12 hrs under N₂ atmosphere. On completion, the reaction mixture was diluted with water (4 mL) and extracted with ethyl acetate (3×10 mL). The combined organic layers were washed with brine (20 mL) and dried over anhydrous sodium sulfate, filtered and the filtrate was concentrated in vacuo to give the crude residue. The residue was purified by column chromatography (SiO₂, Petroleum ether:Ethyl acetate 1/0 to 5/1) to give the title compound (80 mg, 64% yield) as a yellow solid. ¹H NMR (400 MHz, DMSO-d₆) δ=7.79 (dd, J=2.0, 14.4 Hz, 2H), 6.51 (br s, 1H), 6.40 (s, 2H), 4.06 (br d, J=2.0 Hz, 2H), 3.84 (s, 3H), 3.48 (br t, J=5.6 Hz, 2H), 2.28 (br d, J=4.0 Hz, 2H), 1.49 (s, 9H).

### Step 5—Methyl 2-(1-(tert-butoxycarbonyl)-1,2,5,6-tetrahydropyridin-3-yl)-7-chloro-1H-indole-5-carboxylate

A mixture of tert-butyl 5-((2-amino-3-chloro-5-(methoxycarbonyl)phenyl)ethynyl)-3,6-dihydropyridine-1(2H)-carboxylate (4 g, 10.2 mmol) and Pd(CH₃CN)₂Cl₂ (1.33 g, 5.12 mmol) in THF (30 mL) was degassed and purged with N₂ three times. Then the mixture was stirred at 80° C. for 1 hr under N₂ atmosphere. On completion, the reaction mixture was diluted with water (50 mL) and extracted with dichloromethane (3×50 mL). The combined organic layers were washed with brine (50 mL) and dried over anhydrous sodium sulfate, filtered and the filtrate was concentrated in vacuo to give the title compound (4 g) as a yellow solid. ¹H NMR (400 MHz, DMSO-d₆) δ=11.65 (s, 1H), 8.20-8.12 (m, 1H), 7.69 (d, J=1.2 Hz, 1H), 6.82 (br s, 1H), 6.69 (br s, 1H), 4.25 (br s, 2H), 3.85 (s, 3H), 3.49 (brt, J=5.6 Hz, 2H), 2.30 (br s, 2H), 1.44 (s, 9H).

### Step 6—2-(1-(Tert-butoxycarbonyl)-1,2,5,6-tetrahydropyridin-3-yl)-7-chloro-1H-indole-5-carboxylic acid

To a solution of tert-butyl 5-((2-amino-3-chloro-5-(methoxycarbonyl)phenyl)ethynyl)-3,6-dihydropyridine-1(2H)-carboxylate (4 g, 10.2 mmol) in THF (20 mL), MeOH (5 mL) and H₂O (5 mL) was added LiOH·H₂O (1.72 g, 40.9 mmol). The mixture was then stirred at 25° C. for 12 hrs. On completion, HCl(1 M) was added to the mixture until the pH was 6.0. The mixture was extracted with ethyl acetate (3×20 mL). The combined organic layers were dried over anhydrous sodium sulfate, filtered and the filtrate was concentrated in vacuo to give the title compound (3.5 g) as a white solid. LC-MS (ESI⁺) m/z 377.0 (M+H)⁺.

### Tert-butyl 4-(4-chloro-3-methoxyphenyl)piperidine-1-carboxylate (Intermediate DL)

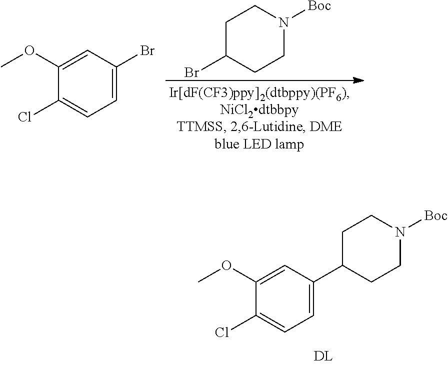

A mixture of 4-bromo-1-chloro-2-methoxybenzene (1 g, 4.52 mmol, 613 μL, CAS #16817-43-9), tert-butyl 4-bromopiperidine-1-carboxylate (1.43 g, 5.42 mmol) bis[3,5-difluoro-2-[5-(trifluoromethyl)-2-pyridyl]phenyl]iridium(1+) 4-tert-butyl-2-(4-tert-butyl-2-pyridyl)pyridine hexafluorophosphate (50.7 mg, 45.2 μmol, CAS #180695-79-8), 4-tert-butyl-2-(4-tert-butyl-2-pyridyl)pyridine dichloronickel (26.9 mg, 67.7 μmol), TTMSS (1.12 g, 4.52 mmol, 1.39 mL) and 2,6-dimethylpyridine (4.35 g, 40.6 mmol, 4.73 mL) in anhydrous DME (30 mL) was degassed and purged with nitrogen for three times. Then the reaction was stirred and irradiated with a 4×50 W [455 nm] blue LED lamp (3 cm away), with cooling water to keep the reaction temperature at 25° C. for 6 hrs under nitrogen atmosphere. On completion, the mixture was diluted with H₂O (50 mL) and adjusted to pH of 2 by adding IM HCl solution, then extracted with DCM (50 mL×3), concentrated to give a crude product. The residue was purified by column chromatography (SiO₂. Petroleum ether:Ethyl acetate=50/1 to 10/1) to give the title compound (0.8 g, 54% yield) as a white solid. LC-MS (ESI⁺) m/z 270.1 (M-55)⁺.

### 7-fluoro-4-[2-methoxy-4-(4-piperidyl)phenyl]-N,N-dimethyl-6-[1-[3-(triazol-1-yl)propanoyl]-3,6-dihydro-2H-pyridin-5-yl]-1H-indole-2-carboxamide (Intermediate DM)

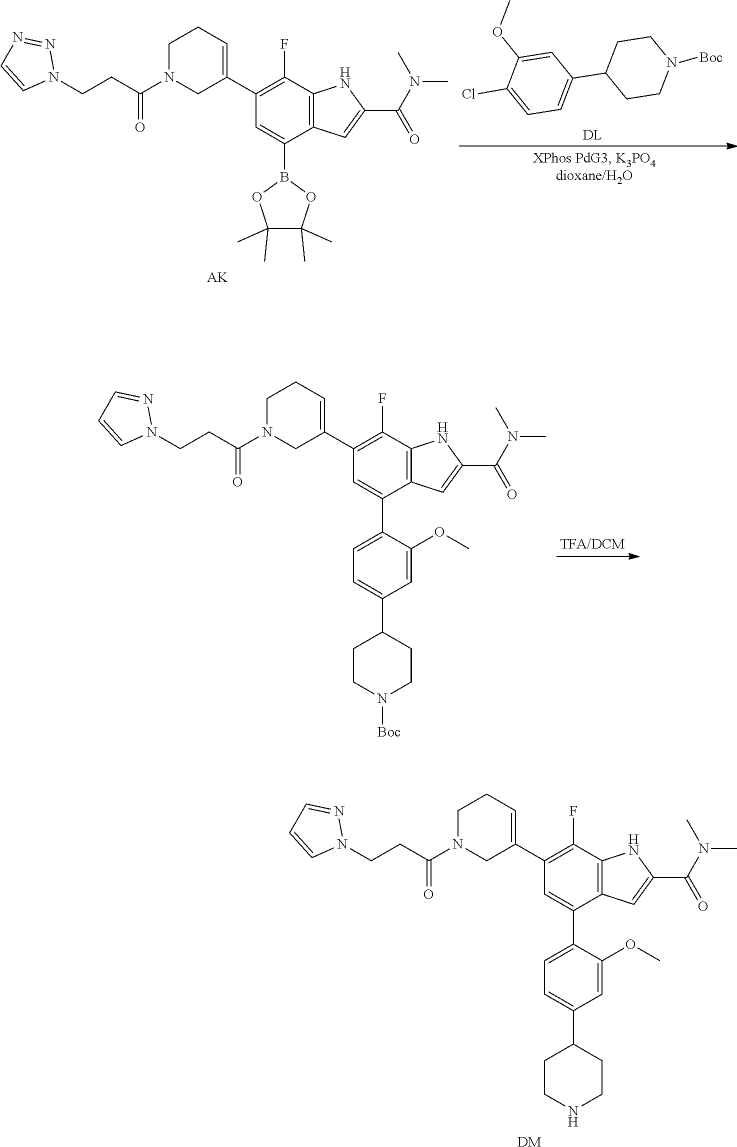

### Step 1—Tert-butyl 4-[4-[2-(dimethylcarbamoyl)-7-fluoro-6-[1-[3-(triazol-1-yl)p ropanoyl]-3,6-dihydro-2H-pyridin-5-yl]-1H-indol-4-yl]-3-methoxy-phenyl]piperidine-1-carboxylate

A mixture of tert-butyl 4-(4-chloro-3-methoxy-phenyl)piperidine-1-carboxylate (182 mg, 559 μmol, Intermediate DL), 7-fluoro-N,N-dimethyl-4-(4,4,5,5-tetramethyl-1,3,2-dioxaborolan-2-yl)-6-[1-[3-(triazol-1-yl)propanoyl]-3,6-dihydro-2H-pyridin-5-yl]-1H-indole-2-carboxamide (300 mg, 559 μmol, Intermediate AK), XPhos Pd G3 (47 mg, 55.9 μmol), and K₃PO₄ (356 mg, 1.68 mmol) in dioxane (3.0 mL) and H₂O (0.3 mL) was degassed and purged with N₂ three times. Then the mixture was stirred at 85° C. for 1 hr under N₂ atmosphere. On completion, the mixture was diluted with water (50 mL) and extracted with ethyl acetate (50 mL×2), dried over sodium sulfate, filtered and concentrated under reduced pressure. The residue was purified by silica gel column chromatography (petroleum ether/ethyl acetate=3/1˜ethyl acetate) to give the title compound (300 mg, 69% yield) as a white solid. LC-MS (ESI⁺) m/z 700.3 (M+H) ⁺.

### Step 2—7-Fluoro-4-[2-methoxy-4-(4-piperidyl)phenyl]-N,N-dimethyl-6-[1-[3-(triazol-1-yl)propanoyl]-3,6-dihydro-2H-pyridin-5-yl]-1H-indole-2-carboxamide

To a solution of tert-butyl 4-[4-[2-(dimethylcarbamoyl)-7-fluoro-6-[1-[3-(triazol-1-yl)propanoyl]-3,6-dihydro-2H-pyridin-5-yl]-1H-indol-4-yl]-3-methoxy-phenyl]piperidine-1-carboxylate (300 mg, 428 μmol) in DCM (3.0 mL) was added TFA (1.23 g, 10.7 mmol, 0.8 mL). The mixture was stirred at 20° C. for 0.5 h. On completion, the mixture was concentrated under reduced pressure to give the title compound (500 mg, 4TFA) as a yellow oil. LC-MS (ESI⁺) m/z 600.2 (M+H)⁺.

### 3-(3-Methyl-2-oxo-4-(3-oxocyclobutyl)-2,3-dihydro-1H-benzo[d]imidazol-1-yl)piperidine-2,6-dione (Intermediate DN)

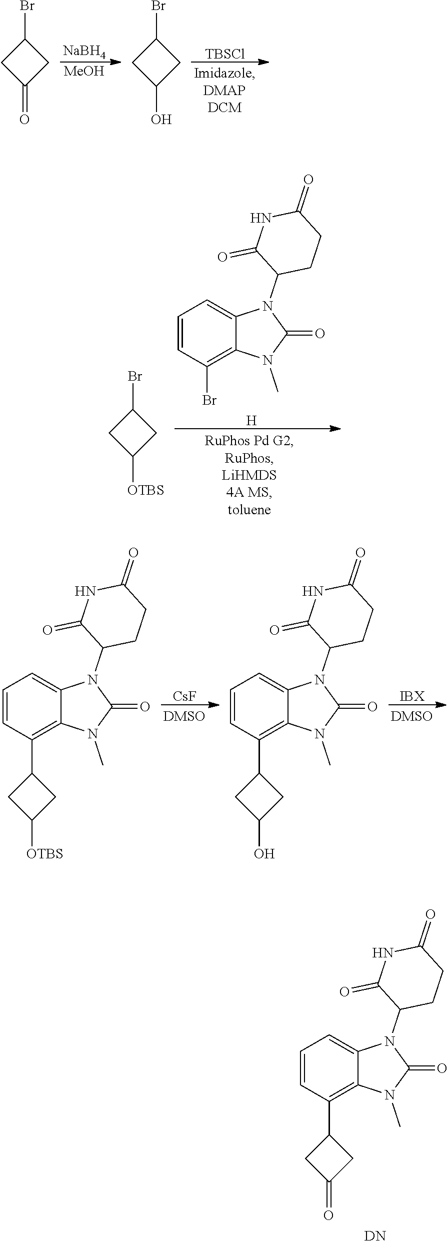

### Step 1—3-Bromocyclobutan-1-ol

To a solution of 3-bromocyclobutanone (2 g, 13.4 mmol, CAS #23761-24-2) in MeOH (25 mL) was added NaBH₄ (1.27 g, 33.6 mmol) at 0° C. The mixture was then stirred at 20° C. for 2 hrs. On completion, the reaction mixture was quenched by addition of sat. NH₄Cl 20 mL at 0° C., and then diluted with H₂O (10 mL) and extracted with EA (20 mL×4). The combined organic layers dried over Na₂SO₄, filtered and concentrated under reduced pressure to give the title compound (1.8 g) as a colorless oil. ¹H NMR (400 MHz, CHLOROFORM-d) δ=4.28 (q, J=7.2 Hz, 1H), 4.06 (q, J=7.2 Hz, 1H), 3.94-3.82 (m, 1H), 3.06-2.88 (m, 2H), 2.46-2.30 (m, 2H).

### Step 2—(3-Bromocyclobutoxy)(tert-butyl)dimethylsilane

To a solution of 3-bromocyclobutanol (1 g, 6.62 mmol) in DCM (12 mL) was added imidazole (902 mg, 13.2 mmol), DMAP (80.9 mg, 662 μmol) and TBSCl (1.50 g, 9.93 mmol, 1.22 mL). The mixture was then stirred at 25° C. for 12 hrs. On completion, the reaction mixture was diluted with H₂O (20 mL) and extracted with DCM (20 mL×3). The combined organic layers were dried over Na₂SO₄, filtered and concentrated under reduced pressure to give a residue. The residue was purified by column chromatography (SiO₂, Petroleum ether/Ethyl acetate=1/0) to give the title compound (1.43 g, 81% yield) as a colorless oil. ¹H NMR (400 MHz, CHLOROFORM-d) δ=4.07-3.97 (m, 1H), 3.87 (tt, J=7.2, 8.8 Hz, 1H), 2.98-2.87 (m, 2H), 2.50-2.37 (m, 2H), 0.85 (s, 8H), 0.03-0.02 (m, 6H).

### Step 3—3-(4-(3-((Tert-butyldimethylsilyl)oxy)cyclobutyl)-3-methyl-2-oxo-2,3-dihydro-1H-benzo[d]imidazol-1-yl)piperidine-2,6-dione

To an 15 mL vial equipped with a stir bar was added 3-(4-bromo-3-methyl-2-oxo-benzimidazol-1-yl)piperidine-2,6-dione (0.7 g, 2.07 mmol. Intermediate H). (3-bromocyclobutoxy)-tert-butyl-dimethyl-silane (604 mg, 2.28 mmol), Ir[dF(CF₃)ppy]₂(dtbpy)(PF₆) (46.5 mg, 41.4 μmol), NiCl₂·dtbbpy (24.7 mg, 62.1 mol), TTMSS (515 mg, 2.07 mmol), and 2,6-lutidine (2.00 g, 18.6 mmol, 2.17 mL) in DME (10 mL). The vial was sealed and placed under nitrogen was added. The reaction was stirred and irradiated with a 10 W blue LED lamp (3 cm away), with cooling water to keep the reaction temperature at 25° C. for 14 hrs. On completion, the reaction mixture was concentrated under reduced pressure to give a residue The residue was purified by column chromatography (SiO₂, DCM/EA=1/0 to 1/1) to give the title compound (330 mg, 35% yield) as a yellow solid. LC-MS (ESI⁺) m/z 444.1 (M+H)⁺.

### Step 4—3-(4-(3—Hydroxycyclobutyl)-3-methyl-2-oxo-2,3-dihydro-1H-benzo[d]imidazol-1-yl)piperidine-2,6-dione

To a solution of 3-[4-[3-[tert-butyl(dimethyl)silyl]oxycyclobutyl]-3-methyl-2-oxo-benzimidazol-1-yl]piperidine-2,6-dione (330 mg, 744 μmol) in DMSO (4 mL) was added CsF (1.13 g, 7.44 mmol). The mixture was then stirred at 50° C. for 1 hr. On completion, FA (1 mL) was added to the reaction mixture, then the mixture was concentrated in vacuo to give the residue. The residue was purified by prep-HPLC (0.1% FA condition) to give the title compound (187 mg, 75% yield) as a white solid. LC-MS (ESI⁺) m/z 330.1 (M+H)⁺.

### Step 5—3-(3-Methyl-2-oxo-4-(3-oxocyclobutyl)-2,3-dihydro-1H-benzo[d]imidazol-1-yl)piperidine-2,6-dione

To a solution of 3-[4-(3-hydroxycyclobutyl)-3-methyl-2-oxo-benzimidazol-1-yl]piperidine-2,6-dione (167 mg, 5076 μmol) in DMSO (1 mL) was added IBX (284 mg, 1.01 mmol). The mixture was then stirred at 25° C. for 2 hrs. On completion, the reaction mixture was concentrated under reduced pressure to give a residue. The residue was purified by prep-HPLC (0.1% FA condition) to give the title compound (162 mg, 97% yield) as a white solid. LC-MS (ESI⁺) m/z 328.0 (M+H)⁺. ¹H NMR (400 MHz, DMSO-d₆) δ=11.09 (s, 1H), 7.19 (dd, J=2.0, 6.8 Hz, 1H), 7.10-7.00 (m, 2H), 5.38 (dd, J=5.6, 12.4 Hz, 1H), 4.41-4.28 (m, 1H), 3.60 (s, 3H), 3.53-3.43 (m, 2H), 3.40-3.33 (m, 2H), 2.95-2.83 (m, 1H), 2.77-2.67 (m, 1H), 2.65-2.59 (m, 1H), 2.05-1.94 (m, 1H).

### 3-(4-(3-(4-(4-Chlorophenyl)piperazin-1-yl)cyclobutyl)-3-methyl-2-oxo-2,3-dihydro-1H-benzo[d]imidazol-1-yl)piperidine-2,6-dione (Intermediate DO)

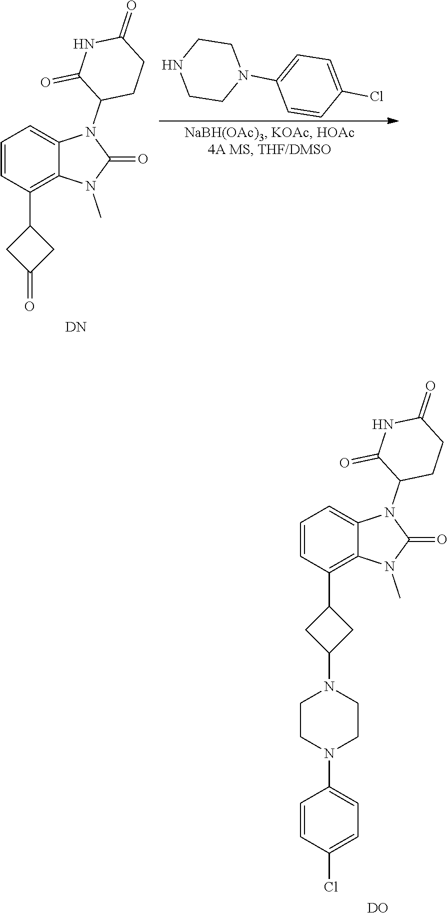

To a solution of 3-[3-methyl-2-oxo-4-(3-oxocyclobutyl)benzimidazol-1-yl]piperidine-2,6-dione (162 mg, 495 μmol, Intermediate DN) and 1-(4-chlorophenyl)piperazine (195 mg, 990 μmol, CAS #38212-33-8) in THF (2 mL) and DMSO (0.5 mL) was added KOAc (146 mg, 1.48 mmol) and HOAc (149 mg, 2.47 mmol) at 20° C. After addition, the mixture was stirred at 20° C. for 12 hrs. and then NaBH₃CN (93.30 mg, 1.48 mmol) was added at 0° C. The resulting mixture was stirred at 0° C. for 0.5 hr. On completion, FA (2 mL) was added to the reaction mixture. The residue was then purified by prep-HPLC (0.1% FA condition) to give the title compound (150 mg, 55% yield) as a white solid. LC-MS (ESI⁺) m/z 508.2 (M+H)⁺; ¹H NMR (400 MHz, DMSO-d₆) δ=11.10 (s, 1H), 7.23 (d, J=8.8 Hz, 2H), 7.10-6.98 (m, 3H), 6.95 (d, J=9.2 Hz, 2H), 5.37 (dd, J=5.2, 12.8 Hz, 1H), 3.15 (s, 4H), 2.92-2.77 (m, 2H), 2.74-2.61 (m, 3H), 2.48 (s, 6H), 2.08-1.95 (m, 3H).

### 4-(Azetidin-3-yl)-1-(4-chlorophenyl)piperidine (Intermediate DP)

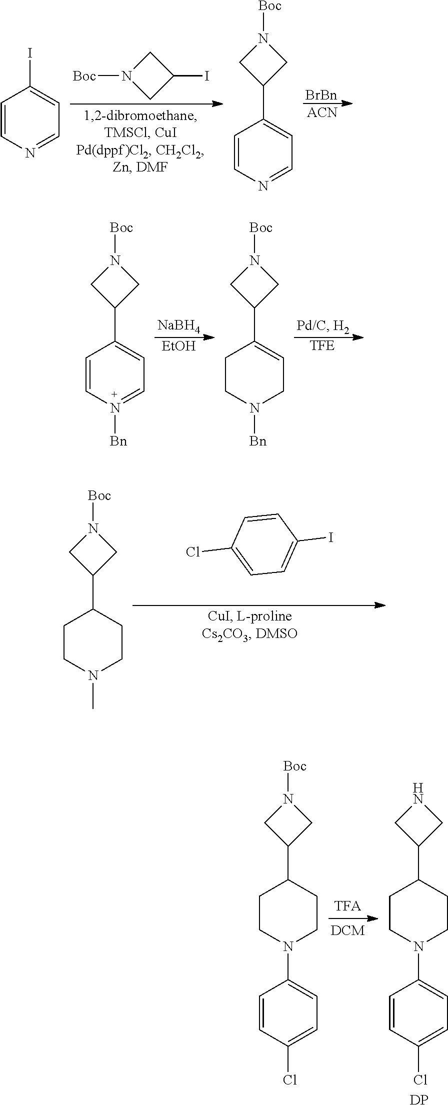

### Step 1—Tert-butyl 3-(pyridin-4-yl)azetidine-1-carboxylate

A 3-neck round bottom flask was charged with anhydrous DMF (50 mL) and Zn (4.78 g, 73.1 mmol). The mixture was then stirred at 20° C. Then, a mixture of 1,2-dibromomethane (916 mg, 4.88 mmol) and TMSCl (529 mg, 4.88 mmol) was added at a rate to maintain the temperature below 65° C. for 30 min. The resulting slurry was stirred for 30 min. Then, a solution of tert-butyl 3-iodoazetidine-1-carboxylate (9.39 g, 33.1 mmol. CAS #254454-54-1) in DMF (50 mL) was added dropwise over 10 min at a rate to maintain the temperature below 65° C. and the milky suspension was stirred for 30 min while slowly cooling to 20° C. Another round bottom flask was charged with Pd(dppf)Cl₂·CH₂Cl₂ (199 mg, 243 mol), CuI (139 mg, 731 μmol) and 4-iodopyridine (5 g, 24.3 mmol, CAS #15854-87-2) in DMF (50 mL) under N₂. The resulting mixture was degassed with alternate vacuum/N₂ purges. The above prepared zinc iodide reagent of compound in DMF was added as a suspension. The mixture was degassed with vacuum/N₂ twice and then heated to 80° C. for 4 h. On completion, the reaction mixture was filtered and concentrated under reduced pressure. The filter cake was quenched with 1N HCl solution (100 mL). The crude product was poured into H₂O (200 mL). The mixture was extracted with ethyl acetate (250 mL×3). The organic phase was washed with brine (100 mL), dried over anhydrous sodium sulfate, and concentrated in vacuo to give a residue. The crude product was purified by flash silica gel chromatography (ISCO®; 80 g SepaFlash® Silica Flash Column, Eluent of 0˜45% Ethyl acetate/Petroleum ether gradient @, 120 mL/min) to give a title compound (5.0 g) as an orange solid. ¹H NMR (400 MHz, DMSO-d6) δ=8.54 (s, 2H), 7.36 (d, J=4.8 Hz, 2H), 4.31-4.18 (m, 2H), 3.89-3.77 (m, 3H), 1.40 (s, 9H).

### Step 2—1-Benzyl-4-(1-(tert-butoxycarbonyl)azetidin-3-yl)pyridin-1-ium

To a solution of tert-butyl 3-(pyridin-4-yl)azetidine-1-carboxylate (5.3 g, 22.6 mmol) in ACN (20 mL) was added (bromomethyl)benzene (3.99 g, 23.3 mmol). The mixture was then stirred at 80° C. for 2 hrs. On completion, the reaction mixture was concentrated under vacuum. The crude product was triturated with PE (100 mL×3) at 25° C. for 30 min to give a title compound (8 g, 86% yield) as an orange solid. LC-MS (ESI⁺) m/z 326.2 (M+H)⁺.

### Step 3—Tert-butyl 3-(1-benzyl-1,2,3,6-tetrahydropyridin-4-yl)azetidine-1-carboxylate

To a solution of 1-benzyl-4-(1-(tert-butoxycarbonyl)azetidin-3-yl)pyridin-1-ium (9.5 g, 29.1 mmol) in EtOH (90 mL) was added NaBH₄ (1.88 g, 49.6 mmol) at 0° C. Then, the mixture was degassed and purged with N₂ for 3 times. The mixture was stirred at 20° C. for 3 hrs under N₂ atmosphere. On completion, the reaction mixture was quenched with saturated NH₄Cl solution (100 mL). The aqueous layer was extracted with EA (150 mL×3). The organic layer was washed with brine (100 mL), dried over anhydrous sodium sulfate and filtered. The mother liquid was concentrated. The residue was then purified by prep-HPLC (FA condition) to give a title compound (6.7 g, 67% yield) as a yellow oil. LC-MS (ESI⁺) m/z 329.3 (M+H)⁺.

### Step 4—Tert-butyl 3-(piperidin-4-yl)azetidine-1-carboxylate

To a solution of tert-butyl 3-(1-benzyl-3,6-dihydro-2H-pyridin-4-yl)azetidine-1-carboxylate (1.7 g, 5.18 mmol) in TFE (10 mL) was added Pd/C (1.5 g, 1.41 mmol, 10 wt %) under N₂ atmosphere. The suspension was degassed and purged with H₂ three times. The mixture was then stirred under H₂ (15 Psi) at 25° C. for 12 hrs. On completion, the reaction was filtered through kieselguhr very carefully, then the filtrate was concentrated in vacuo to give the title compound (1.3 g) as a white solid. LC-MS (ESI⁺) m/z 241.3 (M+H)⁺.

### Step 5—Tert-butyl 3-(1-(4-chlorophenyl)piperidin-4-yl)azetidine-1-carboxylate

A mixture of tert-butyl 3-(4-piperidyl)azetidine-1-carboxylate (1.15 g, 4.78 mmol), 1-chloro-4-iodo-benzene (1.37 g, 5.74 mmol, CAS #637-87-6), t-BuONa (2.30 g, 23.9 mmol), Pd₂(dba)₃ (438 mg, 478 μmol) and Xantphos (276 mg, 478 μmol) in toluene (25 mL) was degassed and purged with N₂ three times. Then the mixture was stirred at 100° C. for 2 hrs under N₂ atmosphere. On completion, the reaction mixture was filtered and the filtrate was concentrated in vacuo to give the crude residue. The residue was purified by column chromatography (SiO₂, Petroleum ether/Ethyl acetate=1/1 to 1/1) to give a title compound (790 mg, 44% yield) as a yellow solid. LC-MS (ESI⁺) m/z 295.1 (M+H)⁺.

### Step 6—4-(Azetidin-3-yl)-1-(4-chlorophenyl)piperidine

To a solution of tert-butyl 3-(1-(4-chlorophenyl)piperidin-4-yl)azetidine-1-carboxylate (790 mg, 2.25 mmol) in DCM (10 mL) was added TFA (2 mL). The mixture was then stirred at 25° C. for 2 hrs. On completion, the reaction mixture was added NaHCO₃ until the pH=7, then diluted by water (100 mL) and extracted with dichloromethane (3×60 mL). The combined organic layers were dried over anhydrous sodium sulfate, filtered and the filtrate was concentrated in vacuo to give the crude residue. The residue was purified by prep-HPLC (neutral condition) to give the title compound (430 mg, 76% yield) as a yellow solid. LC-MS (ESI⁺) m/z 251.0 (M+H)⁺.

### 3-(4-(3-(1-(4-chlorophenyl)piperidin-4-yl)azetidin-1-yl)-3-methyl-2-oxo-2,3-dihydro-1H-benzo[d]imidazol-1-yl)piperidine-2,6-dione (Intermediate DQ)

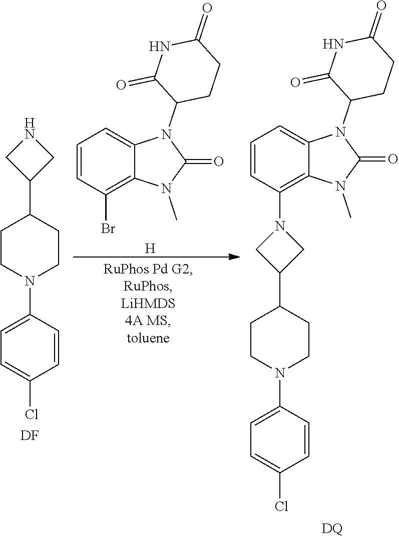

A mixture of 4-(azetidin-3-yl)-1-(4-chlorophenyl)piperidine (180 mg, 717 μmol, Intermediate DP), 3-(4-bromo-3-methyl-2-oxo-2,3-dihydro-1H-benzo[d]imidazol-1-yl)piperidine-2,6-dione (242 mg, 717 μmol, Intermediate H), Cs₂CO₃ (701 mg, 2.15 mmol), Pd₂(dba)₃ (65.7 mg, 71.7 μmol) and RuPhos (66.9 mg, 143 μmol) in dioxane (2 mL) was degassed and purged with N₂ three times. Then the mixture was stirred at 100° C. for 5 hrs under N₂ atmosphere. On completion, the reaction mixture was diluted with H₂O (50) mL and extracted with EA (50 mL×3). The combined organic layers were washed with brine (100 mL), dried over anhydrous sodium sulfate, filtered and concentrated under reduced pressure to give a residue. The residue was purified by prep-HPLC (neutral condition) to give the title compound (80 mg, 15% yield) as a yellow solid. LC-MS (ESI⁺) m/z 508.2 (M+H)⁺.

### N-formyl-2-(1-methyl-7-(2-oxoethyl)-1H-indazol-3-yl)propenamide (Intermediate DR)

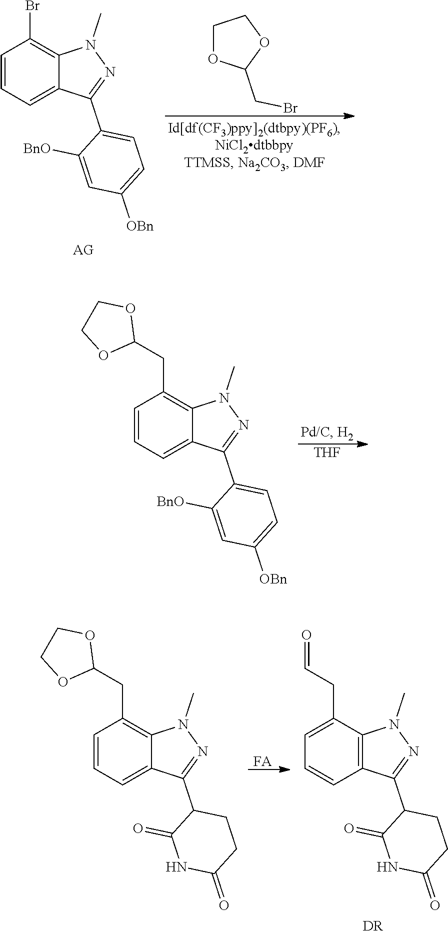

### Step 1—7-((1,3-Dioxolan-2-yl)methyl)-3-(2,6-bis(benzyloxy)pyridin-3-yl)-1-methyl-1H-indazole

To a solution of 7-bromo-3-(2,6-dibenzyloxy-3-pyridyl)-1-methyl-indazole (1.5 g, 3.00 mmol, Intermediate AG) and 2-(bromomethyl)-1,3-dioxolane (601 mg, 3.60 mmol, CAS #4360-63-8) in DME (20 mL) was added bis[3,5-difluoro-2-[5-(trifluoromethyl)-2-pyridyl]phenyl]iridium(1+); 4-tert-butyl-2-(4-tert-butyl-2-pyridyl)pyridine; hexafluorophosphate (67.3 mg, 59.9 μmol), 4-tert-butyl-2-(4-tert-butyl-2-pyridyl)pyridine dichloronickel (35.8 mg, 89.9 μmol), bis(trimethylsilyl)silyl-trimethyl-silane (745 mg, 3.00 mmol) and 2,6-dimethylpyridine (2.89 g, 27.0 mmol). The vial was sealed and placed under nitrogen was added. The reaction was then stirred and irradiated with a 10 W blue LED lamp (3 cm away), with cooling water to keep the reaction temperature at 25° C. for 14 hrs. On completion, the reaction mixture was filtered and concentrated in vacuo to give the crude residue. The crude product was purified by reverse phase HPLC (0.1% FA condition) to give the title compound (1 g, 64% yield) as a white solid. LC-MS (ESI⁺) m/z 508.3 (M+H)⁺.

### Step 2—3-(7-((1,3-Dioxolan-2-yl)methyl)-1-methyl-1H-indazol-3-yl)piperidine-2,6-dione

To a solution of 3-(2,6-dibenzyloxy-3-pyridyl)-7-(1,3-dioxolan-2-ylmethyl)-1-methyl-indazole (750 mg, 1.48 mmol) in THF (30 mL) was added Pd/C (750 mg, 10 wt %) under N₂ atmosphere. The suspension was degassed and purged with H₂ three times. Then the mixture was stirred under H₂ (40 Psi) at 30° C. for 12 hrs. On completion, the reaction was filtered through celite very carefully, the filtrated was concentrated under reduced pressure to give a residue. The crude product was purified by reverse phase HPLC (0.1% FA condition) to give the title compound (0.3 g, 57% yield) as a white solid. LC-MS (ESI⁺) m/1z 329.9 (M+H)⁺.

### Step 3—N-formal-2-(1-methyl-7-(2-oxoethyl)-1H-indazol-3-yl)propanamide

A solution of 3-[7-(1,3-dioxolan-2-ylmethyl)-1-methyl-indazol-3-yl]piperidine-2,6-dione (280 mg, 850 mol) in HCO₂H (4 mL) was stirred at 60° C. for 2 hrs. On completion, the reaction mixture was concentrated in vacuo to give the title compound (240 mg) as a white solid. LC-MS (ESI⁺) m/z 286.0 (M+H)⁺.

### 7-(4-Bromophenyl)-2,7-diazaspiro[3.5]nonane (Intermediate DS)

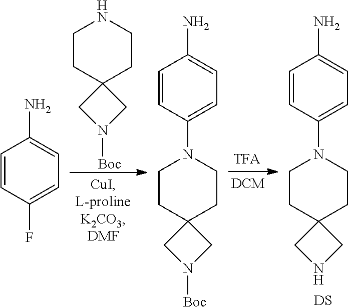

### Step 1—Tert-butyl 7-(4-bromophenyl)-2,7-diazaspiro[3.5]nonane-2-carboxylate

A mixture of tert-butyl 2,7-diazaspiro[3.5]nonane-2-carboxylate (2.00 g, 8.84 mmol, CAS #236406-55-6), 1-bromo-4-iodo-benzene (3.75 g, 13.3 mmol, CAS #589-87-7), CuI (337 mg, 1.77 mmol), (2S)-pyrrolidine-2-carboxylic acid (407 mg, 3.53 mmol) and K₂CO₃ (2.44 g, 17.7 mmol) in DMF (50 mL) was degassed and purged with N₂ three times. Then the mixture was stirred at 50° C. for 12 hrs under N₂ atmosphere. On completion, the reaction mixture was diluted with H₂O (100 mL) and extracted with EA (100 mL×3). The combined organic layers were washed with NaCl (100 mL), dried over Na₂SO₄, filtered and concentrated under reduced pressure to give a residue. The residue was purified by silica gel column chromatography (petroleum ether/ethyl acetate=50/1˜10/1) to give the title compound (1.50 g, 39% yield) as a yellow oil. LC-MS (ESI⁺) m/z 381.1 (M+H)⁺.

### Step 2—7-(4-Bromophenyl)-2,7-diazaspiro[3.5]nonane

To a solution of tert-butyl 7-(4-bromophenyl)-2,7-diazaspiro[3.5]nonane-2-carboxylate (0.5 g, 1.31 mmol) in DCM (4 mL) was added TFA (1.54 g, 13.46 mmol). Then the mixture was stirred at 25° C. for 1 hr. On completion, the mixture was filtered and concentrated under reduce pressure to give a residue to give the title compound (370 mg) as a red oil. LC-MS (ESI⁺) m/z 281.1 (M+H)⁺.

### 4-(7-(4-Bromophenyl)-2,7-diazaspiro[3.5]nonan-2-yl)-3-chloro-N-(2,6-dioxopiperidin-3-yl)benzamide (Intermediate DT)

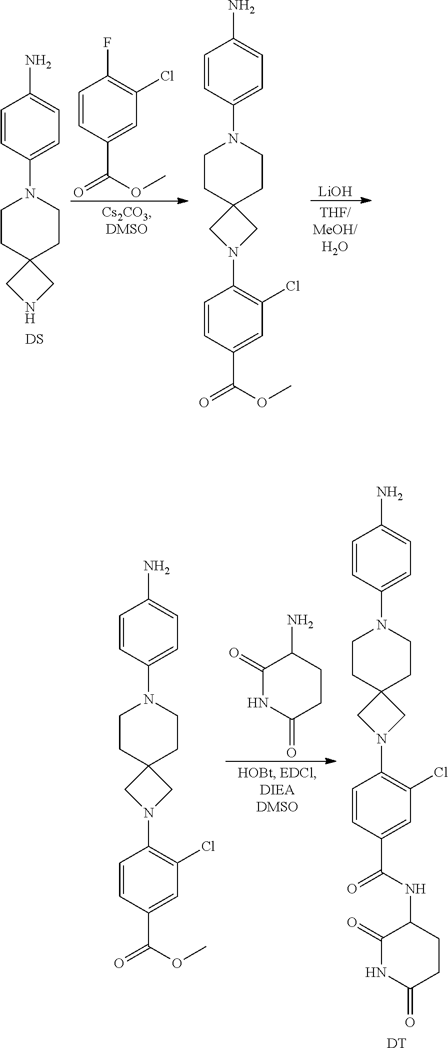

### Step 1-Methyl 4-(7-(4-bromophenyl)-2,7-diazaspiro[3.5]nonan-2-y -3-chlorobenzoate

To a solution of 7-(4-bromophenyl)-2,7-diazaspiro[3.5]nonane (810 mg, 2.88 mmol, Intermediate DS), methyl 3-chloro-4-fluoro-benzoate (815 mg, 4.32 mmol, CAS #234082-35-0) in DMSO (15 mL) was added Cs₂CO₃ (4.69 g, 14.4 mmol), then the mixture was stirred at 100° C. for 12 hr. On completion, the mixture was filtered to give a solution, then the solution was purified by reverse phase HPLC (0.1% FA) to give the title compound (550 mg, 37% yield) as a white solid. LC-MS (ESI⁺) m/z 449.1 (M+H)⁺.

### Step 2—4-(7-(4-Bromophenyl)-2,7-diazaspiro[3.5]nonan-2-yl)-3-chlorobenzoic acid

To a solution of methyl 4-[7-(4-bromophenyl)-2,7-diazaspiro[3.5]nonan-2-yl]-3-chloro-benzoate (550 mg, 1.22 mmol) in H₂O (3 mL), MeOH (3 mL) and THF (3 mL) was added LiOH·H₂O (257 mg, 6.11 mmol). The mixture was then stirred at 25° C. for 1 hr. On completion, the mixture was quenched by 2M HCl (5 mL) at 25° C. until the pH=5-6. Then concentrated under reduce pressure to give a residue to give the title compound (900 mg) as a yellow oil. LC-MS (ESI⁺) m/z=435.0 (M+H)⁺.

### Step 3—4-(7-(4-Bromophenyl)-2,7-diazaspiro[3.5]nonan-2-yl)-3-chloro-N-(2,6-dioxopiperidin-3-yl)benzamide

To a solution of 4-[7-(4-bromophenyl)-2,7-diazaspiro[3.5]nonan-2-yl]-3-chloro-benzoic acid (0.7 g, 1.61 mmol) in DMSO (10 mL) was added EDCI (770 mg, 4.02 mmol), HOBt (326 mg, 2.41 mmol), DIEA (623 mg, 4.82 mmol), and 3-aminopiperidine-2,6-dione (206 mg, 1.61 mmol, CAS #2353-44-8). The mixture was stirred at 25° C. for 12 hrs. On completion, the mixture was filtered and concentrated under reduce pressure to give a residue. The mixture was purified by reversed phase HPLC (0.1% FA) to give the title compound (450 mg, 40% yield) as a white solid. LC-MS (ESI⁺) m/z 544.9 (M+H)⁺.

### Tert-butyl 4-(4-chloro-3-methoxyphenyl)-3-oxopiperazine-1-carboxylate (Intermediate DU)

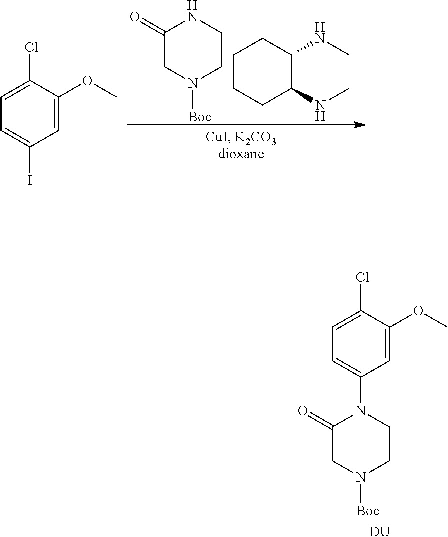

A mixture of 1-chloro-4-iodo-2-methoxybenzene (1.00 g, 3.72 mmol, CAS #161949-50-4), tert-butyl 3-oxopiperazine-1-carboxylate (746 mg, 3.72 mmol, CAS #76003-29-7), K₂CO₃ (1.03 g, 7.45 mmol), CuI (35.5 mg, 186 μmol) and (1S,2S)—N1,N2-dimethylcyclohexane-1,2-diamine (53 mg, 372 μmol) in dioxane (10.0 mL) was degassed and purged with N₂ three times. Then the mixture was stirred at 120° C. for 18 hr under N₂ atmosphere. On completion, the reaction mixture was concentrated under reduced pressure to give a residue. The residue was purified by flash silica gel chromatography (ISCO®; 40 g SepaFlash® Silica Flash Column, Eluent of 0-40% Ethyl acetate/Petroleum ether gradient a 100 mL/min) to give the title compound (1.20 g, 94% yield) as a yellow gum. LC-MS (ESI⁻) m/z 363.0 (M+Na); ¹H NMR (400 MHz, CDCl₃) δ ppm 7.39 (d, J=8.4 Hz, 1H), 6.93 (d, J=2.0 Hz, 1H), 6.80 (dd. J=2.4, 8.4 Hz, 1H), 4.26 (s, 2H), 3.93-3.89 (m, 3H), 3.83-3.77 (m, 2H), 3.76-3.69 (m, 2H), 1.51 (s, 9H).

### 6-(1-(3-(1H-1,2,3-triazol-1-yl)propanoyl)-1,2,5,6-tetrahydropyridin-3-yl)-7-fluoro-4-(2-methoxy-4-(2-oxopiperazin-1-yl)phenyl)-N,N-dimethyl-1H-indole-2-carboxamide (Intermediate DV)

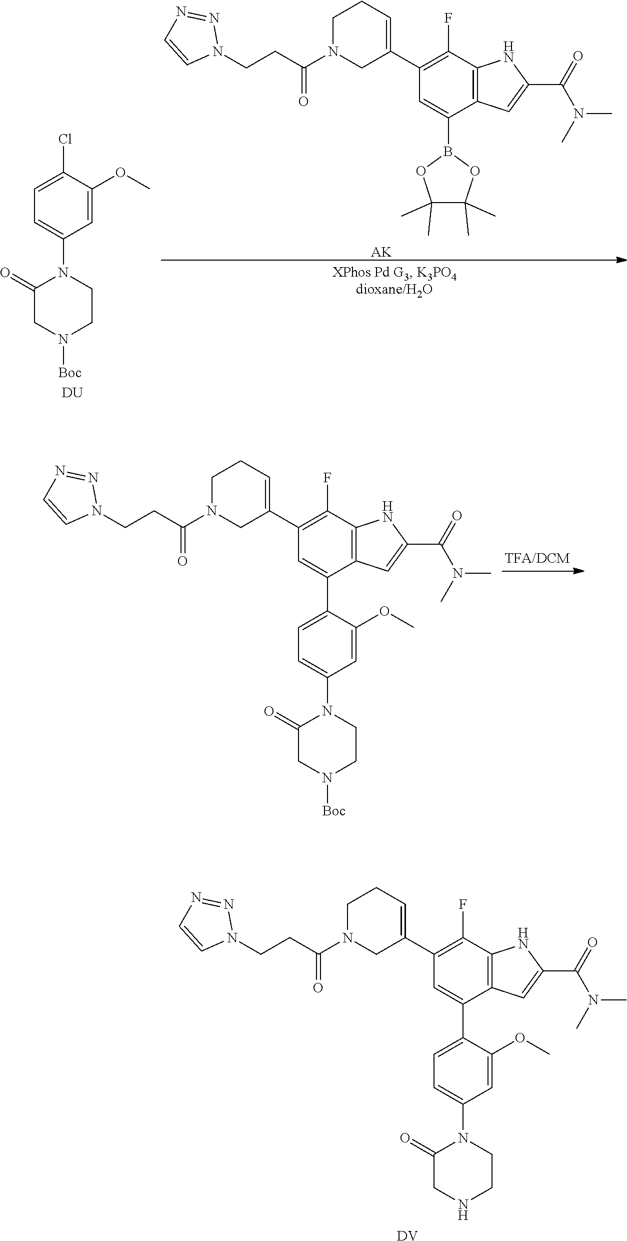

### Step 1—Tert-butyl 4-(4-(6-(1-(3-(1H-1,2,3-triazol-1-yl)propanoyl)-1,2,5,6-tetrahydropyridin-3-yl)-2-(dimethylcarbamoyl)-7-fluoro-1H-indol-4-yl)-3-methoxyphenyl)-3-oxopiperazine-1-carboxylate

A mixture of tert-butyl 4-(4-chloro-3-methoxyphenyl)-3-oxopiperazine-1-carboxylate (127 mg, 373 μmol, Intermediate DU), 6-(1-(3-(1H-1,2,3-triazol-1-yl)propanoyl)-1,2,5,6-tetrahydropyridin-3-yl)-7-fluoro-N,N-dimethyl-4-(4,4,5,5-tetramethyl-1,3,2-dioxaborolan-2-yl)-1H-indole-2-carboxamide (0.20 g, 373 μmol, Intermediate AK), XPhos Pd G3 (31.6 mg, 37.3 μmol) and K₃PO₄ (237 mg, 1.12 mmol) in dioxane (2.0 mL) and H₂O (0.4 mL) was degassed and purged with N₂ three time. Then the mixture was stirred at 90° C. for 1.5 hr under N₂ atmosphere. On completion, the reaction mixture was concentrated under reduced pressure to give a residue. The residue was purified by flash silica gel chromatography (ISCO®; 120 g Sepa Flash® Silica Flash Column, Eluent of 0˜9% Ethyl acetate/Petroleum ether gradient @, 100 mL/min) to give the title compound (211 mg, 75% yield) as a yellow oil. LC-MS (ESI⁺) m/z 715.0 (M+H); ¹H NMR (400 MHz, CDCl₃) δ ppm 9.39 (d, J=8.4 Hz, 1H), 7.76 (d, J=4.4 Hz, 1H), 7.67 (d, J=3.6 Hz, 1H), 7.40 (dd, J=6.0, 7.6 Hz, 1H), 7.02 (s, 1H), 6.98-6.93 (m, 2H), 6.68 (d, J=2.0 Hz, 1H), 6.15 (d, J=17.2 Hz, 1H), 4.80 (q, J=5.6 Hz, 2H), 4.48 (s, 1H), 4.33-4.26 (m, 3H), 3.88-3.76 (m, 8H), 3.58 (t, J=5.6 Hz, 1H), 3.42-3.15 (m, 6H), 3.06 (q, J=6.4 Hz, 2H), 2.36 (s, 2H), 1.53 (s, 9H).

### Step 2—6-(1-(3-(1H-1,2,3-triazol-1-yl)propanoyl)-1,2,5,6-tetrahydropyridin-3-yl)-7-fluoro-4-(2-methoxy-4-(2-oxopiperazin-1-yl)phenyl)-N,N-dimethyl-1H-indole-2-carboxamide

To a solution of tert-butyl 4-(4-(6-(1-(3-(1H-1,2,3-triazol-1-yl)propanoyl)-1,2,5,6-tetrahydropyridin-3-yl)-2-(dimethylcarbamoyl)-7-fluoro-1H-indol-4-yl)-3-methoxyphenyl)-3-oxopiperazine-1-carboxylate (211 mg, 251 mol) in DCM (2.00 mL) was added TFA (522 mg, 4.58 mmol, 340 L). The mixture was then stirred at 25° C. for 0.5 hr. On completion, the reaction mixture was concentrated under reduced pressure to give the title compound (351 mg, TFA) as brown gum. LC-MS (ESI⁺) m/z 615.1 (M+H)⁺.

### Tert-butyl 4-(4-bromo-3-chlorophenyl)piperazine-1-carboxylate (Intermediate DW)

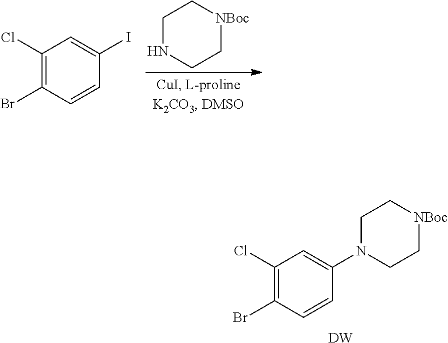

A mixture of 1-bromo-2-chloro-4-iodo-benzene (5 g, 15.7 mmol, CAS #535934-25-9), tert-butyl piperazine-1-carboxylate (5.87 g, 31.5 mmol, CAS #57260-71-6), CuI (600 mg, 3.15 mmol), K₂CO₃ (6.53 g, 47.3 mmol) and L-proline (363 mg, 3.15 mmol) in DMSO (50 mL) was degassed and purged with N₂ three times. Then the mixture was stirred at 80° C. for 20 hrs under N₂ atmosphere. On completion, the reaction mixture was quenched with water (200 mL) and extracted with ethyl acetate (3×100 mL). The combined organic layers were washed with brine (300 mL) and dried over anhydrous sodium sulfate, filtered and the filtrate was concentrated in vacuo to give the crude residue. The residue was purified by column chromatography (SiO₂, Petroleum ether/Ethyl acetate=1/0 to 7/1) to give the title compound (2.7 g, 46% yield) as a yellow solid. LC-MS (ESI⁺) mi/z 321.0 (M-55).

### 6-(1-Acetyl-3,6-dihydro-2H-pyridin-5-yl)-4-(2-chloro-4-piperazin-1-yl-phenyl)-7-fluoro-N,N-dimethyl-LH-indole-2-carboxamide (Intermediate DX)

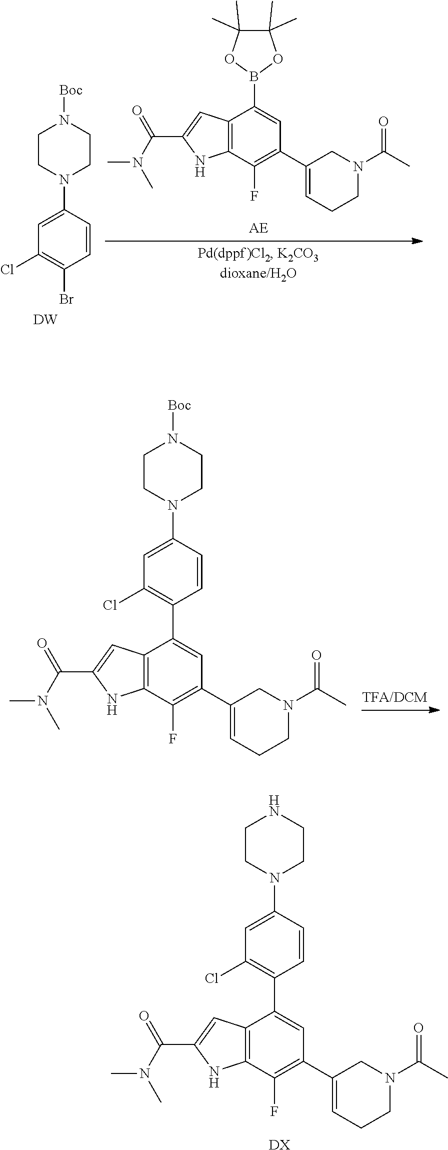

### Step 1—Tert-butyl 4-[4-[6-(1-acetyl-3,6-dihydro-2H-pyridin-5-yl)-2-(dimethylcarbamoyl)-7-fluoro-1H-indol-4-yl]-3-chloro-phenyl]piperazine-1-carboxylate

A mixture of tert-butyl 4-(4-bromo-3-chloro-phenyl)piperazine-1-carboxylate (450 mg, 1.20 mmol, Intermediate DW), 6-(1-acetyl-1,2,5,6-tetrahydropyridin-3-yl)-7-fluoro-N,N-dimethyl-4-(4,4,5,5-tetramethyl-1,3,2-dioxaborolan-2-yl)-1H-indole-2-carboxamide (545 mg, 1.20 mmol, Intermediate AE), Pd(dppf)Cl₂ (87.0 mg, 119 μmol) and K₂CO₃ (496 mg, 3.59 mmol) in dioxane (5.0 mL) and H₂O (0.5 mL) was degassed and purged with N₂ three times. Then the mixture was stirred at 85° C. for 1 hr under N₂ atmosphere. On completion, the mixture was diluted with water (50 mL) and extracted with ethyl acetate (50 mL×2), dried over sodium sulfate, filtered and concentrated under reduced pressure. The residue was purified by silica gel column chromatography (petroleum ether/ethyl acetate=5/1˜1/1) to give the title compound (470 mg, 56% yield) as a white solid. LC-MS (ESI⁺) m/z 624.2 (M+H)⁺.

### Step 2—6-(1-Acetyl-3,6-dihydro-2H-pyridin-5-yl)-4-(2-chloro-4-piperazin-1-yl-phenyl)-7-fluoro-N,N-dimethyl-1H-indole-2-carboxamide

To a solution of tert-butyl 4-[4-[6-(1-acetyl-3,6-dihydro-2H-pyridin-5-yl)-2-(dimethylcarbamoyl)-7-fluoro-1H-indol-4-yl]-3-chloro-phenyl]piperazine-1-carboxylate (480 mg, 769 μmol) in DCM (5.0 mL) was added TFA (1.84 g, 16.1 mmol, 1.2 mL). The mixture was then stirred at 20° C. for 0.5 h. On completion, the mixture was concentrated under reduced pressure using a rotary evaporator to give the title compound (1 g, 7TFA) as a white solid. LC-MS (ESI⁺) m/z 524.1 (M+H)⁺.

### 6-(1-(3-(1H-pyrazol-1-yl)propanoyl)-1,2,5,6-tetrahydropyridin-3-yl)-7-fluoro-N,N-dimethyl-4-(4,4,5,5-tetramethyl-1,3,2-dioxaborolan-2-yl)benzo[b]thiophene-2-carboxamide (Intermediate DY)

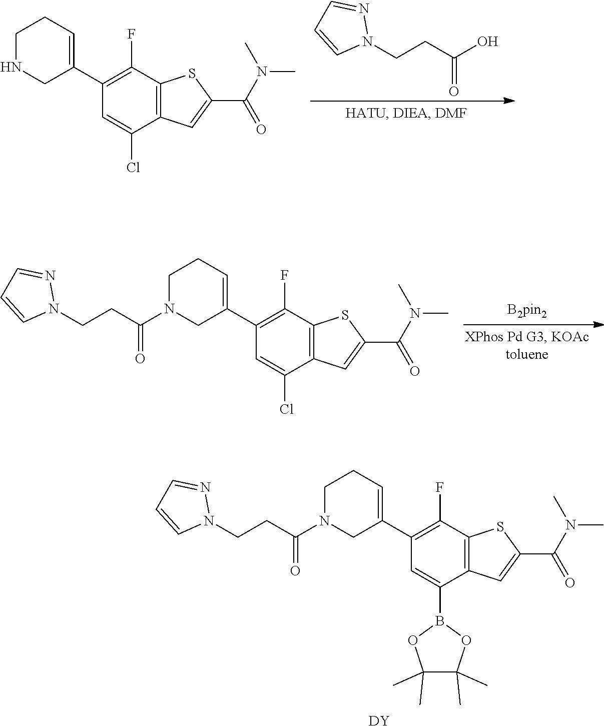

### Step 1—6-(1-(3-(1H-pyrazol-1-yl)propanoyl)-1,2,5,6-tetrahydropyridin-3-yl)-4-chloro-7-fluoro-N,N-dimethylbenzo[b]thiophene-2-carboxamide

To a solution of 3-pyrazol-1-ylpropanoic acid (695 mg, 4.96 mmol, CAS #89532-73-0) in DMF (28 mL) was added DIEA (3.20 g, 24.8 mmol, 4.32 mL) and HATU (3.53 g, 9.30 mmol) at 0° C. and the mixture was stirred for 0.1 hr. Then added 4-chloro-7-fluoro-N,N-dimethyl-6-(1,2,3,6-tetrahydropyridin-5-yl)benzothiophene-2-carboxamide (2.10 g, 6.20 mmol, synthesized via Steps 1-6 of Intermediate BY) was added and the mixture was stirred at 0° C. for 0.9 hr. On completion, the reaction mixture was diluted with H₂O (60 mL) and extracted with EA (30 mL×3). The combined organic layers were washed with brine (20 mL×3), dried over Na₂SO₄, filtered and concentrated under reduced pressure to give a residue. The residue was purified by column chromatography (SiO₂, Petroleum ether/Ethyl acetate=4/1 to 0/1) to give the title compound (880 mg, 26% yield) as a white solid. LC-MS (ESI⁺) m/z 461.0 (M+H)⁺.

### Step 2—6-(1-(3-(1H-pyrazol-1-yl)propanoyl)-1,2,5,6-tetrahydropyridin-3-yl)-7-fluoro-N,N-dimethyl-4-(4,4,5,5-tetramethyl-1,3,2-dioxaborolan-2-yl)benzo[b]thiophene-2-carboxamide

A mixture of 4-chloro-7-fluoro-N,N-dimethyl-6-[1-(3-pyrazol-1-ylpropanoyl)-3,6-dihydro-2H-pyridin-5-yl]benzothiophene-2-carboxamide (700 mg, 1.52 mmol), KOAc (447 mg, 4.56 mmol), XPhos Pd G3 (129 mg, 152 mol) and 4,4,5,5-tetramethyl-2-(4,4,5,5-tetramethyl-1,3,2-dioxaborolan-2-yl)-1,3,2-dioxaborolane (771 mg, 3.04 mmol) in dioxane (10 mL) was degassed and purged with N₂ three times. Then the mixture was stirred at 80° C. for 1 hr under N₂ atmosphere. On completion, the reaction mixture was diluted with H₂O (60 mL) and extracted with EA (30 mL×3). The combined organic layers were washed with aqueous NaCl (30 mL×2), dried over Na₂SO₄, filtered and concentrated under reduced pressure to give a residue. The residue was purified by column chromatography (SiO₂, Petroleum ether/Ethyl acetate=4/1 to 1/1) to give the title compound (500 mg, 50% yield) as a white solid. LC-MS (ESI⁺) m/z 553.3 (M+H)⁺.

### 1-(4-Chloro-3-(trifluoromethyl)phenyl)piperazine (Intermediate DZ)

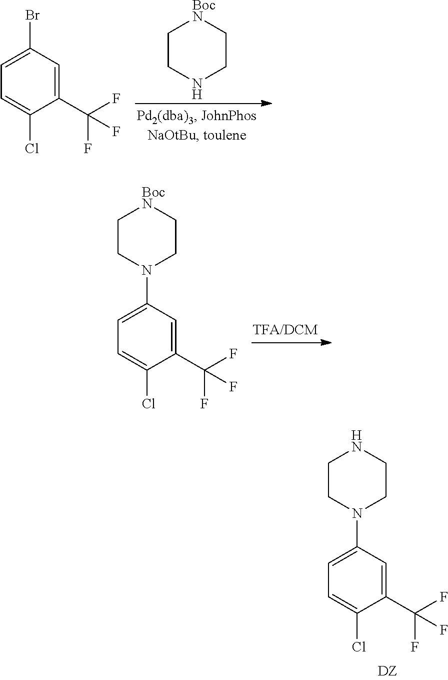 [SMILES: FC(F)(F)c1cc(Br)ccc1Cl ; CC(C)(C)OC(=O)N1CCNCC1 ; CC(C)(C)OC(=O)N1CCN(c2ccc(Cl)c(C(F)(F)F)c2)CC1 ; FC(F)(F)c1cc(N2CCNCC2)ccc1Cl]

### Step 1—Tert-butyl 4-(4-chloro-3-(trifluoromethyl)phenyl)piperazine-1-carboxylate

A mixture of 4-bromo-1-chloro-2-(trifluoromethyl)benzene (2 g, 7.71 mmol, 1.15 mL), tert-butyl piperazine-1-carboxylate (2.15 g, 11.6 mmol), Pd₂(dba)₃ (706 mg, 770 μmol), JohnPhos (230 mg, 770 μmo) and NaOBu-t (2 M, 11.56 mL) in anhydrous toluene (20 mL) was degassed and purged with nitrogen three times. Then the reaction was stirred at 60° C. for 8 hrs under nitrogen atmosphere. On completion, the reaction mixture was quenched with water (20 mL) and extracted with ethyl acetate (3×20 mL). The combined organic layers were washed with brine (10 mL) and dried over anhydrous sodium sulfate, filtered and the filtrate was concentrated in vacuo to give the crude residue. The crude residue was purified by reverse phase HPLC (0.1% FA condition) to give the title compound (1.8 g, 58% yield) as a white solid. LC-MS (ESI⁺) m/z 308.9 (M-55)⁺.

### Step 2—1-(4-Chloro-3-(trifluoromethyl)phenyl)piperazine

To a solution of tert-butyl 4-(4-chloro-3-(trifluoromethyl)phenyl)piperazine-1-carboxylate (1.5 g, 4.11 mmol) in DCM (15 mL) was added TFA (3 mL, 40.4 mmol). The mixture was then stirred at 25° C. for 1 hr. On completion, the reaction mixture was filtered and the filtrate was concentrated in vacuo to give the crude residue. The crude residue was purified by reverse phase HPLC (0.1% NH₃H₂O) to give the title compound (600 mg, 54% yield) as a white solid. LC-MS (ESI⁺) m/z 264.9 (M+H)⁺.

### 3-(4-(2-(4-(4-Chloro-3-(trifluoromethyl)phenyl)piperazin-1-yl)ethyl)-3-methyl-2-oxo-2,3-dihydro-1H-benzo[d]imidazol-1-yl)piperidine-2,6-dione (Intermediate EA)

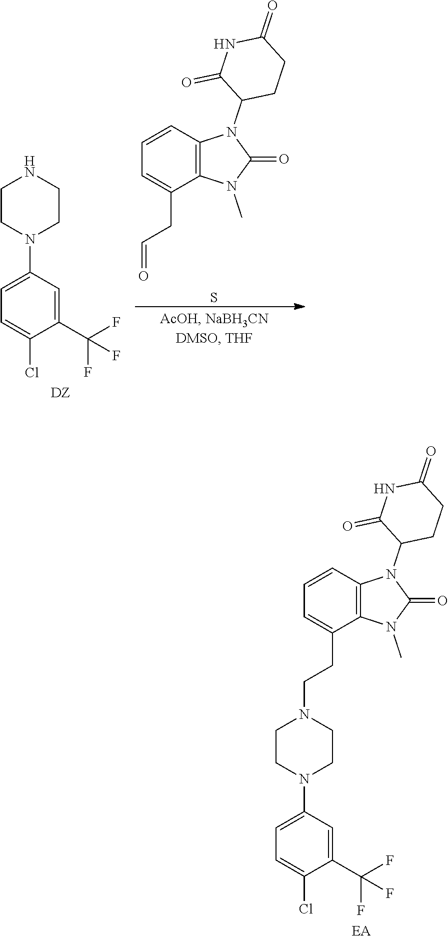

To a solution of 1-(4-chloro-3-(trifluoromethyl)phenyl)piperazine (200 mg, 756 μmol, Intermediate DZ) and 2-(1-(2,6-dioxopiperidin-3-yl)-3-methyl-2-oxo-2,3-dihydro-1H-benzo[d]imidazol-4-yl)acetaldehyde (228 mg, 756 μmol, Intermediate S) in DMSO (2 mL) and THE (2 mL) was added AcOH (90.8 mg, 1.51 mmol) and stirred for 1 hr. Then, NaBH(OAc)₃ (480 mg, 2.27 mmol) was added at 0° C. Then the mixture was stirred at 0-25° C. for 2 hrs. On completion, the reaction mixture was filtered and the filtrate was concentrated in vacuo to give the crude residue. The crude residue was purified by reverse phase HPLC (0.1% FA condition) to give the title compound (100 mg, 19% yield) as a yellow solid. LC-MS (ESI) m/z 550.1 (M+H)⁺.

### 4-(4-(4-Chloro-3-methoxyphenyl)piperidin-1-yl)-3-fluoroaniline (Intermediate EB)

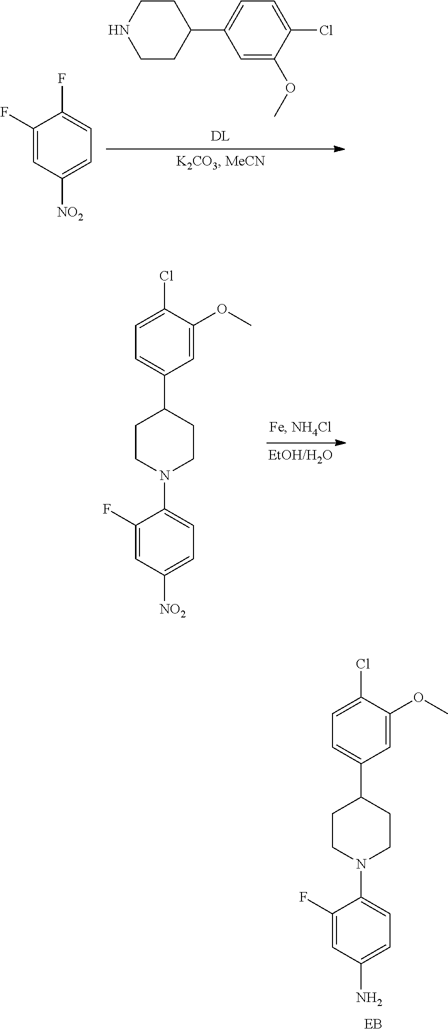 [SMILES: O=[N+]([O-])c1ccc(F)c(F)c1 ; COc1cc(C2CCNCC2)ccc1Cl ; COc1cc(C2CCN(c3ccc([N+](=O)[O-])cc3F)CC2)ccc1Cl ; COc1cc(C2CCN(c3ccc(N)cc3F)CC2)ccc1Cl]

### Step 1—4-(4-Chloro-3-methoxyphenyl)-1-(2-fluoro-4-nitrophenyl)piperidine

To a solution of 1,2-difluoro-4-nitro-benzene (1.00 g, 6.29 mmol. CAS #369-34-6) and 4-(4-chloro-3-methoxy-phenyl)piperidine (2.14 g, 8.17 mmol, Intermediate DL) in MeCN (20 mL) was added K₂CO₃ (2.17 g, 15.7 mmol). The mixture was then stirred at 80° C. for 2 hrs. On completion, the mixture was diluted with water (10 mL) and extracted with ethyl acetate (20 mL×2), dried over sodium sulfate, filtered and concentrated under reduced pressure. The residue was purified by silica gel column chromatography (petroleum ether/ethyl acetate=5/1) to give the title compound (1.3 g, 52% yield) as a white solid. LC-MS (ESI⁺) m/z 364.7 (M+H)⁺.

### Step 2—4-(4-(4-Chloro-3-methoxyphenyl)piperidin-1-yl)-3-fluoroaniline

To a solution of 4-(4-chloro-3-methoxy-phenyl)-1-(2-fluoro-4-nitro-phenyl)piperidine (0.50 g, 1.37 mmol) in EtOH (5 mL) and H₂O (5 mL) was added Fe (383 mg, 6.85 mmol) and NH₄Cl (587 mg, 11.0 mmol). The mixture was then stirred at 50° C. for 12 hrs. On completion, the reaction mixture was filtered and the residue was extracted with DCM 30 mL (10 mL×3). The combined organic layers were washed with brine (10 mL), dried over Na₂SO₄, filtered and concentrated under reduced pressure to give a residue. The residue was purified by silica gel column chromatography (petroleum ether/ethyl acetate=5/1) to give the title compound (0.35 g, 69% yield) as a yellow solid. LC-MS (ESI⁺) m/z 334.7 (M+H)⁺.

### 3-((4-(4-(4-Chloro-3-methoxyphenyl)piperidin-1-yl)-3-fluorophenyl)amino)piperidine-2,6-dione (Intermediate EC)

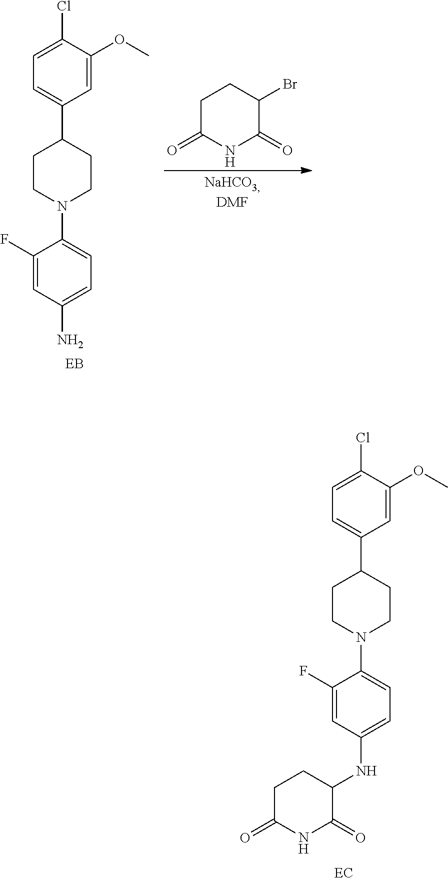

To a solution of 4-[4-(4-chloro-3-methoxy-phenyl)-1-piperidyl]-3-fluoro-aniline (300 mg, 896 mol, Intermediate EB) and 3-bromopiperidine-2,6-dione (516 mg, 2.69 mmol, CAS #62595-74-8) in DMF (5 mL) was added NaHCO₃ (376 mg, 4.48 mmol). The mixture was then stirred at 80° C. for 12 hrs. On completion, the reaction mixture was quenched by addition of NH₄Cl (2 mL), and then extracted with EtOAc (10 mL×3). The combined organic layers were washed with brine (10 mL×3), dried over Na₂SO₄, filtered and concentrated under reduced pressure to give a residue. The residue was purified by prep-HPLC (0.8 g/L NH₄HCO₃ condition) to give the title compound (200 mg, 48% yield) as a black solid. LC-MS (ESI⁺) m/z 445.8 (M+H)⁺.

### Tert-butyl 4-(4-bromo-5-chloro-2-fluorophenyl)piperazine-1-carboxylate (Intermediate ED)

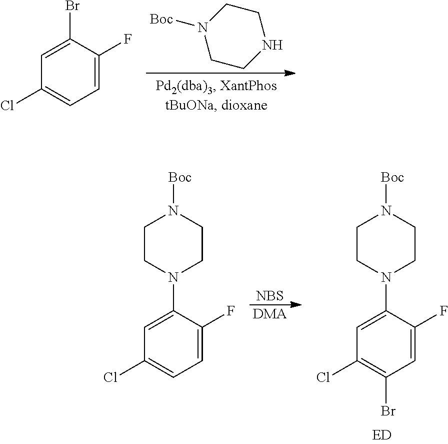

### Step 1—Tert-butyl 4-(5-chloro-2-fluorophenyl)piperazine-1-carboxylate

A mixture of tert-butyl piperazine-1-carboxylate (4.9 g, 26.3 mmol, CAS #57260-71-6), 2-bromo-4-chloro-1-fluoro-benzene (5.00 g, 23.9 mmol, CAS #1996-30-1), Pd₂(dba)₃ (109 mg, 119 mol), Xantphos (152 mg, 263 μmol) and tBuONa (688 mg, 7.16 mmol) in dioxane (50 mL) was degassed and purged with N₂ three times. Then the mixture was stirred at 100° C. for 1 hr under N₂ atmosphere. On completion, the mixture was concentrated under reduced pressure. The residue was purified by silica gel column chromatography (petroleum ether/ethyl acetate=10/1) to give the title compound (5.2 g) as a yellow oil. LC-MS (ESI⁺) m/z 215.1 (M-99)⁺.

### Step 2—Tert-butyl 4-(4-bromo-5-chloro-2-fluorophenyl)piperazine-1-carboxylate

To a solution of tert-butyl 4-(5-chloro-2-fluoro-phenyl)piperazine-1-carboxylate (2.50 g, 7.94 mmol) in DMA (20 mL) was added NBS (1.27 g, 7.15 mmol). The mixture was then stirred at −10° C. for 5 min. On completion, the mixture was diluted with water (3 mL). The residue was purified by prep-HPLC (0.1% FA condition) to give the title compound (1.2 g, 35% yield) as a white solid. LC-MS (ESI⁺) m/z 392.9 (M+H)⁺.

### 6-(1-Acetyl-1,2,5,6-tetrahydropyridin-3-yl)-4-(2-chloro-5-fluoro-4-(piperazin-1-yl)phenyl)-7-fluoro-N,N-dimethyl-1H-indole-2-carboxamide (Intermediate EE)

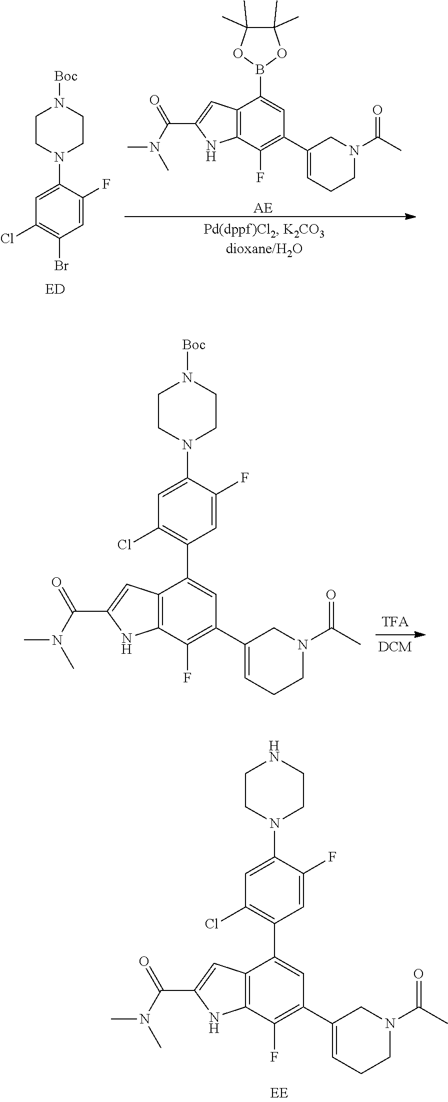

### Step 1—Tert-butyl 4-(4-(6-(1-acetyl-1,2,5,6-tetrahydropyridin-3-yl)-2-(dimethylcarbamoyl)-7-fluoro-1H-indol-4-yl)-5-chloro-2-fluorophenyl)piperazine-1-carboxylate

A mixture of tert-butyl 4-(4-bromo-5-chloro-2-fluoro-phenyl)piperazine-1-carboxylate (500 mg, 1.27 mmol, Intermediate ED), 6-(1-acetyl-3,6-dihydro-2H-pyridin-5-yl)-7-fluoro-N,N-dimethyl-4-(4,4,5,5-tetramethyl-1,3,2-dioxaborolan-2-yl)-1H-indole-2-carboxamide (578 mg, 1.27 mmol, Intermediate AE), Pd(dppf)Cl₂·CH₂Cl₂ (104 mg, 127 μmol), and K₂CO₃ (527 mg, 3.81 mmol) in dioxane (6 mL) and H₂O (1 mL) was degassed and purged with N₂ three times. Then the mixture was stirred at 80° C. for 2 hrs under N₂ atmosphere. On completion, the mixture was filtered and concentrated under reduced pressure, and purified by prep-HPLC(0.1% FA condition) to give the title compound (733 mg, 82% yield, FA) as a white solid. LC-MS (ESI⁺) m/z 587.0 (M-55)⁺.

### Step 2—6-(1-Acetyl-1,2,5,6-tetrahydropyridin-3-yl)-4-(2-chloro-5-fluoro-4-(piperazin-1-yl)phenyl)-7-fluoro-N,N-dimethyl-1H-indole-2-carboxamide

To a solution of tert-butyl 4-[4-[6-(1-acetyl-3,6-dihydro-2H-pyridin-5-yl)-2-(dimethylcarbamoyl)-7-fluoro-1H-indol-4-yl]-5-chloro-2-fluoro-phenyl]piperazine-1-carboxylate (733 mg, 1.14 mmol) in DCM (6 mL) was added TFA (2.30 g, 20.2 mmol). The mixture was then stirred at 25° C. for 1 hr. On completion, N₂ was blown over the reaction mixture to remove solvent and give the title compound (600 mg) as a yellow solid. LC-MS (ESI⁺) m/z 542.1 (M+H)⁺.

### 4-(4-(4-Chloro-3-(trifluoromethoxy)phenyl)piperidin-1-yl)-5-fluoro-2-methoxyaniline (Intermediate EF)

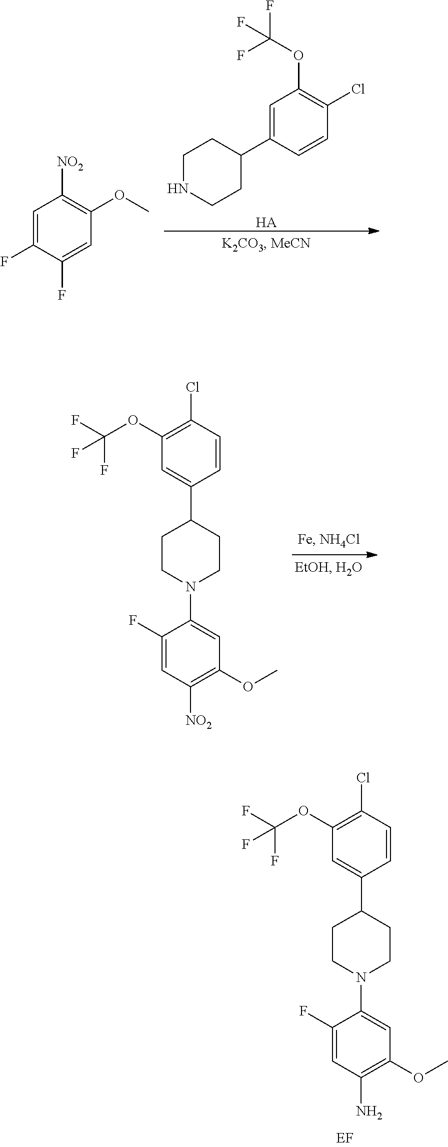

### Step 1—4-(4-Chloro-3-(trifluoromethoxy)phenyl)-1-(2-fluoro-5-methoxy-4-nitrophenyl)piperidine

To a solution of 1,2-difluoro-4-methoxy-5-nitro-benzene (800 mg, 4.2 mmol, synthesized via Step 1 of Intermediate AQ) in MeCN (40 mL) was added K₂CO₃ (2.9 g, 21.1 mmol) and 4-[4-chloro-3-(trifluoromethoxy)phenyl]piperidine (1.6 g, 5 mmol, HCl. Intermediate HA). The mixture was then stirred at 80° C. for 12 hrs. On completion, the residue was diluted with water (100 mL) and extracted with EA (100 mL×2). The combined organic layers were washed with brine (50 mL×2), dried over Na₂SO₄, filtered and concentrated under reduced pressure to give a residue. The residue was purified by column chromatography (SiO₂, Petroleum ether/Ethyl acetate=30/1 to 10/1) to give the title compound (1.9 g, 97% yield) as a yellow solid. LC-MS (ESI⁺) m/z 449.0 (M+H)⁺.

### Step 2—4-(4-(4-Chloro-3-(trifluoromethoxy)phenyl)piperidin-1-yl)-5-fluoro-2-methoxyaniline

To a solution of 4-[4-chloro-3-(trifluoromethoxy)phenyl]-1-(2-fluoro-5-methoxy-4-nitro-phenyl)piperidine (1.9 g, 4.2 mmol) in EtOH (10 mL) and H₂O (10 mL) was added Fe (1.1 g, 21.1 mmol) and NH₄C1 (1.8 g, 33.8 mmol). The mixture was then stirred at 25° C. for 12 hrs. On completion, the combined organic layers were washed with EA (100 mL×2), dried over Na₂SO₄, filtered and the filtrate was concentrated under reduced pressure to give a residue. On completion, the reaction mixture was concentrated under reduced pressure to give the title compound (1.74 g) as a yellow oil. LC-MS (ESI⁺) m/z 419.1 (M+H)⁺.

### 3-((4-(4-(4-Chloro-3-(trifluoromethoxy)phenyl)piperidin-1-yl)-5-fluoro-2-methoxyphenyl)amino)piperidine-2,6-dione (Intermediate EG)

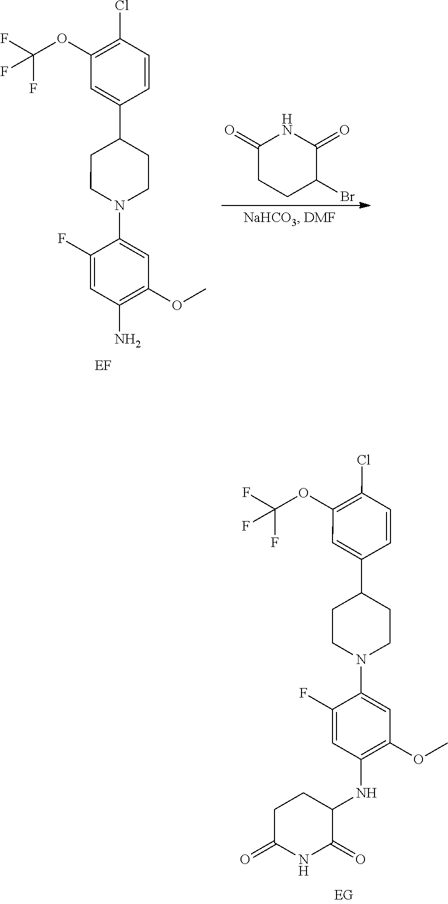 [SMILES: COc1cc(N2CCC(c3ccc(Cl)c(OC(F)(F)F)c3)CC2)c(F)cc1N ; O=C1CCC(Br)C(=O)N1 ; COc1cc(N2CCC(c3ccc(Cl)c(OC(F)(F)F)c3)CC2)c(F)cc1NC1CCC(=O)NC1=O]

To a solution of 4-[4-[4-chloro-3-(trifluoromethoxy)phenyl]-1-piperidyl]-5-fluoro-2-methoxy-aniline (1.7 g, 4.1 mmol, Intermediate EF) in DMF (20 mL) was added NaHCO₃ (698 mg, 8.3 mmol, 323.3 μL) and 3-bromopiperidine-2,6-dione (1.60 g, 8.31 mmol). The mixture was then stirred at 80° C. for 12 hr. On completion, the reaction mixture was concentrated under reduced pressure to remove DMF. The residue was purified by prep-HPLC (0.1% FA) to give the title compound (950 mg, 39% yield, FA) as a green solid. LC-MS (ESI⁺) m/z 529.9 (M+H)⁺.

### Tert-butyl 4-(4-(6-chloro-2-(dimethylcarbamoyl)-7-fluorobenzo[b]thiophen-4-yl)phenyl)piperazine-1-carboxylate (Intermediate EH)

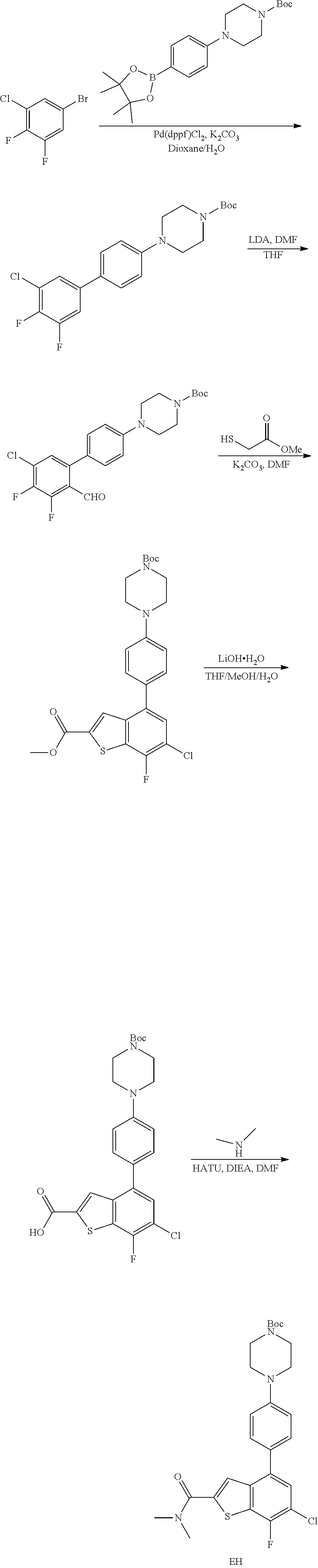

### Step 1—Tert-butyl 4-(3′-chloro-4′,5′-difluoro-[1,1′-biphenyl]-4-yl)piperazine-1-carboxylate

A mixture of 5-bromo-1-chloro-2,3-difluoro-benzene (12.0 g, 52.8 mmol, CAS #1060813-07-1), tert-butyl 4-[4-(4,4,5,5-tetramethyl-1,3,2-dioxaborolan-2-yl)phenyl]piperazine-1-carboxylate (20.5 g, 52.8 mmol, CAS #470478-90-1), Pd(dppf)Cl₂·CH₂Cl₂ (4.31 g, 5.28 mmol), and K₂CO₃ (21.9 g, 158 mmol) in dioxane (40 mL) and H₂O (10 mL) was degassed and purged with N₂ three times. Then the mixture was stirred at 80° C. for 2 hrs under N₂ atmosphere. On completion, the mixture was diluted with water (10 mL) and extracted with ethyl acetate (40 mL×3), dried over sodium sulfate, filtered and concentrated under reduced pressure. The residue was purified by silica gel column chromatography (petroleum ether/ethyl acetate=1/0˜10/1) to give the title compound (20 g, 86% yield) as a yellow solid. LC-MS (ESI⁺) m/z 409.2 (M+H)⁺.

### Step 2—Tert-butyl 4-(5′-chloro-3′,4′-difluoro-2′-formyl-[1,1′-biphenyl]-4-yl)piperazine-1-carboxylate

A mixture of tert-butyl 4-[4-(3-chloro-4,5-difluoro-phenyl)phenyl]piperazine-1-carboxylate (7.00 g, 17.1 mmol) in THF (50 mL) was degassed and purged with N₂ three times. Then LDA (2 M, 10.3 mL) was added, and the mixture was stirred at −78° C. for 2 hrs under N₂ atmosphere. Next, DMF (2.50 g, 2.63 mL) was added and the mixture was stirred at −78° C. for 2 hrs under N₂ atmosphere. On completion, the mixture was diluted with NH₄Cl (20 mL) and extracted with dichloromethane (50 mL×3), dried over sodium sulfate, filtered and concentrated under reduced pressure. The residue was purified by silica gel column chromatography (petroleum ether/ethyl acetate=10/1) to give the title compound (7.1 g, 87% yield) as a yellow solid. LC-MS (ESI⁺) m/z 437.0 (M+H)⁺.

### Step 3—Tert-butyl 4-(4-(6-chloro-7-fluoro-2-(methoxycarbonyl)benzo[b]thiophen-4-yl)phenyl)piperazine-1-carboxylate

To a solution of tert-butyl 4-[4-(5-chloro-3,4-difluoro-2-formyl-phenyl)phenyl]piperazine-1-carboxylate (6.00 g, 13.7 mmol) in DMF (130 mL) was added methyl 2-sulfanylacetate (1.75 g, 16.5 mmol, CAS #2365-48-2) and K₂CO₃ (3.80 g, 27.5 mmol). The mixture was stirred at 20° C. for 1 hr. On completion, the mixture was diluted with water (200 mL) and filtered to give the filter cake as the title compound (7.5 g) as a yellow solid. LC-MS (ESI⁺) m/z 505.0 (M+H)⁺.

### Step 4—4-(4-(4-(Tert-butoxycarbonyl)piperazin-1-yl)phenyl)-6-chloro-7-fluorobenzo[b]thiophene-2-carboxylic acid

To a solution of tert-butyl 4-(4-(6-chloro-7-fluoro-2-(methoxycarbonyl)benzo[b]thiophen-4-yl)phenyl)piperazine-1-carboxylate (1 g, 1.98 mmol) in THF (20 mL), MeOH (5 mL) and H₂O (5 mL) was added LiOH·H₂O (207 mg, 4.95 mmol). The mixture was then stirred at 25° C. for 2 h. On completion, the reaction mixture was diluted with H₂O (30 mL) and extracted with EA (3×100 mL). The combined organic layers were dried over Na₂SO₄, filtered and concentrated under reduced pressure to give the title compound (900 mg) as a white solid. LC-MS (ESI⁺) m/z 491.0 (M+H)⁺.

### Step 5—Tert-butyl 4-(4-(6-chloro-2-(dimethylcarbamoyl)-7-fluorobenzo[b]thiophen-4-yl)phenyl)piperazine-1-carboxylate

To a solution of 4-(4-(4-(tert-butoxycarbonyl)piperazin-1-yl)phenyl)-6-chloro-7-fluorobenzo[b]thiophene-2-carboxylic acid (1 g, 2.04 mmol) in DMF (10 mL) was added HATU (1.16 g, 3.06 mmol), DIEA (1.32 g, 10.1 mmol, 1.77 mL) and N-methylmethanamine (137 mg, 1.69 mmol, 154 KL, HCl). The mixture was then stirred at 25° C. for 2 hr. On completion, the reaction mixture was triturated with H₂O (100 mL) for 15 min, then filtered and the filter cake was dried under reduced pressure to give the title compound (500 mg) as a white solid. LC-MS (ESI⁺) m/z 462.0 (M-55)⁺.

### 6-(1-(3-(1H-pyrazol-1-yl)propanoyl)-1,2,5,6-tetrahydropyridin-3-yl)-7-fluoro-N,N-dimethyl-4-(4-(piperazin-1-yl)phenyl)benzo[b]thiophene-2-carboxamide (Intermediate EI)

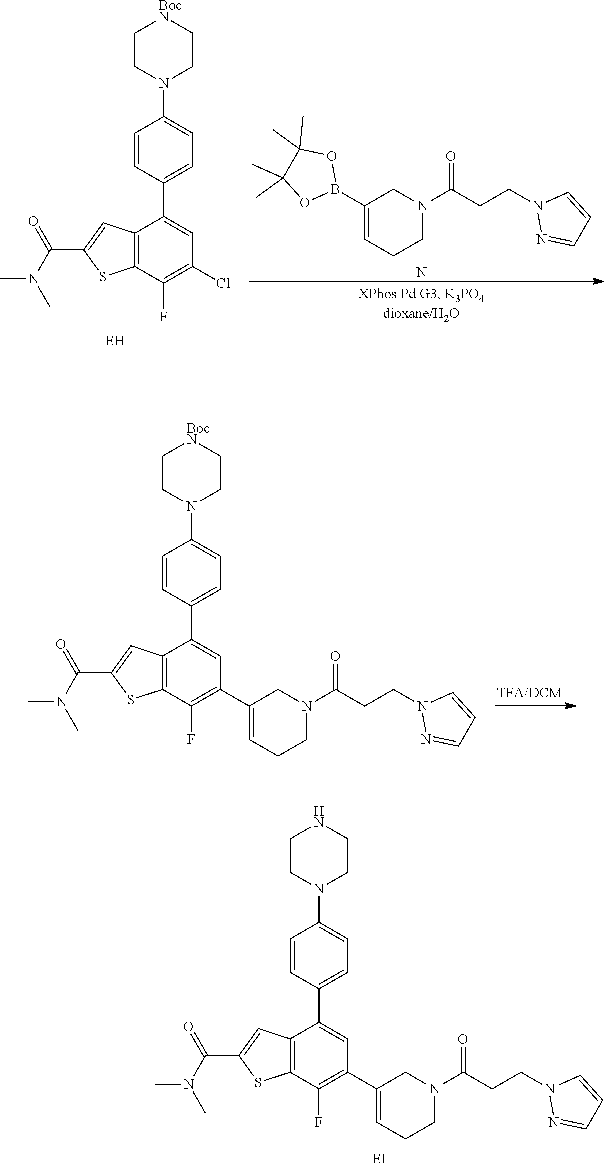

### Step 1—Tert-butyl 4-(4-(6-(1-(3-(1H-pyrazol-1-yl)propanoyl)-1,2,5,6-tetrahydropyridin-3-yl)-2-(dimethylcarbamoyl)-7-fluorobenzo[b]thiophen-4-yl)phenyl)piperazine-1-carboxylate

To a solution of tert-butyl 4-(4-(6-chloro-2-(dimethylcarbamoyl)-7-fluorobenzo[b]thiophen-4-yl)phenyl)piperazine-1-carboxylate (450 mg, 868 μmol, Intermediate EH) in dioxane (2 mL) and H₂O (0.5 mL) was added 3-(1H-pyrazol-1-yl)-1-(5-(4,4,5,5-tetramethyl-1,3,2-dioxaborolan-2-yl)-3,6-dihydropyridin-1(2H)-yl)propan-1-one (261 mg, 789 μmol, Intermediate N), K₃PO₄ (502 mg, 2.37 mmol) and XPhos Pd G3 (66.8 mg, 78.9 mol). The reaction was then stirred at 80° C. for 2 hrs. On completion, the reaction mixture was concentrated in vacuo to give the title compound (500 mg) as a brown solid. LC-MS (ESI⁺) m/z 687.2. (M+H)⁺.

### Step 2—6-(1-(3-(1H-pyrazol-1-yl)propanoyl)-1,2,5,6-tetrahydropyridin-3-yl)-7-fluoro-N,N-dimethyl-4-(4-(piperazin-1-yl)phenyl)benzo[b]thiophene-2-carboxamide

To a solution of tert-butyl 4-(4-(6-(1-(3-(1H-pyrazol-1-yl)propanoyl)-1,2,5,6-tetrahydropyridin-3-yl)-2-(dimethylcarbamoyl)-7-fluorobenzo[b]thiophen-4-yl)phenyl)piperazine-1-carboxylate (500 mg, 727 mol) in DCM (5 mL) was added TFA (1.54 g, 13.4 mmol, 1 mL). The reaction was then stirred at 25° C. for 2 hrs. On completion, the reaction mixture was concentrated in vacuo to give the title compound (250 mg) as a brown gum. LC-MS (ESI⁺) m/z 587.2 (M+H)⁺.

### 3-((4-(1-(4-Chlorophenyl)piperidin-4-yl)-5-fluoro-2-methoxyphenyl)amino)piperidine-2,6-dione (Intermediate EJ)

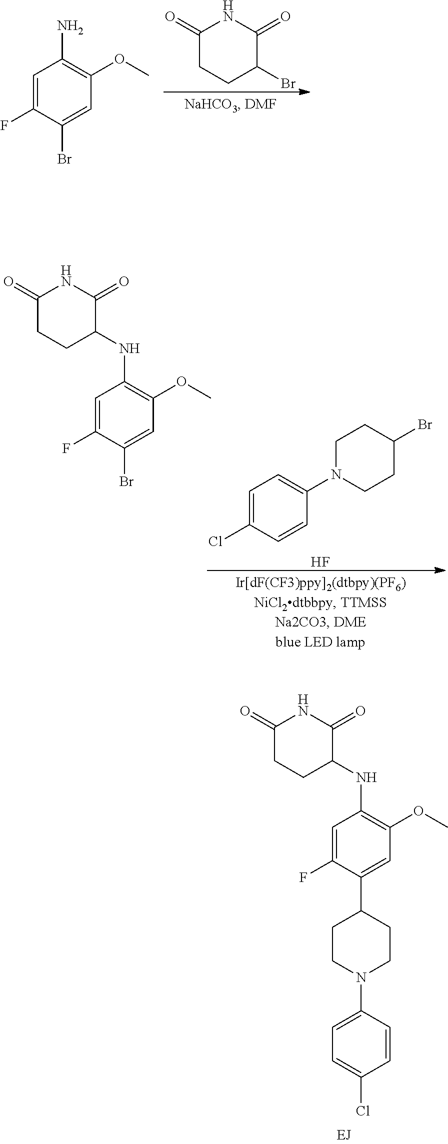

### Step 1—3-((4-Bromo-5-fluoro-2-methoxyphenyl)amino)piperidine-2,6-dione

To a solution of 4-bromo-5-fluoro-2-methoxyaniline (10 g, 50 mmol, CAS #330794-03-1) and 3-bromopiperidine-2,6-dione (21.8 g, 113 mmol, CAS #62595-74-8) in DMF (150 mL) was added NaHCO₃ (9.54 g, 113 mmol, 4.42 mL). Then the mixture was stirred at 80° C. for 12 hr. On completion, the reaction mixture was filtered and quenched by addition of H₂O (20 mL), and then diluted with EA (50 mL) and extracted with EA (50 mL×3). The combined organic layers were washed with brine (50 mL×3), dried over Na₂SO₄, filtered and concentrated under reduced pressure to give a residue. The residue was purified by column chromatography (SiO₂, Petroleum ether/Ethyl acetate=10/1 to 1/1) to give the title compound (2.4 g, 16% yield) as a white solid. LC-MS (ESI⁺) m/z 330.8 (M+H)⁺.

### Step 2—3-((4-(1-(4-Chlorophenyl)piperidin-4-yl)-5-fluoro-2-methoxyphenyl)amino)piperidine-2,6-dione

To a solution of 3-(4-bromo-5-fluoro-2-methoxy-anilino)piperidine-2,6-dione (820 mg, 2.48 mmol), 4-bromo-1-(4-chlorophenyl)piperidine (680 mg, 2.48 mmol, Intermediate HF) in DME (8 mL) was added bis(trimethylsilyl)silyl-trimethyl-silane (677 mg, 2.72 mmol, 840 L), 4-tert-butyl-2-(4-tert-butyl-2-pyridyl)pyridine dichloronickel (29.6 mg, 74.3 μmol), bis[3,5-difluoro-2-[5-(trifluoromethyl)-2-pyridyl]phenyl]iridium(1+) 4-tert-butyl-2-(4-tert-butyl-2-pyridyl)pyridine hexafluorophosphate (55.6 mg, 49.5 μmol) and Na₂CO₃ (411 mg, 4.95 mmol). The mixture was then stirred at 25° C. for 12 hrs. On completion, the reaction mixture was quenched by addition of H₂O (10 mL), and then diluted with EA (10 mL) and extracted with EA (10 mL×2). The combined organic layers were washed with NaCl (10 mL), dried over Na₂SO₄, filtered and concentrated under reduced pressure to give a residue. The crude product was purified by reverse phase HPLC(0.1% NH₄HCO₃) to give the title compound (100 mg, 9% yield) as a white solid. LC-MS (ESI⁺) m/z 445.9 (M+H)⁺.

### Tert-butyl 4-[4-chloro-3-(1,1-difluoroethyl)phenyl]piperazine-1-carboxylate (Intermediate EK)

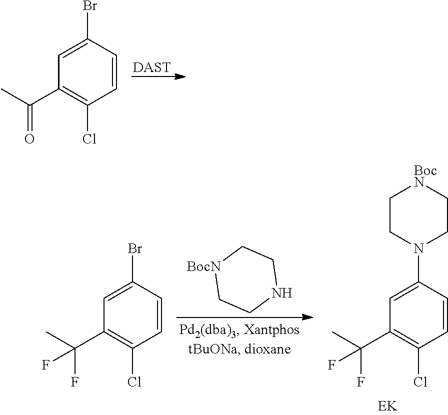

### Step 1—4-Bromo-1-chloro-2-(1,1-difluoroethyl)benzene

A mixture of 1-(5-bromo-2-chloro-phenyl)ethanone (5 g, 21.4 mmol, CAS #105884-19-3) and DAST (61.0 g, 378 mmol, 50.0 mL) was degassed and purged with N₂ three times at 0° C. Then the mixture was stirred at 40° C. for 12 hrs under N₂ atmosphere. On completion, the reaction mixture was quenched by saturated sodium bicarbonate aqueous solution (500 mL) and extracted with ethyl acetate (600×2 mL). The combined organic layers were washed with brine (500 mL) and dried over anhydrous sodium sulfate, filtered and the filtrate was concentrated in vacuo to give the crude residue. The residue was purified by column chromatography (SiO₂, Petroleum ether:Ethyl acetate=1/0 to 20/1) to give the title compound (3.2 g, 59% yield) as colorless oil. ¹H NMR (400 MHz, CDCl₃-d) δ=1.98 (t, J=18.0 Hz, 3H), 7.27 (s, 1H), 7.43 (dd, J=8.8, 2.0 Hz, 1H), 7.70 (d, J=2.0 Hz, 1H).

### Step 2—Tert-butyl 4-[4-chloro-3-(1,1-difluoroethyl)phenyl]piperazine-1-carboxylate

A mixture of 4-bromo-1-chloro-2-(1,1-difluoroethyl)benzene (2 g, 7.83 mmol), tert-butyl piperazine-1-carboxylate (1.60 g, 8.61 mmol, CAS #57260-71-6), Pd₂(dba)₃ (358 mg, 391 mol), Xantphos (498 mg, 861 μmol) and tBuONa (2.26 g, 23.5 mmol) in anhydrous dioxane (20 mL) was degassed and purged with N₂ three times. Then the mixture was stirred at 100° C. for 1 hr under N₂ atmosphere. On completion, the reaction mixture was quenched by saturated ammonium chloride in water (200 mL) and extracted with dichloromethane (2×400 mL). The combined organic layers were washed with brine (200 mL) and dried over anhydrous sodium sulfate, filtered and the filtrate was concentrated in vacuo to give the crude residue. The residue was purified by column chromatography (SiO₂, Petroleum ether:Ethyl acetate=1/0 to 10/1) to give the title compound (2.75 g, 83% yield) as yellow oil. LC-MS (ESI⁺) m/1z 305.1 (M-55)⁺; ¹H NMR (400 MHz, CDCl₃-d) 6=1.49 (s, 9H) 2.03 (t, J=18.4 Hz, 3H) 3.10-3.19 (m, 4H) 3.54-3.62 (m, 4H) 6.87 (dd, J=8.8, 2.8 Hz, 1H) 7.14 (d, J=2.8 Hz, 1H) 7.30 (d, J=8.8 Hz, 1H)

### 6-(1-Acetyl-3,6-dihydro-2H-pyridin-5-yl)-4-[2-(1,1-difluoroethyl)-4-piperazin-1-yl-phenyl]-7-fluoro-N,N-dimethyl-1H-indole-2-carboxamide (Intermediate EL)

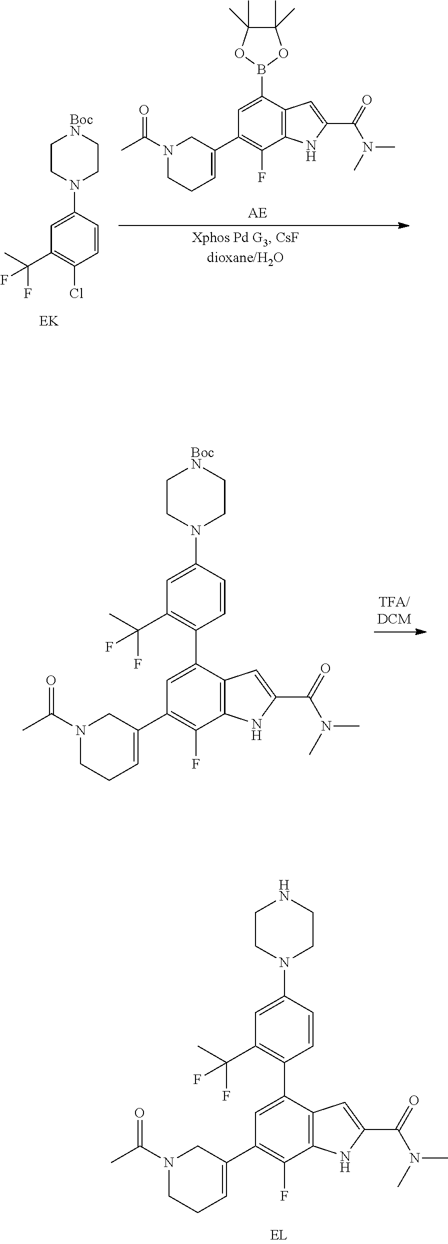

### Step 1—Tert-butyl 4-[4-[6-(1-acetyl-3,6-dihydro-2H-pyridin-5-yl)-2-(dimethylcarbamoyl)-7-fluoro-1H-indol-4-yl]-3-(1,1-difluoroethyl)phenyl]piperazine-1-carboxylate

To a solution of tert-butyl 4-[4-chloro-3-(1,1-difluoroethyl)phenyl]piperazine-1-carboxylate (500 mg, 1.18 mmol, Intermediate EK) and 6-(1-acetyl-3,6-dihydro-2H-pyridin-5-yl)-7-fluoro-N,N-dimethyl-4-(4,4,5,5-tetramethyl-1,3,2-dioxaborolan-2-yl)-1H-indole-2-carboxamide (536 mg, 1.18 mmol, Intermediate AE) in dioxane (5 mL) and water (1.25 mL) was added XPhos Pd G₃ (99.7 mg, 118 μmol) and CsF (537 mg, 3.53 mmol, 130 L). The mixture was then stirred at 80° C. for 2 hrs. On completion, the reaction mixture was diluted with water (10 mL) and extracted with ethyl acetate (20×2 mL). The combined organic layers were washed with brine (10 mL) and dried over anhydrous sodium sulfate, filtered and the filtrate was concentrated in vacuo to give the crude residue. The residue was purified by column chromatography (SiO₂, Petroleum ether:Ethyl acetate=1/0 to 0/1) to give the title compound (700 mg, 82% yield) as a brown solid. ¹H NMR (400 MHz, CDCl₃-d) 6=1.48-1.55 (m, 9H) 1.58 (s, 1H) 1.64 (s, 2H) 2.17 (s, 3H) 2.30-2.46 (m, 2H) 3.08-3.39 (m, 10H) 3.56-3.69 (m, 5H) 3.77 (br t, J=5.6 Hz, 1H) 4.26-4.43 (m, 1H) 4.48 (br s, 1H) 5.31 (s, 1H) 6.15 (br d, J=12.0 Hz, 1H) 6.44 (br d, J=7.6 Hz, 1H) 6.88 (br d, J=6.0 Hz, 1H) 6.96-7.03 (m, 1H) 7.19 (br dd, J=10.8, 8.8 Hz, 1H) 9.44-9.62 (m, 1H).

### Step 2—6-(1-Acetyl-3,6-dihydro-2H-pyridin-5-yl)-4-[2-(1,1-difluoroethyl)-4-piperazin-1-yl-phenyl]-7-fluoro-N,N-dimethyl-1H-indole-2-carboxamide

To a solution of tert-butyl 4-[4-[6-(1-acetyl-3,6-dihydro-2H-pyridin-5-yl)-2-(dimethylcarbamoyl)-7-fluoro-1H-indol-4-yl]-3-(1,1-difluoroethyl)phenyl]piperazine-1-carboxylate (600 mg, 918 μmol) in DCM (6 mL) was added TFA (3.07 g, 26.9 mmol, 2 mL). The mixture was then stirred at 25° C. for 1 hr. On completion, the mixture was concentrated in vacuo to give the title compound (600 mg) as brown oil. LC-MS (ESI⁺) m/z 554.5 (M+H)⁺.

### 3-(7-(4-(3-Methoxy-4-(4,4,5,5-tetramethyl-1,3,2-dioxaborolan-2-yl)phenyl)piperidin-1-yl)-1-methyl-1H-indazol-3-yl)piperidine-2,6-dione (Intermediate EM)

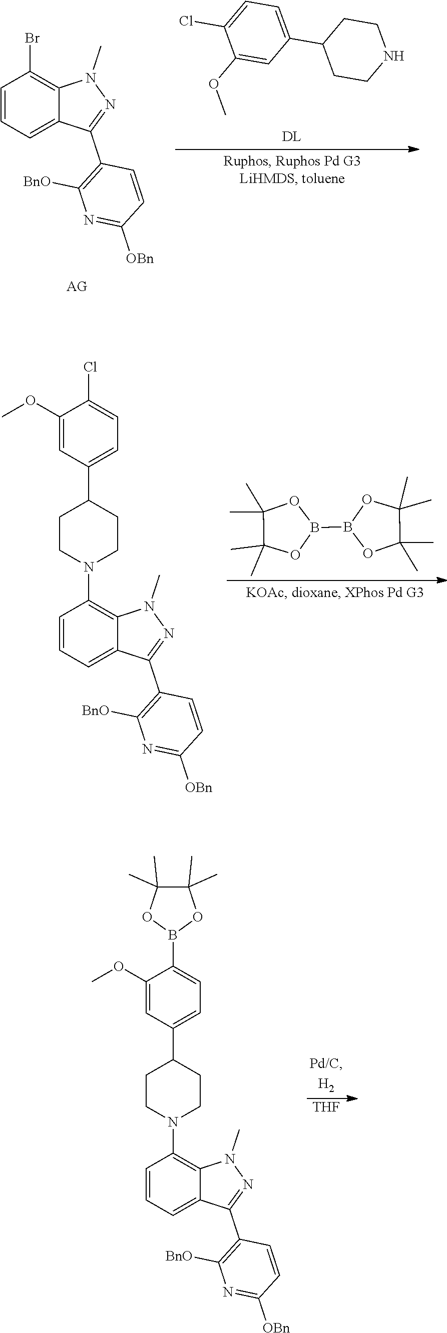

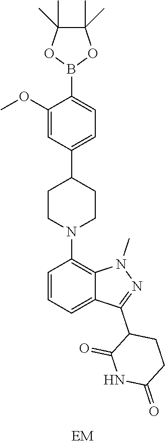 [SMILES: COc1cc(C2CCN(c3cccc4c(C5CCC(=O)NC5=O)nn(C)c34)CC2)ccc1B1OC(C)(C)C(C)(C)O1]

### Step 1—3-(2,6-Bis(benzyloxy)pyridin-3-yl)-7-(4-(4-chloro-3-methoxyphenyl)piperidin-1-yl)-1-methyl-1H-indazole

A mixture of 7-bromo-3-(2,6-dibenzyloxy-3-pyridyl)-1-methyl-indazole (1.50 g, 3.00 mmol, Intermediate AG), 4-(4-chloro-3-methoxy-phenyl)piperidine (812 mg, 3.60 mmol, Intermediate DL) and LiHMDS (1 M, 15.0 mL) in toluene (20 mL) was degassed and purged with N₂ three times. Then the mixture was stirred at 0° C. for 30 mins under N₂ atmosphere. Then RuPhos (140 mg, 300 mol) and RuPhos Pd G3 (251 mg, 300 μmol) were added and the mixture was stirred at 100° C. for 2 hrs. On completion, the reaction mixture was quenched by addition of NH₄Cl (20 mL) at 0° C., and then diluted with water (40 mL) and extracted with ethyl acetate (30 mL×2). The combined organic layers were washed with brine (20 mL×2), dried over Na₂SO₄, filtered and concentrated under reduced pressure to give a residue. The crude product was purified by reverse phase HPLC (0.1% FA condition) to give the title compound (1.3 g, 59% yield) as a white solid. LC-MS (ESI⁺) m/z 645.4 (M+H)⁺. ¹H NMR (400 MHz, CHLOROFORM-d) δ=7.81 (d, J=8.0 Hz, 1H), 7.43-7.37 (m, 3H), 7.36 (d, J=8.0 Hz, 1H), 7.33-7.31 (m, 2H), 7.30 (d, J=4.0 Hz, 2H), 7.25-7.19 (m, 4H), 7.02-6.97 (m, 1H), 6.96-6.90 (m, 1H), 6.85-6.78 (m, 2H), 6.47 (d, J=8.0 Hz, 1H), 5.42 (s, 2H), 5.35 (s, 2H), 4.42 (s, 3H), 3.90 (s, 3H), 3.41 (d, J=11.6 Hz, 2H), 2.86 (t, J=9.6 Hz, 2H), 2.65 (dt, J=5.2, 10.0 Hz, 1H), 1.98 (s, 1H), 1.54 (s, 1H), 1.21 (d, J=17.6 Hz, 2H).

### Step 2—3-(2,6-Bis(benzyloxy)pyridin-3-yl)-7-(4-(3-methoxy-4-(4,4,5,5-tetramethyl-1,3,2-dioxaborolan-2-yl)phenyl)piperidin-1-yl)-1-methyl-1H-indazole

A mixture of 7-[4-(4-chloro-3-methoxy-phenyl)-1-piperidyl]-3-(2,6-dibenzyloxy-3-pyridyl)-1-methyl-indazole (1.30 g, 1.69 mmol), 4,4,5,5-tetramethyl-2-(4,4,5,5-tetramethyl-1,3,2-dioxaborolan-2-yl)-1,3,2-dioxaborolane (2.15 g, 8.46 mmol, CAS #73183-34-3), XPhos Pd G3 (143 mg, 169 mol), and KOAc (498 mg, 5.08 mmol) in dioxane (40 mL) was degassed and purged with N₂ three times. Then the mixture was stirred at 80° C. for 2 hrs under N₂ atmosphere. On completion, the reaction mixture was filtered and concentrated under reduced pressure to give a residue. The residue was purified by column chromatography (SiO₂, Petroleum ether/Ethyl acetate=40/1 to 20/1) to give the title compound (1.00 g, 75% yield) as a white solid. LC-MS (ESI⁺) m/z 737.4 (M+H)⁺.

### Step 3—3-(7-(4-(3-Methoxy-4-(4,4,5,5-tetramethyl-1,3,2-dioxaborolan-2-yl)phenyl)piperidin-1-yl)-1-methyl-1H-indazol-3-yl)piperidine-2,6-dione

To a solution of 3-(2,6-dibenzyloxy-3-pyridyl)-7-[4-[3-methoxy-4-(4,4,5,5-tetramethyl-1,3,2-dioxaborolan-2-yl)phenyl]-1-piperidyl]-1-methyl-indazole (1.00 g, 1.36 mmol) in THF (20 mL) was added Pd/C (1.44 g, 1.36 mmol, 10 wt %) under N₂ atmosphere. The suspension was degassed and purged with H₂ three times. The mixture was then stirred under H₂ (40 Psi) at 30° C. for 12 hrs. On completion, the reaction mixture was filtered and concentrated under reduced pressure to give the title compound (450 mg) as a yellow solid. LC-MS (ESI⁺) m/z 559.1 (M+H)⁺.

### Tert-butyl 4-(3-fluoro-4-(4,4,5,5-tetramethyl-1,3,2-dioxaborolan-2-yl)phenyl)piperazine-1-carboxylate (Intermediate EN)

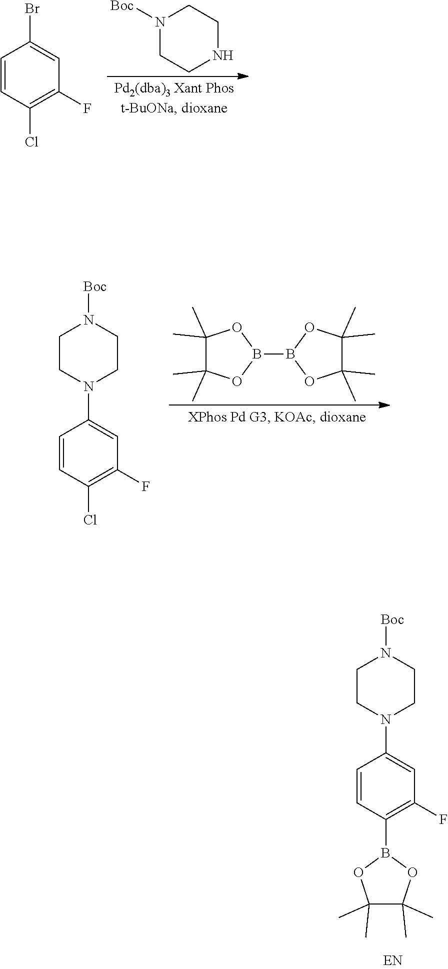

### Step 1—Tert-butyl 4-(4-chloro-3-fluorophenyl)piperazine-1-carboxylate

To a solution of 4-bromo-1-chloro-2-fluorobenzene (5 g, 23.8 mmol, CAS #60811-18-9), tert-butyl piperazine-1-carboxylate (4.89 g, 26.2 mmol, CAS #57260-71-6) in dioxane (50.0 mL) was added Pd₂(dba)₃ (1.09 g, 1.19 mmol), Xantphos (1.52 g, 2.63 mmol) and tBuONa (6.88 g, 71.6 mmol), then the reaction was stirred at 100° C. for 1 hr under nitrogen atmosphere. On completion, the reaction mixture was diluted with water (50.0 mL) and extracted with ethyl acetate (3×200 mL). The combined organic layers were washed with brine (50.0 mL) and dried over anhydrous sodium sulfate, filtered and the filtrate was concentrated in vacuo to give the crude residue. The crude residue was purified by column chromatography (SiO₂, Petroleum ether/Ethyl acetate=1/0) to give the title compound (7.00 g, 85% yield) as a yellow solid. LC-MS (ESI⁺) m/z 315.0 (M+H)⁺.

### Step 2—Tert-butyl 4-(3-fluoro-4-(4,4,5,5-tetramethyl-1,3,2-dioxaborolan-2-yl)phenyl)piperazine-1-carboxylate

To a solution of tert-butyl 4-(4-chloro-3-fluorophenyl)piperazine-1-carboxylate (2 g, 6 mmol), 4,4,4′,4′,5,5,5′,5′-octamethyl-2,2′-bi(1,3,2-dioxaborolane) (4.84 g, 19.6 mmol, CAS #73183-34-3) in dioxane (20.0 mL) was added KOAc (1.87 g, 19.0 mmol) and XPhos Pd G3 (537 mg, 635 μmol), then the reaction was stirred at 80° C. for 2 hrs under nitrogen atmosphere. On completion, the reaction mixture was diluted with water (50.0 mL) and extracted with ethyl acetate (3×100 mL). The combined organic layers were washed with brine (50.0 mL) and dried over anhydrous sodium sulfate, filtered and the filtrate was concentrated in vacuo to give the crude residue. The crude residue was purified by reverse phase HPLC (0.8 g/L ammonium bicarbonate) to give the title compound (1.40 g, 52% yield) as a white solid. LC-MS (ESI⁺) m/z 407.2 (M+H)⁺.

### 7-Fluoro-4-(2-fluoro-4-(piperazin-1-yl)phenyl)-6-(1-isobutyryl-1,2,5,6-tetrahydropyridin-3-yl)-N,N-dimethyl-1H-indole-2-carboxamide (Intermediate EO)

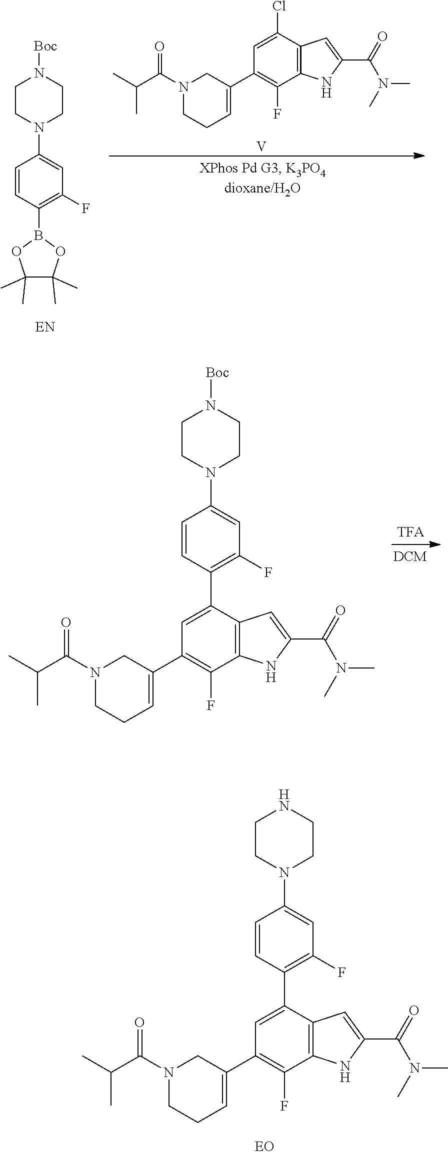

### Step 1—Tert-butyl 4-(4-(2-(dimethylcarbamoyl)-7-fluoro-6-(1-isobutyryl-1,2,5,6-tetrahydropyridin-3-yl)-1H-indol-4-yl)-3-fluorophenyl)piperazine-1-carboxylate

To a solution of tert-butyl 4-(3-fluoro-4-(4,4,5,5-tetramethyl-1,3,2-dioxaborolan-2-yl)phenyl)piperazine-1-carboxylate (400 mg, 984 μmol, Intermediate EN), 4-chloro-7-fluoro-6-(1-isobutyryl-1,2,5,6-tetrahydropyridin-3-yl)-N,N-dimethyl-1H-indole-2-carboxamide (347 mg, 886 μmol, Intermediate V) in dioxane (4.00 mL), H₂O (1.20 mL) was added K₃PO₄ (626 mg, 2.95 mmol), and XPhos Pd G3 (83.3 mg, 98.4 mol). Then the reaction was stirred at 80° C. for 0.5 hrs under nitrogen atmosphere. On completion, the reaction mixture was diluted with water (50 mL) and extracted with ethyl acetate (3×200 mL). The combined organic layers were washed with brine (50 mL) and dried over anhydrous sodium sulfate, filtered and the filtrate was concentrated in vacuo to give the crude residue. The crude residue was purified by column chromatography (SiO₂, Petroleum ether/Ethyl acetate=10/0 to 0/1) to give the title compound (500 mg, 78% yield) as a yellow solid. LC-MS (ESI⁺) m/z 636.2 (M+H)⁺.

### Step 2—7-Fluoro-4-(2-fluoro-4-(piperazin-1-yl)phenyl)-6-(I-isobutyryl-1,2,5,6-tetrahydropyridin-3-yl)-N,N-dimethyl-1H-indole-2-carboxamide

To a solution of tert-butyl 4-(4-(2-(dimethylcarbamoyl)-7-fluoro-6-(1-isobutyryl-1,2,5,6-tetrahydropyridin-3-yl)-1H-indol-4-yl)-3-fluorophenyl)piperazine-1-carboxylate (500 mg, 786 mol) in DCM (4.00 mL) was added TFA (1.54 g, 13.4 mmol, 1.00 mL). The mixture was then stirred at 25° C. for 1 hr. On completion, the reaction mixture was concentrated in vacuo to give the crude residue. The crude residue was purified by reverse phase HPLC (0.8 g/L ammonium bicarbonate) to give the title compound (320 mg, 75% yield) as a white solid. LC-MS (ESI⁺) n/z 536.2 (M+H)⁺.

### 4-(2-Chloro-5-fluoro-4-(piperazin-1-yl)phenyl)-7-fluoro-6-(1-isobutyryl-1,2,5,6-tetrahydropyridin-3-yl)-N,N-dimethyl-1H-indole-2-carboxamide (Intermediate EP)

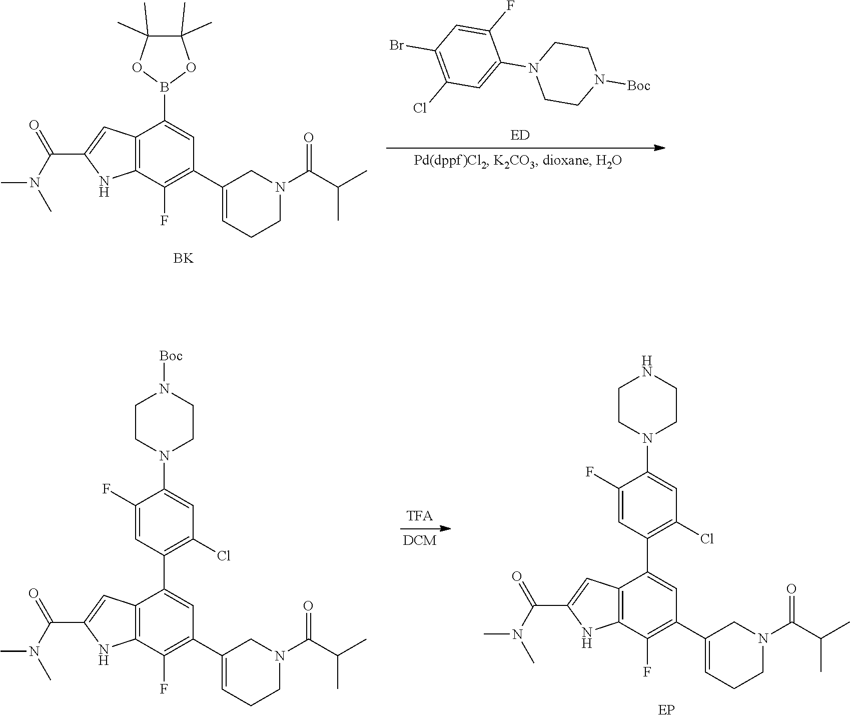

### Step 1—Tert-butyl 4-(5-chloro-4-(2-(dimethylcarbamoyl)-7-fluoro-6-(1-isobutyryl-1,2,5,6-tetrahydropyridin-3-yl)-1H-indol-4-yl)-2-fluorophenyl)piperazine-1-carboxylate

To a solution of tert-butyl 4-(4-bromo-5-chloro-2-fluorophenyl)piperazine-1-carboxylate (500 mg, 1.27 mmol, Intermediate ED) in dioxane (10 mL) and H₂O (2 mL) was added 7-fluoro-6-(1-isobutyryl-1,2,5,6-tetrahydropyridin-3-yl)-N,N-dimethyl-4-(4,4,5,5-tetramethyl-1,3,2-dioxaborolan-2-yl)-1H-indole-2-carboxamide (675 mg, 1.40 mmol, Intermediate BK), K₂CO₃ (526 mg, 3.81 mmol) and Pd(dppf)Cl₂ (92.9 mg, 127 μmol). The reaction was then stirred at 80° C. for 2 hrs. On completion, the reaction mixture was concentrated in vacuo to give the title compound (800 mg) as a brown solid. LC-MS (ESI⁺) m/z 670.3. (M+H)⁺.

### Step 2—4-(2-Chloro-5-fluoro-4-(piperazin-1-yl)phenyl)-7-fluoro-6-(1-isobutyryl-1,2,5,6-tetrahydropyridin-3-yl)-N,N-dimethyl-1H-indole-2-carboxamide

To a solution of tert-butyl 4-(5-chloro-4-(2-(dimethylcarbamoyl)-7-fluoro-6-(1-isobutyryl-1,2,5,6-tetrahydropyridin-3-yl)-1H-indol-4-yl)-2-fluorophenyl)piperazine-1-carboxylate (800 mg, 1.19 mmol) in DCM (8 mL) was added TFA (6.14 g, 53.8 mmol, 4 mL). The reaction was stirred at 25° C. for 2 hrs. On completion, the reaction mixture was concentrated in vacuo to give the crude residue. The crude residue was purified by reverse phase HPLC (0.1% FA condition) to give the title compound (500 mg, 73% yield) as a white solid. LC-MS (ESI⁺) m/z 570.0. (M+H)⁺.

### 3-(4-(4-(4-Chlorophenyl)piperidin-1-yl)-3-methyl-1H-indazol-1-yl)piperidine-2,6-dione (Intermediate EQ)

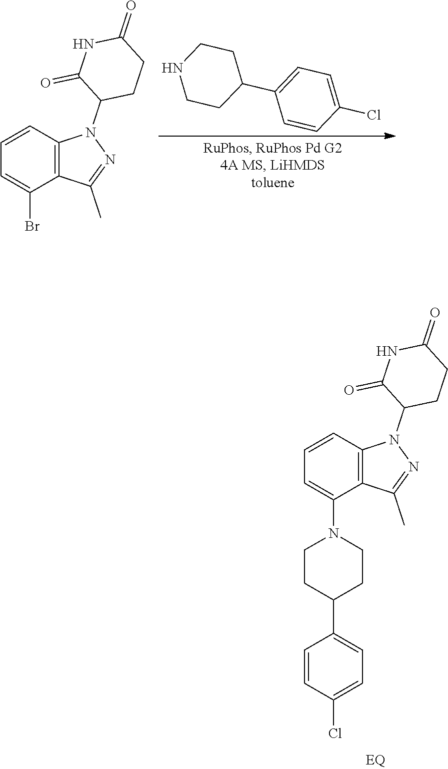

To a solution of 3-(4-bromo-3-methyl-1H-indazol-1-yl)piperidine-2,6-dione (1 g, 3.10 mmol, synthesized via Step 1 of Intermediate AB), 4-(4-chlorophenyl)piperidine (607 mg, 3.10 mmol, CAS #26905-02-2) and 4A molecular sieves (2 g) in toluene (20 mL) was added LiHMDS (1 M, 18.6 mL) at 0° C. After 30 minutes, RuPhos (217 mg, 466 μmol) and RuPhos Pd G2 (241 mg, 310 mol) was added and the mixture was stirred at 100° C. for 3 hrs. On completion, the reaction mixture was concentrated under reduced pressure to remove solvent. Then the crude product was purified by reverse phase HPLC (neutral condition) to give the title compound (70 mg, 4% yield) as a yellow solid. LC-MS (ESI⁺) m/z 437.1 (M+H)⁺.

### Tert-butyl 4-(4-bromo-2,5-difluorophenyl)piperazine-1-carboxylate (Intermediate ER)

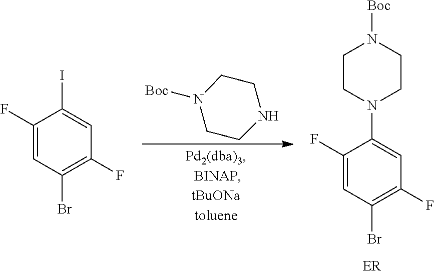 [SMILES: Fc1cc(I)c(F)cc1Br ; CC(C)(C)OC(=O)N1CCNCC1 ; CC(C)(C)OC(=O)N1CCN(c2cc(F)c(Br)cc2F)CC1]

A mixture of 1-bromo-2,5-difluoro-4-iodobenzene (5 g, 20 mmol, CAS #145349-66-2), tert-butyl piperazine-1-carboxylate (2.92 g, 15.6 mmol, CAS #57260-71-6), BINAP (1.95 g, 3.14 mmol), t-BuONa (3.01 g, 31.3 mmol) and Pd₂(dba)₃ (1.44 g, 1.57 mmol) in toluene (50 mL) was degassed and purged with N₂ three times. Then the mixture was stirred at 90° C. for 12 hrs under N₂ atmosphere. On completion, the reaction mixture was concentrated in vacuo and purified by column chromatography (SiO₂, Petroleum ether/Ethyl acetate=1/0 to 5/1) to give the title compound (3.5 g, 45% yield) as a red solid. LC-MS (ESI⁺) m/z 279.9 (M+1)⁺.

### 4-(2,5-Difluoro-4-(piperazin-1-yl)phenyl)-7-fluoro-6-(1-isobutyryl-1,2,5,6-tetrahydropyridin-3-yl)-N,N-dimethyl-1H-indole-2-carboxamide (Intermediate ES)

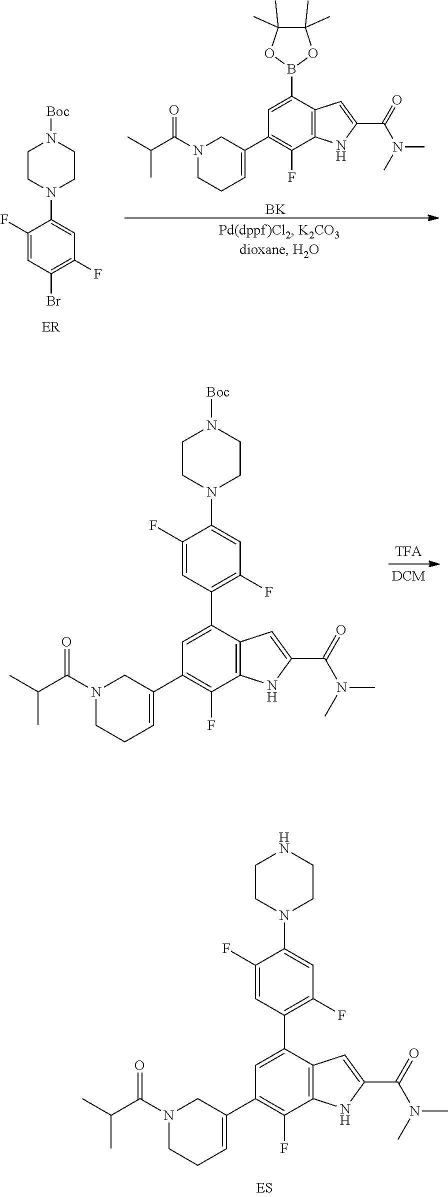

### Step 1—Tert-butyl 4-(4-(2-(dimethylcarbamoyl)-7-fluoro-6-(1-isobutyryl-1,2,5,6-tetrahydropyridin-3-yl)-1H-indol-4-yl)-2,5-difluorophenyl)piperazine-1-carboxylate

A mixture of tert-butyl 4-(4-bromo-2,5-difluorophenyl)piperazine-1-carboxylate (300 mg, 795 μmol, Intermediate ER), 7-fluoro-6-(1-isobutyryl-1,2,5,6-tetrahydropyridin-3-yl)-N,N-dimethyl-4-(4,4,5,5-tetramethyl-1,3,2-dioxaborolan-2-yl)-1H-indole-2-carboxamide (384 mg, 795 μmol, Intermediate BK), K₂CO₃ (329 mg, 2.39 mmol), and Pd(dppf)Cl₂ (64.9 mg, 79.5 μmol) in dioxane (10 mL) and H₂O (2.5 mL) was degassed and purged with N₂ three times. Then the mixture was stirred at 80° C. for 2 hrs under N₂ atmosphere. On completion, the reaction mixture was triturated with H₂O at 0° C. for 15 min, then filtered to give the title compound (400 mg) as a white solid. LC-MS (ESI⁺) m/z 598.2 (M−55)⁺.

### Step 2—4-(2,5-Difluoro-4-(piperazin-1-yl)phenyl)-7-fluoro-6-(1-isobutyryl-1,2,5,6-tetrahydropyridin-3-yl)-N,N-dimethyl-1H-indole-2-carboxamide

To a solution of tert-butyl 4-(4-(2-(dimethylcarbamoyl)-7-fluoro-6-(1-isobutyryl-1,2,5,6-tetrahydropyridin-3-yl)-1H-indol-4-yl)-2,5-difluorophenyl)piperazine-1-carboxylate (300 mg, 458 μmol) in DCM (3 mL) was added TFA (52 mg, 458 μmol, 34.0 L). The mixture was then stirred at 25° C. for 2 hrs. On completion, the reaction mixture was concentrated in vacuo to give the title compound (250 mg, TFA) as a red solid. LC-MS (ESI⁺) m/z 554.5 (M+1)⁺.

### 1-(4-Chloro-3-fluorophenyl)piperazine (Intermediate ET)

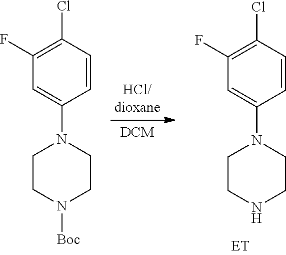 [SMILES: CC(C)(C)OC(=O)N1CCN(c2ccc(Cl)c(F)c2)CC1 ; Fc1cc(N2CCNCC2)ccc1Cl]

To a solution of tert-butyl 4-(4-chloro-3-fluorophenyl)piperazine-1-carboxylate (1.5 g, 4.77 mmol, synthesized via Step 1 of Intermediate EN) in DCM (10 mL) was added HCl/dioxane (2 M, 10 mL). The mixture was then stirred at 25° C. for 8 hrs. On completion, the reaction mixture was concentrated under reduced pressure to give the title compound (1.2 g) as a white solid. LC-MS (ESI⁺) m/z 214.9 (M+H)⁺.

### 3-((4-(4-(4-Chloro-3-fluorophenyl)piperazin-1-yl)-5-fluoro-2-methoxyphenyl)amino)piperidine-2,6-dione (Intermediate EU)

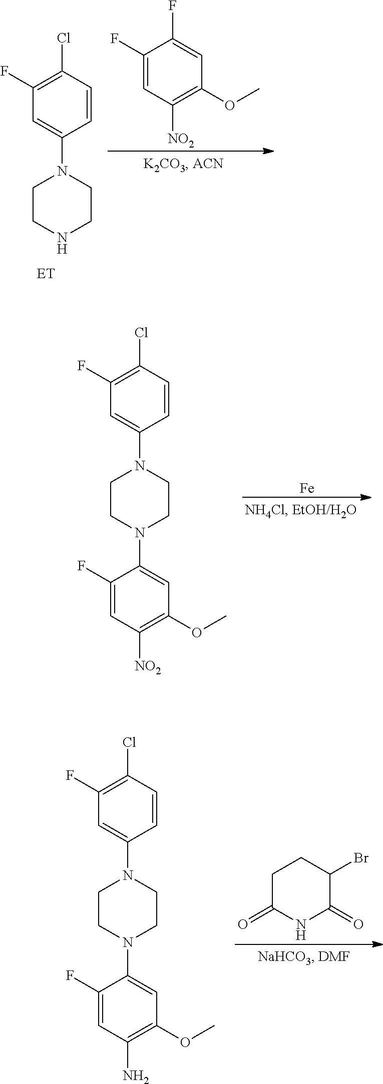

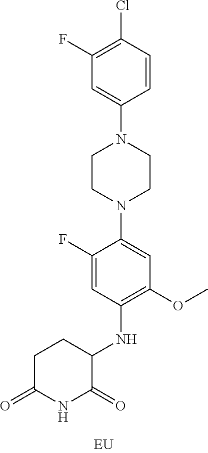 [SMILES: COc1cc(N2CCN(c3ccc(Cl)c(F)c3)CC2)c(F)cc1NC1CCC(=O)NC1=O]

### Step 1—1-(4-Chloro-3-fluorophenyl)-4-(2-fluoro-5-methoxy-4-nitrophenyl)piperazine

To a solution of 1-(4-chloro-3-fluorophenyl)piperazine (1.2 g, 5.59 mmol, Intermediate ET) and 1,2-difluoro-4-methoxy-5-nitrobenzene (1.06 g, 5.59 mmol, synthesized via Step 1 of Intermediate AQ) in ACN (10 mL) was added K₂CO₃ (2.32 g, 16.7 mmol). The mixture was then stirred at 80° C. for 2 hrs. On completion, the reaction mixture was concentrated under reduced pressure to give a residue. The residue was purified by column chromatography (SiO₂, Petroleum ether:Ethyl acetate=1/0 to 5/1) to give the title compound (1.5 g, 68% yield) as a yellow solid. LC-MS (ESI⁺) m, z 384.0 (M+H)⁺.

### Step 2—4-(4-(4-Chloro-3-fluorophenyl)piperazin-1-yl)-5-fluoro-2-methoxyaniline

To a solution of 1-(4-chloro-3-fluorophenyl)-4-(2-fluoro-5-methoxy-4-nitrophenyl)piperazine (1 g. 2.61 mmol) in EtOH (10 mL) and H₂O (10 mL) was added Fe (1.02 g, 18.2 mmol) and NH₄Cl (975 mg, 18.2 mmol). The mixture was then stirred at 50° C. for 3 hrs. On completion, the reaction mixture was filtered to give the filtrate and the reaction mixture was concentrated under reduced pressure to give the title compound (1 g) as a brown solid. LC-MS (ESI⁺) m/z 354.0 (M+H)⁺.

### Step 3—3-((4-(4-(4-Chloro-3-fluorophenyl)piperazin-1-yl)-5-fluoro-2-methoxyphenyl)amino)piperidine-2,6-dione

To a solution of 4-(4-(4-chloro-3-fluorophenyl)piperazin-1-yl)-5-fluoro-2-methoxyaniline (0.6 g, 1.70 mmol) and 3-bromopiperidine-2,6-dione (651 mg, 3.39 mmol, CAS #62595-74-8) in DMF (5 mL) was added NaHCO₃ (427 mg, 5.09 mmol), then the mixture was stirred at 80° C. for 12 hrs. On completion, the reaction mixture was concentrated under reduced pressure to give a residue. The residue was purified by column chromatography (SiO₂, Petroleum ether:Ethyl acetate=10/1 to 1/1) to give the title compound (0.2 g, 23% yield) as a brown solid. LC-MS (ESI⁺) m/z 464.7 (M+H)⁺.

### 1-(4-Bromo-2,5-difluorophenyl)piperazine (Intermediate EV)

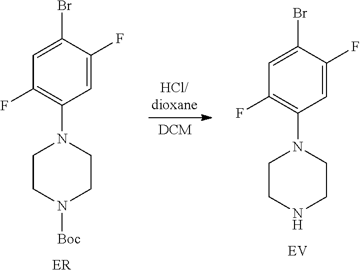 [SMILES: CC(C)(C)OC(=O)N1CCN(c2cc(F)c(Br)cc2F)CC1 ; Fc1cc(N2CCNCC2)c(F)cc1Br]

To a solution of tert-butyl 4-(4-bromo-2,5-difluorophenyl)piperazine-1-carboxylate (1 g, 2.65 mmol, Intermediate ER) in DCM (10 mL) was added HCl/dioxane (2 M. 1.33 mL). The mixture was then stirred at 25° C. for 2 hrs. On completion, the reaction mixture was filtered was concentrated in vacuo to give the title compound (0.8 g) as a yellow solid. LC-MS (ESI⁺) m/z 276.9 (M−H)⁻.

### 4-(4-(4-Bromo-2,5-difluorophenyl)-piperazin-1-yl)-5-fluoro-2-methoxyaniline (Intermediate EW)

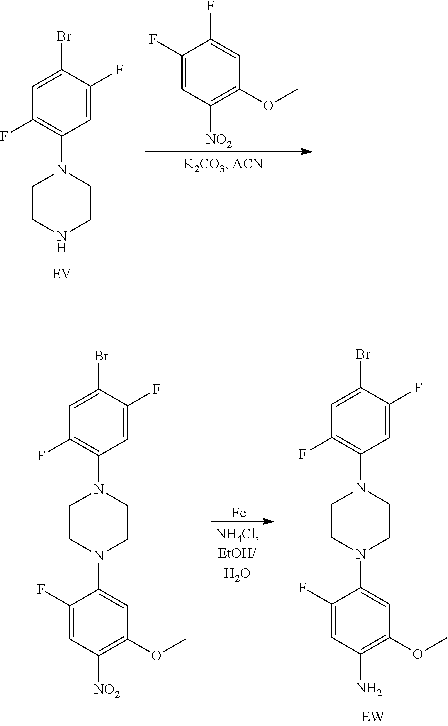

### Step 1—1-(4-Bromo-2,5-difluorophenyl)-4-(2-fluoro-5-methoxy-4-nitrophenyl)piperazine

To a solution of 1,2-difluoro-4-methoxy-5-nitrobenzene (477 mg, 2.53 mmol, CAS #66684-64-8) in DMF (3 mL) was added K₂CO₃ (1.05 g, 7.58 mmol) and 1-(4-bromo-2,5-difluorophenyl)piperazine (0.7 g, 2.53 mmol, Intermediate EV). The mixture was then stirred at 80° C. for 2 hrs. On completion, the reaction mixture was filtered and the filtrate was concentrated in vacuo to give the crude residue. The residue was purified by column chromatography (SiO₂. Petroleum ether:Ethyl acetate=1/0 to 1/1) to give the title compound (0.5 g) as a yellow solid. LC-MS (ESI⁺) m/z 445.8 (M−+H)⁺.

### Step 2—4-(4-(4-Bromo-2,5-difluorophenyl)piperazin-1-yl)-5-fluoro-2-methoxyaniline

To a solution of 1-(4-bromo-2,5-difluorophenyl)-4-(2-fluoro-5-methoxy-4-nitrophenyl)piperazine (0.5 g, 1.12 mmol) in EtOH (5 mL) was added Fe (438 mg, 7.84 mmol) and NH₄Cl (419 mg, 7.84 mmol). The mixture was then stirred at 80° C. for 2 hrs. On completion, the reaction mixture was filtered and the filtrate was concentrated in vacuo to give the crude residue (0.4 g, crude) as a yellow solid. LC-MS (ESI⁺) m/z 416.1 (M+H)⁺.

### 3-((4-(4-(4-Chloro-2,5-difluorophenyl)piperazin-1-yl)-5-fluoro-2-methoxyphenyl)amino)piperidine-2,6-dione (Intermediate EX)

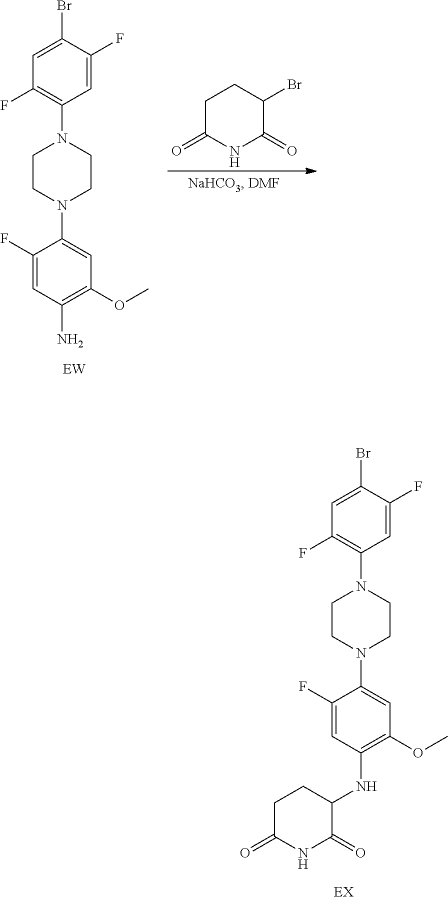 [SMILES: COc1cc(N2CCN(c3cc(F)c(Br)cc3F)CC2)c(F)cc1N ; O=C1CCC(Br)C(=O)N1 ; COc1cc(N2CCN(c3cc(F)c(Br)cc3F)CC2)c(F)cc1NC1CCC(=O)NC1=O]

To a solution of 3-bromopiperidine-2,6-dione (206 mg, 1.08 mmol, CAS #62595-74-8) in DMF (3 mL) was added NaHCO₃ (90.3 mg, 1.08 mmol) and 4-(4-(4-bromo-2,5-difluorophenyl)piperazin-1-yl)-5-fluoro-2-methoxyaniline (223 mg, 537 μmol, Intermediate EW). The mixture was then stirred at 90° C. for 2 hrs. On completion, the reaction mixture was filtered and the filtrate was concentrated in vacuo to give the crude residue. The residue was purified by column chromatography (SiO₂, Petroleum ether:Ethyl acetate=1/0 to 0/1) to give the title compound (0.1 g, 35% yield) as a green solid. LC-MS (ESI⁺) m/z 528.0 (M+H)⁺.

### 3-(3-Fluoro-4-(4-(3-methoxy-4-(4,4,5,5-tetramethyl-1,3,2-dioxaborolan-2-yl)phenyl)piperidin-1-yl)phenyl)piperidine-2,6-dione (Intermediate EY)

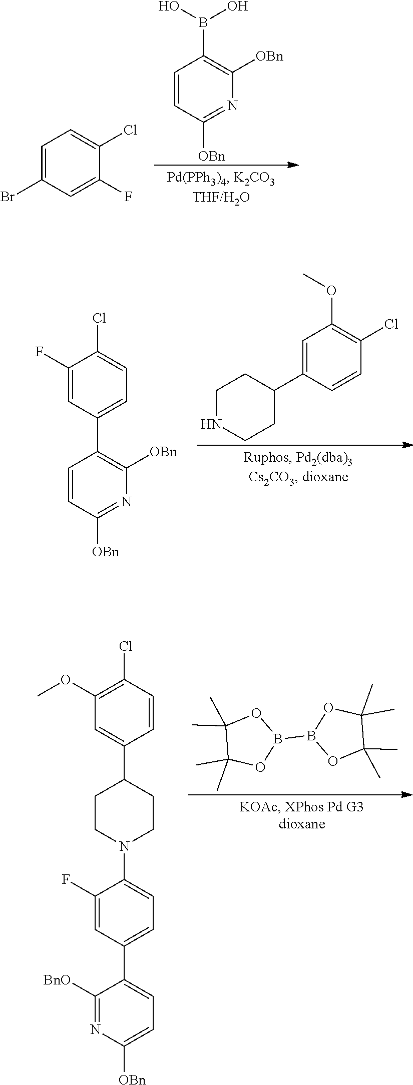

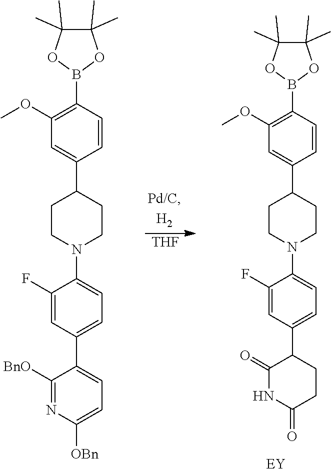

### Step 1—2,6-Bis(benzyloxy)-3-(4-chloro-3-fluorophenyl)pyridine

To a solution of 4-bromo-1-chloro-2-fluorobenzene (2 g, 10 mmol, CAS #60811-18-9) and (2,6-dibenzyloxy-3-pyridyl)boronicacid (3.2 g, 9.6 mmol, CAS #2096339-92-1) in THF (40 mL) and H₂O (10 mL) was added Pd(PPh₃)₄ (1.1 g, 955 μmol) and K₂CO₃ (3.96 g. 28.7 mmol). The mixture was then stirred at 60° C. for 12 hrs. On completion, the mixture was diluted with water (50 mL) and extracted with ethyl acetate (50 mL×2), then the combined organic layer was dried over sodium sulfate, filtered and concentrated under reduced pressure. The residue was purified by silica gel column chromatography (petroleum ether/ethyl acetate=1/0˜50/1). to give the title compound (2 g, 36% yield) as a yellow oil. LC-MS (ESI⁺) m/z 420.0 (M+H)⁺.

### Step 2—2,6-Bis(benzyloxy)-3-(4-(4-(4-chloro-3-methoxyphenyl)piperidin-1-yl)-3-fluorophenyl)pyridine

To a solution of 2,6-bis(benzyloxy)-3-(4-chloro-3-fluorophenyl)pyridine (800 mg, 1.91 mmol), 4-(4-chloro-3-methoxyphenyl)piperidine (473 mg, 2.10 mmol, CAS #2168546-59-4) in dioxane (15 mL) was added Cs₂CO₃ (621 mg, 1.91 mmol) at 0° C., and the mixture was stirred at 0° C. for 0.5 hr. Then Pd₂(dba)₃ (110 mg, 191 μmol) and Ruphos (178 mg, 381 μmol) was added and the mixture was degassed and purged with N₂ three times. Then the mixture was stirred at 100° C. for 4 hrs. On completion, the mixture was diluted with water (50 mL) and extracted with ethyl acetate (50 mL×2), and the combined organic layer was dried over sodium sulfate, filtered and concentrated under reduced pressure. The residue was purified by silica gel column chromatography (petroleum ether/ethyl acetate=1/0˜10/1) give the title compound (215 mg. 21% yield) as a yellow solid. LC-MS (ESI⁺) m/z 609.0 (M+H)⁺.

### Step 3—2,6-Bis(benzyloxy)-3-(3-fluoro-4-(4-(3-methoxy-4-(4,4,5,5-tetramethyl-1,3,2-dioxaborolan-2-yl)phenyl)piperidin-1-yl)phenyl)pyridine

To a solution of 2,6-bis(benzyloxy)-3-(4-(4-(4-chloro-3-methoxyphenyl)piperidin-1-yl)-3-fluorophenyl)pyridine (375 mg, 615 μmol), 4,4,4′,4′,5,5,5′,5′-octamethyl-2,2′-bi(1,3,2-dioxaborolane) (313 mg, 1.23 mmol), Xphos Pd G3 (52.1 mg, 61.6 μmol), and KOAc (181 mg, 1.85 mmol) in dioxane (5 mL) was degassed and purged with N₂ three times. Then the mixture was stirred at 80° C. for 2 hrs under N₂ atmosphere. On completion, the mixture was diluted with water (50 mL) and extracted with ethyl acetate (50 mL×2), and the combined organic layer was dried over sodium sulfate, filtered and concentrated under reduced pressure. The residue was purified by silica gel column chromatography (petroleum ether/ethyl acetate=10/1˜3/1) to give the title compound (268 mg, 60% yield) as a yellow solid. LC-MS (ESI⁺) m/z 700.9 (M+H)⁺.

### Step 4—3-(3-Fluoro-4-(4-(3-methoxy-4-(4,4,5,5-tetramethyl-1,3,2-dioxaborolan-2-yl)phenyl)piperidin-1-yl)phenyl)piperidine-2,6-dione

To a solution of 2,6-bis(benzyloxy)-3-(3-fluoro-4-(4-(3-methoxy-4-(4,4,5,5-tetramethyl-1,3,2-dioxaborolan-2-yl)phenyl)piperidin-1-yl)phenyl)pyridine (400 mg, 552 μmol) in THF (6 mL) was added Pd/C (400 mg, 305 μmol 10 wt %), then the mixture was stirred at 30° C. for 12 hrs under H₂ atmosphere (40 Psi). On completion, the mixture was filtered and concentrated under reduced pressure to give the title compound (150 mg) as a white solid. LC-MS (ESI⁺) m/z 523.2 (M+H)⁺.

### 3-(3-Methyl-2-oxo-4-(4-oxopiperidin-1-yl)-2,3-dihydro-1H-benzo[d]imidazol-1-yl)piperidine-2,6-dione (Intermediate EZ)

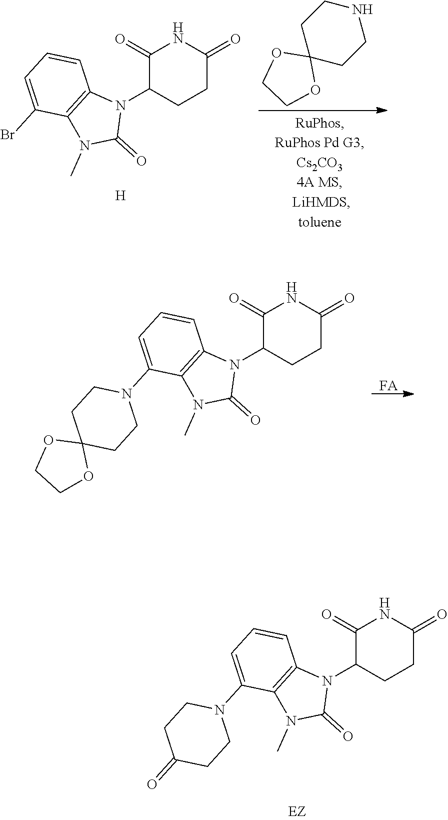

### Step 1—3-(3-Methyl-2-oxo-4-(1,4-dioxa-8-azaspiro[4.5]decan-8-yl)-2,3-dihydro-1H-benzo[d]imidazol-1-yl)piperidine-2,6-dione

To as solution of 3-(4-bromo-3-methyl-2-oxo-2,3-dihydro-1H-benzo[d]imidazol-1-yl)piperidine-2,6-dione (1 g, 2.96 mmol. Intermediate H) in toluene (15 mL) was added 1,4-dioxa-8-azaspiro[4.5]decane (635 mg, 4.44 mmol, 568 μL, CAS #177-11-7) and 4 Å molecular sieves (10 mg). The mixture was degassed and purged with nitrogen before RuPhos (137 mg, 295 μmol), RuPhos Pd G3 (247 mg, 295.72 μmol) and LiHMDS (1 M, 14.8 mL) was added. The mixture was then heated to 100° C., and stirred for 1 hr. On completion, the reaction mixture was quenched with water (20 mL) and extracted with dichloromethane (2×20 mL). The combined organic layers were washed with brine (20 mL) and dried over anhydrous sodium sulfate, filtered and the filtrate was concentrated in vacuo to give the crude residue. The residue was purified by column chromatography (SiO₂, Petroleum ether:Ethyl acetate=l/1 to 0/1) to give the title compound (500 mg, 30% yield) as a white solid. LC-MS (ESI⁺) m/z 401.1 (M+H)⁺.

### Step 2—3-(3-Methyl-2-oxo-4-(4-oxopiperidin-1-yl)-2,3-dihydro-1H-benzo[d]imidazol-1-yl)piperidine-2,6-dione

A solution of 3-(3-methyl-2-oxo-4-(1,4-dioxa-8-azaspiro[4.5]decan-8-yl)-2,3-dihydro-1H-benzo[d]imidazol-1-yl)piperidine-2,6-dione (250 mg, 437 μmol) in formic acid (1 mL) was stirred at 25° C. for 12 hrs. On completion, the mixture was concentrated under reduced pressure and was purified by reverse phase HPLC (0.1% FA condition) to give the title compound (100 mg, 64% yield) as a yellow solid. LC-MS (ESI⁺) m/z 357.1 (M+H)⁺.

### 3-(4-(4-(4-(4-Bromophenyl)piperazin-1-yl)piperidin-1-yl)-3-methyl-2-oxo-2,3-dihydro-1H-benzo[d]imidazol-1-yl)piperidine-2,6-dione (Intermediate FA)

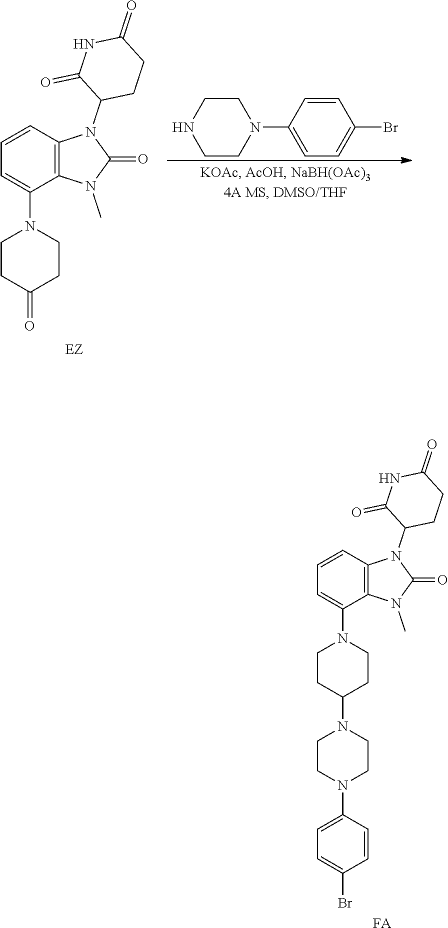

To a solution of 1-(4-bromophenyl)piperazine (150 mg, 540 μmol, CAS #66698-28-0) in THF (1 mL) and DMSO (0.5 mL) was added KOAc (79.6 mg, 810 μmol) at 25° C. After the addition, 3-(3-methyl-2-oxo-4-(4-oxopiperidin-1-yl)-2,3-dihydro-1H-benzo[d]imidazol-1-yl)piperidine-2,6-dione (217 mg, 540 μmol, Intermediate EZ), 4 Å molecular sieves (200 mg) and HOAc (64.9 mg, 1.08 mmol) was added to the mixture at 25° C. The resulting mixture was stirred at 60° C. for 12 hrs. Then the NaBH(OAc)₃ (343 mg, 1.62 mmol) was added at 0° C., and the resulting mixture was stirred at 25° C. for 2 hrs. On completion, the reaction mixture was filtered and the filtrate was concentrated in vacuo to give the crude residue. The crude residue was purified by reverse phase HPLC (0.8 g/L ammonium bicarbonate) to give the title compound (100 mg, 31% yield) as a white solid. LC-MS (ESI⁺) m/z 582.9 (M+H)⁺.

### 1-(4-Bromo-5-chloro-2-fluoro-phenyl) piperazine (Intermediate FB)

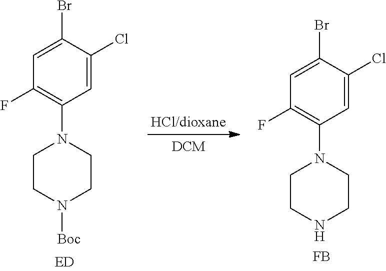

To a solution of tert-butyl 4-(4-bromo-5-chloro-2-fluoro-phenyl) piperazine-1-carboxylate (0.5 g, 1.27 mmol, Intermediate ED) in DCM (12 mL) was added HCl/dioxane (4 M, 3 mL). The mixture was then stirred at 25° C. for 2 hrs. On completion, the reaction mixture was concentrated under reduced pressure to remove solvent and give the title compound (0.5 g) as a yellow solid. LC-MS (ESI⁺) m/z 294.9 (M+H)⁺.

### 3-(4-(2-(4-(4-Bromo-5-chloro-2-fluorophenyl)piperazin-1-yl)ethyl)-3-methyl-2-oxo-2,3-dihydro-1H-benzo[d]imidazol-1-yl)piperidine-2,6-dione (Intermediate FC)

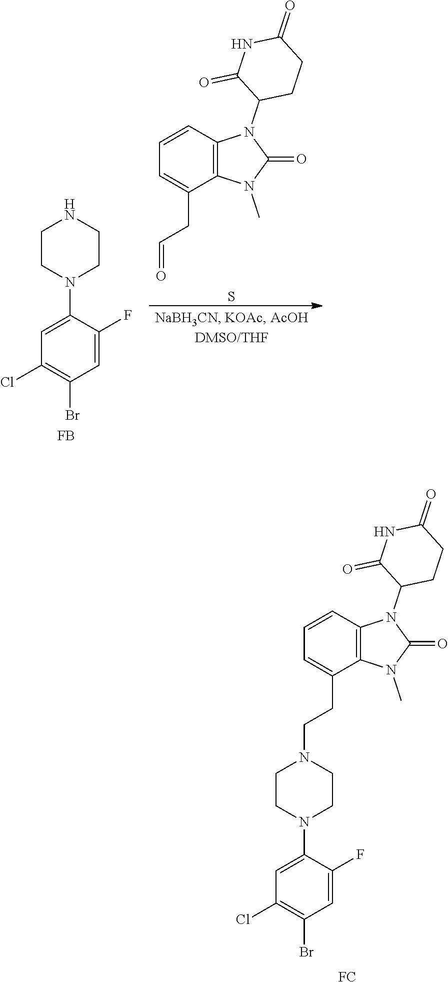

To a solution of 1-(4-bromo-5-chloro-2-fluorophenyl)piperazine (1.30 g, 4.43 mmol, Intermediate FB) and 2-(1-(2,6-dioxopiperidin-3-yl)-3-methyl-2-oxo-2,3-dihydro-1H-benzo[d]imidazol-4-yl)acetaldehyde (1.33 g. 4.43 mmol, Intermediate S) in DMSO (10.0 mL) and THF (10.0 mL) was added AcOH (797 mg, 13.2 mmol, 760 L) dropwise, and KOAc (1.30 g, 13.2 mmol) at 25° C. After addition, the mixture was stirred at rt for 1 hr, and then NaBH₃CN (556 mg, 8.86 mmol) was added. The resulting mixture was stirred at 25° C. for 1 hr. On completion, the reaction mixture was filtered and the filter cake was dried in vacuo to give the title compound (2 g) as a white solid. LC-MS (ESI⁺) m/z 578.1 (M+H)⁻.

### 4-[4-(4-Bromo-5-chloro-2-fluoro-phenyl)piperazin-1-yl]-5-fluoro-2-methoxy-aniline (Intermediate FD)

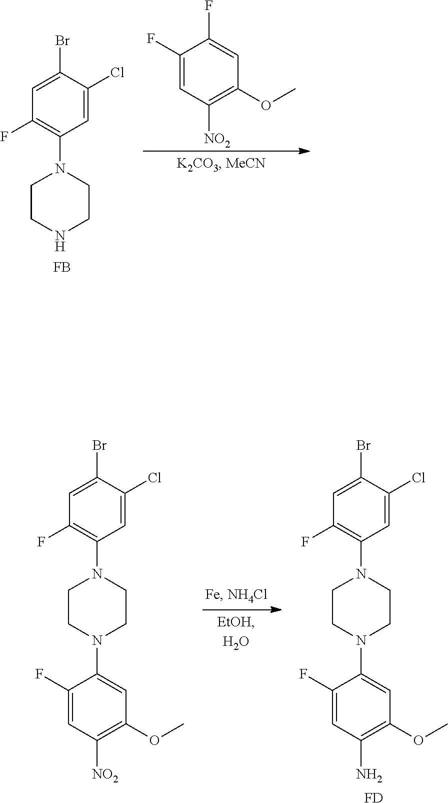

### Step 1—1-(4-Bromo-5-chloro-2-fluoro-phenyl)-4-(2-fluoro-5-methoxy-4-nitro-phenyl) piperazine

To a solution of 1-(4-bromo-5-chloro-2-fluoro-phenyl)piperazine (350 mg, 1.06 mmol, Intermediate FB) and 1,2-difluoro-4-methoxy-5-nitro-benzene (241 mg, 1.27 mmol, CAS #66684-64-8) in MeCN (6 mL) was added K₂CO₃ (440 mg, 3.18 mmol). The mixture was then stirred at 80° C. for 12 hrs. On completion, the reaction mixture was diluted with water (200 mL) and extracted with dichloromethane (300×2 mL). The combined organic layers were washed with brine (200 mL) and dried over anhydrous sodium sulfate, filtered and the filtrate was concentrated in vacuo to give the crude residue. The residue was purified by column chromatography (SiO₂. Dichloromethane:Ethyl acetate=1/0 to 20/1) to give the title compound (280 mg, 57% yield) as a yellow solid. LC-MS (ESI⁺) m/z 463.8 (M+H)⁺.

### Step 2—4-[4-(4-Bromo-5-chloro-2-fluoro-phenyl)piperazin-1-yl]-5-fluoro-2-methoxy-aniline

To a solution of 1-(4-bromo-5-chloro-2-fluoro-phenyl)-4-(2-fluoro-5-methoxy-4-nitro-phenyl) piperazine (260 mg, 562 μmol) in EtOH (10 mL) and water (5 mL) was added Fe (157 mg, 2.81 mmol) and NH₄Cl (301 mg, 5.62 mmol). Then the mixture was stirred at 80° C. for 12 hrs. On completion, the mixture was concentrated in vacuo. The residue was purified by column chromatography (SiO₂, Petroleum ether:Ethyl acetate=1/0 to 10/1) to give the title compound (246 mg) as a yellow solid. LC-MS (ESI⁺) m/z 432.8 (M+H)⁺.

### 3-[4-[4-(4-Bromo-5-chloro-2-fluoro-phenyl) piperazin-1-yl]-5-fluoro-2-methoxy-anilino]piperidine-2,6-dione (Intermediate FE)

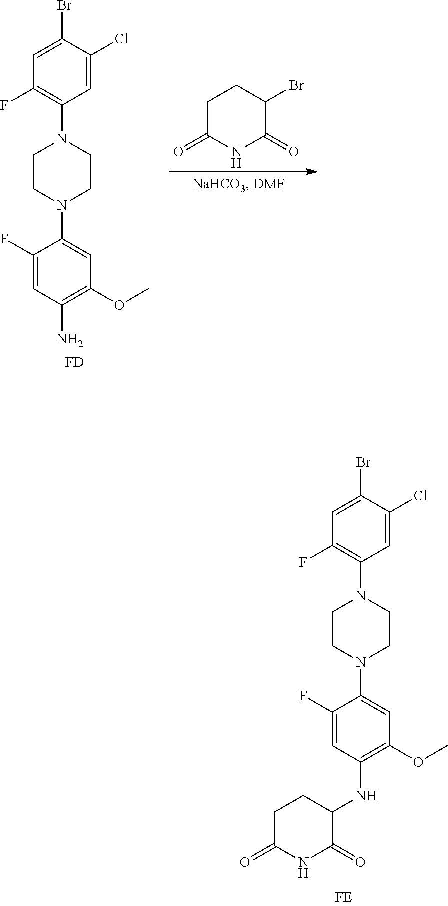

To a solution of 4-[4-(4-bromo-5-chloro-2-fluoro-phenyl)piperazin-1-yl]-5-fluoro-2-methoxy-aniline (116 mg, 268 μmol, Intermediate FD) in DMF (2 mL) was added NaHCO₃ (113 mg, 1.34 mmol, 52.2 μL) and 3-bromopiperidine-2,6-dione (103 mg, 536 μmol, CAS #62585-74-8). The mixture was then stirred at 80° C. for 12 hrs. On completion, the reaction mixture was diluted with water (20 mL) and extracted with ethyl acetate (50×2 mL). The combined organic layers were washed with brine (20 mL) and dried over anhydrous sodium sulfate, filtered and the filtrate was concentrated in vacuo to give the crude residue. The residue was purified by column chromatography (SiO₂, Petroleum ether:Ethyl acetate=1/0 to 3/1) to give the title compound (68 mg, 41% yield) as a gray solid. LC-MS (ESI⁺) m/z 545.1 (M+H)⁺.

### 1-(4-Bromo-2-fluorophenyl)piperazine (Intermediate FF)

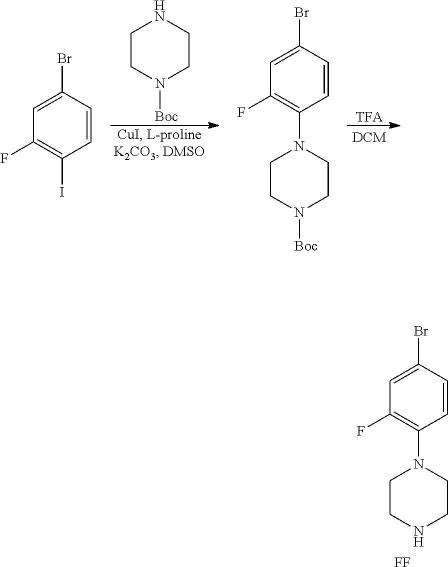

### Step 1—Tert-butyl 4-(4-bromo-2-fluorophenyl)piperazine-1-carboxylate

To a solution of 4-bromo-2-fluoro-1-iodobenzene (5 g, 16.6 mmol, CAS #105931-73-5) in DMSO (60 mL) was added tert-butyl piperazine-1-carboxylate (3.09 g, 16.6 mmol, CAS #57260-71-6), K₂CO₃ (4.59 g, 33.2 mmol), CuI (632 mg, 3.32 mmol) and L-proline (382 mg, 3.32 mmol). The mixture was degassed and purged with N₂ three times. Then the mixture was stirred at 80° C. for 12 hrs under N₂ atmosphere. On completion, the reaction mixture was diluted with water (150 mL) and extracted with ethyl acetate (3×200 mL). The combined organic layers were dried over anhydrous sodium sulfate, filtered and the filtrate was concentrated in vacuo to give the crude residue. The residue was purified by column chromatography (SiO₂, Petroleum ether/Ethyl acetate=20/1 to 10/1) to give the title compound (500 mg, 10% yield) as a gray solid. LC-MS (ESI⁺) m/z 304.9 (M−55)⁺.

### Step 2—1-(4-Bromo-2-fluorophenyl)piperazine

To a solution of tert-butyl 4-(4-bromo-2-fluorophenyl)piperazine-1-carboxylate (500 mg, 1.39 mmol) in DCM (5 mL) was added TFA (2.09 g, 18.3 mmol). The mixture was then stirred at 25° C. for 1 hr. On completion, the reaction mixture was concentrated under reduced pressure to give the title compound (1.6 g, TFA) as a brown gum, LC-MS (ESF) m/z 259.0 (M+H)⁺.

### 4-(4-(4-Bromo-2-fluorophenyl)piperazin-1-yl)-5-fluoro-2-methoxyaniline (Intermediate FG)

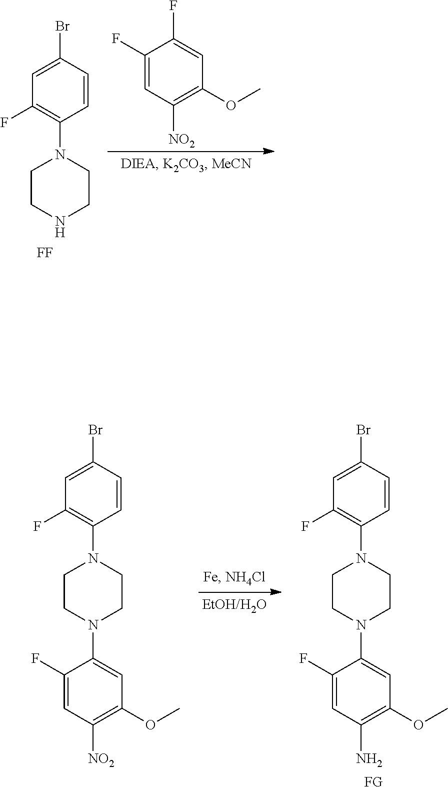

### Step 1—1-(4-Bromo-2-fluorophenyl)-4-(2-fluoro-5-methoxy-4-nitrophenyl)piperazine

To a solution of 1-(4-bromo-2-fluorophenyl)piperazine (500 mg, 1.34 mmol, TFA, Intermediate FF), 1,2-difluoro-4-methoxy-5-nitrobenzene (253 mg, 1.34 mmol, CAS #66684-64-8) in ACN (10 mL) was added, DIEA (865 mg, 6.70 mmol) and K₂CO₃ (555 mg, 4.02 mmol). The mixture was then stirred at 80° C. for 12 hrs. On completion, the reaction mixture was concentrated under reduced pressure to give a residue. The residue was purified by column chromatography (SiO₂, Petroleum ether/Ethyl acetate=10/1 to 0/1, DCM:EA=1:1) to give the title compound (500 mg, 78% yield) as a white solid. LC-MS (ESI⁺) m/z 430.1 (M+H)⁺.

### Step 2—4-(4-(4-Bromo-2-fluorophenyl)piperazin-1-yl)-5-fluoro-2-methoxyaniline

To a solution of (450 mg, 1.05 mmol) in H₂O (5 mL), EtOH (5 mL) was added Fe (293 mg, 5.25 mmol) and NH₄Cl (449 mg, 8.41 mmol). The mixture was then stirred at 80° C. for 12 hrs. On completion, the reaction mixture was filtered and the filtrate was diluted with water (10 mL) and extracted with dichloromethane (3×30 mL). The combined organic layers were dried over anhydrous sodium sulfate, filtered and the filtrate was concentrated in vacuo to give the crude residue. The residue was purified by column chromatography (SiO₂, Petroleum ether/Ethyl acetate=10/1 to 3/1) to give the title compound (400 mg, 90% yield) as a gray solid. LC-MS (ESI⁺) m/z 397.1 (M+H).

### 3-((4-(4-(4-Bromo-2-fluorophenyl)piperazin-1-yl)-5-fluoro-2-methoxyphenyl)amino)piperidine-2,6-dione (Intermediate FH)

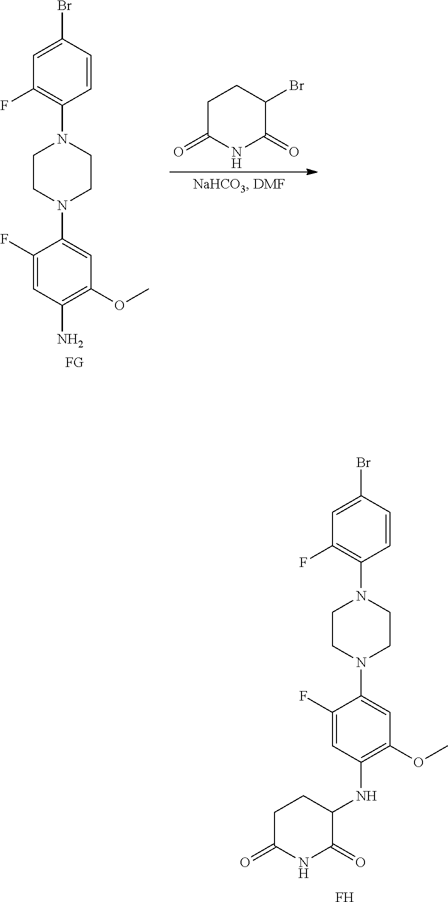

To a solution of 4-(4-(4-bromo-2-fluorophenyl)piperazin-1-yl)-5-fluoro-2-methoxyaniline (350 mg, 878 μmol, Intermediate FG), 3-bromopiperidine-2,6-dione (421 mg, 2.20 mmol, CAS #62595-74-8) in DMF (5 mL) was added NaHCO₃ (147 mg, 1.76 mmol). The mixture was then stirred at 80° C. for 12 hrs. On completion, the reaction mixture was added NH₄Cl, then extracted with ethyl acetate (3×30 mL). The combined organic layers were dried over anhydrous sodium sulfate, filtered and the filtrate was concentrated in vacuo to give the crude residue. The residue was purified by column chromatography (SiO₂, Petroleum ether/Ethyl acetate=3/1 to 0/1) to give the title compound (250 mg, 52% yield) as a brown solid. LC-MS (ESI⁺) m/z 508.8 (M+H)⁺.

### 1-(4-Bromo-2,3-difluorophenyl)piperazine (Intermediate FI)

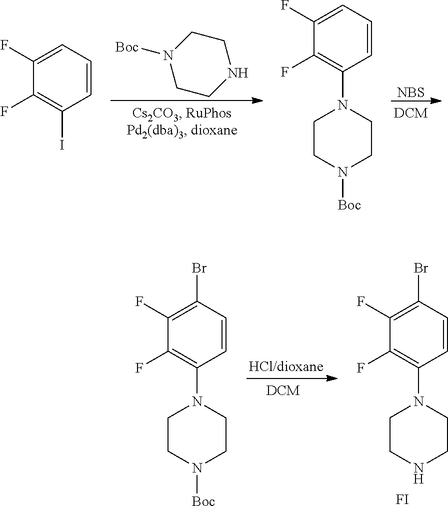

### Step 1—Tert-butyl 4-(2,3-difluorophenyl)piperazine-1-carboxylate

To a solution of 1,2-difluoro-3-iodobenzene (2 g, 8.33 mmol, CAS #64248-57-3) and tert-butyl piperazine-1-carboxylate (2.33 g, 12.5 mmol, CAS #57260-71-2) in dioxane (20 mL) was added Cs₂CO₃ (8.15 g. 25.0 mmol), RuPhos (778 mg, 1.67 mmol) and Pd₂(dba)₃ (763 mg, 833 μmol) at 25° C. Then the mixture was stirred at 100° C. for 12 hrs. On completion, the reaction mixture was concentrated in vacuo to give the crude residue. The residue was purified by column chromatography (SiO₂, Petroleum ether/Ethyl acetate=1/0 to 10/1) to give the title compound (2.3 g, 59% yield) as a yellow solid. LC-MS (ESI⁺) m/z 243.1 (M−55)⁺.

### Step 2—Tert-butyl 4-(4-bromo-2,3-difluorophenyl)piperazine-1-carboxylate

To a solution of tert-butyl 4-(2,3-difluorophenyl)piperazine-1-carboxylate (2.1 g, 7.04 mmol) in DCM (20 mL) was added NBS (1.25 g, 7.04 mmol) at 0° C., then the mixture was stirred at 25° C. for 1.5 hrs. On completion, the reaction mixture was concentrated in vacuo to give the crude residue. The residue was purified by column chromatography (SiO₂, Petroleum ether/Ethyl acetate=0/1 to 20/1) to give the title compound (2.2 g, 78% yield) as a yellow solid. LC-MS (ESI⁺) m/z 320.8 (M−55)⁺.

### Step 3—1-(4-Bromo-2,3-difluorophenyl)piperazine

To a solution of tert-butyl 4-(4-bromo-2,3-difluorophenyl)piperazine-1-carboxylate (2.08 g, 5.51 mmol) in DCM (20 mL) was added HCl/dioxane (2 M, 20 mL) at 25° C., then the mixture was stirred at 25° C. for 2 hrs. On completion, the reaction mixture was concentrated in vacuo to give the title compound (1.7 g, HCl) as a yellow solid. LC-MS (ESI⁺) m/z 276.9 (M+H)⁺.

### 4-(4-(4-Bromo-2,3-difluorophenyl)piperazin-1-yl)-5-fluoro-2-methoxyaniline (Intermediate FJ)

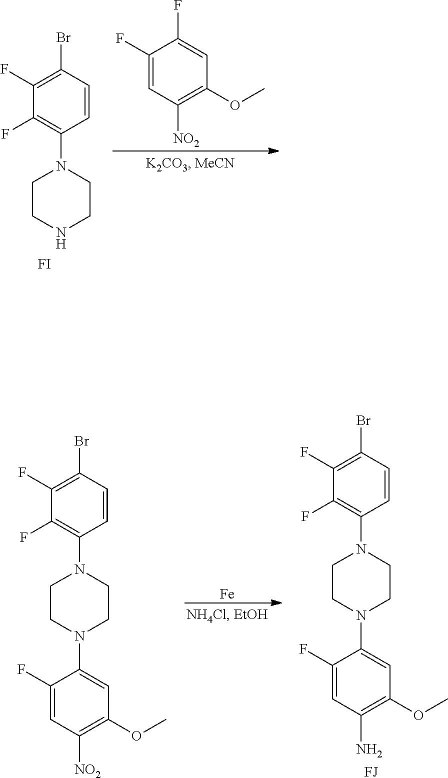

### Step 1—1-(4-Bromo-2,3-difluorophenyl)-4-(2-fluoro-5-methoxy-4-nitrophenyl)piperazine

To a solution of 1-(4-bromo-2,3-difluorophenyl)piperazine (1.2 g, 3.83 mmol, HCl, Intermediate FI) and 1,2-difluoro-4-methoxy-5-nitrobenzene (724 mg, 3.83 mmol, synthesized via Step 1 of Intermediate AQ) in ACN (10 mL) was added K₂CO₃ (2.64 g, 19.1 mmol) at 25° C. Then the mixture was stirred at 80° C. for 5 hrs. On completion, the reaction mixture was filtered to give the filtered cake as the crude residue. The residue was washed with H₂O and dried to give the title compound (1 g, 50% yield) as a yellow solid. LC-MS (ESI⁺) m/z 448.0 (M+H)⁺.

### Step 2—4-(4-(4-Bromo-2,3-difluorophenyl)piperazin-1-yl)-5-fluoro-2-methoxyaniline

To a solution of 1-(4-bromo-2,3-difluorophenyl)-4-(2-fluoro-5-methoxy-4-nitrophenyl)piperazine (900 mg. 2.02 mmol) in EtOH (6 mL) and H₂O (6 mL) was added Fe (676 mg, 12.1 mmol) and NH₄Cl (647 mg, 12.1 mmol) at 25° C. Then the mixture was stirred at 80° C. for 1 hr. On completion, the reaction mixture was filtered and concentrated in vacuo to give the title compound (220 mg) as a yellow solid. LC-MS (ESI⁺) m/z 416.2 (M+H)⁺.

### 3-((4-(4-(4-Bromo-2,3-difluorophenyl)piperazin-1-yl)-5-fluoro-2-methoxyphenyl)amino)piperidine-2,6-dione (Intermediate FK)

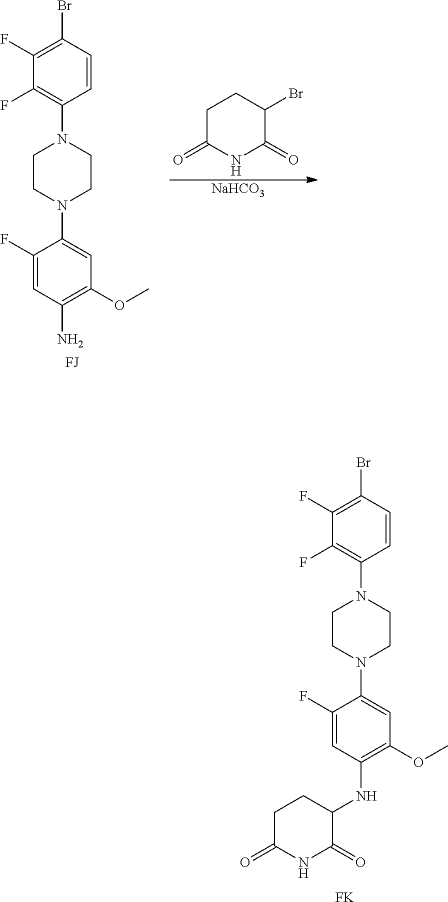

To a solution of 4-(4-(4-bromo-2,3-difluorophenyl)piperazin-1-yl)-5-fluoro-2-methoxyaniline (220 mg, 529 μmol, Intermediate FJ) and 3-bromopiperidine-2,6-dione (122 mg, 634.26 μmol, CAS #62595-74-8) in DMF (2 mL) was added NaHCO₃ (88.8 mg, 1.06 mmol) at 25° C. Then the mixture was stirred at 80° C. for 12 hrs. On completion, the reaction mixture was quenched with water (5 mL) and extracted with ethyl acetate (5×3 mL). The extracts were washed with brine (100 mL) and dried over anhydrous sodium sulfate, filtered and concentrated in vacuo to give the crude residue. The residue was purified by column chromatography (SiO₂, Petroleum ether/Ethyl acetate=10/1 to 5/1) to give the title compound (120 mg, 43% yield) as a yellow solid. LC-MS (ESI⁺) m/z 526.9 (M+H)⁺.

### 3-(4-(4-(4-Chloro-3-methylphenyl)piperidin-1-yl)-3-fluorophenyl)piperidine-2,6-dione (Intermediate FL)

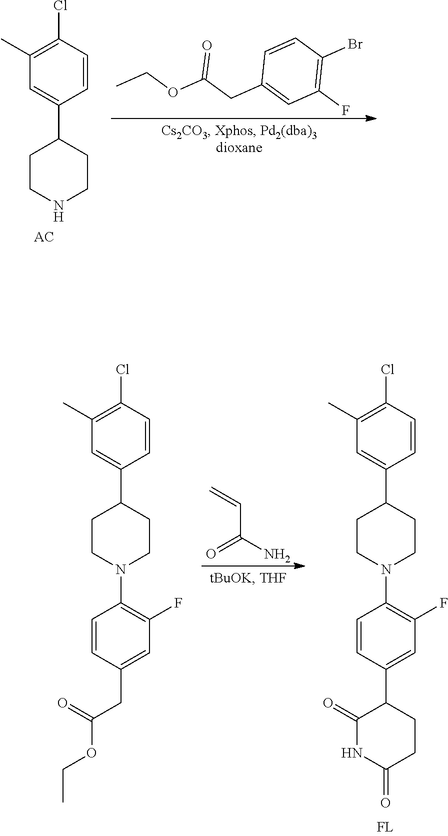

### Step 1—Ethyl 2-(4-(4-(4-chloro-3-methylphenyl)piperidin-1-yl)-3-fluorophenyl)acetate

A mixture of 4-(4-chloro-3-methylphenyl)piperidine (2.1 g, 6.49 mmol, TFA, Intermediate AC), ethyl 2-(4-bromo-3-fluorophenyl)acetate (2.54 g, 9.73 mmol, CAS #1296223-82-9), Pd₂(dba)₃ (297 mg, 324 μmol), XPhos (371 mg, 778 μmol) and Cs₂CO₃ (8.45 g, 26.0 mmol) in toluene (20 mL) was degassed and purged with N₂ three times. Then the mixture was stirred at 100° C. for 2 hrs under N₂ atmosphere. On completion, the reaction mixture was quenched with water (30 mL) and extracted with DCM (3×30 mL). The combined organic layers were washed with aqueous NaCl (30 mL) and dried over anhydrous sodium sulfate, filtered and the filtrate was concentrated in vacuo to give the crude residue. The residue was purified by column chromatography (SiO₂, Petroleum ether:Ethyl acetate=49/1) to give the title compound (1.4 g, 44% yield) as a yellow solid. LC-MS (ESI⁺) m/z 389.9 (M+H)⁺.

### Step 2—3-(4-(4-(4-chloro-3-methylphenyl)piperidin-1-yl)-3-fluorophenyl)piperidine-2,6-dione

A mixture of ethyl 2-(4-(4-(4-chloro-3-methylphenyl)piperidin-1-yl)-3-fluorophenyl)acetate (700 mg, 1.53 mmol), acrylamide (108 mg, 1.53 mmol, CAS #9003-05-8), tBuOK (1 M, 1.53 mL) in THF (3 mL) was degassed and purged with N₂ three times. Then the mixture was stirred at 40° C. for 1 hr under N₂ atmosphere. On completion, the reaction mixture was quenched with water (30 mL) and extracted with EA (3×30 mL). The combined organic layers were washed with aqueous NaCl (30 mL) and dried over anhydrous sodium sulfate, filtered and the filtrate was concentrated in vacuo to give the crude residue. The residue was purified by column chromatography (SiO₂, Petroleum ether:Ethyl acetate=4/1) to give the title compound (144 mg, 23% yield) as a yellow solid. LC-MS (ESI⁺) m/z 415.1 (M+H)⁺.

### 4-(4-Chloro-3-fluorophenyl)piperidine (Intermediate FM)

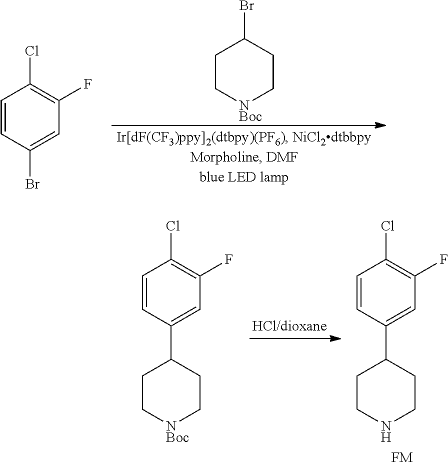

### Step 1—Tert-butyl 4-(4-chloro-3-fluorophenyl)piperidine-1-carboxylate

To a solution of tert-butyl 4-bromopiperidine-1-carboxylate (32.8 g, 124 mmol, CAS #180695-79-8) and 1-bromo-4-chloro-2-fluoro-benzene (20.0 g. 95.4 mmol, CAS #60811-18-9) in DMF (300 mL) and was added 2,2,3,3,5,5,6,6-octadeuteriomorpholine (12.5 g. 143 mmol), bis[3,5-difluoro-2-[5-(trifluoromethyl)-2-pyridyl]phenyl]iridium (1+); 4-tert-butyl-2-(4-tert-butyl-2-pyridyl)pyridine; hexafluorophosphate (1.07 g, 955 μmol) and 4-tert-butyl-2-(4-tert-butyl-2-pyridyl)pyridine dichloronickel (1.90 g, 4.77 mmol). Then the mixture was stirred and irradiated with a 455 nm LED lamp (3 cm away) by flow reaction, with cooling water to keep the reaction temperature at 25° C. for 12 hrs. On completion, the crude product was purified by reverse phase HPLC (0.1% FA condition). Then the product was extracted with EA (500 mL×2). The combined organic layers were washed with NaCl 1500 mL (500 mL×3), dried over Na₂SO₄, filtered and concentrated under reduced pressure to give the title compound (11.7 g, 38% yield) as a yellow oil. LC-MS (ESI⁺) m/z 258.2 (M−55)⁺; ¹H NMR (400 MHz, CDCl₃-d) 6=7.31 (t, J=8.0 Hz, 1H), 6.98 (d, J=10.4 Hz, 1H), 6.93 (d, J=8.4 Hz, 1H), 4.24 (s, 2H), 2.79 (t, J=12.0 Hz, 2H), 2.68-2.59 (m, 1H), 1.81 (d, J=12.8 Hz, 2H), 1.62-1.54 (m, 2H), 1.48 (s, 9H).

### Step 2—4-(4-Chloro-3-fluorophenyl)piperidine

To a solution of tert-butyl 4-(4-chloro-3-fluoro-phenyl)piperidine-1-carboxylate (11 g, 35 mmol) in DCM (80.0 mL) was added HCl/dioxane (6 M, 40.0 mL). The mixture was then stirred at 25° C. for 1 hr. On completion, the mixture was concentrated under reduce pressure to give a residue, then diluted with THF (100 mL) and NH₃H₂O (40 mL). After stirring for 30 minutes, the solution was extracted with EA (200 mL×1). The combined organic layers were concentrated by freeze drying to give the title compound (7.00 g, 90% yield) as a yellow oil. LC-MS (ESI⁺) m/z 213.8 (M+H)⁺.

### 3-(4-Bromo-3-fluorophenyl)-1-(4-methoxybenzyl)piperidine-2,6-dione (Intermediate FN)

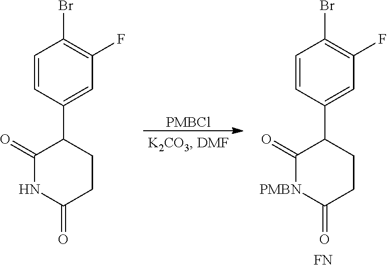

To a solution of 3-(4-bromo-3-fluorophenyl)piperidine-2,6-dione (1 g, 3.50 mmol, synthesized via Step 1 of Intermediate B) in DMF (20 mL) was added K₂CO₃ (1.45 g, 10.5 mmol) and 1-(chloromethyl)-4-methoxy-benzene (602 mg, 3.84 mmol, 522 μL). The mixture was then stirred at 40° C. for 12 hr under N₂ atmosphere. On completion, the reaction mixture was diluted with H₂O (50 mL) and extracted with EA (30 mL×3). The combined organic layers were washed with aqueous NaCl (40 mL×2), dried over Na₂SO₄, filtered and concentrated under reduced pressure to give a residue. The residue was purified by column chromatography (SiO₂, Dichloromethane/Ethyl acetate=1/0 to 10/1) to give the title compound (1.2 g, 68% yield) as a yellow oil. LC-MS (ESI⁺) m/z 406.0 (M+H)⁺.

### 3-(4-(4-(4-Chloro-3-fluorophenyl)piperidin-1-yl)-3-fluorophenyl)piperidine-2,6-dione (Intermediate FO)

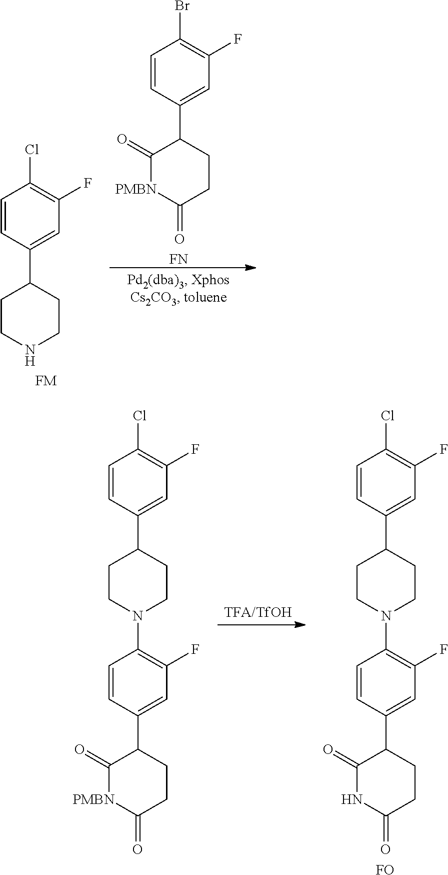

### Step 1—3-(4-(4-(4-Chloro-3-fluorophenyl)piperidin-1-yl)-3-fluorophenyl)-1-(4-methoxybenzyl)piperidine-2,6-dione

A mixture of 4-(4-chloro-3-fluoro-phenyl)piperidine (526 mg, 2.46 mmol, Intermediate FM), 3-(4-bromo-3-fluorophenyl)-1-(4-methoxybenzyl)piperidine-2,6-dione (1.00 g, 2.46 mmol, Intermediate FN). Pd₂(dba)₃ (113 mg, 123 μmol), XPhos (117 mg, 246 μmol) and Cs₂CO₃ (2.41 g, 7.38 mmol) in toluene (12.0 mL) was degassed and purged with N₂ three times. Then the mixture was stirred at 100° C. for 1 hr under N₂ atmosphere. On completion, the reaction mixture was filtered and concentrated under reduced pressure to give a residue. The mixture was extracted with EA (20 mL×2). The combined organic layers were washed with NaCl (20 mL×3), dried over Na₂SO₄, filtered and concentrated under reduced pressure to give a residue. The crude product was purified by MPLC(SiO₂, PE:EA=10:1 to 5:1) to give the title compound (770 mg, 34% yield) was obtained as a yellow solid. LC-MS (ESI⁺) m/z 539.0 (M+H)⁺.

### Step 2—3-(4-(4-(4-Chloro-3-fluorophenyl)piperidin-1-yl)-3-fluorophenyl)piperidine-2,6-dione

To a solution of 3-[4-[4-(4-chloro-3-fluoro-phenyl)-1-piperidyl]-3-fluoro-phenyl]-1-[(4-methoxyphenyl)methyl]piperidine-2,6-dione (720 mg, 788 μmol) in TFA (7.20 mL) and was added TfOH (2.40 mL), then the mixture was stirred at 70° C. for 1 hr. On completion, the reaction mixture was concentrated under reduced pressure. The crude product was purified by reverse phase HPLC (0.1% FA condition) to give the title compound (160 mg, 40% yield, FA) as a yellow solid. LC-MS (ESI⁺) m/z 419.0 (M+H)⁺.

### 2-Methyl-3-(1H-pyrazol-1-yl)-1-(5-(4,4,5,5-tetramethyl-1,3,2-dioxaborolan-2-yl)-3,6-dihydropyridin-1 (2H)-yl)propan-1-one (Intermediate FP)

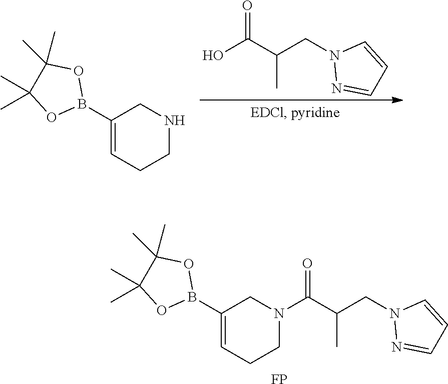

To a solution of 5-(4,4,5,5-tetramethyl-1,3,2-dioxaborolan-2-yl)-1,2,3,6-tetrahydropyridine (2.03 g, 9.73 mmol, synthesized via Step 1 of Intermediate E) in pyridine (10 mL) was added EDCI (2.80 g 14.5 mmol). Then 2-methyl-3-pyrazol-1-yl-propanoic acid (1.5 g, 9.73 mmol, CAS #197094-12-5) was added and the mixture was stirred at 20° C. for 2 hrs. On completion, the reaction mixture was concentrated under reduced pressure to remove solvent. The residue was purified by column chromatography (SiO₂, Petroleum ether/Ethyl acetate=5/1 to 1/1) to give the title compound (1.4 g, 40% yield) as a gray solid. LC-MS (ESI⁺) m/z 345.9 (M+H)⁺.

### (S)-4-chloro-7-fluoro-N,N-dimethyl-6-(1-(2-methyl-3-(1H-pyrazol-1-yl)propanoyl)-1,2,5,6-tetrahydropyridin-3-yl)-1H-indole-2-carboxamide (Intermediate FQ) and (R)-4-chloro-7-fluoro-N,N-dimethyl-6-(1-(2-methyl-3-(1H-pyrazol-1-yl)propanoyl)-1,2,5,6-tetrahydropyridin-3-yl)-1H-indole-2-carboxamide (Intermediate FR)

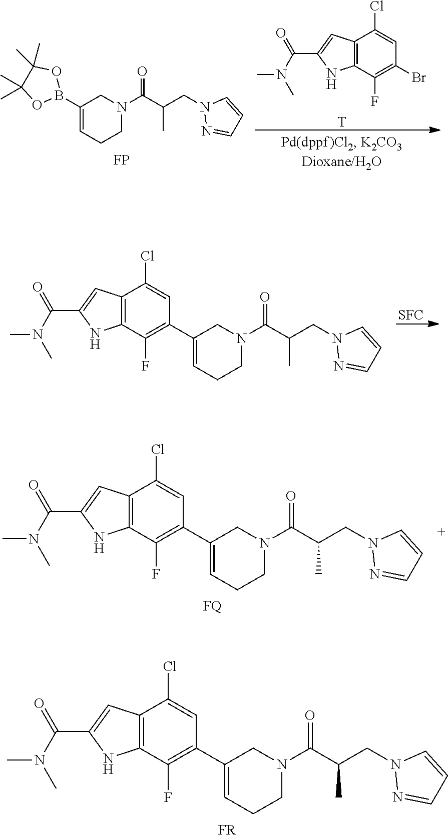

### Step 1—4-Chloro-7-fluoro-N,N-dimethyl-6-(1-(2-methyl-3-(1H-pyrazol-1-yl)propanoyl)-1,2,5,6-tetrahydropyridin-3-yl)-1H-indole-2-carboxamide

A mixture of 2-methyl-3-pyrazol-1-yl-1-[5-(4,4,5,5-tetramethyl-1,3,2-dioxaborolan-2-yl)-3,6-dihydro-2H-pyridin-1-yl]propan-1-one (1.3 g, 3.77 mmol, Intermediate FP). 6-bromo-4-chloro-7-fluoro-N,N-dimethyl-1H-indole-2-carboxamide (1.20 g, 3.77 mmol, Intermediate T), Pd(dppf)Cl₂ (275 mg, 376 μmol), and K₂CO₃ (1.56 g. 11.3 mmol) in dioxane (2 mL), H₂O (0.5 mL) was degassed and purged with N₂ three times. Then the mixture was stirred at 80° C. for 2 hrs under N₂ atmosphere. On completion, the reaction mixture was concentrated under reduced pressure to give a residue. The residue was purified by column chromatography (SiO₂, Petroleum ether/Ethyl acetate=3/1 to 1/1, DCM:EA=1:1) to give the title compound (1.3 g, 75% yield) as a yellow solid. LC-MS (ESI⁺) m/z 458.1 (M+H)⁺.

### Step 2—(S)-4-chloro-7-fluoro-N,N-dimethyl-6-(1-(2-methyl-3-(1H-pyrazol-1-yl)propanoyl)-1,2,5,6-tetrahydropyridin-3-yl)-1H-indole-2-carboxamide and (R)-4-chloro-7-fluoro-N,N-dimethyl-6-(1-(2-methyl-3-(1H-pyrazol-1-yl)propanoyl)-1,2,5,6-tetrahydropyridin-3-yl)-1H-indole-2-carboxamide

To a solution of 4-chloro-7-fluoro-N,N-dimethyl-6-(1-(2-methyl-3-(1H-pyrazol-1-yl)propanoyl)-1,2,5,6-tetrahydropyridin-3-yl)-1H-indole-2-carboxamide (1.3 g,) was purified by column chromatography (column: Daicel chiralpak IK (250 mm×30 mm, 10 um); mobile phase: [CO₂-i-PrOH (0.1% NH₃H₂O)]; B %: 40%, isocratic elution mode) to give (S)-4-chloro-7-fluoro-N,N-dimethyl-6-(1-(2-methyl-3-(1H-pyrazol-1-yl)propanoyl)-1,2,5,6-tetrahydropyridin-3-yl)-1H-indole-2-carboxamide (590 mg, 45% yield) as a white solid, and (R)-4-chloro-7-fluoro-N,N-dimethyl-6-(1-(2-methyl-3-(1H-pyrazol-1-yl)propanoyl)-1,2,5,6-tetrahydropyridin-3-yl)-1H-indole-2-carboxamide (550 mg, 42% yield) as a white solid. LC-MS (ESI⁺) m/z 458.1 (M+H)⁺ for both isomers. The absolute stereochemistry of the enantiomers was assigned arbitrarily.

### (S)-7-fluoro-N,N-dimethyl-6-(1-(2-methyl-3-(1H-pyrazol-1-yl)propanoyl)-1,2,5,6-tetrahydropyridin-3-yl)-4-(4,4,5,5-tetramethyl-1,3,2-dioxaborolan-2-yl)-1H-indole-2-carboxamide (Intermediate FS)

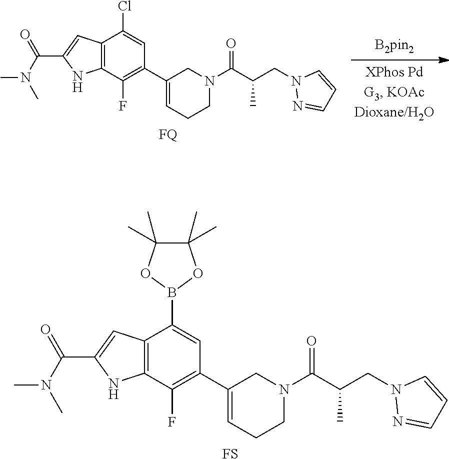

A mixture of 4-chloro-7-fluoro-N,N-dimethyl-6-[1-[(2S)-2-methyl-3-pyrazol-1-yl-propanoyl]-3,6-dihydro-2H-pyridin-5-yl]-1H-indole-2-carboxamide (250 mg, 545 μmol, Intermediate FQ), 4,4,5,5-tetramethyl-2-(4,4,5,5-tetramethyl-1,3,2-dioxaborolan-2-yl)-1,3,2-dioxaborolane (346 mg, 1.36 mmol, CAS #73183-34-3), KOAc (160 mg, 1.64 mmol), and XPhos Pd G3 (46.2 mg, 54.5 μmol) in dioxane (2 mL) was degassed and purged with N₂ three times. Then the mixture was stirred at 80° C. for 2 hrs under N₂ atmosphere. On completion, the reaction mixture was concentrated under reduced pressure to remove solvent. The residue was purified by column chromatography (SiO₂, Petroleum ether/Ethyl acetate=3/1 to 0/1) to give the title compound (250 mg, 65% yield) as a gray solid. LC-MS (ESI⁺) m/z 550.2 (M+H)⁻.

### 4-(4-(4-Bromophenyl)piperidin-1-yl)-3-fluoroaniline (Intermediate FT)

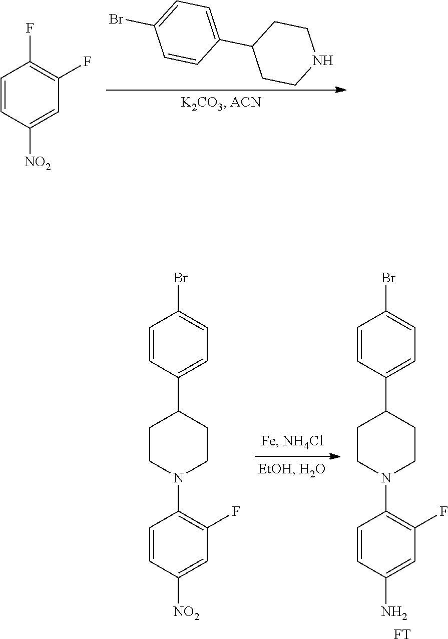

### Step 1—4-(4-bromophenyl)-1-(2-fluoro-4-nitrophenyl)piperidine

To a solution of 1,2-difluoro-4-nitro-benzene (50 g, 314 mmol, 34.8 mL, CAS #369-34-6) and 4-(4-bromophenyl)piperidine (83.0 g, 346 mmol, CAS #80980-89-8) in ACN (800 mL) was added K₂CO₃ (86.8 g, 628 mmol). The mixture was then stirred at 80° C. for 2 hr. On completion, the mixture was filtered then the filtrated was concentrated by vacuum distillation to obtain the crude product. The residue was purified by column chromatography (SiO₂, Petroleum ether:Ethyl acetate=100/1 to 10/1) to give the title compound (60 g, 50% yield) as a yellow solid. ¹H NMR (400 MHz, DMSO-d₆, 300 K) S (ppm)=8.03-8.01 (m, 1H), 8.01-7.98 (m, 1H), 7.52-7.49 (m, 1H), 7.49-7.47 (m, 1H), 7.28-7.26 (m, 1H), 7.25-7.24 (s, 1H), 7.23-7.19 (m, 1H), 3.83-3.78 (d, J=12.8 Hz, 2H), 3.08-3.01 (m, 2H), 2.82-2.76 (m, 1H), 1.90-1.85 (m, 2H), 1.80-1.72 (m, 2H).

### Step 2—4-(4-(4-Bromophenyl)piperidin-1-yl)-3-fluoroaniline

To a solution of 4-(4-bromophenyl)-1-(2-fluoro-4-nitro-phenyl)piperidine (59 g, 155 mmol) in H₂O (500 mL) and EtOH (500 mL) was added Fe (60.8 g, 1.09 mol) and NH₄Cl (58.2 g, 1.09 mol). The mixture was then stirred at 80° C. for 3 h. On completion, the reaction mixture was concentrated under reduced pressure to remove solvent. The residue was diluted with EA (800 mL) and extracted with EA (500 mL×3). The combined organic layer was dried by Na₂SO₄, filtered, and reduced pressure concentrated to obtain the title compound (37 g) as a brown solid. ¹H NMR (400 MHz, DMSO-d₆, 299 K) δ (ppm)=7.53-7.41 (d, J=8.0 Hz, 2H), 7.29-7.20 (d, J=8.0 Hz, 2H), 6.85-6.75 (t, J=9.2 Hz, 1H), 6.41-6.28 (m, 2H), 5.13-4.88 (s, 2H), 3.23-3.12 (d, J=11.2 Hz, 2H), 2.69-2.60 (m, 2H), 2.59-2.53 (m, 1H), 1.82-1.70 (m, 4H).

### 3-((4-(4-(4-Bromophenyl)piperidin-1-yl)-3-fluorophenyl)amino)piperidine-2,6-dione (Intermediate FU)

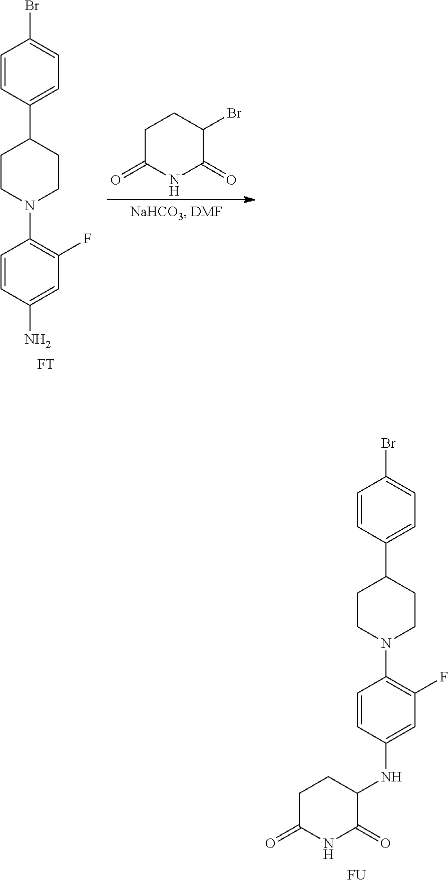

To a solution of 4-[4-(4-bromophenyl)-1-piperidyl]-3-fluoro-aniline (35 g, 100 mmol, Intermediate FT) and 3-bromopiperidine-2,6-dione (38.5 g, 200 mmol) in DMF (500 mL) was added NaHCO₃ (16.8 g, 200 mmol, 7.80 mL). The mixture was then stirred at 90° C. for 12 hr. On completion, reaction was filtered and the residue was diluted with H₂O (1000 mL) and saturated NaCl solution (500 mL×3), then extracted with EA (500 mL×3). The combined with the organic layer, dried, filtered and concentrated with Na₂SO₄ to obtain a residue. The residue was purified by reverse phase HPLC (0.1% FA condition) to give the title compound (32 g. 69% yield) as a white solid. ¹H NMR (400 MHz, DMSO-d₆, 299 K) δ (ppm)=10.81-10.73 (s, 1H), 7.52-7.45 (d, J=8.4 Hz, 2H), 7.28-7.24 (d, J=8.4 Hz, 2H), 6.93-6.83 (t, J=9.2 Hz, 1H), 6.57-6.49 (m, 1H), 6.46-6.41 (m, 1H), 5.96-5.68 (s, 1H), 4.30-4.22 (d, J=7.2 Hz, 1H), 3.25-3.20 (d, J=10.8 Hz, 2H), 2.79-2.67 (m, 3H), 2.62-2.54 (m, 2H), 2.13-2.04 (m, 1H), 1.89-1.82 (m, 2H), 1.81-1.74 (m, 3H).

### 3-((3-Fluoro-4-(4-(4-(4,4,5,5-tetramethyl-1,3,2-dioxaborolan-2-yl)phenyl)piperidin-1-yl)phenyl)amino)piperidine-2,6-dione (Intermediate FV)

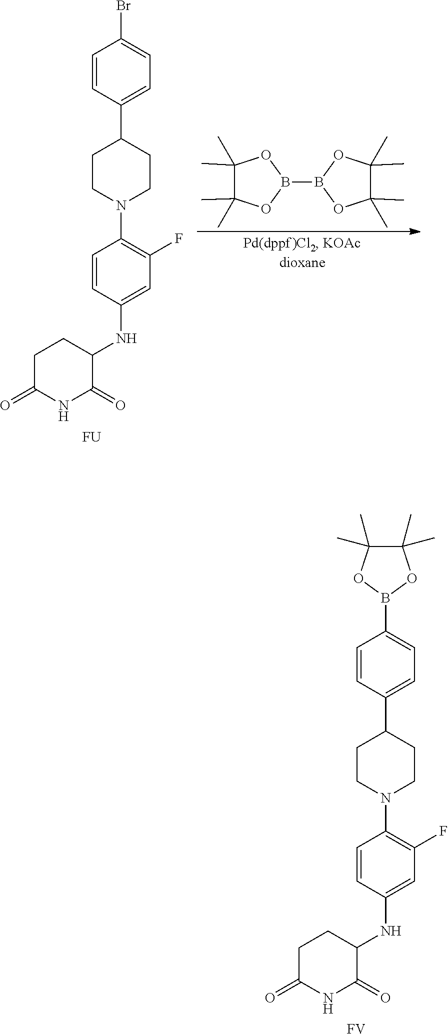

To a solution of 3-((4-(4-(4-bromophenyl)piperidin-1-yl)-3-fluorophenyl)amino)piperidine-2,6-dione (150 mg, 311 μmol, Intermediate FU), 4,4,4′,4′,5,5,5′,5′-octamethyl-2,2′-bi(1,3,2-dioxaborolane) (150 mg, 311 μmol, CAS #73183-34-3) in dioxane (10.0 mL) was added KOAc (426 mg, 4.34 mmol) and Pd(dppf)Cl₂ (158 mg, 217 μmol). Then the reaction was stirred at 90° C. for 2 hrs under nitrogen atmosphere. On completion, the reaction mixture was concentrated in vacuo to give the crude residue. The crude residue was purified by column chromatography (SiO₂, Petroleum ether/Ethyl acetate=10/1 to 1/1) to give the title compound (1.00 g. 82% yield) as a yellow solid. LC-MS (ESI⁺) m/z 508.1 (M+H)⁺.

### 3-(4-(4-(4-Chlorophenyl)piperazin-1-yl)-3-fluorophenyl)piperidine-2,6-dione (Intermediate FW)

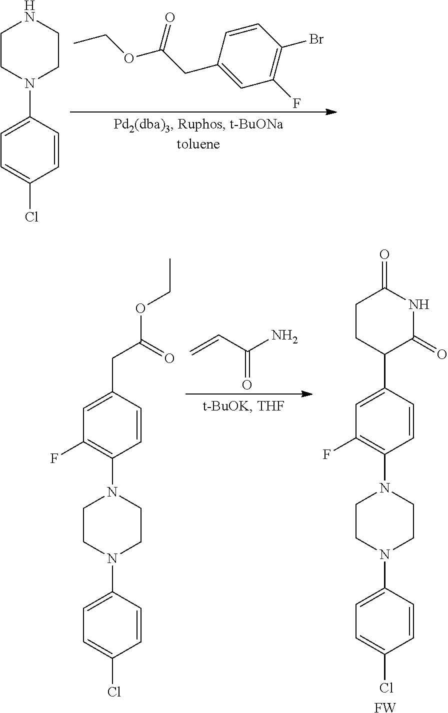

### Step 1—Ethyl 2-(4-(4-(4-chlorophenyl)piperazin-1-yl)-3-fluorophenyl)acetate

A mixture of 1-(4-chlorophenyl)piperazine (2.00 g, 10.1 mmol, CAS #38212-33-8), ethyl 2-(4-bromo-3-fluoro-phenyl)acetate (2.65 g, 10.1 mmol, CAS #1296233-82-9), Pd₂(dba)₃ (465 mg, 508 μmol), Cs₂CO₃ (9.94 g, 30.5 mmol) and XPhos (581 mg, 1.22 mmol) in toluene (40 mL) was degassed and purged with N₂ three times. Then the mixture was stirred at 100° C. for 2 h under N₂ atmosphere. On completion, the reaction mixture was filtered and concentrated under reduced pressure to give a residue. The residue was purified by flash silica gel chromatography (ISCO®; 40 g SepaFlash® Silica Flash Column, Eluent of 0˜5% Ethyl acetate/Petroleum ether gradient @80 mL/min) to afford the title compound (1.90 g, 50% yield) as a yellow solid. LC-MS (ESI⁺) m/z 377.1 (M+H)⁺; ¹H NMR (400 MHz, CDCl₃) δ ppm 7.23 (s, 2H), 7.08-6.99 (m, 2H), 6.98-6.87 (m, 3H), 4.17 (q, J=7.2 Hz, 2H), 3.56 (s, 2H), 3.36-3.29 (m, 4H), 3.27-3.15 (m, 4H), 1.27 (t, J=7.2 Hz, 3H).

### Step 2—3-(4-(4-(4-Chlorophenyl)piperazin-1-yl)-3-fluorophenyl)piperidine-2,6-dione

To a solution of ethyl 2-[4-[4-(4-chlorophenyl)piperazin-1-yl]-3-fluoro-phenyl]acetate (1.70 g, 4.51 mmol) and prop-2-enamide (288 mg, 4.06 mmol) in THF (17 mL) was added t-BuOK (1 M, 4.96 mL) at 0° C. The mixture was then stirred at 50° C. for 2 hr. On completion, the reaction mixture was quenched by addition of aqueous NH₄Cl (30 mL), and then extracted with ethyl acetate (20 mL×3). The combined organic layers were dried over Na₂SO₄, filtered and concentrated under reduced pressure to give a residue. The residue was purified by prep-HPLC (column: Waters Xbridge BEH C18 250*50 mm*10 um; mobile phase: [water (NH₄HCO₃)-ACN]; gradient: 44%-74% B over 5 min) to afford the title compound (350 mg, 19% yield) as a white solid. LC-MS (ESI⁺) m/z 402.1 (M+H)⁺; ¹H NMR (400 MHz, DMSO-d₆) δ ppm 10.82 (s, 1H), 7.26 (d, J=8.8 Hz, 2H), 7.10-6.98 (m, 5H), 3.82 (dd, J=4.8, 12.0 Hz, 1H), 3.29 (d, J=6.0 Hz, 5H), 3.19-3.10 (m, 4H), 2.67-2.60 (m, 1H), 2.28-2.15 (m, 1H), 2.07-1.96 (m, 1H).

### (E)-1-chloro-4-(2-ethoxyvinyl)-2-methoxybenzene (Intermediate FX)

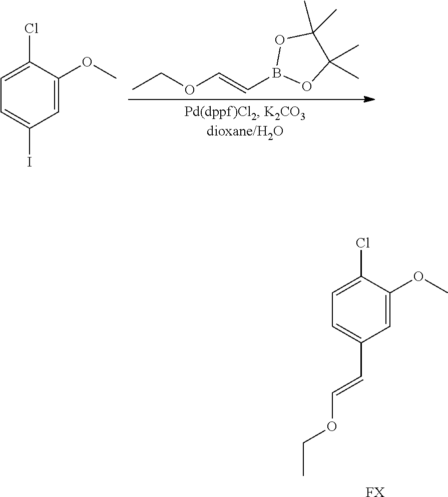

A mixture of 1-chloro-4-iodo-2-methoxy-benzene (2.3 g, 8.6 mmol, CAS #161949-50-4), 2-[(E)-2-ethoxyvinyl]-4,4,5,5-tetramethyl-1,3,2-dioxaborolane (1.87 g, 9.42 mmol, CAS #1201905-61-4), Pd(dppf)Cl₂ (627 mg, 857 μmol), and K₂CO₃ (3.55 g, 25.7 mmol) in dioxane (28 mL) and H₂O (7 mL) was degassed and purged with N₂ three times. Then the mixture was stirred at 60° C. for 2 hrs under N₂ atmosphere. On completion, the reaction mixture was concentrated under reduced pressure to give a residue. The residue was purified by column chromatography (SiO₂, Petroleum ether/Ethyl acetate=1/0 to 24/1) to give the title compound (0.9 g, 47% yield) as a yellow oil. LC-MS (ESI⁻) m/z 213.2 (M+H)⁺.

### 6-(1-(3-(1H-1,2,3-triazol-1-yl)propanoyl)-1,2,5,6-tetrahydropyridin-3-yl)-7-fluoro-4-(2-methoxy-4-(2-oxoethyl)phenyl)-N,N-dimethyl-1H-indole-2-carboxamide (Intermediate FY)

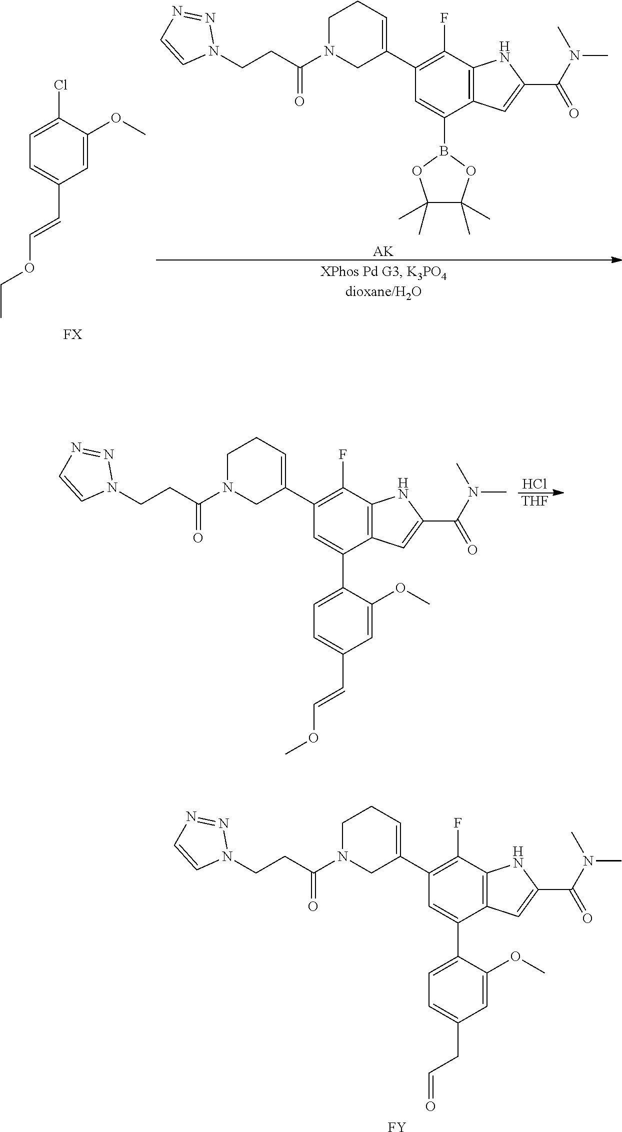

### Step 1—(E)-6-(1-(3-(1H-1,2,3-triazol-1-yl)propanoyl)-1,2,5,6-tetrahydropyridin-3-yl)-4-(4-(2-ethoxyvinyl)-2-methoxyphenyl)-7-fluoro-N,N-dimethyl-1H-indole-2-carboxamide

A mixture of 7-fluoro-N,N-dimethyl-4-(4,4,5,5-tetramethyl-1,3,2-dioxaborolan-2-yl)-6-[1-[3-(triazol-1-yl)propanoyl]-3,6-dihydro-2H-pyridin-5-yl]-1H-indole-2-carboxamide (190 mg, 354 μmol, Intermediate AK), 1-chloro-4-[(E)-2-ethoxyvinyl]-2-methoxy-benzene (82.9 mg, 390 μmol, Intermediate FX), XPhos Pd G3 (30.0 mg, 35.4 μmol), and K₃PO₄ (226 mg, 1.06 mmol) in dioxane (4 mL) and H₂O (1 mL) was degassed and purged with N₂ three times. Then the mixture was stirred at 80° C. for 2 hrs under N₂ atmosphere. On completion, the reaction mixture was concentrated under reduced pressure to give a residue. The residue was purified by column chromatography (SiO₂. Petroleum ether/Ethyl acetate=1/1 to 0/1 and then DCM/MeOH=1/0 to 10/1) to give the title compound (130 mg, 53% yield) as a yellow solid. LC-MS (ESI⁺) m/z 587.3 (M+H)⁺.

### Step 2—6-(1-(3-(TH-1,2,3-triazol-1-yl)propanoyl)-1,2,5,6-tetrahydropyridin-3-yl)-7-fluoro-4-(2-methoxy-4-(2-oxoethyl)phenyl)-N,N-dimethyl-1H-indole-2-carboxamide

To a solution of 4-[4-[(E)-2-ethoxyvinyl]-2-methoxy-phenyl]-7-fluoro-N,N-dimethyl-6-[1-[3-(triazol-1-yl)propanoyl]-3,6-dihydro-2H-pyridin-5-yl]-1H-indole-2-carboxamide (130 mg, 222 μmol) in THE (1 mL) was added HCl (3 M, 1 mL). The mixture was then stirred at 20° C. for 0.5 hrs. On completion, the reaction mixture was concentrated under reduced pressure to a residue. The residue was purified by prep-HPLC (0.1% FA condition) to the title compound (80 mg, 58% yield) as a white solid. LC-MS (ESI⁺) m/z 559.4 (M+H)⁺; ¹H NMR (400 MHz, DMSO-d₆) δ=10.80 (s, 1H), 7.03-6.99 (m, 1H), 6.98-6.93 (m, 2H), 3.79 (dd, J=4.8, 12.0 Hz, 1H), 2.93-2.87 (m, 4H), 2.85-2.80 (m, 4H), 2.70-2.58 (m, 1H), 2.46 (s, 1H), 2.19 (dq, J=4.4, 12.4 Hz, 1H), 2.02-1.97 (m, 1H).

### 3-(3-Fluoro-4-(piperazin-1-yl)phenyl)piperidine-2,6-dione (Intermediate FZ)

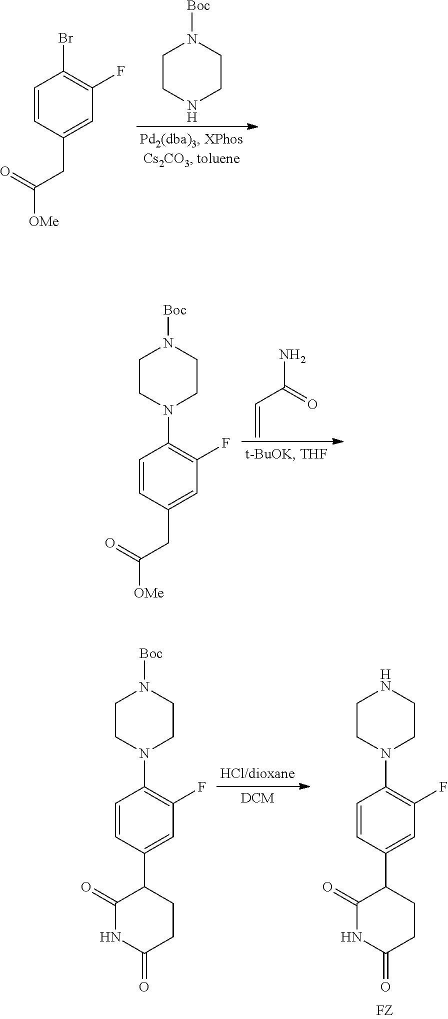

### Step 1—Tert-butyl 4-(2-fluoro-4-(2-methoxy-2-oxoethyl)phenyl)piperazine-1-carboxylate

A mixture of methyl 2-(4-bromo-3-fluoro-phenyl)acetate (5.68 g, 23.0 mmol, CAS #942282-41-9), tert-butyl piperazine-1-carboxylate; hydrochloride (7.68 g, 34.5 mmol. CAS #57260-71-6), Pd₂(dba)₃ (1.05 g, 1.15 mmol), Cs₂CO₃ (22.5 g, 68.9 mmol) and XPhos (1.31 g, 2.76 mmol) in toluene (100 mL) was degassed and purged with N₂ three times. Then the mixture was stirred at 100° C. for 2 hrs under N₂ atmosphere. On completion, the reaction mixture was filtered and the filtrate was concentrated under reduced pressure to give a residue. The residue was purified by column chromatography (SiO₂, Petroleum ether/Ethyl acetate=1/0 to 9/1) to give the title compound (8 g, 64% yield) as a yellow oil. LC-MS (ESI⁺) m/z 353.0 (M+H)⁺.

### Step 2—Tert-butyl 4-(4-(2,6-dioxopiperidin-3-yl)-2-fluorophenyl)piperazine-1-carboxylate

To a solution of tert-butyl 4-[2-fluoro-4-(2-methoxy-2-oxo-ethyl)phenyl]piperazine-1-carboxylate (2 g, 6 mmol) and prop-2-enamide (444 mg, 6.24 mmol, CAS #9003-05-8) in THF (25 mL) was added t-BuOK (701 mg, 6.24 mmol) at 0° C. The mixture was then stirred at 20° C. for 2 hrs. On completion, the reaction mixture was quenched by adding it to sat. NH4C1 (80 mL) at 0° C., and then extracted with EA mL (80 mL×3). The combined organic layers were dried over Na₂SO₄, filtered and concentrated under reduced pressure to give a residue. The residue was purified by column chromatography (SiO₂, Petroleum ether/Ethyl acetate=2/1 to 1/1) to give the title compound (1.8 g, 72% yield) as a yellow solid. LC-MS (ESI⁺) m/z 391.9 (M+H)⁺; ¹H NMR (400 MHz, DMSO-d₆) 5=10.81 (s, 1H), 7.08-6.95 (m, 3H), 3.81 (dd, J=4.8, 12.0 Hz, 1H), 3.47 (s, 4H), 2.98-2.88 (m, 4H), 2.71-2.60 (m, 1H), 2.47-2.45 (m, 1H), 2.26-2.13 (m, 1H), 2.05-1.95 (m, 1H), 1.42 (s, 9H).

### Step 3—3-(3-Fluoro-4-(piperazin-1-yl)phenyl)piperidine-2,6-dione

To a solution of tert-butyl 4-[4-(2,6-dioxo-3-piperidyl)-2-fluoro-phenyl]piperazine-1-carboxylate (1.5 g, 3.8 mmol) in DCM (20 mL) was added HCl/dioxane (4 M, 5 mL). The mixture was then stirred at 20° C. for 1 hr. On completion, the reaction mixture was concentrated under reduced pressure to give a residue. Then sat. NaHCO₃ (15 mL) was added and the mixture was extracted with EA (30 mL×3). The combined organic layers were dried over Na₂SO₄, filtered and concentrated under reduced pressure to the title compound (1 g) as a yellow solid. LC-MS (ESI⁺) m/z 291.8 (M+H); ¹H NMR (400 MHz, DMSO-d₆) δ=10.80 (s, 1H), 7.03-6.99 (m, 1H), 6.98-6.93 (m, 2H), 3.79 (dd, J=4.8, 12.0 Hz, 1H), 2.93-2.87 (m, 4H), 2.85-2.80 (m, 4H), 2.70-2.58 (m, 1H), 2.46 (s, 1H), 2.19 (dq, J=4.4, 12.4 Hz, 1H), 2.02-1.97 (m, 1H).

### 3-(3-Fluoro-4-(4-oxopiperidin-1-yl)phenyl)piperidine-2,6-dione (Intermediate GA)

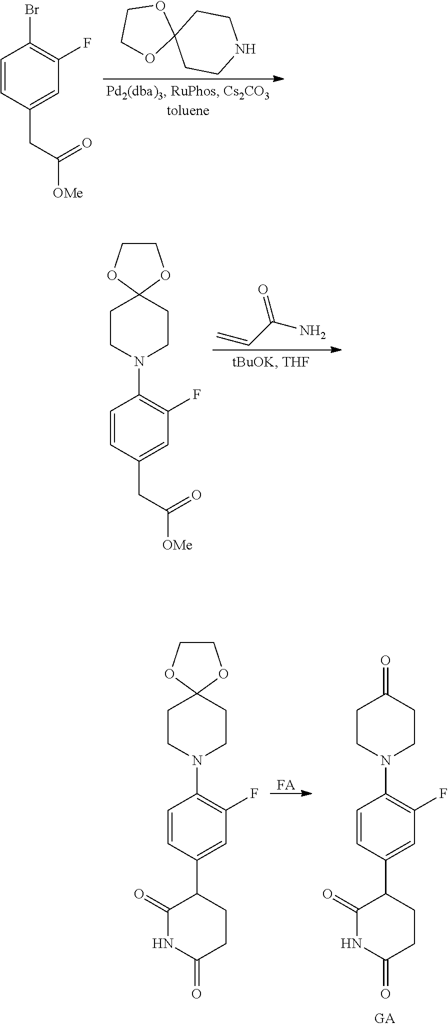

### Step 1—Methyl 2-(3-fluoro-4-(1,4-dioxa-8-azaspiro[4.5]decan-8-yl)phenyl)acetate

A mixture of methyl 2-(4-bromo-3-fluoro-phenyl)acetate (5 g, 20.2 mmol, CAS #942282-41-9), 1,4-dioxa-8-azaspiro[4.5]decane (2.90 g, 20.2 mmol, 2.59 mL, CAS #177-11-7), Cs₂CO₃ (19.8 g, 60.7 mmol), XPhos (1.16 g, 2.43 mmol) and Pd₂(dba)₃ (927 mg, 1.01 mmol) in toluene (15 mL) was degassed and purged with N₂ three times. Then the mixture was stirred at 100° C. for 2 hrs under N₂ atmosphere. On completion, the reaction mixture was quenched by addition of H₂O (10 mL), and then diluted with EA (10 mL) and extracted with EA (20 mL×2). The combined organic layers were washed with brine (20 mL×2), dried over Na₂SO₄, filtered and concentrated under reduced pressure to give a residue. The crude product was purified by reverse phase HPLC (0.1% FA condition) to give the title compound (650 mg, 8% yield. FA) as a white solid. LC-MS (ESI⁺) m/z 310.0 (M+H)⁺.

### Step 2—3-(3-Fluoro-4-(1,4-dioxa-8-azaspiro[4.5]decan-8-yl)phenyl)piperidine-2,6-dione

To a solution of methyl 2-[4-(1,4-dioxa-8-azaspiro[4.5]decan-8-yl)-3-fluoro-phenyl]acetate (600 mg, 1.94 mmol), prop-2-enamide (152 mg, 2.13 mmol, 147 μL) in THF (8 mL) was added t-BuOK (239 mg, 2.13 mmol). Then the mixture was stirred at 25° C. for 12 hrs. On completion, the reaction mixture was quenched by addition of H₂O (10 mL), and then diluted with EA (20 mL) and extracted with EA (20 mL×2). The combined organic layers were washed with brine (10 mL×2), dried over Na₂SO₄, filtered and concentrated under reduced pressure to give the title compound (550 mg) as a white solid. LC-MS (ESI⁺) m/z 349.1 (M+H)⁺.

### Step 3—3-(3-Fluoro-4-(4-oxopiperidin-1-yl)phenyl)piperidine-2,6-dione

To a solution of 3-[4-(1,4-dioxa-8-azaspiro[4.5]decan-8-yl)-3-fluoro-phenyl]piperidine-2,6-dione (550 mg, 1.58 mmol) in H₂O (2.5 mL) and formic acid (2.5 mL). The mixture was then stirred at 80° C. for 2 hrs. On completion, the reaction mixture was quenched by addition of H₂O (10 mL), and then diluted with EA (10 mL) and extracted with EA (15 mL×2). The combined organic layers were washed with brine (10 mL×2), dried over Na₂SO₄, filtered and concentrated under reduced pressure to give a residue. The crude product was purified by reverse phase HPLC (0.1% FA condition) to give the title compound (260 mg, 47% yield, FA) as a yellow solid. LC-MS (ESI⁺) m/z 305.2 (M+H)⁺.

### 3-(4-(4-(4-(4-Bromophenyl)piperazin-1-yl)piperidin-1-yl)-3-fluorophenyl)piperidine-2,6-dione (Intermediate GB)

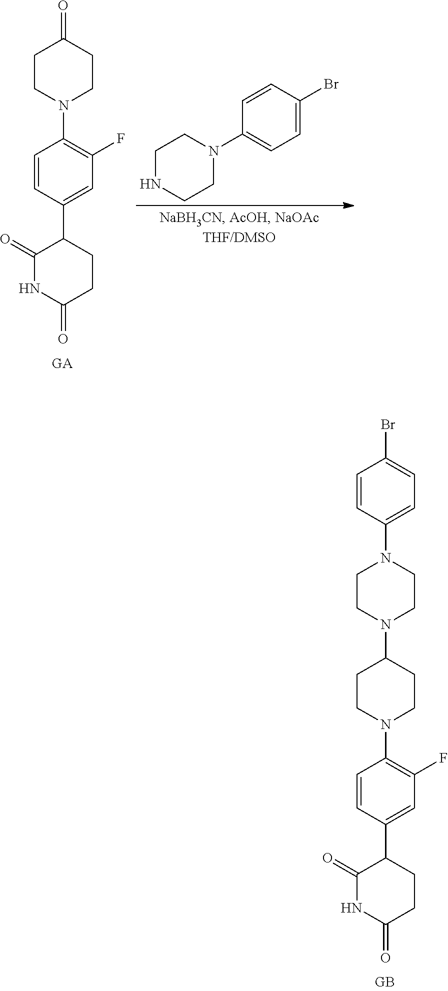

To a solution of 1-(4-bromophenyl)piperazine (274 mg, 986 μmol, CAS #66698-28-0) in DMSO (2.5 mL) and THF (2.5 mL) was added KOAc (290 mg. 2.96 mmol), HOAc (178 mg, 2.96 mmol, 169 μL) and 3-[3-fluoro-4-(4-oxo-1-piperidyl)phenyl]piperidine-2,6-dione (300 mg, 986 μmol, Intermediate GA). Then the mixture was stirred at 80° C. for 12 hrs. Next, NaBH₃CN (155 mg, 2.46 mmol) was added at 0° C., then the mixture was stirred at 25° C. for 12 hrs. On completion, the mixture was filtered and the mixture was concentrated under reduced pressure to give a residue. The mixture was purified by reverse phased HPLC (0.1% FA) to give the title compound (120 mg, 22% yield) as a white solid. LC-MS (ESI⁺) m/z 529.1 (M+H)⁺.

### 4-(2-Chloro-4-(piperazin-1-yl)phenyl)-7-fluoro-6-(1-isobutyryl-1,2,5,6-tetrahydropyridin-3-yl)-N,N-dimethyl-1H-indole-2-carboxamide (Intermediate GC)

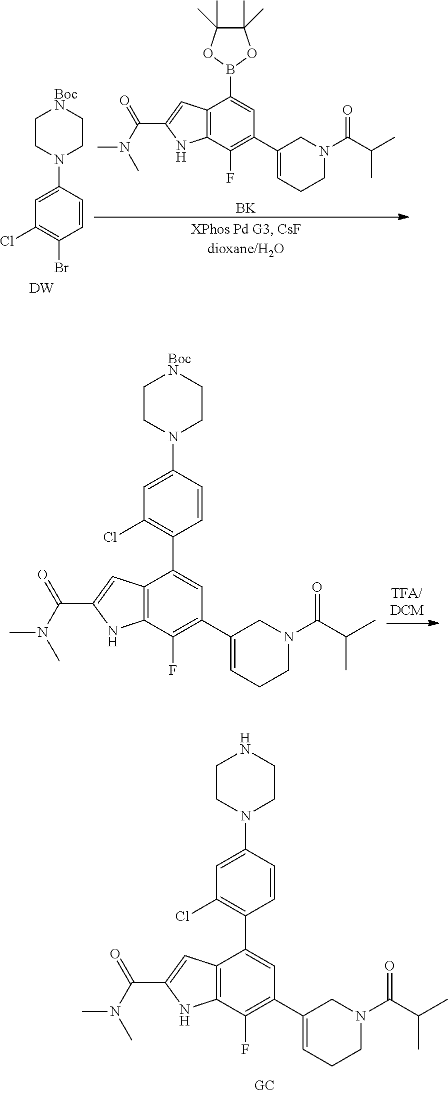

### Step 1—Tert-butyl 4-(3-chloro-4-(2-(dimethylcarbamoyl)-7-fluoro-6-(1-isobutyryl-1,2,5,6-tetrahydropyridin-3-yl)-1H-indol-4-yl)phenyl)piperazine-1-carboxylate

A mixture of tert-butyl 4-(4-bromo-3-chloro-phenyl)piperazine-1-carboxylate (300 mg, 799 μmol, Intermediate DW), 7-fluoro-N,N-dimethyl-6-[1-(2-methylpropanoyl)-3,6-dihydro-2H-pyridin-5-yl]-4-(4,4,5,5-tetramethyl-1,3,2-dioxaborolan-2-yl)-1H-indole-2-carboxamide (386 mg, 799 μmol, Intermediate BK), CsF (364 mg, 2.40 mmol, 88.4 μL), and XPhosPd G3 (67.6 mg, 79.9 μmol) in dioxane (4 mL) and H₂O (0.8 mL) was degassed and purged with N₂ three times. Then the mixture was stirred at 80° C. for 1 hr under N₂ atmosphere. On completion, the reaction mixture was filtered and concentrated under reduced pressure to give the title compound (450 mg) as a white solid. LC-MS (ESI⁺) m/z 652.0 (M+H)⁺.

### Step 2—4-(2-Chloro-4-(piperazin-1-yl)phenyl)-7-fluoro-6-(1-isobutyryl-1,2,5,6-tetrahydropyridin-3-yl)-N,N-dimethyl-1H-indole-2-carboxamide

To a solution of tert-butyl 4-[3-chloro-4-[2-(dimethylcarbamoyl)-7-fluoro-6-[1-(2-methylpropanoyl)-3,6-dihydro-2Hpyridin-5-yl]-1H-indol-4-yl]phenyl]piperazine-1-carboxylate (400 mg, 613 μmol) in DCM (4 mL) was added TFA (2 mL). The mixture was then stirred at 25° C. for 2 hrs. On completion, the reaction mixture was concentrated under reduced pressure to give a residue. The crude product was purified by reverse phase HPLC (neutral condition) to give the title compound (170 mg, 47% yield) as a white solid. LC-MS (ESI⁺) m/z 552.1 (M+H)⁺.

### 7-Bromo-5-chloro-N,N-dimethylbenzofuran-2-carboxamide (Intermediate GD)

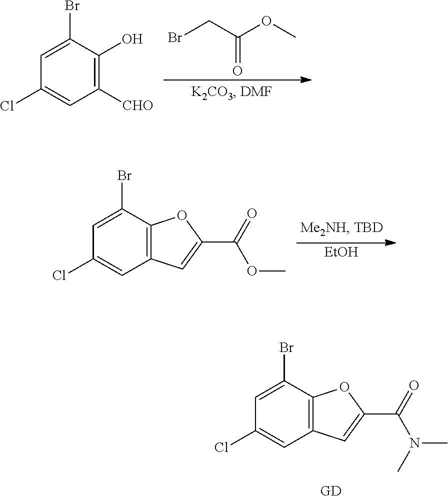

### Step 1—Methyl 7-bromo-5-chlorobenzofuran-2-carboxylate

To a solution of 3-bromo-5-chloro-2-hydroxybenzaldehyde (50.0 g, 212 mmol, CAS #19652-32-5) in DMF (500 mL) was added K₂CO₃ (117 g, 849 mmol) and methyl 2-bromoacetate (35.7 g, 233 mmol, CAS #96-32-2). Then the mixture was stirred at 80° C. for 15 hr under N₂ atmosphere. After completion, the reaction mixture was diluted with water (1.00 L) and extracted with EtOAc (1.00 L×3). The combined organic layers were washed with brine (1.00 L), dried over anhydrous Na₂SO₄, filtered and concentrated under reduced pressure to give a crude product. The crude product was triturated with Ethyl acetate/Petroleum ether=10:1 (1.00 L) to afford the title compound (38.0 g, 61% yield) as a yellow solid. LC-MS (ESI⁺) m/z 288.8 (M+H)⁺; ¹H NMR (400 MHz, DMSO-d₆) δ ppm 7.94-7.91 (m, 2H), 7.84 (s, 1H), 3.93 (s, 3H).

### Step 2—7-Bromo-5-chloro-N,N-dimethylbenzofuran-2-carboxamide

To a solution of methyl 7-bromo-5-chlorobenzofuran-2-carboxylate (20.0 g, 69.1 mmol) in EtOH (200 mL) was added TBD (2.88 g, 20.7 mmol) and N-methylmethanamine (2 M, 52.0 mL), then the mixture was stirred at 25° C. for 12 hr. After completion, the crude reaction mixture concentrated under reduced pressure to remove EtOH, and then diluted with EtOAc/water=1:1 (200 mL), filter to remove insoluble matters, and the filtrate was extracted with EtOAc (50.0 mL×3). The combined organic layers were concentrated under reduced pressure to give a crude product. The crude product was triturated with Ethyl acetate/Petroleum ether=5:1 (100 mL) and filtered to afford the title compound (16.0 g, 75% yield) as a yellow solid. LC-MS (ESI⁺) m/z 303.8 (M+H)⁺; ¹H NMR (400 MHz, DMSO-d₆) δ ppm 7.86 (d, J=2.0 Hz, 1H), 7.83-7.79 (m, 1H), 7.49 (s, 1H), 3.25 (s, 3H), 3.04 (s, 3H).

### 3-((3-Fluoro-4-(4-(4-(4,4,5,5-tetramethyl-1,3,2-dioxaborolan-2-yl)phenyl)piperidin-1-yl)phenyl)amino)piperidine-2,6-dione (Intermediate GE)

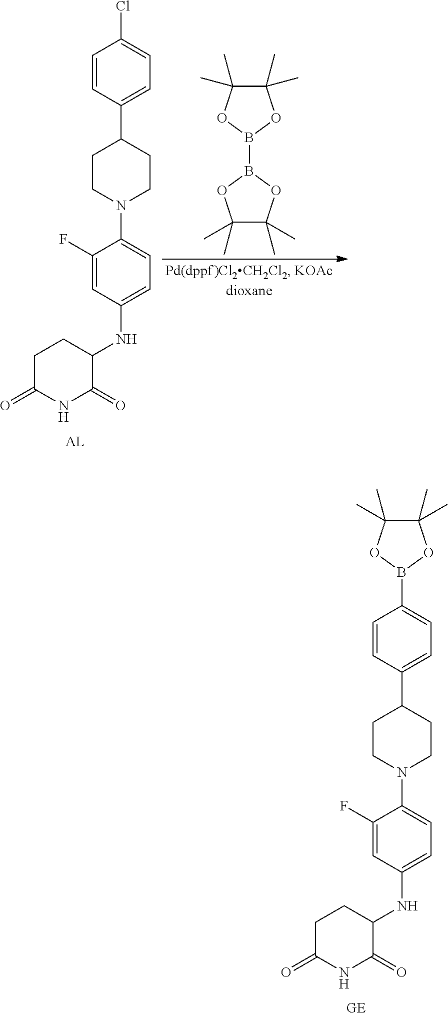

To a mixture of 3-[4-[4-(4-bromophenyl)-1-piperidyl]-3-fluoro-anilino]piperidine-2,6-dione (0.50 g. 1.1 mmol, Intermediate AL) and 4,4,5,5-tetramethyl-2-(4,4,5,5-tetramethyl-1,3,2-dioxaborolan-2-yl)-1,3,2-dioxaborolane (0.55 g, 2.2 mmol) in dioxane (2.0 mL) was added Pd(dppf)Cl₂·CH₂Cl₂ (89 mg, 0.11 mmol) and KOAc (0.21 g, 2.17 mmol). Then the mixture was degassed and purged with N₂ three times, and the mixture was stirred at 90° C. for 2 hrs under N₂ atmosphere. On completion, the reaction mixture was concentrated under reduced pressure to give a residue. The crude product was purified by MPLC (SiO₂, PE:EA=10:1 to 3:1) to give title compound (0.30 g, 51% yield) as a brown solid. LC-MS (ESI⁺) m/z 508.2 (M+H)⁺; ¹H NMR (400 MHz, CHLOROFORM-d) δ=7.79 (d, J=8.0 Hz, 2H), 7.30 (d, J=8.0 Hz, 2H), 6.97-6.90 (m, 1H), 6.48-6.42 (m, 2H), 4.61 (s, 1H), 4.02 (d, J=12.0 Hz, 1H), 3.42 (d, J=10.0 Hz, 2H), 2.94-2.85 (m, 1H), 2.81-2.72 (m, 3H), 2.68-2.60 (m, 1H), 2.59-2.52 (m, 1H), 2.10-1.82 (m, 6H), 1.35 (s, 12H).

### 2-Methyl-1-(5-(4,4,5,5-tetramethyl-1,3,2-dioxaborolan-2-yl)-3,6-dihydropyridin-1 (2H)-yl)-3-(1H-1,2,3-triazol-1-yl)propan-1-one (Intermediate GF)

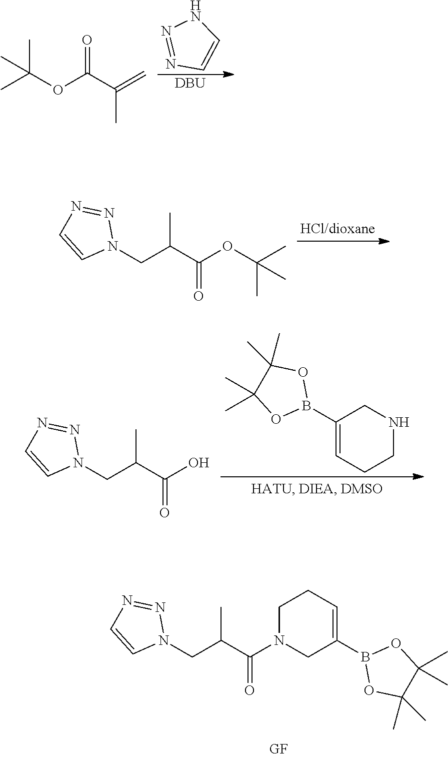

### Step 1—Tert-butyl 2-methyl-3-(1H-1,2,3-triazol-1-yl)propanoate

To a solution of tert-butyl methacrylate (10 g, 70.3 mmol, CAS #585-07-9) and 1H-1,2,3-triazole (4.86 g, 70.3 mmol, CAS #288-35-7) was added DBU (5.35 g, 35.2 mmol). The mixture was then stirred at 90° C. for 4 hrs. On completion, the reaction mixture was filtered and the filtrate was concentrated in vacuo to give the crude residue. The crude residue was purified by reverse phase HPLC (0.1% FA condition) to give the title compound (3.5 g, 22% yield) as white solid. LC-MS (ESI+) m/z=212.0 (M+H)⁺.

### Step 2—2-Methyl-3-(1H-1,2,3-triazol-1-yl)propanoic acid

To a solution of tert-butyl 2-methyl-3-(1H-1,2,3-triazol-1-yl)propanoate (3.3 g, 16 mmol) in HCl/dioxane (45 mL). The mixture was then stirred at 25° C. for 1 hr. On completion, the reaction mixture was concentrated in vacuo to give the title compound (3.5 g, HCl) as a white solid. LC-MS (ESI⁺) m/z=156.0 (M+H)⁺.

### Step 3—2-Methyl-1-(5-(4,4,5,5-tetramethyl-1,3,2-dioxaborolan-2-yl)-3,6-dihydropyridin-1 (2H)-yl)-3-(1H-1,2,3-triazol-1-yl)propan-1-one

A mixture of 2-methyl-3-(1H-1,2,3-triazol-1-yl)propanoic acid (3 g. 19.3 mmol) in DMSO (40 mL) was added HATU (11.0 g, 29.0 mmol) and DIEA (12.5 g, 96.7 mmol). Then 5-(4,4,5,5-tetramethyl-1,3,2-dioxaborolan-2-yl)-1,2,3,6-tetrahydropyridine (4.75 g, 19.3 mmol, synthesized via Step 1 of Intermediate E) was added and the mixture was stirred at 25° C. for 1 hr. On completion, the reaction mixture was poured into water (200 mL), filtered and the filter cake was dried under reduced pressure to give the title compound (4 g) as a brown solid. LC-MS (ESI⁺) m/z=346.9 (M+H)⁺.

### (R)-4-chloro-7-fluoro-N,N-dimethyl-6-(1-(2-methyl-3-(1H-1,2,3-triazol-1-yl)propanoyl)-1,2,5,6-tetrahydropyridin-3-yl)-1H-indole-2-carboxamide (Intermediate GG) & (S)-4-chloro-7-fluoro-N,N-dimethyl-6-(1-(2-methyl-3-(1H-1,2,3-triazol-1-yl)propanoyl)-1,2,5,6-tetrahydropyridin-3-yl)-1H-indole-2-carboxamide (Intermediate GH)

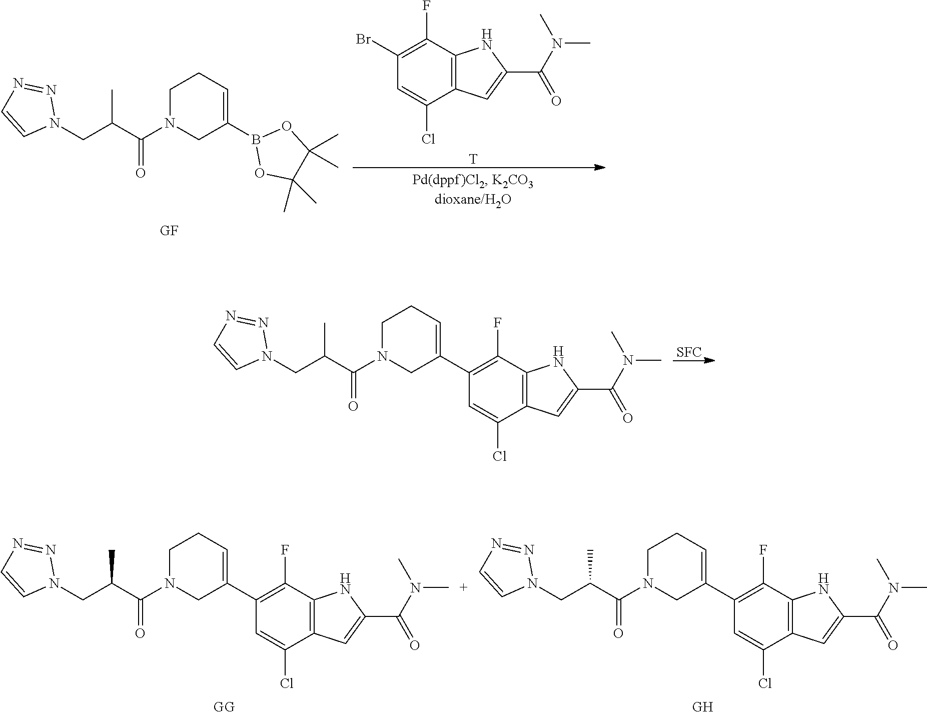

### Step 1—4-Chloro-7-fluoro-N,N-dimethyl-6-(1-(2-methyl-3-(1H-1,2,3-triazol-1-yl)propanoyl)-1,2,5,6-tetrahydropyridin-3-yl)-1H-indole-2-carboxamide

A mixture of 2-methyl-1-(5-(4,4,5,5-tetramethyl-1,3,2-dioxaborolan-2-yl)-3,6-dihydropyridin-1 (2H)-yl)-3-(1H-1,2,3-triazol-1-yl)propan-1-one (1.50 g, 4.33 mmol, Intermediate GF) and 6-bromo-4-chloro-7-fluoro-N,N-dimethyl-TH-indole-2-carboxamide (1.38 g, 4.33 mmol, Intermediate T) in dioxane (15 mL) and H₂O (0.3 mL) was added Pd(dppf)Cl₂·CH₂Cl₂ (354 mg, 433 μmol) and K₂CO₃ (1.20 g, 8.66 mmol). The mixture was stirred at 80° C. for 2 hrs. On completion, the reaction mixture was diluted with water (200 mL) and extracted with ethyl acetate (3×70 mL). The combined organic layers were washed with brine (200 mL) and dried over anhydrous sodium sulfate, filtered and the filtrate was concentrated in vacuo to give the crude residue. The crude residue was purified by column chromatography (SiO₂, Petroleum ether/Ethyl acetate=100/1 to 5/1) to give the title compound (1.1 g, 54% yield) as a yellow solid. LC-MS (ESI⁺) m/z 459.0 (M+H)⁺.

### Step 2—(R)-4-chloro-7-fluoro-N,N-dimethyl-6-(1-(2-methyl-3-(1H-1,2,3-triazol-1-yl)propanoyl)-1,2,5,6-tetrahydropyridin-3-yl)-1H-indole-2-carboxamide) & (S)-4-chloro-7-fluoro-N,N-dimethyl-6-(1-(2-methyl-3-(1H-1,2,3-triazol-1-yl)propanoyl)-1,2,5,6-tetrahydropyridin-3-yl)-1H-indole-2-carboxamide

To a solution of 4-chloro-7-fluoro-N,N-dimethyl-6-(1-(2-methyl-3-(1H-1,2,3-triazol-1-yl)propanoyl)-1,2,5,6-tetrahydropyridin-3-yl)-1H-indole-2-carboxamide (1.1 g, 2.40 mmol) was isolated by column: DAICEL CHIRALPAK IA (250 mm×30 mm, 10 um); mobile phase: [HEXANE-IPA (0.1% IPAM)]: B %:40%, isocratic elution mode to give (R)-4-chloro-7-fluoro-N,N-dimethyl-6-(1-(2-methyl-3-(1H-1,2,3-triazol-1-yl)propanoyl)-1,2,5,6-tetrahydropyridin-3-yl)-1H-indole-2-carboxamide) (400 mg) as a brown solid and (S)-4-chloro-7-fluoro-N,N-dimethyl-6-(1-(2-methyl-3-(1H-1,2,3-triazol-1-yl)propanoyl)-1,2,5,6-tetrahydropyridin-3-yl)-1H-indole-2-carboxamide (460 mg) as a brown solid. LC-MS (ESI⁺) m/z 459.0 (M+H)⁺ for both isomers. The absolute stereochemistry of the enantiomers was assigned arbitrarily.

### 4-(4-(4-(4-Bromophenyl)piperazin-1-yl)piperidin-1-yl)-5-fluoro-2-methoxyaniline (Intermediate GI)

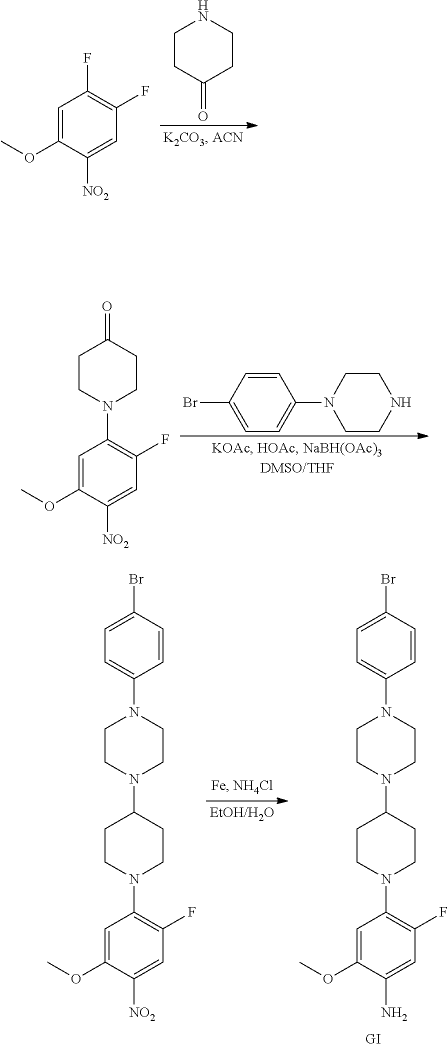

### Step 1—1-(2-fluoro-5-methoxy-4-nitrophenyl)piperidin-4-one

To a solution of 1,2-difluoro-4-methoxy-5-nitrobenzene (5 g. 26.4 mmol, synthesized via Step 1 of Intermediate AQ), piperidin-4-one (2.62 g, 26.4 mmol, CAS #41661-47-6) in ACN (30 mL) was added K₂CO₃ (18.2 g, 132 mmol). The mixture was then stirred at 80° C. for 2 hrs. On completion, the mixture was quenched with water (30 mL) and extracted with EA (3×30 mL). The combined organic layers were washed with aqueous NaCl (30 mL) and dried over anhydrous sodium sulfate, filtered and the filtrate was concentrated in vacuo to give the title compound (5 g) as yellow solid. LC-MS (ESI⁺) n/z 269.1 (M+H)⁺.

### Step 2—1-(4-Bromophenyl)-4-(1-(2-fluoro-5-methoxy-4-nitrophenyl)piperidin-4-yl)piperazine

To a solution of 1-(2-fluoro-5-methoxy-4-nitrophenyl)piperidin-4-one (500 mg, 1.86 mmol), 1-(4-bromophenyl)piperazine (449 mg, 1.86 mmol, CAS #66698-28-0) in DMSO (5 mL) and THF (5 mL) was added KOAc (548 mg, 5.59 mmol), AcOH (335 mg, 5.59 mmol, 320 L) and 4 Å molecular sieves (500 mg, 1.00 mol). Then the mixture was stirred at 25° C. for 12 hrs. Next, to the mixture was added NaBH(OAc)₃ (1.19 g, 5.59 mmol) at 0° C., and the mixture was stirred at 25° C. for 1 hr. On completion, the mixture was quenched with water (20 mL) and filtered and the filtrate cake give the crude residue. The residue was purified by column chromatography (SiO₂, Petroleum ether:Ethyl acetate=3/1 to 1/1) to give the title compound (600 mg, 65% yield) as yellow solid. LC-MS (ESI⁺) m/z 495.2 (M+H)⁺.

### Step 3—4-(4-(4-(4-Bromophenyl)piperazin-1-yl)piperidin-1-yl)-5-fluoro-2-methoxyaniline

To a solution of 1-(4-bromophenyl)-4-(1-(2-fluoro-5-methoxy-4-nitrophenyl)piperidin-4-yl)piperazine (600 mg, 1.22 mmol) in EtOH (10 mL) and H₂O (5 mL) was added Fe (679 mg, 12.16 mmol) and NH4Cl (650 mg, 12.1 mmol). Then the mixture was stirred at 80° C. for 1 hr. On completion, the mixture was filtered and the filtrate was concentrated in vacuo to give the crude residue. The residue was extracted by ethyl acetate (2×20 mL). The combined organic layers were washed by brine (40 mL) and dried over anhydrous sodium sulfate, filtered and the filtrate was concentrated in vacuo to give the title compound (500 mg, 89% yield) as gray solid. LC-MS (ESI⁺) m/z 463.2 (M+H)⁺.

### 3-((4-(4-(4-(4-Bromophenyl)piperazin-1-yl)piperidin-1-yl)-5-fluoro-2-methoxyphenyl)amino)piperidine-2,6-dione (Intermediate GJ)

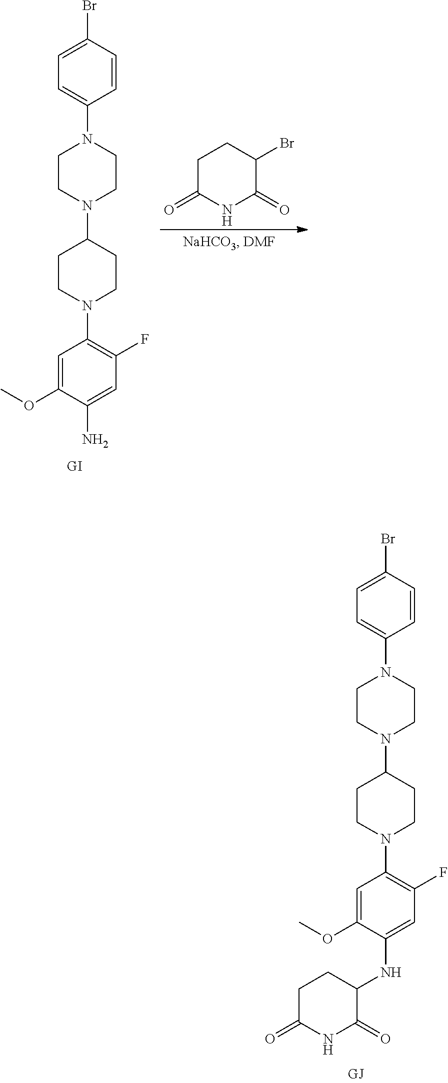 [SMILES: COc1cc(N2CCC(N3CCN(c4ccc(Br)cc4)CC3)CC2)c(F)cc1N ; O=C1CCC(Br)C(=O)N1 ; COc1cc(N2CCC(N3CCN(c4ccc(Br)cc4)CC3)CC2)c(F)cc1NC1CCC(=O)NC1=O]

To a solution of 4-(4-(4-(4-bromophenyl)piperazin-1-yl)piperidin-1-yl)-5-fluoro-2-methoxyaniline (500 mg, 1.08 mmol, Intermediate GI) and 3-bromopiperidine-2,6-dione (621 mg, 3.24 mmol, CAS #62595-74-8) in DMF (10 mL) was added NaHCO₃ (453 mg, 5.40 mmol, 209 μL). Then the mixture was stirred at 80° C. for 12 hrs. On completion, the mixture was concentrated under reduced pressure to remove solvent. The residue was purified by reverse phase HPLC (0.1% FA condition) to give the title compound (80 mg, 13% yield) as a white solid. LC-MS (ESI⁺) m/z 576.2 (M+H)⁺.

### 1-(4-Bromo-3-chloro-2-fluorophenyl)piperazine (Intermediate GK)

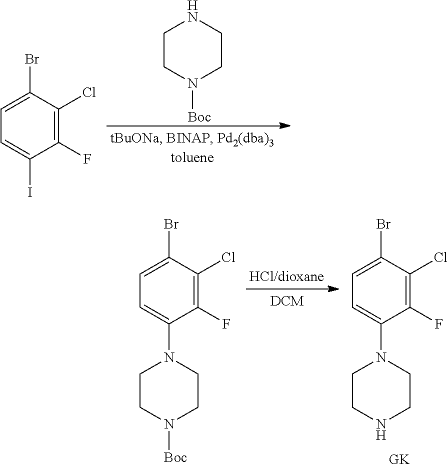 [SMILES: Fc1c(I)ccc(Br)c1Cl ; CC(C)(C)OC(=O)N1CCNCC1 ; CC(C)(C)OC(=O)N1CCN(c2ccc(Br)c(Cl)c2F)CC1 ; Fc1c(N2CCNCC2)ccc(Br)c1Cl]

### Step 1—Tert-butyl 4-(4-bromo-3-chloro-2-fluorophenyl)piperazine-1-carboxylate

To a solution of 1-bromo-2-chloro-3-fluoro-4-iodobenzene (4.8 g, 14.3 nmol CAS #883499-24-9) and tert-butyl piperazine-1-carboxylate (2.67 g, 14.3 mmol, CAS #57260-71-2) in toluene (50 mL) was added BINAP (891 mg, 1.43 mmol), tBuONa (2.75 g, 28.6 mmol), 4 Å molecular sieves (1 g), and Pd₂(dba)₃ (1.31 g, 1.43 mmol) at 25° C. Then the mixture was stirred at 90° C. for 2 hrs. On completion, the reaction mixture was filtered and concentrated in vacuo to give the crude residue. The residue was purified by column chromatography (SiO₂, Petroleum ether/Ethyl acetate=1/0 to 5/1) to give the title compound (2.8 g, 41% yield) as a yellow solid. LC-MS (ESI⁺) n/z 336.9 (M−55)⁺.

### Step 2—1-(4-Bromo-3-chloro-2-fluorophenyl)piperazine

To a solution of tert-butyl 4-(4-bromo-3-chloro-2-fluorophenyl)piperazine-1-carboxylate (2.8 g, 7.1 mmol) in DCM (20 mL) was added HCl/dioxane (6 M, 4.31 mL) at 25° C., then the mixture was stirred at 25° C. for 1 hr. On completion, the reaction mixture was concentrated in vacuo to give the title compound (2.5 g, HCl) as a yellow solid. LC-MS (ESI⁺) m/z 294.9 (M+H)⁺.

### 4-(4-(4-Bromo-3-chloro-2-fluorophenyl)piperazin-1-yl)-5-fluoro-2-methoxyaniline (Intermediate GL)

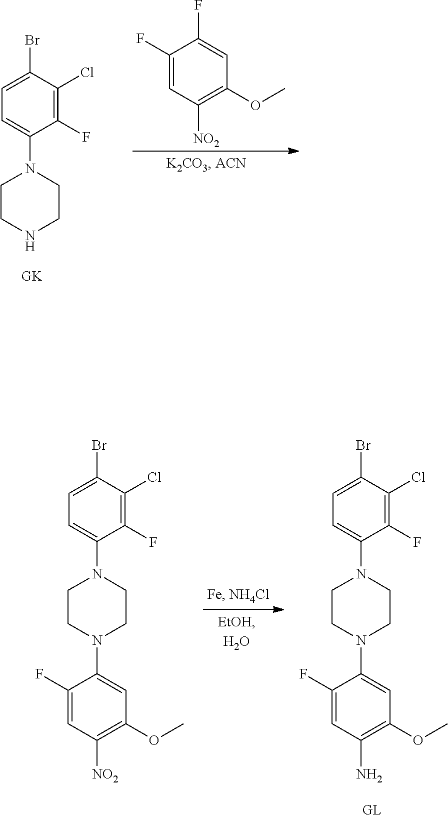

### Step 1—1-(4-Bromo-3-chloro-2-fluorophenyl)-4-(2-fluoro-5-methoxy-4-nitrophenyl)piperazine

To a solution of 1-(4-bromo-3-chloro-2-fluorophenyl)piperazine (1.5 g, 4.9 mmol, 19 L, HCl, Intermediate GK) and 1,2-difluoro-4-methoxy-5-nitrobenzene (860 mg, 4.55 mmol, synthesized via Step 1 of Intermediate AQ) in DMF (10 mL) was added K₂CO₃ (3.14 g, 22.7 mmol) at 25° C., then the mixture was stirred at 80° C. for 2 hrs. On completion, the reaction mixture was quenched with water (50 mL) and extracted with ethyl acetate (50×3 mL). The extracts were washed with brine (500 mL) and dried over anhydrous sodium sulfate, filtered and concentrated in vacuo to give the title compound (300 mg) as a yellow solid. LC-MS (ESI⁺) m/z 461.9 (M+H)⁺.

### Step 2—4-(4-(4-Bromo-3-chloro-2-fluorophenyl)piperazin-1-yl)-5-fluoro-2-methoxyaniline

To a solution of 1-(4-bromo-3-chloro-2-fluorophenyl)-4-(2-fluoro-5-methoxy-4-nitrophenyl)piperazine (230 mg, 497 μmol) in EtOH (2 mL) and H₂O (2 mL) was added Fe (167 mg, 2.98 mmol) and NH₄Cl (266 mg, 4.97 mmol) at 25° C. Then the mixture was stirred at 80° C. for 1 hr. On completion, the reaction mixture was filtered and the filter liquor was concentrated in vacuo to give the title compound (220 mg) as a green solid. LC-MS (ESI⁺) m/z 431.8 (M+H)⁺.

### 3-((4-(4-(4-Bromo-3-chloro-2-fluorophenyl)piperazin-1-yl)-5-fluoro-2-methoxyphenyl)amino)piperidine-2,6-dione (Intermediate GM)

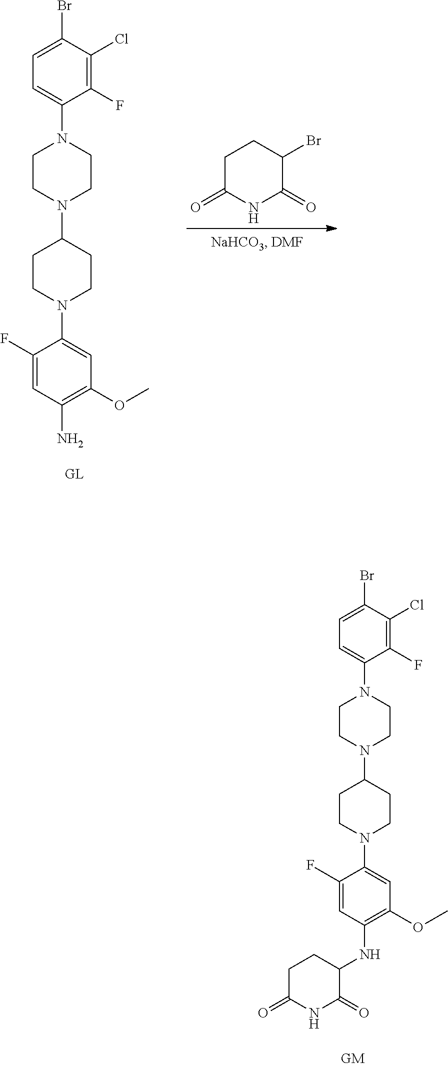 [SMILES: COc1cc(N2CCC(N3CCN(c4ccc(Br)c(Cl)c4F)CC3)CC2)c(F)cc1N ; O=C1CCC(Br)C(=O)N1 ; COc1cc(N2CCC(N3CCN(c4ccc(Br)c(Cl)c4F)CC3)CC2)c(F)cc1NC1CCC(=O)NC1=O]

To a solution of 4-(4-(4-bromo-3-chloro-2-fluorophenyl)piperazin-1-yl)-5-fluoro-2-methoxyaniline (200 mg, 462 μmol, Intermediate GL) and 3-bromopiperidine-2,6-dione (107 mg, 555 μmol, CAS #62595-74-8) in DMF (2 mL) was added NaHCO₃ (77.7 mg, 924 μmol, 36.0 μL) at 25° C., then the mixture was stirred at 80° C. for 5 hrs. On completion, the reaction mixture was quenched with water (15 mL) and extracted with ethyl acetate (30×3 mL). The extracts were washed with brine (500 mL) and dried over anhydrous sodium sulfate, filtered and concentrated in vacuo to give the crude residue. The residue was purified by column chromatography (SiO₂, Petroleum ether/Ethyl acetate=5/1 to 1/1) to give the title compound (180 mg, 69% yield) as a green solid. LC-MS (ESI⁺) m/z 545.0 (M+H)⁺.

### 4-(4-(4-Bromo-2-fluorophenyl)piperazin-1-yl)-3-fluoroaniline (Intermediate GN)

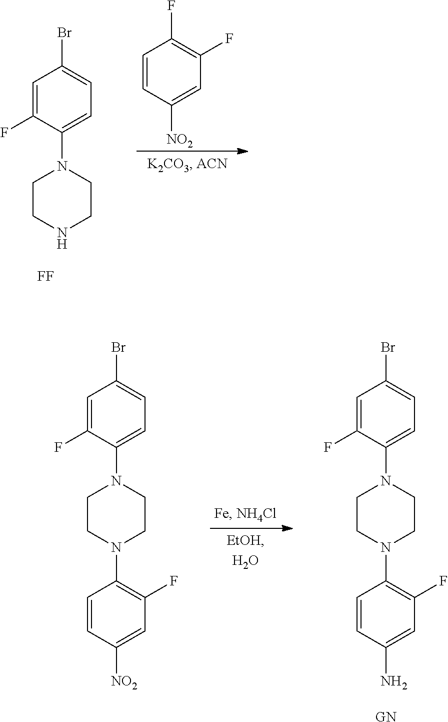

### Step 1—1-(4-Bromo-2-fluorophenyl)-4-(2-fluoro-4-nitrophenyl)piperazine

A solution of 1-(4-bromo-2-fluorophenyl)piperazine (2.00 g, 7.72 mmol, Intermediate FF), 1,2-difluoro-4-nitrobenzene (2.46 g, 15.4 mmol, 1.71 mL, CAS #369-34-6), and K₂CO₃ (3.20 g, 23.3 mmol) in ACN (20 mL) was stirred at 80° C. for 2 hrs. On completion, the reaction mixture was filtered and the filtrate was concentrated in vacuo to give the crude residue. Then the residue was purified by column chromatography (SiO₂, Petroleum ether:Ethyl acetate=10/1 to 3/1) to give the title compound (1.40 g, 45% yield) as a yellow solid. LC-MS (ESI⁺) m/z 397.7 (M+H)⁺.

### Step 2—4-(4-(4-Bromo-2-fluorophenyl)piperazin-1-yl)-3-fluoroaniline

A solution of 1-(4-bromo-2-fluorophenyl)-4-(2-fluoro-4-nitrophenyl)piperazine (1.40 g, 3.52 mmol). Fe (1.96 g, 35.2 mmol), and NH4Cl (1.88 g, 35.2 mmol) in EtOH (140 mL) and H₂O (40 mL) was stirred at 80° C. for 2 hrs. On completion, the residue was purified by column chromatography (SiO₂, Petroleum ether:Ethyl acetate=10/1 to 3/1) to give the title compound (800 mg, 62% yield) as a yellow solid. ¹H NMR (400 MHz, DMSO-d₆) δ=7.44 (dd, J=2.4, 12.4 Hz, 1H), 7.31 (td, J=1.2, 8.8 Hz, 1H), 7.03 (t, J=9.2 Hz, 1H), 6.82 (dd, J=8.4, 9.6 Hz, 1H), 6.39-6.27 (m, 2H), 5.06-4.92 (m, 2H), 3.15-3.07 (m, 4H), 3.01-2.93 (m, 4H).

### 3-((4-(4-(4-Bromo-2-fluorophenyl)piperazin-1-yl)-3-fluorophenyl)amino)piperidine-2,6-dione (Intermediate GO)

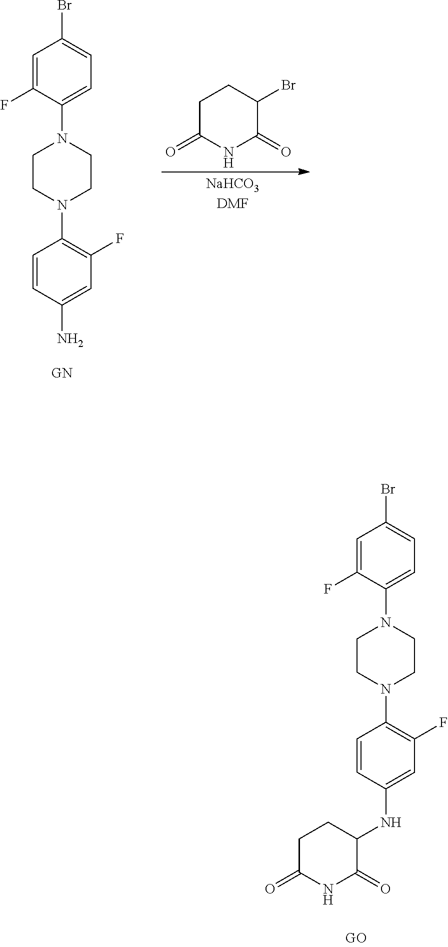

A solution of 4-(4-(4-bromo-2-fluorophenyl)piperazin-1-yl)-3-fluoroaniline (800 mg, 2.17 mmol, Intermediate GN), 3-bromopiperidine-2,6-dione (834 mg, 4.35 mmol, CAS #62595-74-8), and NaHCO₃ (365 mg, 4.35 mmol) in DMF (8 mL) was stirred at 80° C. for 2 hrs. On completion, the reaction mixture was purified by reverse phase HPLC (0.8 g/L ammonium bicarbonate). Then the crude product was triturated with DCM at 25° C. for 0.5 hours, then the mixture was filtered and the filter cake was dried in vacuo to give the title compound (640 mg, 60% yield) as a green solid. LC-MS (ESI⁺) m/z 479.1 (M+H)⁺.

### 4-(4-(4-Bromo-2,5-difluorophenyl)piperazin-1-yl)-3-fluoroaniline (Intermediate GP)

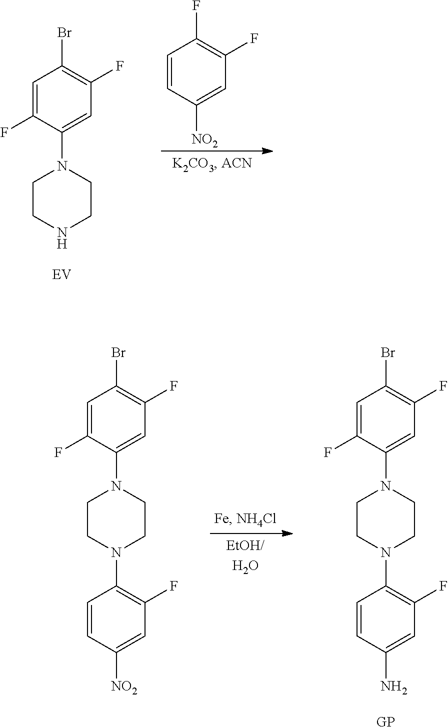

### Step 1—1-(4-Bromo-2,5-difluorophenyl)-4-(2-fluoro-4-nitrophenyl)piperazine

To a solution of 1-(4-bromo-2,5-difluorophenyl)piperazine (1.5 g, 4.78 mmol, HCl, Intermediate EV) in ACN (15 mL) was added K₂CO₃ (1.98 g, 14.3 mmol) and 1,2-difluoro-4-nitrobenzene (913 mg, 5.74 mmol, 635 mL, CAS #368-34-6). Then the mixture was stirred at 80° C. for 2 hrs. On completion, the reaction mixture was filtered and concentrated in vacuo to give the title compound (1.7 g) as a yellow solid. LC-MS (ESI⁺) m/z 416.0 (M+H)⁺.

### Step 2—4-(4-(4-Bromo-2,5-difluorophenyl)piperazin-1-yl)-3-fluoroaniline

To a solution of 1-(4-bromo-2,5-difluorophenyl)-4-(2-fluoro-4-nitrophenyl)piperazine (1.7 g, 4.08 mmol) in EtOH (36 mL) and H₂O (12 mL) was added Fe (1.14 g, 20.4 mmol) and NH₄Cl (1.09 g, 20.4 mmol) at 0° C. Then the mixture was stirred at 80° C. for 2 hrs. On completion, the reaction mixture was filtered and concentrated in vacuo. The residue was purified by prep-HPLC (0.8 g/L ammonium bicarbonate) to give the title compound (1.5 g, 91% yield) as a white solid. LC-MS (ESI⁺) m/z 386.9 (M+H)⁺.

### 3-((4-(4-(4-Bromo-2,5-difluorophenyl)piperazin-1-yl)-3-fluorophenyl)amino)piperidine-2,6-dione (Intermediate GQ)

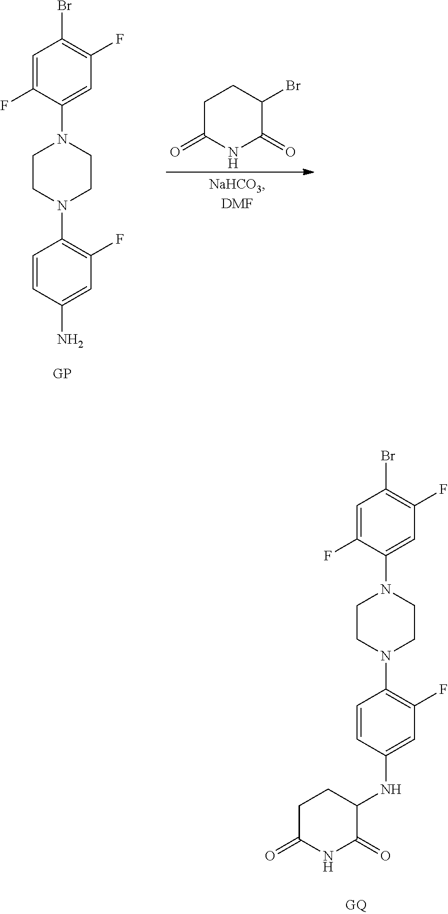

To a solution of 4-(4-(4-bromo-2,5-difluorophenyl)piperazin-1-yl)-3-fluoroaniline (1 g, 2.59 mmol, Intermediate GP) in DMF (10 mL) was added NaHCO₃ (1.09 g, 12.9 mmol, 503 μL) and 3-bromopiperidine-2,6-dione (596 mg, 3.11 mmol, CAS #62595-74-8). The mixture was then stirred at 80° C. for 48 hrs. On completion, the reaction mixture was concentrated in vacuo to give the crude residue. The crude residue was purified by reverse phase HPLC (0.8 g/L ammonium bicarbonate) to give the title compound (450 mg, 35% yield) as a white solid. LC-MS (ESI⁺) m/z 497.0 (M+H)⁺.

### 6-(1-(3-(1H-pyrazol-1-yl)propanoyl)-1,2,5,6-tetrahydropyridin-3-yl)-4-(2-chloro-5-fluoro-4-(piperazin-1-yl)phenyl)-7-fluoro-N,N-dimethyl-1H-indole-2-carboxamide (Intermediate GR)

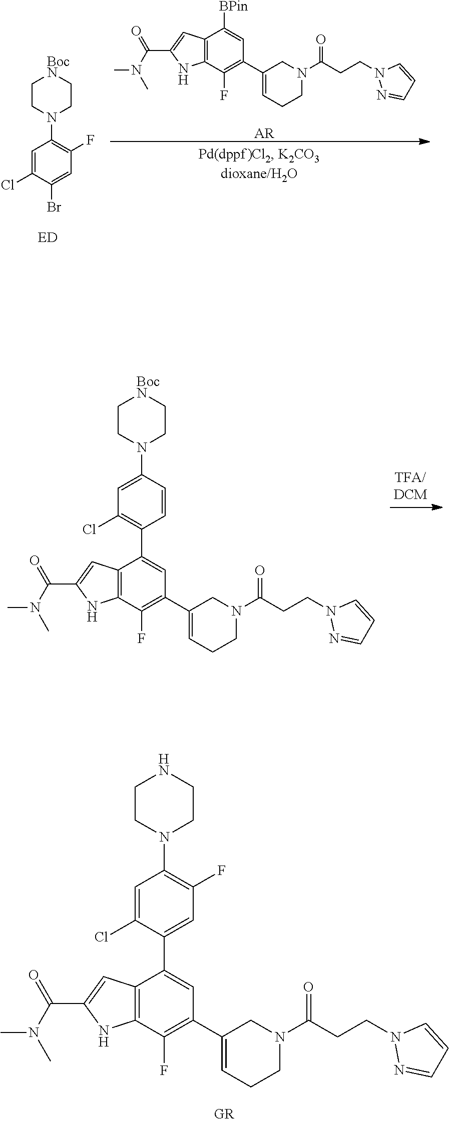

### Step 1—Tert-butyl 4-(4-(6-(1-(3-(1H-pyrazol-1-yl)propanoyl)-1,2,5,6-tetrahydropyridin-3-yl)-2-(dimethylcarbamoyl)-7-fluoro-1H-indol-4-yl)-5-chloro-2-fluorophenyl)piperazine-1-carboxylate

A mixture of tert-butyl 4-(4-bromo-5-chloro-2-fluoro-phenyl)piperazine-1-carboxylate (700 mg, 1.78 mmol, Intermediate ED), 7-fluoro-N,N-dimethyl-6-[1-(3-pyrazol-1-ylpropanoyl)-3,6-dihydro-2H-pyridin-5-yl]-4-(4,4,5,5-tetramethyl-1,3,2-dioxaborolan-2-yl)-1H-indole-2-carboxamide (1.05 g, 1.96 mmol, Intermediate AR), Pd(dppf)Cl₂·CH₂Cl₂ (145 mg, 178 μmol), and K₂CO₃ (737 mg, 5.33 mmol) in dioxane (4 mL) and H₂O (1 mL) was degassed and purged with N₂ three times. Then the mixture was stirred at 80° C. for 1 hr under N₂ atmosphere. On completion, the reaction mixture was concentrated under reduced pressure to remove solvent. The residue was purified by column chromatography (SiO₂, Petroleum ether:Ethyl acetate=3/1 to Ethyl acetate:THF-1/1) to give the title compound (1.0 g, 66% yield) as a yellow solid. LC-MS (ESI⁺) m/z 722.2 (M+H)⁺.

### Step 2—6-(1-(3-(1H-pyrazol-1-yl)propanoyl)-1,2,5,6-tetrahydropyridin-3-yl)-4-(2-chloro-5-fluoro-4-(piperazin-1-yl)phenyl)-7-fluoro-N,N-dimethyl-1H-indole-2-carboxamide

To a solution of tert-butyl 4-[5-chloro-4-[2-(dimethylcarbamoyl)-7-fluoro-6-[1-(3-pyrazol-1-ylpropanoyl)-3,6-dihydro-2H-pyridin-5-yl]-1H-indol-4-yl]-2-fluoro-phenyl]piperazine-1-carboxylate (1.00 g, 1.38 mmol) in DCM (8 mL) was added TFA (3.07 g, 26.9 mmol). The mixture was then stirred at 25° C. for 1 hr. On completion, the reaction mixture was concentrated under N₂ to remove solvent. The crude residue was purified by reverse phase HPLC (0.1% FA condition) to give the title compound (800 mg, 86% yield, FA) as a yellow solid. LC-MS (ESI⁺) m/z 622.2 (M+H)⁺.

### 1-(4-Chloro-3-cyclopropylphenyl)piperazine (Intermediate GS)

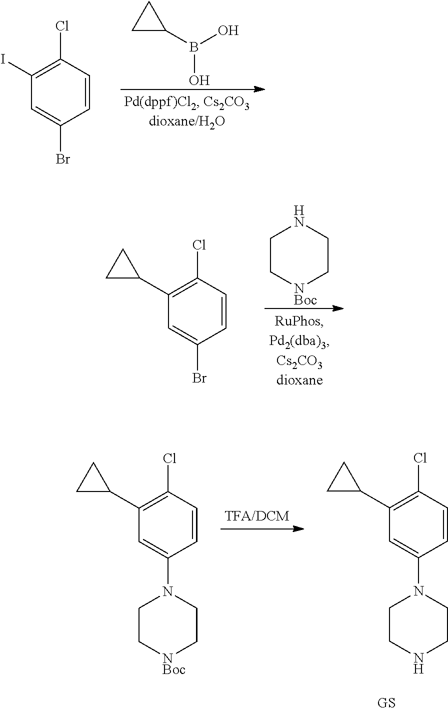 [SMILES: Clc1ccc(Br)cc1I ; OB(O)C1CC1 ; Clc1ccc(Br)cc1C1CC1 ; CC(C)(C)OC(=O)N1CCNCC1 ; CC(C)(C)OC(=O)N1CCN(c2ccc(Cl)c(C3CC3)c2)CC1 ; Clc1ccc(N2CCNCC2)cc1C1CC1]

### Step 1—4-Bromo-1-chloro-2-cyclopropylbenzene

A mixture of 4-bromo-1-chloro-2-iodobenzene (5 g, 15.7 mmol, CAS #774608-49-0), cyclopropylboronic acid (1.35 g, 15.7 mmol, CAS #411235-57-9), Pd(dppf)Cl₂·CH₂Cl₂ (1.29 g, 1.58 mmol), and Cs₂CO₃ (15.4 g, 47.2 mmol) in dioxane (40 mL) and H₂O (8 mL) was degassed and purged with N₂ three times. Then the mixture was stirred at 100° C. for 12 hr under N₂ atmosphere. On completion, the reaction mixture was filtered and concentrated under reduced pressure to give a residue. The residue was purified by column chromatography (SiO₂, Petroleum ether/Ethyl acetate=1:0) to give the title compound (1.4 g, 83% yield) as a colorless oil. ¹H NMR (400 MHz, DMSO-d₆) δ=7.37 (d, J=1.2 Hz, 2H), 7.18 (s, 1H), 2.17-2.06 (m, 1H), 1.04-0.98 (m, 2H), 0.78-0.73 (m, 2H).

### Step 2—Tert-butyl 4-(4-chloro-3-cyclopropylphenyl)piperazine-1-carboxylate

A mixture of 4-bromo-1-chloro-2-cyclopropyl-benzene (1 g, 4 mmol), tert-butyl piperazine-1-carboxylate (965 mg, 5.18 mmol, CAS #57260-71-6), Cs₂CO₃ (4.22 g, 12.9 mmol), RuPhos (202 mg, 432 μmol) and Pd₂(dba)₃ (198 mg, 216 μmol) in dioxane (20 mL) was degassed and purged with N₂ three times, and then the mixture was stirred at 80° C. for 2 hrs under N₂ atmosphere. The reaction mixture was concentrated under reduced pressure to give a residue. The residue was purified by column chromatography (SiO₂, Petroleum ether/Ethyl acetate=50/1 to 10/1) to give the title compound (1.4 g, 83% yield) as a yellow oil. LC-MS (ESI⁺) m/z 336.9 (M+H)⁺.

### Step 3—1-(4-Chloro-3-cyclopropylphenyl)piperazine

To a solution of tert-butyl 4-(4-chloro-3-cyclopropyl-phenyl)piperazine-1-carboxylate (1.4 g, 4.2 mmol) in DCM (15 mL) was added TFA (4.67 g, 40.9 mmol). The mixture was then stirred at 25° C. for 1 hr. On completion, the reaction mixture was concentrated under reduced pressure to give a residue. The crude product was purified by reverse phase HPLC (0.1% NH₄HCO₃ condition) to give the title compound (980 mg, 90% yield) as a brown oil. LC-MS (ESI⁺) m/z 236.9 (M+H)⁺; ¹H NMR (400 MHz, CHLOROFORM-d) δ=9.78 (d, J=1.2 Hz, 1H), 7.27-7.25 (m, 1H), 6.72-6.63 (m, 1H), 6.51 (s, 1H), 3.34 (s, 8H), 2.22-2.11 (m, 1H), 1.10-0.93 (m, 2H), 0.66 (q, J=5.2 Hz, 2H).

### 4-(4-(4-Chloro-3-cyclopropylphenyl)piperazin-1-yl)-5-fluoro-2-methoxyaniline (Intermediate GT)

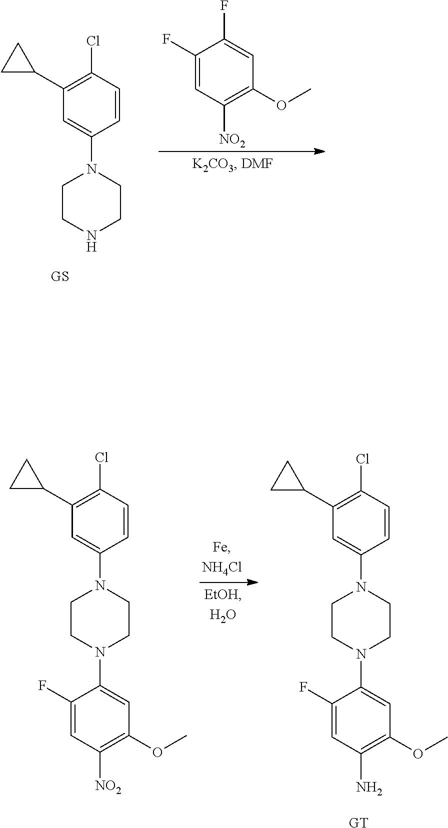

### Step 1—1-(4-chloro-3-cyclopropylphenyl)-4-(2-fluoro-5-methoxy-4-nitrophenyl)piperazine

To a solution of 1-(4-chloro-3-cyclopropyl-phenyl)piperazine (0.98 g, 4.14 mmol, Intermediate GS) in DMF (20 mL) was added K₂CO₃ (1.72 g, 12.4 mmol) and 1,2-difluoro-4-methoxy-5-nitro-benzene (783 mg, 4.14 mmol). Then the mixture was stirred at 80° C. for 2 hrs. On completion, the crude product was triturated with H₂O (50 mL) at 0° C. for 10 mins to give the title compound (1.7 g, 94% yield) as a brown solid. LC-MS (ESI⁺) m/z 405.8 (M+H)⁺.

### Step 2—4-(4-(4-Chloro-3-cyclopropylphenyl)piperazin-1-yl)-5-fluoro-2-methoxyaniline

To a solution of 1-(4-chloro-3-cyclopropyl-phenyl)-4-(2-fluoro-5-methoxy-4-nitro-phenyl)piperazine (1.7 g, 4.19 mmol) in EtOH (20 mL) and H₂O (5 mL) was added NH₄Cl (2.24 g, 41.9 mmol) and Fe (2.34 g, 41.9 mmol) dropwise at 0° C. The mixture was then stirred at 80° C. for 2 hrs. On completion, the reaction mixture was filtered and concentrated under reduced pressure to give the title compound (1.4 g) as a brown solid. LC-MS (ESI⁺) m/z 375.9 (M+H)⁺.

### 3-((4-(4-(4-Chloro-3-cyclopropylphenyl)piperazin-1-yl)-5-fluoro-2-methoxyphenyl)amino)piperidine-2,6-dione (Intermediate GU)

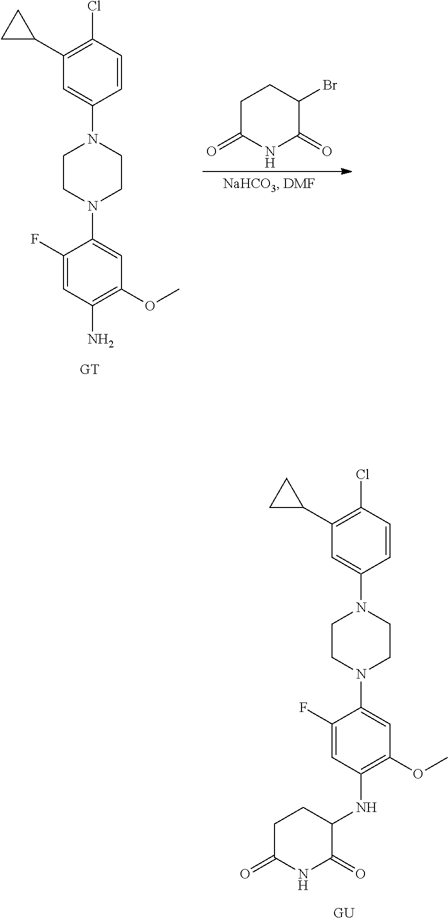 [SMILES: COc1cc(N2CCN(c3ccc(Cl)c(C4CC4)c3)CC2)c(F)cc1N ; O=C1CCC(Br)C(=O)N1 ; COc1cc(N2CCN(c3ccc(Cl)c(C4CC4)c3)CC2)c(F)cc1NC1CCC(=O)NC1=O]

To a solution of 4-[4-(4-chloro-3-cyclopropyl-phenyl)piperazin-1-yl]-5-fluoro-2-methoxy-aniline (1.4 g, 3.7 mmol, Intermediate GT) in DMF (10 mL) was added NaHCO₃ (939 mg, 11.2 mmol) and 3-bromopiperidine-2,6-dione (2.15 g, 11.17 mmol, CAS #62595-74-8). Then the mixture was stirred at 80° C. for 12 hrs. On completion, the reaction mixture was filtered and concentrated under reduced pressure to give a residue. The crude product was purified by reverse phase HPLC (0.1% NH₄HCO₃ condition) to give the title compound (1.1 g, 60% yield) as a brown solid. LC-MS (ESI⁺) m/z 486.9 (M+H)⁺; ¹H NMR (400 MHz, DMSO-d₆) δ=10.84 (s, 1H), 7.22 (d, J=8.8 Hz, 1H), 6.80 (dd, J=2.8, 8.8 Hz, 1H), 6.64 (d, J=8.0 Hz, 1H), 6.57 (d, J=14.4 Hz, 1H), 6.53 (d, J=2.8 Hz, 1H), 5.12 (d, J=6.8 Hz, 1H), 4.34-4.20 (m, 1H), 3.80 (s, 3H), 3.26-3.21 (m, 4H), 3.06-2.99 (m, 4H), 2.80 (ddd, J=5.2, 13.2, 18.0 Hz, 1H), 2.56 (d, J=3.2 Hz, 1H), 2.19-2.03 (m, 2H), 1.92 (dq, J=4.4, 12.8 Hz, 1H), 0.99-0.93 (m, 2H), 0.75-0.69 (m, 2H).

### Tert-butyl (S)-4-(4-chloro-3-fluorophenyl)-2-methylpiperazine-1-carboxylate (Intermediate GV)

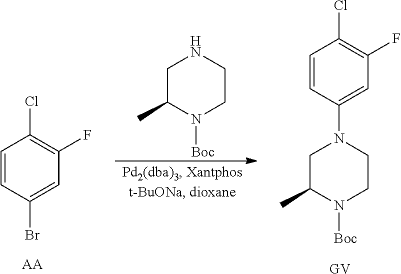

To a solution of 4-bromo-1-chloro-2-fluorobenzene (2.00 g, 9.55 mmol, CAS #60811-18-9), tert-butyl (S)-2-methylpiperazine-1-carboxylate (2.10 g, 10.5 mmol, CAS #170033-47-3) in dioxane (20.0 mL) was added Pd₂(dba)₃ (437 mg, 477 μmol), Xantphos (607 mg, 1.05 mmol) and tBuONa (2.75 g, 28.6 mmol). Then the reaction was stirred at 100° C. for 1 hr under nitrogen atmosphere. On completion, the reaction mixture was diluted with water (20.0 mL) and extracted with ethyl acetate (3×100 mL). The combined organic layers were washed with brine (20.0 mL) and dried over anhydrous sodium sulfate, filtered and the filtrate was concentrated in vacuo to give the crude residue. The crude residue was purified by column chromatography (SiO₂, Petroleum ether/Ethyl acetate=1/0 to 10/1) to give the title compound (2.70 g, 83% yield) as a white solid. LC-MS (ESI⁺) m/z 273.2 (M−55)⁺.

### (S)-7-fluoro-4-(2-fluoro-4-(3-methylpiperazin-1-yl)phenyl)-6-(1-isobutyryl-1,2,5,6-tetrahydropyridin-3-yl)-N,N-dimethyl-1H-indole-2-carboxamide (Intermediate GW)

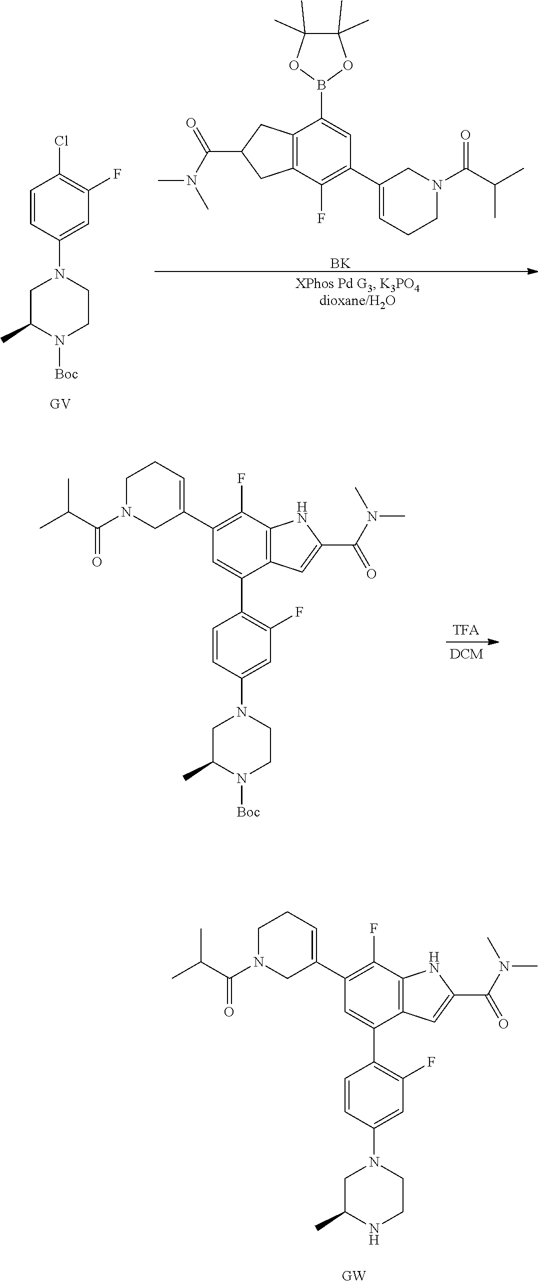

### Step 1—Tert-butyl (S)-4-(4-(2-(dimethylcarbamoyl)-7-fluoro-6-(1-isobutyryl-1,2,5,6-tetrahydropyridin-3-yl)-1H-indol-4-yl)-3-fluorophenyl)-2-methylpiperazine-1-carboxylate

To a solution of tert-butyl (S)-4-(4-chloro-3-fluorophenyl)-2-methylpiperazine-1-carboxylate (500 mg, 1.52 mmol, Intermediate GV), 7-fluoro-6-(1-isobutyryl-1,2,5,6-tetrahydropyridin-3-yl)-N,N-dimethyl-4-(4,4,5,5-tetramethyl-1,3,2-dioxaborolan-2-yl)-1H-indole-2-carboxamide (808 mg, 1.67 mmol, Intermediate BK) in dioxane (6.00 mL) and H₂O (3.00 mL) was added K₃PO₄ (968 mg, 4.56 mmol) and XPhos Pd G3 (128 mg, 152 μmol). Then the reaction was stirred at 80° C. for 1 hr under nitrogen atmosphere. On completion, the reaction mixture was diluted with water (10.0 mL) and extracted with ethyl acetate (3×50.0 mL). The combined organic layers were washed with brine (10.0 mL) and dried over anhydrous sodium sulfate, filtered and the filtrate was concentrated in vacuo to give the crude residue. The crude residue was purified by column chromatography (SiO₂, Petroleum ether/Ethyl acetate=10/0 to 5/1) to give the title compound (900 mg, 81% yield) as a yellow solid. LC-MS (ESI⁺) m/z 650.5 (M+H)⁺.

### Step 2—(S)-7-fluoro-4-(2-fluoro-4-(3-methylpiperazin-1-yl)phenyl)-6-(1-isobutyryl-1,2,5,6-tetrahydropyridin-3-yl)-N,N-dimethyl-1H-indole-2-carboxamide

To a solution of tert-butyl (S)-4-(4-(2-(dimethylcarbamoyl)-7-fluoro-6-(1-isobutyryl-1,2,5,6-tetrahydropyridin-3-yl)-1H-indol-4-yl)-3-fluorophenyl)-2-methylpiperazine-1-carboxylate (900 mg, 1.39 mmol) in DCM (8.00 mL) was added TFA (3.07 g, 26.9 mmol, 2.00 mL). The mixture was stirred at 25° C. for 1 hr. On completion, the reaction mixture was concentrated in vacuo to give the crude residue. The crude residue was purified by reverse phase HPLC (0.8 g/L ammonium bicarbonate) to give the title compound (500 mg, 66% yield) as a white solid. LC-MS (ESI⁺) m/z 550.0 (M+H)⁺.

### 6-(1-(3-(1H-1,2,3-triazol-1-yl)propanoyl)-1,2,5,6-tetrahydropyridin-3-yl)-4-chloro-N-cyclopropyl-7-fluoro-N-methyl-1H-indole-2-carboxamide (Intermediate GX)

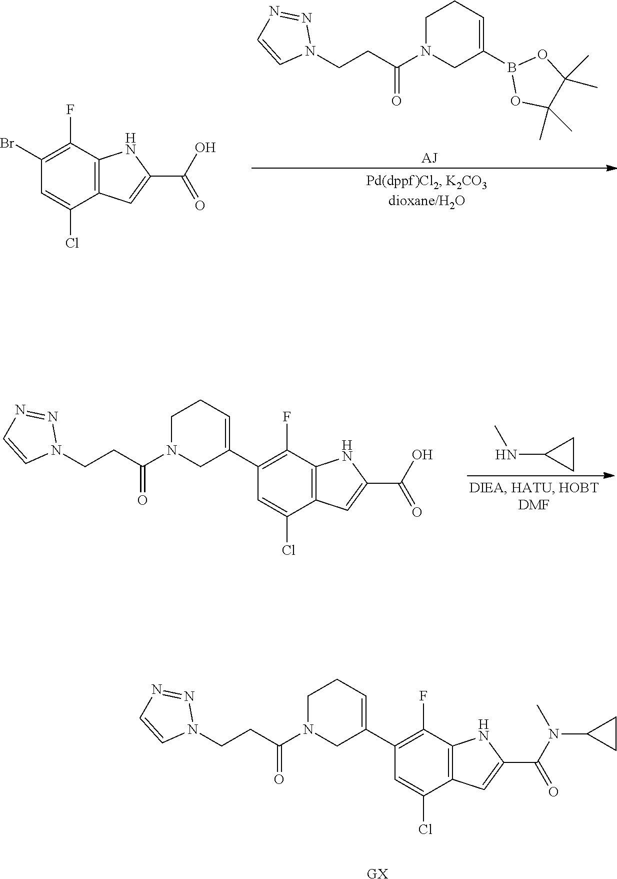

### Step 1—6-(1-(3-(1H-1,2,3-triazol-1-yl)propanoyl)-1,2,5,6-tetrahydropyridin-3-yl)-4-chloro-7-fluoro-1H-indole-2-carboxylic acid

A mixture of 6-bromo-4-chloro-7-fluoro-1H-indole-2-carboxylic acid (1 g, 3 mmol, synthesized via Step 1 of Intermediate T), 1-(5-(4,4,5,5-tetramethyl-1,3,2-dioxaborolan-2-yl)-3,6-dihydropyridin-1 (2H)-yl)-3-(1H-1,2,3-triazol-1-yl)propan-1-one (1.14 g. 3.42 mmol, Intermediate AJ), Pd(dppf)Cl₂ (250 mg, 341 μmol), and K₂CO₃ (1.42 g, 10.3 mmol) in dioxane (10 mL) and H₂O (1 mL) was degassed and purged with N₂ three times. Then the mixture was stirred at 80° C. for 2 hr under N₂ atmosphere. On completion, the reaction mixture was added HCl (1N) until the pH=7, then diluted with water (30 mL) and extracted with dichloromethane (3×30 mL). The combined organic layers were dried over anhydrous sodium sulfate, filtered and the filtrate was concentrated to give the title compound (900 mg) as a yellow solid. LC-MS (ESI⁺) m/z 418.0 (M+H)⁺.

### Step 2—6-(1-(3-(1H-1,2,3-triazol-1-yl)propanoyl)-1,2,5,6-tetrahydropyridin-3-yl)-4-chloro-N-cyclopropyl-7-fluoro-N-methyl-1H-indole-2-carboxamide

To a solution of 6-(1-(3-(1H-1,2,3-triazol-1-yl)propanoyl)-1,2,5,6-tetrahydropyridin-3-yl)-4-chloro-7-fluoro-1H-indole-2-carboxylic acid (850 mg, 2.03 mmol) in DMF (20 mL) was added HATU (928 mg, 2.44 mmol), DIEA (1.31 g, 10.1 mmol, 1.77 mL), HOBt (412 mg, 3.05 mmol), and N-methylcyclopropanamine (144 mg, 2.03 mmol, CAS #5163-20-2). Then the mixture was stirred at 25° C. for 2 hr. On completion, the reaction mixture was filtered and the filter cake was dried in vacuo to give the title compound (400 mg) as a white solid. LC-MS (ESI⁺) m/z 471.1 (M+H)⁺.

### 1-(5-(4-Chloro-7-fluoro-1H-indazol-6-yl)-3,6-dihydropyridin-1 (2H)-yl)-2-methylpropan-1-one (Intermediate GY)

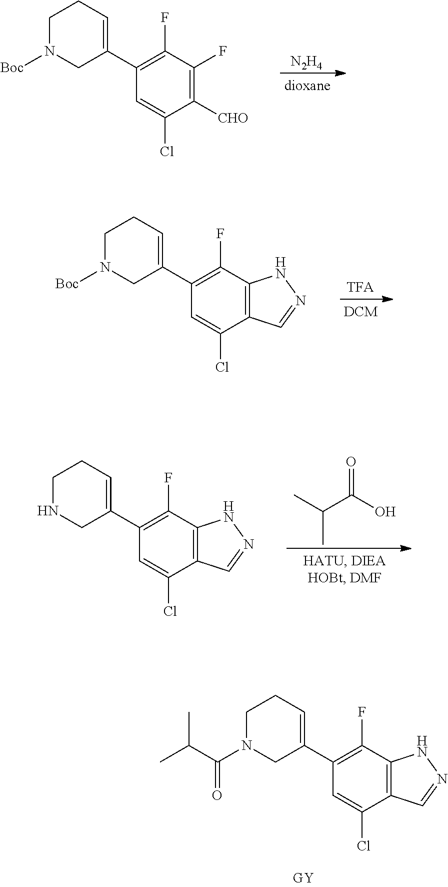

### Step 1—Tert-butyl 5-(4-chloro-7-fluoro-1H-indazol-6-yl)-3,6-dihydropyridine-1 (2H)-carboxylate

To a solution of tert-butyl 5-(5-chloro-2,3-difluoro-4-formyl-phenyl)-3,6-dihydro-2H-pyridine-1-carboxylate (3 g, 8.39 mmol, synthesized via Step 1—2 of Intermediate BY) in dioxane (30 mL) was added hydrazine (948 mg, 25.1 mmol, 1.07 mL). The mixture was then stirred at 80° C. for 48 hr. On completion, the reaction mixture was quenched by adding H₂O (50 mL) at 25° C., then filtered to obtain the crude reaction solution. The solution was diluted with EA (50 mL), and extracted with EA (50 mL×3). The combined organic layer was washed with 20 mL brine and dried with Na₂SO₄. Filtered and the filtrate was concentrated to give a crude residue. The residue was purified by column chromatography (SiO₂, Petroleum ether/Ethyl acetate=100:0 to 3:1) to give the title compound (2.8 g, 91% yield) as a yellow solid. LC-MS (ESI⁺) m/z 296.1 (M−55)⁺.

### Step 2—4-Chloro-7-fluoro-6-(1,2,5,6-tetrahydropyridin-3-yl)-1H-indazole

To a solution of tert-butyl 5-(4-chloro-7-fluoro-1H-indazol-6-yl)-3,6-dihydro-2H-pyridine-1-carboxylate (2.8 g, 8.0 mmol) in DCM (15 mL) was added TFA (5 mL). The mixture was stirred at 25° C. for 1 hr. On completion, the reaction mixture was concentrated under reduced pressure to give a residue. The crude product was purified by reverse phase HPLC (0.1% NH₄HCO₃ condition) to give the title compound (1.8 g, 85% yield) as a white solid. LC-MS (ESI⁺) m/z 252.1 (M+H)⁺.

### Step 3—1-(5-(4-Chloro-7-fluoro-1H-indazol-6-yl)-3,6-dihydropyridin-1 (2H)-yl)-2-methylpropan-1-one

To a solution of 2-methylpropanoic acid (210 mg, 2.38 mmol, CAS #79-31-2) in DMF (2 mL) was added HATU (664 mg, 1.75 mmol), HOBt (429 mg, 3.18 mmol) and DIEA (2.05 g, 15.8 mmol). Then 4-chloro-7-fluoro-6-(1,2,3,6-tetrahydropyridin-5-yl)-1H-indazole (400 mg, 1.59 mmol) was added at 0° C. The mixture was then stirred at 25° C. for 1 hr. On completion, the reaction mixture was quenched by addition of water (5 mL) at 0° C., and extracted by ethyl acetate (3×20 mL). The combined organic layers were dried over anhydrous sodium sulfate, filtered and the filtrate was concentrated in vacuo to give the title compound (400 mg) as a brown gum, LC-MS (ESI⁺) m/z 322.0 (M+H)⁺.

### 3-[4-[4-[3-methoxy-4-(4,4,5,5-tetramethyl-1,3,2-dioxaborolan-2-yl)phenyl]-1-piperidyl]-3-methyl-2-oxo-benzimidazol-1-yl]piperidine-2,6-dione (Intermediate GZ)

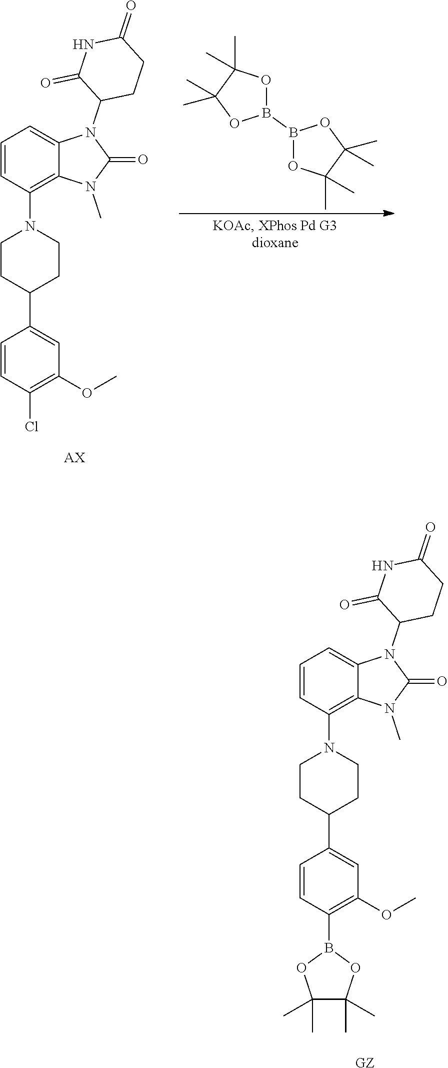

A mixture of 3-[4-[4-(4-chloro-3-methoxy-phenyl)-1-piperidyl]-3-methyl-2-oxo-benzimidazol-1-yl]piperidine-2,6-dione (180 mg, 373 μmol, Intermediate AX), 4,4,5,5-tetramethyl-2-(4,4,5,5-tetramethyl-1,3,2-dioxaborolan-2-yl)-1,3,2-dioxaborolane (379 mg, 1.49 mmol, CAS #73183-34-3), XPhos Pd G3 (31.6 mg, 37.3 μmol) and KOAc (110 mg, 1.12 mmol) in dioxane (5 mL) was degassed and purged with N₂ three times. Then the mixture was stirred at 80° C. for 2 hrs under N₂ atmosphere. On completion, the reaction mixture was concentrated under reduced pressure to give a residue. The residue was purified by column chromatography (SiO₂, Petroleum ether/Ethyl acetate=1/0 to 5/1) to give the title compound (220 mg, 82% yield) as a white solid. LC-MS (ESI⁺) m/z 575.2 (M+H)⁺.

### 4-(4-Chloro-3-(trifluoromethoxy)phenyl)piperidine (Intermediate HA)

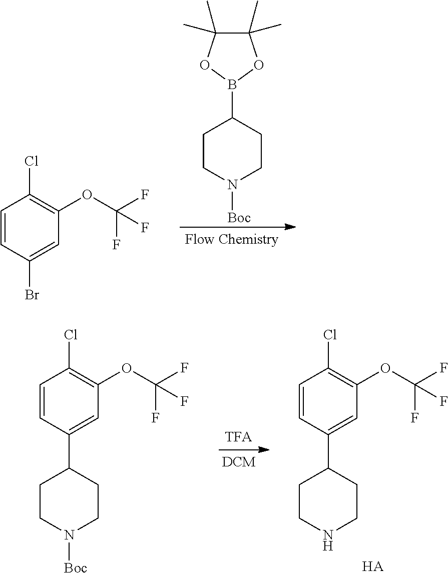

### Step 1—Tert-butyl 4-(4-chloro-3-(trifluoromethoxy)phenyl)piperidine-1-carboxylate

To a 250 mL vial equipped with a stir bar was added 4-bromo-1-chloro-2-(trifluoromethoxy)benzene (3.5 g, 12.7 mmol, CAS #406230-79-9), tert-butyl 4-(4,4,5,5-tetramethyl-1,3,2-dioxaborolan-2-yl)piperidine-1-carboxylate (3.95 g. 12.7 mmol. CAS #1048970-17-7), bis[3,5-difluoro-2-[5-(trifluoromethyl)-2-pyridyl]phenyl]iridium (1+) 4-tert-butyl-2-(4-tert-butyl-2-pyridyl)pyridine; hexafluorophosphate (142 mg, 127 μmol, CAS #870987-63-6), 4-tert-butyl-2-(4-tert-butyl-2-pyridyl)pyridine dichloronickel (252 mg, 635 μmol, CAS #1034901-50-2) and morpholine (1.66 g, 19.0 mmol, 1.68 mL, CAS #110-91-8) in dry DMF (150 mL). The vial was sealed and placed under air. The reaction solution was pumped through the reactor at a flow rate of 300 μL min-1 (residence time of 26.67 min), irradiating with a 450 nm LED lamp, with cooling fan to keep the reaction temperature at 25° C. On completion, the reaction mixture was diluted with water (500 mL) and extracted with ethyl acetate (2×100 mL). The combined organic layers were washed with brine (200 mL) and dried over anhydrous sodium sulfate, filtered and the filtrate was concentrated in vacuo to give the crude residue. The crude residue was purified by reverse phase HPLC (0.1% FA condition) to give the title compound (2.9 g, 60% yield) as a yellow oil. LC-MS (ESI⁺) m/z 323.7 (M−55)⁺.

### Step 2—4-(4-Chloro-3-(trifluoromethoxy)phenyl)piperidine

To a solution of tert-butyl 4-(4-chloro-3-(trifluoromethoxy)phenyl)piperidine-1-carboxylate (500 mg, 1.32 mmol) in DCM (5 mL) was added TFA (1.54 g, 1 mL). The mixture was then stirred at 25° C. for 1 hr. On completion, the mixture was concentrated under reduced pressure to give the title compound (400 mg) as yellow oil. LC-MS (ESI⁺) m/z 280.1 (M+H)⁺.

### Tert-butyl 3-(4-chlorophenyl)azetidine-1-carboxylate (Intermediate HB)

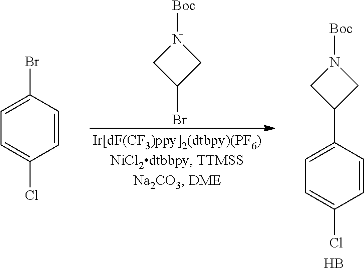

To a solution of 1-bromo-4-chlorobenzene (3.00 g, 15.8 mmol, CAS #106-39-8) and tert-butyl 3-bromoazetidine-1-carboxylate (4.81 g, 20.4 mmol, CAS #1064194-10-0) in DME (60.0 mL) was added Ir[dF(CF₃)ppy]₂(dtbpy)(PF₆) (176 mg, 157 μmol), NiCl₂·dtbbpy (93.6 mg, 235 μmol), TTMSS (3.90 g, 15.7 mmol) and Na₂CO₃ (3.32 g, 31.3 mmol). Then the mixture was stirred and irradiated with a 10 W blue LED lamp (3 cm away) under N₂ atmosphere, with cooling water to keep the reaction temperature at 25° C. for 14 h. On completion, the reaction mixture was quenched by addition of NH₄Cl (500 mL) at 0° C., and then extracted with EA (100 mL×3). The combined organic layers were washed with brine (200 mL×2), dried over Na₂SO₄, filtered and concentrated under reduced pressure to give a residue. The residue was purified by flash silica gel chromatography (ISCO®; 40 g SepaFlash® Silica Flash Column, Eluent of 0˜15% Ethyl acetate/Petroleum ether gradient a 30 mL/min) to give the title compound (3.5 g, 74% yield) as a colorless oil. LC-MS (ESI⁺) m/z 212.1 (M+H)⁺.

### 5-(1-(3-(1H-pyrazol-1-yl)propanoyl)-1,2,5,6-tetrahydropyridin-3-yl)-4-fluoro-N,N-dimethyl-7-(4,4,5,5-tetramethyl-1,3,2-dioxaborolan-2-yl)benzofuran-2-carboxamide (Intermediate HC)

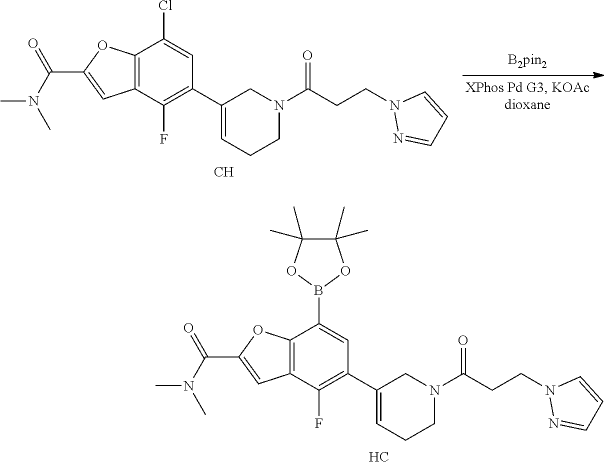

To a solution of 5-(1-(3-(1H-pyrazol-1-yl)propanoyl)-1,2,5,6-tetrahydropyridin-3-yl)-7-chloro-4-fluoro-N,N-dimethylbenzofuran-2-carboxamide (3 g, 6.74 mmol, Intermediate CH) and 4,4,5,5-tetramethyl-2-(4,4,5,5-tetramethyl-1,3,2-dioxaborolan-2-yl)-1,3,2-dioxaborolane (8.56 g, 33.7 mmol) in dioxane (30 mL) was added KOAc (1.99 g, 20.2 mmol) and XPhos Pd G3 (570 mg, 674 μmol), then the mixture was stirred at 60° C. for 1 h under N₂. On completion, the reaction mixture was diluted with water (200 mL) and extracted with EtOAc (50 mL×2). The combined organic layers were washed with brine (50 mL×2), dried over Na₂SO₄, filtered and concentrated under reduced pressure to give a residue. The residue was purified by flash silica gel chromatography (ISCO®; 20 g SepaFlash® Silica Flash Column, Eluent of 30˜100% Ethyl acetate/Petroleum ether gradient @ 30 mL/min) to give the title compound (3.2 g, 80% yield) as a white solid. LC-MS (ESI⁺) m/z 537.2 (M+H)⁺.

### 5-(1-(3-(1H-pyrazol-1-yl)propanoyl)-1,2,5,6-tetrahydropyridin-3-yl)-7-(4-(azetidin-3-yl)phenyl)-4-fluoro-N,N-dimethylbenzofuran-2-carboxamide (Intermediate HD)

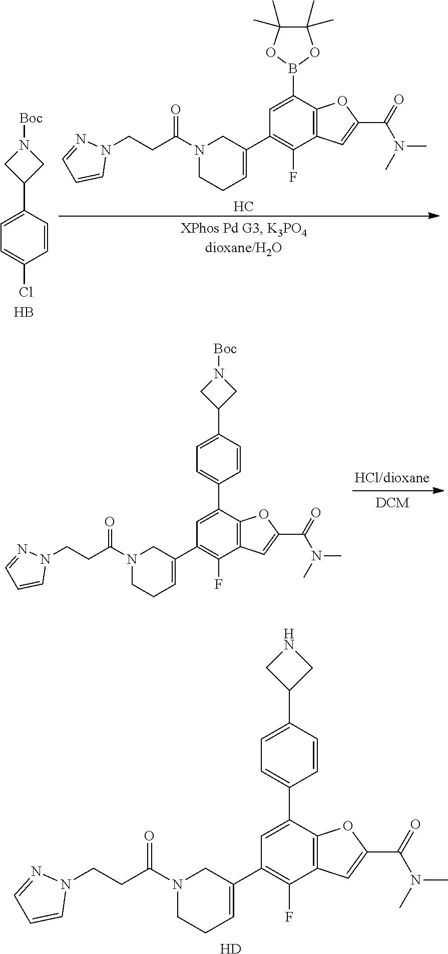

### Step 1—tert-butyl 3-(4-(5-(1-(3-(1H-pyrazol-1-yl)propanoyl)-1,2,5,6-tetrahydropyridin-3-yl)-2-(dimethylcarbamoyl)-4-fluorobenzofuran-7-yl)phenyl)azetidine-1-carboxylate

To a solution of tert-butyl 3-(4-chlorophenyl)azetidine-1-carboxylate (99.8 mg, 373 μmol, Intermediate HB) and 5-(1-(3-(1H-pyrazol-1-yl)propanoyl)-1,2,5,6-tetrahydropyridin-3-yl)-4-fluoro-N,N-dimethyl-7-(4,4,5,5-tetramethyl-1,3,2-dioxaborolan-2-yl)benzofuran-2-carboxamide (200 mg, 373 μmol, Intermediate HC) in dioxane (1.00 mL) and H₂O (0.10 mL) was added XPhos Pd G3 (32.0 mg, 37.3 μmol) and K₃PO₄ (237 mg, 1.12 mmol). Then the mixture was stirred at 80° C. for 2 h under N₂ atmosphere. On completion, the reaction mixture was then diluted with water (20 mL) and extracted with EA (10 mL×3). The combined organic layers were washed with brine (10 mL×2), dried over Na₂SO₄, filtered and concentrated under reduced pressure to give a residue. The residue was purified by flash silica gel chromatography (ISCO®; 4 g SepaFlash® Silica Flash Column, Eluent of 0-50% Ethyl acetate/Petroleum ether gradient (10 mL/min) to give the title compound (130 mg, 46% yield) as a yellow oil. LC-MS (ESI⁺) m/z 642.2 (M+H)⁺.

### Step 2—5-(1-(3-(1H-pyrazol-1-yl)propanoyl)-1,2,5,6-tetrahydropyridin-3-yl)-7-(4-(azetidin-3-yl)phenyl)-4-fluoro-N,N-dimethylbenzofuran-2-carboxamide

To a solution of tert-butyl 3-(4-(5-(1-(3-(1H-pyrazol-1-yl)propanoyl)-1,2,5,6-tetrahydropyridin-3-yl)-2-(dimethylcarbamoyl)-4-fluorobenzofuran-7-yl)phenyl)azetidine-1-carboxylate (130 mg, 203 μmol) in DCM (1.00 mL) was added HCl/dioxane (4 M, 910 L), then the mixture was stirred at 20° C. for 1 hr. On completion, the reaction mixture was filtered and concentrated under reduced pressure to give the title compound (150 mg, HCl) as a yellow gum, LC-MS (ESI⁺) m/z 542.2 (M+H)⁺.

### 4-Chloro-3-(2,6-dioxopiperidin-3-yl)benzoic acid (Intermediate HE)

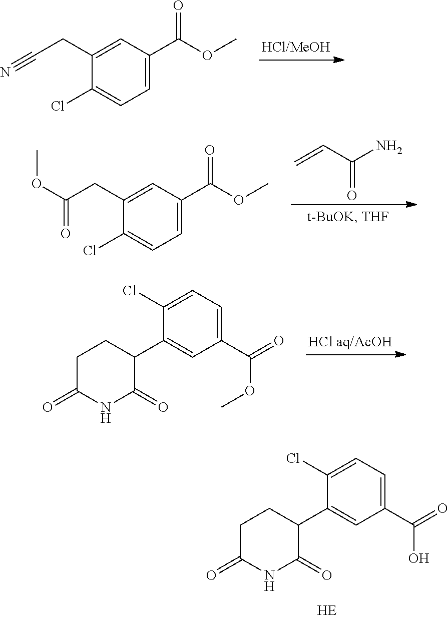

### Step 1—Methyl 4-chloro-3-(2-methoxy-2-oxoethyl)benzoate

A solution of methyl 4-chloro-3-(cyanomethyl)benzoate (10.0 g, 47.7 mmol, CAS #872091-83-3) in HCl/MeOH (4 M, 100 mL) was stirred at 70° C. for 12 hr. On completion, the reaction mixture was concentrated to give the crude product. The residue was diluted with EA (100 mL), filtered and the filtrate was concentrated to give the title compound (12.0 g) as a colorless oil. ¹H NMR (400 MHz, CDCl₃) δ ppm 7.98 (d, J=2.0 Hz, 1H), 7.91 (dd, J=2.0, 8.4 Hz, 1H), 7.47 (d, J=8.4 Hz, 1H), 3.92 (s, 3H), 3.83 (s, 2H), 3.73 (s, 3H).

### Step 2—Methyl 4-chloro-3-(2,6-dioxopiperidin-3-yl)benzoate

To a mixture of methyl 4-chloro-3-(2-methoxy-2-oxoethyl)benzoate (9.76 g, 40.2 mmol) and acrylamide (2.86 g, 40.2 mmol, 2.78 mL, CAS #9003-05-8) in THF (100 mL) was added t-BuOK (1.0 M, 44.2 mL) at 0° C. under N₂ atmosphere. Then the mixture was stirred at 50° C. for 2 hr. On completion, the mixture was added to aqueous NH₄Cl (400 mL) at 0° C. slowly, then extracted with EA (60.0 mL×3). The combined organic layers were concentrated under reduced pressure to give a residue and it was triturated with EA (30.0 mL) at 25° C. for 10 min, then filtered and the filter cake was dried over under reduced pressure to give the title compound (7.37 g, 59% yield) as a white solid. LC-MS (ESI⁺) m/z 282.1 (M+H)⁺; ¹H NMR (400 MHz, DMSO-d₆) δ ppm 10.95 (s, 1H), 7.93 (d, J=2.0 Hz, 1H), 7.87 (dd, J=2.4, 8.4 Hz, 1H), 7.64 (d, J=8.0 Hz, 1H), 4.40-4.29 (m, 1H), 3.86 (s, 3H), 2.87-2.71 (m, 1H), 2.40-2.28 (m, 2H), 2.04-1.97 (in, 1H).

### Step 3—4-Chloro-3-(2,6-dioxopiperidin-3-yl)benzoic acid

A mixture of methyl 4-chloro-3-(2,6-dioxopiperidin-3-yl)benzoate (3.0 g, 10.7 mmol) in HCl (30.0 mL) and AcOH (30.0 mL) was stirred at 60° C. for 2.5 hr. On completion, the mixture was concentrated, then adjusted to pH=4-5 by addition of aq. NaHCO₃ at 0° C. Then the mixture was concentrated and diluted with DMSO (20.0 mL) to give residue solution. The solution was purified by prep-HPLC (column: Phenomenex luna C18 (250*70 mm, 10 um); mobile phase: [water (FA)-ACN]; gradient: 12%-42% B over 15 min) then triturated with ACN (10.0 mL) at 20° C. for 10 min. The mixture was filtered and the filter cake was dissolved with DMSO (2.0 mL), added to water (40.0 mL), filtered and the filter cake was dried over under reduced pressure to give the title compound (1.04 g, 34% yield) as a white solid. LC-MS (ESI⁺) m/z 268.1 (M+H)⁺: ¹H NMR (400 MHz, DMSO-d₆) δ ppm 13.72-12.47 (m, 1H), 10.95 (s, 1H), 7.89 (d, J=1.6 Hz, 1H), 7.88-7.83 (m, 1H), 7.60 (d, J=8.4 Hz, 1H), 4.31 (dd, J=4.4, 12.4 Hz, 1H), 2.86-2.70 (m, 1H), 2.34 (dq, J=4.4, 12.8 Hz, 2H), 2.06-1.94 (m, 1H).

### 4-Bromo-1-(4-chlorophenyl)piperidine (Intermediate HF)

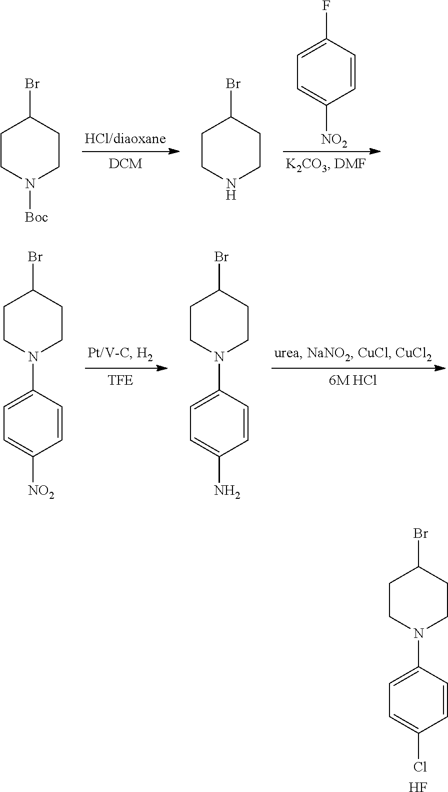

### Step 1—4-Bromopiperidine

To a solution of tert-butyl 4-bromopiperidine-1-carboxylate (10 g, 37.8 mmol, CAS #180695-79-8) in DCM (100 mL) was added HCl/dioxane (1.5 M, 25.2 mL). The mixture was stirred at 40° C. for 4 hrs. On completion, the reaction mixture was filtered and concentrated under reduced pressure to give the title compound (15 g) as a white solid. ¹H NMR (400 MHz, DMSO-d₆) δ=4.62-4.45 (m, 1H), 3.25-3.11 (m, 2H), 3.09-2.95 (m, 2H), 2.29-2.22 (m, 2H), 2.07-1.94 (in, 2H).

### Step 2—4-Bromopiperidine

A mixture of 4-bromopiperidine (15 g, 91.4 mmol), 1-fluoro-4-nitro-benzene (11.7 g, 83.1 mmol, 8.82 mL, CAS #350-46-9), and K₂CO₃ (34.5 g, 249 mmol) in DMF (150 mL) was degassed and purged with N₂ three times, and then the mixture was stirred at 60° C. for 2 hrs under N₂ atmosphere. On completion, the reaction mixture was filtered and concentrated under reduced pressure to give the title compound (17 g) as a white solid. LC-MS (ESI⁺) m/z 285.0 (M+H)⁺.

### Step 3—4-(4-Bromopiperidin-1-yl)aniline

A mixture of 4-bromo-1-(4-nitrophenyl) piperidine (2 g, 7 mmol) and Pt/V-C (1 g, 7 mmol) in THF (20 mL) was degassed and purged with H₂ three times. Then the mixture was stirred at 25° C. for 12 hrs under H₂ (15 psi) atmosphere. On completion, the reaction mixture was quenched with water (20 mL) and extracted with dichloromethane (2×20 mL). The residue was purified by column chromatography (SiO₂. Petroleum ether/Ethyl acetate=10/1 to 0/1) to give the title compound (1 g, 50% yield) as a white solid. LC-MS (ESI⁺) m/z 255.04 (M+H)⁺.

### Step 4—4-Bromo-1-(4-chlorophenyl)piperidine

A mixture of 4-(4-bromo-1-piperidyl)aniline (1 g, 3.92 mmol) and NaNO₂ (376 mg, 5.46 mmol) in HCl at −30° C. was stirred for 0.5 hours. Then urea (155 mg, 2.59 mmol, 139 μL), CuCl (566 mg, 5.72 mmol, 136 μL), and CuCl₂ (769 mg, 5.72 mmol) was added and the mixture was degassed and purged with N₂ three times. Then the mixture was stirred at −30-60° C. for 2 hours under N₂ atmosphere. On completion, the reaction mixture was diluted with water (10 mL) and extracted with ethyl dichloromethane (2×20 mL). The combined organic layers were washed with brine (20 mL) and dried over anhydrous sodium sulfate, filtered and the filtrate was concentrated in vacuo to give the crude residue. The residue was purified by column chromatography (SiO₂, Petroleum ether:Ethyl acetate=1/0 to 10/1) to give the title compound (800 mg. 73% yield) as a white solid. LC-MS (ESI⁺) m/z 273.7 (M+H)⁺.

### 2-(5-Fluoro-2-methoxy-4-(piperidin-4-yl)phenyl)isoindoline-1,3-dione (Intermediate HG)

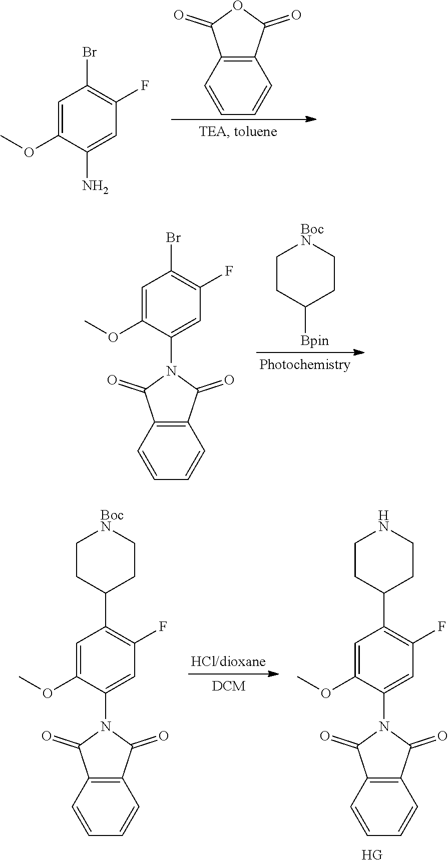

### Step 1—2-(4-Bromo-5-fluoro-2-methoxyphenyl)isoindoline-1,3-dione

To a solution of 4-bromo-5-fluoro-2-methoxyaniline (23.5 g, 106 mmol, CAS #330794-03-1) and isobenzofuran-1,3-dione (15.8 g, 106 mmol, CAS #85-44-9) in toluene (400 mL) was added TEA (10.8 g, 106 mmol, 14.8 mL). The mixture was then stirred at 110° C. for 12 hr. On completion, the reaction mixture was concentrated under reduced pressure to give a crude residue, which was then purified by MPLC (SiO₂. PE:EA=50:1 to 5:1) to give the title compound (25.34 g, 68% yield) as a brown solid. LC-MS (ESI⁺) m/z 350.1 (M+H)⁺.

### Step 2—Tert-butyl 4-(4-(1,3-dioxoisoindolin-2-yl)-2-fluoro-5-methoxyphenyl)piperidine-1-carboxylate

To a solution of 2-(4-bromo-5-fluoro-2-methoxyphenyl)isoindoline-1,3-dione (25.3 g, 72.3 mmol) and tert-butyl 4-(4,4,5,5-tetramethyl-1,3,2-dioxaborolan-2-yl)piperidine-1-carboxylate (27.0 g, 86.8 mmol, CAS #1048970-17-7) in DMF (150 mL) and was added 2,2,3,3,5,5,6,6-octadeuteriomorpholine (9.46 g, 108 mmol), bis[3,5-difluoro-2-[5-(trifluoromethyl)-2-pyridyl]phenyl]iridium (1+); 4-tert-butyl-2-(4-tert-butyl-2-pyridyl)pyridine hexafluorophosphate (811 mg, 723 μmol) and 4-tert-butyl-2-(4-tert-butyl-2-pyridyl)pyridine dichloronickel (1.44 g, 3.62 mmol). Then the mixture was stirred and irradiated with a 455 nm LED lamp (3 cm away) by flow reaction, with cooling water to keep the reaction temperature at 25° C. for 12 h. On completion, the reaction mixture was filtered to give the filtrate as a residue. The residue was purified by reversed-phase HPLC (0.1% FA condition) to give the title compound (22.4 g, 67% yield) as a yellow solid. LC-MS (ESI⁺) m/z 399.1 (M−55)⁺.

### Step 3—2-(5-Fluoro-2-methoxy-4-(piperidin-4-yl)phenyl)isoindoline-1,3-dione

To a solution of tert-butyl 4-(4-(1,3-dioxoisoindolin-2-yl)-2-fluoro-5-methoxyphenyl)piperidine-1-carboxylate (30.9 g, 67.9 mmol) in DCM (300 mL) and was added HCl/dioxane (8 M, 100 mL), then the mixture was stirred at 25° C. for 1 hr. On completion, the reaction mixture was concentrated under reduced pressure to give the title compound (28.6 g, HCl) as a yellow solid. LC-MS (ESI⁺) m/z 355.1 (M+H)⁺.

### 4-Bromo-1-chloro-2-cyclopropylbenzene (Intermediate HH)

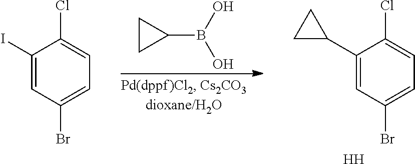

A mixture of 4-bromo-1-chloro-2-iodo-benzene (5.00 g, 15.8 mmol, CAS #774608-49-0), cyclopropylboronic acid (1.35 g, 15.8 mmol, CAS #411235-57-9), Pd(dppf)Cl₂·CH₂Cl₂ (1.29 g, 1.58 mmol), and Cs₂CO₃ (15.4 g, 47.3 mmol) in dioxane (40.0 mL) and H₂O (8.00 mL) was degassed and purged with N₂ three times. Then the mixture was stirred at 100° C. for 12 hrs under N₂ atmosphere. On completion, the reaction mixture was filtered and concentrated under reduced pressure to give a residue. The crude product was purified by MPLC (SiO₂, PE:EA=1:0) to give the title compound (2.40 g, 66% yield) as a colorless oil. ¹H NMR (400 MHz, DMSO-d₆) δ=7.37 (d, J=1.2 Hz, 2H), 7.18 (s, 1H), 2.17-2.06 (m, 1H), 1.04-0.98 (m, 2H), 0.78-0.73 (m, 2H).

### 3-((4-(1-(4-Chloro-3-cyclopropylphenyl)piperidin-4-yl)-5-fluoro-2-methoxyphenyl)amino)piperidine-2,6-dione (Intermediate HI)

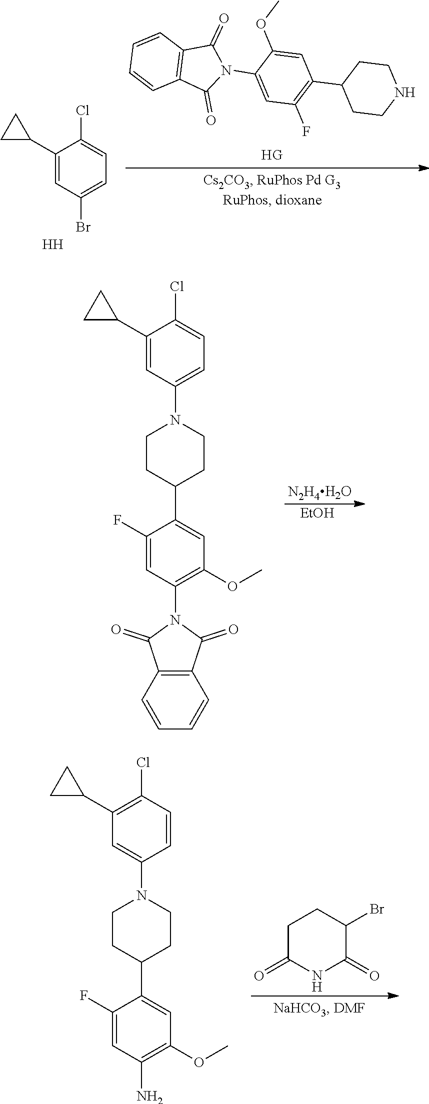

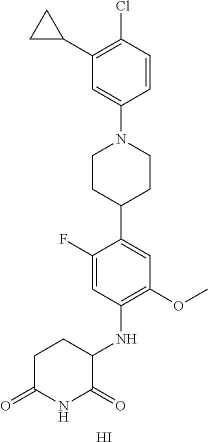

### Step 1—2-(4-(1-(4-Chloro-3-cyclopropylphenyl)piperidin-4-yl)-5-fluoro-2-methoxyphenyl)isoindoline-1,3-dione

To a mixture of 2-[5-fluoro-2-methoxy-4-(4-piperidyl)phenyl]isoindoline-1,3-dione (1.69 g, 4.32 mmol, HCl, Intermediate HG) and 4-bromo-1-chloro-2-cyclopropylbenzene (1.00 g, 4.32 mmol Intermediate HH) in dioxane (20.0 mL) was added RuPhos (202 mg, 0.43 mmol), RuPhos Pd G3 (361 mg, 0.432 mmol) and Cs₂CO₃ (4.22 g, 13.0 mmol). Then the mixture was degassed and purged with N₂ three times, and the mixture was stirred at 100° C. for 1 hr under N₂ atmosphere. On completion, the reaction mixture was filtered and concentrated under reduced pressure to give a residue. The crude product was purified by MPLC(SiO₂, PE:EA=10:1 to 5:1) to give the title compound (660 mg, 24% yield) as a yellow solid. LC-MS (ESI⁺) m/z 505.3 (M+H)⁺.

### Step 2—4-(1-(4-Chloro-3-cyclopropylphenyl)piperidin-4-yl)-5-fluoro-2-methoxyaniline

To a solution of 2-[4-[1-(4-chloro-3-cyclopropyl-phenyl)-4-piperidyl]-5-fluoro-2-methoxy-phenyl]isoindoline-1,3-dione (360 mg, 713 μmol) in EtOH (5.00 mL) and was added N₂H₄·H₂O (437 mg, 7.41 mmol, 423 μL, 85% solution), then the mixture was stirred at 50° C. for 4 hrs. On completion, the reaction mixture was quenched by addition of H₂O (5.00 mL) at 0° C., and then the mixture was extracted with EA (10 mL×2). The combined organic layers were washed with brine (10 mL×3), dried over Na₂SO₄, filtered and the filtrate was concentrated under reduced pressure to give the title compound (510 mg) as a yellow oil. LC-MS (ESI⁺) m/z 375.3 (M+H)⁺.

### Step 3—3-((4-(1-(4-Chloro-3-cyclopropylphenyl)piperidin-4-yl)-5-fluoro-2-methoxyphenyl)amino)piperidine-2,6-dione

To a solution of 4-[1-(4-chloro-3-cyclopropyl-phenyl)-4-piperidyl]-5-fluoro-2-methoxy-aniline (510 mg, 1.36 mmol) and 3-bromopiperidine-2,6-dione (261 mg, 1.36 mmol) in DMF (10.0 mL) was added NaHCO₃ (229 mg, 2.72 mmol), then the mixture was stirred at 80° C. for 12 hrs. On completion, the reaction mixture was filtered and concentrated under reduced pressure to give a residue. The residue was extracted with EA 20 mL (10 mL×2). The combined organic layers were washed with brine (10 mL×3), dried over Na₂SO₄, filtered and concentrated under reduced pressure to give a residue. The crude product was purified by reversed-phase HPLC (0.1% FA condition) to give the title compound (200 mg, 25% yield, FA) as a brown solid. LC-MS (ESI⁺) m/z 486.3 (M+H)⁺.

### 2-Chloro-5-(4-(4-((2,6-dioxopiperidin-3-yl)amino)-2-fluoro-5-methoxyphenyl)piperazin-1-yl)benzonitrile (Intermediate ID)

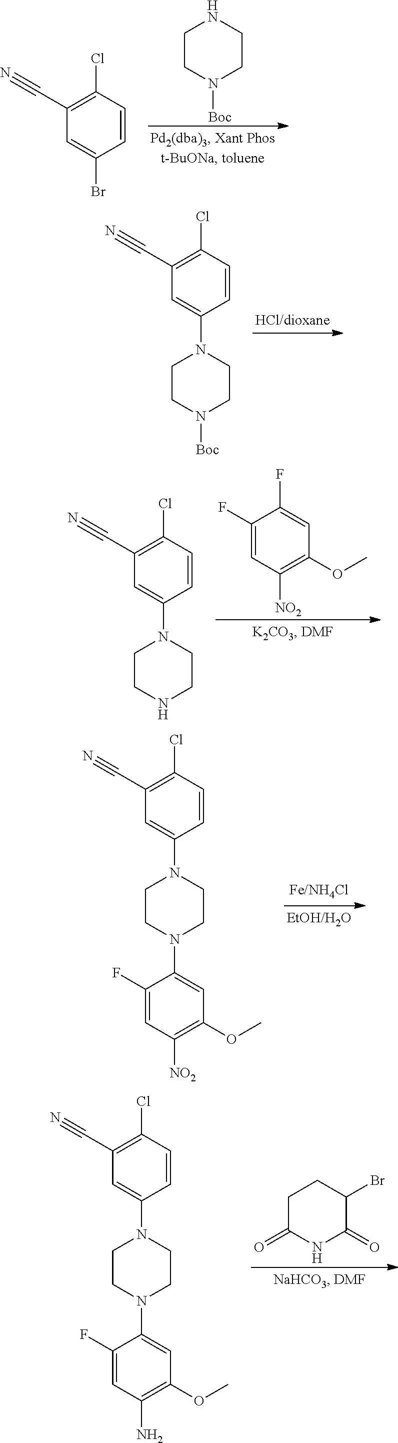

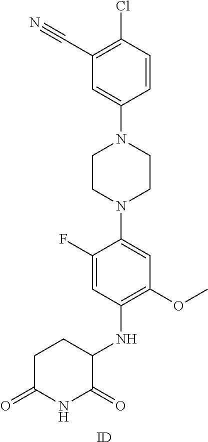

### Step 1—Tert-butyl 4-(4-chloro-3-cyanophenyl)piperazine-1-carboxylate

A mixture of 5-bromo-2-chloro-benzonitrile (5 g, 20 mmol. CAS #57381-44-9), tert-butyl piperazine-1-carboxylate (4.30 g, 23.1 mmol, CAS #57260-71-6), Xantphos (1.60 g, 2.77 mmol), Pd₂(dba)₃ (1.27 g, 1.39 mmol) and t-BuONa (3.11 g, 32.4 mmol) in toluene (100 mL) was degassed and purged with N₂ three times. Then the mixture was stirred at 115° C. for 5 hrs under N₂ atmosphere. On completion, the reaction mixture was concentrated under reduced pressure to give a residue. The residue was purified by column chromatography (SiO₂, Petroleum ether/Ethyl acetate=1/0 to 5/1) to give the title compound (5.77 g, 75% yield) as a yellow solid. LC-MS (ESI⁺) m/z 266.1 (M−55)⁺.

### Step 2—2-Chloro-5-(piperazin-1-yl)benzonitrile

A solution of tert-butyl 4-(4-chloro-3-cyano-phenyl)piperazine-1-carboxylate (2.5 g, 7.77 mmol) in HCl/dioxane (2 M, 20 mL) was stirred at 25° C. for 12 hrs. On completion, the reaction mixture was concentrated under reduced pressure to give the title compound (2.5 g, HCl) as a yellow solid. LC-MS (ESI⁺) m/z 222.0 (M+H)⁺.

### Step 3—2-Chloro-5-(4-(2-fluoro-5-methoxy-4-nitrophenyl)piperazin-1-yl)benzonitrile

To a solution of 2-chloro-5-piperazin-1-yl-benzonitrile (2.5 g, 11.3 mmol) and 1,2-difluoro-4-methoxy-5-nitro-benzene (2.13 g, 11.3 mmol) in DMF (30 mL) was added K₂CO₃ (4.68 g, 33.8 mmol). The mixture was then stirred at 90° C. for 12 hrs. On completion, the reaction mixture was poured into H₂O (150 mL) and stirred for 0.5 hr. The crude product was triturated with H₂O at 25° C. for 0.5 hr and then filtered and dried to give the title compound (1.6 g, 33% yield) as a yellow solid. LC-MS (ESI⁺) m/z 391.0 (M+H)⁺.

### Step 4—5-(4-(4-Amino-2-fluoro-5-methoxyphenyl)piperazin-1-yl)-2-chlorobenzonitrile

To a solution of 2-chloro-5-[4-(2-fluoro-5-methoxy-4-nitro-phenyl)piperazin-1-yl]benzonitrile (1.6 g, 4.09 mmol) in EtOH (15 mL) and H₂O (15 mL) was added Fe (1.60 g, 28.7 mmol) and NH4Cl (1.53 g, 28.7 mmol) under N₂ atmosphere. The mixture was then stirred at 80° C. for 2 hrs. On completion, the reaction mixture was filtered to give the filtrate. The filtrate was diluted with H₂O (50 mL), then NH₃·H₂O was added to adjust the pH to 8, and extracted with EA (70 mL×3). The combined organic layers were dried over Na₂SO₄, filtered and concentrated under reduced pressure to give the title compound (1.5 g) as a purple solid. LC-MS (ESI⁺) m, z 360.9 (M+H)⁺.

### Step 5—2-Chloro-5-(4-(4-((2,6-dioxopiperidin-3-yl)amino)-2-fluoro-5-methoxyphenyl)piperazin-1-yl)benzonitrile

To a solution of 5-[4-(4-amino-2-fluoro-5-methoxy-phenyl)piperazin-1-yl]-2-chloro-benzonitrile (800 mg, 2.22 mmol) in DMF (15 mL) was added NaHCO₃ (745 mg, 8.87 mmol) and 3-bromopiperidine-2,6-dione (1.70 g, 8.87 mmol, CAS #62595-74-8). The mixture was then stirred at 80° C. for 12 hrs. On completion, the reaction mixture was diluted with H₂O (40 mL) and extracted with EA (80 mL×3). The combined organic layers were washed with brine (60 mL×3), dried over Na₂SO₄, filtered and concentrated under reduced pressure to give a residue. The residue was purified by column chromatography (SiO₂, DCM/EA=1/0 to 5/1) to the title compound (600 mg, 4% yield) as a black brown solid. LC-MS (ESI⁺) m/z 472.0 (M+H)⁺, ¹H NMR (400 MHz, DMSO-d₆) δ=10.85 (s, 1H), 7.54-7.49 (m, 2H), 7.33 (dd, J=3.2, 9.2 Hz, 1H), 6.64 (d, J=8.0 Hz, 1H), 6.58 (d, J=14.0 Hz, 1H), 5.13 (d, J=6.4 Hz, 1H), 4.27 (ddd, J=5.2, 6.8, 12.4 Hz, 1H), 3.80 (s, 3H), 3.40-3.34 (m, 4H), 3.07-2.98 (m, 4H), 2.80 (ddd. J=5.2, 13.2, 18.0, 1H), 2.56 (d, J=3.2 Hz, 1H), 2.17-2.09 (m, 1H), 1.98-1.87 (m, 1H).

### 3-((4-(4-(4-Bromo-2-fluoro-3-methylphenyl)piperazin-1-yl)-5-fluoro-2-methoxyphenyl)amino)piperidine-2,6-dione (Intermediate IE)

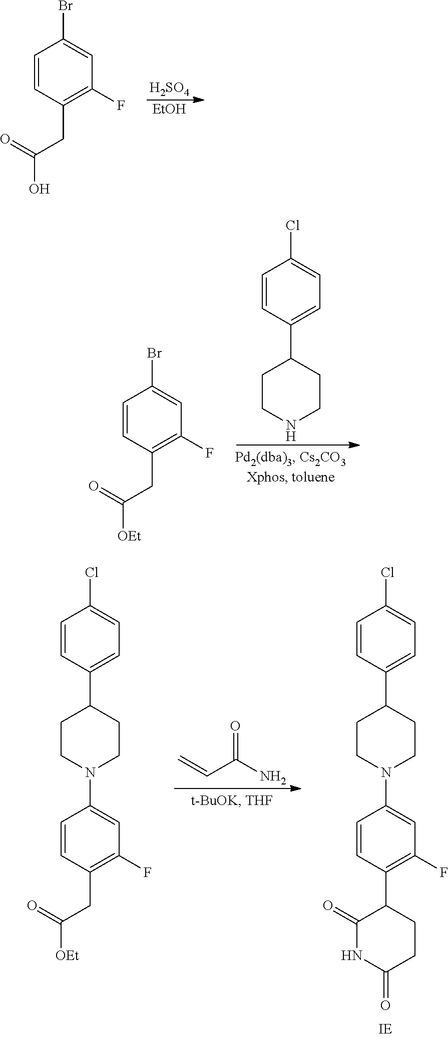

### Step 1—Ethyl 2-(4-bromo-2-fluorophenyl)acetate

To a solution of 2-(4-bromo-2-fluorophenyl)acetic acid (5.00 g, 21.4 mmol, CAS #114897-92-6) in EtOH (50 mL) was added H₂SO₄ (2.53 g, 25.7 mmol). The mixture was then stirred at 80° C. for 12 hrs. On completion, sat. Na₂CO₃ solution was added to the mixture until the pH=6, then the mixture was extracted with dichloromethane (3×60 mL). The combined organic layers were dried over anhydrous sodium sulfate, filtered and the filtrate was concentrated in vacuo to give the crude residue. The residue was purified by column chromatography (SiO₂, Petroleum ether/Ethyl acetate=20/1 to 10/1) to give the title compound (5.4 g, 80% yield) as a white solid. ¹H NMR (400 MHz, DMSO-d₆) δ=7.58 (dd, J=2.0, 9.6 Hz, 1H), 7.48-7.43 (m, 1H), 7.42-7.36 (m, 1H), 4.15 (q, J=7.2 Hz, 2H), 3.77 (s, 2H), 1.23 (t, J=7.2 Hz, 3H).

### Step 2—4-(4-(4-Bromo-2-fluoro-3-methylphenyl)piperazin-1-yl)-5-fluoro-2-methoxyaniline

A mixture of ethyl 2-(4-bromo-2-fluorophenyl)acetate (1.5 g, 5.8 mmol). 4-(4-chlorophenyl)piperidine (1.12 g, 5.75 mmol, CAS #26905-02-2), Pd₂(dba)₃ (263 mg, 287 μmol), XPhos (273 mg, 574 μmol) and Cs₂CO₃ (5.62 g, 17.2 mmol) in toluene (20 mL) was degassed and purged with N₂ three times. Then the mixture was stirred at 100° C. for 12 hrs under N₂ atmosphere. On completion, the reaction mixture was concentrated under reduced pressure to give a residue. The residue was purified by column chromatography (SiO₂, Petroleum ether/Ethyl acetate=20/1 to 10/1) to give the title compound (1 g, 44% yield) as an off-white solid. LC-MS (ESI⁺) m/z 376.8 (M+H)⁺.

### Step 3—3-((4-(4-(4-Bromo-2-fluoro-3-methylphenyl)piperazin-1-yl)-5-fluoro-2-methoxyphenyl)amino)piperidine-2,6-dione

To a solution of ethyl 2-(4-(4-(4-chlorophenyl)piperidin-1-yl)-2-fluorophenyl)acetate (800 mg, 2.13 mmol), acrylamide (302 mg, 4.26 mmol, CAS #9003-05-8) in THF (10 mL) was added tBuOK (262 mg, 2.34 mmol) at 0° C. The mixture was then stirred at 60° C. for 12 hrs. On completion, the reaction mixture was filtered and the filtrate was extracted with dichloromethane (3×20 mL). The combined organic layers were dried over anhydrous sodium sulfate, filtered and the filtrate was concentrated in vacuo to give the crude residue. The residue was purified by column chromatography (SiO₂, Petroleum ether/Ethyl acetate=3/1 to 1/1, DCM:EA=3:1) to give the title compound (200 mg, 21% yield) as an orange solid. LC-MS (ESI⁺) m/z 401.1 (M+H)⁺.

### 1-(5-(7-fluoro-4-(4,4,5,5-tetramethyl-1,3,2-dioxaborolan-2-yl)-1H-indazol-6-yl)-3,6-dihydropyridin-1 (2H)-yl)-3-(1H-1,2,3-triazol-1-yl)propan-1-one (Intermediate IF)

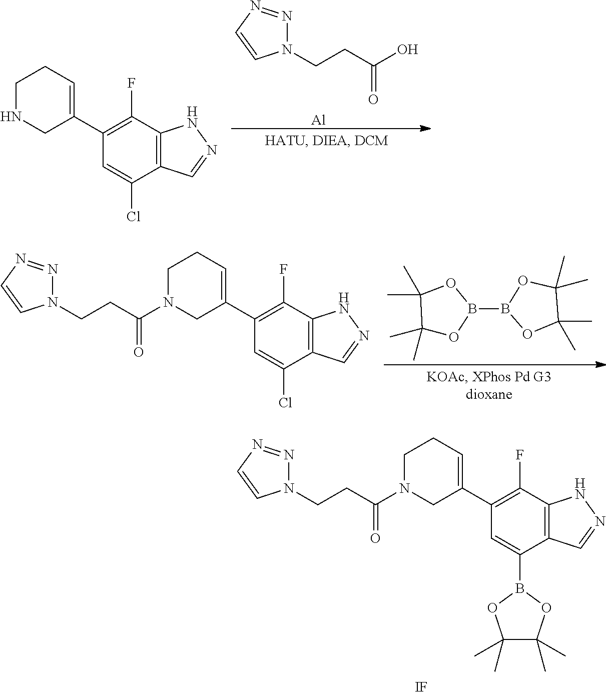

### Step 1—1-(5-(4-chloro-7-fluoro-1H-indazol-6-yl)-3,6-dihydropyridin-1 (2H)-yl)-3-(1H-1,2,3-triazol-1-yl)propan-1-one

To a solution of 3-(triazol-1-yl)propanoic acid (1.11 g, 7.87 mmol, Intermediate AI) in DMSO (20 mL) was added HATU (3.26 g, 8.58 mmol) and DIEA (3.70 g, 28.6 mmol, 4.98 mL). Next, 4-chloro-7-fluoro-6-(1,2,3,6-tetrahydropyridin-5-yl)-1H-indazole (1.8 g, 7.15 mmol, synthesized via Steps 1-2 of Intermediate GY) was added and the mixture was stirred at 25° C. for 1 hr. On completion, the reaction mixture was diluted with water (50 mL), then extracted with EA (50 mL×3), dried with anhydrous Na₂SO₄, filtered and the filtrate was concentrated under reduced pressure to obtain a residue. The crude product was purified by reversed-phase HPLC (0.1% FA condition) to give the title compound (1.8 g, 66% yield) as a white solid. LC-MS (ESI⁺) m/z 374.9 (M+H)⁺; ¹H NMR (400 MHz, CDCl3, 298 K) δ (ppm)=8.17-8.08 (s, 1H), 7.79-7.65 (m, 2H), 7.04-6.95 (s, 1H), 6.20-6.08 (d, J=17.6 Hz, 1H), 4.85-4.76 (m, 2H), 4.47-4.41 (s, 1H), 4.31-4.14 (s, 1H), 3.84-3.71 (s, 1H), 3.82-3.71 (m, 1H), 3.62-3.57 (d, J=5.2 Hz, 1H), 3.13-3.05 (m, 2H), 2.42-2.33 (s, 2H).

### Step 2—1-(5-(7-fluoro-4-(4,4,5,5-tetramethyl-1,3,2-dioxaborolan-2-yl)-1H-indazol-6-yl)-3,6-dihydropyridin-1 (2H)-yl)-3-(1H-1,2,3-triazol-1-yl)propan-1-one

To a solution of 1-[5-(4-chloro-7-fluoro-1H-indazol-6-yl)-3,6-dihydro-2H-pyridin-1-yl]-3-(triazol-1-yl)propan-1-one (0.5 g, 1.33 mmol) and 4,4,5,5-tetramethyl-2-(4,4,5,5-tetramethyl-1,3,2-dioxaborolan-2-yl)-1,3,2-dioxaborolane (1.02 g, 4.00 mmol, CAS #73183-34-3) in dioxane (5 mL) was added KOAc (393 mg, 4.00 mmol) and XPhos Pd G3 (113 mg, 133 μmol). The mixture was then stirred at 80° C. for 2 hr. On completion, the reaction mixture was diluted with water (30 mL) and extracted with EA (10 mL×3). The combined organic layers were washed with brine (10 mL), dried over Na₂SO₄, filtered and the filtrate was concentrated under reduced pressure to give a residue The residue was purified by column chromatography (SiO₂, Petroleum ether/Ethyl acetate=100:0 to 0:100) to give the title compound (130 mg, 21% yield) as a white solid. LC-MS (ESI⁺) m/z 466.9 (M+H)⁺.

### 4-((3S,4R)-4-(4-bromophenyl)-3-fluoropiperidin-1-yl)-5-fluoro-2-methoxyaniline (Intermediate IG) & 4-((3R,4S)-4-(4-bromophenyl)-3-fluoropiperidin-1-yl)-5-fluoro-2-methoxyaniline (Intermediate IH)

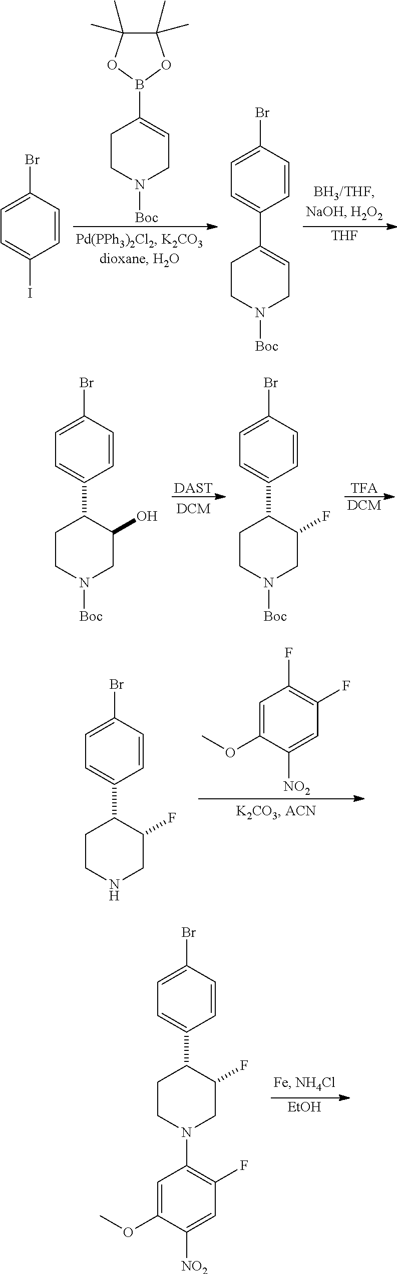

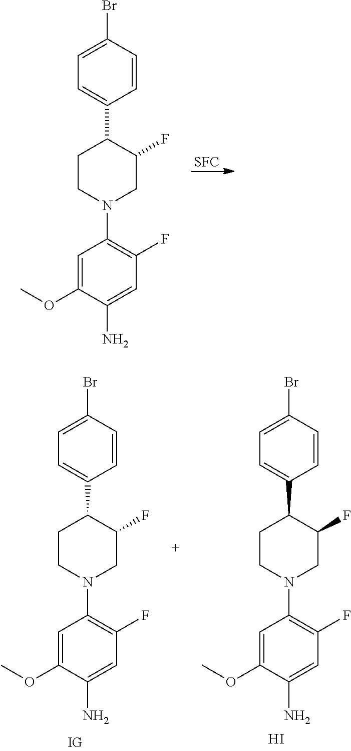

### Step 1—Tert-butyl 4-(4-bromophenyl)-5,6-dihydropyridine-1 (2H)-carboxylate

A solution of 1-bromo-4-iodobenzene (10.0 g, 35.3 mmol, CAS #589-87-7), tert-butyl 4-(4,4,5,5-tetramethyl-1,3,2-dioxaborolan-2-yl)-5,6-dihydropyridine-1 (2H)-carboxylate (10.9 g, 35.3 mmol, CAS #286961-14-6), CS₂CO₃ (34.5 g. 106 mmol), and Pd(PPh₃)₂Cl₂ (2.48 g. 3.53 mmol) in dioxane (200 mL) and H₂O (40 mL) was stirred at 80° C. for 4 hrs. On completion, the mixture was filtered and the organic filtrate was concentrated to give the crude residue. The residue was purified by column chromatography (SiO₂, Petroleum ether:Ethyl acetate=100/1 to 10/1) to give the title compound (9.80 g, 98% yield) as a brown oil. ¹H NMR (400 MHz. DMSO-d₆) δ=7.55-7.50 (m, 2H), 7.40-7.37 (m, 2H), 6.22-6.15 (m, 1H), 5.75 (s, 1H), 3.98 (d, J=2.0 Hz, 2H), 3.52 (t, J=5.6 Hz, 2H), 2.46-2.41 (m, 2H), 1.42 (s, 9H).

### Step 2—(3R,4R)-tert-butyl 4-(4-bromophenyl)-3-hydroxypiperidine-1-carboxylate

To a solution of tert-butyl 4-(4-bromophenyl)-5,6-dihydropyridine-1 (2H)-carboxylate (8.00 g, 23.6 mmol) in THF (10 mL) was added BH₃·THF (1 M, 35.4 mL) at 0° C. under N₂. The mixture was stirred at 25° C. for 12 hrs. Next, NaOH (2.37 g. 59.1 mmol) was added to the mixture at 0° C. After 1 hr of stirring, H₂O₂ (6.70 g, 59.1 mmol, 130 μL, 30% solution) was added dropwise to the mixture at 0° C. Then the mixture was stirred at 0° C. for 2 hrs. On completion, the reaction mixture was quenched by MeOH (10 mL), and stirred for 12 hrs at 25° C. Then the mixture was extracted with ethyl acetate (3×10 mL). The combined organic layers were washed with brine (15 mL) and dried over anhydrous sodium sulfate, filtered and the filtrate was concentrated in vacuo to give the crude residue. The residue was purified by column chromatography (SiO₂, Dichloromethane:Methanol=100/1) to give the title compound (6.70 g, 78% yield) as a yellow solid. ¹H NMR (400 MHz. DMSO-d₆) δ=7.52 (d, J=8.4 Hz, 2H), 7.27 (d, J=8.4 Hz, 2H), 4.92 (d, J=5.6 Hz, 1H), 4.24-4.11 (m, 1H), 4.01 (s, 1H), 3.55-3.43 (m, 1H), 2.87-2.72 (m, 1H), 2.54-2.49 (m, 1H), 1.79-1.69 (m, 1H), 1.65-1.54 (m, 1H), 1.48 (s, 9H).

### Step 3—(3S,4R)-tert-butyl 4-(4-bromophenyl)-3-fluoropiperidine-1-carboxylate

A solution of tert-butyl 4-(4-bromophenyl)-3-hydroxy-piperidine-1-carboxylate (4.70 g, 13.2 mmol) and DAST (6.38 g, 39.5 mmol, 5.23 mL) in DCM (50 mL) was stirred at 25° C. for 12 hrs. On completion, the reaction mixture was quenched with sat.NaHCO₃ (50 mL) and extracted with dichloromethane (3×50 mL). The combined organic layers were washed with brine (100 mL) and dried over anhydrous sodium sulfate, filtered and the filtrate was concentrated in vacuo to give the title compound (5.70 g) as a brown oil. LC-MS (ESI⁺) m/z 380.0 (M+Na)⁺.

### Step 4—(3S,4R)-4-(4-bromophenyl)-3-fluoropiperidine

A solution of tert-butyl 4-(4-bromophenyl)-3-fluoro-piperidine-1-carboxylate (5.50 g. 15.3 mmol) in DCM (25 mL) and TFA (10 mL) was stirred at 25° C. for 2 hrs. On completion, the reaction mixture was concentrated in vacuo to give the tittle compound (4.10 g) as a brown oil. LC-MS (ESI⁺) m/z 258.0 (M+H)⁺.

### Step 5—(3S,4R)-4-(4-bromophenyl)-3-fluoro-1-(2-fluoro-5-methoxy-4-nitrophenyl)piperidine

A solution of (3S,4R)-4-(4-bromophenyl)-3-fluoropiperidine (4.00 g. 15.5 mmol), 1,2-difluoro-4-methoxy-5-nitrobenzene (5.86 g, 31.0 mmol, CAS #100-02-7), and K₂CO₃ (10.7 g, 77.4 mmol) in DMF (40 mL) was stirred at 80° C. for 4 hrs. On completion, the reaction mixture was quenched with water (50 mL) and extracted with ethyl acetate (3×50 mL). The combined organic layers were washed with brine (60 mL) and dried over anhydrous sodium sulfate, filtered and the filtrate was concentrated in vacuo to give the crude residue. The residue was purified by column chromatography (SiO₂, Petroleum ether:Ethyl acetate=100/1 to 1/1) to give the title compound (4.00 g, 55% yield) as a yellow solid. LC-MS (ESI⁺)/z 428.7 (M+H)⁺.

### Step 6—4-((3S,4R)-4-(4-bromophenyl)-3-fluoropiperidin-1-yl)-5-fluoro-2-methoxyaniline

A solution of 4-(4-bromophenyl)-3-fluoro-1-(2-fluoro-5-methoxy-4-nitro-phenyl)piperidine (3.60 g. 8.43 mmol), NH4Cl (4.51 g, 84.26 mmol), and Fe (4.71 g, 84.2 mmol) in EtOH (50 mL), H₂O (15 mL) was stirred at 80° C. for 2 hrs. On completion, the reaction mixture was filtered and the filtrate was concentrated in vacuo to give the crude residue. The crude residue was purified by reversed-phase HPLC (0.8 g/L ammonium bicarbonate) to give the tittle compound (1.20 g, 40% yield) as a brown solid. LC-MS (ESI⁺)/z 398.6 (M+H)⁺.

### Step 7—4-((3S,4R)-4-(4-bromophenyl)-3-fluoropiperidin-1-yl)-5-fluoro-2-methoxyaniline & 4-((3R4S)-4-(4-bromophenyl)-3-fluoropiperidin-1-yl)-5-fluoro-2-methoxyaniline

4-((3S,4R)-4-(4-bromophenyl)-3-fluoropiperidin-1-yl)-5-fluoro-2-methoxyaniline was separated by SFC (Column: Chiralpak AD-3 50×4.6 mm I.D., 3 um, Mobile phase: Phase A for CO₂, and Phase B for MeOH (0.05% DEA); Gradient elution: B in A from 5% to 40%, Flow rate: 3 mL/min; Detector: PDA; Column Temp: 35° C.: Back Pressure: 100 Bar) to give 4-((3S,4R)-4-(4-bromophenyl)-3-fluoropiperidin-1-yl)-5-fluoro-2-methoxyaniline (400 mg) as a brown solid and 4-((3R,4S)-4-(4-bromophenyl)-3-fluoropiperidin-1-yl)-5-fluoro-2-methoxyaniline (400 mg) as a brown solid. The absolute stereochemistry of the diastereomers was assigned arbitrarily. LC-MS (ESI⁺)/z 398.6 (M+H)⁺ for both isomers.

### 3-((4-((3S,4R)-4-(4-bromophenyl)-3-fluoropiperidin-1-yl)-5-fluoro-2-methoxyphenyl)amino)piperidine-2,6-dione (Intermediate II)

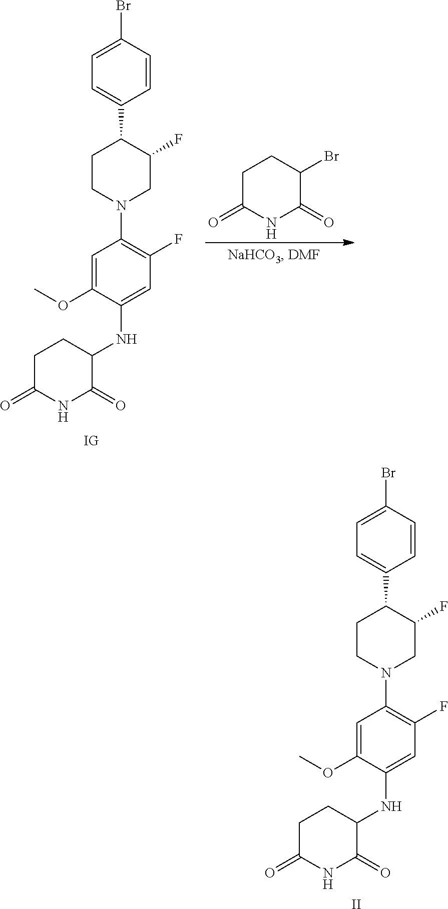

A solution of 4-((3S,4R)-4-(4-bromophenyl)-3-fluoropiperidin-1-yl)-5-fluoro-2-methoxyaniline (200 mg, 503 μmol, Intermediate IG), 3-bromopiperidine-2,6-dione (193 mg, 1.01 mmol, CAS #62595-74-8), and NaHCO₃ (211 mg, 2.52 mmol) in DMF (2 mL) was stirred at 80° C. for 12 hrs. On completion, the reaction mixture was quenched with water (20 mL) and extracted with ethyl acetate (3×20 mL). The combined organic layers were washed with brine (20 mL) and dried over anhydrous sodium sulfate, filtered and the filtrate was concentrated in vacuo to give the crude residue. The residue was purified by column chromatography (SiO₂. Petroleum ether:Ethyl acetate=100/1 to 1/1) to give the tittle compound (60.0 mg) as a green oil. LC-MS (ESI⁺)/z 509.8 (M+H)⁺.

### 4-((1s,4s)-4-(4-Chlorophenyl)cyclohexyl)-5-fluoro-2-methoxyaniline (Intermediate IJ) & 4-((1r,4r)-4-(4-chlorophenyl)cyclohexyl)-5-fluoro-2-methoxyaniline (Intermediate IK)

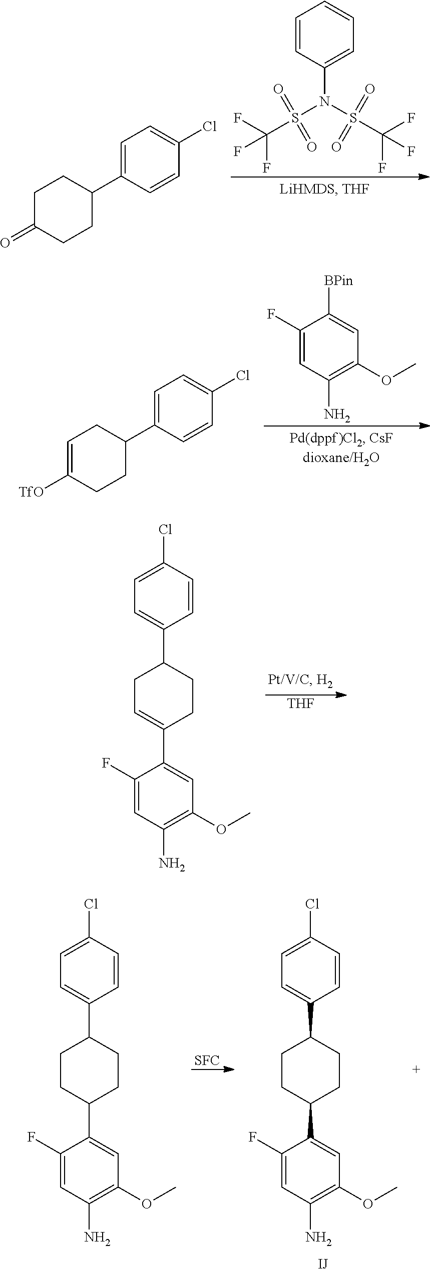

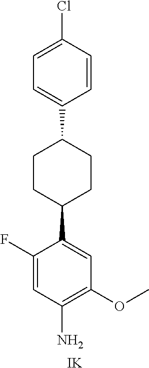

### Step 1—4′-Chloro-1,2,3,6-tetrahydro-[1,1′-biphenyl]-4-yl trifluoromethanesulfonate

To a solution of 4-(4-chlorophenyl)cyclohexan-1-one (15 g, 71.8 mmol, CAS #14472-80-1) in THF (150 mL) was added LiHMDS (1 M, 79.0 mL) at −78° C. under nitrogen atmosphere and the mixture was stirred for 1 hour under nitrogen atmosphere. Then 1,1,1-trifluoro-N-phenyl-N-((trifluoromethyl)sulfonyl)methanesulfonamide (33.3 g, 93.4 mmol, CAS #37595-74-7) in THF (150 mL) was added at −78° C., and the mixture was stirred for 1 hr at 0° C. under nitrogen atmosphere. On completion, the reaction mixture was quenched by NH4CL (80 mL) and extracted with ethyl acetate (3×80 mL). The combined organic layers were washed with brine (100 mL) and dried over anhydrous sodium sulfate, filtered and the filtrate was concentrated in vacuo to give the crude residue. The residue was purified by column chromatography (SiO₂, Petroleum ether/Ethyl acetate=1/0) to give the title compound (21 g, 73% yield) as colorless oil. ¹H NMR (400 MHz, CDCl₃-d) 6=7.30-7.26 (m, 2H), 7.14 (d, J=8.4 Hz, 2H), 5.85-5.82 (m, 1H), 2.87-2.80 (m, 1H), 2.60-2.20 (m, 5H), 1.97-1.85 (in, 1H).

### Step 2—4″-Chloro-2-fluoro-5-methoxy-2′,3′,4′,5′-tetrahydro-[1,1′:4′,1″-terphenyl]-4-amine

To a solution of 4′-chloro-1,2,3,6-tetrahydro-[1,1′-biphenyl]-4-yl trifluoromethanesulfonate (5.8 g, 17 mmol) and 5-fluoro-2-methoxy-4-(4,4,5,5-tetramethyl-1,3-dioxolan-2-yl)aniline (4.5 g, 17 mmol, CAS #1326283-60-6) in dioxane (60 mL) and H₂O (12 mL) was added Pd(dppf)Cl₂ (1.25 g, 1.70 mmol) and CsF (7.76 g, 51.0 mmol) at 25° C. under nitrogen atmosphere, then the mixture was stirred at 80° C. for 2 hrs under nitrogen atmosphere. On completion, the reaction mixture was quenched with water (50 mL) and extracted with ethyl acetate (3×40 mL). The combined organic layers were washed with brine (50 mL) and dried over anhydrous sodium sulfate, filtered and the filtrate was concentrated in vacuo to give the crude residue. The residue was purified by column chromatography (SiO₂, Petroleum ether/Ethyl acetate=10/1 to 3/1) to give the title compound (4.3 g, 76% yield) as yellow oil. LC-MS (ESI⁺) m/z 332.1 (M+H)⁺.

### Step 3—4-(4-(4-Chlorophenyl)cyclohexyl)-5-fluoro-2-methoxyaniline

To a solution of 4″-chloro-2-fluoro-5-methoxy-2′,3′,4′,5′-tetrahydro-[1,1′:4′,1″-terphenyl]-4-amine (3 g, 9.04 mmol) in anhydrous THF (30 mL) was added Pt/V/C (3 g, 574 μmol, 5 wt %) at 25° C. was degassed and purged with H₂ three times, and then the reaction was stirred at 25° C. under H₂ atmosphere for 12 h. On completion, the reaction was filtered through kieselguhr carefully, and the filtrate was concentrated in vacuo to give a residue. The crude residue was purified by reversed-phase HPLC (0.1% FA condition) to give the title compound (1.3 g, 37% yield, FA) as a colorless oil. LC-MS (ESI⁺) m/z 334.1 (M+H)⁺.

### Step 4—4-((1s,4s)-4-(4-Chlorophenyl)cyclohexyl)-5-fluoro-2-methoxyaniline & 4-((1r,4r)-4-(4-chlorophenyl)cyclohexyl)-5-fluoro-2-methoxyaniline

4-(4-(4-Chlorophenyl)cyclohexyl)-5-fluoro-2-methoxyaniline (580 mg, 785 μmol) was separated by SFC (column: DAICEL CHIRALPAK IK (250 mm×30 mm, 10 um):mobile phase: [CO₂-MeOH (0.1% NH₃H₂O)]; B %: 45%, isocratic elution mode) to give the first eluting isomer 4-((1s,4s)-4-(4-Chlorophenyl)cyclohexyl)-5-fluoro-2-methoxyaniline (900 mg, 58% yield as a colorless oil and the second eluting isomer 4-((1r,4r)-4-(4-chlorophenyl)cyclohexyl)-5-fluoro-2-methoxyaniline (370 mg, 24% yield) as a colorless oil. Absolute stereochemistry of the diastereomers was assigned arbitrarily. LC-MS (ESI⁺) m/z 334.1 (M+H)⁺ for both isomers.

### 3-((4-((1s,4s)-4-(4-Chlorophenyl)cyclohexyl)-5-fluoro-2-methoxyphenyl)amino)piperidine-2,6-dione (Intermediate IL)

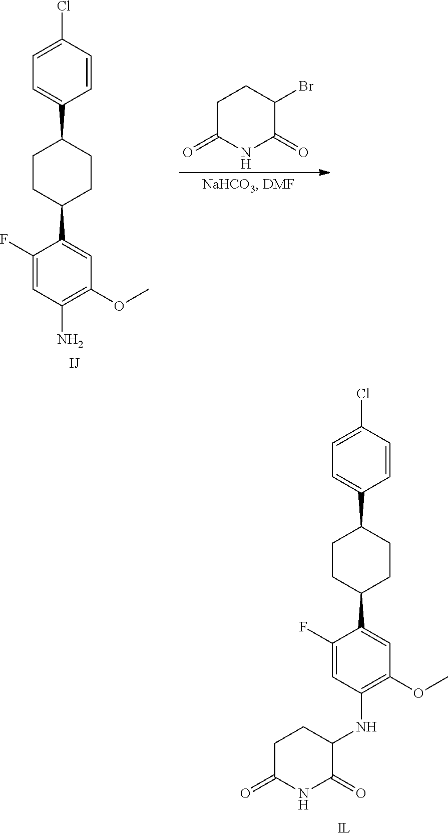

To a solution of 4-((1s,4s)-4-(4-chlorophenyl)cyclohexyl)-5-fluoro-2-methoxyaniline (800 mg, 2.40 mmol, Intermediate IJ) and 3-bromopiperidine-2,6-dione (920 mg, 4.79 mmol, CAS #62595-74-8) in anhydrous DMF (8 mL) was added NaHCO₃ (805 mg, 9.59 mmol, 372 L) at 25° C., then the reaction was stirred at 80° C. for 12 hrs. On completion, the reaction mixture was quenched with water (20 mL) and extracted with ethyl acetate (3×15 mL). The combined organic layers were washed with brine (20 mL) and dried over anhydrous sodium sulfate, filtered and the filtrate was concentrated in vacuo to give the crude residue. The residue was purified by column chromatography (SiO₂, Petroleum ether/Ethyl acetate=5/1 to 3/1) to give the title compound (800 mg, 75% yield) as a colorless oil. LC-MS (ESI⁺) m/z 445.2 (M+H)⁺.

### 3-((4-((1r,4r)-4-(4-chlorophenyl)cyclohexyl)-5-fluoro-2-methoxyphenyl)amino)piperidine-2,6-dione (Intermediate IM)

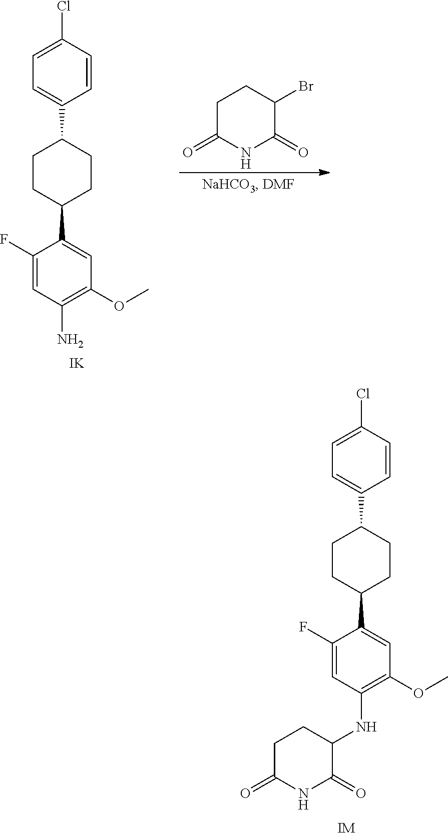

To a solution of 4-((1r,4r)-4-(4-chlorophenyl)cyclohexyl)-5-fluoro-2-methoxyaniline (207 mg, 1.08 mmol, Intermediate IK) and 3-bromopiperidine-2,6-dione (360 mg, 1.08 mmol, CAS #62595-74-8) in anhydrous DMF (8 mL) was added NaHCO₃ (362 mg, 4.31 mmol, 167 L) at 25° C., then the reaction was stirred at 80° C. for 12 hrs. On completion, the reaction mixture was quenched with water (20 mL) and extracted with ethyl acetate (3×15 mL). The combined organic layers were washed with brine (20 mL) and dried over anhydrous sodium sulfate, filtered and the filtrate was concentrated in vacuo to give the crude residue. The residue was purified by column chromatography (SiO₂. Petroleum ether/Ethyl acetate=5/1 to 3/1) to give the title compound (370 mg, 53% yield) as a colorless oil. LC-MS (ESI⁺) m/z 445.1 (M+H)⁺.

### (4S)-4-(4-chloro-3-methyl-phenyl)-3,3-difluoro-1-(2-fluoro-5-methoxy-4-nitro-phenyl)piperidine (Intermediate IN) & (4R)-4-(4-chloro-3-methyl-phenyl)-3,3-difluoro-1-(2-fluoro-5-methoxy-4-nitro-phenyl)piperidine (Intermediate 10)

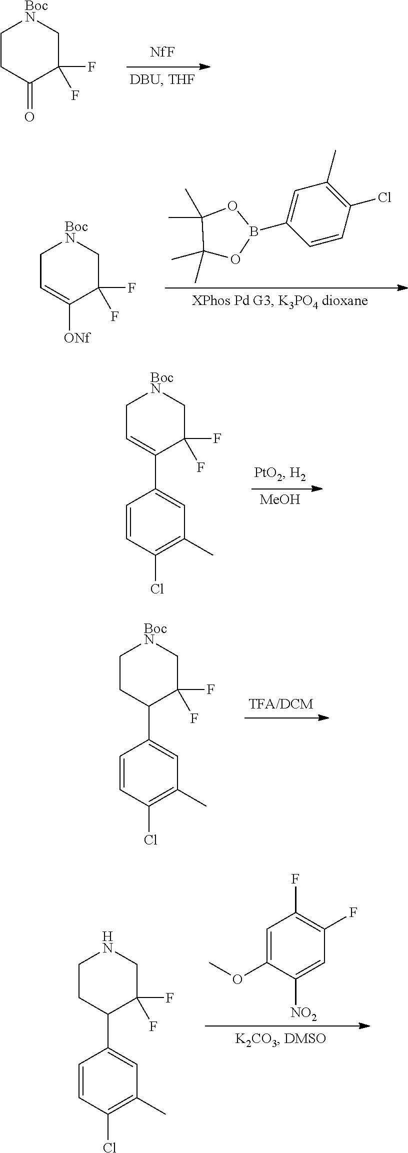

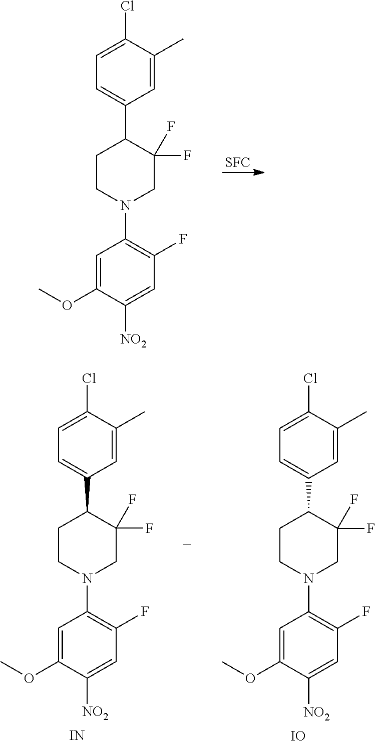

### Step 1—tert-butyl (4-bromo-3-methoxyphenyl)carbamate

To a solution of 4-bromo-3-methoxyaniline (3 g, 15 mmol, CAS #19056-40-7) in MeOH (40 mL) was added (Boc)₂O (3.89 g, 17.8 mmol, 4.09 mL). The mixture was then stirred at 70° C. for 12 hrs. On completion, the reaction mixture was concentrated in vacuo and diluted with water (20 mL) and extracted with dichloromethane (2×20 mL). The combined organic layers were washed with brine (40 mL) and dried over anhydrous sodium sulfate, filtered and the filtrate was concentrated in vacuo to give the title compound (4.5 g) as a brown solid. LC-MS (ESI⁺) m/z 245.8 (M−55)⁺.

### Step 2—Tert-butyl (4-bromo-3-methoxyphenyl)(methyl)carbamate

To a solution of tert-butyl (4-bromo-3-methoxyphenyl)carbamate (300 mg, 992 μmol) in THF (20 mL) was added NaH (47.6 mg, 1.19 mmol, 60% dispersion in mineral oil) and CH₃I (169 mg, 1.19 mmol, 297 μL) dropwise at 0° C., and the mixture was stirred for 0.5 hrs. Then the mixture was stirred at 25° C. for 1 hr. On completion, the reaction mixture was quenched with water (20 mL) and extracted with dichloromethane (2×20 mL). The combined organic layers were washed with brine (20 mL) and dried over anhydrous sodium sulfate, filtered and the filtrate was concentrated in vacuo to give the title compound (300 mg) as a brown oil. LC-MS (ESI⁺) m/z 260.0 (M−55)⁺.

### Step 3—Tert-butyl 4-(4-chloro-3-methylphenyl)-3,3-difluoropiperidine-1-carboxylate

A mixture of tert-butyl 4-(4-chloro-3-methylphenyl)-3,3-difluoro-3,6-dihydropyridine-1 (2H)-carboxylate (2 g, 6 mmol) in MeOH (30 mL) was added PtO₂ (1.32 g, 5.82 mmol). The mixture was then stirred at 25° C. for 12 hrs under H₂ (20 psi). On completion, the reaction mixture was filtered and concentrated under reduced pressure to give the title compound (2 g) as a yellow solid. LC-MS (ESI⁺) m/z 290.1 (M−55)⁺.

### Step 4—4-(4-chloro-3-methylphenyl)-3.3 difluoropiperidine

A mixture of tert-butyl 4-(4-chloro-3-methylphenyl)-3,3-difluoropiperidine-1-carboxylate (2 g, 6 mmol) in DCM (20 mL) was added TFA (9.21 g, 80.7 mmol) and the mixture was stirred at 25° C. for 2 hrs. On completion, the reaction mixture was concentrated under reduced pressure to remove DCM to give the title compound (700 mg) as a yellow oil. LC-MS (ESI⁺) m/z 246.0 (M+H)⁻.

### Step 5—4-(4-chloro-3-methylphenyl)-3.3 difluoro-1-(2-fluoro-5-methoxy-4-nitrophenyl)piperidine

To a solution of 4-(4-chloro-3-methylphenyl)-3.3 difluoropiperidine (700 mg, 2.85 mmol) in DMSO (7 mL) was added K₂CO₃ (1.18 g, 8.55 mmol) and 1,2-difluoro-4-methoxy-5-nitrobenzene (646.6 mg, 3.42 mmol). The mixture was then stirred at 80° C. for 2 hrs. On completion, the reaction mixture was filtered and the filtrate was concentrated in vacuo to give the crude residue. The crude residue was purified by reversed-phase HPLC (0.1% FA condition) to give the title compound (400 mg, 34% yield) as an yellow solid. LC-MS (ESI⁺) m/z 415.0 (M+H)⁺.

### Step 6—(S)-4-(4-chloro-3-methylphenyl)-3,3-difluoro-1-(2-fluoro-5-methoxy-4-nitrophenyl)piperidine & (R)-4-(4-chloro-3-methylphenyl)-3,3-difluoro-1-(2-fluoro-5-methoxy-4-nitrophenyl)piperidine

A solution of 4-(4-chloro-3-methylphenyl)-3.3 difluoro-1-(2-fluoro-5-methoxy-4-nitrophenyl)piperidine (400 mg, 964.31 μmol) was separated by SFC (column: DAICEL CHIRALPAK AS (250 mm*30 mm, 10 um); mobile phase: [CO2-MeOH (0.1% NH₃H2O)]; B %:35%, isocratic elution mode) to give the first eluting isomer (S)-4-(4-chloro-3-methylphenyl)-3,3-difluoro-1-(2-fluoro-5-methoxy-4-nitrophenyl)piperidine (170 mg, 43% yield) as an yellow solid and the second eluting isomer (R)-4-(4-chloro-3-methylphenyl)-3,3-difluoro-1-(2-fluoro-5-methoxy-4-nitrophenyl)piperidine (170 mg, 43% yield) as an yellow solid. LC-MS (ESI⁺) m/z 415.0 (M+H)⁻ for both isomers. The absolute stereochemistry of the enantiomers were assigned arbitrarily.

### (S)-4-(4-(4-chloro-3-methylphenyl)-3,3-difluoropiperidin-1-yl)-5-fluoro-2-methoxyaniline (Intermediate IP)

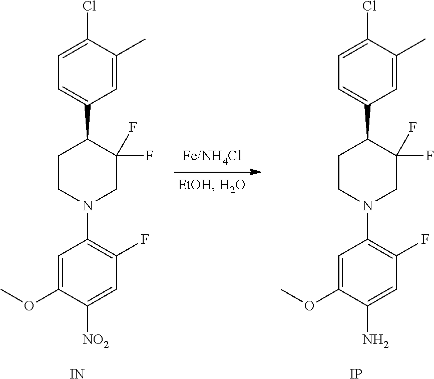 [SMILES: COc1cc(N2CC[C@@H](c3ccc(Cl)c(C)c3)C(F)(F)C2)c(F)cc1[N+](=O)[O-] ; COc1cc(N2CC[C@@H](c3ccc(Cl)c(C)c3)C(F)(F)C2)c(F)cc1N ; "IN" ; "IP"]

To a solution of (4S)-4-(4-chloro-3-methyl-phenyl)-3,3-difluoro-1-(2-fluoro-5-methoxy-4-nitro-phenyl)piperidine (150 mg, 362 μmol, Intermediate IN) in EtOH (2 mL) H₂O (2 mL) was added Fe (101 mg, 1.80 mmol) and NH₄Cl (155 mg. 2.80 mmol). The mixture was then stirred at 80° C. for 2 hrs. On completion, the residue was diluted with water (10 mL) and extracted with EA (10 mL×2). The combined organic layers were washed with brine (10 mL×2), dried over Na₂SO₄, filtered and concentrated under reduced pressure to give the title compound (135 mg) as a white solid. LC-MS (ESI⁺) m/z 385.1 (M+H)⁺.

### 3-((4-((S)-4-(4-chloro-3-methylphenyl)-3,3-difluoropiperidin-1-yl)-5-fluoro-2-methoxyphenyl)amino)piperidine-2,6-dione (Intermediate IQ)

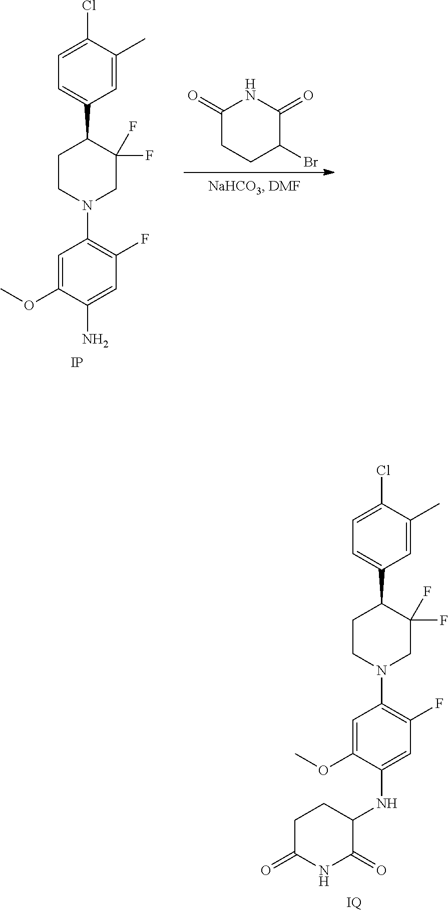

To a solution of 4-[(4S)-4-(4-chloro-3-methyl-phenyl)-3,3-difluoro-1-piperidyl]-5-fluoro-2-methoxy-aniline (115 mg, 299 μmol, Intermediate IP) in DMF (0.5 mL) was added 3-bromopiperidine-2,6-dione (173 mg, 897 μmol, CAS #62595-74-8), NaHCO₃ (50.2 mg, 598 μmol). The mixture was then stirred at 80° C. for 24 hrs. On completion, the reaction mixture was diluted with water (5 mL) and extracted with EA (5 mL×2). The combined organic layers were washed with brine (5 mL×2), dried over Na₂SO₄, filtered and concentrated under reduced pressure to give a residue to give the title compound (150 mg) as a white solid. LC-MS (ESI⁺) m/z 496.2 (M+H)⁺.

### 4-(4-(4-Chloro-3-fluorophenyl)cyclohexyl)-5-fluoro-2-methoxyaniline (Intermediate IR)

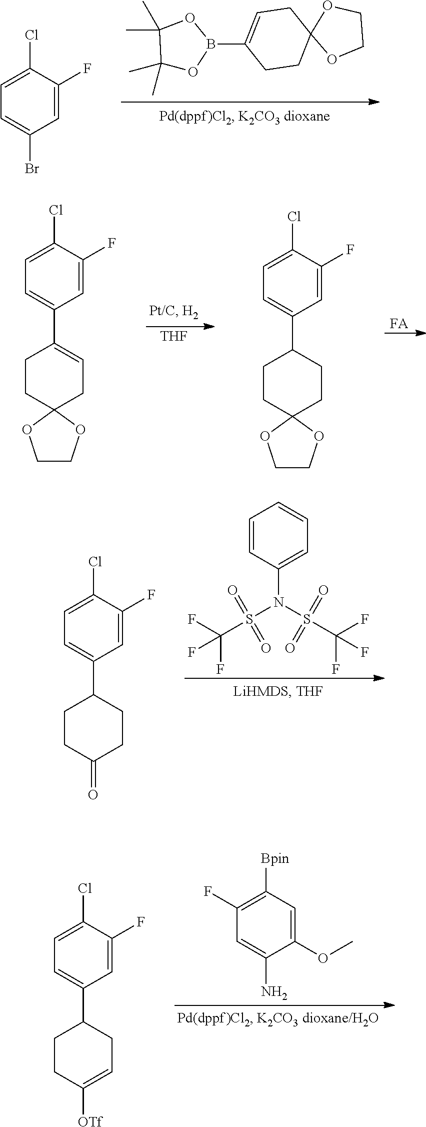

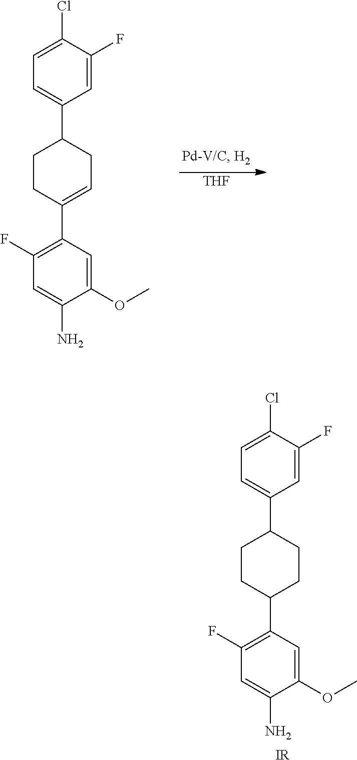

### Step 1—8-(4-Chloro-3-fluorophenyl)-1,4-dioxaspiro[4.5]dec-7-ene

To a solution of 4-bromo-1-chloro-2-fluoro-benzene (10 g, 50 mmol, CAS #60811-18-9) and 2-(1,4-dioxaspiro[4.5]dec-7-en-8-yl)-4,4,5,5 tetramethyl-1,3,2-dioxaborolane (12.7 g, 47.8 mmol, CAS #680596-79-6) in dioxane (100 mL) and H₂O (20 mL) was added Pd(dppf)Cl₂·CH₂Cl₂ (3.90 g, 4.78 mmol) and K₂CO₃ (6.60 g, 47.8 mmol). The mixture was then stirred at 80° C. for 12 hrs. On completion, the reaction mixture was concentrated under reduced pressure to remove solvent to give the crude residue. The residue was purified by column chromatography (SiO₂, Petroleum ether:Ethyl acetate=10/1) to give the title compound (11.5 g, 85% yield) as an orange oil. LC-MS (ESI⁺) m/z 269.1 (M+H)⁺.

### Step 2—8-(4-Chloro-3-fluorophenyl)-1.4 dioxaspiro[4,5]decane

To a solution of 8-(4-chloro-3-fluoro-phenyl)-1,4-dioxaspiro[4.5]dec-7-ene (9.00 g, 33.5 mmol) in THF (40 mL) was added Pt/C under N₂ atmosphere. The suspension was degassed and purged with H₂ three times. The mixture was then stirred under H₂ (40 psi) at 50° C. for 12 hrs. On completion, the reaction mixture was concentrated under reduced pressure to remove solvent to give the title compound (9 g) as a white solid. LC-MS (ESI⁺) m/z 270.8 (M+H)⁺.

### Step 3—4-(4-Chloro-3-fluorophenyl)cyclohexan-1-one

A solution of 8-(4-chloro-3-fluoro-phenyl)-1,4-dioxaspiro[4.5]decane (8 g, 30 mmol) in HCOOH (80 mL) was stirred at 25° C. for 12 hrs. On completion, the reaction mixture was concentrated under reduced pressure to remove solvent, then the mixture was extracted by dichloromethane (3×100 mL). The combined organic layers were washed by brine (200 mL) and dried over anhydrous sodium sulfate, filtered and the filtrate was concentrated in vacuo to give the title compound (9 g) as a yellow oil. LC-MS (ESI⁺) m/z 227.1 (M+H)⁺

### Step 4—4′-Chloro-3′-fluoro-1,2,3,6-tetrahydro-[1,1′-biphenyl]-4-yl trifluoromethanesulfonate

To a solution of 4-(4-chloro-3-fluoro-phenyl)cyclohexanone (8 g, 40 mmol) in THF (50 mL) was added LiHMDS (1 M, 35.3 mL) at 0° C. under N₂ atmosphere and the mixture was stirred for 1 hr. Next, 1,1,1-trifluoro-N-phenyl-N-(trifluoromethylsulfonyl)methanesulfonamide (16.4 g. 45.9 mmol. CAS #37595-74-7) was added and the mixture was stirred at 0° C. for 3 hrs. On completion, the reaction mixture was concentrated under reduced pressure to remove solvent, then the mixture was extracted by ethyl acetate (3×100 mL). The combined organic layers were washed by brine (200 mL) and dried over anhydrous sodium sulfate, filtered and the filtrate was concentrated in vacuo. Then the mixture was purified by column chromatography (SiO₂, Petroleum ether:Ethyl acetate=10/1) to give the title compound (3.2 g, 23% yield) as a white solid. ¹H NMR (400 MHz, DMSO-d₆) δ=7.49 (t, J=8.0 Hz, 1H), 7.37 (dd, J=2.0, 10.8 Hz, 1H), 7.20-7.13 (m, 1H), 6.01-5.92 (m, 1H), 2.92-2.81 (m, 1H), 2.37-2.25 (m, 2H), 1.98-1.87 (m, 2H), 0.87-0.79 (m, 2H).

### Step 5—4″-Chloro-2,3″-difluoro-5-methoxy-2′,3′,4′,5′-tetrahydro-[1,1′:4′,1″-terphenyl]-4-amine

To a solution of [4-(4-chloro-3-fluoro-phenyl)cyclohexen-1-yl]trifluoromethanesulfonate (2.00 g, 5.58 mmol) and 5-fluoro-2-methoxy-4-(4,4,5,5-tetramethyl-1,3,2-dioxaborolan-2-yl)aniline (1.86 g, 5.58 mmol, CAS #1326283-60-6) in dioxane (30 mL) and H₂O (6 mL) was added Pd(dppf)Cl (612 mg, 836 μmol) and K₂CO₃ (2.31 g, 16.7 mmol). The mixture was then stirred at 80° C. for 2 hrs. On completion, the reaction mixture was concentrated under reduced pressure to remove solvent, then the mixture was extracted with ethyl acetate (3×20 mL). The combined organic layers were washed with brine (40 mL) and dried over anhydrous sodium sulfate, filtered and the filtrate was concentrated in vacuo. Then the mixture was purified by column chromatography (SiO₂, Petroleum ether:Ethyl acetate=4/1) to give the title compound (1.8 g, 85% yield) as a yellow oil. LC-MS (ESI⁺) m/z 350.0 (M+H)⁺.

### Step 6—4-(4-(4-Chloro-3-fluorophenyl)cyclohexyl)-5-fluoro-2-methoxyaniline

To a solution of 4-[4-(4-chloro-3-fluoro-phenyl)cyclohexen-1-yl]-5-fluoro-2-methoxy-aniline (1.60 g, 4.57 mmol) in THF (25 mL) was added Pt-V/C (1.6 g, 10 wt %) under N₂ atmosphere. The suspension was degassed and purged with H₂ 3 times. Then the mixture was stirred under H₂ (15 psi) at 25° C. for 12 hrs. On completion, the reaction mixture was purified by reversed-phase HPLC (0.8 g/L ammonium bicarbonate) to give the title compound (1 g, 62% yield) as a yellow oil. LC-MS (ESI⁺) m/z 352.1 (M+H)⁺.

### 4-((1S,4S)-4-(4-Chloro-3-fluorophenyl)cyclohexyl)-5-fluoro-2-methoxyaniline (Intermediate IS) and 4-((1R,4R)-4-(4-chloro-3-fluorophenyl)cyclohexyl)-5-fluoro-2-methoxyaniline (Intermediate IT)

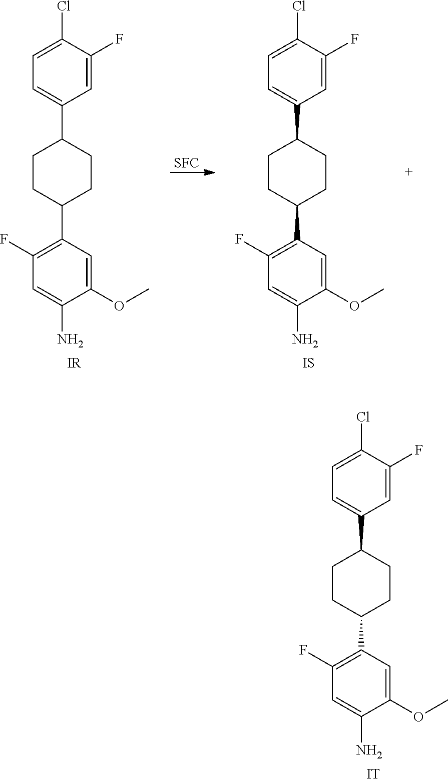

4-(4-(4-Chloro-3-fluorophenyl)cyclohexyl)-5-fluoro-2-methoxyaniline (1 g. 2.84 mmol, Intermediate IR) was separated by SFC (column: DAICEL CHIRALPAK IC (250 mm*30 mm, 10 um); mobile phase: [CO₂-MeOH]; B %:50%, isocratic elution mode) to give the first eluting isomer 4-((1S,4S)-4-(4-chloro-3-fluorophenyl)cyclohexyl)-5-fluoro-2-methoxyaniline (520 mg, 52% yield) and the second eluting isomer 4-((1R,4R)-4-(4-chloro-3-fluorophenyl)cyclohexyl)-5-fluoro-2-methoxyaniline (160 mg, 16% yield) as orange oils. LC-MS (ESI⁺) m/z 352.1 (M+H)⁺ for both isomers. The absolute stereochemistry of the diastereomers was assigned arbitrarily.

### 3-((4-((1s,4s)-4-(4-Chloro-3-fluorophenyl)cyclohexyl)-5-fluoro-2-methoxyphenyl)amino)piperidine-2,6-dione (Intermediate IU)

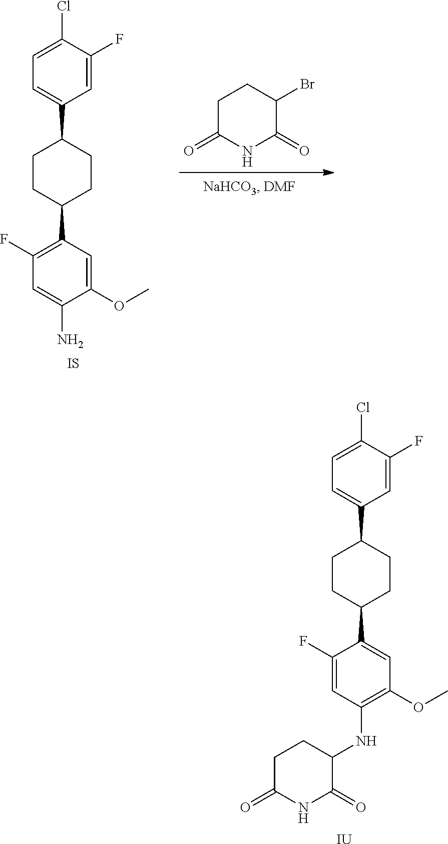

To a solution of 4-((1S,4S)-4-(4-chloro-3-fluorophenyl)cyclohexyl)-5-fluoro-2-methoxyaniline (200 mg, 568 μmol, Intermediate IS) and 3-bromopiperidine-2,6-dione (218 mg, 1.14 mmol. CAS #62595-74-8) in DMF (4 mL) was added NaHCO₃ (191 mg, 2.27 mmol). The mixture was then stirred at 80° C. for 12 hrs. On completion, the reaction mixture was concentrated under reduced pressure to remove solvent, then the mixture was extracted by ethyl acetate (3×20 mL). The combined organic layers were washed with brine (40 mL) and dried over anhydrous sodium sulfate, filtered and the filtrate was concentrated in vacuo. Then the mixture was purified by column chromatography (SiO₂, Petroleum ether:Ethyl acetate=4/1) to give the title compound (130 mg, 49% yield) as a yellow solid. LC-MS (ESI⁺) m/z 463.1 (M+H)⁺.

### 3-((4-((1r,4r)-4-(4-Chloro-3-fluorophenyl)cyclohexyl)-5-fluoro-2-methoxyphenyl)amino)piperidine-2,6-dione (Intermediate IV)

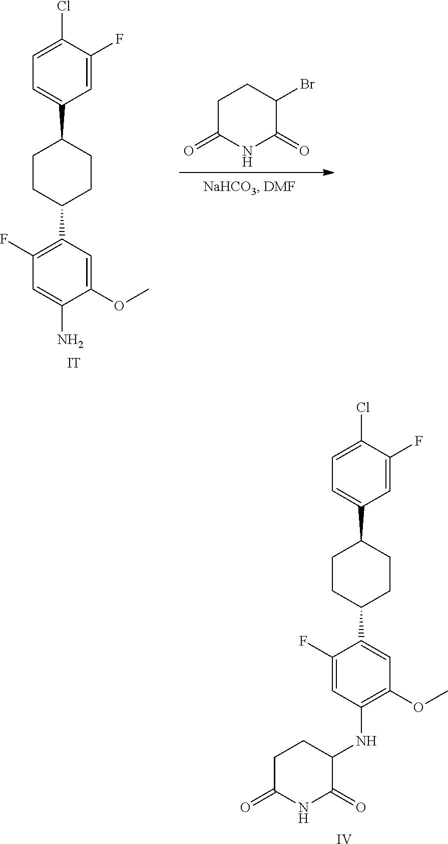

To a solution of 4-((1R,4R)-4-(4-chloro-3-fluorophenyl)cyclohexyl)-5-fluoro-2-methoxyaniline (160 mg, 455 μmol, Intermediate IT) and 3-bromopiperidine-2,6-dione (175 mg, 910 μmol, CAS #62595-74-8) in DMF (4 mL) was added NaHCO₃ (153 mg, 1.82 mmol). The mixture was stirred at 80° C. for 12 hrs. On completion, the reaction mixture was quenched by addition of sat. NH4Cl (5 mL), and then extracted with EtOAc (10 mL×3). The combined organic layers were washed with brine (10 mL×3), dried over Na₂SO₄, filtered and concentrated under reduced pressure to give a residue. The crude residue was purified by reversed-phase HPLC (0.1% FA condition) to give the title compound (90 mg, 39% yield) as a white solid. LC-MS (ESI⁺) m/z 463.1 (M+H)⁻.

### 4-(1-(4-Chloro-2-fluoro-3-methylphenyl)piperidin-4-yl)-5-fluoro-2-methoxyaniline (Intermediate IW)

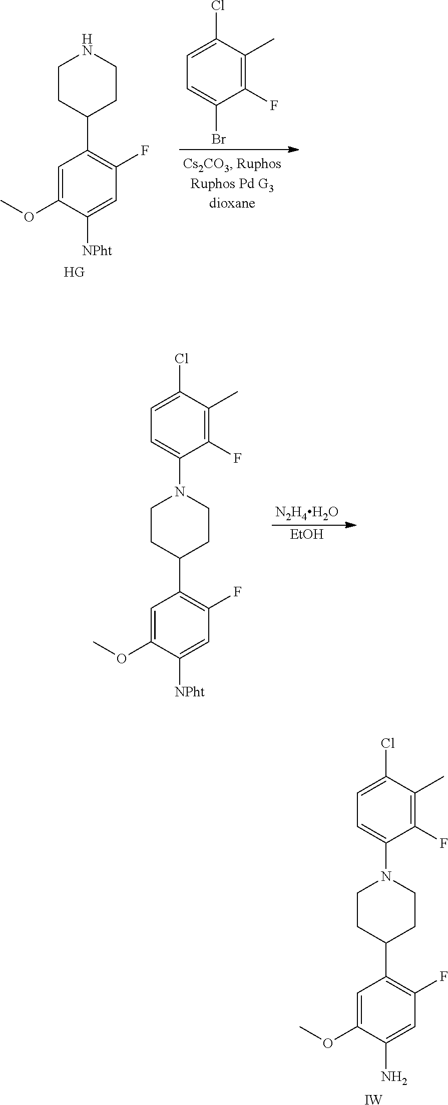

### Step 1—2-(4-(1-(4-Chloro-2-fluoro-3-methylphenyl)piperidin-4-yl)-5-fluoro-2-methoxyphenyl)isoindoline-1,3-dione

A mixture of 2-[5-fluoro-2-methoxy-4-(4-piperidyl)phenyl]isoindoline-1,3-dione (700 mg, 1.98 mmol, Intermediate HG), 1-bromo-4-chloro-2-fluoro-3-methyl-benzene (529 mg, 2.37 mmol. CAS #943830-58-8), Cs₂CO₃ (1.93 g, 5.93 mmol), RuPhos (184 mg, 395 μmol) and RuPhos Pd G3 (330 mg, 395 μmol) in dioxane (12 mL) was degassed and purged with N₂ three times. Then the mixture was stirred at 100° C. for 14 hrs under N₂ atmosphere. On completion, the reaction mixture was diluted with H₂O (50 mL) and extracted with EA (50 mL×3). The combined organic layers were washed with brine (150 mL), dried over anhydrous sodium sulfate, filtered and concentrated under reduced pressure to give a residue. The residue was purified by column chromatography (SiO₂. Petroleum ether/Ethyl acetate=1/0 to 3/1) to give the title compound (300 mg, 28% yield) as a white solid. LC-MS (ESI⁺) m/z 497.1 (M+H)⁺.

### Step 2—4-(1-(4-Chloro-2-fluoro-3-methylphenyl)piperidin-4-yl)-5-fluoro-2-methoxyaniline

To a solution of 2-[4-[1-(4-chloro-2-fluoro-3-methyl-phenyl)-4-piperidyl]-5-fluoro-2-methoxy-phenyl]isoindoline-1,3-dione (280 mg, 563 μmol) in EtOH (6 mL) was added NH₂NH₂·H₂O (352 mg. 5.63 mmol, 80% solution) dropwise at 25° C. The mixture was then stirred at 50° C. for 4 hrs. On completion, the reaction mixture was filtered and the filtrate was concentrated under reduced pressure to give the title compound (150 mg) as a light pink oil. LC-MS (ESI⁺) m/z 367.1 (M+H)⁺.

### 3-((4-(1-(4-Chloro-2-fluoro-3-methylphenyl)-1,2,3,6-tetrahydropyridin-4-yl)-5-fluoro-2-methoxyphenyl)amino)piperidine-2,6-dione (Intermediate IX)

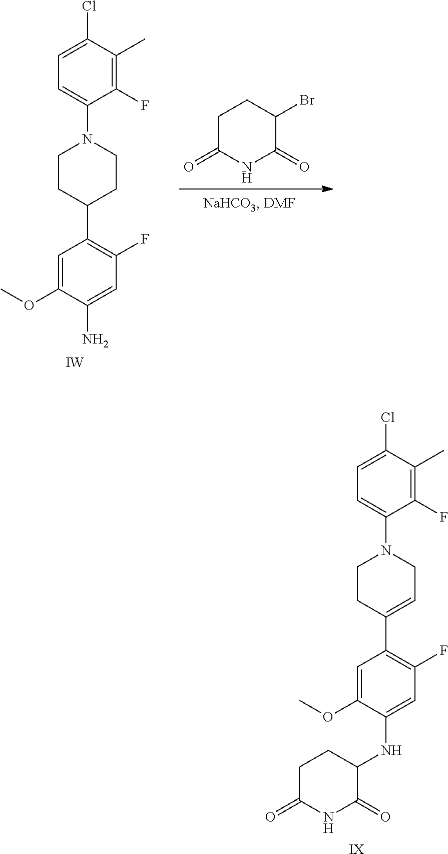

To a solution of 4-[1-(4-chloro-2-fluoro-3-methyl-phenyl)-4-piperidyl]-5-fluoro-2-methoxy-aniline (150 mg, 408 μmol, Intermediate IW) and 3-bromopiperidine-2,6-dione (235 mg, 1.23 mmol, CAS #62595-74-8) in DMF (5 mL) was added NaHCO₃ (171 mg. 2.04 mmol). The mixture was stirred at 80° C. for 24 hrs. On completion, the reaction mixture was diluted with H₂O (20 mL) and extracted with EA (25 mL×2). The combined organic layers were washed with brine (50 mL), dried over anhydrous sodium sulfate, filtered and concentrated under reduced pressure to give a residue. The residue was purified by prep-HPLC (0.1% FA condition) to give a title compound (70 mg, 34% yield) as a white solid. LC-MS (ESI⁺) m/z 478.2 (M+H)⁺.

### Tert-butyl 4-(4-chloro-3-fluorophenyl)-3,3-difluoropiperidine-1-carboxylate (Intermediate IY)

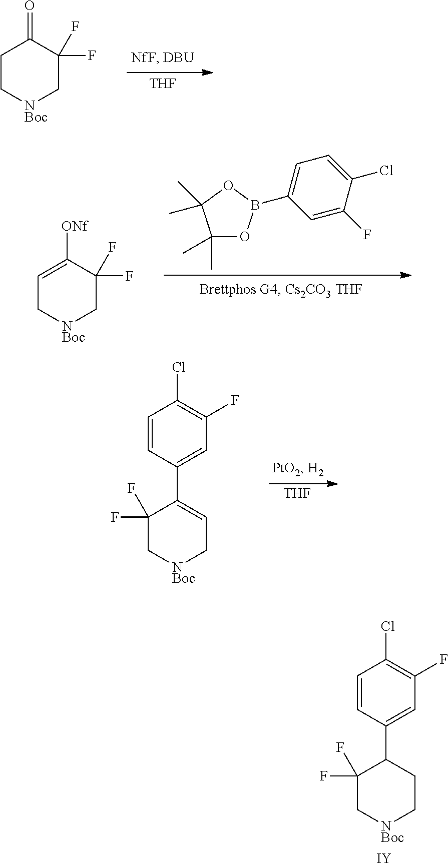

### Step 1—Tert-butyl 3,3-difluoro-4-(((perfluorobutyl)sulfonyl)oxy)-3,6-dihydropyridine-1(2H)-carboxylate

To a solution of tert-butyl 3,3-difluoro-4-oxo-piperidine-1-carboxylate (45.0 g, 191 mmol) in THF (500 mL) was added DBU (87.3 g, 574 mmol) and 1,1,2,2,3,3,4,4,4-nonafluorobutane-1-sulfonyl fluoride (173 g, 574 mmol). The mixture was then stirred at −30° C. for 1 hr. On completion, the residue was diluted with water (500 mL) and extracted with EA (300 mL×3). The combined organic layers were washed with brine (100 mL×3), dried over Na₂SO₄, filtered and concentrated under reduced pressure to give a residue. The residue was purified by column chromatography (SiO₂, Petroleum ether/Ethyl acetate=1/0 to 20/1) to give a residue to give the title compound (69 g, 70% yield) as a white solid. ¹H NMR (400 MHz, CDCl₃-d) δ=6.32-6.14 (m, 1H), 4.25-4.11 (m, 2H), 4.00-3.81 (m, 211), 1.44 (s, 9H).

### Step 2—Tert-butyl 4-(4-chloro-3-fluorophenyl)-3,3-difluoro-3,6-dihydropyridine-1 (2H)-carboxylate

To a solution of tert-butyl 3,3-difluoro-4-(1,1,2,2,3,3,4,4,4-nonafluorobutylsulfonyloxy)-2,6-dihydropyridine-1-carboxylate (20.0 g, 38.6 mmol) in THF (200 mL) was added 2-(4-chloro-3-fluoro-phenyl)-4,4,5,5-tetramethyl-1,3,2-dioxaborolane (11.9 g, 46.3 mmol), Cs₂CO₃ (37.7 g, 116 mmol) and BrettPhos Pd G4 (3.50 g, 3.80 mmol). The mixture was then stirred at 60° C. for 2 hrs. On completion, the reaction mixture was diluted with water (1000 mL) and extracted with EA (1000 mL×3). The combined organic layers were washed with brine (500 mL×2), dried over Na₂SO₄, filtered and concentrated under reduced pressure to give a residue. The residue was purified by column chromatography (SiO₂, Petroleum ether/Ethyl acetate=40/1 to 10/1) to give the title compound (2 g, 15% yield) as a white solid. ¹H NMR (400 MHz, CDCl₃-d) δ=7.32 (t, J=8.0 Hz, 1H), 7.20-7.16 (m, 1H), 7.12 (d, J=8.3 Hz, 1H), 6.24 (s, 1H), 4.12 (s, 2H), 3.88 (t, J=11.1 Hz, 2H), 1.44 (s, 9H).

### Step 3—Tert-butyl 4-(4-chloro-3-fluorophenyl)-3,3-difluoropiperidine-1-carboxylate

A mixture of tert-butyl 4-(4-chloro-3-fluoro-phenyl)-3,3-difluoro-2,6-dihydropyridine-1-carboxylate (1.8 g, 5.1 mmol). Pd/C (918 mg, 5.1 mmol. 60 wt %) in THE (5 mL) was degassed and purged with H₂ 3 times. Then the mixture was stirred at 25° C. for 24 hrs under H₂ atmosphere. On completion, the reaction mixture was filtered and concentrated under reduced pressure to give the title compound (1.6 g, 88% yield) as a white solid.

### Tert-butyl (S)-4-(4-(2-(dimethylcarbamoyl)-7-fluoro-6-(1-isobutyryl-1,2,5,6-tetrahydropyridin-3-yl)-1H-indol-4-yl)-3-fluorophenyl)-3,3-difluoropiperidine-1-carboxylate (Intermediate IZ) and tert-butyl (R)-4-(4-(2-(dimethylcarbamoyl)-7-fluoro-6-(1-isobutyryl-1,2,5,6-tetrahydropyridin-3-yl)-1H-indol-4-yl)-3-fluorophenyl)-3.3 difluoropiperidine-1-carboxylate (Intermediate JA)

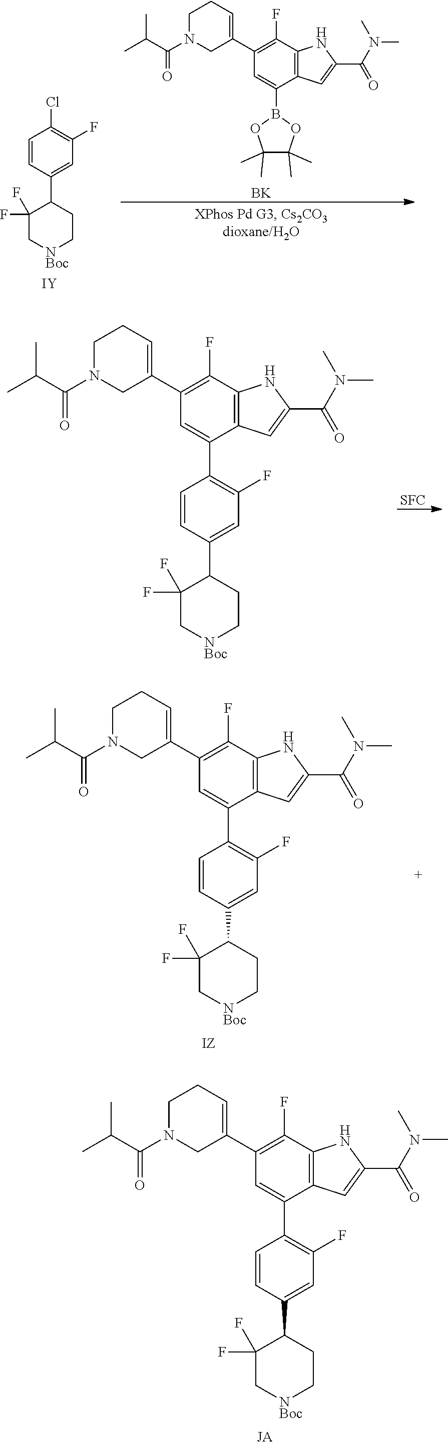

### Step 1—Tert-butyl 4-(4-(2-(dimethylcarbamoyl)-7-fluoro-6-(1-isobutyryl-1,2,5,6-tetrahydropyridin-3-yl)-1H-indol-4-yl)-3-fluorophenyl)-3,3-difluoropiperidine-1-carboxylate

To a solution of tert-butyl 4-(4-chloro-3-fluoro-phenyl)-3,3-difluoro-piperidine-1-carboxylate (1.30 g, 3.70 mmol, Intermediate IY) in dioxane (20 mL) and H₂O (5 mL) was added 7-fluoro-N,N-dimethyl-6-[1-(2-methylpropanoyl)-3,6-dihydro-2H-pyridin-5-yl]-4-(4,4,5,5-tetramethyl-1,3,2-dioxaborolan-2-yl)-1H-indole-2-carboxamide (1.80 g, 3.70 mmol, Intermediate BK), XPhos Pd G3 (315 mg, 372 μmol) and Cs₂CO₃ (3.60 g, 11.1 mmol). The mixture was then stirred at 80° C. for 1 hr. On completion, the mixture was purified by prep-HPLC (0.1% FA condition) to give the title compound (3 g, 49% yield, FA) as a white solid. LC-MS (ESI⁺) m/z 671.4 (M+H)⁺.

### Step 2—Tert-butyl (S)-4-(4-(2-(dimethylcarbamoyl)-7-fluoro-6-(1-isobutyryl-1,2,5,6-tetrahydropyridin-3-yl)-1H-indol-4-yl)-3-fluorophenyl)-3,3-difluoropiperidine-1-carboxylate

Tert-butyl 4-(4-(2-(dimethylcarbamoyl)-7-fluoro-6-(1-isobutyryl-1,2,5,6-tetrahydropyridin-3-yl)-1H-indol-4-yl)-3-fluorophenyl)-3.3 difluoropiperidine-1-carboxylate was separated by SFC (column: DAICEL CHIRALPAK IA (250 mm*30 mm·10 um); mobile phase: [A: Hexane, B: IPA]; B %:30%, isocratic elution mode) to give the first eluting isomer tert-butyl (S)-4-(4-(2-(dimethylcarbamoyl)-7-fluoro-6-(1-isobutyryl-1,2,5,6-tetrahydropyridin-3-yl)-1H-indol-4-yl)-3-fluorophenyl)-3,3-difluoropiperidine-1-carboxylate (300 mg, 38% yield) and the second eluting isomer (R)-4-(4-(2-(dimethylcarbamoyl)-7-fluoro-6-(1-isobutyryl-1,2,5,6-tetrahydropyridin-3-yl)-1H-indol-4-yl)-3-fluorophenyl)-3.3 difluoropiperidine-1-carboxylate (300 mg, 37.1% yield, 99% purity) as a white solids. LC-MS (ESI+) m/z 671.4 (M+H)⁺ for both isomers. The absolute stereochemistry of the enantiomers was assigned arbitrarily.

### (S)-4-(4-(3,3-difluoropiperidin-4-yl)-2-fluorophenyl)-7-fluoro-6-(1-isobutyryl-1,2,5,6-tetrahydropyridin-3-yl)-N,N-dimethyl-1H-indole-2-carboxamide (Intermediate JB)

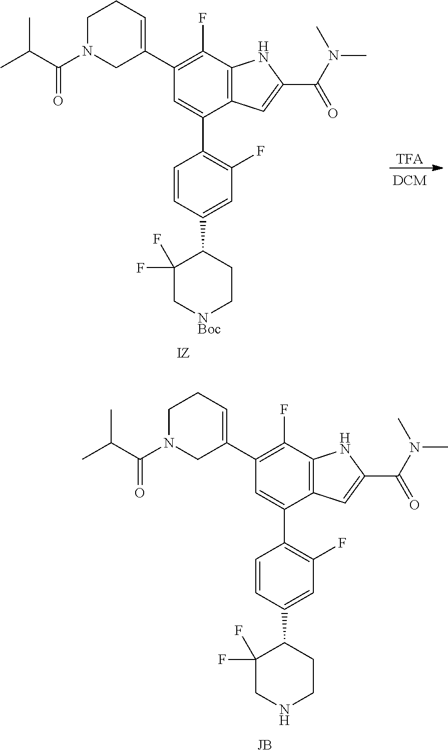

To a solution of tert-butyl (4S)-4-[4-[2-(dimethylcarbamoyl)-7-fluoro-6-[1-(2-methylpropanoyl)-3,6-dihydro-2H-pyridin-5-yl]-1H-indol-4-yl]-3-fluoro-phenyl]-3,3-difluoro-piperidine-1-carboxylate (270 mg, 403 μmol, Intermediate IZ) in DCM (6 mL) was added TFA (3.07 g, 26.9 mmol). The mixture was then stirred at 25° C. for 1 hr. On completion, the mixture was purified by prep-HPLC (0.1% FA condition) to give the title compound (190 mg, 73% yield, FA) as a white solid. LC-MS (ESI⁺) m/z 571.3 (M+H)⁺.

### (R)-1-(4-bromo-2,5-difluorophenyl)-2-methylpiperazine (Intermediate JC)

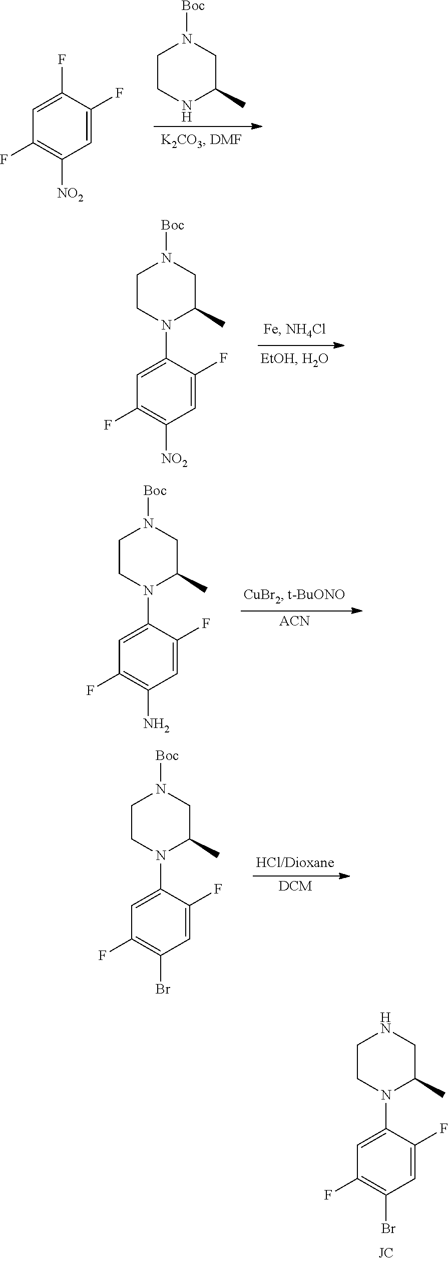

### Step 1—(R)-tert-butyl 4-(2,5-difluoro-4-nitrophenyl)-3-methylpiperazine-1-carboxylate

A solution of 1,2,4-trifluoro-5-nitrobenzene (2.00 g, 11.2 mmol, CAS #2105-61-5), (R)-tert-butyl 3-methylpiperazine-1-carboxylate (2.26 g, 11.2 mmol, CAS #163765-44-4), K₂CO₃ (4.68 g, 33.8 mmol) in DMF (40 mL) was stirred at 80° C. for 2 hrs. On completion, the reaction mixture was quenched with water (40 mL) and extracted with ethyl acetate (3×40 mL). The combined organic layers were washed with brine (50 mL) and dried over anhydrous sodium sulfate, filtered and the filtrate was concentrated in vacuo to give the crude residue. The residue was purified by column chromatography (SiO₂, Petroleum ether:Ethyl acetate=100/1 to 10/1) to give the title compound (4.00 g, 99% yield) as a yellow oil. ¹H NMR (400 MHz, DMSO-d₆) δ=8.01 (dd, J=7.2, 13.6 Hz, 1H), 7.10 (dd, J=7.6, 14.4 Hz, 1H), 4.13-4.02 (m, 1H), 3.91 (dd, J=2.8, 6.8 Hz, 1H), 3.78-3.69 (m, 2H), 3.38 (d, J=3.2 Hz, 1H), 3.28 (d, J=3.6 Hz, 1H), 3.26-3.17 (m, 1H), 3.10-2.98 (m, 1H), 1.43 (s, 9H), 1.21-1.11 (m, 1H), 1.08 (d, J=6.4 Hz, 3H).

### Step 2—(R)-tert-butyl 4-(4-amino-2.5 difluorophenyl)-3-methylpiperazine-1-carboxylate

A solution of tert-butyl (3R)-4-(2,5-difluoro-4-nitro-phenyl)-3-methyl-piperazine-1-carboxylate (3.70 g, 10.3 mmol), NH4Cl (5.54 g, 103 mmol), Fe (5.78 g, 103 mmol) in EtOH (60 mL) and H₂O (15 mL) was stirred at 80° C. for 2 hrs. On completion, the reaction mixture was filtered and the filtrate was concentrated in vacuo to give the crude residue. The residue was purified by column chromatography (SiO₂, Petroleum ether:Ethyl acetate=100/1 to 50/1) to give the title compound (1.50 g, 40% yield) as a yellow oil. LC-MS (ESI⁺) m/z 328.0 (M+H)⁺.

### Step 3—(R)-tert-butyl 4-(4-bromo-2,5-difluorophenyl)-3-methylpiperazine-1-carboxylate

A solution of CuBr₂ (573 mg, 2.57 mmol), t-BuONO (330 mg, 3.21 mmol), and tert-butyl (R)-tert-butyl 4-(4-amino-2,5-difluorophenyl)-3-methylpiperazine-1-carboxylate (700 mg, 2.14 mmol) in ACN (2 mL) was stirred at 25° C. for 2 hrs. On completion, the reaction mixture was quenched with water (20 mL) and extracted with dichloromethane (3×20 mL). The combined organic layers were washed with brine (20 mL) and dried over anhydrous sodium sulfate, filtered and the filtrate was concentrated in vacuo to give the crude residue. The residue was purified by column chromatography (SiO₂, Petroleum ether:Ethyl acetate=100/1 to 10/1) to give the title compound (400 mg, 43% yield) as a yellow oil. LC-MS (ESI⁺) m/z 337.2 (M−55)⁻,

### Step 4—(R)-1-(4-bromo-2,5-difluorophenyl)-2-methylpiperazine

A solution of (R)-tert-butyl 4-(4-bromo-2,5-difluorophenyl)-3-methylpiperazine-1-carboxylate (200 mg, 511 μmol) in DCM (2 mL) and HCl/dioxane (1 mL) was stirred at 25° C. for 2 hrs. On completion, the reaction mixture was concentrated in vacuo to give the title compound (160 mg) as a yellow solid. LC-MS (ESI⁺) m/z 290.8 (M+H)⁺.

### 3-(4-(2-((R)-4-(4-bromo-2,5-difluorophenyl)-3-methylpiperazin-1-yl)ethyl)-3-methyl-2-oxo-2,3-dihydro-1H-benzo[d]imidazol-1-yl)piperidine-2,6-dione (Intermediate JD)

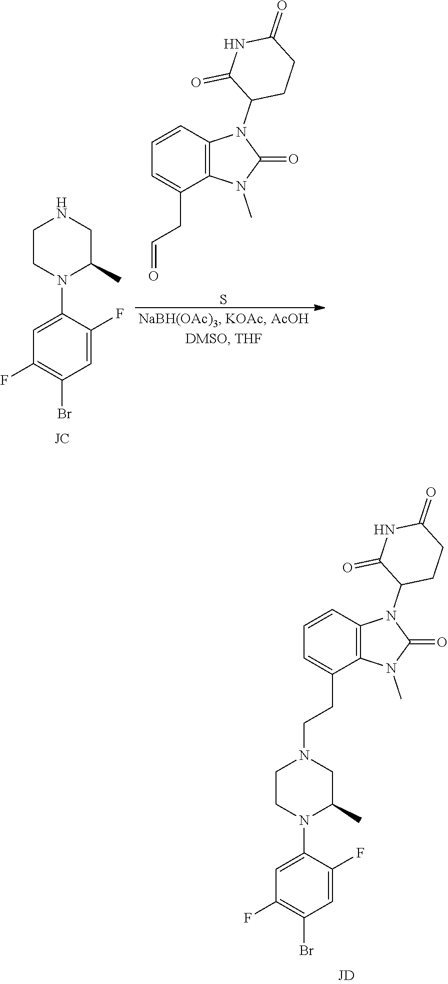

A solution of (R)-1-(4-bromo-2,5-difluorophenyl)-2-methylpiperazine (70.0 mg, 240 μmol, Intermediate JC), 2-(1-(2,6-dioxopiperidin-3-yl)-3-methyl-2-oxo-2,3-dihydro-1H-benzo[d]imidazol-4-yl)acetaldehyde (115 mg, 384 μmol, Intermediate S), KOAc (47.1 mg, 480 μmol), and NaBH(OAc)₃ (152 mg, 721 μmol) in DMSO (1 mL) and THF (1 mL) was stirred at 25° C. for 2 hrs. On completion, the reaction mixture was quenched with water (10 mL) and extracted with ethyl acetate (4×10 mL). The combined organic layers were washed with brine (20 mL) and dried over anhydrous sodium sulfate, filtered and the filtrate was concentrated in vacuo to give the crude residue. The residue was purified by column chromatography (SiO₂, Petroleum ether:Ethyl acetate=100/1 to 0/1) to give the tittle compound (60.0 mg, 41% yield) as a yellow solid. LC-MS (ESI⁺)/z 576.0 (M+H)⁺.

### 6-(1-(3-(1H-pyrazol-1-yl)propanoyl)-1.2.5.6 tetrahydropyridin-3-yl)-4-chloro-7-fluoro-N,N-dimethyl-1H-indole-2-carboxamide (Intermediate JE)

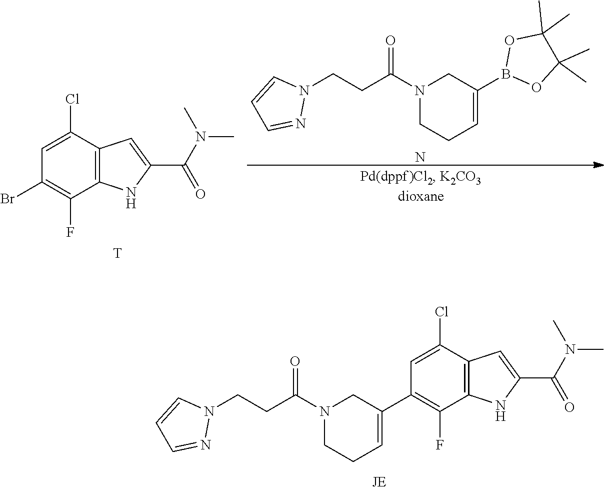

6-(1-(3-(1H-pyrazol-1-yl)propanoyl)-1,2,5,6-tetrahydropyridin-3-yl)-4-chloro-7-fluoro-N,N-dimethyl-1H-indole-2-carboxamide was synthesized as described in Step 1 of Intermediate AR,

### 3-(2,6-difluoro-4-(4-(4-(4,4,5,5-tetramethyl-1,3,2-dioxaborolan-2-yl)phenyl)piperidin-1-yl)phenyl)piperidine-2,6-dione (Intermediate JF)

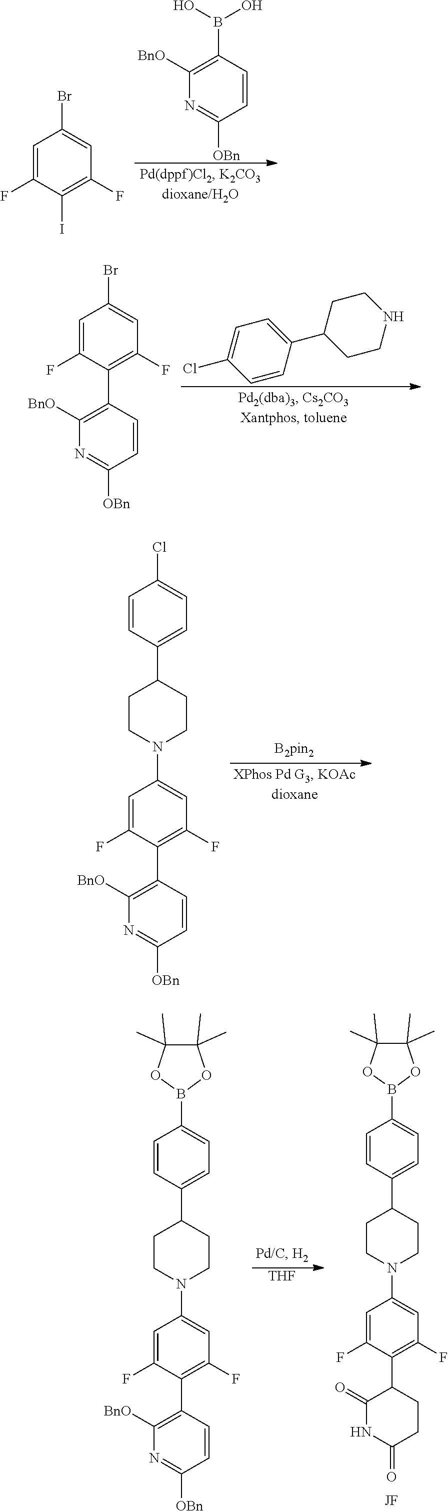

### Step 1—2,6-Bis(benzyloxy)-3-(4-bromo-2,6-difluorophenyl)pyridine

A mixture of 5-bromo-1,3-difluoro-2-iodo-benzene (4.00 g, 12.5 mmol, CAS #160976-02-3), (2,6-dibenzyloxy-3-pyridyl)boronic acid (4.20 g, 12.5 mmol, CAS #2096339-92-1), Pd(dppf)Cl₂·CH₂Cl₂ (1.02 g, 1.25 mmol), K₂CO₃ (5.20 g, 37.6 mmol) in dioxane (40 mL) and H₂O (10 mL) was degassed and purged with N₂ 3 times. Then the mixture was stirred at 60° C. for 2 hrs under N₂ atmosphere. On completion, the reaction mixture was concentrated under reduced pressure to remove solvent. The residue was purified by column chromatography (SiO₂, Petroleum ether:Ethyl acetate=50/1) to give the title compound (2.90 g, 30% yield) as a yellow solid. LC-MS (ESI⁺) m/z 482.0 (M+H)⁺.

### Step 2—2,6-Bis(bcnzyloxX)-3-(4-(4-(4-chlorophenyl)piperidin-1-yl)-2,6-difluorophenyl)pyridine

A mixture of 2.6 dibenzyloxy-3-(4-bromo-2,6-difluoro-phenyl)pyridine (2.70 g, 5.60 mmol), 4-(4-chlorophenyl)piperidine (1.10 g, 5.60 mmol, CAS #26905-02-2), Pd₂(dba)₃ (256 mg, 280 μmol), Cs₂CO₃ (5.47 g, 16.8 mmol) and Xantphos (324 mg, 560 μmol) in toluene (25 mL) was degassed and purged with N₂ 3 times. Then the mixture was stirred at 100° C. for 12 hrs under N₂ atmosphere. On completion, the reaction mixture was concentrated under N₂ to remove solvent. The crude residue was purified by column chromatography (SiO₂, Petroleum ether:Ethyl acetate=30/1) to give the title compound (1.20 g, 31% yield) as a yellow solid. LC-MS (ESI⁺) m/z 597.1 (M+H)⁺.

### Step 3—2.6 Bis(benzyloxy)-3-(2,6-difluoro-4-(4-(4-(4,4,5,5-tetramethyl-1.3.2 dioxaborolan-2-yl)phenyl)piperidin-1-yl)phenyl)pyridine

A mixture of 2,6-dibenzyloxy-3-[4-[4-(4-chlorophenyl)-1-piperidyl]-2,6-difluoro-phenyl]pyridine (1.00 g, 1.67 mmol), 4,4,5,5-tetramethyl-2-(4,4,5,5-tetramethyl-1,3,2-dioxaborolan-2-yl)-1,3,2-dioxaborolane (851 mg, 3.35 mmol), KOAc (493 mg, 5.02 mmol), and XPhos Pd G3 (213 mg. 251 μmol) in dioxane (10 mL) was degassed and purged with N₂ 3 times. Then the mixture was stirred at 80° C. for 1 hr under N₂ atmosphere. On completion, the reaction mixture was filtered to give a residue. The crude residue was purified by column chromatography (SiO₂, Petroleum ether/Ethyl acetate=20/1 to DCM/Ethyl acetate=1/1) to give the title compound (1.00 g, 75% yield) as a white solid. LC-MS (ESI⁺) m/z 689.3 (M+H)⁺.

### Step 4—3-(2.6 Difluoro-4-(4-(4-(4,4,5,5-tetramethyl-1,3,2-dioxaborolan-2-yl)phenyl)piperidin-1-yl)phenyl)piperidine-2,6-dione

A mixture of 2,6-dibenzyloxy-3-[2,6-difluoro-4-[4-[4-(4,4,5,5-tetramethyl-1,3,2-dioxaborolan-2-yl)phenyl]-1-piperidyl]phenyl]pyridine (500 mg, 726 μmol), Pd/C (500 mg, 470 μmol, 10 wt) in THF (20 mL) was degassed and purged with H₂ 3 times. Then the mixture was stirred at 30° C. for 12 hrs under H₂ atmosphere. On completion, the reaction mixture was filtered and concentrated under reduced pressure to give a residue. The crude residue was purified by column chromatography (SiO₂, Petroleum ether/Ethyl acetate=1/1 to DCM/Ethyl acetate=1/1) to give the title compound (260 mg, 62% yield) as a white solid. LC-MS (ESI⁺) m/z 511.2 (M+H)⁺.

### 4-(4-Chloro-3-methoxyphenethyl)piperidine (Intermediate JG)

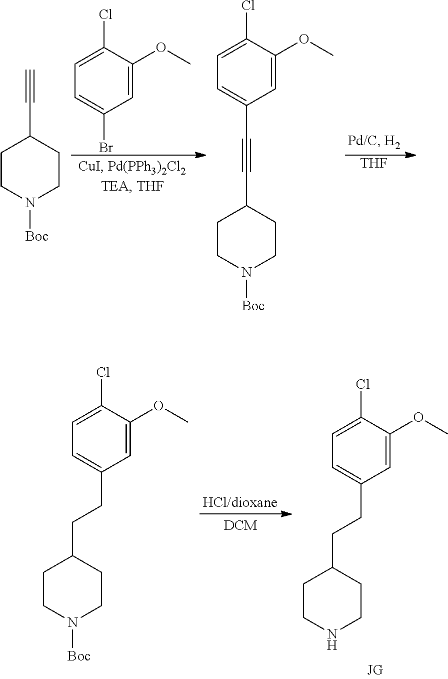

### Step 1—Tert-butyl 4-((4-chloro-3-methoxyphenyl)ethynyl)piperidine-1-carboxylate

A mixture of tert-butyl 4-ethynylpiperidine-1-carboxylate (5 g. 20 mmol, CAS #287192-97-6), 4-bromo-1-chloro-2-methoxybenzene (5.29 g, 23.8 mmol, CAS #16817-43-9), TEA (9.67 g, 95.5 mmol) and Pd(PPh₃)₂Cl₂ (838 mg, 1.19 mmol) in DMF (40 mL) was degassed and purged with N₂ three times. Next, the mixture was stirred at 20° C. for 10 min, and then CuI (227 mg, 1.19 mmol) in DMF (10 mL) was added. Then the mixture was stirred at 80° C. for 10 hrs under N₂ atmosphere. On completion, the mixture was quenched with water (200 mL) and extracted with ethyl acetate (2×100 mL). The combined organic layers were washed with brine (200 mL) and dried over anhydrous sodium sulfate, filtered and the filtrate was concentrated in vacuo to give the crude residue. The residue was purified by column chromatography (SiO₂, Petroleum ether:Ethyl acetate=1/0 to 10/1) to give the title compound (8 g, 96% yield) as a white solid. LC-MS (ESI⁺) m/z 249.9 (M−99)⁺.

### Step 2—Tert-butyl 4-(4-chloro-3-methoxyphenethyl)piperidine-1-carboxylate

To a solution of tert-butyl 4-((4-chloro-3-methoxyphenyl)ethynyl)piperidine-1-carboxylate (7 g, 20 mmol) in THF (40 mL) was added Pd/C (10 wt %, 7 g) under N₂ atmosphere. The suspension was degassed and purged with H₂ 3 times. Then the mixture was stirred under H₂ (15 Psi) at 25° C. for 2 hrs. On completion, the mixture was diluted with water (10 mL) and extracted with dichloromethane (2×10 mL). The combined organic layers were washed with brine (20 mL) and dried over anhydrous sodium sulfate, filtered and the filtrate was filtered and the filtrate was concentrated in vacuo to give the title compound (8 g) as yellow oil. LC-MS (ESI⁺) m/z 298.2 (M−55)⁺.

### Step 3—4-(4-Chloro-3-methoxyphenethyl)piperidine

To a solution of tert-butyl 4-(4-chloro-3-methoxyphenethyl)piperidine-1-carboxylate (7 g, 20 mmol) in DCM (60 mL) was added HCl/dioxane (4 M, 10 mL). The mixture was then stirred at 25° C. for 2 hrs. On completion, the mixture was concentrated in vacuo and purified by reversed-phase HPLC (0.1% FA condition) to give the title compound (5 g, 99% yield) as a colorless oil. LC-MS (ESI⁺) m/z 254.0 (M+H)⁺.

### 3-(3-Fluoro-4-(4-(3-methoxy-4-(4,4,5,5-tetramethyl-1,3,2-dioxaborolan-2-yl)phenethyl)piperidin-1-yl)phenyl)piperidine-2,6-dione (Intermediate JH)

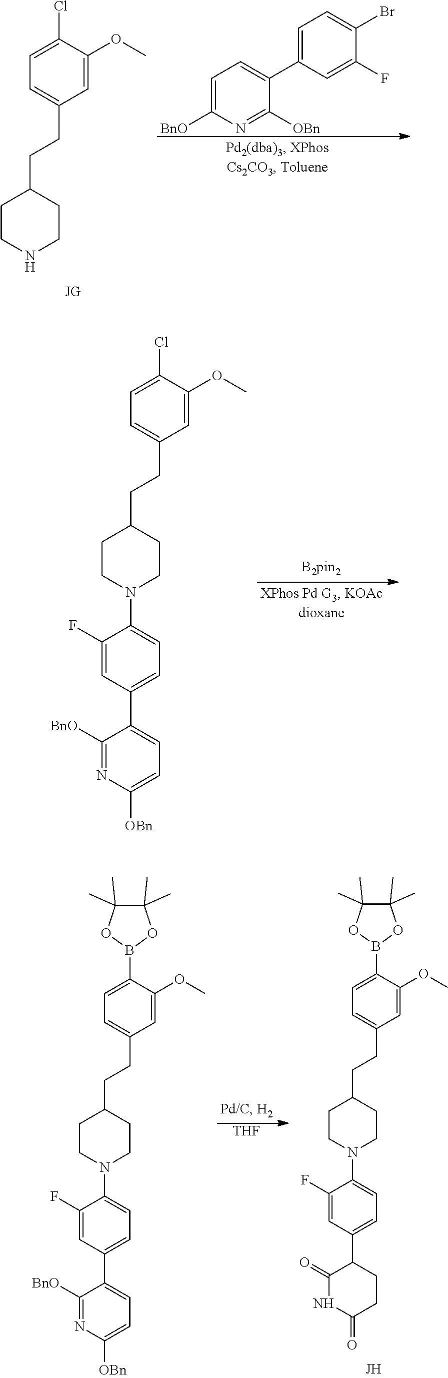 [SMILES: COc1cc(CCC2CCNCC2)ccc1Cl ; Fc1cc(-c2ccc(OCc3ccccc3)nc2OCc2ccccc2)ccc1Br ; COc1cc(CCC2CCN(c3ccc(-c4ccc(OCc5ccccc5)nc4OCc4ccccc4)cc3F)CC2)ccc1Cl ; "THF" ; COc1cc(CCC2CCN(c3ccc(-c4ccc(OCc5ccccc5)nc4OCc4ccccc4)cc3F)CC2)ccc1B1OC(C)(C)C(C)(C)O1 ; COc1cc(CCC2CCN(c3ccc(C4CCC(=O)NC4=O)cc3F)CC2)ccc1B1OC(C)(C)C(C)(C)O1]

### Step 1—2,6-Bis(benzyloxy)-3-(4-(4-(4-chloro-3-methoxyphenethyl)piperidin-1-yl)-3-fluorophenyl)pyridine

A mixture of 4-(4-chloro-3-methoxyphenethyl)piperidine (500 mg, 1.97 mmol, Intermediate JG), 2,6-bis(benzyloxy)-3-(4-bromo-3-fluorophenyl)pyridine (914 mg, 1.97 mmol, CAS #2902652-28-0), Pd₂(dba)₃ (90.2 mg, 98.5 μmol), XPhos (93.9 mg, 197 μmol) and Cs₂CO₃ (1.93 g, 5.91 mmol) in toluene (10 mL) was degassed and purged with N₂ three times. Then the mixture was stirred at 100° C. for 2 hrs under N₂ atmosphere. On completion, the mixture was quenched with water (50 mL) and extracted with dichloromethane (2×50 mL). The combined organic layers were washed with brine (100 mL) and dried over anhydrous sodium sulfate, filtered and the filtrate was concentrated in vacuo to give the crude residue. The residue was purified by column chromatography (SiO₂, Petroleum ether:Ethyl acetate=1/0 to 3/1) to give the title compound (750 mg, 60% yield) as yellow oil. LC-MS (ESI⁺) m/z 637.1 (M+H)⁺.

### Step 2—2,6-Bis(benzyloxy)-3-(3-fluoro-4-(4-(3-methoxy-4-(4,4,5,5-tetramethyl-1,3,2-dioxaborolan-2-yl)phenethyl)piperidin-1-yl)phenyl)pyridine

A mixture of 2,6-bis(benzyloxy)-3-(4-(4-(4-chloro-3-methoxyphenethyl)piperidin-1-yl)-3-fluorophenyl)pyridine (570 mg, 894 μmol), 4,4,5,5-tetramethyl-2-(4,4,5,5-tetramethyl-1,3,2-dioxaborolan-2-yl)-1,3,2-dioxaborolane (340 mg, 1.34 mmol, CAS #73183-34-3), XPhos Pd G3 (75.7 mg, 89.4 μmol), and AcOK (263 mg, 2.68 mmol) in dioxane (10 mL) was degassed and purged with N₂ three times. Then the mixture was stirred at 80° C. for 12 hrs under N₂ atmosphere. On completion, the mixture was quenched with water (10 mL) and extracted with dichloromethane (2×10 mL). The combined organic layers were washed with brine (20 mL) and dried over anhydrous sodium sulfate, filtered and the filtrate was concentrated in vacuo to give the crude residue. The crude residue was purified by column chromatography (SiO₂, Petroleum ether:Ethyl acetate=1/0 to 10/1) to give the title compound (350 mg, 54% yield) as a yellow solid. LC-MS (ESI⁺) m/z 729.4 (M+H)⁺.

### Step 3—3-(3-Fluoro-4-(4-(3-methoxy-4-(4,4,5,5-tetramethyl-1,3,2-dioxaborolan-2-yl)phenethyl)piperidin-1-yl)phenyl)piperidine-2,6-dione

To a solution of 2,6-bis(benzyloxy)-3-(3-fluoro-4-(4-(3-methoxy-4-(4,4,5,5-tetramethyl-1,3,2-dioxaborolan-2-yl)phenethyl)piperidin-1-yl)phenyl)pyridine (350 mg, 480 μmol) in THE (20 mL) was added Pd/C (10 wt %) under N₂ atmosphere. The suspension was degassed and purged with H₂ three times. The mixture was then stirred under H₂ (15 psi) at 25° C. for 12 hrs. On completion, the mixture was filtered and the filtrate was concentrated in vacuo and purified by prep-HPLC (0.1% FA condition)) to give the title compound (150 mg, 57% yield) as a white solid. LC-MS (ESF) m/z 551.3 (M+H)⁺.

### Step 1—6-(1-(3-(1H-1,2,3-triazol-1-yl)propanoyl)-1,2,5,6-tetrahydropyridin-3-yl)-4-chloro-7-fluoro-N,N-dimethyl-1H-indole-2-carboxamide (Intermediate JI)

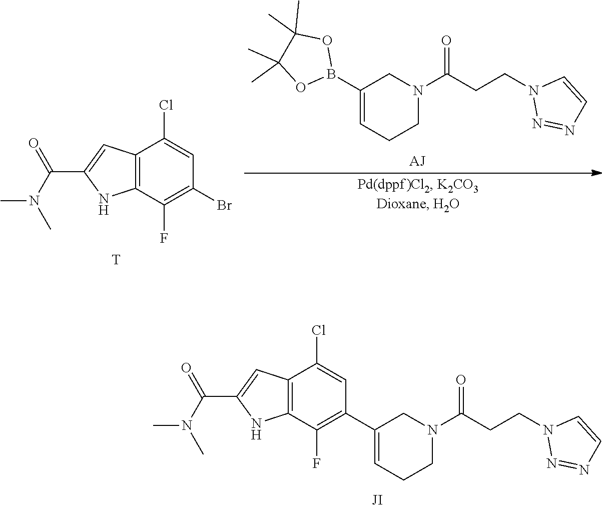

6-(1-(3-(1H-1,2,3-triazol-1-yl)propanoyl)-1,2,5,6-tetrahydropyridin-3-yl)-4-chloro-7-fluoro-N,N-dimethyl-1H-indole-2-carboxamide was synthesized as described in Step 1 of Intermediate AK.

### 4-(4-Chloro-3-methylphenethyl)piperidine (Intermediate JJ)

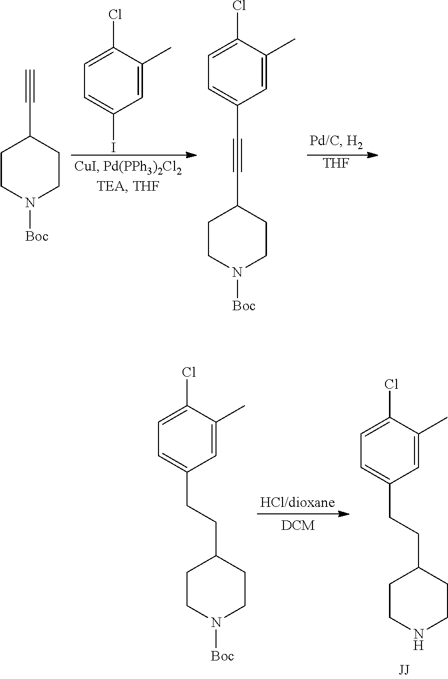

### Step 1—Tert-Butyl 4-((4-chloro-3-methylphenyl)ethynyl)piperidine-1-carboxylate

A mixture of tert-butyl 4-ethynylpiperidine-1-carboxylate (5 g, 20 mmol, CAS #287192-97-6). 1-chloro-4-iodo-2-methyl-benzene (6.03 g, 23.9 mmol, CAS #116632-41-8). CuI (228 mg, 1.19 mmol), TEA (9.67 g, 95.6 mmol) and Pd(PPh₃)₂Cl₂ (838 mg, 1.19 mmol) in THF (110 mL) was degassed and purged with N₂ three times. Then the mixture was stirred at 25° C. for 12 hrs under N₂ atmosphere. On completion, the reaction mixture was concentrated under reduced pressure to give a residue. The residue was purified by column chromatography (SiO₂, Petroleum ether/Ethyl acetate=1/0 to 9/1) to give the title compound (7 g, 87% yield) as a colorless oil. LC-MS (ESI⁺) m/z 278.1 (M−55)⁺; ¹H NMR (400 MHz, DMSO-d₆) δ=7.40 (s, 1H), 7.37 (d, J=8.4 Hz, 1H), 7.22 (d, J=8.4 Hz, 1H), 3.66-3.59 (m, 2H), 3.13 (t, J=9.6 Hz, 2H), 2.29 (s, 3H), 1.86-1.74 (m, 2H), 1.54-1.44 (m, 3H), 1.40 (s, 9H).

### Step 2—Tert-butyl 4-(4-chloro-3-methylphenethyl)piperidine-1-carboxylate

To a solution of tert-butyl 4-[2-(4-chloro-3-methyl-phenyl)ethynyl]piperidine-1-carboxylate (6.8 g, 20.4 mmol) in THF (80 mL) was added Pd/C (3 g, 1.41 mmol, 10 wt %). The suspension was degassed under vacuum and purged with H₂ several times. The mixture was stirred under H₂ (15 psi) at 25° C. for 3 hrs. On completion, the reaction was filtered through kieselguhr carefully, then the filtrate was concentrated to give the title compound (6.3 g) as a light-yellow oil. LC-MS (ESI⁺) n/z 282.1 (M−55)

### Step 3—4-(4-Chloro-3-methylphenethyl)piperidine

To a solution of tert-butyl 4-[2-(4-chloro-3-methyl-phenyl)ethyl]piperidine-1-carboxylate (6.30 g, 18.7 mmol) in HCl/dioxane (2 M, 39.4 mL). The mixture was then stirred at 25° C. for 2 hrs. On completion, the reaction mixture was adjusted to pH of 7 with sat. NaHCO₃ and then concentrated under reduced pressure to give a residue. The residue was purified by prep-HPLC (0.1% FA condition) to give the title compound (2.99 g, 62% yield) as a yellow oil. LC-MS (ESI⁺) m/z 238.1 (M+H); ¹H NMR (400 MHz, DMSO-d₆) δ=8.41 (s, 1H), 7.28 (d, J=8.0 Hz, 1H), 7.18 (s, 1H), 7.04 (dd, J=1.2, 8.4 Hz, 1H), 3.18 (d, J=10.0 Hz, 2H), 2.73 (t, J=11.6 Hz, 2H), 2.53 (t, J=7.2 Hz, 2H), 2.28 (s, 3H), 1.84-1.70 (m, 2H), 1.52-1.42 (m, 3H), 1.37-1.21 (in, 2H).

### 3-(3-Fluoro-4-(4-(3-methyl-4-(4,4,5,5-tetramethyl-1,3,2-dioxaborolan-2-yl)phenethyl)piperidin-1-yl)phenyl)piperidine-2,6-dione (Intermediate JK)

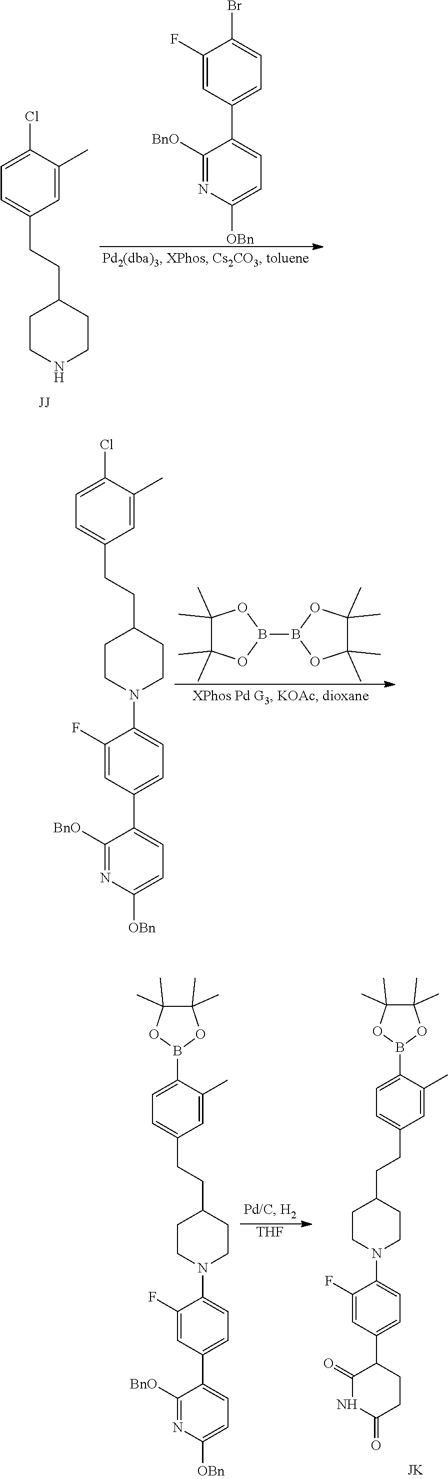

### Step 1—2,6-Bis(benzyloxy)-3-(4-(4-(4-chloro-3-methylphenethyl)piperidin-1-yl)-3-fluorophenyl)pyridine

A mixture of 4-[2-(4-chloro-3-methyl-phenyl)ethyl]piperidine (768 mg, 3.23 mmol, Intermediate JJ), 2,6-dibenzyloxy-3-(4-bromo-3-fluoro-phenyl)pyridine (1.5 g, 3.2 mmol), Pd₂(dba)₃ (148 mg, 162 μmol), XPhos (154 mg, 323 μmol) and Cs₂CO₃ (3.16 g, 9.69 mmol) in toluene (30 mL) was degassed and purged with N₂ three times. Then the mixture was stirred at 100° C. for 12 hrs under N₂ atmosphere. On completion, the reaction mixture was concentrated under reduced pressure to give a residue. The residue was purified by column chromatography (SiO₂, Petroleum ether/Ethyl acetate=1/0 to 19/1) to give the title compound (860 mg, 42% yield) as a yellow oil. LC-MS (ESI⁺) m/z 621.2 (M+H)⁺.

### Step 2—2,6-Bis(benzyloxy)-3-(3-fluoro-4-(4-(3-methyl-4-(4,4,5,5-tetramethyl-1,3,2-dioxaborolan-2-yl)phenethyl)piperidin-1-yl)phenyl)pyridine

A mixture of 2,6-dibenzyloxy-3-[4-[4-[2-(4-chloro-3-methyl-phenyl)ethyl]-1-piperidyl]-3-fluoro-phenyl]pyridine (760 mg, 1.22 mmol). 4,4,5,5-tetramethyl-2-(4,4,5,5-tetramethyl-1,3,2-dioxaborolan-2-yl)-1,3,2-dioxaborolane (777 mg, 3.06 mmol), XPhos Pd G3 (103 mg, 122 μmol) and KOAc (240 mg. 2.45 mmol) in dioxane (20 mL) was degassed and purged with N₂ three 3 times. Then the mixture was stirred at 80° C. for 2 hrs under N₂ atmosphere. On completion, the reaction mixture was concentrated under reduced pressure to give a residue. The residue was purified by column chromatography (SiO₂, Petroleum ether/Ethyl acetate=1/0 to 19/1) give the title compound (300 mg, 34% yield) as a yellow solid; LC-MS (ESI⁺) m/z 713.2 (M+H)⁺; ¹H NMR (400 MHz, CDCl₃-d) δ=7.73-7.68 (in, 1H), 7.59 (d, J=8.0 Hz, 1H), 7.43-7.27 (m, 12H), 7.04-7.00 (m, 2H), 6.96 (t, J=8.8 Hz, 1H), 6.46 (d, J=8.0 Hz, 1H), 5.44 (s, 2H), 5.36 (s, 2H), 3.49 (d, J=11.6 Hz, 2H), 2.71-2.61 (m, 4H), 2.54 (s, 3H), 1.85 (d, J=11.2 Hz, 2H), 1.67-1.58 (m, 2H), 1.48 (dd, J=9.2, 11.6 Hz, 3H), 1.34 (s, 12H).

### Step 3—3-(3-Fluoro-4-(4-(3-methyl-4-(4,4,5,5-tetramethyl-1,3,2-dioxaborolan-2-yl)phenethyl)piperidin-1-yl)phenyl)piperidine-2,6-dione

To a solution of 2,6-dibenzyloxy-3-[3-fluoro-4-[4-[2-[3-methyl-4-(4,4,5,5-tetramethyl-1,3,2-dioxaborolan-2-yl)phenyl]ethyl]-1-piperidyl]phenyl]pyridine (300 mg, 420 μmol) in THE (8 mL) was added Pd/C (300 mg, 281 μmol, 10 wt %) under Ar. The suspension was degassed under vacuum and purged with H₂ several times. The mixture was stirred under H₂ (40 psi) at 30° C. for 12 hrs. On completion, the reaction was filtered through kieselguhr carefully, then the filtrate was concentrated in vacuo to give the title compound (200 mg). LC-MS (ESI+) m/z 535.4 (M+H)⁺.

### 4-(7-(4-Chlorophenyl)-2,7-diazaspiro[3.5]nonan-2-yl)-2-fluorobenzoic acid (Intermediate JL)

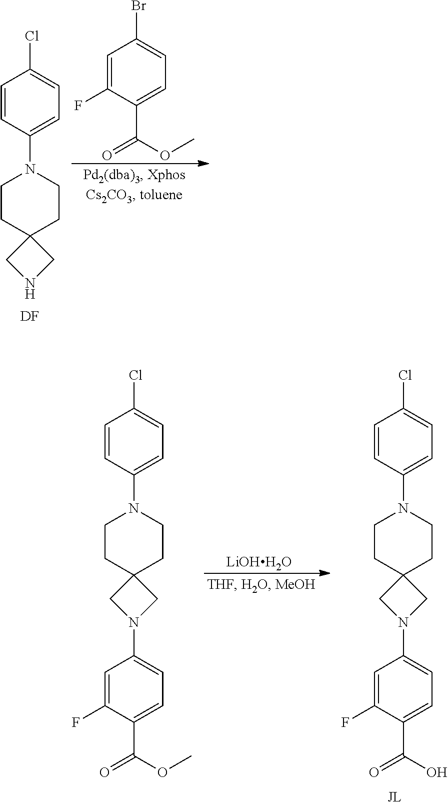

### Step 1—Methyl 4-(7-(4-chlorophenyl)-2,7-diazaspiro[3.5]nonan-2-yl)-2-fluorobenzoate

To a solution of 7-(4-chlorophenyl)-2,7-diazaspiro[3.5]nonane (450 mg, 1.9 mmol, Intermediate DF) in toluene (5 mL) was added methyl 4-bromo-2-fluoro-benzoate (443 mg, 1.9 mmol), (5-diphenylphosphanyl-9,9-dimethyl-xanthen-4-yl)-diphenyl-phosphane (220 mg, 380 μmol), Cs₂CO₃ (1.86 g, 5.70 mmol) and diacetoxypalladium (42.6 mg, 190 μmol). The mixture was then stirred at 90° C. for 2 hrs. On completion, the mixture was purified by prep-HPLC (0.10% FA condition) to give the title compound (400 mg, 29% yield, FA) as a white solid. LC-MS (ESI+) m/z 389.0 (M+H)⁺.

### Step 2—4-(7-(4-Chlorophenyl)-2,7-diazaspiro[3.5]nonan-2-yl)-2-fluorobenzoic acid

To a solution of methyl 4-[7-(4-chlorophenyl)-2,7-diazaspiro[3.5]nonan-2-yl]-2-fluoro-benzoate (250 mg, 643 μmol) in THF (2 mL), H₂O (2 mL) and MeOH (2 mL) was added LiOH·H₂O (270 mg, 6.40 mmol). The mixture was then stirred at 25° C. for 2 hrs. On completion, the mixture was purified by prep-HPLC (0.1% FA condition) to give the title compound (240 mg, 88% yield, FA) as a white solid. LC-MS (ESI+) m/z 375.0 (M+H)⁺.

### 4-(7-(4-Chlorophenyl)-2,7-diazaspiro[3.5]nonan-2-yl)-N-(2,6-dioxopiperidin-3-yl)-2-fluorobenzamide (Intermediate JM)

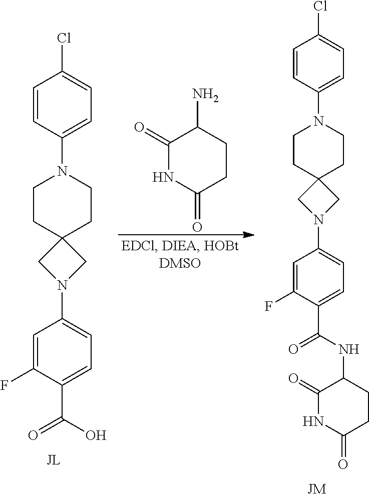 [SMILES: O=C(O)c1ccc(N2CC3(CCN(c4ccc(Cl)cc4)CC3)C2)cc1F ; NC1CCC(=O)NC1=O ; O=C1CCC(NC(=O)c2ccc(N3CC4(CCN(c5ccc(Cl)cc5)CC4)C3)cc2F)C(=O)N1]

To a solution of 4-[7-(4-chlorophenyl)-2,7-diazaspiro[3.5]nonan-2-yl]-2-fluoro-benzoic acid (220 mg, 523 μmol, Intermediate JL) in DMSO (4 mL) was added DIEA (202.6 mg, 1.5 mmol), 3-aminopiperidine-2,6-dione (73.7 mg, 575 μmol), EDCI (251 mg, 1.3 mmol) and HOBt (106 mg, 784 μmol). The mixture was then stirred at 25° C. for 1 hr. On completion, the mixture was purified by prep-HPLC (0.8 g/L NH₄HCO₃ condition) to give the title compound (190 mg, 65% yield, FA) as a white solid. LC-MS (ESI+) m/z 485.1 (M+H)⁺.

### 7-(4-Chloro-2,5-difluorophenyl)-2,7-diazaspiro[3.5]nonane (Intermediate JN)

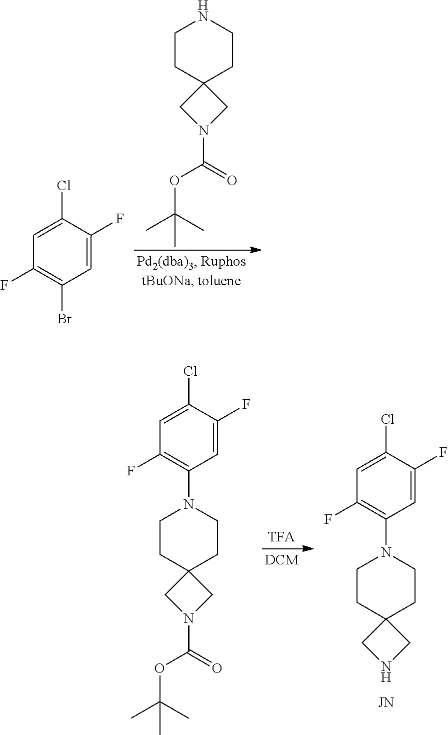

### Step 1—Tert-butyl 7-(4-chloro-2,5-difluorophenyl)-2,7-diazaspiro[3.5]nonane-2-carboxylate

A mixture of 1-bromo-4-chloro-2,5-difluorobenzene (4 g, 20 mmol, CAS #172921-33-4), tert-butyl 2,7-diazaspiro[3.5]nonane-2-carboxylate (3.98 g, 17.6 mmol, CAS #236405-55-6), RuPhos (1.64 g, 3.52 mmol), Pd₂(dba)₃ (1.61 g, 1.76 mmol) and t-BuONa (3.38 g, 35.2 mmol) in toluene (60 mL) was degassed and purged with N₂ three times. Then the mixture was stirred at 100° C. for 2 hrs under N₂ atmosphere. On completion, the reaction mixture was quenched with water (30 mL) and extracted with dichloromethane (3×30 mL). The combined organic layers were washed with aqueous NaCl (30 mL) and dried over anhydrous sodium sulfate, filtered and the filtrate was concentrated in vacuo to give the crude residue. The residue was purified by column chromatography (SiO₂, Petroleum ether:Ethyl acetate=19/1) to give the title compound (4.6 g, 70% yield) as a yellow solid. LC-MS (ESI⁺) m, z 373.1 (M+H)⁺.

### Step 2—7-(4-Chloro-2,5-difluorophenyl)-2,7-diazaspiro[3.5]nonane

To a solution of tert-butyl 7-(4-chloro-2,5-difluorophenyl)-2,7-diazaspiro[3.5]nonane-2-carboxylate (4.6 g, 9.9 mmol) in DCM (40 mL) was added TFA (9.82 g, 86.2 mmol). The mixture was then stirred at 25° C. for 1 hr. On completion, the reaction mixture was concentrated under reduced pressure to give the title compound (3 g, TFA) as a black oil. LC-MS (ESI⁺) m/z 273.1 (M+H)⁺.

### 4-(7-(4-Chloro-2,5-difluorophenyl)-2,7-diazaspiro[3.5]nonan-2-yl)-5-fluoro-2-methoxyaniline (Intermediate JO)

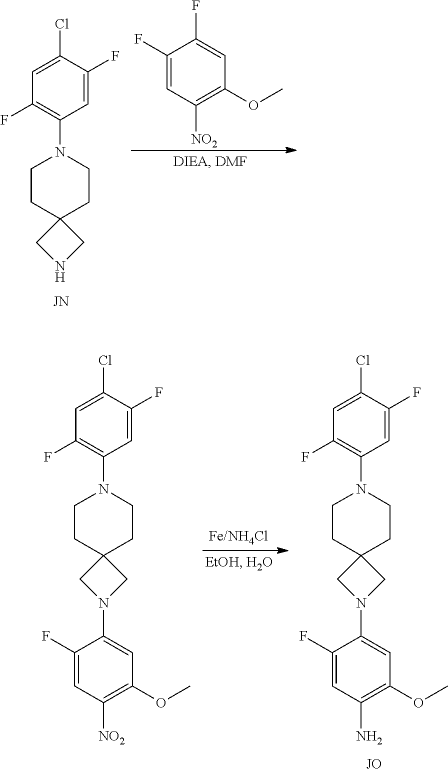

### Step 1—7-(4-Chloro-2,5-difluorophenyl)-2-(2-fluoro-5-methoxy-4-nitrophenyl)-2,7-diazaspiro[3.5]nonane

To a solution of 7-(4-chloro-2,5-difluorophenyl)-2,7-diazaspiro[3.5]nonane (1.5 g. 3.9 mmol, Intermediate JN, TFA), 1,2-difluoro-4-methoxy-5-nitrobenzene (733 mg, 3.88 mmol, CAS #66684-64-8) in DMF (10 mL) was added DIEA (5.01 g, 38.8 mmol). The mixture was then stirred at 80° C. for 4 hrs. On completion, the reaction mixture was quenched with water (30 mL) and extracted with EA (3×30 mL). The combined organic layers were washed with aqueous NaCl (3×30 mL) and dried over anhydrous sodium sulfate, filtered and the filtrate was concentrated in vacuo to give the title compound (2 g) as a yellow solid. LC-MS (ESI⁺) m/z 442.0 (M+H)⁺.

### Step 2—4-(7-(4-Chloro-2,5-difluorophenyl)-2,7-diazaspiro[3.5]nonan-2-yl)-5-fluoro-2-methoxyaniline

A mixture of 7-(4-chloro-2,5-difluorophenyl)-2-(2-fluoro-5-methoxy-4-nitrophenyl)-2,7-diazaspiro[3.5]nonane (800 mg, 1.09 mmol), Fe (607 mg, 10.9 mmol), and NH₄Cl (581 mg, 10.9 mmol) in EtOH (20 mL) and H₂O (10 mL) was degassed and purged with N₂ three times. Then the mixture was stirred at 60° C. for 3 hrs under N₂ atmosphere. On completion, the reaction mixture was quenched with water (30 mL), filtered and filter was extracted by EA (3×30 mL). The combined organic layers were washed by aqueous NaCl (30 mL) and dried over anhydrous sodium sulfate, filtered and the filtrate was concentrated in vacuo to give the title compound (400 mg) as a black solid. LC-MS (ESI⁺) m/z 412.1 (M+H)⁺.

### 3-((4-(7-(4-Chloro-2,5-difluorophenyl)-2,7-diazaspiro[3.5]nonan-2-yl)-5-fluoro-2-methoxyphenyl)amino)piperidine-2,6-dione (Intermediate JP)

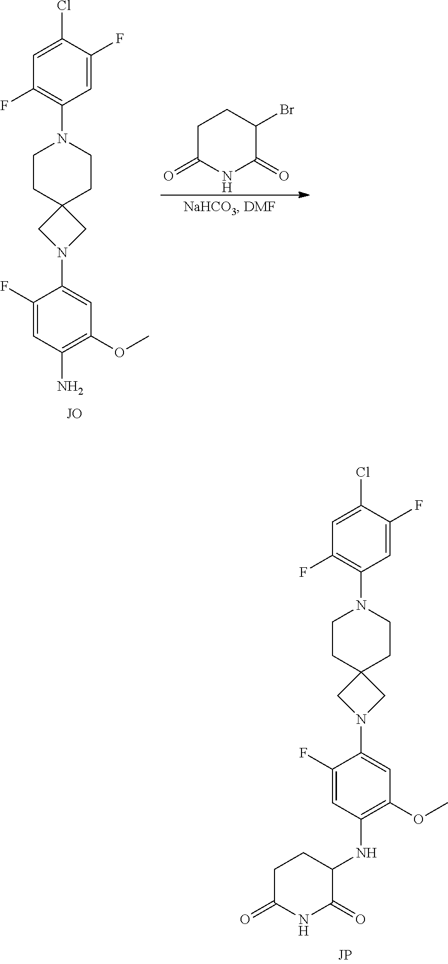 [SMILES: COc1cc(N2CC3(CCN(c4cc(F)c(Cl)cc4F)CC3)C2)c(F)cc1N ; O=C1CCC(Br)C(=O)N1 ; COc1cc(N2CC3(CCN(c4cc(F)c(Cl)cc4F)CC3)C2)c(F)cc1NC1CCC(=O)NC1=O]

To a solution of 4-(7-(4-chloro-2,5-difluorophenyl)-2,7-diazaspiro[3.5]nonan-2-yl)-5-fluoro-2-methoxyaniline (400 mg, 583 μmol, Intermediate JO), and 3-bromopiperidine-2,6-dione (224 mg, 1.17 mmol, CAS #62595-74-8) in DMF (2 mL) was added NaHCO₃ (245 mg, 2.91 mmol). The mixture was then stirred at 80° C. for 24 hrs. On completion, the reaction mixture was quenched with water (30 mL) and extracted with EA (3×30 mL). The combined organic layers were washed with aqueous NaCl (30 mL) and dried over anhydrous sodium sulfate, filtered and the filtrate was concentrated in vacuo to give the crude residue. The residue was purified by column chromatography (SiO₂, Petroleum ether:Ethyl acetate=3/1) to give the title compound (100 mg, 33% yield) as a green solid. LC-MS (ESI⁺) m/z 523.0 (M+H)⁺.

### 2-(4-Chloro-2,5-difluorophenyl)-2,8-diazaspiro[4.5]decane (Intermediate JQ)

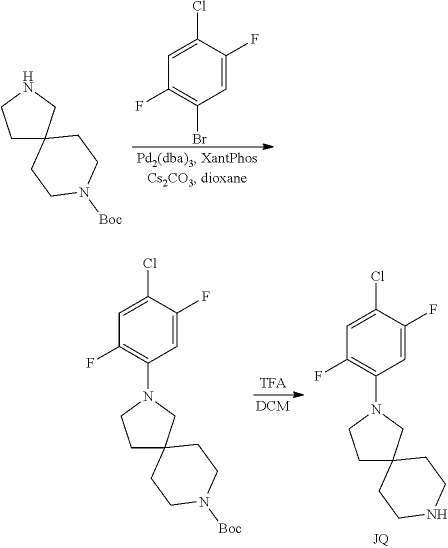 [SMILES: CC(C)(C)OC(=O)N1CCC2(CCNC2)CC1 ; Fc1cc(Br)c(F)cc1Cl ; CC(C)(C)OC(=O)N1CCC2(CC1)CCN(c1cc(F)c(Cl)cc1F)C2 ; Fc1cc(N2CCC3(CCNCC3)C2)c(F)cc1Cl]

### Step 1—Tert-butyl 2-(4-chloro-2,5-difluorophenyl)-2,8-diazaspiro[4.5]decane-8-carboxylate

To a solution of tert-butyl 2,8-diazaspiro[4.5]decane-8-carboxylate (1.6 g, 6.7 mmol, CAS #236406-39-6) in toluene (20 mL) was added 1-bromo-4-chloro-2,5-difluorobenzene (1.51 g, 6.66 mmol, CAS #172921-33-4), tBuONa (1.92 g, 19.9 mmol), Xantphos (385 mg, 665 μmol) and Pd₂(dba)₃ (304 mg, 332 μmol). The reaction was then stirred at 100° C. under N₂ atmosphere for 2 hrs. On completion, the reaction mixture was filtered and the filtrate was concentrated in vacuo to give the crude residue. The residue was purified by column chromatography (SiO₂, Petroleum ether:Ethyl acetate=10/1 to 5/1) to give the title compound (2.3 g, 78% yield) as a brown solid. LC-MS (ESI⁺) m/z 387.1 (M+H)⁺.

### Step 2—2-(4-Chloro-2,5-difluorophenyl)-2,8-diazaspiro[4.5]decane

To a solution of tert-butyl 2-(4-chloro-2,5-difluorophenyl)-2,8-diazaspiro[4.5]decane-8-carboxylate (2.3 g, 6.0 mmol) in DCM (20 mL) was added TFA (7.68 g, 67.3 mmol). The reaction was then stirred at 25° C. for 1 hr. On completion, the reaction mixture was concentrated in vacuo to give the title compound (1.7 g) as a brown oil. LC-MS (ESI⁺) m/z 286.9 (M+H)⁺.

### 4-(2-(4-Chloro-2,5-difluorophenyl)-2,8-diazaspiro[4.5]decan-8-yl)-5-fluoro-2-methoxyaniline (Intermediate JR)

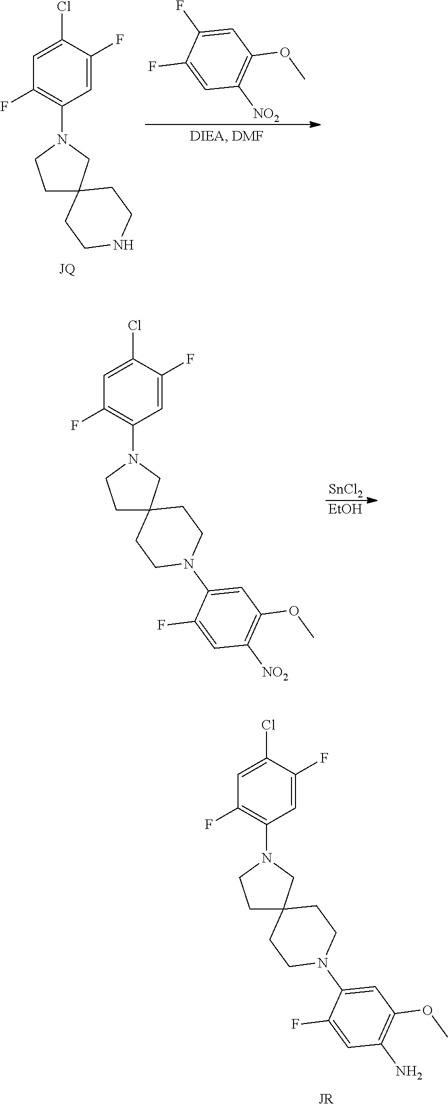

### Step 1—2-(4-Chloro-2,5-difluorophenyl)-8-(2-fluoro-5-methoxy-4-nitrophenyl)-2,8-diazaspiro[4.5]decane

To a solution of 2-(4-chloro-2,5-difluorophenyl)-2,8-diazaspiro[4.5]decane (1.53 g, 5.34 mmol, Intermediate JQ) in DMF (10 mL) was added 1,2-difluoro-4-methoxy-5-nitrobenzene (1.01 g, 5.34 mmol) and DIEA (3.45 g, 26.6 mmol). The reaction was then stirred at 80° C. for 12 hrs. On completion, the reaction mixture was quenched with water (20 mL) and the reaction mixture was filtered and the filter cake was dried in vacuo to give the title compound (2.3 g) as a brown solid. LC-MS (ESI⁺) m/z 455.9 (M+H)

### Step 2—4-(2-(4-Chloro-2,5-difluorophenyl)-2,8-diazaspiro[4.5]decan-8-yl)-5-fluoro-2-methoxyaniline

To a solution of 2-(4-chloro-2,5-difluorophenyl)-8-(2-fluoro-5-methoxy-4-nitrophenyl)-2,8-diazaspiro[4.5]decane (1 g, 2.19 mmol) in EtOH (15 mL) was added dichlorotin (2.08 g, 10.9 mmol). The reaction was then stirred at 80° C. for 2 hrs. On completion, the reaction mixture was quenched with NaHCO₃ until the pH was 7. The reaction mixture was then filtered and the filtrate was concentrated in vacuo to give the title compound (520 mg) as a yellow solid. LC-MS (ESI⁺) m/z 426.1 (M+H)⁺.

### 3-((4-(2-(4-Chloro-2,5-difluorophenyl)-2,8-diazaspiro[4.5]decan-8-yl)-5-fluoro-2-methoxyphenyl)amino)piperidine-2,6-dione (Intermediate JS)

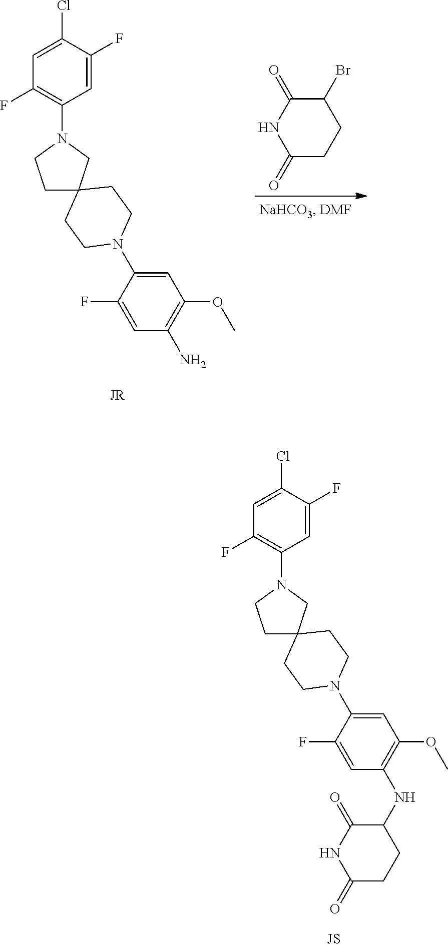 [SMILES: COc1cc(N2CCC3(CC2)CCN(c2cc(F)c(Cl)cc2F)C3)c(F)cc1N ; O=C1CCC(Br)C(=O)N1 ; COc1cc(N2CCC3(CC2)CCN(c2cc(F)c(Cl)cc2F)C3)c(F)cc1NC1CCC(=O)NC1=O]

To a solution of 4-(2-(4-chloro-2,5-difluorophenyl)-2,8-diazaspiro[4.5]decan-8-yl)-5-fluoro-2-methoxyaniline (200 mg, 469 μmol, Intermediate JR) in DMF (4 mL) was added 3-bromopiperidine-2,6-dione (225 mg, 1.17 mmol, CAS #62595-74-8) and NaHCO₃ (78.9 mg, 939 μmol). The reaction was then stirred at 80° C. for 4 hrs. On completion, the reaction mixture was concentrated in vacuo to give the crude residue. The crude residue was purified by reversed-phase HPLC (0.8 g/L ammonium bicarbonate) to give the title compound (160 mg, 56% yield) as a yellow solid. LC-MS (ESI⁺) m/z 535.9 (M+H)⁺.

### 3-(4-Chloro-2,5-difluorophenyl)-3,9-diazaspiro[5.5]undecane (Intermediate JT)

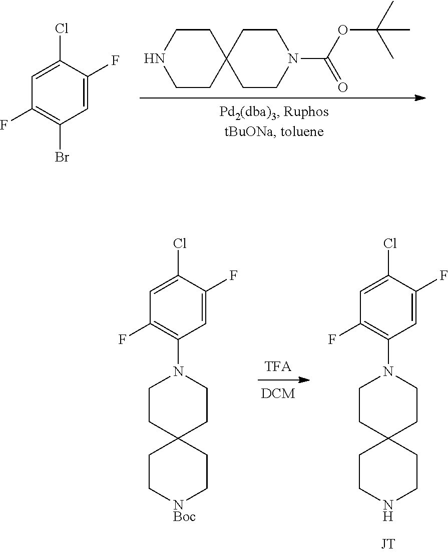

### Step 1—Tert-butyl 9-(4-chloro-2,5-difluorophenyl)-3,9-diazaspiro[5.5]undecane-3-carboxylate

A mixture of tert-butyl 3,9-diazaspiro[5.5]undecane-3-carboxylate (2.24 g, 8.79 mmol, CAS #173405-78-2), 1-bromo-4-chloro-2,5-difluorobenzene (2 g. 9 mmol). RuPhos (821 mg, 1.76 mmol, CAS #172921-33-4), Pd₂(dba)₃ (805 mg, 880 μmol) and t-BuONa (1.69 g, 17.6 mmol) in toluene (30 mL) was degassed and purged with N₂ three times. Then the mixture was stirred at 100° C. for 2 hrs under N₂ atmosphere. On completion, the reaction mixture was quenched with water (30 mL) and extracted with dichloromethane (3×30 mL). The combined organic layers were washed with aqueous NaCl (30 mL) and dried over anhydrous sodium sulfate, filtered and the filtrate was concentrated in vacuo to give the crude residue. The residue was purified by column chromatography (SiO₂, Petroleum ether:Ethyl acetate=5/1). The crude residue was purified by reversed-phase HPLC (0.1% FA condition) to give the title compound (2.5 g, 57% yield) as a yellow oil solid. LC-MS (ESI⁺) m/z 401.1 (M+H)⁺.

### Step 2—3-(4-Chloro-2,5-difluorophenyl)-3,9-diazaspiro[5.5]undecane

A mixture of tert-butyl 9-(4-chloro-2,5-difluorophenyl)-3,9-diazaspiro[5.5]undecane-3-carboxylate (2.5 g, 5.0 mmol), TFA (4.91 g. 43.1 mmol) in DCM (20 mL) was degassed and purged with N₂ three times, and then the mixture was stirred at 25° C. for 1 hr under N₂ atmosphere. On completion, the reaction mixture was concentrated under reduced pressure to give the title compound (2 g, TFA) as a yellow oil. LC-MS (ESI⁺) m/z 301.1 (M+H)⁺.

### 4-(9-(4-Chloro-2,5-difluorophenyl)-3,9-diazaspiro[5.5]undecan-3-yl)-5-fluoro-2-methoxyaniline (Intermediate JU)

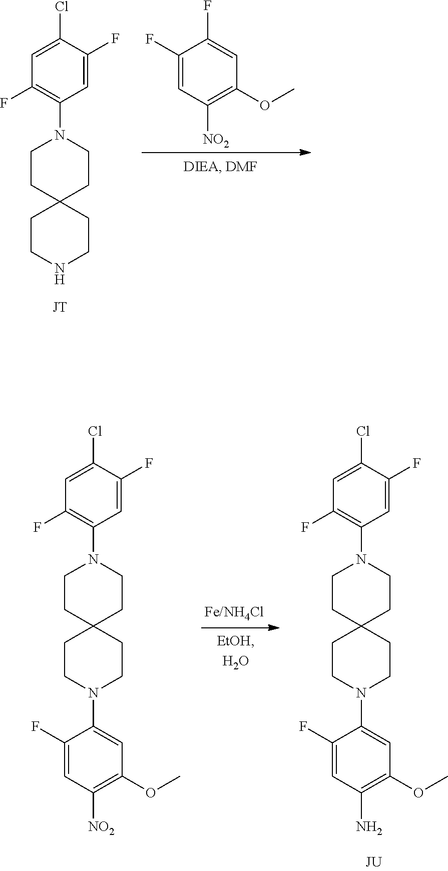

### Step 1—3-(4-Chloro-2,5-difluorophenyl)-9-(2-fluoro-5-methoxy-4-nitrophenyl)-3,9-diazaspiro[5.5]undecane

To a solution of 3-(4-chloro-2,5-difluorophenyl)-3,9-diazaspiro[5.5]undecane (1.5 g, 5.0 mmol, Intermediate JT), 1,2-difluoro-4-methoxy-5-nitrobenzene (943 mg, 4.99 mmol, CAS #66684-64-8) in DMF (15 mL) was added DIEA (6.45 g, 49.9 mmol). The mixture was then stirred at 80° C. for 12 hrs. On completion, the reaction mixture was quenched with water (30 mL), filtered and filter cake was dried in vacuo to give the title compound (2 g) as a yellow solid. LC-MS (ESI⁺) m/z 470.1 (M+H)⁺.

### Step 2—4-(9-(4-Chloro-2,5-difluorophenyl)-3,9-diazaspiro[5.5]undecan-3-yl)-5-fluoro-2-methoxyaniline

A mixture of 3-(4-chloro-2,5-difluorophenyl)-9-(2-fluoro-5-methoxy-4-nitrophenyl)-3,9-diazaspiro[5.5]undecane (500 mg, 1.06 mmol), Fe (594 mg, 10.6 mmol), NH₄Cl (569 mg, 10.6 mmol) in EtOH (8 mL) and H₂O (4 mL) was degassed and purged with N₂ 3 times. Then the mixture was stirred at 60° C. for 2 hrs under N₂ atmosphere. On completion, the reaction mixture was quenched with water (30 mL) and filtered and filter was extracted by EA (3×30 mL). The combined organic layers were washed with aqueous NaCl (30 mL) and dried over anhydrous sodium sulfate, filtered and the filtrate was concentrated in vacuo to give the title compound (400 mg) as a black oil. LC-MS (ESI⁺) m/z 439.8 (M+H)⁺.

### 3-((4-(9-(4-Chloro-2,5-difluorophenyl)-3,9-diazaspiro[5.5]undecan-3-yl)-5-fluoro-2-methoxyphenyl)amino)piperidine-2,6-dione (Intermediate JV)

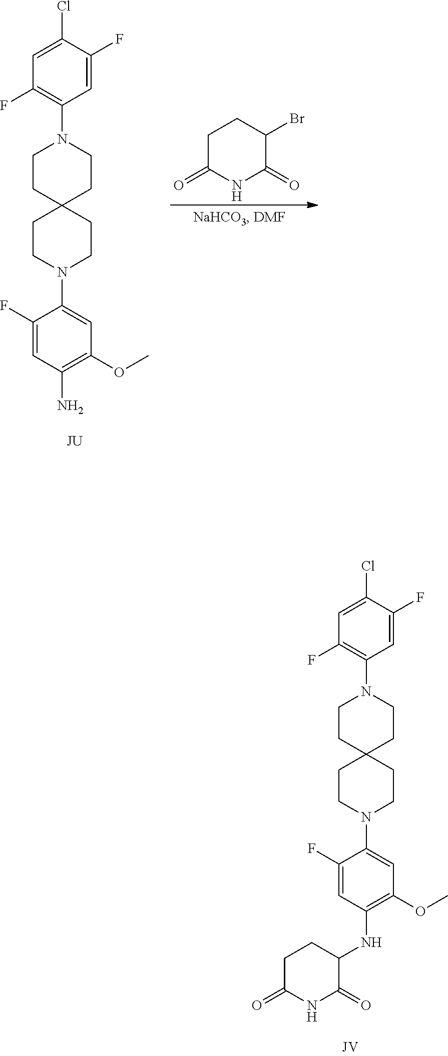 [SMILES: COc1cc(N2CCC3(CCN(c4cc(F)c(Cl)cc4F)CC3)CC2)c(F)cc1N ; O=C1CCC(Br)C(=O)N1 ; COc1cc(N2CCC3(CCN(c4cc(F)c(Cl)cc4F)CC3)CC2)c(F)cc1NC1CCC(=O)NC1=O]

To a solution of 4-(9-(4-chloro-2,5-difluorophenyl)-3,9-diazaspiro[5.5]undecan-3-yl)-5-fluoro-2-methoxyaniline (400 mg, 546 μmol, Intermediate JU), 3-bromopiperidine-2,6-dione (210 mg, 1.09 mmol. CAS #62595-74-8) in DMF (2 mL) was added NaHCO₃ (229 mg, 2.73 mmol). The mixture was then stirred at 80° C. for 24 hrs. On completion, the reaction mixture was quenched with water (30 mL) and extracted with EA (3×30 mL). The combined organic layers were washed with aqueous NaCl (30 mL) and dried over anhydrous sodium sulfate, filtered and the filtrate was concentrated in vacuo to give the crude residue. The residue was purified by column chromatography (SiO₂, Petroleum ether:Ethyl acetate=2/1) to give the title compound (180 mg, 60% yield) as a green solid. LC-MS (ESI⁺) m/z 551.2 (M+H)⁺.

### 1-(4-Chloro-2,5-difluorophenyl)piperazine (Intermediate JW)

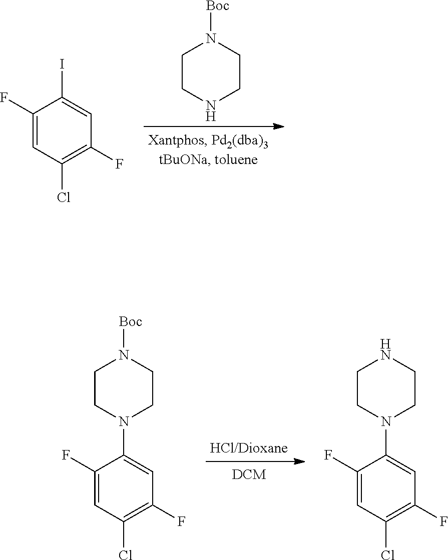 [SMILES: Fc1cc(I)c(F)cc1Cl ; CC(C)(C)OC(=O)N1CCNCC1 ; CC(C)(C)OC(=O)N1CCN(c2cc(F)c(Cl)cc2F)CC1 ; Fc1cc(N2CCNCC2)c(F)cc1Cl]

### Step 1—Tert-butyl 4-(4-chloro-2,5-difluorophenyl)piperazine-1-carboxylate

A mixture of 1-chloro-2,5-difluoro-4-iodobenzene (1 g, 4 mmol, CAS #1097871-23-2), tert-butyl piperazine-1-carboxylate (566 mg, 3.04 mmol, CAS #57260-71-6), tert-butyl piperazine-1-carboxylate (566 mg, 3.04 mmol), and tBuONa (1.46 g, 15.2 mmol) in toluene (20 mL) was degassed and purged with N₂ three times. Then Pd₂(dba)₃ (278 mg, 304 μmol) and Xantphos (176 mg, 307 μmol) was added and the mixture was stirred at 100° C. for 2 hrs under N₂ atmosphere. On completion, the reaction mixture was concentrated under reduced pressure to give a residue. The residue was purified by column chromatography (SiO₂, Petroleum ether:Ethyl acetate=1/0 to 5/1) to give the title compound (990 mg, 88% yield) as a brown solid. LC-MS (ESI⁺) m/z 276.9 (M+H−56)⁺.

### Step 2—1-(4-Chloro-2,5-difluorophenyl)piperazine

To a solution of tert-butyl 4-(4-chloro-2,5-difluorophenyl)piperazine-1-carboxylate (3 g, 9 mmol) in DCM (20 mL) was added HCl/dioxane (2 M, 10 mL). The mixture was then stirred at 25° C. for 12 hrs. On completion, the reaction mixture was concentrated in vacuo to give the crude residue. The crude residue was purified by reversed-phase HPLC (0.8 g/L ammonium bicarbonate) to give the title compound. (1.5 g, 69% yield) as a white solid. LC-MS (ESI⁺) m/z 233.1 (M+H)⁺.

### 4-(4-(4-(4-Chloro-2,5-difluorophenyl)piperazin-1-yl)piperidin-1-yl)-5-fluoro-2-methoxyaniline (Intermediate JX)

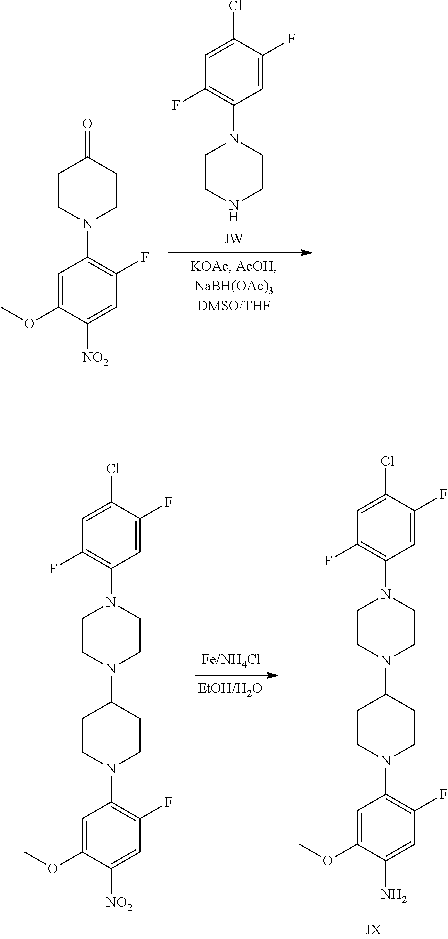 [SMILES: COc1cc(N2CCC(=O)CC2)c(F)cc1[N+](=O)[O-] ; Fc1cc(N2CCNCC2)c(F)cc1Cl ; COc1cc(N2CCC(N3CCN(c4cc(F)c(Cl)cc4F)CC3)CC2)c(F)cc1[N+](=O)[O-] ; COc1cc(N2CCC(N3CCN(c4cc(F)c(Cl)cc4F)CC3)CC2)c(F)cc1N]

### Step 1—1-(4-Chloro-2,5-difluorophenyl)-4-(1-(2-fluoro-5-methoxy-4-nitrophenyl)piperidin-4-yl)piperazine

To a solution of 1-(2-fluoro-5-methoxy-4-nitrophenyl)piperidin-4-one (691 mg, 2.58 mmol, synthesized via Step 1 of Intermediate GI) and 1-(4-chloro-2,5-difluorophenyl)piperazine (600 mg, 2.58 mmol, Intermediate JW) in DMSO (10 mL) and THF (10 mL) was added dropwise AcOH (464 mg, 7.74 mmol) and KOAc (759 mg, 7.74 mmol) at 25° C. After addition, the mixture was stirred at 25° C. for 1 hour, and then NaBH(OAc)₃ (1.64 g, 7.74 mmol) was added. The resulting mixture was stirred at 25° C. for 1 hr. On completion, the reaction mixture was diluted with water (20 mL) and extracted with ethyl acetate (3×50 mL). The combined organic layers were washed with brine (20 mL) and dried over anhydrous sodium sulfate, filtered and the filtrate was concentrated in vacuo to give the title compound (1 g) as a yellow solid. LC-MS (ESI⁺) m/z 484.8 (M+H)⁻.

### Step 2—4-(4-(4-(4-Chloro-2,5-difluorophenyl)piperazin-1-yl)piperidin-1-yl)-5-fluoro-2-methoxyaniline

To a solution of 1-(4-chloro-2,5-difluorophenyl)-4-(1-(2-fluoro-5-methoxy-4-nitrophenyl)piperidin-4-yl)piperazine (500 mg, 1.03 mmol) in EtOH (3 mL), H₂O (3 mL) was added Fe (287 mg, 5.16 mmol) and NH₄Cl (441 mg, 8.25 mmol), then the reaction was stirred at 80° C. for 2 hrs. On completion, the reaction mixture was filtered and the filtrate was concentrated in vacuo to give the crude residue. The crude residue was purified by column chromatography (SiO₂, Petroleum ether/Ethyl acetate=1/0 to 1/1) to give the title compound (400 mg. 73% yield) as a yellow solid. LC-MS (ESI⁺) m/z 454.8 (M+H)⁺.

### 3-((4-(4-(4-(4-Chloro-2,5-difluorophenyl)piperazin-1-yl)piperidin-1-yl)-5-fluoro-2-methoxyphenyl)amino)piperidine-2,6-dione (Intermediate JY)

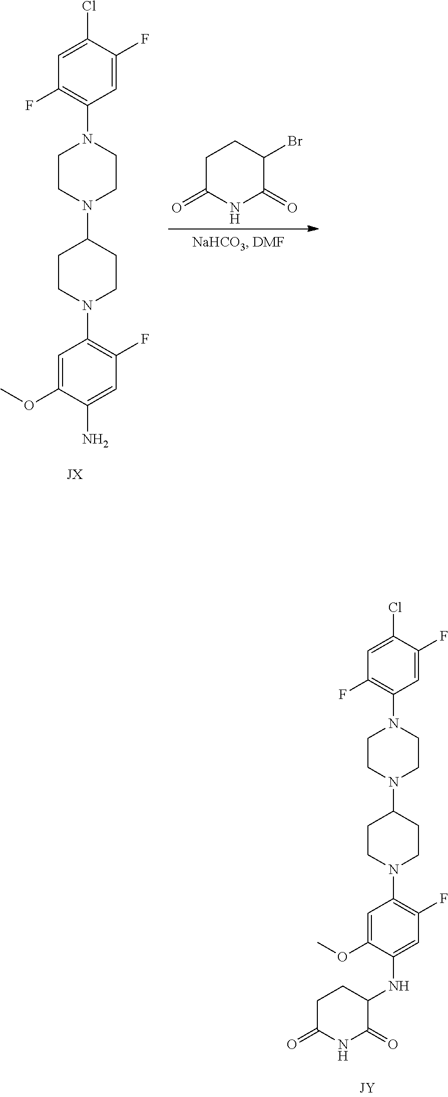 [SMILES: COc1cc(N2CCC(N3CCN(c4cc(F)c(Cl)cc4F)CC3)CC2)c(F)cc1N ; O=C1CCC(Br)C(=O)N1 ; COc1cc(N2CCC(N3CCN(c4cc(F)c(Cl)cc4F)CC3)CC2)c(F)cc1NC1CCC(=O)NC1=O]

To a solution of 4-(4-(4-(4-chloro-2,5-difluorophenyl)piperazin-1-yl)piperidin-1-yl)-5-fluoro-2-methoxyaniline (200 mg, 439 μmol, Intermediate JX), 3-bromopiperidine-2,6-dione (168 mg, 879 μmol, CAS #62595-74-8) in anhydrous DMF (2 mL) was added NaHCO₃ (73.8 mg, 879 mold), then the reaction was stirred at 80° C. for 12 hrs. On completion, the reaction mixture was diluted with water (10 mL) and extracted with ethyl acetate (3×30 mL). The combined organic layers were washed with brine (10 mL), dried over anhydrous sodium sulfate, filtered and the filtrate was concentrated in vacuo to give the crude residue. The crude residue was purified by column chromatography (SiO₂, Petroleum ether/Ethyl acetate=1/0 to 1/1) to give the title compound (150 mg, 58% yield) as a yellow solid. LC-MS (ESI⁺) m/z 566.0 (M+H)⁺.

### 1-(1-(4-Chloro-3-fluorophenyl)piperidin-4-yl)piperazine (Intermediate JZ)

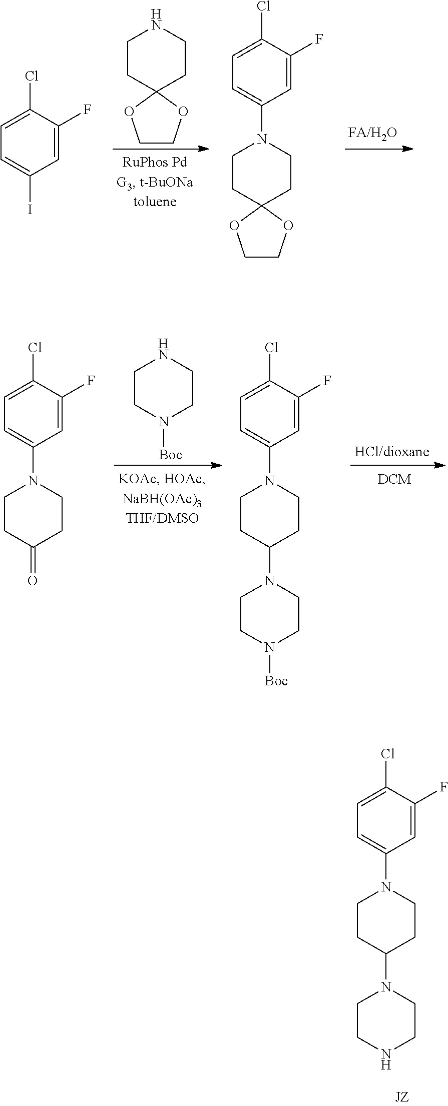

### Step 1—8-(4-Chloro-3-fluorophenyl)-1,4-dioxa-8-azaspiro[4.5]decane

A mixture of 1-chloro-2-fluoro-4-iodo-benzene (6 g, 20 mmol, CAS #202982-67-0) 1,4-dioxa-8-azaspiro[4.5]decane (10.1 g, 70.2 mmol, CAS #177-11-7), t-BuONa (2.70 g, 28.1 mmol), and RuPhos Pd G3 (391 mg, 468 μmol) in toluene (100 mL) was degassed and purged with N₂ three times. Then the mixture was stirred at 100° C. for 4 hrs under N₂ atmosphere. On completion, the reaction mixture was concentrated under reduced pressure to give a residue. The residue was purified by column chromatography (SiO₂, Petroleum ether/Ethyl acetate=1/0 to 5/1) to give the title compound (5.59 g, 84% yield) as a yellow solid. LC-MS (ESI⁺) m/z 272.0 (M+H)⁺; ¹H NMR (400 MHz. DMSO-d₆) δ=7.30 (t, J=9.2 Hz, 1H), 6.96 (dd, J=2.8, 13.2 Hz, 1H), 6.78 (dd, J=2.4, 9.2 Hz, 1H), 3.90 (s, 4H), 3.33-3.30 (m, 4H), 1.71-1.62 (m, 4H).

### Step 2—1-(4-Chloro-3-fluorophenyl)piperidin-4-one

To a solution of 8-(4-chloro-3-fluoro-phenyl)-1,4-dioxa-8-azaspiro[4.5]decane (5 g, 20 mmol) in H₂O (15 mL) was added HCOOH (15 mL). The mixture was then stirred at 80° C. for 5 hrs. On completion, the reaction mixture was concentrated under reduced pressure to remove most of FA and then 3M NaOH was added to adjust the pH to 7. The mixture was then concentrated under reduced pressure to give a residue. The residue was purified by column chromatography (SiO₂, Petroleum ether/Ethyl acetate=1/0 to 20/1) to give the title compound (3.28 g, 76% yield) as a yellow solid. LC-MS (ESI⁺) m/z 228.0 (M+H)⁺; ¹H NMR (400 MHz, DMSO-d₆) δ=7.34 (t, J=8.8 Hz, 1H), 7.04 (dd, J=2.8, 13.2 Hz, 1H), 6.84 (dd, J=2.4, 9.2 Hz, 1H), 3.63 (t, J=6.0 Hz, 4H), 2.40 (t, J=6.0 Hz, 4H).

### Step 3—Tert-butyl 4-(1-(4-chloro-3-fluorophenyl)piperidin-4-yl)piperazine-1-carboxylate

To a solution of 1-(4-chloro-3-fluoro-phenyl)piperidin-4-one (1 g, 4 mmol) and tert-butyl piperazine-1-carboxylate (1.23 g, 6.59 mmol, CAS #5 7260-71-6) in THF (16 mL) and DMSO (4 mL) was added KOAc (1.29 g, 13.2 mmol) and HOAc (1.32 g, 22.0 mmol). The mixture was then stirred at 25° C. for 12 hrs. Then, NaBH(OAc)₃ (2.79 g, 13.2 mmol) was added and the mixture was stirred at 25° C. for 1 hr. On completion, the reaction mixture was quenched by addition of H₂O (20 mL) at 0° C., and then extracted with EA (20 mL×3). The combined organic layers were washed with brine (20 mL×3), dried over Na₂SO₄, filtered and concentrated under reduced pressure to give a residue. The residue was purified by column chromatography (SiO₂, Petroleum ether/Ethyl acetate=3/1 to 0/1) to give the title compound (690 mg, 36% yield) as a yellow solid. LC-MS (ESI⁺) m/z 398.2 (M+H)⁺.

### Step 4—1-(1-(4-Chloro-3-fluorophenyl)piperidin-4-yl)piperazine

To a solution of tert-butyl 4-[1-(4-chloro-3-fluoro-phenyl)-4-piperidyl]piperazine-1-carboxylate (690 mg, 1.73 mmol) in DCM (20 mL) was added HCl/dioxane (8 M, 3 mL). The mixture was stirred at 25° C. for 2 hrs. On completion, the reaction mixture was concentrated under reduced pressure to give the title compound (800 mg, HCl) as a yellow solid. LC-MS (ESI⁺) m/z 298.1 (M+H)⁺.

### 4-(4-(1-(4-Chloro-3-fluorophenyl)piperidin-4-yl)piperazin-1-yl)-5-fluoro-2-methoxyaniline (Intermediate KA)

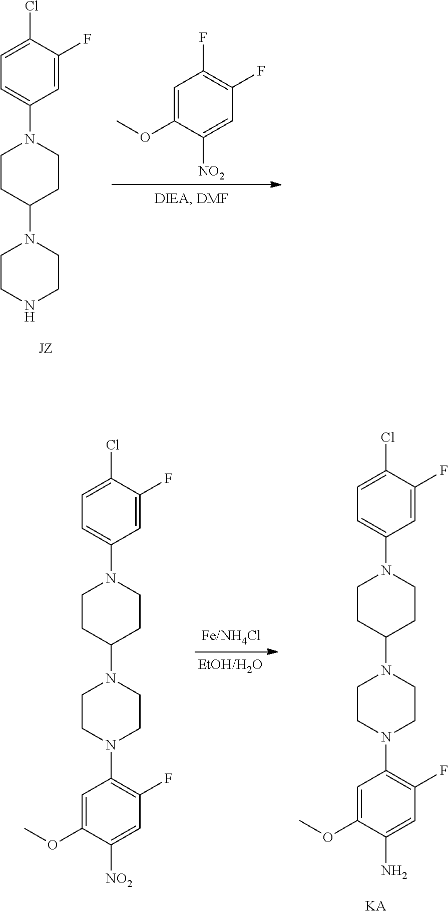 [SMILES: Fc1cc(N2CCC(N3CCNCC3)CC2)ccc1Cl ; COc1cc(F)c(F)cc1[N+](=O)[O-] ; COc1cc(N2CCN(C3CCN(c4ccc(Cl)c(F)c4)CC3)CC2)c(F)cc1[N+](=O)[O-] ; COc1cc(N2CCN(C3CCN(c4ccc(Cl)c(F)c4)CC3)CC2)c(F)cc1N ; "KA"]

### Step 1—1-(1-(4-Chloro-3-fluorophenyl)piperidin-4-yl)-4-(2-fluoro-5-methoxy-4-nitrophenyl)piperazine

To a solution of 1-[1-(4-chloro-3-fluoro-phenyl)-4-piperidyl]piperazine (580 mg, 1.74 mmol. HCl, Intermediate JZ) and 1,2-difluoro-4-methoxy-5-nitro-benzene (328 mg, 1.74 mmol, CAS #66684-64-8) in DMF (10 mL) was added K₂CO₃ (719 mg, 5.21 mmol) and DIEA (673 mg, 5.21 mmol). The mixture was then stirred at 90° C. for 3 hrs. On completion, the reaction mixture was poured into H₂O (80 mL) and stirred for 0.5 hrs. The crude product was triturated with H₂O at 25° C. for 0.5 hrs and then filtered to give the filter cake as the title compound (570 mg, 57% yield) as a yellow solid. LC-MS (ESI⁺) m/z 466.9 (M+H)⁺.

### Step 2—4-(4-(1-(4-Chloro-3-fluorophenyl)piperidin-4-yl)piperazin-1-yl)-5-fluoro-2-methoxyaniline

To a solution of 1-[1-(4-chloro-3-fluoro-phenyl)-4-piperidyl]-4-(2-fluoro-5-methoxy-4-nitro-phenyl)piperazine (300 mg, 643 μmol) in EtOH (5 mL) and H₂O (5 mL) was added Fe (252 mg, 4.50 mmol) and NH₄Cl (241 mg, 4.50 mmol). The mixture was stirred at 80° C. for 2 hrs. On completion, the reaction mixture was filtered to give the filtrate. The filtrate was diluted with H₂O (20 mL), added NH3·H₂O to adjust the pH to 8 and extracted with EA (30 mL×3). The combined organic layers were dried over Na₂SO₄, filtered and concentrated under reduced pressure to give the title compound (240 mg) as a yellow solid. LC-MS (ESI+) m/z 437.2 (M+H)⁺; ¹H NMR (400 MHz, DMSO-d₆) δ=7.29 (t, J=8.9 Hz, 1H), 6.94 (dd, J=2.5, 13.4 Hz, 1H), 6.77 (dd, J=2.0, 9.2 Hz, 1H), 6.53 (d, J=8.0 Hz, 1H), 6.42 (d, J=13.6 Hz, 1H), 3.79-3.70 (m, 4H), 3.30-3.27 (m, 1H), 2.87 (s, 3H), 2.73 (t, J=11.6 Hz, 2H), 2.68-2.56 (m, 4H), 2.45-2.35 (m, 1H), 1.84 (J=12.0 Hz, 2H), 1.54-1.42 (m, 2H).

### 3-((4-(4-(1-(4-Chloro-3-fluorophenyl)piperidin-4-yl)piperazin-1-yl)-5-fluoro-2-methoxyphenyl)amino)piperidine-2,6-dione (Intermediate KB)

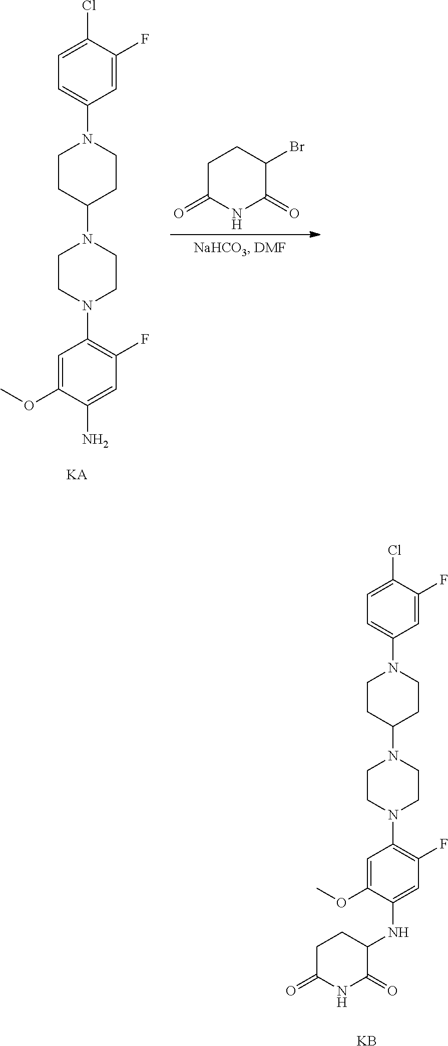 [SMILES: COc1cc(N2CCN(C3CCN(c4ccc(Cl)c(F)c4)CC3)CC2)c(F)cc1N ; O=C1CCC(Br)C(=O)N1 ; "KA" ; COc1cc(N2CCN(C3CCN(c4ccc(Cl)c(F)c4)CC3)CC2)c(F)cc1NC1CCC(=O)NC1=O ; "KB"]

To a solution of 4-[4-[1-(4-chloro-3-fluoro-phenyl)-4-piperidyl]piperazin-1-yl]-5-fluoro-2-methoxy-aniline (240 mg, 549 μmol, Intermediate KA) in DMF (8 mL) was added NaHCO₃ (138 mg, 1.65 mmol) and 3-bromopiperidine-2,6-dione (316 mg, 1.65 mmol). The mixture was then stirred at 80° C. for 12 hrs. On completion, the reaction mixture was diluted with H₂O (30 mL) and extracted with EA (50 mL×3). The combined organic layers were washed with brine (30 mL×3), dried over Na₂SO₄, filtered and concentrated under reduced pressure to give a residue. The residue was purified by column chromatography (SiO₂, DCM/EA=1/0 to 0/1) to give the title compound (73 mg. 20% yield) as a yellow solid. LC-MS (ESI⁺) m/z 548.2 (M+H)⁺.

### (S)-6-(1-(3-(1H-pyrazol-1-yl)propanoyl)piperidin-3-yl)-7-fluoro-N,N-dimethyl-4-(4,4,5,5-tetramethyl-1,3,2-dioxaborolan-2-yl)-1H-indole-2-carboxamide (Intermediate KC) & (R)-6-(1-(3-(1H-pyrazol-1-yl)propanoyl)piperidin-3-yl)-7-fluoro-N,N-dimethyl-4-(4,4,5,5-tetramethyl-1,3,2-dioxaborolan-2-yl)-1H-indole-2-carboxamide (Intermediate KD)

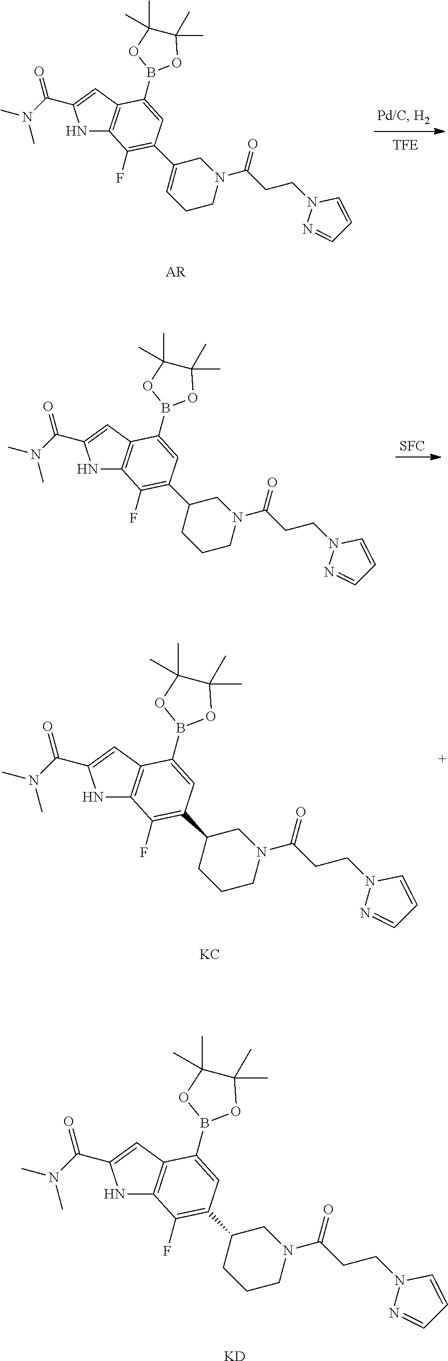

### Step 1—6-(1-(3-(1H-pyrazol-1-yl)propanoyl)piperidin-3-yl)-7-fluoro-N,N-dimethyl-4-(4,4,5,5-tetramethyl-1,3,2-dioxaborolan-2-yl)-1H-indole-2-carboxamide

To a solution of 6-(1-(3-(1H-pyrazol-1-yl)propanoyl)-1,2,5,6-tetrahydropyridin-3-yl)-7-fluoro-N,N-dimethyl-4-(4,4,5,5-tetramethyl-1,3,2-dioxaborolan-2-yl)-1H-indole-2-carboxamide (1 g, 2 mmol, Intermediate AR) in TFE (10 mL) was added Pd/C (1.00 g, 940 μmol, 10 wt %) at 25° C., then the mixture was stirred at 25° C. for 12 hrs. On completion, the reaction mixture was filtered with THF on kieselguhr slowly, keep the system wet, then the filter liquor was concentrated in vacuo to get the crude residue. The residue was purified by column chromatography (SiO₂, Petroleum ether/Ethyl acetate=5/1 to DCM:MeOH=20:1) to give the title compound (700 mg, 66% yield) as a yellow solid. LC-MS (ESI⁺) m/z 538.1 (M+H)⁺.

### Step 2—(S)-6-(1-(3-(1H-pyrazol-1-yl)propanoyl)piperidin-3-yl)-7-fluoro-N,N-dimethyl-4-(4,4,5,5-tetramethyl-1,3,2-dioxaborolan-2-yl)-1H-indole-2-carboxamide & (R)-6-(1-(3-(1H-pyrazol-1-yl)propanoyl)piperidin-3-yl)-7-fluoro-N,N-dimethyl-4-(4,4,5,5-tetramethyl-1,3,2-dioxaborolan-2-yl)-1H-indole-2-carboxamide

The 6-(1-(3-(1H-pyrazol-1-yl)propanoyl)piperidin-3-yl)-7-fluoro-N,N-dimethyl-4-(4,4,5,5-tetramethyl-1,3,2-dioxaborolan-2-yl)-1H-indole-2-carboxamide (700 mg, 1.30 mmol) was separated by SFC (column: REGIS (R,R)WHELK-01 (250 mm*25 mm, 10 um): mobile phase: [CO₂-ACN/i-PrOH (0.1% NH₃H₂O)]; B %:45%, isocratic elution mode) to give the first eluting isomer (S)-6-(1-(3-(1H-pyrazol-1-yl)propanoyl)piperidin-3-yl)-7-fluoro-N,N-dimethyl-4-(4,4,5,5-tetramethyl-1,3,2-dioxaborolan-2-yl)-1H-indole-2-carboxamide (400 mg) as a yellow solid and the second eluting isomer (R)-6-(1-(3-(1H-pyrazol-1-yl)propanoyl)piperidin-3-yl)-7-fluoro-N,N-dimethyl-4-(4,4,5,5-tetrarnethyl-1,3,2-dioxaborolan-2-yl)-1H-indole-2-carboxamide (370 mg) as a yellow solid. LC-MS (ESI⁺) m/z 538.2 (M+H) for both isomers. The absolute stereochemistry of the enantiomers was assigned arbitrarily.

### (S)-6-(1-(3-(1H-1,2,3-triazol-1-yl)propanoyl)piperidin-3-yl)-7-fluoro-N,N-dimethyl-4-(4,4,5,5-tetramethyl-1,3,2-dioxaborolan-2-yl)-TH-indole-2-carboxamide (Intermediate KE) and (R)-6-(1-(3-(1H-1,2,3-triazol-1-yl)propanoyl)piperidin-3-yl)-7-fluoro-N,N-dimethyl-4-(4,4,5,5-tetramethyl-1,3,2-dioxaborolan-2-yl)-1H-indole-2-carboxamide (Intermediate KF)

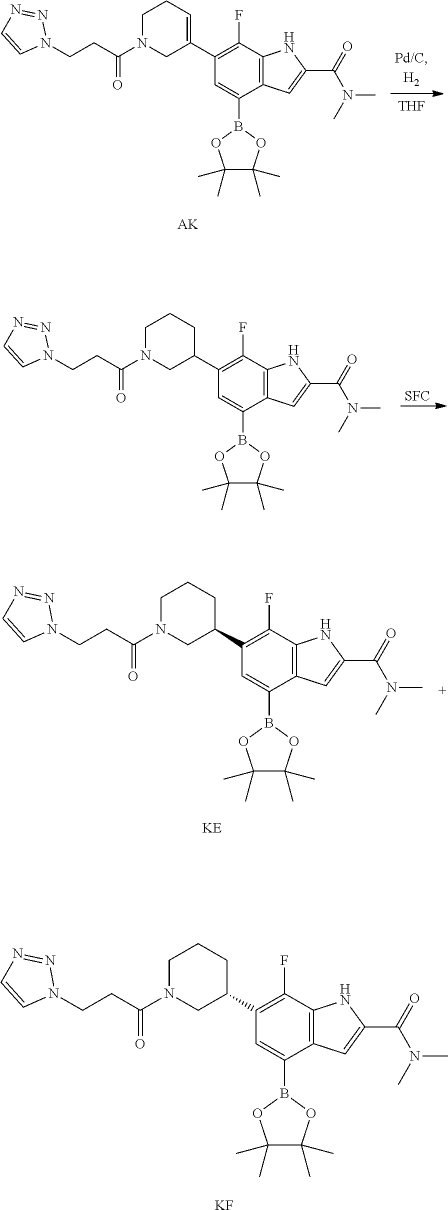

### Step 1—6-(1-(3-(1H-1,2,3-triazol-1-yl)propanoyl)piperidin-3-yl)-7-fluoro-N,N-dimethyl-4-(4,4,5,5-tetramethyl-1,3,2-dioxaborolan-2-yl)-1H-indole-2-carboxamide

To a solution of 7-fluoro-N,N-dimethyl-4-(4,4,5,5-tetramethyl-1,3,2-dioxaborolan-2-yl)-6-[1-[3-(triazol-1-yl)propanoyl]-3,6-dihydro-2H-pyridin-5-yl]-1H-indole-2-carboxamide (1.2 g, 2.2 mmol, Intermediate AK) in THF (20 mL) was added Pd/C (1.2 g, 10 wt %) under N₂ atmosphere. The suspension was degassed and purged with H₂ three times. Then the mixture was stirred under H₂ (15 Psi) at 25° C. for 12 hrs. On completion, the reaction mixture was concentrated under reduced pressure to remove solvent to give the title compound (210 mg, 17% yield) as a white solid. LC-MS (ESI⁺) m/z 457.1 (M+H)⁺.

### Step 2—(S)-6-(1-(3-(1H-1,2,3-triazol-1-yl)propanoyl)piperidin-3-yl)-7-fluoro-N,N-dimethyl-4-(4,4,5,5-tetramethyl-1,3,2-dioxaborolan-2-yl)-1H-indole-2-carboxamide and (R)-6-(1-(3-(1H-1,2,3-triazol-1-yl)propanoyl)piperidin-3-yl)-7-fluoro-N,N-dimethyl-4-(4,4,5,5-tetramethyl-1,3,2-dioxaborolan-2-yl)-1H-indole-2-carboxamide

7-Fluoro-N,N-dimethyl-4-(4,4,5,5-tetramethyl-1,3,2-dioxaborolan-2-yl)-6-[1-[3-(triazol-1-yl)propanoyl]-3-piperidyl]-1H-indole-2-carboxamide (210 mg, 390 μmol) was separated by SFC (column: (s,s) WHELK-01 (250 mm*30 mm, 10 um); mobile phase: [C02-ACN/MeOH (0.1% NH3H2O)]; B %:45%, isocratic elution mode) to give the first eluting isomer (S)-6-(1-(3-(1H-1,2,3-triazol-1-yl)propanoyl)piperidin-3-yl)-7-fluoro-N,N-dimethyl-4-(4,4,5,5-tetramethyl-1,3,2-dioxaborolan-2-yl)-1H-indole-2-carboxamide (60 mg, 28% yield) as a white solid and the second eluting isomer (R)-6-(1-(3-(1H-1,2,3-triazol-1-yl)propanoyl)piperidin-3-yl)-7-fluoro-N,N-dimethyl-4-(4,4,5,5-tetramethyl-1,3,2-dioxaborolan-2-yl)-1H-indole-2-carboxamide (52 mg, 24% yield) as a white solid. LC-MS (ESI⁺) m/z 537.2 (M+H)⁺ for both isomers. The absolute stereochemistry of the enantiomers was assigned arbitrarily.

### (R)-4-(4-(4-chloro-3-methylphenyl)-3,3-difluoropiperidin-1-yl)-5-fluoro-2-methoxyaniline (Intermediate KG)

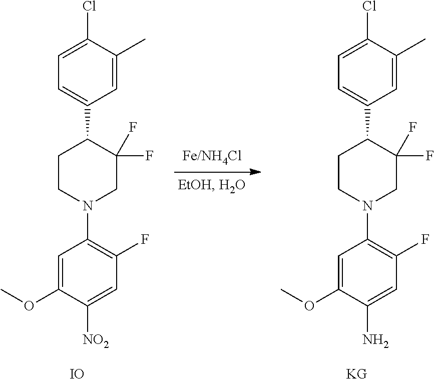

To a solution of (R)-4-(4-chloro-3-methylphenyl)-3,3-difluoro-1-(2-fluoro-5-methoxy-4-nitrophenyl)piperidine (200 mg, 482 μmol, Intermediate IO) in EtOH (2 mL) and H₂O (0.5 mL) was added Fe (135 mg, 2.41 mmol) and NH₄Cl (206 mg, 3.86 mmol). The mixture was then stirred at 80° C. for 2 hrs under N₂ atmosphere. On completion, the reaction mixture was filtered and the filtrate was concentrated in vacuo to give the title compound (130 mg) as a brown solid. LC-MS (ESI⁺) m/z 385.1 (M+H)⁺.

### 3-((4-((R)-4-(4-chloro-3-methylphenyl)-3,3-difluoropiperidin-1-yl)-5-fluoro-2-methoxyphenyl)amino)piperidine-2,6-dione (Intermediate KH)

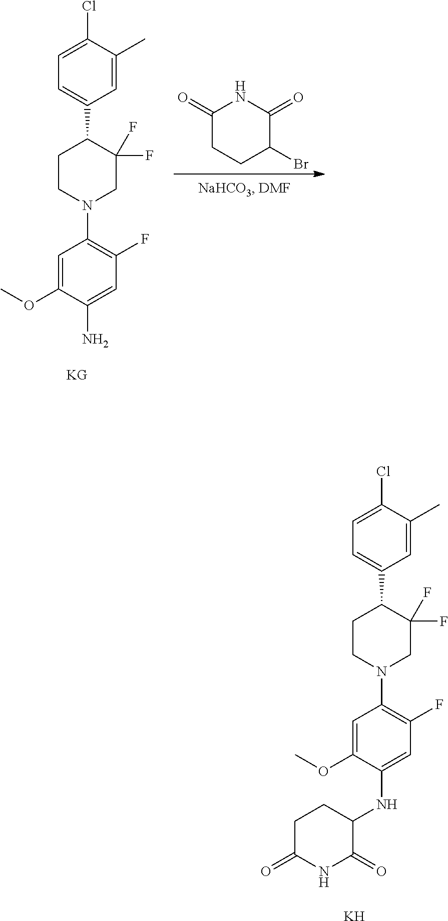

To a solution of (R)-4-(4-(4-chloro-3-methylphenyl)-3,3-difluoropiperidin-1-yl)-5-fluoro-2-methoxyaniline (100 mg, 260 μmol, Intermediate KG) in DMF (2 mL) was added NaHCO₃ (65.5 mg, 780 μmol) and 3-bromopiperidine-2,6-dione (99.79 mg, 520 μmol). The mixture was stirred at 80° C. for 2 hrs. On completion, the reaction mixture was filtered and the filtrate was concentrated in vacuo to give the title compound (110 mg) as a green solid. LC-MS (ESI⁺) m/z 496.1 (M+H)⁺.

### (R)-4-(4-(3,3-difluoropiperidin-4-yl)-2-fluorophenyl)-7-fluoro-6-(1-isobutyryl-1,2,5,6-tetrahydropyridin-3-yl)-N,N-dimethyl-1H-indole-2-carboxamide (Intermediate KI)

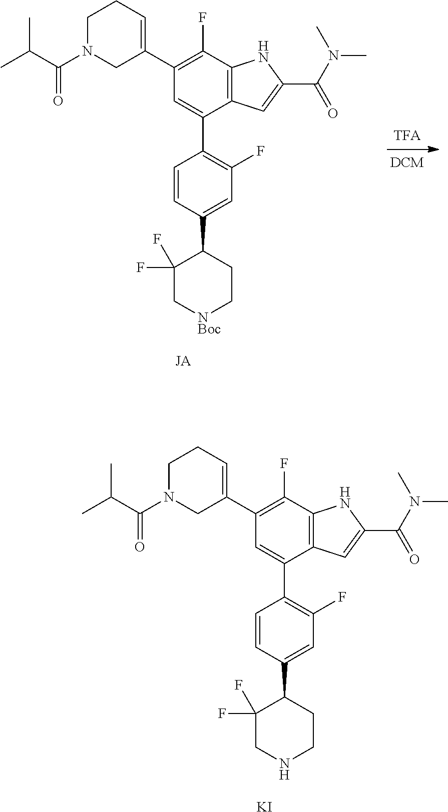

To a solution of tert-butyl (R)-4-(4-(2-(dimethylcarbamoyl)-7-fluoro-6-(1-isobutyryl-1,2,5,6-tetrahydropyridin-3-yl)-1H-indol-4-yl)-3-fluorophenyl)-3,3-difluoropiperidine-1-carboxylate (300 mg, 447 gmol, Intermediate JA) in DCM (3 mL) was added TFA (255 mg, 2.2 mmol, 166 μL) and the mixture was stirred at 25° C. for 1 hr. On completion, the mixture was purified by prep-HPLC (FA condition) to give the title compound (190 mg, 75% yield, FA) as a white solid. LC-MS (ESI⁺) m/z 571.3 (M+H)⁺.

### 2-Chloro-5-fluoro-4-(piperidin-4-yl)phenol Intermediate KJ)

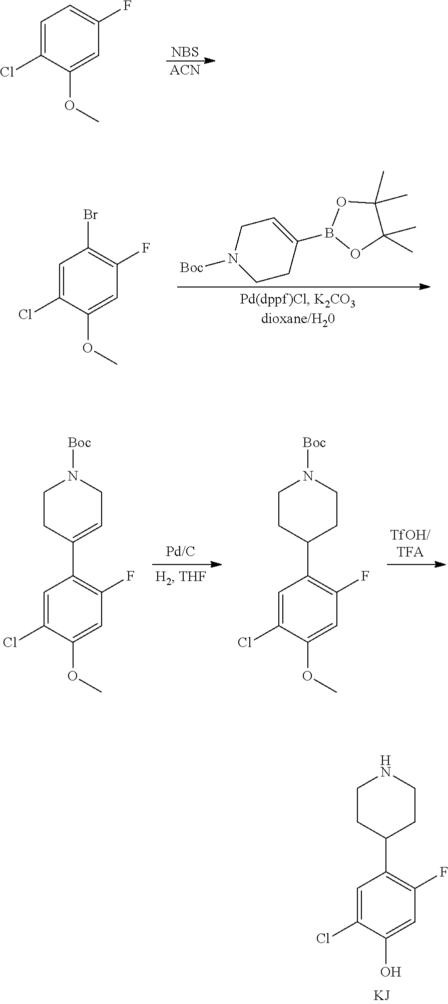

### Step 1—1-Bromo-5-chloro-2-fluoro-4-methoxybenzene

To a solution of 1-chloro-4-fluoro-2-methoxy-benzene (8 g, 50 mmol, CAS #450-89-5) in ACN (100 mL) was added NBS (12.4 g, 69.8 mmol). The mixture was then stirred at 70° C. for 24 hrs. On completion, the reaction mixture was diluted with H₂O (100 mL) and extracted with EA (100 mL×3). The combined organic layers were dried over Na₂SO₄, filtered and concentrated under reduced pressure to give a residue. The residue was purified by column chromatography (SiO₂, Petroleum ether/Ethyl acetate=1/0 to 20/1) to give the title compound (7 g, 54% yield) as a white solid. ¹H NMR (400 MHz, DMSO-d₆) δ=7.83 (d, J=7.2 Hz, 1H), 7.46 (dd, J=6.0, 8.8 Hz, 1H), 7.33 (d, J=10.8 Hz, 1H), 7.14-7.07 (m, 1H), 3.90-3.86 (m, 3H).

### Step 2—Tert-butyl 4-(5-chloro-2-fluoro-4-methoxyphenyl)-5,6-dihydropyridine-1 (2H)-carboxylate

A mixture of 1-bromo-5-chloro-2-fluoro-4-methoxy-benzene (3 g, 12.5 mmol, CAS #901236-75-7), tert-butyl 4-(4,4,5,5-tetramethyl-1,3,2-dioxaborolan-2-yl)-3,6-dihydro-2H-pyridine-1-carboxylate (4.26 g, 13.8 mmol, CAS #286961-14-6), K₂CO₃ (5.19 g, 37.6 mmol), and Pd(dppf)Cl₂ (917 mg, 1.25 mmol) in dioxane (30 mL) and H₂O (8 mL) was degassed and purged with N₂ three times. Then the mixture was stirred at 80° C. for 2 hrs under N₂ atmosphere. On completion, the reaction mixture was concentrated under reduced pressure to give a residue. The residue was purified by column chromatography (SiO₂, Petroleum ether/Ethyl acetate=1/0 to 10/1) to give the title compound (3.44 g, 78% yield) as a yellow solid. LC-MS (ESI⁺) mi/z 286.0 (M−55)⁺; ¹H NMR (400 MHz, DMSO-d₆) δ=7.40 (d, J=8.0 Hz, 1H), 7.10 (d, J=12.8 Hz, 1H), 5.96 (s, 1H), 3.97 (s, 2H), 3.86 (s, 3H), 3.50 (t, J=5.6 Hz, 2H), 2.39 (s, 2H), 1.42 (s, 9H).

### Step 3—Tert-butyl 4-(5-chloro-2-fluoro-4-methoxyphenyl)piperidine-1-carboxylate

To a solution of tert-butyl 4-(5-chloro-2-fluoro-4-methoxy-phenyl)-3,6-dihydro-2H-pyridine-1-carboxylate (2 g, 6 mmol) in THF (20 mL) was added Pd/C (2.00 g, 1.88 mmol, 10 wt %) under Ar. The suspension was degassed under vacuum and purged with H₂ several times. The mixture was then stirred under H₂ (15 psi) at 25° C. for 2 hrs. On completion, the reaction was filtered through kieselguhr carefully, and the filtrate was concentrated in vacuo to give the title compound (1.95 g) as a light-yellow solid. LC-MS (ESI⁺) m/z 288.1 (M−55)⁺.

### Step 4—2-Chloro-5-fluoro-4-(piperidin-4-yl)phenol

A solution of tert-butyl 4-(5-chloro-2-fluoro-4-methoxy-phenyl)piperidine-1-carboxylate (800 mg, 2.33 mmol) in TFA (4 mL) and TfOH (4 mL) was stirred at 80° C. for 1 hr On completion, a stream of N₂ gas was blown onto the reaction mixture to remove most of the acid. Then the pH of the mixture was was adjusted to 6 by 2M NaOH. The residue was purified by prep-HPLC (0.1% FA condition) to give the title compound (530 mg, 81% yield, FA) as a white solid. LC-MS (ESI⁺) mi/z 230.1 (M+H)⁺.

### 2-Chloro-4-(1-(2-(1-(2,6-dioxopiperidin-3-yl)-3-methyl-1H-indazol-4-yl)ethyl)piperidin-4-yl)-5-fluorophenyl trifluoromethanesulfonate (Intermediate KK)

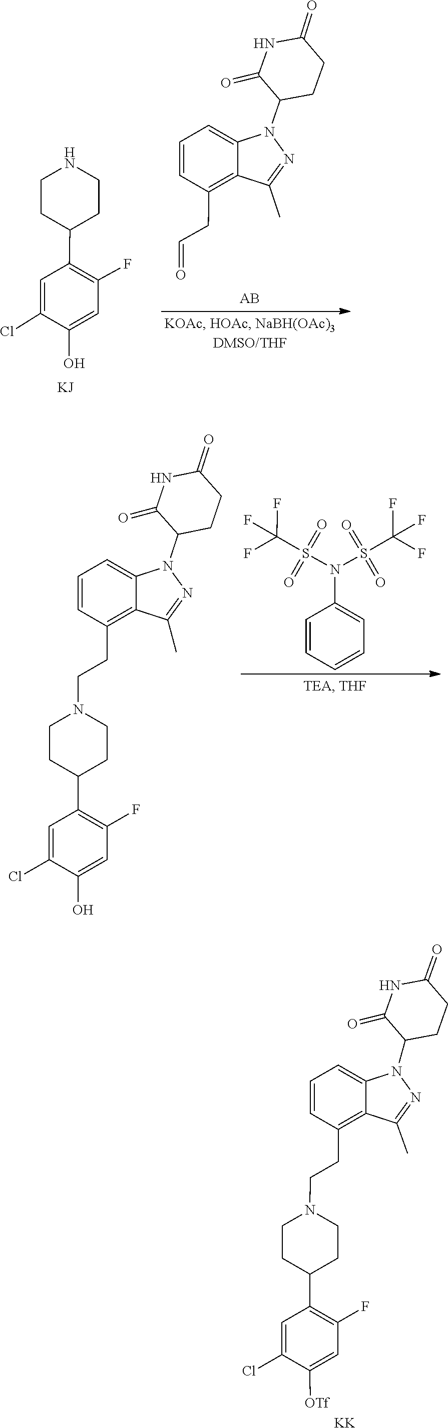

### Step 1—3-(4-(2-(4-(5-Chloro-2-fluoro-4-hydroxyphenyl)piperidin-1-yl)ethyl)-3-methyl-1H-indazol-1-yl)piperidine-2,6-dione

To a solution of 2-[1-(2,6-dioxo-3-piperidyl)-3-methyl-indazol-4-yl]acetaldehyde (220 mg, 771 μmol, Intermediate AB) and 2-chloro-5-fluoro-4-(4-piperidyl)phenol (213 mg, 925 μmol, Intermediate KJ) in THF (4 mL) and DMSO (1 mL) was added KOAc (227 mg, 2.31 mmol), and HOAc (232 mg, 3.86 mmol) at 25° C. After addition, the mixture was stirred at this temperature for 1 hr, and then NaBH(OAc)₃ (490 mg, 2.31 mmol) was added at 0° C. The resulting mixture was stirred at 0° C. for 1 hr. On completion, the reaction mixture was quenched by addition of H₂O (10 mL), and then extracted with EA (15 mL×3). The combined organic layers were dried over Na₂SO₄, filtered and concentrated under reduced pressure to give a residue. The residue was purified by column chromatography (SiO₂, Petroleum ether/Ethyl acetate=l/1 to 0/1 to DCM/MeOH=1/0 to 10/1) to give the title compound (400 mg, 83% yield) as a yellow oil. LC-MS (ESI⁺) m/z 499.2 (M+H)⁺.

### Step 2—2-Chloro-4-(1-(2-(1-(2,6-dioxopiperidin-3-yl)-3-methyl-1H-indazol-4-yl)ethyl)piperidin-4-yl)-5-fluorophenyl trifluoromethanesulfonate

To a solution of 3-[4-[2-[4-(5-chloro-2-fluoro-4-hydroxy-phenyl)-1-piperidyl]ethyl]-3-methyl-indazol-1-yl]piperidine-2,6-dione (360 mg, 721 μmol) in THF (10 mL) was added TEA (219 mg, 2.16 mmol), DMAP (8.81 mg, 72.2 μmol) and then 1,1,1-trifluoro-N-phenyl-N-(trifluoromethylsulfonyl)methanesulfonamide (387 mg, 1.08 mmol, CAS #37595-74-7). The mixture was then stirred at 25° C. for 2 hrs. On completion, the reaction mixture was concentrated under reduced pressure to give a residue. The residue was purified by prep-HPLC (0.1% FA condition) to give the title compound (150 mg, 33% yield) as a white solid. LC-MS (ESI⁺) m/z 631.2 (M+H); ¹H NMR (400 MHz, DMSO-d₆) δ=11.05 (s, 1H), 7.81 (d, J=7.2 Hz, 1H), 7.73 (d, J=9.6 Hz, 1H), 7.40-7.33 (m, 1H), 7.27 (t, J=7.6 Hz, 1H), 6.94 (d, J=6.8 Hz, 1H), 5.72 (dd, J=4.8, 11.6 Hz, 1H), 0.21-3.07 (m, 5H), 2.90-2.79 (m, 2H), 2.76-2.68 (m, 2H), 2.66-2.59 (m, 5H), 2.24-2.12 (m, 3H), 1.84-1.71 (in, 4H).

### Tert-butyl 4-(4-bromo-5-chloro-2-fluorophenyl)-3,3-difluoropiperidine-1-carboxylate (Intermediate KL)

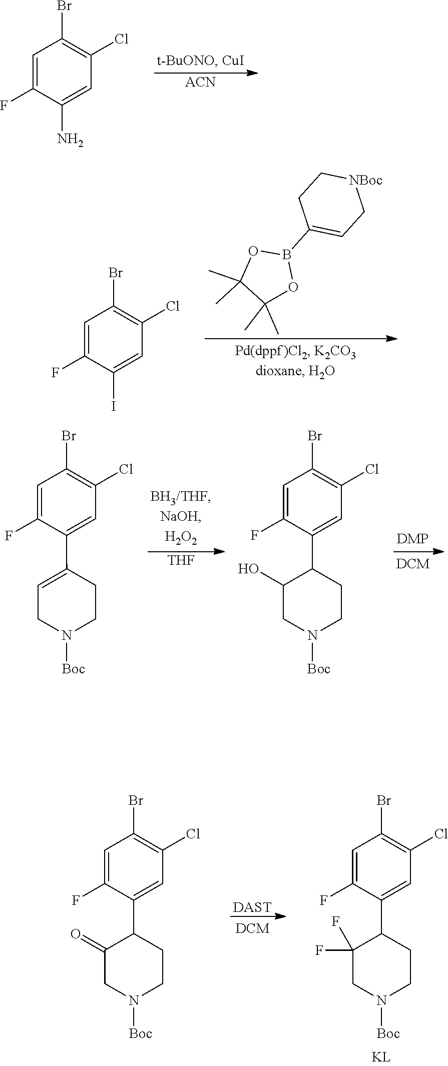

### Step 1—1-Bromo-2-chloro-5-fluoro-4-iodobenzene

To a solution of 4-bromo-5-chloro-2-fluoro-aniline (15 g, 67 mmol, CAS #116369-24-5) in ACN (20 mL) was added tert-butyl nitrite (10.3 g, 100 mmol, 11.9 mL) and CuI (15.2 g, 80.1 mmol). The mixture was then stirred at 50° C. for 2 hrs. On completion, the mixture was concentrated in vacuo to give the crude residue. The residue was purified by column chromatography (SiO₂, Petroleum ether:Ethyl acetate=1/0) to give the title compound (7 g, 31% yield) as a brown solid. ¹H NMR (400 MHz, DMSO-d₆) δ=8.16-8.13 (m, 1H), 7.84-7.80 (m, 1H).

### Step 2—Tert-butyl 4-(4-bromo-5-chloro-2-fluorophenyl)-3,6-dihydropyridine-1 (2H)-carboxylate

A mixture of 1-bromo-2-chloro-5-fluoro-4-iodobenzene (6.5 g, 19 mmol), tert-butyl 4-(4,4,5,5-tetramethyl-1,3,2-dioxaborolan-2-yl)-3,6-dihydropyridine-1 (2H)-carboxylate (5.99 g, 19.3 mmol, CAS #286961-14-6), Pd(dppf)Cl₂ (1.42 g, 1.94 mmol), K₂CO₃ (8.04 g. 58.1 mmol) in dioxane (100 mL) and H₂O (10 mL) was degassed and purged with N₂ three times. Then the mixture was stirred at 90° C. for 2 hrs under N₂ atmosphere. On completion, the mixture was quenched with water (100 mL) and extracted with ethyl acetate (2×100 mL). The combined organic layers were washed with brine (200 mL) and dried over anhydrous sodium sulfate, filtered and the filtrate was concentrated in vacuo to give the crude residue. The residue was purified by column chromatography (SiO₂, Petroleum ether:Ethyl acetate=1/0) to give the title compound (4.4 g, 58% yield) as a yellow oil. LC-MS (ESI⁺) m/z 335.8 (M−55)⁺.

### Step 3—Tert-butyl 4-(4-bromo-5-chloro-2-fluorophenyl)-3-hydroxypiperidine-1-carboxylate

A mixture of tert-butyl 4-(4-bromo-5-chloro-2-fluorophenyl)-3,6-dihydropyridine-1 (2H)-carboxylate (2 g, 5 mmol) in THF (16 mL) was added BH₃·THF (1 M, 7.68 mL) added 0° C. Then the mixture was stirred at 25° C. for 16 hrs. Next, NaOH (511 mg, 12.8 mmol) in H₂O (2 mL) was added dropwise at 0° C., and the mixture was stirred for 0.5 hrs. Finally, H₂O₂ (1.45 g, 12.8 mmol, 1.23 mL, 30% solution) was added, and the mixture was stirred at 50° C. for 2.5 hrs under N₂ atmosphere. On completion, the mixture was quenched with Na₂S₂O₃ (20 mL) and extracted with ethyl acetate (2×20 mL). The combined organic layers were washed with brine (40 mL) and dried over anhydrous sodium sulfate, filtered and the filtrate was concentrated in vacuo to give the crude residue. The residue was purified by column chromatography (SiO₂, Petroleum ether:Ethyl acetate=1/0 to 5/1) to give the title compound (1.25 g, 60% yield) as a white solid. LC-MS (ESI⁺) m/z 353.1 (M+H)⁺.

### Step 4—Tert-butyl 4-(4-bromo-5-chloro-2-fluorophenyl)-3-oxopiperidine-1-carboxylate

To a solution of tert-butyl 4-(4-bromo-5-chloro-2-fluorophenyl)-3,6-dihydropyridine-1(2H)-carboxylate (1.25 g, 3.06 mmol) in DCM (10 mL) was added DMP (1.95 g. 4.59 mmol. 1.42 mL) at 0° C. Then the mixture was stirred at 25° C. for 3 hrs. On completion, the mixture was quenched with Na₂S₂O₃ (10 mL) and extracted with dichloromethane (2×10 mL). The combined organic layers were washed with brine (20 mL) and dried over anhydrous sodium sulfate, filtered and the filtrate was concentrated in vacuo to give the crude residue. The residue was purified by reversed-phase HPLC (0.1% FA condition) to give the title compound (1 g, 80% yield) as a yellow oil. LC-MS (ESI⁺) m/z 351.9 (M−55)⁺.

### Step 5—Tert-butyl 4-(4-bromo-5-chloro-2-fluorophenyl)-3,3-difluoropiperidine-1-carboxylate

A mixture of tert-butyl 4-(4-bromo-5-chloro-2-fluorophenyl)-3-oxopiperidine-1-carboxylate (900 mg, 2.21 mmol), and DAST (713 mg, 4.43 mmol, 584 μL) in DCM (20 mL) was degassed and purged with N₂ three times at 0° C. Then the mixture was stirred at 25° C. for 12 hrs under N₂ atmosphere. On completion, the mixture was quenched with NaHCO₃ (10 mL) and extracted with dichloromethane (2×10 mL). The combined organic layers were washed with brine (20 mL) and dried over anhydrous sodium sulfate, filtered and the filtrate was concentrated in vacuo to give the crude residue. The residue was purified by reversed-phase HPLC (0.1% FA condition) to give the title compound (500 mg. 53% yield) as a colorless oil. LC-MS (ESI⁺) m/z 373.9 (M+H)⁺.

### Tert-butyl (S)-4-(4-(6-(1-(3-(1H-pyrazol-1-yl)propanoyl)-1,2,5,6-tetrahydropyridin-3-yl)-2-(dimethylcarbamoyl)-7-fluoro-1H-indol-4-yl)-5-chloro-2-fluorophenyl)-3,3-difluoropiperidine-1-carboxylate (Intermediate KM) & tert-butyl (R)-4-(4-(6-(1-(3-(1H-pyrazol-1-yl)propanoyl)-1,2,5,6-tetrahydropyridin-3-yl)-2-(dimethylcarbamoyl)-7-fluoro-1H-indol-4-yl)-5-chloro-2-fluorophenyl)-3,3-difluoropiperidine-1-carboxylate (Intermediate KN)

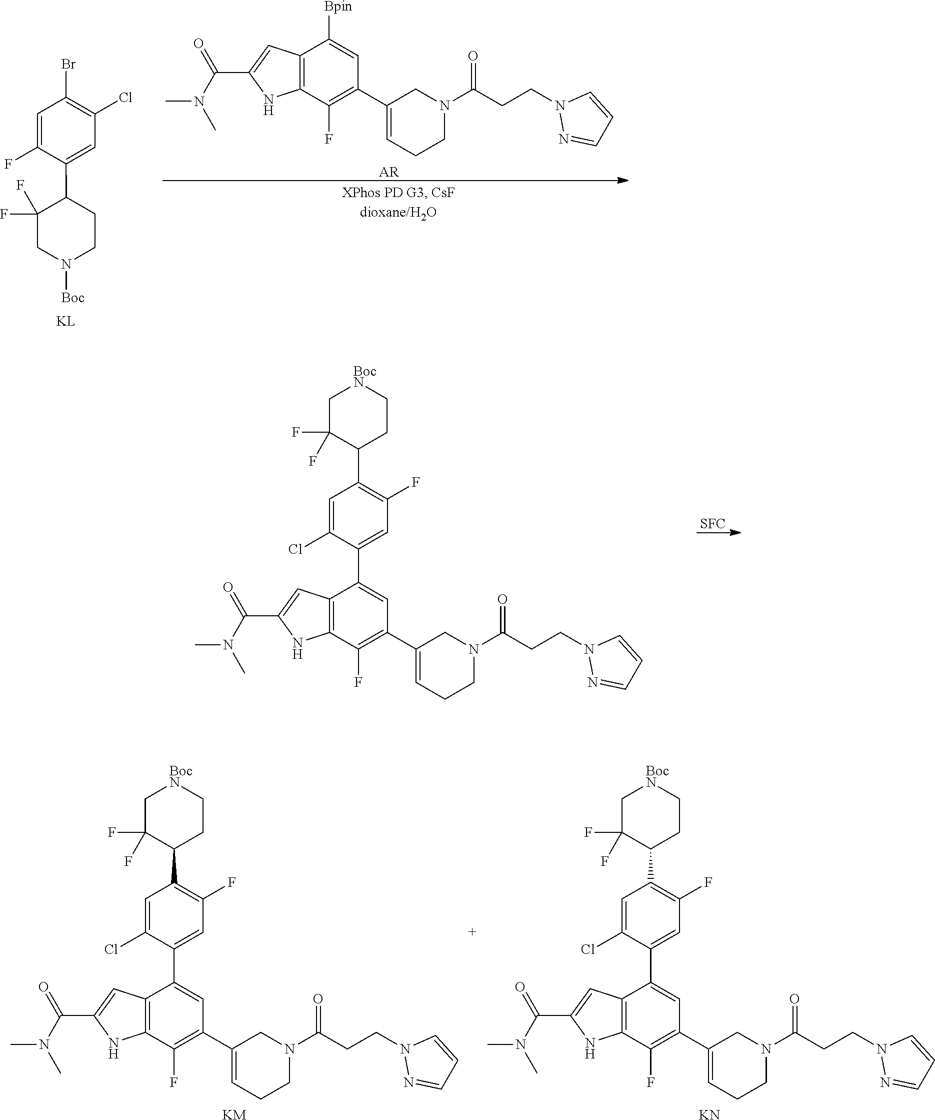

### Step 1—Tert-butyl 4-(4-(6-(1-(3-(TH-pyrazol-1-yl)propanoyl)-1,2,5,6-tetrahydropyridin-3-yl)-2-(dimethylcarbamoyl)-7-fluoro-1H-indol-4-yl)-5-chloro-2-fluorophenyl)-3,3-difluoropiperidine-1-carboxylate

A mixture of tert-butyl 4-(4-bromo-5-chloro-2-fluorophenyl)-3,3-difluoropiperidine-1-carboxylate (200 mg, 466 μmol, Intermediate KL), 6-(1-(3-(1H-pyrazol-1-yl)propanoyl)-1,2,5,6-tetrahydropyridin-3-yl)-7-fluoro-N,N-dimethyl-4-(4,4,5,5-tetramethyl-1,3,2-dioxaborolan-2-yl)-1H-indole-2-carboxamide (249 mg, 466 μmol, Intermediate AR). Pd(dppf)Cl₂·CH₂Cl₂ (38.1 mg, 46.6 μmol), and K₂CO₃ (193 mg, 1.40 mmol) in dioxane (10 mL) and H₂O (1 mL) was degassed and purged with N₂ three times. Then the mixture was stirred at 80° C. for 1 hr under N₂ atmosphere. On completion, the mixture was quenched with water (20 mL) and extracted with ethyl acetate (2×20 mL). The combined organic layers were washed with brine (40 mL) and dried over anhydrous sodium sulfate, filtered and the filtrate was concentrated in vacuo to give the crude residue. The residue was purified by reversed-phase HPLC (0.1% FA condition) to give the title compound (300 mg, 84% yield) as a yellow oil. LC-MS (ESI⁺) m/z 758.3 (M+H)⁺.

### Step 2—Tert-butyl (S)-4-(4-(6-(1-(3-(1H-pyrazol-1-yl)propanoyl)-1,2,5,6-tetrahydropyridin-3-yl)-2-(dimethylcarbamoyl)-7-fluoro-1H-indol-4-yl)-5-chloro-2-fluorophenyl)-3,3-difluoropiperidine-1-carboxylate and tert-butyl (R)-4-(4-(6-(1-(3-(1H-pyrazol-1-yl)propanoyl)-1,2,5,6-tetrahydropyridin-3-yl)-2-(dimethylcarbamoyl)-7-fluoro-1H-indol-4-yl)-5-chloro-2-fluorophenyl)-3,3-difluoropiperidine-1-carboxylate

Tert-butyl 4-(4-(6-(1-(3-(1H-pyrazol-1-yl)propanoyl)-1,2,5,6-tetrahydropyridin-3-yl)-2-(dimethylcarbamoyl)-7-fluoro-1H-indol-4-yl)-5-chloro-2-fluorophenyl)-3,3-difluoropiperidine-1-carboxylate was purified by SFC (column: DAICEL CHIRALCEL OD (250 mm*30 mm, 10 um); mobile phase: [CO₂-MeOH (0.1% NH₃H₂O)]; B %:45%, isocratic elution mode) to give the first eluting isomer tert-butyl (S)-4-(4-(6-(1-(3-(1H-pyrazol-1-yl)propanoyl)-1,2,5,6-tetrahydropyridin-3-yl)-2-(dimethylcarbamoyl)-7-fluoro-1H-indol-4-yl)-5-chloro-2-fluorophenyl)-3,3-difluoropiperidine-1-carboxylate (80 mg, 27% yield) and the second eluting isomer tert-butyl (R)-4-(4-(6-(1-(3-(1H-pyrazol-1-yl)propanoyl)-1,2,5,6-tetrahydropyridin-3-yl)-2-(dimethylcarbamoyl)-7-fluoro-1H-indol-4-yl)-5-chloro-2-fluorophenyl)-3,3-difluoropiperidine-1-carboxylate (80 mg, 27% yield) as a white solids. LC-MS (ESI⁺) m/z 757.3 (M+H)⁺ for both isomers. Absolute stereochemistry of the enantiomers was assigned arbitrarily.

### (R)-6-(1-(3-(1H-pyrazol-1-yl)propanoyl)-1,2,5,6-tetrahydropyridin-3-yl)-4-(2-chloro-4-(3,3-difluoropiperidin-4-yl)-5-fluorophenyl)-7-fluoro-N,N-dimethyl-1H-indole-2-carboxamide (Intermediate KO)

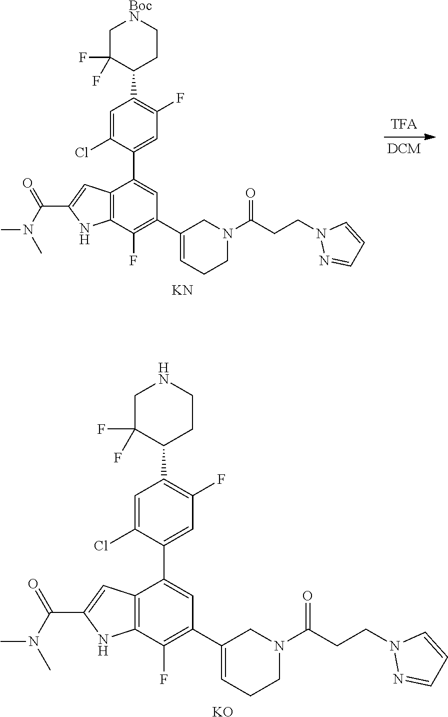

To a solution of tert-butyl (R)-4-(4-(6-(1-(3-(1H-pyrazol-1-yl)propanoyl)-1,2,5,6-tetrahydropyridin-3-yl)-2-(dimethylcarbamoyl)-7-fluoro-1H-indol-4-yl)-5-chloro-2-fluorophenyl)-33-difluoropiperidine-1-carboxylate (80.00 mg, 105 μmol, Intermediate KN) in DCM (10 mL) was added TFA (12.0 mg, 105 μmol, 7.85 L). The mixture was then stirred at 25° C. for 1 hr. On completion, the mixture was concentrated in vacuo to give the title compound (80 mg) as yellow oil. LC-MS (ESI⁺) m/z 657.4 (M+H)⁺.

### 3-(4-(4-(4-(4-Chloro-3-fluorophenyl)piperazin-1-yl)piperidin-1-yl)-3-fluorophenyl)piperidine-2,6-dione (Intermediate KP)

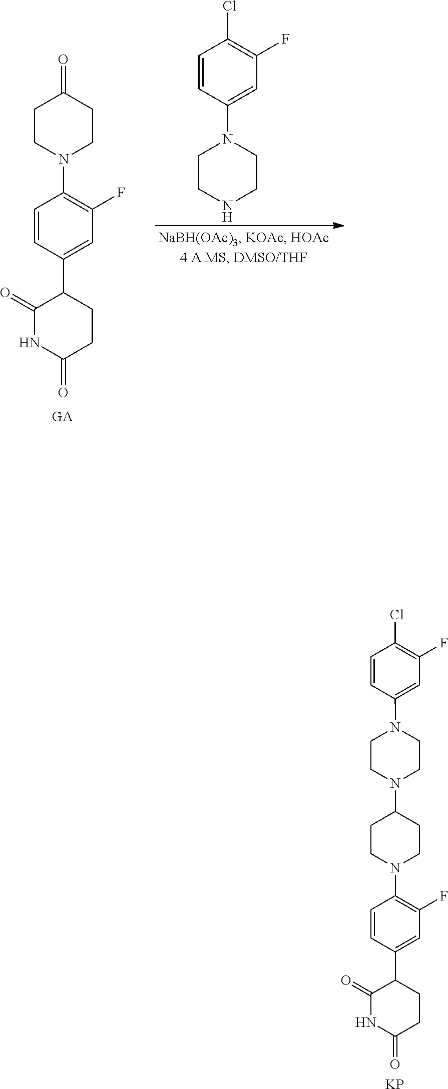

To a solution of 1-(4-chloro-3-fluoro-phenyl)piperazine (346 mg, 1.38 mmol, HCl, CAS #565473-01-0) in DMSO (5 mL), THF (5 mL) was added 4 Å molecular (300 mg, 985 μmol) and KOAc (193 mg, 1.97 mmol) until the pH was 6. Then 3-[3-fluoro-4-(4-oxo-1-piperidyl)phenyl]piperidine-2,6-dione (300 mg, 985 μmol, Intermediate GA) and HOAc (177 mg, 2.96 mmol, 169 μL) was added until the pH was 4. The mixture was then stirred at 60° C. for 12 hrs. Next, NaBH(OAc)₃ (522 mg, 2.46 mmol) was added at 0° C., then the mixture was stirred at 25° C. for 2 hrs. On completion, to the reaction mixture was added water (5 mL) and extracted with ethyl acetate (3×20 mL). The combined organic layers were dried over anhydrous sodium sulfate, filtered and the filtrate was concentrated in vacuo to give the crude residue. The residue was purified by column chromatography (SiO₂, Petroleum ether/Ethyl acetate=1/1 to 0/1, THF:EA=1:1 to 1:0) to give the title compound (150 mg, 15% yield) as a yellow solid. LC-MS (ESI⁺) m/z 503.2 (M+H)⁺.

### 2,6-Bis(benzyloxy)-5′,6′-difluoro-3,3′-bipyridine (Intermediate KO)

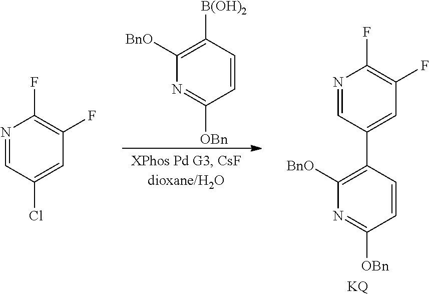

A mixture of 5-chloro-2,3-difluoro-pyridine (2 g, 10 mmol, CAS #89402-43-7), (2,6-dibenzyloxy-3-pyridyl)boronic acid (4.93 g, 14.7 mmol, CAS #2096339-92-1), XPhos Pd G3 (1.13 g, 1.34 mmol), and CsF (6.10 g, 40.1 mmol) in dioxane (15 mL) and H₂O (3 mL) was degassed and purged with N₂ three times. Then the mixture was stirred at 80° C. for 1 hr under N₂ atmosphere. On completion, the reaction mixture was concentrated under reduced pressure to give a residue. The crude residue was purified by column chromatography (SiO₂, Petroleum ether/Ethyl acetate=20/1) to give the title compound (5 g, 68% yield) as a yellow solid. LC-MS (ESI⁺) m/z 405.2 (M+H)⁺.

### 3-(6-(4-(2,3-Difluoro-4-(4,4,5,5-tetramethyl-1,3,2-dioxaborolan-2-yl)phenyl)piperazin-1-yl)-5-fluoropyridin-3-yl)piperidine-2,6-dione (Intermediate KR)

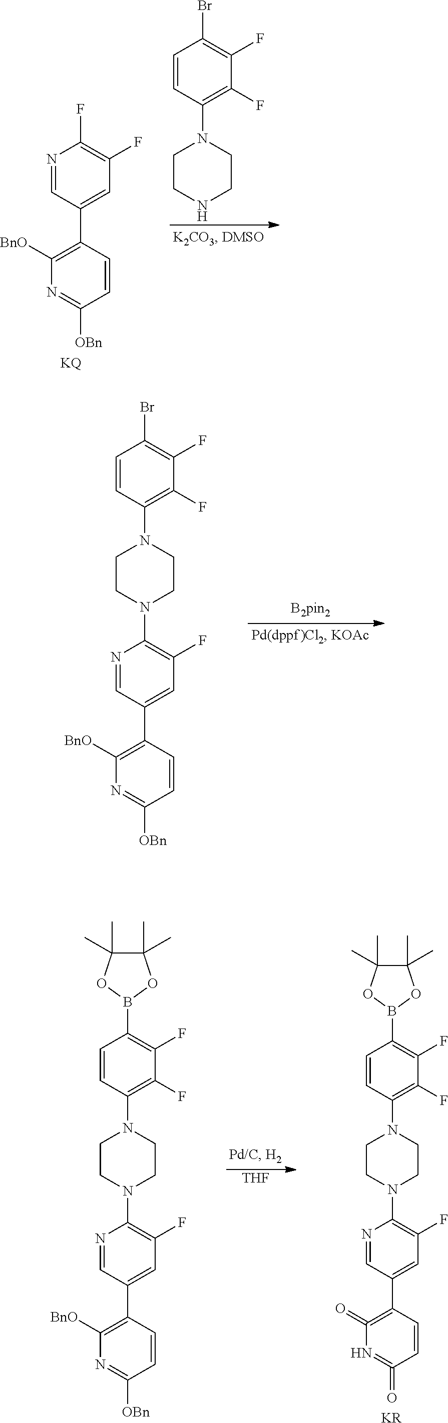

### Step 1—2,6-Bis(benzyloxy)-6′-(4-(4-bromo-2,3-difluorophenyl)piperazin-1-yl)-5′-fluoro-3,3′-bipyridine

To a solution of 2,6-dibenzyloxy-3-(5,6-difluoro-3-pyridyl)pyridine (1 g, 3 mmol, Intermediate KQ) and 1-(4-bromo-2,3-difluoro-phenyl)piperazine (930 mg, 2.97 mmol, CAS #1121610-13-6) in DMSO (10 mL) was added K₂CO₃ (1.03 g, 7.42 mmol). The mixture was then stirred at 120° C. for 3 hrs. On completion, the reaction mixture was quenched by addition of H₂O (10 mL), and then extracted with EtOAc (20 mL×3). The combined organic layers were washed with brine (20 mL×3), dried over Na₂SO₄, filtered and concentrated under reduced pressure to give a residue. The crude residue was purified by column chromatography (1.1 g, 63% yield) to give the title compound (5 g, 68% yield) as a white solid. LC-MS (ESI⁺) m/z 661.2 (M+H)⁺.

### Step 2—2,6-Bis(benzyloxy)-6′-(4-(2,3-difluoro-4-(4,4,5,5-tetramethyl-1,3,2-dioxaborolan-2-yl)phenyl)piperazin-1-yl)-5′-fluoro-3,3′-bipyridine

A mixture of 1-(4-bromo-2,3-difluoro-phenyl)-4-[5-(2,6-dibenzyloxy-3-pyridyl)-3-fluoro-2-pyridyl]piperazine (500 mg, 756 μmol), 4,4,5,5-tetramethyl-2-(4,4,5,5-tetramethyl-1,3,2-dioxaborolan-2-yl)-1,3,2-dioxaborolane (384 mg, 1.51 mmol), Pd(dppf)Cl₂·CH2Cl₂ (123 mg, 151 μmol), and KOAc (223 mg, 2.27 mmol) in dioxane (5 mL) was degassed and purged with N₂ three times. Then the mixture was stirred at 80° C. for 1 hr under N₂ atmosphere. On completion, the reaction mixture was concentrated under reduced pressure to remove solvent give a residue. The crude residue was purified by column chromatography (SiO₂, Petroleum ether/Ethyl acetate=30/1 to 5/1) to give the title compound (200 mg, 35% yield) as a white solid. LC-MS (ESI⁺) m/z 708.2 (M+H)⁺.

### Step 3—3-(6-(4-(2,3-Difluoro-4-(4,4,5,5-tetramethyl-1,3,2-dioxaborolan-2-yl)phenyl)piperazin-1-yl)-5-fluoropyridin-3-yl)piperidine-2,6-dione

A mixture of 1-[5-(2,6-dibenzyloxy-3-pyridyl)-3-fluoro-2-pyridyl]-4-[2,3-difluoro-4-(4,4,5,5-tetramethyl-1,3,2-dioxaborolan-2-yl)phenyl]piperazine (200 mg, 282 μmol), Pd/C (200 mg, 187 μmol, 10 wt %) in THF (5 mL) was degassed and purged with H₂ three times. Then the mixture was stirred at 30° C. for 12 h under H₂ atmosphere. On completion, the reaction mixture was filtered and concentrated under reduced pressure to give the title compound (160 mg) as a white solid. LC-MS (ESI⁺) m/z 531.2 (M+H)⁺.

### 4-(4-chloro-2-fluorophenyl)piperidine (Intermediate KS)

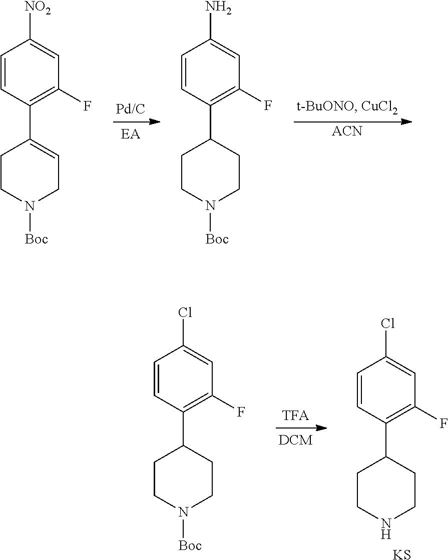

### Step 1—Tert-butyl 4-(2-fluoro-4-nitrophenyl)-5,6-dihydropyridine-1 (2H)-carboxylate

A mixture of tert-butyl 4-(4,4,5,5-tetramethyl-1,3,2-dioxaborolan-2-yl)-3,6-dihydro-2H-pyridine-1-carboxylate (11.8 g. 38.1 mmol). 1-bromo-2-fluoro-4-nitro-benzene (7 g. 30 mmol, CAS #185331-69-5), Pd(dppf)Cl₂ (2.33 g, 3.18 mmol), and K₂CO₃ (13.1 g, 95.4 mmol) in dioxane (40 mL) and H₂O (4 mL) was degassed and purged with N₂ three times. Then the mixture was stirred at 80° C. for 2 h under N₂ atmosphere. On completion, the reaction mixture was diluted with water (100 mL) and extracted with dichloromethane (2×100 mL). The combined organic layers were washed with brine (200 mL) and dried over anhydrous sodium sulfate, filtered and the filtrate was concentrated in vacuo to give the crude residue. The residue was purified by column chromatography (SiO₂, Petroleum ether:Ethyl acetate=1/0 to 5/1) to give the title compound (14 g) as yellow solid. LC-MS (ESI⁺) m/z 266.9 (M−55)⁻.

### Step 2—Tert-butyl 4-(4-amino-2-fluorophenyl)piperidine-1-carboxylate

To a solution of tert-butyl 4-(2-fluoro-4-nitrophenyl)-3,6-dihydropyridine-1 (2H)-carboxylate (2 g, 6.20 mmol) in EtOAc (20 mL) was added Pd/C (10 wt %, 2 g) under N₂ atmosphere. The suspension was degassed and purged with H₂ for 3 times. The mixture was then stirred under H₂ (15 psi) at 25° C. for 15 hrs. On completion, the mixture was filtered and the filtrate was concentrated in vacuo to give the title compound (2 g) as yellow solid. LC-MS (ESI⁺) m/z 238.9 (M−55)⁺.

### Step 3—Tert-butyl 4-(4-chloro-2-fluorophenyl)piperidine-1-carboxylate

To a solution of tert-butyl 4-(4-amino-2-fluorophenyl)piperidine-1-carboxylate (1.8 g, 6.1 mmol) in ACN (20 mL) was added tert-butyl nitrite (945 mg, 9.17 mmol, 1.09 mL) and CuCl₂ (986 mg, 7.34 mmol). The mixture was then stirred at 25° C. for 0.5 hrs. On completion, the mixture was quenched with water (20 mL) and extracted with ethyl acetate (2×20 mL). The combined organic layers were washed with brine (40 mL) and dried over anhydrous sodium sulfate, filtered and the filtrate was concentrated in vacuo to give the title compound (2 g) as a yellow oil. LC-MS (ESI⁺) m/z 258.0 (M−55)⁺.

### Step 4—4-(4-Chloro-2-fluorophenyl)piperidine

To a solution of tert-butyl 4-(4-chloro-2-fluorophenyl)piperidine-1-carboxylate (2 g, 6.37 mmol) in DCM (10 mL) was added TFA (1.54 g, 1 mL). The mixture was then stirred at 25° C. for 1 hr. On completion, the mixture was concentrated in vacuo and purified by reversed-phase HPLC (0.1% FA condition) to give the title compound (1 g, 73% yield) as a white solid. LC-MS (ESI⁺) m/z 214.0 (M−100)⁺.

### 2,6-Bis(benzyloxy)-3-(4-(1-(4-chloro-2,3-difluorophenyl)piperidin-4-yl)-3-fluorophenyl)pyridine (Intermediate KT)

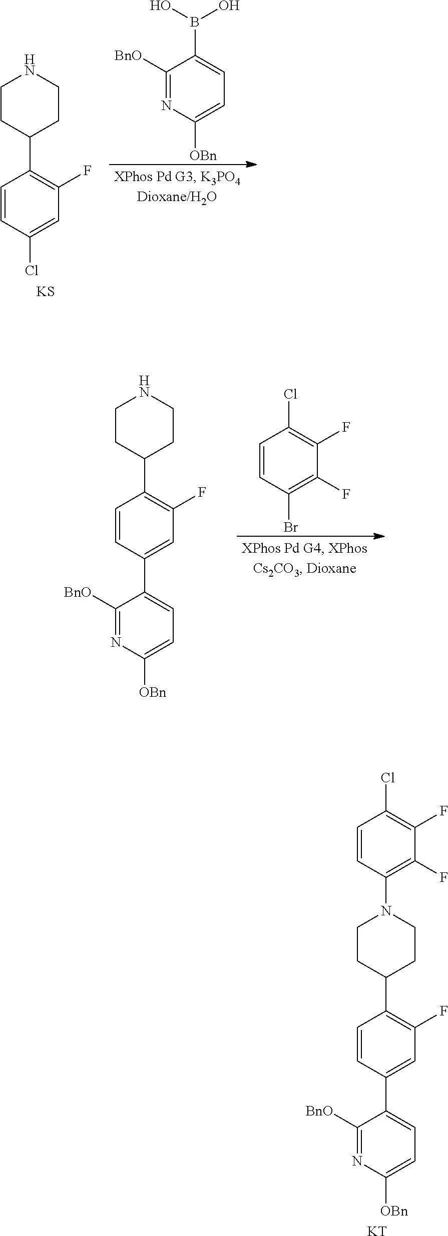

### Step 1—2,6-Bis(benzyloxy)-3-(3-fluoro-4-(piperidin-4-yl)phenyl)pyridine

A mixture of 4-(4-chloro-2-fluorophenyl)piperidine (I g, 5 mmol, Intermediate KS), (2,6-bis(benzyloxy)pyridin-3-yl)boronic acid (1.57 g, 4.68 mmol, CAS #2096339-92-1), XPhos Pd G3 (396 mg, 467 μmol), and K₃PO₄ (2.98 g, 14.0 mmol) in dioxane (40 mL) and H₂O (4 mL) was degassed and purged with N₂ three times. Then the mixture was stirred at 80° C. for 2 hrs under N₂ atmosphere. The mixture was quenched with water (20 mL) and extracted with ethyl acetate (2×20 mL). The combined organic layers were washed with brine (40 mL) and dried over anhydrous sodium sulfate, filtered and the filtrate was concentrated in vacuo to give the crude residue. The residue was purified by reversed-phase HPLC (0.1% FA condition) to give the title compound (1.2 g, 55% yield) as a brown solid. LC-MS (ESI⁺) m/z 469.2 (M+H)⁺.

### Step 2—2,6-Bis(benzyloxy)-3-(4-(1-(4-chloro-2,3-difluorophenyl)piperidin-4-yl)-3-fluorophenyl)pyridine

A mixture of 2,6-bis(benzyloxy)-3-(3-fluoro-4-(piperidin-4-yl)phenyl)pyridine (650 mg, 1.39 mmol), 1-bromo-4-chloro-2,3-difluorobenzene (315.50 mg, 1.39 mmol, CAS #1000574-47-9), XPhos (66.13 mg, 138.72 μmol), Xphos Pd G4 (119.37 mg, 138.72 μmol) and Cs₂CO₃ (1.36 g, 4.16 mmol) in dioxane (10 mL) was degassed and purged with N₂ three times. Then the mixture was stirred at 100° C. for 8 hrs under N₂ atmosphere. On completion, the mixture was quenched with water (20 mL) and extracted with ethyl acetate (2×20 mL). The combined organic layers were washed with brine (40 mL) and dried over anhydrous sodium sulfate, filtered and the filtrate was concentrated in vacuo to give the crude residue. The residue was purified by column chromatography (SiO₂, Petroleum ether:Ethyl acetate=1/0 to 10/1) to give the title compound (500 mg, 59% yield)) as a colorless oil. LC-MS (ESI⁺) m/z 615.2 (M+H)⁺.

### 3-(4-(1-(4-Bromo-2,3-difluorophenyl)piperidin-4-yl)-3-fluorophenyl)piperidine-2,6-dione (Intermediate KU)

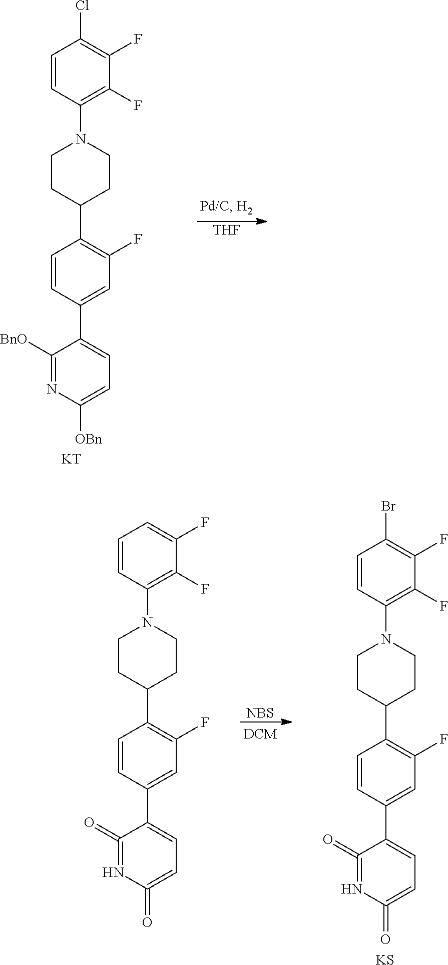

### Step 1—3-(4-(1-(2,3-Difluorophenyl)piperidin-4-yl)-3-fluorophenyl)piperidine-2,6-dione

To a solution of 2,6-bis(benzyloxy)-3-(4-(1-(4-chloro-2,3-difluorophenyl)piperidin-4-yl)-3-fluorophenyl)pyridine (500 mg, 812 μmol, Intermediate KT) in THE (5 mL) was added Pd/C (10 wt %, 500 mg) under N₂ atmosphere. The suspension was degassed and purged with H₂ three times. The mixture was then stirred under H₂ (15 psi) at 25° C. for 12 hrs. On completion, the mixture was filtered and the filtrate was concentrated in vacuo to give the crude residue. The residue was purified by reversed-phase HPLC (0.1% FA condition) to give the title compound (200 mg, 61% yield) as a white solid. LC-MS (ESI⁺) m/z 403.2 (M+H)⁺.

### Step 2—3-(4-(1-(4-Bromo-2,3-difluorophenyl)piperidin-4-yl)-3-fluorophenyl)piperidine-2,6-dione

To a solution of 3-(4-(1-(2,3-difluorophenyl)piperidin-4-yl)-3-fluorophenyl)piperidine-2,6-dione (200 mg, 497 μmol) in DCM (10 mL) was added NBS (88.4 mg, 497 μmol). The mixture was then stirred at 25° C. for 3 hrs. On completion, the mixture was quenched with water (10 mL) and extracted with dichloromethane (2×20 mL). The combined organic layers were washed with brine (20 mL) and dried over anhydrous sodium sulfate, filtered and the filtrate was concentrated in vacuo to give the crude residue. The residue was purified by reversed-phase HPLC (0.1% FA condition) to give the title compound (100 mg, 42% yield) as a white solid. LC-MS (ESI⁺) m/z 483.0 (M+H)⁺.

### 3-(4-(4-(4-(4-Chloro-2,5-difluorophenyl)piperazin-1-yl)piperidin-1-yl)-3-fluorophenyl)piperidine-2,6-dione (Intermediate KV)

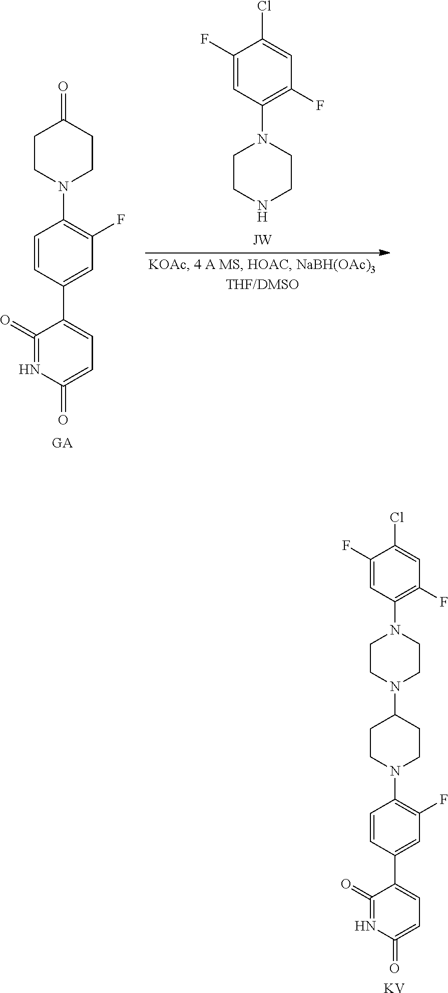

To a solution of 1-(4-chloro-2,5-difluorophenyl)piperazine (265 mg, 986 μmol, Intermediate JW) in THF (4 mL) and DMSO (4 mL) was added 4 Å molecular sieves (400 mg), and KOAc (121 mg, 1.23 mmol) until the pH 6-7. Then 3-[3-fluoro-4-(4-oxo-1-piperidyl)phenyl]piperidine-2,6-dione (250 mg, 822 μmol, Intermediate GA) in was added followed by HOAc (148 mg, 2.46 mmol, 141 μL) dropwise until the pH 3-4. The resulting mixture was stirred at 60° C. for 12 h, and then NaBH(OAc)₃ (696 mg, 3.29 mmol) in was added dropwise. The resulting mixture was stirred at 25° C. for 2 h. On completion, the reaction mixture was concentrated in vacuo to give the crude residue. The residue was purified by column chromatography (SiO₂, Petroleum ether/Ethyl acetate=2/1 to 0/1) to give the title compound (138 mg, 32% yield) as a yellow solid. LC-MS (ESI⁺) n/z 521.1 (M+H)⁺.

### 6-Bromo-4-chloro-7-fluoro-N,N-bis(methyl-d3)-1H-indole-2-carboxamide (Intermediate KW)

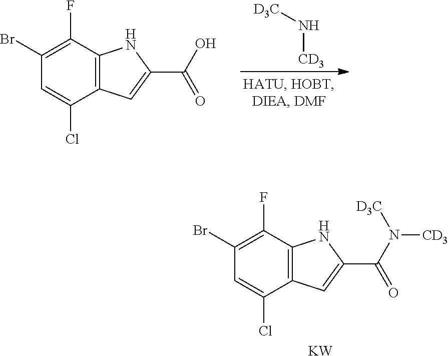

To a solution of 6-bromo-4-chloro-7-fluoro-1H-indole-2-carboxylic acid (1.3 g, 4.44 mmol, synthesized via Step 1 of Intermediate T) in DMF (15 mL) was added HATU (2.20 g, 5.78 mmol), DIEA (2.87 g, 22.2 mmol) and HOBt (901 mg, 6.67 mmol) at 0° C. Then the reaction was stirred at 25° C. for 20 mins, then 1,1,1-trideuterio-N-(trideuteriomethyl)methanamine; hydrochloride (701 mg, 8.00 mmol, CAS #53170-19-7) was added at 25° C. The reaction was stirred at 25° C. for 1 hr. On completion, the reaction mixture was filtered and the filtrate was concentrated in vacuo to give the crude residue. The crude product was triturated with H₂O (30 mL) at 25° C. for 0.5 hr, then the mixture was filtered and the filter cake was dried in vacuo to give the title compound (1.2 g, 83% yield) as a yellow solid. LC-MS (ESI⁺) m/z 324.8 (M−H)⁺.

### 6-(1-(3-(1H-pyrazol-1-yl)propanoyl)-1,2,5,6-tetrahydropyridin-3-yl)-7-fluoro-N,N-bis(methyl-d3)-4-(4,4,5,5-tetramethyl-1,3,2-dioxaborolan-2-yl)-1H-indole-2-carboxamide (Intermediate KX)

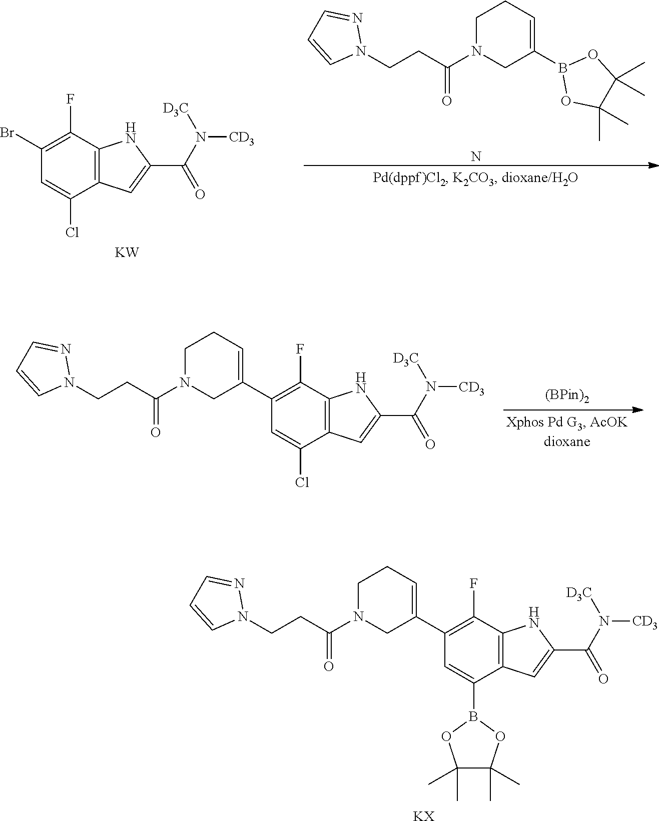

### Step 1—6-(1-(3-(1H-pyrazol-1-yl)propanoyl)-1,2,5,6-tetrahydropyridin-3-yl)-4-chloro-7-fluoro-N,N-bis(methyl-d3)-1H-indole-2-carboxamide

To a solution of 6-bromo-4-chloro-7-fluoro-N,N-bis(methyl-d3)-1H-indole-2-carboxamide (1.02 g, 3.13 mmol, Intermediate KW), 3-(1H-pyrazol-1-yl)-1-(5-(4,4,5,5-tetramethyl-1,3,2-dioxaborolan-2-yl)-3,6-dihydropyridin-1 (2H)-yl)propan-1-one (1.55 g, 4.69 mmol, Intermediate N) in dioxane (12 mL) and H₂O (2.5 mL) was added Pd(dppf)Cl₂·CH₂Cl₂ (256 mg, 313 μmol) and K₂CO₃ (1.30 g, 9.39 mmol) at 25° C. Then the reaction was stirred at 80° C. for 2 hrs under nitrogen atmosphere. On completion, the reaction mixture was filtered and the filtrate was concentrated in vacuo to give the crude residue. The residue was purified by column chromatography (SiO₂, Petroleum ether:Ethyl acetate=0/1) to give the title compound (0.8 g, 52% yield) as a yellow solid. LC-MS (ESI⁺) m/z 450.1 (M+H)⁺.

### Step 2—6-(1-(3-(1H-pyrazol-1-yl)propanoyl)-1,2,5,6-tetrahydropyridin-3-yl)-7-fluoro-N,N-bis(methyl-d3)-4-(4,4,5,5-tetramethyl-1,3,2-dioxaborolan-2-yl)-1H-indole-2-carboxamide

To a solution of 6-(1-(3-(1H-pyrazol-1-yl)propanoyl)-1,2,5,6-tetrahydropyridin-3-yl)-4-chloro-7-fluoro-N,N-bis(methyl-d3)-1H-indole-2-carboxamide (800 mg, 1.78 mmol) and 4,4,5,5-tetramethyl-2-(4,4,5,5-tetramethyl-1,3,2-dioxaborolan-2-yl)-1,3,2-dioxaborolane (903 mg, 3.56 mmol) in dioxane (10 mL) was added XPhos Pd G₃ (150 mg, 178 μmol) and AcOK (524 mg, 5.33 mmol) at 25° C. Then the reaction was stirred at 80° C. for 1 hr under nitrogen atmosphere. On completion, the reaction mixture was filtered and the filtrate was concentrated in vacuo to give the crude residue. The residue was purified by column chromatography (SiO₂, Petroleum ether:Ethyl acetate=0/1) to give the title compound (620 mg, 54% yield) as a yellow solid. LC-MS (ESI⁺) m/z 452.3 (M+H)⁺.

### 4-(1-(4-chloro-2,5-difluorophenyl)piperidin-4-yl)-5-fluoro-2-methoxyaniline (Intermediate KY)

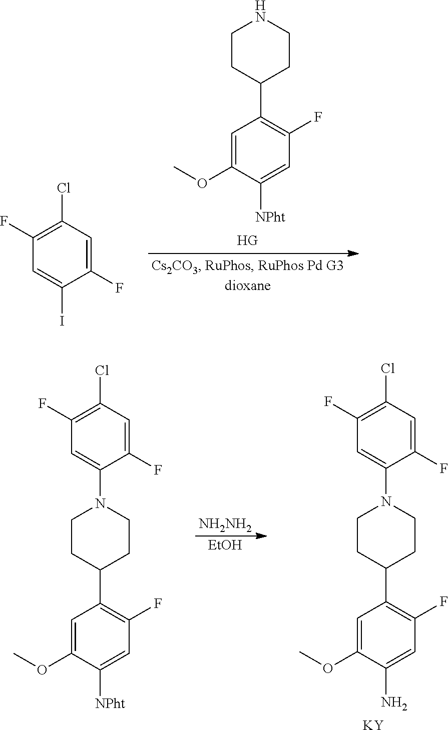

### Step 1—2-(4-(1-(4-Chloro-2,5-difluorophenyl)piperidin-4-yl)-5-fluoro-2-methoxyphenyl)isoindoline-1,3-dione

A solution of 1-chloro-2,5-difluoro-4-iodobenzene (3.00 g, 10.9 mmol, CAS #1097871-23-2), 2-(5-fluoro-2-methoxy-4-(piperidin-4-yl)phenyl)isoindoline-1,3-dione (3.87 g, 10.9 mmol, Intermediate HG), RuPhos (510 mg, 1.09 mmol), RuPhos Pd G3 (914 mg, 1.09 mmol), and Cs₂CO₃ (10.6 g, 32.7 mmol) in dioxane (60 mL) was stirred at 100° C. for 4 hrs. On completion, the reaction mixture was quenched with water (60 mL) and extracted with ethyl acetate (3×60 mL). The combined organic layers were washed with brine (60 mL) and dried over anhydrous sodium sulfate, filtered and the filtrate was concentrated in vacuo to give the crude residue. The residue was purified by column chromatography (SiO₂, Petroleum ether:Ethyl acetate=100/1 to 10/1) to give the title compound (700 mg) as a light yellow solid. LC-MS (ESI⁺) m/z 500.9 (M+H)⁺.

### Step 2—4-(1-(4-Chloro-2,5-difluorophenyl)piperidin-4-yl)-5-fluoro-2-methoxyaniline

A solution of 2-(4-(1-(4-chloro-2,5-difluorophenyl)piperidin-4-yl)-5-fluoro-2-methoxyphenyl)isoindoline-1,3-dione (700 mg, 1.35 mmol) and NH₂NH₂·H₂O (794 mg, 13.4 mmol. 769 μL) in EtOH (8 mL) was stirred at 50° C. for 2 hrs under N₂ condition. On completion, the mixture was quenched with addition H₂O (3 mL), and then extracted with EA (3×5 mL). The residue was purified by column chromatography (SiO₂, Petroleum ether:Ethyl acetate=100/1 to 10/1) to give the title compound (540 mg, 87% yield) as a yellow oil. ¹H NMR (400 MHz, DMSO-d₆) δ=7.50 (dd, J=7.2, 12.0 Hz, 1H), 7.11 (dd, J=7.6, 11.2 Hz, 1H), 6.69 (d, J=7.2 Hz, 1H), 6.38 (d, J=12.0 Hz, 1H), 4.83 (s, 2H), 3.75 (s, 3H), 3.47 (d, J=12.0 Hz, 2H), 2.81-2.76 (m, 2H), 1.85 (dq, J=3.2, 12.0 Hz, 2H), 1.79-1.73 (m, 2H).

### 3-((4-(1-(4-Chloro-2,5-difluorophenyl)piperidin-4-yl)-5-fluoro-2-methoxyphenyl)amino)piperidine-2,6-dione (Intermediate KZ)

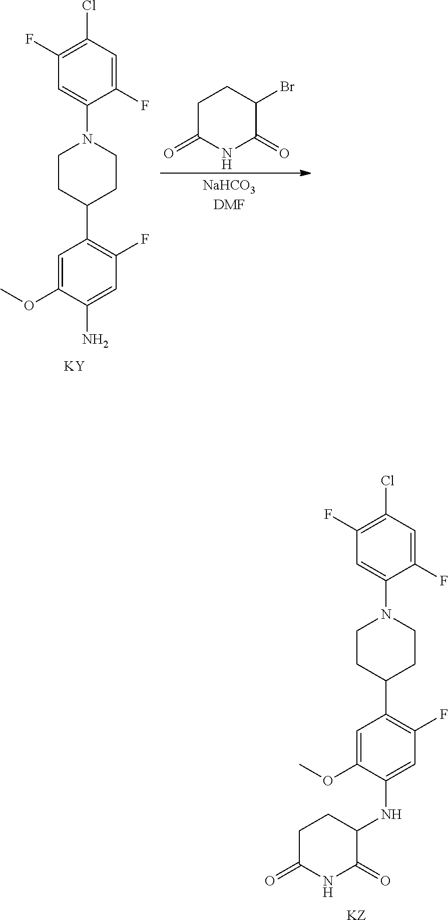

A solution of 4-(I-(4-chloro-2,5-difluorophenyl)piperidin-4-yl)-5-fluoro-2-methoxyaniline (540 mg, 1.46 mmol, Intermediate KY), 3-bromopiperidine-2,6-dione (559 mg, 2.91 mmol, CAS #62595-74-8), and NaHCO₃ (611 mg, 7.28 mmol, 283 L) in DMF (10 mL) was stirred at 80° C. for 24 hrs. On completion, the crude residue was purified by reversed-phase HPLC (0.8 g/L ammonium bicarbonate), then the residue was purified by column chromatography (SiO₂. Petroleum ether:Ethyl acetate=100/1 to 2/1) give the title compound (220 mg) as a green solid. LC-MS (ESI⁺) m/z 482.1 (M+H)⁺.

### 3-(4-(4-(4-(4-Bromo-2,3-difluorophenyl)piperazin-1-yl)piperidin-1-yl)-3-fluorophenyl)piperidine-2,6-dione (Intermediate LA)

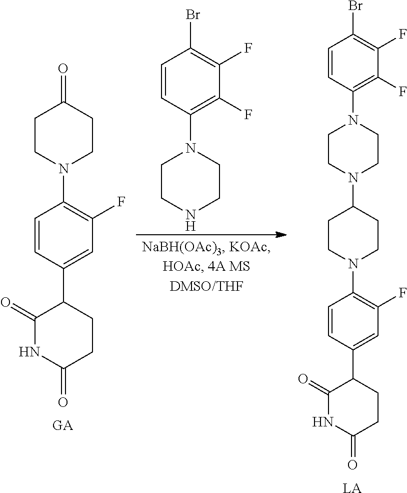

To a solution of 1-(4-bromo-2,3-difluoro-phenyl)piperazine (327 mg, 1.18 mmol, CAS #1121610-13-6) in THF (5 mL), DMSO (5 mL) was added 4 Å MS (500 mg) and KOAc (193 mg, 1.97 mmol) until pH 6, then was added 3-[3-fluoro-4-(4-oxo-1-piperidyl)phenyl]piperidine-2,6-dione (300 mg, 985 μmol, Intermediate GA), and HOAc (177 mg, 2.96 mmol) until the pH was 4. The mixture was then stirred at 60° C. for 12 hrs. Next, NaBH(OAc)₃ (522 mg, 2.46 mmol) was added at 0° C., then the mixture was stirred at 25° C. for 2 hrs. On completion, to the reaction mixture was added water (5 mL) and extracted with ethyl acetate (3×20 mL). The combined organic layers were dried over anhydrous sodium sulfate, filtered and the filtrate was concentrated in vacuo to give the crude residue. The residue was purified by column chromatography (SiO₂, Petroleum ether/Ethyl acetate=1/1 to 0/1, THF:EA=1:1 to 1:0) to give the title compound (150 mg, 15% yield) as a yellow solid. LC-MS (ESI⁺) m/z 565.1 (M+H)⁺.

### 1-(4-Chloro-2,5-difluorophenyl)piperidin-4-one (Intermediate LB)

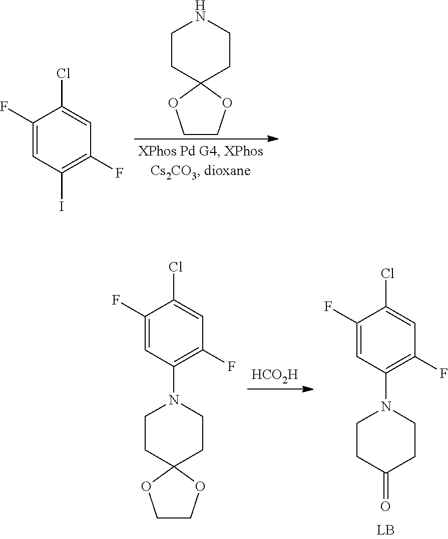 [SMILES: Fc1cc(I)c(F)cc1Cl ; C1CC2(CCN1)OCCO2 ; Fc1cc(N2CCC3(CC2)OCCO3)c(F)cc1Cl ; "HCO2H" ; O=C1CCN(c2cc(F)c(Cl)cc2F)CC1]

### Step 1—8-(4-chloro-2,5-difluorophenyl)-1,4-dioxa-8-azaspiro[4.5]decane

To a solution of 1,4-dioxa-8-azaspiro[4.5]decane (2.61 g, 18.2 mmol, 2.34 mL, CAS #177-11-7) and 1-chloro-2,5-difluoro-4-iodo-benzene (5 g, 18.22 mmol, CAS #1097871-23-2) in dioxane (50 mL) was added Xphos Pd G4 (1.57 g, 1.82 mmol), Cs₂CO₃ (17.81 g, 54.6 mmol) and XPhos (868 mg, 1.82 mmol) at 25° C. under nitrogen atmosphere. Then the mixture was stirred at 100° C. for 12 hrs under nitrogen atmosphere. On completion, the reaction mixture was concentrated in vacuo to give the crude residue. The residue was purified by column chromatography (SiO₂, Petroleum ether/Ethyl acetate=10/1 to 6/1) to give the title compound (4 g, 64% yield) as a yellow solid. LC-MS (ESI⁺) m/z 290.0 (M+H)⁺.

### Step 2—1-(4-Chloro-2,5-difluorophenyl)piperidin-4-one

To a solution of 8-(4-chloro-2,5-difluorophenyl)-1,4-dioxa-8-azaspiro[4.5]decane (2 g, 6.90 mmol) was added HCO₂H (331 mg, 6.90 mmol, 10 mL) at 25° C., then the reaction was stirred at 60° C. for 12 h. On completion, the reaction mixture was concentrated in vacuo to give the crude residue. The crude residue was purified by reversed-phase HPLC (0.8 g/L ammonium bicarbonate) to give the title compound (500 mg, 29% yield) as a brown solid. LC-MS (ESI⁺) m/z 246.0 (M+H)⁺.

### 3-(4-(4-(1-(4-Chloro-2,5-difluorophenyl)piperidin-4-yl)piperazin-1-yl)-3-fluorophenyl)piperidine-2,6-dione (Intermediate LC)

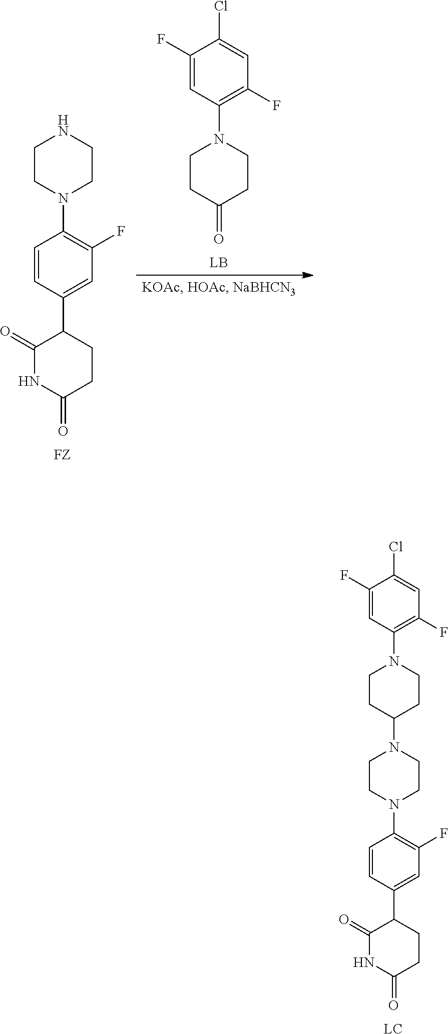

To a solution of 3-(3-fluoro-4-(piperazin-1-yl)phenyl)piperidine-2,6-dione (200 mg, 610 μmol, HC, Intermediate FZ) in DMSO (2 mL) and THF (1 mL) was added KOAc (89.8 mg, 915 μmol) at 25° C., and the mixture was stirred for 5 min. Then add HOAc (73.3 mg, 1.22 mmol, 67.0 μL) and 1-(4-chloro-2,5-difluorophenyl)piperidin-4-one (150 mg, 610 μmol, Intermediate LB) was added at 25° C., then the mixture was stirred for 1 h at 40° C. Next, NaBH₃CN (76.7 mg, 1.22 mmol) was added at 0° C., and the mixture was stirred at 25° C. for 1 hr. On completion, the reaction mixture was concentrated in vacuo to get the crude residue. The residue was purified by column chromatography (SiO₂, Petroleum ether/Ethyl acetate=1/1 to Ethyl acetate/THF=5/1) to give the title compound (100 mg, 18% yield) as a white solid. LC-MS (ESI⁺) m/z 521.2 (M+H)⁺.

### 3-(4-(4-(4-Chloro-3-methylphenyl)piperidin-1-yl)-3-methyl-1H-indazol-1-yl)piperidine-2,6-dione (Intermediate LD)

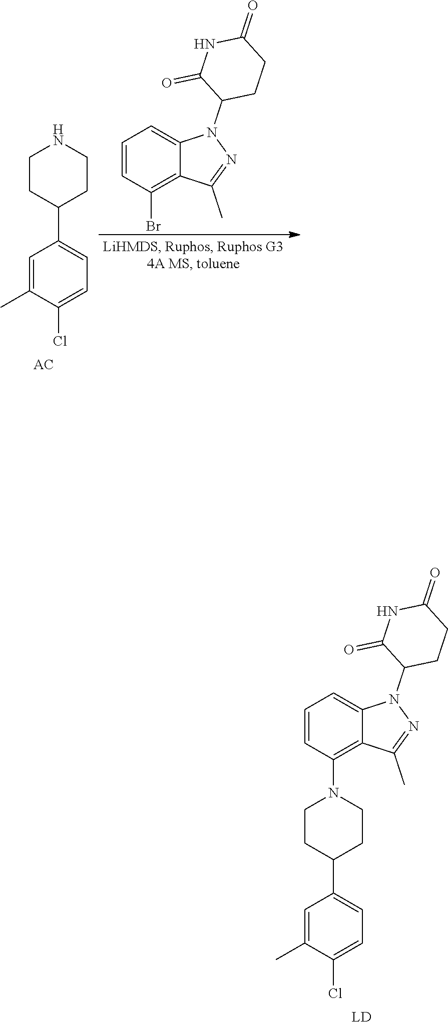

To a solution of 3-(4-bromo-3-methyl-indazol-1-yl)piperidine-2,6-dione (2.5 g, 7.76 mmol, synthesized via Step 1 of Intermediate AB) and 4-(4-chloro-3-methyl-phenyl)piperidine (1.63 g, 7.76 mmol, Intermediate AC) in toluene (50 mL) was added LiHMDS (1 M, 46.56 mL) and 4 Å molecular sieves (2 g, 7.76 mmol) under at 0° C. After 5 minutes, RuPhos (543 mg, 1.16 mmol) and RuPhos Pd G3 (649 mg, 776 μmol) was added, then the mixture was stirred at 100° C. for 1 hr. On completion, the reaction mixture was quenched with water (1000 mL) and extracted with ethyl acetate (3×100 mL). The combined organic layers were washed with brine (200 mL) and dried over anhydrous sodium sulfate, filtered and the filtrate was concentrated in vacuo. Then the mixture was purified by column chromatography (SiO₂. DCM/THF=3/1) to give the title compound (3.5 g, 80% yield) as a yellow solid. LC-MS (ESI⁺) m/z 451.2 (M+H)⁺.

### 3-(3-Fluoro-4-(4-(2-fluoro-4-(4,4,5,5-tetramethyl-1,3,2-dioxaborolan-2-yl)phenyl)piperidin-1-yl)phenyl)piperidine-2,6-dione (Intermediate LE)

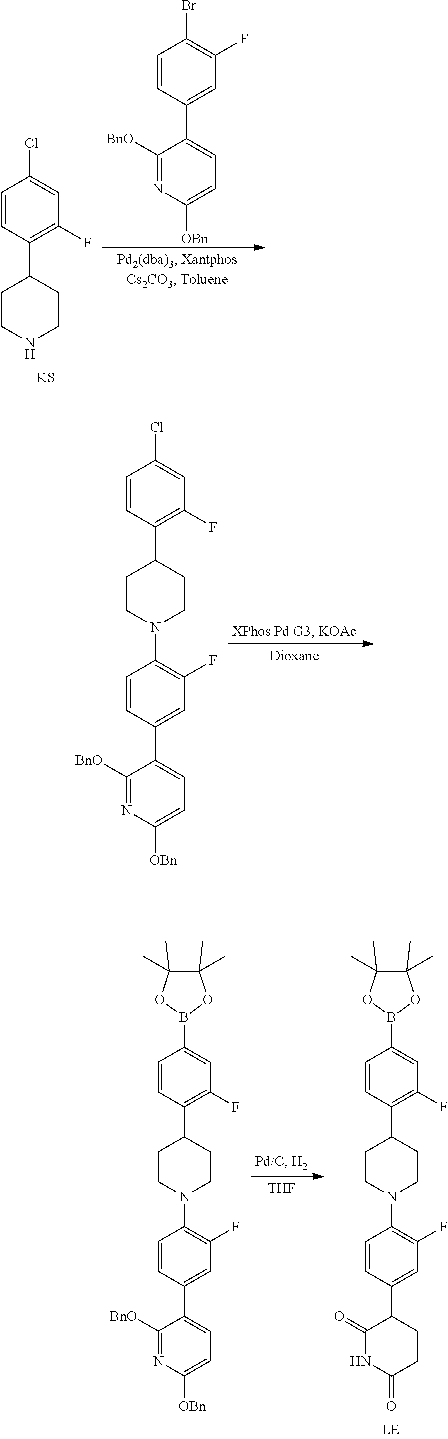

### Step 1—2,6-Bis(benzyloxy)-3-(4-(4-(4-chloro-2-fluorophenyl)piperidin-1-yl)-3-fluorophenyl)pyridine

A mixture of 4-(4-chloro-2-fluorophenyl)piperidine (160 mg, 748 μmol, Intermediate KS), 2,6-bis(benzyloxy)-3-(4-bromo-3-fluorophenyl)pyridine (347 mg, 748 μmol, CAS #2902652-28-0), Pd₂(dba)₃ (48.0 mg, 52.4 μmol), Xantphos (64.9 mg, 112 μmol) and Cs₂CO₃ (609 mg, 1.87 mmol) in toluene (10 mL) was degassed and purged with N₂ three times. Then the mixture was stirred at 100° C. for 6 hrs under N₂ atmosphere. On completion, the mixture was quenched with water (10 mL) and extracted with ethyl acetate (2×20 mL). The combined organic layers were washed with brine (20 mL) and dried over anhydrous sodium sulfate, filtered and the filtrate was concentrated in vacuo to give the crude residue. The residue was purified by reversed-phase HPLC (0.1% FA condition) to give the title compound (320 mg, 72% yield) as a white solid. LC-MS (ESI⁺) m/z 597.2 (M+H)⁺.

### Step 2—2,6-Bis(benzyloxy)-3-(3-fluoro-4-(4-(2-fluoro-4-(4,4,5,5-tetramethyl-1,3,2-dioxaborolan-2-yl)phenyl)piperidin-1-yl)phenyl)pyridine

A mixture of 2,6-bis(benzyloxy)-3-(4-(4-(4-chloro-2-fluorophenyl)piperidin-1-yl)-3-fluorophenyl)pyridine (320 mg, 535 μmol), 4,4,5,5-tetramethyl-2-(4,4,5,5-tetramethyl-1,3,2-dioxaborolan-2-yl)-1,3,2-dioxaborolane (204 mg, 803 μmol), XPhos Pd G3 (45.3 mg, 53.5 μmol), and AcOK (157 mg, 1.61 mmol) in dioxane (10 mL) was degassed and purged with N₂ three times. Then the mixture was stirred at 80° C. for 2 hrs under N₂ atmosphere. On completion, the mixture was quenched with water (20 mL) and extracted with ethyl acetate (2×20 mL). The combined organic layers were washed with brine (20 mL) and dried over anhydrous sodium sulfate, filtered and the filtrate was concentrated in vacuo to give the crude residue. The residue was purified by column chromatography (SiO₂, Petroleum ether:Ethyl acetate=1/0 to 10/1) to give the title compound (300 mg, 81% yield) as a white solid. LC-MS (ESI⁺) m/z 689.3 (M+H)⁺.

### Step 3—3-(3-Fluoro-4-(4-(2-fluoro-4-(4,4,5,5-tetramethyl-1,3,2-dioxaborolan-2-yl)phenyl)piperidin-1-yl)phenyl)piperidine-2,6-dione

To a solution of 2,6-bis(benzyloxy)-3-(3-fluoro-4-(4-(2-fluoro-4-(4,4,5,5-tetramethyl-1,3,2-dioxaborolan-2-yl)phenyl)piperidin-1-yl)phenyl)pyridine (300 mg, 435 μmol) in THF (10 mL) was added Pd/C (10 wt %, 300 mg) under N₂ atmosphere. The suspension was degassed and purged with H₂ three times. The mixture was then stirred under H₂ (15 psi) at 25° C. for 12 hrs. On completion, the mixture was filtered and the filtrate was concentrated in vacuo to give the crude residue. The residue was purified by column chromatography (SiO₂, Petroleum ether:Ethyl acetate=10/1 to 1/1) to give the title compound (200 mg, 90% yield) as a white solid. LC-MS (ESI⁺) m/z 511.1 (M+H)⁺.

### 3-[4-[2-[4-(4-Chloro-2,5-difluoro-phenyl)piperazin-1-yl]ethyl]-3-methyl-indazol-1-yl]piperidine-2,6-dione (Intermediate LF)

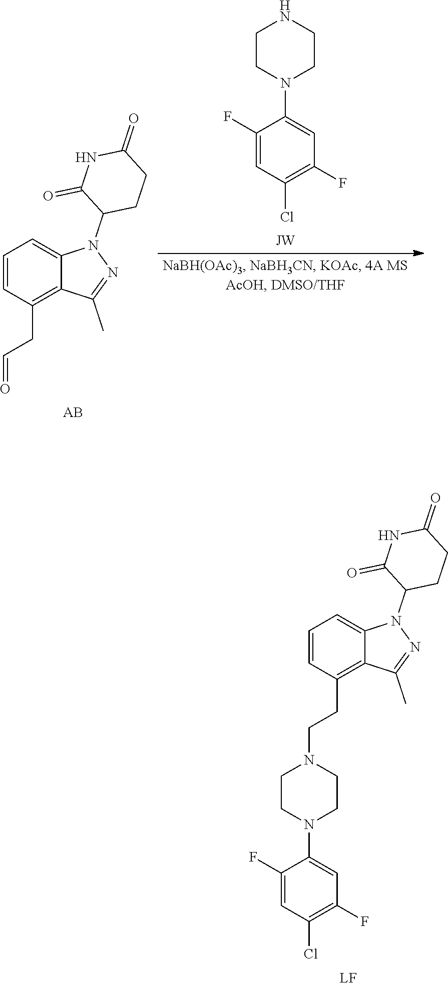 [SMILES: Cc1nn(C2CCC(=O)NC2=O)c2cccc(CC=O)c12 ; Fc1cc(N2CCNCC2)c(F)cc1Cl ; "AB" ; Cc1nn(C2CCC(=O)NC2=O)c2cccc(CCN3CCN(c4cc(F)c(Cl)cc4F)CC3)c12]

To a solution of 1-(4-chloro-2,5-difluoro-phenyl)piperazine (180 mg, 774 μmol, Intermediate JW) in DMSO (2 mL) and THF (2 mL) was added KOAc (75.9 mg, 774 gmol) and 4 Å molecular sieves (774 μmol), 2-[1-(2,6-dioxo-3-piperidyl)-3-methyl-indazol-4-yl]acetaldehyde (221 mg, 774 μmol, Intermediate AB), and AcOH (46.5 mg, 774 μmol, 44.3 μL) and the mixture was stirred for 1 hr at rt. Next, NaBH(OAc)₃ (492 mg, 2.32 mmol) and NaBH₃CN (146 mg, 2.32 mmol) was added at 0° C., then the mixture was stirred at 0-25° C. for 2 hrs. On completion, the reaction mixture was filtered and the filtrate was concentrated in vacuo to give the crude residue. The residue was purified by prep-HPLC (column: CD02-Waters XBridge BEH C18 150*25*10 um; mobile phase: [water (NH₄HCO₃)-ACN]; gradient:42%-72% B over 10 minutes) to give the title compound (300 mg, 70% yield) as a white solid. LC-MS (ESI⁺) m/z 502.2 (M+H)⁺.

### 6-Chloro-4-(3-(1-(2,6-dioxopiperidin-3-yl)-3-methyl-2-oxo-2,3-dihydro-1H-benzo[d]imidazol-4-yl)-3-azaspiro[5.5]undecan-9-yl)-7-fluoro-N,N-dimethyl-1H-indole-2-carboxamide (Intermediate LG)

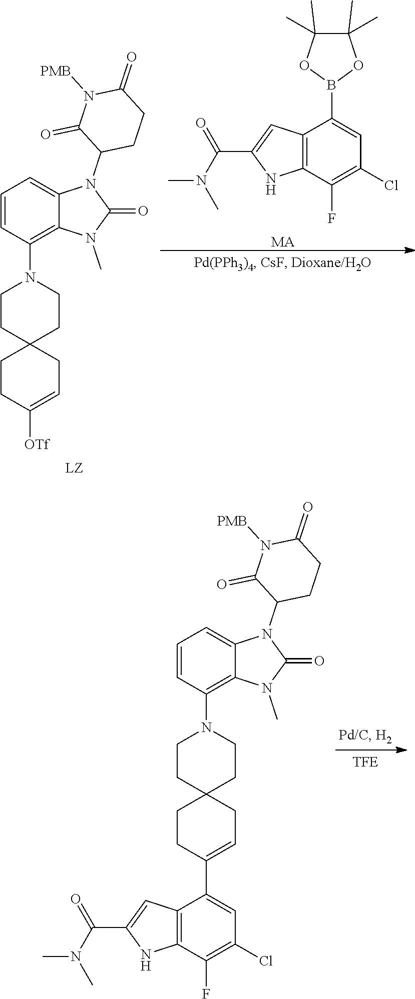

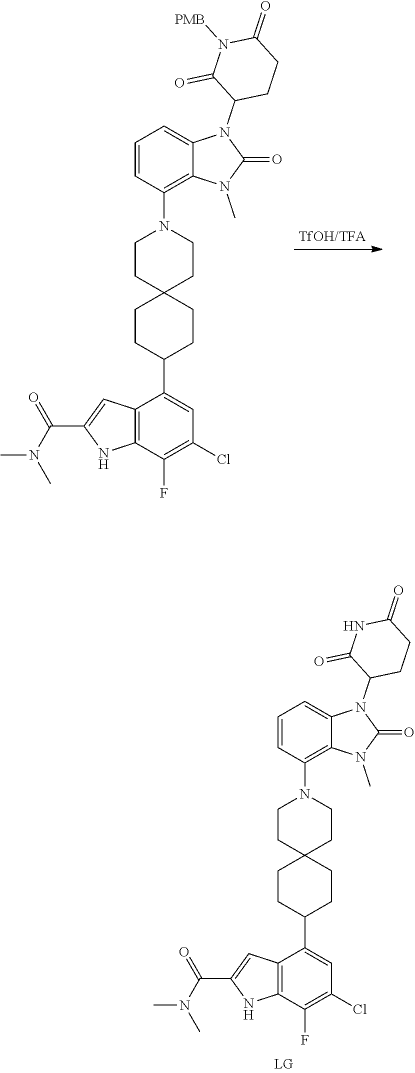

### Step 1—6-Chloro-7-fluoro-4-(3-(1-(1-(4-methoxybenzyl)-2,6-dioxopiperidin-3-yl)-3-methyl-2-oxo-2,3-dihydro-1H-benzo[d]imidazol-4-yl)-3-azaspiro[5.5]undec-8-en-9-yl)-N,N-dimethyl-1H-indole-2-carboxamide

A mixture of 3-(1-(1-(4-methoxybenzyl)-2,6-dioxopiperidin-3-yl)-3-methyl-2-oxo-2,3-dihydro-1H-benzo[d]imidazol-4-yl)-3-azaspiro[5.5]undec-8-en-9-yl trifluoromethanesulfonate (2.3 g, 3.4 mmol, Intermediate LZ), 6-chloro-7-fluoro-N,N-dimethyl-4-(4,4,5,5-tetramethyl-1,3,2-dioxaborolan-2-yl)-1H-indole-2-carboxamide (1.50 g, 4.08 mmol. Intermediate MA), Pd(PPh₃)₄ (589 mg, 509 μmol), and CsF (1.55 g, 10.2 mmol) in dioxane (30 mL) and H₂O (8 mL) was degassed and purged with N₂ three times. Then the mixture was stirred at 80° C. for 2 hrs under N₂ atmosphere. On completion, the reaction mixture was concentrated under reduced pressure to give a residue. The residue was purified by column chromatography (SiO₂, Petroleum ether/Ethyl acetate=3/1 to 1/5) to give the title compound (1.4 g, 50% yield) as a brown solid. LC-MS (ESI⁺) m/z 767.4 (M+H)⁺.

### Step 2—6-Chloro-7-fluoro-4-(3-(1-(1-(4-methoxybenzyl)-2,6-dioxopiperidin-3-yl)-3-methyl-2-oxo-2,3-dihydro-1H-benzo[d]imidazol-4-yl)-3-azaspiro[5.5]undecan-9-yl)-N,N-dimethyl-1H-indole-2-carboxamide

To a solution of 6-chloro-7-fluoro-4-(3-(1-(1-(4-methoxybenzyl)-2,6-dioxopiperidin-3-yl)-3-methyl-2-oxo-2,3-dihydro-1H-benzo[d]imidazol-4-yl)-3-azaspiro[5.5]undec-8-en-9-yl)-N,N-dimethyl-1H-indole-2-carboxamide (1.3 g, 1.69 mmol) in TFE (15 mL) was added Pd/C under N₂ atmosphere. The suspension was degassed and purged with H₂ three times. Then the mixture was stirred under H₂ (15 psi) at 25° C. for 12 hrs. On completion, the reaction mixture was filtered and the filtrate was concentrated in vacuo to give the crude residue. The crude residue was purified by reversed-phase HPLC (0.8 g/L ammonium bicarbonate) to give the title compound (600 mg, 57% yield) as a white solid. LC-MS (ESI⁺) m/z 769.3 (M+H)⁺.

### Step 3—6-Chloro-4-(3-(1-(2,6-dioxopiperidin-3-yl)-3-methyl-2-oxo-2,3-dihydro-1H-benzo[d]imidazol-4-yl)-3-azaspiro[5.5]undecan-9-yl)-7-fluoro-N,N-dimethyl-1H-indole-2-carboxamide

To a solution of 6-chloro-7-fluoro-4-(3-(1-(1-(4-methoxybenzyl)-2,6-dioxopiperidin-3-yl)-3-methyl-2-oxo-2,3-dihydro-1H-benzo[d]imidazol-4-yl)-3-azaspiro[5.5]undecan-9-yl)-N,N-dimethyl-1H-indole-2-carboxamide (250 mg, 324 μmol) in TFA (3 mL) was added TfOH (4.54 g, 30.2 mmol, 2.68 mL). The mixture was then stirred at 70° C. for 2 hrs. On completion, the reaction mixture was purified by reversed-phase HPLC (0.8 g/L ammonium bicarbonate) to give the title compound (120 mg, 57% yield) as a white solid. LC-MS (ESI⁺) m/z 649.3 (M+H)⁺.

### 4-Bromo-5-fluoro-3-methyl-1H-indazole (Intermediate LH)

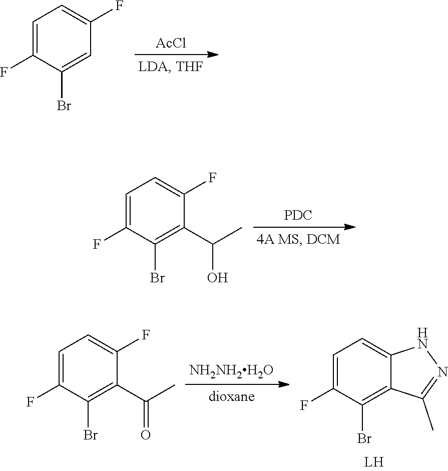

### Step 1—1-(2-Bromo-3,6-difluorophenyl)ethanol

To a solution of 2-bromo-1,4-difluorobenzene (10 g, 51.8 mmol, CAS #399-94-0) in THF (100 mL) was added LDA (2 M, 31.0 mL) at −78° C., and the reaction was stirred at −78° C. for 1 hr. Then acetaldehyde (5 M, 15.5 mL) was added into the mixture and the reaction was stirred at 25° C. for 1 hr. On completion, the reaction mixture was quenched with NH4Cl (40 mL) and extracted with ethyl acetate (3×50 mL). The combined organic layers were washed with brine (200 mL) and dried over anhydrous sodium sulfate, filtered and the filtrate was concentrated in vacuo to give the crude residue. The residue was purified by column chromatography (SiO₂, Petroleum ether:Ethyl acetate=1/0 to 10/1) to give the title compound (10 g, 73% yield) as a yellow oil. H NMR (400 MHz, DMSO-d₆) 5=7.38-7.24 (m, 2H), 5.50 (d, J=4.4 Hz, 1H), 5.22 (q, J=6.0 Hz, 1H), 1.44 (d, J=6.8 Hz, 3H).

### Step 2—1-(2-Bromo-3,6-difluorophenyl)ethanone

To a solution of 1-(2-bromo-3,6-difluorophenyl)ethanol (10 g, 42 mmol) in DCM (100 mL) was added 4 Å molecular sieves (10 g) and PDC (47.6 g, 126 mmol). The reaction was then stirred at 25° C. for 12 hrs. On completion, the reaction mixture was filtered and the filtrate was concentrated in vacuo to give the crude residue. The residue was purified by column chromatography (SiO₂, Petroleum ether:Ethyl acetate=1/0 to 10/1) to give the title compound (8.3 g, 75% yield) as a yellow oil. ¹H NMR (400 MHz, DMSO-d₆) δ=7.60-7.53 (m, 1H), 7.52-7.45 (m, 1H), 2.58 (s, 3H).

### Step 3—4-Bromo-5-fluoro-3-methyl-1H-indazole

To a solution of 1-(2-bromo-3,6-difluorophenyl)ethanone (7.6 g, 32 mmol) in dioxane (80 mL) was added N₂H₄·H₂O (5.71 g, 97.0 mmol, 5.54 mL, 85% solution) at 25° C. under N₂ atmosphere. The reaction was then stirred at 90° C. for 24 hrs under N₂ atmosphere. On completion, the reaction mixture was concentrated in vacuo to give the crude residue. The crude residue was purified by reversed-phase HPLC (0.8 g/L ammonium bicarbonate) to give the title compound (700 mg, 8% yield) as a white solid. LC-MS (ESI⁺) m/z 229.0 (M+H)⁺.

### 3-(4-Bromo-5-fluoro-3-methyl-1H-indazol-1-yl)piperidine-2,6-dione (Intermediate LI)

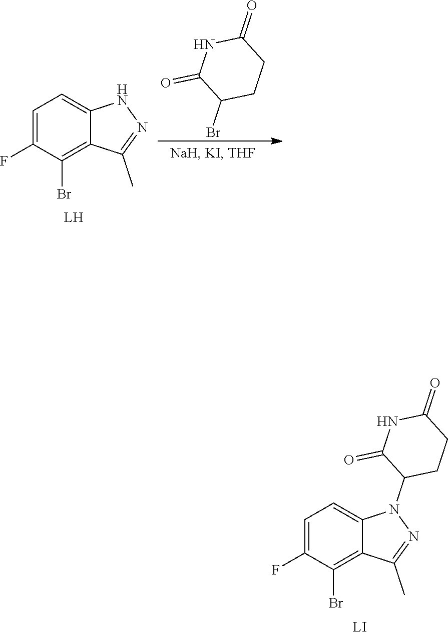 [SMILES: Cc1n[nH]c2ccc(F)c(Br)c12 ; O=C1CCC(Br)C(=O)N1 ; Cc1nn(C2CCC(=O)NC2=O)c2ccc(F)c(Br)c12]

To a solution of 4-bromo-5-fluoro-3-methyl-1H-indazole (600 mg, 2.62 mmol, Intermediate LH) in THF (10 mL) was added NaH (314 mg, 7.86 mmol, 60% dispersion in mineral oil) at 0° C. The reaction was then stirred at 0° C. for 2 hrs under N₂ atmosphere. Next, 3-bromopiperidine-2,6-dione (754 mg, 3.93 mmol, CAS #62595-74-8) and KI (347 mg, 2.10 mmol) was added into the mixture. Then the reaction was stirred at 25° C. for 2 hrs under N₂ atmosphere. On completion, the reaction mixture was quenched with NH₄Cl (10 mL) and extracted with ethyl acetate (3×30 mL). The combined organic layers were washed with brine (50 mL) and dried over anhydrous sodium sulfate, filtered and the filtrate was concentrated in vacuo to give the title compound (750 mg) as a brown solid. LC-MS (ESI⁺) m/z 340.0 (M+H)⁺.

### 2-(1-(2,6-Dioxopiperidin-3-yl)-5-fluoro-3-methyl-1H-indazol-4-yl)acetaldehyde (Intermediate LJ)

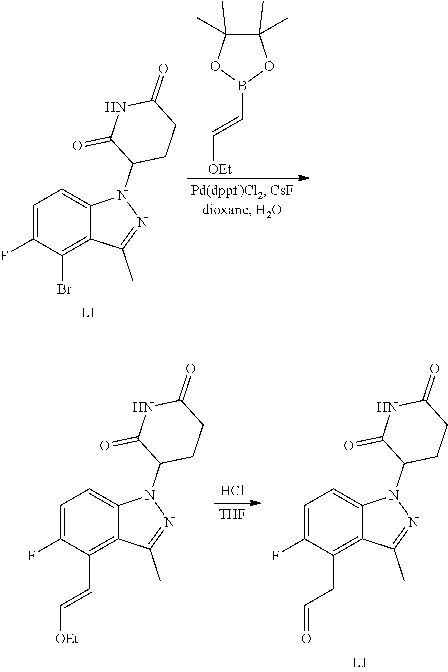

### Step 1—(E)-3-(4-(2-ethoxyvinyl)-5-fluoro-3-methyl-1H-indazol-1-yl)piperidine-2,6-dione

To a solution of 3-(4-bromo-5-fluoro-3-methyl-1H-indazol-1-yl)piperidine-2,6-dione (650 mg, 1.91 mmol, Intermediate LI) in dioxane (8 mL) and H₂O (2 mL) was added (E)-2-(2-ethoxyvinyl)-4,4,5,5-tetramethyl-1,3,2-dioxaborolane (378 mg, 1.91 mmol, CAS #1201905-61-4), CsF (870 mg, 5.73 mmol) and Pd(dppf)Cl₂·CH₂Cl₂ (156 mg, 191 μmol). The reaction was then stirred at 90° C. for 1 hr. On completion, the reaction mixture was concentrated in vacuo to give the crude residue. The crude residue was purified by reversed-phase HPLC (0.8 g/L ammonium bicarbonate) to give the title compound (350 mg, 52% yield) as a brown solid. LC-MS (ESI⁺) m/z 332.1 (M+H)⁺.

### Step 2—2-(1-(2,6-Dioxopiperidin-3-yl)-5-fluoro-3-methyl-1H-indazol-4-yl)acetaldehyde

To a solution of (E)-3-(4-(2-ethoxyvinyl)-5-fluoro-3-methyl-1H-indazol-1-yl)piperidine-2,6-dione (300 mg, 905 μmol) in THF (5 mL) was added HCl (6 M, 5 mL). The reaction was then stirred at 25° C. for 0.5 hrs. On completion, the reaction mixture was concentrated in vacuo to give the crude residue. The crude residue was purified by reversed-phase HPLC (0.1% FA condition) to give the title compound (60 mg, 21% yield) as a white solid. LC-MS (ESI⁺) m/z 303.9 (M+H)⁺.

### (R)-6-(1-(3-(1H-pyrazol-1-yl)propanoyl)piperidin-3-yl)-4-(2-chloro-5-fluoro-4-(piperazin-1-yl)phenyl)-7-fluoro-N,N-dimethyl-1H-indole-2-carboxamide (Intermediate LK)

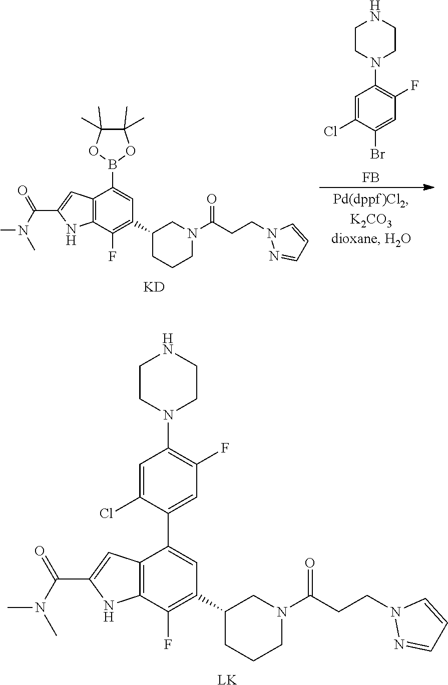 [SMILES: CN(C)C(=O)c1cc2c(B3OC(C)(C)C(C)(C)O3)cc([C@H]3CCCN(C(=O)CCn4cccn4)C3)c(F)c2[nH]1 ; Fc1cc(Br)c(Cl)cc1N1CCNCC1 ; "FB" ; "KD" ; CN(C)C(=O)c1cc2c(-c3cc(F)c(N4CCNCC4)cc3Cl)cc([C@H]3CCCN(C(=O)CCn4cccn4)C3)c(F)c2[nH]1 ; "LK"]

To a solution of (R)-6-(1-(3-(1H-pyrazol-1-yl)propanoyl)piperidin-3-yl)-7-fluoro-N,N-dimethyl-4-(4,4,5,5-tetramethyl-1,3,2-dioxaborolan-2-yl)-1H-indole-2-carboxamide (90 mg, 167 μmol, Intermediate KD) in dioxane (2 mL) and H₂O (0.5 mL) was added 1-(4-bromo-5-chloro-2-fluorophenyl)piperazine (58.9 mg, 200 μmol, Intermediate FB), K₂CO₃ (69.4 mg, 502 μmol) and Pd(dppf)Cl₂CH₂Cl₂ (13.6 mg, 16.7 μmol). The reaction was then stirred at 80° C. for 1 hr. On completion, the reaction mixture was concentrated in vacuo to give the crude residue. The residue was purified by column chromatography (SiO₂, Dichloromethane:Methanol=20/1 to 10/1) to give the title compound (110 mg, 90% yield) as a black solid. LC-MS (ESI⁺) m/z 624.4 (M+H)⁺.

### 4-(4-Chloro-2,3-difluorophenyl)piperidine (Intermediate LL)

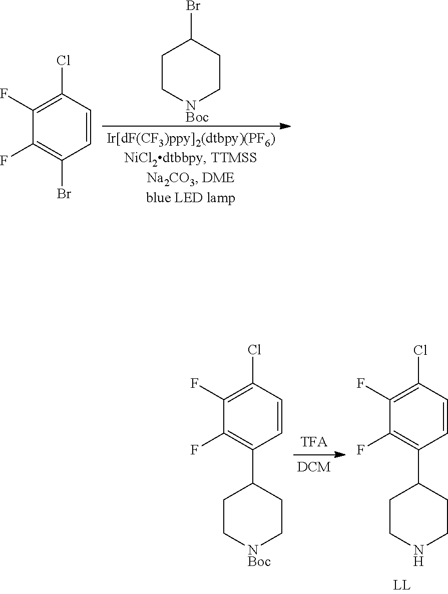 [SMILES: Fc1c(Cl)ccc(Br)c1F ; CC(C)(C)OC(=O)N1CCC(Br)CC1 ; CC(C)(C)OC(=O)N1CCC(c2ccc(Cl)c(F)c2F)CC1 ; Fc1c(Cl)ccc(C2CCNCC2)c1F]

### Step 1—Tert-butyl 4-(4-chloro-2,3-difluorophenyl)piperidine-1-carboxylate

A mixture of 1-bromo-4-chloro-2,3-difluorobenzene (10 g, 44 mmol, CAS #1000574-47-9), tert-butyl 4-bromopiperidine-1-carboxylate (11.61 g, 44.0 mmol, CAS #180695-79-8), Na₂CO₃ (4.66 g, 44.0 mmol) in DME (300 mL) was degassed and purged with N₂ three times. Then the mixture was stirred at 25° C. for 12 hrs under N₂ atmosphere. On completion, the reaction mixture was quenched with water (300 mL) and extracted with EA (3×300 mL). The combined organic layers were washed with aqueous NaCl (300 mL) and dried over anhydrous sodium sulfate, filtered and the filtrate was concentrated in vacuo to give the crude residue. The residue was purified by column chromatography (SiO₂, Petroleum ether:Ethyl acetate=1/0) to give the title compound (10 g) as a yellow solid. LC-MS (ESI⁺) m/z 276.0 (M−55)⁺.

### Step 2—4-(4-Chloro-2,3-difluorophenyl)piperidine

To a solution of tert-butyl 4-(4-chloro-2,3-difluorophenyl)piperidine-1-carboxylate (500 mg, 1.51 mmol) in DCM (5 mL) was added TFA (1.54 g, 13.5 mmol. 1.00 mL). The mixture was then stirred at 25° C. for 12 hrs. On completion, the reaction mixture was concentrated in vacuo to give the crude residue. The crude residue was purified by reversed-phase HPLC (0.8 g/L ammonium bicarbonate) to give the title compound (300 mg, 72% yield, FA) as a white solid. LC-MS (ESI⁺) m/z 232.0 (M+H)⁺.

### 2,6-Bis(benzyloxy)-3-(4-(4-(4-chloro-2,3-difluorophenyl)piperidin-1-yl)-3-fluorophenyl)pyridine (Intermediate LM)

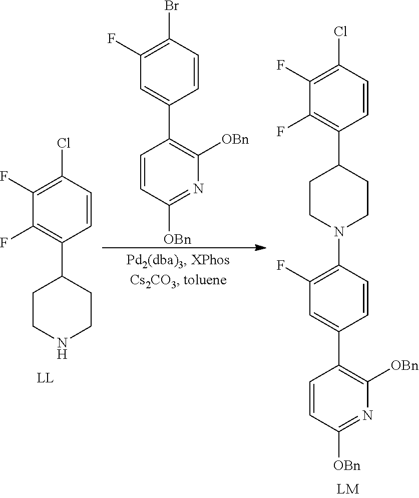 [SMILES: Fc1c(Cl)ccc(C2CCNCC2)c1F ; Fc1cc(-c2ccc(OCc3ccccc3)nc2OCc2ccccc2)ccc1Br ; Fc1cc(-c2ccc(OCc3ccccc3)nc2OCc2ccccc2)ccc1N1CCC(c2ccc(Cl)c(F)c2F)CC1]

A mixture of 4-(4-chloro-2,3-difluorophenyl)piperidine (300 mg, 1.08 mmol, FA, Intermediate LL). 2,6-bis(benzyloxy)-3-(4-bromo-3-fluorophenyl)pyridine (600 mg, 1.29 mmol, CAS #2902652-28-0), XPhos (61.8 mg, 130 μmol), Pd₂(dba)₃ (49.5 mg, 54.0 μmol) and Cs₂CO₃ (1.41 g, 4.32 mmol) in toluene (6 mL) was degassed and purged with N₂ three times. Then the mixture was stirred at 100° C. for 2 hrs under N₂ atmosphere. On completion, the reaction mixture was quenched with water (30 mL) and extracted with dichloromethane (3×30 mL). The combined organic layers were washed with aqueous NaCl (30 mL) and dried over anhydrous sodium sulfate, filtered and the filtrate was concentrated in vacuo to give the crude residue. The crude residue was purified by reversed-phase HPLC (0.1% FA condition) to give the title compound (500 mg, 75% yield) as a yellow solid. LC-MS (ESI⁺) m/z 615.2 (M+H)⁺.

### 3-(4-(4-(2,3-Difluoro-4-(4,4,5,5-tetramethyl-1,3,2-dioxaborolan-2-yl)phenyl)piperidin-1-yl)-3-fluorophenyl)piperidine-2,6-dione (Intermediate LN)

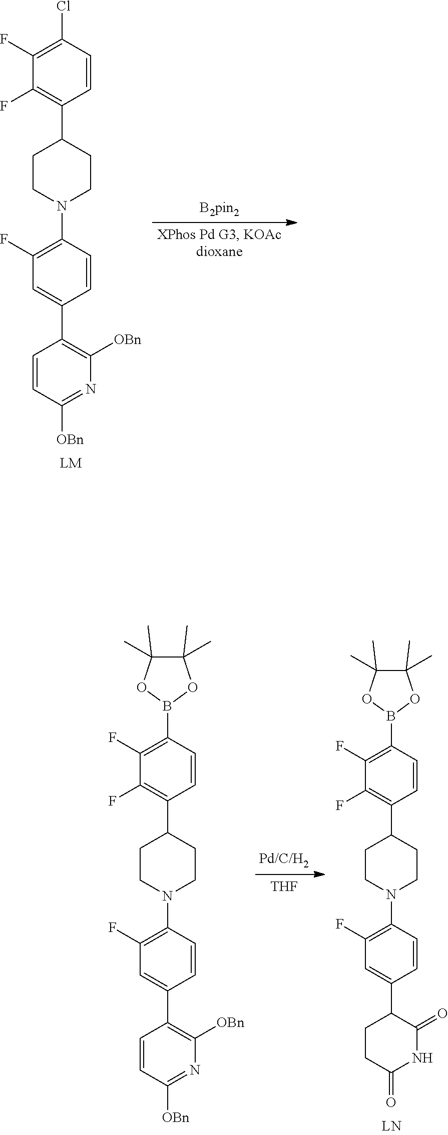 [SMILES: Fc1cc(-c2ccc(OCc3ccccc3)nc2OCc2ccccc2)ccc1N1CCC(c2ccc(Cl)c(F)c2F)CC1 ; CC1(C)OB(c2ccc(C3CCN(c4ccc(-c5ccc(OCc6ccccc6)nc5OCc5ccccc5)cc4F)CC3)c(F)c2F)OC1(C)C ; CC1(C)OB(c2ccc(C3CCN(c4ccc(C5CCC(=O)NC5=O)cc4F)CC3)c(F)c2F)OC1(C)C ; "THF"]

### Step 1—2,6-Bis(benzyloxy)-3-(4-(4-(2,3-difluoro-4-(4,4,5,5-tetramethyl-1,3,2-dioxaborolan-2-yl)phenyl)piperidin-1-yl)-3-fluorophenyl)pyridine

A mixture of 2,6-bis(benzyloxy)-3-(4-(4-(4-chloro-2,3-difluorophenyl)piperidin-1-yl)-3-fluorophenyl)pyridine (500 mg, 813 μmol, Intermediate LM), 4,4,4′,4′,5,5,5′,5′-octamethyl-2,2′-bi(1,3,2-dioxaborolane) (619 mg, 2.44 mmol, CAS #73183-34-3), AcOK (239 mg, 2.44 mmol), and XPhos Pd G3 (68.8 mg, 81.3 μmol) in dioxane (5 mL) was degassed and purged with N₂ three times. Then the mixture was stirred at 80° C. for 2 hrs under N₂ atmosphere. On completion, the reaction mixture was quenched with water (30 mL) and extracted with dichloromethane (3×30 mL). The combined organic layers were washed with aqueous NaCl (30 mL) and dried over anhydrous sodium sulfate, filtered and the filtrate was concentrated in vacuo to give the crude residue. The residue was purified by column chromatography (SiO₂, Dichloromethane:Methanol=10/1) to give the title compound (350 mg, 61% yield) as a yellow oil solid. LC-MS (ESI⁺) m/z 707.1 (M+H)⁺.

### Step 2—3-(4-(4-(2,3-Difluoro-4-(4,4,5,5-tetramethyl-1,3,2-dioxaborolan-2-yl)phenyl)piperidin-1-yl)-3-fluorophenyl)piperidine-2,6-dione

A mixture of 2,6-bis(benzyloxy)-3-(4-(4-(2,3-difluoro-4-(4,4,5,5-tetramethyl-1,3,2-dioxaborolan-2-yl)phenyl)piperidin-1-yl)-3-fluorophenyl)pyridine (350 mg, 495 μmol), Pd/C (350 mg, 10 wt %) in THF (5 mL) was degassed and purged with H₂ three times. Then the mixture was stirred at 25° C. for 12 hrs under N₂ atmosphere. On completion, the reaction was filtered through kieselguhr carefully, and the filtrate was concentrated in vacuo to give a residue. The residue was purified by column chromatography (SiO₂, Dichloromethan:Methanol=10/1) to give the title compound (100 mg, 38% yield) as a green solid. LC-MS (ESI⁺) m/z 528.1 (M+H)⁺.

### 3-(4-(2-(4-(4-Bromo-5-chloro-2-fluorophenyl)piperazin-1-yl)ethyl)-3-methyl-1H-indazol-1-yl)piperidine-2,6-dione (Intermediate LO)

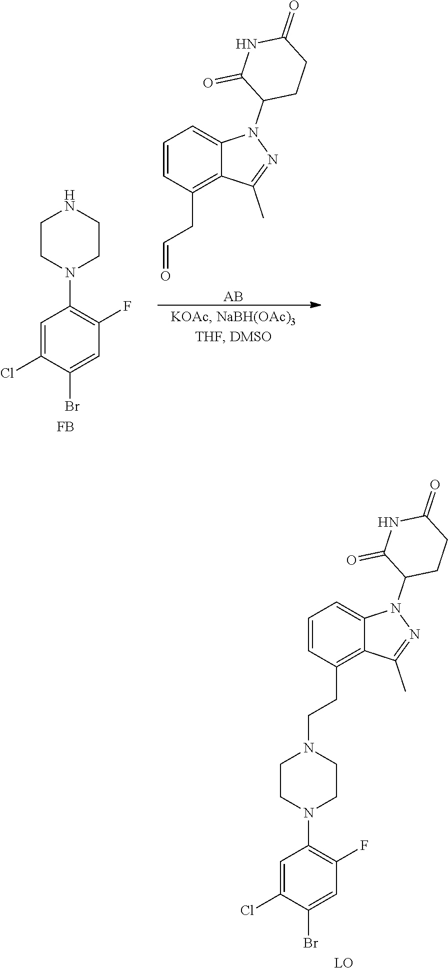

A solution of 1-(4-bromo-5-chloro-2-fluorophenyl)piperazine (400 mg, 1.36 mmol, Intermediate FB), 2-(1-(2,6-dioxopiperidin-3-yl)-3-methyl-1H-indazol-4-yl)acetaldehyde (505 mg, 1.77 mmol, Intermediate AB), KOAc (267 mg, 2.73 mmol), and NaBH(OAc)₃ (866 mg, 4.09 mmol) in DMSO (0.2 mL) and THF (0.2 mL) was stirred at 25° C. for 2 hrs. On completion, the reaction mixture was quenched with water (20 mL) and extracted with ethyl acetate (3×20 mL). The combined organic layers were washed with brine (25 mL) and dried over anhydrous sodium sulfate, filtered and the filtrate was concentrated in vacuo to give the crude residue. The residue was purified by purified by reversed-phase HPLC (0.1% FA condition) to give the title compound (100 mg) as a brown solid. LC-MS (ESI⁺) m/z 562.0 (M+H)⁺.

### (R)-methyl 4-chloro-7-fluoro-6-(piperidin-3-yl)-1H-indole-2-carboxylate (Intermediate LP)

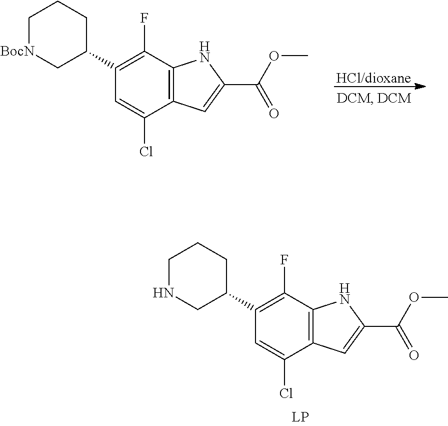

To a solution of (R)-methyl 6-(1-(tert-butoxycarbonyl)piperidin-3-yl)-4-chloro-7-fluoro-1H-indole-2-carboxylate (2 g, 4.87 mmol, synthesized via Step 1 of Intermediate DC) in anhydrous DCM (30 mL) was added HCl/dioxane (8 M, 6 mL) at 25° C. under nitrogen atmosphere, then the reaction was stirred at 25° C. for 2 h under nitrogen atmosphere. On completion, the mixture was concentrated to give the title compound (2 g, HCl) as a white solid. LC-MS (ESI⁺) m/z 311.0 (M+H)⁺.

### (R)-6-(1-(3-(1H-pyrazol-1-yl)propanoyl)piperidin-3-yl)-4-chloro-7-fluoro-1H-indole-2-carboxylic acid (Intermediate LQ)

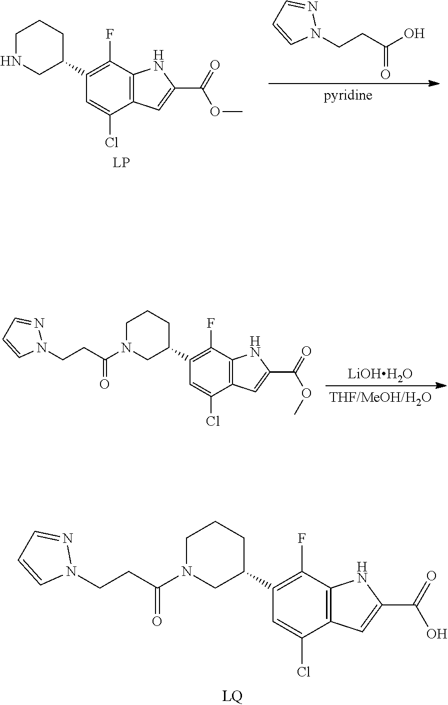

### Step 1—Methyl (R)-6-(1-(3-(1H-pyrazol-1-yl)propanoyl)piperidin-3-yl)-4-chloro-7-fluoro-1H-indole-2-carboxylate

To a solution of methyl (R)-4-chloro-7-fluoro-6-(piperidin-3-yl)-1H-indole-2-carboxylate (800 mg, 2.30 mmol, HCl, Intermediate LP), 3-(1H-pyrazol-1-yl)propanoic acid (366 mg, 2.07 mmol, HCl, CAS #89532-73-0) in pyridine (5 mL) was added EDCI (663 mg, 3.46 mmol). The mixture was then stirred at 25° C. for 2 hrs. On completion, the reaction mixture was quenched with water (5 mL) and extracted with EA (3×10 mL). The combined organic layers were washed with aqueous NaCl (30 mL) and dried over anhydrous sodium sulfate, filtered and the filtrate was concentrated in vacuo to give the title compound (1.3 g) as yellow oil. LC-MS (ESI⁺) m/z 433.0 (M+H)⁺.

### Step 2—(R)-6-(1-(3-(1H-pyrazol-1-yl)propanoyl)piperidin-3-yl)-4-chloro-7-fluoro-1H-indole-2-carboxylic acid

To a solution of methyl (R)-6-(1-(3-(1H-pyrazol-1-yl)propanoyl)piperidin-3-yl)-4-chloro-7-fluoro-1H-indole-2-carboxylate (1.2 g, 2.8 mmol) in THF (8 mL), MeOH (2 mL), and H₂O (2 mL) was added LiOH·H₂O (582 mg, 13.9 mmol). Then the mixture was stirred at 25° C. for 12 hrs. On completion, the reaction mixture was quenched with water (30 mL) and extracted with EA (3×30 mL). The combined organic layers were washed with aqueous NaCl (30 mL) and dried over anhydrous sodium sulfate, filtered and the filtrate was concentrated in vacuo to give the title compound (1.2 g) as yellow oil. LC-MS (ESI⁺) m/z 419.2 (M+H)⁺.

### (R)-6-(1-(3-(1H-pyrazol-1-yl)propanoyl)piperidin-3-yl)-4-chloro-7-fluoro-N,N-dimethyl-1H-indole-2-carboxamide (Intermediate LR)

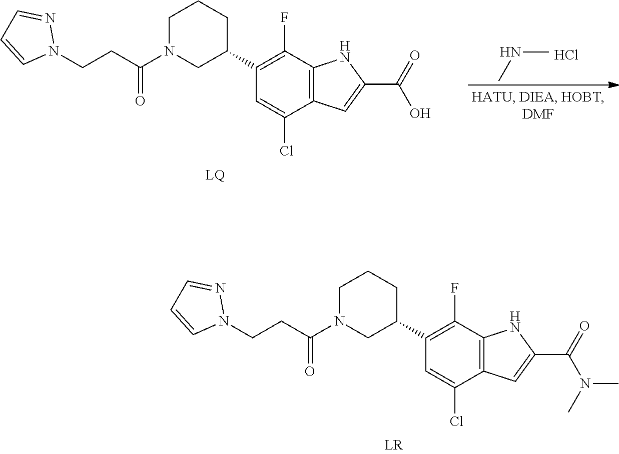

To a solution of (R)-6-(1-(3-(1H-pyrazol-1-yl)propanoyl)piperidin-3-yl)-4-chloro-7-fluoro-1H-indole-2-carboxylic acid (900 mg, 2.2 mmol, Intermediate LQ) in DMF (8 mL) was added HATU (1.06 g, 2.79 mmol), DIEA (2.22 g, 17.2 mmol, 2.99 mL), HOBt (581 mg, 4.30 mmol), and dimethylamine hydrochloride (175 mg, 2.15 mmol). The mixture was then stirred at 25° C. for 2 hrs. On completion, the reaction mixture was quenched with water (10 mL) and extracted with EA (3×20 mL). The combined organic layers were washed with aqueous NaCl (30 mL) and dried over anhydrous sodium sulfate, filtered and the filtrate was concentrated in vacuo to give the crude residue. The crude residue was purified by reversed-phase HPLC (0.1% FA condition) to give the title compound (680 mg, 71% yield) as a white solid. LC-MS (ESI⁺) m/z 446.0 (M+H)⁺.

### 2,6-Bis(benzyloxy)-3-(4-bromo-3,5-difluorophenyl)pyridine (Intermediate LS)

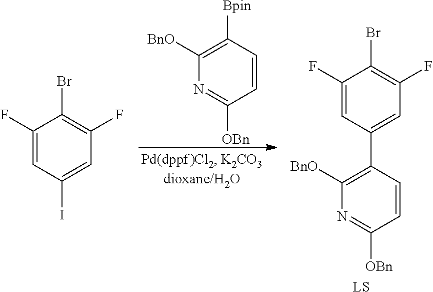

A mixture of 2-bromo-1,3-difluoro-5-iodo-benzene (5 g, 16 mmol, CAS #155906-10-8) 2,6-bis(benzyloxy)-3-(4,4,5,5-tetramethyl-1,3,2-dioxaborolan-2-yl)pyridine (6.54 g, 15.7 mmol, CAS #155906-10-8), K₂CO₃ (6.50 g, 47.0 mmol), and Pd(dppf)Cl₂·CH₂Cl₂ (640 mg, 784 μmol) in dioxane (80 mL) and H₂O (16 mL) was degassed and purged with N₂ three times. Then the mixture was stirred at 70° C. for 5 h under N₂ atmosphere. On completion, the reaction mixture was concentrated under reduced pressure. The mixture was purified by column chromatography (SiO₂, PE:EA=1:0) to give the title compound (6.4 g, 85% yield) as a yellow oil. LC-MS (ESI⁺) m/z 483.9 (M+H)⁺; ¹H NMR (400 MHz, DMSO-d6) δ ppm 5.41 (d, J=14.4 Hz, 4H), 6.58 (d, J=8.0 Hz. 1H), 7.25-7.45 (m, 10H), 7.49 (d, J=8.4 Hz. 2H), 7.88 (d, J=8.0 Hz, 1H).

### 3-(3,5-Difluoro-4-(4-(4-(4,4,5,5-tetramethyl-1,3,2-dioxaborolan-2-yl)phenyl)piperidin-1-yl)phenyl)piperidine-2,6-dione (Intermediate LT)

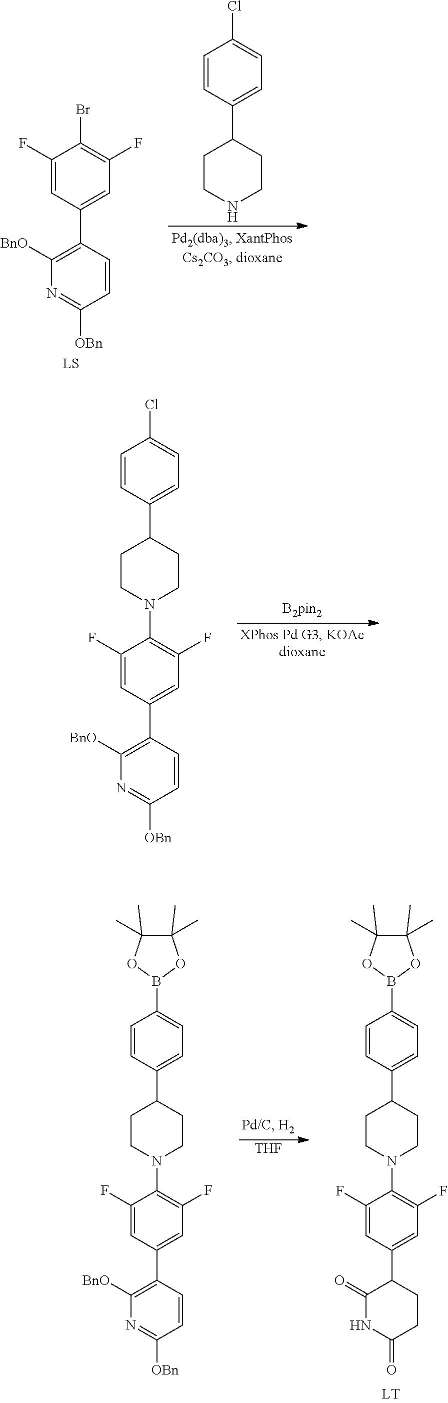

### Step 1—2,6-Bis(benzyloxy)-3-(4-(4-(4-chlorophenyl)piperidin-1-yl)-3,5-difluorophenyl)pyridine

A mixture of 2,6-bis(benzyloxy)-3-(4-bromo-3,5-difluorophenyl)pyridine (2.50 g, 5.18 mmol, Intermediate LS), 4-(4-chlorophenyl)piperidine (1.01 g, 5.18 mmol, CAS #26905-02-2), Cs₂CO₃ (4.22 g, 13.0 mmol), Xantphos (450 mg, 778 μmol) and Pd₂(dba)₃ (332 mg, 363 μmol) in toluene (40 mL) was degassed and purged with N₂ three times. Then the mixture was stirred at 100° C. for 12 h under N₂ atmosphere. On completion, the reaction mixture was filtered and concentrated under reduced pressure to give a residue. The crude product was purified by column chromatography (SiO₂, Petroleum ether/Ethyl acetate=0/1 to DCM:MeOH=4/1) to give the title compound (1.80 g, 58% yield) as a yellow solid. LC-MS (ESI⁺) m/z 597.4 (M+H)⁺: ¹H NMR (400 MHz, DMSO-d₆) δ=1.68-1.87 (m, 4H), 2.64-2.78 (m, 1H), 3.11-3.26 (m, 4H), 5.35-5.50 (m, 4H), 6.56 (d, J=8.4 Hz, 1H), 7.25-7.47 (m, 16H), 7.84 (d, J=8.2 Hz, 1H).

### Step 2—2,6-Bis(benzyloxy)-3-(3,5-difluoro-4-(4-(4-(4,4,5,5-tetramethyl-1,3,2-dioxaborolan-2-yl)phenyl)piperidin-1-yl)phenyl)pyridine

A mixture of 2,6-bis(benzyloxy)-3-(3,5-difluoro-4-(4-(4-(4,4,5,5-tetramethyl-1,3,2-dioxaborolan-2-yl)phenyl)piperidin-1-yl)phenyl)pyridine (1 g, 2 mmol), 4,4,5,5-tetramethyl-2-(4,4,5,5-tetramethyl-1,3,2-dioxaborolan-2-yl)-1,3,2-dioxaborolane (851 mg, 3.35 mmol), XPhos Pd G3 (213 mg, 251 μmol), and KOAc (493 mg, 5.02 mmol) in dioxane (10 mL) was degassed and purged with N₂ three times. Then the mixture was stirred at 80° C. for 12 h under N₂ atmosphere. On completion, the mixture was purified by column chromatography (SiO₂, Petroleum ether/Ethyl acetate=20/1 to DCM:MeOH=1:0) to give the title compound (521 mg, 45% yield) as a white solid. LC-MS (ESI⁺) m/z 689.3 (M+H)⁺.

### Step 3—3-(3,5-Difluoro-4-(4-(4-(4,4,5,5-tetramethyl-1,3,2-dioxaborolan-2-yl)phenyl)piperidin-1-yl)phenyl)piperidine-2,6-dione

A mixture of 2,6-bis(benzyloxy)-3-(3,5-difluoro-4-(4-(4-(4,4,5,5-tetramethyl-1,3,2-dioxaborolan-2-yl)phenyl)piperidin-1-yl)phenyl)pyridine (300 mg, 436 μmol) and Pd/C (3.00 g, 2.82 mmol, 10 wt %) in THF (3 mL) was degassed and purged with H₂ three times. Then the mixture was stirred at 30° C. for 12 h under H₂ atmosphere. On completion, the reaction mixture was filtered and concentrated under reduced pressure to give the title compound (180 mg) as a white solid. LC-MS (ESI⁺) m, z 511.2 (M+H)⁺.

### 3-(4-(2-(4-(4-Bromo-2,3-difluorophenyl)piperazin-1-yl)ethyl)-3-methyl-1H-indazol-1-yl)piperidine-2,6-dione (Intermediate LU)

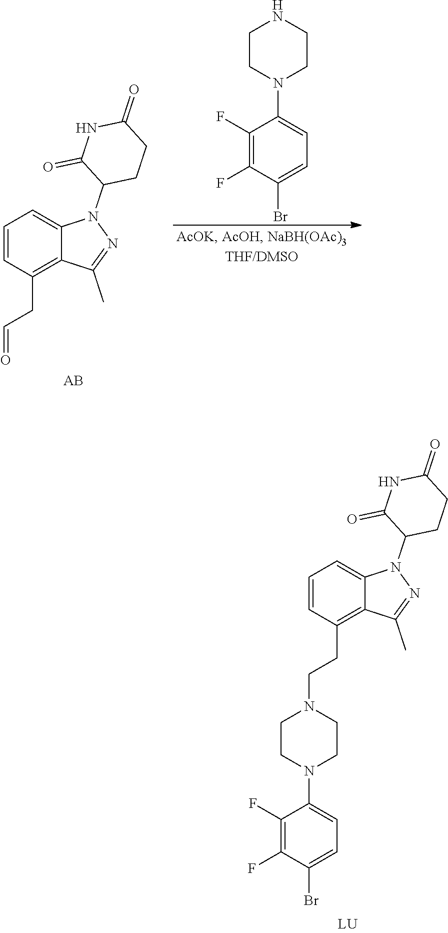 [SMILES: Cc1nn(C2CCC(=O)NC2=O)c2cccc(CC=O)c12 ; Fc1c(Br)ccc(N2CCNCC2)c1F ; "AB" ; Cc1nn(C2CCC(=O)NC2=O)c2cccc(CCN3CCN(c4ccc(Br)c(F)c4F)CC3)c12]

To a solution of 1-(4-bromo-2,3-difluoro-phenyl)piperazine (198 mg, 631 μmol) in THF (5 mL) and DMSO (0.5 mL) was added AcOK (186 mg, 1.89 mmol) at 25° C., and the mixture was stirred at 25° C. for 0.5 hr. Then 2-(1-(2,6-dioxopiperidin-3-yl)-3-methyl-1H-indazol-4-yl)acetaldehyde (180 mg, 631 μmol, Intermediate AB) and AcOH (303 mg, 5.05 mmol) was added at 25° C., and the reaction was stirred at 25° C. for 1 hr. Then NaBH(OAc)₃ (267 mg, 1.26 mmol) was added at 0° C., and the reaction was stirred at 25° C. for 1 hr. On completion, the reaction mixture was filtered and the filtrate was concentrated in vacuo to give the crude residue. The crude residue was purified by reversed-phase HPLC (column: CD07-Daisogel SP-100-8-ODS-PK 150×25×10 um; mobile phase: [water (NH₄HCO₃)-ACN]: gradient: 45%-65% B over 10 min) to give the title compound (245 mg, 50% yield) as a white solid. LC-MS (ESI⁺) m/z 547.9 (M+H)⁺.

### 3-(3,5-Difluoro-4-(4-(2-fluoro-4-(4,4,5,5-tetramethyl-1,3,2-dioxaborolan-2-yl)phenyl)piperidin-1-yl)phenyl)piperidine-2,6-dione (Intermediate LV)

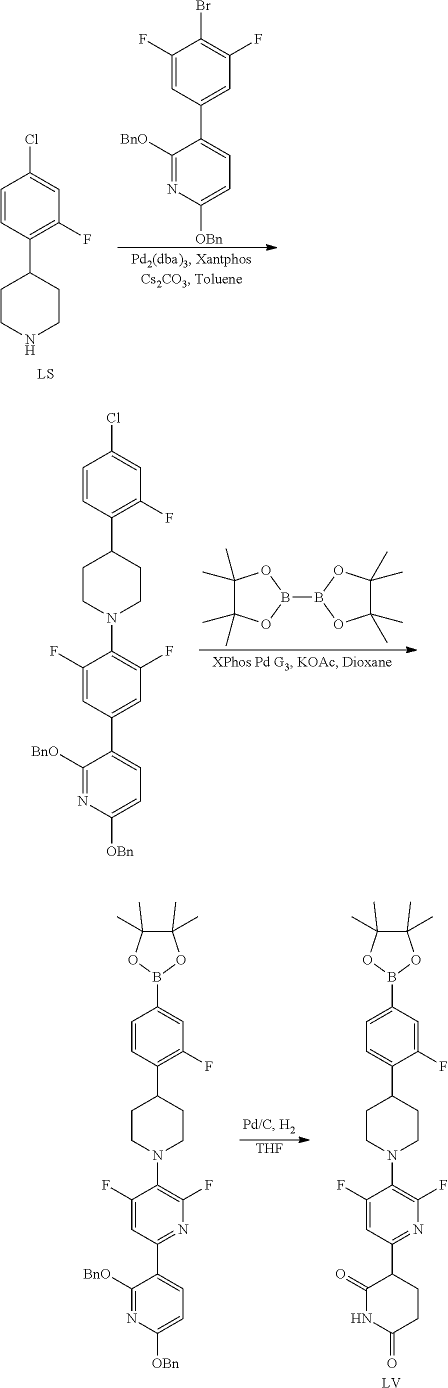

### Step 1—2,6-Bis(benzyloxy)-3-(4-(4-(4-chloro-2-fluorophenyl)piperidin-1-yl)-3,5-difluorophenyl)pyridine

A mixture of 4-(4-chloro-2-fluorophenyl)piperidine (400 mg, 1.9 mmol, Intermediate KS), 2,6-bis(benzyloxy)-3-(4-bromo-3,5-difluorophenyl)pyridine (902 mg, 1.87 mmol, Intermediate LS), Pd₂(dba)₃ (119 mg, 131 μmol), Xantphos (162 mg, 280 μmol) and Cs₂CO₃ (1.52 g, 4.68 mmol) in toluene (10 mL) was degassed and purged with N₂ three times. Then the mixture was stirred at 100° C. for 12 hrs under N₂ atmosphere. On completion, the mixture was quenched with water (20 mL) and extracted with ethyl acetate (2×20 mL). The combined organic layers were washed with brine (40 mL) and dried over anhydrous sodium sulfate, filtered and the filtrate was concentrated in vacuo to give the crude residue. The residue was purified by column chromatography (SiO₂, Petroleum ether:Ethyl acetate=1/0) to give the title compound (460 mg, 40% yield) as a yellow solid. LC-MS (ESI⁺) m/z 615.1 (M+H)⁺.

### Step 2—2,6-Bis(benzyloxy)-3-(3,5-difluoro-4-(4-(2-fluoro-4-(4,4,5,5-tetramethyl-1,3,2-dioxaborolan-2-yl)phenyl)piperidin-1-yl)phenyl)pyridine

A mixture of 2,6-bis(benzyloxy)-3-(4-(4-(4-chloro-2-fluorophenyl)piperidin-1-yl)-3,5-difluorophenyl)pyridine (400 mg, 650 μmol), 4,4,5,5-tetramethyl-2-(4,4,5,5-tetramethyl-1,3,2-dioxaborolan-2-yl)-1,3,2-dioxaborolane (247 mg, 975 μmol, CAS #73183-34-3), XPhos Pd G3 (55.0 mg, 65.0 μmol), and AcOK (191 mg, 1.95 mmol) in dioxane (10 mL) was degassed and purged with N₂ three times. Then the mixture was stirred at 80° C. for 2 hrs under N₂ atmosphere. On completion, the mixture was quenched with water (10 mL) and extracted with ethyl acetate (2×20 mL). The combined organic layers were washed with brine (40 mL) and dried over anhydrous sodium sulfate, filtered and the filtrate was concentrated in vacuo to give the crude residue. The residue was purified by column chromatography (SiO₂, Petroleum ether:Ethyl acetate=1/0) to give the title compound (400 mg, 87% yield) as a yellow solid. LC-MS (ESI⁺) m/z 707.2 (M+H)⁺.

### Step 3—3-(3,5-Difluoro-4-(4-(2-fluoro-4-(4,4,5,5-tetramethyl-1,3,2-dioxaborolan-2-yl)phenyl)piperidin-1-yl)phenyl)piperidine-2,6-dione

To a solution of 2,6-bis(benzyloxy)-3-(3,5-difluoro-4-(4-(2-fluoro-4-(4,4,5,5-tetramethyl-1,3,2-dioxaborolan-2-yl)phenyl)piperidin-1-yl)phenyl)pyridine (300 mg, 424 μmol) in THF (20 mL) was added Pd/C (10 wt %, 400 mg) under N₂ atmosphere. The suspension was degassed and purged with H₂ three times. The mixture was then stirred under H₂ (15 psi) at 25° C. for 12 hrs. On completion, the mixture was filtered and the filtrate was concentrated in vacuo to give the crude residue. The residue was purified by column chromatography (SiO₂, Petroleum ether:Ethyl acetate=1/0 to 2/1) to give the title compound (200 mg, 89% yield) as a white solid. LC-MS (ESI⁺) m/z 529.1 (M+H)⁺.

### 3-(4-Bromo-3-methyl-1H-indazol-1-v)-1-(4-methoxybenzyl)piperidine-2,6-dione (Intermediate LW)

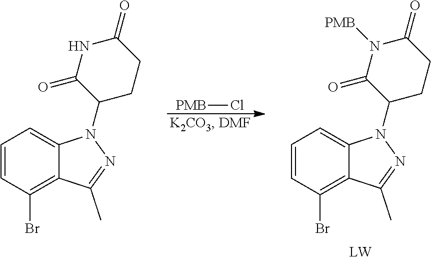

To a solution of 3-(4-bromo-3-methyl-1H-indazol-1-yl)piperidine-2,6-dione (2 g, 6.21 mmol, synthesized via Step 1 of Intermediate AB) in DMF (20 mL) was added PMB-C1 (972 mg, 6.21 mmol, 842 μL) and K₂CO₃ (2.57 g, 18.6 mmol). Then the mixture was stirred at 40° C. for 12 hrs. On completion, the reaction mixture was quenched with water (30 mL), then filtered and filter cake was dried in vacuo to give the title compound (1.5 g) as a purple solid. LC-MS (ESI⁺) m/z 443.9 (M+H)⁺.

### 3-(4-(4-(4-Chloro-2,3-difluorophenyl)piperidin-1-yl)-3-methyl-1H-indazol-1-yl)piperidine-2,6-dione (Intermediate LX)

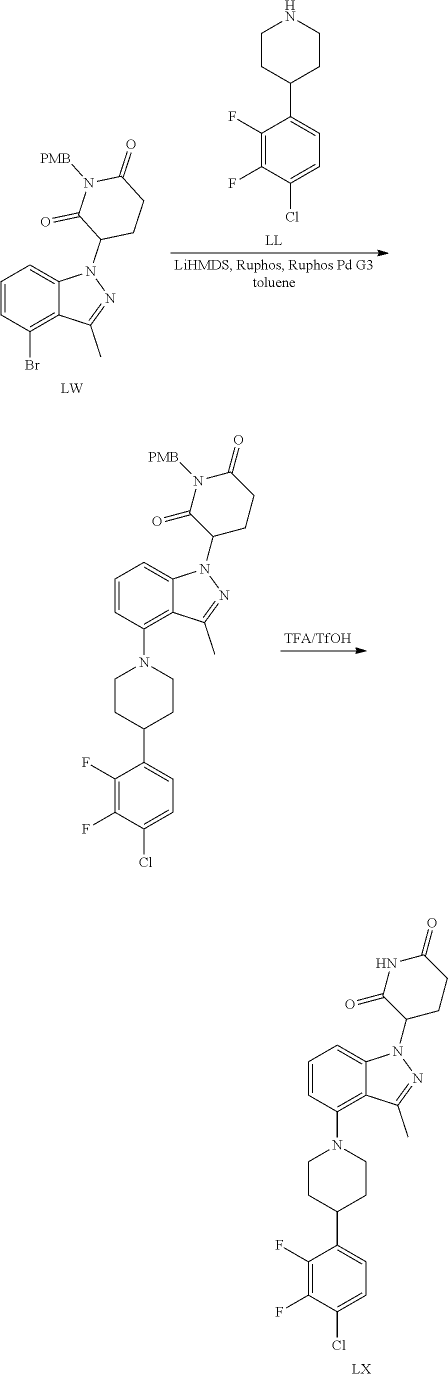

### Step 1—3-(4-(4-(4-Chloro-2,3-difluorophenyl)piperidin-1-yl)-3-methyl-1H-indazol-1-yl)-1-(4-methoxybenzyl)piperidine-2,6-dione

A mixture of 4-(4-chloro-2,3-difluorophenyl)piperidine (50 mg, 220 μmol, Intermediate LL), 3-(4-bromo-3-methyl-1H-indazol-1-yl)-1-(4-methoxybenzyl)piperidine-2,6-dione (95.5 mg, 216 μmol, Intermediate LW), RuPhos (20.1 mg, 43.2 μmol), Pd₂(dba)₃ (19.8 mg, 21.6 μmol) and Cs₂CO₃ (211 mg, 648 μmol) in dioxane (2 mL) was degassed and purged with N₂ three times. Then the mixture was stirred at 100° C. for 1 hr under N₂ atmosphere. On completion, the reaction mixture was quenched with water (30 mL) and extracted with dichloromethane (3×30 mL). The combined organic layers were washed with aqueous NaCl (30 mL) and dried over anhydrous sodium sulfate, filtered and the filtrate was concentrated in vacuo to give the title compound (80 mg) as a brown oil. LC-MS (ESI⁺) m/z 593.2 (M+H)⁺.

### Step 2—3-(4-(4-(4-Chloro-2,3-difluorophenyl)piperidin-1-yl)-3-methyl-1H-indazol-1-yl)piperidine-2,6-dione

To a solution of 3-(4-(4-(4-chloro-2,3-difluorophenyl)piperidin-1-yl)-3-methyl-1H-indazol-1-yl)-1-(4-methoxybenzyl)piperidine-2,6-dione (110 mg, 148 μmol) in TFA (1.5 mL) was added TfOH (848 mg, 5.65 mmol, 0.5 mL). The mixture was then stirred at 50° C. for 12 hrs. On completion, the reaction mixture was concentrated under reduced pressure to remove solvent. The crude residue was purified by reversed-phase HPLC (0.1% FA condition) to give the title compound (39 mg, 56% yield) as a white solid. LC-MS (ESI⁺) m/z 473.0 (M+H)⁺.

### 9-((Tert-butyldimethylsilyl)oxy)-3-azaspiro[5.5]undecane (Intermediate LY)

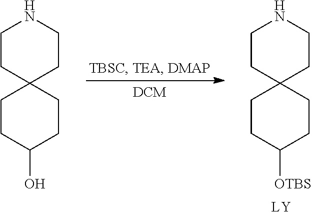 [SMILES: OC1CCC2(CCNCC2)CC1 ; CC(C)(C)[Si](C)(C)OC1CCC2(CCNCC2)CC1]

To a solution of 3-azaspiro[5.5]undecan-9-ol (14 g. 68.0 mmol, HCl. CAS #1820619-77-9) in DCM (10 mL) was added DMAP (1.66 g, 13.6 mmol), TBSCl (15.3 g, 102 mmol) and TEA (68.8 g, 680 mmol) at 0° C. The mixture was then stirred at 40° C. for 24 hrs. On completion, the reaction mixture was quenched with water (200 mL) at 25° C., and then extracted with dichloromethane (3×200 mL). The combined organic layers were dried over anhydrous sodium sulfate, filtered and the filtrate was concentrated in vacuo to give the crude residue. The residue was purified by column chromatography (SiO₂, Petroleum ether:Ethyl acetate=1/0 to 0/1, Methanol:Dichloromethane=1/10 to 1/0) to give the title compound (8 g, 45% yield) as a gray solid. LC-MS (ESI⁺) m/z 284.3 (M+H)⁺.

### 3-(1-(1-(4-Methoxybenzyl)-2,6-dioxopiperidin-3-yl)-3-methyl-2-oxo-2,3-dihydro-1H-benzo[d]imidazol-4-yl)-3-azaspiro[5.5]undec-8-en-9-yl trifluoromethanesulfonate (Intermediate LZ)

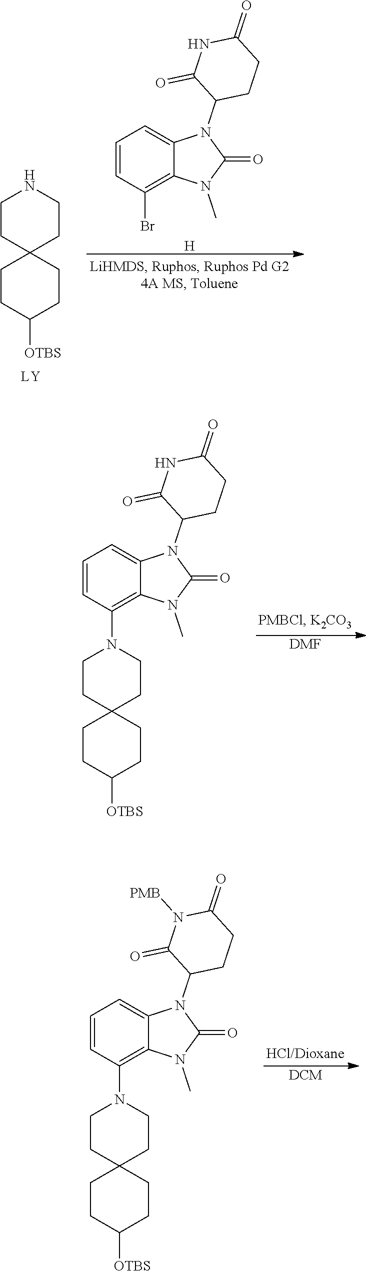

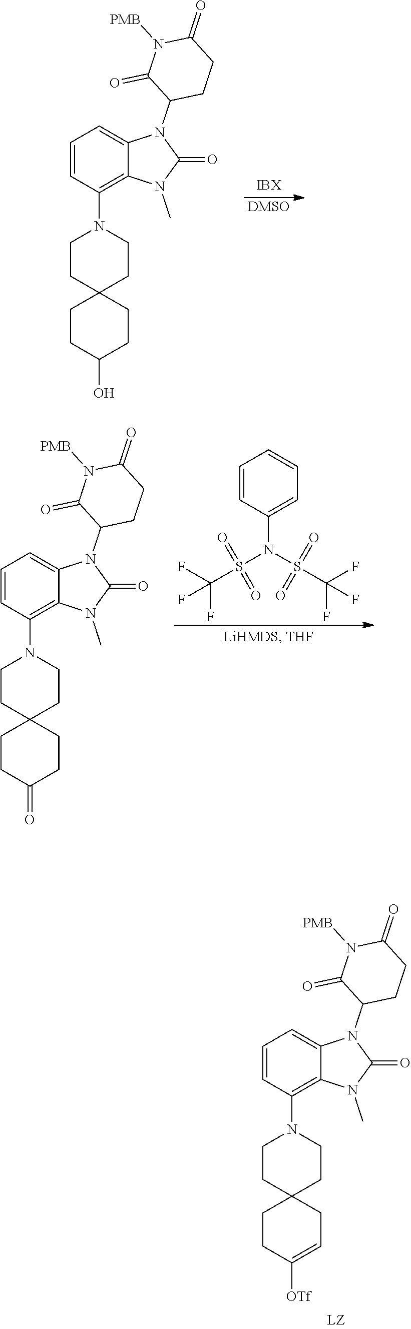

### Step 1—3-(4-(9-((Tert-butyldimethylsilyl)oxy)-3-azaspiro[5.5]undecan-3-yl)-3-methyl-2-oxo-2,3-dihydro-1H-benzo[d]imidazol-1-yl)piperidine-2,6-dione

To a solution of 9-((tert-butyldimethylsilyl)oxy)-3-azaspiro[5.5]undecane (7 g, 24.6 mmol, Intermediate LY), 3-(4-bromo-3-methyl-2-oxo-2,3-dihydro-1H-benzo[d]imidazol-1-yl)piperidine-2,6-dione (10.0 g, 29.6 mmol, Intermediate H), 4 Å molecular sieves (15 g) in toluene (100 mL) was degassed and purged with N₂ three times. Then was added dropwise LiHMDS (1 M, 148 mL) at 0° C. After addition, [2-(2-aminophenyl)phenyl]-chloro-palladium; dicyclohexyl-[2-(2,6-diisopropoxyphenyl)phenyl]phosphane (1.92 g, 2.47 mmol) and RuPhos (1.73 g, 3.70 mmol) was added at 0° C. The resulting mixture was stirred at 100° C. for 2 hrs under N₂ atmosphere. On completion, the reaction mixture was quenched by addition of FA at 0° C. until the pH 4, and then filtered and the filtrate was concentrated in vacuo to give the crude residue. The residue was purified by column chromatography (SiO₂, Petroleum ether:Ethyl acetate=5/1 to 1/1, Dichloromethan:EA=1/1) to give the title compound (9 g, 50% yield) as a brown solid. LC-MS (ESI⁺) m/z 541.3 (M+H)⁺.

### Step 2—3-(4-(9-((Tert-butyldimethylsilyl)oxy)-3-azaspiro[5.5]undecan-3-yl)-3-methyl-2-oxo-2,3-dihydro-1H-benzo[d]imidazol-1-yl)-1-(4-methoxybenzyl)piperidine-2,6-dione

To a solution of 3-(4-(9-((tert-butyldimethylsilyl)oxy)-3-azaspiro[5.5]undecan-3-yl)-3-methyl-2-oxo-2,3-dihydro-1H-benzo[d]imidazol-1-yl)piperidine-2,6-dione (7.5 g, 14 mmol) in DMF (90 mL) was added PMB-C1 (2.61 g, 16.6 mmol, 2.26 mL) and K₂CO₃ (3.83 g, 27.7 mmol). The mixture was then stirred at 60° C. for 2 hrs. On completion, the reaction mixture was added diluted with water (200 mL) and extracted with ethyl acetate (3×250 mL). The combined organic layers were dried over anhydrous sodium sulfate, filtered and the filtrate was concentrated in vacuo to give the crude residue. The residue was purified by column chromatography (SiO₂, Petroleum ether/Ethyl acetate=5/1 to 1/1, DCM:EA=1:1) to give the title compound (9 g, 70% yield) as a white solid. LC-MS (ESI⁺) m/z 661.4 (M+H)⁺.

### Step 3—3-(4-(9-Hydroxy-3-azaspiro[5.5]undecan-3-yl)-3-methyl-2-oxo-2,3-dihydro-1H-benzo[d]imidazol-1-yl)-1-(4-methoxybenzyl)piperidine-2,6-dione

To a solution of 3-(4-(9-((tert-butyldimethylsilyl)oxy)-3-azaspiro[5.5]undecan-3-yl)-3-methyl-2-oxo-2,3-dihydro-1H-benzo[d]imidazol-1-yl)-1-(4-methoxybenzyl)piperidine-2,6-dione (9 g, 14 mmol) in DCM (5 mL) was added HCl/dioxane (2 M, 45.0 mL). The mixture was then stirred at 25° C. for 2 hrs. On completion, the reaction mixture was concentrated under reduced pressure to give a residue. The crude residue was purified by reversed-phase HPLC (0.8 g/L ammonium bicarbonate) to give the title compound (6 g, 50% yield) as a white solid. LC-MS (ESI) m/z 547.2 (M+H)⁺.

### Step 4—1-(4-Methoxybenzyl)-3-(3-methyl-2-oxo-4-(9-oxo-3-azaspiro[5.5]undecan-3-yl)-2,3-dihydro-1H-benzo[d]imidazol-1-3l)piperidine-2,6-dione

To a solution of 3-(4-(9-hydroxy-3-azaspiro[5.5]undecan-3-yl)-3-methyl-2-oxo-2,3-dihydro-1H-benzo[d]imidazol-1-yl)-1-(4-methoxybenzyl)piperidine-2,6-dione (6 g, 10 mmol) in DMSO (60 mL) was added IBX (4.61 g, 16.4 mmol). The mixture was then stirred at 30° C. for 1 hr. On completion, to the reaction mixture was added water (150 mL) and the mixture was extracted with ethyl acetate (3×150 mL). The combined organic layers were dried over anhydrous sodium sulfate, filtered and the filtrate was concentrated in vacuo to give the crude residue. The residue was purified by column chromatography (SiO₂, Petroleum ether/Ethyl acetate=5/1 to 1/1, DCM:EA=1:1) to give the title compound (5 g, 75% yield) as a white solid. LC-MS (ESI⁺) m/z 545.2 (M+H)⁺.

### Step 5—3-(1-(1-(4-Methoxybenzyl)-2,6-dioxopiperidin-3-yl)-3-methyl-2-oxo-2,3-dihydro-1H-benzo[d]imidazol-4-yl)-3-azaspiro[5.5]undec-8-en-9-yl trifluoromethanesulfonate

To a solution of 1-(4-methoxybenzyl)-3-(3-methyl-2-oxo-4-(9-oxo-3-azaspiro[5.5]undecan-3-yl)-2,3-dihydro-1H-benzo[d]imidazol-1-yl)piperidine-2,6-dione (5 g, 9 mmol) in THF (50 mL) was added dropwise LiHMDS (1 M, 18.3 mL) at 0° C. under N₂ atmosphere. After addition, 1,1,1-trifluoro-N-phenyl-N-((trifluoromethyl)sulfonyl)methanesulfonamide (4.26 g, 11.9 mmol, CAS #37595-74-7) in THF (50 mL) was added dropwise at 0° C. The resulting mixture was stirred at 0° C. for 3 hrs under N₂ atmosphere. On completion, the reaction mixture was added NH₄Cl (150 mL), then diluted with water (100 mL) and extracted with ethyl acetate (3×200 mL). The combined organic layers were dried over anhydrous sodium sulfate, filtered and the filtrate was concentrated in vacuo to give the crude residue. The residue was purified by column chromatography (SiO₂, Petroleum ether/Ethyl acetate=5/1 to 1/1) to give the title compound (2.5 g, 36% yield) as a white solid. LC-MS (ESI⁺) m/z 677.2 (M+H)⁺.

### 6-Chloro-7-fluoro-N,N-dimethyl-4-(4,4,5,5-tetramethyl-1,3,2-dioxaborolan-2-yl)-1H-indole-2-carboxamide (Intermediate MA)

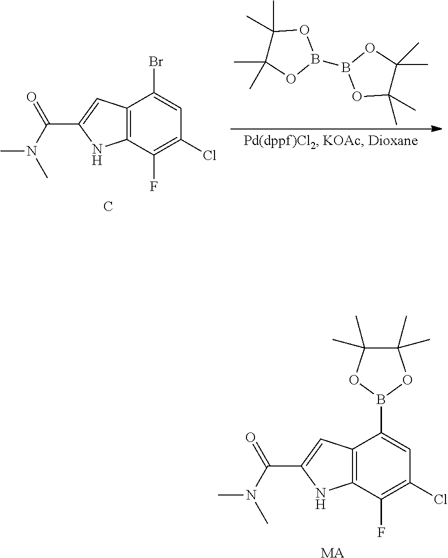

A mixture of 4-bromo-6-chloro-7-fluoro-N,N-dimethyl-1H-indole-2-carboxamide (1.7 g, 5.3 mmol, Intermediate C), 4,4,5,5-tetramethyl-2-(4,4,5,5-tetramethyl-1,3,2-dioxaborolan-2-yl)-1,3,2-dioxaborolane (2.70 g, 10.6 mmol, CAS #73183-34-3), KOAc (1.57 g, 15.9 mmol), and Pd(dppf)Cl₂ (583 mg, 797 μmol) in dioxane (20 mL) was degassed and purged with N₂ three times. Then the mixture was stirred at 80° C. for 2 hrs under N₂ atmosphere. On completion, the reaction mixture was concentrated under reduced pressure to give a residue. The residue was purified by column chromatography (SiO₂, Petroleum ether/Ethyl acetate=10/1 to 3/1) to give the title compound (1.4 g, 50% yield) as a brown solid. LC-MS (ESI⁺) m/z 366.8 (M+H)⁺.

### 3-((3-Fluoro-4-(4,4,5,5-tetramethyl-1,3,2-dioxaborolan-2-yl)phenyl)amino)piperidine-2,6-dione (Intermediate MB)

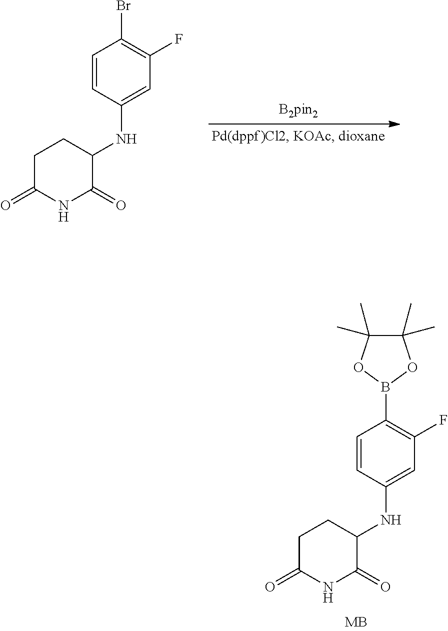 [SMILES: O=C1CCC(Nc2ccc(Br)c(F)c2)C(=O)N1 ; CC1(C)OB(c2ccc(NC3CCC(=O)NC3=O)cc2F)OC1(C)C]

A mixture of 3-((4-bromo-3-fluorophenyl)amino)piperidine-2,6-dione (1 g, 3.32 mmol, synthesized via Step 1 of Intermediate BG), 4,4,5,5-tetramethyl-2-(4,4,5,5-tetramethyl-1,3,2-dioxaborolan-2-yl)-1,3,2-dioxaborolane (843 mg, 3.32 mmol), KOAc (651 mg, 6.64 mmol), and Pd(dppf)Cl₂ (243 mg, 332 μmol) in dioxane (40 mL) was degassed and purged with N₂ three times. Then the mixture was stirred at 80° C. for 2 h under N₂ atmosphere. On completion, the reaction mixture was diluted with H₂O (100 mL) and extracted with EA (100 mL×3). The combined organic layers were washed with brine (50 mL×2), dried over Na₂SO₄, filtered and concentrated under reduced pressure to give a residue. The residue was purified by prep-HPLC (FA condition) to give the title compound (222 mg, 17% yield) as a white solid. LC-MS (ESI⁺) m/z 444.8 (M+H)⁺.

### 1-(4-Chloro-2,5-difluorophenyl)-1,2,3,6-tetrahydropyridin-4-yl trifluoromethanesulfonate (Intermediate MC)

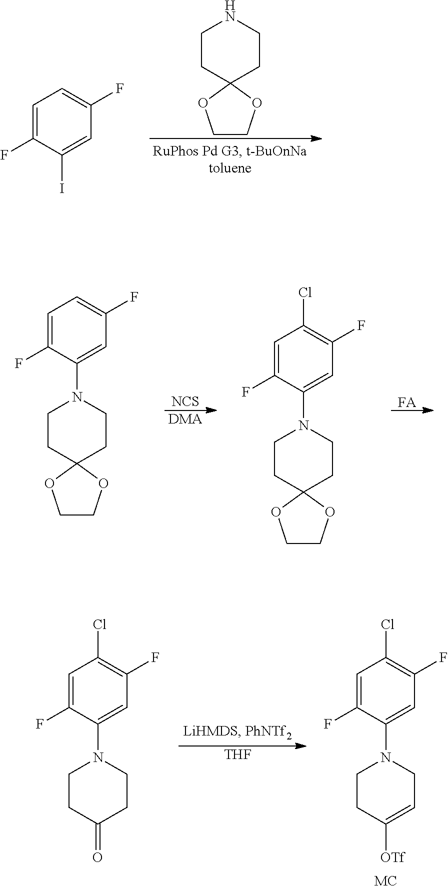

### Step 1—8-(2,5-Difluorophenyl)-1,4-dioxa-8-azaspiro[4.5]decane

A mixture of 1,4-difluoro-2-iodobenzene (2 g, 8 mmol, CAS #2265-92-1), 1,4-dioxa-8-azaspiro[4.5]decane (3.58 g, 25.0 mmol, CAS #177-11-7), RuPhos Pd G3 (139.4 mg, 166.7 μmol), and t-BuONa (961 mg, 10.0 mmol) in toluene (20 mL) was degassed and purged with N₂ three times. Then the mixture was stirred at 100° C. for 2 hrs under N₂ atmosphere. The mixture was diluted with water (50 mL) and extracted with ethyl acetate (50 mL×2), dried over sodium sulfate, filtered and concentrated under reduced pressure. The residue was purified by silica gel column chromatography (petroleum ether/ethyl acetate=1/0˜10/1) to give the title compound (1.47 g, 66% yield) as a yellow solid. LC-MS (ESI⁺) m/z 256.0 (M+H)⁺.

### Step 2—8-(4-Chloro-2,5-difluorophenyl)-1,4-dioxa-8-azaspiro[4.5]decane

A mixture of 8-(2,5-difluorophenyl)-1,4-dioxa-8-azaspiro[4.5]decane (1.37 g. 5.37 mmol) and NCS (717 mg, 5.37 mmol) in DMA (15 mL) was degassed and purged with N₂ three times. Then the mixture was stirred at 40° C. for 12 h under N₂ atmosphere. On completion, the mixture was diluted with water (50 mL) and extracted with ethyl acetate (50 mL×2), dried over sodium sulfate, filtered and concentrated under reduced pressure. The residue was purified by silica gel column chromatography (petroleum ether/ethyl acetate=1/0˜30/1) to give the title compound (822 mg, 48% yield) as a yellow solid. LC-MS (ESI⁺) m/z 290.0 (M+H)⁺.

### Step 3—1-(4-Chloro-2,5-difluorophenyl)piperidin-4-one

A solution of 8-(4-chloro-2,5-difluorophenyl)-1,4-dioxa-8-azaspiro[4.5]decane (4 g, 14 mmol) in HCOOH (40 mL) was stirred at 80° C. for 4 h. On completion, the mixture was filtered and concentrated under reduced pressure to give a residue. The residue was purified by prep-HPLC to give the title compound (1.44 g, 41% yield) as a white solid. LC-MS (ESI⁺) m/z 246.0 (M+H)⁺.

### Step 4—1-(4-Chloro-2,5-difluorophenyl)-1,2,3,6-tetrahydropyridin-4-yl trifluoromethanesulfonate

To a solution of 1-(4-chloro-2,5-difluoro-phenyl)piperidin-4-one (1.4 g, 5.7 mmol) in THF (20 mL) was added LiHMDS (1 M, 11.40 mL) at −78° C., and stirred for 0.5 hr. Then 1,1,1-trifluoro-N-phenyl-N-(trifluoromethylsulfonyl)methanesulfonamide (3.05 g, 8.55 mmol) in THF (20 mL) was added at −78° C., and the mixture was stirred for 0.5 hr. On completion, the mixture was filtered and concentrated under reduced pressure to give a residue. The residue was purified by silica gel column chromatography (petroleum ether/ethyl acetate=50/1˜30/1) to give the title compound (435 mg, 16% yield) as a white solid. LC-MS (ESI⁺) m/z 377.9 (M+H)⁺.

### 3-((4-(1-(4-chloro-2,5-difluorophenyl)-1,2,3,6-tetrahydropyridin-4-yl)-3-fluorophenyl)amino)piperidine-2,6-dione (Intermediate MD)

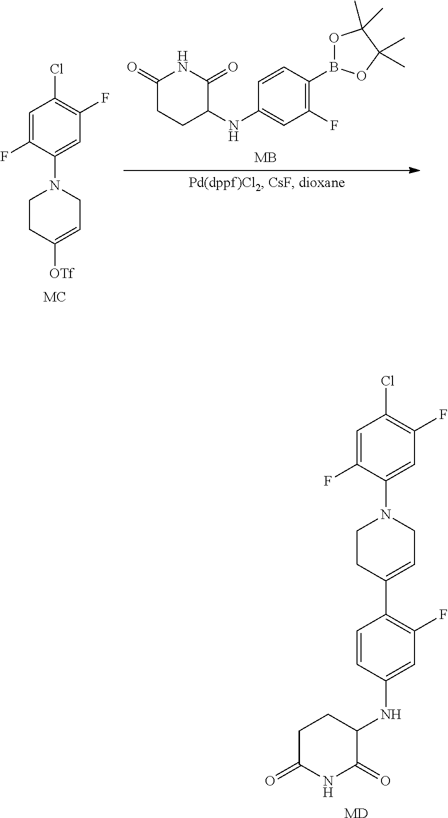

A mixture of 1-(4-chloro-2,5-difluorophenyl)-1,2,3,6-tetrahydropyridin-4-yl trifluoromethanesulfonate (300 mg, 794 μmol, Intermediate MC), 3-((3-fluoro-4-(4,4,5,5-tetramethyl-1,3,2-dioxaborolan-2-yl)phenyl)amino)piperidine-2,6-dione (276 mg, 794 μmol, Intermediate MB), Pd(dppf)Cl₂ (58.1 mg, 79.3 μmol), and CsF (362 mg, 2.38 mmol) in dioxane (3 mL) and H₂O (0.5 mL) was degassed and purged with N₂ three times. Then the mixture was stirred at 60° C. for 2 hrs under N₂ atmosphere. On completion, the mixture was diluted with water (50 mL) and extracted with ethyl acetate (50 mL×2), dried over sodium sulfate, filtered and concentrated under reduced pressure. The residue was purified by silica gel column chromatography (petroleum ether/ethyl acetate=4/1˜2/1) to give the title compound (172 mg, 41% yield) as a yellow solid. LC-MS (ESI⁺) m/z 449.8 (M+H)⁺.

### 3-((4-(1-(2,5-Difluoro-4-(4,4,5,5-tetramethyl-1,3,2-dioxaborolan-2-yl)phenyl)piperidin-4-yl)-3-fluorophenyl)amino)piperidine-2,6-dione (Intermediate ME)

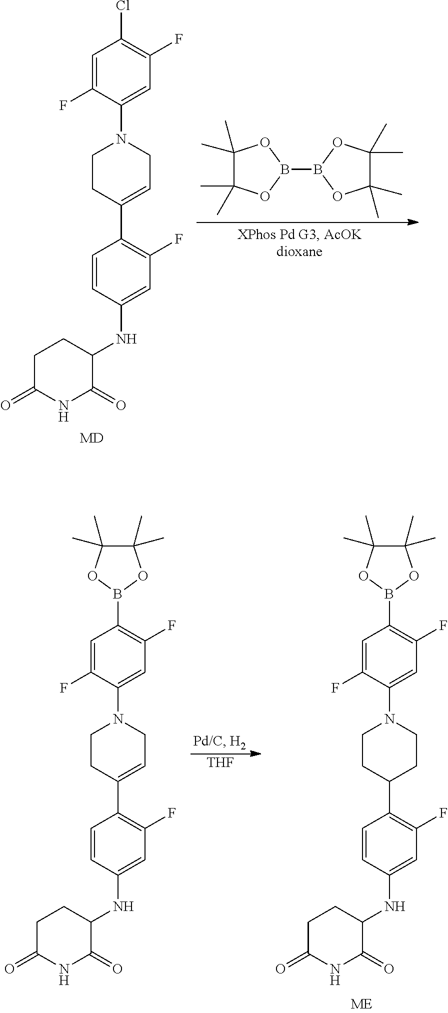

### Step 1—3-((4-(1-(2,5-Difluoro-4-(4,4,5,5-tetramethyl-1,3,2-dioxaborolan-2-yl)phenyl)-1,2,3,6-tetrahydropyridin-4-yl)-3-fluorophenyl)amino)piperidine-2,6-dione

A mixture of 3-((4-(1-(4-chloro-2,5-difluorophenyl)-1,2,3,6-tetrahydropyridin-4-yl)-3-fluorophenyl)amino)piperidine-2,6-dione (172 mg, 382.4 μmol, Intermediate MD), 4,4,4′,4′,5,5,5′,5′-octamethyl-2,2′-bi(1,3,2-dioxaborolane) (97.1 mg, 382.4 μmol), XPhos Pd G3 (32.36 mg, 38.23 μmol), and AcOK (112.57 mg, 1.15 mmol) in dioxane (3 mL) was degassed and purged with N₂ three times. Then the mixture was stirred at 80° C. for 1 hr under N₂ atmosphere. On completion, the mixture was diluted with water (50 mL) and extracted with ethyl acetate (50 mL×2), dried over sodium sulfate, filtered and concentrated under reduced pressure. The residue was purified by silica gel column chromatography (petroleum ether/ethyl acetate=4/1˜2/1). to give the title compound (72 mg, 31% yield) as a yellow solid. LC-MS (ESI⁺) m/z 459.1 (M+H)⁺.

### Step 2—3-((4-(1-(2,5-Difluoro-4-(4,4,5,5-tetramethyl-1,3,2-dioxaborolan-2-yl)phenyl)piperidin-4-yl)-3-fluorophenyl)amino)piperidine-2,6-dione

A mixture of 3-((4-(1-(2,5-difluoro-4-(4,4,5,5-tetramethyl-1,3,2-dioxaborolan-2-yl)phenyl)-1,2,3,6-tetrahydropyridin-4-yl)-3-fluorophenyl)amino)piperidine-2,6-dione (72 mg, 133 μmol), and Pd/C (72 mg, 67.7 μmol, 10 wt %) in THF (4 mL) was degassed and purged with H₂ three times. Then the mixture was stirred at 30° C. for 12 hrs under H₂ (40 psi) atmosphere. The mixture was filtered and the filtrate was concentrated under reduced pressure to give the title compound (55 mg, 76% yield) as a white solid. LC-MS (ESI⁺) m/z 461.9 (M+H)⁺.

### 3-(4-(4-(4-Chlorophenyl)piperidin-1-yl)-2,5-difluorophenyl)piperidine-2,6-dione (Intermediate MF)

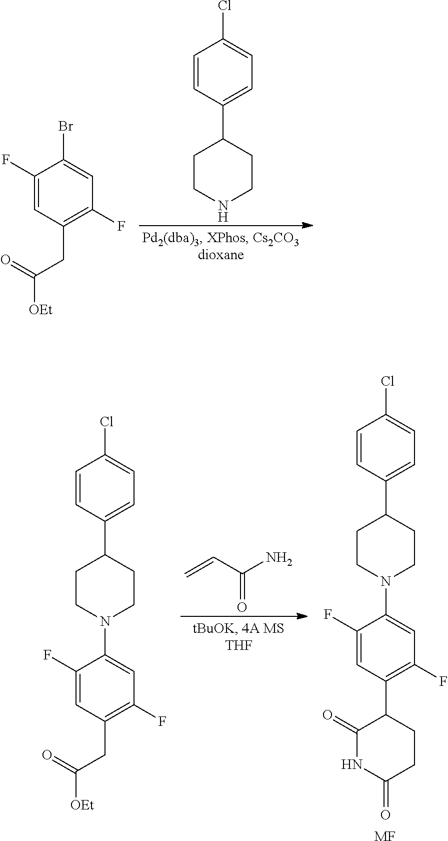

### Step 1—Ethyl 2-(4-(4-(4-chlorophenyl)piperidin-1-yl)-2,5-difluorophenyl)acetate

To a solution of ethyl 2-(4-bromo-2,5-difluorophenyl)acetate (4 g, 14 mmol, CAS #1807120-48-4) in toluene (80 mL) was added 4-(4-chlorophenyl)piperidine (2.55 g, 13.0 mmol, CAS #26905-02-2), Cs₂CO₃ (12.7 g, 39.0 mmol), XPhos (621 mg, 1.30 mmol) and Pd₂(dba)₃ (596 mg, 651 μmol). The reaction was then stirred at 100° C. for 2 hrs. On completion, the reaction mixture was quenched with FA until the pH=7 and extracted by ethyl acetate (3×80 mL). The combined organic layers were washed with brine (100 mL) and dried over anhydrous sodium sulfate, filtered and the filtrate was concentrated in vacuo to give the crude residue. The residue was purified by column chromatography (SiO₂, Petroleum ether:Ethyl acetate=10/1 to 2/1) to give the title compound (4 g, 60% yield) as a brown solid. LC-MS (ESI⁺) m/z 394.0 (M+H)⁺.

### Step 2—3-(4-(4-(4-Chlorophenyl)piperidin-1-yl)-2,5-difluorophenyl)piperidine-2,6-dione

To a solution of ethyl 2-(4-(4-(4-chlorophenyl)piperidin-1-yl)-2,5-difluorophenyl)acetate (2 g, 5 mmol) in THF (30 mL) was added acrylamide (360 mg, 5.08 mmol, 350 μL), 4 Å molecular sieves (4 g) and tBuOK (626 mg, 5.59 mmol). The reaction was then stirred at 25° C. for 4 hrs. On completion, the reaction mixture was filtered and the filtrate was concentrated in vacuo to give the crude residue. The residue was purified by column chromatography (SiO₂, Petroleum ether:Ethyl acetate=10/1 to 2/1) to give the title compound (1 g, 38% yield) as a yellow solid. LC-MS (ESI⁺) m/z 419.0 (M+H).

### Ethyl 2-[5-(tert-butoxycarbonylamino)-2-oxo-1-pyridyl]acetate (Intermediate HJ′)

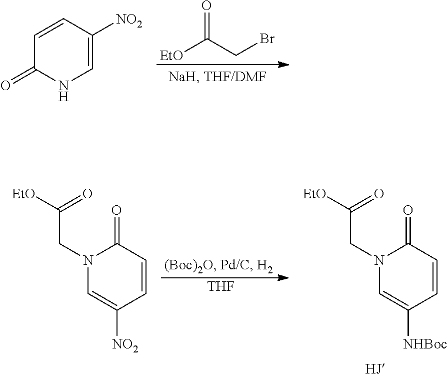 [SMILES: O=c1ccc([N+](=O)[O-])c[nH]1 ; CCOC(=O)CBr ; CCOC(=O)Cn1cc([N+](=O)[O-])ccc1=O ; CCOC(=O)Cn1cc(NC(=O)OC(C)(C)C)ccc1=O ; "THF"]

### Step 1—Ethyl 2-(5-nitro-2-oxo-1-pyridyl)acetate

To a solution of 5-nitro-1H-pyridin-2-one (5.00 g, 35.7 mmol, CAS #5418-51-9) in THF (70 mL) and DMF (10 mL) was added NaH (1.71 g, 42.8 mmol, 60% dispersion in mineral oil), then the mixture was stirred at 0° C. for 1 hr. Next, ethyl 2-bromoacetate (6.56 g, 39 mmol, CAS #105-36-2) was added, and the mixture was stirred at 25° C. for 1 hr. On completion, saturated NH₄Cl was added to the mixture to adjust the pH to 6. Then the mixture was diluted with water (20 mL), filtered to give the filter cake and dried in vacuo. The crude product was purified by column chromatography (SiO₂, PE:EA=1:0 to 0:1) to give the title compound (6.20 g, 77% yield) as white solid. ¹H NMR (400 MHz, DMSO-d₆) δ 9.25 (d, J=3.2 Hz, 1H), 8.20-8.10 (m, 1H), 6.55 (d, J=10.0 Hz, 1H), 4.86 (s, 2H), 4.17 (q, J=7.2 Hz, 2H), 1.21 (t, J=7.2 Hz, 3H); LC-MS (ESI⁺) m/z 226.9 (M+H)⁺.

### Step 2—Ethyl 2-[5-(tert-butoxycarbonylamino)-2-oxo-1-pyridyl]acetate

To a solution of ethyl 2-(5-nitro-2-oxo-1-pyridyl) acetate (1 g, 4.42 mmol) in THF (10 mL) was added (Boc)₂O (2.89 g, 13.2 mmol) and Pd/C (1 g, 4.42 mmol, 10 wt %) under N₂. The suspension was degassed under vacuum and purged with H₂ three times. The mixture was stirred under H₂ (15 psi) at 25° C. for 2 hours. On completion, the reaction mixture was filtered and concentrated in vacuo to give the residue. The residue was purified by column chromatography (SiO₂, PE/EA=20/1 to 1/1) to give the title compound (985 mg, 75% yield) as red solid. ¹H NMR (400 MHz. DMSO-d₆) δ 9.05 (s, 1H), 7.87 (s, 1H), 7.39 (m, 1H), 6.39 (d, J=9.6 Hz, 1H), 4.67 (s, 2H), 4.13 (q, J=7.2 Hz, 2H), 1.45 (s, 9H), 1.20 (t, J=7.2 Hz, 3H); LC-MS (ESI+) m/z 297.0 (M+H)⁺.

### ((2-(2-(2-Bromoethoxy)ethoxy)ethoxy)methyl)benzene (Intermediate HK)

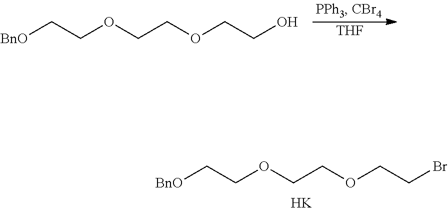

A mixture of PPh₃ (10.9 g, 41.6 mmol), CBr₄ (13.8 g, 41.6 mmol) at 0° C. in THF (50 mL) was degassed and purged with N₂ 3 times, then 2-[2-(2-benzyloxyethoxy)ethoxy]ethanol (5 g, 20 mmol, CAS #55489-58-2) was added and then the mixture was stirred at 25° C. for 2 hrs under N₂ atmosphere. On completion, the reaction mixture was concentrated under reduced pressure to give a residue. The residue was purified by column chromatography (SiO₂, Petroleum ether:Ethyl acetate=10/1 to 3/1) to give the title compound (4 g, 63% yield) as a white oil. ¹H NMR (400 MHz, DMSO-d6) δ=7.44-7.18 (m, 5H), 4.49 (s, 2H), 3.77-3.68 (m, 2H), 3.61-3.52 (m, 10H).

### Ethyl 2-(5-((2-(2-(2-(benzyloxy)ethoxy)ethoxy)ethyl)amino)-2-oxopyridin-1 (2H)-yl)acetate (Intermediate HL)

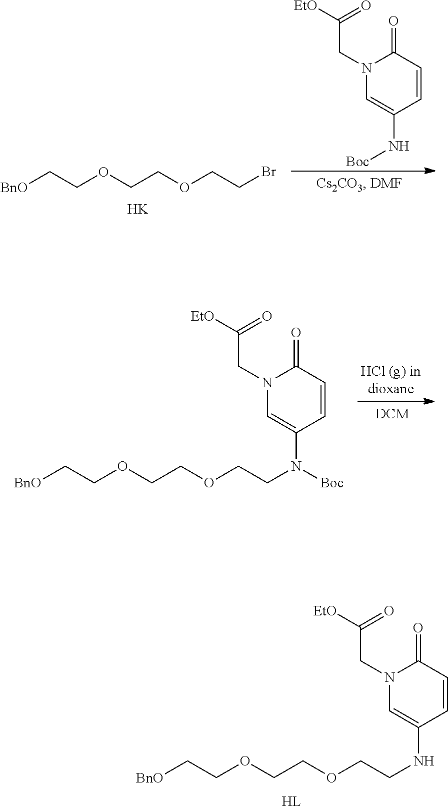

### Step 1—Ethyl 2-(5-((2-(2-(2-(benzyloxy)ethoxy)ethoxy)ethyl)(tert-butoxycarbonyl)amino)-2-oxopyridin-1 (2H)-yl)acetate

To a solution of 2-[2-(2-bromoethoxy)ethoxy]ethoxymethylbenzene (4 g, 10 mmol, Intermediate HK) and ethyl 2-[5-(tert-butoxycarbonylamino)-2-oxo-1-pyridyl]acetate (1.95 g. 6.60 mmol, synthesized via Steps 1-2 of Intermediate HJ′) in ACN (50 mL) was added Cs₂CO₃ (6.45 g, 19.8 mmol). The mixture was then stirred at 50° C. for 16 hrs. On completion, the reaction mixture was diluted with water (100 mL) and extracted with ethyl acetate/dichloromethane (3×100 mL). The combined organic layers were washed with brine (200 mL) and dried over anhydrous sodium sulfate, filtered and the filtrate was concentrated in vacuo to give the crude residue. The residue was purified by column chromatography (SiO₂. Petroleum ether:Ethyl acetate=10/1 to 0/1) to give the title compound as a white oil. The residue was re-purified by reversed-phase HPLC (0.8 g/L ammonium bicarbonate) to give the title compound (2 g, 50% yield) as a blue oil. LC-MS (ESI+) m/z 519.4 (M−55)⁺.

### Step 2—Ethyl 2-(5-((2-(2-(2-(benzyloxy)ethoxy)ethoxy)ethyl)amino)-2-oxopyridin-1 (2H)-yl)acetate

To a solution of ethyl 2-[5-[2-[2-(2-benzyloxyethoxy)ethoxy]ethyl-tert-butoxycarbonyl-amino]-2-oxo-1-pyridyl]acetate (2 g, 3.86 mmol) in DCM (10 mL) was added HCl/dioxane (2 M. 10 mL). The mixture was then stirred at 25° C. for 1 hr. On completion, the reaction mixture was concentrated under reduced pressure to give a residue. The crude residue was purified by reversed-phase HPLC (0.8 g/L ammonium bicarbonate) to give the title compound (1.4 g, 85% yield) as a blue oil. LC-MS (ESI+) m/z 419.2 (M+H)⁺.

### (S)-1-(isoquinolin-4-yl)piperidine-3-carbonyl chloride (Intermediate HM)

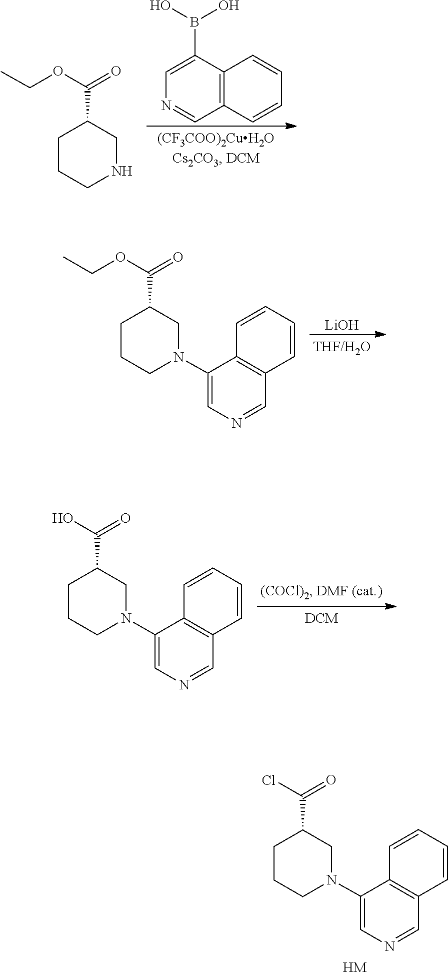

### Step 1—Ethyl (S)-1-(isoquinolin-4-yl)piperidine-3-carboxylate

To a stirred solution of ethyl (S)-piperidine-3-carboxylate (30.00 g, 190.8 mmol), isoquinolin-4-ylboronic acid (49.51 g, 286.24 mmol) and Cs₂CO₃ (186.52 g, 572.47 mmol) in DCM (600 mL) was added Cu(OTf)₂ (138.03 g, 381.7 mmol) at rt under air atmosphere. The resulting mixture was stirred for 16 h at rt under air atmosphere. On completion, the mixture was filtered, and the filter cake was washed with DCM (3×200 mL). The filtrate was concentrated under reduced pressure. The residue was purified by silica gel column chromatography, eluted with petroleum ether/EtOAc (38%), and concentrated under reduced pressure to afford the title compound (12 g, 22% yield) as a purple solid. ¹H NMR (400 MHz, DMSO-d₆) δ 9.02 (s, 1H), 8.20 (s, 1H), 8.13-8.05 (m, 2H), 7.86-7.74 (m, 1H), 7.74-7.61 (m, 1H), 4.12 (q, J=7.2 Hz, 2H), 3.42-3.39 (m, 1H), 3.25-3.16 (m, 1H), 3.13-3.02 (m, 1H), 2.97-2.82 (m, 2H), 2.05-1.95 (m, 1H), 1.95-1.87 (m, 1H), 1.87-1.62 (m, 2H), 1.19 (t, J=7.2 Hz, 3H); LC/MS (ESI, m/z): [(M+1)]⁺=385.2.

### Step 2—(S)-1-(isoquinolin-4-yl)piperidine-3-carboxylic acid

To a stirred solution of ethyl (S)-1-(isoquinolin-4-yl)piperidine-3-carboxylate (12 g, 42 mmol) in THF (100 mL) and H₂O (100 mL) was added LiOH (5.05 g, 211 mmol) in portions at it under nitrogen atmosphere. The resulting mixture was then stirred for 16 hr at rt under nitrogen atmosphere. On completion, the mixture was concentrated under vacuum. The residue was purified by reverse phase Flash chromatography (Column: WelFlash™ C18-I, 20-40 μm, 330 g; Eluent A: Water (10 mmol/L NH₄HCO₃); Eluent B: acetonitrile; Gradient: 5%-30% B in 40 min; Flow rate: 80 mL/min: Detector: 220/254 nm; desired fractions were collected at 20% B) and concentrated under reduced pressure to afford the title compound (9 g, 83% yield) as a yellow solid. LC/MS (ESI, m/z): [(M+1)]⁺=257.0.

### Step 3—(S)-1-(isoquinolin-4-yl)piperidine-3-carbonyl chloride

To a stirred solution of (S)-1-(isoquinolin-4-yl)piperidine-3-carboxylic acid (5 g, 20 mmol) and oxalic dichloride (4.95 g, 39.0 mmol) in DCM (50 mL) was added DMF (0.148 mL, 1.95 mmol) dropwise at rt under nitrogen atmosphere. The resulting mixture was stirred for 1 hr at rt under nitrogen atmosphere. On completion, the mixture was concentrated under vacuum to give the title compound (4 g, 75% yield) as a yellow solid. LC/MS (ESI, m/z): [(M+MeOH)]⁺=271.2.

### Methyl (S)-2-(5-(1-(isoquinolin-4-yl)-N-(2-(2-(2-((methylsulfonyl)oxy)ethoxy)ethoxy)ethyl)piperidine-3-carboxamido)-2-oxopyridin-1 (2H)-yl)acetate (Intermediate HN)

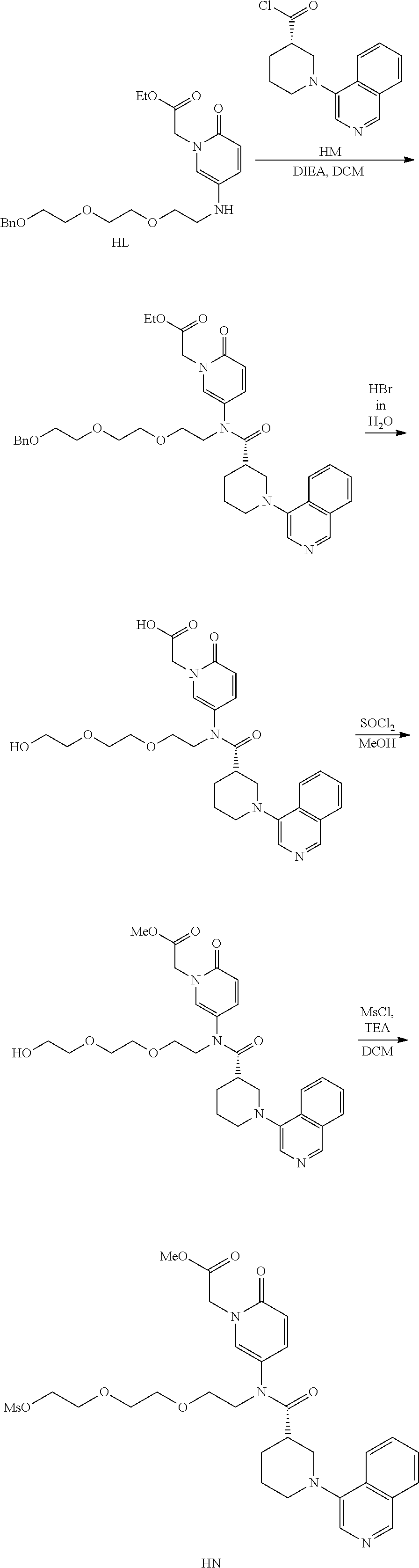

### Step 1—Ethyl (S)-2-(5-(N-(2-(2-(2-(benzyloxy)ethoxy)ethoxy)ethyl)-1-(isoquinolin-4-yl)piperidine-3-carboxamido)-2-oxopyridin-1 (2H)-yl)acetate

To a solution of ethyl 2-[5-[2-[2-(2-benzyloxyethoxy)ethoxy]ethylamino]-2-oxo-1-pyridyl]acetate (380 mg, 908 μmol, Intermediate HL) in dry DCM (2 mL) was added DIEA (586 mg, 4.54 mmol) at 0° C. Then, 1-(4-isoquinolyl)piperidine-3-carbonyl chloride (299 mg, 1.09 mmol, Intermediate HM) in dry DCM (2 mL) was added at 0° C., and the mixture was stirred at 0° C. for 1 hr. On completion, the reaction mixture was concentrated under reduced pressure to remove solvent and give the title compound (250 mg, 42% yield) as a yellow oil. LC-MS (ESI⁺) m/z 657.5 (M+H)⁺.

### Step 2—(S)-2-(5-(N-(2-(2-(2-hydroxyethoxy)ethoxy)ethyl)-1-(isoquinolin-4-yl)piperidine-3-carboxamido)-2-oxopyridin-1 (2H)-yl)acetic acid

A solution of ethyl 2-[5-[2-[2-(2-benzyloxyethoxy)ethoxy]ethyl-[1-(4-isoquinolyl)piperidine-3-carbonyl]amino]-2-oxo-1-pyridyl]acetate (350 mg, 533 μmol) in HBr (5 mL) was stirred at 60° C. for 2 hrs. On completion, the reaction mixture was concentrated under reduced pressure to remove solvent. The residue was purified by prep-HPLC (FA condition) to give the title compound (220 mg, 77% yield) as a white oil. LC-MS (ESI⁺) m/z 539.1 (M+H)⁻.

### Step 3—Methyl (S)-2-(5-(N-(2-(2-(2-hydroxyethoxy)ethoxy)ethyl)-1-(isoquinolin-4-yl)piperidine-3-carboxamido)-2-oxopyridin-1 (2H)-yl)acetate

To a solution of 2-[5-[2-[2-(2-hydroxyethoxy)ethoxy]ethyl-[1-(4-isoquinolyl)piperidine-3-carbonyl]amino]-2-oxo-1-pyridyl]acetic acid (120.00 mg, 223 μmol) in MeOH (1 mL) was added SOCl₂ (1.38 mmol, 0.1 mL) at 0° C. The mixture was then stirred at 70° C. for 1 hr. On completion, the reaction mixture was concentrated under reduced pressure to remove solvent. The residue was purified by prep-HPLC (FA condition) to give the title compound (100 mg. 81% yield) as a blue oil. LC-MS (ESI⁺) i/z 553.3 (M+H)⁺.

### Step 4—methyl (S)-2-(5-(1-(isoquinolin-4-yl)-N-(2-(2-(2-((methylsulfonyl)oxy)ethoxy)ethoxy)ethyl)piperidine-3-carboxamido)-2-oxopyridin-1 (2H)-yl)acetate

To a solution of methyl 2-[5-[2-[2-(2-hydroxyethoxy)ethoxy]ethyl-[1-(4-isoquinolyl)piperidine-3-carbonyl]amino]-2-oxo-1-pyridyl]acetate (20 mg, 36 μmol) in DCM (2 mL) was added TEA (18.3 mg, 180 μmol) and MsCl (8.29 mg, 72.4 μmol) at 0° C. The mixture was stirred at 25° C. for 1 hr. On completion, the reaction mixture was neutralized with 10% HCl and water, and extracted with DCM (20 mL). The residue was purified by column chromatography by prep-TLC (SiO₂, DCM:MeOH=10:1) to give the title compound (10 mg, 44% yield) as a yellow solid. LC-MS (ESI⁺) m/z 631.4 (M+H)⁺.

### 13-Bromo-1-phenyl-2,5,8,11-tetraoxatridecane (Intermediate HO)

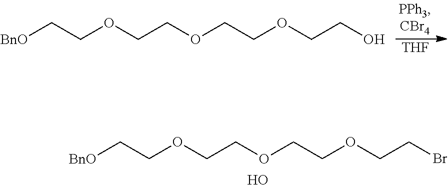

To a solution of 1-phenyl-2,5,8,11-tetraoxatridecan-13-ol (4.00 g, 14.1 mmol, CAS #86259-87-2) in THF (15 mL) was added CBr₄ (9.33 g, 28.1 mmol) and PPh₃ (7.38 g, 28.1 mmol) at 0° C. The mixture was stirred at 25° C. for 4 h. On completion, the reaction mixture was diluted with water (50 mL) and extracted with ethyl acetate (3×50 mL). The combined organic layers were dried over anhydrous Na₂SO₄, filtered and the filtrate was concentrated in vacuo to give the crude residue. The residue was purified by column chromatography (SiO₂, Petroleum ether:Ethyl acetate=100/1 to 0/1) to give the title compound (3.8 g, 78% yield) as a yellow oil. ¹H NMR (400 MHz, DMSO-d₆) δ=7.40-7.24 (m, 5H), 4.49 (s, 2H), 3.76-3.68 (m, 2H), 3.59-3.52 (m, 14H).

### Ethyl 2-(2-oxo-5-((1-phenyl-2,5,8,11-tetraoxatridecan-13-yl)amino)pyridin-1 (2H)-yl)acetate (Intermediate HP)

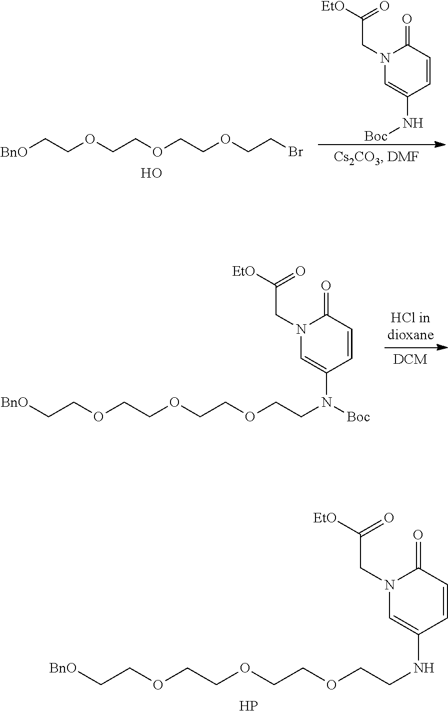

### Step 1—Ethyl 2-(5-((tert-butoxycarbonyl)(1-phenyl-2,5,8,11-tetraoxatridecan-13-yl)amino)-2-oxopyridin-1 (2H)-yl)acetate

To a solution of ethyl 2-(5-((tert-butoxycarbonyl)amino)-2-oxopyridin-1 (2H)-yl)acetate (2 g, 6.75 mmol, synthesized via Steps 1-2 of Intermediate HJ′), 13-bromo-1-phenyl-2,5,8,11-tetraoxatridecane (2.34 g, 6.75 mmol, Intermediate HO) in DMF (5 mL) was added Cs₂CO₃ (6.60 g, 20.2 mmol) and KI (1.12 g, 6.75 mmol). The mixture was then stirred at 70° C. for 12 h. On completion, the reaction mixture was diluted with water (30 mL) and extracted with ethyl acetate (3×30 mL). The combined organic layers were washed with brine (150 mL) and dried over anhydrous Na₂SO₄, filtered and the filtrate was concentrated in vacuo to give the crude residue. The residue was purified by column chromatography (SiO₂, Petroleum ether:Ethyl acetate=100/1 to 0/1) to give the title compound (1 g, 26% yield) as a yellow solid. LC-MS (ESI⁺) m/z 563.1 (M+H)⁺.

### Step 2—Ethyl 2-(2-oxo-5-((I-phenyl-2,5,8,11-tetraoxatridecan-13-yl)amino)pyridin-1 (2H)-yl)acetate

To a solution of ethyl 2-(5-((tert-butoxycarbonyl)(1-phenyl-2,5,8,11-tetraoxatridecan-13-yl)amino)-2-oxopyridin-1 (2H)-yl)acetate (1 g, 2 mmol) in DCM (15 mL) was added HCl/dioxane (4 mL, 4 M). The mixture was then stirred at 25° C. for 2 h. On completion, the reaction mixture was concentrated in vacuo to give the title compound (1 g) as a yellow solid. LC-MS (ESI⁺) m/z 463.2 (M+H)⁺.

### Methyl 2-[5-[[(3S)-1-(4-isoquinolyl)piperidine-3-carbonyl]-[2-[2-[2-(2-methylsulfonyloxyethoxy)ethoxy]ethoxy]ethyl]amino]-2-oxo-1-pyridyl]acetate (Intermediate HQ)

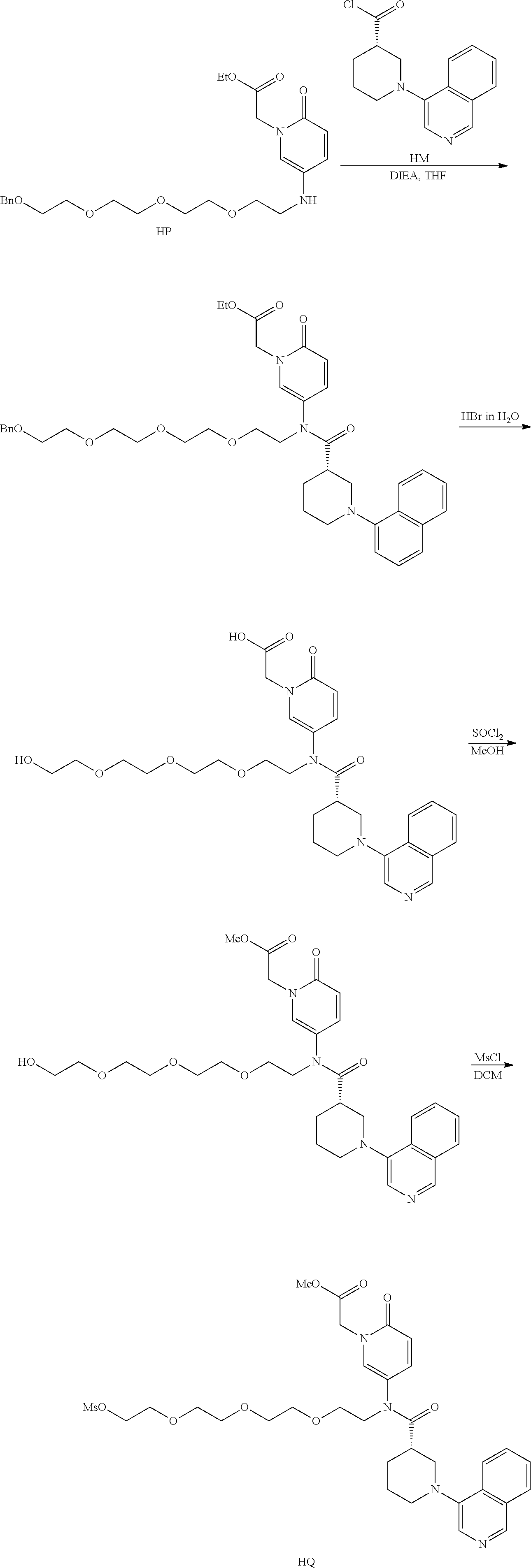

### Step 1—(S)-ethyl 2-(5-(1-(isoquinolin-4-yl)-N-(1-phenyl-2,5,8,11-tetraoxatridecan-13-yl)piperidine-3-carboxamido)-2-oxopyridin-1 (2H)-yl)acetate

To a solution of ethyl 2-(2-oxo-5-((1-phenyl-2,5,8,11-tetraoxatridecan-13-yl)amino)pyridin-1 (2H)-yl)acetate (1 g, 2 mmol, Intermediate HP) in anhydrous DCM (20 mL) was added DIEA (1.40 g, 10.8 mmol, 1.88 mL) then added (S)-1-(isoquinolin-4-yl)piperidine-3-carbonyl chloride (1.19 g, 4.32 mmol, Intermediate HM) in anhydrous DCM (20 mL) at 0° C. under nitrogen atmosphere, then the reaction was stirred at 25° C. for 2 h under nitrogen atmosphere. On completion, the reaction mixture was quenched with water (30 mL) and extracted with dichloromethane (3×50 mL). The combined organic layers were washed with brine (50 mL) and dried over anhydrous sodium sulfate, filtered and the filtrate was concentrated in vacuo to give the crude residue. The residue was purified by column chromatography (SiO₂ Dichloromethane:Methanol=30/1 to 15/1) to give a title compound (600 mg, 34% yield) as a deep blue gum, LC-MS (ESI⁺) m/z 701.3 (M+H)⁺.

### Step 2—(S)-2-(5-(N-(2-(2-(2-(2-hydroxyethoxy)ethoxy)ethoxy)ethyl)-1-(isoquinolin-4-yl)piperidine-3-carboxamido)-2-oxopyridin-1 (2H)-yl)acetic acid

To a solution of (S)-ethyl 2-(5-(1-(isoquinolin-4-yl)-N-(1-phenyl-2,5,8,11-tetraoxatridecan-13-yl)piperidine-3-carboxamido)-2-oxopyridin-1 (2H)-yl)acetate (600 mg, 856 μmol) in HBr (10 mL, 40% solution in water) was stirred at 60° C. for 4 h under nitrogen atmosphere. On completion, the mixture was concentrated under reduced pressure to give the title compound (500 mg) as a brown gum, LC-MS (ESI⁺) m/z 583.2 (M+H)⁺.

### Step 3—(S)-methyl 2-(5-(N-(2-(2-(2-(2-hydroxyethoxy)ethoxy)ethoxy)ethyl)-1-(isoquinolin-4-yl)piperidine-3-carboxamido)-2-oxopyridin-1 (2H)-yl)acetate

To a solution of (S)-2-(5-(N-(2-(2-(2-(2-hydroxyethoxy)ethoxy)ethoxy)ethyl)-1-(isoquinolin-4-yl)piperidine-3-carboxamido)-2-oxopyridin-1 (2H)-yl)acetic acid (1.55 g, 2.59 mmol) in DCM (5 mL) was added HCl/dioxane (8 M, 5 mL). The mixture was then stirred at 25° C. for 2 hrs. On completion, the mixture was adjusted to pH=8 by adding NaHCO₃ solution (15 mL). Then the mixture was extracted with DCM (10 mL×2) and the organic phase was concentrated to give the title compound (400 mg) as a brown gum, LC-MS (ESI⁻) m/z 597.2 (M+H)⁺.

### Step 4—Methyl 2-[5-[[(3S)-1-(4-isoquinolyl)piperidine-3-carbonyl]-[2-[2-[2-(2-methylsulfonyloxyethoxy)ethoxy]ethoxy]ethyl]amino]-2-oxo-1-pyridyl]acetate

To a solution of (S)-methyl 2-(5-(N-(2-(2-(2-(2-hydroxyethoxy)ethoxy)ethoxy)ethyl)-1-(isoquinolin-4-yl)piperidine-3-carboxamido)-2-oxopyridin-1 (2H)-yl)acetate (200 mg, 335 μmol) in anhydrous DCM (5 mL) was added TEA (169 mg, 1.68 mmol, 233 μL) and MsCl (76.7 mg, 670 μmol, 51.9 μL) at 0° C. under nitrogen atmosphere. Then the reaction was stirred at 25° C. for 2 h under nitrogen atmosphere. On completion, the reaction mixture was neutralized with sat. NH₄Cl solution (10 mL), and extracted with DCM (20 mL×2). The residue was purified by column chromatography (SiO₂, Dichloromethane:THF=5/1 to 0/1) to give a title compound (130 mg, 49% yield) as a green gum, LC-MS (ESI⁺) m/z 675.1 (M+H)⁺.

### 16-Bromo-1-phenyl-2,5,8,11,14-pentaoxahexadecane (Intermediate HR)

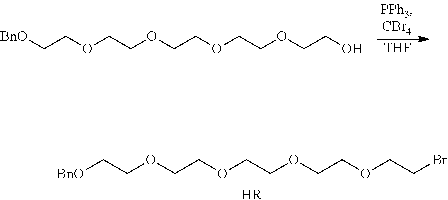

To a solution of 1-phenyl-2,5,8,11,14-pentaoxahexadecan-16-ol (4.5 g, 13.7 mmol, CAS #57671-28-0) in THF (180 mL) was added CBr₄ (9.09 g, 27.4 mmol) and PPh₃ (7.19 g, 27.4 mmol). The mixture was stirred at 0-25° C. for 5 hrs. On completion, the reaction mixture was filtered and the filtrate was concentrated in vacuo to give the crude residue. The residue was purified by column chromatography (SiO₂, Petroleum ether:Ethyl acetate=10/1 to 1/1) to give the title compound (6 g, 67% yield) as a yellow oil. ¹H NMR (400 MHz, DMSO-d₆) δ=7.39-7.23 (m, 5H), 4.49 (s, 2H), 3.75-3.69 (m, 2H), 3.58-3.54 (m, 8H), 3.52 (br d, J=2.0 Hz, 10H).

### Ethyl 2-(2-oxo-5-((1-phenyl-2,5,8,11-tetraoxatridecan-13-yl)amino)pyridin-1 (2H)-yl)acetate (Intermediate HS)

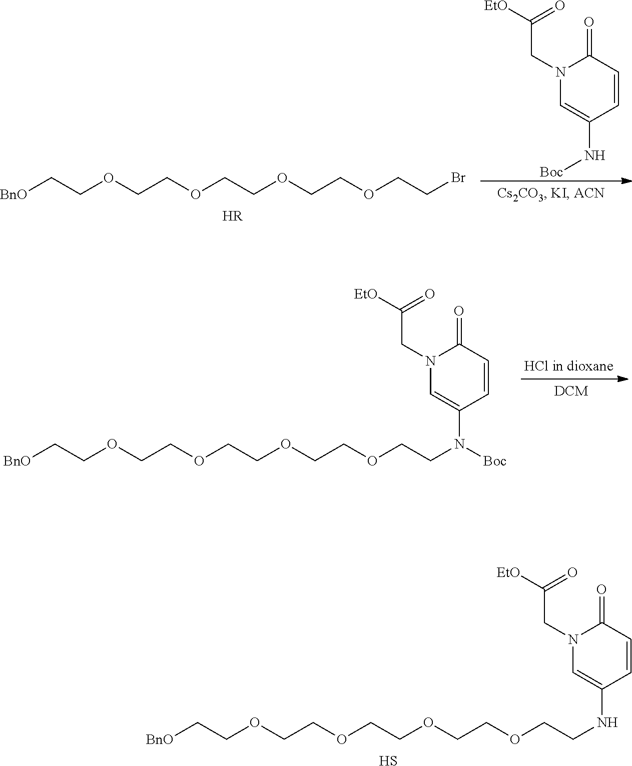

### Step 1—Ethyl 2-(2-oxo-5-((l-phenyl-2,5,8,11,14-pentaoxahexadecan-16-yl)amino)pyridin-1 (2H)-yl)acetate

To a solution of ethyl 2-(5-((tert-butoxycarbonyl)(1-phenyl-2,5,8,11,14-pentaoxahexadecan-16-yl)amino)-2-oxopyridin-1 (2H)-yl)acetate (1.8 g. 6.07 mmol, synthesized via Steps 1-2 of Intermediate HJ′), 16-bromo-1-phenyl-2,5,8,11,14-pentaoxahexadecane (4.28 g, 10.9 mmol, Intermediate HR) in ACN (100 mL) was added Cs₂CO₃ (5.94 g, 18.2 mmol) and KI (101 mg, 0.607 mmol). The mixture was then stirred at 75° C. for 12 hrs. On completion, the reaction mixture was filtered and the filtrate was concentrated in vacuo to give the crude residue. The residue was purified by column chromatography (SiO₂, Petroleum ether:Ethyl acetate=1/1 to 0/1) to give the title compound (2.2 g, 49% yield) as a yellow oil. LC-MS (ESI⁺) n/z 607.4 (M+H)⁺.

### Step 2—Ethyl 2-(2-oxo-5-((1-phenyl-2,5,8,11-tetraoxatridecan-13-yl)amino)pyridin-1 (2H)-yl)acetate

To a solution of ethyl 2-(5-((tert-butoxycarbonyl)(1-phenyl-2,5,8,11-tetraoxatridecan-13-yl)amino)-2-oxopyridin-1 (2H)-yl)acetate (2.2 g, 3.36 mmol) in DCM (10 mL) was added HCl/dioxane (1.36 mL, 8 M). The mixture was then stirred at 25° C. for 1 hr. On completion, the reaction mixture was concentrated in vacuo to give the crude residue. The residue was purified by reversed-phase HPLC (0.1% FA condition) to give a title compound (1 g, 68% yield) as a blue solid. LC-MS (ESI⁺) m/z 507.2 (M+H)⁺.

### Methyl 2-[5-[2-[2-[2-[2-(2-hydroxyethoxy)ethoxy]ethoxy]ethoxy]ethyl-[(3S)-1-(4-isoquinolyl)piperidine-3-carbonyl]amino]-2-oxo-1-pyridyl]acetate (Intermediate HT)

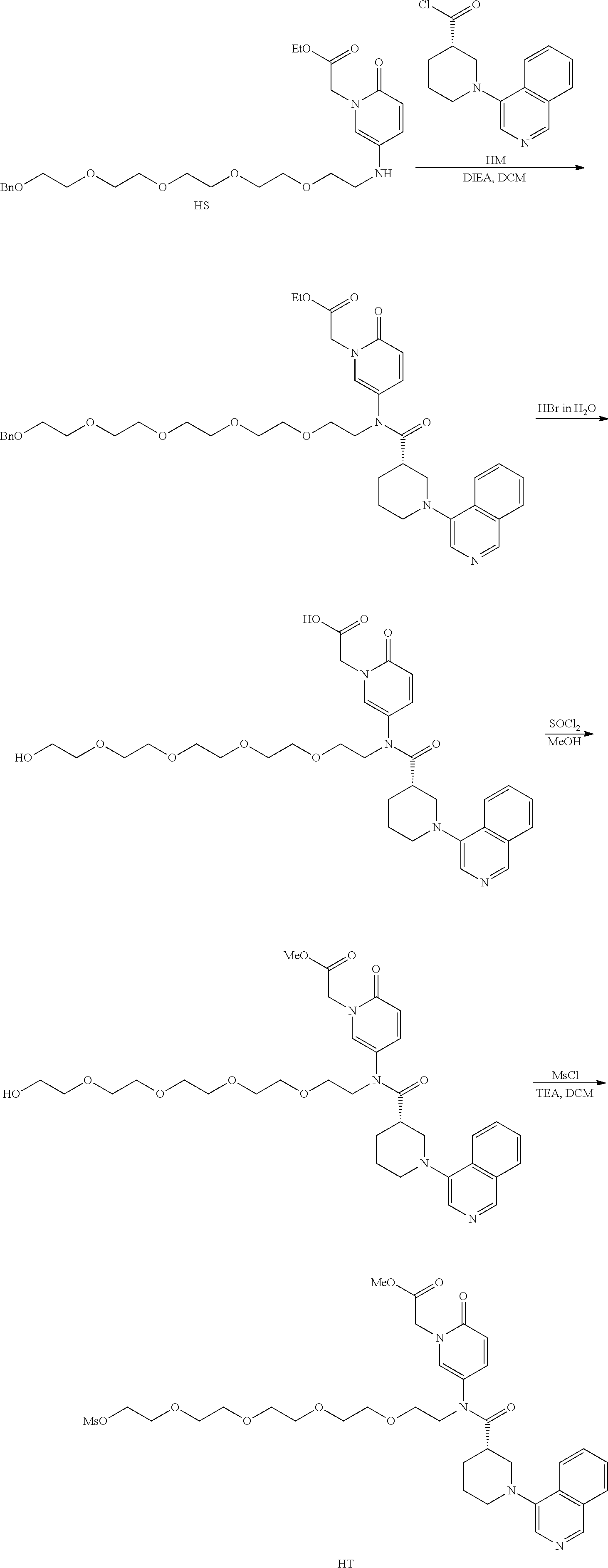

### Step 1—(S)-ethyl 2-(5-(1-(isoquinolin-4-yl)-N-(1-phenyl-2,5,8,11,14-pentaoxahexadecan-16-yl)piperidine-3-carboxamido)-2-oxopyridin-1 (2H)-yl)acetate

To a solution of ethyl 2-(2-oxo-5-((1-phenyl-2,5,8,11-tetraoxatridecan-13-yl)amino)pyridin-1 (2H)-yl)acetate (1.4 g, 2.8 mmol, Intermediate HS) in anhydrous DCM (20 mL) was added DIEA (1.79 g, 13.8 mmol, 2.41 mL) and (S)-1-(isoquinolin-4-yl)piperidine-3-carbonyl chloride (1.52 g, 5.53 mmol. Intermediate HM) in DCM (20 mL). The mixture was then stirred at 25° C. for 2 hrs. On completion, the reaction mixture was concentrated in vacuo to give the crude residue. The residue was purified by column chromatography (SiO₂, Dichloromethane:Methanol=100/1 to 10/1) to give a title compound (1 g, 21% yield) as a deep blue gum, LC-MS (ESI⁺) m/z 745.3 (M+H)⁺.

### Step 2—(S)-2-(5-(N-(14-hydroxy-3,6,9,12-tetraoxatetradecyl)-1-(isoquinolin-4-yl)piperidine-3-carboxamido)-2-oxopyridin-1 (2H)-yl)acetic acid

To a solution of (S)-ethyl 2-(5-(1-(isoquinolin-4-yl)-N-(1-phenyl-2,5,8,11,14-pentaoxahexadecan-16-yl)piperidine-3-carboxamido)-2-oxopyridin-1 (2H)-yl)acetate (1 g, 1 mmol) in HBr (2 mL, 40% solution in water) was stirred at 70° C. for 1 h under nitrogen atmosphere. On completion, the reaction mixture was filtered and the filtrate was concentrated in vacuo to give the crude residue. The residue was purified by reversed-phase HPLC (0.1% FA condition) to give a title compound (500 mg, 32% yield) as a green oil. LC-MS (ESI⁺) m/z 627.3 (M+H)⁺.

### Step 3—(S)-methyl 2-(5-(N-(14-hydroxy-3,6,9,12-tetraoxatetradecyl)-1-(isoquinolin-4-yl)piperidine-3-carboxamido)-2-oxopyridin-1 (2H)-yl)acetate

To a solution of (S)-2-(5-(N-(14-hydroxy-3,6,9,12-tetraoxatetradecyl)-1-(isoquinolin-4-yl)piperidine-3-carboxamido)-2-oxopyridin-1 (2H)-yl)acetic acid (500 mg, 799 μmol) in DCM (4 mL) was added SOCl₂ (285 mg, 2.39 mmol, 173 μL). The mixture was then stirred at 70° C. for 0.5 h. On completion, the reaction mixture was concentrated in vacuo to give the crude residue. The residue was purified by column chromatography (SiO₂. Dichloromethane:Methanol=20/1 to 10/1) to give a title compound (300 mg, 55% yield) as a yellow solid. LC-MS (ESI⁺) m/z 641.2 (M+H)⁺.

### Step 4—Methyl 2-[5-[2-[2-[2-[2-(2-hydroxyethoxy)ethoxy]ethoxy]ethoxy]ethyl-[(3S)-1-(4-isoquinolyl)piperidine-3-carbonyl]amino]-2-oxo-1-pyridyl]acetate

To a solution of (S)-methyl 2-(5-(N-(14-hydroxy-3,6,9,12-tetraoxatetradecyl)-1-(isoquinolin-4-yl)piperidine-3-carboxamido)-2-oxopyridin-1 (2H)-yl)acetate (180 mg, 281 μmol) in anhydrous DCM (5 mL) was added TEA (142 mg, 1.40 mmol, 196 μL) and MsCl (64.4 mg, 562 μmol, 43.5 L) at 0° C. under nitrogen atmosphere. Then the reaction was stirred at 25° C. for 2 hrs under nitrogen atmosphere. On completion, the reaction mixture was neutralized with sat. NH₄Cl solution (10 mL) and extracted with DCM. The residue was purified by column chromatography (SiO₂, Dichloromethane: Methanol=100/1 to 10/1) to give a title compound (170 mg, 64% yield) as a yellow oil. LC-MS (ESI⁺) m/z 718.9 (M+H)⁺.

### (((8-Bromooctyl)oxy)methyl)benzene (Intermediate HU)

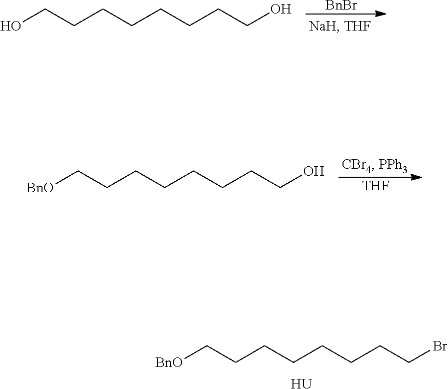

### Step 1—8-(Benzyloxy)octan-1-ol

To a suspension of NaH (3.31 g, 82.7 mmol, 60% dispersion in mineral oil) in DMF (100 mL) was slowly added a solution of octane-1,8-diol (11.0 g, 75.2 mmol, CAS #629-41-4) in DMF (50 mL) at −10° C. under nitrogen. The reaction mixture was allowed to warm to 25° C., and stirred for 1 h. Next, the reaction mixture was cooled to 0° C., and (bromomethyl) benzene (15.4 g, 90.2 mmol, 10.7 mL) was added. The reaction mixture was again warmed to 25° C., and stirred for an additional 15 h. On completion, the reaction mixture was quenched by addition of sat. NH₄Cl (60 mL) at 0° C., then 1M HCl was added to adjust the pH to 7, and the mixture was extracted with EA (200 mL×3). The combined organic layers were washed with brine (100 mL×3), dried over Na₂SO₄, filtered and concentrated under reduced pressure to give the crude product. Then the residue was purified by column chromatography (SiO₂, Petroleum ether:Ethyl acetate=10/1 to 3/1) to give the title compound (7.99 g. 45% yield) as a yellow oil. LC-MS (ESI⁺) m/z 259.2 (M+Na)⁺.

### Step 2—(((8-Bromooctyl)oxy) methyl)benzene

A solution of CBr₄ (6.68 g, 20.1 mmol) and PPh₃ (5.28 g, 20.1 mmol) in THF (50 mL) at 0° C. under N₂ atmosphere was stirred at this temperature for 0.5 h. Then 8-(benzyloxy) octan-1-ol (2.38 g, 10.0 mmol) was added dropwise at 0° C. The resulting mixture was stirred at 25° C. for 3 h. On completion, the reaction mixture was filtered and the filtrate was diluted with H₂O (50 mL) and extracted with EA (50 mL×3). The combined organic layers were dried over Na₂SO₄, filtered and concentrated under reduced pressure to give a residue. Then the residue was purified by column chromatography (SiO₂, Petroleum ether/Ethyl acetate=100/0 to 5/1) to give the title compound (2.70 g, 90% yield) as a yellow solid. ¹H NMR (400 MHz, DMSO-d₆) δ=7.35-7.29 (m, 5H), 4.44 (s, 2H), 3.53-3.50 (t, J=1.2 Hz, 2H), 3.42-3.39 (t, J=1.2 Hz, 2H), 1.80-1.76 (m, 2H), 1.53-1.51 (m, 2H), 1.30-1.26 (m, 8H).

### Ethyl 2-(5-((8-(benzyloxy)octyl)amino)-2-oxopyridin-1 (2H)-yl)acetate (Intermediate HV)

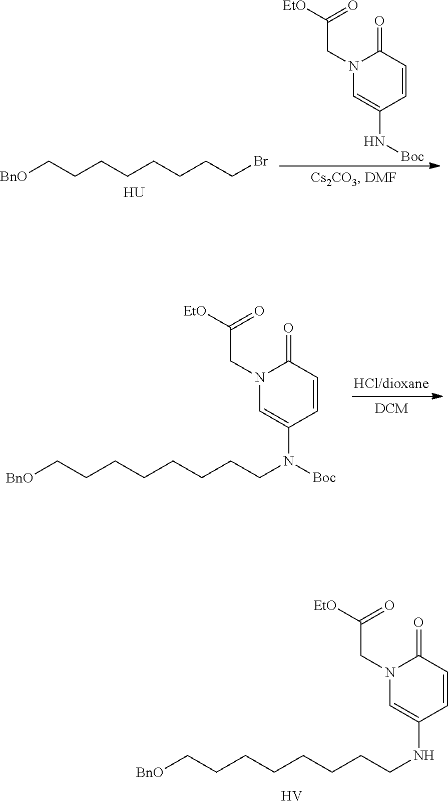

### Step 1—Ethyl 2-(5-((8-(benzyloxy) octyl) (tert-butoxycarbonyl) amino)-2-oxopyridin-1 (2H)-yl) acetate

A solution of (((8-bromooctyl)oxy) methyl)benzene (2.00 g. 6.68 mmol, Intermediate HU), ethyl 2-(5-((tert-butoxycarbonyl)amino)-2-oxopyridin-1 (2H)-yl) acetate (2.97 g, 10.0 mmol, synthesized via Steps 1-2 of Intermediate HJ′), and Cs₂CO₃ (6.53 g, 20.0 mmol) in DMF (20 mL) was stirred at 50° C. for 2 h. On completion, the reaction mixture was quenched with water (50 mL) and extracted with ethyl acetate (3×50 mL). The combined organic layers were washed with brine (100 mL) and dried over anhydrous sodium sulfate, filtered and the filtrate was concentrated in vacuo to give the crude residue. Then the residue was purified by column chromatography (SiO₂. Petroleum ether:Ethyl acetate=10/1 to 1/1) to give the title compound (1.3 g) as a brown oil. LC-MS (ESI⁺) m/z 515.3 (M+H)⁺.

### Step 2—Ethyl 2-(5-((8-(benzyloxy)octyl)amino)-2-oxopyridin-1 (2H)-yl)acetate

A solution of ethyl 2-(5-((8-(benzyloxy)octyl)(tert-butoxycarbonyl)amino)-2-oxopyridin-1(2H)-yl) acetate (1.60 g, 3.11 mmol) in HCl/dioxane (14 mL) and DCM (6 mL) was stirred at 25° C. for 2 h. On completion, the mixture was concentrated in vacuo to give the residue. Then the residue was purified by reversed-phase HPLC (0.8 g/L ammonium bicarbonate) to give the title compound (1.00 g, 73% yield) as a green oil. LC-MS (ESI⁺) m/z 415.2 (M+H)⁺.

### Methyl (S)-2-(5-(1-(isoquinolin-4-yl)-N-(8-oxooctyl)piperidine-3-carboxamido)-2-oxopyridin-1 (2H)-yl)acetate (Intermediate HW)

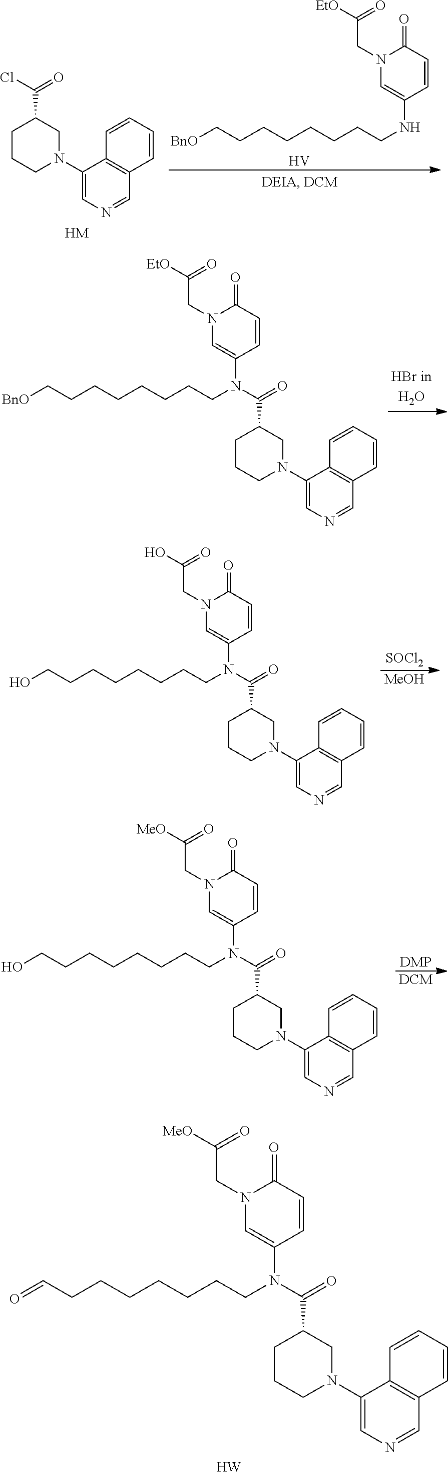

### Step 1—Ethyl (S)-2-(5-(N-(8-(benzyloxy)octyl)-1-(isoquinolin-4-yl)piperidine-3-carboxamido)-2-oxopyridin-1 (2H)-yl)acetate

A solution of (S)-1-(isoquinolin-4-yl)piperidine-3-carbonyl chloride (1.06 g. 3.86 mmol, Intermediate HM), ethyl 2-[5-(8-benzyloxyoctylamino)-2-oxo-1-pyridyl]acetate (1.00 g, 2.41 mmol, Intermediate HV), and DIEA (1.56 g, 12.0 mmol, 2.10 mL) in DCM (10 mL) was stirred at 25° C. for 2 h. On completion, the reaction mixture was quenched with water (10 mL) and extracted with dichloromethane (3×10 mL). The combined organic layers were washed with brine (10 mL) and dried over anhydrous sodium sulfate, filtered and the filtrate was concentrated in vacuo to give the crude residue. The crude residue was purified by reversed-phase HPLC (0.8 g/L ammonium bicarbonate) to give the tittle compound (600 mg, 37% yield) as a blue oil. LC-MS (ESI⁺) m/z 653.2 (M+H)⁺.

### Step 2—(S)-2-(5-(N-(8-hydroxyoctyl)-1-(isoquinolin-4-yl)piperidine-3-carboxamido)-2-oxopyridin-1 (2H)-yl)acetic acid

A solution of ethyl (S)-2-(5-(N-(8-(benzyloxy)octyl)-1-(isoquinolin-4-yl)piperidine-3-carboxamido)-2-oxopyridin-1 (2H)-yl)acetate (600 mg, 919 μmol) in HBr (10 mL) was stirred at 65° C. for 2 h. On completion, the reaction mixture was added Et₃N until the pH 7, then diluted with water (10×3 mL) and extracted with dichloromethane (3×10 mL). The combined water layer was concentrated in vacuo to give the tittle compound (15.0 g) as a green solid. LC-MS (ESI⁻) m/z 535.2 (M+H)⁺.

### Step 3—Methyl (S)-2-(5-(N-(8-hydroxyoctyl)-1-(isoquinolin-4-yl)piperidine-3-carboxamido)-2-oxopyridin-1 (2H)-yl)acetate

A solution of 2-[5-[8-hydroxyoctyl-[(3S)-1-(4-isoquinolyl)piperidine-3-carbonyl]amino]-2-oxo-1-pyridyl]acetic acid (15 g, 23.6 mmol), SOCl₂ (8.42 g, 70.7 mmol. 5.14 mL) in MeOH (50 mL) was stirred at 70° C. for 10 min. On completion, sat. NaHCO₃ was added to the reaction mixture until the pH=7, then the mixture was diluted with water (30 mL) and extracted with ethyl acetate (3×50 mL). The combined organic layers were dried over anhydrous sodium sulfate, filtered and the filtrate was concentrated in vacuo to give the tittle compound (340 mg) as a green solid. LC-MS (ESI⁺) m/z 549.2 (M+H)⁺.

### Step 4—Methyl (S)-2-(5-(1-(isoquinolin-4-yl)-N-(8-oxooctyl)piperidine-3-carboxamido)-2-oxopyridin-1 (2H)-yl)acetate

A solution of methyl (S)-2-(5-(N-(8-hydroxyoctyl)-1-(isoquinolin-4-yl)piperidine-3-carboxamido)-2-oxopyridin-1 (2H)-yl)acetate (280 mg, 510 μmol) and DMP (324 mg, 765 μmol, 237 μL) in DCM (3 mL) was stirred at 0° C. for 2 h. On completion, sat. NaHCO₃ was added to the reaction mixture until the pH=7, then diluted with water (10 mL) and extracted with ethyl acetate (3×10 mL). The combined organic layers were dried over anhydrous sodium sulfate, filtered and the filtrate was concentrated in vacuo to give the title compound (300 mg) as a yellow solid. LC-MS (ESI⁺) m/z 547.2 (M+H)⁺.

### 11-Bromoundecoxymethylbenzene (Intermediate HX)

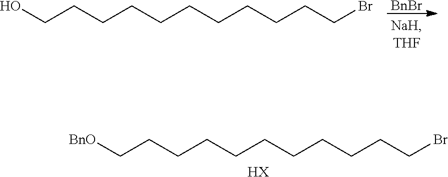

To a solution of 11-bromoundecan-1-ol (10 g, 40 mmol, CAS #1611-56-9) in THF (100 mL) was added NaH (1.59 g, 39.8 mmol, 60% dispersion in mineral oil) at 0° C. After addition, the mixture was stirred at this temperature for 10 mins, and then BnBr (6.81 g, 39.8 mmol, 4.73 mL) was added dropwise at 0° C. The resulting mixture was then stirred at 25° C. for 16 h. The reaction mixture was quenched by addition of NH₄Cl (20 mL) at 0° C., and then diluted with water (100 mL) and extracted with EA (100 mL×3). The combined organic layers were washed with brine (100 mL×2), dried over Na₂SO₄, filtered and concentrated under reduced pressure to give a residue. The residue was purified by column chromatography (SiO₂, Petroleum ether:Ethyl acetate=1/0 to 10/1) to give the title compound (8.7 g, 64% yield) as a colorless oil. ¹H NMR (400 MHz. CHLOROFORM-d) δ=1.18-1.31 (m, 14H), 1.52-1.58 (m, 2H), 1.79 (q, J=7.2 Hz, 2H), 3.34 (t, J=6.8 Hz. 2H), 3.40 (t, J=6.4 Hz, 2H), 4.44 (s, 2H), 7.19-7.30 (m, 5H).

### Ethyl 2-[5-(11-benzyloxyundecylamino)-2-oxo-1-pyridyl]acetate (Intermediate HY)

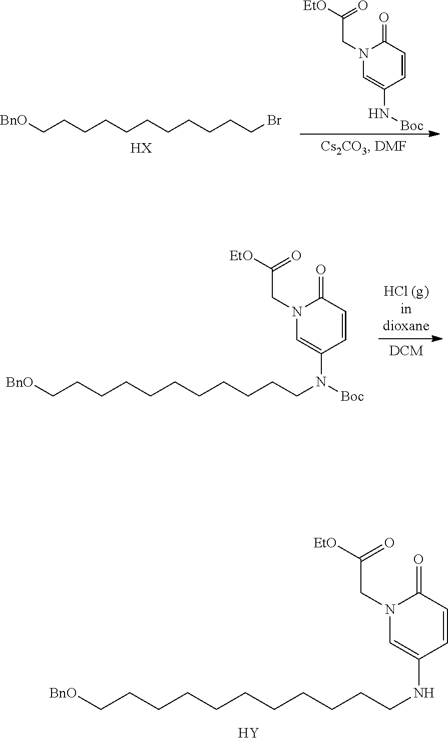

### Step 1—Ethyl 2-[5-[11-benzyloxyundecyl(tert-butoxycarbonyl)amino]-2-oxo-1-pyridyl]acetate

To a solution of 11-bromoundecoxymethylbenzene (3 g, 9 mmol, Intermediate HX) in ACN (50 mL) was added Cs₂CO₃ (8.59 g, 26.4 mmol) and ethyl 2-[5-(tert-butoxycarbonylamino)-2-oxo-1-pyridyl]acetate (2.60 g, 8.79 mmol, synthesized via Steps 1-2 of Intermediate HJ′). The mixture was then stirred at 50° C. for 12 h. On completion, the reaction mixture was filtered and the filtrate was concentrated in vacuo to give the crude residue. The residue was purified by column chromatography (SiO₂, Petroleum ether:Ethyl acetate=1/0 to 8/1) to give the title compound (3 g, 58% yield) as a brown oil. LC-MS (ESI⁺) m/z 557.2 (M+H)⁺.

### Step 2—Ethyl 2-[5-(11-benzyloxyundecylamino)-2-oxo-1-pyridyl]acetate

To a solution of ethyl 2-[5-[11-benzyloxyundecyl(tert-butoxycarbonyl)amino]-2-oxo-1-pyridyl]acetate (2.9 g. 5.21 mmol) in DCM (29 mL) was added HCl/dioxane (6 M. 5.80 mL), then the mixture was stirred at 25° C. for 1 h. On completion, sat. sodium bicarbonate aqueous solution was added to the reaction mixture until the pH˜7, then diluted with water (100 mL) and extracted with ethyl acetate (150×3 mL). The combined organic layers were dried over anhydrous sodium sulfate, filtered and the filtrate was concentrated in vacuo to give the title compound (2.37 g) as a blue solid. LC-MS (ESI⁺) m/z 457.2 (M+H)⁺.

### Methyl 2-[5-[[(3S)-1-(4-isoquinolyl) piperidine-3-carbonyl]-(11-oxoundecyl) amino]-2-oxo-1-pyridyl]acetate (Intermediate HZ)

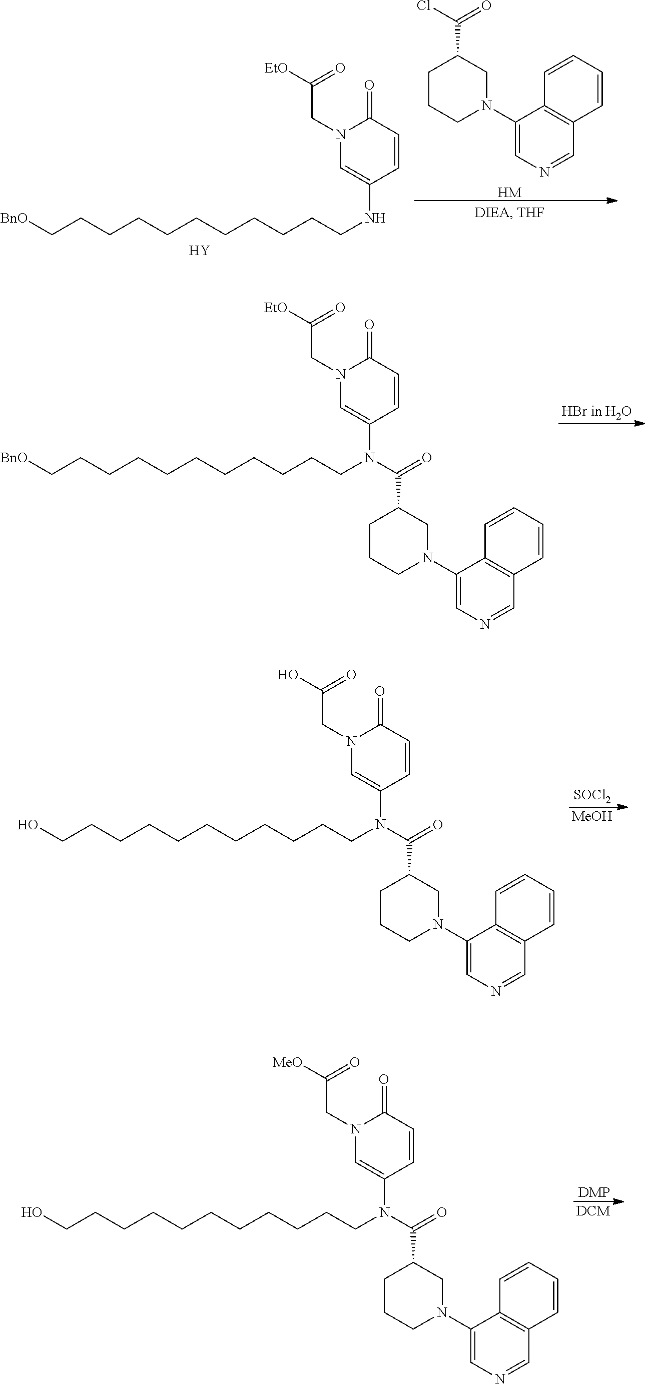

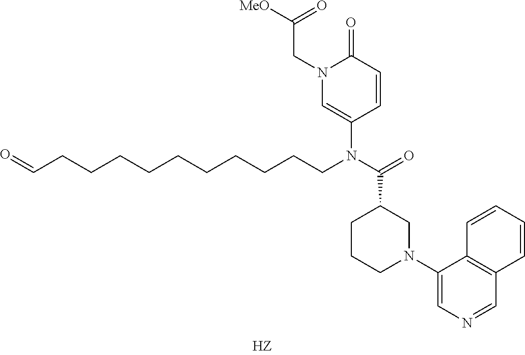 [SMILES: COC(=O)Cn1cc(N(CCCCCCCCCCC=O)C(=O)[C@H]2CCCN(c3cncc4ccccc34)C2)ccc1=O]

### Step 1—Ethyl 2-[5-[11-benzyloxyundecyl-[(3S)-1-(4-isoquinolyl) piperidine-3-carbonyl]amino]-2-oxo-1-pyridyl]acetate

To a mixture of ethyl 2-[5-(11-benzyloxyundecylamino)-2-oxo-1-pyridyl]acetate (1 g, 2.19 mmol) and DIEA (849 mg, 6.57 mmol, 1.14 mL, Intermediate HY) in DCM (20 mL) was added (3S)-1-(4-isoquinolyl)piperidine-3-carbonyl chloride (903 mg, 3.29 mmol, Intermediate HM) and the mixture was degassed and purged with N₂ three times. Then the mixture was stirred at 0-25° C. for 2 h under N₂ atmosphere. On completion, the mixture was concentrated in vacuo. The crude residue was purified by reversed-phase HPLC (neutral condition) to give the title compound (1 g, 66% yield) as a white solid. LC-MS (ESI⁺) m/z 695.4 (M+H)⁺.

### Step 2—2-[5-[l1-Hydroxyundecyl-[(3S)-1-(4-isoquinolyl) piperidine-3-carbonyl]amino]-2-oxo-1-pyridyl]acetic acid

To a solution of ethyl 2-[5-[11-benzyloxyundecyl-[(3S)-1-(4-isoquinolyl)piperidine-3-carbonyl]amino]-2-oxo-1-pyridyl]acetate (1 g, 1 mmol) was added HBr (14.9 g, 73.7 mmol, 18.4 mL, 40% solution). The mixture was then stirred at 60° C. for 2 h. On completion, the reaction mixture was added TEA until the pH-7, then diluted with water (200 mL) and extracted with dichloromethane (3×300 mL). The combined organic layers were dried over anhydrous sodium sulfate, filtered and the filtrate was concentrated in vacuo to give the title compound (1.6 g) as green oil. LC-MS (ESI⁺) m/z 577.4 (M+H)⁺.

### Step 3—Methyl 2-[5-[11-hydroxyundecyl-[(3S)-1-(4-isoquinolyl) piperidine-3-carbonyl]amino]-2-oxo-1-pyridyl]acetate

To a solution of 2-[5-[11-hydroxyundecyl-[(3S)-1-(4-isoquinolyl) piperidine-3-carbonyl]amino]-2-oxo-1-pyridyl]acetic acid (1.5 g, 2.6 mmol) in MeOH (15 mL) was added SOCl₂ (464 mg, 3.90 mmol, 283 μL). The mixture was then stirred at 70° C. for 1 h. On completion, the reaction mixture was added sat. NaHCO₃ solution until the pH=8, then diluted with water (300 mL) and extracted with ethyl acetate (3×500 mL). The combined organic layers were dried over anhydrous sodium sulfate, filtered and the filtrate was concentrated in vacuo to give the crude residue. The residue was purified by column chromatography (SiO₂, Dichloromethane:Methanol=1/0 to 10/1) to give the title compound (520 mg, 29% yield) as brown oil. LC-MS (ESI⁺) m/z 591.3 (M+H)⁺.

### Step 4—Methyl 2-[5-[[(3S)-1-(4-isoquinolyl) piperidine-3-carbonyl]-(11-oxoundecyl) amino]-2-oxo-1-pyridyl]acetate

To a solution of methyl 2-[5-[11-hydroxyundecyl-[(3S)-1-(4-isoquinolyl)piperidine-3-carbonyl]amino]-2-oxo-1-pyridyl]acetate (500 mg, 846 μmol) in DCM (5 mL) was added DMP (538 mg, 1.27 mmol, 393 μL) at 0° C. The mixture was then stirred at 0-25° C. for 1 h. On completion, the reaction mixture was added sodium bicarbonate saturated solution until the pH=8, then diluted with water (100 mL) and extracted with dichloromethane (200×3 mL). The combined organic layers were dried over anhydrous sodium sulfate, filtered and the filtrate was concentrated in vacuo to give the title compound (540 mg) as a white solid. LC-MS (ESI⁺) m/z 589.2 (M+H)⁺.

### (((14-Bromotetradecyl)oxy)methyl)benzene (Intermediate IA)

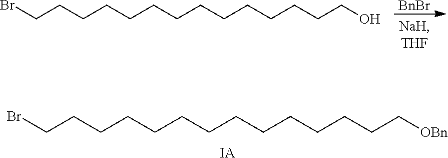

To a solution of 14-bromotetradecan-1-ol (10 g, 30 mmol, CAS #72995-94-9) in THF (100 mL) was added NaH (1.36 g, 34.1 mmol, 60% dispersion in mineral oil) at 0° C. After addition, the mixture was stirred at this temperature for 10 min, and then BnBr (5.83 g, 34.1 mmol) was added dropwise at 0° C. The resulting mixture was stirred at 0° C. for 30 min. On completion, the reaction mixture was quenched by addition of NH₄Cl (20 mL) at 0° C., and then diluted with H₂O (100) mL and extracted with EA (80 mL×3). The combined organic layers were washed with brine (100 mL×2), dried over anhydrous sodium sulfate, filtered and concentrated under reduced pressure to give a residue. The residue was purified by column chromatography (SiO₂, Petroleum ether/Ethyl acetate=1/0 to 10/1) to give a title compound (10 g, 76% yield) as a colorless oil. ¹H NMR (400 MHz, DMSO-d₆) δ=7.36-7.24 (m, 5H), 4.43 (s, 2H), 4.29 (t, J=5.2 Hz, 1H), 3.52-3.48 (m, 3H), 3.42-3.39 (m, 2H), 3.39-3.34 (m, 2H), 1.78 (quin. J=6.8 Hz, 4H), 1.55-1.49 (m, 2H), 1.37 (br dd, J=6.8, 13.2 Hz, 4H), 1.24 (br s, 10H).

### Ethyl 2-(5-((14-(benzyloxy)tetradecyl)(tert-butoxycarbonyl)amino)-2-oxopyridin-1 (2H)-yl)acetate (Intermediate IB)

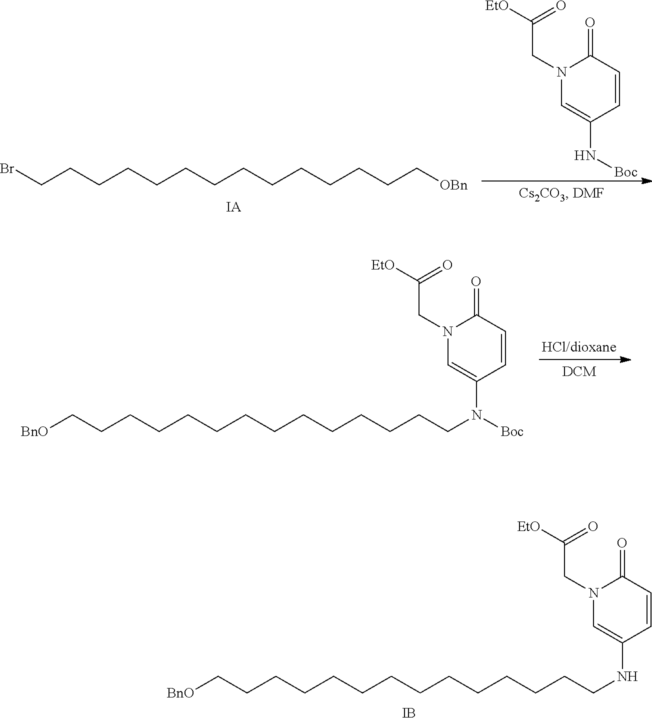

### Step 1—Ethyl 2-(5-((14-(benzyloxy)tetradecyl)(tert-butoxycarbonyl)amino)-2-oxopyridin-1 (2H)-yl)acetate

To a solution of (((14-bromotetradecyl)oxy)methyl)benzene (3 g, 7.82 mmol, Intermediate IA) and ethyl 2-(5-((tert-butoxycarbonyl)amino)-2-oxopyridin-1 (2H)-yl)acetate (2.32 g, 7.82 mmol, synthesized via Steps 1-2 of Intermediate HJ′) in ACN (50 mL) was added Cs₂CO₃ (7.65 g, 23.4 mmol). The mixture was then stirred at 50° C. for 6 h. On completion, the reaction mixture was filtered and the filtrate concentrated under reduced pressure to give a residue. The residue was purified by column chromatography (SiO₂, Petroleum ether/Ethyl acetate=1/0 to 1/1) to give the title compound (1.55 g, 33% yield) as a yellow oil. LC-MS (ESI⁺) m/z 599.3 (M+H)⁺.

### Step 2—Ethyl 2-(5-((14-(benzyloxy)tetradecyl)(tert-butoxycarbonyl)amino)-2-oxopyridin-1 (2H)-yl)acetate

To a solution of ethyl 2-(5-((14-(benzyloxy)tetradecyl)(tert-butoxycarbonyl)amino)-2-oxopyridin-1 (2H)-yl)acetate (1.55 g, 2.59 mmol) in DCM (5 mL) was added HCl/dioxane (8 M, 5 mL). The mixture was then stirred at 25° C. for 2 h. On completion, the reaction mixture was concentrated under reduced pressure to give a residue. The residue was purified by prep-HPLC (neutral condition) to give the title compound (560 mg, 43% yield) as a blue solid. LC-MS (ESI⁺) m/z 499.3 (M+H)⁻.

### Methyl (S)-2-(5-(1-(isoquinolin-4-yl)-N-(14-oxotetradecyl)piperidine-3-carboxamido)-2-oxopyridin-1 (2H)-yl)acetate (Intermediate IC)

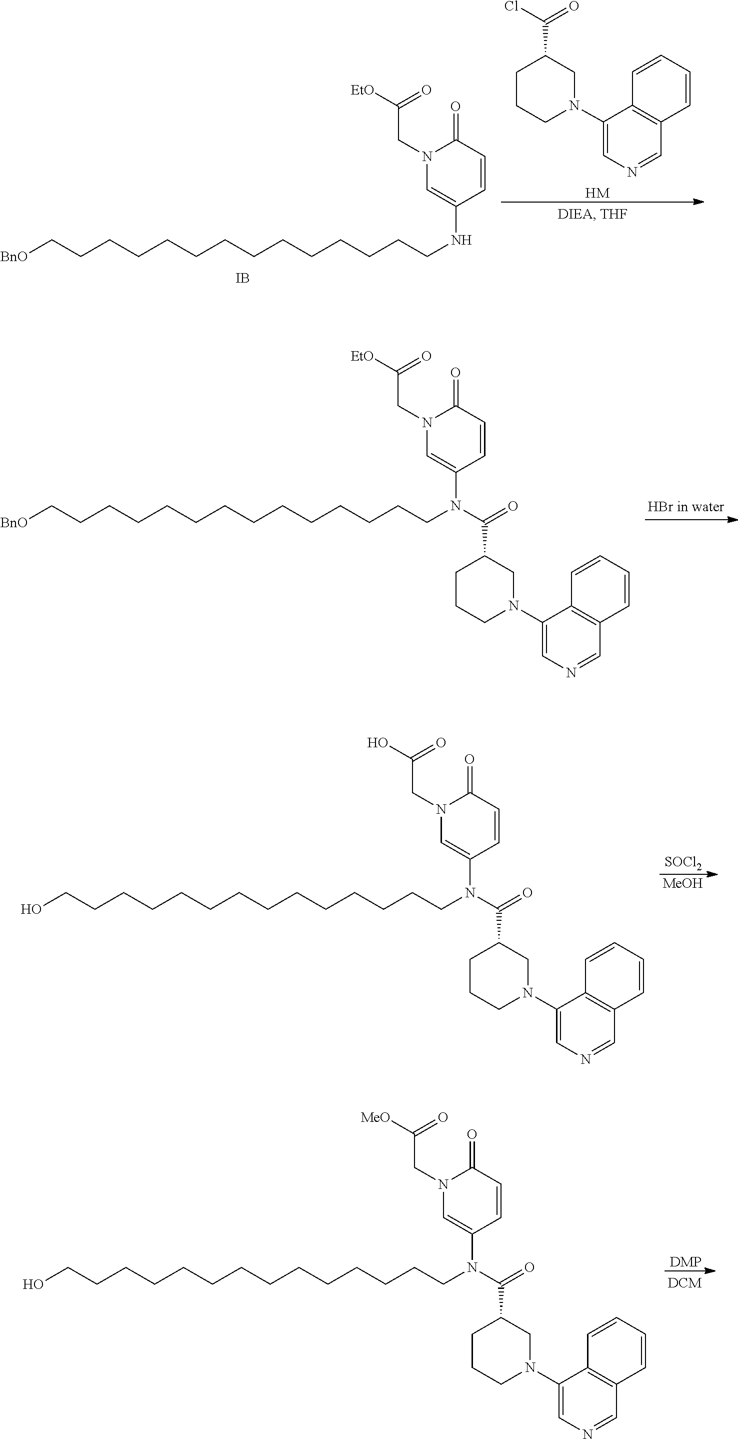

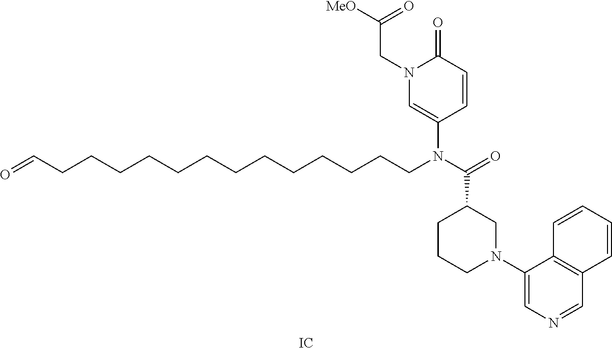 [SMILES: COC(=O)Cn1cc(N(CCCCCCCCCCCCCC=O)C(=O)[C@H]2CCCN(c3cncc4ccccc34)C2)ccc1=O ; "IC"]

### Step 1—Ethyl (S)-2-(5-(N-(14-(benzyloxy)tetradecyl)-1-(isoquinolin-4-yl)piperidine-3-carboxamido)-2-oxopyridin-1 (2H)-yl)acetate

To a solution of ethyl 2-(5-((14-(benzyloxy)tetradecyl)amino)-2-oxopyridin-1 (2H)-yl)acetate (510 mg, 1.02 mmol, Intermediate IB) and (S)-1-(isoquinolin-4-yl)piperidine-3-carbonyl chloride (337 mg, 1.23 mmol, Intermediate HM) in THF (6 mL) was added DIEA (660 mg, 5.11 mmol) at 0° C. The mixture was then stirred at 25° C. for 1 h. On completion, the reaction mixture was concentrated under reduced pressure to give a residue. The residue was purified by prep-HPLC (HCl condition) to give a title compound (392 mg, 49% yield, HCl) as a green solid. LC-MS (ESI⁺) m/z 737.6 (M+H)⁺.

### Step 2—(S)-2-(5-(N-(14-hydroxytetradecyl)-1-(isoquinolin-4-yl)piperidine-3-carboxamido)-2-oxopyridin-1(2H)-yl)acetic acid

A solution of ethyl (S)-2-(5-(N-(14-(benzyloxy)tetradecyl)-1-(isoquinolin-4-yl)piperidine-3-carboxamido)-2-oxopyridin-1 (2H)-yl)acetate (392 mg, 531 μmol) in HBr (4 mL) was stirred at 60° C. for 1 h. On completion, the reaction mixture was concentrated under reduced pressure to give a residue. The residue was purified by prep-HPLC (neutral condition) to give a title compound (84 mg, 23% yield) as a yellow solid. LC-MS (ESI⁺) m/z 619.4 (M+H)⁺.

### Step 3—(S)-methyl 2-(5-(N-(14-hydroxytetradecyl)-1-(isoquinolin-4-yl)piperidine-3-carboxamido)-2-oxopyridin-1 (2H)-yl)acetate

To a solution of (S)-2-(5-(N-(14-hydroxytetradecyl)-1-(isoquinolin-4-yl)piperidine-3-carboxamido)-2-oxopyridin-1 (2H)-yl)acetic acid (84 mg, 135 μmol) in MeOH (2 mL) was added SOCl₂ (16.1 mg, 135 μmol). The mixture was then stirred at 70° C. for 1 h. On completion, the reaction mixture was concentrated under reduced pressure to give a residue. The residue was purified by column chromatography (SiO₂, Petroleum ether/Ethyl acetate=1/0 to 0/1, DCM:MeOH=10:1) to give a title compound (55 mg, 64% yield) as a white solid. LC-MS (ESI⁺) m/z 633.4 (M+H)⁺.

### Step 4—Methyl (S)-2-(5-(1-(isoquinolin-4-yl)-N-(14-oxotetradecyl)piperidine-3-carboxamido)-2-oxopyridin-1 (2H)-yl)acetate

To a solution of methyl (S)-2-(5-(N-(14-hydroxytetradecyl)-1-(isoquinolin-4-yl)piperidine-3-carboxamido)-2-oxopyridin-1 (2H)-yl)acetate (50 mg, 79 μmol) in DCM (2 mL) was added DMP (67.0 mg, 158 μmol) at 0° C. The mixture was then stirred at 0-25° C. for 1 h. On completion, the reaction mixture was quenched with addition of Na₂SO₃ solution (6 mL), then diluted with H₂O (5 mL) and extracted with EA (10 mL×2). The combined organic layers were washed with NaHCO₃ solution (3 mL), then concentrated under reduced pressure to give the title compound (45 mg) as a yellow solid. LC-MS (ESI⁺) m/z 631.4 (M+H)⁺

### Tert-butyl 4-(4-chloro-2,5-difluoro-phenyl)-33-difluoro-piperidine-1-carboxylate (Intermediate MG)

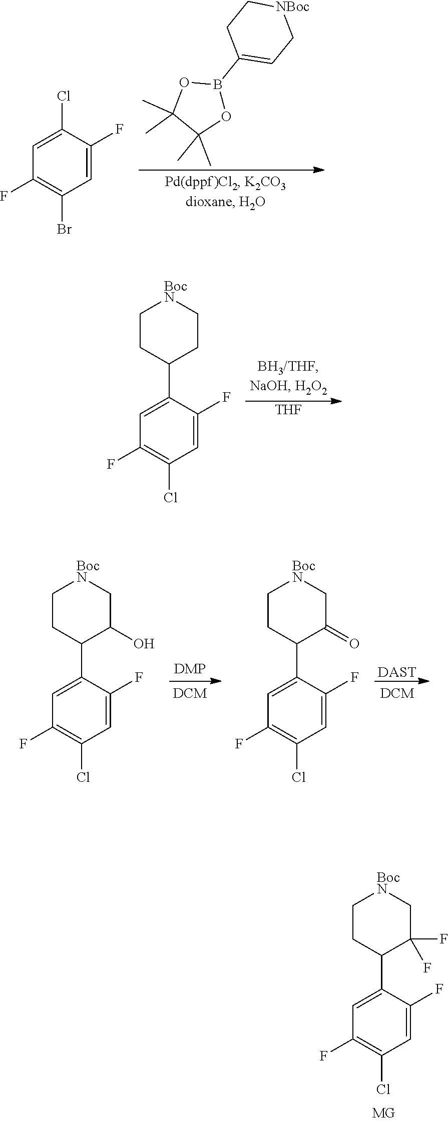

### Step 1—Tert-butyl 4-(4-chloro-2,5-difluoro-phenyl)-3,6-dihydro-2H-pyridine-1-carboxylate

A mixture of 1-bromo-4-chloro-2,5-difluoro-benzene (10 g. 44 mmol. CAS #17291-33-4), tert-butyl 4-(4,4,5,5-tetramethyl-1,3,2-dioxaborolan-2-yl)-3,6-dihydro-2H-pyridine-1-carboxylate (13.6 g, 44.0 mmol, CAS #286961-14-6), Pd(dppf)Cl₂ (3.22 g, 4.40 mmol), K₂CO₃ (18.2 g, 132 mmol) in dioxane (100 mL) and water (20 mL) was degassed and purged with N₂ three times. Then the mixture was stirred at 80° C. for 2 hrs under N₂ atmosphere. The mixture was concentrated in vacuo. The residue was purified by column chromatography (SiO₂, Petroleum ether:Ethyl acetate=1/0 to 10/1) to give the title compound (10.9 g, 68% yield) as a white solid. LC-MS (ESI⁺) m/z 273.9 (M−56)⁺; ¹H NMR (400 MHz, DMSO-d6) δ=7.62 (dd, J=6.4, 10.4 Hz, 1H), 7.46 (dd, J=6.8, 10.0 Hz, 1H), 6.09 (s, 1H), 3.99 (s, 2H), 3.51 (t, 0.1=5.6 Hz, 2H), 2.41 (s, 2H), 1.42 (s, 9H).

### Step 2—Tert-butyl 4-(4-chloro-2,5-difluoro-phenyl)-3-hydroxy-piperidine-1-carboxylate

A mixture of tert-butyl 4-(4-chloro-2,5-difluoro-phenyl)-3,6-dihydro-2H-pyridine-1-carboxylate (4 g, 10 mmol) in THF (40 mL) was added BH₃·THF (1 M, 18.19 mL) added 0° C. The mixture was stirred at 25° C. for 16 hrs. Then NaOH (1.21 g, 30.3 mmol) in water (8 mL) was added dropwise at 0° C., and the mixture was stirred for 0.5 hr. Then, H₂O₂ (3.44 g, 30.3 mmol, 2.91 mL, 30% solution) was added, and the mixture was stirred at 50° C. for 2.5 hrs under N₂ atmosphere. On completion, the reaction mixture was quenched with Na₂S₂O₃ (100 mL) and extracted with ethyl acetate (2×100 mL). The combined organic layers were washed with brine (100 mL) and dried over anhydrous sodium sulfate, filtered and the filtrate was concentrated in vacuo to give the crude residue. The residue was purified by column chromatography (SiO₂, Petroleum ether:Ethyl acetate=1/0 to 4/1) to give the title compound (2.3 g, 53% yield) as a white solid. LC-MS (ESI⁺) m/z 291.9 (M−55)⁺; ¹H NMR (400 MHz, CHLOROFORM-d) δ=7.15 (dd, J=6.4, 9.2 Hz, 1H), 7.06 (dd. J=6.4, 9.2 Hz, 1H), 4.41 (br d, J=4.0 Hz, 1H), 4.27-4.09 (in, 1H), 3.75 (dt, J=4.8, 10.0 Hz, 1H), 2.94-2.86 (m, 1H), 2.77 (br t, J=12.0 Hz, 1H), 2.64 (br t, J=12.0 Hz, 1H), 1.85-1.79 (m, 1H), 1.72-1.64 (m, 2H), 1.49 (s, 9H).

### Step 3—Tert-butyl 4-(4-chloro-2,5-difluoro-phenyl)-3-oxo-piperidine-1-carboxylate

To a solution of tert-butyl 4-(4-chloro-2,5-difluoro-phenyl)-3-hydroxy-piperidine-1-carboxylate (2.3 g. 6.6 mmol) in DCM (20 mL) was added DMP (4.21 g, 9.92 mmol, 3.07 mL). The mixture was then stirred at 0-25° C. for 3 hrs. On completion, the reaction mixture was quenched with Na₂S₂O₃ (40 mL) and extracted with ethyl acetate (100×2 mL). The combined organic layers were washed with brine (100 mL) and dried over anhydrous sodium sulfate, filtered and the filtrate was concentrated in vacuo to give the crude residue. The crude residue was purified by reversed-phase HPLC (0.1% FA condition) to give the title compound (1.9 g, 80% yield) as a white solid. LC-MS (ESI⁺) m/z 290. (M−55)⁺.

### Step 4—Tert-butyl 4-(4-chloro-2,5-difluoro-phenyl)-3,3-difluoro-piperidine-1-carboxylate

A mixture of tert-butyl 4-(4-chloro-2,5-difluoro-phenyl)-3-oxo-piperidine-1-carboxylate (1.8 g, 5.2 mmol) in DCM (18 mL) was degassed and purged with N₂ three times. Then was added DAST (1.26 g, 7.81 mmol, 1.03 mL) and the mixture was stirred at 25° C. for 12 hrs under N₂ atmosphere. On completion, the reaction mixture was quenched with saturated sodium bicarbonate aqueous solution (100 mL) and extracted by dichloromethane (100×2 mL). The combined organic layers were washed with brine (100 mL) and dried over anhydrous sodium sulfate, filtered and the filtrate was concentrated in vacuo to give the crude residue. The residue was purified by column chromatography (SiO₂, Petroleum ether:Ethyl acetate=1/0 to 10/1) to give the title compound (0.9 g, 46% yield) as a white solid. LC-MS (ESI⁺) m/z 311.8 (M−55)⁺.

### Tert-butyl (4R)-4-[4-[2-(dimethylcarbamoyl)-7-fluoro-6-[1-(3-pyrazol-1-ylpropanoyl)-3,6-dihydro-2H-pyridin-5-yl]-1H-indol-4-yl]-2,5-difluoro-phenyl]-3,3-difluoro-piperidine-1-carboxylate (Intermediate MH) & tert-butyl (4S)-4-[4-[2-(dimethylcarbamoyl)-7-fluoro-6-[1-(3-pyrazol-1-ylpropanoyl)-3,6-dihydro-2H-pyridin-5-yl]-1H-indol-4-yl]-2,5-difluoro-phenyl]-3,3-difluoro-piperidine-1-carboxylate (Intermediate MI)

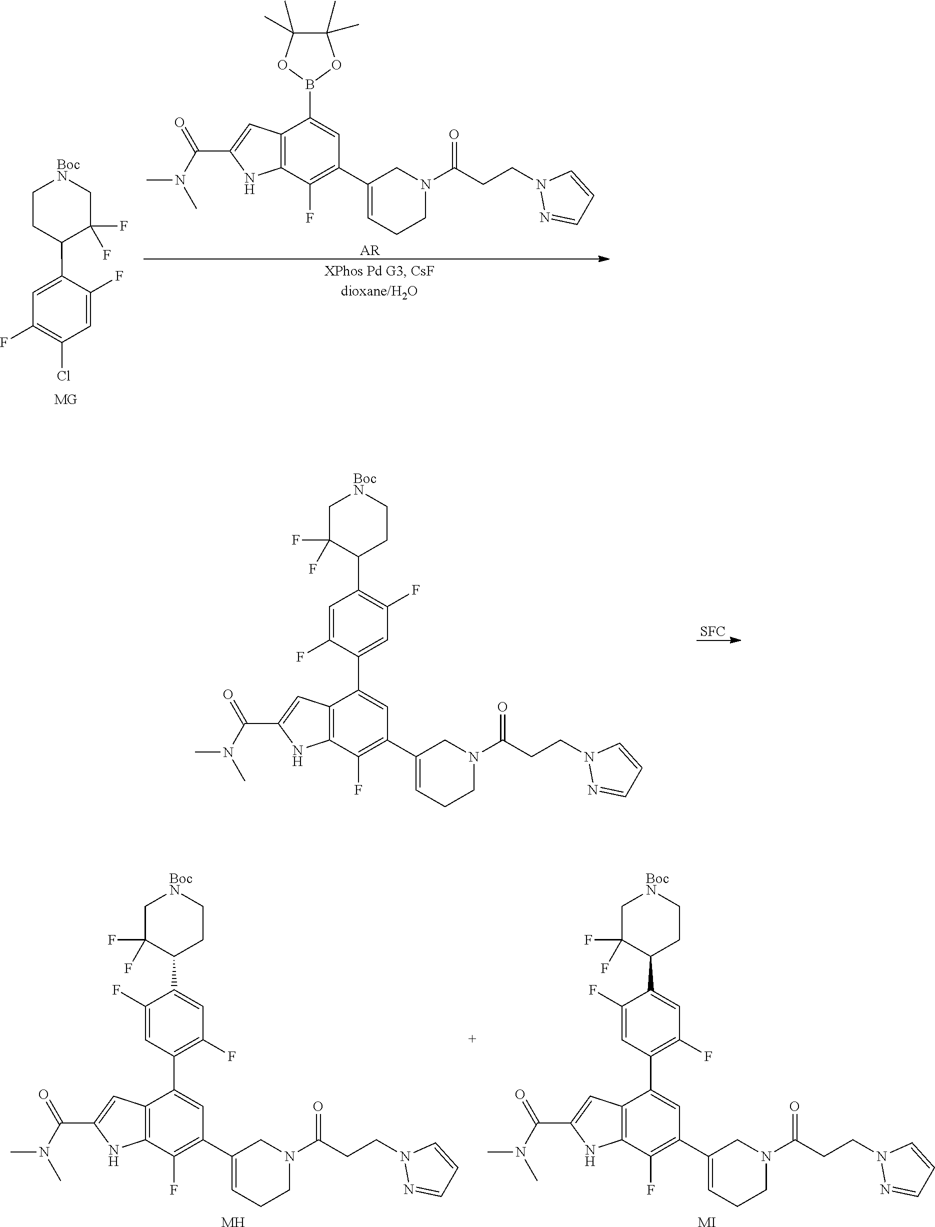

### Step 1—Tert-butyl 4-[4-[2-(dimethylcarbamoyl)-7-fluoro-6-[1-(3-pyrazol-1-ylpropanoyl)-3,6-dihydro-2H-pyridin-5-yl]-1H-indol-4-yl]-2,5-difluoro-phenyl]-3,3-difluoro-piperidine-1-carboxylate

A mixture of tert-butyl 4-(4-chloro-2,5-difluoro-phenyl)-3,3-difluoro-piperidine-1-carboxylate (800 mg, 2.18 mmol, Intermediate MG), 7-fluoro-N,N-dimethyl-6-[1-(3-pyrazol-1-ylpropanoyl)-3,6-dihydro-2H-pyridin-5-yl]-4-(4,4,5,5-tetramethyl-1,3,2-dioxaborolan-2-yl)-1H-indole-2-carboxamide (1.16 g, 2.18 mmol, Intermediate AR), XPhos Pd G₃ (184 mg, 218 μmol), and CsF (826 mg, 5.44 mmol) in dioxane (8 mL) and water (2 mL) was degassed and purged with N₂ three times. Then the mixture was stirred at 80° C. for 2 hrs under N₂ atmosphere. The mixture was concentrated in vacuo. The residue was purified by column chromatography (SiO₂, Tetrahydrofuran:Ethyl acetate=0/1 to 1/3) to give the title compound (1.4 g, 86% yield) as a white solid. LC-MS (ESI⁺) m/z 741.8 (M+H)⁺; ¹H NMR (400 MHz, CHLOROFORM-d) δ=9.58 (br d, J=12.4 Hz, 1H), 7.56-7.48 (m, 2H), 7.24-7.19 (m, 1H), 7.00 (dd, J=6.0, 13.2 Hz, 1H), 6.76 (br s, 1H), 6.22 (td, J=2.0, 12.4 Hz, 1H), 6.14 (br d, J=14.4 Hz, 1H), 5.31 (s, 1H), 4.65-4.50 (m, 3H), 4.47 (br s, 2H), 4.26 (br s, 1H), 3.78 (br t, J=6.0 Hz, 1H), 3.56 (br t, J=6.0 Hz, 2H), 3.46-3.12 (m, 7H), 3.07-2.95 (m, 3H), 2.34 (br s, 2H), 2.22-2.12 (m, 1H), 1.92 (br d, J=13.2 Hz, 1H), 1.52 (s, 9H).

### Step 2—Tert-butyl (4R)-4-[4-[2-(dimethylcarbamoyl)-7-fluoro-6-[r-(3-pyrazol-1-ylpropanoyl)-3,6-dihydro-2H-pyridin-5-yl]-1H-indol-4-yl]-2,5-difluoro-phenyl]-3,3-difluoro-piperidine-1-carboxylate & tert-butyl (4S)-4-[4-[2-(dimethylcarbamoyl)-7-fluoro-6-[1-(3-pyrazol-1-ylpropanoyl)-3,6-dihydro-2H-pyridin-5-yl]-1H-indol-4-yl]-2,5-difluoro-phenyl]-3,3-difluoro-piperidine-1-carboxylate

4-[4-[2-(Dimethylcarbamoyl)-7-fluoro-6-[1-(3-pyrazol-1-ylpropanoyl)-3,6-dihydro-2H-pyridin-5-yl]-1H-indol-4-yl]-2,5-difluoro-phenyl]-3,3-difluoro-piperidine-1-carboxylate (1.4 g, 1.87 mmol) was separated by SFC (column: DAICEL CHIRALPAK IK (250 mm*30 mm, 10 um); mobile phase: [CO2-ACN/i-PrOH (0.1% NH₃H₂O)]; B %:40%, isocratic elution mode) to give the first eluting peak tert-butyl (4R)-4-[4-[2-(dimethylcarbamoyl)-7-fluoro-6-[1-(3-pyrazol-1-ylpropanoyl)-3,6-dihydro-2H-pyridin-5-yl]-1H-indol-4-yl]-2,5-difluoro-phenyl]-3,3-difluoro-piperidine-1-carboxylate (540 mg, 671 μmol) as a white solid and the second eluting peak tert-butyl (4S)-4-[4-[2-(dimethylcarbamoyl)-7-fluoro-6-[1-(3-pyrazol-1-ylpropanoyl)-3,6-dihydro-2H-pyridin-5-yl]-1H-indol-4-yl]-2,5-difluoro-phenyl]-3,3-difluoro-piperidine-1-carboxylate (460 mg, 602 μmol) as a white solid. LC-MS (ESI⁺) m, z 741.2 (M+H)⁺ for both isomers. The absolute stereochemistry of the enantiomers was assigned arbitrarily.

### 4-[4-[(4R)-3,3-difluoro-4-piperidyl]-2,5-difluoro-phenyl]-7-fluoro-N,N-dimethyl-6-[1-(3-pyrazol-1-ylpropanoyl)-3,6-dihydro-2H-pyridin-5-yl]-1H-indole-2-carboxamide (Intermediate MJ)

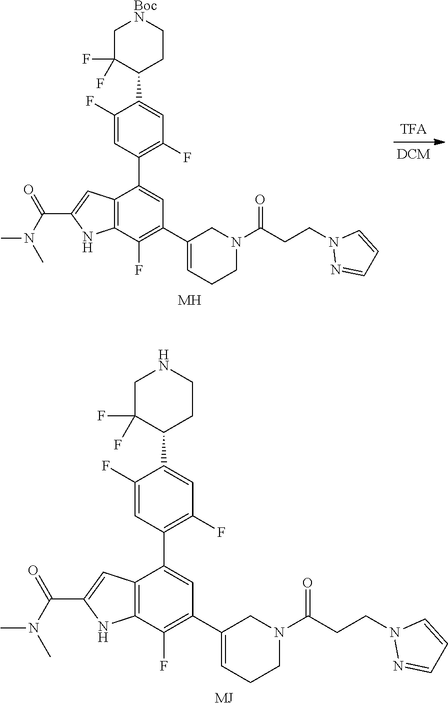

To a solution of tert-butyl (4R)-4-[4-[2-(dimethylcarbamoyl)-7-fluoro-6-[1-(3-pyrazol-1-ylpropanoyl)-3,6-dihydro-2H-pyridin-5-yl]-1H-indol-4-yl]-2,5-difluoro-phenyl]-3,3-difluoro-piperidine-1-carboxylate (540 mg, 728 μmol, Intermediate MH) in DCM (6 mL) was added TFA (3.07 g, 26.9 mmol, 2 mL). The mixture was then stirred at 25° C. for 2 hrs. On completion, the mixture was concentrated in vacuo. The crude residue was purified by reversed-phase HPLC (neutral condition) to give the title compound (430 mg, 88% yield) as a white solid. LC-MS (ESI⁺) m/z 641.3 (M+H)⁺.

### 4-[4-[(4S)-3,3-difluoro-4-piperidyl]-2,5-difluoro-phenyl]-7-fluoro-N,N-dimethyl-6-[1-(3-pyrazol-1-ylpropanoyl)-3,6-dihydro-2H-pyridin-5-yl]-1H-indole-2-carboxamide (Intermediate MK)

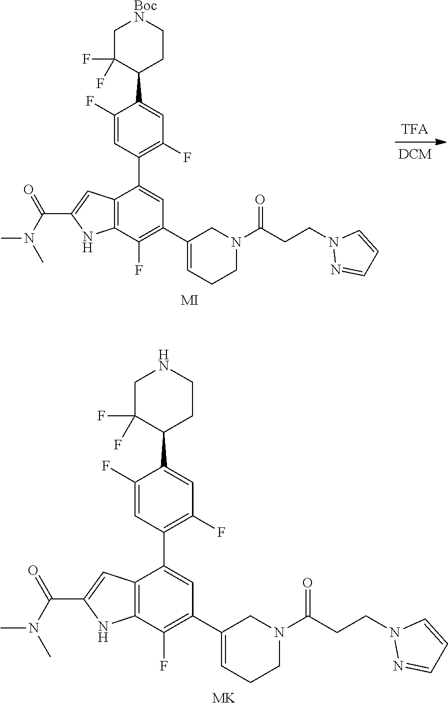

To a solution of tert-butyl (4S)-4-[4-[2-(dimethylcarbamoyl)-7-fluoro-6-[1-(3-pyrazol-1-ylpropanoyl)-3,6-dihydro-2H-pyridin-5-yl]-1H-indol-4-yl]-2,5-difluoro-phenyl]-3,3-difluoro-piperidine-1-carboxylate (460 mg, 621 μmol, Intermediate MI) in DCM (6 mL) was added TFA (3.07 g, 26.9 mmol, 2 mL). The mixture was stirred at 25° C. for 2 hrs. On completion, the mixture was concentrated in vacuo. The crude residue was purified by reversed-phase HPLC (neutral condition) to give the title compound (320 mg, 80% yield) as a white solid. LC-MS (ESI⁺) m/z 641.3 (M+H)⁺.

### 2′,6′-Bis(benzyloxy)-5-bromo-3-fluoro-4-methyl-2,3′-bipyridine (Intermediate ML)

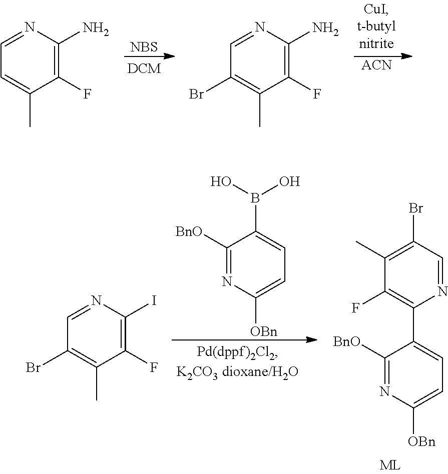

### Step 1—5-Bromo-3-fluoro-4-methyl-pyridin-2-amine

To a solution of 3-fluoro-4-methyl-pyridin-2-amine (4.5 g, 36 mmol. CAS #1003710-35-7) in DCM (80 mL) was added dropwise NBS (6.98 g, 39.2 mmol) in DCM (80 mL). After addition, the resulting mixture was stirred at 25° C. for 0.5 hr. On completion, the reaction mixture was quenched with 10% sodium sulfite solution (40 mL) and extracted with dichloromethane (3×100 mL). The combined organic layers were washed with brine (250 mL) and dried over anhydrous sodium sulfate, filtered and the filtrate was concentrated in vacuo to give the title compound (8.33 g) as a yellow solid. LC-MS (ESI⁺) m/z 204.9 (M+H)⁺.

### Step 2—5-Bromo-3-fluoro-2-iodo-4-methylpyridine

To a solution of 5-bromo-3-fluoro-4-methyl-pyridin-2-amine (7 g, 30 mmol) in ACN (100 mL) was added CuI (9.75 g. 51.2 mmol) and t-BuONO (5.28 g. 51.2 mmol). Then the mixture was stirred at 60° C. for 4 hrs. On completion, the reaction mixture was diluted with water (100 mL) and extracted with ethyl acetate (100×3 mL). The combined organic layers were washed with brine (200 mL) and dried over anhydrous sodium sulfate, filtered and the filtrate was concentrated in vacuo to give the crude residue. Then the residue was purified by column chromatography (SiO₂. Petroleum ether:Ethyl acetate=10/1) to give the title compound (2.9 g, 26% yield) as a brown solid. LC-MS (ESI⁺) m/z 317.8 (M+H)⁺.

### Step 3—2′,6′-Bis(benzyloxy)-5-bromo-3-fluoro-4-methyl-2,3′-bipyridine

To a solution of 5-bromo-3-fluoro-2-iodo-4-methyl-pyridine (2.5 g, 7.9 mmol) and (2,6-dibenzyloxy-3-pyridyl)boronic acid (2.65 g, 7.91 mmol, CAS #2096339-92-1) in dioxane (50 mL) and H₂O (10 mL) was added Pd(PPh₃)₄ (1.37 g. 1.19 mmol) and K₂CO₃ (3.28 g, 23.7 mmol). The mixture was then stirred at 100° C. for 12 hrs. On completion, the reaction mixture was diluted with water (50 mL) and extracted with ethyl acetate (50×3 mL). The combined organic layers were washed with brine (100 mL) and dried over anhydrous sodium sulfate, filtered and the filtrate was concentrated in vacuo to give the crude residue. Then the residue was purified by column chromatography (SiO₂, Petroleum ether:Ethyl acetate=4/1) to give the title compound (2.3 g, 59% yield) as a yellow solid. LC-MS (ESI⁺) m/z 480.4 (M+H)⁺.

### 3-(3-Fluoro-4-methyl-5-(4-(4-(4,4,5,5-tetramethyl-1,3,2-dioxaborolan-2-yl)phenyl)piperidin-1-yl)pyridin-2-yl)piperidine-2,6-dione (Intermediate MM)

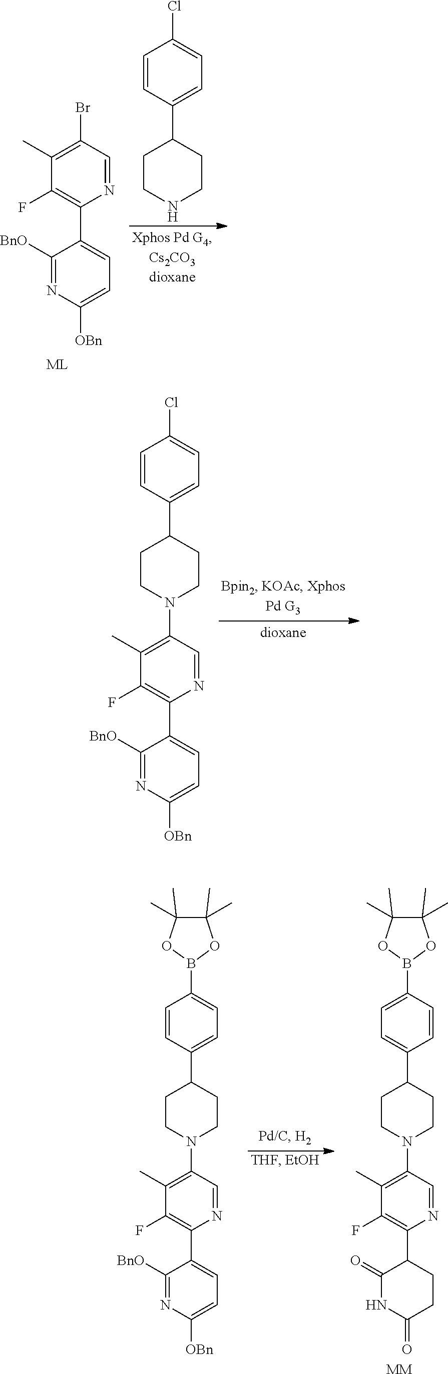

### Step 1—2′,6′-Bis(benzyloxy)-5-(4-(4-chlorophenyl)piperidin-1-yl)-3-fluoro-4-methyl-2,3′-bipyridine

To a solution of 5-bromo-2-(2,6-dibenzyloxy-3-pyridyl)-3-fluoro-4-methyl-pyridine (2.14 g, 4.46 mmol. Intermediate ML) and 4-(4-chlorophenyl)piperidine (961 mg, 4.91 mmol, CAS #26905-02-2) in dioxane (50 mL) was added Xphos Pd G4 (576 mg, 670 μmol) and Cs₂CO₃ (4.36 g, 13.4 mmol). The mixture was then stirred at 100° C. for 4 hrs. On completion, the reaction mixture was diluted with water (60 mL) and extracted with ethyl acetate (60×3 mL). The combined organic layers were washed with brine (120 mL) and dried over anhydrous sodium sulfate, filtered and the filtrate was concentrated in vacuo to give the crude residue. Then the residue was purified by column chromatography (SiO₂, Petroleum ether:Ethyl acetate=6/1) to give the title compound (800 mg, 24% yield) as a yellow solid. LC-MS (ESI⁺) m/z 594.3 (M+H)⁺.

### Step 2—2′,6′-Bis(benzyloxy)-3-fluoro-4-methyl-5-(4-(4-(4,4,5,5-tetramethyl-1,3,2-dioxaborolan-2-yl)phenyl)piperidin-1-yl)-2,3′-bipyridine

To a solution of 5-[4-(4-chlorophenyl)-1-piperidyl]-2-(2,6-dibenzyloxy-3-pyridyl)-3-fluoro-4-methyl-pyridine (700 mg, 1.18 mmol) and 4,4,5,5-tetramethyl-2-(4,4,5,5-tetramethyl-1,3,2-dioxaborolan-2-yl)-1,3,2-dioxaborolane (449 mg, 1.77 mmol) in dioxane (10 mL) was added KOAc (347 mg, 3.53 mmol) and XPhos Pd G3 (150 mg, 177 μmol). The mixture was then stirred at 80° C. for 3 hrs. On completion, the reaction mixture was concentrated in vacuo to remove the solvent. Then the residue was purified by column chromatography (SiO₂, Petroleum ether:Ethyl acetate=6/1) to give the title compound (500 mg, 61% yield) as a white solid. LC-MS (ESI⁺) m/z 686.5 (M+H)⁺.

### Step 3—3-(3-Fluoro-4-methyl-5-(4-(4-(4,4,5,5-tetramethyl-1,3,2-dioxaborolan-2-yl)phenyl)piperidin-1-yl)pyridin-2-yl)piperidine-2,6-dione

To a solution of 2-(2,6-dibenzyloxy-3-pyridyl)-3-fluoro-4-methyl-5-[4-[4-(4,4,5,5-tetramethyl-1,3,2-dioxaborolan-2-yl)phenyl]-1-piperidyl]pyridine (200 mg, 292 μmol) in THF (5 mL) and EtOH (5 mL) was added Pd/C (200 mg, 188 μmol). The mixture was then stirred at 40° C. for 12 hrs under H₂ atmosphere. On completion, the reaction mixture was concentrated in vacuo to give the title compound (200 mg) as a yellow solid. LC-MS (ESI⁺) m/z 508.2 (M+H)⁺.

### 3-(3-Fluoro-4-(piperidin-4-yl)phenyl)piperidine-2,6-dione (Intermediate MN)

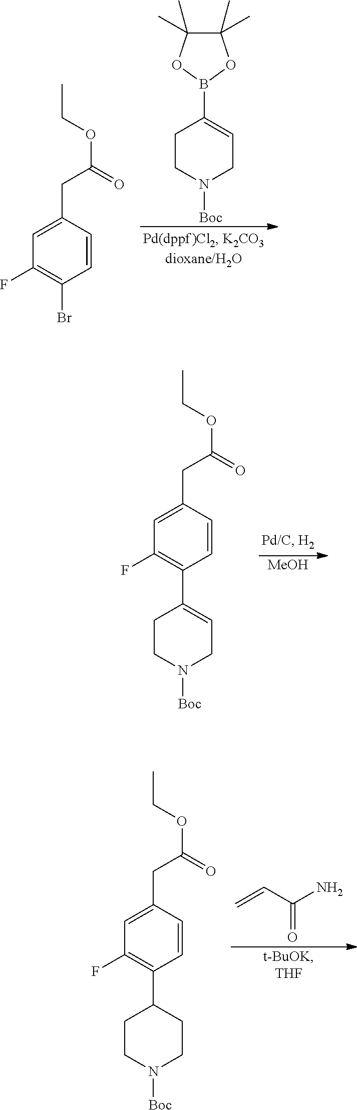

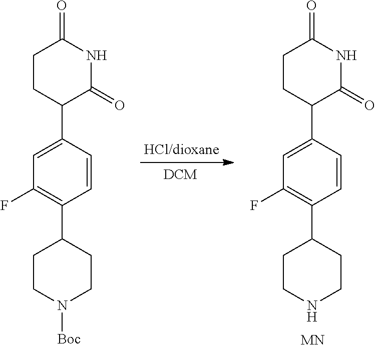 [SMILES: CC(C)(C)OC(=O)N1CCC(c2ccc(C3CCC(=O)NC3=O)cc2F)CC1 ; O=C1CCC(c2ccc(C3CCNCC3)c(F)c2)C(=O)N1]

### Step 1—Tert-butyl 4-(4-(2-ethoxy-2-oxoethyl)-2-fluorophenyl)-3,6-dihydropyridine-1 (2H)-carboxylate

To a solution of ethyl 2-(4-bromo-3-fluorophenyl)acetate (1.00 g, 3.83 mmol, CAS #1296223-82-9) in dioxane (10.0 mL) and H₂O (1.00 mL) was added tert-butyl 4-(4,4,5,5-tetramethyl-1,3,2-dioxaborolan-2-yl)-3,6-dihydropyridine-1 (2H)-carboxylate (1.42 g, 4.60 mmol, CAS #286961-14-6), K₂CO₃ (1.59 g, 11.5 mmol) and Pd(dppf)Cl₂ (280 mg, 383 μmol). Then the mixture was stirred at 80° C. for 2 h. On completion, the reaction mixture was concentrated under reduced pressure to give a residue. The residue was purified by flash silica gel chromatography (ISCO @; 20 g SepaFlash® Silica Flash Column, Eluent of 0˜20% Ethyl acetate/Petroleum ether gradient @60 mL/min) to afford the title compound (1.30 g, 86% yield) as a yellow gum, LC-MS (ESI⁻) m/z 386.0 (M+Na); ¹H NMR (400 MHz, CDCl₃) δ ppm 7.23-7.16 (m, 1H), 7.06-6.96 (m, 2H), 5.93 (s. 1H), 4.17 (q, J=7.2 Hz, 2H), 4.07 (d, J=2.8 Hz, 2H), 3.66-3.57 (m, 4H), 2.50 (s, 2H), 1.50 (s, 9H), 1.27 (t, J=7.2 Hz, 3H).

### Step 2—Tert-butyl 4-(4-(2-ethoxy-2-oxoethyl)-2-fluorophenyl)piperidine-1-carboxylate

To a solution of tert-butyl 4-(4-(2-ethoxy-2-oxoethyl)-2-fluorophenyl)-3,6-dihydropyridine-1 (2H)-carboxylate (1.20 g, 3.30 mmol) in MeOH (24.0 mL) was added Pd/C (120 mg, 10 wt %) under N₂ atmosphere. The suspension was degassed and purged with H₂ three times. The mixture was then stirred under H₂ (15 psi) at 25° C. for 2 h. On completion, the reaction mixture was filtered and concentrated under reduced pressure to give title compound (1.20 g, 100% yield) as a colorless oil. LC-MS (ESI⁺) m/z 387.9 (M+Na); ¹H NMR (400 MHz, CDCl₃) δ ppm 7.17-7.12 (m, 1H), 7.04-6.96 (m, 2H), 4.25 (d, J=13.2 Hz, 2H), 4.17 (q, J=7.2 Hz, 2H), 3.58 (s, 2H), 3.02-2.94 (m, 1H), 2.89-2.75 (m, 2H), 1.80 (d, J=12.8 Hz, 2H), 1.70-1.60 (m, 2H), 1.49 (s, 9H), 1.27 (t, J=7.2 Hz, 3H).

### Step 3—Tert-butyl 4-(4-(2,6-dioxopiperidin-3-yl)-2-fluorophenyl)piperidine-1-carboxylate

To a solution of tert-butyl 4-(4-(2-ethoxy-2-oxoethyl)-2-fluorophenyl)piperidine-1-carboxylate (1.15 g, 3.15 mmol) and acrylamide (201 mg, 2.83 mmol, CAS #9003-05-8) in THF (12.0 mL) was added t-BuOK (1 M, 3.46 mL) at 0° C., then the mixture was stirred at 50° C. for 2 h. On completion, the reaction mixture was poured into saturated NH₄Cl aqueous (50 mL), and extracted with EtOAc (30.0 mL×3). The combined organic layers were washed with brine (50.0 mL), and concentrated under reduced pressure to give a residue. The residue was purified by prep-HPLC (column: Waters Xbridge BEH C18 250*50 mm*10 um; mobile phase: [water (NH₄HCO₃)-ACN]; gradient: 37%-67% B over 10 min) and then lyophilized to dryness to afford the title compound (230 mg, 19% yield) as a white solid. LC-MS (ESI⁺) m/z 335.0 (M+H)⁺: ¹H NMR (400 MHz, CDCl₃) δ ppm 7.92 (s, 1H), 7.21 (t, J=7.6 Hz, 1H), 6.97 (d, J=8.0 Hz, 1H), 6.94-6.88 (m, 1H), 4.37-4.10 (m, 2H), 3.76 (dd, J=5.2, 10.0 Hz, 1H), 3.04-2.94 (m, 1H), 2.87-2.77 (m, 2H), 2.75 (t, J=5.2 Hz, 1H), 2.72-2.61 (m, 1H), 2.36-2.17 (m, 2H), 1.81 (d, J=11.6 Hz, 2H), 1.69-1.61 (m, 2H), 1.49 (s, 9H).

### Step 4—3-(3-Fluoro-4-(piperidin-4-yl)phenyl)piperidine-2,6-dione

To a solution of tert-butyl 4-(4-(2,6-dioxopiperidin-3-yl)-2-fluorophenyl)piperidine-1-carboxylate (130 mg, 333 μmol) in DCM (0.75 mL) was added HCl/dioxane (4 M, 750 μL) at 0° C., then the mixture was stirred at 20° C. for 1 h. On completion, the reaction mixture was concentrated under reduced pressure to give the title compound (110 mg, HCl) as a yellow solid. LC-MS (ESI⁺) m/z 290.8 (M+H)⁺.

### 3-(4-(1′-(4-Chloro-2,5-difluorophenyl)-[1,4′-bipiperidin]-4-yl)-3-fluorophenyl)piperidine-2,6-dione (Intermediate MO)

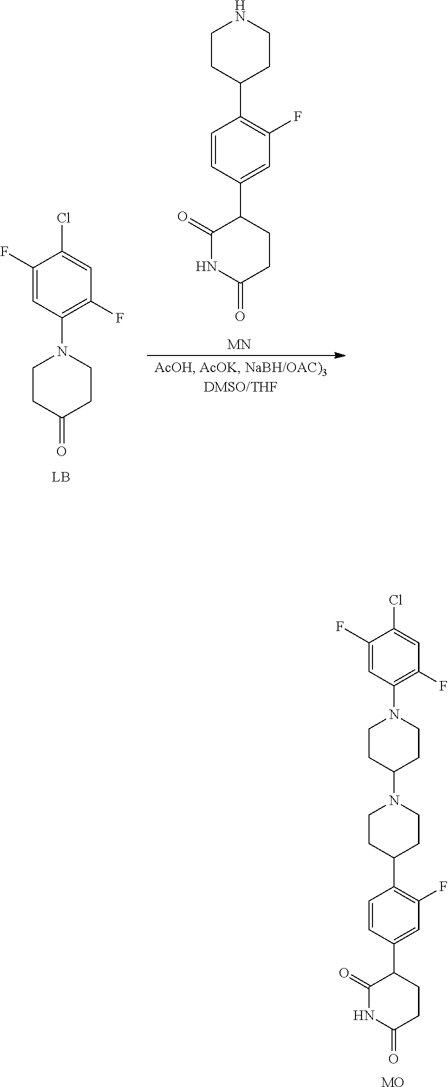

To a solution of 3-(3-fluoro-4-(piperidin-4-yl)phenyl)piperidine-2,6-dione (180 mg, 445 μmol, TFA, Intermediate MN) in DMSO (1 mL) and THF (1 mL) was added, AcOH (53.4 mg, 890 μmol, 50.9 μL), 1-(4-chloro-2,5-difluorophenyl)piperidin-4-one (109.3 mg, 445 μmol, Intermediate LB), and AcOK (87.3 mg, 890 μmol) at 25° C. Then the mixture was stirred at 40° C. for 1 hr. Next, NaBH₃CN (55.9 mg, 890 μmol) was added at 25° C., and the mixture was stirred at 25° C. for 5 hrs. On completion, the reaction mixture was quenched with water (10 mL) and extracted with ethyl acetate (3×10 mL). The combined organic layers were washed with brine (20 mL) and dried over anhydrous sodium sulfate, filtered and the filtrate was concentrated in vacuo to give the crude residue. The residue was purified by column chromatography (SiO₂, Petroleum ether/Ethyl acetate=5/1 to 0/1, THF/Ethyl acetate=1/10) to give the title compound (110 mg, 38% yield) as a yellow solid. LC-MS (ESI⁺) m/z 520.1 (M+H)⁺.

### 2,6-Bis(benzyloxy)-5′-fluoro-6′-(piperidin-4-yl)-3,3′-bipyridine (Intermediate MP)

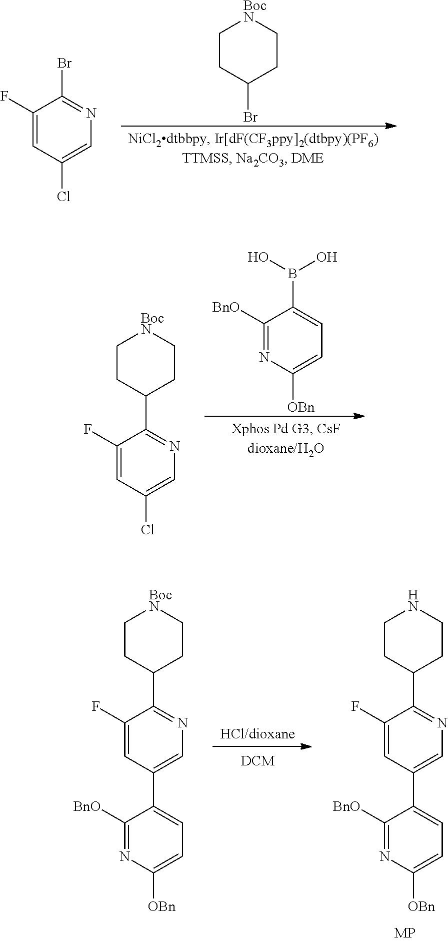 [SMILES: Fc1cc(Cl)cnc1Br ; CC(C)(C)OC(=O)N1CCC(Br)CC1 ; CC(C)(C)OC(=O)N1CCC(c2ncc(Cl)cc2F)CC1 ; OB(O)c1ccc(OCc2ccccc2)nc1OCc1ccccc1 ; CC(C)(C)OC(=O)N1CCC(c2ncc(-c3ccc(OCc4ccccc4)nc3OCc3ccccc3)cc2F)CC1 ; Fc1cc(-c2ccc(OCc3ccccc3)nc2OCc2ccccc2)cnc1C1CCNCC1]

### Step 1—Tert-butyl 4-(5-chloro-3-fluoropyridin-2-yl)piperidine-1-carboxylate

A mixture of 2-bromo-5-chloro-3-fluoro-pyridine (9 g, 40 mmol, CAS #514797-97-8), tert-butyl 4-bromopiperidine-1-carboxylate (11.3 g, 42.7 mmol), bis[3,5-difluoro-2-[5-(trifluoromethyl)-2-pyridyl]phenyl]iridium (1+); 4-tert-butyl-2-(4-tert-butyl-2-pyridyl)pyridine; hexafluorophosphate (479 mg, 427 μmol), 4-tert-butyl-2-(4-tert-butyl-2-pyridyl)pyridine; dichloronickel (255 mg, 641 μmol), bis(trimethylsilyl)silyl-trimethyl-silane (15.9 g. 64.1 mmol, 19.7 mL), and disodium carbonate (9 g, 90 mmol) in DME (45 mL) was degassed and purged with N₂ three times. Then the mixture was stirred at 25° C. for 12 hrs under N₂ atmosphere. On completion, the reaction mixture was diluted with water (200 mL) and extracted with EA (100 mL×2). The combined organic layers were washed with brine (100 mL×2), dried over Na₂SO₄, filtered and concentrated under reduced pressure to give a residue. The residue was purified by column chromatography (SiO₂, Petroleum ether/Ethyl acetate=20/1 to 10/1) to give a residue to give the title compound (3 g, 19% yield) as a white solid. LC-MS (ESI⁺) m/z 259.1 (M−55)⁺.

### Step 2—Tert-butyl 4-(2′,6′-bis(benzyloxy)-5-fluoro-[3,3′-bipyridin]-6-yl)piperidine-1-carboxylate

To a solution of tert-butyl 4-(5-chloro-3-fluoro-2-pyridyl)piperidine-1-carboxylate (2.7 g, 8.5 mmol) in dioxane (10 mL) and H₂O (10 mL) was added (2,6-dibenzyloxy-3-pyridyl)boronic acid (2.8 g, 8.5 mmol, CAS #2096339-92-1), XPhos Pd G3 (726 mg, 857 μmol) and CsF (3.9 g, 25.7 mmol). The mixture was stirred at 80° C. for 0.5 hr. On completion, the reaction mixture was diluted with water (100 mL) and extracted with EA (50 mL×2). The combined organic layers were washed with brine (50 mL×2), dried over Na₂SO₄, filtered and concentrated under reduced pressure to give a residue. The residue was purified by column chromatography (SiO₂, Petroleum ether/Ethyl acetate=20/1 to 5/1) to give a residue to give the title compound (2 g, 39% yield) as a white solid. LC-MS (ESI⁺) m/z 570.1 (M+H)⁺.

### Step 3—2,6-Bis(benzyloxy)-5′-fluoro-6′-(piperidin-4-yl)-3,3′-bipyridine

To a solution of tert-butyl 4-[5-(2,6-dibenzyloxy-3-pyridyl)-3-fluoro-2-pyridyl]piperidine-1-carboxylate (2 g, 4 mmol) in DCM (20 mL) was added HCl/dioxane (2 M, 1.76 mL). The mixture was then stirred at 25° C. for 0.5 hr. On completion, the reaction mixture was concentrated under reduced pressure to remove DCM. The residue was purified by prep-HPLC (HCl condition) to give the title compound (1.7 g, 96% yield, HCl) as a white solid. LC-MS (ESI⁺) m/z 470.1 (M+H)⁺.

### 3-(6-(1-(2,3-Difluoro-4-(4,4,5,5-tetramethyl-1,3,2-dioxaborolan-2-yl)phenyl)piperidin-4-yl)-5-fluoropyridin-3-yl)piperidine-2,6-dione (Intermediate MO)

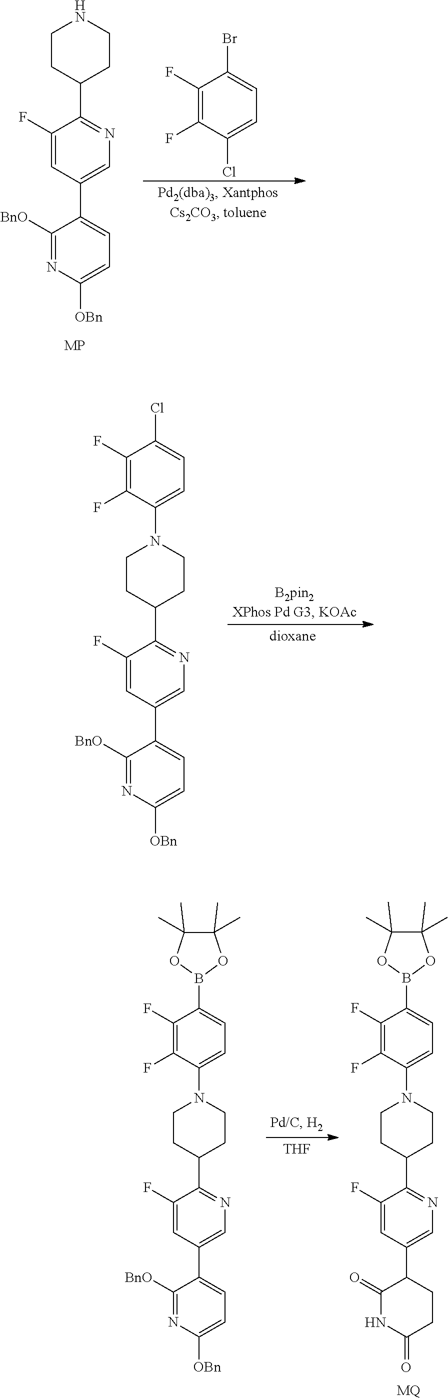

### Step 1—2,6-Bis(benzyloxy)-6′-(1-(4-chloro-2,3-difluorophenyl)piperidin-4-yl)-5′-fluoro-3,3′-bipyridine

To a solution of 2,6-dibenzyloxy-3-[5-fluoro-6-(4-piperidyl)-3-pyridyl]pyridine (1.6 g, 3.1 mmol, HCl, Intermediate MP) in toluene (18 mL) was added 1-bromo-4-chloro-2,3-difluoro-benzene (1 g, 4.4 mmol, CAS #1000574-47-9), Cs₂CO₃ (4.12 g, 12.6 mmol), Pd₂(dba)₃ (289 mg, 316 μmol) and XantPhos (365 mg, 632 μmol). The mixture was then stirred at 80° C. for 12 hrs. On completion, the reaction mixture was diluted with water (100 mL) and extracted with EA (50 mL×2). The combined organic layers were washed with brine (50 mL×2), dried over Na₂SO₄, filtered and concentrated under reduced pressure to give a residue. The residue was purified by column chromatography (SiO₂, Petroleum ether/Ethyl acetate=40/1 to 20/1) to give the title compound (1.3 g, 67% yield) as a white solid. LC-MS (ESI⁺) m/z 616.5 (M+H)⁺.

### Step 2—2,6-Bis(benzyloxy)-6′-(1-(2,3-difluoro-4-(4,4,5,5-tetramethyl-1,3,2-dioxaborolan-2-yl)phenyl)piperidin-4-yl)-5′-fluoro-3,3′-bipyridine

To a solution of 2,6-dibenzyloxy-3-[6-[1-(4-chloro-2,3-difluoro-phenyl)-4-piperidyl]-5-fluoro-3-pyridyl]pyridine (1.15 g, 1.8 mmol) in dioxane (10 mL) was added 4,4,5,5-tetramethyl-2-(4,4,5,5-tetramethyl-1,3,2-dioxaborolan-2-yl)-1,3,2-dioxaborolane (3.8 g, 15 mmol), XPhos Pd G₃ (158 mg, 186.6 μmol) and AcOK (549 mg, 5.6 mmol). Then the mixture was stirred at 90° C. for 0.5 hr. On completion, the reaction mixture was concentrated under reduced pressure to remove dioxane. The residue was purified by column chromatography (SiO₂, Petroleum ether/Ethyl acetate=40/1 to 20/1) to give the title compound (550 mg, 37% yield) as a white solid. LC-MS (ESI⁺) m/z 708.3 (M+H)⁺.

### Step 3—3-(6-(1-(2,3-Difluoro-4-(4,4,5,5-tetramethyl-1,3,2-dioxaborolan-2-yl)phenyl)piperidin-4-yl)-5-fluoropyridin-3-yl)piperidine-2,6-dione

To a solution of 2,6-dibenzyloxy-3-[6-[1-[2,3-difluoro-4-(4,4,5,5-tetramethyl-1,3,2-dioxaborolan-2-yl)phenyl]-4-piperidyl]-5-fluoro-3-pyridyl]pyridine (500 mg, 707 μmol) in THF (10 mL) was added Pd/C (751 mg, 706 μmol, 70.6 μL, 10 wt %). The mixture was then stirred at 30° C. for 12 hrs under H₂ (40 psi). On completion, the reaction mixture was filtered and concentrated under reduced pressure give the title product (330 mg, 37% yield) as a white solid. LC-MS (ESI⁺) m/z 530.3 (M+H)⁺.

### 7-(4-Chloro-2-fluorophenyl)-2,7-diazaspiro[3.5]nonane (Intermediate MR)

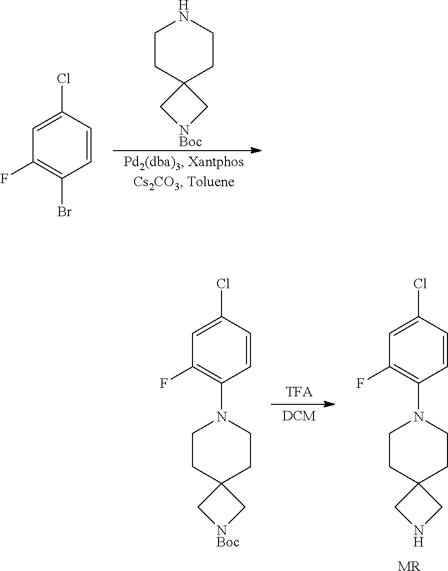

### Step 1—Tert-butyl 7-(4-chloro-2-fluorophenyl)-2,7-diazaspiro[3.5]nonane-2-carboxylate

A mixture of 1-bromo-4-chloro-2-fluoro-benzene (2.78 g, 13.3 mmol, CAS #1996-29-8), tert-butyl 2,7-diazaspiro[3.5]nonane-2-carboxylate (3 g, 13.3 mmol, CAS #236406-55-6), Cs₂CO₃ (10.8 g, 33.1 mmol), Xantphos (1.15 g, 1.99 mmol) and Pd₂(dba)₃ (850 mg, 928 μmol) in toluene (30 mL) was degassed and purged with N₂ three times. Then the mixture was stirred at 80° C. for 12 hr under N₂ atmosphere. On completion, the reaction mixture was concentrated under reduced pressure. The mixture was purified by column chromatography (SiO₂, Petroleum ether/Ethyl acetate=10/1 to 4/1) to give the title compound (3.81 g, 81% yield) as a white solid. LC-MS (ESI⁺) m/z 355.1 (M+H)⁺.

### Step 2—7-(4-Chloro-2-fluorophenyl)-2,7-diazaspiro[3.5]nonane

To a solution of tert-butyl 7-(4-chloro-2-fluoro-phenyl)-2,7-diazaspiro[3.5]nonane-2-carboxylate (2 g, 6 mmol) in DCM (20 mL) was added TFA (7.68 g, 67.31 mmol, 5 mL). The mixture was then stirred at 20° C. for 4 h. On completion, the reaction mixture was filtered and concentrated under reduced pressure to give a residue. The crude product was purified by reversed-phase HPLC (0.1% FA condition) to give the title compound (755 mg, 52% yield) as a yellow solid. LC-MS (ESI⁺) m/z 255.1 (M+H)⁺.

### Methyl 4,5-difluoro-2-methoxybenzoate (Intermediate MS)

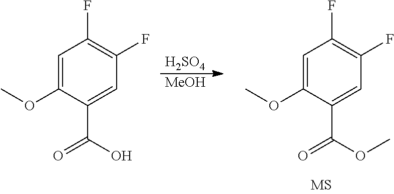

To a solution of 4,5-difluoro-2-methoxy-benzoic acid (400 mg, 2.13 mmol, CAS #425702-18-7) in MeOH (6 mL) was added H₂SO₄ (368 mg, 3.75 mmol, 0.2 mL). The mixture was then stirred at 80° C. for 16 h. On completion, the reaction mixture was filtered and concentrated under reduced pressure to give a residue. The crude product was purified by column chromatography (SiO₂, Petroleum ether/Ethyl acetate=10/1) to give the title compound (390 mg, 91% yield) as a white solid. ¹H NMR (400 MHz. DMSO-d₆) δ=3.79 (s, 3H) 3.83 (s, 3H) 7.34 (dd, J=12.8, 6.4 Hz, 1H) 7.73 (dd. J=10.4, 9.6 Hz. 1H).

### 4-(7-(4-Chloro-2-fluorophenyl)-2,7-diazaspiro[3.5]nonan-2-yl)-5-fluoro-2-methoxybenzoic acid (Intermediate MT)

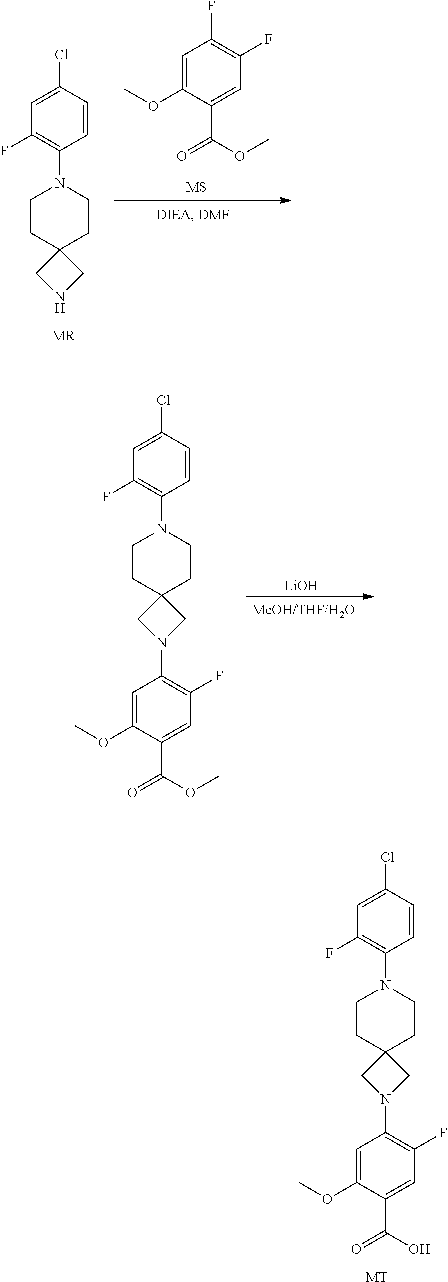

### Step 1—Methyl 4-[7-(4-chloro-2-fluoro-phenyl)-2,7-diazaspiro[3.5]nonan-2-yl]-5-fluoro-2-methoxy-benzoate

A mixture of methyl 4,5-difluoro-2-methoxy-benzoate (300 mg, 1.48 mmol, Intermediate MS), 7-(4-chloro-2-fluoro-phenyl)-2,7-diazaspiro[3.5]nonane (378 mg, 1.48 mmol, Intermediate MR), and DIEA (1.92 g. 14.8 mmol, 2.58 mL) in DMF (6 mL) was degassed and purged with N₂ three times. Then the mixture was stirred at 120° C. for 16 hr under N₂ atmosphere. On completion, the reaction mixture was filtered and concentrated under reduced pressure to give a residue. The crude product was purified by column chromatography (SiO₂, Petroleum ether/Ethyl acetate=5/1 to 2/1) to give the title compound (220 mg, 34% yield) as a yellow solid. LC-MS (ESI⁺) m/z 437.1 (M+H)⁺.

### Step 2—4-(7-(4-Chloro-2-fluorophenyl)-2,7-diazaspiro[3.5]nonan-2-yl)-5-fluoro-2-methoxybenzoic acid

To a solution of methyl 4-[7-(4-chloro-2-fluoro-phenyl)-2,7-diazaspiro[3.5]nonan-2-yl]-5-fluoro-2-methoxy-benzoate (220 mg, 503 μmol) in MeOH (2 mL), THF (4 mL) and H₂O (2 mL) was added LiOH·H₂O (106 mg. 2.52 mmol). The mixture was stirred at 45° C. for 12 h. On completion, the reaction mixture was added TFA until the pH was 4, then the reaction mixture was extracted with dichloromethane (5×3 mL). The combined organic layers were washed with brine (50 mL), dried over anhydrous sodium sulfate, filtered and the filtrate was concentrated in vacuo to give the title compound (210 mg) as a yellow solid. LC-MS (ESI⁺) m/z 423.1 (M+H)⁺.

### 4-(7-(4-Chloro-2-fluorophenyl)-2,7-diazaspiro[3.5]nonan-2-yl)-N-(2,6-dioxopiperidin-3-yl)-5-fluoro-2-methoxybenzamide (Intermediate MU)

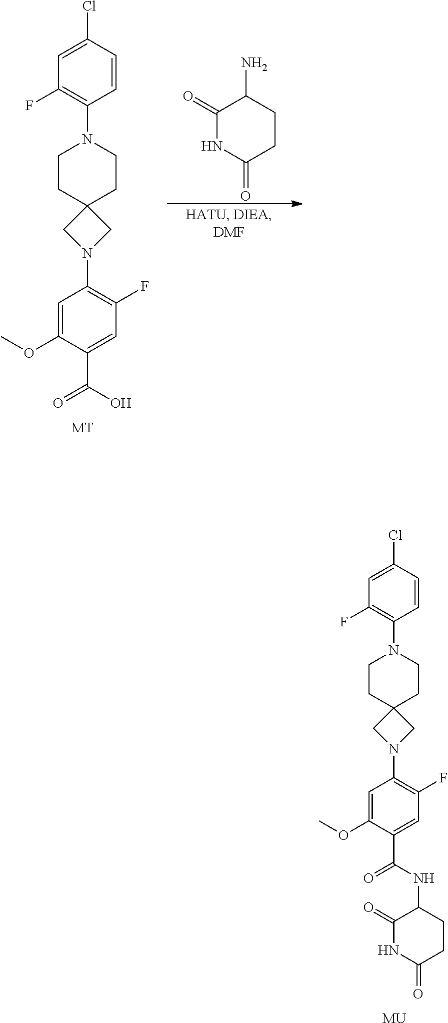

To a solution of 4-[7-(4-chloro-2-fluoro-phenyl)-2,7-diazaspiro[3.5]nonan-2-yl]-5-fluoro-2-methoxy-benzoic acid (190 mg, 449 μmol, Intermediate MT) in DMF (2 mL) was added HATU (205 mg, 539 μmol), DIEA (290 mg, 2.25 mmol, 391 μL) and 3-aminopiperidine-2,6-dione (57.6 mg, 449 μmol, CAS #2353-44-8). The mixture was then stirred at 40° C. for 1 h. On completion, the reaction mixture was concentrated in vacuo to give the crude residue. The residue was purified by column chromatography (SiO₂, Petroleum ether/Ethyl acetate=0/1 to THF) to give the title compound (184 mg, 77% yield) as a white solid. LC-MS (ESI⁺) m/z 533.2 (M+H)⁺.

### 7-(4-Chloro-2,3-difluorophenyl)-2,7-diazaspiro[3.5]nonane (Intermediate MV)

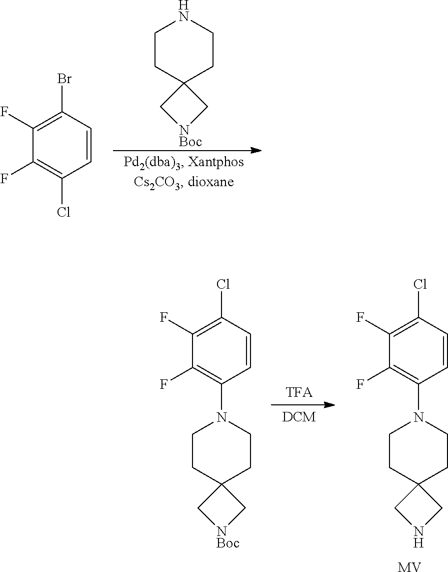

### Step 1—Tert-butyl 7-(4-chloro-2,3-difluorophenyl)-2,7-diazaspiro[3.5]nonane-2-carboxylate

A mixture of 1-bromo-4-chloro-2,3-difluorobenzene (2.7 g, 12 mmol, CAS #1000574-47-9), tert-butyl 2,7-diazaspiro[3.5]nonane-2-carboxylate (2.69 g, 11.9 mmol, CAS #236406-55-6), Xantphos (687 mg, 1.2 mmol), Pd₂(dba)₃ (1.09 g, 1.2 mmol) and Cs₂CO₃ (11.6 g, 35.6 mmol) in dioxane (40 mL) was degassed and purged with N₂ three times. Then the mixture was stirred at 80° C. for 3 hrs under N₂ atmosphere. On completion, the mixture was diluted with water (50 mL), extracted with ethyl acetate (50 mL×2), dried over sodium sulfate, filtered and concentrated under reduced pressure. The residue was purified by silica gel column chromatography (petroleum ether/ethyl acetate=1/0˜9/1) to give the title compound (4 g, 73% yield) as a yellow solid. LC-MS (ESI⁺) m/z 373.0 (M+H)⁺.

### Step 2—7-(4-Chloro-2,3-difluorophenyl)-2,7-diazaspiro[3.5]nonane

To a solution of tert-butyl 7-(4-chloro-2,3-difluorophenyl)-2,7-diazaspiro[3.5]nonane-2-carboxylate (4 g, 7 mmol) in DCM (20 mL) was added TFA (5 mL). The mixture was then stirred at 25° C. for 4 hrs. On completion, the mixture was filtered and concentrated under reduced pressure to give a residue. The residue was purified by prep-HPLC (neutral condition) to give the title compound (1.5 g, 82% yield) as a white solid. LC-MS (ESI⁺) m/z 272.8 (M+H)⁻.

### 4-(7-(4-Chloro-2,3-difluorophenyl)-2,7-diazaspiro[3.5]nonan-2-yl)-5-fluoro-2-methoxybenzoic acid (Intermediate MW)

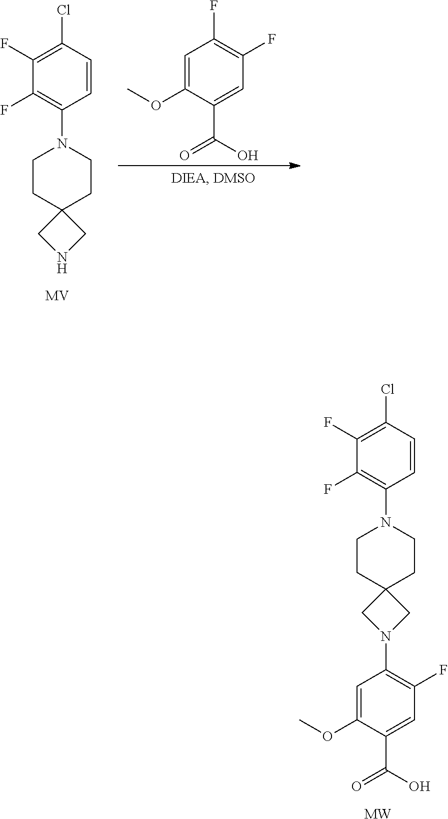

To a solution of 4,5-difluoro-2-methoxybenzoic acid (369 mg, 1.96 mmol, CAS #425702-18-7) and 7-(4-chloro-2,3-difluorophenyl)-2,7-diazaspiro[3.5]nonane (535 mg, 1.96 mmol, Intermediate MV) in DMSO (20 mL) was added DIEA (2.54 g, 19.63 mmol). Then the mixture was stirred at 100° C. for 2 hrs. On completion, the reaction mixture was concentrated under reduced pressure to give a residue. The residue was purified by prep-HPLC (FA condition) to give the title compound (206 mg, 22% yield) as a white solid. LC-MS (ESI⁺) m/z 441.0 (M+H)⁺.

### 4-(7-(4-Chloro-2,3-difluorophenyl)-2,7-diazaspiro[3.5]nonan-2-yl)-N-(2,6-dioxopiperidin-3-yl)-5-fluoro-2-methoxybenzamide (Intermediate MX)

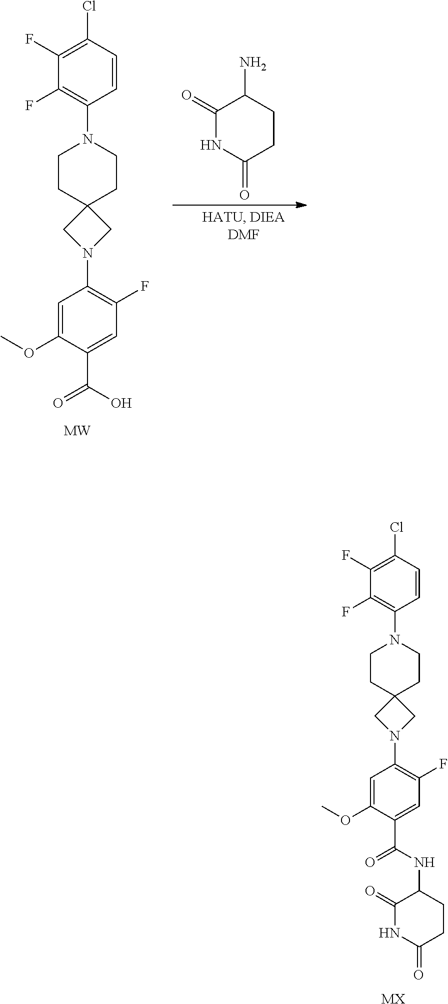

To a solution of 4-[7-(4-chloro-2,3-difluoro-phenyl)-2,7-diazaspiro[3.5]nonan-2-yl]-5-fluoro-2-methoxy-benzoic acid (196 mg, 444 μmol, Intermediate MW), 3-aminopiperidine-2,6-dione (57.0 mg, 444 μmol, CAS #2353-44-8) in DMF (4 mL) was added HATU (253 mg, 667 μmol) and DIEA (172 mg, 1.33 mmol). Then the mixture was stirred at 25° C. for 1 hr. On completion, the mixture was diluted with H₂O (20 mL), extracted with DCM (50 mL×2), dried over sodium sulfate, filtered and concentrated under reduced pressure. The residue was purified by prep-HPLC (FA condition) to give the title compound (80 mg, 22% yield) as a white solid. LC-MS (ESI⁺) m/z 551.0 (M+H)⁺.

### 3-(4-(4-(4-Chloro-2,5-difluorophenyl)piperazin-1-yl)-3-fluorophenyl)piperidine-2,6-dione (Intermediate MY)

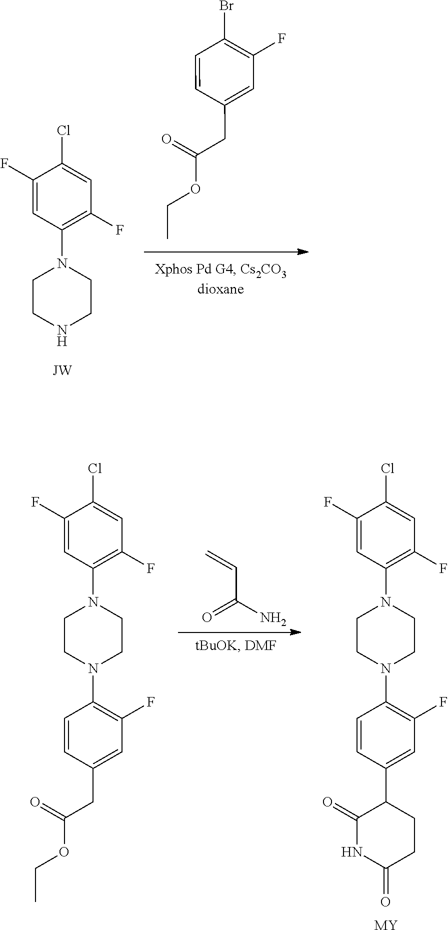

### Step 1—Ethyl 2-(4-(4-(4-chloro-2,5-difluorophenyl)piperazin-1-yl)-3-fluorophenyl)acetate

To a solution of ethyl 2-(4-bromo-3-fluorophenyl)acetate (1.36 g, 5.20 mmol, CAS #1296223-82-9) and 1-(4-chloro-2,5-difluorophenyl)piperazine (1.4 g, 5.2 mmol, HCl, Intermediate JW) in dioxane (20 mL) was added Xphos Pd G4 (447 mg, 520 μmol), Cs₂CO₃ (8.47 g, 26.0 mmol) and 4 Å molecular sieves (5.20 mmol) at 25° C. under nitrogen atmosphere. Then the mixture was stirred at 100° C. for 5 hrs under nitrogen atmosphere. On completion, the reaction mixture was concentrated in vacuo to give the crude residue. The residue was purified by column chromatography (SiO₂, Petroleum ether/Ethyl acetate=8/l to 4/1) to give the title compound (1 g, 22% yield) as a yellow solid. LC-MS (ESI⁺) m/z 413.0 (M+H)⁺.

### Step 2—3-(4-(4-(4-Chloro-2,5-difluorophenyl)piperazin-1-yl)-3-fluorophenyl)piperidine-2,6-dione

To a solution of acrylamide (258 mg, 3.63 mmol, 250 mL, CAS #9003-05-8) and ethyl 2-(4-(4-(4-chloro-2,5-difluorophenyl)piperazin-1-yl)-3-fluorophenyl)acetate (1 g, 2 mmol) in DMF (10 mL) was added t-BuOK (1 M, 2.66 mL) at 0° C. under nitrogen atmosphere. Then the mixture was stirred at 25° C. for 15 mins under nitrogen atmosphere. On completion, the reaction mixture was quenched by 1N HCl (50 mL), then the reaction mixture was filtered and the filter cake was dried in vacuo to give the title compound (1 g, HCl) as a yellow solid. LC-MS (ESI⁺) m/z 438.1 (M+H)⁺.

### 3-(4-(1-(4-Bromo-2,5-difluorophenyl)piperidin-4-yl)-3-fluorophenyl)piperidine-2,6-dione (Intermediate MZ)

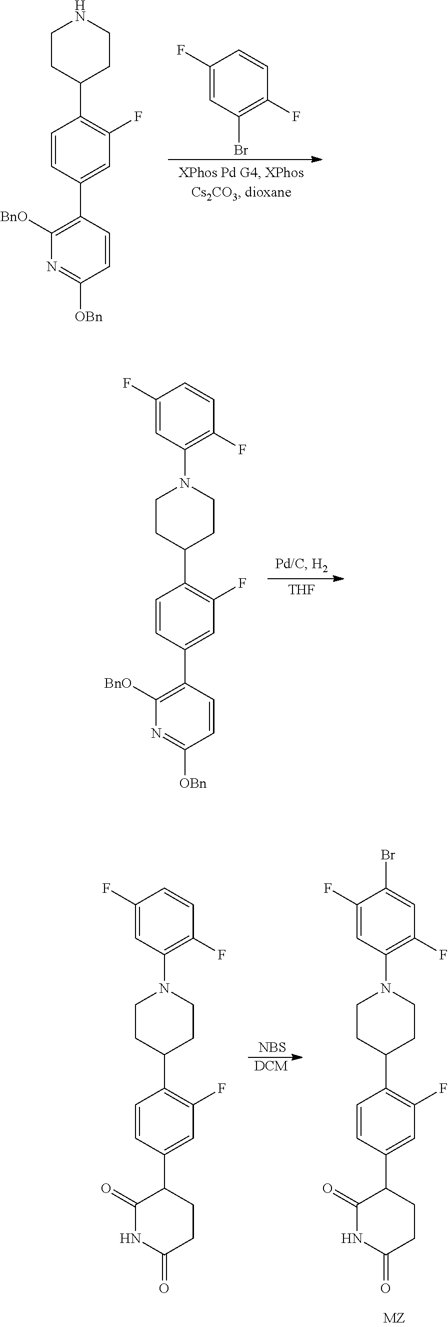

### Step 1—2,6-Bis(benzyloxy)-3-(4-(1-(2,5-difluorophenyl)piperidin-4-yl)-3-fluorophenyl)pyridine

To a solution of 2,6-bis(benzyloxy)-3-(3-fluoro-4-(piperidin-4-yl)phenyl)pyridine (1 g, 2 mmol, synthesized via Step 1 of Intermediate KT), 2-bromo-1,4-difluoro-benzene (453 mg, 2.35 mmol, CAS #399-94-0) in dioxane (10 mL) was added XPhos (102 mg, 213 μmol), Xphos Pd G4 (184 mg, 213 μmol), and Cs₂CO₃ (2.09 g, 6.40 mmol). The mixture was then stirred at 80° C. for 12 hrs. On completion, the reaction mixture was quenched with water (30 mL) and extracted with dichloromethane (3×30 mL). The combined organic layers were washed with aqueous NaCl (30 mL) and dried over anhydrous sodium sulfate, filtered and the filtrate was concentrated in vacuo to give the crude residue. The residue was purified by column chromatography (SiO₂, Petroleum ether:Ethyl acetate=19/1) to give the title compound (860 mg, 69% yield) as yellow oil. LC-MS (ESI⁺) m/z 581.2 (M+H)⁺.

### Step 2—3-(4-(1-(2,5-Difluorophenyl)piperidin-4-yl)-3-fluorophenyl)piperidine-2,6-dione

A mixture of 2,6-bis(benzyloxy)-3-(4-(1-(2,5-difluorophenyl)piperidin-4-yl)-3-fluorophenyl)pyridine (860 mg, 1.48 mmol), Pd/C (6.98 g, 6.56 mmol, 10 wt %) in THF (2 mL) was degassed and purged with H₂ three times. Then the mixture was stirred at 25° C. for 48 hrs under H₂ atmosphere. On completion, the reaction was filtered very carefully, the filtrated was concentrated in vacuo to give the title compound (500 mg) as a white solid. LC-MS (ESI⁺) m/z 403.1 (M+H)⁺.

### Step 3—3-(4-(1-(4-Bromo-2,5-difluorophenyl)piperidin-4-yl)-3-fluorophenyl)piperidine-2,6-dione

To a solution of 3-(4-(1-(2,5-difluorophenyl)piperidin-4-yl)-3-fluorophenyl)piperidine-2,6-dione (200 mg, 413 μmol) in DCM (2 mL) was added NBS (73.4 mg, 413 μmol) at 0° C. The mixture was stirred at 25° C. for 3 hrs. On completion, the reaction mixture was quenched with water (30 mL) and extracted with dichloromethane (3×30 mL). The combined organic layers were washed with aqueous NaCl (30 mL) and dried over anhydrous sodium sulfate, filtered and the filtrate was concentrated in vacuo to give the crude residue. The crude residue was purified by reversed-phase HPLC (0.1% FA condition) to give the title compound (70 mg, 35% yield) as a white solid. LC-MS (ESI⁺) m/z 482.0 (M+H)˜.

### 2,6-Bis(benzyloxy)-3-(4-bromo-3-chlorophenyl)pyridine (Intermediate NA)

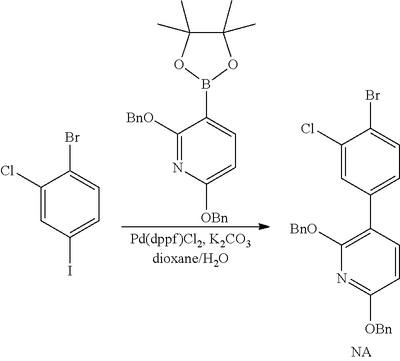 [SMILES: Clc1cc(I)ccc1Br ; CC1(C)OB(c2ccc(OCc3ccccc3)nc2OCc2ccccc2)OC1(C)C ; Clc1cc(-c2ccc(OCc3ccccc3)nc2OCc2ccccc2)ccc1Br]

To a solution of 1-bromo-2-chloro-4-iodo-benzene (2 g, 6 mmol) in dioxane (10 mL) and H₂O (10 mL) was added 2,6-dibenzyloxy-3-(4,4,5,5-tetramethyl-1,3,2-dioxaborolan-2-yl)pyridine (2.63 g, 6.3 mmol, synthesized via Step 1 of Intermediate AT), Pd(dppf)Cl₂CH₂Cl₂ (514.6 mg, 630.2 μmol) and K₂CO₃ (2.61 g, 18.9 mmol). The mixture was then stirred at 60° C. for 0.5 hr. On completion, the reaction mixture was concentrated under reduced pressure to remove dioxane. The residue was purified by column chromatography (SiO₂, Petroleum ether/Ethyl acetate=40/1 to 10/1) to give a residue to give the title compound (2.4 g, 75% yield) as a white solid. LC-MS (ESI⁺) m/z 482.0 (M+H)⁺.

### 4-(1-(2-Chloro-4-(2,6-dioxopiperidin-3-yl)phenyl)piperidin-4-yl)phenyl trifluoromethanesulfonate (Intermediate NB)

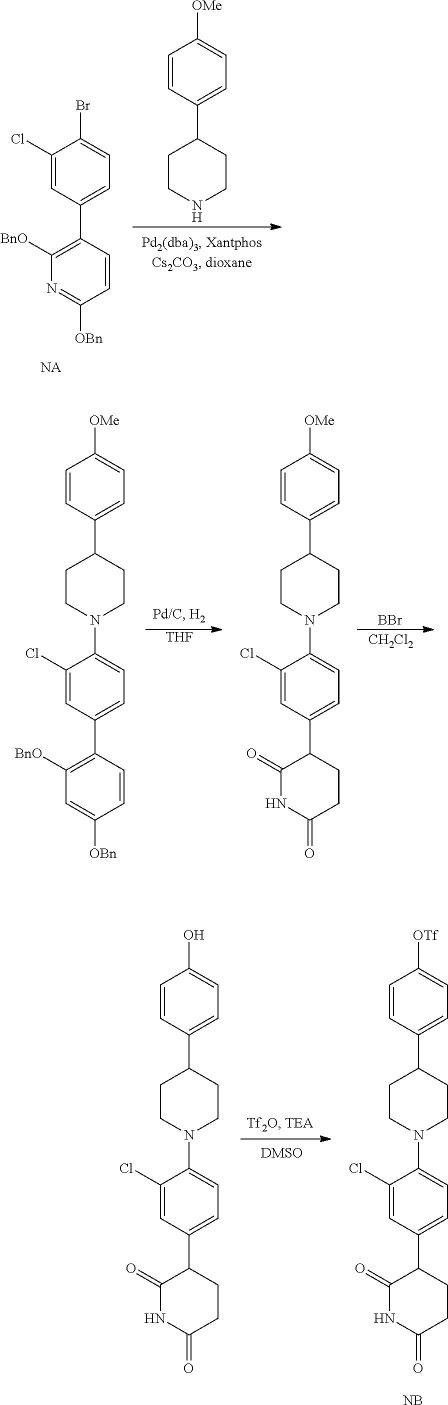

### Step 1—2,6-Bis(benzyloxy)-3-(3-chloro-4-(4-(4-methoxyphenyl)piperidin-1-yl)phenyl)pyridine

To a solution of 2,6-dibenzyloxy-3-(4-bromo-3-chloro-phenyl)pyridine (1.4 g, 2.9 mmol, Intermediate NA) in toluene (14 mL) was added 4-(4-methoxyphenyl)piperidine (928.3 mg, 4 mmol, HCl, CAS #6748-48-7), Xantphos (336.9 mg, 582.3 μmol), Cs₂CO₃ (3.80 g, 11.6 mmol) and Pd₂(dba)₃ (266.6 mg, 291.1 μmol). The mixture was then stirred at 100° C. for 12 hrs. On completion, the reaction mixture was diluted with water (100 mL) and extracted with EA (50 mL×2). The combined organic layers were washed with brine (50 mL×2), dried over Na₂SO₄, filtered and concentrated under reduced pressure to give a residue. The residue was purified by column chromatography (SiO₂, Petroleum ether/Ethyl acetate=20/1 to 5/1) to give the title compound (770 mg, 31% yield) as a white solid. LC-MS (ESI⁺) m/z 591.2 (M+H)

### Step 2—3-(3-Chloro-4-(4-(4-methoxyphenyl)piperidin-1-yl)phenyl)piperidine-2,6-dione

To a solution of 2,6-dibenzyloxy-3-[3-chloro-4-[4-(4-methoxyphenyl)-1-piperidyl]phenyl]pyridine (770 mg, 1.3 mmol) in THF (10 mL) was added Pd/C (770 mg, 723 μmol, 10 wt %). The mixture was stirred at 40° C. for 12 hours under 45 psi of H₂. On completion, the reaction mixture was concentrated under reduced pressure to give the title compound (500 mg) as a white solid. LC-MS (ESI⁺) m/z 413.1 (M+H)⁺.

### Step 3—3-(3-Chloro-4-(4-(4-hydroxyphenyl)piperidin-1-yl)phenyl)piperidine-2,6-dione

To a solution of 3-[3-chloro-4-[4-(4-methoxyphenyl)-1-piperidyl]phenyl]piperidine-2,6-dione (450 mg, 1 mmol) in CH₂Cl₂ (5 mL) was added BBr₃ (2 M, 5.45 mL). The mixture was stirred at 10° C. for 1 hr, then the mixture was stirred at 0° C. for 2 hrs. On completion, the reaction mixture was extracted with EA (50 mL×2). The combined organic layers were washed with solvent (50 mL×2), dried over Na₂SO₄, filtered and concentrated under reduced pressure to give the title compound (450 mg) as a white solid. LC-MS (ESI+) m/z 399.0 (M+H)⁺.

### Step 4—4-(1-(2-Chloro-4-(2,6-dioxopiperidin-3-yl)phenyl)piperidin-4-Yl)phenyl trifluoromethanesulfonate

To a solution of 3-[3-chloro-4-[4-(4-hydroxyphenyl)-1-piperidyl]phenyl]piperidine-2,6-dione (400 mg, 1.00 mmol) in DMSO (4 mL) was added 1,1,1-trifluoro-N-phenyl-N-(trifluoromethylsulfonyl)methanesulfonamide (716.50 mg, 2.01 mmol) and Et₃N (304.4 mg, 3 mmol, 418.7 μL). The mixture was then stirred at 60° C. for 1 hr. On completion, the reaction mixture was diluted with water (100 mL) and extracted with EA (50 mL×2). The combined organic layers were washed with brine (50 mL×2), dried over Na₂SO₄, filtered and concentrated under reduced pressure to give a residue. The residue was purified by column chromatography (SiO₂, Petroleum ether/Ethyl acetate=50/1 to 10/1) to give the title compound (235 mg, 32% yield) as a white solid. LC-MS (ESI⁺) m/z 531.1 (M+H)⁺.

### 3-(4-(4-(4-Chloro-2-fluorophenyl)piperidin-1-yl)-3-fluorophenyl)piperidine-2,6-dione (Intermediate NC)

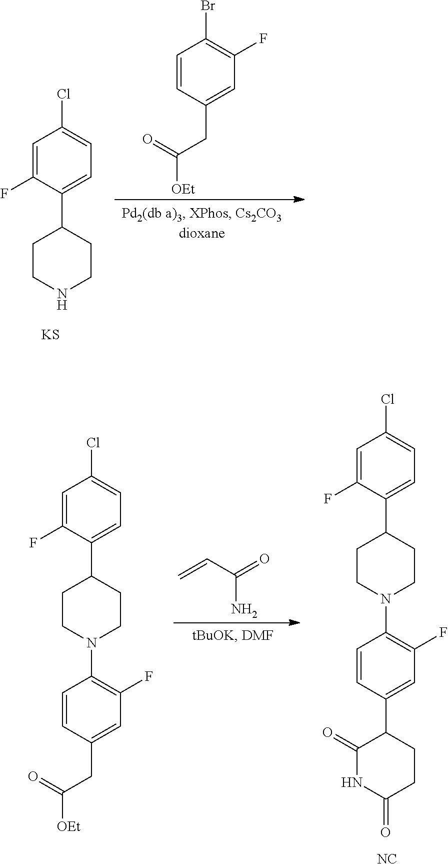

### Step 1—Ethyl 2-(4-(4-(4-chloro-2-fluorophenyl)piperidin-1-yl)-3-fluorophenyl)acetate

A mixture of 4-(4-chloro-2-fluoro-phenyl)piperidine (500 mg, 2.34 mmol, Intermediate KS), ethyl 2-(4-bromo-3-fluoro-phenyl)acetate (916 mg, 3.51 mmol, CAS #1296223-82-9), Cs₂CO₃ (2.29 g, 7.02 mmol), XPhos (112 mg, 234 μmol) and Pd₂(dba)₃ (107 mg, 117 μmol) in dioxane (10 mL) was degassed and purged with N₂ three times. Then the mixture was stirred at 100° C. for 1 hr under N₂ atmosphere. On completion, the reaction mixture was filtered and concentrated under reduced pressure to give a residue. The crude residue was purified by column chromatography (SiO₂, Petroleum ether/Ethyl acetate=20/1 to 5/1) to give the title compound (740 mg, 51% yield) as a yellow solid. LC-MS (ESI⁺) m/z 394.1 (M+H)⁺.

### Step 2—3-(4-(4-(4-Chloro-2-fluorophenyl)piperidin-1-yl)-3-fluorophenyl)piperidine-2,6-dione

To a solution of ethyl 2-[4-[4-(4-chloro-2-fluoro-phenyl)-1-piperidyl]-3-fluoro-phenyl]acetate (640 mg, 1.62 mmol) and prop-2-enamide (139 mg, 1.95 mmol, CAS #9003-05-8) in DMF (10 mL) was added tBuOK (1 M, 1.95 mL). The mixture was then stirred at 0° C. for 20 min. On completion, the reaction mixture was quenched by addition of 1M HCl (20 mL), and then extracted with EtOAc (30 mL×3). The combined organic layers were washed with brine (30 mL×3), dried over Na₂SO₄, filtered and concentrated under reduced pressure to give the title compound (610 mg) as a yellow solid. LC-MS (ESI⁺) m/z 419.1 (M+H)⁺.

### 4-(4-Chloro-2,5-difluorophenyl)piperidine (Intermediate ND)

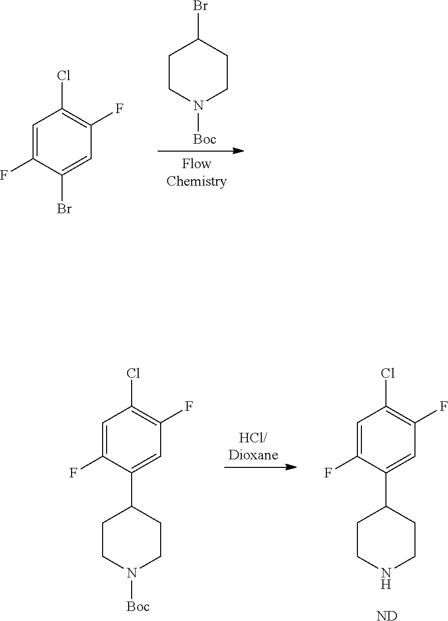 [SMILES: Fc1cc(Br)c(F)cc1Cl ; CC(C)(C)OC(=O)N1CCC(Br)CC1 ; CC(C)(C)OC(=O)N1CCC(c2cc(F)c(Cl)cc2F)CC1 ; Fc1cc(C2CCNCC2)c(F)cc1Cl ; "ND"]

### Step 1—Tert-butyl 4-(4-chloro-2,5-difluorophenyl)piperidine-1-carboxylate

A mixture of 1-bromo-4-chloro-2,5-difluorobenzene (3 g, 13 mmol, CAS #172921-33-4), tert-butyl 4-bromopiperidine-1-carboxylate (4.53 g, 17.1 mmol), TTMSS (3.94 g, 15.8 mmol, 4.88 mL), 2,6-dimethylpyridine (2.83 g. 26.3 mmol, 3.07 mL) and 4-tert-butyl-2-(4-tert-butyl-2-pyridyl)pyridine dichloronickel (52.5 mg, 131 μmol) in DME (250 mL) was degassed and purged with N₂ three times. Then the mixture was stirred at 40° C. for 40 mins under N₂ atmosphere (Flow chemistry). On completion, the reaction mixture was quenched with water (400 mL) and extracted with ethyl acetate (2×200 mL). The combined organic layers were washed with brine (400 mL) and dried over anhydrous sodium sulfate, filtered and the filtrate was concentrated in vacuo to give the crude residue. The crude residue was purified by reversed-phase HPLC (0.1% FA condition) to give the title compound (3 g, 69% yield) as a yellow oil. LC-MS (ESI⁺) m/z 276.1 (M−55)⁺.

### Step 2—4-(4-Chloro-2,5-difluorophenyl)piperidine

To a solution of tert-butyl 4-(4-chloro-2,5-difluorophenyl)piperidine-1-carboxylate (3 g, 9 mmol) in DCM (20 mL) was added HCl/dioxane (4 M, 2 mL). The mixture was then stirred at 25° C. for 5 hrs. On completion, the reaction was concentrated under reduced pressure to give the title compound (2 g, 95% yield) as yellow oil. LC-MS (ESI⁺) m/z 233.6 (M+H)⁺.

### 3-(4-(4-(4-Chloro-2,5-difluorophenyl)piperidin-1-yl)-3-fluorophenyl)piperidine-2,6-dione (Intermediate NE)

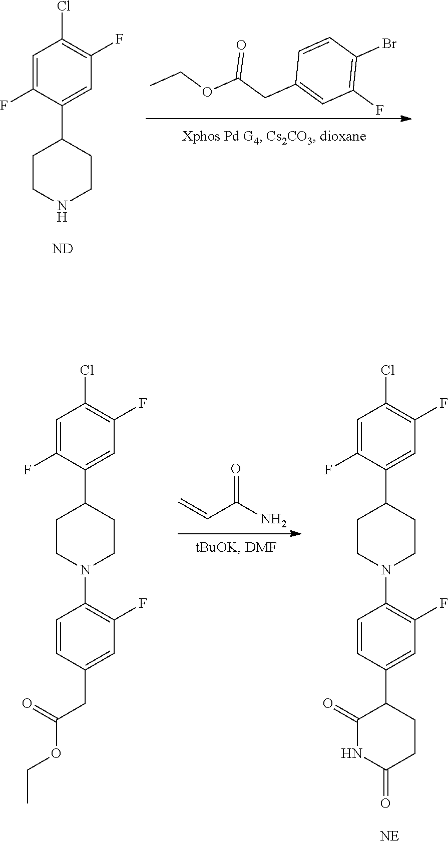

### Step 1—Ethyl 2-(4-(4-(4-chloro-2,5-difluorophenyl)piperidin-1-yl)-3-fluorophenyl)acetate

To a solution of 4-(4-chloro-2,5-difluorophenyl)piperidine (500 mg, 2.16 mmol, Intermediate ND) and ethyl 2-(4-bromo-3-fluorophenyl)acetate (845 mg, 3.24 mmol. CAS #1296223-82-9) in dioxane (5 mL) was added Xphos Pd G4 (186 mg, 216 μmol) and Cs₂CO₃ (2.11 g, 6.47 mmol) at 25° C. Then the mixture was stirred at 100° C. for 5 hrs. On completion, the reaction mixture was concentrated in vacuo to get the crude residue. The residue was purified by column chromatography (SiO₂, Petroleum ether/Ethyl acetate=30/1) to give the title compound (300 mg, 32% yield) as a white solid. LC-MS (ESI⁺) m/z 412.1 (M+1)⁺.

### Step 2—3-(4-(4-(4-Chloro-2,5-difluorophenyl)piperidin-1-yl)-3-fluorophenyl)piperidine-2,6-dione

To a solution of ethyl 2-(4-(4-(4-chloro-2,5-difluorophenyl)piperidin-1-yl)-3-fluorophenyl)acetate (300 mg, 728 μmol) and acrylamide (104 mg, 1.46 mmol, 100 mL, CAS #79-06-1) in DMF (5 mL) was added tBuOK (1 M, 801 μL) at 0° C., then the mixture was stirred at 25° C. for 10 min. On completion, the reaction mixture was added HCl (1N) until pH=3, the reaction mixture was quenched with water (20 mL) and extracted with ethyl acetate (3×20 mL). The extracts were filtered and concentrated in vacuo to give the title compound (200 mg) as a yellow solid. LC-MS (ESI⁺) m/z 436.9 (M+H)⁺.

### 3-(4-Bromo-3,3-dimethyl-2-oxoindolin-1-yl)piperidine-2,6-dione (Intermediate NF)

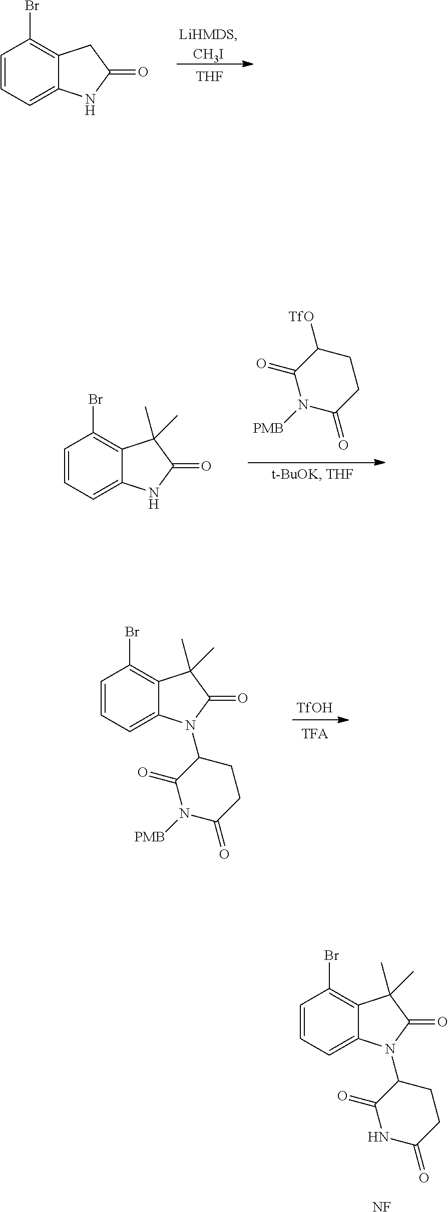

### Step 1—4-Bromo-3,3-dimethylindolin-2-one

To a solution of 4-bromoindolin-2-one (3 g, 14 mmol, CAS #99365-48-7) in THF (50 mL) was added dropwise LiHMDS (1 M, 56.6 mL) at −78° C. After addition, the mixture was stirred at this temperature for 1 hr, and then CH₃I (5.02 g. 35.4 mmol) was added dropwise at −78° C. The resulting mixture was stirred at 25° C. for 3 hrs. On completion, the reaction mixture was quenched with NH₄Cl (100 mL) and extracted with ethyl acetate (3×100 mL). The combined organic layers were washed with brine (2×80 mL) and dried over anhydrous sodium sulfate, filtered and the filtrate was concentrated in vacuo to give the title compound (3.2 g, 92% yield,) as a brown solid. LC-MS (ESI⁺) m/z 239.8 (M+H)⁺; ¹H NMR (400 MHz, DMSO-d₆) δ=10.59 (s, 1H), 7.15-7.07 (m, 2H), 6.91-6.83 (m, 1H), 1.36 (s, 6H).

### Step 2—3-(4-bromo-3,3-dimethyl-2-oxoindolin-1-yl)-1-(4-methoxybenzyl)piperidine-2,6-dione

To a solution of 4-bromo-3,3-dimethylindolin-2-one (1 g, 4.16 mmol) in THF (15 mL) was added t-BuOK (561 mg, 5.0 mmol) at 0° C. After addition, the mixture was stirred at this temperature for 1 hr, and then 1-(4-methoxybenzyl)-2,6-dioxopiperidin-3-yl trifluoromethanesulfonate (1.91 g, 5.0 mmol, CAS #2304754-47-8) in THF (5 mL) was added at 0° C. The resulting mixture was stirred at 25° C. for 2 hrs. On completion, the reaction mixture was quenched with NH4Cl (50 mL) and extracted with ethyl acetate (3×50 mL). The combined organic layers were washed with brine (2×50 mL) and dried over anhydrous sodium sulfate, filtered and the filtrate was concentrated in vacuo to give the residue. The residue was purified by column chromatography (SiO₂, Petroleum ether/Ethyl acetate=1/0 to 3/1) to give the title compound (1.6 g, 73% yield) as a white solid. LC-MS (ESI⁺) m/z 471.0 (M+H)⁺.

### Step 3—3-(4-Bromo-3,3-dimethyl-2-oxoindolin-1-yl)piperidine-2,6-dione

To a solution of 3-(4-bromo-3,3-dimethyl-2-oxoindolin-1-yl)-1-(4-methoxybenzyl)piperidine-2,6-dione (800 mg, 1.70 mmol) in TFA (6 mL) was added TfOH (2 mL). The mixture was then stirred at 60° C. for 3 hrs. On completion, the reaction mixture was diluted with water (50 mL) and extracted with ethyl acetate (3×50 mL). The combined organic layers were washed with brine (2×30 mL) and dried over anhydrous sodium sulfate, filtered and the filtrate was concentrated in vacuo to give the residue. The residue was purified by column chromatography (SiO₂, Petroleum ether/Ethyl acetate=1/0 to 1/1) to give the title compound (470 mg, 75% yield) as a yellow solid. LC-MS (ESI⁺) m/z 350.9 (M+H)⁺.

### 2-(1-(2,6-Dioxopiperidin-3-yl)-3,3-dimethyl-2-oxoindolin-4-yl)acetaldehyde (Intermediate NG)

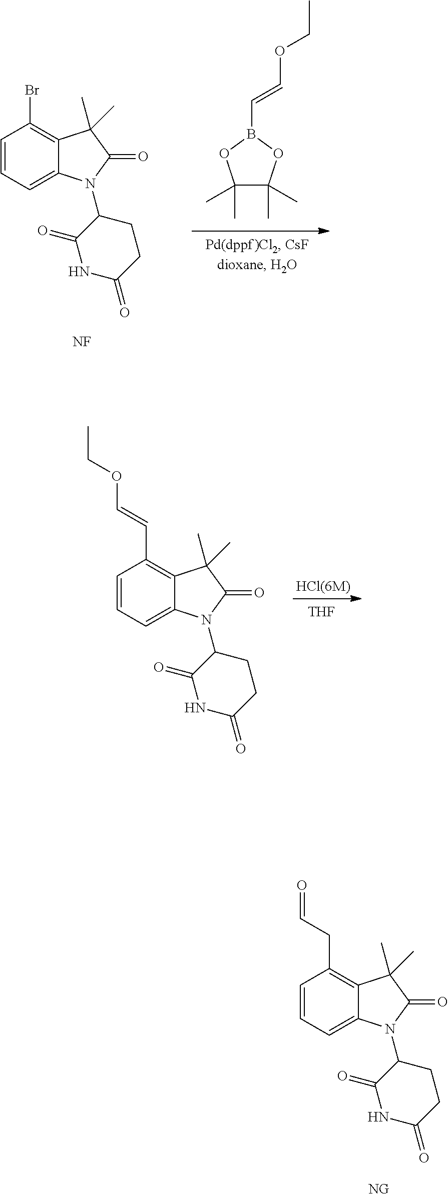

### Step 1—(E)-3-(4-(2-ethoxyvinyl)-3,3-dimethyl-2-oxoindolin-1-yl)piperidine-2,6-dione

A mixture of 3-(4-bromo-3,3-dimethyl-2-oxoindolin-1-yl)piperidine-2,6-dione (470 mg, 1.34 mmol, Intermediate NF), (E)-2-(2-ethoxyvinyl)-4,4,5,5-tetramethyl-1,3,2-dioxaborolane (265 mg, 1.34 mmol, CAS #1201905-21-4), Pd(dppf)Cl₂ (109 mg, 134 μmol), and CsF (609 mg, 4.01 mmol) in dioxane (8 mL) and H₂O (1.5 mL) was degassed and purged with N₂ three times. Then the mixture was stirred at 80° C. for 1 hr under N₂ atmosphere. On completion, the reaction mixture was concentrated in vacuo to give the residue. The residue was purified by column chromatography (SiO₂, Petroleum ether/Ethyl acetate=1/0 to 1/1) to give the title compound (350 mg. 71% yield) as a yellow solid. LC-MS (ESI⁺) m/z 343.0 (M+H)⁺.

### Step 2—2-(1-(2,6-Dioxopiperidin-3-yl)-3,3-dimethyl-2-oxoindolin-4-yl)acetaldehyde

To a solution of (E)-3-(4-(2-ethoxyvinyl)-3,3-dimethyl-2-oxoindolin-1-yl)piperidine-2,6-dione (300 mg, 876 μmol) in THF (5 mL) was added HCl (6 M, 5 mL). The mixture was stirred at 25° C. for 2 hrs. On completion, the reaction mixture was concentrated in vacuo to give the residue. The residue was purified by reversed-phase HPLC (0.1% FA condition) to give the title compound (200 mg, 72% yield) as a white solid. LC-MS (ESI⁺) m/z 314.9 (M+H)⁺.

### 3-[4-[(4R)-4-[4-[2,3-difluoro-4-(4,4,5,5-tetramethyl-1,3,2-dioxaborolan-2-yl)phenyl]piperazin-1-yl]-3,3-difluoro-1-piperidyl]-3-fluoro-phenyl]piperidine-2,6-dione (Intermediate NH)

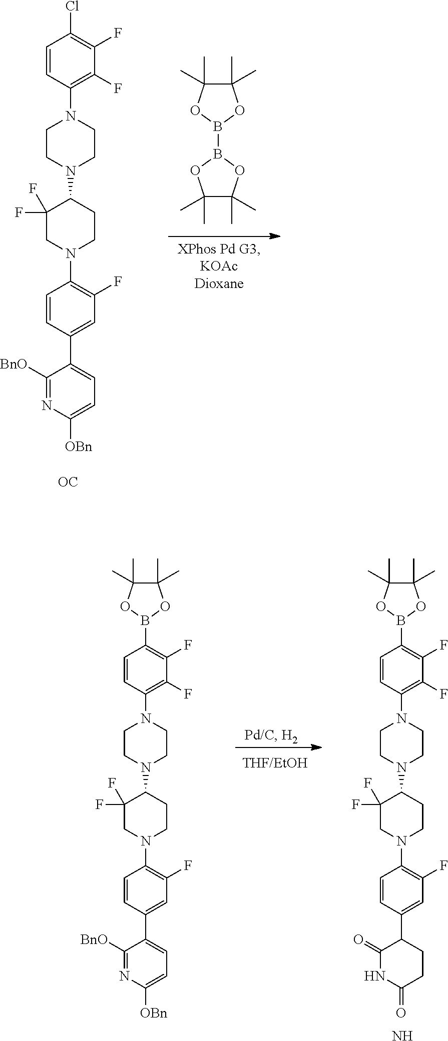

### Step 1 ((R)-1-(1-(4-(2,6-bis(benzyloxy)pyridin-3-yl)-2-fluorophenyl)-3,3-difluoropiperidin-4-yl)-4-(2,3-difluoro-4-(4,4,5,5-tetramethyl-1,3,2-dioxaborolan-2-yl)phenyl)piperazine

A mixture of 1-(4-chloro-2,3-difluoro-phenyl)-4-[(4R)-1-[4-(2,6-dibenzyloxy-3-pyridyl)-2-fluoro-phenyl]-3,3-difluoro-4-piperidyl]piperazine (400 mg, 544 μmol, Intermediate OC), 4,4,5,5-tetramethyl-2-(4,4,5,5-tetramethyl-1,3,2-dioxaborolan-2-yl)-1,3,2-dioxaborolane (276 mg, 1.09 mmol, CAS #73183-34-3), XPhos Pd G3 (69.0 mg, 81.6 μmol) and KOAc (160 mg, 1.63 mmol) in dioxane (5 mL) was degassed and purged with N₂ three times. Then the mixture was stirred at 80° C. for 12 hrs under N₂ atmosphere. On completion, the reaction mixture was concentrated under reduced pressure to give a residue. The residue was purified by column chromatography (SiO₂, Petroleum ether/Ethyl acetate=10/1 to 2/1) to give the title compound (0.4 g, 70% yield) as a gray solid. LC-MS (ESI⁺) m/z 827.3 (M+H)⁺.

### Step 2—3-(4-((R)-4-(4-(2,3-difluoro-4-(4,4,5,5-tetramethyl-1,3,2-dioxaborolan-2-yl)phenyl)piperazin-1-yl)-3,3-difluoropiperidin-1-yl)-3-fluorophenyl)piperidine-2,6-dione

To a solution of 1-[(4R)-1-[4-(2,6-dibenzyloxy-3-pyridyl)-2-fluoro-phenyl]-3,3-difluoro-4-piperidyl]-4-[2,3-difluoro-4-(4,4,5,5-tetramethyl-1,3,2-dioxaborolan-2-yl)phenyl]piperazine (200 mg, 241 μmol) in THE (3 mL) and EtOH (3 mL) was added Pd/C under N₂ atmosphere. The suspension was degassed and purged with H₂ for 3 times. The mixture was then stirred under H₂ (20 Psi) at 40° C. for 12 hrs. On completion, the reaction mixture was filtered and the filtrate was concentrated in vacuo to give the title compound (0.16 g) as a gray solid. LC-MS (ESI⁺) m/z 649.0 (M+H)⁺.

### 3-[4-[(4S)-4-[4-[2,3-difluoro-4-(4,4,5,5-tetramethyl-1,3,2-dioxaborolan-2-yl)phenyl]piperazin-1-yl]-3,3-difluoro-1-piperidyl]-3-fluoro-phenyl]piperidine-2,6-dione (Intermediate NI)

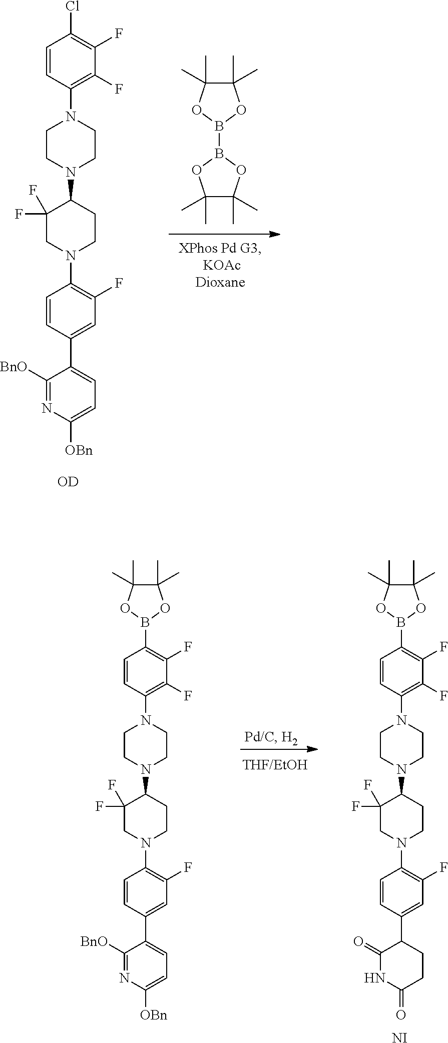

### Step 1—(S)-1-(1-(4-(2,6-bis(benzyloxy)pyridin-3-yl)-2-fluorophenyl)-3,3-difluoropiperidin-4-yl)-4-(2,3-difluoro-4-(4,4,5,5-tetramethyl-1,3,2-dioxaborolan-2-yl)phenyl)piperazine

A mixture of 1-(4-chloro-2,3-difluoro-phenyl)-4-[(4S)-1-[4-(2,6-dibenzyloxy-3-pyridyl)-2-fluoro-phenyl]-3,3-difluoro-4-piperidyl]piperazine (450 mg, 612 μmol, Intermediate OK), 4,4,5,5-tetramethyl-2-(4,4,5,5-tetramethyl-1,3,2-dioxaborolan-2-yl)-1,3,2-dioxaborolane (310 mg, 1.22 mmol, CAS #73183-34-3), XPhos Pd G3 (77.7 mg, 91.8 μmol), and KOAc (180 mg, 1.84 mmol) in dioxane (6 mL) was degassed and purged with N₂ three times. Then the mixture was stirred at 80° C. for 2 hrs under N₂ atmosphere. On completion, the reaction mixture was filtered and the filtrate was concentrated in vacuo to give the crude residue. The residue was purified by column chromatography (SiO₂, Petroleum ether/Ethyl acetate=10/1 to 2/1) to give the title compound (420 mg, 57% yield) as a gray solid. LC-MS (ESI⁺) m/z 827.2 (M+H)⁺.

### Step 2—3-(4-((S)-4-(4-(2,3-difluoro-4-(4,4,5,5-tetramethyl-1,3,2-dioxaborolan-2-yl)phenyl)piperazin-1-yl)-3,3-difluoropiperidin-1-yl)-3-fluorophenyl)piperidine-2,6-dione

To a solution of 1-[(4S)-1-[4-(2,6-dibenzyloxy-3-pyridyl)-2-fluoro-phenyl]-3,3-difluoro-4-piperidyl]-4-[2,3-difluoro-4-(4,4,5,5-tetramethyl-1,3,2-dioxaborolan-2-yl)phenyl]piperazine (254 mg, 308 μmol) in THF (3 mL), EtOH (3 mL) was added Pd/C under N₂ atmosphere. The suspension was degassed and purged with H, three times. The mixture was stirred under H, (20 Psi) at 40° C. for 12 hrs. On completion, the reaction mixture was filtered and the filtrate was concentrated in vacuo to give the title compound (160 mg) as a gray solid. LC-MS (ESI⁺) m/z 694.0 (M+H)⁺.

### 3-(4′-Bromo-2′-oxo-spiro[cyclopropane-1,3′-indoline]-1′-yl)piperidine-2,6-dione (Intermediate OH)

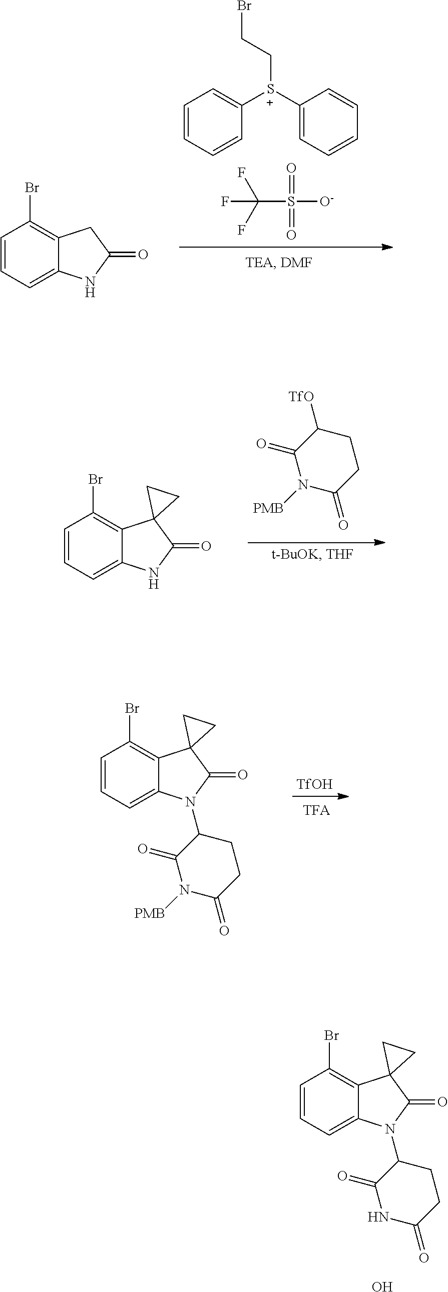

### Step 1—4′-Bromospiro[cyclopropane-1,3′-indoline]-2′-one

A mixture of 4-bromoindolin-2-one (1.5 g, 7.1 mmol, CAS #99365-48-7), 2-bromoethyl(diphenyl)sulfonium; trifluoromethanesulfonate (4.70 g, 10.6 mmol, CAS #247129-85-7) in DMF (15 mL) was degassed and purged with N₂ three times. Then TEA (2.15 g. 21.2 mmol. 2.95 mL) was added and the mixture was stirred at 25° C. for 6 hrs under N₂ atmosphere. On completion, the reaction mixture was diluted with water (100 mL) and extracted with ethyl acetate (100×2 mL). The combined organic layers were washed with brine (100 mL) and dried over anhydrous sodium sulfate, filtered and the filtrate was concentrated in vacuo to give the crude residue. The residue was purified by column chromatography (SiO₂, Petroleum ether:Ethyl acetate=1/0 to 5/1) to give the title compound (0.9 g, 53% yield) as a white solid. LC-MS (ESI⁺) m/z 237.9 (M+H)⁺; ¹H NMR (400 MHz, DMSO-d₆) δ=10.80 (br s, 1H), 7.14-7.04 (m, 2H), 6.93 (dd, J=1.4, 6.9 Hz, 1H), 2.11 (q, J=3.6 Hz, 2H), 1.35 (q, J=4.0 Hz, 2H).

### Step 2—3-(4′-Bromo-2′-oxo-spiro[cyclopropane-1,3′-indoline]-1′-yl)-1-[(4-methoxyphenyl)methyl]piperidine-2,6-dione

A mixture of 4′-bromospiro[cyclopropane-1,3′-indoline]-2′-one (850 mg, 3.57 mmol) in THF (10 mL) was added tBuOK (481 mg, 4.28 mmol) at 0° C. was degassed and purged with N₂ three times. After 1 hour, [1-[(4-methoxyphenyl)methyl]-2,6-dioxo-3-piperidyl]trifluoromethanesulfonate (1.63 g, 4.28 mmol) was added at 0° C., and then the mixture was stirred at 25° C. for 3 hrs under N₂ atmosphere. On completion, the reaction mixture was quenched with water (100 mL) and extracted with ethyl acetate (200×2 mL). The combined organic layers were washed with brine (100 mL) and dried over anhydrous sodium sulfate, filtered and the filtrate was concentrated in vacuo to give the crude residue. The residue was purified by column chromatography (SiO₂, Petroleum ether:Ethyl acetate=1/0 to 4/1) to give the title compound (1.3 g, 74% yield) as a white solid. LC-MS (ESI⁺) m/z 470.8 (M+H)f.

### Step 3—3-(4′-Bromo-2′-oxo-spiro[cyclopropane-1,3′-indoline]-1′-yl)piperidine-2,6-dione

A mixture of 3-(4′-bromo-2′-oxo-spiro[cyclopropane-1,3′-indoline]-1′-yl)-1-[(4-methoxyphenyl)methyl]piperidine-2,6-dione (600 mg, 1.28 mmol) in TFA (3 mL) and TfOH (1 mL) was degassed and purged with N₂ three times. Then the mixture was stirred at 60° C. for 12 hrs under N₂ atmosphere. On completion, the reaction mixture was diluted with water (50 mL) and extracted with ethyl acetate (100×2 mL). The combined organic layers were washed with brine (100 mL) and dried over anhydrous sodium sulfate, filtered and the filtrate was concentrated in vacuo to give the crude residue. The residue was purified by column chromatography (SiO₂, Petroleum ether:Ethyl acetate=1/0 to 1/1) to give the title compound (350 mg, 75% yield) as a yellow solid. LC-MS (ESI⁺) m/z 350.9 (M+H)⁺.

### 2-[1′-(2,6-Dioxo-3-piperidyl)-2′-oxo-spiro[cyclopropane-1,3′-indoline]-4′-yl]acetaldehyde (Intermediate NJ)

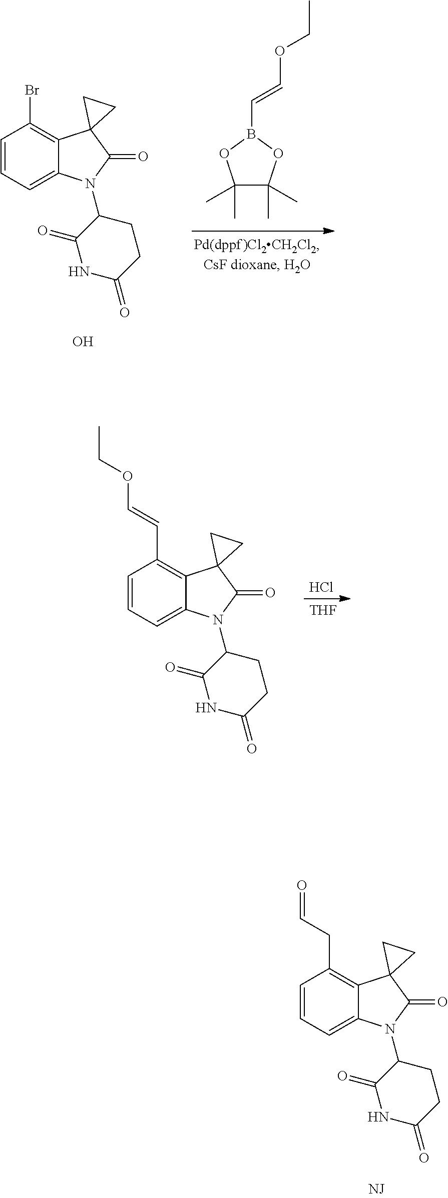

### Step 1—3-[4′-[(E)-2-ethoxyvinyl]-2′-oxo-spiro[cyclopropane-1,3′-indoline]-1′-yl]piperidine-2,6-dione

A mixture of 3-(4′-bromo-2′-oxo-spiro[cyclopropane-1,3′-indoline]-1′-yl)piperidine-2,6-dione (350 mg, 1.00 mmol, Intermediate OH), 2-[(E)-2-ethoxyvinyl]-4,4,5,5-tetramethyl-1,3,2-dioxaborolane (199 mg, 1.00 mmol, CAS #1201905-61-4), CsF (457 mg, 3.01 mmol), and Pd(dppf)Cl₂·CH₂Cl₂ (81.9 mg, 100 μmol) in dioxane (4 mL) and water (1 mL) was degassed and purged with N₂ three times. Then the mixture was stirred at 80° C. for 2 hrs under N₂ atmosphere. On completion, the mixture was concentrated in vacuo. The residue was purified by column chromatography (SiO₂, Petroleum ether:Ethyl acetate=1/0 to 1/1) to give the title compound (290 mg, 81% yield) as a white solid. LC-MS (ESI⁺) m/z 341.0 (M+H)⁺.

### Step 2—2-[1′-(2,6-Dioxo-3-piperidyl)-2′-oxo-spiro[cyclopropane-1,3′-indoline]-4′-yl]acetaldehyde

To a solution of 3-[4′-[(E)-2-ethoxyvinyl]-2′-oxo-spiro[cyclopropane-1,3′-indoline]-1′-yl]piperidine-2,6-dione (290 mg, 852 μmol) in THE (2 mL) was added HCl (6 M, 2 mL). The mixture was then stirred at 25° C. for 2 hrs. On completion, the crude residue was purified by reversed-phase HPLC (0.1% FA condition) to give the title compound (40 mg, 15% yield) as a white solid. LC-MS (ESI⁺) n/z 312.9 (M+H)⁺.

### 3-[4-[4-[(4R)-4-(4-bromo-2,3-difluoro-phenyl)-3,3-difluoro-1-piperidyl]-1-piperidyl]-3-fluoro-phenyl]piperidine-2,6-dione (Intermediate NK)

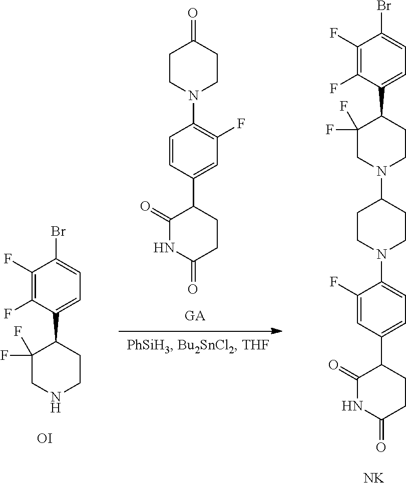

To a solution of (4R)-4-(4-bromo-2,3-difluoro-phenyl)-3,3-difluoro-piperidine (350 mg, 1.12 mmol, Intermediate OI) and 3-[3-fluoro-4-(4-oxo-1-piperidyl)phenyl]piperidine-2,6-dione (410 mg, 1.35 mmol, Intermediate GA) in THF (5 mL) was added phenylsilane (364 mg, 3.36 mmol) and dibutyl(dichloro)stannane (511 mg, 1.68 mmol). The mixture was then stirred at 60° C. for 6 hrs. On completion, the reaction mixture was quenched by addition of H₂O (30 mL), and then extracted with EA (30 mL×3). The combined organic layers were washed with brine (30 mL), dried over Na₂SO₄, filtered and concentrated under reduced pressure to give a residue. Then the crude product was purified by column chromatography (SiO₂, Petroleum ether/Ethyl acetate=2/1 to DCM/MeOH=2/1) to give the title compound (240 mg, 32% yield) as a white solid. LC-MS (ESI⁺) m/z 601.1 (M+H)⁺.

### 6-(1-(3-(1H-1,2,3-triazol-1-yl)propanoyl)-1,2,5,6-tetrahydropyridin-3-yl)-4-chloro-7-fluoro-1H-indole-2-carboxylic acid (Intermediate NL)

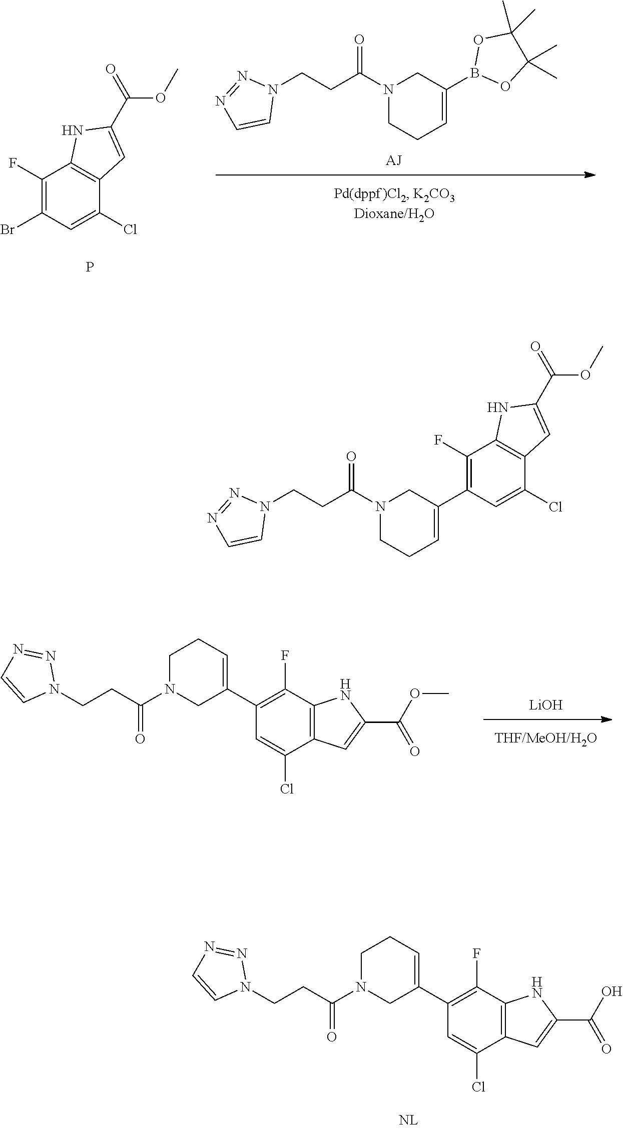

### Step 1—Methyl 6-(1-(3-(1H-1,2,3-triazol-1-yl)propanoyl)-1,2,5,6-tetrahydropyridin-3-yl)-4-chloro-7-fluoro-1H-indole-2-carboxylate (Intermediate Z)

To a solution of 1-(5-(4,4,5,5-tetramethyl-1,3,2-dioxaborolan-2-yl)-3,6-dihydropyridin-1 (2H)-yl)-3-(1H-1,2,3-triazol-1-yl)propan-1-one (700 mg, 2.11 mmol, Intermediate AJ), methyl 6-bromo-4-chloro-7-fluoro-1H-indole-2-carboxylate (710 mg, 2.32 mmol, Intermediate P), Pd(dppf)Cl₂ (154 mg, 211 μmol), and K₂CO₃ (874 mg. 6.32 mmol) in dioxane (6 mL) and H₂O (1.5 mL) was degassed and purged with N₂ three times. Then the mixture was stirred at 80° C. for 2 hrs under N₂ atmosphere. On completion, the reaction mixture was concentrated under reduced pressure to give a residue. The residue was purified by column chromatography (SiO₂, Petroleum ether/Ethyl acetate-5/1 to 0/1, DCM:MeOH=10:1) to give the title compound (400 mg, 41% yield) as a yellow solid. LC-MS (ESI⁺) m/z 432.1 (M+H)⁺.

### Step 2—6-(1-(3-(1H-1,2,3-triazol-1-yl)propanoyl)-1,2,5,6-tetrahydropyridin-3-yl)-4-chloro-7-fluoro-1H-indole-2-carboxylic acid

To a solution of methyl 6-(1-(3-(1H-1,2,3-triazol-1-yl)propanoyl)-1,2,5,6-tetrahydropyridin-3-yl)-4-chloro-7-fluoro-1H-indole-2-carboxylate (4 g, 9 mmol) in THF (20 mL), MeOH (5 mL) and H₂O (5 mL) was added LiOH·H₂O (1.17 g, 27.8 mmol). The mixture was then stirred at 25° C. for 4 h. On completion, the reaction mixture was quenched by addition of 6N HCl to adjust pH to 4-5, and then the mixture was extracted with EA (20 mL×3). The combined organic layers were washed with brine (30 mL), dried over Na₂SO₄, filtered and concentrated under reduced pressure to give the title compound (3 g, 54% yield) as a yellow solid. LC-MS (ESI⁺) m/z 418.0 (M+H)⁺.

### 6-(1-(3-(1H-1,2,3-triazol-1-yl)propanoyl)-1,2,5,6-tetrahydropyridin-3-yl)-4-chloro-N-ethyl-7-fluoro-N-methyl-1H-indole-2-carboxamide (Intermediate NM)

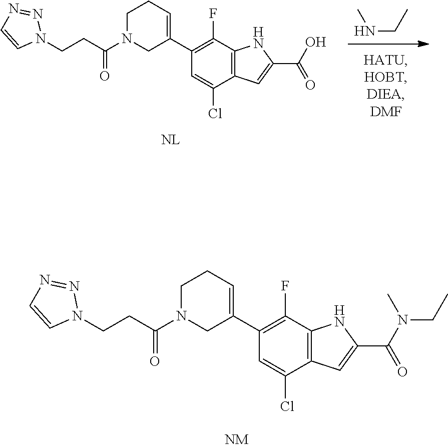

To a solution of 4-chloro-7-fluoro-6-[I-[3-(triazol-1-yl)propanoyl]-3,6-dihydro-2H-pyridin-5-yl]-1H-indole-2-carboxylic acid (100 mg, 239 μmol, Intermediate NL) in DMF (2 mL) was added dropwise HATU (137 mg, 359 μmol), HOBt (64.68 mg, 479 μmol) and DIEA (155 mg, 1.20 mmol) at 25° C. After addition, the mixture was stirred at 0° C. for 5 min, and then N-methylethanamine (17.0 mg, 287 μmol, CAS #624-78-2) was added dropwise at 0° C. The resulting mixture was stirred at 25° C. for 10 mins. On completion, the crude product was triturated with H₂O (15 mL) at 20° C. for 30 minutes, then the mixture was filtered to give the compound (60 mg, 55% yield) as a green solid. LC-MS (ESI⁺) m/z 459.1 (M+H)⁺.

### 2,6-bis(benzyloxy)-3-(4-(4-(4-chloro-3-fluorophenyl)piperidin-1-yl)-3-fluorophenyl)pyridine (Intermediate NN)

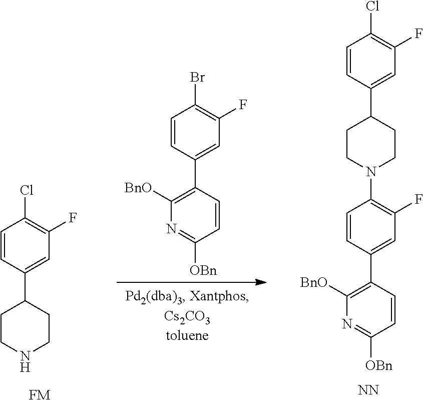

A mixture of 2,6-dibenzyloxy-3-(4-bromo-3-fluoro-phenyl)pyridine (3.5 g, 7.54 mmol, CAS #2902652-28-0), 4-(4-chloro-3-fluoro-phenyl)piperidine (1.93 g, 9.05 mmol, Intermediate FM), Pd₂(dba)₃ (690.25 mg, 753.78 μmol), Cs₂CO₃ (7.37 g, 22.6 mmol) and Xantphos (654.23 mg, 1.13 mmol) in toluene (10 mL) was degassed and purged with N₂ three times. Then the mixture was stirred at 100° C. for 12 hr under N₂ atmosphere. On completion, the reaction mixture was diluted with H₂O (100 mL) and extracted with EA (100 mL). The combined organic layers were washed with H₂O (200 mL), dried over Na₂SO₄, filtered and concentrated under reduced pressure to give a residue. The residue was purified by column chromatography (SiO₂, Petroleum ether/Ethyl acetate=30/1 to 10/1) to give title compound (2.2 g, 44% yield) as a yellow solid. LC-MS (ESI⁺) m/z 597.1 (M+H)f.

### 3-(3-Fluoro-4-(4-(3-fluoro-4-(4,4,5,5-tetramethyl-1,3,2-dioxaborolan-2-yl)phenyl)piperidin-1-yl)phenyl)piperidine-2,6-dione (Intermediate NO)

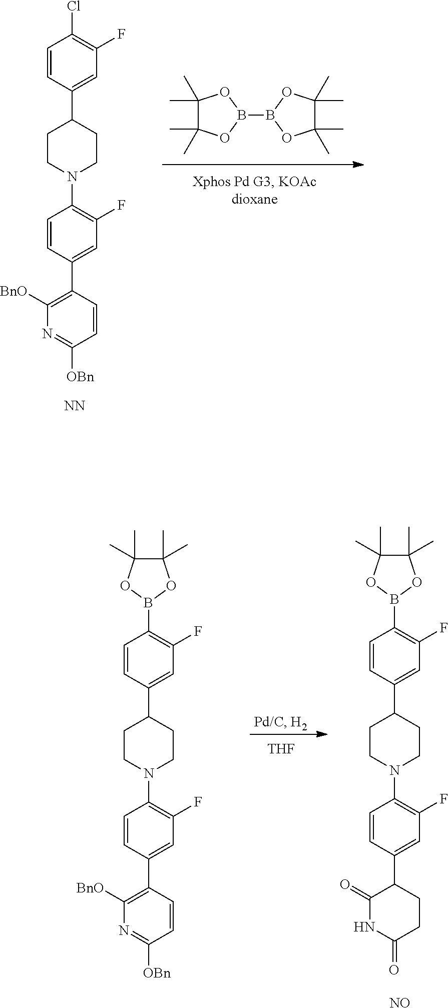

### Step 1—2,6-Bis(benzyloxy)-3-(3-fluoro-4-(4-(3-fluoro-4-(4,4,5,5-tetramethyl-1,3,2-dioxaborolan-2-yl)phenyl)piperidin-1-yl)phenyl)pyridine

A mixture of 2,6-dibenzyloxy-3-[4-[4-(4-chloro-3-fluoro-phenyl)-1-piperidyl]-3-fluoro-phenyl]pyridine (2.1 g, 3.5 mmol, Intermediate NN), 4,4,5,5-tetramethyl-2-(4,4,5,5-tetramethyl-1,3,2-dioxaborolan-2-yl)-1,3,2-dioxaborolane (1.07 g, 4.22 mmol), XPhos Pd G3 (297.70 mg, 351.70 μmol), and KOAc (1.04 g, 10.6 mmol) in dioxane (10 mL) was degassed and purged with N₂ three times. Then the mixture was stirred at 80° C. for 1 hr under N₂ atmosphere. On completion, the reaction mixture was diluted with H₂O (100 mL) and extracted with EA (100 mL). The combined organic layers were washed with H₂O (200 mL), dried over Na₂SO₄, filtered and concentrated under reduced pressure to give a residue. The residue was purified by column chromatography (SiO₂. Petroleum ether/Ethyl acetate=30/1 to 10/1) to give the title compound (1 g, 35% yield) as a red solid. LC-MS (ESI+) m/z 689.5 (M+H)⁺; ¹H NMR (400 MHz, DMSO-d6) δ=7.83-7.74 (m, 2H), 7.61 (td, J=7.0, 16.8 Hz, 2H), 7.47-7.33 (m, 13HT), 7.21-7.04 (m, 5H), 5.42 (s, 1H), 5.37 (s, 1H), 3.50 (br dd, J=3.7, 6.7 Hz, 1H), 3.23 (s, 1H), 3.18 (s, 1H), 1.29 (s, 12H), 1.16 (br s, 4H).

### Step 2—3-(3-Fluoro-4-(4-(3-fluoro-4-(4,4,5,5-tetramethyl-1,3,2-dioxaborolan-2-yl)phenyl)piperidin-1-yl)phenyl)piperidine-2,6-dione

To a solution of 2,6-dibenzyloxy-3-[3-fluoro-4-[4-[3-fluoro-4-(4,4,5,5-tetramethyl-1,3,2-dioxaborolan-2-yl)phenyl]-1-piperidyl]phenyl]pyridine (1 g, 2 mmol) in THF (50 mL) was added Pd/C (10 wt %, 1 g) under N₂ atmosphere. The suspension was degassed and purged with H₂ three times. The mixture was stirred under H₂ (40 Psi or atm.) at 30° C. for 12 hr. On completion, the reaction was filtered through kieselguhr very carefully, and the filtrated was concentrated in vacuo to give the title compound (500 mg, 68% yield) as a yellow solid. LC-MS (ESI+) m/z 511.2 (M+H)⁺.

### 3-[4-[4-(4-chloro-2,3-difluoro-phenyl)piperazin-1-yl]-3-fluoro-phenyl]piperidine-2,6-dione (Intermediate NP)

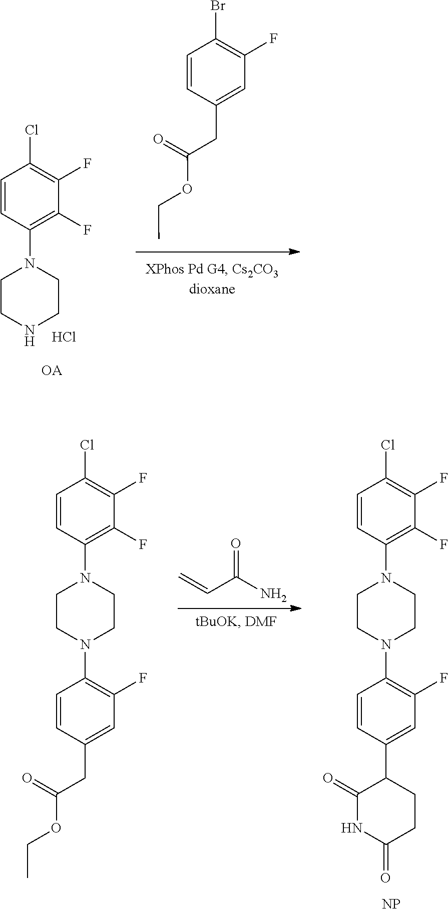

### Step 1—Ethyl 2-(4-(4-(4-chloro-2,3-difluorophenyl)piperazin-1-yl)-3-fluorophenyl)acetate

A mixture of 1-(4-chloro-2,3-difluoro-phenyl)piperazine (1.50 g, 6.45 mmol, Intermediate OA), ethyl 2-(4-bromo-3-fluoro-phenyl)acetate (1.68 g. 6.45 mmol, CAS #1296223-82-9). Xphos Pd G4 (1.11 g, 1.29 mmol), and Cs₂CO₃ (6.30 g, 19.3 mmol) in dioxane (10 mL) was degassed and purged with N₂ three times. Then the mixture was stirred at 100° C. for 1 hr under N₂ atmosphere. On completion, the reaction mixture was filtered and concentrated under reduced pressure to give a residue. The crude residue was purified by column chromatography (SiO₂, Petroleum ether/Ethyl acetate=30/1 to 10/1) to give the title compound (800 mg, 20% yield) as a white solid. LC-MS (ESI⁺) m/z 413.1 (M+H)⁺.

### Step 2—3-(4-(4-(4-Chloro-2,3-difluorophenyl)piperazin-1-yl)-3-fluorophenyl)piperidine-2,6-dione

To a solution of ethyl 2-[4-[4-(4-chloro-2,3-difluoro-phenyl)piperazin-1-yl]-3-fluoro-phenyl]acetate (800 mg, 1.94 mmol) and prop-2-enamide (152 mg, 2.13 mmol, CAS #9003-05-8) in DMF (6 mL) was added tBuOK (1 M, 2.33 mL). The mixture was stirred at 0° C. for 20 min. On completion, the reaction mixture was quenched by addition of 1M HCl (20 mL). The reaction mixture was filtered to give a filter cake which was dried to give the title compound (1 g, HCl) as a white solid. LC-MS (ESI⁺) m/z 438.1 (M+H)⁺.

### 5-Bromo-4-fluoro-2-iodopyridine (Intermediate NQ)

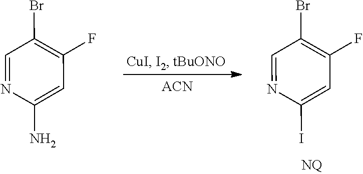

To a solution of 5-bromo-4-fluoro-pyridin-2-amine (7 g, 40 mmol, CAS #944401-69-8) in CH₃CN (100 mL) was added CuI (10.5 g, 55.0 mmol) and I₂ (18.6 g, 73.3 mmol, 14.8 mL), then tert-butyl nitrite (4.91 g, 47.6 mmol, 5.67 mL) was slowly added under N₂ at 0° C. The mixture was stirred at 0-60° C. for 2 hrs. On completion, the reaction mixture was partitioned between H₂O (10 mL). The organic phase was separated, washed with brine (10 mL×3), dried over Na₂SO₄, filtered and concentrated under reduced pressure to give a residue. The mixture was purified by column chromatography (SiO₂, Petroleum ether/Ethyl acetate=l/0 to 4/1) to give the title compound (3.03 g, 27% yield) as a yellow oil. LC-MS (ESI⁺) m/z 301.6 (M+H)⁺. ¹H NMR (400 MHz, DMSO-d₆) 5=1.08-1.26 (m, 3H) 1.99 (s, 3H) 4.03 (q, J=7.2 Hz, 2H) 7.98-8.13 (m, 1H) 8.64 (d, J=9.2 Hz. 1H)

### 2′,6′-Bis(benzyloxy)-5-bromo-4-fluoro-2,3′-bipyridine (Intermediate NR)

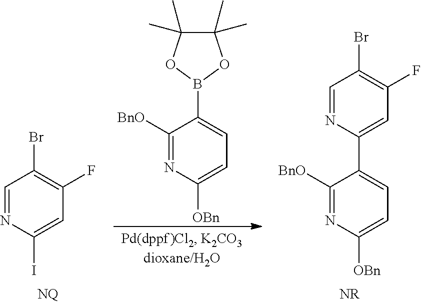 [SMILES: Fc1cc(I)ncc1Br ; CC1(C)OB(c2ccc(OCc3ccccc3)nc2OCc2ccccc2)OC1(C)C ; Fc1cc(-c2ccc(OCc3ccccc3)nc2OCc2ccccc2)ncc1Br]

A mixture of 5-bromo-4-fluoro-2-iodo-pyridine (2.7 g, 5.8 mmol, Intermediate NQ), 2,6-dibenzyloxy-3-(4,4,5,5-tetramethyl-1,3,2-dioxaborolan-2-yl)pyridine (2.43 g, 5.81 mmol, CAS #2152673-80-6), K₂CO₃ (1.61 g, 11.6 mmol), and Pd(dppf)Cl₂·CH₂Cl₂ (474 mg, 581 μmol) in dioxane (30 mL) and H₂O (10 mL) was degassed and purged with N₂ three times. Then the mixture was stirred at 60° C. for 12 hrs under N₂ atmosphere. On completion, the reaction mixture was concentrated in vacuo to give the crude residue. The residue was purified by column chromatography (SiO₂, Petroleum ether/Ethyl acetate=50/1 to 20/1) to give the title compound (1.2 g, 44% yield) as a white solid. LC-MS (ESI⁺) m/z 466.4 (M+H)⁺. ¹H NMR (400 MHz. DMSO-d₆) δ=5.47 (s, 2H) 5.58 (s, 2H) 6.66-6.71 (m, 1H) 7.36-7.47 (m, 10H) 8.05 (d, J=11.2 Hz, 1H) 8.44 (d, J=8.4 Hz, 1H) 8.90 (d, J=9.6 Hz, 1H).

### 2′-(Benzyloxy)-5-(4-(4-chlorophenyl)piperidin-1-yl)-4-fluoro-2,3′-bipyridine (Intermediate NS)

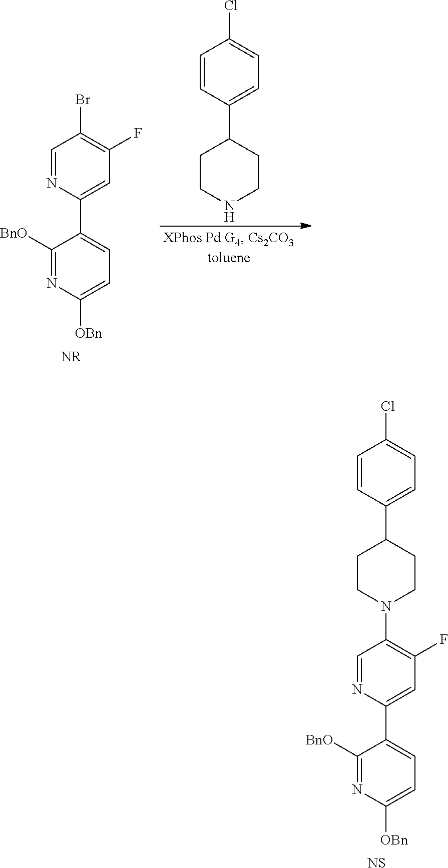

A mixture of 2,6-dibenzyloxy-3-(5-bromo-4-fluoro-2-pyridyl)pyridine (780 mg, 1.68 mmol. Intermediate NR), 4-(4-chlorophenyl)piperidine (328 mg, 1.68 mmol, CAS #26905-02-2). Cs₂CO₃ (1.37 g, 4.19 mmol), Pd₂(dba)₃ (107 mg, 117 μmol) and Xantphos (145 mg, 251 μmol) in toluene (10 mL) was degassed and purged with N₂ three times. Then the mixture was stirred at 100° C. for 12 hrs under N₂ atmosphere. On completion, the reaction mixture was concentrated in vacuo to give the crude residue. The residue was purified by column chromatography (SiO₂, Petroleum ether/Ethyl acetate=20/1 to 4/1) to give the title compound (530 mg, 55% yield) as a yellow solid. LC-MS (ESI⁺) m/z 580.4 (M+H)⁺.

### 3-(4-Fluoro-5-(4-(4-(4,4,5,5-tetramethyl-1,3,2-dioxaborolan-2-yl)phenyl)piperidin-1-yl)pyridin-2-yl)piperidine-2,6-dione (Intermediate NT)

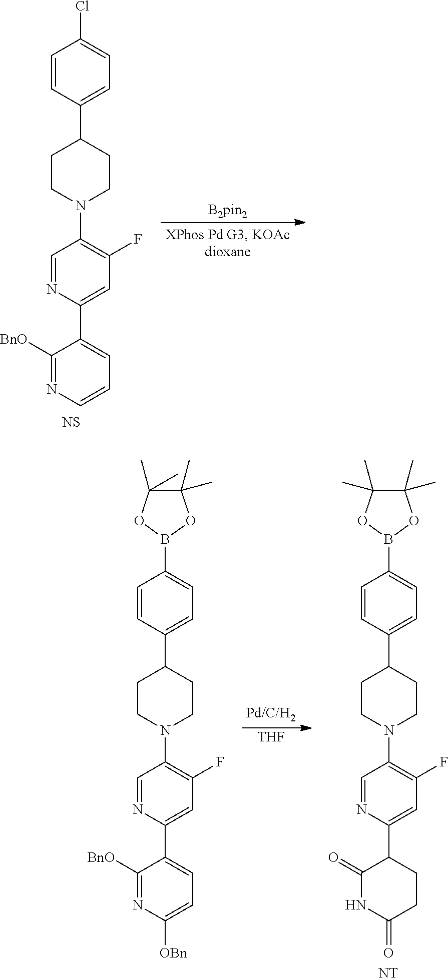

### Step 1—2′,6′-bis(benzyloxy)-4-fluoro-5-(4-(4-(4,4,5,5-tetramethyl-1,3,2-dioxaborolan-2-yl)phenyl)piperidin-1-yl)-2,3′-bipyridine

A mixture of 2,6-dibenzyloxy-3-[5-[4-(4-chlorophenyl)-1-piperidyl]-4-fluoro-2-pyridyl]pyridine (480 mg, 827 μmol, Intermediate NS), 4,4,5,5-tetramethyl-2-(4,4,5,5-tetramethyl-1,3,2-dioxaborolan-2-yl)-1,3,2-dioxaborolane (420 mg, 1.65 mmol, CAS #73183-34-3). KOAc (244 mg, 2.48 mmol), and XPhos Pd G3 (105 mg, 124 μmol) in dioxane (10 mL) was degassed and purged with N₂ three times. Then the mixture was stirred at 80° C. for 3 hrs under N₂ atmosphere. On completion, the reaction mixture was concentrated in vacuo to give the crude residue. The residue was purified by column chromatography (SiO₂, Petroleum ether/Ethyl acetate=20/1 to 5/1) to give the title compound (500 mg, 90% yield) as a yellow solid. LC-MS (ESI⁺) m/z 672.4 (M+H)⁺.

### Step 2—3-(4-Fluoro-5-(4-(4-(4,4,5,5-tetramethyl-1,3,2-dioxaborolan-2-yl)phenyl)piperidin-1-yl)pyridin-2-yl)piperidine-2,6-dione

To a solution of 2,6-dibenzyloxy-3-[4-fluoro-5-[4-[4-(4,4,5,5-tetramethyl-1,3,2-dioxaborolan-2-yl)phenyl]-1-piperidyl]-2-pyridyl]pyridine (250 mg, 372 μmol) in THE (5 mL) was added Pd/C (10 wt %, 0.48 g) under N₂ atmosphere. The suspension was degassed and purged with H₂ three times. The mixture was stirred under H₂ (40 Psi) at 30° C. for 48 hrs. On completion, the reaction was filtered through kieselguhr very carefully, the filtrated was concentrated in vacuo to give a residue. The residue was purified by column chromatography (SiO₂, Petroleum ether/Ethyl acetate=1/1) to give the title compound (164 mg, 89% yield) as a white solid. LC-MS (ESI⁺) m/z494.3 (M+H)⁺.

### Tert-butyl 4-(1-(5-bromo-3-fluoropyridin-2-yl)piperidin-4-yl)piperazine-1-carboxylate (Intermediate NU)

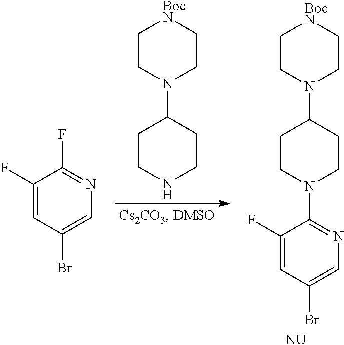

To a solution of tert-butyl 4-(4-piperidyl)piperazine-1-carboxylate (10 g, 40 mmol) 5-bromo-2,3-difluoro-pyridine (7.20 g, 37.1 mmol) in DMSO (100 mL) was added Cs₂CO₃ (36.29 g, 111.4 mmol). The mixture was then stirred at 120° C. for 12 hr. On completion, the mixture was extracted with EA (100 mL×3) and then concentrated under reduce pressure to give a residue. The residue was purified by column chromatography (SiO₂, Petroleum ether/Ethyl acetate=10/1 to 1/1) to give the title compound (19.2 g, 73% yield) as a yellow solid. LC-MS (ESI⁺) m/z 442.9 (M+H)⁺.

### 2,6-Bis(benzyloxy)-5′-fluoro-6′-(4-(piperazin-1-yl)piperidin-1-yl)-3,3′-bipyridine (Intermediate NV)

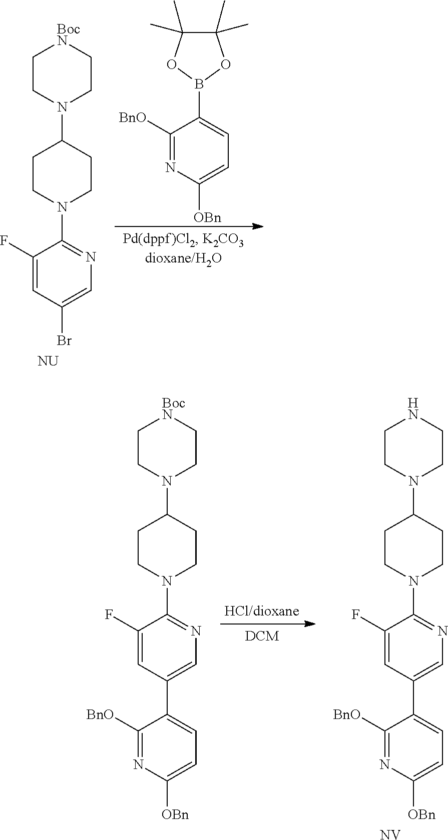

### Step 1—Tert-butyl 4-(1-(2′,6′-bis(benzyloxy)-5-fluoro-[3,3′-bipyridin]-6-yl)piperidin-4-yl)piperazine-1-carboxylate

A mixture of 2,6-dibenzyloxy-3-(4,4,5,5-tetramethyl-1,3,2-dioxaborolan-2-yl)pyridine (18.07 g, 43.31 mmol, synthesized via Step 1 of Intermediate AT), tert-butyl 4-[1-(5-bromo-3-fluoro-2-pyridyl)-4-piperidyl]piperazine-1-carboxylate (19.2 g, 43.3 mmol, Intermediate NU), Pd(dppf)Cl₂·CH₂Cl₂ (3.54 g, 4.33 mmol), K₂CO₃ (17.96 g, 129.9 mmol) in dioxane (200 mL) and H₂O (40 mL) was degassed and purged with N₂ three times. Then the mixture was stirred at 80° C. for 12 hr under N₂ atmosphere. On completion, the mixture was concentrated under reduce pressure to give a residue and purified by MPLC (SiO₂, PE:EA=10:1 to 0:1) to give the title compound (22 g, 67% yield) as a white solid. LC-MS (ESI⁺) m/z 654.3 (M+H)⁺.

### Step 2—2,6-Bis(benzyloxy)-5′-fluoro-6′-(4-(piperazin-1-yl)piperidin-1-yl)-3,3′-bipyridine

To a solution of tert-butyl 4-[1-[5-(2,6-dibenzyloxy-3-pyridyl)-3-fluoro-2-pyridyl]-4-piperidyl]piperazine-1-carboxylate (22 g, 33.6 mmol) in DCM (100 mL) was added HCl/EtOAc (8 M. 100 mL). The mixture was stirred at 25° C. for 1 hr. On completion, the mixture was concentrated under reduce pressure to give the title compound (20 g) as a yellow solid. LC-MS (ESF) m/z 554.1 (M+H)⁻.

### 2,6-Bis(benzyloxy)-6′-(4-(4-(4-chloro-2,3-difluorophenyl)piperazin-1-yl)piperidin-1-yl)-5′-fluoro-3,3′-bipyridine (Intermediate NW)

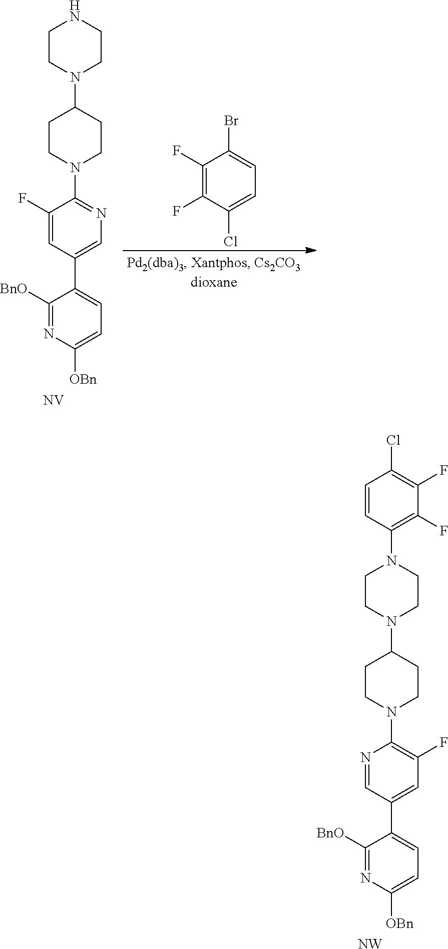 [SMILES: Fc1cc(-c2ccc(OCc3ccccc3)nc2OCc2ccccc2)cnc1N1CCC(N2CCNCC2)CC1 ; Fc1c(Cl)ccc(Br)c1F ; "NV" ; Fc1cc(-c2ccc(OCc3ccccc3)nc2OCc2ccccc2)cnc1N1CCC(N2CCN(c3ccc(Cl)c(F)c3F)CC2)CC1 ; "NW"]

A mixture of 1-bromo-4-chloro-2,3-difluoro-benzene (3.08 g, 13.5 mmol), 1-[1-[5-(2,6-dibenzyloxy-3-pyridyl)-3-fluoro-2-pyridyl]-4-piperidyl]piperazine (2.5 g, 4.5 mmol, Intermediate NV), Xantphos (522 mg, 903 μmol), Cs₂CO₃ (4.41 g, 13.5 mmol) and Pd₂(dba)₃ (413 mg, 451 μmol) in toluene (30 mL) was degassed and purged with N₂ three times. Then the mixture was stirred at 80° C. for 12 hrs under N₂ atmosphere. On completion, the mixture was concentrated under reduce pressure to give a residue and the mixture was purified by reversed-phase HPLC (0.1% FA) to give title compound (2.1 g, 55% yield) as a white solid. LC-MS (ESI⁺) m/z 700.2 (M+H)⁻.

### 3-(6-(4-(4-(2,3-Difluoro-4-(4,4,5,5-tetramethyl-1,3,2-dioxaborolan-2-yl)phenyl)piperazin-1-yl)piperidin-1-yl)-5-fluoropyridin-3-yl)piperidine-2,6-dione (Intermediate NX)

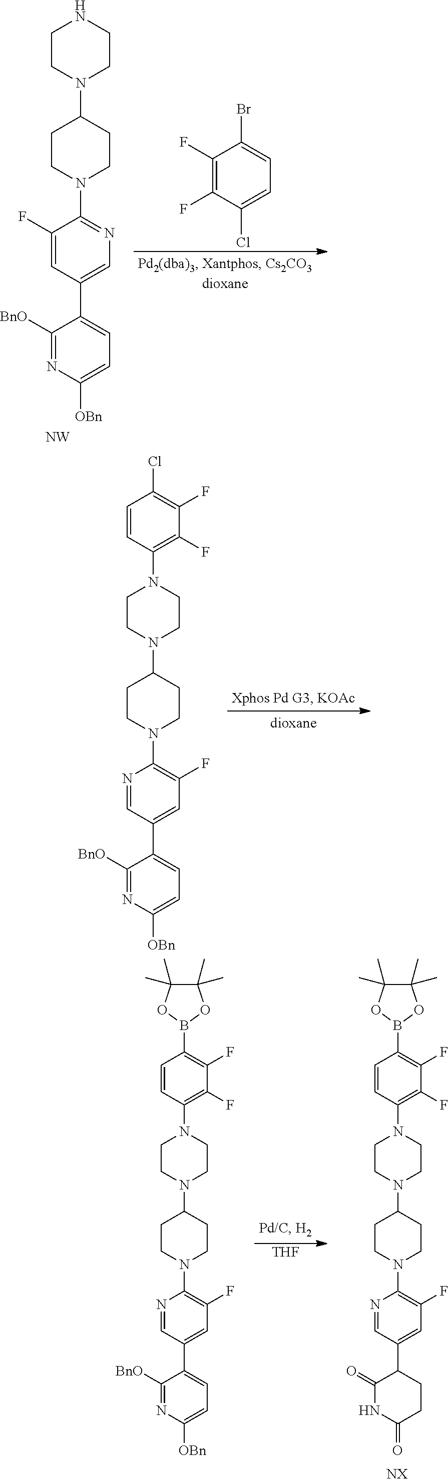

### Step 1—2,6-Bis(benzyloxy)-6′-(4-(4-(2,3-difluoro-4-(4,4,5,5-tetramethyl-1,3,2-dioxaborolan-2-yl)phenyl)piperazin-1-yl)piperidin-1-yl)-5′-fluoro-3,3′-bipyridine

A mixture of 1-(4-chloro-2,3-difluoro-phenyl)-4-[1-[5-(2,6-dibenzyloxy-3-pyridyl)-3-fluoro-2-pyridyl]-4-piperidyl]piperazine (2.05 g, 2.93 mmol, Intermediate NW), 4,4,5,5-tetramethyl-2-(4,4,5,5-tetramethyl-1,3,2-dioxaborolan-2-yl)-1,3,2-dioxaborolane (2.23 g, 8.78 mmol), XPhos Pd G3 (247.82 mg, 292.78 μmol), and KOAc (862.02 mg, 8.78 mmol) in dioxane (20 mL) was degassed and purged with N₂ three times. Then the mixture was stirred at 80° C. for 12 hr under N₂ atmosphere. On completion, the mixture was concentrated under reduce pressure to give a residue and purified by MPLC (SiO₂. PE:EA=10:1 to 0:1) to give the title compound (2 g. 67% yield) as a yellow solid. LC-MS (ESI⁺) m/z 792.4 (M+H)⁺.

### Step 2—3-(6-(4-(4-(2,3-Difluoro-4-(4,4,5,5-tetramethyl-1,3,2-dioxaborolan-2-yl)phenyl)piperazin-1-yl)piperidin-1-yl)-5-fluoropyridin-3-yl)piperidine-2,6-dione

A mixture of 1-[1-[5-(2,6-dibenzyloxy-3-pyridyl)-3-fluoro-2-pyridyl]-4-piperidyl]-4-[2,3-difluoro-4-(4,4,5,5-tetramethyl-1,3,2-dioxaborolan-2-yl)phenyl]piperazine (2 g. 3 mmol), and Pd/C (2 g, 4 mmol, 20 wt %) in THF (20 mL) was degassed and purged with H₂ three times. Then the mixture was stirred at 40° C. for 12 hr under H₂ atmosphere. On completion, the mixture was filtered and the solution was concentrated under reduced pressure to give title compound (1.3 g) as a white solid. LC-MS (ESI⁺) m/z 614.3 (M+H)⁺.

### 1-(2-Chloro-5-(methyl(piperidin-4-yl)amino)phenyl)dihydropyrimidine-2,4(1H,3H)-dione (Intermediate NY)

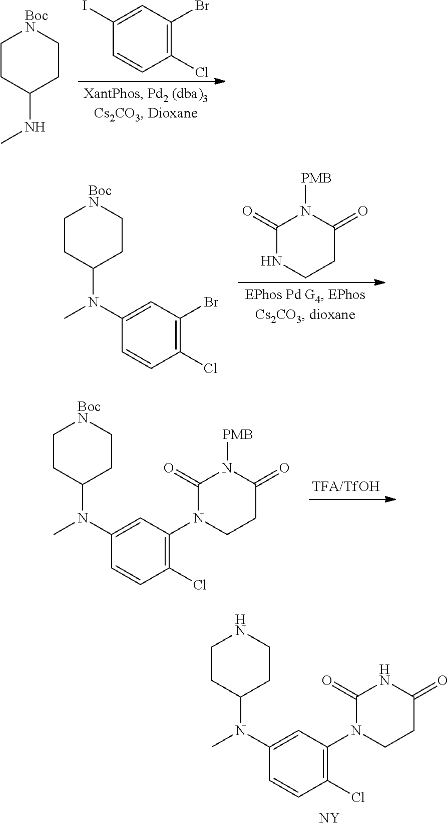

### Step 1—Tert-butyl 4-((3-bromo-4-chlorophenyl)(methyl)amino)piperidine-1-carboxylate

A mixture of 2-bromo-1-chloro-4-iodo-benzene (9.63 g, 30.3 mmol. CAS #31928-46-8), tert-butyl 4-(methylamino)piperidine-1-carboxylate (5 g, 20 mmol, CAS #147539-41-1), Cs₂CO₃ (22.8 g, 69.9 mmol), Pd₂(dba)₃ (1.07 g, 1.17 mmol) and Xantphos (1.35 g, 2.33 mmol) in toluene (40 mL) was degassed and purged with N₂ three times. Then the mixture was stirred at 80° C. for 12 hrs under N₂ atmosphere. On completion, the reaction mixture was concentrated under reduced pressure to give a residue. The residue was purified by column chromatography (SiO₂, Petroleum ether/Ethyl acetate=1/0 to 20/1) to give the title compound (3 g, 30% yield) as a yellow solid. LC-MS (ESI⁺) m/z 348.7 (M+H)⁺.

### Step 2—Tert-butyl 4-((4-chloro-3-(3-(4-methoxybenzyl)-2,4-dioxotetrahydropyrimidin-1 (2H)-yl)phenyl)(methyl)amino)piperidine-1-carboxylate

A mixture of tert-butyl 4-(3-bromo-4-chloro-N-methyl-anilino)piperidine-1-carboxylate (2.2 g, 5.5 mmol), 3-[(4-methoxyphenyl)methyl]hexahydropyrimidine-2,4-dione (1.91 g, 8.17 mmol, CAS #2631060-00-7), Cs₂CO₃ (8.88 g, 27.2 mmol), EPhos (227 mg, 544 μmol) and EPhos Pd G4 (501 mg, 544 μmol) in dioxane (30 mL) was degassed and purged with N₂ three times. Then the mixture was stirred at 100° C. for 72 hrs under N₂ atmosphere. On completion, the reaction mixture was filtered and the filtrate was concentrated in vacuo to give the crude residue. The residue was purified by column chromatography (SiO2, Petroleum ether/Ethyl acetate=10/1 to 5/1) to give the title compound (1 g, 29% yield) as a brown solid. LC-MS (ESI⁺) m/z 457.2 (M+H)⁺.

### Step 3—1-(2-Chloro-5-(methyl(piperidin-4-yl)amino)phenyl)dihydropyrimidine-2,4(H,3H)-dione

To a solution of tert-butyl 4-[4-chloro-3-[3-[(4-methoxyphenyl)methyl]-2,4-dioxo-hexahydropyrimidin-1-yl]-N-methyl-anilino]piperidine-1-carboxylate (900 mg, 1.62 mmol) was added TFA (13.8 g, 121 mmol) and TfOH (15.2 g, 101 mmol). The mixture was then stirred at 60° C. for 2 hrs. On completion, the reaction mixture was filtered and the filtrate was concentrated in vacuo to give the crude residue. The crude residue was purified by reversed-phase HPLC (0.1% FA condition) to give the title compound (500 mg, 80% yield) as a brown solid. LC-MS (ESI⁺) m/z 337.0 (M+H)⁺.

### 1-(5-((1′-(4-Bromophenyl)-[1,4′-bipiperidin]-4-yl)(methyl)amino)-2-chlorophenyl)dihydropyrimidine-2,4(1H,3H)-dione (Intermediate NZ)

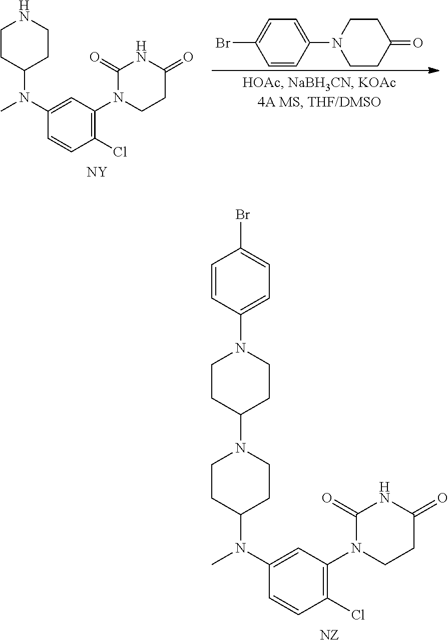

To a solution of 1-[2-chloro-5-[methyl(4-piperidyl)amino]phenyl]hexahydropyrimidine-2,4-dione (150 mg, 445 μmol, Intermediate NY) in THE (2 mL), DMSO (2 mL) was added KOAc (87.4 mg, 890 μmol) and 4 Å molecular sieves (200 mg). Then 1-(4-bromophenyl)piperidin-4-one (147 mg, 578 μmol, CAS #154913-23-2) and HOAc (80.2 mg, 1.34 mmol) was added and the mixture was stirred at 45° C. for 12 hrs. Next, NaBH₃CN (55.9 mg, 890 μmol) was added at 0° C., then the mixture was stirred at 60° C. for 12 hrs. On completion, the reaction mixture was concentrated under reduced pressure to give a residue. The crude residue was purified by reversed-phase HPLC (0.8 g/L ammonium bicarbonate) to give the title compound (45 mg, 15% yield) as a white solid. LC-MS (ESI⁺) m/z 576.0 (M+H)⁺.

### 1-(4-Chloro-2,3-difluorophenyl)piperazine hydrochloride (Intermediate OA)

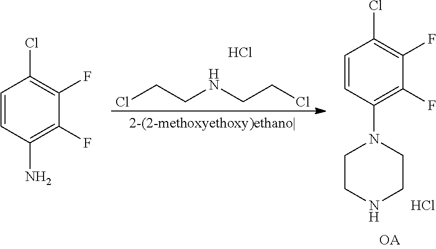

To a solution of 4-chloro-2,3-difluoro-aniline (15 g. 92 mmol, CAS #878285-12-2). 2-chloro-N-(2-chloroethyl)ethanamine (16.3 g, 91.7 mmol, HCl, CAS #334-22-5) in 2-(2-methoxyethoxy)ethanol (25 mL). The mixture was then stirred at 150° C. for 24 hrs. On completion, the reaction mixture was quenched by addition of acetone (100 mL). Then the reaction mixture was filtered and the filter cake was concentrated in vacuo to give the title compound (10.5 g) as an off-white solid. LC-MS (ESI⁺) m/z 233.1 (M+H)⁺; ¹H NMR (400 MHz, DMSO-d₆) δ=9.33 (br s, 2H), 7.42-7.29 (m, 1H), 6.98 (dt, J=2.0, 8.8 Hz, 1H), 3.32-3.27 (m, 4H), 3.26-3.20 (m, 4H).

### 1-(4-Chloro-2,3-difluorophenyl)-4-(3,3-difluoropiperidin-4-yl)piperazine (Intermediate OB)

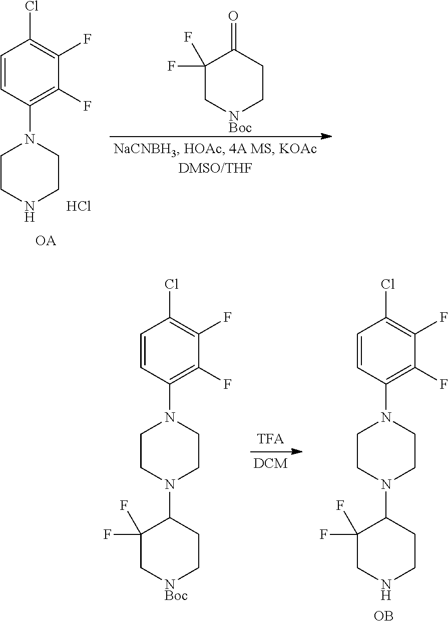

### Step 1—Tert-butyl 4-(4-(4-chloro-2,3-difluorophenyl)piperazin-1-yl)-3,3-difluoropiperidine-1-carboxylate

To a solution of 1-(4-chloro-2,3-difluoro-phenyl)piperazine (7 g, 26 mmol, HCl, Intermediate OA) in DMSO (50 mL) and THF (20 mL) was added 4 Å molecular sieves (10 g, 371 μmol) and KOAc (5.11 g. 52.2 mmol) until the pH was 6. Then tert-butyl 3,3-difluoro-4-oxo-piperidine-1-carboxylate (7.95 g, 33.8 mmol, CAS #1215071-17-2) and HOAc (4.69 g, 78.0 mmol) was added and the mixture was stirred at 50° C. for 12 hrs. Next, NaBH₃CN (3.27 g, 52.0 mmol) was added at 0° C., and the final mixture was stirred at 50° C. for 12 hrs. On completion, the reaction mixture was filtered and the filtrate was added water (150 mL) and extracted with ethyl acetate (3×150 mL). The combined organic layers were dried over anhydrous sodium sulfate, filtered and the filtrate was concentrated in vacuo to give the crude residue. The residue was purified by column chromatography (SiO₂, Petroleum ether/Ethyl acetate=6/1 to 4/1) to give the title compound (7 g, 30% yield) as a colorless oil. LC-MS (ESI⁺) m/z 452.0 (M+H)⁺.

### Step 2—1-(4-Chloro-2,3-difluorophenyl)-4-(3,3-difluoropiperidin-4-yl)piperazine

To a solution of tert-butyl 4-[4-(4-chloro-2,3-difluoro-phenyl)piperazin-1-yl]-3,3-difluoro-piperidine-1-carboxylate (7 g, 15 mmol) in DCM (20 mL) was added TFA (7.68 g, 67.3 mmol). The mixture was then stirred at 25° C. for 2 hrs. On completion, the reaction mixture was concentrated under reduced pressure to give a residue. The crude residue was purified by reversed-phase HPLC (0.8 g/L ammonium bicarbonate) to give the title compound (3 g, 45% yield) as a white solid. LC-MS (ESI⁺) m/z 352.0 (M+H)⁺.

### (R)-1-(1-(4-(2,6-bis(benzyloxy)pyridin-3-yl)-2-fluorophenyl)-3,3-difluoropiperidin-4-yl)-4-(4-chloro-2,3-difluorophenyl)piperazine (Intermediate OC) and (S)-1-(1-(4-(2,6-bis(benzyloxy)pyridin-3-yl)-2-fluorophenyl)-3,3-difluoropiperidin-4-yl)-4-(4-chloro-2,3-difluorophenyl)piperazine (Intermediate OD)

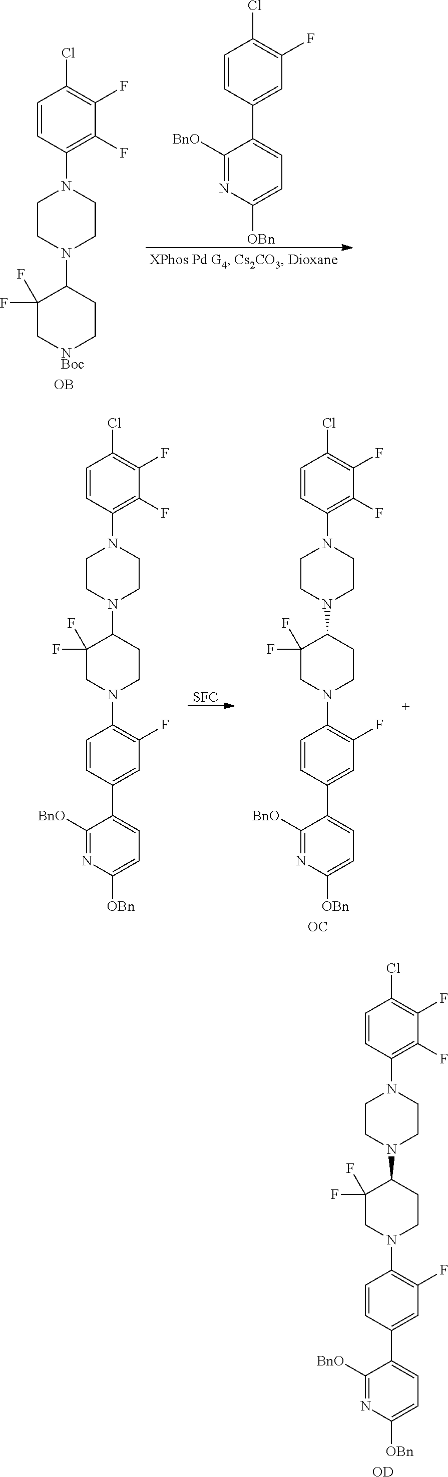

### Step 1—1-(1-(4-(2,6-Bis(benzyloxy)pyridin-3-yl)-2-fluorophenyl)-3,3-difluoropiperidin-4-yl)-4-(4-chloro-2,3-difluorophenyl)piperazine

A mixture of 1-(4-chloro-2,3-difluoro-phenyl)-4-(3,3-difluoro-4-piperidyl)piperazine (3 g, 9 mmol, Intermediate OB), 2,6-dibenzyloxy-3-(4-bromo-3-fluoro-phenyl)pyridine (3.96 g, 8.53 mmol, CAS #2902652-28-0), Cs₂CO₃ (8.34 g, 25.5 mmol), and Xphos Pd G4 (733 mg, 852 μmol) in dioxane (30 mL) was degassed and purged with N₂ three times. Then the mixture was stirred at 100° C. for 12 hrs under N₂ atmosphere. On completion, the reaction mixture was concentrated under reduced pressure to give a residue. The residue was purified by column chromatography (SiO₂, Petroleum ether/Ethyl acetate=5/1 to 3/1, DCM:EA=3:1) to give the title compound (1.1 g, 50% yield) as a white solid. LC-MS (ESI⁺) m/z 735.1 (M+H)⁺.

### Step 2—(R)-1-(1-(4-(2,6-bis(benzyloxy)pyridin-3-yl)-2-fluorophenyl)-3,3-difluoropiperidin-4-yl)-4-(4-chloro-2,3-difluorophenyl)piperazine and (S)-1-(1-(4-(2,6-bis(benzyloxy)pyridin-3-yl)-2-fluorophenyl)-3,3-difluoropiperidin-4-yl)-4-(4-chloro-2,3-difluorophenyl)piperazine

A solution of 1-(1-(4-(2,6-bis(benzyloxy)pyridin-3-yl)-2-fluorophenyl)-3,3-difluoropiperidin-4-yl)-4-(4-chloro-2,3-difluorophenyl)piperazine in EtOH (10 mL) was separated by column chromatography (column: DAICEL CHIRALPAK AD (250 mm×30 mm, 10 um); mobile phase: [CO2-ACN/i-PrOH (0.1% NH3H2O)]; B %:45%, isocratic elution mode) to give (R)-1-(1-(4-(2,6-bis(benzyloxy)pyridin-3-yl)-2-fluorophenyl)-3,3-difluoropiperidin-4-yl)-4-(4-chloro-2,3-difluorophenyl)piperazine (450 mg, 75% yield) as a white solid and (S)-1-(I-(4-(2,6-bis(benzyloxy)pyridin-3-yl)-2-fluorophenyl)-3,3-difluoropiperidin-4-yl)-4-(4-chloro-2,3-difluorophenyl)piperazine (450 mg, 75% yield) as a white solid. LC-MS (ESI⁺) m/z 735.2 (M+H)⁺ for both enantiomers. The absolute stereochemistry of the enantiomers was assigned arbitrarily.

### (S)-tert-butyl 4-(4-bromo-2,3-difluorophenyl)-3,3-difluoropiperidine-1-carboxylate (Intermediate OE) and (R)-tert-butyl 4-(4-bromo-2,3-difluorophenyl)-3,3-difluoropiperidine-1-carboxylate (Intermediate OF)

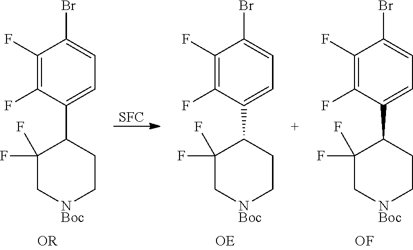

Tert-butyl 4-(4-bromo-2,3-difluorophenyl)-3,3-difluoropiperidine-1-carboxylate (Intermediate OR) was separated by column: DAICEL CHIRALPAK IG (250 mm×30 mm, 10 um); mobile phase: [CO2-EtOH (0.1% NH3H₂O)]:B %:10%, isocratic elution mode) to give (S)-tert-butyl 4-(4-bromo-2,3-difluorophenyl)-3,3-difluoropiperidine-1-carboxylate (350 mg, 100% purity) as a white solid and R tert-butyl 4-(4-bromo-2,3-difluorophenyl)-3,3-difluoropiperidine-1-carboxylate (370 mg, 100% purity) as a white solid. LC-MS (ESI⁺) m/z 356.0 (M−55)⁺ for both isomers. The absolute stereochemistry was assigned arbitrarily.

### (S)-4-(4-bromo-2,3-difluorophenyl)-3,3-difluoropiperidine (Intermediate OG)

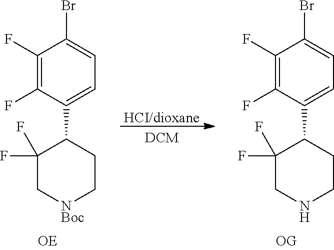

To a solution of tert-butyl (4S)-4-(4-bromo-2,3-difluoro-phenyl)-3,3-difluoro-piperidine-1-carboxylate (350 mg, 849 μmol, Intermediate OE) in DCM (4 mL) was added HCl/dioxane (8 M, 1 mL). The mixture was then stirred at 25° C. for 1 hr. On completion, the reaction mixture was concentrated under reduced pressure to give the title compound (360 mg, HCl) as a white solid. LC-MS (ESI⁺) m/z 311.7 (M+H)⁺.

### (R)-4-(4-bromo-2,3-difluorophenyl)-3,3-difluoropiperidine (Intermediate OI)

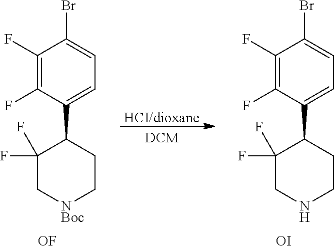

To a solution of tert-butyl (4R)-4-(4-bromo-2,3-difluoro-phenyl)-3,3-difluoro-piperidine-1-carboxylate (350 mg, 849 μmol, Intermediate OF) in DCM (4 mL) was added HCl/dioxane (8 M, 1 mL). The mixture was then stirred at 25° C. for 1 hr. On completion, the reaction mixture was concentrated under reduced pressure to give the title compound (370 mg, HCl) as a white solid. LC-MS (ESI⁺) m/z 311.7 (M+H)⁺.

### 4-(1-(5-Chloro-2-fluoro-4-iodophenyl)piperidin-4-yl)-5-fluoro-2-methoxyaniline (Intermediate OJ)

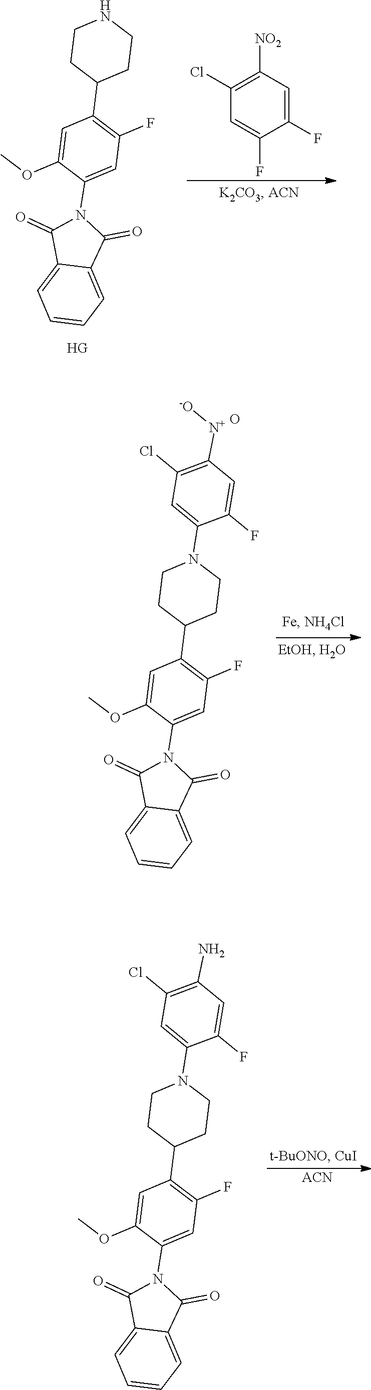

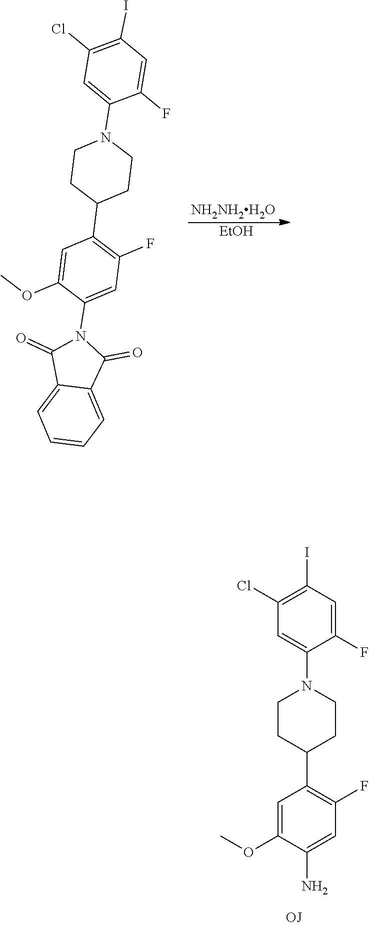

### Step 1—2-(4-(1-(5-Chloro-2-fluoro-4-nitrophenyl)piperidin-4-yl)-5-fluoro-2-methoxyphenyl)isoindoline-1,3-dione

To a solution of 1-chloro-4,5-difluoro-2-nitro-benzene (4.92 g, 25.4 mmol, CAS #771-76-6) and 2-(5-fluoro-2-methoxy-4-(piperidin-4-yl)phenyl)isoindoline-1,3-dione (9 g, 25 mmol, Intermediate HG) in DMF (100 mL) was added DIEA (9.85 g, 76.2 mmol). The mixture was stirred at 80° C. for 2 hs. On completion, the reaction mixture was filtered and the filter cake was concentrated in vacuo to give the crude product. The crude product was triturated with PE/EA=5:1 for 20 min, then the mixture was filtered and the filter cake was dried in vacuo to give the title compound (5 g, 37% yield) as a yellow solid. LC-MS (ESI⁺) m/z 528.0 (M+H)⁺; ¹H NMR (400 MHz, CHLOROFORM-d) δ=8.00-7.92 (m, 2H), 7.88-7.77 (m, 3H), 7.05-6.96 (m, 2H), 6.90 (d, J=6.0 Hz, 1H), 3.86 (d, J=12.0 Hz, 2H), 3.80 (s, 3H), 3.19-3.11 (m, 1H), 3.05 (t, J=11.2 Hz, 2H), 2.07-1.95 (m, 4H).

### Step 2—2-(4-(1-(4-Amino-5-chloro-2-fluorophenyl)piperidin-4-yl)-5-fluoro-2-methoxyphenyl)isoindoline-1,3-dione

To a solution of 2-(4-(I-(5-chloro-2-fluoro-4-nitrophenyl)piperidin-4-yl)-5-fluoro-2-methoxyphenyl)isoindoline-1,3-dione (5 g, 10 mmol) in EtOH (60 mL) and H₂O (60 mL) was degassed and purged with N₂ three times. Then to the mixture was added Fe (4.23 g, 75.8 mmol) and NH₄Cl (4.05 g, 75.8 mmol) at 25° C. The mixture was next stirred at 80° C. for 12 hrs under N₂ atmosphere. On completion, the reaction mixture was filtered, then the filtrate diluted with water (100 mL) and extracted with ethyl acetate (3×100 mL). The combined organic layers were washed with brine (2×100 mL) and dried over anhydrous sodium sulfate, filtered and the filtrate was concentrated in vacuo to give the title compound (4 g) as a yellow solid. LC-MS (ESI⁺) m/z 498.1 (M+H)⁺.

### Step 3—2-(4-(I-(5-Chloro-2-fluoro-4-iodophenyl)piperidin-4-yl)-5-fluoro-2-methoxyphenyl)isoindoline-1,3-dione

To a solution of 2-(4-(1-(4-amino-5-chloro-2-fluorophenyl)piperidin-4-yl)-5-fluoro-2-methoxyphenyl)isoindoline-1,3-dione (3.8 g, 7.6 mmol) in ACN (100 mL) was degassed and purged with N₂ three times. Then to the mixture was added tert-butyl nitrite (1.18 g, 11.4 mmol) and CuI (1.89 g, 9.92 mmol) and the mixture was stirred at 60° C. for 4 hrs under N₂ atmosphere. On completion, the reaction mixture was concentrated in vacuo to give the crude product. The crude product was triturated with DCM (200 ml), then the mixture was filtered and the filtrate was concentrated in vacuo to give the title compound (4 g, 24% yield) as a brown solid. LC-MS (ESI⁺) m/z 608.9 (M+H)⁺.

### Step 4—4-(1-(5-Chloro-2-fluoro-4-iodophenyl)piperidin-4-yl)-5-fluoro-2-methoxyaniline

To a solution of 2-(4-(1-(5-chloro-2-fluoro-4-iodophenyl)piperidin-4-yl)-5-fluoro-2-methoxyphenyl)isoindoline-1,3-dione (4 g, 7 mmol) in EtOH (50 mL) was degassed and purged with N₂ three times, and then to the mixture was added NH₂NH₂·H₂O (6.19 g, 105 mmol, 85% solution) at 25° C. The mixture was then stirred at 50° C. for 4 hrs under N₂ atmosphere. On completion, the reaction mixture was quenched by addition of H₂O (100 mL), and then extracted with EA (100 mL×3). The combined organic layers were washed with brine (100 mL), dried over Na₂SO₄, filtered and the filtrate concentrated under reduced pressure to give a residue. The residue was purified by column chromatography (SiO₂, Petroleum ether/Ethyl acetate=1/0 to 5/1) to give the title compound (700 mg, 20% yield) as a yellow solid. LC-MS (ESI⁺) m/z 478.8 (M+H)⁺; ¹H NMR (400 MHz, CHLOROFORM-d) δ=7.46 (d, J=11.6 Hz, 1H), 7.05 (d, J=8.0 Hz, 1H), 6.64 (d, J=6.8 Hz, 1H), 6.43 (d, J=11.2 Hz, 1H), 3.83 (s, 3H), 3.81 (s, 2H), 3.54 (d, J=11.6 Hz, 2H), 2.98-2.87 (m, 1H), 2.85-2.75 (m, 2H), 2.00-1.87 (m, 4H).

### 3-((4-(1-(5-Chloro-2-fluoro-4-iodophenyl)piperidin-4-yl)-5-fluoro-2-methoxyphenyl)amino)piperidine-2,6-dione (Intermediate OK)

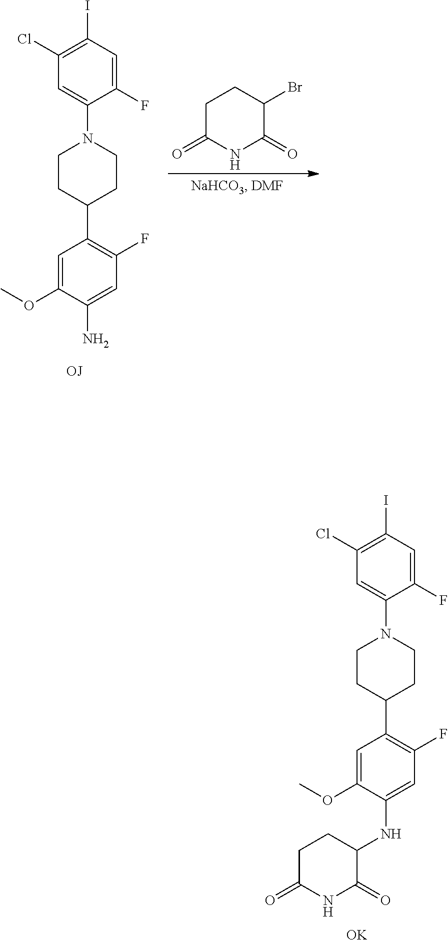

To a solution of 4-(1-(5-chloro-2-fluoro-4-iodophenyl)piperidin-4-yl)-5-fluoro-2-methoxyaniline (700 mg, 1.46 mmol, Intermediate OJ) in DMF (20 mL) was added NaHCO₃ (614 mg, 7.31 mmol) and 3-bromopiperidine-2,6-dione (842 mg, 4.39 mmol, CAS #62595-74-8). The mixture was stirred at 80° C. for 12 hrs. On completion, the reaction mixture was quenched with water (50 mL) and extracted with ethyl acetate (3×50 mL). The combined organic layers were washed with brine (2×50 mL) and dried over anhydrous sodium sulfate, filtered and the filtrate was concentrated in vacuo to give the crude product. The crude product was triturated with (PE/EA=1:1) at 25° C. for 0.5 hrs, then the mixture was filtered and the filter cake was dried in vacuo to give the title compound (500 mg, 46% yield) as a gray solid. LC-MS (ESI⁺) m/z 589.9 (M+H)⁺.

### 3-(4-(4-(4-Chlorophenyl)piperidin-1-yl)-3-fluorophenoxy)piperidine-2,6-dione (Intermediate OL)

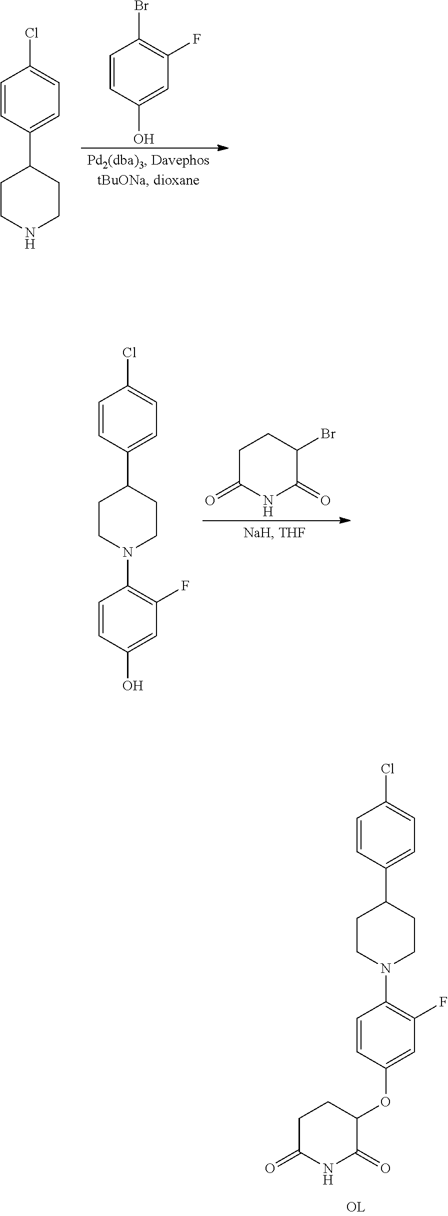 [SMILES: Clc1ccc(C2CCNCC2)cc1 ; Oc1ccc(Br)c(F)c1 ; Oc1ccc(N2CCC(c3ccc(Cl)cc3)CC2)c(F)c1 ; O=C1CCC(Br)C(=O)N1 ; O=C1CCC(Oc2ccc(N3CCC(c4ccc(Cl)cc4)CC3)c(F)c2)C(=O)N1]

### Step 1—4-(4-(4-Chlorophenyl)piperidin-1-yl)-3-fluorophenol

To a solution of 4-(4-chlorophenyl)piperidine (3.84 g, 19.6 mmol, CAS #26905-02-2) and 4-bromo-3-fluorophenol (2.5 g, 13 mmol, CAS #121219-03-2) in dioxane (25 mL) was added DavePhos (772 mg, 1.96 mmol), t-BuONa (3.77 g, 39.2 mmol) and Pd₂(dba)₃ (599 mg, 654 μmol) at 25° C. under nitrogen atmosphere. Then the mixture was stirred at 100° C. for 2 hrs under nitrogen atmosphere. On completion, the reaction mixture was quenched with NH4Cl (25 mL) and extracted with dichloromethane (3×20 mL). The combined organic layers were washed with brine (30 mL) and dried over anhydrous sodium sulfate, filtered and the filtrate was concentrated in vacuo to give the crude residue. The residue was purified by column chromatography (SiO₂, Petroleum ether/Ethyl acetate=5/1) to give the title compound (1.2 g, 23% yield) as a yellow solid. LC-MS (ESI⁺) m/z 303.7 (M+H)⁺.

### Step 2—3-(4-(4-(4-Chlorophenyl)piperidin-1-yl)-3-fluorophenoxy)piperidine-2,6-dione

To a solution of 4-(4-(4-chlorophenyl)piperidin-1-yl)-3-fluorophenol (1.1 g, 3.6 mmol) in THF (10 mL) was added NaH (359 mg, 8.99 mmol, 60% dispersion in mineral oil) at 0° C., then the mixture was stirred at 0° C. for 1 hr. Next, 3-bromopiperidine-2,6-dione (759 mg, 3.96 mmol, CAS #62595-74-8) in THF (5 mL) was added dropwise at 0° C., and the mixture was stirred at 25° C. for 2 hrs. On completion, the reaction mixture was quenched with NH4Cl (20 mL) and extracted with ethyl acetate (3×15 mL) The combined organic layers were washed with brine (15 mL) and dried over anhydrous sodium sulfate, filtered and the filtrate was concentrated in vacuo to give the crude residue. The residue was purified by column chromatography (SiO₂, Petroleum ether/Ethyl acetate=8/1 to 1.5/1) to give the title compound (640 mg, 39% yield) as a white solid. LC-MS (ESI⁺) m/z 417.1 (M+H)⁺.

### 2-(4-((2,6-Dioxopiperidin-3-yl)amino)-2-fluoro-5-methoxyphenyl)acetaldehyde (Intermediate OM)

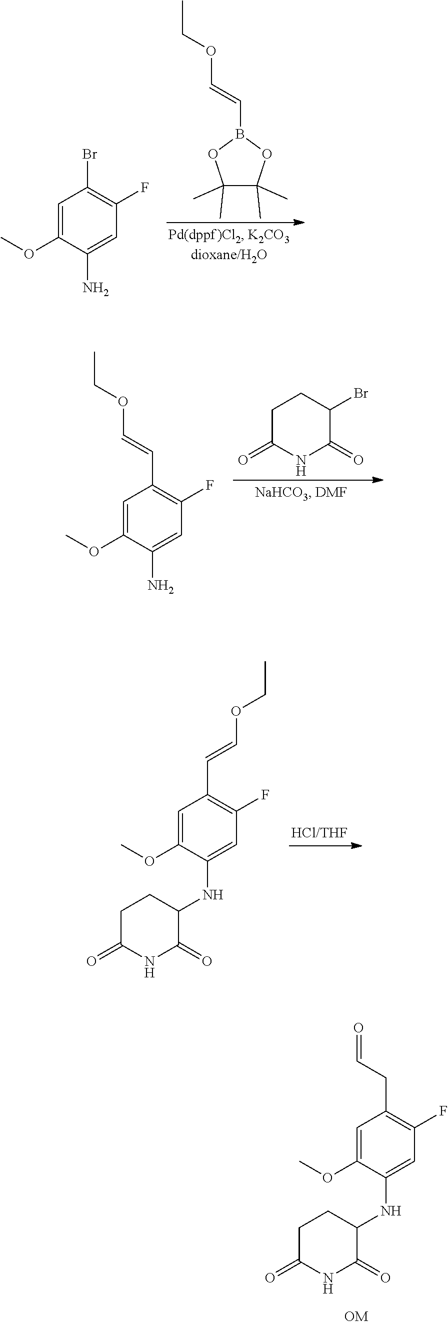

### Step 1—(E)-4-(2-ethoxyvinyl)-5-fluoro-2-methoxyaniline

A mixture of 4-bromo-5-fluoro-2-methoxyaniline (3 g, 14 mmol, CAS #30794-03-1), (E)-2-(2-ethoxyvinyl)-4,4,5,5-tetramethyl-1,3,2-dioxaborolane (2.70 g, 13.6 mmol, CAS #1201905-61-4), Pd(dppf)Cl₂ (998 mg, 1.36 mmol), and K₂CO₃ (1.88 g, 13.63 mmol) in dioxane (10 mL) and H₂O (2 mL) was degassed and purged with N₂ three times. Then the mixture was stirred at 80° C. for 2 hrs under N₂ atmosphere. On completion, the reaction mixture was quenched with water (30 mL) and extracted with EA (3×30 mL). The combined organic layers were washed with aqueous NaCl (30 mL) and dried over anhydrous sodium sulfate, filtered and the filtrate was concentrated in vacuo to give the crude residue. The residue was purified by column chromatography (SiO₂, Petroleum ether:Ethyl acetate=10/1) to give the title compound (2.49 g, 87% yield) as a yellow solid. LC-MS (ESI⁺) m/z 212.2 (M+H)⁺.

### Step 2—(E)-3-((4-(2-ethoxyvinyl)-5-fluoro-2-methoxyphenyl)amino)piperidine-2,6-dione

To a solution of (E)-4-(2-ethoxyvinyl)-5-fluoro-2-methoxyaniline (2.49 g, 8.24 mmol) 3-bromopiperidine-2,6-dione (3.16 g, 16.5 mmol, CAS #62595-74-8) in DMF (30 mL) was added NaHCO₃ (3.46 g, 41.2 mmol, 1.60 mL). The mixture was then stirred at 80° C. for 16 hrs. On completion, the reaction mixture was quenched with water (30 mL) and extracted with EA (3×30 mL). The combined organic layers were washed with aqueous NaCl (30 mL) and dried over anhydrous sodium sulfate, filtered and the filtrate was concentrated in vacuo to give the crude residue. The residue was purified by column chromatography (SiO₂, Petroleum ether:Ethyl acetate=3/1) to give the title compound (1.7 g, 64% yield) as a brown solid. LC-MS (ESI⁺) m/z 323.0 (M+H)f.

### Step 3—2-(4-((2,6-Dioxopiperidin-3-yl)amino)-2-fluoro-5-methoxyphenyl)acetaldehyde

A mixture of (E)-3-((4-(2-ethoxyvinyl)-5-fluoro-2-methoxyphenyl)amino)piperidine-2,6-dione (800 mg, 2.48 mmol) and HCl (6 M, 414 KL) in THE (2 mL) was degassed and purged with N₂ three times. Then the mixture was stirred at 80° C. for 2 hrs under N₂ atmosphere. On completion, the reaction mixture was concentrated in vacuo to give the crude residue. The crude residue was purified by reversed-phase HPLC (0.8 g/L ammonium bicarbonate) to give the title compound (300 mg, 41% yield) as a white solid. LC-MS (ESI⁺) m/z 295.0 (M+H)⁺.

### 6-(1-(3-(1H-pyrazol-1-yl)propanoyl)-1,2,5,6-tetrahydropyridin-3-yl)-7-fluoro-4-(2-fluoro-4-(piperazin-1-yl)phenyl)-N,N-dimethyl-1H-indole-2-carboxamide (Intermediate ON)

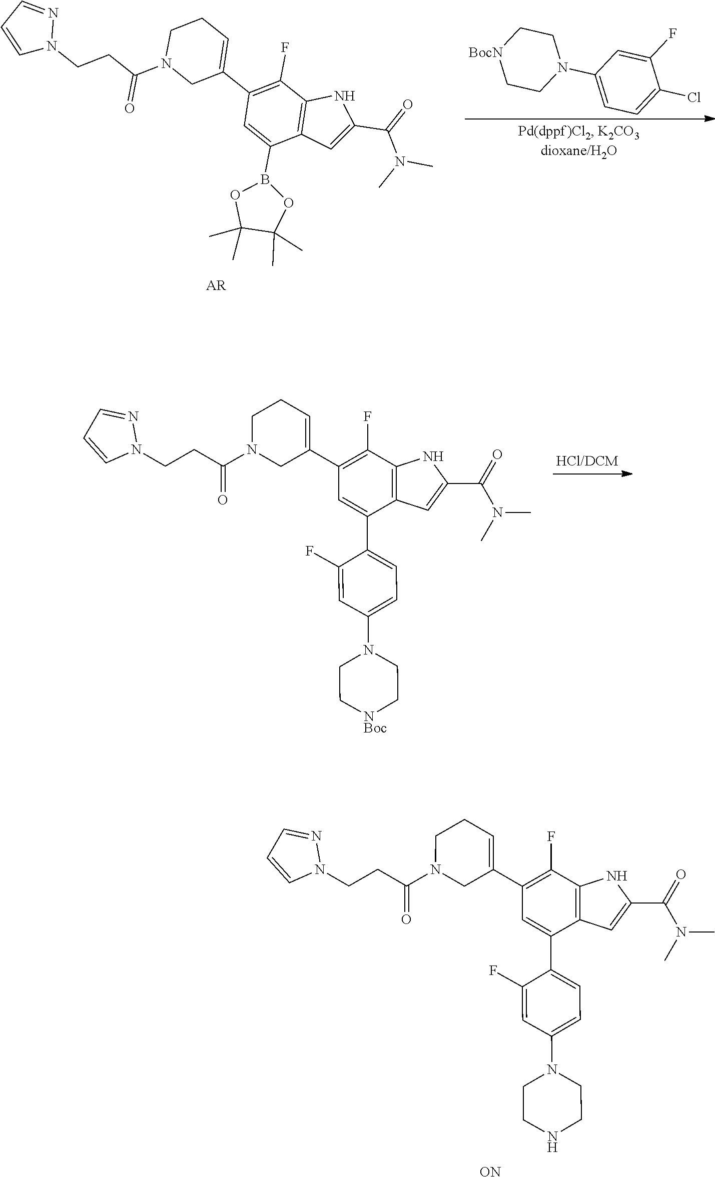

### Step 1—Tert-butyl 4-(4-(6-(1-(3-(1H-pyrazol-1-yl)propanoyl)-1,2,5,6-tetrahydropyridin-3-yl)-2-(dimethylcarbamoyl)-7-fluoro-1H-indol-4-yl)-3-fluorophenyl)piperazine-1-carboxylate

A mixture of tert-butyl 4-(4-chloro-3-fluorophenyl)piperazine-1-carboxylate (500 mg, 1.59 mmol, synthesized via Step 1 of Intermediate EN), 6-(1-(3-(1H-pyrazol-1-yl)propanoyl)-1,2,5,6-tetrahydropyridin-3-yl)-7-fluoro-N,N-dimethyl-4-(4,4,5,5-tetramethyl-1,3,2-dioxaborolan-2-yl)-1H-indole-2-carboxamide (936 mg, 1.75 mmol, Intermediate AR), XPhos Pd G3 (134 mg, 159 μmol), and K₃PO₄ (1.01 g, 4.77 mmol) in dioxane (5 mL) and H₂O (0.5 mL) was degassed and purged with N₂ three times. Then the mixture was stirred at 80° C. for 2 hrs under N₂ atmosphere. On completion, the reaction mixture was quenched with water (30 mL) and extracted with EA (3×30 mL). The combined organic layers were washed with aqueous NaCl (30 mL) and dried over anhydrous sodium sulfate, filtered and the filtrate was concentrated in vacuo to give the crude residue. The crude product was triturated with PE:EA=1:1 at 25° C. for 10 mins, then the mixture was filtered and the filter cake was dried in vacuo to give the title compound (850 mg, 78% yield) as a gray solid. LC-MS (ESI⁺) m/z 688.3 (M+H)⁺.

### Step 2—6-(1-(3-(1H-pyrazol-1-yl)propanoyl)-1,2,5,6-tetrahydropyridin-3-yl)-7-fluoro-4-(2-fluoro-4-(piperazin-1-yl)phenyl)-N,N-dimethyl-1H-indole-2-carboxamide

To a solution of tert-butyl 4-(4-(6-(1-(3-(1H-pyrazol-1-yl)propanoyl)-1,2,5,6-tetrahydropyridin-3-yl)-2-(dimethylcarbamoyl)-7-fluoro-1H-indol-4-yl)-3-fluorophenyl)piperazine-1-carboxylate (800 mg, 1.16 mmol) in DCM (10 mL) was added HCl/dioxane (2 M, 582 L). The mixture was then stirred at 25° C. for 1 hr. On completion, the reaction mixture was concentrated under reduced pressure to remove solvent to give the title compound (1 g, HCl) as a black solid. LC-MS (ESI⁺) m/z 588.2 (M+H)⁺.

### N-methylcyclopropanamine (CAS #5163-20-2) (Intermediate OO)

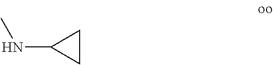

### 3-(3-fluoro-4-(4-(4-(4,4,5,5-tetramethyl-1,3,2-dioxaborolan-2-yl)phenyl)piperidin-1-yl)phenyl)piperidine-2,6-dione (Intermediate OP)

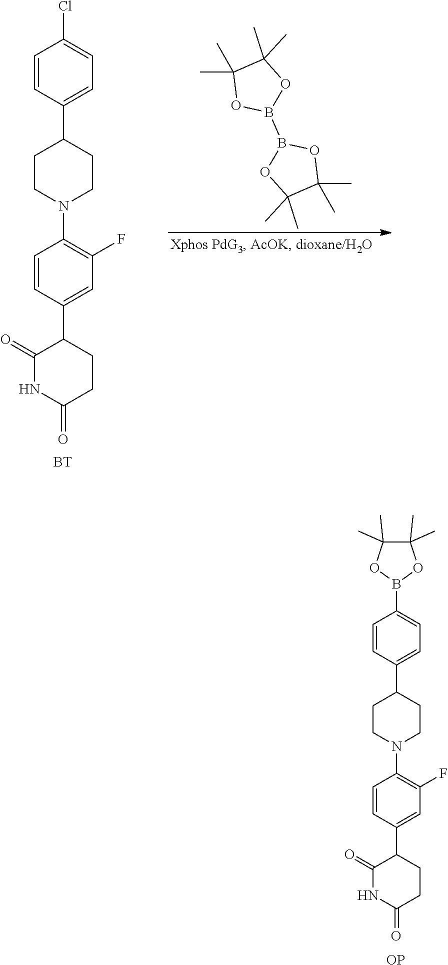

To a solution of 3-[4-[4-(4-chlorophenyl)-1-piperidyl]-3-fluoro-phenyl]piperidine-2,6-dione (1 g, 3 mmol, Intermediate BT) and 4,4,5,5-tetramethyl-2-(4,4,5,5-tetramethyl-1,3,2-dioxaborolan-2-yl)-1,3,2-dioxaborolane (950 mg, 3.74 mmol) in dioxane (10 mL) was added XPhos Pd G3 (211 mg, 249 μmol) and AcOK (734 mg, 7.48 mmol) at 25° C. Then the reaction was stirred at 80° C. for 1 hr. On completion, the reaction mixture was filtered and the filtrate was concentrated in vacuo to give the crude residue. The residue was purified by column chromatography (SiO₂, Petroleum ether:Ethyl acetate=1/1 to 0/1) to the title compound (1.05 g, 85% yield) as a gray solid. ¹H NMR (400 MHz, DMSO-d6) δ=10.9-10.8 (m, 1H), 7.63 (br d, J=8.0 Hz, 2H), 7.32 (br d, J=8.0 Hz, 2H), 7.08-6.99 (m, 2H), 6.99-6.93 (m, 1H), 3.81 (br dd, J=4.8, 11.6 Hz, 1H), 3.45 (br d, J=11.6 Hz, 2H), 2.78 (br t, J=10.0 Hz, 2H), 2.73-2.58 (m, 3H), 2.27-2.14 (m, 1H), 2.01 (td, J=4.4, 8.8 Hz, 1H), 1.89-1.76 (m, 4H), 1.29 (s, 12H).

### 6-(1-(3-(1H-1,2,3-triazol-1-yl)propanoyl)-1,2,5,6-tetrahydropyridin-3-yl)-4-(4-(1-(4-(2,6-dioxopiperidin-3-yl)-2-fluorophenyl)piperidin-4-yl)phenyl)-7-fluoro-H-indole-2-carboxylic acid (Intermediate OQ)

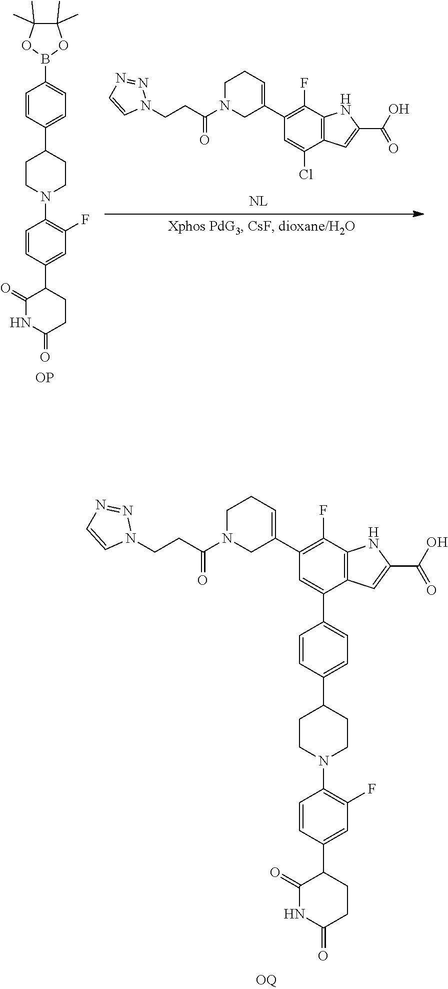

To a solution of 4-chloro-7-fluoro-6-[1-[3-(triazol-1-yl)propanoyl]-3,6-dihydro-2H-pyridin-5-yl]-1H-indole-2-carboxylic acid (50 mg, 120 μmol, Intermediate NL), 3-[3-fluoro-4-[4-[4-(4,4,5,5-tetramethyl-1,3,2-dioxaborolan-2-yl)phenyl]-1-piperidyl]phenyl]piperidine-2,6-dione (64.8 mg, 132 μmol, Intermediate OP) in dioxane (1 mL) was added XPhos Pd G3 (10.1 mg, 12.0 μmol) and CsF (54.5 mg, 359 μmol) in H₂O (0.2 mL) at 25° C. Then the reaction was stirred at 80° C. for 6 hrs under nitrogen atmosphere. On completion, the reaction mixture was filtered and the filtrate was concentrated in vacuo to give the crude residue. The residue was purified by column chromatography prep-TLC (SiO₂, THF: DCM=1/2) to the title compound (30 mg, 34% yield) as a white solid. LC-MS (ESI⁺) m/z 748.3 (M+H)⁺.

### Tert-butyl 4-(4-bromo-2,3-difluorophenyl)-3,3-difluoropiperidine-1-carboxylate (Intermediate OR)

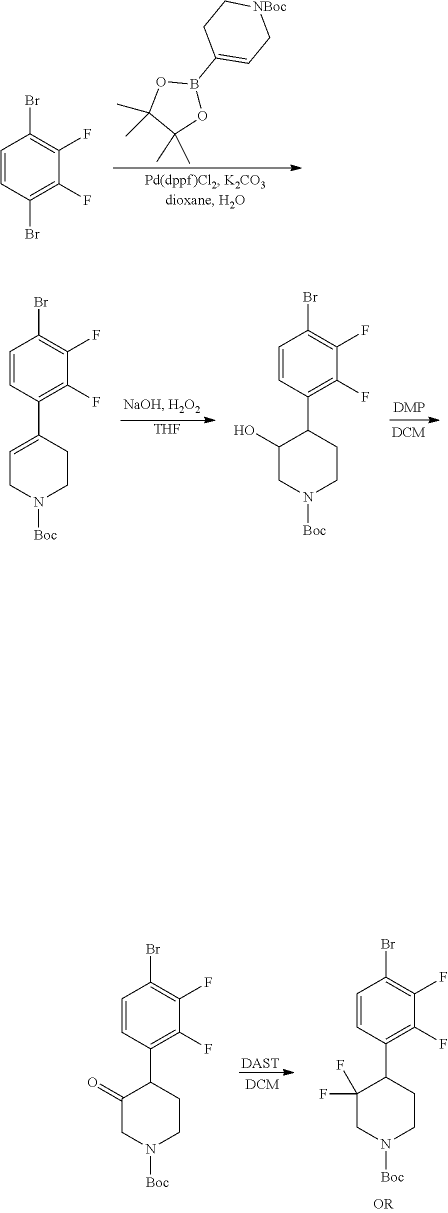

### Step 1—Tert-butyl 4-(4-bromo-2,3-difluorophenyl)-3,6-dihydropyridine-1 (2H)-carboxylate

A mixture of 1,4-dibromo-2,3-difluoro-benzene (16.4 g, 60.3 mmol, CAS #156682-52-9), tert-butyl 4-(4,4,5,5-tetramethyl-1,3,2-dioxaborolan-2-yl)-3,6-dihydro-2H-pyridine-1-carboxylate (16.7 g, 54.2 mmol, CAS #286961-14-6), K₂CO₃ (24.9 g, 180 mmol), and Pd(dppf)Cl₂ CH₂Cl₂ (4.92 g, 6.02 mmol) in dioxane (180 mL) and H₂O (45 mL) was degassed and purged with N₂ three times. Then the mixture was stirred at 80° C. for 12 hrs under N₂ atmosphere. On completion, the reaction mixture was concentrated in vacuo to give the crude residue. The residue was purified by column chromatography (SiO₂, Petroleum ether/Ethyl acetate=50/1 to 20/1) to give the title compound (9.5 g, 38% yield) as a white solid. LC-MS (ESI⁺) m/z 361.0 (M+H)⁺; ¹H NMR (400 MHz. DMSO-d₆) δ=7.59-7.46 (m, 1H), 7.25-7.13 (m, 1H), 6.09 (br s, 1H), 4.00 (br s, 2H), 3.53 (br t, J=5.2 Hz, 2H), 2.42 (br s, 2H), 1.43 (s, 9H).

### Step 2—Tert-butyl 4-(4-bromo-2,3-difluorophenyl)-3-hydroxypiperidine-1-carboxylate

To a solution of tert-butyl 4-(4-bromo-2,3-difluoro-phenyl)-3,6-dihydro-2H-pyridine-1-carboxylate (8.5 g, 23 mmol) in THF (90 mL) was added dropwise BH₃-THF (1 M, 56.7 mL) at 0° C. under N₂ atmosphere. The mixture was stirred at 25° C. for 12 hrs, and then NaOH (2.27 g. 56.7 mmol) in H₂O (10 mL) was added dropwise at 0° C. The mixture was stirred at this temperature for 0.5 hrs. Next, H₂O₂ (6.44 g, 56.7 mmol, 5.46 mL, 30% solution) was added dropwise. The resulting mixture was stirred at 50° C. for 2 hrs under N₂ atmosphere. On completion, the reaction mixture was quenched by addition of Na₂S₂O₃ (70 mL), diluted by water (90 mL) and extracted by ethyl acetate (3×200 mL). The combined organic layers were dried over anhydrous sodium sulfate, filtered and the filtrate was concentrated in vacuo to give the crude residue. The residue was purified by column chromatography (SiO₂. Petroleum ether/Ethyl acetate=6/1 to 3/1) to give the title compound (7.5 g. 50% yield) as a colorless oil. LC-MS (ESI⁺) n/z 413.8 (M+Na)⁺; ¹H NMR (400 MHz, DMSO-d₆) δ=7.52-7.37 (m, 1H), 7.21 (br t, J=6.8 Hz, 1H), 4.25-3.75 (m, 3H), 3.23-3.03 (m, 1H), 2.89-2.73 (m, 1H), 2.03-1.94 (m, 1H), 1.59 (br d, J=12.8 Hz, 1H), 1.42 (br s, 11H).

### Step 3—Tert-butyl 4-(4-bromo-2,3-difluorophenyl)-3-oxopiperidine-1-carboxylate

To a solution of tert-butyl 4-(4-bromo-2,3-difluoro-phenyl)-3-hydroxy-piperidine-1-carboxylate (13 g, 33 mmol) in DCM (150 mL) was added DMP (21.1 g, 49.7 mmol). The mixture was stirred at 25° C. for 4 hrs. On completion, the reaction mixture was added Na₂SO₃ (50 mL), then diluted by water (50 mL) and extracted by dichloromethane (3×200 mL). The combined organic layers were washed with NaHCO₃ (3×200 mL), dried over anhydrous sodium sulfate, filtered and the filtrate was concentrated in vacuo to give the crude residue. The residue was purified by column chromatography (SiO₂, Petroleum ether/Ethyl acetate=10/1 to 6/1) to give the title compound (5 g, 35% yield) as a colorless oil. LC-MS (ESI⁺) m/z 413.8 (M+H)⁺.

### Step 4—Tert-butyl 4-(4-bromo-2,3-difluorophenyl)-3,3-difluoropiperidine-1-carboxylate

A mixture of tert-butyl 4-(4-bromo-2,3-difluoro-phenyl)-3-oxo-piperidine-1-carboxylate (5 g. 13 mmol), DAST (4.13 g, 25.6 mmol, 3.39 mL) in DCM (50 mL) was degassed and purged with N₂ three times. Then the mixture was stirred at 25° C. for 4 hrs under N₂ atmosphere. On completion, the reaction mixture was added NaHCO₃ until pH was 6, then diluted with water (30 mL) and extracted with dichloromethane (3×90 mL). The combined organic layers were dried over anhydrous sodium sulfate, filtered and the filtrate was concentrated in vacuo to give the crude residue. The residue was purified by column chromatography (SiO₂, Petroleum ether/Ethyl acetate=20/1 to 10/1) to give the title compound (3 g, 50% yield) as a white solid. LC-MS (ESI⁺) m/z 357.8 (M+H)⁺; ¹H NMR (400 MHz. DMSO-d₆) δ=7.61-7.51 (m, 1H), 7.27 (br t, J=7.6 Hz, 1H), 4.30-4.03 (m, 2H), 3.84-3.63 (m, 1H), 3.58-3.34 (m, 1H), 3.13-2.92 (m, 1H), 2.15-1.97 (m, 1H), 1.85 (br d, J=13.2 Hz, 1H), 1.43 (s, 9H).

### 3-(4-((S)-4-(4-bromo-2,3-difluorophenyl)-3,3-difluoro-[1,4′-bipiperidin]-1′-yl)-3-fluorophenyl)piperidine-2,6-dione (Intermediate OS)

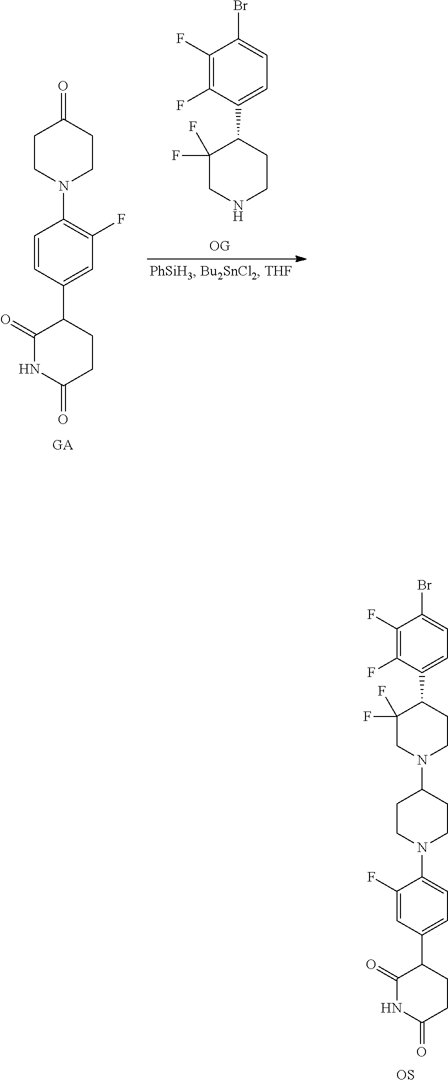

To a solution of (4S)-4-(4-bromo-2,3-difluoro-phenyl)-3,3-difluoro-piperidine (350 mg, 1.12 mmol, Intermediate OG) and 3-[3-fluoro-4-(4-oxo-1-piperidyl)phenyl]piperidine-2,6-dione (410 mg, 1.35 mmol. Intermediate GA) in THE (5 mL) was added phenylsilane (364 mg, 3.36 mmol) and dibutyl(dichloro)stannane (511 mg, 1.68 mmol). The mixture was then stirred at 60° C. for 6 hrs. On completion, the reaction mixture was filtered and washed with EA and DCM/MeOH. The filtrate was then concentrated under reduced pressure to give the title compound (270 mg) as a white solid. LC-MS (ESI⁺) m/z 599.9 (M+H)⁺; ¹H NMR (400 MHz, DMSO-d₆) δ=10.83 (s, 1H), 7.64 (br t, J=7.2 Hz, 1H), 7.23-6.93 (m, 4H), 4.14-3.88 (m, 2H), 3.84-3.78 (m, 1H), 3.67-3.57 (m, 1H), 3.55-3.42 (m, 8H), 2.78-2.63 (m, 3H), 2.21 (m, 3H), 2.05-1.87 (m, 3H).

### Tert-butyl (S)-4-(4-bromo-2,3-difluorophenyl)-3,3-difluoro-[1,4′-bipiperidine]-1′-carboxylate (Intermediate OT) and tert-butyl (R)-4-(4-bromo-2,3-difluorophenyl)-3,3-difluoro-[1,4′-bipiperidine]-1′-carboxylate (Intermediate OU)

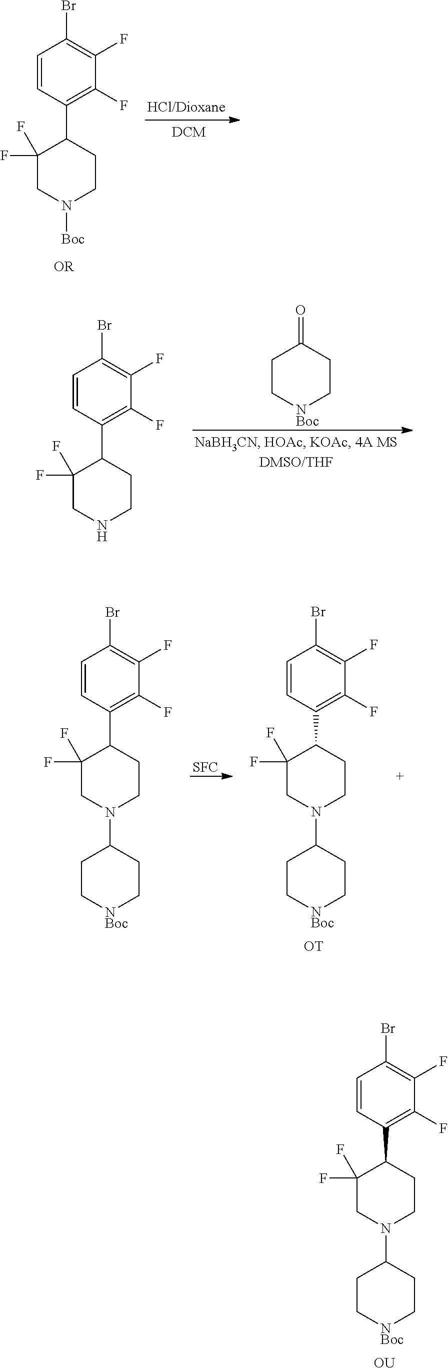

### Step 1—4-(4-Bromo-2,3-difluorophenyl)-3,3-difluoropiperidine compound with methane

To a solution of tert-butyl 4-(4-bromo-2,3-difluoro-phenyl)-3,3-difluoro-piperidine-1-carboxylate (1.5 g, 3.6 mmol, Intermediate OR) in DCM (2 mL) was added HCl/dioxane (2 M. 10.0 mL). The mixture was then stirred at 25° C. for 2 hrs. On completion, the reaction mixture was concentrated in vacuo to give the compound (1 g) as a white solid. LC-MS (ESI⁺) m/z 311.8 (M+H)⁺.

### Step 2—Tert-butyl 4-(4-bromo-2,3-difluorophenyl)-3,3-difluoro-[1,4′-bipiperidine]-1′-carboxylate

To a solution of 4-(4-bromo-2,3-difluoro-phenyl)-3,3-difluoro-piperidine (1 g, 3 mmol, HCl) in THF (5 mL), DMSO (5 mL) was added 4 Å molecular sieves (1.5 g, 2.87 mmol) and KOAc (844 mg, 8.61 mmol). After 10 min, tert-butyl 4-oxopiperidine-1-carboxylate (800 mg, 4.02 mmol, CAS #7 9099-07-3) and HOAc (516 mg, 8.61 mmol) was added. The mixture was stirred at 40° C. for 12 hrs. Then NaBH₃CN (360 mg, 5.74 mmol) was added at 0° C., and the final mixture was stirred at 40° C. for 2 hrs under N₂ atmosphere. On completion, the reaction mixture was filtered to give a filtrate. The filtrate was purified by reversed-phase HPLC (0.8 g/L ammonium bicarbonate) to give the title compound (600 mg, 40% yield) as a white solid. LC-MS (ESI⁺) m/z 495.0 (M+H)⁺.

### Step 3—Tert-butyl (S)-4-(4-bromo-2,3-difluorophenyl)-3,3-difluoro-[1,4′-bipiperidine]-1′-carboxylate and tert-butyl (R)-4-(4-bromo-2,3-difluorophenyl)-3,3-difluoro-[1,4′-bipiperidine]-1′-carboxylate

A solution of tert-butyl 4-(4-bromo-2,3-difluorophenyl)-3,3-difluoro-[1,4′-bipiperidine]-1′-carboxylate (900 mg) in EtOH (3 mL) was purified by SFC (column: DAICEL CHIRALPAK IK (250 mm×30 mm, 10 um); mobile phase: [CO₂-i-PrOH]; B %:25%, isocratic elution mode to give Tert-butyl (S)-4-(4-bromo-2,3-difluorophenyl)-3,3-difluoro-[1,4′-bipiperidine]-1′-carboxylate (360 mg), and tert-butyl (R)-4-(4-bromo-2,3-difluorophenyl)-3,3-difluoro-[1,4′-bipiperidine]-1′-carboxylate (360 mg) as a gray solids. LC-MS (ESI⁺) m/z 495.1 (M+H)⁺ for both isomers. The absolute stereochemistry of the enantiomers was assigned arbitrarily.

### (S)-4-(4-bromo-2,3-difluorophenyl)-3,3-difluoro-14′-bipiperidine (Intermediate OV)

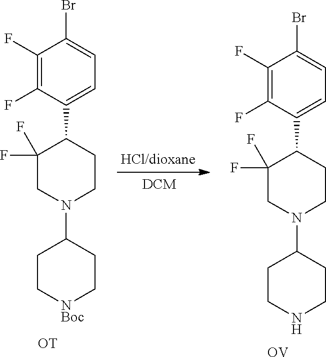 [SMILES: CC(C)(C)OC(=O)N1CCC(N2CC[C@@H](c3ccc(Br)c(F)c3F)C(F)(F)C2)CC1 ; Fc1c(Br)ccc([C@@H]2CCN(C3CCNCC3)CC2(F)F)c1F ; "OT" ; "OV"]

To a solution of tert-butyl 4-[(4S)-4-(4-bromo-2,3-difluoro-phenyl)-3,3-difluoro-1-piperidyl]piperidine-1-carboxylate (120 mg, 242 μmol, Intermediate OT) in DCM (4 mL) was added HCl/dioxane (4 M, 0.5 mL). The mixture was then stirred at 25° C. for 2 hrs. On completion, the reaction mixture was concentrated in vacuo to give a residue. The crude residue was purified by reversed-phase HPLC (0.8 g/L ammonium bicarbonate) to give the compound (80 mg, 87% yield) as a white solid. LC-MS (ESI⁺) m/z 395.0 (M+H)⁺.

### 3-(6-((S)-4-(4-bromo-2,3-difluorophenyl)-3,3-difluoro-[1,4′-bipiperidin]-1′-yl)-5-fluoropyridin-3-yl)piperidine-2,6-dione (Intermediate OW)

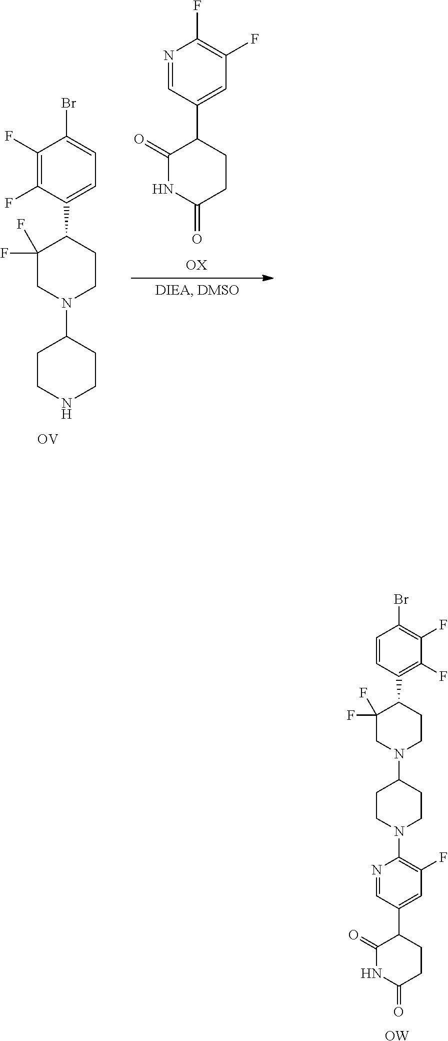

To a solution of (4S)-4-(4-bromo-2,3-difluoro-phenyl)-3,3-difluoro-1-(4-piperidyl)piperidine (70 mg, 177 μmol, Intermediate OV), 3-(5,6-difluoro-3-pyridyl)piperidine-2,6-dione (52.0 mg, 230 μmol, Intermediate OX) in DMSO (2 mL) was added DIEA (228 mg, 1.77 mmol). The mixture was then stirred at 100° C. for 12 hrs. On completion, the reaction mixture was quenched with water (10 mL) and extracted with ethyl acetate (3×15 mL). The combined organic layers were washed with brine (30 mL), dried over anhydrous sodium sulfate, filtered and the filtrate was concentrated in vacuo to give the crude residue. The residue was purified by column chromatography (SiO₂, Petroleum ether/Ethyl acetate=3/1 to 0/1) to give the title compound (40 mg, 32% yield) as a brown solid. LC-MS (ESI⁺) m/z 603.1 (M+H)⁺.

### 3-(5,6-Difluoro-3-pyridyl)piperidine-2,6-dione (Intermediate OX)

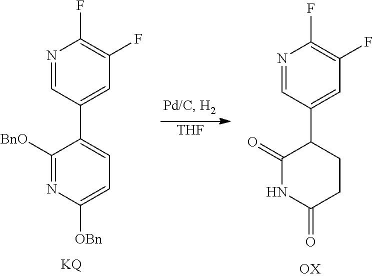

To a solution of 2,6-dibenzyloxy-3-(5,6-difluoro-3-pyridyl)pyridine (2.3 g, 5.7 mmol, Intermediate KQ) in THF (20 mL) was added Pd/C (2.3 g, 2.2 mmol. 10 wt %) under N₂ atmosphere. The suspension was degassed and purged with H₂ three times. The mixture was then stirred under H₂ (40 Psi.) at 30° C. for 48 h. On completion, the reaction was filtered through kieselguhr very carefully, and the filtrated was concentrated in vacuo to give a residue. The residue was purified by column chromatography (SiO₂, Petroleum ether/Ethyl acetate=5/1 to 2/1 to DCM:THF=4:1) to give the compound (1.06 g, 83% yield) as an off-white solid. LC-MS (ESI⁺) m/z 227.2 (M+H)⁺.

### (R)-4-(4-bromo-2,3-difluorophenyl)-3,3-difluoro-1,4′-bipiperidine (Intermediate OY)

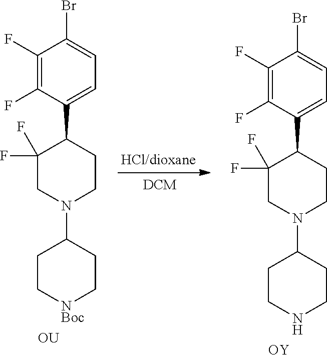 [SMILES: CC(C)(C)OC(=O)N1CCC(N2CC[C@H](c3ccc(Br)c(F)c3F)C(F)(F)C2)CC1 ; Fc1c(Br)ccc([C@H]2CCN(C3CCNCC3)CC2(F)F)c1F ; "OU" ; "OY"]

To a solution of tert-butyl 4-[(4R)-4-(4-bromo-2,3-difluoro-phenyl)-3,3-difluoro-1-piperidyl]piperidine-1-carboxylate (120 mg, 242 μmol, Intermediate OU) in DCM (4 mL) was added HCl/dioxane (4 M, 0.5 mL). The mixture was then stirred at 25° C. for 2 hrs. On completion, the reaction mixture was concentrated in vacuo to give a residue. The crude residue was purified by reversed-phase HPLC (0.8 g/L ammonium bicarbonate) to give the compound (80 mg, 87% yield) as a white solid. LC-MS (ESI⁺) m/z 395.0 (M+H)⁺.

### 3-(6-((R)-4-(4-bromo-2,3-difluorophenyl)-3,3-difluoro-[1,4′-bipiperidin]-1′-yl)-5-fluoropyridin-3-yl)piperidine-2,6-dione (Intermediate OZ)

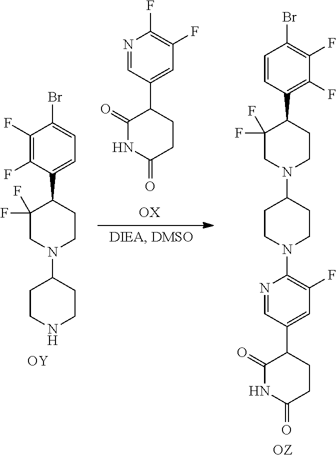

To a solution of (4R)-4-(4-bromo-2,3-difluoro-phenyl)-3,3-difluoro-1-(4-piperidyl)piperidine (70 mg, 177 μmol, Intermediate OY) and 3-(5,6-difluoro-3-pyridyl)piperidine-2,6-dione (52.0 mg, 230 μmol, Intermediate OX) in DMSO (2 mL) was added DIEA (228 mg, 1.77 mmol). The mixture was the stirred at 100° C. for 12 hrs. On completion, the reaction mixture was quenched with water (10 mL) and extracted with ethyl acetate (3×15 mL). The combined organic layers were washed with brine (30 mL), dried over anhydrous sodium sulfate, filtered and the filtrate was concentrated in vacuo to give the crude residue. The residue was purified by column chromatography (SiO₂, Petroleum ether/Ethyl acetate=3/1 to 0/1) to give the title compound (40 mg, 32% yield) as a brown solid. LC-MS (ESI⁺) m/z 603.1 (M+H)⁺.

### 4-(4-Chloro-2,3-difluoro-phenyl)-1-(4-piperidyl)piperidine (Intermediate PA)

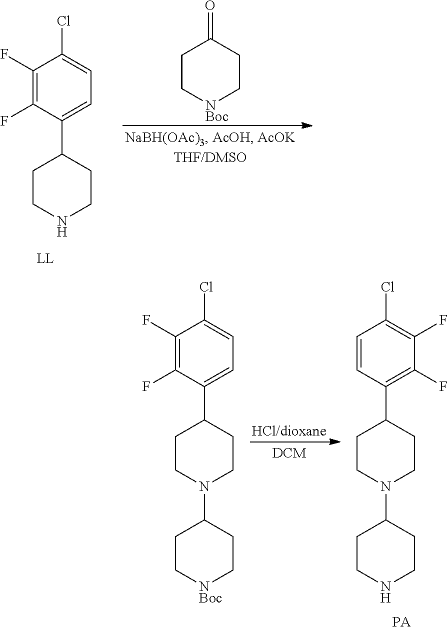 [SMILES: Fc1c(Cl)ccc(C2CCNCC2)c1F ; CC(C)(C)OC(=O)N1CCC(=O)CC1 ; CC(C)(C)OC(=O)N1CCC(N2CCC(c3ccc(Cl)c(F)c3F)CC2)CC1 ; Fc1c(Cl)ccc(C2CCN(C3CCNCC3)CC2)c1F]

### Step 1—Tert-butyl 4-(4-chloro-2,3-difluorophenyl)-[1,4′-bipiperidine]-1′-carboxylate

To a solution of 4-(4-chloro-2,3-difluoro-phenyl)piperidine (500 mg, 1.86 mmol, HCl, Intermediate LL) in THF (2 mL) and DMSO (2 mL) was added KOAc (458 mg, 4.66 mmol), tert-butyl 4-oxopiperidine-1-carboxylate (483 mg, 2.42 mmol, CAS #79099-07-3), HOAc (280 mg, 4.66 mmol, 267 L), and NaBH₃CN (352 mg, 5.59 mmol). The mixture was then stirred at 25° C. for 24 hrs. On completion, the reaction mixture was quenched with water (30 mL) and extracted with ethyl acetate (3×30 mL). The combined organic layers were washed with aqueous NaCl (20 mL) and dried over anhydrous sodium sulfate, filtered and the filtrate was concentrated in vacuo to give the crude residue. The crude residue was purified by reversed-phase HPLC (0.1% FA condition) to give the title compound (250 mg, 32% yield) as a white oil. LC-MS (ESI⁺) m/z 415.2 (M+H)⁺.

### Step 2—4-(4-Chloro-2,3-difluoro-phenyl)-1-(4-piperidyl)piperidine

To a solution of tert-butyl 4-[4-(4-chloro-2,3-difluoro-phenyl)-1-piperidyl]piperidine-1-carboxylate (600 mg, 1.45 mmol) in DCM (6 mL) was added HCl/dioxane (2 M, 6 mL). The mixture was then stirred at 25° C. for 2 h. On completion, the mixture was filtered and the filter cake was dried under reduced pressure to give the title compound (450 mg, HCl salt) as a white solid. LC-MS (ESI⁺) m/z 315.1M+H)⁺.

### 3-(6-(4-(4-Chloro-2,3-difluorophenyl)-[1,4′-bipiperidin]-1′-yl)-5-fluoropyridin-3-yl)piperidine-2,6-dione (Intermediate PB)

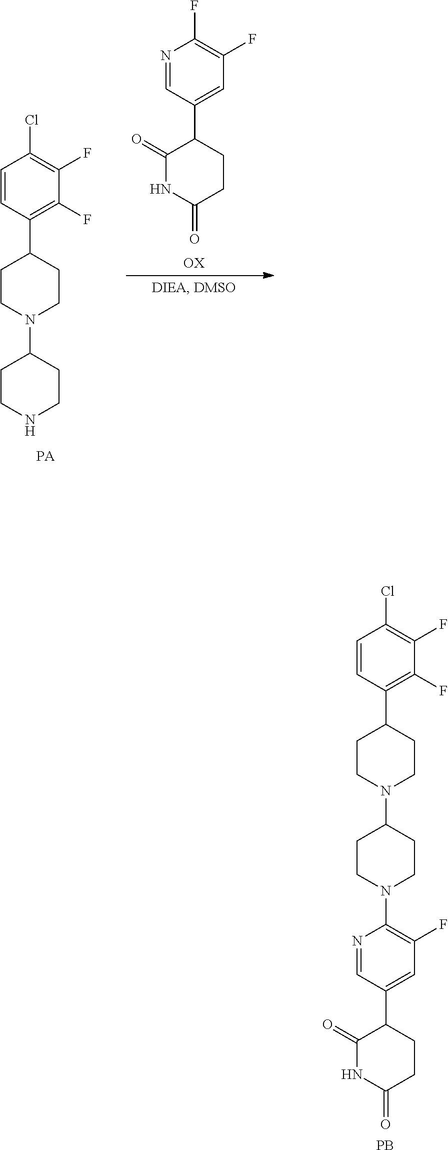

To a solution of 4-(4-chloro-2,3-difluoro-phenyl)-1-(4-piperidyl)piperidine (300 mg. 952 μmol, Intermediate PA), 3-(5,6-difluoro-3-pyridyl)piperidine-2,6-dione (647 mg, 2.86 mmol, Intermediate OX) in DMSO (4 mL) was added DIEA (370 mg, 2.86 mmol). The mixture was then stirred at 100° C. for 12 hrs. Upon completion, the mixture was filtered and concentrated under reduced pressure to give a residue. The residue was purified by prep-HPLC (FA condition) to give the title compound (300 mg, 44% yield, FA) as a white solid. LC-MS (ESI⁺) m/z 521.1M+H)⁺.

### 4-(4-Chlorophenyl)-3,3-difluoropiperidine (Intermediate PC)

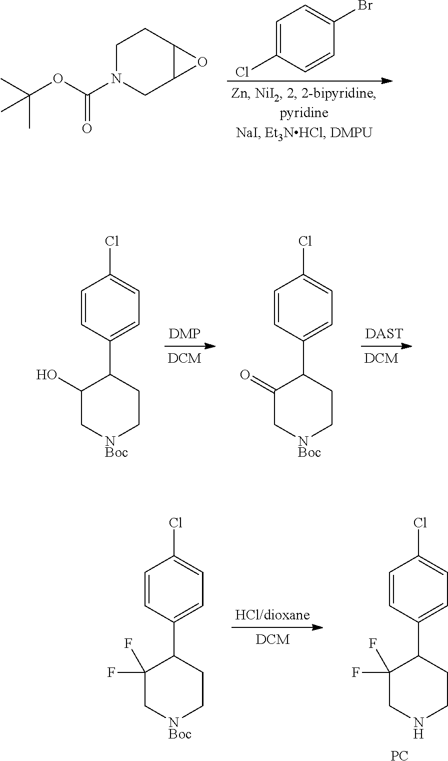

### Step 1—Tert-butyl 4-(4-chlorophenyl)-3-hydroxypiperidine-1-carboxylate

A mixture of 1-bromo-4-chlorobenzene (10 g, 52 mmol. CAS #161157-50-2), tert-butyl 7-oxa-3-azabicyclo[4.1.0]heptane-3-carboxylate (10.4 g, 52.2 mmol, CAS #3106-39-8), NaI (1.96 g, 13.1 mmol), N,N-diethylethanamine hydrochloride (7.19 g, 52.2 mmol). 2-(2-pyridyl)pyridine (815.76 mg, 5.2 mmol), pyridine (826 mg, 10.5 mmol), diiodonickel (1.63 g, 5.22 mmol) and Zn (6.83 g, 104.5 mmol) in DMPU (100 mL) was degassed and purged with N₂ three times. Then the mixture was stirred at 80° C. for 12 hrs under N₂ atmosphere. The mixture was diluted with water (50 mL) and extracted with ethyl acetate (50 mL×2), dried over sodium sulfate, filtered and concentrated under reduced pressure to give a residue. The residue was purified by prep-HPLC (FA condition) to give the title compound. (3.3 g, 14% yield) as a white solid. ¹H NMR (400 MHz, DMSO-d6) 5=7.39-7.35 (m, 2H), 7.33-7.29 (m, 2H), 4.28-3.93 (m, 3H), 3.87-3.43 (m, 2H), 1.72 (d, J=12.0 Hz, 1H), 1.64-1.54 (m, 1H), 1.47 (s, 10H).

### Step 2—Tert-butyl 4-(4-chlorophenyl)-3-oxopiperidine-1-carboxylate

To a solution of tert-butyl 4-(4-chlorophenyl)-3-hydroxypiperidine-1-carboxylate (3.3 g, 11 mmol) in DCM (40 mL) was added DMP (6.73 g. 15.9 mmol). The mixture was then stirred at 25° C. for 2 hrs. On completion, the mixture was diluted with water (50 mL) and extracted with ethyl acetate (50 mL×2), dried over sodium sulfate, filtered and concentrated under reduced pressure to give a residue. The residue was purified by silica gel column chromatography (SiO₂, PE:EA=50/1 to 40/1) to give the title compound (3 g, 64% yield) as a yellow oil. LC-MS (ESI⁺) m/z 331.9 (M+Na)⁺; ¹H NMR (400 MHz, CHLOROFORM-d) δ=7.25 (d, J=8.4 Hz, 2H), 7.02-6.98 (m, 2H), 4.26-4.11 (m, 2H), 4.05-3.85 (m, 2H), 3.60-3.51 (m, 1H), 3.50-3.38 (m, 2H), 1.42 (s, 9H).

### Step 3—Tert-butyl 4-(4-chlorophenyl)-3,3-difluoropiperidine-1-carboxylate

To a solution of tert-butyl 4-(4-chlorophenyl)-3-oxo-piperidine-1-carboxylate (3 g. 7 mmol) in DCM (30 mL) was added DAST (1.64 g. 10.2 mmol). The mixture was then stirred at 0-25° C. for 12 hrs. Upon completion, the mixture was diluted with water (50 mL) and extracted with ethyl acetate (50 mL×2), dried over sodium sulfate, filtered and concentrated under reduced pressure. The residue was purified by prep-HPLC (FA condition) to give the title compound (1.7 g, 73% yield) as a white solid. LC-MS (ESI⁺) m/z 275.9 (M−55)⁺: ¹H NMR (400 MHz, CDCl₃-d) δ=7.27-7.23 (m, 2H), 7.19-7.13 (m, 2H), 5.22 (s, 1H), 4.61-4.05 (m, 2H), 3.12-2.87 (m, 2H), 2.76 (s, 1H), 2.15-2.00 (m, 1H), 1.85-1.72 (m, 1H), 1.42 (s. 8H).

### Step 4—4-(4-Chlorophenyl)-3,3-difluoropiperidine

To a solution of tert-butyl 4-(4-chlorophenyl)-3,3-difluoropiperidine-1-carboxylate (1.7 g, 5.1 mmol) in DCM (10 mL) was added HCl/dioxane (2 M, 15 mL). The mixture was then stirred at 25° C. for 30 mins. Upon completion, the mixture was filtered and concentrated under reduced pressure to give a residue. The residue was purified by prep-HPLC (Neutral condition) to give the title compound (900 mg, 75% yield) as a white solid. LC-MS (ESI⁺) m/z 231.9 (M+Na)⁺.

### (S)-2,6-bis(benzyloxy)-3-(4-(4-(4-chlorophenyl)-3,3-difluoropiperidin-1-yl)-3-fluorophenyl)pyridine (Intermediate PD) & (R)-2,6-bis(benzyloxy)-3-(4-(4-(4-chlorophenyl)-3,3-difluoropiperidin-1-yl)-3-fluorophenyl)pyridine (Intermediate PE)

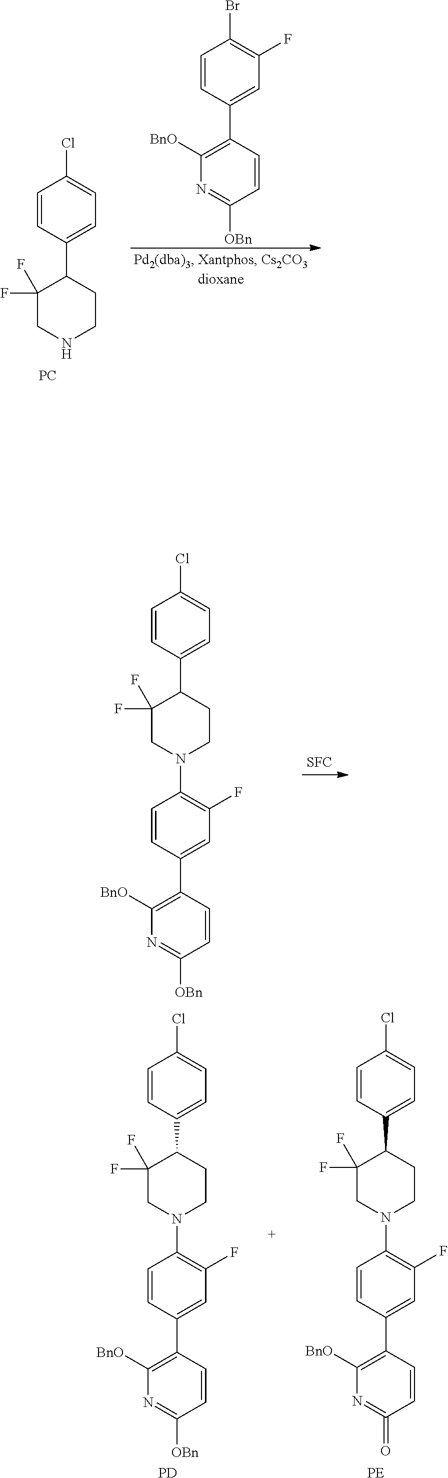

### Step 1—2,6-Bis(benzyloxy)-3-(4-(4-(4-chlorophenyl)-3,3-difluoropiperidin-1-yl)-3-fluorophenyl)pyridine

A mixture of 4-(4-chlorophenyl)-3,3-difluoropiperidine (824 mg, 3.56 mmol, Intermediate PC), 2,6-bis(benzyloxy)-3-(4-bromo-3-fluorophenyl)pyridine (1.98 g, 4.27 mmol), Pd₂(dba)₃ (326 mg, 356 μmol), Xantphos (206 mg, 356 μmol) and Cs₂CO₃ (2.9 g, 8.9 mmol) in dioxane (2 mL) was degassed and purged with N₂ three times. Then the mixture was stirred at 100° C. for 2 hrs under N₂ atmosphere. Upon completion, the mixture was filtered and concentrated under reduced pressure to give the residue. The residue was purified by prep-HPLC to give the title compound (1 g, 45% yield) as a white solid. LC-MS (ESI⁺) m/z 615.2 (M+H)⁺.

### Step 2—(S)-2,6-bis(benzyloxy)-3-(4-(4-(4-chlorophenyl)-3,3-difluoropiperidin-1-yl)-3-fluorophenyl)pyridine & (R)-2,6-bis(benzyloxy)-3-(4-(4-(4-chlorophenyl)-3,3-difluoropiperidin-1-yl)-3-fluorophenyl)pyridine

2,6-Bis(benzyloxy)-3-(4-(4-(4-chlorophenyl)-3,3-difluoropiperidin-1-yl)-3-fluorophenyl)pyridine (1 g, 1.61 mmol) was separated by SFC (column: DAICEL CHIRALPAK AD (250 mm*30 mm, 10 um); mobile phase: [CO2-i-PrOH (0.1% NH3H2O)]; B %:50%, isocratic elution mode) to give the first eluting peak (S)-2,6-bis(benzyloxy)-3-(4-(4-(4-chlorophenyl)-3,3-difluoropiperidin-1-yl)-3-fluorophenyl)pyridine (348 mg, 34% yield) as a white solid and the second eluting peak (R)-2,6-bis(benzyloxy)-3-(4-(4-(4-chlorophenyl)-3,3-difluoropiperidin-1-yl)-3-fluorophenyl)pyridine (415 mg, 39% yield) as a white solid. LC-MS (ESI⁺) m/z 615.1 (M+H)⁺ for both isomers. The absolute stereochemistry of the enantiomers was assigned arbitrarily.

### 3-(4-((S)-3,3-difluoro-4-(4-(4,4,5,5-tetramethyl-1,3,2-dioxaborolan-2-yl)phenyl)piperidin-1-yl)-3-fluorophenyl)piperidine-2,6-dione (Intermediate PF)

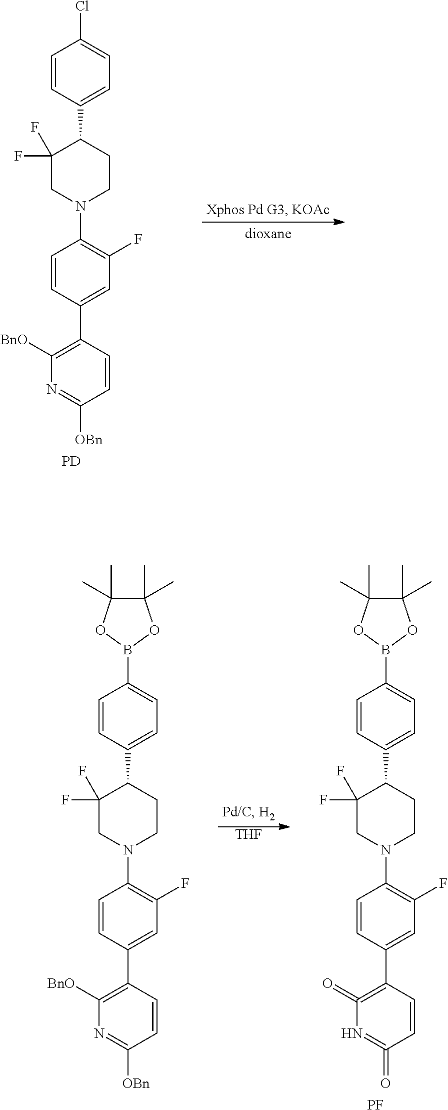

### Step 1—(S)-2,6-bis(benzyloxy-)-3-(4-(3,3-difluoro-4-(4-(4,4,5,5-tetramethyl-1,3,2-dioxaborolan-2-yl)phenyl)piperidin-1-yl)-3-fluorophenyl)pyridine

A mixture of(S)-2,6-bis(benzyloxy)-3-(4-(4-(4-chlorophenyl)-3,3-difluoropiperidin-1-yl)-3-fluorophenyl)pyridine (348 mg, 566 μmol, Intermediate PD), 4,4,4′,4′,5,5,5′,5′-octamethyl-2,2′-bi(1,3,2-dioxaborolane) (431 mg, 1.70 mmol), XPhos Pd G3 (47.9 mg, 56.6 μmol), and AcOK (166.6 mg, 1.70 mmol) in dioxane (10 mL) was degassed and purged with N₂ three times. Then the mixture was stirred at 80° C. for 1 hr under N₂ atmosphere. The mixture was diluted with water (15 mL) and extracted with ethyl acetate (15 mL×2), dried over sodium sulfate, filtered and concentrated under reduced pressure. The residue was purified by silica gel column chromatography (SiO₂, PE:EA=50/1 to 40/1) to give the title compound (400 mg. 90% yield) as a white solid. LC-MS (ESI⁺) m/z 707.3 (M+H)⁺.

### Step 2—3-(4-((S)-3,3-difluoro-4-(4-(4,4,5,5-tetramethyl-1,3,2-dioxaborolan-2-yl)phenyl)piperidin-1-yl)-3-fluorophenyl)piperidine-2,6-dione

A mixture of 2,6-dibenzyloxy-3-[4-[(4S)-3,3-difluoro-4-[4-(4,4,5,5-tetramethyl-1,3,2-dioxaborolan-2-yl)phenyl]-1-piperidyl]-3-fluoro-phenyl]pyridine (400 mg, 566 μmol), Pd/C (400 mg, 376 μmol, 10 wt %) in THF (10 mL) was degassed and purged with H₂ three times. Then the mixture was stirred at 30° C. for 12 hrs under H₂ atmosphere. Upon completion, the mixture was diluted with water (15 mL) and extracted with ethyl acetate (15 mL×2), dried over sodium sulfate, filtered and concentrated under reduced pressure to give the title compound (200 mg, 54% yield) as a white solid. LC-MS (ESI⁺) m/z 529.2 (M+H)⁺.

### 4-(4-Bromophenyl)-1-(3-chloro-2-fluoro-4-iodophenyl)piperidine (Intermediate PG)

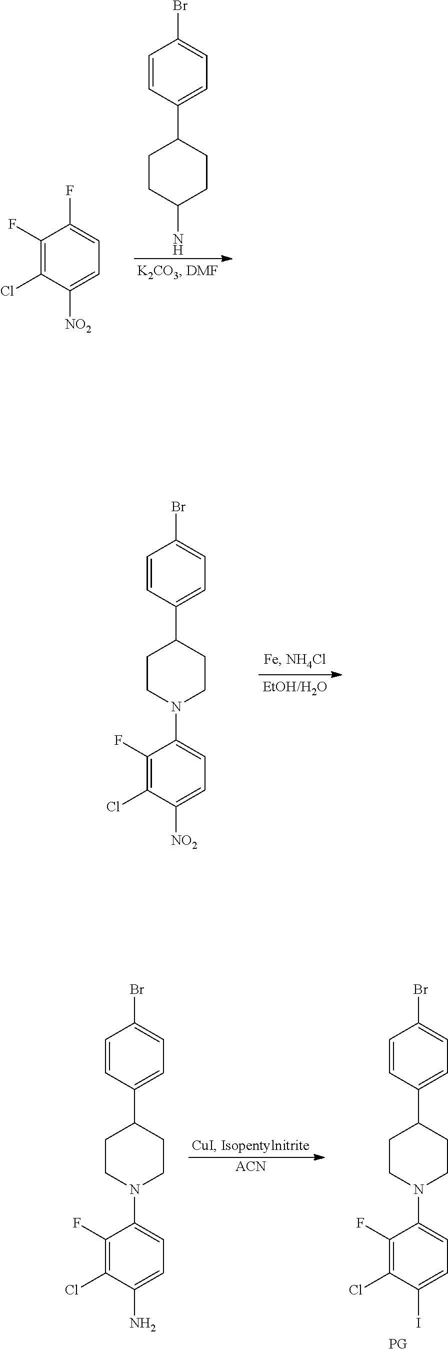

### Step 1—4-(4-Bromophenyl)-1-(3-chloro-2-fluoro-4-nitrophenyl)piperidine

To a solution of 2-chloro-3,4-difluoro-1-nitrobenzene (5 g, 25.8 mmol, CAS #169468-83-1) and 4-(4-bromophenyl)piperidine (6.20 g, 25.8 mmol, CAS #80980-89-8) in DMF (50 mL) was added K₂CO₃ (10.7 g, 77.5 mmol). The mixture was then stirred at 80° C. for 4 hr. On completion, the reaction mixture was quenched by water (100 mL) and filtered. The filtrate cake was dried to give the title compound (10 g) as yellow solid. LC-MS (ESI⁺) m/z 414.9 (M+H)⁺.

### Step 2—4-(4-(4-Bromophenyl)piperidin-1-yl)-2-chloro-3-fluoroaniline

To a solution of 4-(4-bromophenyl)-1-(3-chloro-2-fluoro-4-nitrophenyl)piperidine (10 g, 24 mmol) in EtOH (20 mL) and H₂O (10 mL) was added Fe (13.5 g, 241 mmol) and NH₄Cl (12.9 g, 241 mmol). The mixture was then stirred at 80° C. for 1 hr. On completion, the reaction mixture was filtered and the filtrate was concentrated in vacuo to give the title compound (8 g. 86% yield) as yellow solid. LC-MS (ESI⁺) m/z 385.0 (M+H)⁺.

### Step 3—4-(4-Bromophenyl)-1-(3-chloro-2-fluoro-4-iodophenyl)piperidine

To a solution of 4-[4-(4-bromophenyl)-1-piperidyl]-2-chloro-3-fluoro-aniline (4.4 g, 12 mmol) in ACN (50 mL) was added tert-butyl nitrite (1.77 g, 17.2 mmol, 2.05 mL) and CuI (2.62 g, 13.7 mmol). The mixture was then stirred at 50° C. for 12 hrs. On completion, the reaction mixture was concentrated under reduced pressure to remove solvent. The residue was purified by column chromatography (SiO₂, Petroleum ether/Ethyl acetate=10/1 to 1/1) to give the title compound (2 g, 35% yield) as a red solid. LC-MS (ESI+) m/z 494.5 (M+H)⁺.

### 3-(4-(4-(4-Bromophenyl)piperidin-1-yl)-2-chloro-3-fluorophenyl)piperidine-2,6-dione (Intermediate PH)

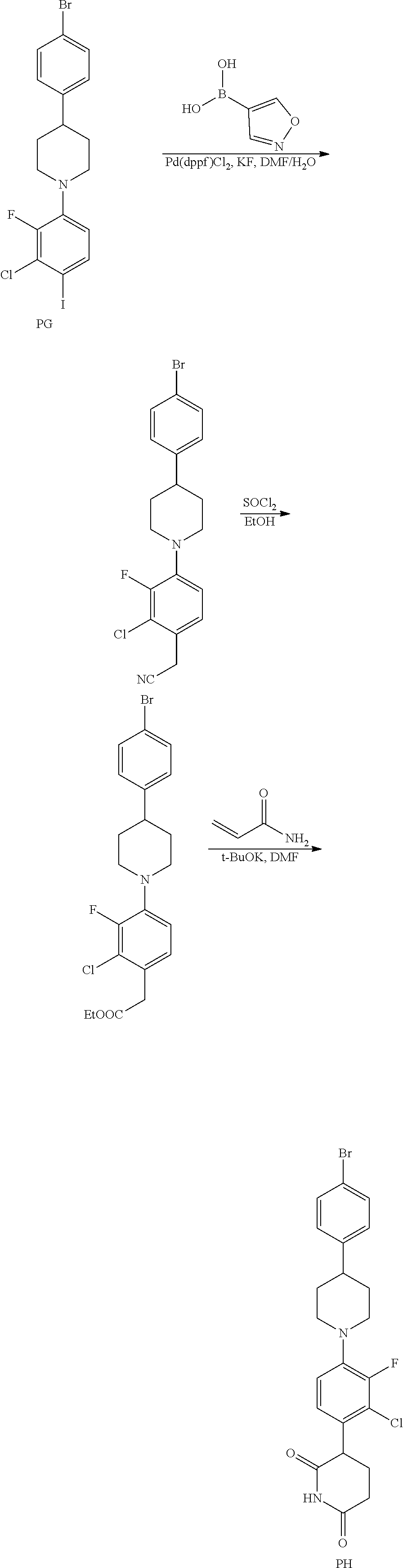

### Step 1—2-(4-(4-(4-Bromophenyl)piperidin-1-yl)-2-chloro-3-fluorophenyl)acetonitrile

To a solution of 4-(4-bromophenyl)-1-(3-chloro-2-fluoro-4-iodo-phenyl)piperidine (2 g, 4 mmol, Intermediate PG), isoxazol-4-ylboronic acid (548 mg, 4.85 mmol, CAS #1008139-25-0) in DMF (10 mL) was added Pd(dppf)Cl₂ (296 mg, 404 μmol), KF (705 mg, 12.1 mmol), and H₂O (219 mg, 12.1 mmol, 218 μL). The mixture was stirred at 80° C. for 4 hrs. On completion, the reaction mixture was quenched by addition of H₂O (200 mL) at 25° C., and then diluted with EA (500 mL) and extracted with EA (300 mL×2). The combined organic layers were washed with aqueous NaCl (400 mL×2), dried over Na₂SO₄, filtered and concentrated under reduced pressure to give a residue. The residue was purified by column chromatography (SiO₂, Petroleum ether/Ethyl acetate=10/1 to 1/1) to give the title compound (500 mg, 30% yield) as a white solid. LC-MS (ESI+) m/z 406.9 (M+H)⁺.

### Step 2—Methyl 2-(4-(4-(4-bromophenyl)piperidin-1-yl)-2-chloro-3-fluorophenyl)acetate

To a solution of 2-[4-[4-(4-bromophenyl)-1-piperidyl]-2-chloro-3-fluoro-phenyl]acetonitrile (440 mg, 1.08 mmol) in EtOH (4 mL) was added SOCl₂ (1.64 g, 13.8 mmol, 1 mL) at 0° C. The mixture was stirred at 25° C. for 16 hrs. On completion, the reaction mixture was quenched by addition of NaHCO₃ (aq) (10 mL) at 0 C, and then diluted with H₂O (20 mL) and extracted with EA (50 mL×2). The combined organic layers were dried over Na₂SO₄, filtered and concentrated under reduced pressure to give the title compound (400 mg) as a yellow solid. LC-MS (ESI⁺) m/z 455.0 (M+H)⁺.

### Step 3—3-(4-(4-(4-Bromophenyl)piperidin-1-yl)-2-chloro-3-fluorophenyl)piperidine-2,6-dione

To a solution of ethyl 2-(4-(4-(4-bromophenyl)piperidin-1-yl)-2-chloro-3-fluorophenyl)acetate (370 mg, 813 μmol) and prop-2-enamide (57.8 mg, 813 μmol, 56.1 VL, CAS #9003-05-8) in THF (10 mL) was added tBuOK (1 M, 894 μL) at 0° C. The mixture was the stirred at 40° C. for 12 hrs. Upon completion, the mixture was added HCl (1N) until the pH was 3, then extracted with dichloromethane (2×10 mL). The combined organic layers were dried over anhydrous sodium sulfate, filtered and the filtrate was concentrated in vacuo to give the title compound (100 mg, 26% yield) as yellow solid. LC-MS (ESI⁺) m/z 481.1 (M+H)⁺.

### 6-(1-(3-(1H-pyrazol-1-yl)propanoyl)-1,2,5,6-tetrahydropyridin-3-yl)-4-(4-bromo-2-chloro-5-fluorophenyl)-7-fluoro-N,N-dimethyl-1H-indole-2-carboxamide (Intermediate PI)

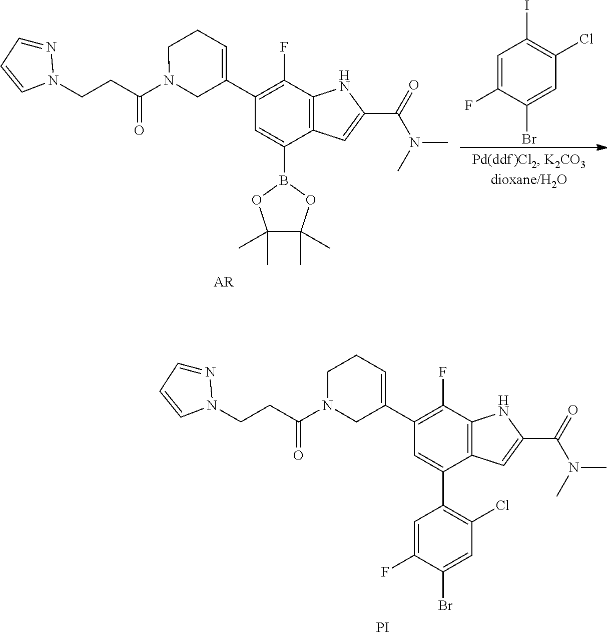

A mixture of 1-bromo-5-chloro-2-fluoro-4-iodobenzene (300 mg, 895 μmol, CAS #1000572-73-5), 6-(1-(3-(1H-pyrazol-1-yl)propanoyl)-1,2,5,6-tetrahydropyridin-3-yl)-7-fluoro-N,N-dimethyl-4-(4,4,5,5-tetramethyl-1,3,2-dioxaborolan-2-yl)-1H-indole-2-carboxamide (479 mg, 895 μmol, Intermediate AR), Pd(dppf)Cl₂ (65.5 mg, 89.5 μmol), and K₂CO₃ (371 mg, 2.68 mmol) in dioxane (2 mL) and H₂O (0.2 mL) was degassed and purged with N₂ three times. Then the mixture was stirred at 60° C. for 3 hrs under N₂ atmosphere. On completion, the reaction mixture was quenched with water (30 mL) and extracted with dichloromethane (3×30 mL). The combined organic layers were washed with aqueous NaCl (30 mL) and dried over anhydrous sodium sulfate, filtered and the filtrate was concentrated in vacuo to give the crude residue. The crude residue was purified by reversed-phase HPLC (0.1% FA condition) to give the title compound (500 mg, 91% yield) as a brown solid. LC-MS (ESI⁺) m/z 618.0 (M+H)⁻.

### 6-(1-(3-(1H-pyrazol-1-yl)propanoyl)-1,2,5,6-tetrahydropyridin-3-yl)-4-(2-chloro-5-fluoro-4-(piperidin-4-yl)phenyl)-7-fluoro-N,N-dimethyl-1H-indole-2-carboxamide (Intermediate PJ)

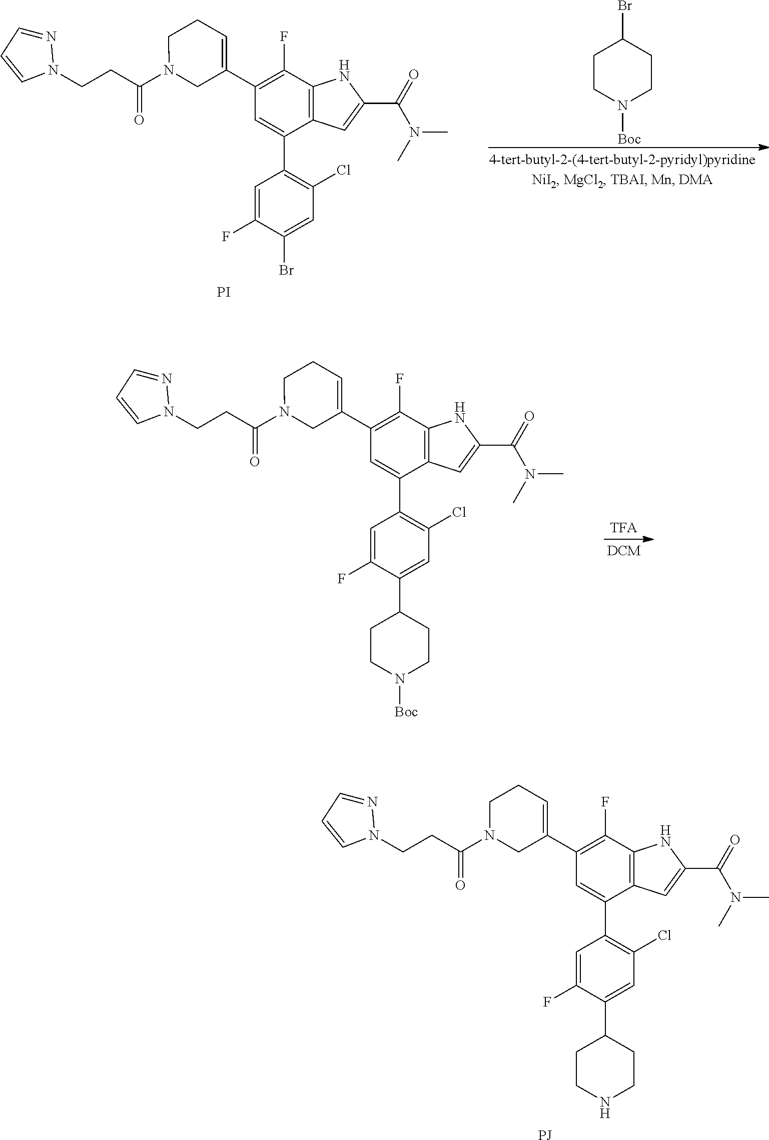 [SMILES: CN(C)C(=O)c1cc2c(-c3cc(F)c(Br)cc3Cl)cc(C3=CCCN(C(=O)CCn4cccn4)C3)c(F)c2[nH]1 ; CC(C)(C)OC(=O)N1CCC(Br)CC1 ; "PI" ; CN(C)C(=O)c1cc2c(-c3cc(F)c(C4CCN(C(=O)OC(C)(C)C)CC4)cc3Cl)cc(C3=CCCN(C(=O)CCn4cccn4)C3)c(F)c2[nH]1 ; CN(C)C(=O)c1cc2c(-c3cc(F)c(C4CCNCC4)cc3Cl)cc(C3=CCCN(C(=O)CCn4cccn4)C3)c(F)c2[nH]1 ; "PJ"]

### Step 1—Tert-butyl 4-(4-(6-(1-(3-(1H-pyrazol-1-yl)propanoyl)-1,2,5,6-tetrahydropyridin-3-yl)-2-(dimethylcarbamoyl)-7-fluoro-1H-indol-4-yl)-5-chloro-2-fluorophenyl)piperidine-1-carboxylate

A mixture of tert-butyl 4-bromopiperidine-1-carboxylate (428 mg, 1.62 mmol, CAS #180695-79-8), pyridine-2,6-dicarboxamidine (162 mg, 810 μmol, HCl), 6-(l-(3-(1H-pyrazol-1-yl)propanoyl)-1,2,5,6-tetrahydropyridin-3-yl)-4-(4-bromo-2-chloro-5-fluorophenyl)-7-fluoro-N,N-dimethyl-1H-indole-2-carboxamide (500 mg, 810 μmol, Intermediate PI), diiodonickel (253 mg, 810 μmol, 43.5 μL), dichloromagnesium (154 mg, 1.62 mmol, 66.5 μL), tetrabutylammonium iodide (299 mg, 810 μmol), and manganese (133 mg, 2.43 mmol, 132 μL) in DMA (8 mL) was degassed and purged with N₂ three times. Then the mixture was stirred at 40° C. for 12 hrs under N₂ atmosphere. On completion, the reaction mixture was quenched with water (30 mL) and extracted with dichloromethane (3×30 mL). The combined organic layers were washed with aqueous NaCl (30 mL) and dried over anhydrous sodium sulfate, filtered and the filtrate was concentrated in vacuo to give the crude residue. The crude residue was purified by reversed-phase HPLC (0.1% FA condition) to give the title compound (110 mg, 19% yield) as a white oil. LC-MS (ESI⁺) m/z 721.4 (M+H)⁺.

### Step 2—6-(1-(3-(1H-pyrazol-1-yl)propanoyl)-1,2,5,6-tetrahydropyridin-3-yl)-4-(2-chloro-5-fluoro-4-(piperidin-4-yl)phenyl)-7-fluoro-N,N-dimethyl-1H-indole-2-carboxamide

To a solution of tert-butyl 4-(4-(6-(1-(3-(1H-pyrazol-1-yl)propanoyl)-1,2,5,6-tetrahydropyridin-3-yl)-2-(dimethylcarbamoyl)-7-fluoro-1H-indol-4-yl)-5-chloro-2-fluorophenyl)piperidine-1-carboxylate (110 mg, 153 μmol) in DCM (3 mL) was added TFA (844 mg, 7.40 mmol, 550 μL). The mixture was then stirred at 25° C. for 1.5 hrs. Upon completion, the reaction mixture was concentrated under reduced pressure to remove solvent to give the title compound (110 mg, TFA) as a white oil. LC-MS (ESI⁺) m/z 621.3 (M+H); ¹H NMR (400 MHz, DMSO-d₆) δ=12.38-11.92 (m, 1H), 8.74-8.55 (m, 1H), 8.43-8.23 (m, 1H), 7.74-7.60 (m, 1H), 7.46-7.29 (m, 3H), 6.99-6.87 (m, 1H), 6.48-6.41 (m, 1H), 6.20-6.19 (m, 1H), 6.20-6.05 (m, 2H), 4.35-4.28 (m, 6H), 4.26-4.22 (m, 2H), 3.62-3.35 (m, 10H), 3.10-2.93 (m, 8H), 2.30-2.20 (m, 2H), 1.98-1.85 (m, 4H).

### 3-(2-Fluoro-4-(piperazin-1-yl)phenyl)piperidine-2,6-dione (Intermediate PK)

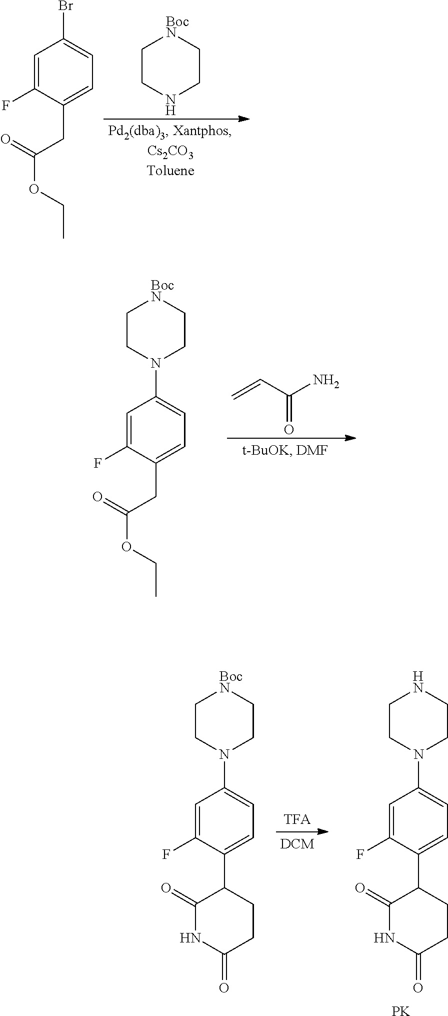

### Step 1—Tert-butyl 4-(4-(2-ethoxy-2-oxoethyl)-3-fluorophenyl)piperazine-1-carboxylate

To a solution of ethyl 2-(4-bromo-2-fluoro-phenyl)acetate (2 g, 8 mmol) in toluene (20 mL) was added tert-butyl piperazine-1-carboxylate (1.43 g. 7.6 mmol), Cs₂CO₃ (7.49 g, 22.9 mmol), Xantphos (886.4 mg, 1.5 mmol) and Pd₂(dba)₃ (701.4 mg, 766 μmol). The mixture was then stirred at 100° C. for 4 hrs. Upon completion, the reaction mixture was concentrated under reduced pressure to remove toluene. The residue was purified by column chromatography (SiO₂, Petroleum ether/Ethyl acetate=20/1 to 10/1) to give the title compound (1.6 g, 3.8 mmol, 51% yield) as a white solid. LC-MS (ESI⁺) m/z 311.0 (M−55)⁺; ¹H NMR (400 MHz, DMSO-d6) δ7.51-7.39 (m, 1H), 7.12 (t, J=8.8 Hz, 1H), 6.72 (d, J=2.4 Hz, 1H), 4.11-4.00 (m, 2H), 3.56 (s, 2H), 3.49-3.40 (m, 4H), 3.16-3.08 (m, 4H), 1.44 (s, 9H), 1.16 (t, J=7.2 Hz, 3H).

### Step 2—Tert-butyl 4-(4-(2,6-dioxopiperidin-3-yl)-3-fluorophenyl)piperazine-1-carboxylate

To a solution of tert-butyl 4-[4-(2-ethoxy-2-oxo-ethyl)-3-fluoro-phenyl]piperazine-1-carboxylate (1.4 g, 3.8 mmol) in DMF (2 mL) was added tBuOK (1 M, 3.8 mL) and prop-2-enamide (271.5 mg, 3.8 mmol, 263.6 μL). The mixture was then stirred at 0° C. for 0.2 hr. Upon completion, the mixture was quenched by addition of HCl (6M) aq (10 mL) at 0° C. The reaction mixture was filtered and concentrated under reduced pressure to give the title compound (800 mg) as a white solid. LC-MS (ESI⁺) m/z 392.2 (M+H)⁺.

### Step 3—3-(2-Fluoro-4-(piperazin-1-yl)phenyl)piperidine-2,6-dione

To a solution of tert-butyl 4-[4-(2,6-dioxo-3-piperidyl)-3-fluoro-phenyl]piperazine-1-carboxylate (800 mg, 2 mmol) in DCM (8 mL) was added TFA (4.61 g, 40.3 mmol, 3 mL). The mixture was then stirred at 25° C. for 1 hr. Upon completion, the reaction mixture was concentrated under reduced pressure to remove DCM. The residue was purified by prep-HPLC (HCl condition) to give the title compound (350 mg, 51% yield, HCl) as a white solid. LC-MS (ESI⁺) m/z 292.1 (M+H)⁺; ¹H NMR (400 MHz, DMSO-d6) δ 10.84 (s, 1H), 7.16 (t, J=8.8 Hz, 1H), 6.93-6.58 (m, 211), 3.99-3.89 (m, 1H), 3.40 (s, 4H), 3.24 (s, 4H), 2.79-2.69 (m, 1H), 2.56-2.52 (m, 2H), 2.23-2.06 (m, 1H), 2.03-1.87 (m, 1H).

### 3-(4-(4-(1-(4-Chloro-2,5-difluorophenyl)piperidin-4-yl)piperazin-1-yl)-2-fluorophenyl)piperidine-2,6-dione (Intermediate PL)

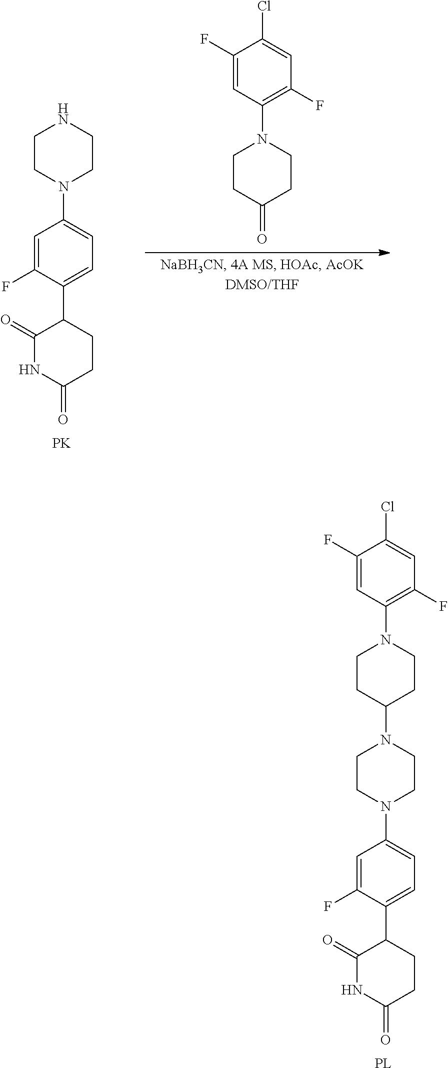

To a solution of 3-(2-fluoro-4-piperazin-1-yl-phenyl)piperidine-2,6-dione (300 mg, 915 μmol, HCl, Intermediate PK) in DMSO (4 mL) THF (1 mL) was added 1-(4-chloro-2,5-difluoro-phenyl)piperidin-4-one (293.6 mg, 1 mmol, FA, synthesized via Steps 1-3 of Intermediate MC), AcOK (269.4 mg. 2.7 mmol), NaBH₃CN (143.7 mg, 2.2 mmol), 4 Å molecular sieves (200 mg, 915 μmol) and AcOH (164.8 mg, 2.7 mmol, 157.1 μL). The mixture was then stirred at 60° C. for 4 hrs. Upon completion, the mixture was quenched with addition water (10 mL) at 25° C. The residue was diluted with water (100 mL) and extracted with EA (50 mL×2). The combined organic layers were washed with brine (100 mL×2). The organic layer was concentrated under reduced pressure to give the title compound (152 mg) as a white solid. LC-MS (ESI⁺) m/z 521.2 (M+H)⁺.

### 3-(2,5-Difluoro-4-(4-oxopiperidin-1-yl)phenyl)piperidine-2,6-dione (Intermediate PM)

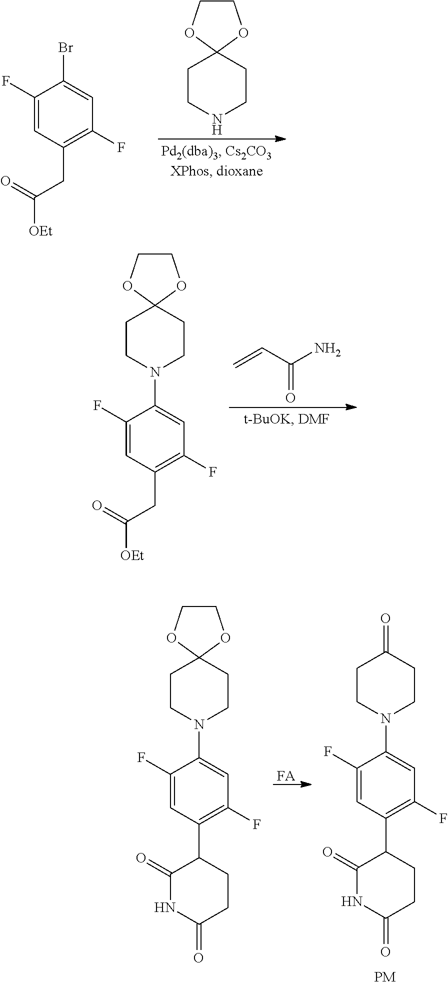

### Step 1—Ethyl 2-(2,5-difluoro-4-(1,4-dioxa-8-azaspiro[4.5]decan-8-yl)phenyl)acetate

A mixture of ethyl 2-(4-bromo-2,5-difluoro-phenyl)acetate (5.00 g, 17.9 mmol, CAS #1807120-48-4), 1,4-dioxa-8-azaspiro[4.5]decane (2.57 g, 17.9 mmol, CAS #177-11-7), XPhos (854 mg, 1.79 mmol,), Pd₂(dba)₃ (820 mg, 896 μmol) and Cs₂CO₃ (17.5 g, 53.8 mmol) in dioxane (50 mL) was degassed and purged with N₂ three times. Then the mixture was stirred at 100° C. for 12 hrs under N₂ atmosphere. On completion, the mixture was filtered and concentrated under reduced pressure to give a residue and purified by column chromatography (SiO₂, Petroleum ether/Ethyl acetate=10/1 to 5/1) to give the title compound (700 mg, 27% yield) as a yellow solid. LC-MS (ESI⁺) m/z 342.2 (M+H)⁺.

### Step 2—3-(2,5-Difluoro-4-(1,4-dioxa-8-azaspiro[4.5]decan-8-yl)phenyl)piperidine-2,6-dione

A mixture of ethyl 2-[4-(1,4-dioxa-8-azaspiro[4.5]decan-8-yl)-2,5-difluoro-phenyl]acetate (3.30 g, 9.67 mmol), prop-2-enamide (824 mg, 11.6 mmol, CAS #9003-05-8), tBuOK (1 M, 11.60 mL) in DMF (30 mL) was degassed and purged with N₂ three times. Then the mixture was stirred at 0° C. for 30 mi under N₂ atmosphere. On completion, the reaction mixture was quenched by addition of 1M HCl (30 mL), filtered to give a filter cake as the title compound (3.4 g) as a yellow solid. LC-MS (ESI⁺) m/z 367.2 (M+H)⁺.

### Step 3—3-(2,5-Difluoro-4-(4-oxopiperidin-1-yl)phenyl)piperidine-2,6-dione

To a solution of 3-[4-(1,4-dioxa-8-azaspiro[4.5]decan-8-yl)-2,5-difluoro-phenyl]piperidine-2,6-dione (3.3 g, 9.0 mmol) was added HCOOH (16 mL). The mixture was then stirred at 70° C. for 12 hrs. On completion, the reaction mixture was concentrated under reduced pressure to remove solvent. The residue was purified by column chromatography (SiO₂, Petroleum ether/Ethyl acetate=1/1 to EA/THF=2/1) to give the title compound (1.2 g, 33% yield) as a yellow solid. LC-MS (ESI⁺) m/z 323.0 (M+H)⁺.

### 3-(4-(4-(4-(4-Bromo-2,3-difluorophenyl)piperazin-1-yl)piperidin-1-yl)-2,5-difluorophenyl)piperidine-2,6-dione (Intermediate PN)

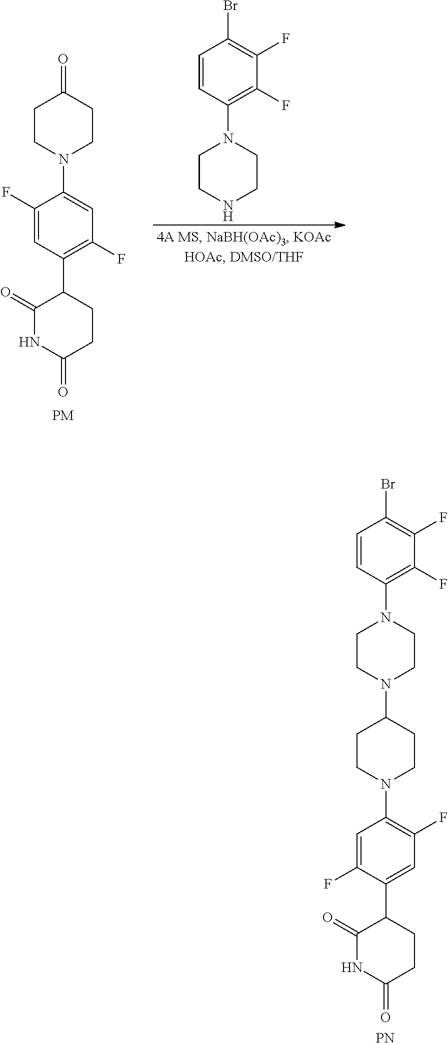

To a solution of 3-[2,5-difluoro-4-(4-oxo-1-piperidyl)phenyl]piperidine-2,6-dione (400 mg, 1.24 mmol, Intermediate PM) and 1-(4-bromo-2,3-difluoro-phenyl)piperazine (584 mg, 1.86 mmol, HCl) in THF (4 mL) and DMSO (4 mL) was added 4 Å molecular sieves (1.24 mmol), HOAc (224 mg, 3.72 mmol) and KOAc (365 mg, 3.72 mmol). The mixture was then stirred at 50° C. for 12 hrs. Then NaBH(OAc)₃ (789 mg, 3.72 mmol) was added, and the mixture was stirred at 25° C. for 2 hrs. On completion, the reaction mixture was filtered to give a residue. To the residue was added (DMF:FA=100:4), then filtered to give the solution. To the solution was added sat. NaHCO₃ to adjust pH to 7-8, then filtered. The filter cake was dried to give the title compound (250 mg, 35% yield) as a yellow solid. LC-MS (ESI⁺) m/z 585.2 (M+H)⁺.

### 7-Bromo-1-(cyclopropylmethyl)-N,N-dimethyl-2-(1,2,5,6-tetrahydropyridin-3-yl)-1H-indole-5-carboxamide (Intermediate PO)

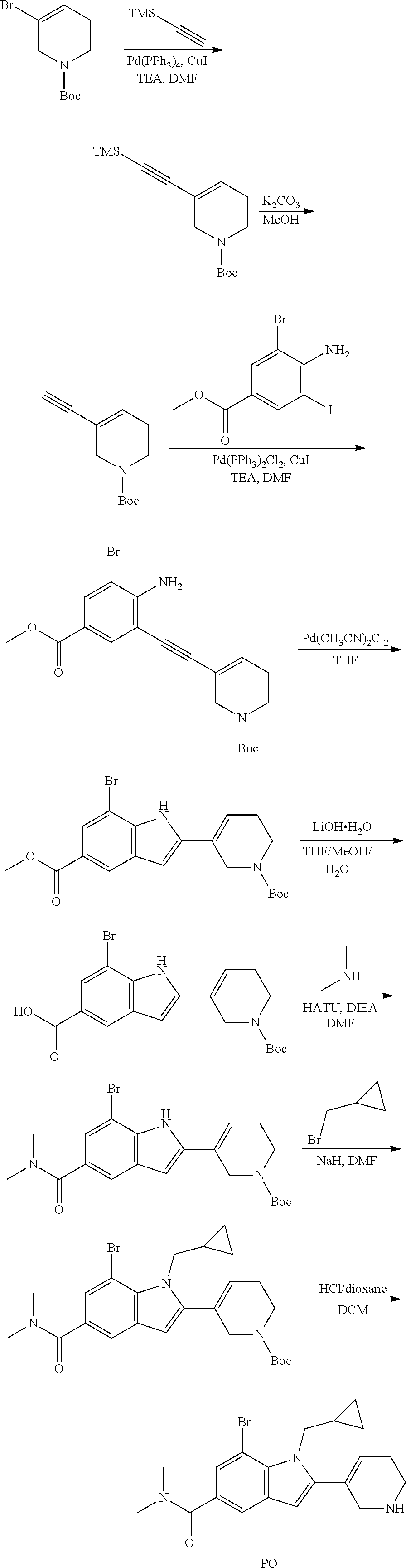

### Step 1—Tert-butyl 3-((trimethylsilyl)ethynyl)-5,6-dihydropyridine-1 (2H)-carboxylate

To a solution of tert-butyl 5-bromo-3,6-dihydro-2H-pyridine-1-carboxylate (13 g, 50 mmol, CAS #1415841-82-5) and ethynyl(trimethyl)silane (48.7 g, 496 mmol, CAS #1066-54-2) in DMF (150 mL) was added Pd(PPh₃)₄ (5.73 g, 4.96 mmol), CuI (944 mg, 4.96 mmol) and TEA (15.1 g, 149 mmol). The mixture was stirred at 50° C. for 12 hrs. On completion, the reaction mixture was diluted with water (100 mL) and extracted with ethyl acetate (3×150 mL). The combined organic layers were washed with brine (200 mL) and dried over anhydrous sodium sulfate, filtered and the filtrate was concentrated in vacuo to give the crude residue. Then the residue was purified by column chromatography (Petroleum ether/Ethyl acetate=1/0) to give the title compound (11 g, 71% yield) as a brown oil. H NMR (400 MHz, DMSO-d₆) δ=6.29-6.16 (m, 1H), 3.81 (br d, J=2.0 Hz, 2H), 3.36 (br t, J=5.2 Hz, 2H), 2.17-2.10 (m, 2H), 1.40 (s, 9H), 0.18-0.12 (m, 9H).

### Step 2—Tert-butyl 3-ethynyl-5,6-dihydropyridine-1 (2H)-carboxylate

To a solution of tert-butyl 5-(2-trimethylsilylethynyl)-3,6-dihydro-2H-pyridine-1-carboxylate (11 g, 39 mmol) in MeOH (120 mL) was added K₂CO₃ (16.3 g, 118 mmol). The mixture was stirred at 25° C. for 2 hrs. Upon completion, the reaction mixture was concentrated under reduced pressure to remove solvent. Then the residue was purified by column chromatography (Petroleum ether/Ethyl acetate=1/0) to give the title compound (6.6 g, 77% yield) as a brown oil. ¹H NMR (400 MHz, DMSO-d₆) δ=6.25 (s, 1H), 3.97-3.87 (m, 1H), 3.82 (d, J=1.9 Hz, 2H), 3.37 (br t, J=5.5 Hz, 2H), 2.17-2.11 (m, 2H), 1.40 (s, 9H).

### Step 3—Tert-butyl 3-((2-amino-3-bromo-5-(methoxycarbonyl)phenyl)ethynyl)-5,6-dihydropyridine-1 (2H)-carboxylate

To a solution of tert-butyl 5-ethynyl-3,6-dihydro-2H-pyridine-1-carboxylate (6.60 g, 31.8 mmol) and methyl 4-amino-3-bromo-5-iodo-benzoate (12.5 g, 35.0 mmol, CAS #1240113-86-3) in DMF (100 mL) was added Pd(PPh₃)₂Cl₂ (2.24 g, 3.18 mmol), CuI (728 mg, 3.82 mmol) and TEA (16.1 g. 159 mmol). The mixture was then stirred at 50° C. for 2 hrs. On completion, the reaction mixture was diluted with water (200 mL) and extracted with ethyl acetate (3×200 mL). The combined organic layers were washed with brine (400 mL) and dried over anhydrous sodium sulfate, filtered and the filtrate was concentrated in vacuo to give the crude residue. Then the residue was purified by column chromatography (Petroleum ether/Ethyl acetate=5/1) to give the title compound (9 g, 62% yield) as a brown oil. LC-MS (ESI⁺) m/z 457.1 (M+Na).

### Step 4—Methyl 7-bromo-2-(1-(tert-butoxycarbonyl)-1,2,5,6-tetrahydropyridin-3-yl)-1H-indole-5-carboxylate

To a solution of tert-butyl 5-[2-(2-amino-3-bromo-5-methoxycarbonyl-phenyl)ethynyl]-3,6-dihydro-2H-pyridine-1-carboxylate (9 g, 21 mmol) in THF (120 mL) was added Pd(CH₃CN)₂Cl₂ (2.68 g, 10.3 nmol). The mixture was stirred at 80° C. for 30 mins. Upon completion, the reaction mixture was concentrated under reduced pressure to remove solvent. Then the residue was purified by column chromatography (Petroleum ether/Ethyl acetate=2/1) to give the title compound (6.4 g, 68% yield) as a white solid. ¹H NMR (400 MHz, DMSO-d₆) δ=11.53-11.47 (m, 1H), 8.27-8.22 (m, 1H), 7.92-7.87 (m, 1H), 6.93-6.86 (m, 1H), 6.80-6.72 (m, 1H), 4.33-4.27 (m, 2H), 3.92-3.88 (m, 3H), 3.55 (t, J=5.6 Hz, 2H), 2.39-2.33 (m, 2H), 1.50 (s, 9H).

### Step 5—7-Bromo-2-(1-(tert-butoxycarbonyl)-1,2,5,6-tetrahydropyridin-3-yl)-1H-indole-5-carboxylic acid

To a solution of methyl 7-bromo-2-(1-tert-butoxycarbonyl-3,6-dihydro-2H-pyridin-5-yl)-1H-indole-5-carboxylate (6.4 g, 15 mmol) in THF (80 mL) MeOH (20 mL) and H₂O (20 mL) was added LiOH·H2O (1.54 g, 36.8 mmol). The mixture was then stirred at 40° C. for 2 hrs. On completion, the reaction mixture was quenched with HCl (10 mL) and extracted with ethyl acetate (3×50 mL). The combined organic layers were washed with brine (100 mL) and dried over anhydrous sodium sulfate, filtered and the filtrate was concentrated in vacuo to give the title compound (6.9 g) as a yellow solid. LC-MS (ESI⁺) m/z 421.1 (M+H)⁺.

### Step 6—Tert-butyl 3-(7-bromo-5-(dimethylcarbamoyl)-1H-indol-2-yl)-5,6-dihydropyridine-1 (2H)-carboxylate

To a solution of 7-bromo-2-(1-tert-butoxycarbonyl-3,6-dihydro-2H-pyridin-5-yl)-1H-indole-5-carboxylic acid (1.8 g, 4.3 mmol) in DMF (15 mL) was added HATU (1.79 g, 4.70 mmol), DIEA (2.76 g, 21.4 mmol, 3.72 mL) and N-methylmethanamine (418.09 mg, 5.13 mmol, HCl, CAS #124-40-3). Upon completion, the mixture was triturated with water (100 mL) and filtered to give title compound (1.7 g, 87% yield) as a white solid. LC-MS (ESI+) m/z 448.1 (M+H)+.

### Step 7— Tert-butyl 3-(7-bromo-1-(cyclopropylmethyl)-5-(dimethylcarbamoyl)-1H-indol-2-yl)-5,6-dihydropyridine-1 (2H)-carboxylate

To a solution of tert-butyl 5-[7-bromo-5-(dimethylcarbamoyl)-1H-indol-2-yl]-3,6-dihydro-2H-pyridine-1-carboxylate (1.7 g, 3.8 mmol) in DMF (15 mL) was added NaH (227 mg, 5.69 mmol, 60% dispersion in mineral oil) under N₂ atmosphere at 0° C., then KI (63.0 mg, 379 μmol) and bromomethylcyclopropane (768 mg, 5.69 mmol. CAS #7051-34-5) was added. The mixture was then stirred at 0-25° C. for 2 hrs. On completion, the reaction mixture was quenched with NH4Cl (20 mL) and extracted with ethyl acetate/dichloromethane (3×50 mL). The combined organic layers were washed with brine (100 mL) and dried over anhydrous sodium sulfate, filtered and the filtrate was concentrated in vacuo to give the crude residue. Then the residue was purified by column chromatography (DCM/THF=2/1) to give the title compound (1.2 g, 61% yield) as a yellow oil. ¹H NMR (400 MHz, DMSO-d₆) δ=7.44 (d, J=1.6 Hz, 1H), 7.22-7.19 (m, 1H), 6.41-6.37 (m, 1H), 5.88 (s, 1H), 4.30-4.23 (m, 2H), 3.90 (s, 2H), 3.36-3.28 (m, 2H), 2.81-2.78 (m, 6H), 2.11 (d, J=2.4 Hz, 2H), 1.24 (s, 9H), 0.90-0.83 (m, 1H), 0.17 (d, J=7.2 Hz, 2H), −0.01 (q, J=4.8 Hz, 2H).

### Step 8—7-Bromo-1-(cyclopropylmethyl)-N,N-dimethyl-2-(1,2,5,6-tetrahydropyridin-3-yl)-1H-indole-5-carboxamide

To a solution of tert-butyl 5-[7-bromo-1-(cyclopropylmethyl)-5-(dimethylcarbamoyl) indol-2-yl]-3,6-dihydro-2H-pyridine-1-carboxylate (1.2 g, 2.4 mmol in DCM (12 mL) was added HCl/dioxane (2 M, 15 mL). The mixture was then stirred at 25° C. for 30 mins. Upon completion, the reaction mixture was concentrated under reduced pressure to remove solvent to give the title compound (1.3 g, HCl) as a yellow oil. LC-MS (ESI⁺) m/z 402.1 (M+H)⁺.

### 2-(1-(3-(1H-1,2,3-triazol-1-yl)propanoyl)-1,2,5,6-tetrahydropyridin-3-yl)-7-bromo-1-(cyclopropylmethyl)-N,N-dimethyl-1H-indole-5-carboxamide (Intermediate PP)

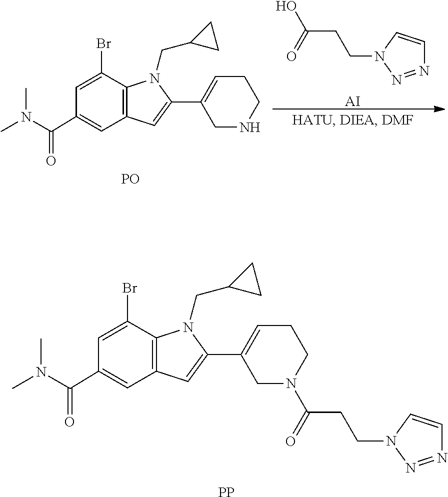 [SMILES: CN(C)C(=O)c1cc(Br)c2c(c1)cc(C1=CCCNC1)n2CC1CC1 ; O=C(O)CCn1ccnn1 ; "AI" ; "PO" ; CN(C)C(=O)c1cc(Br)c2c(c1)cc(C1=CCCN(C(=O)CCn3ccnn3)C1)n2CC1CC1 ; "PP"]

To a solution of 3-(triazol-1-yl)propanoic acid (502 mg, 3.56 mmol, Intermediate AL) in DMF (13 mL) was added HATU (1.24 g, 3.26 mmol), DIEA (1.91 g, 14.8 mmol) and 7-bromo-1-(cyclopropylmethyl)-N,N-dimethyl-2-(1,2,3,6-tetrahydropyridin-5-yl) indole-5-carboxamide (1.3 g, 3.0 mmol. Intermediate PO). The mixture was then stirred at 25° C. for 10 mins. On completion, the reaction mixture was diluted with water (10 mL) and extracted with ethyl acetate (3×20 mL). The combined organic layers were washed with brine (40 mL) and dried over anhydrous sodium sulfate, filtered and the filtrate was concentrated in vacuo to give the crude residue. Then the residue was purified by column chromatography (DCM/THF=2/1) to give the title compound (1.34 g, 65% yield) as a yellow oil. LC-MS (ESI+) m/z 525.2 (M+H)+.

### 6-(1-(3-(1H-1,2,3-triazol-1-yl)propanoyl)-1,2,5,6-tetrahydropyridin-3-yl)-N-ethyl-7-fluoro-N-methyl-4-(4,4,5,5-tetramethyl-1,3,2-dioxaborolan-2-yl)-1H-indole-2-carboxamide (Intermediate PQ)

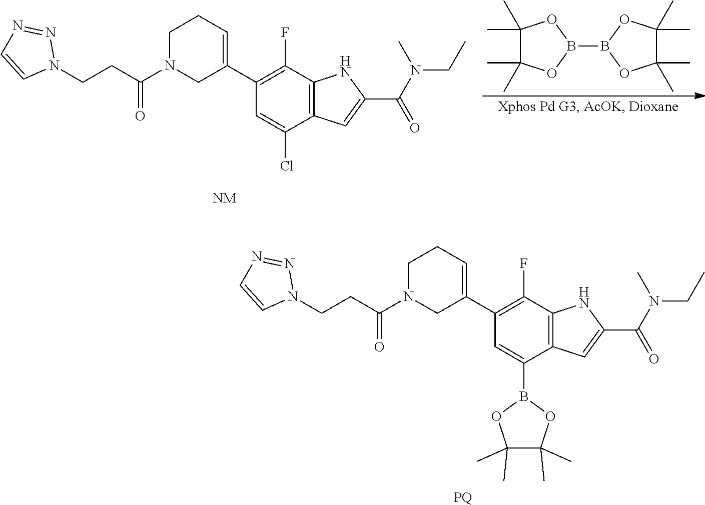

A mixture of 4-chloro-N-ethyl-7-fluoro-N-methyl-6-[1-[3-(triazol-1-yl)propanoyl]-3,6-dihydro-2H-pyridin-5-yl]-1H-indole-2-carboxamide (2 g, 3 mmol, Intermediate NM), 4,4,5,5-tetramethyl-2-(4,4,5,5-tetramethyl-1,3,2-dioxaborolan-2-yl)-1,3,2-dioxaborolane (1.44 g, 5.67 mmol, CAS #73183-34-3). XPhos Pd G₃ (239.78 mg, 283.28 μmol), and AcOK (834.04 mg, 8.50 mmol) in dioxane (20 mL) was degassed and purged with N₂ three times at 20° C. Then the mixture was stirred at 80° C. for 1 hr under N₂ atmosphere. Upon completion, the reaction mixture was concentrated under reduced pressure to remove the solvent. The residue was purified by column chromatography (SiO₂, EA:THF=40/1 to 5/1) to give the title compound (1.2 g, 69% yield) as a brown solid. LC-MS (ESI⁺) m/z 551.3 (M+H)⁺; ¹H NMR (400 MHz, DMSO-d₆) δ=12.14-12.03 (m, 1H), 8.15-8.08 (m, 1H), 7.73-7.65 (m, 1H), 7.37-7.30 (m, 1H), 7.06-7.00 (m, 1H), 6.04 (s, 1H), 4.66-4.60 (m, 2H), 4.33-4.26 (m, 2H), 3.68-3.62 (m, 1H), 3.54 (s, 4H), 3.13-3.02 (m, 4H), 2.26 (s, 2H), 1.35-1.29 (m, 12H), 1.22-1.16 (m, 3H).

The following four compounds were synthesized as described in patent application number PCT/US2023/077741.

### Methyl 5-(4-bromobenzoyl)-4-(3,6-difluoro-2-methylphenyl)-1-methyl-1H-pyrrole-3-carboxylate (Intermediate PR)

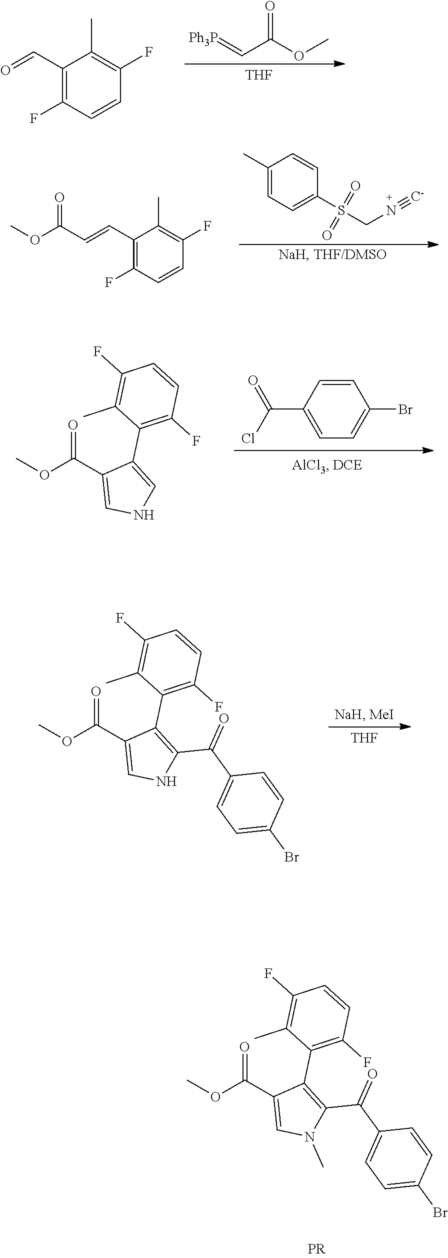

### Step 1—Methyl (E)-3-(3,6-difluoro-2-methylphenyl)acrylate

A solution of 3,6-difluoro-2-methylbenzaldehyde (5.00 g. 32.0 mmol. CAS #1378525-21-3) and methyl 2-(triphenyl-lambda5-phosphanylidene)acetate (12.85 g, 38.43 mmol) in THF (60 mL) was stirred for 2 h at rt. On completion, the resulting mixture was diluted with H₂O (100 mL) and extracted with EtOAc (3×200 mL). The combined organic layers were washed with brine (2×100 mL), and dried over anhydrous Na₂SO₄. After filtration, the filtrate was concentrated under reduced pressure. The residue was purified by silica gel column chromatography, eluted with PE/EA (8:1) to afford the title compound (6.30 g, 93% yield) as a white solid. ¹H NMR (400 MHz, DMSO-d₆) δ 7.63 (d, J=16.3 Hz, 1H), 7.35-7.27 (m, 1H), 7.27-7.11 (m, 1H), 6.49 (dd, J=16.4, 1.2 Hz, 1H), 3.76 (s, 3H), 2.29 (d, J=2.3 Hz, 3H).

### Step 2—Methyl 4-(3,6-difluoro-2-methylphenyl)-1H-pyrrole-3-carboxylate

To a stirred solution of methyl (E)-3-(3,6-difluoro-2-methylphenyl)acrylate (13.00 g, 61.26 mmol) and TosMIC (15.64 g, 80.12 mmol) in THF (70 mL) and DMSO (70 mL) were added NaH (2.31 g, 96.1 mmol) in portions at 0° C. The resulting mixture was stirred overnight at rt. On completion, the reaction was quenched with water/ice at 0° C. The resulting mixture was extracted with EtOAc (3×300 mL). The combined organic layers were washed with brine (2×100 mL), and dried over anhydrous Na₂SO₄. After filtration, the filtrate was concentrated under reduced pressure. The residue was purified by silica gel column chromatography, eluted with PE/EA (3:1), to afford the title compound (16.00 g, 80% yield) as a white solid. ¹H NMR (400 MHz, DMSO-d₆) δ 11.68 (s, 1H), 7.58-7.52 (m, 1H), 7.18-7.08 (m, 1H), 7.07-6.97 (m, 1H), 6.86 (t, J=2.3 Hz, 1H), 3.56 (s, 3H), 2.00 (d, J=2.6 Hz, 3H). LC/MS (ESI, m/z): [(M+1)]=252.1.

### Step 3—Methyl 5-(4-bromobenzoyl)-4-(3,6-difluoro-2-methylphenyl)-1H-pyrrole-3-carboxylate

To a stirred mixture of methyl 4-(3,6-difluoro-2-methylphenyl)-1H-pyrrole-3-carboxylate (2.5 g, 9.9 mmol) and AlCl₃ (3.98 g, 29.9 mmol) in DCE (100 mL) were added 4-bromo-benzoyl chloride (2.62 g, 11.9 mmol) in portions at 0° C. under air atmosphere. The resulting mixture was stirred for 5 h at 50° C. under air atmosphere. On completion, the mixture was purified by silica gel column chromatography, eluted with PE/EA (15:1), to afford the title compound (3.5 g, 81% yield) as a yellow oil. ¹H NMR (400 MHz, DMSO-d₆) δ 12.86 (s, 1H), 7.90-7.85 (m, 1H), 7.45-7.37 (m, 211), 7.29 (m, 2H), 7.03-6.93 (m, 1H), 6.84-6.73 (m, 1H), 3.60 (s, 3H), 1.85 (d, J=2.5 Hz, 3H). LC/MS (ESI, m/z): [(M+1)]⁺=434.9.

### Step 4—Methyl 5-(4-bromobenzoyl)-4-(3,6-difluoro-2-methylphenyl)-1-methyl-1H-pyrrole-3-carboxylate

To a stirred mixture of methyl 5-(4-bromobenzoyl)-4-(3,6-difluoro-2-methylphenyl)-1H-pyrrole-3-carboxylate (3.4 g, 7.8 mmol) in THF (50 mL) was added NaH (0.63 g, 16 mmol, 60% dispersion in mineral oil) in portions at 0° C. under N₂ atmosphere. The resulting mixture was stirred for 30 min at rt under N₂ atmosphere. To the above mixture was added CH₃I (1.67 g, 11.7 mmol) dropwise over 2 min at rt. The resulting mixture was stirred for additional 2 h at rt. On completion, the mixture was diluted with water (50 mL) and concentrated under reduced pressure. The residue was dissolved in DMF (5 mL). The mixture was purified by reverse phase Flash chromatography (Column: WelFlash™ C18-I, 20-40 um, 330 g; Eluent A: Water (plus 10 mmol/L, NH₄ICO₃); Eluent B: ACN; Gradient: 25%-55% B in 25 min: Flow rate: 60 mL/min; Detector: 220/254 nm; desired fractions were collected at 50% B) and concentrated under reduced pressure to afford the title compound (800 mg, 23% yield) as a brown oil. ¹H NMR (400 MHz, DMSO-d₆) δ 8.05 (s, 1H), 7.42-7.35 (m, 2H), 7.35-7.27 (m, 2H), 6.96-6.86 (m, J=9.1, 4.5 Hz, 1H), 6.78-6.61 (m, 1H), 3.94 (s, 3H), 3.59 (s, 3H), 1.86 (d, J=2.5 Hz, 3H). LC/MS (ESI, m/z): [(M+1)]*=447.1.

### (R)-1-(3-chlorophenyl)-2-(methylsulfonyl)ethan-1-amine (Intermediate PS)

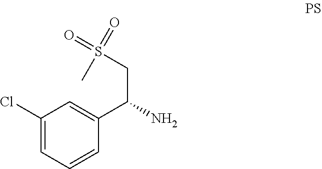

### (3aR,7aR)-4-(3-fluorophenyl)-octahydropyrrolo[3,2-b]pyridine hydrochloride (Intermediate PT)

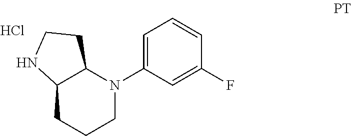 [SMILES: Cl ; Fc1cccc(N2CCC[C@H]3NCC[C@H]32)c1]

### 5-[4-(8-Bromooct-1-yn-1-yl)benzoyl]-N-[(1R)-1-(3-chlorophenyl)-2-methanesulfonylethyl]-4-(3,6-difluoro-2-methylphenyl)-1-methylpyrrole-3-carboxamide (Intermediate PU)

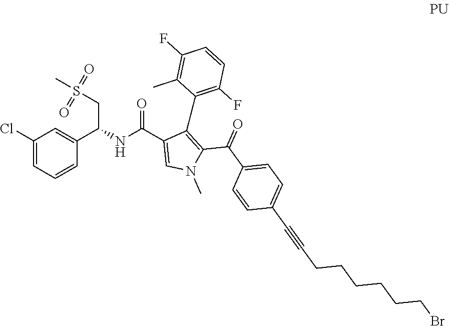 [SMILES: Cc1c(F)ccc(F)c1-c1c(C(=O)N[C@@H](CS(C)(=O)=O)c2cccc(Cl)c2)cn(C)c1C(=O)c1ccc(C#CCCCCCCBr)cc1]

### 7-Fluoro-4-[2-methoxy-4-(piperazin-1-yl)phenyl]-N,N-dimethyl-6-{1-[3-(1,2,3-triazol-1-yl)propanoyl]-5,6-dihydro-2H-pyridin-3-yl}-1H-indole-2-carboxamide (Intermediate PV)

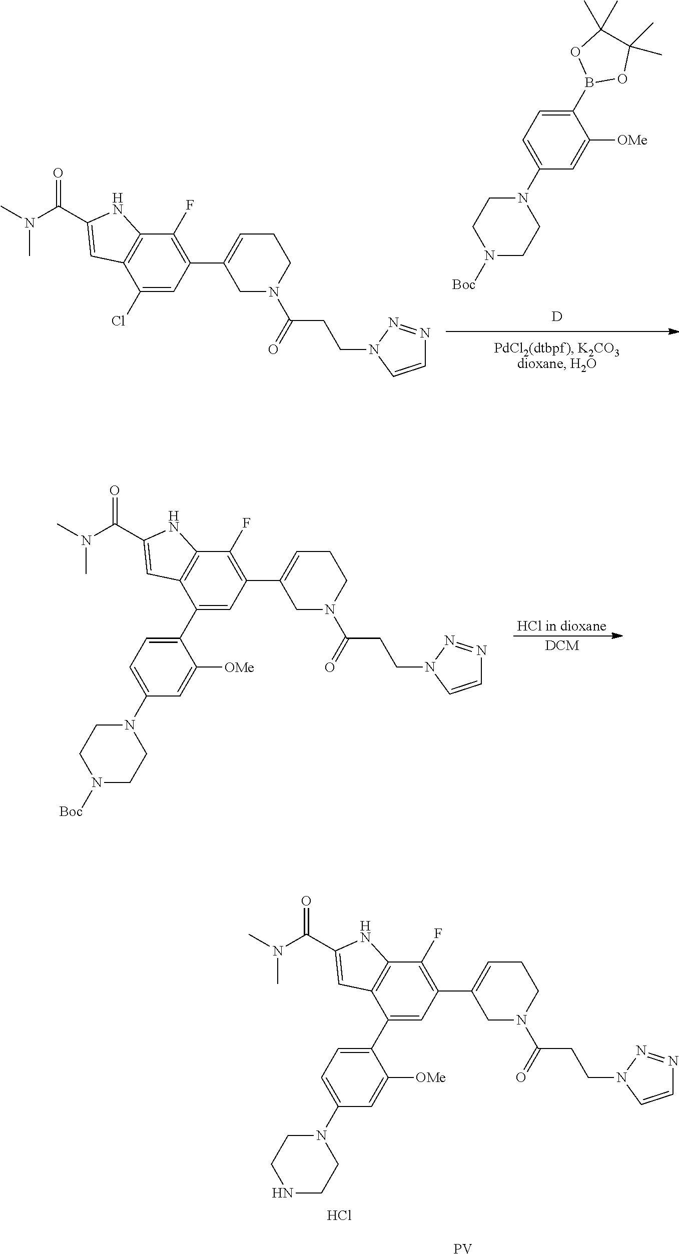

### Step 1—Tert-butyl 4-{4-[2-(dimethylcarbamoyl)-7-fluoro-6-{1-[3-(1,2,3-triazol-1-yl)propanoyl]-5,6-dihydro-2H-pyridin-3-yl}-1H-indol-4-yl]-3-methoxyphenyl}piperazine-1-carboxylate

To a stirred solution of 4-chloro-7-fluoro-N,N-dimethyl-6-{1-[3-(1,2,3-triazol-1-yl)propanoyl]-5,6-dihydro-2H-pyridin-3-yl}-1H-indole-2-carboxamide (700 mg, 1.57 mmol, synthesized via Step 1 of Intermediate AK) and tert-butyl 4-[3-methoxy-4-(4,4,5,5-tetramethyl-1,3,2-dioxaborolan-2-yl)phenyl]piperazine-1-carboxylate (789.85 mg, 1.888 mmol, Intermediate D) in 1,4-dioxane (10 mL)/H₂O (2 mL) were added K₂CO₃ (434.90 mg, 3.146 mmol) and Pd(DtBPF)Cl₂ (102.55 mg, 0.157 mmol) at rt under air atmosphere. The resulting mixture was stirred for 2 h at 80° C. under nitrogen atmosphere. On completion, the mixture was cooled to rt and concentrated under reduced pressure. The residue was purified by reverse-phase flash chromatography (column, C18 silica gel; mobile phase, MeCN in Water (10 mmol/L NH₄HCO₃), 52% to 82% gradient in 25 min; detector, UV 254 nm: desired fractions were collected at 60% B and concentrated under reduced pressure) to afford the title compound (580 mg, 53% yield) as a yellow oil. ¹H NMR (400 MHz, Chloroform-d) δ 9.49-9.40 (m, 1H), 7.78 (d, J=4.6 Hz, 1H), 7.68 (d, J=2.9 Hz, 1H), 7.29 (s, 1H), 7.01-6.92 (m, 1H), 6.71-6.66 (m, 1H), 6.64-6.61 (m, 1H), 6.21-6.10 (m, 1H), 4.86-4.77 (m, 2H), 4.54-4.47 (m, 1H), 4.30 (d, J=2.6 Hz, 1H), 3.86-3.75 (m, 4H), 3.66 (s, 1H), 3.66-3.55 (m, 4H), 3.32-3.22 (m, 6H), 3.17 (s, 1H), 3.12-3.02 (m, 2H), 2.41-2.33 (m, 2H), 2.03 (s, 2H), 1.68-1.57 (m, 2H), 1.52 (s, 9H); LC/MS (ESI, m/z): [(M+H)]⁺=701.5.

### Step 2—7-Fluoro-4-[2-methoxy-4-(piperazin-1-yl)phenyl]-N,N-dimethyl-6-{1-[3-(1,2,3-triazol-1-yl)propanoyl]-5,6-dihydro-2H-pyridin-3-yl}-1H-indole-2-carboxamide hydrochloride

To a stirred solution of tert-butyl 4-{4-[2-(dimethylcarbamoyl)-7-fluoro-6-{1-[3-(1,2,3-triazol-1-yl)propanoyl]-5,6-dihydro-2H-pyridin-3-yl}-1H-indol-4-yl]-3-methoxyphenyl}piperazine-1-carboxylate (580 mg, 0.828 mmol) in DCM (6 mL) was added 4 M HCl (gas) in 1,4-dioxane (6 mL, 24 mmol) at rt under air atmosphere. The resulting mixture was stirred for 2 h at rt under air atmosphere. On completion, the mixture was concentrated under reduced pressure. The residue was purified by trituration with Et₂O (15 mL) and dried under reduced pressure to afford the title compound (600 mg) as an orange solid. LC/MS (ESI, m/z): [(M+H)]⁺=601.6.

### 3-(1-{12-[(3aR,7aR)-4-(3-fluorophenyl)-hexahydro-2H-pyrrolo[3,2-b]pyridin-1-yl]pyridin-4-yl}piperidin-4-yl)propanal (Intermediate PW)

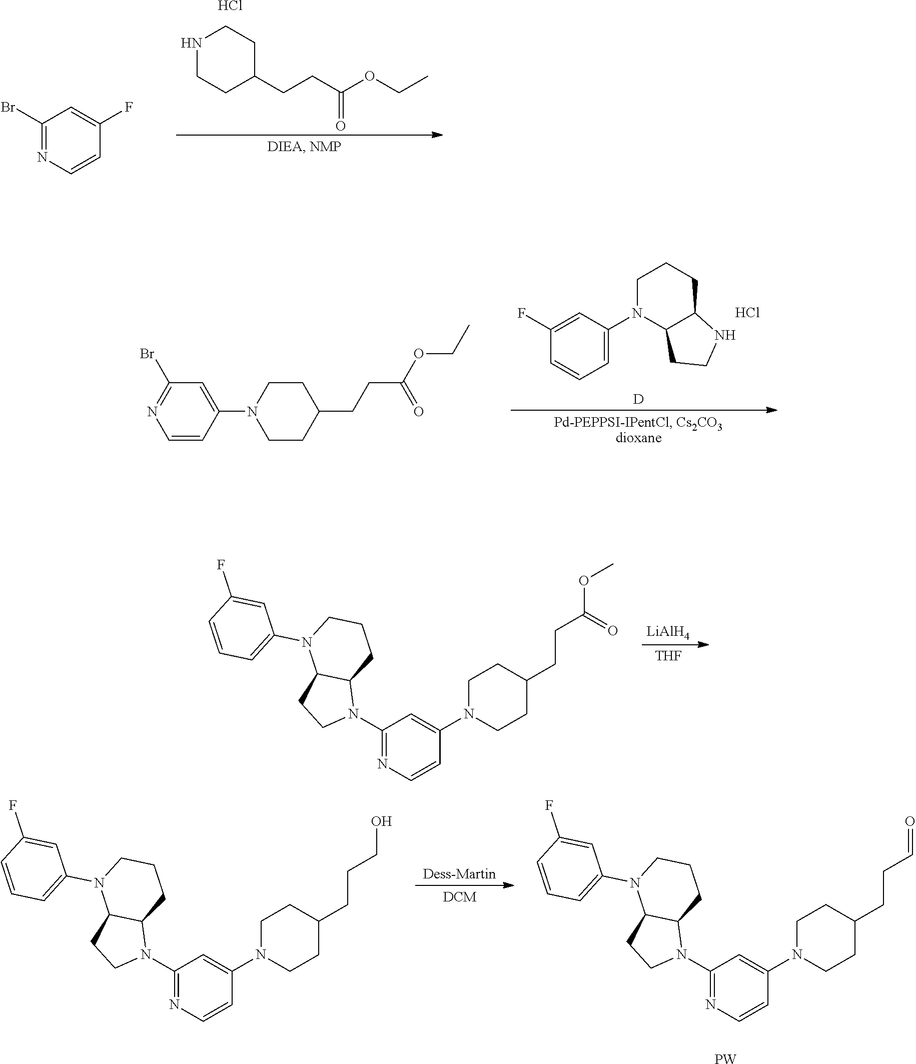

### Step 1—Ethyl 3-[I-(2-bromopyridin-4-yl)piperidin-4-yl]propanoate

To a stirred solution of ethyl 3-(piperidin-4-yl)propanoate hydrochloride (2 g, 9 mmol, CAS #473987-06-3) and DIEA (3.50 g, 27.1 mmol) in NMP (20 mL) was added 2-bromo-4-fluoropyridine (1.59 g, 9.02 mmol, CAS #357927-50-5) at rt under air atmosphere. The resulting mixture was stirred for 20 min at 130° C. under air atmosphere. On completion, H₂O (40 mL) was added and the aqueous layer was extracted with EtOAc (3×20 mL). The resulting mixture was concentrated under reduced pressure. The residue was purified by reverse-phase flash chromatography (column, C18 silica gel; mobile phase, MeCN in Water (10 mmol/L NH4HCO3), 36% to 66% gradient in 30 min; detector, UV 254 nm; desired fractions were collected at 65% B) and concentrated under reduced pressure to afford the title compound (700 mg, 23% yield) as a yellow oil. LC/MS (ESI, m/z): [(M+H)]⁺=341.1.

### Step 2—Ethyl 3-(1-{2-[(3aR,7aR)-4-(3-fluorophenyl)-hexahydro-2H-pyrrolo[3,2-b]pyridin-1-yl]pyridin-4-yl}piperidin-4-yl)propanoate

To a stirred solution of (3aR,7aR)-4-(3-fluorophenyl)-octahydropyrrolo[3,2-b]pyridine hydrochloride (526.67 mg, 2.051 mmol, Intermediate PT) and ethyl 3-[1-(2-bromopyridin-4-yl)piperidin-4-yl]propanoate (700 mg, 2.05 mmol) in 1,4-dioxane (7 mL) were added Cs₂CO₃ (1.34 g. 4.10 mmol) and Pd-PEPPSI-IPentCl 2-methylpyridine o-picoline (172.55 mg, 0.205 mmol) at rt under air atmosphere. The resulting mixture was stirred for 16 h at 100° C. under nitrogen atmosphere. On completion, the mixture was cooled to rt and concentrated under reduced pressure. The residue was purified by reverse-phase flash chromatography (column, C18 silica gel; mobile phase, MeCN in Water (0.1% TFA), 35% to 65% gradient in 30 min; detector, UV 254 nm; desired fractions were collected at 51% B) and concentrated under reduced pressure to afford the title compound (850 mg. 86% yield) as a brown yellow solid. LC/MS (ESI, m/z): [(M+H)]⁺=481.4.

### Step 3—3-(1-{2-[(3aR,7aR)-4-(3-fluorophenyl)-hexahydro-2H-pyrrolo[3,2-b]pyridin-1-yl]pyridin-4-yl}piperidin-4-yl)propan-1-ol

To a stirred solution of ethyl 3-(1-{2-[(3aR,7aR)-4-(3-fluorophenyl)-hexahydro-2H-pyrrolo[3,2-b]pyridin-1-yl]pyridin-4-yl}piperidin-4-yl)propanoate (850 mg, 1.77 mmol) in THF (0.5 mL) was added LiAlH₄ (1.77 mL, 3.54 mmol) at 0° C. under nitrogen atmosphere. The resulting mixture was stirred for 1 h at rt under nitrogen atmosphere. On completion, the reaction was quenched with sat. NH4Cl (aq.) at 0° C. The resulting mixture was extracted with EtOAc (3×20 mL). The combined organic layers were washed with brine (20 mL), and dried over anhydrous Na₂SO₄. After filtration, the filtrate was concentrated under reduced pressure to afford the title compound (700 mg, 90% yield) as a yellow solid. LC/MS (ESI, m/z): [(M+H)]⁺=439.4.

### Step 4—3-(1-{2-[(3aR,7aR)-4-(3-fluorophenyl)-hexahydro-2H-pyrrolo[3,2-b]pyridin-1-yl]pyridin-4-yl}piperidin-4-yl)propanal

To a stirred solution of 3-(1-{2-[(3aR,7aR)-4-(3-fluorophenyl)-hexahydro-2H-pyrrolo[3,2-b]pyridin-1-yl]pyridin-4-yl}piperidin-4-yl)propan-1-ol (500 mg, 1.14 mmol) in DCM (10 mL) was added 1,1-bis(acetyloxy)-3-oxo-3H-llambda5,2-benziodaoxol-1-yl acetate (725.29 mg, 1.710 mmol) at rt under air atmosphere. The resulting mixture was stirred for 4 h at rt under air atmosphere. On completion, the mixture was concentrated under reduced pressure. The residue was purified by reverse-phase flash chromatography (column, C18 silica gel; mobile phase, MeCN in Water (0.1% CH₃COOH), 40% to 70% gradient in 30 min; detector, UV 254 nm; desired fractions were collected at 56% B) and concentrated under reduced pressure. The crude product (400 mg) was then purified by Prep-TLC (CH₂Cl2/MeOH 5:1) to afford the title compound (95 mg, 19% yield) as a yellow solid. LC/MS (ESI, m/z): [(M+H)]⁺=437.2.

### 6-[1-(2-Bromopyridin-4-yl)piperidin-4-yl]hexan-1-ol (Intermediate PX)

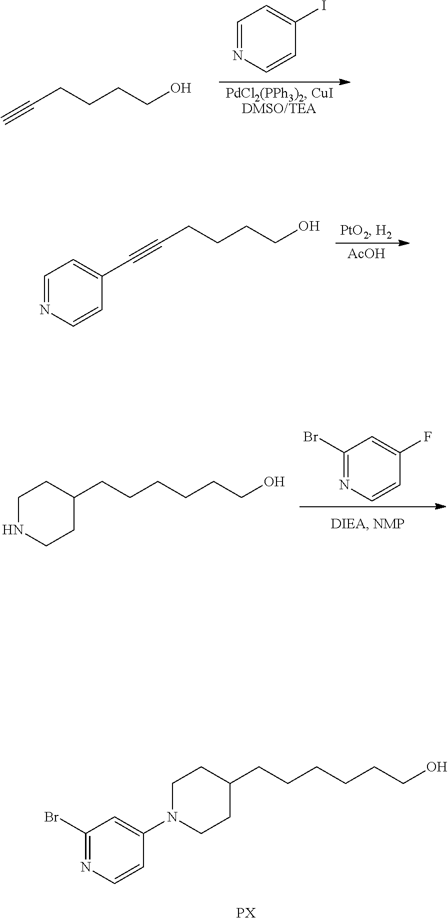

### Step 1—6-(Pyridin-4-yl)hex-5-yn-1-ol

To a stirred mixture of hex-5-yn-1-ol (5 g, 50 mmol) and PPTS (2.56 g, 10.2 mmol) in DCM (100 mL) was added dihydropyran (17.14 g, 203.8 mmol) at rt under nitrogen atmosphere. The resulting mixture was stirred for 1 h at 80° C. under nitrogen atmosphere. On completion, the mixture was extracted with EtOAc (3×600 mL). The combined organic layers were washed with water (2×1 L), and dried over anhydrous Na₂SO₄. After filtration, the filtrate was concentrated under reduced pressure. The residue was dissolved in DMSO (20 mL). The mixture was purified by reverse phase flash chromatography (Column: WelFlash™ C18-I, 20-40 urn, 330 g; Eluent A: Water (plus 10 mmol/L NH4HCO₃); Eluent B: ACN; Gradient: 5%-50% B in 40 min; Flow rate: 80 mL/min; Detector: 220/254 nm; desired fractions were collected at 25% B and concentrated under reduced pressure) to afford the title compound (5 g, 56% yield) as a black oil. 1H NMR (300 MHz, Chloroform-d) δ 8.74 (s, 2H), 7.31 (d, J=12.0 Hz, 2H), 3.73 (h, J=3.4 Hz, 2H), 2.53-2.43 (m, 1H), 1.92 (s, 1H), 1.82-1.69 (m, 4H). LC/MS (ESI, m/z): [(M+1)]=176.2.

### Step 2—6-(Piperidin-4-yl)hexan-1-ol

To a stirred solution of 6-(pyridin-4-yl)hex-5-yn-1-ol (4 g, 20 mmol) in AcOH (100 mL) were added PtO₂ (0.52 g, 2.28 mmol) in portions at rt under nitrogen atmosphere. The resulting mixture was stirred for 24 h at rt under hydrogen atmosphere. On completion, the mixture was filtered, the filter cake was washed with MeOH (5×30 mL), and the filtrate was concentrated under reduced pressure. The mixture was basified to pH=10 with saturated Na₂CO₃ (aq.). The resulting mixture was extracted with EtOAc (6×300 mL). The combined organic layers were washed with brine (3×1000 mL), dried over anhydrous Na₂SO₄, filtered, and the filtrate was concentrated under reduced pressure to give the title compound (3.1 g, 73% yield) as a light yellow oil. ¹H NMR (300 MHz, Chloroform-d) δ 3.59 (t, J=6.6 Hz, 2H), 3.00 (s, 4H), 2.09-1.84 (m, 1H), 1.40-0.98 (m, 15H). LC/MS (ESI, m/z): [(M+1)]⁺=186.2.

### Step 3—6-[1-(2-Bromopyridin-4-yl)piperidin-4-yl]hexan-1-ol

To a stirred solution of 6-(piperidin-4-yl)hexan-1-ol (3 g, 20 mmol) and DIEA (6.28 g, 48.6 mmol) in NMP (20 mL) was added 2-bromo-4-fluoropyridine (3.42 g. 19.4 mmol) dropwise at rt under nitrogen atmosphere. The resulting mixture was stirred for 1 h at 130° C. under nitrogen atmosphere. On completion, the mixture was purified by reverse phase flash chromatography (Column: WelFlash™ C18-I, 20-40 um, 330 g; Eluent A: Water (plus 10 mmol/L NH₄HCO₃); Eluent B: ACN; Gradient: 35%-75% B in 40 min; Flow rate: 80 mL/min; Detector: 220/254 nm; desired fractions were collected at 55% B and concentrated under reduced pressure) to afford the title compound (3.1 g, 56% yield) as a yellow oil. ¹H NMR (300 MHz, Chloroform-d) δ 7.93 (d, J=6.1 Hz, 1H), 6.79 (d, J=2.4 Hz, 1H), 6.64-6.46 (m, 1H), 3.86-3.82 (m, 1H), 3.80-3.76 (m, 1H), 3.64 (t, J=6.6 Hz, 2H), 2.90 (d, J=2.8 Hz, 1H), 2.85 (d, J=2.7 Hz, 1H), 2.81 (d, J=2.8 Hz, 1H), 1.89-1.78 (m, 3H), 1.75 (d, J=3.5 Hz, 2H), 1.64-1.43 (m, 2H), 1.41-1.30 (m, 5H), 1.30-1.11 (m, 3H); LC/MS (ESI, m/z): [(M+1)]⁺=342.0.

### 6-(1-{12-[(3aR,7aR)-4-(3-fluorophenyl)-hexahydro-2H-pyrrolo[3,2-b]pyridin-1-yl]pyridin-4-yl}piperidin-4-yl)hexanal (Intermediate PY)

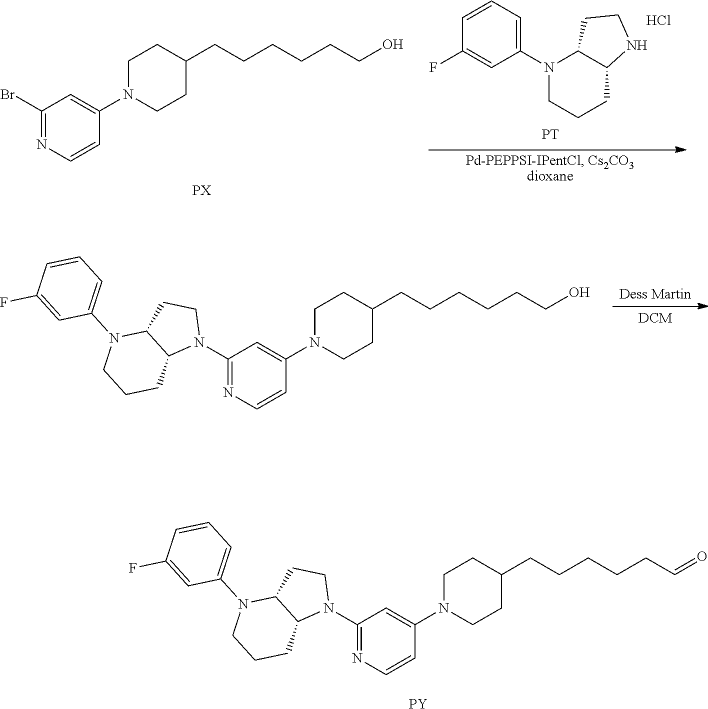

### Step 1—6-(1-{12-[(3aR,7aR)-4-(3-fluorophenyl)-hexahydro-2H-pyrrolo[3,2-b]pyridin-1-yl]pyridin-4-yl}piperidin-4-yl)hexan-1-ol

To a stirred solution of 6-[1-(2-bromopyridin-4-yl)piperidin-4-yl]hexan-1-ol (500 mg, 2 mmol, Intermediate PX) and (3aR,7aR)-4-(3-fluorophenyl)-octahydropyrrolo[3,2-b]pyridine hydrochloride (376.14 mg, 1.465 mmol, Intermediate PT) in 1,4-dioxane (5 mL) were added {1,3-bis[2,6-bis(pentan-3-yl)phenyl]-4,5-dichloro-2,3-dihydro-1H-imidazol-2-yl}dichloro(2-methyl-lanbda4-pyridin-1-yl)palladium (123.23 mg, 0.147 mmol) and Cs₂CO₃ (1431.99 mg, 4.395 mmol) in portions at rt under nitrogen atmosphere. The resulting mixture was stirred for 16 h at 80° C. under nitrogen atmosphere. On completion, the mixture was purified by silica gel column chromatography, eluted with CH₂Cl₂/MeOH (10:1), to afford the title (277 mg, 39% yield) as a yellow oil. ¹H NMR (400 MHz, Chloroform-d) δ 13.23 (s, 1H), 7.78 (s, 1H), 7.26-7.17 (m, 1H), 6.75-6.66 (m, 1H), 6.64-6.57 (m, 1H), 6.56-6.50 (m, 1H), 6.32-6.26 (m, 1H), 5.54 (s, 1H), 5.32 (s, OH), 4.55-4.45 (m, 1H), 4.40-4.19 (m, 1H), 4.07-3.85 (m, 3H), 3.68 (t, J=6.6 Hz, 2H), 3.51 (s, 3H), 3.04 (t, J=12.7 Hz, 2H), 2.94-2.85 (in. 1H), 2.35 (d, J=12.2 Hz, 1H), 2.26-2.03 (m, 2H), 1.87 (d, J=10.5 Hz, 3H), 1.80-1.54 (in, 3H), 1.45-1.18 (m, 11H). 19F NMR (377 MHz, CDCl₃) δ −74.96, −75.11, −75.60, −112.03. LC/MS (ESI, m/z): [(M+1)]⁺=481.25.

### Step 2—6-(1-{2-[(3aR,7aR)-4-(3-fluorophenyl)-hexahydro-2H-pyrrolo[3,2-b]pyridin-1-yl]pyridin-4-yl}piperidin-4-yl)hexanal

To a stirred solution of 6-(1-{2-[(3aR,7aR)-4-(3-fluorophenyl)-hexahydro-2H-pyrrolo[3,2-b]pyridin-1-yl]pyridin-4-yl}piperidin-4-yl)hexan-1-ol (277 mg, 0.576 mmol) in DCM (3 mL) was added Dess-Martin periodinane (733.27 mg, 1.728 mmol) in portions at rt under nitrogen atmosphere. The resulting mixture was stirred for 16 h at rt under nitrogen atmosphere. On completion, the mixture was concentrated under vacuum. The residue was dissolved in DCM (3 mL), then the residue was purified by Prep-TLC (CH₂Cl₂/MeOH 8:1) to afford the title compound (95 mg, 34% yield) as a yellow oil. LC/MS (ESI, m/z): [(M+1)]⁺=479.5.

### 4-(4-{[2-(Benzyloxy)ethoxy]-methyl}piperidin-1-yl)-2-bromopyridine (Intermediate PZ)

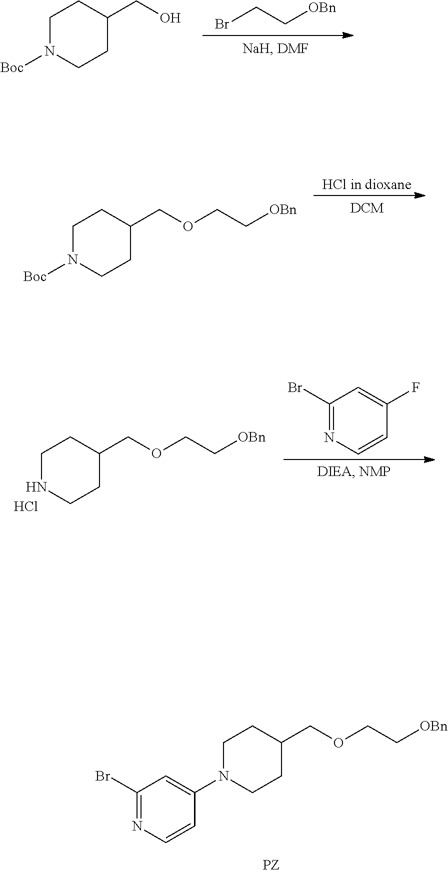

### Step 1—Tert-butyl 4-{[2-(benzyloxy)ethoxy]methyl}piperidine-1-carboxylate

To a stirred solution of tert-butyl 4-(hydroxymethyl)piperidine-1-carboxylate (4.5 g, 21 mmol) in DMF (45 mL) was added NaH (1.25 g, 31.4 mmol, 60% dispersion in mineral oil) at 0° C. under nitrogen atmosphere. The resulting mixture was stirred for 30 min at rt under nitrogen atmosphere. To the above mixture was added [(2-bromoethoxy)methyl]benzene (4.95 g, 23.0 mmol) dropwise at 0° C. The resulting mixture was stirred for an additional 16 h at rt. On completion, the reaction was quenched with sat. NH₄Cl (aq.) at 0° C. The residue was dissolved in water (120 mL). The aqueous layer was extracted with EtOAc (3×30 mL). The organic layer was concentrated under reduced pressure. The residue was purified by reverse-phase flash chromatography (column, C18 silica gel; mobile phase, MeCN in Water (0.1% TFA), 50% to 70% gradient in 10 min; detector, UV 254 nm; desired fractions were collected at 63% B and concentrated under reduced pressure) to afford the title compound (2.1 g, 29% yield) as a yellow oil. ¹H NMR (400 MHz, Chloroform-d) δ 7.41-7.29 (m, 5H), 4.60 (s, 2H), 4.12 (d, J=13.3 Hz, 2H), 3.67-3.60 (m, 4H), 3.34 (d, J=6.3 Hz, 2H), 2.78-2.66 (m, 2H), 1.85-1.70 (m, 3H), 1.48 (s, 9H), 1.23-1.08 (m, 2H); LC/MS (ESI, m/z): [(M+H−56)]⁺=294.1.

### Step 2—4-{[2-(Benzyloxy)ethoxy]methyl}piperidine hydrochloride

To a stirred solution of tert-butyl 4-{[2-(benzyloxy)ethoxy]methyl}piperidine-1-carboxylate (2.1 g. 6.0 mmol) in DCM (15 mL) was added 4 M HCl (gas) in 1,4-dioxane (15 mL, 60 mmol) dropwise at rt under air atmosphere. The resulting mixture was stirred for 1 h at rt under air atmosphere. On completion, the mixture was concentrated under reduced pressure. The residue was purified by trituration with Et₂O (30 mL) and dried under reduced pressure to afford the title compound (1.8 g) as a white solid. ¹H NMR (400 MHz, Chloroform-d) δ 9.50 (d, J=132.0 Hz, 1H), 7.41-7.27 (m, 5H), 4.57 (s, 2H), 3.62 (s. 4H), 3.50 (d, J=12.6 Hz, 2H), 3.37 (d, J=6.5 Hz, 2H), 2.93-2.80 (m, 2H), 2.03-1.95 (in, 2H), 1.95-1.82 (m, 1H), 1.74-1.59 (m, 2H); LC/MS (ESI, m/z): [(M+H)]⁺=250.2.

### Step 3—4-(4-{[2-(Benzyloxy)ethoxy]methyl}piperidin-1-yl)-2-bromopyridine

To a stirred solution of 4-{[2-(benzyloxy)ethoxy]methyl}piperidine hydrochloride (1.8 g, 6.3 mmol) in NMP (15 mL) was added 2-bromo-4-fluoropyridine (1.33 g, 7.56 mmol) at rt under air atmosphere. The resulting mixture was stirred for 1 h at 130° C. under air atmosphere. On completion, the mixture was cooled to rt and concentrated under reduced pressure. The residue was purified by reverse-phase flash chromatography (column, C18 silica gel; mobile phase, MeCN in Water (10 mmol/L NH₄HCO₃), 40% to 70% gradient in 30 min; detector, UV 254 n; desired fractions were collected at 67% B and concentrated under reduced pressure) to afford as a title compound (2.1 g, 82% yield) as a brown yellow oil. ¹H NMR (400 MHz, Chloroform-d) δ 7.97 (d, J=6.0 Hz, 1H), 7.36 (d, J=4.4 Hz, 4H), 7.34-7.26 (m, 1H), 6.81 (d, J=2.4 Hz, 1H), 6.63-6.56 (m, 1H), 4.59 (s, 2H), 3.89-3.79 (m, 2H), 3.64 (s, 4H), 3.36 (d, J=6.2 Hz, 2H), 2.95-2.84 (m, 2H), 1.98-1.81 (m, 3H), 1.38-1.23 (m, 2H): LC/MS (ESI, m/z): [(M+H)]⁺=405.2: LC/MS (ESI, m/z): [(M+H+2)]*=407.2.

### 2-[(3aR,7aR)-4-(3-fluorophenyl)-hexahydro-2H-pyrrolo[3,2-b]pyridin-1-yl]-4-{4-[(2-iodoethoxy)methyl]piperidin-1-yl}pyridine (Intermediate OA)

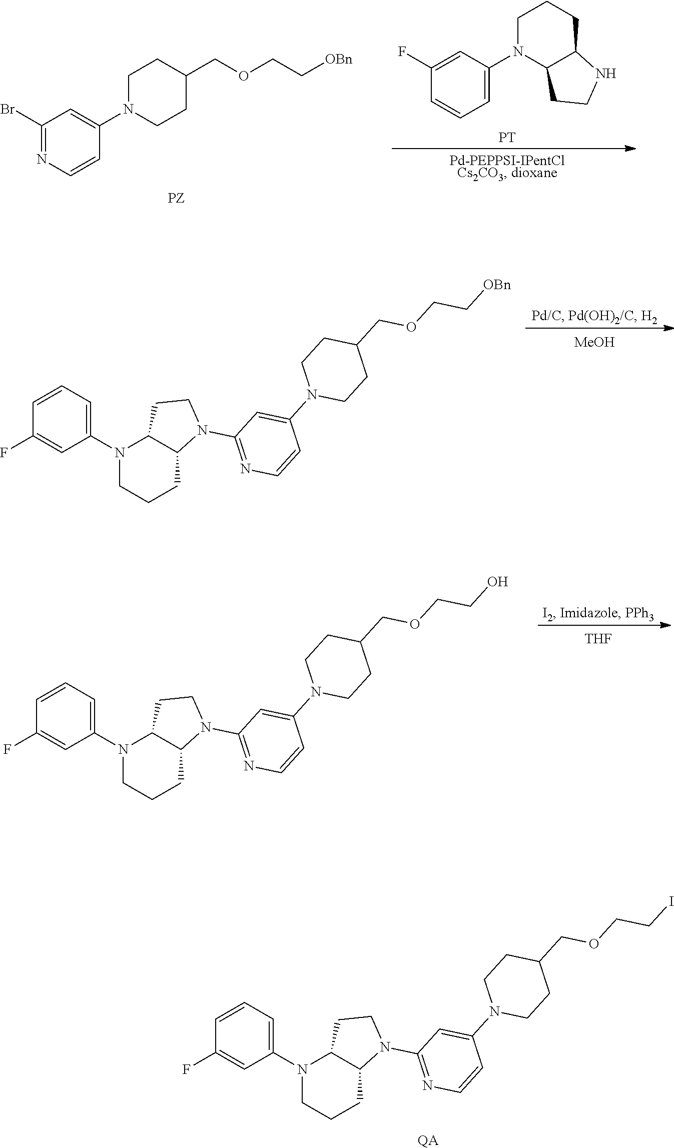

### Step 1—2-[(3aR,7aR)-4-(3-fluorophenyl)-hexahydro-2H-pyrrolo[3,2-b]pyridin-1-yl]-4-(4-{[2-(benzyloxy)ethoxy]methyl}piperidin-1-yl)pyridine

To a stirred solution of 4-(4-{[2-(benzyloxy)ethoxy]methyl}piperidin-1-yl)-2-bromopyridine (1.10 g, 2.72 mmol, Intermediate PZ) and (3aR,7aR)-4-(3-fluorophenyl)-octahydropyrrolo[3,2-b]pyridine (500 mg, 2.27 mmol, Intermediate PT) in 1,4-dioxane (10 mL) were added Cs₂CO₃ (2.22 g, 6.81 mmol) and Pd-PEPPSI-IPentCl 2-methylpyridine (286.38 mg, 0.340 mmol) at rt under air atmosphere. The resulting mixture was stirred for 16 h at 100° C. under nitrogen atmosphere. On completion, the mixture was cooled to rt and concentrated under reduced pressure. The residue was purified by reverse-phase flash chromatography (column. C18 silica gel; mobile phase, MeCN in Water (0.1% TFA), 30% to 60% gradient in 30 min; detector, UV 254 nm; desired fractions were collected at 52% B and concentrated under reduced pressure) to afford the title compound (720 mg, 58% yield) as a brown yellow oil. ¹H NMR (400 MHz, Chloroform-d) δ 7.76-7.69 (m, 1H), 7.39-7.33 (m, 5H), 7.27-7.17 (m, 1H), 6.73-6.66 (m, 1H), 6.65-6.58 (m, 1H), 6.58-6.49 (m, 1H), 6.33-6.26 (m, 1H), 5.57-5.52 (m, 1H), 4.59 (s, 2H), 4.55-4.44 (m, 1H), 4.23 (s, 1H), 3.95 (d, J=13.4 Hz, 2H), 3.65 (s, 4H), 3.50 (t, J=10.0 Hz, 2H), 3.39 (d, J=6.3 Hz, 2H), 3.10-3.02 (m, 2H), 2.96-2.85 (m, 1H), 2.33 (d, J=12.5 Hz, 1H), 2.11-1.91 (m, 6H), 1.78-1.64 (m, 1H), 1.42-1.26 (in, 4H); LC/MS (ESI, m/z): [(M+H)]⁺=545.25.

### Step 2—2-[(1-{2-[(3aR,7aR)-4-(3-fluorophenyl)-hexahydro-2H-pyrrolo[3,2-b]pyridin-1-yl]pyridin-4-yl}piperidin-4-yl)methoxy]ethanol

To a solution of 2-[(3aR,7aR)-4-(3-fluorophenyl)-hexahydro-2H-pyrrolo[3,2-b]pyridin-1-yl]-4-(4-{[2-(benzyloxy)ethoxy]methyl}piperidin-1-yl)pyridine (720 mg, 1.32 mmol) in 2 mL MeOH was added Pd/C (100 mg) and Pd(OH)₂/C (100 mg) in a pressure tank. The mixture was hydrogenated at rt under 30 psi of hydrogen pressure for 1 h. On completion, the mixture was filtered through a Celite pad and concentrated under reduced pressure to give the title compound (500 mg, 83% yield) as a brown yellow oil. ¹H NMR (400 MHz, Chloroform-d) S 13.50 (s, 1H), 7.83 (s, 1H), 7.27-7.16 (m, 1H), 6.73-6.65 (m, 1H), 6.65-6.57 (m, 1H), 6.57-6.49 (m, 1H), 6.29 (d, J=6.3 Hz, 1H), 5.55 (s, 1H), 4.55-4.44 (m, 1H), 4.28 (s, 1H), 4.01-3.93 (m, 2H), 3.78 (s, 1H), 3.70-3.63 (m, 1H), 3.59 (s, 2H), 3.41 (d, J=6.0 Hz, 2H), 3.14-3.03 (m, 2H), 2.95-2.85 (m, 1H), 2.40-2.32 (m, 1H), 2.26-2.04 (m, 3H), 1.98-1.85 (m, 3H), 1.77-1.69 (m, 1H), 1.44-1.26 (m, 4H); LC/MS (ESI, m/z): [(M+H)]⁺=455.2.

### Step 3—2-[(3aR,7aR)-4-(3-fluorophenyl)-hexahydro-2H-pyrrolo[3,2-b]pyridin-1-yl]-4-{4-[(2-iodoethoxy)methyl]piperidin-1-yl}pyridine

To a stirred solution of 2-[(1-{2-[(3aR,7aR)-4-(3-fluorophenyl)-hexahydro-2H-pyrrolo[3,2-b]pyridin-1-yl]pyridin-4-yl}piperidin-4-yl)methoxy]ethanol (100 mg, 0.22 mmol) in THF (2 mL) were added PPh₃ (144.25 mg, 0.550 mmol) and Imidazole (37.44 mg, 0.550 mmol) at rt under air atmosphere. To the above mixture was added I₂ (139.58 mg, 0.550 mmol) at rt and the mixture was stirred for 2 h at rt. On completion, the mixture was concentrated under reduced pressure. The residue was purified by Prep-TLC (CH₂Cl₂/MeOH 8:1) to afford the title compound (100 mg, 81% yield) as a brown oil. LC/MS (ESI, m/z): [(M+H)]⁺=565.2.

### Tert-butyl (3aR,7aR)-3-[2-(2-methoxyethoxy)ethyl]-octahydropyrrolo[3,2-b]pyridine-1-carboxylate (Intermediate OB)

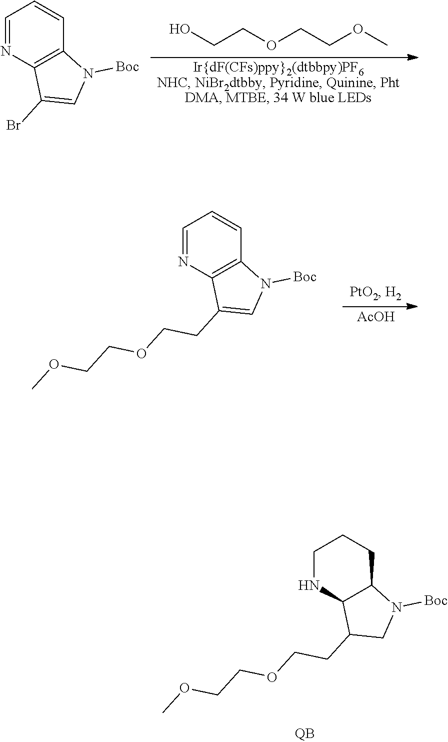

### Step 1—tert-butyl 3-[2-(2-methoxyethoxy)ethyl]pyrrolo[3,2-b]pyridine-1-carboxylate

To a stirred solution of diglycol methyl ether (1.82 g, 15.144 mmol) in 2-methoxy-2-methylpropane (10 mL) was added 5,7-di-tert-butyl-3-phenyl-1,3lambda5-benzoxazol-3-ylium tetrafluoroborate (5.99 g, 15.1 mmol) at rt under nitrogen atmosphere. The resulting mixture was stirred for 20 min at rt under nitrogen atmosphere. To the above mixture was added pyridine (0.80 g, 10 mmol) dropwise at rt and the mixture was stirred for additional 30 min at rt. Then the mixture was filtered, the filter cake was washed with TBME (1 mL), and the filtrate was collected. To the above solution were added tert-butyl 3-bromopyrrolo[3,2-b]pyridine-1-carboxylate (3 g, 10 mmol, CAS #192189-15-4), 8,8-dibromo-4,12-di-tert-butyl-7lambda4,9lambda4-diaza-8-nickelatricyclo[7.4.0.0\hat{ }{2,7}]trideca-1 (13), 2,4,6,9,11-hexaene (0.25 g, 0.51 mmol), 1-azabicyclo[2.2.2]octane (1.96 g, 17.7 mmol) and 2,3-dihydro-1H-isoindole-1,3-dione (0.30 g, 2.0 mmol) in DMA (10 mL) at rt. The vial was stirred at 1500 rpm stir rate and irradiated under 450 nm LED modules at 100% light intensity with maxed fan speed of 1500 rpm stirring rate in a PennOC Integrated Photoreactor for 2 hrs. On completion, the mixture was concentrated under reduced pressure. The residue was purified by reverse phase flash chromatography (Column: WelFlash™ C18-I, 20-40 um, 330 g: Eluent A: Water (plus 10 mmol/L NH₄HCO₃); Eluent B: ACN; Gradient: 30%-80% B in 30 min; Flow rate: 85 mL/min; Detector: 220 nm; desired fractions were collected at 80% B and concentrated under reduced pressure) to afford the title compound (2 g, 62% yield) as a light yellow oil. LC/MS (ESI, m/z): [(M+H)]⁺=321.2.

### Step 2—Tert-butyl (3aR,7aR)-3-[2-(2-methoxyethoxy)ethyl]-octahydropyrrolo[3,2-b]pyridine-1-carboxylate

To a solution of tert-butyl 3-[2-(2-methoxyethoxy)ethyl]pyrrolo[3,2-b]pyridine-1-carboxylate (2 g, 6 mmol) in AcOH (20 mL) was added PtO₂ (708.77 mg, 3.121 mmol) under nitrogen atmosphere. The reaction system was degassed under vacuum and purged with H₂ several times, then it was hydrogenated under H₂ balloon (˜1 atm) at rt for 4 h. After completion of the reaction, PtO₂ was filtered off through celite and the filter cake was washed with MeOH (3×50 mL). The corresponding filtrate was concentrated under reduced pressure. The residue was diluted with water (100 mL). The mixture was basified to pH 9 with saturated Na₂CO₃ (aq.). The resulting mixture was extracted with EtOAc (3×100 mL). The combined organic layers were washed with brine (2×100 mL), and dried over anhydrous Na₂SO₄. After filtration, the filtrate was concentrated under reduced pressure to afford the title compound (1.1 g, 54% yield) as a brown oil. LC/MS (ESI, m/z): [(M+H)]⁺=329.3.

### (3aR,7aR)-4-(3-fluorophenyl)-3-[2-(2-methoxyethoxy)ethyl]-octahydropyrrolo[3,2-b]pyridine hydrochloride (Intermediate QC)

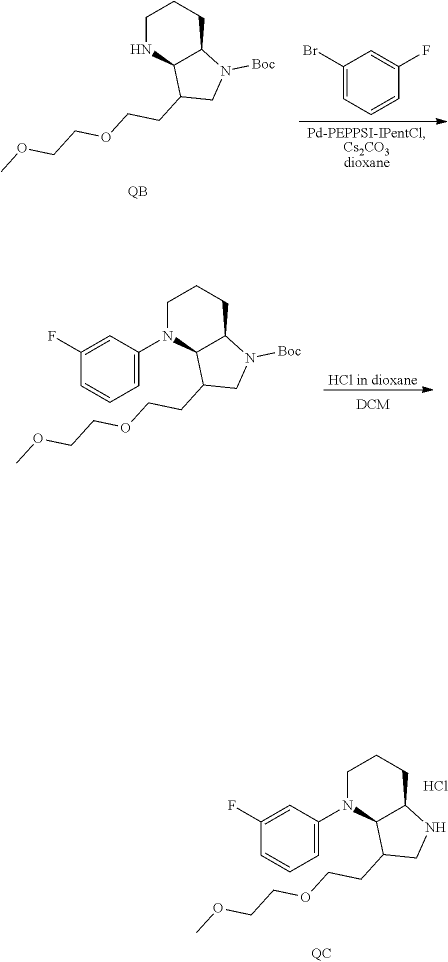

### Step 1—Tert-butyl (3aR,7aR)-4-(3-fluorophenyl)-3-[2-(2-methoxyethoxy)ethyl]-hexahydro-2H-pyrrolo[3,2-b]pyridine-1-carboxylate

To a stirred solution of tert-butyl (3aR,7aR)-3-[2-(2-methoxyethoxy)ethyl]-octahydropyrrolo[32-b]pyridine-1-carboxylate (1.1 g. 3.4 mmol, Intermediate QB) and M-bromofluorobenzene (0.88 g, 5.02 mmol) in dioxane (10 mL) were added Pd-PEPPSI-IPentCl 2-methylpyridine (o-picoline (0.28 g, 0.34 mmol) and Cs₂CO₃ (3.27 g, 10.1 mmol) at rt under nitrogen atmosphere. The resulting mixture was stirred for 16 h at 80° C. under nitrogen atmosphere. On completion, the mixture was cooled to rt and concentrated under reduced pressure. The residue was purified by reverse phase flash chromatography (Column: WelFlash™ C18-I, 20-40 um, 330 g: Eluent A: Water (plus 10 mmol/L NH4HCO₃): Eluent B: ACN; Gradient: 30%-70% B in 30 min; Flow rate: 85 mL/min; Detector: 220 nm; desired fractions were collected at 65% B and concentrated under reduced pressure) to afford the title compound (800 mg, 57% yield) as a light yellow oil. LC/MS (ESI, m/z): [(M+H)]=423.2.

### Step 2—(3aR,7aR)-4-(3-fluorophenyl)-3-[2-(2-methoxyethoxy)ethyl]-octahydropyrrolo[3,2-b]pyridine hydrochloride

To a stirred solution of tert-butyl (3aR,7aR)-4-(3-fluorophenyl)-3-[2-(2-methoxyethoxy)ethyl]-hexahydro-2H-pyrrolo[3,2-b]pyridine-1-carboxylate (800 mg, 1.89 mmol) in DCM (4 mL) was added HCl (gas) in 1,4-dioxane (4 mL) at rt under nitrogen atmosphere. The resulting mixture was stirred for 1 h at rt under nitrogen atmosphere. On completion, the mixture was concentrated under reduced pressure to afford the title compound (800 mg, 94% yield) as a light yellow oil. LC/MS (ESI, m/z): [(M+H)]⁻=323.2.

### 2-[(3R,3aS,7aS)-4-(3-fluorophenyl)-3-[2-(2-methoxyethoxy)ethyl]-hexahydro-2H-pyrrolo[3,2-b]pyridin-1-yl]-4-(piperidin-1-yl)pyridine (Intermediate QD) and 2-[(3S,3aS,7aS)-4-(3-fluorophenyl)-3-[2-(2-methoxyethoxy)ethyl]-hexahydro-2H-pyrrolo[3,2-b]pyridin-1-yl]-4-(piperidin-1-yl)pyridine (Intermediate OE) 2-[(3R,3aS,7aS)-4-(3-fluorophenyl)-3-[2-(2-methoxyethoxy)ethyl]-hexahydro-2H-pyrrolo[3,2-b]pyridin-1-yl]-4-(piperidin-1-yl)pyridine (Intermediate QF) and 2-[(3R,3aS,7aS)-4-(3-fluorophenyl)-3-[2-(2-methoxyethoxy)ethyl]-hexahydro-2H-pyrrolo[3,2-b]pyridin-1-yl]-4-(piperidin-1-yl)pyridine (Intermediate QG)

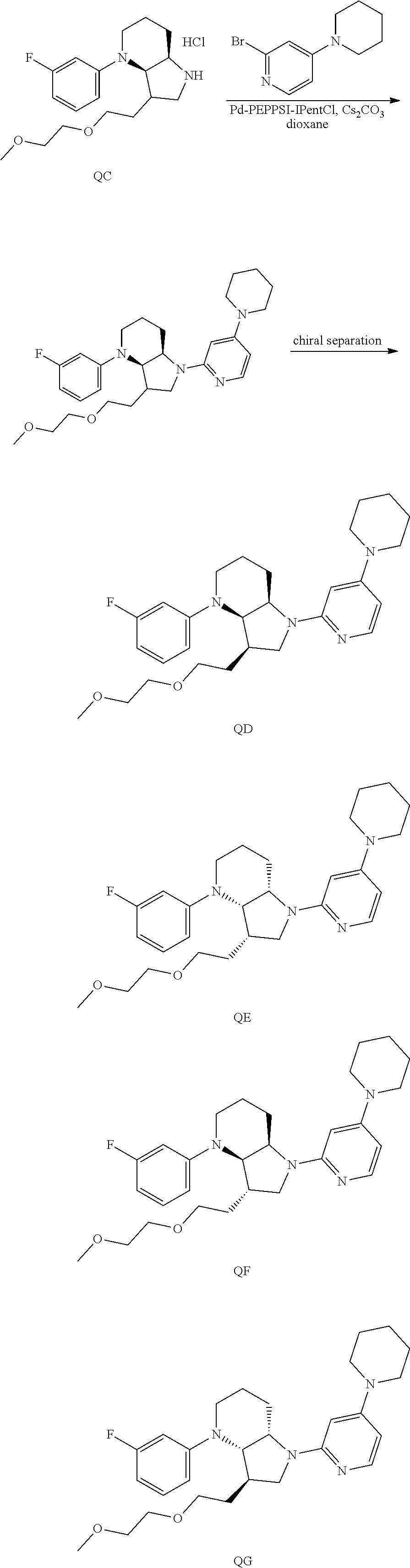

### Step 1—2-[(3aR,7aR)-4-(3-fluorophenyl)-3-[2-(2-methoxyethoxy)ethyl]-hexahydro-2H-pyrrolo[3,2-b]pyridin-1-yl]-4-(piperidin-1-yl)pyridine

To a stirred solution of (3aR,7aR)-4-(3-fluorophenyl)-3-[2-(2-methoxyethoxy)ethyl]-octahydropyrrolo[3,2-b]pyridine hydrochloride (800 mg, 2.23 mmol, Intermediate QC) and 2-bromo-4-(piperidin-1-yl)pyridine (645.03 mg, 2.675 mmol. CAS #24255-92-3) in dioxane (10 mL) were added Pd-PEPPSI-IPentCl 2-methylpyridine (o-picoline) (187.51 mg, 0.223 mmol) and Cs₂CO₃ (2.18 g. 6.69 mmol) at rt under nitrogen atmosphere. The resulting mixture was stirred for 16 h at 80° C. under nitrogen atmosphere. On completion, the mixture was cooled to rt and concentrated under reduced pressure. The residue was purified by reverse phase flash chromatography (Column: WelFlash™ C18-I, 20-40 um, 330 g; Eluent A: Water (plus 10 mmol/L NH₄HCO₃); Eluent B: ACN; Gradient: 30%-70% B in 30 min; Flow rate: 85 mL/min: Detector: 220 nm; desired fractions were collected at 58% B and concentrated under reduced pressure) to afford the title compound (600 mg, 56% yield) as a light yellow oil. LC/MS (ESI, m/z): [(M+H)]⁺=483.2.

### Step 2—2-[(3R,3aS,7aS)-4-(3-fluorophenyl)-3-[2-(2-methoxyethoxy)ethyl]-hexahydro-2H-pyrrolo[3,2-b]pyridin-1-yl]-4-(piperidin-1-yl)pyridine and 2-[(3S,3aS,7aS)-4-(3-fluorophenyl)-3-[2-(2-methoxyethoxy)ethyl]-hexahydro-2H-pyrrolo[3,2-b]pyridin-1-yl]-4-(piperidin-1-yl)pyridine, 2-[(3R,3aS,7aS)-4-(3-fluorophenyl)-3-[42-methoxyethoxy)ethyl]-hexahydro-2H-pyrrolo[3,2-b]pyridin-1-yl]-4-(piperidin-1-yl)pyridine

2-[(3aR,7aR)-4-(3-fluorophenyl)-3-[2-(2-methoxyethoxy)ethyl]-hexahydro-2H-pyrrolo[3,2-b]pyridin-1-yl]-4-(piperidin-1-yl)pyridine (1 g, 2.072 mmol) was separated by HPLC (Column: CHIRALPAK ID, 2*25 cm, 5 m; Mobile Phase A: HEX (0.5% 2M NH₃-MeOH), Mobile Phase B: Hex:DCM=1:1-HPLC; Flow rate: 20 mL/min; Gradient: isocratic 20; Wave Length: 220/254 nm; RT1 (min): 18.202; RT2 (min): 26.502; Sample Solvent: EtOH: Injection Volume: 1.0 mL: Number Of Runs: 10) to afford 2-[(3R,3aR,7aR)-4-(3-fluorophenyl)-3-[2-(2-methoxyethoxy)ethyl]-hexahydro-2H-pyrrolo[3,2-b]pyridin-1-yl]-4-(piperidin-1-yl)pyridine (100 mg, 10%) and 2-[(3S,3aS,7aS)-4-(3-fluorophenyl)-3-[2-(2-methoxyethoxy)ethyl]-hexahydro-2H-pyrrolo[3,2-b]pyridin-1-yl]-4-(piperidin-1-yl)pyridine (100 mg, 10%), 2-[(3S,3aS,7aS)-4-(3-fluorophenyl)-3-[2-(2-methoxyethoxy)ethyl]-hexahydro-2H-pyrrolo[3,2-b]pyridin-1-yl]-4-(piperidin-1-yl)pyridine (100 mg, 10%), 2-[(3R,3aS,7aS)-4-(3-fluorophenyl)-3-[2-(2-methoxyethoxy)ethyl]-hexahydro-2H-pyrrolo[3,2-b]pyridin-1-yl]-4-(piperidin-1-yl)pyridine (260 mg, 26%), and 2-[(3R,3aS,7aS)-4-(3-fluorophenyl)-3-[2-(2-methoxyethoxy)ethyl]-hexahydro-2H-pyrrolo[3,2-b]pyridin-1-yl]-4-(piperidin-1-yl)pyridine (70 mg, 7%), as a light yellow oils. LC/MS (ESI, m/z): [(M+H)]⁺=483.2 for all isomers. The absolute stereochemistry of the compounds were assigned arbitrarily.

### 2-[(3R,3aR,7aR)-4-(3-fluorophenyl)-3-[2-(2-iodoethoxy)ethyl]-hexahydro-2H-pyrrolo[3,2-b]pyridin-1-yl]-4-(piperidin-1-yl)pyridine (Intermediate OH)

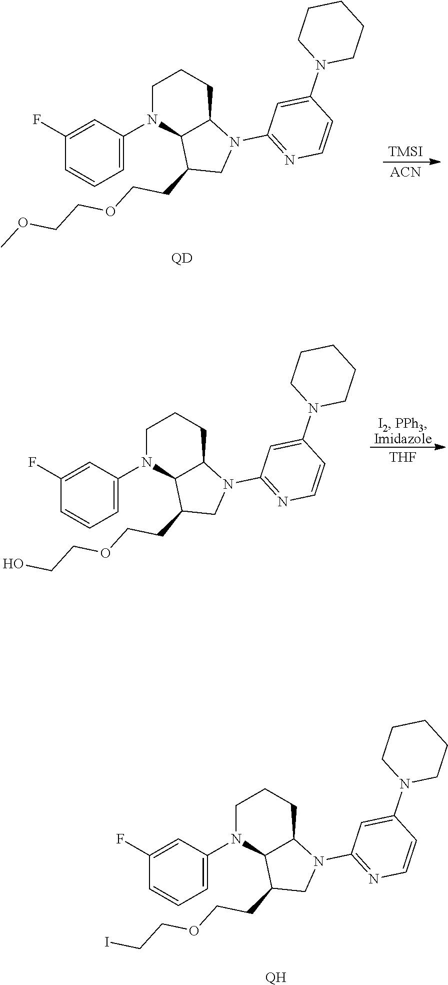

### Step 1—2-{2-[(3R,3aR,7aR)-4-(3-fluorophenyl)-1-[4-(piperidin-1-yl)pyridin-2-yl]-hexahydro-2H-pyrrolo[3,2-b]pyridin-3-yl]ethoxy}ethanol

To a stirred solution of 2-[(3R,3aR,7aR)-4-(3-fluorophenyl)-3-[2-(2-methoxyethoxy)ethyl]-hexahydro-2H-pyrrolo[3,2-b]pyridin-1-yl]-4-(piperidin-1-yl)pyridine (100 mg, 0.21 mmol, Intermediate QD) in ACN (4 mL) was added TMSI (82.92 mg, 0.414 mmol) at rt under nitrogen atmosphere. The resulting mixture was stirred for 16 h at rt under nitrogen atmosphere. On completion, the mixture was concentrated under reduced pressure. The residue was purified by reverse phase flash chromatography (Column: WelFlash™ C18-I, 20-40 um, 120 g; Eluent A: Water (plus 10 mmol/L NH₄HCO₃); Eluent B: MeOH; Gradient: 40%-95% B in 30 min; Flow rate: 85 mL/min; Detector: 220 nm; desired fractions were collected at 89% B and concentrated under reduced pressure) to afford the title compound (80 mg, 82% yield) as a light yellow oil. LC/MS (ESI, m/z): [(M+H)]⁺=469.2.

### Step 2—2-[(3R,3aR,7aR)-4-(3-fluorophenyl)-3-[2-(2-iodoethoxy)ethyl]-hexahydro-2H-pyrrolo[3,2-b]pyridin-1-yl]-4-(piperidin-1-yl)pyridine

To a stirred solution of 2-{2-[(3R,3aR,7aR)-4-(3-fluorophenyl)-1-[4-(piperidin-1-yl)pyridin-2-yl]-hexahydro-2H-pyrrolo[3,2-b]pyridin-3-yl]ethoxy}ethanol (80 mg, 0.17 mmol) and PPh₃ (67.17 mg, 0.257 mmol) in THF (2 mL) was added Imidazole (17.43 mg, 0.257 mmol) at rt under nitrogen atmosphere and the mixture was stirred for 0.5 h at rt. To the above mixture was added 12 (86.66 mg, 0.342 mmol) and the mixture was stirred for additional 2 h at rt. On completion, the mixture was concentrated under reduced pressure. The residue was purified by reverse phase flash chromatography (Column: WelFlash™ C18-I, 20-40 um, 120 g; Eluent A: Water (plus 10 mmol/L NH4HCO₃); Eluent B: MeOH; Gradient: 40%-95% B in 30 min; Flow rate: 85 mL/min: Detector: 220 nm; desired fractions were collected at 89% B and concentrated under reduced pressure) to afford the title compound (50 mg, 51% yield) as a light brown solid. LC/MS (ESI, m/z): [(M+H)]=579.2.

### 2-[(3S,3aS,7aS)-4-(3-fluorophenyl)-3-[2-(2-iodoethoxy)ethyl]-hexahydro-2H-pyrrolo[3,2-b]pyridin-1-yl]-4-(piperidin-1-yl)pyridine (Intermediate QI)

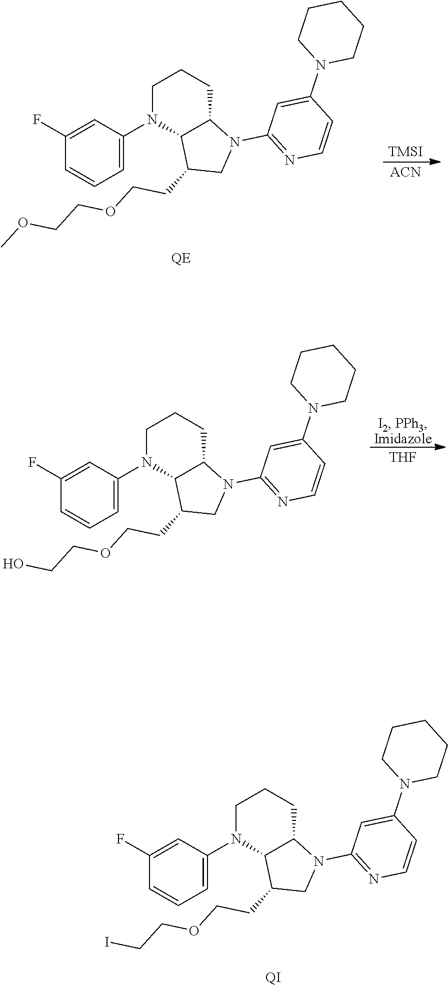

### Step 1—2-{2-[(3S,3aS,7aS)-4-(3-fluorophenyl)-1-[4-(piperidin-1-yl)pyridin-2-yl]-hexahydro-2H-pyrrolo[3,2-b]pyridin-3-yl]ethoxy}ethanol

To a stirred solution of 2-[(3S,3aS,7aS)-4-(3-fluorophenyl)-3-[2-(2-methoxyethoxy)ethyl]-hexahydro-2H-pyrrolo[3,2-b]pyridin-1-yl]-4-(piperidin-1-yl)pyridine (100 mg, 0.21 mmol, Intermediate QE) in ACN (2 mL) was added TMSI (124.37 mg, 0.621 mmol) at rt under nitrogen atmosphere. The resulting mixture was stirred for 2 h at rt under nitrogen atmosphere. On completion, the mixture was concentrated under reduced pressure. The residue was purified by reverse phase flash chromatography (Column: WelFlash™ C18-I, 20-40 um, 120 g; Eluent A: Water (plus 10 mmol/L NH4HCO₃): Eluent B: MeOH; Gradient: 40%-95% B in 30 min: Flow rate: 85 mL/min; Detector: 220 nm; desired fractions were collected at 89% B and concentrated under reduced pressure) to afford the title compound (90 mg, 93% yield) as a light yellow solid. LC/MS (ESI, m/z): [(M+H)]⁺=469.2.

### Step 2—2-[(3S,3aS,7aS)-4-(3-fluorophenyl)-3-[2-(2-iodoethoxy)ethyl]-hexahydro-2H-pyrrolo[3,2-b]pyridin-1-yl]-4-(piperidin-1-yl)pyridine

To a stirred solution of 2-{2-[(3S,3aS,7aS)-4-(3-fluorophenyl)-1-[4-(piperidin-1-yl)pyridin-2-yl]-hexahydro-2H-pyrrolo[3,2-b]pyridin-3-yl]ethoxy}ethanol (90 mg, 0.19 mmol) and PPh₃ (75.56 mg, 0.288 mmol) in THF (2 mL) was added Imidazole (19.61 mg, 0.288 mmol) at rt under nitrogen atmosphere. The resulting mixture was stirred for 0.5 h at rt under nitrogen atmosphere. To the above mixture was added I₂ (97.49 mg, 0.384 mmol) and the mixture was stirred for additional 2 h at rt. On completion, the mixture was concentrated under reduced pressure. The residue was purified by reverse phase flash chromatography (Column: WelFlash™ C18-I, 20-40 um, 120 g: Eluent A: Water (plus 10 mmol/L NH₄HCO₃); Eluent B: MeOH: Gradient: 40%-95% B in 30 min; Flow rate: 85 mL/min: Detector: 220 nm; desired fractions were collected at 89% B and concentrated under reduced pressure) to afford the title compound (80 mg, 72% yield) as a light brown solid. LC/MS (ESI, m/z): [(M+H)]⁺=579.2.

### 2-[(3S,3aR,7aR)-4-(3-fluorophenyl)-3-[2-(2-iodoethoxy)ethyl]-hcxahydro-2H-pyrrolo[3,2-b]pyridin-1-yl]-4-(piperidin-1-yl)pyridine (Intermediate QJ)

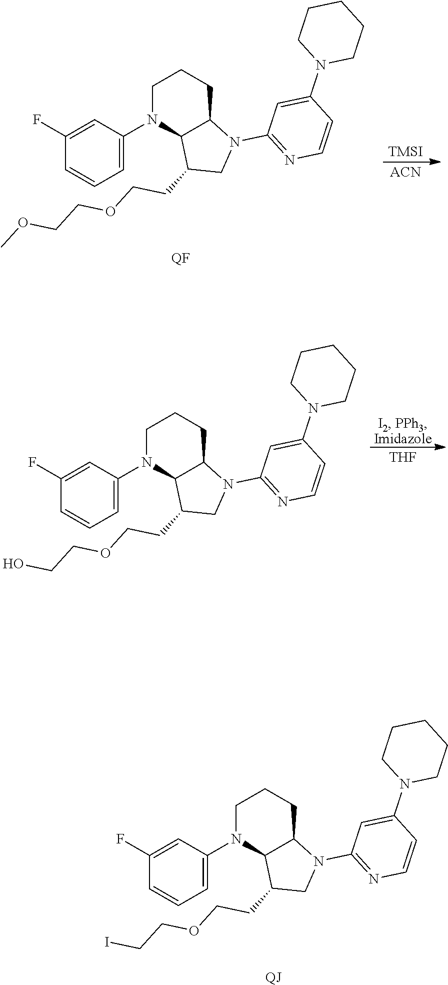

### Step 1—2-{2-[(3S,3aR,7aR)-4-(3-fluorophenyl)-1-[4-(piperidin-1-yl)pyridin-2-yl]-hexahydro-2H-pyrrolo[3,2-b]pyridin-3-yl]ethoxy}ethanol

To a stirred solution of 2-[(3S3aR,7aR)-4-(3-fluorophenyl)-3-[2-(2-methoxyethoxy)ethyl]-hexahydro-2H-pyrrolo[3,2-b]pyridin-1-yl]-4-(piperidin-1-yl)pyridine (100 mg, 0.21 mmol, Intermediate QF) in ACN (2 mL) was added TMSI (82.92 mg, 0.414 mmol) at rt under nitrogen atmosphere. The resulting mixture was stirred for 2 h at rt under nitrogen atmosphere. On completion, the mixture was concentrated under reduced pressure. The residue was purified by reverse phase flash chromatography (Column: WelFlash™ C18-I, 20-40 um, 120 g; Eluent A: Water (plus 10 mmol/L NH₄HCO₃); Eluent B: MeOH; Gradient: 40%-95% B in 30 min; Flow rate: 85 mL/min; Detector: 220 nm: desired fractions were collected at 89% B and concentrated under reduced pressure) to afford the title compound (80 mg, 82% yield) as a light yellow solid. LC/MS (ESI, m/z): [(M+H)]⁺=469.2.

### Step 2—2-[(3S,3aR,7aR)-4-(3-fluorophenyl)-3-[2-(2-iodoethoxy)ethyl]-hexahydro-2H-pyrrolo[3,2-b]pyridin-1-yl]-4-(piperidin-1-yl)pyridine

To a stirred solution of 2-{2-[(3S,3aR,7aR)-4-(3-fluorophenyl)-1-[4-(piperidin-1-yl)pyridin-2-yl]-hexahydro-2H-pyrrolo[3,2-b]pyridin-3-yl]ethoxy}ethanol (80 mg, 0.17 mmol) and PPh₃ (67.17 mg, 0.257 mmol) in THF (2 mL) was added Imidazole (17.43 mg, 0.257 mmol) at rt under nitrogen atmosphere and the mixture was stirred for 0.5 h at rt under nitrogen atmosphere. To the above mixture was added I₂ (86.66 mg, 0.342 mmol) and the resulting mixture was stirred for additional 2 h at rt. On completion, the mixture was concentrated under reduced pressure. The residue was purified by reverse phase flash chromatography (Column: WelFlash™ C18-I, 20-40 um, 120 g; Eluent A: Water (plus 10 mmol/L NH₄HCO₃); Eluent B: MeOH; Gradient: 40%-95% B in 30 min; Flow rate: 85 mL/min; Detector: 220 nm; desired fractions were collected at 89% B and concentrated under reduced pressure) to afford the title compound (60 mg, 61% yield) as a light yellow solid. LC/MS (ESI, m/z): [(M+H)]⁺=579.2.

### 2-[(3R,3aS,7aS)-4-(3-fluorophenyl)-3-[2-(2-iodoethoxy)ethyl]-hexahydro-2H-pyrrolo[3,2-b]pyridin-1-yl]-4-(piperidin-1-yl)pyridine (Intermediate QK)

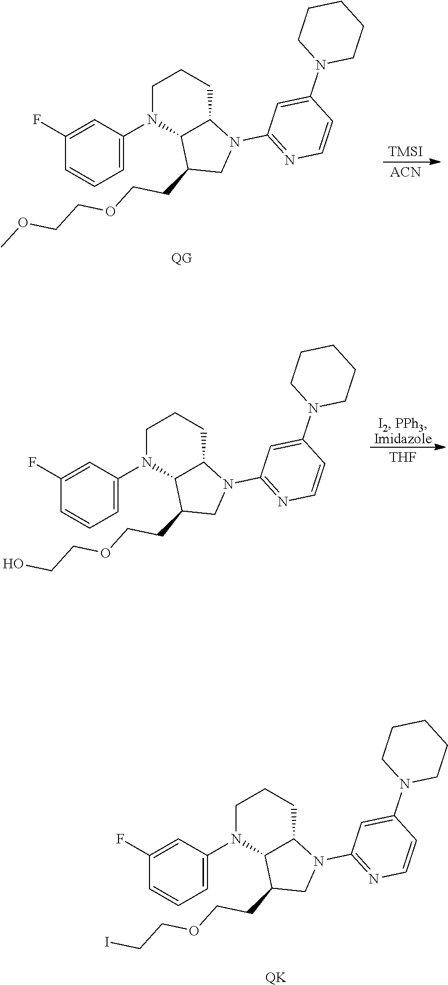

### Step 1—2-{2-[(3R,3aS,7aS)-4-(3-fluorophenyl)-1-[4-(piperidin-1-yl)pyridin-2-yl]-hexahydro-2H-pyrrolo[3,2-b]pyridin-3-yl]ethoxy}ethanol

To a stirred solution of 2-[(3R,3aS,7aS)-4-(3-fluorophenyl)-3-[2-(2-methoxyethoxy)ethyl]-hexahydro-2H-pyrrolo[3,2-b]pyridin-1-yl]-4-(piperidin-1-yl)pyridine (100 mg, 0.21 mmol, Intermediate QG) in ACN (2 mL) was added TMSI (82.92 mg, 0.414 mmol) at rt under nitrogen atmosphere. The resulting mixture was stirred for 2 h at rt under nitrogen atmosphere. On completion, the mixture was concentrated under reduced pressure. The residue was purified by reverse phase flash chromatography (Column: WelFlash™ C18-I, 20-40 um, 120 g; Eluent A: Water (plus 10 mmol/L NH₄HCO₃); Eluent B: MeOH; Gradient: 40%-95% B in 30 min; Flow rate: 85 mL/min; Detector: 220 nm: desired fractions were collected at 89% B and concentrated under reduced pressure) to afford the title compound (80 mg, 82% yield) as a light yellow solid. LC/MS (ESI, m/z): [(M+H)]⁺=469.2.

### Step 2—2-[(3R,3aS,7aS)-4-(3-fluorophenyl)-3-[2-(2-iodoethoxy)ethyl]-hexahydro-2H-pyrrolo[3,2-b]pyridin-1-yl]-4-(piperidin-1-yl)pyridine

To a stirred solution of 2-{2-[(3R,3aS,7aS)-4-(3-fluorophenyl)-1-[4-(piperidin-1-yl)pyridin-2-yl]-hexahydro-2H-pyrrolo[3,2-b]pyridin-3-yl]ethoxy}ethanol (80 mg, 0.17 mmol) and PPh₃ (67.17 mg, 0.257 mmol) in THF (2 mL) was added Imidazole (17.43 mg, 0.257 mmol) at rt under nitrogen atmosphere and the mixture was stirred for 0.5 h at rt under nitrogen atmosphere. To the above mixture was added I₂ (86.66 mg, 0.342 mmol) at and the resulting mixture was stirred for additional 2 h at rt. On completion, the mixture was concentrated under reduced pressure. The residue was purified by reverse phase flash chromatography (Column: WelFlash™ C18-I, 20-40 um, 120 g: Eluent A: Water (plus 10 mmol/L NH₄HCO₃); Eluent B: MeOH: Gradient: 40%-95% B in 30 min; Flow rate: 85 mL/min: Detector: 220 nm; desired fractions were collected at 89% B and concentrated under reduced pressure) to afford the title compound (60 mg, 61% yield) as a light yellow solid. LC/MS (ESI, m/z): [(M+H)]⁺=579.2.

### Tert-butyl 3-{2-[2-(2-methoxyethoxy)ethoxy]ethyl}-octahydropyrrolo[3,2-b]pyridine-1-carboxylate (Intermediate OL)

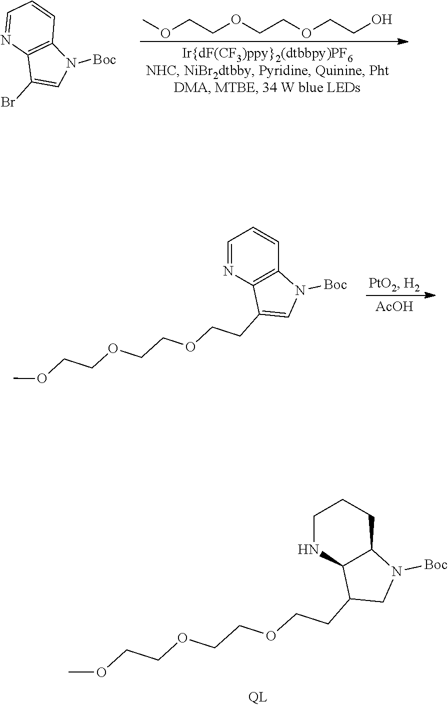 [SMILES: CC(C)(C)OC(=O)n1cc(Br)c2ncccc21 ; "AcOH" ; COCCOCCOCCO ; COCCOCCOCCc1cn(C(=O)OC(C)(C)C)c2cccnc12 ; COCCOCCOCCC1CN(C(=O)OC(C)(C)C)[C@@H]2CCCN[C@H]12]

### Step 1—Tert-butyl 3-{2-[2-(2-methoxyethoxy)ethoxy]ethyl}pyrrolo[3,2-b]pyridine-1-carboxylate

To a stirred mixture of tert-butyl 3-bromopyrrolo[3,2-b]pyridine-1-carboxylate (4 g, 10 mmol) and 5,7-di-tert-butyl-3-phenyl-1,3lambda5-benzoxazol-3-ylium (6.23 g, 20.2 mmol) in MTBE (10 mL) were added pyridine (1.06 g, 13.5 mmol) in portions at rt under air atmosphere and the mixture was stirred for 30 min at rt under air atmosphere. The resulting mixture was filtered, the filter cake was washed with MTBE (3×3 mL). To the above mixture was added methoxy triethylene glycol (3.32 g, 20.2 mmol), pyridine (1.06 g, 13.5 mmol) IR[DF(CF₃)PPY]₂(DTBPY)PF₆ (0.76 g, 0.67 mmol), 1-azabicyclo[2.2.2]octane (2.25 g, 20.19 mmol), 2,3-dihydro-1H-isoindole-1,3-dione (0.40 g, 2.692 mmol), 4,4′-Bis(tert-butyl)-2,2′-bipyridinenickel dibromide (0.33 g, 0.67 mmol) in portions over 30 min at rt. The vial was stirred at 1500 rpm stir rate and irradiated under 450 nm LED modules at 100% light intensity with maxed fan speed of 1500 rpm stirring rate in a PennOC Integrated Photoreactor for 2 hrs. On completion, the mixture was concentrated under reduced pressure. The residue was purified by reverse-phase flash chromatography (column, C18 silica gel; mobile phase, MeCN in Water (10 mmol/L NH4HCO3), 30% to 70% gradient in 40 min; detector, UV 254 nm; the resulting mixture was concentrated under reduced pressure) to afford the title compound (2.5 mg) as a yellow oil. LC/MS (ESI, m/z): [(M+1)]⁺=364.4.

### Step 2—Tert-butyl 3-{2-[2-(2-methoxyethoxy)ethoxy]ethyl}-octahydropyrrolo[3,2-b]pyridine-1-carboxylate

To a solution of tert-butyl 3-{2-[2-(2-methoxyethoxy)ethoxy]ethyl}pyrrolo[3,2-b]pyridine-1-carboxylate (3.1 g, 8.5 mmol) in 50 mL AcOH was added PtO₂ (20%, lg) under nitrogen atmosphere in a 250 mL round-bottom flask. The mixture was hydrogenated at rt for 16 h under hydrogen atmosphere using a hydrogen balloon. On completion, the mixture was filtered through a Celite pad and concentrated under reduced pressure. The residue was basified to pH 10 with saturated Na₂CO₃ (aq.). The aqueous layer was extracted with EtOAc (3×10 mL). The resulting mixture was concentrated under reduced pressure to give the title compound (2 g, 63% yield) as a yellow oil. LC/MS (ESI, m/z): [(M+1)]⁺=372.5.

### (3aR,7aR)-4-(3-fluorophenyl)-3-{2-[2-(2-methoxyethoxy)ethoxy]ethyl}-octahydropyrrolo[3,2-b]pyridine (Intermediate QM)

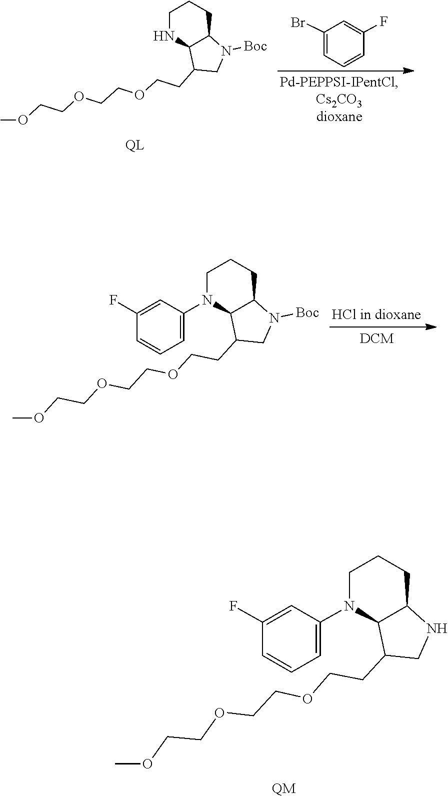

### Step 1—Tert-butyl (3aR,7aR)-4-(3-fluorophenyl)-3-{2-[2-(2-methoxyethoxy)ethoxy]ethyl}-hexahydro-2H-pyrrolo[3,2-b]pyridine-1-carboxylate

To a stirred mixture of tert-butyl 3-{2-[2-(2-methoxyethoxy)ethoxy]ethyl}-octahydropyrrolo[3,2-b]pyridine-1-carboxylate (1 g, 3 mmol, Intermediate QL) and M-bromofluorobenzene (0.70 g, 4.03 mmol) in 1,4-dioxane (12 mL) were added Cs₂CO₃ (1.75 g. 5.37 mmol) and Pd-PEPPSI-IPentCl 2-methylpyridine (o-picoline) (225.81 mg, 0.269 mmol) in portions at rt under air atmosphere. The resulting mixture was stirred for 16 h at 100° C. under nitrogen atmosphere. On completion, the mixture was cooled to rt and was purified by silica gel column chromatography, eluted with CH₂Cl₂/MeOH (12:1), to afford the title compound (800 mg, 64% yield) as a yellow solid. LC/MS (ESI, m/z): [(M+1)]*=466.6.

### Step 2—(3aR,7aR)-4-(3-fluorophenyl)-3-{2-[2-(2-methoxyethoxy)ethoxy]ethyl}-octahydropyrrolo[3,2-b]pyridine

To a stirred solution of tert-butyl (3aR,7aR)-4-(3-fluorophenyl)-3-{2-[2-(2-methoxyethoxy)ethoxy]ethyl}-hexahydro-2H-pyrrolo[3,2-b]pyridine-1-carboxylate (800 mg, 1.72 mmol) in DCM (10 mL) was added HCl (gas) in 1,4-dioxane (10 mL) dropwise at rt under air atmosphere and the mixture was stirred for 1 h at rt under air atmosphere. On completion, the mixture was concentrated under reduced pressure to give the title compound (800 mg) as a brown solid. LC/MS (ESI, m/z): [(M+1)]⁺=366.5.

(3R,3aR,7aR)-4-(3-fluorophenyl)-3-(2-(2-(2-methoxyethoxy)ethoxy)ethyl)-1-(4-(piperidin-1-yl)pyridin-2-yl)octahydro-1H-pyrrolo[3,2-b]pyridine (Intermediate ON) and (3S,3aS,7aS)-4-(3-fluorophenyl)-3-(2-(2-(2-methoxyethoxy)ethoxy)ethyl)-1-(4-(piperidin-1-yl)pyridin-2-yl)octahydro-1H-pyrrolo[3,2-b]pyridine (Intermediate QO)

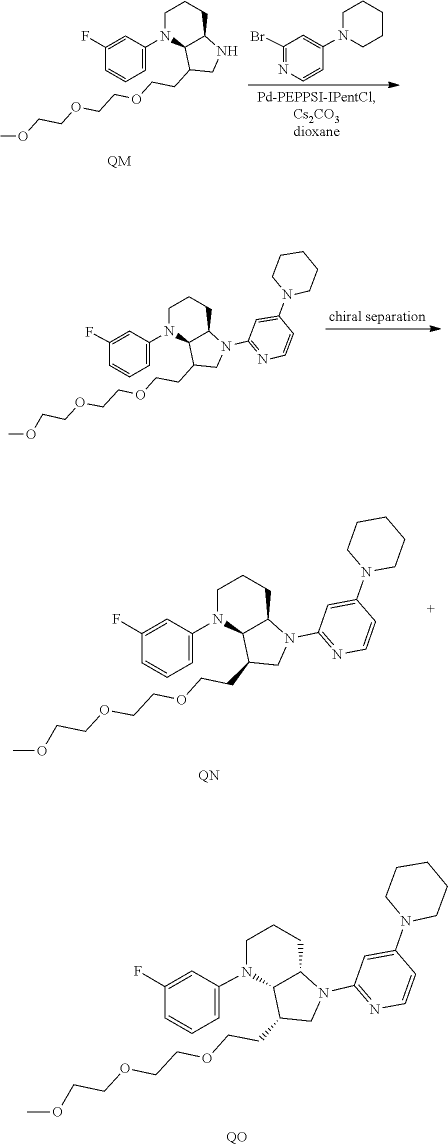

### Step 1—2-[(3aR,7aR)-4-(3-fluorophenyl)-3-{2-[2-(2-methoxyethoxy)ethoxy]ethyl}-hexahydro-2H-pyrrolo[3,2-b]pyridin-1-yl]-4-(piperidin-1-yl)pyridine

To a stirred mixture of (3aR,7aR)-4-(3-fluorophenyl)-3-{2-[2-(2-methoxyethoxy)ethoxy]ethyl}-octahydropyrrolo[3,2-b]pyridine (800 mg, 2.18 mmol, Intermediate QM) and 2-bromo-4-(piperidin-1-yl)pyridine (631.65 mg, 2.620 mmol) in 1,4-dioxane (10 mL) were added Pd-PEPPSI-IPentCl 2-methylpyridine (o-picoline) (183.62 mg, 0.218 mmol) and Cs₂CO₃ (2.13 g, 6.55 mmol) in portions at rt under air atmosphere. The resulting mixture was stirred for 16 h at 100° C. under nitrogen atmosphere. On completion, the mixture was cooled to rt and purified by silica gel column chromatography, eluted with CH₂Cl₂/MeOH (10:1), to afford the title compound (400 mg, 35% yield) as a brown oil. LC/MS (ESI, m/z): [(M+1)]⁺=526.7.

### Step 2—(3R,3aR,7aR)-4-(3-fluorophenyl)-3-(2-(2-(2-methoxyethoxy)ethoxy)ethyl)-1-(4-(piperidin-1-yl)pyridin-2-yl)octahydro-1H-pyrrolo[3,2-b]pyridine and (3S,3aS,7aS)-4-(3-fluorophenyl)-3-(2-(2-(2-methoxyethoxy)ethoxy)ethyl)-1-(4-(piperidin-1-yl)pyridin-2-yl)octahydro-1H-pyrrolo[3,2-b]pyridine

4-(3-Fluorophenyl)-3-(2-(2-methoxyethoxy)ethyl)-1-(4-(piperidin-1-yl)pyridin-2-yl)octahydro-1H-pyrrolo[3,2-b]pyridine (1 g, 2 mmol) was separated by SFC (CHIRALPAK ID, 2*25 cm, 5 μm; Mobile Phase A: Hex (0.5% 2M NH₃-MeOH)-HPLC, Mobile Phase B: EtOH:DCM=1:1-HPLC; Flow rate: 20 mL/min; Gradient: isocratic 30; Wave Length: 220/254 nm; RT1 (min): 7.083; RT2 (min): 10.31; Sample Solvent: MeOH:DCM=1:1 HPLC; Injection Volume: 0.5 mL; Number Of Runs: 20) to afford the first eluting isomer (3R,3aR,7aR)-4-(3-fluorophenyl)-3-(2-(2-(2-methoxyethoxy)ethoxy)ethyl)-1-(4-(piperidin-1-yl)pyridin-2-yl)octahydro-1H-pyrrolo[3,2-b]pyridine (300 mg, 54%) and the second eluting isomer (3S,3aS,7aS)-4-(3-fluorophenyl)-3-(2-(2-(2-methoxyethoxy)ethoxy)ethyl)-1-(4-(piperidin-1-yl)pyridin-2-yl)octahydro-1H-pyrrolo[3,2-b]pyridine (290 mg, 52%) as a light yellow oils. LC/MS (ESI, m/z): [(M+H)]⁺=527.4. The absolute stereochemistry of the diastereomers were assigned arbitrarily.

### 2-[(3R,3aR,7aR)-4-(3-fluorophenyl)-3-{2-[2-(2-iodoethoxy)ethoxy]ethyl}-hexahydro-2H-pyrrolo[3,2-b]pyridin-1-yl]-4-(piperidin-1-yl)pyridine (Intermediate OP)

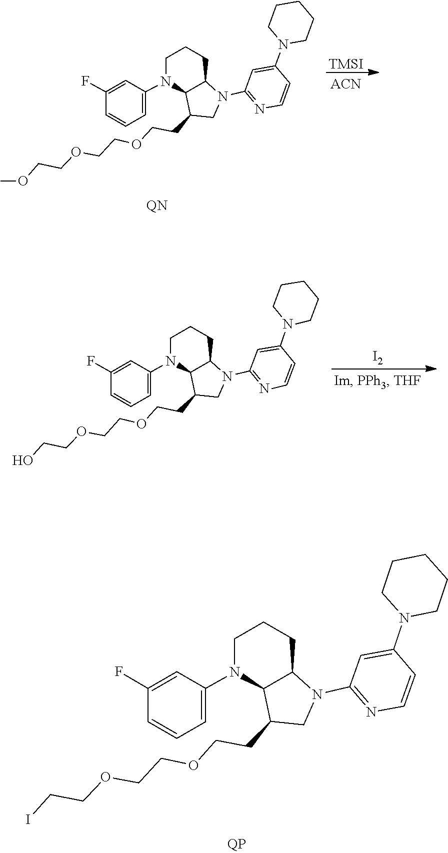

### Step 1—2-(2-{2-[(3aR,7aR)-4-(3-fluorophenyl)-1-[4-(piperidin-1-yl)pyridin-2-yl]-hexahydro-2H-pyrrolo[3,2-b]pyridin-3-yl]ethoxy}ethoxy)ethanol

To a stirred solution of 2-[(3aR,7aR)-4-(3-fluorophenyl)-3-{2-[2-(2-methoxyethoxy)ethoxy]ethyl}-hexahydro-2H-pyrrolo[3,2-b]pyridin-1-yl]-4-(piperidin-1-yl)pyridine (300 mg, 0.57 mmol, Intermediate QN) in CAN (5 mL) was added TMSI (569.86 mg, 2.850 mmol) dropwise at rt under air atmosphere. The resulting mixture was stirred for 24 h at rt under air atmosphere. On completion, the reaction was quenched with water at rt. The aqueous layer was extracted with EtOAc (3×5 mL). The resulting mixture was concentrated under reduced pressure. The residue was purified by Column: XBridge Prep Phenyl OBD Column 19*250 mm, 5 m; Mobile Phase A: Water (10 mmol/L NH4HCO3), Mobile Phase B: ACN; Flow rate: 20 mL/min; Gradient: 58% B to 68% B in 10 min; Wave Length: 254/220 nm: RT1 (min): 8.55 and concentrated under reduced pressure to afford the title compound (150 mg, 51% yield) as a brown oil. LC/MS (ESI, m/z): [(M+1)]=512.7.

### Step 2—2-[(3R,3aR7aR)-4-(3-fluorophenyl)-3-{2-[2-(2-iodoethoxy)ethoxy]ethyl}-hexahydro-2H-pyrrolo[3,2-b]pyridin-1-yl]-4-(piperidin-1-yl)pyridine

To a stirred mixture of 2-(2-{2-[(3R,3aR,7aR)-4-(3-fluorophenyl)-1-[4-(piperidin-1-yl)pyridin-2-yl]-hexahydro-2H-pyrrolo[3,2-b]pyridin-3-yl]ethoxy}ethoxy)ethanol (200 mg, 0.39 mmol) and Imidazole (39.84 mg, 0.585 mmol) in THF (5 mL) were added PPh₃ (153.49 mg, 0.585 mmol) and 12 (148.52 mg, 0.585 mmol) in portions at rt under air atmosphere. The resulting mixture was stirred for 1 h at rt under air atmosphere. On completion, the reaction was quenched with sat. Na₂SO₅ (aq.) at rt. The aqueous layer was extracted with EtOAc (3×5 mL). The resulting mixture was concentrated under reduced pressure. The residue was purified by reverse-phase flash chromatography (column, C18 silica gel; mobile phase, MeOH in Water (10 mmol/L NH₄HCO₃), 50% to 90% gradient in 40 min; detector, UV 254 nm) to give the title compound (120 mg, 49% yield) as a yellow solid. LC/MS (ESI, m/z): [(M+1)]⁺=622.2.

### (3S,3aS,7aS)-4-(3-fluorophenyl)-3-(2-(2-(2-iodoethoxy)ethoxy)ethyl)-1-(4-(piperidin-1-yl)pyridin-2-yl)octahydro-1H-pyrrolo[3,2-b]pyridine (Intermediate QQ)

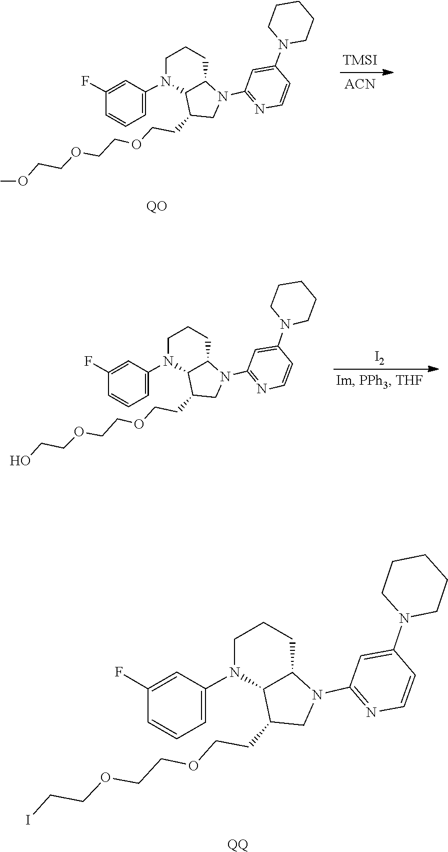

### Step 1—2-(2-(2-((3S,3aS,7aS)-4-(3-fluorophenyl)-1-(4-(piperidin-1-yl)pyridin-2-yl)octahydro-1H-pyrrolo[3,2-b]pyridin-3-yl)ethoxy)ethoxy)ethan-1-ol

To a stirred solution of (3S,3aS,7aS)-4-(3-fluorophenyl)-3-(2-(2-(2-methoxyethoxy)ethoxy)ethyl)-1-(4-(piperidin-1-yl)pyridin-2-yl)octahydro-1H-pyrrolo[3,2-b]pyridine (300 mg, 0.57 mmol, Intermediate QO) in CAN (5 mL) was added TMSI (569.86 mg, 2.850 mmol) dropwise at rt under air atmosphere. The resulting mixture was stirred for 24 h at rt under air atmosphere. On completion, the reaction was quenched with water at rt. The aqueous layer was extracted with EtOAc (3×5 mL). The resulting mixture was concentrated under reduced pressure. The residue was purified by Column: XBridge Prep Phenyl OBD Column 19*250 mm, 5 m; Mobile Phase A: Water (10 mmol/L NH4HCO3), Mobile Phase B: ACN; Flow rate: 20 mL/min: Gradient: 58% B to 68% B in 10 min; Wave Length: 254/220 nm; RT1 (min): 8.55 and concentrated under reduced pressure to afford the title compound (150 mg, 51% yield) as a brown oil. LC/MS (ESI, m/z): [(M+1)]⁺=512.7.

### Step 2—(3S,3aS,7aS)-4-(3-fluorophenyl)-3-(2-(2-(2-iodoethoxy)ethoxy)ethyl)-1-(4-(piperidin-1-yl)pyridin-2-yl)octahydro-1H-pyrrolo[3,2-b]pyridine

To a stirred mixture of 2-(2-(2-((3S,3aS,7aS)-4-(3-fluorophenyl)-1-(4-(piperidin-1-yl)pyridin-2-yl)octahydro-1H-pyrrolo[3,2-b]pyridin-3-yl)ethoxy)ethoxy)ethan-1-ol (200 mg, 0.39 mmol) and Imidazole (39.84 mg, 0.585 mmol) in THF (5 mL) were added PPh₃ (153.49 mg, 0.585 mmol) and I₂ (148.52 mg, 0.585 mmol) in portions at rt under air atmosphere. The resulting mixture was stirred for 1 h at rt under air atmosphere. On completion, the reaction was quenched with sat. Na₂SO₅ (aq.) at rt. The aqueous layer was extracted with EtOAc (3×5 mL). The resulting mixture was concentrated under reduced pressure. The residue was purified by reverse-phase flash chromatography (column, C18 silica gel; mobile phase, MeOH in Water (10 mmol/L NH₄HCO₃), 50% to 90% gradient in 40 min: detector, UV 254 nm) to give the title compound (120 mg, 49% yield) as a yellow solid. LC/MS (ESI, m/z): [(M+1)]⁺=622.2.

### 4-(3,6-Difluoro-2-methylphenyl)-5-(4-(8-hydroxyoctyl)benzoyl)-1-methyl-1H-pyrrole-3-carboxylic acid (Intermediate QR)

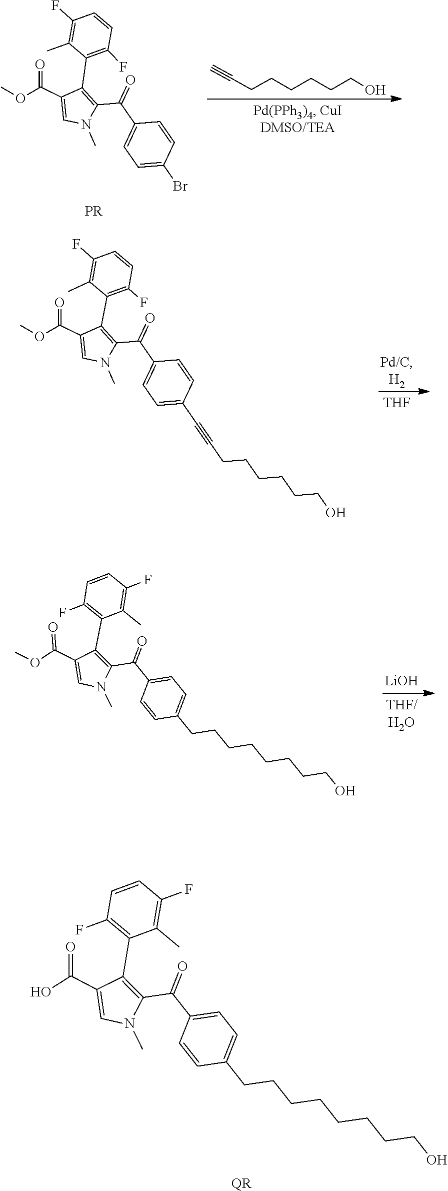

### Step 1—Methyl 4-(3,6-difluoro-2-methylphenyl)-5-(4-(8-hydroxyoct-1-yn-1-yl)benzoyl)-1-methyl-1H-pyrrole-3-carboxylate

To a stirred solution of methyl 5-(4-bromobenzoyl)-4-(3,6-difluoro-2-methylphenyl)-1-methylpyrrole-3-carboxylate (450 mg, 1.00 mmol, Intermediate PR) and oct-7-yn-1-ol (253.38 mg, 2.008 mmol) in DMSO (10 mL) then added TEA (2.5 mL) in portions at rt. To the above mixture was added CuI (38.24 mg, 0.201 mmol) in portions at rt. The resulting mixture was stirred for 2 h at 80° C. under nitrogen atmosphere. On completion, the mixture was cooled to rt and diluted with water (100 mL), then extracted with EtOAc (3×100 mL). The combined organic layers were washed with brine (1×100 mL), and dried over anhydrous Na₂SO₄. After filtration, the filtrate was concentrated under reduced pressure. The residue was purified by silica gel column chromatography, eluted with PE/EA (1:1) to afford the title compound (450 mg, 91% yield) as a brown oil. ¹H NMR (400 MHz, DMSO-d₆) δ 8.03 (s, 1H), 7.39-7.32 (m, 2H), 7.20-7.13 (m, 2H), 6.94-6.83 (m, 1H), 6.78-6.67 (m, 1H), 4.38-4.34 (m, 1H), 3.92 (s, 3H), 3.59 (s, 3H), 3.44-3.36 (m, 2H), 2.42 (t, J=7.0 Hz, 2H), 1.87 (d, J=2.5 Hz, 3H), 1.59-1.39 (m, 4H), 1.43-1.36 (m, 2H), 1.40-1.25 (m, 2H). LC/MS (ESI, m/z): [(M−H)]⁻=492.2.

### Step 2—Methyl 4-(3,6-difluoro-2-methylphenyl)-5-(4-(8-hydroxyoctyl)benzoyl)-1-methyl-1H-pyrrole-3-carboxylate

To a stirred solution of methyl 4-(3,6-difluoro-2-methylphenyl)-5-[4-(8-hydroxyoct-1-yn-1-yl)benzoyl]-1-methylpyrrole-3-carboxylate (450 mg, 0.912 mmol) in THF (20 mL) was added Pd/C (9.70 mg, 0.091 mmol) at rt under nitrogen atmosphere. The resulting mixture was stirred for 1 h at rt under hydrogen atmosphere. On completion, the mixture was filtered, the filter cake was washed with MeOH (2×10 mL). The filtrate was concentrated under reduced pressure to give the title compound (400 mg) as a white solid. ¹H NMR (400 MHz, DMSO-d₆) δ 8.00 (s, 1H), 7.36-7.29 (m, 2H), 6.98 (d, J=8.1 Hz, 2H), 6.82-6-65 (m, 1H), 4.37-4.28 (m, 1H), 3.91 (s, 3H), 3.59 (s, 3H), 3.39 (td, J=6.4, 5.2 Hz, 2H), 2.47 (t, J=7.4 Hz, 2H), 1.85 (d, J=2.6 Hz, 3H), 1.82-1.72 (m, 4H), 1.32-1.25 (m, 6H), 1.20-1.12 m, 2H). LC/MS (ESI, m/z): [(M+H)]⁺=498.4.

### Step 3—4-(3,6-Difluoro-2-methylphenyl)-5-(4-(8-hydroxyoctyl)benzoyl)-1-methyl-1H-pyrrole-3-carboxylic acid

To a stirred solution of 4-(3,6-difluoro-2-methylphenyl)-5-(4-(8-hydroxyoctyl)benzoyl)-1-methyl-1H-pyrrole-3-carboxylate (400 mg, 0.804 mmol) in THF and H₂O (10 mL) was added LiOH aq. (10 mL, 2N) in portions at rt. The resulting mixture was stirred for 2 h at 80° C. under. On completion, the mixture was cooled to rt and concentrated under reduced pressure. The mixture was acidified to pH 3 with cone. HCl and diluted with water (100 mL), then extracted with EtOAc (3×50 mL). The combined organic layers were washed with brine (1×100 mL), and dried over anhydrous Na₂SO₄. After filtration, the filtrate was concentrated under reduced pressure to give the title compound (320 mg) as a yellow solid. ¹H NMR (300 MHz, DMSO-d₆) δ 11.96 (s, 1H), 7.91 (s, 1H), 7.36-7.27 (m, 2H), 6.98 (d, J=8.1 Hz, 2H), 6.81-6.60 (m, 2H), 4.30 (s, 1H), 3.90 (s, 3H), 3.45-3.35 (m, 2H), 2.50-2.41 (m, 2H), 1.87 (d, J=2.6 Hz, 3H), 1.50-1.37 (m, 4H), 1.29-1.25 (m, 6H), 1.20-1.13 (m, 2H). LC/MS (ESI, m/z): [(M−H)]⁻=482.2.

### 5-(4-(8-Bromooctyl)benzoyl)-N-(1-(3-chlorophenyl)-2-(methylsulfonyl)ethyl)-4-(3,6-difluoro-2-methylphenyl)-1-methyl-1H-pyrrole-3-carboxamide (Intermediate OS)

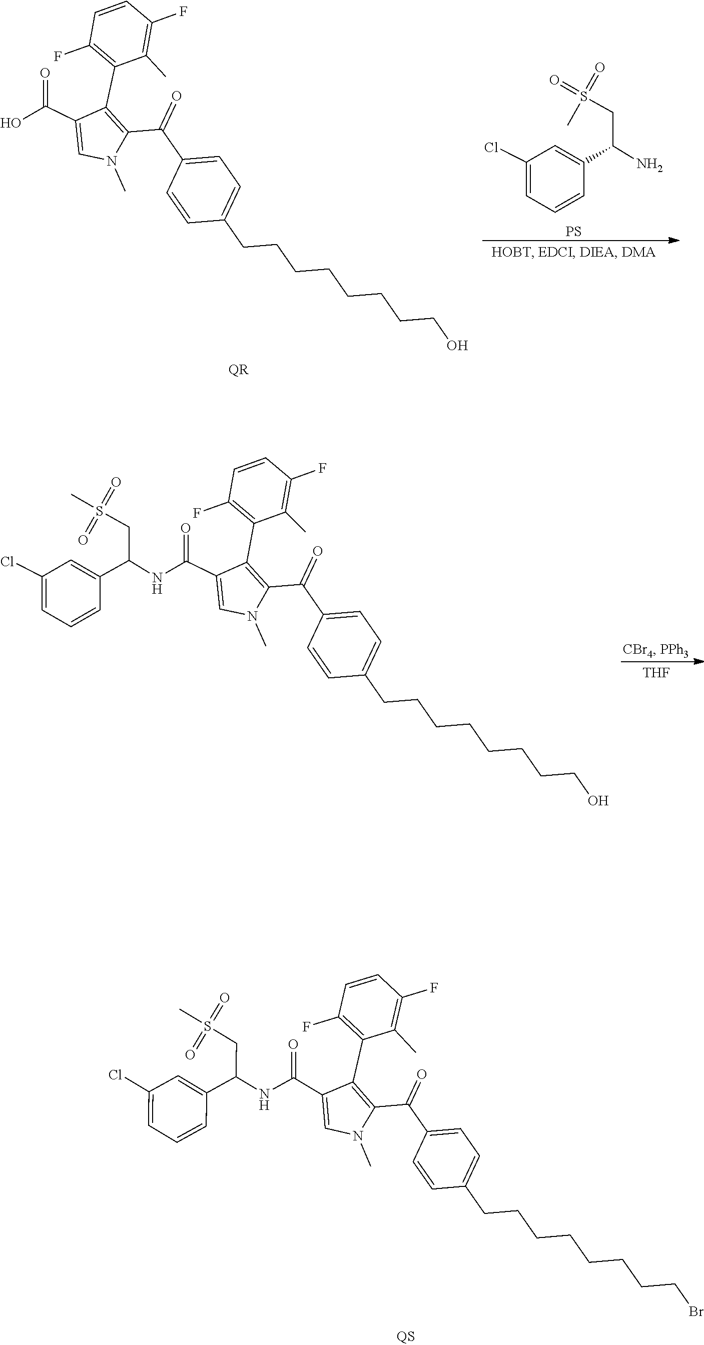

### Step 1—N-(1-(3-chlorophenyl)-2-(methylsulfonyl)ethyl)-4-(3,6-difluoro-2-methylphenyl)-5-(4-(8-hydroxyoctyl)benzoyl)-1-methyl-1H-pyrrole-3-carboxamide

To a stirred solution of 4-(3,6-difluoro-2-methylphenyl)-5-(4-(8-hydroxyoctyl)benzoyl)-1-methyl-1H-pyrrole-3-carboxylic acid (320 mg, 0.662 mmol, Intermediate QR) in DMA (5 mL) were added HOBT (134.13 mg, 0.993 mmol) and EDCI (190.29 mg, 0.993 mmol) in portions at rt. To the above mixture was added (1R)-1-(3-chlorophenyl)-2-methanesulfonylethanamine (231.99 mg, 0.993 mmol, Intermediate PS) in portions at rt. The resulting mixture was stirred for 16 h at rt. On completion, the mixture was concentrated under reduced pressure. The residue was purified by reverse phase flash chromatography (Column: WelFlash™ C18-I, 20-40 um, 120 g; Eluent A: Water (plus 10 mmol/L NH₄HCO₃); Eluent B: ACN; Gradient: 25%-75% B in 35 min; Flow rate: 80 mL/min; Detector: 220/254 nm; desired fractions were collected at 69% B and concentrated under reduced pressure) to afford the title compound (300 mg, 65% yield) as a brown oil. ¹H NMR (400 MHz, DMSO-d₆) δ 8.56-8.46 (m, 1H), 7.85 (d, J=7.5 Hz, 1H), 7.49-7.41 (m, 1H), 7.40-7.27 (m, 3H), 7.30-7.24 (m, 2H), 6.96 (d, J=7.8 Hz, 2H), 6.74-6.51 (m, 2H), 5.50-5.34 (m, 1H), 4.32 (t, J=5.1 Hz, 1H), 3.91 (s, 3H), 3.73-3.55 (m, 2H), 3.43-3.34 (m, 2H), 2.97 (d, J=9.5 Hz, 3H), 2.46 (t, J=7.5 Hz, 2H), 1.87 (d, J=44.0 Hz, 3H), 1.45-1.38 (m, 4H), 1.30-1.26 (m, 6H), 1.19-1.13 (m, 2H); LC/MS (ESI, m/z): [(M−H)]⁻=697.1.

### Step 2—5-(4-(8-Bromooctyl)benzoyl)-N-(1-(3-chlorophenyl)-2-(methylsulfonyl)ethyl)-4-(3,6-difluoro-2-methylphenyl)-1-methyl-1H-pyrrole-3-carboxamide

To a stirred solution of N-(1-(3-chlorophenyl)-2-(methylsulfonyl)ethyl)-4-(3,6-difluoro-2-methylphenyl)-5-(4-(8-hydroxyoctyl)benzoyl)-1-methyl-1H-pyrrole-3-carboxamide (200 mg, 0.286 mmol) in THF (10 mL) were added PPh₃ (150.04 mg, 0.572 mmol) and DEAD (85.37 mg, 0.572 mmol) in portions at rt. To the above mixture was added CBr₄ (474.26 mg, 1.430 mmol) in portions at rt. The resulting mixture was stirred for 2 h at rt under nitrogen atmosphere. On completion, the mixture was concentrated under reduced pressure. The residue was purified by silica gel column chromatography, eluted with PE/EA (1:1), to afford the title compound (200 mg, 92% yield) as a yellow oil. ¹H NMR (400 MHz, DMSO-d₆) δ 8.54-8.45 (m, 1H), 7.85 (d, J=7.6 Hz, 1H), 7.47-7.40 (m, 1H), 7.38-7.30 (m, 3H), 7.28 (d, J=7.8 Hz, 2H), 6.96 (d, J=7.8 Hz, 2H), 6.75-6.54 (m, 2H), 5.41-5.35 (m, 1H), 3.91 (s, 3H), 3.72-3.57 (in, 2H), 3.53 (t, J=6.7 Hz, 2H), 2.97 (d, J=9.5 Hz, 3H), 2.46 (t, J=7.3 Hz, 2H), 1.81 (d, J=56.0 Hz, 3H), 1.85-1.76 (m, 2H), 1.47-1.36 (m, 4H), 1.30-1.26 (m, 4H), 1.17-1.13 (m, 2H); LC/MS (ESI, m/z): [(M+H)]⁺=761.2.

### 4-[8-(Oxan-2-yloxy)octyl]piperidine (Intermediate OT)

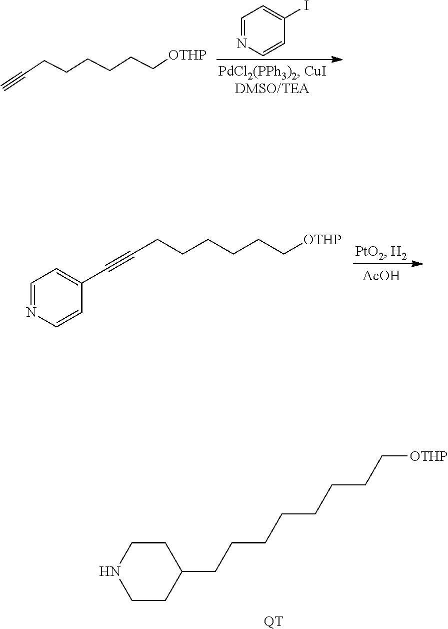

### Step 1—4-[8-(oxan-2-yloxy)oct-1-yn-1-yl]pyridine

To a stirred solution of 4-iodopyridine (10 g, 50 mmol, CAS #15854-87-2) and 2-(oct-7-yn-1-yloxy)oxane (12.31 g, 58.54 mmol, CAS: 16695-31-1) in DMSO (100 mL) and TEA (20 mL) was added Pd(PPh₃)₂Cl₂ (3.42 g, 4.88 mmol) at rt. The resulting mixture was stirred for 1 h at 80° C. under nitrogen atmosphere. On completion, the mixture was cooled to rt and diluted with water (200 mL). The resulting mixture was extracted with EtOAc (3×200 mL). The combined organic layers were washed with brine (3×200 mL), and dried over anhydrous Na₂SO₄. After filtration, the filtrate was concentrated under reduced pressure. The residue was purified by silica gel column chromatography, eluted with PE/EA (5:1), to afford the title compound (12.6 g, 90% yield) as a brown oil. LC/MS (ESI, m/z): [(M+H)]⁺=288.3.

### Step 2—4-[8-(Oxan-2-yloxy)octyl]piperidine

To a stirred solution of 4-[8-(oxan-2-yloxy)oct-1-yn-1-yl]pyridine (12.6 g, 43.8 mmol) in AcOH (150 mL) was added PtO₂ (4.98 g, 21.9 mmol) at rt. The reaction system was degassed under vacuum and purged with H₂ several times, then the mixture was hydrogenated under H₂ balloon (1 atm) at 25° C. for 16 h. After completion of the reaction, PtO₂ was filtered off through celite and the corresponding filtrate was concentrated under reduced pressure to afford the title compound (11 g, 84% yield) as a black oil. LC/MS (ESI, m/z): [(M+H)]⁺=298.3.

### 2-Bromo-4-{4-[8-(oxan-2-yloxy)octyl]piperidin-1-yl}pyridine (Intermediate QU)

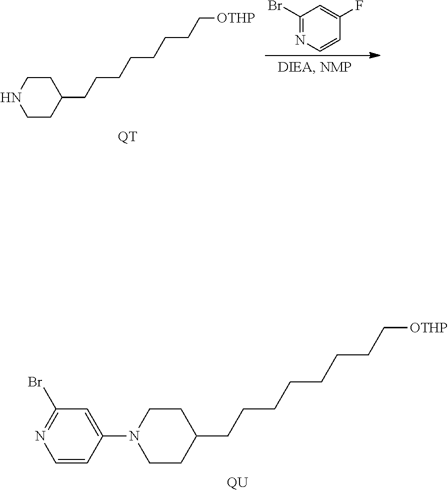

To a stirred solution of 4-[8-(oxan-2-yloxy)octyl]piperidine (3 g, 10 mmol, Intermediate QT) and 2-bromo-4-fluoropyridine (2.13 g, 12.1 mmol, CAS #357927-50-5) in NMP (30 mL) was added DIEA (3.53 ml, 20.2 mmol) at rt. The resulting mixture was stirred for 1 h at 130° C. On completion, the mixture was cooled to rt and purified by reverse phase flash chromatography (Column: WelFlash™ C18-I, 20-40 um, 330 g; Eluent A: Water (plus 10 mmol/L NH₄CO₃): Eluent B: MeOH; Gradient: 50%-95% B in 45 min: Flow rate: 80 mL/min; Detector: 220/254 nm; desired fractions were collected at 92% B and concentrated under reduced pressure) to afford the title compound (2.4 g, 53% yield) as a brown oil. LC/MS (ESI, m/z): [(M+H)]⁺=453.20, LC/MS (ESI, m/z): [(M+H+2)]⁺=455.2.

### 2-[(3aR,7aR)-4-(3-fluorophenyl)-hexahydro-2H-pyrrolo[3,2-b]pyridin-1-yl]-4-[4-(8-iodooctyl)piperidin-1-yl]pyridine (Intermediate QV)

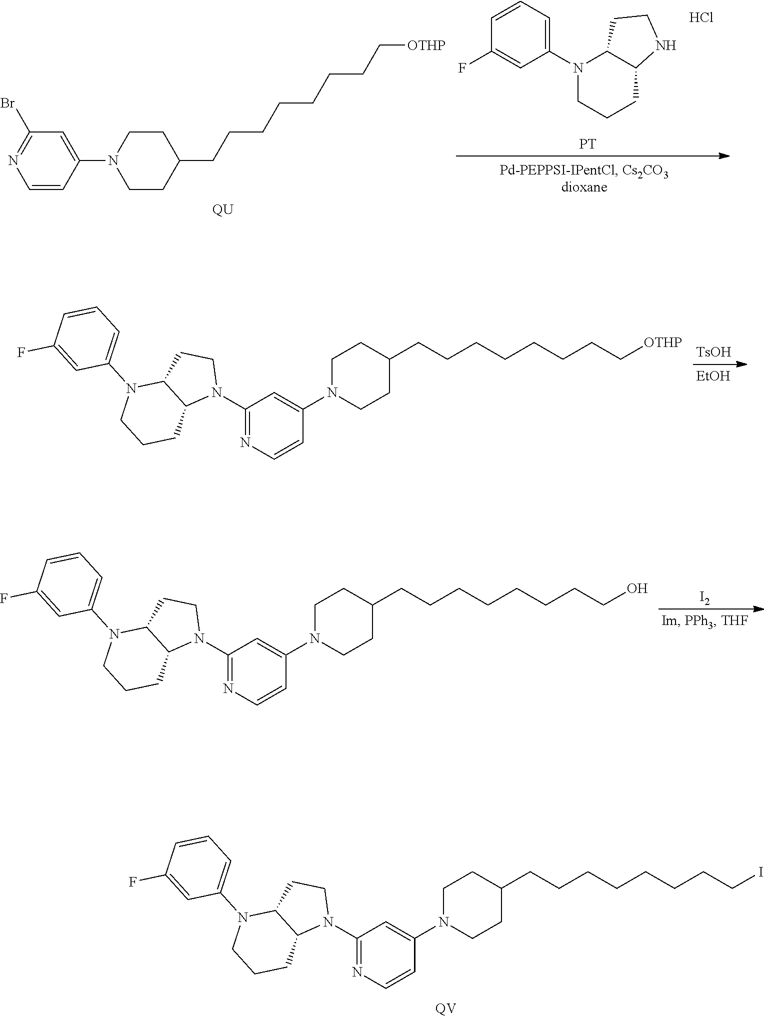

### Step 1—2-[(3aR,7aR)-4-(3-fluorophenyl)-hexahydro-2H-pyrrolo[3,2-b]pyridin-1-yl]-4-{4-[8-(oxan-2-yloxy)octyl]piperidin-1-yl}pyridine

To a stirred solution of 2-bromo-4-{4-[8-(oxan-2-yloxy)octyl]piperidin-1-yl}pyridine (1.03 g, 2.27 mmol, Intermediate QU) and (3aR,7aR)-4-(3-fluorophenyl)-octahydropyrrolo[3,2-b]pyridine hydrochloride (0.50 g, 2.27 mmol, Intermediate PT) in 1,4-dioxane (10 mL) were added Pd-PEPPSI-IPentCl (0.19 g, 0.23 mmol) and Cs₂CO₃ (1.48 g, 4.54 mmol) at rt. The resulting mixture was stirred for 16 h at 100° C. under nitrogen atmosphere. On completion, the mixture was cooled to rt and concentrated under reduced pressure. The residue was purified by reverse phase Flash chromatography (Column: WelFlash™ C18-I, 20-40 um, 330 g; Eluent A: Water (plus 10 mmol/L TFA): Eluent B: ACN; Gradient: 30%-70% B in 40 min; Flow rate: 80 mL/min; Detector: 220/254 nm: desired fractions were collected at 64% B and concentrated under reduced pressure) to afford the title compound (580 mg, 43% yield) as a brown oil. LC/MS (ESI, m/z): [(M+H)]⁺=593.4.

### Step 2—8-(1-{2-[(3aR,7aR)-4-(3-fluorophenyl)-hexahydro-2H-pyrrolo[3,2-b]pyridin-1-yl]pyridin-4-yl}piperidin-4-yl)octan-1-ol

To a stirred solution of 2-[(3aR,7aR)-4-(3-fluorophenyl)-hexahydro-2H-pyrrolo[3,2-b]pyridin-1-yl]-4-{4-[8-(oxan-2-yloxy)octyl]piperidin-1-yl}pyridine (580 mg, 0.978 mmol) in EtOH (10 mL) was added TsOH·H₂O (186.09 mg, 0.978 mmol) at rt. The resulting mixture was stirred for 1 h at 80° C. On completion, the mixture was cooled to rt and concentrated under reduced pressure. The residue was purified by reverse phase Flash chromatography (Column: WelFlash™ C18-I, 20-40 um, 330 g; Eluent A: Water (plus 10 mmol/L NH₄CO₃); Eluent B: MeOH: Gradient: 50%-95% B in 45 min; Flow rate: 80 mL/min; Detector: 220/254 nm; desired fractions were collected at 95% B and concentrated under reduced pressure) to afford the title compound (480 mg, 96% yield) as a brown oil. LC/MS (ESI, m/z): [(M+H)]⁺=509.4.

### Step 3—2-[(3aR,7aR)-4-(3-fluorophenyl)-hexahydro-2H-pyrrolo[3,2-b]pyridin-1-yl]-4-[4-(8-iodooctyl)piperidin-1-yl]pyridine

To a stirred solution of 8-(1-{2-[(3aR,7aR)-4-(3-fluorophenyl)-hexahydro-2H-pyrrolo[3,2-b]pyridin-1-yl]pyridin-4-yl}piperidin-4-yl)octan-1-ol (480 mg, 0.944 mmol) and imidazole (128.47 mg, 1.888 mmol) in THF (10 mL) were added PPh₃ (494.96 mg, 1.888 mmol) and 12 (478.95 mg, 1.888 mmol) at 0° C. The resulting mixture was then stirred for 1 h at rt under nitrogen atmosphere. On completion, the mixture was concentrated under reduced pressure. The residue was purified by reverse phase flash chromatography (Column: WelFlash™ C18-I, 20-40 urn, 330 g; Eluent A: Water (plus 10 mmol/L NH₄CO₃); Eluent B: MeOH; Gradient: 50%-100% B in 60 min; Flow rate: 80 mL/min; Detector: 220/254 nm; desired fractions were collected at 100% B and concentrated under reduced pressure) to afford the title compound (290 mg, 50% yield) as a brown oil. LC/MS (ESI, m/z): [(M+H)]=619.2.

### 2-Chloro-3-(1-phenyl-2,5,8,11,14-pentaoxahexadecan-16-yl)-4-(piperidin-1-yl)pyridine (Intermediate OW)

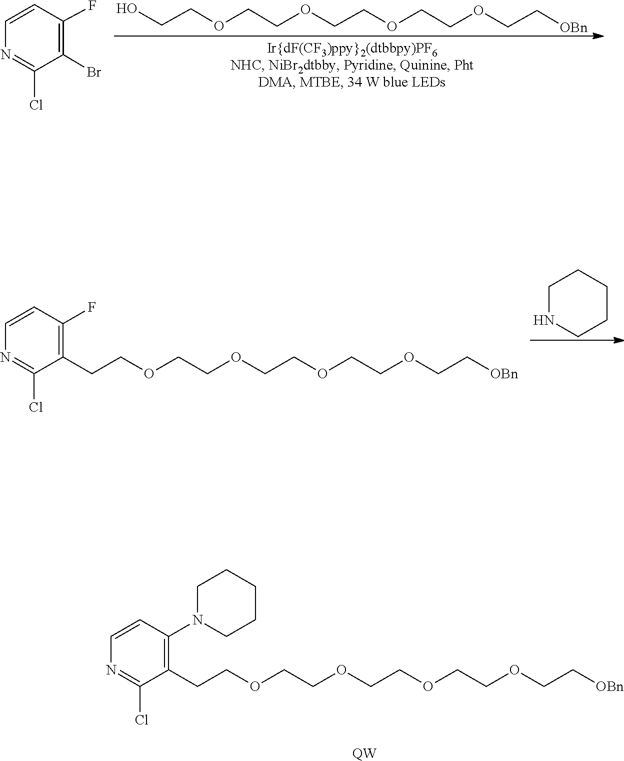

### Step 1—2-Chloro-4-fluoro-3-(1-phenyl-2,5,8,11,14-pentaoxahexadecan-16-yl)pyridine

To a stirred solution of 1-phenyl-2,5,8,11,14-pentaoxahexadecan-16-ol (7.02 g, 21.4 mmol, CAS #57671-28-0) in MTBE (15 mL) was added 5,7-di-tert-butyl-3-phenyl-1,3lambda5-benzoxazol-3-ylium tetrafluoroborate (8.45 g, 21.4 mmol) at rt. The resulting mixture was stirred for 20 min at rt under nitrogen atmosphere. To the above mixture was added pyridine (1127.69 mg, 14.257 mmol) at rt. The resulting mixture was stirred for additional 30 min at rt. The resulting mixture was filtered, the filter cake was washed with TBME. The filtrate was collected. To the above solution were added 3-bromo-2-chloro-4-fluoropyridine (3 g, 14 mmol, CAS #1349717-04-9), NiBr₂dtbby (694.15 mg, 1.426 mmol), Ir{dF(CF3)ppy}₂(dtbbpy)PF₆ (1602.33 mg, 1.426 mmol), 1-azabicyclo[2.2.2]octane (2773.94 mg, 24.950 mmol) and 2,3-dihydro-1H-isoindole-1,3-dione (419.51 mg, 2.851 mmol) in DMA (6 mL) at rt. The vial was stirred at 1500 rpm stir rate and irradiated under 450 nm LED modules at 100% light intensity with maxed fan speed of 1500 rpm stirring rate in a PennOC Integrated Photoreactor for 2 hrs. On completion, the mixture was concentrated under reduced pressure. The residue was purified by reverse phase flash chromatography (Column: WelFlash™ C18-I, 20-40 un, 330 g; Eluent A: Water (plus 10 mmol/L NH₄HCO₃); Eluent B: ACN; Gradient: 30%-80% B in 30 min: Flow rate: 85 mL/min; Detector: 220 nm; desired fractions were collected at 66% B and concentrated under reduced pressure) to afford the title compound (400 mg, 6% yield) as a light yellow oil. LC/MS (ESI, m/z): [(M+H)]⁺=442.2.

### Step 2—2-Chloro-3-(1-phenyl-2,5,8,11,14-pentaoxahexadecan-16-yl)-4-(piperidin-1-yl)pyridine

A stirred solution of 2-chloro-4-fluoro-3-(1-phenyl-2,5,8,11,14-pentaoxahexadecan-16-yl)pyridine (400 mg, 0.905 mmol) in piperidine (4 mL) was stirred for 16 h at 100° C. On completion, the mixture was concentrated under reduced pressure. The residue was purified by reverse phase flash chromatography (Column: WelFlash™ C18-I. 20-40 um, 120 g: Eluent A: Water (plus 10 mmol/L NH₄HCO₃); Eluent B: ACN: Gradient: 25%-60% B in 30 min: Flow rate: 65 mL/min; Detector: 220 nm: desired fractions were collected at 56% B and concentrated under reduced pressure) to afford the title compound (350 mg, 76% yield) as a light yellow oil. LC/MS (ESI, m/z): [(M+H)]⁺=507.3.

### 2-[(3aR,7aR)-4-(3-fluorophenyl)-hexahydro-2H-pyrrolo[3,2-b]pyridin-1-yl]-3-(14-iodo-3,6,9,12-tetraoxatetradecan-1-yl)-4-(piperidin-1-yl)pyridine (Intermediate QX)

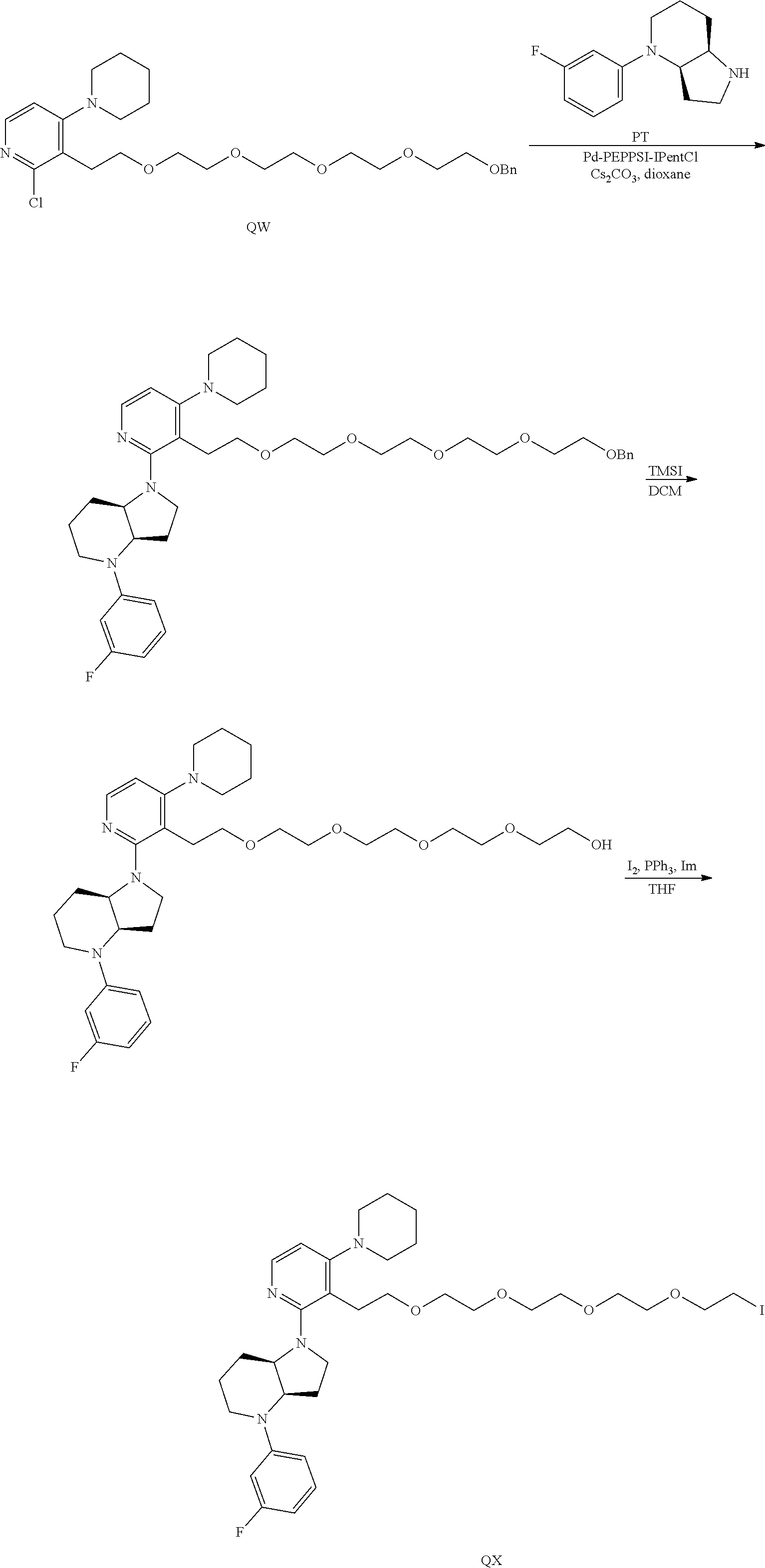

### Step 1—2-[(3aR,7aR)-4-(3-fluorophenyl)-hexahydro-2H-pyrrolo[3,2-b]pyridin-1-yl]-3-(1-phenyl-2,5,8,11,14-pentaoxahexadecan-16-yl)-4-(piperidin-1-yl)pyridine

To a stirred mixture of 2-chloro-3-(1-phenyl-2,5,8,11,14-pentaoxahexadecan-16-yl)-4-(piperidin-1-yl)pyridine (350 mg, 0.690 mmol, Intermediate QW) and (3aR,7aR)-4-(3-fluorophenyl)-octahydropyrrolo[3,2-b]pyridine hydrochloride (212.66 mg, 0.828 mmol, Intermediate PT) in dioxane (6 mL) were added Pd-PEPPSI-IPentCl (58.06 mg, 0.069 mmol) and Cs₂CO₃ (674.68 mg, 2.070 mmol) at rt. The resulting mixture was stirred for 16 h at 100° C. under nitrogen atmosphere. On completion, the mixture was cooled to rt and concentrated under reduced pressure. The residue was purified by reverse phase flash chromatography (Column: WelFlash™ C18-I, 20-40 um, 120 g; Eluent A: Water (plus 10 mmol/L NH4HCO₃); Eluent B: ACN: Gradient: 25%-60% B in 30 min: Flow rate: 65 mL/min; Detector: 220 nm: desired fractions were collected at 60% B and concentrated under reduced pressure) to afford the title compound (180 mg, 38% yield) as a brown oil. LC/MS (ESI, m/z): [(M+H)]⁺=691.4.

### Step 2—14-{2-[(3aR,7aR)-4-(3-fluorophenyl)-hexahydro-2H-pyrrolo[3,2-b]pyridin-1-yl]-4-(piperidin-1-yl)pyridin-3-yl}-3,6,912-tetraoxatetradecan-1-ol

To a stirred solution of 2-[(3aR,7aR)-4-(3-fluorophenyl)-hexahydro-2H-pyrrolo[3,2-b]pyridin-1-yl]-3-(1-phenyl-2,5,8,11,14-pentaoxahexadecan-16-yl)-4-(piperidin-1-yl)pyridine (60 mg, 0.087 mmol) in DCM (2 mL) was added TMSI (34.75 mg, 0.174 mmol) at rt. The resulting mixture was stirred for 2 h at rt. On completion, the mixture was concentrated under reduced pressure. The residue was purified by reverse phase flash chromatography (Column: WelFlash™ C18-I, 20-40 um, 120 g; Eluent A: Water (plus 10 mmol/L FA): Fluent B: MeOH; Gradient: 25%-65% B in 30 min; Flow rate: 85 mL/min: Detector: 220 nm; desired fractions were collected at 45% B) and concentrated under reduced pressure. The residue was then purified by Column: (Xcelect CSH F-pheny OBD Column 19*250 mm, 5 m; Mobile Phase A: Water (0.1% FA), Mobile Phase B: ACN; Flow rate: 25 mL/min; Gradient: 15% B to 23% B in 10 min; Wave Length: 254/220 nm; RT1 (min): 19) to afford the title compound (47 mg, 90% yield) as a light yellow solid. ¹H NMR (400 MHz, DMSO-d₆) δ 7.89 (d, J=5.4 Hz, 1H), 7.27-7.16 (m, 1H), 6.86-6.69 (m, 2H), 6.57-6.47 (m, 2H), 4.47-4.37 (m, 1H), 4.29-4.19 (m, 1H), 3.81-3.61 (m, 2H), 3.57-3.44 (m, 15H), 3.31-3.23 (m, 4H), 3.20-2.98 (m, 4H), 2.95-2.87 (m, 2H), 2.83-2.66 (m, 3H), 2.07-1.94 (m, 1H), 1.87-1.46 (m, 9H). LC/MS (ESI, m/z): [(M+H)]⁺=601.2.

### Step 3—2-[(3aR,7aR)-4-(3-fluorophenyl)-hexahydro-2H-pyrrolo[3,2-b]pyridin-1-yl]-3-(14-iodo-3,6,9,12-tetraoxatetradecan-1-yl)-4-(piperidin-1-yl)pyridine

To a stirred mixture of 14-{2-[(3aR,7aR)-4-(3-fluorophenyl)-hexahydro-2H-pyrrolo[3,2-b]pyridin-1-yl]-4-(piperidin-1-yl)pyridin-3-yl}-3,6,9,12-tetraoxatetradecan-1-ol (80 mg, 0.13 mmol) in THF (4 mL) were added PPh₃ (52.39 mg, 0.200 mmol) and imidazole (13.60 mg, 0.200 mmol) at rt. The resulting mixture was stirred for 15 min at rt. To the above mixture was added I₂ (67.59 mg, 0.266 mmol) at rt. The resulting mixture was stirred for additional 1 h at rt. On completion, the mixture was concentrated under reduced pressure. The residue was purified by reverse phase flash chromatography (Column: WelFlash™ C18-I, 20-40 um, 120 g; Eluent A: Water (plus 10 mmol/L NH₄HCO₃); Eluent B: MeOH: Gradient: 30%-95% B in 30 min; Flow rate: 60 mL/min; Detector: 220 nm; desired fractions were collected at 95% B and concentrated under reduced pressure) to afford the title compound (90 mg, 95% yield) as a colorless oil. LC/MS (ESI, m/z): [(M+H)]⁺=711.2.

### 4-(3,6-Difluoro-2-methylphenyl)-5-[4-(14-hydroxy-3,6,9,12-tetraoxatetradecan-1-yl)benzoyl]-1H-pyrrole-3-carboxylic acid (Intermediate QY)

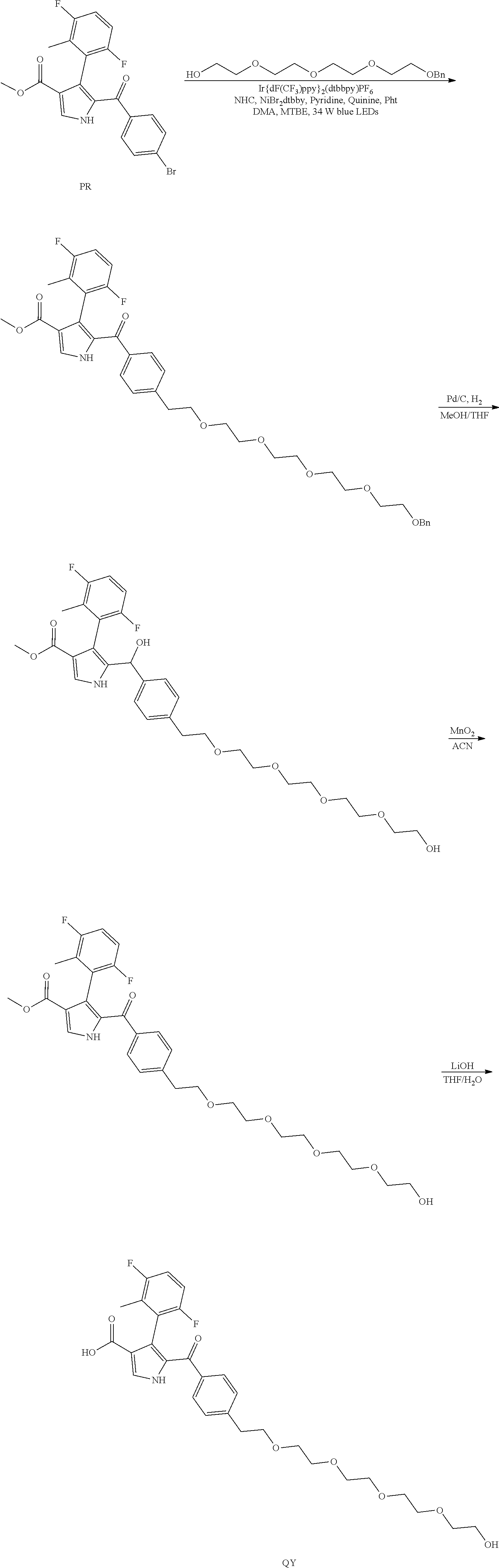

### Step 1—4-(3,6-Difluoro-2-methylphenyl)-5-[4-(1-phenyl-2,5,8,11,14,17-hexaoxanonadecan-19-yl)benzoyl]-1H-pyrrole-3-carboxylate

To a stirred mixture of 1-phenyl-2,5,8,11,14-pentaoxahexadecan-16-ol (2.27 g, 6.91 mmol, CAS #86259-87-2) in 2-methoxy-2-methylpropane (2 mL) were added 5,7-di-tert-butyl-3-phenyl-1,3lambda5-benzoxazol-3-ylium boron trifluoride fluoride (1.46 g, 3.69 mmol) and pyridine (291.45 mg, 3.685 mmol) at rt under nitrogen atmosphere. The mixture was stirred for 20 min at rt under nitrogen atmosphere. The resulting mixture was filtered, the filter cake was washed with 2-methoxy-2-methylpropane (2 mL). The filtrate was then used directly. To a stirred mixture of methyl 5-(4-bromobenzoyl)-4-(3,6-difluoro-2-methylphenyl)-1H-pyrrole-3-carboxylate (1.00 g, 2.30 mmol, Intermediate PR) and photocatalyst (315.71 mg, 0.345 mmol) in DMA (10 mL) were added the nickel catalyst (56.06 mg, 0.115 mmol) and 2,3-dihydro-1H-isoindole-1,3-dione (67.77 mg, 0.461 mmol) at rt under nitrogen atmosphere. To the above mixture was added the filtrate at rt. The vial was stirred at 1500 rpm stir rate and irradiated under 450 nm LED modules at 100% light intensity in a PennOC Integrated Photoreactor for 2 hr. On completion, the mixture was concentrated under reduced pressure. The residue was purified by reverse-phase flash chromatography (column, C18 silica gel; mobile phase, MeCN in Water (0.1% FA), 30% to 70% gradient in 30 min; detector, UV 254 nm) to give the title compound (700 mg, 43% yield) as a light yellow solid. LC/MS (ESI, m/z): [(M+H)]⁺=666.0.

### Step 2—Methyl 4-(3,6-difluoro-2-methylphenyl)-5-{hydroxy[4-(14-hydroxy-3,6,9,12-tetraoxatetradecan-1-yl)phenyl]methyl}-1H-pyrrole-3-carboxylate

A solution of methyl 4-(3,6-difluoro-2-methylphenyl)-5-[4-(1-phenyl-2,5,8,11,14-pentaoxahexadecan-16-yl)benzoyl]-1H-pyrrole-3-carboxylate (700 mg, 1.051 mmol, 1 equiv) and Pd/C (112 mg, 1.05 mmol) in MeOH (10 mL) was stirred for 2 h at rt under hydrogen atmosphere. On completion, the mixture was filtered, and the filter cake was washed with MeOH (3×100 mL). The filtrate was concentrated under reduced pressure to give the title compound (600 mg, 99% yield) as a light yellow oil. LC/MS (ESI, m/z): [(M+H)]⁺=578.0.

### Step 3—Methyl 4-(3,6-difluoro-2-methylphenyl)-5-[4-(14-hydroxy-3,6,9,12-tetraoxatetradecan-1-yl)benzoyl]-1H-pyrrole-3-carboxylate

A solution of methyl 4-(3,6-difluoro-2-methylphenyl)-5-{hydroxy[4-(14-hydroxy-3,6,9,12-tetraoxatetradecan-1-yl)phenyl]methyl}-1H-pyrrole-3-carboxylate (600 mg, 1.04 mmol) and MnO₂ (903 mg, 10.4 mmol) in ACN (15 mL) was stirred for 2 h at 80° C. On completion, the mixture was cooled to rt and filtered, then the filter cake was washed with MeOH (3×100 mL). The filtrate was concentrated under reduced pressure to give the title compound (400 mg, 67% yield) as a light yellow oil. LC/MS (ESI, m/z): [(M+H)]⁺=576.0.

### Step 4—4-(3,6-difluoro-2-methylphenyl)-5-[4-(14-hydroxy-3,6,9,12-tetraoxatetradecan-1-yl)benzoyl]-1H-pyrrole-3-carboxylic acid

A solution of methyl 4-(3,6-difluoro-2-methylphenyl)-5-[4-(14-hydroxy-3,6,9,12-tetraoxatetradecan-1-yl)benzoyl]-1H-pyrrole-3-carboxylate (400 mg, 0.695 mmol) in LiOH (9 mL) and MeOH (3 mL) was stirred for 2 h at rt. On completion, the mixture was cooled to rt and concentrated under reduced pressure. The mixture was acidified to pH 4 with HCl (aq.). The resulting mixture was extracted with EtOAc (3×100 mL). The combined organic layers were washed with brine (100 mL), and dried over anhydrous Na₂SO₄. After filtration, the filtrate was concentrated under reduced pressure to give the title compound (200 mg, 51% yield) as a light yellow oil. LC/MS (ESI, m/z): [(M+H)]⁺=562.0.

### (3S)-3-(3-chlorophenyl)-3-{[4-(3,6-difluoro-2-methylphenyl)-5-[4-(14-oxo-3,6,9,12-tetraoxatetradecan-1-yl)benzoyl]-1H-pyrrol-3-yl]formamido}propenamide (Intermediate QZ)

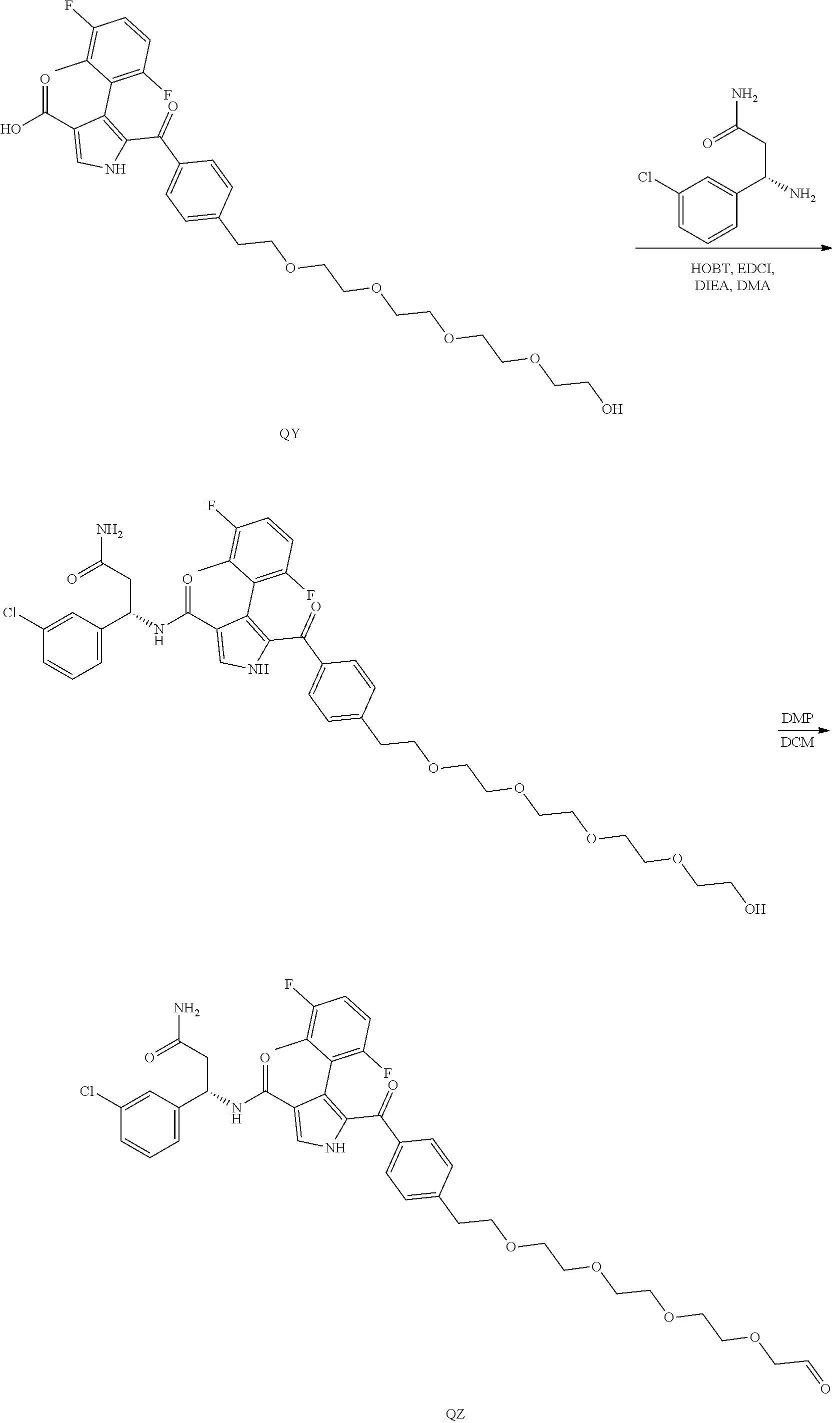

### Step 1—(3S)-3-(3-chlorophenyl)-3-{[4-(3,6-difluoro-2-methylphenyl)-5-[4-(14-hydroxy-3,6,9,12-tetraoxatetradecan-1-yl)benzoyl]-1H-pyrrol-3-yl]formamido}propanamide

To a stirred solution of 4-(3,6-difluoro-2-methylphenyl)-5-[4-(14-hydroxy-3,6,9,12-tetraoxatetradecan-1-yl)benzoyl]-1H-pyrrole-3-carboxylic acid (200 mg, 0.356 mmol, Intermediate QY), DIEA (230 mg, 1.78 mmol) and HATU (203 mg, 0.534 mmol) in DMA (5 mL) was added (3S)-3-amino-3-(3-chlorophenyl)propanamide (106 mg, 0.534 mmol, CAS #1307991-87-2) at rt. The resulting mixture was stirred for 2 h at rt. On completion, the mixture was concentrated under reduced pressure. The residue was purified by reverse phase flash chromatography (Column: WelFlash™ C18-I, 20-40 μm, 120 g; Eluent A: Water (plus 10 mmol/L NH₄HCO₃); Eluent B: ACN; Gradient: 25%-45% B in 25 min; Flow rate: 60 mL/min; Detector: 220/254 nm; desired fractions were collected at 42% B and concentrated under reduced pressure) to give the title compound (190 mg, 72% yield) as a light yellow solid. LC/MS (ESI, m/z): [(M+H)]⁺=742.0.

### Step 2—(3S)-3-(3-chlorophenyl)-3-{[4-(3,6-difluoro-2-methylphenyl)-5-[4-(14-oxo-3,6,9,12-tetraoxatetradecan-1-yl)benzoyl]-1H-pyrrol-3-yl]formamido}propanamide

To a stirred solution of (3S)-3-(3-chlorophenyl)-3-{[4-(3,6-difluoro-2-methylphenyl)-5-[4-(14-hydroxy-3,6,9,12-tetraoxatetradecan-1-yl)benzoyl]-1H-pyrrol-3-yl]formamido}propanamide (60 mg, 0.08 mmol) in DCM (5 mL) was added 1,1-bis(acetyloxy)-3-oxo-3H-1,2-benziodaoxol-1-yl acetate (41 mg, 0.097 mmol) at rt. The resulting mixture was stirred for 2 h at rt. On completion, the mixture was concentrated under reduced pressure. The residue was purified by reverse phase flash chromatography (Column: WelFlash™ C18-I, 20-40 μm, 120 g; Eluent A: Water (plus 10 mmol/L NH₄HCO₃): Eluent B: ACN: Gradient: 25%-45% B in 25 min: Flow rate: 60 m/min; Detector: 220/254 nm; desired fractions were collected at 32% B and concentrated under reduced pressure) to give the title compound (20 mg, 33% yield) as a white solid. LC/MS (ESI, m/z): [(M+H)]=740.0.

### 4-(3,6-Difluoro-2-methylphenyl)-5-[4-(17-hydroxy-3,6,9,12,15-pentaoxaheptadecan-1-yl)benzoyl]-1H-pyrrole-3-carboxylic acid (Intermediate RA)

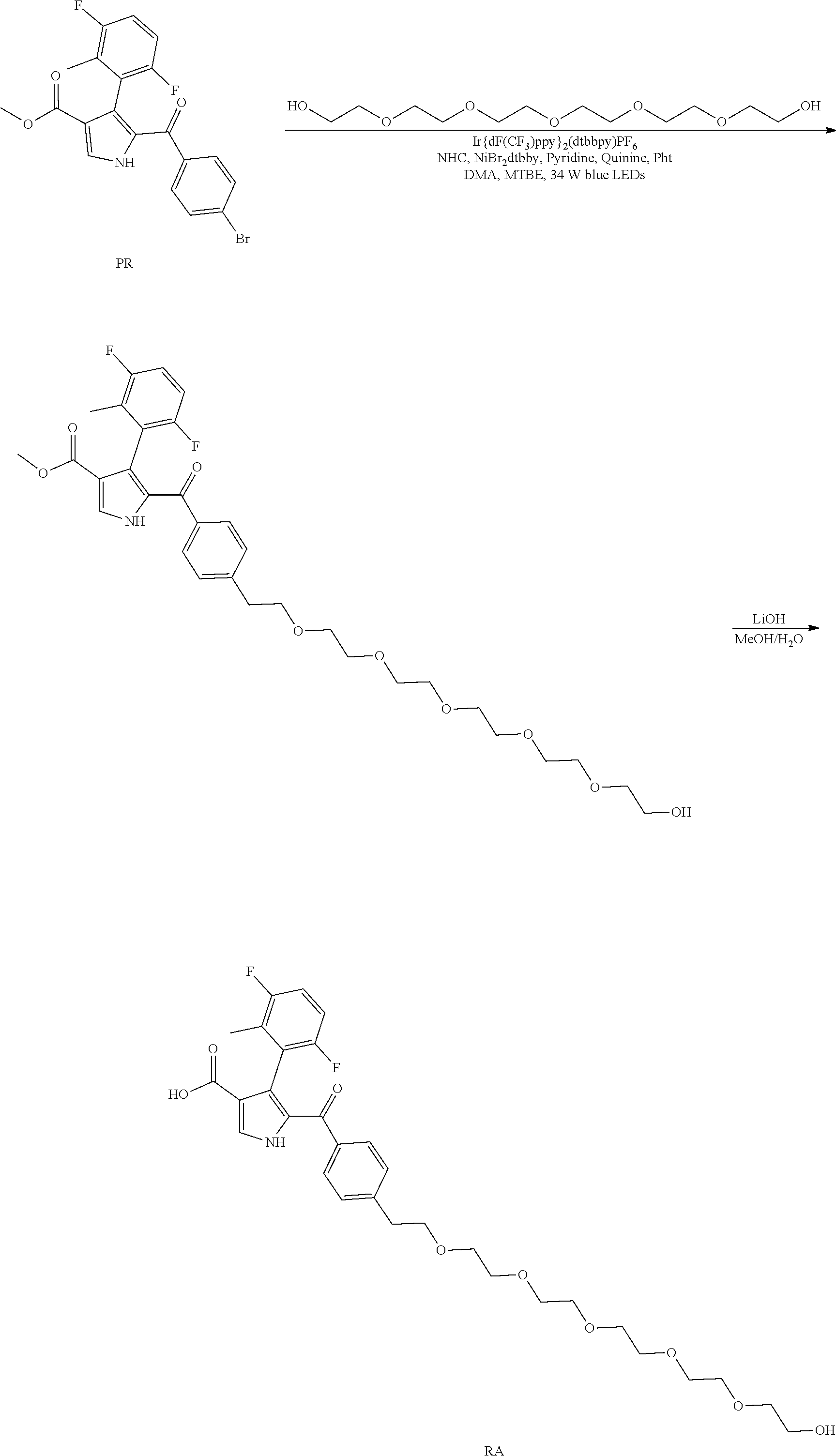 [SMILES: COC(=O)c1c[nH]c(C(=O)c2ccc(Br)cc2)c1-c1c(F)ccc(F)c1C ; OCCOCCOCCOCCOCCOCCO ; COC(=O)c1c[nH]c(C(=O)c2ccc(CCOCCOCCOCCOCCOCCO)cc2)c1-c1c(F)ccc(F)c1C ; "LiOH" ; Cc1c(F)ccc(F)c1-c1c(C(=O)O)c[nH]c1C(=O)c1ccc(CCOCCOCCOCCOCCOCCO)cc1 ; "RA"]

### Step 1—(3R)-3-{4-bromo-2H,3H-pyrrolo[2,3-b]pyridin-1-yl}-1-(3-fluorophenyl)piperidine

To a stirred mixture of 1-phenyl-2,5,8,11,14,17-hexaoxanonadecan-19-ol (6.86 g, 18.4 m mol CAS #2615-15-8) in 2-methoxy-2-methylpropane (20 mL) were added 5,7-di-tert-butyl-3-phenyl-1,3lambda5-benzoxazol-3-ylium boron trifluoride fluoride (2.91 g, 7.37 mmol) and pyridine (0.58 g, 7.37 mmol) at rt under nitrogen atmosphere. The mixture was stirred for 20 min at rt under nitrogen atmosphere. The resulting mixture was filtered, and the filter cake was washed with 2-methoxy-2-methylpropane (20 mL). The filtrate was used directly. Next, to a stirred mixture of methyl 5-(4-bromobenzoyl)-4-(3,6-difluoro-2-methylphenyl)-1H-pyrrole-3-carboxylate (2 g, 4.6 mmol, Intermediate PR) and Photocatalyst (0.63 g, 0.69 mmol) in DMA (20 mL) were added the nickel catalyst (0.11 g, 0.23 mmol) and 2,3-dihydro-1H-isoindole-1,3-dione (6.78 mg, 0.046 mmol) at rt under nitrogen atmosphere. To the above mixture was added filtrate at rt. The vial was stirred at 1500 rpm stir rate and irradiated under 450 nm LED modules at 100% light intensity in a PennOC Integrated Photoreactor for 2 h. On completion, the residue was purified by reverse phase flash chromatography (Column: WelFlash™ C18-I, 20-40 um, 120 g; Eluent A: Water (plus 10 mmol/L NH₄HCO₃); Eluent B: ACN; Gradient: 25%-70% B in 25 min; Flow rate: 80 mL/min; Detector: 2 20/254 nm; desired fractions were collected at 50% B and concentrated under reduced pressure) to afford the title compound (330 mg, 10% yield) as a colorless oil. LC/MS (ESI, m/z): [(M−H)]⁺=620.2.

### Step 2—4-(3,6-Difluoro-2-methylphenyl)-5-[4-(17-hydroxy-3,6,9,12,15-pentaoxaheptadecan-1-yl)benzoyl]-1H-pyrrole-3-carboxylic acid

To a stirred solution of methyl 4-(3,6-difluoro-2-methylphenyl)-5-[4-(17-hydroxy-3,6,9,12,15-pentaoxaheptadecan-1-yl)benzoyl]-1H-pyrrole-3-carboxylate (330 mg, 0.533 mmol) in MeOH (5 m L) was added LiOH·H₂O (5 mL, 10 mmol) dropwise at rt under nitrogen atmosphere. The resulting mixture was then stirred for 3 h at 80° C. under nitrogen atmosphere. On completion, the mixture was cooled to rt and concentrated under reduced pressure. The mixture was acidified to pH 5 with cone. HCl. The resulting mixture was cooled to rt and diluted with water (20 mL), then extracted with EtOAc (3×10 mL). The combined organic layers were washed with brine (20 mL), and dried over anhydrous Na₂SO₄. After filtration, the filtrate was concentrated under reduced pressure to give the title compound (290 mg, 90% yield) as a yellow oil. LC/MS (ESI, m/z): [(M+H)]⁺=604.2.

### (3S)-3-(3-chlorophenyl)-3-{[4-(3,6-difluoro-2-methylphenyl)-5-[4-(17-oxo-3,6,9,12,15-pentaoxaheptadecan-1-yl)benzoyl]-1H-pyrrol-3-yl]formamido}propenamide (Intermediate RB)

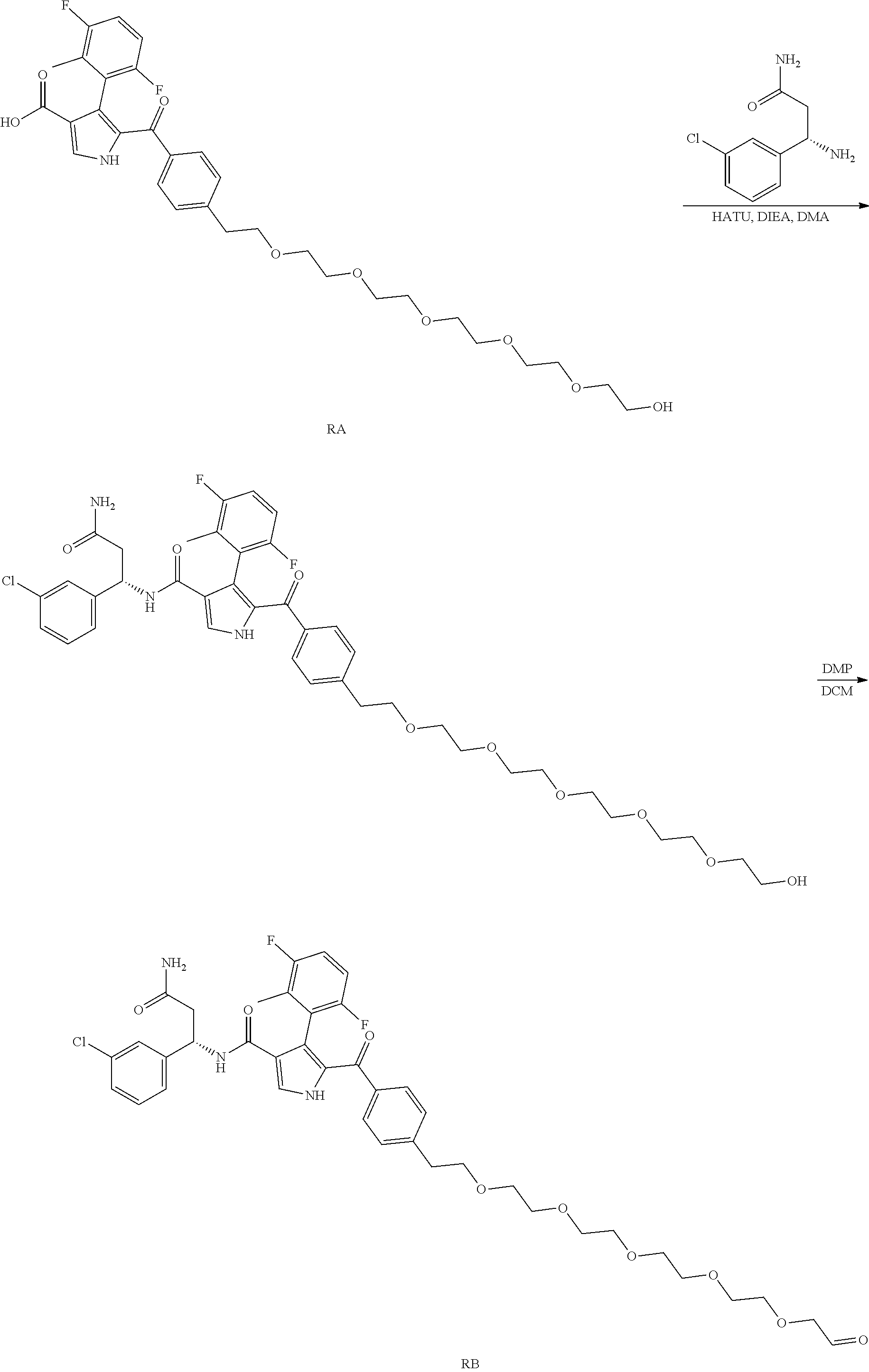

### Step 1—(3S)-3-(3-chlorophenyl)-3-{[4-(3,6-difluoro-2-methylphenyl)-5-[4-(17-hydroxy-3,6,9,12,15-pentaoxaheptadecan-1-yl)benzoyl]-1H-pyrrol-3-yl]formamido}propanamide

To a stirred solution of 4-(3,6-difluoro-2-methylphenyl)-5-[4-(17-hydroxy-3,6,9,12,15-pentaoxaheptadecan-1-yl)benzoyl]-1H-pyrrole-3-carboxylic acid (290 mg, 0.479 mmol. Intermediate RA) in DMA (10 mL) was added DIEA (0.25 mL, 1.44 mmol) in portions at rt under nitrogen atmosphere. To the above mixture was added (3S)-3-amino-3-(3-chlorophenyl)propanamide (142.68 mg, 0.718 mmol) in portions at rt. The resulting mixture was stirred for 2 h at rt under nitrogen atmosphere. On completion, the mixture was concentrated under reduced pressure. The residue was purified by reverse phase flash chromatography (Column: WelFlash™ C18-I, 20-40 um, 120 g; Eluent A: Water (plus 10 mmol/L NH₄CO₃); Eluent B: ACN; Gradient: 25%-70% B in 25 min; Flow rate: 80 mL/min; Detector: 220/254 nm; desired fractions were collected at 64% B and concentrated under reduced pressure) to afford the title compound (300 mg, 80% yield) as a brown oil. LC/MS (ESI, m/z): [(M−H)]⁺=784.3.

### Step 2—(3S)-3-(3-chlorophenyl)-3-{[4-(3,6-difluoro-2-methylphenyl)-5-[4-(17-oxo-3,6,9,12,15-pentaoxaheptadecan-1-yl)benzoyl]-1H-pyrrol-3-yl]formamido}propanamide

To a stirred solution of (3S)-3-(3-chlorophenyl)-3-{[4-(3,6-difluoro-2-methylphenyl)-5-[4-(17-hydroxy-3,6,9,12,15-pentaoxaheptadecan-1-yl)benzoyl]-1H-pyrrol-3-yl]formamido}propanamide (200 mg, 0.254 mmol) in DCM (3 mL) was added DMP (129.46 mg, 0.305 mmol) in portions at rt under nitrogen atmosphere. The resulting mixture was stirred for 2 h at rt under nitrogen atmosphere. On completion, the reaction was quenched by the addition of water (5 mL) at rt. The resulting mixture was concentrated under reduced pressure. The residue was purified by reverse phase flash chromatography (Column: WelFlash™ C18-I, 20-40 um, 120 g; Eluent A: Water (plus 10 mmol/L FA); Eluent B: ACN; Gradient: 25%-75% B in 25 min; Flow rate: 80 mL/min; Detector: 220/254 nm: desired fractions were collected at 47% B and concentrated under reduced pressure) to afford the title compound (120 mg, 60% yield) as a white solid. LC/MS (ESI, m/z): [(M+H)]⁺=784.4.

### 4-(3,6-Difluoro-2-methylphenyl)-5-[4-(10-hydroxydecyl)benzoyl]-1-methylpyrrole-3-carboxylic acid (Intermediate RC)

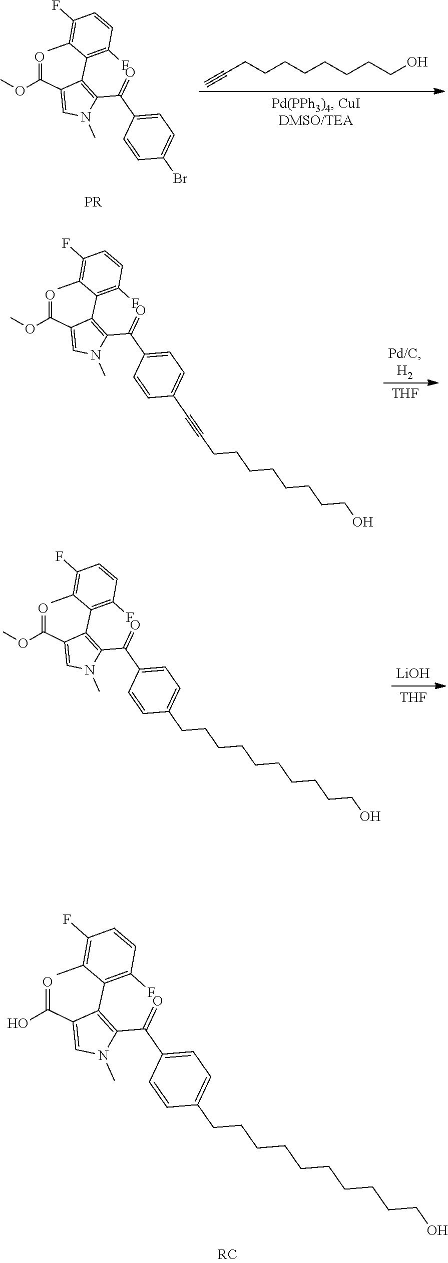

### Step 1—Methyl 4-(3,6-difluoro-2-methylphenyl)-5-[4-(10-hydroxydec-1-yn-1-yl)benzoyl]-1-methylpyrrole-3-carboxylate

To a stirred solution of methyl 5-(4-bromobenzoyl)-4-(3,6-difluoro-2-methylphenyl)-1-m ethylpyrrole-3-carboxylate (1.1 g, 2.5 mmol, Intermediate PR) and dec-9-yn-1-ol (0.76 g, 4.90 mmol) in DMSO (20 mL) were added TEA (5 mL) and CuI (0.09 g. 0.49 mmol) in portions at rt under nitrogen atmosphere. To the above mixture was added Pd(PPh₃)₄ (0.28 g, 0.25 mmol) in portions at rt. The resulting mixture was stirred for 2 h at 80° C. under nitrogen atmosphere. On completion, the mixture was cooled to rt and concentrated under reduced pressure. The residue was purified by reverse phase flash chromatography (Column: WelFlash™ C18-I, 20-40 um, 120 g; Eluent A: Water (plus 10 mmol/L NH4HCO₃): Eluent B: ACN; Gradient: 35%-90% B in 35 min: Flow rate: 80 mL/min; Detector: 220/254 nm: desired fractions were collected at 87% B and concentrated under reduced pressure) to afford the title compound (1.15 g, 90% yield) as a brown oil. LC/MS (ESI, m/z): [(M+H)]⁺=522.2.

### Step 2—4-(3,6-Difluoro-2-methylphenyl)-5-[4-(10-hydroxydecyl)benzoyl]-1-methylpyrrole-3-carboxylate

To a stirred solution of methyl 4-(3,6-difluoro-2-methylphenyl)-5-[4-(10-hydroxydec-1-yn-1-yl)benzoyl]-1-methylpyrrole-3-carboxylate (1.1 g, 2.1 mmol) in THF (20 mL) was added Pd/C (22.44 mg, 0.211 mmol) in portions at rt under nitrogen atmosphere. The resulting mixture was stirred for 2 h a t rt under hydrogen atmosphere. On completion, the mixture was filtered and the filtrated was washed with THF (20 mL) (2×20 mL). The organics were concentrated under reduced pressure to give the title compo und (1 g, 90% yield) as a brown oil. LC/MS (ESI, m/z): [(M+H)]⁺=526.3.

### Step 3—4-(3,6-Difluoro-2-methylphenyl)-5-[4-(10-hydroxydecyl)benzoyl]-1-methylpyrrole-3-carboxylic acid

To a stirred solution of methyl 4-(3,6-difluoro-2-methylphenyl)-5-[4-(10-hydroxydecyl)benzoyl]-1-methylpyrrole-3-carboxylate (1.1 g. 2.1 mmol) in MeOH (15 mL) was added LiOH·H₂O (15 mL, 30 mmol) in portions at rt under nitrogen atmosphere. The resulting mixture was stirred for 2 h at 80° C. under nitrogen atmosphere. On completion, the mixture was cooled to rt and concentrated under reduce d pressure. The mixture was acidified to pH 4 with cone. HCl. The resulting mixture was cooled to rt and diluted with water (30 mL), then extracted with EtOAc (3×50 mL). The combined organic layers were washed with brine (1×50 mL), and dried over anhydrous Na₂SO₄. After filtration, the filtrate was concentrated under reduced pressure to give the title compound (800 mg, 75% yield) as a brown oil. LC/MS (ESI, m/z): [(M+H)]⁺=511.3.

### 5-[4-(10-Bromodecyl)benzoyl]-N-[(1R)-1-(3-chlorophenyl)-2-methanesulfonylethyl]-4-(3,6-difluoro-2-methylphenyl)-1-methylpyrrole-3-carboxamide (Intermediate RD)

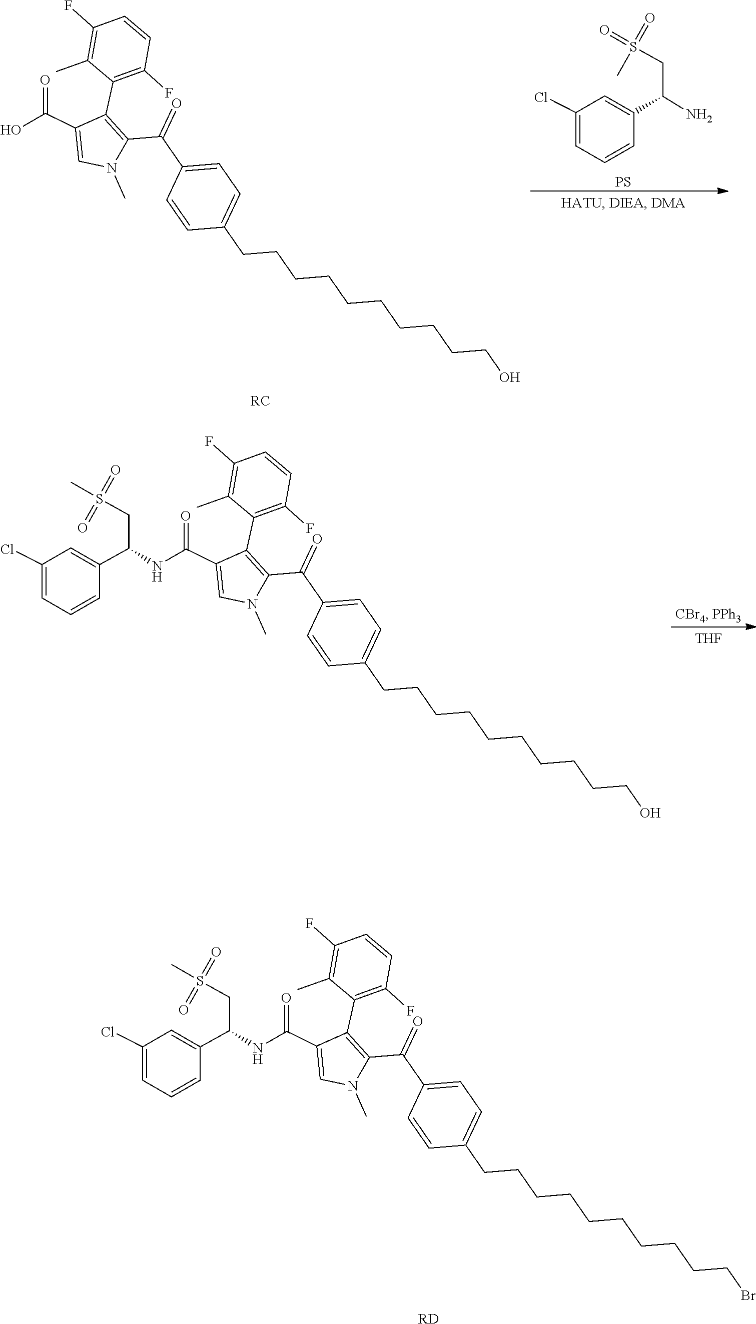

### Step 1—N-[(1R)-1-(3-chlorophenyl)-2-methanesulfonylethyl]-4-(3,6-difluoro-2-methylphenyl)-5-[4-(10-hydroxydecyl)benzoyl]-1-methylpyrrole-3-carboxamide

To a stirred solution of 4-(3,6-difluoro-2-methylphenyl)-5-[4-(10-hydroxydecyl)benzoyl]-1-methylpyrrole-3-carboxylic acid (300 mg, 0.586 mmol, Intermediate RC) and (1R)-1-(3-chlorophenyl)-2-methanesulfonylethanamine (205.57 mg, 0.879 mmol. Intermediate PS) in DMA (10 mL) was added TEA (0.24 mL, 1.758 mmol) dropwise at rt under nitrogen atmosphere. To the above mixture was added HATU (334.45 mg, 0.879 mmol) in portions at rt. The resulting mixture was stirred for 3 h at rt under nitrogen atmosphere. On completion, the mixture was purified by reverse phase flash chromatography (Column: WelFlash™ C18-I, 20-40 um, 120 g; Eluent A: Water (plus 10 mmol/L NH₄HCO₃); Eluent B: ACN; Gradient: 35%-90% B in 35 min; Flow rate: 80 mL/min; Detector: 220/254 nm; desired fractions were collected at 87% B and concentrated under reduced pressure) to afford the title compound (400 mg, 94% yield) as a colorless oil. LC/MS (ESI, m/z): [(M+H)]⁺=727.2.

### Step 2—5-[4-(10-Bromodecyl)benzoyl]-N-[(1R)-1-(3-chlorophenyl)-2-methanesulfonylethyl]-4-(3,6-difluoro-2-methylphenyl)-1-methylpyrrole-3-carboxamide

To a stirred solution of N-[(1R)-1-(3-chlorophenyl)-2-methanesulfonylethyl]-4-(3,6-difluoro-2-methylphenyl)-5-[4-(10-hydroxydecyl)benzoyl]-1-methylpyrrole-3-carboxamide (100 mg, 0.137 m mol) in DCM (5 mL) were added PPh₃ (43.28 mg, 0.164 mmol) and CBr₄ (68.40 mg, 0.206 mmol) in portions at rt under nitrogen atmosphere. The resulting mixture was stirred for 1 h at rt under nitrogen atmosphere. On completion, the mixture was purified by reverse phase flash chromatography (Column: WelFlash™ C18-I, 20-40 um, 120 g: Eluent A: Water (plus 10 mmol/L NH4HCO₃); Eluent B: ACN; Gradient: 35%-90% B in 35 min; Flow rate: 80 mL/min: Detector: 220/254 nm; desired fractions were collected at 87% B and concentrated under reduced pressure) to afford the title compound (80 mg, 74% yield) as a colorless oil. LC/MS (ESI, m/z): [(M+H)]=789.3, 791.3.

### 4-(3,6-Difluoro-2-methylphenyl)-5-[4-(12-hydroxydodecyl)benzoyl]-1-methylpyrrole-3-carboxylic acid (Intermediate RE)

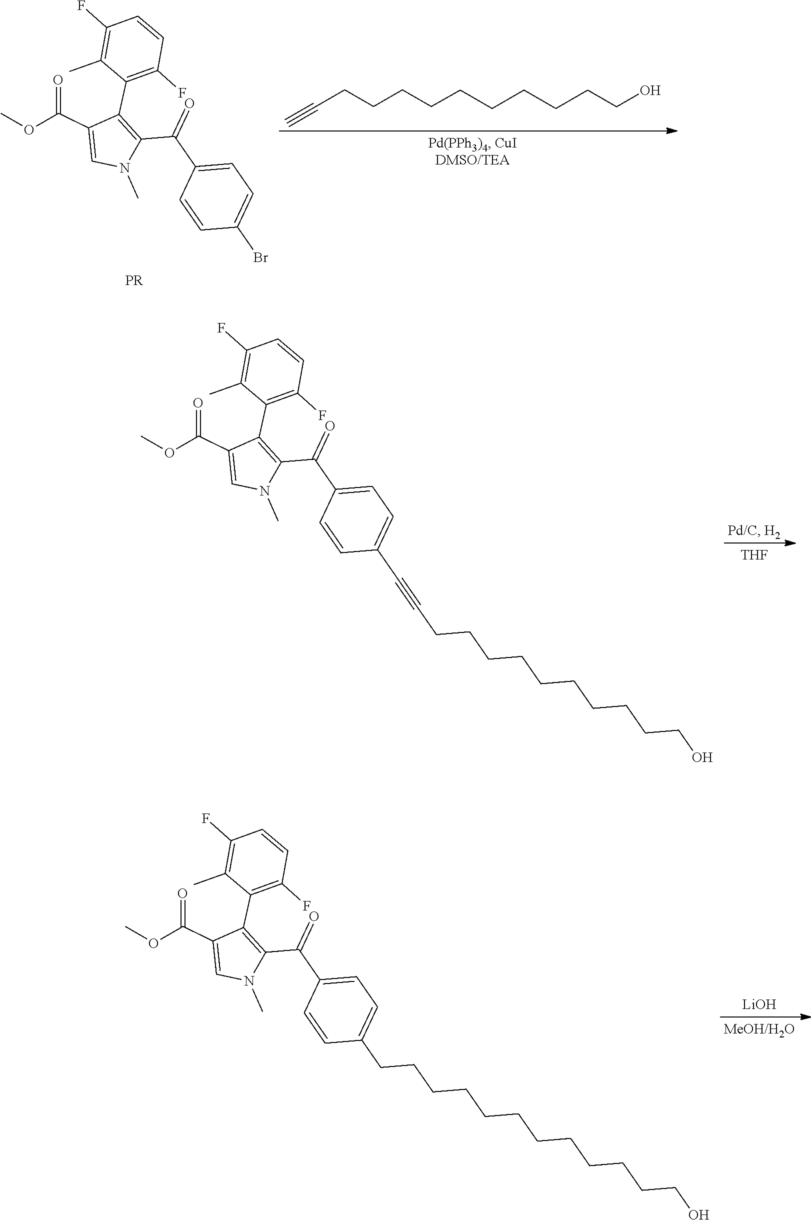

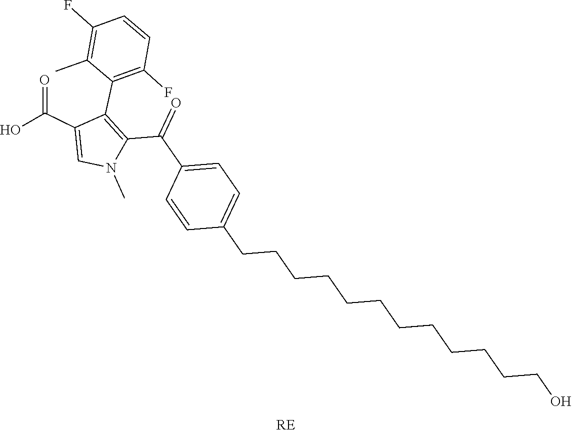 [SMILES: Cc1c(F)ccc(F)c1-c1c(C(=O)O)cn(C)c1C(=O)c1ccc(CCCCCCCCCCCCO)cc1]

### Step 1—4-(3,6-difluoro-2-methylphenyl)-5-[4-(12-hydroxydodec-1-yn-1-yl)benzoyl]-1-m ethylpyrrole-3-carboxylate

To a stirred mixture of methyl 5-(4-bromobenzoyl)-4-(3,6-difluoro-2-methylphenyl)-1-m ethylpyrrole-3-carboxylate (1 g, 2 mmol, Intermediate PR) and dodec-11-yn-1-ol (1.22 g, 6.69 mmol, CAS #18202-10-3), TEA (677.23 mg, 6.693 mmol) in DMSO (2 mL) was added Pd(PPh₃)₄ (257.79 mg, 0.223 mmol) at rt under nitrogen atmosphere. The resulting mixture was stirred for 2 h at 80° C. under nitrogen atmosphere. On completion, the mixture was cooled to rt and water was added to the reaction mixture (50 mL). The aqueous layer was extracted with EtOAc (3×30 mL). The resulting mixture was concentrated under reduced pressure. The residue was purified by silica gel column chromatography, eluted with CH₂Cl₂/MeOH (10:1), to afford the title compound (1 g, 82% yield) as a white solid. LC/MS (ESI, m/z): [(M+H)]+=550.3.

### Step 2—Methyl 4-(3,6-difluoro-2-methylphenyl)-5-[4-(12-hydroxydodecyl)benzoyl]-1-m ethylpyrrole-3-carboxylate

A mixture of methyl 4-(3,6-difluoro-2-methylphenyl)-5-[4-(12-hydroxydodec-1-yn-1-yl)benzoyl]-1-methylpyrrole-3-carboxylate (1 g, 2 mmol) and Pd/C (200 mg, 2 mmol) in THF (10 mL) was stirred for 30 min at rt under hydrogen atmosphere. On completion, the resulting mixture was filtered, and the filter cake was washed with EtOAc (3×10 mL). The filtrate was concentrated under reduced pressure. The residue was purified by reverse-phase flash chromatography (column, C18 silica gel; mobile phase. MeCN in Water (10 mmol/L NH₄HCO₃), 20% to 55% gradient in 25 min: detector, UV 254 nm) to afford the title compound (1 g, 99% yield) as a brown oil. LC/MS (ESI, m/z): [(M+H)]⁺=554.30. ¹H NMR (400 MHz, Chloroform-d) δ 7.62 (s, 1H), 7.48-7.35 (m, 2H), 7.00-6.85 (m, 2H), 6.66-6.55 (m, 2H), 4.14 (d, J=7.2 Hz, 1H), 4.00 (s, 3H), 3.70-3.62 (m, 5H), 2.50 (d, J=7.6 Hz, 2H), 1.88 (d, J=2.7 Hz, 3H). 1.49 (d, J=7.6 Hz, 6H), 1.35-1.25 (in. 14H).

### Step 3—4-(3,6-Difluoro-2-methylphenyl)-5-[4-(12-hydroxydodecyl)benzoyl]-1-methylpyrrole-3-carboxylic acid

A mixture of methyl 4-(3,6-difluoro-2-methylphenyl)-5-[4-(12-hydroxydodecyl)benzoyl]-1-methylpyrrole-3-carboxylate (1 g, 2 mmol) and LiOH·H₂O (10 mL, 20 mmol) in MeOH (10 mL) was stirred for 2 h at 90° C. under air atmosphere. On completion, the mixture was cooled to rt and acidified to pH 3 with cone. HCl. The aqueous layer was extracted with EtOAc (3×10 mL). The resulting mixture was concentrated under reduced pressure. The residue was purified by reverse-phase flash chromatography (column, C18 silica gel; mobile phase, MeCN in Water (10 mmol/L NH₄HCO₃), 30% to 50% gradient in 20 min; detector, UV 254 nm) to afford the title compound (900 mg, 92% yield) as a light brown solid. LC/MS (ESI, m/z): [(M+H)]⁺=540.3; ¹H NMR (400 MHz. DMSO-d₆) δ 11.98 (s, 1H), 7.92 (s, 1H), 7.31 (d, J=7.9 Hz, 2H), 6.97 (d, J=8.0 Hz, 2H), 6.70 (dd, J=24.0, 4.6 Hz, 2H), 4.31 (d, J=5.2 Hz, 1H), 3.37 (d, J=6.4 Hz, 5H), 2.46 (d, J=7.4 Hz, 2H), 1.87 (d, J=2.5 Hz, 3H), 1.43 (dd, J=13.0, 6.8 Hz. 4H), 1.26 (s, 14H), 1.20-1.12 (m, 2H).

### 5-[4-(12-Bromododecyl)benzoyl]-N-[(1R)-1-(3-chlorophenyl)-2-methanesulfonylethyl]-4-(3,6-difluoro-2-methylphenyl)-1-methylpyrrole-3-carboxamide (Intermediate RF)

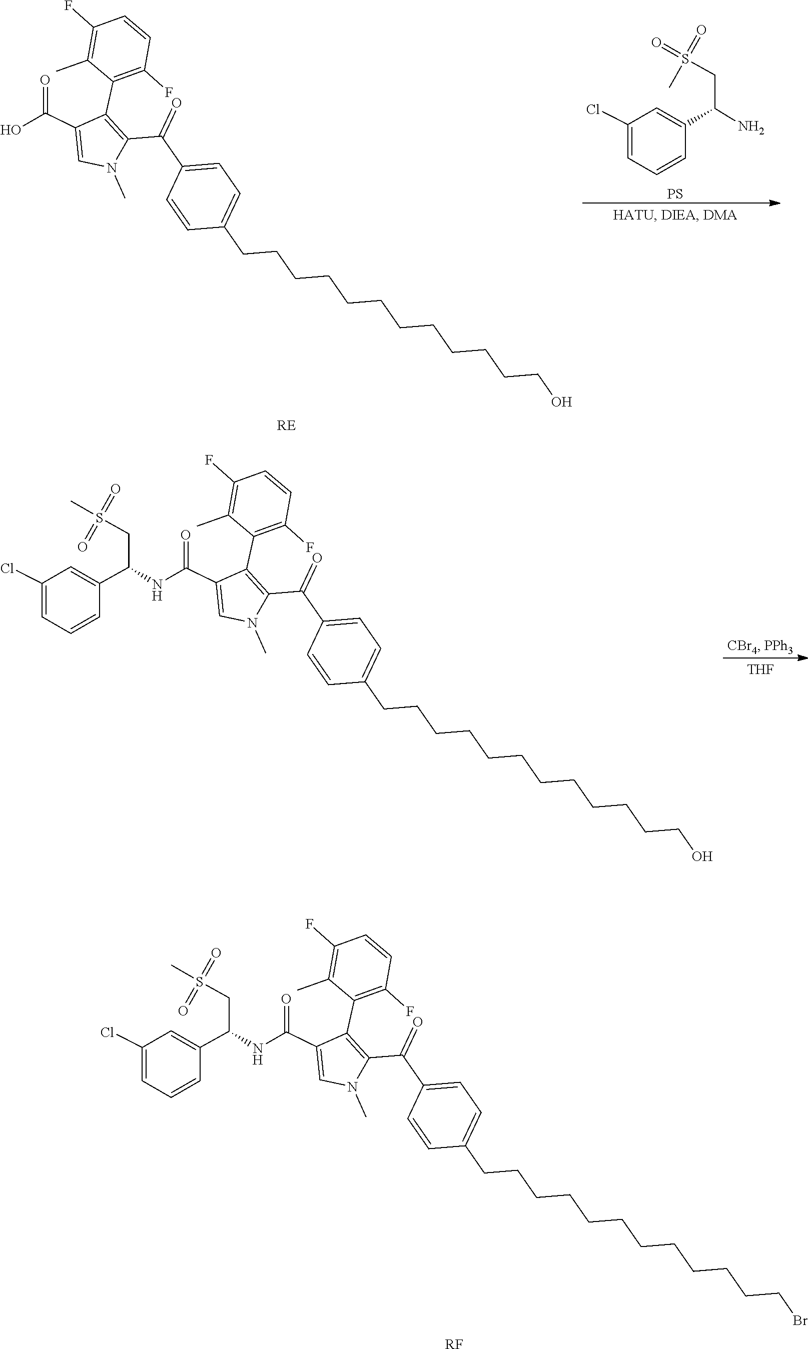

### Step 1—N-[(1R)-1-(3-chlorophenyl)-2-methanesulfonylethyl]-4-(3,6-difluoro-2-methylphenyl)-5-[4-(12-hydroxydodecyl)benzoyl]-1-metlhylpyrrole-3-carboxamide

To a stirred mixture of 4-(3,6-difluoro-2-methylphenyl)-5-[4-(12-hydroxydodecyl)benzo yl]-1-methylpyrrole-3-carboxylic acid (250 mg, 0.463 mmol, Intermediate RE) and HATU (264.22 mg, 0.695 mmol), DIEA (179.62 mg, 1.389 mmol) in DMA (10.00 mL, 107.5 mmol) was added (1R)-1-(3-chlorophenyl)-2-methanesulfonylethanamine (162.40 mg, 0.695 mmol, Intermediate PS) at rt under air atmosphere. The resulting mixture was stirred for 1 h at rt under air atmosphere. On completion, the mixture was purified by reverse-phase flash chromatography (column, C18 silica gel; mobile phase, MeCN in Water (10 mmol/L NH4HCO₃), 30% to 65% gradient in 20 min; detector, UV 254 nm) to afford the title compound (300 mg. 86% yield) as a colorless solid. LC/MS (ESI, m/z): [(M+H)]⁺=755.4: ¹H NMR (400 MHz, DMSO-d₆) δ 8.50 (dd, J=13.0, 8.4 Hz. 1H), 7.85 (d, J=7.4 Hz, 1H), 7.49-7.22 (m, 6H), 6.96 (d, J=7.8 Hz, 2H), 6.62-6.45 (m, 2H), 5.50-5.32 (m, 1H), 4.31 (dd, J=5.7, 4.6 Hz, 1H), 3.91 (s, 3H), 3.72-3.55 (m, 2H), 3.38 (dd, J=6.6, 5.2 Hz, 2H), 2.97 (d, J=9.4 Hz, 3H), 2.45 (d, J=7.4 Hz, 2H), 1.81 (dd, J=52.5, 2.5 Hz, 3H), 1.42 (d, J=7.2 Hz, 4H), 1.26 (s, 14H), 1.15 (s, 2H).

### Step 2—5-[4-(12-Bromododecyl)benzoyl]-N-[(1R)-1-(3-chlorophenyl)-2-methanesulfonylethyl]-4-(3,6-difluoro-2-methylphenyl)-1-methylpyrrole-3-carboxamide

To a stirred mixture of N-[(1R)-1-(3-chlorophenyl)-2-methanesulfonylethyl]-4-(3,6-difluoro-2-methylphenyl)-5-[4-(12-hydroxydodecyl)benzoyl]-1-methylpyrrole-3-carboxamide (100 mg, 0.132 mmol) and PPh₃ (52.09 mg, 0.198 mmol) in DCM (2 mL) was added CBr4 (65.85 mg, 0.198 mmol) at rt under air atmosphere. The resulting mixture was stirred for 4 h at rt under air atmosphere. On completion, the resulting mixture was concentrated under reduced pressure. The residue was purified by reverse-phase flash chromatography (column, C18 silica gel; mobile phase, MeCN in Water (10 mmol/L NH₄HCO₃), 25% to 50% gradient in 20 min: detector, UV 254 nm) to afford the title compound (80 mg, 74% yield) as a white solid. LC/MS (ESI, m/z): [(M+H)]⁺=818.3; ¹H NMR (400 MHz, DMSO-d₆) δ 8.50 (dd, J=13.0, 8.4 Hz, 1H), 7.85 (d, J=7.4 Hz, 1H), 7.49-7.23 (m, 6H), 6.96 (d, J=7.8 Hz, 2H), 6.62-6.42 (in, 2H), 5.52-5.33 (m, 1H), 3.91 (s, 3H), 3.72-3.55 (m, 2H), 3.52 (d, J=6.7 Hz, 2H), 2.97 (d, J=9.4 Hz, 3H), 2.45 (d, J=7.4 Hz, 2H), 1.90-1.71 (m, 5H), 1.48-1.11 (m, 20H).

### (1R,4R)-4-(prop-2-yn-1-yloxy)cyclohexan-1-ol (Intermediate RG)

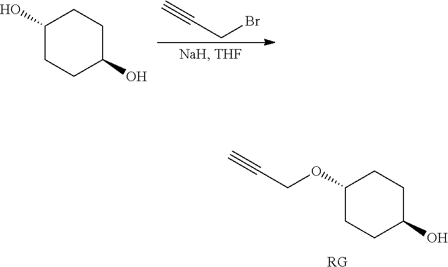

A solution of (1r,4r)-cyclohexane-1,4-diol (3 g, 30 mmol, CAS #6995-79-5) in DMF (60 g) was treated with NaH (1.2 g, 50 mmol) and the mixture was stirred for 30 min at 0° C. under nitrogen atmosphere. Next, propargyl bromide (3.6 g, 30 mmol) was added to the mixture in portions at 0° C. The resulting mixture was stirred for 16 h at rt under air atmosphere. On completion, the mixture was extracted with EtOAc (3×30 mL). The combined organic layers were washed with water (3×10 mL), and dried over anhydrous MgSO₄. After filtration, the filtrate was concentrated under reduced pressure. The residue was purified by silica gel column chromatography, eluted with PE/EA (1:1) to afford the title compound (560 mg, 14% yield) as a yellow oil. LC/MS (ESI, m/z): [(M+H)]⁺=154.2.

### 5-(4-Bromobenzoyl)-N-[(1R)-1-(3-chlorophenyl)-2-methanesulfonylethyl]-4-(3,6-difluoro-2-methylphenyl)-1-methylpyrrole-3-carboxamide (Intermediate RH)

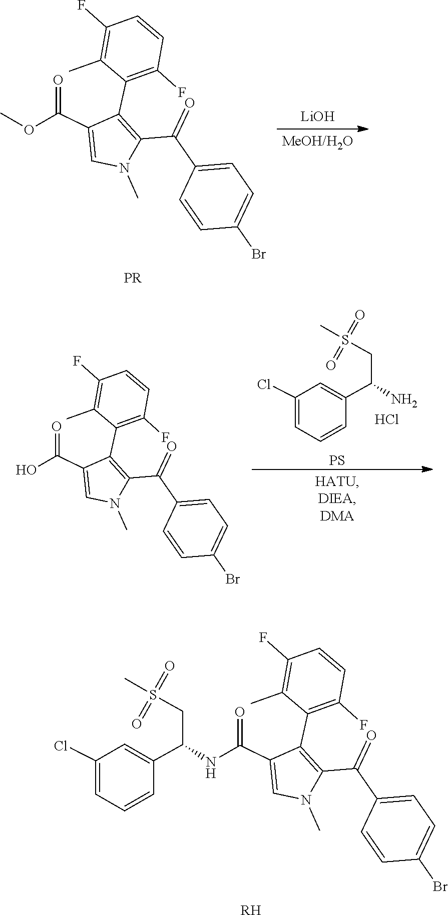

### Step 1—5-(4-Bromobenzoyl)-4-(3,6-difluoro-2-methylphenyl)-1-methylpyrrole-3-carboxylic acid

A mixture of methyl 5-(4-bromobenzoyl)-4-(3,6-difluoro-2-methylphenyl)-1-methylpyrrole-3-carboxylate (500 mg, 1.12 mmol, Intermediate PR) in MeOH (5 mL) was added a solution of 2 N Li OH·H₂O (5 mL) and then mixture was stirred for 2 h at 80° C. under air atmosphere. On completion, the mixture was cooled to rt and acidified to pH 3 with cone. HCl. The aqueous layer was extracted with EtOAc (3×10 mL). The combined organic layers were concentrated under reduced pressure. The residue was purified by reverse-phase flash chromatography (column, C18 silica gel: mobile phase, MeCN in Water (10 mmol/L NH₄HCO₃), 25% to 50% gradient in 30 min; detector, UV 254 nm) to afford the title compound (480 mg, 99% yield) as a white solid. LC/MS (ESI, m/z): [(M+H)]⁺=434.2.

### Step 2—5-(4-Bromobenzoyl)-N-[(1R)-1-(3-chlorophenyl)-2-methanesulfonylethyl]-4-(3,6-difluoro-2-methylphenyl)-1-methylpyrrole-3-carboxamide

To a stirred mixture of 5-(4-bromobenzoyl)-4-(3,6-difluoro-2-methylphenyl)-1-methylpyrrole-3-carboxylic acid (500 mg, 1.15 mmol) and HATU (656.73 mg, 1.727 mmol), DIEA (446.46 mg, 3, 453 mmol) in DMA (7 mL) was added (1R)-1-(3-chlorophenyl)-2-methanesulfonylethanamine (322.93 mg, 1.381 mmol, Intermediate PS) at rt under air atmosphere. The resulting mixture was stirred for 1 h at rt under air atmosphere. After completion, the mixture was purified by reverse-phase flash chromatography (column, C18 silica gel; mobile phase, MeCN in Water (10 mmol/L NH₄HCO₃). 30% to 50% gradient in 20 min; detector, UV 254 nm) to afford the title compound (500 mg, 67% yield) as a white solid. LC/MS (ESI, m/z): [(M+H)]⁺=649.9.

### N-[(1R)-1-(3-chlorophenyl)-2-methanesulfonylethyl]-4-(3,6-difluoro-2-methylphenyl)-1-methyl-5-[4-(3-{[(1s,4s)-4-iodocyclohexyl]oxy}prop-1-yn-1-yl)benzoyl]pyrrole-3-carboxamide (Intermediate RI)

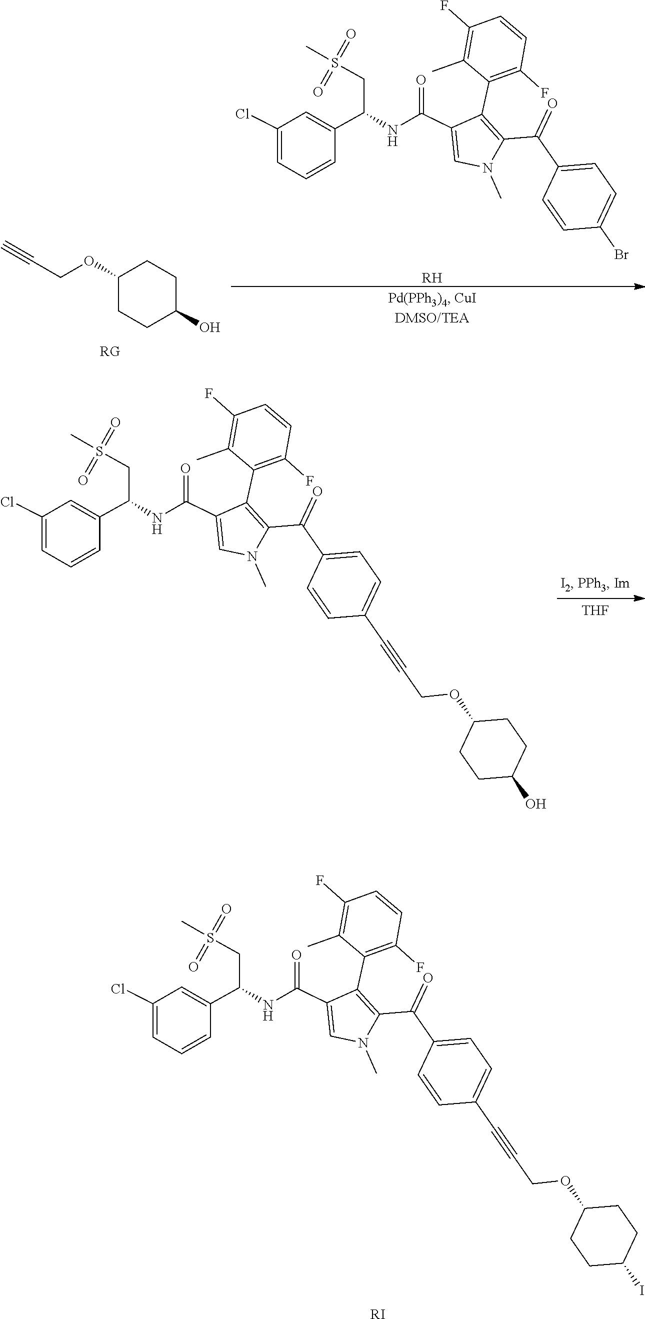

### Step 1— N-[(1R)-1-(3-chlorophenyl)-2-methanesulfonylethyl]-4-(3,6-difluoro-2-methylphenyl)-1-methyl-5-[4-(3-{[(1r,4r)-4-hydroxycyclohexyl]oxy}prop-1-yn-1-yl)benzoyl]pyrrole-3-carboxamide

To a stirred solution of (1r,4r)-4-(prop-2-yn-1-yloxy)cyclohexan-1-ol (500 mg, 3.24 mmol, Intermediate RG) and 5-(4-bromobenzoyl)-N-[(1R)-1-(3-chlorophenyl)-2-methanesulfonylethyl]-4-(3,6-difluoro-2-methylphenyl)-1-methylpyrrole-3-carboxamide (2 g. 3 mmol, Intermediate RH) in DMSO (10 mL) and TEA (3 mL) were added Pd(PPh₃)₄ (380 mg, 0.329 mmol) and CuI (125 mg, 0.656 mmol) in portions at rt under air atmosphere. The resulting mixture was stirred for 2 h at 80° C. under air atmosphere. On completion, the mixture was cooled to rt and concentrated under reduced pressure. The residue was purified by reverse phase Flash chromatography (Column: WelFlash™ C18-I, 20-40 um, 330 g; Eluent A: Water (plus 10 mmol/L NH4HCO3); Eluent B: ACN; Gradient: 45%-65% B in 25 min; Flow rate: 80 mL/min; Detector: 220/254 nm; desired fractions were collected at 56% B and concentrated under reduced pressure) to afford the title compound (870 mg, 37% yield) as a brown solid. LC/MS (ESI, m/z): [(M+H)]⁻=723.2.

### Step 2—N-[(1R)-1-(3-chlorophenyl)-2-methanesulfonylethyl]-4-(3,6-difluoro-2-methylphenyl)-1-methyl-5-[4-(3-{[(1s,4s)-4-iodocyclohexyl]oxy}prop-1-yn-1-yl)benzoyl]pyrrole-3-carboxamide

To a stirred solution of N-[(1R)-1-(3-chlorophenyl)-2-methanesulfonylethyl]-4-(3,6-difluoro-2-methylphenyl)-1-methyl-5-[4-(3-{1[(1r,4r)-4-hydroxycyclohexyl]oxy}prop-1-yn-1-yl)benzoyl]pyrrole-3-carboxamide (500 mg, 0.691 mmol) and 12 (265 mg, 1.044 mmol) in THF were added imidazole (100 mg, 1.47 mmol) and PPh₃ (360 mg, 1.37 mmol) in portions at rt under air atmosphere. The resulting mixture was stirred for 2 h at rt under air atmosphere. On completion, the mixture was concentrated under reduced pressure. The residue was purified by reverse phase Flash chromatography (Column: WelFlash™ C18-I, 20-40 um, 330 g: Eluent A: Water (plus 10 mmol/L NH₄HCO₃); Eluent B: ACN: Gradient: 25%-55% B in 25 min; Flow rate: 80 mL/min; Detector: 220/254 nm; desired fractions were collected at 50% B and concentrated under reduced pressure) to afford the title compound (380 mg, 66% yield) as a yellow solid. LC/MS (ESI, m/z): [(M+H)]⁺=833.1.

### N-[(1R)-1-(3-chlorophenyl)-2-methanesulfonylethyl]-4-(3,6-difluoro-2-methylphenyl)-1-methyl-5-{4-[3-(4-oxopiperidin-1-yl)prop-1-yn-1-yl]benzoyl}pyrrole-3-carboxamide (Intermediate RJ)

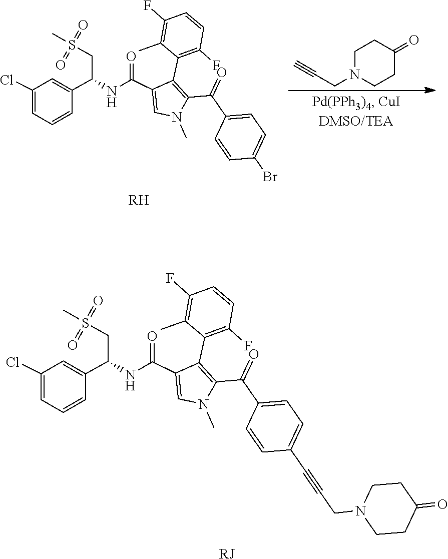

To a stirred solution of 5-(4-bromobenzoyl)-N-[(1R)-1-(3-chlorophenyl)-2-methanesulfonylethyl]-4-(3,6-difluoro-2-methylphenyl)-1-methylpyrrole-3-carboxamide (100 mg, 0.154 mmol, Intermediate RH) and 1-(prop-2-yn-1-yl)piperidin-4-one (65 mg, 0.47 mmol, CAS #857190-11-5) in DMSO (3 mL) and TEA (1 mL) were added CuI (6 mg, 0.03 mmol) and Pd(PPh₃)₄ (20 mg, 0.017 mmol) in portions at rt under nitrogen atmosphere. The resulting mixture was stirred for 2 h at 80° C. under nitrogen atmosphere. On completion, the mixture was cooled to rt and concentrated under reduced pressure. The residue was purified by reverse phase Flash chromatography (Column: WelFlash™ C18-I, 20-40 um, 120 g; Eluent A: Water (plus 10 mmol/L NH₄HCO₃); Eluent B: ACN; Gradient: 40%-60% B in 25 min; Flow rate: 60 mL/min; Detector: 220/254 nm; desired fractions were collected at 56% B and concentrated under reduced pressure) to afford the title compound (100 mg, 83% yield) as a yellow solid. LC/MS (ESI, m/z): [(M+H)]⁻=706.2.

### 6-[(1R,5S,6s)-3-(2-bromopyridin-4-yl)-3-azabicyclo[3.1.1]heptan-6-yl]hexan-1-ol (Intermediate RK) and 6-((1R,5S,6r)-3-(2-bromopyridin-4-yl)-3-azabicyclo[3.1.1]heptan-6-yl)hexan-1-ol (Intermediate RL)

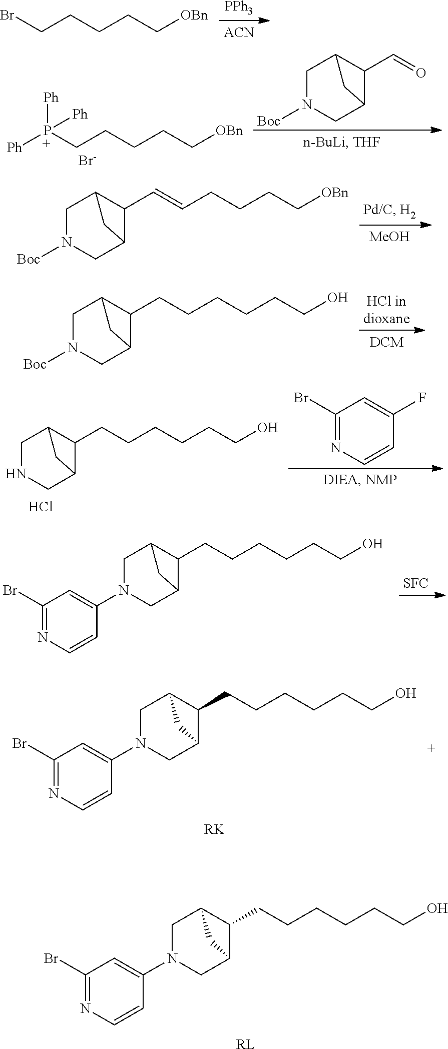

### Step 1—[5-(Benzyloxy)pentyl]triphenylphosphanium

To a stirred solution of {[(5-bromopentyl)oxy]methyl}benzene (10 g, 40 mmol, CAS #1014-93-3) in ACN (150 mL) was added PPh₃ (30.60 g, 116.7 mmol) in portions at rt under air atmosphere. The resulting mixture was stirred for 16 h at 80° C. under air atmosphere. On completion, the mixture was cooled to rt and was purified by silica gel column chromatography, eluted with EA/MEOH (5:1), to afford the title compound (16 g, 94% yield) as a colorless oil. LC/MS (ESI, m/z): [(M+H)]⁺=439.2.

### Step 2—Tert-butyl 6-[6-(benzyloxy)hex-1-en-1-yl]-3-azabicyclo[3.1.1]heptane-3-carboxylate

To a stirred solution of [5-(benzyloxy)pentyl]triphenylphosphanium bromide (10 g, 20 mmol) in THF (150 mL) was added butyllithium (1.85 g. 28.9 mmol) dropwise at −78° C. under nitrogen atmosphere. The resulting mixture was stirred for 1 h at −60° C. under air atmosphere. To the above mixture was added tert-butyl 6-formyl-3-azabicyclo[3.1.1]heptane-3-carboxylate (4.34 g, 19.3 mmol, CAS #1818847-75-7) in portions at −78° C. The resulting mixture was stirred for additional 1 h at −60° C. On completion, the mixture was extracted with EtOAc (3×300 mL). The combined organic layers were dried over anhydrous Na₂SO₄. After filtration, the filtrate was concentrated under reduced pressure. The residue was purified by silica gel column chromatography, eluted with PE/EA (5:1), to afford the title compound (5.6 g, 76% yield) as a colorless oil. LC/MS (ESI, m/z): [(M+H−56)]⁺=330.2.

### Step 3—Tert-butyl 6-(6-hydroxyhexyl)-3-azabicyclo[3.1.1]heptane-3-carboxylate

To a solution of tert-butyl 6-[6-(benzyloxy)hexylidene]-3-azabicyclo[3.1.1]heptane-3-carboxylate (1.8 g, 4.7 mmol) in 50 mL MeOH was added Pd/C (10 wt %, 1 g) in a pressure tank. The mixture was hydrogenated at rt under 30 psi of hydrogen pressure for 1 h. On completion, the mixture was filtered through a Celite pad and concentrated under reduced pressure to give the title compound (1.3 g, 94%) as a colorless oil. LC/MS (ESI, m/z): [(M+H−56)]⁺=242.1.

### Step 4—6-{3-Azabicyclo[3.1.1]heptan-6-yl}hexan-1-ol

To a stirred solution of tert-butyl 6-(6-hydroxyhexyl)-3-azabicyclo[3.1.1]heptane-3-carboxylate (3.4 g, 11 mmol) in DCM (15 mL) was added HCl (gas) in 1,4-dioxane (15 mL) dropwise at rt under air atmosphere. The resulting mixture was stirred for 30 min at rt under air atmosphere. On completion, the mixture was concentrated under reduced pressure to give the title compound (3 g) as a colorless oil. LC/MS (ESI, m/z): [(M+H)]⁺=198.2.

### Step 5—6-[3-(2-bromopyridin-4-yl)-3-azabicyclo[3.1.1]heptan-6-yl]hexan-1-ol

To a stirred mixture of 6-{3-azabicyclo[3.1.1]heptan-6-yl}hexan-1-ol hydrochloride (1.7 g, 7.3 mmol) and DIEA (1.88 g, 14.5 mmol) in NMP (20 mL) was added 2-bromo-4-fluoropyridine (1.28 g, 7.27 mmol) in portions at rt under air atmosphere. The resulting mixture was stirred for 1 h at 100° C. under air atmosphere. On completion, the mixture was cooled to rt and was purified by reverse-phase flash chromatography (column. C18 silica gel; mobile phase. MeCN in Water (10 mmol/L NH4HCO3), 10% to 50% gradient in 50 min; detector, UV 254 nm) to give the title compound (1.3 g, 51% yield) as a colorless oil. LC/MS (ESI, m/z): [(M+H)]⁺=353.1.

### Step 6—6-((1R,5S,6s)-3-(2-bromopyridin-4-yl)-3-azabicyclo[3.1.1]heptan-6-yl)hexan-1-ol

6-[3-(2-bromopyridin-4-yl)-3-azabicyclo[3.1.1]heptan-6-yl]hexan-1-ol (3.2 g, 9.1 mmol) was separated by HPLC (Column: CHIRALPAK IH 3*25 cm, 5 μm; Mobile Phase A: CO2, Mobile Phase B: MEOH (0.1% 2M NH₃-MEOH); Flow rate: 100 mL/min; Gradient: isocratic 30% B; RT1 (min): 4: RT2 (min): 6; Sample Solvent: MEOH; Injection Volume: 3 mL; Number Of Runs: 30) to afford the first eluting isomer 6-((1R,5S,6s)-3-(2-bromopyridin-4-yl)-3-azabicyclo[3.1.1]heptan-6-yl)hexan-1-ol (1.4 g, 44%, ¹H NMR (400 MHz. Chloroform-d) δ 7.97 (d, J=5.9 Hz, 1H), 6.69 (d, J=2.4 Hz, 1H), 6.55-6.45 (m, 1H), 3.62 (t, J=6.5 Hz, 2H), 3.51-3.32 (m, 4H), 2.52 (t, J=6.2 Hz, 2H), 2.23 (p, J=6.6 Hz, 1H), 2.08 (dd, J=9.8, 5.6 Hz, 1H), 1.88 (s, 2H), 1.55 (t, J=7.1 Hz, 2H), 1.43 (d, J=9.5 Hz, 1H), 1.38-1.19 (m, 4H), 1.24-1.15 (m, 1H), 1.15 (s, 2H)) and the second eluting isomer 6-((1R,5S,6r)-3-(2-bromopyridin-4-yl)-3-azabicyclo[3.1.1]heptan-6-yl)hexan-1-ol (1.4 g, 44%, ¹H NMR (400 MHz, Chloroform-d) δ 7.94 (d, J=6.0 Hz, 1H), 6.66 (d, J=2.4 Hz, 1H), 6.46 (dd, J=6.0, 2.4 Hz, 1H), 3.65 (t, J=6.6 Hz, 2H), 3.57-3.42 (m, 4H), 2.42 (dt, J=10.4, 6.3 Hz, 1H), 2.29 (d, J=6.2 Hz, 2H), 2.14 (s, 1H), 1.72-1.55 (m, 4H), 1.37 (dt, J=15.7, 6.8 Hz, 8H) as a colorless oils. LC/MS (ESI, m/z): [(M+H)]⁺=353.1, 355.1 for both.

### 1R,5S,6s)-3-(2-bromopyridin-4-yl)-6-[6-(oxan-2-yloxy)hexyl]-3-azabicyclo[3.1.1]heptane (Intermediate RM)

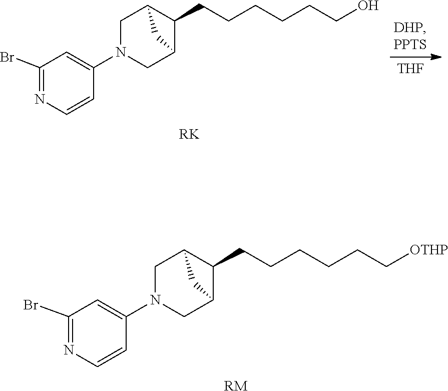

To a stirred solution of 6-[(1R,5S,6s)-3-(2-bromopyridin-4-yl)-3-azabicyclo[3.1.1]heptan-6-yl]hexan-1-ol (2 g, 6 mmol, Intermediate RK) in THF (15 mL) were added PPTS (0.28 g, 1.13 mmol) and DHP (4.76 g, 56.6 mmol) at rt. The resulting mixture was stirred for additional 6 h at 50° C. On completion, the mixture was purified by silica gel column chromatography, eluted with PE/EA (3:1), to afford the title compound (2.18 g, 88% yield) as a white oil. LC/MS (ESI, m/z): [(M+H)]⁺=437.2, LC/MS (ESI, m/z): [(M+H+2)]⁺=439.2.

### Methyl 3-[(3aR,7aR)-octahydropyrrolo[3,2-b]pyridin-4-yl]-5-fluorobenzoate (Intermediate RN)

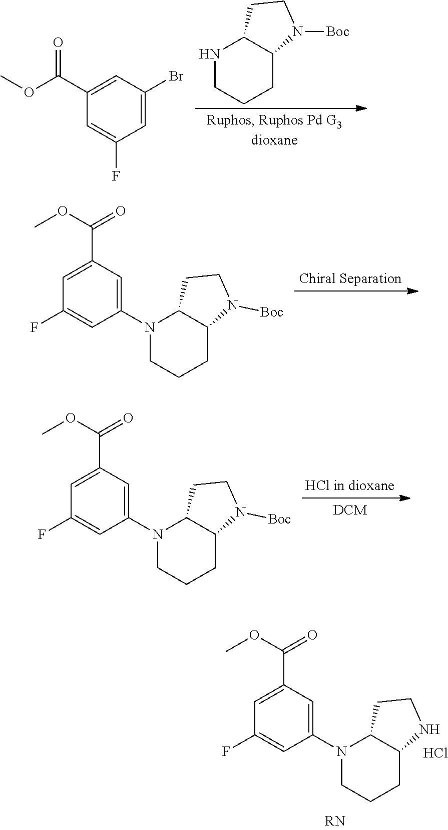

### Step 1—Tert-butyl (3aR,7aR)-4-[3-fluoro-5-(methoxycarbonyl)phenyl]-hexahydro-2H-pyrrolo[3,2-b]pyridine-1-carboxylate

To a stirred solution of methyl 3-bromo-5-fluorobenzoate (2 g, 9 mmol) and tert-butyl (3aR,7aR)-octahydropyrrolo[3,2-b]pyridine-1-carboxylate (1.94 g, 8.58 mmol, CAS #1821737-93-2) in 1,4-dioxane (20 mL) were added Cs₂CO₃ (5.59 g, 17.2 mmol) and {1,3-bis[2,6-bis(pentan-3-yl)phenyl]-4,5-dichloro-2,3-dihydro-1H-imidazol-2-yl}dichloro(2-methyl-llambda4-pyridin-1-yl)palladium (0.72 g, 0.86 mmol) at rt. The resulting mixture was stirred for 2 h at 100° C. under nitrogen atmosphere. On completion, the mixture was cooled to rt and concentrated under reduced pressure. The residue was purified by silica gel column chromatography, eluted with PE/EA (5:1), to afford the title compound (3.1 g, 95% yield) as a yellow oil. LC/MS (ESI, m/z): [(M+H)]⁺=379.2.

### Step 2—Tert-butyl (3aR,7aR)-4-(3-fluoro-5-(methoxycarbonyl)phenyl)octahydro-1H-pyrrolo[3,2-b]pyridine-1-carboxylate

Tert-butyl (3aR,7aR)-4-[3-fluoro-5-(methoxycarbonyl)phenyl]-hexahydro-2H-pyrrolo[3,2-b]pyridine-1-carboxylate (4 g. 10 mmol) was separated by HPLC (Column: CHIRALCEL OJ-H, 30 mm*250 mm; Mobile Phase A: Hex (0.5% DEA)-HPLC, Mobile Phase B: MeOH:EtOH=1:1-HPLC: Flow rate: 100 mL/min; Gradient: isocratic 2% B: Wave Length: 220 nm: RTI (min): 10.1: RT2 (min): 13.5; Sample Solvent: EtOH; Injection Volume: 10 mL) to afford the first eluting isomer tert-butyl (3aR,7aR)-4-(3-fluoro-5-(methoxycarbonyl)phenyl)octahydro-1H-pyrrolo[3,2-b]pyridine-1-carboxylate (1.5 g, 38% yield) as a colorless oil. LC/MS (ESI, m/z): [(M+H)]⁺=379.3. Absolute stereochemistry assigned arbitrarily.

### Step 3—Methyl 3-fluoro-5-((3aR,7aR)-octahydro-4H-pyrrolo[3,2-b]pyridin-4-yl)benzoate

To a stirred solution of tert-butyl (3aR,7aR)-4-[3-fluoro-5-(methoxycarbonyl)phenyl]-hexahydro-2H-pyrrolo[3,2-b]pyridine-1-carboxylate (3.1 g, 8.2 mmol) in DCM (30 mL) was added HCl (gas) in 1,4-dioxane (30 mL, 120 mmol) dropwise at rt. The resulting mixture was stirred for 1 h at rt under nitrogen atmosphere. On completion, the resulting mixture was concentrated under reduced pressure. The residue was purified by trituration with Et₂O (50 mL) to afford the title compound (2.5 g, 97%) as a yellow oil. LC/MS (ESI, m/z): [(M+H−56)]⁺=279.2.

### {3-[(3aR,7aR)-1-{4-[(1R,5S,6s)-6-(6-oxohexyl)-3-azabicyclo[3.1.1]heptan-3-yl]pyridin-2-yl}-hexahydro-2H-pyrrolo[3,2-b]pyridin-4-yl]-5-fluorophenyl}methyl carbamate (Intermediate RO)

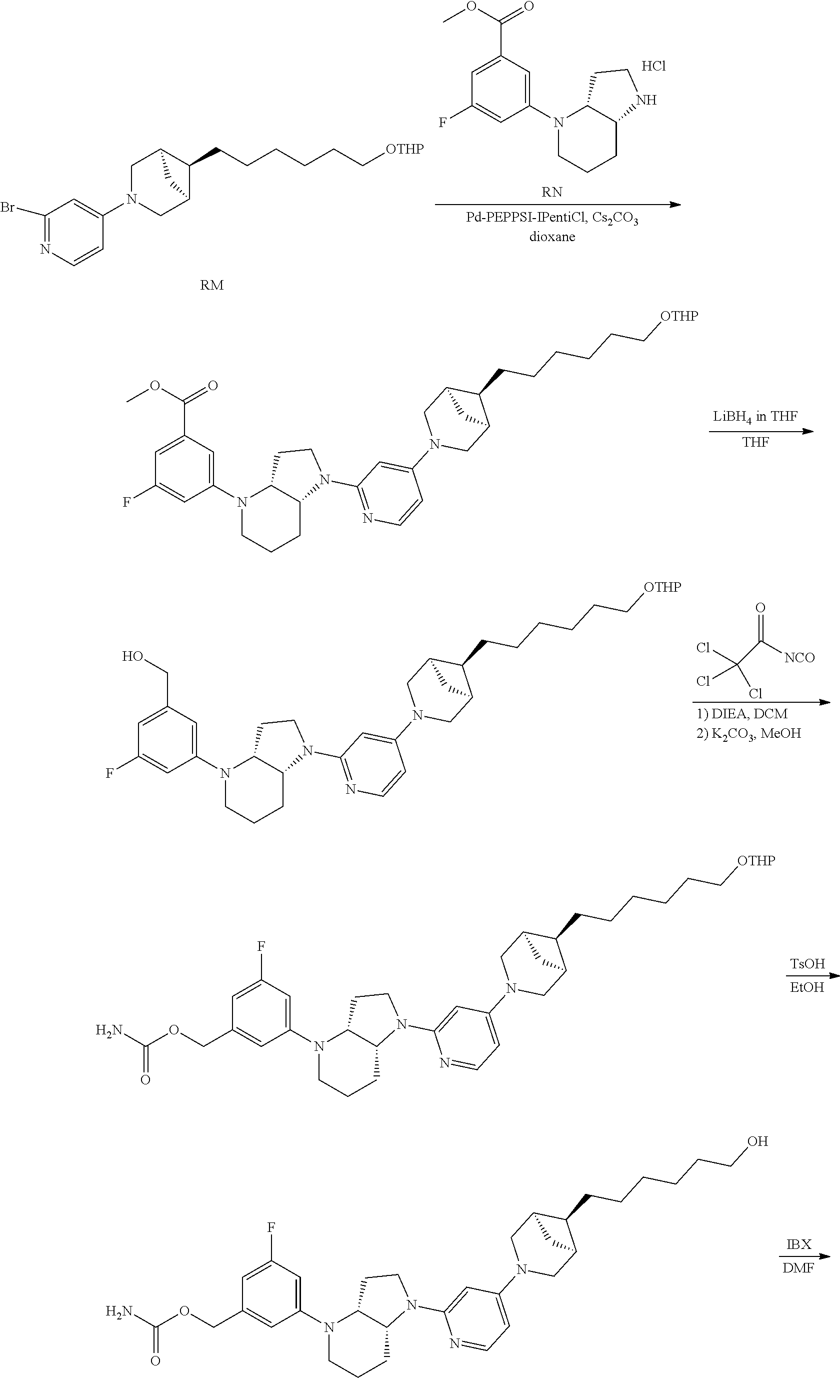

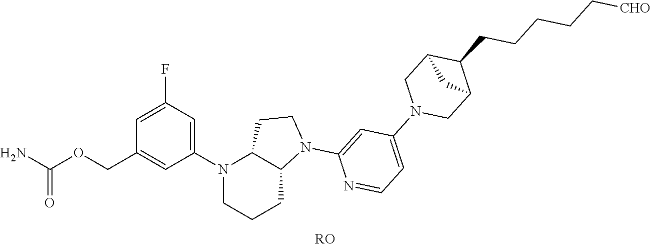 [SMILES: NC(=O)OCc1cc(F)cc(N2CCC[C@@H]3[C@H]2CCN3c2cc(N3C[C@H]4C[C@@H](C3)[C@H]4CCCCCC=O)ccn2)c1]

### Step 1—Methyl 3-[(3aR,7aR)-1-{4-[(1R,5S,6s)-6-[6-(oxan-2-yloxy)hexyl]-3-azabicyclo[3.1.1]heptan-3-yl]pyridin-2-yl}-hexahydro-2H-pyrrolo[3,2-b]pyridin-4-yl]-5-fluorobenzoate

To a stirred solution of (1R,5S,6s)-3-(2-bromopyridin-4-yl)-6-[6-(oxan-2-yloxy)hexyl]-3-azabicyclo[3.1.1]heptane (1 g, 2 mmol, Intermediate RM) in dioxane (10 mL) were added methyl 3-[(3aR,7aR)-octahydropyrrolo[3,2-b]pyridin-4-yl]-5-fluorobenzoate (0.64 g, 2.3 mmol, Intermediate RN), Cs₂CO₃ (2.23 g, 6.86 mmol) and Pd-PEPPSI-IPentCl₂ (0.19 g, 0.23 mmol) at rt. The resulting mixture was stirred for additional 2 h at 100° C. On completion, the mixture was cooled to rt and the mixture was purified by silica gel column chromatography, eluted with CH₂Cl₂/MeOH (10:1), to afford the title compound (590 mg, 41% yield) as a yellow solid. LC/MS (ESI, m/z): [(M+H)]⁺=635.4.

### Step 2—{3-[(3aR,7aR)-1-{4-[(1R,5S6s)-6-[6-(oxan-2-yloxy)hexyl]-3-azabicyclo[3.1.1]heptan-3-yl]pyridin-2-yl}-hexahydro-2H-pyrrolo[3,2-b]pyridin-4-yl]-5-fluorophenyl}methanol

To a stirred solution of methyl 3-[(3aR,7aR)-1-{4-[(1R,5S,6s)-6-[6-(oxan-2-yloxy)hexyl]-3-azabicyclo[3.1.1]heptan-3-yl]pyridin-2-yl}-hexahydro-2H-pyrrolo[3,2-b]pyridin-4-yl]-5-fluorobenzoate (0.58 g, 0.91 mmol) in THF (8 mL) was added LiBH₄ (0.10 g, 4.6 mmol) at rt. The resulting mixture was stirred for additional 12 h at 50° C. On completion, the mixture was cooled to rt and diluted with water (30 mL). The resulting mixture was extracted with EA (3×10 mL), and dried over anhydrous Na₂SO₄. After filtration, the filtrate was concentrated under reduced pressure to afford the title compound (471 mg) as a yellow solid. LC/MS (ESI, m/z): [(M+H)]⁺=607.4.

### Step 3—{3-[(3aR,7aR)-1-{4-[(1R,5S,6s)-6-[6-(oxan-2-yloxy)hexyl]-3-azabicyclo[3.1.1]heptan-3-yl]pyridin-2-yl}-hexahydro-2H-pyrrolo[3,2-b]pyridin-4-yl]-5-fluorophenyl}methyl carbamate

To a stirred solution of {3-[(3aR,7aR)-1-{4-[(1R,5S,6s)-6-[6-(oxan-2-yloxy)hexyl]-3-azabicyclo[3.1.1]heptan-3-yl]pyridin-2-yl}-hexahydro-2H-pyrrolo[3,2-b]pyridin-4-yl]-5-fluorophenyl}methanol (631 mg, 1.04 mmol) in DCM (5 mL) were added DIEA (0.36 ml, 2.1 mmol) and trichloroethanecarbonyl isocyanate (391.79 mg, 2.080 mmol) at rt. The resulting mixture was stirred 1 h at rt. Then the mixture was concentrated under reduced pressure. The residue was dissolved in MeOH (5 mL). To the above mixture was added K₂CO₃ (431.13 mg, 3.120 mmol) at rt. The resulting mixture was stirred for an additional 30 min at rt. On completion, the mixture was concentrated under reduced pressure and was diluted with water (30 mL). The resulting mixture was extracted with EA (3×10 mL), and dried over anhydrous Na₂SO₄. After filtration, the filtrate was concentrated under reduced pressure to afford the title compound (549 mg) as a yellow oil. LC/MS (ESI, m/z): [(M+H)]⁺=650.6.

### Step 4—{3-[(3aR,7aR)-1-{4-[(1R,5S,6s)-6-(6-hydroxyhexyl)-3-azabicyclo[3.1.1]heptan-3-yl]pyridin-2-yl}-hexahydro-2H-pyrrolo[3,2-b]pyridin-4-yl]-5-fluorophenyl}methyl carbamate

To a stirred solution of {3-[(3aR,7aR)-1-{4-[(1R,5S,6s)-6-[6-(oxan-2-yloxy)hexyl]-3-azabicyclo[3.1.1]heptan-3-yl]pyridin-2-yl}-hexahydro-2H-pyrrolo[3,2-b]pyridin-4-yl]-5-fluorophenyl}methyl carbamate (524 mg, 0.806 mmol) in EtOH (5 mL) were added TₛOH (138.85 mg, 0.806 mmol) at rt. The resulting mixture was stirred for 1 h at 80° C. On completion, the mixture was purified by reverse phase flash chromatography (Column: WelFlash™ C18-I, 20-40 um, 120 g; Eluent A: Water (plus 10 mmol/L NH₄CO₃): Eluent B: MeOH; Gradient: 30%-70% B in 40 min; Flow rate: 60 mL/min; Detector: 220/254 nm; desired fractions were collected at 60% B and concentrated under reduced pressure) to afford the title compound (243 mg, 53% yield) as a white solid. LC/MS (ESI, m/z): [(M+H)]⁻=566.4.

### Step 5—{3-[(3aR,7aR)-1-{4-[(1R,5S,6s)-6-(6-oxohexyl)-3-azabicyclo[3.1.1]heptan-3-yl]pyridin-2-yl}-hexahydro-2H-pyrrolo[3,2-b]pyridin-4-yl]-5-fluorophenyl}methyl carbamate

To a stirred solution of {3-[(3aR,7aR)-1-{4-[(1R,5S,6s)-6-(6-hydroxyhexyl)-3-azabicyclo[3.1.1]heptan-3-yl]pyridin-2-yl}-hexahydro-2H-pyrrolo[3,2-b]pyridin-4-yl]-5-fluorophenyl}methyl carbamate (117 mg, 0.207 mmol) in DMF (2 mL) were added IBX (173.73 mg, 0.621 mmol) at rt. The resulting mixture was stirred 12 h at rt. On completion, the mixture was extracted with EA (3×0 mL), and dried over anhydrous Na₂SO₄. After filtration, the filtrate was concentrated under reduced pressure. The residue was purified by Prep-TLC (CH₂Cl₂/MeOH 10:1) to afford the title compound (68 mg, 58% yield) as a yellow oil. LC/MS (ESI, m/z): [(M+H)]⁺=564.4.

### (1R,5S,6r)-3-(2-bromopyridin-4-yl)-6-[6-(oxan-2-yloxy)hexyl]-3-azabicyclo[3.1.1]heptane (Intermediate CW)

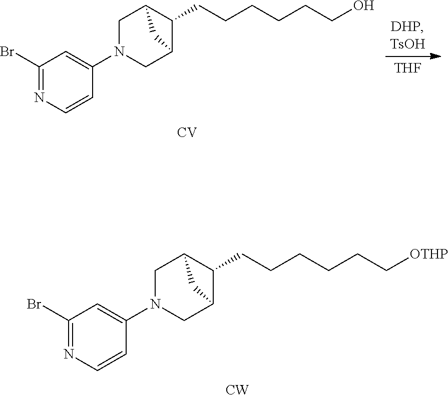

To a stirred solution of 6-[(1R,5S,6r)-3-(2-bromopyridin-4-yl)-3-azabicyclo[3.1.1]heptan-6-yl]hexan-1-ol (2.4 g, 6.8 mmol, Intermediate CV) and DHP (5.71 g, 67.9 mmol) in THF (24.00 mL) was added PPTS (341.42 mg, 1.359 mmol) at rt. The resulting mixture was stirred for 2 h at 50° C. On completion, the mixture was cooled to rt and concentrated under reduced pressure. The resulting mixture was extracted with EtOAc (3×50 mL), and dried over anhydrous Na₂SO₄. After filtration, the filtrate was concentrated under reduced pressure. The residue was purified by reverse phase flash chromatography (Column: WelFlash™ C18-I, 20-24 um, 330 g; Eluent A: Water (plus 10 mmol/L NH₄HCO₃): Eluent B: MeOH; Gradient: 25%-95% B in 30 min; Flow rate: 80 mL/min; Detector: 220/254 nm; desired fractions were collected at 92% B and concentrated under reduced pressure) to afford the title compound (2.4 g, 81% yield) as a yellow oil. LC/MS (ESI, m/z): [(M+H)]⁺=437.20, LC/MS (ESI, m/z): [(M+H+2)]⁺=439.2.

### {3-[(3aR,7aR)-1-{4-[(1R,5S,6r)-6-(6-oxohexyl)-3-azabicyclo[3.1.1]heptan-3-yl]pyridin-2-yl}-hexahydro-2H-pyrrolo[3,2-b]pyridin-4-yl]-5-fluorophenyl}methyl carbamate (Intermediate RP)

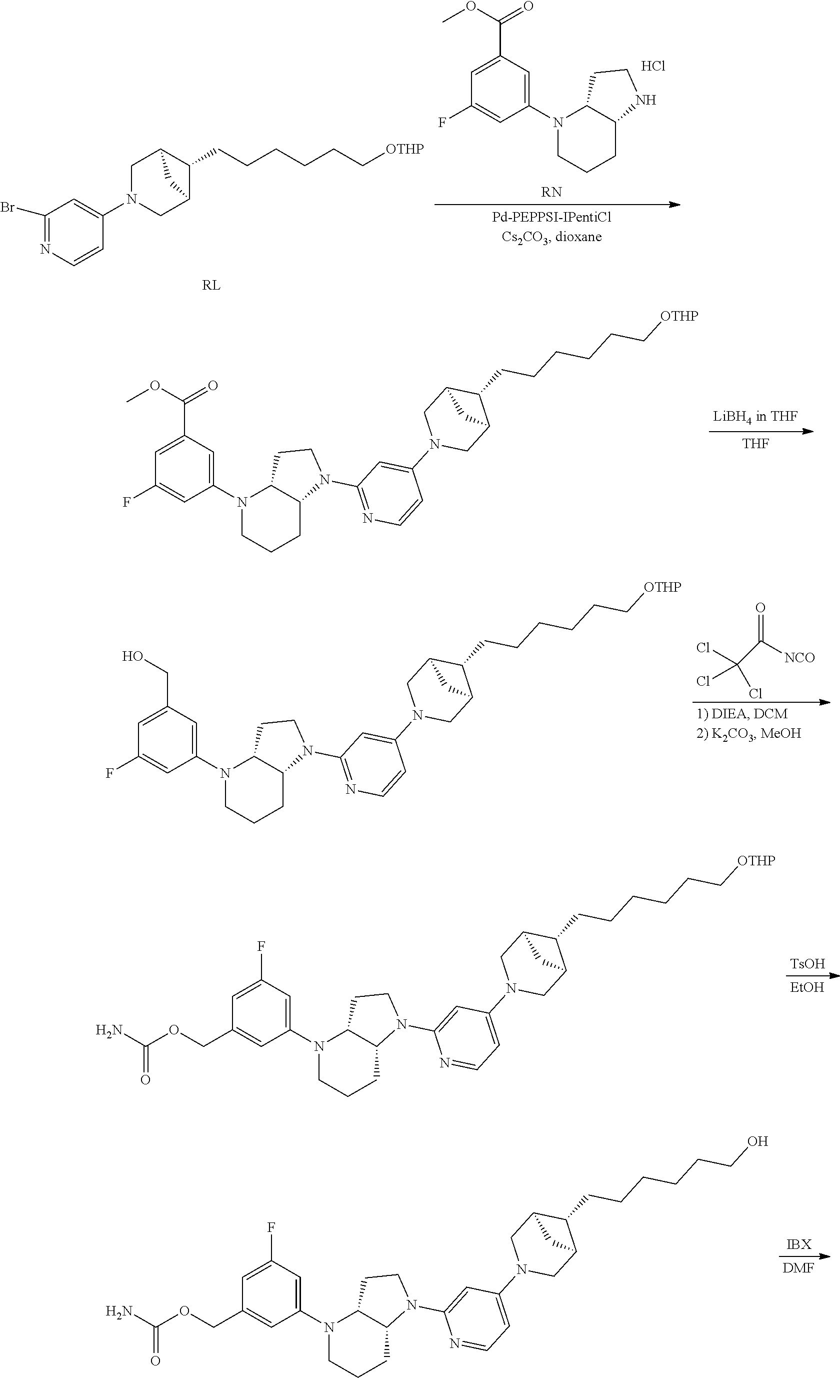

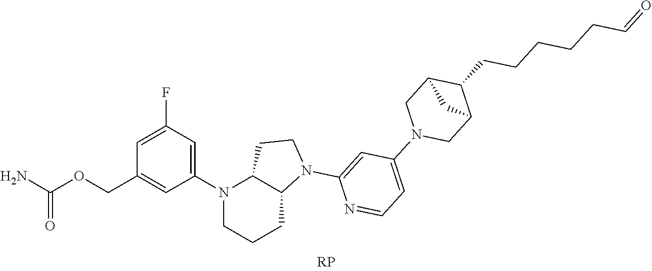 [SMILES: NC(=O)OCc1cc(F)cc(N2CCC[C@@H]3[C@H]2CCN3c2cc(N3C[C@H]4C[C@@H](C3)[C@@H]4CCCCCC=O)ccn2)c1]

### Step 1—3-[(3aR,7aR)-1-{4-[(1R,5S,6r)-6-[6-(oxan-2-yloxy)hexyl]-3-azabicyclo[3.1.1]heptan-3-yl]pyridin-2-yl}l-hexahydro-2H-pyrrolo[3,2-b]pyridin-4-yl]-5-fluorobenzoate

To a stirred solution of methyl 3-[(3aR,7aR)-octahydropyrrolo[3,2-b]pyridin-4-yl]-5-fluorobenzoate hydrochloride (720 mg, 2.29 nmol, Intermediate RN) and (1R,5S,6r)-3-(2-bromopyridin-4-yl)-6-[6-(oxan-2-yloxy)hexyl]-3-azabicyclo[3.1.1]heptane (1.00 g, 2.29 mmol, Intermediate RL) in 1,4-dioxane (10 mL) were added Pd-PEPPSI-IPentCl (288.45 mg, 0.343 mmol) and Cs₂CO₃ (1.49 g, 4.57 mmol) at rt. The resulting mixture was stirred for 2 h at 100° C. under nitrogen atmosphere. On completion, the mixture was cooled to rt and concentrated under reduced pressure. The residue product was purified by reverse phase flash (Column: WelFlash™ C18-I, 20-24 um, 330 g; Eluent A: Water (plus 10 mmol/L TFA); Eluent B: MeOH; Gradient: 25%-95% B in 30 min; Flow rate: 80 mL/min; Detector: 220/254 nm; desired fractions were collected at 62% B and concentrated under reduced pressure) to afford the title compound (350 mg, 24% yield) as a brown oil. LC/MS (ESI, m/z): [(M+H)]⁺=635.4.

### Step 2—{3-[(3aR,7aR)-1-{4-[(1R,5S,6r)-6-[6-(oxan-2-yloxy)hexyl]-3-azabicyclo[3.1.1]heptan-3-yl]pyridin-2-yl}-hexahydro-2H-pyrrolo[3,2-b]pyridin-4-yl]-5-fluorophenyl}methanol

To a stirred solution of methyl 3-[(3aR,7aR)-1-{4-[(1R,5S,6r)-6-[6-(oxan-2-yloxy)hexyl]-3-azabicyclo[3.1.1]heptan-3-yl]pyridin-2-yl}-hexahydro-2H-pyrrolo[3,2-b]pyridin-4-yl]-5-fluorobenzoate (440 mg, 0.693 mmol) in THF (10 mL) was added LiBH₄ (1.73 mL, 3.47 mmol) dropwise at 0° C. The resulting mixture was stirred for 1 h at rt under air atmosphere. On completion, the mixture was extracted with EtOAc (3×10 mL), and dried over anhydrous Na₂SO₄. After filtration, the filtrate was concentrated under reduced pressure to afford the title compound (400 mg) as a grey solid. LC/MS (ESI, m/z): [(M+H)]⁺=607.4.

### Step 3—{3-[(3aR,7aR)-1-{4-[(1R,5S,6r)-6-[6-(oxan-2-yloxy)hexyl]-3-azabicyclo[3.1.1]heptan-3-yl]pyridin-2-yl}-hexahydro-2H-pyrrolo[3,2-b]pyridin-4-yl]-5-fluorophenyl}methyl carbamate

To a stirred solution of {3-[(3aR,7aR)-1-{4-[(1R,5S,6r)-6-[6-(oxan-2-yloxy)hexyl]-3-azabicyclo[3.1.1]heptan-3-yl]pyridin-2-yl}-hexahydro-2H-pyrrolo[3,2-b]pyridin-4-yl]-5-fluorophenyl}methanol (400 mg, 0.659 mmol) and DIEA (0.23 ml, 1.31 mmol) in THF (5 mL) was added 2,2,2-trichloroacetic cyanic anhydride (496.72 mg, 2.636 mmol) at rt. The resulting mixture was stirred for 1 h at rt. Then the mixture was concentrated under reduced pressure. The residue was dissolved in MeOH (5 mL). To the above mixture was added K₂CO₃ (182.20 mg, 1.318 mmol) at rt. The reaction was stirred for 0.5 h at rt. On completion, the mixture was concentrated under reduced pressure. The resulting mixture was extracted with EtOAc (3×20 mL), and dried over anhydrous Na₂SO₄. After filtration, the filtrate was concentrated under reduced pressure to afford the title compound (740 mg) as a white solid. LC/MS (ESI, m/z): [(M+H)]⁺=650.4.

### Step 4—{3-[(3aR,7aR)-1-{4-[(1R5S,6r)-6-(6-hydroxyhexyl)-3-azabicyclo[3.1.1]heptan-3-yl]pyridin-2-yl}-hexahydro-2H-pyrrolo[3,2-b]pyridin-4-yl]-5-fluorophenyl}methyl carbamate

A solution of {3-[(3aR,7aR)-1-{4-[(1R,5S,6r)-6-[6-(oxan-2-yloxy)hexyl]-3-azabicyclo[3.1.1]heptan-3-yl]pyridin-2-yl}-hexahydro-2H-pyrrolo[3,2-b]pyridin-4-yl]-5-fluorophenyl}methyl carbamate (740 mg, 1.14 mmol) and TsOH (235.31 mg, 1.367 mmol) in EtOH (8 mL) was stirred for 1 h at 80° C. On completion, the mixture was cooled to rt and purified by reverse phase flash (Column: WelFlash™ C18-I, 20-24 um, 330 g; Eluent A: Water (plus 10 mmol/L NH4HCO₃): Eluent B: MeOH; Gradient: 40%-90% B in 30 min; Flow rate: 80 mL/min: Detector: 220/254 nm; desired fractions were collected at 86% B and concentrated under reduced pressure) to afford the title compound (200 mg, 31% yield) as a white solid. LC/MS (ESI, m/z): [(M+H)]⁺=566.4.

### Step 5—{3-[(3aR,7aR)-1-{4-[(1R,5S,6r)-6-(6-oxohexyl)-3-azabicyclo[3.1.1]heptan-3-yl]pyridin-2-yl}-hexahydro-2H-pyrrolo[3,2-b]pyridin-4-yl]-5-fluorophenyl}methyl carbamate

To a stirred solution of {3-[(3aR,7aR)-1-{4-[(1R,5S,6r)-6-(6-hydroxyhexyl)-3-azabicyclo[3.1.1]heptan-3-yl]pyridin-2-yl}-hexahydro-2H-pyrrolo[3,2-b]pyridin-4-yl]-5-fluorophenyl}methyl carbamate (160 mg, 0.283 mmol) in DMF (2 mL) was added IBX (237.58 mg, 0.849 mmol) at rt. The resulting mixture was stirred for 16 h at rt. On completion, the mixture was purified by Prep-TLC (CH₂Cl₂/MeOH 10:1) to afford the title compound (60 mg, 38% yield) as a light yellow solid. LC/MS (ESI, m/z): [(M+H)]⁺=564.3.

### 5-[4-(8-Bromooctyl)benzoyl]-N-[(1S)-1-(3-chlorophenyl)-2-cyanoethyl]-4-(3,6-difluoro-2-methylphenyl)-1-methylpyrrole-3-carboxamide (Intermediate RQ)

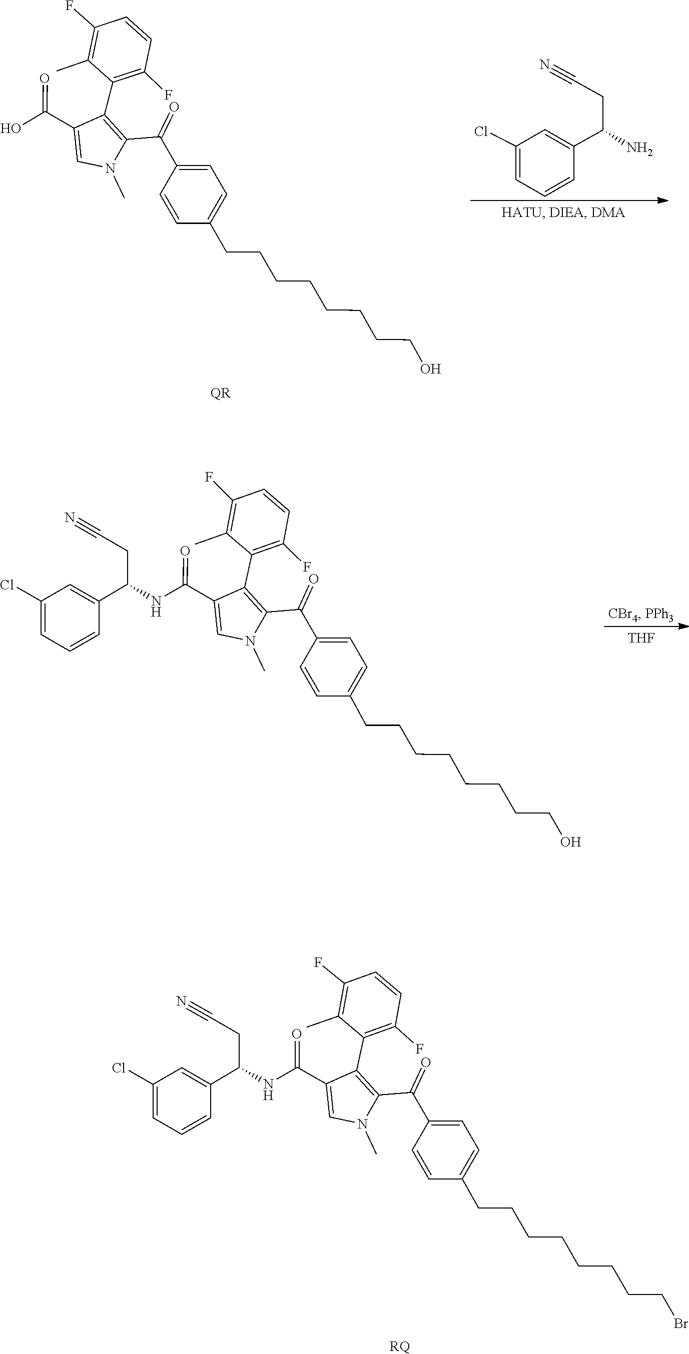

### Step 1—N-[(1S)-1-(3-chlorophenyl)-2-cyanoethyl]-4-(3,6-difluoro-2-methylphenyl)-5-[4-(8-hydroxyoctyl)benzoyl]-1-methylpyrrole-3-carboxamide

To a stirred solution of 4-(3,6-difluoro-2-methylphenyl)-5-[4-(8-hydroxyoctyl)benzoyl]-1-methylpyrrole-3-carboxylic acid (200 mg, 0.414 mmol, Intermediate QR) and DIEA (110 mg, 0.851 mmol) in DMA (5 mL) were added (3S)-3-amino-3-(3-chlorophenyl)propanenitrile hydrochloride (140 mg, 0.645 mmol, CAS #1228558-95-9) and HATU (240 mg, 0.631 mmol) in portions at rt under air atmosphere. The resulting mixture was stirred for 1 h at rt under air atmosphere. On completion, the mixture was purified by reverse-phase flash chromatography (column, C18 silica gel: mobile phase, MeCN in Water (10 mmol/L NH4HCO3), 45% to 70% gradient in 25 min: detector, UV 254 nm) to give the title compound (250 mg, 94% yield) as a white solid. LC/MS (ESI, m/z): [(M+H)]⁺=646.3.

### Step 2—5-[4-(8-Bromooctyl)benzoyl]-N-[(1S)-1-(3-chlorophenyl)-2-cyanoethyl]-4-(3,6-difluoro-2-methylphenyl)-1-methylpyrrole-3-carboxamide

To a stirred mixture of N-[(1S)-1-(3-chlorophenyl)-2-cyanoethyl]-4-(3,6-difluoro-2-methylphenyl)-5-[4-(8-hydroxyoctyl)benzoyl]-1-methylpyrrole-3-carboxamide (100 mg, 0.155 mmol) in THF (5 mL) were added CBr₄ (102.64 mg, 0.310 mmol) and PPh₃ (81.18 mg, 0.310 mmol) in portions at rt under air atmosphere. The resulting mixture was stirred for 2 h at rt under air atmosphere. On completion, the mixture was purified by prep-TLC (PE/EA 1:1) to afford the title compound (100 mg, 91% yield) as a white solid. LC/MS (ESI, m/z): [(M−H)]⁻=706.2.

### 4-((2-(2-(Benzyloxy)ethoxy)ethoxy)methyl)piperidine (Intermediate RR)

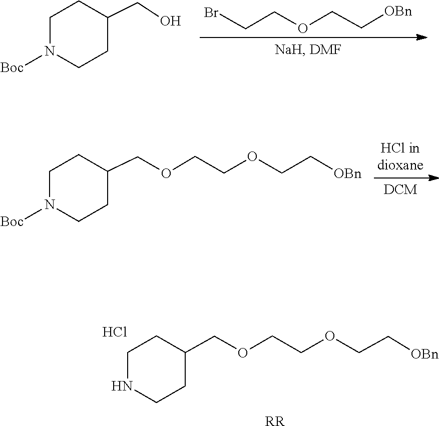 [SMILES: CC(C)(C)OC(=O)N1CCC(CO)CC1 ; BrCCOCCOCc1ccccc1 ; CC(C)(C)OC(=O)N1CCC(COCCOCCOCc2ccccc2)CC1 ; Cl ; c1ccc(COCCOCCOCC2CCNCC2)cc1 ; "RR"]

### Step 1—Tert-butyl 4-((2-(2-(benzyloxy)ethoxy)ethoxy)methyl)piperidine-1-carboxylate

To a stirred mixture of tert-butyl 4-(hydroxymethyl) piperidine-1-carboxylate (5 g, 20 mmol, CAS #123855-51-6) and NaH (1.40 g, 35.1 mmol) in DMF (50 mL) was added {[2-(2-bromoethoxy) ethoxy]methyl}benzene (6.62 g, 25.6 mmol, CAS #125562-32-5) in portions at 0 n° C. under nitrogen atmosphere. The resulting mixture was stirred for 16 h at rt under nitrogen atmosphere. On completion, the reaction was quenched with sat. NH₄Cl (aq.) at 0° C. The aqueous layer was extracted with EtOAc (3×500 mL). The resulting mixture was concentrated under reduced pressure. The mixture was purified by reverse phase flash chromatography (Column: WelFlash™ C18-I, 20-40 μm, 120 g; Eluent A: Water (plus 10 mmol/L NH₄HCO₃); Eluent B: ACN; Gradient: 75%-95% B in 25 min; Flow rate: 60 mL/min; Detector: 220/254 nm; desired fractions were collected at 80% B and concentrated under reduced pressure) to give the title compound (2.5 g, 27% yield) as a yellow liquid. ¹H NMR (400 MHz, Chloroform-d) δ 7.41-7.23 (m, 5H), 4.59 (s, 2H), 4.10 (d, J=12.5 Hz, 2H), 3.73-3.66 (m, 2H), 3.70-3.59 (m, 4H), 3.63-3.57 (m, 2H), 3.32 (d, J=6.2 Hz, 2H), 2.70 (t, J=12.9 Hz, 2H), 1.80-1.67 (m, 3H), 1.47 (s, 9H), 1.20-1.06 (m, 2H). LC/MS (ESI, m/z): [(M+H)]⁺=394.5.

### Step 2—4-((2-(2-(Benzyloxy)ethoxy)ethoxy)methyl)piperidine

To a stirred mixture of tert-butyl 4-({2-[2-(benzyloxy) ethoxy]ethoxy}methyl) piperidine-1-carboxylate (2.5 g, 6.4 mmol) in DCM (15 mL) was added HCl (gas) in 1,4-dioxane (15 mL, 60 mmol) dropwise at rt. The resulting mixture was stirred for 1 h at rt. On completion, the mixture was concentrated under reduced pressure to give the title compound (2.5 g) as a yellow oil. LC/MS (ESI, m/z): [(M+H)]⁺=294.2.

### (3aR,7aR)-4-(3-fluorophenyl)-1-(4-(4-((2-(2-iodoethoxy)ethoxy)methyl)piperidin-1-yl)pyridin-2-yl)octahydro-1H-pyrrolo[3,2-b]pyridine (Intermediate RS)

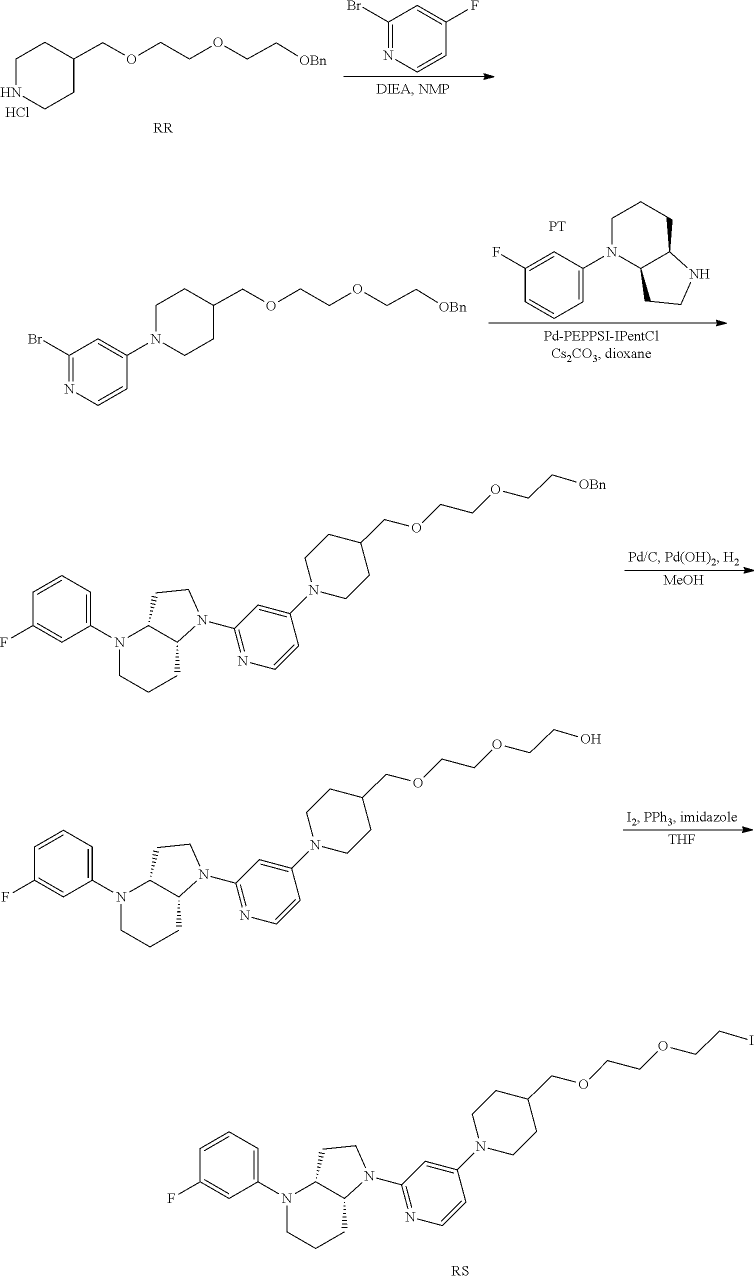

### Step 1—4-(4-((2-(2-(benzyloxy)ethoxy)ethoxy)methyl)piperidin-1-yl)-2-bromopyridine

To a stirred mixture of 4-({2-[2-(benzyloxy) ethoxy]ethoxy}methyl) piperidine (2.5 g, 8.5 mmol, Intermediate RR) and 2-bromo-4-fluoropyridine (1.80 g, 10.2 mmol, CAS #357927-50-5) in NMP (25 mL) was added DIEA (3.30 g, 25.6 mmol) dropwise at rt. The resulting mixture was stirred for 1 h at 130° C. On completion, the mixture was cooled to rt and purified by reverse phase flash chromatography (Column: WelFlash™ C18-I, 20-40 μm, 120 g; Eluent A: Water (plus 10 mmol/L NH₄HCO₃): Eluent B: ACN; Gradient: 65%-85% B in 25 min; Flow rate: 60 mL/min; Detector: 220/254 nm; desired fractions were collected at 70% B and concentrated under reduced pressure) to give the title compound (2.6 g, 68% yield) as a yellow liquid. ¹H NMR (300 MHz, Chloroform-d) δ 7.97 (d, J=6.0 Hz, 1H), 7.40-7.26 (m, 5H), 6.80 (d, J=2.3 Hz, 1H), 6.59 (dd, J=6.1, 2.4 Hz, 1H), 4.60 (s, 2H), 3.83 (dt, J=13.2, 3.2 Hz, 2H), 3.76-3.57 (m, 8H), 3.36 (d, J=6.1 Hz, 2H), 2.88 (td, J=12.8, 2.6 Hz, 2H), 1.87 (m, J=14.7, 9.3, 4.9 Hz, 3H), 1.38-1.19 (in, 2H); LC/MS (ESI, m/z): [(M+H)]⁺=449.3.

### Step 2—(3aR,7aR)-1-(4-(4-((2-(2-(benzyloxy)ethoxy)ethoxy)methyl)piperidin-1-yl)pyridin-2-yl)-4-(3-fluorophenyl)octahydro-1H-pyrrolo[3,2-b]pyridine

To a stirred mixture of 4-[4-({2-[2-(benzyloxy)ethoxy]ethoxy}methyl)piperidin-1-yl]-2-bromopyridine (1 g, 2 mmol) and (3aR,7aR)-4-(3-fluorophenyl)-octahydropyrrolo[3,2-b]pyridine (0.58 g, 2.63 mmol, Intermediate PT) in dioxane (10 mL) were added Cs₂CO₃ (2.18 g, 6.70 mmol) and {1,3-bis[2,6-bis(pentan-3-yl)phenyl]-4,5-dichloro-2,3-dihydro-1H-imidazol-2-yl}dichloro(2-methyl-llambda4-pyridin-1-yl)palladium (0.19 g, 0.22 mmol) in portions at rt. The resulting mixture was stirred for 16 h at 100° C. under nitrogen atmosphere. On completion, the mixture was cooled to rt and purified by reverse phase flash chromatography (Column: WelFlash™ C18-I, 20-40 μm, 120 g; Eluent A: Water (plus 10 mmol/L TFA); Eluent B: ACN: Gradient: 30%-50% B in 25 min; Flow rate: 60 mL/min; Detector: 220/254 nm; desired fractions were collected at 42% B and concentrated under reduced pressure) to give the title compound (960 mg, 73% yield) as a brown solid. ¹H NMR (400 MHz, Chloroform-d) δ 7.75 (s, 1H), 7.32 (dd, J=8.6, 4.6 Hz, 1H), 7.22 (q, J=7.9 Hz, 1H), 6.69 (d, J=8.3 Hz, 1H), 6.61 (d, J=12.7 Hz, 1H), 6.54 (t, J=8.1 Hz, 1H), 6.27 (d, J=6.7 Hz, 1H), 5.53 (s, 1H), 5.32 (s, 1H), 4.59 (s, 2H), 4.50 (s, 1H), 4.24 (s, 1H), 3.93 (d, J=13.3 Hz, 2H), 3.67 (m, J=19.9, 9.4, 6.5, 3.9 Hz. 9H), 3.49 (d, J=12.1 Hz, 3H), 3.38 (d, J=6.1 Hz, 2H), 3.05 (t, J=12.5 Hz, 2H), 2.90 (t, J=11.8 Hz, 1H), 2.34 (d, J=12.7 Hz, 1H), 2.19 (s, 1H), 2.10 (d, J=10.4 Hz, OH), 1.99 (s, 2H), 1.91 (t, J=13.6 Hz, 3H), 1.75-1.64 (m, 1H), 1.39-1.25 (m, 5H); LC/MS (ESI, m/z): [(M+H)]⁺=589.8.

### Step 3—(3aR,7aR)-1-(4-(4-((2-(2-(benzyloxy)ethoxy)ethoxy)methyl)piperidin-1-yl)pyridin-2-yl)-4-(3-fluorophenyl)octahydro-1H-pyrrolo[3,2-b]pyridine

To a stirred mixture of 2-[(3aR,7aR)-4-(3-fluorophenyl)-hexahydro-2H-pyrrolo[3,2-b]pyridin-1-yl]-4-[4-({2-[2-(benzyloxy) ethoxy]ethoxy}methyl) piperidin-1-yl]pyridine (960 mg, 1.63 mmol) in MeOH (10 mL) were added Pd/C (250 mg, 2.35 mmol) and Pd(OH)₂/C (250 mg, 1.78 mmol) in portions at rt. The resulting mixture was stirred for 1 h at rt under hydrogen atmosphere. On completion, the mixture was filtered, and the filter cake was washed with MeOH (5×30 mL). The filtrate was concentrated under reduced pressure to give the title compound (590 mg. 73% yield) as a brown oil. ¹H NMR (400 MHz, Chloroform-d) δ 7.89 (s, 1H), 7.21 (q, J=8.0 Hz, 1H), 6.68 (d, J=8.4 Hz, 1H), 6.64-6.49 (m, 1H), 6.28 (s, OH), 5.55 (s, 1H), 4.77 (s, 1H), 4.48 (dd, J=12.2, 6.3 Hz, 1H), 4.31 (s, 1H), 3.96 (d, J=12.9 Hz, 2H), 3.76 (s, 2H), 3.70 (s, 2H), 3.64 (s, 4H), 3.52 (s, 4H), 3.46 (d, J=15.1 Hz, 1H), 3.46 (s, 1H), 3.39 (d, J=6.1 Hz, 2H), 3.07 (t, J=12.6 Hz, 1H), 2.89 (t, J=11.5 Hz, 1H), 2.55-2.05 (m, 8H), 2.05-1.61 (m, 3H); LC/MS (ESI, m/z): [(M+H)]=498.3.

### Step 4—(3aR,7aR)-4-(3-fluorophenyl)-1-(4-(4-((2-(2-iodoethoxy)ethoxy)methyl)piperidin-1-yl)pyridin-2-yl)octahydro-1H-pyrrolo[3,2-b]pyridine

To a stirred mixture of 2-{2-[(1-{2-[(3aR,7aR)-4-(3-fluorophenyl)-hexahydro-2H-pyrrolo[3,2-b]pyridin-1-yl]pyridin-4-yl}piperidin-4-yl)methoxy]ethoxy}ethanol (200 mg, 0.401 mmol) and PPh₃ (263 mg, 1.00 mmol) in THF (2 mL) were added 1H-imidazole (68 mg, 1.0 mmol) and I2 (407 mg, 1.61 mmol) in portions at rt. The resulting mixture was stirred for 2 h at rt. On completion, the mixture was purified by Prep-TLC (DCM/MeOH 15:1) to afford the title compound (200 mg, 81.94%) as a reddish brown semi-solid. ¹H NMR (400 MHz, Chloroform-d) δ 7.75-7.63 (m, 2H), 7.51 (td, J=7.0, 6.2, 4.1 Hz, 1H), 7.23 (td, J=8.2, 7.0 Hz, 1H), 6.74 (dd, J=8.5, 2.4 Hz, 1H), 6.62 (dt, J=12.6, 2.4 Hz, 1H), 6.40 (dd, J=7.8, 2.4 Hz, 1H), 4.60 (dt, J=12.6, 6.6 Hz, 1H), 4.28 (s, 1H), 4.01 (d, J=13.4 Hz, 2H), 3.79 (t, J=6.8 Hz, 2H), 3.77-3.67 (m, 1H), 3.71-3.56 (m, 4H), 3.49 (d, J=12.5 Hz, 1H), 3.29 (t, J=6.8 Hz, 2H), 3.16 (t, J=12.7 Hz, 2H), 2.99-2.88 (m, 1H), 2.41 (d, J=10.8 Hz, 1H), 2.30-2.10 (m, 2H), 2.06 (d, J=6.1 Hz, OH), 2.03 (s, 2H), 1.98 (d, J=15.4 Hz, 3H), 1.91 (s, OH), 1.79 (q, J=12.9 Hz, 1H), 1.42 (d, J=12.9 Hz, 4H), 1.28 (s, 1H); LC/MS (ESI, m/z): [(M+H)]⁺=608.5.

### 4-(3,6-Difluoro-2-methylphenyl)-5-(4-{12-[2-(2-hydroxyethoxy)ethoxy]ethyl}benzoyl)-1H-pyrrole-3-carboxylic acid (Intermediate RT)

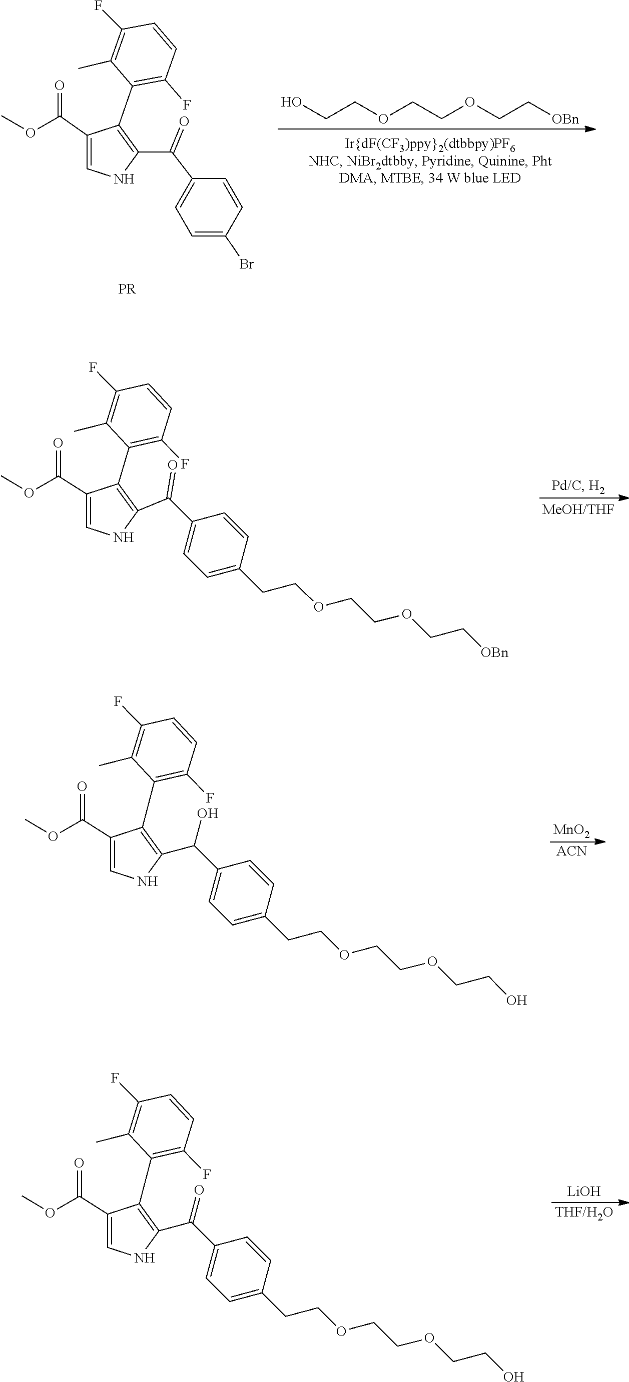

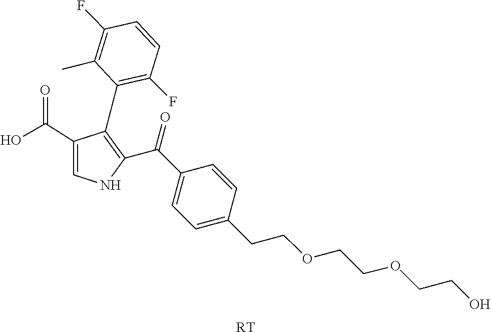 [SMILES: Cc1c(F)ccc(F)c1-c1c(C(=O)O)c[nH]c1C(=O)c1ccc(CCOCCOCCO)cc1 ; "RT"]

### Step 1—5-[4-(2-{2-[2-(Benzyloxy)ethoxy]ethoxy}ethyl)benzoyl]-4-(3,6-difluoro-2-methylphenyl)-1H-pyrrole-3-carboxylate

To a stirred mixture of 2-{2-[2-(benzyloxy)ethoxy]ethoxy}ethanol (1.25 g, 5.18 mmol, CAS #55489-58-2) in 2-methoxy-2-methylpropane (20 mL) was added 5,7-di-tert-butyl-3-phenyl-1,3lambda5-benzoxazol-3-ylium boron trifluoride fluoride (2.05 g, 5.18 mmol) at rt under nitrogen atmosphere. The mixture was stirred for 20 min at rt under nitrogen atmosphere. To the above mixture was added pyridine (0.27 g, 3.45 mmol) at rt. The resulting mixture was stirred for additional 30 min at room temperature. The resulting mixture was filtered, the filter cake was washed with 2-methoxy-2-methylpropane (20 mL). The filtrate was then used directly. To a stirred mixture of methyl 5-(4-bromobenzoyl)-4-(3,6-difluoro-2-methylphenyl)-1H-pyrrole-3-carboxylate (1.5 g, 3.5 mmol, Intermediate PR) and nickel catalyst (84.10 mg, 0.173 mmol) in DMA (20 mL) were added phthalimide (101.65 mg, 0.691 mmol) and 1-azabicyclo[2.2.2]octane (672.12 mg, 6.045 mmol) at rt under nitrogen atmosphere. To the above mixture was added filtrate at rt. The vial was stirred at 1500 rpm stir rate and irradiated under 450 nm LED modules at 100% light intensity in a PennOC Integrated Photoreactor for 2 h. On completion, the mixture was concentrated under reduced pressure. The residue was purified by silica gel column chromatography, eluted with PE/EA (1:1), to afford the title compound (1 g, 50% yield) as a light yellow oil. LC/MS (ESI, m/z): [(M+H)]⁺=578.3.

### Step 2—Methyl 4-(3,6-difluoro-2-methylphenyl)-5-[hydroxy(4-{2-[2-(2-hydroxyethoxy)ethoxy]ethyl}phenyl)methyl]-1H-pyrrole-3-carboxylate

A mixture of methyl 5-[4-(2-{2-[2-(benzyloxy)ethoxy]ethoxy}ethyl)benzoyl]-4-(3,6-difluoro-2-methylphenyl)-1H-pyrrole-3-carboxylate (800 mg, 1.39 mmol) and Pd/C (200 mg, 1.88 mmol) in MeOH (20 mL) was stirred for 2 h at rt under hydrogen atmosphere. On completion, the mixture was filtered, the filter cake was washed with MeOH (3×30 mL). The filtrate was concentrated under reduced pressure to give the title compound (500 mg, 74% yield) as a brown yellow oil. LC/MS (ESI, m/z): [(M+H)]⁺=490.2.

### Step 3—Methyl 4-(3,6-difluoro-2-methylphenyl)-5-(4-{2-[2-(2-hydroxyethoxy)ethoxy]ethyl}benzoyl)-1H-pyrrole-3-carboxylate

A mixture of methyl 4-(3,6-difluoro-2-methylphenyl)-5-[hydroxy(4-{2-[2-(2-hydroxyethoxy)ethoxy]ethyl}phenyl)methyl]-1H-pyrrole-3-carboxylate (500 mg, 1.02 mmol) and MnO₂ (443.99 mg, 5.105 mmol) in ACN (8 mL) was stirred for 2 h at 60° C. under air atmosphere. On completion, the mixture was filtered, the filter cake was washed with MeCN (3×10 mL). The filtrate was concentrated under reduced pressure to give the title compound (150 mg, 30% yield) as a yellow oil. LC/MS (ESI, m/z): [(M+H)]⁺=488.2.

### Step 4—4-(3,6-Difluoro-2-methylphenyl)-5-(4-{2-[2-(2-hydroxyethoxy)ethoxy]ethyl}benzoyl)-1H-pyrrole-3-carboxylic acid

A mixture of methyl 4-(3,6-difluoro-2-methylphenyl)-5-(4-{2-[2-(2-hydroxyethoxy)ethoxy]ethyl}benzoyl)-1H-pyrrole-3-carboxylate (300 mg, 0.615 mmol) and LiOH·H₂O (15.00 mL, 29.98 mmol) in THF (15 mL) was stirred for 16 h at 60° C. under atmosphere. On completion, the mixture was cooled to rt and acidified to pH 4 with cone. HCl. The aqueous layer was extracted with EtOAc (3×30 mL). The organic layer was concentrated under reduced pressure. The residue was purified by reverse-phase flash chromatography (column, C18 silica gel; mobile phase, MeCN in Water (10 mmol/L NH₄HCO₃), 20% to 50% gradient in 25 min; detector, UV 254 nm) to afford the title compound (220 mg, 76% yield) as a white solid. ¹H NMR (400 MHz, DMSO-d6) δ 12.65 (d, J=3.4 Hz, 1H), 11.99 (s, 1H), 7.76 (d, J=3.4 Hz, 1H), 7.33-7.24 (m, 2H), 7.08 (d, J=7.9 Hz, 2H), 6.90 (dd. J=9.0, 4.4 Hz, 1H), 6.75 (dd, J=8.9, 4.5 Hz, 1H), 4.57-4.32 (m, 1H), 3.57-3.46 (m, 8H), 3.42 (dd, J=6.0, 4.9 Hz, 2H), 2.75 (d, J=7.0 Hz, 2H), 1.85 (d, J=2.4 Hz, 3H); LC/MS (ESI, m/z): [(M+H)]⁺=474.2.

### (3S)-3-{[5-(4-{2-[2-(2-bromoethoxy)ethoxy]ethyl}benzoyl)-4-(3,6-difluoro-2-methylphenyl)-1H-pyrrol-3-yl]formamido}-3-(3-chlorophenyl)propenamide (Intermediate RU)

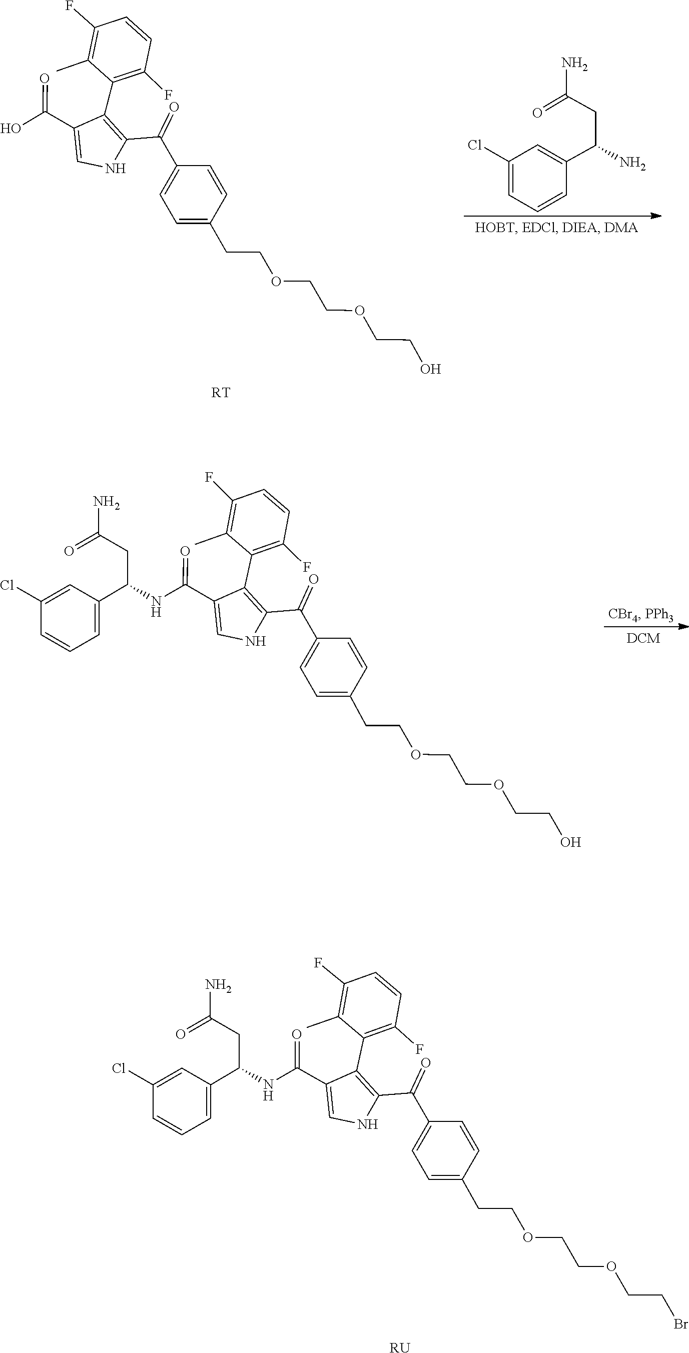

### Step 1—(3S)-3-(3-chlorophenyl)-3-{[4-(3,6-difluoro-2-methylphenyl)-5-(4-{2-[2-(2-hydroxyethoxy)ethoxy]ethyl}benzoyl)-1H-pyrrol-3-yl]formamido}propenamide

To a stirred mixture of 4-(3,6-difluoro-2-methylphenyl)-5-(4-{2-[2-(2-hydroxyethoxy)ethoxy]ethyl}benzoyl)-1H-pyrrole-3-carboxylic acid (200 mg, 0.422 mmol, Intermediate RT) and EDCI (121.46 mg, 0.633 mmol), DIEA (163.79 mg, 1.266 mmol) in DMA (4 mL) was added (3S)-3-amino-3-(3-chlorophenyl)propanamide (167.82 mg, 0.844 mmol) at rt under air atmosphere. The resulting mixture was stirred for 2 h at rt under air atmosphere. On completion, the mixture was purified by reverse-phase flash chromatography (column, C18 silica gel; mobile phase, MeCN in Water (10 mmol/L NH₄HCO₃), 25% to 50% gradient in 20 min; detector, UV 254 nm) to afford the title compound (200 mg, 72% yield) as a white solid. ¹H NMR (400 MHz, Chloroform-d) δ 11.15 (d, J=20.5 Hz, 1H), 7.78 (d, J=5.8 Hz, 1H), 7.36 (d, J=7.9 Hz, 2H), 7.25-7.16 (m, 3H), 7.13-7.01 (m, 4H), 6.97 (dd, J=7.4, 4.2 Hz, 1H), 6.77-6.56 (m, 2H), 6.07-5.80 (m, 2H), 5.45-5.32 (m, 1H), 3.80-3.42 (m, 10H), 2.84 (d, J=6.6 Hz, 2H), 2.71-2.39 (m, 3H), 1.91-1.81 (m, 2H); LC/MS (ESI, m/z): [(M+H)]=654.1.

### Step 2—(3S)-3-{[5-(4-{2-[2-(2-bromoethoxy)ethoxy]ethyl}benzoyl)-4-(3,6-difluoro-2-methylphenyl)-1H-pyrrol-3-yl]formamido}-3-(3-chlorophenyl)propanamide

To a stirred mixture of (3S)-3-(3-chlorophenyl)-3-{[4-(3,6-difluoro-2-methylphenyl)-5-(4-{2-[2-(2-hydroxyethoxy)ethoxy]ethyl}benzoyl)-1H-pyrrol-3-yl]formamido}propanamide (100 mg, 0.153 mmol) and PPh₃ (60.15 mg, 0.229 mmol) in DCM (2 mL) was added CBr₄ (60.84 mg, 0.184 mmol) at rt under air atmosphere. The resulting mixture was stirred for 2 h at rt under air atmosphere. On completion, the mixture was concentrated under reduced pressure. The residue was purified by reverse-phase flash chromatography (column, C18 silica gel; mobile phase, MeCN in Water (10 mmol/L NH4HCO₃), 30% to 65% gradient in 25 min; detector, UV 254 nm) to afford the title compound (100 mg, 91% yield) as a light yellow solid. ¹H NMR (400 MHz, Chloroform-d) δ 10.79 (d, J=28.2 Hz, 1H), 7.88 (d, J=16.0 Hz, 1H), 7.31 (s, 1H), 7.22 (d, J=3.7 Hz, 2H), 7.15-7.04 (m, 1H), 6.98 (d, J=7.9 Hz, 2H), 6.83-6.53 (m, 2H), 5.42 (s, 1H), 3.82 (d, J=6.3 Hz, 2H), 3.68 (dd, J=5.9, 3.2 Hz, 2H), 3.64-3.56 (m, 3H), 3.48-3.21 (m, 2H), 2.81 (d, J=7.0 Hz, 2H), 2.68 (d, J=5.5 Hz, 2H), 2.10-1.97 (m, 2H), 1.86 (dd, J=23.2, 2.4 Hz, 3H): LC/MS (ESI, m/z): [(M+H)]+=717.0.

### 5-{4-[3-(2-Bromoethoxy)propyl]benzoyl}-4-(3,6-difluoro-2-methylphenyl)-1H-pyrrole-3-carboxylic acid (Intermediate RV)

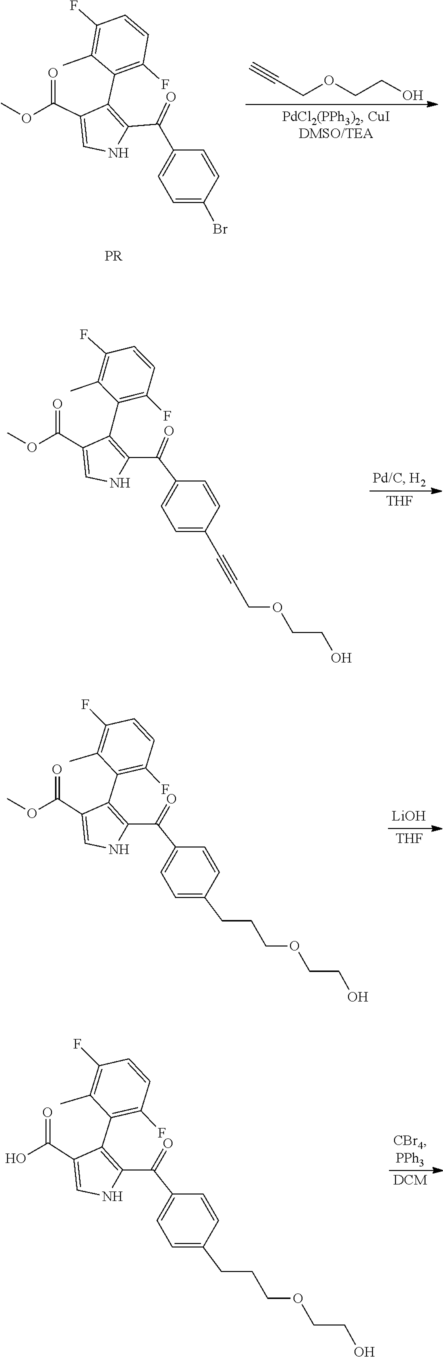

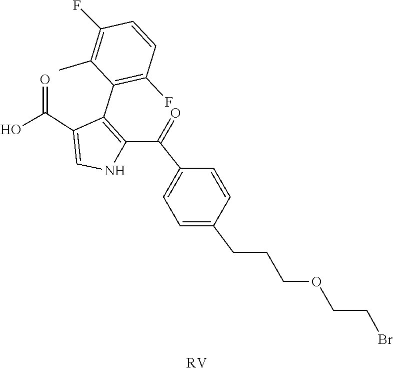 [SMILES: Cc1c(F)ccc(F)c1-c1c(C(=O)O)c[nH]c1C(=O)c1ccc(CCCOCCBr)cc1 ; "RV"]

### Step 1—4-(3,6-Difluoro-2-methylphenyl)-5-{4-[3-(2-hydroxyethoxy)prop-1-yn-1-yl]benzoyl}-1H-pyrrole-3-carboxylate

To a stirred solution of methyl 5-(4-bromobenzoyl)-4-(3,6-difluoro-2-methylphenyl)-1H-pyrrole-3-carboxylate (880 mg, 2.03 mmol, Intermediate PR) and 2-(prop-2-yn-1-yloxy)ethanol (305 mg, 3.046 mmol, CAS #3973-18-0) in DMSO (15 mL) and TEA (5 mL) were added Tetrakis(triphenylphosphine)palladium(0) (234 mg, 0.202 mmol) and CuI (77 mg, 0.404 mmol) in portions at rt under nitrogen atmosphere. The resulting mixture was stirred for 2 h at 80° C. under air atmosphere. On completion, the mixture was cooled to rt and concentrated under reduced pressure. The residue was purified by silica gel column chromatography, eluted with PE/EA (1:1), to afford the title compound (900 mg, 98% yield) as a yellow oil. LC/MS (ESI, m/z): [(M+H)]⁺=453.4.

### Step 2—Methyl 4-(3,6-difluoro-2-methylphenyl)-5-{4-[3-(2-hydroxyethoxy)propyl]benzoyl}-1H-pyrrole-3-carboxylate

A solution of methyl 4-(3,6-difluoro-2-methylphenyl)-5-{4-[3-(2-hydroxyethoxy)prop-1-yn-1-yl]benzoyl}-1H-pyrrole-3-carboxylate (750 mg, 1.65 mmol) and Pd/C (400 mg, 3.76 mmol) in THF (30 mL) was stirred for 3 h at rt under hydrogen atmosphere. On completion, the mixture was filtered, and the filter cake was washed with ethanol (3×1 mL). The filtrate was concentrated under reduced pressure to give the title compound (850 mg, 90% yield) as a yellow oil. LC/MS (ESI, m/z): [(M+H)]⁺=457.5.

### Step 3—4-(3,6-Difluoro-2-methylphenyl)-5-{14-[3-(2-hydroxyethoxy)propyl]benzoyl}-1H-pyrrole-3-carboxylic acid

To a stirred mixture of methyl 4-(3,6-difluoro-2-methylphenyl)-5-{4-[3-(2-hydroxyethoxy)propyl]benzoyl}-1H-pyrrole-3-carboxylate (800 mg, 1.75 mmol) in THF (30 mL) was added LiOH·H₂O (30 mL, 60 mmol) dropwise at rt under air atmosphere. The resulting mixture was stirred for 16 h at 80° C. under air atmosphere. On completion, the mixture was cooled to rt and concentrated under reduced pressure. The residue was dissolved in water (20 mL). The mixture was acidified to pH 6 with cone. HCl. The resulting mixture was concentrated under reduced pressure. The mixture was purified by reverse phase flash chromatography (Column: WelFlash™ C18-I, 20-40 um, 120 g; Eluent A: Water (plus 10 mmol/L NH4HCO₃); Eluent B: ACN; Gradient: 30%-50% B in 25 min; Flow rate: 60 mL/min: Detector: 220/254 nm; desired fractions were collected at 42% B and concentrated under reduced pressure) to afford the title compound (530 mg, 68% yield) as a white solid. LC/MS (ESI, m/z): [(M+H)]⁺=443.4.

### Step 4—5-{4-[3-(2-Bromoethoxy)propyl]benzoyl}-4-(3,6-difluoro-2-methylphenyl)-1H-pyrrole-3-carboxylic acid

A solution of 4-(3,6-difluoro-2-methylphenyl)-5-{4-[3-(2-hydroxyethoxy)propyl]benzoyl}-1H-pyrrole-3-carboxylic acid (500 mg, 1.13 mmol) and PPh₃ (590 mg, 2.25 mmol) in DCM (30 mL) was stirred for 2 h at rt under air atmosphere. On completion, the mixture was purified by silica gel column chromatography, eluted with PE/EA (1:1), to afford the title compound (170 mg, 30% yield) as a yellow solid. LC/MS (ESI, m/z): [(M+H)]⁺=506.3.

### (3S)-3-[(5-{4-[3-(2-bromoethoxy)propyl]benzoyl}-4-(3,6-difluoro-2-methylphenyl)-1H-pyrrol-3-yl)formamido]-3-(3-chlorophenyl)propanamide (Intermediate RW)

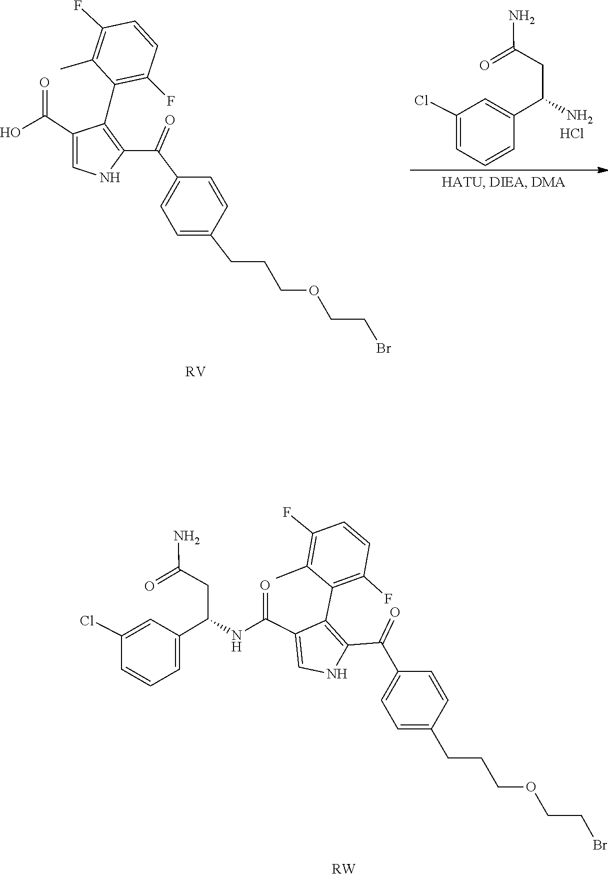 [SMILES: Cc1c(F)ccc(F)c1-c1c(C(=O)O)c[nH]c1C(=O)c1ccc(CCCOCCBr)cc1 ; NC(=O)C[C@H](N)c1cccc(Cl)c1 ; Cl ; "RV" ; Cc1c(F)ccc(F)c1-c1c(C(=O)N[C@@H](CC(N)=O)c2cccc(Cl)c2)c[nH]c1C(=O)c1ccc(CCCOCCBr)cc1 ; "RW"]

To a stirred solution of 5-{4-[3-(2-bromoethoxy)propyl]benzoyl}-4-(3,6-difluoro-2-methylphenyl)-1H-pyrrole-3-carboxylic acid (90 mg, 0.18 mmol, Intermediate RV) and (3S)-3-amino-3-(3-chlorophenyl)propanamide (43 mg, 0.22 mmol) in DMA (3 mL) were added DIEA (70 mg, 0.54 mmol) and HATU (102 mg, 0.268 mmol) in portions at rt under air atmosphere. The resulting mixture was stirred for 2 h at rt under air atmosphere. On completion, the mixture was concentrated under reduced pressure. The residue was purified by reverse phase flash chromatography (Column: WelFlash™ C18-I, 20-40 um, 120 g; Eluent A: Water (plus 10 mmol/L NH₄HCO₃); Eluent B: ACN; Gradient: 45%-65% B in 25 min: Flow rate: 60 mL/min; Detector: 220/254 nm; desired fractions were collected at 48% B and concentrated under reduced pressure) to afford the title compound (70 mg, 57% yield) as a white solid. LC/MS (ESI, m/z): [(M+H)]⁺=687.0.

### 2-(1-{12-[(3aR,7aR)-4-(3-fluorophenyl)-hexahydro-2H-pyrrolo[3,2-b]pyridin-1-yl]pyridin-4-yl}piperidin-4-yl)acetaldehyde (Intermediate RX)

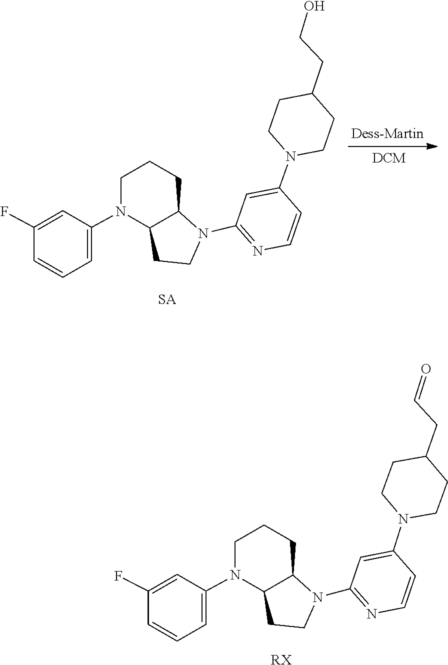

### Step 1—2-(1-(2-((3aR,7aR)-4-(3-fluorophenyl)octahydro-1H-pyrrolo[3,2-b]pyridin-1-yl)pyridin-4-yl)piperidin-4-yl)acetaldehyde

To a stirred solution of 2-(1-{2-[(3aR,7aR)-4-(3-fluorophenyl)-hexahydro-2H-pyrrolo[3,2-b]pyridin-1-yl]pyridin-4-yl}piperidin-4-yl)ethanol (300 mg, 0.707 mmol, Intermediate SA) in DCM (5 mL) was added Dess-Martin periodinane (899.11 mg, 2.121 mmol) in portions at 0° C. under air atmosphere. The resulting mixture was stirred for 1 h at rt under air atmosphere. On completion, the mixture was concentrated under reduced pressure. The residue was purified by reverse-phase flash chromatography (column, C18 silica gel; mobile phase, MeCN in Water (0.1% TFA), 10% to 30% gradient in 60 min; detector, UV 254 nm) to give the title compound (180 mg, 60% yield) as a brown oil. LC/MS (ESI, m/z): [(M+H)]-=423.2.

### 6-(1-(3-(1H-1,2,3-triazol-1-yl) propanoyl)-1,2,5,6-tetrahydropyridin-3-yl)-7-fluoro-4-(2-methoxy-4-(4-(3-(methylamino) propyl) piperazin-1-yl) phenyl)-N,N-dimethyl-1H-indole-2-carboxamide hydrochloride (Intermediate RY)

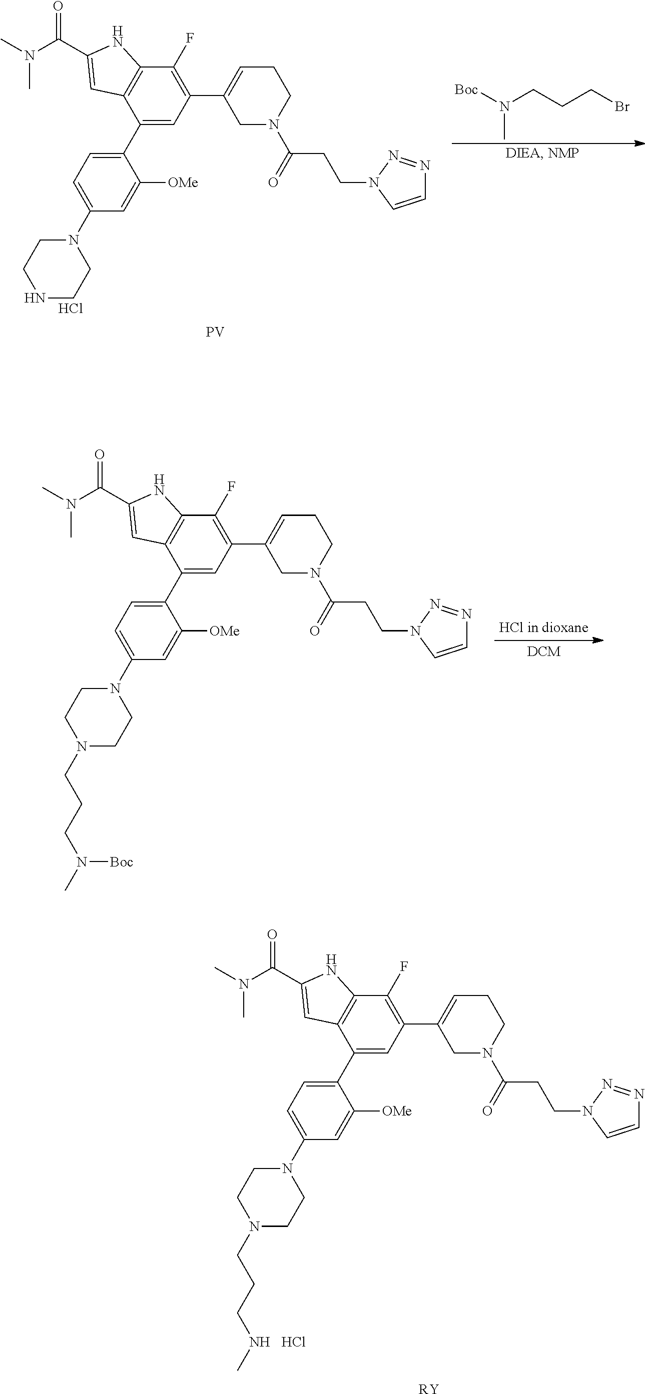

### Step 1—Tert-butyl (3-(4-(4-(6-(1-(3-(1H-1,2,3-triazol-1-yl)propanoyl)-1,2,5,6-tetrahydropyridin-3-yl)-2-(dimethylcarbamoyl)-7-fluoro-1H-indol-4-yl)-3-methoxyphenyl)piperazin-1-yl)propyl)(methyl)carbamate

To a stirred mixture of 7-fluoro-4-[2-methoxy-4-(piperazin-1-yl)phenyl]-N,N-dimethyl-6-{1-[3-(1,2,3-triazol-1-yl)propanoyl]-5,6-dihydro-2H-pyridin-3-yl}-1H-indole-2-carboxamide hydrochloride (200 mg, 0.314 mmol, Intermediate PV) and tert-butyl N-(3-bromopropyl)-N-methylcarbamate (120 mg, 0.476 mmol, CAS #828272-19-1) in NMP (2 mL) was added DIEA (120 mg, 0.928 mmol) dropwise at rt. The resulting mixture was stirred for 2 h at 110° C. On completion, the mixture was cooled to rt and purified by reverse phase flash chromatography (Column: WelFlash™ C18-I, 20-40 m, 120 g; Eluent A: Water (plus 10 mmol/L NH₄HCO₃); Eluent B: ACN; Gradient: 55%-75% B in 25 min: Flow rate: 60 mL/min; Detector: 220/254 nm; desired fractions were collected at 65% B and concentrated under reduced pressure) to give the title compound (110 mg, 45% yield) as a yellow solid. LC/MS (ESI, m/z): [(M+H)]⁺=772.2.

### Step 2—6-(1-(3-(1H-1,2,3-triazol-1-yl) propanoyl)-1,2,5,6-tetrahydropyridin-3-yl)-7-fluoro-4-(2-methoxy-4-(4-(3-(methylamino) propyl) piperazin-1-yl) phenyl)-N, N-dimethyl-1H-indole-2-carboxamide hydrochloride

To a stirred mixture of tert-butyl N-[3-(4-{4-[2-(dimethylcarbamoyl)-7-fluoro-6-{1-[3-(1,2,3-triazol-1-yl)propanoyl]-5,6-dihydro-2H-pyridin-3-yl}-1H-indol-4-yl]-3-methoxyphenyl}piperazin-1-yl)propyl]-N-methylcarbamate (110 mg, 0.142 mmol) in DCM (1 mL) was added HCl (g) (1 mL, 4 mmol) dropwise at rt. The resulting mixture was stirred for 1 h at rt. On completion, the mixture was concentrated under reduced pressure to give the title compound (100 mg) as a yellow solid. LC/MS (ESI, m/z): [(M+H)]⁺=672.3.

### 2-[1-(2-Bromopyridin-4-yl)piperidin-4-yl]ethanol (Intermediate RZ)

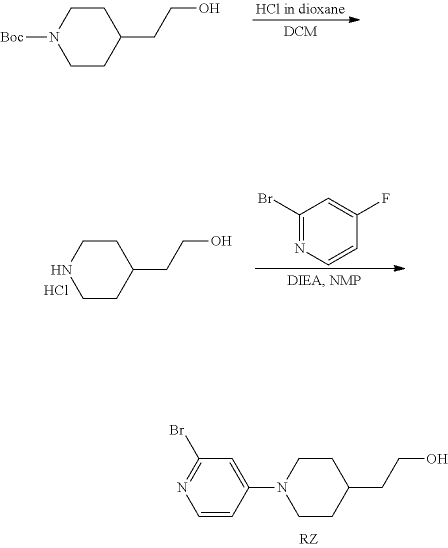 [SMILES: CC(C)(C)OC(=O)N1CCC(CCO)CC1 ; Cl ; OCCC1CCNCC1 ; Fc1ccnc(Br)c1 ; OCCC1CCN(c2ccnc(Br)c2)CC1]

### Step 1—4-Piperidine ethanol hydrochloride

To a stirred solution of tert-butyl 4-(2-hydroxyethyl)piperidine-1-carboxylate (28 g, 120 mmol, CAS #89151-44-0) in DCM (150 mL) was added HCl (gas) in 1,4-dioxane (150 mL) at rt. The resulting mixture was stirred for 2 h at rt. On completion, the mixture was concentrated under vacuum to afford the title compound (20 g, 99% yield) as a light yellow oil. LC/MS (ESI, m/z): [(M+H)]⁺=130.2.

### Step 2—2-[1-(2-Bromopyridin-4-yl)piperidin-4-yl]ethanol

To a stirred mixture of 4-piperidineethanol hydrochloride (20 g, 120 mmol) and 2-bromo-4-fluoropyridine (25.50 g, 144.9 mmol, CAS #357927-50-5) in NMP (200 mL) was added DIEA (63.09 mL, 362.2 mmol) at 0° C. The resulting mixture was stirred for 1 h at 130° C. On completion, the mixture was cooled to rt and diluted with water (500 mL). The resulting mixture was extracted with EtOAc (2×800 mL). The combined organic layers were washed with brine (3×300 mL), and dried over anhydrous Na₂SO₄. After filtration, the filtrate was concentrated under reduced pressure. The residue was purified by trituration with hexane (200 mL). The resulting mixture was filtered, the filter cake was washed with Et₂O (3×50 mL) to afford the title compound (22 g, 64% yield) as a white solid. LC/MS (ESI, m/z): [(M+H)]⁺=285.2, LC/MS (ESI, m/z): [(M+H+2)]⁺=287.2.

### 2-(1-{2-[(3aR,7aR)-4-(3-fluorophenyl)-hexahydro-2H-pyrrolo[3,2-b]pyridin-1-yl]pyridin-4-yl}piperidin-4-yl)ethanol (Intermediate SA)

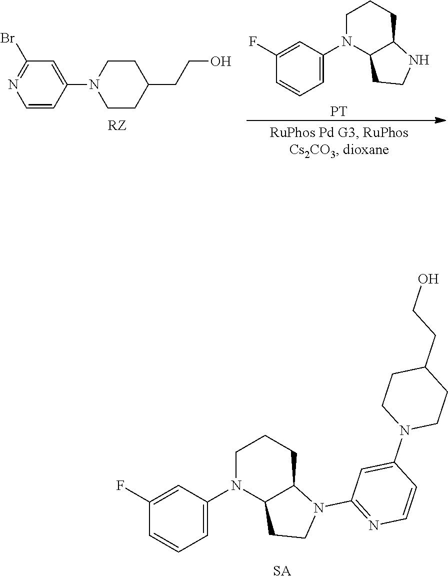

To a stirred mixture of 2-[1-(2-bromopyridin-4-yl)piperidin-4-yl]ethanol (800 mg, 2.81 mmol, Intermediate RZ) and (3aR,7aR)-4-(3-fluorophenyl)-octahydropyrrolo[3,2-b]pyridine (494.37 mg, 2.244 mmol, Intermediate PT) in dioxane (10 mL) were added Pd-PEPPSI-IPentCl (235.96 mg, 0.281 mmol) and Cs₂CO₃ (2.74 g, 8.42 mmol) at rt. The resulting mixture was stirred for 16 h at 100° C. under nitrogen atmosphere. On completion, the mixture was cooled to rt and concentrated under reduced pressure. The residue was purified by reverse phase flash chromatography (Column: WelFlash™ C18-I, 20-40 um, 330 g: Eluent A: Water (plus 10 mmol/L TFA); Eluent B: ACN; Gradient: 25%-60% B in 25 min; Flow rate: 90 mL/min; Detector: 220 nm: desired fractions were collected at 48% B and concentrated under reduced pressure) to afford the title compound (650 mg, 55% yield) as a brown solid. LC/MS (ESI, m/z): [(M+H)]⁻=425.2.

### 2-[(3aR,7aR)-4-(3-fluorophenyl)-hexahydro-2H-pyrrolo[3,2-b]pyridin-1-yl]-4-[4-(2-{12-[2-(2-iodoethoxy)ethoxy]ethoxy}ethyl)piperidin-1-yl]pyridine (Intermediate SB)

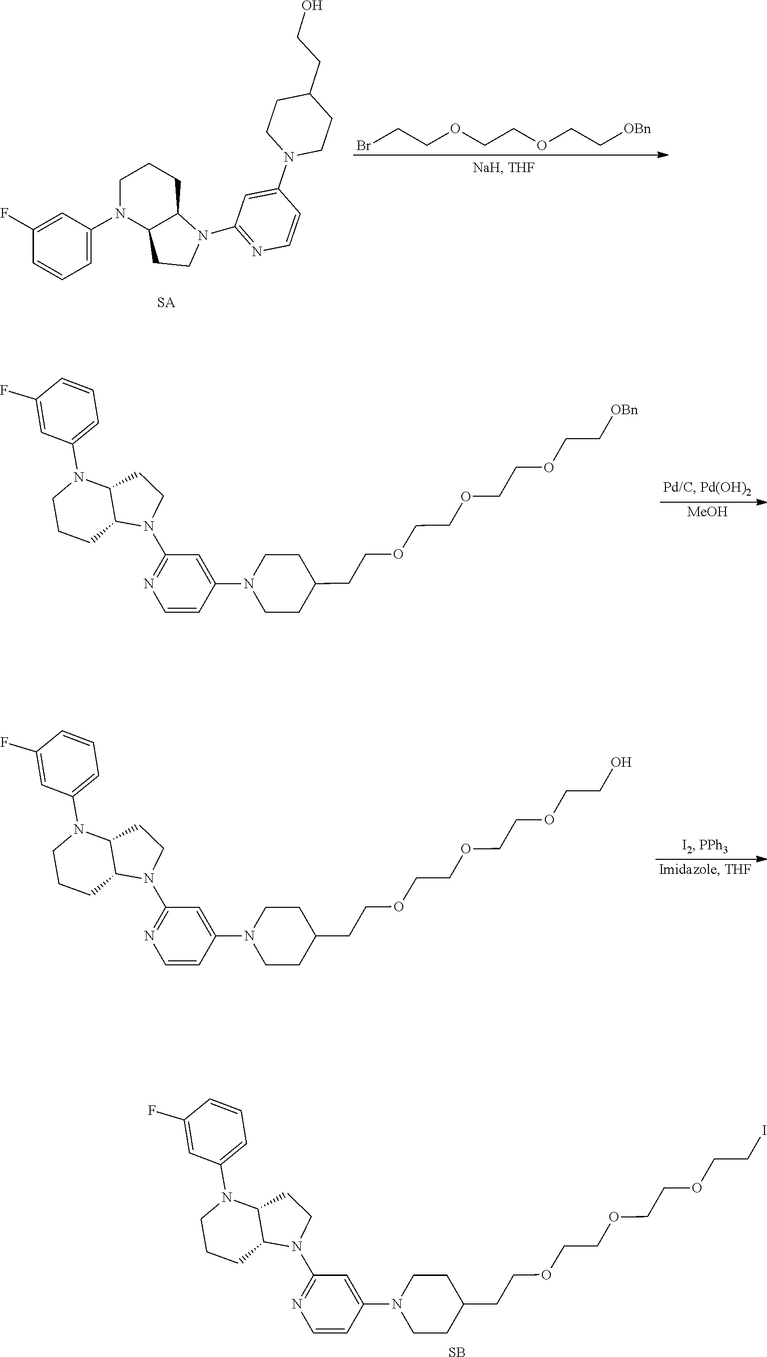

### Step 1—2-[(3aR,7aR)-4-(3-fluorophenyl)-hexahydro-2H-pyrrolo[3,2-b]pyridin-1-yl]-4-[4-(1-phenyl-2,5,8,11-tetraoxatridecan-13-yl)piperidin-1-yl]pyridine

To a stirred mixture of 2-(1-{2-[(3aR,7aR)-4-(3-fluorophenyl)-hexahydro-2H-pyrrolo[3,2-b]pyri din-1l-yl]pyridin-4-yl}piperidin-4-yl)ethanol (600 mg, 1.41 mmol, Intermediate SA) and ({2-[2-(2-bromoethoxy)ethoxy]ethoxy-}methyl)benzene (642.72 mg, 2.119 mmol, CAS #125562-32-5) in THF (20 mL) was added NaH (84.79 mg, 2.119 mmol, 60% dispersion in mineral oil) at 0° C. The resulting mixture was stirred for 16 h at 70° C. On completion, the mixture was cooled to rt and diluted with water (50 mL). The resulting mixture was extracted with EtOAc (3×50 mL). The combined organic layers were washed with brine (2×50 mL), and dried over anhydrous Na₂SO₄. After filtration, the filtrate was concentrated under reduced pressure. The residue was purified by reverse phase flash chromatography (Column: WelFlash™ C18-I, 20-40 um, 330 g; Eluent A: Water (plus 10 mmol/L FA); Eluent B: ACN; Gradient: 25%-60% B in 25 min; Flow rate: 60 mL/min; Detector: 220 nm; desired fractions were collected at 58% B and concentrated under reduced pressure) to afford the title compound (800 mg, 88% yield) as a light yellow oil. LC/MS (ESI, m/z): [(M+H)]⁺=647.2.

### Step 2—2-(2-{2-[2-(1-{2-[(3aR,7aR)-4-(3-fluorophenyl)-hexahydro-2H-pyrrolo[3,2-b]pyridin-1-yl]pyridin-4-yl}piperidin-4-yl)ethoxy]ethoxy}ethoxy)ethanol

To a solution of 2-[(3aR,7aR)-4-(3-fluorophenyl)-hexahydro-2H-pyrrolo[3,2-b]pyridin-1-yl]-4-[4-(1-phenyl-2,5,8,11-tetraoxatridecan-13-yl)piperidin-1-yl]pyridine (800 mg, 1.24 mmol) in MeOH (10 mL) were added Pd/C (131.62 mg, 1.237 mmol) and Pd(OH)₂/C (173.68 mg, 1.237 mmol) under nitrogen atmosphere. The reaction system was degassed under vacuum and purged with H₂ several times, then it was hydrogenated under H₂ balloon (˜1 atm) at rt for 1 h. After completion of the reaction, Pd/C and Pd(OH)₂/C were filtered off through celite. The filter cake was washed with MeOH (3×100 mL). The corresponding filtrate was concentrated under reduced pressure to afford the title compound (300 mg, 44% yield) as a light brown oil. LC/MS (ESI, m/z): [(M+H)]⁺=557.2.

### Step 3—2-[(3aR,7aR)-4-(3-fluorophenyl)-hexahydro-2H-pyrrolo[3,2-b]pyridin-1-yl]-4-[4-(2-{2-[2-(2-iodoethoxy)ethoxy]ethoxy}ethyl)piperidin-1-yl]pyridine

To a stirred mixture of 2-(2-{2-[2-(1-{2-[(3aR,7aR)-4-(3-fluorophenyl)-hexahydro-2H-pyrrolo[3,2-b]pyridin-1-yl]pyridin-4-yl}piperidin-4-yl)ethoxy]ethoxy}ethoxy)ethanol (280 mg, 0.503 mmol) and PPh₃ (263.84 mg, 1.006 mmol) in THF (6 mL) was added I₂ (191.48 mg, 0.754 mmol) at rt. The resulting mixture was stirred for 2 h at rt. On completion, the mixture was concentrated under reduced pressure. The residue was purified by silica gel column chromatography, eluted with CH₂Cl₂/MeOH (10:1), to afford the title compound (300 mg, 90% yield) as a light brown oil. LC/MS (ESI, m/z): [(M+H)]⁺=667.2.

### 2-Bromo-4-[2-(1-phenyl-2,5,8,11,14-pentaoxahexadecan-16-yl)piperidin-1-yl]pyridine (Intermediate SC)

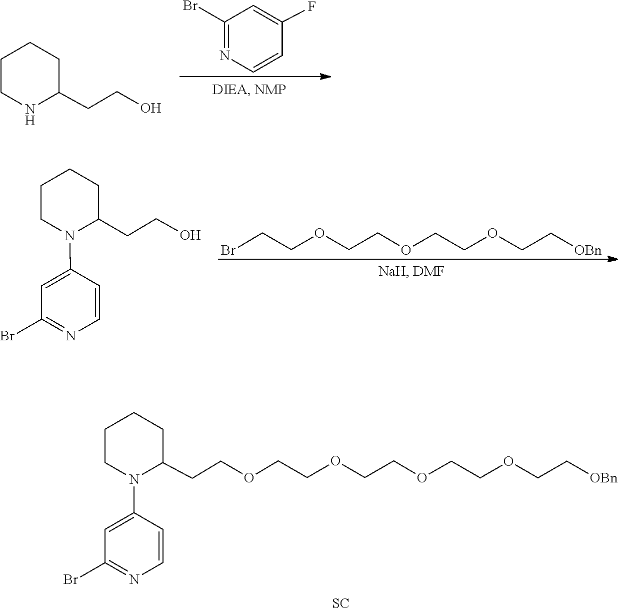 [SMILES: OCCC1CCCCN1 ; Fc1ccnc(Br)c1 ; OCCC1CCCCN1c1ccnc(Br)c1 ; BrCCOCCOCCOCCOCc1ccccc1 ; Brc1cc(N2CCCCC2CCOCCOCCOCCOCCOCc2ccccc2)ccn1 ; "SC"]

### Step 1—2-[1-(2-Bromopyridin-4-yl)piperidin-2-yl]ethanol

To a stirred solution of 2-piperidine ethanol (5 g, 40 mmol, CAS #1484-84-0) and DIEA (13.48 mL, 77.40 mmol) in NMP (50 mL) was added 2-bromo-4-fluoropyridine (8.17 g, 46.4 mmol, CAS #357927-50-5) at rt. The resulting mixture was stirred for 16 h at 120° C. On completion, the mixture was cooled to rt and water (100 mL) was added. The aqueous layer was extracted with EtOAc (3×40 mL). The resulting mixture was concentrated under reduced pressure. The residue was purified by silica gel column chromatography, eluted with CH₂Cl₂/MeOH (10:1), to afford the title compound (10 g, 83% yield) as a yellow oil. ¹H NMR (300 MHz, Chloroform-d) δ 7.82 (d, J=6.1 Hz, 1H), 6.81 (d, J=2.5 Hz, 1H), 6.63 (dd, J=6.2, 2.5 Hz, 1H), 4.29-4.19 (m, 1H), 3.66-3.59 (m, 2H), 3.59-3.54 (m, 1H), 3.04-2.89 (m, 1H), 1.94-1.76 (m, 2H), 1.75-1.44 (m, 6H); LC/MS (ESI, m/z): [(M+H)]⁺=285.0, LC/MS (ESI, m/z): [(M+H)]⁺2=287.0.

### Step 2—2-Bromo-4-[2-(1-phenyl-2,5,8,11,14-pentaoxahexadecan-16-yl)piperidin-1-yl]pyridine

To a stirred solution of 2-[1-(2-bromopyridin-4-yl)piperidin-2-yl]ethanol (5 g, 20 mmol) in DMF (40 mL) was added NaH (631.12 mg, 26.298 mmol, 60% dispersion in mineral oil) at 0° C. The resulting mixture was stirred for 30 min at 0° C. To the above mixture was added 13-bromo-1-phenyl-2,5,8,11-tetraoxatridecane (9.13 g, 26.3 mmol) at 0° C. The resulting mixture was stirred for an additional 16 h at 50° C. On completion, the mixture was cooled to rt and quenched with sat. NH₄Cl (aq.) at 0° C. The aqueous layer was extracted with EtOAc (3×50 mL). The resulting mixture was concentrated under reduced pressure. The residue was purified by silica gel column chromatography, eluted with CH₂Cl₂/MeOH (6:1), to afford the title compound (5 g, 52% yield) as a brown oil. ¹H NMR (300 MHz, Chloroform-d) δ 7.91 (d, J=6.1 Hz, 1H), 7.34-7.25 (m, 5H), 6.84 (d, J=2.5 Hz, 1H), 6.64 (dd, J=6.2, 2.5 Hz, 1H), 4.57 (s, 2H), 4.28-4.14 (m, 1H), 3.81 (t, J=6.3 Hz, 2H), 3.67-3.58 (m, 12H), 3.53-3.46 (m, 4H), 3.37-3.27 (m, 1H), 3.04-2.89 (m, 1H), 2.05-1.93 (m, 1H), 1.89-1.75 (m, 1H), 1.72-1.45 (m, 6H); LC/MS (ESI, m/z): [(M+H)]⁺=551.2, LC/MS (ESI, m/z): [(M+H)]⁺+2=553.2.

### 2-[(3aR,7aR)-4-(3-fluorophenyl)-hexahydro-2H-pyrrolo[3,2-b]pyridin-1-yl]-4-[2-(14-iodo-3,6,9,12-tetraoxatetradecan-1-yl)piperidin-1-yl]pyridine (Intermediate SD)

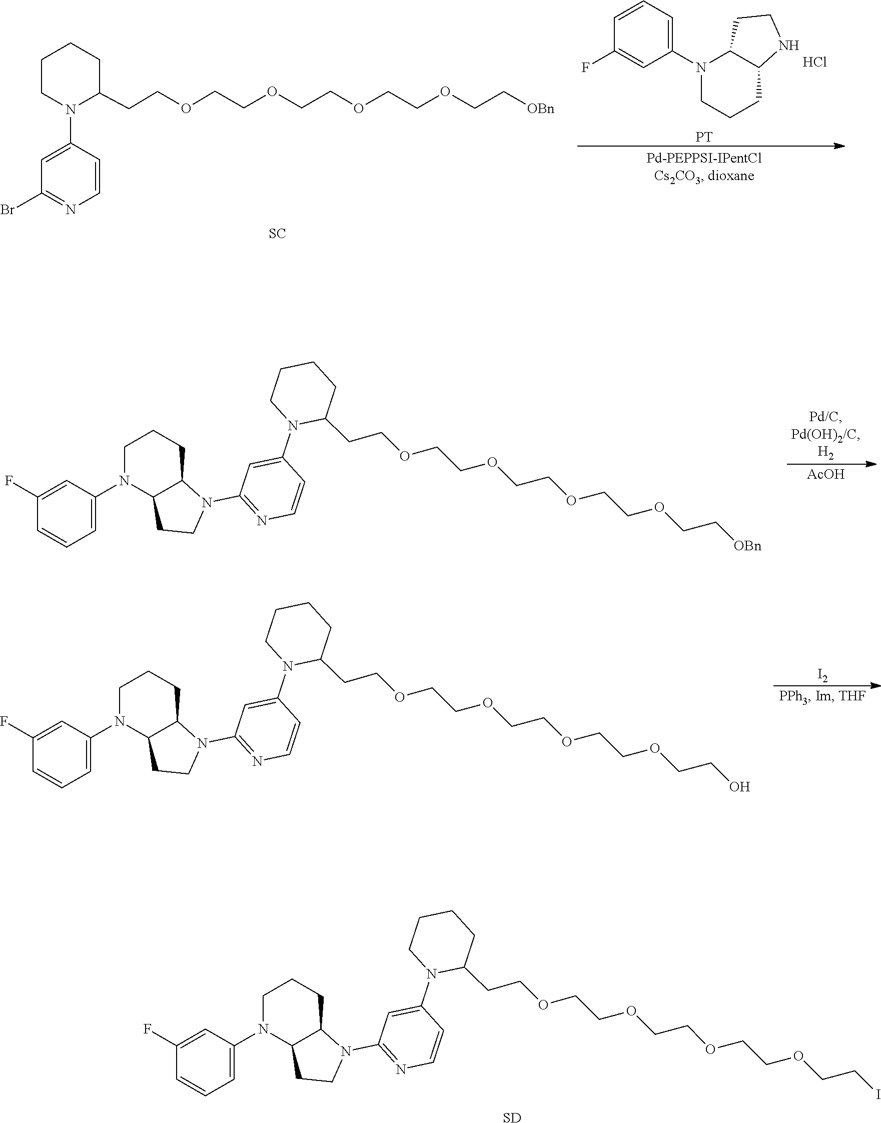

### Step 1—2-[(3aR,7aR)-4-(3-fluorophenyl)-hexahydro-2H-pyrrolo[3,2-b]pyridin-1-yl]-4-[2-(1-phenyl-2,5,8,11,14-pentaoxahexadecan-16-yl)piperidin-1-yl]pyridine

To a stirred solution of (3aR,7aR)-4-(3-fluorophenyl)-octahydropyrrolo[3,2-b]pyridine hydrochloride (500 mg, 1.95 mmol, Intermediate PT) and Cs₂CO₃ (1.90 g, 5.84 mmol) in 1,4-dioxane (10 mL) were added 2-bromo-4-[2-(1-phenyl-2,5,8,11,14-pentaoxahexadecan-16-yl)piperidin-1-yl]pyridine (1.29 g, 2.34 mmol, Intermediate SC) and Pd-PEPPSI-IPentCl (163.81 mg, 0.195 mmol) at rt. The resulting mixture was stirred for 2 h at 100° C. under nitrogen atmosphere. On completion, the mixture was cooled to rt and concentrated under reduced pressure. The residue was purified by silica gel column chromatography, eluted with CH₂Cl₂/MeOH (10:1), to afford the title compound (600 mg, 45% yield) as a brown yellow oil. ¹H NMR (400 MHz, Chloroform-d) δ 7.85 (d, J=6.8 Hz, 1H), 7.38-7.34 (m, 5H), 7.31-7.28 (m, 2H), 7.23-7.16 (m, 1H), 6.71-6.65 (m, 1H), 6.63-6.56 (m, 1H), 6.53-6.46 (m, 1H), 4.58 (s, 2H), 4.50-4.41 (m, 1H), 4.33-4.21 (m, 1H), 3.72-3.69 (m, 2H), 3.69-3.65 (m, 12H), 3.65-3.61 (m, 3H), 3.56-3.49 (m, 4H), 3.46-3.32 (m, 2H), 3.08-2.98 (m, 1H), 2.94-2.83 (m, 1H), 2.50-2.41 (m, 1H), 2.23-2.11 (m, 1H), 2.07-2.01 (m, 2H), 1.91-1.83 (m, 2H), 1.75-1.57 (m, 8H); LC/MS (ESI, m/z): [(M+H)]⁺=691.5.

### Step 2—14-(1-{2-[(3aR,7aR)-4-(3-fluorophenyl)-hexahydro-2H-pyrrolo[3,2-b]pyridin-1-yl]pyridin-4-yl}piperidin-2-yl)-3,6,9,12-tetraoxatetradecan-1-ol

To a solution of 2-[(3aR,7aR)-4-(3-fluorophenyl)-hexahydro-2H-pyrrolo[3,2-b]pyridin-1-yl]-4-[2-(1-phenyl-2,5,8,11,14-pentaoxahexadecan-16-yl)piperidin-1-yl]pyridine (600 mg, 0.868 mmol) and CH₃COOH (0.5 ml) in 15 mL MeOH were added Pd/C (10 wt %, 60 mg) and Pd(OH)₂/C (10 wt %, 60 mg) under nitrogen atmosphere. The reaction system was degassed under vacuum and purged with H₂ several times, then it was hydrogenated under H₂ balloon (˜1 atm) at 25° C. for 6 h. After completion of the reaction, Pd/C and Pd(OH)₂/C were filtered off through celite. The filter cake was washed with MeOH (3×10 mL). The corresponding filtrate was concentrated under reduced pressure. The residue was purified by reverse phase flash chromatography (Column: WelFlash™ C18-I, 20-40 um, 120 g; Eluent A: Water (plus 10 mmol/L NH₄HCO₃); Eluent B: ACN; Gradient: 60%-95% B in 30 min; Flow rate: 80 mL/min; Detector: 220/254 nm; desired fractions were collected at 82% B and concentrated under reduced pressure) to afford the title compound (100 mg, 22% yield) as a white oil. ¹H NMR (400 MHz, DMSO-d₆) δ 7.69 (d, J=6.0 Hz, 1H), 7.24-7.15 (m, 1H), 6.82-6.68 (m, 2H), 6.50-6.43 (m, 1H), 6.16-6.10 (m, 1H), 5.68-5.61 (m, 1H), 4.61-4.52 (m, 1H), 4.51-4.36 (m, 1H), 4.18-4.07 (m, 2H), 3.66-3.56 (m, 1H), 3.54-3.44 (m, 15H), 3.42-3.36 (m, 5H), 3.30-3.22 (m, 1H), 2.92-2.72 (m, 2H), 2.27-2.18 (m, 1H), 2.09-1.98 (m, 1H), 1.97-1.85 (m, 1H), 1.82-1.73 (m, 3H), 1.71-1.38 (in, 8H); LC/MS (ESI, m/z): [(M+H)]⁺=601.4.

### Step 3—2-[(3aR,7aR)-4-(3-fluorophenyl)-hexahydro-2H-pyrrolo[3,2-b]pyridin-1-yl]-4-[2-(14-iodo-3,6,9,12-tetraoxatetradecan-1-yl)piperidin-1-yl]pyridine

To a stirred solution of 14-(1-{2-[(3aR,7aR)-4-(3-fluorophenyl)-hexahydro-2H-pyrrolo[3,2-b]pyridin-1-yl]pyridin-4-yl}piperidin-2-yl)-3,6,9,12-tetraoxatetradecan-1-ol (100 mg, 0.166 mmol) and imidazole (28.33 mg, 0.415 mmol) in THF (4 mL) were added PPh₃ (109.15 mg, 0.415 mmol) and I₂ (105.62 mg, 0.415 mmol) at rt. The resulting mixture was stirred for 1 h at rt. On completion, the reaction was quenched with sat. Na₂S₂O₃ (aq.) at rt. The aqueous layer was extracted with EtOAc (3×10 mL). The resulting mixture was concentrated under reduced pressure. The residue was purified by reverse phase flash chromatography (Column: WelFlash™ C18-I, 20-40 um, 120 g; Eluent A: Water (plus 10 mmol/L NH₄HCO₃); Eluent B: MeOH; Gradient: 60%-95% B in 25 min; Flow rate: 80 mL/min; Detector: 220/254 nm; desired fractions were collected at 95% B and concentrated under reduced pressure) to afford the title compound (100 mg, 85% yield) as a light yellow oil. ¹H NMR (400 MHz, Chloroform-d) δ 7.90-7.83 (m, 1H), 7.27-7.25 (m, 1H), 7.23-7.16 (m, 1H), 6.70 (d, J=8.5 Hz, 1H), 6.61 (d, J=12.9 Hz, 1H), 6.55-6.46 (m, 1H), 5.66 (s, 1H), 4.53-4.40 (m, 1H), 4.37-4.16 (m, 2H), 3.76 (t, J=7.0 Hz, 2H), 3.71-3.64 (m, 10H), 3.60-3.39 (m, 8H), 3.27 (t, J=6.9 Hz, 2H), 3.05-2.83 (m, 2H), 2.20-1.95 (m, 4H), 1.90-1.79 (m, 2H), 1.76-1.59 (in, 8H); LC/MS (ESI, m/z): [(M+H)]⁺=711.2.

### 4-(3,6-Difluoro-2-methylphenyl)-5-[4-(6-hydroxyhexyl)benzoyl]-1-methylpyrrole-3-carboxylic acid (Intermediate SE)

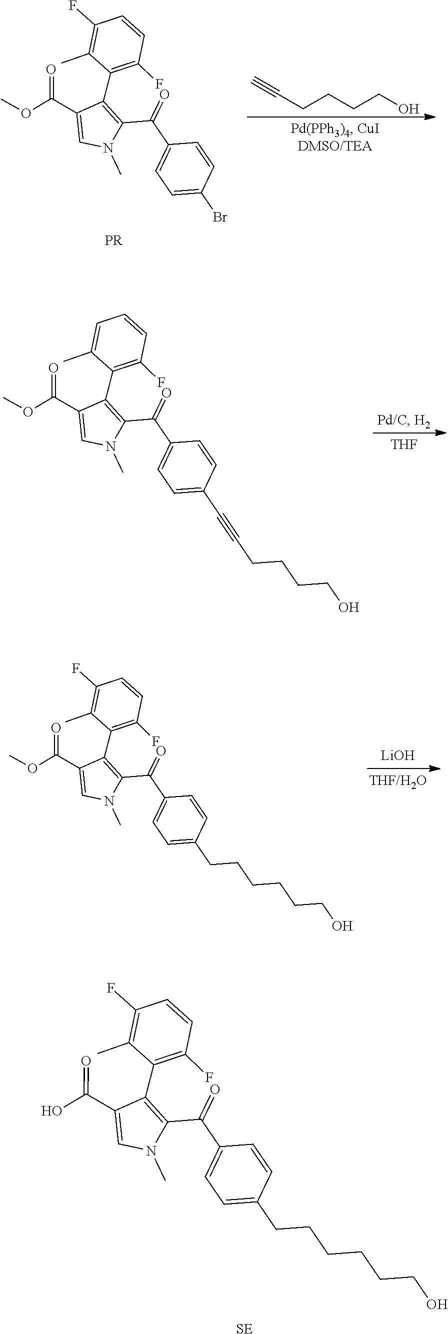

### Step 1—Methyl 4-(3,6-difluoro-2-methylphenyl)-5-[4-(6-hydroxyhex-1-yn-1-yl)benzoyl]1-methylpyrrole-3-carboxylate

To a stirred mixture of methyl 5-(4-bromobenzoyl)-4-(3,6-difluoro-2-methylphenyl)-1-methylpyrrole-3-carboxylate (450 mg, 1.00 mmol, Intermediate PR) and hex-5-yn-1-ol (197.05 mg. 2.008 mmol, CAS #928-90-5) in DMSO (3 mL) were added CuI (38.24 mg, 0.201 mmol) and TEA (1 mL) in portions at rt under air atmosphere. To the above mixture was added Pd(PPh₃)₄ (116.01 mg, 0.100 mmol) in portions over 3 min at rt. The resulting mixture was stirred for 2 h at 80° C. under nitrogen atmosphere. On completion, the mixture was cooled to rt and purified by reverse phase flash chromatography (Column: WelFlash™ C18-I, 20-40 um, 120 g; Eluent A: Water (plus 10 mmol/L NH₄HCO₃); Eluent B: ACN; Gradient: 25%-55% B in 25 min; Flow rate: 60 mL/min; Detector: 220/254 nm; desired fractions were collected at 43% B and concentrated under reduced pressure) to afford the title compound (390 mg, 84% yield) as a yellow oil. LC/MS (ESI, m/z): [(M+H₂O)]=466.3.

### Step 2—Methyl 4-(3,6-difluoro-2-methylphenyl)-5-(4-(6-hydroxyhexyl)benzoyl)-1-methyl-1H-pyrrole-3-carboxylate

To a stirred mixture of methyl 4-(3,6-difluoro-2-methylphenyl)-5-[4-(6-hydroxyhex-1-yn-1-yl)benzoyl]-1-methylpyrrole-3-carboxylate (370 mg, 0.795 mmol) in THF (10 mL) was added Pd/C (16.92 mg, 0.159 mmol) in portions at rt under nitrogen atmosphere. The resulting mixture was stirred for 0.5 h at rt under hydrogen atmosphere. On completion, the mixture was filtered, and the filter cake was washed with MeOH (3×5 mL). The filtrate was concentrated under reduced pressure to afford the title compound (350 mg, 94% yield) as a yellow solid. LC/MS (ESI, m/z): [(M+H)]⁺=470.2.

### Step 3—4-(3,6-Difluoro-2-methylphenyl)-5-[4-(6-hydroxyhexyl)benzoyl]-1-methylpyrrole-3-carboxylic acid

To a stirred mixture of methyl 4-(3,6-difluoro-2-methylphenyl)-5-[4-(6-hydroxyhexyl)benzoyl]-1-methylpyrrole-3-carboxylate (360 mg, 0.767 mmol) in MeOH (5 mL) was added LiOH·H₂O (5 mL) dropwise at it under air atmosphere. The resulting mixture was stirred for 1 h at 80° C. under air atmosphere. On completion, the mixture was cooled to rt and concentrated under reduced pressure. The resulting mixture was diluted with water (10 mL). The mixture was acidified to pH 6 with cone. HCl. The resulting mixture was extracted with EtOAc (3×10 mL). The combined organic layers were washed with water (3×10 mL), and dried over anhydrous Na₂SO₄. After filtration, the filtrate was concentrated under reduced pressure to afford the title compound (340 mg, 97% yield) as a yellow solid. LC/MS (ESI, m/z): [(M+H)]⁺=484.3.

### N-[(1R)-1-(3-chlorophenyl)-2-methanesulfonylethyl]-4-(3,6-difluoro-2-methylphenyl)-5-[4-(6-hydroxyhexyl)benzoyl]-1-methylpyrrole-3-carboxamide (Intermediate SF)

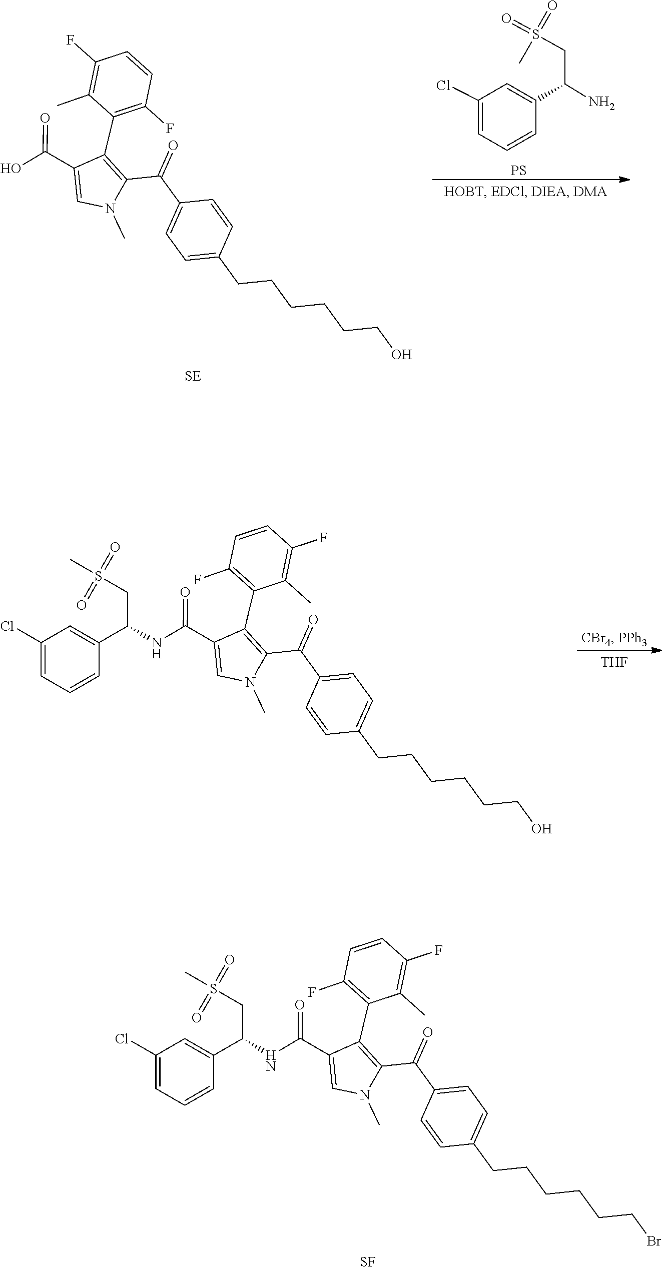

### Step 1—N-[(1R)-1-(3-chlorophenyl)-2-methanesulfonylethyl]-4-(3,6-difluoro-2-methylphenyl)-5-[4-(6-hydroxyhexyl)benzoyl]-1-methylpyrrole-3-carboxamide

To a stirred mixture of 4-(3,6-difluoro-2-methylphenyl)-5-[4-(6-hydroxyhexyl)benzoyl]-1-methylpyrrole-3-carboxylic acid (360 mg, 0.790 mmol, Intermediate SE) and EDCI (227.26 mg, 1.185 mmol) in DMA (4 mL) were added HOBT (160.19 mg, 1.185 mmol) and DIEA (306.45 mg, 2.370 mmol) in portions at it under air atmosphere. To the above mixture was added (IR)-1-(3-chlorophenyl)-2-methanesulfonylethanamine (184.71 mg, 0.790 mmol, Intermediate PS) in portions over 1 min at rt. The resulting mixture was stirred for 2 h at rt under air atmosphere. On completion, the mixture was purified by reverse phase flash chromatography (Column: WelFlash™ C18-I, 20-40 um, 120 g; Eluent A: Water (plus 10 mmol/L NH4HCO₃); Eluent B: ACN; Gradient: 25%-55% B in 25 min: Flow rate: 60 mL/min; Detector: 220/254 nm; desired fractions were collected at 47% B and concentrated under reduced pressure) to afford the title compound (390 mg. 74% yield) as a yellow solid. LC/MS (ESI, m/z): [(M+H)]⁺=671.2.

### Step 2—N-[(1R)-1-(3-chlorophenyl)-2-methanesulfonylethyl]-4-(3,6-difluoro-2-methylphenyl)-5-[4-(6-hydroxyhexyl)benzoyl]-1-methylpyrrole-3-carboxamide

To a stirred mixture of N-[(1R)-1-(3-chlorophenyl)-2-methanesulfonylethyl]-4-(3,6-difluoro-2-methylphenyl)-5-[4-(6-hydroxyhexyl)benzoyl]-1-methylpyrrole-3-carboxamide (340 mg, 0.507 mmol) and PPh₃ (265.73 mg, 1.014 mmol) in THF (15 mL) was added CBr₄ (839.94 mg. 2.535 mmol) in portions at rt under air atmosphere. The resulting mixture was stirred for 16 h at rt under air atmosphere. On completion, the mixture was purified by silica gel column chromatography, eluted with CH₂Cl₂/MeOH (10:1), to afford the title compound (100 mg, 27% yield) as a yellow solid. LC/MS (ESI, m/z): [(M+H)]⁻=755.2, 735.2.

### 4-(3,6-Difluoro-2-methylphenyl)-5-(4-hydroxybenzoyl)-1-methylpyrrole-3-carboxylic acid (Intermediate SG)

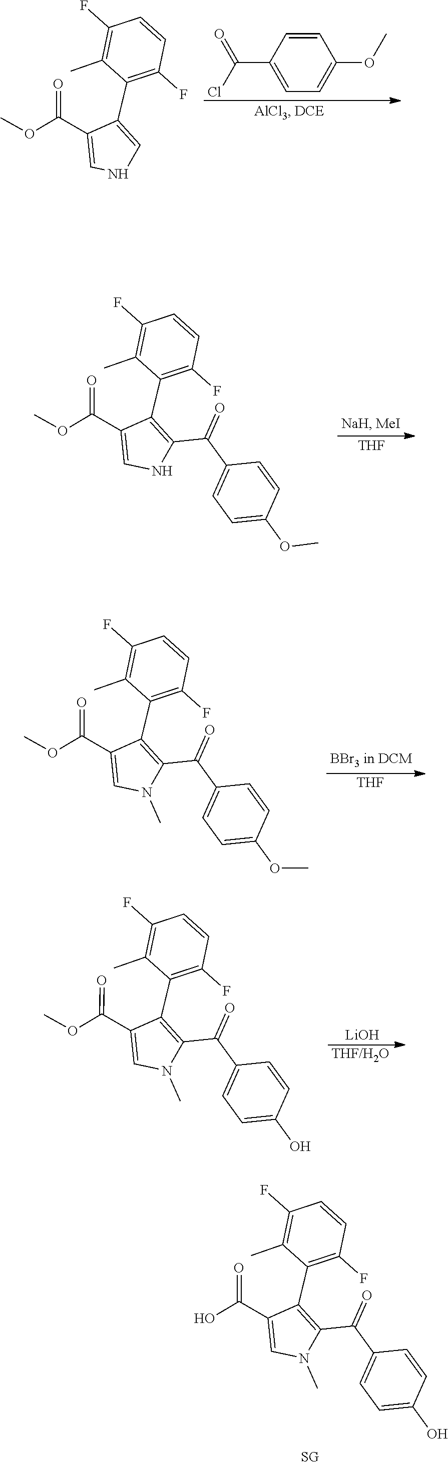

### Step 1—4-(3,6-Difluoro-2-methylphenyl)-5-(4-methoxybenzoyl)-1H-pyrrole-3-carboxylate

To a stirred solution of methyl 4-(3,6-difluoro-2-methylphenyl)-1H-pyrrole-3-carboxylate (15.00 g, 59.71 mmol, synthesized via Steps 1-2 of Intermediate PR) and 4-methoxy-benzoyl chloride, (20.37 g, 119.4 mmol) in DCE (200 mL) was added AlCl₃ (15.92 g. 119.4 mmol) at rt. The resulting mixture was stirred for 16 h at rt. After completion, the resulting mixture was diluted with water (500 mL). The resulting mixture was extracted with EtOAc (3×300 mL). The combined organic layers were washed with brine (3×100 mL), and dried over anhydrous Na₂SO₄. After filtration, the filtrate was concentrated under reduced pressure. The residue was purified by silica gel column chromatography, eluted with PE/EA (1:1), to afford the title compound (12.10 g, 53% yield) as a light yellow solid. LC/MS (ESI, m/z): [(M+H)]⁺=386.3.

### Step 2—4-(3,6-Difluoro-2-methylphenyl)-5-(4-methoxybenzoyl)-1-methylpyrrole-3-carboxylate

A solution of methyl 4-(3,6-difluoro-2-methylphenyl)-5-(4-methoxybenzoyl)-1H-pyrrole-3-carboxylate (12.00 g, 31.14 mmol) and NaH (2.49 g, 62.3 mmol, 60% dispersion in mineral oil) in THF (150 mL) was stirred for 0.5 h at rt. To the above mixture was added CH₃I (11.05 g, 77.85 mmol) dropwise at 0° C. The resulting mixture was stirred for an additional 16 h at rt. After completion, the reaction was quenched with Water/Ice at rt. The resulting mixture was extracted with EtOAc (3×300 mL). The combined organic layers were washed with brine (3×100 mL), and dried over anhydrous Na₂SO₄. After filtration, the filtrate was concentrated under reduced pressure. The residue was purified by silica gel column chromatography, eluted with PE/EA (1:1), to afford the title compound (8.70 g, 70% yield) as a white solid. LC/MS (ESI, m/z): [(M+H)]⁺=400.1.

### Step 3—4-(3,6-Difluoro-2-methylphenyl)-5-(4-hydroxybenzoyl)-1-methylpyrrole-3-carboxylate

To a stirred mixture of methyl 4-(3,6-difluoro-2-methylphenyl)-5-(4-methoxybenzoyl)-1-methylpyrrole-3-carboxylate (2 g, 5 mmol) in DCM (30 mL) was added borontribromide (15.02 mL, 15.02 mmol) dropwise at rt under air atmosphere. The resulting mixture was stirred for 16 h at rt under air atmosphere. On completion, the reaction was quenched by the addition of water (30 mL) and the mixture was extracted with CH₂Cl₂ (3×30 mL). The combined organic layers were washed with water (3×30 mL), and dried over anhydrous Na₂SO₄. After filtration, the filtrate was concentrated under reduced pressure. The residue was dissolved in DMF (5 mL). The mixture was purified by reverse phase flash chromatography (Column: WelFlash™ C18-I, 20-40 um, 120 g: Eluent A: Water (plus 10 mmol/L FA); Eluent B: ACN; Gradient: 25%-55% B in 25 min; Flow rate: 60 mL/min: Detector: 220/254 nm: desired fractions were collected at 36% B and concentrated under reduced pressure) to afford the title compound (1.1 g, 57% yield) as a yellow solid. LC/MS (ESI, m/z): [(M+H−56)]⁺=386.2.

### Step 4—4-(3,6-difluoro-2-methylphenyl)-5-(4-hydroxybenzoyl)-1-methyl-1H-pyrrole-3-carboxylic acid

A solution of methyl 4-(3,6-difluoro-2-methylphenyl)-5-(4-hydroxybenzoyl)-1-methylpyrrole-3-carboxylate (300 mg, 0.778 mmol) and LiOH·H₂O (160 mg, 3.81 mmol) in THF/H₂O (8 mL) was stirred for 1 h at rt under air atmosphere. On completion, the mixture was acidified to pH 6 with cone. HCl. The resulting mixture was extracted with EtOAc (3×10 mL). The combined organic layers were washed with water (1×5 mL), and dried over anhydrous MgSO₄. After filtration, the filtrate was concentrated under reduced pressure to give the title compound (160 mg, 55% yield) as an off-white solid. LC/MS (ESI, m/z): [(M−H)]⁻=370.1.

### N-[(1R)-1-(3-chlorophenyl)-2-methanesulfonylethyl]-4-(3,6-difluoro-2-methylphenyl)-5-(4-hydroxybenzoyl)-1-methylpyrrole-3-carboxamide (Intermediate SH)

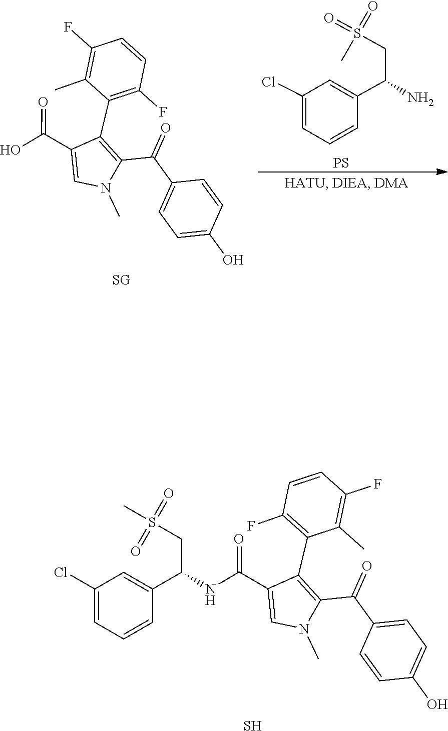

To a stirred mixture of 4-(3,6-difluoro-2-methylphenyl)-5-(4-hydroxybenzoyl)-1-methylpyrrole-3-carboxylic acid (1.1 g, 3.0 mmol, Intermediate SG) and (1R)-1-(3-chlorophenyl)-2-methanesulfonylethanamine (0.69 g, 2.96 mmol, Intermediate PS) in DMA (10 mL) was added DIEA (1.15 g, 8.89 mmol) and HATU (1.35 g, 3.55 mmol) dropwise at rt under air atmosphere. The resulting mixture was stirred for 16 h at rt under air atmosphere. On completion, the mixture was purified by reverse phase flash chromatography (Column: WelFlash™ C18-1, 20-40 um, 330 g: Eluent A: Water (plus 10 mmol/L NH4HCO3): Eluent B: ACN; Gradient: 25%-55% B in 25 min: Flow rate: 80 mL/min; Detector: 220/254 nm; desired fractions were collected at 36% B and concentrated under reduced pressure) to afford the title compound (500 mg, 29% yield) as a yellow solid. LC/MS (ESI, m/z): [(M+H)]⁺=587.2.

### N-[(1R)-1-(3-chlorophenyl)-2-methanesulfonylethyl]-4-(3,6-difluoro-2-methylphenyl)-5-(4-{2-[2-(2-hydroxyethoxy)ethoxy]ethoxy}benzoyl)-1-methylpyrrole-3-carboxamide (Intermediate SI)

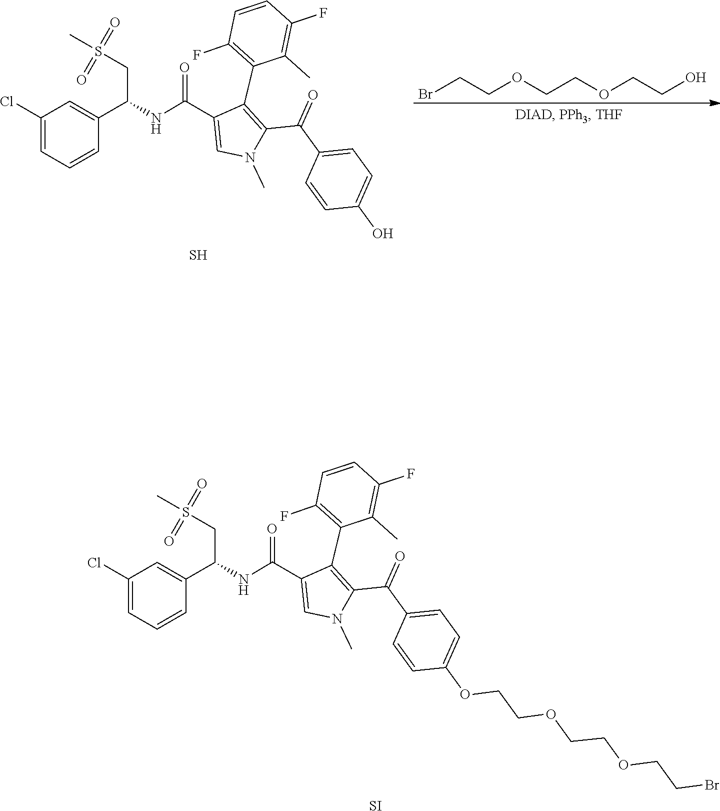

To a stirred mixture of 5-(4-{2-[2-(2-bromoethoxy)ethoxy]ethoxy}benzoyl)-N-[(1R)-1-(3-chlorophenyl)-2-methanesulfonylethyl]-4-(3,6-difluoro-2-methylphenyl)-1-methylpyrrole-3-carboxamide (300 mg, 0.384 mmol, Intermediate SH) and 2-(2-(2-bromoethoxy)ethoxy)ethan-1-ol (300 mg, 0.768 mmol, CAS #57641-67-5) in THF (8 mL) were added PPh₃ (21.88 mg, 0.084 mmol) and DEAD (14.53 mg, 0.084 mmol) in portions at 0° C. under air atmosphere. The resulting mixture was stirred for 16 h at rt under air atmosphere. On completion, the mixture was diluted with water (10 mL). The resulting mixture was extracted with EtOAc (3×20 mL). The combined organic layers were washed with water (3×20 mL), and dried over anhydrous Na₂SO₄. After filtration, the filtrate was concentrated under reduced pressure. The residue was purified by silica gel column chromatography, eluted with PE/EA (1:1), to afford the title compound (200 mg, 67% yield) as a yellow solid. LC/MS (ESI, m/z): [(M+H)]⁺=781.2, 783.2.

### 5-[4-(2-{2-[2-(2-Bromoethoxy)ethoxy]ethoxy}ethoxy)benzoyl]-N-[(1R)-1-(3-chlorophenyl)-2-methanesulfonylethyl]-4-(3,6-difluoro-2-methylphenyl)-1-methylpyrrole-3-carboxamide (Intermediate SJ)

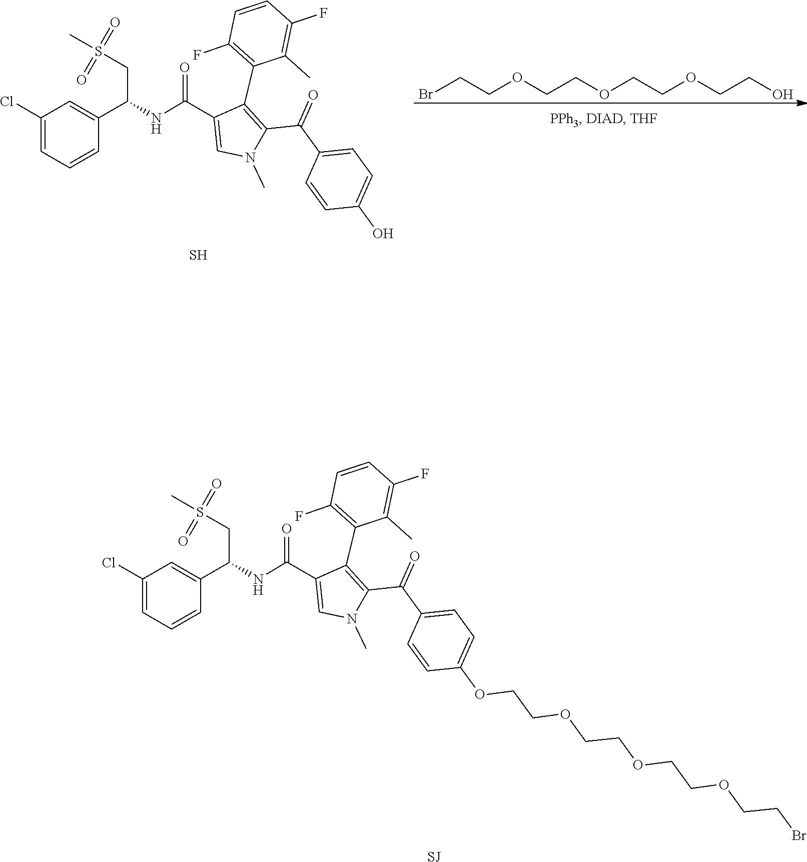

To a stirred solution of N-[(1R)-1-(3-chlorophenyl)-2-methanesulfonylethyl]-4-(3,6-difluoro-2-methylphenyl)-5-(4-hydroxybenzoyl)-1-methylpyrrole-3-carboxamide (300 mg, 0.511 mmol, Intermediate SH), PPh₃ (201 mg, 0.766 mmol) and 2-{2-[2-(2-bromoethoxy)ethoxy]ethoxy}ethanol (131 mg, 0.509 mmol, CAS #85141-94-2) in THF (5 mL) was added DIAD (155 mg, 0.767 mmol) dropwise at 0° C. The resulting mixture was stirred for 16 h at rt. On completion, the mixture was diluted with water (100 mL). The resulting mixture was extracted with EtOAc (3×100 mL). The combined organic layers were washed with brine (1×100 mL), and dried over anhydrous Na₂SO₄. After filtration, the filtrate was concentrated under reduced pressure. The residue was purified by silica gel column chromatography, eluted with PE/EA (1:1), to afford the title compound (300 mg, 71% yield) as a light yellow oil. LC/MS (ESI, m/z): [(M+H)]⁺=825.0.

### 4-(4-(4-Chloro-3-methylphenyl)piperidin-1-yl)-3,3-dimethylindolin-2-one (Intermediate SK)

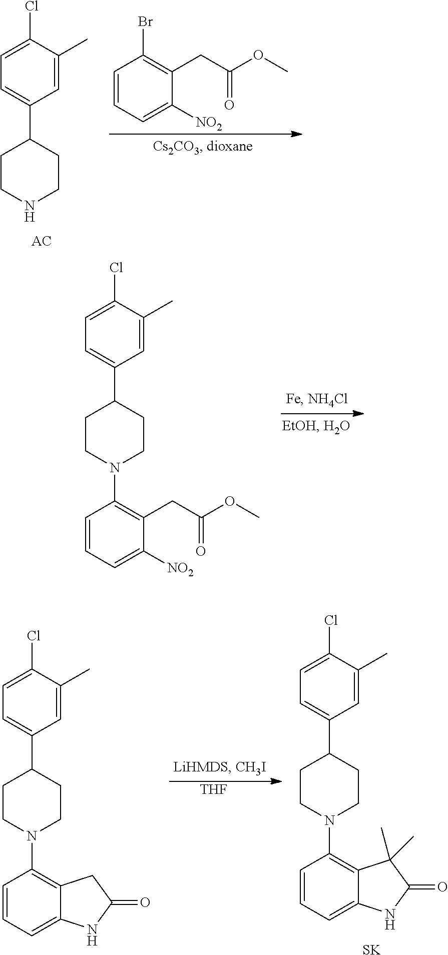

### Step 1—Methyl 2-(2-(4-(4-chloro-3-methylphenyl)piperidin-1-yl)-6-nitrophenyl)acetate

A mixture of methyl 2-(2-bromo-6-nitrophenyl)acetate (4.7 g, 17.2 mmol, CAS #266678-15-3), 4-(4-chloro-3-methylphenyl)piperidine (3.60 g, 17.2 mmol, Intermediate AC), Cs₂CO₃ (16.8 g, 51.5 mmol), 1,3-bis[2,6-bis(1-propylbutyl)phenyl]-4,5-dichloro-2H-imidazol-1-ium-2-ide; 3-chloropyridine dichloropalladium (1.67 g, 1.71 mmol) in dioxane (10 mL) was degassed and purged with N₂ three times. Then the mixture was stirred at 100° C. for 72 hrs under N₂ atmosphere. Upon completion, the reaction mixture was quenched with water (30 mL) and extracted with dichloromethane (3×30 mL). The combined organic layers were washed with aqueous NaCl (30 mL) and dried over anhydrous sodium sulfate, filtered and the filtrate was concentrated in vacuo to give the crude residue. The residue was purified by column chromatography (SiO₂, Petroleum ether:Ethyl acetate=10/1) to give the title compound (4.3 g, 44% yield) as a yellow solid. LC-MS (ESI⁺) m/z 403.1 (M+H)⁺.

### Step 2—4-(4-(4-Chloro-3-methylphenyl)piperidin-1-v-yl) indolin-2-one

To a solution of methyl 2-(2-(4-(4-chloro-3-methylphenyl)piperidin-1-yl)-6-nitrophenyl)acetate (4.3 g, 7.5 mmol) in EtOH (30 mL) and H₂O (15 mL) was added Fe (2.92 g, 52.3 mmol) and NH4Cl (2.80 g, 52.3 mmol). The mixture was then stirred at 60° C. for 3 hrs. Upon completion, the reaction mixture was filtered and the filtrate was concentrated in vacuo to give the crude residue, and the residue was extracted with dichloromethane (3×30 mL). The combined organic layers were washed with aqueous NaCl (30 mL) and dried over anhydrous sodium sulfate, filtered and the filtrate was concentrated in vacuo to give the crude residue. The crude product was triturated with PE:EA=10:1 at 25° C. for 0.3 hr to give the title compound (2.7 g, 81% yield) as a white solid. LC-MS (ESI⁺) m/z 341.0 (M+H)⁺; ¹H NMR (400 MHz, DMSO-d6) δ=10.35-10.07 (m, 1H), 7.38-7.26 (m, 2H), 7.16-7.05 (m, 2H), 6.64-6.54 (m, 1H), 6.50-6.42 (m, 1H), 3.51-3.45 (m, 2H), 3.43 (s, 2H), 2.80-2.71 (m, 2H), 2.68-2.59 (m, 1H), 2.32 (s, 3H), 1.88-1.69 (in, 4H).

### Step 3—4-(4-(4-Chloro-3-methylphenyl)piperidin-1-yl)-3,3-dimethylindolin-2-one

A mixture of 4-(4-(4-chloro-3-methylphenyl)piperidin-1-yl) indolin-2-one (890 mg, 2.61 mmol), CH₃I (927 mg, 6.53 mmol, 406 L), and LiHMDS (1 M, 10.44 mL) in THF (5 mL) was degassed and purged with N₂ three times. Then the mixture was stirred at 0° C. for 3 hrs under N₂ atmosphere. Upon completion, the reaction mixture was quenched/diluted with water (10 mL) and extracted with ethyl acetate (2×20 mL). The crude product was triturated with PE:EA=3:1 at 25° C. for 0.2 hrs to give the title compound (500 mg) as a white solid. LC-MS (EST*) m/z 369.1 (M+H)⁺; ¹H NMR (400 MHz, DMSO-d6) δ=10.38-10.20 (m, 1H), 7.40-7.26 (m, 2H), 7.21-7.11 (m, 2H), 7.01-6.93 (m, 1H), 6.73-6.64 (m, 1H), 2.98-2.82 (m, 4H), 2.69-2.59 (m, 1H), 2.33 (s, 3H), 2.00-1.97 (m, 1H), 1.88-1.71 (m, 4H), 1.58-1.53 (m, 1H), 1.43-1.35 (m, 6H), 1.22 (s, 1H), 1.19-1.14 (m, 1H).

### 3-(4-(4-(4-Chloro-3-methylphenyl)piperidin-1-yl)-3,3-dimethyl-2-oxoindolin-1-yl)piperidine-2,6-dione (Intermediate SL)

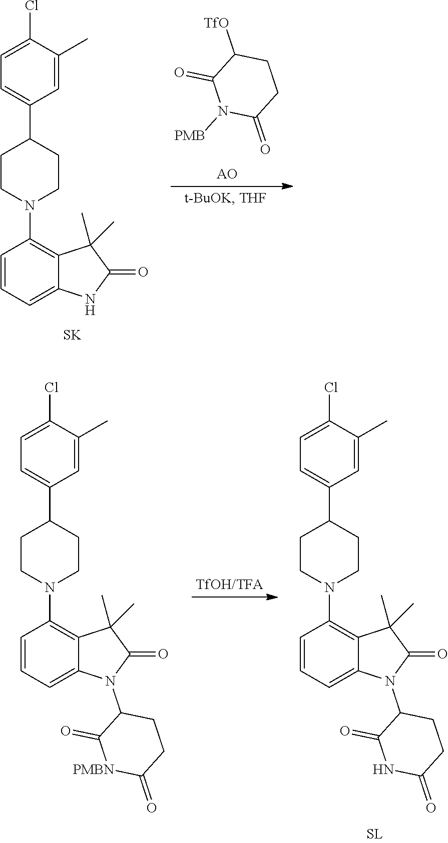

### Step 1—3-(4-(4-(4-Chloro-3-methylphenyl)piperidin-1-yl)-3,3-dimethyl-2-oxoindolin-1-yl)-1-(4-methoxybenzyl)piperidine-2,6-dione

A mixture of 4-(4-(4-chloro-3-methylphenyl)piperidin-1-yl)-3,3-dimethylindolin-2-one (500 mg, 949 μmol, Intermediate SK) and t-BuOK (213 mg, 1.90 mmol) in THE (2 mL) was degassed and purged with N₂ three times. Then the mixture was stirred at 25° C. for 1 hr under N₂ atmosphere, and 1-(4-methoxybenzyl)-2,6-dioxopiperidin-3-yl trifluoromethanesulfonate (543 mg, 1.42 mmol, Intermediate AO) in THF (2 mL) was added at 0° C. Then the mixture was stirred at 25° C. for 3 hrs under N₂ atmosphere. Upon completion, the reaction mixture was quenched with NH₄Cl and extracted with dichloromethane (3×30 mL). The combined organic layers were washed with aqueous NaCl (30 mL) and dried over anhydrous sodium sulfate, filtered and the filtrate was concentrated in vacuo to give the crude residue. The residue was purified by column chromatography (SiO₂, Petroleum ether:Ethyl acetate=6/1) to give the title compound (500 mg, 35% yield) as a white oil. LC-MS (ESI⁺) m/z 600.3 (M+H)⁺.

### Step 2—3-(4-(4-(4-Chloro-3-methylphenyl)piperidin-1-yl)-3,3-dimethyl-2-oxoindolin-1-yl)piperidine-2,6-dione

A mixture of 3-(4-(4-(4-chloro-3-methylphenyl)piperidin-1-yl)-3,3-dimethyl-2-oxoindolin-1-yl)-1-(4-methoxybenzyl)piperidine-2,6-dione (500 mg, 333 μmol) inTFA(1.5 mL) and TfOH (0.5 mL) was degassed and purged with N₂ three times. Then the mixture was stirred at 60° C. for 1 hr under N₂ atmosphere. Upon completion, the reaction mixture was concentrated under reduced pressure to remove solvent. The crude residue was purified by reversed-phase HPLC (0.1% FA condition) to give the title compound (150 mg, 62% yield) as a white solid. LC-MS (ESI⁺) m/z 480.3 (M+H)⁺.

### (1R,5R)-3-(4-chloro-2-fluorophenyl)-3,6-diazabicyclo[3.2.0]heptane (Intermediate SM)

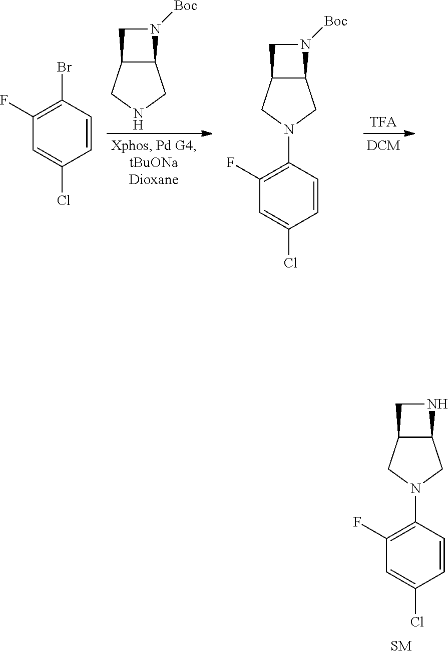

### Step 1—Tert-butyl (1S,5R)-3-(4-chloro-2-fluorophenyl)-3,6-diazabicyclo[3.2.0]heptane-6-carboxylate

To a solution of tert-butyl (1S,5R)-3,6-diazabicyclo[3.2.0]heptane-6-carboxylate (946 mg, 4.77 mmol, CAS #370882-66-9) and 1-bromo-4-chloro-2-fluorobenzene (1.5 g, 7.2 mmol, CAS #1996-29-8) in toluene (1 mL) was added Xphos Pd G4 (410 mg, 477 μmo) and t-BuONa (1.38 g, 14.3 mmol) at 25° C. under nitrogen atmosphere. Then the mixture was stirred at 100° C. for 1 hr under nitrogen atmosphere. Upon completion, the reaction mixture was concentrated in vacuo to give the crude residue. The residue was purified by column chromatography (SiO₂, Petroleum ether/Ethyl acetate=5/1 to 3/1) to give the title compound (1.1 g. 71% yield) as a white solid. LC-MS (ESI⁺) m/z 271.1 (M−56)⁺; ¹H NMR (400 MHz, DMSO-d6) δ=7.32 (br d, J=13.2 Hz, 1H), 7.17-7.11 (m, 1H), 6.97-6.89 (m, 1H), 4.66 (dd, J=4.4, 6.4 Hz, 1H), 3.98-3.86 (m, 2H), 3.63-3.44 (m, 2H), 3.10 (br s, 1H), 2.86 (dd, J=6.4, 10.4 Hz, 1H), 2.74 (dd, J=4.0, 11.2 Hz, 1H), 1.38 (br s, 9H).

### Step 2—(1R,5R)-3-(4-chloro-2-fluorophenyl)-3,6-diazabicyclo[3.2.0]heptane

To a solution of tert-butyl (1S,5R)-3-(4-chloro-2-fluorophenyl)-3,6-diazabicyclo[3.2.0]heptane-6-carboxylate (1.1 g, 3.37 mmol) in anhydrous DCM (10 mL) was added TFA (4.22 g, 37.0 mmol, 2.75 mL) at 25° C., then the reaction was stirred at 25° C. for 1 hr. Upon completion, the reaction mixture was concentrated in vacuo to give the crude residue. The residue was purified by reversed-phase HPLC (0.8 g/L ammonium bicarbonate) to give the title compound (700 mg. 82% yield) as a white solid. LC-MS (ESI⁺) m/z 209.9 (M−56)⁺; ¹H NMR (400 MHz. DMSO-d₆) δ=7.39-7.28 (m, 1H), 7.19-7.11 (m, 1H), 7.00-6.91 (m, 1H), 4.69-4.64 (m, 1H), 3.98-3.80 (m, 3H), 3.76-3.60 (m, 2H), 3.01-2.81 (m, 3H).

### 3-(3-Fluoro-4-((1S,5R)-3-(2-fluoro-4-(4,4,5,5-tetramethyl-1,3,2-dioxaborolan-2-yl)phenyl)-3,6-diazabicyclo[3.2.0]heptan-6-yl)phenyl)piperidine-2,6-dione (Intermediate SN)

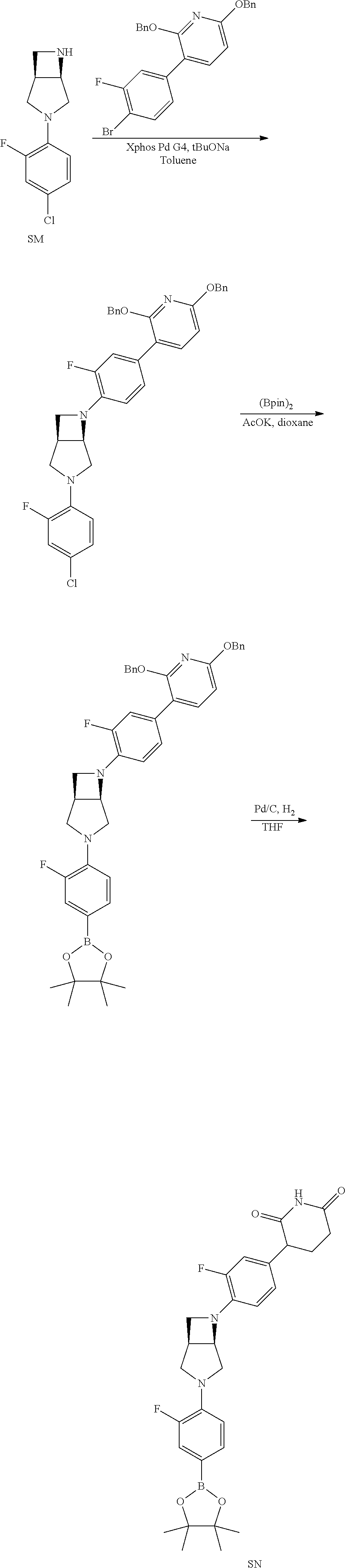

### Step 1—(1S,5R)-6-(4-(2,6-bis(benzyloxy)pyridin-3-yl)-2-fluorophenyl)-3-(4-chloro-2-fluorophenyl)-3,6-diazabicyclo[3.2.0]heptane

To a solution of 2,6-bis(benzyloxy)-3-(4-bromo-3-fluorophenyl)pyridine (891 mg, 1.92 mmol. CAS #2902652-28-0) and (1R,5R)-3-(4-chloro-2-fluorophenyl)-3,6-diazabicyclo[3.2.0]heptane (870 mg, 3.84 mmol, Intermediate SM) in toluene (5 mL) was added t-BuONa (553 mg, 5.76 mmol) and Xphos Pd G4 (165 mg, 191 μmol) at 25° C. under nitrogen atmosphere. Then the mixture was stirred at 100° C. for 1 hr under nitrogen atmosphere. Upon completion, the reaction mixture was concentrated in vacuo to get the crude residue. The residue was purified by column chromatography (SiO₂, Petroleum ether/Ethyl acetate=1/0 to 10/1) give the title compound (1 g, 73% yield) as a yellow solid. LC-MS (ESI⁺) m/z 610.2 (M+H)⁺.

### Step 2—(1S,5R)-6-(4-(2,6-bis(benzyloxy)pyridin-3-yl)-2-fluorophenyl)-3-(2-fluoro-4-(4,4,5,5-tetramethyl-1,3,2-dioxaborolan-2-yl)phenyl)-3,6-diazabicyclo[3.2.0]heptane

To a solution of 4,4,5,5-tetramethyl-2-(4,4,5,5-tetramethyl-1,3,2-dioxaborolan-2-yl)-1,3,2-dioxaborolane (199 mg, 784 μmol) and (1S,5R)-6-(4-(2,6-bis(benzyloxy)pyridin-3-yl)-2-fluorophenyl)-3-(4-chloro-2-fluorophenyl)-3,6-diazabicyclo[3.2.0]heptane (570 mg, 523 μmol) in dioxane (3 mL) was added XPhos Pd G3 (44.2 mg, 52.3 μmol) and AcOK (154 mg, 1.57 mmol) at 25° C. under nitrogen atmosphere. Then the mixture was stirred at 80° C. for 2 hrs under nitrogen atmosphere. Upon completion, the reaction mixture was filtered and the filtrate was concentrated in vacuo to give the crude residue. The crude residue was purified by column chromatography (SiO₂, Petroleum ether/Ethyl acetate=8/1 to 4/1) to give the title compound (300 mg. 73% yield) as a yellow solid. LC-MS (ESI⁺) nm/z 702.2 (M+H)⁺.

### Step 3—3-(3-Fluoro-4-((1S,5R)-3-(2-fluoro-4-(4,4,5,5-tetramethyl-1,3,2-dioxaborolan-2-yl)phenyl)-3,6-diazabicyclo[3.2.0]heptan-6-yl)phenyl)piperidine-2,6-dione

To a solution of (1S,5R)-6-(4-(2,6-bis(benzyloxy)pyridin-3-yl)-2-fluorophenyl)-3-(2-fluoro-4-(4,4,5,5-tetramethyl-1,3,2-dioxaborolan-2-yl)phenyl)-3,6-diazabicyclo[3.2.0]heptane (300 mg, 427 μmol) in anhydrous THF (2 mL) was added Pd/C (3.00 g, 2.82 mmol, 10 wt %) at 30° C. under nitrogen atmosphere. Then the reaction was stirred at 30° C. for 12 hrs under H₂ atmosphere (40 psi). Upon completion, the reaction was filtered through kieselguhr very carefully, and the filtrated was concentrated in vacuo to give the title compound (170 mg) as a yellow solid. LC-MS (ESI⁺) m/z 524.2 (M+H)⁺.

### 3-(4-(4-(4-Chlorobenzyl)piperidin-1-yl)-3-fluorophenyl)piperidine-2,6-dione (Intermediate SO)

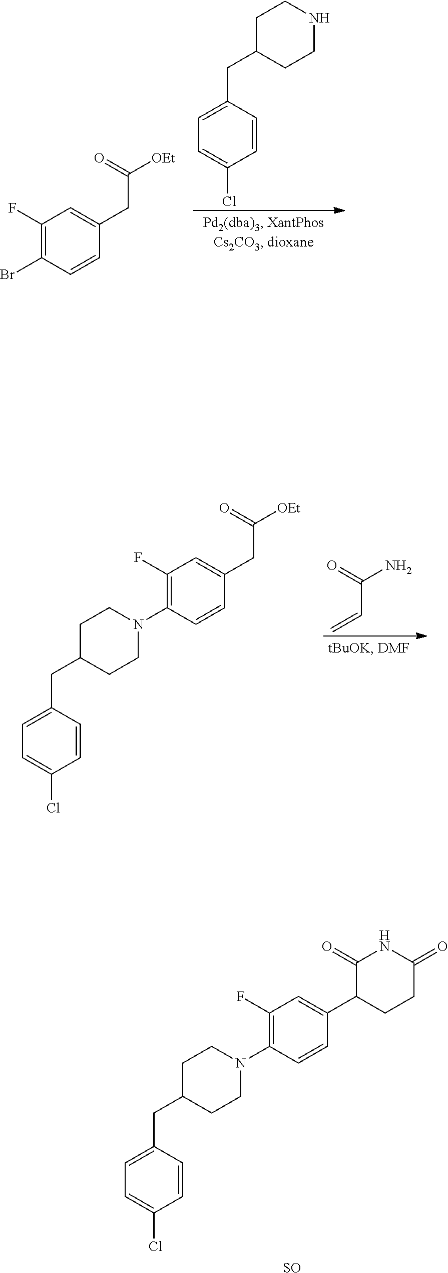

### Step 1—Ethyl 2-(4-(4-(4-chlorobenzyl)piperidin-1-yl)-3-fluorophenyl)acetate

To a solution of ethyl 2-(4-bromo-3-fluorophenyl)acetate (5.60 g. 21.4 mmol, CAS #1296223-82-9) in dioxane (40 mL) was added 4-(4-chlorobenzyl)piperidine (3 g, 14 mmol, CAS #36938-76-8), Cs₂CO₃ (13.9 g, 42.9 mmol), Xantphos (827 mg, 1.43 mmol) and Pd₂(dba)₃ (654 mg, 715 μmol). The reaction was then stirred at 100° C. for 12 hrs under N₂ atmosphere. Upon completion, the reaction mixture was concentrated in vacuo to give the crude residue. The crude residue was purified by reversed-phase HPLC (0.8 g/L ammonium bicarbonate) to give the title compound (1.2 g, 10% yield) as a brown oil. LC-MS (ESI⁺) m/z 390.2 (M+H)⁺.

### Step 2—3-(4-(4-(4-Chlorobenzyl)piperidin-1-yl)-3-fluorophenyl)piperidine-2,6-dione

To a solution of ethyl 2-(4-(4-(4-chlorobenzyl)piperidin-1-yl)-3-fluorophenyl)acetate (600 mg, 1.54 mmol) in DMF (6 mL) was added acrylamide (109 mg, 1.54 mmol, 106 μL) and KOtBu (1 M, 1.54 mL) at 0° C. The reaction was then stirred at 0° C. for 0.5 hrs. Upon completion, the reaction mixture was quenched with HCl (2 mL) and extracted with ethyl acetate (3×10 mL). The combined organic layers were washed with brine (40 mL) and dried over anhydrous sodium sulfate, filtered and the filtrate was concentrated in vacuo to give the crude residue. The residue was purified by column chromatography (SiO₂, Petroleum ether:Ethyl acetate=10/1 to 1/1) to give the title compound (300 mg, 32% yield) as a brown oil. LC-MS (ESI⁺) m/z 415.1 (M+H)⁺.

### 3-(3-fluoro-4-(4-(4-(4,4,5,5-tetramethyl-1,3,2-dioxaborolan-2-yl)benzyl)piperidin-1-yl)phenyl)piperidine-2,6-dione (Intermediate SP)

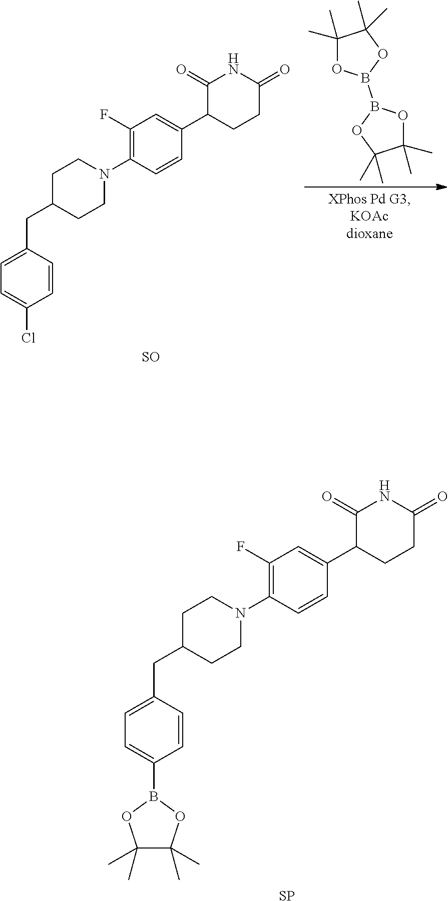

To a solution of 3-(4-(4-(4-chlorobenzyl)piperidin-1-yl)-3-fluorophenyl)piperidine-2,6-dione (250 mg, 602 μmol, Intermediate SO) in dioxane (5 mL) was added 4,4,4′,4′,5,5,5′,5′-octamethyl-2,2′-bi(1,3,2-dioxaborolane) (306 mg, 1.21 mmol), AcOK (177 mg, 1.81 mmol) and XPhos Pd G3 (51.0 mg, 60.2 μmol) and the reaction was stirred at 80° C. for 2 hrs. On completion, the reaction mixture was concentrated in vacuo to give the crude residue. The residue was purified by column chromatography (SiO₂, Dichloromethane:Methanol=50/1 to 20/1) to give the title compound (250 mg, 59% yield) as a brown solid. LC-MS (ESI⁺) m/z 507.2 (M+H)⁺.

### 5-Bromo-2-(4-(4-chlorophenyl)piperidin-1-yl)-3-fluoropyridine (Intermediate SQ)

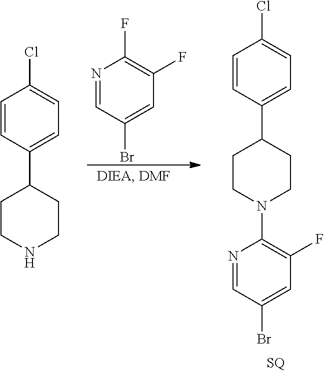

To a solution of 5-bromo-2,3-difluoro-pyridine (12.5 g, 64 mmol) in DMF (150 mL) was added TEA (19.6 g, 193 mmol, 26.9 mL) and 4-(4-chlorophenyl)piperidine (12.6 g, 64.4 mmol). The mixture was then stirred at 80° C. for 12 hrs. Upon completion, the mixture was quenched with H₂O (500 mL), then filtered to give a residue to give the title compound (20.4 g) as a white solid. LC-MS (ESI⁺) m/z 370.9 (M+H)⁺: ¹H NMR (400 MHz, DMSO-d₆) δ=8.13 (s, 1H), 7.87 (d, J=12.8 Hz, 1H), 7.42-7.24 (m, 4H), 4.09 (d, J=11.2 Hz, 2H), 2.97 (s, 2H), 2.79 (d, J=11.6 Hz, 1H), 1.90-1.63 (m, 4H).

### 3-(5-Fluoro-6-(4-(4-(4,4,5,5-tetramethyl-1,3,2-dioxaborolan-2-yl)phenyl)piperidin-1-yl)pyridin-3-yl)piperidine-2,6-dione (Intermediate SR)

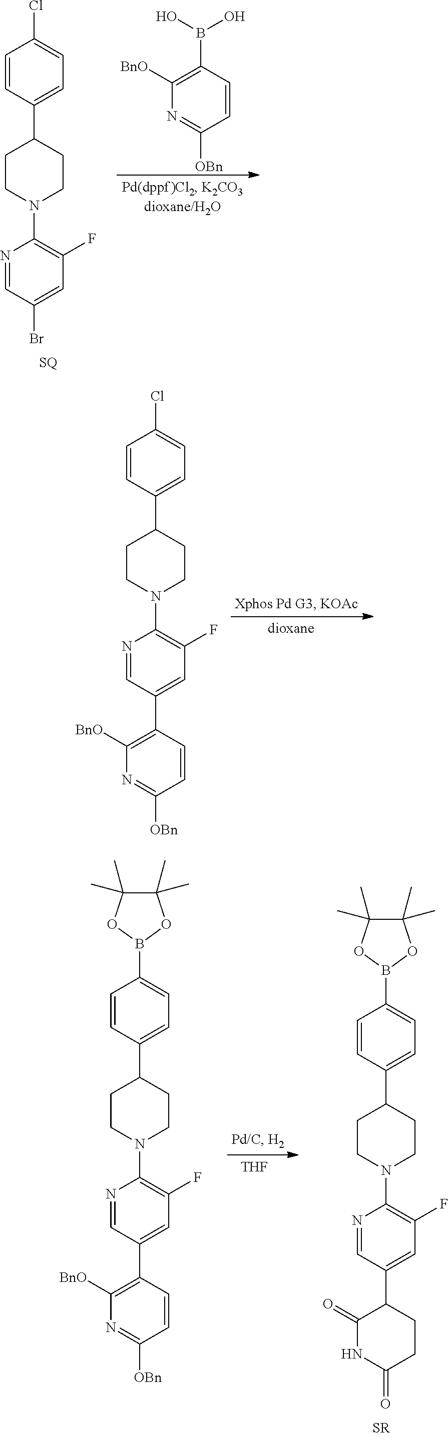

### Step 1—2,6-Bis(benzyloxy)-6′-(4-(4-chlorophenyl)piperidin-1-yl)-5′-fluoro-3,3′-bipyridine

A mixture of 2,6-dibenzyloxy-3-(4,4,5,5-tetramethyl-1,3,2-dioxaborolan-2-yl)pyridine (7.34 g, 17.6 mmol, synthesized via Step 1 of Intermediate AT), 5-bromo-2-[4-(4-chlorophenyl)-1-piperidyl]-3-fluoro-pyridine (5 g, 10 mmol, Intermediate SQ), Pd(dppf)Cl₂·CH₂Cl₂ (1.10 g, 1.35 mmol), and K₂CO₃ (5.61 g, 40.6 mmol) in dioxane (50 mL) H₂O (10 mL) was degassed and purged with N₂ three times. Then the mixture was stirred at 100° C. for 12 hrs under N₂ atmosphere. Upon completion, the mixture was concentrated under reduced pressure to give a residue. The mixture was purified by MPLC (SiO2, PE:EA=50: I to 30:1) to give the title compound (7 g, 83% yield) as a white solid. LC-MS (ESI⁺) m/z 580.2 (M+H)⁺.

### Step 2—Benzyl 2,6-bis(benzyloxy)-5′-fluoro-6′-(4-(4-(4,4,5,5-tetramethyl-1,3,2-dioxaborolan-2-yl)phenyl)piperidin-1-yl)-3,3′-bipyridine

A mixture of 2,6-dibenzyloxy-3-[6-[4-(4-chlorophenyl)-1-piperidyl]-5-fluoro-3-pyridyl]pyridine (4 g, 6.90 mmol), XPhos Pd G3 (584 mg, 689 μmol), KOAc (677 mg, 6.90 mmol) and 4,4,5,5-tetramethyl-2-(4,4,5,5-tetramethyl-1,3,2-dioxaborolan-2-yl)-1,3,2-dioxaborolane (3.50 g, 13.8 mmol) in dioxane (50 mL) was degassed and purged with N₂ three times. Then the mixture was stirred at 80° C. for 12 hrs under N₂ atmosphere. Upon completion, the mixture was concentrated under reduce pressure to give a residue. The mixture was purified by MPLC (SiO₂, PE; EA=50:1 to 30:1) to give the title compound (5 g, 90% yield) as a white solid. LC-MS (ESI⁺) m/z 672.3 (M+H)⁺: ¹H NMR (400 MHz. DMSO-d6) δ=8.16 (s, 1H), 7.74 (d, J=8.0 Hz, 2H), 7.60-7.47 (m, 2H), 7.43-7.29 (m, 9H), 7.26-7.22 (m, 3H), 6.44 (d, J=8.0 Hz, 1H), 5.32 (s, 2H), 5.26 (s, 2H), 4.19 (d, J=12.8 Hz, 2H), 2.96 (d, J=1.6 Hz, 2H), 2.73 (s, 1H), 1.90 (dd, J=3.2, 7.2 Hz, 4H), 1.30 (s, 12H).

### Step 3—3-(5-Fluoro-6-(4-(4-(4,4,5,5-tetramethyl-1,3,2-dioxaborolan-2-yl)phenyl)piperidin-1-yl)pyridin-3-yl)piperidine-2,6-dione

A mixture of 2,6-dibenzyloxy-3-[5-fluoro-6-[4-[4-(4,4,5,5-tetramethyl-1,3,2-dioxaborolan-2-yl)phenyl]-1-piperidyl]-3-pyridyl]pyridine (5 g, 7.44 mmol), Pd/C (25 g, 23.5 mmol, 2.35 mL, 10 wt %), and Pd(OH)₂ (12.5 g, 17.8 mmol) in THF (50 mL) was degassed and purged with N₂ three times. Then the mixture was stirred at 40° C. for 12 hrs under H₂ atmosphere. Upon completion, the mixture was filtered to recycle the Pd/C then concentrated under reduce pressure to give a residue to give the title compound (3.2 g) as a yellow solid. LC-MS (ESI⁺) m/z 494.2 (M+H)⁺.

### Tert-butyl 4-(4-chloro-2-fluoro-phenyl)piperidine-1-carboxylate (Intermediate SS)

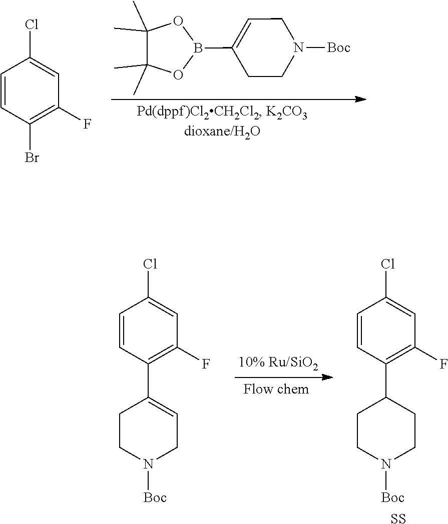

### Step 1—Tert-butyl 4-(4-chloro-2-fluoro-phenyl)-3,6-dihydro-2H-pyridine-1-carboxylate

To a solution of 1-bromo-4-chloro-2-fluoro-benzene (10 g, 47.6 mmol, CAS #1996-29-8) and tert-butyl 4-(4,4,5,5-tetramethyl-1,3,2-dioxaborolan-2-yl)-3,6-dihydro-2H-pyridine-1-carboxylate (14.8 g, 47.6 mmol, CAS #286961-14-6) in dioxane (300 mL) and H₂O (50 mL) was added K₂CO₃ (19.8 g, 143 mmol) and Pd(dppf)Cl₂·CH₂Cl₂ (3.90 g, 4.77 mmol). Then the mixture was degassed under vacuum, purged with N₂ three times and the mixture was stirred at 80° C. for 2 hrs under N₂ atmosphere. Upon completion, the reaction mixture filtered with celite and was quenched with water (200 mL). Then the mixture extracted with DCM (100 mL×3). The combined organic layers were washed with saturated NaCl (200 mL), dried over Na₂SO₄, filtered and concentrated in vacuo to give a residue. The residue was purified by column chromatography (SiO₂, PE/EA=1/0 to 10/1) to give the title compound (4.2 g, 28% yield) as white solid. LC-MS (ESI⁺) m/z 256.0 (M−56)⁺.

### Step 2—Tert-butyl 4-(4-chloro-2-fluoro-phenyl)piperidine-1-carboxylate

Tert-butyl 4-(4-chloro-2-fluoro-phenyl)-3,6-dihydro-2H-pyridine-1-carboxylate (4 g) in THF (80 mL) was ran through a flow reactor with a fixed bed of granular catalyst 10% Ru/SiO₂. The H₂ back pressure regulator was adjusted to 1.5 MPa, and the flow rate of H₂ was 30 mL/min. Then the solution was pumped at 0.303 mL/min through the fixed bed at 60° C. Upon completion, the reaction mixture was quenched with water (200 mL) and diluted with EA (100 mL×3). The combined organic layers were washed with saturated NaCl (200 mL), dried over Na₂SO₄, filtered and concentrated in vacuo to give a residue. The residue was purified by column chromatography (SiO₂, DCM/MeOH=100/1 to 70/1) to give the title compound (3.3 g. 82% yield) as colorless oil. LC-MS (ESI⁺) m/z 257.9 (M−56)⁺.

### 7-Fluoro-4-[3-fluoro-4-(4-piperidyl)phenyl]-N,N-dimethyl-6-[1-[3-(triazol-1-yl)propanoyl]-3,6-dihydro-2H-pyridin-5-yl]-1H-indole-2-carboxamide (Intermediate ST)

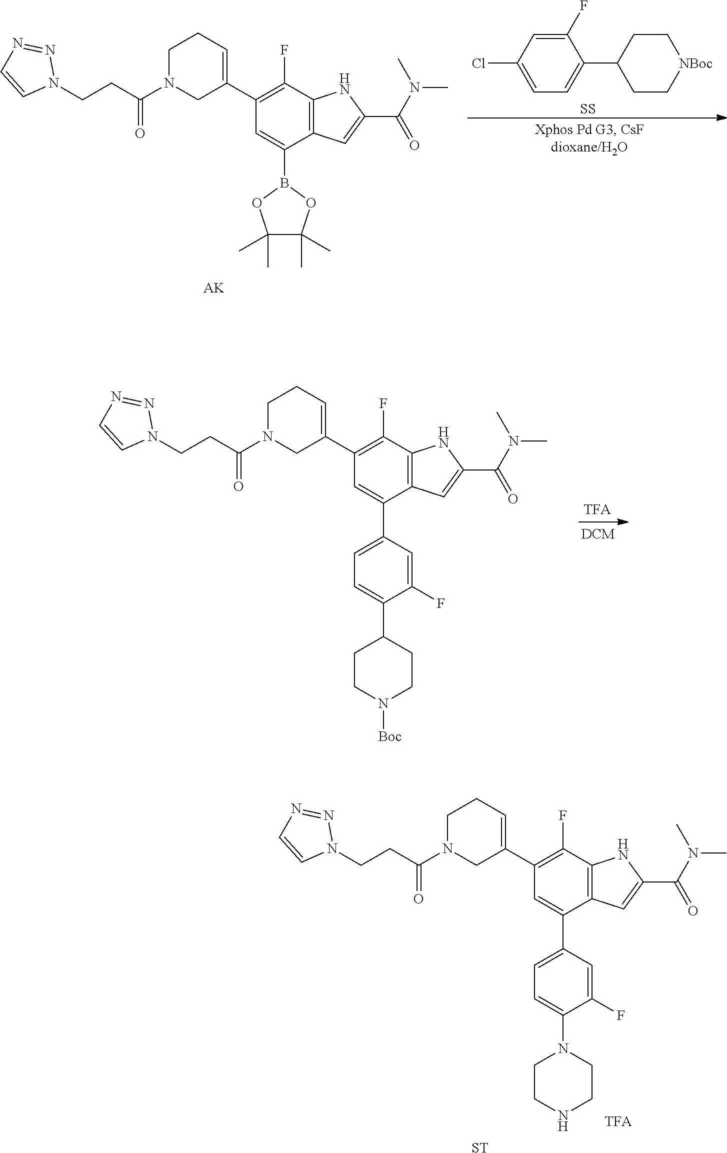

### Step 1—Tert-butyl 4-[4-[2-(dimethylcarbamoyl)-7-fluoro-6-[1-[3-(triazol-1-yl)propanoyl]-3,6-dihydro-2H-pyridin-5-yl]-1H-indol-4-yl]-2-fluoro-phenyl]piperidine-1-carboxylate

To a solution of 7-fluoro-N,N-dimethyl-4-(4,4,5,5-tetramethyl-1,3,2-dioxaborolan-2-yl)-6-[1-[3-(triazol-1-yl)propanoyl]-3,6-dihydro-2H-pyridin-5-yl]-1H-indole-2-carboxamide (250 mg, 466 μmol, Intermediate AK) and tert-butyl 4-(4-chloro-2-fluoro-phenyl)piperidine-1-carboxylate (146 mg, 466 μmol, Intermediate SS) in dioxane:H₂O=5:1 (6 mL) was added XPhos Pd G3 (39.5 mg, 46.6 μmol) and CsF (212.39 mg, 1.40 mmol) and at 25° C. under nitrogen atmosphere. The mixture was then stirred at 80° C. for 2 hrs under nitrogen atmosphere. Upon completion, the reaction mixture filtered with diatomaceous and was quenched with water (20 mL), then the mixture was extracted with DCM (10 mL×3). The combined organic layers were washed with saturated NaCl (20 mL), dried over Na₂SO₄, filtered and concentrated in vacuo to give a residue. The residue was purified by column chromatography (SiO₂, PE/EA=10/1 to 4/1) and was triturated with ACN (2 mL) to give the title compound (157 mg, 48% yield) as white solid. ¹H NMR (400 MHz, DMSO-d₆) δ 12.23 (s, 1H), 8.20-8.00 (m, 1H), 7.75-7.62 (m, 1H), 7.53-7.39 (m, 3H), 7.17-7.01 (m, 1H), 6.87 (s, 1H), 6.17 (s, 1H), 4.68-4.57 (m, 2H), 4.41-4.28 (m, 2H), 4.19-4.03 (m, 2H), 3.67-3.58 (m, 2H), 3.14-3.03 (m, 6H), 2.93-2.79 (m, 2H), 2.54 (s, 3H), 2.36-2.24 (m, 2H), 1.83-1.72 (m, 2H), 1.65-1.55 (m, 2H), 1.43 (s, 9H). LC-MS (ESI⁺) m/z 688.3 (M+H)⁺.

### Step 2—7-Fluoro-4-[3-fluoro-4-(4-piperidyl)phenyl]-N,N-dimethyl-6-[1-[3-(triazol-1-yl)propanoyl]-3,6-dihydro-2H-pyridin-5-yl]-1H-indole-2-carboxamide

To a solution of tert-butyl 4-[4-[2-(dimethylcarbamoyl)-7-fluoro-6-[1-[3-(triazol-1-yl)propanoyl]-3,6-dihydro-2H-pyridin-5-yl]-1H-indol-4-yl]-2-fluoro-phenyl]piperidine-1-carboxylate (150 mg, 218 μmol) in DCM (2 mL) was added TFA (1.54 g, 13.5 mmol) at 25° C., then the mixture was stirred at 25° C. for 0.5 hr. Upon completion, the reaction mixture was concentrated in vacuo to give the title compound (151 mg. TFA) as black brown liquid. LC-MS (ESI⁺) m/z 588.4 (M+H)⁺.

### 7-Fluoro-N,N-dimethyl-4-[2-methyl-4-(4-piperidyl)phenyl]-6-[1-[3-(triazol-1-yl)propanoyl]-3,6-dihydro-2H-pyridin-5-yl]-1H-indole-2-carboxamide (Intermediate SU)

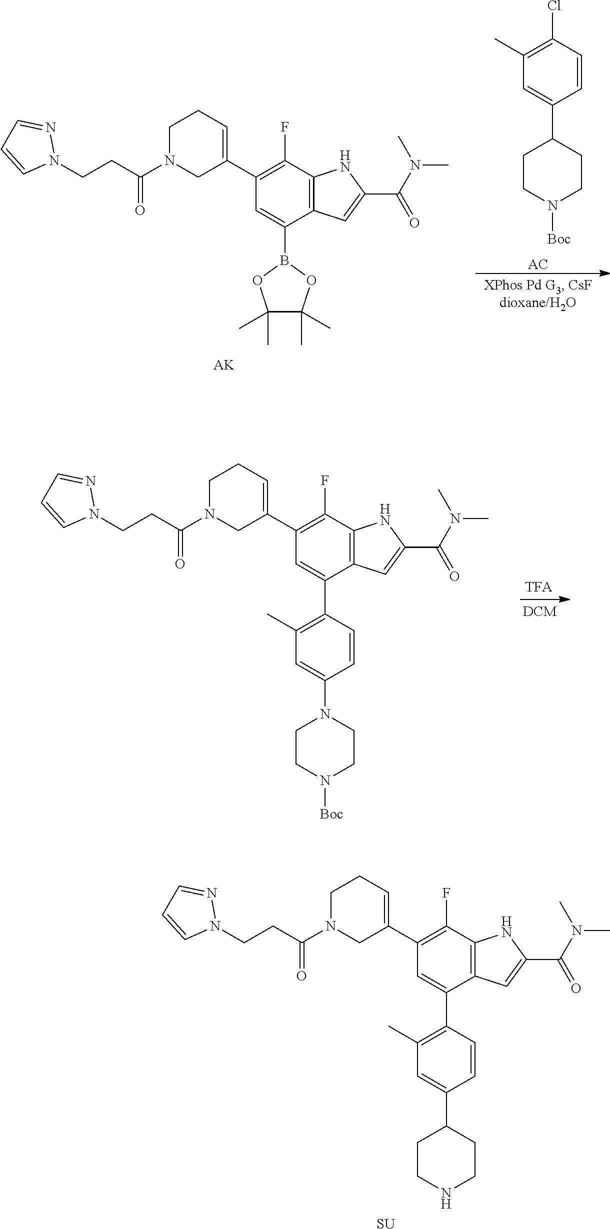

### Step 1—Tert-butyl 4-[4-[2-(dimethylcarbamoyl)-7-fluoro-6-[1-[3-(triazol-1-yl)propanoyl]-3,6-dihydro-2H-pyridin-5-yl]-1H-indol-4-yl]-3-methyl-phenyl]piperidine-1-carboxylate

A mixture of 7-fluoro-N,N-dimethyl-4-(4,4,5,5-tetramethyl-1,3,2-dioxaborolan-2-yl)-6-[1-[3-(triazol-1-yl)propanoyl]-3,6-dihydro-2H-pyridin-5-yl]-1H-indole-2-carboxamide (220 mg, 410 μmol, Intermediate AK), tert-butyl 4-(4-chloro-3-methyl-phenyl)piperidine-1-carboxylate (152 mg, 492 μmol, Intermediate AC), XPhos Pd G3 (34.7 mg, 41.0 μmol) and CsF (186 mg, 1.23 mmol) in dioxane (3 mL) and H₂O (0.5 mL) was degassed and purged with N₂ three times. Then the mixture was stirred at 80° C. for 2 hrs under N₂ atmosphere. Upon completion, the reaction mixture was poured into H₂O (20 mL), and then extracted with EA (20 mL×3). The combined organic layers were washed with brine (20 mL), dried over Na₂SO₄ and filtered. The filtrate was concentrated in vacuo to give a residue. The residue was purified by column chromatography (SiO₂, EA: THF=20/1 to 5/1) to give the title compound (200 mg, 64% yield) as a gray solid. ¹H NMR (400 MHz, DMSO-d₆) δ 12.22-12.02 (m, 1H), 8.17-8.00 (m, 1H), 7.75-7.60 (m, 1H), 7.21 (d, J=7.2 Hz, 2H), 7.16-7.09 (m, 1H), 6.91-6.84 (m, 1H), 6.83-6.60 (m, 1H), 6.36 (s, 1H), 6.20-6.08 (m, 1H), 5.75 (s, 1H), 4.61 (d, J=7.2 Hz, 2H), 4.39-4.27 (m, 2H), 4.16-4.01 (m, 2H), 3.66-3.57 (m, 2H), 3.14-3.03 (m, 6H), 2.74-2.67 (m, 1H), 2.33 (d, J=1.6 Hz, 2H), 2.13 (s, 2H), 1.81 (d, J=12.0 Hz, 2H), 1.60-1.51 (m, 2H), 1.45-1.39 (m, 9H), 1.35 (s, 3H): LC-MS (ESI⁺) m/z 684.3 (M+H)⁺.

### Step 2—7-Fluoro-N,N-dimethyl-4-[2-methyl-4-(4-piperidyl)phenyl]-6-[1-[3-(triazol-1-yl)propanoyl]-3,6-dihydro-2H-pyridin-5-yl]-1H-indole-2-carboxamide

To a solution of tert-butyl 4-[4-[2-(dimethylcarbamoyl)-7-fluoro-6-[1-[3-(triazol-1-yl)propanoyl]-3,6-dihydro-2H-pyridin-5-yl]-1H-indol-4-yl]-3-methyl-phenyl]piperidine-1-carboxylate (100 mg, 146 μmol) in DCM (1 mL) and TFA (0.2 mL) was stirred at 25° C. for 1 hr. Upon completion, the reaction mixture was concentrated in vacuo to give the title compound (95.0 mg, 93% yield, TFA) as a brown oil. LC-MS (ESI⁺) m/z 584.4 (M+H)⁺.

### 7-Fluoro-N,N-dimethyl-4-[2-methyl-4-(4-piperidyl)phenyl]-6-[1-(3-pyrazol-1-ylpropanoyl)-3,6-dihydro-2H-pyridin-5-yl]-1H-indole-2-carboxamide (Intermediate SV)

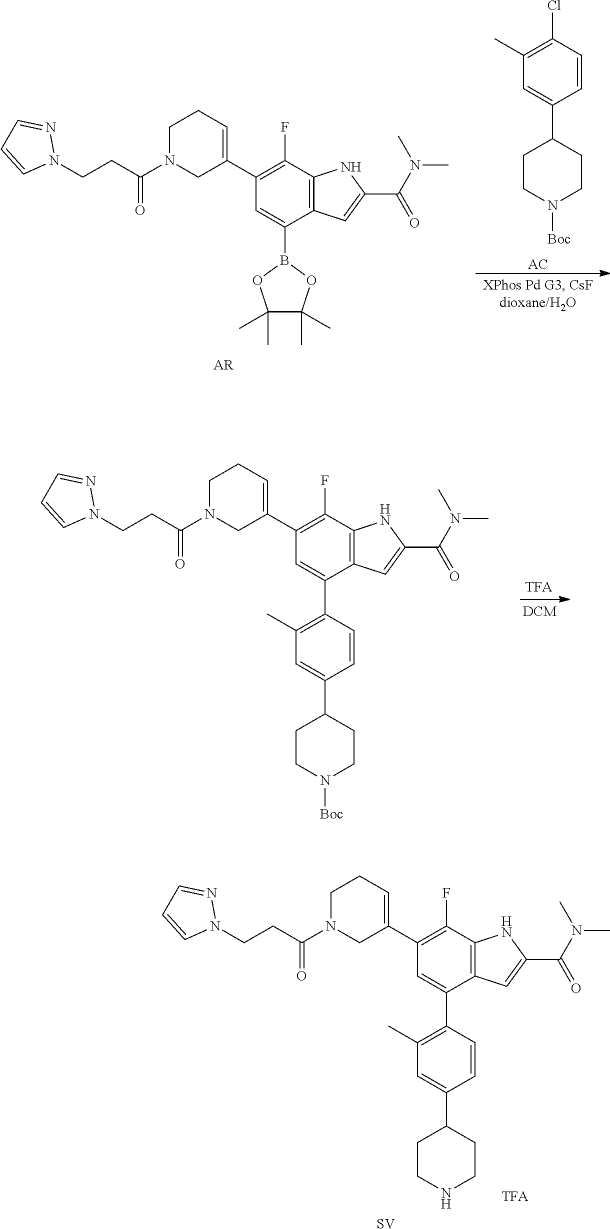

### Step 1—Tert-butyl 4-[4-[2-(dimethylcarbamoyl)-7-fluoro-6-[1-(3-pyrazol-1-ylpropanoyl)-3,6-dihydro-2H-pyridin-5-yl]-1H-indol-4-yl]-3-methyl-phenyl]piperidine-1-carboxylate

A mixture of tert-butyl 4-(4-chloro-3-methyl-phenyl)piperidine-1-carboxylate (376 mg, 1.21 mmol. Intermediate AC). 7-fluoro-N,N-dimethyl-6-[1-(3-pyrazol-1-ylpropanoyl)-3,6-dihydro-2H-pyridin-5-yl]-4-(4,4,5,5-tetramethyl-1,3,2-dioxaborolan-2-yl)-1H-indole-2-carboxamide (500 mg, 933 μmol, Intermediate AR), XPhos Pd G3 (79.0 mg, 93.3 μmol), and CsF (425 mg, 2.80 mmol) in dioxane (5 mL) and H₂O (1 mL) was degassed and purged with N₂ three times. Then the mixture was stirred at 80° C. for 6 hr under N₂ atmosphere. Upon completion, the reaction mixture was concentrated in vacuo to give the residue. The residue was purified by column chromatography (SiO₂, EA:THF=from 1:0 to 10:1) to give the title compound (360 mg, 56% yield) as yellow solid. ¹H NMR (400 MHz, DMSO-d₆) δ 12.16-12.09 (m, 1H), 7.76-7.66 (m, 1H), 7.44-7.39 (m, 1H), 7.21 (s, 1H), 7.16-7.11 (m, 1H), 6.89-6.78 (m, 1H), 6.36 (d, J=2.0 Hz, 1H), 6.22-6.11 (m, 2H), 4.39-4.32 (m, 4H), 4.10 (d, J=10.8 Hz, 2H), 3.66-3.60 (m, 1H), 3.58-3.55 (m, 1H), 3.16-3.03 (m, 6H), 2.99-2.93 (m, 3H), 2.87-2.81 (m, 1H), 2.75-2.67 (m, 1H), 2.37-2.23 (m, 3H), 2.13 (s, 3H), 1.81 (d, J=13.2 Hz, 2H), 1.59-1.48 (m, 2H), 1.44-1.41 (m, 9H); LC-MS (ESI⁺) m/z 683.2 (M+H)⁺.

### Step 2—7-Fluoro-N,N-dimethyl-4-[2-methyl-4-(4-piperidyl)phenyl]-6-[1-(3-pyrazol-1-ylpropanoyl)-3,6-dihydro-2H-pyridin-5-yl]-1H-indole-2-carboxamide

To a solution of tert-butyl 4-[4-[2-(dimethylcarbamoyl)-7-fluoro-6-[1-(3-pyrazol-1-ylpropanoyl)-3,6-dihydro-2H-pyridin-5-yl]-1H-indol-4-yl]-3-methyl-phenyl]piperidine-1-carboxylate (140 mg, 205 μmol) in DCM (2 mL) was added TFA (307 mg, 2.69 mmol). Then the mixture was stirred at 25° C. for 1 hr. Upon completion the reaction mixture was concentrated in vacuo to give the title compound (140 mg, 98% yield, TFA) as yellow oil. LC-MS (ESI⁺) m/z 583.2 (M+H)⁺.

### 7-Fluoro-4-[3-fluoro-4-(4-piperidyl)phenyl]-N,N-dimethyl-6-[1-(3-pyrazol-1-ylpropanoyl)-3,6-dihydro-2H-pyridin-5-yl]-1H-indole-2-carboxamide (Intermediate SW)

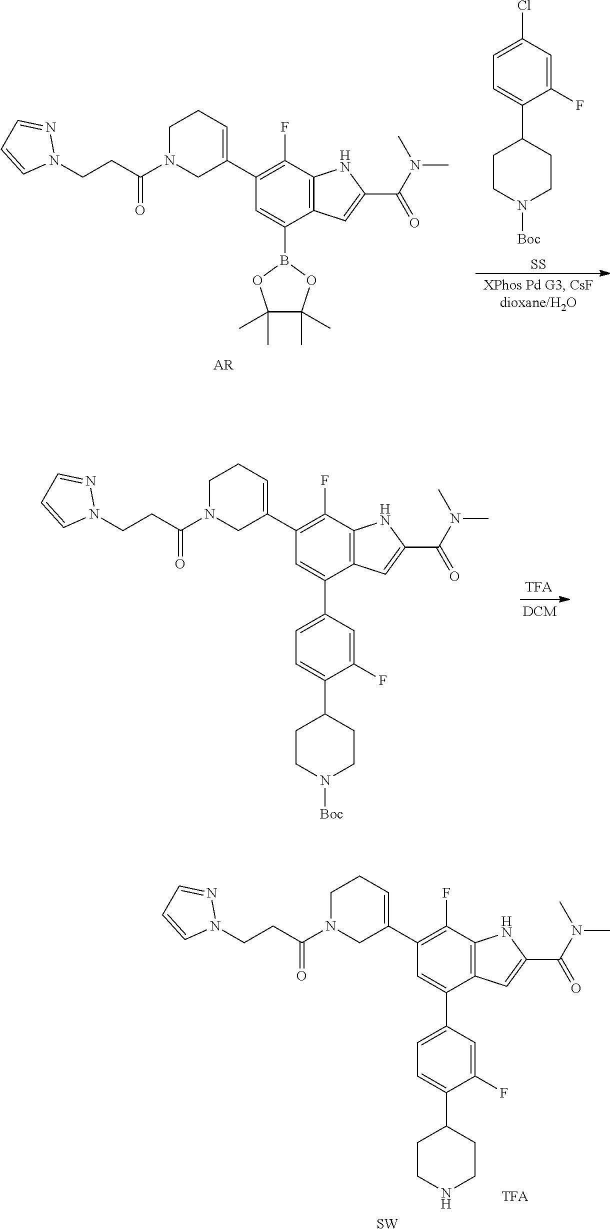

### Step 1—Tert-butyl 4-[4-[2-(dimethylcarbamoyl)-7-fluoro-6-[1-(3-pyrazol-1-ylpropanoyl)-3,6-dihydro-2H-pyridin-5-yl]-1H-indol-4-yl]-2-fluoro-phenyl]piperidine-1-carboxylate

A mixture of 7-fluoro-N,N-dimethyl-6-[1-(3-pyrazol-1-ylpropanoyl)-3,6-dihydro-2H-pyridin-5-yl]-4-(4,4,5,5-tetramethyl-1,3,2-dioxaborolan-2-yl)-1H-indole-2-carboxamide (500 mg, 933 μmol, Intermediate AR), CsF (425 mg, 2.80 mmol), tert-butyl 4-(4-chloro-2-fluoro-phenyl)piperidine-1-carboxylate (293 mg, 933 μmol, Intermediate SS), and XPhos Pd G₃ (79.0 mg, 93.3 μmol) in dioxane (8 mL) and H₂O (1.5 mL) was degassed and purged with N₂ three times. Then the mixture was stirred at 80° C. for 6 hr under N₂ atmosphere. Upon completion, the reaction mixture was extracted with EA (30 mL×3), and the combined layer was washed with brine, dried over Na₂SO₄, and concentrated in vacuo to give the residue. The residue was triturated by EA (30 mL) to give the title compound (500 mg, 80% yield). ¹H NMR (400 MHz, DMSO-d₆) δ 12.22 (d, J=7.2 Hz, 1H), 7.76-7.67 (m, 1H), 7.51-7.35 (m, 4H), 7.12-7.02 (m, 1H), 6.87 (s, 1H), 6.24-6.12 (in. 2H), 4.41-4.28 (m, 4H), 4.10 (d, J=11.2 Hz, 1H), 3.67-3.54 (m, 2H), 3.21-2.96 (m, 10H), 2.92-2.82 (m, 2H), 2.36-2.25 (m, 2H), 1.78 (d, J=12.4 Hz, 2H), 1.66-1.54 (m, 2H), 1.43 (s, 9H): LC-MS (ESI⁺) m/z 687.1 (M+H)⁺.

### Step 2—7-Fluoro-4-[3-fluoro-4-(4-piperidyl)phenyl]-N,N-dimethyl-6-[1-(3-pyrazol-1-ylpropanoyl)-3,6-dihydro-2H-pyridin-5-yl]-1H-indole-2-carboxamide

To a solution of tert-butyl 4-[4-[2-(dimethylcarbamoyl)-7-fluoro-6-[1-(3-pyrazol-1-ylpropanoyl)-3,6-dihydro-2H-pyridin-5-yl]-1H-indol-4-yl]-2-fluoro-phenyl]piperidine-1-carboxylate (120 mg, 174 μmol) in DCM (3 mL) was added TFA (1.47 g, 12.8 mmol). Then the mixture was stirred at 25° C. for 2 hr. Upon completion, the reaction mixture was concentrated in vacuo to give the title compound (120 mg, 98% yield, TFA). LC-MS (ESI⁺) m/z 587.2 (M+H)⁺

### (R)-3-(4-(4-(4-chlorophenyl)piperidin-1-yl)-3-fluorophenyl)-3-methylpiperidine-2,6-dione (Intermediate SX) and (S)-3-(4-(4-(4-chlorophenyl)piperidin-1-yl)-3-fluorophenyl)-3-methylpiperidine-2,6-dione (Intermediate SY)

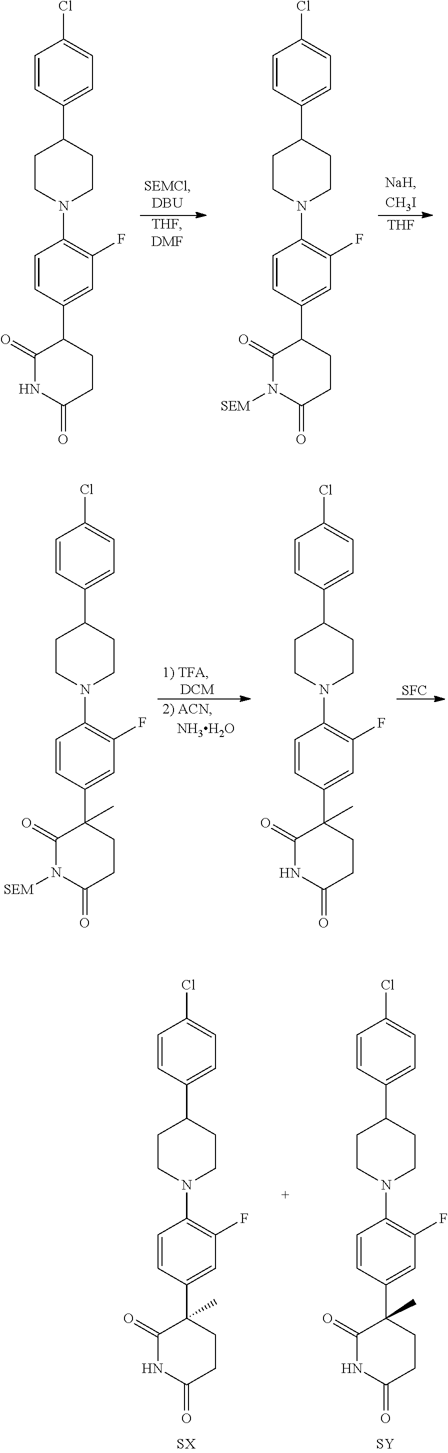

### Step 1—3-(4-(4-(4-Chlorophenyl)piperidin-1-yl)-3-fluorophenyl)-1-((2-(trimethylsilyl)ethoxy)methyl)piperidine-2,6-dione

To a solution of 3-[4-[4-(4-chlorophenyl)-1-piperidyl]-3-fluoro-phenyl]piperidine-2,6-dione (12 g, 21 mmol, Intermediate BT) in THF (120 mL) and DMF (20 mL) was added DBU (9.5 g, 62.8 mmol) and SEM-Cl (5.24 g, 31.4 mmol). The mixture was then stirred at 25° C. for 2 hrs. Upon completion, the reaction mixture was quenched with H₂O (100 mL) and extracted with ethyl acetate (100×3 mL). The combined organic layers were washed with brine (100 mL) and concentrated under reduced pressure to give a residue. The crude product was purified by reversed-phase HPLC (0.1% FA condition) to give the title compound (7 g, 57% yield) as a yellow solid. LC-MS (ESI⁺) m/z 531.2 (M+H)⁺.

### Step 2—3-(4-(4-(4-Chlorophenyl)piperidin-1-yl)-3-fluorophenyl)-3-methyl-1-((2-(trimethylsilyl)ethoxy)methyl)piperidine-2,6-dione

To a solution of 3-[4-[4-(4-chlorophenyl)-1-piperidyl]-3-fluoro-phenyl]-1-(2-trimethylsilylethoxymethyl)piperidine-2,6-dione (2.7 g, 5.1 mmol) in DMF (10 mL) was added tBuOK (1 M, 5.08 mL) at 0° C., and the mixture was stirred at 0° C. for 0.5 hr. Next, CH₃I (722 mg, 5.08 mmol) was added and the mixture was stirred at 0° C. for 2 hrs. Upon completion, the reaction mixture was quenched with aq. NH₄Cl (20 mL) and extracted with ethyl acetate (20 mL). The combined organic layers were washed with brine (20 mL), dried over Na₂SO₄, filtered and concentrated under reduced pressure to give a residue. The crude product was purified by reversed-phase HPLC (0.1% FA condition) to give the title compound (1 g, 35% yield) as a white solid. LC-MS (ESI⁺) m/z 545.6 (M+H)⁺.

### Step 3—3-(4-(4-(4-Chlorophenyl)piperidin-1-yl)-3-fluorophenyl)-3-methylpiperidine-2,6-dione

To a solution of 3-[4-[4-(4-chlorophenyl)-1-piperidyl]-3-fluoro-phenyl]-3-methyl-1-(2-trimethylsilylethoxymethyl)piperidine-2,6-dione (1 g, 2 mmol) in DCM (10 mL) was added TFA (4.61 g, 40.4 mmol) and the mixture was stirred at 25° C. for 1 hr. Next, the reaction mixture was concentrated under reduced pressure to remove TFA and ACN (10 mL) and NH₃·H₂O (6.40 mL, 25% solution) was added. The mixture was then stirred at 25° C. for 1 hr. On completion, the reaction mixture was concentrated under reduced pressure to give a residue. The crude product was purified by reversed-phase HPLC (0.1% FA condition) to give the title compound (2.3 g, 59% yield) as a yellow solid. LC-MS (ESI⁺) m/z 415.1 (M+H)⁺.

### Step 4—(R)-3-(4-(4-(4-chlorophenyl)piperidin-1-yl)-3-fluorophenyl)-3-methylpiperidine-2,6-dione and (S)-3-(4-(4-(4-chlorophenyl)piperidin-1-yl)-3-fluorophenyl)-3-methylpiperidine-2,6-dione

3-[4-[4-(4-Chlorophenyl)-1-piperidyl]-3-fluoro-phenyl]-3-methyl-piperidine-2,6-dione (0.7 g, 1.7 mmol) was separated by SFC (column: DAICEL CHIRALPAK AS (250 mm*30 mm, 10 um); mobile phase: [C02-i-PrOH]:B %:50%, isocratic elution mode) to give (R)-3-(4-(4-(4-chlorophenyl)piperidin-1-yl)-3-fluorophenyl)-3-methylpiperidine-2,6-dione (300 mg) as a white solid and (S)-3-(4-(4-(4-chlorophenyl)piperidin-1-yl)-3-fluorophenyl)-3-methylpiperidine-2,6-dione (300 mg) as a white solid. LC-MS (ESI⁺) m/z 415.1 (M+H)⁺ for both isomers. The absolute stereochemistry of the enantiomers was assigned arbitrarily.

### 1-(4-Chloro-2-fluoro-phenyl)-1,4-diazepane (Intermediate SZ)

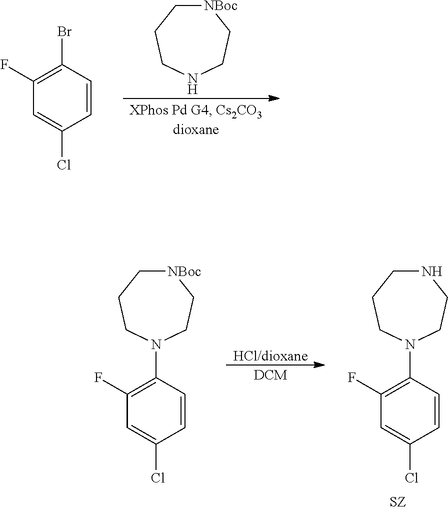

### Step 1—Tert-butyl 4-(4-chloro-2-fluorophenyl)-1,4-diazepane-1-carboxylate

A mixture of 1-bromo-4-chloro-2-fluoro-benzene (5 g, 20 mmol, CAS #1996-29-8), tert-butyl 1,4-diazepane-1-carboxylate (4.78 g, 23.9 mmol, 4.71 mL, CAS #112275-50-0), Xphos Pd G4 (3.08 g, 3.58 mmol), and Cs₂CO₃ (23.33 g, 71.62 mmol) in dioxane (120 mL) was degassed and purged with N₂ three times. Then the mixture was stirred at 100° C. for 12 hr under N₂ atmosphere. On completion, the reaction mixture was concentrated under reduced pressure to give a residue. The residue was purified by column chromatography (SiO₂, Petroleum ether:Ethyl acetate=30/1 to 10/1) to give the title compound (2 g, 20% yield) as an orange oil. LC-MS (ESI⁺) m/z 272.8 (M−55)⁺.

### Step 2—1-(4-Chloro-2-fluoro-phenyl)-1,4-diazepane

To a solution of tert-butyl 4-(4-chloro-2-fluoro-phenyl)-1,4-diazepane-1-carboxylate (1.5 g, 4.56 mmol) in HCl/dioxane (2 M, 5 mL) at 25° C., and the mixture was stirred at 25° C. for 1 hr. On completion, the reaction mixture was concentrated in vacuo to give the title compound (1.2 g, 99% yield, HCl) as a white solid. LC-MS (ESI⁺) m/z 229.1 (M+H)⁺.

### 3-[3-Fluoro-4-[4-[2-fluoro-4-(4,4,5,5-tetramethyl-1,3,2-dioxaborolan-2-yl)phenyl]-1,4-diazepan-1-yl]phenyl]piperidine-2,6-dione (Intermediate TA)

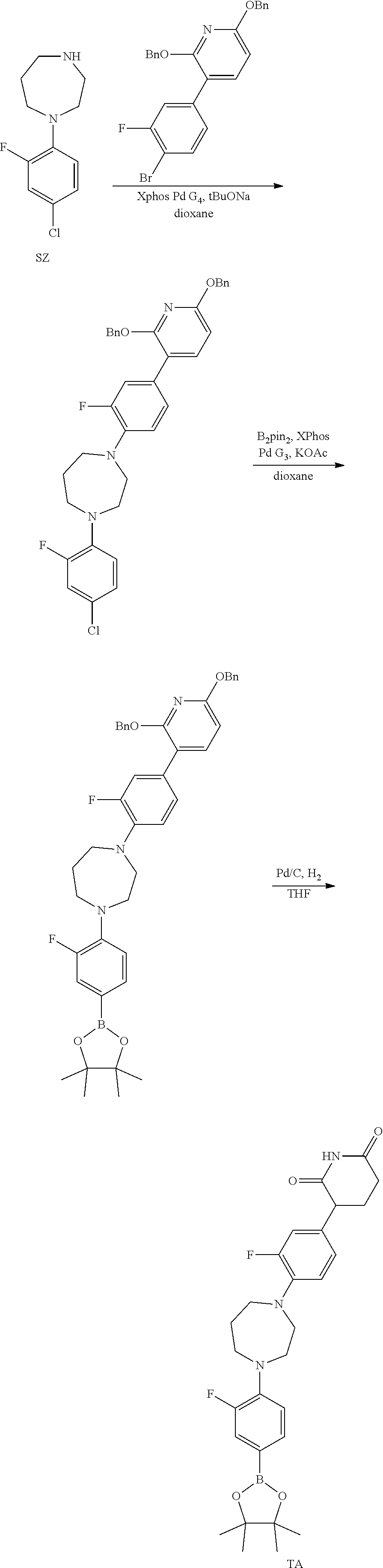

### Step 1—1-(4-Chloro-2-fluoro-phenyl)-4-[4-(2,6-dibenzyloxy-3-pyridyl)-2-fluoro-phenyl]-1,4-diazepane

A mixture of 1-(4-chloro-2-fluoro-phenyl)-1,4-diazepane (1.2 g, 4.53 mmol. HCl, Intermediate SZ), 2,6-dibenzyloxy-3-(4-bromo-3-fluoro-phenyl)pyridine (1.8 g. 3.88 mmol), Xphos Pd G₄ (500 mg, 581 μmol), tBuONa (1.86 g, 19.4 mmol) and 4 Å molecular seives (1 g, 3.88 mmol) in dioxane (20 mL) was degassed and purged with N₂ three times. Then the mixture was stirred at 100° C. for 1 hr under N₂ atmosphere. On completion, the reaction mixture was diluted with H₂O (20 mL) and extracted with ethyl acetate (50 mL×3). The combined organic layer was washed with brine (25 mL), dried over Na₂SO₄ and filtered. The filtrate was concentrated in vacuo to give a residue. The residue was purified by column chromatography (SiO₂, Petroleum ether/Ethyl acetate=1/0 to 0/1) to give the title compound (840 mg, 35% yield) as a yellow solid. LC-MS (ESI⁺) m/z 612.2 (M+H)⁺.

### Step 3—1-[4-(2,6-Dibenzyloxy-3-pyridyl)-2-fluoro-phenyl]-4-[2-fluoro-4-(4,4,5,5-tetramethyl-1,3,2-dioxaborolan-2-yl)phenyl]-1,4-diazepane

A mixture of 1-(4-chloro-2-fluoro-phenyl)-4-[4-(2,6-dibenzyloxy-3-pyridyl)-2-fluoro-phenyl]-1,4-diazepane (740 mg, 1.21 mmol), 4 Å molecular sieves (500 mg, 1.21 mmol), 4,4,5,5-tetramethyl-2-(4,4,5,5-tetramethyl-1,3,2-dioxaborolan-2-yl)-1,3,2-dioxaborolane (614 mg, 2.42 mmol), KOAc (356 mg, 3.63 mmol) and XPhos Pd G₃ (102 mg, 121 μmol) in dioxane (8 mL) was degassed and purged with N₂ three times. Then the mixture was stirred at 80° C. for 2 hr under N₂ atmosphere. On completion, the reaction mixture was diluted with H₂O (100 mL) and extracted with ethyl acetate (250 mL). The combined organic layer was washed with brine (250 mL), dried over Na₂SO₄ and filtered. The filtrate was concentrated in vacuo to give a residue. The residue was purified by column chromatography (SiO₂, Petroleum ether/Ethyl acetate=1/0 to 0/1) to give the title compound (600 mg, 71% yield) as a yellow solid. LC-MS (ESI⁺) m/z 704.1 (M+H)⁺.

### Step 4—3-[3-Fluoro-4-[4-[2-fluoro-4-(4,4,5,5-tetramethyl-1,3,2-dioxaborolan-2-yl)phenyl]-1,4-diazepan-1-yl]phenyl]piperidine-2,6-dione

To a solution of Pd/C (300 mg, 282 μmol, 10 wt %) in THF (3 mL) was added 1-[4-(2,6-dibenzyloxy-3-pyridyl)-2-fluoro-phenyl]-4-[2-fluoro-4-(4,4,5,5-tetramethyl-1,3,2-dioxaborolan-2-yl)phenyl]-1,4-diazepane (300 mg, 426 μmol). Then the reaction was de-grass with H₂ three times, and the mixture was stirred under H₂ (15 psi) at 25° C. for 12 hrs. On completion, the mixture was filtered and the filtrate was concentrated in vacuo to give a residue. The residue was triturated with ACN (10 mL) to give the title compound (125 mg, 55% yield) as a white solid. LC-MS (ESI⁺) m, z 526.2 (M+H).

### (R)-7-fluoro-N,N-dimethyl-6-(1-(2-methyl-3-(1H-pyrazol-1-yl)propanoyl)-1,2,5,6-tetrahydropyridin-3-yl)-4-(4,4,5,5-tetramethyl-1,3,2-dioxaborolan-2-yl)-1H-indole-2-carboxamide (Intermediate TB)

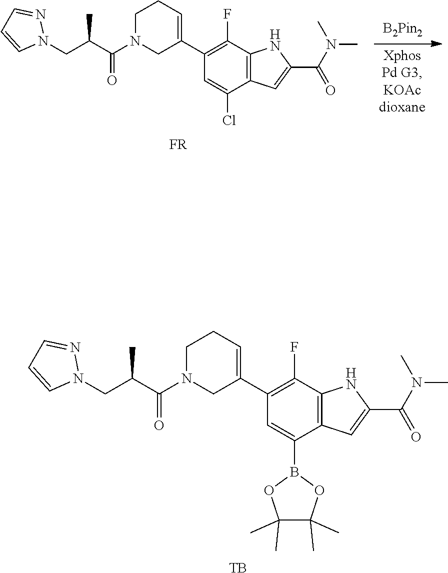 [SMILES: C[C@H](Cn1cccn1)C(=O)N1CCC=C(c2cc(Cl)c3cc(C(=O)N(C)C)[nH]c3c2F)C1 ; "FR" ; C[C@H](Cn1cccn1)C(=O)N1CCC=C(c2cc(B3OC(C)(C)C(C)(C)O3)c3cc(C(=O)N(C)C)[nH]c3c2F)C1 ; "TB"]

A mixture of (R)-4-chloro-7-fluoro-N,N-dimethyl-6-(1-(2-methyl-3-(1H-pyrazol-1-yl)propanoyl)-1,2,5,6-tetrahydropyridin-3-yl)-1H-indole-2-carboxamide (200 mg, 437 μmol, Intermediate FR), 4,4,5,5-tetramethyl-2-(4,4,5,5-tetramethyl-1,3,2-dioxaborolan-2-yl)-1,3,2-dioxaborolane (333 mg. 1.31 mmol), KOAc (129 mg, 1.31 mmol), XPhos Pd G3 (36.9 mg, 43.7 μmol) in dioxane (4 mL) was degassed and purged with N₂ three times. Then the mixture was stirred at 80° C. for 2 hrs under N₂ atmosphere. On completion, the reaction mixture was concentrated in vacuo to give the residue. The residue was purified by column chromatography (SiO₂, EA/THF=5/1) to give the title compound (150 mg, 46% yield) as a yellow solid. LC-MS (ESI⁺) m/z 550.6 (M+H)⁺.

### 7-Bromo-1-(cyclopropylmethyl)-N,N-dimethyl-2-[1-(3-pyrazol-1-ylpropanoyl)-3,6-dihydro-2H-pyridin-5-yl]indole-5-carboxamide (Intermediate TC)

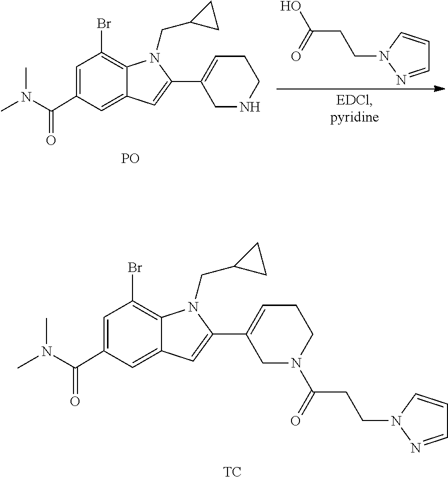 [SMILES: CN(C)C(=O)c1cc(Br)c2c(c1)cc(C1=CCCNC1)n2CC1CC1 ; O=C(O)CCn1cccn1 ; "PO" ; CN(C)C(=O)c1cc(Br)c2c(c1)cc(C1=CCCN(C(=O)CCn3cccn3)C1)n2CC1CC1 ; "TC"]

To a solution of 3-pyrazol-1-ylpropanoic acid (114 mg, 820 μmol, CAS #89532-73-0) in pyridine (4 mL) was added EDCI (262 mg, 1.37 mmol), then mixture was stirred at 25° C. Then 7-bromo-1-(cyclopropylmethyl)-N,N-dimethyl-2-(1,2,3,6-tetrahydropyridin-5-yl) indole-5-carboxamide (300 mg, 683 μmol, HCl, Intermediate PO) was added, and the mixture was stirred at 25° C. for 1 hr. On completion, the residue was diluted with water (200 mL) and extracted with EA (100 mL×3). The combined organic layer was dried over Na₂SO₄, filtered to give the filtrate and concentrated in vacuo to give a residue. The residue was purified by reverse phase (0.1% FA condition) to give the title compound (250 mg, 70% yield, FA) as yellow oil. LC-MS (ESI⁺) m/z 524.0 (M+H)⁺.

### 3-(1-Methyl-7-(4-(4-(4,4,5,5-tetramethyl-1,3,2-dioxaborolan-2-yl)phenyl)piperidin-1-yl)-1H-indazol-3-yl)piperidine-2,6-dione (Intermediate TD)

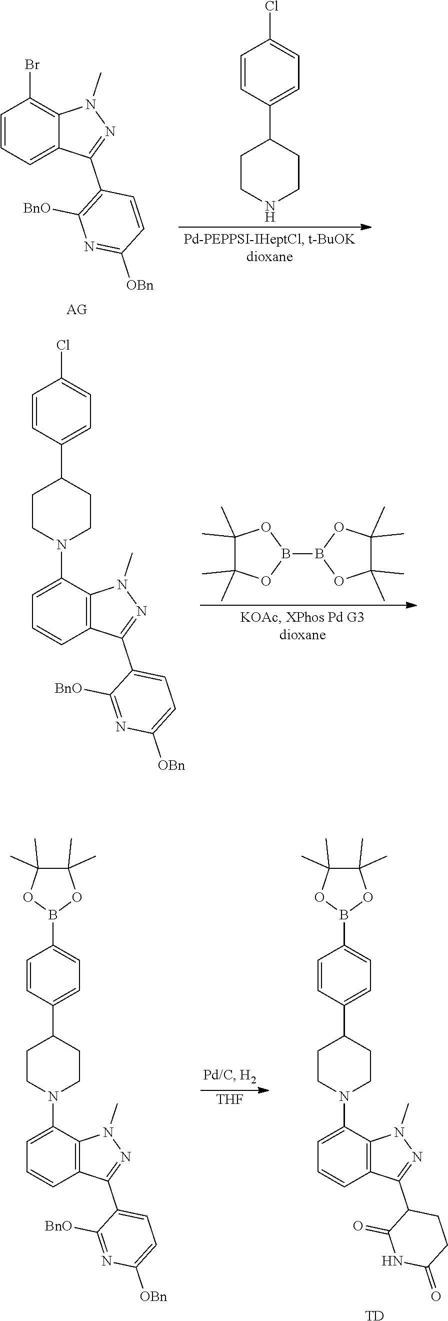

### Step 1—3-(2,6-Bis(benzyloxy)pyridin-3-yl)-7-(4-(4-chlorophenyl)piperidin-1-yl)-1-methyl-1H-indazole

To a solution of 7-bromo-3-(2,6-dibenzyloxy-3-pyridyl)-1-methyl-indazole (2.8 g, 5.6 mmol, Intermediate AG) in dioxane (50 mL) was added Pd-PEPPSI-IHeptCl (544 mg, 559 μmol) at 0° C., then 4-(4-chlorophenyl)piperidine (1.30 g, 5.60 mmol, CAS #26905-02-2) and t-BuONa (1.61 g, 16.7 mmol) was added. The mixture was then stirred at 100° C. for 2.5 hrs. On completion, the reaction mixture was diluted with NH₄Cl (50 mL) and extracted with ethyl acetate (100×3 mL). The combined organic layers were washed with brine (200 mL) and dried over anhydrous sodium sulfate, filtered and the filtrate was concentrated in vacuo to give the crude residue. Then the residue was purified by reverse phase flash (0.1% FA condition) to give the title compound (1.3 g, 37% yield) as a white solid. LC-MS (ESI⁺) m/z 615.3 (M+H)⁺.

### Step 2—3-(2,6-Bis(benzyloxy)pyridin-3-yl)-1-methyl-7-(4-(4-(4,4,5,5-tetramethyl-1,3,2-dioxaborolan-2-yl)phenyl)piperidin-1-yl)-1H-indazole

A mixture of 7-[4-(4-chlorophenyl)-1-piperidyl]-3-(2,6-dibenzyloxy-3-pyridyl)-1-methyl-indazole (1.3 g, 2.11 mmol), 4,4,5,5-tetramethyl-2-(4,4,5,5-tetramethyl-1,3,2-dioxaborolan-2-yl)-1,3,2-dioxaborolane (1.07 g, 4.23 mmol), KOAc (622 mg, 6.34 mmol), XPhos Pd G3 (178 mg, 211 μmol) and 4 Å molecular sieves (500 mg, 2.11 mmol) in dioxane (10 mL) was degassed and purged with N₂ three times. Then the mixture was stirred at 80° C. for 2 hrs under N₂ atmosphere. On completion, the reaction mixture was diluted with H₂O (100 mL) and extracted with ethyl acetate (100×3 mL). The combined organic layers were washed with brine (200 mL) and dried over anhydrous sodium sulfate, filtered and the filtrate was concentrated in vacuo to give the crude residue. Then the residue was purified by column chromatography (SiO₂, Petroleum ether:Ethyl acetate=10/1) to give the title compound (1.2 g, 72% yield) as a white solid. LC-MS (ESI⁺) m/z 707.3 (M+H)⁺

### Step 3—3-(1-Methyl-7-(4-(4-(4,4,5,5-tetramethyl-1,3,2-dioxaborolan-2-yl)phenyl)piperidin-1-yl)-1H-indazol-3-yl)piperidine-2,6-dione

To a solution of 3-(2,6-dibenzyloxy-3-pyridyl)-1-methyl-7-[4-[4-(4,4,5,5-tetramethyl-1,3,2-dioxaborolan-2-yl)phenyl]-1-piperidyl]indazole (1 g, 1 mmol) in THF (10 mL) was added Pd/C (1 g, 1 mmol, 10 wt %). The mixture was then stirred at 25° C. for 12 hrs under H₂ (15 Psi). On completion, the reaction mixture was filtered and concentrated in vacuo to give the title compound (740 mg) as a white solid. LC-MS (ESI⁺) m/z 529.2 (M+H)⁺.

### 2-(1-Acetyl-1,2,5,6-tetrahydropyridin-3-yl)-7-bromo-1-(cyclopropylmethyl)-N,N-dimethyl-1H-indole-5-carboxamide (Intermediate TE)

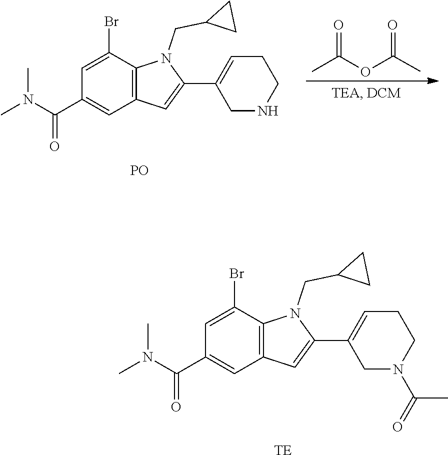

To a solution of 7-bromo-1-(cyclopropylmethyl)-N,N-dimethyl-2-(1,2,3,6-tetrahydropyridin-5-yl) indole-5-carboxamide (170 mg, 387 μmol, Intermediate PO) in DCM (3 mL) was added TEA (117 mg, 1.16 mmol) and acetyl acetate (59.3 mg, 581 μmol) and the mixture was stirred at 25° C. for 2 hrs. On completion, the reaction mixture was concentrated under reduced pressure to remove solvent. Then the crude product was purified by reverse phase (0.1% FA condition) to give the title compound (150 mg, 87% yield) as a white solid. LC-MS (ESI⁺) m/z 444.0 (M+H)⁺.

### 7-Bromo-2-(1-(tert-butoxycarbonyl)-1,2,5,6-tetrahydropyridin-3-yl)-4-fluoro-1H-indole-5-carboxylic acid (Intermediate TF)

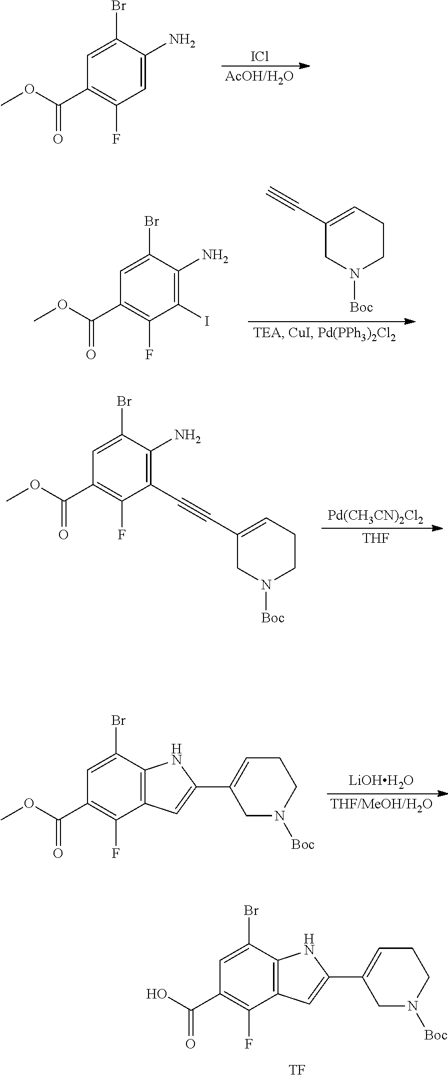

### Step 1—Methyl 4-amino-5-bromo-2-fluoro-3-iodobenzoate

To a solution of methyl 4-amino-5-bromo-2-fluoro-benzoate (2 g, 8 mmol, CAS #1427372-46-0) in HOAc (15 mL) and H₂O (15 mL) was added ClI (1.96 g, 12.1 mmol). The mixture was then stirred at 70° C. for 3 hrs. On completion, the reaction mixture was filtered and the filter cake was dried in vacuo to give the title compound (2.5 g) as a white solid. LC-MS (ESI⁺) m/z 373.8 (M+H)⁻; ¹H NMR (400 MHz, DMSO-d₆) δ=7.91 (d, J=7.6 Hz, 1H), 6.29 (s, 2H), 3.79 (s, 3H).

### Step 2—Tert-butyl3-((2-amino-3-bromo-6-fluoro-5-(methoxycarbonyl)phenyl)ethynyl)-5,6-dihydropyridine-1 (2H)-carboxylate

A mixture of methyl 4-amino-5-bromo-2-fluoro-3-iodo-benzoate (2.4 g, 6.4 mmol), tert-butyl 5-ethynyl-3,6-dihydro-2H-pyridine-1-carboxylate (2.66 g, 12.8 mmol), Pd(PPh₃)₂Cl₂ (450 mg, 641 μmol), CuI (122 mg, 641 μmol) and TEA (3.25 g, 32.1 mmol) in DMF (25 mL) was degassed and purged with N₂ three times. Then the mixture was stirred at 50° C. for 2 hrs under N₂ atmosphere. On completion, the reaction mixture was concentrated in vacuo to give the crude residue. The residue was purified by column chromatography (SiO₂, Petroleum ether:Ethyl acetate=15/1 to 10/1) to give the title compound (2 g, 63% yield) as a brown solid. LC-MS (ESI⁻) m/z 475.0 (M+Na)⁺; ¹H NMR (400 MHz, DMSO-d₆) δ=7.85 (br d, J=7.2 Hz, 1H), 6.64-6.28 (m, 3H), 4.01 (br s, 2H), 3.78 (s, 3H), 3.44 (br s, 2H), 2.24 (br s, 2H), 1.47-1.39 (m, 9H).

### Step 3—Methyl 7-bromo-2-(1-(tert-butoxycarbonyl)-1,2,5,6-tetrahydropyridin-3-yl)-4-fluoro-1H-indole-5-carboxylate

A mixture of tert-butyl 5-[2-(2-amino-3-bromo-6-fluoro-5-methoxycarbonyl-phenyl)ethynyl]-3,6-dihydro-2H-pyridine-1-carboxylate (2 g, 4 mmol), Pd(CH₃CN)₂Cl₂ (114 mg, 441 μmol) in THF (25 mL) was degassed and purged with N₂ three times. Then the mixture was stirred at 80° C. for 1 hr under N₂ atmosphere. On completion, the reaction mixture was concentrated under reduced pressure to give a residue. The residue was purified by column chromatography (SiO₂, Petroleum ether:Ethyl acetate=6/1 to 4/1) to give the title compound (1.4 g, 65% yield) as a white solid. ¹H NMR (400 MHz, DMSO-d₆) δ=11.74 (br s, 1H), 7.73 (br d, J=6.0 Hz. 1H), 6.92-6.70 (m, 2H), 4.27 (br s, 2H), 3.86 (s, 3H), 3.50 (br s, 2H), 2.32 (br d, J=1.2 Hz, 2H), 1.45 (s, 9H).

### Step 4—7-Bromo-2-(1-(tert-butoxycarbonyl)-1,2,5,6-tetrahydropyridin-3-yl)-4-fluoro-1H-indole-5-carboxylic acid

To a solution of methyl 7-bromo-2-(1-tert-butoxycarbonyl-3,6-dihydro-2H-pyridin-5-yl)-4-fluoro-TH-indole-5-carboxylate (1.4 g, 3.1 mmol) in MeOH (4 mL), THF (12 mL), and H₂O (4 mL) was added LiOH·H₂O (648 mg, 15.4 mmol). The mixture was then stirred at 50° C. for 12 hrs. On completion, the reaction mixture was added HCl (TN) until the pH was 5, then diluted with water (10 mL) and extracted with dichloromethane (3×30 mL). The combined organic layers were dried over anhydrous sodium sulfate, filtered and the filtrate was concentrated in vacuo to give the title compound (1.3 g) as an off-white solid. LC-MS (ESI⁺) m/z 439.0 (M+H)⁺.

### Tert-butyl 5-[7-bromo-5-(dimethylcarbamoyl)-4-fluoro-H-indol-2-yl]-3,6-dihydro-2H-pyridine-1-carboxylate (Intermediate TG)

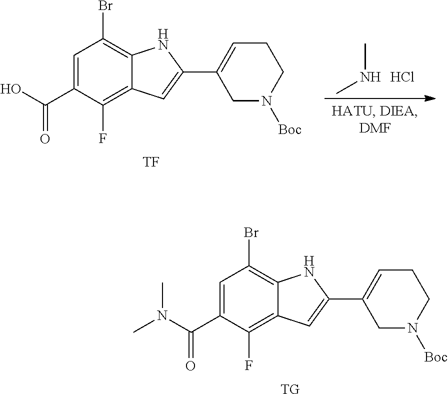

To a solution of 7-bromo-2-(1-tert-butoxycarbonyl-3,6-dihydro-2H-pyridin-5-yl)-4-fluoro-1H-indole-5-carboxylic acid (260 mg, 591 μmol, Intermediate TF) in DMF (5 mL) was added HATU (270 mg, 710 μmol) and DIEA (229 mg, 1.78 mmol), and N-methylmethanamine (96.5 mg, 1.18 mmol, HCl). The mixture was stirred at 25° C. for 1 hr. On completion, the reaction mixture was quenched with saturated NH₄Cl (20 mL) aqueous solution, and then extracted with EA (20 mL×3). The combined organic layers were washed with brine (20 mL), dried over Na₂SO₄ and filtered. The filtrate was concentrated in vacuo to give a residue. The residue was purified by column chromatography (SiO₂, petroleum ether/ethyl acetate=10/1 to 1/1) to give the title compound (260 mg, 84% yield) as a brown solid. ¹H NMR (400 MHz, DMSO-d₆) δ 11.52 (s, 1H), 7.23 (d, J=5.2 Hz, 1H), 6.83 (s, 1H), 6.65 (s, 1H), 4.26 (s, 2H), 3.49 (t, J=5.2 Hz, 2H), 3.01 (s, 3H), 2.89 (d, J=2.0 Hz, 3H), 2.31 (d, J=1.2 Hz, 2H), 1.44 (s, 9H); LC-MS (ESI⁺) m/z 467.9 (M+H)⁺.

### 7-Bromo-1-(cyclopropylmethyl)-4-fluoro-N,N-dimethyl-2-(1,2,3,6-tetrahydropyridin-5-yl) indole-5-carboxamide (Intermediate TH)

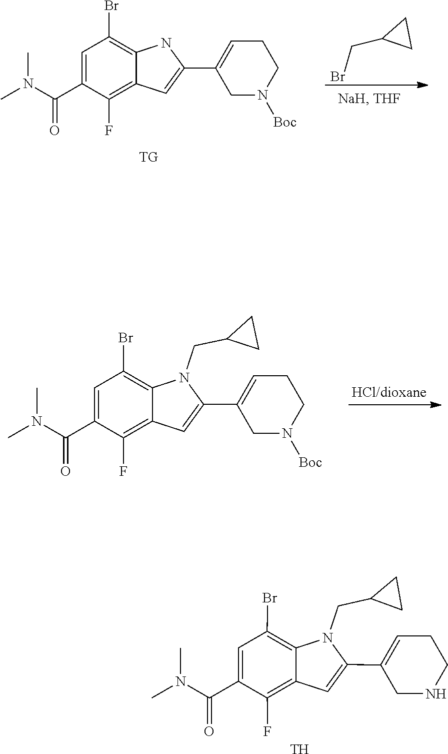

### Step 1—Tert-butyl 5-[7-bromo-1-(cyclopropylmethyl)-5-(dimethylcarbamoyl)-4-fluoro-indol-2-yl]-3,6-dihydro-2H-pyridine-1-carboxylate

A solution of tert-butyl 5-[7-bromo-5-(dimethylcarbamoyl)-4-fluoro-1H-indol-2-yl]-3,6-dihydro-2H-pyridine-1-carboxylate (260 mg, 557 μmol, Intermediate TG) in THF (5 mL) was degassed and purged with N₂ three times, and NaH (44.6 mg, 1.12 mmol, 60% dispersion in mineral oil) was added. After stirring at 0° C. for 0.5 h, bromomethylcyclopropane (225 mg, 1.67 mmol, CAS #7051-34-5) was added dropwise at 0° C. After addition, the mixture was stirred at 50° C. for 15.5 hrs. On completion, the reaction mixture was quenched by saturated NH₄Cl (20 mL) aqueous solution, and then extracted with EA (20 mL×3). The combined organic layers were washed with brine (20 mL), dried over Na₂SO₄ and filtered. The filtrate was concentrated in vacuo to give a residue. The residue was purified by column chromatography (SiO₂, petroleum ether/ethyl acetate=10/1 to 1/1) to give the title compound (210 mg, 68% yield) as a yellow solid. ¹H NMR (400 MHz, DMSO-d₆) δ 7.31 (d, J=6.0 Hz, 1H), 6.65 (s, 1H), 6.11 (s, 1H), 4.47 (d, J=6.8 Hz, 2H), 4.10 (s, 2H), 3.51 (t, J=5.2 Hz, 2H), 3.01 (s, 3H), 2.89 (s, 3H), 2.30 (d, J=2.4 Hz, 2H), 1.43 (s, 9H), 1.12-1.04 (m, 1H), 0.38 (d, J=7.6 Hz, 2H), 0.25-0.14 (m, 2H); LC-MS (ESI⁺) m/z 520.2 (M+H)⁺.

### Step 2—7-Bromo-1-(cyclopropylmethyl)-4-fluoro-N,N-dimethyl-2-(1,2,3,6-tetrahydropyridin-5-yl) indole-5-carboxamide

To a solution of tert-butyl 5-[7-bromo-1-(cyclopropylmethyl)-5-(dimethylcarbamoyl)-4-fluoro-indol-2-yl]-3,6-dihydro-2H-pyridine-1-carboxylate (160 mg, 307 μmol) in HCl/dioxane (3 mL) was stirred at 25° C. for 0.5 hrs. On completion, the reaction mixture was concentrated in vacuo to give the title compound (140 mg. 99% yield, HCl) as a yellow oil. LC-MS (ESI⁺) m/z 422.0 (M+H).

### 7-Bromo-1-(cyclopropylmethyl)-4-fluoro-N,N-dimethyl-2-[1-[3-(triazol-1-yl)propanoyl]-3,6-dihydro-2H-pyridin-5-yl]indole-5-carboxamide (Intermediate TI)

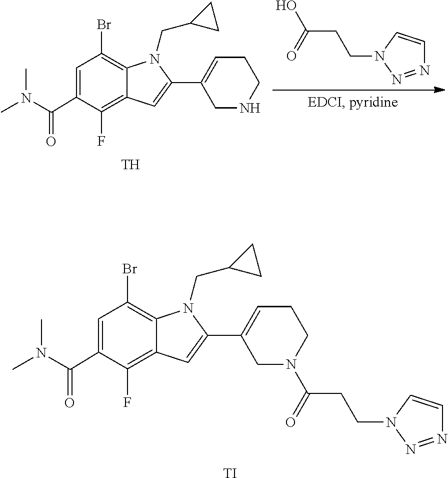

To a solution of 7-bromo-1-(cyclopropylmethyl)-4-fluoro-N,N-dimethyl-2-(1,2,3,6-tetrahydropyridin-5-yl) indole-5-carboxamide (140 mg, 306 μmol, HCl, Intermediate TH) in pyridine (2 mL) was added EDCI (88.1 mg, 459 μmol) and 3-(triazol-1-yl)propanoic acid (43.2 mg, 306 μmol, CAS #4332-45-0). The mixture was stirred at 25° C. for 1 hr. On completion, the reaction mixture was poured into H₂O (20 mL), and then extracted with EA (20 mL×3). The combined organic layers were washed with brine (20 mL), dried over Na₂SO₄ and filtered. The filtrate was concentrated in vacuo to give the title compound (160 mg. 91% yield) as a yellow solid. LC-MS (ESI⁺) m/z 543.1 (M+H)⁺.

### 3-[4-[4-(4-Chloro-2-fluoro-phenyl)-1-piperidyl]-2-fluoro-phenyl]piperidine-2,6-dione (Intermediate TJ)

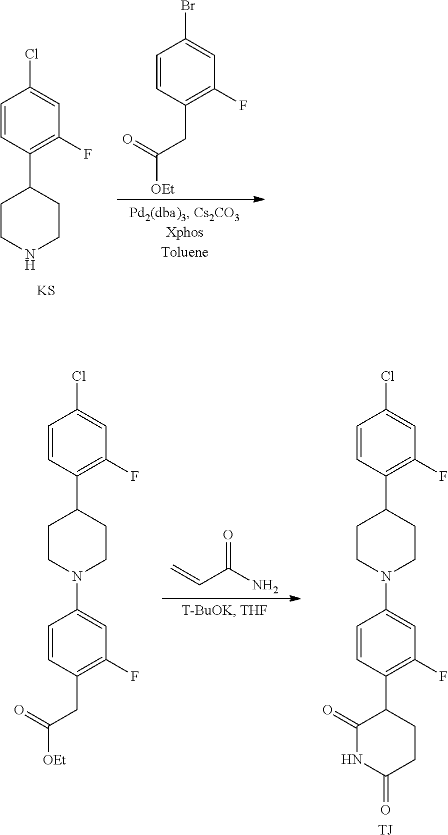

### Step 1—Ethyl 2-[4-[4-(4-chloro-2-fluoro-phenyl)-1-piperidyl]-2-fluoro-phenyl]acetate

To a solution of ethyl 2-(4-bromo-2-fluoro-phenyl)acetate (1.59 g, 6.08 mmol, CAS #924312-09-4) and 4-(4-chloro-2-fluoro-phenyl)piperidine (1.30 g, 6.08 mmol, Intermediate KS) in toluene (20 mL) was added XPhos (290 mg, 608 μmol), 4 Å molecular sieves (1 g, 6.08 mmol), Cs₂CO₃ (5.95 g, 18.3 mmol) and Pd₂(dba)₃ (279 mg, 304 μmol. Then the mixture was degassed under vacuum, purged with N₂ three times, and the mixture was stirred at 100° C. for 12 hrs under N₂ atmosphere. On completion, the reaction mixture was filtered with celite and quenched with water (60 mL). Then the mixture extracted with DCM (30 mL×3). The combined organic layers were washed with saturated NaCl (60 mL), dried over Na₂SO₄, filtered and concentrated in vacuo to give a residue. The residue was purified by column chromatography (SiO₂, PE/EA=100/1 to 4/1) to give the title compound (1.4 g, 89% yield) as yellow solid. ¹H NMR (400 MHz. DMSO-d₆) δ 7.84-7.75 (m, 1H), 7.51-7.44 (m, 1H), 7.38 (s, 1H), 7.24 (dd, J=1.2, 8.4 Hz, 1H), 7.13 (t, J=8.8 Hz, 1H), 6.77-6.73 (m, 1H), 4.07 (q, J=7.2 Hz, 2H), 3.84 (d, J=12.4 Hz, 2H), 3.56 (s, 2H), 3.03-2.91 (m, 1H), 2.80 (t, J=2.8, 12.0 Hz, 2H), 1.86-1.64 (m, 4H), 1.18 (t, J=7.2 Hz, 3H). LC-MS (ESI⁺) m/z 394.1 (M+H)⁺.

### Step 2—3-[4-[4-(4-Chloro-2-fluoro-phenyl)-1-piperidyl]-2-fluoro-phenyl]piperidine-2,6-dione

To a solution of ethyl 2-[4-[4-(4-chloro-2-fluoro-phenyl)-1-piperidyl]-2-fluoro-phenyl]acetate (560 mg, 1.27 mmol), 4 Å MS (500 mg) and prop-2-enamide (180 mg, 2.53 mmol) in DMF (5 mL) was added t-BuOK (1 M, 1.39 mL) at 0° C. Then the mixture was stirred at 0° C. for 0.2 hr. On completion, the mixture was slowly added HCl (2 M, 0.2 ml) at 0° C., then filtered and concentrated under reduced pressure to give a residue. The residue was purified by reverse phase (0.1% FA condition) to give the title compound (462 mg, 36% yield) as yellow solid. ¹H NMR (400 MHz. DMSO-d₆) δ 10.80 (s, 1H), 7.45-7.32 (m, 2H), 7.24 (dd, J=1.6, 8.4 Hz, 1H), 7.09 (t, J=8.8 Hz, 1H), 6.85-6.69 (m, 2H), 3.94-3.80 (m, 3H), 3.03-2.91 (m, 1H), 2.81 (dt, J=2.8, 12.0 Hz, 2H), 2.76-2.63 (m, 1H), 2.55-2.52 (m, 1H), 2.22-2.09 (m, 1H), 2.02-1.90 (m, 1H), 1.85-1.67 (m, 4H); LC-MS (ESI⁺) m/z 419.1 (M+H)⁺.

### 3-[2-Fluoro-4-[4-[2-fluoro-4-(4,4,5,5-tetramethyl-1,3,2-dioxaborolan-2-yl)phenyl]-1-piperidyl]phenyl]piperidine-2,6-dione (Intermediate TK)

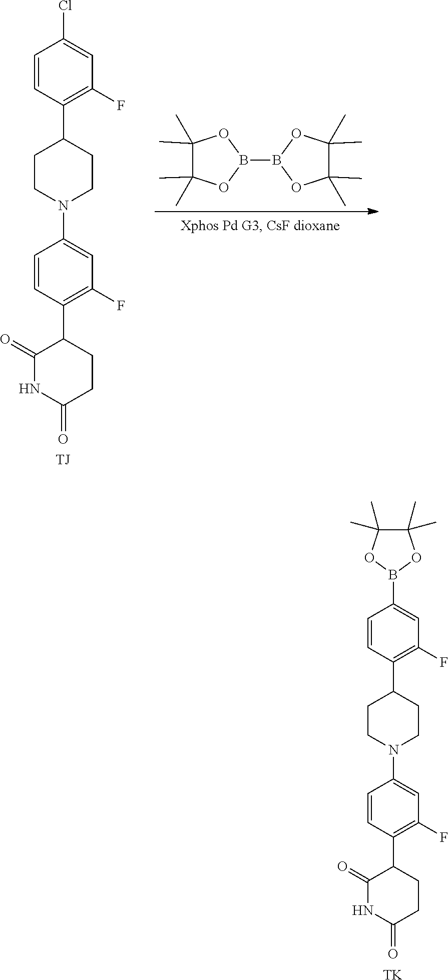

To a solution of 3-[4-[4-(4-chloro-2-fluoro-phenyl)-1-piperidyl]-2-fluoro-phenyl]piperidine-2,6-dione (200 mg, 396 μmol, Intermediate TJ) and 4,4,5,5-tetramethyl-2-(4,4,5,5-tetramethyl-1,3,2-dioxaborolan-2-yl)-1,3,2-dioxaborolane (252 mg, 991 μmol) in dioxane (2 mL) was added XPhos Pd G3 (33.6 mg, 39.6 μmol), 4 Å molecular sieves (100 mg), and CsF (181 mg, 1.19 mmol), and the mixture was degassed under vacuum, purged with N₂three times. Then the mixture was stirred at 80° C. for 12 hrs under N₂ atmosphere. On completion, the reaction mixture filtered with diatomaceous and was quenched with water (30 mL) and extracted with DCM (10 mL×3). The combined organic layers were washed with saturated NaCl (30 mL), dried over Na₂SO₄, filtered and concentrated in vacuo to give a residue. The residue was purified by column chromatography (SiO₂. PE/EA=100/0 to 3/1) to give the title compound (123 mg, 56% yield) as yellow solid. ¹H NMR (400 MHz, DMSO-d₆) δ 10.80 (s, 1H), 7.53-7.41 (m, 1H), 7.41-7.34 (m, 1H), 7.31 (d, J=10.8 Hz, 1H), 7.09 (t, J=8.8 Hz, 1H), 6.86-6.66 (m, 2H), 3.95-3.80 (m, 3H), 3.09-2.95 (m, 1H), 2.83 (t, J=10.4 Hz, 2H), 2.75-2.64 (m, 1H), 2.54 (s, 1H), 2.22-2.07 (m, 1H), 2.02-1.92 (m, 1H), 1.86-1.62 (m, 4H), 1.29 (s, 12H). LC-MS (ESI⁺) m/z 511.0 (M+H)⁺.

### (1S,5S)-3-(4-chloro-2-fluorophenyl)-3,6-diazabicyclo[3.2.0]heptane (Intermediate TL)

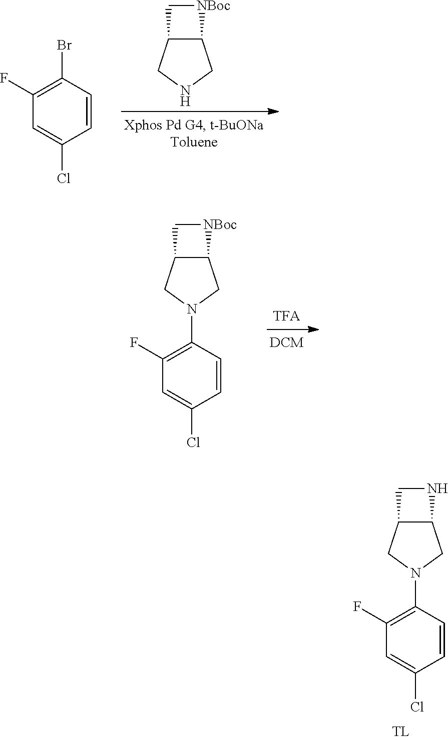 [SMILES: Fc1cc(Cl)ccc1Br ; CC(C)(C)OC(=O)N1C[C@H]2CNC[C@H]21 ; CC(C)(C)OC(=O)N1C[C@H]2CN(c3ccc(Cl)cc3F)C[C@H]21 ; Fc1cc(Cl)ccc1N1C[C@@H]2CN[C@@H]2C1]

### Step 1—(1R,5S)-tert-butyl 3-(4-chloro-2-fluorophenyl)-3,6-diazabicyclo[3.2.0]heptane-6-carboxylate

A mixture of 1-bromo-4-chloro-2-fluoro-benzene (1.5 g, 7.16 mmol, CAS #1996-29-8) tert-butyl (1R,5S)-3,6-diazabicyclo[3.2.0]heptane-6-carboxylate (947 mg, 4.77 mmol, CAS #799279-81-5), Xphos Pd G4 (411 mg, 477 μmol), and tBuONa (1.38 g, 14.3 mmol) in toluene (15 mL) was degassed and purged with N₂ three times. Then the mixture was stirred at 100° C. for 1 hr under N₂ atmosphere. On completion, the reaction mixture was filtered and concentrated under reduced pressure to give a residue. The residue was purified by column chromatography (SiO₂, Petroleum ether/Ethyl acetate=50/1 to 10/1) to give the title compound (1.1 g, 61% yield) as a white solid. LC-MS (ESI⁺) m/z 327.1 (M+H)⁺.

### Step 2—(1S,5S)-3-(4-chloro-2-fluorophenyl)-3,6-diazabicyclo[3.2.0]heptane

To a solution of tert-butyl (1R,5S)-3-(4-chloro-2-fluoro-phenyl)-3,6-diazabicyclo[3.2.0]heptane-6-carboxylate (950 mg, 2.91 mmol) in DCM (9 mL) was added TFA (4.61 g, 40.4 mmol, 3 mL). The mixture was then stirred at 25° C. for 0.5 hr. On completion, the reaction mixture was filtered and concentrated under reduced pressure to give a residue. The crude product was purified by reversed-phase HPLC (0.1% NH₃·H₂O) to give the title compound (650 mg, 99% yield) as a white solid. LC-MS (ESI+) m/z 227.1 (M+H)⁺.

### 3-(3-Fluoro-4-((1R,5S)-3-(2-fluoro-4-(4,4,5,5-tetramethyl-1,3,2-dioxaborolan-2-yl)phenyl)-3,6-diazabicyclo[3.2.0]heptan-6-yl)phenyl)piperidine-2,6-dione (Intermediate TM)

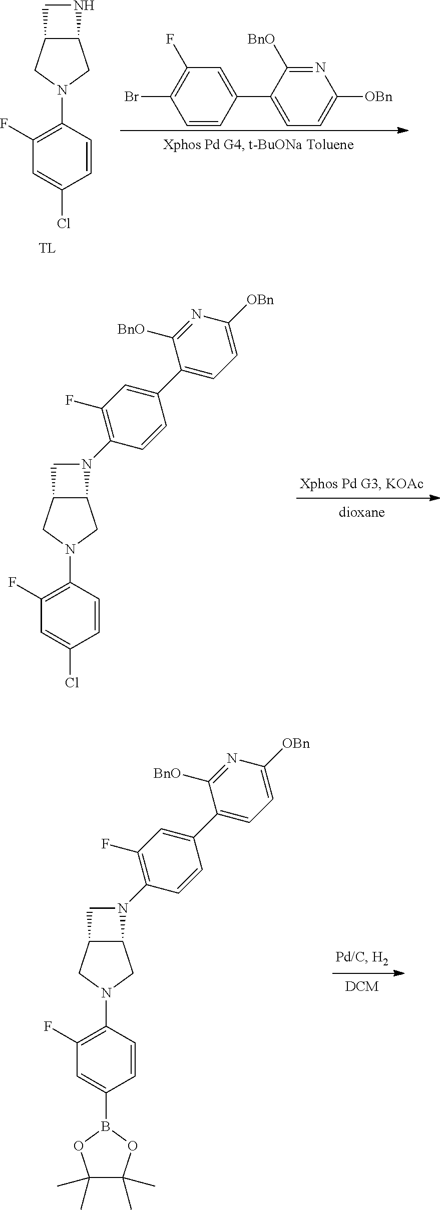 [SMILES: Fc1cc(Cl)ccc1N1C[C@@H]2CN[C@@H]2C1 ; Fc1cc(-c2ccc(OCc3ccccc3)nc2OCc2ccccc2)ccc1Br ; Fc1cc(Cl)ccc1N1C[C@@H]2CN(c3ccc(-c4ccc(OCc5ccccc5)nc4OCc4ccccc4)cc3F)[C@@H]2C1 ; CC1(C)OB(c2ccc(N3C[C@@H]4CN(c5ccc(-c6ccc(OCc7ccccc7)nc6OCc6ccccc6)cc5F)[C@@H]4C3)c(F)c2)OC1(C)C]

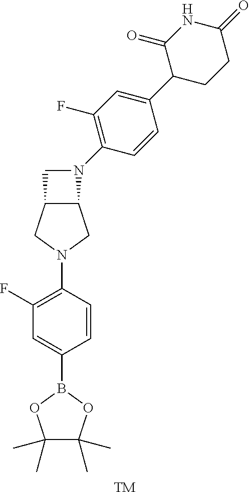

### Step 1—(1R,5S)-6-(4-(2,6-bis(benzyloxy)pyridin-3-yl)-2-fluorophenyl)-3-(4-chloro-2-fluorophenyl)-3,6-diazabicyclo[3.2.0]heptane

To a solution of (1S,5S)-3-(4-chloro-2-fluoro-phenyl)-3,6-diazabicyclo[3.2.0]heptane (600 mg, 2.65 mmol, Intermediate TL), and 2,6-dibenzyloxy-3-(4-bromo-3-fluoro-phenyl)pyridine (819 mg, 1.76 mmol) in toluene (8 mL) was added tBuONa (509 mg, 5.29 mmol) and Xphos Pd G4 (152 mg, 176 μmol). Then the mixture was stirred at 100° C. for 1 hr. On completion, the reaction mixture was filtered and concentrated under reduced pressure to give a residue. The residue was purified by column chromatography (SiO₂, Petroleum ether/Ethyl acetate=50/1 to 10/1) to give the title compound (1 g, 89% yield) as a white solid. ¹H NMR (400 MHz, DMSO-d₆) δ=7.71 (br d, J=8.1 Hz, 1H), 7.46-7.26 (m, 11H), 7.23 (br d. J=8.1 Hz, 1H), 7.19-7.08 (m, 1H), 7.06-6.64 (m, 2H), 6.60-6.47 (m, 2H), 5.38 (br d, J=18.3 Hz, 4H), 4.85 (br d, J=2.4 Hz, 1H), 4.04 (br t, J=7.6 Hz, 1H), 3.83-3.65 (m, 3H), 3.03-2.82 (m, 2H), 2.54 (s, 1H); LC-MS (ESI+) m/z 648.2 (M+H)⁺.

### Step 2—(1R,5S)-6-(4-(2,6-bis(benzyloxy)pyridin-3-yl)-2-fluorophenyl)-3-(2-fluoro-4-(4,4,5,5-tetramethyl-1,3,2-dioxaborolan-2-yl)phenyl)-3,6-diazabicyclo[3.2.0]heptane

A mixture of (1R,5S)-3-(4-chloro-2-fluoro-phenyl)-6-[4-(2,6-dibenzyloxy-3-pyridyl)-2-fluoro-phenyl]-3,6-diazabicyclo[3.2.0]heptane (0.9 g, 1.5 mmol), 4,4,5,5-tetramethyl-2-(4,4,5,5-tetramethyl-1,3,2-dioxaborolan-2-yl)-1,3,2-dioxaborolane (749 mg, 2.95 mmol), KOAc (434 mg, 4.43 mmol), XPhos Pd G3 (125 mg, 148 μmol) in dioxane (10 mL) was degassed and purged with N₂ three times. Then the mixture was stirred at 80° C. for 12 hrs under N₂ atmosphere. On completion, the reaction mixture was filtered and concentrated under reduced pressure to give a residue. The residue was purified by column chromatography (SiO₂, Petroleum ether/Ethyl acetate=50/1 to 10/1) to give the title compound (600 mg, 58% yield) as a white solid. LC-MS (ESI⁺) m/z 702.6 (M+H)⁺.

### Step 3—3-(3-Fluoro-4-((1R,5S)-3-(2-fluoro-4-(4,4,5,5-tetramethyl-1,3,2-dioxaborolan-2-yl)phenyl)-3,6-diazabicyclo[3.2.0]heptan-6-yl)phenyl)piperidine-2,6-dione

A mixture of (1R,5S)-6-[4-(2,6-dibenzyloxy-3-pyridyl)-2-fluoro-phenyl]-3-[2-fluoro-4-(4,4,5,5-tetramethyl-1,3,2-dioxaborolan-2-yl)phenyl]-3,6-diazabicyclo[3.2.0]heptane (0.3 g, 430 μmol), Pd/C (455 mg, 428 μmol, 10 wt %) in THE (10 mL) was degassed and purged with N₂ three times. Then the mixture was stirred at 25° C. for 12 hrs under H₂ atmosphere. On completion, the reaction mixture was filtered and the filtrate was concentrated under reduced pressure to give the title compound (270 mg) as a white solid. LC-MS (ESI⁺) m/z 523.9 (M+H)⁺.

### 3-Fluoro-2-methoxy-6-methyl-5-(1,2,3,6-tetrahydropyridin-4-yl)pyridine (Intermediate TN)

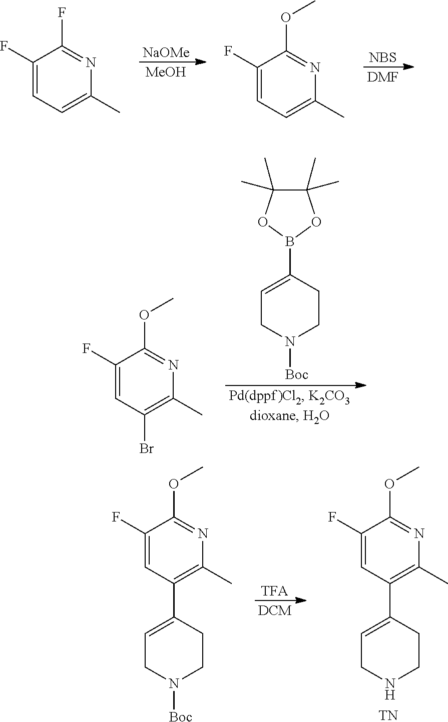

### Step 1—3-Fluoro-2-methoxy-6-methyl-pyridine

To a solution of 2,3-difluoro-6-methyl-pyridine (4.50 g, 34.8 mmol, CAS #1227579-04-5) in MeOH (40 mL) was added NaOMe (2.82 g, 52.3 mmol) at 0° C. The mixture was then stirred at 25° C. for 12 hrs. On completion, the solution was added into water (200 mL) and extracted with DCM (100 mL×3). The combined organic layers were washed with brine (100 mL), dried over anhydrous Na₂SO₄, filtered and concentrated in vacuo to give title compound (4.40 g, 87% yield) as colorless oil. ¹H NMR (400 MHz, DMSO-d₆) δ 7.52 (dd, J=8.0, 10.8 Hz, 1H), 6.82 (dd, J=2.8, 8.0 Hz, 1H), 3.90 (s, 3H), 2.36 (s, 3H).

### Step 2—5-Bromo-3-fluoro-2-methoxy-6-methyl-pyridine

To a solution of 3-fluoro-2-methoxy-6-methyl-pyridine (4.40 g, 31.1 mmol) in DMF (40 mL) was added NBS (6.66 g. 37.4 mmol) at 25° C., and the mixture was stirred at 25° C. for 1 hour. On completion, the solution was added into water (200 mL) and extracted with DCM (100 mL×3). The combined organic layers were washed with brine (100 mL), dried over anhydrous Na₂SO₄, filtered and concentrated in vacuo to give title compound (6.00 g, 88% yield) as yellow oil. ¹H NMR (400 MHz, DMSO-d₆) δ 8.00 (d, J=9.6 Hz, 1H), 3.92 (s, 3H), 2.45 (d, J=1.2 Hz, 3H).

### Step 3—Tert-butyl 4-(5-fluoro-6-methoxy-2-methyl-3-pyridyl)-3,6-dihydro-2H-pyridine-1-carboxylate

To a solution of 5-bromo-3-fluoro-2-methoxy-6-methyl-pyridine (5.00 g, 22.7 mmol) and tert-butyl 4-(4,4,5,5-tetramethyl-1,3,2-dioxaborolan-2-yl)-3,6-dihydro-2H-pyridine-1-carboxylate (7.03 g, 22.7 mmol, CAS #286961-14-6) in dioxane (50 mL) was added H₂O (2 mL), Pd(dppf)Cl₂·CH₂Cl₂ (1.86 g, 2.27 mmol) and Cs₂CO₃ (22.2 g, 68.2 mmol) at 25° C. Then the mixture was stirred at 80° C. for 2 hrs. On completion, the mixture was filtered and concentrated in vacuo. The residue was purified by column chromatography (SiO₂, Petroleum ether/Ethyl acetate=50/1 to 12/1) to give title compound (7.00 g, 92% yield) as colorless oil. ¹H NMR (400 MHz, DMSO-d₆) δ 7.45 (d, J=11.2 Hz, 1H), 5.66 (s, 1H), 3.95 (d, J=1.6 Hz, 2H), 3.92-3.88 (m, 3H), 3.57-3.49 (m, 2H), 2.33 (d, J=0.8 Hz, 3H), 2.30-2.24 (m, 2H), 1.43 (s, 9H); LC-MS (ESI⁺) m/z 323.1 (M+H)⁺.

### Step 4—3-Fluoro-2-methoxy-6-methyl-5-(1,2,3,6-tetrahydropyridin-4-yl)pyridine

To a solution of tert-butyl 4-(5-fluoro-6-methoxy-2-methyl-3-pyridyl)-3,6-dihydro-2H-pyridine-1-carboxylate (1.00 g, 3.10 mmol) in DCM (5 mL) was added TFA (3.07 g, 26.9 mmol) at 25° C. and the solution was stirred at 25° C. for 1 hr. On completion, the solution was quenched with NaHCO₃ aqueous solution (30 mL) and extracted with DCM (50 mL×3). The combined organic layers were washed with brine (50 mL), dried over anhydrous Na₂SO₄, filtered and concentrated in vacuo to give title compound (650 mg, 94% yield) as white solid. LC-MS (ESI⁺) m/z 194.0 (M+H)⁺.

### [5-[1-[4-(2,6-Dioxo-3-piperidyl)-2-fluoro-phenyl]-4-piperidyl]-3-fluoro-6-methyl-2-pyridyl]trifluoromethanesulfonate (Intermediate TO)

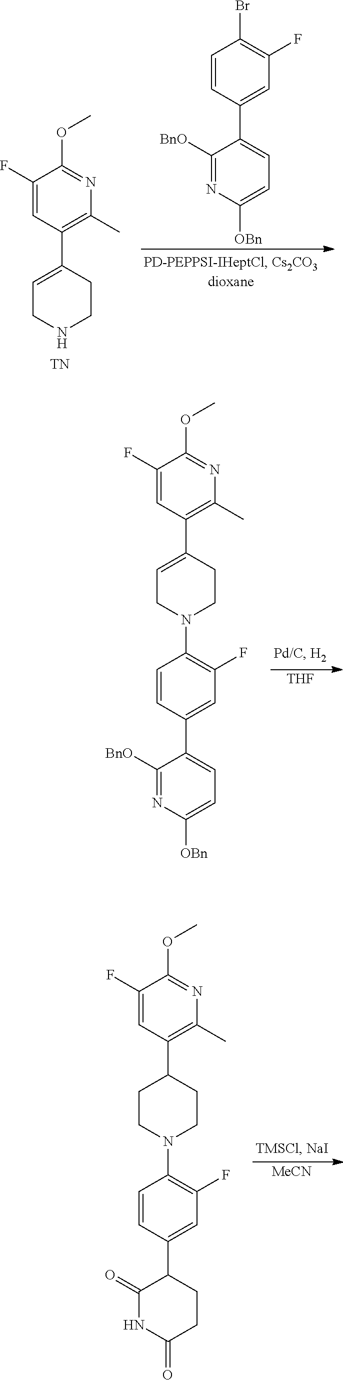

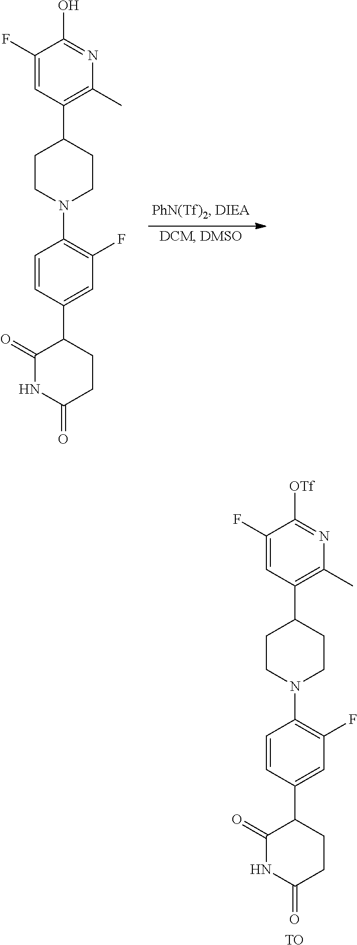

### Step 1—5-[1-[4-(2,6-Dibenzyloxy-3-pyridyl)-2-fluoro-phenyl]-3,6-dihydro-2H-pyridin-4-yl]-3-fluoro-2-methoxy-6-methyl-pyridine

To a solution of 3-fluoro-2-methoxy-6-methyl-5-(1,2,3,6-tetrahydropyridin-4-yl)pyridine (650 mg, 2.92 mmol, Intermediate TN) and 2,6-dibenzyloxy-3-(4-bromo-3-fluoro-phenyl)pyridine (1.36 g, 2.92 mmol) in dioxane (12 mL) was added Cs₂CO₃ (2.86 g, 8.77 mmol) and 1,3-bis[2,6-bis(1-propylbutyl)phenyl]-4,5-dichloro-2H-imidazol-1-ium-2-ide:3-chloropyridine dichloropalladium (284 mg, 292 μmol) at 25° C. The mixture was then stirred at 100° C. for 12 hrs. On completion, the mixture was filtered and concentrated in vacuo. The residue was purified by column chromatography (SiO₂, Petroleum ether/Ethyl acetate=20/1 to 10/1) to give title compound (600 mg, 33% yield) as white solid. ¹H NMR (400 MHz, CDCl₃) δ 7.61 (d, J=8.0 Hz, 1H), 7.48-7.27 (m, 12H), 7.13 (d, J=10.8 Hz, 1H), 7.03 (t, J=8.8 Hz, 1H), 6.48 (d, J=8.0 Hz, 1H), 5.74 (s, 1H), 5.45 (s, 2H), 5.38 (s, 2H), 4.02 (s, 3H), 3.82 (d, J=2.8 Hz, 2H), 3.42 (t, J=5.6 Hz, 2H), 2.49 (d, J=2.0 Hz, 2H), 2.43 (d, J=0.8 Hz, 3H).

### Step 2—3-[3-Fluoro-4-[4-(5-fluoro-6-methoxy-2-methyl-3-pyridyl)-1-piperidyl]phenyl]piperidine-2,6-dione

To a solution of 5-[1-[4-(2,6-dibenzyloxy-3-pyridyl)-2-fluoro-phenyl]-3,6-dihydro-2H-pyridin-4-yl]-3-fluoro-2-methoxy-6-methyl-pyridine (600 mg, 990 μmol) in THE (15 mL) was added Pd/C (0.5 g, 469 μmol, 10 wt %) under Ar₂. The suspension was degassed under vacuum and purged with H₂ several times. The mixture was stirred under H₂ (15 Psi) at 25° C. for 12 hrs. On completion, the mixture was filtered and concentrated in vacuo to give title compound (400 mg, 95% yield) as white solid. LC-MS (ESI⁺) m/z 430.3 (M+H)⁺.

### Step 3—3-[3-Fluoro-4-[4-(5-fluoro-6-hydroxy-2-methyl-3-pyridyl)-1-piperidyl]phenyl]piperidine-2,6-dione

To a solution of 3-[3-fluoro-4-[4-(5-fluoro-6-methoxy-2-methyl-3-pyridyl)-1-piperidyl]phenyl]piperidine-2,6-dione (300 mg, 698 μmol) in ACN (50 mL) was added NaI (314 mg, 2.10 mmol) and TMSCl (227 mg, 2.10 mmol). The reaction was then stirred at 80° C. for 2 hrs. On completion, the mixture was filtered and triturated with water (5 mL) at 25° C. for 1 hr to give title compound (280 mg, 96% yield) as yellow solid. ¹H NMR (400 MHz. DMSO-d₆) S 11.95 (d, J=1.2 Hz, 1H), 10.82 (s, 1H), 7.44 (d, J=12.8 Hz, 1H), 7.11-6.88 (m, 3H), 3.80 (dd, J=4.8, 11.6 Hz, 1H), 2.82-2.71 (m, 2H), 2.70-2.52 (m, 5H), 2.27-2.14 (m, 4H), 2.01 (dt, J=4.4, 8.8 Hz, 1H), 1.87-1.71 (m, 2H), 1.70-1.62 (m, 2H).

### Step 4—[5-[1-[4-(2,6-Dioxo-3-piperidyl)-2-fluoro-phenyl]-4-piperidyl]-3-fluoro-6-methyl-2-pyridyl]trifluoromethanesulfonate

To a solution of 3-[3-fluoro-4-[4-(5-fluoro-6-hydroxy-2-methyl-3-pyridyl)-1-piperidyl]phenyl]piperidine-2,6-dione (200 mg, 481 μmol) in DCM (2 mL) and DMSO (0.2 mL) was added 1,1,1-trifluoro-N-phenyl-N-(trifluoromethylsulfonyl)methanesulfonamide (206 mg, 578 μmol) and DIEA (12 mg, 962 μmol) at 25° C. The mixture was then stirred at 25° C. for 12 hrs. On completion, the mixture was quenched with water (10 mL) and extracted with ethyl acetate (20 mL×3). The combined organic layers were washed with brine (20 mL), dried over anhydrous Na₂SO₄, filtered and concentrated in vacuo. The residue was purified by column chromatography (SiO₂, Petroleum ether/Ethyl acetate=5/1 to 1/1) to give title compound (0.21 g, 71% yield) as white solid. ¹H NMR (400 MHz, DMSO-d₆) δ 10.83 (s, 1H), 8.20 (d, J=10.4 Hz, 1H), 7.10-6.84 (m, 3H), 3.88-3.71 (m, 1H), 3.47 (d, J=11.2 Hz, 3H), 3.01-2.74 (m, 3H), 2.71-2.59 (m, 1H), 2.53 (s, 3H), 2.21 (dd, J=3.2, 11.6 Hz, 1H), 2.04-1.96 (m, 1H), 1.87 (s, 4H).

### 2-Bromo-4-chloro-N,N-dimethyl-1,3-benzothiazole-6-carboxamide (Intermediate TP)

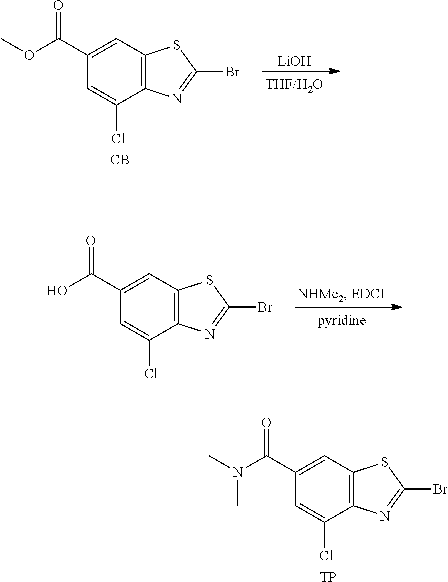

### Step 1—2-Bromo-4-chloro-1,3-benzothiazole-6-carboxylic acid

To a solution of methyl 2-bromo-4-chloro-1,3-benzothiazole-6-carboxylate (1.4 g, 4.6 mmol, Intermediate CB) in THF (16 mL) was added LiOH (1.09 g, 45.7 mmol) and H₂O (4 mL). The mixture was then stirred at 25° C. for 2 hr. On completion, the reaction mixture was added HCl (1M) and until the pH of aqueous layer was around 4, filtered and concentrated to give the title compound (1.1 g, 82% yield) as white solid. LC-MS (ESI⁺) m/z 293.9 (M+H)⁺.

### Step 2—2-Bromo-4-chloro-N,N-dimethyl-1,3-benzothiazole-6-carboxamide

To a solution of 2-bromo-4-chloro-1,3-benzothiazole-6-carboxylic acid (700 mg, 2.39 mmol) in pyridine (7 mL) was added EDCI (550 mg, 2.87 mmol) and N-methylmethanamine (195 mg, 2.39 mmol. HCl). The mixture was then stirred at 25° C. for 4 hr. On completion, the reaction mixture was diluted with H₂O (500 mL) and extracted with ethyl acetate (500 mL×3). The combined organic layer was washed with brine (250 mL), dried over Na₂SO₄ and filtered. The filtrate was concentrated in vacuo to give a residue. The residue was purified by column chromatography (SiO₂, Petroleum ether/Ethyl acetate-1/0 to 0/1) to give the title compound (500 mg, 65% yield) as a white solid. LC-MS (ESI⁺) m/z 321.0 (M+H)⁺.

### 4-Chloro-N,N-dimethyl-2-[1-[3-(triazol-1-yl)propanoyl]-3,6-dihydro-2H-pyridin-5-yl]-1,3-benzothiazole-6-carboxamide (Intermediate TO)

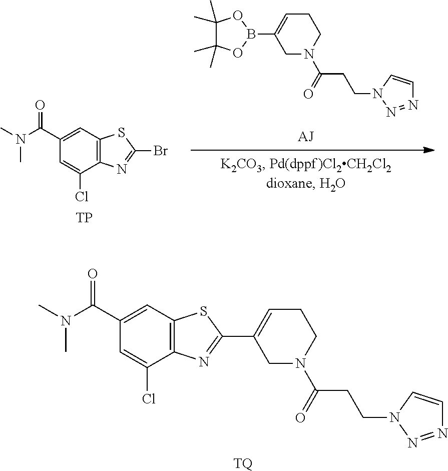

A mixture of 2-bromo-4-chloro-N,N-dimethyl-1,3-benzothiazole-6-carboxamide (450 mg, 1.41 mmol, Intermediate TP), 1-[5-(4,4,5,5-tetramethyl-1,3,2-dioxaborolan-2-yl)-3,6-dihydro-2H-pyridin-1-yl]-3-(triazol-1-yl)propan-1-one (468 mg, 1.41 mmol, Intermediate AJ), Pd(dppf)Cl₂·CH₂Cl₂ (114 mg, 141 μmol), K₂CO₃ (584 mg, 4.22 mmol) and H₂O (1 mL) in dioxane (5 mL) was degassed and purged with N₂ three times. Then the mixture was stirred at 80° C. for 12 hr under N₂ atmosphere. On completion, the reaction mixture was diluted with H₂O (50 mL) and extracted with ethyl acetate (100 mL, 3×). The combined organic layer was washed with brine (50 mL), dried over Na₂SO₄ and filtered. The filtrate was concentrated in vacuo to give a residue. The residue was purified by column chromatography (SiO₂, Petroleum ether/Ethyl acetate=1/0 to 0/1) to give the title compound (400 mg, 64% yield) as a white solid. LC-MS (ESI⁺) m/z 445.0 (M+H)⁺.

### Tert-butyl 4-(5-fluoro-6-methoxy-4-methyl-3-pyridyl)-3,6-dihydro-2H-pyridine-1-carboxylate (Intermediate TR)

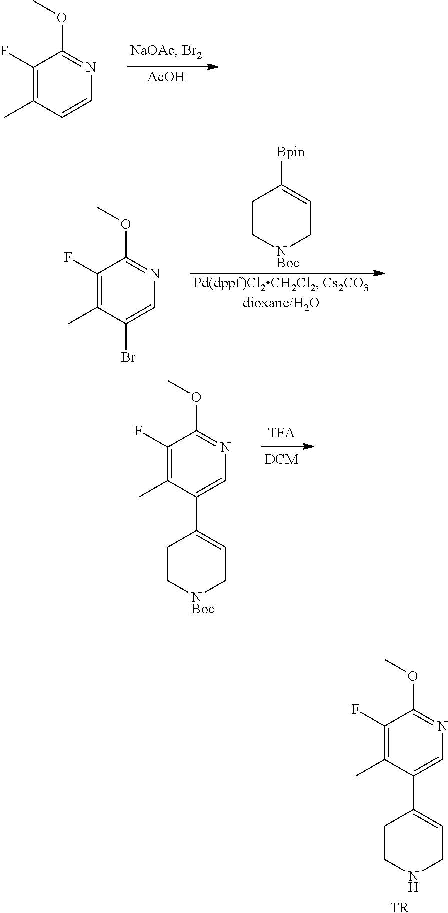

### Step 1—5-Bromo-3-fluoro-2-methoxy-4-methyl-pyridine

To a solution of 3-fluoro-2-methoxy-4-methyl-pyridine (4.5 g, 32 mmol, CAS #1227596-07-7) in AcOH (45 mL) was added NaOAc (5.23 g, 63.77 mmol) and Br₂ (10.1 g, 63.7 mmol) stirred at 80° C. for 2 hrs. On completion, the reaction mixture was added NaHCO₃ to pH=8 and extracted with ethyl acetate (80 mL×3). The combined organic layers were washed with brine (40 mL), dried over anhydrous sodium sulfate, filtered and the filtrate was concentrated in vacuo to give the residue. The residue was purified by silica gel chromatography (PE:EA=10:1) to give the title compound. ¹H NMR (400 MHz, DMSO-d₆) δ 8.10 (s, 1H), 3.91 (s, 3H), 2.27 (d, J=2.4 Hz, 3H): LC-MS (ESI⁺) m/z 221.9 (M+H)⁺.

### Step 2—Tert-butyl 4-(5-fluoro-6-methoxy-4-methyl-3-pyridyl)-3,6-dihydro-2H-pyridine-1-carboxylate

A mixture of 5-bromo-3-fluoro-2-methoxy-4-methyl-pyridine (3 g, 14 mmol), tert-butyl 4-(4,4,5,5-tetramethyl-1,3,2-dioxaborolan-2-yl)-3,6-dihydro-2H-pyridine-1-carboxylate (4.22 g. 13.6 mmol), Pd(dppf)Cl₂·CH₂Cl₂ (1.11 g, 1.36 mmol), and Cs₂CO₃ (8.88 g, 27.2 mmol) in dioxane (15 mL) and H₂O (3 mL) was degassed and purged with N₂ three times. Then the mixture was stirred at 80° C. for 2 hrs under N₂ atmosphere. On completion, the reaction mixture was quenched with water and extracted with EA (30 mL×3), and the combined layer was washed with brine and dried over Na₂SO₄, and concentrated in vacuo to give the residue. The residue was purified by column chromatography (SiO₂, Petroleum ether/Ethyl acetate=50/1 to 10/1) to give the title compound (3.6 g, 81% yield) as yellow solid. ¹H NMR (400 MHz, DMSO-d₆) δ 7.71 (s, 1H), 5.67 (s, 1H), 3.97 (s, 2H), 3.91 (s, 3H), 3.53 (t, J=5.6 Hz, 2H), 2.27 (d, J=1.6 Hz, 2H), 2.17 (d, J=2.4 Hz, 3H), 1.43 (s, 9H); LC-MS (ESI⁺) n/z 323.0 (M+H)⁺.

### Step 3—Tert-butyl 4-(5-fluoro-6-methoxy-4-methyl-3-pyridyl)-3,6-dihydro-2H-pyridine-1-carboxylate

To a solution of tert-butyl 4-(5-fluoro-6-methoxy-4-methyl-3-pyridyl)-3,6-dihydro-2H-pyridine-1-carboxylate (2 g, 6.20 mmol) in DCM (15 mL) was added TFA (707 mg. 6.20 mmol) and the mixture was stirred at 25° C. for 12 hr. On completion, the reaction mixture was concentrated in vacuo to give the residue. Then the residue was dissolved in DCM (20 mL) and NaHCO₃ (5 mL) was added, and the mixture was extracted with DCM (30 mL×3). The combined layer was washed with brine and dried over Na₂SO₄, and concentrated in vacuo to afford the title compound (1.3 g, 94% yield) as a yellow solid. LC-MS (ESI⁺) m/z 223.1 (M+H)⁺.

### [5-[1-[4-(2,6-Dioxo-3-piperidyl)-2-fluoro-phenyl]-4-piperidyl]-3-fluoro-4-methyl-2-pyridyl]trifluoromethanesulfonate (Intermediate TS)

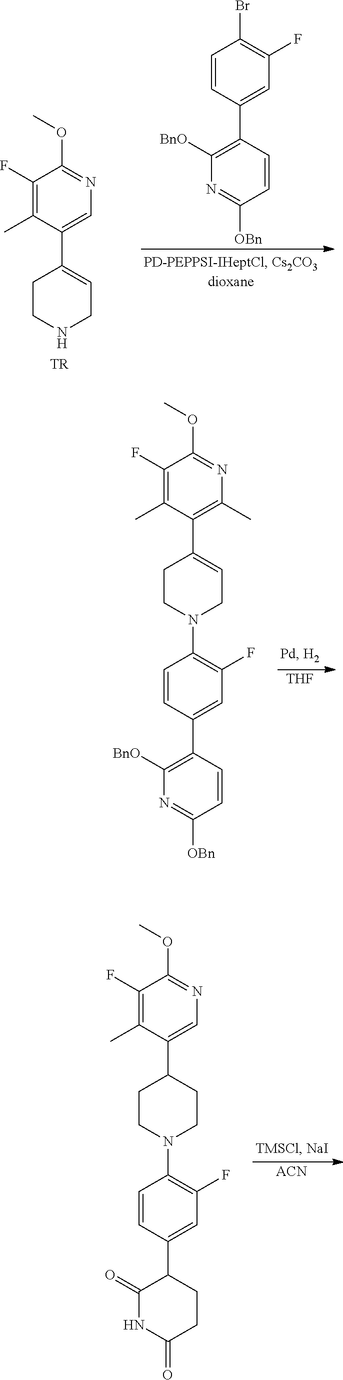

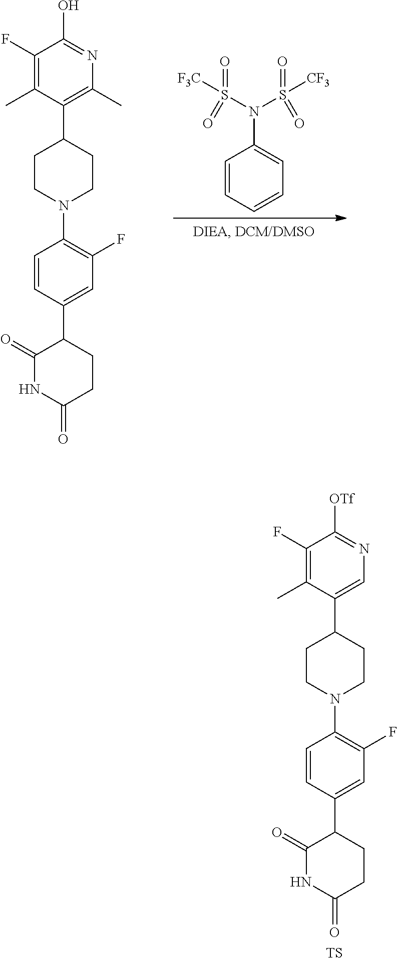

### Step 1—5-[1-[4-(2,6-Dibenzyloxy-3-pyridyl)-2-fluoro-phenyl]-3,6-dihydro-2H-pyridin-4-yl]-3-fluoro-2-methoxy-4-methyl-pyridine

A mixture of 3-fluoro-2-methoxy-4-methyl-5-(1,2,3,6-tetrahydropyridin-4-yl)pyridine (900 mg, 4.05 mmol, Intermediate TR), 2,6-dibenzyloxy-3-(4-bromo-3-fluoro-phenyl)pyridine (1.88 g, 4.05 mmol), 1,3-bis[2,6-bis(1-propylbutyl)phenyl]-4,5-dichloro-2H-imidazol-1-ium-2-ide; 3-chloropyridine dichloropalladium (393 mg, 404 μmol), Cs₂CO₃ (3.96 g. 12.1 mmol) and 4 Å molecular sieves (500 mg) in dioxane (10 mL) was degassed and purged with N₂ three times. Then the mixture was stirred at 100° C. for 12 hr under N₂ atmosphere. On completion, the reaction mixture was filtrated and concentrated in vacuo to give the residue. The residue was purified by column chromatography (SiO₂. Petroleum ether/Ethyl acetate=10/1 to 4/1) to give the title compound (1.23 g, 50% yield) as yellow solid. ¹H NMR (400 MHz, CDCl₃-d) δ 7.73 (s, 1H), 7.61 (d, J=8.0 Hz, 1H), 7.45-7.30 (m, 12H), 7.03 (t, J=8.8 Hz, 1H), 6.48 (d, J=8.0 Hz, 1H), 5.76 (td, J=1.6, 3.2 Hz, 1H), 5.45 (s, 2H), 5.38 (s, 2H), 4.03 (s, 3H), 3.84 (q, J=2.8 Hz, 2H), 3.43 (t, J=5.6 Hz, 2H), 2.49 (d, J=1.6 Hz, 2H), 2.26 (d, J=2.4 Hz, 3H); LC-MS (ESI⁺) m/z 606.0 (M+H)⁺.

### Step 2—3-[3-Fluoro-4-[4-(5-fluoro-6-methoxy-4-methyl-3-pyridyl)-1-piperidyl]phenyl]piperidine-2,6-dione

To a solution of 5-[1-[4-(2,6-dibenzyloxy-3-pyridyl)-2-fluoro-phenyl]-3,6-dihydro-2H-pyridin-4-yl]-3-fluoro-2-methoxy-4-methyl-pyridine (1.2 g, 2.0 mmol) in THF (20 mL) was added Pd/C (2.11 g, 1.98 mmol, 10 wt %) under Ar atmosphere. The suspension was degassed and purged with H₂ three times. Then the mixture was stirred under H₂ (15 Psi) at 25° C. for 7 hr. On completion, the reaction mixture was filtered through celite, and then concentrated in vacuo to give the title compound (801 mg, 94% yield) as yellow solid. ¹H NMR (400 MHz, CDCl₃-d) S 7.94 (s, 1H), 7.82 (s, 1H), 7.06-6.89 (m, 4H), 4.01 (s, 3H), 3.74 (dd. J=5.2, 9.8 Hz, 1H), 3.60 (d, J=12.0 Hz, 2H), 2.89-2.61 (m, 6H), 2.28-2.19 (m, 2H), 2.04-1.87 (m, 5H): LC-MS (ESI⁺) m/z 430.1 (M+H)⁺.

### Step 3—3-[3-Fluoro-4-[4-(5-fluoro-6-hydroxy-4-methyl-3-pyridyl)-1-piperidyl]phenyl]piperidine-2,6-dione

To a solution of 3-[3-fluoro-4-[4-(5-fluoro-6-methoxy-4-methyl-3-pyridyl)-1-piperidyl]phenyl]piperidine-2,6-dione (500 mg, 1.16 mmol) in ACN (5 mL) was added TMSCl (379 mg, 3.49 mmol) and NaI (523 mg, 3.49 mmol). The mixture was then stirred at 80° C. for 2 hr. On completion, the reaction mixture was filtered to give the filter cake. The filter cake was washed with H₂O (10 mL) to give the title compound (230 mg, 47% yield) as yellow solid. LC-MS (ESI⁺) m/z 416.1 (M+H)⁺.

### Step 4—[5-[1-[4-(2,6-Dioxo-3-piperidyl)-2-fluoro-phenyl]-4-piperidyl]-3-fluoro-4-methyl-2-pyridyl]trifluoromethanesulfonate

To a solution of 3-[3-fluoro-4-[4-(5-fluoro-6-hydroxy-4-methyl-3-pyridyl)-1-piperidyl]phenyl]piperidine-2,6-dione (210 mg, 505 μmol) in DMSO (1 mL) and DCM (1 mL) was added 1,1,1-trifluoro-N-phenyl-N-(trifluoromethylsulfonyl)methanesulfonamide (451 mg, 1.26 mmol, CAS #37595-74-7) and DIEA (261.33 mg, 2.02 mmol) and the mixture was stirred at 25° C. for 12 hr. On completion, the reaction mixture was quenched with H₂O (10 mL) and then extracted with EA and PE (3:1, 20 mL×3). The combined organic layer was washed with brine dried over Na₂SO₄ and concentrated in vacuo to give the residue. The residue was purified by column chromatography (SiO₂, Petroleum ether/Ethyl acetate=1/0 to 2/1) to give the title compound (130 mg, 46% yield) as yellow solid. LC-MS (ESI⁺) m, z 548.0 (M+H)⁺.

### 7-Bromo-1-(cyclopropylmethyl)-3-fluoro-N,N-dimethyl-2-(1,2,3,6-tetrahydropyridin-5-yl) indole-5-carboxamide (Intermediate TT)

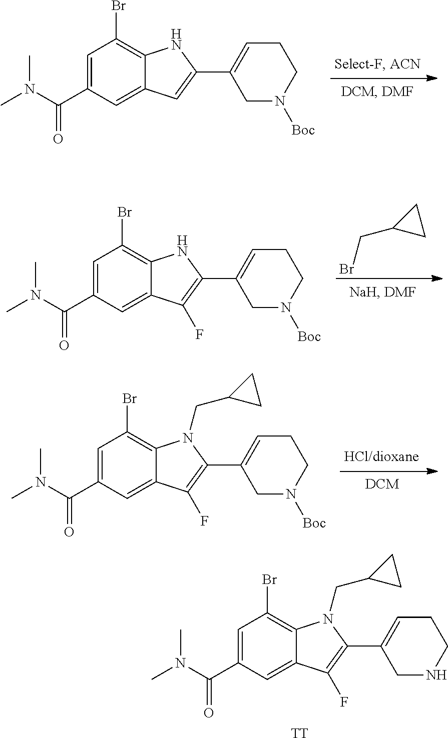 [SMILES: CN(C)C(=O)c1cc(Br)c2[nH]c(C3=CCCN(C(=O)OC(C)(C)C)C3)cc2c1 ; CN(C)C(=O)c1cc(Br)c2[nH]c(C3=CCCN(C(=O)OC(C)(C)C)C3)c(F)c2c1 ; BrCC1CC1 ; CN(C)C(=O)c1cc(Br)c2c(c1)c(F)c(C1=CCCN(C(=O)OC(C)(C)C)C1)n2CC1CC1 ; CN(C)C(=O)c1cc(Br)c2c(c1)c(F)c(C1=CCCNC1)n2CC1CC1 ; "TT"]

### Step 1—Tert-butyl 5-[7-bromo-5-(dimethylcarbamoyl)-3-fluoro-1H-indol-2-yl]-3,6-dihydro-2H-pyridine-1-carboxylate

To a solution of tert-butyl 5-[7-bromo-5-(dimethylcarbamoyl)-1H-indol-2-yl]-3,6-dihydro-2H-pyridine-1-carboxylate (2.5 g, 5.6 mmol, synthesized via Steps 1-7 of Intermediate PO) in ACN (10 mL), DCM (5 mL), and DMF (10 mL) was added Select F (1.28 g, 3.62 mmol) at 0° C. The reaction was stirred at 25° C. for 1 hr. On completion, the reaction was diluted with EA (100 mL). The organic layer was washed with water (100 mL×2), dried over Na₂SO₄ and concentrated in vacuo. The residue was purified by prep-HPLC (column: Daisogel C18 250*50 mm*8 um; mobile phase: [water (FA)-ACN]; gradient:35%-65% B over 20 min) to give the title compound (500 mg, 19% yield) as white solid. ¹H NMR (400 MHz, DMSO-d₆) δ 11.12 (s, 1H), 7.57 (d, J=1.2 Hz, 1H), 7.43 (d, J=1.2 Hz, 1H), 6.63 (s, 1H), 4.36 (s, 2H), 3.54-3.47 (m, 2H), 2.98 (s, 6H), 2.33 (s, 2H), 1.44 (s, 9H).

### Step 2—Tert-butyl 5-[7-bromo-1-(cyclopropylmethyl)-5-(dimethylcarbamoyl)-3-fluoro-indol-2-yl]-3,6-dihydro-2H-pyridine-1-carboxylate

A mixture of tert-butyl 5-[7-bromo-5-(dimethylcarbamoyl)-3-fluoro-1H-indol-2-yl]-3,6-dihydro-2H-pyridine-1-carboxylate (370 mg, 793 μmol) in THE (4 mL) was added NaH (63.4 mg, 1.59 mmol. 60% dispersion in mineral oil) at 0° C. under N₂. The reaction was added bromomethylcyclopropane (321 mg, 2.38 mmol) and stirred at 60° C. for 2 hrs. On completion, the reaction was quenched with NH4Cl solution (0.5 mL) and diluted with EA (10 mL). The organic layer was washed with water (10 mL), dried over Na₂SO₄ and concentrated in vacuo to give the title compound (410 mg, 99% yield) as yellow oil. ¹H NMR (400 MHz, DMSO-de) S 7.61 (d, J=1.6 Hz, 1H), 7.48 (d, J=1.2 Hz, 1H), 6.16 (s, 1H), 4.39 (d, J=6.4 Hz, 2H), 4.11 (s, 2H), 3.57-3.48 (m, 2H), 2.98 (s, 6H), 2.33 (d, J=2.0 Hz, 2H), 1.42 (s, 9H), 1.08-0.98 (m, 1H), 0.37 (d, J=6.4 Hz, 2H), 0.23-0.16 (m, 2H).

### Step 3—7-Bromo-1-(cyclopropylmethyl)-3-fluoro-N,N-dimethyl-2-(1,2,3,6-tetrahydropyridin-5-yl) indole-5-carboxamide

A mixture of tert-butyl 5-[7-bromo-1-(cyclopropylmethyl)-5-(dimethylcarbamoyl)-3-fluoro-indol-2-yl]-3,6-dihydro-2H-pyridine-1-carboxylate (420 mg, 807 μmol) in HCl/dioxane (2 M, 4.57 mL) and DCM (1 mL) was stirred at 25° C. for 1 hr. On completion, the reaction was concentrated in vacuo to give the title compound (368 mg, 99% yield, HCl) as yellow solid. LC-MS (ESI⁺) m/z 419.9 (M+H)⁺.

### 7-Bromo-1-(cyclopropylmethyl)-3-fluoro-N,N-dimethyl-2-[1-[3-(triazol-1-yl)propanoyl]-3,6-dihydro-2H-pyridin-5-yl]indole-5-carboxamide (Intermediate TU)

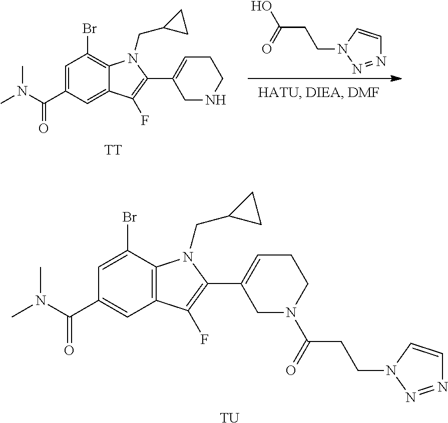 [SMILES: CN(C)C(=O)c1cc(Br)c2c(c1)c(F)c(C1=CCCNC1)n2CC1CC1 ; O=C(O)CCn1ccnn1 ; "TT" ; CN(C)C(=O)c1cc(Br)c2c(c1)c(F)c(C1=CCCN(C(=O)CCn3ccnn3)C1)n2CC1CC1 ; "TU"]

To a solution of 7-bromo-1-(cyclopropylmethyl)-3-fluoro-N,N-dimethyl-2-(1,2,3,6-tetrahydropyridin-5-yl) indole-5-carboxamide (164 mg, 359 μmol, HCl, Intermediate TT), 3-(triazol-1-yl)propanoic acid (50.6 mg, 359 μmol), DIEA (162 mg, 1.26 mmol) in DMF (3 mL) was added HATU (204 mg, 538 μmol) at 25° C., and stirred for 1 hr. On completion, the reaction was filtered. The residue was purified by prep-HPLC (column: CD05-Phenomenex luna C18 150*40*10 um; mobile phase: [water (FA)-ACN]; gradient: 27%-57% B over 25 min) to give the title compound (157 mg, 80% yield) as yellow solid. ¹H NMR (400 MHz, DMSO-d₆) δ 7.94-7.89 (m, 1H), 7.53-7.49 (m, 1H), 7.43 (d, J=1.2 Hz, 1H), 7.30 (d, J=1.2 Hz, 1H), 6.11-5.95 (m, 1H), 4.49-4.41 (m, 2H), 4.19 (d, J=6.4 Hz, 2H), 4.07-3.98 (m, 2H), 3.50-3.41 (m, 2H), 2.97-2.86 (m, 2H), 2.80 (s, 6H), 2.23-2.14 (m, 2H), 0.82 (dd, J=7.2, 11.8 Hz, 1H), 0.21-0.14 (m, 2H), 0.06-−0.02 (in, 2H).

### 6-(1-(3-(1H-pyrazol-1-yl)propanoyl)-1,2,5,6-tetrahydropyridin-3-yl)-7-fluoro-N,N-dimethyl-4-(4-(piperidin-4-yl)phenyl)-1H-indole-2-carboxamide (Intermediate TV)

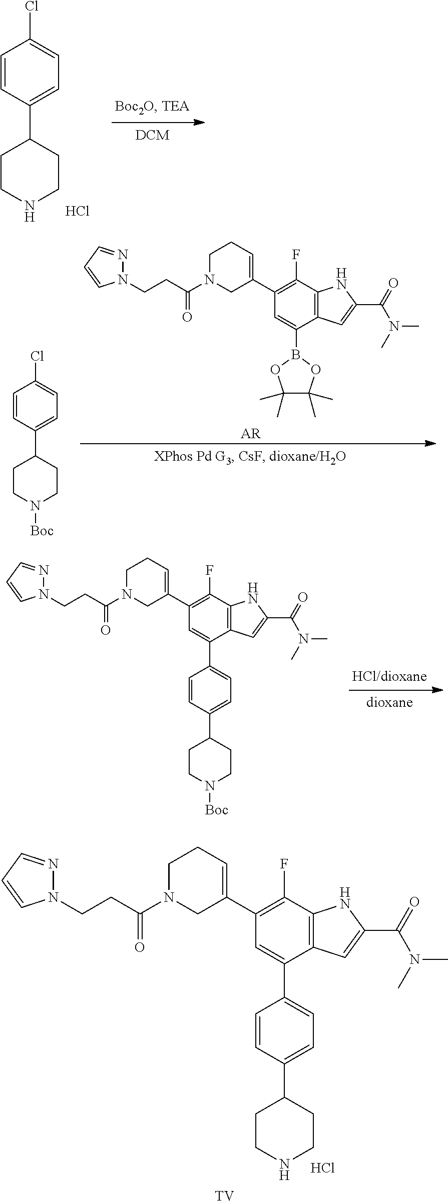

### Step 1—Tert-butyl 4-(4-chlorophenyl)piperidine-1-carboxylate

To a solution of 4-(4-chlorophenyl)piperidine (1 g. 4 mmol, HCl) in DCM (10 mL) was added TEA (1.31 g, 12.9 mmol) and Boc₂O (1.41 g. 6.46 mmol), then mixture was stirred at 25° C. for 12 hrs. On completion, the reaction mixture was quenched with water (30 mL) and extracted with ethyl acetate (30 mL×3). The combined organic layers were washed with saturated NaCl (30 mL×2), dried over Na₂SO₄, filtered and concentrated in vacuo to give a residue. The residue was purified by column chromatography (SiO₂, PE/EA=100/0 to 70/30) to give the title compound (0.9 g, 70% yield) as colorless oil. ¹H NMR (400 MHz. DMSO-d₆) δ 7.35-7.32 (m, 2H), 7.28-7.26 (m, 2H), 4.07-4.02 (m, 2H), 2.90-2.50 (m, 3H), 1.73 (d, J=12.8 Hz, 2H), 1.50-1.43 (m, 2H), 1.41 (s, 9H): LC-MS (ESI⁺) m/z 239.9 (M+H)⁺.

### Step 2—Tert-butyl 4-(4-(6-(1-(3-(1H-pyrazol-1-yl)propanoyl)-1,2,5,6-tetrahydropyridin-3-yl)-2-(dimethylcarbamoyl)-7-fluoro-1H-indol-4-yl)phenyl)piperidine-1-carboxylate

To a solution of 6-(1-(3-(1H-pyrazol-1-yl)propanoyl)-1,2,5,6-tetrahydropyridin-3-yl)-7-fluoro-N,N-dimethyl-4-(4,4,5,5-tetramethyl-1,3,2-dioxaborolan-2-yl)-1H-indole-2-carboxamide (0.5 g, 0.93 mmol, Intermediate AR) in dioxane (5 mL) was added tert-butyl 4-(4-chlorophenyl)piperidine-1-carboxylate (276 mg, 0.934 mmol), XPhos Pd G₃ (79.0 mg, 0.934 mmol), CsF (425 mg, 2.80 mmol) and H₂O (1 mL), then mixture was stirred at 80° C. for 2 hrs under N₂ atmosphere. On completion, the reaction mixture was quenched with water (30 mL) and extracted with ethyl acetate (30 mL×3). The combined organic layers were washed with brine (30 mL×2), dried over Na₂SO₄, filtered and concentrated in vacuo to give a residue. The residue was purified by column chromatography (SiO₂, PE/EA=100/0 to 20/80) to give the title compound (430 mg, 68% yield) as white solid. ¹H NMR (400 MHz, DMSO-d₆) δ 12.18 (d, J=8.8 Hz, 1H), 7.73-7.70 (m, 1H), 7.60 (d, J=7.6 Hz, 2H), 7.37 (d, J=8.0 Hz, 2H), 7.05-6.98 (m, 1H), 6.86 (s, 1H), 6.21-6.14 (m, 2H), 4.39-4.30 (m, 4H), 4.11-4.05 (m, 2H), 3.66-3.56 (m, 2H), 3.22-3.02 (m, 6H), 3.00-2.94 (m, 3H), 2.86-2.72 (m, 3H), 2.33-2.26 (m, 2H), 1.81 (d, J=11.6 Hz, 2H), 1.58-1.48 (m, 2H), 1.43 (s, 9H); LC-MS (ESI⁺) m/z 669.1 (M+H)⁺.

### Step 3—6-(1-(3-(1H-pyrazol-1-yl)propanoyl)-1,2,5,6-tetrahydropyridin-3-yl)-7-fluoro-N,N-dimethyl-4-(4-(piperidin-4-yl)phenyl)-1H-indole-2-carboxamide

To a solution of 6-(1-(3-(1H-pyrazol-1-yl)propanoyl)-1,2,5,6-tetrahydropyridin-3-yl)-7-fluoro-N,N-dimethyl-4-(4-(piperidin-4-yl)phenyl)-1H-indole-2-carboxamide (300 mg, 448 mmol) in dioxane (1.5 mL) was HCl/dioxane (2 M, 1.5 mL), then mixture was stirred at 25° C. for 0.5 hr. On completion, the reaction mixture was concentrated under reduced pressure to give the title compound (270 mg, HCl) as yellow oil. LC-MS (ESI⁺) m/z 569.2 (M+H)⁺.

### 3-(3-Fluoro-4-(4-(3-methyl-4-(4,4,5,5-tetramethyl-1,3,2-dioxaborolan-2-yl)phenyl) piperidin-1-yl)phenyl)piperidine-2,6-dione (Intermediate TW)

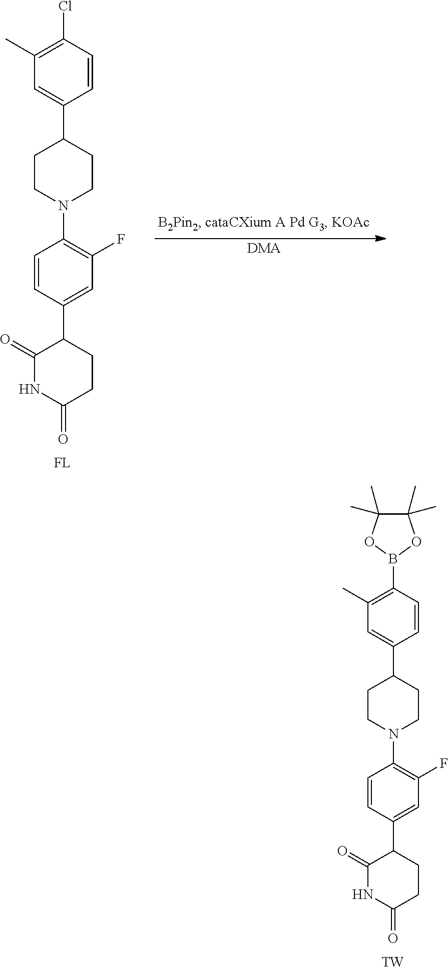

To a solution of 3-(4-(4-(4-chloro-3-methylphenyl)piperidin-1-yl)-3-fluorophenyl)piperidine-2,6-dione (195 mg, 470 μmol, Intermediate FL) in DMA (2 mL) was added 4,4,4′,4′,5,5,5′,5′-octamethyl-2,2′-bi(1,3,2-dioxaborolane) (238 mg, 940 μmol), KOAc (184 mg, 1.88 mmol) and cataCXium A Pd G₃ (34.2 mg, 47.0 μmol), then the mixture was stirred at 80° C. for 16 hrs under N₂ atmosphere. On completion, the reaction mixture was quenched with water (20 mL) and extracted with ethyl acetate (30 mL×3). The combined organic layers were washed with brine (30 mL×2), dried over Na₂SO₄, filtered and concentrated in vacuo to give a residue. The residue was purified reversed-phase flash (0.1% FA condition) to give the title compound (140 mg, 53% yield) as white solid. ¹H NMR (400 MHz, DMSO-d₆) δ 10.81 (s, 1H), 7.58 (d, J=7.6 Hz, 1H), 7.11-7.08 (m, 2H), 7.05-7.01 (m, 2H), 6.98-6.96 (m, 1H), 3.81 (dd, J=4.8, 11.6 Hz, 1H), 3.44 (d, J=11.6 Hz, 2H), 2.79-2.73 (m, 2H), 2.67-2.61 (m, 2H), 2.45 (s, 3H), 2.25-2.15 (m, 1H), 2.03-1.99 (m, 1H), 1.90 (s, 1H), 1.83-1.76 (m 4H), 1.29 (s, 12H); LC-MS (ESI⁺) m/z 507.3 (M+H)⁺.

### 3-[4-[2-[4-(4-Chloro-2,3-difluoro-phenyl)-1-piperidyl]ethyl]-3,3-dimethyl-2-oxo-indolin-1-yl] piperidine-2,6-dione (Intermediate TX)

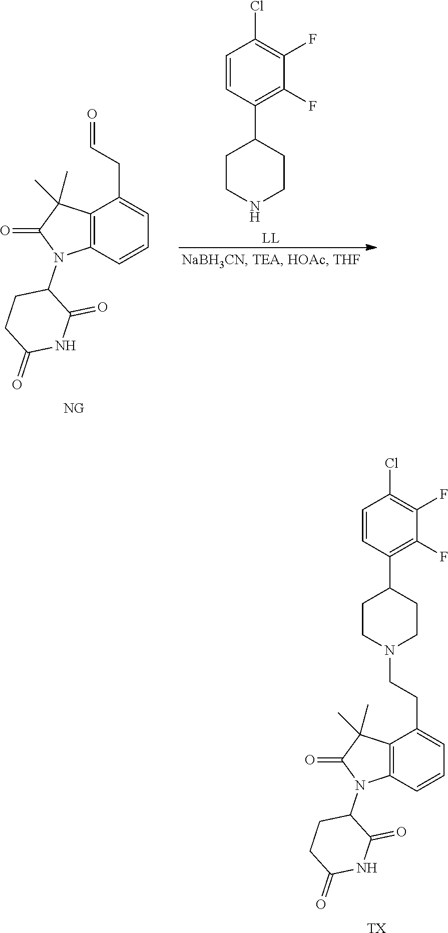

To a solution of 4-(4-chloro-2,3-difluoro-phenyl)piperidine (89 mg, 384 μmol, Intermediate LL) in THF (2 mL) was added TEA (38.9 mg, 384 μmol), followed by 2-[1-(2,6-dioxo-3-piperidyl)-3,3-dimethyl-2-oxo-indolin-4-yl]acetaldehyde (150 mg, 384 μmol, HCl, Intermediate NG). Then HOAc (23 mg, 384 μmol) was added to the mixture until the pH=5 and the mixture was stirred at 25° C. for 30 min. Next, NaBH₃CN (36 mg, 577 μmol) was added at 0° C. slowly and the mixture was stirred at 25° C. for 2 hrs. On completion, the reaction mixture was quenched with H₂O (2 mL), and then extracted with EA (10 mL×3). The combined organic layer was washed with brine, dried over Na₂SO₄ and concentrated in vacuo to give the residue. The residue was purified by reverse phase (0.1% FA condition) to give the title compound (100 mg, 49% yield) as white solid. LC-MS (ESI⁺) m/z 530.1 (M+H)⁺.

### Example 1 (Method 1): Synthesis of 5-((S)-1-(3-(1H-pyrazol-1-yl)propanoyl)piperidin-3-yl)-7-(4-(4-(3-chloro-4-(2,6-dioxopiperidin-3-yl)phenethyl)piperazin-1-yl)phenyl)-4-fluoro-N,N-dimethylbenzofuran-2-carboxamide (I-281)

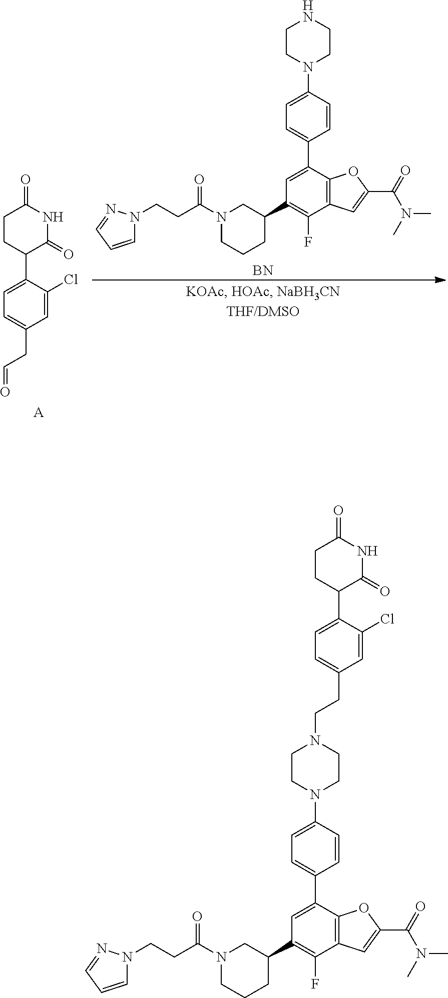

To a solution of (S)-5-(1-(3-(1H-pyrazol-1-yl)propanoyl)piperidin-3-yl)-4-fluoro-N,N-dimethyl-7-(4-(piperazin-1-yl)phenyl)benzofuran-2-carboxamide (80 mg, 139 μmol, Intermediate BN) and 2-[3-chloro-4-(2,6-dioxo-3-piperidyl)phenyl]acetaldehyde (55.6 mg, 209 μmol, Intermediate A) in THF (1 mL) and DMSO (0.3 mL) was added HOAc (41.9 mg, 698 μmol) and KOAc (41.1 mg, 419 μmol) and the mixture was stirred at rt for 1 hr. Next, NaBH₃CN (26.3 mg, 419 μmol) was added at 0° C., then the mixture was stirred at it for 1 hr. On completion, the reaction mixture was concentrated under reduced pressure to give a residue. The residue was purified by prep-HPLC (neutral condition) to give the title compound (25.5 mg, 22% yield) as a white solid. LC-MS (ESI⁺) m/z 412.1 (M/2+H)⁺; ¹H NMR (400 MHz, DMSO-d₆) δ=10.89 (s, 1H), 7.77-7.69 (m, 3H), 7.58-7.50 (m, 2H), 7.45-7.37 (m, 2H), 7.29-7.21 (m, 2H), 7.09 (br d, J=8.4 Hz, 2H), 6.20 (br d, J=9.2 Hz, 1H), 4.53-4.43 (m, 1H), 4.40-4.31 (m, 2H), 4.17 (br dd, J=4.8, 12.0 Hz, 1H), 3.95-3.85 (m, 1H), 3.25 (br s, 8H), 3.11-3.01 (m, 4H), 2.98-2.85 (m, 3H), 2.83-2.76 (m, 3H), 2.63 (br s, 6H), 2.55 (br d, J=3.2 Hz, 1H), 2.34-2.26 (m, 1H), 2.01-1.88 (m, 3H), 1.82-1.71 (m, 1H), 1.62-1.35 (m, 1H).

TABLE 3
Compounds synthesized via Method 1, the reductive
amination of the corresponding amines and aldehydes

| | | | LCMS (ESI+) | |
|---|---|---|---|---|
| I-#^{a} | Amine | Aldehyde | m/z (M + H)⁺ | ¹H NMR (400 MHz, DMSO-d₆) δ |
| I-220 | R | K | 442.4^{b} | 12.08 (br s, 1H), 11.09 (s, 1H), 7.71 (dd, J = 1.6, |
| | | | | 10.8 Hz, 1H), 7.51 (br d, J = 8.8 Hz, 2H), 7.41 (dd, |
| | | | | J = 1.2, 17.2 Hz, 1H), 7.09-7.03 (m, 3H), 7.03- |
| | | | | 6.98 (m, 2H), 6.90 (br d, J = 8.0 Hz, 1H), 6.82 (br |
| | | | | s, 1H), 6.65 (br d, J = 3.2 Hz, 1H), 6.19 (td, J = |
| | | | | 1.6, 11.2 Hz, 1H), 5.34 (br dd, J = 5.2, 12.8 Hz, |
| | | | | 1H), 4.46 (br s, 3H), 4.35 (br d, J = 7.2 Hz, 2H), |
| | | | | 4.08 (br s, 2H), 3.88 (br d, J = 12.4 Hz, 1H), 3.33 |
| | | | | (br s, 2H), 3.30 (s, 2H), 3.22 (br s, 3H), 3.08-3.00 |
| | | | | (m, 1H), 2.97-2.85 (m, 4H), 2.80-2.71 (m, 1H), |
| | | | | 2.60 (br s, 1H), 2.55-2.53 (m, 3H), 2.34-2.27 |
| | | | | (m, 3H), 2.08-1.96 (m, 2H), 1.89 (br s, 2H), 1.85- |
| | | | | 1.70 (m, 4H), 1.57-1.34 (m, 2H), 1.23 (br s, 2H) |
| I-242 | F | A | 769.5 | 11.95 (s, 1H), 10.89 (s, 1H), 7.38 (s, 1H), 7.30- |
| | | | | 7.21 (m, 2H), 7.17 (dd, J = 3.2, 8.4 Hz, 1H), 6.92- |
| | | | | 6.79 (m, 1H), 6.66 (s, 1H), 6.60 (d, J = 8.4 Hz, |
| | | | | 1H), 6.48 (d, J = 2.0 Hz, 1H), 6.17-6.04 (m, 1H), |
| | | | | 4.38-4.27 (m, 2H), 4.17 (dd, J = 5.2, 12.4 Hz, |
| | | | | 1H), 3.71 (s, 3H), 3.61 (td, J = 5.6, 12.0 Hz, 2H), |
| | | | | 3.24 (s, 4H), 3.22-2.89 (m, 6H), 2.85-2.72 (m, |
| | | | | 3H), 2.62 (s, 3H), 2.62-2.52 (m, 4H), 2.36 (d, J = |
| | | | | 2.8 Hz, 1H), 2.34-2.21 (m, 2H), 2.08 (s, 2H), 2.06 |
| | | | | (s, 1H), 2.01-1.93 (m, 1H) |
| I-255 | F | B | 753.5 | 11.99-11.88 (m, 1H), 10.84 (s, 1H), 7.32 (t, J = |
| | | | | 8.0 Hz, 1H), 7.20-7.15 (m, 1H), 7.08-6.97 (m, |
| | | | | 2H), 6.92-6.80 (m, 1H), 6.66 (d, J = 1.8 Hz, 1H), |
| | | | | 6.59 (br d, J = 8.0 Hz, 1H), 6.48 (br s, 1H), 6.15- |
| | | | | 6.07 (m, 1H), 4.36-4.28 (m, 2H), 3.87 (dd, J = |
| | | | | 4.8, 12.0 Hz, 1H), 3.71 (s, 3H), 3.65-3.57 (m, |
| | | | | 2H), 3.24 (br s, 5H), 3.19-2.94 (m, 6H), 2.86- |
| | | | | 2.78 (m, 2H), 2.72-2.65 (m, 1H), 2.62 (br s, 3H), |
| | | | | 2.59-2.54 (m, 2H), 2.40-2.31 (m, 2H), 2.28- |
| | | | | 2.17 (m, 2H), 2.09-2.00 (m, 4H) |
| I-263 | O | B | 804.6 | 10.83 (s, 1H), 7.77-7.68 (m, 3H), 7.54 (s, 1H), |
| | | | | 7.52-7.44 (m, 1H), 7.44-7.36 (m, 1H), 7.31 (t, J = |
| | | | | 8.0 Hz, 1H), 7.08 (br d, J = 9.2 Hz, 2H), 7.00 (br |
| | | | | d, J = 8.0 Hz, 1H), 6.24-6.11 (m, 2H), 4.39-4.34 |
| | | | | (m, 3H), 4.29 (br s, 1H), 3.87 (br dd, J = 4.8, 11.6 |
| | | | | Hz, 1H), 3.67-3.56 (m, 2H), 3.26 (br d, J = 10.8 |
| | | | | Hz, 8H), 3.07-2.94 (m, 5H), 2.81 (br t, J = 7.2 |
| | | | | Hz, 2H), 2.70-2.54 (m, 8H), 2.33 (br s, 1H), 2.29- |
| | | | | 2.15 (m, 2H), 2.02 (td, J = 4.0, 8.8 Hz, 1H) |
| I-313 | BP | A | 771.5 | 11.84 (s, 1H), 10.89 (s, 1H), 7.39 (s, 1H), 7.28- |
| | | | | 7.20 (m, 2H), 7.15 (d, J = 8.4 Hz, 1H), 6.88 (dd, J = |
| | | | | 5.6, 10.4 Hz, 1H), 6.66 (s, 1H), 6.60 (d, J = 8.4 |
| | | | | Hz, 1H), 6.46 (t, J = 2.4 Hz, 1H), 4.53-4.41 (m, |
| | | | | 1H), 4.17 (dd, J = 5.2, 12.4 Hz, 1H), 3.85 (d, J = |
| | | | | 13.6 Hz, 1H), 3.71 (s, 3H), 3.24 (s, 4H), 3.20- |
| | | | | 3.01 (m, 6H), 2.98 (d, J = 15.6 Hz, 1H), 2.86-2.75 |
| | | | | (m, 3H), 2.75-2.65 (m, 2H), 2.63 (s, 3H), 2.61- |
| | | | | 2.53 (m, 4H), 2.34-2.23 (m, 1H), 2.03 (d, J = 9.2 |
| | | | | Hz, 3H), 2.00-1.89 (m, 2H), 1.89-1.71 (m, 2H), |
| | | | | 1.67-1.34 (m, 1H) |
| I-336 | AA | S | 859.7 | 12.14 (s, 1H), 11.10 (s, 1H), 7.44 (d, J = 8.4 Hz, |
| | | | | 1H), 7.07 (d, J = 8.8 Hz, 1H), 6.96 (d, J = 12.4 Hz, |
| | | | | 4H), 6.89 (d, J = 6.0 Hz, 1H), 6.53 (s, 1H), 6.11 (s, |
| | | | | 1H), 5.38 (dd, J = 4.8, 12.4 Hz, 1H), 4.44-4.18 |
| | | | | (m, 2H), 3.61 (s, 3H), 3.59 (s, 2H), 3.29 (s, 4H), |
| | | | | 3.23-3.06 (m, 6H), 3.05 (s, 2H), 2.93-2.85 (m, |
| | | | | 1H), 2.72 (s, 2H), 2.67 (s, 3H), 2.61 (s, 3H), 2.39- |
| | | | | 2.24 (m, 2H), 2.07 (d, J = 8.4 Hz, 3H), 2.04- |
| | | | | 1.96 (m, 1H) |
| I-359 | X | S | 869.6 | 12.15-11.99 (m, 1H), 11.10 (s, 1H), 7.35 (br s, |
| | | | | 1H), 6.96 (br dd, J = 5.2, 17.2 Hz, 6H), 6.55 (br s, |
| | | | | 1H), 6.12 (br s, 1H), 5.43-5.31 (m, 1H), 4.35 (br |
| | | | | s, 2H), 3.73-3.57 (m, 5H), 3.29-3.09 (m, 11H), |
| | | | | 3.04 (br d, J = 4.0 Hz, 4H), 2.67 (br s, 8H), 2.38- |
| | | | | 2.21 (m, 2H), 2.04-1.96 (m, 1H), 1.10-0.94 (m, |
| | | | | 6H) |
| I-371 | BA | B | 435.9^{b} | 10.85 (s, 1H), 7.74-7.68 (m, 1H), 7.52 (s, 1H), |
| | | | | 7.45-7.42 (m, 1H), 7.39-7.35 (m, 1H), 7.35- |
| | | | | 7.29 (m, 2H), 7.15-6.94 (m, 4H), 6.81 (s, 1H), |
| | | | | 6.21-6.10 (m, 2H), 4.37-4.32 (m, 3H), 4.27 (br |
| | | | | s, 1H), 3.87 (dd, J = 4.4, 11.8 Hz, 1H), 3.67-3.61 |
| | | | | (m, 1H), 3.57 (br t, J = 5.6 Hz, 1H), 3.27 (br s, |
| | | | | 4H), 3.20 (br s, 3H), 3.00 (br s, 3H), 2.98-2.93 |
| | | | | (m, 2H), 2.84-2.79 (m, 2H), 2.58 (br s, 7H), 2.33 |
| | | | | (br s, 2H), 2.27-2.20 (m, 2H), 2.05-2.00 (m, 1H) |
| I-372 | AZ | B | 834.3 | 10.85 (s, 1H), 7.71 (br d, J = 10.4 Hz, 1H), 7.48 |
| | | | | (s, 1H), 7.44-7.35 (m, 1H), 7.34-7.24 (m, 3H), |
| | | | | 7.12-6.95 (m, 2H), 6.70-6.58 (m, 2H), 6.23- |
| | | | | 6.07 (m, 2H), 4.41-4.30 (m, 3H), 4.26 (br s, 1H), |
| | | | | 3.87 (br dd, J = 4.0, 11.6 Hz, 1H), 3.72 (br d, J = |
| | | | | 4.4 Hz, 3H), 3.63 (br s, 1H), 3.57 (br s, 1H), 3.26 |
| | | | | (br s, 6H), 3.21 (br s, 1H), 2.99 (br d, J = 6.0 Hz, |
| | | | | 5H), 2.81 (br t, J = 7.2 Hz, 2H), 2.71-2.56 (m, |
| | | | | 8H), 2.32 (br s, 1H), 2.25 (br s, 2H), 2.02 (br dd, J = |
| | | | | 3.6, 4.8 Hz, 1H) |
| I-383 | BB | B | 834.8 | 2.00-2.06 (m, 1 H) 2.19-2.29 (m, 2 H) 2.35 (br |
| | | | | dd, J = 3.6, 1.6 Hz, 2 H) 2.55-2.75 (m, 9 H) 2.80- |
| | | | | 2.85 (m, 2 H) 3.02-3.13 (m, 6 H) 3.25 (br s, 4 |
| | | | | H) 3.58-3.61 (m, 1 H) 3.65 (br t, J = 5.6 Hz, 1 H) |
| | | | | 3.70-3.72 (m, 3 H) 3.88 (dd, J = 11.2, 4.75 Hz, 1 |
| | | | | H) 4.30-4.37 (m, 2 H) 4.58-4.66 (m, 2 H) 6.08- |
| | | | | 6.14 (m, 1 H) 6.47-6.50 (m, 1 H) 6.60 (dd, J = |
| | | | | 8.4, 1.8 Hz, 1 H) 6.67 (s, 1 H) 6.82-6.90 (m, 1 H) |
| | | | | 7.01 (d, J = 8.0 Hz, 1 H) 7.06 (d, J = 11.2 Hz, 1 |
| | | | | H) 7.17 (d, J = 8.4 Hz, 1 H) 7.31-7.36 (m, 1 H) |
| | | | | 7.67-7.71 (m, 1 H) 8.09-8.13 (m, 1 H) 8.34 (s, |
| | | | | 1 H) 10.85 (s, 1 H) 11.95 (br d, J - 8.0 Hz, 1 H) |
| I-429 | BZ | CA | 897.5 | 11.92 (s, 1H), 11.12-11.06 (m, 1H), 8.15 (s, 1H), |
| | | | | 7.77-7.66 (m, 1H), 7.51 (d, J = 8.8 Hz, 2H), 7.41 |
| | | | | (dd, J = 1.6, 16.4 Hz, 1H), 7.05 (d, J = 8.8 Hz, 3H), |
| | | | | 6.99-6.95 (m, 3H), 6.93-6.90 (m, 1H), 6.20 (td, |
| | | | | J = 2.0, 10.4 Hz, 1H), 5.36 (dd, J = 5.6, 12.4 Hz, |
| | | | | 1H), 4.53-4.45 (m, 1H), 4.35 (q, J = 6.4 Hz, 2H), |
| | | | | 3.89 (d, J = 12.4 Hz, 1H), 3.59 (s, 3H), 3.26 (d, J = |
| | | | | 12.8 Hz, 1H), 3.19 (s, 3H), 3.13 (s, 1H), 3.04 (d, |
| | | | | J = 1.6 Hz, 4H), 2.93 (dt, J = 8.0, 15.6 Hz, 6H), |
| | | | | 2.85-2.77 (m, 1H), 2.75-2.67 (m, 1H), 2.62 (d, |
| | | | | J = 4.4 Hz, 1H), 2.54 (d, J = 2.4 Hz, 4H), 2.44 (t, |
| | | | | J = 6.8 Hz, 2H), 2.01-1.95 (m, 1H), 1.92 (d, J = |
| | | | | 15.6 Hz, 2H), 1.81 (dd, J = 7.6, 14.4 Hz, 2H), 1.74 |
| | | | | (s, 1H), 1.56-1.40 (m, 1H), 0.75 (t, J = 5.6 Hz, |
| | | | | 2H), 0.61 (s, 2H) |
| I-433 | CE | S | 902.2 | 11.17-11.03 (m, 1H), 8.05 (s, 1H), 7.67 (d, J = |
| | | | | 3.2 Hz, 1H), 7.55 (d, J = 3.6 Hz, 1H), 7.44-7.40 |
| | | | | (m, 1H), 7.35-7.27 (m, 1H), 7.00 (br d, J = 5.2 |
| | | | | Hz, 2H), 6.97-6.95 (m, 1H), 6.91-6.86 (m, 1H), |
| | | | | 6.72-6.69 (m, 1H), 6.67-6.61 (m, 1H), 5.39 (br |
| | | | | dd, J = 5.6, 13.2 Hz, 1H), 4.46 (br s, 3H), 3.74 (s, |
| | | | | 3H), 3.63 (s, 3H), 3.26-3.22 (m, 4H), 3.18-3.12 |
| | | | | (m, 3H), 3.02 (s, 6H), 2.95-2.87 (m, 4H), 2.70- |
| | | | | 2.65 (m, 8H), 2.46 (br d, J = 5.6 Hz, 2H), 2.35- |
| | | | | 2.33 (m, 1H), 2.04-1.99 (m, 1H) |
| I-435 | CF | S | 883.4 | 12.05-11.94 (m, 1H), 11.15-11.05 (m, 1H), 7.73- |
| | | | | 7.69 (m, 1H), 7.54-7.50 (m, 2H), 7.44-7.39 (m, |
| | | | | 1H), 7.09-7.05 (m, 2H), 7.00-6.93 (m, 5H), 6.20 |
| | | | | (td, J = 2.0, 10.8 Hz, 1H), 5.38 (br dd, J = 5.2, 12.4 |
| | | | | Hz, 1H), 4.48 (br d, J = 12.8 Hz, 1H), 4.35 (q, J = |
| | | | | 7.6 Hz, 2H), 3.88 (br d, J = 12.8 Hz, 1H), 3.75 (br |
| | | | | s, 2H), 3.62 (s, 3H), 3.54 (br t, J = 6.4 Hz, 2H), |
| | | | | 3.24 (br s, 4H), 3.16-3.10 (m, 2H), 3.08-3.02 |
| | | | | (m, 1H), 2.98-2.85 (m, 4H), 2.77 (br s, 1H), 2.70- |
| | | | | 2.62 (m, 8H), 2.02-1.97 (m, 1H), 1.94-1.84 (m, |
| | | | | 6H), 1.79-1.73 (m, 1H), 1.52-1.38 (m, 1H) |
| I-463 | CK | S | 892.4 | 11.11 (s, 1H), 7.71 (dd, J = 2.0, 8.0 Hz, 1H), 7.52 |
| | | | | (d, J = 1.6 Hz, 1H), 7.47-7.32 (m, 3H), 7.14 (s, |
| | | | | 1H), 7.05 (d, J = 8.4 Hz, 1H), 7.02-6.96 (m, 2H), |
| | | | | 6.96-6.92 (m, 1H), 6.19 (td, J = 1.8, 8.4 Hz, 1H), |
| | | | | 5.38 (dd, J = 5.6, 12.4 Hz, 1H), 4.47 (d, J = 12.0 |
| | | | | Hz, 1H), 4.39-4.29 (m, 2H), 3.88 (t, J = 11.2 Hz, |
| | | | | 1H), 3.61 (s, 3H), 3.31-3.24 (m, 3H), 3.19 (s, |
| | | | | 3H), 3.15 (d, J = 7.2 Hz, 3H), 3.07-2.97 (m, 4H), |
| | | | | 2.97-2.83 (m, 4H), 2.83-2.52 (m, 9H), 2.08- |
| | | | | 1.96 (m, 1H), 1.96-1.80 (m, 2H), 1.79-1.68 (m, |
| | | | | 1H), 1.56-1.36 (m, 1H) |
| I-468 | CN | S | 868.4 | 11.10 (s, 1H), 10.47 (s, 1H), 8.12 (d, J = 2.8 Hz, |
| | | | | 1H), 7.70 (d, J = 2.8 Hz, 1H), 7.52-7.47 (m, 1H), |
| | | | | 7.15 (d, J = 8.0 Hz, 1H), 7.03-6.88 (m, 5H), 6.73- |
| | | | | 6.66 (m, 2H), 6.62 (d, J = 1.6 Hz, 2H), 5.38 (dd, |
| | | | | J = 5.2, 12.8 Hz, 1H), 4.66-4.62 (m, 2H), 4.36 (d, |
| | | | | J = 10.8 Hz, 2H), 3.73 (s, 3H), 3.62 (s, 3H), 3.58- |
| | | | | 3.55 (m, 1H), 3.28-3.26 (m, 4H), 3.17-3.10 (m, |
| | | | | 4H), 3.09 (s, 1H), 3.00 (s, 6H), 2.91-2.86 (m, |
| | | | | 1H), 2.68 (d, J = 5.2 Hz, 4H), 2.64 (s, 2H), 2.34- |
| | | | | 2.28 (m, 2H), 2.23 (s, 1H), 2.04-1.98 (m, 1H) |
| I-488 | CR | S | 838.6 | 11.10 (s, 1H), 10.79 (s, 1H), 8.12 (d, J = 2.8 Hz, |
| | | | | 1H), 7.72-7.67 (m, 1H), 7.53-7.48 (m, 3H), 7.09 |
| | | | | (d, J = 8.8 Hz, 2H), 7.03 (s, 1H), 6.99 (d, J = 5.2 |
| | | | | Hz, 2H), 6.96-6.93 (m, 1H), 6.74-6.57 (m, 2H), |
| | | | | 5.38 (dd, J = 5.2, 12.8 Hz, 1H), 4.64 (t, J = 6.8 Hz, |
| | | | | 2H), 4.42-4.35 (m, 2H), 3.62 (s, 3H), 3.59-3.56 |
| | | | | (m, 1H), 3.26 (s, 4H), 3.18-3.07 (m, 5H), 3.00 (s, |
| | | | | 6H), 2.92-2.85 (m, 1H), 2.71-2.63 (m, 8H), 2.33- |
| | | | | 2.24 (m, 2H), 2.02-1.97 (m, 1H) |
| I-498 | CU | S | 861.4 | 12.04 (s, 1H), 11.10 (s, 1H), 7.43 (dd, J = 2.0, 8.4 |
| | | | | Hz, 1H), 7.09 (d, J = 8.4 Hz, 1H), 7.04-6.77 (m, |
| | | | | 5H), 6.52 (s, 1H), 5.38 (dd, J = 5.6, 12.8 Hz, 1H), |
| | | | | 4.50-4.42 (m, 1H), 3.85 (d, J = 12.8 Hz, 1H), 3.62 |
| | | | | (s, 3H), 3.25-2.96 (m, 12H), 2.95-2.81 (m, 2H), |
| | | | | 2.77-2.56 (m, 10H), 2.03 (d, J = 9.6 Hz, 3H), 1.99- |
| | | | | 1.71 (m, 4H), 1.62-1.41 (m, 1H) |
| I-499 | CV | B | 888.3 | 12.14 (d, J = 6.8 Hz, 1H), 10.85 (s, 1H), 8.13- |
| | | | | 8.08 (m, 1H), 7.72-7.65 (m, 1H), 7.43 (d, J = 8.4 |
| | | | | Hz, 1H), 7.32 (t, J = 8.0 Hz, 1H), 7.09-7.03 (m, |
| | | | | 2H), 7.00 (d, J = 8.0 Hz, 1H), 6.97-6.87 (m, 2H), |
| | | | | 6.53 (s, 1H), 6.19-6.04 (m, 1H), 4.62 (q, J = 7.2 |
| | | | | Hz, 2H), 4.42-4.24 (m, 2H), 3.87 (dd, J = 4.8, |
| | | | | 12.0 Hz, 1H), 3.69-3.55 (m, 2H), 3.30-3.13 (m, |
| | | | | 8H), 3.12-2.99 (m, 5H), 2.86-2.75 (m, 2H), 2.74- |
| | | | | 2.55 (m, 7H), 2.34 (s, 1H), 2.31-2.14 (m, 2H), |
| | | | | 2.06-1.96 (m, 1H) |
| I-509 | CX | S | 787.2 | 11.09 (s, 1H), 10.51-10.43 (m, 1H), 7.50 (s, 1H), |
| | | | | 7.16 (br d, J = 8.4 Hz, 1H), 7.01-6.93 (m, 4H), |
| | | | | 6.67 (br s, 2H), 6.65-6.50 (m, 2H), 5.42-5.32 |
| | | | | (m, 1H), 4.36 (br s, 2H), 3.74 (d, J = 4.0 Hz, 3H), |
| | | | | 3.62 (s, 3H), 3.60-3.54 (m, 2H), 3.29 (br s, 3H), |
| | | | | 3.17-3.11 (m, 2H), 3.00 (s, 6H), 2.94-2.85 (m, |
| | | | | 1H), 2.78-2.59 (m, 9H), 2.35-2.20 (m, 2H), 2.10 |
| | | | | (d, J = 18.0 Hz, 3H), 2.04-1.97 (m, 1H) |
| I-531 | BE | S | 887.3 | 12.15 (s, 1H), 11.10 (s, 1H), 7.44 (d, J = 8.4 Hz, |
| | | | | 1H), 7.08 (d, J = 9.2 Hz, 1H), 6.99 (d, J = 5.2 Hz, |
| | | | | 3H), 6.95-6.93 (m, 1H), 6.89 (d, J = 6.0 Hz, 1H), |
| | | | | 6.54 (s, 1H), 6.11 (s, 1H), 5.38 (dd, J = 5.2, 12.8 |
| | | | | Hz, 1H), 4.44-4.32 (m, 2H), 3.66 (q, J = 6.0 Hz, |
| | | | | 2H), 3.61 (s, 3H), 3.29 (s, 6H), 3.18-3.10 (m, |
| | | | | 4H), 3.05 (dd, J = 3.6, 10.8 Hz, 2H), 2.95-2.84 |
| | | | | (m, 2H), 2.79-2.70 (m, 1H), 2.66 (d, J = 4.4 Hz, |
| | | | | 7H), 2.37 (d, J = 1.6 Hz, 1H), 2.26 (d, J = 0.8 Hz, |
| | | | | 1H), 2.06-1.95 (m, 1H), 1.06-0.97 (m, 6H) |
| I-536 | AA | AB | 843.3 | 13.23-11.37 (m, 1H), 11.30-10.01 (m, 1H), 7.50- |
| | | | | 7.36 (m, 2H), 7.29 (br t, J = 7.6 Hz, 1H), 7.09 (br |
| | | | | d, J = 8.0 Hz, 1H), 7.02-6.84 (m, 3H), 6.55 (br s, |
| | | | | 1H), 6.12 (br s, 1H), 5.73 (br dd, J = 4.8, 11.4 Hz, |
| | | | | 1H), 4.40-4.26 (m, 2H), 3.69-3.55 (m, 2H), 3.32- |
| | | | | 3.28 (m, 4H), 3.25-2.98 (m, 8H), 2.90-2.81 (m, |
| | | | | 1H), 2.77-2.62 (m, 11H), 2.38 (br s, 1H), 2.34- |
| | | | | 2.16 (m, 2H), 2.08 (br d, J = 8.8 Hz, 3H) |
| I-540 | CZ | S | 855.4 | 11.12-11.06 (m, 1H), 8.19-8.04 (m, 1H), 7.68 |
| | | | | (d, J = 2.0 Hz, 1H), 7.65-7.46 (m, 2H), 7.42- |
| | | | | 7.31 (m, 2H), 7.30-7.20 (m, 1H), 7.11-7.02 (m, |
| | | | | 1H), 6.95 (br d, J = 13.2 Hz, 4H), 6.10 (s, 1H), |
| | | | | 5.41-5.33 (m, 1H), 4.55 (br s, 2H), 4.40-4.33 |
| | | | | (m, 2H), 3.67-3.62 (m, 1H), 3.61-3.57 (m, 4H), |
| | | | | 3.28-3.21 (m, 3H), 3.17-3.07 (m, 2H), 3.06- |
| | | | | 2.93 (m, 9H), 2.90-2.84 (m, 1H), 2.80-2.73 (m, |
| | | | | 1H), 2.73-2.62 (m, 6H), 2.60 (br s, 1H), 2.43 (br |
| | | | | d, J = 3.6 Hz, 1H), 2.41-2.31 (m, 1H), 2.02-1.94 |
| | | | | (m, 1H) |
| I-563 | CZ | DH | 841.2 | 11.15 (s, 1H), 8.16-8.08 (m, 1H), 7.72-7.61 (m, |
| | | | | 1H), 7.58 (br s, 2H), 7.36 (s, 1H), 7.34-7.12 (m, |
| | | | | 2H), 7.10-6.98 (m, 2H), 6.97-6.85 (m, 3H), 6.25- |
| | | | | 6.09 (m, 1H), 5.43-5.24 (m, 1H), 4.57-4.42 (m, |
| | | | | 2H), 4.35-4.23 (m, 2H), 3.76-3.64 (m, 5H), 3.64- |
| | | | | 3.54 (m, 2H), 3.27-3.20 (m, 4H), 3.05-2.98 (m, |
| | | | | 5H), 2.94-2.85 (m, 2H), 2.63 (br s, 3H), 2.58 (br |
| | | | | d, J = 2.8 Hz, 2H), 2.41 (br s, 2H), 2.04-1.94 (m, |
| | | | | 1H), 1.40-1.23 (m, 3H) |
| I-571 | DI | DJ | 832.9 | 11.93 (d, J = 9.2 Hz, 1H), 10.85 (s, 1H), 7.74- |
| | | | | 7.68 (m, 1H), 7.44-7.35 (m, 1H), 7.25-7.19 (m, |
| | | | | 1H), 7.19-7.06 (m, 3H), 6.88-6.81 (m, 1H), 6.66 |
| | | | | (s, 1H), 6.62-6.57 (m, 1H), 6.48 (s, 1H), 6.22- |
| | | | | 6.15 (m, 1H), 6.12-6.06 (m, 1H), 4.39-4.26 (m, |
| | | | | 4H), 4.01 (dd, J = 4.8, 12.4 Hz, 1H), 3.73-3.68 |
| | | | | (m, 3H), 3.66-3.55 (m, 2H), 3.24 (s, 5H), 3.19- |
| | | | | 3.04 (m, 5H), 3.03-2.92 (m, 3H), 2.83-2.78 (m, |
| | | | | 2H), 2.76-2.68 (m, 1H), 2.65-2.58 (m, 6H), 2.34- |
| | | | | 2.24 (m, 2H), 2.22-2.12 (m, 1H), 2.04-1.94 (m, |
| | | | | 1H), 1.25-1.22 (m, 1H) |
| I-604 | DM | B | 833.4 | 12.00 (d, J = 8.8 Hz, 1H), 10.85 (s, 1H), 8.34 (s, |
| | | | | 1H), 8.14-8.04 (m, 1H), 7.72-7.64 (m, 1H), 7.34- |
| | | | | 7.21 (m, 2H), 7.05 (d, J = 11.6 Hz, 1H), 7.02- |
| | | | | 6.98 (m, 1H), 6.97-6.80 (m, 2H), 6.47 (s, 1H), |
| | | | | 6.12 (d, J = 12.8 Hz, 1H), 4.69-4.56 (m, 2H), 4.46- |
| | | | | 4.24 (m, 2H), 3.87-3.89 (m, 1H), 3.76-3.70 (m, |
| | | | | 3H), 3.65-3.58 (m, 2H), 3.26-3.13 (m, 4H), 3.12- |
| | | | | 3.02 (m, 6H), 2.80-2.83 (m, 2H), 2.73-2.64 (m, |
| | | | | 1H), 2.61-2.53 (m, 4H), 2.33 (s, 1H), 2.25-2.28 |
| | | | | (m, 2H), 2.16-2.05 (m, 2H), 2.02-2.06 (m, 1H), |
| | | | | 1.91-1.66 (m, 4H) |
| I-617 | AA | DR | 842.3 | 12.19-12.10 (m, 1H), 10.90 (s, 1H), 7.62-7.54 |
| | | | | (m, 1H), 7.49-7.40 (m, 1H), 7.26-7.19 (m, 1H), |
| | | | | 7.12-7.03 (m, 2H), 7.00 (br s, 1H), 6.97-6.89 |
| | | | | (m, 1H), 6.56-6.48 (m, 1H), 6.12 (br s, 1H), 4.39- |
| | | | | 4.31 (m, 3H), 4.26 (s, 3H), 3.67-3.53 (m, 3H), |
| | | | | 3.22-2.96 (m, 9H), 2.84-2.59 (m, 8H), 2.41- |
| | | | | 2.31 (m, 3H), 2.27 (br d, J = 2.8 Hz, 1H), 2.18 (br |
| | | | | dd, J = 5.6, 13.2 Hz, 1H), 2.08 (br d, J = 9.2 Hz, |
| | | | | 3H) |
| I-635 | DV | B | 848.3 | 12.05 (d, J = 7.6 Hz, 1H), 10.85 (s, 1H), 8.14- |
| | | | | 8.07 (m, 1H), 7.72-7.63 (m, 1H), 7.39-7.30 (m, |
| | | | | 2H), 7.15 (dd, J = 1.6, 3.6 Hz, 1H), 7.10-7.05 (m, |
| | | | | 1H), 7.02 (ddd, J = 1.6, 4.0, 8.0 Hz, 2H), 6.98- |
| | | | | 6.89 (m, 1H), 6.49 (s, 1H), 6.17-6.08 (m, 1H), |
| | | | | 4.67-4.57 (m, 2H), 4.41-4.27 (m, 2H), 3.88 (dd, |
| | | | | J = 4.8, 12.0 Hz, 1H), 3.79-3.73 (m, 2H), 3.73- |
| | | | | 3.68 (m, 3H), 3.68-3.55 (m, 3H), 3.22-2.97 (m, |
| | | | | 10H), 2.93-2.80 (m, 4H), 2.73-2.62 (m, 3H), |
| | | | | 2.37-2.20 (m, 3H), 2.08-1.97 (m, 1H) |
| I-637 | DX | S | 809.3 | 12.25-11.73 (m, 1H), 11.10 (s, 1H), 7.32 (d, J = |
| | | | | 8.8 Hz, 1H), 7.11 (s, 1H), 7.06-6.99 (m, 2H), 6.98- |
| | | | | 6.90 (m, 2H), 6.86 (d, J = 6.4 Hz, 1H), 6.46 (s, |
| | | | | 1H), 6.24-6.03 (m, 1H), 5.38-5.39 (m, 1H), 4.33 |
| | | | | (d, J = 7.6 Hz, 2H), 3.61 (s, 3H), 3.60-3.58 (m, |
| | | | | 1H), 3.28 (d, J = 1.6 Hz, 4H), 3.24-3.09 (m, 6H), |
| | | | | 3.08-3.02 (m, 2H), 3.01-2.83 (m, 2H), 2.80- |
| | | | | 2.69 (m, 2H), 2.66 (d, J = 3.6 Hz, 3H), 2.61 (s, |
| | | | | 2H), 2.44-2.31 (m, 2H), 2.26 (s, 1H), 2.07 (d, J = |
| | | | | 6.4 Hz, 3H), 2.04-1.94 (m, 1H) |
| I-640 | EI | AB | 856.3 | 11.07 (s, 1H), 7.74-7.67 (m, 2H), 7.49 (d, J = 8.8 |
| | | | | Hz, 2H), 7.45-7.40 (m, 1H), 7.39-7.27 (m, 3H), |
| | | | | 7.10 (br d, J = 8.8 Hz, 2H), 6.97 (d, J = 6.8 Hz, |
| | | | | 1H), 6.27 (br s, 1H), 6.23-6.16 (m, 1H), 5.76- |
| | | | | 5.70 (m, 1H), 4.40-4.32 (m, 4H), 3.67-3.58 (m, |
| | | | | 2H), 3.30-3.27 (m, 6H), 3.24-3.18 (m, 4H), 3.09- |
| | | | | 3.02 (m, 2H), 3.02-2.93 (m, 3H), 2.88-2.82 (m, |
| | | | | 1H), 2.74 (br d, J = 2.8 Hz, 1H), 2.70 (br s, 4H), |
| | | | | 2.67 (s, 4H), 2.36 (br dd, J = 5.6, 9.2 Hz, 2H), 2.31- |
| | | | | 2.20 (m, 2H) |
| I-643 | BE | DR | 871.4 | 12.15 (brs, 1H), 10.90 (s, 1H), 7.57 (d, J = 8.0 Hz, |
| | | | | 1H), 7.45 (br d, J = 8.4 Hz, 1H), 7.23 (d, J = 6.8 |
| | | | | Hz, 1H), 7.10-7.02 (m, 2H), 6.99-6.89 (m, 2H), |
| | | | | 6.54 (br s, 1H), 6.12 (br s, 1H), 4.37 (br dd, J = |
| | | | | 4.8, 9.6 Hz, 3H), 4.25 (s, 3H), 3.67 (br d, J = 5.6 |
| | | | | Hz, 3H), 3.30 (br s, 4H), 3.22-3.01 (m, 6H), 2.94 |
| | | | | (br dd, J = 6.4, 10.4 Hz, 1H), 2.75-2.60 (m, 9H), |
| | | | | 2.39-2.24 (m, 3H), 2.22-2.14 (m, 1H), 1.06- |
| | | | | 0.99 (m, 6H) |
| I-659 | FZ | FY | 834.7 | 12.05-11.94 (m, 1H), 10.81 (s, 1H), 8.14-8.06 |
| | | | | (m, 1H), 7.73-7.63 (m, 1H), 7.24 (d, J = 7.2 Hz, |
| | | | | 1H), 7.07-7.01 (m, 2H), 7.01-6.94 (m, 2H), 6.92 |
| | | | | (d, J = 7.2 Hz, 2H), 6.46 (s, 1H), 6.15-6.08 (m, |
| | | | | 1H), 4.66-4.58 (m, 2H), 4.39-4.28 (m, 2H), 3.80 |
| | | | | (dd, J = 4.0, 11.2 Hz, 1H), 3.72 (s, 3H), 3.66-3.58 |
| | | | | (m, 2H), 3.23-2.97 (m, 13H), 2.87-2.80 (m, 2H), |
| | | | | 2.65 (s, 7H), 2.36-2.26 (m, 2H), 2.23-2.14 (m, |
| | | | | 1H), 2.04-1.96 (m, 1H) |
| I-662 | EL | S | 839.2 | 1.53-1.67 (m, 3 H) 1.98-2.10 (m, 4 H) 2.21- |
| | | | | 2.40 (m, 3 H) 2.57-2.76 (m, 9 H) 2.87-3.18 (m, |
| | | | | 10 H) 3.56-3.64 (m, 5 H) 4.22-4.37 (m, 2 H) |
| | | | | 5.38 (br dd, J = 12.0, 4.82 Hz, 1 H) 6.10 (br d, J = |
| | | | | 9.6 Hz, 1 H) 6.28 (br s, 1 H) 6.72-6.85 (m, 1 H) |
| | | | | 6.91-7.02 (m, 3 H) 7.06-7.20 (m, 3 H) 11.10 (br |
| | | | | s, 1 H) 12.04-12.14 (m, 1 H) |
| I-670 | EO | DR | 805.8 | 12.15-12.07 (m, 1H), 10.91-10.85 (m, 1H), 7.59- |
| | | | | 7.54 (m, 1H), 7.43-7.37 (m, 1H), 7.24-7.20 (m, |
| | | | | 1H), 7.07-7.02 (m, 1H), 6.97-6.87 (m, 3H), 6.63- |
| | | | | 6.58 (m, 1H), 6.15-6.10 (m, 1H), 4.40-4.34 (m, |
| | | | | 3H), 4.25 (s, 3H), 3.69-3.63 (m, 2H), 3.27-3.24 |
| | | | | (m, 3H), 3.19-2.91 (m, 8H), 2.73-2.61 (m, 9H), |
| | | | | 2.40-2.30 (m, 3H), 2.30-2.23 (m, 1H), 2.20- |
| | | | | 2.14 (m, 1H), 1.04 (d, J = 6.8 Hz, 6H) |
| I-671 | EO | AB | 805.7 | 12.13 (br s, 1H), 11.06 (s, 1H), 7.52-7.35 (m, |
| | | | | 2H), 7.29 (t, J = 7.8 Hz, 1H), 7.06-6.81 (m, 4H), |
| | | | | 6.62 (br d, J = 2.0 Hz, 1H), 6.13 (br s, 1H), 5.73 |
| | | | | (dd, J = 5.2, 11.6 Hz, 1H), 4.50-4.29 (m, 2H), |
| | | | | 3.67 (br d, J = 5.6 Hz, 2H), 3.40 (br s, 4H), 3.23- |
| | | | | 2.92 (m, 3H), 2.88-2.80 (m, 1H), 2.75-2.65 (m, |
| | | | | 10H), 2.55 (br s, 1H), 2.41-2.20 (m, 3H), 1.18- |
| | | | | 0.92 (m, 6H) |
| I-672 | EP | AB | 839.5 | 12.19 (br d, J = 1.6 Hz, 1H), 11.05 (br s, 1H), 7.40 |
| | | | | (d, J = 8.4 Hz, 1H), 7.35-7.26 (m, 2H), 7.19 (d, J = |
| | | | | 8.0 Hz, 1H), 7.00-6.89 (m, 2H), 6.52 (br d, J = |
| | | | | 2.0 Hz, 1H), 6.15 (br s, 1H), 5.78-5.67 (m, 1H), |
| | | | | 4.44-4.34 (m, 2H), 3.67 (br d, J = 5.6 Hz, 2H), |
| | | | | 3.27-3.22 (m, 2H), 3.18 (br s, 8H), 3.08-3.02 |
| | | | | (m, 2H), 3.00-2.90 (m, 2H), 2.88-2.82 (m, 1H), |
| | | | | 2.76-2.69 (m, 6H), 2.67 (s, 3H), 2.41-2.33 (m, |
| | | | | 2H), 2.29-2.19 (m, 2H), 1.08-0.99 (m, 6H) |
| I-813 | EI | DR | 856.5 | 10.88 (s, 1H), 7.73-7.67 (m, 2H), 7.56 (d, J = 8.0 |
| | | | | Hz, 1H), 7.48 (d, J = 8.0 Hz, 2H), 7.43-7.29 (m, |
| | | | | 2H), 7.22 (d, J = 6.4 Hz, 1H), 7.11-7.02 (m, 3H), |
| | | | | 6.26 (d, J = 0.8 Hz, 1H), 6.19 (br d, J = 13.6 Hz, |
| | | | | 1H), 4.37 (br d, J = 4.4 Hz, 3H), 4.35-4.32 (m, |
| | | | | 2H), 4.25 (s, 3H), 3.67-3.62 (m, 1H), 3.59 (br t, |
| | | | | J = 5.2 Hz, 1H), 3.28 (br s, 8H), 3.16 (br dd, J = |
| | | | | 4.8, 8.8 Hz, 5H), 3.00-2.96 (m, 2H), 2.70 (br s, |
| | | | | 6H), 2.64-2.62 (m, 1H), 2.34 (br d, J = 3.6 Hz, |
| | | | | 2H), 2.31-2.26 (m, 1H), 2.21-2.14 (m, 1H) |
| I-828 | ES | S | 839.3 | 12.24-12.15 (m, 1H), 11.12-11.04 (m, 1H), 7.41- |
| | | | | 7.33 (m, 1H), 7.06-6.92 (m, 5H), 6.64 (br s, 1H), |
| | | | | 6.17-6.12 (m, 1H), 5.45-5.29 (m, 1H), 4.41- |
| | | | | 4.32 (m, 2H), 3.69-3.63 (m, 2H), 3.61 (s, 3H), |
| | | | | 3.15 (br s, 8H), 3.06-3.04 (m, 1H), 2.97-2.93 |
| | | | | (m, 1H), 2.77-2.66 (m, 6H), 2.66-2.62 (m, 2H), |
| | | | | 2.57-2.52 (m, 2H), 2.38 (br d, J = 2.4 Hz, 2H), |
| | | | | 2.34-2.24 (m, 2H), 2.03-1.96 (m, 1H), 1.06- |
| | | | | 0.98 (m, 6H) |
| I-846 | DX | DR | 792.3 | 12.13 (s, 1H), 10.95-10.86 (m, 1H), 7.57 (d, J = |
| | | | | 8.0 Hz, 1H), 7.33 (d, J = 8.4 Hz, 1H), 7.23 (d, J = |
| | | | | 6.8 Hz, 1H), 7.11 (s, 1H), 7.08-7.00 (m, 2H), 6.96- |
| | | | | 6.86 (m, 1H), 6.47 (s, 1H), 6.14 (s, 1H), 4.38- |
| | | | | 4.32 (m, 3H), 4.26 (s, 3H), 3.61 (dd, J = 5.6, 10.8 |
| | | | | Hz, 3H), 3.29 (s, 5H), 3.19-3.00 (m, 6H), 2.75- |
| | | | | 2.63 (m, 8H), 2.38 (d, J = 1.6 Hz, 2H), 2.31-2.23 |
| | | | | (m, 1H), 2.18 (dd, J = 4.8, 13.2 Hz, 1H), 2.14- |
| | | | | 2.04 (m, 3H) |
| I-849 | EE | S | 811.1 | 12.44-11.98 (m, 1H), 11.24-10.89 (m, 1H), 7.44 |
| | | | | (s, 1H), 7.35-7.25 (m, 2H), 7.22-7.14 (m, 1H), |
| | | | | 7.03-6.87 (m, 2H), 6.57-6.47 (m, 1H), 6.22- |
| | | | | 6.09 (m, 1H), 5.79-5.62 (m, 1H), 4.43-4.24 (m, |
| | | | | 2H), 3.66-3.58 (m, 2H), 3.23-3.12 (m, 10H), |
| | | | | 3.07-3.00 (m, 2H), 2.77-2.67 (m, 10H), 2.44- |
| | | | | 2.17 (m, 5H), 2.12-2.04 (m, 3H) |
| I-850 | EE | DR | 811.2 | 12.19 (br s, 1H), 10.89 (s, 1H), 7.57 (d, J = 7.6 Hz, |
| | | | | 1H), 7.31 (dd, J = 2.0, 13.2 Hz, 1H), 7.20 (dd, J = |
| | | | | 7.6, 15.6 Hz, 2H), 7.08-6.89 (m, 2H), 6.50 (s, |
| | | | | 1H), 6.20-6.10 (m, 1H), 4.39-4.30 (m, 3H), 4.25 |
| | | | | (s, 3H), 3.64-3.57 (m, 2H), 3.26 (d, J = 9.6 Hz, |
| | | | | 5H), 3.16 (s, 5H), 3.05 (dd, J = 1.6, 5.2 Hz, 2H), |
| | | | | 2.72 (d, J = 4.0 Hz, 5H), 2.68-2.61 (m, 2H), 2.54 |
| | | | | (s, 1H), 2.39-2.32 (m, 2H), 2.26 (s, 1H), 2.21- |
| | | | | 2.14 (m, 1H), 2.07 (d, J = 6.0 Hz, 3H) |
| I-858 | GC | S | 837.1 | 12.26-11.94 (m, 1H), 11.25-10.91 (m, 1H), 7.32 |
| | | | | (d, J = 8.4 Hz, 1H), 7.10 (s, 1H), 7.05-6.96 (m, |
| | | | | 3H), 6.95-6.84 (m, 2H), 6.47 (s, 1H), 6.13 (s, |
| | | | | 1H), 5.45-5.30 (m, 1H), 4.41-4.32 (m, 2H), 3.68- |
| | | | | 3.59 (m, 5H), 3.27 (s, 4H), 3.16-3.04 (m, 6H), |
| | | | | 3.01-2.83 (m, 3H), 2.71-2.59 (m, 8H), 2.43- |
| | | | | 2.31 (m, 2H), 2.30-2.17 (m, 1H), 2.06-1.96 (m, |
| | | | | 1H), 1.06-0.97 (m, 6H) |
| I-948 | GR | DR | 891.3 | 12.18 (s, 1H), 10.89 (s, 1H), 7.77-7.65 (m, 1H), |
| | | | | 7.57 (d, J = 8.0 Hz, 1H), 7.46-7.36 (m, 1H), 7.34- |
| | | | | 7.27 (m, 1H), 7.25-7.16 (m, 2H), 7.09-7.01 (m, |
| | | | | 1H), 7.00-6.86 (m, 1H), 6.55-6.46 (m, 1H), 6.23- |
| | | | | 6.09 (m, 2H), 4.39-4.34 (m, 4H), 4.29 (s, 1H), |
| | | | | 4.25 (s, 3H), 3.66-3.62 (m, 1H), 3.59-3.57 (m, |
| | | | | 1H), 3.17 (s, 6H), 3.10-3.02 (m, 3H), 3.01-2.94 |
| | | | | (m, 3H), 2.73 (d, J = 2.8 Hz, 4H), 2.70-2.60 (m, |
| | | | | 4H), 2.37 (d, J = 4.8 Hz, 2H), 2.35-2.31 (m, 2H), |
| | | | | 2.29-2.23 (m, 1H), 2.26 (s, 1H) |
| I-978 | GW | DR | 819.4 | 12.15-12.08 (m, 1H), 10.88 (s, 1H), 7.56 (d, J = |
| | | | | 8.0 Hz, 1H), 7.40 (s, 1H), 7.22 (d, J = 6.8 Hz, 1H), |
| | | | | 7.06 (d, J = 8.0 Hz, 1H), 6.90 (d, J = 14.0 Hz, 3H), |
| | | | | 6.61 (d, J = 2.4 Hz, 1H), 6.12 (s, 1H), 4.36 (dd, J = |
| | | | | 5.2, 9.6 Hz, 3H), 4.25 (s, 3H), 3.68-3.58 (m, |
| | | | | 4H), 3.26-3.21 (m, 3H), 3.14-3.00 (m, 6H), 2.95 |
| | | | | (d, J = 8.0 Hz, 3H), 2.74-2.66 (m, 1H), 2.65- |
| | | | | 2.61 (m, 3H), 2.60-2.53 (m, 2H), 2.39-2.33 (m, |
| | | | | 2H), 2.30-2.22 (m, 1H), 2.21-2.13 (m, 1H), 1.12 |
| | | | | (d, J = 5.2 Hz, 3H), 1.06-0.99 (m, 6H) |
| I-684 | JB | DR | 840.1 | 12.24 (s, 1H), 10.92 (s, 1H), 7.63-7.53 (m, |
| | | | | 2H), 7.36-7.28 (m, 2H), 7.24 (d, J = 6.8 Hz, |
| | | | | 1H), 7.10-7.01 (m, 2H), 6.64 (s, 1H), 6.16 |
| | | | | (s, 1H), 4.43-4.35 (m, 3H), 4.24 (s, 3H), |
| | | | | 3.68 (d, J = 5.6 Hz, 2H), 3.49-3.36 (m, 2H), |
| | | | | 3.24 (d, J = 4.0 Hz, 2H), 3.22-3.04 (m, 6H), |
| | | | | 3.03-2.90 (m, 2H), 2.80 (t, J = 7.6 Hz, 2H), |
| | | | | 2.75-2.66 (m, 1H), 2.66-2.62 (m, 1H), 2.62- |
| | | | | 2.53 (m, 1H), 2.36 (s, 2H), 2.35-2.31 (m, |
| | | | | 1H), 2.24 (s, 1H), 2.22-2.11 (m, 2H), 1.97- |
| | | | | 1.88 (m, 1H), 1.08-0.99 (m, 6H) |
| I-685 | KI | DR | 840.4 | 12.29-12.17 (m, 1H), 10.89 (s, 1H), 7.62- |
| | | | | 7.54 (m, 2H), 7.36-7.30 (m, 2H), 7.24 (d, J = |
| | | | | 7.2 Hz, 1H), 7.11-7.01 (m, 2H), 6.63 (s, |
| | | | | 1H), 6.13 (s, 1H), 4.42-4.35 (m, 3H), 4.29- |
| | | | | 4.22 (m, 3H), 3.76-3.59 (m, 3H), 3.48-3.39 |
| | | | | (m, 3H), 3.20-3.04 (m, 6H), 3.02-2.92 (m, |
| | | | | 2H), 2.83-2.75 (m, 2H), 2.68-2.59 (m, |
| | | | | 3H), 2.42-2.31 (m, 4H), 2.23-2.12 (m, |
| | | | | 2H), 1.97-1.86 (m, 1H), 1.08-1.00 (m, 6H) |
| I-726 | KO | AB | 926.5 | 12.32-12.19 (m, 1H), 11.12-11.01 (m, 1H), |
| | | | | 7.75-7.60 (m, 2H), 7.47-7.36 (m, 3H), 7.33- |
| | | | | 7.27 (m, 1H), 7.09-6.95 (m, 2H), 6.57- |
| | | | | 6.43 (m, 1H), 6.27-6.11 (m, 2H), 5.78-5.67 |
| | | | | (m, 1H), 4.30 (br s, 4H), 3.69-3.60 (m, 1H), |
| | | | | 3.58 (br t, J = 5.6 Hz, 2H), 3.46-3.40 (m, |
| | | | | 1H), 3.23-3.14 (m, 6H), 3.10-3.04 (m, |
| | | | | 2H), 3.02-2.95 (m, 3H), 2.89-2.73 (m, |
| | | | | 5H), 2.67 (s, 3H), 2.36-2.21 (m, 6H), 1.94- |
| | | | | 1.86 (m, 1H) |
| I-757 | GR | AB | 891.5 | 12.31 (br d, J = 4.0 Hz, 1H), 11.15-10.97 |
| | | | | (m, 1H), 7.78-7.64 (m, 1H), 7.47-7.34 (m, |
| | | | | 2H), 7.33-7.26 (m, 2H), 7.19 (d, J = 8.4 Hz, |
| | | | | 1H), 6.97 (d, J = 6.4 Hz, 1H), 6.59-6.36 (m, |
| | | | | 1H), 6.23-6.09 (m, 2H), 5.76 (s, 1H), 4.39- |
| | | | | 4.28 (m, 4H), 3.65-3.55 (m, 2H), 3.22-3.15 |
| | | | | (m, 10H), 3.02-2.93 (m, 4H), 2.77-2.70 |
| | | | | (m, 7H), 2.68-2.65 (m, 5H), 2.37-2.31 (m, |
| | | | | 2H), 2.28-2.20 (m, 2H) |
| I-751 | LK | LJ | 911.4 | 12.10 (br s, 1H), 11.08 (s, 1H), 7.72 (dd, J = |
| | | | | 1.6, 9.6 Hz, 1H), 7.48-7.39 (m, 2H), 7.32- |
| | | | | 7.25 (m, 2H), 7.18 (d, J = 8.0 Hz, 1H), 7.00- |
| | | | | 6.95 (m, 1H), 6.50 (br s, 1H), 6.24-6.16 (m, |
| | | | | 1H), 5.75 (br dd, J = 4.8, 11.6 Hz, 1H), 4.49 |
| | | | | (br d, J = 12.4 Hz, 1H), 4.40-4.32 (m, 2H), |
| | | | | 3.88 (br d, J = 11.6 Hz, 1H), 3.23-3.12 (m, |
| | | | | 10H), 3.09-3.00 (m, 4H), 2.95-2.89 (m, |
| | | | | 2H), 2.85 (br d, J = 3.2 Hz, 1H), 2.73 (br d, J = |
| | | | | 2.8 Hz, 6H), 2.66 (s, 5H), 2.59 (br s, 1H), |
| | | | | 2.24 (td, J = 4.8, 9.6 Hz, 1H), 1.97-1.82 (m, |
| | | | | 2H), 1.80-1.73 (m, 1H), 1.54-1.39 (m, 1H) |
| I-999 | ON | OM | 433.8^{b} | 12.14-12.10 (m, 1H), 10.87-10.84 (m, 1H), |
| | | | | 8.37-8.22 (m, 1H), 7.78-7.66 (m, 1H), 7.45- |
| | | | | 7.36 (m, 2H), 6.97-6.88 (m, 2H), 6.88- |
| | | | | 6.85 (m, 1H), 6.81-6.76 (m, 1H), 6.61-6.57 |
| | | | | (m, 1H), 6.52-6.47 (m, 1H), 6.22-6.16 (m, |
| | | | | 1H), 6.13-6.08 (m, 1H), 5.29-5.25 (m, |
| | | | | 1H), 4.39-4.32 (m, 4H), 4.32-4.25 (m, |
| | | | | 2H), 3.78 (s, 3H), 3.66-3.62 (m, 1H), 3.60- |
| | | | | 3.54 (m, 2H), 3.26 (br s, 4H), 3.17-3.03 (m, |
| | | | | 6H), 2.98-2.93 (m, 2H), 2.87-2.74 (m, |
| | | | | 2H), 2.67 (br s, 2H), 2.61-2.57 (m, 4H), |
| | | | | 2.34-2.29 (m, 2H), 2.16-2.10 (m, 1H), 1.98- |
| | | | | 1.91 (m, 1H), 1.97-1.91 (m, 1H) |
| I-1002 | MJ | AB | 910.6 | 12.4-12. (m, 1H), 11.38-10.75 (m, 1H), |
| | | | | 7.71 (dd, J = 1.8, 11.4 Hz, 1H), 7.52-7.44 |
| | | | | (m, 2H), 7.44-7.35 (m, 2H), 7.29 (dd, J = |
| | | | | 7.2, 8.4 Hz, 1H), 7.15-7.05 (m, 1H), 6.97 |
| | | | | (d, J = 8.0 Hz, 1H), 6.65 (dd, J = 2.8, 5.2 Hz, |
| | | | | 1H), 6.19 (td, J = 2.0, 14.1 Hz, 2H), 5.73 (dd, |
| | | | | J = 5.2, 12 Hz, 1H), 4.37 (br t, J = 6.8 Hz, |
| | | | | 3H), 4.32 (br d, J = 12.8 Hz, 1H), 3.67-3.41 |
| | | | | (m, 4H), 3.24-3.13 (m, 6H), 3.02-2.93 (m, |
| | | | | 3H), 2.90-2.69 (m, 6H), 2.67 (s, 4H), 2.45- |
| | | | | 2.39 (m, 1H), 2.37-2.18 (m, 5H), 1.94-1.84 |
| | | | | (m, 1H) |
| I-1003 | MK | AB | 910.4 | 12.4-12.0 (m, 1H), 11.05 (br s, 1H), 7.71 (dd, J = |
| | | | | 2.0, 11.2 Hz, 1H), 7.52-7.44 (m, 2H), 7.43- |
| | | | | 7.36 (m, 2H), 7.32-7.26 (m, 1H), 7.14-7.05 (m, |
| | | | | 1H), 6.97 (d, J = 6.8 Hz, 1H), 6.68-6.62 (m, |
| | | | | 1H), 6.22-6.14 (m, 2H), 5.73 (dd, J = 5.2, 11.6 |
| | | | | Hz, 1H), 4.40-4.35 (m, 3H), 4.32 (br d, J = 12.4 |
| | | | | Hz, 1H), 3.68-3.41 (m, 4H), 3.24-3.13 (m, |
| | | | | 6H), 2.97 (q, J = 7.2 Hz, 3H), 2.91-2.69 (m, |
| | | | | 6H), 2.67 (s, 4H), 2.45-2.39 (m, 1H), 2.39- |
| | | | | 2.17 (m, 5H), 1.93-1.84 (m, 1H) |
| I-1004 | GR | NG | 920.4 | 12.25-12.14 (m, 1H), 11.08 (s, 1H), 7.76-7.68 |
| | | | | (m, 1H), 7.45-7.35 (m, 1H), 7.34-7.28 (m, |
| | | | | 1H), 7.23-7.15 (m, 2H), 6.99-6.90 (m, 2H), |
| | | | | 6.89-6.79 (m, 1H), 6.51 (s, 1H), 6.22-6.10 (m, |
| | | | | 2H), 5.31-5.14 (m, 1H), 4.39-4.28 (m, 4H), |
| | | | | 3.66-3.55 (m, 2H), 3.16 (s, 7H), 3.04 (s, 2H), |
| | | | | 2.97 (q, J = 7.2 Hz, 3H), 2.92-2.83 (m, 3H), |
| | | | | 2.69 (s, 4H), 2.65-2.58 (m, 4H), 2.35-2.24 (m, |
| | | | | 2H), 1.96 (dd, J = 4.8, 10.4 Hz, 1H), 1.41 (d, J = |
| | | | | 3.2 Hz, 6H) |
| I-1005 | GR | NJ | 918.5 | 12.69-11.53 (m, 1H), 11.50-10.35 (m, 1H), |
| | | | | 7.75-7.67 (m, 1H), 7.47-7.34 (m, 1H), 7.33- |
| | | | | 7.26 (m, 1H), 7.19-7.14 (m, 2H), 6.96-6.86 (m, |
| | | | | 3H), 6.50 (d, J = 3.2 Hz, 1H), 6.22-6.10 (m, |
| | | | | 2H), 5.40-5.22 (m, 1H), 4.38-4.28 (m, 4H), |
| | | | | 3.66-3.53 (m, 5H), 3.14 (br s, 6H), 3.01-2.84 |
| | | | | (m, 5H), 2.70-2.60 (m, 8H), 2.38-2.21 (m, |
| | | | | 3H), 1.98 (br d, J = 4.0 Hz, 3H), 1.53-1.46 (m, |
| | | | | 2H) |
| I-1006 | PJ | NG | 919.5 | 12.32-12.15 (m, 1H), 11.20-10.93 (m, 1H), |
| | | | | 7.75-7.66 (m, 1H), 7.59-7.52 (m, 1H), 7.44- |
| | | | | 7.31 (m, 2H), 7.22-7.16 (m, 1H), 7.03-6.90 (m, |
| | | | | 2H), 6.88-6.79 (m, 1H), 6.52-6.46 (m, 1H), |
| | | | | 6.22-6.12 (m, 2H), 5.35-5.12 (m, 1H), 4.39- |
| | | | | 4.28 (m, 4H), 3.65-3.54 (m, 2H), 3.16-3.11 (m, |
| | | | | 4H), 3.08-2.93 (m, 6H), 2.91-2.82 (m, 4H), |
| | | | | 2.68-2.59 (m, 4H), 2.32 (br d, J = 2.0 Hz, 2H), |
| | | | | 2.26-2.20 (m, 2H), 1.98-1.92 (m, 1H), 1.87- |
| | | | | 1.79 (m, 4H), 1.42-1.38 (m, 6H) |
| I-993 | PV | PW | 1021.6 | 11.95 (s, 1H), 8.15-8.05 (m, 1H), 7.75-7.65 |
| | | | | (m, 2H), 7.25-7.19 (m, 1H), 7.19-7.14 (m, |
| | | | | 1H), 6.90-6.73 (m, 3H), 6.66 (s, 1H), 6.62- |
| | | | | 6.55 (m, 1H), 6.51-6.42 (m, 2H), 6.20-6.07 |
| | | | | (m, 2H), 5.70 (s, 1H), 4.68-4.57 (m, 2H), 4.51- |
| | | | | 4.40 (m, 1H), 4.36 (s, 1H), 4.31 (s, 1H), 4.19- |
| | | | | 4.12 (m, 1H), 3.89-3.81 (m, 2H), 3.74-3.68 |
| | | | | (m, 3H), 3.68-3.56 (m, 3H), 3.55-3.48 (m, |
| | | | | 1H), 3.46-3.37 (m, 2H), 3.14-3.04 (m, 5H), |
| | | | | 2.85-2.69 (m, 4H), 2.54 (s, 8H), 2.38-2.28 (m, |
| | | | | 4H), 2.27 (s, 1H), 2.08-1.99 (m, 1H), 1.99- |
| | | | | 1.87 (m, 1H), 1.77-1.69 (m, 3H), 1.51 (s, 4H), |
| | | | | 1.29-1.20 (m, 4H), 1.20-1.10 (m, 2H) |
| I-994 | PV | PY | 1063.6 | 11.94 (d, J = 8.4 Hz, 1H), 8.11 (d, J = 10.4 Hz, |
| | | | | 1H), 7.74-7.63 (m, 2H), 7.25-7.13 (m, 2H), |
| | | | | 6.95-6.71 (m, 3H), 6.66 (s, 1H), 6.59 (d, J = 8.6 |
| | | | | Hz, 1H), 6.53-6.41 (m, 2H), 6.19-6.01 (m, |
| | | | | 2H), 5.69 (s, 1H), 4.70-4.56 (m, 2H), 4.45 (d, J = |
| | | | | 11.5 Hz, 1H), 4.36 (s, 1H), 4.31 (s, 1H), 4.15 |
| | | | | (s, 1H), 3.84 (d, J = 12.9 Hz, 2H), 3.71 (d, J = 4.0 |
| | | | | Hz, 3H), 3.69-3.56 (m, 2H), 3.56-3.46 (m, |
| | | | | 2H), 3.46-3.33 (m, 2H), 3.31-3.16 (m, 5H), |
| | | | | 3.16-2.97 (m, 3H), 2.75 (td, J = 22.9, 20.4, 10.7 |
| | | | | Hz, 3H), 2.38-2.31 (m, 4H), 2.33-2.17 (m, |
| | | | | 2H), 2.14-1.97 (m, 1H), 1.97-1.84 (m, 1H), |
| | | | | 1.82-1.61 (m, 4H), 1.58-1.40 (m, 5H), 1.38- |
| | | | | 1.26 (m, 6H), 1.30-1.17 (m, 6H), 1.19-1.04 |
| | | | | (m, 3H) |
| I-995 | RY | RX | 1078.7 | 11.95 (d, J = 8.3 Hz, 1H), 8.12 (t, J = 10.4 Hz, |
| | | | | 1H), 7.73-7.65 (m, 2H), 7.19 (t, J = 7.5 Hz, |
| | | | | 2H), 6.91-6.72 (m, 3H), 6.66 (s, 1H), 6.63- |
| | | | | 6.56 (m, 1H), 6.47 (s, 2H), 6.24 (s, 1H), 6.09 (s, |
| | | | | 1H), 5.73 (s, 1H), 4.68-4.57 (m, 2H), 4.45 (s, |
| | | | | 1H), 4.36 (s, 1H), 4.30 (s, 1H), 4.14 (s, 1H), 3.90 |
| | | | | (d, J = 12.8 Hz, 3H), 3.71 (d, J = 4.0 Hz, 4H), |
| | | | | 3.65 (s, 1H), 3.60 (s, 1H), 3.52 (s, 1H), 3.25 (s, |
| | | | | 3H), 3.23 (s, 1H), 3.13-3.06 (m, 2H), 2.80 (t, J = |
| | | | | 12.2 Hz, 5H), 2.58 (d, J = 5.6 Hz, 5H), 2.43 (s, |
| | | | | 2H), 2.34 (s, 1H), 2.27 (s, 1H), 2.19 (s, 1H), 2.03 |
| | | | | (s, 2H), 1.93 (t, J = 11.0 Hz, 1H), 1.75 (s, 6H), |
| | | | | 1.53 (d, J = 28.5 Hz, 6H), 1.24 (s, 2H), 1.18 (d, J = |
| | | | | 11.2 Hz, 6H), 0.86 (t, J = 6.6 Hz, 1H) |
| I-996^{d} | PV | RJ | 1290.9 | 8.01 (d, J = 7.5 Hz, 1H), 7.76 (d, J = 2.6 Hz, 1H), |
| | | | | 7.70 (d, J = 9.8 Hz, 1H), 7.40-7.37 (m, 2H), |
| | | | | 7.36-7.26 (m, 3H), 7.24-7.18 (m, 3H), 6.92 |
| | | | | (dd, J = 6.3, 4.3 Hz, 1H), 6.78-6.67 (m, 2H), |
| | | | | 6.70-6.61 (m, 2H), 6.59 (dq, J = 9.0, 4.9, 4.3 |
| | | | | Hz, 1H), 6.18-6.10 (m, 1H), 5.56 (d, J = 26.0 |
| | | | | Hz, 1H), 5.62-5.46 (m, 1H), 4.46 (d, J = 2.6 Hz, |
| | | | | 1H), 4.37 (d, J = 2.5 Hz, 1H), 4.00 (s, 3H), 3.77 |
| | | | | (d, J = 5.9 Hz, 1H), 3.75 (s, 3H), 3.72-3.52 (m, |
| | | | | 7H), 3.31 (d, J = 4.5 Hz, 2H), 3.23-3.07 (m, |
| | | | | 6H), 2.91 (d, J = 6.5 Hz, 3H), 2.82 (t, J = 4.9 Hz, |
| | | | | 4H), 2.44-2.30 (m, 5H), 2.08-1.99 (m, 2H), |
| | | | | 1.88 (dd, J = 16.6, 2.6 Hz, 3H), 1.67 (qd, J = |
| | | | | 12.5, 3.7 Hz, 2H), 1.31 (s, 3H), 0.96-0.85 (m, |
| | | | | 2H) |
| I-997 | PV | QZ | 1325.0 | 12.48 (s, 1H), 11.94 (d, J = 8.6 Hz, 1H), 8.29 (d, |
| | | | | J = 7.0 Hz, 1H), 8.15-8.08 (m, 1H), 7.90 (d, J = |
| | | | | 5.1 Hz, 1H), 7.69 (d, J = 11.8 Hz, 1H), 7.37- |
| | | | | 7.14 (m, 8H), 7.06 (d, J = 7.9 Hz, 2H), 6.92- |
| | | | | 6.80 (m, 3H), 6.66-6.68 (m, 2H), 6.58 (dd, J = |
| | | | | 8.5, 2.2 Hz, 1H), 6.49 (s, 1H), 6.14-6.06 (m, |
| | | | | 1H), 5.20-5.23 (m, 1H), 4.62-4.64 (m, 2H), 4.37 |
| | | | | (s, 1H), 4.33-4.28 (m, 1H), 3.73-3.62 (m, 4H), |
| | | | | 3.62-3.50 (m, 6H), 3.51 (d, J = 6.9 Hz, 2H), |
| | | | | 3.33 (s, 1H), 3.22 (t, J = 4.9 Hz, 4H), 3.08-3.10 |
| | | | | (m, 2H), 2.74 (t, J = 6.9 Hz, 2H), 2.63-2.51 (m, |
| | | | | 8H), 2.35 (s, 1H), 2.27 (s, 1H), 1.86 (d, J = 2.4 |
| | | | | Hz, 2H), 1.75 (d, J = 2.4 Hz, 2H), 1.25 (d, J = 3.9 |
| | | | | Hz, 1H) |
| I-998 | PV | RB | 1369.6 | 12.48 (s, 1H), 11.95 (d, J = 8.9 Hz, 1H), 8.29 (s, |
| | | | | 1H), 8.11 (d, J = 10.7 Hz, 1H), 7.89 (s, 1H), 7.69 |
| | | | | (d, J = 12.0 Hz, 1H), 7.38-7.18 (m, 9H), 7.17 |
| | | | | (d, J = 8.2 Hz, 1H), 7.06 (d, J = 7.8 Hz, 2H), 6.85 |
| | | | | (q, J = 8.1, 6.9 Hz, 3H), 6.65 (s, 2H), 6.58 (d, J = |
| | | | | 8.9 Hz, 1H), 6.48 (s, 1H), 6.10 (d, J = 11.5 Hz, |
| | | | | 1H), 5.23-5.18 (m, 1H), 4.64 (t, J = 7.4 Hz, |
| | | | | 2H), 4.33 (d, J = 23.3 Hz, 2H), 3.74-3.62 (m, |
| | | | | 5H), 3.58-3.48 (m, 23H), 3.09 (q, J = 7.4 Hz, |
| | | | | 12H), 2.73 (t, J = 7.0 Hz, 2H), 2.59 (d, J = 5.4 |
| | | | | Hz, 5H), 2.30 (d, J = 28.6 Hz, 3H), 1.85 (d, J = |
| | | | | 2.4 Hz, 1H), 1.74 (d, J = 2.4 Hz, 1H) |
| I-1000 | PV | RP | 1148.6 | 12.01-11.93 (m, 1H), 8.11 (d, J = 10.5 Hz, 1H), |
| | | | | 7.76-7.66 (m, 2H), 7.16 (d, J = 8.3 Hz, 1H), 6.91- |
| | | | | 6.80 (m, 1H), 6.79-6.68 (m, 3H), 6.65 (s, 1H), |
| | | | | 6.57 (d, J = 8.6 Hz, 2H), 6.51-6.40 (m, 2H), 6.14- |
| | | | | 6.04 (m, 1H), 5.98 (d, J = 6.1 Hz, 1H), 5.48 (s, |
| | | | | 1H), 4.91 (s, 2H), 4.67-4.56 (m, 2H), 4.51-4.39 |
| | | | | (m, 1H), 4.37-4.27 (m, 2H), 4.19-4.11 (m, 1H), |
| | | | | 3.70 (s, 3H), 3.67-3.57 (m, 2H), 3.55-3.47 (m, |
| | | | | 3H), 3.46-3.39 (m, 4H), 3.39-3.36 (m, 5 H), 3.26- |
| | | | | 3.16 (m, 7H), 3.13-2.97 (m, 6H), 2.84-2.73 (m, |
| | | | | 1H), 2.37-2.22 (m, 8H), 2.08-2.01 (m, 1H), 1.95- |
| | | | | 1.86 (m, 1H), 1.80-1.71 (m 1H), 1.66-1.60 (m, |
| | | | | 2H), 1.52-1.42 (m, 2H), 1.3-1.28 (m, 8H) |
| I-1001 | PV | RO | 1148.7 | 11.97-11.89 (m, 1H), 8.20-8.03 (m, 1H), 7.80- |
| | | | | 7.64 (m, 2H), 7.22-7.10 (m, 1H), 6.98-6.41 (m, |
| | | | | 9H), 6.21-5.93 (m, 2H), 5.49 (s, 1H), 4.97-4.89 |
| | | | | (m, 1H), 4.70-4.58 (m, 2H), 4.46-4.29 (m, 3H), |
| | | | | 4.25-4.12 (m, 2H), 3.76-3.56 (m, 7H), 3.55-3.40 |
| | | | | (m, 10H), 3.24-3.00 (m, 14H), 2.36-2.19 (m, |
| | | | | 6H), 2.07-1.89 (m, 3H), 1.80-1.59 (m, 2H), 1.52- |
| | | | | 1.42 (m, 2H), 1.32-1.23 (m, 10H) |
| I-1422 | ST | NG | 886.3 | 12.24 (d, J = 1.2 Hz, 1H), 11.06 (s, 1H), 8.11 (d, |
| | | | | J = 11.6 Hz, 1H), 7.77-7.60 (m, 1H), 7.55-7.37 |
| | | | | (m, 3H), 7.19 (t, J = 7.6 Hz, 1H), 7.14-7.02 (m, |
| | | | | 1H), 6.94 (d, J = 8.0 Hz, 1H), 6.88 (s, 1H), 6.86- |
| | | | | 6.77 (m, 1H), 6.18 (s, 1H), 5.30-5.16 (m, 1H), |
| | | | | 4.71-4.57 (m, 2H), 4.42-4.27 (m, 2H), 3.71- |
| | | | | 3.55 (m, 2H), 3.18 (d, J = 4.4 Hz, 2H), 3.15- |
| | | | | 3.03 (m, 7H), 2.91-2.83 (m, 4H), 2.65-2.58 (m, |
| | | | | 4H), 2.34 (d, J = 10.0 Hz, 2H), 2.31-2.25 (m, |
| | | | | 1H), 2.24-2.14 (m, 2H), 2.00-1.91 (m, 1H), |
| | | | | 1.80 (s, 4H), 1.40 (d, J = 2.8 Hz, 6H) |
| I-1423 | SU | NG | 882.5 | 12.13 (d, J = 7.6 Hz, 1H), 11.07 (s, 1H), 8.14- |
| | | | | 8.06 (m, 1H), 7.72-7.64 (m, 1H), 7.27-7.10 (m, |
| | | | | 4H), 6.94 (d, J = 7.6 Hz, 1H), 6.88 (d, J = 6.0 Hz, |
| | | | | 1H), 6.83-6.75 (m, 1H), 6.40-6.34 (m, 1H), |
| | | | | 6.19-6.10 (m, 1H), 5.32-5.12 (m, 1H), 4.66- |
| | | | | 4.56 (m, 2H), 4.40-4.28 (m, 2H), 3.68-3.55 (m, |
| | | | | 3H), 3.14-3.03 (m, 8H), 2.87 (t, J = 7.6 Hz, 3H), |
| | | | | 2.64 (s, 1H), 2.63-2.55 (m, 4H), 2.55-2.52 (m, |
| | | | | 1H), 2.33 (s, 1H), 2.26 (s, 1H), 2.22-2.15 (m, |
| | | | | 2H), 2.14 (s, 3H), 1.99-1.91 (m, 1H), 1.87- |
| | | | | 1.68 (m, 4H), 1.40 (d, J = 3.2 Hz, 6H) |
| I-1424^{b} | SV | NG | 441.4 | 12.15-12.08 (m, 1H), 11.06 (s, 1H), 7.71 (dd, J = |
| | | | | 2.0, 10.8 Hz, 1H), 7.45-7.35 (m, 1H), 7.24- |
| | | | | 7.13 (m, 4H), 6.94 (d, J = 8.0 Hz, 1H), 6.89- |
| | | | | 6.78 (m, 2H), 6.37 (d, J = 2.0 Hz, 1H), 6.22- |
| | | | | 6.10 (m, 2H), 5.28-5.18 (m, 1H), 4.40-4.27 (m, |
| | | | | 4H), 3.63 (t, J = 5.6 Hz, 1H), 3.57 (br t, J = 5.2 |
| | | | | Hz, 1H), 3.11 (d, J = 10.8 Hz, 6H), 3.01-2.94 |
| | | | | (m, 3H), 2.87 (t, J = 7.6 Hz, 3H), 2.68-2.57 (m, |
| | | | | 5H), 2.36-2.24 (m, 3H), 2.20-2.13 (m, 5H), |
| | | | | 2.00-1.92 (m, 1H), 1.87-1.80 (m, 2H), 1.79- |
| | | | | 1.68 (m, 2H), 1.40 (d, J = 3.2 Hz, 6H) |
| I-1425^{b} | SW | NG | 443.2 | 12.23 (d, J = 7.2 Hz, 1H), 11.06 (s, 1H), 7.74- |
| | | | | 7.69 (m, 1H), 7.52-7.36 (m, 4H), 7.19 (t, J = 8.0 |
| | | | | Hz, 1H), 7.12-7.03 (m, 1H), 6.94 (d, J = 8.0 Hz, |
| | | | | 1H), 6.88 (s, 1H), 6.86-6.81 (m, 1H), 6.19 (d, J = |
| | | | | 13.6 Hz, 2H), 5.31-5.15 (m, 1H), 4.41-4.27 |
| | | | | (m, 4H), 3.65 (t, J = 5.6 Hz, 1H), 3.58 (t, J = 5.6 |
| | | | | Hz, 1H), 3.15 (d, J = 10.0 Hz, 6H), 3.03-2.93 |
| | | | | (m, 3H), 2.93-2.82 (m, 4H), 2.68-2.58 (m, |
| | | | | 4H), 2.38-2.18 (m, 5H), 2.01-1.91 (m, 1H), |
| | | | | 1.81 (s. 4H), 1.40 (d, J = 3.2 Hz, 6H) |
| I-1442 | TV | NG | 867.4 | 12.20-12.18 (m, 1H), 11.07 (s, 1H), 7.73-7.70 |
| | | | | (m, 1H), 7.60 (d, J = 8.0 Hz, 2H), 7.43-7.37 (m, |
| | | | | 3H), 7.21-7.17 (m, 1H), 7.06-6.99 (m, 1H), |
| | | | | 6.94 (d, J = 7.6 Hz, 1H), 6.89-6.80 (m, 2H), |
| | | | | 6.21-6.15 (m, 2H), 5.23 (s, 1H), 4.39-4.30 (m, |
| | | | | 4H), 3.65 (t, J = 5.6 Hz, 1H), 3.59-3.56 (m, 1H), |
| | | | | 3.22-3.04 (m, 7H), 3.00-2.95 (m, 3H), 2.90- |
| | | | | 2.86 (m, 3H), 2.67-2.56 (m, 5H), 2.33-2.27 (m, |
| | | | | 2H), 2.18 (t, J = 10.4 Hz, 2H), 1.96-1.94 (m, |
| | | | | 1H), 1.85-1.83 (m, 2H), 1.78-1.70 (m, 2H), |
| | | | | 1.40 (d, J = 3.2 Hz, 6H) |

DCE could be used as the solvent;
^{a}The reductive amination was run under a variety of standard conditions for 2-16 hrs at 0-40° C. Other reagents employed included: TEA or AcONa as base, NaBH(OAc)₃ as the reducing agent, MeOH, DMSO or DCE as solvent. 4 Å molecular sieves could also be added to the mixture. Work up also by direct concentration, dilution with water and extraction with EtOAc. Organic layer washed with brine, dried over Na₂SO₄, filtration and concentration. Purification by Prep-TLC, Prep- HPLC, and reverse phase flash chromatography under a variety of solvent conditions. Standard deprotection conditions such as HCl/dioxane or TFA/DCM could also be employed to deprotect final products when applicable.
^{b}LC-MS (ESI⁺) m/z reported as (M/2 + H)⁺.
^{d}Ketone instead of aldehyde used for the reductive amination.

TABLE 4
Compounds synthesized via Method 1, the reductive amination of the amines and aldehydes

| | LCMS | |
|---|---|---|
| | (ESI+) m/z | |
| I-# | (M + H)+ | ¹H NMR (400 MHz, DMSO-d₆) δ |
| I-63 | 847.5 | 11.24-10.98 (m, 1H), 8.24 (s, 1H), 8.11 (s, 1H), 7.70-7.60 (m, 2H), 7.59- |
| | | 7.53 (m, 1H), 7.40-7.35 (m, 1H), 6.96-6.91 (m, 2H), 6.86-6.83 (m, 1H), |
| | | 6.34-6.24 (m, 1H), 5.39-5.31 (m, 1H), 4.63 (t, J = 6.8 Hz, 2H), 4.40-4.34 |
| | | (m, 2H), 4.02-3.96 (m, 2H), 3.61-3.57 (m, 2H), 3.54 (s, 3H), 3.26 (br s, 3H), |
| | | 3.14-3.10 (m, 2H), 3.05-3.00 (m, 3H), 2.89-2.84 (m, 3H), 2.70-2.62 (m, |
| | | 2H), 2.59-2.55 (m, 2H), 2.36-2.30 (m, 2H), 2.28-2.23 (m, 1H), 2.01-1.96 |
| | | (m, 1H), 1.60-1.55 (m, 2H), 1.42 (br d, J = 6.8 Hz, 2H), 1.38-1.34 (m, 2H), |
| | | 1.28-1.21 (m, 14H) |
| I-97 | 892.4 | 11.56-11.44 (m, 1H), 11.12 (s, 1H), 8.25 (s, 1H), 8.12 (s, 1H), 7.72-7.66 (m, |
| | | 1H), 7.65-7.55 (m, 2H), 7.46 (d, J = 1.6 Hz, 1H), 7.38-7.21 (m, 4H), 7.17 (d, |
| | | J = 8.4 Hz, 1H), 7.13-7.08 (m, 1H), 7.02 (d, J = 1.6 Hz, 1H), 6.34-6.20 (m, |
| | | 1H), 5.39 (dd, J = 5.2, 12.8 Hz, 1H), 4.64 (t, J = 6.8 Hz, 2H), 4.44-4.28 (m, |
| | | 2H), 3.73 (s, 2H), 3.63-3.57 (m, 2H), 3.17-3.08 (m, 6H), 2.97-2.87 (m, 2H), |
| | | 2.74 (dd, J = 4.0, 12.4 Hz, 2H), 2.70-2.63 (m, 2H), 2.63-2.59 (m, 1H), 2.46- |
| | | 2.42 (m, 6H), 2.38-2.32 (m, 6H), 2.31-2.12 (m, 4H), 2.12-1.97 (m, 2H), |
| | | 1.81-1.63 (m, 2H) |
| I-106 | 884.8 | 11.62-11.36 (m, 1H), 11.23-10.87 (m, 1H), 8.29 (s, 1H), 8.18-8.09 (m, 1H), |
| | | 7.77-7.62 (m, 1H), 7.35-7.26 (m, 1H), 7.20-7.15 (m, 1H), 7.03 (s, 1H), 7.01- |
| | | 6.92 (m, 2H), 6.86 (d, J = 8.4 Hz, 1H), 6.31-6.17 (m, 1H), 5.34-5.29 (m, |
| | | 1H), 4.75-4.56 (m, 2H), 4.46-4.26 (m, 2H), 4.03-3.95 (m, 2H), 3.77-3.72 |
| | | (m, 1H), 3.63-3.53 (m, 10H), 3.50 (s, 3H), 3.28 (s, 6H), 3.14-3.07 (m, 4H), |
| | | 2.97-2.75 (m, 6H), 2.74-2.59 (m, 6H), 2.34-2.24 (m, 2H), 2.02-1.96 (m, |
| | | 2H) |
| I-107 | 971.4 | 11.60-11.42 (m, 1H), 11.38-10.67 (m, 1H), 8.62-8.17 (m, 1H), 8.12 (s, 1H), |
| | | 7.69 (s, 1H), 7.39-7.24 (m, 1H), 7.18 (s, 1H), 7.06 (s, 1H), 7.03-6.93 (m, |
| | | 2H), 6.89 (d, J = 8.0 Hz, 1H), 6.26 (d, J = 3.6 Hz, 1H), 5.33 (dd, J = 5.2, 12.0 |
| | | Hz, 1H), 4.63 (t, J = 6.4 Hz, 2H), 4.49-4.24 (m, 2H), 4.19-3.85 (m, 2H), 3.83- |
| | | 3.73 (m, 1H), 3.69-3.54 (m, 8H), 3.49 (s, 3H), 3.46 (d, J = 3.2 Hz, 8H), 3.31 |
| | | (s, 6H), 3.16-3.06 (m, 8H), 2.82 (t, J = 6.8 Hz, 4H), 2.76-2.55 (m, 6H), 2.32 |
| | | (d. J = 2.8 Hz, 2H), 2.03-1.95 (m, 2H) |
| I-108 | 971.3 | 11.69-11.38 (m, 1H), 11.37-10.66 (m, 1H), 8.28 (s, 1H), 8.23-7.99 (m, 1H), |
| | | 7.83-7.56 (m, 1H), 7.32-7.18 (m, 3H), 7.01-6.92 (m, 3H), 6.41-6.06 (m, |
| | | 1H), 5.35 (dd, J = 4.8, 12.4 Hz, 1H), 4.74-4.53 (m, 2H), 4.47-4.22 (m, 2H), |
| | | 4.08-3.94 (m, 2H), 3.88-3.78 (m, 1H), 3.65 (s, 6H), 3.56 (s, 3H), 3.48 (s, |
| | | 4H), 3.44 (s, 6H), 3.34-3.33 (m, 6H), 3.12 (dd, J = 6.4, 13.2 Hz, 8H), 3.01- |
| | | 2.79 (m, 4H), 2.78-2.55 (m, 6H), 2.35-2.25 (m, 2H), 2.05-1.83 (m, 2H) |
| I-7 | 997.2 | 8.98 (s, 1H), 8.60-8.53 (m, 1H), 8.15-8.10 (m, 1H), 7.88 (s, 1H), 7.74-7.53 |
| | | (m, 5H), 7.45-7.36 (m, 5H), 6.71 (d, J = 8.8 Hz, 2H), 6.42-6.32 (m, 1H), 4.68- |
| | | 4.61 (m, 2H), 4.59-4.55 (m, 1H), 4.48-4.39 (m, 4H), 4.36 (s, 1H), 4.27- |
| | | 4.17 (m, 1H), 3.71-3.57 (m, 4H), 3.41-3.37 (m, 7H), 3.32-3.30 (m, 6H), |
| | | 3.20-2.97 (m, 9H), 2.44 (s, 3H), 1.66-1.51 (m, 4H), 1.01-0.89 (m, 9H) |
| I-8 | 1025.4 | 8.97 (s, 1H), 8.60-8.51 (m, 1H), 8.14-8.11 (m, 1H), 7.86 (d, J = 9.2 Hz, 1H), |
| | | 7.72-7.52 (m, 5H), 7.44-7.36 (m, 5H), 6.75-6.68 (m, 2H), 6.41-6.31 (m, |
| | | 1H), 4.64 (t, J = 7.2 Hz, 2H), 4.55 (d, J = 9.6 Hz, 1H), 4.35 (s, 5H), 4.21 (dd, J = |
| | | 5.6, 15.6 Hz, 1H), 3.67-3.56 (m, 4H), 3.28-3.25 (m, 6H), 3.13-3.02 (m, |
| | | 6H), 2.43 (s, 3H), 2.30-2.23 (m, 2H), 2.17-2.11 (m, 1H), 2.06-1.99 (m, 1H), |
| | | 1.90 (ddd J = 4.4, 8.4, 12.8 Hz, 1H), 1.61-1.47 (m, 4H), 1.44-1.22 (m, 5H), |
| | | 0.97-0.90 (m, 9H) |
| I-12 | 1079.4 | 8.97 (d, J = 3.2 Hz, 1H), 8.56 (t, J = 6.0 Hz, 1H), 8.17-8.06 (m, 1H), 7.91 (dd, |
| | | J = 2.4, 9.2 Hz, 1H), 7.85-7.58 (m, 5H), 7.49-7.35 (m, 6H), 6.48-6.31 (m, |
| | | 1H), 4.71-4.50 (m, 4H), 4.48-4.31 (m, 5H), 4.21 (dd, J = 5.6, 16.0 Hz, 1H), |
| | | 3.97 (d, J = 12.4 Hz, 1H), 3.67 (s, 1H), 3.65-3.58 (m, 2H), 3.26 (s, 6H), 3.15 |
| | | (d, J = 6.8 Hz, 2H), 3.02 (s, 3H), 2.90-2.80 (m, 1H), 2.69-2.58 (m, 1H), 2.43 |
| | | (s, 3H), 2.38-2.29 (m, 4H), 2.29-2.14 (m, 3H), 2.08-1.99 (m, 1H), 1.96- |
| | | 1.79 (m, 3H), 1.79-1.68 (m, 2H), 1.64-1.41 (m, 2H), 1.07-0.78 (m, 9H) |
| I-33 | 832.5 | 10.77 (s, 1H), 8.14-8.10 (m, 1H), 7.74-7.49 (m, 2H), 7.45-7.33 (m, 2H), |
| | | 7.05 (d, J = 8.8 Hz, 2H), 6.88 (d, J = 8.8 Hz, 2H), 6.29 (d, J = 16.8 Hz, 1H), |
| | | 4.63 (t, J = 6.8 Hz, 2H), 4.37 (d, J = 12.8 Hz, 3H), 3.85 (d, J = 13.6 Hz, 1H), |
| | | 3.72 (dd, J = 4.8, 10.8 Hz, 1H), 3.62-3.55 (m, 2H), 3.29-3.16 (m, 6H), 3.15- |
| | | 3.06 (m, 6H), 3.04-2.92 (m, 3H), 2.89 (t, J = 6.8 Hz, 2H), 2.65 (d, J = 5.6 |
| | | Hz, 3H), 2.63-2.57 (m, 2H), 2.48-2.42 (m, 1H), 2.39-2.29 (m, 3H), 2.25 (s, |
| | | 1H), 2.19-2.07 (m, 1H), 2.05-1.95 (m, 1H), 1.91-1.67 (m, 4H), 1.64-1.42 |
| | | (m, 2H), 1.38-1.12 (m, 2H) |
| I-117 | 760.3 | 10.78 (s, 1H), 8.11 (s, 1H), 7.80-7.66 (m, 2H), 7.59-7.48 (m, 1H), 7.40 (d, J = |
| | | 7.6 Hz, 1H), 7.12 (d, J = 8.8 Hz, 2H), 6.92 (br d, J = 8.0 Hz, 2H), 6.33 (br d, |
| | | J = 11.8 Hz, 1H), 4.67-4.55 (m, 3H), 4.36 (br d, J = 8.0 Hz, 2H), 3.95-3.83 |
| | | (m, 1H), 3.81-3.71 (m, 2H), 3.58 (td, J = 5.6, 17.4 Hz, 2H), 3.38 (br d, J = 3.6 |
| | | Hz, 2H), 3.27-3.21 (m, 3H), 3.17-3.07 (m, 3H), 3.03 (br s, 2H), 2.74 (br s, |
| | | 4H), 2.70-2.60 (m, 1H), 2.32 (br d, J = 2.0 Hz, 1H), 2.27-2.20 (m, 1H), 2.18- |
| | | 2.07 (m, 1H), 2.05-1.93 (m, 2H), 1.93-1.85 (m, 1H), 1.68-1.46 (m, 2H) |
| I-121 | 808.4 | 10.79 (s, 1H), 8.12 (d, J = 1.6 Hz, 1H), 7.70 (d, J = 2.4 Hz, 1H), 7.41-7.33 (m, |
| | | 1H), 7.31-7.22 (m, 1H), 7.05-6.96 (m, 1H), 6.91 (d, J = 16.0 Hz, 1H), 6.47 |
| | | (s, 1H), 6.46-6.42 (m, 1H), 6.27 (d, J = 14.0 Hz, 1H), 6.04 (d, J = 8.4 Hz, 1H), |
| | | 4.64 (t, J = 6.8 Hz, 2H), 4.61-4.53 (m, 1H), 4.38 (s, 1H), 4.36-4.28 (m, 2H), |
| | | 4.16-4.09 (m, 1H), 3.92-3.83 (m, 2H), 3.62 (t, J = 5.6 Hz, 1H), 3.57 (t, J = |
| | | 5.6 Hz, 1H), 3.17-3.08 (m, 4H), 3.07-2.96 (m, 3H), 2.90 (d, J = 8.4 Hz, 3H), |
| | | 2.81-2.65 (m, 2H), 2.63-2.56 (m, 2H), 2.37-2.28 (m, 2H), 2.28-2.20 (m, |
| | | 1H), 2.13-2.04 (m, 1H), 1.94-1.65 (m, 8H), 1.63-1.52 (m, 1H), 1.49-1.38 |
| | | (m, 1H) |
| I-123 | 958.7 | 11.27-10.85 (m, 1H), 8.31-7.64 (m, 2H), 7.48-7.20 (m, 2H), 7.11-6.95 (m, |
| | | 2H), 6.95-6.80 (m, 2H), 6.38-6.18 (m, 1H), 5.33 (dd, J = 5.2, 12.4 Hz, 1H), |
| | | 4.63 (t, J = 6.8 Hz, 2H), 4.35 (d, J = 14.8 Hz, 2H), 3.75-3.53 (m, 8H), 3.32 (s, |
| | | 6H), 3.26 (s, 6H), 3.15-3.09 (m, 2H), 3.06-2.98 (m, 3H), 2.92-2.83 (m, 1H), |
| | | 2.76-2.68 (m, 1H), 2.67 (s, 6H), 2.34-2.16 (m, 4H), 2.13-1.71 (m, 9H), 1.39 |
| | | (d, J = 8.8 Hz, 2H) |
| I-133 | 834.8 | 10.94-10.74 (m, 1H), 8.33 (d, J = 4.8 Hz, 1H), 8.12 (s, 1H), 7.70 (s, 1H), 7.44- |
| | | 7.31 (m, 1H), 7.31-7.16 (m, 2H), 6.95-6.82 (m, 2H), 6.81-6.75 (m, 2H), |
| | | 6.35-6.20 (m, 1H), 4.69-4.58 (m, 2H), 4.43-4.30 (m, 2H), 4.01 (d, J = 5.6 |
| | | Hz, 2H), 3.90-3.81 (m, 3H), 3.61 (s, 1H), 3.56 (s, 4H), 3.48 (s, 2H), 3.30 (s, |
| | | 2H), 3.13 (dd, J = 6.4, 16.0 Hz, 3H), 3.04 (s, 3H), 2.92-2.85 (m, 2H), 2.71- |
| | | 2.61 (m, 1H), 2.46 (d, J = 6.4 Hz, 4H), 2.34 (s, 4H), 2.28-2.17 (m, 2H), 2.09- |
| | | 1.98 (m, 1H), 1.89 (d, J = 6.4 Hz, 2H), 1.77 (s, 4H) |
| I-135 | 832.4 | 11.05 (s, 1H), 8.11 (d, J = 1.6 Hz, 1H), 7.69 (d, J = 2.8 Hz, 1H), 7.39-7.30 (m, |
| | | 1H), 7.28-7.19 (m, 1H), 6.94 (d, J = 8.4 Hz, 1H), 6.90 (br d, J = 16.0 Hz, 1H), |
| | | 6.85 (d, J = 2.0 Hz, 1H), 6.63 (dd, J = 2.0, 8.4 Hz, 1H), 6.30-6.21 (m, 1H), |
| | | 5.29 (dd, J = 5.2, 12.8 Hz, 1H), 4.63 (t, J = 6.8 Hz, 2H), 4.35 (br d, J = 14.4 |
| | | Hz, 2H), 3.83 (br d, J = 10.4 Hz, 2H), 3.61 (br t, J = 5.6 Hz, 1H), 3.56 (br t, J = |
| | | 5.6 Hz, 2H), 3.31 (s, 6H), 3.16-3.14 (m, 1H), 3.11 (br s, 2H), 3.10 (br s, 3H), |
| | | 3.03 (br s, 2H), 2.92-2.86 (m, 1H), 2.81 (br t, J = 11.6 Hz, 2H), 2.68 (br dd, J = |
| | | 4.4, 12.8 Hz, 1H), 2.63 (br s, 1H), 2.54 (br s, 4H), 2.33-2.23 (m, 4H), 2.01- |
| | | 1.95 (m, 1H), 1.88 (br d, J = 12.0 Hz, 2H), 1.76 (br d, J = 5.6 Hz, 1H), 1.39- |
| | | 1.29 (m, 2H) |
| I-143 | 854.6 | 11.09 (s, 1H), 8.54-8.49 (m, 1H), 8.23 (s, 1H), 8.17 (s, 1H), 8.15-8.11 (m, |
| | | 1H), 7.79-7.73 (m, 1H), 7.73-7.68 (m, 1H), 7.54-7.45 (m, 2H), 7.09 (s, 1H), |
| | | 7.02 (d, J = 8.0 Hz, 1H), 6.91 (d, J = 8.0 Hz, 1H), 6.34 (br s, 1H), 5.35 (dd, J = |
| | | 5.2, 12.8 Hz, 1H), 4.69-4.61 (m, 2H), 4.49-4.38 (m, 2H), 3.69-3.52 (m, 4H), |
| | | 3.33 (br s, 3H), 3.15 (td, J = 6.8, 17.8 Hz, 5H), 3.06 (br s, 3H), 2.99-2.82 (m, |
| | | 2H), 2.71-2.63 (m, 4H), 2.55 (br d, J = 4.4 Hz, 4H), 2.40-2.36 (m, 4H), 2.29 |
| | | (br s, 1H), 2.07-1.95 (m, 2H), 1.87-1.76 (m, 2H) |
| I-148 | 883.6 | 11.09 (s, 1H), 8.22 (s, 1H), 8.11 (d, J = 2.8 Hz, 1H), 7.77-7.58 (m, 2H), 7.49- |
| | | 7.34 (m, 2H), 7.26 (dd, J = 3.6, 8.4 Hz, 1H), 7.11-6.98 (m, 2H), 6.90 (d, J = |
| | | 8.0 Hz, 1H), 6.72-6.55 (m, 2H), 6.30 (br d, J = 14.0 Hz, 1H), 5.34 (dd, J = |
| | | 5.6, 12.8 Hz, 1H), 4.63 (dt, J = 3.2, 6.8 Hz, 2H), 4.39 (br d, J = 12.0 Hz, 2H), |
| | | 3.71 (d, J = 4.8 Hz, 3H), 3.59 (td, J = 5.6, 18.4 Hz, 3H), 3.29-3.16 (m, 8H), |
| | | 3.15-3.08 (m, 2H), 3.04-2.93 (m, 3H), 2.93-2.84 (m, 1H), 2.73-2.60 (m, |
| | | 4H), 2.53 (br d, J = 6.8 Hz, 4H), 2.41-2.28 (m, 4H), 2.25 (br d, J = 2.4 Hz, |
| | | 1H), 2.05-1.95 (m, 1H), 1.81 (q, J = 7.2 Hz, 2H) |
| I-152 | 867.5 | 11.05 (s, 1H), 8.20 (s, 1H), 8.12 (d, J = 3.6 Hz, 1H), 7.78-7.67 (m, 2H), 7.51- |
| | | 7.33 (m, 3H), 7.08 (s, 1H), 7.00 (d, J = 8.0 Hz, 1H), 6.91 (d, J = 8.4 Hz, 1H), |
| | | 6.35-6.29 (m, 2H), 6.12 (dd, J = 1.6, 8.4 Hz, 1H), 5.27 (dd, J = 5.6, 12.8 Hz, |
| | | 1H), 4.68-4.60 (m, 2H), 4.41 (br d, J = 12.4 Hz, 2H), 3.88 (s, 4H), 3.74 (d, J = |
| | | 4.0 Hz, 2H), 3.65 (s, 2H), 3.58 (br t, J = 5.6 Hz, 2H), 3.41-3.40 (m, 3H), |
| | | 3.29-3.28 (m, 3H), 3.21 (br s, 3H), 3.18-3.09 (m, 4H), 3.04-2.82 (m, 5H), |
| | | 2.69-2.58 (m, 2H), 2.37-2.23 (m, 2H), 2.03-1.92 (m, 1H) |
| I-156 | 442.1^{b} | 11.52-11.41 (m, 1H), 11.08 (s, 1H), 8.24 (s, 1H), 8.14-8.08 (m, 1H), 7.71- |
| | | 7.66 (m, 1H), 7.37-7.30 (m, 1H), 7.19 (d, J = 8.4 Hz, 1H), 7.09-7.00 (m, 3H), |
| | | 6.91 (d, J = 8.0 Hz, 1H), 6.66 (s, 1H), 6.60 (br d, J = 8.4 Hz, 1H), 6.49 (br d, J = |
| | | 2.0 Hz, 1H), 6.24 (br s, 1H), 5.34 (dd, J = 5.2, 12.8 Hz, 1H), 4.66-4.59 (m, |
| | | 2H), 4.42-4.33 (m, 2H), 3.73-3.69 (m, 3H), 3.63-3.57 (m, 2H), 3.25 (br s, |
| | | 12H), 3.14-3.10 (m, 3H), 2.95-2.85 (m, 2H), 2.69 (br s, 2H), 2.65-2.59 (m, |
| | | 2H), 2.54 (br d, J = 0.8 Hz, 3H), 2.40-2.33 (m, 3H), 2.29-2.22 (m, 1H), 2.04- |
| | | 1.98 (m, 1H), 1.86-1.78 (m, 2H) |
| I-157 | 882.6 | 11.43-11.37 (m, 1H), 11.08 (s, 1H), 8.18 (s, 1H), 8.12 (d, J = 1.6 Hz, 1H), 7.69 |
| | | (d, J = 7.6 Hz, 1H), 7.62 (s, 1H), 7.33 (d, J = 4.8 Hz, 1H), 7.19 (dd, J = 4.0, 8.4 |
| | | Hz, 1H), 7.16-7.12 (m, 1H), 7.08 (s, 1H), 7.01 (d, J = 8.0 Hz, 2H), 6.90 (d, J = |
| | | 8.0 Hz, 1H), 6.66 (br s, 1H), 6.60 (br d, J = 8.8 Hz, 1H), 6.47 (dd, J = 1.6, |
| | | 6.8 Hz, 1H), 5.34 (dd, J = 5.2, 12.8 Hz, 1H), 4.65 (dt, J = 2.8, 6.8 Hz, 2H), 3.70 |
| | | (d, J = 8.0 Hz, 3H), 3.63 (br s, 2H), 3.29-3.23 (m, 11H), 3.19 (br d, J = 7.2 |
| | | Hz, 3H), 2.95-2.85 (m, 2H), 2.75-2.68 (m, 2H), 2.65-2.59 (m, 2H), 2.54 (br |
| | | d, J = 4.8 Hz, 3H), 2.37 (br t, J = 7.2 Hz, 3H), 2.04-1.87 (m, 4H), 1.84-1.79 |
| | | (m, 2H) |
| I-158 | 868.7 | 11.52-11.43 (m, 1H), 11.08 (s, 1H), 8.14-8.10 (m, 1H), 7.73-7.64 (m, 1H), |
| | | 7.36-7.32 (m, 1H), 7.20 (d, J = 8.4 Hz, 1H), 7.11 (s, 1H), 7.08-7.00 (m, 2H), |
| | | 6.94 (d, J = 7.2 Hz, 1H), 6.68 (s, 1H), 6.64-6.59 (m, 1H), 6.51-6.47 (m, 1H), |
| | | 6.29-6.22 (m, 1H), 5.34 (dd, J = 5.2, 12.8 Hz, 1H), 4.63 (q, J = 6.4 Hz, 2H), |
| | | 4.43-4.33 (m, 2H), 3.74-3.69 (m, 3H), 3.65-3.56 (m, 2H), 3.34 (s, 3H), 3.26 |
| | | (br s, 6H), 3.17-3.07 (m, 5H), 2.97-2.89 (m, 1H), 2.88-2.82 (m, 2H), 2.71 |
| | | (br dd, J = 4.8, 12.8 Hz, 2H), 2.67-2.58 (m, 7H), 2.35-2.24 (m, 2H), 2.06- |
| | | 1.97 (m, 1H) |
| I-160 | 838.6 | 11.67 (s, 1H), 11.09 (s, 1H), 8.13 (s, 1H), 7.70 (s, 1H), 7.57 (br d, J = 8.8 Hz, |
| | | 2H), 7.38-7.34 (m, 1H), 7.15 (br d, J = 1.2 Hz, 1H), 7.12-7.06 (m, 3H), 7.04- |
| | | 7.00 (m, 1H), 6.93 (d, J = 7.2 Hz, 1H), 6.88-6.85 (m, 1H), 6.32 (br s, 1H), |
| | | 5.34 (dd, J = 5.2, 12.8 Hz, 1H), 4.66-4.61 (m, 2H), 4.44-4.37 (m, 2H), 3.59 |
| | | (s, 2H), 3.33 (br s, 3H), 3.24 (br s, 6H), 3.11 (s, 4H), 2.95-2.89 (m, 1H), 2.82 |
| | | (br d, J = 8.4 Hz, 2H), 2.72 (br s, 1H), 2.69-2.59 (m, 8H), 2.34 (br dd, J = 2.4, |
| | | 4.8 Hz, 2H), 2.29-2.24 (m, 1H), 2.04-1.98 (m, 1H) |
| I-161 | 868.9 | 11.64-11.32 (m, 1H), 8.16-8.05 (m, 1H), 7.72-7.61 (m, 1H), 7.39-7.30 (m, |
| | | 1H), 7.20 (d, J = 8.4 Hz, 1H), 7.08-7.03 (m, 1H), 6.99 (d, J = 5.2 Hz, 2H), |
| | | 6.97-6.93 (m, 1H), 6.69 (s, 1H), 6.61 (br d, J = 8.4 Hz, 1H), 6.51-6.48 (m, |
| | | 1H), 6.25 (br s, 1H), 5.38 (dd, J = 5.2, 12.4 Hz, 1H), 4.66-4.61 (m, 2H), 4.42- |
| | | 4.34 (m, 2H), 3.73-3.70 (m, 3H), 3.62 (s, 4H), 3.58 (br t, J = 5.6 Hz, 1H), |
| | | 3.27 (br s, 6H), 3.16-3.08 (m, 7H), 2.98-2.81 (m, 2H), 2.78-2.71 (m, 1H), |
| | | 2.70-2.63 (m, 7H), 2.34-2.25 (m, 2H), 2.01 (td, J = 5.2, 10.8 Hz, 1H) |
| I-162 | 852.3 | 11.53-11.47 (m, 1H), 10.75 (s, 1H), 8.18-8.10 (m, 2H), 7.71-7.68 (m, 1H), |
| | | 7.26-7.24 (m, 1H), 7.04-6.98 (m, 4H), 6.92 (br s, 1H), 6.39 (d, J = 8.0 Hz, |
| | | 2H), 6.26 (br s, 1H), 4.64 (br t, J = 6.8 Hz, 2H), 4.38 (br s, 1H), 4.34 (br s, 1H), |
| | | 3.95-3.92 (m, 2H), 3.71-3.68 (m, 1H), 3.62 (br t, J = 5.2 Hz, 1H), 3.57 (br t, |
| | | J = 5.6 Hz, 2H), 3.48-3.44 (m, 4H), 3.15 (br s, 1H), 3.13 (br d, J = 4.0 Hz, |
| | | 1H), 3.10 (br s, 1H), 3.09 (br s, 1H), 3.02 (br s, 1H), 2.99 (br s, 2H), 2.97-2.96 |
| | | (m, 1H), 2.95 (br s, 1H), 2.68 (br d, J = 6.4 Hz, 2H), 2.63-2.58 (m, 1H), 2.48- |
| | | 2.46 (m, 1H), 2.45-2.41 (m, 1H), 2.33 (br s, 1H), 2.24 (br d, J = 7.2 Hz, 2H), |
| | | 2.14-2.09 (m, 1H), 2.00 (br dd, J = 4.4, 8.8 Hz, 1H), 1.84 (br d, J = 1.2 Hz, |
| | | 2H) |
| I-163 | 854.6 | 11.59 (s, 1H), 11.13-11.05 (m, 1H), 8.10 (d, J = 12.5 Hz, 1H), 7.69 (d, J = 8.5 |
| | | Hz, 1H), 7.54 (br d, J = 8.5 Hz, 2H), 7.22 (d, J = 10.5 Hz, 1H), 7.08 (s, 2H), |
| | | 7.05 (s, 1H), 7.00 (s, 1H), 6.99-6.94 (m, 1H), 6.93-6.88 (m, 1H), 6.87-6.83 |
| | | (m, 1H), 5.39-5.27 (m, 1H), 4.64-4.57 (m, 2H), 4.57-4.39 (m, 1H), 3.93- |
| | | 3.84 (m, 1H), 3.31-3.29 (m, 6H), 3.22 (br s, 6H), 3.12-3.01 (m, 4H), 3.02- |
| | | 2.82 (m, 2H), 2.78-2.57 (m, 8H), 2.36 (br dd, J = 8.0, 14.5 Hz, 3H), 2.08- |
| | | 1.83 (m, 3H), 1.86-1.70 (m, 4H) |
| I-164 | 871.6 | 11.08 (s, 1H), 8.10 (d, J = 11.6 Hz, 1H), 7.74 (br dd, J = 4.0, 8.8 Hz, 2H), 7.68 |
| | | (d, J = 11.6 Hz, 1H), 7.54 (s, 1H), 7.52-7.44 (m, 1H), 7.09-7.05 (m, 3H), |
| | | 7.01 (d, J = 8.0 Hz, 1H), 6.90 (d, J = 8.0 Hz, 1H), 6.16 (br s, 1H), 5.34 (dd, J = |
| | | 5.2, 12.6 Hz, 1H), 4.62 (q, J = 7.2 Hz, 2H), 4.34 (br d, J = 17.6 Hz, 2H), 3.67- |
| | | 3.59 (m, 2H), 3.31 (s, 3H), 3.27 (br s, 2H), 3.24 (br s, 4H), 3.12-3.07 (m, 2H), |
| | | 3.03 (br s, 3H), 2.94-2.86 (m, 1H), 2.77-2.69 (m, 1H), 2.69-2.64 (m, 3H), |
| | | 2.60 (br s, 1H), 2.54 (br s, 3H), 2.40-2.32 (m, 4H), 2.28 (br s, 1H), 2.03-1.98 |
| | | (m, 1H), 1.85-1.78 (m, 2H) |
| I-165 | 854.9 | 12.17 (br d, J = 1.2 Hz, 1H), 7.43-7.38 (m, 1H), 7.25-7.16 (m, 2H), 7.10- |
| | | 7.02 (m, 1H), 6.97 (dd, J = 2.4, 8.4 Hz, 1H), 6.86 (d, J = 2.8 Hz, 1H), 6.16 (br |
| | | s, 1H), 4.38-4.32 (m, 2H), 3.83 (s, 3H), 3.65-3.58 (m, 2H), 3.25-2.95 (m, |
| | | 6H), 2.40-2.25 (m, 2H), 2.08 (d, J = 6.8 Hz, 3H) |
| I-166 | 884.6 | 11.43 (s, 1H), 11.09 (br d, J = 0.8 Hz, 1H), 8.10 (d, J = 12.8 Hz, 1H), 7.69 (d, J = |
| | | 8.8 Hz, 1H), 7.23-7.16 (m, 2H), 7.09 (s, 1H), 7.02 (d, J = 8.0 Hz, 1H), 6.93- |
| | | 6.86 (m, 2H), 6.68-6.59 (m, 2H), 6.48 (s, 1H), 5.35 (dd, J = 5.2, 12.8 Hz, |
| | | 1H), 4.64-4.58 (m, 2H), 4.52 (br d, J = 9.2 Hz, 1H), 3.87 (br s, 1H), 3.71 (d, J = |
| | | 5.6 Hz, 3H), 3.35 (s, 3H), 3.29 (s, 4H), 3.25 (br s, 4H), 3.16-2.95 (m, 8H), |
| | | 2.70-2.64 (m, 7H), 2.38 (br s, 2H), 2.35-2.32 (m, 2H), 2.05-1.95 (m, 2H), |
| | | 1.85-1.74 (m, 4H) |
| I-169 | 854.6 | 11.50 (br s, 1H), 11.22-10.99 (m, 1H), 8.48 (s, 1H), 8.14-8.10 (m, 1H), 7.71- |
| | | 7.67 (m, 1H), 7.36-7.31 (m, 1H), 7.18 (br d, J = 8.4 Hz, 1H), 7.11 (br d, J = |
| | | 7.6 Hz, 1H), 7.07-7.03 (m, 1H), 7.03-6.92 (m, 2H), 6.66 (br s, 1H), 6.59 (br |
| | | d, J = 8.0 Hz, 1H), 6.52-6.45 (m, 1H), 6.29-6.21 (m, 1H), 5.44-5.35 (m, |
| | | 1H), 4.68-4.59 (m, 2H), 4.45-4.30 (m, 2H), 3.76-3.69 (m, 8H), 3.62-3.56 |
| | | (m, 2H), 3.23 (br s, 5H), 3.11 (br dd, J = 7.6, 14.4 Hz, 5H), 2.95-2.85 (m, 2H), |
| | | 2.71 (br d, J = 4.4 Hz, 1H), 2.66 (br d, J = 1.6 Hz, 1H), 2.60 (br s, 4H), 2.33 (br |
| | | s, 2H), 2.26 (br d, J = 4.4 Hz, 1H), 2.05-2.00 (m, 1H) |
| I-173 | 864.3 | 11.77-11.64 (m, 1H), 11.09 (s, 1H), 8.17 (s, 1H), 8.13-8.10 (m, 1H), 7.71- |
| | | 7.67 (m, 1H), 7.58-7.55 (m, 2H), 7.38-7.34 (m, 1H), 7.18-7.15 (m, 1H), |
| | | 7.08-7.05 (m, 3H), 7.01 (d, J = 8.0 Hz, 1H), 6.90 (d, J = 8.0 Hz, 1H), 6.79 (s, |
| | | 1H), 6.32 (br s, 1H), 5.34 (dd, J = 5.6, 12.8 Hz, 1H), 4.66-4.61 (m, 2H), 4.48 |
| | | (br s, 2H), 4.42-4.37 (m, 2H), 4.08 (br s, 2H), 3.63-3.56 (m, 2H), 3.23 (br s, |
| | | 3H), 3.18-3.14 (m, 1H), 3.13-3.07 (m, 2H), 2.94-2.86 (m, 1H), 2.72 (br d, |
| | | J = 4.4 Hz, 1H), 2.70-2.63 (m, 4H), 2.60 (br s, 1H), 2.54 (br s, 4H), 2.39- |
| | | 2.30 (m, 6H), 2.27 (br d, J = 8.0 Hz, 1H), 2.03-1.97 (m, 1H), 1.85-1.78 (m, |
| | | 2H) |
| I-174 | 420.9 (M + H/2)+ | 11.07 (br d, J = 1.2 Hz, 1H), 7.78 (br d, J = 8.8 Hz, 2H), 7.74-7.68 (m, 1H), |
| | | 7.54-7.47 (m, 2H), 7.41 (s, 2H), 7.12-7.07 (m, 3H), 7.02 (d, J = 8.0 Hz, 1H), |
| | | 6.93 (br d, J = 8.0 Hz, 1H), 6.20 (br d, J = 7.2 Hz, 1H), 5.34 (br dd, J = 4.8, |
| | | 12.8 Hz, 1H), 4.50 (br d, J = 10.4 Hz, 1H), 4.38-4.32 (m, 2H), 3.94-3.83 (m, |
| | | 1H), 3.33 (s, 3H), 3.28 (br s, 3H), 3.26 (br d, J = 3.2 Hz, 4H), 3.20-3.13 (m, |
| | | 1H), 3.03 (br s, 4H), 2.93 (br s, 2H), 2.87-2.80 (m, 3H), 2.72 (br d, J = 4.4 Hz, |
| | | 2H), 2.69-2.66 (m, 2H), 2.63 (br s, 4H), 2.05-1.74 (m, 5H), 1.51 (br s, 1H) |
| I-177 | 819.5 | 11.92 (br s, 1H), 11.06 (s, 1H), 7.20-7.14 (m, 1H), 7.07 (s, 1H), 7.01 (d, J = |
| | | 8.0 Hz, 1H), 6.92-6.80 (m, 2H), 6.65 (s, 1H), 6.59 (br d, J = 8.4 Hz, 1H), 6.48 |
| | | (br s, 1H), 6.14-6.06 (m, 1H), 5.39-5.30 (m, 1H), 4.36-4.28 (m, 2H), 3.71 |
| | | (s, 3H), 3.65-3.57 (m, 2H), 3.34 (s, 3H), 3.28 (s, 3H), 3.24 (br s, 4H), 3.21- |
| | | 3.01 (m, 6H), 2.94-2.86 (m, 1H), 2.70-2.67 (m, 2H), 2.65 (br s, 1H), 2.56- |
| | | 2.55 (m, 2H), 2.36 (br d, J = 6.8 Hz, 3H), 2.30-2.22 (m, 1H), 2.07 (d, J = 8.0 |
| | | Hz, 3H), 2.03-1.96 (m, 1H), 1.85-1.77 (m, 2H) |
| I-179 | 801.5 | 12.19 (s, 1H), 11.09 (s, 1H), 7.56 (d, J = 7.6 Hz, 2H), 7.15-7.07 (m, 3H), 7.06- |
| | | 6.90 (m, 3H), 6.83 (d, J = 2.4 Hz, 1H), 6.15 (s, 1H), 5.35 (dd, J = 5.2, 12.8 |
| | | Hz, 1H), 4.47 (s, 2H), 4.34 (s, 2H), 4.09 (s, 2H), 3.61 (td, J = 5.6, 11.6 Hz, 2H), |
| | | 3.34 (s, 4H), 3.30-2.99 (m, 6H), 2.98-2.83 (m, 2H), 2.78-2.57 (m, 6H), 2.42- |
| | | 2.23 (m, 5H), 2.07 (d, J = 8.8 Hz, 3H), 2.04-1.95 (m, 2H) |
| I-180 | 893.5 | 11.70-11.63 (m, 1H), 11.08 (s, 1H), 8.18 (s, 1H), 8.14-8.10 (m, 1H), 7.73- |
| | | 7.63 (m, 1H), 7.57 (br d, J = 8.4 Hz, 2H), 7.37-7.34 (m, 1H), 7.18-7.14 (m, |
| | | 1H), 7.07 (br d, J = 8.8 Hz, 2H), 6.94 (d, J = 8.4 Hz, 1H), 6.88-6.85 (m, 2H), |
| | | 6.65 (dd, J = 0.8, 9.2 Hz, 1H), 6.32 (br s, 1H), 5.29 (br dd, J = 5.2, 12.8 Hz, |
| | | 1H), 4.66-4.61 (m, 2H), 4.43-4.37 (m, 2H), 3.64 (br d, J = 6.4 Hz, 2H), 3.58 |
| | | (br s, 1H), 3.31 (s, 3H), 3.22 (br s, 7H), 3.15 (br s, 2H), 3.11 (br s, 2H), 2.98- |
| | | 2.83 (m, 2H), 2.73-2.65 (m, 7H), 2.64-2.58 (m, 2H), 2.43-2.37 (m, 1H), |
| | | 2.35-2.25 (m, 2H), 2.01-1.96 (m, 1H), 1.92 (br d, J = 11.2 Hz, 2H), 1.65- |
| | | 1.56 (m, 2H) |
| I-181 | 834.6 | 12.11 (br d, J = 1.2 Hz, 1H), 11.05 (br s, 1H), 8.51 (s, 1H), 8.16-8.06 (m, 1H), |
| | | 7.76-7.62 (m, 1H), 6.99 (br s, 1H), 6.94-6.81 (m, 2H), 6.45 (br s, 1H), 6.33 |
| | | (br d, J = 8.0 Hz, 1H), 6.08 (br s, 1H), 5.72-5.49 (m, 1H), 5.25 (br dd, J = 4.0, |
| | | 11.6 Hz, 1H), 4.63 (br s, 2H), 4.35-4.27 (m, 2H), 4.24-4.03 (m, 1H), 3.99- |
| | | 3.59 (m, 8H), 3.49 (br d, J = 5.6 Hz, 2H), 3.27 (br s, 3H), 3.19 (br s, 5H), 3.10 |
| | | (br d, J = 7.2 Hz, 2H), 2.89 (br s, 2H), 2.65 (br d, J = 16.8 Hz, 2H), 2.39-2.24 |
| | | (m, 5H), 2.08 (br d, J = 0.8 Hz, 4H), 2.00-1.92 (m, 2H) |
| I-183 | 817.4 | 12.14 (br d, J = 3.2 Hz, 1H), 11.16-11.03 (m, 1H), 7.62-7.44 (m, 2H), 7.11- |
| | | 6.85 (m, 7H), 6.23-6.07 (m, 1H), 5.46-5.27 (m, 1H), 4.42-4.32 (m, 2H), |
| | | 3.67 (br d, J = 3.2 Hz, 2H), 3.29-3.14 (m, 8H), 3.13-2.77 (m, 6H), 2.75-2.54 |
| | | (m, 8H), 2.45-2.32 (m, 4H), 2.31-2.14 (m, 1H), 2.05-1.96 (m, 1H), 1.82 (br |
| | | d, J = 4.4 Hz, 2H), 1.07-0.98 (m, 6H) |
| I-185 | 887.6 | 11.99 (s, 1H), 11.09 (s, 1H), 7.17 (d. J = 8.4 Hz, 1H), 7.08 (s, 1H), 7.02 (d, J = |
| | | 8.0 Hz, 1H), 6.93-6.82 (m, 2H), 6.66 (d, J = 1.6 Hz, 1H), 6.59 (d, J = 8.4 Hz, |
| | | 1H), 6.37 (s, 1H), 6.10 (s, 1H), 5.35 (dd, J = 5.2, 12.7 Hz, 1H), 4.41-4.31 (m, |
| | | 2H), 3.71 (s, 3H), 3.69-3.59 (m, 6H), 3.34 (s, 3H), 3.24 (s, 4H), 3.00-2.85 |
| | | (m, 2H), 2.79-2.63 (m, 4H), 2.57-2.52 (m, 4H), 2.41-2.26 (m, 4H), 2.06- |
| | | 1.97 (m, 1H), 1.87-1.77 (m, 2H), 1.63 (d, J = 4.4 Hz, 2H), 1.53 (s, 4H), 1.08- |
| | | 0.97 (m, 6H) |
| I-190 | 872.7 | 12.02 (s, 1H), 11.09 (s, 1H), 8.11 (d, J = 11.2 Hz, 1H), 7.72-7.67 (m, 1H), 7.51 |
| | | (d, J = 8.8 Hz, 2H), 7.09-6.98 (m, 5H), 6.91 (dd, J = 1.2, 8.0 Hz, 1H), 6.84 (s, |
| | | 1H), 5.35 (dd, J = 5.6, 12.8 Hz, 1H), 4.62 (q, J = 6.8 Hz, 2H), 4.49 (d, J = 11.6 |
| | | Hz, 1H), 3.91 (d, J = 12.0 Hz, 1H), 3.32 (s, 3H), 3.22 (s, 6H), 3.13-2.97 (m, |
| | | 7H), 2.94-2.76 (m, 2H), 2.72-2.63 (m, 4H), 2.54 (d, J = 5.6 Hz, 4H), 2.37 (t, |
| | | J = 7.2 Hz, 2H), 2.04-1.71 (m, 7H), 1.61-1.39 (m, 1H) |
| I-189 | 872.8 | 12.10-11.98 (m, 1H), 11.09 (s, 1H), 8.11 (d, J = 11.6 Hz, 1H), 7.69 (d, J = |
| | | 10.4 Hz, 1H), 7.51 (d, J = 8.8 Hz, 2H), 7.09-6.99 (m, 5H), 6.91 (d, J = 8.0 Hz, |
| | | 1H), 6.84 (s, 1H), 5.35 (dd, J = 5.6, 12.8 Hz, 1H), 4.62 (q, J = 6.8 Hz, 2H), 4.49 |
| | | (d, J = 12.0 Hz, 1H), 3.91 (d, J = 12.0 Hz, 1H), 3.34 (s, 3H), 3.22 (s, 6H), 3.13- |
| | | 2.99 (m, 7H), 2.94-2.79 (m, 2H), 2.70-2.63 (m, 4H), 2.54 (d, J = 4.4 Hz, |
| | | 4H), 2.37 (t, J = 7.2 Hz, 2H), 2.04-1.76 (m, 7H), 1.60-1.38 (m, 1H) |
| I-191 | 896.4 | 12.12 (d, J = 8.8 Hz, 1H), 10.79 (s, 1H), 7.75-7.68 (m, 1H), 7.46-7.34 (m, |
| | | 2H), 7.02 (t, J = 8.4 Hz, 1H), 6.98-6.86 (m, 3H), 6.62 (br s, 1H), 6.51- 6.43 |
| | | (m, 2H), 6.22-6.16 (m, 1H), 6.15-6.09 (m, 1H), 6.03 (d, J = 7.6 Hz, 1H), 4.40- |
| | | 4.27 (m, 5H), 3.93 (d, J = 12.0 Hz, 2H), 3.67-3.56 (m, 2H), 3.26-2.91 (m, |
| | | 9H), 2.86 (t, J = 10.4 Hz, 3H), 2.79-2.68 (m, 1H), 2.61-2.54 (m, 1H), 2.35- |
| | | 2.24 (m, 2H), 2.13-2.05 (m, 1H), 1.91-1.70 (m, 5H) |
| I-192 | 861.4 | (chloroform-d) 9.42-9.22 (m, 1H), 8.16-8.03 (m, 1H), 7.77 (d, J = 5.2 Hz, |
| | | 1H), 7.67 (d, J = 4.4 Hz, 1H), 6.70 (s, 2H), 6.67 (br s, 2H), 6.34 (dd, J = 5.2, |
| | | 12.8 Hz, 1H), 6.13-6.06 (m, 1H), 5.20 (dd, J = 5.2, 12.8 Hz, 1H), 4.80 (q, J = |
| | | 6.0 Hz, 2H), 4.59 (br d, J = 5.2 Hz, 2H), 4.43 (br s, 1H), 4.29 (br s, 2H), 4.25 |
| | | (br s, 1H), 3.77 (t, J = 5.6 Hz, 1H), 3.61 (br s, 1H), 3.60-3.56 (m, 2H), 3.42 |
| | | (s, 3H), 3.19 (br d, J = 1.2 Hz, 4H), 3.09-3.03 (m, 2H), 2.99-2.92 (m, 1H), |
| | | 2.88-2.80 (m, 1H), 2.77-2.68 (m, 4H), 2.66 (br d, J = 4.0 Hz, 2H), 2.50-2.44 |
| | | (m, 2H), 2.36 (br s, 4H), 2.27-2.21 (m, 1H), 2.02-1.91 (m, 2H), 1.83-1.71 |
| | | (m, 1H), 1.68 (br d, J = 2.0 Hz, 1H), 1.56-1.46 (m, 2H). |
| I-196 | 847.6 | 11.95 (s, 1H), 11.09 (s, 1H), 8.34 (s, 1H), 7.17 (d, J = 8.4 Hz, 1H), 7.08 (s, 1H), |
| | | 7.01 (d, J = 8.0 Hz, 1H), 6.90 (d, J = 7.2 Hz, 1H), 6.88-6.81 (m, 1H), 6.65 (s, |
| | | 1H), 6.59 (d, J = 9.2 Hz, 1H), 6.48 (s, 1H), 6.09 (s, 1H), 5.34 (dd, J = 5.2, 12.8 |
| | | Hz, 1H), 4.38-4.32 (m, 2H), 3.71 (s, 3H), 3.66 (d, J = 5.2 Hz, 2H), 3.24 (s, |
| | | 8H), 3.18 (s, 3H), 3.06 (s, 3H), 2.98-2.84 (m, 3H), 2.70-2.62 (m, 4H), 2.39- |
| | | 2.34 (m, 4H), 2.33 (dd, J = 1.6, 3.6 Hz, 1H), 2.26 (dd, J = 1.2, 7.2 Hz, 1H), |
| | | 2.04-1.97 (m, 1H), 1.85-1.77 (m, 2H), 1.05-0.99 (m, 6H) |
| I-216 | 838.8 | 11.81-11.50 (m, 1H), 11.09 (s, 1H), 8.18-8.07 (m, 1H), 7.74-7.65 (m, 1H), |
| | | 7.57 (d, J = 8.8 Hz, 2H), 7.43-7.31 (m, 1H), 7.22-7.13 (m, 1H), 7.08 (d, J = |
| | | 8.4 Hz, 2H), 7.03-6.91 (m, 3H), 6.87 (s, 1H), 6.32 (s, 1H), 5.44-5.30 (m, 1H), |
| | | 4.76-4.55 (m, 2H), 4.50-4.26 (m, 2H), 3.75-3.49 (m, 5H), 3.25 (s, 6H), 3.18- |
| | | 2.99 (m, 7H), 2.96-2.84 (m, 1H), 2.79-2.54 (m, 9H), 2.35-2.24 (m, 2H), |
| | | 2.00 (dd, J = 5.2, 10.4 Hz, 1H) |
| I-217 | 837.4 | 11.77-11.49 (m, 1H), 11.10 (s, 1H), 7.77-7.67 (m, 1H), 7.57 (d, J = 8.8 Hz, |
| | | 2H), 7.47-7.39 (m, 1H), 7.38-7.31 (m, 1H), 7.21-7.13 (m, 1H), 7.08 (d, J = |
| | | 8.8 Hz, 2H), 7.02-6.90 (m, 3H), 6.87 (d, J = 2.0 Hz, 1H), 6.32 (s, 1H), 6.24- |
| | | 6.16 (m, 1H), 5.38 (dd, J = 5.6, 12.8 Hz, 1H), 4.55-4.17 (m, 4H), 3.62 (s, 3H), |
| | | 3.56 (t, J = 5.6 Hz, 2H), 3.25 (s, 6H), 3.19-3.09 (m, 4H), 3.01 (td, J = 6.8, 13.2 |
| | | Hz, 3H), 2.95-2.83 (m, 1H), 2.80-2.71 (m, 1H), 2.70-2.63 (m, 6H), 2.63- |
| | | 2.53 (m, 2H), 2.36-2.22 (m, 2H), 2.06-1.93 (m, 1H) |
| I-221 | 875.0 | 11.55-11.41 (m, 1H), 11.08 (s, 1H), 8.19 (s, 1H), 7.70-7.63 (m, 1H), 7.57- |
| | | 7.52 (m, 1H), 7.26-7.19 (m, 1H), 7.05 (s, 1H), 7.03-6.96 (m, 2H), 6.92 (s, |
| | | 1H), 6.87 (d, J = 8.0 Hz, 1H), 6.24 (s, 1H), 5.33 (dd, J = 5.2, 12.8 Hz, 1H), |
| | | 4.36 (br s, 2H), 3.63-3.59 (m, 2H), 3.33 (s, 3H), 3.26 (t, J = 6.8 Hz, 4H), 3.17 |
| | | (d, J = 6.4 Hz, 4H), 2.97-2.87 (m, 4H), 2.70 (dd, J = 4.0, 13.2 Hz, 1H), 2.66- |
| | | 2.54 (m, 7H), 2.45-2.36 (m, 4H), 2.36-2.20 (m, 5H), 2.05-1.88 (m, 5H), |
| | | 1.80-1.70 (m, 2H), 1.69-1.56 (m, 2H), 1.47 (q, J = 11.2 Hz, 2H) |
| I-222 | 874.7 | 11.54-11.41 (m, 1H), 11.07 (s, 1H), 8.19 (s, 1H), 7.71-7.61 (m, 1H), 7.57- |
| | | 7.50 (m, 1H), 7.29-7.19 (m, 1H), 7.06 (s, 1H), 7.03-6.94 (m, 3H), 6.88 (d, J = |
| | | 8.0 Hz, 1H), 6.21 (s, 1H), 5.33 (dd, J = 5.2, 12.4 Hz, 1H), 4.37 (s, 2H), 3.63- |
| | | 3.59 (m, 2H), 3.32 (s, 3H), 3.26 (t, J = 7.2 Hz, 4H), 3.21-3.08 (m, 4H), 3.02- |
| | | 2.84 (m, 5H), 2.77-2.56 (m, 6H), 2.45-2.43 (m, 2H), 2.33 (s, 5H), 2.22 (s, |
| | | 2H), 2.17-1.93 (m, 6H), 1.78 (s, 2H), 1.60-1.48 (m, 4H) |
| I-224 | 760.6 | 12.18 (s, 1H), 11.10 (s, 1H), 8.16 (s, 1H), 7.63-7.55 (m, 2H), 7.37 (d, J = 8.0 |
| | | Hz, 2H), 7.15 (s, 1H), 7.08-6.97 (m, 3H), 6.86 (d, J = 2.4 Hz, 1H), 6.16 (d, |
| | | J = 1.6 Hz, 1H), 5.37 (dd, J = 5.6, 12.8 Hz, 1H), 4.34 (d, J = 7.2 Hz, 2H), 3.64- |
| | | 3.58 (m, 2H), 3.55 (s, 2H), 3.36 (s, 6H), 3.02-2.82 (m, 4H), 2.79-2.56 (m, |
| | | 5H), 2.38-2.25 (m, 2H), 2.14-1.99 (m, 6H), 1.83-1.63 (m, 4H) |
| I-225 | 829.6 | 11.53 (s, 1H), 11.08 (s, 1H), 8.26-8.04 (m, 1H), 7.73-7.61 (m, 1H), 7.34 (s, |
| | | 1H), 7.14 (d, J = 8.4 Hz, 1H), 7.08 (s, 1H), 7.01 (d, J = 8.0 Hz, 1H), 6.90 (d, |
| | | J = 8.0 Hz, 1H), 6.81-6.83 (m, 1H), 6.65 (s, 1H), 6.58 (d, J = 8.8 Hz, 1H), 6.18 |
| | | (s, 1H), 6.07 (d, J = 10.8 Hz, 1H), 5.34-5.36 (m, 1H), 4.62-4.63 (m, 2H), |
| | | 4.40-4.26 (m, 2H), 3.73-3.67 (m, 3H), 3.65-3.58 (m, 2H), 3.34 (s, 3H), 3.23 |
| | | (s, 4H), 3.13-3.07 (m, 2H), 2.94-2.85 (m, 1H), 2.76 (s, 4H), 2.61-2.53 (m, |
| | | 4H), 2.40-2.25 (m, 4H), 2.06-1.96 (m, 1H), 1.89-1.72 (m, 2H) |
| I-226 | 805.5 | 11.94 (s, 1H), 11.08 (s, 1H), 8.18 (s, 1H), 7.17 (d, J = 8.0 Hz, 1H), 7.11 (s, 1H), |
| | | 7.02 (d, J = 8.0 Hz, 1H), 6.93 (d, J = 8.0 Hz, 1H), 6.90-6.80 (m, 1H), 6.67 (s, |
| | | 1H), 6.60 (d, J = 7.6 Hz, 1H), 6.48 (s, 1H), 6.10 (s, 1H), 5.43-5.27 (m, 1H), |
| | | 4.38-4.24 (m, 2H), 3.71 (s, 3H), 3.67-3.52 (m, 4H), 3.50-3.41 (m, 2H), 3.34 |
| | | (s, 3H), 3.21-3.00 (m, 6H), 2.99-2.78 (m, 4H), 2.68 (s, 2H), 2.64 (s, 3H), |
| | | 2.62-2.60 (m, 2H), 2.37 (s, 1H), 2.26 (s, 1H), 2.08 (s, 2H), 2.07-1.93 (m, 2H) |
| I-229 | 755.3 | 12.01 (br s, 1H), 10.90 (s, 1H), 7.41 (s, 1H), 7.33-7.26 (m, 2H), 7.04-6.89 |
| | | (m, 4H), 6.47 (br s, 1H), 6.12 (br s, 1H), 4.39-4.29 (m, 2H), 4.24-4.15 (m, |
| | | 1H), 3.70-3.57 (m, 6H), 3.52 (br s, 2H), 3.30 (br s, 2H), 3.20-3.05 (m, 9H), |
| | | 2.84-2.72 (m, 2H), 2.39-2.25 (m, 5H), 2.08 (br d, J = 7.2 Hz, 3H), 1.99 (br |
| | | d, J = 8.8 Hz, 1H) |
| I-230 | 819.7 | 12.00 (s, 1H), 11.07 (s, 1H), 7.07-6.88 (m, 7H), 6.47 (s, 1H), 6.20-6.09 (m, |
| | | 1H), 5.34 (dd, J = 5.2, 12.6 Hz, 1H), 4.39-4.29 (m, 2H), 3.67-3.58 (m, 5H), |
| | | 3.33 (s, 3H), 3.29 (s, 2H), 3.19-3.03 (m, 9H), 2.95-2.85 (m, 1H), 2.76-2.59 |
| | | (m, 6H), 2.42-2.23 (m, 5H), 2.08 (d, J = 7.2 Hz, 3H), 2.04-1.96 (m, 1H), |
| | | 1.83-1.73 (m, 2H) |
| I-232 | 856.6 | 12.14 (br d, J = 6.8 Hz, 1H), 11.11 (s, 1H), 8.35 (s, 1H), 8.16-8.09 (m, 1H), |
| | | 7.74-7.65 (m, 1H), 7.55-7.51 (m, 2H), 7.07 (br d, J = 8.8 Hz, 2H), 7.01-6.98 |
| | | (m, 2H), 6.97-6.92 (m, 2H), 6.86 (br s, 1H), 6.15 (br s, 1H), 5.38 (br dd, J = |
| | | 5.6, 12.8 Hz, 1H), 4.66-4.60 (m, 2H), 4.40-4.32 (m, 2H), 3.66 (br t, J = 5.6 |
| | | Hz, 1H), 3.61-3.58 (m, 1H), 3.25 (br s, 4H), 3.19-3.05 (m, 9H), 3.04-2.99 |
| | | (m, 1H), 2.92-2.86 (m, 1H), 2.78-2.72 (m, 1H), 2.72-2.62 (m, 8H), 2.61 (br |
| | | s, 1H), 2.36 (br d, J = 2.8 Hz, 2H), 2.31-2.25 (m, 1H), 2.03-1.97 (m, 1H) |
| I-233 | 725.4 | 12.33-12.04 (m, 1H), 10.79 (s, 1H), 7.42 (s, 1H), 7.37-7.26 (m, 3H), 7.13 (s, |
| | | 1H), 7.08-6.94 (m, 3H), 6.87-6.80 (m, 1H), 6.19-6.11 (m, 1H), 4.38-4.30 |
| | | (m, 2H), 4.23-4.15 (m, 1H), 3.64-3.57 (m, 2H), 3.57-3.51 (m, 2H), 3.22 (s, |
| | | 4H), 3.08-3.01 (m, 2H), 2.81-2.73 (m, 1H), 2.55 (s, 4H), 2.37 (d, J = 2.8 Hz, |
| | | 1H), 2.34-2.23 (m, 2H), 2.07 (d, J = 8.4 Hz, 3H), 2.03-1.96 (m, 1H) |
| I-234 | 775.8 | 12.15-12.11 (m, 1H), 11.09 (br s, 1H), 8.42 (s, 1H), 7.56-7.50 (m, 2H), 7.11 |
| | | (s, 1H), 7.07 (br d, J = 8.8 Hz, 2H), 7.03 (d, J = 8.0 Hz, 1H), 6.95-6.93 (m, |
| | | 1H), 6.86 (br s, 1H), 6.15 (br s, 1H), 5.38-5.32 (m, 1H), 4.37-4.32 (m, 2H), |
| | | 3.62 (br dd, J = 5.6, 11.6 Hz, 3H), 3.24 (br s, 6H), 2.85-2.80 (m, 4H), 2.64 (br |
| | | s, 8H), 2.59 (br s, 3H), 2.38 (br dd, J = 5.2, 8.4 Hz, 3H), 2.30-2.26 (m, 1H), |
| | | 2.08 (d, J = 7.6 Hz, 3H), 2.02 (br d, J = 5.6 Hz, 1H) |
| I-235 | 831.5 | 11.90-11.77 (m, 1H), 11.09 (s, 1H), 7.16-7.18 (m, 1H), 7.11 (s, 1H), 7.02 (d, |
| | | J = 8.0 Hz, 1H), 6.93 (d, J = 8.0 Hz, 1H), 6.89-6.78 (m, 1H), 6.75-6.68 (m, |
| | | 1H), 6.65 (s, 1H), 6.59 (d, J = 8.4 Hz, 1H), 6.10 (d, J = 9.6 Hz, 1H), 5.34-5.36 |
| | | (m 1H), 4.37-4.26 (m, 2H), 3.70 (s, 3H), 3.60-3.63 (m, 2H), 3.33 (s, 3H), |
| | | 3.24 (s, 4H), 3.20-3.04 (m, 2H), 3.02 (s, 3H), 2.93 (s, 1H), 2.86-2.79 (m, |
| | | 2H), 2.75-2.66 (m, 2H), 2.62 (s, 3H), 2.59 (s, 2H), 2.36 (s, 1H), 2.25 (s, 1H), |
| | | 2.07 (d, J = 7.6 Hz, 3H), 2.02-1.96 (m, 1H), 0.72 (d, J = 6.4 Hz, 2H), 0.60 (s, |
| | | 2H) |
| I-237 | 801.5 | 12.19-11.89 (m, 1H), 11.12 (s, 1H), 11.08-10.07 (m, 1H), 7.68-7.54 (m, |
| | | 2H), 7.17 (d, J = 8.8 Hz, 2H), 7.13-7.03 (m, 3H), 7.03-6.92 (m, 2H), 6.17 (d, |
| | | J = 4.0 Hz, 1H), 5.58-5.28 (m, 1H), 4.36 (s, 2H), 3.99 (d, J = 9.6 Hz, 2H), |
| | | 3.77 (d, J = 11.6 Hz, 2H), 3.67 (s, 3H), 3.65-3.60 (m, 2H), 3.46 (s, 2H), 3.32- |
| | | 3.19 (m, 4H), 3.17-3.13 (m, 1H), 3.06 (s, 3H), 2.98-2.85 (m, 1H), 2.81- |
| | | 2.61 (m, 4H), 2.40 (s, 1H), 2.29 (s, 1H), 2.09 (d, J = 7.6 Hz, 3H), 2.05-1.94 |
| | | (m, 1H), 0.76 (d, J = 6.8 Hz, 2H), 0.69-0.55 (m, 2H) |
| I-238 | 856.5 | 12.19-12.13 (m, 1H), 11.09-11.06 (m, 1H), 8.13-8.08 (m, 1H), 7.72-7.65 |
| | | (m, 1H), 7.35-7.30 (m, 1H), 7.15-7.13 (m, 1H), 7.12-7.03 (m, 3H), 7.00 (s, |
| | | 3H), 6.94-6.90 (m, 1H), 6.85-6.82 (m, 1H), 6.18-6.13 (m, 1H), 5.36-5.31 |
| | | (m, 1H), 4.62 (br d, J = 7.6 Hz, 2H), 4.40-4.32 (m, 2H), 3.67-3.59 (m, 3H), |
| | | 3.26-3.20 (m, 6H), 3.14-3.06 (m, 5H), 2.94-2.79 (m, 4H), 2.76-2.67 (m, |
| | | 2H), 2.62 (br s, 7H), 2.36 (br s, 2H), 2.04-1.97 (m, 1H) |
| I-240 | 856.5 | 12.35-12.00 (m, 1H), 11.34-10.93 (m, 1H), 8.20-8.02 (m, 1H), 7.75-7.63 |
| | | (m, 1H), 7.52 (br d, J = 8.4 Hz, 2H), 7.11 (s, 1H), 7.06 (br d, J = 8.8 Hz, 2H), |
| | | 7.03-6.92 (m, 3H), 6.85 (br s, 1H), 6.15 (br d, J = 6.4 Hz, 1H), 5.34 (dd, J = |
| | | 5.2, 12.8 Hz, 1H), 4.62 (q, J = 7.2 Hz, 2H), 4.39-4.31 (m, 2H), 3.67-3.58 (m, |
| | | 2H), 3.23 (br s, 6H), 3.15-3.03 (m, 6H), 2.95-2.75 (m, 4H), 2.69-2.58 (m, |
| | | 9H), 2.34 (br dd, J = 1.6, 8.8 Hz, 2H), 2.28 (br s, 1H), 2.04-1.98 (m, 1H) |
| I-243 | 428.3^{b} | 12.12 (br s, 1H), 11.08 (br s, 1H), 7.83-7.63 (m, 1H), 7.53 (br d, J = 8.4 Hz, |
| | | 2H), 7.47-7.35 (m, 1H), 7.12 (s, 1H), 7.07 (br d, J = 8.8 Hz, 2H), 7.03 (d, J = |
| | | 8.0 Hz, 1H), 7.00-6.91 (m, 2H), 6.86 (d, J = 2.8 Hz, 1H), 6.27-6.17 (m, 1H), |
| | | 6.14 (br s, 1H), 5.35 (dd, J = 5.2, 12.8 Hz, 1H), 4.41-4.24 (m, 4H), 3.70-3.61 |
| | | (m, 1H), 3.59 (br t, J = 5.6 Hz, 1H), 3.34 (s, 3H), 3.24 (br s, 4H), 3.19-3.04 |
| | | (m, 4H), 3.02-2.91 (m, 3H), 2.90-2.78 (m, 3H), 2.77-2.67 (m, 2H), 2.66- |
| | | 2.59 (m, 7H), 2.40-2.23 (m, 2H), 2.07-1.97 (m, 1H) |
| I-247 | 805.4 | 11.94 (s, 1H), 11.10 (s, 1H), 8.22 (s, 1H), 7.19 (dd, J = 2.8, 8.0 Hz, 1H), 7.03- |
| | | 6.92 (m, 3H), 6.90-6.81 (m, 1H), 6.68 (s, 1H), 6.61 (d, J = 8.0 Hz, 1H), 6.49 |
| | | (s, 1H), 6.16-6.06 (m, 1H), 5.38 (dd, J = 5.2, 12.8 Hz, 1H), 4.44-4.22 (m, |
| | | 2H), 3.72 (s, 3H), 3.63 (s, 3H), 3.62-3.56 (m, 2H), 3.27 (s, 4H), 3.18-3.05 |
| | | (m, 6H), 2.90 (s, 1H), 2.78-2.70 (m, 2H), 2.68 (s, 3H), 2.67-2.59 (m, 4H), |
| | | 2.37 (s, 2H), 2.31-2.23 (m, 1H), 2.08 (d, J = 7.2 Hz, 3H), 2.02 (dd, J = 5.6, |
| | | 11.2 Hz, 1H) |
| I-250 | 857.7 | 11.57-11.37 (m, 1H), 11.16-10.98 (m, 1H), 7.72 (s, 1H), 7.46-7.38 (m, 1H), |
| | | 7.23 (s, 1H), 7.06 (s, 1H), 7.03-6.94 (m, 3H), 6.88 (d, J = 8.0 Hz, 1H), 6.20 |
| | | (s, 2H), 5.33 (dd, J = 4.8, 12.0 Hz, 1H), 4.42-4.26 (m, 4H), 3.63-3.52 (m, |
| | | 2H), 3.29 (s, 4H), 3.24-3.07 (m, 4H), 3.04-2.93 (m, 4H), 2.92-2.84 (m, 1H), |
| | | 2.31 (s, 5H), 2.26-2.18 (m, 2H), 2.17-1.95 (m, 6H), 1.83-1.71 (m, 2H), 1.54 |
| | | (s, 4H) |
| I-252 | 857.9 | 11.53-11.38 (m, 1H), 11.07 (s, 1H), 7.73 (d, J = 2.0 Hz, 1H), 7.45-7.39 (m, |
| | | 1H), 7.22 (s, 1H), 7.05 (s, 1H), 7.02-6.97 (m, 2H), 6.92 (s, 1H), 6.87 (d, J = |
| | | 8.0 Hz, 1H), 6.27-6.16 (m, 2H), 5.33 (dd, J = 5.6, 12.4 Hz, 1H), 4.41- 4.27 |
| | | (m, 4H), 3.63-3.52 (m, 2H), 3.33-3.32 (m, 3H), 3.30 (s, 3H), 3.22-3.10 (m, |
| | | 3H), 3.05-2.93 (m, 3H), 2.93-2.81 (m, 2H), 2.74-2.67 (m, 1H), 2.65-2.59 |
| | | (m, 3H), 2.56 (s, 2H), 2.45-2.33 (m, 5H), 2.33-2.20 (m, 5H), 2.03-1.89 (m, |
| | | 5H), 1.74 (q, J = 7.2 Hz, 2H), 1.68-1.57 (m, 2H), 1.54-1.40 (m, 2H) |
| I-253 | 857.8 | 11.54-11.37 (m, 1H), 11.14-11.01 (m, 1H), 7.72 (s, 1H), 7.45-7.38 (m, 1H), |
| | | 7.22 (s, 1H), 7.04-6.83 (m, 5H), 6.28-6.14 (m, 2H), 5.36 (dd, J = 5.2, 12.8 |
| | | Hz, 1H), 4.41-4.26 (m, 4H), 3.62-3.52 (m, 5H), 3.29 (s, 3H), 3.23-3.11 (m, |
| | | 3H), 3.00-2.89 (m, 5H), 2.74-2.62 (m, 3H), 2.61-2.59 (m, 1H), 2.55 (s, 2H), |
| | | 2.42-2.29 (m, 10H), 2.01-1.89 (m, 5H), 1.79-1.71 (m, 2H), 1.69-1.58 (m, |
| | | 2H), 1.52-1.41 (m, 2H) |
| I-254 | 857.6 | 11.53-11.42 (m, 1H), 11.09 (s, 1H), 7.76-7.70 (m, 1H), 7.45-7.39 (m, 1H), |
| | | 7.24 (s, 1H), 7.01-6.94 (m, 4H), 6.93-6.88 (m, 1H), 6.21 (s, 2H), 5.37 (dd, |
| | | J = 5.2, 12.4 Hz, 1H), 4.42-4.25 (m, 4H), 3.64-3.53 (m, 5H), 3.32-3.07 (m, |
| | | 6H), 3.06-2.83 (m, 7H), 2.77-2.69 (m, 1H), 2.63 (d, J = 18.0 Hz, 2H), 2.47 |
| | | (d, J = 3.2 Hz, 4H), 2.39 (d, J = 6.0 Hz, 3H), 2.31 (s, 2H), 2.22 (s, 2H), 2.13- |
| | | 1.97 (m, 5H), 1.85-1.73 (m, 2H), 1.62-1.49 (m, 4H) |
| I-259 | 873.6 | 12.11 (d, J = 7.2 Hz, 1H), 11.10 (s, 1H), 7.76-7.67 (m, 1H), 7.52 (d, J = 8.0 |
| | | Hz, 2H), 7.45-7.35 (m, 1H), 7.06 (d, J = 8.4 Hz, 2H), 7.04-6.83 (m, 4H), |
| | | 6.24-6.16 (m, 1H), 6.13 (s, 1H), 5.38 (dd, J = 5.6, 12.4 Hz, 1H), 4.36 (q, J = |
| | | 7.2 Hz, 3H), 4.30 (s, 1H), 3.69-3.53 (m, 5H), 3.24 (s, 5H), 3.12 (d, J = 7.2 Hz, |
| | | 7H), 3.00-2.94 (m, 2H), 2.92-2.71 (m, 2H), 2.70-2.59 (m, 7H), 2.33 (s, 2H), |
| | | 2.05-1.94 (m, 1H) |
| I-260 | 874.5 | 12.17 (d, J = 6.4 Hz, 1H), 11.10 (s, 1H), 8.14-8.09 (m, 1H), 7.73-7.63 (m, |
| | | 1H), 7.38-7.29 (m, 1H), 7.15 (s, 1H), 7.09-6.97 (m, 4H), 6.93-6.87 (m, 1H), |
| | | 6.83 (s, 1H), 6.16 (s, 1H), 5.37 (dd, J = 5.2, 12.4 Hz, 1H), 4.67-4.57 (m, 2H), |
| | | 4.42-4.30 (m, 2H), 3.69-3.56 (m, 5H), 3.24 (s, 6H), 3.15-3.07 (m, 6H), 2.78 |
| | | (s, 2H), 2.72-2.59 (m, 8H), 2.39-2.31 (m, 3H), 2.04-1.94 (m, 1H) |
| I-261 | 860.5 | 12.16 (d, J = 3.6 Hz, 1H), 11.26-10.98 (m, 1H), 8.20-8.00 (m, 1H), 7.76- |
| | | 7.54 (m, 1H), 7.32-7.36 (m, 1H), 7.15-7.08 (m, 2H), 7.04 (d, J = 6.4 Hz, 1H), |
| | | 7.03-6.86 (m, 3H), 6.81 (d, J = 2.4 Hz, 1H), 6.15 (s, 1H), 5.39-5.42 (m, 1H), |
| | | 4.69-4.54 (m, 2H), 4.41-4.25 (m, 2H), 3.76 (s, 2H), 3.71 (s, 3H), 3.67-3.62 |
| | | (m, 1H), 3.61-3.57 (m, 1H), 3.25-3.19 (m, 4H), 3.18-3.04 (m, 6H), 3.02- |
| | | 2.81 (m, 2H), 2.75-2.67 (m, 1H), 2.62 (s, 3H), 2.58 (s, 2H), 2.44-2.31 (m, |
| | | 2H), 2.30-2.21 (m, 1H), 2.09-1.96 (m, 1H) |
| I-262 | 809.8 | 12.07-11.80 (m, 1H), 11.26-11.01 (m, 1H), 7.20-7.08 (m, 2H), 6.92-6.96 |
| | | (m, 1H), 6.89-6.80 (m, 1H), 6.65 (s, 1H), 6.57 (d, J = 8.4 Hz, 1H), 6.47 (s, |
| | | 1H), 6.18-6.05 (m, 1H), 5.40-5.46 (m, 1H), 4.42-4.22 (m, 2H), 3.77 (s, 2H), |
| | | 3.73 (s, 3H), 3.69 (s, 3H), 3.60-3.63 (m, 2H), 3.22 (s, 4H), 3.20-2.94 (m, |
| | | 6H), 2.94-2.82 (m, 1H), 2.79-2.68 (m, 1H), 2.63 (s, 3H), 2.58 (s, 2H), 2.40- |
| | | 2.21 (m, 2H), 2.07 (d, J = 8.0 Hz, 3H), 2.03 (s, 1H) |
| I-264 | 848.4 | 12.83-12.45 (m, 1H), 11.07 (s, 1H), 11.05-10.87 (m, 1H), 7.75 (s, 1H), 7.70- |
| | | 7.48 (m, 1H), 7.46-7.41 (m, 1H), 7.03 (d, J = 8.4 Hz, 1H), 6.97 (s, 1H), 6.75 |
| | | (d, J = 8.4 Hz, 1H), 6.29-6.07 (m, 2H), 5.33 (dd, J = 5.6, 12.8 Hz, 1H), 4.41- |
| | | 4.27 (m, 4H), 4.00-3.49 (m, 10H), 3.47-3.35 (m, 2H), 3.33 (s, 3H), 3.23 (s, |
| | | 6H), 3.14-3.05 (m, 2H), 3.05-2.75 (m, 4H), 2.74 (s, 4H), 2.35 (s, 2H), 2.32- |
| | | 2.08 (m, 4H), 2.05-1.92 (m, 1H) |
| I-271 | 821.5 | 11.84 (s, 1H), 11.08 (s, 1H), 7.15 (d, J = 8.0 Hz, 1H), 7.08 (s, 1H), 7.01 (d, J = |
| | | 8.0 Hz, 1H), 6.90 (d, J = 7.6 Hz, 1H), 6.89-6.83 (m, 1H), 6.65 (s, 1H), 6.59 |
| | | (d, J = 8.0 Hz, 1H), 6.46 (s, 1H), 5.34 (dd, J = 5.2, 12.8 Hz, 1H), 4.52-4.41 |
| | | (m, 1H), 3.90-3.81 (m, 1H), 3.70 (s, 3H), 3.26-3.20 (m, 6H), 3.19-3.01 (m, |
| | | 7H), 2.98-2.87 (m, 2H), 2.70-2.62 (m, 4H), 2.54 (s, 5H), 2.41-2.34 (m, 3H), |
| | | 2.03 (d, J = 9.2 Hz, 4H), 1.93-1.71 (m, 5H), 1.61-1.42 (m, 1H) |
| I-277 | 783.7 | 11.94 (s, 1H), 10.90 (s, 1H), 7.36 (d, J = 8.0 Hz, 1H), 7.24-7.12 (m, 3H), 6.90- |
| | | 6.80 (m, 1H), 6.65 (s, 1H), 6.58 (d, J = 8.0 Hz, 1H), 6.48 (s, 1H), 6.15-6.06 |
| | | (m, 1H), 4.37-4.26 (m, 2H), 4.17 (dd, J = 5.2, 12.0 Hz, 1H), 3.71 (s, 3H), 3.64- |
| | | 3.56 (m, 2H), 3.23 (s, 5H), 3.18 (d, J = 5.6 Hz, 3H), 3.10-2.94 (m, 3H), 2.82- |
| | | 2.72 (m, 1H), 2.61 (t, J = 7.2 Hz, 2H), 2.55 (s, 4H), 2.39-2.23 (m, 5H), 2.07 |
| | | (d, J = 7.6 Hz, 3H), 2.03-1.94 (m, 1H), 1.84-1.68 (m, 2H) |
| I-282 | 822.6 | 10.89 (s, 1H), 7.77-7.69 (m, 3H), 7.57-7.51 (m, 2H), 7.45-7.37 (m, 2H), |
| | | 7.28-7.21 (m, 2H), 7.09 (br d, J = 8.0 Hz, 2H), 6.20 (br d, J = 9.4 Hz, 1H), |
| | | 4.54-4.45 (m, 1H), 4.36 (q, J = 6.7 Hz, 2H), 4.20-4.13 (m, 1H), 3.94-3.85 |
| | | (m, 1H), 3.25 (br s, 8H), 3.04 (br s, 4H), 2.98-2.85 (m, 3H), 2.82-2.74 (m, |
| | | 3H), 2.63 (br s, 6H), 2.55 (br s, 1H), 2.34-2.23 (m, 1H), 2.02-1.89 (m, 3H), |
| | | 1.82-1.73 (m, 1H), 1.59-1.39 (m, 1H) |
| I-283 | 858.7 | 11.08 (s, 1H), 7.76-7.69 (m, 3H), 7.56-7.49 (m, 2H), 7.41 (dd, J = 1.6, 16.0 |
| | | Hz, 1H), 7.13-7.06 (m, 3H), 7.02 (d, J = 8.0 Hz, 1H), 6.96-6.90 (m, 1H), |
| | | 6.20 (td, J = 2.0, 9.2 Hz, 1H), 5.38-5.30 (m, 1H), 4.53-4.44 (m, 1H), 4.35 (q, |
| | | J = 7.2 Hz, 2H), 3.95-3.85 (m, 1H), 3.33 (br s, 3H), 3.27 (br s, 7H), 3.03 (br |
| | | s, 3H), 2.97-2.80 (m, 7H), 2.76-2.57 (m, 9H), 2.03-1.89 (m, 3H), 1.77 (br |
| | | d, J = 10.8 Hz, 1H), 1.57-1.37 (m, 1H) |
| I-286 | 880.8^{c} (M + 23)⁺ | 11.09 (br s, 1H), 7.74 (d, J = 8.8 Hz, 2H), 7.72-7.68 (m, 1H), 7.54 (d, J = 6.8 |
| | | Hz, 1H), 7.51 (d, J = 1.6 Hz, 1H), 7.41 (dd, J = 1.2, 16.0 Hz, 1H), 7.09 (br d, |
| | | J = 8.4 Hz, 2H), 7.01-6.96 (m, 2H), 6.96-6.92 (m, 1H), 6.20 (td, J = 2.0, 9.6 |
| | | Hz, 1H), 5.38 (br dd, J = 5.6, 12.4 Hz, 1H), 4.53-4.45 (m, 1H), 4.35 (q, J = 7.2 |
| | | Hz, 2H), 3.89 (br d, J = 13.2 Hz, 1H), 3.61 (s, 3H), 3.27 (br s, 6H), 3.18-3.09 |
| | | (m, 3H), 3.07 (br s, 1H), 3.03 (br s, 3H), 2.96-2.85 (m, 4H), 2.77-2.71 (m, |
| | | 1H), 2.70-2.61 (m, 8H), 1.99 (br dd, J = 5.2, 10.4 Hz, 1H), 1.93 (br s, 2H), |
| | | 1.81-1.73 (m, 1H), 1.57-1.37 (m, 1H) |
| I-287 | 858.8 | 11.09 (s, 1H), 7.77-7.69 (m, 3H), 7.54 (d, J = 6.8 Hz, 1H), 7.51 (d, J = 1.6 Hz, |
| | | 1H), 7.41 (dd, J = 1.4, 16.0 Hz, 1H), 7.09 (d, J = 8.4 Hz, 2H), 6.98 (d, J = 5.6 |
| | | Hz, 2H), 6.96-6.92 (m, 1H), 6.20 (td, J = 2.0, 9.2 Hz, 1H), 5.37 (dd, J = 5.2, |
| | | 12.4 Hz, 1H), 4.55-4.43 (m, 1H), 4.35 (q, J = 7.2 Hz, 2H), 3.89 (d, J = 12.8 |
| | | Hz, 1H), 3.61 (s, 3H), 3.27 (s, 6H), 3.19-3.09 (m, 3H), 3.08-3.00 (m, 4H), |
| | | 2.97-2.85 (m, 4H), 2.79-2.71 (m, 1H), 2.70-2.60 (m, 8H), 2.03-1.97 (m, |
| | | 1H), 1.93 (s, 2H), 1.77 (d, J = 10.8 Hz, 1H), 1.58-1.40 (m, 1H) |
| I-290 | 825.8 | 11.96-11.57 (m, 1H), 11.11 (s, 1H), 7.33-6.99 (m, 2H), 6.99-6.82 (m, 2H), |
| | | 6.79-6.56 (m, 2H), 6.46 (s, 1H), 5.39 (d, J = 8.4 Hz, 1H), 4.60-4.34 (m, 1H), |
| | | 3.86 (d, J = 13.6 Hz, 1H), 3.72 (s, 3H), 3.68-3.58 (m, 3H), 3.28-3.23 (m, |
| | | 4H), 3.22-3.10 (m, 6H), 3.08-2.97 (m, 3H), 2.97-2.84 (m, 2H), 2.78-2.70 |
| | | (m, 2H), 2.69-2.64 (m, 3H), 2.64-2.52 (m, 4H), 2.10-2.01 (m, 3H), 2.01- |
| | | 1.90 (m, 2H), 1.87-1.69 (m, 2H), 1.66-1.36 (m, 1H) |
| I-294 | 797.6 | 11.74 (s, 1H), 10.89 (s, 1H), 7.39 (s, 1H), 7.30-7.20 (m, 2H), 7.15 (d, J = 8.4 |
| | | Hz, 1H), 6.87-6.89 (m, 1H), 6.68 (d, J = 13.6 Hz, 2H), 6.63-6.54 (m, 1H), |
| | | 4.60-4.32 (m, 1H), 4.17-4.19 (m, 1H), 3.86 (d, J = 12.4 Hz, 1H), 3.70 (s, |
| | | 3H), 3.23 (s, 4H), 3.13-3.05 (m, 2H), 3.02 (s, 3H), 2.97-2.91 (m, 1H), 2.90- |
| | | 2.69 (m, 4H), 2.66-2.60 (m, 3H), 2.60-2.51 (m, 4H), 2.34-2.24 (m, 1H), |
| | | 2.03 (d, J = 8.8 Hz, 3H), 2.00-1.90 (m, 2H), 1.88-1.69 (m, 2H), 1.66-1.37 |
| | | (m, 1H), 0.72 (d, J = 6.4 Hz, 2H), 0.61 (s, 2H) |
| I-297 | 807.5 | 11.84 (s, 1H), 11.10 (s, 1H), 7.16 (d, J = 8.0 Hz, 1H), 7.13-6.96 (m, 2H), 6.96- |
| | | 6.91 (m, 1H), 6.88 (dd, J = 6.0, 10.0 Hz, 1H), 6.67 (s, 1H), 6.60 (d, J = 7.6 |
| | | Hz, 1H), 6.47 (s, 1H), 5.38 (dd, J = 4.8, 12.4 Hz, 1H), 4.59-4.31 (m, 1H), 3.93- |
| | | 3.79 (m, 1H), 3.71 (s, 3H), 3.62 (s, 3H), 3.28-3.20 (m, 6H), 3.12 (dd, J = 8.4, |
| | | 15.6 Hz, 6H), 3.05-2.94 (m, 2H), 2.91-2.84 (m, 1H), 2.80-2.69 (m, 2H), |
| | | 2.68 (s, 3H), 2.66-2.63 (m, 2H), 2.62-2.54 (m, 2H), 2.03 (d, J = 8.8 Hz, 4H), |
| | | 1.95-1.80 (m, 2H), 1.66 (s, 2H) |
| I-300 | 861.5 | 12.11-11.95 (m, 1H), 11.18-10.95 (m, 1H), 7.42 (s, 1H), 7.11 (s, 2H), 7.01 |
| | | (s, 1H), 6.98-6.91 (m, 3H), 6.55-6.49 (m, 1H), 5.39-5.29 (m, 1H), 4.53- |
| | | 4.40 (m, 1H), 3.93-3.79 (m, 1H), 3.33 (s, 3H), 3.27 (s, 4H), 2.99 (s, 8H), 2.92- |
| | | 2.81 (m, 3H), 2.77-2.67 (m, 2H), 2.63 (s, 7H), 2.03 (d, J = 9.6 Hz, 4H), 1.95- |
| | | 1.89 (m, 1H), 1.87-1.73 (m, 2H), 1.62-1.42 (m, 1H) |
| I-307 | 858.6 | 11.09 (s, 1H), 7.80 (br d, J = 8.4 Hz, 2H), 7.74-7.69 (m, 1H), 7.59-7.52 (m, |
| | | 2H), 7.42 (br d, J = 16.5 Hz, 1H), 7.20-7.14 (m, 3H), 7.11 (d, J = 8.0 Hz, 1H), |
| | | 6.98 (d, J = 8.1 Hz, 1H), 6.20 (br d, J = 9.4 Hz, 1H), 5.37 (dd, J = 5.3, 12.8 Hz, |
| | | 1H), 4.53-4.45 (m, 1H), 4.36 (q, J = 6.7 Hz, 2H), 4.00-3.87 (m, 3H), 3.68 (br |
| | | d, J = 5.9 Hz, 3H), 3.45 (br s, 2H), 3.35 (s, 3H), 3.29-3.19 (m, 7H), 3.17-3.12 |
| | | (m, 2H), 3.04 (br s, 3H), 2.97-2.86 (m, 4H), 2.74-2.60 (m, 3H), 2.07-1.91 |
| | | (m, 3H), 1.77 (br d, J = 11.4 Hz, 1H), 1.59-1.38 (m, 1H) |
| I-315 | 807.6 | 11.84 (s, 1H), 11.09 (s, 1H), 7.15 (d, J = 8.4 Hz, 1H), 7.11 (s, 1H), 7.02 (d, J = |
| | | 8.0 Hz, 1H), 6.93 (d, J = 8.4 Hz, 1H), 6.88 (dd, J = 6.0, 10.0 Hz, 1H), 6.66 (s, |
| | | 1H), 6.60 (d, J = 8.0 Hz, 1H), 6.46 (s, 1H), 5.34 (dd, J = 5.2, 12.8 Hz, 1H), 4.52- |
| | | 4.39 (m, 1H), 3.85 (d, J = 12.4 Hz, 1H), 3.71 (s, 3H), 3.32-3.19 (m, 10H), |
| | | 3.15-3.02 (m, 5H), 2.86-2.80 (m, 2H), 2.76-2.53 (m, 10H), 2.03 (d, J = 8.8 |
| | | Hz, 4H), 1.94-1.72 (m, 3H), 1.63-1.37 (m, 1H) |
| I-317 | 797.4 | 11.84 (s, 1H), 10.89 (s, 1H), 7.39 (s, 1H), 7.28-7.21 (m, 2H), 7.16 (br d, J = |
| | | 8.3 Hz, 1H), 6.95-6.85 (m, 1H), 6.67 (s, 1H), 6.61 (br d, J = 8.1 Hz, 1H), 6.47 |
| | | (br s, 1H), 4.52-4.40 (m, 1H), 4.37-4.25 (m, 1H), 4.17 (dd, J = 5.1, 12.3 Hz, |
| | | 1H), 3.72 (s, 3H), 3.30 (s, 1H), 3.25 (br s, 3H), 3.20-3.03 (m, 6H), 2.84-2.77 |
| | | (m, 3H), 2.75-2.71 (m, 1H), 2.63 (br d, J = 5.8 Hz, 6H), 2.55 (br d, J = 3.8 Hz, |
| | | 2H), 2.32-2.25 (m, 1H), 1.98 (ddd, J = 4.8, 8.0, 13.0 Hz, 4H), 1.90-1.70 (m, |
| | | 2H), 1.63-1.43 (m, 1H), 0.81-0.64 (m, 4H) |
| I-324 | 843.5 | 11.53-11.44 (m, 1H), 11.09 (s, 1H), 7.76-7.71 (m, 1H), 7.46-7.40 (m, 1H), |
| | | 7.23 (s, 1H), 7.08 (s, 1H), 7.04-6.99 (m, 2H), 6.94-6.89 (m, 2H), 6.26-6.19 |
| | | (m, 2H), 5.35 (dd, J = 5.6, 12.8 Hz, 1H), 4.41-4.29 (m, 4H), 3.63-3.53 (m, |
| | | 2H), 3.30-3.07 (m, 6H), 3.05-2.96 (m, 3H), 2.95-2.87 (m, 2H), 2.81-2.72 |
| | | (m, 3H), 2.66 (br dd, J = 4.0, 15.6 Hz, 2H), 2.58 (br s, 4H), 2.55-2.53 (m, 2H), |
| | | 2.46-2.38 (m, 4H), 2.35-2.29 (m, 2H), 2.24 (br d, J = 3.6 Hz, 1H), 2.05-1.89 |
| | | (m, 6H), 1.70-1.60 (m, 2H), 1.54-1.45 (m, 2H) |
| I-330 | 843.4 | 11.55-11.44 (m, 1H), 11.09 (s, 1H), 7.72 (s, 1H), 7.45-7.39 (m, 1H), 7.24 (s, |
| | | 1H), 7.09 (s, 1H), 7.05-6.96 (m, 3H), 6.92 (br d, J = 8.0 Hz, 1H), 6.20 (br s, |
| | | 2H), 5.35 (dd, J = 5.2, 12.8 Hz, 1H), 4.41-4.25 (m, 4H), 3.65-3.52 (m, 2H), |
| | | 3.32-3.10 (m, 6H), 3.10-2.84 (m, 6H), 2.83-2.63 (m, 5H), 2.62-2.51 (m, |
| | | 8H), 2.36-2.19 (m, 4H), 2.18-1.95 (m, 6H), 1.56 (br s, 4H) |
| I-337 | 859.6 | 12.09 (s, 1H), 11.11 (s, 1H), 7.48-7.27 (m, 1H), 7.21-7.09 (m, 1H), 7.07- |
| | | 6.94 (m, 2H), 6.93-6.85 (m, 2H), 6.84-6.75 (m, 1H), 6.54 (s, 1H), 6.20-6.04 |
| | | (m, 1H), 5.45-5.27 (m, 1H), 4.42-4.25 (m, 2H), 3.63 (s, 3H), 3.61-3.54 (m, |
| | | 2H), 3.49-3.35 (m, 2H), 3.27 (s, 4H), 3.20-3.03 (m, 6H), 3.01-2.80 (m, 2H), |
| | | 2.78-2.68 (m, 2H), 2.66 (d, J = 6.4 Hz, 3H), 2.61 (s, 2H), 2.42-2.34 (m, 1H), |
| | | 2.33-2.23 (m, 1H), 2.07 (d, J = 9.2 Hz, 3H), 2.04-1.94 (m, 1H) |
| I-338 | 885.3 | 12.11-11.86 (m, 1H), 11.22-11.04 (m, 1H), 7.40-7.26 (m, 1H), 7.15-7.08 |
| | | (m, 1H), 7.02 (dd, J = 4.4, 8.4 Hz, 1H), 6.96-6.90 (m, 2H), 6.88 (d, J = 6.4 |
| | | Hz, 1H), 6.84-6.69 (m, 2H), 6.19-6.07 (m, 1H), 5.33 (s, 1H), 4.60-4.16 (m, |
| | | 2H), 3.68-3.56 (m, 5H), 3.28 (br d, J = 1.2 Hz, 6H), 3.17-3.01 (m, 6H), 2.81- |
| | | 2.57 (m, 10H), 2.42-2.22 (m, 4H), 2.08 (d, J = 8.4 Hz, 3H), 2.04-1.97 (m, |
| | | 1H), 0.83-0.55 (m, 4H) |
| I-339 | 843.6 | 11.56-11.45 (m, 1H), 11.11 (br s, 1H), 8.31 (s, 1H), 7.72 (d, J = 1.6 Hz, 1H), |
| | | 7.47-7.35 (m, 1H), 7.24 (s, 1H), 6.99 (br s, 3H), 6.94 (br s, 1H), 6.26-6.17 |
| | | (m, 2H), 5.38 (br dd, J = 5.2, 12.4 Hz, 1H), 4.40-4.28 (m, 4H), 3.70-3.47 (m, |
| | | 10H), 3.13-3.07 (m, 3H), 2.99 (td, J = 6.8, 12.8 Hz, 4H), 2.89 (br d, J = 12.0 |
| | | Hz, 1H), 2.78-2.66 (m, 2H), 2.66-2.55 (m, 7H), 2.35-2.20 (m, 4H), 2.17- |
| | | 1.96 (m, 6H), 1.57 (br s, 4H) |
| I-341 | 843.6 | 11.56-11.42 (m, 1H), 11.10 (s, 1H), 7.74 (d, J = 2.0 Hz, 1H), 7.45-7.40 (m, |
| | | 1H), 7.23 (s, 1H), 7.02-6.96 (m, 3H), 6.95-6.89 (m, 2H), 6.27-6.19 (m, 2H), |
| | | 5.37 (br dd, J = 5.6, 12.0 Hz, 1H), 4.41-4.29 (m, 4H), 3.64-3.58 (m, 4H), |
| | | 3.55 (br s, 1H), 3.31-3.18 (m, 6H), 3.10-3.04 (m, 3H), 3.03-2.89 (m, 5H), |
| | | 2.77-2.64 (m, 3H), 2.62-2.56 (m, 6H), 2.44 (br d, J = 3.2 Hz, 1H), 2.32 (br |
| | | d, J = 8.4 Hz, 2H), 2.24 (br d, J = 3.2 Hz, 1H), 2.03-1.92 (m, 5H), 1.71-1.60 |
| | | (m, 2H), 1.54-1.45 (m, 2H) |
| I-342 | 841.4 | 12.07 (br s, 1H), 11.09 (s, 1H), 7.39-7.10 (m, 2H), 7.01-6.92 (m, 4H), 6.92- |
| | | 6.86 (m, 1H), 6.81 (s, 1H), 6.54 (br d, J = 4.4 Hz, 1H), 6.19-6.06 (m, 1H), |
| | | 5.37 (dd, J = 5.6, 12.8 Hz, 1H), 4.37-4.28 (m, 2H), 3.65-3.57 (m, 5H), 3.28 |
| | | (br d, J = 4.4 Hz, 6H), 3.20-3.07 (m, 6H), 2.93-2.84 (m, 1H), 2.71-2.64 (m, |
| | | 7H), 2.33 (s, 2H), 2.30-2.22 (m, 1H), 2.07 (d, J = 9.2 Hz, 3H), 2.03-1.96 (m, |
| | | 1H) |
| I-347 | 873.7 | 12.08 (s, 1H), 11.10 (s, 1H), 8.15 (s, 1H), 7.34-7.36 (m, 1H), 7.21-6.96 (m, |
| | | 2H), 6.95-6.84 (m, 3H), 6.79 (s, 1H), 6.54 (d, J = 2.4 Hz, 1H), 6.20-6.06 (m, |
| | | 1H), 5.44-5.27 (m, 1H), 4.44-4.24 (m, 2H), 3.65-3.62 (m, 1H), 3.60 (s, 3H), |
| | | 3.57 (d, J = 2.8 Hz, 1H), 3.21 (s, 4H), 3.14-2.94 (m, 6H), 2.94-2.80 (m, 2H), |
| | | 2.57 (s, 4H), 2.54 (s, 3H), 2.46 (d, J = 13.2 Hz, 2H), 2.42-2.25 (m, 2H), 2.07 |
| | | (d, J = 8.8 Hz, 3H), 2.01-1.93 (m, 1H), 1.85-1.75 (m, 2H) |
| I-349 | 771.8 | 11.84 (s, 1H), 10.90 (s, 1H), 7.37 (d, J = 8.0 Hz, 1H), 7.28-7.10 (m, 3H), 6.88 |
| | | (dd, J = 6.0, 10.0 Hz, 1H), 6.69-6.53 (m, 2H), 6.46 (s, 1H), 4.53-4.38 (m, |
| | | 1H), 4.17 (dd, J = 4.8, 12.1 Hz, 1H), 3.91-3.79 (m, 1H), 3.71 (s, 3H), 3.29- |
| | | 3.26 (m, 5H), 3.23 (s, 5H), 3.14-3.03 (m, 4H), 2.98-2.90 (m, 1H), 2.83-2.72 |
| | | (m, 3H), 2.62-2.54 (m, 6H), 2.35-2.24 (m, 1H), 2.03 (d, J = 9.2 Hz, 3H), 1.93 |
| | | (d, J = 8.4 Hz, 1H), 1.87-1.70 (m, 2H), 1.63-1.38 (m, 1H) |
| I-350 | 877.7 | 12.17 (s, 1H), 11.12 (s, 1H), 7.61-7.40 (m, 1H), 7.16 (d, J = 8.4 Hz, 1H), 7.13- |
| | | 7.05 (m, 2H), 7.02-6.81 (m, 2H), 6.52 (s, 1H), 6.12 (s, 1H), 5.41 (dd, J = 5.2, |
| | | 12.4 Hz, 1H), 4.32 (d, J = 4.8 Hz, 2H), 4.05 (d, J = 10.0 Hz, 2H), 3.80 (s, 2H), |
| | | 3.68 (s, 3H), 3.61 (d, J = 6.0 Hz, 2H), 3.53-3.41 (m, 4H), 3.38-3.24 (m, 4H), |
| | | 3.24-2.95 (m, 6H), 2.94-2.81 (m, 1H), 2.78-2.58 (m, 2H), 2.38 (d, J = 2.4 |
| | | Hz, 1H), 2.27 (d, J = 1.2 Hz, 1H), 2.13-2.03 (m, 3H), 2.03-1.94 (m, 1H) |
| I-351 | 823.3 | 10.91-10.87 (m, 1H), 8.14-8.07 (m, 1H), 7.74 (d, J = 8.8 Hz, 2H), 7.70 (d, |
| | | J = 9.2 Hz, 1H), 7.58-7.54 (m, 1H), 7.53-7.51 (m, 1H), 7.39 (s, 1H), 7.27- |
| | | 7.21 (m, 2H), 7.12-7.06 (m, 2H), 4.65-4.58 (m, 2H), 4.53-4.44 (m, 1H), |
| | | 4.17 (dd, J = 5.2, 12.4 Hz, 1H), 3.97-3.87 (m, 1H), 3.31-3.20 (m, 8H), 3.10- |
| | | 3.00 (m, 6H), 2.94-2.83 (m, 1H), 2.83-2.78 (m, 2H), 2.77-2.72 (m, 1H), |
| | | 2.62 (d, J = 4.4 Hz, 6H), 2.57-2.53 (m, 1H), 2.34-2.27 (m, 1H), 2.00-1.91 |
| | | (m, 3H), 1.81-1.75 (m, 1H), 1.59-1.42 (m, 1H). |
| I-352 | 823.4 | 10.90 (s, 1H), 8.15-8.07 (m, 1H), 7.74 (d, J = 8.8 Hz, 2H), 7.70 (d, J = 9.2 Hz, |
| | | 1H), 7.58-7.54 (m, 1H), 7.52 (d, J = 2.0 Hz, 1H), 7.39 (s, 1H), 7.27-7.22 (m, |
| | | 2H), 7.12-7.07 (m, 2H), 4.62 (q, J = 7.2 Hz, 2H), 4.53-4.45 (m, 1H), 4.17 |
| | | (dd, J = 5.2, 12.4 Hz, 1H), 3.96-3.87 (m, 1H), 3.30-3.21 (m, 8H), 3.09-3.01 |
| | | (m, 6H), 2.92-2.83 (m, 1H), 2.83-2.78 (m, 2H), 2.78-2.72 (m, 1H), 2.63 (s, |
| | | 6H), 2.55 (br d, J = 2.4 Hz, 1H), 2.28 (dd, J = 4.4, 12.8 Hz, 1H), 2.00-1.92 (m, |
| | | 3H), 1.82-1.76 (m, 1H), 1.59-1.43 (m, 1H). |
| I-354 | 916.8 | 12.14 (s, 1H), 11.10 (s, 1H), 7.43 (d, J = 8.0 Hz, 1H), 7.05 (d, J = 8.8 Hz, 1H), |
| | | 7.02-6.97 (m, 1H), 6.97-6.94 (m, 1H), 6.91 (s, 1H), 6.89-6.82 (m, 1H), 6.53 |
| | | (s, 1H), 6.12 (s, 1H), 5.37 (dd, J = 5.2, 12.4 Hz, 1H), 4.56 (s, 1H), 4.36 (s, 1H), |
| | | 3.85 (s, 1H), 3.65 (s, 1H), 3.60 (s, 3H), 3.22 (s, 4H), 3.20-3.00 (m, 6H), 2.99- |
| | | 2.93 (m, 2H), 2.90-2.82 (m, 1H), 2.74-2.58 (m, 3H), 2.53 (s, 4H), 2.46 (s, |
| | | 1H), 2.40 (s, 1H), 2.26 (s, 1H), 2.13-1.93 (m, 2H), 1.80 (d, J = 6.4 Hz, 2H), |
| | | 1.06-0.59 (m, 4H) |
| I-355 | 860.7 | 11.54-11.44 (m, 1H), 11.09 (s, 1H), 8.19 (s, 1H), 7.69-7.63 (m, 1H), 7.56- |
| | | 7.52 (m, 1H), 7.27-7.22 (m, 1H), 7.09 (s, 1H), 7.02-7.00 (m, 1H), 6.98 (s, |
| | | 2H), 6.91 (d, J = 8.0 Hz, 1H), 6.22 (s, 1H), 5.34 (dd, J = 5.6, 12.6 Hz, 1H), 4.37 |
| | | (s, 2H), 3.65-3.58 (m, 2H), 3.37-3.34 (m, 3H), 3.25 (t, J = 7.2 Hz, 5H), 3.21- |
| | | 3.11 (m, 3H), 3.06-2.96 (m, 2H), 2.96-2.87 (m, 3H), 2.85-2.71 (m, 3H), |
| | | 2.71-2.62 (m, 2H), 2.62-2.51 (m, 6H), 2.34 (d, J = 2.4 Hz, 2H), 2.22 (s, 2H), |
| | | 2.18-1.89 (m, 6H), 1.55 (s, 4H) |
| I-357 | 902.5 | 11.11 (s, 1H), 7.71 (br d, J = 10.0 Hz, 3H), 7.59-7.44 (m, 2H), 7.44-7.35 (m, |
| | | 1H), 7.14-7.06 (m, 2H), 7.00 (br d, J = 3.6 Hz, 1H), 6.94-6.86 (m, 1H), 6.22- |
| | | 6.13 (m, 2H), 5.38 (br dd, J = 4.8, 12.4 Hz, 1H), 4.39-4.34 (m, 3H), 4.29 (br |
| | | s, 1H), 3.73-3.53 (m, 6H), 3.52-3.37 (m, 3H), 3.26 (br s, 3H), 3.12 (br s, 2H), |
| | | 3.04-2.91 (m, 4H), 2.90-2.81 (m, 1H), 2.76-2.59 (m, 8H), 2.35-2.25 (m, |
| | | 2H), 2.03-1.97 (m, 1H), 1.71-1.59 (m, 2H), 0.77 (br s, 3H) |
| I-358 | 884.5 | 11.20-10.99 (m, 1H), 7.73 (br s, 3H), 7.43 (s, 3H), 7.09 (br d, J = 8.0 Hz, 2H), |
| | | 6.98 (br d, J = 4.8 Hz, 3H), 6.24-6.11 (m, 2H), 5.43-5.30 (m, 1H), 4.35 (br |
| | | s, 4H), 3.61 (s, 5H), 3.54-3.42 (m, 2H), 3.26 (br s, 6H), 3.17-3.10 (m, 2H), |
| | | 3.01-2.85 (m, 4H), 2.67 (br s, 8H), 2.35-2.23 (m, 2H), 2.05-1.96 (m, 1H), |
| | | 1.70-1.56 (m, 2H), 0.94-0.69 (m, 3H) |
| I-360 | 839.4 | 11.85 (s, 1H), 11.11 (s, 1H), 7.15 (d, J = 8.4 Hz, 1H), 6.99-7.03 (m, 1H), 6.94- |
| | | 6.83 (m, 2H), 6.65 (s, 1H), 6.59 (d, J = 8.8 Hz, 1H), 6.46-6.49 (m, 1H), 5.37- |
| | | 5.4 (m, 1H), 4.55-4.38 (m, 1H), 3.92-3.80 (m, 1H), 3.71 (s, 3H), 3.65-3.55 |
| | | (m, 3H), 3.24 (s, 4H), 3.19-3.07 (m, 6H), 3.07-2.96 (m, 4H), 2.91-2.81 (m, |
| | | 1H), 2.76-2.63 (m, 2H), 2.63-2.56 (m, 2H), 2.54 (s, 3H), 2.49-2.44 (m, 2H), |
| | | 2.03 (d, J = 8.8 Hz, 3H), 1.99-1.90 (m, 2H), 1.89-1.71 (m, 4H), 1.67-1.34 |
| | | (m, 1H) |
| I-363 | 899.7 | 12.07 (s, 1H), 11.10 (s, 1H), 7.33 (d, J = 8.8 Hz, 1H), 7.23-7.06 (m, 1H), 7.02- |
| | | 6.91 (m, 2H), 6.91-6.84 (m, 2H), 6.79 (s, 1H), 6.54 (s, 1H), 6.14 (d, J = 12.0 |
| | | Hz, 1H), 5.37-5.39 (m, 1H), 4.56 (s, 1H), 4.44-4.28 (m, 1H), 3.85-3.86 (m, |
| | | 1H), 3.71-3.63 (m, 1H), 3.60 (s, 3H), 3.28-3.20 (m, 4H), 3.19-2.99 (m, 6H), |
| | | 2.98-2.89 (m, 2H), 2.89-2.80 (m, 1H), 2.76-2.62 (m, 2H), 2.61-2.55 (m, |
| | | 2H), 2.54 (s, 3H), 2.47-2.45 (m, 1H), 2.42-2.22 (m, 2H), 2.14-1.89 (m, 2H), |
| | | 1.86-1.73 (m, 2H), 0.83-0.65 (m, 4H) |
| I-364 | 444.3^{b} | 12.21-11.92 (m, 1H), 11.30-10.96 (m, 1H), 7.39-7.33 (m, 1H), 7.33-7.11 |
| | | (m, 1H), 7.02 (dd, J = 4.4, 8.6 Hz, 1H), 6.98-6.93 (m, 1H), 6.91 (br d, J = 1.4 |
| | | Hz, 1H), 6.90-6.87 (m, 1H), 6.82 (s, 1H), 6.59-6.44 (m, 1H), 6.19-6.10 (m, |
| | | 1H), 5.39 (dd, J = 5.4, 12.5 Hz, 1H), 4.40-4.28 (m, 2H), 3.66-3.57 (m, 5H), |
| | | 3.47 (br s, 2H), 3.28 (br s, 4H), 3.21-3.07 (m, 4H), 2.95-2.84 (m, 1H), 2.80- |
| | | 2.71 (m, 1H), 2.71-2.58 (m, 8H), 2.38 (br d, J = 2.6 Hz, 1H), 2.27 (br d, J = |
| | | 1.0 Hz, 1H), 2.08 (d, J = 8.6 Hz, 3H), 2.05-1.97 (m, 1H), 1.65-1.53 (m, 2H), |
| | | 0.96-0.71 (m, 3H) |
| I-365 | 837.7 | 12.13 (br s, 1H), 11.10 (br s, 1H), 7.33 (d, J = 8.5 Hz, 1H), 7.11 (d, J = 1.8 Hz, |
| | | 1H), 7.01 (br s, 1H), 6.99 (br d, J = 5.4 Hz, 1H), 6.97-6.93 (m, 1H), 6.88 (d, |
| | | J = 6.0 Hz, 1H), 6.51-6.32 (m, 1H), 6.21-6.08 (m, 1H), 5.38 (br dd, J = 5.4, |
| | | 12.5 Hz, 1H), 4.39-4.29 (m, 2H), 3.66-3.58 (m, 5H), 3.53-3.41 (m, 3H), |
| | | 3.28 (br s, 4H), 3.19-3.07 (m, 4H), 2.96-2.85 (m, 1H), 2.80-2.72 (m, 1H), |
| | | 2.71-2.59 (m, 8H), 2.38 (br s, 1H), 2.27 (br s, 1H), 2.08 (d, J = 6.3 Hz, 3H), |
| | | 2.05-1.96 (m, 1H), 1.59 (qd, J = 7.4, 14.6 Hz, 2H), 1.00-0.71 (m, 3H) |
| I-366 | 803.4 | 12.24-11.97 (m, 1H), 7.56-7.50 (m, 2H), 7.08 (br d, J = 8.4 Hz, 2H), 7.01- |
| | | 6.94 (m, 4H), 6.87-6.79 (m, 1H), 6.19-6.13 (m, 1H), 5.42-5.34 (m, 1H), |
| | | 4.38-4.33 (m, 2H), 3.64-3.59 (m, 5H), 3.52-3.46 (m, 2H), 3.25 (br s, 6H), |
| | | 3.17-3.11 (m, 3H), 2.94-2.85 (m, 1H), 2.78-2.72 (m, 1H), 2.70-2.64 (m, |
| | | 7H), 2.41-2.36 (m, 2H), 2.30-2.26 (m, 1H), 2.09 (d, J = 7.2 Hz, 3H), 2.03- |
| | | 1.98 (m, 1H), 1.65-1.58 (m, 2H), 0.94-0.80 (m, 3H) |
| I-367 | 869.5 | 12.08 (br s, 1H), 11.15-11.06 (m, 1H), 7.39-7.33 (m, 1H), 7.32-7.10 (m, |
| | | 1H), 6.99 (br d, J = 5.5 Hz, 1H), 6.95 (br d, J = 6.3 Hz, 1H), 6.92-6.87 (m, |
| | | 1H), 6.83 (s, 1H), 6.53 (br s, 1H), 6.20-6.09 (m, 1H), 5.38 (dd, J = 5.4, 12.6 |
| | | Hz, 1H), 4.39-4.30 (m, 2H), 3.66-3.57 (m, 5H), 3.47 (br s, 3H), 3.33 (br s, |
| | | 6H), 3.19-3.07 (m, 4H), 2.96-2.85 (m, 1H), 2.80-2.72 (m, 1H), 2.72-2.63 |
| | | (m, 7H), 2.38 (br d, J = 3.4 Hz, 1H), 2.28 (br s, 1H), 2.08 (d, J = 8.5 Hz, 3H), |
| | | 2.04-1.97 (m, 1H), 1.59 (sxt, J = 7.3 Hz, 2H), 1.01-0.71 (m, 3H) |
| I-368 | 968.4 | 11.11 (s, 1H), 7.72 (dd, J = 2.0, 12.0 Hz, 1H), 7.59-7.48 (m, 1H), 7.47-7.36 |
| | | (m, 2H), 7.35-7.28 (m, 1H), 7.20-7.08 (m, 1H), 7.02 (dd, J = 4.4, 8.8 Hz, |
| | | 1H), 6.99-6.88 (m, 2H), 6.87-6.78 (m, 1H), 6.21 (t, J = 2.0 Hz, 1H), 6.19- |
| | | 6.09 (m, 1H), 5.39 (dd, J = 5.6, 12.4 Hz, 1H), 4.41-4.32 (m, 3H), 4.28 (br s, |
| | | 1H), 3.68-3.61 (m, 4H), 3.60-3.56 (m, 1H), 3.42 (br s, 2H), 3.20 (br s, 1H), |
| | | 3.17-3.10 (m, 2H), 3.02-2.92 (m, 4H), 2.71-2.59 (m, 8H), 2.37-2.22 (m, |
| | | 2H), 2.05-1.96 (m, 1H), 1.60-1.49 (m, 2H), 0.96-0.55 (m, 3H) |
| I-370 | 874.3 | 11.55-10.62 (m, 1H), 7.77-7.73 (m, 2H), 7.56-7.53 (m, 1H), 7.46-7.37 (m, |
| | | 1H), 7.14-7.06 (m, 2H), 7.06-6.96 (m, 1H), 6.95-6.86 (m, 1H), 6.31-6.06 |
| | | (m, 2H), 5.44-5.30 (m, 1H), 4.39-4.33 (m, 3H), 4.30 (s, 1H), 3.68-3.58 (m, |
| | | 5H), 3.30-3.26 (m, 6H), 3.26-3.25 (m, 1H), 3.21-3.08 (m, 3H), 3.08-3.01 |
| | | (m, 3H), 3.00-2.91 (m, 2H), 2.91-2.83 (m, 1H), 2.76-2.59 (m, 8H), 2.43- |
| | | 2.21 (m, 3H), 2.05-1.97 (m, 1H) |
| I-373 | 871.3 | 10.85 (s, 1H), 8.10 (d, J = 12.0 Hz, 1H), 7.70 (s, 1H), 7.53 (s, 1H), 7.46-7.40 |
| | | (m, 1H), 7.38-7.29 (m, 2H), 7.16-7.03 (m, 2H), 7.02-6.94 (m, 2H), 6.88- |
| | | 6.79 (m, 1H), 6.18-6.10 (m, 1H), 4.62 (q, J = 6.8 Hz, 2H), 4.37-4.28 (m, 2H), |
| | | 3.87 (br dd, J = 4.8, 12.0 Hz, 1H), 3.67-3.58 (m, 2H), 3.29-3.18 (m, 7H), |
| | | 3.14-2.97 (m, 6H), 2.81 (br s, 2H), 2.71-2.56 (m, 7H), 2.35-2.16 (m, 3H), |
| | | 2.04 (br d, J = 4.4 Hz, 1H) |
| I-374 | 805.3 | 10.87-10.81 (m, 1H), 8.14-8.04 (m, 1H), 7.77-7.66 (m, 3H), 7.54 (s, 2H), |
| | | 7.35-7.28 (m, 1H), 7.10-7.03 (m, 3H), 7.02-6.98 (m, 1H), 6.20-6.13 (m, |
| | | 1H), 4.62 (d, J = 7.2 Hz, 2H), 4.38-4.30 (m, 2H), 3.87 (dd, J = 4.8, 12.0 Hz, |
| | | 1H), 3.68-3.59 (m, 2H), 3.29-3.22 (m, 7H), 3.13-3.01 (m, 5H), 2.84-2.78 |
| | | (m, 2H), 2.72-2.55 (m, 8H), 2.36 (br s, 1H), 2.30-2.17 (m, 2H), 2.02 (qd, |
| | | J = 4.4, 13.2 Hz, 1H) |
| I-375 | 952.5 | 11.2 (br s, 1H), 7.71 (br d, J = 11.6 Hz, 1H), 7.59 (s, 1H), 7.48-7.28 (m, 3H), |
| | | 7.14-7.10 (m, 1H), 6.97-6.88 (m, 2H), 6.80 (br s, 1H), 6.27-6.04 (m, 2H), |
| | | 5.40 (br dd, J = 4.8, 12.5 Hz, 1H), 4.44-4.24 (m, 4H), 3.84-3.69 (m, 5H), |
| | | 3.67-3.54 (m, 2H), 3.26 (br s, 5H), 3.12 (br s, 1H), 3.04-2.83 (m, 6H), 2.77- |
| | | 2.59 (m, 6H), 2.37-2.23 (m, 2H), 2.09-1.99 (m, 1H), 0.81-0.53 (m, 4H) |
| I-376 | 967.5 | 11.25-10.93 (m, 1H), 8.11 (d, J = 11.6 Hz, 1H), 7.73-7.56 (m, 2H), 7.44- |
| | | 7.31 (m, 2H), 7.17-6.87 (m, 4H), 6.82 (br s, 1H), 6.15 (br d, J = 12.8 Hz, 1H), |
| | | 5.39 (dd, J = 5.2, 12.4 Hz, 1H), 4.63 (q, J = 7.2 Hz, 2H), 4.34 (br d, J = 15.6 |
| | | Hz, 2H), 3.66-3.59 (m, 5H), 3.30 (br s, 4H), 3.16-3.07 (m, 5H), 3.01 (s, 3H), |
| | | 2.93-2.83 (m, 1H), 2.72-2.61 (m, 8H), 2.38-2.27 (m, 2H), 2.06-1.96 (m, |
| | | 1H), 0.75 (br d, J = 6.8 Hz, 2H), 0.61 (br s, 2H) |
| I-377 | 966.5 | 11.11 (s, 1H), 7.72 (dd, J = 2.0, 11.5 Hz, 1H), 7.60 (s, 1H), 7.44-7.37 (m, 2H), |
| | | 7.35-7.29 (m, 1H), 7.16-6.95 (m, 3H), 6.94-6.88 (m, 1H), 6.82 (s, 1H), 6.24- |
| | | 6.10 (m, 2H), 5.39 (br dd, J = 5.2, 12.4 Hz, 1H), 4.40-4.34 (m, 3H), 4.29 (br |
| | | d, J = 1.2 Hz, 1H), 3.67-3.58 (m, 5H), 3.31-3.27 (m, 5H), 3.13 (br d, J = 6.4 |
| | | Hz, 3H), 3.01 (s, 3H), 2.99-2.94 (m, 2H), 2.91-2.84 (m, 1H), 2.77-2.71 (m, |
| | | 1H), 2.71-2.64 (m, 6H), 2.61 (br s, 1H), 2.30-2.24 (m, 1H), 2.06-1.97 (m, |
| | | 1H), 0.75 (br d, J = 6.6 Hz, 2H), 0.61 (br s, 2H) |
| I-381 | 850.7 | 12.72-12.22 (m, 1H), 11.08 (s, 1H), 10.89-10.62 (m, 1H), 7.72 (dd, J = 2.0, |
| | | 9.2 Hz, 1H), 7.68-7.46 (m, 1H), 7.43 (dd, J = 1.2, 13.2 Hz, 1H), 7.40- 7.25 |
| | | (m, 1H), 7.02 (d, J = 8.4 Hz, 1H), 6.95 (d, J = 1.6 Hz, 1H), 6.80-6.60 (m, 1H), |
| | | 6.21 (td, J = 2.0, 9.6 Hz, 1H), 5.33 (dd, J = 5.2, 12.8 Hz, 1H), 4.49 (d, J = 8.8 |
| | | Hz, 1H), 4.41-4.27 (m, 2H), 3.92-3.87 (m, 2H), 3.83-3.59 (m, 8H), 3.38- |
| | | 3.34 (m, 1H), 3.33 (s, 3H), 3.29 (s, 1H), 3.25-3.14 (m, 6H), 3.13-2.99 (m, |
| | | 4H), 2.98-2.79 (m, 4H), 2.76-2.51 (m, 4H), 2.30-2.02 (m, 4H), 1.99 (dd, |
| | | J = 5.6, 10.2 Hz, 1H), 1.91 (s, 1H), 1.87-1.72 (m, 2H), 1.61-1.31 (m, 1H) |
| I-384 | 807.4 | 10.85 (s, 1H), 8.19 (s, 1H), 8.12-8.08 (m, 1H), 7.73 (d, J = 8.4 Hz, 2H), 7.69 |
| | | (d, J = 9.2 Hz, 1H), 7.55 (dd, J = 2.8, 6.8 Hz, 1H), 7.51 (d, J = 1.6 Hz, 1H), |
| | | 7.31 (t, J = 8.0 Hz, 1H), 7.10-7.04 (m, 3H), 7.00 (d, J = 8.0 Hz, 1H), 4.61 (q, |
| | | J = 6.8 Hz, 2H), 4.52-4.44 (m, 1H), 3.94-3.83 (m, 2H), 3.29-3.22 (m, 8H), |
| | | 3.08-3.00 (m, 6H), 2.90-2.77 (m, 3H), 2.66-2.56 (m, 8H), 2.23 (dq, J = 4.4, |
| | | 12.4 Hz, 1H), 2.02 (td, J = 4.4, 8.8 Hz, 1H), 1.93 (br s, 2H), 1.78 (br d, J = 13.2 |
| | | Hz, 1H), 1.58-1.36 (m, 1H) |
| I-385 | 807.4 | 10.85 (s, 1H), 8.19 (s, 1H), 8.12-8.08 (m, 1H), 7.73 (d, J = 8.4 Hz, 2H), 7.69 |
| | | (d, J = 9.2 Hz, 1H), 7.55 (dd, J = 2.8, 6.8 Hz, 1H), 7.51 (d, J = 1.6 Hz, 1H), |
| | | 7.31 (t, J = 8.0 Hz, 1H), 7.10-7.04 (m, 3H), 7.00 (d, J = 8.0 Hz, 1H), 4.61 (q, |
| | | J = 6.8 Hz, 2H), 4.52-4.44 (m, 1H), 3.94-3.83 (m, 2H), 3.29-3.22 (m, 8H), |
| | | 3.08-3.00 (m, 6H), 2.90-2.77 (m, 3H), 2.66-2.56 (m, 8H), 2.23 (dq, J = 4.4, |
| | | 12.4 Hz, 1H), 2.02 (td, J = 4.4, 8.8 Hz, 1H), 1.93 (br s, 2H), 1.78 (br d, J = 13.2 |
| | | Hz, 1H), 1.58-1.36 (m, 1H) |
| I-386 | 876.4 | 12.20 (d, J = 7.2 Hz, 2H), 8.15-8.06 (m, 2H), 7.69 (s, 1H), 7.67 (s, 1H), 7.63- |
| | | 7.58 (m, 2H), 7.52-7.48 (m, 2H), 7.47-7.43 (m, 4H), 7.00 (d, J = 6.4 Hz, |
| | | 1H), 6.92 (d, J = 6.4 Hz, 1H), 6.43 (s, 2H), 6.18-6.12 (m, 2H), 4.65-4.59 (m, |
| | | 4H), 4.39-4.31 (m, 4H), 3.66-3.57 (m, 4H), 3.18-3.00 (m, 16H), 2.36-2.25 |
| | | (m, 4H) |
| I-388 | 771.6 | 12.02-11.85 (m, 1H), 10.98-10.80 (m, 1H), 7.21-7.13 (m, 1H), 7.00 (d, J = |
| | | 8.4 Hz, 2H), 6.90-6.80 (m, 1H), 6.71-6.63 (m, 1H), 6.59 (br d, J = 8.4 Hz, |
| | | 1H), 6.50-6.44 (m, 1H), 6.16-6.05 (m, 1H), 4.36-4.28 (m, 2H), 3.94-3.87 |
| | | (m, 1H), 3.71 (s, 3H), 3.61 (td, J = 5.6, 11.6 Hz, 2H), 3.29 (s, 2H), 3.26-3.21 |
| | | (m, 4H), 3.17-3.02 (m, 4H), 2.87-2.80 (m, 2H), 2.64-2.60 (m, 4H), 2.58- |
| | | 2.54 (m, 4H), 2.39-2.35 (m, 1H), 2.32-2.21 (m, 2H), 2.07 (d, J = 7.6 Hz, 3H), |
| | | 2.04-1.97 (m, 1H) |
| I-389 | 411.8^{b} | 10.90 (s, 1H), 7.79-7.69 (m, 3H), 7.55 (s, 1H), 7.52 (s, 1H), 7.45-7.37 (m, |
| | | 1H), 7.27 (dd, J = 6.0, 10.4 Hz, 1H), 7.18 (dd, J = 6.2, 10.4 Hz, 1H), 7.09 (br |
| | | d, J = 8.8 Hz, 2H), 6.24-6.13 (m, 2H), 4.40-4.34 (m, 3H), 4.30 (br s, 1H), |
| | | 4.04 (dd, J = 4.8, 12.8 Hz, 1H), 3.66 (br t, J = 5.2 Hz, 1H), 3.59 (br t, J = 5.6 |
| | | Hz, 1H), 3.28 (br s, 3H), 3.25 (br d, J = 4.0 Hz, 4H), 3.04 (br s, 3H), 3.00- |
| | | 2.94 (m, 2H), 2.84-2.79 (m, 2H), 2.76-2.71 (m, 1H), 2.62 (br d, J = 4.8 Hz, |
| | | 5H), 2.59-2.55 (m, 2H), 2.36-2.32 (m, 1H), 2.30-2.17 (m, 2H), 2.04-1.96 |
| | | (m, 1H) |
| I-390 | 822.4 | 10.87 (br s, 1H), 7.77-7.69 (m, 3H), 7.54 (s, 1H), 7.52 (br s, 1H), 7.44-7.34 |
| | | (m, 1H), 7.08 (br d, J = 8.8 Hz, 2H), 7.03-6.97 (m, 2H), 6.22-6.13 (m, 2H), |
| | | 4.39-4.28 (m, 4H), 3.96-3.86 (m, 1H), 3.67-3.56 (m, 2H), 3.29 (s, 2H), 3.28- |
| | | 3.26 (m, 2H), 3.24 (br s, 4H), 3.04 (br s, 3H), 3.01-2.95 (m, 2H), 2.82 (br t, |
| | | J = 7.2 Hz, 2H), 2.62 (br s, 4H), 2.56 (br s, 2H), 2.33 (br d, J = 1.6 Hz, 2H), |
| | | 2.31-2.20 (m, 2H), 2.06-1.96 (m, 1H) |
| I-396 | 860.5 | 11.53-11.41 (m, 1H), 11.08 (s, 1H), 8.18 (s, 1H), 7.69-7.63 (m, 1H), 7.57- |
| | | 7.52 (m, 1H), 7.26-7.21 (m, 1H), 7.07 (s, 1H), 7.03-6.96 (m, 2H), 6.92 (s, |
| | | 1H), 6.90 (d, J = 9.2 Hz, 1H), 6.24 (s, 1H), 5.34 (dd, J = 5.6, 12.4 Hz, 1H), 4.37 |
| | | (s, 2H), 3.63-3.59 (m, 2H), 3.32 (s, 3H), 3.26 (t, J = 7.2 Hz, 5H), 3.18-3.08 |
| | | (m, 3H), 3.00-2.84 (m, 5H), 2.80-2.74 (m, 2H), 2.73-2.67 (m, 1H), 2.65- |
| | | 2.55 (m, 5H), 2.55-2.52 (m, 3H), 2.45-2.41 (m, 2H), 2.33 (d, J = 2.0 Hz, 2H), |
| | | 2.28-2.20 (m, 1H), 2.02-1.89 (m, 5H), 1.70-1.56 (m, 2H), 1.54-1.42 (m, |
| | | 2H) |
| I-401 | 806.8 | 10.90-10.80 (m, 1H), 7.77-7.69 (m, 3H), 7.57-7.50 (m, 2H), 7.46-7.38 (m, |
| | | 1H), 7.33 (t, J = 8.4 Hz, 1H), 7.12-7.04 (m, 3H), 7.03-6.99 (m, 1H), 6.21 (td, |
| | | J = 2.0, 9.6 Hz, 1H), 4.57-4.44 (m, 1H), 4.41-4.31 (m, 2H), 3.94-3.83 (m, |
| | | 2H), 3.29-3.22 (m, 9H), 3.11-3.00 (m, 4H), 2.99-2.89 (m, 3H), 2.87-2.78 |
| | | (m, 3H), 2.65-2.56 (m, 7H), 2.30-2.17 (m, 2H), 2.09-1.99 (m, 1H), 1.93 (br |
| | | d, J = 1.2 Hz, 2H), 1.84-1.74 (m, 1H), 1.60-1.38 (m, 1H) |
| I-402 | 816.7 | 10.73 (s, 1H), 7.81-7.66 (m, 3H), 7.60-7.35 (m, 3H), 7.14-7.00 (m, 3H), |
| | | 6.94 (s, 1H), 6.81 (br d, J = 7.6 Hz, 1H), 6.26-6.04 (m, 2H), 4.42-4.26 (m, |
| | | 4H), 3.94-3.85 (m, 1H), 3.75 (s, 3H), 3.69-3.57 (m, 2H), 3.27 (br s, 8H), |
| | | 3.10-2.93 (m, 5H), 2.83-2.74 (m, 2H), 2.70-2.58 (m, 7H), 2.38-2.25 (m, 2H), |
| | | 2.24-2.12 (m, 1H), 1.89 (td, J = 4.0, 8.0 Hz, 1H) |
| I-405 | 872.4 | 11.09 (s, 1H), 7.75-7.69 (m, 3H), 7.56-7.51 (m, 2H), 7.44-7.37 (m, 1H), |
| | | 7.08 (br d, J = 8.8 Hz, 2H), 6.99-6.95 (m, 2H), 6.93-6.90 (m, 1H), 6.20 (td, |
| | | J = 2.0, 9.5 Hz, 1H), 5.36 (dd, J = 5.2 12.2 Hz, 1H), 4.53-4.44 (m, 1H), 4.35 |
| | | (q, J = 6.8 Hz, 2H), 3.94-3.85 (m, 1H), 3.59 (s, 3H), 3.27 (br s, 3H), 3.24 (br |
| | | s, 3H), 3.03 (br s, 3H), 3.00-2.81 (m, 8H), 2.70-2.55 (m, 6H), 2.02-1.88 (m, |
| | | 4H), 1.87-1.70 (m, 4H), 1.65-1.25 (m, 2H) |
| I-407 | 898.7 | 11.14-11.00 (m, 1H), 8.26-8.18 (m, 1H), 7.79-7.65 (m, 3H), 7.61-7.34 (m, |
| | | 3H), 7.07 (br d, J = 8.4 Hz, 2H), 7.00-6.89 (m, 3H), 6.27-6.08 (m, 2H), 5.36 |
| | | (br dd, J = 5.2, 12.4 Hz, 1H), 4.39-4.26 (m, 4H), 3.65 (br t, J = 5.6 Hz, 1H), |
| | | 3.60-3.57 (m, 4H), 3.52-3.45 (m, 3H), 3.26 (br s, 2H), 3.22 (br s, 4H), 3.01- |
| | | 2.93 (m, 5H), 2.90-2.83 (m, 1H), 2.73-2.62 (m, 2H), 2.54 (br s, 3H), 2.43 |
| | | (br t, J = 6.4 Hz, 2H), 2.33 (br s, 1H), 2.27 (br s, 1H), 2.01-1.93 (m, 1H), |
| | | 1.86-1.77 (m, 2H), 1.69-1.57 (m, 2H), 0.94-0.69 (m, 2H) |
| I-408 | 861.4 | 11.98-11.92 (m, 1H), 11.09 (s, 1H), 7.17 (d, J = 8.4 Hz, 1H), 6.99-6.95 (m, |
| | | 2H), 6.94-6.90 (m, 1H), 6.84 (d, J = 6.4 Hz, 1H), 6.65 (s, 1H), 6.59 (br d, J = |
| | | 8.4 Hz, 1H), 6.49 (d, J = 2.8 Hz, 1H), 6.08 (br s, 1H), 5.37 (br dd, J = 5.6, 12.4 |
| | | Hz, 1H), 4.76-4.66 (m, 4H), 4.37 (br s, 1H), 4.26-4.17 (m, 1H), 4.04 (br s, |
| | | 1H), 3.71 (s, 3H), 3.70-3.66 (m, 1H), 3.59 (s, 3H), 3.23 (br d, J = 2.8 Hz, 6H), |
| | | 3.00-2.94 (m, 3H), 2.94-2.83 (m, 2H), 2.75-2.61 (m, 3H), 2.55 (br s, 4H), |
| | | 2.45-2.42 (m, 3H), 2.34-2.27 (m, 3H), 1.99 (br dd, J = 4.8, 10.8 Hz, 1H), |
| | | 1.86-1.79 (m, 2H) |
| I-411 | 893.3 | 11.09 (d, J = 0.8 Hz, 1H), 7.74 (d, J = 8.4 Hz, 2H), 7.67 (dd, J = 3.2, 12.4 Hz, |
| | | 1H), 7.59-7.53 (m, 2H), 7.51 (d, J = 3.2 Hz, 1H), 7.09 (d, J = 7.6 Hz, 2H), |
| | | 7.01 (dd, J = 4.4, 8.8 Hz, 1H), 6.90 (dd, J = 8.8, 10.4 Hz, 1H), 5.38 (dd, J = |
| | | 5.6, 12.4 Hz, 1H), 4.54-4.43 (m, 1H), 3.96 (d, J = 13.2 Hz, 1H), 3.62 (s, 3H), |
| | | 3.29-3.20 (m, 9H), 3.16-3.09 (m, 3H), 3.03 (s, 3H), 2.96-2.80 (m, 4H), |
| | | 2.76-2.69 (m, 1H), 2.69-2.58 (m, 8H), 2.04-1.89 (m, 3H), 1.83-1.73 (m, 1H), |
| | | 1.63-1.41 (m, 1H) |
| I-412 | 795.3 | 11.95 (br d, J = 6.4 Hz, 1H), 10.85 (s, 1H), 7.32 (t, J = 8.0 Hz, 1H), 7.17 (d, |
| | | J = 8.4 Hz, 1H), 7.05 (d, J = 11.2 Hz, 1H), 7.01 (dd, J = 1.6, 7.6 Hz, 1H), 6.84 |
| | | (d, J = 6.4 Hz, 1H), 6.66 (d, J = 1.6 Hz, 1H), 6.59 (br d, J = 8.4 Hz, 1H), 6.49 |
| | | (d. J = 2.0 Hz, 1H), 6.08 (br s, 1H), 4.76-4.66 (m, 4H), 4.38 (br s, 1H), 4.25- |
| | | 4.17 (m, 1H), 4.04 (br s, 1H), 3.87 (dd, J = 4.4, 11.6 Hz, 1H), 3.71 (s, 3H), 3.70- |
| | | 3.66 (m, 1H), 3.24 (br s, 6H), 3.14-2.99 (m, 4H), 2.84-2.79 (m, 2H), 2.72- |
| | | 2.66 (m, 1H), 2.65-2.60 (m, 5H), 2.59-2.55 (m, 2H), 2.55-2.53 (m, 1H), |
| | | 2.29 (br s, 2H), 2.25-2.17 (m, 1H), 2.18-2.17 (m, 1H), 2.06-2.00 (m, 1H) |
| I-413 | 819.3 | 10.84-10.74 (m, 1H), 7.78-7.69 (m, 3H), 7.57-7.54 (m, 1H), 7.53-7.46 (m, |
| | | 1H), 7.46-7.36 (m, 1H), 7.09 (d, J = 8.8 Hz, 2H), 7.02 (t, J = 8.8 Hz, 1H), |
| | | 6.50-6.43 (m, 2H), 6.23-6.14 (m, 2H), 6.01 (d, J = 7.6 Hz, 1H), 4.37 (d, J = 7.2 |
| | | Hz, 3H), 4.34-4.28 (m, 2H), 3.68-3.58 (m, 2H), 3.30-3.22 (m, 8H), 3.04 (s, |
| | | 3H), 3.01-2.95 (m, 2H), 2.79-2.71 (m, 1H), 2.68-2.64 (m, 2H), 2.60 (t, J = |
| | | 4.0 Hz, 4H), 2.38-2.25 (m, 3H), 2.13-2.06 (m, 1H), 1.92-1.82 (m, 1H) |
| I-414 | 877.4 | 12.15 (s, 1H), 11.12 (s, 1H), 7.46 (d, J = 8.4 Hz, 1H), 7.09 (d, J = 8.0 Hz, 1H), |
| | | 7.05-6.98 (m, 2H), 6.97-6.86 (m, 2H), 6.53 (d, J = 2.4 Hz, 1H), 6.19-6.06 |
| | | (m, 1H), 5.38 (dd, J = 5.6, 12.4 Hz, 1H), 4.39-4.24 (m, 2H), 3.61 (td, J = 5.6, |
| | | 12.0 Hz, 2H), 3.50 (s, 3H), 3.22 (s, 4H), 3.20-3.02 (m, 6H), 3.02-2.83 (m, |
| | | 5H), 2.83-2.52 (m, 6H), 2.40-2.23 (m, 2H), 2.08 (s, 2H), 2.06 (s, 1H), 2.05- |
| | | 1.99 (m, 1H) |
| I-415 | 859.3 | 12.08 (s, 1H), 11.12 (s, 1H), 7.40-7.28 (m, 1H), 7.21-7.10 (m, 1H), 7.04- |
| | | 6.96 (m, 1H), 6.95-6.91 (m, 2H), 6.87 (d, J = 6.0 Hz, 1H), 6.81 (s, 1H), 6.54 |
| | | (d, J = 2.4 Hz, 1H), 6.20-6.06 (m, 1H), 5.37-5.39 (m, 1H), 4.40-4.24 (m, |
| | | 2H), 3.61-3.63 (m, 2H), 3.49 (d, J = 1.6 Hz, 3H), 3.26 (s, 4H), 3.19-2.95 (m, |
| | | 6H), 2.93-2.81 (m, 3H), 2.79-2.68 (m, 2H), 2.68-2.61 (m, 3H), 2.61-2.54 |
| | | (m, 2H), 2.43 (s, 1H), 2.37 (s, 1H), 2.27 (s, 1H), 2.07 (d, J = 8.8 Hz, 3H), |
| | | 2.04-1.96 (m, 1H) |
| I-419 | 787.3 | 11.96 (br s, 1H), 10.99-10.87 (m, 1H), 7.53 (d, J = 7.2 Hz, 1H), 7.24 (d, J = |
| | | 10.8 Hz, 1H), 7.20-7.15 (m, 1H), 6.91-6.81 (m, 1H), 6.67 (s, 1H), 6.63-6.57 |
| | | (m, 1H), 6.17-6.04 (m, 1H), 4.38-4.28 (m, 2H), 4.24-4.14 (m, 1H), 3.72 (s, |
| | | 3H), 3.66-3.57 (m, 2H), 3.25 (br s, 5H), 3.21-2.98 (m, 6H), 2.89-2.72 (m, |
| | | 3H), 2.69-2.55 (m, 7H), 2.41-2.22 (m, 4H), 2.08 (d, J = 7.6 Hz, 3H), 2.03- |
| | | 1.92 (m, 1H) |
| I-420 | 902.3 | 11.11 (s, 1H), 7.80-7.67 (m, 3H), 7.60-7.52 (m, 2H), 7.41 (dd, J = 1.2, 16.2 |
| | | Hz, 1H), 7.09 (br d, J = 8.4 Hz, 2H), 7.05-6.97 (m, 1H), 6.95-6.86 (m, 1H), |
| | | 6.20 (td, J = 2.0, 9.6 Hz, 1H), 5.38 (br dd, J = 5.6, 12.8 Hz, 1H), 4.49 (br t, J = |
| | | 10.0 Hz, 1H), 4.35 (q, J = 7.2 Hz, 2H), 3.90 (br t, J = 11.6 Hz, 1H), 3.62 (s, |
| | | 3H), 3.26 (br s, 3H), 3.20-3.09 (m, 4H), 3.04 (br s, 3H), 2.97-2.84 (m, 4H), |
| | | 2.72 (br d, J = 4.4 Hz, 1H), 2.70-2.61 (m, 8H), 2.09-1.86 (m, 4H), 1.77 (br |
| | | dd, J = 2.8, 13.2 Hz, 1H), 1.59-1.41 (m, 1H), 0.76 (br d, J = 4.4 Hz, 2H), 0.63 |
| | | (br s, 2H) |
| I-423 | 886.5 | 11.32-10.92 (m, 1H), 7.78-7.65 (m, 3H), 7.57-7.46 (m, 2H), 7.45-7.39 (m, |
| | | 1H), 7.10 (br d, J = 8.0 Hz, 2H), 7.02-6.92 (m, 3H), 6.23-6.18 (m, 1H), 5.38 |
| | | (dd, J = 5.2, 12.4 Hz, 1H), 4.54-4.45 (m, 1H), 4.41-4.31 (m, 2H), 3.97-3.84 |
| | | (m, 1H), 3.62 (s, 3H), 3.54-3.40 (m, 2H), 3.27 (br s, 7H), 3.18-3.10 (m, 3H), |
| | | 3.10-2.87 (m, 7H), 2.72-2.63 (m, 8H), 2.08-1.89 (m, 4H), 1.81-1.58 (m, |
| | | 3H), 1.54-1.40 (m, 1H), 1.34-1.18 (m, 2H), 0.99-0.68 (m, 4H) |
| I-424 | 838.3 | 10.92 (s, 1H), 7.81-7.68 (m, 3H), 7.57-7.36 (m, 4H), 7.24 (d, J = 10.8 Hz, |
| | | 1H), 7.09 (br d, J = 8.8 Hz, 2H), 6.27-6.10 (m, 2H), 4.43-4.27 (m, 4H), 4.19 |
| | | (dd, J = 4.8, 12.8 Hz, 1H), 3.70-3.56 (m, 2H), 3.26 (br d, J = 13.6 Hz, 7H), |
| | | 3.09-2.93 (m, 5H), 2.85-2.74 (m, 3H), 2.68-2.55 (m, 7H), 2.39-2.24 (m, |
| | | 3H), 2.02-1.91 (m, 1H) |
| I-427 | 883.3 | 12.08 (br s, 1H), 11.23-10.97 (m, 1H), 7.72 (dd, J = 2.0, 10.8 Hz, 1H), 7.57- |
| | | 7.37 (m, 3H), 7.08-6.95 (m, 5H), 6.94-6.82 (m, 2H), 6.20 (td, J = 2.0, 11.0 |
| | | Hz, 1H), 5.37 (dd, J = 5.6, 12.8 Hz, 1H), 4.48 (br d, J = 6.8 Hz, 3H), 4.36 (q, |
| | | J = 7.2 Hz, 2H), 4.09 (br s, 2H), 3.89 (br d, J = 11.6 Hz, 1H), 3.60 (s, 3H), 3.20 |
| | | (br s, 4H), 3.06-2.84 (m, 7H), 2.83-2.60 (m, 3H), 2.59-2.53 (m, 4H), 2.44 |
| | | (br t, J = 6.8 Hz, 2H), 2.32 (td, J = 7.6, 15.2 Hz, 2H), 1.99-1.74 (m, 6H), |
| | | 1.58-1.38 (m, 1H) |
| I-428 | 869.4 | 12.12-12.04 (m, 1H), 11.16-11.00 (m, 1H), 7.72 (dd, J = 2.0, 10.8 Hz, 1H), |
| | | 7.52 (s, 2H), 7.45-7.39 (m, 1H), 7.08 (d, J = 8.4 Hz, 2H), 7.04-6.96 (m, 4H), |
| | | 6.84 (s, 1H), 6.21 (d, J = 10.8 Hz, 1H), 5.39 (dd, J = 5.2, 12.8 Hz, 1H), 4.48 (s, |
| | | 3H), 4.36 (d, J = 7.2 Hz, 2H), 4.09 (s, 2H), 3.89 (d, J = 13.2 Hz, 1H), 3.62 (s, |
| | | 3H), 3.13 (d, J = 7.6 Hz, 2H), 3.09 (d, J = 2.4 Hz, 1H), 2.96-2.86 (m, 4H), |
| | | 2.74 (d, J = 4.4 Hz, 2H), 2.71-2.64 (m, 8H), 2.61 (s, 1H), 2.36-2.29 (m, 4H), |
| | | 2.03-1.98 (m, 1H), 1.94-1.88 (m, 2H), 1.77 (d, J = 11.2 Hz, 1H), 1.55-1.43 |
| | | (m, 1H) |
| I-430 | 900.5 | 11.10 (s, 1H), 7.76-7.65 (m, 3H), 7.56-7.46 (m, 2H), 7.41 (br d, J = 16.0 Hz, |
| | | 1H), 7.07 (br d, J = 8.4 Hz, 2H), 6.99-6.95 (m, 2H), 6.94-6.89 (m, 1H), 6.19 |
| | | (dd, J = 2.0, 9.6 Hz, 1H), 5.42-5.31 (m, 1H), 4.53-4.43 (m, 1H), 4.40-4.30 |
| | | (m, 2H), 3.95-3.83 (m, 1H), 3.59 (s, 3H), 3.50-3.41 (m, 2H), 3.23 (br d, J = |
| | | 10.4 Hz, 5H), 3.09-2.81 (m, 10H), 2.75-2.63 (m, 2H), 2.56 (br s, 4H), 2.45 |
| | | (br s, 2H), 2.00-1.89 (m, 3H), 1.85-1.73 (m, 3H), 1.65-1.42 (m, 3H), 0.91- |
| | | 0.69 (m, 3H) |
| I-431 | 456.5^{b} | 7.70 (dd, J = 2.0, 11.2 Hz, 1H), 7.50 (d, J = 8.8 Hz, 2H), 7.41 (dd, J = 1.2, 16.4 |
| | | Hz, 1H), 7.04 (br d, J = 8.4 Hz, 2H), 7.00-6.96 (m, 3H), 6.93-6.89 (m, 1H), |
| | | 6.71 (d, J = 2.8 Hz, 1H), 6.20 (td, J = 2.0, 10.4 Hz, 1H), 5.36 (dd, J = 5.6, 12.4 |
| | | Hz, 1H), 4.48 (br d, J = 11.6 Hz, 1H), 4.37-4.32 (m, 2H), 3.88 (br d, J = 11.6 |
| | | Hz, 1H), 3.61 (br d, J = 4.8 Hz, 4H), 3.59 (s, 3H), 3.18 (br s, 4H), 2.99-2.94 |
| | | (m, 3H), 2.91 (br d, J = 7.6 Hz, 2H), 2.81-2.73 (m, 1H), 2.71-2.66 (m, 1H), |
| | | 2.64-2.58 (m, 2H), 2.54 (br s, 2H), 2.43 (br t, J = 6.8 Hz, 3H), 2.00-1.88 (m, |
| | | 3H), 1.86-1.71 (m, 4H), 1.65-1.41 (m, 8H) |
| I-432 | 449.5^{b} | 12.05 (s, 1H), 11.09 (s, 1H), 7.71 (dd, J = 2.0, 10.8 Hz, 1H), 7.51 (d, J = 8.8 |
| | | Hz, 2H), 7.41 (dd, J = 1.2, 16.4 Hz, 1H), 7.07 (d, J = 8.4 Hz, 2H), 6.99 (d, J = |
| | | 5.6 Hz, 3H), 6.96-6.93 (m, 1H), 6.72 (d, J = 2.4 Hz, 1H), 6.20 (td, J = 2.0, |
| | | 10.4 Hz, 1H), 5.38 (dd, J = 5.6, 12.8 Hz, 1H), 4.49 (d, J = 10.8 Hz, 1H), 4.38- |
| | | 4.32 (m, 2H), 3.92-3.85 (m, 1H), 3.64-3.62 (m, 2H), 3.61 (s, 3H), 3.29 (s, |
| | | 2H), 3.24 (s, 4H), 3.16-3.11 (m, 2H), 2.97-2.84 (m, 4H), 2.70-2.63 (m, 8H), |
| | | 2.03-1.97 (m, 1H), 1.95-1.87 (m, 2H), 1.76 (d, J = 11.6 Hz, 1H), 1.70-1.36 |
| | | (m, 9H) |
| I-434 | 897.4 | 12.02-11.97 (m, 1H), 11.10 (br s, 1H), 8.27 (s, 1H), 7.71 (dd, J = 2.0, 10.8 Hz, |
| | | 1H), 7.52 (d, J = 8.4 Hz, 2H), 7.42 (br d, J = 16.8 Hz, 1H), 7.06 (br d, J = 8.4 |
| | | Hz, 2H), 6.99-6.90 (m, 5H), 6.20 (br d, J = 11.2 Hz, 1H), 5.37 (br dd, J = 5.2, |
| | | 12.8 Hz, 1H), 4.52-4.46 (m, 1H), 4.35 (q, J = 6.8 Hz, 2H), 3.89 (br d, J = 14.4 |
| | | Hz, 1H), 3.75 (br s, 2H), 3.60 (s, 3H), 3.55 (br d, J = 6.4 Hz, 2H), 3.20 (br s, |
| | | 4H), 3.10-3.01 (m, 2H), 2.99-2.94 (m, 3H), 2.93-2.89 (m, 2H), 2.82-2.73 |
| | | (m, 1H), 2.55 (br s, 4H), 2.44 (br t, J = 6.4 Hz, 2H), 2.07-1.71 (m, 12H), |
| | | 1.56-1.41 (m, 1H) |
| I-438 | 873.9 | 11.24-10.88 (m, 1H), 8.10 (d, J = 12.0 Hz, 1H), 7.81-7.60 (m, 2H), 7.51- |
| | | 7.46 (m, 2H), 7.39-7.29 (m, 1H), 7.09 (br d, J = 8.4 Hz, 2H), 7.01-6.92 (m, |
| | | 3H), 6.27 (br s, 1H), 5.35 (br dd, J = 5.6, 12.4 Hz, 1H), 4.66-4.58 (m, 2H), |
| | | 4.41-4.31 (m, 2H), 3.69-3.59 (m, 5H), 3.30-3.24 (m, 6H), 3.18-3.06 (m, |
| | | 8H), 2.92-2.84 (m, 1H), 2.72-2.61 (m, 8H), 2.37 (br s, 1H), 2.33 (br s, 1H), |
| | | 2.04-1.93 (m, 1H) |
| I-441 | 771.7 | 11.94 (s, 1H), 10.98-10.88 (m, 1H), 7.21-7.15 (m, 2H), 7.11-7.05 (m, 1H), |
| | | 6.84 (d, J = 4.4 Hz, 1H), 6.67 (s, 1H), 6.60 (d, J = 8.4 Hz, 1H), 6.49 (s, 1H), |
| | | 6.15-6.09 (m, 1H), 4.33 (d, J = 9.2 Hz, 3H), 4.14-4.08 (m, 2H), 3.72 (s, 3H), |
| | | 3.64 (d, J = 5.6 Hz, 2H), 3.62-3.58 (m, 3H), 3.25 (s, 6H), 2.91-2.85 (m, 3H), |
| | | 2.78-2.74 (m, 2H), 2.63 (d, J = 4.8 Hz, 5H), 2.37 (s, 1H), 2.30-2.21 (m, 2H), |
| | | 2.09 (s, 1H), 2.07 (s, 1H), 2.05-1.99 (m, 1H) |
| I-443 | 872.2 | 10.85 (s, 1H), 7.73-7.68 (m, 1H), 7.50 (d, J = 0.8 Hz, 1H), 7.43 (br s, 1H), |
| | | 7.41-7.37 (m, 2H), 7.30 (s, 1H), 7.15 (d, J = 5.6 Hz, 1H), 7.04 (br s, 3H), |
| | | 6.84-6.79 (m, 1H), 6.23-6.17 (m, 1H), 4.49 (br d, J = 11.6 Hz, 1H), 4.38-4.33 |
| | | (m, 2H), 3.87 (br dd, J = 4.4, 11.6 Hz, 2H), 3.30 (s, 4H), 3.27 (br s, 3H), 3.20 |
| | | (br s, 3H), 3.00 (br s, 3H), 2.91 (br d, J = 5.2 Hz, 2H), 2.81 (br s, 2H), 2.62 (br |
| | | s, 6H), 2.32-2.16 (m, 2H), 2.06-1.94 (m, 2H), 1.93-1.83 (m, 2H), 1.78- |
| | | 1.73 (m, 1H), 1.53-1.41 (m, 1H) |
| I-444 | 904.4 | 11.10 (s, 1H), 7.75-7.66 (m, 3H), 7.57-7.46 (m, 2H), 7.41 (dd, J = 1.6, 16.0 |
| | | Hz, 1H), 7.09 (br d, J = 8.0 Hz, 2H), 7.01 (dd, J = 4.8, 8.8 Hz, 1H), 6.90 (dd, |
| | | J = 8.8, 10.8 Hz, 1H), 6.20 (td, J = 2.0, 9.6 Hz, 1H), 5.38 (dd, J = 5.6, 12.4 Hz, |
| | | 1H), 4.53-4.42 (m, 1H), 4.35 (q, J = 6.8 Hz, 2H), 3.93-3.85 (m, 1H), 3.62 (s, |
| | | 3H), 3.51-3.41 (m, 2H), 3.26 (br s, 5H), 3.15-3.10 (m, 2H), 3.08-2.82 (m, |
| | | 7H), 2.72-2.56 (m, 9H), 2.03-1.90 (m, 3H), 1.81-1.72 (m, 1H), 1.67-1.55 |
| | | (m, 2H), 1.54-1.37 (m, 1H), 0.92-0.71 (m, 3H) |
| I-445 | 822.3 | 10.92 (s, 1H), 7.78-7.69 (m, 3H), 7.55-7.53 (m, 1H), 7.52-7.45 (m, 1H), |
| | | 7.45-7.37 (m, 1H), 7.20-7.13 (m, 1H), 7.12-7.05 (m, 3H), 6.23-6.12 (m, |
| | | 2H), 4.40-4.34 (m, 3H), 4.30 (br s, 1H), 4.14-4.07 (m, 1H), 3.66 (br t, J = |
| | | 5.2 Hz, 1H), 3.59 (br t, J = 5.6 Hz, 1H), 3.26 (br d, J = 4.0 Hz, 6H), 3.08-3.01 |
| | | (m, 3H), 3.00-2.95 (m, 2H), 2.90-2.85 (m, 2H), 2.82-2.70 (m, 2H), 2.63 (br |
| | | d, J = 5.6 Hz, 5H), 2.59 (br d, J = 7.6 Hz, 2H), 2.34 (br d, J = 1.6 Hz, 1H), |
| | | 2.30-2.21 (m, 2H), 2.07-1.98 (m, 1H) |
| I-449 | 450.9^{b} | 12.08 (br s, 1H), 11.09 (s, 1H), 7.70-7.62 (m, 1H), 7.51 (br d, J = 8.4 Hz, 3H), |
| | | 7.05 (br d, J = 8.4 Hz, 3H), 6.98-6.90 (m, 3H), 6.82 (br s, 1H), 5.40-5.33 (m, |
| | | 1H), 4.47 (br s, 3H), 4.12-4.03 (m, 2H), 4.00-3.90 (m, 1H), 3.59 (s, 3H), 3.20 |
| | | (br s, 7H), 3.13-3.07 (m, 1H), 2.96 (br d, J = 7.6 Hz, 2H), 2.91-2.83 (m, 3H), |
| | | 2.58 (br s, 3H), 2.54 (br s, 4H), 2.43 (br s, 2H), 2.39-2.24 (m, 3H), 2.01-1.90 |
| | | (m, 3H), 1.85-1.76 (m, 3H) |
| I-450^{d} | 924.8 | 12.23-11.85 (m, 1H), 11.28-10.92 (m, 1H), 8.42-8.32 (m, 3H), 7.75 (s, 1H), |
| | | 7.52 (d, J = 8.8 Hz, 2H), 7.45-7.35 (m, 1H), 7.10-7.04 (m, 2H), 7.02-6.96 |
| | | (m, 2H), 6.94-6.86 (m, 2H), 6.83 (d, J = 3.0 Hz, 1H), 6.31-6.09 (m, 1H), |
| | | 5.48-5.22 (m, 1H), 4.50-4.46 (m, 2H), 4.39-4.33 (m, 2H), 4.12-4.04 (m, 2H), |
| | | 3.90-3.85 (m, 1H), 3.65 (s, 3H), 3.23 (br s, 6H), 3.19 (br s, 2H), 3.10-3.02 |
| | | (m, 2H), 2.97-2.87 (m, 4H), 2.72 (br s, 6H), 2.65-2.57 (m, 4H), 2.05-1.67 |
| | | (m, 8H), 1.54-1.41 (m, 1H) |
| I-457 | 768.2 | 12.63-11.50 (m, 1H), 11.26-10.20 (m, 1H), 7.23-7.11 (m, 1H), 7.06 (s, 1H), |
| | | 6.90-6.79 (m, 1H), 6.67 (d, J = 2.0 Hz, 1H), 6.60 (d, J = 8.4 Hz, 1H), 6.51- |
| | | 6.43 (m, 3H), 6.17-6.07 (m, 1H), 6.05-5.96 (m, 1H), 4.36-4.27 (m, 3H), |
| | | 3.75-3.69 (m, 3H), 3.66-3.58 (m, 2H), 3.24 (s, 4H), 3.21-2.88 (m, 6H), 2.72 |
| | | (s, 1H), 2.71-2.62 (m, 3H), 2.60 (d, J = 4.4 Hz, 4H), 2.57-2.53 (m, 1H), 2.44 |
| | | (d, J = 4.8 Hz, 1H), 2.41-2.36 (m, 1H), 2.34-2.22 (m, 1H), 2.14-2.06 (m, |
| | | 4H), 1.91-1.81 (m, 1H) |
| I-458 | 767.8 | 11.94 (br s, 1H), 10.84 (s, 1H), 7.31 (t, J = 8.0 Hz, 1H), 7.20-7.14 (m, 1H), |
| | | 7.09-6.96 (m, 2H), 6.91-6.79 (m, 1H), 6.66 (s, 1H), 6.59 (br d, J = 8.4 Hz, |
| | | 1H), 6.48 (br s, 1H), 6.14-6.04 (m, 1H), 4.38-4.28 (m, 2H), 3.87 (dd, J = 4.8, |
| | | 12.0 Hz, 1H), 3.72 (s, 3H), 3.65-3.53 (m, 5H), 3.25-3.04 (m, 6H), 3.00 (br d, |
| | | J = 11.2 Hz, 2H), 2.93-2.85 (m, 2H), 2.82-2.71 (m, 2H), 2.67-2.55 (m, 4H), |
| | | 2.36 (br d, J = 3.2 Hz, 1H), 2.29-2.16 (m, 2H), 2.07 (d, J = 7.6 Hz, 3H), 2.02 |
| | | (td, J = 4.4, 8.8 Hz, 1H), 1.11 (d, J = 5.6 Hz, 3H) |
| I-459 | 767.4 | 11.94 (br s, 1H), 10.84 (s, 1H), 7.32 (t, J = 8.0 Hz, 1H), 7.20-7.15 (m, 1H), |
| | | 7.09-7.00 (m, 2H), 6.90-6.81 (m, 1H), 6.67 (s, 1H), 6.60 (br d, J = 8.4 Hz, |
| | | 1H), 6.52-6.48 (m, 1H), 6.14-6.07 (m, 1H), 4.34-4.29 (m, 2H), 3.87 (dd, |
| | | J = 4.8, 11.8 Hz, 1H), 3.72 (s, 3H), 3.64-3.56 (m, 4H), 3.16-3.01 (m, 6H), 2.95 |
| | | (br d, J = 9.6 Hz, 2H), 2.88-2.73 (m, 3H), 2.67 (ddd, J = 5.4, 12.2, 17.4 Hz, |
| | | 5H), 2.53 (br d, J = 4.4 Hz, 1H), 2.36 (br d, J = 2.8 Hz, 1H), 2.28-2.17 (m, |
| | | 2H), 2.07 (d, J = 7.6 Hz, 3H), 2.02 (br dd, J = 4.4, 8.8 Hz, 1H), 1.14 (br d, J = |
| | | 4.4 Hz, 3H) |
| I-460 | 779.7 | 11.91 (br s, 1H), 10.83 (s, 1H), 7.30 (t, J = 8.0 Hz, 1H), 7.18-7.08 (m, 1H), |
| | | 7.06-6.96 (m, 2H), 6.90-6.82 (m, 1H), 6.55-6.43 (m, 3H), 6.15-6.07 (m, |
| | | 1H), 4.37-4.26 (m, 4H), 3.86 (dd, J = 4.8, 11.6 Hz, 1H), 3.69 (s, 3H), 3.61 (td, |
| | | J = 6.0, 11.6 Hz, 2H), 3.23-2.96 (m, 6H), 2.75-2.69 (m, 2H), 2.68-2.61 (m, |
| | | 3H), 2.48-2.43 (m, 5H), 2.40-2.31 (m, 2H), 2.28-2.16 (m, 2H), 2.07 (d, J = |
| | | 7.2 Hz, 3H), 2.04-1.98 (m, 1H), 1.83 (br s, 3H) |
| I-462 | 892.4 | 11.11 (s, 1H), 7.71 (dd, J = 2.0, 8.0 Hz, 1H), 7.52 (d, J = 1.2 Hz, 1H), 7.46- |
| | | 7.31 (m, 3H), 7.14 (s, 1H), 7.05 (d, J = 8.4 Hz, 1H), 7.02-6.90 (m, 3H), 6.19 |
| | | (d, J = 8.4 Hz, 1H), 5.38 (dd, J = 5.2, 12.4 Hz, 1H), 4.48 (d, J = 11.6 Hz, 1H), |
| | | 4.41-4.26 (m, 2H), 3.95-3.81 (m, 1H), 3.61 (s, 3H), 3.30 (s, 3H), 3.24-3.18 |
| | | (m, 3H), 3.14 (s, 3H), 2.99 (s, 4H), 2.97-2.85 (m, 4H), 2.84-2.52 (m, 9H), |
| | | 2.05-1.96 (m, 1H), 1.96-1.80 (m, 2H), 1.74 (d, J = 11.2 Hz, 1H), 1.59-1.35 |
| | | (m, 1H) |
| I-466 | 889.2 | 11.16-11.01 (m, 1H), 8.10 (d, J = 10.8 Hz, 1H), 7.73-7.64 (m, 2H), 7.46 (br |
| | | d, J = 8.0 Hz, 2H), 7.39 (dd, J = 3.2, 6.4 Hz, 1H), 7.11-7.05 (m, 3H), 7.01 (d, |
| | | J = 8.0 Hz, 1H), 6.95-6.86 (m, 1H), 5.39-5.27 (m, 1H), 4.66-4.58 (m, 2H), |
| | | 4.49 (br d, J = 10.4 Hz, 1H), 4.03-3.84 (m, 1H), 3.34 (s, 3H), 3.24 (br s, 5H), |
| | | 3.14-3.00 (m, 8H), 2.94-2.83 (m, 2H), 2.71-2.62 (m, 4H), 2.54 (br d, J = |
| | | 4.8 Hz, 4H), 2.40-2.34 (m, 2H), 2.04-1.74 (m, 7H), 1.62-1.41 (m, 1H) |
| I-469 | 441.2 [(M + H)/2]⁺ | 11.18-10.99 (m, 1H), 8.15-8.09 (m, 1H), 7.70 (d, J = 2.0 Hz, 1H), 7.58-7.53 |
| | | (m, 1H), 7.11 (dd, J = 2.4, 8.4 Hz, 1H), 7.03-6.93 (m, 3H), 6.87 (s, 1H), 6.68- |
| | | 6.53 (m, 3H), 6.08-5.97 (m, 1H), 5.39 (dd, J = 5.6, 12.4 Hz, 1H), 4.65- 4.62 |
| | | (m, 1H), 4.35-4.11 (m, 2H), 3.71 (s, 3H), 3.67-3.58 (m, 5H), 3.28 (br s, 5H), |
| | | 3.23 (br d, J = 8.0 Hz, 3H), 3.17-3.09 (m, 4H), 3.00 (s, 6H), 2.92-2.86 (m, |
| | | 1H), 2.79-2.72 (m, 1H), 2.68 (br s, 6H), 2.64-2.61 (m, 1H), 2.37-2.33 (m, |
| | | 1H), 2.28 (br d, J = 1.6 Hz, 1H), 2.06-1.96 (m, 1H) |
| I-470 | 922.8 | 11.54-10.81 (m, 1H), 8.21-7.97 (m, 1H), 7.69 (s, 1H), 7.59-7.48 (m, 1H), |
| | | 7.12 (br d, J = 8.4 Hz, 1H), 7.02-6.92 (m, 3H), 6.87 (d, J = 1.6 Hz, 1H), 6.55 |
| | | (s, 3H), 6.04-5.92 (m, 1H), 5.40-5.28 (m, 1H), 4.63 (t, J = 6.8 Hz, 2H), |
| | | 4.36-4.12 (m, 2H), 3.73-3.67 (m, 4H), 3.62 (s, 3H), 3.52 (br d, J = 6.0 Hz, 3H), |
| | | 3.22-3.04 (m, 6H), 3.00 (s, 6H), 2.94-2.77 (m, 2H), 2.59 (br s, 9H), 2.33- |
| | | 2.23 (m, 2H), 2.03-1.95 (m, 1H), 0.65-0.51 (m, 1H), 0.13-0.02 (m, 2H), −0.21- |
| | | 0.32 (m, 2H) |
| I-471 | 838.2 | 10.92 (s, 1H), 7.78-7.67 (m, 3H), 7.54 (s, 1H), 7.53-7.44 (m, 1H), 7.44-7.37 |
| | | (m, 1H), 7.34 (s, 1H), 7.14 (d, J = 7.6 Hz, 1H), 7.08 (br d, J = 8.4 Hz, 2H), |
| | | 6.23-6.11 (m, 2H), 4.39-4.33 (m, 3H), 4.29 (br s, 1H), 4.23 (dd, J = 5.2, 12.4 Hz, |
| | | 1H), 3.68-3.56 (m, 2H), 3.29-3.21 (m, 8H), 3.04 (br s, 3H), 3.00-2.93 (m, |
| | | 2H), 2.86 (br t, J = 7.2 Hz, 2H), 2.83-2.72 (m, 1H), 2.62 (br d, J = 4.4 Hz, |
| | | 5H), 2.58-2.53 (m, 1H), 2.39-2.31 (m, 2H), 2.30-2.22 (m, 1H), 2.04-1.88 |
| | | (m, 1H) |
| I-472 | 819.6 | 10.84 (s, 1H), 7.79-7.66 (m, 3H), 7.60-7.30 (m, 3H), 7.16 (d, J = 8.0 Hz, |
| | | 1H), 7.09 (br d, J = 8.4 Hz, 2H), 6.94 (d, J = 11.6 Hz, 1H), 6.28-6.08 (m, 2H), |
| | | 4.42-4.26 (m, 4H), 4.02 (dd, J = 4.8, 12.4 Hz, 1H), 3.66 (br t, J = 5.6 Hz, 1H), |
| | | 3.59 (br t, J = 5.6 Hz, 1H), 3.30-3.22 (m, 8H), 3.07-2.93 (m, 5H), 2.80-2.71 |
| | | (m, 3H), 2.64-2.55 (m, 6H), 2.34 (br s, 1H), 2.30-2.16 (m, 5H), 2.00-1.90 |
| | | (m, 1H) |
| I-473 | 901.4 | 12.14-12.00 (m, 1H), 11.15-11.01 (m, 1H), 7.72 (dd, J = 2.0, 10.8 Hz, 1H), |
| | | 7.52 (d, J = 8.8 Hz, 2H), 7.47-7.37 (m, 1H), 7.10-7.03 (m, 2H), 7.02-6.96 |
| | | (m, 2H), 6.93-6.86 (m, 1H), 6.85-6.80 (m, 1H), 6.29-6.10 (m, 1H), 5.43- |
| | | 5.33 (m, 1H), 4.55-4.42 (m, 3H), 4.36 (q, J = 6.8 Hz, 2H), 4.16-4.04 (m, 2H), |
| | | 3.89 (br d, J = 12.4 Hz, 1H), 3.65-3.58 (m, 3H), 3.18 (br s, 4H), 3.05-2.84 |
| | | (m, 7H), 2.73-2.60 (m, 3H), 2.58-2.54 (m, 3H), 2.48-2.44 (m, 3H), 2.35- |
| | | 2.30 (m, 2H), 1.98-1.87 (m, 3H), 1.85-1.74 (m, 3H), 1.56-1.42 (m, 1H) |
| I-474 | 915.1 | 11.96-11.89 (m, 1H), 11.09 (s, 1H), 7.75-7.67 (m, 1H), 7.51 (d, J = 8.4 Hz, |
| | | 2H), 7.45-7.37 (m, 1H), 7.04 (br d, J = 8.8 Hz, 3H), 7.00-6.95 (m, 2H), |
| | | 6.91-6.86 (m, 1H), 6.23-6.17 (m, 1H), 5.40-5.31 (m, 1H), 4.52-4.46 (m, 1H), |
| | | 4.35 (br d, J = 7.2 Hz, 2H), 3.93-3.84 (m, 1H), 3.60 (s, 3H), 3.16 (br s, 4H), |
| | | 3.04 (d, J = 1.6 Hz, 4H), 2.94-2.80 (m, 2H), 2.99-2.80 (m, 5H), 2.78 (br s, |
| | | 2H), 2.61 (br s, 1H), 2.54 (br d, J = 3.6 Hz, 3H), 2.47-2.43 (m, 2H), 1.97- |
| | | 1.72 (m, 7H), 1.53-1.42 (m, 1H), 0.79-0.72 (m, 2H), 0.61 (br s, 2H) |
| I-479 | 815.2 | 11.65-11.36 (m, 1H), 11.17-10.92 (m, 1H), 8.14-7.92 (m, 1H), 7.73-7.60 |
| | | (m, 1H), 7.35-7.14 (m, 1H), 6.97 (br dd, J = 4.0, 14.0 Hz, 3H), 6.88-6.77 (m, |
| | | 1H), 6.70-6.46 (m, 2H), 6.21-5.97 (m, 1H), 5.46-5.34 (m, 1H), 4.66-4.55 |
| | | (m, 1H), 4.40-4.29 (m, 1H), 3.92-3.74 (m, 1H), 3.70 (br d, J = 3.6 Hz, 1H), |
| | | 3.66-3.55 (m, 5H), 3.28-3.03 (m, 11H), 3.01-2.81 (m, 2H), 2.75-2.58 (m, |
| | | 8H), 2.38-2.25 (m, 2H), 2.08-1.91 (m, 2H) |
| I-480 | 832.1 | 12.48-11.70 (m, 1H), 11.32-10.42 (m, 1H), 7.75 (s, 1H), 7.56-7.49 (m, 2H), |
| | | 7.46-7.38 (m, 1H), 7.10-6.95 (m, 4H), 6.83 (d, J = 3.2 Hz, 1H), 6.50-6.43 |
| | | (m, 2H), 6.23-6.16 (m, 1H), 6.04-5.96 (m, 1H), 4.54-4.44 (m, 3H), 4.39- |
| | | 4.33 (m, 2H), 4.31-4.24 (m, 1H), 4.15-4.01 (m, 2H), 3.93-3.85 (m, 1H), |
| | | 3.22 (s, 4H), 3.10-3.01 (m, 1H), 2.96-2.89 (m, 2H), 2.81-2.69 (m, 2H), |
| | | 2.69-2.66 (m, 2H), 2.65 (s, 1H), 2.61 (s, 4H), 2.57 (s, 1H), 2.56-2.54 (m, 2H), |
| | | 2.38-2.26 (m, 3H), 2.16-2.04 (m, 1H), 1.97-1.82 (m, 3H), 1.81-1.74 (m, |
| | | 1H), 1.57-1.42 (m, 1H) |
| I-481 | 765.4 | 12.10-11.80 (m, 1H), 11.08-10.65 (m, 1H), 7.28 (t, J = 8.4 Hz, 1H), 7.21- |
| | | 7.16 (m, 1H), 7.05-6.95 (m, 2H), 6.90-6.81 (m, 1H), 6.52 (d, J = 2.4 Hz, 1H), |
| | | 6.45-6.36 (m, 2H), 6.20-6.07 (m, 1H), 4.39-4.28 (m, 2H), 3.89-3.83 (m, |
| | | 1H), 3.78-3.71 (m, 5H), 3.66-3.51 (m, 5H), 3.25-3.00 (m, 7H), 2.72-2.59 |
| | | (m, 4H), 2.41-2.32 (m, 2H), 2.30-2.17 (m, 2H), 2.08 (d, J = 8.4 Hz, 3H), |
| | | 2.04-1.97 (m, 1H), 1.60-1.20 (m, 3H) |
| I-482 | 889.4 | 12.02 (s, 1H), 11.16 (s, 1H), 7.23 (d, J = 8.4 Hz, 1H), 7.07-6.96 (m, 3H), 6.94- |
| | | 6.88 (m, 1H), 6.72 (s, 1H), 6.65 (d, J = 8.4 Hz, 1H), 6.55 (s, 1H), 6.16 (s, 1H), |
| | | 5.43 (dd, J = 5.6, 12.4 Hz, 1H), 4.50-4.38 (m, 2H), 3.95-3.86 (m, 2H), 3.78 |
| | | (s, 3H), 3.76-3.69 (m, 2H), 3.66 (s, 3H), 3.52-3.44 (m, 2H), 3.29 (s, 6H), |
| | | 3.10 (d, J = 9.6 Hz, 1H), 3.07-2.98 (m, 4H), 2.97-2.91 (m, 1H), 2.76 (dd, |
| | | J = 4.4, 12.8 Hz, 1H), 2.70 (s, 1H), 2.67-2.58 (m, 5H), 2.50 (t, J = 6.4 Hz, 2H), |
| | | 2.43 (d, J = 2.0 Hz, 1H), 2.31 (d, J = 2.4 Hz, 1H), 2.07-2.01 (m, 1H), 1.93- |
| | | 1.84 (m, 2H), 1.72-1.57 (m, 4H), 1.30 (s, 1H) |
| I-483 | 779.2 | 11.95 (s, 1H), 10.88 (s, 1H), 7.61-7.32 (m, 1H), 7.24-7.02 (m, 3H), 6.95- |
| | | 6.76 (m, 1H), 6.72-6.63 (m, 1H), 6.58 (d, J = 8.0 Hz, 1H), 6.54-6.39 (m, 1H), |
| | | 6.18-5.97 (m, 1H), 4.36-4.26 (m, 2H), 4.02-3.86 (m, 2H), 3.70 (s, 3H), 3.60- |
| | | 3.63 (m, 3H), 3.30-2.99 (m, 12H), 2.74-2.64 (m, 1H), 2.54 (s, 3H), 2.44- |
| | | 2.21 (m, 3H), 2.14-1.98 (m, 4H), 1.95-1.66 (m, 4H) |
| I-484 | 804.8 | 1.97-2.06 (m, 1 H), 2.17-2.30 (m, 2 H), 2.35 (br d, J = 2.8 Hz, 1 H), 2.55- |
| | | 2.72 (m, 8 H), 2.77-2.84 (m, 2 H), 2.97-3.14 (m, 6 H), 3.18-3.26 (m, 6 H), |
| | | 3.57-3.69 (m, 2 H), 3.87 (dd, J = 12.0, 4.8 Hz, 1 H), 4.30-4.40 (m, 2 H), 4.59- |
| | | 4.66 (m, 2 H), 6.14 (br s, 1 H), 6.85 (br s, 1 H), 6.91-7.02 (m, 2 H), 7.06 (br |
| | | d, J = 9.2 Hz, 3 H), 7.32 (t, J = 8.0 Hz, 1 H), 7.48-7.55 (m, 2 H), 7.65-7.72 |
| | | (m, 1 H), 8.08-8.14 (m, 1 H), 10.85 (br s, 1 H), 12.05-12.19 (m, 1 H) |
| I-489 | 787.3 | 11.94 (br s, 1H), 10.93 (s, 1H), 7.39-7.31 (m, 1H), 7.20-7.10 (m, 2H), 6.91- |
| | | 6.80 (m, 1H), 6.66 (s, 1H), 6.59 (br d, J = 8.4 Hz, 1H), 6.48 (br s, 1H), 6.16- |
| | | 6.04 (m, 1H), 4.32 (br d, J = 9.6 Hz, 2H), 4.23 (br dd, J = 5.2, 12.4 Hz, 1H), |
| | | 3.71 (s, 3H), 3.61 (td, J = 5.6, 11.2 Hz, 2H), 3.24 (br s, 6H), 3.14-3.03 (m, |
| | | 4H), 2.90-2.84 (m, 2H), 2.83-2.72 (m, 2H), 2.62 (br d, J = 4.8 Hz, 4H), 2.56 |
| | | (br s, 2H), 2.35 (br d, J = 8.8 Hz, 2H), 2.31-2.21 (m, 2H), 2.07 (br d, J = 7.6 |
| | | Hz, 2H), 2.02-1.97 (m, 1H) |
| I-490 | 818.5 | 10.84 (s, 1H), 7.76-7.69 (m, 3H), 7.54 (s, 1H), 7.53-7.45 (m, 1H), 7.44-7.36 |
| | | (m, 1H), 7.14 (t, J = 8.0 Hz, 1H), 7.08 (br d, J = 8.4 Hz, 2H), 6.91 (d, J = 8.0 |
| | | Hz, 1H), 6.22-6.13 (m, 2H), 4.42-4.27 (m, 4H), 4.07 (dd, J = 4.8, 12.0 Hz, |
| | | 1H), 3.70-3.55 (m, 2H), 3.29-3.20 (m, 8H), 3.04 (br s, 3H), 3.02-2.92 (m, |
| | | 3H), 2.83-2.77 (m, 2H), 2.76-2.68 (m, 1H), 2.62 (br s, 4H), 2.59-2.52 (m, |
| | | 3H), 2.37-2.26 (m, 2H), 2.20 (br dd, J = 4.0, 12.8 Hz, 1H), 2.15 (d, J = 1.6 |
| | | Hz, 3H), 2.02-1.92 (m, 1H) |
| I-500 | 907.9 | 11.10 (s, 1H), 7.71 (dd, J = 2.0, 10.4 Hz, 1H), 7.54 (s, 1H), 7.43-7.28 (m, 3H), |
| | | 7.13 (d, J = 2.0 Hz, 1H), 7.07-6.98 (m, 2H), 6.94-6.87 (m, 1H), 6.23-6.08 |
| | | (m, 2H), 5.38 (dd, J = 5.2, 12.6 Hz, 1H), 4.38-4.27 (m, 4H), 3.66-3.54 (m, |
| | | 6H), 3.22-3.09 (m, 6H), 3.02-2.93 (m, 5H), 2.90-2.83 (m, 1H), 2.76-2.57 |
| | | (m, 10H), 2.35-2.24 (m, 2H), 2.07-1.95 (m, 1H) |
| I-501 | 835.7 | 11.99-11.88 (m, 1H), 10.42 (s, 1H), 8.15-8.06 (m, 1H), 7.72 (s, 1H), 7.42- |
| | | 7.32 (m, 1H), 7.22-7.11 (m, 3H), 6.91-6.80 (m, 1H), 6.66 (s, 1H), 6.62-6.56 |
| | | (m, 1H), 6.51-6.44 (m, 1H), 6.13-6.06 (m, 1H), 4.66-4.59 (m, 2H), 4.37- |
| | | 4.28 (m, 2H), 3.79 (t, J = 6.4 Hz, 2H), 3.73-3.69 (m, 3H), 3.65 (br t, J = 5.6 |
| | | Hz, 1H), 3.61-3.58 (m, 1H), 3.24 (br s, 6H), 3.15-3.03 (m, 6H), 2.85-2.79 |
| | | (m, 2H), 2.72-2.68 (m, 2H), 2.62 (br d, J = 5.6 Hz, 4H), 2.60-2.55 (m, 2H), |
| | | 2.35-2.25 (m, 2H) |
| I-502 | 846.3 | 11.96-11.87 (m, 1H), 10.84 (s, 1H), 8.14-8.09 (m, 1H), 7.72-7.64 (m, 1H), |
| | | 7.27 (t, J = 8.0 Hz, 1H), 7.12 (d, J = 8.0 Hz, 1H), 7.05 (d, J = 11.2 Hz, 1H), |
| | | 7.00 (d, J = 8.0 Hz, 1H), 6.87-6.77 (m, 1H), 6.44 (d, J = 2.0 Hz, 1H), 6.17- |
| | | 6.04 (m, 3H), 4.67-4.56 (m, 2H), 4.38-4.26 (m, 2H), 3.95 (s, 3H), 3.87 (dd, |
| | | J = 4.8, 12.0 Hz, 1H), 3.70-3.66 (m, 3H), 3.66-3.58 (m, 2H), 3.47 (s, 4H), |
| | | 3.23-2.92 (m, 9H), 2.75-2.59 (m, 5H), 2.53 (s, 1H), 2.33 (d, J = 1.6 Hz, 1H), |
| | | 2.30-2.16 (m, 2H), 2.07-1.97 (m, 1H) |
| I-503 | 416.2^{b} | 1.8-1.9 (m, 1 H), 2.1-2.2 (m, 2 H), 2.3-2.3 (m, 2 H), 2.6 (br d, J = 4.0 Hz, 5 |
| | | H), 2.6-2.7 (m, 3 H), 3.0-3.1 (m, 6 H), 3.2 (br d, J = 3.2 Hz, 6 H), 3.6 (br t, |
| | | J = 5.6 Hz, 2 H), 3.6-3.7 (m, 1 H), 3.7-3.7 (m, 3 H), 3.7-3.7 (m, 3 H), 3.7- |
| | | 3.7 (m, 1 H), 4.3-4.3 (m, 2 H), 4.3-4.4 (m, 1 H), 4.6-4.6 (m, 2 H), 5.7-5.8 |
| | | (m, 1 H), 6.1-6.2 (m, 1 H), 6.5-6.5 (m, 3 H), 6.6-6.6 (m, 2 H), 6.6-6.7 (m, |
| | | 1 H), 6.8-6.9 (m, 1 H), 7.0-7.0 (m, 1 H), 7.1-7.2 (m, 1 H), 7.6-7.7 (m, 1 |
| | | H), 8.1-8.1 (m, 1 H), 8.3-8.4 (m, 1 H), 10.7-10.8 (m, 1 H), 10.8-10.8 (m, |
| | | 1 H), 11.9-12.0 (m, 1 H) |
| I-504 | 805.1 | 12.17 (d, J = 4.8 Hz, 1H), 10.85 (s, 1H), 8.46-8.40 (m, 1H), 8.15-8.08 (m, |
| | | 1H), 7.89-7.82 (m, 1H), 7.72-7.66 (m, 1H), 7.32 (t, J = 8.0 Hz, 1H), 7.08- |
| | | 6.94 (m, 4H), 6.85 (s, 1H), 6.18 (d, J = 8.4 Hz, 1H), 4.63 (q, J = 7.2 Hz, 2H), |
| | | 4.41-4.31 (m, 2H), 3.88 (dd, J = 4.8, 12.0 Hz, 1H), 3.68-3.59 (m, 2H), 3.57 |
| | | (s, 3H), 3.28-3.15 (m, 3H), 3.15-2.92 (m, 6H), 2.87-2.78 (m, 2H), 2.73- |
| | | 2.62 (m, 2H), 2.58 (s, 6H), 2.38-2.33 (m, 1H), 2.31-2.18 (m, 2H), 2.08-1.98 |
| | | (m, 1H) |
| I-505 | 840.5 | 11.94 (s, 1H), 11.17-11.01 (m, 1H), 8.13 (dd, J = 1.2, 4.8 Hz, 1H), 7.60-7.47 |
| | | (m, 1H), 7.20 (d, J = 8.4 Hz, 1H), 7.02-6.87 (m, 5H), 6.69 (d, J = 2.0 Hz, 1H), |
| | | 6.66-6.58 (m, 2H), 6.53-6.46 (m, 1H), 6.15 (br s, 1H), 5.49-5.33 (m, 1H), |
| | | 4.37 (br s, 2H), 3.78 (t, J = 6.0 Hz, 2H), 3.73 (s, 3H), 3.66-3.57 (m, 3H), |
| | | 3.30-3.24 (m, 5H), 3.22-3.06 (m, 7H), 2.71-2.58 (m, 8H), 2.43-2.33 (m, 3H), |
| | | 2.06-1.97 (m, 1H) |
| I-507 | 442.2^{b} | 11.98-11.83 (m, 1H), 11.16-11.03 (m, 1H), 7.71 (dd, J = 2.0, 10.4 Hz, 1H), |
| | | 7.52 (d, J = 8.8 Hz, 2H), 7.41 (dd, J = 1.2, 16.0 Hz, 1H), 7.07 (br d, J = 8.4 Hz, |
| | | 3H), 7.00-6.92 (m, 4H), 6.20 (td, J = 2.0, 10.4 Hz, 1H), 5.37 (dd, J = 5.6, 12.4 |
| | | Hz, 1H), 4.49 (br d, J = 10.4 Hz, 1H), 4.40-4.31 (m, 2H), 3.89 (br d, J = 12.4 |
| | | Hz, 1H), 3.61 (s, 3H), 3.24 (br s, 4H), 3.19-3.07 (m, 4H), 3.04 (d, J = 1.6 Hz, |
| | | 3H), 2.99-2.83 (m, 4H), 2.78-2.72 (m, 1H), 2.71-2.60 (m, 8H), 2.05-1.97 |
| | | (m, 1H), 1.96-1.84 (m, 2H), 1.82-1.72 (m, 1H), 1.58-1.38 (m, 1H), 0.75 (br |
| | | t, J = 5.2 Hz, 2H), 0.61 (br s, 2H) |
| I-512 | 782.4 | 10.83 (s, 1H), 8.30-8.12 (m, 1H), 7.77-7.66 (m, 1H), 7.52-7.34 (m, 2H), |
| | | 7.29 (t, J = 8.0 Hz, 1H), 7.09-6.98 (m, 2H), 6.72 (dd, J = 6.4, 16.4 Hz, 1H), |
| | | 6.25-6.14 (m, 1H), 6.06 (s, 1H), 4.36 (q, J = 6.4 Hz, 2H), 4.31-4.21 (m, 2H), |
| | | 3.95-3.71 (m, 4H), 3.63 (t, J = 5.6 Hz, 1H), 3.56 (t, J = 5.6 Hz, 1H), 3.28 (s, |
| | | 3H), 3.22-3.06 (m, 2H), 3.04 (s, 3H), 3.00-2.91 (m, 2H), 2.76 (t, J = 10.8 Hz, |
| | | 2H), 2.70-2.59 (m, 1H), 2.59-2.51 (m, 1H), 2.33 (d, J = 9.6 Hz, 4H), 2.29- |
| | | 2.13 (m, 2H), 2.08-1.98 (m, 1H), 1.92 (d, J = 10.8 Hz, 2H), 1.83-1.59 (m, |
| | | 6H) |
| I-522 | 859.2 | 11.01-10.72 (m, 1H), 7.80-7.63 (m, 3H), 7.58-7.36 (m, 3H), 7.13-6.93 (m, |
| | | 5H), 6.24-6.11 (m, 2H), 4.42-4.33 (m, 3H), 4.32-4.27 (m, 1H), 3.85-3.76 |
| | | (m, 1H), 3.67-3.58 (m, 2H), 3.46-3.40 (m, 4H), 3.28 (s, 3H), 3.25 (s, 3H), |
| | | 3.09-3.02 (m, 3H), 3.01-2.96 (m, 2H), 2.70 (d, J = 5.2 Hz, 4H), 2.66 (s, 2H), |
| | | 2.34 (d, J = 2.0 Hz, 2H), 2.32-2.24 (m, 2H), 2.22 (s, 1H), 2.04-1.96 (m, 1H), |
| | | 1.92 (d, J = 10.0 Hz, 2H), 1.68-1.57 (m, 2H) |
| I-527 | 901.5 | 11.10 (s, 1H), 7.74-7.62 (m, 1H), 7.46 (s, 1H), 7.43-7.35 (m, 1H), 7.11-7.04 |
| | | (m, 1H), 6.99 (d, J = 5.6 Hz, 2H), 6.96-6.92 (m, 1H), 6.87-6.79 (m, 1H), 6.66 |
| | | (s, 1H), 6.60 (d, J = 8.0 Hz, 1H), 6.24-6.12 (m, 1H), 5.42-5.35 (m, 1H), 4.61- |
| | | 4.44 (m, 1H), 4.36-4.29 (m, 2H), 4.03-3.87 (m, 1H), 3.71-3.67 (m, 3H), |
| | | 3.62 (s, 3H), 3.29 (s, 4H), 3.26-3.18 (m, 4H), 3.17-3.11 (m, 2H), 2.98 (d, J = |
| | | 1.2 Hz, 6H), 2.95-2.81 (m, 4H), 2.78-2.75 (m, 1H), 2.73 (d, J = 4.4 Hz, 1H), |
| | | 2.67 (d, J = 4.0 Hz, 6H), 2.59 (d, J = 11.2 Hz, 1H), 2.06-1.96 (m, 2H), 1.96- |
| | | 1.83 (m, 1H), 1.79-1.71 (m, 1H), 1.55-1.38 (m, 1H) |
| I-529 | 871.3 | 11.09 (s, 1H), 7.69 (dd, J = 2.0, 10.8 Hz, 1H), 7.49 (d, J = 1.6 Hz, 1H), 7.40 |
| | | (dd, J = 1.2, 16.4 Hz, 1H), 7.29 (dd, J = 6.4, 8.4 Hz, 2H), 7.04 (dd, J = 3.2, 8.8 |
| | | Hz, 2H), 6.99 (d, J = 5.6 Hz, 2H), 6.96-6.92 (m, 2H), 6.18 (td, J = 2.0, 8.4 Hz, |
| | | 1H), 5.37 (dd, J = 5.6, 12.4 Hz, 1H), 4.60-4.45 (m, 1H), 4.37-4.30 (m, 2H), |
| | | 4.05-3.87 (m, 1H), 3.62 (s, 3H), 3.24 (d, J = 8.8 Hz, 6H), 3.15-3.10 (m, 2H), |
| | | 3.06 (d, J = 12.4 Hz, 1H), 2.99 (d, J = 1.2 Hz, 6H), 2.93-2.90 (m, 2H), 2.89- |
| | | 2.84 (m, 1H), 2.78 (d, J = 9.6 Hz, 1H), 2.76-2.71 (m, 1H), 2.70-2.62 (m, 8H), |
| | | 2.59 (s, 1H), 2.08-1.98 (m, 2H), 1.96-1.85 (m, 1H), 1.78-1.71 (m, 1H), |
| | | 1.50-1.35 (m, 1H) |
| I-530 | 859.7 | 11.81-11.75 (m, 1H), 11.10 (br s, 1H), 7.43-7.39 (m, 1H), 7.36-7.30 (m, |
| | | 1H), 7.09 (br d, J = 9.2 Hz, 1H), 7.00-6.93 (m, 4H), 6.47-6.41 (m, 1H), 6.02 |
| | | (s, 1H), 5.38 (dd, J = 5.2, 12.8 Hz, 1H), 4.31-4.21 (m, 2H), 3.63-3.57 (m, |
| | | 5H), 3.20-3.08 (m, 6H), 2.95-2.81 (m, 2H), 2.70-2.59 (m, 9H), 2.54 (s, 2H), |
| | | 2.39-2.32 (m, 2H), 2.29-2.23 (m, 1H), 2.07 (s, 2H), 2.04-1.99 (m, 2H), 1.23 |
| | | (s, 1H) |
| I-532 | 985.4 | 11.09 (s, 1H), 8.10 (d, J = 10.8 Hz, 1H), 7.68 (d, J = 10.0 Hz, 1H), 7.58 (s, 1H), |
| | | 7.53-7.44 (m, 1H), 7.43-7.27 (m, 1H), 7.16-7.06 (m, 1H), 7.01 (dd, J = 4.4, |
| | | 8.4 Hz, 1H), 6.97 (s, 1H), 6.93-6.86 (m, 1H), 6.21-6.04 (m, 1H), 5.37 (dd, |
| | | J = 5.2, 12.4 Hz, 1H), 4.62 (q, J = 7.2 Hz, 2H), 4.32 (d, J = 14.0 Hz, 2H), 3.68- |
| | | 3.63 (m, 1H), 3.62 (s, 3H), 3.60-3.57 (m, 1H), 3.51-3.38 (m, 2H), 3.18-3.04 |
| | | (m, 6H), 3.00 (s, 3H), 2.93-2.70 (m, 2H), 2.58 (s, 8H), 2.38-2.24 (m, 2H), |
| | | 2.08-1.95 (m, 1H), 0.71 (d, J = 6.4 Hz, 2H), 0.58 (s, 2H) |
| I-533 | 888.1 | 10.86 (s, 1H), 7.71 (dd, J = 1.8, 11.6 Hz, 1H), 7.57-7.49 (m, 2H), 7.45-7.38 |
| | | (m, 1H), 7.38-7.29 (m, 2H), 7.13-7.04 (m, 2H), 7.04-6.96 (m, 2H), 6.22- |
| | | 6.16 (m, 1H), 6.16-6.10 (m, 1H), 4.39-4.32 (m, 3H), 4.27 (s, 1H), 3.88 (dd, |
| | | J = 5.2, 12.0 Hz, 1H), 3.67-3.55 (m, 2H), 3.30 (s, 4H), 3.19 (s, 3H), 3.04- |
| | | 2.92 (m, 5H), 2.86-2.77 (m, 2H), 2.73-2.57 (m, 7H), 2.41-2.17 (m, 4H), |
| | | 2.06-1.96 (m, 1H) |
| I-534 | 479.7^{b} | 11.12 (s, 1H), 7.72 (dd, J = 2.0, 11.6 Hz, 1H), 7.57-7.50 (m, 2H), 7.45-7.31 |
| | | (m, 2H), 7.11 (d, J = 8.8 Hz, 1H), 7.05-6.97 (m, 2H), 6.95-6.88 (m, 1H), 6.19 |
| | | (td, J = 2.0, 13.2 Hz, 1H), 6.13 (d, J = 10.4 Hz, 1H), 5.39 (dd, J = 5.2, 12.8 Hz, |
| | | 1H), 4.40-4.33 (m, 3H), 4.27 (s, 1H), 3.67-3.56 (m, 5H), 3.24-3.07 (m, 6H), |
| | | 3.07-2.79 (m, 7H), 2.78-2.56 (m, 9H), 2.41-2.22 (m, 3H), 2.05-1.97 (m, |
| | | 1H) |
| I-537 | 739.4 | 12.30-12.07 (m, 1H), 11.07-10.70 (m, 1H), 7.38-7.29 (m, 2H), 7.25-7.18 |
| | | (m, 2H), 7.13 (s, 1H), 7.09-6.96 (m, 3H), 6.83 (s, 1H), 6.16 (s, 1H), 4.35 (d, |
| | | J = 8.4 Hz, 2H), 4.16 (dd, J = 5.2, 12.4 Hz, 1H), 3.62 (td, J = 5.6, 11.6 Hz, 2H), |
| | | 3.21 (s, 6H), 3.10-2.96 (m, 3H), 2.80-2.71 (m, 3H), 2.62-2.53 (m, 7H), 2.41- |
| | | 2.24 (m, 4H), 2.08 (d, J = 7.6 Hz, 3H), 2.02-1.94 (m, 1H) |
| I-539 | 755.5 | 12.16-11.99 (m, 1H), 10.91 (s, 1H), 7.43 (s, 1H), 7.31 (s, 2H), 7.15-7.08 (m, |
| | | 1H), 7.04-6.89 (m, 3H), 6.46 (s, 1H), 6.17-6.08 (m, 1H), 4.37-4.31 (m, 2H), |
| | | 4.20 (dd, J = 5.2, 12.4 Hz, 1H), 3.64-3.57 (m, 2H), 3.53 (s, 2H), 3.48-3.42 |
| | | (m, 3H), 3.20-2.95 (m, 10H), 2.82-2.73 (m, 1H), 2.63-2.52 (m, 5H), 2.39- |
| | | 2.29 (m, 2H), 2.28-2.24 (m, 1H), 2.07 (d, J = 6.8 Hz, 3H), 2.03-1.96 (m, 1H) |
| I-560 | 870.8 | 11.97-11.90 (m, 1H), 10.53-10.47 (m, 1H), 8.13-8.09 (m, 1H), 7.71-7.60 |
| | | (m, 2H), 7.44 (d, J = 10.0 Hz, 1H), 7.17 (d, J = 8.4 Hz, 1H), 6.90-6.81 (m, |
| | | 1H), 6.66 (s, 1H), 6.59 (dd, J = 1.8, 8.4 Hz, 1H), 6.49-6.46 (m, 1H), 6.14- |
| | | 6.06 (m, 1H), 4.65-4.59 (m, 2H), 4.38-4.29 (m, 2H), 3.72-3.70 (m, 3H), |
| | | 3.67-3.63 (m, 1H), 3.62-3.57 (m, 2H), 3.24 (br s, 6H), 3.15-3.02 (m, 6H), |
| | | 2.89-2.82 (m, 2H), 2.76-2.71 (m, 2H), 2.65-2.63 (m, 2H), 2.57 (br d, J = |
| | | 2.4 Hz, 4H), 2.54 (s, 1H), 2.36-2.26 (m, 2H) |
| I-561 | 844.6 | 12.18-12.09 (m, 1H), 10.62-10.42 (m, 1H), 7.52-7.42 (m, 2H), 7.28-7.21 |
| | | (m, 1H), 7.10-7.02 (m, 2H), 6.98-6.87 (m, 2H), 6.56-6.49 (m, 1H), 6.17- |
| | | 6.07 (m, 1H), 4.35-4.30 (m, 2H), 4.24 (s, 3H), 3.88 (t, J = 6.4 Hz, 2H), 3.64- |
| | | 3.57 (m, 2H), 3.27 (br s, 4H), 3.22-3.01 (m, 7H), 2.77 (t, J = 6.8 Hz, 2H), 2.73- |
| | | 2.67 (m, 6H), 2.37 (br d, J = 2.4 Hz, 2H), 2.29-2.23 (m, 1H), 2.07 (d, J = 9.2 |
| | | Hz, 3H) |
| I-585 | 830.2 | 12.14 (s, 1H), 10.66 (s, 1H), 8.17 (d, J = 6.4 Hz, 1H), 7.55 (s, 1H), 7.46-7.42 |
| | | (m, 1H), 7.20 (d, J = 6.4 Hz, 1H), 7.07 (d, J = 8.8 Hz, 1H), 6.96 (s, 1H), 6.94- |
| | | 6.88 (m, 2H), 6.53 (d, J = 2.4 Hz, 1H), 6.14-6.09 (m, 1H), 4.34-4.30 (m, 2H), |
| | | 3.80 (t, J = 6.4 Hz, 2H), 3.64-3.58 (m, 2H), 3.28 (s, 4H), 3.16 (s, 1H), 3.13 (s, |
| | | 3H), 3.08-2.97 (m, 3H), 2.85-2.77 (m, 4H), 2.66 (s, 4H), 2.37 (s, 2H), 2.30- |
| | | 2.23 (m, 1H), 2.08 (s, 2H), 2.06 (s, 1H) |
| I-588 | 804.3 | 12.27-12.12 (m, 1H), 10.77 (s, 1H), 7.72 (dd, J = 1.6, 11.1 Hz, 1H), 7.64 (d, |
| | | J = 7.6 Hz, 2H), 7.45 (d, J = 8.0 Hz, 3H), 7.11-6.98 (m, 1H), 6.90-6.79 (m, |
| | | 2H), 6.51 (d, J = 15.2 Hz, 1H), 6.42 (d, J = 8.8 Hz, 1H), 6.23-6.12 (m, 2H), |
| | | 5.80 (d, J = 7.2 Hz, 1H), 4.44-4.33 (m, 3H), 4.30-4.22 (m, 1H), 3.70-3.53 |
| | | (m, 4H), 3.24-3.05 (m, 6H), 3.05-2.95 (m, 3H), 2.89 (s, 4H), 2.79-2.61 (m, |
| | | 2H), 2.60-2.53 (m, 4H), 2.37-2.25 (m, 2H), 2.08 (td, J = 4.4, 8.8 Hz, 1H), |
| | | 1.91-1.75 (m, 1H) |
| I-616 | 767.8 | 12.08-12.03 (m, 1H), 10.85 (s, 1H), 7.32 (br t, J = 8.0 Hz, 1H), 7.20 (br d, |
| | | J = 8.4 Hz, 1H), 7.08-7.03 (m, 1H), 7.02-7.00 (m, 1H), 6.97 (br d, J = 6.0 Hz, |
| | | 1H), 6.90 (br d, J = 8.4 Hz, 1H), 6.57-6.52 (m, 1H), 6.13 (br s, 1H), 4.33 (br |
| | | d, J = 6.8 Hz, 2H), 3.91-3.83 (m, 1H), 3.63-3.57 (m, 2H), 3.24-3.19 (m, |
| | | 4H), 3.16-2.99 (m, 6H), 2.92 (br s, 4H), 2.83-2.78 (m, 2H), 2.70-2.56 (m, |
| | | 8H), 2.40-2.30 (m, 2H), 2.23 (s, 4H), 2.07 (br d, J = 7.2 Hz, 3H) |
| I-669 | 850.2 | 10.88-10.80 (m, 1H), 7.72 (d, J = 2.0 Hz, 1H), 7.43-7.28 (m, 3H), 7.26-7.15 |
| | | (m, 2H), 7.07-6.99 (m, 2H), 6.69-6.60 (m, 2H), 6.24-6.15 (m, 2H), 4.39- |
| | | 4.30 (m, 4H), 3.90-3.84 (m, 1H), 3.70 (d, J = 2.8 Hz, 3H), 3.65-3.56 (m, 2H), |
| | | 3.26 (s, 4H), 2.98 (s, 8H), 2.84-2.79 (m, 2H), 2.72-2.66 (m, 1H), 2.65-2.56 |
| | | (m, 7H), 2.34-2.17 (m, 3H), 2.06-1.99 (m, 1H) |
| I-814 | 859.4 | 12.18 (br s, 1H), 11.10 (br s, 1H), 8.27 (br d, J = 19.2 Hz, 1H), 7.45-7.39 (m, |
| | | 1H), 7.25-7.18 (m, 2H), 7.00-6.93 (m, 4H), 6.53-6.48 (m, 1H), 6.12 (br s, |
| | | 1H), 5.41-5.34 (m, 1H), 4.34 (br s, 2H), 3.61 (s, 6H), 3.14 (br s, 8H), 3.07- |
| | | 2.99 (m, 3H), 2.96-2.82 (m, 2H), 2.72 (br s, 2H), 2.67 (br s, 4H), 2.41-2.32 |
| | | (m, 2H), 2.31-2.23 (m, 1H), 2.10-2.06 (m, 3H), 2.04-1.99 (m, 1H) |
| I-820 | 811.3 | 12.20 (br s, 1H), 10.89 (s, 1H), 7.57 (d, J = 8.4 Hz, 1H), 7.27-7.20 (m, 2H), |
| | | 7.15-7.09 (m, 1H), 7.07-7.02 (m, 1H), 6.99-6.97 (m, 1H), 6.92-6.89 (m, |
| | | 1H), 6.49 (br s, 1H), 6.18-6.13 (m, 1H), 4.38-4.32 (m, 3H), 4.25 (s, 3H), 3.64- |
| | | 3.57 (m, 2H), 3.27 (br d, J = 8.8 Hz, 2H), 3.16 (br s, 6H), 3.09-2.99 (m, 3H), |
| | | 2.73 (br s, 6H), 2.68-2.62 (m, 2H), 2.45-2.30 (m, 3H), 2.29-2.22 (m, 1H), |
| | | 2.21-2.14 (m, 1H), 2.07 (d, J = 6.0 Hz, 3H) |
| I-821 | 839.1 | 12.24-12.11 (m, 1H), 10.89 (s, 1H), 7.56 (d, J = 8.4 Hz, 1H), 7.31 (br d, J = |
| | | 13.2 Hz, 1H), 7.20 (br dd, J = 7.6, 15.2 Hz, 2H), 7.04 (t, J = 7.6 Hz, 1H), 6.99- |
| | | 6.89 (m, 1H), 6.56-6.46 (m, 1H), 6.19-6.09 (m, 1H), 4.39-4.35 (m, 2H), |
| | | 4.25 (s, 3H), 3.68-3.63 (m, 2H), 3.27 (br s, 4H), 3.16 (br s, 6H), 3.07-3.00 |
| | | (m, 2H), 2.98-2.93 (m, 1H), 2.76-2.62 (m, 8H), 2.36 (br s, 2H), 2.34-2.28 |
| | | (m, 1H), 2.28-2.20 (m, 1H), 2.17 (br dd, J = 5.2, 13.2 Hz, 1H), 1.07-0.97 (m, |
| | | 6H) |
| I-822 | 823.2 | 12.23-12.17 (m, 1H), 10.90 (br s, 1H), 7.57 (br d, J = 7.6 Hz, 1H), 7.42-7.35 |
| | | (m, 1H), 7.23 (br d, J = 6.4 Hz, 1H), 7.08-6.99 (m, 3H), 6.65 (br s, 1H), 6.19- |
| | | 6.11 (m, 1H), 4.40-4.36 (m, 2H), 4.25 (s, 3H), 3.67 (br d, J = 4.8 Hz, 2H), |
| | | 3.17 (br s, 8H), 3.07-3.04 (m, 1H), 2.98-2.94 (m, 1H), 2.72 (br s, 6H), 2.70- |
| | | 2.62 (m, 4H), 2.58-2.56 (m, 1H), 2.41-2.36 (m, 2H), 2.34 (br d, J = 4.0 Hz, |
| | | 1H), 2.28-2.14 (m, 2H), 1.07-1.00 (m, 6H) |
| I-832 | 788.4 | 12.00 (d, J = 2.0 Hz, 1H), 11.06 (s, 1H), 7.39 (d, J = 8.4 Hz, 1H), 7.32-7.23 |
| | | (m, 2H), 7.04 (s, 1H), 6.94 (t, J = 6.4 Hz, 3H), 6.48 (s, 1H), 6.23-5.99 (m, |
| | | 1H), 5.79-5.64 (m, 1H), 4.32 (d, J = 11.2 Hz, 2H), 3.74 (s, 3H), 3.64- 3.57 |
| | | (m, 2H), 3.21-3.12 (m, 8H), 3.10-2.99 (m, 3H), 2.88-2.80 (m, 1H), 2.73 (d, |
| | | J = 2.4 Hz, 1H), 2.70 (s, 1H), 2.66 (s, 3H), 2.63 (d, J = 7.6 Hz, 2H), 2.37 (s, |
| | | 1H), 2.28-2.14 (m, 4H), 2.07 (d, J = 7.2 Hz, 3H), 1.89-1.75 (m, 4H) |
| I-834 | 823.5 | 12.20 (s, 1H), 11.06 (s, 1H), 7.45-7.22 (m, 3H), 7.12-6.89 (m, 3H), 6.65 (s, |
| | | 1H), 6.16 (s, 1H), 5.73 (dd, J = 5.2, 11.6 Hz, 1H), 4.45-4.31 (m, 2H), 3.67 (d, |
| | | J = 5.2 Hz, 2H), 3.26-3.02 (m, 12H), 2.96 (dd, J = 6.4, 13.2 Hz, 1H), 2.89- |
| | | 2.81 (m, 1H), 2.77-2.64 (m, 11H), 2.38 (s, 1H), 2.32-2.17 (m, 2H), 1.08- |
| | | 0.99 (m, 6H) |
| I-845 | 807.3 | 12.11 (s, 1H), 10.90 (s, 1H), 7.57 (d, J = 7.6 Hz, 1H), 7.23 (d, J = 6.8 Hz, 1H), |
| | | 7.14 (d, J = 8.4 Hz, 1H), 7.08-7.02 (m, 1H), 7.01-6.91 (m, 1H), 6.87 (t, J = |
| | | 8.0 Hz, 1H), 6.58 (s, 1H), 6.15 (s, 1H), 4.40-4.33 (m, 3H), 4.26 (s, 3H), 3.65- |
| | | 3.54 (m, 6H), 3.14 (s, 8H), 3.07-2.99 (m, 2H), 2.77-2.61 (m, 9H), 2.41- |
| | | 2.35 (m, 2H), 2.27 (d, J = 1.2 Hz, 1H), 2.21-2.15 (m, 1H), 2.08 (d, J = 6.0 Hz, |
| | | 3H) |
| I-855 | 816.3 | 12.12 (s, 1H), 11.09 (s, 1H), 7.23 (s, 1H), 7.21 (s, 1H), 7.16 (d, J = 8.4 Hz, 1H), |
| | | 6.98 (d, J = 4.0 Hz, 2H), 6.93 (d, J = 4.4 Hz, 1H), 6.88-6.79 (m, 1H), 6.38 (d, |
| | | J = 0.8 Hz, 1H), 6.14 (s, 1H), 5.41-5.34 (m, 1H), 4.41-4.34 (m, 2H), 3.69- |
| | | 3.63 (m, 2H), 3.61 (s, 3H), 3.13 (d, J = 1.2 Hz, 3H), 3.10 (d, J = 6.4 Hz, 6H), |
| | | 2.94 (s, 2H), 2.72 (d, J = 2.8 Hz, 1H), 2.68-2.63 (m, 2H), 2.63-2.57 (m, 3H), |
| | | 2.38-2.32 (m, 2H), 2.25-2.19 (m, 1H), 2.14 (s, 3H), 2.03-1.97 (m, 1H), 1.88- |
| | | 1.80 (m, 2H), 1.80-1.70 (m, 2H), 1.24 (s, 1H), 1.04 (d, J = 6.8 Hz, 3H), 1.00 |
| | | (d, J = 5.6 Hz, 3H) |
| I-886 | 859.0 | 12.27 (s, 1H), 11.08 (s, 1H), 8.04 (d, J = 8.0 Hz, 1H), 7.44 (dd, J = 3.2, 8.0 Hz, |
| | | 1H), 7.11-6.91 (m, 4H), 6.56 (s, 1H), 6.22-6.10 (m, 1H), 5.37 (dd, J = 5.6, |
| | | 12.8 Hz, 1H), 4.34 (d, J = 9.6 Hz, 2H), 3.64-3.57 (m, 5H), 3.19-3.02 (m, 9H), |
| | | 2.94-2.85 (m, 1H), 2.74 (dd, J = 4.0, 12.4 Hz, 2H), 2.69-2.59 (m, 4H), 2.38 |
| | | (s, 1H), 2.27 (d, J = 2.0 Hz, 1H), 2.18 (t, J = 10.8 Hz, 2H), 2.07 (d, J = 8.8 Hz, |
| | | 3H), 2.04-1.97 (m, 1H), 1.96-1.89 (m, 2H), 1.86-1.76 (m, 2H) |
| I-892 | 869.7 | 12.12 (d, J = 6.0 Hz, 1H), 11.10 (s, 1H), 7.70-7.63 (m, 1H), 7.57-7.49 (m, |
| | | 2H), 7.47-7.34 (m, 1H), 7.12-7.03 (m, 2H), 7.01-6.91 (m, 4H), 6.86 (s, 1H), |
| | | 6.23-6.16 (m, 1H), 6.12 (s, 1H), 5.44-5.32 (m, 1H), 4.41-4.19 (m, 3H), 4.14- |
| | | 4.06 (m, 1H), 3.73-3.64 (m, 1H), 3.62 (s, 3H), 3.58-3.43 (m, 3H), 3.25 (s, |
| | | 6H), 3.17-3.09 (m, 4H), 2.88 (dd, J = 5.2, 16.4 Hz, 1H), 2.73 (d, J = 4.0 Hz, |
| | | 1H), 2.71-2.59 (m, 8H), 2.35-2.21 (m, 2H), 2.05-1.94 (m, 1H), 1.06-0.96 |
| | | (m, 3H) |
| I-894 | 869.8 | 0.95-1.05 (m, 3 H) 1.94-2.06 (m, 1 H) 2.15-2.32 (m, 2 H) 2.57-2.76 (m, 9 |
| | | H) 2.84-2.94 (m, 1 H) 3.06-3.16 (m, 5 H) 3.25 (br s, 5 H) 3.41-3.74 (m, 7 |
| | | H) 4.04-4.14 (m, 1 H) 4.25-4.42 (m, 3 H) 5.37 (dd, J = 12.8, 5.6 Hz, 1 H) |
| | | 6.07-6.23 (m, 2 H) 6.86 (d, J = 2.4 Hz, 1 H) 6.90-7.01 (m, 4 H) 7.03-7.11 |
| | | (m, 2 H) 7.33-7.47 (m, 1 H) 7.49-7.57 (m, 2 H) 7.62-7.69 (m, 1 H) 11.09 |
| | | (br s, 1 H) 12.11 (br d, J = 4.8 Hz, 1 H) |
| I-896 | 881.6 | 12.12 (s, 1H), 11.10 (s, 1H), 7.71 (s, 1H), 7.54 (d, J = 8.8 Hz, 2H), 7.40 (br s, |
| | | 1H), 7.08 (br d, J = 8.8 Hz, 2H), 7.02-6.91 (m, 4H), 6.87 (br s, 1H), 6.28- |
| | | 6.07 (m, 2H), 5.38 (br dd, J = 5.2, 12.8 Hz, 1H), 4.29 (s, 4H), 3.62 (s, 3H), 3.54 |
| | | (br s, 2H), 3.25 (br s, 5H), 3.20-3.00 (m, 7H), 2.89 (br dd, J = 5.2, 16.8 Hz, |
| | | 1H), 2.74-2.62 (m, 8H), 2.28 (br s, 2H), 2.06-1.97 (m, 1H), 0.92 (br s, 4H) |
| I-914 | 855.0 | 12.02 (s, 1H), 11.04 (s, 1H), 7.44-7.30 (m, 1H), 7.00-6.97 (m, 1H), 7.00- |
| | | 6.93 (m, 3H), 6.88 (d, J = 5.6 Hz, 1H), 6.57-6.52 (m, 1H), 6.18-6.05 (m, 1H), |
| | | 5.44-5.31 (m, 1H), 4.35-4.30 (m, 2H), 3.65-3.62 (m, 1H), 3.62-3.60 (m, |
| | | 3H), 3.60-3.57 (m, 1H), 3.25 (s, 6H), 3.15-3.10 (m, 4H), 3.07-3.04 (m, 1H), |
| | | 3.02-2.84 (m, 2H), 2.77-2.68 (m, 2H), 2.67 (d, J = 3.6 Hz, 4H), 2.48-2.41 |
| | | (m, 1H), 2.37 (s, 4H), 2.09-2.05 (m, 3H), 2.04-1.95 (m, 1H), 1.61-1.52 (m, |
| | | 3H) |
| I-929 | 827.0 | 1.98-2.10 (m, 4 H) 2.22-2.29 (m, 1 H) 2.31-2.40 (m, 2 H) 2.67 (br dd, J = |
| | | 16.0, 1.6 Hz, 7 H) 3.15 (br s, 12 H) 3.57-3.63 (m, 6 H) 4.33 (br d, J = 9.2 Hz, |
| | | 2 H) 5.32-5.42 (m, 1 H) 6.11-6.19 (m, 1 H) 6.50 (br s, 1 H) 6.88-7.00 (m, 4 |
| | | H) 7.18 (br d, J = 8.0 Hz, 1 H) 7.31 (br d, J = 14.0 Hz, 1 H) 11.10 (br s, 1 H) |
| | | 12.20 (br s, 1 H) |
| I-964 | 807.6 | 0.98-1.06 (m, 6 H) 1.98-2.07 (m, 1 H) 2.20-2.33 (m, 1 H) 2.20-2.37 (m, 1 |
| | | H) 2.54-2.79 (m, 8 H) 2.83-3.01 (m, 3 H) 3.02-3.23 (m, 7 H) 3.64-3.69 |
| | | (m, 2 H) 3.70-3.77 (m, 5 H) 4.32-4.40 (m, 2 H) 5.34-5.44 (m, 1 H) 6.12 (br |
| | | s, 1 H) 6.60 (br s, 1 H) 6.86 (br d, J = 12 Hz, 2 H) 6.89-6.96 (m, 2 H) 6.97- |
| | | 7.03 (m, 1 H) 7.10 (br d, J = 7.6 Hz, 1 H) 7.39 (t, J = 8.8 Hz, 1 H) 11.10 (br s, |
| | | 1 H) 12.12 (br s, 1 H) |
| I-966 | 891.4 | 12.15-12.10 (m, 1H), 10.89 (s, 1H), 7.56 (d, J = 8.4 Hz, 1H), 7.40 (t, J = 8.8 |
| | | Hz, 1H), 7.22 (d, J = 6.8 Hz, 1H), 7.08-7.02 (m, 1H), 6.97-6.87 (m, 3H), 6.61 |
| | | (br s, 1H), 6.13 (br s, 1H), 4.36 (br dd, J = 5.2, 9.6 Hz, 3H), 4.25 (s, 3H), 3.69- |
| | | 3.59 (m, 4H), 3.27-3.21 (m, 3H), 3.16-3.02 (m, 6H), 3.01-2.91 (m, 3H), |
| | | 2.67-2.61 (m, 4H), 2.60-2.55 (m, 2H), 2.39-2.34 (m, 2H), 2.30-2.23 (m, |
| | | 1H), 2.21-2.14 (m, 1H), 1.12 (br d, J = 5.2 Hz, 3H), 1.06-0.99 (m, 6H) |
| I-971 | 824.4 | 11.94 (s, 1H), 11.06 (s, 1H), 7.40 (d, J = 8.4 Hz, 1H), 7.36-7.26 (m, 2H), 7.12- |
| | | 7.06 (m, 1H), 7.05-6.89 (m, 3H), 6.49 (d, J = 2.4 Hz, 1H), 6.19-6.09 (m, |
| | | 1H), 5.73 (d, J = 5.2 Hz, 1H), 4.37-4.30 (m, 2H), 3.74 (s, 3H), 3.61 (d, J = 5.6 |
| | | Hz, 2H), 3.24-3.14 (m, 6H), 3.10-2.95 (m, 3H), 2.88-2.82 (m, 1H), 2.80- |
| | | 2.73 (m, 3H), 2.70 (s, 1H), 2.67 (s, 3H), 2.60 (d, J = 11.8 Hz, 1H), 2.54 (s, 3H), |
| | | 2.39-2.33 (m, 2H), 2.27-2.20 (m, 2H), 2.08 (d, J = 6.4 Hz, 3H), 1.96-1.88 |
| | | (m, 1H) |
| I-972 | 824.4 | 12.02 (s, 1H), 11.05 (s, 1H), 7.40 (d, J = 8.4 Hz, 1H), 7.35-7.26 (m, 2H), 7.09 |
| | | (s, 1H), 7.05-6.89 (m, 3H), 6.49 (s, 1H), 6.20-6.09 (m, 1H), 5.73 (d, J = 5.2 |
| | | Hz, 1H), 4.39-4.27 (m, 2H), 3.74 (s, 3H), 3.61 (d, J = 5.6 Hz, 2H), 3.24-3.14 |
| | | (m, 6H), 3.11-2.99 (m, 3H), 2.88-2.81 (m, 1H), 2.80-2.73 (m, 3H), 2.72- |
| | | 2.69 (m, 1H), 2.67 (s, 3H), 2.62-2.55 (m, 1H), 2.54 (s, 3H), 2.39-2.33 (m, |
| | | 2H), 2.27-2.20 (m, 2H), 2.08 (d, J = 6.4 Hz, 3H), 1.97-1.86 (m, 1H) |
| I-983 | 873.5 | δ (ppm) = 12.14 (br s, 1H), 11.11 (br s, 1H), 7.43 (br d, J = 8.8 Hz, 1H), 7.11 |
| | | (br d, J = 7.6 Hz, 1H), 7.05 (br d, J = 8.0 Hz, 1H), 7.02-6.97 (m, 1H), 6.94 (br |
| | | d, J = 6.4 Hz, 2H), 6.89 (br d, J = 6.0 Hz, 1H), 6.53 (br s, 1H), 6.10 (br s, 1H), |
| | | 5.45-5.35 (m, 1H), 4.39-4.31 (m, 2H), 3.75 (s, 2H), 3.72 (s, 3H), 3.68-3.63 |
| | | (m, 2H), 3.25 (br s, 4H), 3.20-3.00 (m, 6H), 2.96-2.86 (m, 2H), 2.78-2.65 |
| | | (m, 2H), 2.59 (br s, 4H), 2.38-2.34 (m, 1H), 2.25 (br s, 1H), 2.06-1.98 (m, |
| | | 1H), 1.04 (br d, J = 6.8 Hz, 3H), 0.99 (br d, J = 6.8 Hz, 3H) |
| I-990 | 810.4 | 11.92 (br d, J = 5.4 Hz, 1H), 10.88-10.79 (m, 1H), 8.20-7.90 (m, 1H), 7.81- |
| | | 7.59 (m, 1H), 7.29 (t, J = 8.0 Hz, 1H), 7.06-6.98 (m, 3H), 6.80-6.71 (m, 1H), |
| | | 6.04 (br s, 1H), 4.62 (q, J = 6.4 Hz, 2H), 4.34-4.26 (m, 2H), 3.86 (br dd, J = |
| | | 4.4, 11.8 Hz, 1H), 3.66-3.57 (m, 2H), 3.32-3.31 (m, 6H), 3.11-3.05 (m, 4H), |
| | | 2.96 (br d, J = 3.4 Hz, 2H), 2.79-2.74 (m, 2H), 2.64 (br dd, J = 5.2, 12.0 Hz, |
| | | 2H), 2.56-2.52 (m, 4H), 2.33 (br s, 2H), 2.29-2.13 (m, 4H), 2.11-1.95 (m, |
| | | 6H), 1.53 (br s, 4H) |
| I-991 | 810.1 | 11.94-11.89 (m, 1H), 10.85-10.82 (m, 1H), 8.12 (s, 1H), 7.70 (s, 1H), 7.30- |
| | | 7.25 (m, 1H), 7.06-7.01 (m, 1H), 7.00-6.97 (m, 1H), 6.95-6.92 (m, 1H), |
| | | 6.79-6.73 (m, 1H), 6.07-6.02 (m, 1H), 4.63 (s, 2H), 4.33-4.26 (m, 2H), |
| | | 3.89-3.83 (m, 1H), 3.58 (t, J = 5.6 Hz, 2H), 3.25-3.16 (m, 3H), 3.09 (d, J = |
| | | 6.4 Hz, 5H), 2.86 (s, 1H), 2.76-2.61 (m, 4H), 2.55 (s, 4H), 2.48-2.45 (m, |
| | | 4H), 2.40-2.38 (m, 1H), 2.33 (s, 4H), 2.04-1.99 (m, 1H), 1.95-1.89 (m, |
| | | 4H), 1.64-1.38 (m, 5H) |
| I-1080 | 781.1 | 11.95-11.89 (m, 1H), 11.09 (s, 1H), 6.99-6.94 (m, 3H), 6.93-6.89 (m, 1H), |
| | | 6.82-6.74 (m, 1H), 6.06 (br s, 1H), 5.41-5.33 (m, 1H), 4.34-4.25 (m, 2H), |
| | | 3.65-3.61 (m, 1H), 3.59 (s, 3H), 3.27-2.96 (m, 9H), 2.94-2.84 (m, 2H), |
| | | 2.79-2.63 (m, 3H), 2.63-2.53 (m, 8H), 2.40-2.33 (m, 2H), 2.29-2.22 (m, |
| | | 1H), 2.10-2.05 (m, 3H), 2.04-1.87 (m, 6H), 1.70-1.55 (m, 2H), 1.53-1.42 |
| | | (m, 2H) |
| I-1081 | 805.3 | 12.19 (s, 1H), 11.05 (s, 1H), 7.47-7.36 (m, 3H), 7.28 (t, J = 7.6 Hz, 1H), |
| | | 7.15 (t, J = 8.8 Hz, 1H), 7.07-6.99 (m, 1H), 6.96 (d, J = 7.0 Hz, 1H), 6.88 (s, |
| | | 1H), 6.17 (s, 1H), 5.72 (dd, J = 4.8, 12.0 Hz, 1H), 4.44-4.34 (m, 2H), 3.67 |
| | | (d, J = 5.6 Hz, 2H), 3.39-3.33 (m, 2H), 3.29-3.28 (m, 1H), 3.21-3.17 (m, |
| | | 3H), 3.14 (s, 5H), 3.09-3.02 (m, 2H), 2.99-2.93 (m, 1H), 2.88-2.80 (m, |
| | | 1H), 2.71 (d, J = 10.8 Hz, 5H), 2.66 (s, 5H), 2.37 (s, 1H), 2.33-2.18 (m, 2H), |
| | | 1.07-0.98 (m, 6H) |
| I-1082 | 805.6 | 12.21 (br s, 1H), 10.89 (s, 1H), 7.57 (d, J = 8.0 Hz, 1H), 7.45 (br d, J = 10.4 |
| | | Hz, 2H), 7.23 (d, J = 7.2 Hz, 1H), 7.16 (br t, J = 8.8 Hz, 1H), 7.08-6.99 (m, |
| | | 2H), 6.89 (t, J = 2.4 Hz, 1H), 6.18 (br s, 1H), 4.43-4.34 (m, 3H), 4.25 (s, |
| | | 3H), 3.67 (br d, J = 5.6 Hz, 2H), 3.27 (br d, J = 8.4 Hz, 3H), 3.14 (br s, 9H), |
| | | 3.01-2.93 (m, 1H), 2.77-2.59 (m, 8H), 2.38 (br d, J = 4.4 Hz, 1H), 2.36- |
| | | 2.31 (m, 1H), 2.27 (br s, 1H), 2.22-2.14 (m, 1H), 1.08-0.99 (m, 6H) |
| I-1083 | 982.2 | 12.10-11.97 (m, 1H), 11.13-11.03 (m, 1H), 7.83-7.74 (m, 1H), 7.32-7.15 |
| | | (m, 2H), 7.06-6.75 (m, 5H), 6.73-6.44 (m, 3H), 6.17-6.04 (m, 1H), 5.44- |
| | | 5.29 (m, 1H), 4.42-4.28 (m, 2H), 3.86-3.76 (m, 7H), 3.73 (s, 3H), 3.69- |
| | | 3.64 (m, 2H), 3.62 (s, 3H), 3.29-3.23 (m, 5H), 3.13 (br dd, J = 7.3, 8.5 Hz, |
| | | 2H), 2.99-2.76 (m, 3H), 2.70-2.59 (m, 10H), 2.37 (br d, J = 2.9 Hz, 1H), |
| | | 2.30-2.22 (m, 1H), 2.05-1.95 (m, 1H), 1.09-0.96 (m, 6H) |
| I-1084 | 800.5 | 13.56 (br d, J = 5.6 Hz, 1H), 10.91 (s, 1H), 8.28-7.99 (m, 1H), 7.88-7.78 |
| | | (m, 1H), 7.77-7.63 (m, 1H), 7.57 (d, J = 8.1 Hz, 1H), 7.28-7.19 (m, 2H), |
| | | 7.05 (t, J = 7.6 Hz, 1H), 6.99-6.87 (m, 1H), 6.70 (s, 1H), 6.63 (br d, J = 8.4 |
| | | Hz, 1H), 6.27-6.14 (m, 1H), 4.70-4.59 (m, 2H), 4.40-4.31 (m, 3H), 4.26 |
| | | (s, 3H), 3.79-3.72 (m, 3H), 3.69-3.59 (m, 2H), 3.32-3.27 (m, 6H), 3.15- |
| | | 3.06 (m, 2H), 2.76-2.65 (m, 8H), 2.39-2.27 (m, 3H), 2.22-2.13 (m, 1H) |
| I-1085 | 836.4 | 12.17-12.07 (m, 1H), 10.81-10.76 (m, 1H), 8.17-8.16 (m, 1H), 7.75-7.67 |
| | | (m, 1H), 7.45-7.32 (m, 2H), 7.03-6.86 (m, 4H), 6.62-6.57 (m, 1H), 6.48- |
| | | 6.43 (m, 2H), 6.22-6.16 (m, 1H), 6.15-6.09 (m, 1H), 6.03-5.98 (m, 1H), |
| | | 4.28 (br s, 5H), 3.67-3.62 (m, 1H), 3.60-3.53 (m, 2H), 3.26 (br d, J = 3.2 |
| | | Hz, 6H), 3.17-3.04 (m, 6H), 2.99-2.95 (m, 2H), 2.76-2.70 (m, 1H), 2.67- |
| | | 2.63 (m, 2H), 2.59 (br s, 4H), 2.34-2.31 (m, 1H), 2.29-2.22 (m, 1H), 2.13- |
| | | 2.07 (m, 1H), 1.92-1.84 (m, 1H) |
| I-1086 | 837.4 | 12.13 (d, J = 7.2 Hz, 1H), 10.79 (s, 1H), 8.14 (d, J = 12.8 Hz, 1H), 7.73- |
| | | 7.65 (m, 1H), 7.40 (t, J = 8.8 Hz, 1H), 7.06-6.91 (m, 2H), 6.88 (d, J = 10.8 |
| | | Hz, 2H), 6.64-6.57 (m, 1H), 6.52-6.42 (m, 2H), 6.18-6.09 (m, 1H), 6.01 |
| | | (d, J = 7.8 Hz, 1H), 4.68-4.58 (m, 2H), 4.40-4.28 (m, 3H), 3.68-3.58 (m, |
| | | 2H), 3.26 (s, 7H), 3.16-3.03 (m, 6H), 2.80-2.71 (m, 1H), 2.69-2.56 (m, |
| | | 7H), 2.45-2.24 (m, 3H), 2.13-2.06 (m, 1H), 1.93-1.83 (m, 1H) |
| I-1087 | 894.4 | 12.35-11.98 (m, 1H), 10.99-10.80 (m, 1H), 8.51-8.45 (m, 1H), 7.74-7.69 |
| | | (m, 1H), 7.60-7.48 (m, 4H), 7.46-7.38 (m, 1H), 7.28-7.21 (m, 1H), 7.20- |
| | | 7.13 (m, 1H), 7.11-6.99 (m, 1H), 6.93-6.87 (m, 1H), 6.26-6.17 (m, 1H), |
| | | 4.52-4.45 (m, 1H), 4.40-4.34 (m, 3H), 4.26 (s, 3H), 3.92-3.87 (m, 1H), |
| | | 3.56-3.42 (m, 4H), 3.23-3.05 (m, 8H), 2.95-2.89 (m, 2H), 2.82 (br t, J = |
| | | 8.0 Hz, 2H), 2.73-2.56 (m, 5H), 2.44-2.33 (m, 2H), 2.27-2.15 (m, 2H), |
| | | 1.98-1.87 (m, 3H), 1.77 (br d, J = 11.6 Hz, 1H), 1.59-1.42 (m, 1H) |
| I-1088 | 894.4 | 12.35-11.98 (m, 1H), 10.99-10.80 (m, 1H), 8.51-8.45 (m, 1H), 7.74-7.69 |
| | | (m, 1H), 7.60-7.48 (m, 4H), 7.46-7.38 (m, 1H), 7.28-7.21 (m, 1H), 7.20- |
| | | 7.13 (m, 1H), 7.11-6.99 (m, 1H), 6.93-6.87 (m, 1H), 6.26-6.17 (m, 1H), |
| | | 4.52-4.45 (m, 1H), 4.40-4.34 (m, 3H), 4.26 (s, 3H), 3.92-3.87 (m, 1H), |
| | | 3.56-3.42 (m, 4H), 3.23-3.05 (m, 8H), 2.95-2.89 (m, 2H), 2.82 (br t, J = |
| | | 8.0 Hz, 2H), 2.73-2.56 (m, 5H), 2.44-2.33 (m, 2H), 2.27-2.15 (m, 2H), |
| | | 1.98-1.87 (m, 3H), 1.77 (br d, J = 11.6 Hz, 1H), 1.59-1.42 (m, 1H) |
| I-1089 | 928.2 | 12.23-12.08 (m, 1H), 10.95-10.86 (m, 1H), 8.40-8.37 (m, 1H), 7.74-7.68 |
| | | (m, 1H), 7.67-7.63 (m, 1H), 7.58 (d, J = 8.0 Hz, 1H), 7.46-7.38 (m, 2H), |
| | | 7.25-7.20 (m, 1H), 7.08-7.04 (m, 2H), 6.51 (d, J = 2.4 Hz, 1H), 6.25-6.14 |
| | | (m, 1H), 4.52-4.45 (m, 1H), 4.37 (br t, J = 5.2 Hz, 3H), 4.27-4.24 (m, 3H), |
| | | 3.92-3.85 (m, 1H), 3.61-3.40 (m, 3H), 3.23-3.14 (m, 4H), 3.13-2.97 (m, |
| | | 6H), 2.95-2.88 (m, 2H), 2.82 (br t, J = 7.2 Hz, 2H), 2.72-2.59 (m, 4H), 2.44- |
| | | 2.33 (m, 2H), 2.31-2.23 (m, 1H), 2.21-2.15 (m, 1H), 1.97-1.86 (m, 3H), |
| | | 1.79-1.72 (m, 1H), 1.54-1.41 (m, 1H) |
| I-1090 | 926.5 | 12.32-12.19 (m, 1H), 11.12-11.01 (m, 1H), 7.75-7.60 (m, 2H), 7.47-7.36 |
| | | (m, 3H), 7.33-7.27 (m, 1H), 7.09-6.95 (m, 2H), 6.57-6.43 (m, 1H), 6.27- |
| | | 6.11 (m, 2H), 5.78-5.67 (m, 1H), 4.30 (br s, 4H), 3.69-3.60 (m, 1H), 3.58 |
| | | (br t, J = 5.6 Hz, 2H), 3.46-3.40 (m, 1H), 3.23-3.14 (m, 6H), 3.10-3.04 |
| | | (m, 2H), 3.02-2.95 (m, 3H), 2.89-2.73 (m, 5H), 2.67 (s, 3H), 2.36-2.21 |
| | | (m, 6H), 1.94-1.86 (m, 1H) |
| I-1091 | 928.2 | 12.23-12.08 (m, 1H), 10.95-10.86 (m, 1H), 8.40-8.37 (m, 1H), 7.74-7.68 |
| | | (m, 1H), 7.67-7.63 (m, 1H), 7.58 (d, J = 8.0 Hz, 1H), 7.46-7.38 (m, 2H), |
| | | 7.25-7.20 (m, 1H), 7.08-7.04 (m, 2H), 6.51 (d, J = 2.4 Hz, 1H), 6.25-6.14 |
| | | (m, 1H), 4.52-4.45 (m, 1H), 4.37 (br t, J = 5.2 Hz, 3H), 4.27-4.24 (m, 3H), |
| | | 3.92-3.85 (m, 1H), 3.61-3.40 (m, 3H), 3.23-3.14 (m, 4H), 3.13-2.97 (m, |
| | | 6H), 2.95-2.88 (m, 2H), 2.82 (br t, J = 7.2 Hz, 2H), 2.72-2.59 (m, 4H), 2.44- |
| | | 2.33 (m, 2H), 2.31-2.23 (m, 1H), 2.21-2.15 (m, 1H), 1.97-1.86 (m, 3H), |
| | | 1.79-1.72 (m, 1H), 1.54-1.41 (m, 1H) |
| I-1092 | 889.4 | 12.20 (d, J = 7.1 Hz, 1H), 10.56 (s, 1H), 9.56 (s, 1H), 8.56 (s, 1H), 7.88 (d, |
| | | J = 8.4 Hz, 1H), 7.80-7.68 (m, 2H), 7.67-7.61 (m, 1H), 7.48-7.35 (m, 1H), |
| | | 7.34-7.27 (m, 1H), 7.20 (d, J = 8.1 Hz, 1H), 7.00-6.89 (m, 1H), 6.52 (s, |
| | | 1H), 6.24-6.09 (m, 2H), 4.40-4.34 (m, 3H), 4.32 (s, 1H), 3.98-3.90 (m, |
| | | 1H), 3.77-3.70 (m, 1H), 3.64 (t, J = 5.7 Hz, 1H), 3.59-3.56 (m, 1H), 3.52- |
| | | 3.38 (m, 4H), 3.16 (s, 6H), 3.08-3.00 (m, 3H), 3.00-2.97 (m, 2H), 2.96 (s, |
| | | 1H), 2.81-2.79 (m, 1H), 2.78 (s, 1H), 2.76 (d, J = 2.9 Hz, 4H), 2.35- 2.24 |
| | | (m, 2H) |
| I-1093 | 906.5 | 12.37-12.17 (m, 1H), 10.98-10.73 (m, 1H), 8.53-8.47 (m, 1H), 7.87-7.78 |
| | | (m, 1H), 7.58-7.46 (m, 1H), 7.42 (dd, J = 5.9, 13.1 Hz, 1H), 7.32-7.24 (m, |
| | | 1H), 7.10-6.98 (m, 2H), 6.66-6.57 (m, 1H), 6.57-6.49 (m, 1H), 6.49-6.39 |
| | | (m, 1H), 6.28 (br s, 1H), 6.27-6.17 (m, 1H), 4.83 (s, 1H), 4.53-4.45 (m, |
| | | 3H), 4.40 (br s, 1H), 3.77-3.66 (m, 2H), 3.27 (br s, 6H), 3.22-3.15 (m, 4H), |
| | | 3.10-3.05 (m, 2H), 2.91-2.86 (m, 2H), 2.78 (br s, 4H), 2.74-2.70 (m, 2H), |
| | | 2.59-2.55 (m, 4H), 2.44 (br s, 1H), 2.40-2.34 (m, 1H), 2.33-2.25 (m, 1H), |
| | | 2.08-1.97 (m, 1H), 1.48 (br s, 3H), 1.46-1.39 (m, 3H) |
| I-1290 | 841.3 | 11.99 (s, 1H), 11.11 (s, 1H), 7.18 (d, J = 8.4 Hz, 1H), 7.00 (d, J = 5.6 Hz, 2H), |
| | | 6.98-6.93 (m, 1H), 6.86 (d, J = 6.0 Hz, 1H), 6.68 (s, 1H), 6.64-6.58 (m, |
| | | 1H), 6.50 (d, J = 2.4Hz, 1H), 6.12 (s, 1H), 5.39 (dd, J = 5.4, 12.6 Hz, 1H), |
| | | 4.06 (s, 2H), 3.72 (s, 3H), 3.63 (s, 3H), 3.37-3.35 (m, 3H), 3.27 (s, 5H), 3.20- |
| | | 3.11 (m, 4H), 3.10-3.01 (m, 2H), 2.98 (s, 3H), 2.95-2.83 (m, 1H), 2.74- |
| | | 2.60 (m, 8H), 2.43 (d, J = 3.2 Hz, 2H), 2.08-1.95 (m, 1H) |
| I-1405 | 866.3 | 11.09 (s, 1H), 8.10 (d, J = 10.4 Hz, 1H), 7.73-7.64 (m, 1H), 7.51 (d, J = 2.0 |
| | | Hz, 1H), 7.39-7.34 (m, 1H), 7.25-7.22 (m, 1H), 7.01-6.92 (m, 3H), 6.70 |
| | | (s, 1H), 6.67-6.62 (m, 1H), 6.18-6.07 (m, 1H), 5.41-5.32 (m, 1H), 4.65- |
| | | 4.59 (m, 2H), 4.36-4.28 (m, 2H), 3.74 (d, J = 4.4 Hz, 3H), 3.66-3.58 (m, |
| | | 5H), 3.29-3.27 (m, 4H), 3.11 (dd, J = 7.2, 14.0 Hz, 4H), 2.92-2.84 (m, 1H), |
| | | 2.70-2.63 (m, 8H), 2.33 (td, J = 2.0, 4.0 Hz, 2H), 2.28-2.24 (m, 1H), 2.04- |
| | | 1.97 (m, 1H) |
| I-1407 | 1052.8 | 12.10-12.00 (m, 1H), 11.09 (s, 1H), 8.15-8.08 (m, 1H), 7.79 (d, J = 2.4 Hz, |
| | | 1H), 7.73-7.65 (m, 1H), 7.36 (dd, J = 2.4, 10.4 Hz, 1H), 7.18 (d, J = 8.4 Hz, |
| | | 1H), 6.99-6.98 (m, 2H), 6.97-6.93 (m, 1H), 6.92-6.81 (m, 1H), 6.68 (s, |
| | | 1H), 6.60 (dd, J = 1.6, 8.4 Hz, 1H), 6.45 (s, 1H), 6.15-6.07 (m, 1H), 5.39- |
| | | 5.35 (m, 1H), 4.67-4.58 (m, 2H), 4.40-4.28 (m, 2H), 3.84 (s, 3H), 3.80 (s, |
| | | 3H), 3.73-3.70 (m, 3H), 3.66-3.58 (m, 5H), 3.26 (s, 8H), 3.17-3.07 (m, |
| | | 4H), 2.96-2.84 (m, 1H), 2.73-2.60 (m, 8H), 2.34-2.32 (m, 2H), 2.27-2.27 |
| | | (m, 1H), 2.05-1.95 (m, 1H) |
| I-1408 | 967.5 | 12.02-12.01 (m, 1H), 11.10 (s, 1H), 8.17-8.08 (m, 1H), 7.74-7.63 (m, 1H), |
| | | 7.17 (d, J = 8.4 Hz, 1H), 6.99 (d, J = 5.2 Hz, 2H), 6.97-6.93 (m, 1H), 6.90- |
| | | 6.81 (m, 1H), 6.68 (s, 1H), 6.60 (d, J = 8.4 Hz, 1H), 6.39 (s, 1H), 6.16-6.06 |
| | | (m, 1H), 5.35-5.35 (m, 1H), 5.38 (dd, J = 5.2, 12.4 Hz, 1H), 4.67-4.59 (m, |
| | | 2H), 4.39-4.27 (m, 2H), 3.73-3.69 (m, 3H), 3.67-3.58 (m, 8H), 3.26 (s, |
| | | 4H), 3.12-3.07 (m, 4H), 2.76-2.62 (m, 8H), 2.55 (s, 5H), 2.37-2.24 (m, |
| | | 2H), 2.03-1.96 (m, 1H), 1.67-1.62 (m, 1H), 0.43-0.42 (m, 2H), 0.33-0.32 |
| | | (m, 2H) |
| I-1409 | 964.5 | 12.09-11.97 (m, 1H), 11.17-11.02 (m, 1H), 8.16-8.06 (m, 1H), 7.74-7.63 |
| | | (m, 1H), 7.31-7.17 (m, 2H), 7.01-6.93 (m, 3H), 6.92-6.83 (m, 1H), 6.80- |
| | | 6.76 (m, 1H), 6.70 (s, 1H), 6.62 (d, J = 7.6 Hz, 1H), 6.06 (d, J = 3.2 Hz, 1H), |
| | | 5.43-5.32 (m, 1H), 5.02 (s, 1H), 4.85-4.77 (m, 2H), 4.66-4.58 (m, 3H), |
| | | 4.38-4.30 (m, 2H), 3.82-3.74 (m, 6H), 3.68-3.64 (m, 1H), 3.62 (s, 3H), |
| | | 3.60-3.58 (m, 1H), 3.11 (dd, J = 7.6, 14.0 Hz, 4H), 2.94-2.86 (m, 1H), 2.77- |
| | | 2.59 (m, 10H), 2.38-2.25 (m, 4H), 2.03-1.96 (m, 1H) |
| I-1410 | 926.3 | 11.99-11.98 (d, J = 7.2 Hz, 1H), 11.10 (s, 1H), 8.15-8.05 (m, 1H), 7.72- |
| | | 7.65 (m, 1H), 7.17 (d, J = 8.4 Hz, 1H), 6.99 (d, J = 5.6 Hz, 2H), 6.97-6.93 |
| | | (m, 1H), 6.90-6.81 (m, 1H), 6.67 (s, 1H), 6.60 (dd, J = 1.6, 8.4 Hz, 1H), 6.36 |
| | | (s, 1H), 6.14-6.06 (m, 1H), 5.38 (dd, J = 5.6, 12.8 Hz, 1H), 4.67-4.57 (m, |
| | | 2H), 4.39-4.27 (m, 2H), 3.74-3.69 (m, 3H), 3.67-3.57 (m, 9H), 3.29-3.23 |
| | | (m, 4H), 3.16-3.06 (m, 4H), 2.96-2.83 (m, 1H), 2.70-2.63 (m, 7H), 2.39- |
| | | 2.31 (m, 2H), 2.26 (s, 1H), 2.04-1.95 (m, 1H), 1.63-1.62 (m, 2H), 1.52 (s, |
| | | 4H) |
| I-1283 | 886.3 | 12.01-11.88 (m, 1H), 11.10 (s, 1H), 8.14-8.08 (m, 1H), 7.72-7.65 (m, 1H), |
| | | 7.17 (d. J = 8.4 Hz, 1H), 6.99 (d, J = 5.6 Hz, 2H), 6.96-6.92 (m, 1H), 6.90- |
| | | 6.81 (m, 1H), 6.67 (s, 1H), 6.62-6.57 (m, 1H), 6.48 (s, 1H), 6.14-6.06 (m, |
| | | 1H), 5.38 (dd, J = 5.6, 12.8 Hz, 1H), 4.67-4.57 (m, 2H), 4.39-4.27 (m, 2H), |
| | | 3.73-3.69 (m, 3H), 3.67-3.57 (m, 5H), 3.26 (s, 4H), 3.20-3.02 (m, 9H), |
| | | 2.89-2.85 (m, 1H), 2.72-2.61 (m, 8H), 2.39-2.31 (m, 2H), 2.26 (s, 1H), |
| | | 2.05-1.96 (m, 1H) |
| I-1268 | 909.2 | 11.10 (s, 1H), 8.11 (d, J = 10.4 Hz, 1H), 7.83 (d, J = 6.4 Hz, 1H), 7.69 (d, J = |
| | | 11.2 Hz, 1H), 7.56 (t, J = 7.2 Hz, 1H), 7.36 (s, 1H), 7.32-7.19 (m, 2H), 7.00- |
| | | 6.92 (m, 2H), 6.68 (s, 1H), 6.66-6.54 (m, 2H), 6.44 (t, J = 6.8 Hz, 1H), 6.13 |
| | | (d, J = 11.6 Hz, 1H), 5.43-5.30 (m, 1H), 4.64-4.59 (m, 2H), 4.39-4.25 (m, |
| | | 2H), 3.75-3.74 (m, 3H), 3.68-3.56 (m, 5H), 3.47-3.39 (m, 2H), 3.27 (s, |
| | | 4H), 3.20-3.03 (m, 4H), 2.95-2.83 (m, 1H), 2.76-2.58 (m, 7H), 2.40-2.23 |
| | | (m, 2H), 2.05-1.94 (m, 1H) |
| I-1276 | 882.3 | 11.10 (s, 1H), 8.31 (d, J = 2.4 Hz, 1H), 8.11 (d, J = 9.6 Hz, 1H), 7.88 (s, 1H), |
| | | 7.69 (d, J = 11.2 Hz, 1H), 7.33-7.13 (m, 2H), 7.06 (d, J = 3.2 Hz, 1H), 7.02- |
| | | 6.92 (m, 3H), 6.71 (s, 1H), 6.67-6.59 (m, 2H), 6.13 (d, J = 11.6 Hz, 1H), |
| | | 5.38 (dd, J = 5.2, 12.8 Hz, 1H), 4.68-4.56 (m, 2H), 4.34-4.30 (m, 2H), 3.77- |
| | | 3.76 (m, 3H), 3.68-3.56 (m, 5H), 3.29-3.26 (m, 2H), 3.18-3.07 (m, 4H), |
| | | 2.97-2.84 (m, 1H), 2.77-2.58 (m, 9H), 2.41-2.23 (m, 3H), 2.06-1.93 (m, |
| | | 1H) |
| I-1277 | 899.3 | 11.10 (s, 1H), 8.11 (d, J = 10.0 Hz, 1H), 7.68 (d, J = 11.2 Hz, 1H), 7.25 (d, J = |
| | | 8.4 Hz, 1H), 7.12-7.02 (m, 1H), 7.01-6.91 (m, 3H), 6.77 (d, J = 1.6 Hz, |
| | | 1H), 6.68 (s, 1H), 6.62 (d, J = 8.8 Hz, 1H), 6.10 (d, J = 11.6 Hz, 1H), 5.38 |
| | | (dd, J = 5.2, 12.8 Hz, 1H), 4.66-4.57 (m, 2H), 4.34-4.25 (m, 2H), 3.91- |
| | | 3.88 (m, 2H), 3.77-3.76 (m, 3H), 3.66-3.55 (m, 5H), 3.28 (s, 4H), 3.18- |
| | | 3.05 (m, 4H), 2.95-2.81 (m, 1H), 2.76-2.59 (m, 8H), 2.56-2.53 (m, 2H), |
| | | 2.36-2.22 (m, 2H), 2.15-2.07 (m, 2H), 2.02-1.98 (m, 1H) |
| I-1461 | 925.3 | 12.05 (d, J = 7.2 Hz, 1H), 11.10 (d, J = 0.8 Hz, 1H), 8.84 (s, 1H), 8.15-8.07 |
| | | (m, 1H), 7.73-7.63 (m, 1H), 7.16 (d, J = 8.4 Hz, 1H), 7.03-6.92 (m, 4H), |
| | | 6.91-6.82 (m, 1H), 6.71 (s, 1H), 6.62 (d, J = 8.4 Hz, 1H), 6.15-6.07 (m, |
| | | 1H), 5.38 (dd, J = 5.2, 12.8 Hz, 1H), 4.66-4.57 (m, 2H), 4.39-4.27 (m, 2H), |
| | | 3.73-3.69 (m, 3H), 3.67-3.57 (m, 6H), 3.26 (s, 4H), 3.17-3.05 (m, 6H), |
| | | 2.94-2.84 (m, 1H), 2.73-2.62 (m, 8H), 2.39-2.23 (m, 2H), 2.05-1.97 (m, |
| | | 1H), 1.25 (d, J = 7.2 Hz, 3H) |
| I-1377 | 873.2 | 12.05-11.80 (m, 1H), 11.32-11.08 (m, 1H), 8.16-8.04 (m, 1H), 7.74-7.64 |
| | | (m, 1H), 7.22-7.05 (m, 4H), 6.91-6.78 (m, 1H), 6.66 (s, 1H), 6.62-6.55 |
| | | (m, 1H), 6.48 (s, 1H), 6.15-6.04 (m, 1H), 5.36 (dd, J = 4.8, 12.8 Hz, 1H), |
| | | 4.68-4.56 (m, 2H), 4.40-4.26 (m, 2H), 3.75-3.69 (m, 3H), 3.67-3.56 (m, |
| | | 2H), 3.27-3.20 (m, 6H), 3.12-3.05 (m, 4H), 2.98-2.84 (m, 4H), 2.70-2.62 |
| | | (m, 8H), 2.33-2.32 (m, 2H), 2.26 (s, 1H), 2.20-2.10 (m, 1H) |
| I-1322 | 868.3 | 11.10 (s, 1H), 8.11 (d, J = 8.4 Hz, 1H), 7.94-7.81 (m, 1H), 7.72 (d, J = 15.6 |
| | | Hz, 1H), 7.68-7.48 (m, 1H), 7.26-7.09 (m, 2H), 7.06-6.89 (m, 4H), 6.77 |
| | | (s, 1H), 6.65 (dd, J = 1.2, 9.2 Hz, 1H), 6.43-6.27 (m, 1H), 5.38 (dd, J = 5.6, |
| | | 12.8 Hz, 1H), 4.69-4.58 (m, 2H), 4.42-4.28 (m, 2H), 3.71 (d, J = 4.8 Hz, |
| | | 3H), 3.68-3.64 (m, 1H), 3.62 (s, 3H), 3.60-3.58 (m, 1H), 3.21-3.08 (m, |
| | | 5H), 2.94 (s, 6H), 2.91-2.84 (m, 1H), 2.78-2.72 (m, 1H), 2.71-2.58 (m, |
| | | 9H), 2.40-2.36 (m, 1H), 2.32-2.28 (m, 2H), 2.04-1.93 (m, 1H) |
| I-1266 | 856.2 | 12.17 (s, 1H), 11.09 (s, 1H), 8.18-8.05 (m, 1H), 7.72-7.63 (m, 1H), 7.39- |
| | | 7.28 (m, 1H), 7.14 (s, 1H), 7.09-6.89 (m, 6H), 6.84 (d, J = 3.2 Hz, 1H), 6.16 |
| | | (d, J = 2.0 Hz, 1H), 5.37 (dd, J = 5.2, 12.8 Hz, 1H), 4.67-4.56 (m, 2H), 4.42- |
| | | 4.29 (m, 2H), 3.68-3.57 (m, 5H), 3.24 (s, 6H), 3.17-2.99 (m, 8H), 2.93- |
| | | 2.83 (m, 1H), 2.75-2.59 (m, 8H), 2.38-2.26 (m, 2H), 2.04-1.95 (m, 1H) |
| I-1389 | 907.5 | 12.03-11.87 (m, 1H), 11.13 (s, 1H), 8.38 (d, J = 8.4 Hz, 1H), 8.16-8.07 (m, |
| | | 2H), 7.88 (dd, J = 7.2, 8.0 Hz, 1H), 7.72-7.64 (m, 1H), 7.42 (d, J = 7.6 Hz, |
| | | 1H), 7.23-7.15 (m, 1H), 7.10 (d, J = 7.6 Hz, 1H), 6.93-6.79 (m, 1H), 6.67 |
| | | (s, 1H), 6.60 (dd, J = 1.6, 8.4 Hz, 1H), 6.53-6.45 (m, 1H), 6.15-6.06 (m, |
| | | 1H), 5.53-5.39 (m, 1H), 4.68-4.56 (m, 2H), 4.39-4.27 (m, 2H), 3.74 (s, |
| | | 3H), 3.59-3.55 (m, 1H), 3.66-3.58 (m, 1H), 3.25-3.22 (m, 8H), 3.20-3.15 |
| | | (m, 2H), 3.14-3.00 (m, 6H), 2.76-2.73 (m, 1H), 2.69-2.65 (m, 6H), 2.39- |
| | | 2.24 (m, 2H), 2.09-2.05 (m, 1H) |
| I-1451 | 917.7 | 12.15 (d, J = 8.8 Hz, 1H), 11.07 (s, 1H), 7.78-7.65 (m, 1H), 7.47-7.34 (m, |
| | | 1H), 7.33-7.13 (m, 4H), 7.01-6.78 (m, 3H), 6.38 (s, 1H), 6.27-6.08 (m, |
| | | 2H), 5.34-5.12 (m, 1H), 4.47-4.22 (m, 4H), 3.69-3.53 (m, 2H), 3.24-2.94 |
| | | (m, 10H), 2.93-2.79 (m, 4H), 2.75-2.56 (m, 5H), 2.39-2.30 (m, 2H), 2.29- |
| | | 2.07 (m, 5H), 2.02-1.83 (m, 2H), 1.41 (s, 6H) |
| I-1452 | 917.7 | 12.16 (d, J = 8.8 Hz, 1H), 11.07 (s, 1H), 7.77-7.65 (m, 1H), 7.48-7.35 (m, |
| | | 1H), 7.34-7.11 (m, 4H), 7.02-6.76 (m, 3H), 6.38 (d, J = 2.4 Hz, 1H), 6.29- |
| | | 6.02 (m, 2H), 5.37-5.04 (m, 1H), 4.44-4.20 (m, 4H), 3.67-3.52 (m, 2H), |
| | | 3.24-2.94 (m, 10H), 2.93-2.78 (m, 4H), 2.74-2.56 (m, 5H), 2.41-2.31 (m, |
| | | 2H), 2.30-2.08 (m, 5H), 2.01-1.83 (m, 2H), 1.41 (d, J = 3.2 Hz, 6H) |
| I-1373 | 906.4 | 12.20 (d, J = 6.8 Hz, 1H), 11.10 (s, 1H), 8.25 (d, J = 8.4 Hz, 1H), 8.16-8.04 |
| | | (m, 1H), 7.72-7.63 (m, 2H), 7.54 (t, J = 7.6 Hz, 1H), 7.48 (d, J = 7.6 Hz, |
| | | 1H), 7.44-7.37 (m, 1H), 7.24 (d, J = 7.8 Hz, 1H), 7.07-6.93 (m, 4H), 6.28- |
| | | 6.23 (m, 1H), 6.21-6.11 (m, 1H), 5.38 (dd, J = 5.6, 12.4 Hz, 1H), 4.70- 4.53 |
| | | (m, 2H), 4.45-4.27 (m, 2H), 3.64 (s, 3H), 3.59 (t, J = 5.6 Hz, 2H), 3.21- |
| | | 3.12 (m, 6H), 3.12-3.08 (m, 2H), 3.05-2.94 (m, 6H), 2.93-2.81 (m, 4H), |
| | | 2.78-2.72 (m, 3H), 2.68-2.59 (m, 2H), 2.36-2.23 (m, 2H), 2.09-1.93 (m, |
| | | 1H) |
| I-1388 | 906.4 | 12.02-11.90 (m, 1H), 11.16 (s, 1H), 8.51 (d, J = 8.0 Hz, 1H), 8.45 (d, J = 3.6 |
| | | Hz, 1H), 8.18 (s, 1H), 8.13-8.09 (m, 1H), 7.71-7.66 (m, 1H), 7.56-7.41 |
| | | (m, 2H), 7.34 (dd, J = 4.8, 7.6 Hz, 1H), 7.22-7.15 (m, 2H), 6.91-6.82 (m, |
| | | 1H), 6.71-6.67 (m, 1H), 6.65-6.59 (m, 1H), 6.52-6.46 (m, 1H), 6.13-6.07 |
| | | (m, 1H), 4.66-4.59 (m, 2H), 4.39-4.28 (m, 2H), 3.74-3.69 (m, 3H), 3.65 (t, |
| | | J = 5.6 Hz, 1H), 3.59 (t, J = 6.0 Hz, 1H), 3.24-3.16 (m, 4H), 3.13-3.05 (m, |
| | | 6H), 3.03 (d, J = 1.2 Hz, 2H), 2.80-2.68 (m, 8H), 2.58-2.54 (m, 4H), 2.37- |
| | | 2.34 (m, 1H), 2.29-2.24 (m, 1H), 2.12 (s, 1H) |
| I-1374 | 895.6 | 12.21-11.98 (m, 1H), 11.11 (s, 1H), 10.98 (s, 1H), 8.16-8.06 (m, 1H), 7.75- |
| | | 7.63 (m, 1H), 7.36-7.23 (m, 1H), 7.15-7.03 (m, 2H), 7.03-6.93 (m, 3H), |
| | | 6.80 (d, J = 7.6 Hz, 1H), 6.67 (s, 1H), 6.40-6.31 (m, 1H), 6.21-6.08 (m, |
| | | 1H), 5.39 (dd, J = 5.2, 12.4 Hz, 1H), 4.68-4.55 (m, 2H), 4.44-4.25 (m, 2H), |
| | | 3.64 (s, 3H), 3.62-3.57 (m, 2H), 3.20-3.11 (m, 8H), 3.11-2.97 (m, 6H), |
| | | 2.92-2.90 (m, 1H), 2.86-2.75 (m, 4H), 2.75-2.62 (m, 4H), 2.42-2.24 (m, |
| | | 2H), 2.01-1.98 (m, 1H) |
| I-1375 | 895.0 | 2.13 (s, 1H), 11.10 (s, 1H), 10.68 (s, 1H), 8.15-8.07 (m, 1H), 7.74-7.63 (m, |
| | | 1H), 7.18 (s, 1H), 7.14-7.08 (m, 1H), 7.06-6.93 (m, 4H), 6.60 (d, J = 8.0 |
| | | Hz, 1H), 6.54 (s, 1H), 6.48 (d, J = 2.4 Hz, 1H), 6.24-6.16 (m, 1H), 5.38 (dd, |
| | | J = 5.6, 12.4 Hz, 1H), 4.62 (q, J = 6.8 Hz, 2H), 4.46-4.29 (m, 2H), 3.69- |
| | | 3.56 (m, 5H), 3.47-3.41 (m, 1H), 3.30-3.26 (m, 1H), 3.25-3.20 (m, 4H), |
| | | 3.18-3.02 (m, 9H), 2.94-2.85 (m, 1H), 2.78-2.75 (m, 3H), 2.74-2.63 (m, |
| | | 4H), 2.39-2.24 (m, 2H), 2.06-1.96 (m, 1H) |
| I-1364 | 858.3 | 12.20 (d, J = 7.6 Hz, 1H), 11.10 (s, 1H), 8.15-8.08 (m, 1H), 8.03-7.92 (m, |
| | | 1H), 7.72-7.66 (m, 1H), 7.45-7.36 (m, 2H), 7.31 (d, J = 8.8 Hz, 1H), 7.01- |
| | | 6.92 (m, 3H), 6.19 (s, 1H), 5.37 (dd, J = 5.2, 12.4 Hz, 1H), 4.63 (m, 2H), 4.43- |
| | | 4.32 (m, 2H), 3.74-3.65 (m, 5H), 3.62 (s, 4H), 3.47-3.41 (m, 3H), 3.17- |
| | | 3.09 (m, 6H), 2.88 (dd, J = 5.2, 16.4 Hz, 1H), 2.75-2.58 (m, 8H), 2.53 (s, |
| | | 1H), 2.41-2.26 (m, 2H), 2.05-1.96 (m, 1H) |
| I-1376 | 922.5 | 12.18-11.89 (m, 1H), 11.21-10.94 (m, 1H), 8.16-8.04 (m, 1H), 7.75-7.60 |
| | | (m, 1H), 7.40-7.09 (m, 2H), 7.01-6.92 (m, 4H), 6.92-6.85 (m, 1H), 6.81 |
| | | (s, 1H), 6.54 (d, J = 3.2 Hz, 1H), 6.18-6.07 (m, 1H), 5.37 (dd, J = 5.2, 12.4 |
| | | Hz, 1H), 4.62 (q, J = 7.2 Hz, 2H), 4.41-4.28 (m, 2H), 3.68-3.56 (m, 5H), |
| | | 3.28 (s, 3H), 3.21-3.00 (m, 10H), 2.93-2.84 (m, 1H), 2.74-2.62 (m, 9H), |
| | | 2.36-2.24 (m, 2H), 2.05-1.94 (m, 1H) |
| I-1358 | 886.3 | 12.14-12.02 (m, 1H), 11.16-11.04 (m, 1H), 8.32 (s, 1H), 8.11 (d, J = 11.6 |
| | | Hz, 1H), 7.73-7.64 (m, 1H), 7.08 (s, 1H), 6.98 (d, J = 4.8 Hz, 2H), 6.97- |
| | | 6.90 (m, 2H), 6.53-6.47 (m, 1H), 6.20-6.09 (m, 1H), 5.37 (dd, J = 5.2, 12.8 |
| | | Hz, 1H), 4.66-4.59 (m, 2H), 4.40-4.29 (m, 2H), 3.82 (d, J = 3.6 Hz, 3H), |
| | | 3.68-3.63 (m, 1H), 3.61 (s, 3H), 3.60-3.56 (m, 1H), 3.19-3.05 (m, 10H), |
| | | 2.93-2.85 (m, 1H), 2.76-2.59 (m, 6H), 2.39-2.31 (m, 2H), 2.31-2.22 (m, |
| | | 1H), 2.16-2.08 (m, 2H), 2.03-1.97 (m, 1H), 1.93-1.83 (m, 4H) |
| I-1390 | 834.7 | 12.05-11.84 (m, 1H), 11.07-10.96 (m, 1H), 8.16-8.06 (m, 1H), 7.97 (d, J = |
| | | 2.0 Hz, 1H), 7.74-7.63 (m, 1H), 7.17 (d, J = 8.4 Hz, 1H), 6.91-6.80 (m, |
| | | 2H), 6.66 (s, 1H), 6.60 (d, J = 8.4 Hz, 1H), 6.48 (s, 1H), 6.19-6.01 (m, 1H), |
| | | 5.68 (dd, J = 5.2, 12.4 Hz, 1H), 4.67-4.56 (m, 2H), 4.39-4.26 (m, 2H), 3.73- |
| | | 3.69 (m, 3H), 3.59-3.57 (m, 1H), 3.67-3.55 (m, 1H), 3.23-3.20 (m, 6H), |
| | | 3.14-3.06 (m, 4H), 2.92-2.90 (m, 2H), 2.90-2.82 (m, 1H), 2.78-2.72 (m, |
| | | 2H), 2.70-2.63 (m, 4H), 2.62-2.60 (m, 4H), 2.36-2.26 (m, 2H), 2.10-2.01 |
| | | (m, 1H) |
| I-1356 | 856.6 | 12.08-12.24 (m, 1 H) 11.09 (s, 1 H) 8.64 (s, 1 H) 8.11-8.16 (m, 1 H) 7.90 |
| | | (br d, J = 8.13 Hz, 1 H) 7.80 (br d, J = 7.25 Hz, 1 H) 7.65-7.72 (m, 1 H) 7.45- |
| | | 7.53 (m, 1 H) 7.31 (dt, J = 5.32, 2.72 Hz, 1 H) 6.87-7.07 (m, 3 H) 6.17 (br s, 1 |
| | | H) 5.38 (dd, J = 12.63, 5.25 Hz, 1 H) 4.56-4.69 (m, 2 H) 4.31-4.45 (m, 2 H) |
| | | 3.57-3.70 (m, 5 H) 3.04-3.25 (m, 12 H) 2.84-2.94 (m, 1 H) 2.65-2.79 (m, |
| | | 4 H) 2.61 (br s, 2 H) 2.27-2.39 (m, 3 H) 1.97-2.05 (m, 1 H) 1.70-1.96 (m, |
| | | 4 H) |
| I-1357 | 886.4 | 12.13-11.90 (m, 1H), 11.20-10.99 (m, 1H), 8.21-8.16 (m, 1H), 8.15-8.07 |
| | | (m, 1H), 7.72-7.65 (m, 1H), 7.49-7.44 (m, 1H), 7.29-7.16 (m, 1H), 6.99 |
| | | (d, J = 4.8 Hz, 2H), 6.96-6.90 (m, 1H), 6.76-6.66 (m, 1H), 6.17-6.06 (m, |
| | | 1H), 5.37 (dd, J = 5.2, 12.4 Hz, 1H), 4.68-4.58 (m, 2H), 4.41-4.27 (m, 2H), |
| | | 3.86-3.76 (m, 3H), 3.69-3.62 (m, 2H), 3.61-3.56 (m, 3H), 3.21-2.97 (m, |
| | | 12H), 2.95-2.83 (m, 1H), 2.76-2.60 (m, 5H), 2.37-2.32 (m, 1H), 2.30- |
| | | 2.25 (m, 1H), 2.22-2.12 (m, 2H), 2.06-1.95 (m, 1H), 1.90-1.77 (m, 4H) |
| I-1362 | 886.3 | 12.09 (d, J = 7.2 Hz, 1H), 11.09 (s, 1H), 8.27-8.11 (m, 1H), 7.74-7.65 (m, |
| | | 2H), 7.04-6.89 (m, 5H), 6.53 (d, J = 2.0 Hz, 1H), 6.19-6.09 (m, 1H), 5.37 |
| | | (dd, J = 5.2, 12.4 Hz, 1H), 4.69-4.55 (m, 2H), 4.40-4.27 (m, 2H), 3.88- |
| | | 3.80 (m, 3H), 3.68-3.62 (m, 2H), 3.61-3.56 (m, 3H), 3.16-3.02 (m, 10H), |
| | | 2.96-2.83 (m, 2H), 2.77-2.56 (m, 6H), 2.37-2.32 (m, 1H), 2.30-2.24 (m, |
| | | 1H), 2.18 (t, J = 10.4 Hz, 2H), 2.04-1.97 (m, 1H), 1.96-1.89 (m, 2H), 1.89- |
| | | 1.78 (m, 2H) |
| I-1348 | 860.6 | 12.02 (d, J = 6.4 Hz, 1H), 11.09 (s, 1H), 8.14-8.10 (m, 1H), 7.72-7.67 (m, |
| | | 1H), 7.00-6.95 (m, 2H), 6.91 (dd, J = 3.2, 6.0 Hz, 1H), 6.88-6.80 (m, 2H), |
| | | 6.13-5.96 (m, 2H), 5.37 (dd, J = 5.2, 12.4 Hz, 1H), 4.67-4.60 (m, 2H), 4.34- |
| | | 4.26 (m, 2H), 3.66-3.63 (m, 1H), 3.59 (s, 5H), 3.14-3.04 (m, 7H), 2.95- |
| | | 2.84 (m, 1H), 2.74-2.54 (m, 15H), 2.43-2.32 (m, 4H), 2.29-2.19 (m, 2H), |
| | | 2.09-1.97 (m, 2H), 1.63-1.52 (m, 1H) |
| I-1353 | 863.3 | 11.94-11.89 (m, 1H), 11.09 (s, 1H), 8.13-8.08 (m, 1H), 7.71-7.65 (m, 1H), |
| | | 6.99-6.95 (m, 2H), 6.93-6.88 (m, 1H), 6.73 (br s, 1H), 6.34-6.28 (m, 1H), |
| | | 6.05 (br s, 1H), 5.36 (br dd, J = 5.4, 12.6 Hz, 1H), 4.62 (q, J = 6.5 Hz, 2H), |
| | | 4.36-4.23 (m, 2H), 3.67-3.62 (m, 1H), 3.60-3.56 (m, 6H), 3.22 (br s, 4H), |
| | | 3.12-3.04 (m, 7H), 2.92-2.85 (m, 1H), 2.75-2.64 (m, 4H), 2.59-2.54 (m, |
| | | 7H), 2.37-2.25 (m, 4H), 2.05-1.84 (m, 4H), 1.73-1.63 (m, 2H) |
| I-1395 | 883.4 | 11.95 (d, J = 7.2 Hz, 1H), 11.13-10.96 (m, 1H), 8.17-8.08 (m, 2H), 7.75- |
| | | 7.62 (m, 2H), 7.52-7.40 (m, 2H), 7.18 (d, J = 8.4 Hz, 1H), 6.94-6.75 (m, |
| | | 2H), 6.73-6.65 (m, 1H), 6.64-6.57 (m, 1H), 6.52-6.45 (m, 1H), 6.18-6.04 |
| | | (m, 1H), 5.83-5.15 (m, 1H), 4.69-4.56 (m, 2H), 4.42-4.25 (m, 2H), 3.77- |
| | | 3.69 (m, 3H), 3.68-3.56 (m, 2H), 3.27-2.99 (m, 13H), 2.93-2.81 (m, 1H), |
| | | 2.75-2.57 (m, 9H), 2.40-2.31 (m, 2H), 2.12-2.03 (m, 1H) |
| I-1370 | 896.5 | 12.06 (dd, J = 1.2, 4.4 Hz, 1H), 11.09 (s, 1H), 8.16-8.04 (m, 1H), 7.74-7.62 |
| | | (m, 1H), 7.18-7.07 (m, 1H), 7.03-6.76 (m, 5H), 6.46 (d, J = 17.6 Hz, 2H), |
| | | 6.20-6.08 (m, 1H), 5.44-5.29 (m, 1H), 4.68-4.56 (m, 2H), 4.27 (d, J = 0.8 |
| | | Hz, 2H), 3.69-3.56 (m, 5H), 3.22-3.03 (m, 13H), 2.95-2.84 (m, 1H), 2.76- |
| | | 2.58 (m, 9H), 2.40-2.29 (m, 2H), 2.06-1.94 (m, 1H), 1.76-1.62 (m, 1H), |
| | | 0.78-0.65 (m, 4H) |
| I-1355 | 856.3 | 12.27 (br d, J = 5.9 Hz, 1H), 11.10 (s, 1H), 8.79 (br s, 1H), 8.11 (d, J = 11.9 |
| | | Hz, 1H), 8.02 (dd, J = 1.8, 8.0 Hz, 1H), 7.69 (d, J = 11.9 Hz, 1H), 7.42 (d, J = |
| | | 8.0 Hz, 1H), 7.17-7.06 (m, 1H), 6.98 (d, J = 4.6 Hz, 2H), 6.94 (q, J = 4.5 Hz, |
| | | 1H), 6.87 (br s, 1H), 6.19 (br s, 1H), 5.37 (br dd, J = 5.2, 12.4 Hz, 1H), 4.62 |
| | | (q, J = 7.0 Hz, 2H), 4.42-4.31 (m, 2H), 3.66 (br t, J = 5.8 Hz, 1H), 3.61 (s, |
| | | 4H), 3.26-3.18 (m, 2H), 3.15-3.01 (m, 9H), 2.95-2.85 (m, 1H), 2.79-2.68 |
| | | (m, 2H), 2.67-2.57 (m, 4H), 2.38-2.27 (m, 2H), 2.18 (br t, J = 10.6 Hz, 2H), |
| | | 2.05-1.97 (m, 1H), 1.94-1.76 (m, 4H) |
| I-1365 | 857.2 | 12.31 (d, J = 7.2 Hz, 1H), 11.09 (s, 1H), 8.19 (d, J = 8.8 Hz, 1H), 8.15-8.07 |
| | | (m, 1H), 7.78-7.72 (m, 1H), 7.72-7.66 (m, 1H), 7.61-7.52 (m, 1H), 7.33 |
| | | (d, J = 2.8 Hz, 1H), 6.99 (d, J = 4.8 Hz, 2H), 6.97-6.92 (m, 1H), 6.22 (d, J = |
| | | 4.0 Hz, 1H), 5.38 (dd, J = 5.2, 12.4 Hz, 1H), 4.63 (q, J = 7.2 Hz, 2H), 4.49- |
| | | 4.33 (m, 2H), 3.67 (s, 2H), 3.62 (s, 3H), 3.26-3.16 (m, 4H), 3.15-3.08 (m, |
| | | 6H), 3.07-2.97 (m, 2H), 2.96-2.73 (m, 2H), 2.73-2.58 (m, 4H), 2.40-2.29 |
| | | (m, 2H), 2.27-2.17 (m, 2H), 2.06-1.83 (m, 5H) |
| I-1383 | 830.2 | 12.05-11.86 (m, 1H), 10.91 (s, 1H), 8.13-8.08 (m, 1H), 7.77-7.59 (m, 1H), |
| | | 7.33-7.24 (m, 2H), 7.23-7.12 (m, 3H), 6.93-6.79 (m, 1H), 6.66 (s, 1H), |
| | | 6.60 (d, J = 8.4 Hz, 1H), 6.47 (d, J = 2.4 Hz, 1H), 6.16-6.03 (m, 1H), 4.69- |
| | | 4.56 (m, 2H), 4.40-4.22 (m, 2H), 3.74-3.69 (m, 3H), 3.65 (s, 1H), 3.60- |
| | | 3.55 (m, 1H), 3.28-3.14 (m, 7H), 3.12-3.05 (m, 4H), 2.84-2.76 (m, 2H), |
| | | 2.66 (s, 6H), 2.49-2.24 (m, 5H), 2.13-2.00 (m, 2H), 1.42 (s, 3H) |
| I-1361 | 887.2 | 12.08-11.89 (m, 1H), 11.19-10.99 (m, 1H), 8.17-8.03 (m, 1H), 7.74-7.64 |
| | | (m, 1H), 7.57 (d, J = 8.0 Hz, 1H), 7.03-6.84 (m, 4H), 6.54 (s, 1H), 6.49- |
| | | 6.42 (m, 1H), 6.15-6.06 (m, 1H), 5.38 (dd, J = 5.6, 12.8 Hz, 1H), 4.69-4.56 |
| | | (m, 2H), 4.40-4.26 (m, 2H), 3.84-3.74 (m, 3H), 3.68-3.53 (m, 9H), 3.26- |
| | | 2.97 (m, 10H), 2.95-2.84 (m, 1H), 2.78-2.61 (m, 8H), 2.38-2.26 (m, 2H), |
| | | 2.05-1.96 (m, 1H) |
| I-1366 | 894.5 | 12.20-12.08 (m, 1H), 11.10 (s, 1H), 11.05-10.97 (m, 1H), 8.16-8.07 (m, |
| | | 1H), 7.75-7.63 (m, 1H), 7.58-7.46 (m, 2H), 7.24 (d, J = 8.0 Hz, 1H), 7.08- |
| | | 6.85 (m, 5H), 6.21 (s, 1H), 6.16 (d, J = 2.4 Hz, 1H), 5.38 (dd, J = 5.2, 12.4 |
| | | Hz, 1H), 4.69-4.57 (m, 2H), 4.45-4.31 (m, 2H), 3.70-3.62 (m, 2H), 3.61 |
| | | (s, 3H), 3.17-3.04 (m, 10H), 2.93-2.83 (m, 1H), 2.80-2.58 (m, 7H), 2.35 |
| | | (dd, J = 2.4, 13.9 Hz, 2H), 2.23-2.13 (m, 2H), 2.06-1.96 (m, 3H), 1.84- |
| | | 1.70 (m, 2H) |
| I-1369 | 913.3 | 12.60-12.33 (m, 1H), 11.09 (s, 1H), 8.20-8.05 (m, 1H), 7.98-7.86 (m, 1H), |
| | | 7.76-7.51 (m, 4H), 7.31-7.17 (m, 1H), 7.03-6.86 (m, 3H), 6.30-6.07 (m, |
| | | 1H), 5.43-5.28 (m, 1H), 4.73-4.55 (m, 2H), 4.48-4.25 (m, 2H), 3.74-3.66 |
| | | (m, 2H), 3.66-3.64 (m, 1H), 3.62 (s, 3H), 3.29-3.22 (m, 2H), 3.17-3.08 |
| | | (m, 6H), 2.96-2.83 (m, 2H), 2.75-2.63 (m, 10H), 2.61-2.57 (m, 2H), 2.41- |
| | | 2.26 (m, 2H), 2.07-1.95 (m, 1H) |
| I-1372 | 934.2 | 12.16 (s, 1H), 11.09 (s, 1H), 8.17-8.03 (m, 1H), 7.72-7.63 (m, 1H), 7.57 (s, |
| | | 1H), 7.32 (s, 2H), 7.06-6.90 (m, 4H), 6.35 (s, 1H), 6.21-6.09 (m, 1H), 5.38 |
| | | (dd, J = 5.6, 12.4 Hz, 1H), 4.61 (q, J = 6.8 Hz, 2H), 4.48-4.19 (m, 2H), 3.71- |
| | | 3.53 (m, 5H), 3.34-3.32 (m, 3H), 3.21-2.95 (m, 10H), 2.93-2.84 (m, 1H), |
| | | 2.79-2.56 (m, 12H), 2.37-2.23 (m, 2H), 2.06-1.96 (m, 1H) |
| I-1359 | 887.8 | 12.23-12.03 (m, 1H), 11.09 (s, 1H), 8.17-8.04 (m, 1H), 7.74-7.61 (m, 1H), |
| | | 7.34 (s, 1H), 7.31-7.22 (m, 1H), 7.06-6.87 (m, 3H), 6.68 (s, 1H), 6.22- |
| | | 6.08 (m, 1H), 5.38 (dd, J = 5.6, 12.8 Hz, 1H), 4.68-4.57 (m, 2H), 4.43- 4.29 |
| | | (m, 2H), 3.93-3.83 (m, 3H), 3.71-3.56 (m, 5H), 3.20-3.01 (m, 12H), 2.97- |
| | | 2.87 (m, 2H), 2.70-2.60 (m, 4H), 2.35-2.14 (m, 4H), 2.05-1.91 (m, 5H) |
| I-1398 | 857.3 | 11.93 (d, J = 8.8 Hz, 1H), 10.45 (s, 1H), 8.17-8.06 (m, 1H), 8.03 (s, 1H), 7.74- |
| | | 7.60 (m, 2H), 7.41 (t, J = 8.0 Hz, 1H), 7.15 (d, J = 8.4 Hz, 1H), 7.05 (d, J = 7.6 |
| | | Hz, 1H), 6.91-6.76 (m, 1H), 6.63 (s, 1H), 6.57 (d, J = 7.6 Hz, 1H), 6.47 (s, |
| | | 1H), 6.15-6.01 (m, 1H), 4.69-4.53 (m, 4H), 4.41-4.25 (m, 2H), 3.90-3.87 |
| | | (m, 2H), 3.69 (d, J = 4.0 Hz, 3H), 3.65-3.63 (m, 1H), 3.61-3.58 (m, 1H), |
| | | 3.18 (s, 6H), 3.13-3.04 (m, 5H), 2.87-2.84 (m, 2H), 2.80-2.71 (m, 2H), |
| | | 2.65 (s, 4H), 2.33-2.32 (m, 2H), 2.30-2.22 (m, 1H). |
| I-1352 | 873.3 | 12.35-12.21 (m, 1H), 11.09 (s, 1H), 8.16 (d, J = 6.0 Hz, 1H), 7.73 (d, J = 6.0 |
| | | Hz, 1H), 7.50-7.41 (m, 1H), 6.99 (d, J = 5.2 Hz, 2H), 6.98-6.88 (m, 2H), |
| | | 6.44 (d, J = 3.2 Hz, 1H), 6.26 (d, J = 7.2 Hz, 1H), 6.19-6.12 (m, 1H), 5.61 (d, |
| | | J = 2.0 Hz, 1H), 5.38 (dd, J = 5.2, 12.8 Hz, 1H), 4.63 (q, J = 7.2 Hz, 2H), 4.39 |
| | | (d, J = 6.0 Hz, 2H), 3.69-3.63 (m, 1H), 3.61 (s, 3H), 3.60-3.57 (m, 1H), 3.37- |
| | | 3.34 (m, 4H), 3.21-2.98 (m, 10H), 2.95-2.86 (m, 1H), 2.78-2.60 (m, 8H), |
| | | 2.36-2.25 (m, 2H), 2.06-1.96 (m, 1H) |
| I-1382 | 857.3 | 11.93 (d, J = 8.0 Hz, 1H), 11.09 (s, 1H), 8.16-8.06 (m, 1H), 7.72-7.65 (m, |
| | | 2H), 7.56 (d, J = 7.2 Hz, 1H), 7.40-7.29 (m, 1H), 7.17 (d, J = 8.4 Hz, 1H), |
| | | 6.92-6.79 (m, 1H), 6.66 (s, 1H), 6.59 (dd, J = 1.6, 8.8 Hz, 1H), 6.48 (s, 1H), |
| | | 6.14-6.07 (m, 1H), 4.66-4.56 (m, 3H), 4.38-4.27 (m, 2H), 3.73-3.69 (m, |
| | | 3H), 3.67-3.56 (m, 3H), 3.24 (s, 6H), 3.16-3.08 (m, 7H), 2.79-2.72 (m, |
| | | 3H), 2.69-2.64 (m, 5H), 2.33-2.32 (m, 2H), 2.29-2.16 (m, 2H). |

^{a}The reductive amination was run under standard techniques anywhere from 0-40° C. for 1-3 hrs. Other bases, such as AcONa, and reducing agents, such as NaBH(Oac)₃, could be employed. 4 Å molecular sieves also could be added to promote the reaction when applicable. Final compounds were purified via standard techniques including prep-HPLC and other chromatography techniques. Standard deprotection conditions such as HCl/dioxane or TFA/DCM could also be employed to deprotect final products when applicable.
^{b}LC-MS (ESI⁺) m/z reported as (M/2 + H)+.
^{c}LC-MS (ESI⁺) m/z reported as (M + 23)⁺.
^{d}Ketone instead of aldehyde used for the reductive amination.

### Example 2 (Method 2): Synthesis of 6-(1-Acetyl-1,2,5,6-tetrahydropyridin-3-yl)-4-(4-(1-(1-(2,6-dioxopiperidin-3-yl)-3-methyl-2-oxo-2,3-dihydro-1H-benzo[d]1734midazole-4-yl)piperidin-4-yl)-2-methylphenyl)-7-fluoro-N,N-dim ethyl-1H-indole-2-carboxamide (1-455)

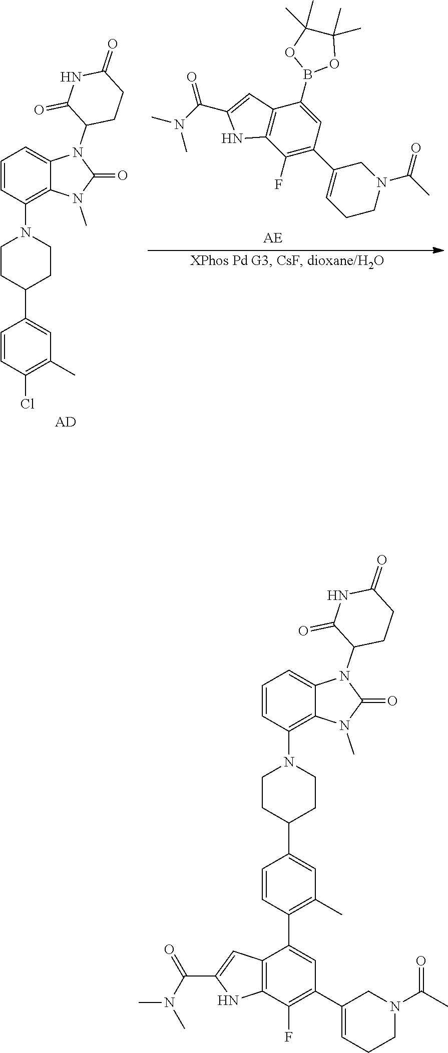

A mixture of 3-(4-(4-(4-chloro-3-methylphenyl)piperidin-1-yl)-3-methyl-2-oxo-2,3-dihydro-1H-benzo[d]1735midazole-1-yl)piperidine-2,6-dione (750 mg, 1.61 mmol, Intermediate AD), 6-(1-acetyl-1,2,5,6-tetrahydropyridin-3-yl)-7-fluoro-N,N-dimethyl-4-(4,4,5,5-tetramethyl-1,3,2-dioxaborolan-2-yl)-1H-indole-2-carboxamide (1.17 g, 2.57 mmol, Intermediate AE), Xphos Pd G3 (136 mg, 161 μmol), and CsF (732 mg, 4.82 mmol, 18.4 μL) in dioxane (5 mL) and H₂O (1 mL) was degassed and purged with N₂ 3 times. Then the mixture was stirred at 80° C. for 2 hrs under N₂ atmosphere. On completion, the reaction mixture was purified by reverse phase HPLC (column: Phenomenex luna C18 150×25 mm 10 um; mobile phase: [water (FA)—can]: gradient: 42%-72% B over 10 min) to give the title compound (700 mg, 54% yield) as a white solid. LC-MS (ESI⁺) m/z 760.5 (M+H)⁺; ¹H NMR (400 MHz, DMSO-d₆) δ=12.-5-11.98 (m, 1H), 11.09 (s, 1H), 7.-7-7.16 (m, 3H), 7.-6-6.93 (m, 2H), 6.89 (d, J=6.4 Hz, 2H), 6.39 (d, J=2.0 Hz, 1H), 6.-0-6.10 (in, 1H), 5.-6-5.30 (m, 1H), 4.-1-4.27 (m, 2H), 3.70 (s, 3H), 3.61 (td, J=5.6, 11.2 Hz, 2H), 3.25 (d, J=11.2 Hz, 3H), 3.-6-3.00 (m, 5H), 2.-4-2.83 (m, 3H), 2.-6-2.59 (in, 3H), 2.-8-2.24 (m, 2H), 2.-0-2.14 (m, 3H), 2.07 (d, J=5.2 Hz, 3H), 1.96 (br d, J=2.8 Hz, 5H).

TABLE 5
Compounds synthesized via Method 2, the cross coupling of the corresponding chlorides and
boronic acids

| | | | LCMS | |
|---|---|---|---|---|
| | | Boronic | (ESI+) m/z | |
| I-# | Chloride | Acid | (M + H)⁺ | ¹H NMR (400 MHZ, DMSO-d₆) δ |
| I-146 | AU | AJ | 845.5 | 10.79 (br s, 1 H), 8.−1-8.24 (m, 1 H), 8.12 (s, 1 H), 7.69 |
| | | | | (d, J = 2.4 Hz, 1 H), 7.−9-7.27 (m, 1 H), 7.15 (t, J = 7.6 |
| | | | | Hz, 1 H), 7.−1-7.02 (m, 1 H), 6.−7-6.76 (m, 2 H), 6.61 |
| | | | | (d, J = 7.6 Hz, 1 H), 6.−3-6.42 (m, 1 H), 6.25 (br d, J = |
| | | | | 13.2 Hz, 1 H), 4.63 (br t, J = 6.8 Hz, 2 H), 4.−1-4.26 |
| | | | | (m, 5 H), 4.16 (q, J = 6.8 Hz, 2 H), 3.−1-3.83 (m, 1 H), |
| | | | | 3.76 (br dd, J = 5.2, 11.2 Hz, 1 H), 3.65 (br d, J = 13.6 |
| | | | | Hz, 1 H), 3.−2-3.55 (m, 2 H), 3.26 (br s, 3 H), 3.−8-3.06 |
| | | | | (m, 7 H), 3.−6-2.93 (m, 4 H), 2.59 (br d, J = 5.2 |
| | | | | Hz, 2 H), 2.52 (br s, 2 H), 2.45 (br s, 2 H), 2.31 (br d, J = |
| | | | | 2.8 Hz, I H), 2.−6-2.12 (m, 4 H), 2.02 (td, J = 4.8, 8.3 |
| | | | | Hz, 1 H), 1.−6-1.69 (m, 3 H), 1.−3-0.89 (m, 2 H) |
| I-356 | AX | AW | 778.3 | 12.−4-11.66 (m, 1 H), 11.10 (br s, 1 H), 7.29 (d, J = 7.6 |
| | | | | Hz, 1 H), 7.09 (s, 1 H), 7.−5-6.88 (m, 5 H), 6.48 (d, J = |
| | | | | 3.2 Hz, 1 H), 5.38 (br dd, J = 5.2, 12.4 Hz, 1 H), 4.48 (br |
| | | | | t, J = 12.4 Hz, 1 H), 3.87 (br d, J = 14.0 Hz, 1 H), 3.74 |
| | | | | (d, J = 19.2 Hz, 6 H), 3.−0-3.23 (m, 3 H), 3.−2-2.97 |
| | | | | (m, 7 H), 2.−5-2.85 (m, 3 H), 2.−1-2.57 (m, 4 H), |
| | | | | 2.−6-1.92 (m, 9 H), 1.−0-1.73 (m, 2 H), 1.−6-1.40 (m, 1 H) |
| I-452 | AQ | AR | 820.3 | 12.13 (d, J = 7.6 Hz, I H), 10.86 (s, 1 H), 7.−7-7.66 (m, |
| | | | | 1 H), 7.55 (d, J = 8.4 Hz, 2 H), 7.−8-7.34 (m, 1 H), 7.10 |
| | | | | (d, J = 8.4 Hz, 2 H), 7.−3-6.91 (m, 1 H), 6.87 (s, 1 H), |
| | | | | 6.68 (d, J = 8.0 Hz, 1 H), 6.59 (d, J = 14.4 Hz, 1 H), |
| | | | | 6.−4-6.10 (m, 2 H), 5.14 (d, J = 6.8 Hz, 1 H), 4.−1-4.33 |
| | | | | (m, 3 H), 4.−3-4.23 (m, 2 H), 3.82 (s, 3 H), 3.65 (t, J = |
| | | | | 5.6 Hz, 1 H), 3.58 (t, J = 5.6 Hz, 1 H), 3.18 (s, 6 H), |
| | | | | 3.−6-3.06 (m, 6 H), 3.−6-3.03 (m, 1 H), 3.−3-2.91 (m, |
| | | | | 3 H), 2.−7-2.75 (m, 1 H), 2.55 (dd, J = 4.0, 13.6 Hz, |
| | | | | 1 H), 2.−9-2.21 (m, 2 H), 2.−0-2.07 (m, 1 H), 1.93 (dq, |
| | | | | J = 4.4, 12.8 Hz, 1 H) |
| I-454 | AP | AE | 746.3 | 12.−3-12.08 (m, 1 H), 11.−5-10.93 (m, 1 H), 7.−8- |
| | | | | 7.60 (m, 2 H), 7.46 (br d, J = 7.6 Hz, 2 H), 7.−9-6.96 |
| | | | | (m, 3 H), 6.−2-6.86 (m, 2 H), 6.18 (br d, J = 6.4 Hz, |
| | | | | 1 H), 5.−1-5.31 (m, 1 H), 4.35 (br d, J = 7.6 Hz, 2 H), |
| | | | | 3.−3-3.67 (m, 3 H), 3.−5-3.59 (m, 2 H), 3.−7-3.22 (m, |
| | | | | 4 H), 3.−3-3.01 (m, 3 H), 2.−4-2.85 (m, 3 H), 2.60 (br |
| | | | | s, 4 H), 2.−0-2.37 (m, 1 H), 2.−3-2.23 (m, 1 H), |
| | | | | 2.−1-2.06 (m, 3 H), 2.02 (br s, 5 H) |
| I-475 | CO | CQ | 826.0 | 11.−7-10.88 (m, 1 H), 8.04 (d, J = 1.6 Hz, 1 H), |
| | | | | 7.−0-7.82 (m, 2 H), 7.−4-7.71 (m, 1 H), 7.−8-7.54 (m, 1 H), |
| | | | | 7.−3-7.36 (m, 1 H), 7.−7-7.14 (m, 1 H), 7.−2-7.09 (m, |
| | | | | 1 H), 7.−7-7.01 (m, 2 H), 6.−9-6.91 (m, 2 H), 6.20 (s, |
| | | | | 1 H), 5.−8-5.32 (m, 1 H), 4.55 (br s, 2 H), 4.37 (br d, J = |
| | | | | 6.4 Hz, 2 H), 4.−1-3.91 (m, 2 H), 3.−8-3.58 (m, 2 H), |
| | | | | 3.35 (br s, 3 H), 3.−3-2.97 (m, 8 H), 2.87 (br d, J = 11.6 |
| | | | | Hz, 3 H), 2.−9-2.68 (m, 2 H), 2.−5-2.59 (m, 1 H), |
| | | | | 2.−4-2.32 (m, 2 H), 2.−3-1.97 (m, 1 H), 1.−3-1.80 (m, 4 H) |
| I-492 | CO | CT | 854.4 | 11.10 (s, 1 H), 8.03 (s, 1 H), 7.85 (br d, J = 7.6 Hz, 1 H), |
| | | | | 7.−2-7.79 (m, 1 H), 7.−4-7.72 (m, 1 H), 7.−6-7.52 (m, |
| | | | | 1 H), 7.−3-7.37 (m, 1 H), 7.06 (br d, J = 8.8 Hz, 1 H), |
| | | | | 7.−0-6.96 (m, 3 H), 6.91 (br d, J = 4.0 Hz, 2 H), |
| | | | | 6.−1-6.17 (m, 1 H), 5.−9-5.34 (m, 1 H), 4.−5-4.49 (m, 2 H), |
| | | | | 4.−9-4.34 (m, 2 H), 3.−0-3.78 (m, 3 H), 3.−8-3.64 (m, |
| | | | | 1 H), 3.59 (s, 3 H), 3.00 (br d, J = 7.2 Hz, 6 H), 2.−0- |
| | | | | 2.85 (m, 2 H), 2.−8-2.73 (m, 3 H), 2.−0-2.63 (m, 6 H), |
| | | | | 2.59 (br s, 2 H), 2.−1-1.98 (m, I H), 1.−2-1.88 (m, 2 H), |
| | | | | 1.60 (br d, J = 0.8 Hz, 2 H), 1.35 (br dd, J = 1.6, 9.6 Hz, |
| | | | | 2 H) |
| I-510 | BD | AX | 786.1 | 11.60 (br s, 1 H), 11.10 (brs, 1 H), 8.11 (br d, J = 9.2 Hz, |
| | | | | 1 H), 7.69 (br d, J = 10.4 Hz, I H), 7.37 (br s, 1 H), 7.27 |
| | | | | (br d, J = 7.6 Hz, 1 H), 7.−1-7.05 (m, 1 H), 7.−4-6.95 |
| | | | | (m, 3 H), 6.−3-6.82 (m, 2 H), 6.20 (br s, 1 H), |
| | | | | 6.−3-6.03 (m, 1 H), 5.37 (br dd, J = 4.8, 12.4 Hz, 1 H), 4.62 |
| | | | | (q, J = 6.8 Hz, 2 H), 4.−3-4.25 (m, 2 H), 3.72 (br d, J = |
| | | | | 14.8 Hz, 6 H), 3.−7-3.58 (m, 2 H), 3.26 (br d, J = 10.4 |
| | | | | Hz, 2 H), 3.09 (br d, J = 4.4 Hz, 2 H), 2.89 (br s, 3 H), |
| | | | | 2.−0-2.60 (m, 3 H), 2.−3-2.20 (m, 2 H), 1.98 (br s, 5 H) |
| I-535 | AD | AW | 762.4 | 12.03 (s, 1 H), 11.09 (s, 1 H), 7.−2-7.21 (m, 3 H), |
| | | | | 7.−4-6.96 (m, 2 H), 6.−3-6.86 (m, 2 H), 6.38 (s, 1 H), 5.37 |
| | | | | (dd, J = 5.2, 12.4 Hz, 1 H), 4.−4-4.40 (m, 1 H), |
| | | | | 3.−0-3.81 (m, 1 H), 3.70 (s, 3 H), 3.25 (d, J = 11.6 Hz, 3 H), |
| | | | | 3.−5-3.02 (m, 6 H), 2.−3-2.85 (m, 3 H), 2.−8-2.57 (m, |
| | | | | 5 H), 2.17 (d, J = 2.8 Hz, 3 H), 2.03 (d, J = 7.6 Hz, 4 H), |
| | | | | 1.95 (s, 5 H), 1.−6-1.72 (m, 2 H), 1.−2-1.42 (m, 1 H) |
| I-541 | BF | AR | 819.1 | 12.18 (d, J = 8.0 Hz, 1 H), 10.85 (s, 1 H), 7.72 (dd, J = |
| | | | | 2.0, 10.8 Hz, 1 H), 7.62 (d, J = 8.0 Hz, 2 H), 7.−6-7.35 |
| | | | | (m, 3 H), 7.−9-6.99 (m, 1 H), 6.87 (s, 1 H), 6.67 (d, J = |
| | | | | 8.0 Hz, 1 H), 6.57 (d, J = 14.4 Hz, 1 H), 6.−3-6.10 (m, |
| | | | | 2 H), 5.11 (d, J = 7.2 Hz, 1 H), 4.37 (t, J = 7.2 Hz, 3 H), |
| | | | | 4.29 (d, J = 15.2 Hz, 2 H), 3.81 (s, 3 H), 3.−9-3.62 (m, |
| | | | | 1 H), 3.58 (t, J = 5.6 Hz, 1 H), 3.−3-3.03 (m, 6 H), |
| | | | | 3.−2-2.92 (m, 3 H), 2.−5-2.77 (m, 3 H), 2.−6-2.63 (m, 2 H), |
| | | | | 2.56 (d, J = 2.4 Hz, 1 H), 2.−5-2.26 (m, 2 H), 2.−7-2.11 |
| | | | | (m, 1 H), 1.−6-1.85 (m, 5 H) |
| I-544 | AL | AK | 790.5 | 12.17 (br d, J = 6.4 Hz, 1 H), 10.77 (s, 1 H), 8.−3-8.08 |
| | | | | (m, 1 H), 7.−1-7.66 (m, 1 H), 7.61 (br d, J = 8.2 Hz, |
| | | | | 2 H), 7.42 (d, J = 8.4 Hz, 2 H), 7.−8-7.00 (m, 1 H), |
| | | | | 6.−3-6.86 (m, 2 H), 6.53 (dd, J = 2.4, 14.9 Hz, 1 H), 6.45 |
| | | | | (dd, J = 2.0, 8.7 Hz, 1 H), 6.16 (br s, 1 H), 5.81 (d, J = |
| | | | | 7.6 Hz, 1 H), 4.−5-4.60 (m, 2 H), 4.−0-4.33 (m, 2 H), |
| | | | | 4.−0-4.24 (m, 1 H), 3.−7-3.59 (m, 2 H), 3.25 (br s, 2 H), |
| | | | | 3.−6-3.06 (m, 6 H), 2.−7-2.68 (m, 4 H), 2.60 (br t, J = |
| | | | | 4.0 Hz, 1 H), 2.−7-2.53 (m, 1 H), 2.−9-2.31 (m, 2 H), |
| | | | | 2.28 (br d, J = 3.2 Hz, 1 H), 2.−3-2.07 (m, 1 H), |
| | | | | 1.−1-1.86 (m, 4 H), 1.−5-1.81 (m, 1 H) |
| I-545 | BG | AK | 731.1 | 12.−0-11.82 (m, 1 H), 10.85 (dd, J = 3.6, 6.8 Hz, 1 H), |
| | | | | 8.−5-8.07 (m, 1 H), 7.−5-7.67 (m, 3 H), 7.59 (d, J = |
| | | | | 8.4 Hz, 2 H), 7.31 (t, J = 8.4 Hz, 1 H), 7.−5-7.05 (m, |
| | | | | 1 H), 6.89 (s, 1 H), 6.72 (d, J = 8.0 Hz, 1 H), 6.−3-6.54 |
| | | | | (m, 2 H), 6.17 (s, 1 H), 4.62 (q, J = 7.2 Hz, 2 H), 4.47 |
| | | | | (t, J = 3.6, 7.6 Hz, I H), 4.−0-4.31 (m, 2 H), 3.−8-3.59 |
| | | | | (m, 2 H), 3.18 (dd, J = 4.4, 11.2 Hz, 3 H), 3.−4-3.08 (m, |
| | | | | 3 H), 3.07 (s, 2 H), 2.−0-2.71 (m, 1 H), 2.61 (d, J = 3.2 |
| | | | | Hz, 1 H), 2.36 (s, 1 H), 2.28 (s, 1 H), 2.−5-2.08 (m, 1 H), |
| | | | | 1.−9-1.87 (m, 1 H) |
| I-550 | DG | DE | 881.2 | 12.−6-11.97 (m, 1 H), 11.−8-10.99 (m, 1 H), 7.72 (dd, |
| | | | | J = 2.0, 10.4 Hz, 1 H), 7.52 (d, J = 8.4 Hz, 2 H), 7.42 |
| | | | | (dd, J = 1.6, 16.8 Hz, 1 H), 7.10 (d, J = 8.4 Hz, 2 H), |
| | | | | 7.−3-6.95 (m, 2 H), 6.84 (d, J = 2.8 Hz, 1 H), 6.73 (dd, J = |
| | | | | 8.0, 12.4 Hz, 2 H), 6.−4-6.17 (m, 1 H), 5.34 (dd, J = |
| | | | | 5.2, 12.8 Hz, 1 H), 4.−8-4.42 (m, 3 H), 4.−9-4.32 (m, |
| | | | | 2 H), 4.09 (s, 2 H), 3.−3-3.84 (m, 1 H), 3.68 (s, 3 H), |
| | | | | 3.61 (s, 3 H), 3.−0-3.25 (m, 4 H), 3.−0-3.02 (m, 1 H), |
| | | | | 2.−7-2.87 (m, 3 H), 2.−2-2.73 (m, 1 H), 2.−2-2.58 (m, |
| | | | | 4 H), 2.55 (s, 1 H), 2.−6-2.30 (m, 2 H), 2.−4-1.98 (m, |
| | | | | 1 H), 1.93 (d, J = 4.8 Hz, 4 H), 1.−1-1.86 (m, 1 H), |
| | | | | 1.−1-1.73 (m, 1 H), 1.−8-1.37 (m, 1 H) |
| I-564 | BJ | AE | 814.2 | 12.−7-12.08 (m, 1 H), 11.−1-10.99 (m, 1 H), |
| | | | | 7.−1-7.77 (m, 1 H), 7.72 (d, J = 7.6 Hz, 1 H), 7.47 (d, J = 8.0 |
| | | | | Hz, 1 H), 7.−4-6.95 (m, 2 H), 6.−2-6.80 (m, 2 H), |
| | | | | 6.−9-6.28 (m, I H), 6.−7-6.07 (m, 1 H), 5.−1-5.32 (m, 1 H), |
| | | | | 4.−0-4.27 (m, 2 H), 3.−3-3.66 (m, 3 H), 3.−2-3.56 (m, |
| | | | | 2 H), 3.−6-2.97 (m, 6 H), 2.−5-2.86 (m, 4 H), 2.−5-2.69 |
| | | | | (m, 1 H), 2.−9-2.63 (m, 2 H), 2.−2-2.58 (m, 1 H), |
| | | | | 2.36 (d, J = 2.8 Hz, 2 H), 2.−8-2.21 (m, 1 H), 2.−9-2.04 |
| | | | | (m, 3 H), 2.01 (d, J = 8.0 Hz, 4 H) |
| I-566 | AD | BK | 788.2 | 12.13 (br s, 1 H), 11.−5-10.79 (m, 1 H), 7.31 (s, 1 H), |
| | | | | 7.−9-7.20 (m, 2 H), 7.−8-6.95 (m, 2 H), 6.83 (br d, J = |
| | | | | 6.0 Hz, 2 H), 6.40 (d, J = 2.8 Hz, 1 H), 6.15 (br s, 1 H), |
| | | | | 5.37 (dd, J = 5.2, 12.8 Hz, 1 H), 4.−9-4.27 (m, 2 H), |
| | | | | 3.71 (s, 3 H), 3.−9-3.61 (m, 2 H), 3.25 (br d, J = 11.2 |
| | | | | Hz, 2 H), 3.−0-3.03 (m, 5 H), 3.−3-2.83 (m, 5 H), |
| | | | | 2.−9-2.60 (m, 3 H), 2.−0-2.22 (m, 2 H), 2.−1-2.13 |
| | | | | (m, 3 H), 2.−5-1.91 (m, 5 H), 1.−8-0.98 (m, 6 H) |
| I-570 | AL | AR | 789.4 | 12.−4-12.14 (m, 1 H), 10.76 (s, 1 H), 7.−7-7.68 (m, |
| | | | | 1 H), 7.64 (d, J = 8.0 Hz, 2 H), 7.−6-7.38 (m, 3 H), |
| | | | | 7.−9-6.99 (m, 1 H), 6.−4-6.86 (m, 2 H), 6.−8-6.50 (m, |
| | | | | 1 H), 6.−9-6.42 (m, 1 H), 6.−3-6.14 (m, 2 H), 5.84 (d, |
| | | | | J = 7.6 Hz, 1 H), 4.−1-4.36 (m, 3 H), 4.−4-4.24 (m, |
| | | | | 2 H), 3.−8-3.57 (m, 2 H), 3.24 (s, 1 H), 3.−3-3.04 (m, |
| | | | | 6 H), 3.−1-2.97 (m, 2 H), 2.−8-2.73 (m, 2 H), |
| | | | | 2.−1-2.65 (m, 2 H), 2.−2-2.59 (m, 1 H), 2.32 (d, J = 1.6 Hz, |
| | | | | 2 H), 2.28 (d, J = 7.2 Hz, 1 H), 2.−3-2.07 (m, 1 H), |
| | | | | 1.−4-1.87 (m, 4 H), 1.−6-1.82 (m, 1 H) |
| I-581 | BQ | AE | 744.4. | 12.15 (br s, 1 H), 11.05 (br s, 1 H), 7.32 (s, 1 H), |
| | | | | 7.−0-7.22 (m, 3 H), 7.19 (br d, J = 8.4 Hz, 1 H), 6.−6-6.77 |
| | | | | (m, 1 H), 6.69 (br d, J = 7.2 Hz, 1 H), 6.40 (br s, 1 H), |
| | | | | 6.−4-6.10 (m, 1 H), 5.70 (br dd, J = 4.8, 12.0 Hz, 1 H), |
| | | | | 4.−3-4.16 (m, 2 H), 3.61 (td, J = 5.6, 10.8 Hz, 2 H), 3.47 |
| | | | | (br d, J = 9.2 Hz, 2 H), 3.−4-2.98 (m, 7 H), 2.86 (br dd, |
| | | | | J = 4.4, 8.8 Hz, 3 H), 2.−7-2.72 (m, 2 H), 2.68 (s, 3 H), |
| | | | | 2.38 (br s, 2 H), 2.−0-2.21 (m, 2 H), 2.18 (br d, J = 6.0 |
| | | | | Hz, 3 H), 2.08 (br d, J = 4.8 Hz, 3 H), 2.01 (br d, J = 4.8 |
| | | | | Hz, 3 H) |
| I-582 | EG | AR | 903.2 | 12.21 (br d, J = 7.6 Hz, I H), 10.86 (s, I H), 7.−6-7.67 |
| | | | | (m, 1 H), 7.−2-7.54 (m, 1 H), 7.−2-7.45 (m, 1 H), |
| | | | | 7.−5-7.35 (m, 2 H), 7.−5-6.93 (m, 1 H), 6.68 (d, J = 8.0 Hz, |
| | | | | 1 H), 6.−1-6.50 (m, 2 H), 6.−3-6.09 (m, 2 H), 5.12 (d, |
| | | | | J = 6.4 Hz, 1 H), 4.−1-4.35 (m, 3 H), 4.−4-4.23 (m, |
| | | | | 2 H), 3.82 (s, 3 H), 3.65 (br t, J = 5.6 Hz, 1 H), 3.58 (br |
| | | | | t, J = 5.6 Hz, 1 H), 3.−2-3.28 (m, 2 H), 3.−2-3.00 (m, |
| | | | | 6 H), 2.−9-2.93 (m, 2 H), 2.−8-2.76 (m, 4 H), |
| | | | | 2.−1-2.56 (m, 1 H), 2.−6-2.26 (m, 2 H), 2.−0-2.10 |
| | | | | (m, 1 H), 1.−8-1.91 (m, 3 H), 1.−1-1.81 (m, 2 H) |
| I-601 | BR | AR | 833.1 | 12.14 (d, J = 6.8 Hz, 1 H), 10.86 (s, 1 H), 7.−4-7.69 (m, |
| | | | | I H), 7.−5-7.36 (m, 1 H), 7.28 (s, 1 H), 7.−5-7.19 (m, |
| | | | | 2 H), 6.−1-6.80 (m, 1 H), 6.68 (d, J = 8.0 Hz, 1 H), 6.58 |
| | | | | (d, J = 14.4 Hz, 1 H), 6.−1-6.37 (m, 1 H), 6.19 (td, J = |
| | | | | 2.0, 13.2 Hz, 1 H), 6.14 (s, 1 H), 5.11 (d, J = 6.8 Hz, 1 H), |
| | | | | 4.36 (q, J = 7.2 Hz, 3 H), 4.−2-4.24 (m, 2 H), 3.82 (s, |
| | | | | 3 H), 3.−7-3.55 (m, 2 H), 3.−0-3.01 (m, 6 H), |
| | | | | 3.−1-2.93 (m, 3 H), 2.−7-2.77 (m, 3 H), 2.−8-2.62 (m, 1 H), |
| | | | | 2.57 (d, J = 3.2 Hz, 1 H), 2.−8-2.22 (m, 3 H), |
| | | | | 2.−8-2.12 (m, 4 H), 1.−6-1.85 (m, 5 H) |
| I-612 | BS | AE | 760.5 | 12.−2-11.93 (m, 1 H), 11.−7-10.92 (m, 1 H), 7.−5-7.24 |
| | | | | (m, 2 H), 7.−2-7.16 (m, 1 H), 7.11 (s, 1 H), 7.04 (br |
| | | | | d, J = 8.0 Hz, 1 H), 6.−8-6.88 (m, 1 H), 6.69 (d, J = 7.2 |
| | | | | Hz, 1 H), 6.51 (d, J = 3.2 Hz, 1 H), 6.−9-6.09 (m, 1 H), |
| | | | | 5.70 (dd, J = 4.8, 11.6 Hz, 1 H), 4.−9-4.30 (m, 2 H), |
| | | | | 3.77 (s, 3 H), 3.−7-3.57 (m, 2 H), 3.49 (br d, J = 9.2 Hz, |
| | | | | 2 H), 3.−4-2.97 (m, 6 H), 2.−2-2.80 (m, 4 H), |
| | | | | 2.−6-2.65 (m, 5 H), 2.38 (br d, J = 2.4 Hz, 1 H), 2.−0-2.17 |
| | | | | (m, 2 H), 2.−2-2.00 (m, 7 H) |
| I-613 | FW | AR | 775.3 | 12.−7-12.08 (m, 1 H), 10.82 (s, 1 H), 7.−4-7.69 (m, |
| | | | | 1 H), 7.−8-7.53 (m, 2 H), 7.−4-7.36 (m, 1 H), 7.12 (d, |
| | | | | J = 8.8 Hz, 2 H), 7.−0-7.04 (m, 2 H), 7.−2-6.99 (m, |
| | | | | 1 H), 6.95 (d, J = 6.0 Hz, 1 H), 6.86 (br s, 1 H), |
| | | | | 6.−2-6.17 (m, 1 H), 6.14 (br d, J = 3.6 Hz, 1 H), 4.−0-4.35 |
| | | | | (m, 3 H), 4.−4-4.28 (m, 1 H), 3.83 (dd, J = 4.8, 11.6 Hz, |
| | | | | 1 H), 3.−7-3.56 (m, 2 H), 3.37 (br d, J = 5.6 Hz, 4 H), |
| | | | | 3.19 (br d, J = 4.4 Hz, 6 H), 3.−4-3.03 (m, 3 H), |
| | | | | 3.−2-2.93 (m, 3 H), 2.−2-2.61 (m, 1 H), 2.−6-2.52 (m, 1 H), |
| | | | | 2.−5-2.25 (m, 2 H), 2.−5-2.16 (m, 1 H), 2.−6-1.98 (m, |
| | | | | 1 H) |
| I-614 | DO | DE | 895.2 | 12.07 (s, 1 H), 11.09 (s, I H), 7.71 (dd, J = 2.0, 10.4 Hz, |
| | | | | 1 H), 7.52 (d, J = 8.8 Hz, 2 H), 7.41 (dd, J = 1.2, 16.8 |
| | | | | Hz, 1 H), 7.−2-7.02 (m, 4 H), 7.−2-6.96 (m, 2 H), 6.83 |
| | | | | (s, 1 H), 6.19 (td, J = 2.0, 11.2 Hz, 1 H), 5.37 (dd, J = |
| | | | | 5.6, 12.8 Hz, 1 H), 4.47 (s, 3 H), 4.35 (q, J = 7.2 Hz, 2 H), |
| | | | | 4.08 (s, 2 H), 3.88 (d, J = 13.2 Hz, 1 H), 3.−3-3.72 (m, |
| | | | | 1 H), 3.−3-3.52 (m, 3 H), 3.23 (s, 4 H), 3.−9-3.01 (m, |
| | | | | 1 H), 2.−8-2.88 (m, 3 H), 2.−7-2.75 (m, 2 H), |
| | | | | 2.−5-2.68 (m, 1 H), 2.−7-2.62 (m, 1 H), 2.60 (s, 1 H), 2.51 |
| | | | | (s, 4 H), 2.−8-2.41 (m, 2 H), 2.−6-2.29 (m, 2 H), |
| | | | | 2.−1-1.96 (m, 3 H), 1.−5-1.83 (m, 2 H), 1.−1-1.69 |
| | | | | (m, 1 H), 1.−9-1.36 (m, 1 H) |
| I-615 | DQ | DE | 895.4 | 12.06 (br s, 1 H), 11.−1-11.04 (m, 1 H), 7.71 (dd, J = |
| | | | | 2.0, 10.8 Hz, 1 H), 7.50 (d, J = 8.8 Hz, 2 H), 7.41 |
| | | | | (dd, J = 1.2, 16.8 Hz, 1 H), 7.06 (br d, J = 8.4 Hz, 2 H), |
| | | | | 7.−1-6.93 (m, 2 H), 6.83 (d, J = 2.4 Hz, 1 H), 6.73 (d, J = 8.0 |
| | | | | Hz, 1 H), 6.68 (d, J = 8.0 Hz, 1 H), 6.20 (td, J = 2.0, 11.2 |
| | | | | Hz, 1 H), 5.32 (br dd, J = 5.2, 12.8 Hz, 1 H), 4.47 (br d, |
| | | | | J = 6.0 Hz, 3 H), 4.35 (q, J = 7.2 Hz, 2 H), 4.08 (br d, J = |
| | | | | 6.4 Hz, 2 H), 3.−7-3.90 (m, 2 H), 3.−9-3.76 (m, 3 H), |
| | | | | 3.−4-3.60 (m, 2 H), 3.59 (s, 3 H), 3.−0-2.99 (m, 1 H), |
| | | | | 2.−8-2.82 (m, 4 H), 2.−8-2.69 (m, 3 H), 2.−8-2.61 (m, |
| | | | | 2 H), 2.−4-2.27 (m, 2 H), 2.−3-1.96 (m, 1 H), |
| | | | | 1.−4-1.86 (m, 2 H), 1.−3-1.72 (m, 3 H), 1.−1-1.63 (m, 1 H), |
| | | | | 1.−4-1.38 (m, 1 H), 1.−2-1.20 (m, 3 H) |
| I-626^{c} | DT | AR | 875.5 | 12.−8-11.86 (m, 1 H), 10.85 (s, 1 H), 8.58 (d, J = 8.4 |
| | | | | Hz, 1 H), 7.−7-7.66 (m, 3 H), 7.53 (d, J = 8.4 Hz, 2 H), |
| | | | | 7.−7-7.28 (m, 1 H), 7.09 (d, J = 8.0 Hz, 2 H), |
| | | | | 7.−3-6.79 (m, 2 H), 6.63 (d, J = 8.4 Hz, 1 H), 6.−8-6.01 (m, |
| | | | | 2 H), 4.−3-4.65 (m, 1 H), 4.−1-4.28 (m, 4 H), 3.95 (s, |
| | | | | 4 H), 3.59 (s, 2 H), 3.26 (s, 4 H), 3.−4-3.04 (m, 6 H), |
| | | | | 2.98 (d, J = 8.8 Hz, 3 H), 2.79 (s, 1 H), 2.34 (s, 2 H), |
| | | | | 2.−4-2.04 (m, 1 H), 1.91 (s, 5 H) |
| I-628 | BT | AR | 774.2 | 12.−2-12.00 (m, 1 H), 10.82 (s, 1 H), 7.−8-7.67 (m, |
| | | | | 1 H), 7.−6-7.57 (m, 2 H), 7.−7-7.36 (m, 3 H), |
| | | | | 7.−2-6.94 (m, 4 H), 6.87 (s, 1 H), 6.−8-6.09 (m, 2 H), |
| | | | | 4.−5-4.34 (m, 3 H), 4.−3-4.25 (m, 1 H), 3.−7-3.76 (m, 1 H), |
| | | | | 3.−1-3.57 (m, 2 H), 3.−4-3.45 (m, 2 H), 3.−2-3.03 (m, |
| | | | | 6 H), 3.−1-2.95 (m, 2 H), 2.−7-2.78 (m, 2 H), |
| | | | | 2.−3-2.60 (m, 2 H), 2.−8-2.30 (m, 2 H), 2.−0-2.20 |
| | | | | (m, 2 H), 2.−6-1.98 (m, 1 H), 1.−7-1.85 (m, 4 H) |
| I-630 | BV | AW | 796.3 | 11.−9-11.94 (m, I H), 11.10 ( s, 1 H), 7.15 (d, J = 10.4 |
| | | | | Hz, 1 H), 7.10 (d, J = 6.4 Hz, 1 H), 7.−3-7.00 (m, 2 H), |
| | | | | 6.97 (s, 1 H), 6.91 (d, J = 5.6 Hz, 1 H), 6.50 (s, 1 H), |
| | | | | 5.−0-5.34 (m, 1 H), 4.−1-4.44 (m, 1 H), 3.−8-3.83 (m, |
| | | | | 1 H), 3.76 (s, 3 H), 3.72 (s, 3 H), 3.26 (s, 3 H), 3.20 (s, |
| | | | | 2 H), 3.11 (d, J = 10.4 Hz, 2 H), 3.−7-3.03 (m, 2 H), |
| | | | | 3.−2-2.98 (m, 2 H), 2.95 (s, 1 H), 2.−1-2.86 (m, 2 H), 2.72 |
| | | | | (t, J = 13.6 Hz, 2 H), 2.−5-2.57 (m, 2 H), 2.−1-2.36 |
| | | | | (m, 1 H), 2.−3-2.06 (m, 2 H), 2.04 (d, J = 9.2 Hz, 3 H), |
| | | | | 1.95 (d, J = 1.2 Hz, 1 H), 1.93 (s, 1 H), 1.−6-1.74 (m, |
| | | | | 2 H), 1.−1-1.43 (m, 1 H) |
| I-631 | BX | AE | 778.1 | 12.20 (s, 1 H), 11.−4-11.02 (m, 1 H), 7.40 (d, J = 8.0 |
| | | | | Hz, 1 H), 7.12 (d, J = 11.2 Hz, 1 H), 7.−6-6.97 (m, 2 H), |
| | | | | 6.−6-6.84 (m, 2 H), 6.44 (s, 1 H), 6.−0-6.13 (m, 1 H), |
| | | | | 5.−4-5.31 (m, 1 H), 4.−9-4.31 (m, 2 H), 3.70 (s, 3 H), |
| | | | | 3.−6-3.58 (m, 2 H), 3.28 (d, J = 11.0 Hz, 3 H), 3.−9- |
| | | | | 3.00 (m, 6 H), 2.−5-2.86 (m, 3 H), 2.−6-2.62 (m, 2 H), |
| | | | | 2.36 (d, J = 2.8 Hz, 1 H), 2.28 (s, 1 H), 2.−8-2.12 (m, |
| | | | | 3 H), 2.08 (d, J = 4.4 Hz, 3 H), 2.−7-1.97 (m, 3 H), |
| | | | | 1.−7-1.90 (m, 2 H) |
| I-632 | Z | AH | 744.3 | 12.14 (s, I H), 10.89 (s, 1 H), 7.40 (d, J = 7.6 Hz, 1 H), |
| | | | | 7.−5-7.22 (m, 3 H), 7.−2-7.02 (m, 2 H), 6.−3-6.79 (m, |
| | | | | 1 H), 6.39 (s, 1 H), 6.−0-6.11 (m, 1 H), 4.−7-4.30 (m, |
| | | | | 6 H), 3.61 (td, J = 5.6, 10.8 Hz, 2 H), 3.35 (s, 2 H), |
| | | | | 3.−1-2.96 (m, 6 H), 2.88 (s, 2 H), 2.76 (d, J = 6.4 Hz, 1 H), |
| | | | | 2.−8-2.61 (m, 2 H), 2.−2-2.21 (m, 4 H), 2.18 (d, J = |
| | | | | 6.0 Hz, 3 H), 2.08 (d, J = 4.8 Hz, 3 H), 2.01 (s, 4 H) |
| I-633 | AX | BY | 793.4 | 11.10 (s, 1 H), 7.−1-7.22 (m, 3 H), 7.13 (s, 1 H), 7.06 (d, |
| | | | | J = 7.2 Hz, 1 H), 7.00 (s, 3 H), 6.−2-6.19 (m, 1 H), 5.37 |
| | | | | (dd, J = 5.2, 12.4 Hz, 1 H), 4.36 (s, 2 H), 3.74 (d, J = 19.6 |
| | | | | Hz, 6 H), 3.62 (d, J = 6.4 Hz, 2 H), 3.26 (s, 1 H), |
| | | | | 3.−2-2.99 (m, 6 H), 2.91 (s, 3 H), 2.63 (d, J = 18.4 Hz, 4 H), |
| | | | | 2.−0-2.26 (m, 2 H), 2.09 (d, J = 6.4 Hz, 3 H), 2.00 (s, |
| | | | | 5 H) |
| I-638 | BF | DY | 836.1 | 10.85 (s, 1 H), 7.−5-7.64 (m, 2 H), 7.58 (d, J = 7.6 Hz, |
| | | | | 2 H), 7.−0-7.41 (m, 3 H), 7.−0-7.35 (m, 1 H), 6.67 (d, |
| | | | | J = 8.0 Hz, 1 H), 6.57 (d, J = 14.4 Hz, 1 H), 6.29 (s, 1 H), |
| | | | | 6.19 (d, J = 14.4 Hz, 1 H), 5.11 (d, J = 6.4 Hz, 1 H), 4.36 |
| | | | | (dd, J = 7.2, 15.2 Hz, 4 H), 4.−0-4.23 (m, 1 H), 3.81 (s, |
| | | | | 3 H), 3.−8-3.62 (m, 1 H), 3.59 (s, 1 H), 3.−0-3.28 (m, |
| | | | | 1 H), 3.−6-3.02 (m, 6 H), 3.−2-2.93 (m, 3 H), |
| | | | | 2.−6-2.77 (m, 3 H), 2.−3-2.66 (m, 1 H), 2.57 (s, 1 H), |
| | | | | 2.35 (s, 1 H), 2.28 (s, 1 H), 2.−8-2.10 (m, 1 H), 1.91 (s, 5 H) |
| I-639 | EA | AE | 843.2 | 12.−6-11.92 (m, 1 H), 11.−4-10.77 (m, 1 H), |
| | | | | 7.−4-7.25 (m, 3 H), 7.−2-6.93 (m, 3 H), 6.−6-6.76 (m, 1 H), |
| | | | | 6.34 (d, J = 3.2 Hz, 1 H), 6.−5-6.08 (m, 1 H), |
| | | | | 5.−5-5.30 (m, 1 H), 4.31 (s, 2 H), 3.62 (s, 3 H), 3.−1-3.55 |
| | | | | (m, 2 H), 3.−1-3.07 (m, 6 H), 3.−6-3.00 (m, 2 H), |
| | | | | 3.−0-2.82 (m, 2 H), 2.−9-2.72 (m, 1 H), 2.68 (d, J = 3.6 Hz, |
| | | | | 6 H), 2.−6-2.58 (m, 3 H), 2.−1-2.31 (m, 2 H), 2.26 (s, |
| | | | | 1 H), 2.−0-2.05 (m, 3 H), 2.−4-1.98 (m, 1 H) |
| I-650 | EJ | AR | 819.3 | 12.−0-12.04 (m, 1 H), 10.−5-10.76 (m, 1 H), |
| | | | | 7.−4-7.69 (m, 1 H), 7.53 (br d, J = 7.9 Hz, 2 H), 7.−5-7.36 |
| | | | | (m, 1 H), 7.10 (br d, J = 8.3 Hz, 2 H), 6.96 (br dd, J = |
| | | | | 5.7, 17.7 Hz, I H), 6.−9-6.84 (m, 1 H), 6.75 (br d, J = |
| | | | | 7.0 Hz, 1 H), 6.51 (br d, J = 12.8 Hz, 1 H), 6.−3-6.11 |
| | | | | (m, 2 H), 5.29 (br d, J = 6.9 Hz, 1 H), 4.−9-4.34 (m, |
| | | | | 3 H), 4.−4-4.26 (m, 2 H), 3.89 (br d, J = 11.4 Hz, 2 H), |
| | | | | 3.80 (s, 3 H), 3.−9-3.62 (m, 1 H), 3.58 (br s, 1 H), |
| | | | | 3.−1-3.04 (m, 6 H), 2.97 (br d, J = 7.9 Hz, 2 H), 2.−8-2.77 |
| | | | | (m, 4 H), 2.−9-2.65 (m, 1 H), 2.33 (br s, 2 H), 2.27 (br |
| | | | | dd, J = 1.1, 2.8 Hz, 1 H), 2.−7-2.10 (m, 1 H), 1.−8-1.87 |
| | | | | (m, 2 H), 1.82 (br s, 2 H) |
| I-668 | Z | EM | 760.3 | 12.02 (s, 1 H), 10.89 (s, 1 H), 7.−4-7.25 (m, 2 H), |
| | | | | 7.−4-6.99 (m, 4 H), 6.−7-6.87 (m, 1 H), 6.49 (s, 1 H), |
| | | | | 6.−8-6.06 (m, 1 H), 4.−9-4.28 (m, 6 H), 3.76 (s, 3 H), |
| | | | | 3.−5-3.57 (m, 2 H), 3.38 (d, J = 10.0 Hz, 2 H), 3.−6-2.96 |
| | | | | (m, 6 H), 2.−3-2.77 (m, 3 H), 2.−2-2.60 (m, 2 H), |
| | | | | 2.−9-2.24 (m, 3 H), 2.18 (dd, J = 5.2, 12.4 Hz, 1 H), |
| | | | | 2.−1-1.99 (m, 7 H) |
| I-807 | AD | BD | 770.1 | 11.71 (s, 1 H), 11.29-10.93 (m, 1 H), 8.15-8.08 (m, |
| | | | | 1 H), 7.70 (s, 1 H), 7.41 (s, 1 H), 7.29 (s, 1 H), 7.23 (s, |
| | | | | 2 H), 7.04-6.97 (m, 2 H), 6.90 (d, J = 7.2 Hz, 1 H), |
| | | | | 6.86-6.74 (m, 1 H), 6.18-6.08 (m, 2 H), 5.41-5.34 (m, 1 H), |
| | | | | 4.63 (q, J = 7.2 Hz, 2 H), 4.40-4.32 (m, 2 H), 3.71 (s, |
| | | | | 3 H), 3.65 (t, J = 6.0 Hz, 1 H), 3.60 (t, J = 6.0 Hz, 1 H), |
| | | | | 3.26 (d, J = 11.6 Hz, 3 H), 3.11 (t, J = 6.8 Hz, 2 H), |
| | | | | 2.94-2.85 (m, 3 H), 2.73-2.66 (m, 2 H), 2.61 (s, 1 H), 2.34 |
| | | | | (d, J = 2.0 Hz, 2 H), 2.27 (d, J = 1.2 Hz, 1 H), 2.18-2.14 |
| | | | | (m, 3 H), 1.96 (s, 3 H) |
| I-819 | EQ | AR | 810.2 | 12.26-12.12 (m, 1 H), 11.13-10.92 (m, 1 H), |
| | | | | 8.51-8.43 (m, 1 H), 7.76-7.70 (m, 1 H), 7.65 (d, J = 8.0 Hz, |
| | | | | 2 H), 7.48 (d, J = 8.0 Hz, 2 H), 7.45-7.38 (m, 1 H), |
| | | | | 7.30-7.24 (m, 1 H), 7.22-7.17 (m, 1 H), 7.10-7.01 (m, 1 H), |
| | | | | 6.89 (s, 1 H), 6.74-6.66 (m, 1 H), 6.24-6.14 (m, 2 H), |
| | | | | 5.74-5.65 (m, 1 H), 4.42-4.31 (m, 4 H), 3.68-3.57 (m, |
| | | | | 2 H), 3.48 (d, J = 8.8 Hz, 2 H), 3.26-3.15 (m, 3 H), |
| | | | | 3.13-3.05 (m, 2 H), 3.02-2.97 (m, 2 H), 2.91-2.80 (m, 4 H), |
| | | | | 2.73 (d, J = 3.2 Hz, 1 H), 2.69 (s, 4 H), 2.40-2.17 (m, |
| | | | | 4 H), 2.06-1.96 (m, 4 H) |
| I-825 | EC | AR | 819.6 | 12.10-11.90 (m, 1 H), 10.79 (s, 1 H), 7.84-7.63 (m, |
| | | | | 1 H), 7.51-7.34 (m, 1 H), 7.32-7.24 (m, 1 H), |
| | | | | 7.10-7.05 (m, 1 H), 7.01-6.87 (m, 3 H), 6.59-6.43 (m, 3 H), |
| | | | | 6.25-6.08 (m, 2 H), 5.83 (d, J = 7.6 Hz, 1 H), |
| | | | | 4.40-4.26 (m, 5 H), 3.75 (d,.J = 4.0 Hz, 3 H), 3.68-3.56 (m, |
| | | | | 2 H), 3.26-3.03 (m, 7 H), 3.00-2.90 (m, 2 H), 2.83- |
| | | | | 2.62 (m, 5 H), 2.42-2.21 (m, 3 H), 2.14-2.07 (m, 1 H), |
| | | | | 1.96-1.84 (m, 5 H) |
| I-826 | AX | BK | 804.3 | 12.09 (br s, 1 H), 11.15 (br d, J = 5.2 Hz, 1 H), 7.37 (br |
| | | | | d, J = 8.0 Hz, 1 H), 7.15 (s, 1 H), 7.11-7.04 (m, 3 H), |
| | | | | 7.00-6.94 (m, 2 H), 6.56 (br s, 1 H), 6.18 (br s, 1 H), |
| | | | | 5.43 (br dd, J = 5.2, 12.4 Hz, 1 H), 4.47-4.40 (m, 2 H), |
| | | | | 3.83 (s, 3 H), 3.77 (s, 3 H), 3.73 (br d, J = 6.4 Hz, 2 H), |
| | | | | 3.49-3.41 (m, 1 H), 3.33 (br d, J = 11.2 Hz, 2 H), 3.29-3.09 |
| | | | | (m, 6 H), 3.00-2.94 (m, 3 H), 2.86-2.81 (m, 1 H), |
| | | | | 2.80-2.70 (m, 2 H), 2.67 (br s, 1 H), 2.44 (br s, 1 H), |
| | | | | 2.32 (br s, 1 H), 2.05 (br s, 4 H), 1.12-1.06 (m, 6 H) |
| I-731 | EU | AR | 838.2 | 12.13 (d, J = 8.8 Hz, 1 H), 10.85 (s, 1 H), 7.77-7.66 (m, |
| | | | | 1 H), 7.49-7.29 (m, 2 H), 7.02-6.87 (m, 3 H), |
| | | | | 6.71-6.55 (m, 3 H), 6.26-6.03 (m, 2 H), 5.14 (d, J = 6.4 Hz, |
| | | | | 1 H), 4.39-4.25 (m, 5 H), 3.82 (s, 3 H), 3.67-3.55 (m, |
| | | | | 2 H), 3.39 (s, 4 H), 3.16 (s, 3 H), 3.07 (s, 6 H), 3.02-2.92 |
| | | | | (m, 3 H), 2.86-2.76 (m, 1 H), 2.57 (s, 1 H), 2.36-2.24 |
| | | | | (m, 2 H), 2.19-2.09 (m, 1 H), 1.93 (dq, J = 4.4, 12.8 Hz, |
| | | | | 1 H) |
| I-835 | GB | AR | 858.3 | 12.13 (s, 1 H), 10.82 (s, 1 H), 7.81-7.65 (m, 1 H), 7.53 |
| | | | | (d, J = 7.6 Hz, 2 H), 7.44 (s, 1 H), 7.14-7.00 (m, 4 H), |
| | | | | 6.96 (d, J = 7.6 Hz, 2 H), 6.86 (s, 1 H), 6.26-6.05 (m, |
| | | | | 2 H), 4.41-4.29 (m, 4 H), 3.81 (dd, J = 4.4, 12.0 Hz, |
| | | | | 1 H), 3.69-3.57 (m, 2 H), 3.41 (s, 2 H), 3.22 (s, 3 H), |
| | | | | 3.21-3.03 (m, 6 H), 3.01-2.95 (m, 2 H), 2.71 (s, 6 H), |
| | | | | 2.67-2.59 (m, 2 H), 2.44-2.39 (m, 1 H), 2.38-2.12 (m, |
| | | | | 4 H), 2.04-1.89 (m, 3 H), 1.69-1.57 (m, 2 H) |
| I-676^{c} | EX | AR | 856.3 | 12.20 (br s, 1 H), 10.85 (s, 1 H), 7.75-7.68 (m, 1 H), |
| | | | | 7.47-7.34 (m, 2 H), 7.10-6.96 (m, 2 H), 6.70 (d, J = |
| | | | | 8.0 Hz, 1 H), 6.65 (br s, 1 H), 6.59 (d, J = 14.4 Hz, 1 H), |
| | | | | 6.22-6.12 (m, 2 H), 5.13 (d, J = 6.4 Hz, 1 H), |
| | | | | 4.39-4.34 (m, 3 H), 4.33-4.23 (m, 2 H), 3.82 (s, 3 H), |
| | | | | 3.66-3.56 (m, 2 H), 3.26 (br s, 4 H), 3.21-3.02 (m, 10 H), |
| | | | | 3.00-2.96 (m, 2 H), 2.86-2.76 (m, 1 H), 2.58-2.52 (m, 1 H), |
| | | | | 2.35-2.24 (m, 2 H), 2.19-2.10 (m, 1 H), 1.93 |
| | | | | (br dd, J = 4.4, 12.8 Hz, 1 H) |
| I-859 | Z | EY | 724.1 | 11.95 (s, 1 H), 10.8 (s, 1 H), 7.33-7.27 (m, 1 H), |
| | | | | 7.10-7.03 (m, 3 H), 6.99 (d, J = 7.6 Hz, 2 H), 6.96-6.88 |
| | | | | (m, 1 H), 6.56-6.41 (m, 1 H), 6.19-6.10 (m, 1 H), |
| | | | | 4.37-4.29 (m, 2 H), 3.85-3.80 (m, 1 H), 3.76 (s, 3 H), 3.62 |
| | | | | (d, J = 5.6 Hz, 2 H), 3.51 (d, J = 10.4 Hz, 2 H), 3.22-2.93 |
| | | | | (m, 6 H), 2.83-2.75 (m, 2 H), 2.73-2.67 (m, 1 H), |
| | | | | 2.47-2.42 (m, 1 H), 2.40-2.33 (m, 2 H), 2.30-2.16 (m, 2 H), |
| | | | | 2.08 (d, J = 7.2 Hz, 3 H), 2.03 (d, J = 4.4 Hz, 1 H), 1.95 |
| | | | | (s, 4 H) |
| I-875 | FA | BK | 858.2 | 12.16-12.07 (m, 1 H), 11.08 (s, 1 H), 7.56-7.49 (m, |
| | | | | 2 H), 7.06 (d, J = 8.4 Hz, 2 H), 7.00-6.90 (m, 3 H), |
| | | | | 6.90-6.84 (m, 2 H), 6.18-6.09 (m, 1 H), 5.42-5.29 (m, 1 H), |
| | | | | 4.44-4.33 (m, 2 H), 3.70-3.62 (m, 5 H), 3.23 (s, 6 H), |
| | | | | 3.20-3.16 (m, 3 H), 3.11-3.04 (m, 2 H), 3.01-2.84 (m, |
| | | | | 3 H), 2.72 (s, 6 H), 2.67-2.59 (m, 2 H), 2.42-2.32 (m, |
| | | | | 3 H), 2.26 (d, J = 2.8 Hz, 1 H), 2.00-1.94 (m, 2 H), |
| | | | | 1.74-1.63 (m, 2 H), 1.07-0.99 (m, 6 H) |
| I-876 | FC | BK | 855.1 | 12.27-12.08 (m, 1 H), 11.18-11.01 (m, 1 H), 7.31 (d, |
| | | | | J = 13.2 Hz, 1 H), 7.17 (d, J = 8.0 Hz, 1 H), 7.00-6.90 |
| | | | | (m, 4 H), 6.51 (d, J = 2.0 Hz, 1 H), 6.19-6.09 (m, 1 H), |
| | | | | 5.42-5.33 (m, 1 H), 4.36 (s, 2 H), 3.66 (d, J = 5.6 Hz, |
| | | | | 2 H), 3.61 (s, 3 H), 3.15 (s, 8 H), 3.07-3.00 (m, 2 H), |
| | | | | 3.00-2.83 (m, 3 H), 2.81-2.53 (m, 9 H), 2.38-2.22 (m, |
| | | | | 2 H), 2.04-1.96 (m, 1 H), 1.04 (d, J = 6.8 Hz, 6 H) |
| I-894 | GG | GE | 804.5 | 12.24-12.12 (m, 1 H), 10.78 (s, 1 H), 8.09-8.04 (m, |
| | | | | 1 H), 7.72-7.65 (m, 1 H), 7.65-7.60 (m, 2 H), 7.43 (d, |
| | | | | J = 7.6 Hz, 2 H), 7.05-6.99 (m, 1 H), 6.93-6.86 (m, |
| | | | | 2 H), 6.56-6.50 (m, 1 H), 6.45 (d, J = 8.8 Hz, 1 H), 6.14 |
| | | | | (s, 1 H), 5.81 (d, J = 7.6 Hz, 1 H), 4.64-4.55 (m, 1 H), |
| | | | | 4.47-4.32 (m, 3 H), 4.30-4.25 (m, 1 H), 3.70-3.54 (m, |
| | | | | 3 H), 3.25 (s, 1 H), 3.21-3.00 (m, 6 H), 2.83-2.63 (m, |
| | | | | 5 H), 2.61-2.55 (m, 1 H), 2.34-2.23 (m, 2 H), 2.13-2.07 |
| | | | | (m, 1 H), 1.92-1.82 (m, 5 H), 1.08-1.00 (m, 3 H) |
| I-895 | GH | GE | 804.2 | 12.26-12.12 (m, 1 H), 10.78 (s, 1 H), 8.15-8.00 (m, |
| | | | | 1 H), 7.73-7.59 (m, 3 H), 7.43 (d, J = 8.0 Hz, 2 H), |
| | | | | 7.09-6.97 (m, 1 H), 6.95-6.84 (m, 2 H), 6.57-6.50 (m, 1 H), |
| | | | | 6.49-6.41 (m, 1 H), 6.15 (s, 1 H), 5.82 (d, J = 7.6 Hz, |
| | | | | 1 H), 4.68-4.53 (m, 1 H), 4.48-4.34 (m, 3 H), 4.31-4.25 |
| | | | | (m, 1 H), 3.73-3.54 (m, 3 H), 3.26 (d, J = 2.0 Hz, |
| | | | | 1 H), 3.20-2.97 (m, 6 H), 2.86-2.64 (m, 5 H), |
| | | | | 2.63-2.55 (m, 1 H), 2.35-2.22 (m, 2 H), 2.15-2.05 |
| | | | | (m, 1 H), 1.94-1.84 (m, 5 H), 1.10-1.00 (m, 3 H) |
| I-802 | GJ | AR | 903.3 | 12.25-11.99 (m, 1 H), 10.96-10.76 (m, 1 H), |
| | | | | 7.78-7.63 (m, 1 H), 7.59-7.46 (m, 2 H), 7.45-7.37 (m, 1 H), |
| | | | | 7.11-6.98 (m, 2 H), 6.94-6.83 (m, 2 H), 6.66-6.48 (m, |
| | | | | 2 H), 6.26-6.04 (m, 2 H), 5.16-4.93 (m, 1 H), 4.40- |
| | | | | 4.34 (m, 3 H), 4.31-4.23 (m, 2 H), 3.80-3.77 (m, 2 H), |
| | | | | 3.67-3.56 (m, 2 H), 3.25-3.18 (m, 8 H), 3.17-3.03 (m, |
| | | | | 6 H), 3.00-2.94 (m, 2 H), 2.75-2.65 (m, 6 H), 2.42-2.31 |
| | | | | (m, 4 H), 2.17-2.10 (m, 1 H), 1.92-1.85 (m, 2 H), |
| | | | | 1.71-1.54 (m, 2 H), 1.11-0.95 (m, 1 H) |
| I-904 | FE | AR | 872.0 | 1.90-2.02 (m, 1 H), 2.08-2.17 (m, 1 H), 2.24-2.42 |
| | | | | (m, 3 H), 2.80 (ddd, J = 17.6, 12.8, 4.8 Hz, 1 H), |
| | | | | 2.93-3.01 (m, 3 H), 3.01-3.19 (m, 6 H), 3.24 (br s, 4 H), |
| | | | | 3.33 (br s, 5 H), 3.83 (s, 3 H), 4.21-4.45 (m, 6 H), |
| | | | | 6.08-6.25 (m, 2 H), 6.51 (br s, 1 H), 6.64 (br d, J = 14.4 Hz, |
| | | | | 1 H), 6.84 (br d, J = 6.4 Hz, 1 H), 6.90-7.01 (m, 1 H), |
| | | | | 7.25 (br d, J = 8.4 Hz, 1 H), 7.30-7.46 (m, 2 H), |
| | | | | 7.66-7.76 (m, 1 H), 10.86 (s, 1 H), 12.15-12.24 (m, 1 H) |
| I-906 | FH | AR | 838.2 | 12.44-11.98 (m, 1 H), 10.86 (br d, J = 2.4 Hz, 1 H), |
| | | | | 7.76-7.70 (m, 1 H), 7.51-7.35 (m, 3 H), 7.21 (t, J = 8.8 |
| | | | | Hz, 1 H), 7.09-6.99 (m, 1 H), 6.90 (d,.J = 2.8 Hz, 1 H), |
| | | | | 6.71 (d, J = 8.0 Hz, 1 H), 6.59 (d, J = 14.4 Hz, 1 H), |
| | | | | 6.24-6.15 (m, 2 H), 5.14 (d, J = 6.8 Hz, 1 H), 4.40-4.36 |
| | | | | (m, 3 H), 4.35-4.25 (m, 2 H), 3.83 (s, 3 H), 3.66 (br t, J = |
| | | | | 5.6 Hz, 1 H), 3.59 (br t, J = 5.6 Hz, 1 H), 3.30 (s, 2 H), |
| | | | | 3.24 (br d, J = 4.4 Hz, 5 H), 3.12 (br s, 6 H), 3.01-2.97 |
| | | | | (m, 2 H), 2.87-2.77 (m, 1 H), 2.57 (br d, J = 2.8 Hz, |
| | | | | 1 H), 2.38-2.24 (m, 3 H), 2.19-2.11 (m, I H), |
| | | | | 2.00-1.88 (m, 1 H) (m, 5 H), 2.00-1.90 (m, 1 H) |
| I-677 | FK | AR | 856.3 | 12.27-12.19 (m, 1 H), 10.86 (s, 1 H), 7.74-7.68 (m, |
| | | | | 1 H), 7.45-7.36 (m, 1 H), 7.35-7.27 (m, 1 H), |
| | | | | 7.06-6.97 (m, 2 H), 6.71 (d, J = 8.0 Hz, 1 H), 6.67 (br s, 1 H), |
| | | | | 6.59 (d, J = 14.4 Hz, 1 H), 6.22-6.12 (m, 2 H), 5.14 (br |
| | | | | d, J = 7.2 Hz, 1 H), 4.39-4.34 (m, 3 H), 4.32-4.24 (m, |
| | | | | 2 H), 3.82 (s, 3 H), 3.66-3.56 (m, 2 H), 3.28 (br s, 6 H), |
| | | | | 3.12 (br s, 6 H), 3.02-2.93 (m, 3 H), 2.85-2.76 (m, 1 H), |
| | | | | 2.56 (br d, J = 2.4 Hz, I H), 2.33 (br s, 2 H), 2.27 (br dd, |
| | | | | J = 2.4, 4.0 Hz, 1 H), 2.17-2.10 (m, 1 H), 1.99-1.88 |
| | | | | (m, 1 H) |
| I-800 | FL | AR | 788.4 | 12.19-12.01 (m, 1 H), 10.86-10.79 (m, 1 H), 7.76 (br |
| | | | | d, J = 3.6 Hz, 1 H), 7.44-7.33 (m, 1 H), 7.30-7.26 (m, |
| | | | | 1 H), 7.25-7.18 (m, 2 H), 7.09-7.02 (m, 2 H), |
| | | | | 7.00-6.96 (m, 1 H), 6.90-6.79 (m, 1 H), 6.41-6.34 (m, 1 H), |
| | | | | 6.23-6.09 (m, 2 H), 4.39-4.28 (m, 4 H), 3.86-3.77 (m, |
| | | | | 1 H), 3.65-3.54 (m, 2 H), 3.52-3.46 (m, 2 H), |
| | | | | 3.23-3.02 (m, 6 H), 3.01-2.93 (m, 3 H), 2.84-2.77 (m, 2 H), |
| | | | | 2.72-2.62 (m, 2 H), 2.34-2.30 (m, 1 H), 2.28-2.17 (m, |
| | | | | 2 H), 2.17-2.13 (m, 3 H), 2.06-1.99 (m, 1 H), |
| | | | | 1.95-1.83 (m, 4 H) |
| I-801 | FO | AR | 792.3 | 12.24-12.16 (m, 1 H), 10.82 (s, 1 H), 7.75-7.67 (m, |
| | | | | 1 H), 7.53 (t, J = 3.2, 8.0 Hz, 1 H), 7.43 (d, J = 1.2 Hz, |
| | | | | 1 H), 7.33-7.26 (m, 2 H), 7.08-7.06 (m, 1 H), 7.03 (d, |
| | | | | J = 2.8 Hz, 1 H), 7.03-6.96 (m, 2 H), 6.61 (s, 1 H), |
| | | | | 6.22-6.11 (m, 2 H), 4.39-4.35 (m, 3 H), 4.34-4.28 (m, 1 H), |
| | | | | 3.82 (dd, J = 4.8, 11.7 Hz, 1 H), 3.64 (t, J = 5.2 Hz, 1 H), |
| | | | | 3.58 (t, J = 5.6 Hz, 1 H), 3.49 (d, J = 11.2 Hz, 2 H), |
| | | | | 3.27-3.02 (m, 6 H), 3.02-2.89 (m, 3 H), 2.79 (q, J = 10.8 |
| | | | | Hz, 3 H), 2.70-2.62 (m, 1 H), 2.33 (s, 1 H), 2.29-2.15 |
| | | | | (m, 2 H), 2.04-1.86 (m, 5 H) |
| I-927 | AD | FS | 854.4 | 12.15 (br s, 1 H), 11.29-10.86 (m, 1 H), 7.73-7.60 (m, |
| | | | | 1 H), 7.48-7.19 (m, 4 H), 7.06-6.96 (m, 2 H), |
| | | | | 6.93-6.77 (m, 2 H), 6.40 (d, J = 2.8 Hz, 1 H), 6.24-6.08 (m, |
| | | | | 2 H), 5.38 (br dd, J = 5.4, 12.4 Hz, 1 H), 4.41-4.18 (m, |
| | | | | 3 H), 4.15-4.04 (m, 1 H), 3.71 (s, 3 H), 3.64-3.43 (m, |
| | | | | 3 H), 3.26 (br d, J = 11.2 Hz, 2 H), 3.22-2.99 (m, 6 H), |
| | | | | 2.96-2.86 (m, 3 H), 2.77-2.59 (m, 3 H), 2.31-2.13 (m, |
| | | | | 5 H), 2.04-1.90 (m, 5 H), 1.08-0.96 (m, 3 H) |
| I-928 | FR | FV | 803.0 | 12.43-11.85 (m, 1 H), 10.98-10.56 (m, 1 H), |
| | | | | 7.71-7.56 (m, 3 H), 7.51-7.32 (m, 3 H), 7.06-6.96 (m, 1 H), |
| | | | | 6.88 (d, J = 3.2 Hz, 2 H), 6.58-6.49 (m, 1 H), |
| | | | | 6.48-6.40 (m, 1 H), 6.30-6.06 (m, 2 H), 5.87-5.77 (m, 1 H), |
| | | | | 4.36 (s, 2 H), 4.19 (s, 2 H), 4.14-4.05 (m, 1 H), 3.62 |
| | | | | (d, J = 6.0 Hz, 4 H), 3.26 (d, J = 11.2 Hz, 3 H), 3.22-3.15 |
| | | | | (m, 2 H), 3.10-3.02 (m, 2 H), 2.79-2.65 (m, 4 H), |
| | | | | 2.61-2.54 (m, 1 H), 2.35-2.19 (m, 2 H), 2.15-2.06 (m, 1 H), |
| | | | | 1.94-1.79 (m, 5 H), 1.06-0.95 (m, 3 H) |
| I-939 | GM | AR | 872.4 | 12.19 (d, J = 8.8 Hz, 1 H), 10.85 (s, 1 H), 7.74-7.69 |
| | | | | (m, 1 H), 7.44-7.36 (m, 1 H), 7.27 (d, J = 8.4 Hz, 1 H), |
| | | | | 7.20-7.14 (m, 1 H), 6.99-6.90 (m, 1 H), 6.71 (d, J = 8.4 Hz, |
| | | | | 1 H), 6.59 (d, J = 14.4 Hz, 1 H), 6.50 (s, 1 H), 6.19 |
| | | | | (td, J = 2.0, 13.1 Hz, 1 H), 6.13 (s, 1 H), 5.14 (d, J = 6.8 Hz, |
| | | | | 1 H), 4.36 (q, J = 7.2 Hz, 4 H), 4.32-4.24 (m, 2 H), 3.82 |
| | | | | (s, 3 H), 3.66-3.55 (m, 2 H), 3.26 (s, 6 H), 3.12 (s, 6 H), |
| | | | | 2.97 (d, J = 7.2 Hz, 2 H), 2.86-2.76 (m, 1 H), 2.57 |
| | | | | (d, J = 2.8 Hz, 1 H), 2.32 (d, J = 1.6 Hz, 2 H), 2.26 |
| | | | | (s, 1 H), 2.18-2.11 (m, 1 H), 2.00-1.87 (m, 1 H) |
| I-940 | GO | AR | 808.3 | 12.27-12.13 (m, 1 H), 10.78 (s, 1 H), 7.76-7.67 (m, |
| | | | | 1 H), 7.49-7.36 (m, 3 H), 7.19 (t, J = 8.8 Hz, 1 H), |
| | | | | 7.07-6.99 (m, 1 H), 6.96-6.87 (m, 2 H), 6.55 (dd, J = 2.0, |
| | | | | 15.2 Hz, 1 H), 6.49-6.43 (m, 1 H), 6.21-6.14 (m, 2 H), |
| | | | | 5.85 (d, J = 7.6 Hz, 1 H), 4.40-4.35 (m, 3 H), |
| | | | | 4.34-4.25 (m, 2 H), 3.67-3.56 (m, 2 H), 3.32-3.31 (m, 6 H), |
| | | | | 3.22 (s, 4 H), 3.05 (s, 4 H), 2.98 (d, J = 7.6 Hz, 2 H), |
| | | | | 2.79-2.69 (m, 1 H), 2.61-2.54 (m, 1 H), 2.36-2.26 (m, 2 H), |
| | | | | 2.10 (td, J = 4.0, 8.8 Hz, 1 H), 1.86 (dq, J = 4.8, 12.4 |
| | | | | Hz, 1 H) |
| I-941 | GQ | AR | 826.3 | 12.24-12.14 (m, 1 H), 10.79 (br s, 1 H), 8.47-8.44 (m, |
| | | | | 1 H), 7.74-7.70 (m, 1 H), 7.44-7.35 (m, 2 H), |
| | | | | 7.09-7.04 (m, 1 H), 7.04-6.97 (m, 1 H), 6.92 (t, J = 9.2 Hz, |
| | | | | 1 H), 6.65 (br s, 1 H), 6.56 (dd, J = 2.4, 15.2 Hz, 1 H), |
| | | | | 6.48-6.44 (m, 1 H), 6.22-6.17 (m, 1 H), 6.14 (br s, 1 H), |
| | | | | 5.86 (br d, J = 7.6 Hz, 1 H), 4.37 (br d,.J = 6.8 Hz, 2 H), |
| | | | | 4.32-4.25 (m, 2 H), 3.67-3.57 (m, 2 H), 3.25 (br s, 6 H), |
| | | | | 3.17 (br d, J = 2.4 Hz, 3 H), 3.05 (br s, 6 H), 3.00-2.96 |
| | | | | (m, 2 H), 2.74 (dt, J = 6.0, 11.6 Hz, 1 H), 2.61-2.55 (m, |
| | | | | 1 H), 2.33 (br d, J = 1.6 Hz, 2 H), 2.13-2.07 (m, 1 H), |
| | | | | 1.92-1.81 (m, 1 H) |
| I-798 | EX | AK | 857.2 | 12.24-12.16 (m, 1 H), 10.85 (br s, 1 H), 8.46 (s, 1 H), |
| | | | | 8.13-8.09 (m, 1 H), 7.71-7.66 (m, 1 H), 7.43-7.35 (m, |
| | | | | 1 H), 7.09-7.05 (m, 1 H), 7.04-6.97 (m, 1 H), 6.70 |
| | | | | (d, J = 8.4 Hz, 1 H), 6.65 (br d, J = 1.6 Hz, 1 H), 6.59 |
| | | | | (d, J = 14.4 Hz, 1 H), 6.19-6.13 (m, 1 H), 5.14 |
| | | | | (br d, J = 6.4 Hz, 1 H), 4.65-4.60 (m, 2 H), 4.37 (br s, 1 H), |
| | | | | 4.33-4.25 (m, 2 H), 3.82 (s, 3 H), 3.67-3.58 (m, 2 H), 3.26 (br |
| | | | | s, 6 H), 3.20-3.15 (m, 2 H), 3.10 (br s, 6 H), 3.07 (br s, |
| | | | | 2 H), 2.85-2.77 (m, 1 H), 2.56 (br d, J = 2.8 Hz, 1 H), |
| | | | | 2.36-2.26 (m, 2 H), 2.17-2.11 (m, 1 H), 1.98-1.88 |
| | | | | (m, 1 H) |
| I-984 | GU | AR | 860.4 | 12.12-11.97 (m, 1 H), 10.89-10.77 (m, 1 H), |
| | | | | 7.75-7.65 (m, 1 H), 7.47-7.32 (m, 1 H), 7.14 (d, J = 8.4 Hz, |
| | | | | 1 H), 6.94-6.82 (m, 2 H), 6.67 (d, J = 8.0 Hz, 1 H), 6.59 |
| | | | | (d, J = 14.0 Hz, 1 H), 6.48 (s, 2 H), 6.23-6.07 (m, 2 H), |
| | | | | 5.13 (d, J = 6.8 Hz, 1 H), 4.36 (q, J = 7.2 Hz, 3 H), |
| | | | | 4.33-4.22 (m, 2 H), 3.82 (s, 3 H), 3.57 (t, J = 5.6 Hz, 2 H), |
| | | | | 3.37-3.33 (m, 4 H), 3.16 (s, 2 H), 3.07 (s, 6 H), 2.97 (q, |
| | | | | J = 6.8 Hz, 3 H), 2.81 (ddd, J = 5.2, 13.2, 17.6 Hz, 1 H), |
| | | | | 2.57 (s, 1 H), 2.48-2.45 (m, 1 H), 2.35-2.24 (m, 2 H), |
| | | | | 2.19-2.11 (m, 1 H), 1.99-1.87 (m, 1 H), 1.76-1.66 (m, |
| | | | | 1 H), 0.77-0.69 (m, 4 H) |
| I-981 | HI | AR | 859.3 | 12.06 (d, J = 8.0 Hz, 1 H), 10.86 (s, 1 H), 7.73-7.69 (m, |
| | | | | 1 H), 7.45-7.33 (m, 1 H), 7.12 (d, J = 8.4 Hz, 1 H), |
| | | | | 6.92-6.83 (m, 2 H), 6.75 (d, J = 6.8 Hz, 1 H), 6.50 (t, J = |
| | | | | 12.4 Hz, 3 H), 6.22-6.16 (m, 1 H), 6.15-6.11 (m, 1 H), |
| | | | | 5.29 (d, J = 6.4 Hz, 1 H), 4.36 (q, J = 7.6 Hz, 4 H), 4.29 |
| | | | | (s, 1 H), 3.84 (s, 1 H), 3.81 (s, 3 H), 3.64 (t, J = 5.6 Hz, |
| | | | | 1 H), 3.57 (t, J = 5.6 Hz, 1 H), 3.20-3.02 (m, 6 H), 2.97 |
| | | | | (q, J = 6.8 Hz, 3 H), 2.86-2.79 (m, 2 H), 2.78-2.72 (m, |
| | | | | 2 H), 2.57 (s, 1 H), 2.34-2.25 (m, 2 H), 2.17-2.11 (m, |
| | | | | 1 H), 2.01-1.92 (m, 1 H), 1.87 (s, 1 H), 1.81 (s, 3 H), |
| | | | | 1.74-1.68 (m, 1 H), 0.75-0.69 (m, 4 H) |
| I-977 | GX | GE | 816.5 | 12.20-12.07 (m, 1 H), 10.79 (s, 1 H), 8.20-8.08 (m, 1 |
| | | | | H), 7.75-7.65 (m, 1 H), 7.60-7.50 (m, 2 H), 7.46- |
| | | | | 7.34 (m, 2 H), 7.13-6.96 (m, 2 H), 6.9-6.82 (m, 1 |
| | | | | H), 6.56-6.48 (m, 1 H), 6.45-6.33 (m, 1 H), 6.15 (s, |
| | | | | 1 H), 6.88-6.80 (m, 1 H), 4.70-4.53 (m, 2 H), |
| | | | | 4.45-4.33 (m, 2 H), 4.30-4.18 (m, 1 H), 3.70-3.55 |
| | | | | (m, 2 H), 3.33-3.21 (m, 3 H), 3.17-3.06 (m, 3 H), 3.04 (s, |
| | | | | 3 H), 2.82-2.3 (m, 4 H), 2.57-2.51 (m, 1 H), 2.36-2.28 |
| | | | | (m, 2 H), 2.14-2.07 (m, 1 H), 1.90-1.86 (m, 3 H), |
| | | | | 1.82-1.79 (m, 1 H), 1.77-1.71 (m, 2 H), 1.62-1.4 |
| | | | | (m, 2 H) |
| I-968 | HJ | AH | 824.5 | 12.36-11.90 (m, I H), 10.89 (s, I H), 7.76-7.67 (m, |
| | | | | 1 H), 7.37 (s, 2 H), 7.33 (s, 1 H), 7.26 (s, 2 H), 7.13-7.02 |
| | | | | (m, 2 H), 6.92-6.79 (m, 1 H), 6.39 (br s, 1 H), 6.26-6.10 |
| | | | | (m, 2 H), 4.40-4.35 (m, 4 H), 4.33 (s, 3 H), 3.69-3.52 |
| | | | | (m, 2 H), 3.42-3.33 (m, 3 H), 3.24-3.01 (m, 6 H), |
| | | | | 3.01-2.96 (m, 2 H), 2.94-2.84 (m, 2 H), 2.82-2.73 |
| | | | | (m, 1 H), 2.69-2.60 (m, 2 H), 2.44-2.21 (m, 4 H), 2.18 |
| | | | | (s, 3 H), 2.02 (br s, 4 H) |
| I-758 | ID | AR | 845.3 | 12.27 (s, 1 H), 10.85 (s, 1 H), 7.75-7.67 (m, 1 H), 7.56 |
| | | | | (d, J = 8.8 Hz, 1 H), 7.51 (d, J = 2.0 Hz, 1 H), 7.43 (s, |
| | | | | 1 H), 7.39 (d, J = 15.2 Hz, 1 H), 7.11-6.99 (m, 1 H), |
| | | | | 6.67 (d, J = 8.0 Hz, I H), 6.65 (d, J = 2.8 Hz, I H), 6.59 |
| | | | | (d, J = 14.4 Hz, 1 H), 6.23-6.11 (m, 2 H), 5.14 (d, J = |
| | | | | 6.8 Hz, 1 H), 4.41-4.24 (m, 5 H), 3.82 (s, 3 H), |
| | | | | 3.67-3.55 (m, 2 H), 3.44 ( s, 4 H), 3.23-2.93 (m, 12 H), 2.81 |
| | | | | (ddd, J = 5.2, 13.2, 18.0, 1 H), 2.57 (d, J = 3.2 Hz, 1 H), |
| | | | | 2.33 (s, 1 H), 2.27 ( s, 1 H), 2.19-2.08 (m, 1 H), 1.93 |
| | | | | (dq, J = 4.4, 12.8 Hz, 1 H) |
| I-759 | IE | AR | 774.3 | 12.21 (br d, J = 1.6 Hz, 1 H), 10.84 (s, 1 H), 7.77-7.68 |
| | | | | (m, 1 H), 7.62 (d, J = 8.0 Hz, 2 H), 7.49-7.33 (m, 3 H), |
| | | | | 7.15-6.98 (m, 2 H), 6.88 (d, J = 2.8 Hz, 1 H), |
| | | | | 6.84-6.75 (m, 2 H), 6.24-6.12 (m, 2 H), 4.45-4.27 (m, 4 H), |
| | | | | 3.96-3.81 (m, 3 H), 3.69-3.52 (m, 2 H), 3.25-2.93 |
| | | | | (m, 8 H), 2.87-2.69 (m, 4 H), 2.57-2.52 (m, 1 H), |
| | | | | 2.40-2.24 (m, 2 H), 2.16 (dq, J = 4.0, 12.8 Hz, 1 H), |
| | | | | 2.04-1.87 (m, 3 H), 1.85-1.72 (m, 2 H) |
| I-760 | EX | IF | 786.9 | 13.81 (s, 1 H), 10.87 (s, 1 H), 8.11 (d, J = 12.0 Hz, 1 H), |
| | | | | 8.02 (d, J = 1.6 Hz, 1 H), 7.72-7.66 (m, 1 H), |
| | | | | 7.51-7.44 (m, 1 H), 7.14-7.06 (m, 2 H), 6.70 (d, J = 8.0 Hz, |
| | | | | 1 H), 6.59 (d, J = 14.4 Hz, 1 H), 6.23 ( d, J = 1.2 Hz, 1 H), |
| | | | | 5.15 (d, J = 6.8 Hz, 1 H), 4.62 (q, J = 7.2 Hz, 2 H), 4.36 |
| | | | | (d, J = 18.4 Hz, 2 H), 4.32-4.25 (m, 1 H), 3.82 (s, 3 H), |
| | | | | 3.63 (td, J = 5.6, 20.0 Hz, 2 H), 3.27 (s, 3 H), 3.11 |
| | | | | (d, J = 3.2 Hz, 6 H), 2.85-2.77 (m, 1 H), 2.56 (d, J = 2.8 Hz, |
| | | | | 1 H), 2.39-2.26 (m, 3 H), 2.16-2.10 (m, 1 H), 1.98- |
| | | | | 1.90 (m, 1 H) |
| I-761 | II | AK | 838.4 | 12.23 (d, J = 7.2 Hz, I H), 10.88 (s, 1 H), 8.19-8.03 (m, |
| | | | | 1 H), 7.72-7.63 (m, 3 H), 7.51 (d, J = 8.4 Hz, 2 H), |
| | | | | 7.13-7.01 (m, 1 H), 6.89 (s, 1 H), 6.71 (d, J = 8.0 Hz, 1 H), |
| | | | | 6.64-6.55 (m, 1 H), 6.18 (d, J = 7.2 Hz, 1 H), 5.18 (d, |
| | | | | J = 6.4 Hz, 1 H), 5.02-4.82 (m, 1 H), 4.67-4.59 (m, |
| | | | | 2 H), 4.45-4.34 (m, 2 H), 4.30 (dd, J = 6.0, 12.4 Hz, |
| | | | | 1 H), 3.86-3.80 (m, 3 H), 3.68-3.58 (m, 3 H), 3.40- |
| | | | | 3.38 (m, 6 H), 3.21 (s, 1 H), 3.11 (d, J = 6.4 Hz, 2 H), |
| | | | | 2.91-2.76 (m, 4 H), 2.55 (s, 2 H), 2.37-2.27 (m, 2 H), |
| | | | | 2.17-2.09 (m, 1 H), 1.97-1.92 (m, 2 H) |
| I-681 | KH | AR | 869.3 | 12.31-11.88 (m, 1 H), 11.12-10.64 (m, 1 H), 7.76 |
| | | | | 7.65 (m, 1 H), 7.45-7.36 (m, 1 H), 7.35-7.26 (m, |
| | | | | 3 H), 6.95-6.80 (m, 1 H), 6.71 (d, J = 8.0 Hz, 1 H), |
| | | | | 6.60 (d, J = 14.4 Hz, 1 H), 6.42-6.37 (m, 1 H), |
| | | | | 6.23-6.11 (m, 2 H), 5.19 (d, J = 7.2 Hz, 1 H), 4.39-4.28 |
| | | | | (m, 5 H), 3.84 (s, 3 H), 3.68-3.55 (m, 2 H), 3.54-3.46 (m, |
| | | | | 1 H), 3.24 (s, 2 H), 3.17-3.02 (m, 6 H), 3.00-2.96 (m, |
| | | | | 3 H), 2.92 (d, J = 14.4 Hz, 1 H), 2.86-2.78 (m, 1 H), |
| | | | | 2.58 (d, J = 2.8 Hz, 1 H), 2.35-2.25 (m, 3 H), 2.18 (s, |
| | | | | 3 H), 2.14 (d, J = 5.2 Hz, 1 H), 2.03-1.89 (m, 2 H) |
| I-682 | IU | AR | 836.4 | 12.32-12.04 (m, 1 H), 11.00-10.71 (m, 1 H), 7.77 (s, |
| | | | | 1 H), 7.59-7.51 (m, 1 H), 7.45-7.35 (m, 1 H), |
| | | | | 7.34-7.29 (m, 2 H), 7.07-6.98 (m, 1 H), 6.71-6.66 (m, |
| | | | | 1 H), 6.65-6.60 (m, 1 H), 6.49 (d, J = 12.8 Hz, 1 H), |
| | | | | 6.23-6.12 (m, 2 H), 5.32-5.20 (m, 1 H), 4.44-4.22 |
| | | | | (m, 6 H), 3.77 (d, J = 2.8 Hz, 3 H), 3.68-3.62 (m, 1 H), |
| | | | | 3.59 (t, J = 5.6 Hz, 1 H), 3.22-3.09 (m, 4 H), |
| | | | | 3.08-3.02 (m, 3 H), 3.01-2.93 (m, 4 H), 2.84-2.77 (m, |
| | | | | 1 H), 2.58-2.54 (m, 1 H), 2.34 (d, J = 1.6 Hz, 1 H), |
| | | | | 2.16-2.10 (m, 3 H), 1.97-1.89 (m, 3 H), 1.78 |
| | | | | (d, J = 8.4 Hz, 3 H) |
| I-686^{c,d} | JD | BK | 853.3 | 12.21 (s, 1 H), 11.09 (s, 1 H), 7.37 (dd, J = 7.2, 12.8 |
| | | | | Hz, 1 H), 7.09-6.92 (m, 5 H), 6.65 (d, J = 1.6 Hz, |
| | | | | 1 H), 6.19-6.11 (m, 1 H), 5.37 (dd, J = 5.6, 12.8 Hz, |
| | | | | 1 H), 4.45-4.31 (m, 2 H), 3.72 (d, J = 1.6 Hz, 1 H), |
| | | | | 3.69-3.64 (m, 2 H), 3.61 (s, 3 H), 3.37-3.32 (m, 6 H), |
| | | | | 3.25 (d, J = 8.8 Hz, 2 H), 3.11 (d, J = 7.2 Hz, 4 H), |
| | | | | 2.99-2.92 (m, 2 H), 2.91-2.84 (m, 1 H), 2.80 (d, J = 8.0 |
| | | | | Hz, I H), 2.68-2.59 (m, 4 H), 2.39-2.23 (m, 2 H), |
| | | | | 2.05-1.95 (m, 1 H), 1.05 (dd, J = 4.8, 6.0 Hz, 6 H), |
| | | | | 1.01 (d, J = 6.8 Hz, 3 H) |
| I-687 | JE | JF | 791.3 | 12.27-12.12 (m, 1 H), 10.96-10.84 (m, 1 H), 7.72 |
| | | | | (dd, J = 2.0, 11.2 Hz, 1 H), 7.62 (d, J = 8.0 Hz, 2 H), |
| | | | | 7.45-7.37 (m, 3 H), 7.08-6.99 (m, 1 H), 6.89-6.85 |
| | | | | (m, 1 H), 6.70 (d, J = 12.8 Hz, 2 H), 6.23-6.13 (m, |
| | | | | 2 H), 4.40-4.36 (m, 3 H), 4.31 (br s, 1 H), 4.09-4.02 |
| | | | | (m, 1 H), 3.94 (br d, J = 12.4 Hz, 2 H), 3.66-3.56 (m, |
| | | | | 2 H), 3.26-3.04 (m, 6 H), 3.00-2.76 (m, 7 H), 2.28 |
| | | | | (br s, 2 H), 2.17-2.06 (m, 1 H), 2.03-1.96 (m, 1 H), |
| | | | | 1.92 (br d, J = 11.2 Hz, 2 H), 1.82-1.71 (m, 2 H) |
| I-689 | JI | JK | 817.4 | 12.13 (d, J = 6.4 Hz, 1 H), 10.81 (s, 1 H), 8.14-8.06 |
| | | | | (m, 1 H), 7.71-7.64 (m, 1 H), 7.22-7.15 (m, 2 H), |
| | | | | 7.14-7.09 (m, 1 H), 7.04-6.91 (m, 3 H), 6.90-6.76 |
| | | | | (m, 1 H), 6.36 (s, 1 H), 6.19-6.08 (m, 1 H), 4.62 |
| | | | | (q, J = 7.2 Hz, 2 H), 4.41-4.27 (m, 2 H), 3.79 (dd, J = 4.8, |
| | | | | 11.8 Hz, 1 H), 3.68-3.52 (m, 2 H), 3.37 (s, 2 H), |
| | | | | 3.21-2.91 (m, 8 H), 2.71-2.57 (m, 5 H), 2.47 (s, 1 H), 2.33 |
| | | | | (s, 1 H), 2.30-2.23 (m, 1 H), 2.19 (dd, J = 4.0, 12.4 |
| | | | | Hz, 1 H), 2.12 (s, 3 H), 2.00 (td, J = 4.4, 8.8 Hz, 1 H), |
| | | | | 1.85 (d, J = 10.4 Hz, 2 H), 1.62 (d, J = 7.2 Hz, 2 H), |
| | | | | 1.49-1.30 (m, 3 H) |
| I-693 | JP | AR | 896.3 | 12.26-12.13 (m, 1 H), 10.87-10.80 (m, 1 H), |
| | | | | 7.75-7.66 (m, 1 H), 7.45-7.30 (m, 2 H), 7.03-6.95 (m, |
| | | | | 2 H), 6.65-6.59 (m, 1 H), 6.56-6.49 (m, 1 H), |
| | | | | 6.23-6.12 (m, 3 H), 4.91-4.84 (m, 1 H), 4.38-4.34 (m, |
| | | | | 3 H), 4.33-4.24 (m, 1 H), 4.21-4.14 (m, 1 H), |
| | | | | 3.80-3.76 (m, 3 H), 3.65-3.62 (m, 4 H), 3.62-3.54 |
| | | | | (m, 2 H), 3.22-3.16 (m, 2 H), 3.12-3.05 (m, 6 H), |
| | | | | 3.04-2.93 (m, 4 H), 2.78 (br d, J = 12.4 Hz, 1 H), |
| | | | | 2.58-2.55 (m, 1 H), 2.36-2.23 (m, 3 H), 2.13 (br s, I H), |
| | | | | 1.95-1.90 (m, 4 H) |
| I-694 | JS | AR | 910.3 | 12.25-12.03 (m, 1 H), 10.92-10.77 (m, 1 H), |
| | | | | 7.75-7.70 (m, 1 H), 7.45-7.37 (m, 1 H), 7.27 (ddd, J = 3.6, |
| | | | | 7.2, 14.0 Hz, 1 H), 6.99-6.91 (m, 1 H), 6.69-6.62 |
| | | | | (m, 3 H), 6.55 (d, J = 14.0 Hz, 1 H), 6.23-6.17 (m, |
| | | | | 1 H), 6.13 (br s, 1 H), 5.10 (d, J = 6.4 Hz, 1 H), 4.37 (br |
| | | | | d, J = 6.8 Hz, 3 H), 4.31-4.23 (m, 2 H), 3.80 (s, 3 H), |
| | | | | 3.65 (br t, J = 5.6 Hz, 1 H), 3.60-3.52 (m, 3 H), |
| | | | | 3.46-3.39 (m, 1 H), 3.23-3.04 (m, 6 H), 3.01-2.94 (m, |
| | | | | 5 H), 2.90 (br d, J = 5.2 Hz, 2 H), 2.83-2.76 (m, 1 H), |
| | | | | 2.56 (br d, J = 3.2 Hz, 1 H), 2.35-2.26 (m, 2 H), |
| | | | | 2.16-2.11 (m, 1 H), 1.99-1.92 (m, 1 H), 1.89 |
| | | | | (br t, J = 6.4 Hz, 2 H), 1.73 (br s, 4 H) |
| I-695 | JV | AR | 924.4 | 12.27-12.05 (m, 1 H), 10.95-10.72 (m, 1 H), |
| | | | | 7.75-7.67 (m, 1 H), 7.47-7.23 (m, 2 H), 7.08-6.90 (m, |
| | | | | 2 H), 6.73-6.59 (m, 2 H), 6.58-6.50 (m, 1 H), |
| | | | | 6.22-6.11 (m, 2 H), 5.21-4.98 (m, 1 H), 4.39-4.33 (m, |
| | | | | 3 H), 4.31-4.21 (m, 2 H), 3.83-3.74 (m, 3 H), |
| | | | | 3.66-3.55 (m, 2 H), 3.20-3.15 (m, 2 H), 3.14-3.02 |
| | | | | (m, 8 H), 3.01-2.94 (m, 3 H), 2.91 (br s, 3 H), 2.85 |
| | | | | (br s, 2 H), 2.33-2.25 (m, 2 H), 2.16-2.09 (m, 1 H), |
| | | | | 1.96-1.86 (m, 1 H), 1.70-1.59 (m, 8 H) |
| I-696 | JY | AR | 939.5 | 12.28-12.11 (m, 1 H), 10.93-10.78 (m, 1 H), |
| | | | | 7.75-7.69 (m, 1 H), 7.45-7.32 (m, 2 H), 7.05-6.95 (m, |
| | | | | 2 H), 6.62 (s, 2 H), 6.58-6.51 (m, 1 H), 6.23-6.12 (m, |
| | | | | 2 H), 5.11-5.06 (m, 1 H), 4.36 (d, J = 6.4 Hz, 3 H), |
| | | | | 4.33-4.21 (m, 2 H), 3.79 (s, 3 H), 3.67-3.56 (m, 2 H), |
| | | | | 3.26-3.19 (m, 3 H), 3.17-3.10 (m, 6 H), 3.09-2.93 |
| | | | | (m, 5 H), 2.85-2.77 (m, 1 H), 2.71 (s, 4 H), 2.68-2.61 |
| | | | | (m, 2 H), 2.56 (s, 1 H), 2.39-2.25 (m, 3 H), 2.12 (s, |
| | | | | 1 H), 1.88 (d, J = 11.6 Hz, 3 H), 1.68-1.56 (m, 2 H) |
| I-678 | IL | AK | 819.6 | 12.62-11.71 (m, 1 H), 11.34-10.44 (m, 1 H), 8.15-8.08 |
| | | | | (m, 1 H), 7.68 (d, J = 13.2 Hz, 1 H), 7.63 (d, J = |
| | | | | 7.6 Hz, 2 H), 7.48 (d, J = 8.0 Hz, 2 H), 7.10-6.97 (m, |
| | | | | 1 H), 6.90-6.86 (m, 1 H), 6.68 (d, J = 7.2 Hz, 1 H), |
| | | | | 6.49 (d, J = 13.2 Hz, 1 H), 6.16 (d, J = 1.6 Hz, 1 H), |
| | | | | 5.27 (d, J = 6.8 Hz, 1 H), 4.66-4.59 (m, 2 H), |
| | | | | 4.40-4.33 (m, 2 H), 4.31-4.25 (m, 1 H), 3.76 (s, 3 H), |
| | | | | 3.67-3.58 (m, 2 H), 3.23-3.06 (m, 8 H), 3.02-2.97 (m, |
| | | | | 2 H), 2.84-2.75 (m, 1 H), 2.59-2.52 (m, 1 H), |
| | | | | 2.40-2.32 (m, 2 H), 2.27 (d, J = 4.0 Hz, 1 H), 2.12 |
| | | | | (d, J = 10.4 Hz, 3 H), 1.93-1.76 (m, 6 H) |
| I-679 | IM | AK | 819.0 | 12.25-12.09 (m, 1 H), 10.97-10.72 (m, 1 H), |
| | | | | 8.14-8.08 (m, 1 H), 7.68 (s, 1 H), 7.60 (br d, J = 8.0 Hz, 2 H), |
| | | | | 7.40 (d, J = 8.4 Hz, 2 H), 7.08-6.99 (m, 1 H), 6.87 (br |
| | | | | s, 1 H), 6.80-6.75 (m, 1 H), 6.53-6.46 (m, 1 H), |
| | | | | 6.20-6.14 (m, 1 H), 5.27 (d, J = 7.2 Hz, 1 H), 4.66-4.59 |
| | | | | (m, 2 H), 4.40-4.37 (m, 1 H), 4.36-4.26 (m, 2 H), |
| | | | | 3.82 (s, 3 H), 3.68-3.59 (m, 2 H), 3.20-3.06 (m, 7 H), |
| | | | | 2.86-2.77 (m, 2 H), 2.70-2.66 (m, 1 H), 2.59-2.54 |
| | | | | (m, 1 H), 2.42-2.24 (m, 3 H), 2.18-2.11 (m, 1 H), |
| | | | | 2.01-1.93 (m, 3 H), 1.90-1.84 (m, 2 H), 1.76-1.63 |
| | | | | (m, 4 H) |
| I-680 | IQ | AR | 869.4 | 13.13-11.41 (m, 1 H), 11.37-9.90 (m, 1 H), |
| | | | | 7.81-7.59 (m, 1 H), 7.45-7.32 (m, 2 H), 7.28 (s, 2 H), |
| | | | | 6.95-6.83 (m, 1 H), 6.72 (d, J = 8.0 Hz, 1 H), 6.60 |
| | | | | (d, J = 14.0 Hz, 1 H), 6.44-6.35 (m, 1 H), 6.24-6.10 (m, |
| | | | | 2 H), 5.16 (d, J = 6.8 Hz, 1 H), 4.41-4.27 (m, 5 H), |
| | | | | 3.88-3.79 (m, 3 H), 3.64 (t, J = 5.6 Hz, 1 H), 3.59- |
| | | | | 3.46 (m, 2 H), 3.24 (d, J = 12.0 Hz, 2 H), 3.22-3.02 |
| | | | | (m, 6 H), 3.02-2.96 (m, 3 H), 2.96-2.86 (m, 1 H), |
| | | | | 2.86-2.75 (m, 1 H), 2.60-2.53 (m, 1 H), 2.36-2.24 |
| | | | | (m, 3 H), 2.16 (s, 3 H), 2.16-2.08 (m, 1 H), 2.02-1.89 |
| | | | | (m, 2 H) |
| I-691 | FO | IF | 723.3 | 13.97-13.64 (m, 1 H), 10.91-10.71 (m, 1 H), |
| | | | | 8.14-8.06 (m, 1 H), 8.00 (s, 1 H), 7.68 (d, J = 12.0 Hz, 1 H), |
| | | | | 7.61-7.51 (m, 1 H), 7.38-7.29 (m, 2 H), 7.16-7.03 |
| | | | | (m, 3 H), 7.01-6.95 (m, 1 H), 6.22 (s, 1 H), 4.65-4.59 |
| | | | | (m, 2 H), 4.37 (d, J = 18.0 Hz, 2 H), 3.82 (dd, J = 4.8, |
| | | | | 11.6 Hz, 1 H), 3.68-3.59 (m, 2 H), 3.49 (d, J = 11.6 |
| | | | | Hz, 2 H), 3.12-3.07 (m, 2 H), 2.81 (t, J = 10.4 Hz, |
| | | | | 3 H), 2.70-2.62 (m, 1 H), 2.36 (d, J = 1.6 Hz, 1 H), |
| | | | | 2.29 (s, 1 H), 2.24-2.14 (m, 1 H), 2.11-1.76 (m, 6 H) |
| I-692 | JM | AR | 858.4 | 12.12 (d, J = 8.4 Hz, 1 H), 10.84 (s, 1 H), 7.96 |
| | | | | (t, J = 7.2 Hz, 1 H), 7.78-7.68 (m, 1 H), 7.64 (t, J = 8.4 Hz, |
| | | | | 1 H), 7.57-7.47 (m, 2 H), 7.47-7.33 (m, 1 H), 7.08 |
| | | | | (d, J = 8.8 Hz, 2 H), 7.02-6.91 (m, 1 H), 6.88 (s, 1 H), |
| | | | | 6.34-6.28 (m, 1 H), 6.25-6.20 (m, 1 H), 6.18-6.12 |
| | | | | (m, 1 H), 4.80-4.63 (m, 1 H), 4.43-4.35 (m, 3 H), |
| | | | | 4.30 (s, 1 H), 3.72 (s, 4 H), 3.67-3.56 (m, 2 H), 3.24 |
| | | | | (s, 4 H), 3.23-3.02 (m, 6 H), 3.02-2.95 (m, 2 H), |
| | | | | 2.85-2.71 (m, 1 H), 2.57-2.51 (m, 2 H), 2.38-2.23 |
| | | | | (m, 2 H), 2.19-1.98 (m, 2 H), 1.92 (s, 4 H) |
| I-697 | KB | AR | 921.7 | 12.17-12.05 (m, 1 H), 10.85 (s, 1 H), 7.74-7.68 |
| | | | | (m, 1 H), 7.44-7.33 (m, 2 H), 6.97-6.83 (m, 3 H), |
| | | | | 6.63-6.58 (m, 2 H), 6.55 (d, J = 14.8 Hz, 1 H), 6.22-6.16 |
| | | | | (m, 1 H), 6.15-6.08 (m, 1 H), 5.08 (d, J = 6.8 Hz, 1 H), |
| | | | | 4.31-4.21 (m, 2 H), 3.86 (d, J = 11.6 Hz, 2 H), 3.78 |
| | | | | (s, 3 H), 3.66-3.56 (m, 2 H), 3.21-2.87 (m, 14 H), |
| | | | | 2.85-2.74 (m, 4 H), 2.65 (s, 4 H), 2.55 (d, J = 2.8 Hz, |
| | | | | 1 H), 2.44 (t, J = 10.8 Hz, I H), 2.37-2.22 (m, 2 H), |
| | | | | 2.18-2.07 (m, 1 H), 1.98-1.82 (m, 3 H), 1.62-1.46 |
| | | | | (m, 2 H) |
| I-698 | BT | KD | 776.4 | 12.09 (s, 1 H), 10.92-10.75 (m, 1 H), 7.76-7.67 (m, |
| | | | | 1 H), 7.62 (d, J = 8.0 Hz, 2 H), 7.49-7.37 (m, 3 H), |
| | | | | 7.11-7.03 (m, 3 H), 6.99 (d, J = 8.0 Hz, 1 H), 6.86 (s, |
| | | | | 1 H), 6.20 (d, J = 10.4 Hz, 1 H), 4.50 (d, J = 10.8 Hz, |
| | | | | 1 H), 4.36 (q, J = 6.8 Hz, 2 H), 3.93-3.76 (m, 2 H), |
| | | | | 3.50 (d, J = 11.2 Hz, 2 H), 3.25 (d, J = 13.2 Hz, 1 H), |
| | | | | 3.20-3.01 (m, 6 H), 2.93 (td, J = 7.6, 14.4 Hz, 3 H), |
| | | | | 2.86-2.72 (m, 4 H), 2.69-2.54 (m, 2 H), 2.22 |
| | | | | (q, J = 4.0, 12.4 Hz, 1 H), 2.02 (td, J = 4.4, 8.4 Hz, 1 H), |
| | | | | 1.97-1.87 (m, 6 H), 1.77 (d, J = 12.0 Hz, 1 H), 1.61-1.35 |
| | | | | (m, 1 H) |
| I-701^{c,d} | GJ | KD | 905.4 | 12.12-11.98 (m, 1 H), 10.85 (s, 1 H), 7.71 (dd, J = |
| | | | | 2.0, 10.4 Hz, 1 H), 7.61-7.51 (m, 2 H), 7.46-7.35 |
| | | | | (m, 1 H), 7.19-7.08 (m, 2 H), 7.02-6.97 (m, 1 H), |
| | | | | 6.87-6.80 (m, 1 H), 6.64-6.52 (m, 2 H), 6.24-6.13 |
| | | | | (m, 1 H), 5.19-5.07 (m, 1 H), 4.54-4.43 (m, 1 H), |
| | | | | 4.39-4.32 (m, 2 H), 4.30-4.22 (m, 1 H), 3.95 |
| | | | | (br d, J = 8.8 Hz, 2 H), 3.87 (br s, 1 H), 3.82-3.78 (m, 3 H), |
| | | | | 3.71-3.60 (m, 2 H), 3.25 (br s, 3 H), 3.23-3.01 (m, |
| | | | | 10 H), 2.99-2.87 (m, 3 H), 2.86-2.60 (m, 5 H), 2.57 |
| | | | | (br s, 1 H), 2.30-2.14 (m, 2 H), 2.14-2.07 (m, 1 H), |
| | | | | 1.97-1.82 (m, 5 H), 1.80-1.74 (m, 1 H), 1.54-1.39 |
| | | | | (m, 1 H) |
| I-724^{c} | EX | KC | 858.2 | 12.10 (s, 1 H), 10.85 (s, 1 H), 7.71 (dd, J = 1.8, 10.0 |
| | | | | Hz, 1 H), 7.41 (d, J = 16.8 Hz, 1 H), 7.35 (dt, J = 3.6, |
| | | | | 6.4 Hz, 1 H), 7.09-7.02 (m, 2 H), 6.70 (d, J = 8.0 Hz, |
| | | | | 1 H), 6.62 (s, 1 H), 6.59 (d, J = 14.0 Hz, 1 H), 6.20 |
| | | | | (d, J = 10.4 Hz, 1 H), 5.14 (d, J = 6.8 Hz, 1 H), 4.49 |
| | | | | (d, J = 12.0 Hz, I H), 4.35 (q, J = 6.4 Hz, 2 H), 4.31-4.24 |
| | | | | (m, 1 H), 3.89 (d, J = 12.8 Hz, 1 H), 3.82 (s, 3 H), 3.26 |
| | | | | (s, 5 H), 3.10 (s, 6 H), 3.07-3.00 (m, 3 H), 2.98-2.87 |
| | | | | (m, 3 H), 2.86-2.73 (m, 2 H), 2.57 (d, J = 3.2 Hz, 1 H), |
| | | | | 2.54-2.52 (m, 1 H), 2.18-2.10 (m, 1 H), 1.99-1.85 |
| | | | | (m, 3 H), 1.76 (d, J = 11.6 Hz, 1 H), 1.56-1.37 (m, |
| | | | | 1 H) |
| I-688 | JI | JH | 833.4 | 12.10-11.94 (m, 1 H), 10.94-10.70 (m, 1 H), 8.16-8.03 |
| | | | | (m, 1 H), 7.78-7.60 (m, 1 H), 7.25 (br d, J = 7.6 |
| | | | | Hz, 1 H), 7.06-6.83 (m, 6 H), 6.56-6.39 (m, 1 H), |
| | | | | 6.23-6.06 (m, 1 H), 4.72-4.57 (m, 2 H), 4.42-4.26 |
| | | | | (m, 2 H), 3.86-3.77 (m, 1 H), 3.77-3.70 (m, 3 H), |
| | | | | 3.68-3.56 (m, 2 H), 3.40-3.37 (m, 1 H), 3.31-2.92 |
| | | | | (m, 9 H), 2.79-2.54 (m, 6 H), 2.35 (br s, 1 H), |
| | | | | 2.31-2.13 (m, 2 H), 2.03-1.95 (m, I H), 1.92-1.80 (m, |
| | | | | 2 H), 1.73-1.61 (m, 2 H), 1.51-1.35 (m, 3 H) |
| I-702^{c} | EX | KD | 858.5 | 12.19-12.06 (m, 1 H), 10.87 (s, 1 H), 7.71 |
| | | | | (dd, J = 2.0, 10.0 Hz, 1 H), 7.48-7.29 (m, 2 H), 7.13-7.00 |
| | | | | (m, 2 H), 6.70 (d,.J = 8.0 Hz, 1 H), 6.63 (s, 1 H), 6.59 |
| | | | | (d, J = 14.4 Hz, 1 H), 6.20 (td, J = 2.0, 10.4 Hz, 1 H), |
| | | | | 5.15 (d, J = 7.2 Hz, 1 H), 4.48 (dd, J = 1.6, 11.6 Hz, |
| | | | | 1 H), 4.38-4.25 (m, 3 H), 3.92-3.85 (m, 1 H), 3.82 (s, |
| | | | | 3 H), 3.26 (s, 5 H), 3.21-3.16 (m, 2 H), 3.10 ( s, 5 H), |
| | | | | 3.06-2.99 (m, 3 H), 2.98-2.88 (m, 3 H), 2.86-2.73 |
| | | | | (m, 2 H), 2.56 (d, J = 3.2 Hz, 1 H), 2.18-2.09 (m, 1 H), |
| | | | | 1.98-1.84 (m, 3 H), 1.80-1.72 (m, 1 H), 1.54-1.38 |
| | | | | (m, 1 H) |
| I-683 | IX | AR | 851.3 | 12.18 (br d, J = 8.4 Hz, 1 H), 10.89 (s, 1 H), |
| | | | | 7.76-7.70 (m, 1 H), 7.46-7.36 (m, 1 H), 7.11-7.06 (m, |
| | | | | 1 H), 7.05-6.98 (m, 1 H), 6.91-6.78 (m, 2 H), 6.53 |
| | | | | (d, J = 12.8 Hz, 1 H), 6.44 (br s, 1 H), 6.23-6.17 (m, |
| | | | | 1 H), 6.15 (br d, J = 10.4 Hz, 1 H), 5.31 (br d, J = 7.2 |
| | | | | Hz, 1 H), 4.40-4.35 (m, 3 H), 4.32 (br d, J = 16.8 Hz, |
| | | | | 2 H), 3.83 (s, 3 H), 3.64 (br t, J = 5.6 Hz, 1 H), 3.58 (br |
| | | | | t, J = 5.6 Hz, 1 H), 3.52 (br d, J = 10.8 Hz, 2 H), |
| | | | | 3.18-2.95 (m, 8 H), 2.89-2.77 (m, 4 H), 2.57 (br d, J = 3.2 |
| | | | | Hz, 1 H), 2.33 (br s, 1 H), 2.26 (br s, 1 H), 2.18-2.11 |
| | | | | (m, 1 H), 2.08-2.03 (m, 3 H), 2.02-1.90 (m, 3 H), |
| | | | | 1.86-1.78 (m, 2 H) |
| I-690^{c} | FC | IF | 822.8 | 13.97-13.62 (m, 1 H), 10.89 (s, 1 H), 8.10 (d, J = 10.4 |
| | | | | Hz, 1 H), 7.90 (s, 1 H), 7.68 (d, J = 11.2 Hz, 1 H), 7.57 |
| | | | | (d, J = 8.0 Hz, 1 H), 7.35 (dd, J = 7.2, 13.2 Hz, 1 H), |
| | | | | 7.21 (t, J = 7.6 Hz, 2 H), 7.09-6.95 (m, 2 H), 6.23 (d, |
| | | | | J = 8.0 Hz, 1 H), 4.62 (q, J = 7.2 Hz, 2 H), 4.40-4.32 |
| | | | | (m, 3 H), 4.25 (s, 3 H), 3.67-3.58 (m, 2 H), 3.30-3.25 |
| | | | | (m, 3 H), 3.17 (s, 4 H), 3.10 (q, J = 7.2 Hz, 2 H), 2.72 |
| | | | | (s, 4 H), 2.69-2.58 (m, 3 H), 2.40-2.26 (m, 3 H), |
| | | | | 2.23-2.12 (m, 1 H) |
| I-699^{c,d} | FU | KF | 792.3 | 12.19-11.99 (m, 1 H), 10.90-10.66 (m, 1 H), 8.11 |
| | | | | (d, J = 11.8 Hz, 1 H), 7.73-7.66 (m, 1 H), 7.61 |
| | | | | (d, J = 8.0 Hz, 2 H), 7.43 (d, J = 8.0 Hz, 2 H), 7.08 |
| | | | | (t, J = 5.2 Hz, 1 H), 6.91 (t, J = 9.6 Hz, 1 H), 6.86 (s, 1 H), |
| | | | | 6.57-6.50 (m, 1 H), 6.46 (d, J = 8.0 Hz, 1 H), 5.85-5.79 |
| | | | | (m, 1 H), 4.62 (q, J = 6.8 Hz, 2 H), 4.54-4.46 (m, 1 H), |
| | | | | 4.31-4.24 (m, 1 H), 3.96-3.88 (m, 1 H), 3.21 |
| | | | | (d, J = 1.6 Hz, 4 H), 3.15-2.99 (m, 8 H), 2.78-2.68 (m, 4 H), |
| | | | | 2.63-2.59 (m, 1 H), 2.14-2.07 (m, 1 H), 1.86 (s, 7 H), |
| | | | | 1.85-1.75 (m, 2 H), 1.59-1.43 (m, 1 H) |
| I-700^{c} | FK | KD | 858.3 | 12.13 (s, 1 H), 10.86 (s, 1 H), 7.71 (J = 2.0, 10.0 Hz, |
| | | | | 1 H), 7.44-7.38 (m, 1 H), 7.33-7.26 (m, 1 H), |
| | | | | 7.05-6.99 (m, 2 H), 6.71 (d, J = 8.0 Hz, 1 H), 6.64 (s, 1 H), |
| | | | | 6.59 (d, J = 14.4 Hz, 1 H), 6.20 (td, J = 2.0, 10.4 Hz, |
| | | | | 1 H), 5.14 (d, J = 6.4 Hz, 1 H), 4.49 (d, J = 10.8 Hz, |
| | | | | 1 H), 4.38-4.33 (m, 2 H), 4.31-4.23 (m, 1 H), 3.93- |
| | | | | 3.85 (m, 1 H), 3.82 (s, 3 H), 3.31-3.21 (m, 6 H), 3.12 |
| | | | | (s, 6 H), 3.05 (d, J = 11.4 Hz, 2 H), 2.98-2.87 (m, |
| | | | | 3 H), 2.86-2.71 (m, 2 H), 2.56 (d, J = 3.2 Hz, 1 H), |
| | | | | 2.54-2.51 (m, 1 H), 2.17-2.09 (m, 1 H), 1.98-1.85 |
| | | | | (m, 3 H), 1.80-1.72 (m, 1 H), 1.56-1.40 (m, 1 H) |
| I-725^{c,e} | KK | KD | 892.3 | 12.15 (s, 1 H), 11.07 (s, 1 H), 7.72 (dd, J = 2.0, 9.2 Hz, |
| | | | | 1 H), 7.56 (d, J = 6.8 Hz, 1 H), 7.43 (d, J = 4.0 Hz, |
| | | | | 1 H), 7.40 (s, 1 H), 7.37-7.27 (m, 2 H), 7.05-7.00 (m, |
| | | | | 1 H), 6.97 (d, J = 7.2 Hz, 1 H), 6.50 (t, J = 2.4 Hz, 1 H), |
| | | | | 6.24-6.16 (m, 1 H), 5.74 (dd, J = 5.2, 11.6 Hz, 1 H), |
| | | | | 4.49 (d, J = 12.4 Hz, 1 H), 4.40-4.32 (m, 2 H), 3.89 |
| | | | | (d, J = 12.3 Hz, 1 H), 3.26-3.01 (m, 12 H), 2.97-2.77 |
| | | | | (m, 7 H), 2.73 (d, J = 14.0 Hz, 2 H), 2.68 (s, 3 H), |
| | | | | 2.64-2.52 (m, 1 H), 2.45-2.38 (m, 1 H), 2.23 |
| | | | | (dd, J = 5.2, 10.0 Hz, 1 H), 1.96-1.72 (m, 7 H), |
| | | | | 1.57-1.38 (m, 1 H) |
| I-727 | KP | AR | 876.4 | 12.23-12.07 (m, 1 H), 10.82 (s, 1 H), 7.76-7.69 (m, |
| | | | | 1 H), 7.46-7.33 (m, 2 H), 7.07-6.84 (m, 6 H), 6.61 |
| | | | | (br d, J = 2.4 Hz, 1 H), 6.23-6.16 (m, 1 H), 6.15- |
| | | | | 6.09 (m, 1 H), 4.41-4.28 (m, 4 H), 3.80 (dd, J = 4.8, |
| | | | | 11.6 Hz, 1 H), 3.67-3.55 (m, 2 H), 3.42 (br d, J = 11.2 |
| | | | | Hz, 2 H), 3.25 (br s, 4 H), 3.20-3.03 (m, 5 H), 3.03-2.92 |
| | | | | (m, 3 H), 2.72-2.61 (m, 7 H), 2.47-2.14 (m, 5 H), |
| | | | | 2.05-1.95 (m, 1 H), 1.91 (br d, J = 11.2 Hz, 2 H), |
| | | | | 1.70-1.55 (m, 2 H) |
| I-728 | JE | KR | 812.0 | 12.42-12.10 (m, 1 H), 10.97-10.71 (m, 1 H), |
| | | | | 8.01-7.85 (m, 1 H), 7.78-7.65 (m, 1 H), 7.56-7.49 |
| | | | | (m, 1 H), 7.47-7.36 (m, 1 H), 7.35-7.27 (m, 1 H), |
| | | | | 7.08-6.97 (m, 2 H), 6.70-6.61 (m, 1 H), 6.24-6.11 (m, |
| | | | | 2 H), 4.37 (br d, J = 6.8 Hz, 3 H), 4.33-4.21 (m, 1 H), |
| | | | | 3.95-3.85 (m, 1 H), 3.71-3.63 (m, 1 H), 3.62-3.44 |
| | | | | (m, 6 H), 3.26-2.89 (m, 10 H), 2.75-2.67 (m, 1 H), |
| | | | | 2.57 (br d, J = 3.2 Hz, 1 H), 2.41-2.20 (m, 4 H), |
| | | | | 2.07-1.94 (m, 1 H) |
| I-729^{c,d} | KS | AK | 811.3 | 12.34-12.19 (m, 1 H), 10.89-10.82 (m, 1 H), |
| | | | | 8.51-8.42 (m, 1 H), 8.16-8.08 (m, 1 H), 7.69 |
| | | | | (br d, J = 11.6 Hz, 1 H), 7.41-7.37 (m, 1 H), 7.31 (br s, 1 H), |
| | | | | 7.11-7.03 (m, 3 H), 7.02-6.98 (m, 1 H), 6.71-6.62 (m, |
| | | | | 1 H), 6.23-6.11 (m, 1 H), 4.63 (q, J = 7.6 Hz, 2 H), |
| | | | | 4.40-4.30 (m, 2 H), 3.93-3.86 (m, 1 H), 3.67-3.57 |
| | | | | (m, 4 H), 3.22-3.14 (m, 3 H), 3.13-3.07 (m, 4 H), |
| | | | | 3.07-2.98 (m, 3 H), 2.98-2.92 (m, 2 H), 2.70-2.64 |
| | | | | (m, 1 H), 2.35 (br d, J = 4.4 Hz, 1 H), 2.31-2.22 |
| | | | | (m, 2 H), 2.07-2.01 (m, 1 H), 1.96-1.84 (m, 4 H) |
| I-730 | KV | AR | 894.4 | 1.60-1.68 (m, 2 H) 1.85-1.95 (m, 2 H) 1.88-1.93 |
| | | | | (m, 1 H) 1.95-2.05 (m, 1 H) 2.12-2.36 (m, 4 H) |
| | | | | 2.60-2.74 (m, 7 H) 2.94-3.01 (m, 3 H) 3.07-3.17 |
| | | | | (m, 9 H) 3.38-3.46 (m, 2 H) 3.55-3.67 (m, 2 H) |
| | | | | 3.74-3.84 (m, 1 H) 4.22-4.45 (m, 4 H) 6.08-6.30 |
| | | | | (m, 2 H) 6.63 (br s, 1 H) 6.88-7.04 (m, 5 H) 7.28 |
| | | | | 7.44 (m, 2 H) 7.69-7.79 (m, 1 H) 10.72-10.85 (m, |
| | | | | 1 H) 12.14-12.30 (m, 1 H) |
| I-732 | KZ | KD | 857.4 | 12.10 (s, 1 H), 10.86 (s, 1 H), 7.71 (dd, J = 2.0, 10.0 |
| | | | | Hz, 1 H), 7.41 (dd, J = 1.2, 16.0 Hz, 1 H), 7.34 |
| | | | | (ddd, J = 2.8, 7.2, 12.8 Hz, 1 H), 7.08-7.00 (m, 2 H), 6.79 |
| | | | | (d, J = 7.2 Hz, 1 H), 6.62 (s, 1 H), 6.52 (d. J = 12.8 Hz, |
| | | | | 1 H), 6.20 (td, J = 2.0, 10.4 Hz, 1 H), 5.30 (d, J = 6.8 |
| | | | | Hz, 1 H), 4.48 (d, J = 12.4 Hz, 1 H), 4.39-4.27 (m, |
| | | | | 3 H), 3.92-3.84 (m, 1 H), 3.82 (s, 3 H), 3.58 (d, J = |
| | | | | 10.8 Hz, 2 H), 3.12-3.12 (m, 1 H), 3.21-3.01 (m, |
| | | | | 6 H), 2.97-2.72 (m, 8 H), 2.60-2.55 (m, 1 H), |
| | | | | 2.18-2.09 (m, 1 H), 1.79 (s, 8 H), 1.58-1.38 (m, 1 H) |
| I-733^{c,d} | LA | AR | 894.5 | 12.34-12.12 (m, 1 H), 10.82 (s, 1 H), 7.81-7.61 (m, |
| | | | | 1 H), 7.47-7.25 (m, 2 H), 7.07-6.91 (m, 5 H), 6.66 |
| | | | | (br d, J = 2.4 Hz, 1 H), 6.28-6.05 (m, 2 H), |
| | | | | 4.41-4.29 (m, 4 H), 3.86-3.76 (m, 1 H), 3.68-3.54 (m, |
| | | | | 2 H), 3.42 (br d, J = 11.2 Hz, 2 H), 3.23-3.03 (m, |
| | | | | 10 H), 3.02-2.93 (m, 3 H), 2.77-2.63 (m, 7 H), |
| | | | | 2.36-2.15 (m, 4 H), 2.05-1.87 (m, 3 H), 1.69-1.56 |
| | | | | (m, 2 H) |
| I-734 | LC | AR | 894.5 | 12.23-12.16 (m, 1 H), 10.81 (s, 1 H), 7.75-7.68 (m, |
| | | | | 1 H), 7.46-7.30 (m, 2 H), 7.05-6.96 (m, 5 H), 6.63 (s, |
| | | | | 1 H), 6.21-6.10 (m, 2 H), 4.39-4.28 (m, 4 H), 3.80 |
| | | | | (J = 4.8, 12.1 Hz, 1 H), 3.65-3.52 (m, 4 H), 3.23-3.04 |
| | | | | (m, 6 H), 3.02 (s, 6 H), 2.81-2.63 (m, 8 H), 2.42-2.30 |
| | | | | (m, 2 H), 2.28-2.13 (m, 2 H), 2.05-1.91 (m, 3 H), |
| | | | | 1.69-1.58 (m, 2 H) |
| I-774 | IL | AR | 818.3 | 12.27-12.08 (m, 1 H), 10.95-10.73 (m, 1 H), |
| | | | | 7.74-7.69 (m, 1 H), 7.63 (br d, J = 8.0 Hz, 2 H), 7.48 |
| | | | | (d, J = 8.4 Hz, 2 H), 7.44-7.36 (m, 1 H), 7.09-6.99 (m, 1 H), |
| | | | | 6.88 (d, J = 2.4 Hz, I H), 6.68 (br d, J = 7.2 Hz, I H), |
| | | | | 6.49 (d, J = 13.2 Hz, 1 H), 6.22-6.14 (m, 2 H), 5.29- |
| | | | | 5.24 (m, 1 H), 4.37 (br t, J = 6.8 Hz, 3 H), 4.32-4.26 |
| | | | | (m, 2 H), 3.77-3.74 (m, 3 H), 3.66-3.57 (m, 2 H), |
| | | | | 3.23-3.09 (m, 4 H), 3.06-2.94 (m, 6 H), 2.84-2.76 |
| | | | | (m, 1 H), 2.58-2.53 (m, 1 H), 2.35-2.26 (m, 2 H), |
| | | | | 2.15-2.08 (m, 3 H), 1.95-1.80 (m, 5 H), 1.78-1.72 |
| | | | | (m, 2 H) |
| I-707 | IM | AR | 818.4 | 12.25-12.10 (m, 1 H), 10.94-10.77 (m, 1 H), |
| | | | | 7.77-7.67 (m, 1 H), 7.60 (d, J = 8.0 Hz, 2 H), 7.44-7.37 |
| | | | | (m, 3 H), 7.07-6.99 (m, 1 H), 6.88 (d, J = 2.8 Hz, 1 H), |
| | | | | 6.79-6.75 (m, 1 H), 6.50 (d, J = 12.8 Hz, 1 H), |
| | | | | 6.22-6.13 (m, 2 H), 5.27 (d, J = 6.8 Hz, 1 H), 4.40-4.29 |
| | | | | (m, 5 H), 3.82 (s, 3 H), 3.67-3.56 (m, 2 H), 3.26-3.02 (m, |
| | | | | 6 H), 3.01-2.96 (m, 2 H), 2.86-2.77 (m, 2 H), |
| | | | | 2.72-2.66 (m, 1 H), 2.58-2.53 (m, 1 H), 2.35-2.25 (m, |
| | | | | 2 H), 2.18-2.10 (m, 1 H), 2.01-1.92 (m, 3 H), |
| | | | | 1.90-1.84 (m, 2 H), 1.77-1.64 (m, 4 H) |
| I-708 | IV | AR | 836.4 | 12.19 (br s, 1 H), 10.87 (br s, 1 H), 7.81-7.66 (m, 1 H), |
| | | | | 7.57-7.49 (m, 1 H), 7.45-7.36 (m, 1 H), 7.30-7.21 |
| | | | | (m, 2 H), 7.08-6.97 (m, 1 H), 6.81-6.74 (m, 1 H), |
| | | | | 6.66-6.59 (m, 1 H), 6.56-6.47 (m, 1 H), 6.25-6.11 |
| | | | | (m, 2 H), 5.42-5.14 (m, 1 H), 4.42-4.30 (m, 5 H), |
| | | | | 3.85-3.80 (m, 3 H), 3.66-3.56 (m, 2 H), 3.19-2.94 |
| | | | | (m, 8 H), 2.87-2.73 (m, 3 H), 2.59-2.55 (m, 1 H), |
| | | | | 2.36-2.26 (m, 2 H), 2.17-2.11 (m, 1 H), 2.02-1.93 |
| | | | | (m, 3 H), 1.91-1.84 (m, 2 H), 1.78-1.66 (m, 4 H) |
| I-720 | BT | KC | 776.4 | 12.14-12.02 (m, 1 H), 10.89-10.72 (m, 1 H), 7.71 |
| | | | | (dd, J = 2.0, 10.4 Hz, 1 H), 7.61 (d, J = 8.0 Hz, 2 H), |
| | | | | 7.47-7.37 (m, 3 H), 7.10-7.01 (m, 3 H), 6.98 |
| | | | | (d, J = 8.4 Hz, 1 H), 6.85 (d, J = 2.0 Hz, 1 H), 6.20 |
| | | | | (td, J = 2.0, 10.4 Hz, 1 H), 4.49 (d, J = 12.0 Hz, 1 H), |
| | | | | 4.35 (q, J = 6.8 Hz, 2 H), 3.93-3.77 (m, 2 H), 3.49 |
| | | | | (d, J = 11.2 Hz, 2 H), 3.24 (d, J = 13.2 Hz, 1 H), 3.21-3.01 |
| | | | | (m, 6 H), 3.00-2.88 (m, 3 H), 2.85-2.69 (m, 4 H), |
| | | | | 2.68-2.52 (m, 2 H), 2.27-2.15 (m, 1 H), 2.02 (td, J = 4.4, |
| | | | | 8.4 Hz, 1 H), 1.97-1.84 (m, 6 H), 1.76 (d, J = 12.0 Hz, |
| | | | | 1 H), 1.58-1.37 (m, 1 H) |
| I-723^{c,d} | GJ | KC | 905.4 | 12.12-11.98 (m, 1 H), 10.85 (s, 1 H), 7.71 (dd, J = |
| | | | | 2.0, 10.3 Hz, 1 H), 7.61-7.51 (m, 2 H), 7.46-7.35 |
| | | | | (m, 1 H), 7.19-7.08 (m, 2 H), 7.02-6.97 (m, 1 H), |
| | | | | 6.87-6.80 (m, 1 H), 6.64-6.52 (m, 2 H), 6.24-6.13 |
| | | | | (m, 1 H), 5.19-5.07 (m, 1 H), 4.54-4.43 (m, 1 H), |
| | | | | 4.39-4.32 (m, 2 H), 4.30-4.22 (m, 1 H), 3.95 |
| | | | | (br d, J = 8.8 Hz, 2 H), 3.87 (br s, 1 H), 3.82-3.78 (m, 3 H), |
| | | | | 3.71-3.60 (m, 2 H), 3.25 (br s, 3 H), 3.23-3.01 (m, |
| | | | | 10 H), 2.99-2.87 (m, 3 H), 2.86-2.60 (m, 5 H), 2.57 |
| | | | | (br s, 1 H), 2.30-2.14 (m, 2 H), 2.14-2.07 (m, 1 H), |
| | | | | 1.97-1.82 (m, 5 H), 1.80-1.74 (m, 1 H), 1.54-1.39 |
| | | | | (m, 1 H) |
| I-721^{c,d} | FU | KE | 792.3 | 12.20-11.97 (m, 1 H), 11.01 (s, 1 H), 8.15-8.07 (m, |
| | | | | 1 H), 7.70 (d, J = 10.4 Hz, I H), 7.63-7.58 (m, 2 H), |
| | | | | 7.43 (d, J = 8.0 Hz, 2 H), 7.11-7.05 (m, 1 H), |
| | | | | 6.94-6.84 (m, 2 H), 6.54 (dd, J = 2.4, 15.2 Hz, 1 H), |
| | | | | 6.48-6.42 (m, 1 H), 5.86-5.79 (m, 1 H), 4.62 |
| | | | | (q, J = 6.8 Hz, 2 H), 4.53-4.46 (m, 1 H), 4.32-4.24 (m, 1 H), |
| | | | | 3.96-3.88 (m, 1 H), 3.23-3.15 (m, 4 H), 3.13-2.96 |
| | | | | (m, 8 H), 2.78-2.68 (m, 4 H), 2.63-2.59 (m, 1 H), |
| | | | | 2.14-2.07 (m, 1 H), 1.98-1.86 (m, 7 H), 1.84-1.73 |
| | | | | (m, 2 H), 1.58-1.43 (m, 1 H) |
| I-722^{c} | FK | KC | 858.2 | |
| I-738 | LD | AR | 824.3 | 12.35-11.87 (m, 1 H), 11.29-10.68 (m, 1 H), |
| | | | | 7.77-7.67 (m, 1 H), 7.46 (d, J = 2.0 Hz, I H), 7.32 (s, 1 H), |
| | | | | 7.30-7.23 (m, 3 H), 7.21-7.17 (m, 1 H), 6.92-6.80 |
| | | | | (m, 1 H), 6.72-6.65 (m, 1 H), 6.40 (d, J = 2.8 Hz, 1 H), |
| | | | | 6.24-6.11 (m, 2 H), 5.75-5.65 (m, 1 H), 4.40-4.34 |
| | | | | (m, 3 H), 3.68-3.55 (m, 2 H), 3.53-3.43 (m, 2 H), |
| | | | | 3.28-3.01 (m, 6 H), 3.01-2.90 (m, 3 H), 2.90-2.75 |
| | | | | (m, 4 H), 2.73 (d, J = 2.8 Hz, 2 H), 2.68 (s, 3 H), 2.24 |
| | | | | (d, J = 14.0 Hz, 3 H), 2.18 (s, 3 H), 2.06-1.95 (m, 4 H) |
| I-739 | KZ | AR | 855.4 | 12.34-12.03 (m, 1 H), 11.00-10.64 (m, 1 H), |
| | | | | 7.76-7.65 (m, 1 H), 7.45-7.32 (m, 2 H), 7.07-6.97 |
| | | | | (m, 2 H), 6.79 (d, J = 7.2 Hz, 1 H), 6.67-6.59 (m, 1 H), |
| | | | | 6.52 (d, J = 12.8 Hz, 1 H), 6.24-6.10 (m, 2 H), 5.30 |
| | | | | (d, J = 6.8 Hz, 1 H), 4.40-4.29 (m, 5 H), 3.82 (s, 3 H), |
| | | | | 3.68-3.61 (m, 1 H), 3.61-3.55 (m, 3 H), 3.20-3.03 |
| | | | | (m, 5 H), 3.01-2.96 (m, 2 H), 2.91-2.80 (m, 4 H), |
| | | | | 2.57 (d, J = 3.2 Hz, 1 H), 2.33 (s, 1 H), 2.31-2.23 |
| | | | | (m, 1 H), 2.17-2.10 (m, 1 H), 2.00-1.88 (m, 3 H), |
| | | | | 1.84-1.77 (m, 2 H) |
| I-758 | ID | AR | 845.3 | 12.27 (s, 1 H), 10.85 (s, 1 H), 7.75-7.67 (m, 1 H), 7.56 |
| | | | | (d, J = 8.8 Hz, 1 H), 7.51 (d, J = 2.0 Hz, 1 H), 7.43 |
| | | | | (s, 1 H), 7.39 (d, J = 15.2 Hz, 1 H), 7.11-6.99 (m, 1 H), |
| | | | | 6.67 (d, J = 8.0 Hz, 1 H), 6.65 (d, J = 2.8 Hz, 1 H), 6.59 |
| | | | | (d, J = 14.4 Hz, 1 H), 6.23-6.11 (m, 2 H), 5.14 |
| | | | | (d, J = 6.8 Hz, 1 H), 4.41-4.24 (m, 5 H), 3.82 (s, 3 H), |
| | | | | 3.67-3.55 (m, 2 H), 3.44 ( s, 4 H), 3.23-2.93 (m, 12 H), 2.81 |
| | | | | (ddd, J = 5.2, 13.2, 18.0, 1 H), 2.57 (d, J = 3.2 Hz, 1 H), |
| | | | | 2.33 (s, 1 H), 2.27 ( s, 1 H), 2.19-2.08 (m, 1 H), 1.93 |
| | | | | (dq, J = 4.4, 12.8 Hz, 1 H) |
| I-759 | IE | AR | 774.3 | 12.21 (br d, J = 1.6 Hz, I H), 10.84 (s, 1 H), 7.77-7.68 |
| | | | | (m, 1 H), 7.62 (d, J = 8.0 Hz, 2 H), 7.49-7.33 (m, 3 H), |
| | | | | 7.15-6.98 (m, 2 H), 6.88 (d, J = 2.8 Hz, 1 H), |
| | | | | 6.84-6.75 (m, 2 H), 6.24-6.12 (m, 2 H), 4.45-4.27 (m, |
| | | | | 4 H), 3.96-3.81 (m, 3 H), 3.69-3.52 (m, 2 H), |
| | | | | 3.25-2.93 (m, 8 H), 2.87-2.69 (m, 4 H), 2.57-2.52 (m, |
| | | | | 1 H), 2.40-2.24 (m, 2 H), 2.16 (dq, J = 4.0, 12.8 Hz, |
| | | | | 1 H), 2.04-1.87 (m, 3 H), 1.85-1.72 (m, 2 H) |
| I-760^{c} | EX | IF | 786.9 | 13.81 (s, 1 H), 10.87 (s, 1 H), 8.11 (d, J = 12.0 Hz, 1 H), |
| | | | | 8.02 (d, J = 1.6 Hz, 1 H), 7.72-7.66 (m, 1 H), |
| | | | | 7.51-7.44 (m, 1 H), 7.14-7.06 (m, 2 H), 6.70 (d, J = 8.0 Hz, |
| | | | | 1 H), 6.59 (d, J = 14.4 Hz, 1 H), 6.23 ( d, J = 1.2 Hz, |
| | | | | 1 H), 5.15 (d, J = 6.8 Hz, 1 H), 4.62 (q, J = 7.2 Hz, 2 H), |
| | | | | 4.36 (d, J = 18.4 Hz, 2 H), 4.32-4.25 (m, 1 H), 3.82 (s, |
| | | | | 3 H), 3.63 (td, J = 5.6, 20.0 Hz, 2 H), 3.27 (s, 3 H), 3.11 |
| | | | | (d, J = 3.2 Hz, 6 H), 2.85-2.77 (m, 1 H), 2.56 (d, J = |
| | | | | 2.8 Hz, 1 H), 2.39-2.26 (m, 3 H), 2.16-2.10 (m, 1 H), |
| | | | | 1.98-1.90 (m, 1 H) |
| I-761^{c,d} | II | AK | 838.4 | 12.23 (d, J = 7.2 Hz, 1 H), 10.88 (s, 1 H), 8.19-8.03 |
| | | | | (m, 1 H), 7.72-7.63 (m, 3 H), 7.51 (d, J = 8.4 Hz, 2 H), |
| | | | | 7.13-7.01 (m, 1 H), 6.89 (s, 1 H), 6.71 (d, J = 8.0 Hz, |
| | | | | 1 H), 6.64-6.55 (m, 1 H), 6.18 (d, J = 7.2 Hz, 1 H), |
| | | | | 5.18 (d, J = 6.4 Hz, 1 H), 5.02-4.82 (m, 1 H), |
| | | | | 4.67-4.59 (m, 2 H), 4.45-4.34 (m, 2 H), 4.30 (dd, J = 6.0, |
| | | | | 12.4 Hz, 1 H), 3.86-3.80 (m, 3 H), 3.68-3.58 (m, |
| | | | | 3 H), 3.40-3.38 (m, 6 H), 3.21 (s, 1 H), 3.11 (d, J = 6.4 |
| | | | | Hz, 2 H), 2.91-2.76 (m, 4 H), 2.55 (s, 2 H), |
| | | | | 2.37-2.27 (m, 2 H), 2.17-2.09 (m, 1 H), 1.97-1.92 (m, |
| | | | | 2 H) |
| I-746 | JE | LN | 810.2 | 12.30-12.24 (m, 1 H), 10.83 (s, 1 H), 7.75-7.69 (m, |
| | | | | 1 H), 7.45-7.33 (m, 3 H), 7.12-7.07 (m, 2 H), |
| | | | | 7.06-7.04 (m, 1 H), 7.02-6.97 (m, 1 H), 6.70-6.65 (m, |
| | | | | 1 H), 6.24-6.13 (m, 2 H), 4.41-4.35 (m, 3 H), 4.31 |
| | | | | (br s, 1 H), 3.83 (dd, J = 4.8, 11.6 Hz, 1 H), 3.65 |
| | | | | (br t, J = 5.6 Hz, 1 H), 3.59 (br t, J = 5.6 Hz, 1 H), 3.51 |
| | | | | (br d, J = 11.2 Hz, 2 H), 3.24-3.13 (m, 3 H), 3.11-3.04 |
| | | | | (m, 3 H), 3.02-2.94 (m, 3 H), 2.90-2.82 (m, 2 H), 2.66 |
| | | | | (br d, J = 12.0 Hz, 1 H), 2.34 (br d, J = 2.0 Hz, 1 H), |
| | | | | 2.27 (br d, J = 4.0 Hz, 1 H), 2.22 (br dd, J = 3.6, 12.4 |
| | | | | Hz, 1 H), 2.08-1.88 (m, 6 H) |
| I-747 | JE | ME | 825.4 | 12.39-12.10 (m, 1 H), 11.03-10.71 (m, 1 H), 7.83 |
| | | | | (d, J = 9.6 Hz, 1 H), 7.62-7.40 (m, 2 H), 7.09 |
| | | | | (d, J = 5.6 Hz, 3 H), 6.85-6.51 (m, 3 H), 6.39-6.22 (m, 2 H), |
| | | | | 6.18-6.08 (m, 1 H), 4.51-4.38 (m, 5 H), 3.78-3.66 |
| | | | | (m, 4 H), 3.32-3.15 (m, 6 H), 3.11-3.06 (m, 2 H), |
| | | | | 2.98-2.92 (m, 2 H), 2.88-2.80 (m, 1 H), 2.73-2.66 |
| | | | | (m, 1 H), 2.50-2.34 (m, 3 H), 2.25-2.15 (m, 1 H), |
| | | | | 2.05-1.88 (m, 5 H) |
| I-740 | JE | LE | 792.4 | 12.30-12.20 (m, 1 H), 10.88-10.80 (m, 1 H), |
| | | | | 7.76-7.69 (m, 1 H), 7.54-7.38 (m, 4 H), 7.15-7.04 (m, |
| | | | | 3 H), 7.01-6.96 (m, 1 H), 6.92-6.87 (m, 1 H), |
| | | | | 6.24-6.15 (m, 2 H), 4.43-4.31 (m, 4 H), 3.86-3.79 (m, |
| | | | | 1 H), 3.67-3.57 (m, 2 H), 3.54-3.47 (m, 2 H), |
| | | | | 3.29-3.05 (m, 6 H), 3.03 (br s, 4 H), 2.88-2.80 (m, 2 H), |
| | | | | 2.72-2.64 (m, 1 H), 2.34 (br s, 1 H), 2.31-2.16 (m, |
| | | | | 2 H), 2.06-2.00 (m, 1 H), 1.93 (br s, 4 H) |
| I-748 | LR | JF | 794.3 | 12.14-11.98 (m, 1 H), 10.91-10.84 (m, 1 H), |
| | | | | 7.74-7.64 (m, 1 H), 7.62-7.56 (m, 2 H), 7.44-7.35 (m, |
| | | | | 3 H), 7.08-7.00 (m, I H), 6.86-6.82 (m, 1 H), |
| | | | | 6.72-6.67 (m, 2 H), 6.25-6.14 (m, 1 H), 4.54-4.42 (m, |
| | | | | 1 H), 4.38-4.31 (m, 2 H), 4.12-4.02 (m, 1 H), |
| | | | | 3.98-3.86 (m, 3 H), 3.16-2.98 (m, 6 H), 2.74 (br s, 8 H), |
| | | | | 2.69-2.60 (m, 2 H), 2.37-2.23 (m, 2 H), 2.12-1.97 |
| | | | | (m, 2 H), 1.93-1.86 (m, 4 H), 1.79-1.71 (m, 3 H), |
| | | | | 1.57-1.14 (m, 2 H) |
| I-749 | LQ | LV | 812.2 | 12.23-12.01 (m, 1 H), 10.93-10.70 (m, 1 H), |
| | | | | 7.75-7.67 (m, 1 H), 7.57-7.35 (m, 4 H), 7.19-7.10 (m, |
| | | | | 1 H), 7.03-6.93 (m, 2 H), 6.91-6.83 (m, 1 H), 6.21 |
| | | | | (br d, J = 10.8 Hz, 1 H), 4.56-4.44 (m, 1 H), |
| | | | | 4.40-4.30 (m, 2 H), 3.97-3.76 (m, 2 H), 3.31-3.17 |
| | | | | (m, 7 H), 3.16-2.99 (m, 6 H), 2.97-2.71 (m, 3 H), |
| | | | | 2.70-2.62 (m, 1 H), 2.55 (br d, J = 3.2 Hz, 1 H), 2.32-2.21 |
| | | | | (m, 1 H), 2.00-1.85 (m, 6 H), 1.83-1.73 (m, 1 H), |
| | | | | 1.60-1.40 (m, 1 H) |
| I-750 | LG | N | 818.4 | 11.97 (br d, J = 8.0 Hz, 1 H), 10.71 (br s, 1 H), |
| | | | | 7.65-7.55 (m, 1 H), 7.34-7.17 (m, 2 H), 6.84-6.73 |
| | | | | (m, 1 H), 6.50-6.38 (m, 4 H), 6.33 (d, J = 8.4 Hz, 1 H), |
| | | | | 6.11-6.03 (m, 1 H), 6.02-5.95 (m, 1 H), 4.83 |
| | | | | (d, J = 6.4 Hz, 1 H), 4.27-4.21 (m, 3 H), 4.20-4.04 (m, 2 H), |
| | | | | 3.66 (s, 3 H), 3.55-3.38 (m, 4 H), 3.29-3.23 (m, 2 H), |
| | | | | 3.18-3.12 (m, 3 H), 3.08-2.81 (m, 12 H), 2.68 (ddd, |
| | | | | J = 5.2, 13.2, 18.0 Hz, 1 H), 2.26-2.10 (m, 2 H), |
| | | | | 2.07-1.98 (m, 1 H), 1.83-1.70 (m, 1 H) |
| I-752^{c,d} | LO | KD | 893.4 | 12.14-12.04 (m, 1 H), 11.10-11.02 (m, 1 H), 7.71 |
| | | | | (dd, J = 2.0, 9.6 Hz, 1 H), 7.44-7.37 (m, 2 H), |
| | | | | 7.32-7.25 (m, 2 H), 7.18 (d, J = 8.4 Hz, 1 H), 6.96 |
| | | | | (d, J = 6.8 Hz, 2 H), 6.49 (s, 1 H), 6.23-6.16 (m, 1 H), |
| | | | | 5.78-5.67 (m, 1 H), 4.48 (d, J = 12.8 Hz, 1 H), 4.35 |
| | | | | (q, J = 6.4 Hz, 2 H), 3.87 (d, J = 12.0 Hz, 1 H), 3.23 (s, 2 H), |
| | | | | 3.17 (d, J = 4.0 Hz, 7 H), 3.10 (d, J = 6.0 Hz, 1 H), |
| | | | | 3.06-3.02 (m, 2 H), 2.99 (d, J = 8.0 Hz, 2 H), 2.92 (s, 1 H), |
| | | | | 2.88 (d, J = 6.0 Hz, 1 H), 2.86-2.81 (m, 1 H), |
| | | | | 2.80-2.75 (m, 1 H), 2.72 (d, J = 7.2 Hz, 6 H), 2.69-2.68 |
| | | | | (m, 1 H), 2.66 (s, 3 H), 2.22 (dd, J = 5.2, 9.6 Hz, 1 H), |
| | | | | 1.93-1.81 (m, 2 H), 1.75 (d, z = 11.6 Hz, I H), 1.58-1.14 |
| | | | | (m, 2 H) |
| I-753 | LR | LT | 794.2 | 12.09 (br s, 1 H), 10.87 (br s, 1 H), 7.72 (dd, J = 2.0, |
| | | | | 10.8 Hz, 1 H), 7.61 (d, J = 8.0 Hz, 2 H), 7.48-7.38 |
| | | | | (m, 3 H), 7.13-7.04 (m, 1 H), 7.03-6.94 (m, 2 H), 6.86 |
| | | | | (d, J = 2.0 Hz, 1 H), 6.20 (td, J = 2.0, 10.8 Hz, 1 H), |
| | | | | 4.50 (br d, J = 10.8 Hz, 1 H), 4.36 (q, J = 7.2 Hz, 2 H), |
| | | | | 3.97-3.80 (m, 2 H), 3.30-3.15 (m, 7 H), 3.13-2.89 |
| | | | | (m, 7 H), 2.84-2.62 (m, 3 H), 2.55 (t, J = 2.0 Hz, 1 H), |
| | | | | 2.32-2.21 (m, 1 H), 2.05-1.98 (m, 1 H), 1.95-1.75 |
| | | | | (m, 7 H), 1.57-1.37 (m, 1 H) |
| I-741 | LF | KD | 877.3 | 12.15-12.04 (m, 1 H), 11.06 (s, 1 H), 7.71 |
| | | | | (dd, J = 2.0, 10.0 Hz, 1 H), 7.44-7.25 (m, 4 H), 7.04-6.94 |
| | | | | (m, 3 H), 6.61 (s, 1 H), 6.23-6.16 (m, 1 H), 5.73 |
| | | | | (dd, J = 4.8, 11.6 Hz, 1 H), 4.53-4.43 (m, 1 H), 4.39-4.31 |
| | | | | (m, 2 H), 3.95-3.77 (m, 1 H), 3.24-3.11 (m, 10 H), |
| | | | | 3.09-2.98 (m, 4 H), 2.88-2.88 (m, 1 H), 2.95-2.84 |
| | | | | (m, 3 H), 2.75-2.64 (m, 12 H), 2.27-2.17 (m, 1 H), |
| | | | | 1.93-1.72 (m, 3 H), 1.53-1.53 (m, 1 H), 1.58-1.37 |
| | | | | (m, 1 H) |
| I-754^{c} | LU | KD | 877.5 | 12.1 (br s, 1 H), 11.1 (s, I H), 7.71 (dd, J = 1.6, 10.4 |
| | | | | Hz, 1 H), 7.46-7.36 (m, 2 H), 7.32-7.21 (m, 2 H), |
| | | | | 7.07-6.92 (m, 3 H), 6.63 (br s, 1 H), 6.20 (br d, |
| | | | | J = 10.4 Hz, 1 H), 5.79-5.66 (m, 1 H), 4.48 (br d, J = 12.8 |
| | | | | Hz, 1 H), 4.41-4.29 (m, 2 H), 3.94-3.82 (m, 1 H), |
| | | | | 3.25-3.11 (m, 11 H), 3.10-2.96 (m, 5 H), 2.96-2.79 |
| | | | | (m, 4 H), 2.78-2.68 (m, 7 H), 2.66 (s, 3 H), 2.29-2.18 |
| | | | | (m, 1 H), 1.97-1.83 (m, 2 H), 1.78-1.69 (m, 1 H), |
| | | | | 1.57-1.37 (m, 1 H) |
| I-755 | MF | KD | 794.3 | 12.23-11.98 (m, 1 H), 11.06-10.74 (m, 1 H), |
| | | | | 7.75-7.67 (m, 1 H), 7.62-7.58 (m, 2 H), 7.46-7.39 |
| | | | | (m, 3 H), 7.18-7.11 (m, 1 H), 7.09-7.04 (m, 1 H), |
| | | | | 6.96-6.89 (m, 1 H), 6.88-6.84 (m, 1 H), 6.26-6.15 |
| | | | | (m, 1 H), 4.53-4.46 (m, 1 H), 4.38-4.32 (m, 2 H), |
| | | | | 4.01-3.95 (m, 1 H), 3.92-3.85 (m, 1 H), 3.57-3.51 |
| | | | | (m, 2 H), 3.19 (br s, 6 H), 2.95-2.88 (m, 2 H), 2.71 |
| | | | | (br s, 6 H), 2.33 (br s, 2 H), 2.02-1.84 (m, 8 H), |
| | | | | 1.79-1.73 (m, 1 H), 1.54-1.42 (m, 1 H) |
| I-756 | LX | KD | 848.3 | 12.24-12.03 (m, 1 H), 11.21-10.86 (m, 1 H), |
| | | | | 7.80-7.61 (m, 1 H), 7.45-7.35 (m, 3 H), 7.30-7.23 |
| | | | | (m, 1 H), 7.21-7.17 (m, 1 H), 7.12-7.08 (m, 1 H), |
| | | | | 6.71-6.64 (m, 2 H), 6.22-6.16 (m, 1 H), 5.74-5.59 |
| | | | | (m, 1 H), 4.53-4.44 (m, 1 H), 4.39-4.31 (m, 2 H), |
| | | | | 3.95-3.85 (m, 1 H), 3.53-3.44 (m, 2 H), 3.19-3.00 |
| | | | | (m, 8 H), 2.96 (br s, 6 H), 2.79-2.70 (m, 2 H), 2.70-2.66 |
| | | | | (m, 4 H), 2.24-2.18 (m, 1 H), 2.12-1.99 (m, 4 H), |
| | | | | 1.94-1.82 (m, 2 H), 1.80-1.72 (m, 1 H), 1.58-1.43 |
| | | | | (m, 1 H) |
| I-1008^{d} | OK | AR | 871.3 | 12.53-11.83 (m, 1 H), 11.19-10.47 (m, 1 H), |
| | | | | 7.76-7.66 (m, 1 H), 7.44-7.36 (m, 1 H), 7.34-7.28 |
| | | | | (m, 1 H), 7.21 (d, J = 8.0 Hz, I H), 7.00-6.89 (m, 1 H), |
| | | | | 6.79 (d, J = 7.2 Hz, 1 H), 6.56-6.45 (m, 2 H), |
| | | | | 6.23-6.09 (m, 2 H), 5.30 (d, J = 7.2 Hz, 1 H), 4.39-4.28 |
| | | | | (m, 5 H), 3.82 (s, 3 H), 3.66-3.54 (m, 4 H), 3.24-3.00 |
| | | | | (m, 6 H), 2.99-2.94 (m, 2 H), 2.91-2.80 (m, 4 H), |
| | | | | 2.58-2.52 (m, 1 H), 2.35-2.24 (m, 2 H), 2.18-2.10 |
| | | | | (m, 1 H), 2.01-1.88 (m, 3 H), 1.86-1.76 (m, 2 H) |
| I-1010 | OL | AR | 790.2 | 12.22-12.14 (m, 1 H), 10.93 (br s, 1 H), 7.72 |
| | | | | (dd, J = 1.6, 11.2 Hz, 1 H), 7.62 (br d, J = 8.0 Hz, 2 H), |
| | | | | 7.45-7.37 (m, 3 H), 7.07-7.00 (m, 2 H), 6.95-6.90 |
| | | | | (m, 1 H), 6.89-6.85 (m, I H), 6.83-6.78 (m, 1 H), |
| | | | | 6.22-6.14 (m, 2 H), 5.14 (dd, J = 5.2, 10.8 Hz, 1 H), |
| | | | | 4.39-4.30 (m, 4 H), 3.66-3.56 (m, 2 H), 3.12-2.93 |
| | | | | (m, 6 H), 2.81-2.66 (m, 5 H), 2.65-2.54 (m, 2 H), 2.22 |
| | | | | (br d, J = 5.2 Hz, 4 H), 2.20-2.06 (m, 2 H), 1.94-1.84 |
| | | | | (m, 4 H) |
| I-1011 | OL | AK | 791.2 | 12.18 (br d, J = 7.2 Hz, 1 H), 10.93 (s, 1 H), 8.14-8.08 |
| | | | | (m, 1 H), 7.71-7.66 (m, 1 H), 7.62 (br d, J = 8.0 Hz, |
| | | | | 2 H), 7.43 (d, J = 8.4 Hz, 2 H), 7.09-7.04 (m, 1 H), |
| | | | | 7.03-6.99 (m, 1 H), 6.93 (dd, J = 2.8, 14.4 Hz, 1 H), |
| | | | | 6.89-6.85 (m, 1 H), 6.80 (dd, J = 1.6, 8.8 Hz, 1 H), |
| | | | | 6.20-6.13 (m, 1 H), 5.14 (dd, J = 5.2, 10.8 Hz, 1 H), |
| | | | | 4.66-4.58 (m, 2 H), 4.41-4.30 (m, 2 H), 3.68-3.59 |
| | | | | (m, 2 H), 3.17-3.07 (m, 6 H), 2.82-2.66 (m, 5 H), |
| | | | | 2.64-2.55 (m, 2 H), 2.40-2.25 (m, 4 H), 2.22-2.10 |
| | | | | (m, 2 H), 1.94-1.85 (m, 4 H) |
| I-1012 | MF | AR | 792.3 | 12.23 (s, 1 H), 10.90 (s, 1 H), 7.80-7.68 (m, 1 H), |
| | | | | 7.66-7.60 (m, 2 H), 7.49-7.37 (m, 3 H), 7.20-7.13 |
| | | | | (m, 1 H), 6.97-6.87, (m, 1 H), 6.71-6.63, (m, 1 H), |
| | | | | 6.43-6.40, (m, 1 H), 6.22-6.13, (m, 2 H), 4.40-4.30, |
| | | | | (m, 4 H), 4.10-4.04, (m, 1 H), 3.90-3.74, (m, 6 H), |
| | | | | 3.45-3.30, (m, 2 H), 3.12-3.04, (m, 2 H), 2.86-2.81, |
| | | | | (m, 2 H), 2.62-2.54, (m, 1 H), 2.36-2.32, (m, 1 H), |
| | | | | 2.16-2.01, (m, 3 H), 1.92-1.76, (m, 5 H) |
| I-1013 | MO | AR | 893.5 | 12.19 (br d, J = 8.4 Hz, 1 H), 10.84 (s, 1 H), 7.74-7.68 |
| | | | | (m, 1 H), 7.43 (s, 3 H), 7.05-6.96 (m, 4 H), 6.63 (br s, |
| | | | | 1 H), 6.21-6.12 (m, 2 H), 4.36 (q, J = 7.2 Hz, 4 H), |
| | | | | 4.30-4.25 (m, 1 H), 3.89-3.84 (m, 1 H), 3.66-3.53 |
| | | | | (m, 4 H), 3.20-3.11 (m, 3 H), 3.06-2.95 (m, 6 H), |
| | | | | 2.79-2.72 (m, 3 H), 2.68-2.62 (m, 1 H), 2.36-2.20 |
| | | | | (m, 6 H), 2.05-1.99 (m, I H), 1.91-1.86 (m, 2 H), |
| | | | | 1.78-1.69 (m, 4 H), 1.68-1.58 (m, 3 H) |
| I-1014 | MY | AK | 812.2 | 12.24-12.15 (m, 1 H), 10.82 (s, 1 H), 8.14-8.08 |
| | | | | (m, 1 H), 7.71-7.66 (m, 1 H), 7.44-7.37 (m, 1 H), |
| | | | | 7.10-7.05 (m, 3 H), 7.04-6.97 (m, 2 H), 6.66-6.61 |
| | | | | (m, 1 H), 6.19-6.11 (m, 1 H), 4.66-4.58 (m, 2 H), |
| | | | | 4.39-4.30 (m, 2 H), 3.86-3.80 (m, 1 H), 3.68-3.57 |
| | | | | (m, 2 H), 3.29-3.26 (m, 4 H), 3.20 (br d, J = 2.4 Hz, 6 H), |
| | | | | 3.13-3.00 (m, 6 H), 2.72-2.62 (m, 1 H), 2.34 |
| | | | | (br dd, J = 2.4, 5.6 Hz, 2 H), 2.30-2.17 (m, 2 H), |
| | | | | 2.06-1.98 (m, 1 H) |
| I-1015 | BT | AK | 775.4 | 12.19 (br d, J = 4.0 Hz, 1 H), 10.83 (s, 1 H), 8.21-8.06 |
| | | | | (m, 1 H), 7.69 (d, J = 11.6 Hz, 1 H), 7.63 (br d, J = 8.0 |
| | | | | Hz, 2 H), 7.44 (d, J = 8.0 Hz, 2 H), 7.18-6.95 (m, 4 H), |
| | | | | 6.88 (br s, 1 H), 6.17 (br s, 1 H), 4.73-4.55 (m, 2 H), |
| | | | | 4.48-4.27 (m, 2 H), 3.82 (dd, J = 4.8, 11.6 Hz, 1 H), |
| | | | | 3.72-3.57 (m, 2 H), 3.50 (br d, J = 11.2 Hz, 2 H), |
| | | | | 3.27-2.92 (m, 8 H), 2.87-2.78 (m, 2 H), 2.76-2.61 (m, |
| | | | | 2 H), 2.53 (br s, 1 H), 2.41-2.27 (m, 2 H), 2.26-2.16 |
| | | | | (m, 1 H), 2.11-2.01 (m, 1 H), 2.00-1.79 (m, 4 H) |
| I-1016 | BT | AK | 775.4 | 12.19 (br d, J = 4.0 Hz, 1 H), 10.83 (s, 1 H), 8.21-8.06 |
| | | | | (m, 1 H), 7.69 (d, J = 11.6 Hz, 1 H), 7.63 (br d, J = 8.0 |
| | | | | Hz, 2 H), 7.44 (d, J = 8.0 Hz, 2 H), 7.18-6.95 (m, 4 H), |
| | | | | 6.88 (br s, 1 H), 6.17 (br s, 1 H), 4.73-4.55 (m, 2 H), |
| | | | | 4.48-4.27 (m, 2 H), 3.82 (dd, J = 4.8, 11.6 Hz, 1 H), |
| | | | | 3.72-3.57 (m, 2 H), 3.50 (br d, J = 11.2 Hz, 2 H), |
| | | | | 3.27-2.92 (m, 8 H), 2.87-2.78 (m, 2 H), 2.76-2.61 |
| | | | | (m, 2 H), 2.53 (br s, 1 H), 2.41-2.27 (m, 2 H), 2.26-2.16 |
| | | | | (m, 1 H), 2.11-2.01 (m, 1 H), 2.00-1.79 (m, 4 H) |
| I-1017 | NP | AK | 812.4 | 12.36-12.10 (m, 1 H), 10.97-10.67 (m, 1 H), 8.17 (br |
| | | | | d, J = 12.0 Hz, 1 H), 7.74 (s, 1 H), 7.38-7.26 (m, 1 H), |
| | | | | 7.14-6.99 (m, 5 H), 6.67 (br d, J = 2.0 Hz, 1 H), 6.21- |
| | | | | 6.08 (m, 1 H), 4.67-4.60 (m, 2 H), 4.40-4.29 (m, |
| | | | | 2 H), 3.87-3.77 (m, 1 H), 3.69-3.58 (m, 2 H), 3.30- |
| | | | | 3.26 (m, 4 H), 3.25-3.04 (m, 12 H), 2.70-2.62 (m, |
| | | | | 1 H), 2.44-2.13 (m, 4 H), 2.08-2.00 (m, 1 H) |
| I-1018 | MU | AR | 906.6 | 1.88-2.02 (m, 4 H) 2.03-2.17 (m, 2 H) 2.26-2.38 |
| | | | | (m, 2 H) 2.58-2.90 (m, 2 H) 2.58-2.85 (m, 1 H) |
| | | | | 2.96-3.01 (m, 2 H) 3.03-3.11 (m, 6 H) 3.12-3.27 |
| | | | | (m, 4 H) 3.44-3.71 (m, 1 H) 3.54-3.67 (m, 2 H) |
| | | | | 3.99-4.49 (m, 1 H) 4.27-4.43 (m, 4 H) 4.66-4.74 |
| | | | | (m, 1 H) 6.13-6.22 (m, 3 H) 6.97-7.07 (m, 1 H) |
| | | | | 7.11-7.21 (m, 1 H) 7.36-7.46 (m, 3 H) 7.47-7.57 |
| | | | | (m, 1 H) 7.69-7.82 (m, 1 H) 8.46 (br d, J = 7.2 Hz, 1 |
| | | | | H) 10.79-10.98 (m, 1 H) 12.04-12.29 (m, 1 H) |
| I-1019 | JE | MM | 789.4 | 12.26-12.14 (m, 1 H), 10.90 (s, 1 H), 8.16-8.09 (m, |
| | | | | 1 H), 7.76-7.69 (m, 1 H), 7.67-7.60 (m, 2 H), |
| | | | | 7.50-7.36 (m, 3 H), 7.09-7.00 (m, 1 H), 6.91-6.86 |
| | | | | (m, 1 H), 6.23-6.14 (m, 2 H), 4.41-4.31 (m, 4 H), |
| | | | | 4.24-4.15 (m, 1 H), 3.68-3.57 (m, 2 H), 3.31-3.27 |
| | | | | (m, 2 H), 3.26-3.03 (m, 6 H), 3.03-2.86 (m, 5 H), |
| | | | | 2.85-2.70 (m, 2 H), 2.70-2.58 (m, 1 H), 2.56 (br d, J = 4.8 |
| | | | | Hz, 1 H), 2.34 (br s, 1 H), 2.26 (s, 3 H), 2.13-2.06 |
| | | | | (m, 1 H), 2.00-1.87 (m, 4 H) |
| I-1020 | MQ | AR | 811.2 | 12.24 (d, J = 8.4 Hz, 1 H), 11.03-10.79 (m, 1 H), 8.32 |
| | | | | (s, 1 H), 7.76-7.68 (m, 1 H), 7.67-7.60 (m, 1 H), |
| | | | | 7.46-7.36 (m, 1 H), 7.35-7.27 (m, 1 H), 7.07-6.95 |
| | | | | (m, 2 H), 6.68 (s, 1 H), 6.25-6.08 (m, 2 H), 4.43-4.34 |
| | | | | (m, 3 H), 4.32 (s, 1 H), 4.06-3.97 (m, 1 H), 3.69-3.55 |
| | | | | (m, 4 H), 3.29-3.07 (m, 6 H), 3.07-3.03 (m, 1 H), |
| | | | | 3.01-2.92 (m, 4 H), 2.72 (s, 1 H), 2.61-2.54 (m, 1 H), |
| | | | | 2.40-2.26 (m, 3 H), 2.11-1.98 (m, 3 H), 1.92 |
| | | | | (d, J = 11.6 Hz, 2 H) |
| I-1021^{e} | MX | AR | 924.2 | 12.46-12.06 (m, 1 H), 11.19-10.62 (m, 1 H), 8.48 (d, |
| | | | | J = 6.8 Hz, 1 H), 7.89-7.63 (m, 1 H), 7.58-7.48 (m, |
| | | | | 1 H), 7.46-7.35 (m, 1 H), 7.34-7.22 (m, 1 H), |
| | | | | 7.07-6.89 (m, 2 H), 6.71-6.57 (m, 1 H), 6.24-6.09 |
| | | | | (m, 3 H), 4.81-4.60 (m, 1 H), 4.40-4.29 (m, 4 H), 3.92 |
| | | | | (d, J = 7.6 Hz, 6 H), 3.67-3.57 (m, 2 H), 3.30 (s, 2 H), |
| | | | | 3.20-3.04 (m, 9 H), 3.03-2.93 (m, 3 H), 2.83-2.71 |
| | | | | (m, 1 H), 2.36-2.26 (m, 2 H), 2.16-2.06 (m, 2 H), |
| | | | | 2.03-1.95 (m, 4 H) |
| I-1022 | JE | NT | 775.4 | 1.86-2.00 (m, 4 H) 2.10-2.17 (m, 1 H) 2.24-2.37 |
| | | | | (m, 3 H) 2.59-2.63 (m, 1 H) 2.76-2.81 (m, 1 H) |
| | | | | 2.93-3.01 (m, 4 H) 3.05-3.23 (m, 6 H) 3.54-3.60 |
| | | | | (m, 4 H) 3.63-3.67 (m, 1 H) 3.98 (br dd, J = 9.6, 5.2 |
| | | | | Hz, 1 H) 4.31-4.41 (m, 4 H) 6.12-6.24 (m, 2 H) |
| | | | | 6.88 (br s, 1 H) 6.99-7.09 (m, 1 H) 7.28 (d, J = 13.2 |
| | | | | Hz, 1 H) 7.44 (br d, J = 7.2 Hz, 3 H) 7.63 (br d, J = 8.0 |
| | | | | Hz, 2 H) 7.72 (dd, J = 10.8, 1.6 Hz, 1 H) 8.30 (d, |
| | | | | J = 11.2 Hz, 1 H) 10.13-10.37 (m, 1 H) 10.85 (br s, 1 |
| | | | | H) 12.07-12.31 (m, 1 H). |
| I-1023 | JE | NX | 895.4 | 12.22 (br d, J = 8.4 Hz, 1 H), 10.86 (s, 1 H), 7.89 (s, |
| | | | | 1 H), 7.72 (dd, J = 1.6, 11.6 Hz, 1 H), 7.50-7.35 (m, |
| | | | | 2 H), 7.33-7.22 (m, 1 H), 7.06-6.89 (m, 2 H), 6.66 |
| | | | | (br s, 1 H), 6.28-6.07 (m, 2 H), 4.40-4.27 (m, 4 H), |
| | | | | 4.02 (br d, J = 11.2 Hz, 2 H), 3.86 (br dd, J = 4.4, 12.4 |
| | | | | Hz, 1 H), 3.70-3.52 (m, 3 H), 3.14 (br s, 6 H), 3.06 |
| | | | | (br dd, J = 2.8, 6.8 Hz, 2 H), 3.01-2.96 (m, 2 H), 2.87 |
| | | | | (br t, J = 12.0 Hz, 2 H), 2.71 (br s, 6 H), 2.56 (br d, J = 4.0 |
| | | | | Hz, 1 H), 2.41-2.13 (m, 4 H), 2.05-1.96 (m, 1 H), |
| | | | | 1.94-1.86 (m, 2 H), 1.55 (br d, J = 9.2 Hz, 2 H) |
| I-1024^{d} | MZ | AK | 811.1 | 12.25-12.15 (m, 1 H), 10.88-10.83 (m, 1 H), |
| | | | | 8.15-8.06 (m, 1 H), 7.71-7.65 (m, 1 H), 7.41-7.34 |
| | | | | (m, 2 H), 6.97 (s, 4 H), 6.67-6.61 (m, 1 H), 6.19-6.12 |
| | | | | (m, 1 H), 4.65-4.59 (m, 2 H), 4.39-4.30 (m, 2 H), |
| | | | | 3.92-3.85 (m, 1 H), 3.67-3.58 (m, 4 H), 3.20-3.03 |
| | | | | (m, 8 H), 2.99 (br d, J = 6.0 Hz, 4 H), 2.71-2.63 (m, 1 H), |
| | | | | 2.35 (br s, 1 H), 2.30-2.21 (m, 2 H), 2.07-2.00 |
| | | | | (m, 1 H), 1.96-1.86 (m, 4 H) |
| I-1025^{d, f} | NB | AR | 790.2 | 12.32-12.08 (m, 1 H), 10.85 (s, 1 H), 7.77-7.67 (m, |
| | | | | 1 H), 7.64 (d, J = 8.0 Hz, 2 H), 7.49-7.36 (m, 3 H), |
| | | | | 7.32 (s, 1 H), 7.21-7.15 (m, 2 H), 7.09-7.00 (m, 1 H), |
| | | | | 6.88 (s, 1 H), 6.27-6.08 (m, 2 H), 4.41-4.30 (m, 4 H), |
| | | | | 3.89-3.82 (m, 1 H), 3.68-3.56 (m, 2 H), 3.44 |
| | | | | (d, J = 10.8 Hz, 2 H), 3.27-3.02 (m, 6 H), 3.01-2.95 |
| | | | | (m, 2 H), 2.87-2.79 (m, 2 H), 2.77-2.62 (m, 2 H), 2.56 |
| | | | | (d, J = 3.1 Hz, 1 H), 2.32 (s, 2 H), 2.26-2.19 (m, 1 H), |
| | | | | 2.09-1.96 (m, 2 H), 1.96-1.87 (m, 3 H) |
| I-1026 | NB | AK | 791.5 | 12.20 (d, J = 6.1 Hz, 1 H), 10.84 (s, 1 H), 8.12 |
| | | | | (d, J = 11.3 Hz, 1 H), 7.72 (d, J = 11.9 Hz, 1 H), 7.64 |
| | | | | (d, J = 8.0 Hz, 2 H), 7.44 (d, J = 8.3 Hz, 2 H), 7.32 (s, 1 H), |
| | | | | 7.16 (s, 2 H), 7.10-7.00 (m, 1 H), 6.88 (s, 1 H), 6.16 (s, |
| | | | | 1 H), 4.69-4.56 (m, 2 H), 4.43-4.28 (m, 2 H), |
| | | | | 3.91-3.77 (m, 1 H), 3.71-3.57 (m, 2 H), 3.44 (d, J = 10.1 |
| | | | | Hz, 2 H), 3.27-3.18 (m, 2 H), 3.18-2.98 (m, 6 H), |
| | | | | 2.87-2.71 (m, 3 H), 2.70-2.61 (m, 1 H), 2.56 |
| | | | | (d, J = 3.0 Hz, 1 H), 2.36 (s, 1 H), 2.33-2.16 (m, 2 H), |
| | | | | 2.08-1.99 (m, 1 H), 1.99-1.81 (m, 4 H) |
| I-1027 | NO | AK | 793.2 | 12.10 (s, 1 H), 10.97-10.73 (m, 1 H), 8.19-8.01 |
| | | | | (m, 1 H), 7.80-7.61 (m, 1 H), 7.56-7.43 (m, 3 H), |
| | | | | 7.14-6.87 (m, 5 H), 6.29-6.00 (m, 1 H), 4.71-4.56 |
| | | | | (m, 2 H), 4.45-4.30 (m, 2 H), 3.90 (s, 1 H), 3.69-3.56 |
| | | | | (m, 2 H), 3.50 (br d, J = 11.2 Hz, 2 H), 3.22-3.01 (m, 9 H), |
| | | | | 2.90-2.80 (m, 2 H), 2.71-2.64 (m, 1 H), 2.40-2.28 |
| | | | | (m, 3 H), 2.25-2.16 (m, 1 H), 2.05-1.87 (m, 5 H) |
| I-1028 | NE | AK | 811.7 | 12.86-11.49 (m, 1 H), 10.93-10.70 (m, 1 H), |
| | | | | 8.14-8.08 (m, 1 H), 7.72-7.65 (m, 1 H), 7.46-7.38 |
| | | | | (m, 2 H), 7.11-7.01 (m, 3 H), 7.01-6.96 (m, 1 H), |
| | | | | 6.66-6.62 (m, 1 H), 6.20-6.13 (m, 1 H), 4.65-4.59 |
| | | | | (m, 2 H), 4.40-4.30 (m, 2 H), 3.82 (dd, J = 4.8, 11.6 Hz, |
| | | | | 1 H), 3.67-3.57 (m, 2 H), 3.49 (d, J = 11.2 Hz, 2 H), |
| | | | | 3.24-3.13 (m, 3 H), 3.12-3.00 (m, 6 H), 2.88-2.80 |
| | | | | (m, 2 H), 2.69-2.61 (m, 1 H), 2.52 (s, 1 H), 2.37-2.26 |
| | | | | (m, 2 H), 2.24-2.15 (m, 1 H), 2.05-1.99 (m, 1 H), |
| | | | | 1.98-1.87 (m, 4 H) |
| I-1029^{c,d} | NZ | AR | 903.4 | 12.12 (br d, J = 6.4 Hz, 1 H), 10.39 (s, 1 H), 7.77-7.67 |
| | | | | (m, 1 H), 7.55-7.48 (m, 2 H), 7.47-7.34 (m, 1 H), |
| | | | | 7.28 (d, J = 9.2 Hz, 1 H), 7.06 (br d, J = 8.8 Hz, 2 H), |
| | | | | 7.00-6.91 (m, 1 H), 6.87 (dd, J = 2.8, 5.8 Hz, 2 H), |
| | | | | 6.77 (dd, J = 2.8, 9.1 Hz, I H), 6.24-6.10 (m, 2 H), |
| | | | | 4.41-4.29 (m, 4 H), 3.84 (br d, J = 11.6 Hz, 2 H), |
| | | | | 3.73-3.54 (m, 5 H), 3.27-2.92 (m, 11 H), 2.78-2.68 (m, |
| | | | | 7 H), 2.37-2.24 (m, 4 H), 1.85 (br d, J = 11.2 Hz, 2 H), |
| | | | | 1.73-1.53 (m, 6 H) |
| I-1030 | JI | NH | 931.5 | 12.21 (br s, 1 H), 10.83 (br s, 1 H), 8.15-8.09 (m, 1 H), |
| | | | | 7.77-7.63 (m, 1 H), 7.30 (br t, J = 7.2 Hz, 1 H), 7.10- |
| | | | | 7.04 (m, 2 H), 7.02-6.93 (m, 3 H), 6.69-6.63 (m, |
| | | | | 1 H), 6.20-6.12 (m, 1 H), 4.68-4.60 (m, 2 H), 4.40- |
| | | | | 4.30 (m, 2 H), 3.83 (dd, J = 4.8, 11.8 Hz, 1 H), |
| | | | | 3.67-3.49 (m, 4 H), 3.22-2.95 (m, 20 H), 2.70-2.62 (m, |
| | | | | 1 H), 2.36 (br s, 1 H), 2.30-2.16 (m, 2 H), 2.04-1.93 |
| | | | | (m, 3 H) |
| I-1031 | JI | NI | 931.2 | 12.42-12.18 (m, 1 H), 10.89 (s, 1 H), 8.23-8.14 (m, |
| | | | | 1 H), 7.85-7.67 (m, 1 H), 7.40-7.29 (m, 1 H), 7.18- |
| | | | | 6.97 (m, 5 H), 6.72 (br d, J = 2.4 Hz, 1 H), 6.27-6.16 |
| | | | | (m, 1 H), 4.75-4.63 (m, 2 H), 4.50-4.35 (m, 2 H), |
| | | | | 3.88 (dd, J = 4.8, 11.9 Hz, 1 H), 3.73-3.54 (m, 4 H), |
| | | | | 3.27-2.91 (m, 20 H), 2.77-2.68 (m, 1 H), 2.42 (br s, |
| | | | | 1 H), 2.36-2.20 (m, 2 H), 2.11-1.97 (m, 3 H) |
| I-1032^{e} | JI | LN | 811.7 | 12.50-12.10 (m, 1 H), 10.84 (s, 1 H), 8.23-7.95 (m, |
| | | | | 1 H), 7.90-7.48 (m, 1 H), 7.41-7.33 (m, 2 H), |
| | | | | 7.13-6.99 (m, 4 H), 6.70-6.64 (m, 1 H), 6.22-6.13 (m, |
| | | | | 1 H), 4.69-4.59 (m, 2 H), 4.40-4.29 (m, 2 H), |
| | | | | 3.87-3.79 (m, 1 H), 3.69-3.59 (m, 2 H), 3.52 (d, J = 11.4 |
| | | | | Hz, 2 H), 3.23-3.16 (m, 2 H), 3.14-3.04 (m, 6 H), |
| | | | | 2.90-2.82 (m, 2 H), 2.69-2.64 (m, 1 H), 2.39-2.31 |
| | | | | (m, 2 H), 2.29-2.16 (m, 2 H), 2.08-1.98 (m, 2 H), |
| | | | | 1.96 (s, 4 H) |
| I-1033^{d} | NK | AR | 929.5 | 12.29 (br s, 1 H), 10.83 (s, 1 H), 7.72 (dd, J = 2.0, 11.7 |
| | | | | Hz, 1 H), 7.55-7.30 (m, 3 H), 7.18-6.92 (m, 4 H), |
| | | | | 6.68 (br d, J = 2.4 Hz, 1 H), 6.29-6.12 (m, 2 H), |
| | | | | 4.43-4.26 (m, 4 H), 3.81 (dd, J = 4.8, 11.6 Hz, I H), |
| | | | | 3.71-3.50 (m, 3 H), 3.43 (br d, J = 11.2 Hz, 2 H), 3.32-2.93 |
| | | | | (m, 11 H), 2.82-2.56 (m, 6 H), 2.36-2.15 (m, 4 H), |
| | | | | 2.06-1.83 (m, 4 H), 1.74-1.58 (m, 2 H) |
| I-1034 | NM | FV | 789.4 | 8.76-8.72 (m, 1 H), 7.78-7.74 (m, 1 H), 7.74-7.68 |
| | | | | (m, 1 H), 7.61-7.56 (m, 2 H), 7.36 (d, J = 8.3 Hz, 3 H), |
| | | | | 7.04-6.93 (m, 1 H), 6.87-6.83 (m, 1 H), 6.22-6.10 |
| | | | | (m, 2 H), 4.56-4.50 (m, 1 H), 4.38-4.29 (m, 4 H), |
| | | | | 3.81-3.71 (m, 3 H), 3.64 (t, J = 6.0 Hz, 1 H), 3.57 |
| | | | | (t, J = 6.0 Hz, 2 H), 3.11-3.03 (m, 3 H), 3.00-2.95 (m, |
| | | | | 4 H), 2.85-2.78 (m, 1 H), 2.76-2.71 (m, 2 H), 2.67 (s, |
| | | | | 1 H), 2.34-2.24 (m, 4 H), 1.93-1.86 (m, 1 H), 1.84- |
| | | | | 1.78 (m, 2 H), 1.75-1.59 (m, 3 H), 1.52-1.39 (m, 2 H) |
| I-1035 | JI | NO | 793.3 | 12.55-11.81 (m, 1 H), 10.91-10.71 (m, 1 H), |
| | | | | 8.15-8.07 (m, 1 H), 7.73-7.64 (m, 1 H), 7.53 (dt, J = 3.1, |
| | | | | 7.9 Hz, 1 H), 7.35-7.27 (m, 2 H), 7.10-6.98 (m, 4 H), |
| | | | | 6.64-6.58 (m, 1 H), 6.19-6.12 (m, 1 H), 4.67-4.59 |
| | | | | (m, 2 H), 4.40-4.31 (m, 2 H), 3.82 (dd, J = 4.8, 12.0 |
| | | | | Hz, 1 H), 3.67-3.58 (m, 2 H), 3.49 (br d, J = 11.2 Hz, |
| | | | | 2 H), 3.20-3.00 (m, 8 H), 2.86-2.77 (m, 3 H), |
| | | | | 2.71-2.57 (m, 2 H), 2.35-2.17 (m, 3 H), 2.03-1.86 (m, 5 H) |
| I-1037^{c,d} | OW | AR | 930.4 | 12.48-12.06 (m, 1 H), 10.86 (br s, 1 H), 7.89 (s, 1 H), |
| | | | | 7.72 (dd, J = 2.0, 12.0 Hz, 1 H), 7.56-7.29 (m, 4 H), |
| | | | | 7.17-7.05 (m, 1 H), 6.67 (br d, J = 2.4 Hz, 1 H), |
| | | | | 6.24-6.10 (m, 2 H), 4.45-4.23 (m, 4 H), 4.03 (br d, J = 12.0 |
| | | | | Hz, 2 H), 3.86 (dd, J = 4.8, 12.4 Hz, 1 H), 3.69-3.42 |
| | | | | (m, 3 H), 3.26-2.93 (m, 10 H), 2.86 (br t, J = 11.6 Hz, |
| | | | | 2 H), 2.79-2.63 (m, 3 H), 2.56 (br d, J = 3.6 Hz, 1 H), |
| | | | | 2.46 (br s, 1 H), 2.38-2.23 (m, 3 H), 2.22-2.09 (m, |
| | | | | 1 H), 2.06-1.96 (m, 1 H), 1.93-1.81 (m, 3 H), |
| | | | | 1.66-1.51 (m, 2 H) |
| I-1038^{c,d} | OZ | AR | 930.4 | 12.46-12.06 (m, 1 H), 11.08-10.68 (m, 1 H), 7.89 |
| | | | | (s, 1 H), 7.72 (dd, J = 2.0, 11.9 Hz, 1 H), 7.51-7.30 |
| | | | | (m, 4 H), 7.18-7.01 (m, 1 H), 6.73-6.60 (m, 1 H), 6.24- |
| | | | | 6.10 (m, 2 H), 4.43-4.27 (m, 4 H), 4.03 (br d, J = 12.0 |
| | | | | Hz, 2 H), 3.86 (dd, J = 4.8, 12.7 Hz, 1 H), 3.68-3.45 |
| | | | | (m, 3 H), 3.24-2.93 (m, 10 H), 2.86 (brt, J = 11.6 Hz, |
| | | | | 2 H), 2.78-2.62 (m, 3 H), 2.56 (br d, J = 3.6 Hz, 1 H), |
| | | | | 2.47 (br s, 1 H), 2.37-2.23 (m, 3 H), 2.22-2.09 (m, |
| | | | | 1 H), 2.06-1.95 (m, 1 H), 1.95-1.79 (m, 3 H), |
| | | | | 1.66-1.51 (m, 2 H) |
| I-1009 | JE | AH | 824.5 | 12.36-11.90 (m, 1 H), 10.89 (s, 1 H), 7.76-7.67 |
| | | | | (m, 1 H), 7.37 (s, 2 H), 7.33 (s, 1 H), 7.26 (s, 2 H), |
| | | | | 7.13-7.02 (m, 2 H), 6.92-6.79 (m, 1 H), 6.39 (br s, 1 H), |
| | | | | 6.26-6.10 (m, 2 H), 4.40-4.35 (m, 4 H), 4.33 (s, 3 H), |
| | | | | 3.69-3.52 (m, 2 H), 3.42-3.33 (m, 3 H), 3.24-3.01 |
| | | | | (m, 6 H), 3.01-2.96 (m, 2 H), 2.94-2.84 (m, 2 H), |
| | | | | 2.82-2.73 (m, 1 H), 2.69-2.60 (m, 2 H), 2.44-2.21 |
| | | | | (m, 4 H), 2.18 (s, 3 H), 2.02 (br s, 4 H) |
| I-1007 | LD | AR | 824.3 | 12.35-11.87 (m, 1 H), 11.29-10.68 (m, 1 H), |
| | | | | 7.77-7.67 (m, 1 H), 7.46 (d, J = 2.0 Hz, 1 H), 7.32 (s, 1 H), |
| | | | | 7.30-7.23 (m, 3 H), 7.21-7.17 (m, 1 H), 6.92-6.80 |
| | | | | (m, 1 H), 6.72-6.65 (m, 1 H), 6.40 (d, J = 2.8 Hz, 1 H), |
| | | | | 6.24-6.11 (m, 2 H), 5.75-5.65 (m, 1 H), 4.40-4.34 |
| | | | | (m, 3 H), 3.68-3.55 (m, 2 H), 3.53-3.43 (m, 2 H), |
| | | | | 3.28-3.01 (m, 6 H), 3.01-2.90 (m, 3 H), 2.90-2.75 |
| | | | | (m, 4 H), 2.73 (d, J = 2.8 Hz, 2 H), 2.68 (s, 3 H), 2.24 |
| | | | | (d, J = 14.0 Hz, 3 H), 2.18 (s, 3 H), 2.06-1.95 (m, 4 H) |
| I-1036^{c,d} | OS | AR | 929.4 | 12.34-12.24 (m, 1 H), 10.82 (s, 1 H), 7.72 (dd, J = 2.0, |
| | | | | 12.0 Hz, 1 H), 7.45-7.35 (m, 3 H), 7.15-7.06 (m, |
| | | | | 1 H), 7.06-6.95 (m, 3 H), 6.68 (br d, J = 2.4 Hz, 1 H), |
| | | | | 6.23-6.14 (m, 2 H), 4.40-4.31 (m, 4 H), 3.81 (dd, |
| | | | | J = 4.8, 11.6 Hz, 1 H), 3.67-3.51 (m, 3 H), 3.43 |
| | | | | (br d, J = 11.2 Hz, 2 H), 3.29-3.22 (m, 2 H), 3.17 (br d, J = 8.0 |
| | | | | Hz, 2 H), 3.08 (br d, J = 10.4 Hz, 4 H), 2.98 (q, J = 7.6 |
| | | | | Hz, 3 H), 2.80-2.65 (m, 4 H), 2.64-2.59 (m, 1 H), |
| | | | | 2.48 (br s, 1 H), 2.35-2.25 (m, 2 H), 2.23-2.13 |
| | | | | (m, 2 H), 2.01 (td, J = 4.4, 8.8 Hz, 1 H), 1.92-1.83 |
| | | | | (m, 3 H), 1.72-1.62 (m, 2 H) |
| I-1039 | PB | AR | 894.5 | 12.43-12.01 (m, 1 H), 11.00-10.64 (m, 1 H), |
| | | | | 7.92-7.85 (m, 1 H), 7.78-7.66 (m, 1 H), 7.53-7.32 |
| | | | | (m, 3 H), 7.31-7.23 (m, 1 H), 7.11-7.01 (m, 1 H), 6.66 |
| | | | | (s, 1 H), 6.24-6.10 (m, 2 H), 4.40-4.28 (m, 4 H), 4.02 |
| | | | | (d, J = 12.0 Hz, 2 H), 3.86 (d, J = 4.4 Hz, 1 H), 3.65 |
| | | | | (t, J = 5.6 Hz, 1 H), 3.58 (t, J = 5.6 Hz, 1 H), 3.24-3.07 |
| | | | | (m, 6 H), 3.04 (d, J = 9.6 Hz, 3 H), 3.01-2.92 (m, 3 H), |
| | | | | 2.89-2.80 (m, 3 H), 2.75-2.64 (m, 2 H), 2.56 (d, J = |
| | | | | 3.6 Hz, 1 H), 2.37-2.31 (m, 3 H), 2.27-2.23 (m, 1 H), |
| | | | | 2.03-1.97 (m, 1 H), 1.89-1.79 (m, 4 H), 1.76-1.72 |
| | | | | (m, 1 H), 1.63-1.49 (m, 2 H) |
| I-1040 | JI | PF | 810.3 | 12.41-11.89 (m, 1 H), 10.92-10.75 (m, 1 H), |
| | | | | 7.74-7.64 (m, 3 H), 7.55-7.47 (m, 3 H), 7.45-7.35 |
| | | | | (m, 1 H), 7.10 (d, J = 8.8 Hz, 2 H), 7.06-7.00 (m, 1 H), |
| | | | | 6.89 (s, 1 H), 6.19 (d, J = 13.6 Hz, 2 H), 4.41-4.30 (m, |
| | | | | 4 H), 3.91 (s, 1 H), 3.74-3.62 (m, 2 H), 3.61-3.54 (m, |
| | | | | 2 H), 3.24-3.04 (m, 6 H), 3.01-2.94 (m, 3 H), |
| | | | | 2.69-2.63 (m, 1 H), 2.40-2.30 (m, 3 H), 2.29-2.17 |
| | | | | (m, 2 H), 2.05-1.97 (m, 2 H), 1.35-1.15 (m, 1 H) |
| I-1041^{c} | PH | AK | 809.4 | 12.28-12.12 (m, 1 H), 10.97-10.86 (m, 1 H), |
| | | | | 8.51-8.44 (m, 1 H), 8.16-8.08 (m, 1 H), 7.73-7.66 |
| | | | | (m, 1 H), 7.65-7.60 (m, 2 H), 7.44 (d, J = 8.0 Hz, 2 H), |
| | | | | 7.14-7.01 (m, 3 H), 6.91-6.85 (m, 1 H), 6.22-6.14 |
| | | | | (m, 1 H), 4.66-4.59 (m, 2 H), 4.43-4.33 (m, 2 H), |
| | | | | 4.24-4.14 (m, 1 H), 3.70-3.59 (m, 2 H), 3.55-3.49 |
| | | | | (m, 2 H), 3.26-3.01 (m, 9 H), 2.88 (br t, J = 10.8 Hz, |
| | | | | 2 H), 2.82-2.73 (m, 2 H), 2.56 (br d, J = 3.2 Hz, 1 H), |
| | | | | 2.35-2.32 (m, 1 H), 2.30-2.26 (m, 1 H), 2.00-1.85 |
| | | | | (m, 5 H) |
| I-1042 | PL | AR | 894.5 | 12.20 (d, J = 8.5 Hz, 1 H), 10.82 (s, 1 H), 7.84-7.59 |
| | | | | (m, 1 H), 7.53-7.23 (m, 2 H), 7.12 (t, J = 8.7 Hz, 1 H), |
| | | | | 7.04-6.94 (m, 2 H), 6.78-6.69 (m, 2 H), 6.64 (s, 1 H), |
| | | | | 6.23-6.10 (m, 2 H), 4.40-4.34 (m, 3 H), 4.28 (s, 1 H), |
| | | | | 3.95-3.85 (m, 1 H), 3.64 (t, J = 5.6 Hz, 1 H), 3.60-3.52 |
| | | | | (m, 3 H), 3.16 (s, 6 H), 3.10-3.04 (m, 2 H), 3.02-2.91 |
| | | | | (m, 3 H), 2.83-2.74 (m, 2 H), 2.74-2.57 (m, 6 H), |
| | | | | 2.47-2.39 (m, 2 H), 2.35-2.25 (m, 2 H), 2.20-2.08 |
| | | | | (m, 1 H), 1.99-1.87 (m, 3 H), 1.68-1.56 (m, 2 H) |
| I-1043 | MF | AK | 793.4 | 12.19 (br d, J = 6.4 Hz, 1 H), 10.87 (s, 1 H), 8.16-8.05 |
| | | | | (m, 1 H), 7.73-7.56 (m, 3 H), 7.44 (d, J = 8.0 Hz, 2 H), |
| | | | | 7.15 (dd, J = 6.8, 13.2 Hz, 1 H), 7.09-7.00 (m, 1 H), |
| | | | | 6.96-6.85 (m, 2 H), 6.29-6.06 (m, 1 H), 4.71-4.52 |
| | | | | (m, 2 H), 4.44-4.23 (m, 2 H), 4.08-3.88 (m, 1 H), |
| | | | | 3.74-3.47 (m, 4 H), 3.21-3.15 (m, 2 H), 3.14-3.07 |
| | | | | (m, 4 H), 2.88-2.72 (m, 4 H), 2.18 (br s, 5 H), |
| | | | | 2.02-1.83 (m, 6 H) |
| I-1044^{c} | PN | AR | 912.3 | 12.30-12.04 (m, 1 H), 10.87 (s, 1 H), 7.84-7.65 (m, |
| | | | | 1 H), 7.38 (s, 1 H), 7.33-7.24 (m, 1 H), 7.13 (dd, J = |
| | | | | 6.8, 13.2 Hz, 1 H), 7.05-6.92 (m, 2 H), 6.87 (dd, J = |
| | | | | 7.6, 12.0 Hz, 1 H), 6.71-6.62 (m, 1 H), 6.27-6.11 (m, |
| | | | | 2 H), 4.41-4.29 (m, 4 H), 4.02-3.92 (m, 1 H), |
| | | | | 3.67-3.57 (m, 2 H), 3.46 (br d, J = 11.2 Hz, 2 H), 3.15 |
| | | | | (br s, 7 H), 3.08-2.90 (m, 5 H), 2.84-2.64 (m, 8 H), |
| | | | | 2.46-2.28 (m, 3 H), 2.24-2.12 (m, 1 H), 2.04-1.88 (m, |
| | | | | 3 H), 1.71-1.56 (m, 2 H) |
| I-1045^{c,d} | PP | OP | 811.4 | 10.81 (s, 1 H), 8.12-8.09 (m, 1 H), 7.71-7.67 (m, |
| | | | | 1 H), 7.63-7.60 (m, 1 H), 7.44 (s, 4 H), 7.07-7.06 (m, |
| | | | | 1 H), 7.05-7.02 (m, 1 H), 7.00-6.97 (m, 2 H), 6.78 (s, |
| | | | | 1 H), 6.10-6.02 (m, 1 H), 4.63 (t, J = 6.8 Hz, 2 H), 4.23 |
| | | | | (s, 2 H), 3.82 (dd, J = 4.8, 12.0 Hz, 1 H), 3.67-3.57 |
| | | | | (m, 4 H), 3.50 (d, J = 12.0 Hz, 2 H), 3.11 (d, J = 8.4 Hz, |
| | | | | 2 H), 3.00 (s, 6 H), 2.87-2.79 (m, 3 H), 2.66-2.60 (m, |
| | | | | 1 H), 2.29-2.17 (m, 3 H), 2.07-1.97 (m, 2 H), |
| | | | | 1.95-1.87 (m, 4 H), 0.50-0.42 (m, 1 H), 0.05-0.00 |
| | | | | (m, 2 H), −0.30--0.34 (m, 2 H) |
| I-1046 | MF | PQ | 807.3 | 12.19 (br d, J = 6.4 Hz, 1 H), 10.86 (s, 1 H), 8.14-8.08 |
| | | | | (m, 1 H), 7.72-7.65 (m, 1 H), 7.62 (br d, J = 8.0 Hz, |
| | | | | 2 H), 7.43 (d, J = 8.4 Hz, 2 H), 7.15 (dd, J = 7.2, 13.6 |
| | | | | Hz, 1 H), 7.09-7.00 (m, 1 H), 6.95-6.89 (m, 1 H), |
| | | | | 6.84 (br s, 1 H), 6.20-6.14 (m, 1 H), 4.65-4.60 (m, |
| | | | | 2 H), 4.40-4.32 (m, 2 H), 3.98 (dd, J = 4.8, 12.8 Hz, |
| | | | | 1 H), 3.68-3.59 (m, 2 H), 3.53 (br d, J = 9.6 Hz, 4 H), |
| | | | | 3.15-3.06 (m, 4 H), 2.83 (br t, J = 10.8 Hz, 2 H), |
| | | | | 2.78-2.69 (m, 2 H), 2.41-2.31 (m, 2 H), 2.30-2.25 (m, |
| | | | | 1 H), 2.24-2.16 (m, 1 H), 2.03-1.81 (m, 6 H), 1.16 (t, |
| | | | | J = 7.2 Hz, 3 H) |
| I-1047 | NP | PQ | 826.5 | 12.31-12.16 (m, 1 H), 10.88-10.79 (m, 1 H), |
| | | | | 8.52-8.50 (m, 1 H), 8.16-8.06 (m, 1 H), 7.73-7.59 (m, |
| | | | | 1 H), 7.38-7.29 (m, 1 H), 7.12-7.00 (m, 5 H), |
| | | | | 6.70-6.56 (m, 1 H), 6.21-6.11 (m, 1 H), 4.69-4.60 |
| | | | | (m, 2 H), 4.40-4.31 (m, 2 H), 3.88-3.80 (m, 1 H), |
| | | | | 3.71-3.59 (m, 2 H), 3.56-3.48 (m, 2 H), 3.30-3.27 (m, |
| | | | | 6 H), 3.23 (br d, J = 4.0 Hz, 4 H), 3.14-3.06 (m, 4 H), |
| | | | | 2.69-2.63 (m, 1 H), 2.36-2.32 (m, 1 H), 2.31-2.26 |
| | | | | (m, 1 H), 2.24-2.16 (m, 1 H), 2.08-2.00 (m, 1 H), |
| | | | | 1.18-1.12 (m, 3 H) |
| I-1416 | SL | AR | 853.6 | 12.22-12.06 (m, 1 H), 11.13-10.98 (m, 1 H), |
| | | | | 8.54-8.44 (m, 1 H), 7.76-7.62 (m, 1 H), 7.44-7.35 (m, 1 H), |
| | | | | 7.30-7.18 (m, 4 H), 7.15-7.08 (m, 1 H), 6.91-6.78 |
| | | | | (m, 2 H), 6.44-6.32 (m, 1 H), 6.24-6.06 (m, 2 H), |
| | | | | 5.46-5.05 (m, 1 H), 4.40-4.28 (m, 4 H), 3.65-3.55 (m, |
| | | | | 2 H), 3.21-3.02 (m, 6 H), 2.99-2.93 (m, 6 H), 2.91 (br |
| | | | | s, 2 H), 2.68-2.60 (m, 2 H), 2.34-2.24 (m, 2 H), |
| | | | | 2.20-2.14 (m, 3 H), 2.00-1.88 (m, 4 H), 1.88 (br s, 1 H), |
| | | | | 1.46 (br d, J = 2.8 Hz, 6 H) |
| I-1417 | SL | AK | 854.8 | 12.23-12.06 (m, 1 H), 11.20-10.89 (m, 1 H), 8.54-8.45 |
| | | | | (m, 1 H), 8.14-8.06 (m, 1 H), 7.73-7.64 (m, 1 H), |
| | | | | 7.30-7.21 (m, 4 H), 7.14-7.10 (m, 1 H), 6.92-6.76 |
| | | | | (m, 2 H), 6.41-6.35 (m, 1 H), 6.19-6.11 (m, 1 H), |
| | | | | 5.32-5.10 (m, 1 H), 4.65-4.58 (m, 2 H), 4.39-4.30 (m, |
| | | | | 2 H), 3.70-3.55 (m, 2 H), 3.15-3.04 (m, 6 H), 2.85 (br |
| | | | | s, 1 H), 2.99-2.84 (m, 6 H), 2.77-2.69 (m, 1 H), |
| | | | | 2.66-2.57 (m, 2 H), 2.38-2.23 (m, 3 H), 2.17 (s, 3 H), |
| | | | | 2.02-1.78 (m, 6 H), 1.48-1.43 (m, 6 H) |
| I-1418 | SL | PQ | 868.4 | 12.25-12.05 (m, I H), 11.33-10.82 (m, 1 H), |
| | | | | 8.15-8.06 (m, 1 H), 7.72-7.63 (m, 1 H), 7.30-7.20 (m, 4 H), |
| | | | | 7.16-7.09 (m, 1 H), 6.92-6.80 (m, 2 H), 6.40-6.30 |
| | | | | (m, 1 H), 6.19-6.10 (m, 1 H), 5.28-5.19 (m, 1 H), 4.64-4.59 |
| | | | | (m, 2 H), 4.38 (br s, 1 H), 4.32 (br s, 1 H), 4.40-4.29 |
| | | | | (m, 1 H), 3.64-3.61 (m, 2 H), 3.18-3.03 (m, 6 H), |
| | | | | 3.00-2.89 (m, 6 H), 2.76-2.69 (m, 2 H), 2.36-2.26 |
| | | | | (m, 2 H), 2.21-2.13 (m, 3 H), 2.01-1.89 (m, 4 H), 1.87-1.81 |
| | | | | (m, 1 H), 1.52-1.42 (m, 6 H), 1.17-1.05 (m, 3 H) |
| I-1419^{c} | PP | SN | 842.2 | 10.80-10.73 (m, 1 H), 8.10 (br s, 1 H), 7.69 (s, 1 H), |
| | | | | 7.59 (br d, J = 6.4 Hz, 1 H), 7.31-7.24 (m, 1 H), |
| | | | | 7.23-7.17 (m, 1 H), 7.04-6.99 (m, 1 H), 6.98-6.93 (m, 2 H), |
| | | | | 6.88 (br d, J = 6.4 Hz, 1 H), 6.79-6.62 (m, 1 H), |
| | | | | 6.56-6.49 (m, 1 H), 6.11-5.99 (m, 1 H), 4.91-4.83 (m, 1 H), |
| | | | | 4.63 (br t, J = 6.4 Hz, 2 H), 4.27-4.20 (m, 2 H), |
| | | | | 4.09-4.02 (m, 1 H), 3.94-3.84 (m, 2 H), 3.82-3.67 (m, 5 H), |
| | | | | 3.67-3.49 (m, 3 H), 3.14-3.05 (m, 4 H), 2.99 (br s, |
| | | | | 6 H), 2.35-2.27 (m, 3 H), 2.21-2.14 (m, 1 H), |
| | | | | 2.04-1.97 (m, 1 H), 0.53-0.43 (m, 1 H), 0.04 |
| | | | | (br d, J = 2.4 Hz, 2 H), −0.23--0.31 (m, 2 H) |
| I-1420^{c,d} | PP | SP | 825.5 | 10.78 (br s, 1 H), 8.09 (d, J = 2.8 Hz, 1 H), 7.69-7.65 |
| | | | | (m, 1 H), 7.61-7.58 (m, 1 H), 7.41 (br d, J = 7.6 Hz, |
| | | | | 2 H), 7.31 (br d, J = 7.6 Hz, 2 H), 7.00-6.91 (m, 4 H), |
| | | | | 6.76-6.60 (m, 1 H), 6.07-5.98 (m, 1 H), 4.61 |
| | | | | (t, J = 6.8 Hz, 2 H), 4.21 (br s, 2 H), 3.77 (dd, J = 4.8, 11.6 |
| | | | | Hz, 1 H), 3.69-3.52 (m, 5 H), 3.11-3.06 (m, 2 H), |
| | | | | 2.99 (s, 6 H), 2.66 (br d, J = 6.0 Hz, 3 H), 2.63-2.55 |
| | | | | (m, 4 H), 2.32-2.23 (m, 2 H), 2.20-2.12 (m, 1 H), 1.97 |
| | | | | (td, J = 4.4, 8.8 Hz, 1 H), 1.68 (br d, J = 10.4 Hz, 3 H), |
| | | | | 1.43-1.34 (m, 2 H), 0.47-0.39 (m, 1 H), 0.00 |
| | | | | (br t, J = 6.4 Hz, 2 H), −0.33 (br dd, J = 4.4, 8.0 Hz, 2 H) |
| I-1421^{c,d} | PP | SR | 812.7 | 10.87 (s, 1 H), 8.10 (s, 1 H), 7.91 (s, 1 H), 7.69 (s, 1 H), |
| | | | | 7.64-7.58 (m, 1 H), 7.53-7.31 (m, 6 H), 6.97 (s, 1 H), |
| | | | | 6.80-6.60 (m, 1 H), 6.02 (s, 1 H), 4.63 (t, J = 6.4 Hz, |
| | | | | 2 H), 4.23 (s, 2 H), 4.11 (d, J = 12.4 Hz, 2 H), 3.87 (dd, |
| | | | | J = 4.8, 12.8 Hz, 1 H), 3.67-3.55 (m, 4 H), 3.14-3.07 |
| | | | | (m, 2 H), 3.03 (br s, 1 H), 3.00 (s, 6 H), 2.97 (br s, 1 H), |
| | | | | 2.88 (br s, 1 H), 2.68 (dd, J = 5.2, 12.4 Hz, 1 H), |
| | | | | 2.34-2.22 (m, 3 H), 1.94 (d, J = 12.0 Hz, 3 H), 1.81 |
| | | | | (d, J = 10.4 Hz, 2 H), 0.55-0.34 (m, 1 H), 0.07--0.06 |
| | | | | (m, 2 H), −0.26--0.36 (m, 2 H) |
| I-1426 | SX | AK | 789.4 | 12.19 (br d, J = 5.6 Hz, 1 H), 10.92 (s, 1 H), 8.14-8.09 |
| | | | | (m, 1 H), 7.72-7.67 (m, 1 H), 7.63 (br d, J = 8.0 Hz, |
| | | | | 2 H), 7.44 (d, J = 8.1 Hz, 2 H), 7.15-6.98 (m, 4 H), |
| | | | | 6.88 (br s, 1 H), 6.17 (br s, 1 H), 4.68-4.59 (m, 2 H), |
| | | | | 4.42-4.32 (m, 2 H), 3.69-3.64 (m, 1 H), 3.61 |
| | | | | (br t, J = 5.6 Hz, 1 H), 3.51 (br d, J = 10.0 Hz, 2 H), |
| | | | | 3.25-3.15 (m, 3 H), 3.14-3.04 (m, 5 H), 2.80 (br d, J = 11.2 |
| | | | | Hz, 2 H), 2.77-2.70 (m, 1 H), 2.45-2.26 (m, 4 H), |
| | | | | 2.15-2.05 (m, 2 H), 1.97-1.85 (m, 4 H), 1.43 (s, 3 H) |
| I-1427^{c} | TA | PP | 844.6 | 10.79 (s, 1 H), 8.11 (d, J = 1.2 Hz, 1 H), 7.69 (s, 1 H), |
| | | | | 7.61-7.54 (m, 1 H), 7.24 (dd, J = 2.0, 14.4 Hz, 1 H), |
| | | | | 7.18-7.13 (m, 1 H), 7.12-7.05 (m, 1 H), 6.99 (s, 1 H), |
| | | | | 6.98-6.94 (m, 2 H), 6.91-6.87 (m, 1 H), 6.79-6.59 |
| | | | | (m, 1 H), 6.10-5.99 (m, 1 H), 4.63 (t, J = 6.8 Hz, 2 H), |
| | | | | 4.22 (br d, J = 4.4 Hz, 2 H), 3.76 (dd, J = 5.2, 11.6 Hz, |
| | | | | 1 H), 3.73-3.66 (m, 2 H), 3.63 (s, 3 H), 3.60-3.56 (m, |
| | | | | 1 H), 3.55-3.48 (m, 4 H), 3.40 (s, 2 H), 3.11 (q, J = 6.8 |
| | | | | Hz, 2 H), 2.99 (s, 6 H), 2.68-2.59 (m, 1 H), 2.48-2.42 |
| | | | | (m, 1 H), 2.34-2.25 (m, 2 H), 2.17 (dt, J = 8.4, 12.0 |
| | | | | Hz, 1 H), 2.08-1.97 (m, 3 H), 0.55-0.39 (m, 1 H), |
| | | | | 0.09--0.06 (m, 2 H), −0.25--0.35 (m, 2 H) |
| I-1428 | SL | TB | 867.5 | 12.14 (d, J = 5.6 Hz, I H), 11.07 (s, 1 H), 7.68-7.59 |
| | | | | (m, 1 H), 7.32-7.22 (m, 4 H), 7.12 (d, J = 8.4 Hz, 1 H), |
| | | | | 6.89-6.78 (m, 2 H), 6.39 (s, 1 H), 6.23-6.07 (m, 2 H), |
| | | | | 5.32-5.15 (m, 1 H), 4.40-4.17 (m, 3 H), 4.14-4.02 |
| | | | | (m, 1 H), 3.69-3.43 (m, 3 H), 3.18-3.02 (m, 5 H), |
| | | | | 3.00-2.81 (m, 6 H), 2.79-2.55 (m, 4 H), 2.34-2.20 (m, |
| | | | | 2 H), 2.17 (d, J = 5.6 Hz, 3 H), 1.99-1.84 (m, 5 H), |
| | | | | 1.46 (d, J = 2.4 Hz, 6 H), 1.04-0.96 (m, 3 H) |
| I-1429 | SL | FS | 867.5 | 12.15 (d, J = 6.0 Hz, 1 H), 11.08 (s, 1 H), 7.69-7.59 |
| | | | | (m, 1 H), 7.33-7.23 (m, 4 H), 7.13 (d, J = 8.0 Hz, 1 H), |
| | | | | 6.90-6.76 (m, 2 H), 6.40 (s, 1 H), 6.23-6.07 (m, 2 H), |
| | | | | 5.24 (td, J = 2.0, 4.6 Hz, 1 H), 4.41-4.18 (m, 3 H), |
| | | | | 4.14-4.04 (m, 1 H), 3.69-3.44 (m, 3 H), 3.23-3.03 |
| | | | | (m, 5 H), 3.01-2.83 (m, 6 H), 2.80-2.54 (m, 4 H), |
| | | | | 2.35-2.20 (m, 2 H), 2.18 (d, J = 5.6 Hz, 3 H), 2.00-1.82 |
| | | | | (m, 5 H), 1.47 (d, J = 2.8 Hz, 6 H), 1.06-0.97 (m, 3 H) |
| I-1430^{c,d} | SR | TC | 811.3 | 10.86 (s, 1 H), 7.91 (s, 1 H), 7.71 (s, 1 H), 7.61 (d, J = |
| | | | | 5.6 Hz, 2 H), 7.49 (s, 1 H), 7.46-7.39 (m, 5 H), 6.97 (s, |
| | | | | 1 H), 6.81-6.59 (m, 1 H), 6.19 (s, 1 H), 6.10-5.97 (m, |
| | | | | 1 H), 4.37 (t, J = 6.8 Hz, 2 H), 4.26-4.17 (m, 2 H), 4.11 |
| | | | | (d, J = 12.4 Hz, 2 H), 3.87 (dd, J = 4.0, 12.4 Hz, 1 H), |
| | | | | 3.68-3.54 (m, 4 H), 3.03 (s, 1 H), 3.00 (s, 6 H), |
| | | | | 2.98-2.94 (m, 2 H), 2.88 (t, J = 12.4 Hz, 1 H), 2.77-2.60 |
| | | | | (m, 2 H), 2.34-2.23 (m, 3 H), 2.04-1.89 (m, 3 H), |
| | | | | 1.86-1.74 (m, 2 H), 0.52-0.35 (m, 1 H), 0.02 (t, J = 6.0 |
| | | | | Hz, 2 H), −0.33 (d, J = 4.4 Hz, 2 H) |
| I-1431^{c} | PP | TD | 847.4 | 10.88 (s, 1 H), 8.11 (d, J = 2.8 Hz, 1 H), 7.69 (d, J = |
| | | | | 2.0 Hz, 1 H), 7.65-7.58 (m, 1 H), 7.48 (s, 4 H), 7.40 (d, |
| | | | | J = 7.6 Hz, 1 H), 7.13-7.02 (m, 2 H), 6.99 (d, J = 1.2 |
| | | | | Hz. 1 H), 6.83-6.58 (m, 1 H), 6.21-5.92 (m, 1 H), |
| | | | | 4.63 (t, J = 6.8 Hz, 2 H), 4.38-4.34 (m, 1 H), 4.33 (s, |
| | | | | 3 H), 4.24 (s, 2 H), 3.76-3.50 (m, 4 H), 3.37 (d, J = 4.4 |
| | | | | Hz, 2 H), 3.15-3.06 (m, 2 H), 3.01 (s, 6 H), 2.91 |
| | | | | (dd, J = 5.8, 9.2 Hz, 3 H), 2.68-2.62 (m, 2 H), 2.32 (d, J = |
| | | | | 2.0 Hz, 3 H), 2.23-2.13 (m, 1 H), 2.07-1.97 (m, 4 H), |
| | | | | 0.61-0.36 (m, 1 H), 0.03 (t, J = 6.4 Hz, 2 H), −0.23--0.47 |
| | | | | (m, 2 H) |
| I-1432^{c} | TE | TD | 766.4 | 10.89 (s, 1 H), 7.62 (s, 1 H), 7.48 (s, 4 H), 7.40 (d, J = |
| | | | | 7.6 Hz, 1 H), 7.13-7.02 (m, 2 H), 6.99 (s, 1 H), |
| | | | | 6.81-6.58 (m, 1 H), 6.19-5.93 (m, 1 H), 4.39-4.34 (m, 1 H), |
| | | | | 4.32 (s, 3 H), 4.22 (s, 2 H), 3.74-3.64 (m, 2 H), 3.59 (q, |
| | | | | J = 5.6 Hz, 2 H), 3.38 (d, J = 10.0 Hz, 2 H), 3.01 (s, |
| | | | | 6 H), 2.96-2.78 (m, 3 H), 2.72-2.60 (m, 2 H), 2.35 |
| | | | | (dd, J = 4.8, 9.2 Hz, 2 H), 2.26 (s, 1 H), 2.18 (dd, J = |
| | | | | 5.6, 13.6 Hz, 1 H), 2.12-2.06 (m, 3 H), 2.03 (s, 4 H), |
| | | | | 0.48 (dd, J = 4.8, 6.8 Hz, 1 H), 0.12--0.07 (m, 2 H), −0.30 |
| | | | | (gd, J = 4.4, 9.2 Hz, 2 H) |
| I-1433^{c} | TI | OP | 829.4 | 10.83 (s, 1 H), 8.11 (d, J = 3.2 Hz, 1 H), 7.69 (d, J = |
| | | | | 2.4 Hz, 1 H), 7.43 (s, 4 H), 7.15-7.03 (m, 2 H), 7.00 (d, |
| | | | | J = 8.4 Hz, 1 H), 6.92-6.64 (m, 2 H), 6.10 (d, J = 18.0 |
| | | | | Hz, 1 H), 4.63 (t, J = 6.8 Hz, 2 H), 4.24 (s, 2 H), 3.83 |
| | | | | (dd, J = 4.8, 11.6 Hz, 1 H), 3.67 (dd, J = 6.4, 15.6 Hz, |
| | | | | 2 H), 3.62-3.56 (m, 2 H), 3.51 (d, J = 11.6 Hz, 2 H), |
| | | | | 3.16-3.07 (m, 2 H), 3.01 (s, 3 H), 2.93 (s, 3 H), 2.90- |
| | | | | 2.75 (m, 3 H), 2.74-2.56 (m, 2 H), 2.34-2.18 (m, 3 H), |
| | | | | 2.05-1.99 (m, 1 H), 1.95 (s, 3 H), 1.90 (d, J = 4.0 Hz, |
| | | | | 1 H), 0.58-0.40 (m, 1 H), 0.05 (t, J = 7.6 Hz, 2 H), −0.23--0.37 |
| | | | | (m, 2 H) |
| I-1434^{c} | PP | TK | 829.7 | 10.82 (s, 1 H), 8.11 (s, 1 H), 7.69 (s, 1 H), 7.67-7.61 |
| | | | | (m, 1 H), 7.47 (t, J = 7.6 Hz, 1 H), 7.40-7.27 (m, 2 H), |
| | | | | 7.15 (t, J = 7.2 Hz, 1 H), 7.00 (s, 1 H), 6.97-6.81 (m, |
| | | | | 2 H), 6.80-6.63 (m, 1 H), 6.14-6.00 (m, 1 H), 4.63 (t, |
| | | | | J = 6.8 Hz, 2 H), 4.23 (s, 2 H), 3.70-3.53 (m, 5 H), |
| | | | | 3.19-3.07 (m, 4 H), 3.00 (s, 8 H), 2.78-2.65 (m, 3 H), |
| | | | | 2.35-2.25 (m, 2 H), 2.22-2.11 (m, 1 H), 1.92 (s, 5 H), |
| | | | | 0.55-0.42 (m, 1 H), 0.13--0.02 (m, 2 H), −0.28 |
| | | | | (dd, J = 5.2, 8.0 Hz, 2 H) |
| I-1435^{c} | PP | TM | 842.4 | 10.80-10.73 (m, 1 H), 8.12-8.08 (m, 1 H), 7.69 (s, |
| | | | | 1 H), 7.61-7.57 (m, 1 H), 7.27 (br d, J = 14.4 Hz, I H), |
| | | | | 7.20 (dd, J = 1.8, 8.3 Hz, 1 H), 7.05-6.99 (m, 1 H), |
| | | | | 6.98 (s, 2 H), 6.88 (dd, J = 1.7, 8.3 Hz, 1 H), 6.78-6.62 |
| | | | | (m, 1 H), 6.56-6.50 (m, 1 H), 6.10-6.01 (m, 1 H), 4.87 |
| | | | | (td, J = 3.5, 6.7 Hz, 1 H), 4.63 (t, J = 6.8 Hz, 2 H), 4.23 |
| | | | | (br d, J = 5.6 Hz, 2 H), 4.05 (br t, J = 7.3 Hz, 1 H), |
| | | | | 3.93-3.83 (m, 2 H), 3.80-3.70 (m, 4 H), 3.63-3.56 (m, |
| | | | | 2 H), 3.14-3.04 (m, 4 H), 2.99 (s, 7 H), 2.67-2.61 (m, |
| | | | | 1 H), 2.33-2.26 (m, 2 H), 2.20-2.13 (m, 1 H), 2.02-1.97 |
| | | | | (m, 1 H), 1.31-1.15 (m, 1 H), 0.52-0.43 (m, 1 H), |
| | | | | 0.04 (br t, J = 6.4 Hz, 2 H), −0.24--0.29 (m, 2 H) |
| I-1436^{f} | TO | AR | 807.3 | 12.27-12.15 (m, 1 H), 10.84 (s, 1 H), 7.85-7.66 |
| | | | | (m, 2 H), 7.48-7.28 (m, 2 H), 7.12-7.03 (m, 2 H), |
| | | | | 7.02-6.95 (m, 2 H), 6.26-6.10 (m, 2 H), 4.47-4.25 (m, 4 H), |
| | | | | 3.83 (dd, J = 4.8, 11.6 Hz, 1 H), 3.69-3.56 (m, 2 H), |
| | | | | 3.51 (d, J = 11.6 Hz, 2 H), 3.28-3.02 (m, 6 H), |
| | | | | 3.01-2.83 (m, 5 H), 2.73-2.64 (m, 1 H), 2.62 (s, 3 H), 2.53 |
| | | | | (s, 1 H), 2.36-2.15 (m, 3 H), 2.07-1.98 (m, 1 H), 1.91 |
| | | | | (d, J = 2.6 Hz, 4 H) |
| I-1437 | TQ | OP | 775.5 | 10.83 (s, 1 H), 8.15-8.11 (m, 2 H), 7.91 (d, J = 8.4 Hz, |
| | | | | 1 H), 7.86 (d, J = 8.4 Hz, 1 H), 7.74-7.69 (m, 1 H), |
| | | | | 7.64-7.59 (m, 1 H), 7.46 (d, J = 8.4 Hz, 1 H), 7.39 (d, |
| | | | | J = 8.4 Hz, 1 H), 7.13-7.03 (m, 2 H), 7.02-6.92 (m, |
| | | | | 2 H), 4.67-4.59 (m, 2 H), 4.58-4.48 (m, 2 H), 3.83 |
| | | | | (dd, J = 4.8, 11.6 Hz, I H), 3.67 (t, J = 5.6 Hz, 1 H), |
| | | | | 3.62 (t, J = 5.6 Hz, 1 H), 3.50 (d, J = 10.8 Hz, 2 H), |
| | | | | 3.12 (q, J = 7.2 Hz, 2 H), 3.02 (s, 6 H), 2.87 (s, 3 H), |
| | | | | 2.67-2.61 (m, 1 H), 2.47-2.35 (m, 2 H), 2.26-2.21 |
| | | | | (m, 1 H), 2.09-1.81 (m, 6 H) |
| I-1440^{f} | TS | AR | 807.3 | 12.23-12.15 (m, 1 H), 10.83 (s, 1 H), 8.51 (s, 1 H), |
| | | | | 7.78-7.67 (m, 1 H), 7.46-7.36 (m, 1 H), 7.34-7.26 |
| | | | | (m, 1 H), 7.12-7.03 (m, 2 H), 7.02-6.97 (m, 1 H), |
| | | | | 6.94-6.86 (m, 1 H), 6.22-6.16 (m, 1 H), 6.13 (s, 1 H), |
| | | | | 4.42-4.28 (m, 4 H), 3.82 (dd, J = 4.8, 11.6 Hz, 1 H), |
| | | | | 3.68-3.56 (m, 2 H), 3.51 (d, J = 10.4 Hz, 2 H), 3.25-2.93 |
| | | | | (m, 10 H), 2.92-2.84 (m, 2 H), 2.71-2.62 (m, 1 H), |
| | | | | 2.38 (s, 3 H), 2.35-2.14 (m, 3 H), 2.06-1.92 (m, 5 H) |
| I-1441^{c} | TU | OP | 829.6 | 10.77 (s, 1 H), 8.05 (s, 1 H), 7.63 (s, 1 H), 7.54 (d, J = |
| | | | | 1.6 Hz, 1 H), 7.44-7.36 (m, 4 H), 7.05-6.90 (m, 4 H), |
| | | | | 6.15-6.02 (m, 1 H), 4.57 (t, J = 6.8 Hz, 2 H), |
| | | | | 4.25-4.15 (m, 2 H), 3.76 (dd, J = 4.8, 11.6 Hz, 1 H), |
| | | | | 3.61-3.42 (m, 6 H), 3.08-3.00 (m, 2 H), 2.95 (s, 6 H), |
| | | | | 2.83-2.70 (m, 3 H), 2.66-2.57 (m, 1 H), 2.35-2.21 (m, 3 H), |
| | | | | 2.20-2.10 (m, 1 H), 2.00-1.81 (m, 5 H), 0.46-0.34 |
| | | | | (m, 1 H), 0.04--0.04 (m, 2 H), −0.33--0.41 (m, 2 H) |
| I-1443 | TQ | TD | 811.5 | 10.89 (s, 1 H), 8.18-8.08 (m, 2 H), 7.94-7.85 (m, |
| | | | | 2 H), 7.70 (s, 1 H), 7.64-7.57 (m, 1 H), 7.50 (d, J = 8.0 |
| | | | | Hz, 1 H), 7.46-7.37 (m, 2 H), 7.13-7.02 (m, 2 H), |
| | | | | 7.00-6.92 (m, 1 H), 4.68-4.59 (m, 2 H), 4.57-4.48 |
| | | | | (m, 2 H), 4.38-4.34 (m, 1 H), 4.33 (s, 3 H), 3.70-3.65 |
| | | | | (m, 1 H), 3.42-3.35 (m, 2 H), 3.16-3.09 (m, 2 H), 3.01 |
| | | | | (s, 6 H), 2.98-2.71 (m, 4 H), 2.68-2.60 (m, 2 H), 2.45 |
| | | | | (s, 1 H), 2.41-2.28 (m, 2 H), 2.23-2.13 (m, 1 H), |
| | | | | 2.08-1.96 (m, 4 H) |
| I-1444^{c} | PP | NO | 829.2 | 10.83 (s, 1 H), 8.11 (s, 1 H), 7.82-7.61 (m, 2 H), |
| | | | | 7.49-7.44 (m, 1 H), 7.38-7.27 (m, 2 H), 7.11-6.96 (m, 4 H), |
| | | | | 6.83-6.57 (m, 1 H), 6.12-5.95 (m, 1 H), 4.63 (t, J = |
| | | | | 6.8 Hz, 2 H), 4.33-4.10 (m, 2 H), 3.82 (dd, J = 4.8, |
| | | | | 11.6 Hz, 1 H), 3.71-3.55 (m, 4 H), 3.49 (d, J = 11.6 |
| | | | | Hz, 2 H), 3.10 (d, J = 6.8 Hz, 2 H), 3.00 (s, 6 H), 2.82 (t, |
| | | | | J = 11.2 Hz, 3 H), 2.65 (d, J = 12.0 Hz, 2 H), 2.34-2.17 |
| | | | | (m, 3 H), 2.06-1.86 (m, 5 H), 0.64-0.46 (m, 1 H), |
| | | | | 0.16-0.01 (m, 2 H), −0.28 (td, J = 4.4, 9.2 Hz, 2 H) |
| I-1445 | PP | TW | 825.2 | 10.83 (s, 1 H), 8.11-8.10 (m, 1 H), 7.68 (s, 1 H), |
| | | | | 7.62-7.60 (m, 1 H), 7.29-7.25 (m, 3 H), 7.08-7.03 (m, 2 H), |
| | | | | 6.98 (d, J = 9.2 Hz, 1 H), 6.90 (s, 1 H), 6.77-6.62 |
| | | | | (m, 1 H), 6.05-6.00 (m, 1 H), 4.62 (t, J = 6.8 Hz, 2 H), |
| | | | | 4.37-4.28 (m, 1 H), 4.11-4.07 (m, 1 H), 3.82 (dd, J = 4.8, |
| | | | | 12.0 Hz, 1 H), 3.65-3.57 (m, 2 H), 3.51-3.48 |
| | | | | (m, 3 H), 3.09 (t, J = 7.2 Hz, 2 H), 3.00 (s, 6 H), 2.85-2.62 |
| | | | | (m, 5 H), 2.33-2.19 (m, 4 H), 2.04-1.99 (m, 4 H), |
| | | | | 1.97-1.85 (m, 4 H), 0.53-0.48 (m, 1 H), 0.13-0.02 |
| | | | | (m, 2 H), −0.24--0.40 (m, 2 H) |
| I-1446^{c} | TI | OP | 828.3 | 10.82 (s, 1 H), 10.76-10.76 (m, 1 H), 7.70 (d, J = 2.0 |
| | | | | Hz, 1 H), 7.59 (d, J = 1.6 Hz, 1 H), 7.50-7.37 (m, 5 H), |
| | | | | 7.10-6.96 (m, 4 H), 6.20-6.07 (m, 2 H), 4.36 (t, J = |
| | | | | 6.8 Hz, 2 H), 4.29-4.18 (m, 2 H), 3.82 (dd, J = 4.8, |
| | | | | 11.6 Hz, 1 H), 3.65-3.46 (m, 6 H), 3.00 (s, 6 H), |
| | | | | 2.98-2.90 (m, 2 H), 2.88-2.76 (m, 3 H), 2.73-2.61 (m, 1 H), |
| | | | | 2.38-2.26 (m, 2 H), 2.24-2.12 (m, 1 H), 2.07-1.83 |
| | | | | (m, 5 H), 0.52-0.38 (m, 1 H), 0.10-0.03 |
| | | | | (m, 2 H), −0.27--0.36 (m, 2 H) |
| I-1447 | TX | AR | 903.2 | 12.30-12.22 (m, 1 H), 11.07 (s, 1 H), 7.75-7.68 (m, |
| | | | | 1 H), 7.44-7.33 (m, 2 H), 7.32-7.26 (m, 1 H), 7.19 (t, |
| | | | | J = 8.0 Hz, 1 H), 7.10-7.01 (m, 1 H), 6.94 (d, J = 7.6 |
| | | | | Hz, 1 H), 6.88-6.80 (m, 1 H), 6.66 (s, 1 H), 6.22-6.12 |
| | | | | (m, 2 H), 5.28-5.17 (m, 1 H), 4.41-4.28 (m, 4 H), 3.64 |
| | | | | (t, J = 5.6 Hz, 1 H), 3.57 (t, J = 5.6 Hz, 1 H), 3.20-3.07 |
| | | | | (m, 6 H), 3.00-2.96 (m, 2 H), 2.91-2.84 (m, 4 H), |
| | | | | 2.68-2.58 (m, 5 H), 2.37-2.17 (m, 5 H), 2.01-1.92 |
| | | | | (m, 1 H), 1.87-1.77 (m, 4 H), 1.40 (d, J = 3.2 Hz, 6 H) |

ªThe cross coupling was performed under standard techniques from 30-80° C. for 1-3 hrs. Other catalysts systems could be utilized, such as Pd(dppf)Cl₂, and bases, such as K₃PO₄, and other halogens such as bromides and iodides. Final compounds were purified via standard techniques including prep-HPLC and other chromatography techniques.
^{b}LC-MS (ESI⁺) m/z reported as (M/2 + H) ⁺.
^{c}Bromide used for the coupling in place of chloride.
^{d}Pd(dppf)Cl2 used as the catalyst.
^{e}PEPPSI was used as the catalyst.
^{f}Otf used for the coupling in place of chloride.

TABLE 6
Compounds synthesized via Method 2, the cross coupling of the chlorides and boronic acids

| | LCMS | |
|---|---|---|
| | (ESI+) m/z | |
| I-# | (M + H)+ | ¹H NMR (400 MHz, DMSO-d₆) δ |
| I-149 | 806.2 | 10.82 (s, 1H), 8.15 (s, 1H), 8.11 (s, 1H), 7.69 (d, J = 2.8 Hz, 1H), 7.36-7.29 |
| | | (m, 1H), 7.23 (t, J = 8.0 Hz, 1H), 7.04 (s, 1H), 6.85-6.81 (m, 1H), 6.80-6.76 |
| | | (m, 2H), 6.52-6.43 (m, 1H), 6.25 (br d, J = 12.8 Hz, 1H), 4.63 (br t, J = 6.8 |
| | | Hz, 2H), 4.42-4.23 (m, 5H), 4.16 (br t, J = 6.8 Hz, 2H), 4.00 (br t, J = 6.4 Hz, |
| | | 2H), 3.94-3.76 (m, 3H), 3.64-3.52 (m, 3H), 3.49 (br s, 2H), 3.41-3.31 (m, |
| | | 3H), 3.16-3.06 (m, 3H), 3.01 (br s, 3H), 2.67-2.61 (m, 1H), 2.46 (br t, J = 6.8 |
| | | Hz, 2H), 2.37 (br d, J = 15.6 Hz, 4H), 2.31 (br s, 1H), 2.26-2.14 (m, 2H), 2.06- |
| | | 1.99 (m, 1H), 1.92-1.84 (m, 2H) |
| I-150 | 781.6 | 10.79 (s, 1H), 8.12 (s, 1H), 7.70 (d, J = 3.8 Hz, 1H), 7.37-7.30 (m, 1H), 7.13- |
| | | 7.03 (m, 1H), 6.86 (t, J = 9.4 Hz, 1H), 6.56-6.43 (m, 3H), 6.26 (br d, J = 13.4 |
| | | Hz, 1H), 5.88 (d, J = 7.8 Hz, 1H), 4.64 (br t, J = 6.8 Hz, 2H), 4.36-4.31 (m, |
| | | 4H), 4.21 (t, J = 6.8 Hz, 2H), 3.98-3.90 (m, 1H), 3.62 (br d, J = 4.0 Hz, 3H), |
| | | 3.56 (br t, J = 5.8 Hz, 1H), 3.49 (br s, 2H), 3.30 (s, 3H), 3.18-3.07 (m, 3H), |
| | | 3.03 (br s, 2H), 2.85 (br d, J = 16.6 Hz, 4H), 2.68 (br d, J = 1.6 Hz, 1H), 2.35- |
| | | 2.31 (m, 2H), 2.24 (br s, 1H), 2.09 (dt, J = 4.0, 8.6 Hz, 1H), 1.91-1.82 (m, 1H) |
| I-154 | 887.6 | 11.17-10.98 (m, 1H), 8.11 (s, 1H), 7.69 (d, J = 2.4 Hz, 1H), 7.38-7.27 (m, |
| | | 1H), 7.19-7.07 (m, 1H), 7.07-6.98 (m, 3H), 6.53-6.43 (m, 1H), 6.23 (br s, |
| | | 1H), 5.36 (br dd, J = 4.8, 12.0 Hz, 1H), 4.63 (br t, J = 6.8 Hz, 2H), 4.35-4.28 |
| | | (m, 4H), 4.17 (s, 2H), 4.14-4.08 (m, 1H), 3.89 (br d, J = 7.2 Hz, 2H), 3.60 (br |
| | | s, 2H), 3.57-3.50 (m, 5H), 3.26 (br s, 3H), 3.11 (br d, J = 16.0 Hz, 4H), 3.01 |
| | | (br s, 3H), 2.92-2.84 (m, 1H), 2.76-2.58 (m, 4H), 2.34-2.20 (m, 3H), 2.13- |
| | | 1.94 (m, 3H), 1.86-1.76 (m, 2H), 1.46-1.32 (m, 2H) |
| I-170 | 780.4 | 11.65-11.53 (m, 1H), 11.13 (br s, 1H), 8.16-8.09 (m, 1H), 7.73-7.65 (m, |
| | | 1H), 7.47 (s, 1H), 7.41 (br d, J = 7.2 Hz, 2H), 7.35-7.27 (m, 2H), 7.23 (t, J = |
| | | 9.2 Hz, 2H), 7.18-7.12 (m, 1H), 6.55-6.45 (m, 1H), 6.29 (br s, 1H), 5.42 (dd, |
| | | J = 5.2, 12.4 Hz, 1H), 4.67-4.60 (m, 2H), 4.44-4.34 (m, 2H), 3.83-3.76 (m, |
| | | 3H), 3.65-3.56 (m, 2H), 3.39 (s, 3H), 3.27-3.01 (m, 8H), 2.95-2.87 (m, 1H), |
| | | 2.73 (br dd, J = 4.0, 12.4 Hz, 1H), 2.68-2.61 (m, 1H), 2.36-2.24 (m, 2H), |
| | | 2.11-2.01 (m, 1H) |
| I-195 | 839.4 | 1.86 (br d, J = 11.6 Hz, 2 H) 1.92 (br s, 2 H) 1.94-2.07 (m, 2 H) 2.26-2.36 |
| | | (m, 2 H) 2.59-2.71 (m, 2 H) 2.72-2.82 (m, 2 H) 2.83-2.89 (m, 2 H) 2.90- |
| | | 2.94 (m, 1 H) 3.06-3.20 (m, 6 H) 3.36 (s, 3 H) 3.57-3.66 (m, 2 H) 3.72-3.75 |
| | | (m, 3 H) 3.96 (br d, J = 12.0 Hz, 2 H) 4.36-4.43 (m, 2 H) 4.62-4.67 (m, 2 H) |
| | | 5.36 (dd, J = 12.8, 5.2 Hz, 1 H) 6.26 (br d, J = 3.6 Hz, 1 H) 6.50-6.54 (m, 1 |
| | | H) 6.67 (br d, J = 8.0 Hz, 1 H) 6.73 (s, 1 H) 6.97-7.01 (m, 1 H) 7.03-7.07 |
| | | (m, 1 H) 7.07-7.10 (m, 1 H) 7.17 (s, 1 H) 7.22 (d, J = 8.4 Hz, 1 H) 7.32-7.37 |
| | | (m, 1 H) 7.66-7.74 (m, 1 H) 8.10-8.17 (m, 1 H) 11.10 (s, 1 H) 11.52 (s, 1 H) |
| I-198 | 717.5 | 12.10 (s, 1H), 11.15 (s, 1H), 7.44 (d, J = 7.6 Hz, 1H), 7.33 (s, 1H), 7.29 (t, J = |
| | | 8.0 Hz, 2H), 7.23 (d, J = 7.6 Hz, 1H), 7.13-7.07 (m, 1H), 7.02-6.93 (m, 1H), |
| | | 6.52 (s, 1H), 6.18-6.12 (m, 1H), 5.47-5.41 (m, 1H), 4.37-4.32 (m, 2H), 3.80 |
| | | (d, J = 4.4 Hz, 6H), 3.65-3.59 (m, 2H), 3.22-3.01 (m, 6H), 2.93-2.86 (m, |
| | | 1H), 2.80-2.72 (m, 1H), 2.67 (s, 1H), 2.33 (s, 1H), 2.27 (d, J = 4.0 Hz, 1H), |
| | | 2.09 (d, J = 6.4 Hz, 3H), 2.07-2.02 (m, 1H) |
| I-236 | 798.3 | 12.21-12.01 (m, 1H), 11.26-11.04 (m, 1H), 8.19-8.07 (m, 1H), 7.79-7.66 |
| | | (m, 1H), 7.48-7.41 (m, 1H), 7.36-7.20 (m, 4H), 7.16-7.07 (m, 1H), 7.04- |
| | | 6.92 (m, 1H), 6.52 (br s, 1H), 6.24-6.09 (m, 1H), 5.52-5.40 (m, 1H), 4.71- |
| | | 4.60 (m, 2H), 4.44-4.28 (m, 2H), 3.84-3.76 (m, 6H), 3.67-3.59 (m, 2H), |
| | | 3.21-3.07 (m, 6H), 2.93-2.87 (m, 1H), 2.71-2.63 (m, 3H), 2.44-2.21 (m, |
| | | 3H), 2.14-2.01 (m, 1H) |
| I-244 | 687.5 | 7.83-7.68 (m, 4H), 7.24 (br dd, J = 8.0, 18.0 Hz, 2H), 7.17-7.06 (m, 2H), |
| | | 6.92 (br s, 1H), 6.19 (br s, 1H), 5.44 (br dd, J = 5.6, 13.2 Hz, 1H), 4.37 (br s, |
| | | 2H), 3.79 (s, 3H), 3.67-3.58 (m, 2H), 3.25-3.02 (m, 6H), 2.97-2.87 (m, 1H), |
| | | 2.80-2.71 (m, 1H), 2.70-2.61 (m, 2H), 2.41 (br d, J = 10.4 Hz, 2H), 2.32- |
| | | 2.24 (m, 1H), 2.09 (br d, J = 6.4 Hz, 4H) |
| I-248 | 768.3 | 12.27 (br s, 1H), 11.18-11.10 (m, 1H), 8.15-8.08 (m, 1H), 7.80-7.75 (m, |
| | | 2H), 7.74-7.67 (m, 3H), 7.26 (d, J = 7.6 Hz, 1H), 7.24-7.20 (m, 1H), 7.16- |
| | | 7.07 (m, 2H), 6.92-6.90 (m, 1H), 6.23-6.15 (m, 1H), 5.47-5.40 (m, 1H), |
| | | 4.66-4.60 (m, 2H), 4.41-4.33 (m, 2H), 3.78 (s, 3H), 3.66 (br t, J = 5.6 Hz, |
| | | 1H), 3.63-3.59 (m, 1H), 3.27-3.15 (m, 3H), 3.14-3.03 (m, 5H), 2.96-2.87 |
| | | (m, 1H), 2.80-2.71 (m, 1H), 2.68-2.62 (m, 1H), 2.37-2.24 (m, 2H), 2.10- |
| | | 2.01 (m, 1H) |
| I-258 | 774.6 | 12.11 (s, 1H), 11.08 (s, 1H), 7.56-7.45 (m, 2H), 7.07-7.01 (m, 3H), 7.01- |
| | | 6.96 (m, 1H), 6.95-6.80 (m, 3H), 6.20-6.09 (m, 1H), 5.34 (dd, J = 5.2, 12.4 |
| | | Hz, 1H), 4.34 (d, J = 8.4 Hz, 2H), 3.77 (d, J = 12.0 Hz, 2H), 3.66-3.57 (m, |
| | | 2H), 3.33-3.33 (m, 3H), 3.23-3.00 (m, 6H), 2.94-2.85 (m, 1H), 2.76-2.61 |
| | | (m, 6H), 2.37 (s, 1H), 2.26 (s, 1H), 2.08 (d, J = 7.6 Hz, 3H), 2.04-1.97 (m, 1H), |
| | | 1.84 (d, J = 12.0 Hz, 2H), 1.64-1.53 (m, 2H), 1.46 (s, 1H), 1.36-1.25 (m, 2H) |
| I-265 | 776.6 | 12.05-11.84 (m, 1H), 11.17-10.92 (m, 1H), 7.27-7.13 (m, 2H), 7.08-6.96 |
| | | (m, 2H), 6.93-6.83 (m, 1H), 6.72 (s, 1H), 6.66 (br d, J = 8.4 Hz, 1H), 6.51 (br |
| | | s, 1H), 6.16-6.09 (m, 1H), 5.42-5.25 (m, 1H), 4.37-4.25 (m, 2H), 4.02- |
| | | 3.90 (m, 2H), 3.74 (s, 3H), 3.68-3.58 (m, 2H), 3.36 (s, 3H), 3.22-3.15 (m, |
| | | 2H), 3.12-3.05 (m, 2H), 2.98-2.74 (m, 5H), 2.73-2.61 (m, 2H), 2.42-2.34 |
| | | (m, 2H), 2.30-2.24 (m, 1H), 2.12-2.06 (m, 3H), 2.06-1.98 (m, 1H), 1.97- |
| | | 1.78 (m, 4H) |
| I-267 | 772.5 | 12.36-11.74 (m, 1H), 11.44-10.92 (m, 1H), 8.16-8.05 (m, 1H), 7.74-7.65 |
| | | (m, 1H), 7.64-7.56 (m, 2H), 7.44 (br d, J = 8.0 Hz, 2H), 7.08-6.97 (m, 4H), |
| | | 6.86 (d, J = 2.4 Hz, 1H), 6.16 (br s, 1H), 5.44-5.29 (m, 1H), 4.68-4.57 (m, |
| | | 2H), 4.42-4.28 (m, 2H), 3.70-3.59 (m, 5H), 3.32 (s, 6H), 3.29-3.24 (m, 2H), |
| | | 3.10 (br d, J = 7.2 Hz, 2H), 3.01 (br d, J = 9.2 Hz, 2H), 2.95-2.86 (m, 1H), |
| | | 2.78-2.69 (m, 1H), 2.67-2.61 (m, 1H), 2.38-2.23 (m, 2H), 2.06-1.95 (m, |
| | | 1H)) |
| I-278 | 778.5 | 11.86 (br s, 1H), 11.10 (s, 1H), 7.20-7.16 (m, 2H), 7.07-7.03 (m, 1H), 7.01- |
| | | 6.97 (m, 1H), 6.90 (dd, J = 6.0, 10.0 Hz, 1H), 6.71 (s, 1H), 6.66 (br d, J = 8.4 |
| | | Hz, 1H), 6.49 (t, J = 2.4 Hz, 1H), 5.36 (dd, J = 5.2, 12.8 Hz, 1H), 4.54-4.42 |
| | | (m, 1H), 3.95 (br d, J = 12.0 Hz, 2H), 3.90-3.83 (m, 1H), 3.73 (s, 3H), 3.36 (s, |
| | | 3H), 3.31-3.27 (m, 1H), 3.26-3.19 (m, 2H), 3.10 (dt, J = 2.4, 11.2 Hz, 4H), |
| | | 2.99-2.94 (m, 1H), 2.92-2.83 (m, 3H), 2.77 (br s, 1H), 2.75-2.68 (m, 2H), |
| | | 2.66 (br d, J = 5.2 Hz, 1H), 2.62-2.57 (m, 1H), 2.04 (d, J = 8.8 Hz, 3H), 1.91 |
| | | (br s, 4H), 1.89-1.76 (m, 4H) |
| I-301 | 723.3 | 12.16 (br s, 1H), 11.15 (s, 1H), 7.80 (s, 1H), 7.64-7.59 (m, 1H), 7.57-7.53 |
| | | (m, 1H), 7.49 (d, J = 1.2 Hz, 1H), 7.34 (dd, J = 1.6, 8.0 Hz, 1H), 7.24 (d, J = 8.0 |
| | | Hz, 1H), 7.06 (dd, J = 5.6, 11.6 Hz, 1H), 6.49 (br s, 1H), 5.44 (dd, J = 5.6, 12.8 |
| | | Hz, 1H), 4.48 (br t, J = 10.0 Hz, 1H), 3.92-3.83 (m, 1H), 3.40 (s, 3H), 3.19- |
| | | 3.01 (m, 7H), 2.96-2.87 (m, 1H), 2.79-2.62 (m, 3H), 2.08-2.02 (m, 4H), |
| | | 1.96-1.74 (m, 3H), 1.67-1.42 (m, 1H) |
| I-305 | 811.3 | 12.03 (br s, 1H), 11.23-10.96 (m, 1H), 7.30 (dd, J = 2.4, 8.4 Hz, 1H), 7.14- |
| | | 7.07 (m, 2H), 7.07-6.98 (m, 2H), 6.97-6.88 (m, 2H), 6.45 (br s, 1H), 5.35 |
| | | (dd, J = 5.2, 12.8 Hz, 1H), 4.54-4.41 (m, 1H), 3.86 (br d, J = 12.8 Hz, 1H), |
| | | 3.34 (s, 3H), 3.25 (br d, J = 4.4 Hz, 4H), 3.21-2.95 (m, 8H), 2.92-2.79 (m, |
| | | 3H), 2.78-2.67 (m, 2H), 2.67-2.53 (m, 8H), 2.04 (d, J = 8.0 Hz, 3H), 1.96- |
| | | 1.73 (m, 3H), 1.68-1.39 (m, 1H) |
| I-311 | 733.5 | 12.03-11.93 (m, 1H), 11.18-11.07 (m, 1H), 7.46 (d, J = 1.2 Hz, 1H), 7.40 (s, |
| | | 1H), 7.34-7.29 (m, 1H), 7.26 (s, 1H), 7.22 (br s, 2H), 7.11-7.06 (m, 1H), 6.60- |
| | | 6.52 (m, 1H), 5.49-5.36 (m, 1H), 4.53-4.44 (m, 1H), 4.10 (br d, J = 6.8 Hz, |
| | | 2H), 3.91-3.83 (m, 1H), 3.39 (s, 3H), 3.19-2.99 (m, 7H), 2.95-2.86 (m, 1H), |
| | | 2.79-2.54 (m, 4H), 2.04 (d, J = 8.4 Hz, 4H), 1.96-1.74 (m, 3H), 1.66-1.42 |
| | | (m, 1H), 1.20 (t, J = 6.8 Hz, 3H) |
| I-328 | 778.4 | 11.92 (br s, 1H), 11.30-10.78 (m, 1H), 7.27 (d, J = 7.6 Hz, 1H), 7.07 (s, 1H), |
| | | 7.03-6.87 (m, 4H), 6.72 (dd, J = 2.0, 8.8 Hz, 1H), 6.48 (d, J = 2.8 Hz, 1H), |
| | | 5.31 (dd, J = 5.2, 12.8 Hz, 1H), 4.48 (br t, J = 10.0 Hz, 1H), 3.86 (br d, J = 13.2 |
| | | Hz, 1H), 3.80-3.70 (m, 5H), 3.34-3.33 (m, 2H), 3.28-3.04 (m, 7H), 3.00 (br |
| | | d, J = 3.2 Hz, 2H), 2.82-2.70 (m, 4H), 2.69 (br s, 3H), 2.04 (d, J = 8.8 Hz, 3H), |
| | | 2.01-1.72 (m, 8H), 1.66-1.38 (m, 1H) |
| I-331 | 719.6 | 11.99 (br s, 1H), 11.15 (s, 1H), 7.41 (d, J = 7.6 Hz, 1H), 7.36-7.19 (m, 4H), |
| | | 7.15-7.08 (m, 1H), 7.00 (dd, J = 5.6, 10.4 Hz, 1H), 6.51 (br s, 1H), 5.44 (dd, J = |
| | | 5.6, 12.8 Hz, 1H), 4.48 (br t, J = 11.6 Hz, 1H), 3.87 (br d, J = 12.8 Hz, 1H), |
| | | 3.80 (d, J = 2.0 Hz, 5H), 3.26-2.92 (m, 9H), 2.83-2.54 (m, 4H), 2.08 (br s, |
| | | 1H), 2.04 (d, J = 7.6 Hz, 3H), 1.97-1.74 (m, 3H), 1.66-1.41 (m, 1H) |
| I-334 | 807.4 | 11.90 (br s, 1H), 11.14-11.03 (m, 1H), 7.23 (d, J = 7.6 Hz, 1H), 7.06 (s, 1H), |
| | | 7.03-6.88 (m, 5H), 6.53-6.42 (m, 1H), 5.41-5.30 (m, 1H), 4.54-4.39 (m, |
| | | 1H), 3.92-3.82 (m, 1H), 3.75-3.62 (m, 6H), 3.27-3.15 (m, 3H), 3.14-2.92 |
| | | (m, 11H), 2.91-2.82 (m, 4H), 2.57 (br s, 6H), 2.03 (br d, J = 8.8 Hz, 3H), 2.00- |
| | | 1.88 (m, 2H), 1.88-1.80 (m, 1H), 1.79-1.71 (m, 1H), 1.65-1.40 (m, 1H) |
| I-369 | 798.3 | 11.34-10.94 (m, 1H), 7.74-7.66 (m, 1H), 7.55-7.49 (m, 2H), 7.45-7.40 |
| | | (m, 1H), 7.36 (br s, 2H), 7.32 (br d, J = 6.8 Hz, 1H), 7.27 (br d, J = 7.2 Hz, 1H), |
| | | 7.23 (br d, J = 7.6 Hz, 1H), 7.14-7.06 (m, 1H), 6.24-6.08 (m, 2H), 5.48- |
| | | 5.38 (m, 1H), 4.39-4.33 (m, 3H), 4.28 (br s, 1H), 3.79 (br d, J = 9.6 Hz, 6H), |
| | | 3.66-3.56 (m, 2H), 3.21 (br s, 3H), 3.00 (br s, 3H), 2.97-2.86 (m, 2H), 2.83- |
| | | 2.58 (m, 3H), 2.33 (br s, 1H), 2.27 (br dd, J = 1.6, 3.2 Hz, 1H), 2.11-2.01 (m, |
| | | 1H) |
| I-379 | 742.3 | 11.96-11.74 (m, 1H), 10.97-10.77 (m, 1H), 7.39 (s, 1H), 7.33-7.25 (m, 2H), |
| | | 7.21-7.12 (m, 1H), 6.90 (dd, J = 5.9, 10.0 Hz, 1H), 6.71 (s, 1H), 6.68-6.62 |
| | | (m, 1H), 6.65 (br d, J = 8.4 Hz, 1H), 6.53-6.45 (m, 1H), 4.54-4.41 (m, 1H), |
| | | 4.19 (dd, J = 5.2, 12.4 Hz, 1H), 4.01-3.82 (m, 3H), 3.73 (s, 3H), 3.27-2.93 |
| | | (m, 8H), 2.90-2.65 (m, 5H), 2.55 (br d, J = 3.6 Hz, 1H), 2.37-2.21 (m, 1H), |
| | | 2.06-2.01 (m, 3H), 2.00-1.89 (m, 4H), 1.88-1.66 (m, 4H), 1.64-1.40 (m, |
| | | 1H) |
| I-387 | 703.4 | 11.60-11.54 (m, 1H), 11.32-10.85 (m, 1H), 7.42-7.38 (m, 1H), 7.32-7.27 |
| | | (m, 1H), 7.12-7.08 (m, 1H), 7.06-6.99 (m, 5H), 6.47 (d, J = 1.2 Hz, 1H), 6.28 |
| | | (s, 1H), 5.40 (dd, J = 5.6, 12.8 Hz, 1H), 4.39 (s, 2H), 3.69 (s, 3H), 3.62 (s, 3H), |
| | | 3.61-3.55 (m, 2H), 3.33 (s, 6H), 3.31-3.26 (m, 2H), 3.03-2.99 (m, 2H), 2.94- |
| | | 2.87 (m, 1H), 2.78-2.71 (m, 1H), 2.66-2.60 (m, 1H), 2.38-2.25 (m, 2H), |
| | | 2.13-2.09 (m, 3H), 2.05-1.97 (m, 1H) |
| I-391 | 855.4 | 11.10 (s, 1H), 8.42 (s, 1H), 7.77-7.68 (m, 3H), 7.55-7.51 (m, 1H), 7.48-7.43 |
| | | (m, 1H), 7.38 (d, J = 1.2 Hz, 1H), 7.09 (d, J = 8.8 Hz, 2H), 6.99-6.95 (m, 2H), |
| | | 6.92-6.88 (m, 1H), 6.24-6.13 (m, 2H), 5.41-5.33 (m, 1H), 4.40-4.33 (m, |
| | | 3H), 4.30 (s, 1H), 3.85 (d, J = 12.4 Hz, 2H), 3.66 (t, J = 6.0 Hz, 1H), 3.59 (s, |
| | | 3H), 3.29 (s, 6H), 3.05 (s, 2H), 3.00-2.94 (m, 4H), 2.92-2.85 (m, 1H), 2.79- |
| | | 2.72 (m, 2H), 2.70-2.67 (m, 1H), 2.66-2.59 (m, 1H), 2.28 (s, 1H), 2.05-1.96 |
| | | (m, 1H), 1.94-1.85 (m, 2H), 1.60 (t, J = 6.4 Hz, 2H), 1.40-1.30 (m, 2H) |
| I-400 | 744.3 | 11.64-11.57 (m, 1H), 11.00-10.93 (m, 1H), 8.54-8.45 (m, 1H), 8.15-8.11 |
| | | (m, 1H), 7.75-7.67 (m, 2H), 7.57 (d, J = 8.0 Hz, 1H), 7.46-7.41 (m, 3H), 7.35 |
| | | (s, 1H), 7.28 (dd, J = 1.2, 7.6 Hz, 1H), 7.18-7.14 (m, 1H), 6.52-6.49 (m, 1H), |
| | | 6.30 (s, 1H), 4.67-4.62 (m, 2H), 4.43-4.37 (m, 2H), 4.31-4.27 (m, 1H), 3.81- |
| | | 3.78 (m, 3H), 3.65-3.58 (m, 2H), 3.19-3.06 (m, 6H), 2.85-2.77 (m, 1H), |
| | | 2.61-2.56 (m, 1H), 2.37-2.32 (m, 3H), 2.31-2.25 (m, 1H), 2.06-2.00 (m, |
| | | 1H) |
| I-404 | 805.4 | 11.20-10.94 (m, 1H), 7.52-7.46 (m, 1H), 7.36-7.20 (m, 2H), 7.00-6.95 |
| | | (m, 2H), 6.93-6.89 (m, 1H), 6.69-6.60 (m, 2H), 6.18-6.08 (m, 1H), 5.42- |
| | | 5.33 (m, 1H), 4.31 (d, J = 2.8 Hz, 2H), 3.86 (d, J = 12.4 Hz, 2H), 3.73 (s, 3H), |
| | | 3.65-3.61 (m, 1H), 3.60 (s, 3H), 3.22 (s, 3H), 3.03-2.98 (m, 3H), 2.96 (s, 1H), |
| | | 2.92-2.85 (m, 1H), 2.81-2.69 (m, 3H), 2.68-2.59 (m, 2H), 2.38 (s, 2H), 2.27 |
| | | (d, J = 3.2 Hz, 1H), 2.08 (d, J = 8.0 Hz, 3H), 2.03-1.99 (m, 1H), 1.90 (d, J = |
| | | 12.4 Hz, 2H), 1.66-1.55 (m, 3H), 1.43-1.31 (m, 2H) |
| I-418 | 782.3 | 11.85 (s, 1H), 10.51 (s, 1H), 7.59 (dd, J = 1.6, 7.6 Hz, 1H), 7.53 (dd, J = 1.6, 8.0 |
| | | Hz, 1H), 7.46-7.39 (m, 1H), 7.16 (d, J = 8.4 Hz, 1H), 6.88 (dd, J = 6.0, 10.8 |
| | | Hz, 1H), 6.69 (d, J = 1.6 Hz, 1H), 6.61 (d, J = 8.4 Hz, 1H), 6.47 (s, 1H), 4.53- |
| | | 4.41 (m, 1H), 3.86 (d, J = 12.4 Hz, 1H), 3.79-3.66 (m, 6H), 3.61 (td, J = 6.4, |
| | | 12.4 Hz, 1H), 3.29 (s, 4H), 3.24-2.92 (m, 8H), 2.81-2.71 (m, 6H), 2.70-2.56 |
| | | (m, 1H), 2.04 (d, J = 9.2 Hz, 3H), 1.92 (s, 1H), 1.88-1.71 (m, 2H), 1.66-1.40 |
| | | (m, 1H) |
| I-425 | 841.4 | 11.17-11.02 (m, 1H), 7.59-7.51 (m, 1H), 7.50-7.36 (m, 2H), 7.34-7.30 (m, |
| | | 1H), 7.19-7.13 (m, 1H), 7.00-6.94 (m, 3H), 6.93-6.88 (m, 1H), 6.81 (d, J = |
| | | 1.6 Hz, 1H), 6.21-6.09 (m, 1H), 5.42-5.32 (m, 1H), 4.32 (s, 2H), 3.86 (d, J = |
| | | 12.4 Hz, 2H), 3.66-3.57 (m, 5H), 3.22 (s, 3H), 3.03-2.95 (m, 4H), 2.87-2.72 |
| | | (m, 3H), 2.71-2.60 (m, 2H), 2.38 (s, 1H), 2.27 (d, J = 4.0 Hz, 1H), 2.08 (d, J = |
| | | 9.2 Hz, 3H), 2.04-1.97 (m, 1H), 1.90 (d, J = 12.0 Hz, 2H), 1.60 (d, J = 6.8 Hz, |
| | | 3H), 1.40-1.26 (m, 2H) |
| I-426 | 921.3 | 11.10 (s, 1H), 7.78-7.65 (m, 1H), 7.53 (s, 1H), 7.46-7.28 (m, 3H), 7.20-7.13 |
| | | (m, 1H), 7.03-6.86 (m, 4H), 6.81 (s, 1H), 6.22-6.17 (m, 1H), 6.14 (d, J = 11.2 |
| | | Hz, 1H), 5.43-5.30 (m, 1H), 4.41-4.31 (m, 3H), 4.28 (s, 1H), 3.86 (d, J = 12.0 |
| | | Hz, 2H), 3.65 (t, J = 5.6 Hz, 1H), 3.59 (s, 3H), 3.58-3.56 (m, 1H), 3.21 (s, 2H), |
| | | 3.00 (d, J = 7.2 Hz, 4H), 2.98-2.91 (m, 3H), 2.91-2.76 (m, 3H), 2.76-2.67 |
| | | (m, 2H), 2.65 (d, J = 4.4 Hz, 1H), 2.61 (d, J = 2.4 Hz, 1H), 2.27 (d, J = 1.6 Hz, |
| | | 1H), 2.06-1.96 (m, 1H), 1.90 (d, J = 11.6 Hz, 2H), 1.60 (d, J = 7.6 Hz, 3H), |
| | | 1.40-1.28 (m, 2H) |
| I-439 | 806.5 | 12.10 (s, 1H), 11.09 (s, 1H), 7.55-7.44 (m, 2H), 7.03 (d, J = 8.8 Hz, 2H), 7.00- |
| | | 6.97 (m, 1H), 6.87-6.82 (m, 1H), 6.95-6.79 (m, 2H), 6.14 (d, J = 2.0 Hz, |
| | | 1H), 5.37 (dd, J = 5.6, 12.8 Hz, 1H), 4.34 (d, J = 6.8 Hz, 2H), 3.76 (d, J = 12.4 |
| | | Hz, 2H), 3.66-3.58 (m, 2H), 3.56 (s, 3H), 3.24-2.98 (m, 6H), 2.94-2.83 (m, |
| | | 3H), 2.75-2.59 (m, 4H), 2.37 (d, J = 3.2 Hz, 1H), 2.26 (d, J = 1.6 Hz, 1H), 2.08 |
| | | (d, J = 8.0 Hz, 3H), 2.01 (dd, J = 5.2, 10.8 Hz, 1H), 1.78 (d, J = 12.0 Hz, 2H), |
| | | 1.70-1.57 (m, 2H), 1.51-1.34 (m, 3H), 1.30-1.21 (m, 2H) |
| I-446 | 840.4 | 12.07 (s, 1H), 11.09 (s, 1H), 7.37-7.32 (m, 1H), 7.32 (s, 1H), 6.98 (d, J = 4.8 |
| | | Hz, 2H), 6.96-6.93 (m, 1H), 6.92-6.87 (m, 2H), 6.80 (s, 1H), 6.55 (d, J = 2.8 |
| | | Hz, 1H), 6.17-6.10 (m, 1H), 5.38 (dd, J = 5.4, 12.4 Hz, 1H), 4.36-4.30 (m, |
| | | 2H), 3.83 (d, J = 12.4 Hz, 2H), 3.65-3.61 (m, 1H), 3.60 (s, 3H), 3.24-3.01 |
| | | (m, 6H), 3.00-2.95 (m, 2H), 2.92-2.84 (m, 1H), 2.84-2.72 (m, 3H), 2.71- |
| | | 2.58 (m, 2H), 2.38 (d, J = 3.2 Hz, 1H), 2.27 (s, 1H), 2.08 (d, J = 9.2 Hz, 3H), |
| | | 2.03-1.98 (m, 1H), 1.91 (d, J = 12.4 Hz, 2H), 1.61 (t, J = 6.0 Hz, 3H), 1.40- |
| | | 1.31 (m, 2H) |
| I-447 | 842.4 | 11.98 (s, 1H), 11.10 (s, 1H), 7.34-7.13 (m, 2H), 6.98 (s, 2H), 6.95-6.88 (m, |
| | | 3H), 6.80 (s, 1H), 6.55 (s, 1H), 5.41-5.34 (m, 1H), 4.52-4.45 (m, 1H), 3.83 |
| | | (d, J = 12.4 Hz, 3H), 3.60 (s, 3H), 3.18-3.06 (m, 6H), 3.00-2.95 (m, 3H), 2.93- |
| | | 2.85 (m, 2H), 2.78 (t, J = 11.2 Hz, 3H), 2.69-2.60 (m, 3H), 2.04 (d, J = 10.4 |
| | | Hz, 3H), 1.91 (d, J = 11.2 Hz, 3H), 1.84-1.75 (m, 1H), 1.75-1.74 (m, 1H), |
| | | 1.61 (s, 3H), 1.39-1.32 (m, 2H), 1.25 (s, 1H) |
| I-451 | 821.1 | 10.86 (s, 1H), 7.83-7.64 (m, 3H), 7.54 (s, 1H), 7.53-7.45 (m, 1H), 7.45-7.35 |
| | | (m, 1H), 7.13 (d, J = 8.4 Hz, 2H), 6.68 (d, J = 8.0 Hz, 1H), 6.59 (d, J = 14.4 Hz, |
| | | 1H), 6.23-6.11 (m, 2H), 5.14 (d, J = 6.4 Hz, 1H), 4.37 (q, J = 6.8 Hz, 3H), 4.32- |
| | | 4.23 (m, 2H), 3.81 (s, 3H), 3.65 (t, J = 5.6 Hz, 1H), 3.59 (t, J = 5.6 Hz, 1H), |
| | | 3.37 (s, 1H), 3.28 (s, 6H), 3.08 (d, J = 4.4 Hz, 4H), 3.04 (s, 2H), 3.02-2.90 (m, |
| | | 3H), 2.81 (ddd, J = 5.2, 13.2, 18.0 Hz, 1H), 2.59-2.51 (m, 1H), 2.37-2.23 (m, |
| | | 2H), 2.21-2.08 (m, 1H), 2.02-1.81 (m, 1H) |
| I-453 | 776.3 | 10.81 (s, 1H), 7.81-7.74 (m, 2H), 7.71 (dd, J = 2.0, 10.4 Hz, 1H), 7.54 (s, 1H), |
| | | 7.53-7.46 (m, 1H), 7.44-7.34 (m, 1H), 7.15 (d, J = 8.8 Hz, 2H), 7.10-7.02 |
| | | (m, 2H), 7.02-6.97 (m, 1H), 6.23-6.09 (m, 2H), 4.42-4.26 (m, 4H), 3.82 |
| | | (dd, J = 4.8, 11.6 Hz, 1H), 3.69-3.53 (m, 2H), 3.44-3.35 (m, 4H), 3.28-3.11 |
| | | (m, 8H), 3.10-3.00 (m, 3H), 3.00-2.92 (m, 2H), 2.70-2.60 (m, 1H), 2.36- |
| | | 2.31 (m, 1H), 2.30-2.14 (m, 2H), 2.07-1.94 (m, 1H) |
| I-456 | 761.3 | 12.14-12.04 (m, 1H), 11.15-11.05 (m, 1H), 7.18-7.16 (m, 1H), 7.04-6.93 |
| | | (m, 5 H), 6.90-6.80 (m, 1H), 6.40 (s, 1H), 6.20-6.10 (m, 1H), 5.40-5.37 (m, 1H), |
| | | 4.40-4.21 (m, 2H), 3.92-3.75 (m, 2H), 3.68 (s, 3H), 3.65-3.55 (m, 3H), 3.20- |
| | | 3.14 (m, 3H), 3.03-2.78 (m, 8H), 2.75-2.60 (m, 2H), 2.40-2.32 (m, 2H), 2.30- |
| | | 2.23 (m, 1H), 2.16-2.10 (m, 3H), 2.10-1.98 (m, 4H) |
| I-461 | 788.4 | 12.16-12.02 (m, 1H), 11.16-10.99 (m, 1H), 7.53-7.45 (m, 2H), 7.03 (d, J = |
| | | 8.8 Hz, 2H), 6.99-6.95 (m, 2H), 6.94-6.87 (m, 2H), 6.85 (d, J = 2.4 Hz, 1H), |
| | | 6.14 (s, 1H), 5.36 (dd, J = 5.2, 12.4 Hz, 1H), 4.34 (d, J = 6.4 Hz, 2H), 3.76 (d, J = |
| | | 12.0 Hz, 2H), 3.65-3.58 (m, 2H), 3.56 (s, 3H), 3.24-2.97 (m, 6H), 2.94- |
| | | 2.84 (m, 3H), 2.75-2.60 (m, 4H), 2.37 (d, J = 2.8 Hz, 1H), 2.26 (d, J = 3.2 Hz, |
| | | 1H), 2.08 (d, J = 8.0 Hz, 3H), 2.03-1.96 (m, 1H), 1.84-1.73 (m, 2H), 1.72- |
| | | 1.60 (m, 2H), 1.50-1.35 (m, 3H), 1.30-1.21 (m, 2H) |
| I-464 | 748.4 | 11.95 (s, 1H), 11.10 (s, 1H), 7.28 (s, 1H), 7.20-7.13 (m, 1H), 7.13-7.05 (m, |
| | | 2H), 6.89-6.80 (m, 1H), 6.48 (d, J = 2.4 Hz, 1H), 6.24 (d, J = 1.6 Hz, 1H), 6.19 |
| | | (d, J = 8.0 Hz, 1H), 6.13-6.08 (m, 1H), 5.37 (dd, J = 5.2, 12.8 Hz, 1H), 4.41- |
| | | 4.23 (m, 4H), 4.09-3.99 (m, 1H), 3.92 (t, J = 6.4 Hz, 2H), 3.71 (s, 3H), 3.65- |
| | | 3.57 (m, 2H), 3.36 (s, 3H), 3.25-2.98 (m, 6H), 2.95-2.86 (m, 1H), 2.77-2.58 |
| | | (m, 2H), 2.39-2.24 (m, 2H), 2.11-2.04 (m, 3H), 2.04-1.98 (m, 1H) |
| I-476 | 859.4 | 12.05 (br d, J = 1.6 Hz, 1H), 11.30-10.93 (m, 1H), 7.17 (d, J = 8.4 Hz, 1H), |
| | | 7.01-6.91 (m, 1H), 6.84 (d, J = 6.4 Hz, 1H), 6.76-6.66 (m, 3H), 6.65-6.58 |
| | | (m, 1H), 6.49 (d, J = 3.2 Hz, 1H), 6.14-6.03 (m, 1H), 5.38 (s, 1H), 4.81-4.60 |
| | | (m, 4H), 4.39-4.36 (m, 1H), 4.26-4.18 (m, 1H), 4.04 (br s, 1H), 3.72 (s, 3H), |
| | | 3.68 (s, 4H), 3.60 (s, 3H), 3.19-2.98 (m, 6H), 2.92-2.84 (m, 1H), 2.72-2.59 |
| | | (m, 2H), 2.30 (br s, 2H), 2.04-1.97 (m, 1H), 1.94 (br s, 4H) |
| I-477 | 822.0 | 12.03 (br s, 1H), 10.86 (s, 1H), 7.71 (dd, J = 2.0, 10.8 Hz, 1H), 7.54 (d, J = 8.8 |
| | | Hz, 2H), 7.41 (dd, J = 1.2, 16.0 Hz, 1H), 7.11 (br d, J = 8.8 Hz, 2H), 7.00 (dd, J = |
| | | 2.0, 5.2 Hz, 1H), 6.85 (br s, 1H), 6.68 (d, J = 8.0 Hz, 1H), 6.59 (d, J = 14.4 |
| | | Hz, 1H), 6.20 (td, J = 2.0, 10.4 Hz, 1H), 5.13 (d, J = 6.4 Hz, 1H), 4.49 (br d, J = |
| | | 12.4 Hz, 1H), 4.38-4.33 (m, 2H), 4.31-4.25 (m, 1H), 3.89 (br d, J = 13.6 |
| | | Hz, 1H), 3.82 (s, 3H), 3.35 (br d, J = 5.2 Hz, 6H), 3.21-3.15 (m, 2H), 3.10 (br |
| | | d, J = 4.4 Hz, 6H), 2.93 (td, J = 7.2, 14.4 Hz, 3H), 2.86-2.75 (m, 2H), 2.57 (br |
| | | s, 2H), 2.17-2.11 (m, 1H), 1.95-1.88 (m, 3H), 1.76 (br d, J = 10.8 Hz, 1H), |
| | | 1.55-1.43 (m, 1H) |
| I-478 | 822.0 | 12.03 (br s, 1H), 10.86 (s, 1H), 7.71 (dd, J = 2.0, 10.8 Hz, 1H), 7.54 (d, J = 8.8 |
| | | Hz, 2H), 7.41 (dd, J = 1.6, 16.0 Hz, 1H), 7.11 (br d, J = 8.4 Hz, 2H), 7.00 (br d, |
| | | J = 3.6 Hz, 1H), 6.85 (br s, 1H), 6.68 (d, J = 8.0 Hz, 1H), 6.59 (d, J = 14.4 Hz, |
| | | 1H), 6.20 (td, J = 2.0, 10.4 Hz, 1H), 5.14 (d, J = 6.4 Hz, 1H), 4.49 (br d, J = 10.8 |
| | | Hz, 1H), 4.38-4.34 (m, 2H), 4.28 (br t, J = 6.0 Hz, 1H), 3.91-3.87 (m, 1H), |
| | | 3.82 (s, 3H), 3.35 (br d, J = 4.8 Hz, 6H), 3.21-3.15 (m, 2H), 3.10 (br d, J = 4.0 |
| | | Hz, 6H), 2.97-2.89 (m, 3H), 2.84-2.76 (m, 2H), 2.57 (br s, 2H), 2.18-2.11 |
| | | (m, 1H), 1.96-1.87 (m, 3H), 1.80-1.74 (m, 1H), 1.57-1.41 (m, 1H) |
| I-485 | 750.2 | 11.82 (s, 1H), 11.09 (s, 1H), 7.27 (s, 1H), 7.15 (d, J = 8.0 Hz, 1H), 7.12-7.07 |
| | | (m, 2H), 6.87 (dd, J = 5.6, 10.8 Hz, 1H), 6.46 (t, J = 2.4 Hz, 1H), 6.24 (d, J = |
| | | 1.6 Hz, 1H), 6.19 (d, J = 8.0 Hz, 1H), 5.37 (dd, J = 5.2, 12.8 Hz, 1H), 4.52- |
| | | 4.41 (m, 1H), 4.33 (t, J = 7.6 Hz, 2H), 4.11-3.98 (m, 1H), 3.92 (t, J = 6.4 Hz, |
| | | 2H), 3.89-3.81 (m, 1H), 3.70 (s, 3H), 3.36 (s, 3H), 3.20-3.00 (m, 6H), 2.99- |
| | | 2.84 (m, 2H), 2.79-2.70 (m, 1H), 2.69-2.57 (m, 2H), 2.08-1.98 (m, 4H), |
| | | 1.92 (s, 1H), 1.89-1.71 (m, 2H), 1.66-1.38 (m, 1H), 1.10-0.97 (m, 1H) |
| I-491 | 812.3 | 12.08 (s, 1H), 11.09 (s, 1H), 7.43-7.27 (m, 1H), 7.15 (s, 1H), 7.08-7.02 (m, |
| | | 1H), 7.02-6.92 (m, 3H), 6.89 (d, J = 6.0 Hz, 1H), 6.85 (s, 1H), 6.57 (s, 1H), |
| | | 6.19-6.03 (m, 1H), 5.35-5.36 (m, 1H), 4.41-4.24 (m, 2H), 3.95 (d, J = 12.4 |
| | | Hz, 2H), 3.68-3.52 (m, 2H), 3.33 (s, 3H), 3.26-2.98 (m, 6H), 2.96-2.83 (m, |
| | | 3H), 2.83-2.67 (m, 2H), 2.62 (d, J = 17.2 Hz, 1H), 2.38 (s, 1H), 2.27 (s, 1H), |
| | | 2.13-2.05 (m, 3H), 2.05-1.97 (m, 1H), 1.96-1.89 (m, 2H), 1.88-1.76 (m, |
| | | 2H) |
| I-493 | 724.3 | 11.91 (br s, 1H), 10.87 (s, 1H), 7.34 (br t, J = 8.0 Hz, 1H), 7.15-7.01 (m, 3H), |
| | | 6.88-6.76 (m, 1H), 6.47 (br s, 1H), 6.25-6.14 (m, 2H), 6.10 (br d, J = 9.6 Hz, |
| | | 1H), 4.32 (br d, J = 9.6 Hz, 2H), 3.89 (br dd, J = 4.8, 12.0 Hz, 1H), 3.70 (s, 3H), |
| | | 3.64-3.55 (m, 2H), 3.44 (br d, J = 6.4 Hz, 2H), 3.24-3.12 (m, 3H), 3.25- |
| | | 2.94 (m, 5H), 2.84-2.58 (m, 4H), 2.36 (br s, 1H), 2.30-2.17 (m, 2H), 2.07 (br |
| | | d, J = 8.0 Hz, 6H), 1.74 (br dd, J = 8.0, 12.4 Hz, 1H) |
| I-496 | 765.1 | 12.05-11.80 (m, 1H), 10.88-10.73 (m, 1H), 7.27-7.11 (m, 1H), 6.94-6.96 |
| | | (m, 1H), 6.90-6.85 (m, 1H), 6.83 (d, J = 6.4 Hz, 1H), 6.68 (s, 1H), 6.62 (d, J = |
| | | 8.8 Hz, 1H), 6.56-6.43 (m, 2H), 6.17-6.02 (m, 1H), 4.40-4.25 (m, 2H), 3.80- |
| | | 3.75 (m, 1H), 3.75-3.73 (m, 2H), 3.71 (s, 3H), 3.66-3.54 (m, 2H), 3.35 (s, |
| | | 2H), 3.26 (s, 4H), 3.23-2.94 (m, 6H), 2.70-2.59 (m, 1H), 2.54 (s, 2H), 2.46 |
| | | (d, J = 4.0 Hz, 1H), 2.41-2.33 (m, 1H), 2.30-2.21 (m, 1H), 2.07 (d, J = 7.6 |
| | | Hz, 3H), 2.03-1.95 (m, 1H), 1.91 (s, 3H) |
| I-506 | 811.7 | 1.99 (br s, 4 H), 2.39 (br s, 3 H), 2.59-2.71 (m, 2 H), 2.70-2.82 (m, 2 H), 2.84- |
| | | 2.94 (m, 3 H), 3.00-3.11 (m, 3 H), 3.13-3.22 (m, 3 H), 3.28 (br s, 2 H), 3.70 |
| | | (s, 3 H), 3.77 (s, 3 H), 3.78 (br s, 1 H), 4.38 (br s, 2 H), 5.34-5.40 (m, 1 H), |
| | | 6.16 (br s, 1 H), 6.50 (d, J = 2.8 Hz, 1 H), 6.60-6.65 (m, 1 H), 6.89 (br d, J = |
| | | 8.8 Hz, 2 H), 6.95-7.04 (m, 1 H), 6.96 (d, J = 6.4 Hz, 1 H), 6.99-7.03 (m, 1 |
| | | H), 7.09 (s, 1 H), 7.32 (d, J = 8.0 Hz, 1 H), 7.50-7.56 (m, 1 H), 8.12 (br d, J = |
| | | 4.0 Hz, 1 H), 11.00-11.19 (m, 1 H), 12.01 (br s, 1 H) |
| I-515 | 898.5 | 12.14 (s, 1H), 11.10 (s, 1H), 7.40 (d, J = 8.4 Hz, 1H), 7.04 (dd, J = 2.0, 8.8 Hz, |
| | | 1H), 6.97 (d, J = 5.2 Hz, 2H), 6.95-6.82 (m, 3H), 6.53 (s, 1H), 6.12 (s, 1H), |
| | | 5.37 (dd, J = 5.2, 12.4 Hz, 1H), 4.56 (s, 1H), 4.36 (s, 1H), 3.84 (d, J = 4.8 Hz, |
| | | 1H), 3.83-3.72 (m, 2H), 3.65 (s, 1H), 3.56 (s, 3H), 3.29-2.95 (m, 6H), 2.94- |
| | | 2.82 (m, 3H), 2.82-2.66 (m, 3H), 2.66-2.58 (m, 1H), 2.44-2.19 (m, 2H), |
| | | 2.11-1.93 (m, 2H), 1.79 (d, J = 11.2 Hz, 2H), 1.72-1.58 (m, 2H), 1.54-1.44 |
| | | (m, 1H), 1.44-1.33 (m, 2H), 1.28-1.17 (m, 2H), 0.86-0.59 (m, 4H) |
| I-518 | 810.3 | 11.12-10.53 (m, 1H), 7.82-7.75 (m, 2H), 7.72 (J = 2.0, 10.4 Hz, 1H), 7.57- |
| | | 7.47 (m, 2H), 7.46-7.37 (m, 1H), 7.25 (d, J = 13.6 Hz, 1H), 7.17 (s, 1H), 7.16- |
| | | 7.11 (m, 2H), 6.23-6.13 (m, 2H), 4.40-4.35 (m, 3H), 4.30 (s, 1H), 4.18- |
| | | 4.11 (m, 1H), 3.68-3.58 (m, 2H), 3.41 (s, 4H), 3.29 (s, 3H), 3.24 (s, 4H), 3.05 |
| | | (s, 2H), 3.02-2.96 (m, 2H), 2.84-2.73 (m, 1H), 2.56 (d, J = 3.2 Hz, 1H), 2.39- |
| | | 2.24 (m, 4H), 2.00-1.93 (m, 1H) |
| I-519 | 764.4 | 12.21 (br s, 1H), 11.10 (s, 1H), 7.55 (t, J = 8.0 Hz, 1H), 7.38-7.27 (m, 2H), |
| | | 7.08-6.95 (m, 3H), 6.90 (br d, J = 7.6 Hz, 1H), 6.62 (br s, 1H), 6.20-6.12 (m, |
| | | 1H), 5.36 (br dd, J = 5.2, 12.4 Hz, 1H), 4.38-4.30 (m, 2H), 3.70 (s, 3H), 3.62 |
| | | (td, J = 5.6, 11.2 Hz, 2H), 3.25 (br d, J = 11.2 Hz, 2H), 3.21-2.97 (m, 6H), 2.93- |
| | | 2.84 (m, 3H), 2.83-2.75 (m, 1H), 2.73-2.60 (m, 2H), 2.37 (br s, 1H), 2.27 |
| | | (br s, 1H), 2.08 (d, J = 7.6 Hz, 3H), 2.03-1.91 (m, 5H) |
| I-520 | 727.2 | 2.01-2.11 (m, 1 H), 2.25-2.38 (m, 2 H), 2.61-2.76 (m, 2 H), 2.84-2.96 (m, |
| | | 1 H), 3.10 (br s, 2 H), 3.58-3.68 (m, 2 H), 3.78 (br s, 6 H), 4.28-4.41 (m, 2 |
| | | H), 4.63 (br s, 2 H), 5.38-5.48 (m, 1 H), 6.06-6.15 (m, 1 H), 6.22 (br s, 1 H), |
| | | 6.85-6.97 (m, 1 H), 7.05-7.14 (m, 1 H), 7.22 (br d, J = 2.0 Hz, 1 H), 7.28 (br |
| | | s, 2 H), 7.31 (br s, 1 H), 7.40 (br s, 2 H), 7.64-7.73 (m, 1 H), 8.07-8.16 (m, 1 |
| | | H), 11.15 (br s, 1 H), 11.67 (br s, 1 H) |
| I-521 | 739.4 | 11.94 (br s, 1H), 10.79 (s, 1H), 7.17 (br d, J = 8.0 Hz, 1H), 7.04 (br t, J = 8.8 |
| | | Hz, 1H), 6.91-6.80 (m, 1H), 6.69 (s, 1H), 6.63 (br d, J = 8.0 Hz, 1H), 6.53- |
| | | 6.43 (m, 3H), 6.16-6.08 (m, 1H), 6.03 (br d, J = 7.6 Hz, 1H), 4.32 (br d, J = |
| | | 10.0 Hz, 3H), 3.91 (br d, J = 11.2 Hz, 2H), 3.72 (s, 3H), 3.67-3.55 (m, 2H), |
| | | 3.26-2.98 (m, 6H), 2.90-2.65 (m, 4H), 2.62-2.54 (m, 1H), 2.39-2.23 (m, |
| | | 2H), 2.07 (d, J = 7.2 Hz, 4H), 1.94-1.71 (m, 5H) |
| I-523 | 775.3 | 10.86 (s, 1H), 7.79-7.69 (m, 3H), 7.55 (s, 1H), 7.55-7.46 (m, 1H), 7.44 (s, |
| | | 1H), 7.33 (t, J = 8.0 Hz, 1H), 7.14 (d, J = 8.8 Hz, 2H), 7.10-7.03 (m, 2H), 6.26- |
| | | 6.11 (m, 2H), 4.42-4.28 (m, 4H), 3.97 (d, J = 12.8 Hz, 2H), 3.92-3.84 (m, |
| | | 1H), 3.70-3.56 (m, 2H), 3.29 (s, 3H), 3.05 (s, 3H), 3.02-2.95 (m, 3H), 2.93- |
| | | 2.84 (m, 2H), 2.71-2.64 (m, 1H), 2.34 (d, J = 1.6 Hz, 2H), 2.30-2.17 (m, 2H), |
| | | 2.09-1.98 (m, 1H), 1.92-1.78 (m, 4H) |
| I-524 | 858.3 | 11.25-10.88 (m, 1H), 10.45-10.25 (m, 1H), 7.70 (s, 1H), 7.48 (s, 2H), 7.34- |
| | | 7.27 (m, 1H), 7.12-6.97 (m, 5H), 6.93-6.87 (m, 1H), 6.25-6.14 (m, 1H), |
| | | 5.44-5.29 (m, 1H), 4.52-4.40 (m, 1H), 4.37-4.27 (m, 2H), 3.97-3.82 (m, |
| | | 1H), 3.79-3.67 (m, 6H), 3.29-3.23 (m, 3H), 3.00 (br s, 6H), 2.96-2.75 (m, |
| | | 8H), 2.74-2.67 (m, 1H), 2.67-2.54 (m, 2H), 2.00 (br s, 6H), 1.79-1.69 (m, |
| | | 1H), 1.47-1.30 (m, 1H) |
| I-542 | 760.1 | 12.34-12.19 (m, 1H), 10.95-10.86 (m, 1H), 7.72 (d, J = 8.4 Hz, 3H), 7.61 (d, |
| | | J = 8.0 Hz, 2H), 7.46-7.34 (m, 1H), 7.16-7.04 (m, 1H), 6.99-6.88 (m, 2H), |
| | | 6.71-6.63 (m, 1H), 6.22-6.14 (m, 2H), 5.89 (d, J = 7.6 Hz, 1H), 4.52-4.43 |
| | | (m, 1H), 4.40-4.30 (m, 4H), 3.85 (s, 3H), 3.68-3.57 (m, 2H), 3.25-3.02 (m, |
| | | 6H), 3.01-2.95 (m, 2H), 2.84-2.74 (m, 1H), 2.60-2.57 (m, 2H), 2.30-2.24 |
| | | (m, 1H), 2.14-2.02 (m, 2H) |
| I-543 | 790.3 | 12.22-12.06 (m, 1H), 10.87-10.75 (m, 1H), 7.77-7.68 (m, 1H), 7.58-7.53 |
| | | (m, 2H), 7.51-7.34 (m, 1H), 7.12 (d, J = 8.8 Hz, 2H), 7.02-6.94 (m, 1H), 6.92 |
| | | (s, 2H), 6.60-6.53 (m, 1H), 6.49-6.44 (m, 1H), 6.23-6.12 (m, 2H), 5.88 (d, |
| | | J = 7.6 Hz, 1H), 4.41-4.36 (m, 3H), 4.34-4.25 (m, 2H), 3.68-3.57 (m, 2H), |
| | | 3.30-3.07 (m, 6H), 3.04 (s, 4H), 2.96 (t, J = 8.0 Hz, 3H), 2.79-2.71 (m, 1H), |
| | | 2.63-2.54 (m, 2H), 2.52 (s, 2H), 2.36-2.26 (m, 2H), 2.14-2.07 (m, 1H), 1.93- |
| | | 1.81 (m, 1H) |
| I-546 | 811.6 | 12.52-11.84 (m, 1H), 10.64 (d, J = 3.2 Hz, 1H), 7.82 (d, J = 6.4 Hz, 1H), 7.72 |
| | | (dd, J = 2.0, 11.2 Hz, 1H), 7.61 (d, J = 8.0 Hz, 2H), 7.45 (s, 1H), 7.44 (s, 1H), |
| | | 7.35 (d, J = 8.0 Hz, 2H), 7.02 (d, J = 6.0 Hz, 1H), 6.92-6.76 (m, 2H), 6.51 (d, |
| | | J = 7.6 Hz, 1H), 6.26-6.04 (m, 2H), 4.39-4.36 (m, 2H), 4.31 (s, 2H), 3.76 (t, |
| | | J = 5.6 Hz, 2H), 3.67-3.57 (m, 2H), 3.25-3.05 (m, 6H), 3.03-2.93 (m, 3H), |
| | | 2.83 (d, J = 2.8 Hz, 2H), 2.75-2.69 (m, 2H), 2.66 (d, J = 6.0 Hz, 2H), 2.34 (d, |
| | | J = 1.6 Hz, 3H), 1.77 (d, J = 11.6 Hz, 3H), 1.45 (d, J = 9.6 Hz, 2H) |
| I-548 | 739.2 | 12.38-12.03 (m, 1H), 10.99-10.71 (m, 1H), 7.66-7.59 (m, 2H), 7.43 (d, J = |
| | | 8.0 Hz, 2H), 7.10-6.99 (m, 1H), 6.88 (d, J = 3.2 Hz, 1H), 6.67 (d, J = 8.0 Hz, |
| | | 1H), 6.57 (d, J = 14.4 Hz, 1H), 6.22-6.13 (m, 1H), 5.11 (d, J = 6.4 Hz, 1H), |
| | | 4.35 (d, J = 7.6 Hz, 2H), 4.31-4.22 (m, 1H), 3.81 (s, 3H), 3.62 (d, J = 5.6, 11.2 |
| | | Hz, 2H), 3.30-3.29 (m, 1H), 3.24-3.00 (m, 6H), 2.84-2.76 (m, 3H), 2.71- |
| | | 2.66 (m, 1H), 2.59-2.53 (m, 2H), 2.40-2.36 (m, 1H), 2.27 (d, J = 2.8 Hz, 1H), |
| | | 2.17-2.11 (m, 1H), 2.08 (d, J = 6.8 Hz, 3H), 1.96-1.85 (m, 5H) |
| I-551 | 826.3 | 12.03 (s, 1H), 10.88 (d, J = 1.6 Hz, 1H), 7.72 (dd, J = 2.0, 10.8 Hz, 1H), 7.55 |
| | | (d, J = 8.4 Hz, 2H), 7.42 (dd, J = 1.2, 16.0 Hz, 1H), 7.12 (d, J = 8.4 Hz, 2H), |
| | | 7.08 (d, J = 8.8 Hz, 1H), 7.04-6.97 (m, 1H), 6.83 (d, J = 15.2 Hz, 2H), 6.24- |
| | | 6.17 (m, 1H), 5.45 (d, J = 7.6 Hz, 1H), 4.54-4.47 (m, 1H), 4.43 (dd, J = 5.6, |
| | | 12.4 Hz, 1H), 4.36 (q, J = 6.8 Hz, 2H), 3.90 (d, J = 12.4 Hz, 1H), 3.35 (s, 4H), |
| | | 3.25-3.13 (m, 4H), 3.07 (s, 6H), 2.96-2.89 (m, 2H), 2.87-2.73 (m, 2H), 2.61- |
| | | 2.57 (m, 1H), 2.54 (d, J = 3.2 Hz, 1H), 2.14-2.04 (m, 2H), 1.92 (d, J = 4.0 |
| | | Hz, 2H), 1.77 (d, J = 13.2 Hz, 1H), 1.54-1.40 (m, 1H), 1.34-1.16 (m, 1H) |
| I-553 | 669.3 | 7.34 (s, 1H), 7.29-7.22 (m, 1H), 7.08 (dd, J = 1.2, 11.2 Hz, 1H), 7.03 (dd, J = |
| | | 1.2, 7.6 Hz, 1H), 7.00-6.93 (m, 2H), 6.88 (d, J = 6.4 Hz, 1H), 6.46 (d, J = 3.2 |
| | | Hz, 1H), 6.16-6.05 (m, 1H), 4.40-4.26 (m, 2H), 3.89 (dd, J = 4.8, 11.6 Hz, |
| | | 1H), 3.70 (d, J = 1.6 Hz, 3H), 3.65-3.58 (m, 2H), 3.17 (d, J = 4.4 Hz, 6H), 2.96 |
| | | (d, J = 2.0 Hz, 4H), 2.72-2.64 (m, 1H), 2.37 (d, J = 3.2 Hz, 2H), 2.30-2.18 |
| | | (m, 2H), 2.08 (d, J = 7.6 Hz, 3H), 2.05-1.96 (m, 1H) |
| I-555 | 858.3 | 1.33-1.48 (m, 1 H), 1.68-1.80 (m, 1 H), 2.00 (br d, J = 5.2 Hz, 8 H), 2.63- |
| | | 2.74 (m, 2 H), 2.85-2.95 (m, 6 H), 3.00 (br d, J = 2.4 Hz, 8 H), 3.69-3.75 (m, |
| | | 5 H), 3.78 (s, 2 H), 3.81-3.89 (m, 1 H), 3.89-4.01 (m, 1 H), 4.32 (br t, J = 7.2 |
| | | Hz, 2 H), 4.40-4.52 (m, 1 H), 5.37 (dd, J = 12.4, 5.6 Hz, 1 H), 6.20 (t, J = 2.0 |
| | | Hz, 1 H), 6.87-6.92 (m, 1 H), 6.97-7.03 (m, 3 H), 7.05 (s, 1 H), 7.07 (br s, 1 |
| | | H), 7.30 (d, J = 7.6 Hz, 1 H), 7.41 (dd, J = 3.6, 1.6 Hz, 1 H), 7.48 (s, 1 H), 7.70 |
| | | (s, 1 H), 10.35 (br s, 1 H), 11.02-11.15 (m, 1 H) |
| I-562 | 749.4 | 11.97-11.85 (m, 1H), 10.64 (s, 1H), 7.85 (d, J = 6.4 Hz, 1H), 7.48 (s, 1H), 7.27 |
| | | (d, J = 7.6 Hz, 1H), 7.08 (s, 1H), 7.03-6.92 (m, 2H), 6.85 (t, J = 7.2 Hz, 1H), |
| | | 6.60 (d, J = 7.6 Hz, 1H), 6.48 (d, J = 2.4 Hz, 1H), 4.57-4.38 (m, 3H), 3.89- |
| | | 3.82 (m, 1H), 3.81-3.72 (m, 5H), 3.21-3.03 (m, 6H), 2.91-2.80 (m, 5H), |
| | | 2.70 (br s, 1H), 2.54 (br s, 2H), 2.03 (d, J = 8.8 Hz, 3H), 2.01-1.96 (m, 3H), |
| | | 1.96-1.90 (m, 2H), 1.89-1.82 (m, 1H), 1.80-1.73 (m, 1H), 1.61-1.44 (m, |
| | | 1H) |
| I-565 | 718.1 | 12.20 (s, 1H), 11.09 (d, J = 1.2 Hz, 1H), 7.73-7.62 (m, 2H), 7.60-7.52 (m, |
| | | 2H), 7.11-6.95 (m, 2H), 6.87 (d, J = 2.4 Hz, 1H), 6.84-6.75 (m, 2H), 6.22- |
| | | 6.13 (m, 1H), 5.41-5.30 (m, 1H), 4.39-4.25 (m, 4H), 4.02-3.88 (m, 3H), |
| | | 3.69-3.64 (m, 3H), 3.64-3.58 (m, 2H), 3.21-3.01 (m, 6H), 2.93-2.84 (m, |
| | | 1H), 2.66 (d, J = 12 Hz, 1H), 2.38 (s, 1H), 2.30-2.21 (m, 1H), 2.08 (d, J = 7.2 |
| | | Hz, 3H), 2.04-1.97 (m, 1H) |
| I-569 | 805.5 | 11.28-10.79 (m, 1H), 7.36-7.16 (m, 5H), 7.04-6.95 (m, 2H), 6.90 (d, J = |
| | | 6.8 Hz, 1H), 6.28 (s, 1H), 5.36 (dd, J = 5.6, 12.4 Hz, 1H), 4.49-4.30 (m, 2H), |
| | | 3.74-3.61 (m, 5H), 3.25 (d, J = 11.2 Hz, 3H), 3.12-2.86 (m, 9H), 2.72 (dt, J = |
| | | 4.4, 12.8 Hz, 2H), 2.66-2.59 (m, 1H), 2.41-2.36 (m, 1H), 2.27 (d, J = 4.4 |
| | | Hz, 1H), 2.13 (s, 3H), 2.04-1.91 (m, 5H), 1.08-0.95 (m, 6H) |
| I-577 | 815.1 | 12.21-12.09 (m, 1H), 10.78 (s, 1H), 7.77-7.64 (m, 1H), 7.57-7.48 (m, 2H), |
| | | 7.47-7.36 (m, 1H), 7.08 (d, J = 8.8 Hz, 2H), 7.00-6.91 (m, 2H), 6.91-6.78 |
| | | (m, 2H), 6.51-6.53 (m, 1H), 6.30-6.08 (m, 2H), 4.49-4.19 (m, 4H), 3.76- |
| | | 3.70 (m, 4H), 3.68-3.52 (m, 2H), 3.24 (s, 4H), 3.23-2.99 (m, 6H), 2.98-2.94 |
| | | (m, 1H), 2.69-2.55 (m, 2H), 2.33 (d, J = 1.6 Hz, 1H), 2.27 (s, 1H), 2.22-2.09 |
| | | (m, 1H), 2.05-1.96 (m, 1H), 1.89 (s, 4H), 1.34-1.18 (m, 2H) |
| I-580 | 771.3 | 11.92-11.88 (s, 1H), 10.90-10.80 (s, 1H), 7.27-7.24 (d, J = 7.6 Hz, 1H), |
| | | 7.07-7.05(s, 1H), 6.99-6.92 (m, 2H), 6.69-6.62 (d, J = 8.4 Hz, 1H), 6.59- |
| | | 6.55 (d, J = 14.4 Hz, 1H), 6.48-6.45 (s, 1H), 5.12-5.09 (d, J = 7.2 Hz, 1H), |
| | | 4.49-4.43 (m, 1H), 4.29-4.24 (m, 1H), 3.87-3.83 (m, 1H), 3.82-3.80 (s, |
| | | 3H), 3.75-3.74 (s, 3H), 3.21-3.13 (m, 3H), 3.12-3.06 (m, 3H), 3.005-2.98 |
| | | (m, 2H), 2.83-2.77 (m, 3H), 2.74-2.63 (m, 3H), 2.58-2.55 (m, 2H), 2.17- |
| | | 2.12 (m, 1H), 2.04-2.01 (m, 3H), 1.98-1.94 (m, 2H), 1.93-1.91 (m, 3H), |
| | | 1.91-1.88 (m, 2H), 1.85-1.70 (m, 2H). |
| I-584 | 831.4 | 1.9-2.0 (m, 1 H), 2.2-2.2 (m, 1 H), 2.2-2.3 (m, 1 H), 2.3-2.4 (m, 3 H), 2.6- |
| | | 2.7 (m, 1 H), 2.6-2.7 (m, 3 H), 2.7-2.7 (m, 2 H), 2.8-2.9 (m, 2 H), 3.1- |
| | | 3.1 (m, 4 H), 3.3 (br s, 6 H), 3.6 (br t, J = 5.6 Hz, 1 H), 3.6-3.7 (m, 1 H), 3.7- |
| | | 3.7 (m, 3 H), 4.2-4.3 (m, 1 H), 4.4-4.4 (m, 2 H), 4.6-4.6 (m, 2 H), 5.4-5.5 |
| | | (m, 1 H), 6.1-6.2 (m, 1 H), 6.5-6.5 (m, 1 H), 6.6-6.6 (m, 2 H), 6.7-6.7 (m, |
| | | 2 H), 6.8-6.9 (m, 1 H), 7.0-7.1 (m, 2 H), 7.1-7.2 (m, 1 H), 7.6-7.7 (m, 1 |
| | | H), 8.1-8.1 (m, 1 H), 10.8-10.9 (m, 1 H), 11.9-12.0 (m, 1 H) |
| I-586 | 790.5 | 12.04 (s, 1H), 11.18-10.97 (m, 1H), 7.31-7.20 (m, 3H), 7.05-6.87 (m, 4H), |
| | | 6.39 (s, 1H), 5.37 (dd, J = 5.2, 12.8 Hz, 1H), 4.54-4.43 (m, 1H), 4.06-3.88 |
| | | (m, 1H), 3.70 (s, 3H), 3.25 (d, J = 11.2 Hz, 3H), 3.17-3.03 (m, 6H), 2.94-2.84 |
| | | (m, 4H), 2.78-2.57 (m, 5H), 2.17 (s, 3H), 2.01-1.75 (m, 8H), 1.60-1.41 (m, |
| | | 1H), 1.08-0.94 (m, 6H) |
| I-587 | 761.3 | 12.12 (d, J = 7.6 Hz, 1H), 10.80 (s, 1H), 7.75-7.68 (m, 1H), 7.54-7.47 (m, |
| | | 2H), 7.45-7.37 (m, 1H), 7.20 (t, J = 8.8 Hz, 1H), 6.99-6.90 (m, 1H), 6.84 (s, |
| | | 1H), 6.64 (d, J = 8.4 Hz, 2H), 6.56-6.46 (m, 2H), 6.23-6.17 (m, 1H), 6.17- |
| | | 6.11 (m, 2H), 4.40-4.34 (m, 4H), 4.33-4.25 (m, 3H), 4.11-4.00 (m, 1H), |
| | | 3.84 (t, J = 6.8 Hz, 2H), 3.67-3.57 (m, 2H), 3.26-3.03 (m, 6H), 3.01-2.95 |
| | | (m, 2H), 2.80-2.70 (m, 1H), 2.68-2.60 (m, 1H), 2.35-2.27 (m, 2H), 2.14- |
| | | 2.06 (m, 1H), 1.94-1.82 (m, 1H) |
| I-589 | 830.2 | 12.18-12.06 (m, 1H), 10.81-10.70 (m, 1H), 7.74-7.69 (m, 1H), 7.53-7.37 |
| | | (m, 3H), 7.07 (d, J = 8.8 Hz, 2H), 6.98-6.91 (m, 1H), 6.85 (s, 1H), 6.50 (d, J = |
| | | 15.6 Hz, 1H), 6.42-6.36 (m, 2H), 6.22-6.12 (m, 2H), 5.53 (d, J = 7.2 Hz, 1H), |
| | | 4.46-4.26 (m, 5H), 4.23-4.16 (m, 1H), 3.65 (t, J = 5.6 Hz, 1H), 3.57 (s, 5H), |
| | | 3.23 (s, 6H), 3.10-3.05 (m, 2H), 3.00-2.96 (m, 2H), 2.75-2.67 (m, 1H), 2.33 |
| | | (s, 2H), 2.26 (s, 1H), 2.12-2.06 (m, 1H), 1.92-1.77 (m, 6H) |
| I-590 | 730.1 | 12.26 (s, 1H), 10.86 (s, 1H), 7.76-7.67 (m, 3H), 7.59 (d, J = 8.0 Hz, 2H), 7.46- |
| | | 7.36 (m, 1H), 7.31 (t, J = 8.4 Hz, 1H), 7.14-7.03 (m, 1H), 6.89 (s, 1H), 6.72 |
| | | (d, J = 7.6 Hz, 1H), 6.64-6.51 (m, 2H), 6.24-6.13 (m, 2H), 4.47 (dd, J = 4.8, |
| | | 7.6, 12.0 Hz, 1H), 4.41-4.30 (m, 4H), 3.68-3.56 (m, 2H), 3.28-3.03 (m, |
| | | 6H), 3.00-2.94 (m, 2H), 2.80-2.71 (m, 1H), 2.62-2.56 (m, 1H), 2.36-2.25 |
| | | (m, 2H), 2.16-2.07 (m, 1H), 1.92 (dd, J = 4.4, 12.4 Hz, 1H) |
| I-592 | 775.3 | 12.12 (br d, J = 8.0 Hz, 1H), 10.42 (s, 1H), 7.75-7.68 (m, 1H), 7.53 (br d, J = |
| | | 8.4 Hz, 2H), 7.45-7.35 (m, 2H), 7.24-7.19 (m, 1H), 7.15 (br d, J = 8.4 Hz, |
| | | 1H), 7.11 (br d, J = 8.4 Hz, 2H), 7.00-6.93 (m, 1H), 6.87 (br s, 1H), 6.26- |
| | | 6.10 (m, 2H), 4.40-4.28 (m, 4H), 3.92 (br d, J = 12.0 Hz, 2H), 3.80 (t, J = 6.4 |
| | | Hz, 2H), 3.67-3.55 (m, 2H), 3.22-3.05 (m, 5H), 3.04-2.93 (m, 4H), 2.92- |
| | | 2.81 (m, 2H), 2.73-2.67 (m, 2H), 2.35-2.24 (m, 2H), 1.91-1.80 (m, 4H) |
| I-594 | 774.1 | 12.41-12.08 (m, 1H), 11.02-10.77 (m, 1H), 9.04-8.99 (m, 1H), 8.45-8.41 |
| | | (m, 1H), 8.08 (s, 1H), 7.92-7.89 (m, 1H), 7.87-7.84 (m, 1H), 7.81-7.78 (m, |
| | | 2H), 7.75-7.70 (m, 3H), 7.44-7.37 (m, 1H), 7.16-7.09 (m, 1H), 6.91 (br s, |
| | | 1H), 6.19 (br d, J = 13.2 Hz, 2H), 4.85-4.77 (m, 1H), 4.38 (br d, J = 6.4 Hz, |
| | | 2H), 4.36-4.30 (m, 2H), 3.67-3.58 (m, 2H), 3.24-3.16 (m, 3H), 3.11-3.01 |
| | | (m, 3H), 3.01-2.97 (m, 2H), 2.86-2.77 (m, 1H), 2.59-2.54 (m, 1H), 2.35- |
| | | 2.27 (m, 2H), 2.18-2.08 (m, 1H), 2.03-1.97 (m, 1H) |
| I-598 | 833.3 | 12.37-11.98 (m, 1H), 11.06-10.63 (m, 1H), 8.77 (d, J = 8.4 Hz, 1H), 7.96 (d, |
| | | J = 2.0 Hz, 1H), 7.88-7.81 (m, 1H), 7.75-7.70 (m, 1H), 7.64 (d, J = 8.0 Hz, |
| | | 2H), 7.45 (d, J = 8.0 Hz, 3H), 7.30 (d, J = 8.4 Hz, 1H), 7.09-7.00 (m, 1H), 6.88 |
| | | (s, 1H), 6.30-6.03 (m, 2H), 4.86-4.71 (m, 1H), 4.42-4.29 (m, 4H), 3.69- |
| | | 3.51 (m, 4H), 3.21-3.04 (m, 6H), 3.02-2.97 (m, 2H), 2.88 (t, J = 10.4 Hz, |
| | | 2H), 2.84-2.75 (m, 2H), 2.57 (d, J = 3.2 Hz, 1H), 2.36-2.26 (m, 2H), 2.15- |
| | | 2.06 (m, 1H), 2.03-1.85 (m, 5H) |
| I-603 | 823.0 | 12.24 (s, 1H), 10.88 (s, 1H), 7.63-7.55 (m, 1H), 7.51-7.41 (m, 2H), 7.07- |
| | | 6.94 (m, 1H), 6.68 (d, J = 8.0 Hz, 1H), 6.62-6.51 (m, 2H), 6.20-6.10 (m, 1H), |
| | | 5.19-5.07 (m, 1H), 4.38-4.32 (m, 2H), 4.31-4.23 (m, 1H), 3.84 (s, 3H), 3.65- |
| | | 3.58 (m, 2H), 3.32 (s, 1H), 3.23-2.99 (m, 6H), 2.87-2.76 (m, 4H), 2.70- |
| | | 2.55 (m, 2H), 2.39-2.27 (m, 2H), 2.18-2.11 (m, 1H), 2.08 (d, J = 8.4 Hz, 3H), |
| | | 1.99-1.92 (m, 3H), 1.91-1.82 (m, 2H) |
| I-606 | 777.4 | 11.18 (s, 1H), 7.39-7.22 (m, 4H), 7.22-7.16 (m, 1H), 7.07-6.95 (m, 2H), |
| | | 6.94-6.86 (m, 1H), 6.34-6.25 (m, 1H), 5.37 (dd, J = 5.6, 12.4 Hz, 1H), 4.40- |
| | | 4.31 (m, 2H), 3.71 (s, 3H), 3.65-3.58 (m, 2H), 3.26 (br d, J = 11.2 Hz, 3H), |
| | | 3.19-2.98 (m, 6H), 2.92-2.87 (m, 2H), 2.80-2.61 (m, 3H), 2.43-2.26 (m, |
| | | 3H), 2.16-2.06 (m, 6H), 1.97 (br d, J = 2.4 Hz, 4H) |
| I-609 | 777.2 | 11.06 (s, 1H), 8.45 (s, 1H), 7.37-7.24 (m, 4H), 7.22-7.17 (m, 1H), 7.14 (s, |
| | | 1H), 7.08 (br d, J = 8.0 Hz, 1H), 6.70 (d, J = 7.2 Hz, 1H), 6.34-6.22 (m, 1H), |
| | | 5.70 (dd, J = 5.2, 11.6 Hz, 1H), 4.36 (br s, 2H), 3.76 (s, 3H), 3.68-3.57 (m, |
| | | 2H), 3.54-3.44 (m, 2H), 3.28-2.95 (m, 6H), 2.92-2.81 (m, 4H), 2.76-2.65 |
| | | (m, 5H), 2.42-2.37 (m, 1H), 2.32-2.19 (m, 2H), 2.09 (d, J = 6.8 Hz, 3H), 2.06- |
| | | 2.01 (m, 3H) |
| I-618 | 802.2 | 12.23 (s, 1H), 11.10 (s, 1H), 7.65-7.54 (m, 3H), 7.07-6.93 (m, 2H), 6.88- |
| | | 6.77 (m, 2H), 6.58-6.51 (m, 1H), 6.20-6.09 (m, 1H), 5.43-5.28 (m, 1H), |
| | | 4.38-4.28 (m, 4H), 4.08-4.00 (m, 1H), 3.96-3.88 (m, 2H), 3.66 (s, 3H), 3.64- |
| | | 3.57 (m, 2H), 3.21-3.10 (m, 3H), 3.03 (d, J = 5.6 Hz, 3H), 2.92-2.84 (m, |
| | | 1H), 2.79-2.70 (m, 1H), 2.69-2.63 (m, 2H), 2.31-2.23 (m, 1H), 2.11-2.05 |
| | | (m, 3H), 2.04-1.98 (m, 1H) |
| I-619 | 803.3 | 12.17 (d, J = 6.8 Hz, 1H), 10.80 (s, 1H), 7.74-7.68 (m, 1H), 7.58 (d, J = 7.6 |
| | | Hz, 2H), 7.45-7.34 (m, 3H), 7.09-6.97 (m, 2H), 6.85 (s, 1H), 6.52-6.44 (m, |
| | | 2H), 6.22-6.11 (m, 3H), 4.39-4.28 (m, 5H), 3.64 (s, 1H), 3.59-3.56 (m, 1H), |
| | | 3.40 (s, 2H), 3.22-3.02 (m, 6H), 2.99-2.91 (m, 4H), 2.78-2.71 (m, 1H), 2.61 |
| | | (s, 1H), 2.56 (s, 1H), 2.33 (s, 1H), 2.27 (s, 1H), 2.13-2.01 (m, 3H), 1.94-1.85 |
| | | (m, 1H), 1.78 (d, J = 11.6 Hz, 2H), 1.73-1.62 (m, 2H) |
| I-621 | 803.2 | 12.21-12.12 (m, 1H), 10.76 (s, 1H), 7.78-7.66 (m, 1H), 7.59 (d, J = 7.6 Hz, |
| | | 2H), 7.45-7.36 (m, 1H), 7.32 (d, J = 8.0 Hz, 2H), 7.10-6.95 (m, 1H), 6.87 (s, |
| | | 1H), 6.82-6.85 (m, 1H), 6.49-6.52 (m, 1H), 6.40-6.43 (m, 1H), 6.27-6.05 |
| | | (m, 2H), 5.77 (d, J = 7.6 Hz, 1H), 4.42-4.33 (m, 3H), 4.32-4.17 (m, 2H), 3.75- |
| | | 3.50 (m, 2H), 3.30-3.10 (m, 6H), 3.07 (d, J = 3.2 Hz, 2H), 3.01-2.92 (m, |
| | | 2H), 2.79-2.67 (m, 1H), 2.62 (d, J = 6.4 Hz, 2H), 2.60-2.53 (m, 2H), 2.33 (s, |
| | | 2H), 2.27 (d, J = 1.2 Hz, 1H), 2.13-2.02 (m, 1H), 1.84-1.86 (m, 1H), 1.74- |
| | | 1.56 (m, 3H), 1.47-1.27 (m, 2H). |
| I-622 | 802.1 | 12.32-11.82 (m, 1H), 10.96-10.49 (m, 1H), 7.76-7.68 (m, 1H), 7.49 (br d, |
| | | J = 8.4 Hz, 2H), 7.45-7.35 (m, 1H), 6.96-6.87 (m, 1H), 6.83 (d, J = 2.8 Hz, |
| | | 1H), 6.57 (d, J = 8.4 Hz, 2H), 6.51 (br d, J = 16.0 Hz, 1H), 6.42 (br d, J = 5.6 |
| | | Hz, 1H), 6.23-6.16 (m, 1H), 6.12 (br s, 1H), 5.58 (d, J = 7.6 Hz, 1H), 4.39- |
| | | 4.33 (m, 3H), 4.29 (br s, 1H), 4.21 (ddd, J = 4.8, 7.2, 11.2 Hz, 1H), 4.03 (s, 4H), |
| | | 3.93 (s, 4H), 3.66-3.56 (m, 2H), 3.25-3.02 (m, 6H), 3.00-2.94 (m, 2H), 2.76- |
| | | 2.67 (m, 1H), 2.62-2.56 (m, 1H), 2.54 (s, 1H), 2.33 (br s, 1H), 2.26 (br d, J = |
| | | 2.4 Hz, 1H), 2.14-2.04 (m, 1H), 1.91-1.77 (m, 1H) |
| I-629 | 775.2 | 12.28-12.08 (m, 1H), 10.37 (s, 1H), 7.74-7.69 (m, 1H), 7.62 (d, J = 8.0 Hz, |
| | | 2H), 7.46-7.36 (m, 3H), 7.23-7.17 (m, 1H), 7.12-7.00 (m, 3H), 6.88 (d, J = |
| | | 2.8 Hz, 1H), 6.22-6.13 (m, 2H), 4.39-4.30 (m, 4H), 3.76 (t, J = 6.8 Hz, 2H), |
| | | 3.67-3.57 (m, 2H), 3.49 (d, J = 12.0 Hz, 2H), 3.21-3.04 (m, 5H), 3.04-2.94 |
| | | (m, 3H), 2.83 (t, J = 10.4 Hz, 2H), 2.79-2.73 (m, 1H), 2.70 (t, J = 6.8 Hz, 2H), |
| | | 2.33 (s, 2H), 1.93 (s, 2H), 1.90 (s, 2H) |
| I-634 | 824.2 | 12.19-12.12 (s, 1H), 10.88-10.81 (s, 1H), 7.49-7.45 (m, 1H), 7.14-7.10 |
| | | (d, J = 8.8 Hz, 1H), 7.03-7.00 (s, 1H), 6.97-6.89 (m, 1H), 6.69-6.65 (d, J = |
| | | 8.0 Hz, 1H), 6.62-6.54 (m, 2H), 6.15-6.10 (m, 1H), 5.16-5.12 (d, J = 6.8 |
| | | Hz, 1H), 4.34-4.30 (m, 2H), 4.30-4.24 (s, 1H), 3.83-3.80 (s, 3H), 3.64- |
| | | 3.58 (m, 2H), 3.43-4.39 (m, 4H), 3.12-3.09 (m, 6H), 2.85-2.77 (m, 1H), |
| | | 2.72-2.63 (m, 1H), 2.57-2.55 (m, 1H), 2.39-2.36 (s, 2H), 2.34-2.31 (m, |
| | | 1H), 2.29-2.25 (s, 1H), 2.19-2.10 (m, 2H), 2.09-2.06 (m, 3H), 1.98-1.91 |
| | | (m, 1H) |
| I-636 | 820.1 | 12.01 (d, J = 8.4 Hz, 1H), 10.80 (s, 1H), 8.14-8.06 (m, 1H), 7.72-7.64 (m, |
| | | 1H), 7.30 (d, J = 7.6 Hz, 1H), 7.14-7.07 (m, 1H), 7.06-7.02 (m, 1H), 7.02- |
| | | 6.95 (m, 3H), 6.95-6.88 (m, 1H), 6.48 (s, 1H), 6.17-6.08 (m, 1H), 4.67-4.57 |
| | | (m, 2H), 4.39-4.29 (m, 2H), 3.80 (dd, J = 4.8, 11.6 Hz, 1H), 3.75-3.70 (m, |
| | | 3H), 3.67-3.56 (m, 4H), 3.21-2.98 (m, 12H), 2.70-2.65 (m, 1H), 2.61 (d, J = |
| | | 4.0 Hz, 5H), 2.33 (dd, J = 2.0, 3.6 Hz, 1H), 2.30-2.23 (m, 1H), 2.23-2.12 |
| | | (m, 1H), 2.00 (qd, J = 4.4, 13.2 Hz, 1H) |
| I-641 | 761.2 | 10.89 (s, 1H), 7.45-7.22 (m, 5H), 7.19 (s, 1H), 7.12-7.01 (m, 2H), 6.28 (s, |
| | | 1H), 4.40-4.28 (m, 6H), 3.66-3.57 (m, 2H), 3.37 (d, J = 10.0 Hz, 2H), 3.22- |
| | | 2.95 (m, 6H), 2.89 (s, 2H), 2.79 (d, J = 6.0 Hz, 1H), 2.69-2.61 (m, 2H), 2.40- |
| | | 2.25 (m, 3H), 2.23-2.16 (m, 1H), 2.13 (d, J = 5.6 Hz, 3H), 2.08 (d, J = 4.0 Hz, |
| | | 3H), 2.01 (s, 4H) |
| I-644 | 832.3 | 12.19-12.04 (m, 1H), 11.16-11.04 (m, 1H), 7.62-7.56 (m, 1H), 7.50 (br d, |
| | | J = 8.0 Hz, 1H), 7.45 (s, 1H), 7.06-6.95 (m, 3H), 6.90 (br d, J = 7.2 Hz, 1H), |
| | | 6.53 (br s, 1H), 5.42 (s, 1H), 4.46 (br d, J = 12.4 Hz, 1H), 3.90-3.81 (m, 1H), |
| | | 3.70 (s, 3H), 3.26 (br d, J = 12.0 Hz, 3H), 3.19-2.99 (m, 7H), 2.95-2.83 (m, |
| | | 4H), 2.74-2.64 (m, 2H), 2.62-2.55 (m, 1H), 2.06-2.01 (m, 4H), 1.99 (br s, |
| | | 3H), 1.95-1.71 (m, 4H), 1.66-1.40 (m, 1H) |
| I-645 | 780.0 | 12.27-12.10 (m, 1H), 11.21-11.02 (m, 1H), 7.57 (s, 1H), 7.49-7.38 (m, 2H), |
| | | 7.07-6.85 (m, 4H), 6.51-6.42 (m, 1H), 6.22-6.07 (m, 1H), 5.41-5.32 (m, |
| | | 1H), 4.40-4.27 (m, 2H), 3.70 (s, 3H), 3.61 (td, J = 5.6, 10.8 Hz, 2H), 3.25 (br |
| | | d, J = 10.4 Hz, 2H), 3.21-2.96 (m, 6H), 2.91-2.85 (m, 2H), 2.82-2.75 (m, |
| | | 1H), 2.73-2.59 (m, 2H), 2.54 (br s, 1H), 2.37 (br d, J = 2.8 Hz, 1H), 2.26 (br |
| | | d, J = 2.8 Hz, 1H), 2.08 (d, J = 5.6 Hz, 3H), 2.04-1.89 (m, 5H) |
| I-646 | 782.2 | 12.23-11.83 (m, 1H), 11.25-10.91 (m, 1H), 7.57 (s, 1H), 7.47-7.40 (m, 2H), |
| | | 7.04-6.95 (m, 3H), 6.90 (br d, J = 7.2 Hz, 1H), 6.49-6.42 (m, 1H), 5.36 (dd, |
| | | J = 5.6, 12.4 Hz, 1H), 4.52-4.42 (m, 1H), 3.86 (br d, J = 13.6 Hz, 1H), 3.70 (s, |
| | | 3H), 3.25 (br d, J = 11.6 Hz, 4H), 3.21-2.97 (m, 8H), 2.90-2.85 (m, 2H), 2.83- |
| | | 2.77 (m, 1H), 2.71 (br dd, J = 4.4, 12.8 Hz, 1H), 2.65-2.59 (m, 1H), 2.54 (s, |
| | | 1H), 2.03 (d, J = 7.6 Hz, 3H), 2.00-1.93 (m, 5H), 1.88-1.69 (m, 2H), 1.63- |
| | | 1.42 (m, 1H) |
| I-647 | 816.4 | 12.19-12.07 (m, 1H), 10.90 (br s, 1H), 7.62-7.58 (m, 1H), 7.55-7.51 (m, |
| | | 1H), 7.48 (s, 1H), 7.41 (d, J = 7.6 Hz, 1H), 7.11-7.02 (m, 3H), 6.54 (d, J = 2.4 |
| | | Hz, 1H), 4.51-4.44 (m, 1H), 4.38-4.34 (m, 1H), 4.33 (s, 3H), 3.87 (br d, J = |
| | | 12.8 Hz, 1H), 3.40 (br s, 4H), 3.26-2.97 (m, 10H), 2.91 (br s, 3H), 2.74-2.56 |
| | | (m, 4H), 2.40-2.14 (m, 3H), 1.98-1.73 (m, 4H), 1.62-1.43 (m, 1H) |
| I-649 | 830.1 | 12.35-12.05 (m, 1H), 11.24-10.96 (m, 1H), 7.61-7.58 (m, 1H), 7.52-7.49 |
| | | (m, 1H), 7.46-7.44 (s, 1H), 7.04-7.01 (s, 1H), 6.99-6.98 (m, 1H), 6.97- |
| | | 6.94 (m, 1H), 6.91-6.87 (m, 1H), 6.55-6.52 (d, J = 3.2 Hz, 1H), 6.17-6.11 |
| | | (m, 1H), 5.39-5.33 (m, 1H), 4.36-4.31 (m, 2H), 3.71-3.68 (s, 3H), 3.63- |
| | | 3.58 (m, 2H), 3.27-3.24 (m, 2H), 3.20-3.12 (m, 3H), 3.09-3.00 (m, 3H), |
| | | 2.92-2.88 (m, 2H), 2.87-2.85 (s, 1H), 2.76-2.69 (m, 1H), 2.65-2.62 (m, |
| | | 1H), 2.62-2.59 (m, 1H), 2.40-2.36 (m, 2H), 2.29-2.24 (s, 1H), 2.09-2.06 |
| | | (m, 3H), 2.02-1.99 (m, 2H), 1.98-1.93 (m, 2H) |
| I-658 | 747.6 | 12.28 (s, 1H), 11.10 (s, 1H), 8.88-8.83 (m, 1H), 8.05 (br dd, J = 2.4, 8.0 Hz, |
| | | 1H), 7.50 (br d, J = 8.0 Hz, 1H), 7.20-7.08 (m, 1H), 7.05-6.97 (m, 2H), 6.90 |
| | | (br s, 2H), 6.21 (br s, 1H), 5.37 (br dd, J = 5.2, 12.8 Hz, 1H), 4.37 (br d, J = 7.2 |
| | | Hz, 2H), 3.71 (s, 3H), 3.63 (td, J = 5.6, 11.6 Hz, 2H), 3.27 (br d, J = 10.8 Hz, |
| | | 6H), 3.14-3.00 (m, 3H), 2.94-2.87 (m, 3H), 2.78-2.68 (m, 1H), 2.64 (br d, |
| | | J = 18.0 Hz, 1H), 2.40 (br s, 1H), 2.29 (br d, J = 3.2 Hz, 1H), 2.10 (d, J = 4.8 |
| | | Hz, 4H), 2.07-2.00 (m, 4H) |
| I-660 | 837.1 | 12.27-12.12 (m, 1H), 10.91-10.77 (m, 1H), 7.77-7.67 (m, 1H), 7.64 (d, J = |
| | | 7.6 Hz, 2H), 7.49-7.34 (m, 3H), 7.10-6.98 (m, 1H), 6.88 (s, 1H), 6.50-6.42 |
| | | (m, 1H), 6.24-6.12 (m, 2H), 5.68 (d, J = 8.4 Hz, 1H), 4.44-4.35 (m, 4H), 4.32 |
| | | (s, 1H), 3.79-3.72 (m, 3H), 3.67-3.56 (m, 2H), 3.27-3.13 (m, 6H), 3.12 (d, |
| | | J = 9.2 Hz, 3H), 3.04-2.91 (m, 3H), 2.80-2.67 (m, 2H), 2.56 (s, 1H), 2.36- |
| | | 2.26 (m, 2H), 2.08 (d, J = 7.2 Hz, 2H), 1.91-1.78 (m, 4H) |
| I-661 | 807.0 | 12.18 (br d, J = 8.0 Hz, 1H), 10.81 (s, 1H), 7.75-7.68 (m, 1H), 7.62 (br d, J = |
| | | 8.0 Hz, 2H), 7.45-7.35 (m, 3H), 7.08-6.98 (m, 1H), 6.93 (br dd, J = 8.4, 13.2 |
| | | Hz, 1H), 6.87 (br d, J = 2.4 Hz, 1H), 6.77 (dd, J = 8.4, 14.4 Hz, 1H), 6.22-6.12 |
| | | (m, 2H), 4.43-4.27 (m, 6H), 3.69-3.55 (m, 4H), 3.30 (br s, 3H), 3.25-3.03 |
| | | (m, 6H), 3.01-2.92 (m, 3H), 2.82-2.65 (m, 4H), 2.57 (br dd, J = 3.6, 17.6 Hz, |
| | | 1H), 2.38-2.21 (m, 2H), 2.10-1.97 (m, 2H), 1.94-1.83 (m, 4H) |
| I-663 | 777.3 | 10.89 (s, 1H), 7.40 (d, J = 7.2 Hz, 1H), 7.36-7.22 (m, 3H), 7.14 (s, 1H), 7.11- |
| | | 7.00 (m, 3H), 6.26 (s, 1H), 4.42-4.27 (m, 6H), 3.76 (s, 3H), 3.66-3.56 (m, |
| | | 2H), 3.39 (d, J = 10.8 Hz, 2H), 3.24-2.96 (m, 6H), 2.95-2.81 (m, 3H), 2.70- |
| | | 2.61 (m, 2H), 2.41-2.27 (m, 3H), 2.22-2.14 (m, 1H), 2.11-1.99 (m, 7H) |
| I-666 | 760.5 | 12.13 (s, 1H), 11.11-11.02 (m, 1H), 7.29-7.18 (m, 3H), 6.96 (d, J = 8.8 Hz, |
| | | 1H), 6.90 (d, J = 2.0 Hz, 1H), 6.80 (d, J = 6.0 Hz, 1H), 6.70 (dd, J = 2.0, 8.4 Hz, |
| | | 1H), 6.38 (s, 1H), 6.19-6.11 (m, 1H), 5.30 (dd, J = 5.2, 12.8 Hz, 1H), 4.37- |
| | | 4.30 (m, 2H), 3.75 (d, J = 12.0 Hz, 2H), 3.60 (td, J = 5.6, 10.8 Hz, 2H), 3.20- |
| | | 2.97 (m, 7H), 2.94-2.86 (m, 1H), 2.81-2.74 (m, 2H), 2.72-2.63 (m, 3H), |
| | | 2.60 (d, J = 2.4 Hz, 1H), 2.39-2.30 (m, 2H), 2.29-2.22 (m, 1H), 2.17-2.14 |
| | | (m, 3H), 2.07 (d, J = 5.2 Hz, 3H), 2.03-1.90 (m, 4H), 1.89-1.85 (m, 1H) |
| I-667 | 762.3 | 11.91 (s, 1H), 10.89 (s, 1H), 7.48-7.24 (m, 2H), 7.13-7.01 (m, 4H), 6.95 (dd, |
| | | J = 6.0, 9.2 Hz, 1H), 6.48 (s, 1H), 4.47 (t, J = 11.2 Hz, 1H), 4.40-4.28 (m, 4H), |
| | | 3.86 (d, J = 12.0 Hz, 1H), 3.76 (s, 3H), 3.38 (d, J = 11.2 Hz, 2H), 3.22-2.97 |
| | | (m, 7H), 2.93-2.78 (m, 3H), 2.76-2.53 (m, 4H), 2.34 (d, J = 9.6 Hz, 1H), 2.18 |
| | | (dd, J = 5.2, 12.8 Hz, 1H), 2.03 (d, J = 7.2 Hz, 7H), 1.95-1.72 (m, 3H), 1.65- |
| | | 1.40 (m, 1H) |
| I-674 | 806.6 | 12.20 (s, 1H), 11.18-11.02 (m, 1H), 7.40 (d, J = 7.6 Hz, 1H), 7.12 (d, J = 11.2 |
| | | Hz, 1H), 7.04-6.98 (m, 2H), 6.93-6.85 (m, 2H), 6.44 (s, 1H), 6.16 (s, 1H), |
| | | 5.41-5.34 (m, 1H), 4.42-4.35 (m, 2H), 3.70 (s, 3H), 3.68 (d, J = 4.8 Hz, 2H), |
| | | 3.28 (d, J = 10.4 Hz, 2H), 3.21-3.02 (m, 6H), 2.96 (s, 2H), 2.94 (d, J = 11.2 |
| | | Hz, 2H), 2.90-2.86 (m, 1H), 2.77-2.69 (m, 1H), 2.64 (d, J = 17.2 Hz, 1H), |
| | | 2.39-2.25 (m, 2H), 2.16 (s, 3H), 2.08-1.99 (m, 3H), 1.96-1.90 (m, 2H), 1.07- |
| | | 1.00 (m, 6H) |
| I-804 | 752.1 | 11.99 (s, 1H), 10.81 (s, 1H), 7.28 (t, J = 8.0 Hz, 1H), 7.16 (d, J = 8.4 Hz, 1H), |
| | | 7.09-6.97 (m, 2H), 6.91-6.80 (m, 1H), 6.65 (s, 1H), 6.59 (d, J = 8.4 Hz, 1H), |
| | | 6.49 (s, 1H), 6.15-6.07 (m, 1H), 4.37-4.26 (m, 2H), 3.92-3.84 (m, 1H), 3.80 |
| | | (d, J = 12.0 Hz, 2H), 3.71 (s, 3H), 3.61 (d, J = 5.6 Hz, 2H), 3.22-2.94 (m, 6H), |
| | | 2.79-2.70 (m, 2H), 2.68 (d, J = 6.4 Hz, 2H), 2.47-2.32 (m, 3H), 2.30-2.15 |
| | | (m, 2H), 2.11-2.05 (m, 3H), 2.02 (d, J = 4.4 Hz, 1H), 1.91-1.80 (m, 2H), 1.61- |
| | | 1.51 (m, 2H), 1.51-1.41 (m, 1H), 1.38-1.26 (m, 2H). |
| I-806 | 830.3 | 12.19-12.10 (m, 1H), 11.16-11.05 (m, 1H), 7.48-7.42 (m, 1H), 7.16-7.12 |
| | | (m, 1H), 7.05-6.97 (m, 4H), 6.97-6.88 (m, 1H), 6.57-6.52 (m, 1H), 6.16- |
| | | 6.10 (m, 1H), 5.39 (br dd, J = 5.2, 12.8 Hz, 1H), 4.35-4.30 (m, 2H), 4.01-3.91 |
| | | (m, 2H), 3.67-3.57 (m, 5H), 3.53-3.46 (m, 1H), 3.20-3.02 (m, 6H), 3.00- |
| | | 2.87 (m, 3H), 2.77-2.69 (m, 1H), 2.60 (br s, 1H), 2.38 (br s, 1H), 2.27 (br d, J = |
| | | 1.6 Hz, 1H), 2.07 (d, J = 8.8 Hz, 3H), 2.04-1.95 (m, 3H), 1.93-1.84 (m, 2H) |
| I-675 | 811.1 | 12.09-12.01 (m, 1H), 10.58-10.50 (m, 1H), 7.74-7.69 (m, 1H), 7.53-7.46 |
| | | (m, 3H), 7.44-7.36 (m, 1H), 7.25 (d, J = 7.2 Hz, 1H), 7.10-7.05 (m, 1H), 6.95- |
| | | 6.88 (m, 1H), 6.85-6.83 (m, 1H), 6.67-6.61 (m, 2H), 6.21-6.16 (m, 1H), |
| | | 6.12 (br d, J = 3.6 Hz, 1H), 4.40-4.33 (m, 4H), 4.31-4.26 (m, 1H), 4.23 (s, |
| | | 3H), 3.89 (t, J = 6.8 Hz, 2H), 3.66-3.56 (m, 2H), 3.49-3.44 (m, 2H), 3.27- |
| | | 3.13 (m, 6H), 3.01-2.93 (m, 3H), 2.79-2.65 (m, 4H), 2.40-2.19 (m, 3H), 2.15- |
| | | 2.07 (m, 1H), 1.92-1.83 (m, 1H) |
| I-809 | 812.1 | 12.19-12.07 (m, 1H), 10.60-10.45 (m, 1H), 7.77-7.67 (m, 1H), 7.62-7.53 |
| | | (m, 2H), 7.45-7.30 (m, 2H), 7.19-7.12 (m, 3H), 7.10-7.03 (m, 1H), 7.02- |
| | | 6.94 (m, 1H), 6.90-6.84 (m, 1H), 6.23-6.09 (m, 2H), 4.42-4.31 (m, 4H), 4.30 |
| | | (s, 3H), 3.93-3.89 (m, 2H), 3.68-3.61 (m, 1H), 3.59 (br t, J = 5.6 Hz, 1H), |
| | | 3.25-3.15 (m, 4H), 3.14-3.01 (m, 6H), 3.00-2.96 (m, 2H), 2.80-2.73 (m, |
| | | 2H), 2.54 (br s, 4H), 2.35-2.26 (m, 2H) |
| I-812 | 755.3 | 1.40-1.65 (m, 1 H) 1.69-1.80 (m, 1 H) 1.81-1.98 (m, 8 H) 2.03 (br d, J = 7.6 |
| | | Hz, 3 H) 2.15 (br s, 4 H) 2.56 (br d, J = 2.4 Hz, 3 H) 2.80 (br t, J = 9.2 Hz, 4 H) |
| | | 2.97-3.19 (m, 8 H) 3.81 (s, 4 H) 4.23-4.50 (m, 2 H) 5.11 (br d, J = 6.4 Hz, 1 |
| | | H) 6.38 (br s, 1 H) 6.57 (br d, J = 14.0 Hz, 1 H) 6.67 (br d, J = 8.0 Hz, 1 H) 6.85- |
| | | 6.92 (m, 1 H) 7.18-7.25 (m, 2 H) 7.27 (s, 1 H) 10.76-10.93 (m, 1 H) 12.03 |
| | | (br d, J = 2.4 Hz, 1 H) |
| I-816 | 746.3 | 12.12 (s, 1H), 11.07 (d, J = 2.4 Hz, 1H), 7.24-7.19 (m, 2H), 7.17-7.13 (m, |
| | | 1H), 6.96 (t, J = 8.0 Hz, 1H), 6.88-6.79 (m, 1H), 6.74 (d, J = 8.0 Hz, 1H), 6.69 |
| | | (d, J = 8.4 Hz, 1H), 6.36 (s, 1H), 6.15 (d, J = 9.6 Hz, 1H), 5.32 (dd, J = 5.2, 12.8 |
| | | Hz, 1H), 4.36-4.31 (m, 2H), 3.97-3.93 (m, 2H), 3.64-3.60 (m, 4H), 3.59 (s, |
| | | 3H), 3.16-3.13 (m, 1H), 3.09-3.02 (m, 3H), 2.97 (s, 3H), 2.93-2.81 (m, 2H), |
| | | 2.72-2.61 (m, 3H), 2.37 (s, 1H), 2.26 (s, 1H), 2.14 (d, J = 6.0 Hz, 3H), 2.07 (d, |
| | | J = 6.0 Hz, 3H), 2.02-1.96 (m, 1H) |
| I-817 | 757.4 | 11.98 (br s, 1H), 10.98-10.37 (m, 1H), 7.33-7.23 (m, 1H), 7.07 (s, 1H), 7.01- |
| | | 6.85 (m, 3H), 6.76 (dd, J = 8.4, 14.4 Hz, 1H), 6.48 (d, J = 3.2 Hz, 1H), 6.21- |
| | | 6.07 (m, 1H), 5.50 (br d, J = 8.0 Hz, 1H), 4.42-4.29 (m, 3H), 3.75 (s, 3H), 3.67- |
| | | 3.54 (m, 2H), 3.30-3.26 (m, 2H), 3.24-2.95 (m, 6H), 2.79-2.65 (m, 4H), |
| | | 2.61-2.53 (m, 1H), 2.37 (br d, J = 3.2 Hz, 1H), 2.26 (br s, 1H), 2.10-1.99 (m, |
| | | 5H), 1.96-1.87 (m, 4H) |
| I-818 | 907.2 | 12.20-12.08 (s, 1H), 10.95-10.72 (s, 1H), 7.46-7.41 (m, 1H), 7.09-7.04 (d, |
| | | J = 8.4 Hz, 1H), 6.96-6.87 (m, 2H), 6.63-6.59 (d, J = 8.0 Hz, 1H), 6.57-6.52 |
| | | (m, 2H), 6.15-6.09 (m, 1H), 5.12-5.06 (d, J = 6.8 Hz, 1H), 4.35-4.30 (m, |
| | | 2H), 4.29-4.22 (m, 1H), 3.81-3.77 (s, 3H), 3.64-3.58 (m, 2H), 3.28-3.24 (s, |
| | | 6H), 3.22-3.20 (m, 2H), 3.16-3.12 (m, 2H), 3.08-3.04 (m, 2H), 2.81-2.76 |
| | | (m, 1H), 2.70-2.67 (s, 3H), 2.67-2.65 (m, 2H), 2.64-2.61 (s, 1H), 2.39- |
| | | 2.34 (s, 3H), 2.29-2.25 (s, 1H), 2.15-2.11 (m, 1H), 2.09-2.05 (m, 3H), 1.93- |
| | | 1.85 (m, 3H), 1.65-1.57 (m, 2H) |
| I-823 | 804.7 | 12.42-11.82 (m, 1H), 11.34-10.69 (m, 1H), 7.44-7.34 (m, 2H), 7.31-7.24 |
| | | (m, 1H), 6.98-6.82 (m, 4H), 6.62 (s, 1H), 6.13 (s, 1H), 5.78-5.65 (m, 1H), |
| | | 4.45-4.30 (m, 2H), 3.86 (d, J = 12.4 Hz, 2H), 3.67 (d, J = 6.4 Hz, 2H), 3.11- |
| | | 2.98 (m, 6H), 2.97-2.81 (m, 3H), 2.81-2.69 (m, 4H), 2.64 (s, 3H), 2.40-2.36 |
| | | (m, 2H), 2.28-2.19 (m, 2H), 1.91 (d, J = 12.0 Hz, 2H), 1.62 (d, J = 5.6 Hz, 3H), |
| | | 1.41-1.29 (m, 2H), 1.09-0.97 (m, 6H) |
| I-824 | 854.0 | 12.18-12.07 (m, 1H), 10.92-10.81 (m, 1H), 7.74-7.68 (m, 1H), 7.45-7.31 |
| | | (m, 2H), 7.17-7.04 (m, 2H), 6.96-6.85 (m, 1H), 6.71-6.56 (m, 2H), 6.51- |
| | | 6.43 (m, 1H), 6.23-6.10 (m, 2H), 5.21-5.09 (m, 1H), 4.43-4.35 (m, 3H), 4.33- |
| | | 4.24 (m, 2H), 3.82 (s, 3H), 3.67-3.56 (m, 2H), 3.43-3.37 (m, 4H), 3.21-3.12 |
| | | (m, 3H), 3.07 (br s, 6H), 3.01-2.94 (m, 3H), 2.85-2.77 (m, 1H), 2.59-2.55 |
| | | (m, 1H), 2.33 (br s, 1H), 2.30-2.22 (m, 1H), 2.19-2.10 (m, 1H), 2.00-1.89 |
| | | (m, 1H) |
| I-827 | 776.6 | 12.03 (br s, 1H), 11.11 (br s, 1H), 7.36-7.27 (m, 1H), 7.09 (s, 1H), 7.04-6.88 |
| | | (m, 5H), 6.50 (br s, 1H), 6.19-6.09 (m, 1H), 5.38 (br dd, J = 5.2, 12.4 Hz, 1H), |
| | | 4.39-4.29 (m, 2H), 3.80-3.69 (m, 6H), 3.62 (td, J = 5.6, 11.6 Hz, 2H), 3.27 (br |
| | | d, J = 11.6 Hz, 3H), 3.22-2.98 (m, 6H), 2.95-2.86 (m, 3H), 2.80-2.68 (m, |
| | | 2H), 2.66-2.60 (m, 1H), 2.38 (br d, J = 1.6 Hz, 1H), 2.27 (br d, J = 2.4 Hz, 1H), |
| | | 2.09 (d, J = 6.8 Hz, 3H), 1.99 (br s, 4H) |
| I-839 | 776.6 | 12.03 (br s, 1H), 11.11 (br s, 1H), 7.36-7.27 (m, 1H), 7.09 (s, 1H), 7.04-6.88 |
| | | (m, 5H), 6.50 (br s, 1H), 6.19-6.09 (m, 1H), 5.38 (br dd, J = 5.2, 12.4 Hz, 1H), |
| | | 4.39-4.29 (m, 2H), 3.80-3.69 (m, 6H), 3.62 (td, J = 5.6, 11.6 Hz, 2H), 3.27 (br |
| | | d, J = 11.6 Hz, 3H), 3.22-2.98 (m, 6H), 2.95-2.86 (m, 3H), 2.80-2.68 (m, |
| | | 2H), 2.66-2.60 (m, 1H), 2.38 (br d, J = 1.6 Hz, 1H), 2.27 (br d, J = 2.4 Hz, 1H), |
| | | 2.09 (d, J = 6.8 Hz, 3H), 1.99 (br s, 4H) |
| I-831 | 786.1 | 12.12 (d, J = 8.0 Hz, 1H), 10.76 (s, 1H), 7.75-7.67 (m, 1H), 7.55 (d, J = 8.0 |
| | | Hz, 2H), 7.46-7.36 (m, 1H), 7.10 (d, J = 8.8 Hz, 2H), 7.01-6.93 (m, 1H), 6.92- |
| | | 6.85 (m, 2H), 6.59-6.46 (m, 2H), 6.21-6.09 (m, 2H), 5.51 (d, J = 7.2 Hz, |
| | | 1H), 4.40-4.35 (m, 3H), 4.33-4.20 (m, 2H), 3.67-3.56 (m, 2H), 3.25-3.04 |
| | | (m, 6H), 3.02-2.94 (m, 3H), 2.91 (s, 4H), 2.83-2.65 (m, 2H), 2.57 (dd, J = 4.0, |
| | | 17.6 Hz, 2H), 2.40-2.25 (m, 3H), 2.22 (s, 3H), 2.14-2.06 (m, 1H), 1.92-1.80 |
| | | (m, 1H) |
| I-833 | 792.2 | 1.0-1.0 (m, 3 H), 1.0-1.1 (m, 3 H), 1.9-2.0 (m, 6 H), 2.2-2.3 (m, 1 H), 2.4- |
| | | 2.4 (m, 1 H), 2.7 (br s, 1 H), 2.8-2.8 (m, 1 H), 2.9-2.9 (m, 2 H), 2.9-3.0 (m, |
| | | 2 H), 3.0-3.2 (m, 6 H), 3.2 (br s, 2 H), 3.6-3.7 (m, 2 H), 3.7 (s, 3 H), 4.4-4.4 |
| | | (m, 2 H), 5.3-5.4 (m, 1 H), 6.1-6.2 (m, 1 H), 6.6-6.6 (m, 1 H), 6.9-6.9 (m, |
| | | 1 H), 7.0-7.0 (m, 3 H), 7.3-7.3 (m, 1 H), 7.3-7.4 (m, 1 H), 7.5-7.6 (m, 1 H), |
| | | 8.4-8.5 (m, 1 H), 11.0-11.2 (m, 1 H), 12.1-12.3 (m, 1 H) |
| I-836 | 837.2 | 10.85 (br s, 1H), 7.76-7.67 (m, 2H), 7.51 (d, J = 8.8 Hz, 2H), 7.45-7.30 (m, |
| | | 2H), 7.14 (br d, J = 8.8 Hz, 2H), 6.69 (d, J = 8.0 Hz, 1H), 6.60 (d, J = 14.4 Hz, |
| | | 1H), 6.27 (br s, 1H), 6.20 (td, J = 2.0, 13.6 Hz, 1H), 5.14 (d, J = 6.4 Hz, 1H), |
| | | 4.41-4.35 (m, 3H), 4.34 (br s, 1H), 4.32-4.25 (m, 1H), 3.83 (s, 3H), 3.69- |
| | | 3.56 (m, 2H), 3.38 (br d, J = 5.2 Hz, 4H), 3.23-3.05 (m, 9H), 3.03-2.91 (m, |
| | | 3H), 2.82 (ddd, J = 5.2 13.2, 17.6 Hz, 1H), 2.57 (br d, J = 3.2 Hz, 1H), 2.39- |
| | | 2.26 (m, 2H), 2.20-2.11 (m, 1H), 1.94 (dq, J = 4.8, 12.8 Hz, 1H) |
| I-837 | 772.1 | 12.20-12.04 (m, 1H), 11.16-10.89 (m, 1H), 7.32 (s, 1H), 7.27-7.25 (m, 2H), |
| | | 7.21-7.15 (m, 1H), 6.92-6.81 (m, 1H), 6.69 (d, J = 7.6 Hz, 1H), 6.41 (d, J = |
| | | 2.8 Hz, 1H), 6.15 (s, 1H), 5.70 (dd, J = 5.2, 11.6 Hz, 1H), 4.44-4.32 (m, 2H), |
| | | 3.67 (d, J = 5.6 Hz, 2H), 3.55-3.42 (m, 2H), 3.22-3.00 (m, 6H), 2.99-2.94 |
| | | (m, 1H), 2.92-2.81 (m, 3H), 2.78-2.72 (m, 2H), 2.68 (s, 4H), 2.42-2.32 (m, |
| | | 2H), 2.28-2.16 (m, 5H), 2.07-1.95 (m, 4H), 1.09-0.96 (m, 6H) |
| I-838 | 764.3 | 12.29-12.09 (m, 1H), 10.97-10.70 (m, 1H), 7.75-7.68 (m, 1H), 7.58 (br d, J = |
| | | 8.0 Hz, 2H), 7.44-7.35 (m, 2H), 7.34 (s, 1H), 7.06-6.95 (m, 1H), 6.84 (br s, |
| | | 1H), 6.72 (br d, J = 7.2 Hz, 1H), 6.50 (d, J = 12.0 Hz, 1H), 6.22-6.12 (m, 2H), |
| | | 5.27 (br d, J = 6.8 Hz, 1H), 4.40-4.34 (m, 3H), 4.34 (br s, 2H), 3.74 (s, 3H), |
| | | 3.65 (br t, J = 5.6 Hz, 1H), 3.58 (br t, J = 5.6 Hz, 1H), 3.22-3.03 (m, 6H), 3.00- |
| | | 2.95 (m, 2H), 2.91-2.86 (m, 2H), 2.85-2.77 (m, 3H), 2.56 (br d, J = 2.4 Hz, |
| | | 1H), 2.35-2.32 (m, 1H), 2.27 (br s, 1H), 2.18-2.10 (m, 1H), 1.93 (dq, J = 4.0, |
| | | 12.8 Hz, 1H) |
| I-839 | 819.2 | 12.19 (s, 1H), 10.85 (s, 1H), 7.76-7.68 (m, 1H), 7.62 (d, J = 7.6 Hz, 2H), 7.46- |
| | | 7.34 (m, 3H), 7.09-6.98 (m, 1H), 6.87 (s, 1H), 6.26 (d, J = 11.2 Hz, 1H), 6.22- |
| | | 6.13 (m, 2H), 6.08 (d, J = 11.2 Hz, 1H), 5.55 (d, J = 7.6 Hz, 1H), 4.41-4.32 |
| | | (m, 4H), 4.30 (s, 1H), 3.76 (s, 3H), 3.66-3.55 (m, 4H), 3.26-3.01 (m, 6H), |
| | | 2.98 (d, J = 8.0 Hz, 2H), 2.87-2.77 (m, 1H), 2.75-2.65 (m, 3H), 2.57 (s, 1H), |
| | | 2.36-2.25 (m, 2H), 2.15-2.08 (m, 1H), 2.03-1.84 (m, 5H) |
| I-840 | 817.3 | 12.16-12.06 (m, 1H), 10.58-10.45 (m, 1H), 7.63-7.51 (m, 2H), 7.50-7.46 |
| | | (m, 1H), 7.32 (d, J = 7.6 Hz, 1H), 7.13-7.01 (m, 3H), 6.56-6.51 (m, 1H), 4.51- |
| | | 4.42 (m, 1H), 4.31 (s, 3H), 3.92-3.81 (m, 3H), 3.39 (br d, J = 11.6 Hz, 2H), |
| | | 3.25-3.21 (m, 2H), 3.18-3.04 (m, 6H), 2.94-2.86 (m, 3H), 2.77 (t, J = 6.4 Hz, |
| | | 2H), 2.74-2.65 (m, 1H), 2.03 (br d, J = 9.2 Hz, 6H), 1.97-1.73 (m, 4H), 1.59- |
| | | 1.44 (m, 1H) |
| I-841 | 814.1 | 12.22 (dd, J = 1.6, 4.0 Hz, 1H), 11.05 (d, J = 2.4 Hz, 1H), 7.64-7.59 (m, 1H), |
| | | 7.53 (d, J = 8.0 Hz, 1H), 7.47 (s, 1H), 7.31-7.24 (m, 1H), 7.23-7.17 (m, 1H), |
| | | 7.08-6.95 (m, 1H), 6.69 (d, J = 7.2 Hz, 1H), 6.61-6.52 (m, 1H), 6.21-6.12 |
| | | (m, 1H), 5.70 (dd, J = 5.2, 11.6 Hz, 1H), 4.38-4.30 (m, 2H), 3.67-3.59 (m, |
| | | 2H), 3.51-3.45 (m, 2H), 3.23-3.00 (m, 6H), 2.99-2.77 (m, 5H), 2.75-2.71 |
| | | (m, 1H), 2.71-2.65 (m, 4H), 2.39 (d, J = 3.2 Hz, 1H), 2.31-2.16 (m, 2H), 2.08 |
| | | (d, J = 8.0 Hz, 4H), 2.03 (d, J = 16.4 Hz, 2H) |
| I-842 | 811.5 | 12.34-12.08 (m, 1H), 10.68-10.40 (m, 1H), 8.60-8.32 (m, 1H), 7.76-7.68 |
| | | (m, 1H), 7.67-7.61 (m, 2H), 7.51-7.45 (m, 2H), 7.43-7.35 (m, 1H), 7.35- |
| | | 7.29 (m, 1H), 7.12-7.01 (m, 3H), 6.91-6.85 (m, 1H), 6.22-6.13 (m, 2H), 4.37 |
| | | (br d, J = 7.6 Hz, 4H), 4.31 (s, 3H), 3.92-3.87 (m, 2H), 3.68-3.62 (m, 1H), |
| | | 3.60-3.57 (m, 1H), 3.22-3.07 (m, 6H), 3.01-2.97 (m, 2H), 2.92-2.89 (m, |
| | | 1H), 2.76 (br t, J = 6.4 Hz, 2H), 2.68-2.63 (m, 1H), 2.45-2.43 (m, 2H), 2.33 |
| | | (br s, 2H), 2.30-2.24 (m, 1H), 2.05-1.98 (m, 4H) |
| I-843 | 726.1 | 12.01-11.73 (m, 1H), 11.01-10.60 (m, 1H), 7.27 (d, J = 7.6 Hz, 1H), 7.10- |
| | | 7.03 (m, 3H), 7.01-6.92 (m, 3H), 6.50-6.45 (m, 1H), 4.54-4.44 (m, 1H), 3.89- |
| | | 3.80 (m, 2H), 3.77-3.73 (m, 3H), 3.53-3.47 (m, 2H), 3.22-3.05 (m, 6H), |
| | | 3.04-2.94 (m, 2H), 2.85-2.78 (m, 2H), 2.78-2.65 (m, 3H), 2.39-2.30 (m, |
| | | 1H), 2.26-2.17 (m, 1H), 2.06-2.02 (m, 3H), 1.99-1.88 (m, 6H), 1.86 (s, 2H), |
| | | 1.62-1.42 (m, 1H) |
| I-844 | 732.1 | 12.19 (s, 1H), 11.07 (s, 1H), 7.63-7.58 (m, 2H), 7.38 (d, J = 7.6 Hz, 2H), 7.08- |
| | | 6.99 (m, 1H), 6.96 (t, J = 8.0 Hz, 1H), 6.86 (s, 1H), 6.71 (dd, J = 8.0, 19.6 Hz, |
| | | 2H), 6.17 (s, 1H), 5.32 (dd, J = 5.2, 12.4 Hz, 1H), 4.35 (d, J = 7.2 Hz, 2H), 3.95 |
| | | (t, J = 6.0 Hz, 2H), 3.65-3.59 (m, 4H), 3.58 (s, 3H), 3.23-3.13 (m, 3H), 3.06 |
| | | (s, 3H), 3.01 (s, 1H), 2.96-2.84 (m, 2H), 2.70-2.59 (m, 2H), 2.38 (s, 2H), 2.27 |
| | | (s, 1H), 2.08 (d, J = 7.2 Hz, 3H), 2.02-1.95 (m, 1H) |
| I-847 | 888.3 | 11.98-11.88 (m, 1H), 10.81 (s, 1H), 7.73-7.69 (m, 1H), 7.44-7.36 (m, 1H), |
| | | 7.17 (d, J = 8.4 Hz, 1H), 7.04-6.98 (m, 2H), 6.97-6.93 (m, 1H), 6.88-6.82 |
| | | (m, 1H), 6.66 (s, 1H), 6.60-6.47 (m, 2H), 6.21-6.16 (m, 1H), 6.12-6.07 (m, |
| | | 1H), 4.36 (q, J = 7.2 Hz, 4H), 4.27 (s, 1H), 3.80 (dd, J = 5.2, 11.6 Hz, 1H), 3.72- |
| | | 3.70 (m, 3H), 3.64 (t, J = 5.6 Hz, 1H), 3.42 (d, J = 11.2 Hz, 4H), 3.24 (s, 3H), |
| | | 3.18-3.02 (m, 6H), 2.96-2.93 (m, 1H), 2.73-2.64 (m, 8H), 2.35-2.30 (m, |
| | | 2H), 2.27-2.15 (m, 2H), 2.00 (td, J = 4.4, 8.8 Hz, 1H), 1.92 (d, J = 10.8 Hz, |
| | | 2H), 1.67-1.58 (m, 2H) |
| I-848 | 824.6 | 12.33-11.98 (m, 1H), 11.18-10.63 (m, 1H), 7.79-7.68 (m, 1H), 7.56 (br d, J = |
| | | 8.4 Hz, 2H), 7.47-7.32 (m, 1H), 7.10 (dd, J = 8.8, 13.2 Hz, 3H), 7.03-6.93 |
| | | (m, 1H), 6.91-6.77 (m, 2H), 6.28-6.10 (m, 2H), 5.45 (br d, J = 7.2 Hz, 1H), |
| | | 4.47-4.27 (m, 5H), 3.71-3.55 (m, 2H), 3.39-3.36 (m, 2H), 3.28-3.14 (m, |
| | | 4H), 3.12-2.95 (m, 9H), 2.82-2.82 (m, 1H), 2.87-2.74 (m, 1H), 2.60-2.52 |
| | | (m, 2H), 2.37-2.25 (m, 2H), 2.15-2.02 (m, 2H) |
| I-850 | 804.2 | 12.13 (br s, 1H), 10.89 (s, 1H), 7.55 (d, J = 8.0 Hz, 1H), 7.42-7.35 (m, 1H), |
| | | 7.17 (d, J = 7.2 Hz, 1H), 7.03 (t, J = 7.2 Hz, 1H), 6.98-6.91 (m, 1H), 6.90 (s, |
| | | 1H), 6.87 (br d, J = 3.6 Hz, 1H), 6.62 (br d, J = 2.4 Hz, 1H), 6.13 (br s, 1H), |
| | | 4.42-4.33 (m, 3H), 4.22 (s, 3H), 3.86 (br d, J = 12.4 Hz, 2H), 3.67 (br d, J = |
| | | 5.6 Hz, 2H), 3.21-3.03 (m, 7H), 2.96 (dt, J = 6.4, 12.8 Hz, 2H), 2.78 (br t, J = |
| | | 11.2 Hz, 2H), 2.72-2.58 (m, 2H), 2.40-2.30 (m, 2H), 2.26 (br s, 1H), 2.22- |
| | | 2.13 (m, 1H), 1.91 (br d, J = 12.0 Hz, 2H), 1.70-1.58 (m, 3H), 1.41-1.29 (m, |
| | | 2H), 1.08-0.99 (m, 6H) |
| I-852 | 806.2 | 12.19 (s, 1H), 11.22-10.92 (m, 1H), 7.34 (t, J = 8.0 Hz, 1H), 7.16 (d, J = 8.0 |
| | | Hz, 1H), 7.05-6.97 (m, 2H), 6.96-6.80 (m, 2H), 6.44 (d, J = 1.2 Hz, 1H), 6.20- |
| | | 6.09 (m, 1H), 5.36 (dd, J = 5.6, 12.4 Hz, 1H), 4.51-4.31 (m, 2H), 3.70 (s, 3H), |
| | | 3.69-3.61 (m, 2H), 3.29-3.24 (m, 4H), 3.10-3.01 (m, 4H), 2.97-2.86 (m, |
| | | 5H), 2.74-2.64 (m, 4H), 2.40-2.32 (m, 2H), 2.29-2.22 (m, 1H), 2.08 (s, 3H), |
| | | 2.04-1.92 (m, 4H) |
| I-853 | 802.5 | 12.24-12.03 (m, 1H), 11.09 (br d, J = 0.8 Hz, 1H), 7.27 (s, 1H), 7.09 (s, 1H), |
| | | 7.05-6.97 (m, 2H), 6.90 (br d, J = 6.8 Hz, 1H), 6.87-6.78 (m, 1H), 6.38 (br s, |
| | | 1H), 6.14 (br s, 1H), 5.37 (br dd, J = 4.8, 12.4 Hz, 1H), 4.42-4.33 (m, 2H), 3.72 |
| | | (s, 3H), 3.66 (br d, J = 5.2 Hz, 2H), 3.26 (br d, J = 10.4 Hz, 2H), 3.15-3.03 (m, |
| | | 4H), 3.00-2.84 (m, 6H), 2.78-2.55 (m, 3H), 2.38-2.33 (m, 4H), 2.30-2.21 |
| | | (m, 1H), 2.13 (br s, 3H), 2.05-1.92 (m, 3H), 1.91-1.84 (m, 2H), 1.06-0.98 |
| | | (m, 6H) |
| I-854 | 785.4 | 11.60 (s, 1H), 11.10 (s, 1H), 7.75-7.67 (m, 1H), 7.45-7.34 (m, 2H), 7.28 (d, J = |
| | | 7.6 Hz, 1H), 7.07 (s, 1H), 7.04-7.01 (m, 1H), 7.00-6.96 (m, 2H), 6.91-6.83 |
| | | (m, 2H), 6.22-6.15 (m, 2H), 6.12-6.05 (m, 1H), 5.37 (dd, J = 5.6, 12.4 Hz, |
| | | 1H), 4.39-4.28 (m, 4H), 3.74 (d, J = 3.6 Hz, 3H), 3.70 (s, 3H), 3.64 (t, J = 5.6 |
| | | Hz, 1H), 3.57 (t, J = 5.6 Hz, 1H), 3.36 (d, J = 1.6 Hz, 1H), 3.26 (d, J = 11.2 Hz, |
| | | 2H), 2.97 (q, J = 7.2 Hz, 2H), 2.90-2.85 (m, 2H), 2.82-2.57 (m, 4H), 2.34- |
| | | 2.24 (m, 2H), 1.98 (s, 4H) |
| I-856 | 749.2 | 12.07-11.94 (m, 1H), 10.91-10.76 (m, 1H), 7.75-7.68 (m, 1H), 7.44-7.31 |
| | | (m, 2H), 7.25 (d, J = 7.6 Hz, 1H), 7.07 (d, J = 11.2 Hz, 1H), 7.02 (d, J = 7.6 Hz, |
| | | 1H), 6.97-6.92 (m, 2H), 6.91-6.85 (m, 1H), 6.45 (s, 1H), 6.22-6.16 (m, 1H), |
| | | 6.13-6.08 (m, 1H), 4.38-4.27 (m, 4H), 3.88 (dd, J = 4.8, 11.6 Hz, 1H), 3.68 |
| | | (d, J = 2.4 Hz, 3H), 3.64 (t, J = 5.6 Hz, 1H), 3.57 (t, J = 5.6 Hz, 1H), 3.20-3.02 |
| | | (m, 6H), 2.99-2.91 (m, 7H), 2.67-2.62 (m, 1H), 2.35-2.24 (m, 3H), 2.02 (td, |
| | | J = 4.4, 8.8 Hz, 1H) |
| I-857 | 820.2 | 12.22-12.14 (m, 1H), 10.88-10.82 (m, 1H), 8.16-8.07 (m, 1H), 7.70 (s, 1H), |
| | | 7.62 (br d, J = 7.2 Hz, 2H), 7.43 (br d, J = 7.6 Hz, 2H), 7.09-6.99 (m, 1H), 6.89- |
| | | 6.85 (m, 1H), 6.67 (br d, J = 7.6 Hz, 1H), 6.60-6.54 (m, 1H), 6.20-6.14 (m, |
| | | 1H), 5.11 (br d, J = 6.8 Hz, 1H), 4.66-4.58 (m, 2H), 4.41-4.32 (m, 2H), 4.30- |
| | | 4.23 (m, 1H), 3.81 (s, 3H), 3.69-3.58 (m, 2H), 3.16-3.05 (m, 6H), 2.85-2.77 |
| | | (m, 3H), 2.74-2.61 (m, 2H), 2.60-2.55 (m, 2H), 2.38-2.32 (m, 2H), 2.28 (br |
| | | dd, J = 1.6, 3.6 Hz, 1H), 2.17-2.10 (m, 1H), 1.98-1.80 (m, 6H) |
| I- | 791.3 | 12.20-12.11 (m, 1H), 10.81 (s, 1H), 8.15-8.07 (m, 1H), 7.72-7.64 (m, 1H), |
| 860 | | 7.61-7.56 (m, 2H), 7.12 (d, J = 8.8 Hz, 2H), 7.04 (br dd, J = 2.0, 11.6 Hz, 2H), |
| | | 7.01-6.95 (m, 2H), 6.85 (br s, 1H), 6.18-6.11 (m, 1H), 4.65-4.60 (m, 3H), |
| | | 4.40-4.32 (m, 2H), 3.81 (dd, J = 4.8, 11.6 Hz, 1H), 3.66 (br t, J = 5.6 Hz, 1H), |
| | | 3.60 (br t, J = 5.6 Hz, 1H), 3.29-3.13 (m, 6H), 3.12-3.07 (m, 4H), 3.00-2.95 |
| | | (m, 2H), 2.69-2.61 (m, 1H), 2.48-2.46 (m, 1H), 2.35 (br d, J = 2.0 Hz, 1H), |
| | | 2.30-2.24 (m, 1H), 2.20 (br dd, J = 3.6, 12.0 Hz, 1H), 2.16-2.10 (m, 2H), 2.02 |
| | | (dt, J = 4.8, 8.8 Hz, 1H), 1.87-1.79 (m, 2H) |
| I-861 | 820.5 | 12.21-11.99 (m, 1H), 10.78-10.59 (m, 1H), 7.79-7.66 (m, 1H), 7.62-7.53 |
| | | (m, 2H), 7.48-7.35 (m, 1H), 7.17-7.10 (m, 2H), 7.03-6.93 (m, 1H), 6.89- |
| | | 6.84 (m, 1H), 6.54-6.47 (m, 1H), 6.41 (dd, J = 2.4, 14.8 Hz, 1H), 6.23-6.12 |
| | | (m, 2H), 4.55-4.45 (m, 1H), 4.41-4.36 (m, 3H), 4.34-4.26 (m, 1H), 4.16- |
| | | 4.09 (m, 1H), 3.86-3.80 (m, 3H), 3.67-3.57 (m, 2H), 3.38-3.33 (m, 4H), 3.28- |
| | | 3.12 (m, 9H), 3.00-2.95 (m, 2H), 2.67-2.55 (m, 2H), 2.40-2.22 (m, 3H), |
| | | 2.13-2.03 (m, 1H), 1.97-1.81 (m, 1H) |
| I-863 | 730.3 | 12.20 (br s, 1H), 11.05 (br s, 1H), 7.68-7.62 (m, 2H), 7.48 (br d, J = 8.0 Hz, |
| | | 2H), 7.30-7.17 (m, 2H), 7.11-7.01 (m, 1H), 6.90 (d, J = 2.4 Hz, 1H), 6.69 (d, |
| | | J = 7.2 Hz, 1H), 6.18 (br s, 1H), 5.70 (dd, J = 5.2, 11.6 Hz, 1H), 4.36 (br d, J = |
| | | 8.0 Hz, 2H), 3.63 (td, J = 5.6, 11.6 Hz, 2H), 3.48 (br d, J = 9.6 Hz, 2H), 3.25- |
| | | 3.03 (m, 6H), 2.92-2.80 (m, 4H), 2.73 (br s, 1H), 2.71-2.65 (m, 4H), 2.39 (br |
| | | d, J = 3.2 Hz, 1H), 2.31-2.19 (m, 2H), 2.09 (d, J = 6.4 Hz, 3H), 2.05-1.97 (m, |
| | | 4H) |
| | 810.2 | 12.27-12.06 (m, 1H), 11.17-10.93 (m, 1H), 7.39-7.34 (m, 1H), 7.32-7.24 |
| | | (m, 2H), 7.17 (d, J = 8.4 Hz, 1H), 7.00-6.87 (m, 2H), 6.50 (d, J = 3.2 Hz, 1H), |
| | | 6.20-6.11 (m, 1H), 5.72 (dd, J = 4.8, 11.6 Hz, 1H), 4.37-4.29 (m, 2H), 3.61 |
| | | (td, J = 5.6, 11.2 Hz, 2H), 3.48 (d, J = 11.2 Hz, 2H), 3.22-2.96 (m, 8H), 2.91- |
| | | 2.81 (m, 1H), 2.80-2.68 (m, 4H), 2.64 (s, 3H), 2.37 (d, J = 3.2 Hz, 1H), 2.30- |
| | | 2.16 (m, 2H), 2.07 (d, J = 6.4 Hz, 3H), 1.98-1.86 (m, 2H), 1.70-1.52 (m, 3H), |
| | | 1.49-1.36 (m, 2H) |
| I-864 | 921.0 | 10.99-10.70 (m, 1H), 7.75-7.67 (m, 1H), 7.49-7.43 (m, 1H), 7.39-7.29 (m, |
| | | 2H), 7.16 (d, J = 7.6 Hz, 1H), 7.05 (s, 1H), 6.71-6.57 (m, 2H), 6.31-6.13 (m, |
| | | 2H), 5.15 (d, J = 6.8 Hz, 1H), 4.43-4.23 (m, 5H), 3.83 (s, 3H), 3.69-3.55 (m, |
| | | 2H), 3.44 (s, 4H), 3.23-2.92 (m, 12H), 2.88-2.77 (m, 1H), 2.57 (d, J = 3.2 Hz, |
| | | 1H), 2.38-2.27 (m, 2H), 2.19-2.11 (m, 1H), 2.01-1.88 (m, 1H) |
| I- | 855.3 | 11.00-10.66 (m, 1H), 7.71 (dd, J = 2.0, 11.6 Hz, 1H), 7.47-7.31 (m, 4H), 7.01- |
| 865 | | 6.92 (m, 2H), 6.70-6.55 (m, 2H), 6.31-6.13 (m, 2H), 5.14 (d, J = 6.8 Hz, |
| | | 1H), 4.39-4.25 (m, 5H), 3.82 (s, 3H), 3.67-3.56 (m, 2H), 3.42 (s, 4H), 3.14 |
| | | (dd, J = 3.2, 6.0 Hz, 2H), 3.07 (s, 6H), 3.02-2.93 (m, 3H), 2.81 (ddd, J = 5.2, |
| | | 12.8, 18.0 Hz, 1H), 2.59-2.52 (m, 2H), 2.37-2.26 (m, 2H), 2.17-2.08 (m, |
| | | 1H), 1.93 (dq, J = 4.0, 12.8 Hz, 1H) |
| I-866 | 867.5 | 11.16-10.39 (m, 1H), 7.71 (dd, J = 2.0, 10.8 Hz, 1H), 7.46-7.14 (m, 4H), 6.78- |
| | | 6.53 (m, 4H), 6.28-6.12 (m, 2H), 5.14 (d, J = 6.8 Hz, 1H), 4.41-4.24 (m, |
| | | 5H), 3.82 (s, 3H), 3.72 (d, J = 3.2 Hz, 3H), 3.67-3.56 (m, 2H), 3.40 (br s, 4H), |
| | | 3.22-3.13 (m, 2H), 3.09 (br s, 6H), 3.01-2.93 (m, 3H), 2.81 (ddd, J = 5.2, 12.8, |
| | | 17.6 Hz, 1H), 2.59-2.51 (m, 2H), 2.37-2.25 (m, 2H), 2.18-2.10 (m, 1H), 1.94 |
| | | (dq, J = 4.0, 12.8 Hz, 1H) |
| I-867 | 851.4 | 10.85 (s, 1H), 7.70 (d, J = 10.4 Hz, 1H), 7.44-7.33 (m, 1H), 7.30-7.12 (m, |
| | | 3H), 6.99 (s, 1H), 6.92 (d, J = 8.8 Hz, 1H), 6.67 (d, J = 8.0 Hz, 1H), 6.59 (d, J = |
| | | 14.0 Hz, 1H), 6.25 (s, 1H), 6.18 (d, J = 12.8 Hz, 1H), 5.13 (d, J = 6.4 Hz, 1H), |
| | | 4.41-4.24 (m, 5H), 3.82 (s, 3H), 3.66-3.54 (m, 2H), 3.37-3.34 (m, 4H), 3.17- |
| | | 2.92 (m, 12H), 2.86-2.76 (m, 1H), 2.57 (s, 1H), 2.36-2.24 (m, 2H), 2.18- |
| | | 2.04 (m, 4H), 2.00-1.87 (m, 1H) |
| I-868 | 838.3 | 10.85 (br s, 1H), 8.10 (d, J = 12.0 Hz, 1H), 7.72-7.66 (m, 2H), 7.50 (d, J = 8.8 |
| | | Hz, 2H), 7.41-7.30 (m, 1H), 7.13 (br d, J = 8.8 Hz, 2H), 6.69-6.56 (m, 2H), |
| | | 6.27 (br s, 1H), 5.14 (d, J = 6.8 Hz, 1H), 4.67-4.57 (m, 2H), 4.37 (br d, J = 14.4 |
| | | Hz, 2H), 4.31-4.24 (m, 1H), 3.82 (s, 3H), 3.68-3.58 (m, 2H), 3.37 (br d, J = |
| | | 4.4 Hz, 4H), 3.18 (br d, J = 2.0 Hz, 1H), 3.15-3.03 (m, 10H), 2.81 (ddd, J = |
| | | 5.2, 12.8, 17.6 Hz, 1H), 2.56 (br d, J = 3.2 Hz, 1H), 2.37 (br d, J = 2.0 Hz, 1H), |
| | | 2.29 (br d, J = 1.2 Hz, 1H), 2.18-2.09 (m, 1H), 1.93 (dq, J = 4.0, 12.8 Hz, 1H), |
| | | 1.35-1.12 (m, 1H) |
| I-869 | 811.1 | 12.14 (td, J = 3.6, 7.2 Hz, 1H), 11.32-10.81 (m, 1H), 7.31-7.19 (m, 3H), 7.05- |
| | | 6.95 (m, 2H), 6.88 (dd, J = 6.4, 14.4 Hz, 2H), 6.40 (d, J = 3.2 Hz, 1H), 6.19 (s, |
| | | 1H), 5.36 (dd, J = 5.2, 12.4 Hz, 1H), 4.75-4.30 (m, 2H), 3.98-3.83 (m, 1H), |
| | | 3.70 (s, 3H), 3.25 (d, J = 11.2 Hz, 2H), 3.21-2.97 (m, 6H), 2.94-2.85 (m, 3H), |
| | | 2.76-2.60 (m, 3H), 2.54 (s, 1H), 2.45-2.35 (m, 1H), 2.18 (s, 3H), 2.14-1.84 |
| | | (m, 6H), 1.78-1.41 (m, 4H) |
| I-870 | 800.1 | 12.38-11.84 (m, 1H), 11.07 (d, J = 2.0 Hz, 1H), 7.33-7.17 (m, 3H), 7.05-6.93 |
| | | (m, 2H), 6.92-6.79 (m, 2H), 6.39 (d, J = 2.8 Hz, 1H), 6.12 (s, 1H), 5.37 (dd, J = |
| | | 5.6, 12.8 Hz, 1H), 4.40-4.17 (m, 2H), 3.70 (s, 3H), 3.63 (t, J = 5.6 Hz, 1H), |
| | | 3.50-3.40 (m, 2H), 3.24 (d, J = 11.2 Hz, 2H), 3.21-2.96 (m, 6H), 2.94-2.84 |
| | | (m, 3H), 2.77-2.60 (m, 3H), 2.30 (s, 1H), 2.24 (d, J = 7.2 Hz, 2H), 2.19-2.15 |
| | | (m, 4H), 2.14-2.11 (m, 1H), 2.10-1.99 (m, 2H), 1.98-1.92 (m, 4H), 1.92- |
| | | 1.81 (m, 1H), 1.81-1.70 (m, 1H) |
| I-871 | 786.7 | 0.70-0.78 (m, 4 H) 1.91-2.04 (m, 6 H) 2.15-2.37 (m, 4 H) 2.23-2.30 (m, 1 |
| | | H) 2.63-2.75 (m, 3 H) 2.83-2.92 (m, 3 H) 3.00-3.19 (m, 6 H) 3.25 (br d, J = |
| | | 12.0 Hz, 3 H) 3.70 (s, 3 H) 3.83-3.89 (m, 1 H) 4.38 (br s, 1 H) 4.58 (br d, J = |
| | | 1.2 Hz, 1 H) 5.37 (br dd, J = 12, 5.2 Hz, 1 H) 6.13-6.20 (m, 1 H) 6.39 (d, J = |
| | | 3.2 Hz, 1 H) 6.89 (br d, J = 7.6 Hz, 1 H) 6.82 (br d, J = 6.0 Hz, 1 H) 6.95-7.04 |
| | | (m, 2 H) 7.22-7.32 (m, 3 H) 10.96-11.20 (m, 1 H) 12.03-12.23 (m, 1 H) |
| I-872 | 793.5 | 12.20-12.09 (m, 1H), 11.13-11.06 (m, 1H), 7.48-7.40 (m, 1H), 6.95 (s, 6H), |
| | | 6.62 (d, J = 2.0 Hz, 1H), 6.16-6.10 (m, 1H), 5.41-5.33 (m, 1H), 4.42-4.32 |
| | | (m, 2H), 3.90-3.58 (m, 7H), 3.27-3.00 (m, 11H), 2.98 (s, 3H), 2.77-2.68 (m, |
| | | 1H), 2.64 (s, 1H), 2.39-2.23 (m, 2H), 2.07-1.98 (m, 1H), 1.06-0.99 (m, 6H) |
| I-873 | 789.1 | 12.23-11.96 (m, 1H), 11.28-10.89 (m, 1H), 7.23-7.13 (m, 1H), 7.06-6.98 |
| | | (m, 3H), 6.93 (dd, J = 3.2, 5.6 Hz, 2H), 6.85-6.77 (m, 1H), 6.44-6.36 (m, 1H), |
| | | 6.14 (s, 1H), 5.38 (dd, J = 5.6, 12.4 Hz, 1H), 4.42-4.32 (m, 2H), 3.94-3.70 (m, |
| | | 2H), 3.70-3.64 (m, 5H), 3.25-3.13 (m, 4H), 3.13-3.01 (m, 6H), 3.01-2.86 |
| | | (m, 4H), 2.79-2.69 (m, 1H), 2.68-2.61 (m, 1H), 2.39-2.35 (m, 1H), 2.32- |
| | | 2.19 (m, 1H), 2.18-2.11 (m, 3H), 2.07-1.98 (m, 1H), 1.07-0.99 (m, 6H) |
| I-874 | 888.2 | 11.95 (s, 1H), 11.09 (s, 1H), 7.18 (d, J = 8.0 Hz, 1H), 7.01-6.95 (m, 1H), 6.94- |
| | | 6.90 (m, 1H), 6.90-6.83 (m, 2H), 6.66 (s, 1H), 6.60 (d, J = 8.8 Hz, 1H), 6.53- |
| | | 6.46 (m, 1H), 6.10 (s, 1H), 5.35 (dd, J = 5.2, 12.4 Hz, 1H), 4.40-4.30 (m, 2H), |
| | | 3.72 (s, 3H), 3.65 (s, 5H), 3.25 (s, 6H), 3.18 (d, J = 10.4 Hz, 3H), 3.09-3.02 |
| | | (m, 2H), 3.00-2.84 (m, 3H), 2.72 (s, 6H), 2.68-2.58 (m, 2H), 2.44-2.32 (m, |
| | | 3H), 2.25 (d, J = 1.6 Hz, 1H), 1.96 (d, J = 10.8 Hz, 2H), 1.73-1.62 (m, 2H), |
| | | 1.06-1.00 (m, 6H) |
| I-877 | 852.0 | 12.25-12.05 (m, 1H), 10.93-10.75 (m, 1H), 7.74-7.68 (m, 1H), 7.45-7.34 |
| | | (m, 1H), 7.09-7.02 (m, 2H), 6.89-6.80 (m, 1H), 6.73-6.68 (d, J = 8.4 Hz, |
| | | 1H), 6.61-6.55 (d, J = 14.4 Hz, 1H), 6.44-6.40 (m, 1H), 6.21-6.12 (m, 2H), |
| | | 5.16-5.10 (d, J = 6.4 Hz, 1H), 4.39-4.36 (m, 2H), 4.35-4.33 (m, 1H), 4.31- |
| | | 4.24 (m, 2H), 3.84-3.80 (s, 3H), 3.67-3.61 (m, 1H), 3.58-3.55 (m, 1H), |
| | | 3.30-3.27 (m, 2H), 3.25-3.20 (m, 5H), 3.13-3.08 (s, 6H), 3.00-2.94 (m, |
| | | 3H), 2.84-2.76 (m, 1H), 2.59-2.53 (m, 2H), 2.36-2.29 (m, 2H), 2.28-2.23 |
| | | (m, 1H), 2.12-2.09 (s, 3H), 1.97-1.88 (m, 1H) |
| I-878 | 776.1 | 12.19 (s, 1H), 11.11 (s, 1H), 7.90 (d, J = 8.8 Hz, 1H), 7.10-6.90 (m, 5H), 6.87 |
| | | (s, 1H), 6.21-6.17 (m, 1H), 5.45-5.36 (dd, J = 12.4, 5.2 Hz), 4.56-4.38 (m, 4H), |
| | | 3.71 (s, 3H), 3.71-3.61 (m, 2H), 3.40-2.84 (m, 14H), 2.80-2.57 (m, 2H), 2.48- |
| | | 2.24 (m, 3H), 2.05-1.98 (m, 1H), 1.10-0.98 (m, 6H) |
| I-879 | 824.6 | 12.20-11.86 (m, 1H), 11.16-11.02 (m, 1H), 7.19-7.07 (m, 2H), 7.04-6.96 |
| | | (m, 3H), 6.94-6.88 (m, 1H), 6.51 (br s, 1H), 5.45-5.33 (m, 1H), 4.55-4.42 |
| | | (m, 1H), 4.08-3.93 (m, 1H), 3.74 (d, J = 16.4 Hz, 6H), 3.31-3.25 (m, 7H), |
| | | 3.25-2.98 (m, 10H), 2.97-2.83 (m, 5H), 2.82-2.54 (m, 4H), 2.21-1.71 (m, |
| | | 10H), 1.58-1.42 (m, 1H), 1.07 (br t, J = 6.4 Hz, 3H), 0.99 (br dd, J = 6.8, 14.4 |
| | | Hz, 3H) |
| I-880 | 849.3 | 12.11-11.82 (m, 1H), 11.00-10.59 (m, 1H), 7.76-7.63 (m, 1H), 7.45-7.34 |
| | | (m, 1H), 7.32-7.22 (m, 1H), 7.12-7.03 (m, 1H), 7.01-6.86 (m, 2H), 6.73- |
| | | 6.64 (m, 1H), 6.62-6.52 (m, 1H), 6.50-6.42 (m, 1H), 6.23-6.06 (m, 2H), 5.11 |
| | | (br d, J = 6.4 Hz, 1H), 4.39-4.33 (m, 3H), 4.31-4.20 (m, 2H), 3.81 (s, 3H), |
| | | 3.75 (d, J = 3.6 Hz, 3H), 3.67-3.61 (m, 1H), 3.60-3.55 (m, 1H), 3.22-3.04 |
| | | (m, 6H), 2.97 (q, J = 7.2 Hz, 3H), 2.68 (br d, J = 9.2 Hz, 5H), 2.56 (br d, J = 2.4 |
| | | Hz, 1H), 2.37-2.30 (m, 2H), 2.26 (br s, 1H), 2.18-2.10 (m, 1H), 1.95-1.90 |
| | | (m, 4H) |
| I-881 | 924.2 | 11.12-11.07 (m, 1H), 8.56-8.38 (m, 1H), 8.16-8.11 (m, 1H), 7.72-7.67 (m, |
| | | 1H), 7.53-7.45 (m, 1H), 7.43-7.39 (m, 1H), 7.38-7.33 (m, 1H), 7.05-6.96 |
| | | (m, 3H), 6.92-6.86 (m, 3H), 6.21-6.13 (m, 1H), 5.41-5.35 (m, 1H), 4.42 (br |
| | | s, 1H), 4.42-4.33 (m, 1H), 3.89-3.80 (m, 2H), 3.63-3.54 (m, 6H), 3.02-2.95 |
| | | (m, 8H), 2.91-2.87 (m, 3H), 2.84-2.71 (m, 4H), 2.70-2.64 (m, 2H), 2.41- |
| | | 2.33 (m, 2H), 2.04-1.99 (m, 1H), 1.78-1.74 (m, 2H), 1.46-1.37 (m, 2H), 1.28- |
| | | 1.22 (m, 1H). |
| I-882 | 811.2 | 12.11 (d, J = 6.0 Hz, 1H), 10.54 (s, 1H), 7.74-7.69 (m, 1H), 7.55 (d, J = 8.4 Hz, |
| | | 2H), 7.49 (d, J = 8.0 Hz, 1H), 7.45-7.36 (m, 1H), 7.31 (d, J = 7.2 Hz, 1H), 7.15- |
| | | 7.11 (m, 2H), 7.11-7.07 (m, 1H), 7.01-6.94 (m, 1H), 6.88 (s, 1H), 6.22-6.17 |
| | | (m, 1H), 6.14 (s, 1H), 4.39-4.36 (m, 3H), 4.27 (s, 3H), 3.97 (d, J = 11.6 Hz, |
| | | 2H), 3.89 (t, J = 6.8 Hz, 2H), 3.67-3.56 (m, 3H), 3.27-3.05 (m, 6H), 3.04- |
| | | 2.94 (m, 5H), 2.77 (t, J = 6.8 Hz, 2H), 2.35-2.26 (m, 2H), 2.08-2.02 (m, 2H), |
| | | 1.97-1.87 (m, 2H) |
| I-883 | 825.4 | 12.18 (br d, J = 7.6 Hz, 1H), 10.58-10.49 (m, 1H), 7.75-7.68 (m, 1H), 7.64- |
| | | 7.58 (m, 2H), 7.43-7.26 (m, 4H), 7.08-6.98 (m, 3H), 6.90-6.84 (m, 1H), 6.22- |
| | | 6.13 (m, 2H), 4.40-4.35 (m, 3H), 4.33-4.29 (m, 1H), 4.25 (s, 3H), 3.91-3.85 |
| | | (m, 2H), 3.67-3.60 (m, 1H), 3.60-3.57 (m, 1H), 3.25 (br d, J = 11.6 Hz, 4H), |
| | | 3.20-3.00 (m, 6H), 3.00-2.94 (m, 2H), 2.78-2.73 (m, 2H), 2.69-2.66 (m, |
| | | 2H), 2.38-2.27 (m, 2H), 1.85-1.71 (m, 3H), 1.59-1.48 (m, 2H) |
| I-884 | 708.4 | 12.14 (s, 1H), 10.82 (s, 1H), 7.29 (s, 1H), 7.26-7.19 (m, 2H), 7.09-7.05 (m, |
| | | 1H), 7.05-7.02 (m, 1H), 7.01-6.97 (m, 1H), 6.91-6.80 (m, 1H), 6.39 (br s, |
| | | 1H), 6.19-6.12 (m, 1H), 4.39-4.30 (m, 2H), 3.82 (dd, J = 4.8, 11.7 Hz, 1H), |
| | | 3.65-3.57 (m, 2H), 3.50 (br d, J = 11.2 Hz, 2H), 3.23-2.98 (m, 6H), 2.82 (br |
| | | t, J = 10.4 Hz, 2H), 2.73-2.62 (m, 2H), 2.37 (br d, J = 3.2 Hz, 1H), 2.29-2.19 |
| | | (m, 2H), 2.18-2.15 (m, 3H), 2.08 (d, J = 5.2 Hz, 3H), 2.03 (dt, J = 4.4, 9.0 Hz, |
| | | 1H), 1.99-1.82 (m, 5H) |
| I-885 | 745.1 | 12.18-12.10 (s, 1H), 10.59-10.49 (s, 1H), 7.34-7.30 (m, 2H), 7.27-7.24 |
| | | (s, 2H), 7.11-7.02 (m, 2H), 6.91-6.81 (m, 1H), 6.41-6.37 (d, J = 1.6 Hz, |
| | | 1H), 6.18-6.13 (m, 1H), 4.39-4.34 (m, 2H), 4.32-4.29 (s, 3H), 3.93-3.87 |
| | | (t, J = 6.4 Hz, 2H), 3.64-3.58 (m, 2H), 3.40-3.65 (d, J = 11.2 Hz, 2H), 3.18- |
| | | 3.00 (m, 6H), 2.93-2.86 (m, 2H), 2.79-2.73 (t, J = 6.8 Hz, 3H), 2.38-2.35 |
| | | (m, 1H), 2.29-2.23 (s, 1H), 2.20-2.16 (m, 3H), 2.09-2.06 (m, 3H), 2.04- |
| | | 2.01 (m, 2H), 2.01-1.97 (m, 2H) |
| I-887 | 903.1 | 12.30-11.88 (m, 1H), 10.84 (s, 1H), 7.77-7.67 (m, 1H), 7.53 (d, J = 8.8 Hz, |
| | | 2H), 7.45-7.34 (m, 1H), 7.06 (d, J = 8.8 Hz, 2H), 7.01-6.92 (m, 1H), 6.86 (s, |
| | | 1H), 6.27-6.22 (m, 1H), 6.22-6.17 (m, 1H), 6.14 (s, 1H), 6.07-6.01 (m, 1H), |
| | | 5.54 (d, J = 7.6 Hz, 1H), 4.41-4.27 (m, 5H), 3.72 (s, 3H), 3.66 (t, J = 5.6 Hz, |
| | | 1H), 3.61-3.52 (m, 3H), 3.23 (s, 6H), 3.14-3.03 (m, 3H), 3.01-2.95 (m, 2H), |
| | | 2.86-2.75 (m, 1H), 2.73-2.67 (m, 4H), 2.61-2.58 (m, 1H), 2.55-2.53 (m, |
| | | 2H), 2.41-2.35 (m, 2H), 2.27 (s, 1H), 2.14-2.07 (m, 1H), 2.02-1.91 (m, 3H), |
| | | 1.70-1.48 (m, 3H) |
| I-888 | 810.3 | 12.18 (s, 1H), 10.88 (s, 1H), 7.54 (d, J = 8.0 Hz, 1H), 7.29 (d, J = 13.2 Hz, 1H), |
| | | 7.17 (d, J = 7.2 Hz, 2H), 7.03 (t, J = 7.2 Hz, 1H), 7.00-6.88 (m, 1H), 6.50 (s, |
| | | 1H), 6.14 (s, 1H), 4.40-4.28 (m, 3H), 4.22 (s, 3H), 3.65-3.56 (m, 2H), 3.48 (d, |
| | | J = 11.2 Hz, 2H), 3.23-2.93 (m, 8H), 2.76 (t, J = 11.6 Hz, 2H), 2.69-2.60 (m, |
| | | 2H), 2.38-2.25 (m, 3H), 2.17 (dd, J = 5.6, 12.4 Hz, 1H), 2.07 (d, J = 5.6 Hz, |
| | | 3H), 1.93 (d, J = 11.6 Hz, 2H), 1.73-1.53 (m, 3H), 1.43 (q, J = 10.8 Hz, 2H) |
| I-889 | 800.3 | 11.39-11.31 (m, 1H), 11.16-11.03 (m, 1H), 8.14-8.06 (m, 1H), 7.73-7.64 |
| | | (m, 1H), 7.25 (d, J = 7.6 Hz, 1H), 7.07-6.95 (m, 4H), 6.90 (d, J = 7.6 Hz, 1H), |
| | | 6.86-6.76 (m, 1H), 6.12-6.03 (m, 1H), 5.91 (s, 1H), 5.37 (dd, J = 5.2, 12.4 Hz, |
| | | 1H), 4.67-4.56 (m, 2H), 4.38-4.27 (m, 2H), 3.76-3.68 (m, 6H), 3.64 (t, J = |
| | | 5.6 Hz, 1H), 3.59 (t, J = 5.6 Hz, 1H), 3.27 (s, 2H), 3.24 (d, J = 0.8 Hz, 1H), 3.12- |
| | | 3.06 (m, 2H), 2.88 (d, J = 11.6 Hz, 2H), 2.81-2.58 (m, 4H), 2.36 (s, 3H), 2.33 |
| | | (d, J = 1.6 Hz, 1H), 2.26 (d, J = 2.0 Hz, 1H), 1.98 (d, J = 5.6 Hz, 4H) |
| I-890 | 787.2 | 13.71-13.50 (s, 1H), 11.19-11.02 (s, 1H), 8.13-8.07 (d, J = 11.2 Hz, 1H), |
| | | 7.90-7.78 (s, 1H), 7.73-7.64 (d, J = 11.6 Hz, 1H), 7.36-7.32 (d, J = 7.6 Hz, |
| | | 1H), 7.12-7.09 (s, 1H), 7.05-7.01 (m, 2H), 7.00-6.95 (m, 2H), 6.92-6.87 |
| | | (d, J = 7.2 Hz, 1H), 6.23-6.15 (m, 1H), 5.40-5.33 (m, 1H), 4.65-4.59 (m, |
| | | 2H), 4.40-4.31 (m, 2H), 3.79-3.76 (d, J = 3.6 Hz, 3H), 3.72-3.68 (s, 3H), |
| | | 3.68-3.64 (m 1H), 3.62-3.59 (m, 1H), 3.28-3.24 (m, 2H), 3.13-3.07 (m, |
| | | 2H), 2.93-2.84 (m, 3H), 2.80-2.69 (m, 2H), 2.65-3.60 (m, 1H), 2.37-2.26 |
| | | (m, 2H), 2.04-2.00(m, 2H), 1.99-1.94(m, 3H) |
| I-897 | 750.0 | 12.06-11.95 (m, 1H), 10.93-10.77 (m, 1H), 8.14-8.08 (m, 1H), 7.74-7.62 |
| | | (m, 1H), 7.37-7.21 (m, 2H), 7.11-6.99 (m, 2H), 6.97-6.86 (m, 3H), 6.45 (s, |
| | | 1H), 6.16-6.06 (m, 1H), 4.66-4.58 (m, 2H), 4.39-4.29 (m, 2H), 3.95-3.85 |
| | | (m, 1H), 3.73-3.62 (m, 4H), 3.59 (t, J = 5.2 Hz, 1H), 3.24-3.00 (m, 9H), 2.99- |
| | | 2.90 (m, 4H), 2.72-2.63 (m, 1H), 2.35-2.20 (m, 3H), 2.02 (d, J = 4.4, 8.8 Hz, |
| | | 1H). |
| I-898 | 774.6 | 12.21-12.02 (m, 1H), 11.09 (s, 1H), 7.32 (s, 1H), 7.26-7.17 (m, 2H), 7.04- |
| | | 6.94 (m, 2H), 6.92-6.78 (m, 2H), 6.34 (d, J = 2.4 Hz, 1H), 6.19-6.11 (m, 1H), |
| | | 5.40-5.31 (m, 1H), 4.34 (d, J = 6.4 Hz, 2H), 3.70 (s, 3H), 3.64-3.56 (m, 2H), |
| | | 3.32-3.30 (m, 6H), 3.25 (d, J = 11.2 Hz, 2H), 3.19-2.97 (m, 6H), 2.90-2.85 |
| | | (m, 2H), 2.77-2.67 (m, 2H), 2.33-2.23 (m, 1H), 2.07 (d, J = 5.2 Hz, 3H), 2.00- |
| | | 1.94 (m, 4H), 0.98 (q, J = 7.2 Hz, 3H) |
| I-899 | 802.2 | 12.28-11.98 (m, 1H), 11.26-10.92 (m, 1H), 7.32 (s, 1H), 7.21 (s, 2H), 7.04- |
| | | 6.96 (m, 2H), 6.91-6.79 (m, 2H), 6.38-6.30 (m, 1H), 6.19-6.10 (m, 1H), 5.41- |
| | | 5.29 (m, 1H), 4.41-4.33 (m, 2H), 3.70 (s, 3H), 3.68-3.62 (m, 2H), 3.25 (d, J = |
| | | 11.2 Hz, 4H), 3.16-3.03 (m, 5H), 2.99-2.82 (m, 5H), 2.78-2.68 (m, 2H), |
| | | 2.66-2.60 (m, 1H), 2.37-2.24 (m, 2H), 1.98 (d, J = 6.0 Hz, 5H), 1.04 (d, J = |
| | | 6.8 Hz, 4H), 1.01-0.95 (m, 5H) |
| I-900 | 804.3 | 12.29-12.05 (m, 1H), 11.08 (s, 1H), 7.40 (d, J = 7.6 Hz, 1H), 7.28-7.19 (m, |
| | | 2H), 7.13-7.04 (m, 1H), 7.03-6.97 (m, 2H), 6.95-6.87 (m, 2H), 6.17 (s, 1H), |
| | | 5.36 (dd, J = 5.6, 12.8 Hz, 1H), 4.46-4.33 (m, 2H), 3.91 (s, 3H), 3.76-3.59 (m, |
| | | 5H), 3.24 (d, J = 10.8 Hz, 4H), 3.16-3.03 (m, 4H), 2.99-2.85 (m, 4H), 2.76- |
| | | 2.55 (m, 3H), 2.42-2.22 (m, 2H), 2.05-1.86 (m, 5H), 1.08-0.98 (m, 6H) |
| I-901 | 802.5 | 12.13 (s, 1H), 11.10 (s, 1H), 7.26 (d, J = 8.0 Hz, 1H), 7.13 (d, J = 8.0 Hz, 1H), |
| | | 7.05-6.96 (m, 2H), 6.90 (d, J = 6.4 Hz, 1H), 6.86-6.76 (m, 1H), 6.37 (s, 1H), |
| | | 6.14 (s, 1H), 5.37 (dd, J = 5.2, 12.4 Hz, 1H), 4.38 (d, J = 13.2 Hz, 2H), 3.70 (s, |
| | | 3H), 3.68-3.62 (m, 2H), 3.30 (s, 3H), 3.25 (d, J = 9.2 Hz, 2H), 3.12 (d, J = 4.0 |
| | | Hz, 2H), 3.04 (d, J = 10.8 Hz, 2H), 3.00-2.92 (m, 4H), 2.91-2.84 (m, 1H), |
| | | 2.77-2.68 (m, 1H), 2.68-2.62 (m, 1H), 2.36 (s, 1H), 2.33 (s, 3H), 2.08 (s, 3H), |
| | | 2.04-1.94 (m, 2H), 1.89 (d, J = 3.2 Hz, 3H), 1.06-0.98 (m, 6H) |
| I-903 | 910.3 | 12.11 (br d, J = 8.8 Hz, 1H), 11.12-11.04 (m, 1H), 7.75-7.68 (m, 1H), 7.53 (br |
| | | d, J = 8.4 Hz, 2H), 7.44-7.36 (m, 1H), 7.06 (br d, J = 8.4 Hz, 2H), 7.00-6.96 |
| | | (m, 1H), 6.96-6.90 (m, 2H), 6.89-6.84 (m, 2H), 6.22-6.10 (m, 2H), 5.39- |
| | | 5.29 (m, 1H), 4.39-4.34 (m, 3H), 4.30 (br s, 1H), 3.66-3.56 (m, 5H), 3.25- |
| | | 3.15 (m, 9H), 3.10-3.03 (m, 2H), 3.01-2.92 (m, 3H), 2.91-2.83 (m, 1H), 2.75- |
| | | 2.68 (m, 6H), 2.67 (br s, 2H), 2.36-2.23 (m, 3H), 2.03-1.91 (m, 3H), 1.75- |
| | | 1.60 (m, 2H) |
| I-905 | 855.9 | 12.33-12.20 (m, 1H), 10.90-10.81 (m, 1H), 7.75-7.68 (m, 1H), 7.44-7.34 |
| | | (m, 3H), 7.08 (dd, J = 5.6, 20.0 Hz, 1H), 6.92 (d, J = 2.4 Hz, 1H), 6.69 (d, J = |
| | | 8.0 Hz, 1H), 6.58 (d, J = 14.0 Hz, 1H), 6.19 (d, J = 12.0 Hz, 2H), 5.13 (d, J = |
| | | 6.4 Hz, 1H), 4.40-4.35 (m, 3H), 4.34-4.25 (m, 2H), 3.82 (s, 3H), 3.67-3.56 |
| | | (m, 2H), 3.21 (s, 6H), 3.06 (s, 6H), 3.02-2.92 (m, 3H), 2.86-2.77 (m, 1H), |
| | | 2.56 (d, J = 2.0 Hz, 2H), 2.35-2.26 (m, 2H), 2.18-2.11 (m, 1H), 1.93 (q, J = |
| | | 4.4, 12.4 Hz, 1H). |
| I-907 | 822.1 | 12.16 (d, J = 6.4 Hz, 1H), 10.79 (s, 1H), 7.72 (d, J = 1.6 Hz, 1H), 7.47-7.31 (m, |
| | | 1H), 7.11-7.01 (m, 2H), 6.96-6.80 (m, 2H), 6.56 (d, J = 1.6 Hz, 1H), 6.49- |
| | | 6.41 (m, 2H), 6.23-6.10 (m, 2H), 5.86 (d, J = 7.6 Hz, 1H), 4.37 (q, J = 7.2 Hz, |
| | | 3H), 4.33-4.24 (m, 2H), 3.66-3.56 (m, 2H), 3.22 (s, 4H), 3.05 (s, 6H), 3.01- |
| | | 2.93 (m, 3H), 2.71 (s, 1H), 2.60 (d, J = 4.0 Hz, 1H), 2.58-2.53 (m, 1H), 2.49- |
| | | 2.37 (m, 2H), 2.35-2.25 (m, 2H), 2.12 (s, 3H), 2.09 (s, 1H), 1.93-1.77 (m, 1H) |
| I-906 | 888.7 | 11.70 (s, 1H), 11.16-11.05 (m, 1H), 8.16-8.10 (m, 1H), 7.75-7.64 (m, 1H), |
| | | 7.58 (d, J = 8.4 Hz, 2H), 7.39-7.32 (m, 1H), 7.19-7.14 (m, 1H), 7.08 (d, J = |
| | | 8.8 Hz, 3H), 7.05 (s, 2H), 6.87 (d, J = 2.4 Hz, 1H), 6.33 (s, 1H), 5.43-5.33 (m, |
| | | 1H), 4.68-4.60 (m, 2H), 4.45-4.36 (m, 2H), 3.77-3.67 (m, 2H), 3.64 (t, J = |
| | | 5.6 Hz, 1H), 3.61-3.57 (m, 4H), 3.22 (s, 6H), 3.17-3.13 (m, 2H), 3.11 (t, J = |
| | | 7.2 Hz, 2H), 2.95-2.85 (m, 3H), 2.75 (s, 4H), 2.69 (s, 1H), 2.68-2.66 (m, 1H), |
| | | 2.62 (d, J = 3.6 Hz, 1H), 2.36-2.31 (m, 2H), 2.31-2.22 (m, 1H), 2.04-1.93 |
| | | (m, 1H) |
| I-910 | 792.3 | 11.90 (br s, 1H), 11.25-10.95 (m, 1H), 7.13 (s, 1H), 7.06-6.99 (m, 3H), 6.97- |
| | | 6.89 (m, 2H), 6.47 (d, J = 2.8 Hz, 1H), 5.38 (br dd, J = 5.2, 12.4 Hz, 1H), 4.48 |
| | | (br t, J = 9.6 Hz, 1H), 3.87 (br d, J = 12.0 Hz, 1H), 3.74 (s, 6H), 3.28 (br d, J = |
| | | 10.4 Hz, 3H), 3.21-3.04 (m, 6H), 3.00-2.87 (m, 5H), 2.80-2.54 (m, 4H), 2.34 |
| | | (s, 3H), 2.08-1.98 (m, 6H), 1.95-1.77 (m, 5H) |
| I-911 | 835.4 | 12.02 (br d, J = 8.4 Hz, 1H), 10.81 (s, 1H), 8.15-8.04 (m, 1H), 7.73-7.63 (m, |
| | | 1H), 7.32 (d, J = 7.6 Hz, 1H), 7.12 (s, 1H), 7.01 (br s, 2H), 6.99 (s, 1H), 6.89 (d, |
| | | J = 6.0 Hz, 2H), 6.47 (br s, 1H), 6.15-6.09 (m, 1H), 4.65-4.58 (m, 4H), 4.39- |
| | | 4.29 (m, 2H), 3.80 (dd, J = 4.8, 11.6 Hz, 1H), 3.75-3.71 (m, 3H), 3.67-3.57 |
| | | (m, 3H), 3.30-3.23 (m, 3H), 3.22-3.14 (m, 2H), 3.14-2.98 (m, 6H), 2.84 (br |
| | | t, J = 9.2 Hz, 2H), 2.69-2.61 (m, 1H), 2.27 (br s, 2H), 2.23-2.13 (m, 1H), 1.98 |
| | | (br s, 3H), 1.77-1.68 (m, 2H) |
| I-912 | 730.4 | 12.14 (s, 1H), 10.83 (s, 1H), 8.02-7.74 (m, 1H), 7.32-7.23 (m, 4H), 7.22-7.17 |
| | | (m, 1H), 6.96-6.78 (m, 1H), 6.41-6.36 (m, 2H), 6.19-6.13 (m, 1H), 4.36- |
| | | 4.32 (m, 2H), 4.19-4.10 (m, 3H), 3.64-3.58 (m, 2H), 3.18-2.99 (m, 6H), 2.92- |
| | | 2.83 (m, 3H), 2.78-2.70 (m, 1H), 2.65-2.56 (m, 2H), 2.45-2.35 (m, 1H), |
| | | 2.39-2.34 (m, 1H), 2.27 (d, J = 4.4, Hz, 2H), 2.19-2.16 (m, 3H), 2.14-2.10 |
| | | (m, 1H), 2.08 (d, J = 4.8 Hz, 3H), 1.99 (s, 2H) |
| I-915 | 770.3 | 11.60 (d, J = 5.6 Hz, 1H), 10.88 (s, 1H), 8.16 (s, 1H), 7.74-7.64 (m, 1H), 7.43- |
| | | 7.36 (m, 2H), 7.28 (d, J = 7.6 Hz, 1H), 7.12 (d, J = 2.4 Hz, 2H), 7.08 (s, 1H), |
| | | 7.04-7.01 (m, 1H), 6.93-6.85 (m, 1H), 6.20 (s, 1H), 6.14-6.07 (m, 1H), 4.67- |
| | | 4.60 (m, 2H), 4.40-4.33 (m, 3H), 4.33 (s, 3H), 3.77-3.73 (m, 3H), 3.68 (t, J = |
| | | 5.6 Hz, 1H), 3.60 (t, J = 5.6 Hz, 1H), 3.36 (d, J = 10.0 Hz, 2H), 3.13-3.08 (m, |
| | | 2H), 2.94-2.84 (m, 2H), 2.84-2.77 (m, 1H), 2.70-2.60 (m, 2H), 2.39-2.33 |
| | | (m, 2H), 2.32-2.25 (m, 1H), 2.23-2.16 (m, 1H), 2.04 (s, 4H) |
| I-916 | 800.1 | 11.59 (s, 1H), 11.17-10.98 (m, 1H), 8.09-8.03 (m, 1H), 7.72-7.65 (m, 1H), |
| | | 7.40-7.34 (m, 1H), 7.31-7.25 (m, 1H), 7.09-7.05 (m, 1H), 7.04-6.97 (m, |
| | | 3H), 6.91-6.82 (m, 2H), 6.20 (s, 1H), 6.07 (d, J = 1.2 Hz, 1H), 5.36 (dd, J = |
| | | 5.6, 12.4 Hz, 1H), 4.64-4.56 (m, 1H), 4.45-4.39 (m, 1H), 4.39-4.27 (m, 2H), |
| | | 3.74 (s, 3H), 3.70 (s, 3H), 3.66-3.50 (m, 3H), 3.28 (s, 2H), 2.94-2.85 (m, 3H), |
| | | 2.74 (dd, J = 4.8, 13.2 Hz, 1H), 2.70-2.59 (m, 2H), 2.31 (dd, J = 2.8, 8.4 Hz, |
| | | 1H), 2.26-2.21 (m, 1H), 2.04-1.94 (m, 5H), 1.09-1.04 (m, 2H), 1.02 (d, J = |
| | | 6.8 Hz, 1H) |
| I-917 | 800.4 | 11.59 (s, 1H), 11.17-11.00 (m, 1H), 8.10-8.01 (m, 1H), 7.74-7.62 (m, 1H), |
| | | 7.37 (t, J = 2.8 Hz, 1H), 7.32-7.24 (m, 1H), 7.07 (s, 1H), 7.05-6.97 (m, 3H), |
| | | 6.92-6.81 (m, 2H), 6.20 (d, J = 2.0 Hz, 1H), 6.06 (s, 1H), 5.37 (dd, J = 5.6, 12.4 |
| | | Hz, 1H), 4.65-4.55 (m, 1H), 4.46-4.39 (m, 1H), 4.39 (s, 2H), 3.74 (s, 3H), |
| | | 3.70 (s, 3H), 3.67-3.50 (m, 3H), 3.27 (s, 1H), 3.25 (s, 1H), 2.95-2.85 (m, 3H), |
| | | 2.80-2.74 (m, 1H), 2.71 (dd, J = 4.4, 12.8 Hz, 1H), 2.66-2.60 (m, 1H), 2.35- |
| | | 2.21 (m, 2H), 2.04-1.94 (m, 5H), 1.08-1.01 (m, 3H) |
| I-918 | 773.0 | 12.02 (s, 1H), 10.92 (s, 1H), 7.38-7.29 (m, 1H), 7.16-7.09 (m, 2H), 7.07-6.93 |
| | | (m, 2H), 6.86 (d, J = 14.8 Hz, 1H), 6.55 (d, J = 2.4 Hz, 1H), 6.24-6.14 (m, 1H), |
| | | 5.54-5.42 (m, 1H), 4.53-4.45 (m, 1H), 4.42-4.36 (m, 2H), 3.81 (s, 3H), 3.70- |
| | | 3.64 (m, 2H), 3.29-3.07 (m, 6H), 2.90-2.75 (m, 4H), 2.64 (d, J = 2.8 Hz, |
| | | 1H), 2.55-2.54 (m, 1H), 2.39 (s, 2H), 2.33 (s, 1H), 2.18-2.15 (m, 1H), 2.14 (d, |
| | | J = 7.2 Hz, 3H), 2.13-2.06 (m, 1H), 1.98 (s, 4H) |
| I-921 | 789.6 | 12.42-12.08 (m, 1H), 11.22-10.97 (m, 1H), 8.42 (s, 1H), 7.45-7.24 (m, 1H), |
| | | 7.07-6.84 (m, 4H), 6.45 (d, J = 2.8 Hz, 1H), 6.23-6.11 (m, 1H), 5.45-5.28 |
| | | (m, 1H), 4.48 (s, 2H), 3.72-3.63 (m, 5H), 3.27 (br d, J = 11.2 Hz, 3H), 3.20- |
| | | 3.05 (m, 5H), 2.98-2.88 (m, 4H), 2.75-2.61 (m, 2H), 2.38 (br d, J = 3.6 Hz, |
| | | 1H), 2.20 (br s, 3H), 2.12-2.00 (m, 5H), 1.06-0.98 (m, 6H) |
| I-922 | 758.3 | 12.25-12.14 (m, 1H), 11.11-10.97 (m, 1H), 7.65 (d, J = 8.0 Hz, 2H), 7.48 (d, |
| | | J = 8.0 Hz, 2H), 7.31-7.23 (m, 1H), 7.21-7.16 (m, 1H), 7.09-7.00 (m, 1H), |
| | | 6.90 (d, J = 3.2 Hz, 1H), 6.69 (d, J = 7.2 Hz, 1H), 6.18 (br s, 1H), 5.70 (dd, J = |
| | | 4.8, 11.7 Hz, 1H), 4.45-4.36 (m, 2H), 3.68 (br d, J = 5.6 Hz, 2H), 3.48 (br d, J = |
| | | 8.8 Hz, 2H), 3.27-3.02 (m, 6H), 3.00-2.94 (m, 1H), 2.91-2.78 (m, 4H), |
| | | 2.75-2.71 (m, 1H), 2.71-2.64 (m, 4H), 2.39 (br s, 1H), 2.31-2.16 (m, 2H), |
| | | 2.06-1.96 (m, 4H), 1.08-1.00 (m, 6H) |
| I-923 | 758.3 | 12.20 (s, 1H), 10.88 (s, 1H), 7.64 (d, J = 8.1 Hz, 2H), 7.53-7.45 (m, 2H), 7.40 |
| | | (d, J = 7.6 Hz, 1H), 7.12-7.00 (m, 3H), 6.92-6.85 (m, 1H), 6.20-6.14 (m, |
| | | 1H), 4.44-4.34 (m, 3H), 4.34-4.30 (m, 3H), 3.68 (d, J = 5.6 Hz, 2H), 3.36 (d, |
| | | J = 9.6 Hz, 2H), 3.24-3.00 (m, 6H), 2.99-2.87 (m, 3H), 2.84-2.75 (m, 1H), |
| | | 2.68-2.57 (m, 2H), 2.42-2.33 (m, 2H), 2.32 (s, 1H), 2.22-2.13 (m, 1H), 2.07- |
| | | 1.96 (m, 4H), 1.08-0.99 (m, 6H) |
| I-924 | 772.4 | 12.32-11.94 (m, 1H), 10.88 (s, 1H), 7.40 (d, J = 7.6 Hz, 1H), 7.32 (s, 1H), 7.28 |
| | | (s, 2H), 7.12-7.03 (m, 2H), 6.91-6.81 (m, 1H), 6.40 (d, J = 2.8 Hz, 1H), 6.16 |
| | | (s, 1H), 4.44-4.34 (m, 3H), 4.32 (s, 3H), 3.70-3.62 (m, 2H), 3.41-3.35 (m, |
| | | 2H), 3.23-2.99 (m, 6H), 2.98-2.94 (m, 1H), 2.93-2.85 (m, 2H), 2.80-2.72 |
| | | (m, 1H), 2.70-2.59 (m, 2H), 2.36 (s, 1H), 2.36-2.33 (m, 1H), 2.32-2.25 (m, |
| | | 1H), 2.25-2.20 (m, 1H), 2.16 (s, 3H), 2.04 (d, J = 3.2 Hz, 4H), 1.07-0.99 (m, |
| | | 6H) |
| I-925 | 826.1 | 12.33-12.09 (m, 1H), 10.84-10.71 (m, 1H), 7.83-7.58 (m, 1H), 7.48-7.28 |
| | | (m, 2H), 7.04-6.89 (m, 3H), 6.69-6.62 (m, 1H), 6.59-6.51 (m, 1H), 6.49- |
| | | 6.43 (m, 1H), 6.22-6.11 (m, 2H), 5.90-5.80 (m, 1H), 4.37-4.35 (m, 2H), 4.32- |
| | | 4.26 (m, 2H), 3.58 (br s, 2H), 3.27 (br s, 6H), 3.19-3.15 (m, 2H), 3.09-3.03 |
| | | (m, 6H), 3.00-2.96 (m, 2H), 2.80-2.70 (m, 1H), 2.62-2.55 (m, 1H), 2.60- |
| | | 2.54 (m, 1H), 2.35-2.30 (m, 2H), 2.29-2.20 (m, 1H), 2.15-2.06 (m, 1H), 1.92- |
| | | 1.81 (m, 1H). |
| I-926 | 822.1 | 12.44-11.93 (m, 1H), 10.96 (d, J = 9.6 Hz, 1H), 7.76-7.67 (m, 1H), 7.47-7.33 |
| | | (m, 1H), 7.12-7.05 (m, 1H), 7.05-6.98 (m, 1H), 6.93 (t, J = 9.2 Hz, 1H), 6.90- |
| | | 6.80 (m, 1H), 6.60-6.51 (m, 1H), 6.50-6.41 (m, 2H), 6.23-6.10 (m, 2H), |
| | | 5.86 (d, J = 8.0 Hz, 1H), 4.39-4.33 (m, 3H), 4.33-4.22 (m, 2H), 3.68-3.60 |
| | | (m, 1H), 3.58 (t, J = 5.6 Hz, 1H), 3.21 (s, 6H), 3.06 (s, 6H), 3.01-2.94 (m, 3H), |
| | | 2.80-2.71 (m, 1H), 2.60 (d, J = 4.0 Hz, 1H), 2.33 (d, J = 1.2 Hz, 2H), 2.29- |
| | | 2.22 (m, 1H), 2.15-2.08 (m, 1H), 2.05 (s, 3H), 1.93-1.81 (m, 1H) |
| I-932 | 830.3 | 11.20-10.95 (m, 1H), 8.26-8.18 (m, 1H), 7.71-7.59 (m, 1H), 7.54-7.49 (m, |
| | | 1H), 7.49-7.41 (m, 2H), 7.05-6.97 (m, 2H), 6.97-6.87 (m, 2H), 5.37 (br dd, |
| | | J = 5.6, 12.4 Hz, 1H), 4.39 (br s, 2H), 3.70 (s, 3H), 3.63-3.54 (m, 2H), 3.26 (br |
| | | d, J = 11.6 Hz, 2H), 3.00 (br d, J = 9.6 Hz, 6H), 2.95-2.83 (m, 4H), 2.73-2.60 |
| | | (m, 2H), 2.55-2.53 (m, 1H), 2.45 (br d, J = 2.0 Hz, 2H), 2.39-2.30 (m, 1H), |
| | | 2.08-2.05 (m, 2H), 2.02 (br d, J = 7.6 Hz, 4H) |
| I-933 | 816.4 | 12.15-11.99 (m, 1H), 10.41-10.23 (m, 1H), 7.76-7.69 (m, 1H), 7.49 (d, J = |
| | | 8.4 Hz, 2H), 7.45-7.36 (m, 1H), 7.14-7.09 (m, 1H), 7.01-6.97 (m, 1H), 6.92 |
| | | (dd, J = 6.0, 16.4 Hz, 1H), 6.85 (s, 1H), 6.76-6.71 (m, 1H), 6.70-6.64 (m, 2H), |
| | | 6.23-6.17 (m, 1H), 6.15-6.11 (m, 1H), 4.37 (q, J = 7.2 Hz, 4H), 4.29 (s, 1H), |
| | | 3.70 (s, 2H), 3.64 (d, J = 6.0 Hz, 2H), 3.58 (s, 2H), 3.52-3.51 (m, 2H), 3.43 (s, |
| | | 6H), 3.00-2.96 (m, 2H), 2.69 (t, J = 6.4 Hz, 4H), 2.34 (s, 2H), 2.27 (s, 1H), |
| | | 2.09-1.97 (m, 4H) |
| I-934 | 783.4 | 12.27-12.12 (m, 1H), 10.62-10.42 (m, 1H), 8.61-8.38 (m, 1H), 7.73-7.65 |
| | | (m, 3H), 7.61-7.53 (m, 2H), 7.43-7.37 (m, 1H), 7.22-7.17 (m, 1H), 7.10- |
| | | 7.00 (m, 2H), 6.92-6.83 (m, 2H), 6.23-6.12 (m, 2H), 4.36 (br d, J = 7.2 Hz, |
| | | 4H), 4.34 (br s, 2H), 4.25 (s, 3H), 4.08-4.01 (m, 1H), 3.99-3.92 (m, 2H), 3.89 |
| | | (t, J = 6.8 Hz, 2H), 3.68-3.62 (m, 1H), 3.58 (br t, J = 5.6 Hz, 1H), 3.22-3.02 |
| | | (m, 6H), 3.00-2.95 (m, 2H), 2.78-2.73 (m, 2H), 2.35-2.25 (m, 2H) |
| I-935 | 759.3 | 12.24-12.09 (m, 1H), 11.21-10.92 (m, 1H), 7.42 (s, 1H), 7.34 (s, 2H), 6.97 (s, |
| | | 1H), 6.97-6.85 (m, 1H), 6.84-6.73 (m, 1H), 6.50-6.41 (m, 1H), 6.34-6.24 |
| | | (m, 1H), 6.22-6.14 (m, 1H), 5.40-5.27 (m, 1H), 4.42-4.34 (m, 2H), 3.94- |
| | | 3.85 (m, 1H), 3.65-3.62 (m, 1H), 3.60 (s, 3H), 3.19-3.01 (m, 6H), 2.91-2.85 |
| | | (m, 1H), 2.72-2.60 (m, 2H), 2.38 (br s, 2H), 2.30-2.24 (m, 1H), 2.23-2.18 |
| | | (m, 3H), 2.08 (d, J = 3.2 Hz, 3H), 2.04-1.90 (m, 8H), 1.71-1.61 (m, 2H). |
| I-936 | 804.2 | δ (ppm) = 11.8-11.69 (s, 1H), 11.13-11.06 (s, 1H), 8.16-8.05 (m, 1H), 7.74- |
| | | 7.64 (m, 1H), 7.46-7.40 (d, J = 2.4 Hz, 1H), 7.26-7.18 (m, 2H), 7.02-6.98 |
| | | (m, 2H), 6.97-6.87 (m, 2H), 6.33-6.25 (s, 1H), 6.18-6.07 (m, 1H), 5.42- |
| | | 5.30 (m, 1H), 4.65-4.59 (m, 2H), 4.40-4.30 (m, 2H), 3.73-3.67 (s, 3H), 3.67- |
| | | 3.59 (m, 2H), 3.58-3.55 (d, J = 3.6 Hz, 3H), 3.250-3.22 (d, J = 10.6 Hz, |
| | | 3H), 3.12-3.07 (m, 2H), 3.06-3.00 (m, 1H), 2.96-2.86 (m, 3H), 2.73-2.60 |
| | | (m, 2H), 2.37-2.26 (m, 2H), 2.03-1.98 (m, 2H), 1.96-1.92 (m, 2H) |
| I-937 | 804.4 | 11.76 (d, J = 5.6 Hz, 1H), 11.27-11.08 (m, 1H), 8.25-8.13 (m, 1H), 7.84-7.69 |
| | | (m, 1H), 7.48 (d, J = 2.4 Hz, 1H), 7.24-7.15 (m, 2H), 7.13-7.06 (m, 2H), 7.04- |
| | | 6.95 (m, 2H), 6.32 (s, 1H), 6.20 (d, J = 11.1 Hz, 1H), 5.50-5.40 (m, 1H), 4.75- |
| | | 4.67 (m, 2H), 4.48-4.37 (m, 2H), 3.84 (d, J = 2.4 Hz, 3H), 3.80 (s, 3H), 3.75- |
| | | 3.67 (m, 2H), 3.36 (d, J = 11.2 Hz, 2H), 3.21-3.15 (m, 2H), 3.13-3.07 (m, |
| | | 1H), 3.04-2.93 (m, 3H), 2.85-2.76 (m, 1H), 2.74-2.68 (m, 1H), 2.45-2.41 |
| | | (m, 1H), 2.37-2.36 (m, 1H), 2.36 (s, 1H), 2.22-2.08 (m, 3H), 2.04 (d, J = 11.2 |
| | | Hz, 2H) |
| I-938 | 887.7 | 12.01 (d, J = 2.8 Hz, 1H), 11.09 (s, 1H), 7.26 (d, J = 7.2 Hz, 1H), 7.02 (s, 1H), |
| | | 6.97 (d, J = 8.0 Hz, 1H), 6.95-6.86 (m, 4H), 6.48 (s, 1H), 6.11 (s, 1H), 5.35 |
| | | (dd, J = 5.6, 11.9 Hz, 1H), 4.36 (d, J = 9.2 Hz, 2H), 3.73 (s, 3H), 3.68-3.62 (m, |
| | | 5H), 3.21-3.14 (m, 6H), 3.10-3.05 (m, 4H), 2.94-2.88 (m, 2H), 2.78-2.69 |
| | | (m, 3H), 2.60-2.57 (m, 1H), 2.38-2.30 (m, 5H), 2.06-1.95 (m, 2H), 1.92- |
| | | 1.84 (m, 4H), 1.78-1.68 (m, 4H), 1.06-0.98 (m, 6H) |
| I-945 | 837.3 | 12.17-12.07 (m, 1H), 10.91-10.81 (m, 1H), 7.75-7.68 (m, 1H), 7.46-7.34 |
| | | (m, 2H), 7.00-6.85 (m, 3H), 6.74 (d, J = 6.8 Hz, 1H), 6.62 (s, 1H), 6.51 (d, J = |
| | | 12.8 Hz, 1H), 6.22-6.16 (m, 1H), 6.16-6.09 (m, 1H), 5.29 (d, J = 6.8 Hz, 1H), |
| | | 4.41-4.25 (m, 5H), 3.94 (d, J = 12.4 Hz, 2H), 3.80 (s, 3H), 3.67-3.55 (m, 2H), |
| | | 3.26-3.00 (m, 6H), 3.00-2.94 (m, 2H), 2.93-2.77 (m, 4H), 2.56 (d, J = 2.8 |
| | | Hz, 1H), 2.33 (d, J = 1.6 Hz, 1H), 2.29-2.22 (m, 1H), 2.18-2.08 (m, 1H), 2.01- |
| | | 1.76 (m, 5H) |
| I-946 | 810.3 | 12.17 (s, 1H), 10.89 (m, 1H), 7.73 (d, J = 9.6 Hz, 1H), 7.68-7.61 (m, 2H), 7.52- |
| | | 7.46 (m, 2H), 7.45-7.35 (m, 2H), 7.12-7.00 (m, 3H), 6.91-6.87 (m, 1H), |
| | | 6.23-6.11 (m, 2H), 4.35 (s, 4H), 4.34-4.32 (m, 3H), 3.68-3.58 (m, 2H), 3.39 |
| | | (s, 1H), 3.27-3.05 (m, 6H), 3.01-2.97 (m, 2H), 2.93-2.79 (m, 3H), 2.69-2.63 |
| | | (m, 2H), 2.28 (s, 4H), 2.22-2.15 (m, 1H), 2.09-2.07 (m, 1H), 2.02 (s, 4H). |
| I-947 | 836.4 | 0.58-0.80 (m, 4 H) 2.01 (br d, J = 2.4 Hz, 4 H) 2.18 (br dd, J = 14.0, 5.2 Hz, 1 |
| | | H) 2.24-2.39 (m, 4 H) 2.62-2.69 (m, 2 H) 2.80-2.93 (m, 3 H) 2.94-3.00 (m, |
| | | 2 H) 3.05 (s, 3 H) 3.15 (br s, 1 H) 3.54-3.69 (m, 2 H) 4.23-4.43 (m, 9 H) 6.11- |
| | | 6.25 (m, 2 H) 6.99-7.11 (m, 4 H) 7.37-7.44 (m, 2 H) 7.48 (d, J = 8.0 Hz, 2 |
| | | H) 7.64 (br d, J = 8.0 Hz, 2 H) 7.68-7.75 (m, 1 H) 10.88 (s, 1 H) 12.03-12.15 |
| | | (m, 1 H). |
| I-950 | 852.3 | 12.24-12.07 (m, 1H), 10.86 (br s, 1H), 7.79-7.66 (m, 1H), 7.49-7.34 (m, 1H), |
| | | 7.13-6.97 (m, 2H), 6.91-6.79 (m, 1H), 6.71 (d, J = 8.0 Hz, 1H), 6.59 (d, J = |
| | | 14.4 Hz, 1H), 6.45 (d, J = 2.4 Hz, 1H), 6.25-6.10 (m, 2H), 5.14 (d, J = 6.8 Hz, |
| | | 1H), 4.40-4.26 (m, 5H), 3.83 (s, 3H), 3.68-3.54 (m, 2H), 3.21 (br d, J = 3.6 |
| | | Hz, 4H), 3.18-3.03 (m, 9H), 2.98 (q, J = 6.8 Hz, 3H), 2.82 (ddd, J = 5.2, 13.2, |
| | | 17.6 Hz, 1H), 2.57 (br d, J = 3.2 Hz, 1H), 2.36-2.23 (m, 2H), 2.20-2.12 (m, |
| | | 1H), 2.06 (br s, 3H), 2.01-1.86 (m, 1H) |
| I-974 | 734.1 | 13.83-13.42 (m, 1H), 11.22-10.90 (m, 1H), 7.86 (br s, 1H), 7.36 (br d, J = 7.6 |
| | | Hz, 1H), 7.12 (s, 1H), 7.09-6.95 (m, 4H), 6.91 (br d, J = 7.2 Hz, 1H), 6.20 (br |
| | | s, 1H), 5.37 (br dd, J = 5.2, 12.8 Hz, 1H), 4.51-4.31 (m, 2H), 3.79 (s, 3H), 3.74- |
| | | 3.64 (m, 5H), 3.27 (br d, J = 11.2 Hz, 2H), 2.99-2.85 (m, 4H), 2.80-2.60 (m, |
| | | 3H), 2.39 (br s, 1H), 2.34-2.20 (m, 1H), 2.04-1.95 (m, 5H), 1.07-0.99 (m, |
| | | 6H) |
| I-992 | 748.4 | 12.28-11.92 (m, 1H), 11.38-10.52 (m, 1H), 7.62 (br d, J = 7.6 Hz, 2H), 7.46 |
| | | (br d, J = 7.2 Hz, 2H), 7.12-7.04 (m, 1H), 7.04-6.94 (m, 2H), 6.92-6.83 (m, |
| | | 2H), 5.41-5.31 (m, 1H), 4.53-4.42 (m, 1H), 3.93-3.82 (m, 1H), 3.70 (s, 3H), |
| | | 3.24 (br s, 6H), 3.12 (br s, 2H), 3.03-2.98 (m, 1H), 2.90 (br s, 2H), 2.81-2.76 |
| | | (m, 1H), 2.76-2.67 (m, 2H), 2.64 (br d, J = 4.4 Hz, 1H), 2.61 (br s, 1H), 2.04 |
| | | (br d, J = 10.4 Hz, 3H), 1.96 (br s, 6H), 1.77 (br d, J = 15.2 Hz, 1H), 1.67-1.34 |
| | | (m, 2H), 1.29-1.06 (m, 1H) |
| I-989 | 774.4 | 12.49-11.89 (m, 1H), 11.30-10.58 (m, 1H), 7.64 (d, J = 8.0 Hz, 2H), 7.45 (d, |
| | | J = 8.0 Hz, 2H), 7.07-6.96 (m, 3H), 6.92-6.86 (m, 2H), 6.16 (s, 1H), 5.43- |
| | | 5.31 (m, 1H), 4.43-4.34 (m, 2H), 3.73-3.65 (m, 5H), 3.28-3.17 (m, 5H), 3.14- |
| | | 3.03 (m, 3H), 2.98-2.94 (m, 1H), 2.93-2.85 (m, 3H), 2.69 (d, J = 4.4 Hz, |
| | | 2H), 2.66-2.59 (m, 1H), 2.39-2.23 (m, 2H), 2.07-1.92 (m, 5H), 1.06-0.99 |
| | | (m, 6H) |
| I-988 | 837.5 | 12.20 (d, J = 7.2 Hz, 1H), 10.87 (s, 1H), 7.72 (dd, J = 2.0, 10.4 Hz, 1H), 7.50- |
| | | 7.35 (m, 3H), 7.19 (t, J = 8.8 Hz, 1H), 7.08-6.98 (m, 1H), 6.91-6.77 (m, 2H), |
| | | 6.52 (d, J = 12.8 Hz, 1H), 6.24-6.13 (m, 2H), 5.30 (d, J = 6.4 Hz, 1H), 4.40- |
| | | 4.27 (m, 5H), 3.82 (s, 3H), 3.67-3.51 (m, 4H), 3.23-3.05 (m, 5H), 3.03-2.93 |
| | | (m, 3H), 2.88-2.77 (m, 4H), 2.57 (d, J = 2.8 Hz, 1H), 2.33 (d, J = 1.6 Hz, 1H), |
| | | 2.27 (d, J = 2.4 Hz, 1H), 2.17-2.10 (m, 1H), 2.01-1.89 (m, 3H), 1.81 (d, J = |
| | | 11.4 Hz, 2H) |
| I-987 | 837.6 | 10.98-10.77 (m, 1H), 8.11 (d, J = 11.8 Hz, 1H), 7.74-7.65 (m, 2H), 7.49 (d, J = |
| | | 8.8 Hz, 2H), 7.41-7.31 (m, 1H), 7.12 (br d, J = 8.8 Hz, 2H), 6.76-6.68 (m, |
| | | 1H), 6.52 (d, J = 12.8 Hz, 1H), 6.31-6.25 (m, 1H), 5.30 (br d, J = 6.0 Hz, 1H), |
| | | 4.67-4.59 (m, 2H), 4.41-4.28 (m, 3H), 3.96-3.89 (m, 2H), 3.81 (s, 3H), 3.69- |
| | | 3.59 (m, 2H), 3.18-3.00 (m, 9H), 2.89-2.79 (m, 4H), 2.60-2.55 (m, 1H), |
| | | 2.29 (br d, J = 3.2 Hz, 1H), 2.16-2.11 (m, 1H), 1.89 (br s, 2H), 1.87-1.81 (m, |
| | | 3H) |
| I-986 | 807.7 | 12.12 (d, J = 8.8 Hz, 1H), 10.79 (s, 1H), 7.75-7.68 (m, 1H), 7.46-7.34 (m, |
| | | 2H), 7.02 (t, J = 8.4 Hz, 1H), 6.98-6.86 (m, 3H), 6.62 (s, 1H), 6.51-6.43 (m, |
| | | 2H), 6.22-6.16 (m, 1H), 6.15-6.09 (m, 1H), 6.03 (d, J = 7.6 Hz, 1H), 4.40- |
| | | 4.27 (m, 5H), 3.93 (d, J = 12.0 Hz, 2H), 3.67-3.56 (m, 2H), 3.26-2.91 (m, 9H), |
| | | 2.86 (t, J = 10.4 Hz, 3H), 2.79-2.68 (m, 1H), 2.61-2.54 (m, 1H), 2.35-2.24 |
| | | (m, 2H), 2.13-2.05 (m, 1H), 1.91-1.70 (m, 5H) |
| I-985 | 786.4 | 11.97-11.90 (m, 1H), 10.96-10.84 (m, 1H), 7.42-7.38 (m, 1H), 7.33-7.27 |
| | | (m, 1H), 7.10-7.02 (m, 4H), 6.95-6.87 (m, 1H), 6.75-6.68 (m, 1H), 6.17- |
| | | 6.08 (m, 1H), 4.37-4.30 (m, 6H), 3.77-3.73 (m, 3H), 3.64-3.57 (m, 2H), 3.41- |
| | | 3.36 (m, 2H), 3.11-3.06 (m, 1H), 3.04-3.01 (m, 3H), 2.91-2.78 (m, 3H), |
| | | 2.69-2.62 (m, 2H), 2.40-2.31 (m, 3H), 2.29-2.24 (m, 1H), 2.21-2.15 (m, |
| | | 1H), 2.08 (d, J = 6.4 Hz, 3H), 2.05-2.01 (m, 3H), 0.76-0.70 (m, 2H), 0.65- |
| | | 0.58 (m, 2H) |
| I-982 | 802.3 | 11.97-11.89 (m, 1H), 11.15-11.05 (m, 1H), 7.36-7.25 (m, 1H), 7.09-7.06 |
| | | (m, 1H), 7.04-6.96 (m, 3H), 6.95-6.87 (m, 2H), 6.75-6.67 (m, 1H), 6.17- |
| | | 6.07 (m, 1H), 5.41-5.33 (m, 1H), 4.37-4.30 (m, 2H), 3.77-3.73 (m, 3H), 3.70 |
| | | (s, 3H), 3.64-3.58 (m, 2H), 3.28-3.24 (m, 2H), 3.11-3.06 (m, 1H), 3.05-3.01 |
| | | (m, 3H), 2.93-2.85 (m, 3H), 2.68 (br s, 2H), 2.66-2.59 (m, 1H), 2.37 (br s, |
| | | 1H), 2.31-2.23 (m, 1H), 2.10-2.06 (m, 3H), 2.03-1.94 (m, 5H), 0.76-0.69 |
| | | (m, 2H), 0.63-0.56 (m, 2H). |
| I-976 | 808.5 | 12.20-12.10 (m, 1H), 10.80 (br s, 1H), 7.77-7.68 (m, 1H), 7.47-7.35 (m, 2H), |
| | | 6.98-6.88 (m, 4H), 6.63-6.53 (m, 2H), 6.46 (br d, J = 8.4 Hz, 1H), 6.22-6.17 |
| | | (m, 1H), 6.12 (br s, 1H), 5.87 (br d, J = 7.6 Hz, 1H), 4.36 (br d, J = 6.4 Hz, 3H), |
| | | 4.32-4.25 (m, 2H), 3.66-3.63 (m, 1H), 3.58 (br t, J = 5.6 Hz, 2H), 3.49 (br s, |
| | | 2H), 3.33-3.29 (m, 2H), 3.22-3.12 (m, 4H), 3.01 (br d, J = 5.6 Hz, 4H), 2.99- |
| | | 2.95 (m, 2H), 2.78-2.71 (m, 1H), 2.60 (br d, J = 4.4 Hz, 1H), 2.55 (br d, J = |
| | | 4.8 Hz, 1H), 2.35-2.25 (m, 2H), 2.10 (td, J = 4.0, 8.4 Hz, 1H), 1.92-1.81 (m, |
| | | 1H) |
| I-975 | 838.2 | 13.92-13.58 (m, 1H), 11.09 (br s, 1H), 8.11 (d, J = 10.4 Hz, 1H), 7.90 (br s, |
| | | 1H), 7.72-7.66 (m, 1H), 7.36 (dd, J = 6.8, 13.1 Hz, 1H), 7.21 (d, J = 8.1 Hz, |
| | | 1H), 7.09-6.92 (m, 4H), 6.23 (br d, J = 8.8 Hz, 1H), 5.38 (dd, J = 5.2, 12.8 Hz, |
| | | 1H), 4.63 (q, J = 7.2 Hz, 2H), 4.42-4.30 (m, 2H), 3.70-3.58 (m, 5H), 3.20- |
| | | 3.07 (m, 8H), 2.97-2.84 (m, 1H), 2.76-2.61 (m, 8H), 2.41-2.25 (m, 2H), 2.07- |
| | | 1.97 (m, 1H) |
| I-973 | 840.3 | 12.13-12.03 (m, 1H), 11.21-11.03 (m, 1H), 7.76-7.68 (m, 1H), 7.55 (d, J = |
| | | 8.4 Hz, 2H), 7.47-7.38 (m, 1H), 7.13 (d, J = 8.4 Hz, 2H), 7.07-6.99 (m, 4H), |
| | | 6.85 (s, 1H), 6.25-6.15 (m, 1H), 5.40 (dd, J = 5.6, 12.8 Hz, 1H), 4.49 (d, J = |
| | | 2.8 Hz, 3H), 4.36 (q, J = 7.6 Hz, 2H), 4.15-4.06 (m, 2H), 3.98-3.88 (m, 3H), |
| | | 3.66 (s, 3H), 3.53-3.43 (m, 2H), 3.10-3.03 (m, 1H), 2.97-2.90 (m, 4H), 2.83- |
| | | 2.59 (m, 4H), 2.33 (t, J = 8.0 Hz, 2H), 2.06-2.00 (m, 1H), 1.99-1.87 (m, 6H), |
| | | 1.81-1.74 (m, 1H), 1.59-1.40 (m, 1H) |
| I-970 | 834.4 | 12.12 (br d, J = 7.6 Hz, 1H), 10.86 (s, 1H), 7.78-7.67 (m, 1H), 7.52-7.36 (m, |
| | | 2H), 6.88-6.76 (m, 2H), 6.73 (br d, J = 7.2 Hz, 1H), 6.52 (d, J = 12.8 Hz, 1H), |
| | | 6.46 (br s, 1H), 6.24-6.17 (m, 1H), 6.16-6.11 (m, 1H), 5.29 (d, J = 6.8 Hz, |
| | | 1H), 4.55 (br d, J = 12.8 Hz, 2H), 4.40-4.35 (m, 3H), 4.34 (br d, J = 3.2 Hz, |
| | | 1H), 4.30 (br s, 1H), 3.80 (s, 3H), 3.66-3.57 (m, 2H), 3.23-3.01 (m, 6H), 2.98 |
| | | (q, J = 6.8 Hz, 3H), 2.92 (br s, 1H), 2.89 (br s, 1H), 2.86 (br d, J = 4.0 Hz, 1H), |
| | | 2.84-2.74 (m, 1H), 2.33 (br d, J = 2.0 Hz, 1H), 2.26 (br s, 1H), 2.23 (d, J = 2.4 |
| | | Hz, 3H), 2.18-2.11 (m, 1H), 2.01-1.90 (m, 1H), 1.84-1.71 (m, 4H) |
| I-969 | 865.4 | 12.27-12.04 (m, 1H), 10.99-10.76 (m, 1H), 7.82-7.63 (m, 1H), 7.49-7.32 |
| | | (m, 1H), 7.06-6.98 (m, 2H), 6.80 (d, J = 6.4 Hz, 2H), 6.57-6.49 (m, 1H), 6.39 |
| | | (br d, J = 0.9 Hz, 1H), 6.21-6.12 (m, 2H), 5.34-5.28 (m, 1H), 4.42-4.28 (m, |
| | | 6H), 3.83 (s, 3H), 3.60-3.51 (m, 4H), 3.19-3.06 (m, 6H), 3.00-2.95 (m, 3H), |
| | | 2.88-2.81 (m, 4H), 2.33 (br s, 2H), 2.26 (br s, 1H), 2.17-2.11 (m, 1H), 1.99- |
| | | 1.91 (m, 3H), 1.85-1.79 (m, 2H), 0.99-0.94 (m, 3H) |
| I-967 | 807.4 | 12.19 (d, J = 5.6 Hz, 1H), 10.78 (s, 1H), 7.86 (dd, J = 4.4, 12.4 Hz, 1H), 7.61 (br |
| | | d, J = 8.0 Hz, 2H), 7.47-7.37 (m, 3H), 7.09-6.99 (m, 1H), 6.94-6.85 (m, 2H), |
| | | 6.53 (dd, J = 2.4, 15.2 Hz, 1H), 6.45 (dd, J = 2.0, 8.8 Hz, 1H), 6.16 (br s, 1H), |
| | | 5.82 (d, J = 7.6 Hz, 1H), 4.38 (s, 1H), 4.34-4.23 (m, 4H), 3.69-3.55 (m, 2H), |
| | | 3.30-3.02 (m, 8H), 3.00-2.94 (m, 2H), 2.79-2.64 (m, 4H), 2.58 (td, J = 4.0, |
| | | 17.6 Hz, 1H), 2.37-2.24 (m, 2H), 2.09 (dt, J = 4.4, 8.8 Hz, 1H), 1.94-1.79 (m, |
| | | 5H) |
| I-966 | 807.3 | 12.25-12.12 (m, 1H), 10.78 (s, 1H), 7.68-7.56 (m, 3H), 7.42 (d, J = 8.0 Hz, |
| | | 2H), 7.09-6.97 (m, 1H), 6.95-6.83 (m, 2H), 6.57-6.42 (m, 2H), 6.16 (br s, |
| | | 1H), 5.97-5.68 (m, 2H), 4.40-4.30 (m, 2H), 4.29-4.16 (m, 3H), 3.70-3.55 |
| | | (m, 2H), 3.26 (br d, J = 11.2 Hz, 2H), 3.22-3.01 (m, 6H), 2.98-2.90 (m, 2H), |
| | | 2.80-2.65 (m, 4H), 2.61-2.54 (m, 1H), 2.37-2.24 (m, 2H), 2.10 (td, J = 4.4, |
| | | 8.8 Hz, 1H), 1.93-1.80 (m, 5H) |
| I-963 | 771.4 | 13.85-13.70 (m, 1H), 11.11 (s, 1H), 8.14-8.08 (m, 1H), 7.85-7.76 (m, 1H), |
| | | 7.73-7.65 (m, 1H), 7.35-7.32 (m, 1H), 7.28 (s, 2H), 7.02-6.98 (m, 2H), 6.90 |
| | | (d, J = 8.4 Hz, 2H), 6.28-6.20 (m, 1H), 5.41-5.34 (m, 1H), 4.63 (q, J = 7.2 Hz, |
| | | 2H), 4.37 (d, J = 17.2 Hz, 2H), 3.71 (s, 3H), 3.68-3.57 (m, 3H), 3.11 (d, J = 6.4 |
| | | Hz, 2H), 2.94-2.86 (m, 3H), 2.73 (d, J = 4.0 Hz, 1H), 2.62 (s, 1H), 2.39-2.32 |
| | | (m, 2H), 2.32-2.26 (m, 1H), 2.19 (s, 3H), 2.04-1.92 (m, 6H) |
| I-962 | 775.3 | 13.81 (br s, 1H), 11.09 (s, 1H), 8.10 (d, J = 12.4 Hz, 1H), 8.00 (br s, 1H), 7.68 |
| | | (d, J = 12.0 Hz, 1H), 7.59 (dt, J = 2.8, 8.0 Hz, 1H), 7.41-7.30 (m, 2H), 7.17- |
| | | 7.06 (m, 1H), 7.04-6.95 (m, 2H), 6.90 (br d, J = 7.2 Hz, 1H), 6.22 (br s, 1H), |
| | | 5.36 (br dd, J = 5.2, 12.8 Hz, 1H), 4.68-4.57 (m, 2H), 4.37 (br d, J = 17.2 Hz, |
| | | 2H), 3.70 (s, 3H), 3.68-3.59 (m, 2H), 3.25 (br d, J = 11.2 Hz, 2H), 3.15-3.06 |
| | | (m, 2H), 2.96-2.76 (m, 4H), 2.74-2.58 (m, 2H), 2.42-2.25 (m, 2H), 2.07- |
| | | 1.86 (m, 5H). |
| I-961 | 803.4 | 12.24-12.09 (m, 1H), 10.84-10.68 (m, 1H), 7.75-7.68 (m, 1H), 7.62 (br d, J = |
| | | 8.0 Hz, 2H), 7.46-7.36 (m, 3H), 7.07-6.93 (m, 2H), 6.87 (br s, 1H), 6.73- |
| | | 6.64 (m, 1H), 6.56 (br d, J = 7.2 Hz, 1H), 6.23-6.11 (m, 2H), 4.87-4.73 (m, |
| | | 1H), 4.39-4.30 (m, 4H), 3.66 (br s, 1H), 3.58 (br s, 1H), 3.22-3.07 (m, 6H), |
| | | 3.00-2.96 (m, 2H), 2.88-2.81 (m, 1H), 2.75 (br d, J = 2.8 Hz, 2H), 2.70 (s, |
| | | 3H), 2.68-2.66 (m, 1H), 2.56 (br s, 2H), 2.36-2.24 (m, 4H), 1.94-1.84 (m, |
| | | 5H) |
| I-711 | 865.1 | 12.25-12.14 (m, 1H), 11.10 (s, 1H), 7.32 (dd, J = 7.2, 13.2 Hz, 1H), 7.12 (dd, |
| | | J = 7.6, 12.4 Hz, 1H), 7.03-6.94 (m, 3H), 6.93-6.87 (m, 1H), 6.66 (s, 1H), |
| | | 6.14 (s, 1H), 5.37 (dd, J = 5.2, 12.4 Hz, 1H), 4.43-4.32 (m, 2H), 3.69-3.62 |
| | | (m, 3H), 3.59 (s, 3H), 3.36-3.33 (m, 6H), 3.19-3.05 (m, 6H), 3.01-2.91 (m, |
| | | 2H), 2.91-2.84 (m, 1H), 2.76-2.66 (m, 2H), 2.64-2.59 (m, 2H), 2.40-2.29 |
| | | (m, 2H), 2.25 (s, 1H), 2.03-1.96 (m, 1H), 1.06-0.99 (m, 6H), 0.91 (d, J = 4.0 |
| | | Hz, 1H), 0.72 (s, 2H) |
| I-716 | 864.3 | 11.97 (br d, J = 8.0 Hz, 1H), 10.71 (br s, 1H), 7.65-7.55 (m, 1H), 7.34-7.17 |
| | | (m, 2H), 6.84-6.73 (m, 1H), 6.50-6.38 (m, 4H), 6.33 (d, J = 8.4 Hz, 1H), |
| | | 6.11-6.03 (m, 1H), 6.02-5.95 (m, 1H), 4.83 (d, J = 6.2 Hz, 1H), 4.27-4.21 |
| | | (m, 3H), 4.20-4.04 (m, 2H), 3.66 (s, 3H), 3.55-3.38 (m, 4H), 3.29-3.23 (m, |
| | | 2H), 3.18-3.12 (m, 3H), 3.08-2.81 (m, 12H), 2.68 (ddd, J = 5.2, 13.2, 18.0 |
| | | Hz, 1H), 2.26-2.10 (m, 2H), 2.07-1.98 (m, 1H), 1.83-1.70 (m, 1H). |
| I-717 | 834.3 | 13.72 (br s, 1H), 10.85 (s, 1H), 8.25-8.09 (m, 2H), 7.69 (d, J = 12.0 Hz, 1H), |
| | | 7.63-7.57 (m, 2H), 7.12-7.00 (m, 3H), 6.65-6.53 (m, 2H), 6.23 (br s, 1H), |
| | | 5.09 (br d, J = 6.8 Hz, 1H), 4.68-4.59 (m, 2H), 4.38 (br d, J = 17.6 Hz, 2H), |
| | | 4.26 (td, J = 5.6, 11.6 Hz, 1H), 3.80 (s, 3H), 3.67 (br t, J = 5.2 Hz, 1H), 3.62 |
| | | (br t, J = 5.2 Hz, 1H), 3.25 (br s, 6H), 3.14-3.07 (m, 2H), 2.86-2.76 (m, 1H), |
| | | 2.73-2.64 (m, 6H), 2.57 (br s, 1H), 2.37 (br s, 2H), 2.30 (br d, J = 1.6 Hz, |
| | | 1H), 2.19-2.10 (m, 1H), 1.98-1.86 (m, 3H), 1.68-1.56 (m, 2H) |
| I-718 | 704.3 | 14.15-13.50 (m, 1H), 10.81 (br s, 1H), 8.24 (br s, 1H), 8.10 (d, J = 12.4 Hz, |
| | | 1H), 7.72-7.65 (m, 3H), 7.46 (d, J = 8.4 Hz, 2H), 7.18-7.08 (m, 1H), 7.07- |
| | | 7.02 (m, 2H), 7.01-6.96 (m, 1H), 6.24 (br d, J = 4.0 Hz, 1H), 4.66-4.59 (m, |
| | | 2H), 4.38 (br d, J = 17.2 Hz, 2H), 3.82 (dd, J = 4.8, 12.0 Hz, 1H), 3.69-3.60 |
| | | (m, 2H), 3.49 (br d, J = 11.6 Hz, 2H), 3.13-3.07 (m, 2H), 2.86-2.79 (m, 2H), |
| | | 2.77-2.70 (m, 1H), 2.68-2.61 (m, 1H), 2.52 (br s, 1H), 2.37 (br d, J = 1.6 Hz, |
| | | 1H), 2.30 (br s, 1H), 2.25-2.16 (m, 1H), 2.02 (td, J = 4.4, 8.8 Hz, 1H), 1.96- |
| | | 1.86 (m, 4H) |
| I-719 | 735.3 | 13.61 (br s, 1H), 10.82 (s, 1H), 8.10 (d, J = 11.2 Hz, 1H), 7.84 (br s, 1H), 7.68 |
| | | (d, J = 12.0 Hz, 1H), 7.32 (d, J = 7.6 Hz, 1H), 7.15-6.91 (m, 6H), 6.26-6.14 |
| | | (m, 1H), 4.68-4.55 (m, 2H), 4.36 (br d, J = 20.0 Hz, 2H), 3.82 (dd, J = 4.8, |
| | | 11.6 Hz, 1H), 3.77 (d, J = 4.0 Hz, 3H), 3.68-3.58 (m, 2H), 3.50 (br d, J = 11.6 |
| | | Hz, 2H), 3.10 (q, J = 7.2 Hz, 2H), 2.88-2.72 (m, 3H), 2.66 (ddd, J = 5.2, 12.0, |
| | | 17.2 Hz, 1H), 2.52 (br s, 1H), 2.40-2.25 (m, 2H), 2.24-2.13 (m, 1H), 2.02 |
| | | (td, J = 4.4, 8.8 Hz, 1H), 1.98-1.88 (m, 4H) |
| I-706 | 798.5 | 12.14-11.85 (m, 1H), 11.79-10.68 (m, 1H), 7.73-7.69 (m, 1H), 7.43-7.36 |
| | | (m, 1H), 7.30 (s, 1H), 7.28 (s, 1H), 7.19-7.16 (m, 1H), 7.13-7.08 (m, 1H), |
| | | 6.94-6.91 (m, 1H), 6.85 (br s, 1H), 6.47-6.42 (m, 1H), 6.21-6.15 (m, 1H), |
| | | 6.11-6.05 (m, 1H), 5.45 (dd, J = 5.2, 12.4 Hz, 1H), 4.42-4.31 (m, 6H), 4.27 |
| | | (br s, 1H), 3.66-3.53 (m, 4H), 3.39 (br d, J = 3.2 Hz, 1H), 3.25 (br dd, J = 2.8, |
| | | 6.4 Hz, 1H), 3.14-3.07 (m, 4H), 3.01-2.94 (m, 3H), 2.92 (s, 4H), 2.81-2.74 |
| | | (m, 1H), 2.71-2.60 (m, 2H), 2.34-2.26 (m, 2H), 2.06 (br d, J = 5.2 Hz, 1H) |
| I-714 | 747.4 | 13.81-13.66 (m, 1H), 10.85-10.75 (m, 1H), 8.11 (d, J = 10.8 Hz, 1H), 7.82- |
| | | 7.73 (m, 1H), 7.69 (d, J = 12.0 Hz, 1H), 7.26-7.18 (m, 2H), 7.18-7.12 (m, |
| | | 1H), 7.05-6.97 (m, 2H), 6.97-6.86 (m, 2H), 6.27-6.16 (m, 1H), 4.62 (q, J = |
| | | 7.2 Hz, 2H), 4.37 (br d, J = 17.6 Hz, 2H), 3.80 (dd, J = 4.8, 11.6 Hz, 1H), 3.69- |
| | | 3.56 (m, 2H), 3.38 (s, 2H), 3.10 (q, J = 6.8 Hz, 2H), 2.70-2.61 (m, 5H), 2.48 |
| | | (s, 1H), 2.37-2.26 (m, 2H), 2.23-2.11 (m, 4H), 2.05-1.98 (m, 1H), 1.87 (d, |
| | | J = 10.8 Hz, 2H), 1.69-1.59 (m, 2H), 1.48-1.34 (m, 3H) |
| I-715 | 868.3 | 12.25-12.08 (m, 1H), 10.84 (br s, 1H), 7.77-7.63 (m, 1H), 7.45-7.35 (m, |
| | | 1H), 7.28 (br dd, J = 6.0, 12.4 Hz, 1H), 7.01-6.86 (m, 1H), 6.62-6.50 (m, |
| | | 3H), 6.22-6.11 (m, 3H), 4.90 (br d, J = 6.0 Hz, 1H), 4.39-4.34 (m, 3H), 4.28 |
| | | (br s, 1H), 4.21-4.14 (m, 5H), 3.97 (s, 4H), 3.78 (s, 3H), 3.66-3.55 (m, 2H), |
| | | 3.22-3.04 (m, 5H), 3.02-2.91 (m, 3H), 2.85-2.72 (m, 1H), 2.56 (br s, 1H), |
| | | 2.35-2.24 (m, 2H), 2.18-2.10 (m, 1H), 1.95-1.82 (m, 1H) |
| I-709 | 888.4 | 12.34-11.93 (m, 1H), 10.90-10.78 (m, 1H), 8.16-8.03 (m, 1H), 7.77-7.55 |
| | | (m, 1H), 7.36-7.09 (m, 3H), 7.02-6.71 (m, 3H), 6.44-6.30 (m, 1H), 6.19- |
| | | 6.07 (m, 1H), 5.70 (d, J = 7.6 Hz, 1H), 4.70-4.55 (m, 2H), 4.33 (s, 3H), 3.69- |
| | | 3.56 (m, 2H), 3.30-3.25 (m, 2H), 3.21-2.99 (m, 8H), 2.79-2.65 (m, 4H), |
| | | 2.60-2.56 (m, 1H), 2.35-2.26 (m, 2H), 2.15 (s, 3H), 2.10-2.01 (m, 2H), |
| | | 1.95-1.83 (m, 4H) |
| I-705 | 758.3 | 12.24-12.09 (m, 1H), 11.29-10.92 (m, 1H), 7.42 (s, 1H), 7.34 (s, 2H), 7.14- |
| | | 6.94 (m, 1H), 6.93-6.83 (m, 1H), 6.83-6.74 (m, 1H), 6.45 (br d, J = 2.0 Hz, |
| | | 1H), 6.33-6.27 (m, 1H), 6.20-6.15 (m, 1H), 5.35-5.28 (m, 1H), 4.36 (br d, J = |
| | | 6.8 Hz, 2H), 3.91-3.83 (m, 1H), 3.64 (br d, J = 6.4 Hz, 1H), 3.59 (s, 3H), |
| | | 3.16-3.02 (m, 6H), 2.90-2.83 (m, 1H), 2.72-2.62 (m, 2H), 2.39-2.37 (m, |
| | | 1H), 2.27 (br dd, J = 4.8, 6.8 Hz, 1H), 2.21 (br d, J = 6.0 Hz, 3H), 2.08 (br d, J = |
| | | 2.8 Hz, 4H), 2.01-1.95 (m, 6H), 1.70-1.62 (m, 2H), 1.23 (s, 2H) |
| I-713 | 763.4 | 13.74-13.46 (m, 1H), 10.88-10.77 (m, 1H), 8.15-8.07 (m, 1H), 7.85-7.80 |
| | | (m, 1H), 7.74-7.66 (m, 1H), 7.34 (s, 1H), 7.07-6.91 (m, 6H), 6.23-6.15 (m, |
| | | 1H), 4.70 (s, 2H), 4.44-4.32 (m, 2H), 3.83-3.78 (m, 1H), 3.78-3.72 (m, |
| | | 3H), 3.68-3.58 (m, 2H), 3.38 (br s, 1H), 3.14-3.06 (m, 2H), 2.80-2.55 (m, |
| | | 6H), 2.38-2.27 (m, 2H), 2.24-2.15 (m, 1H), 2.06-1.96 (m, 1H), 1.92-1.83 |
| | | (m, 2H), 1.71-1.62 (m, 2H), 1.24 (br s, 4H) |
| I-703 | 842.2 | 11.14-11.03 (m, 1H), 7.69 (dd, J = 2.0, 10.0 Hz, 1H), 7.55 (d, J = 1.6 Hz, |
| | | 1H), 7.47-7.38 (m, 5H), 7.05-7.00 (m, 1H), 7.00-6.97 (m, 2H), 6.90 (br d, J = |
| | | 7.6 Hz, 1H), 6.19 (td, J = 2.0, 7.6 Hz, 1H), 5.37 (dd, J = 5.2, 12.4 Hz, 1H), |
| | | 4.62-4.46 (m, 1H), 4.34 (q, J = 7.2 Hz, 2H), 4.05-3.88 (m, 1H), 3.71 (s, 3H), |
| | | 3.26 (br d, J = 11.6 Hz, 2H), 3.21 (d, J = 9.2 Hz, 3H), 3.11-3.03 (m, 1H), 3.00 |
| | | (s, 6H), 2.95-2.90 (m, 4H), 2.79 (br d, J = 8.8 Hz, 2H), 2.71 (br dd, J = 4.4, |
| | | 13.2 Hz, 1H), 2.67-2.58 (m, 2H), 2.10-1.92 (m, 8H), 1.80-1.72 (m, 1H), |
| | | 1.54-1.39 (m, 1H) |
| I-704 | 797.3 | 12.10 (br s, 1H), 10.65 (s, 1H), 8.18 (d, J = 6.8 Hz, 1H), 7.78-7.63 (m, 1H), |
| | | 7.55 (s, 1H), 7.51-7.33 (m, 3H), 7.17 (d, J = 6.8 Hz, 1H), 6.97-6.88 (m, 2H), |
| | | 6.84 (br s, 1H), 6.53 (d, J = 8.4 Hz, 2H), 6.25-6.09 (m, 2H), 4.37 (br d, J = |
| | | 6.4 Hz, 4H), 4.01 (t, J = 7.2 Hz, 2H), 3.85-3.76 (m, 2H), 3.67-3.52 (m, 4H), |
| | | 3.27-3.03 (m, 6H), 3.02-2.90 (m, 5H), 2.85-2.78 (m, 2H), 2.38-2.23 (m, |
| | | 2H), 2.11 (q, J = 7.2 Hz, 2H) |
| I-710 | 887.4 | 12.25-12.02 (m, 1H), 10.93-10.60 (m, 1H), 7.86-7.64 (m, 1H), 7.50-7.33 |
| | | (m, 1H), 7.29-7.16 (m, 3H), 7.00-6.79 (m, 3H), 6.44-6.37 (m, 1H), 6.26- |
| | | 6.08 (m, 2H), 5.74-5.61 (m, 1H), 4.48-4.41 (m, 1H), 4.40-4.28 (m, 4H), |
| | | 3.68-3.53 (m, 2H), 3.31 (s, 2H), 3.23-3.01 (m, 6H), 3.00-2.95 (m, 2H), |
| | | 2.81-2.71 (m, 3H), 2.58 (d, J = 3.6 Hz, 2H), 2.34-2.24 (m, 2H), 2.20-2.12 |
| | | (m, 3H), 2.11-2.00 (m, 2H), 1.96-1.81 (m, 4H) |
| I-712 | 839.3 | 12.21 (br d, J = 11.6 Hz, 1H), 10.89-10.82 (m, 1H), 7.73 (s, 1H), 7.52-7.47 |
| | | (m, 1H), 7.43 (br t, J = 5.2 Hz, 2H), 7.10-7.04 (m, 1H), 6.92-6.80 (m, 1H), |
| | | 6.71 (d, J = 8.0 Hz, 1H), 6.68 (br s, 1H), 6.59 (d, J = 14.0 Hz, 1H), 6.20 (br d, |
| | | J = 2.0 Hz, 1H), 5.14 (br d, J = 6.4 Hz, 1H), 4.60-4.52 (m, 2H), 4.38 (br t, J = |
| | | 6.8 Hz, 2H), 4.31-4.25 (m, 1H), 3.82 (s, 3H), 3.66-3.55 (m, 2H), 3.31-3.24 |
| | | (m, 6H), 3.12 (br s, 6H), 3.03-2.97 (m, 3H), 2.84-2.76 (m, 1H), 2.57 (br s, |
| | | 1H), 2.40-2.28 (m, 3H), 2.17-2.10 (m, 1H), 1.99-1.88 (m, 1H). |
| I-735 | 834.2 | 12.18-11.81 (m, 1H), 10.94-10.72 (m, 1H), 8.44-8.29 (m, 1H), 8.16-8.05 |
| | | (m, 1H), 7.91-7.81 (m, 1H), 7.74-7.59 (m, 1H), 7.48-7.41 (m, 1H), 7.28- |
| | | 7.20 (m, 1H), 7.02-6.97 (m, 1H), 6.94-6.84 (m, 2H), 6.50-6.39 (m, 1H), |
| | | 6.15-6.08 (m, 1H), 4.65-4.57 (m, 2H), 4.38-4.29 (m, 2H), 4.00-3.93 (m, |
| | | 2H), 3.87-3.81 (m, 1H), 3.74-3.69 (m, 3H), 3.67-3.57 (m, 2H), 3.21-2.99 |
| | | (m, 8H), 2.87-2.80 (m, 2H), 2.73-2.65 (m, 3H), 2.55 (br d, J = 3.2 Hz, 1H), |
| | | 2.36-2.32 (m, 1H), 2.31-2.17 (m, 2H), 2.03-1.95 (m, 1H), 1.88-1.80 (m, |
| | | 2H), 1.66-1.59 (m, 2H), 1.59-1.49 (m, 1H), 1.37-1.26 (m, 2H) |
| I-737 | 791.3 | 12.08 (s, 1H), 10.77 (s, 1H), 8.10 (d, J = 11.6 Hz, 1H), 7.69 (d, J = 10.8 Hz, |
| | | 1H), 7.61 (d, J = 8.0 Hz, 2H), 7.46 (d, J = 8.0 Hz, 2H), 7.08 (t, J = 5.2 Hz, |
| | | 1H), 7.00 (t, J = 8.8 Hz, 1H), 6.86 (s, 1H), 6.44 (d, J = 11.2 Hz, 2H), 5.98 (d, J = |
| | | 7.6 Hz, 1H), 4.61 (q, J = 7.2 Hz, 2H), 4.49 (d, J = 12.0 Hz, 1H), 4.36-4.23 |
| | | (m, 1H), 3.96-3.86 (m, 1H), 3.16 (dd, J = 2.4, 5.2 Hz, 2H), 3.13-3.02 (m, |
| | | 6H), 3.01-2.90 (m, 4H), 2.85-2.63 (m, 2H), 2.62-2.54 (m, 1H), 2.09 (dd, J = |
| | | 3.2, 7.6 Hz, 3H), 1.95-1.83 (m, 5H), 1.82-1.70 (m, 5H), 1.60-1.40 (m, |
| | | 1H) |
| I-762 | 856.3 | 12.26-12.19 (m, 1H), 10.91-10.85 (m, 1H), 8.14-8.10 (m, 1H), 7.71-7.68 |
| | | (m, 1H), 7.39 (br dd, J = 7.2, 12.8 Hz, 1H), 7.08-7.04 (m, 1H), 7.03-6.98 (m, |
| | | 1H), 6.80 (d, J = 7.2 Hz, 1H), 6.67-6.63 (m, 1H), 6.52 (d, J = 12.8 Hz, 1H), |
| | | 6.20-6.14 (m, 1H), 5.31 (br d, J = 6.8 Hz, 1H), 4.66-4.60 (m, 2H), 4.40- |
| | | 4.32 (m, 3H), 3.83 (s, 3H), 3.66 (br t, J = 6.0 Hz, 1H), 3.62-3.57 (m, 3H), |
| | | 3.22-3.17 (m, 2H), 3.13-3.07 (m, 4H), 2.89-2.81 (m, 4H), 2.58-2.56 (m, |
| | | 2H), 2.38-2.33 (m, 2H), 2.31-2.26 (m, 1H), 2.17-2.11 (m, 1H), 1.98-1.90 |
| | | (m, 3H), 1.81 (br d, J = 10.4 Hz, 2H) |
| I-763 | 820.4 | 12.17 (br d, J = 6.8 Hz, 1H), 10.87 (s, 1H), 8.46-8.40 (m, 1H), 7.85 (br d, J = |
| | | 8.4 Hz, 1H), 7.72 (dd, J = 1.8, 10.4 Hz, 1H), 7.45-7.36 (m, 1H), 7.05-6.95 |
| | | (m, 2H), 6.86 (br s, 1H), 6.72 (br d, J = 7.2 Hz, 1H), 6.51 (d, J = 12.8 Hz, 1H), |
| | | 6.20 (br d, J = 13.2 Hz, 1H), 6.16 (br s, 1H), 5.29 (br d, J = 7.2 Hz, 1H), 4.55 |
| | | (br d, J = 12.4 Hz, 2H), 4.41-4.36 (m, 3H), 4.35-4.27 (m, 2H), 3.78 (s, 3H), |
| | | 3.65 (br t, J = 5.6 Hz, 1H), 3.59 (br s, 1H), 3.25-3.19 (m, 2H), 3.10-3.04 (m, |
| | | 2H), 3.02-2.96 (m, 4H), 2.95-2.90 (m, 2H), 2.81 (br s, 1H), 2.57 (br d, J = |
| | | 2.4 Hz, 1H), 2.34 (br s, 1H), 2.30-2.25 (m, 1H), 2.17-2.09 (m, 1H), 1.95 (dt, |
| | | J = 8.8, 12.8 Hz, 1H), 1.86-1.65 (m, 5H) |
| I-764 | 877.4 | 12.38-11.84 (m, 1H), 11.00-10.65 (m, 1H), 7.77-7.66 (m, 1H), 7.47-7.34 |
| | | (m, 1H), 7.08-7.01 (m, 1H), 6.99-6.89 (m, 1H), 6.82-6.77 (m, 1H), 6.60- |
| | | 6.49 (m, 3H), 6.23-6.12 (m, 2H), 5.30 (d, J = 6.4 Hz, 1H), 4.40-4.28 (m, |
| | | 5H), 3.83 (s, 3H), 3.67-3.56 (m, 2H), 3.51 (d, J = 10.0 Hz, 2H), 3.27-3.01 |
| | | (m, 6H), 3.01-2.96 (m, 2H), 2.89-2.78 (m, 4H), 2.58 (d, J = 2.8 Hz, 1H), |
| | | 2.35-2.24 (m, 2H), 2.19-2.10 (m, 1H), 2.01-1.87 (m, 3H), 1.81 (d, J = 12.0 |
| | | Hz, 2H), 1.74-1.64 (m, 1H), 0.79-0.63 (m, 4H) |
| I-765 | 851.2 | 11.56-11.52 (m, 1H), 11.13-11.10 (m, 1H), 8.18-8.15 (m, 1H), 8.14-8.08 |
| | | (m, 1H), 7.72-7.66 (m, 1H), 7.25-7.20 (m, 1H), 7.17-7.13 (m, 1H), 7.01- |
| | | 6.97 (m, 2H), 6.96-6.93 (m, 1H), 6.87-6.78 (m, 1H), 6.12-6.06 (m, 1H), |
| | | 5.93-5.89 (m, 1H), 5.41-5.34 (m, 1H), 4.64-4.59 (m, 2H), 4.36-4.29 (m, |
| | | 2H), 3.66-3.63 (m, 1H), 3.61 (s, 3H), 3.59-3.57 (m, 1H), 3.13 (br s, 6H), |
| | | 3.10-3.07 (m, 2H), 2.92-2.85 (m, 1H), 2.73-2.66 (m, 7H), 2.37 (s, 3H), |
| | | 2.33 (br d, J = 2.0 Hz, 2H), 2.28-2.23 (m, 1H), 2.04-1.98 (m, 1H). |
| I-766 | 750.3 | 13.81-13.43 (m, 1H), 10.91-10.63 (m, 1H), 8.17-8.05 (m, 1H), 7.95-7.78 |
| | | (m, 1H), 7.74-7.62 (m, 1H), 7.36-7.26 (m, 1H), 7.13-7.08 (m, 1H), 7.04- |
| | | 6.89 (m, 3H), 6.62-6.51 (m, 1H), 6.49-6.42 (m, 1H), 6.25-6.15 (m, 1H), |
| | | 5.87-5.79 (m, 1H), 4.66-4.59 (m, 2H), 4.40-4.24 (m, 3H), 3.79-3.75 (m, |
| | | 3H), 3.67-3.59 (m, 2H), 3.29 (br s, 2H), 3.14-3.08 (m, 2H), 2.79-2.69 (m, |
| | | 4H), 2.61 (br t, J = 4.0 Hz, 1H), 2.38-2.34 (m, 1H), 2.32-2.24 (m, 1H), 2.15- |
| | | 2.05 (m, 1H), 1.98-1.86 (m, 5H) |
| I-767 | 887.4 | 12.19 (br d, J = 1.6 Hz, 1H), 10.86-10.76 (m, 1H), 7.83-7.75 (m, 1H), 7.30- |
| | | 7.25 (m, 2H), 7.10-7.06 (m, 1H), 7.00-6.93 (m, 2H), 6.92-6.88 (m, 2H), |
| | | 6.56-6.49 (m, 1H), 6.47-6.41 (m, 2H), 6.17-6.10 (m, 1H), 5.86-5.79 (m, |
| | | 1H), 4.36-4.30 (m, 2H), 4.30-4.23 (m, 1H), 3.80 (s, 7H), 3.76 (s, 3H), 3.64- |
| | | 3.57 (m, 2H), 3.27 (br d, J = 11.2 Hz, 2H), 2.70 (br s, 4H), 2.59 (br d, J = 4.0 |
| | | Hz, 2H), 2.54 (br s, 3H), 2.36 (br s, 1H), 2.26 (br s, 1H), 2.11 (br d, J = 4.4 Hz, |
| | | 1H), 2.08 (s, 2H), 2.07 (s, 1H), 1.94-1.89 (m, 4H), 1.88-1.83 (m, 1H) |
| I-768 | 863.4 | 12.27 (s, 1H), 10.95-10.73 (m, 1H), 7.74-7.68 (m, 1H), 7.45-7.36 (m, 2H), |
| | | 7.32 (d, J = 13.6 Hz, 1H), 7.13-7.00 (m, 1H), 6.67 (d, J = 8.0 Hz, 1H), 6.59 |
| | | (d, J = 14.4 Hz, 1H), 6.53 (s, 1H), 6.17 (dd, J = 14.0, 15.6 Hz, 2H), 5.15 (d, J = |
| | | 6.8 Hz, 1H), 4.42-4.24 (m, 5H), 3.82 (s, 3H), 3.67-3.56 (m, 2H), 3.49 (s, |
| | | 4H), 3.20-2.94 (m, 12H), 2.81 (ddd, J = 5.2, 13.2, 17.6 Hz, 1H), 2.59-2.52 |
| | | (m, 1H), 2.36-2.24 (m, 2H), 2.18-2.09 (m, 1H), 1.94 (dq, J = 4.0, 12.8 Hz, |
| | | 1H) |
| I-769 | 814.3 | 12.33 (br d, J = 6.8 Hz, 1H), 10.79 (s, 1H), 7.92 (s, 1H), 7.79-7.75 (m, 1H), |
| | | 7.72 (dd, J = 2.0, 12.8 Hz, 1H), 7.68 (d, J = 8.0 Hz, 1H), 7.46-7.36 (m, 1H), |
| | | 7.19-7.07 (m, 1H), 6.91 (t, J = 9.2 Hz, 1H), 6.66 (d, J = 2.8 Hz, 1H), 6.54 (dd, |
| | | J = 2.4, 14.8 Hz, 1H), 6.46 (dd, J = 1.8, 8.8 Hz, 1H), 6.19 (br dd, J = 1.8, 14.8 |
| | | Hz, 2H), 5.83 (d, J = 7.6 Hz, 1H), 4.41-4.35 (m, 3H), 4.33 (br d, J = 7.2 Hz, |
| | | 1H), 4.30-4.25 (m, 1H), 3.65 (br t, J = 5.6 Hz, 1H), 3.59 (br t, J = 5.6 Hz, |
| | | 1H), 3.31-3.24 (m, 3H), 3.19 (br s, 2H), 3.05 (br s, 2H), 3.02-2.92 (m, 3H), |
| | | 2.83-2.71 (m, 4H), 2.59 (td, J = 4.0, 17.2 Hz, 1H), 2.38-2.26 (m, 2H), 2.11 |
| | | (qd, J = 4.4, 12.8 Hz, 1H), 1.96-1.81 (m, 5H) |
| I-770 | 873.4 | 12.28-12.20 (m, 1H), 10.93-10.87 (m, 1H), 7.87-7.80 (m, 1H), 7.67-7.60 |
| | | (m, 2H), 7.56-7.48 (m, 1H), 7.20-7.14 (m, 2H), 7.11-7.03 (m, 1H), 6.93 (s, |
| | | 2H), 6.66-6.59 (m, 1H), 6.55-6.50 (m, 1H), 6.33-6.22 (m, 2H), 5.95-5.88 |
| | | (m, 1H), 4.51-4.46 (m, 3H), 4.45 (s, 2H), 3.76 (t, J = 5.6 Hz, 1H), 3.69 (d, J = |
| | | 11.6 Hz, 2H), 3.35-3.25 (m, 11H), 3.18 (s, 1H), 3.13-3.04 (m, 3H), 2.81 (d, J = |
| | | 4.4 Hz, 4H), 2.72 (s, 1H), 2.51-2.41 (m, 3H), 2.41-2.35 (m, 1H), 2.23- |
| | | 2.16 (m, 1H), 2.03-1.93 (m, 3H), 1.76-1.66 (m, 2H) |
| I-771 | 804.3 | 12.18 (d, J = 8.0 Hz, 1H), 10.36 (s, 1H), 7.74-7.69 (m, 1H), 7.59 (d, J = 8.0 |
| | | Hz, 2H), 7.45-7.35 (m, 3H), 7.18 (dd, J = 2.0, 14.0 Hz, 1H), 7.09-7.04 (m, |
| | | 2H), 7.03-6.97 (m, 1H), 6.85 (s, 1H), 6.22-6.13 (m, 2H), 4.39-4.30 (m, |
| | | 4H), 3.74 (t, J = 6.8 Hz, 2H), 3.65 (t, J = 5.6 Hz, 1H), 3.58 (t, J = 5.6 Hz, 1H), |
| | | 3.22-3.11 (m, 3H), 3.04 (s, 5H), 3.01-2.94 (m, 3H), 2.87-2.82 (m, 2H), |
| | | 2.72-2.68 (m, 2H), 2.66 (d, J = 9.2 Hz, 6H), 2.35-2.25 (m, 2H) |
| I-772 | 841.2 | 12.34-12.01 (m, 1H), 10.91-10.68 (m, 1H), 7.76-7.68 (m, 1H), 7.48-7.35 |
| | | (m, 1H), 7.34-7.26 (m, 1H), 7.23-7.17 (m, 1H), 7.12-7.04 (m, 1H), 7.00- |
| | | 6.90 (m, 1H), 6.52-6.45 (m, 3H), 6.22-6.17 (m, 1H), 6.14 (br d, J = 9.6 Hz, |
| | | 1H), 6.08-6.03 (m, 1H), 4.36 (q, J = 7.2 Hz, 4H), 4.29 (br s, 1H), 3.67-3.60 |
| | | (m, 1H), 3.59-3.53 (m, 3H), 3.23-3.11 (m, 3H), 3.10-3.00 (m, 3H), 2.97 (br |
| | | d, J = 7.2 Hz, 2H), 2.90-2.84 (m, 2H), 2.77-2.69 (m, 1H), 2.63-2.53 (m, |
| | | 2H), 2.34-2.31 (m, 1H), 2.31-2.21 (m, 1H), 2.13-2.05 (m, 1H), 1.94-1.84 |
| | | (m, 2H), 1.83-1.79 (m, 3H) |
| I-773 | 842.4 | 12.20 (br d, J = 7.2 Hz, 1H), 10.82-10.76 (m, 1H), 8.14-8.07 (m, 1H), 7.72- |
| | | 7.65 (m, 1H), 7.32-7.26 (m, 1H), 7.21-7.18 (m, 1H), 7.10-7.04 (m, 1H), |
| | | 7.00-6.90 (m, 1H), 6.51-6.44 (m, 3H), 6.18-6.12 (m, 1H), 6.05 (br d, J = |
| | | 7.6 Hz, 1H), 4.66-4.59 (m, 2H), 4.37-4.29 (m, 3H), 3.64 (br t, J = 5.6 Hz, |
| | | 1H), 3.61-3.53 (m, 3H), 3.22-3.13 (m, 3H), 3.09 (br d, J = 6.4 Hz, 2H), 3.07- |
| | | 2.98 (m, 3H), 2.88-2.83 (m, 2H), 2.76-2.69 (m, 1H), 2.61-2.52 (m, 2H), |
| | | 2.35 (br d, J = 2.0 Hz, 2H), 2.12-2.07 (m, 1H), 1.92-1.85 (m, 2H), 1.82 (br s, |
| | | 3H) |
| I-774 | 818.3 | 12.27-12.08 (m, 1H), 10.95-10.73 (m, 1H), 7.74-7.69 (m, 1H), 7.63 (br d, J = |
| | | 8.0 Hz, 2H), 7.48 (d, J = 8.4 Hz, 2H), 7.44-7.36 (m, 1H), 7.09-6.99 (m, |
| | | 1H), 6.88 (d, J = 2.4 Hz, 1H), 6.68 (br d, J = 7.2 Hz, 1H), 6.49 (d, J = 13.2 Hz, |
| | | 1H), 6.22-6.14 (m, 2H), 5.29-5.24 (m, 1H), 4.37 (br t, J = 6.8 Hz, 3H), 4.32- |
| | | 4.26 (m, 2H), 3.77-3.74 (m, 3H), 3.66-3.57 (m, 2H), 3.23-3.09 (m, 4H), |
| | | 3.06-2.94 (m, 6H), 2.84-2.76 (m, 1H), 2.58-2.53 (m, 1H), 2.35-2.26 (m, |
| | | 2H), 2.15-2.08 (m, 3H), 1.95-1.80 (m, 5H), 1.78-1.72 (m, 2H). |
| I-775 | 838.3 | 12.25-12.17 (m, 1H), 10.87 (s, 1H), 8.12 (dd, J = 0.4, 12.0 Hz, 1H), 7.72- |
| | | 7.64 (m, 3H), 7.51 (d, J = 8.4 Hz, 2H), 7.11-7.02 (m, 1H), 6.90-6.87 (m, |
| | | 1H), 6.77 (br d, J = 8.0 Hz, 1H), 6.61 (d, J = 14.0 Hz, 1H), 6.21-6.14 (m, 1H), |
| | | 5.09-4.96 (m, 1H), 4.94-4.86 (m, 1H), 4.64 (br d, J = 6.8 Hz, 3H), 4.39 (br s, |
| | | 1H), 4.33 (br d, J = 2.0 Hz, 2H), 3.83 (s, 3H), 3.67-3.59 (m, 3H), 3.25 (br s, |
| | | 2H), 3.21-3.15 (m, 2H), 3.13-3.06 (m, 4H), 3.00-2.83 (m, 4H), 2.83-2.75 |
| | | (m, 1H), 2.60-2.55 (m, 1H), 2.37-2.25 (m, 2H), 2.16-2.09 (m, 1H), 2.00- |
| | | 1.96 (m, 2H) |
| I-776 | 839.3 | 12.2-12.1 (m, 1H), 10.90 (br s, 1H), 7.75-7.68 (m, 1H), 7.47-7.36 (m, 2H), |
| | | 7.05 (d, J = 13.6 Hz, 1H), 6.99-6.92 (m, 3H), 6.74 (d, J = 8.4 Hz, 1H), 6.62 |
| | | (br s, 1H), 6.22-6.10 (m, 2H), 5.01 (dd, J = 5.2, 10.0 Hz, 1H), 4.40-4.28 (m, |
| | | 4H), 3.79 (s, 3H), 3.66-3.56 (m, 2H), 3.41 (br s, 4H), 3.19-3.05 (m, 9H), |
| | | 3.03-2.93 (m, 3H), 2.67-2.60 (m, 2H), 2.35-2.25 (m, 2H), 2.21-2.07 (m, |
| | | 2H). |
| I-777 | 853.5 | 12.42-12.10 (m, 1H), 11.10 (s, 1H), 7.44-7.31 (m, 1H), 7.09-6.91 (m, 5H), |
| | | 6.72-6.60 (m, 1H), 6.20-6.10 (m, 1H), 5.44-5.31 (m, 1H), 4.46-4.30 (m, |
| | | 2H), 3.78-3.61 (m, 6H), 3.22-2.88 (m, 12H), 2.86-2.55 (m, 8H), 2.41-2.36 |
| | | (m, 1H), 2.33-2.18 (m, 1H), 2.10-1.95 (m, 1H), 1.10-1.00 (m, 9H) |
| I-778 | 857.0 | 12.34-12.13 (m, 1H), 10.93-10.77 (m, 1H), 8.27 (s, 1H), 7.76-7.66 (m, |
| | | 1H), 7.47-7.33 (m, 1H), 7.12-7.01 (m, 1H), 6.75 (s, 1H), 6.71-6.67 (m, |
| | | 1H), 6.59 (d, J = 14.4 Hz, 1H), 6.25-6.12 (m, 2H), 5.19-5.10 (m, 1H), 4.39- |
| | | 4.34 (m, 3H), 4.34-4.24 (m, 2H), 3.82 (s, 3H), 3.69 (s, 4H), 3.66-3.62 (m, |
| | | 1H), 3.60-3.57 (m, 1H), 3.19 (s, 3H), 3.09 (s, 6H), 2.98 (q, J = 7.2 Hz, 3H), |
| | | 2.88-2.76 (m, 1H), 2.61-2.53 (m, 1H), 2.37-2.26 (m, 2H), 2.20-2.10 (m, |
| | | 1H), 1.94 (dq, J = 4.4, 12.8 Hz, 1H) |
| I-779 | 811.3 | 12.24-12.16 (m, 1H), 10.86-10.79 (m, 1H), 7.73 (d, J = 1.6 Hz, 1H), 7.44- |
| | | 7.35 (m, 2H), 7.12-7.05 (m, 3H), 7.03-6.97 (m, 2H), 6.67-6.61 (m, 1H), |
| | | 6.22-6.12 (m, 2H), 4.39-4.29 (m, 4H), 3.86-3.79 (m, 1H), 3.66-3.56 (m, |
| | | 2H), 3.29 (s, 6H), 3.20 (s, 6H), 3.09-3.03 (m, 2H), 3.02-2.93 (m, 3H), 2.71- |
| | | 2.62 (m, 1H), 2.34-2.25 (m, 2H), 2.24-2.17 (m, 1H), 2.06-1.98 (m, 1H) |
| I-780 | 922.4 | 12.18-12.11 (m, 1H), 10.86 (s, 1H), 8.15-8.08 (m, 1H), 7.73-7.66 (m, 1H), |
| | | 7.43-7.36 (m, 1H), 7.00-6.91 (m, 1H), 6.88 (br d, J = 11.2 Hz, 2H), 6.62 (br |
| | | d, J = 8.0 Hz, 2H), 6.55 (d, J = 14.4 Hz, 1H), 6.17-6.11 (m, 1H), 5.10 (br d, J = |
| | | 6.8 Hz, 1H), 4.66-4.59 (m, 2H), 4.40-4.31 (m, 2H), 4.30-4.22 (m, 1H), |
| | | 3.79 (s, 3H), 3.68-3.58 (m, 2H), 3.30-3.19 (m, 8H), 3.14-3.00 (m, 6H), |
| | | 2.81 (ddd, J = 5.2, 13.2, 17.6 Hz, 1H), 2.68 (br d, J = 4.8 Hz, 6H), 2.56 (br d, J = |
| | | 3.2 Hz, 1H), 2.37-2.33 (m, 2H), 2.28 (br d, J = 3.2 Hz, 1H), 2.18-2.09 (m, |
| | | 1H), 1.98-1.86 (m, 3H), 1.67-1.56 (m, 2H) |
| I-781 | 461.3^{b} | 12.19-12.10 (m, 1H), 10.87 (s, 1H), 7.75-7.69 (m, 1H), 7.45-7.36 (m, 2H), |
| | | 6.99-6.91 (m, 1H), 6.88 (br d, J = 11.6 Hz, 2H), 6.64-6.59 (m, 2H), 6.55 (d, |
| | | J = 14.4 Hz, 1H), 6.22-6.16 (m, 1H), 6.15-6.09 (m, 1H), 5.10 (d, J = 6.4 Hz, |
| | | 1H), 4.39-4.35 (m, 3H), 4.34-4.23 (m, 2H), 3.79 (s, 3H), 3.66-3.56 (m, |
| | | 2H), 3.28-3.19 (m, 8H), 3.08 (br d, J = 19.2 Hz, 3H), 3.01-2.93 (m, 3H), |
| | | 2.81 (s, 1H), 2.68 (br d, J = 4.4 Hz, 6H), 2.56 (br d, J = 3.2 Hz, 1H), 2.37- |
| | | 2.31 (m, 2H), 2.27 (br d, J = 2.0 Hz, 1H), 2.17-2.09 (m, 1H), 1.98-1.86 (m, |
| | | 3H), 1.67-1.54 (m, 2H). |
| I-782 | 859.4 | 12.18-12.06 (m, 1H), 10.83 (br s, 1H), 8.68 (d, J = 8.0 Hz, 1H), 7.85 (d, J = |
| | | 8.4 Hz, 1H), 7.82 (d, J = 2.4 Hz, 1H), 7.75-7.66 (m, 1H), 7.47-7.31 (m, 2H), |
| | | 6.97-6.89 (m, 4H), 6.61 (br d, J = 2.0 Hz, 1H), 6.22-6.08 (m, 2H), 4.74 (ddd, |
| | | J = 5.2, 7.6, 12.8 Hz, 1H), 4.39-4.28 (m, 4H), 3.81 (s, 4H), 3.64 (br t, J = 5.2 |
| | | Hz, 1H), 3.58 (br t, J = 5.6 Hz, 1H), 3.39-3.34 (m, 2H), 3.21-3.02 (m, 6H), |
| | | 3.02-2.92 (m, 3H), 2.83-2.74 (m, 1H), 2.53 (br d, J = 3.2 Hz, 1H), 2.33 (br s, |
| | | 2H), 2.26 (br d, J = 2.8 Hz, 1H), 2.17 (br dd, J = 4.4, 12.8 Hz, 1H), 2.04-1.98 |
| | | (m, 1H), 1.91 (br s, 4H) |
| I-783 | 877.3 | 12.23 (br d, J = 1.6 Hz, 1H), 10.85 (br s, 1H), 8.69 (d, J = 8.0 Hz, 1H), 7.91- |
| | | 7.80 (m, 2H), 7.76-7.67 (m, 1H), 7.48-7.35 (m, 1H), 7.30 (br s, 1H), 7.04- |
| | | 6.90 (m, 3H), 6.66 (br d, J = 2.4 Hz, 1H), 6.23-6.10 (m, 2H), 4.81-4.68 (m, |
| | | 1H), 4.39-4.27 (m, 4H), 3.83 (s, 4H), 3.70-3.54 (m, 2H), 3.12 (br s, 6H), |
| | | 3.07-2.94 (m, 5H), 2.86-2.74 (m, 1H), 2.54 (br d, J = 4.0 Hz, 1H), 2.38- |
| | | 2.22 (m, 3H), 2.18 (br dd, J = 4.4, 12.8 Hz, 1H), 1.99 (br s, 5H) |
| I-784 | 877.4 | 12.20 (br s, 1H), 10.83 (br s, 1H), 8.68 (d, J = 8.0 Hz, 1H), 7.88-7.82 (m, 2H), |
| | | 7.74-7.68 (m, 1H), 7.45-7.31 (m, 2H), 7.04-6.96 (m, 2H), 6.93 (dd, J = 2.4, |
| | | 8.4 Hz, 1H), 6.63 (br s, 1H), 6.21-6.11 (m, 2H), 4.78-4.70 (m, 1H), 4.39- |
| | | 4.34 (m, 3H), 4.31 (br d, J = 20.0 Hz, 1H), 3.81 (s, 4H), 3.66-3.56 (m, 2H), |
| | | 3.21-3.14 (m, 2H), 3.10 (br s, 6H), 3.06-3.03 (m, 1H), 3.02-2.91 (m, 3H), |
| | | 2.84-2.74 (m, 1H), 2.54 (br d, J = 3.2 Hz, 1H), 2.33 (br s, 1H), 2.29-2.22 (m, |
| | | 1H), 2.21-2.12 (m, 1H), 2.02 (br d, J = 5.2 Hz, 1H), 1.96 (br s, 4H). |
| I-785 | 878.4 | 12.20 (br d, J = 0.8 Hz, 1H), 10.83 (s, 1H), 8.68 (d, J = 8.4 Hz, 1H), 8.13-8.08 |
| | | (m, 1H), 7.87-7.82 (m, 2H), 7.73-7.64 (m, 1H), 7.40-7.32 (m, 1H), 7.04- |
| | | 6.96 (m, 2H), 6.93 (dd, J = 2.4, 8.4 Hz, 1H), 6.63 (br d, J = 2.0 Hz, 1H), 6.18- |
| | | 6.12 (m, 1H), 4.77-4.70 (m, 1H), 4.62 (q, J = 7.2 Hz, 2H), 4.38-4.30 (m, |
| | | 2H), 3.81 (s, 4H), 3.67-3.58 (m, 2H), 3.21-3.15 (m, 2H), 3.09 (br d, J = 6.4 |
| | | Hz, 6H), 3.06 (br s, 2H), 3.05-2.97 (m, 2H), 2.84-2.74 (m, 1H), 2.54 (br d, J = |
| | | 3.2 Hz, 1H), 2.35 (br d, J = 0.8 Hz, 1H), 2.27 (br s, 1H), 2.18 (dt, J = 8.8, |
| | | 12.8 Hz, 1H), 2.02 (br d, J = 5.2 Hz, 1H), 1.96 (br s, 4H) |
| I-1094 | 775.3 | 12.28 (br s, 1H), 11.13-11.06 (m, 1H), 8.84 (br s, 1H), 8.09-8.01 (m, 1H), |
| | | 7.49 (br d, J = 8.4 Hz, 1H), 7.16-7.08 (m, 1H), 7.04-6.96 (m, 2H), 6.90 (br |
| | | d, J = 3.2 Hz, 2H), 6.20 (br s, 1H), 5.37 (br dd, J = 5.6, 12.4 Hz, 1H), 4.44- |
| | | 4.37 (m, 2H), 3.70 (s, 3H), 3.67 (br d, J = 5.6 Hz, 2H), 3.28-3.24 (m, 6H), |
| | | 3.08-3.03 (m, 2H), 2.96-2.87 (m, 5H), 2.71 (br d, J = 8.8 Hz, 1H), 2.65 (br s, |
| | | 1H), 2.60 (br s, 1H), 2.39 (br s, 1H), 2.27 (br s, 1H), 2.09-2.00 (m, 4H), 1.07- |
| | | 0.99 (m, 6H) |
| I-1095 | 872.3 | 12.34-12.02 (m, 1H), 11.00-10.53 (m, 1H), 8.15-8.03 (m, 1H), 7.74-7.61 |
| | | (m, 1H), 7.31 (dd, J = 5.6, 13.2 Hz, 1H), 7.21 (d, J = 8.4 Hz, 1H), 7.02-6.87 |
| | | (m, 1H), 6.79 (d, J = 6.8 Hz, 1H), 6.57-6.44 (m, 2H), 6.20-6.07 (m, 1H), |
| | | 5.30 (d, J = 7.2 Hz, 1H), 4.62 (q, J = 7.2 Hz, 2H), 4.40-4.27 (m, 3H), 3.82 (s, |
| | | 3H), 3.69-3.53 (m, 4H), 3.21-3.01 (m, 8H), 2.92-2.77 (m, 4H), 2.57 (d, J = |
| | | 1.2 Hz, 1H), 2.37-2.26 (m, 2H), 2.19-2.08 (m, 1H), 2.01-1.87 (m, 3H), 1.81 |
| | | (d, J = 11.2 Hz, 2H) |
| I-1096 | 855.3 | 12.22 (s, 1H), 11.09 (s, 1H), 7.60 (d, J = 8.0 Hz, 1H), 7.16 (d, J = 12.0 Hz, |
| | | 1H), 7.05-6.90 (m, 4H), 6.63-6.59 (m, 1H), 6.16 (s, 1H), 5.38 (dd, J = 5.6, |
| | | 12.4 Hz, 1H), 4.42-4.32 (m, 2H), 3.66 (d, J = 6.0 Hz, 2H), 3.63-3.59 (m, |
| | | 4H), 3.11 (s, 11H), 3.03-2.83 (m, 4H), 2.77-2.60 (m, 8H), 2.42-2.30 (m, |
| | | 2H), 2.25 (s, 1H), 2.06-1.95 (m, 1H), 1.10-0.96 (m, 6H) |
| I-1097 | 821.3 | 12.31-11.92 (m, 1H), 10.86 (br s, 1H), 8.51 (br s, 1H), 7.89-7.78 (m, 1H), |
| | | 7.77-7.66 (m, 1H), 7.49 (br d, J = 9.2 Hz, 1H), 7.46-7.37 (m, 2H), 7.37- |
| | | 7.30 (m, 1H), 6.69 (d, J = 8.0 Hz, 1H), 6.60 (d, J = 14.4 Hz, 1H), 6.25-6.13 |
| | | (m, 2H), 5.15 (d, J = 6.8 Hz, 1H), 4.42-4.26 (m, 5H), 3.82 (s, 3H), 3.69-3.57 |
| | | (m, 2H), 3.44 (br s, 4H), 3.39-3.35 (m, 1H), 3.24-3.05 (m, 9H), 3.02-2.96 |
| | | (m, 2H), 2.82 (ddd, J = 5.2, 13.2, 18.0 Hz, 1H), 2.60-2.53 (m, 1H), 2.40- |
| | | 2.24 (m, 2H), 2.21-2.10 (m, 1H), 1.94 (dq, J = 4.4, 12.8 Hz, 1H) |
| I-1098 | 839.0 | 12.17-12.09 (m, 1H), 10.87-10.81 (m, 1H), 8.38 (br s, 1H), 7.74-7.69 (m, |
| | | 1H), 7.45-7.37 (m, 2H), 7.27-7.22 (m, 1H), 6.97-6.92 (m, 1H), 6.68 (d, J = |
| | | 8.0 Hz, 1H), 6.59 (d, J = 14.4 Hz, 1H), 6.21-6.17 (m, 1H), 6.11 (br s, 1H), |
| | | 5.14 (d, J = 7.2 Hz, 1H), 4.37 (br d, J = 6.4 Hz, 3H), 4.32-4.25 (m, 2H), 3.82 |
| | | (s, 3H), 3.65 (br t, J = 5.2 Hz, 1H), 3.59 (br t, J = 5.6 Hz, 1H), 3.49 (br s, 4H), |
| | | 3.21-3.16 (m, 2H), 3.08 (br s, 6H), 2.99 (br t, J = 6.8 Hz, 2H), 2.96-2.91 (m, |
| | | 1H), 2.85-2.76 (m, 1H), 2.57 (br d, J = 2.8 Hz, 1H), 2.36-2.30 (m, 2H), 2.29- |
| | | 2.23 (m, 1H), 2.18-2.10 (m, 1H), 1.99-1.87 (m, 1H) |
| I-1099 | 839.3 | 12.31-11.92 (m, 1H), 10.86 (br s, 1H), 8.51 (br s, 1H), 7.89-7.78 (m, 1H), |
| | | 7.77-7.66 (m, 1H), 7.49 (br d, J = 9.2 Hz, 1H), 7.46-7.37 (m, 2H), 7.37- |
| | | 7.30 (m, 1H), 6.69 (d, J = 8.0 Hz, 1H), 6.60 (d, J = 14.4 Hz, 1H), 6.25-6.13 |
| | | (m, 2H), 5.15 (d, J = 6.8 Hz, 1H), 4.42-4.26 (m, 5H), 3.82 (s, 3H), 3.69-3.57 |
| | | (m, 2H), 3.44 (br s, 4H), 3.39-3.35 (m, 1H), 3.24-3.05 (m, 9H), 3.02-2.96 |
| | | (m, 2H), 2.82 (ddd, J = 5.2, 13.2, 18.0 Hz, 1H), 2.60-2.53 (m, 1H), 2.40- |
| | | 2.24 (m, 2H), 2.21-2.10 (m, 1H), 1.94 (dq, J = 4.4, 12.8 Hz, 1H) |
| I-110 | 776.4 | 12.17 (d, J = 2.0 Hz, 1H), 10.82 (s, 1H), 8.45 (d, J = 2.4 Hz, 1H), 7.89 (d, J = |
| | | 8.8 Hz, 1H), 7.72 (J = 2.0, 10.8 Hz, 1H), 7.45-7.35 (m, 1H), 7.10-6.97 (m, |
| | | 5H), 6.86 (d, J = 2.8 Hz, 1H), 6.22-6.13 (m, 2H), 4.39-4.29 (m, 4H), 3.83 (J = |
| | | 4.8, 11.6 Hz, 1H), 3.73 (s, 4H), 3.66-3.56 (m, 2H), 3.14 (s, 10H), 2.98 (q, J = |
| | | 6.8 Hz, 3H), 2.71-2.62 (m, 1H), 2.36-2.26 (m, 2H), 2.25-2.16 (m, 1H), |
| | | 2.01 (td, J = 4.4, 8.8 Hz, 1H). |
| I-1101 | 811.4 | 12.60-11.92 (m, 1H), 10.83 (s, 1H), 7.81-7.64 (m, 1H), 7.48-7.25 (m, 2H), |
| | | 7.19-6.98 (m, 5H), 6.75-6.63 (m, 1H), 6.23-6.02 (m, 2H), 4.44-4.29 (m, |
| | | 4H), 3.90-3.76 (m, 1H), 3.70-3.51 (m, 2H), 3.31-3.27 (m, 4H), 3.25-3.03 |
| | | (m, 10H), 3.00-2.95 (m, 2H), 2.71-2.63 (m, 1H), 2.42-2.11 (m, 4H), 2.07- |
| | | 1.96 (m, 1H) |
| I-1102 | 775.4 | 12.42-12.09 (m, 1H), 10.82 (s, 1H), 8.82 (br s, 1H), 8.04 (dd, J = 2.4, 8.0 Hz, |
| | | 1H), 7.72 (dd, J = 2.0, 10.8 Hz, 1H), 7.52-7.45 (m, 1H), 7.45-7.35 (m, 1H), |
| | | 7.16-6.96 (m, 4H), 6.88 (br d, J = 2.4 Hz, 1H), 6.25-6.12 (m, 2H), 4.41- |
| | | 4.30 (m, 4H), 3.82 (dd, J = 4.8, 12.0 Hz, 1H), 3.68-3.56 (m, 2H), 3.50 (br d, J = |
| | | 11.6 Hz, 2H), 3.27-3.03 (m, 6H), 3.02-2.78 (m, 6H), 2.72-2.61 (m, 1H), |
| | | 2.36-2.25 (m, 2H), 2.24-2.14 (m, 1H), 2.05-1.93 (m, 5H). |
| I-1103 | 894.7 | 12.26-12.18 (m, 1H), 10.81 (s, 1H), 7.74-7.69 (m, 1H), 7.46-7.35 (m, 1H), |
| | | 7.31-7.24 (m, 1H), 7.05-6.95 (m, 5H), 6.65 (s, 1H), 6.21-6.11 (m, 2H), |
| | | 4.39-4.29 (m, 4H), 3.80 J = 4.8, 12.0 Hz, 1H), 3.64-3.52 (m, 4H), 3.22- |
| | | 3.12 (m, 4H), 3.06-2.96 (m, 8H), 2.84-2.77 (m, 2H), 2.74-2.62 (m, 6H), |
| | | 2.36-2.30 (m, 2H), 2.27-2.16 (m, 2H), 2.03-1.91 (m, 3H), 1.69-1.59 (m, |
| | | 2H). |
| I-1104 | 775.4 | 12.16 (s, 1H), 10.81 (s, 1H), 8.70-8.62 (m, 1H), 7.94-7.82 (m, 2H), 7.75- |
| | | 7.68 (m, 1H), 7.53-7.45 (m, 1H), 7.45-7.35 (m, 1H), 7.35-7.27 (m, 1H), |
| | | 7.10-7.01 (m, 2H), 7.01-6.94 (m, 1H), 6.23-6.11 (m, 2H), 4.42-4.32 (m, |
| | | 4H), 3.86-3.77 (m, 1H), 3.69-3.56 (m, 2H), 3.50 (d, J = 11.6 Hz, 2H), 3.10- |
| | | 3.04 (m, 2H), 3.03-2.93 (m, 3H), 2.93-2.74 (m, 4H), 2.71-2.59 (m, 2H), |
| | | 2.40-2.30 (m, 2H), 2.30-2.24 (m, 1H), 2.21 (dd, J = 3.2, 12.0 Hz, 1H), 2.12- |
| | | 1.83 (m, 6H) |
| I-1105 | 837.3 | 12.33-12.08 (m, 1H), 10.85 (br s, 1H), 7.76-7.68 (m, 1H), 7.46-7.23 (m, |
| | | 3H), 7.09-6.92 (m, 4H), 6.63 (br d, J = 2.8 Hz, 1H), 6.25-6.09 (m, 2H), 4.41- |
| | | 4.27 (m, 4H), 3.87 (dd, J = 4.8, 11.9 Hz, 1H), 3.70-3.54 (m, 2H), 3.48 (br d, |
| | | J = 11.2 Hz, 2H), 3.21-2.94 (m, 8H), 2.79-2.59 (m, 6H), 2.35-2.31 (m, 1H), |
| | | 2.29-2.18 (m, 2H), 2.08-1.96 (m, 1H), 1.87 (br d, J = 10.4 Hz, 2H), 1.62- |
| | | 1.51 (m, 2H), 1.47-1.32 (m, 3H). |
| I-1106 | 839.4 | 12.33-12.02 (m, 1H), 11.01-10.72 (m, 1H), 8.17-8.06 (m, 1H), 7.81-7.62 |
| | | (m, 1H), 7.38-7.25 (m, 2H), 7.07-6.95 (m, 4H), 6.67-6.60 (m, 1H), 6.21- |
| | | 6.10 (m, 1H), 4.66-4.59 (m, 2H), 4.40-4.30 (m, 2H), 3.87 (dd, J = 4.8, 12.0 |
| | | Hz, 1H), 3.67-3.57 (m, 2H), 3.48 (br d, J = 10.8 Hz, 2H), 3.22-3.02 (m, 8H), |
| | | 2.77-2.62 (m, 6H), 2.36 (br s, 1H), 2.30-2.18 (m, 2H), 2.06-1.99 (m, 1H), |
| | | 1.86 (br d, J = 11.2 Hz, 2H), 1.61-1.53 (m, 2H), 1.45-1.32 (m, 3H). |
| I-1107 | 939.4 | 12.22 (d, J = 8.0 Hz, 1H), 10.79 (s, 1H), 7.72 (d, J = 1.6 Hz, 1H), 7.48-7.35 |
| | | (m, 1H), 7.33-7.21 (m, 1H), 7.09-6.92 (m, 3H), 6.90-6.83 (m, 1H), 6.72- |
| | | 6.62 (m, 1H), 6.57-6.42 (m, 1H), 6.25-6.05 (m, 2H), 4.41-4.28 (m, 4H), |
| | | 3.78-3.72 (m, 4H), 3.68-3.61 (m, 1H), 3.60-3.56 (m, 1H), 3.21-3.03 (m, |
| | | 9H), 3.01-2.93 (m, 2H), 2.69-2.60 (m, 1H), 2.57-2.53 (m, 1H), 2.49-2.44 |
| | | (m, 1H), 2.36-2.25 (m, 2H), 2.22 (s, 1H), 2.01 (d, J = 4.0 Hz, 1H), 1.96 (s, |
| | | 4H) |
| I-1108 | 799.5 | 12.02 (s, 1H), 10.46-10.41 (m, 1H), 8.03 (s, 1H), 7.74-7.68 (m, 2H), 7.54 (d, |
| | | J = 8.8 Hz, 2H), 7.45-7.39 (m, 2H), 7.14 (d, J = 8.4 Hz, 2H), 7.09-7.00 (m, |
| | | 2H), 6.87 (s, 1H), 6.24-6.16 (m, 1H), 4.98-4.89 (m, 1H), 4.52-4.46 (m, |
| | | 1H), 4.36 (q, J = 6.4 Hz, 2H), 4.00 (d, J = 12.0 Hz, 2H), 3.88 (t, J = 6.4 Hz, |
| | | 3H), 3.12 (s, 10H), 2.78 (t, J = 6.4 Hz, 3H), 2.70-2.53 (m, 1H), 2.31-2.20 |
| | | (m, 2H), 2.08-1.84 (m, 5H), 1.80-1.73 (m, 1H), 1.55-1.41 (m, 1H). |
| I-1109 | 880.4 | 12.22-11.97 (m, 1H), 10.78-10.56 (m, 1H), 8.21-8.11 (m, 1H), 7.75-7.68 |
| | | (m, 1H), 7.56 (s, 1H), 7.46-7.37 (m, 1H), 7.32-7.25 (m, 1H), 7.23-7.13 (m, |
| | | 2H), 7.02-6.96 (m, 1H), 6.94-6.87 (m, 1H), 6.53-6.44 (m, 1H), 6.24 (s, |
| | | 1H), 4.55-4.43 (m, 1H), 4.41-4.31 (m, 2H), 3.93-3.84 (m, 1H), 3.81 (br t, J = |
| | | 6.6 Hz, 2H), 3.15 (br s, 8H), 3.10-3.02 (m, 4H), 2.96-2.90 (m, 2H), 2.89- |
| | | 2.76 (m, 5H), 2.74-2.62 (m, 5H), 1.98-1.82 (m, 2H), 1.80-1.72 (m, 1H), |
| | | 1.55-1.38 (m, 1H). |
| I-1110 | 880.3 | 12.09 (br s, 1H), 10.46 (br s, 1H), 8.04 (s, 1H), 7.71 (dd, J = 2.0, 9.6 Hz, 1H), |
| | | 7.66 (d, J = 8.4 Hz, 1H), 7.48-7.37 (m, 2H), 7.27 (dd, J = 6.8, 13.2 Hz, 1H), |
| | | 7.14 (d, J = 8.4 Hz, 1H), 7.06 (d, J = 7.2 Hz, 1H), 6.96 (dd, J = 2.8, 5.6 Hz, |
| | | 1H), 6.48 (d, J = 3.2 Hz, 1H), 6.20 (td, J = 2.0, 10.8 Hz, 1H), 4.67-4.53 (m, |
| | | 2H), 4.53-4.44 (m, 1H), 4.39-4.30 (m, 2H), 3.90 (br t, J = 6.8 Hz, 3H), 3.25- |
| | | 2.99 (m, 12H), 2.95-2.85 (m, 4H), 2.80 (t, J = 6.8 Hz, 2H), 2.68 (br s, 5H), |
| | | 1.97-1.72 (m, 3H), 1.57-1.36 (m, 1H). |
| I-1111 | 911.4 | 12.46-11.98 (m, 1H), 10.71-10.33 (m, 1H), 7.83-7.76 (m, 1H), 7.74-7.61 |
| | | (m, 3H), 7.49-7.23 (m, 2H), 7.09-6.89 (m, 2H), 6.75-6.56 (m, 1H), 6.26- |
| | | 6.07 (m, 2H), 4.39-4.33 (m, 4H), 4.31-4.20 (m, 2H), 4.17-4.09 (m, 1H), |
| | | 3.97-3.90 (m, 1H), 3.86-3.72 (m, 2H), 3.65-3.56 (m, 4H), 3.22-3.09 (m, |
| | | 8H), 3.08-3.03 (m, 2H), 3.00-2.90 (m, 3H), 2.89-2.69 (m, 4H), 2.36-2.24 |
| | | (m, 2H). |
| I-1112 | 810.2 | 12.32-12.16 (m, 1H), 10.90-10.81 (m, 1H), 8.49-8.46 (m, 1H), 7.78-7.69 |
| | | (m, 1H), 7.43 (s, 2H), 7.33-7.28 (m, 1H), 7.10-6.98 (m, 4H), 6.69-6.64 (m, |
| | | 1H), 6.23 (s, 2H), 4.40-4.36 (m, 3H), 4.34-4.28 (m, 1H), 3.91-3.86 (m, |
| | | 1H), 3.68-3.54 (m, 5H), 3.22-3.07 (m, 6H), 3.02-2.91 (m, 6H), 2.34-2.32 |
| | | (m, 1H), 2.29-2.23 (m, 2H), 2.06-2.01 (m, 1H), 1.95-1.86 (m, 4H) |
| I-1113 | 902.5 | 12.34-11.88 (m, 1H), 10.66-10.24 (m, 1H), 7.78-7.65 (m, 1H), 7.52 (d, J = |
| | | 8.4 Hz, 2H), 7.46-7.33 (m, 3H), 7.08 (d, J = 8.6 Hz, 2H), 6.99-6.90 (m, 1H), |
| | | 6.84 (s, 1H), 6.26-6.05 (m, 2H), 4.41-4.35 (m, 3H), 4.32 (s, 1H), 3.77-3.70 |
| | | (m, 1H), 3.69-3.61 (m, 3H), 3.61-3.54 (m, 2H), 3.24 (d, J = 1.6 Hz, 6H), |
| | | 3.16 (s, 4H), 3.04 (s, 2H), 3.00-2.94 (m, 2H), 2.80-2.70 (m, 2H), 2.44 (s, |
| | | 3H), 2.36-2.24 (m, 2H), 1.64 (s, 4H), 1.58-1.43 (m, 4H) |
| I-1114 | 838.4 | 12.33-12.07 (m, 1H), 10.84 (s, 1H), 7.76-7.66 (m, 1H), 7.50-7.33 (m, 1H), |
| | | 7.31-7.20 (m, 2H), 7.07-7.00 (m, 2H), 7.00-6.94 (m, 2H), 6.65 (s, 1H), |
| | | 6.22-6.15 (m, 1H), 6.13 (d, J = 8.4 Hz, 1H), 4.40-4.33 (m, 3H), 4.28 (s, 1H), |
| | | 3.92-3.80 (m, 1H), 3.66-3.56 (m, 2H), 3.47 (d, J = 10.0 Hz, 2H), 3.20-3.06 |
| | | (m, 6H), 3.01-2.93 (m, 3H), 2.79-2.72 (m, 2H), 2.67-2.64 (m, 2H), 2.33 (d, |
| | | J = 1.6 Hz, 2H), 2.28-2.18 (m, 2H), 2.07-1.99 (m, 1H), 1.87 (d, J = 10.4 Hz, |
| | | 2H), 1.61-1.51 (m, 2H), 1.46-1.34 (m, 3H) |
| I-1115 | 879.4 | 12.21 (d, J = 6.8 Hz, 1H), 10.81 (s, 1H), 7.75-7.69 (m, 1H), 7.45-7.36 (m, |
| | | 1H), 7.32-7.25 (m, 1H), 7.05-6.95 (m, 5H), 6.66 (s, 1H), 6.22-6.12 (m, |
| | | 2H), 4.42-4.26 (m, 5H), 3.81 (J = 4.4, 11.6 Hz, 1H), 3.66-3.57 (m, 2H), 3.15 |
| | | (s, 6H), 3.07-2.94 (m, 9H), 2.69-2.62 (m, 1H), 2.38-2.24 (m, 3H), 2.23- |
| | | 2.14 (m, 1H), 2.05-1.98 (m, 1H), 1.69 (d, J = 1.6 Hz, 8H). |
| I-1116 | 851.2 | 12.22 (d, J = 8.0 Hz, 1H), 10.79 (s, 1H), 7.72 (d, J = 1.6 Hz, 1H), 7.48-7.35 |
| | | (m, 1H), 7.33-7.21 (m, 1H), 7.09-6.92 (m, 3H), 6.90-6.83 (m, 1H), 6.72- |
| | | 6.62 (m, 1H), 6.57-6.42 (m, 1H), 6.25-6.05 (m, 2H), 4.41-4.28 (m, 4H), |
| | | 3.78-3.72 (m, 4H), 3.68-3.61 (m, 1H), 3.60-3.56 (m, 1H), 3.21-3.03 (m, |
| | | 9H), 3.01-2.93 (m, 2H), 2.69-2.60 (m, 1H), 2.57-2.53 (m, 1H), 2.49-2.44 |
| | | (m, 1H), 2.36-2.25 (m, 2H), 2.22 (s, 1H), 2.01 (d, J = 4.0 Hz, 1H), 1.96 (s, |
| | | 4H) |
| I-1117 | 895.3 | 12.34-12.14 (m, 1H), 10.82 (s, 1H), 8.17-8.07 (m, 1H), 7.74-7.64 (m, 1H), |
| | | 7.34-7.25 (m, 1H), 7.06-6.92 (m, 5H), 6.66 (br d, J = 2.3 Hz, 1H), 6.22- |
| | | 6.11 (m, 1H), 4.69-4.55 (m, 2H), 4.42-4.28 (m, 2H), 3.80 (dd, J = 4.8, 11.6 |
| | | Hz, 1H), 3.68-3.57 (m, 2H), 3.42 (br d, J = 11.2 Hz, 2H), 3.26-2.96 (m, |
| | | 13H), 2.77-2.62 (m, 7H), 2.42-2.33 (m, 2H), 2.31-2.13 (m, 2H), 2.05-1.88 |
| | | (m, 3H), 1.70-1.53 (m, 2H) |
| I-1118 | 859.4 | 12.10 (s, 1H), 11.05 (s, 1H), 7.71 (dd, J = 1.6, 10.4 Hz, 1H), 7.46-7.35 (m, |
| | | 4H), 7.29 (t, J = 7.6 Hz, 1H), 7.19-7.12 (m, 1H), 7.06 (d, J = 5.2 Hz, 1H), |
| | | 6.96 (d, J = 6.8 Hz, 1H), 6.86 (s, 1H), 6.20 (td, J = 1.6, 10.8 Hz, 1H), 5.72 (dd, |
| | | J = 4.8, 11.6 Hz, 1H), 4.53-4.42 (m, 1H), 4.35 (q, J = 6.8 Hz, 2H), 3.89 (d, J = |
| | | 12.0 Hz, 1H), 3.24-3.00 (m, 14H), 2.95-2.80 (m, 4H), 2.71 (d, J = 10.8 |
| | | Hz, 6H), 2.66 (s, 6H), 2.28-2.16 (m, 1H), 1.91 (s, 2H), 1.76 (d, J = 8.8 Hz, |
| | | 1H) |
| I-1119 | 775.4 | 12.36-12.14 (m, 1H), 10.99-10.79 (m, 1H), 8.21-8.02 (m, 1H), 7.76 (s, |
| | | 1H), 7.62 (d, J = 8.4 Hz, 2H), 7.46-7.30 (m, 4H), 7.08-6.98 (m, 1H), 6.87 (d, |
| | | J = 2.4 Hz, 1H), 6.24-6.14 (m, 2H), 4.38-4.27 (m, 4H), 4.17-4.08 (m, 1H), |
| | | 4.02-3.96 (m, 2H), 3.69-3.57 (m, 2H), 3.23-3.11 (m, 5H), 2.99-2.90 (m, |
| | | 4H), 2.86-2.80 (m, 1H), 2.74-2.60 (m, 2H), 2.38-2.24 (m, 4H), 2.12-2.05 |
| | | (m, 1H), 1.94 (br d, J = 12.0 Hz, 2H), 1.83-1.74 (m, 2H). |
| I-1120 | 847.3 | 12.26-11.86 (m, 1H), 11.15-10.65 (m, 1H), 8.81-8.69 (m, 1H), 7.80-7.66 |
| | | (m, 1H), 7.57-7.50 (m, 2H), 7.48-7.33 (m, 1H), 7.08 (br d, J = 8.8 Hz, 2H), |
| | | 6.92 (s, 1H), 6.89-6.82 (m, 1H), 6.47 (s, 1H), 6.24-6.11 (m, 2H), 4.81-4.68 |
| | | (m, 1H), 4.41-4.29 (m, 4H), 3.74 (s, 4H), 3.68-3.57 (m, 2H), 3.28-3.20 (m, |
| | | 6H), 3.14 (br s, 3H), 3.03-2.91 (m, 3H), 2.86-2.75 (m, 1H), 2.35-2.22 (m, |
| | | 3H), 2.09-1.84 (m, 6H). |
| I-1121 | 876.4 | 12.28-12.12 (m, 1H), 10.88-10.74 (m, 1H), 7.75-7.67 (m, 1H), 7.47-7.36 |
| | | (m, 3H), 7.17-7.10 (m, 1H), 7.06-6.93 (m, 4H), 6.87 (br s, 1H), 6.22-6.13 |
| | | (m, 2H), 4.39-4.28 (m, 4H), 3.80 (br dd, J = 4.8, 11.6 Hz, 1H), 3.67-3.56 (m, |
| | | 2H), 3.41 (br d, J = 10.0 Hz, 3H), 3.19 (br s, 2H), 3.10 (br s, 6H), 3.01-2.90 |
| | | (m, 3H), 2.71 (br s, 4H), 2.69-2.60 (m, 3H), 2.44-2.30 (m, 3H), 2.26 (br s, |
| | | 1H), 2.23-2.13 (m, 1H), 2.05-1.97 (m, 1H), 1.91 (br d, J = 10.8 Hz, 2H), |
| | | 1.69-1.56 (m, 2H). |
| I-1122 | 925.2 | 12.27-12.12 (m, 1H), 10.54-10.47 (m, 1H), 7.79-7.61 (m, 4H), 7.46-7.23 |
| | | (m, 2H), 7.05-6.89 (m, 2H), 6.70-6.57 (m, 1H), 6.26-6.06 (m, 2H), 4.46- |
| | | 4.40 (m, 1H), 4.39-4.33 (m, 3H), 4.32-4.26 (m, 1H), 4.18-4.12 (m, 1H), |
| | | 4.04-3.98 (m, 1H), 3.79-3.71 (m, 2H), 3.67-3.53 (m, 4H), 3.20-3.15 (m, |
| | | 2H), 3.11 (br s, 6H), 3.07-3.01 (m, 2H), 3.00-2.90 (m, 4H), 2.78 (br s, 6H), |
| | | 2.34-2.24 (m, 2H). |
| I-1123 | 939.2 | 12.31-12.18 (m, 1H), 10.59-10.46 (m, 1H), 7.77 (d, J = 1.6 Hz, 1H), 7.74- |
| | | 7.69 (m, 1H), 7.68-7.58 (m, 2H), 7.45-7.35 (m, 1H), 7.33-7.24 (m, 1H), |
| | | 7.05-6.90 (m, 2H), 6.65 (s, 1H), 6.23-6.07 (m, 2H), 4.44-4.33 (m, 4H), |
| | | 4.32-4.26 (m, 1H), 4.15 (t, J = 9.2 Hz, 1H), 4.06-3.93 (m, 1H), 3.79-3.69 |
| | | (m, 2H), 3.68-3.54 (m, 4H), 3.48 (td, J = 2.8, 6.0 Hz, 1H), 3.19-3.02 (m, |
| | | 9H), 3.01-2.95 (m, 2H), 2.78-2.66 (m, 3H), 2.55 (s, 3H), 2.37-2.23 (m, |
| | | 4H), 2.03-1.71 (m, 2H). |
| I-1124 | 810.3 | 12.49-12.04 (m, 1H), 10.90-10.77 (m, 1H), 7.78-7.65 (m, 1H), 7.44-7.34 |
| | | (m, 3H), 7.10-6.98 (m, 4H), 6.69-6.54 (m, 1H), 6.23-6.08 (m, 2H), 4.38- |
| | | 4.34 (m, 3H), 4.31-4.26 (m, 1H), 3.93-3.84 (m, 1H), 3.65-3.56 (m, 4H), |
| | | 3.23-3.10 (m, 4H), 3.05-2.83 (m, 8H), 2.75-2.63 (m, 2H), 2.28-2.20 (m, |
| | | 2H), 2.06-2.00 (m, 1H), 1.94-1.86 (m, 4H) |
| I-1125 | 859.4 | 12.12 (d, J = 9.6 Hz, 1H), 10.84 (s, 1H), 8.58 (d, J = 8.4 Hz, 1H), 7.78-7.68 |
| | | (m, 2H), 7.58-7.47 (m, 2H), 7.46-7.36 (m, 1H), 7.10 (d, J = 8.8 Hz, 2H), |
| | | 7.01-6.91 (m, 1H), 6.89-6.82 (m, 1H), 6.81-6.68 (m, 1H), 6.24-6.07 (m, |
| | | 2H), 4.84-4.54 (m, 1H), 4.40-4.30 (m, 4H), 3.91-3.77 (m, 4H), 3.68-3.57 |
| | | (m, 2H), 3.26 (s, 6H), 3.14-3.04 (m, 3H), 3.03-2.90 (m, 3H), 2.68 (s, 2H), |
| | | 2.36-2.25 (m, 2H), 2.22-2.08 (m, 1H), 2.05-1.97 (m, 1H), 1.96-1.84 (m, |
| | | 4H). |
| I-1126 | 917.3 | 12.74-11.24 (m, 1H), 10.83-10.01 (m, 1H), 7.78-7.65 (m, 1H), 7.57-7.48 |
| | | (m, 2H), 7.46-7.34 (m, 1H), 7.28 (d, J = 9.2 Hz, 1H), 7.05 (br d, J = 8.8 Hz, |
| | | 2H), 7.00-6.83 (m, 3H), 6.78 (dd, J = 2.8, 9.2 Hz, 1H), 6.24-6.11 (m, 2H), |
| | | 4.42-4.29 (m, 4H), 3.86-3.52 (m, 8H), 3.24-2.92 (m, 10H), 2.78-2.67 (m, |
| | | 7H), 2.36-2.18 (m, 4H), 2.10-2.00 (m, 2H), 1.87-1.67 (m, 5H), 1.60 (br d, J = |
| | | 10.2 Hz, 2H), 1.29-1.19 (m, 2H) |
| I-1127 | 904.3 | 12.02 (br s, 1H), 10.52 (s, 1H), 7.76-7.63 (m, 2H), 7.57 (d, J = 2.0 Hz, 1H), |
| | | 7.50 (d, J = 8.8 Hz, 2H), 7.45-7.37 (m, 2H), 7.09-6.97 (m, 3H), 6.84 (br s, |
| | | 1H), 6.20 (td, J = 2.0, 10.4 Hz, 1H), 4.49 (br d, J = 11.6 Hz, 2H), 4.41-4.32 |
| | | (m, 2H), 3.92-3.75 (m, 4H), 3.63 (br dd, J = 5.6, 11.6 Hz, 2H), 3.20-3.01 |
| | | (m, 7H), 2.95-2.88 (m, 2H), 2.81-2.62 (m, 6H), 2.00-1.62 (m, 8H), 1.54- |
| | | 1.10 (m, 8H) |
| I-1128 | 891.3 | 12.16-12.06 (m, 1H), 10.45 (br s, 1H), 7.74-7.69 (m, 1H), 7.50 (br d, J = 8.4 |
| | | Hz, 2H), 7.44-7.37 (m, 2H), 7.14 (d, J = 2.8 Hz, 1H), 7.05 (br d, J = 8.8 Hz, |
| | | 2H), 7.00-6.91 (m, 2H), 6.85 (br s, 1H), 6.21-6.12 (m, 2H), 4.39-4.34 (m, |
| | | 4H), 4.31-4.27 (m, 1H), 3.85-3.79 (m, 2H), 3.72-3.63 (m, 2H), 3.60-3.56 |
| | | (m, 2H), 3.21-3.04 (m, 6H), 3.01-2.93 (m, 3H), 2.84-2.78 (m, 2H), 2.76- |
| | | 2.67 (m, 5H), 2.37-2.30 (m, 2H), 2.29-2.23 (m, 1H), 1.99-1.92 (m, 2H), |
| | | 1.88-1.80 (m, 2H), 1.63-1.53 (m, 4H) |
| I-1129 | 806.3 | 12.40-11.97 (m, 1H), 11.13-10.86 (m, 1H), 8.29-8.13 (m, 1H), 7.80-7.70 |
| | | (m, 2H), 7.69-7.63 (m, 2H), 7.59-7.41 (m, 5H), 7.40-7.37 (m, 1H), 7.23 (d, |
| | | J = 7.9 Hz, 1H), 7.11-7.01 (m, 1H), 6.90 (d, J = 2.9 Hz, 1H), 6.28-6.09 (m, |
| | | 2H), 4.75-4.63 (m, 1H), 4.43-4.32 (m, 4H), 3.70-3.57 (m, 2H), 3.51-3.40 |
| | | (m, 2H), 3.28-3.07 (m, 5H), 3.01-2.90 (m, 4H), 2.88-2.78 (m, 2H), 2.41- |
| | | 2.27 (m, 4H), 2.21-1.96 (m, 6H) |
| I-1130 | 893.2 | 12.27-12.17 (m, 1H), 10.89-10.76 (m, 1H), 7.77-7.67 (m, 1H), 7.51-7.33 |
| | | (m, 1H), 7.32-7.22 (m, 1H), 7.03-6.93 (m, 5H), 6.70-6.60 (m, 1H), 6.22- |
| | | 6.09 (m, 2H), 4.38-4.27 (m, 4H), 3.83-3.77 (m, 1H), 3.67-3.61 (m, 1H), |
| | | 3.60-3.50 (m, 4H), 3.20-3.15 (m, 2H), 3.01-2.93 (m, 3H), 2.81-2.68 (m, |
| | | 3H), 2.68-2.56 (m, 5H), 2.35-2.15 (m, 4H), 2.03-1.96 (m, 1H), 1.88-1.79 |
| | | (m, 4H), 1.47-1.21 (m, 7H) |
| I-1131 | 809.5 | 12.32-12.14 (m, 1H), 10.60-10.49 (m, 1H), 9.60-9.47 (m, 1H), 8.59-8.50 |
| | | (m, 1H), 8.17-8.08 (m, 1H), 7.80-7.60 (m, 5H), 7.54 (d, J = 8.2 Hz, 2H), |
| | | 7.38-7.31 (m, 1H), 7.11 (d, J = 6.1 Hz, 1H), 6.95-6.87 (m, 1H), 6.27-6.14 |
| | | (m, 1H), 4.70-4.60 (m, 2H), 4.46-4.33 (m, 2H), 3.99-3.86 (m, 1H), 3.78- |
| | | 3.64 (m, 2H), 3.63-3.50 (m, 3H), 3.26-3.14 (m, 3H), 3.14-2.93 (m, 8H), |
| | | 2.90-2.82 (m, 1H), 2.81-2.73 (m, 1H), 2.41-2.35 (m, 1H), 2.34-2.25 (m, |
| | | 1H), 2.11 (br s, 2H), 2.09-2.00 (m, 2H) |
| I-1132 | 802.7 | 1.43 (br d, J = 11.6 Hz, 2 H) 1.63 (br s, 2 H) 1.75 (br d, J = 7.8 Hz, 4 H) 1.84- |
| | | 2.02 (m, 4 H) 2.15-2.36 (m, 3 H) 2.60-2.75 (m, 5 H) 2.78-3.25 (m, 14 H) |
| | | 3.50-3.72 (m, 2 H) 4.21-4.49 (m, 4 H) 5.68 (dd, J = 11.8, 5.2 Hz, 1 H) 6.06 |
| | | (br s, 1 H) 6.15-6.24 (m, 1 H) 6.67 (d, J = 7.6 Hz, 1 H) 6.78-6.88 (m, 1 H) |
| | | 6.94 (br s, 1 H) 7.12-7.18 (m, 1 H) 7.18-7.28 (m, 1 H) 7.33-7.50 (m, 1 H) |
| | | 7.67-7.79 (m, 1 H) 10.90-11.21 (m, 1 H) 11.94 (br s, 1 H) |
| I-1133 | 808.4 | 12.19 (br d, J = 2.8 Hz, 1H), 10.54 (s, 1H), 9.51 (s, 1H), 8.54 (s, 1H), 7.77- |
| | | 7.70 (m, 2H), 7.67-7.61 (m, 3H), 7.53 (d, J = 8.0 Hz, 2H), 7.45-7.37 (m, |
| | | 1H), 7.33 (d, J = 7.6 Hz, 1H), 7.10-7.01 (m, 1H), 6.90 (d, J = 2.8 Hz, 1H), |
| | | 6.24-6.11 (m, 2H), 4.41-4.32 (m, 4H), 3.92 (ddd, J = 5.2, 9.8, 12.2 Hz, 1H), |
| | | 3.75-3.49 (m, 6H), 3.23-3.16 (m, 2H), 3.09-2.92 (m, 8H), 2.88-2.81 (m, |
| | | 1H), 2.79-2.73 (m, 1H), 2.37-2.27 (m, 2H), 2.20-2.10 (m, 2H), 2.07-2.01 |
| | | (m, 2H) |
| I-1134 | 930.8 | 12.40-12.18 (m, 1H), 10.95-10.77 (m, 1H), 8.21-8.03 (m, 1H), 7.79-7.59 |
| | | (m, 1H), 7.48-7.37 (m, 2H), 7.14-6.94 (m, 4H), 6.73-6.65 (m, 1H), 6.24- |
| | | 6.13 (m, 1H), 4.66-4.60 (m, 2H), 4.41-4.33 (m, 2H), 3.84-3.76 (m, 1H), |
| | | 3.71-3.55 (m, 3H), 3.43 (br d, J = 10.0 Hz, 3H), 3.25-3.18 (m, 2H), 3.14- |
| | | 3.01 (m, 7H), 2.83-2.59 (m, 6H), 2.45-2.24 (m, 3H), 2.23-2.11 (m, 2H), |
| | | 2.07-1.99 (m, 1H), 1.92-1.82 (m, 3H), 1.72-1.61 (m, 2H) |
| I-1135 | 894.3 | 12.23 (d, J = 6.0 Hz, 1H), 10.82 (s, 1H), 8.17-8.07 (m, 1H), 7.78-7.58 (m, |
| | | 1H), 7.35-7.22 (m, 1H), 7.05-6.92 (m, 5H), 6.66 (d, J = 1.6 Hz, 1H), 6.14 (s, |
| | | 1H), 4.62 (d, J = 8.8 Hz, 2H), 4.43-4.29 (m, 2H), 3.80 (dd, J = 4.8, 11.6 Hz, |
| | | 1H), 3.66 (s, 1H), 3.62-3.59 (m, 1H), 3.53 (d, J = 11.2 Hz, 2H), 3.40 (d, J = |
| | | 10.4 Hz, 2H), 3.25-3.14 (m, 3H), 3.13-2.99 (m, 6H), 2.74 (s, 1H), 2.61 (s, |
| | | 2H), 2.39-2.26 (m, 3H), 2.20 (dd, J = 3.2, 12.0 Hz, 1H), 2.03-1.95 (m, 1H), |
| | | 1.84 (s, 5H), 1.45-1.35 (m, 4H), 1.30 (s, 2H) |
| I-1136 | 793.3 | δ = 12.30 (s, 1H), 10.83 (s, 1H), 8.57 (s, 1H), 7.94 (d, J = 3.3 Hz, 1H), 7.76- |
| | | 7.68 (m, 1H), 7.47-7.28 (m, 2H), 7.10-6.90 (m, 4H), 6.27-6.06 (m, 2H), |
| | | 4.44-4.23 (m, 4H), 3.83 (dd, J = 4.8, 11.6 Hz, 1H), 3.70 (s, 3H), 3.22-3.07 |
| | | (m, 5H), 2.99-2.92 (m, 2H), 2.90-2.82 (m, 2H), 2.78-2.55 (m, 1H), 2.39- |
| | | 2.30 (m, 3H), 2.28-2.21 (m, 2H), 2.08-1.85 (m, 7H) |
| I-1137 | 807.2 | 12.22-12.13 (m, 1H), 10.97 (br s, 1H), 9.50 (s, 1H), 8.39 (s, 1H), 7.74-7.69 |
| | | (m, 2H), 7.67-7.64 (m, 3H), 7.53 (br d, J = 8.0 Hz, 2H), 7.44-7.37 (m, 1H), |
| | | 7.30 (br d, J = 7.2 Hz, 1H), 7.06 (br dd, J = 6.4, 19.6 Hz, 1H), 6.89 (br s, 1H), |
| | | 6.19 (br d, J = 12.4 Hz, 2H), 4.63-4.57 (m, 1H), 4.41-4.30 (m, 5H), 3.67- |
| | | 3.58 (m, 2H), 3.55-3.51 (m, 2H), 3.04-2.96 (m, 6H), 2.90-2.79 (m, 3H), |
| | | 2.68-2.59 (m, 2H), 2.35-2.26 (m, 3H), 2.19-2.08 (m, 4H), 2.06-2.01 (m, |
| | | 2H) |
| I-1138 | 864.0 | 12.24 (br d, J = 8.1 Hz, 1H), 11.12-11.02 (m, 1H), 7.76-7.65 (m, 2H), 7.45- |
| | | 7.33 (m, 2H), 7.29-7.24 (m, 1H), 7.18 (s, 1H), 7.05-6.95 (m, 1H), 6.72- |
| | | 6.65 (m, 1H), 6.56-6.48 (m, 1H), 6.24-6.13 (m, 2H), 5.75-5.64 (m, 1H), |
| | | 4.39-4.28 (m, 4H), 3.69-3.56 (m, 2H), 3.48 (br d, J = 9.8 Hz, 2H), 3.06 (br s, |
| | | 6H), 3.04-2.93 (m, 4H), 2.92-2.82 (m, 3H), 2.77-2.72 (m, 1H), 2.69 (s, |
| | | 3H), 2.33 (br s, 1H), 2.28-2.19 (m, 2H), 2.13-2.04 (m, 2H), 2.00 (br s, 2H) |
| I-1139 | 863.4 | 12.33-12.17 (m, 1H), 11.12-10.99 (m, 1H), 8.46-8.42 (m, 1H), 8.18-8.05 |
| | | (m, 1H), 7.76-7.66 (m, 2H), 7.36 (s, 1H), 7.30-7.25 (m, 1H), 7.21-7.17 (m, |
| | | 1H), 7.07-6.94 (m, 1H), 6.74 (s, 1H), 6.61-6.45 (m, 1H), 6.23-6.09 (m, |
| | | 1H), 5.77-5.65 (m, 1H), 4.67-4.59 (m, 2H), 4.42-4.32 (m, 2H), 3.67-3.58 |
| | | (m, 2H), 3.53-3.46 (m, 2H), 3.21-2.99 (m, 10H), 2.94-2.83 (m, 3H), 2.75- |
| | | 2.72 (m, 1H), 2.69 (s, 3H), 2.35-2.33 (m, 1H), 2.28-2.19 (m, 2H), 2.12- |
| | | 2.04 (m, 2H), 2.03-1.97 (m, 2H) |
| I-1140 | 802.5 | 10.96-10.58 (m, 1H), 8.11 (d, J = 10.8 Hz, 1H), 7.79 (d, J = 8.4 Hz, 2H), 7.69 |
| | | (d, J = 10.8 Hz, 1H), 7.46 (d, J = 8.4 Hz, 2H), 7.42-7.28 (m, 1H), 7.10-6.93 |
| | | (m, 4H), 6.20-6.11 (m, 1H), 4.66-4.60 (m, 2H), 4.36-4.31 (m, 2H), 3.92 (s, |
| | | 2H), 3.84-3.81 (m, 2H), 3.66 (s, 2H), 3.61-3.59 (m, 2H), 3.50 (s, 2H), 3.47 |
| | | (s, 2H), 3.12 (s, 1H), 3.10 (d, J = 7.2 Hz, 2H), 2.84-2.82 (m, 2H), 2.79 (s, |
| | | 1H), 2.38-2.33 (m, 2H), 2.29-2.23 (m, 2H), 1.93-1.84 (m, 6H) |
| I-1141 | 930.4 | 12.18 (d, J = 2.0 Hz, 1H), 10.81 (s, 1H), 7.76-7.66 (m, 1H), 7.45-7.30 (m, |
| | | 2H), 7.09-6.96 (m, 5H), 6.63 (d, J = 2.4 Hz, 1H), 6.24-6.09 (m, 2H), 4.39- |
| | | 4.28 (m, 4H), 3.82 (dd, J = 4.8, 12.0 Hz, 1H), 3.66-3.56 (m, 2H), 3.55-3.45 |
| | | (m, 2H), 3.20-3.02 (m, 12H), 3.00-2.85 (m, 8H), 2.71-2.61 (m, 1H), 2.34- |
| | | 2.16 (m, 3H), 2.06-1.91 (m, 3H) |
| I-1142 | 930.4 | 12.23-12.14 (m, 1H), 10.81 (s, 1H), 7.75-7.67 (m, 1H), 7.45-7.31 (m, 2H), |
| | | 7.09-6.96 (m, 5H), 6.63 (s, 1H), 6.23-6.10 (m, 2H), 4.39-4.28 (m, 4H), |
| | | 3.82 (dd, J = 4.8, 11.6 Hz, 1H), 3.66-3.56 (m, 2H), 3.55-3.45 (m, 2H), 3.19- |
| | | 3.02 (m, 12H), 3.00-2.85 (m, 8H), 2.65 (dt, J = 5.6, 11.6 Hz, 1H), 2.34-2.18 |
| | | (m, 3H), 2.05-1.92 (m, 3H) |
| I-1143 | 912.4 | 12.20 (d, J = 6.4 Hz, 1H), 10.88 (s, 1H), 7.76-7.66 (m, 1H), 7.47-7.29 (m, |
| | | 2H), 7.06-6.94 (m, 2H), 6.65 (d, J = 12.4 Hz, 3H), 6.25-6.10 (m, 2H), 4.37 |
| | | (d, J = 6.8 Hz, 4H), 4.07 (dd, J = 5.2, 12.4 Hz, 1H), 3.71-3.44 (m, 5H), 3.21 |
| | | (s, 6H), 3.12-2.93 (m, 6H), 2.78 (d, J = 12.0 Hz, 3H), 2.65 (s, 4H), 2.46-2.39 |
| | | (m, 1H), 2.36-2.24 (m, 2H), 2.17-2.03 (m, 1H), 2.00-1.88 (m, 3H), 1.62 (d, |
| | | J = 9.6 Hz, 2H) |
| I-1144 | 907.4 | 12.19 (d, J = 8.0 Hz, 1H), 10.85 (s, 1H), 7.75-7.66 (m, 1H), 7.46-7.29 (m, |
| | | 2H), 7.09-6.94 (m, 4H), 6.63 (s, 1H), 6.24-6.08 (m, 2H), 4.39-4.26 (m, |
| | | 4H), 3.94 (dd, J = 4.8, 12.4 Hz, 1H), 3.66-3.50 (m, 5H), 3.20-3.12 (m, 3H), |
| | | 3.06-2.92 (m, 6H), 2.84-2.58 (m, 5H), 2.54 (d, J = 3.6 Hz, 1H), 2.33 (s, 4H), |
| | | 2.26 (s, 3H), 2.17 (dd, J = 3.6, 12.8 Hz, 1H), 1.89 (d, J = 11.2 Hz, 3H), 1.74- |
| | | 1.59 (m, 6H) |
| I-1145 | 839.8 | 1.55-1.73 (m, 4 H) 1.80-1.91 (m, 4 H) 1.96-2.06 (m, 1 H) 2.07-2.20 (m, 1 |
| | | H) 2.26-2.36 (m, 4 H) 2.39-2.44 (m, 1 H) 2.56-2.72 (m, 4 H) 2.92-3.26 |
| | | (m, 11 H) 3.55-3.68 (m, 2 H) 3.69-3.78 (m, 3 H) 4.30-4.40 (m, 4 H) 6.12- |
| | | 6.23 (m, 2 H) 6.84-6.95 (m, 3 H) 6.98-7.09 (m, 3 H) 7.34-7.46 (m, 3 H) |
| | | 7.59 (br d, J = 7.6 Hz, 2 H) 7.69-7.76 (m, 1 H) 10.78 (s, 1 H) 12.18 (br dd, |
| | | J = 5.6, 2.2 Hz, 1 H) |
| I-1146 | 930.2 | 12.31-12.13 (m, 1H), 10.88-10.76 (m, 1H), 8.47-8.41 (m, 1H), 7.74-7.68 |
| | | (m, 1H), 7.46-7.37 (m, 1H), 7.37-7.24 (m, 1H), 7.08-6.94 (m, 5H), 6.71- |
| | | 6.60 (m, 1H), 6.23-6.13 (m, 2H), 4.34 (br s, 4H), 3.86-3.77 (m, 1H), 3.68- |
| | | 3.56 (m, 4H), 3.27 (br d, J = 6.4 Hz, 1H), 3.23-3.12 (m, 4H), 3.11-2.92 (m, |
| | | 15H), 2.69-2.63 (m, 1H), 2.35-2.32 (m, 1H), 2.31-2.15 (m, 2H), 2.14-1.79 |
| | | (m, 3H) |
| I-1147 | 930.2 | 12.46-11.90 (m, 1H), 11.02-10.74 (m, 1H), 8.35 (s, 1H), 7.76-7.71 (m, |
| | | 1H), 7.44-7.38 (m, 1H), 7.33-7.25 (m, 1H), 7.06-6.94 (m, 5H), 6.70-6.63 |
| | | (m, 1H), 6.24-6.13 (m, 2H), 4.39-4.30 (m, 4H), 3.86-3.78 (m, 1H), 3.68- |
| | | 3.56 (m, 4H), 3.28-3.16 (m, 5H), 3.09-2.91 (m, 15H), 2.72-2.64 (m, 1H), |
| | | 2.35-2.32 (m, 1H), 2.17 (br s, 2H), 2.07-1.96 (m, 3H) |
| I-1148 | 810.3 | 12.22 (d, J = 7.6 Hz, 1H), 10.95-10.73 (m, 1H), 7.75-7.64 (m, 3H), 7.53- |
| | | 7.48 (m, 2H), 7.45-7.37 (m, 1H), 7.10 (d, J = 3.6 Hz, 2H), 7.06-7.01 (m, |
| | | 1H), 6.92-6.87 (m, 1H), 6.24-6.15 (m, 2H), 4.41-4.31 (m, 4H), 3.87-3.83 |
| | | (m, 1H), 3.71-3.64 (m, 2H), 3.58 (d, J = 5.6 Hz, 2H), 3.24-3.07 (m, 6H), |
| | | 3.02-2.96 (m, 3H), 2.70-2.63 (m, 2H), 2.55-2.54 (m, 1H), 2.34 (s, 3H), |
| | | 2.30-2.18 (m, 2H), 2.08-1.96 (m, 2H) |
| I-1149 | 893.9 | 12.32-11.95 (m, 1H), 11.01-10.71 (m, 1H), 7.79-7.64 (m, 1H), 7.46-7.28 |
| | | (m, 2H), 7.24 (t, J = 7.9 Hz, 1H), 7.13-7.05 (m, 2H), 7.04-6.95 (m, 2H), 6.64 |
| | | (d, J = 2.3 Hz, 1H), 6.26-6.08 (m, 2H), 4.41-4.35 (m, 3H), 4.28 (s, 1H), 4.08- |
| | | 3.94 (m, 1H), 3.68-3.61 (m, 1H), 3.60-3.52 (m, 3H), 3.30-3.05 (m, 6H), |
| | | 3.05-3.00 (m, 3H), 2.99-2.92 (m, 2H), 2.79-2.67 (m, 3H), 2.59-2.54 (m, |
| | | 1H), 2.47-2.41 (m, 1H), 2.32 (d, J = 3.9 Hz, 2H), 2.28 (d, J = 10.0 Hz, 2H), |
| | | 2.23-2.13 (m, 1H), 2.05-1.97 (m, 1H), 1.93-1.85 (m, 2H), 1.80 (d, J = 11.5 |
| | | Hz, 2H), 1.70-1.59 (m, 4H) |
| I-1150 | 909.6 | 12.22 (d, J = 6.8 Hz, 1H), 10.81 (s, 1H), 8.16-8.05 (m, 1H), 7.74-7.63 (m, |
| | | 1H), 7.46-7.19 (m, 1H), 7.06-6.91 (m, 5H), 6.74-6.47 (m, 1H), 6.19-6.11 |
| | | (m, 1H), 4.67-4.57 (m, 2H), 4.41-4.28 (m, 2H), 3.85-3.75 (m, 1H), 3.67- |
| | | 3.57 (m, 2H), 3.54-3.47 (m, 2H), 3.43-3.39 (m, 2H), 3.18-2.99 (m, 9H), |
| | | 2.61 (s, 8H), 2.38 (s, 2H), 2.30-2.23 (m, 1H), 2.15 (s, 1H), 2.05-1.97 (m, |
| | | 1H), 1.95-1.86 (m, 2H), 1.69-1.56 (m, 2H), 1.18-1.09 (m, 3H) |
| I-1151 | 929.9 | 12.27-12.17 (m, 1H), 10.92-10.85 (m, 1H), 7.76-7.69 (m, 1H), 7.38 (s, |
| | | 1H), 7.33-7.26 (m, 1H), 7.10-7.03 (m, 2H), 7.01-6.92 (m, 2H), 6.68-6.63 |
| | | (m, 1H), 6.18 (s, 1H), 6.16-6.11 (m, 1H), 4.39-4.35 (m, 3H), 4.33-4.28 (m, |
| | | 1H), 4.19-4.13 (m, 1H), 3.71-3.61 (m, 1H), 3.58 (br t, J = 5.6 Hz, 1H), 3.47- |
| | | 3.41 (m, 2H), 3.17-3.11 (m, 6H), 3.08-3.02 (m, 2H), 3.01-2.93 (m, 3H), |
| | | 2.80-2.77 (m, 1H), 2.74 (br s, 6H), 2.57-2.54 (m, 1H), 2.44-2.40 (m, 2H), |
| | | 2.35-2.31 (m, 2H), 2.29-2.26 (m, 1H), 1.99-1.89 (m, 3H), 1.68-1.58 (m, |
| | | 2H) |
| I-1152 | 930.4 | 12.25 (br d, J = 8.6 Hz, 1H), 10.82 (s, 1H), 7.71 (dd, J = 2.0, 11.5 Hz, 1H), |
| | | 7.43 (d, J = 1.3 Hz, 1H), 7.45-7.26 (m, 1H), 7.09-6.95 (m, 4H), 6.69-6.62 |
| | | (m, 1H), 6.22-6.11 (m, 2H), 4.39-4.28 (m, 4H), 3.85-3.78 (m, 1H), 3.65- |
| | | 3.46 (m, 4H), 3.19-3.04 (m, 8H), 3.00-2.95 (m, 2H), 2.92-2.84 (m, 2H), |
| | | 2.73 (br d, J = 13.6 Hz, 2H), 2.61 (br s, 1H), 2.55-2.53 (m, 3H), 2.19 (br s, |
| | | 3H), 2.05-1.89 (m, 3H), 1.84-1.71 (m, 4H) |
| I-1153 | 912.5 | 12.30-11.99 (m, 1H), 10.86 (s, 1H), 7.74-7.68 (m, 1H), 7.45-7.30 (m, 2H), |
| | | 7.18-7.07 (m, 1H), 7.05-6.94 (m, 2H), 6.90-6.81 (m, 1H), 6.63 (d, J = 1.6 |
| | | Hz, 1H), 6.24-6.07 (m, 2H), 4.38-4.28 (m, 4H), 4.02-3.90 (m, 1H), 3.64 (t, |
| | | J = 5.6 Hz, 1H), 3.59-3.52 (m, 3H), 3.25-3.08 (m, 6H), 3.05 (d, J = 1.2 Hz, |
| | | 4H), 3.01-2.93 (m, 3H), 2.81-2.72 (m, 3H), 2.71-2.65 (m, 5H), 2.54 (d, J = |
| | | 4.0 Hz, 2H), 2.29-2.17 (m, 2H), 1.96-1.90 (m, 2H), 1.69-1.56 (m, 2H) |
| I-1154 | 907.6 | 12.41-12.30 (m, 1H), 11.03 (d, J = 7.4 Hz, 1H), 8.00-7.76 (m, 1H), 7.61- |
| | | 7.46 (m, 2H), 7.27-7.08 (m, 4H), 6.85-6.69 (m, 1H), 6.43-6.22 (m, 2H), |
| | | 4.56-4.45 (m, 4H), 4.20-4.11 (m, 1H), 3.81 (t, J = 5.6 Hz, 1H), 3.77-3.66 |
| | | (m, 4H), 3.43-3.28 (m, 6H), 3.25-3.09 (m, 6H), 2.98-2.83 (m, 5H), 2.53- |
| | | 2.42 (m, 4H), 2.38-2.29 (m, 4H), 2.19-2.02 (m, 3H), 1.93-1.76 (m, 4H) |
| I-1155 | 911.6 | 12.20 (d, J = 8.0 Hz, 1H), 10.91 (s, 1H), 7.77-7.65 (m, 1H), 7.47-7.29 (m, |
| | | 2H), 7.25-7.13 (m, 2H), 7.06-6.93 (m, 2H), 6.64 (s, 1H), 6.27-6.06 (m, |
| | | 2H), 4.40-4.28 (m, 4H), 4.07-4.01 (m, 1H), 3.65 (t, J = 4.8 Hz, 1H), 3.60- |
| | | 3.52 (m, 3H), 3.26-3.10 (m, 6H), 3.07-2.95 (m, 6H), 2.82-2.70 (m, 4H), |
| | | 2.32 (d, J = 9.2 Hz, 3H), 2.29-2.17 (m, 2H), 2.05-1.95 (m, 1H), 1.89 (d, J = |
| | | 12.4 Hz, 2H), 1.78-1.62 (m, 6H) |
| I-1156 | 908.5 | 12.27-12.16 (m, 1H), 10.83-10.78 (m, 1H), 7.74-7.66 (m, 1H), 7.53-7.34 |
| | | (m, 1H), 7.33-7.25 (m, 1H), 7.04-6.93 (m, 5H), 6.69-6.53 (m, 1H), 6.21- |
| | | 6.11 (m, 2H), 4.36 (d, J = 7.2 Hz, 3H), 4.31-4.25 (m, 1H), 3.82-3.77 (m, |
| | | 1H), 3.67-3.61 (m, 1H), 3.57 (t, J = 5.6 Hz, 1H), 3.51 (d, J = 6.0 Hz, 2H), |
| | | 3.41 (d, J = 10.8 Hz, 3H), 3.16-3.11 (m, 5H), 3.02-2.93 (m, 3H), 2.75-2.61 |
| | | (m, 8H), 2.35-2.14 (m, 4H), 2.03-1.97 (m, 1H), 1.94-1.87 (m, 2H), 1.67- |
| | | 1.57 (m, 2H), 1.17-1.11 (m, 3H) |
| I-1157 | 930.6 | 12.30-12.16 (m, 1H), 10.89-10.78 (m, 1H), 8.50-8.47 (m, 1H), 7.76-7.69 |
| | | (m, 1H), 7.46-7.35 (m, 2H), 7.12-6.96 (m, 5H), 6.68-6.61 (m, 1H), 6.24- |
| | | 6.11 (m, 2H), 4.29 (br s, 4H), 3.81 (dd, J = 4.8, 11.9 Hz, 1H), 3.70-3.56 (m, |
| | | 4H), 3.26-3.10 (m, 6H), 3.07-2.91 (m, 14H), 2.71-2.65 (m, 1H), 2.37-2.33 |
| | | (m, 1H), 2.17 (br s, 2H), 2.06-1.94 (m, 3H) |
| I-1158 | 930.4 | 12.27-12.11 (m, 1H), 10.84-10.79 (m, 1H), 8.52-8.49 (m, 1H), 7.78-7.68 |
| | | (m, 1H), 7.47-7.34 (m, 2H), 7.11-6.97 (m, 5H), 6.69-6.61 (m, 1H), 6.25- |
| | | 6.12 (m, 2H), 4.41-4.29 (m, 4H), 3.83-3.78 (m, 1H), 3.69-3.55 (m, 4H), |
| | | 3.26-3.10 (m, 6H), 3.06-2.91 (m, 14H), 2.71-2.64 (m, 1H), 2.35-2.32 (m, |
| | | 1H), 2.29-2.15 (m, 2H), 2.05-1.94 (m, 3H) |
| I-1159 | 840.5 | 12.31-12.08 (m, 1H), 11.07 (s, 1H), 8.16-8.08 (m, 1H), 7.69 (d, J = 12.0 Hz, |
| | | 1H), 7.65 (d, J = 8.0 Hz, 2H), 7.46 (d, J = 8.0 Hz, 2H), 7.31-7.25 (m, 1H), |
| | | 7.13 (d, J = 8.0 Hz, 1H), 7.10-7.00 (m, 1H), 6.91-6.80 (m, 2H), 6.17 (s, 1H), |
| | | 5.33-5.13 (m, 1H), 4.68-4.59 (m, 2H), 4.43-4.32 (m, 2H), 3.71-3.58 (m, |
| | | 2H), 3.25-3.05 (m, 7H), 3.03-2.92 (m, 5H), 2.90-2.74 (m, 2H), 2.70-2.57 |
| | | (m, 2H), 2.39-2.26 (m, 2H), 2.00-1.83 (m, 5H), 1.47 (d, J = 2.8 Hz, 6H) |
| I-1160 | 839.5 | 12.31-12.08 (m, 1H), 11.07 (s, 1H), 7.78-7.70 (m, 1H), 7.69-7.58 (m, 2H), |
| | | 7.53-7.36 (m, 3H), 7.33-7.22 (m, 1H), 7.13 (d, J = 8.0 Hz, 1H), 7.09-6.99 |
| | | (m, 1H), 6.93-6.79 (m, 2H), 6.29-6.08 (m, 2H), 5.35-5.11 (m, 1H), 4.44- |
| | | 4.29 (m, 4H), 3.71-3.55 (m, 2H), 3.24-3.06 (m, 5H), 3.04-2.92 (m, 7H), |
| | | 2.89-2.74 (m, 2H), 2.69-2.58 (m, 2H), 2.38-2.25 (m, 2H), 2.02-1.83 (m, |
| | | 5H), 1.47 (d, J = 2.4 Hz, 6H) |
| I-1161 | 943.6 | 12.46-12.10 (m, 1H), 11.01-10.67 (m, 1H), 7.82-7.63 (m, 1H), 7.54-7.35 |
| | | (m, 3H), 7.13-6.91 (m, 4H), 6.79-6.46 (m, 1H), 6.31-6.07 (m, 2H), 4.41- |
| | | 4.30 (m, 4H), 3.81 (br dd, J = 4.8, 11.2 Hz, 1H), 3.67-3.62 (m, 1H), 3.60- |
| | | 3.55 (m, 2H), 3.52 (br s, 2H), 3.47-3.39 (m, 4H), 3.20-3.04 (m, 4H), 3.01- |
| | | 2.96 (m, 2H), 2.75-2.65 (m, 5H), 2.39-2.26 (m, 3H), 2.24-2.12 (m, 2H), |
| | | 2.05-1.98 (m, 1H), 1.95-1.84 (m, 3H), 1.73-1.61 (m, 2H), 1.14 (br t, J = 6.8 |
| | | Hz, 3H) |
| I-1162 | 794.4 | 2.26-12.15 (m, 1H), 10.82 (s, 1H), 8.57 (s, 1H), 8.13-8.08 (m, 1H), 7.90- |
| | | 7.83 (m, 1H), 7.71-7.66 (m, 1H), 7.36-7.29 (m, 1H), 7.10-7.03 (m, 2H), |
| | | 7.01-6.97 (m, 1H), 6.94-6.88 (m, 1H), 6.19-6.12 (m, 1H), 4.66-4.60 (m, |
| | | 2H), 4.40-4.32 (m, 2H), 3.82 (dd, J = 4.8, 11.6 Hz, 1H), 3.68-3.59 (m, 2H), |
| | | 3.50 (br d, J = 11.6 Hz, 2H), 3.18 (br dd, J = 5.5, 7.8 Hz, 2H), 3.13-3.03 (m, |
| | | 5H), 2.92-2.86 (m, 1H), 2.86-2.79 (m, 2H), 2.68-2.63 (m, 1H), 2.58-2.52 |
| | | (m, 2H), 2.38-2.28 (m, 2H), 2.16 (br s, 1H), 2.06-2.00 (m, 1H), 2.00-1.91 |
| | | (m, 4H) |
| I-1163 | 929.4 | 12.51-12.08 (m, 1H), 10.93-10.62 (m, 1H), 7.71 (dd, J = 2.0, 11.6 Hz, 1H), |
| | | 7.43 (d, J = 1.3 Hz, 3H), 7.09-6.95 (m, 4H), 6.67-6.62 (m, 1H), 6.28-6.00 |
| | | (m, 2H), 4.40-4.29 (m, 4H), 3.82 (dd, J = 4.8, 11.9 Hz, 1H), 3.66-3.46 (m, |
| | | 4H), 3.21-2.95 (m, 1H), 3.23-2.81 (m, 14H), 2.77-2.61 (m, 3H), 2.34-2.17 |
| | | (m, 3H), 2.06-1.89 (m, 3H), 1.83-1.67 (m, 4H) |
| I-1164 | 930.4 | 13.18-11.34 (m, 1H), 11.04-10.61 (m, 1H), 8.15-8.06 (m, 1H), 7.75-7.60 |
| | | (m, 1H), 7.38-7.24 (m, 2H), 7.10-6.95 (m, 4H), 6.68-6.62 (m, 1H), 6.16 (d, |
| | | J = 10.0 Hz, 1H), 4.66-4.57 (m, 2H), 4.39-4.27 (m, 2H), 3.87-3.77 (m, 1H), |
| | | 3.66-3.57 (m, 2H), 3.55 (s, 2H), 3.20-2.98 (m, 12H), 2.93-2.84 (m, 2H), |
| | | 2.78-2.70 (m, 2H), 2.68-2.61 (m, 1H), 2.49-2.44 (m, 1H), 2.35 (s, 1H), |
| | | 2.27 (s, 1H), 2.24-2.15 (m, 1H), 2.07-1.96 (m, 2H), 1.91 (d, J = 12.2 Hz, |
| | | 1H), 1.85-1.70 (m, 4H) |
| I-1165 | 930.3 | 12.42-11.94 (m, 1H), 10.82 (s, 1H), 8.19-8.04 (m, 1H), 7.73-7.58 (m, 1H), |
| | | 7.43-7.22 (m, 2H), 7.13-6.92 (m, 4H), 6.71-6.58 (m, 1H), 6.26-6.03 (m, |
| | | 1H), 4.67-4.56 (m, 2H), 4.41-4.22 (m, 2H), 3.82 (dd, J = 4.8, 11.8 Hz, 1H), |
| | | 3.68-3.57 (m, 2H), 3.56-3.43 (m, 2H), 3.27-2.99 (m, 12H), 2.94-2.82 (m, |
| | | 2H), 2.79-2.70 (m, 2H), 2.67-2.59 (m, 1H), 2.49-2.44 (m, 1H), 2.35 (d, J = |
| | | 3.2 Hz, 1H), 2.27 (s, 1H), 2.23 (s, 1H), 2.08-1.96 (m, 2H), 1.95-1.87 (m, |
| | | 1H), 1.85-1.68 (m, 4H) |
| I-1166 | 894.5 | 12.20 (d, J = 7.3 Hz, 1H), 10.84 (s, 1H), 8.20-8.05 (m, 1H), 7.74-7.64 (m, |
| | | 1H), 7.60 (d, J = 8.0 Hz, 2H), 7.39 (d, J = 8.1 Hz, 2H), 7.12-6.97 (m, 4H), |
| | | 6.88 (s, 1H), 6.16 (s, 1H), 4.67-4.60 (m, 2H), 4.44-4.32 (m, 2H), 3.89-3.80 |
| | | (m, 1H), 3.68-3.59 (m, 2H), 3.57-3.47 (m, 2H), 3.21-3.11 (m, 6H), 3.10- |
| | | 2.99 (m, 6H), 2.88 (t, J = 11.4 Hz, 1H), 2.79-2.66 (m, 3H), 2.65-2.58 (m, |
| | | 2H), 2.36 (d, J = 7.0 Hz, 1H), 2.32-2.25 (m, 1H), 2.24-2.16 (m, 1H), 2.07- |
| | | 1.97 (m, 2H), 1.96 (s, 1H), 1.86-1.78 (m, 2H), 1.73-1.62 (m, 2H) |
| I-1167 | 894.4 | 12.17 (br d, J = 6.1 Hz, 1H), 10.92-10.73 (m, 1H), 8.19-8.02 (m, 1H), 7.74- |
| | | 7.64 (m, 1H), 7.63-7.55 (m, 2H), 7.38 (d, J = 8.3 Hz, 2H), 7.09-6.93 (m, |
| | | 4H), 6.86 (br s, 1H), 6.21-6.11 (m, 1H), 4.67-4.57 (m, 2H), 4.41-4.29 (m, |
| | | 2H), 3.82 (dd, J = 4.8, 11.8 Hz, 1H), 3.69-3.58 (m, 2H), 3.55-3.45 (m, 2H), |
| | | 3.28-3.23 (m, 2H), 3.18-2.99 (m, 11H), 2.88 (br t, J = 11.4 Hz, 1H), 2.77- |
| | | 2.64 (m, 3H), 2.62-2.57 (m, 1H), 2.35 (br d, J = 2.6 Hz, 1H), 2.27 (br d, J = |
| | | 7.3 Hz, 1H), 2.23-2.15 (m, 1H), 2.08-1.96 (m, 2H), 1.94-1.87 (m, 1H), 1.80 |
| | | (br d, J = 10.1 Hz, 2H), 1.73-1.60 (m, 2H) |
| I-1168 | 872.5 | 12.32-12.12 (m, 1H), 11.08 (s, 1H), 8.18-8.06 (m, 1H), 7.75-7.65 (m, 1H), |
| | | 7.56 (dt, J = 3.2, 8.0 Hz, 1H), 7.36-7.24 (m, 3H), 7.15-6.99 (m, 2H), 6.84 (d, |
| | | J = 6.8 Hz, 1H), 6.59 (s, 1H), 6.21-6.11 (m, 1H), 5.34-5.14 (m, 1H), 4.69- |
| | | 4.58 (m, 2H), 4.42-4.30 (m, 2H), 3.69-3.59 (m, 2H), 3.52 (d, J = 4.8 Hz, |
| | | 2H), 3.18-3.06 (m, 4H), 3.03-2.91 (m, 5H), 2.90-2.78 (m, 2H), 2.69-2.57 |
| | | (m, 2H), 2.39-2.26 (m, 2H), 2.01-1.85 (m, 5H), 1.47 (d, J = 2.8 Hz, 6H), |
| | | 1.15 (t, J = 7.2 Hz, 3H) |
| I-1169 | 858.5 | 12.21 (s, 1H), 11.07 (s, 1H), 8.16-8.05 (m, 1H), 7.73-7.65 (m, 1H), 7.54 (dt, |
| | | J = 2.8, 7.6 Hz, 1H), 7.39-7.22 (m, 3H), 7.14-6.98 (m, 2H), 6.83 (d, J = 6.4 |
| | | Hz, 1H), 6.62 (d, J = 2.4 Hz, 1H), 6.21-6.10 (m, 1H), 5.23 (d, J = 5.2 Hz, 1H), |
| | | 4.67-4.58 (m, 2H), 4.41-4.31 (m, 2H), 3.69-3.57 (m, 2H), 3.26-3.02 (m, |
| | | 8H), 2.99-2.90 (m, 4H), 2.89-2.77 (m, 2H), 2.69-2.56 (m, 2H), 2.38-2.25 |
| | | (m, 2H), 2.01-1.82 (m, 5H), 1.46 (d, J = 2.4 Hz, 6H) |
| I-1170 | 857.5 | 12.21 (d, J = 8.4 Hz, 1H), 11.08 (s, 1H), 7.78-7.67 (m, 1H), 7.60-7.50 (m, |
| | | 1H), 7.47-7.24 (m, 4H), 7.16-6.99 (m, 2H), 6.84 (d, J = 6. Hz, 1H), 6.64 (s, |
| | | 1H), 6.25-6.10 (m, 2H), 5.35-5.13 (m, 1H), 4.42-4.28 (m, 4H), 3.69-3.56 |
| | | (m, 2H), 3.25-3.03 (m, 6H), 3.01-2.92 (m, 6H), 2.90-2.78 (m, 2H), 2.69- |
| | | 2.57 (m, 2H), 2.37-2.24 (m, 2H), 2.04-1.82 (m, 5H), 1.47 (d, J = 2.4 Hz, 6H) |
| I-1171 | 757.3 | 10.92-10.81 (m, 2H), 8.12 (d, J = 3.2 Hz, 1H), 7.70 (s, 1H), 7.62-7.53 (m, |
| | | 3H), 7.46 (d, J = 8.0 Hz, 2H), 7.08-7.03 (m, 3H), 7.00-6.96 (m, 1H), 6.75- |
| | | 6.58 (m, 2H), 4.64 (t, J = 6.8 Hz, 2H), 4.38 (d, J = 12.0 Hz, 2H), 3.84-3.79 |
| | | (m, 1H), 3.64-3.56 (m, 2H), 3.50 (d, J = 11.6 Hz, 2H), 3.17-3.09 (m, 2H), |
| | | 3.00 (s, 6H), 2.83 (t, J = 10.4 Hz, 2H), 2.80-2.61 (m, 3H), 2.32 (d, J = 1.6 Hz, |
| | | 1H), 2.27-2.18 (m, 2H), 2.06-1.96 (m, 2H), 1.95 (s, 2H), 1.92-1.89 (m, 1H) |
| I-1172 | 835.5 | 13.82 (s, 1H), 11.07 (s, 1H), 8.20-8.02 (m, 2H), 7.75-7.62 (m, 1H), 7.35 (t, J = |
| | | 7.6 Hz, 1H), 7.19 (t, J = 8.0 Hz, 1H), 7.15-7.04 (m, 1H), 7.02-6.93 (m, |
| | | 2H), 6.85 (s, 1H), 6.22 (s, 1H), 5.30-5.11 (m, 1H), 4.66-4.59 (m, 2H), 4.41- |
| | | 4.32 (m, 2H), 3.63 (td, J = 5.6, 19.9 Hz, 2H), 3.18 (s, 4H), 3.13-3.06 (m, 2H), |
| | | 2.92-2.83 (m, 3H), 2.70 (s, 4H), 2.66-2.58 (m, 4H), 2.39-2.27 (m, 2H), |
| | | 1.96 (dd, J = 4.4, 9.6 Hz, 1H), 1.40 (d, J = 3.2 Hz, 6H) |
| I-1173 | 808.4 | 12.38-11.98 (m, 1H), 10.99-10.64 (m, 1H), 8.57 (s, 1H), 8.46 (s, 1H), 8.15- |
| | | 8.06 (m, 1H), 7.86 (d, J = 12.0 Hz, 1H), 7.74-7.62 (m, 1H), 7.39-7.28 (m, |
| | | 1H), 7.11-7.02 (m, 2H), 6.99 (d, J = 8.4 Hz, 1H), 6.93-6.87 (m, 1H), 6.19- |
| | | 6.10 (m, 1H), 4.67-4.58 (m, 2H), 4.41-4.31 (m, 2H), 3.86-3.78 (m, 1H), |
| | | 3.68-3.58 (m, 2H), 3.50 (d, J = 10.8 Hz, 4H), 3.13-3.06 (m, 4H), 2.89-2.78 |
| | | (m, 3H), 2.73-2.60 (m, 2H), 2.36 (d, J = 1.6 Hz, 1H), 2.30-2.26 (m, 1H), |
| | | 2.24-2.18 (m, 1H), 2.08-1.88 (m, 6H), 1.16 (t, J = 7.2 Hz, 3H) |
| I-1174 | 924.5 | 12.25-12.16 (m, 1H), 10.45 (s, 1H), 7.79-7.68 (m, 1H), 7.45 (br d, J = 10.4 |
| | | Hz, 1H), 7.33-7.23 (m, 2H), 7.19 (br d, J = 8.0 Hz, 1H), 7.05-6.91 (m, 2H), |
| | | 6.64 (br s, 1H), 6.26-6.07 (m, 2H), 4.39-4.31 (m, 3H), 3.71 (br d, J = 5.6 Hz, |
| | | 3H), 3.58 (br s, 3H), 3.51 (br d, J = 10.0 Hz, 3H), 3.17-3.03 (m, 6H), 3.01- |
| | | 2.95 (m, 3H), 2.90 (br d, J = 9.2 Hz, 2H), 2.81-2.70 (m, 5H), 2.33 (br s, 1H), |
| | | 2.27 (br d, J = 1.2 Hz, 1H), 2.14 (br t, J = 10.8 Hz, 2H), 1.84 (br d, J = 10.8 Hz, |
| | | 2H), 1.69-1.56 (m, 4H), 1.54-1.44 (m, 1H), 1.25-1.13 (m, 2H) |
| I-1175 | 876.5 | 12.46-12.09 (m, 1H), 11.20-10.92 (m, 1H), 8.16-8.08 (m, 1H), 7.73-7.65 |
| | | (m, 1H), 7.45-7.33 (m, 2H), 7.32-7.25 (m, 1H), 7.17-7.04 (m, 2H), 6.84 (d, |
| | | J = 6.8 Hz, 1H), 6.73-6.65 (m, 1H), 6.22-6.12 (m, 1H), 5.32-5.17 (m, 1H), |
| | | 4.67-4.60 (m, 2H), 4.41-4.33 (m, 2H), 3.68-3.59 (m, 2H), 3.21-3.06 (m, |
| | | 8H), 2.99 (s, 5H), 2.87 (d, J = 13.6 Hz, 1H), 2.70-2.58 (m, 2H), 2.38-2.27 |
| | | (m, 2H), 2.00-1.91 (m, 5H), 1.47 (d, J = 2.8 Hz, 6H) |
| I-1176 | 808.4 | 12.42-11.95 (m, 1H), 11.03-10.52 (m, 1H), 8.73-8.46 (m, 1H), 8.18-8.01 |
| | | (m, 1H), 7.92-7.82 (m, 1H), 7.74-7.63 (m, 1H), 7.42-7.27 (m, 1H), 6.99- |
| | | 6.83 (m, 3H), 6.14 (br s, 1H), 4.67-4.58 (m, 2H), 4.42-4.31 (m, 2H), 4.07- |
| | | 3.96 (m, 1H), 3.69-3.58 (m, 2H), 3.52-3.44 (m, 2H), 3.20-3.04 (m, 8H), |
| | | 2.89-2.70 (m, 4H), 2.38-2.27 (m, 3H), 2.21-2.12 (m, 4H), 2.03-1.90 (m, |
| | | 5H) |
| I-1177 | 876.6 | 11.15-10.94 (m, 1H), 8.14-8.08 (m, 1H), 7.72-7.67 (m, 1H), 7.64-7.58 (m, |
| | | 1H), 7.49-7.43 (m, 4H), 7.32-7.24 (m, 1H), 7.12 (d, J = 8.0 Hz, 1H), 6.99 (d, |
| | | J = 1.2 Hz, 1H), 6.83 (d, J = 6.8 Hz, 1H), 6.79-6.62 (m, 1H), 6.12-6.00 (m, |
| | | 1H), 5.32-5.17 (m, 1H), 4.63 (t, J = 6.8 Hz, 2H), 4.24 (s, 2H), 3.71-3.55 (m, |
| | | 4H), 3.15-3.08 (m, 2H), 3.03-2.93 (m, 10H), 2.90-2.78 (m, 2H), 2.69-2.57 |
| | | (m, 2H), 2.34-2.24 (m, 2H), 2.00-1.85 (m, 5H), 1.46 (d, J = 2.8 Hz, 6H), |
| | | 0.56-0.38 (m, 1H), 0.08-−0.02 (m, 2H), −0.28-−0.36 (m, 2H) |
| I-1178 | 770.5 | 14.13-13.48 (m, 1H), 11.32-10.74 (m, 1H), 8.26 (d, J = 2.4 Hz, 1H), 8.12 (d, |
| | | J = 12.4 Hz, 1H), 7.77-7.65 (m, 3H), 7.49 (d, J = 8.4 Hz, 2H), 7.33-7.22 (m, |
| | | 1H), 7.20-7.08 (m, 2H), 6.89-6.77 (m, 1H), 6.25 (s, 1H), 5.33-5.16 (m, |
| | | 1H), 4.67-4.60 (m, 2H), 4.39 (d, J = 17.6 Hz, 2H), 3.70-3.60 (m, 2H), 3.15- |
| | | 3.07 (m, 2H), 2.98 (s, 4H), 2.91-2.77 (m, 2H), 2.69-2.60 (m, 2H), 2.40- |
| | | 2.28 (m, 2H), 2.00-1.86 (m, 5H), 1.47 (d, J = 2.8 Hz, 6H) |
| I-1179 | 825.5 | 12.25 (br d, J = 0.8 Hz, 1H), 10.82 (s, 1H), 8.47 (s, 1H), 8.16-8.06 (m, 1H), |
| | | 7.74-7.64 (m, 1H), 7.49-7.39 (m, 2H), 7.13-7.03 (m, 3H), 7.02-6.96 (m, |
| | | 1H), 6.57-6.57 (m, 1H), 6.62 (br s, 1H), 6.18 (br d, J = 9.2 Hz, 1H), 4.63 (q, J = |
| | | 7.2 Hz, 2H), 4.42-4.31 (m, 2H), 3.83 (br dd, J = 4.8, 11.6 Hz, 1H), 3.66 (br |
| | | t, J = 5.6 Hz, 1H), 3.62-3.59 (m, 1H), 3.50 (br d, J = 10.8 Hz, 4H), 3.15-3.07 |
| | | (m, 4H), 3.03 (dt, J = 4.4, 6.8 Hz, 2H), 2.84 (br t, J = 10.2 Hz, 2H), 2.69-2.62 |
| | | (m, 2H), 2.37-2.34 (m, 1H), 2.28 (br s, 1H), 2.24-2.17 (m, 1H), 2.06-1.97 |
| | | (m, 2H), 1.93 (br s, 3H), 1.15 (br t, J = 6.8 Hz, 3H) |
| I-1180 | 755.5 | 14.05-13.39 (m, 1H), 11.36-10.67 (m, 1H), 8.11 (d, J = 10.4 Hz, 1H), 7.81 |
| | | (br d, J = 2.0 Hz, 1H), 7.69 (d, J = 11.2 Hz, 1H), 7.35 (s, 1H), 7.32-7.24 (m, |
| | | 3H), 7.22-7.16 (m, 1H), 7.01-6.88 (m, 1H), 6.69 (d, J = 7.2 Hz, 1H), 6.23 (br |
| | | d, J = 9.6 Hz, 1H), 5.70 (dd, J = 5.2, 11.8 Hz, 1H), 4.63 (q, J = 7.2 Hz, 2H), |
| | | 4.38 (br d, J = 18.0 Hz, 2H), 3.69-3.59 (m, 2H), 3.48 (br d, J = 8.8 Hz, 2H), |
| | | 3.14-3.08 (m, 2H), 2.91-2.82 (m, 3H), 2.78-2.71 (m, 2H), 2.71-2.65 (m, |
| | | 4H), 2.40-2.28 (m, 2H), 2.24-2.18 (m, 4H), 2.05-1.96 (m, 4H) |
| I-1181 | 755.5 | 13.84-13.66 (m, 1H), 10.88 (s, 1H), 8.10 (d, J = 10.8 Hz, 1H), 7.85-7.74 (m, |
| | | 1H), 7.72-7.61 (m, 1H), 7.43-7.34 (m, 2H), 7.30-7.22 (m, 2H), 7.10-7.01 |
| | | (m, 2H), 7.01-6.88 (m, 1H), 6.27-6.15 (m, 1H), 4.67-4.56 (m, 2H), 4.40- |
| | | 4.38 (m, 1H), 4.36-4.34 (m, 1H), 4.32 (s, 2H), 3.68-3.58 (m, 2H), 3.41- |
| | | 3.36 (m, 2H), 3.13-3.07 (m, 2H), 2.95-2.84 (m, 2H), 2.81-2.73 (m, 1H), |
| | | 2.69-2.61 (m, 2H), 2.54 (s, 2H), 2.39-2.32 (m, 2H), 2.31-2.26 (m, 1H), |
| | | 2.23-2.16 (m, 4H), 2.06-1.97 (m, 4H) |
| I-1297 | 887.2 | 12.90-12.66 (m, 1H), 11.09 (s, 1H), 8.12 (t, J = 10.0 Hz, 1H), 7.78-7.58 (m, |
| | | 1H), 7.44-6.88 (m, 5H), 6.73-6.50 (m, 2H), 6.25-5.97 (m, 1H), 5.38 (dd, J = |
| | | 5.6, 12.8 Hz, 1H), 4.69-4.55 (m, 2H), 4.41-4.24 (m, 2H), 3.77-3.69 (m, |
| | | 3H), 3.67-3.55 (m, 5H), 3.30-3.25 (m, 6H), 3.17-3.07 (m, 4H), 3.05 (s, |
| | | 3H), 2.95-2.84 (m, 1H), 2.78-2.60 (m, 9H), 2.38-2.25 (m, 2H), 2.06-1.95 |
| | | (m, 1H) |
| I-1308 | 888.3 | 11.10 (s, 1H), 10.38 (s, 1H), 8.46 (s, 1H), 8.13 (s, 1H), 7.76-7.66 (m, 2H), |
| | | 7.60 (d, J = 12.0 Hz, 1H), 7.27-7.16 (m, 2H), 7.11 (s, 1H), 7.03-6.92 (m, |
| | | 3H), 6.75-6.48 (m, 4H), 5.38 (dd, J = 3.2, 12.4 Hz, 1H), 4.68-4.61 (m, 2H), |
| | | 4.39 (d, J = 10.4 Hz, 2H), 3.75 (s, 3H), 3.65-3.55 (m, 5H), 3.29 (s, 2H), 3.18- |
| | | 3.07 (m, 4H), 2.97-2.85 (m, 1H), 2.71-2.60 (m, 8H), 2.44-2.36 (m, 4H), |
| | | 2.33-2.28 (m, 1H), 2.24-2.18 (m, 1H), 2.05-1.82 (m, 2H) |
| I-1412 | 999.2 | 12.20 (s, 1H), 11.13-11.03 (m, 1H), 10.43 (br s, 1H), 9.26 (s, 1H), 8.14-8.10 |
| | | (m, 1H), 8.00 (d, J = 8.0 Hz, 1H), 7.82 (d, J = 7.6 Hz, 1H), 7.71-7.61 (m, 3H), |
| | | 7.40 (br s, 1H), 7.23 (d, J = 8.4 Hz, 1H), 7.00-6.88 (m, 4H), 6.73 (s, 1H), 6.65 |
| | | (br d, J = 8.0 Hz, 1H), 6.19-6.10 (m, 1H), 5.43-5.32 (m, 1H), 4.67-4.58 (m, |
| | | 2H), 4.42-4.30 (m, 2H), 3.80-3.75 (m, 3H), 3.67-3.64 (m, 2H), 3.61 (s, |
| | | 3H), 3.27 (br s, 3H), 3.15-3.07 (m, 4H), 2.94-2.83 (m, 1H), 2.72-2.63 (m, |
| | | 8H), 2.61 (s, 3H), 2.39-2.26 (m, 3H), 2.04-1.96 (m, 1H) |
| I-1307 | 877.2 | 11.10 (s, 1H), 10.44 (s, 1H), 8.13 (s, 1H), 7.76-7.66 (m, 2H), 7.27-7.16 (m, |
| | | 3H), 7.03-6.92 (m, 4H), 6.75-6.48 (m, 4H), 5.38 (dd, J = 3.2, 12.4 Hz, 1H), |
| | | 4.68-4.61 (m, 2H), 4.39 (d, J = 10.4 Hz, 2H), 3.75-3.70 (m, 6H), 3.63 (s, |
| | | 3H), 3.63-3.60 (m, 1H), 3.29-3.23 (m, 4H), 3.18-3.07 (m, 4H), 2.97-2.85 |
| | | (m, 1H), 2.71-2.60 (m, 8H), 2.44-2.36 (m, 3H), 2.05-1.82 (m, 2H) |
| I-1278 | 869.3 | 11.99-11.84 (m, 1H), 11.09 (s, 1H), 8.13 (s, 1H), 7.70 (s, 1H), 7.40-7.34 (m, |
| | | 1H), 7.30 (d, J = 8.4 Hz, 1H), 6.99 (d, J = 5.2 Hz, 2H), 6.96-6.91 (m, 1H), |
| | | 6.83-6.69 (m, 2H), 6.65 (d, J = 8.4 Hz, 1H), 6.54-6.50 (d, J = 1.6 Hz, 1H), |
| | | 5.38 (dd, J = 5.2, 12.4 Hz, 1H), 4.69-4.61 (m, 2H), 4.59-4.48 (m, 2H), 3.77 |
| | | (s, 3H), 3.68-3.64 (m, 1H), 3.64-3.60 (m, 3H), 3.59-3.55 (m, 1H), 3.30- |
| | | 3.28 (m, 3H), 3.18-3.07 (m, 8H), 2.98-2.84 (m, 2H), 2.76-2.57 (m, 10H), |
| | | 2.39-2.35 (m, 1H), 2.34-2.28 (m, 1H), 2.06-1.96 (m, 1H) |
| I-1298 | 887.5 | 11.09 (s, 1H), 8.10 (d, J = 10.0 Hz, 1H), 7.68 (d, J = 10.0 Hz, 1H), 7.27 (t, J = |
| | | 7.6 Hz, 1H), 7.02-6.81 (m, 5H), 6.69-6.59 (m, 2H), 6.36 (s, 1H), 5.38 (dd, J = |
| | | 5.6, 12.6 Hz, 1H), 4.62 (q, J = 7.2 Hz, 2H), 4.38 (d, J = 17.6 Hz, 2H), 3.70- |
| | | 3.56 (m, 8H), 3.31-3.28 (m, 3H), 3.18-3.06 (m, 7H), 2.98 (s, 3H), 2.95- |
| | | 2.84 (m, 1H), 2.78-2.58 (m, 9H), 2.41-2.24 (m, 2H), 2.05-1.96 (m, 1H) |
| I-1296 | 887.2 | 11.55-11.37 (m, 1H), 11.19-10.98 (m, 1H), 8.25-8.02 (m, 1H), 7.78-7.62 |
| | | (m, 1H), 7.44-7.26 (m, 1H), 7.01-6.93 (m, 3H), 6.82 (s, 1H), 6.73-6.61 (m, |
| | | 2H), 6.60-6.50 (m, 1H), 5.38 (dd, J = 4.8, 12.4 Hz, 1H), 4.68-4.58 (m, 2H), |
| | | 4.54-4.45 (m, 2H), 3.77-3.72 (m, 3H), 3.70-3.52 (m, 5H), 3.18-3.10 (m, |
| | | 6H), 3.08-3.05 (m, 2H), 3.02-2.98 (m, 3H), 2.94-2.75 (m, 2H), 2.74-2.64 |
| | | (m, 8H), 2.60-2.55 (m, 2H), 2.39-2.31 (m, 2H), 2.06-1.97 (m, 1H) |
| I-1448 | 811.1 | 10.82 (s, 1H), 8.26 (d, J = 1.6 Hz, 1H), 8.14-8.06 (m, 1H), 7.73-7.61 (m, |
| | | 1H), 7.61-7.53 (m, 1H), 7.52-7.40 (m, 1H), 7.35-7.21 (m, 1H), 7.11 (t, J = |
| | | 8.8 Hz, 1H), 7.01-6.91 (m, 1H), 6.87-6.73 (m, 2H), 4.61 (t, J = 6.8 Hz, 2H), |
| | | 4.50-4.37 (m, 2H), 3.90 (dd, J = 4.4, 12.0 Hz, 3H), 3.67-3.56 (m, 2H), 3.19- |
| | | 3.05 (m, 3H), 3.05-2.98 (m, 6H), 2.87 (t, J = 10.8 Hz, 2H), 2.77-2.67 (m, |
| | | 1H), 2.53 (s, 1H), 2.45-2.32 (m, 2H), 2.16 (dq, J = 4.0, 12.8 Hz, 1H), 2.01- |
| | | 1.82 (m, 5H) |
| I-1449 | 847.5 | 10.89 (s, 1H), 8.26 (d, J = 1.6 Hz, 1H), 8.14-8.06 (m, 1H), 7.71-7.65 (m, |
| | | 1H), 7.62-7.55 (m, 1H), 7.54-7.36 (m, 3H), 7.14-7.09 (m, 1H), 7.08-7.02 |
| | | (m, 1H), 7.01-6.93 (m, 1H), 4.61 (t, J = 6.8 Hz, 2H), 4.49-4.41 (m, 2H), |
| | | 4.38-4.35 (m, 1H), 4.33 (s, 3H), 3.65 (t, J = 5.6 Hz, 1H), 3.60 (t, J = 5.2 Hz, |
| | | 1H), 3.43-3.36 (m, 2H), 3.20-3.07 (m, 3H), 3.02 (s, 6H), 2.94 (s, 2H), 2.69- |
| | | 2.60 (m, 2H), 2.44 (s, 1H), 2.40-2.29 (m, 2H), 2.23-2.16 (m, 1H), 2.14- |
| | | 2.06 (m, 2H), 2.01 (d, J = 14.0 Hz, 2H) |
| I-1450 | 939.4 | 12.20 (s, 1H), 11.07 (s, 1H), 7.78-7.65 (m, 1H), 7.47-7.34 (m, 1H), 7.28- |
| | | 7.13 (m, 4H), 7.03-6.87 (m, 2H), 6.82-6.76 (m, 1H), 6.74-6.68 (m, 1H) 6.65- |
| | | 6.60 (m, 1H), 6.25-6.12 (m, 2H), 4.41-3.35 (m, 4H), 3.81-3.74 (m, |
| | | 2H), 3.68-3.60 (m, 4H), 3.24-3.11 (m, 8H), 2.99-2.95 (m, 3H), 2.79-2.73 |
| | | (m, 3H), 2.69-2.61 (m, 3H), 2.41-2.30 (m, 6H), 1.89-1.81 (m, 2H), 1.68- |
| | | 1.62 (m, 2H), 1.60-1.53 (m, 4H) |
| I-1335 | 868.6 | 11.10 (s, 1H), 8.12 (d, J = 5.6 Hz, 1H), 7.74-7.60 (m, 3H), 7.17 (dd, J = 4.8, |
| | | 8.0 Hz, 1H), 7.03-6.75 (m, 5H), 6.70 (s, 1H), 6.64 (d, J = 8.8 Hz, 1H), 6.11 |
| | | (d, J = 18.0 Hz, 1H), 5.38 (dd, J = 5.2, 12.4 Hz, 1H), 4.68-4.59 (m, 2H), 4.42- |
| | | 4.29 (m, 2H), 3.73 (s, 3H), 3.67-3.62 (m, 3H), 3.62-3.57 (m, 2H), 3.31- |
| | | 3.26 (m, 3H), 3.18-3.08 (m, 4H), 2.99 (s, 6H), 2.92-2.83 (m, 1H), 2.78- |
| | | 2.71 (m, 1H), 2.70-2.63 (m, 7H), 2.62-2.58 (m, 1H), 2.38-2.32 (m, 1H), |
| | | 2.30-2.25 (m, 1H), 2.05-1.94 (m, 1H) |
| I-1453 | 745.5 | 12.38-11.87 (m, 1H), 10.95-10.63 (m, 1H), 7.63 (d, J = 8.0 Hz, 2H), 7.44 (d, |
| | | J = 8.0 Hz, 2H), 7.10-7.01 (m, 3H), 6.98 (d, J = 8.4 Hz, 1H), 6.88 (d, J = 3.2 |
| | | Hz, 1H), 6.21 (s, 1H), 4.74-4.26 (m, 2H), 4.04-3.60 (m, 3H), 3.49 (d, J = |
| | | 11.2 Hz, 2H), 3.25-2.93 (m, 6H), 2.90-2.58 (m, 5H), 2.54-2.51 (m, 2H), |
| | | 2.28-2.12 (m, 1H), 2.02 (td, J = 4.4, 8.8 Hz, 1H), 1.98-1.82 (m, 4H), 1.68- |
| | | 1.59 (m, 2H), 1.54 (s, 2H) |
| I-1397 | 881.5 | 11.89-12.02 (m, 1 H) 10.87 (s, 1 H) 8.08-8.21 (m, 2 H) 7.98 (d, J = 8.38 Hz, 1 |
| | | H) 7.64-7.73 (m, 1 H) 7.50-7.57 (m, 1 H) 7.41-7.49 (m, 1 H) 7.19 (dd, |
| | | J = 13.45, 8.19 Hz, 2 H) 6.78-6.92 (m, 1 H) 6.57-6.70 (m, 3 H) 6.44-6.53 |
| | | (m, 1 H) 6.04-6.16 (m, 2 H) 4.52-4.68 (m, 3 H) 4.26-4.40 (m, 2 H) 3.69- |
| | | 3.75 (m, 3 H) 3.57-3.67 (m, 2 H) 3.28 (br d, J = 2.63 Hz, 6 H) 3.11 (td, |
| | | J = 14.29, 7.32 Hz, 8 H) 2.79-2.88 (m, 1 H) 2.59-2.72 (m, 7 H) 2.24-2.38 |
| | | (m, 2 H) 2.05-2.22 (m, 2 H) |
| I-1279 | 886.2 | 11.10 (s, 1H), 8.12 (s, 1H), 7.72-7.65 (m, 1H), 7.63-7.54 (m, 1H), 7.31 (d, J = |
| | | 2.8 Hz, 1H), 7.26 (d, J = 8.4 Hz, 1H), 7.04-6.88 (m, 4H), 6.75-6.62 (m, |
| | | 2H), 5.37 (dd, J = 5.6, 12.8 Hz, 1H), 4.69-4.60 (m, 2H), 4.58-4.47 (m, 2H), |
| | | 3.75 (s, 3H), 3.68-3.63 (m, 1H), 3.62 (s, 3H), 3.60-3.56 (m, 1H), 3.55-3.55 |
| | | (m, 1H), 3.26-2.98 (m, 11H), 2.98-2.80 (m, 2H), 2.77-2.59 (m, 9H), 2.41- |
| | | 2.38 (m, 1H), 2.35-2.31 (m, 1H), 2.08-1.94 (m, 1H) |
| I-1288 | 924.7 | 11.94 (d, J = 4.8 Hz, 1H), 11.10 (s, 1H), 8.13-8.06 (m, 1H), 7.72-7.61 (m, |
| | | 1H), 7.22-7.13 (m, 1H), 6.99 (d, J = 5.6 Hz, 2H), 6.97-6.93 (m, 2H), 6.67 (s, |
| | | 1H), 6.63-6.57 (m, 1H), 6.47 (d, J = 2.8 Hz, 1H), 5.43-5.34 (m, 1H), 4.80- |
| | | 4.67 (m, 1H), 4.66-4.57 (m, 2H), 4.47 (br d, J = 12.3 Hz, 1H), 4.35-4.19 (m, |
| | | 1H), 3.98 (d, J = 9.2 Hz, 1H), 3.71 (d, J = 1.2 Hz, 3H), 3.62 (s, 3H), 3.26 (s, |
| | | 6H), 3.13 (d, J = 6.8 Hz, 7H), 3.04-2.82 (m, 4H), 2.71-2.61 (m, 8H), 2.39- |
| | | 2.28 (m, 1H), 2.06-1.95 (m, 1H) |
| I-1273 | 895.3 | 11.90-11.81 (m, 1H), 11.09 (br s, 1H), 8.15-8.08 (m, 1H), 7.72-7.65 (m, |
| | | 1H), 7.31 (s, 1H), 7.21 (br d, J = 8.3 Hz, 1H), 7.04 (s, 1H), 7.01-6.92 (m, 3H), |
| | | 6.89-6.80 (m, 1H), 6.69 (br s, 1H), 6.61 (br d, J = 9.3 Hz, 1H), 6.50 (br s, |
| | | 1H), 6.14-6.06 (m, 1H), 5.41-5.34 (m, 1H), 4.68-4.59 (m, 2H), 4.40-4.28 |
| | | (m, 2H), 3.81 (s, 3H), 3.74 (br d, J = 3.6 Hz, 3H), 3.67-3.59 (m, 5H), 3.28 (br |
| | | d, J = 3.0 Hz, 4H), 3.17-3.08 (m, 4H), 2.94-2.85 (m, 1H), 2.74-2.65 (m, |
| | | 8H), 2.36-2.27 (m, 2H), 2.05-1.96 (m, 1H) |
| I-1533 | 918.5 | 11.91-11.80 (m, 1H), 11.10 (s, 1H), 8.16-8.03 (m, 1H), 7.77-7.62 (m, 1H), |
| | | 7.16 (d, J = 8.4 Hz, 1H), 7.02-6.92 (m, 3H), 6.88 (d, J = 5.6 Hz, 1H), 6.67 (s, |
| | | 1H), 6.60 (d, J = 8.0 Hz, 1H), 6.46 (s, 1H), 5.38 (dd, J = 4.8, 12.8 Hz, 1H), 4.94- |
| | | 4.70 (m, 1H), 4.63-4.58 (m, 2H), 4.54-4.39 (m, 1H), 4.08-3.92 (m, 1H), |
| | | 3.87-3.75 (m, 1H), 3.71 (s, 3H), 3.62 (s, 3H), 3.55-3.45 (m, 1H), 3.26 (s, |
| | | 6H), 3.20-3.00 (m, 9H), 2.94-2.81 (m, 2H), 2.74-2.62 (m, 8H), 2.08-1.93 |
| | | (m, 2H), 1.85-1.81 (m, 1H), 1.79-1.63 (m, 2H). |

^{a}The cross coupling was performed under standard techniques from 30-80° C. for 1-3 hrs. Other catalysts systems could be utilized, such as Pd(dppf)Cl₂, and bases, such as K₃PO₄. Other halides such as bromides could also be utilized. Final compounds were purified via standard techniques including prep-HPLC and other chromatography techniques.
^{b}LC-MS (ESI+) m/z reported as (M/2 + H)⁺.

### Example 3 (Method 3): Synthesis of 5-(1-(3-(1H-pyrazol-1-yl)propanoyl)-1,2,5,6-tetrahydropyridin-3-yl)-7-(4-(1-(4-chloro-3-(2,6-dioxopiperidin-3-yl)benzoyl)azetidin-3-yl)phenyl)-4-fluoro-N,N-dimethylbenzofuran-2-carboxamide (I-495)

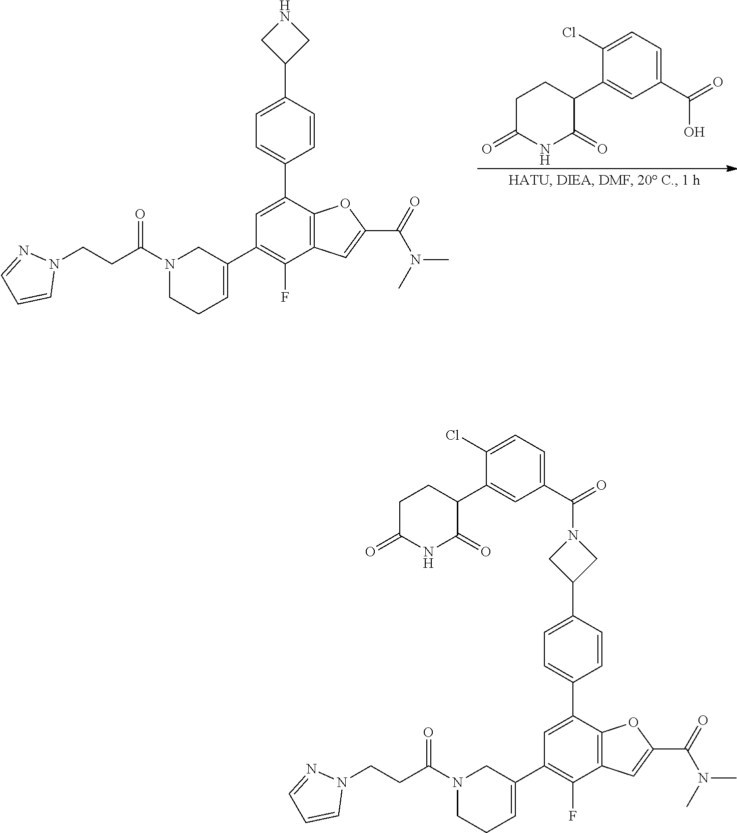 [SMILES: CN(C)C(=O)c1cc2c(F)c(C3=CCCN(C(=O)CCn4cccn4)C3)cc(-c3ccc(C4CNC4)cc3)c2o1 ; O=C1CCC(c2cc(C(=O)O)ccc2Cl)C(=O)N1 ; CN(C)C(=O)c1cc2c(F)c(C3=CCCN(C(=O)CCn4cccn4)C3)cc(-c3ccc(C4CN(C(=O)c5ccc(Cl)c(C6CCC(=O)NC6=O)c5)C4)cc3)c2o1]

To a solution of 5-(1-(3-(1H-pyrazol-1-yl)propanoyl)-1,2,5,6-tetrahydropyridin-3-yl)-7-(4-(azetidin-3-yl)phenyl)-4-fluoro-N,N-dimethylbenzofuran-2-carboxamide (128 mg, 135 μmol, 6HCl) and 4-chloro-3-(2,6-dioxopiperidin-3-yl)benzoic acid (36.0 mg, 135 μmol, form) in DMF (1.00 mL) was added HATU (102 mg, 269 μmol) and DIEA (174 mg, 1.35 mmol). Then the mixture was stirred at 20° C. for 2 hr. On completion, the reaction mixture was filtered to give a filtrate. The residue was purified by prep-HPLC (column: Phenomenex luna C18 150*25 mm*10 um; mobile phase: [water (FA)-ACN]; gradient: 40%-60% B over 10 min) to afford the title compound (37.1 mg, 33% yield, FA) as an off-white solid. LC-MS (ESI⁺) m/z 791.3 (M+H)⁺; ¹H NMR (400 MHz, DMSO-d₆) δ ppm 10.94 (s, 1H), 7.87 (d, J=8.0 Hz, 2H), 7.71 (J=2.0, 11.2 Hz, 1H), 7.69-7.62 (m, 2H), 7.62-7.51 (m, 5H), 7.45-7.35 (m, 1H), 6.25-6.11 (m, 2H), 4.77-4.64 (m, 1H), 4.52 (t, J=9.2 Hz, 1H), 4.48-4.40 (m, 1H), 4.40-4.27 (m, 5H), 4.17-4.08 (m, 1H), 4.07-3.96 (m, 1H), 3.69-3.62 (m, 1H), 3.59 (t, J=5.6 Hz, 1H), 3.27 (s, 3H), 3.08-3.00 (m, 3H), 3.00-2.93 (m, 2H), 2.84-2.72 (m, 1H), 2.57 (s, 1H), 2.41-2.24 (m, 3H), 2.06-1.95 (m, 1H).

TABLE 7
Compounds synthesized via Method 3, the coupling
of the corresponding amines and carboxylic acids

| | | | LCMS | |
|---|---|---|---|---|
| | | | (ESI+) | |
| | | | m/z | |
| I-# | Amine | Acid | (M + H)⁺ | ¹H NMR (400 MHZ, DMSO-d6) δ |
| I-1048 | OO | OQ | 801.3 | 12.2-12.0 (m, 1H), 10.9-10.8 (m, 1H), 8.11 (d, J = 12.0 |
| | | | | Hz, 1H), 7.69 (d, J = 11.6 Hz, 1H), 7.62 (br d, J = 7.6 Hz, |
| | | | | 2H), 7.47-7.41 (m, 2H), 7.10-6.96 (m, 5H), 6.17 (br d, |
| | | | | J = 4.8 Hz, 1H), 4.67-4.59 (m, 2H), 4.41-4.32 (m, 2H), |
| | | | | 3.82 (dd, J = 4.8 12.0 Hz, 1H), 3.68-3.58 (m, 2H), 3.49 |
| | | | | (br d, J = 11.6 Hz, 2H), 3.14-3.07 (m, 3H), 3.05 (s, 3H), |
| | | | | 2.81 (br t, J = 11.2 Hz, 2H), 2.75-2.69 (m, 1H), |
| | | | | 2.68-2.61 (m, 1H), 2.40-2.31 (m, 2H), 2.30-2.25 (m, 1H), |
| | | | | 2.24-2.18 (m, IH), 2.02 (dt, J = 4.4, 8.8 Hz, 1H), 1.96- |
| | | | | 1.85 (m, 4H), 0.79-0.71 (m, 2H), 0.61 (br s, 2H) |

TABLE 8
Compounds synthesized via Method 3, coupling of amines and carboxylic acids

| | LCMS | |
|---|---|---|
| | (ESI+) | |
| | m/z | |
| I-# | (M + H)⁺ | ¹H NMR (400 MHz, DMSO-d₆) δ |
| I-79 | 878.3 | 11.57-11.48 (m, 1H), 11.15-11.07 (m, 1H), 8.45 (d, J = 2.4 Hz, 1H), 8.15-8.09 |
| | | (m, 1H), 7.88-7.80 (m, 1H), 7.70 (d, J = 2.0 Hz, 1H), 7.56 (dd, J = 1.6, 8.8 Hz, 1H), |
| | | 7.48-7.35 (m, 2H), 7.31-7.24 (m, 1H), 7.23-7.11 (m, 2H), 6.93-6.87 (m, 1H), |
| | | 6.72 (d, J = 2.8 Hz, 1H), 6.31-6.22 (m, 1H), 5.39 (dd, J = 4.8, 12.8 Hz, 1H), 4.68- |
| | | 4.58 (m, 2H), 4.51-4.25 (m, 6H), 3.63-3.53 (m, 2H), 3.39 (s, 3H), 3.18-3.06 (m, |
| | | 3H), 2.88-2.61 (m, 6H), 2.29-2.22 (m, 1H), 2.07-1.99 (m, 1H), 1.85-1.67 (m, |
| | | 4H), 1.60-1.43 (m, 2H), 1.37-1.21 (m, 5H), 1.20-1.04 (m, 4H) |
| I-76 | 840.4 | 11.42 (d, J = 8.4 Hz, 1H), 11.08 (s, 1H), 8.13 (d, J = 2.4 Hz, 1H), 7.70 (s, 1H), 7.62 |
| | | (s, 4H), 7.05-6.73 (m, 4H), 5.36 (dd, J = 5.6, 12.4 Hz, 1H), 4.66 (t, J = 6.4 Hz, 2H), |
| | | 3.86-3.58 (m, 8H), 3.55 (s, 3H), 3.52 (d, J = 3.2 Hz, 4H), 3.47 (s, 3H), 3.46 (s, 4H), |
| | | 3.31-3.24 (m, 2H), 3.22-3.04 (m, 4H), 3.04-2.55 (m, 4H), 2.49-2.33 (m, 3H), |
| | | 2.09-1.77 (m, 3H). |
| I-75 | 752.2 | 11.43 (d, J = 9.2 Hz, 1H), 11.08 (s, 1H), 8.20-8.09 (m, 1H), 7.70 (s, 1H), 7.64-7.25 |
| | | (m, 3H), 7.24-7.10 (m, 1H), 7.03-6.75 (m, 4H), 5.35 (dd, J = 5.2, 12.4 Hz, 1H), |
| | | 4.66 (t, J = 6.4 Hz, 2H), 3.81-3.60 (m, 8H), 3.54 (s, 6H), 3.31-3.25 (m, 2H), |
| | | 3.20-3.09 (m, 3H), 2.94-2.82 (m, 1H), 2.75-2.56 (m, 3H), 2.51 (s, |
| | | 2H), 2.47 (s, 2H), 2.06-1.80 (m, 3H) |
| I-67 | 841.4 | 11.58-11.43 (m, 1H), 11.09 (s, 1H), 8.22 (s, 1H), 8.17-8.13 (m, 1H), 8.12 (s, 1H), |
| | | 7.87 (s, 1H), 7.72-7.67 (m, 1H), 7.58 (d, J = 8.4 Hz, 1H), 7.46-7.36 (m, 2H), 7.26 |
| | | (dd, J = 1.6, 8.4 Hz, 1H), 7.23-7.15 (m, 1H), 7.08 (d, J = 8.0 Hz, 1H), 6.92-6.78 |
| | | (m, 1H), 6.35-6.16 (m, 1H), 5.35 (dd, J = 5.6, 12.6 Hz, 1H), 4.72-4.57 (m, 2H), |
| | | 4.47-4.30 (m, 2H), 4.25-4.07 (m, 1H), 3.65-3.54 (m, 4H), 3.36 (s, 3H), 3.16-2.98 |
| | | (m, 6H), 2.94-2.86 (m, 1H), 2.81-2.69 (m, 1H), 2.67-2.60 (m, 1H), 2.48-2.40 |
| | | (m, 2H), 2.35-1.86 (m, 10H), 1.74-1.57 (m, 2H), 1.57-1.39 (m, 2H) |
| I-66 | 840.2 | 11.52-11.29 (m, 1H), 8.14 (s, 1H), 7.70 (s, 1H), 7.63-7.53 (m, 1H), 7.39 (s, 1H), |
| | | 7.29-7.13 (m, 2H), 7.07-6.98 (m, 2H), 6.92-6.79 (m, 3H), 5.32 (dd, J = 4.8, 13.2 |
| | | Hz, 1H), 4.85-4.72 (m, 1H), 4.66 (t, J = 6.4 Hz, 2H), 3.83-3.45 (m, 25H), 3.22- |
| | | 3.13 (m, 2H), 2.85-2.60 (m, 7H), 2.04-1.85 (m, 3H) |
| I-52 | 776.4 | 11.76-11.56 (m, 1H), 10.79 (s, 1H), 8.12 (s, 1H), 7.69 (d, J = 3.2 Hz, 1H), 7.42- |
| | | 7.36 (m, 1H), 7.26-7.17 (m, 1H), 6.93-6.72 (m, 2H), 6.51-6.33 (m, 2H), 6.27 (s, |
| | | 1H), 4.71-4.50 (m, 3H), 4.42-4.21 (m, 3H), 4.04 (d, J = 13.2 Hz, 1H), 3.64-3.52 |
| | | (m, 2H), 3.20-3.03 (m, 6H), 2.88 (s, 2H), 2.81-2.63 (m, 6H), 2.63-2.51 (m, 4H), |
| | | 2.32-2.21 (m, 2H), 2.07 (td, J = 4.0, 8.8 Hz, 1H), 1.85 (dd, J = 4.4, 11.8 Hz, 1H), |
| | | 1.68 (s, 2H), 1.58 (s, 1H), 1.40 (d, J = 9.2 Hz, 1H) |
| I-48 | 772.2 | 11.76-11.64 (m, 1H), 10.75 (s, 1H), 8.17-8.08 (m, 1H), 7.69 (d, J = 2.4 Hz, 1H), |
| | | 7.44-7.35 (m, 1H), 7.25-7.18 (m, 1H), 7.01 (d, J = 8.4 Hz, 2H), 6.86 (s, 1H), 6.77 |
| | | (d, J = 6.0 Hz, 2H), 6.27 (s, 1H), 4.70-4.60 (m, 2H), 4.55 (d, J = 13.6 Hz, 1H), |
| | | 4.41-4.29 (m, 2H), 4.12-3.97 (m, 1H), 3.95-3.80 (m, 1H), 3.69 (dd, J = 4.4, 10.4 Hz, |
| | | 1H), 3.64-3.52 (m, 2H), 3.12 (td, J = 6.8, 19.8 Hz, 6H), 2.74 (s, 4H), 2.70-2.54 (m, |
| | | 6H), 2.44 (d, J = 4.4 Hz, 2H), 2.33-2.22 (m, 2H), 2.14-1.95 (m, 2H), 1.63 (s, 3H), |
| | | 1.47 (d, J = 8.8 Hz, 1H), 1.34-1.13 (m, 1H) |
| I-47 | 777.1 | 11.70 (d, J = 12.4 Hz, 1H), 10.87-10.69 (m, 1H), 8.36 (s, 1H), 8.12 (s, 1H), 7.69 (d, |
| | | J = 2.4 Hz, 1H), 7.43-7.35 (m, 1H), 7.25-7.18 (m, 1H), 6.85 (s, 1H), 6.78-6.68 |
| | | (m, 1H), 6.52 (dd, J = 2.4, 14.8 Hz, 1H), 6.39 (d, J = 8.4 Hz, 1H), 6.27 (s, 1H), 5.85 |
| | | (d, J = 7.2 Hz, 1H), 4.71-4.53 (m, 2H), 4.39-4.30 (m, 2H), 4.26 (ddd, J = 4.8, 7.1, |
| | | 11.6 Hz, 1H), 3.64-3.53 (m, 6H), 3.12 (td, J = 6.8, 18.8 Hz, 6H), 2.88-2.78 (m, |
| | | 4H), 2.75 (s, 4H), 2.73-2.64 (m, 2H), 2.58 (d, J = 3.6 Hz, 1H), 2.54 (s, 1H), 2.32- |
| | | 2.23 (m, 2H), 2.08 (td, J = 4.5, 7.9 Hz, 1H), 1.84 (dd, J = 4.0, 11.6 Hz, 1H) |
| I-22 | 778.5 | 10.77 (s, 1H), 8.11 (s, 1H), 7.80-7.71 (m, 1H), 7.69 (s, 1H), 7.60-7.48 (m, 1H), |
| | | 7.40 (d, J = 7.2 Hz, 1H), 6.76 (t, J = 9.2 Hz, 1H), 6.52 (d, J = 14.4 Hz, 1H), 6.39 (d, |
| | | J = 8.0 Hz, 1H), 6.32 (d, J = 10.0 Hz, 1H), 5.98-5.67 (m, 1H), 4.63 (t, J = 6.4 Hz, |
| | | 2H), 4.35 (d, J = 9.2 Hz, 2H), 4.30-4.21 (m, 1H), 3.63-3.55 (m, 5H), 3.28-3.20 |
| | | (m, 3H), 3.18-3.07 (m, 3H), 3.03 (s, 3H), 2.85 (s, 2H), 2.78 (d, J = 5.6 Hz, 2H), 2.75 |
| | | (s, 4H), 2.58 (s, 2H), 2.39-2.18 (m, 3H), 2.13-2.04 (m, 1H), 1.92-1.78 (m, 1H) |
| I-23 | 777.5 | 10.78 (s, 1H), 8.11 (s, 1H), 7.80-7.72 (m, 1H), 7.69 (d, J = 2.4 Hz, 1H), 7.60-7.50 |
| | | (m, 1H), 7.41 (d, J = 7.2 Hz, 1H), 6.87 (dt, J = 3.6, 8.8 Hz, 1H), 6.49-6.37 (m, 2H), |
| | | 6.33 (d, J = 11.2 Hz, 1H), 6.12-5.90 (m, 1H), 4.63 (t, J = 6.4 Hz, 2H), 4.58-4.51 |
| | | (m, 1H), 4.36 (d, J = 11.2 Hz, 2H), 4.32-4.26 (m, 1H), 4.07-3.97 (m, 1H), 3.58 (td, |
| | | J = 5.6, 17.2 Hz, 2H), 3.25 (s, 3H), 3.17-3.09 (m, 3H), 3.03 (s, 3H), 2.92-2.84 (m, |
| | | 1H), 2.80-2.69 (m, 5H), 2.66-2.57 (m, 2H), 2.36-2.19 (m, 3H), 2.11-2.03 (m, |
| | | 1H), 1.91-1.80 (m, 1H), 1.69 (d, J = 1.2 Hz, 2H), 1.63-1.53 (m, |
| | | 1H), 1.48-1.36 (m, 1H) |
| I-24 | 862.5 | 11.07 (s, 1H), 8.11 (s, 1H), 8.01 (d, J = 3.6 Hz, 1H), 7.79-7.71 (m, 1H), 7.69 (d, J = |
| | | 2.0 Hz, 1H), 7.58-7.47 (m, 1H), 7.39 (d, J = 6.8 Hz, 1H), 7.05 (s, 1H), 6.99 (d, J = |
| | | 8.0 Hz, 1H), 6.88 (d, J = 8.4 Hz, 1H), 6.31 (d, J = 12.4 Hz, 1H), 5.33 (dd, J = 5.6, |
| | | 12.8 Hz, 1H), 4.63 (t, J = 6.8 z, 2H), 4.35 (d, J = 8.4 Hz, 2H), 3.62-3.52 (m, 4H), |
| | | 3.46 (d, J = 2.0 Hz, 3H), 3.42-3.38 (m, 2H), 3.31 (s, 3H), 3.26-3.19 (m, 5H), |
| | | 3.17-3.07 (m, 3H), 3.03 (s, 3H), 2.94-2.84 (m, 1H), 2.80 (t, J = 7.2Hz, 2H), 2.76-2.57 |
| | | (m, 5H), 2.45-2.40 (m, 3H), 2.36-2.19 (m, 3H), 2.04-1.94 (m, 1H) |
| I-26 | 950.3 | 11.08 (s, 1H), 8.11 (s, 1H), 8.02 (s, 1H), 7.80-7.71 (m, 1H), 7.69 (s, 1H), 7.60-7.45 |
| | | (m, 1H), 7.39 (d, J = 7.6 Hz, 1H), 7.02-6.84 (m, 3H), 6.32 (d, J = 12.4 Hz, 1H), 5.36 |
| | | (dd, J = 5.2, 12.4 Hz, 1H), 4.63 (t, J = 6.8 Hz, 2H), 4.35 (d, J = 8.4 Hz, 2H), 3.65 (s, |
| | | 2H), 3.60 (s, 1H), 3.56 (s, 4H), 3.50 (d, J = 2.4 Hz, 4H), 3.44 (s, 8H), 3.39 (d, J = 6.0 |
| | | Hz, 2H), 3.29-3.17 (m, 6H), 3.12 (dd, J = 7.2, 14.0 Hz, 4H), 3.03 (s, 2H), 2.94- |
| | | 2.83 (m, 1H), 2.77-2.70 (m, 2H), 2.70-2.56 (m, 2H), 2.43 (t, J = 7.2 Hz, 2H), |
| | | 2.34-2.20 (m, 2H), 2.05-1.91 (m, 1H) |
| I-28 | 828.3 | 10.77 (s, 1H), 8.15 (s, 1H), 8.11 (s, 1H), 7.73 (s, 1H), 7.69 (s, 1H), 7.62-7.47 (m, |
| | | 1H), 7.41 (d, J = 7.6 Hz, 1H), 7.04 (d, J = 8.4 Hz, 2H), 6.86 (d, J = 8.4 Hz, 2H), 6.33 |
| | | (d, J = 12.0 Hz, 1H), 4.63 (t, J = 6.8 Hz, 2H), 4.47-4.30 (m, 3H), 3.96 (d, J = 11.6 |
| | | Hz, 1H), 3.74-3.70 (m, 1H), 3.60-3.55 (m, 2H), 3.25 (s, 4H), 3.20-3.08 (m, 4H), |
| | | 3.05 (s, 6H), 3.01-2.97 (m, 1H), 2.74 (d, J = 5.6 Hz, 3H), 2.63 (s, 2H), 2.58 (s, 4H), |
| | | 2.45 (d, J = 13.1 Hz, 1H), 2.35-2.20 (m, 2H), 2.18-2.06 (m, 1H), 2.05-1.93 (m, |
| | | 1H), 1.80 (s, 2H), 1.39 (d, J = 9.4 Hz, 1H), 1.25 (d, J = 11.6 Hz, 1H) |
| I-30 | 773.5 | 10.80 (s, 1H), 8.11 (s, 1H), 7.80-7.71 (m, 1H), 7.69 (d, J = 1.6 Hz, 1H), 7.61-7.47 |
| | | (m, 1H), 7.41 (d, J = 7.2 Hz, 1H), 7.13 (s, 1H), 6.92-6.57 (m, 2H), 6.51 (s, 1H), 6.33 |
| | | (d, J = 11.6 Hz, 1H), 4.63 (t, J = 6.8 Hz, 2H), 4.58-4.49 (m, 1H), 4.36 (d, J = 8.8 |
| | | Hz, 2H), 4.01 (d, J = 12.0 Hz, 1H), 3.94-3.83 (m, 1H), 3.75 (dd, J = 4.0, 10.4 Hz, |
| | | 1H), 3.58 (td, J = 5.6, 17.2 Hz, 2H), 3.25 (s, 3H), 3.21-3.07 (m, 4H), 3.03 (s, 3H), |
| | | 2.74 (s, 4H), 2.68-2.59 (m, 4H), 2.45 (d, J = 4.4 Hz, 1H), 2.32 (s, 1H), 2.27-2.12 |
| | | (m, 2H), 2.08-1.96 (m, 1H), 1.65 (s, 3H), 1.48 (d, J = 10.8 Hz, 1H) |
| I-31 | 773.2 | 10.76 (s, 1H), 8.11 (s, 1H), 7.81-7.72 (m, 1H), 7.69 (d, J = 1.2 Hz, 1H), 7.61-7.47 |
| | | (m, 1H), 7.41 (d, J = 7.2 Hz, 1H), 7.01 (d, J = 7.2 Hz, 2H), 6.79 (s, 2H), 6.33 (d, J = |
| | | 11.8 Hz, 1H), 4.63 (t, J = 6.8 Hz, 2H), 4.54 (d, J = 12.4 Hz, 1H), 4.36 (d, J = 9.6 Hz, |
| | | 2H), 4.07-3.96 (m, 1H), 3.94-3.82 (m, 1H), 3.70 (dd, J = 4.0, 10.2 Hz, 1H), 3.58 |
| | | (td, J = 5.2, 16.8 Hz, 2H), 3.25 (s, 3H), 3.19-3.07 (m, 4H), 3.06-2.96 (m, 3H), 2.74 |
| | | (s, 4H), 2.66-2.58 (m, 4H), 2.44 (d, J = 4.4 Hz, 1H), 2.32 (d, J = 1.2 Hz, 1H), 2.24 |
| | | (s, 1H), 2.17-2.04 (m, 1H), 2.04-1.94 (m, 1H), 1.64 (s, 3H), 1.55-1.41 (m, 1H) |
| 1-99 | 949.6 | 11.68 (d, J = 12.4 Hz, 1H), 11.07 (s, 1H), 8.11 (s, 1H), 8.05-7.98 (m, 1H), 7.69 (d, |
| | | J = 2.8 Hz, 1H), 7.42-7.35 (m, 1H), 7.25-7.22 (m, 1H), 7.11 (s, 1H), 7.06 (s, 1H), |
| | | 6.98 (d, J = 1.2 Hz, 1H), 6.89 (d, J = 7.6 Hz, 1H), 6.81 (s, 1H), 6.26 (s, 1H), 5.33 (dd, |
| | | J = 5.6, 12.8 Hz, 1H), 4.72-4.56 (m, 2H), 4.39-4.27 (m, 2H), 3.62-3.53 (m, 5H), |
| | | 3.49 (s, 3H), 3.47-3.45 (m, 5H), 3.44 (s, 3H), 3.41-3.38 (m, 6H), 3.25-3.22 (m, |
| | | 3H), 3.17-3.06 (m, 5H), 2.86-2.78 (m, 3H), 2.74-2.68 (m, 3H), 2.46-2.42 (m, |
| | | 2H), 2.35-2.29 (m, 2H), 2.27-2.21 (m, 1H), 2.04-1.94 (m, 2H) |
| I-100 | 827.2 | 11.78-11.65 (m, 1H), 10.77 (s, 1H), 8.44 (s, 1H), 8.11 (s, 1H), 7.69 (s, 1H), 7.44- |
| | | 7.35 (m, 1H), 7.27-7.17 (m, 1H), 7.04 (d, J = 8.4 Hz, 2H), 6.84 (d, J = 8.0 Hz, 3H), |
| | | 6.28 (s, 1H), 4.68-4.57 (m, 2H), 4.44 (d, J = 11.4 Hz, 1H), 4.39-4.29 (m, 2H), |
| | | 4.05-3.93 (m, 1H), 3.72 (dd, J = 4.8, 10.8 Hz, 1H), 3.61-3.54 (m, 2H), 3.17-3.06 (m, |
| | | 6H), 3.01 (s, 6H), 2.82-2.76 (m, 1H), 2.75-2.53 (m, 8H), 2.45-2.42 (m, 2H), 2.32 |
| | | (d, J = 2.0 Hz, 4H), 2.14-2.06 (m, 1H), 2.00 (td, J = 4.8, 8.0 Hz, 1H), 1.81-1.72 |
| | | (m, 2H), 1.42-1.32 (m, 1H), 1.25-1.18 (m, 1H) |
| I-101 | 831.2 | 11.53-11.41 (m. 1H), 10.78 (s, 1H), 8.11 (s, 1H), 7.72-7.65 (m, 1H), 7.27-7.21 |
| | | (m, 1H), 7.15 (t, J = 7.6 Hz, 1H), 7.03-6.96 (m, 1H), 6.90 (d, J = 2.0 Hz, 1H), 6.84- |
| | | 6.74 (m, 2H), 6.61 (d, J = 7.6 Hz, 1H), 6.29-6.22 (m, 1H), 4.63 (t, J = 6.8 Hz, 2H), |
| | | 4.42-4.30 (m, 3H), 3.84 (br d, J = 13.2 Hz, 1H), 3.75 (dd, J = 5.2, 11.2 Hz, 1H), |
| | | 3.62 (br t, J = 5.6 Hz, 1H), 3.56 (br t, J = 5.6 Hz, 2H), 3.16-3.02 (m, 9H), 3.00- |
| | | 2.83 (m, 4H), 2.69-2.54 (m, 6H), 2.49-2.39 (m, 3H), 2.33 (br d, J = 3.6 Hz, 3H), |
| | | 2.28-2.13 (m, 2H), 2.06-1.98 (m, 1H), 1.80-1.66 (m, 4H), 1.60-1.50 (m, 2H), |
| | | 1.32-1.12 (m, 2H) |
| I-102 | 772.5 | 11.80-11.59 (m, 1H), 10.95-10.56 (m, 1H), 8.42 (s, 1H), 8.12 (s, 1H), 7.73-7.64 |
| | | (m, 1H), 7.43-7.35 (m, 1H), 7.24-7.20 (m, 1H), 7.11 (t, J = 8.0 Hz, 1H), 6.86 (s, |
| | | 1H), 6.77-6.68 (m, 1H), 6.64 (s, 1H), 6.46 (d, J = 7.6 Hz, 1H), 6.27 (s, 1H), 4.69- |
| | | 4.59 (m, 2H), 4.55 (d, J = 11.2 Hz, 1H), 4.41-4.28 (m, 2H), 4.12-3.97 (m, 1H), |
| | | 3.94-3.83 (m, 1H), 3.73 (dd, J = 4.8, 10.8 Hz, 1H), 3.61-3.54 (m, 2H), 3.18-3.04 |
| | | (m, 6H), 2.74 (s, 4H), 2.70-2.58 (m, 4H), 2.57 (s, 3H), 2.45 (d, J = 4.4 Hz, 1H), 2.32 |
| | | (d, J = 1.6 Hz, 2H), 2.27-2.14 (m, 2H), 2.03 (dt, J = 4.8, 9.2 Hz, 1H), 1.64 (s, 3H), |
| | | 1.48 (d, J = 9.6 Hz, 1H) |
| I-103 | 776.3 | 11.55-11.40 (m, 1H), 10.78 (s, 1H), 8.15-8.10 (m, 1H), 7.74-7.67 (m, 1H), 7.27- |
| | | 7.23 (m, 1H), 7.12 (t, J = 8.0 Hz, 1H), 7.04-6.98 (m, 1H), 6.92 (d, J = 2.0 Hz, 1H), |
| | | 6.72 (dd, J = 2.0, 8.4 Hz, 1H), 6.66 (s, 1H), 6.47 (d, J = 7.6 Hz, 1H), 6.31-6.22 (m, |
| | | 1H), 4.64 (t, J = 6.8 Hz, 2H), 4.50 (br d, J = 13.2 Hz, 1H), 4.41-4.32 (m, 2H), |
| | | 3.96-3.82 (m, 2H), 3.74 (dd, J = 4.8, 11.2 Hz, 1H), 3.64-3.55 (m, 2H), 3.20-3.00 (m, |
| | | 6H), 2.88 (br t, J = 7.6 Hz, 2H), 2.70-2.52 (m, 7H), 2.50-2.31 (m, 5H), 2.28-2.13 |
| | | (m, 2H), 2.03 (qd, J = 4.8, 13.2 Hz, 1H), 1.73 (td, J = 7.6, 14.4 Hz, 2H), 1.65-1.51 |
| | | (m, 5H), 1.50-1.41 (m, 1H) |
| I-4 | 1011.5 | 8.98 (s, 1H), 8.54 (t, J = 6.0 Hz, 1H), 8.19-8.03 (m, 1H), 7.90-7.76 (m, 1H), |
| | | 7.74-7.49 (m, 5H), 7.39 (q, J = 8.0 Hz, 5H), 6.69 (d, J = 8.4 Hz, 2H), 6.46-6.25 (m, 1H), |
| | | 4.64 (t, J = 6.8 Hz, 2H), 4.58-4.50 (m, 1H), 4.47-4.37 (m, 4H), 4.34 (s, 1H), 4.21 |
| | | (dd, J = 5.6, 15.9 Hz, 1H), 3.68-3.54 (m, 5H), 3.18-3.07 (m, 3H), 3.04-2.91 (m, |
| | | 2H), 2.74 (s, 3H), 2.43 (s, 3H), 2.37-2.21 (m, 4H), 2.11-1.81 (m, 3H), 1.73-1.33 |
| | | (m, 5H), 1.32-1.08 (m, 3H), 0.91 (s, 9H) |
| I-27 | 862.4 | 11.08 (s, 1H), 8.11 (s, 1H), 8.01 (d, J = 3.2 Hz, 1H), 7.78-7.70 (m, 1H), 7.69 (d, J = |
| | | 2.0 Hz, 1H), 7.58-7.48 (m, 1H), 7.39 (d, J = 7.6 Hz, 1H), 7.01-6.92 (m, 2H), |
| | | 6.91-6.86 (m, 1H), 6.31 (d, J = 13.2 Hz, 1H), 5.36 (dd, J = 5.2, 12.4 Hz, 1H), 4.63 |
| | | (t, J = 6.8 Hz, 2H), 4.35 (d, J = 9.2 Hz, 2H), 3.65-3.57 (m, 3H), 3.55 (s, 3H), 3.46 (s, |
| | | 3H), 3.40-3.36 (m, 3H), 3.26-3.17 (m, 5H), 3.14-3.09 (m, 3H), 3.02 (s, 3H), |
| | | 2.93-2.83 (m, 1H), 2.76-2.69 (m, 3H), 2.69-2.57 (m, 2H), 2.46-2.38 |
| | | (m, 3H), 2.33-2.22 (m, 2H), 2.05-1.93 (m, 1H) |
| I-25 | 950.3 | 11.07 (s, 1H), 8.11 (s, 1H), 8.02 (s, 1H), 7.80-7.70 (m, 1H), 7.69 (d, J = 1.6 Hz, 1H), |
| | | 7.59-7.48 (m, 1H), 7.39 (d, J = 7.2 Hz, 1H), 7.06 (s, 1H), 6.99 (d, J = 8.0 Hz, 1H), |
| | | 6.89 (d, J = 8.0 Hz, 1H), 6.42-6.24 (m, 1H), 5.33 (dd, J = 5.2, 12.8 Hz, 1H), 4.63 (t, |
| | | J = 6.8 Hz, 2H), 4.35 (d, J = 8.8 Hz, 2H), 3.61-3.55 (m, 3H), 3.50 (s, 3H), 3.47- |
| | | 3.44 (m, 6H), 3.40 (t, J = 6.0 Hz, 3H), 3.31 (s, 6H), 3.25-3.21 (m, 4H), 3.16-3.08 |
| | | (m, 3H), 3.03 (s, 2H), 2.92-2.80 (m, 3H), 2.74-2.70 (m, 2H), 2.68-2.57 (m, 2H), |
| | | 2.43 (t, J = 7.2 Hz, 3H), 2.32-2.23 (m, 2H), 2.04-1.94 (m, 1H) |
| I-21 | 492.9^{b} | 11.39-10.92 (br, s, 1H), 8.45 (d, J = 2.4 Hz, 1H), 8.26-8.06 (m, 1H), 7.83 (dd, J = |
| | | 2.4, 8.8 Hz, 1H), 7.75-7.50 (m, 5H), 7.44 (s, 1H), 7.37 (d, J = 10.4 Hz, 1H), 7.27 |
| | | (d, J = 8.8 Hz, 1H), 7.15 (d, J = 8.4 Hz, 1H), 6.89 (d, J = 8.8 Hz, 1H), 6.67 (d, J = |
| | | 8.8 Hz, 2H), 6.36 (s, J = 10.8 Hz, 1H), 5.96 (br s, 1H), 5.38 (br dd, J = 5.2, 12.8 Hz, |
| | | 1H), 4.64 (t, J = 6.8 Hz, 2H), 4.51-4.22 (dd, 6H), 3.60 (td, J = 5.6, 19.0 Hz, 2H), |
| | | 3.39 (m, 3H), 3.18-3.05 (m, 4H), 2.93-2.75 (m, 4H), 2.72 (br d, J = 4.3 Hz, 3H), |
| | | 2.68-2.59 (s, 1H), 2.37-2.21 (t, 2H), 2.10-1.95 (m, 1H), 1.76 (br t, |
| | | J = 14.7 Hz, 4H), 1.64-1.41 (m, 2H), 1.38-0.98 (m, 8H) |
| I-109 | 776.3 | 11.53-11.41 (m, 1H), 10.92-10.53 (m, 1H), 8.41 (s, 1H), 8.11 (s, 1H), 7.69 (s, 1H), |
| | | 7.26-7.21 (m, 1H), 7.00 (br d, J = 8.8 Hz, 3H), 6.92-6.86 (m, 1H), 6.77 (br d, J = |
| | | 8.8 Hz, 2H), 6.24 (br s, 1H), 4.63 (br t, J = 6.8 Hz, 2H), 4.49 (br d, J = 11.6 Hz, 1H), |
| | | 4.41-4.30 (m, 2H), 3.96-3.79 (m, 2H), 3.69 (br dd, J = 4.8, 10.8 Hz, 1H), 3.61 (br |
| | | t, J = 5.6 Hz, 1H), 3.56 (br t, J = 5.2 Hz, 2H), 3.17-3.02 (m, 6H), 2.87 (br t, J = 7.2 |
| | | Hz, 2H), 2.70-2.54 (m, 5H), 2.48-2.20 (m, 6H), 2.18-2.05 (m, 2H), 2.04-1.94 |
| | | (m, 1H), 1.76-1.67 (m, 2H), 1.63-1.50 (m, 5H), 1.48-1.39 (m, 1H) |
| I-6 | 933.5 | δ 11.57-11.49 (m, 1H), 8.98 (s, 1H), 8.57 (t, J = 6.0 Hz, 1H), 8.13 (s, 1H), 7.86 (br |
| | | d, J = 9.8 Hz, 1H), 7.72-7.67 (m, 1H), 7.61-7.55 (m, 1H), 7.44-7.35 (m, 5H), |
| | | 7.23-7.16 (m, 1H), 6.88-6.69 (m, 1H), 6.27 (br d, J = 1.6 Hz, 1H), 5.13 (br d, J = 3.6 |
| | | Hz, 1H), 4.67-4.60 (m, 2H), 4.54 (d, J = 9.6 Hz, 1H), 4.47-4.33 (m, 5H), 4.21 (dd, |
| | | J = 5.4, 16.0 Hz, 1H), 3.68-3.55 (m, 5H), 3.17-3.06 (m, 3H), 2.44 (s, 3H), 2.28- |
| | | 2.21 (m, 2H), 2.15-1.98 (m, 3H), 1.89 (ddd, J = 4.4, 8.4, 12.8 Hz, 1H), 1.65-1.38 |
| | | (m, 5H), 1.33-1.20 (m, 7H), 0.94-0.89 (m, 9H) |
| I-40 | 781.5 | 10.78 (s, 1H), 8.25-7.92 (s, 1H), 7.69 (d, J = 2.4 Hz, 1H), 7.63-7.52 (d, 1H), 7.45- |
| | | 7.33 (m, 2H), 6.92 (t, J = 8.4 Hz, 1H), 6.49-6.39 (d, 2H), 6.29 (br d, J = 16.4 Hz, |
| | | 1H), 6.14-5.80 (br s, 1H), 4.63 (t, J = 6.8 Hz, 2H), 4.49 (br d, J = 12.0 Hz, 1H), 4.41- |
| | | 4.25 (m, 3H), 3.90 (br d, J = 12.0 Hz, 1H), 3.62-3.55 (m, 1H), 3.26 (br s, 3H), 3.17- |
| | | 3.01 (m, 6H), 2.92-2.66 (m, 4H), 2.61-2.52 (m, 2H), 2.40-2.21 (m, 4H), 2.12- |
| | | 2.01 (m, 1H), 1.93-1.72 (m, 3H), 1.65 (br s, 2H), 1.59-1.33 (m, 4H) |
| I-112 | 814.5 | 11.16-11.06 (m, 1H), 8.16-8.07 (m, 1H), 7.82-7.73 (m, 1H), 7.71-7.64 (m, 1H), |
| | | 7.63-7.51 (m, 1H), 7.44 (s, 1H), 6.99-6.83 (m, 3H), 6.38-6.30 (m, 1H), 5.41 (s, |
| | | 1H), 4.67-4.56 (m, 3H), 4.36 (br d, J = 9.2 Hz, 2H), 4.12-4.03 (m, 1H), 3.63-3.60 |
| | | (m, 3H), 3.59-3.52 (m, 2H), 3.29-3.21 (m, 6H), 3.17-3.08 (m, 3H), 3.07-2.98 |
| | | (m, 3H), 2.78 (br d, J = 6.0 Hz, 3H), 2.74-2.71 (m, 1H), 2.66 (br d, J = 12.8 Hz, |
| | | 2H), 2.34-2.31 (m, 1H), 2.24 (br d, J = 3.2 Hz, 1H), 2.02-1.96 (m, 1H), 1.89-1.80 |
| | | (m, 2H), 1.77-1.67 (m, 1H), 1.60-1.48 (m, 1H) |
| I-113 | 879.9 | 11.11 (s, 1H), 8.43 (s, 1H), 8.21-8.05 (m, 1H), 7.87 (dd, J = 1.6, 8.4 Hz, 1H), 7.81- |
| | | 7.72 (m, 1H), 7.69 (d, J = 2.0 Hz, 1H), 7.65-7.61 (m, 1H), 7.60-7.50 (m, 1H), |
| | | 7.45 (s, 1H), 7.36-7.24 (m, 2H), 7.15 (d, J = 8.4 Hz, 1H), 7.01-6.86 (m, 1H), 6.39- |
| | | 6.16 (m, 1H), 5.39 (dd, J = 5.6, 12.8 Hz, 1H), 4.63 (t, J = 6.8 Hz, 2H), 4.48-4.12 |
| | | (m, 6H), 3.59 (td, J = 5.6, 18.0 Hz, 2H), 3.39 (s, 3H), 3.13 (td, J = 6.8, 18.4 Hz, 3H), |
| | | 2.96-2.60 (m, 6H), 2.39-2.19 (m, 2H), 2.10-1.97 (m, 1H), 1.83-1.68 (m, 4H), |
| | | 1.59-1.42 (m, 2H), 1.28 (s, 4H), 1.20-1.02 (m, 4H) |
| I-114 | 888.4 | 11.06 (s, 1H), 8.11 (s, 1H), 7.69 (d, J = 1.6 Hz, 1H), 7.64-7.56 (m, 1H), 7.44-7.34 |
| | | (m, 2H), 6.93 (d, J = 8.4 Hz, 1H), 6.83 (d, J = 2.0 Hz, 1H), 6.62 (dd, J = 2.0, 8.4 Hz, |
| | | 1H), 6.30 (d, J = 14.4 Hz, 1H), 5.29 (dd, J = 5.2, 12.8 Hz, 1H), 4.69-4.61 (m, 2H), |
| | | 4.59 (s, 1H), 4.36 (d, J = 15.2 Hz, 2H), 4.05 (d, J = 12.8 Hz, 1H), 3.58 (td, J = 5.6, |
| | | 19.2 Hz, 2H), 3.30 (s, 3H), 3.27-3.17 (m, 6H), 3.17-2.98 (m, 10H), 2.93-2.57 (m, |
| | | 8H), 2.41 (t, J = 7.2 Hz, 2H), 2.33-2.22 (m, 2H), 2.02-1.95 (m, 1H), 1.92-1.70 |
| | | (m, 6H) |
| I-115 | 815.2 | 11.25-10.79 (m, 1H), 8.11 (s, 1H), 7.82-7.66 (m, 2H), 7.59-7.48 (m, 1H), 7.40 (d, |
| | | J = 6.4 Hz, 1H), 6.94 (d, J = 8.8 Hz, 1H), 6.85 (d, J = 1.2 Hz, 1H), 6.62 (dd, J = 1.6, |
| | | 8.4 Hz, 1H), 6.42-6.18 (m, 1H), 5.42-5.15 (m, 1H), 4.71-4.56 (m, 2H), 4.34 (d, |
| | | J = 11.2 Hz, 2H), 3.65 (s, 4H), 3.60-3.51 (m, 2H), 3.29 (s, 3H), 3.24 (s, 3H), 3.18- |
| | | 3.07 (m, 5H), 3.04 (d, J = 5.2 Hz, 4H), 2.93-2.84 (m, 1H), 2.77 (s, 3H), 2.76-2.56 |
| | | (m, 3H), 2.34-2.17 (m, 2H), 2.05-1.88 (m, 1H) |
| I-116 | 815.5 | 11.09 (s, 1H), 8.11 (s, 1H), 7.84-7.72 (m, 1H), 7.69 (d, J = 2.0 Hz, 1H), 7.61-7.48 |
| | | (m, 1H), 7.41 (d, J = 7.2 Hz, 1H), 7.00-6.76 (m, 3H), 6.43-6.22 (m, 1H), 5.35 (dd, |
| | | J = 5.2, 12.4 Hz, 1H), 4.62 (dt, J = 3.2, 6.8 Hz, 2H), 4.55-4.41 (m, 1H), 4.36 (d, J = |
| | | 10.0 Hz, 2H), 4.12-3.88 (m, 1H), 3.63 (s, 3H), 3.58 (td, J = 5.6, 16.8 Hz, 2H), 3.25 |
| | | (s, 3H), 3.18-3.08 (m, 3H), 3.08-2.98 (m, 4H), 2.97-2.54 (m, 11H), 2.36-2.19 |
| | | (m, 2H), 2.05-1.93 (m, 1H) |
| I-32 | 782.4 | 10.77 (s, 1H), 8.11 (s, 1H), 7.69 (d, J = 2.0 Hz, 1H), 7.64-7.53 (m, 1H), 7.46-7.31 |
| | | (m, 2H), 6.80 (t, J = 9.2 Hz, 1H), 6.52 (dd, J = 2.4, 14.8 Hz, 1H), 6.42 (dd, J = 2.0, |
| | | 8.8 Hz, 1H), 6.29 (br d, J = 16.4 Hz, 1H), 5.85 (d, J = 7.6 Hz, 1H), 4.63 (t, J = 6.8 |
| | | Hz, 2H), 4.37 (br d, J = 13.2 Hz, 2H), 4.26 (ddd, J = 4.8, 7.6, 11.8 Hz, 1H), 3.67- |
| | | 3.46 (m, 6H), 3.26 (br s, 3H), 3.12 (td, J = 6.8, 20.0 Hz, 3H), 3.02 (br s, 3H), 2.90 (br |
| | | t, J = 7.2 Hz, 2H), 2.81-2.65 (m, 5H), 2.57 (td, J = 4.0, 17.4 Hz, 2H), 2.37 (br t, J = |
| | | 7.2 Hz, 2H), 2.34-2.19 (m, 2H), 2.15-2.03 (m, 1H), 1.85 (br dd, J = 4.4, 12.4 Hz, |
| | | 1H), 1.79-1.68 (m, 2H), 1.61-1.50 (m, 2H) |
| I-118 | 888.4 | 11.08 (s, 1H), 8.22 (s, 1H), 8.11 (s, 1H), 7.76-7.51 (m, 2H), 7.49-7.28 (m, 2H), |
| | | 7.06-6.79 (m, 3H), 6.30 (d, J = 15.6 Hz, 1H), 5.35 (dd, J = 5.4, 12.6 Hz, 1H), 4.63 |
| | | (t, J = 6.8 Hz, 3H), 4.36 (d, J = 12.8 Hz, 2H), 4.06 (d, J = 13.2 Hz, 1H), 3.62 (s, 3H), |
| | | 3.55 (s, 2H), 3.33-3.22 (m, 6H), 3.19-2.98 (m, 8H), 2.97-2.80 (m, 6H), 2.75-2.55 |
| | | (m, 4H), 2.40 (d, J = 4.4 Hz, 2H), 2.30 (d, J = 2.2 Hz, 2H), 2.04 - 1.96 (m, 1H), 1.94-1.63 (m, 6H) |
| I-120 | 780.6 | 10.95 (s, 1H), 8.11 (d, J = 2.4 Hz, 1H), 7.74-7.66 (m, 1H), 7.33 (d, J = 12.0 Hz, 1H), |
| | | 7.05 (s, 1H), 7.00 (t, J = 8.0 Hz, 1H), 6.52-6.43 (m, 3H), 6.28-6.22 (m, 1H), 6.03 |
| | | (d, J = 8.0 Hz, 1H), 4.63 (t, J = 6.8 Hz, 2H), 4.55-4.49 (m, 1H), 4.34 (d, J = 7.6 Hz, |
| | | 2H), 4.31 (s, 2H), 4.30-4.26 (m, 1H), 4.25-4.21 (m, 1H), 4.18 (t, J = 7.2 Hz, 1H), |
| | | 3.97-3.88 (m, 1H), 3.79-3.70 (m, 1H), 3.63-3.58 (m, 1H), 3.57-3.54 (m, 1H), |
| | | 3.27 (d, J = 4.0 Hz, 6H), 3.14 (d, J = 6.4 Hz, 2H), 3.11-3.07 (m, 2H), 3.01 (s, 2H), |
| | | 2.35-2.27 (m, 2H), 2.25-2.21 (m, 1H), 2.07 (dd, J = 4.0, 8.4 Hz, 1H), 1.89-1.82 |
| | | (m, 1H), 1.75-1.67 (m, 2H), 1.57-1.44 (m, 2H) |
| I-122 | 887.4 | 1.3-1.5 (m, 2 H) 1.8-1.9 (m, 2 H) 1.9-2.0 (m, 2 H) 2.2-2.3 (m, 1 H) 2.3 (d, J = 3.2 |
| | | Hz, 1 H) 2.4 (t, J = 6.4 Hz, 2 H) 2.6-2.7 (m, 2 H) 2.9-2.9 (m, 1 H) 3.0-3.0 (m, 2 H) |
| | | 3.1-3.1 (m, 4 H) 3.3-3.3 (m, 6 H) 3.3 (s, 4 H) 3.5-3.6 (m, 4 H) 3.9-3.9 (m, 2 H) |
| | | 4.2 (t, J-6.4 Hz, 2 H) 4.3-4.4 (m, 4 H) 4.6-4.7 (m, 2 H) 5.3-5.4 (m, 1 H) 6.2-6.3 |
| | | (m, 1 H) 6.4-6.6 (m, 1 H) 6.9-7.0 (m, 1 H) 7.0-7.1 (m, 3 H) 7.3-7.4 (m, 1 H) 7.7 |
| | | (d, J = 1.6 Hz, 1 H) 8.1 (s, 1 H) 11.1 (s, 1 H) |
| I-124 | 836.4^{d} | (methanol-d₄) 8.05-8.00 (m, 1H), 7.71 (br d, J = 6.4 Hz, 2H), 7.56 (s, 1H), 7.37 (d, |
| | | J = 5.6 Hz, 1H), 7.00-6.94 (m, 1H), 6.87-6.80 (m, 1H), 6.76-6.66 (m, 1H), 6.27- |
| | | 6.19 (m, 1H), 5.37-5.28 (m, 1H), 4.60 (s, 3H), 4.40-4.40 (m, 1H), 4.32-4.24 (m, |
| | | 2H), 3.70 (br t, J = 6.4 Hz, 1H), 3.73-3.67 (m, 1H), 3.62 (t, J = 5.6 Hz, 1H), 3.22- |
| | | 3.12 (m, 8H), 3.23-3.12 (m, 1H), 3.03-2.67 (m, 12H), 2.67-2.65 (m, 1H), 2.28- |
| | | 2.28 (m, 1H), 2.30-2.27 (m, 1H), 2.20-2.14 (m, 1H), 2.12 (br d, J = 2.0 Hz, 1H), |
| | | 1.96-1.87 (m, 2H), 1.80-1.72 (m, 1H), 1.70-1.69 (m, 1H), 1.67-1.57 (m, 1H), |
| | | 1.37-1.31 (m, 1H), 1.29-1.29 (m, 1H). |
| I-129 | 888.4 | 10.79 (s, 1H), 8.15-8.11 (m, 1H), 7.69 (d, J = 2.8 Hz, 1H), 7.40-7.20 (m, 2H), 7.11 |
| | | (d, J = 8.8 Hz, 2H), 6.95-6.82 (m, 3H), 6.32-6.15 (m, 1H), 4.63 (t, J = 6.8 Hz, 2H), |
| | | 4.45-4.30 (m, 4H), 4.00 (br d, J = 12.8 Hz, 1H), 3.89-3.81 (m, 2H), 3.77 (dd, J = |
| | | 4.8, 11.2 Hz, 1H), 3.62-3.55 (m, 2H), 3.31-3.24 (m, 6H), 3.15-3.01 (m, 6H), 2.90- |
| | | 2.83 (m, 2H), 2.72 (br d, J = 2.4 Hz, 2H), 2.66-2.58 (m, 1H), 2.23 (br s, 6H), 2.17- |
| | | 2.11 (m, 1H), 2.03-1.91 (m, 3H), 1.81-1.59 (m, 9H), 1.09-0.86 (m, 2H). |
| I-130 | 899.1 | 11.08 (s, 1H), 8.11 (d, J = 2.4 Hz, 1H), 7.69 (d, J = 3.0 Hz, 1H), 7.35 (d, J = 12.4 Hz, |
| | | 1H), 7.25 (d, J = 20.0 Hz, 1H), 7.01-6.97 (m, 2H), 6.91-6.83 (m, 2H), 6.26 (br d, |
| | | J = 13.6 Hz, 1H), 5.33 (dd, J = 5.2, 12.6 Hz, 1H), 4.63 (t, J = 6.8 Hz, 2H), 4.34 (br d, |
| | | J = 13.6 Hz, 2H), 3.87-3.81 (m, 2H), 3.62-3.54 (m, 2H), 3.32 (s, 4H), 3.27 (br s, |
| | | 3H), 3.17-3.07 (m, 3H), 3.02 (br s, 3H), 2.96-2.84 (m, 4H), 2.82 (br d, J = 3.2 Hz, |
| | | 1H), 2.72 (br d, J = 7.2 Hz, 2H), 2.69-2.58 (m, 2H), 2.32 (br s, 2H), 2.23 (br s, 1H), |
| | | 2.03-1.97 (m, 1H), 1.93-1.87 (m, 2H), 1.74 (br s, 4H), 1.59-1.39 (m, 7H) |
| I-131 | 900.0 | 11.04 (s, 1H), 8.12 (s, 1H), 7.69 (d, J = 2.4 Hz, 1H), 7.36 (d, J = 12.4 Hz, 1H), |
| | | 7.30-7.21 (m, 1H), 6.95-6.86 (m, 2H), 6.41 (br s, 1H), 6.30-6.20 (m, 2H), 5.26 (br dd, |
| | | J = 5.2, 12.8 Hz, 1H), 4.63 (t, J = 6.98 Hz, 2H), 4.35 (br d, J = 14.4 Hz, 2H), 3.86 (br |
| | | dd, J = 3.2, 7.9 Hz, 2H), 3.63-3.54 (m, 5H), 3.32 (br s, 2H), 3.29 (s, 6H), 3.18 (br s, |
| | | 2H), 3.15 (br d, J = 7.2 Hz, 1H), 3.10 (br t, J = 6.8 Hz, 2H), 3.03 (br s, 2H), 2.94- |
| | | 2.85 (m, 4H), 2.70-2.58 (m, 2H), 2.38-2.27 (m, 2H), 2.23 (br d, J = 4.4 Hz, 1H), |
| | | 2.00-1.95 (m, 1H), 1.91 (br t, J = 6.4 Hz, 2H), 1.78 (br s, 4H), 1.59 (br d, J = 1.6 |
| | | Hz, 2H), 1.51 (br s, 2H) |
| I-132 | 903.5 | 11.09 (s, 1H), 8.12 (s, 1H), 7.70 (br s, 1H), 7.36 (d, J = 12.4 Hz, 1H), 7.29-7.22 (m, |
| | | 1H), 7.05-6.99 (m, 2H), 6.87 (br s, 2H), 6.27 (br d, J = 13.2 Hz, 1H), 5.35 (br s, |
| | | 1H), 4.63 (br t, J = 6.8 Hz, 2H), 4.35 (br d, J = 14.4 Hz, 2H), 3.90-3.76 (m, 4H), |
| | | 3.62-3.55 (m, 2H), 3.51 (br s, 1H), 3.44 (br t, J = 6.0 Hz, 2H), 3.33-3.27 (m, 6H), |
| | | 3.16-3.00 (m, 7H), 2.92-2.82 (m, 4H), 2.72-2.63 (m, 4H), 2.33-2.22 (m, 2H), |
| | | 2.03-1.97 (m, 1H), 1.86-1.74 (m, 8H), 1.47-1.31 (m, 2H) |
| I-137 | 899.5 | 11.13 (s, 1H), 8.11 (d, J = 2.4 Hz, 1H), 7.69 (d, J = 2.8 Hz, 1H), 7.36 (d, J = 12.4 Hz, |
| | | 1H), 7.29-7.21 (m, 1H), 7.18 (d, J = 7.6 Hz, 1H), 7.13 (d, J = 7.2 Hz, 1H), 7.06- |
| | | 7.00 (m, 1H), 6.89 (br d, J = 15.6 Hz, 1H), 6.33-6.20 (m, 1H), 5.40 (dd, J = 5.6, |
| | | 12.6 Hz, 1H), 4.63 (br t, J = 6.8 Hz, 2H), 4.53 (s, 2H), 4.39-4.30 (m, 2H), 3.93- |
| | | 3.78 (m, 5H), 3.65 (s, 2H), 3.63-3.59 (m, 1H), 3.56 (br t, J = 5.6 Hz, 1H), 3.30- |
| | | 3.24 (m, 4H), 3.19-3.07 (m, 4H), 3.02 (br s, 2H), 2.94-2.84 (m, 4H), 2.77-2.63 |
| | | (m, 2H), 2.45 (br dd, J = 1.2, 2.4 Hz, 1H), 2.33-2.22 (m, 2H), 2.06-2.00 (m, 1H), |
| | | 1.98-1.84 (m, 2H), 1.76 (br s, 4H), 1.54-1.35 (m, 2H) |
| I-138 | 394.4^{b} | 10.75 (s, 1H), 8.11 (s, 1H), 7.79-7.66 (m, 2H), 7.51 (s, 1H), 7.59 (s, 1H), 7.41 (d, |
| | | J = 7.5 Hz, 1H), 6.96 (d, J = 8.5 Hz, 2H), 6.58 (br d, J = 8.5 Hz, 2H), 6.37-6.30 (m, |
| | | 1H), 4.63 (br t, J = 6.8 Hz, 2H), 4.46-4.39 (m, 1H), 4.36 (br d, J = 10.0 Hz, 2H), |
| | | 3.93 (br d, J = 14.0 Hz, 1H), 3.70-3.63 (m, 1H), 3.58 (td, J = 5.5, 16.5 Hz, 2H), 3.25 |
| | | (br s, 3H), 3.18-3.07 (m, 5H), 3.06-2.98 (m, 3H), 2.98-2.91 (m, 1H), 2.86 (s, 3H), |
| | | 2.73 (br s, 2H), 2.70-2.63 (m, 2H), 2.62-2.56 (m, 1H), 2.47-2.40 (m, 1H), 2.32 |
| | | (br s, 1H), 2.24 (br s, 1H), 2.15-2.04 (m, 1H), 2.03-1.87 (m, 2H), 1.69-1.57 (m, |
| | | 2H), 1.28-1.12 (m, 1H), 1.07-0.94 (m, 1H) |
| I-139 | 882.5 | 10.78 (s, 1H), 8.13 (s, 1H), 8.11 (s, 1H), 7.69 (d, J = 2.0 Hz, 1H), 7.36-7.30 (m, 1H), |
| | | 7.11-7.04 (m, 1H), 6.82 (br t, J = 9.2 Hz, 1H), 6.53-6.41 (m, 3H), 6.27-6.20 (m, |
| | | 1H), 5.81 (br d, J = 7.6 Hz, 1H), 5.16-4.96 (m, 1H), 4.63 (br t, J = 6.8 Hz, 2H), |
| | | 4.37-4.21 (m, 6H), 4.19-4.09 (m, 2H), 4.00-3.82 (m, 2H), 3.61-3.54 (m, 2H), 3.26 |
| | | (br s, 6H), 3.15-3.06 (m, 4H), 3.01 (br s, 2H), 2.86 (br s, 4H), 2.73 (ddd, J = 5.6, |
| | | 12.4, 17.2 Hz, 6H), 2.23 (br s, 1H), 2.08 (td, J = 4.4, 8.4 Hz, 1H), 1.84 (br dd, J = 4.0, |
| | | 12.4 Hz, 1H), 1.82-1.66 (m, 2H) |
| I-140 | 914.6 | 11.10 (s, 1H), 8.14 (s, 1H), 8.12 (s, 1H), 7.70 (d, J = 2.4 Hz, 1H), 7.40-7.34 (m, 1H), |
| | | 7.31-7.22 (m, 1H), 7.05-6.97 (m, 3H), 6.90 (br d, J = 16.0 Hz, 1H), 6.27 (br d, J = |
| | | 14.0 Hz, 1H), 5.38 (br dd, J = 5.6, 12.2 Hz, 1H), 4.64 (t, J = 6.8 Hz, 2H), 4.40-4.28 |
| | | (m, 3H), 4.00-3.83 (m, 4H), 3.65-3.49 (m, 7H), 3.20-3.07 (m, 4H), 3.04 (br s, |
| | | 3H), 2.95-2.79 (m, 5H), 2.78-2.55 (m, 6H), 2.45 (br s, 2H), 2.33 (br d, J = 1.6 Hz, |
| | | 2H), 2.25 (br s, 2H), 2.03-1.95 (m, 1H), 1.85-1.70 (m, 8H), 1.25 (br s, 1H) |
| I-142 | 792.7 | 11.74-11.66 (m, 1H), 10.79 (s, 1H), 8.12 (s, 1H), 7.70 (br s, 1H), 7.45-7.36 (m, |
| | | 1H), 7.24-7.18 (m, 1H), 6.92 (br t, J = 8.8 Hz, 1H), 6.85 (br s, 1H), 6.55 (br d, J = |
| | | 14.0 Hz, 1H), 6.41 (br d, J = 9.2 Hz, 1H), 6.28 (br s, 1H), 5.95-5.76 (m, 1H), 4.63 |
| | | (br s, 2H), 4.37-4.31 (m, 2H), 4.25 (br d, J = 10.0 Hz, 2H), 3.90-3.81 (m, 1H), |
| | | 3.78-3.71 (m, 1H), 3.62-3.54 (m, 2H), 3.27 (br s, 6H), 3.17-3.07 (m, 4H), 2.80-2.64 |
| | | (m, 6H), 2.32 (br s, 2H), 2.12-2.06 (m, 1H), 1.91-1.79 (m, 3H), 1.62-1.46 (m, 2H) |
| I-335 | 870.4 | 11.92 (s, 1H), 11.11 (s, 1H), 8.43 (s, 1H), 8.10 (d, J = 10.8 Hz, 1H), 7.83 (dd, J = 2.0, |
| | | 8.7 Hz, 1H), 7.69 (d, J = 10.0 Hz, 1H), 7.44 (s, 1H), 7.39 (d, J = 8.4 Hz, 1H), 7.27 |
| | | (d, J = 8.4 Hz, 1H), 7.15 (d, J = 8.4 Hz, 1H), 7.04-6.97 (m, 1H), 6.94-6.72 (m, 2H), |
| | | 5.38 (dd, J = 5.2, 12.8 Hz, 1H), 4.61 (q, J = 6.8 Hz, 2H), 4.47 (t, J = 11.2 Hz, 1H), |
| | | 3.96-3.81 (m, 1H), 3.72-3.57 (m, 2H), 3.52 (s, 2H), 3.33 (s, 6H), 3.24-3.13 (m, |
| | | 2H), 3.12-2.98 (m, 4H), 2.97-2.81 (m, 2H), 2.81-2.58 (m, 4H), 2.08-1.92 (m, |
| | | 3H), 1.91-1.73 (m, 3H), 1.66-1.40 (m, 6H) |
| I-392 | 898.3 | 11.99-11.89 (m, 1H), 11.11 (s, 1H), 8.47-8.39 (m, 1H), 8.14-8.03 (m, 1H), 7.87- |
| | | 7.77 (m, 1H), 7.69 (d, J = 10.0 Hz, 1H), 7.46-7.40 (m, 1H), 7.38-7.32 (m, 1H), |
| | | 7.30-7.23 (m, 1H), 7.15 (d, J = 8.4 Hz, 1H), 7.04-6.94 (m, 1H), 6.92-6.85 (m, |
| | | 1H), 6.72-6.67 (m, 1H), 5.43-5.32 (m, 1H), 4.64-4.57 (m, 2H), 4.51-4.42 (m, |
| | | 1H), 4.34-4.25 (m, 2H), 4.20-4.10 (m, 1H), 3.88 (br s, 1H), 3.39 (s, 3H), 3.22- |
| | | 3.12 (m, 1H), 3.11-2.97 (m, 4H), 2.96-2.87 (m, 2H), 2.80-2.53 (m, 7H), 2.08- |
| | | 1.98 (m, 1H), 1.91-1.65 (m, 7H), 1.45 (br d. J = 12.0 Hz, 4H), 1.28-1.18 (m, 4H), |
| | | 1.13-1.01 (m, 2H) |
| I-513 | 820.3 | 11.06-10.82 (m, 1H), 7.80-7.67 (m, 3H), 7.61-7.33 (m, 6H), 7.11 (d, J = 8.8 Hz, |
| | | 2H), 6.29-6.07 (m, 2H), 4.45-4.22 (m, 5H), 3.88-3.67 (m, 2H), 3.66-3.56 (m, |
| | | 2H), 3.54-3.40 (m, 2H), 3.39-3.33 (m, 2H), 3.27 (s, 6H), 3.03 (s, 2H), 3.01-2.92 |
| | | (m, 2H), 2.85-2.74 (m, 1H), 2.57 (d, J = 4.0 Hz, 1H), 2.43-2.35 (m, 1H), 2.25 (s, |
| | | 2H), 2.07-1.97 (m, 1H) |
| I-573 | 820.2 | 12.14 (br d, J = 7.6 Hz, 1H), 10.54 (s, 1H), 7.71 (br s, 1H), 7.70-7.64 (m, 2H), |
| | | 7.58-7.54 (m, 2H), 7.51-7.47 (m, 1H), 7.45-7.36 (m, 1H), 7.09 (d, J = 8.8 Hz, 2H), |
| | | 7.01-6.93 (m, 1H), 6.88-6.83 (m, 1H), 6.23-6.17 (m, 1H), 6.17-6.12 (m, 1H), |
| | | 4.40-4.35 (m, 3H), 4.34 (br s, 1H), 3.86-3.73 (m, 3H), 3.69-3.48 (m, 6H), 3.27- |
| | | 3.06 (m, 8H), 3.03-2.92 (m, 3H), 2.79-2.74 (m, 2H), 2.36-2.25 (m, 2H) |
| I-574 | 833.3 | 12.18 (br d, J = 8.4 Hz, 1H), 10.51 (s, 1H), 7.74-7.69 (m, 1H), 7.64 (d, J = 8.4 Hz, |
| | | 1H), 7.59 (br d, J = 8.0 Hz, 2H), 7.56-7.54 (m, 1H), 7.41 (dd, J = 1.6, 11.6 Hz, 1H), |
| | | 7.39-7.36 (m, 1H), 7.31 (d, J = 8.0 Hz, 2H), 7.06-6.97 (m, 1H), 6.85 (br s, 1H), |
| | | 6.22-6.19 (m, 1H), 6.17-6.13 (m, 1H), 4.50-4.41 (m, 1H), 4.39-4.36 (m, 2H), |
| | | 4.36-4.29 (m, 2H), 3.79-3.72 (m, 1H), 3.66-3.57 (m, 4H), 3.22-3.05 (m, 6H), |
| | | 3.01-2.95 (m, 3H), 2.78-2.71 (m, 3H), 2.61 (br d, J = 7.2 Hz, 2H), 2.35-2.26 (m, |
| | | 2H), 1.92-1.83 (m, 1H), 1.76-1.57 (m, 2H), 1.26-1.17 (m, 2H) |
| I-576 | 860.1 | 12.13-12.08 (m, 1H), 10.52 (s, 1H), 7.81 (s, 1H), 7.71 (dd, J = 2.4, 11.2 Hz, 1H), |
| | | 7.67 (s, 2H), 7.51 (br d, J = 8.4 Hz, 2H), 7.44-7.36 (m, 1H), 7.07 (br d, J = 8.8 Hz, |
| | | 2H), 6.98-6.91 (m, 1H), 6.84 (br s, 1H), 6.21-6.17 (m, 1H), 6.13 (br s, 1H), 4.39- |
| | | 4.27 (m, 5H), 4.13 (s, 2H), 3.84 (s, 2H), 3.80-3.74 (m, 1H), 3.68-3.52 (m, 4H), |
| | | 3.25-3.17 (m, 6H), 3.12-3.07 (m, 2H), 2.99-2.95 (m, 2H), 2.78-2.73 (m, 2H), |
| | | 2.38-2.29 (m, 2H), 1.86 (br s, 4H) |
| I-595 | 819.3 | 12.19 (br d, J = 8.8 Hz, 1H), 10.52 (s, 1H), 7.71 (dd, J = 2.0, 11.2 Hz, 1H), 7.67 (d, |
| | | J = 8.4 Hz, 1H), 7.64-7.61 (m, 2H), 7.60 (s, 1H), 7.48 (dd, J = 2.0, 8.4 Hz, 1H), |
| | | 7.43-7.37 (m, 3H), 7.06-6.98 (m, 1H), 6.86 (br s, 1H), 6.21-6.13 (m, 2H), 4.74-4.58 |
| | | (m, 1H), 4.39-4.35 (m, 3H), 4.34-4.29 (m, 1H), 3.82-3.70 (m, 2H), 3.69-3.56 |
| | | (m, 4H), 3.21-3.04 (m, 6H), 3.00-2.96 (m, 2H), 2.91 (br dd, J = 10.8, 14.9 Hz, 2H), |
| | | 2.77-2.73 (m, 2H), 2.33 (br d, J = 1.6 Hz, 2H), 1.98-1.78 (m, |
| | | 2H), 1.75-1.66 (m, 2H) |
| I-736 | 902.4 | 12.21-12.13 (m, 1H), 10.51 (s, 1H), 7.74-7.69 (m, 1H), 7.65-7.65 (m, 1H), 7.64 |
| | | (d, J = 8.4 Hz, 1H), 7.61-7.57 (m, 3H), 7.45-7.38 (m, 2H), 7.36 (br d, J = 8.0 Hz, |
| | | 2H), 7.06-6.97 (m, 1H), 6.85 (br s, 1H), 6.20 (t, J = 1.6 Hz, 1H), 6.18-6.13 (m, |
| | | 1H), 4.55-4.44 (m, 1H), 4.44-4.32 (m, 4H), 4.30 (br s, 1H), 3.81-3.74 (m, 1H), |
| | | 3.67-3.54 (m, 5H), 3.06-2.94 (m, 7H), 2.86-2.65 (m, 4H), 2.61-2.53 (m, 2H), |
| | | 2.39-2.22 (m, 5H), 1.88-1.78 (m, 3H), 1.74-1.59 (m, 3H), 1.54-1.41 (m, 2H) |
| I-1182 | 874.4 | 12.19 (br d, J = 8.8 Hz, 1H), 10.53 (s, 1H), 7.81 (s, 1H), 7.76-7.63 (m, 3H), 7.60 |
| | | (br d, J = 8.0 Hz, 2H), 7.46-7.33 (m, 3H), 7.08-6.97 (m, 1H), 6.86 (br s, 1H), |
| | | 6.27-6.10 (m, 2H), 4.41-4.28 (m, 5H), 4.26-4.07 (m, 2H), 3.97-3.86 (m, 1H), |
| | | 3.83-3.74 (m, 1H), 3.68-3.56 (m, 3H), 3.24-3.14 (m, 4H), 3.06-2.88 (m, 6H), |
| | | 2.82-2.72 (m, 2H), 2.58 (br d, J = 12.0 Hz, 2H), 2.38-2.24 (m, 2H), 1.95 (br t, J = |
| | | 10.8 Hz, 2H), 1.84 (br s, 2H), 1.78-1.67 (m, 2H). |
| I-1183 | 902.4 | 12.11 (br d, J = 8.0 Hz, 1H), 10.52 (s, 1H), 7.78-7.67 (m, 1H), 7.65 (d, J = 8.0 Hz, |
| | | 1H), 7.57 (d, J = 2.0 Hz, 1H), 7.53-7.47 (m, 2H), 7.45-7.35 (m, 2H), 7.08-7.00 |
| | | (m, 2H), 6.99-6.91 (m, 1H), 6.86 (br s, 1H), 6.24-6.08 (m, 2H), 4.61-4.42 (m, |
| | | 1H), 4.40-4.28 (m, 4H), 3.86-3.73 (m, 3H), 3.69-3.54 (m, 4H), 3.24-3.02 (m, |
| | | 6H), 3.02-2.89 (m, 3H), 2.78-2.73 (m, 2H), 2.73-2.57 (m, 3H), 2.36-2.24 (m, |
| | | 2H), 1.87-1.62 (m, 4H), 1.33 (br s, 2H), 1.30 (br s, 2H), 1.23 (br d, J = 3.2 Hz, |
| | | 1H), 1.18-1.11 (m, 1H) |
| I-1184 | 903.4 | 12.10 (br d, J = 8.0 Hz, 1H), 10.51 (s, 1H), 7.71 (dd, J = 2.0, 11.2 Hz, 1H), 7.65 (d, |
| | | J = 8.4 Hz, 1H), 7.57 (d, J = 2.0 Hz, 1H), 7.53-7.47 (m, 2H), 7.46-7.34 (m, 2H), |
| | | 7.05 (br d, J = 8.8 Hz, 2H), 6.99-6.91 (m, 1H), 6.85 (br s, 1H), 6.23-6.11 (m, |
| | | 2H), 4.40-4.28 (m, 4H), 3.84-3.73 (m, 3H), 3.68-3.56 (m, 5H), 3.18-3.06 (m, |
| | | 4H), 3.03-2.92 (m, 3H), 2.80-2.65 (m, 5H), 2.54 (s, 6H), 2.43-2.21 (m, 3H), |
| | | 1.86 (br d, J = 10.0 Hz, 2H), 1.61-1.48 (m, 2H) |
| I-1186 | 939.4 | 12.31-12.10 (m, 1H), 10.68-10.32 (m, 1H), 7.73-7.69 (m, 1H), 7.65 (d, J = 8.4 |
| | | Hz, 1H), 7.58 (d, J = 1.6 Hz, 1H), 7.44-7.35 (m, 2H), 7.30-7.23 (m, 1H), 7.03- |
| | | 6.93 (m, 2H), 6.64 ( s, 1H), 6.22-6.16 (m, 1H), 6.13 ( d, J = 4.0 Hz, 1H), 4.36 ( d, |
| | | J = 6.8 Hz, 3H), 4.28 (s, 1H), 3.83-3.72 (m, 1H), 3.67-3.60 (m, 3H), 3.57 (t, J = |
| | | 5.2 Hz, 2H), 3.55-3.50 (m, 2H), 3.20-3.13 (m, 6H), 3.01-2.94 (m, 4H), 2.83- |
| | | 2.77 (m, 2H), 2.75 (d, J = 4.8 Hz, 2H), 2.67-2.59 (m, 2H), 2.33 (s, 2H), 2.29-2.23 |
| | | (m, 2H), 1.88 (d, J = 10.4 Hz, 2H), 1.65-1.57 (m, 2H) |
| I-1187 | 953.4 | 12.29-12.22 (m, 1H), 10.50 (s, 1H), 7.74-7.68 (m, 1H), 7.64 (d, J = 8.4 Hz, 1H), |
| | | 7.56 (d, J = 1.6 Hz, 1H), 7.42 (dd, J = 1.6, 8.4 Hz, 1H), 7.39-7.34 (m, 2H), 7.32- |
| | | 7.28 (m, 1H), 7.10-7.02 (m, 1H), 6.67-6.63 (m, 1H), 6.20 (s, 2H), 4.39-4.34 (m, |
| | | 3H), 4.29 (br d, J = 0.8 Hz, 1H), 3.75 (br dd, J = 6.8, 12.8 Hz, 1H), 3.65-3.55 (m, |
| | | 8H), 3.19-3.04 (m, 7H), 3.01-2.89 (m, 6H), 2.77-2.71 (m, 2H), 2.36-2.22 (m, |
| | | 4H), 2.06-1.99 (m, 2H), 1.75-1.69 (m, 2H), 1.49-1.40 (m, 2H) |
| I-1188 | 953.4 | 12.37-11.99 (m, 1H), 10.64-10.41 (m, 1H), 7.74-7.68 (m, 1H), 7.65 (d, J = 8.0 |
| | | Hz, 1H), 7.58 (d, J = 2.0 Hz, 1H), 7.44-7.35 (m, 2H), 7.32-7.23 (m, 1H), 7.04- |
| | | 6.91 (m, 2H), 6.67-6.60 (m, 1H), 6.24-6.06 (m, 2H), 4.39-4.27 (m, 4H), 4.09- |
| | | 4.08 (m, 1H), 3.79-3.72 (m, 1H), 3.66-3.55 (m, 5H), 3.50-3.42 (m, 3H), 3.19- |
| | | 3.02 (m, 6H), 3.01-2.92 (m, 3H), 2.79-2.71 (m, 4H), 2.44-2.36 (m, 4H), 2.33 (s, |
| | | 2H), 2.24 (d, J = 6.4 Hz, 2H), 1.87-1.80 (m, 2H), 1.76-1.64 (m, 1H), 1.36-1.24 |
| | | (m, 2H) |
| I-1189 | 917.3 | 12.11 (br d, J = 8.0 Hz, 1H), 10.51 (s, 1H), 7.71 (dd, J = 1.6, 11.2 Hz, 1H), 7.64 (d, |
| | | J = 8.0 Hz, 1H), 7.57-7.49 (m, 3H), 7.46-7.34 (m, 2H), 7.04 (br d, J = 8.8 Hz, |
| | | 2H), 6.95 (br dd, J = 6.0, 18.4 Hz, 1H), 6.85 (br s, 1H), 6.23-6.10 (m, 2H), 4.50- |
| | | 4.29 (m, 5H), 3.78-3.72 (m, 1H), 3.68-3.54 (m, 5H), 3.22-2.91 (m, 16H), 2.81- |
| | | 2.70 (m, 3H), 2.34-2.19 (m, 4H), 1.90-1.65 (m, 3H), 1.18-1.06 (m, 2H). |
| I-1190 | 917.5 | 12.22-11.97 (m, 1H), 10.78-10.56 (m, 1H), 8.21-8.11 (m, 1H), 7.75-7.68 (m, |
| | | 1H), 7.56 (s, 1H), 7.46-7.37 (m, 1H), 7.32-7.25 (m, 1H), 7.23-7.13 (m, 2H), |
| | | 7.02-6.96 (m, 1H), 6.94-6.87 (m, 1H), 6.53-6.44 (m, 1H), 6.24 (s, 1H), 4.55- |
| | | 4.43 (m, 1H), 4.41-4.31 (m, 2H), 3.93-3.84 (m, 1H), 3.81 (brt, J = 6.6 Hz, 2H), |
| | | 3.15 (br s, 8H), 3.10-3.02 (m, 4H), 2.96-2.90 (m, 2H), 2.89-2.76 (m, 5H), 2.74- |
| | | 2.62 (m, 5H), 1.98-1.82 (m, 2H), 1.80-1.72 (m, 1H), 1.55-1.38 (m, 1H). |
| I-1191 | 943.3 | 12.11 (br d, J = 6.4 Hz, 1H), 10.51 (br s, 1H), 7.75-7.68 (m, 1H), 7.64 (d, J = 8.4 |
| | | Hz, 1H), 7.57-7.49 (m, 3H), 7.46-7.34 (m, 2H), 7.04 (br d, J = 8.4 Hz, 2H), |
| | | 6.98-6.90 (m, 1H), 6.84 (br s, 1H), 6.22-6.10 (m, 2H), 4.39-4.28 (m, 4H), 3.80-3.71 |
| | | (m, 1H), 3.68-3.48 (m, 5H), 3.25-3.06 (m, 10H), 3.03-2.92 (m, 3H), 2.78-2.69 |
| | | (m, 3H), 2.45-2.18 (m, 7H), 2.02 (br d, J = 1.6 Hz, 2H), 1.66-1.45 (m, 6H) |
| I-1192 | 943.9 | 12.10 (br d, J = 7.2 Hz, 1H), 10.51 (s, 1H), 7.74-7.68 (m, 1H), 7.64 (d, J = 8.4 Hz, |
| | | 1H), 7.57-7.46 (m, 3H), 7.46-7.35 (m, 2H), 7.04 (br d, J = 8.4 Hz, 2H), 6.94 (br |
| | | dd, J = 6.4, 18.0 Hz, 1H), 6.84 (br s, 1H), 6.28-6.07 (m, 2H), 4.40-4.26 (m, 4H), |
| | | 3.77 (br s, 6H), 3.60-3.54 (m, 3H), 3.12 (br s, 8H), 3.02 (br s, 4H), 2.86-2.69 (m, |
| | | 5H), 2.37-2.21 (m, 3H), 1.81-1.63 (m, 6H), 1.37-1.21 (m, 2H) |
| I-1193 | 971.4 | 12.28-11.94 (m, 1H), 10.51 (s, 1H), 7.74-7.36 (m, 7H), 7.08-6.84 (m, 4H), |
| | | 6.45-5.95 (m, 2H), 4.40-4.34 (m, 3H), 4.34-4.25 (m, 1H), 3.85-3.72 (m, 3H), 3.69- |
| | | 3.42 (m, 6H), 3.27-2.89 (m, 9H), 2.83-2.55 (m, 6H), 2.45-2.21 (m, |
| | | 4H), 1.91-1.79 (m, 2H), 1.69-1.32 (m, 11H). |
| I-1194 | 972.4 | 12.12 (d, J = 8.4 Hz, 1H), 10.63-10.37 (m, 1H), 7.76-7.68 (m, 1H), 7.67-7.60 |
| | | (m, 1H), 7.58-7.49 (m, 3H), 7.45-7.33 (m, 2H), 7.04 (d, J = 8.0 Hz, 2H), 6.99- |
| | | 6.90 (m, 1H), 6.88-6.81 (m, 1H), 6.27-6.01 (m, 2H), 4.40-4.34 (m, 3H), 4.30 (s, |
| | | 1H), 3.83-3.67 (m, 2H), 3.67-3.55 (m, 6H), 3.19 (s, 6H), 3.11 (s, 3H), 3.01-2.92 |
| | | (m, 3H), 2.78-2.73 (m, 2H), 2.67 (s, 4H), 2.35-2.25 (m, 3H), 1.82-1.62 (m, 4H), |
| | | 1.55-1.28 (m, 6H), 1.14 (d, J = 2.0 Hz, 2H). |
| I-1195 | 902.6 | 12.18 (dd, J = 1.2, 6.4 Hz, 1H), 10.52 (s, 1H), 7.74-7.68 (m, 1H), 7.65 (d, J = 8.4 |
| | | Hz, 1H), 7.62-7.57 (m, 3H), 7.45-7.34 (m, 4H), 7.05-6.96 (m, 1H), 6.86 (s, 1H), |
| | | 6.22-6.11 (m, 2H), 4.40-4.28 (m, 4H), 3.81-3.73 (m, 1H), 3.70-3.55 (m, 5H), |
| | | 3.38 (s, 2H), 3.23-3.04 (m, 6H), 3.02-2.93 (m, 3H), 2.80-2.65 (m, 4H), 2.38- |
| | | 2.22 (m, 5H), 2.02-1.85 (m, 4H), 1.62-1.51 (m, 4H) |
| I-1196 | 957.2 | 12.17-12.05 (m, 1H), 10.27 (s, 1H), 7.83-7.76 (m, 1H), 7.75-7.69 (m, 1H), 7.67- |
| | | 7.63 (m, 2H), 7.52 (d, J = 7.6 Hz, 2H), 7.44-7.37 (m, 1H), 7.10-7.02 (m, 2H), |
| | | 6.99-6.92 (m, 1H), 6.88-6.84 (m, 1H), 6.23-6.11 (m, 2H), 4.30 (s, 5H), 4.05- |
| | | 3.94 (m, 2H), 3.76-3.69 (m, 2H), 3.66-3.57 (m, 3H), 3.22-3.18 (m, 6H), 3.16- |
| | | 3.03 (m, 6H), 3.01-2.93 (m, 3H), 2.81-2.66 (m, 3H), 2.35-2.31 (m, 2H), 2.17- |
| | | 2.11 (m, 2H), 1.92-1.86 (m, 2H), 1.74-1.68 (m, 2H), 1.50-1.43 (m, 2H), 1.24 (s, |
| | | 1H), 0.97-0.84 (m, 2H) |
| I-1197 | 460.1 | 12.23-11.97 (m, 1H), 10.69 (s, 1H), 8.75 (s, 1H), 8.29 (s, 1H), 7.79 (s, 1H), 7.74- |
| | | 7.69 (m, 1H), 7.50 (d, J = 8.4 Hz, 2H), 7.45-7.35 (m, 1H), 7.04 (r d, J = 8.8 Hz, |
| | | 2H), 6.98-6.90 (m, 1H), 6.85 (s, 1H), 6.22-6.16 (m, 1H), 6.13 ( s, 1H), 4.39- |
| | | 4.34 (m, 3H), 4.29 (s, 1H), 3.82-3.73 (m, 4H), 3.70-3.62 (m, 3H), 3.58 ( t, J = 5.6 |
| | | Hz, 1H), 3.45 (s, 2H), 3.22-3.02 (m, 6H), 3.01-2.92 (m, 3H), 2.78-2.74 (m, 2H), |
| | | 2.72 (d, J = 6.4 Hz, 2H), 2.45 (s, 1H), 2.35 ( d, J = 16.4 Hz, 3H), 2.29-2.19 (m, |
| | | 3H), 1.87-1.78 (m, 2H), 1.76-1.66 (m, 1H), 1.27-1.20 (m, 2H) |
| I-1198 | 974.2 | 12.33-12.17 (m, 1H), 10.63-10.42 (m, 1H), 8.65-8.42 (m, 1H), 7.74-7.65 (m, |
| | | 2H), 7.64-7.55 (m, 1H), 7.44-7.24 (m, 4H), 7.10-7.00 (m, 1H), 6.67-6.62 (m, |
| | | 1H), 6.22-6.12 (m, 2H), 4.38-4.28 (m, 4H), 3.83-3.70 (m, 2H), 3.68-3.53 (m, |
| | | 4H), 3.23-3.05 (m, 8H), 3.00 (br s, 6H), 2.76-2.67 (m, 4H), 2.34-2.25 (m, 2H), |
| | | 1.89-1.64 (m, 6H) |
| I-1199 | 874.3 | 12.20 (d, J = 8.3 Hz, 1H), 10.52 (s, 1H), 7.82-7.68 (m, 2H), 7.67-7.57 (m, 4H), |
| | | 7.47-7.34 (m, 3H), 7.14-6.95 (m, 1H), 6.88 (s, 1H), 6.34-5.97 (m, 2H), 4.41- |
| | | 4.30 (m, 4H), 4.04 (s, 2H), 3.76 (s, 3H), 3.68-3.56 (m, 4H), 3.23-3.07 (m, 6H), |
| | | 3.02-2.93 (m, 3H), 2.79-2.69 (m, 2H), 2.40-2.24 (m, 6H), 1.76 (s, 4H) |
| I-1200 | 903.4 | 12.28-12.14 (m, 1H), 10.87-10.79 (m, 1H), 8.50-8.41 (m, 1H), 8.15-8.06 (m, |
| | | 1H), 7.74-7.64 (m, 1H), 7.65-7.59 (m, 2H), 7.46-7.40 (m, 2H), 7.09-6.97 (m, |
| | | 4H), 6.90-6.77 (m, 1H), 6.24-6.10 (m, 1H), 4.66-4.58 (m, 2H), 4.41-4.31 (m, |
| | | 2H), 3.84-3.77 (m, 1H), 3.68-3.63 (m, 1H), 3.62-3.58 (m, 1H), 3.54-3.45 (m, |
| | | 4H), 3.25-3.01 (m, 6H), 2.74 (s, 3H), 2.72-2.60 (m, 2H), 2.58-2.53 (m, 2H), |
| | | 2.43-2.31 (m, 2H), 2.31-2.24 (m, 1H), 2.17 (s, 1H), 2.06-1.78 (m, 6H), 1.17- |
| | | 1.12 (m, 3H) |
| I-1201 | 937.3 | 12.37-12.06 (m, 1H), 11.02-10.85 (m, 1H), 7.74-7.68 (m, 1H), 7.53 (d, J = 8.4 |
| | | Hz, 1H), 7.43-7.36 (m, 2H), 7.33 (dd, J = 1.8, 8.2 Hz, 1H), 7.30-7.23 (m, 1H), |
| | | 7.03-6.92 (m, 2H), 6.66-6.61 (m, 1H), 6.21-6.16 (m, 1H), 6.15-6.10 (m, 1H), |
| | | 4.56-4.45 (m, 1H), 4.43-4.30 (m, 4H), 4.30-4.23 (m, 2H), 3.66-3.55 (m, 3H), |
| | | 3.53-3.47 (m, 2H), 3.19-3.02 (m, 6H), 3.00-2.93 (m, 3H), 2.80-2.67 (m, 4H), |
| | | 2.56 (br d, J = 3.0 Hz, 1H), 2.36-2.30 (m, 2H), 2.28-2.23 (m, 1H), 2.04-1.97 (m, |
| | | 1H), 1.85-1.66 (m, 4H), 1.38 (br d, J = 10.2 Hz, 2H), 1.29-1.14 (m, 3H) |
| I-1202 | 889.1 | 12.82-11.46 (m, 1H), 10.77-10.27 (m, 1H), 7.78 (s, 1H), 7.71 (dd, J = 2.0, 11.6 |
| | | Hz, 1H), 7.65 (s, 2H), 7.57 (br d, J = 8.0 Hz, 2H), 7.44-7.31 (m, 3H), 7.07-6.93 |
| | | (m, 1H), 6.84 (d, J = 3.2 Hz, 1H), 6.25-6.08 (m, 2H), 4.47-4.23 (m, 5H), 4.04 (s, |
| | | 2H), 3.84-3.71 (m, 4H), 3.67-3.55 (m, 5H), 3.10-2.90 (m, 6H), 2.81-2.71 (m, |
| | | 5H), 2.36-2.23 (m, 4H), 1.78-1.70 (m, 4H), 1.29-1.09 (m, 1H) |
| I-1203 | 849.2 | 12.21 (br d, J = 7.6 Hz, 1H), 10.52 (s, 1H), 7.72 (dd, J = 2.0, 11.2 Hz, 1H), 7.65 (d, |
| | | J = 8.0 Hz, 3H), 7.60 (d, J = 2.0 Hz, 1H), 7.47 (d, J = 8.0 Hz, 2H), 7.45-7.36 (m, |
| | | 2H), 7.10-6.98 (m, 1H), 6.87 (br s, 1H), 6.34-6.02 (m, 2H), 4.62 (s, 2H), 4.46- |
| | | 4.23 (m, 4H), 4.07-3.88 (m, 1H), 3.84-3.72 (m, 2H), 3.69-3.48 (m, 4H), 3.31- |
| | | 3.01 (m, 8H), 3.01-2.91 (m, 2H), 2.80-2.71 (m, 2H), 2.40-2.22 (m, 2H), 2.05- |
| | | 1.83 (m, 2H), 1.60 (br s, 2H) |
| I-1204 | 974.2 | 12.29-12.20 (m, 1H), 10.53 (s, 1H), 7.75-7.66 (m, 2H), 7.60 (br s, 1H), 7.47- |
| | | 7.30 (m, 4H), 7.10-6.99 (m, 1H), 6.63 (br d, J = 2.4 Hz, 1H), 6.23-6.11 (m, 2H), |
| | | 4.72-4.46 (m, 1H), 4.39-4.28 (m, 4H), 3.85-3.70 (m, 2H), 3.69-3.54 (m, 4H), |
| | | 3.22-3.02 (m, 9H), 3.00-2.93 (m, 3H), 2.89-2.81 (m, 1H), 2.78-2.66 (m, 4H), |
| | | 2.34-2.25 (m, 2H), 1.99-1.69 (m, 6H) |
| I-1205 | 974.5 | 12.35-12.18 (m, 1H), 10.52 (s, 1H), 7.75-7.68 (m, 1H), 7.67-7.62 (m, 1H), 7.59 |
| | | (d, J = 1.6 Hz, 1H), 7.50-7.36 (m, 4H), 7.14-7.03 (m, 1H), 6.64 (br d, J = 2.0 Hz, |
| | | 1H), 6.18 (br d, J = 14.9 Hz, 2H), 4.58-4.44 (m, 1H), 4.40-4.28 (m, 4H), 3.82- |
| | | 3.72 (m, 1H), 3.68-3.55 (m, 4H), 3.52-3.41 (m, 1H), 3.23-3.13 (m, 3H), 3.12- |
| | | 2.92 (m, 8H), 2.79-2.71 (m, 4H), 2.69-2.62 (m, 1H), 2.40-2.20 (m, 3H), 2.19- |
| | | 2.08 (m, 1H), 1.92-1.81 (m, 2H), 1.78-1.64 (m, 1H), 1.56-1.43 (m, 2H) |
| I-1206 | 938.3 | 12.33-12.06 (m, 1H), 10.96 (s, 1H), 7.80-7.61 (m, 1H), 7.56 (d, J = 8.1 Hz, 1H), |
| | | 7.46-7.32 (m, 3H), 7.31-7.24 (m, 1H), 7.08-6.88 (m, 2H), 6.71-6.56 (m, 1H), |
| | | 6.25-6.07 (m, 2H), 4.57-4.40 (m, 1H), 4.40-4.34 (m, 3H), 4.33-4.25 (m, 2H), |
| | | 3.67-3.57 (m, 3H), 3.20-3.18 (m, 1H), 3.12 (s, 6H), 3.08 (s, 2H), 3.01-2.94 (m, |
| | | 3H), 2.85-2.75 (m, 2H), 2.72 (s, 4H), 2.61-2.54 (m, 2H), 2.39-2.23 (m, 4H), |
| | | 2.05-1.98 (m, 1H), 1.95-1.73 (m, 2H), 1.44 (d, J = 10.0 Hz, 2H) |
| I-1207 | 974.4 | 12.47-12.06 (m, 1H), 10.53 (br s, 1H), 7.78-7.53 (m, 3H), 7.51-7.27 (m, 4H), |
| | | 7.19-7.03 (m, 1H), 6.67 (br d, J = 2.4 Hz, 1H), 6.29-6.07 (m, 2H), 4.60-4.47 (m, |
| | | 1H), 4.43-4.27 (m, 4H), 3.85-3.73 (m, 1H), 3.71-3.46 (m, 5H), 3.29-2.86 (m, |
| | | 12H), 2.84-2.65 (m, 5H), 2.39-2.24 (m, 2H), 2.21-2.07 (m, 1H), 1.96-1.81 (m, |
| | | 2H), 1.79-1.66 (m, 1H), 1.62-1.41 (m, 2H) |
| I-1208 | 974.4 | 12.44-12.18 (m, 1H), 10.63-10.41 (m, 1H), 7.79-7.53 (m, 3H), 7.50-7.30 (m, |
| | | 4H), 7.16-7.02 (m, 1H), 6.67 (br d, J = 2.4 Hz, 1H), 6.26-6.01 (m, 2H), 4.66- |
| | | 4.42 (m, 1H), 4.40-4.29 (m, 4H), 3.83-3.73 (m, 1H), 3.71-3.48 (m, 5H), 3.24- |
| | | 2.92 (m, 11H), 2.82-2.65 (m, 5H), 2.47-2.20 (m, 3H), 2.19-2.07 (m, 1H), 1.95- |
| | | 1.81 (m, 2H), 1.78-1.67 (m, 1H), 1.57-1.43 (m, 2H) |
| I-1209 | 974.5 | 12.41-12.10 (m, 1H), 10.63-10.43 (m, 1H), 7.71 (dd, J = 2.1, 11.6 Hz, 1H), 7.65 |
| | | (d, J = 8.3 Hz, 1H), 7.59 (d, J = 1.9 Hz, 1H), 7.50-7.36 (m, 4H), 7.13-7.04 (m, |
| | | 1H), 6.67-6.60 (m, 1H), 6.22-6.13 (m, 2H), 4.57-4.45 (m, 1H), 4.39-4.34 (m, |
| | | 3H), 4.33 (br s, 1H), 3.81-3.73 (m, 1H), 3.66-3.55 (m, 4H), 3.53-3.43 (m, 1H), |
| | | 3.24-3.15 (m, 3H), 3.12-2.94 (m, 8H), 2.79-2.72 (m, 4H), 2.69-2.63 (m, 1H), |
| | | 2.42-2.20 (m, 3H), 2.19-2.10 (m, 1H), 1.86 (br d, J = 10.9 Hz, 2H), 1.78-1.66 |
| | | (m, 1H), 1.56-1.42 (m, 2H) |
| I-1210 | 975.1 | 12.53-11.97 (m, 1H), 10.74-10.34 (m, 1H), 8.16-8.05 (m, 1H), 7.79-7.51 (m, |
| | | 3H), 7.48-7.33 (m, 3H), 7.18-7.04 (m, 1H), 6.70-6.63 (m, 1H), 6.23-6.13 (m, |
| | | 1H), 4.72-4.47 (m, 3H), 4.44-4.29 (m, 2H), 3.85-3.73 (m, 1H), 3.70-3.47 (m, |
| | | 5H), 3.30-2.97 (m, 12H), 2.83-2.67 (m, 5H), 2.39-2.25 (m, 2H), 2.21-2.08 (m, |
| | | 1H), 1.95-1.82 (m, 2H), 1.78-1.66 (m, 1H), 1.59-1.42 (m, 2H) |
| I-1211 | 975.5 | 10.59 (s, 1H), 8.17 (d, J = 12.4 Hz, 1H), 7.79-7.63 (m, 3H), 7.55-7.38 (m, 3H), |
| | | 7.22-7.10 (m, 1H), 6.73 (br s, 1H), 6.24 (br dd, J = 4.4, 6.0 Hz, 1H), 4.73-4.64 |
| | | (m, 2H), 4.63-4.52 (m, 1H), 4.48-4.36 (m, 2H), 3.83 (br s, 1H), 3.74-3.64 (m, |
| | | 4H), 3.62-3.52 (m, 1H), 3.33-3.01 (m, 12H), 2.87-2.72 (m, 5H), 2.44-2.30 (m, |
| | | 2H), 2.28-2.12 (m, 1H), 2.03-1.86 (m, 2H), 1.84-1.71 (m, 1H), |
| | | 1.66-1.45 (m, 2H) |
| I-1185 | 791.4 | 12.08 (s, 1H), 10.84-10.74 (m, 1H), 8.14-8.06 (m, 1H), 7.69 (d, J = 10.4 Hz, 1H), |
| | | 7.59 (d, J = 7.6 Hz, 2H), 7.40 (d, J = 8.0 Hz, 2H), 7.10-7.01 (m, 2H), 6.89-6.81 |
| | | (m, 1H), 6.51-6.41 (m, 2H), 6.00 (d, J = 7.6 Hz, 1H), 4.61 (q, J = 6.4 Hz, 2H), |
| | | 4.52-4.44 (m, 1H), 4.37-4.26 (m, 1H), 3.95-3.87 (m, 1H), 3.24-3.14 (m, 3H), |
| | | 3.11-3.00 (m, 6H), 2.84-2.69 (m, 3H), 2.68-2.55 (m, 3H), 2.14-2.06 (m, 1H), |
| | | 1.98-1.84 (m, 7H), 1.81-1.75 (m, 1H), 1.71-1.60 (m, 4H), 1.58-1.41 (m, 1H). |
| I-1212 | 951.4 | 12.41-11.84 (m, 1H), 10.98-10.78 (m, 1H), 7.71 (dd, J = 1.9, 11.6 Hz, 1H), 7.45- |
| | | 7.35 (m, 1H), 7.32 (s, 1H), 7.29-7.24 (m, 1H), 7.19 (d, J = 1.3 Hz, 1H), 7.02 (br d, |
| | | J = 6.1 Hz, 2H), 6.64 (br d, J = 1.9 Hz, 1H), 6.21-6.16 (m, 1H), 6.15-6.10 (m, |
| | | 1H), 4.59-4.44 (m, 1H), 4.40-4.26 (m, 6H), 3.66-3.56 (m, 3H), 3.51 (br d, J = |
| | | 10.4 Hz, 2H), 3.15-3.02 (m, 5H), 3.00-2.93 (m, 3H), 2.76-2.65 (m, 4H), 2.39 (s, |
| | | 3H), 2.32 (br d, J = 1.9 Hz, 2H), 2.27 (br dd, J = 3.4, 9.0 Hz, 1H), 2.03-1.97 (m, |
| | | 1H), 1.84-1.65 (m, 4H), 1.45-1.35 (m, 3H), 1.33-1.12 (m, 4H) |
| I-1318 | 856.2 | 11.98 (s, 1H), 11.10 (s, 1H), 7.18 (d, J = 8.4 Hz, 1H), 7.03-6.92 (m, 3H), 6.87 (d, |
| | | J = 3.6 Hz, 1H), 6.68 (s, 1H), 6.60 (d, J = 8.8 Hz, 1H), 6.50 (s, 1H), 6.14 (s, 1H), 5.38 |
| | | (dd, J = 5.2, 12.4 Hz, 1H), 4.79-4.46 (m, 1H), 4.44-4.29 (m, 1H), 3.99-3.84 (m, |
| | | 1H), 3.72 (s, 3H), 3.62 (s, 3H), 3.35-3.33 (m, 2H), 3.27 (s, 4H), 3.22-2.96 (m, |
| | | 8H), 2.95-2.85 (m, 1H), 2.77-2.57 (m, 9H), 2.05-1.96 (m, 1H), 1.63 (s, 2H), |
| | | 1.54 (s, 2H) |
| I-1316 | 872.3 | 11.96 (s, 1H), 11.09 (s, 1H), 8.06 (s, 1H), 7.75-7.71 (m, 1H), 7.21-7.15 (m, 1H), |
| | | 6.99 (d, J = 5.6 Hz, 2H), 6.96-6.93 (m, 1H), 6.84 (d, J = 6.4 Hz, 1H), 6.67 (s, 1H), |
| | | 6.60 (t, J = 6.8 Hz, 1H), 6.49 (br s, 1H), 6.19-6.12 (m, 1H), 5.62-5.56 (m, 2H), |
| | | 5.39-5.35 (m, 1H), 4.46-4.34 (m, 2H), 3.74-3.70 (m, 4H), 3.62 (s, 3H), 3.30- |
| | | 3.22 (m, 5H), 3.20-3.03 (m, 7H), 2.95-2.84 (m, 1H), 2.77-2.64 (m, 8H), 2.60 (br |
| | | s, 1H), 2.48-2.46 (m, 1H), 2.36-2.29 (m, 1H), 2.04-1.96 (m, 1H) |
| I-1313 | 886.3 | 11.96 (s, 1H), 11.10 (s, 1H), 8.46-8.37 (m, 1H), 7.21-7.15 (m, 1H), 7.02-6.92 |
| | | (m, 3H), 6.91-6.80 (m, 1H), 6.67 (s, 1H), 6.60 (d, J = 8.8 Hz, 1H), 6.48 (t, J = 2.4 |
| | | Hz, 1H), 6.31-6.23 (m, 1H), 6.15-6.07 (m, 1H), 5.38 (dd, J = 5.2, 12.4 Hz, 1H), |
| | | 4.36 (s, 2H), 3.71 (s, 3H), 3.68-3.60 (m, 5H), 3.26 (s, 6H), 3.18-3.10 (m, 4H), |
| | | 3.07-2.97 (m, 4H), 2.92-2.81 (m, 3H), 2.67 (s, 8H), 2.41-2.25 (m, 2H), 2.05- |
| | | 1.95 (m, 1H) |
| I-1295 | 896.3 | 11.95 (d, J = 6.4 Hz, 1H), 11.10 (s, 1H), 8.51-8.39 (m, 1H), 7.67 (m, 1H), 7.35- |
| | | 7.25 (m, 1H), 7.22-7.12 (m, 2H), 7.03-6.91 (m, 3H), 6.90-6.80 (m, 1H), 6.67 (s, |
| | | 1H), 6.60 (dd, J = 1.6, 8.4 Hz, 1H), 6.48 (s, 1H), 6.14-6.06 (m, 1H), 5.38 (dd, J = |
| | | 5.6, 12.4 Hz, 1H), 4.35 (s, 2H), 3.75-3.68 (m, 3H), 3.67-3.59 (m, 5H), 3.26 (s, |
| | | 4H), 3.21-2.95 (m, 10H), 2.94-2.79 (m, 3H), 2.78-2.59 (m, 8H), 2.35 (d, J = 2.0 |
| | | Hz, 1H), 2.25 (s, 1H), 2.05-1.96 (m, 1H) |
| I-1315 | 897.7 | 11.70-11.46 (m, 1H), 10.94-10.71 (m, 1H), 7.57-7.21 (m, 5H), 7.17 (d, J = 8.0 |
| | | Hz, 1H), 7.05-6.92 (m, 3H), 6.68 (d, J = 2.0 Hz, 1H), 6.63 (d, J = 8.4 Hz, 1H), |
| | | 6.49 (d, J = 2.8 Hz, 1H), 6.03 (s, 1H), 5.46 (s, 2H), 5.38-5.26 (m, 1H), 4.49-4.14 |
| | | (m, 2H), 3.72 (s, 3H), 3.64 (s, 3H), 3.34-3.24 (m, 4H), 3.17 (d, J = 7.8 Hz, 5H), |
| | | 3.12 (s, 6H), 2.97-2.84 (m, 2H), 2.75-2.62 (m, 8H), 2.27-2.12 (m, |
| | | 1H), 2.08-2.00 (m, 1H) |
| I-1319 | 912.4 | 12.05-11.83 (m, 1H), 11.11 (s, 1H), 7.57 (dd, J = 1.6, 6.8 Hz, 1H), 7.48-7.31 (m, |
| | | 1H), 7.24-7.12 (m, 1H), 7.05-6.87 (m, 3H), 6.85-6.75 (m, 1H), 6.67 (s, 1H), |
| | | 6.64-6.56 (m, 1H), 6.48 (t, J = 2.4 Hz, 1H), 6.45-6.24 (m, 2H), 6.12-6.03 (m, |
| | | 1H), 6.01-5.83 (m, 1H), 5.38 (dd, J = 5.2, 12.4 Hz, 1H), 4.57-4.16 (m, 2H), 3.73- |
| | | 3.69 (m, 3H), 3.66-3.62 (m, 2H), 3.51-3.30 (m, 6H), 3.26-3.22 (m, 3H), 3.20- |
| | | 3.05 (m, 6H), 2.96-2.86 (m, 1H), 2.78-2.58 (m, 8H), 2.38-2.07 (m, |
| | | 2H), 2.05-1.95 (m, 1H), 1.53-1.31 (m, 3H) |
| I-1317 | 920.3 | 11.98-11.90 (m, 1H), 11.10 (s, 1H), 7.81-7.69 (m, 1H), 7.68-7.59 (m, 2H), 7.52- |
| | | 7.42 (m, 1H), 7.17 (dd, J = 2.4, 8.4 Hz, 1H), 6.99 (d, J = 5.4 Hz, 2H), 6.97-6.93 |
| | | (m, 1H), 6.89-6.80 (m, 1H), 6.67 (s, 1H), 6.60 (d, J = 8.4 Hz, 1H), 6.50-6.47 (m, |
| | | 1H), 6.14-6.06 (m, 1H), 5.44-5.33 (m, 1H), 4.39-4.29 (m, 2H), 3.73-3.68 (m, |
| | | 3H), 3.66-3.63 (m, 1H), 3.62 (s, 4H), 3.37 (d, J = 4.0 Hz, 2H), 3.26 (s, 4H), 3.20 |
| | | (d, J = 4.0 Hz, 1H), 3.16-3.11 (m, 3H), 3.08-3.01 (m, 2H), 2.87 (s, 3H), 2.80- |
| | | 2.75 (m, 2H), 2.67 (d, J = 1.6 Hz, 8H), 2.34-2.32 (m, 1H), 2.29- |
| | | 2.23 (m, 1H), 2.04-1.96 (m, 1H) |
| I-1306 | 901.7 | 11.99-11.91 (m, 1H), 11.10 (s, 1H), 7.33-7.23 (m, 1H), 7.18 (d, J = 8.4 Hz, 1H), |
| | | 7.02-6.96 (m, 2H), 6.96-6.90 (m, 2H), 6.90-6.82 (m, 2H), 6.67 (s, 1H), 6.60 (d, |
| | | J = 8.4 Hz, 1H), 6.51-6.46 (d, J = 2.0 Hz, 1H), 6.13-6.06 (m, 1H), 5.38 (dd, J = |
| | | 5.2, 12.4 Hz, 1H), 4.41-4.27 (m, 2H), 3.74-3.69 (m, 3H), 3.68-3.64 (m, 1H), |
| | | 3.62 (s, 3H), 3.60-3.58 (m, 1H), 3.28-3.24 (m, 4H), 3.18-3.10 (m, 4H), 3.10- |
| | | 3.01 (m, 5H), 2.96-2.84 (m, 2H), 2.81-2.71 (m, 3H), 2.71-2.60 (m, 7H), 2.36- |
| | | 2.30 (m, 1H), 2.29-2.23 (m, 1H), 2.04-1.93 (m, 1H) |
| I-1311 | 886.6 | 11.99-11.92 (m, 1H), 11.10 (s, 1H), 8.81-8.72 (m, 1H), 7.18 (d, J = 8.4 Hz, 1H), |
| | | 7.02-6.92 (m, 3H), 6.91-6.82 (m, 1H), 6.67 (s, 1H), 6.60 (dd, J = 1.6, 8.4 Hz, |
| | | 1H), 6.55-6.46 (m, 2H), 6.15-6.07 (m, 1H), 5.38 (dd, J = 5.2, 12.4 Hz, 1H), 4.41- |
| | | 4.30 (m, 2H), 3.71 (s, 3H), 3.68-3.60 (m, 5H), 3.26-3.20 (m, 4H), 3.18-3.16(m, |
| | | 2H), 3.12-3.08 (m, 3H), 3.06-2.99 (m, 2H), 2.96-2.86 (m, 3H), 2.82-2.78 (m, |
| | | 2H), 2.73-2.71 (m, 1H), 2.70-2.58 (m, 8H), 2.41-2.24 (m, 2H), 2.06-1.95 (m, |
| | | 1H) |
| I-1286 | 863.7 | 11.95 (s, 1H), 11.10 (s, 1H), 7.23-7.13 (m, 1H), 7.04-6.92 (m, 3H), 6.91-6.81 |
| | | (m, 1H), 6.67 (s, 1H), 6.60 (d, J = 8.8 Hz, 1H), 6.48 (s, 1H), 6.15-6.08 (m, 1H), |
| | | 5.38 (dd, J = 5.6, 12.4 Hz, 1H), 4.34 (s, 2H), 3.71 (s, 3H), 3.67-3.62 (m, 2H), 3.62 |
| | | (s, 3H), 3.26 (d, J = 0.8 Hz, 6H), 3.18-3.04 (m, 6H), 2.94-2.84 (m, 1H), 2.73- |
| | | 2.58 (m, 10H), 2.45-2.41 (m, 2H), 2.37 (d, J = 4.4 Hz, 1H), 2.30-2.20 (m, 1H), |
| | | 2.05-1.93 (m, 1H) |
| I-1314 | 913.3 | 11.99-11.92 (m, 1H), 11.11 (s, 1H), 8.56-8.47 (m, 1H), 8.29-8.18 (m, 1H), |
| | | 7.22-7.15 (m, 1H), 7.02-6.92 (m, 3H), 6.90-6.81 (m, 1H), 6.68 (s, 1H), 6.61 (dd, J = |
| | | 1.6, 8.4 Hz, 1H), 6.51-6.46 (m, 1H), 6.44-6.36 (m, 1H), 6.16-6.05 (m, 1H), 5.39 |
| | | (dd, J = 5.2, 12.0 Hz, 1H), 4.37-4.28 (m, 2H), 4.07 (q, J = 6.9 Hz, 2H), 3.74-3.69 |
| | | (m, 3H), 3.67-3.56 (m, 5H), 3.27 (s, 4H), 3.19-3.00 (m, 7H), 2.95-2.86 (m, 3H), |
| | | 2.72-2.61 (m, 8H), 2.34 (d, J = 1.8 Hz, 2H), 2.27 (d, J = 1.2 Hz, 1H), 2.07-1.95 |
| | | (m, 1H) |
| I-1463 | 992.4 | 11.99-11.88 (m, 1H), 11.10 (s, 1H), 8.00-7.94 (m, 1H), 7.65-7.59 (m, 1H), |
| | | 7.34-7.26 (m, 1H), 7.25-7.21 (m, 1H), 7.20-7.03 (m, 3H), 7.02-6.92 (m, 3H), 6.79 |
| | | (d, J = 6.4 Hz, 1H), 6.84-6.54 (m, 2H), 6.50-6.43 (m, 1H), 5.97 (s, 1H), 5.38 (dd, |
| | | J = 5.2, 12.6 Hz, 1H), 5.25-5.10 (m, 1H), 5.02-4.90 (m, 1H), 4.85-4.76 (m, 1H), |
| | | 4.67-4.55 (m, 1H), 4.47 (d, J = 5.6 Hz, 1H), 4.43-4.29 (m, 2H), 3.71 (s, 3H), |
| | | 3.68-3.58 (m, 4H), 3.56-3.45 (m, 1H), 3.26-3.19 (m, 5H), 3.18-3.11 (m, 4H), 3.10- |
| | | 2.95 (m, 3H), 2.94-2.84 (m, 1H), 2.77-2.57 (m, 9H), 2.23-1.99 (m, |
| | | 2H), 1.78-1.61 (m, 1H) |
| I-1299 | 879.3 | 11.10 (s, 1H), 8.51-8.39 (m, 1H), 7.69 (s, 1H), 7.67-7.62 (m, 1H), 7.47-7.37 (m, |
| | | 2H), 7.35-7.25 (m, 2H), 7.23-7.13 (m, 1H), 6.99 (d, J = 5.2 Hz, 2H), 6.97-6.92 |
| | | (m, 1H), 6.69 (s, 1H), 6.63 (d, J = 8.4 Hz, 1H), 6.34-6.26 (m, 1H), 5.38 (dd, J = |
| | | 5.2, 12.4 Hz, 1H), 4.41 (s, 2H), 3.72 (d, J = 5.2 Hz, 3H), 3.62 (s, 3H), 3.62-3.58 |
| | | (m, 2H), 3.28-3.26 (m, 2H), 3.25-3.18 (m, 3H), 3.18-3.10 (m, 3H), 3.05-2.95 |
| | | (m, 5H), 2.94-2.81 (m, 3H), 2.78-2.71 (m, 1H), 2.71-2.63 (m, 7H), 2.62-2.59 |
| | | (m, 1H), 2.39-2.30 (m, 1H), 2.28-2.20 (m, 1H), 2.05-1.95 (m, 1H) |
| I-1469 | 950.6 | 11.95 (s, 1H), 11.09 (s, 1H), 8.33-8.16 (m, 1H), 7.26-7.10 (m, 5H), 7.02-6.93 |
| | | (m, 3H), 6.85-6.77 (m, 1H), 6.67 (s, 1H), 6.59 (d, J = 8.0 Hz, 1H), 6.48 (d, J = 2.4 |
| | | Hz, 1H), 6.13 (s, 1H), 5.37 (dd, J = 5.6, 12.4 Hz, 2H), 4.92-4.68 (m, 1H), 4.54- |
| | | 4.37 (m, 2H), 4.35-4.10 (m, 1H), 3.78-3.68 (m, 4H), 3.62 (s, 3H), 3.26-3.25 (m, |
| | | 6H), 3.20-3.09 (m, 5H), 3.09-2.96 (m, 4H), 2.89-2.88 (m, 1H), 2.67-2.66 (m, |
| | | 8H), 2.40-2.39 (m, 1H), 2.32-2.18 (m, 1H), 2.00 (s, 1H) |
| I-1301 | 933.7 | 10.83 (s, 1H), 8.28 (d, J = 10.8 Hz, 1H), 7.67 (s, 1H), 7.44 (s, 1H), 7.35 (s, 1H), |
| | | 7.30 (d, J = 7.6 Hz, 1H), 7.23-7.12 (m, 4H), 7.01-6.91 (m, 3H), 6.68 (s, 1H), 6.64 |
| | | (d, J = 8.4 Hz, 1H), 6.33-6.27 (m, 1H), 5.47-5.26 (m, 2H), 4.93-4.67 (m, 1H), |
| | | 4.59-4.46 (m, 2H), 4.45-4.27 (m, 1H), 3.74 (s, 3H), 3.73-3.63 (m, 2H), 3.63 (s, |
| | | 3H), 3.33-3.26 (m, 4H), 3.20-3.09 (m, 8H), 3.06-3.01 (m, 2H), 2.96-2.84 (m, |
| | | 1H), 2.78-2.72 (m, 1H), 2.71-2.67 (m, 6H), 2.66-2.61 (m, 1H), 2.42-2.30 (m, |
| | | 2H), 2.10-1.98 (m, 1H) |
| I-1470 | 992.3 | 12.13 (s, 1H), 11.10 (s, 1H), 10.68 (s, 1H), 8.15-8.07 (m, 1H), 7.74-7.63 (m, 1H), |
| | | 7.18 (s, 1H), 7.14-7.08 (m, 1H), 7.06-6.93 (m, 4H), 6.60 (d, J = 8.0 Hz, 1H), 6.54 |
| | | (s, 1H), 6.48 (d, J = 2.4 Hz, 1H), 6.24-6.16 (m, 1H), 5.38 (dd, J = 5.6, 12.4 Hz, |
| | | 1H), 4.62 (q, J = 6.8 Hz, 2H), 4.46-4.29 (m, 2H), 3.69-3.56 (m, 5H), 3.47-3.41 |
| | | (m, 1H), 3.30 (s, 1H), 3.25-3.20 (m, 4H), 3.18-3.02 (m, 9H), 2.94-2.85 (m, 1H), |
| | | 2.78 (s, 3H), 2.74-2.63 (m, 4H), 2.39-2.24 (m, 2H), 2.06-1.96 (m, 1H) |
| I-1303 | 904.7 | 11.21-10.99 (m, 1H), 8.28 (dd, J = 6.8, 13.2 Hz, 1H), 7.80-7.61 (m, 1H), 7.54 |
| | | (dd, J = 6.0, 9.2 Hz, 1H), 7.48 (s, 1H), 7.44-7.38 (m, 2H), 7.31-7.15 (m, 2H), |
| | | 7.04-6.86 (m, 4H), 6.73-6.57 (m, 2H), 6.38-6.26 (m, 1H), 5.37 (dd, J = 5.6, 12.4 |
| | | Hz, 1H), 4.64-4.38 (m, 2H), 4.34-4.16 (m, 2H), 3.77-3.74 (m, J = 5.5 Hz, 1H), |
| | | 3.72-3.70 (m, J = 5.0 Hz, 3H), 3.67-3.63 (m, 1H), 3.61 (s, 3H), 3.29-3.22 (m, |
| | | 6H), 3.18-3.10 (m, 3H), 3.04-2.95 (m, 3H), 2.93-2.82 (m, 1H), 2.72-2.60 (m, |
| | | 8H), 2.42-2.28 (m, 2H), 2.07-1.94 (m, 1H) |
| I-1475 | 900.7 | 11.96 (br s, 1H), 11.10 (br s, 1H), 8.09-8.04 (m, 1H), 7.72-7.65 (m, 1H), 7.22- |
| | | 7.14 (m, 1H), 6.99 (d, J = 5.4 Hz, 2H), 6.96-6.91 (m, 1H), 6.88-6.80 (m, 1H), |
| | | 6.67 (s, 1H), 6.60 (br d, J = 8.5 Hz, 1H), 6.49 (d, J = 2.8 Hz, 1H), 6.07 (br s, 1H), |
| | | 5.37 (dd, J = 5.4, 12.5 Hz, 1H), 4.59 (td, J = 7.0, 13.7 Hz, 1H), 4.45-4.38 (m, 1H), |
| | | 4.37-4.27 (m. 2H), 3.71 (s, 3H), 3.62 (s, 3H), 3.57-3.51 (m, 1H), 3.26 (br s, 4H), |
| | | 3.21-2.98 (m, 8H), 2.95-2.84 (m, 1H), 2.76-2.63 (m, 8H), 2.61 (br d, J = 1.9 Hz, |
| | | 2H), 2.39-2.20 (m, 2H), 2.04-1.96 (m, 1H), 1.10-0.98 (m, 3H) |

^{a} The amines and acids were coupled under standard conditions at room temperature from 1-4 hrs.
^{b}HOBt added to coupling. Other bases and solvents could be employed. Final compounds were purified via standard techniques including prep-HPLC and other chromatography techniques.

### Example 4 (Method 4): Synthesis of 4-[4-(4-{2-[(1-{2-[(3aR,7aR)-4-(3-fluorophenyl)-hexahydro-2H-pyrrolo[3,2-b]pyridin-1-yl]pyridin-4-yl}piperidin-4-yl)methoxy]ethyl}piperazin-1-yl)-2-methoxyphenyl]-7-fluoro-N,N-dimethyl-6-{1-[3-(1,2,3-triazol-1-yl)propanoyl]-5,6-dihydro-2H-pyridin-3-yl}-1H-indole-2-carboxamide (I-1049)

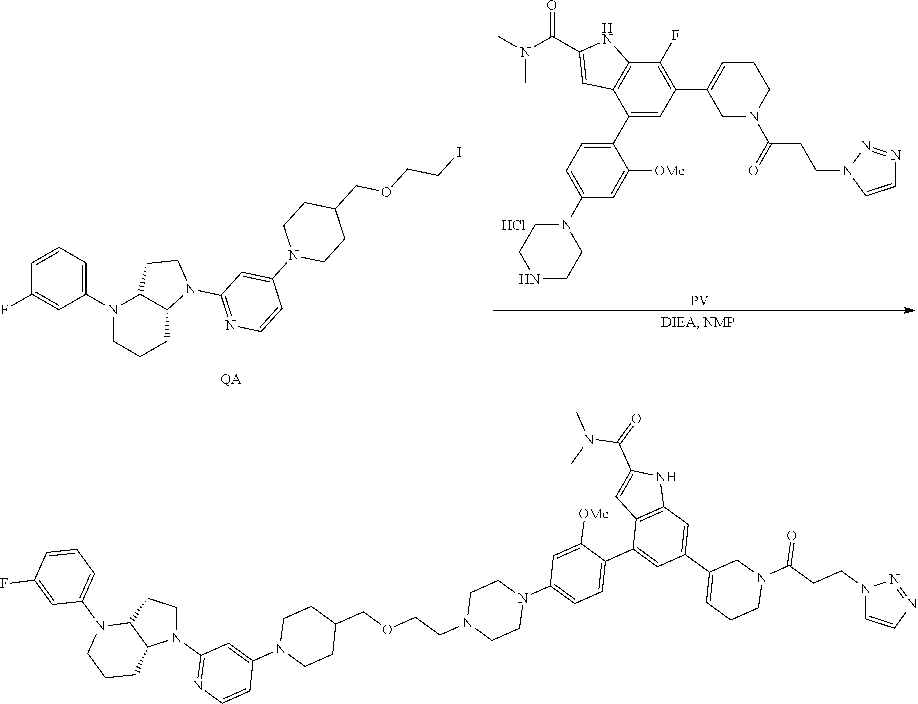

To a stirred solution of 7-fluoro-4-[2-methoxy-4-(piperazin-1-yl)phenyl]-N,N-dimethyl-6-{1-[3-(1,2,3-triazol-1-yl)propanoyl]-5,6-dihydro-2H-pyridin-3-yl}-1H-indole-2-carboxamide hydrochloride (112.87 mg, 0.177 mmol, Intermediate PV) and DIEA (0.15 mL, 0.89 mmol) in NMP (1 mL) was added 2-[(3aR,7aR)-4-(3-fluorophenyl)-hexahydro-2H-pyrrolo[3,2-b]pyridin-1-yl]-4-{4-[(2-iodoethoxy)methyl]piperidin-1-yl}pyridine (100 mg, 0.177 mmol, Intermediate QA) at rt under air atmosphere. The resulting mixture was stirred for 1 h at 110° C. under air atmosphere. On completion, the mixture was cooled to rt and concentrated under reduced pressure. The residue was purified by reverse-phase flash chromatography (column, C18 silica gel; mobile phase, MeCN in Water (10 mmol/L NH₄HCO₃), 40% to 95% gradient in 30 min; detector, UV 254 nm; desired fractions were collected at 73% B and concentrated under reduced pressure) to afford the title compound (54 mg, 29% yield) as a pink solid. ¹H NMR (400 MHz, DMSO-d₆) δ 11.97 (d, J=7.6 Hz, 1H), 8.15-8.08 (m, 1H), 7.75-7.65 (m, 2H), 7.23-7.13 (m, 2H), 6.91-6.80 (m, 1H), 6.80-6.71 (m, 2H), 6.68-6.62 (m, 1H), 6.61-6.54 (m, 1H), 6.51-6.41 (m, 2H), 6.19-6.13 (m, 1H), 6.13-6.05 (m, 1H), 5.69 (d, J=2.2 Hz, 1H), 4.67-4.57 (m, 2H), 4.49-4.39 (m, 1H), 4.36 (d, J=2.7 Hz, 1H), 4.30 (s, 1H), 4.19-4.09 (m, 1H), 3.86 (d, J=12.8 Hz, 2H), 3.70 (d, J=3.9 Hz, 3H), 3.68-3.57 (m, 2H), 3.57-3.53 (m, 1H), 3.52-3.45 (m, 1H), 3.44-3.35 (m, 1H), 3.34 (s, 2H), 3.31-3.18 (m, 8H), 3.17 (s, 1H), 3.14-3.04 (m, 4H), 2.83-2.70 (m, 3H), 2.63-2.58 (m, 3H), 2.58-2.51 (m, 3H), 2.38-2.30 (m, 1H), 2.28-2.18 (m, 2H), 2.07-1.97 (m, 1H), 1.97-1.84 (m, 11H), 1.80-1.69 (m, 3H), 1.69 (s, 1H), 1.57-1.43 (m, 1H), 1.27-1.13 (m, 4H). LC/MS (ESI, m/z): [(M+H)]⁺=1037.5.

TABLE 9
Compounds synthesized via Method 4, the coupling
of the corresponding amines and halides.

| | | | LCMS | |
|---|---|---|---|---|
| | | | (ESI+) | |
| | | | m/z | |
| I-#^{a} | Amine | Halide | (M + H)⁺ | ¹H NMR (400 MHz, DMSO-d₆) δ |
| I-1050 | PV | SB | 1140.2 | 8.15-8.07 (m, 1H), 7.73-7.65 (m, 2H), 7.17 (t, J = 7.9 |
| | | | | Hz, 2H), 6.90-6.80 (m, 1H), 6.81-6.70 (m, 2H), 6.66- |
| | | | | 6.63 (m, 1H), 6.59-6.54 (m, 1H), 6.51-6.43 (m, 2H), |
| | | | | 6.17-6.05 (m, 2H), 5.68-5.65 (m, 1H), 4.68-4.56 (m, |
| | | | | 2H), 4.46-4.38 (m, 1H), 4.38-4.26 (m, 2H), 4.18-4.08 |
| | | | | (m, 1H), 3.81 (d, J = 12.9 Hz, 2H), 3.70 (s, 3H), 3.65 (t, |
| | | | | J = 5.8 Hz, 1H), 3.62-3.55 (m, 4H), 3.55-3.52 (m, 6H), |
| | | | | 3.52-3.43 (m, 6H), 3.40-3.36 (m, 2H), 3.24-3.17 (m, |
| | | | | 5H), 313-3.03 (m, 5H), 2.82-2.66 (m, 3H), 2.62-2.56 |
| | | | | (m, 4H), 2.56-2.52 (m, 2H), 2.38-2.16 (m, 3H), 2.05- |
| | | | | 1.96 (m, 1H), 1.94-1.82 (m, 1H), 1.79-1.65 (m, 3H), |
| | | | | 1.61-1.39 (m, 4H), 1.25-1.08 (m, 3H) |
| I-1051 | PV | RS | 1801.3 | 11.95 (d, J = 8.5 Hz, 1H), 8.11 (d, J = 11.2 Hz, 1H), |
| | | | | 7.75-7.56 (m, 2H), 7.22-7.10 (m, 2H), 6.84-6.41 |
| | | | | (m, 5H), 6.17-6.06 (m, 2H), 5.68 (d, J = 2.1 Hz, 1H), |
| | | | | 4.62 (dt, J = 9.8, 6.8 Hz, 2H), 4.41-4.25 (m, 2H), |
| | | | | 4.18-3.84 (m, 2H), 3.68-3.43 (m, 8H), 3.41-2.97 (m, |
| | | | | 20H), 2.75-2.39 (m, 13H), 2.34-2.11 (m, 2H), 2.02- |
| | | | | 1.60 (m, 5H), 1.50-1.03 (m, 6H) |
| I-1052 | PV | RU | 1236.5 | Methanol-d₄) δ 8.51 (s, 2H), 8.01 (d, J = 9.9 Hz, 1H), |
| | | | | 7.83-7.76 (m, 1H), 7.70 (d, J = 11.4 Hz, 1H), 7.38-7.29 |
| | | | | (m, 2H), 7.28-7.19 (m, 4H), 7.17-7.13 (m, 1H), 7.13- |
| | | | | 7.03 (m, 2H), 6.98-6.89 (m, 1H), 6.85-6.79 (m, 1H), |
| | | | | 6.74-6.60 (m, 4H), 6.14 (d, J = 10.2 Hz, 1H), 5.29 (q, |
| | | | | J = 6.9 Hz, 1H), 4.79-4.74 (m, 2H), 4.67-4.61 (m, 2H), |
| | | | | 4.48-4.39 (m, 2H), 3.80-3.71 (m, 6H), 3.68-3.56 (m, |
| | | | | 9H), 3.45-3.36 (m, 5H), 3.22-3.10 (m, 3H), 3.05-2.92 |
| | | | | (m, 6H), 2.88-2.82 (m, 2H), 2.64-2.57 (m, 2H), 2.43- |
| | | | | 2.36 (m, 2H), 1.87 (s, 3H) |
| I-1053 | PV | RW | 1206.8 | (Methanol-d₄) δ 8.07-7.97 (m, 1H), 7.79 (d, J = 6.5 |
| | | | | Hz, 1H), 7.70 (d, J = 10.5 Hz, 1H), 7.32 (d, J = 7.8 Hz, |
| | | | | 2H), 7.28-7.20 (m, 3H), 7.14 (m, J = 17.6, 7.4 Hz, |
| | | | | 1H), 7.02 (d, J = 7.8 Hz, 1H), 6.91 (m, J = 6.3, 4.2 Hz, |
| | | | | 1H), 6.82 (td, J = 9.0, 4.3 Hz, 1H), 6.76-6.59 (m, 3H), |
| | | | | 6.13 (d, J = 10.0 Hz, 1H), 5.30 (d, J = 6.8 Hz, 2H), 4.63 |
| | | | | (s, 9H), 4.46 (s, 1H), 4.37 (s, 1H), 3.80-3.72 (m, 3H), |
| | | | | 3.67 (q, J = 4.0, 2.7 Hz, 2H), 3.47-3.39 (m, 2H), 3.20- |
| | | | | 3.12 (m, 3H), 2.88-2.78 (m, 3H), 2.76 (t, J = 5.3 Hz, |
| | | | | 1H), 2.69-2.59 (m, 3H), 2.37 (d, J = 16.8 Hz, 1H), |
| | | | | 1.88 (t, J = 3.1 Hz, 2H), 1.83 (d, J = 3.1 Hz, 1H), 1.31 |
| | | | | (d, J = 3.5 Hz, 2H), 0.91 (d, J = 9.7 Hz, 1H) |
| I-1054 | PV | SD | 1183.7 | 11.99-11.94 (m, 1H), 8.14-8.09 (m, 1H), 7.71-7.66 (m, |
| | | | | 2H), 7.20-7.13 (m, 2H), 6.89-6.80 (m, 1H), 6.80-6.72 |
| | | | | (m, 2H), 6.64 (s, 1H), 6.59-6.53 (m, 1H), 6.50-6.42 (m, |
| | | | | 2H), 6.15-6.05 (m, 2H), 5.65 (s, 1H), 4.66-4.58 (m, |
| | | | | 2H), 4.47-4.39 (m, 1H), 4.38-1.28 (m, 2H), 4.15-4.07 |
| | | | | (m, 2H), 3.71-3.67 (m, 3H), 3.67-3.57 (m, 3H), 3.57- |
| | | | | 3.48 (m, 14H), 3.48-3.42 (m, 3H), 3.41-3.36 (m, 3H), |
| | | | | 3.35-3.34 (m, 6H), 3.22-3.16(m, 5H), 3.13-3.06 (m, |
| | | | | 3H), 2.89-2.73 (m, 2H), 2.61-2.54 (m, 3H), 2.37-2.30 |
| | | | | (m, 1H), 2.29-2.16 (m, 2H), 2.06-1.84 (m, 2H), 1.81- |
| | | | | 1.71 (m, 3H), 1.67-1.37 (m, 8H) |
| I-1055 | PV | QV | 1092.2 | 12.00-11.92 (m, 1H), 8.20 (s, 1H, HCOOH), 8.11 (d, J = |
| | | | | 10.5 Hz, 1H), 7.75-7.64 (m, 2H), 7.23-7.12 (m, 2H), |
| | | | | 6.90-6.81 (m, 1H), 6.80-6.72 (m, 2H), 6.68-6.62 (m, |
| | | | | 1H), 6.61-6.54 (m, 1H), 6.51-6.43 (m, 2H), 6.18-6.05 |
| | | | | (m, 2H), 5.69-5.66 (m, 1H), 4.67-4.57 (m, 2H), 4.48- |
| | | | | 4.38 (m, 1H), 4.38-4.24 (m, 2H), 4.18-4.08 (m, 1H), |
| | | | | 3.83 (d, J = 12.8 Hz, 2H), 3.70 (d, J = 4.0 Hz, 3H), |
| | | | | 3.67-3.55 (m, 2H), 3.54-3.45 (m, 2H), 3.35-3.27 (m, |
| | | | | 6H), 3.25-3.13 (m, 7H), 3.12-2.97 (m, 6H), 2.83-2.63 |
| | | | | (m, 3H), 2.37-2.29 (m, 3H), 2.29-2.15 (m, 2H), 2.06- |
| | | | | 1.83 (m, 2H), 1.80-1.63 (m, 3H), 1.52-1.36 (m, 3H), |
| | | | | 1.35-1.17 (m, 12H), 1.15-1.03 (m, 2H) |
| I-1056 | PV | QQ | 1095.4 | 11.96 (d, J = 8.6 Hz, 1H), 8.11 (dd, J = 10.6, 1.0 Hz, |
| | | | | 1H), 7.72-7.64 (m, 2H), 7.20-7.10 (m, 2H), 6.90- |
| | | | | 6.76 (m, 2H), 6.80-6.72 (m, 1H), 6.62 (s, 1H), 6.52 (d, |
| | | | | J = 8.5 Hz, 1H), 6.49-6.45 (m, 1H), 6.41 (ddd, J = |
| | | | | 10.0, 7.9, 2.3 Hz, 1H), 6.16-6.06 (m, 2H), 5.68 (d, J = |
| | | | | 2.2 Hz, 1H), 4.67-4.57 (m, 2H), 4.36 (s, 1H), 4.32- |
| | | | | 4.21 (m, 2H), 4.04 (d, J = 8.5 Hz, 1H), 3.69 (d, J = 3.6 |
| | | | | Hz, 3H), 3.67-3.53 (m, 3H), 3.56-3.43 (m, 3H), 3.23 |
| | | | | (s, 3H), 3.17 (d, J = 5.5 Hz, 5H), 3.11 (d, J = 7.1 Hz, |
| | | | | 1H), 3.08 (s, 5H), 3.04 (d, J = 11.1 Hz, 1H), 2.99 (d, J = |
| | | | | 10.0 Hz, 1H), 2.76 (s, 1H), 2.55 (dd, J = 16.7, 5.6 Hz, |
| | | | | 4H), 2.27 (s, 1H), 2.20 (s, 1H), 1.61 (d, J = 11.7 Hz, |
| | | | | 1H), 1.52 (s, 9H), 1.35 (t, J = 11.7 Hz, 1H) |
| I-1057 | PV | QP | 1095.4 | 11.96 (d, J = 8.6 Hz, 1H), 8.11 (dd, J = 10.6, 1.0 Hz, |
| | | | | 1H), 7.72-7.64 (m, 2H), 7.20-7.10 (m, 2H), 6.90- |
| | | | | 6.76 (m, 2H), 6.80-6.72 (m, 1H), 6.62 (s, 1H), 6.52 (d, |
| | | | | J = 8.5 Hz, 1H), 6.49-6.45 (m, 1H), 6.41 (ddd, J = |
| | | | | 10.0, 7.9, 2.3 Hz, 1H), 6.16-6.06 (m, 2H), 5.68 (d, J = |
| | | | | 2.2 Hz, 1H), 4.67-4.57 (m, 2H), 4.36 (s, 1H), 4.32- |
| | | | | 4.21 (m, 2H), 4.04 (d, J = 8.5 Hz, 1H), 3.69 (d, J = 3.6 |
| | | | | Hz, 3H), 3.67-3.53 (m, 3H), 3.56-3.43 (m, 3H), 3.23 |
| | | | | (s, 3H), 3.17 (d, J = 5.5 Hz, 5H), 3.11 (d, J = 7.1 Hz, |
| | | | | 1H), 3.08 (s, 5H), 3.04 (d, J = 11.1 Hz, 1H), 2.99 (d, J = |
| | | | | 10.0 Hz, 1H), 2.76 (s, 1H), 2.55 (dd, J = 16.7, 5.6 Hz, |
| | | | | 4H), 2.27 (s, 1H), 2.20 (s, 1H), 1.61 (d, J = 11.7 Hz, |
| | | | | 1H), 1.52 (s, 9H), 1.35 (t, J = 11.7 Hz, 1H) |
| I-1058 | PV | QH | 1051.6 | 11.98-11.88 (m, 1H), 8.20 (s, 1H), 8.11 (dd, J = 10.3, |
| | | | | 1.0 Hz, 1H), 7.71-7.64 (m, 2H), 7.19-7.11 (m, 2H), |
| | | | | 6.88-6.74 (m, 3H), 6.63 (d, J = 2.3 Hz, 1H), 6.55 (dd, |
| | | | | J = 8.5, 2.2 Hz, 1H), 6.49-6.39 (m, 2H), 6.17 (dd, J = |
| | | | | 6.3, 2.1 Hz, 1H), 6.14-6.06 (m, 1H), 5.70 (d, J = 2.2 |
| | | | | Hz, 1H), 4.66-4.57 (m, 2H), 4.38-4.24 (m, 3H), 4.07 |
| | | | | (q, J = 7.2 Hz, 1H), 3.72-3.67 (m, 3H), 3.67-3.63 |
| | | | | (m, 1H), 3.62-3.56 (m, 2H), 3.56-3.51 (m, 1H), 3.48 |
| | | | | (t, J = 5.7 Hz, 2H), 3.42-3.33 (m, 2H), 3.29-3.23 (m, |
| | | | | 5H), 3.22-3.14 (m, 6H), 3.12-3.00 (m, 7H), 2.84- |
| | | | | 2.71 (m, 1H), 2.61-2.55 (m, 4H), 2.55-2.51 (m, 2H), |
| | | | | 2.37-2.31 (m, 1H), 2.29-2.14 (m, 2H), 1.67-1.59 |
| | | | | (m, 2H), 1.57-1.49 (m, 7H), 1.48-1.43 (m, 1H), |
| | | | | 1.42-1.34 (m, 1H) |
| I-1059 | PV | SF | 1253.5 | 11.94 (d, J = 8.6 Hz, 1H), 8.50 (dd, J = 13.4, 8.4 Hz, |
| | | | | 1H), 8.11 (d, J = 10.4 Hz, 1H), 7.85 (d, J = 7.2 Hz, 1H), |
| | | | | 7.72-7.66 (m, 1H), 7.43 (d, J = 5.7 Hz, 1H), 7.38-7.27 |
| | | | | (m, 5H), 7.17 (d, J = 8.3 Hz, 1H), 6.98 (d, J = 7.9 Hz, |
| | | | | 2H), 6.86 (dd, J = 20.8, 6.2 Hz, 1H), 6.76-6.68 (m, 1H), |
| | | | | 6.66-6.55 (m, 3H), 6.53-6.44 (m, 1H), 6.11 (d, J = 11.7 |
| | | | | Hz, 1H), 5.46-5.35 (m, 1H), 4.68-4.57 (m, 2H), 4.43- |
| | | | | 4.29 (m, 2H), 3.92 (s, 3H), 3.76-3.69 (m, 4H), 3.67-3.56 |
| | | | | (m, 4H), 3.55-3.49 (m, 1H), 3.36-3.18 (m, 6H), 3.15- |
| | | | | 3.02 (m, 3H), 2.99-2.91 (m, 3H), 2.75-2.64 (m, 1H), |
| | | | | 2.37-2.23 (m, 6H), 1.88-1.79 (m, 4H), 1.53-1.40 (m, |
| | | | | 5H), 1.35-1.14 (m, 6H) |
| I-1060 | PV | QS | 1281.5 | 11.99-11.91 (m, 1H), 8.56-8.45 (m, 1H), 8.11 (d, J = |
| | | | | 10.6 Hz, 1H), 7.85 (d, J = 7.3 Hz, 1H), 7.69 (d, J = 12.0 |
| | | | | Hz, 1H), 7.43 (d, J = 5.7 Hz, 1H), 7.39-7.25 (m, 5H), |
| | | | | 7.17 (d, J = 8.4 Hz, 1H), 6.97 (d, J = 7.9 Hz, 2H), 6.80- |
| | | | | 6.61 (m, IH), 6.75-6.53 (m, 4H), 6.52-6.48 (m, 1H), |
| | | | | 6.15-6.07 (m, 1H), 5.47-5.36 (m, 1H), 4.68-4.57 (m, |
| | | | | 2H), 4.37 (s, 1H), 4.31 (s, 1H), 3.91 (s, 3H), 3.76-3.56 |
| | | | | (m, 6H), 3.20-3.13 (m, 6H), 3.22 (s, 3H), 3.09 (q, J = |
| | | | | 7.2 Hz, 2H), 2.97 (d, J = 9.3 Hz, 3H), 2.53 (s, 1H), |
| | | | | 2.57-2,50 (m, 4H), 2.50-2,46 (m, 2H), 2.37-2.25 (m, |
| | | | | 4H), 1.81 (d, J = 44.0 Hz, 3H), 1.38-1.36 (m, 4H), |
| | | | | 1.35-1.26 (m, 6H), 1.18-1.15 (s, 2H) |
| I-1061 | PV | SJ | 1346.0 | 11.94 (s, 1H), 8.63 (s, 1H), 8.11 (d, J = 10.9 Hz, 1H), |
| | | | | 7.93-7.84 (m, 1H), 7.68 (d, J = 12.1 Hz, 1H), 7.45 (s, |
| | | | | 1H), 7.41-7.28 (m, 5H), 7.16 (d, J = 8.3 Hz, 1H), |
| | | | | 6.91-6.75 (m, 2H), 6.75-6.69 (m, 2H), 6.69-6.53 (m, |
| | | | | 3H), 6.48 (t, J = 2.9 Hz, 1H), 6.14-6.06 (m, 1H), 5.40- |
| | | | | 5.42 (m, 1H), 4.61-4.63 (m, 2H), 4.36 (s, 1H), 4.30 (s, |
| | | | | 1H), 4.03-4.05 (m, 2H), 3.85 (s, 3H), 3.70 (d, J = 3.8 |
| | | | | Hz, 7H), 3.68-3.49 (m, 15H), 3.35-3.30 (m, 9H), |
| | | | | 3.21 (t, J = 4.9 Hz, 4H), 3.08-3.10 (m, 2H), 3.04 (s, |
| | | | | 3H), 2.97 (dd, J = 7.7, 2.2 Hz, 3H), 2.62-2.51 (m, 6H), |
| | | | | 2.34 (s, 1H), 2.27 (s, 1H), 2.05-1.93 (m, 1H), 1.89 (d, |
| | | | | J = 2.5 Hz, 2H), 1.75 (d, J = 2.6 Hz, 2H), 1.27 (s, 1H), |
| | | | | 1.24 (s, 3H) |
| I-1062^{c} | PV | SI | 1303.5 | 8.01 (d. J = 10.3 Hz, 1H), 7.74-7.63 (m, 2H), 7.44 (dd, |
| | | | | J = 8.8, 2.2 Hz, 2H), 7.38-7.16 (m, 5H), 6.92 (t, J = 6.2 |
| | | | | Hz, 1H), 6.75-6.54 (m, 7H), 6.14 (d, J = 13.6 Hz, 1H), |
| | | | | 5.58-5.49 (m, IH), 4.83-4.72 (m, 3H), 4.48-4.39 (m, |
| | | | | 2H), 4.09 (t, J = 4.3 Hz, 2H), 3.85 (s, 3H), 3.82 (t, J = |
| | | | | 4.6 Hz, 2H), 3.78-3.54 (m, 14H), 3.35-3.27( m, 4H), |
| | | | | 3.21-3.08 (m, 4H), 2.89 (dd, J = 8.5, 1.7 Hz, 3H), 2.82- |
| | | | | 2.76 (m, 4H), 2.73-2.67 (m, 2H), 2.42-2.35 (m, 2H), |
| | | | | 1.86 (dd, J = 14.8, 2.6 Hz, 3H), 1.37-1.29(m, 2H) |
| I-1063 | PV | QI | 1051.6 | 11.98-11.88 (m, 1H), 8.20 (s, 1H), 8.11 (dd, J = 10.3, |
| | | | | 1.0 Hz, 1H), 7.71-7.64 (m, 2H), 7.19-7.11 (m, 2H), |
| | | | | 6.88-6.74 (m, 3H), 6.63 (d, J = 2.3 Hz, 1H), 6.55 (dd, |
| | | | | J = 8.5, 2.2 Hz, 1H), 6.49-6.39 (m, 2H), 6.17 (dd, J = |
| | | | | 6.3, 2.1 Hz, 1H), 6.14-6.06 (m, 1H), 5.70 (d, J = 2.2 |
| | | | | Hz, 1H), 4.66-4.57 (m, 2H), 4.38-4.24 (m, 3H), 4.07 |
| | | | | (q, J = 7.2 Hz, 1H), 3.72-3.67 (m, 3H), 3.67-3.63 |
| | | | | (m, IH), 3.62-3.56 (m, 2H), 3.56-3.51 (m, IH), 3.48 |
| | | | | (t, J = 5.7 Hz, 2H), 3.42-3.33 (m, 2H), 3.29-3.23 (m, |
| | | | | 5H), 3.22-3.14 (m, 6H), 3.12-3.00 (m, 7H), 2.84- |
| | | | | 2.71 (m, 1H), 2.61-2.55 (m, 4H), 2.55-2.51 (m, 2H), |
| | | | | 2.37-2.31 (m, 1H), 2.29-2.14 (m, 2H), 1.67-1.59 |
| | | | | (m, 2H), 1.57-1.49 (m, 7H), 1.48-1.43 (m, 1H), |
| | | | | 1.42-1.34 (m, 1H) |
| I-1064 | PV | QJ | 1051.6 | 11.98-11.88 (m, 1H), 8.20 (s, 1H), 8.11 (dd, J = 10.3, |
| | | | | 1.0 Hz, 1H), 7.71-7.64 (m, 2H), 7.19-7.11 (m, 2H), |
| | | | | 6.88-6.74 (m, 3H), 6.63 (d, J = 2.3 Hz, 1H), 6.55 (dd, |
| | | | | J = 8.5, 2.2 Hz, 1H), 6.49-6.39 (m, 2H), 6.17 (dd, J = |
| | | | | 6.3, 2.1 Hz, 1H), 6.14-6.06 (m, 1H), 5.70 (d, J = 2.2 |
| | | | | Hz, 1H), 4.66-4.57 (m, 2H), 4.38-4.24 (m, 3H), 4.07 |
| | | | | (q, J = 7.2 Hz, 1H), 3.72-3.67 (m, 3H), 3.67-3.63 |
| | | | | (m, 1H), 3.62-3.56 (m, 2H), 3.56-3.51 (m, 1H), 3.48 |
| | | | | (t, J = 5.7 Hz, 2H), 3.42-3.33 (m, 2H), 3.29-3.23 (m, |
| | | | | 5H), 3.22-3.14 (m, 6H), 3.12-3.00 (m, 7H), 2.84- |
| | | | | 2.71 (m, 1H), 2.61-2.55 (m, 4H), 2.55-2.51 (m, 2H), |
| | | | | 2.37-2.31 (m, 1H), 2.29-2.14 (m, 2H), 1.67-1.59 |
| | | | | (m, 2H), 1.57-1.49 (m, 7H), 1.48-1.43 (m, 1H), |
| | | | | 1.42-1.34 (m, 1H) |
| I-1065 | PV | QK | 1051.6 | 11.98-11.88 (m, 1H), 8.20 (s, 1H), 8.11 (dd, J = 10.3, |
| | | | | 1.0 Hz, 1H), 7.71-7.64 (m, 2H), 7.19-7.11 (m, 2H), |
| | | | | 6.88-6.74 (m, 3H), 6.63 (d, J = 2.3 Hz, 1H), 6.55 (dd, |
| | | | | J = 8.5, 2.2 Hz, 1H), 6.49-6.39 (m, 2H), 6.17 (dd, J = |
| | | | | 6.3, 2.1 Hz, 1H), 6.14-6.06 (m, 1H), 5.70 (d, J = 2.2 |
| | | | | Hz, 1H), 4.66-4.57 (m, 2H), 4.38-4.24 (m, 3H), 4.07 |
| | | | | (q, J = 7.2 Hz, 1H), 3.72-3.67 (m, 3H), 3.67-3.63 |
| | | | | (m, 1H), 3.62-3.56 (m, 2H), 3.56-3.51 (m, 1H), 3.48 |
| | | | | (t, J = 5.7 Hz, 2H), 3.42-3.33 (m, 2H), 3.29-3.23 (m, |
| | | | | 5H), 3.22-3.14 (m, 6H), 3.12-3.00 (m, 7H), 2.84- |
| | | | | 2.71 (m, 1H), 2.61-2.55 (m, 4H), 2.55-2.51 (m, 2H), |
| | | | | 2.37-2.31 (m, 1H), 2.29-2.14 (m, 2H), 1.67-1.59 |
| | | | | (m, 2H), 1.57-1.49 (m, 7H), 1.48-1.43 (m, 1H), |
| | | | | 1.42-1.34 (m, 1H) |
| I-1066^{b} | PV | PU | 1277.5 | Chloroform-d) δ 12.03-11.91 (m, 1H), 8.67-7.58 (m, |
| | | | | 1H), 8.16-8.09 (m, 1H), 7.81 (d, J = 8.3 Hz, 1H), |
| | | | | 7.71-7.66 (m, 1H), 7.43-7.39 (m, 1H), 7.36-7.20 (m, |
| | | | | 5H), 7.17-7.11 (m, 3H), 6.90-6.78 (m, 2H), 6.68- |
| | | | | 6.57 (m, 3H), 6.49-6.47 (m, 1H), 5.55-5.46 (m, 1H), |
| | | | | 4.79-4.61 (m, 2H), 4.40-429 (m, 2H), 3.97 (s, 3H), |
| | | | | 3.75-3.61 (m, 7H), 3.32-3.02 (m, 12H), 2.97 (d, J = |
| | | | | 11.1 Hz, 3H), 2.43 (d, J = 7.0 Hz, 2H), 2,41-2.39 (m, |
| | | | | 2H), 2,37-2.20 (m, 6H), 1.97-1.83 (m, 3H), 1.63- |
| | | | | 1.52 (m, 6H), 1.55-1.45 (m, 3H). |
| I-1067 | PV | RF | 1339.4 | 11.94 (d, J = 8.6 Hz, 1H), 8.50 (dd, J = 13.1, 8.3 Hz, |
| | | | | 1H), 8.11 (d, J = 10.2 Hz, 1H), 7.85 (d, J = 7.3 Hz, 1H), |
| | | | | 7.69 (d, J = 11.8 Hz, 1H), 7.44 (d, J = 5.9 Hz, 1H), |
| | | | | 7.39-7.26 (m, 5H), 7.17 (d, J = 8.3 Hz, 1H), 6.96 (d, J = |
| | | | | 7.8 Hz, 2H), 6.86 (dd, J = 20.4, 6.2 Hz, 1H), 6.72-6.52 |
| | | | | (m, 4H), 6.49 (t, J = 2.8 Hz, 1H), 6.11 (d, J = 11.7 Hz, |
| | | | | 1H), 5.49-5.35 (m, 1H), 4.66-4.60 (m, 2H), 4.37-4.29 |
| | | | | (m, 2H), 3.91 (s, 3H), 3.71 (d, J = 3.7 Hz, 3H), 3.68- |
| | | | | 3.56 (m, 4H), 3.28-3.02 (m, 11H), 2.97 (d, J = 9.4 Hz, |
| | | | | 3H), 2.57-2.52 (m, 4H), 2.49-2.42 (m, 2H), 2.38-2.27 |
| | | | | (m, 4H), 1.81 (dd, J = 52.7, 2.5 Hz, 3H), 1.47-1.39 (m, |
| | | | | 4H), 1.33-1.25 (m, 15H), 1.22-1.13 (m, 2H) |
| I-1068 | PV | RD | 1310.4 | 11.95 (d, J = 8.8 Hz, 1H), 8.51 (dd, J = 13.6, 8.4 Hz, |
| | | | | 1H), 8.11 (d, J = 10.7 Hz, 1H), 7.85 (d, J = 7.4 Hz, 1H), |
| | | | | 7.69 (d, J = 12.0 Hz, 1H), 7.43 (d, J = 5.7 Hz, 1H), |
| | | | | 7.40-7.26 (m, 6H), 7.17 (d, J = 8.3 Hz, 1H), 6.96 (d, J = |
| | | | | 7.8 Hz, 2H), 6.86 (dd, J = 21.0, 6.1 Hz, 1H), 6.73-6.54 |
| | | | | (m, 4H), 6.48 (d, J = 2.8 Hz, 1H), 6.11 (d, J = 11.5 Hz, |
| | | | | 1H), 5.46-5.35 (m, 1H), 4.67-4.59 (m, 2H), 4.33 (d, |
| | | | | J = 23.1 Hz, 2H), 3.91 (s, 3H), 3.70 (d, J = 3.9 Hz, 3H), |
| | | | | 3.67-3.54 (m, 4H), 3.22 (s, 6H), 3.09 (q, J = 7.3 Hz, |
| | | | | 5H), 2.97 (d, J = 9.3 Hz, 3H), 2.45 (d, J = 7.3 Hz, 2H), |
| | | | | 2.31 (dd, J = 17.3, 10.4 Hz, 5H), 1.87 (d, J = 2.5 Hz, |
| | | | | 2H), 1.74 (d, J = 2.5 Hz, 1H), 1.44 (dd, J = 15.2, 7.7 |
| | | | | Hz, 4H), 1.27 (t, J = 12.4 Hz, 12H), 1.16 (s, 2H) |
| I-1069 | PV | QX | 1183.7 | 12.00-11.92 (m, 1H), 8.20 (s, 1H, HCOOH), 8.11 (d, J = |
| | | | | 10.5 Hz, 1H), 7.88 (d, J = 5.4 Hz, 1H), 7.69 (d, J = |
| | | | | 12.2 Hz, 1H), 7.23-7.14 (m, 2H), 6.89-6.81 (m, 1H), |
| | | | | 6.81-6.72 (m, 2H), 6.66-6.62 (m, 1H), 6.59-6.53 (m, |
| | | | | 1H), 6.53-6.46 (m, 3H), 6.13-6.05 (m, 1H), 4.68-4.58 |
| | | | | (m, 2H), 4.45-4.38 (m, 1H), 4.37-4.28 (m, 2H), 4.25- |
| | | | | 4.18 (m, 1H), 3.76-3.62 (m, 7H), 3.58-3.48 (m, 17H), |
| | | | | 3.23-3.16 (m, 7H), 3.12-2.98 (m, 9H), 2.92-2.85 (m, |
| | | | | 2H), 2.80-2.69 (m, 3H), 2.62-2.55 (m, 4H), 2.38-2.23 |
| | | | | (m, 2H), 2.05-1.95 (m, 1H), 1.83-1.71 (m, 2H), 1.67- |
| | | | | 1.45 (m, 9H) |
| I-1070 | PV | RI | 1306.3 | 12.02-11.95 (m, 1H), 9.65 (d, J = 64.2 Hz, 1H), 8.58 |
| | | | | (d, J = 18.2 Hz, 1H), 8.11 (d, J = 9.7 Hz, 1H), 7.91 (d, |
| | | | | J = 9.5 Hz, 1H), 7.73-7.60 (m, 1H), 7.44 (d, J = 6.8 Hz, |
| | | | | 1H), 7.34 (s, 3H), 7.27-7.21 (m, 3H), 7.07 (dd, J = |
| | | | | 50.4, 4.1 Hz, 1H), 6.92-6.72 (m, 3H), 6.70-6.54 (m, |
| | | | | 1H), 6.47 (s, 1H), 6.11 (d, J = 10.0 Hz, 1H), 5.69-5.12 |
| | | | | (m, 1H), 4.63 (q, J = 7.2 Hz, 2H), 4.44-4.33 (m, 3H), |
| | | | | 3.98-3.91 (m, 4H), 3.86-3.29 (m, 15H), 3.04 (dd, J = |
| | | | | 52.4, 6.6 Hz, 12H),2.92(m, 3H), 2.31 (d, J = 29.9 Hz, |
| | | | | 2H), 2.18 (s, 1H), 2.08 (d, J = 13.5 Hz, 1H), 2.02-1.39 |
| | | | | (m, 4H), 1.26 (d, J = 17.1 Hz, 2H), 0.86 (s, 1H) |
| I-1071 | PV | RQ | 1228.7 | 11.96 (d, J = 9.0 Hz, 1H), 8.59 (d, J = 8.7 Hz, 1H), 8.11 |
| | | | | (dd, J = 10.7, 1.0 Hz, 1H), 7.94 (q, J = 2.8, 2.2 Hz, 1H), |
| | | | | 7.68 (dd, J = 12.1, 1.0 Hz, 1H), 7.44 (dd, J = 6.5, 2.0 |
| | | | | Hz, 1H), 7.42-7.26 (m, 4H), 7.17 (d, J = 8.4 Hz, 1H), |
| | | | | 6.96 (d, J = 7.9 Hz, 2H), 6.85 (dd, J = 21.2, 6.2 Hz, |
| | | | | 1H), 6.75-6.53 (m, 4H), 6.48 (t, J = 2.8 Hz, 1H), 6.10 |
| | | | | (d, J = 11.7 Hz, 1H), 5.30-5.13 (m, 1H), 4.62 (dt, J = |
| | | | | 9.5, 6.9 Hz, 2H), 4.36 (s, 1H), 4.30 (s, 1H), 3.91 (s, |
| | | | | 3H), 3.70 (d, J = 4.1 Hz, 3H), 3.67-3.48 (m, 2H), 3.21 |
| | | | | (s, 5H), 3.14-2.96 (m, 3H), 2.55-2.42 (m, 3H), 2.30 |
| | | | | (d, J = 30.8 Hz, 3H), 1.89 (d, J = 2.5 Hz, 1H), 1.76 (d, |
| | | | | J = 2.5 Hz, 1H), 1.50-1.39 (m, 1H), 1.32-1.26 (m, |
| | | | | 6H), 1.23 (s, 1H), 1.16 (s, 2H) |

^{a}Either Iodides or bromides were used for the coupling, where the reaction was run from 80-110° C. for 1-16 hrs. Purification of the final compounds was performed under a variety of standard techniques.
^{b}CDCl3-d was employed as the NMR solvent.
^{c}MeOD-d₄ employed as NMR solvent.

TABLE 10
Compounds synthesized via Method 4, the coupling of corresponding amines and halides.

| | LCMS | |
|---|---|---|
| | (ESI+) | |
| | m/z | |
| I-# | (M + H)⁺ | ¹H NMR (400 MHz, DMSO-d₆) δ |
| T- | 849.5 | 11.95 (d, J = 7.6 Hz, 1H), 11.10 (s, 1H), 7.18 (d, J = 8.4 Hz, 1H), 6.99 (d, J = 4.8 Hz, |
| 1287 | | 2H), 6.96-6.90 (m, 1H), 6.90-6.82 (m, 1H), 6.67 (s, 1H), 6.60 (d, J = 8.8 Hz, 1H), |
| | | 6.48 (s, 1H), 6.10 (s, 1H), 5.38 (dd, J = 5.6, 12.8 Hz, 1H), 4.39-4.29 (m, 2H), 3.72 (s, |
| | | 3H), 3.65-3.56 (m, 1H), 3.62 (s, 3H), 3.60-3.57 (m, 1H), 3.47-3.36 (m, 2H), 3.27- |
| | | 3.22 (m, 6H), 3.16-3.02 (m, 6H), 2.94-2.86 (m, 1H), 2.72-2.63 (m, 8H), 2.38- |
| | | 2.27 (m, 2H), 2.05-1.95 (m, 1H) |
| I- | 900.3 | 11.94 (d, J = 7.6 Hz, 1H), 11.07 (s, 1H), 8.15-8.08 (m, 1H), 7.70-7.65 (m, 1H), 7.64- |
| 1379 | | 7.58 (m, 1H), 7.15 (dd, J = 8.4, 14.5 Hz, 2H), 7.04 (d, J = 7.2 Hz, 1H), 6.89-6.80 |
| | | (m, 2H), 6.67 (s, 1H), 6.60 (dd, J = 2.0, 8.4 Hz, 1H), 6.47 (s, 1H), 6.14-6.07 (m, 1H), |
| | | 5.07-5.03 (m, 1H), 4.66-4.57 (m, 2H), 4.38-4.27 (m, 2H), 3.73-3.69 (m, 3H), |
| | | 3.66-3.57 (m, 2H), 3.27-3.26 (m, 4H), 3.20-3.01 (m, 9H), 2.90-2.81 (m, 1H), |
| | | 2.71-2.58 (m, 7H), 2.57-2.53 (m, 2H), 2.35-2.25 (m, 2H), 2.06-1.96 (m, 1H) |
| I- | 849.3 | 11.95 (s, 1H), 11.10 (s, 1H), 7.17 (d, J = 8.0 Hz, 1H), 7.04-6.91 (m, 3H), 6.90-6.81 |
| 1285 | | (m, 1H), 6.68 (s, 1H), 6.63-6.56 (m, 1H), 6.49 (s, 1H), 6.10 (d, J = 8.8 Hz, 1H), |
| | | 5.39-5.35 (m, 1H), 4.54-4.38 (m, 1H), 4.38-4.29 (m, 2H), 3.72 (s, 3H), 3.62 (s, 5H), |
| | | 3.26-3.21 (m, 4H), 3.19-3.10 (m, 5H), 3.06 (d, J = 4.4 Hz, 2H), 3.00-2.83 (m, 2H), |
| | | 2.78-2.58 (m, 9H), 2.46-2.32 (m, 4H), 2.26 (d, J = 2.4 Hz, 1H), 2.05-1.95 (m, 1H), |
| | | 1.74-1.61 (m, 2H) |
| I- | 900.4 | 11.41 (s, 1H), 11.10 (s, 1H), 8.15-8.00 (m, 1H), 7.73-7.60 (m, 1H), 7.30-7.12 (m, |
| 1465 | | 2H), 7.01-6.93 (m, 3H), 6.91-6.84 (m, 1H), 6.70-6.65 (m, 1H), 6.63-6.56 (m, |
| | | 1H), 6.51-6.44 (m, 1H), 5.32 (d, J = 2.8 Hz, 1H), 4.97-4.70 (m, 1H), 4.70-4.44 (m, |
| | | 3H), 4.05-3.94 (m, 1H), 3.70 (d, J = 7.2 Hz, 3H), 3.62 (s, 3H), 3.27 (s, 6H), 3.18- |
| | | 3.09 (m, 8H), 2.94-2.86 (m, 2H), 2.67 (s, 8H), 2.39-2.24 (m, 2H), 2.06-1.93 (m, |
| | | 2H), 1.85-1.69 (m, 3H), 1.57-1.36 (m, 1H) |
| I- | 886.4 | 11.94 (br d, J = 8.38 Hz, 1 H) 11.00 (br s, 1 H) 8.06-8.15 (m, 1 H) 7.64-7.73 (m, 1 |
| 1380 | | H) 7.32 (t, J = 7.69 Hz, 1 H) 7.17 (d, J = 8.25 Hz, 1 H) 6.96 (d, J = 7.38 Hz, 1 H) 6.79- |
| | | 6.89 (m, 2 H) 6.66 (s, 1 H) 6.59 (dd, J = 8.44, 1.69 Hz, 1 H) 6.48 (br s, 1 H) 6.05-6.15 |
| | | (m, 1 H) 5.49 (br t, J = 5.38 Hz, 1 H) 5.12 (dd, J = 13.26, 5.00 Hz, 1 H) 4.57-4.67 (m, 2 |
| | | H) 4.10-4.38 (m, 4 H) 3.68-3.77 (m, 3 H) 3.54-3.67 (m, 2 H) 3.33-3.37 (m, 2 H) |
| | | 3.25 (br s, 4 H) 2.98-3.22 (m, 8 H) 2.88-2.98 (m, 1 H) 2.63 (br s, 7 H) 2.23-2.39 |
| | | (m, 3 H) 1.99-2.09 (m, 1 H) |

### Example 5 (Method 5): Synthesis of (S)-2-(5-(N-(2-(2-(2-(4-(4-(6-(1-(3-(1H-1,2,3-triazol-1-yl)propanoyl)-1,2,5,6-tetrahydropyridin-3-yl)-2-(dimethylcarbamoyl)-7-fluoro-LH-indol-4-yl)-3-methoxyphenyl)piperazin-1-yl)ethoxy)ethoxy)ethyl)-1-(isoquinolin-4-yl)piperidine-3-carboxamido)-2-oxopyridin-1 (2H)-yl)acetic acid (I-1072)

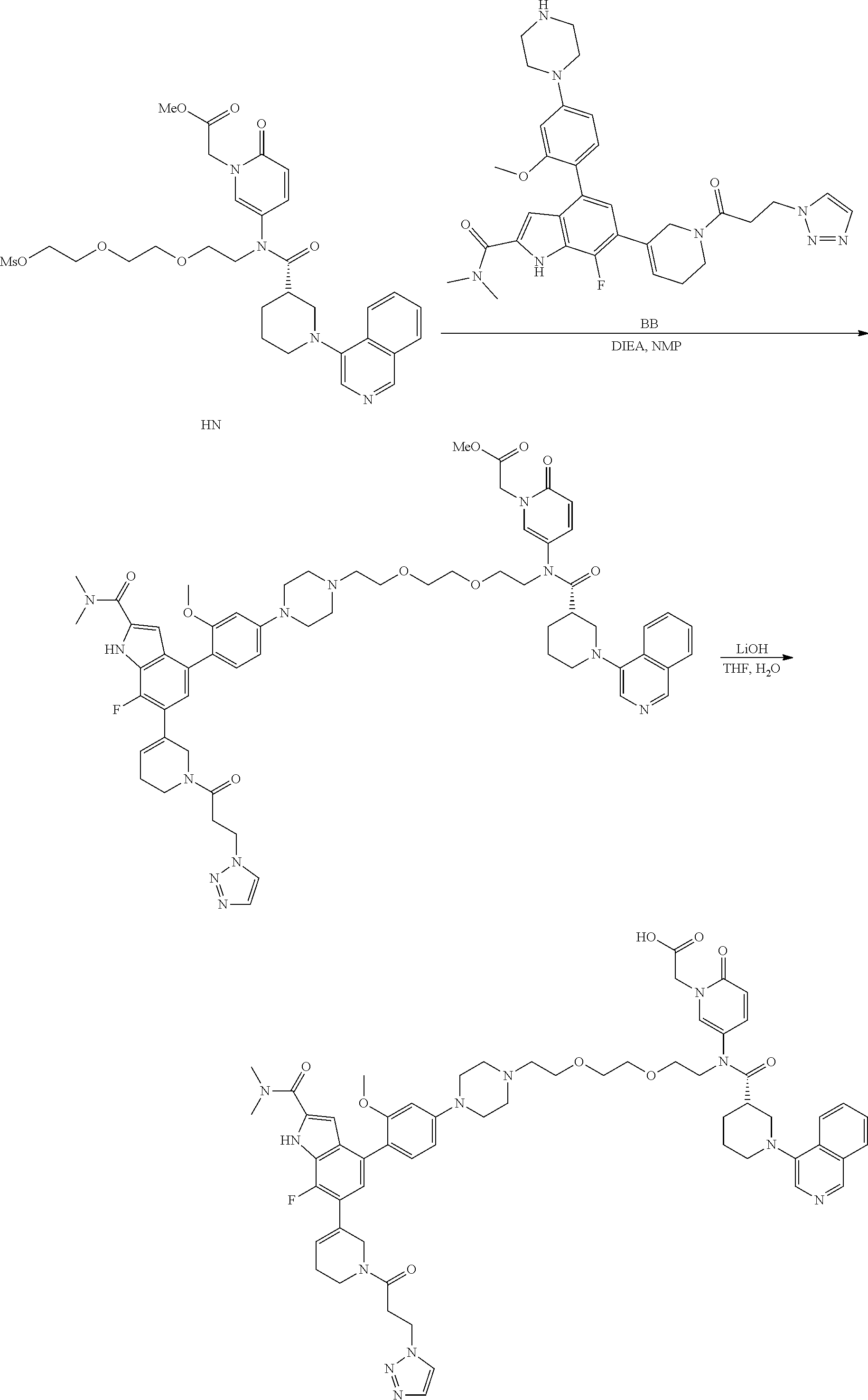

### Step 1—Methyl (S)-2-(5-(N-(2-(2-(2-(4-(4-(6-(1-(3-(1H-1,2,3-triazol-1-yl)propanoyl)-1,2,5,6-tetrahydropyridin-3-yl)-2-(dimethylcarbamoyl)-7-fluoro-1H-indol-4-yl)-3-methoxyphenyl)piperazin-1-yl)ethoxy)ethoxy)ethyl)-1-(isoquinolin-4-yl)piperidine-3-carboxamido)-2-oxopyridin-1 (2H)-yl)acetate

To a solution of methyl 2-[5-[[1-(4-isoquinolyl)piperidine-3-carbonyl]-[2-[2-(2-methylsulfonyloxyethoxy)ethoxy]ethyl]amino]-2-oxo-1-pyridyl]acetate (10 mg, 16 μmol, Intermediate HN) and 7-fluoro-4-(2-methoxy-4-piperazin-1-yl-phenyl)-N,N-dimethyl-6-[1-[3-(triazol-1-yl)propanoyl]-3,6-dihydro-2H-pyridin-5-yl]-1H-indole-2-carboxamide (9.52 mg, 15.7 μmol, Intermediate BB) in 1-methylpyrrolidin-2-one (1 mL) was added DIEA (10.3 mg, 79.3 μmol). The mixture was then stirred at 100° C. for 12 hrs. On completion, the reaction mixture was quenched by addition of H₂O (1 mL) at 25° C. The mixture was then purified by prep-HPLC (FA condition) to give the title compound (12 mg, 67% yield) as a yellow solid. LC-MS (ESI+) m/z 568.5 (M/2+H)⁺.

### Step 2—(S)-2-(5-(N-(2-(2-(2-(4-(4-(6-(1-(3-(TH-1,2,3-triazol-1-yl)propanoyl)-1,2,5,6-tetrahydropyridin-3-yl)-2-(dimethylcarbamoyl)-7-fluoro-1H-indol-4-yl)-3-methoxyphenyl)piperazin-1-yl)ethoxy)ethoxy)ethyl)-1-(isoquinolin-4-yl)piperidine-3-carboxamido)-2-oxopyridin-1 (2H)-yl)acetic acid

To a solution of methyl 2-[5-[2-[2-[2-[4-[4-[2-(dimethylcarbamoyl)-7-fluoro-6-[1-[3-(triazol-1-yl)propanoyl]-3,6-dihydro-2H-pyridin-5-yl]-1H-indol-4-yl]-3-methoxy-phenyl]piperazin-1-yl]ethoxy]ethoxy]ethyl-[I-(4-isoquinolyl)piperidine-3-carbonyl]amino]-2-oxo-1-pyridyl]acetate (10 mg, 8.81 μmol) in THE (0.2 mL) and MeOH (0.2 mL) and H₂O (0.2 mL) was added LiOH·H₂O (1.85 mg, 44.0 μmol). The mixture was stirred at 25° C. for 1 hr. On completion, the reaction mixture was concentrated under reduced pressure to remove solvent. The residue was purified by prep-HPLC (Hcolunn: Phenomenex luna C18 150*25 mm*10 um; mobile phase: [water (HCl)-ACN]; gradient:8%-38% B over 10 min) to give the title compound (2.5 mg. 25% yield) as a white solid. LC-MS (ESI⁺) m/z 1121.8 (M+H)⁺; ¹H NMR (400 MHz, DMSO-d₆) δ=11.97 (d, J=8.4 Hz, 1H), 9.34 (s, 1H), 8.36 (d, J=8.4 Hz, 1H), 8.17-8.09 (m, 2H), 7.96-7.88 (m, 3H), 7.71-7.66 (m, 1H), 7.59-7.49 (m, 1H), 7.30-7.25 (m, 1H), 7.23-7.20 (m, 1H), 7.24-7.20 (m, 1H), 7.15 (s, 1H), 7.02 (s, 1H), 6.88-6.80 (m, 1H), 6.73 (s, 1H), 6.67-6.62 (m, 1H), 6.57-6.49 (m, 1H), 6.45 (d, J=1.6 Hz, 1H), 6.13-6.07 (m, 1H), 4.66-4.56 (m, 4H), 4.35-4.29 (m, 2H), 3.82 (s, 2H), 3.72 (d, J=4.0 Hz, 4H), 3.65 (s, 2H), 3.57 (d, J=5.4 Hz, 4H), 3.51 (d, J=2.4 Hz, 4H), 3.11-3.09 (m, 4H), 2.95-2.88 (m, 4H), 2.85-2.79 (m, 2H), 2.68-2.66 (m, 1H), 2.55-2.53 (m, 4H), 2.34-2.32 (m, 2H), 2.29-2.22 (m, 2H), 2.00-1.83 (m, 2H), 1.82-1.72 (m, 2H), 1.72-1.53 (m, 3H), 1.27-1.18 (m, 2H).

TABLE 11
Compounds synthesized via Method 5, coupling the corresponding amines
and mesylates in Step 1, followed by hydrolysis in Step 2.

| | | | LCMS | |
|---|---|---|---|---|
| | | | (ESI+) | |
| | | | m/z | |
| I-#^{a} | Amine | Mesylate | (M + H)⁺ | ¹H NMR (400 MHz, DMSO-d₆) δ |
| I-1073 | BB | HT | 1209.5 | 12.04-11.91 (m, 1H), 11.06-10.41 (m, 1H), 9.55-9.39 |
| | | | | (m, 1H), 8.47 (br d, J = 7.2 Hz, 1H), 8.19 (s, 1H), 8.13- |
| | | | | 8.06 (m, 2H), 8.02-7.88 (m, 3H), 7.71-7.65 (m, 1H), |
| | | | | 7.62-7.46 (m, 1H), 7.21 (d, J = 8.4 Hz, 1H), 6.90-6.79 |
| | | | | (m, 1H), 6.72 (s, 1H), 6.66-6.60 (m, 1H), 6.53-6.43 (m, |
| | | | | 2H), 6.16-6.02 (m, 1H), 4.89-4.66 (m, 1H), 4.66-4.49 |
| | | | | (m, 4H), 4.36-4.29 (m, 2H), 3.97-3.89 (m, 2H), 3.85 (br |
| | | | | s, 3H), 3.71 (br d, J = 4.0 Hz, 4H), 3.64 (br s, 2H), 3.63- |
| | | | | 3.56 (m, 12H), 3.52 (br s, 6H), 3.27-3.19 (m, 6H), 3.09 |
| | | | | (td, J = 7.2, 10.4 Hz, 6H), 2.97-2.81 (m, 4H), 2.35-2.25 |
| | | | | (m, 2H), 1.96-1.86 (m, 1H), 1.83-1.76 (m, 1H), 1.72- |
| | | | | 1.55 (m, 2H) |
| I-1074 | BB | HQ | 1165.5 | 12.06-11.84 (m, 1H), 8.98 (s, 1H), 8.16-8.04 (m, 3H), |
| | | | | 7.88-7.76 (m, 2H), 7.75-7.63 (m, 3H), 7.59-7.46 (m, |
| | | | | 1H), 7.16 (d, J = 8.4 Hz, 1H), 6.93-6.80 (m, 1H), 6.65 (s, |
| | | | | 1H), 6.57 (br d, J = 8.4 Hz, 1H), 6.48 (br s, 2H), 6.16- |
| | | | | 6.06 (m. 1H), 4.71-4.55 (m, 3H), 4.42-4.24 (m, 3H), |
| | | | | 3.73-3.69 (m, 4H), 3.65-3.58 (m, 4H), 3.46 (br d. J = |
| | | | | 1.6 Hz, 8H), 3.28 (br s, 4H), 3.24 (br s, 7H), 3.13-3.06 |
| | | | | (m, SH), 3.04 (br s, 1H), 2.96-2.86 (m, 2H), 2.85-2.76 |
| | | | | (m, 2H), 2.68-2.58 (m, SH), 2.37-2.24 (m, 2H), 1.97- |
| | | | | 1.85 (m, 1H), 1.81-1,75 (m, 1H), 1.72-1.57 (m, 2H) |

^{a}The purification of the final compounds was achieved by reverse phase flash chromatography under a variety of conditions. Step 2 was run from 1-2 hrs at 25° C.

### Example 6 (Method 6): (S)-2-(5-(N-(8-(4-(4-(6-(l-(3-(1H-1,2,3-triazol-1-yl)propanoyl)-1,2,5,6-tetrahydropyridin-3-yl)-2-(dimethylcarbamoyl)-7-fluoro-LH-indol-4-yl)-3-methoxyphenyl)piperazin-1-yl)octyl)-1-(isoquinolin-4-yl)piperidine-3-carboxamido)-2-oxopyridin-1 (2H)-yl)acetic acid (I-1076)

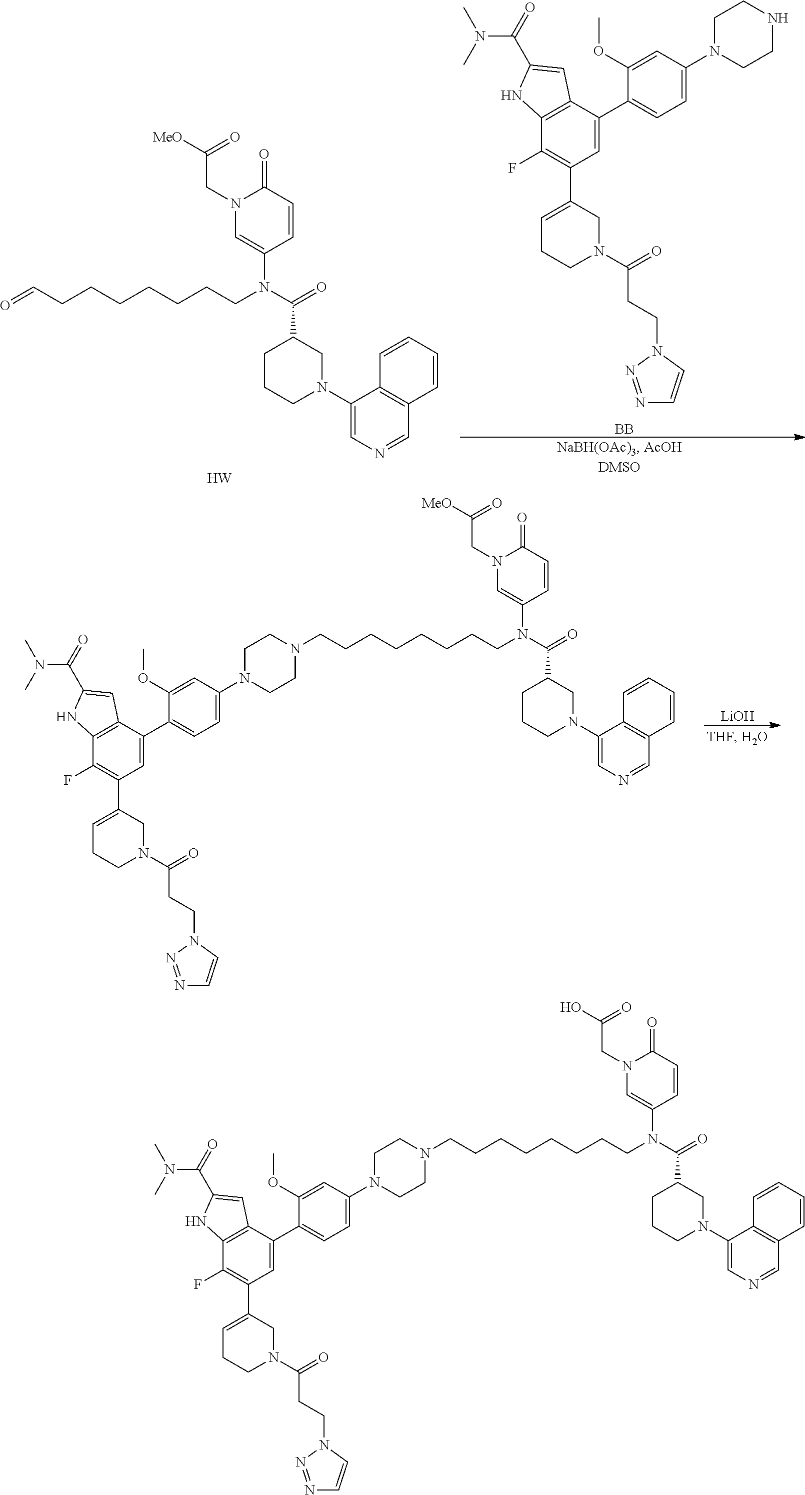

### Step 1—Methyl (S)-2-(5-(N-(8-(4-(4-(6-(1-(3-(1H-1,2,3-triazol-1-yl)propanoyl)-1,2,5,6-tetrahydropyridin-3-yl)-2-(dimethylcarbamoyl)-7-fluoro-1H-indol-4-yl)-3-methoxyphenyl)piperazin-1-yl)octyl)-1-(isoquinolin-4-yl)piperidine-3-carboxamido)-2-oxopyridin-1 (2H)-yl)acetate

A solution of methyl (S)-2-(5-(1-(isoquinolin-4-yl)-N-(8-oxooctyl)piperidine-3-carboxamido)-2-oxopyridin-1 (2H)-yl)acetate (150 mg, 219 μmol, Intermediate HW), 6-(1-(3-(1H-1,2,3-triazol-1-yl)propanoyl)-1,2,5,6-tetrahydropyridin-3-yl)-7-fluoro-4-(2-methoxy-4-(piperazin-1-yl)phenyl)-N,N-dimethyl-1H-indole-2-carboxamide (132 mg, 219 μmol, Intermediate BB), and NaBH(OAc)₃ (139 mg, 658 μmol) in DMSO (1 mL) and THF (1 mL) was stirred at 25° C. for 12 h. On completion, the reaction mixture was purified by reversed-phase HPLC (0.8 g/L ammonium bicarbonate) to give the title compound (70 mg, 26% yield) as a yellow solid. LC-MS (ESI⁺) m/z 1131.5 (M+H)⁺.

### Step 2—(S)-2-(5-(N-(8-(4-(4-(6-(1-(3-(1H-1,2,3-triazol-1-yl)propanoyl)-1,2,5,6-tetrahydropyridin-3-yl)-2-(dimethylcarbamoyl)-7-fluoro-1H-indol-4-yl)-3-methoxyphenyl)piperazin-1-yl)octyl)-1-(isoquinolin-4-yl)piperidine-3-carboxamido)-2-oxopyridin-1 (2H)-yl)acetic acid

A solution of methyl (S)-2-(5-(N-(8-(4-(4-(6-(1-(3-(1H-1,2,3-triazol-1-yl)propanoyl)-1,2,5,6-tetrahydropyridin-3-yl)-2-(dimethylcarbamoyl)-7-fluoro-1H-indol-4-yl)-3-methoxyphenyl)piperazin-1-yl)octyl)-1-(isoquinolin-4-yl)piperidine-3-carboxamido)-2-oxopyridin-1 (2H)-yl)acetate (70.0 mg, 61.8 μmol), and LiOH·H₂O (12.9 mg, 309 μmol) in THF (1 mL) and H₂O (1 mL) was stirred at 25° C. for 2 h. On completion, the crude residue was purified by reversed-phase HPLC (0.1% FA condition) to give the title compound (30 mg. FA) as a white solid. LC-MS (ESI⁺)/z 1117.9 (M+H)⁺; ¹H NMR (400 MHz, DMSO-d₆) δ=12.00-11.90 (m, 1H), 8.99 (s, 1H), 8.15-8.06 (m, 3H), 7.76 (d, J=8.8 Hz, 2H), 7.73-7.63 (m, 3H), 7.57-7.44 (m, 1H), 7.19 (d, J=8.4 Hz, 1H), 6.91-6.82 (m, 1H), 6.68 (s, 1H), 6.63-6.57 (m, 1H), 6.55-6.46 (m, 2H), 6.15-6.06 (m, 1H), 4.79-4.53 (m, 4H), 4.37-4.30 (m, 2H), 3.89-3.77 (m, 2H), 3.72 (d, J=4.0 Hz, 3H), 3.67-3.58 (m, 4H), 3.32 (s, 6H), 3.26 (d, J=12.4 Hz, 2H), 3.12-3.07 (m, 4H), 2.93-2.82 (m, 2H), 2.82-2.69 (m, 6H), 2.59-2.54 (m, 1H), 2.35-2.24 (m, 2H), 1.92-1.75 (m, 2H), 1.70-1.58 (m, 2H), 1.55-1.39 (m, 4H), 1.26 (s, 8H).

TABLE 12
Compounds synthesized via Method 6, the reductive amination of corresponding
amines and aldehydes in Step 1, followed by hydrolysis in Step 2.

| | | | LCMS | |
|---|---|---|---|---|
| | | | (ESI+) | |
| | | | m/z | |
| I-# | Amine | Aldehyde | (M + H)⁺ | ¹H NMR (400 MHz, DMSO-d₆) δ |
| I-1075 | BB | HZ | 1160.9 | 11.97-11.90 (m, 1H), 8.97 (s, 1H), 8.14-8.04 (m, 3H), |
| | | | | 7.83-7.74 (m, 2H), 7.73-7.62 (m, 3H), 7.54-7.42 (m, |
| | | | | 1H), 7.17 (d, J = 8.4 Hz, 1H), 6.90-6.80 (m, 1H), 6.65 (s, |
| | | | | 1H), 6.61-6.55 (m, 1H), 6.53-6.44 (m, 2H), 6.14-6.06 |
| | | | | (m, 1H), 4.66-4.57 (m, 3H), 4.42-4.24 (m, 3H), 3.70 (d, |
| | | | | J = 4.0 Hz, 4H), 3.67-3.55 (m, 6H), 3.13-3.03 (m, 7H), |
| | | | | 2.90-2.75 (m, 4H), 2.68-2.58 (m, 5H), 2.45-2.23 (m, |
| | | | | 5H), 1.93-1.75 (m, 2H), 1.66-1.38 (m, 6H), 1.23 (br s, |
| | | | | 16H) |
| I-1077 | BB | IC | 1201.7 | 11.96-11.91 (m, 1H), 8.97 (s, 1H), 8.12 (s, 1H), 8.09- |
| | | | | 8.05 (m, 1H), 7.82 (br s, 1H), 7.79-7.75 (m, 1H), 7.73- |
| | | | | 7.68 (m, 2H), 7.67-7.62 (m, 1H), 7.51-7.43 (m, 1H), |
| | | | | 7.24-7.13 (m, 2H), 6.96-6.80 (m, 2H), 6.64 (s, 1H), 6.57 |
| | | | | (br d, J = 7.2 Hz, 1H), 6.51-6.45 (m, 2H), 6.13-6.05 (m, |
| | | | | 1H), 4.66-4.57 (m, 3H), 4.35 (br s, 1H), 4.30 (br s, 1H), |
| | | | | 4.06 (t, J = 5.6 Hz, 1H), 3.70 (br d, J = 4.0 Hz, 3H), 3.59 |
| | | | | (br s, 3H), 3.23 (br s, 4H), 3.08 (br d, J = 7.2 Hz, 4H), 2.88 |
| | | | | (br d, J = 10.4 Hz, 2H), 2.83-2.77 (m, 2H), 2.71-2.65 (m, |
| | | | | 2H), 2.55 (br s, 4H), 2.38-2.31 (m, 4H), 2.26 (br d, J = 1.2 |
| | | | | Hz, 1H), 2.14-2.05 (m, 1H), 1.92-1.83 (m, 1H), 1.80- |
| | | | | 1.71 (m, 2H), 1.60-1.53 (m, 1H), 1.47 (br s, 2H), 1.39 (s, |
| | | | | 4H), 1.26 (br s, 4H), 1.22 (br s, 16H) |

^{a}The purification of the final compounds was achieved by reverse phase flash chromatography or prep-HPLC under a variety of conditions. Step 1 was run from 0-25° C. for 1-12 h. Step 2 was run from 1-2 h at 25° C. KOAc and 4 Å molecular sieves could also be added in Step 1 to promote the reaction, and THF could also be used as a co-solvent.

### Compounds Separated by SFC

### Example 7: Syntheses of (R)-3-((4-(4-(4-bromophenyl)piperidin-1-yl)-3-fluorophenyl)amino)piperidine-2,6-dione and (S)-3-((4-(4-(4-bromophenyl)piperidin-1-yl)-3-fluorophenyl)amino)piperidine-2,6-dione (used in the synthesis of (I-788) and (I-789))

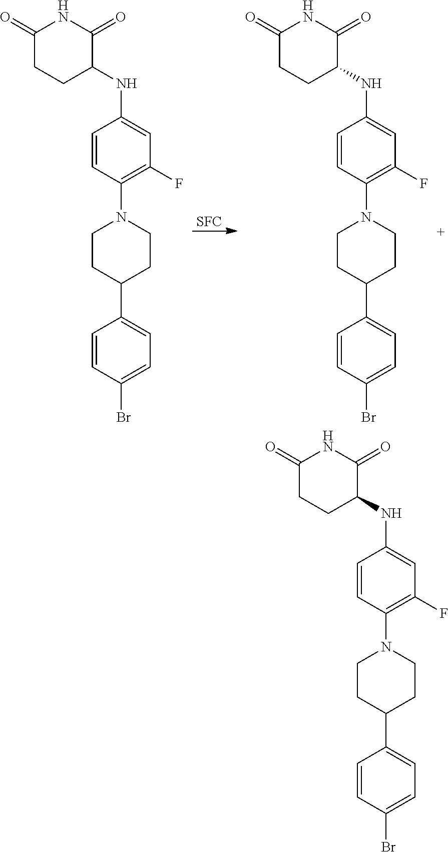

3-((4-(4-(4-bromophenyl)piperidin-1-yl)-3-fluorophenyl)amino)piperidine-2,6-dione (50 mg, 0.10 mmol) was separated by SFC (column: DAICEL CHIRALCEL OX (250 mm*30 mm, 10 um); mobile phase: [CO₂-i-PrOH/ACN]; B %: 60%, isocratic elution mode) to give:

First eluting isomer. (10 mg, 20% yield) as a white solid (LC-MS (ESI⁺) m/z 790.5 (M+H)⁺; ¹H NMR (400 MHz, DMSO-d6) 5=11.01-10.51 (m, 1H), 7.48 (d, J=8.4 Hz, 2H), 7.28 (d, J=8.4 Hz, 2H), 6.88 (t, J=9.2 Hz, 1H), 6.61-6.35 (m, 2H), 5.80 (d, J=7.6 Hz, 1H), 4.40-4.10 (m, 1H), 3.24 (d, J=11.6 Hz, 2H), 2.79-2.72 (m, 1H), 2.64 (s, 2H), 2.60 (d, J=2.8 Hz, 1H), 2.59-2.54 (m, 1H), 2.15-2.06 (m, 1H), 1.92-1.84 (m, 1H), 1.84-1.81 (m, 2H), 1.81-1.71 (m, 2H)); and

Second eluting isomer, (10 mg, 20% yield) as a white solid (LC-MS (ESI⁺) m/z 790.6 (M+H)⁺; ¹H NMR (400 MHz, DMSO-d6) 5=10.92-10.57 (m, 1H), 7.59-7.39 (m, 2H), 7.28 (d, J=8.4 Hz. 2H), 6.88 (t, J=9.2 Hz, 1H), 6.57-6.41 (m, 2H), 5.80 (d, J=7.6 Hz, 1H), 4.31-4.21 (m, 1H), 3.24 (d, J=11.6 Hz, 2H), 2.79-2.72 (m, 1H), 2.72-2.66 (m, 2H), 2.60 (s, 1H), 2.60-2.55 (m, 1H), 2.14-2.06 (m, 1H), 1.92-1.84 (m, 1H), 1.84-1.81 (m, 2H), 1.80-1.72 (m, 2H)). The absolute stereochemistry of the enantiomers was assigned arbitrarily.

### Examples I-790 and I-791: Syntheses of (R)-4-(4-(1-(1-(2,6-dioxopiperidin-3-yl)-3-methyl-2-oxo-2,3-dihydro-1H-benzo[d]imidazol-4-yl)piperidin-4-yl)-2-methylphenyl)-7-fluoro-6-(1-isobutyryl-1,2,5,6-tetrahydropyridin-3-yl)-N,N-dimethyl-1H-indole-2-carboxamide and (S)-4-(4-(1-(1-(2,6-dioxopiperidin-3-yl)-3-methyl-2-oxo-2,3-dihydro-1H-benzo[d]imidazol-4-yl)piperidin-4-yl)-2-methylphenyl)-7-fluoro-6-(1-isobutyryl-1,2,5,6-tetrahydropyridin-3-yl)-N,N-dimethyl-1H-indole-2-carboxamide

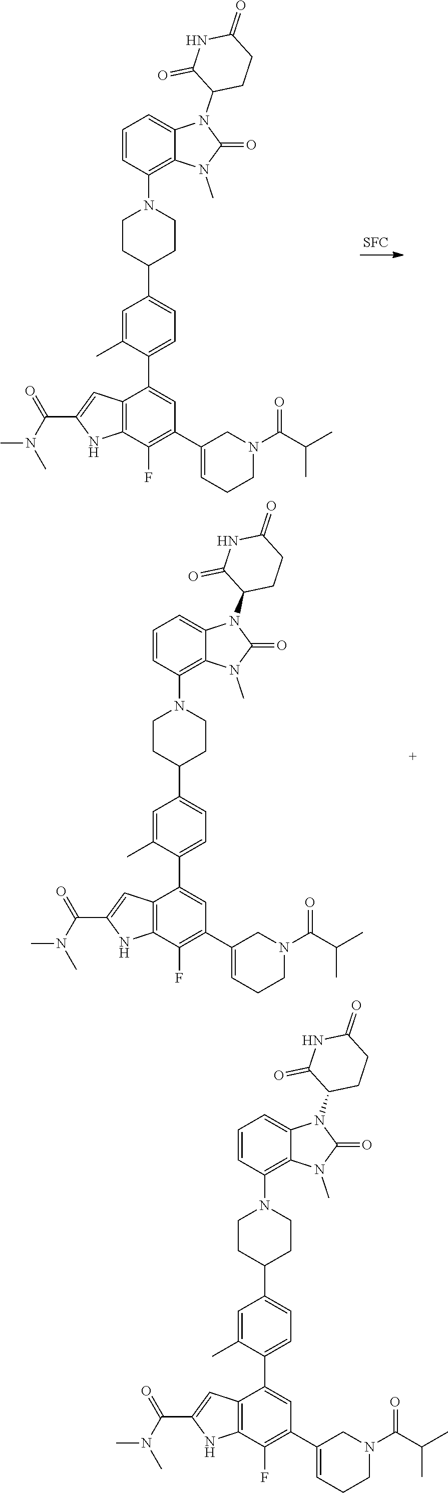

4-(4-(I-(1-(2,6-dioxopiperidin-3-yl)-3-methyl-2-oxo-2,3-dihydro-1H-benzo[d]imidazol-4-yl)piperidin-4-yl)-2-methylphenyl)-7-fluoro-6-(1-isobutyryl-1,2,5,6-tetrahydropyridin-3-yl)-N,N-dimethyl-1H-indole-2-carboxamide (110 mg, 139 μmol) was purified by SFC (column: DAICEL CHIRALPAK IA (250 mm*30 mm, 10 um); mobile phase: [Hexane-IPA]; B %:70%, isocratic elution mode) to give:

First eluting isomer, I-791 (30 mg, 27% yield) as a white solid (LC-MS (ESI⁺) m/z 788.5 (M+H)⁺; ¹H NMR (400 MHz, DMSO-d₆) δ=12.14 ((br s, 1H), 11.07 (br s, 1H), 7.31 (s, 1H), 7.28-7.20 (m, 2H), 7.06-6.96 (m, 2H), 6.93-6.81 (m, 2H), 6.40 (d, J=2.4 Hz, 1H), 6.15 (br s, 1H), 5.37 (br dd, J=5.2, 12.4 Hz, 1H), 4.51-4.26 (m, 2H), 3.71 (s, 3H), 3.69-3.62 (m, 2H), 3.26 (br d, J=11.2 Hz, 2H), 3.20-3.03 (m, 5H), 3.01-2.84 (m, 5H), 2.77-2.62 (m, 3H), 2.39-2.25 (m, 2H), 2.18 (br s, 3H), 2.06-1.92 (m, 5H), 1.08-0.98 (m, 6H)); and

Second eluting isomer, I-790 (32 mg, 29% yield) as a white solid (LC-MS (ESI⁺) m/z 788.3 (M+H)⁺; ¹H NMR (400 MHz, DMSO-d₆) δ=12.13 ((br s, 1H), 11.06 (br s, 1H), 7.31 (s, 1H), 7.28-7.19 (m, 2H), 7.06-6.96 (m, 2H), 6.93-6.80 (m, 2H), 6.40 (d, J=2.4 Hz, 1H), 6.15 (br s, 1H), 5.37 (br dd, J=5.6, 12.4 Hz, 1H), 4.55-4.25 (m, 2H), 3.71 (s, 3H), 3.69-3.63 (m, 2H), 3.26 (br d, J=11.2 Hz, 2H), 3.18-3.02 (m, 5H), 3.01-2.82 (m, 5H), 2.77-2.61 (m, 3H), 2.39-2.24 (m, 2H), 2.18 (br s, 3H), 2.05-1.93 (m, 5H), 1.09-0.95 (m, 6H)). The absolute stereochemistry of the enantiomers was assigned arbitrarily.

### Examples I-792 and I-793: Syntheses of (R)-6-(1-(3-(1H-pyrazol-1-yl)propanoyl)-1,2,5,6-tetrahydropyridin-3-yl)-4-(4-(1-(4-((2,6-dioxopiperidin-3-yl)amino)-2-fluoro-5-methoxyphenyl)piperidin-4-yl)-2-(trifluoromethoxy)phenyl)-7-fluoro-N,N-dimethyl-1H-indole-2-carboxamide and (S)-6-(1-(3-(1H-pyrazol-1-yl)propanoyl)-1,2,5,6-tetrahydropyridin-3-yl)-4-(4-(1-(4-((2,6-dioxopiperidin-3-yl)amino)-2-fluoro-5-methoxyphenyl)piperidin-4-yl)-2-(trifluoromethoxy)phenyl)-7-fluoro-N,N-dimethyl-1H-indole-2-carboxamide

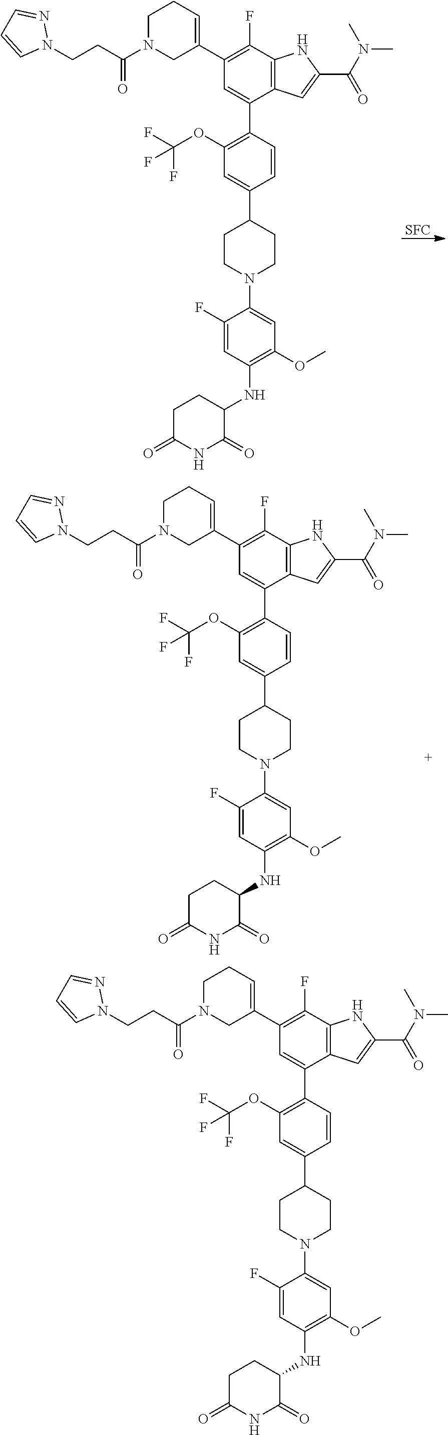

6-(1-(3-(1H-pyrazol-1-yl)propanoyl)-1,2,5,6-tetrahydropyridin-3-yl)-4-(4-(1-(4-((2,6-dioxopiperidin-3-yl)amino)-2-fluoro-5-methoxyphenyl)piperidin-4-yl)-2-(trifluoromethoxy)phenyl)-7-fluoro-N,N-dimethyl-1H-indole-2-carboxamide (60 mg) was separated by SFC (column: DAICEL CHIRALCEL OD (250 mm*30 mm, 10 um); mobile phase: [CO2-i-PrOH/ACN]; B %: 50%, isocratic elution mode) to give:

First eluting isomer, 1-793 (10.57 mg, 17.62% yield) as a white solid (LC-MS (ESI⁺) m/z 903.4 (M+H)⁺; ¹H NMR (400 MHz, DMSO-d6) δ=12.32-12.13 (m, 1H), 10.98-10.74 (m, 1H), 7.76-7.68 (m, 1H), 7.56 (d, J=8.0 Hz, 1H), 7.48 (d, J=8.1 Hz, 1H), 7.45-7.35 (m, 2H), 7.05-6.93 (m, 1H), 6.68 (d, J=8.1 Hz, 1H), 6.62-6.51 (m, 2H), 6.24-6.07 (m, 2H), 5.12 (d, J=6.5 Hz, 1H), 4.40-4.35 (m, 3H), 4.34-4.25 (m, 2H), 3.84 (s, 3H), 3.67-3.60 (m, 1H), 3.56 (t, J=5.8 Hz, 1H), 3.24 (d, J=2.3 Hz, 2H), 3.22-3.01 (m, 6H), 3.00-2.94 (m, 2H), 2.86-2.77 (m, 4H), 2.70-2.55 (m, 1H), 2.35-2.25 (m, 2H), 2.19-2.11 (m, 1H), 1.99-1.92 (m, 3H), 1.91-1.83 (m, 2H)) and

Second eluting isomer, I-792 (13.4 mg, 22% yield) as a white solid (LC-MS (ESI⁺) m/z 903.5 (M+H)⁺; ¹H NMR (400 MHz, DMSO-d6) δ=12.32-12.10 (m, 1H), 11.09-10.61 (m, 1H), 7.75-7.68 (m, 1H), 7.58 (d, J=8.0 Hz, 1H), 7.50-7.45 (m, 1H), 7.45-7.36 (m, 2H), 7.05-6.94 (m, 1H), 6.68 (d, J=8.0 Hz, 1H), 6.61-6.51 (m, 2H), 6.23-6.11 (m, 2H), 5.15-5.08 (m, 1H), 4.39-4.34 (m, 3H), 4.34-4.25 (m, 2H), 3.82 (s, 3H), 3.67-3.60 (m, 1H), 3.56 (t, J=5.6 Hz, 1H), 3.24 (s, 2H), 3.21-3.02 (m, 6H), 3.00-2.94 (m, 2H), 2.86-2.77 (m, 4H), 2.69-2.56 (m, 1H), 2.35-2.25 (m, 2H), 2.19-2.12 (m, 1H), 1.99-1.92 (m, 3H), 1.91-1.82 (m, 2H)). The absolute stereochemistry of the enantiomers was assigned arbitrarily.

### Examples I-794 and I-795: Syntheses of (R)-6-(1-(3-(1H-pyrazol-1-yl)propanoyl)-1,2,5,6-tetrahydropyridin-3-yl)-4-(4-(4-(4-((2,6-dioxopiperidin-3-yl)amino)-2-fluoro-5-methoxyphenyl)piperazin-1-yl)phenyl)-7-fluoro-N,N-dimethyl-1H-indole-2-carboxamide and (S)-6-(1-(3-(1H-pyrazol-1-yl)propanoyl)-1,2,5,6-tetrahydropyridin-3-yl)-4-(4-(4-(4-((2,6-dioxopiperidin-3-yl)amino)-2-fluoro-5-methoxyphenyl)piperazin-1-yl)phenyl)-7-fluoro-N,N-dimethyl-1H-indole-2-carboxamide

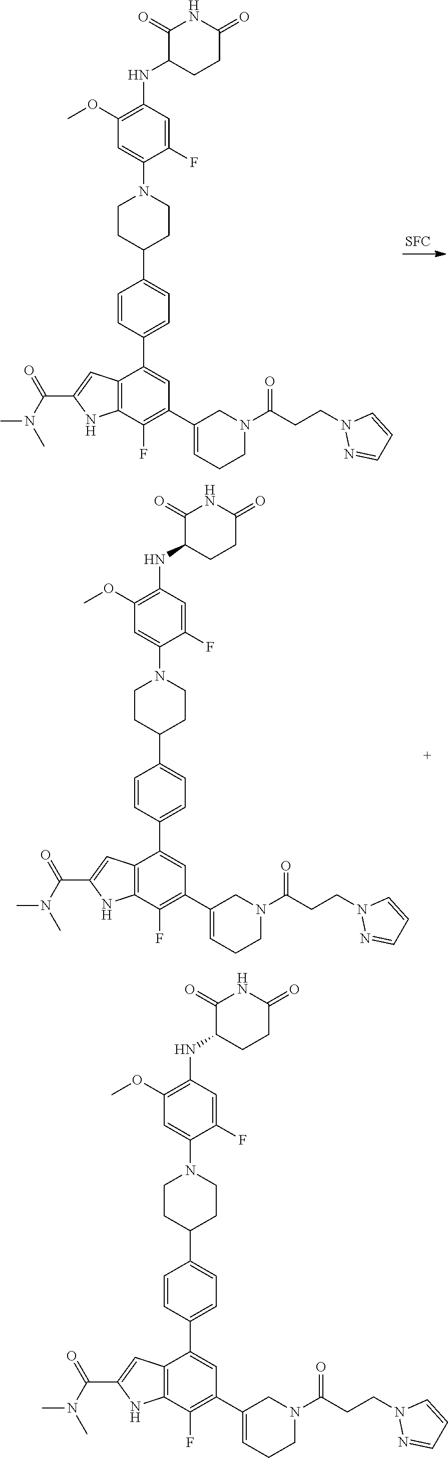

4-[4-[4-[4-[(2,6-dioxo-3-piperidyl)amino]-2-fluoro-5-methoxy-phenyl]piperazin-1-yl]phenyl]-7-fluoro-N,N-dimethyl-6-[1-(3-pyrazol-1-ylpropanoyl)-3,6-dihydro-2H-pyridin-5-yl]-1H-indole-2-carboxamide (75 mg, 91.47 μmol) was separated by SFC (column: DAICEL CHIRALCEL OX (250 mm*30 mm, 10 um):mobile phase: [HEXANE-IPA (0.1% IPAM)]:B %:100%, isocratic elution mode). Then each enantiomer was purified by reverse phase HPLC (0.1% FA condition) to give:

First eluting isomer, I-795 (20 mg, 26% yield) as white solid (LC-MS (ESI⁺) m/z 820.2 (M+H)⁺; ¹H NMR (400 MHz, DMSO-d₆) δ=12.12 (br d, J=7.2 Hz, 1H), 10.86 (s, 1H), 7.-5-7.70 (m, 1H), 7.56 (br d, J=8.4 Hz, 2H), 7.-5-7.37 (m, 1H), 7.12 (br d, J=8.8 Hz, 2H), 7.-2-6.93 (m, 1H), 6.88 (br s, 1H), 6.69 (d, J=8.0 Hz, 1H), 6.60 (d, J=14.4 Hz, 1H), 6.-4-6.11 (m, 2H), 5.-6-5.11 (m, 1H), 4.-0-4.26 (m, 5H), 3.83 (s, 3H), 3.66 (brt, J=5.6 Hz, 1H), 3.59 (brt, J=5.6 Hz, 1H), 3.36 (br d, J=4.8 Hz, 5H), 3.-2-3.06 (m, 9H), 3.-1-2.99 (m, 1H), 2.-8-2.95 (m, 1H), 2.-7-2.77 (m, 1H), 2.57 (br d, J=3.2 Hz, 1H), 2.34 (br s, 1H), 2.28 (br s, 1H), 2.-9-2.11 (m, 1H), 2.-0-1.88 (in, 1H)); and

Second eluting isomer, I-794 (10 mg, 13% yield) as white solid (LC-MS (ESI) m/z 820.7 (M+H)⁺: ¹H NMR (400 MHz, DMSO-d₆) δ=12.13 (br d, J=1.6 Hz, 1H), 10.85 (br s, 1H), 7.76-7.69 (m, 1H), 7.56 (br d, J=8.8 Hz, 2H), 7.46-7.36 (m, 1H), 7.12 (br d, J=8.8 Hz, 2H), 7.02-6.93 (m, 1H), 6.87 (br s, 1H), 6.69 (d, J=8.0 Hz, 1H), 6.60 (d, J=14.4 Hz, 1H), 6.24-6.11 (m, 2H), 5.14 (br d, J=6.8 Hz, 1H), 4.41-4.25 (m, 5H), 3.83 (s, 3H), 3.69-3.57 (m, 2H), 3.36 (br d. J=4.8 Hz, 5H), 3.24-3.05 (m, 9H), 3.02-2.97 (m, 2H), 2.87-2.77 (m, 1H), 2.57 (br d, J=3.2 Hz, 1H), 2.37-2.26 (m, 2H), 2.19-2.12 (m, 1H), 2.00-1.88 (m, 1H)). The absolute stereochemistry of the enantiomers was assigned arbitrarily.

### Examples 1-796 and I-797: Syntheses of (S)-6-(1-(3-(1H-pyrazol-1-yl)propanoyl)-1,2,5,6-tetrahydropyridin-3-yl)-4-(4-(1-(4-((2,6-dioxopiperidin-3-yl)amino)-2-fluorophenyl)piperidin-4-yl)phenyl)-7-fluoro-N,N-dimethyl-1H-indole-2-carboxamide and (R)-6-(1-(3-(1H-pyrazol-1-yl)propanoyl)-1,2,5,6-tetrahydropyridin-3-yl)-4-(4-(1-(4-((2,6-dioxopiperidin-3-yl)amino)-2-fluorophenyl)piperidin-4-yl)phenyl)-7-fluoro-N,N-dimethyl-1H-indole-2-carboxamide

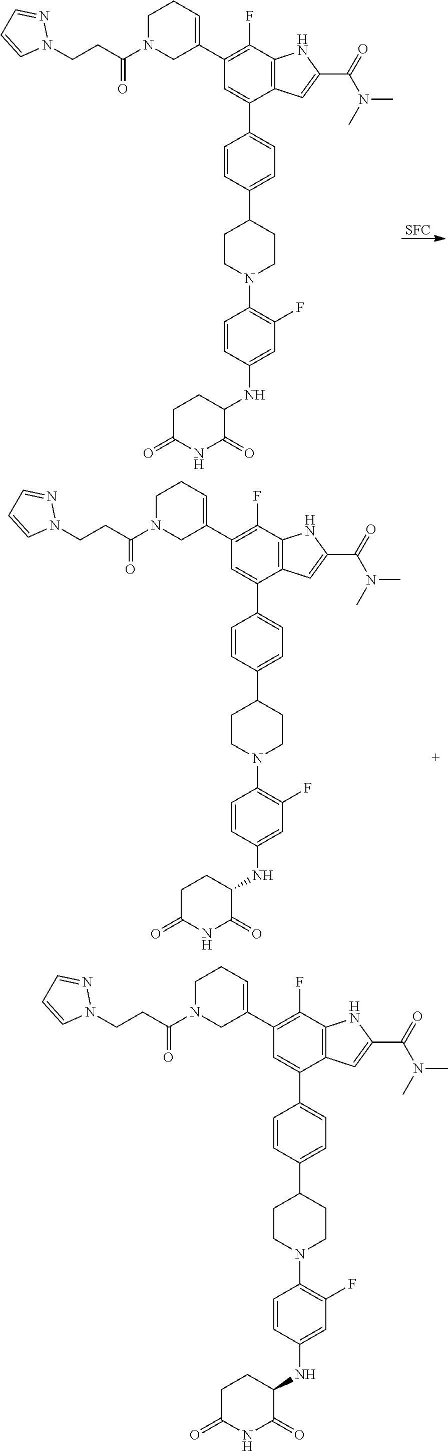

The 4-[4-[1-[4-[(2,6-dioxo-3-piperidyl)amino]-2-fluoro-phenyl]-4-piperidyl]phenyl]-7-fluoro-N,N-dimethyl-6-[1-(3-pyrazol-1-ylpropanoyl)-3,6-dihydro-2H-pyridin-5-yl]-1H-indole-2-carboxamide (0.6 g, 761 μmol) was purified by SFC (column: DAICEL CHIRALPAK AS (250 mm×30 mm, 10 um); mobile phase: [CO₂-i-PrOH/ACN]; B %:65%, isocratic elution mode) to give:

Second eluting isomer. I-796 (172 mg, 56% yield) as a white solid (LC-MS (ESI⁺) m/z 789.4 (M+H)⁺: ¹H NMR (400 MHz, DMSO-d6) δ=12.48-11.84 (m, 1H), 10.79 (s, 1H), 7.72 (d, J=11.6 Hz, 1H), 7.62 (d, J=7.6 Hz, 2H), 7.51-7.31 (m, 3H), 7.09-6.97 (m, 1H), 6.96-6.83 (m, 2H), 6.54 (d, J=14.8 Hz, 1H), 6.45 (d, J=9.2 Hz, 1H), 6.30-6.10 (m, 2H), 5.82 (d, J=7.2 Hz, 1H), 4.38 (s, 3H), 4.34-4.20 (m, 2H), 3.59 (s, 2H), 3.27 (d, J=11.2 Hz, 2H), 3.24-3.02 (m, 6H), 2.98 (d, J=7.2 Hz, 2H), 2.74 (d, J=1.6 Hz, 4H), 2.61 (s, 1H), 2.34 (d, J=0.8 Hz, 2H), 2.16-2.07 (m, 1H), 1.95-1.79 (m, 5H)); and

First eluting isomer, I-797 (129 mg, 41% yield) as a white solid (L C-MS (ESI⁺) m/z 789.4 (M+H)⁺; ¹H NMR (400 MHz, DMSO-d6) S=12.48-11.94 (m, 1H), 10.79 (d, J=2.8 Hz, 1H), 7.77-7.67 (m, 1H). 7.62 (d, J=7.6 Hz, 2H), 7.43 (d, J=7.2 Hz, 3H), 7.11-6.97 (m, 1H), 6.95-6.84 (m, 2H), 6.54 (d, J=15.6 Hz, 1H), 6.45 (d, J=8.4 Hz, 1H), 6.26-6.12 (m, 2H), 5.82 (d, J=7.6 Hz, 1H), 4.45-4.35 (m, 3H), 4.31 (s, 2H), 3.68-3.54 (m, 2H), 3.27 (d, J=10.4 Hz, 2H), 3.24-3.03 (m, 6H), 2.98 (d, J=7.2 Hz, 2H), 2.80-2.67 (m, 4H), 2.58 (d, J=17.6 Hz, 1H), 2.34 (s, 2H), 2.15-2.06 (m, 1H), 1.93-1.80 (m, 5H)). The absolute stereochemistry of the enantiomers was assigned arbitrarily.

### Example I-744 and I-745: (R)-6-(1-(3-(1H-pyrazol-1-yl)propanoyl)-1,2,5,6-tetrahydropyridin-3-yl)-4-(4-(4-(4-((2,6-dioxopiperidin-3-yl)amino)-2-fluoro-5-methoxyphenyl)piperazin-1-yl)-2,3-difluorophenyl)-7-fluoro-N,N-dimethyl-1H-indole-2-carboxamide (I-744) and (S)-6-(1-(3-(1H-pyrazol-1-yl)propanoyl)-1,2,5,6-tetrahydropyridin-3-yl)-4-(4-(4-(4-((2,6-dioxopiperidin-3-yl)amino)-2-fluoro-5-methoxyphenyl)piperazin-1-yl)-2,3-difluorophenyl)-7-fluoro-N,N-dimethyl-1H-indole-2-carboxamide (I-745)

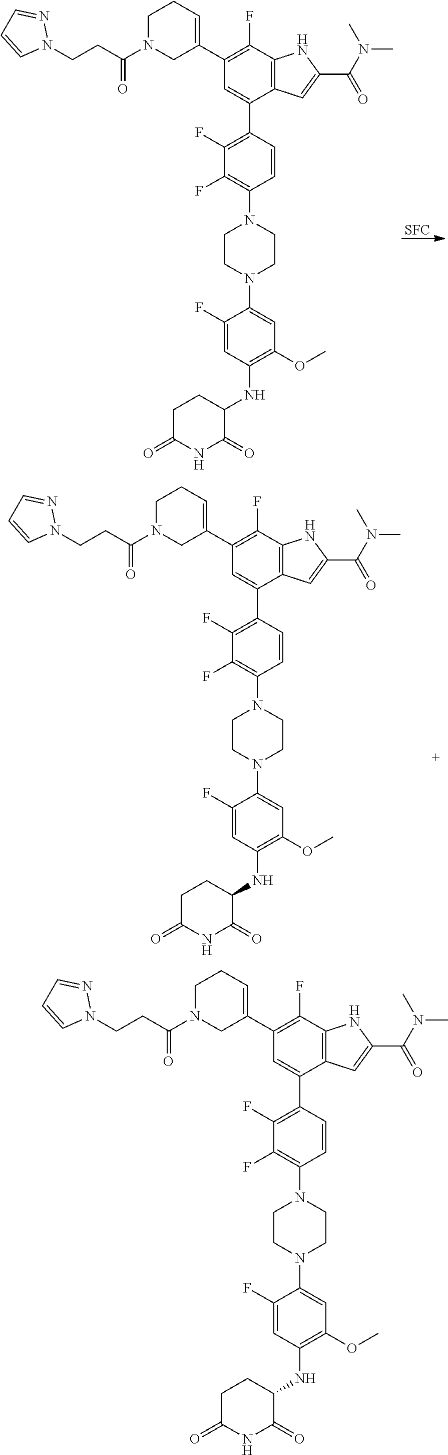

6-(1-(3-(1H-pyrazol-1-yl)propanoyl)-1,2,5,6-tetrahydropyridin-3-yl)-4-(4-(4-(4-((2,6-dioxopiperidin-3-yl)amino)-2-fluoro-5-methoxyphenyl)piperazin-1-yl)-2,3-difluorophenyl)-7-fluoro-N,N-dimethyl-1H-indole-2-carboxamide (2.5 g, 2.92 mmol, I-677) was separated by SFC (column: DAICEL CHIRALPAK IK (250 mm*50 mm, 10 um); mobile phase: [Hexane-IPA]; B %:100%, isocratic elution mode) and then purified by RPLC (0.1% FA condition) to give the first eluting isomer (R)-6-(1-(3-(1H-pyrazol-1-yl)propanoyl)-1,2,5,6-tetrahydropyridin-3-yl)-4-(4-(4-(4-((2,6-dioxopiperidin-3-yl)amino)-2-fluoro-5-methoxyphenyl)piperazin-1-yl)-2,3-difluorophenyl)-7-fluoro-N,N-dimethyl-1H-indole-2-carboxamide (I-744) (680 mg, 21% yield, FA, LC-MS (ESI⁺) m/z 856.5 (M+H)⁺; ¹H NMR (400 MHz, DMSO-d₆) δ=12.22 (s, 1H), 10.93-10.77 (m, 1H), 7.71 (dd, J=2.0, 11.2 Hz, 1H), 7.46-7.35 (m, 1H), 7.34-7.28 (m, 1H), 7.06-6.97 (m, 2H), 6.71 (d, J=8.0 Hz, 1H), 6.67 (d, J=2.0 Hz, 1H), 6.59 (d, J=14.4 Hz, 1H), 6.22-6.11 (m, 2H), 5.14 (d, J=6.8 Hz, 1H), 4.39-4.34 (m, 3H), 4.31-4.25 (m, 2H), 3.82 (s, 3H), 3.66-3.56 (m, 2H), 3.28 (s, 6H), 3.12 (s, 6H), 3.01-2.93 (m, 3H), 2.85-2.76 (m, 1H), 2.58-2.52 (m, 1H), 2.53 (d, J=6.0 Hz, 1H), 2.33 (s, 2H), 2.26 (d, J=2.0 Hz, 1H), 2.18-2.10 (m, 1H), 1.93 (dq, J=4.8, 12.8 Hz, 1H)) and the second eluting isomer (S)-6-(1-(3-(1H-pyrazol-1-yl)propanoyl)-1,2,5,6-tetrahydropyridin-3-yl)-4-(4-(4-(4-((2,6-dioxopiperidin-3-yl)amino)-2-fluoro-5-methoxyphenyl)piperazin-1-yl)-2,3-difluorophenyl)-7-fluoro-N,N-dimethyl-1H-indole-2-carboxamide (I-745) (501.5 mg, 20% yield, FA, LC-MS (ESI⁺) m/z 856.5 (M+H)⁺; ¹H NMR (400 MHz, DMSO-d₆) δ=12.26-12.17 (m, 1H), 10.85 (d, J=1.6 Hz, 1H), 7.74-7.69 (m, 1H), 7.44-7.36 (m, 1H), 7.34-7.29 (in, 1H), 7.05-6.98 (m, 2H), 6.71 (d, J=8.0 Hz, 1H), 6.67 (d, J=1.6 Hz, 1H), 6.59 (d, J=14.4 Hz, 1H), 6.21-6.13 (m, 2H), 5.14 (d, J=6.4 Hz, 1H), 4.38-4.35 (m, 3H), 4.31-4.25 (m, 2H), 3.82 (s, 3H), 3.66-3.56 (m, 2H), 3.28 (s, 6H), 3.12 (s, 6H), 3.01-2.94 (m, 3H), 2.85-2.77 (m, 1H), 2.55 (d, J=12.0 Hz, 1H), 2.33 (d, J=1.2 Hz, 2H), 2.27 (d, J=2.0 Hz, 1H), 2.17-2.10 (m, 1H), 1.98-1.89 (m, 1H)) as a white solids. The absolute stereochemistry of the enantiomers was assigned arbitrarily.

### Example I-742 and I-743: Syntheses of (S)-6-(1-(3-(1H-pyrazol-1-yl)propanoyl)-1,2,5,6-tetrahydropyridin-3-yl)-4-(4-(1-(4-(2,6-dioxopiperidin-3-yl)-2-fluorophenyl)piperidin-4-yl)phenyl)-7-fluoro-N,N-dimethyl-1H-indole-2-carboxamide (I-742) and (R)-6-(1-(3-(1H-pyrazol-1-yl)propanoyl)-1,2,5,6-tetrahydropyridin-3-yl)-4-(4-(1-(4-(2,6-dioxopiperidin-3-yl)-2-fluorophenyl)piperidin-4-yl)phenyl)-7-fluoro-N,N-dimethyl-1H-indole-2-carboxamide (I-743)

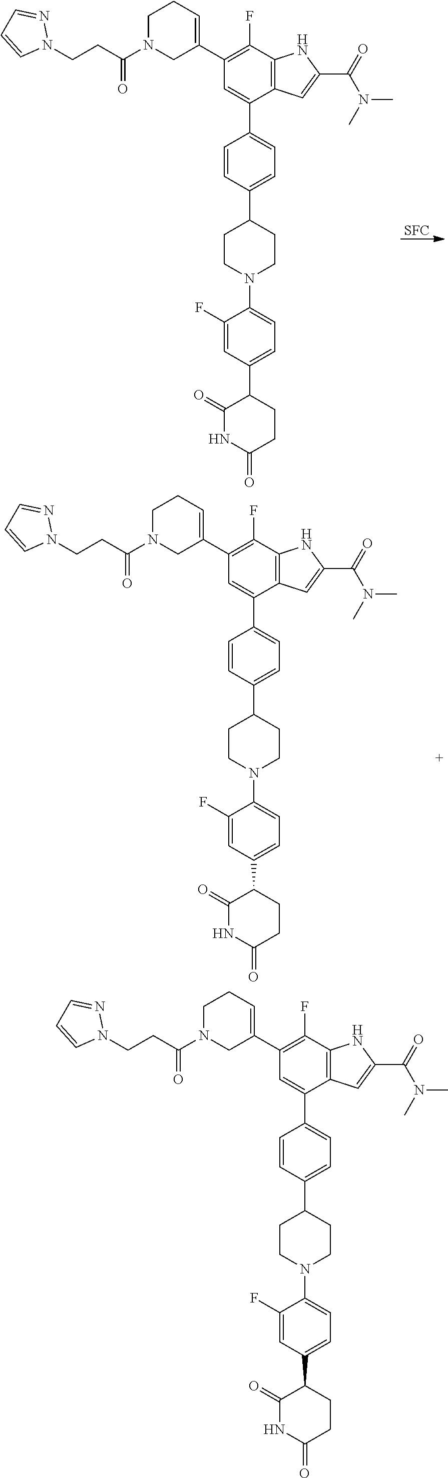

6-(1-(3-(1H-pyrazol-1-yl)propanoyl)-1,2,5,6-tetrahydropyridin-3-yl)-4-(4-(I-(4-(2,6-dioxopiperidin-3-yl)-2-fluorophenyl)piperidin-4-yl)phenyl)-7-fluoro-N,N-dimethyl-1H-indole-2-carboxamide (1.1 g, 1.42 mmol, I-628) was separated by SFC (column: DAICEL CHIRALPAK AS (250 mm*30 mm, 10 um); mobile phase: [CO₂-i-PrOH/ACN]; B %:70%, isocratic elution mode) to give the first eluting peak (S)-6-(1-(3-(1H-pyrazol-1-yl)propanoyl)-1,2,5,6-tetrahydropyridin-3-yl)-4-(4-(1-(4-(2,6-dioxopiperidin-3-yl)-2-fluorophenyl)piperidin-4-yl)phenyl)-7-fluoro-N,N-dimethyl-1H-indole-2-carboxamide (1-742) (310 mg, LC-MS (ESI⁺) m/z 774.4 (M+H); ¹H NMR (400 MHz, DMSO-d₆) δ=12.25-12.14 (m, 1H), 10.83 (s, 1H), 7.73 (dd, J=1.6, 11.2 Hz, 1H), 7.63 (d, J=7.6 Hz, 2H), 7.48-7.36 (m, 3H), 7.09-6.97 (m, 4H), 6.88 (s, 1H), 6.23-6.14 (m, 2H), 4.41-4.31 (m, 4H), 3.83 (dd, J=4.8, 11.6 Hz, 1H), 3.68-3.57 (m, 2H), 3.50 (d, J=11.2 Hz, 2H), 3.28-3.05 (m, 6H), 3.03-2.94 (m, 3H), 2.82 (t, J=10.8 Hz, 2H), 2.76-2.70 (m, 1H), 2.69-2.62 (m, 1H), 2.34 (s, 1H), 2.30-2.18 (m, 2H), 2.02 (td, J=4.4, 8.4 Hz, 1H), 1.98-1.86 (m, 4H)) and the second eluting peak (R)-6-(1-(3-(1H-pyrazol-1-yl)propanoyl)-1,2,5,6-tetrahydropyridin-3-yl)-4-(4-(1-(4-(2,6-dioxopiperidin-3-yl)-2-fluorophenyl)piperidin-4-yl)phenyl)-7-fluoro-N,N-dimethyl-1H-indole-2-carboxamide (I-743) (220 mg; LC-MS (ESI⁺) m/z 774.2 (M+H); H NMR (400 MHz, DMSO-d₆) δ=12.26-12.09 (m, 1H), 10.82 (s, 1H), 7.72 (dd, J=1.6, 11.2 Hz, 1H), 7.62 (br d, J=7.6 Hz, 2H), 7.47-7.36 (m, 3H), 7.09-6.96 (m, 4H), 6.87 (s, 1H), 6.23-6.12 (m, 2H), 4.42-4.28 (m, 4H), 3.82 (dd, J=4.8, 11.6 Hz, 1H), 3.68-3.56 (m, 2H), 3.49 (d, J=11.2 Hz, 2H), 3.27-3.04 (m, 6H), 2.97 (q. J=7.6 Hz, 3H), 2.82 (t, J=10.8 Hz, 2H), 2.77-2.69 (m, 1H), 2.68-2.62 (m, 1H), 2.33 (s, 1H), 2.30-2.15 (m, 2H), 2.02 (td, J=4.4, 8.8 Hz, 1H), 1.97-1.84 (m, 4H)) as a white solids. The absolute stereochemistry of the enantiomers was assigned arbitrarily.

### Examples I-1078 & 1-1079: Synthesis of (S)-6-(1-(3-(1H-pyrazol-1-yl)propanoyl)-1,2,5,6-tetrahydropyridin-3-yl)-4-(2-chloro-4-(4-(2-(1-(2,6-dioxopiperidin-3-yl)-3-methyl-1H-indazol-4-yl)ethyl)piperazin-1-yl)-5-fluorophenyl)-7-fluoro-N,N-dimethyl-1H-indole-2-carboxamide (I-1078) and (R)-6-(1-(3-(1H-pyrazol-1-yl)propanoyl)-1,2,5,6-tetrahydropyridin-3-yl)-4-(2-chloro-4-(4-(2-(1-(2,6-dioxopiperidin-3-yl)-3-methyl-1H-indazol-4-yl)ethyl)piperazin-1-yl)-5-fluorophenyl)-7-fluoro-N,N-dimethyl-1H-indole-2-carboxamide (I-1079)

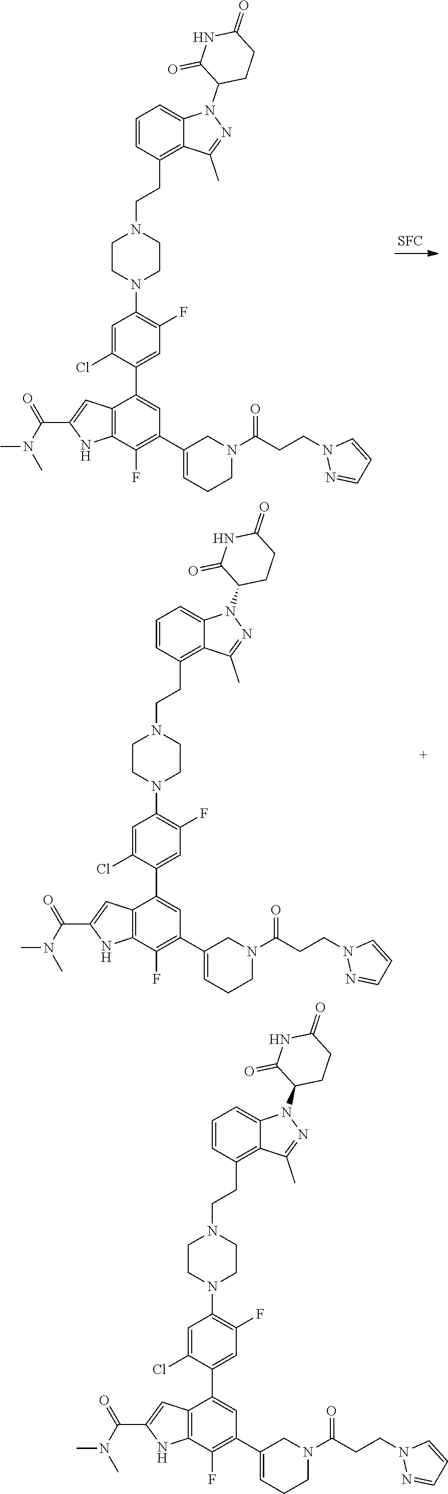 [SMILES: Cc1nn(C2CCC(=O)NC2=O)c2cccc(CCN3CCN(c4cc(Cl)c(-c5cc(C6=CCCN(C(=O)CCn7cccn7)C6)c(F)c6[nH]c(C(=O)N(C)C)cc56)cc4F)CC3)c12 ; "SFC" ; Cc1nn([C@H]2CCC(=O)NC2=O)c2cccc(CCN3CCN(c4cc(Cl)c(-c5cc(C6=CCCN(C(=O)CCn7cccn7)C6)c(F)c6[nH]c(C(=O)N(C)C)cc56)cc4F)CC3)c12 ; Cc1nn([C@@H]2CCC(=O)NC2=O)c2cccc(CCN3CCN(c4cc(Cl)c(-c5cc(C6=CCCN(C(=O)CCn7cccn7)C6)c(F)c6[nH]c(C(=O)N(C)C)cc56)cc4F)CC3)c12]

To a solution of 6-(1-(3-(1H-pyrazol-1-yl)propanoyl)-1,2,5,6-tetrahydropyridin-3-yl)-4-(2-chloro-4-(4-(2-(1-(2,6-dioxopiperidin-3-yl)-3-methyl-1H-indazol-4-yl)ethyl)piperazin-1-yl)-5-fluorophenyl)-7-fluoro-N,N-dimethyl-1H-indole-2-carboxamide (1 g, I-931) was purified by reversed-phase HPLC (column: REGIS(S,S)WHELK-O1 (250 mm*25 mm, 10 um); mobile phase: [CO2-i-PrOH/ACN]:B %:80%, isocratic elution mode) to give (S)-6-(1-(3-(1H-pyrazol-1-yl)propanoyl)-1,2,5,6-tetrahydropyridin-3-yl)-4-(2-chloro-4-(4-(2-(1-(2,6-dioxopiperidin-3-yl)-3-methyl-1H-indazol-4-yl)ethyl)piperazin-1-yl)-5-fluorophenyl)-7-fluoro-N,N-dimethyl-1H-indole-2-carboxamide (80 mg, 80% yield) as a white solid (LC-MS (ESI⁺) m/z 891.3 (M+H)⁺; ¹H NMR (400 MHz, DMSO-d₆) δ=12.24-12.13 (m, 1H), 11.11-11.01 (m, 1H), 7.79-7.65 (m, 1H), 7.49-7.36 (m, 2H), 7.35-7.27 (m, 2H), 7.22-7.16 (m, 1H), 6.99-6.96 (m, 1H), 6.57-6.44 (m, 1H), 6.29-6.05 (m, 2H), 5.77-5.67 (m, 1H), 4.39-4.29 (m, 4H), 3.66-3.56 (m, 2H), 3.26-3.11 (m, 10H), 3.04 (br s, 4H), 2.75-2.66 (m, 12H), 2.33 (br s, 4H)) and (R)-6-(1-(3-(1H-pyrazol-1-yl)propanoyl)-1,2,5,6-tetrahydropyridin-3-yl)-4-(2-chloro-4-(4-(2-(1-(2,6-dioxopiperidin-3-yl)-3-methyl-1H-indazol-4-yl)ethyl)piperazin-1-yl)-5-fluorophenyl)-7-fluoro-N,N-dimethyl-1H-indole-2-carboxamide (72 mg. 72% yield) as a white solid (LC-MS (ESI⁺) m/z 891.3 (M+H)⁺; ¹H NMR (400 MHz. DMSO-d₆) δ=12.24-12.13 (m, 1H), 11.11-11.01 (m, 1H), 7.79-7.65 (m, 1H), 7.49-7.36 (m, 2H), 7.35-7.27 (m, 2H), 7.22-7.16 (m, 1H), 6.99-6.96 (m, 1H), 6.57-6.44 (m, 1H), 6.29-6.05 (m, 2H), 5.77-5.67 (m, 1H), 4.39-4.29 (m, 4H), 3.66-3.56 (m, 2H), 3.26-3.11 (m, 10H), 3.04 (br s, 4H), 2.75-2.66 (m, 12H), 2.33 (br s, 4H)). The absolute stereochemistry of the enantiomers was assigned arbitrarily.

### (S)-6-(1-(3-(1H-1,2,3-triazol-1-yl)propanoyl)-1,2,5,6-tetrahydropyridin-3-yl)-4-(4-(1-(4-(2,6-dioxopiperidin-3-yl)-2-fluorophenyl)piperidin-4-yl)phenyl)-7-fluoro-N,N-dimethyl-1H-indole-2-carboxamide (I-1438) and (R)-6-(1-(3-(1H-1,2,3-triazol-1-yl)propanoyl)-1,2,5,6-tetrahydropyridin-3-yl)-4-(4-(1-(4-(2,6-dioxopiperidin-3-yl)-2-fluorophenyl)piperidin-4-yl)phenyl)-7-fluoro-N,N-dimethyl-1H-indole-2-carboxamide (I-1439)

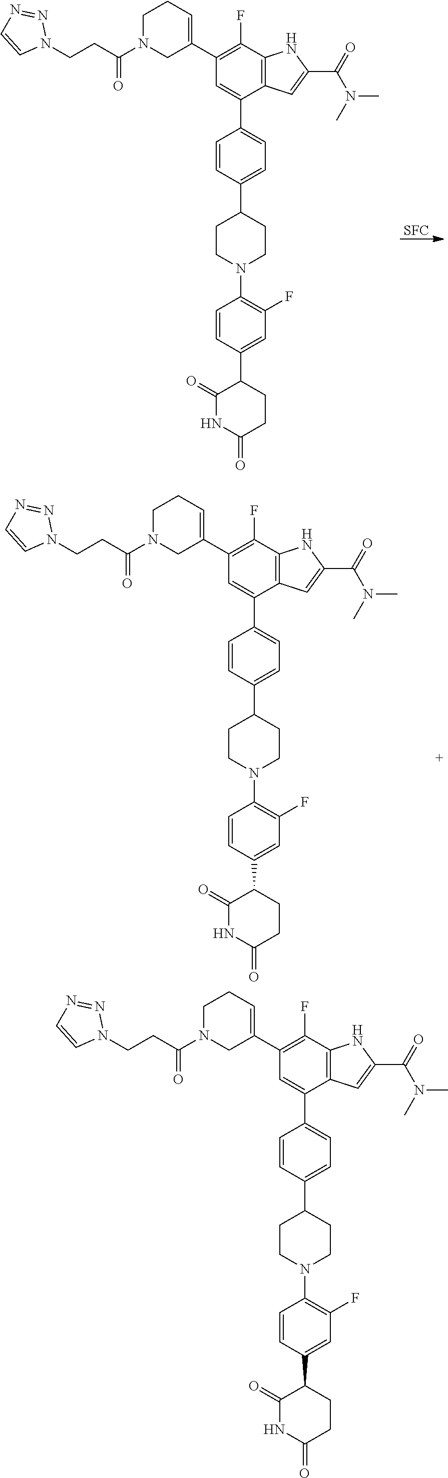

6-(1-(3-(1H-1,2,3-triazol-1-yl)propanoyl)-1,2,5,6-tetrahydropyridin-3-yl)-4-(4-(1-(4-(2,6-dioxopiperidin-3-yl)-2-fluorophenyl)piperidin-4-yl)phenyl)-7-fluoro-N,N-dimethyl-1H-indole-2-carboxamide (3.5 g, I-1015) was separated by SFC separation (column: REGIS (s,s) WHELK-01 (250 mm*50 mm, 10 um); mobile phase: [Hexane-EtOH/CAN (4:1)]; B %:95%, isocratic elution mode) to give the two isomers. The first fraction was collected as (S)-6-(1-(3-(1H-1,2,3-triazol-1-yl)propanoyl)-1,2,5,6-tetrahydropyridin-3-yl)-4-(4-(1-(4-(2,6-dioxopiperidin-3-yl)-2-fluorophenyl)piperidin-4-yl)phenyl)-7-fluoro-N,N-dimethyl-1H-indole-2-carboxamide (1.4 g, 35% yield (¹H NMR (400 MHz, DMSO-d₆) δ 12.18 (s, 1H), 10.83 (s, 1H), 8.14-8.09 (m, 1H), 7.72-7.67 (m, 1H), 7.63 (d, J=8.0 Hz, 2H), 7.44 (d, J=8.4 Hz, 2H), 7.10-6.97 (m, 4H), 6.88 (d, J=2.8 Hz, 1H), 6.17 (s, 1H), 4.69-4.59 (m, 2H), 4.43-4.31 (m, 2H), 3.83 (dd, J=4.8, 11.6 Hz. H), 3.70-3.58 (s, 2H), 3.50 (d, J=11.6 Hz, 2H), 3.27-2.99 (m, 8H), 2.82 (t 9 J=10.8 Hz, 2H), 2.77-2.62 (m, 2H), 2.53 (s, 1H), 239-2.32 (m, 1H), 2.30-2.26 (H, 1H), 2.24-2.15 (m, 1H), 2.09-2.02 (m, 1H), 1.98-1.83 (m, 4H); LC-MS (ESI⁺) m/z 775.3 (M (H)). The second fraction was collected as (R)-6-(1-(3-(1H-1,2,3-triazol-1-yl)propanoyl)-1,2,5,6-tetrahydropyridin-3-yl)-4-(4-(1-(4-(2,6-dioxopiperidin-3-yl)-2-fluorophenyl)piperidin-4-yl)phenyl)-7-fluoro-N,N-dimethyl-1H-indole-2-carboxamide (1.4 g, 352 yield) (H NMR (400 MHz, DMSO-d₆) 12.19 (d, J=7.2 Hz, 2H), 10.83 (s, 1H), 8.14-8.08 (m, 1H), 7.73-7.67 (m, 1H), 7.65-7.60 (m, 2H), 7.44 (d, J=8.0 Hz, 2H), 7.11-6.96 (m, 4H), 6.88 (s, 1H), 6.17 (d, J=1.6 Hz, 1H), 4.68-4.60 (m, 2H), 4.42-4.33 (m, 2H), 3.82 (dd, J=4.8, 11.6 Hz, 1H), 3.67 (t, J=5.6 Hz, H), 3.61 (t, J=5.6 Hz, 1H), 3.50 (d, J=11.6 Hz, 2H), 3.26 (3.02 (m, 8H), 2.82 (t, J=10.8 Hz, 2H), 2.73 (td, J=4.4, 15.2 Hz, 1H), 2.69-2.62 (m, 1H), 2.53 (s, 1H), 2.37 (s, 1H), 2.31-2.25 (m, 1H), 2.24-2.16 (m, 1H), 2.06-1.99 (m, 4H), 1.98-1.84 (m, 4H); LC-MS (ESI⁺) m/z 775.3 (M+H)⁺). The absolute stereochemistry of the enantiomers was assigned arbitrarily.

TABLE 13
Compounds synthesized via further methodology

| | LCMS | |
|---|---|---|
| | (ESI+) | |
| | m/z | |
| I-# | (M + H)⁺ | ¹H NMR (400 MHz, DMSO-d₆) δ |
| I-1 | 852.9 | 11.77-11.53 (m, 1H), 11.08 (s, 1H), 8.18 (s, 1H), 8.14-8.07 (m, 1H), 7.74-7.65 (m, |
| | | 1H), 7.56 (d, J = 8.8 Hz, 2H), 7.44-7.29 (m, 1H), 7.22-7.12 (m, 1H), 7.12-6.96 |
| | | (m, 4H), 6.94-6.81 (m, 2H), 6.32 (s, 1H), 5.34 (dd, J = 5.2, 12.6 Hz, 1H), 4.73-4.55 |
| | | (m, 2H), 4.49-4.32 (m, 2H), 3.65-3.56 (m, 2H), 3.34 (s, 3H), 3.23 (s, 6H), 3.17- |
| | | 3.09 (m, 4H), 2.95-2.71 (m, 2H), 2.71-2.58 (m, 4H), 2.54 (d, J = 4.4 Hz, 4H), |
| | | 2.47-2.32 (m, 4H), 2.32-2.19 (m, 1H), 2.09-1.93 (m, 1H), 1.89-1.70 (m, 2H) |
| I-2 | 852.9 | 11.67 (s, 1H), 11.09 (br s, 1H), 8.34 (br s, 1H), 8.14-8.11 (m, 1H), 7.72-7.69 (m, |
| | | 1H), 7.57 (br d, J = 8.4 Hz, 2H), 7.38-7.34 (m, 1H), 7.18-7.14 (m, 1H), 7.07 (br d, |
| | | J = 8.8 Hz, 2H), 6.99-6.97 (m, 2H), 6.94-6.91 (m, 1H), 6.87 (br s, 1H), 6.33 (br s, |
| | | 1H), 5.40-5.34 (m, 1H), 4.67-4.63 (m, 2H), 4.45-4.38 (m, 2H), 3.64 (br s, 2H), |
| | | 3.60 (s, 3H), 3.22-3.21 (m, 3H), 3.16 (br s, 2H), 3.12-3.10 (m, 2H), 3.03-2.94 (m, |
| | | 4H), 2.92-2.83 (m, 2H), 2.75-2.60 (m, 4H), 2.57-2.55 (m, 3H), 2.45-2.43 (m, |
| | | 2H), 2.33 (br s, 2H), 1.99 (br dd, J = 4.8, 10.4 Hz, 2H), 1.83 (br s, 2H) |
| I- | 417.0b | 11.77-11.51 (m, 1H), 11.08 (s, 1H), 9.52 (d, J = 3.6 Hz, 1H), 8.16-8.08 (m, 1H), |
| 104 | | 7.74-7.66 (m, 1H), 7.15-7.06 (m, 1H), 7.05-6.96 (m, 2H), 6.90-6.79 (m, 2H), |
| | | 6.63-6.53 (m, 1H), 6.27 (s, 1H), 5.34 (dd, J = 5.2, 12.8 Hz, 1H), 4.64 (t, J = 6.8 Hz, |
| | | 2H), 4.46-4.20 (m, 2H), 3.78 (d, J = 12.4 Hz, 2H), 3.63-3.56 (m, 4H), 3.33 (s, 6H), |
| | | 3.19 (s, 4H), 3.14-2.99 (m, 6H), 2.69 (d, J = 12.8 Hz, 3H), 2.66-2.59 (m, 3H), |
| | | 2.39-2.20 (m, 2H), 2.07-1.94 (m, 1H), 1.74-1.55 (m, 4H), 1.35 (s, 6H) |
| I- | 843.3 | 11.09-11.02 (m, 1H), 8.12 (s, 1H), 7.82-7.68 (m, 2H), 7.52 (s, 1H), 7.42 (d, J = 7.6 |
| 119 | | Hz, 1H), 6.91 (d, J = 8.8 Hz, 1H), 6.73 (d, J = 2.0 Hz, 1H), 6.56 (dd, J = 2.0, 8.8 Hz, |
| | | 1H), 6.38-6.30 (m, 1H), 5.32-5.24 (m, 1H), 4.64 (t, J = 6.8 Hz, 2H), 4.56-4.50 (m, |
| | | 1H), 4.37 (d, J = 8.0 Hz, 2H), 4.07-3.95 (m, 1H), 3.87-3.77 (m, 1H), 3.59 (td, J = |
| | | 5.6, 17.2 Hz, 2H), 3.19-3.13 (m, 6H), 3.08-3.01 (m, 4H), 2.94-2.84 (m, 2H), 2.78 |
| I- | 698.4 | 11.98-11.85 (m, 1H), 11.29-10.99 (m, 1H), 8.43 (s, 1H), 8.13 (s, 1H), 7.70 (d, J = |
| 144 | | 3.2 Hz, 1H), 7.55-7.48 (m, 2H), 7.42-7.33 (m, 1H), 7.32-6.98 (m, 2H), 6.92-6.84 |
| | | (m, 1H), 6.34 (br d, J = 3.2 Hz, 1H), 5.43 (dd, J = 5.2, 12.8 Hz, 1H), 4.64 (br t, J = 6.8 |
| | | Hz, 2H), 4.43-4.33 (m, 2H), 3.66-3.54 (m, 2H), 3.37 (s, 3H), 3.30 (br s, 4H), 3.19- |
| | | 3.07 (m, 4H), 2.68-2.59 (m, 2H), 2.33 (br d, J = 1.6 Hz, 2H), 2.30-2.21 (m, 1H), |
| | | 2.12-1.95 (m, 1H) |
| I- | 780.5 | 11.63 (s, 1H), 11.14 (s, 1H), 8.13 (s, 1H), 7.71-7.67 (m, 1H), 7.42 (d, J = 9.2 Hz, |
| 167 | | 2H), 7.34-7.26 (m, 3H), 7.22 (d, J = 7.6 Hz, 1H), 7.14 (s, 1H), 7.13-7.08 (m, 1H), |
| | | 6.53-6.50 (m, 1H), 6.29 (br s, 1H), 5.44 (dd, J = 5.6, 12.8 Hz, 1H), 4.66-4.62 (m, |
| | | 2H), 4.43-4.36 (m, 2H), 3.80-3.78 (m, 6H), 3.64-3.57 (m, 2H), 3.17-3.09 (m, |
| | | 4H), 2.93-2.87 (m, 1H), 2.77-2.71 (m, 1H), 2.67 (br s, 2H), 2.63 (br s, 1H), 2.55- |
| | | 2.53 (m, 1H), 2.33 (br s, 2H), 2.29-2.23 (m, 1H), 2.10-2.03 (m, 1H) |
| I- | 732.5 | 11.53-11.47 (m, 1H), 10.75 (s, 1H), 8.18-8.10 (m, 2H), 7.71-7.68 (m, 1H), 7.26- |
| 168 | | 7.24 (m, 1H), 7.04-6.98 (m, 4H), 6.92 (br s, 1H), 6.39 (d, J = 8.0 Hz, 2H), 6.26 (br |
| | | s, 1H), 4.64 (br t, J = 6.8 Hz, 2H), 4.38 (br s, 1H), 4.34 (br s, 1H), 3.95-3.92 (m, 2H), |
| | | 3.71-3.68 (m, 1H), 3.62 (brt, J = 5.3 Hz, 1H), 3.57 (br t, J = 5.5 Hz, 2H), 3.48-3.44 |
| | | (m, 4H), 3.15 (br s, 1H), 3.13 (br d, J = 4.0 Hz, 1H), 3.10 (br s, 1H), 3.09 (br s, 1H), |
| | | 3.02 (br s, 1H), 2.99 (br s, 2H), 2.97-2.96 (m, 1H), 2.95 (br s, 1H), 2.68 (br d, J = 6.5 |
| | | Hz, 2H), 2.63-2.58 (m, 1H), 2.48-2.46 (m, 1H), 2.45-2.41 (m, 1H), 2.33 (br s, 1H), |
| | | 2.24 (br d, J = 7.0 Hz, 2H), 2.14-2.09 (m, 1H), 2.00 (br dd, J = 4.3, 8.8 Hz, 1H), 1.84 |
| | | (br d, J = 1.0 Hz, 2H) |
| I- | 751.6 | 10.75 (s, 1H), 8.11 (br d, J = 9.2 Hz, 1H), 7.69 (br d, J = 8.4 Hz, 1H), 7.47 (s, 1H), |
| 171 | | 7.30-7.21 (m, 1H), 7.00 (br d, J = 8.0 Hz, 2H), 6.39 (br d, J = 8.0 Hz, 2H), 6.09 (br |
| | | s, 1H), 4.65-4.60 (m, 2H), 4.33-4.26 (m, 2H), 3.92 (br t, J = 7.2 Hz, 2H), 3.69 (br |
| | | dd, J = 5.2, 10.8 Hz, 1H), 3.65-3.57 (m, 2H), 3.45 (br t, J = 6.0 Hz, 2H), 3.25 (br s, |
| | | 3H), 3.12-3.07 (m, 2H), 3.04 (br s, 3H), 2.97 (br d, J = 9.6 Hz, 2H), 2.95-2.89 (m, |
| | | 2H), 2.68-2.56 (m, 4H), 2.34-2.25 (m, 2H), 2.10 (br s, 2H), 1.84 (br s, 6H) |
| I- | 802.9 | 11.55-11.43 (m, 1H), 11.05 (s, 1H), 8.20 (s, 1H), 8.15-8.10 (m, 1H), 7.73-7.64 (m, |
| 176 | | 1H), 7.29-7.21 (m, 1H), 7.06-6.98 (m, 1H), 6.96-6.86 (m, 2H), 6.35-6.23 (m, |
| | | 2H), 6.11 (dd, J = 1.6, 8.4 Hz, 1H), 5.27 (br dd, J = 5.2, 12.8 Hz, 1H), 4.64 (br t, J = |
| | | 6.8 Hz, 2H), 4.43-4.30 (m, 2H), 3.95 (br t, J = 7.2 Hz, 2H), 3.65-3.55 (m, 3H), 3.45 |
| | | (br t, J = 6.0 Hz, 3H), 3.28 (s, 3H), 3.13 (td, J = 6.8, 18.0 Hz, 4H), 3.03-2.83 (m, |
| | | 6H), 2.58 (br s, 4H), 2.39-2.13 (m, 5H), 2.02-1.94 (m, 1H), 1.91-1.72 (m, 4H) |
| I- | 749.5 | 11.94 (s, 1H), 10.81 (s, 1H), 8.17 (s, 1H), 7.29-7.03 (m, 5H), 6.91-6.80 (m, 1H), |
| 178 | | 6.65 (s, 1H), 6.58 (d, J = 8.4 Hz, 1H), 6.48 (s, 1H), 6.09 (s, 1H), 4.44-4.21 (m, 2H), |
| | | 3.81 (dd, J = 4.0, 10.8 Hz, 2H), 3.71 (s, 3H), 3.66-3.34 (m, 4H), 3.20-2.80 (m, 6H), |
| | | 2.61 (t. J = 7.2 Hz, 4H), 2.53 (s, 3H), 2.46 (d, J = 4.5 Hz, 2H), 2.37 (s, 3H), 2.25 (s, |
| | | 1H), 2.17 (d, J = 10.2 Hz, 1H), 2.07 (d, J = 7.2 Hz, 4H), 1.88-1.66 (m, 2H) |
| I- | 855.5 | 12.22-12.12 (m, 1H), 10.85-10.77 (m, 1H), 8.37-8.26 (m, 1H), 7.76-7.67 (m, |
| 193 | | 1H), 7.57-7.50 (m, 2H), 7.47-7.36 (m, 1H), 7.25-7.20 (m, 2H), 7.14 (d, J = 8.0 Hz, |
| | | 2H), 7.09-7.04 (m, 2H), 7.03-6.94 (m, 2H), 6.24-6.15 (m, 1H), 4.54-4.40 (m, |
| | | 3H), 4.38-4.30 (m, 2H), 3.93-3.86 (m, 1H), 3.84 (s, 1H), 3.23 (br s, 6H), 3.10-2.97 |
| | | (m, 2H), 2.95-2.87 (m, 2H), 2.86-2.72 (m, 3H), 2.72-2.66 (m, 1H), 2.66-2.61 (m, |
| | | 5H), 2.58 (br d, J = 6.8 Hz, 3H), 2.20-2.13 (m, 1H), 2.06-2.00 (m, 1H), 1.91 (br d, |
| | | J = 3.2 Hz, 2H), 1.80-1.74 (m, 1H), 1.53-1.41 (m, 1H). |
| I- | 856.3 | 10.81 (s, 1H), 7.76-7.68 (m, 3H), 7.58 (d, J = 6.8 Hz, 1H), 7.41 (d, J = 17.6 Hz, 1H), |
| 194 | | 7.22 ( d, J = 8.0 Hz, 2H), 7.17-7.12 (m, 2H), 7.08 (d, J = 8.0 Hz, 2H), 6.24-6.16 (m, |
| | | 1H), 4.53-4.43 (m, 2H), 4.39-4.33 (m, 2H), 3.94-3.78 (m, 3H), 3.28-3.21 (m, |
| | | 5H), 3.12-3.02 (m, 2H), 2.98-2.86 (m, 4H), 2.57 (s, 11H), 2.23-2.14 (m, 1H), |
| | | 2.06-1.90 (m, 3H), 1.82-1.72 (m, 1H), 1.60-1.39 (m, 1H) |
| | | LC-MS (ESI+) m/z 856.3(M + H)+ |
| I- | 888.4 | 12.14 (br d, J = 5.2 Hz, 1H), 11.06 (s, 1H), 8.17-8.06 (m, 1H), 7.79-7.65 (m, 1H), |
| 201 | | 7.12 (br s, 1H), 6.91 (d, J = 8.4 Hz, 1H), 6.87-6.78 (m, 1H), 6.32 (d, J = 2.0 Hz, 1H), |
| | | 6.16-6.06 (m, 2H), 5.28 (dd, J = 5.2, 13.2 Hz, 1H), 4.63 (q, J = 6.4 Hz, 2H), 4.47 (br |
| | | d, J = 7.2 Hz, 2H), 4.38-4.26 (m, 2H), 3.95 (t, J = 7.2 Hz, 2H), 3.70-3.58 (m, 2H), |
| | | 3.48-3.38 (m, 4H), 3.29 (s, 3H), 3.10 (q, J = 7.2 Hz, 2H), 3.00-2.85 (m, 5H), 2.72- |
| | | 2.59 (m, 5H), 2.40-2.32 (m, 2H), 2.27 (br d, J = 3.6 Hz, 1H), 2.18 (br t, J = 10.4 |
| | | Hz, 2H), 2.01-1.92 (m, 1H), 1.84-1.72 (m, 3H) |
| I- | 777.3 | 12.03 (br s, 1H), 11.10 (s, 1H), 7.52 (br d, J = 8.0 Hz, 2H), 7.08 (br d, J = 8.0 Hz, 2H), |
| 218 | | 7.05-6.98 (m, 3H), 6.97-6.93 (m, 1H), 6.85 (br s, 1H), 5.44-5.33 (m, 1H), 4.53- |
| | | 4.43 (m, 1H), 3.93-3.83 (m, 1H), 3.67-3.59 (m, 3H), 3.25 (br s, 4H), 3.19-3.08 (m, |
| | | 6H), 2.98-2.83 (m, 2H), 2.76-2.72 (m, 1H), 2.71-2.63 (m, 8H), 2.55 (br s, 3H), |
| | | 2.04 (d, J = 10.8 Hz, 3H), 2.01-1.97 (m, 1H), 1.96-1.86 (m, 2H), 1.84-1.74 (m, |
| | | 1H), 1.66-1.40 (m, 1H) |
| I- | 859.4 | 11.99 (br d, J = 8 Hz, 1H), 11.06 (br d, J = 2.0 Hz, 1H), 7.80-7.67 (m, 1H), 7.48- |
| 231 | | 7.36 (m, 1H), 6.95-6.86 (m, 1H), 6.84-6.76 (m, 2H), 6.32 (d, J = 2.0 Hz, 1H), |
| | | 6.23-6.17 (m, 1H), 6.14-6.04 (m, 2H), 5.27 (dd, J = 5.2, 12.8 Hz, 1H), 4.42-4.18 |
| | | (m, 4H), 3.94 (t, J = 7.2 Hz, 2H), 3.64 (br s, 4H), 3.58 (br t, J = 5.6 Hz, 1H), 3.45 |
| | | (br t, J = 6.4 Hz, 2H), 3.29 (s, 3H), 3.01-2.92 (m, 5H), 2.62 (br d, J = 6.4 Hz, 3H), |
| | | 2.34-2.31 (m, 1H), 2.28-2.21 (m, 1H), 2.19-2.12 (m, 2H), 2.01-1.95 (m, 1H), |
| | | 1.81 (br d, J = 2.0 Hz, 3H), 1.66 (br d, J = 4.8 Hz, 2H), 1.56 (br s, 4H), 1.36 (s, 2H), |
| | | 1.25-1.19 (m, 3H) |
| I- | 789.4 | 12.12-11.85 (m, 1H), 10.75 (s, 1H), 7.77-7.68 (m, 1H), 7.48-7.35 (m, 1H), 7.01 |
| 251 | | (d, J = 8.4 Hz, 2H), 6.84-6.75 (m, 2H), 6.39 (d, J = 8.4 Hz, 2H), 6.24-6.16 (m, 1H), |
| | | 6.07 (s, 1H), 4.40-4.26 (m, 4H), 3.93 (t, J = 7.2 Hz, 2H), 3.72-3.55 (m, 8H), 3.46 |
| | | (t, J = 6.4 Hz, 2H), 3.01-2.88 (m, 6H), 2.64 (d, J = 6.8 Hz, 2H), 2.40-2.23 (m, 4H), |
| | | 2.20-2.13 (m, 2H), 2.00 (J = 4.8, 13.4 Hz, 1H), 1.85-1.74 (m, 4H), 1.66 (d, J = 4.4 |
| | | Hz, 2H), 1.56 (s, 4H) |
| I- | 870.4 | 12.17 (s, 1H), 10.75 (s, 1H), 7.71 (d, J = 11.6 Hz, 1H), 7.52 (d, J = 8.4 Hz, 2H), 7.41 |
| 280 | | (d, J = 16.4 Hz, 1H), 7.06 (d, J = 8.4 Hz, 2H), 7.03-6.94 (m, 4H), 6.61 (d, J = 8.4 Hz, |
| | | 2H), 6.20 (d, J = 10.4 Hz, 1H), 5.64 (d, J = 8.0 Hz, 1H), 4.52-4.41 (m, 3H), 4.41- |
| | | 4.20 (m, 4H), 3.92-3.85 (m, 1H), 3.22 (s, 4H), 3.11-3.01 (m, 2H), 2.92 (td, J = 7.6, |
| | | 14.4 Hz, 3H), 2.82-2.74 (m, 3H), 2.60 (s, 4H), 2.12 (dd, J = 4.4, 8.0 Hz, 1H), 2.03- |
| | | 1.97 (m, 1H), 1.94-1.83 (m, 3H), 1.80-1.73 (m, 1H), 1.58-1.39 (m, 2H), 1.24 ( s, |
| | | 3H) |
| I- | 684.1 | 11.98 (s, 1H), 10.54 (s, 1H), 7.72 (dd, J = 1.6, 7.6 Hz, 1H), 7.58 (dd, J = 1.6, 8.0 Hz, |
| 378 | | 1H), 7.52-7.46 (m, 1H), 7.42 (d, J = 7.6 Hz, 1H), 7.34-7.25 (m, 2H), 7.00 (dd, J = |
| | | 5.6, 10.8 Hz, 1H), 6.53-6.48 (m, 1H), 4.54-4.39 (m, 1H), 3.86 (d, J = 12.4 Hz, 1H), |
| | | 3.82-3.78 (m, 3H), 3.78-3.73 (m, 1H), 3.65 (td, J = 6.4, 12.4 Hz, 1H), 3.23-2.93 |
| | | (m, 9H), 2.81-2.74 (m, 2H), 2.04 (d, J = 8.0 Hz, 3H), 1.94-1.72 (m, 3H), 1.62-1.42 |
| | | (m, 1H) |
| I- | 881.3 | 11.11 (s, 1H), 7.97-7.99 (m, 1H), 7.73-7.76 (m, 2H), 7.70-7.57 (m, 2H), 7.57- |
| 409 | | 7.41 (m, 2H), 7.40 (s, 1H), 7.16-7.04 (m, 3H), 7.03-6.89 (m, 2H), 6.60 (d, J = 9.2 |
| | | Hz, 1H), 6.47-6.49 (m, 1H), 6.18 (s, 1H), 5.50-5.30 (m, 1H), 4.37 (s, 2H), 3.72 (s, |
| | | 3H), 3.69-3.60 (m, 2H), 3.28-3.21 (m, 6H), 2.98-2.86 (m, 3H), 2.78-2.64 (m, |
| | | 2H), 2.58 (s, 4H), 2.48-2.38 (m, 2H), 2.38-2.23 (m, 2H), 2.07-1.98 (m, 1H) |
| I- | 720.4 | 12.24-12.11 (m, 1H), 10.94-10.63 (m, 1H), 7.38-7.31 (m, 1H), 7.17-7.12 (m, |
| 514 | | 1H), 7.06 (br t, J = 8.4 Hz, 2H), 7.03-6.95 (m, 4H), 6.84 (br d, J = 1.2 Hz, 1H), 6.61 |
| | | (d, J = 8.4 Hz, 2H), 6.23-6.10 (m, 1H), 5.74-5.59 (m, 1H), 4.36 (br d, J = 7.2 Hz, |
| | | 2H), 4.31-4.23 (m, 1H), 3.70-3.57 (m, 2H), 3.23 (br s, 7H), 3.15-2.99 (m, 4H), |
| | | 2.66-2.55 (m, 8H), 2.43-2.26 (m, 3H), 2.13-2.02 (m, 4H), 1.93-1.77 (m, 1H) |
| I- | 790.3 | 11.76 (br s, 1H), 8.16-8.06 (m, 1H), 7.68 (br d, J = 9.6 Hz, 1H), 7.55 (s, 1H), 7.44- |
| 673 | | 7.39 (m, 3H), 7.11-6.81 (m, 5H), 6.18-6.10 (m, 2H), 5.43-5.31 (m, 1H), 4.66- |
| | | 4.59 (m, 2H), 4.39-4.31 (m, 2H), 3.70 (s, 3H), 3.67-3.62 (m, 1H), 3.61-3.58 (m, |
| | | 1H), 3.25 (br d, J = 11.6 Hz, 4H), 3.10 (br s, 2H), 2.94-2.85 (m, 3H), 2.82-2.75 (m, |
| | | 1H), 2.69-2.63 (m, 1H), 2.34 (br d, J = 2.4 Hz, 1H), 2.27 (br d, J = 2.8 Hz, 1H), 1.96 |
| | | (br s, 4H) |
| I- | 830.3 | 12.19-12.10 (m, 1H), 11.16-11.05 (m, 1H), 7.48-7.42 (m, 1H), 7.16-7.12 (m, |
| 805 | | 1H), 7.05-6.97 (m, 4H), 6.97-6.88 (m, 1H), 6.57-6.52 (m, 1H), 6.16-6.10 (m, |
| | | 1H), 5.39 (br dd, J = 5.2, 12.8 Hz, 1H), 4.35-4.30 (m, 2H), 4.01-3.91 (m, 2H), |
| | | 3.67-3.57 (m, 5H), 3.53-3.46 (m, 1H), 3.20-3.02 (m, 6H), 3.00-2.87 (m, 3H), 2.77- |
| | | 2.69 (m, 1H), 2.60 (br s, 1H), 2.38 (br s, 1H), 2.27 (br d, J = 1.6 Hz, 1H), 2.07 (d, J = |
| | | 8.8 Hz, 3H), 2.04-1.95 (m, 3H), 1.93-1.84 (m, 2H) |
| I- | 781.1 | 11.93 (s, 1H), 11.10 (s, 1H), 7.05-7.01 (m, 1H), 6.99 (br d, J = 5.0 Hz, 2H), 6.96- |
| 909 | | 6.90 (m, 1H), 6.80-6.73 (m, 1H), 6.09-6.02 (m, 1H), 5.42-5.34 (m, 1H), 4.31 (br |
| | | s, 2H), 3.60 (s, 3H), 3.58 (br s, 1H), 3.25-3.02 (m, 8H), 3.01-2.86 (m, 3H), 2.78- |
| | | 2.63 (m, 4H), 2.58 (br dd, J = 7.6, 15.6 Hz, 7H), 2.37 (br s, 2H), 2.23 (br s, 1H), |
| | | 2.11-1.98 (m, 9H), 1.59-1.51 (m, 4H) |
| I- | 873.1 | 12.21-12.16 (m, 1H), 11.12-11.07 (m, 1H), 7.63 (d, J = 8.4 Hz, 1H), 7.16 (br d, J = |
| 913 | | 8.4 Hz, 1H), 7.10-7.01 (m, 1H), 6.99 (d, J = 5.2 Hz, 2H), 6.97-6.93 (m, 1H), 6.57 |
| | | (d, J = 2.4 Hz, 1H), 6.16 (br s, 1H), 5.41-5.35 (m, 1H), 4.38-4.32 (m, 2H), 3.65- |
| | | 3.59 (m, 5H), 3.31-3.29 (m, 3H), 3.20-3.18 (m, 3H), 3.15-3.09 (m, 4H), 3.02 (br |
| | | s, 4H), 2.97-2.84 (m, 2H), 2.77-2.68 (m, 3H), 2.68-2.59 (m, 5H), 2.38 (br d, J = |
| | | 2.4 Hz, 1H), 2.32-2.22 (m, 1H), 2.08 (d, J = 6.8 Hz, 3H), 2.04-1.98 (m, 1H) |
| I- | 826.4 | 12.20 (s, 1H), 10.80 (s, 1H), 7.72 (dd, J = 2.0, 10.2 Hz, 1H), 7.50-7.36 (m, 3H), |
| 942 | | 7.23-7.16 (m, 1H), 7.08-6.99 (m, 1H), 6.95 (dd, J = 8.0, 13.2 Hz, 1H), 6.88 (br s, 1H), |
| | | 6.78 (dd, J = 8.4, 14.4 Hz, 1H), 6.25-6.11 (m, 2H), 5.54 (d, J = 8.4 Hz, 1H), 4.46- |
| | | 4.27 (m, 5H), 3.70-3.53 (m, 2H), 3.26-3.17 (m, 6H), 3.07 (s, 7H), 3.01-2.93 (m, |
| | | 3H), 2.74 (dt, J = 6.4, 11.6 Hz, 1H), 2.62-2.54 (m, 2H), 2.38-2.24 (m, 2H), 2.10- |
| | | 1.99 (m, 2H) |
| I- | 789.3 | 12.31-12.13 (m, 1H), 11.12 (s, 1H), 7.68 (d, J = 8.0 Hz, 1H), 7.32 (d, J = 8.0 Hz, |
| 943 | | 1H), 7.05-6.97 (m, 2H), 6.97-6.86 (m, 2H), 6.44 (d, J = 2.4 Hz, 1H), 6.16 (d, J = |
| | | 1.2 Hz, 1H), 5.43-5.31 (m, 1H), 4.43-4.35 (m, 2H), 3.70 (s, 3H), 3.68-3.62 (m, |
| | | 2H), 3.28 (d, J = 12.4 Hz, 2H), 3.21-3.00 (m, 6H), 3.00-2.96 (m, 1H), 2.95-2.93 |
| | | (m, 1H), 2.88 (d, J = 10.8 Hz, 3H), 2.77-2.68 (m, 1H), 2.67-2.60 (m, 1H), 2.36 (s, |
| | | 4H), 2.24 (s, 1H), 2.04 (s, 4H), 2.02-1.98 (m, 1H), 1.07-1.00 (m, 6H) |
| I- | 854.5 | 12.22-12.02 (m, 1H), 11.17 (s, 1H), 7.69-7.62 (m, 1H), 7.46-7.23 (m, 4H), 7.05- |
| 944 | | 6.97 (m, 2H), 6.81 (d, J = 6.0 Hz, 2H), 6.42-6.38 (m, 1H), 6.22-6.09 (m, 2H), |
| | | 5.41-5.34 (m, 1H), 4.42-4.23 (m, 3H), 4.14-4.06 (m, 1H), 3.71 (s, 3H), 3.65-3.44 (m, |
| | | 3H), 3.29-3.22 (m, 3H), 3.21-2.99 (m, 6H), 2.95-2.86 (m, 3H), 2.79-2.65 (m, |
| | | 3H), 2.32-2.20 (m, 2H), 2.20-2.16 (m, 3H), 2.00-1.94 (m, 4H), 1.04-0.98 (m, 3H) |

### Example 8. STAT6 Assay Results

### STAT6 HTRF Results

HTRF binding assays were performed using 0.15 nM biotinylated truncated STAT6 (123-632)-avi purified from E. coli, 1× Streptavidin-terbium (CisBio) prepared by mixing SA-Tb in PPI detection buffer (CisBio), 20 nM proprietary fluorescein-labeled probe, and test compounds in assay buffer consisting of 50 mM HEPES-Na pH 7.5, 100 mM NaCl, 1 mM EDTA, 2 mM DTT, 0.1% Tween-20 with a final volume of 20 uL. Compound stocks were dissolved at 10 mM in 100% DMSO and 11 point titration with 3 fold serial dilution was performed in white, opaque 384 well microplates. Reaction plates were incubated at room temperature for 30 minutes. Plates were centrifuged at low rpm for 5 mins, and the ratio of fluorescence intensities were measured at emission wavelengths for fluorescein acceptor (520 nm) and terbium donor (495 nm) on Envision Plate reader. % Inhibition was calculated from the 520/495 ratio generated by using proprietary positive control compound for 100% inhibition and DMSO only reactions for 0% inhibition. Data was processed and dose response curves were generated using GraphPad Prism to determine the concentration required for inhibiting 50% of the HTRF signal (IC₅₀).

The STAT6 HTRF results are shown in Table 14. The letter codes for IC₅₀ (μM) include: A (<0.1 μM); B (0.1-1 μM); C (>1-10 μM); D (>10-100 μM); E (>100 μM); and F (not tested).

TABLE 14
STAT6 HTRF Results

| | STAT6 |
|---|---|
| | HTRF: |
| | Average |
| I-# | IC₅₀ (μM) |
| I-1 | A |
| I-2 | A |
| I-3 | B |
| I-4 | A |
| I-5 | C |
| I-6 | B |
| I-7 | B |
| I-8 | B |
| I-9 | A |
| I-10 | A |
| 1-11 | A |
| I-12 | A |
| I-13 | A |
| I-14 | A |
| I-15 | D |
| I-16 | F |
| I-17 | B |
| I-18 | C |
| I-19 | D |
| I-20 | C |
| I-21 | C |
| I-22 | A |
| I-23 | A |
| I-24 | A |
| I-25 | A |
| I-26 | A |
| I-27 | A |
| I-28 | A |
| I-29 | A |
| I-30 | A |
| I-31 | A |
| I-32 | A |
| I-33 | A |
| I-34 | A |
| I-35 | A |
| I-36 | A |
| I-37 | F |
| I-38 | F |
| I-39 | F |
| I-40 | A |
| I-41 | A |
| I-42 | B |
| I-43 | B |
| I-44 | B |
| I-45 | F |
| I-46 | B |
| I-47 | F |
| I-48 | F |
| I-49 | A |
| I-50 | C |
| I-51 | C |
| I-52 | A |
| I-53 | B |
| I-54 | D |
| I-55 | C |
| I-56 | D |
| I-57 | C |
| I-58 | C |
| I-59 | B |
| I-60 | D |
| I-62 | A |
| I-63 | B |
| I-64 | A |
| I-65 | F |
| I-66 | F |
| I-67 | B |
| I-68 | D |
| I-69 | D |
| I-70 | D |
| I-71 | D |
| I-72 | D |
| I-73 | D |
| I-74 | D |
| I-75 | D |
| I-76 | D |
| I-77 | C |
| I-78 | D |
| I-79 | C |
| I-80 | B |
| I-81 | B |
| I-82 | B |
| I-83 | B |
| I-84 | B |
| I-85 | A |
| I-86 | D |
| I-87 | D |
| I-88 | D |
| I-89 | D |
| 1-90 | D |
| I-91 | D |
| I-92 | D |
| I-93 | D |
| I-94 | D |
| I-95 | D |
| I-96 | D |
| I-97 | A |
| I-99 | F |
| I-100 | A |
| I-101 | A |
| I-102 | A |
| I-103 | A |
| I-104 | B |
| I-106 | F |
| I-107 | F |
| I-108 | F |
| I-109 | F |
| I-110 | D |
| I-111 | A |
| I-112 | A |
| I-113 | D |
| I-114 | A |
| I-115 | A |
| I-116 | A |
| I-117 | A |
| I-118 | A |
| I-119 | A |
| I-120 | A |
| I-121 | A |
| I-122 | A |
| I-123 | A |
| I-124 | A |
| I-125 | A |
| I-126 | A |
| I-127 | A |
| I-128 | A |
| I-129 | A |
| I-130 | B |
| I-131 | A |
| I-132 | A |
| I-133 | A |
| I-134 | A |
| I-135 | B |
| I-136 | A |
| I-137 | B |
| I-138 | A |
| I-139 | A |
| I-140 | A |
| I-141 | A |
| I-142 | A |
| I-143 | A |
| I-144 | A |
| I-145 | C |
| I-146 | A |
| I-147 | A |
| I-148 | A |
| I-149 | A |
| I-150 | A |
| I-151 | A |
| I-152 | A |
| I-153 | A |
| I-154 | A |
| I-155 | A |
| I-156 | A |
| I-157 | A |
| I-158 | A |
| I-159 | B |
| I-160 | A |
| I-161 | A |
| I-162 | A |
| I-163 | A |
| I-164 | A |
| I-165 | A |
| I-166 | A |
| I-167 | A |
| I-168 | A |
| I-169 | A |
| I-170 | A |
| I-171 | A |
| I-172 | C |
| I-173 | A |
| I-174 | B |
| I-175 | C |
| I-176 | A |
| I-177 | A |
| I-178 | A |
| I-179 | C |
| I-180 | A |
| I-181 | A |
| I-182 | D |
| I-183 | B |
| I-184 | C |
| I-185 | C |
| I-186 | D |
| I-187 | D |
| I-188 | D |
| I-189 | A |
| I-190 | B |
| I-191 | C |
| I-192 | A |
| I-193 | C |
| I-194 | D |
| I-195 | A |
| I-196 | B |
| I-197 | C |
| I-198 | B |
| I-199 | D |
| I-200 | B |
| I-201 | B |
| I-202 | C |
| I-203 | D |
| I-204 | F |
| I-205 | D |
| I-206 | D |
| I-207 | C |
| I-208 | D |
| I-209 | C |
| I-210 | C |
| I-211 | D |
| I-212 | D |
| I-213 | C |
| I-214 | D |
| I-215 | C |
| I-216 | A |
| I-217 | A |
| I-218 | B |
| I-219 | C |
| I-220 | B |
| I-221 | A |
| I-222 | A |
| I-223 | D |
| I-224 | A |
| I-225 | B |
| I-226 | A |
| I-227 | B |
| I-228 | D |
| I-229 | B |
| I-230 | B |
| I-231 | B |
| I-232 | A |
| I-233 | B |
| I-234 | A |
| I-235 | B |
| I-236 | A |
| I-237 | C |
| I-238 | A |
| I-239 | C |
| I-240 | A |
| I-241 | D |
| I-242 | A |
| I-243 | A |
| I-244 | C |
| I-245 | D |
| I-246 | D |
| I-247 | A |
| I-248 | A |
| I-249 | A |
| I-250 | A |
| I-251 | B |
| I-252 | A |
| I-253 | A |
| I-254 | A |
| I-255 | A |
| I-256 | C |
| I-257 | C |
| I-258 | C |
| I-259 | A |
| I-260 | A |
| I-261 | A |
| I-262 | B |
| I-263 | B |
| I-264 | A |
| I-265 | B |
| I-266 | C |
| I-267 | A |
| I-268 | C |
| I-269 | D |
| I-270 | C |
| I-271 | B |
| I-272 | D |
| I-273 | C |
| I-274 | A |
| I-275 | A |
| I-276 | B |
| I-277 | A |
| I-278 | B |
| I-279 | D |
| I-280 | C |
| I-281 | B |
| I-282 | C |
| I-283 | A |
| I-284 | D |
| I-285 | D |
| I-286 | A |
| I-287 | C |
| I-288 | B |
| I-289 | D |
| I-290 | B |
| I-291 | D |
| I-292 | C |
| I-293 | D |
| I-294 | C |
| I-295 | D |
| I-296 | D |
| I-297 | B |
| I-298 | C |
| I-299 | C |
| I-300 | C |
| I-301 | C |
| I-302 | C |
| I-303 | D |
| I-304 | B |
| I-305 | C |
| I-306 | D |
| I-307 | C |
| I-308 | C |
| I-309 | A |
| I-310 | C |
| I-311 | C |
| I-312 | D |
| I-313 | B |
| I-314 | D |
| I-315 | B |
| I-316 | A |
| I-317 | B |
| I-318 | C |
| I-319 | B |
| I-320 | C |
| I-321 | C |
| I-322 | D |
| I-323 | D |
| I-324 | A |
| I-325 | D |
| I-326 | C |
| I-327 | D |
| I-328 | B |
| I-329 | C |
| I-330 | A |
| I-331 | C |
| I-332 | D |
| I-333 | D |
| I-334 | B |
| I-335 | C |
| I-336 | B |
| I-337 | B |
| I-338 | C |
| I-339 | A |
| I-340 | B |
| I-341 | A |
| I-342 | B |
| I-343 | C |
| I-344 | D |
| I-345 | C |
| I-346 | D |
| I-347 | B |
| I-348 | C |
| I-349 | B |
| I-350 | C |
| I-351 | A |
| I-352 | D |
| I-353 | C |
| I-354 | C |
| I-355 | A |
| I-356 | C |
| I-357 | B |
| I-358 | B |
| I-359 | B |
| I-360 | B |
| I-361 | D |
| I-362 | C |
| I-363 | C |
| I-364 | C |
| I-365 | C |
| I-366 | C |
| I-367 | C |
| I-368 | B |
| I-369 | A |
| I-370 | B |
| I-371 | A |
| I-372 | A |
| I-373 | A |
| I-374 | A |
| I-375 | B |
| I-376 | A |
| I-377 | B |
| I-378 | C |
| I-379 | C |
| I-380 | C |
| I-381 | A |
| I-382 | C |
| I-383 | A |
| I-384 | A |
| I-385 | B |
| I-386 | B |
| I-387 | D |
| I-388 | B |
| I-389 | B |
| I-390 | B |
| I-391 | B |
| I-392 | B |
| I-393 | C |
| I-394 | C |
| I-395 | C |
| I-396 | A |
| I-397 | D |
| I-398 | C |
| I-399 | C |
| I-400 | A |
| I-401 | B |
| I-402 | A |
| I-403 | B |
| I-404 | C |
| I-405 | B |
| I-406 | C |
| I-407 | B |
| I-408 | A |
| I-409 | B |
| I-410 | C |
| I-411 | B |
| I-412 | B |
| I-413 | B |
| I-414 | B |
| I-415 | B |
| I-416 | D |
| I-417 | C |
| I-418 | B |
| I-419 | B |
| I-420 | C |
| I-421 | C |
| I-422 | C |
| I-423 | A |
| I-424 | B |
| I-425 | C |
| I-426 | B |
| I-427 | B |
| I-428 | B |
| I-429 | A |
| I-430 | B |
| I-431 | B |
| I-432 | B |
| I-433 | A |
| I-434 | B |
| I-435 | B |
| I-436 | C |
| I-437 | D |
| I-438 | A |
| I-439 | C |
| I-440 | C |
| I-441 | A |
| I-442 | C |
| I-443 | A |
| I-444 | B |
| I-445 | B |
| I-446 | B |
| I-447 | C |
| I-448 | C |
| I-449 | B |
| I-450 | B |
| I-451 | B |
| I-452 | A |
| I-453 | B |
| I-454 | C |
| I-455 | C |
| I-456 | B |
| I-457 | A |
| I-458 | B |
| I-459 | B |
| I-460 | B |
| I-461 | C |
| I-462 | B |
| I-463 | A |
| I-464 | A |
| I-465 | B |
| I-466 | A |
| I-467 | A |
| I-468 | A |
| I-469 | A |
| I-470 | A |
| I-471 | B |
| I-472 | B |
| I-473 | B |
| I-474 | B |
| I-475 | A |
| I-476 | B |
| I-477 | C |
| I-478 | A |
| I-479 | C |
| I-480 | A |
| I-481 | B |
| I-482 | B |
| I-483 | B |
| I-484 | B |
| I-485 | A |
| I-486 | B |
| I-487 | C |
| I-488 | A |
| I-489 | C |
| I-490 | B |
| I-491 | B |
| I-492 | B |
| I-493 | B |
| I-494 | A |
| I-495 | A |
| I-496 | B |
| I-497 | A |
| I-498 | C |
| I-499 | A |
| I-500 | A |
| I-501 | A |
| I-502 | A |
| I-503 | A |
| I-504 | A |
| I-505 | C |
| I-506 | D |
| I-507 | A |
| I-508 | A |
| I-509 | B |
| I-510 | C |
| I-511 | B |
| I-512 | A |
| I-513 | A |
| I-514 | B |
| I-515 | C |
| I-516 | D |
| I-517 | D |
| I-518 | B |
| I-519 | C |
| I-520 | C |
| I-521 | B |
| I-522 | A |
| I-523 | B |
| I-524 | B |
| I-525 | C |
| I-526 | A |
| I-527 | B |
| I-528 | D |
| I-529 | C |
| I-530 | C |
| I-531 | C |
| I-532 | B |
| I-533 | B |
| I-534 | B |
| I-535 | C |
| I-536 | B |
| I-537 | B |
| I-538 | C |
| I-539 | B |
| I-540 | A |
| I-541 | A |
| I-542 | B |
| I-543 | A |
| I-544 | A |
| I-545 | A |
| I-546 | A |
| I-547 | B |
| I-548 | C |
| I-549 | D |
| I-550 | C |
| I-551 | A |
| I-552 | A |
| I-553 | B |
| I-554 | B |
| I-555 | D |
| I-556 | C |
| I-557 | D |
| I-558 | D |
| I-559 | C |
| I-560 | A |
| I-561 | B |
| I-562 | B |
| I-563 | A |
| I-564 | B |
| I-565 | B |
| I-566 | C |
| I-567 | A |
| I-568 | A |
| I-569 | D |
| I-570 | A |
| I-571 | A |
| I-572 | A |
| I-573 | A |
| I-574 | A |
| I-575 | A |
| I-576 | A |
| I-577 | A |
| I-578 | A |
| I-579 | B |
| I-580 | C |
| I-581 | C |
| I-582 | A |
| I-583 | C |
| I-584 | A |
| I-585 | A |
| I-586 | D |
| I-587 | A |
| I-588 | A |
| I-589 | A |
| I-590 | A |
| I-591 | A |
| I-592 | A |
| I-593 | A |
| I-594 | B |
| I-595 | A |
| I-596 | A |
| I-597 | A |
| I-598 | B |
| I-599 | A |
| I-600 | A |
| I-601 | A |
| I-602 | C |
| I-603 | B |
| I-604 | A |
| I-605 | B |
| I-606 | D |
| I-607 | C |
| I-608 | B |
| I-609 | C |
| I-610 | D |
| I-611 | D |
| I-612 | B |
| I-613 | A |
| I-614 | B |
| I-615 | C |
| I-616 | B |
| I-617 | C |
| I-618 | C |
| I-619 | A |
| I-620 | C |
| I-621 | A |
| I-622 | A |
| I-623 | A |
| I-624 | A |
| I-625 | A |
| I-626 | B |
| I-627 | A |
| I-628 | A |
| I-629 | A |
| I-630 | B |
| I-631 | C |
| I-632 | C |
| I-633 | C |
| I-634 | C |
| I-635 | A |
| I-636 | A |
| I-637 | B |
| I-638 | C |
| I-639 | B |
| I-640 | B |
| I-641 | D |
| I-642 | D |
| I-643 | C |
| I-644 | D |
| I-645 | C |
| I-646 | C |
| I-647 | C |
| I-648 | C |
| I-649 | C |
| I-650 | B |
| I-651 | A |
| I-652 | B |
| I-653 | A |
| I-654 | D |
| I-655 | D |
| I-656 | D |
| I-657 | D |
| I-658 | B |
| I-659 | A |
| I-660 | B |
| I-661 | A |
| I-662 | B |
| I-663 | D |
| I-664 | C |
| I-665 | C |
| I-666 | B |
| I-667 | C |
| I-668 | C |
| I-669 | A |
| I-670 | C |
| I-671 | C |
| I-672 | C |
| I-673 | C |
| I-674 | C |
| I-675 | A |
| I-676 | A |
| I-677 | A |
| I-678 | B |
| I-679 | B |
| I-680 | A |
| I-681 | A |
| I-682 | B |
| I-683 | B |
| I-684 | C |
| I-685 | C |
| I-686 | C |
| I-687 | B |
| I-688 | A |
| I-689 | A |
| I-690 | B |
| I-691 | C |
| I-692 | A |
| I-693 | A |
| I-694 | A |
| I-695 | A |
| I-696 | A |
| I-697 | A |
| I-698 | B |
| I-699 | A |
| I-700 | A |
| I-701 | A |
| I-702 | A |
| I-703 | F |
| I-704 | A |
| I-705 | F |
| I-706 | A |
| I-707 | C |
| I-708 | C |
| I-709 | A |
| I-710 | A |
| I-711 | B |
| I-712 | D |
| I-713 | B |
| I-714 | B |
| I-715 | A |
| I-716 | A |
| I-717 | B |
| I-718 | C |
| I-719 | A |
| I-720 | D |
| I-721 | C |
| I-722 | D |
| I-723 | C |
| I-724 | D |
| I-725 | A |
| I-726 | A |
| I-727 | A |
| I-728 | A |
| I-729 | A |
| I-730 | A |
| I-731 | A |
| I-732 | C |
| I-733 | A |
| I-734 | A |
| I-735 | A |
| I-736 | A |
| I-737 | B |
| I-738 | A |
| I-739 | B |
| I-740 | B |
| I-741 | A |
| I-742 | A |
| I-743 | A |
| I-744 | A |
| I-745 | A |
| I-746 | B |
| I-747 | B |
| I-748 | B |
| I-749 | B |
| I-750 | A |
| I-751 | B |
| I-752 | B |
| I-753 | C |
| I-754 | A |
| I-755 | B |
| I-756 | B |
| I-757 | A |
| I-758 | A |
| I-759 | A |
| I-760 | C |
| I-761 | A |
| I-762 | A |
| I-763 | A |
| I-764 | B |
| I-765 | C |
| I-766 | B |
| I-767 | C |
| I-768 | A |
| I-769 | A |
| I-770 | A |
| I-771 | A |
| I-772 | B |
| I-773 | A |
| I-774 | B |
| I-775 | A |
| I-776 | A |
| I-777 | C |
| I-778 | A |
| I-779 | A |
| I-780 | A |
| I-781 | A |
| I-782 | A |
| I-783 | A |
| I-784 | A |
| I-785 | A |
| I-786 | B |
| I-787 | D |
| I-788 | A |
| I-789 | A |
| I-790 | C |
| I-791 | C |
| I-792 | A |
| I-793 | A |
| I-794 | F |
| I-795 | A |
| I-796 | A |
| I-797 | A |
| I-798 | A |
| I-799 | F |
| I-800 | A |
| I-801 | A |
| I-802 | A |
| I-803 | C |
| I-804 | C |
| I-805 | F |
| I-806 | C |
| I-807 | D |
| I-808 | A |
| I-809 | A |
| I-810 | D |
| I-811 | D |
| I-812 | C |
| I-813 | B |
| I-814 | B |
| I-815 | D |
| I-816 | B |
| I-817 | B |
| I-818 | C |
| I-819 | B |
| I-820 | C |
| I-821 | C |
| I-822 | C |
| I-823 | C |
| I-824 | A |
| I-825 | A |
| I-826 | C |
| I-827 | B |
| I-828 | B |
| I-829 | F |
| I-830 | A |
| I-831 | A |
| I-832 | A |
| I-833 | C |
| I-834 | C |
| I-835 | A |
| I-836 | B |
| I-837 | C |
| I-838 | A |
| I-839 | B |
| I-840 | D |
| I-841 | C |
| I-842 | A |
| I-843 | C |
| I-844 | B |
| I-845 | B |
| I-846 | B |
| I-847 | A |
| I-848 | B |
| I-849 | C |
| I-850 | D |
| I-851 | C |
| I-852 | D |
| I-853 | C |
| I-854 | D |
| I-855 | B |
| I-856 | A |
| I-857 | A |
| I-858 | C |
| I-859 | B |
| I-860 | A |
| I-861 | A |
| I-862 | C |
| I-863 | D |
| I-864 | C |
| I-865 | B |
| I-866 | B |
| I-867 | B |
| I-868 | A |
| I-869 | C |
| I-870 | D |
| I-871 | C |
| I-872 | C |
| I-873 | C |
| I-874 | B |
| I-875 | B |
| I-876 | C |
| I-877 | A |
| I-878 | C |
| I-879 | D |
| I-880 | A |
| I-881 | A |
| I-882 | A |
| I-883 | B |
| I-884 | B |
| I-885 | C |
| I-886 | A |
| I-887 | A |
| I-888 | C |
| I-889 | C |
| I-890 | B |
| I-891 | D |
| I-892 | C |
| I-893 | A |
| I-894 | C |
| I-895 | B |
| I-896 | C |
| I-897 | A |
| I-898 | C |
| I-899 | C |
| I-900 | D |
| I-901 | C |
| I-902 | F |
| I-903 | A |
| I-904 | A |
| I-905 | A |
| I-906 | A |
| I-907 | A |
| I-908 | B |
| I-909 | A |
| I-910 | C |
| I-911 | A |
| I-912 | B |
| I-913 | C |
| I-914 | B |
| I-915 | D |
| I-916 | D |
| I-917 | D |
| I-918 | C |
| I-919 | F |
| I-920 | F |
| I-921 | C |
| I-922 | C |
| I-923 | C |
| I-924 | D |
| I-925 | A |
| I-926 | A |
| I-927 | D |
| I-928 | B |
| I-929 | B |
| I-930 | D |
| I-931 | F |
| I-932 | C |
| I-933 | F |
| I-934 | A |
| I-935 | B |
| I-936 | D |
| I-937 | D |
| I-938 | A |
| I-939 | A |
| I-940 | A |
| I-941 | A |
| I-942 | A |
| I-943 | B |
| I-944 | C |
| I-945 | B |
| I-946 | A |
| I-947 | B |
| I-948 | A |
| I-949 | F |
| I-950 | A |
| I-951 | F |
| I-952 | F |
| I-953 | F |
| I-954 | F |
| I-955 | F |
| I-956 | F |
| I-957 | F |
| I-958 | F |
| I-959 | F |
| I-960 | F |
| I-961 | A |
| I-962 | C |
| I-963 | C |
| I-964 | D |
| I-965 | C |
| I-966 | B |
| I-967 | C |
| I-968 | B |
| I-969 | B |
| I-970 | B |
| I-971 | B |
| I-972 | B |
| I-973 | B |
| I-974 | D |
| I-975 | A |
| I-976 | A |
| I-977 | A |
| I-978 | C |
| I-979 | A |
| I-980 | A |
| I-981 | A |
| I-982 | C |
| I-983 | D |
| I-984 | A |
| I-985 | C |
| I-986 | A |
| I-987 | B |
| I-988 | B |
| I-989 | D |
| 1-990 | A |
| I-991 | A |
| I-992 | D |
| I-993 | A |
| I-994 | A |
| I-995 | A |
| I-996 | A |
| I-997 | A |
| I-998 | A |
| I-999 | A |
| I-1000 | A |
| I-1001 | A |
| I-1002 | A |
| I-1003 | A |
| I-1004 | A |
| I-1005 | A |
| I-1006 | A |
| I-1007 | F |
| I-1008 | B |
| I-1009 | F |
| I-1010 | A |
| I-1011 | A |
| I-1012 | A |
| I-1013 | A |
| I-1014 | A |
| I-1015 | A |
| I-1016 | F |
| I-1017 | A |
| I-1018 | B |
| I-1019 | A |
| I-1020 | A |
| I-1021 | B |
| I-1022 | A |
| I-1023 | A |
| I-1024 | A |
| I-1025 | B |
| I-1026 | A |
| I-1027 | A |
| I-1028 | A |
| I-1029 | A |
| I-1030 | A |
| I-1031 | A |
| I-1032 | A |
| I-1033 | A |
| I-1034 | A |
| I-1035 | A |
| I-1036 | A |
| I-1037 | A |
| I-1038 | A |
| I-1039 | A |
| I-1040 | A |
| I-1041 | A |
| I-1042 | A |
| I-1043 | A |
| I-1044 | A |
| I-1045 | A |
| I-1046 | B |
| I-1047 | A |
| I-1048 | A |
| I-1049 | A |
| I-1050 | A |
| I-1051 | A |
| I-1052 | A |
| I-1053 | A |
| I-1054 | A |
| I-1055 | A |
| I-1056 | A |
| I-1057 | A |
| I-1058 | A |
| I-1059 | A |
| I-1060 | A |
| I-1061 | A |
| I-1062 | A |
| I-1063 | A |
| I-1064 | A |
| I-1065 | A |
| I-1066 | A |
| I-1067 | A |
| I-1068 | A |
| I-1069 | A |
| I-1070 | A |
| I-1071 | A |
| I-1072 | F |
| I-1073 | A |
| I-1074 | A |
| I-1075 | F |
| I-1076 | A |
| I-1077 | A |
| I-1078 | A |
| I-1079 | A |
| I-1080 | B |
| I-1081 | C |
| I-1082 | B |
| I-1083 | C |
| I-1084 | A |
| I-1085 | A |
| I-1086 | A |
| I-1087 | A |
| I-1088 | A |
| I-1089 | A |
| I-1090 | A |
| I-1091 | B |
| I-1092 | A |
| I-1093 | A |
| I-1094 | C |
| I-1095 | A |
| I-1096 | C |
| I-1097 | A |
| I-1098 | A |
| I-1099 | A |
| I-1100 | A |
| I-1101 | A |
| I-1102 | A |
| I-1103 | A |
| I-1104 | A |
| I-1105 | C |
| I-1106 | B |
| I-1107 | A |
| I-1108 | A |
| I-1109 | A |
| I-1110 | A |
| I-1111 | A |
| I-1112 | B |
| I-1113 | A |
| I-1114 | B |
| I-1115 | B |
| I-1116 | A |
| I-1117 | A |
| I-1118 | A |
| I-1119 | A |
| I-1120 | A |
| I-1121 | A |
| I-1122 | A |
| I-1123 | A |
| I-1124 | B |
| I-1125 | A |
| I-1126 | A |
| I-1127 | A |
| I-1128 | A |
| I-1129 | B |
| I-1130 | B |
| I-1131 | A |
| I-1132 | B |
| I-1133 | A |
| I-1134 | A |
| I-1135 | A |
| I-1136 | A |
| I-1137 | A |
| I-1138 | B |
| I-1139 | A |
| I-1140 | B |
| I-1141 | B |
| I-1142 | B |
| I-1143 | A |
| I-1144 | A |
| I-1145 | A |
| I-1146 | A |
| I-1147 | A |
| I-1148 | A |
| I-1149 | A |
| I-1150 | A |
| I-1151 | A |
| I-1152 | B |
| I-1153 | A |
| I-1154 | A |
| I-1155 | A |
| I-1156 | A |
| I-1157 | B |
| I-1158 | B |
| I-1159 | B |
| I-1160 | B |
| I-1161 | B |
| I-1162 | A |
| I-1163 | B |
| I-1164 | B |
| I-1165 | B |
| I-1166 | A |
| I-1167 | A |
| I-1168 | B |
| I-1169 | A |
| I-1170 | B |
| I-1171 | A |
| I-1172 | C |
| I-1173 | A |
| I-1174 | A |
| I-1175 | B |
| I-1176 | A |
| I-1177 | A |
| I-1178 | D |
| I-1179 | A |
| I-1180 | B |
| I-1181 | C |
| I-1182 | A |
| I-1183 | A |
| I-1184 | A |
| I-1185 | B |
| I-1186 | A |
| I-1187 | A |
| I-1188 | A |
| I-1189 | A |
| I-1190 | A |
| I-1191 | A |
| I-1192 | A |
| I-1193 | A |
| I-1194 | A |
| I-1195 | A |
| I-1196 | A |
| I-1197 | A |
| I-1198 | A |
| I-1199 | A |
| I-1200 | A |
| I-1201 | A |
| I-1202 | A |
| I-1203 | A |
| I-1204 | A |
| I-1205 | A |
| I-1206 | A |
| I-1207 | A |
| I-1208 | A |
| I-1209 | A |
| I-1210 | A |
| I-1211 | A |
| I-1212 | B |
| I-1213 | A |
| I-1214 | A |
| I-1215 | A |
| I-1216 | A |
| I-1217 | A |
| I-1218 | A |
| I-1219 | A |
| I-1220 | A |
| I-1221 | B |
| I-1222 | B |
| I-1223 | A |
| I-1224 | A |
| I-1225 | A |
| I-1226 | A |
| I-1227 | A |
| I-1228 | A |
| I-1229 | A |
| I-1230 | A |
| I-1231 | A |
| I-1232 | A |
| I-1233 | A |
| I-1234 | A |
| I-1235 | A |
| I-1236 | A |
| I-1237 | A |
| I-1238 | A |
| I-1239 | A |
| I-1240 | A |
| I-1241 | A |
| I-1242 | A |
| I-1243 | A |
| I-1244 | A |
| I-1245 | A |
| I-1246 | A |
| I-1247 | B |
| I-1248 | A |
| I-1249 | A |
| I-1250 | A |
| I-1251 | A |
| I-1252 | A |
| I-1253 | A |
| I-1254 | A |
| I-1255 | A |
| I-1256 | A |
| I-1257 | A |
| I-1258 | A |
| I-1259 | A |
| I-1260 | A |
| I-1261 | C |
| I-1262 | A |
| I-1263 | C |
| I-1264 | A |
| I-1265 | A |
| I-1266 | F |
| I-1267 | A |
| I-1268 | A |
| I-1269 | B |
| I-1273 | F |
| I-1274 | F |
| I-1275 | F |
| I-1276 | A |
| I-1277 | A |
| I-1278 | A |
| I-1279 | A |
| I-1280 | F |
| I-1281 | F |
| I-1283 | A |
| I-1284 | A |
| I-1285 | A |
| I-1286 | F |
| I-1287 | A |
| I-1288 | D |
| I-1289 | A |
| I-1290 | B |
| I-1291 | F |
| I-1294 | F |
| I-1295 | A |
| I-1296 | F |
| I-1297 | A |
| I-1298 | F |
| I-1299 | A |
| I-1300 | C |
| I-1301 | A |
| I-1303 | B |
| I-1304 | F |
| I-1305 | F |
| I-1306 | B |
| I-1307 | F |
| I-1308 | B |
| I-1310 | F |
| I-1311 | A |
| I-1312 | F |
| I-1313 | A |
| I-1314 | A |
| I-1315 | B |
| I-1316 | A |
| I-1317 | A |
| I-1318 | A |
| I-1319 | A |
| I-1320 | F |
| I-1321 | F |
| I-1322 | B |
| I-1327 | B |
| I-1328 | F |
| I-1329 | F |
| I-1330 | F |
| I-1331 | F |
| I-1335 | B |
| I-1337 | C |
| I-1339 | C |
| I-1341 | C |
| I-1343 | F |
| I-1344 | F |
| I-1345 | F |
| I-1346 | F |
| I-1347 | F |
| I-1348 | A |
| I-1349 | F |
| I-1350 | F |
| I-1351 | F |
| I-1352 | F |
| I-1353 | A |
| I-1354 | F |
| I-1355 | F |
| I-1356 | A |
| I-1357 | A |
| I-1358 | A |
| I-1359 | F |
| I-1360 | F |
| I-1361 | A |
| I-1362 | F |
| I-1363 | F |
| I-1364 | A |
| I-1365 | F |
| I-1366 | F |
| I-1367 | F |
| I-1368 | F |
| I-1369 | F |
| I-1370 | A |
| I-1371 | F |
| I-1372 | F |
| I-1373 | A |
| I-1374 | A |
| I-1375 | A |
| I-1376 | A |
| I-1377 | A |
| I-1378 | F |
| I-1379 | A |
| I-1380 | A |
| I-1381 | C |
| I-1382 | F |
| I-1383 | F |
| I-1384 | F |
| I-1385 | F |
| I-1386 | F |
| I-1387 | F |
| I-1388 | A |
| I-1389 | A |
| I-1390 | A |
| I-1391 | F |
| I-1392 | A |
| I-1393 | F |
| I-1394 | F |
| I-1395 | A |
| I-1396 | A |
| I-1397 | A |
| I-1398 | F |
| I-1399 | A |
| I-1400 | F |
| I-1402 | F |
| I-1403 | D |
| I-1404 | C |
| I-1405 | C |
| I-1406 | F |
| I-1407 | A |
| I-1408 | A |
| I-1409 | A |
| I-1410 | A |
| I-1411 | C |
| I-1412 | C |
| I-1413 | F |
| I-1414 | F |
| I-1415 | F |
| I-1416 | B |
| I-1417 | A |
| I-1418 | B |
| I-1419 | A |
| I-1420 | A |
| I-1421 | A |
| I-1422 | A |
| I-1423 | A |
| I-1424 | A |
| I-1425 | A |
| I-1426 | A |
| I-1427 | A |
| I-1428 | D |
| I-1429 | C |
| I-1430 | B |
| I-1431 | A |
| I-1432 | C |
| I-1433 | B |
| I-1434 | A |
| I-1435 | A |
| I-1436 | A |
| I-1437 | A |
| I-1438 | A |
| I-1439 | A |
| I-1440 | A |
| I-1441 | A |
| I-1442 | A |
| I-1443 | A |
| I-1444 | A |
| I-1445 | A |
| I-1446 | A |
| I-1447 | A |
| I-1448 | F |
| I-1449 | F |
| I-1450 | F |
| I-1451 | A |
| I-1452 | A |
| I-1453 | C |
| I-1454 | A |
| I-1455 | A |
| I-1456 | B |
| I-1457 | F |
| I-1461 | B |
| I-1462 | F |
| I-1463 | A |
| I-1464 | F |
| I-1465 | A |
| I-1466 | F |
| I-1467 | F |
| I-1468 | F |
| I-1469 | A |
| I-1470 | B |
| I-1471 | F |
| I-1475 | A |
| I-1476 | F |
| I-1477 | C |
| I-1478 | C |
| I-1479 | C |
| I-1480 | D |
| I-1481 | C |
| I-1482 | B |
| I-1483 | B |
| I-1484 | A |
| I-1485 | A |
| I-1486 | B |
| I-1487 | B |
| I-1488 | A |
| I-1489 | A |
| I-1490 | A |
| I-1491 | A |
| I-1492 | F |
| I-1493 | F |
| I-1494 | F |
| I-1495 | F |
| I-1496 | B |
| I-1497 | A |
| I-1498 | A |
| I-1499 | D |
| I-1500 | A |
| I-1501 | A |
| I-1502 | A |
| I-1503 | A |
| I-1504 | A |
| I-1505 | A |
| I-1506 | F |
| I-1507 | A |
| I-1508 | A |
| I-1509 | A |
| I-1510 | A |
| I-1511 | A |
| I-1512 | A |
| I-1513 | B |
| I-1514 | A |
| I-1515 | A |
| I-1516 | A |
| I-1517 | A |
| I-1518 | A |
| I-1519 | A |
| I-1520 | A |
| I-1521 | A |
| I-1522 | A |
| I-1523 | A |
| I-1524 | A |
| I-1525 | C |
| I-1526 | A |
| I-1527 | A |
| I-1528 | A |
| I-1529 | E |
| I-1530 | C |
| I-1531 | D |
| I-1532 | D |
| I-1533 | A |
| I-1534 | C |
| I-1535 | C |
| I-1537 | F |
| I-1538 | A |
| I-1539 | A |
| I-1540 | F |
| I-1541 | F |
| I-1542 | C |
| I-1543 | A |
| I-1544 | B |
| I-1545 | B |
| I-1546 | A |
| I-1547 | C |
| I-1548 | C |

### Example 9 STAT6 Ubiquitination

This example describes ubiquitination of STAT6 by compounds disclosed herein. Human STAT6 contains the following amino acid sequence:

| Amino Acid Sequence of the Human Canonical Isoform of STAT6 (Uniprot | |
|---|---|
| ID P42226-1) | |
| SEQ ID NO: 1 | |
| MSLWGLVSKMPPEKVQRLYVDFPQHLRHLLGDWLESQPWEFLVGSDAFCCNLASALLSDT | 60 |
| VQHLQASVGEQGEGSTILQHISTLESIYQRDPLKLVATFRQILQGEKKAVMEQFRHLPMP | 120 |
| FHWKQEELKFKTGLRRLQHRVGEIHLLREALQKGAEAGQVSLHSLIETPANGTGPSEALA | 180 |
| MLLQETTGELEAAKALVLKRIQIWKRQQQLAGNGAPFEESLAPLQERCESLVDIYSQLQQ | 240 |
| EVGAAGGELEPKTRASLTGRLDEVLRTLVTSCFLVEKQPPQVLKTQTKFQAGVRFLLGLR | 300 |
| FLGAPAKPPLVRADMVTEKQARELSVPQGPGAGAESTGEIINNTVPLENSIPGNCCSALF | 360 |
| KNLLLKKIKRCERKGTESVTEEKCAVLFSASFTLGPGKLPIQLQALSLPLVVIVHGNQDN | 420 |
| NAKATILWDNAFSEMDRVPFVVAERVPWEKMCETLNLKFMAEVGTNRGLLPEHELFLAQK | 480 |
| IFNDNSLSMEAFQHRSVSWSQFNKEILLGRGFTFWQWFDGVLDLTKRCLRSYWSDRLIIG | 540 |
| FISKQYVTSLLLNEPDGTFLLRFSDSEIGGITIAHVIRGQDGSPQIENIQPFSAKDLSIR | 600 |
| SLGDRIRDLAQLKNLYPKKPKDEAFRSHYKPEQMGKDGRGYVPATIKMTVERDQPLPTPE | 660 |
| LQMPTMVPSYDLGMAPDSSMSMQLGPDMVPQVYPPHSHSIPPYQGLSPEESVNVLSAFQE | 720 |
| PHLQMPPSLGQMSLPFDQPHPQGLLPCQPQEHAVSSPDPLLCSDVTMVEDSCLSQPVTAF | 780 |
| PQGTWIGEDIFPPLLPPTEQDLTKLLLEGQGESGGGSLGAQPLLQPSHYGQSGISMSHMD | 840 |
| LRANPSW | 847 |

PBMC Sample Preparation Protocol. Frozen PBMC (AllCells LP, CR, MNC, 100M) were thawed and washed with prewarned complete media (RPMI 1640+10% HIFBS+Pen/Strep prepared with RPMI 1640 (Gibco. #11-875-093, #15140-122 and R&D Systems, #S11550H)). The cells were resuspended in complete media at 1.25 million cells per mL in flasks and incubated overnight in a tissue culture incubator.

The next day, 0.72 μL or 2.4 μL of a 10 mM stock solution of degrader compound (10 mM of I-1, I-167, I-205, I-383, or I-452 in DMSO) was added to a final concentration of 30 nM or 100 nM. At the end of treatment (15 minutes), the cell culture was poured into 50 mL tubes (10 tubes for each 480 mL total volume). The 50 mL tubes were spun down at 4000×g for 2 minutes. The media was decanted, and cells washed with 30 mL PBS. All the cells were combined, and then spun again at 4000×g for 2 minutes. Wash solution was decanted. All tubes were washed once more with 25 mL PBS and combined into one tube. 200 μL the cells (0.8%) was set aside for quality control analysis. The rest of the cells were spun down at 4000×g for 2 minutes. The second wash solution was decanted, and cells spun down again at 4000×g for 1 minute. A 1 mL pipette was used to remove all PBS from the pellet of cells. The pellet of cells was snap frozen for 20 seconds in liquid nitrogen, then stored at −78° C.

Extract protein, reduce, and alkylate: 1800 μL of Urca buffer (8 M Urca, 100 mM Triethylammonium bicarbonate (TEAB). 1 mM ethylenediaminetetraacetic acid (EDTA), 1% Halt™ Protease and Phosphotase Inhibitor (Thermo Fisher Scientific), and 1% Pierce™ Universal Nuclease (Thermo Fisher Scientific) was added to each cell sample. Samples were probe sonicated for 15 seconds (3 times) using 40% amplitude. Protein concentrations of the resultant cell lysates were determined by using Pierce™ Rapid Gold BCA Protein Assay Kit (Product No. A53226).

The lysate mixture containing 20 mg total proteins was reduced at RT for 30 min by adding dithiothreitol (DTT) to the final concentration of 10 mM DTT. 2-iodo acetamid (IAA) was then added to a final concentration of 10 mM to alkylate the proteins. The lysate mixture was placed in the dark for 30 mins, then a further amount of DTT to again reach 10 mM final concentration was added to the lysate mixture to neutralize excess IAA. The lysate mixture was incubated at room temperature for 5 min, then 100 mM TEAB buffer at pH 8.0 was added to dilute the lysate mixture to a urea concentration of less than IM. Trypsin/Lys-C Protease was then added to the reduced and alkylated lysate mixture to provide a protein to enzyme ratio of 1:50. The mixture was digested at 37° C. overnight in an incubator with 500 rpm shaking. The mixture was then acidified to a pH of lower than 3 using trifluoro acetic acid (TFA) and gently mixed to quench the digestion process.

Peptide Desalting: 200 mg Sep-Pak C18 cartridges (Waters) were activated by adding twice 750 μL of methanol (MeOH). Cartridges were equilibrated by twice letting 700 μL 0.1% formic acid (FA) pass through as above. The acidified sample mixture was added to the cartridges and collected after passing through the cartridge. Cartridges were washed three times with 700 μL of 0.1% formic acid. Peptides were eluted using 700 L volumes of 50% acetonitrile (ACN)⁺0.1% TFA and dried in a speed-vac concentrator.

DiGly Peptide Enrichment: Peptide samples containing 20 mg/sample were resuspended in 1.8 mL of 1×HS IAP Bind Buffer #1 from PTMScan® HS Ubiquitin/SUMO Remnant Motif (K-GG) Kit (#59322S). To that end, pellets were broken up mechanically by pipetting repeatedly, followed by shaking at room temperature for 5 min or placement in a sonicator bath for 2 min to ensure complete solubilization. The pH of peptide solutions was adjusted to 7.0 with 1M pH 7.0 TRIS base solution. Next, peptide solutions were clarified by centrifugation for 5 min at 10,000×g at 4° C., and cooled on ice. Insoluble residuals were discarded. 100 μL of diGly antibody bead slurry (20 L bed volume from PTMScan Kit referenced above) were used for each sample and washed five times with cold PBS. These antibodies recognize the Lys-ϵ-Gly-Gly (diGly) remnant that is generated following trypsin digestion of ubiquitylated proteins. Clarified peptide solution was added to beads and incubated on an end-over-end rotator overnight at 4° C. Beads were washed four times with 1 mL HS IAP Wash Buffer and twice with 1 mL H₂O. DiGly modified peptides were eluted from beads with 100 L of IAP Elution Buffer (0.15% TFA) for 20 min and again briefly with 100 μL of IAP Elution buffer. Pooled eluates were desalted by AttractSPE Disks Tips—C18 (AffiniSep) and eluted in 80 μL 80% ACN/0.2% TFA. Eluates were dried down.

Mass Spectrometry & Data Analysis: Samples were reconstituted in 5 μL 5% ACN/0.2% TFA, and 4 μL injected using the autosampler of an EasyLC 1200 nanoLC system onto a 40 cm analytical column with 75 m inner diameter packed in house with 1.9 m Dr Maisch Reprosil Pur AQ C18 material. Mobile phase solvents for reverse phase chromatography were H₂O+0.1% FA (Buffer A) and H₂O+90% acetonitrile+0.1% FA (Buffer B). A 125 min gradient was used with a constant flow of 300 nL/min starting at 3% Buffer B and ending at 32% Buffer B. Analytes eluting from the column were analyzed with an electrospray ionization voltage set to 1900V on a Thermo Exploris™ 480 mass spectrometer in positive mode with data dependent acquisition using the default Thermo template for label-free peptide MS2. The peptides in these samples were separated using reverse phase liquid chromatography and ionized using electrospray ionization. (These peptide ions enter a mass spectrometer where their mass-to-charge ratio and relative signal intensities are measured. These peptide ions are subsequently fragmented using High energy Collision-induced Dissociation (HCD) and the mass-to-charge ratios and signal intensities of these fragments is measured. This data is recorded is then recorded in MS raw files). Proteome Discoverer 3.1 with Sequest was used for proteomic searches with default peptide search parameters unless stated otherwise. Searches included fixed carbamidomethylation modification on cysteines, variable oxidation on methionine, variable acetylation of the protein N-terminus, and variable Lys-ϵ-Gly-Gly (diGly, or GlyGly, or GG) modification of lysines. Spectra were searched against a complete human Swiss-Prot sequence database (version downloaded on Jan. 15^{th} 2019) containing canonical protein sequences and isoforms.

TABLE 15A

| | Endogenous | | I-1 | | I-167 | |
|---|---|---|---|---|---|---|
| | Intensity | % of Total | Intensity | % of Total | Intensity | % of Total |
| | (E + 06) | STAT6 Ub | (E + 06) | STAT6 Ub | (E + 06) | STAT6 Ub |
| K94 | | | 2.31 ± 0.23 | 1.5 ± 0.2 | 4.37 ± 4.04 | 1.1 ± 0.4 |
| K107 | 0.15 ± 0.26 | 1.42 ± 2.46 | | | 0.42 ± 0.85 | 0.0 ± 0.1 |
| K108 | 0.21 ± 0.36 | 1.01 ± 1.74 | 1.97 ± 1.93 | 1.2 ± 1.2 | 0.51 ± 0.72 | 0.1 ± 0.1 |
| K124 | 0.47 ± 0.81 | 4.37 ± 7.57 | 0.22 ± 0.39 | 0.1 ± 0.2 | 0.80 ± 1.61 | 0.1 ± 0.2 |
| K277 | | | 0.64 ± 0.57 | 0.4 ± 0.4 | 0.56 ± 0.47 | 0.2 ± 0.2 |
| K284 | | | 0.41 ± 0.13 | 0.3 ± 0.1 | 1.09 ± 1.12 | 0.3 ± 0.1 |
| K288 | | | 1.23 ± 0.69 | 0.8 ± 0.5 | 2.64 ± 2.4 | 0.7 ± 0.2 |
| K307 | 2.06 ± 1.83 | 15.49 ± 16.76 | 5.78 ± 0.91 | 3.7 ± 0.9 | 14.2 ± 11.8 | 3.7 ± 0.3 |
| K319 | 4.24 ± 4.34 | 26.62 ± 23.16 | 8.12 ± 5.02 | 5.1 ± 3.1 | 41.9 ± 37.9 | 10.2 ± 2.6 |
| K374 | 0.5 ± 0.87 | 2.43 ± 4.21 | 2.73 ± 1.21 | 1.7 ± 0.7 | 3.80 ± 7.58 | 2.9 ± 5.8 |
| K504 | | | 54.1 ± 13.1 | 33.7 ± 6.4 | 162 ± 126 | 43.7 ± 4.4 |
| K595 | 0.25 ± 0.43 | 1.2 ± 2.08 | 10.6 ± 1.1 | 6.6 ± 0.2 | 33.2 ± 34.2 | 7.8 ± 1.2 |
| K614 | 1.14 ± 0.18 | 9.37 ± 4.18 | 1.44 ± 0.26 | 0.9 ± 0.2 | 2.14 ± 2.58 | 0.4 ± 0.1 |
| K630 | 4.9 ± 0.81 | 38.09 ± 12.37 | 52.0 ± 3.0 | 32.7 ± 2.2 | 87.2 ± 92.2 | 20.2 ± 3.2 |
| K636 | | | 5.33 ± 2.06 | 3.3 ± 1.2 | 8.02 ± 5.86 | 2.2 ± 0.5 |
| K647 | | | 12.4 ± 1.7 | 7.9 ± 1.7 | 28.0 ± 29.1 | 6.3 ± 2.1 |

TABLE 15B

| | I-205 | | I-383 | | I-452 | |
|---|---|---|---|---|---|---|
| | Intensity | | Intensity | | Intensity | |
| | (E + 06) | % of Total | (E + 06) | % of Total | (E + 06) | % of Total |
| K94 | 12.4 ± 0.5 | 3.2 ± 0.3 | 7.40 ± 2.12 | 1.5 ± 0.3 | 6.33 ± 0.74 | 2.7 ± 0.5 |
| K107 | 2.85 ± 0.41 | 0.7 ± 0.1 | 2.67 ± 0.71 | 0.5 ± 0.2 | 2.54 ± 1.19 | 1.1 ± 0.4 |
| K108 | 41.9 ± 3.8 | 10.7 ± 0.4 | 13.6 ± 4.6 | 2.8 ± 0.8 | 2.88 ± 1.25 | 1.2 ± 0.6 |
| K124 | 4.15 ± 0.22 | 1.1 ± 0.1 | 2.94 ± 0.62 | 0.6 ± 0.2 | 4.91 ± 1.17 | 2.0 ± 0.2 |
| K277 | 0.80 ± 1.38 | 0.2 ± 0.3 | 1.27 ± 2.20 | 0.2 ± 0.4 | 0 | 0.0 ± 0.0 |
| K284 | 1.82 ± 0.42 | 0.5 ± 0.1 | 2.15 ± 0.70 | 0.5 ± 0.2 | 2.01 ± 0.46 | 0.8 ± 0.1 |
| K288 | 6.82 ± 1.10 | 1.8 ± 0.3 | 9.40 ± 1.51 | 1.9 ± 0.4 | 8.46 ± 2.96 | 3.5 ± 0.7 |
| K307 | 9.79 ± 0.75 | 2.5 ± 0.2 | 10.1 ± 1.1 | 2.1 ± 0.3 | 7.84 ± 0.84 | 3.4 ± 0.8 |
| K319 | 17.5 ± 1.3 | 4.5 ± 0.5 | 27.4 ± 10.2 | 5.7 ± 2.5 | 23.7 ± 7.7 | 9.8 ± 1.6 |
| K504 | 101 ± 5 | 26.2 ± 2.5 | 206 ± 90 | 41.1 ± 12.6 | 89.9 ± 10.1 | 38.0 ± 2.6 |
| K595 | 14.4 ± 2.1 | 3.7 ± 0.3 | 23.1 ± 1.7 | 4.8 ± 0.4 | 12.9 ± 2.6 | 5.5 ± 1.0 |
| K614 | 5.70 ± 1.15 | 1.5 ± 0.3 | 4.94 ± 1.87 | 1.1 ± 0.5 | 5.72 ± 2.49 | 2.4 ± 0.8 |
| K618 | 3.86 ± 1.70 | 1.0 ± 0.4 | 1.60 ± 0.55 | 0.3 ± 0.1 | 3.86 ± 0.49 | 1.6 ± 0.1 |
| K630 | 129 ± 8 | 33.0 ± 0.1 | 138 ± 28 | 28.7 ± 7.6 | 37.8 ± 6.7 | 15.9 ± 0.5 |
| K636 | 3.95 ± 2.1 | 1.0 ± 0.5 | 5.53 ± 4.68 | 1.2 ± 1.1 | | |
| K647 | 33.1 ± 7.8 | 8.5 ± 1.5 | 34.2 ± 6.0 | 7.1 ± 1.7 | 29.3 ± 10.2 | 12.1 ± 3.2 |

Results: DiGly enriched peptides from cells treated with five exemplary compounds (I-1, 1-167, I-205, 1-383, I-452) provided the STAT6 ubiquitination profiles shown in Tables 15A, 15B, 15C. These profiles were distinct from profiles reported in the literature in untreated cancer cell lines (Steger. M., Demichev. V., Backman, M. et al. Time-resolved in vivo ubiquitinome profiling by DIA-MS reveals USP7 targets on a proteome-wide scale. Nat Commun 12, 5399 (2021) and Akimov, V, Barrio-Hernandez, I., Hansen. S.V.F. et al. UbiSite approach for comprehensive mapping of lysine and N-terminal ubiquitination sites. Nat Struct Mol Biol 25, 631-640), which are representative of the endogenous ubiquitination states of STAT6.

TABLE 15C

| | | | HepG2 | | Jurkat | |
|---|---|---|---|---|---|---|
| | HCT116 | | Intensity | | Intensity | |
| | Intensity (E + 06) | % of Total | (E + 06) | % of Total | (E + 06) | % of Total |
| K9 | 0.016 ± 0.046 | 11.8 ± 33.5 | | | | |
| K94 | 0.011 ± 0.005 | 81.5 ± 36.0 | | | | |
| K107 | | | | | 6.42 ± 8.91 | 14.9 ± 22.5 |
| K108 | 0.0007 ± 0.0018 | 6.7 ± 18.8 | | | | |
| K124 | | | 0.75 ± 1.30 | 33.3 ± 57.7 | 4.60 ± 7.97 | 6.0 ± 10.3 |
| K252 | | | 3.34 ± 5.79 | 15.9 ± 27.6 | 5.94 ± 7.58 | 9.6 ± 10.4 |
| K284 | | | 2.12 ± 3.67 | 10.1 ± 17.5 | 17.6 ± 5.5 | 28.1 ± 2.0 |
| K450 | | | | | 6.40 ± 5.55 | 12.0 ± 11.7 |
| K621 | | | 9.0 ± 15.4 | 33.8 ± 57.4 | 18.6 ± 16.6 | 25.5 ± 23.2 |
| K630 | | | 1.43 ± 2.48 | 6.8 ± 11.8 | 2.86 ± 4.95 | 3.7 ± 6.4 |
| K636 | | | | | 0.23 ± 0.39 | 0.3 ± 0.5 |

### Example 10 STAT6 MSD Degradation Results

Degradation of STAT6 in cells was quantitatively measured using Meso Scale Discovery (MSD) technology. A549 cells were seeded in 96-well plates with a density of 1.5×10e4 to 5×10e5 cells per well in 100 μl fresh media. Compounds were then added to the assay plates with a final top concentration of up to 10 uM in a 1:3 dilution series with total of 9 doses. The assay plates were then incubated for 4 to 24 hours at 37° C. under 5% CO₂. The assay plates were then centrifuged for 5 minutes and the cell pellets were treated with 100 μl/well RIPA lysis buffer (Boston Bioproducts, BP-115D) with protease and phosphatase inhibitors (Roche, 05892791001 and 04906837001). To prepare the MSD assay plates (MSD, L15XA), the plates were coated with the capture antibody (Abnova, H00006778) in PBS, at 40 μl/well. The plates were then incubated overnight at 4° C., washed 1 time with 150 μl/well TBST buffer (Cell Signaling Technology, #9997S) and blocked with 150 μl/well blocking buffer (MSD, R93BA-4). Cell lysates were then added to MSD assay plates and the plates were incubated at room temperature for 1 hour. The plates were then washed 3 times with 150 μl/well TBST buffer and 25 μl/well primary detection antibody (Cell Signaling Technology, #5397S). The assay plates were then incubated at room temperature for 1 hour, washed 3 times with 150 μl/well TBST buffer, and 25 μl/well secondary detection antibody, SULFO-TAG anti-rabbit antibody (MSD, R32AB-1) were added. The assay plates were then incubated at room temperature for 1 hour, washed 3 times with 150 μl/well TBST buffer, and 150 μl/well MSD reading buffer (MSD, R92TC-2) were added. The plates were then analyzed by an MSD reader. The data was analyzed by GraphPad Prism and the dose dependent STAT6 degradation was fit using the four-parameter inhibitor vs. response nonlinear regression.

The STAT6 MSD degradation results in A549 cells are shown in Table 16. The letter codes for DC₅₀ (nM) include: A (<1 nM); B (1-10 nM); C (>10-100 nM); D (>100-1000 nM); E (>1000 nM); and F (not tested). The letter codes for Dmax (oo) include: A (>90%); B (>70-90%); C (>50-70%); D (<500%); and F (not tested).

TABLE 16
STAT6 MSD Degradation Results

| | STAT6 MSD | |
|---|---|---|
| | degradation | STAT6 MSD |
| | DC50 (nM) | degradation |
| I-# | 24 h | Dmax % 24 h |
| I-1 | A | A |
| I-2 | A | A |
| I-3 | E | D |
| I-4 | E | C |
| I-5 | E | D |
| I-6 | D | B |
| I-7 | D | B |
| I-8 | D | B |
| I-9 | E | D |
| I-10 | E | D |
| I-11 | E | D |
| I-12 | D | B |
| I-13 | E | D |
| I-14 | E | D |
| I-15 | E | D |
| I-16 | F | E |
| I-17 | E | D |
| I-18 | E | D |
| I-19 | E | D |
| I-20 | E | D |
| I-21 | B | C |
| I-22 | D | C |
| I-23 | C | B |
| I-24 | D | B |
| I-25 | E | C |
| I-26 | E | C |
| I-27 | D | B |
| I-28 | C | B |
| I-29 | E | D |
| I-30 | D | B |
| I-31 | D | C |
| I-32 | D | C |
| I-33 | C | B |
| I-34 | E | D |
| I-35 | E | D |
| I-36 | E | D |
| I-37 | F | E |
| I-38 | F | E |
| I-39 | F | E |
| I-40 | C | B |
| I-41 | E | D |
| I-42 | E | D |
| I-43 | E | D |
| I-44 | E | D |
| I-45 | E | D |
| I-46 | E | D |
| I-47 | D | B |
| I-48 | D | C |
| I-49 | E | D |
| I-50 | E | D |
| I-51 | E | D |
| I-52 | C | C |
| I-53 | F | E |
| I-54 | E | D |
| I-55 | E | D |
| I-56 | E | D |
| I-57 | E | D |
| I-58 | E | D |
| I-59 | E | D |
| I-60 | E | D |
| I-62 | E | D |
| I-63 | C | D |
| I-64 | E | D |
| I-65 | E | D |
| I-66 | F | E |
| I-67 | F | E |
| I-68 | E | D |
| I-69 | E | D |
| I-70 | E | D |
| I-71 | E | D |
| I-72 | E | D |
| I-73 | E | D |
| I-74 | E | D |
| I-75 | F | E |
| I-76 | F | E |
| I-77 | E | D |
| I-78 | E | D |
| I-79 | C | C |
| I-80 | E | D |
| I-81 | E | D |
| I-82 | E | D |
| I-83 | E | D |
| I-84 | E | D |
| I-85 | E | D |
| I-86 | E | D |
| I-87 | E | D |
| I-88 | E | D |
| I-89 | E | D |
| I-90 | E | D |
| I-91 | E | D |
| I-92 | E | D |
| I-93 | E | D |
| I-94 | E | D |
| I-95 | E | D |
| I-96 | E | D |
| I-97 | B | A |
| I-99 | D | B |
| I-100 | D | B |
| I-101 | D | C |
| I-102 | D | B |
| I-103 | E | B |
| I-104 | B | C |
| I-106 | F | E |
| I-107 | F | E |
| I-108 | F | E |
| I-109 | F | E |
| I-110 | E | D |
| I-111 | D | C |
| I-112 | D | B |
| I-113 | C | D |
| I-114 | C | A |
| I-115 | D | B |
| I-116 | D | B |
| I-117 | D | D |
| I-118 | C | A |
| I-119 | D | B |
| I-120 | C | B |
| I-121 | C | C |
| I-122 | B | C |
| I-123 | C | B |
| I-124 | C | B |
| I-125 | E | D |
| I-126 | E | D |
| I-127 | E | D |
| I-128 | E | D |
| I-129 | C | B |
| I-130 | B | C |
| I-131 | C | D |
| I-132 | C | B |
| I-133 | D | C |
| I-134 | E | D |
| I-135 | B | A |
| I-136 | E | D |
| I-137 | C | C |
| I-138 | D | B |
| I-139 | C | D |
| I-140 | C | B |
| I-141 | E | D |
| I-142 | D | D |
| I-143 | B | C |
| I-144 | B | B |
| I-145 | E | D |
| I-146 | B | A |
| I-147 | E | D |
| I-148 | A | A |
| I-149 | D | D |
| I-150 | D | B |
| I-151 | E | D |
| I-152 | B | A |
| I-153 | E | D |
| I-154 | D | C |
| I-155 | E | D |
| I-156 | A | B |
| I-157 | B | A |
| I-158 | A | A |
| I-159 | E | D |
| I-160 | A | A |
| I-161 | A | A |
| I-162 | A | A |
| I-163 | A | A |
| I-164 | A | A |
| I-165 | A | A |
| I-166 | A | A |
| I-167 | A | A |
| I-168 | C | B |
| I-169 | B | A |
| I-170 | A | A |
| I-171 | C | C |
| I-172 | E | D |
| I-173 | A | A |
| I-174 | B | B |
| I-175 | E | D |
| I-176 | B | A |
| I-177 | A | A |
| I-178 | C | C |
| I-179 | B | D |
| I-180 | A | A |
| I-181 | C | A |
| I-182 | E | D |
| I-183 | B | B |
| I-184 | E | D |
| I-185 | B | D |
| I-186 | E | D |
| I-187 | E | D |
| I-188 | E | D |
| I-189 | A | A |
| I-190 | A | B |
| I-191 | B | D |
| I-192 | A | A |
| I-193 | C | B |
| I-194 | D | D |
| I-195 | A | A |
| I-196 | A | A |
| I-197 | E | D |
| I-198 | A | A |
| I-199 | E | D |
| I-200 | E | D |
| I-201 | C | B |
| I-202 | E | D |
| I-203 | E | D |
| I-204 | F | E |
| I-205 | E | D |
| I-206 | E | D |
| I-207 | E | D |
| I-208 | E | D |
| I-209 | E | D |
| I-210 | E | D |
| I-211 | E | D |
| I-212 | E | D |
| I-213 | E | D |
| I-214 | E | D |
| I-215 | E | D |
| I-216 | A | A |
| I-217 | A | A |
| I-218 | B | B |
| I-219 | E | D |
| I-220 | A | B |
| I-221 | A | A |
| I-222 | B | B |
| I-223 | E | D |
| I-224 | B | C |
| I-225 | A | C |
| I-226 | A | B |
| I-227 | E | D |
| I-228 | E | D |
| I-229 | C | C |
| I-230 | B | C |
| I-231 | B | B |
| I-232 | A | A |
| I-233 | C | C |
| I-234 | A | C |
| I-235 | B | C |
| I-236 | A | A |
| I-237 | B | B |
| I-238 | A | A |
| I-239 | E | D |
| I-240 | A | A |
| I-241 | E | D |
| I-242 | B | B |
| I-243 | A | A |
| I-244 | B | A |
| I-245 | E | D |
| I-246 | E | D |
| I-247 | A | A |
| I-248 | A | A |
| I-249 | E | D |
| I-250 | B | A |
| I-251 | C | B |
| I-252 | A | A |
| I-253 | B | A |
| I-254 | B | A |
| I-255 | B | B |
| I-256 | E | D |
| I-257 | E | D |
| I-258 | B | C |
| I-259 | A | A |
| I-260 | A | A |
| I-261 | B | B |
| I-262 | B | C |
| I-263 | B | B |
| I-264 | A | A |
| I-265 | A | A |
| I-266 | E | D |
| I-267 | A | A |
| I-268 | E | D |
| I-269 | E | D |
| I-270 | E | D |
| I-271 | B | B |
| I-272 | E | D |
| I-273 | E | D |
| I-274 | E | D |
| I-275 | E | D |
| I-276 | E | D |
| I-277 | C | D |
| I-278 | A | B |
| I-279 | E | D |
| I-280 | D | D |
| I-281 | C | B |
| I-282 | D | D |
| I-283 | A | B |
| I-284 | E | D |
| I-285 | E | D |
| I-286 | A | A |
| I-287 | C | B |
| I-288 | E | D |
| I-289 | E | D |
| I-290 | B | A |
| I-291 | E | D |
| I-292 | E | D |
| I-293 | E | D |
| I-294 | D | C |
| I-295 | E | D |
| I-296 | E | D |
| I-297 | A | A |
| I-298 | E | D |
| I-299 | E | D |
| I-300 | A | B |
| I-301 | B | A |
| I-302 | E | D |
| I-303 | E | D |
| I-304 | E | D |
| I-305 | A | B |
| I-306 | E | D |
| I-307 | B | C |
| I-308 | E | D |
| I-309 | E | D |
| I-310 | E | D |
| I-311 | B | A |
| I-312 | E | D |
| I-313 | C | B |
| I-314 | E | D |
| I-315 | A | A |
| I-316 | E | D |
| I-317 | C | B |
| I-318 | E | D |
| I-319 | E | D |
| I-320 | E | D |
| I-321 | E | D |
| I-322 | E | D |
| I-323 | E | D |
| I-324 | A | A |
| I-325 | E | D |
| I-326 | E | D |
| I-327 | E | D |
| I-328 | A | B |
| I-329 | E | D |
| I-330 | B | A |
| I-331 | B | B |
| I-332 | E | D |
| I-333 | E | D |
| I-334 | A | B |
| I-335 | A | B |
| I-336 | A | A |
| I-337 | A | A |
| I-338 | A | B |
| I-339 | C | B |
| I-340 | E | D |
| I-341 | A | A |
| I-342 | A | A |
| I-343 | E | D |
| I-344 | E | D |
| I-345 | E | D |
| I-346 | E | D |
| I-347 | A | B |
| I-348 | E | D |
| I-349 | C | C |
| I-350 | A | A |
| I-351 | B | B |
| I-352 | C | D |
| I-353 | E | D |
| I-354 | B | C |
| I-355 | B | A |
| I-356 | A | A |
| I-357 | A | A |
| I-358 | A | A |
| I-359 | A | A |
| I-360 | B | B |
| I-361 | E | D |
| I-362 | E | D |
| I-363 | B | B |
| I-364 | B | B |
| I-365 | A | B |
| I-366 | A | B |
| I-367 | B | B |
| I-368 | A | A |
| I-369 | A | A |
| I-370 | A | A |
| I-371 | A | A |
| I-372 | A | A |
| I-373 | A | A |
| I-374 | B | B |
| I-375 | B | C |
| I-376 | A | A |
| I-377 | A | A |
| I-378 | C | D |
| I-379 | C | B |
| I-380 | E | D |
| I-381 | A | A |
| I-382 | E | D |
| I-383 | A | A |
| I-384 | B | B |
| I-385 | C | C |
| I-386 | B | A |
| I-387 | C | C |
| I-388 | B | B |
| I-389 | B | A |
| I-390 | C | B |
| I-391 | A | A |
| I-392 | B | B |
| I-393 | E | D |
| I-394 | E | D |
| I-395 | E | D |
| I-396 | A | A |
| I-397 | E | D |
| I-398 | E | D |
| I-399 | E | D |
| I-400 | B | B |
| I-401 | C | B |
| I-402 | C | C |
| I-403 | E | D |
| I-404 | B | C |
| I-405 | B | B |
| I-406 | E | D |
| I-407 | A | B |
| I-408 | B | B |
| I-409 | B | D |
| I-410 | E | D |
| I-411 | B | B |
| I-412 | C | B |
| I-413 | B | B |
| I-414 | A | B |
| I-415 | A | B |
| I-416 | E | D |
| I-417 | E | D |
| I-418 | C | C |
| I-419 | B | B |
| I-420 | B | B |
| I-421 | E | D |
| I-422 | E | D |
| I-423 | A | B |
| I-424 | B | B |
| I-425 | C | D |
| I-426 | A | A |
| I-427 | A | B |
| I-428 | A | A |
| I-429 | A | A |
| I-430 | A | B |
| I-431 | B | B |
| I-432 | A | A |
| I-433 | C | B |
| I-434 | B | B |
| I-435 | A | B |
| I-436 | E | D |
| I-437 | E | D |
| I-438 | A | A |
| I-439 | C | C |
| I-440 | E | D |
| I-441 | B | B |
| I-442 | E | D |
| I-443 | B | A |
| I-444 | B | A |
| I-445 | C | A |
| I-446 | A | A |
| I-447 | B | A |
| I-448 | E | D |
| I-449 | B | B |
| I-450 | A | A |
| I-451 | C | B |
| I-452 | A | A |
| I-453 | B | A |
| I-454 | B | A |
| I-455 | A | A |
| I-456 | A | A |
| I-457 | B | B |
| I-458 | B | B |
| I-459 | B | B |
| I-460 | C | D |
| I-461 | B | C |
| I-462 | B | B |
| I-463 | A | B |
| I-464 | B | C |
| I-465 | E | D |
| I-466 | A | C |
| I-467 | E | D |
| I-468 | B | A |
| I-469 | B | A |
| I-470 | B | A |
| I-471 | C | B |
| I-472 | C | C |
| I-473 | B | B |
| I-474 | B | B |
| I-475 | B | B |
| I-476 | B | B |
| I-477 | C | B |
| I-478 | B | B |
| I-479 | B | B |
| I-480 | B | B |
| I-481 | C | D |
| I-482 | B | D |
| I-483 | C | B |
| I-484 | A | A |
| I-485 | B | D |
| I-486 | E | D |
| I-487 | E | D |
| I-488 | B | A |
| I-489 | B | B |
| I-490 | C | D |
| I-491 | A | B |
| I-492 | B | B |
| I-493 | C | B |
| I-494 | E | D |
| I-495 | C | C |
| I-496 | C | C |
| I-497 | E | D |
| I-498 | A | B |
| I-499 | A | A |
| I-500 | A | A |
| I-501 | D | C |
| I-502 | A | B |
| I-503 | A | B |
| I-504 | A | B |
| I-505 | B | B |
| I-506 | B | B |
| I-507 | A | A |
| I-508 | E | D |
| I-509 | B | B |
| I-510 | B | B |
| I-511 | E | D |
| I-512 | B | A |
| I-513 | C | D |
| I-514 | C | C |
| I-515 | B | D |
| I-516 | E | D |
| I-517 | E | D |
| I-518 | B | B |
| I-519 | A | A |
| I-520 | B | B |
| I-521 | B | B |
| I-522 | C | B |
| I-523 | C | B |
| I-524 | C | A |
| I-525 | E | D |
| I-526 | E | D |
| I-527 | C | C |
| I-528 | E | D |
| I-529 | C | C |
| I-530 | B | B |
| I-531 | A | A |
| I-532 | A | A |
| I-533 | B | A |
| I-534 | A | A |
| I-535 | A | A |
| I-536 | B | A |
| I-537 | C | C |
| I-538 | E | D |
| I-539 | C | D |
| I-540 | C | D |
| I-541 | B | A |
| I-542 | B | B |
| I-543 | B | B |
| I-544 | F | E |
| I-545 | A | B |
| I-546 | A | B |
| I-547 | E | D |
| I-548 | C | B |
| I-549 | E | D |
| I-550 | B | A |
| I-551 | C | B |
| I-552 | E | D |
| I-553 | C | B |
| I-554 | E | D |
| I-555 | C | B |
| I-556 | E | D |
| I-557 | E | D |
| I-558 | E | D |
| I-559 | E | D |
| I-560 | C | C |
| I-561 | B | B |
| I-562 | C | D |
| I-563 | E | D |
| I-564 | A | B |
| I-565 | C | D |
| I-566 | A | A |
| I-567 | E | D |
| I-568 | E | D |
| I-569 | C | C |
| I-570 | B | B |
| I-571 | A | A |
| I-572 | E | D |
| I-573 | C | D |
| I-574 | B | B |
| I-575 | E | D |
| I-576 | B | B |
| I-577 | B | B |
| I-578 | E | D |
| I-579 | E | D |
| I-580 | C | B |
| I-581 | C | A |
| I-582 | B | B |
| I-583 | E | D |
| I-584 | D | C |
| I-585 | B | B |
| I-586 | B | B |
| I-587 | C | C |
| I-588 | B | B |
| I-589 | B | B |
| I-590 | B | B |
| I-591 | E | D |
| I-592 | B | D |
| I-593 | E | D |
| I-594 | C | C |
| I-595 | B | C |
| I-596 | E | D |
| I-597 | E | D |
| I-598 | C | B |
| I-599 | E | D |
| I-600 | E | D |
| I-601 | B | B |
| I-602 | E | D |
| I-603 | C | C |
| I-604 | A | A |
| I-605 | E | D |
| I-606 | C | D |
| I-607 | E | D |
| I-608 | E | D |
| I-609 | D | C |
| I-610 | E | D |
| I-611 | E | D |
| I-612 | B | B |
| I-613 | A | A |
| I-614 | A | A |
| I-615 | A | A |
| I-616 | C | B |
| I-617 | A | A |
| I-618 | B | D |
| I-619 | B | B |
| I-620 | E | D |
| I-621 | B | C |
| I-622 | B | B |
| I-623 | E | D |
| I-624 | E | D |
| I-625 | E | D |
| I-626 | B | B |
| I-627 | E | D |
| I-628 | A | A |
| I-629 | B | D |
| I-630 | A | A |
| I-631 | A | A |
| I-632 | B | A |
| I-633 | C | C |
| I-634 | C | B |
| I-635 | A | A |
| I-636 | A | C |
| I-637 | A | A |
| I-638 | C | B |
| I-639 | A | A |
| I-640 | B | A |
| I-641 | C | D |
| I-642 | E | D |
| I-643 | B | A |
| I-644 | A | B |
| I-645 | A | A |
| I-646 | A | B |
| I-647 | B | A |
| I-648 | E | D |
| I-649 | A | A |
| I-650 | A | A |
| I-651 | E | D |
| I-652 | E | D |
| I-653 | E | D |
| I-654 | E | D |
| I-655 | E | D |
| I-656 | E | D |
| I-657 | E | D |
| I-658 | B | B |
| I-659 | A | A |
| I-660 | C | B |
| I-661 | B | B |
| I-662 | A | A |
| I-663 | C | C |
| I-664 | E | D |
| I-665 | E | D |
| I-666 | A | A |
| I-667 | B | A |
| I-668 | A | A |
| I-669 | B | B |
| I-670 | B | A |
| I-671 | B | A |
| I-672 | B | A |
| I-673 | B | B |
| I-674 | A | A |
| I-675 | B | B |
| I-676 | B | B |
| I-677 | A | A |
| I-678 | A | A |
| I-679 | B | A |
| I-680 | A | A |
| I-681 | B | A |
| I-682 | B | B |
| I-683 | B | A |
| I-684 | A | A |
| I-685 | A | A |
| I-686 | A | A |
| I-687 | A | A |
| I-688 | A | A |
| I-689 | B | A |
| I-690 | A | A |
| I-691 | C | B |
| I-692 | A | B |
| I-693 | C | B |
| I-694 | B | A |
| I-695 | B | A |
| I-696 | A | A |
| I-697 | A | A |
| I-698 | B | A |
| I-699 | A | B |
| I-700 | A | A |
| I-701 | A | A |
| I-702 | B | B |
| I-703 | C | C |
| I-704 | B | B |
| I-705 | C | B |
| I-706 | C | B |
| I-707 | B | B |
| I-708 | B | B |
| I-709 | B | C |
| I-710 | C | D |
| I-711 | B | B |
| I-712 | C | B |
| I-713 | C | B |
| I-714 | C | B |
| I-715 | C | B |
| I-716 | C | C |
| I-717 | B | D |
| I-718 | C | B |
| I-719 | B | B |
| I-720 | C | B |
| I-721 | C | C |
| I-722 | C | B |
| I-723 | C | C |
| I-724 | E | D |
| I-725 | A | A |
| I-726 | A | A |
| I-727 | A | A |
| I-728 | A | A |
| I-729 | A | A |
| I-730 | A | A |
| I-731 | B | B |
| I-732 | B | A |
| I-733 | A | A |
| I-734 | A | A |
| I-735 | B | A |
| I-736 | A | B |
| I-737 | B | B |
| I-738 | B | A |
| I-739 | A | A |
| I-740 | A | A |
| I-741 | A | A |
| I-742 | A | A |
| I-743 | A | A |
| I-744 | A | A |
| I-745 | A | A |
| I-746 | A | A |
| I-747 | A | A |
| I-748 | A | A |
| I-749 | B | A |
| I-750 | A | A |
| I-751 | A | A |
| I-752 | A | A |
| I-753 | B | A |
| I-754 | A | A |
| I-755 | B | A |
| I-756 | B | A |
| I-757 | A | A |
| I-758 | A | A |
| I-759 | A | A |
| I-760 | C | B |
| I-761 | A | B |
| I-762 | A | A |
| I-763 | B | A |
| I-764 | B | A |
| I-765 | B | A |
| I-766 | B | B |
| I-767 | C | B |
| I-768 | A | A |
| I-769 | A | A |
| I-770 | A | A |
| I-771 | B | D |
| I-772 | B | B |
| I-773 | A | B |
| I-774 | B | B |
| I-775 | A | B |
| I-776 | A | A |
| I-777 | A | B |
| I-778 | B | B |
| I-779 | A | A |
| I-780 | A | A |
| I-781 | A | A |
| I-782 | A | B |
| I-783 | A | B |
| I-784 | A | B |
| I-785 | A | B |
| I-786 | D | D |
| I-787 | C | C |
| I-788 | A | A |
| I-789 | A | B |
| I-790 | B | A |
| I-791 | A | A |
| I-792 | B | B |
| I-793 | B | B |
| I-794 | A | B |
| I-795 | A | A |
| I-796 | A | B |
| I-797 | B | B |
| I-798 | A | B |
| I-799 | F | F |
| I-800 | A | A |
| I-801 | A | A |
| I-802 | A | A |
| I-803 | E | D |
| I-804 | C | B |
| I-805 | E | D |
| I-806 | B | B |
| I-807 | B | B |
| I-808 | E | D |
| I-809 | B | B |
| I-810 | E | D |
| I-811 | E | D |
| I-812 | D | B |
| I-813 | B | A |
| I-814 | B | C |
| I-815 | E | D |
| I-816 | B | B |
| I-817 | C | B |
| I-818 | B | B |
| I-819 | B | A |
| I-820 | B | B |
| I-821 | B | A |
| I-822 | B | A |
| I-823 | C | C |
| I-824 | B | B |
| I-825 | A | A |
| I-826 | A | A |
| I-827 | A | A |
| I-828 | B | A |
| I-829 | F | E |
| I-830 | E | D |
| I-831 | B | B |
| I-832 | A | A |
| I-833 | A | A |
| I-834 | B | A |
| I-835 | A | A |
| I-836 | C | C |
| I-837 | C | A |
| I-838 | C | B |
| I-839 | C | B |
| I-840 | D | B |
| I-841 | B | A |
| I-842 | C | B |
| I-843 | C | B |
| I-844 | B | C |
| I-845 | A | A |
| I-846 | A | A |
| I-847 | A | A |
| I-848 | B | B |
| I-849 | B | A |
| I-850 | B | B |
| I-851 | A | A |
| I-852 | B | B |
| I-853 | A | A |
| I-854 | B | B |
| I-855 | A | A |
| I-856 | B | B |
| I-857 | A | A |
| I-858 | A | A |
| I-859 | B | B |
| I-860 | B | B |
| I-861 | B | C |
| I-862 | C | B |
| I-863 | C | B |
| I-864 | C | B |
| I-865 | C | B |
| I-866 | B | B |
| I-867 | C | B |
| I-868 | B | B |
| I-869 | A | A |
| I-870 | B | B |
| I-871 | B | A |
| I-872 | B | A |
| I-873 | A | A |
| I-874 | A | A |
| I-875 | B | B |
| I-876 | A | A |
| I-877 | A | B |
| I-878 | B | B |
| I-879 | B | A |
| I-880 | B | B |
| I-881 | B | A |
| I-882 | B | B |
| I-883 | C | D |
| I-884 | C | B |
| I-885 | C | B |
| I-886 | A | A |
| I-887 | B | B |
| I-888 | B | B |
| I-889 | A | A |
| I-890 | A | A |
| I-891 | E | D |
| I-892 | C | D |
| I-893 | A | A |
| I-894 | C | D |
| I-895 | B | B |
| I-896 | B | B |
| I-897 | B | B |
| I-898 | A | A |
| I-899 | B | B |
| I-900 | B | B |
| I-901 | B | B |
| I-902 | F | E |
| I-903 | A | A |
| I-904 | B | B |
| I-905 | B | B |
| I-906 | A | B |
| I-907 | A | B |
| I-908 | A | A |
| I-909 | E | D |
| I-910 | A | A |
| I-911 | A | B |
| I-912 | B | B |
| I-913 | B | B |
| I-914 | A | A |
| I-915 | B | B |
| I-916 | E | D |
| I-917 | E | D |
| I-918 | C | C |
| I-919 | F | E |
| I-920 | F | E |
| I-921 | A | A |
| I-922 | C | B |
| I-923 | C | B |
| I-924 | B | B |
| I-925 | A | B |
| I-926 | B | B |
| I-927 | B | C |
| I-928 | B | B |
| I-929 | A | B |
| I-930 | E | D |
| I-931 | F | E |
| I-932 | B | A |
| I-933 | C | C |
| I-934 | E | D |
| I-935 | B | B |
| I-936 | B | B |
| I-937 | B | B |
| I-938 | B | A |
| I-939 | A | A |
| I-940 | B | B |
| I-941 | A | B |
| I-942 | B | B |
| I-943 | A | A |
| I-944 | A | A |
| I-945 | A | A |
| I-946 | B | A |
| I-947 | B | B |
| I-948 | A | A |
| I-949 | F | E |
| I-950 | B | A |
| I-951 | F | E |
| I-952 | F | E |
| I-953 | F | E |
| I-954 | F | E |
| I-955 | F | E |
| I-956 | F | E |
| I-957 | F | E |
| I-958 | F | E |
| I-959 | F | E |
| I-960 | F | E |
| I-961 | E | D |
| I-962 | A | B |
| I-963 | A | A |
| I-964 | C | D |
| I-965 | B | B |
| I-966 | B | B |
| I-967 | B | B |
| I-968 | B | A |
| I-969 | B | A |
| I-970 | A | A |
| I-971 | B | B |
| I-972 | A | A |
| I-973 | B | A |
| I-974 | E | D |
| I-975 | A | A |
| I-976 | B | B |
| I-977 | B | B |
| I-978 | B | A |
| I-979 | E | D |
| I-980 | E | D |
| I-981 | A | A |
| I-982 | B | A |
| I-983 | C | C |
| I-984 | A | B |
| I-985 | B | A |
| I-986 | B | B |
| I-987 | B | A |
| I-988 | A | A |
| I-989 | B | A |
| I-990 | B | B |
| I-991 | A | A |
| I-992 | B | B |
| I-993 | F | E |
| I-994 | F | E |
| I-995 | F | E |
| I-996 | F | E |
| I-997 | F | E |
| I-998 | F | E |
| I-999 | A | A |
| I-1000 | F | E |
| I-1001 | F | E |
| I-1002 | A | A |
| I-1003 | F | E |
| I-1004 | F | E |
| I-1005 | F | E |
| I-1006 | F | E |
| I-1007 | F | E |
| I-1008 | B | A |
| I-1009 | F | E |
| I-1010 | B | A |
| I-1011 | A | A |
| I-1012 | A | A |
| I-1013 | A | B |
| I-1014 | A | B |
| I-1015 | A | B |
| I-1016 | A | A |
| I-1017 | B | A |
| I-1018 | F | E |
| I-1019 | F | E |
| I-1020 | F | E |
| I-1021 | F | E |
| I-1022 | F | E |
| I-1023 | F | E |
| I-1024 | F | E |
| I-1025 | F | E |
| I-1026 | F | E |
| I-1027 | F | E |
| I-1028 | F | E |
| I-1029 | F | E |
| I-1030 | F | E |
| I-1031 | F | E |
| I-1032 | F | E |
| I-1033 | F | E |
| I-1034 | F | E |
| I-1035 | F | E |
| I-1036 | F | E |
| I-1037 | F | E |
| I-1038 | F | E |
| I-1039 | F | E |
| I-1040 | F | E |
| I-1041 | F | E |
| I-1042 | F | E |
| I-1043 | F | E |
| I-1044 | F | E |
| I-1045 | F | E |
| I-1046 | F | E |
| I-1047 | F | E |
| I-1048 | F | E |
| I-1049 | F | E |
| I-1050 | F | E |
| I-1051 | F | E |
| I-1052 | F | E |
| I-1053 | F | E |
| I-1054 | F | E |
| I-1055 | F | E |
| I-1056 | F | E |
| I-1057 | F | E |
| I-1058 | F | E |
| I-1059 | F | E |
| I-1060 | F | E |
| I-1061 | F | E |
| I-1062 | F | E |
| I-1063 | F | E |
| I-1064 | F | E |
| I-1065 | F | E |
| I-1066 | F | E |
| I-1067 | F | E |
| I-1068 | F | E |
| I-1069 | F | E |
| I-1070 | F | E |
| I-1071 | F | E |
| I-1072 | F | E |
| I-1073 | D | B |
| I-1074 | C | B |
| I-1075 | C | B |
| I-1076 | C | C |
| I-1077 | C | B |
| I-1078 | F | E |
| I-1079 | F | E |
| I-1080 | B | A |
| I-1081 | B | A |
| I-1082 | B | A |
| I-1083 | B | A |
| I-1084 | A | A |
| I-1085 | A | A |
| I-1086 | A | A |
| I-1087 | A | A |
| I-1088 | A | A |
| I-1089 | A | A |
| I-1090 | A | A |
| I-1091 | F | E |
| I-1092 | F | E |
| I-1093 | F | E |
| I-1094 | B | A |
| I-1095 | A | A |
| I-1096 | B | A |
| I-1097 | B | B |
| I-1098 | B | B |
| I-1099 | B | B |
| I-1100 | A | B |
| I-1101 | A | A |
| I-1102 | A | A |
| I-1103 | A | A |
| I-1104 | B | A |
| I-1105 | B | A |
| I-1106 | A | A |
| I-1107 | A | A |
| I-1108 | F | E |
| I-1109 | F | E |
| I-1110 | F | E |
| I-1111 | F | E |
| I-1112 | F | E |
| I-1113 | F | E |
| I-1114 | F | E |
| I-1115 | F | E |
| I-1116 | F | E |
| I-1117 | F | E |
| I-1118 | F | E |
| I-1119 | F | E |
| I-1120 | F | E |
| I-1121 | F | E |
| I-1122 | F | E |
| I-1123 | F | E |
| I-1124 | F | E |
| I-1125 | F | E |
| I-1126 | F | E |
| I-1127 | F | E |
| I-1128 | F | E |
| I-1129 | F | E |
| I-1130 | F | E |
| I-1131 | F | E |
| I-1132 | F | E |
| I-1133 | F | E |
| I-1134 | F | E |
| I-1135 | F | E |
| I-1136 | F | E |
| I-1137 | F | E |
| I-1138 | F | E |
| I-1139 | F | E |
| I-1140 | F | E |
| I-1141 | F | E |
| I-1142 | F | E |
| I-1143 | F | E |
| I-1144 | F | E |
| I-1145 | F | E |
| I-1146 | F | E |
| I-1147 | F | E |
| I-1148 | F | E |
| I-1149 | F | E |
| I-1150 | F | E |
| I-1151 | F | E |
| I-1152 | F | E |
| I-1153 | F | E |
| I-1154 | F | E |
| I-1155 | F | E |
| I-1156 | F | E |
| I-1157 | F | E |
| I-1158 | F | E |
| I-1159 | F | E |
| I-1160 | F | E |
| I-1161 | F | E |
| I-1162 | F | E |
| I-1163 | F | E |
| I-1164 | F | E |
| I-1165 | F | E |
| I-1166 | F | E |
| I-1167 | F | E |
| I-1168 | F | E |
| I-1169 | F | E |
| I-1170 | F | E |
| I-1171 | F | E |
| I-1172 | F | E |
| I-1173 | F | E |
| I-1174 | F | E |
| I-1175 | F | E |
| I-1176 | F | E |
| I-1177 | F | E |
| I-1178 | F | E |
| I-1179 | F | E |
| I-1180 | F | E |
| I-1181 | F | E |
| I-1182 | A | B |
| I-1183 | A | B |
| I-1184 | A | B |
| I-1185 | B | A |
| I-1186 | F | E |
| I-1187 | F | E |
| I-1188 | F | E |
| I-1189 | F | E |
| I-1190 | F | E |
| I-1191 | F | E |
| I-1192 | F | E |
| I-1193 | F | E |
| I-1194 | F | E |
| I-1195 | F | E |
| I-1196 | F | E |
| I-1197 | F | E |
| I-1198 | F | E |
| I-1199 | F | E |
| I-1200 | F | E |
| I-1201 | F | E |
| I-1202 | F | E |
| I-1203 | F | E |
| I-1204 | F | E |
| I-1205 | F | E |
| I-1206 | F | E |
| I-1207 | F | E |
| I-1208 | F | E |
| I-1209 | F | E |
| I-1210 | F | E |
| I-1211 | F | E |
| I-1212 | F | E |
| I-1213 | B | D |
| I-1214 | B | B |
| I-1215 | A | B |
| I-1216 | B | B |
| I-1217 | B | C |
| I-1218 | B | B |
| I-1219 | A | B |
| I-1220 | A | B |
| I-1221 | C | B |
| I-1222 | B | B |
| I-1223 | B | B |
| I-1224 | B | B |
| I-1225 | F | E |
| I-1226 | F | E |
| I-1227 | F | E |
| I-1228 | F | E |
| I-1229 | F | E |
| I-1230 | F | E |
| I-1231 | F | E |
| I-1232 | F | E |
| I-1233 | F | E |
| I-1234 | F | E |
| I-1235 | F | E |
| I-1236 | F | E |
| I-1237 | F | E |
| I-1238 | F | E |
| I-1239 | F | E |
| I-1240 | F | E |
| I-1241 | F | E |
| I-1242 | F | E |
| I-1243 | F | E |
| I-1244 | F | E |
| I-1245 | F | E |
| I-1246 | F | E |
| I-1247 | F | E |
| I-1248 | F | E |
| I-1249 | F | E |
| I-1250 | F | E |
| I-1251 | F | E |
| I-1252 | F | E |
| I-1253 | F | E |
| I-1254 | F | E |
| I-1255 | F | E |
| I-1256 | F | E |
| I-1257 | F | E |
| I-1258 | F | E |
| I-1259 | F | E |
| I-1260 | F | E |
| I-1261 | F | E |
| I-1262 | F | E |
| I-1263 | F | E |
| I-1264 | F | E |
| I-1265 | F | E |
| I-1266 | F | E |
| I-1267 | F | E |
| I-1268 | F | E |
| I-1269 | F | E |
| I-1273 | F | E |
| I-1274 | F | E |
| I-1275 | F | E |
| I-1276 | F | E |
| I-1277 | F | E |
| I-1278 | F | E |
| I-1279 | F | E |
| I-1280 | F | E |
| I-1281 | F | E |
| I-1283 | F | E |
| I-1284 | F | E |
| I-1285 | F | E |
| I-1286 | F | E |
| I-1287 | F | E |
| I-1288 | F | E |
| I-1289 | F | E |
| I-1290 | F | E |
| I-1291 | F | E |
| I-1294 | F | E |
| I-1295 | F | E |
| I-1296 | F | E |
| I-1297 | F | E |
| I-1298 | F | E |
| I-1299 | F | E |
| I-1300 | F | E |
| I-1301 | F | E |
| I-1303 | F | E |
| I-1304 | F | E |
| I-1305 | F | E |
| I-1306 | F | E |
| I-1307 | F | E |
| I-1308 | F | E |
| I-1310 | F | E |
| I-1311 | F | E |
| I-1312 | F | E |
| I-1313 | F | E |
| I-1314 | F | E |
| I-1315 | F | E |
| I-1316 | F | E |
| I-1317 | F | E |
| I-1318 | F | E |
| I-1319 | F | E |
| I-1320 | F | E |
| I-1321 | F | E |
| I-1322 | F | E |
| I-1327 | F | E |
| I-1328 | F | E |
| I-1329 | F | E |
| I-1330 | F | E |
| I-1331 | F | E |
| I-1335 | F | E |
| I-1337 | F | E |
| I-1339 | F | E |
| I-1341 | F | E |
| I-1343 | F | E |
| I-1344 | F | E |
| I-1345 | F | E |
| I-1346 | F | E |
| I-1347 | F | E |
| I-1348 | F | E |
| I-1349 | F | E |
| I-1350 | F | E |
| I-1351 | F | E |
| I-1352 | F | E |
| I-1353 | F | E |
| I-1354 | F | E |
| I-1355 | F | E |
| I-1356 | F | E |
| I-1357 | F | E |
| I-1358 | F | E |
| I-1359 | F | E |
| I-1360 | F | E |
| I-1361 | F | E |
| I-1362 | F | E |
| I-1363 | F | E |
| I-1364 | F | E |
| I-1365 | F | E |
| I-1366 | F | E |
| I-1367 | F | E |
| I-1368 | F | E |
| I-1369 | F | E |
| I-1370 | F | E |
| I-1371 | F | E |
| I-1372 | F | E |
| I-1373 | F | E |
| I-1374 | F | E |
| I-1375 | F | E |
| I-1376 | F | E |
| I-1377 | F | E |
| I-1378 | F | E |
| I-1379 | F | E |
| I-1380 | F | E |
| I-1381 | F | E |
| I-1382 | F | E |
| I-1383 | F | E |
| I-1384 | F | E |
| I-1385 | F | E |
| I-1386 | F | E |
| I-1387 | F | E |
| I-1388 | F | E |
| I-1389 | F | E |
| I-1390 | F | E |
| I-1391 | F | E |
| I-1392 | F | E |
| I-1393 | F | E |
| I-1394 | F | E |
| I-1395 | F | E |
| I-1396 | F | E |
| I-1397 | F | E |
| I-1398 | F | E |
| I-1399 | F | E |
| I-1400 | F | E |
| I-1402 | F | E |
| I-1403 | F | E |
| I-1404 | F | E |
| I-1405 | F | E |
| I-1406 | F | E |
| I-1407 | F | E |
| I-1408 | F | E |
| I-1409 | F | F |
| I-1410 | F | E |
| I-1411 | F | E |
| I-1412 | F | E |
| I-1413 | F | E |
| I-1414 | F | E |
| I-1415 | F | E |
| I-1416 | F | E |
| I-1417 | F | E |
| I-1418 | F | E |
| I-1419 | F | E |
| I-1420 | F | E |
| I-1421 | F | E |
| I-1422 | F | E |
| I-1423 | F | E |
| I-1424 | F | E |
| I-1425 | F | E |
| I-1426 | F | E |
| I-1427 | F | E |
| I-1428 | F | E |
| I-1429 | F | E |
| I-1430 | F | E |
| I-1431 | F | E |
| I-1432 | F | E |
| I-1433 | F | E |
| I-1434 | F | E |
| I-1435 | F | E |
| I-1436 | F | E |
| I-1437 | F | E |
| I-1438 | F | E |
| I-1439 | F | E |
| I-1440 | F | E |
| I-1441 | F | E |
| I-1442 | F | E |
| I-1443 | F | E |
| I-1444 | F | E |
| I-1445 | F | E |
| I-1446 | F | E |
| I-1447 | F | E |
| I-1448 | F | E |
| I-1449 | F | E |
| I-1450 | F | E |
| I-1451 | F | E |
| I-1452 | F | E |
| I-1453 | F | E |
| I-1454 | F | E |
| I-1455 | F | E |
| I-1456 | F | E |
| I-1457 | F | E |
| I-1461 | F | E |
| I-1462 | F | E |
| I-1463 | F | E |
| I-1464 | F | E |
| I-1465 | F | E |
| I-1466 | F | E |
| I-1467 | F | E |
| I-1468 | F | E |
| I-1469 | F | E |
| I-1470 | F | E |
| I-1471 | F | E |
| I-1475 | F | E |
| I-1476 | F | E |
| I-1477 | C | C |
| I-1478 | C | B |
| I-1479 | C | B |
| I-1480 | C | B |
| I-1481 | B | A |
| I-1482 | B | B |
| I-1483 | B | B |
| I-1484 | B | B |
| I-1485 | A | B |
| I-1486 | F | E |
| I-1487 | F | E |
| I-1488 | F | E |
| I-1489 | F | E |
| I-1490 | F | E |
| I-1491 | F | E |
| I-1492 | F | E |
| I-1493 | F | E |
| I-1494 | F | E |
| I-1495 | F | E |
| I-1496 | F | E |
| I-1497 | F | E |
| I-1498 | F | E |
| I-1499 | E | D |
| I-1500 | E | D |
| I-1501 | E | D |
| I-1502 | E | D |
| I-1503 | E | D |
| I-1504 | E | D |
| I-1505 | E | D |
| I-1506 | E | D |
| I-1507 | E | D |
| I-1508 | E | D |
| I-1509 | E | D |
| I-1510 | E | D |
| I-1511 | E | D |
| I-1512 | E | D |
| I-1513 | E | D |
| I-1514 | E | D |
| I-1515 | E | D |
| I-1516 | E | D |
| I-1517 | E | D |
| I-1518 | E | D |
| I-1519 | E | D |
| I-1520 | E | D |
| I-1521 | E | D |
| I-1522 | E | D |
| I-1523 | E | D |
| I-1524 | E | D |
| I-1525 | E | D |
| I-1526 | E | D |
| I-1527 | E | D |
| I-1528 | E | D |
| I-1529 | E | D |
| I-1530 | E | D |
| I-1531 | E | D |
| I-1532 | E | D |
| I-1533 | A | A |
| I-1534 | F | E |
| I-1535 | F | E |
| I-1537 | D | D |
| I-1538 | D | D |
| I-1539 | F | E |
| I-1540 | F | E |
| I-1541 | C | B |
| I-1542 | B | D |
| I-1543 | B | B |
| I-1544 | C | C |
| I-1545 | B | B |
| I-1546 | A | A |
| I-1547 | A | A |
| I-1548 | A | A |

### Example 11. STAT6 HiBiT Degradation in A549

Degradation of STAT6 in endogenously tagged STAT6-HiBiT A549 cells was quantitatively measured using the Nano-Glo HiBiT Lytic Detection System (Promega, 3040). A549 (STAT6-HiBiT) cells were seeded in 384-well plates with a density of 1.25k cells per well in 20 μl fresh media. Compounds were then added to the assay plates using the Apricot S3 with a final top concentration of up to 10 uM in a 4-fold dilution series with total of 10 doses. The assay plates were then incubated for 24 hours at 37° C. under 5% CO₂. At the assay endpoint, 20 μL of Nano-Glo HiBiT Lytic Reagent was added to each well and agitated on a plate shaker at 300 rpm for 10 minutes at room temperature. Luminescence was read on PHERAStar FSX Instrument (Is integration time). The data was analyzed by GraphPad Prism and the dose dependent STAT6 degradation was fit using the four-parameter inhibitor vs. response nonlinear regression.

The STAT6 MSD degradation results in A549 cells are shown in Table 16. The letter codes for DC₅₀ (nM) include: A (<1 nM): B (1-10 nM); C (>10-100 nM); D (>100-1000 nM); E (>1000 nM); and F (not tested). The letter codes for Dmax (%) include: A (>90%); B (>70-90%); C (>50-70%); D (<50%); and E (not tested).

TABLE 16
STAT6 HiBiT Degradation Results

| | STAT6 | |
|---|---|---|
| | HiBiT | |
| | degradation | STAT6 HiBiT |
| | DC50 (nM) | degradation |
| I-# | 24 h | Dmax % 24 h |
| I-1 | F | A |
| I-2 | A | A |
| I-3 | F | E |
| I-4 | F | E |
| I-5 | F | E |
| I-6 | F | E |
| I-7 | F | E |
| I-8 | F | E |
| I-9 | F | E |
| I-10 | F | E |
| I-11 | F | E |
| I-12 | F | E |
| I-13 | F | E |
| I-14 | F | E |
| I-15 | F | E |
| I-16 | F | E |
| I-17 | F | E |
| I-18 | F | E |
| I-19 | F | E |
| I-20 | F | E |
| I-21 | F | E |
| I-22 | F | F |
| I-23 | F | E |
| I-24 | F | E |
| I-25 | F | E |
| I-26 | F | E |
| I-27 | F | E |
| I-28 | F | E |
| I-29 | F | E |
| I-30 | F | E |
| I-31 | F | E |
| I-32 | F | E |
| I-33 | F | E |
| I-34 | F | E |
| I-35 | F | E |
| I-36 | F | E |
| I-37 | F | E |
| I-38 | F | E |
| I-39 | F | E |
| I-40 | F | E |
| I-41 | F | E |
| I-42 | F | E |
| I-43 | F | E |
| I-44 | F | E |
| I-45 | F | E |
| I-46 | F | E |
| I-47 | F | E |
| I-48 | F | E |
| I-49 | F | E |
| I-50 | F | E |
| I-51 | F | E |
| I-52 | F | E |
| I-53 | F | E |
| I-54 | F | E |
| I-55 | F | E |
| I-56 | F | E |
| I-57 | F | E |
| I-58 | F | E |
| I-59 | F | E |
| I-60 | F | E |
| I-62 | F | E |
| I-63 | F | E |
| I-64 | F | E |
| I-65 | F | E |
| I-66 | F | E |
| I-67 | F | E |
| I-68 | F | E |
| I-69 | F | E |
| I-70 | F | E |
| I-71 | F | E |
| I-72 | F | E |
| I-73 | F | E |
| I-74 | F | E |
| I-75 | F | E |
| I-76 | F | E |
| I-77 | F | E |
| I-78 | F | E |
| I-79 | F | E |
| I-80 | F | E |
| I-81 | F | E |
| I-82 | F | E |
| I-83 | F | E |
| I-84 | F | E |
| I-85 | F | E |
| I-86 | F | E |
| I-87 | F | E |
| I-88 | F | E |
| I-89 | F | E |
| I-90 | F | E |
| I-91 | F | E |
| I-92 | F | E |
| I-93 | F | E |
| I-94 | F | E |
| I-95 | F | E |
| I-96 | F | E |
| I-97 | F | E |
| I-99 | F | E |
| I-100 | F | E |
| I-101 | F | E |
| I-102 | F | E |
| I-103 | F | E |
| I-104 | F | E |
| I-106 | F | E |
| I-107 | F | E |
| I-108 | F | E |
| I-109 | F | E |
| I-110 | F | E |
| I-111 | F | E |
| I-112 | F | E |
| I-113 | F | E |
| I-114 | F | E |
| I-115 | F | E |
| I-116 | F | E |
| I-117 | F | E |
| I-118 | F | E |
| I-119 | F | E |
| I-120 | F | E |
| I-121 | F | E |
| I-122 | F | E |
| I-123 | F | E |
| I-124 | F | E |
| I-125 | F | E |
| I-126 | F | E |
| I-127 | F | E |
| I-128 | F | E |
| I-129 | F | E |
| I-130 | F | E |
| I-131 | F | E |
| I-132 | F | E |
| I-133 | F | E |
| I-134 | F | E |
| I-135 | F | E |
| I-136 | F | E |
| I-137 | F | E |
| I-138 | F | E |
| I-139 | F | E |
| I-140 | F | E |
| I-141 | F | E |
| I-142 | F | E |
| I-143 | F | E |
| I-144 | F | E |
| I-145 | F | E |
| I-146 | F | E |
| I-147 | F | E |
| I-148 | F | E |
| I-149 | F | E |
| I-150 | F | E |
| I-151 | F | E |
| I-152 | F | E |
| I-153 | F | E |
| I-154 | F | E |
| I-155 | F | E |
| I-156 | F | E |
| I-157 | F | E |
| I-158 | F | E |
| I-159 | F | E |
| I-160 | F | E |
| I-161 | F | E |
| I-162 | F | E |
| I-163 | F | E |
| I-164 | F | E |
| I-165 | F | E |
| I-166 | F | E |
| I-167 | F | E |
| I-168 | F | E |
| I-169 | F | E |
| I-170 | F | E |
| I-171 | F | E |
| I-172 | F | E |
| I-173 | F | E |
| I-174 | F | E |
| I-175 | F | E |
| I-176 | F | E |
| I-177 | F | E |
| I-178 | F | B |
| I-179 | F | E |
| I-180 | F | E |
| I-181 | F | E |
| I-182 | F | E |
| I-183 | F | E |
| I-184 | F | E |
| I-185 | F | E |
| I-186 | F | E |
| I-187 | F | E |
| I-188 | F | E |
| I-189 | F | A |
| I-190 | F | E |
| I-191 | F | E |
| I-192 | F | E |
| I-193 | F | E |
| I-194 | F | E |
| I-195 | F | E |
| I-196 | F | E |
| I-197 | F | E |
| I-198 | F | E |
| I-199 | F | E |
| I-200 | F | E |
| I-201 | F | E |
| I-202 | F | E |
| I-203 | F | E |
| I-204 | | |
| I-205 | F | E |
| I-206 | F | E |
| I-207 | F | E |
| I-208 | F | E |
| I-209 | F | E |
| I-210 | F | E |
| I-211 | F | E |
| I-212 | F | E |
| I-213 | F | E |
| I-214 | F | E |
| I-215 | F | E |
| I-216 | F | E |
| I-217 | F | E |
| I-218 | F | E |
| I-219 | F | E |
| I-220 | F | E |
| I-221 | F | E |
| I-222 | F | E |
| I-223 | F | E |
| I-224 | F | E |
| I-225 | F | E |
| I-226 | F | E |
| I-227 | F | E |
| I-228 | F | E |
| I-229 | F | E |
| I-230 | F | E |
| I-231 | F | E |
| I-232 | F | E |
| I-233 | F | E |
| I-234 | F | E |
| I-235 | F | E |
| I-236 | F | E |
| I-237 | F | E |
| I-238 | F | E |
| I-239 | F | E |
| I-240 | F | E |
| I-241 | F | E |
| I-242 | B | A |
| I-243 | F | E |
| I-244 | F | E |
| I-245 | F | E |
| I-246 | F | E |
| I-247 | F | E |
| I-248 | F | E |
| I-249 | F | E |
| I-250 | F | E |
| I-251 | F | E |
| I-252 | F | E |
| I-253 | F | E |
| I-254 | F | E |
| I-255 | B | A |
| I-256 | F | E |
| I-257 | F | E |
| I-258 | F | E |
| I-259 | F | E |
| I-260 | F | E |
| I-261 | F | E |
| I-262 | F | E |
| I-263 | F | A |
| I-264 | F | E |
| I-265 | F | E |
| I-266 | F | E |
| I-267 | F | E |
| I-268 | F | E |
| I-269 | F | E |
| I-270 | F | E |
| I-271 | F | E |
| I-272 | F | E |
| I-273 | F | E |
| I-274 | F | E |
| I-275 | F | E |
| I-276 | F | E |
| I-277 | F | E |
| I-278 | F | E |
| I-279 | F | E |
| I-280 | F | E |
| I-281 | F | E |
| I-282 | F | E |
| I-283 | F | E |
| I-284 | F | E |
| I-285 | F | E |
| I-286 | F | E |
| I-287 | F | E |
| I-288 | F | E |
| I-289 | F | E |
| I-290 | F | E |
| I-291 | F | E |
| I-292 | F | E |
| I-293 | F | E |
| I-294 | F | E |
| I-295 | F | E |
| I-296 | F | E |
| I-297 | F | E |
| I-298 | F | E |
| I-299 | F | E |
| I-300 | F | E |
| I-301 | F | E |
| I-302 | E | D |
| I-303 | F | E |
| I-304 | F | E |
| I-305 | F | E |
| I-306 | F | E |
| I-307 | F | E |
| I-308 | F | E |
| I-309 | F | E |
| I-310 | F | E |
| I-311 | F | E |
| I-312 | F | E |
| I-313 | F | E |
| I-314 | F | E |
| I-315 | F | E |
| I-316 | F | E |
| I-317 | F | E |
| I-318 | F | E |
| I-319 | F | E |
| I-320 | F | E |
| I-321 | F | E |
| I-322 | F | E |
| I-323 | F | E |
| I-324 | F | E |
| I-325 | F | E |
| I-326 | F | E |
| I-327 | F | E |
| I-328 | F | E |
| I-329 | F | E |
| I-330 | F | E |
| I-331 | F | E |
| I-332 | F | E |
| I-333 | F | E |
| I-334 | F | E |
| I-335 | F | E |
| I-336 | F | A |
| I-337 | F | E |
| I-338 | F | E |
| I-339 | F | E |
| I-340 | F | E |
| I-341 | F | E |
| I-342 | F | E |
| I-343 | F | E |
| I-344 | F | E |
| I-345 | F | E |
| I-346 | F | E |
| I-347 | F | E |
| I-348 | F | E |
| I-349 | F | E |
| I-350 | F | E |
| I-351 | F | E |
| I-352 | F | E |
| I-353 | F | E |
| I-354 | F | E |
| I-355 | F | E |
| I-356 | F | E |
| I-357 | F | E |
| I-358 | F | E |
| I-359 | F | E |
| I-360 | F | E |
| I-361 | F | E |
| I-362 | F | E |
| I-363 | F | E |
| I-364 | F | E |
| I-365 | F | E |
| I-366 | F | E |
| I-367 | F | E |
| I-368 | F | E |
| I-369 | F | E |
| I-370 | F | E |
| I-371 | F | E |
| I-372 | F | E |
| I-373 | F | E |
| I-374 | F | E |
| I-375 | F | E |
| I-376 | F | E |
| I-377 | F | E |
| I-378 | F | E |
| I-379 | F | E |
| I-380 | F | E |
| I-381 | F | E |
| I-382 | F | E |
| I-383 | F | E |
| I-384 | F | E |
| I-385 | F | E |
| I-386 | F | E |
| I-387 | F | E |
| I-388 | F | E |
| I-389 | F | E |
| I-390 | F | E |
| I-391 | F | E |
| I-392 | F | E |
| I-393 | F | E |
| I-394 | F | E |
| I-395 | F | E |
| I-396 | F | E |
| I-397 | F | E |
| I-398 | F | E |
| I-399 | F | E |
| I-400 | F | E |
| I-401 | F | E |
| I-402 | F | E |
| I-403 | F | E |
| I-404 | F | E |
| I-405 | F | E |
| I-406 | F | E |
| I-407 | F | E |
| I-408 | F | E |
| I-409 | F | E |
| I-410 | F | E |
| I-411 | F | E |
| I-412 | F | E |
| I-413 | F | E |
| I-414 | F | E |
| I-415 | F | E |
| I-416 | F | E |
| I-417 | F | E |
| I-418 | F | E |
| I-419 | F | E |
| I-420 | F | E |
| I-421 | F | E |
| I-422 | F | E |
| I-423 | F | E |
| I-424 | F | E |
| I-425 | F | E |
| I-426 | F | E |
| I-427 | F | E |
| I-428 | F | E |
| I-429 | F | E |
| I-430 | F | E |
| I-431 | F | E |
| I-432 | F | E |
| I-433 | F | E |
| I-434 | F | E |
| I-435 | F | E |
| I-436 | F | E |
| I-437 | F | E |
| I-438 | F | E |
| I-439 | F | E |
| I-440 | F | E |
| I-441 | F | E |
| I-442 | F | E |
| I-443 | F | E |
| I-444 | F | E |
| I-445 | F | E |
| I-446 | F | E |
| I-447 | F | E |
| I-448 | F | E |
| I-449 | F | E |
| I-450 | F | E |
| I-451 | F | E |
| I-452 | F | E |
| I-453 | F | E |
| I-454 | F | E |
| I-455 | F | E |
| I-456 | F | E |
| I-457 | F | E |
| I-458 | F | E |
| I-459 | F | E |
| I-460 | F | E |
| I-461 | F | E |
| I-462 | F | E |
| I-463 | F | E |
| I-464 | F | E |
| I-465 | F | E |
| I-466 | F | E |
| I-467 | F | E |
| I-468 | F | E |
| I-469 | F | E |
| I-470 | F | E |
| I-471 | F | E |
| I-472 | F | E |
| I-473 | F | E |
| I-474 | F | E |
| I-475 | F | E |
| I-476 | F | E |
| I-477 | F | E |
| I-478 | F | E |
| I-479 | F | E |
| I-480 | F | E |
| I-481 | F | E |
| I-482 | F | E |
| I-483 | F | E |
| I-484 | F | E |
| I-485 | F | E |
| I-486 | F | E |
| I-487 | F | E |
| I-488 | F | E |
| I-489 | F | E |
| I-490 | F | E |
| I-491 | F | E |
| I-492 | F | E |
| I-493 | F | E |
| I-494 | F | E |
| I-495 | F | E |
| I-496 | F | E |
| I-497 | F | E |
| I-498 | F | E |
| I-499 | F | E |
| I-500 | F | E |
| I-501 | F | E |
| I-502 | F | E |
| I-503 | F | E |
| I-504 | F | E |
| I-505 | F | E |
| I-506 | F | E |
| I-507 | F | E |
| I-508 | F | E |
| I-509 | F | E |
| I-510 | F | E |
| I-511 | F | E |
| I-512 | F | E |
| I-513 | F | E |
| I-514 | F | E |
| I-515 | F | E |
| I-516 | F | E |
| I-517 | F | E |
| I-518 | F | E |
| I-519 | F | E |
| I-520 | F | E |
| I-521 | F | E |
| I-522 | F | E |
| I-523 | F | E |
| I-524 | F | E |
| I-525 | F | E |
| I-526 | F | E |
| I-527 | F | E |
| I-528 | F | E |
| I-529 | F | E |
| I-530 | F | E |
| I-531 | F | E |
| I-532 | F | E |
| I-533 | F | E |
| I-534 | F | E |
| I-535 | F | E |
| I-536 | F | E |
| I-537 | F | E |
| I-538 | F | E |
| I-539 | F | E |
| I-540 | F | E |
| I-541 | F | E |
| I-542 | F | E |
| I-543 | F | E |
| I-544 | F | B |
| I-545 | F | E |
| I-546 | F | E |
| I-547 | F | E |
| I-548 | F | E |
| I-549 | F | E |
| I-550 | F | E |
| I-551 | F | E |
| I-552 | F | E |
| I-553 | F | E |
| I-554 | F | E |
| I-555 | F | E |
| I-556 | F | E |
| I-557 | F | E |
| I-558 | F | E |
| I-559 | F | E |
| I-560 | F | E |
| I-561 | F | E |
| I-562 | F | E |
| I-563 | F | E |
| I-564 | F | E |
| I-565 | F | E |
| I-566 | F | E |
| I-567 | F | E |
| I-568 | F | E |
| I-569 | F | E |
| I-570 | F | E |
| I-571 | F | E |
| I-572 | F | E |
| I-573 | F | E |
| I-574 | F | E |
| I-575 | F | E |
| I-576 | F | E |
| I-577 | F | E |
| I-578 | F | E |
| I-579 | F | E |
| I-580 | F | E |
| I-581 | F | E |
| I-582 | F | E |
| I-583 | F | E |
| I-584 | F | E |
| I-585 | F | E |
| I-586 | F | E |
| I-587 | F | E |
| I-588 | F | E |
| I-589 | F | E |
| I-590 | F | E |
| I-591 | F | E |
| I-592 | F | E |
| I-593 | F | E |
| I-594 | F | E |
| I-595 | F | E |
| I-596 | F | E |
| I-597 | F | E |
| I-598 | F | E |
| I-599 | F | E |
| I-600 | F | E |
| I-601 | F | E |
| I-602 | F | E |
| I-603 | F | E |
| I-604 | F | E |
| I-605 | F | E |
| I-606 | F | E |
| I-607 | F | E |
| I-608 | F | E |
| I-609 | F | E |
| I-610 | F | E |
| I-611 | F | E |
| I-612 | F | E |
| I-613 | F | E |
| I-614 | F | E |
| I-615 | F | E |
| I-616 | F | E |
| I-617 | F | E |
| I-618 | F | E |
| I-619 | F | E |
| I-620 | F | E |
| I-621 | F | E |
| I-622 | F | E |
| I-623 | F | E |
| I-624 | F | E |
| I-625 | F | E |
| I-626 | F | E |
| I-627 | F | E |
| I-628 | F | A |
| I-629 | F | E |
| I-630 | F | E |
| I-631 | F | E |
| I-632 | F | E |
| I-633 | F | E |
| I-634 | F | E |
| I-635 | F | E |
| I-636 | F | E |
| I-637 | F | E |
| I-638 | F | E |
| I-639 | F | E |
| I-640 | F | E |
| I-641 | F | E |
| I-642 | F | E |
| I-643 | F | E |
| I-644 | F | E |
| I-645 | F | E |
| I-646 | F | E |
| I-647 | F | E |
| I-648 | F | E |
| I-649 | F | E |
| I-650 | F | E |
| I-651 | F | E |
| I-652 | F | E |
| I-653 | F | E |
| I-654 | F | E |
| I-655 | F | E |
| I-656 | F | E |
| I-657 | F | E |
| I-658 | F | E |
| I-659 | F | E |
| I-660 | F | E |
| I-661 | F | E |
| I-662 | F | E |
| I-663 | F | E |
| I-664 | F | E |
| I-665 | F | E |
| I-666 | F | E |
| I-667 | F | E |
| I-668 | F | E |
| I-669 | F | E |
| I-670 | F | E |
| I-671 | F | E |
| I-672 | F | E |
| I-673 | F | E |
| I-674 | F | E |
| I-675 | F | E |
| I-676 | F | E |
| I-677 | A | A |
| I-678 | F | E |
| I-679 | F | E |
| I-680 | F | E |
| I-681 | F | E |
| I-682 | F | E |
| I-683 | F | E |
| I-684 | F | E |
| I-685 | F | E |
| I-686 | F | E |
| I-687 | F | E |
| I-688 | F | E |
| I-689 | F | E |
| I-690 | F | E |
| I-691 | F | E |
| I-692 | A | A |
| I-693 | F | E |
| I-694 | F | E |
| I-695 | F | E |
| I-696 | F | E |
| I-697 | F | E |
| I-698 | F | E |
| I-699 | F | E |
| I-700 | F | E |
| I-701 | F | E |
| I-702 | F | E |
| I-703 | F | E |
| I-704 | F | E |
| I-705 | F | E |
| I-706 | F | E |
| I-707 | F | E |
| I-708 | F | E |
| I-709 | F | E |
| I-710 | F | E |
| I-711 | F | E |
| I-712 | F | E |
| I-713 | F | E |
| I-714 | F | E |
| I-715 | F | E |
| I-716 | F | E |
| I-717 | F | E |
| I-718 | F | E |
| I-719 | F | E |
| I-720 | F | E |
| I-721 | F | E |
| I-722 | F | E |
| I-723 | F | E |
| I-724 | F | E |
| I-725 | F | E |
| I-726 | A | A |
| I-727 | F | E |
| I-728 | A | B |
| I-729 | A | A |
| I-730 | A | A |
| I-731 | F | E |
| I-732 | F | E |
| I-733 | F | A |
| I-734 | F | A |
| I-735 | F | E |
| I-736 | F | B |
| I-737 | F | E |
| I-738 | F | E |
| I-739 | F | E |
| I-740 | A | A |
| I-741 | A | A |
| I-742 | F | E |
| I-743 | F | E |
| I-744 | F | E |
| I-745 | F | E |
| I-746 | A | A |
| I-747 | F | E |
| I-748 | F | E |
| I-749 | F | E |
| I-750 | F | E |
| I-751 | F | E |
| I-752 | F | E |
| I-753 | F | E |
| I-754 | F | E |
| I-755 | F | E |
| I-756 | F | E |
| I-757 | F | A |
| I-758 | F | E |
| I-759 | A | A |
| I-760 | F | E |
| I-761 | F | E |
| I-762 | F | E |
| I-763 | F | E |
| I-764 | F | E |
| I-765 | F | E |
| I-766 | F | E |
| I-767 | F | E |
| I-768 | F | E |
| I-769 | F | E |
| I-770 | F | E |
| I-771 | F | E |
| I-772 | F | E |
| I-773 | F | E |
| I-774 | F | E |
| I-775 | F | E |
| I-776 | F | E |
| I-777 | F | E |
| I-778 | F | E |
| I-779 | F | E |
| I-780 | F | E |
| I-781 | F | E |
| I-782 | F | E |
| I-783 | F | E |
| I-784 | F | E |
| I-785 | F | E |
| I-786 | F | E |
| I-787 | F | E |
| I-788 | F | E |
| I-789 | F | E |
| I-790 | F | E |
| I-791 | F | E |
| I-792 | F | E |
| I-793 | F | E |
| I-794 | F | E |
| I-795 | F | E |
| I-796 | F | E |
| I-797 | F | E |
| I-798 | F | E |
| I-799 | F | E |
| I-800 | F | A |
| I-801 | F | A |
| I-802 | F | E |
| I-803 | F | E |
| I-804 | F | E |
| I-805 | F | E |
| I-806 | F | E |
| I-807 | F | E |
| I-808 | F | E |
| I-809 | F | E |
| I-810 | F | E |
| I-811 | F | E |
| I-812 | F | E |
| I-813 | F | E |
| I-814 | F | E |
| I-815 | F | E |
| I-816 | F | E |
| I-817 | F | E |
| I-818 | F | E |
| I-819 | F | E |
| I-820 | F | E |
| I-821 | F | E |
| I-822 | F | E |
| I-823 | F | E |
| I-824 | F | E |
| I-825 | F | E |
| I-826 | F | E |
| I-827 | F | E |
| I-828 | F | E |
| I-829 | F | E |
| I-830 | F | E |
| I-831 | F | E |
| I-832 | F | E |
| I-833 | F | E |
| I-834 | F | E |
| I-835 | F | E |
| I-836 | F | E |
| I-837 | F | E |
| I-838 | F | E |
| I-839 | F | E |
| I-840 | F | E |
| I-841 | F | E |
| I-842 | F | E |
| I-843 | F | E |
| I-844 | F | E |
| I-845 | F | E |
| I-846 | F | E |
| I-847 | F | E |
| I-848 | F | E |
| I-849 | F | E |
| I-850 | F | E |
| I-851 | F | E |
| I-852 | F | E |
| I-853 | F | E |
| I-854 | F | E |
| I-855 | F | E |
| I-856 | F | E |
| I-857 | F | E |
| I-858 | A | A |
| I-859 | F | E |
| I-860 | F | E |
| I-861 | F | E |
| I-862 | F | E |
| I-863 | F | E |
| I-864 | F | E |
| I-865 | F | E |
| I-866 | F | E |
| I-867 | F | E |
| I-868 | F | E |
| I-869 | F | E |
| I-870 | F | E |
| I-871 | F | E |
| I-872 | F | E |
| I-873 | F | E |
| I-874 | F | E |
| I-875 | F | E |
| I-876 | F | E |
| I-877 | F | E |
| I-878 | F | E |
| I-879 | F | E |
| I-880 | F | E |
| I-881 | F | E |
| I-882 | F | E |
| I-883 | F | E |
| I-884 | F | E |
| I-885 | F | E |
| I-886 | F | E |
| I-887 | F | E |
| I-888 | F | E |
| I-889 | F | E |
| I-890 | F | E |
| I-891 | F | E |
| I-892 | F | E |
| I-893 | F | E |
| I-894 | F | E |
| I-895 | F | E |
| I-896 | F | E |
| I-897 | F | E |
| I-898 | F | E |
| I-899 | F | E |
| I-900 | F | E |
| I-901 | F | E |
| I-902 | F | E |
| I-903 | F | E |
| I-904 | F | E |
| I-905 | F | E |
| I-906 | F | E |
| I-907 | F | E |
| I-908 | F | E |
| I-909 | F | E |
| I-910 | F | E |
| I-911 | F | E |
| I-912 | F | E |
| I-913 | F | E |
| I-914 | F | E |
| I-915 | F | E |
| I-916 | F | E |
| I-917 | F | E |
| I-918 | F | E |
| I-919 | F | E |
| I-920 | F | E |
| I-921 | F | E |
| I-922 | F | E |
| I-923 | F | E |
| I-924 | F | E |
| I-925 | F | E |
| I-926 | F | E |
| I-927 | F | E |
| I-928 | F | E |
| I-929 | F | E |
| I-930 | F | E |
| I-931 | F | E |
| I-932 | F | E |
| I-933 | F | E |
| I-934 | F | E |
| I-935 | F | E |
| I-936 | F | E |
| I-937 | F | E |
| I-938 | F | E |
| I-939 | F | E |
| I-940 | F | E |
| I-941 | F | E |
| I-942 | F | E |
| I-943 | F | E |
| I-944 | F | E |
| I-945 | F | E |
| I-946 | F | E |
| I-947 | F | E |
| I-948 | F | A |
| I-949 | F | E |
| I-950 | F | E |
| I-951 | F | E |
| I-952 | F | E |
| I-953 | F | E |
| I-954 | F | E |
| I-955 | F | E |
| I-956 | F | E |
| I-957 | F | E |
| I-958 | F | E |
| I-959 | F | E |
| I-960 | F | E |
| I-961 | F | E |
| I-962 | F | E |
| I-963 | F | A |
| I-964 | F | E |
| I-965 | F | E |
| I-966 | F | E |
| I-967 | F | E |
| I-968 | A | A |
| I-969 | F | E |
| I-970 | F | E |
| I-971 | F | E |
| I-972 | F | E |
| I-973 | F | E |
| I-974 | F | E |
| I-975 | F | E |
| I-976 | F | E |
| I-977 | F | E |
| I-978 | B | A |
| I-979 | F | E |
| I-980 | F | E |
| I-981 | F | E |
| I-982 | F | E |
| I-983 | F | E |
| I-984 | F | E |
| I-985 | F | E |
| I-986 | F | E |
| I-987 | F | E |
| I-988 | F | E |
| I-989 | F | E |
| I-990 | F | E |
| I-991 | F | E |
| I-992 | F | E |
| I-993 | F | E |
| I-994 | F | E |
| I-995 | F | E |
| I-996 | F | E |
| I-997 | F | E |
| I-998 | F | E |
| I-999 | F | E |
| I-1000 | F | E |
| I-1001 | F | E |
| I-1002 | A | A |
| I-1003 | A | A |
| I-1004 | A | A |
| I-1005 | A | A |
| I-1006 | A | A |
| I-1007 | F | E |
| I-1008 | F | E |
| I-1009 | F | E |
| I-1010 | F | E |
| I-1011 | F | E |
| I-1012 | A | A |
| I-1013 | A | A |
| I-1014 | A | A |
| I-1015 | A | A |
| I-1016 | A | A |
| I-1017 | A | A |
| I-1018 | A | A |
| I-1019 | A | A |
| I-1020 | A | A |
| I-1021 | A | A |
| I-1022 | A | A |
| I-1023 | A | A |
| I-1024 | A | A |
| I-1025 | A | A |
| I-1026 | A | A |
| I-1027 | A | A |
| I-1028 | A | A |
| I-1029 | A | A |
| I-1030 | A | A |
| I-1031 | A | A |
| I-1032 | A | A |
| I-1033 | A | A |
| I-1034 | A | A |
| I-1035 | A | A |
| I-1036 | A | A |
| I-1037 | A | A |
| I-1038 | A | A |
| I-1039 | A | A |
| I-1040 | A | A |
| I-1041 | A | A |
| I-1042 | A | A |
| I-1043 | A | A |
| I-1044 | A | A |
| I-1045 | A | A |
| I-1046 | A | A |
| I-1047 | A | A |
| I-1048 | A | A |
| I-1049 | F | E |
| I-1050 | F | E |
| I-1051 | F | E |
| I-1052 | F | E |
| I-1053 | F | E |
| I-1054 | F | E |
| I-1055 | F | E |
| I-1056 | F | E |
| I-1057 | F | E |
| I-1058 | F | E |
| I-1059 | F | E |
| I-1060 | F | E |
| I-1061 | F | E |
| I-1062 | F | E |
| I-1063 | F | E |
| I-1064 | F | E |
| I-1065 | F | E |
| I-1066 | F | E |
| I-1067 | F | E |
| I-1068 | F | E |
| I-1069 | F | E |
| I-1070 | F | E |
| I-1071 | F | E |
| I-1072 | F | E |
| I-1073 | F | E |
| I-1074 | F | E |
| I-1075 | F | E |
| I-1076 | F | E |
| I-1077 | F | E |
| I-1078 | A | A |
| I-1079 | A | A |
| I-1080 | F | E |
| I-1081 | F | E |
| I-1082 | F | E |
| I-1083 | F | E |
| I-1084 | F | E |
| I-1085 | F | E |
| I-1086 | F | E |
| I-1087 | A | A |
| I-1088 | A | A |
| I-1089 | A | A |
| I-1090 | A | A |
| I-1091 | A | A |
| I-1092 | A | A |
| I-1093 | B | B |
| I-1094 | F | E |
| I-1095 | F | E |
| I-1096 | F | E |
| I-1097 | F | E |
| I-1098 | F | E |
| I-1099 | F | E |
| I-1100 | A | A |
| I-1101 | A | A |
| I-1102 | A | A |
| I-1103 | A | A |
| I-1104 | B | A |
| I-1105 | B | A |
| I-1106 | A | A |
| I-1107 | A | A |
| I-1108 | A | A |
| I-1109 | A | A |
| I-1110 | A | A |
| I-1111 | A | B |
| I-1112 | B | A |
| I-1113 | A | A |
| I-1114 | B | A |
| I-1115 | B | B |
| I-1116 | B | A |
| I-1117 | A | A |
| I-1118 | A | A |
| I-1119 | A | A |
| I-1120 | A | B |
| I-1121 | A | A |
| I-1122 | A | A |
| I-1123 | A | A |
| I-1124 | A | A |
| I-1125 | A | B |
| I-1126 | A | A |
| I-1127 | A | A |
| I-1128 | A | A |
| I-1129 | B | A |
| I-1130 | B | A |
| I-1131 | A | A |
| I-1132 | A | B |
| I-1133 | A | B |
| I-1134 | A | A |
| I-1135 | A | A |
| I-1136 | A | A |
| I-1137 | A | A |
| I-1138 | B | A |
| I-1139 | A | A |
| I-1140 | B | A |
| I-1141 | B | A |
| I-1142 | B | A |
| I-1143 | A | A |
| I-1144 | A | A |
| I-1145 | A | A |
| I-1146 | A | A |
| I-1147 | A | A |
| I-1148 | A | A |
| I-1149 | A | A |
| I-1150 | A | A |
| I-1151 | A | A |
| I-1152 | B | A |
| I-1153 | A | A |
| I-1154 | A | A |
| I-1155 | A | A |
| I-1156 | A | A |
| I-1157 | A | A |
| I-1158 | A | A |
| I-1159 | A | A |
| I-1160 | A | A |
| I-1161 | A | A |
| I-1162 | A | A |
| I-1163 | A | A |
| I-1164 | A | A |
| I-1165 | A | A |
| I-1166 | A | A |
| I-1167 | A | A |
| I-1168 | A | A |
| I-1169 | A | A |
| I-1170 | A | A |
| I-1171 | A | A |
| I-1172 | A | A |
| I-1173 | A | A |
| I-1174 | B | C |
| I-1175 | A | A |
| I-1176 | A | B |
| I-1177 | A | A |
| I-1178 | B | A |
| I-1179 | A | A |
| I-1180 | B | A |
| I-1181 | A | A |
| I-1182 | A | A |
| I-1183 | A | A |
| I-1184 | A | A |
| I-1185 | B | A |
| I-1186 | A | A |
| I-1187 | A | A |
| I-1188 | A | A |
| I-1189 | A | A |
| I-1190 | A | A |
| I-1191 | A | A |
| I-1192 | A | B |
| I-1193 | A | A |
| I-1194 | A | A |
| I-1195 | A | A |
| I-1196 | A | A |
| I-1197 | A | A |
| I-1198 | A | A |
| I-1199 | A | A |
| I-1200 | B | D |
| I-1201 | A | A |
| I-1202 | A | A |
| I-1203 | B | B |
| I-1204 | A | A |
| I-1205 | A | A |
| I-1206 | A | A |
| I-1207 | A | A |
| I-1208 | A | B |
| I-1209 | A | A |
| I-1210 | A | A |
| I-1211 | A | A |
| I-1212 | B | A |
| I-1213 | F | E |
| I-1214 | F | E |
| I-1215 | F | E |
| I-1216 | F | E |
| I-1217 | F | E |
| I-1218 | F | E |
| I-1219 | F | E |
| I-1220 | F | E |
| I-1221 | B | B |
| I-1222 | B | B |
| I-1223 | B | B |
| I-1224 | B | B |
| I-1225 | B | B |
| I-1226 | B | C |
| I-1227 | B | B |
| I-1228 | C | C |
| I-1229 | C | B |
| I-1230 | B | B |
| I-1231 | B | B |
| I-1232 | B | B |
| I-1233 | B | B |
| I-1234 | A | B |
| I-1235 | A | D |
| I-1236 | A | B |
| I-1237 | A | B |
| I-1238 | B | D |
| I-1239 | B | B |
| I-1240 | B | B |
| I-1241 | A | C |
| I-1242 | B | D |
| I-1243 | B | D |
| I-1244 | B | B |
| I-1245 | A | B |
| I-1246 | A | B |
| I-1247 | B | B |
| I-1248 | A | B |
| I-1249 | B | B |
| I-1250 | B | B |
| I-1251 | B | B |
| I-1252 | B | B |
| I-1253 | A | B |
| I-1254 | B | B |
| I-1255 | B | D |
| I-1256 | C | D |
| I-1257 | A | B |
| I-1258 | B | B |
| I-1259 | A | C |
| I-1260 | B | C |
| I-1261 | B | B |
| I-1262 | A | B |
| I-1263 | B | B |
| I-1264 | B | C |
| I-1265 | B | D |
| I-1266 | A | A |
| I-1267 | B | D |
| I-1268 | A | A |
| I-1269 | C | C |
| I-1273 | F | E |
| I-1274 | F | E |
| I-1275 | F | E |
| I-1276 | A | A |
| I-1277 | A | A |
| I-1278 | A | A |
| I-1279 | A | A |
| I-1280 | F | E |
| I-1281 | F | E |
| I-1283 | A | A |
| I-1284 | A | A |
| I-1285 | B | A |
| I-1286 | B | A |
| I-1287 | A | A |
| I-1288 | E | D |
| I-1289 | E | D |
| I-1290 | A | A |
| I-1291 | F | E |
| I-1294 | F | E |
| I-1295 | A | A |
| I-1296 | A | A |
| I-1297 | A | A |
| I-1298 | A | A |
| I-1299 | A | A |
| I-1300 | B | B |
| I-1301 | A | A |
| I-1303 | A | A |
| I-1304 | F | E |
| I-1305 | F | E |
| I-1306 | A | A |
| I-1307 | B | A |
| I-1308 | B | A |
| I-1310 | F | E |
| I-1311 | A | A |
| I-1312 | F | E |
| I-1313 | A | A |
| I-1314 | A | A |
| I-1315 | A | A |
| I-1316 | A | A |
| I-1317 | A | A |
| I-1318 | A | A |
| I-1319 | A | A |
| I-1320 | F | E |
| I-1321 | F | E |
| I-1322 | B | B |
| I-1327 | C | B |
| I-1328 | E | D |
| I-1329 | F | E |
| I-1330 | F | E |
| I-1331 | F | E |
| I-1335 | A | A |
| I-1337 | C | C |
| I-1339 | E | D |
| I-1341 | E | D |
| I-1343 | | |
| I-1344 | F | E |
| I-1345 | F | E |
| I-1346 | F | E |
| I-1347 | F | E |
| I-1348 | A | A |
| I-1349 | F | E |
| I-1350 | F | E |
| I-1351 | F | E |
| I-1352 | F | E |
| I-1353 | A | A |
| I-1354 | F | E |
| I-1355 | F | E |
| I-1356 | A | A |
| I-1357 | A | A |
| I-1358 | A | A |
| I-1359 | F | E |
| I-1360 | F | E |
| I-1361 | A | A |
| I-1362 | F | E |
| I-1363 | F | E |
| I-1364 | B | A |
| I-1365 | F | E |
| I-1366 | F | E |
| I-1367 | F | E |
| I-1368 | F | E |
| I-1369 | F | E |
| I-1370 | A | A |
| I-1371 | F | E |
| I-1372 | F | E |
| I-1373 | A | A |
| I-1374 | A | A |
| I-1375 | A | A |
| I-1376 | A | A |
| I-1377 | A | A |
| I-1378 | | |
| I-1379 | A | A |
| I-1380 | A | A |
| I-1381 | B | C |
| I-1382 | F | E |
| I-1383 | F | E |
| I-1384 | F | E |
| I-1385 | F | E |
| I-1386 | F | E |
| I-1387 | F | E |
| I-1388 | A | A |
| I-1389 | A | A |
| I-1390 | A | A |
| I-1391 | F | E |
| I-1392 | B | C |
| I-1393 | F | E |
| I-1394 | A | D |
| I-1395 | A | A |
| I-1396 | E | D |
| I-1397 | A | A |
| I-1398 | F | E |
| I-1399 | A | B |
| I-1400 | F | E |
| I-1402 | E | D |
| I-1403 | F | E |
| I-1404 | B | D |
| I-1405 | E | D |
| I-1406 | A | A |
| I-1407 | F | E |
| I-1408 | A | A |
| I-1409 | A | A |
| I-1410 | A | A |
| I-1411 | D | C |
| I-1412 | B | B |
| I-1413 | F | E |
| I-1414 | F | E |
| I-1415 | F | E |
| I-1416 | A | A |
| I-1417 | A | A |
| I-1418 | A | A |
| I-1419 | B | B |
| I-1420 | B | B |
| I-1421 | A | B |
| I-1422 | A | A |
| I-1423 | A | A |
| I-1424 | A | A |
| I-1425 | A | A |
| I-1426 | A | B |
| I-1427 | B | C |
| I-1428 | C | B |
| I-1429 | B | A |
| I-1430 | C | B |
| I-1431 | A | A |
| I-1432 | B | B |
| I-1433 | C | B |
| I-1434 | A | A |
| I-1435 | A | A |
| I-1436 | A | A |
| I-1437 | B | A |
| I-1438 | A | A |
| I-1439 | A | A |
| I-1440 | A | A |
| I-1441 | A | A |
| I-1442 | A | A |
| I-1443 | A | A |
| I-1444 | A | A |
| I-1445 | A | A |
| I-1446 | A | A |
| I-1447 | A | A |
| I-1448 | B | A |
| I-1449 | B | A |
| I-1450 | A | A |
| I-1451 | A | A |
| I-1452 | A | A |
| I-1453 | B | A |
| I-1454 | B | B |
| I-1455 | A | B |
| I-1456 | C | B |
| I-1457 | C | C |
| I-1461 | C | D |
| I-1462 | D | B |
| I-1463 | D | C |
| I-1464 | C | B |
| I-1465 | F | E |
| I-1466 | A | A |
| I-1467 | F | E |
| I-1468 | B | A |
| I-1469 | F | E |
| I-1470 | F | E |
| I-1471 | F | E |
| I-1475 | A | A |
| I-1476 | F | E |
| I-1477 | F | E |
| I-1478 | F | E |
| I-1479 | F | E |
| I-1480 | F | E |
| I-1481 | F | E |
| I-1482 | F | E |
| I-1483 | F | E |
| I-1484 | F | E |
| I-1485 | F | E |
| I-1486 | B | A |
| I-1487 | A | A |
| I-1488 | A | B |
| I-1489 | A | A |
| I-1490 | A | A |
| I-1491 | A | B |
| I-1492 | F | E |
| I-1493 | F | E |
| I-1494 | F | E |
| I-1495 | A | A |
| I-1496 | B | B |
| I-1497 | A | B |
| I-1498 | A | B |
| I-1499 | F | E |
| I-1500 | F | E |
| I-1501 | F | E |
| I-1502 | F | E |
| I-1503 | F | E |
| I-1504 | F | E |
| I-1505 | F | E |
| I-1506 | F | E |
| I-1507 | F | E |
| I-1508 | F | E |
| I-1509 | F | E |
| I-1510 | F | E |
| I-1511 | F | E |
| I-1512 | F | E |
| I-1513 | F | E |
| I-1514 | E | D |
| I-1515 | E | D |
| I-1516 | E | D |
| I-1517 | E | D |
| I-1518 | E | D |
| I-1519 | E | D |
| I-1520 | E | D |
| I-1521 | E | D |
| I-1522 | E | D |
| I-1523 | E | D |
| I-1524 | E | D |
| I-1525 | E | D |
| I-1526 | E | D |
| I-1527 | E | D |
| I-1528 | E | D |
| I-1529 | E | D |
| I-1530 | E | D |
| I-1531 | E | D |
| I-1532 | E | D |
| I-1533 | A | A |
| I-1534 | F | E |
| I-1535 | F | E |
| I-1537 | F | E |
| I-1538 | F | E |
| I-1539 | F | E |
| I-1540 | F | E |
| I-1541 | F | E |
| I-1542 | F | E |
| I-1543 | F | E |
| I-1544 | F | E |
| I-1545 | F | E |
| I-1546 | F | E |
| I-1547 | F | E |
| I-1548 | F | E |

### EXEMPLARY ENUMERATED EMBODIMENTS

1. A compound of formula I-bb-1:

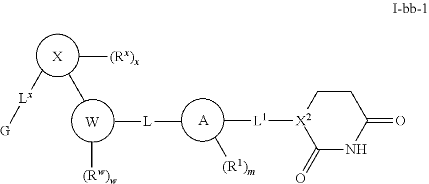

or a pharmaceutically acceptable salt thereof, wherein:
Ring W is a 9-membered bicyclic heteroaryl with 1-4 heteroatoms independently selected from nitrogen, oxygen, and sulfur;
Ring X is a 6-membered saturated or partially unsaturated carbocyclylenyl or heterocyclylenyl with 1-3 heteroatoms independently selected from nitrogen, oxygen, and sulfur;
G is hydrogen, halogen, or

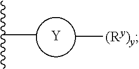

Ring Y is a 3- to 6-membered saturated or partially unsaturated carbocyclyl, or 4- to 6-membered monocyclic saturated or partially unsaturated heterocyclyl or heteroaryl ring with 1-4 heteroatoms independently selected from nitrogen, oxygen, or sulfur;
R^{w}, R^{x}, and R^{y} are independently selected from hydrogen, R^{A}, halogen, —CN, —NO₂, —OR, —SR, —NR₂, —SiR₃, —S(O)R, —S(O)₂R, —S(O)(NR)R, —S(O)₂NR₂, —S(O)R, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R;
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-10 membered monocyclic or bicyclic heteroaryl ring having 1-4 heteroatoms independently selected from nitrogen, oxygen, and sulfur;
L^{X} is a covalent bond or a C₁₋₅ bivalent straight or branched saturated or unsaturated hydrocarbon chain wherein 1-2 methylene units of the chain are independently and optionally replaced with —O—, —C(O)—, —C(S)—, —CR₂—, —NR—, —S—, —S(O)—, or —S(O)₂—;
each of w, x, and y are independently 0, 1, 2, 3, or 4;
L is a covalent bond, -Cy-(CH₂)₁₋₁₀, —(CH₂)₁₋₁₀-Cy-(CH₂)₁₋₁₀—, —(CH₂)₁₋₁₀-Cy-(CH₂CH₂O)₁₋₁₀CH₂CH₂—, -Cy-(CH₂)₁₋₁₀-Cy-, -Cy-(CH₂)₁₋₁₀-Cy-(CH₂)₁₋₁—, -Cy-(CH₂)₁₋₁₀-Cy-(CH₂)₁₋₁₀-Cy-, —(CH₂)₁₋₁₀-Cy-(CH₂)₁₋₁₀-Cy-(CH₂)₁₋₁₀—, -Cy-Cy-, -Cy-Cy-(CH₂)₁₋₁₀—, -Cy-(CH₂)₁₋₁₀-Cy-(CH₂)₁₋₁₀—, -Cy-Cy-Cy-, -Cy-Cy-(CH₂)₁₋₁₀-Cy-, or -Cy-Cy-(CH₂)₁₋₁₀-Cy-(CH₂)₁₋₁₀—;
each -Cy- is independently an optionally substituted bivalent ring selected from phenylenyl, an 8-10 membered bicyclic arylenyl, a 4-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur;
X² is N or CH;
each R¹ is independently RA, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)N(R)OR, —C(R)₂N(R)C(O)R, —C(R)₂N(R)C(O)NR₂, —OC(O)R, —OC(O)NR₂, —OP(O)R₂, —OP(O)(OR)₂. —OP(O)(OR)(NR₂), —OP(O)(NR₂)₂, —N(R)C(O)OR, —N(R)C(O)R, —N(R)C(O)NR₂, —N(R)S(O)₂R, —NP(O)R₂, —N(R)P(O)(OR)₂, —N(R)P(O)(OR)(NR₂), —N(R)P(O)(NR₂)₂, —N(R)S(O)₂R;
L¹ is a covalent bond, —C(O)—, —NR—, —O—, —S—, —S(O)₂, —NRC(O)—, or —C(O)NR—; L¹ is a covalent bond, —C(O)—, —NR—, —O—, —S—, —S(O)₂, —NRC(O)—, or —C(O)NR—;
Ring A is phenylenyl or a 5 to 10-membered saturated or partially unsaturated monocyclic or bicyclic heterocyclylenyl or heteroarylenyl having 1-4 heteroatoms independently selected from nitrogen, oxygen, and sulfur;
each R is independently hydrogen, or an optionally substituted group selected from C₁ Å aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same or adjacent atoms or R^{B} and an R group are taken together with their intervening atoms to form an optionally substituted 3-11 membered saturated or partially unsaturated monocyclic, bicyclic, bridged bicyclic, or spirocyclic carbocyclic or heterocyclic ring having 0-3 heteroatoms independently selected from nitrogen, oxygen, and sulfur;
m is 0, 1, 2, 3, 4, or 5.
2. The embodiment of embodiment 1, wherein Ring Y is a 5-membered monocyclic heteroaryl ring with 1-4 heteroatoms independently selected from nitrogen, oxygen, and sulfur.
3. The compound of embodiment 1 or 2, wherein Ring W is:

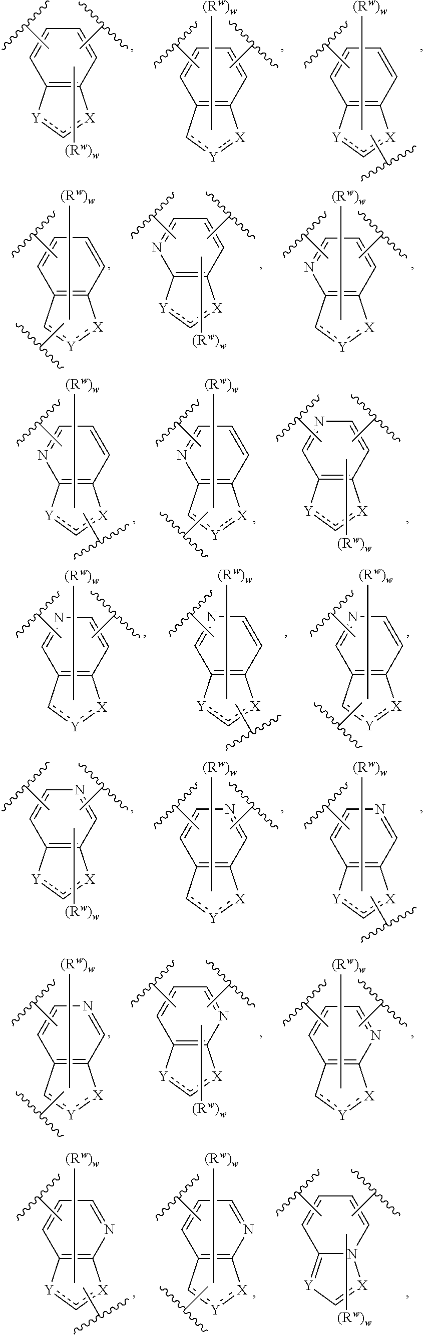

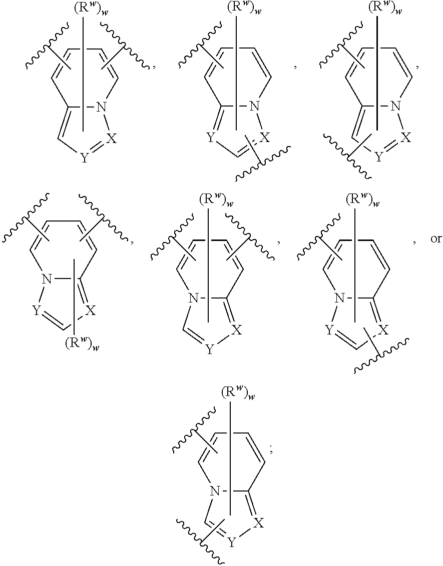

wherein:
each of X and Y is independently N, NH, N—R^{W}, —O—, —S—, C—H, C—R^{W}, C—H₂, CH(R^{W}), or C—(R^{W})₂.
4. The compound of embodiment 3, wherein:
X is C—R^{w} or CH and Y is N—R^{w};
X is C—R^{w} or CH and Y is S;
X is C—R^{w} or CH and Y is O;
X is N—R^{w} or C—H and Y is C—R^{w} or CH;
X is S and Y is C—R^{w} or CH; or
X is O and Y is C—R^{w} or CH.
5. The compound of any one of embodiments 1-4, wherein Ring A is phenylenyl, pyridinylenyl,

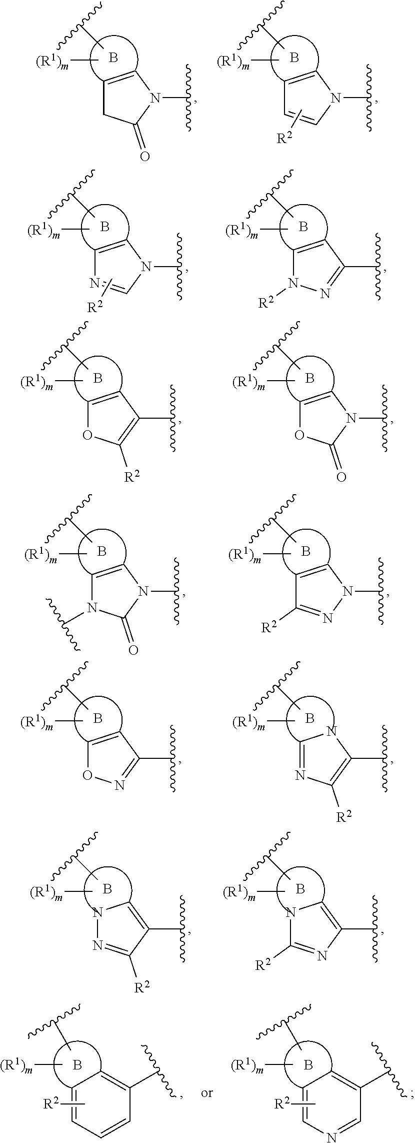

wherein Ring B is a fused ring selected from benzo or a 5-6 membered heteroaryl containing 1-4 heteroatoms independently selected from nitrogen, oxygen, and sulfur; and
R² is hydrogen, halogen, C₁₋₆ alkyl, C₃₋₆cycloalkyl, C₁₋₆ haloalkyl, —OC₁₋₆ alkyl, —OC₃₋₆cycloalkyl, or —OC₁₋₆ haloalkyl
6. The compound of any one of embodiments 1-5, wherein the compound is of formula I-bb-1, I-bb-1-v, I-bb-2, I-bb-2-v, I-bb-3, or I-bb-3-iv:

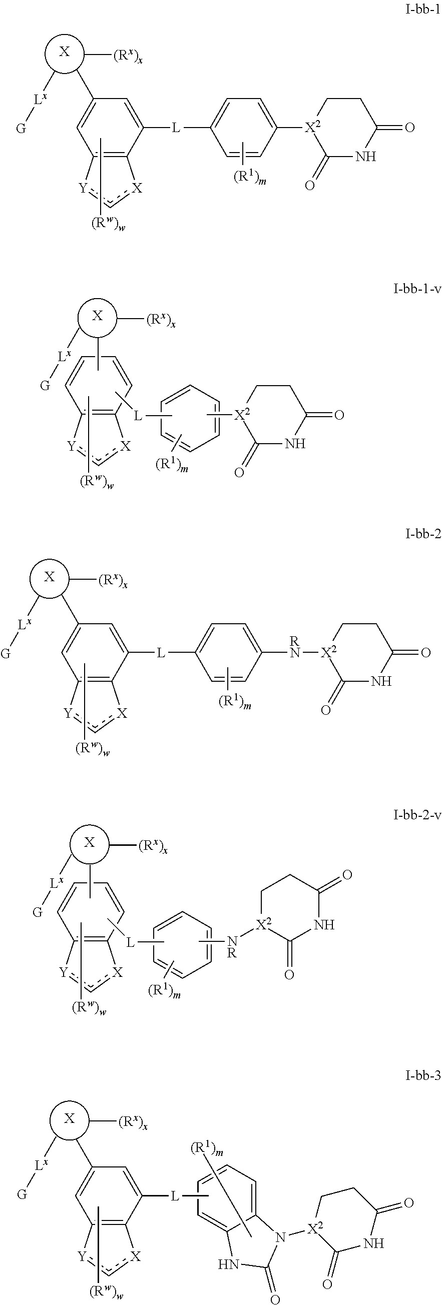

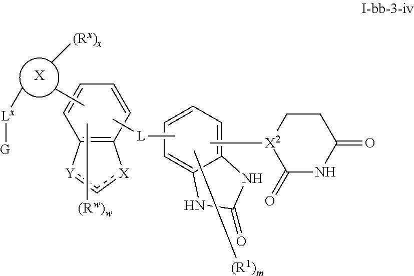

or a pharmaceutically acceptable salt thereof.
7. The compound of any one of embodiments 1-5, wherein the compound is of formula I-bb-1a, 1-bb-1a-v, I-bb-2a, I-bb-2a-v, I-bb-3a, or I-bb-3a-iv:

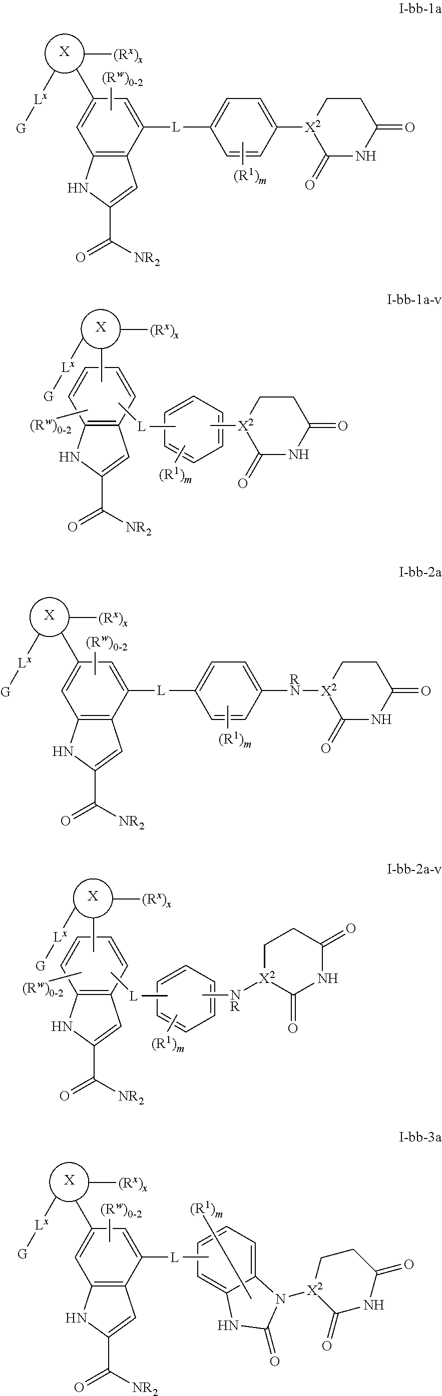

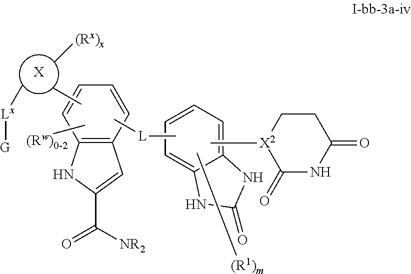

or a pharmaceutically acceptable salt thereof; or
the compound is of formula I-bb-1b, I-bb-1b-iii, I-bb-2b, I-bb-2b-iii, I-bb-3b, or I-bb-3b-iii:

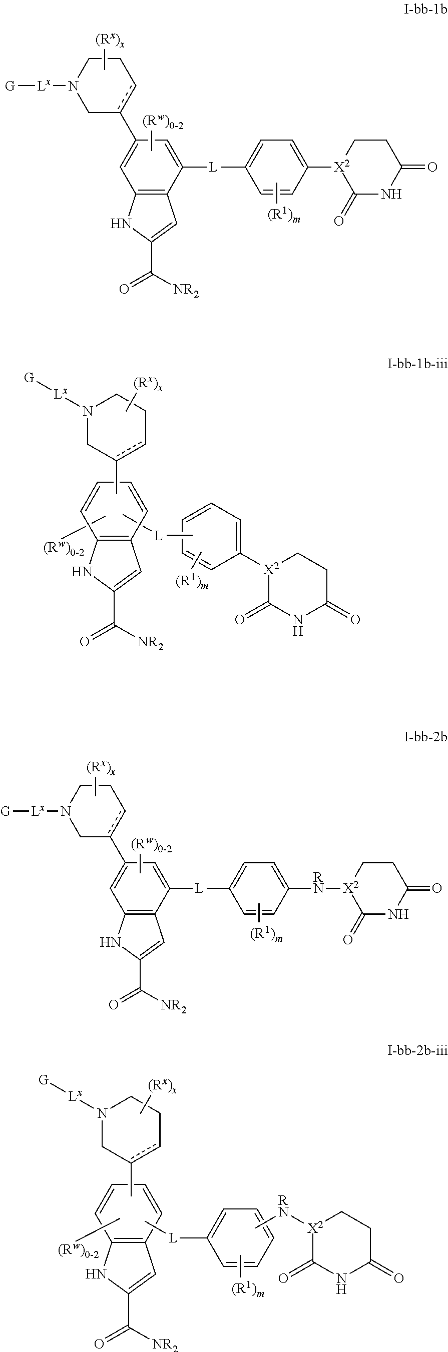

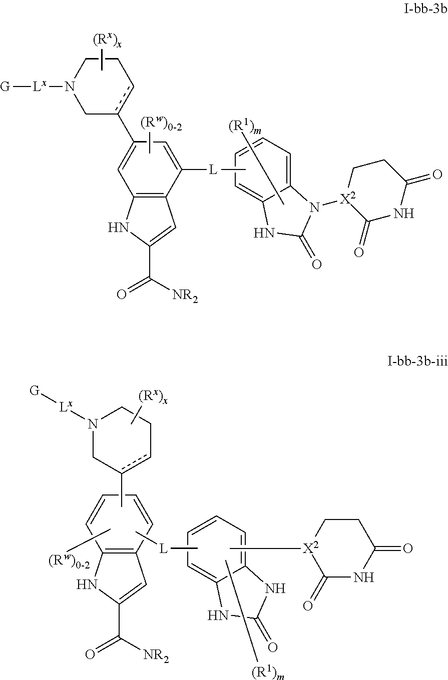

or a pharmaceutically acceptable salt thereof; or
compound is of formula I-bb-lc, I-bb-lc-iii, I-bb-2c. I-bb-2c-iii. I-bb-3c, or I-bb-3c-iii:

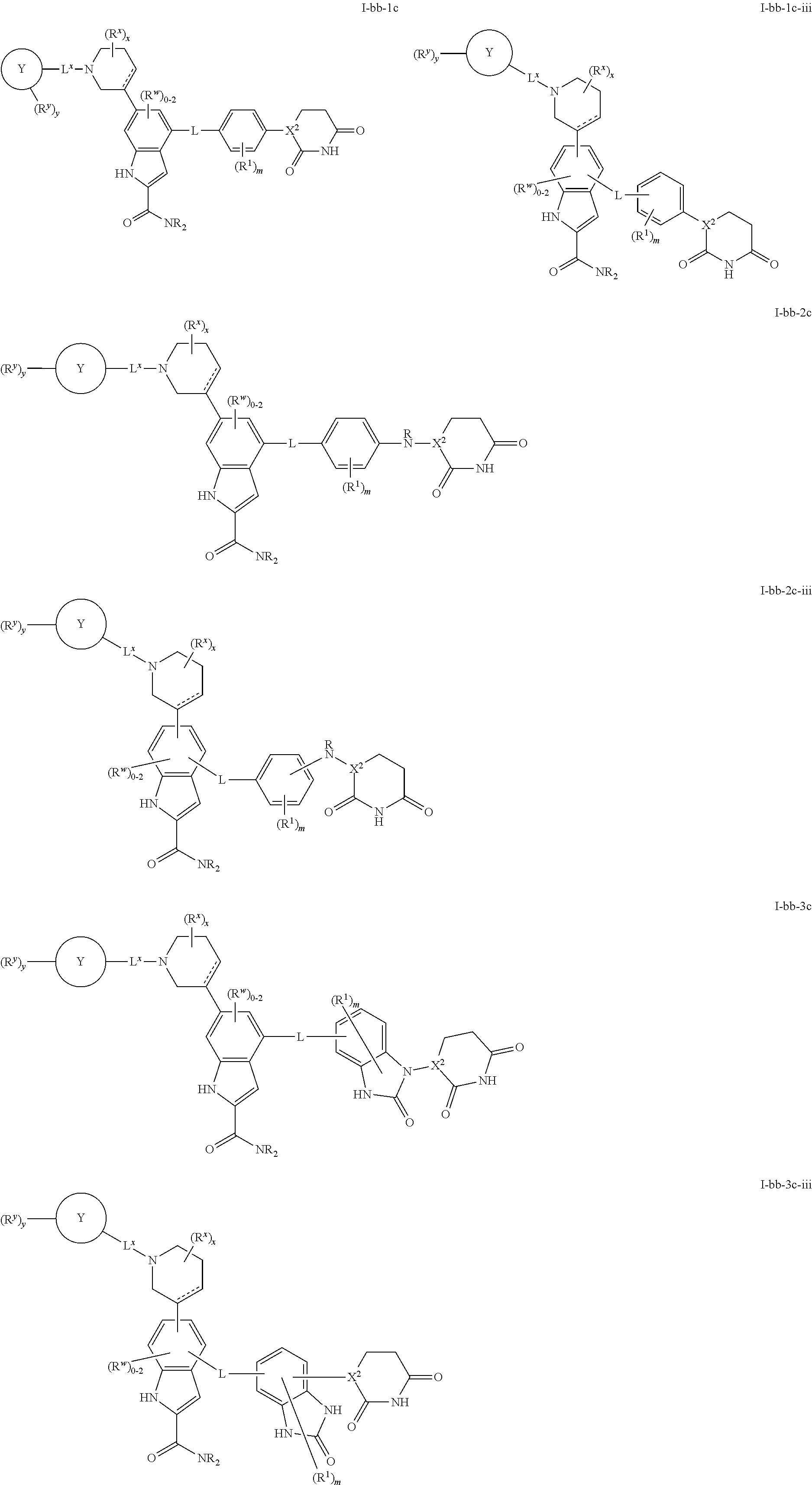

or a pharmaceutically acceptable salt thereof.
8. The compound of any one of embodiments 1-7, wherein Ring Y is triazolyl or pyrazolyl

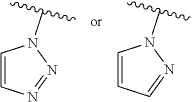 [SMILES: *n1ccnn1 ; *n1cccn1]

9. The compound of any one of embodiments 1-8, wherein Lx is a C₁₋₅ bivalent straight saturated or unsaturated hydrocarbon chain wherein 1-2 methylene units of the chain are independently replaced with —O—, —C(O)—, or —NR—,
10. The compound of any one of embodiments 1-9, where an occurrence of R^{w} is halogen.
11. The compound of any one of embodiments 1-10, wherein R¹ is C₁₋₆ aliphatic, halogen, —CN, —NO₂, —OR, —SR, or —NR₂.
12. The compound of any one of embodiments 1-11, wherein y is 0.
13. A pharmaceutical composition comprising a compound of any one of embodiments 1-12, and a pharmaceutically acceptable carrier, adjuvant, or vehicle.
14. A method of degrading STAT6 in a patient or biological sample comprising administering to said patient, or contacting said biological sample with a compound of any one of embodiments 1-12, or a pharmaceutical composition thereof.
15. A method of treating an STAT6-mediated disorder, disease, or condition in a patient comprising administering to said patient a compound of any of one embodiments 1-12, or a pharmaceutical composition thereof.
16. The method of embodiment 19, wherein STAT6-mediated disorder, disease, or condition is cancer, a neurodegenerative disorder, a viral disease, an autoimmune disease, an inflammatory disorder, a hereditary disorder, a hormone-related disease, a metabolic disorder, conditions associated with organ transplantation, immunodeficiency disorders, a destructive or overgrowing bone disorder, a proliferative disorder, an infectious disease, a condition associated with cell death, thrombin-induced platelet aggregation, liver disease, pathologic immune conditions involving T cell activation, a cardiovascular disorder, or a CNS disorder.
101. A compound is of formula I-bb:

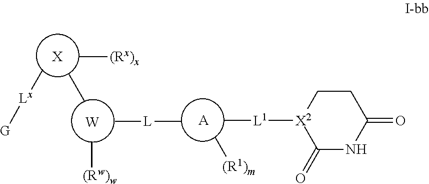

or a pharmaceutically acceptable salt thereof, wherein
Ring W is:

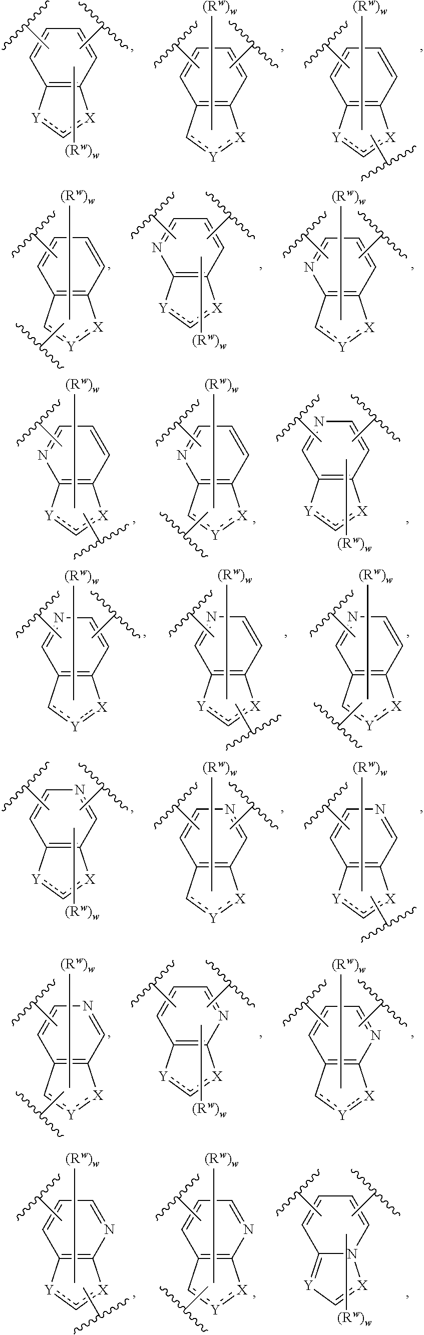

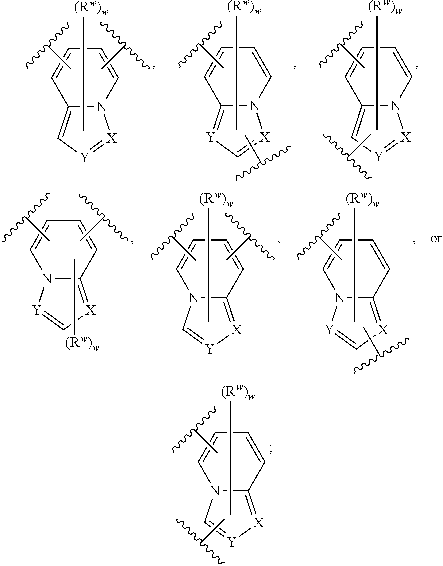

wherein:
each of X and Y is independently N, NH, N—R^{W}, —O—, —S—, C—H, C—R^{W}, CH₂, CH(R^{W}), or C(R^{W})₂, as alloWed by valency;
G is hydrogen, halogen, or

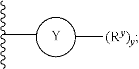

Ring X is

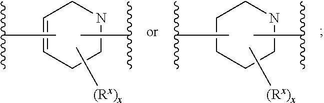

Ring Y is a 5-6 membered monocyclic heteroaryl ring with 1-4 heteroatoms independently selected form nitrogen, oxygen, and sulfur;
wherein L^{X} is a covalent bond or ^{#}-L^{XA}-L^{XB}-, wherein:
   ^{#} represents the point of attachment to Ring X;
   L^{XA} is —C(O)—, —C(S)—, —CR₂—, —CR(OR)—, —C(NR)—, —S(O)—, —S(O)₂—or —S(O)(NR)—; and
   L^{XB} is an optionally substituted C₁₋₄ bivalent straight or branched saturated or unsaturated hydrocarbon chain;
L is a covalent bond optionally substituted bivalent, saturated or partially unsaturated, straight or branched C₁₋₁₀ hydrocarbon chain, wherein 0-6 methylene units of L are independently replaced by -Cy-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—;
X² is N or C—R^{B};
R^{B} is H, halogen or C₁₋₃ alkyl;
L¹ is a covalent bond, —C(O)—, —NR—, —O—, —S—, —S(O)₂, —NRC(O)—, or —C(O)NR—; and
Ring A is phenylenyl, naphthalenyl, a 4-7 membered saturated or partially unsaturated heterocyclylenyl having 1-3 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, an 8-10 membered bicyclic heteroarylenyl having 1-5 heteroatoms independently selected from nitrogen, oxygen, and sulfur, a 12-13 membered saturated or partially unsaturated tricyclic heterocyclylenyl having 1-5 heteroatoms independently selected from nitrogen, oxygen, and sulfur, or a 13 membered tricyclic heteroarylenyl having 1-5 heteroatoms independently selected from nitrogen, oxygen, and sulfur.
102. The compound of embodiment 101, wherein:
L is a covalent bond, -Cy-(CH₂)₁₋₁₀—, —(CH₂)₁₋₁₀-Cy-(CH₂)₁₋₁₀—, —(CH₂)₁₋₁₀-Cy-(CH₂CH₂O)₁₋₁₀CH₂CH₂—, -Cy-(CH₂)₁₋₁₀-Cy-, -Cy-(CH₂)₁₋₁₀-Cy-(CH₂)₁₋₁₀—, -Cy-(CH₂)₁₋₁₀-Cy-(CH₂)₁₋₁₀-Cy-, —(CH₂)₁₋₁₀-Cy-(CH₂)₁₋₁₀-Cy-(CH₂)₁₋₁₀—, -Cy-Cy-, -Cy-Cy-(CH₂)₁₋₁₀—, -Cy-(CH₂)₁₋₁₀-Cy-(CH₂)₁₋₁₀—, -Cy-Cy-Cy-, -Cy-Cy-(CH₂)₁₋₁₀-Cy-, -Cy-Cy-(CH₂)₁₋₁₀-Cy-(CH₂)₁₋₁₀—, —(CH₂)₁₋₁₀-Cy-Cy-(CH₂)₁₋₁₀—, —O-Cy-Cy-(CH₂)₁₋₁₀—, or -Cy-Cy-(CH₂)₁₋₁₀—NR—;
X² is N or C—R^{B} and
   R^{B} is H or C₁₋₃ alkyl.
103. The compound of embodiment 101 or 102, wherein:
X is C—R^{w} or CH, and Y is N—R^{w};
X is C—R^{w} or CH, and Y is S;
X is C—R^{w} or CH, and Y is O;
X is N—R^{w} or NH, and Y is C—R^{w} or CH;
X is S, and Y is C—R^{w} or CH; or
X is O, and Y is C—R^{w} or CH.
104. The compound of any one of embodiments 101-103, wherein Ring Y is a 5 membered monocyclic heteroaryl ring with 1-4 heteroatoms independently selected form nitrogen, oxygen, and sulfur.
105. The compound of any one of embodiments 101-104, wherein Ring Y is pyrazolyl, imidazolyl, or triazolyl.
106. The compound of any one of embodiments 101-105, wherein L^{XA} is —C(O)—,
107. The compound of embodiments 101-106, wherein an occurrence of R^{w} is —C(S)R, —C(NR)R, —S(O)R, —S(O)₂R, —S(O)(NR)R—S(O)₂NR₂, —S(O)R, —C(O)R, —C(O)OR, —C(O)NR₂,—C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, or —NRS(O)₂R,
108. The compound of embodiments 101-107, wherein an occurrence of R^{w} is —C(S)R, —C(NR)R, —C(O)R, —C(O)OR, or —C(O)NR₂.
109. The compound of embodiments 101-108, wherein an occurrence of R^{w} is an optionally substituted 5-6 membered monocyclic heteroaryl ring having 1-4 heteroatoms independently selected from nitrogen, oxygen, and sulfur.
110. The compound of any one of embodiments 101-108, wherein an occurrence of R^{w} is halogen (e.g., fluoro).
111. The compound of any one of embodiments 101-110, wherein v is 0.
112. The compound of any one of embodiments 101-111 wherein the compound is of formula I-bb-I, I-bb-IX, I-bb-A, I-bb-B, I-bb-C. I-bb-D, I-bb-E, or I-bb-F:

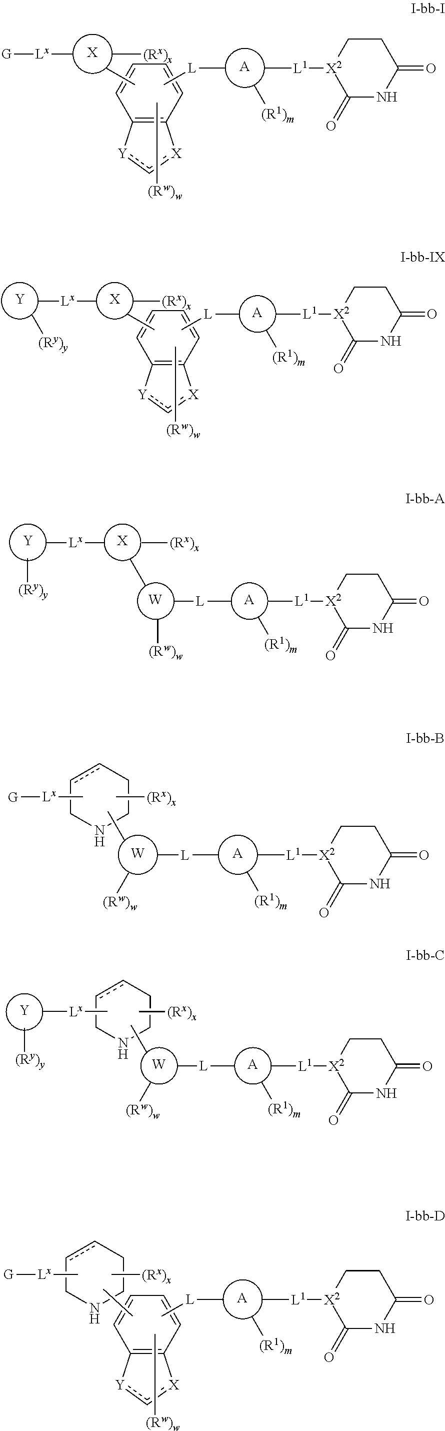

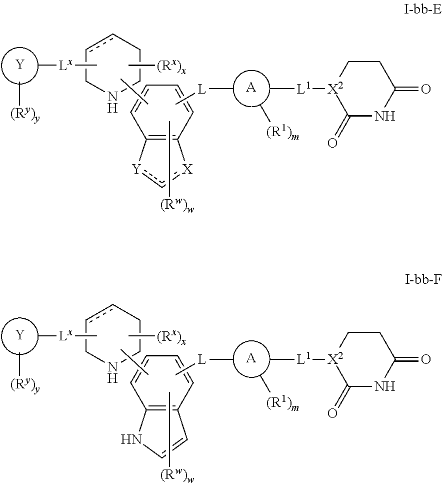

or a pharmaceutically acceptable salt thereof.
113. The compound of any one of embodiments 101-111 wherein the compound is of formula I-cc-1, I-cc-2, or I-cc-3:

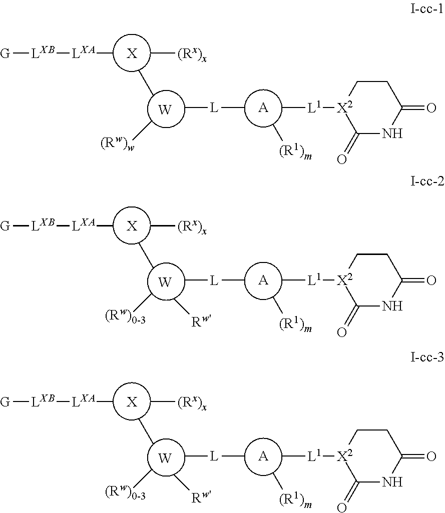

or a pharmaceutically acceptable salt thereof, wherein:
R^{w′} is —S(O)R, —S(O)₂R, —S(O)(NR)R—S(O)₂NR₂, —C(O)R, —C(S)R, —C(NR)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, —NRS(O)₂R, or an optionally substituted group selected from C₁₋₆ aliphatic, phenyl, naphthalenyl, a 3-7 membered saturated or partially unsaturated carbocyclic or heterocyclic ring having 1-2 heteroatoms independently selected from nitrogen, oxygen, and sulfur, and a 5-10 membered monocyclic or bicyclic heteroaryl ring having 1-4 heteroatoms independently selected from nitrogen, oxygen, and sulfur.
114. The compound of embodiment 113, wherein R_{w′} is an optionally substituted 5-6 membered heteroaryl having 1-4 heteroatoms independently selected from nitrogen, oxygen, and sulfur.
115. The compound of embodiment 113, wherein:
R^{w′} is

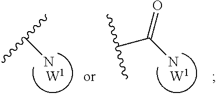

and
Ring W¹ is an optionally substituted 3-7 membered saturated or partially unsaturated heterocyclic ring having 1 additional heteroatom selected from nitrogen, oxygen, and sulfur, or a 5-6 membered monocyclic heteroaryl ring having 1-3 additional heteroatoms independently selected from nitrogen, oxygen, and sulfur.
116. The compound of embodiment 113, wherein:
R^{w′} is

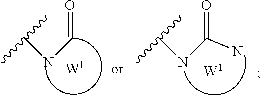

and
Ring W¹ is an optionally substituted 3-7 membered saturated or partially unsaturated heterocyclic ring having 1 additional heteroatom selected from nitrogen, oxygen, and sulfur, or a 5-6 membered monocyclic heteroaryl ring having 1-3 additional heteroatoms independently selected from nitrogen, oxygen, and sulfur.
117. The compound of embodiment 113, wherein:
R^{w′} is

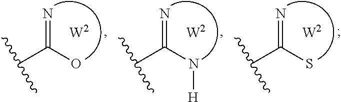

and
Ring W² is an optionally substituted 3-7 membered partially unsaturated heterocyclic ring, or a 5-6 membered monocyclic heteroaryl ring having 1-2 additional heteroatoms independently selected from nitrogen, oxygen, and sulfur.
118. The compound of embodiment 113, wherein R^{w′} is —S(O)R, —S(O)₂R, —S(O)(NR)R, —S(O)₂NR₂, —S(O)R, —C(S)R, —C(NR)R, —C(O)R, —C(O)OR, —C(O)NR₂, or —C(O)NROR,
119. The compound of embodiment 113, embodiment R_{w′} is —S(O)R, —S(O)₂R, —S(O)(NR)R, —S(O)₂NR₂, —S(O)R, —C(S)R, —C(NR)R, —C(O)R, —C(O)OR, —C(O)NR₂, or —C(O)NROR,
120. The compound of embodiment 113, wherein R^{′} is —C(S)R, —C(NR)R, —C(O)R, —C(O)OR, or —C(O)NR₂
121. The compound of any one of embodiments 101-111 or 113-120 wherein the compound is of formula I-cc-1A, I-cc-2A, or I-cc-3A:

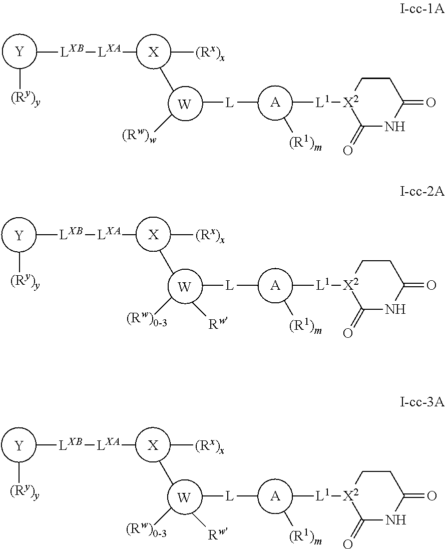

or a pharmaceutically acceptable salt thereof.
121. The compound of any one of embodiments 101-111 or 113-120 wherein the compound is of formula I-cc-1B, I-cc-2B, I-cc-3B, I-cc-IC, I-cc-2C, or I-cc-3C:

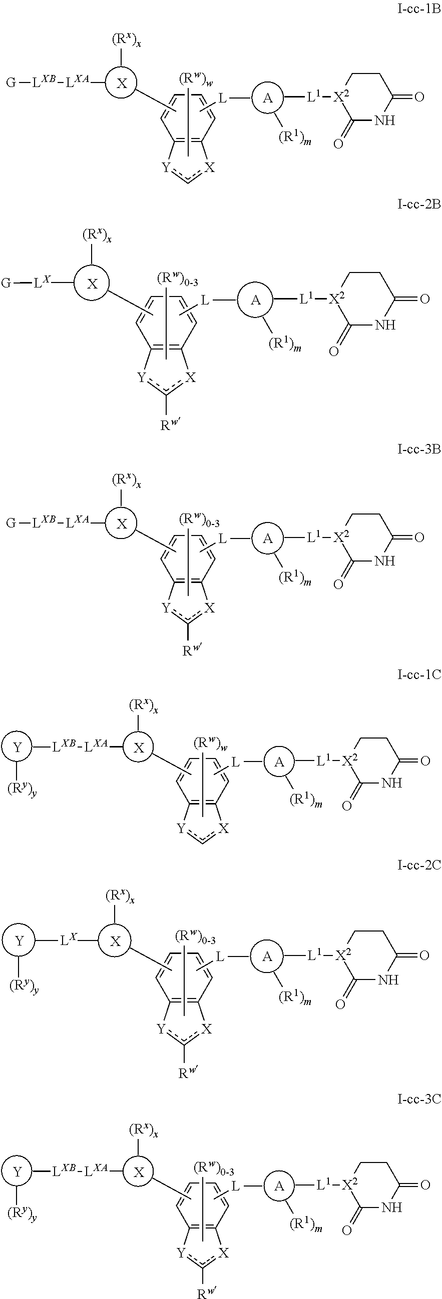

or a pharmaceutically acceptable salt thereof.
122. The compound of any one of embodiments 101-111 or 113-120 wherein the compound is of formula I-cc-1D, I-cc-2D, I-cc-3D, I-cc-1E, I-cc-2E, or I-cc-3E:

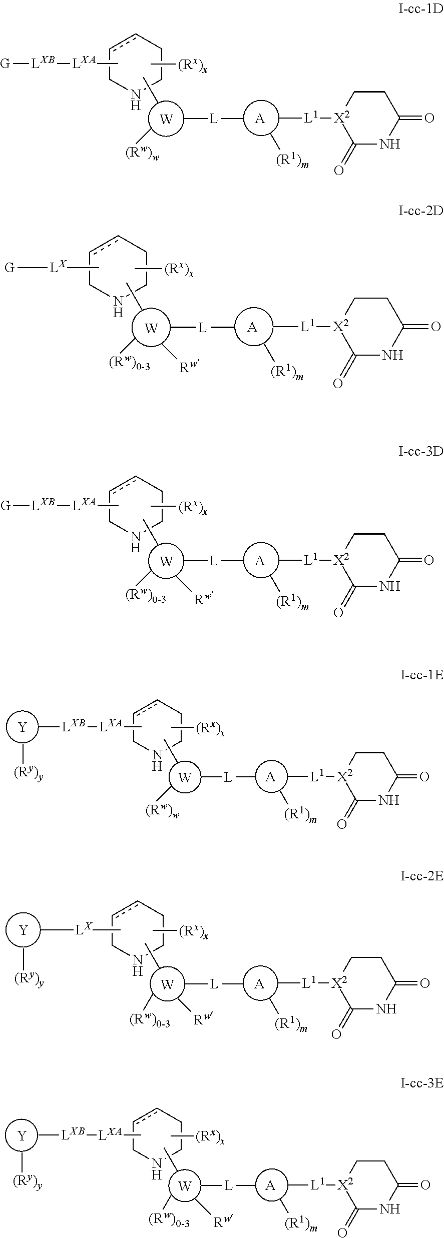

or a pharmaceutically acceptable salt thereof.
123. The compound of any one of embodiments 101-111 or 113-120 wherein the compound is of formula I-cc-1F, I-cc-2F, I-cc-3F, I-cc-1G, I-cc-2G. or I-cc-3G:

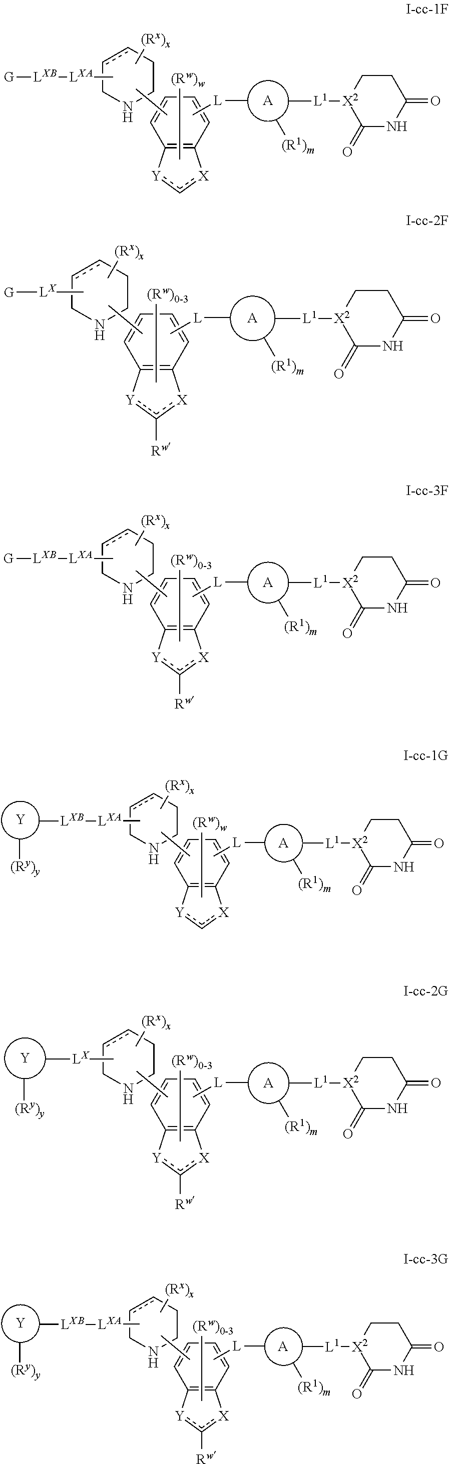

or a pharmaceutically acceptable salt thereof.
124. The compound of any one of embodiments 101-111 or 113-120 wherein the compound is of formula I-cc-1H, I-cc-2H, I-cc-3H, I-cc-J I-cc-2J, or I-cc-3J:

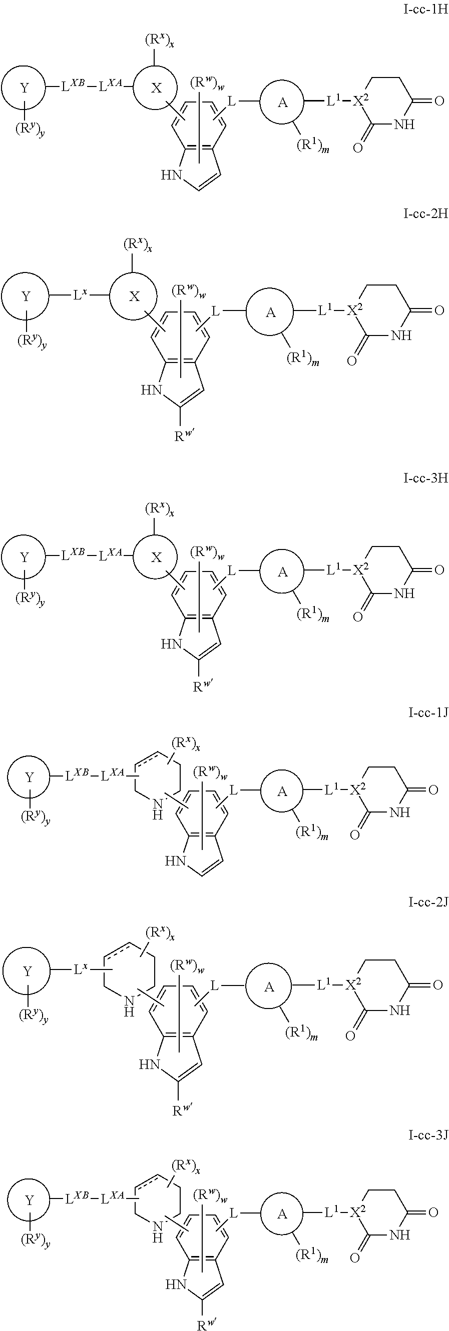

or a pharmaceutically acceptable salt thereof.
125. The compound of any one of embodiments 101-124, wherein. L is:

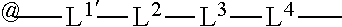

wherein:
@ a represents the point of attachment to Ring W;
L^{1′} is a covalent bond or an optionally substituted bivalent, saturated or partially unsaturated, straight or branched C₁₋₆ hydrocarbon chain, wherein 1-2 methylene units of L_{1′} are optionally and independently replaced by -Cy^{L1}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—;
L² is a covalent bond or an optionally substituted bivalent, saturated or partially unsaturated, straight or branched C₁—hydrocarbon chain, wherein 1-2 methylene units of L² are optionally and independently replaced by -Cy^{L2}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—;
L³ is a covalent bond or an optionally substituted bivalent, saturated or partially unsaturated, straight or branched C₁₋₆ hydrocarbon chain, wherein 1-2 methylene units of L³ are optionally and independently replaced by -Cy^{L3}-, —O—, —NR—, —S—, —OC(O)—, —C(O)O—, —C(O)—, —S(O)—, —S(O)₂₋₁—NRS(O)₂—, —S(O)₂NR—, —NRC(O)—, —C(O)NR—, —OC(O)NR—, or —NRC(O)O—;
L⁴ is a covalent bond or an optionally substituted bivalent, saturated or partially unsaturated, straight or branched C₁₋₆ hydrocarbon chain, wherein 1-2 methylene units of L⁴ are optionally and independently replaced by -Cy^{L4}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—; and
each of -Cy^{L1}-, -Cy^{L2}-, -Cy^{L3}- and -Cy^{L4}- is independently -Cy-.
126. The compound of embodiment 125, wherein L^{1′} is a covalent bond.
127. The compound of embodiment 125, wherein L^{1′} is an optionally substituted bivalent, saturated or partially unsaturated, straight or branched C₁₋₆ hydrocarbon chain, wherein 1-2 methylene units of L^{1′} are optionally and independently replaced by -Cy^{L1}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—,
128. The compound of embodiment 125, wherein L^{1′} is an optionally substituted bivalent, saturated or partially unsaturated, straight or branched C₁₋₆ hydrocarbon chain.
129. The compound of embodiment 125, wherein L^{1′} is an optionally substituted bivalent, saturated or partially unsaturated, straight or branched C₁₋₃ hydrocarbon chain.
130. The compound of embodiment 125, wherein L^{1′} is -Cy^{L1}-.
131. The compound of any one of embodiments 125, 126, or 130, wherein -Cy^{L1}-is an optionally substituted phenylenyl or 5-6 membered saturated or partially unsaturated heteroarylenyl having 1-4 heteroatoms independently selected from nitrogen, oxygen, and sulfur.
132. The compound of any one of embodiments 125, 126, or 130-131, wherein -Cy^{L1}-is an optionally substituted phenylenyl.
133. The compound of any one of embodiments 125, 126, or 130-131, wherein -Cy^{L1}-is an optionally substituted pyridinylenyl, pyridazinylenyl, pyrimidinylenyl, pyrazinylenyl, or triazinylenyl.
134. The compound of any one of embodiments 125-133, wherein L² is a covalent bond.
135. The compound of any one of embodiments 125-133, wherein L² is an optionally substituted bivalent, saturated or partially unsaturated, straight or branched C₁₋₆ hydrocarbon chain, wherein 1-2 methylene units of L² are optionally and independently replaced by -Cy^{L2}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—,
136. The compound of any one of embodiments 125-133, wherein L² is an optionally substituted bivalent, saturated or partially unsaturated, straight or branched C₁₋₆ hydrocarbon chain.
137. The compound of any one of embodiments 125-133, wherein L² is an optionally substituted bivalent, saturated or partially unsaturated, straight or branched C₃ hydrocarbon chain.
138. The compound of any one of embodiments 125-133, wherein L² is -Cy^{L2}-.
139. The compound of embodiment 138, wherein -Cy^{L2}-is an optionally substituted 5-6 membered saturated or partially unsaturated carbocyclylenyl or heterocyclylenyl having 1-2 heteroatoms independently selected from nitrogen, oxygen, and sulfur.
140. The compound of embodiment 138 or 139, wherein -Cy^{L2}-is an optionally substituted piperadinylenyl or piperazinylenyl.
141. The compound of any one of embodiments 125-140, wherein L³ is a covalent bond.
142. The compound of any one of embodiments 125-140, wherein L³ is an optionally substituted bivalent, saturated or partially unsaturated, straight or branched C₁₋₆ hydrocarbon chain, wherein 1-2 methylene units of L³ are optionally and independently replaced by -Cy^{L3}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—,
143. The compound of any one of embodiments 125-140, wherein L³ is an optionally substituted bivalent, saturated or partially unsaturated, straight or branched C₁₋₆ hydrocarbon chain.
144. The compound of any one of embodiments 125-140, wherein L³ is an optionally substituted bivalent, saturated or partially unsaturated, straight or branched C₁₋₃ hydrocarbon chain.
145. The compound of any one of embodiments 125-140, wherein L³ is -Cy^{L3}-.
146. The compound of embodiment 145, wherein -Cy^{L3}-is an optionally substituted 5-6 membered saturated or partially unsaturated carbocyclylenyl or heterocyclylenyl having 1-2 heteroatoms independently selected from nitrogen, oxygen, and sulfur.
147. The compound of embodiment 145 or 146, wherein -Cy^{L3}-is an optionally substituted piperadinylenyl or piperazinylenyl.
148. The compound of any one of embodiment 125-147, wherein L⁴ is a covalent bond.
149. The compound of any one of embodiments 125-147, wherein L⁴ is an optionally substituted bivalent, saturated or partially unsaturated, straight or branched C₁₋₆ hydrocarbon chain, wherein 1-2 methylene units of L⁴ are optionally and independently replaced by -Cy^{L4}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—,
150. The compound of any one of embodiments 125-147, wherein L⁴ is an optionally substituted bivalent, saturated or partially unsaturated, straight or branched C₁₋₆ hydrocarbon chain.
151. The compound of any one of embodiments 125-147, wherein L⁴ is an optionally substituted bivalent, saturated or partially unsaturated, straight or branched C₁₋₃ hydrocarbon chain.
152. The compound of any one of embodiments 125-147, wherein L⁴ is -Cy^{L4}-.
153. The compound of any one of embodiments 125-147, wherein L is ^{@}-L^{1′}-L²-L³-.
154. The compound of any one of embodiments 125-141, wherein Lis ^{@}-L^{1′}-L²-.
155. The compound of embodiment 125, wherein L is:

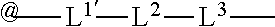

wherein:
L^{1′} is -Cy^{L1}-;
L² is -Cy^{L2}-; and
L³ is an optionally substituted bivalent, saturated or partially unsaturated, straight or branched C₁₋₆ hydrocarbon chain.
156. The compound of embodiment 125, wherein L is:

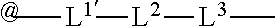

wherein:
L^{1′} is -Cy^{L1}-;
L² is -Cy^{L2}-; and
L³ is -Cy^{L3}.
157. The compound of embodiment 125, wherein L is:

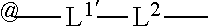

wherein:
@ represents the point of attachment to Ring W:
L^{1′} is -Cy^{L1}-; and
L² is -Cy^{L2}-.
158. The compound of embodiment 125, wherein the compound is of formula I-dd, I-dd-1, I-dd-2, I-dd-3, I-dd-A, I-dd-2A, I-dd-2A, or I-dd-3A:

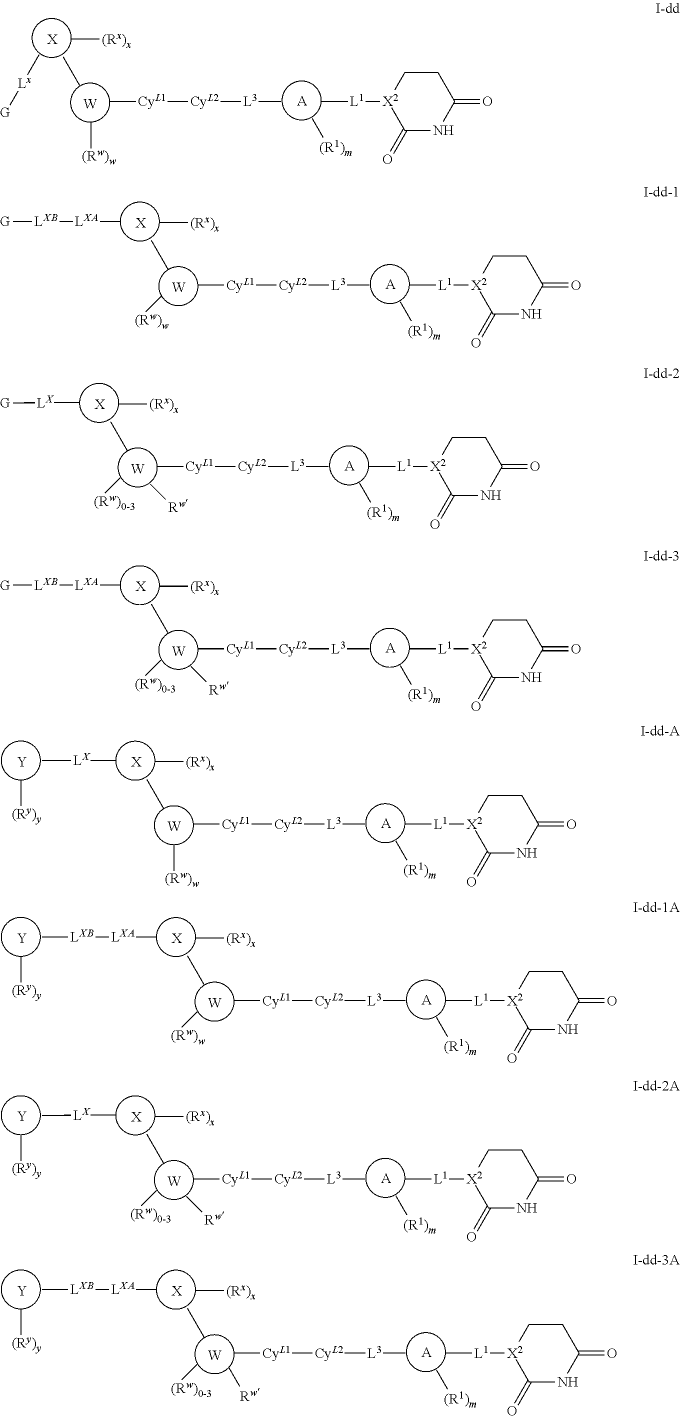

or a pharmaceutically acceptable salt thereof.
159. The compound of embodiment 125, wherein the compound is of formula I-dd-B, I-dd-1B, I-dd-2B, I-dd-3B, I-dd-C, I-dd-2C, I-dd-2C, or I-dd-3C:

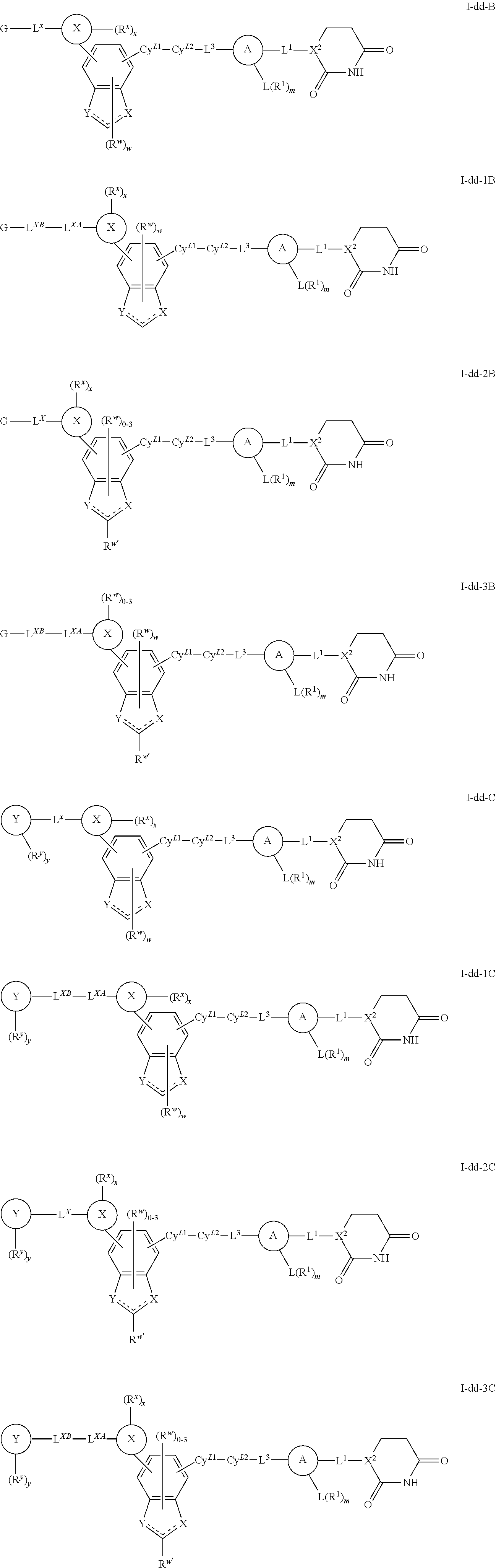

or a pharmaceutically acceptable salt thereof.
160. The compound of embodiment 125, wherein the compound is of formula I-dd-D, I-dd-1D, I-dd-2D, I-dd-3D, I-dd-E, I-dd-2E, I-dd-2E, or I-dd-3E:

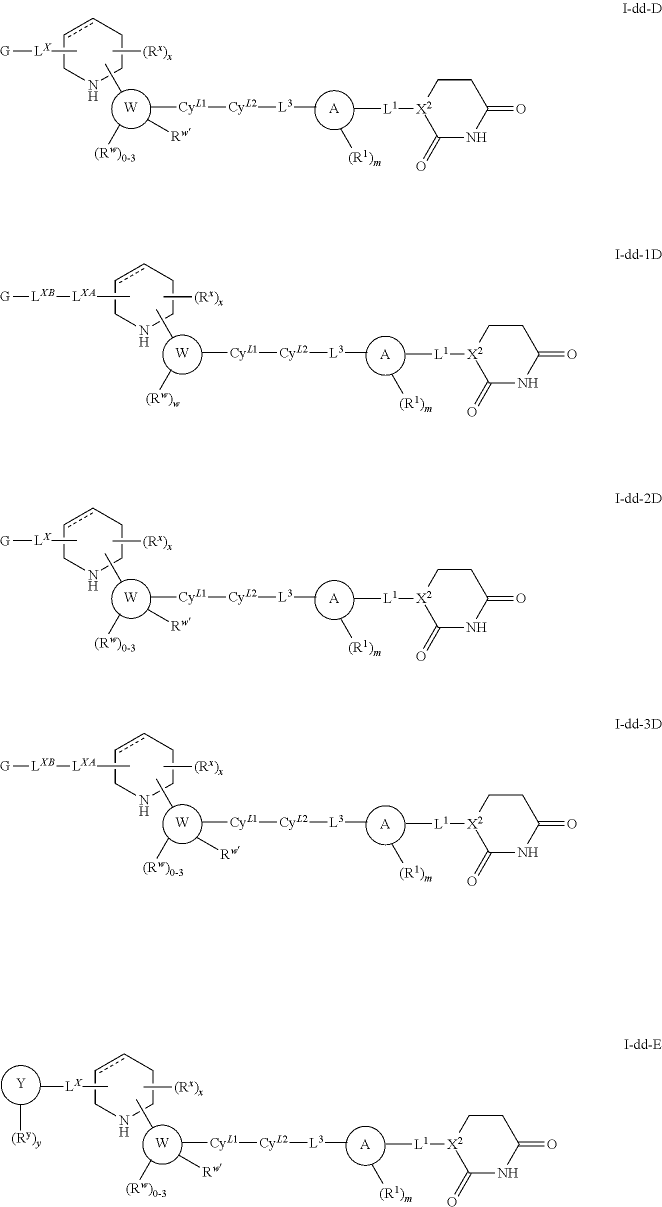

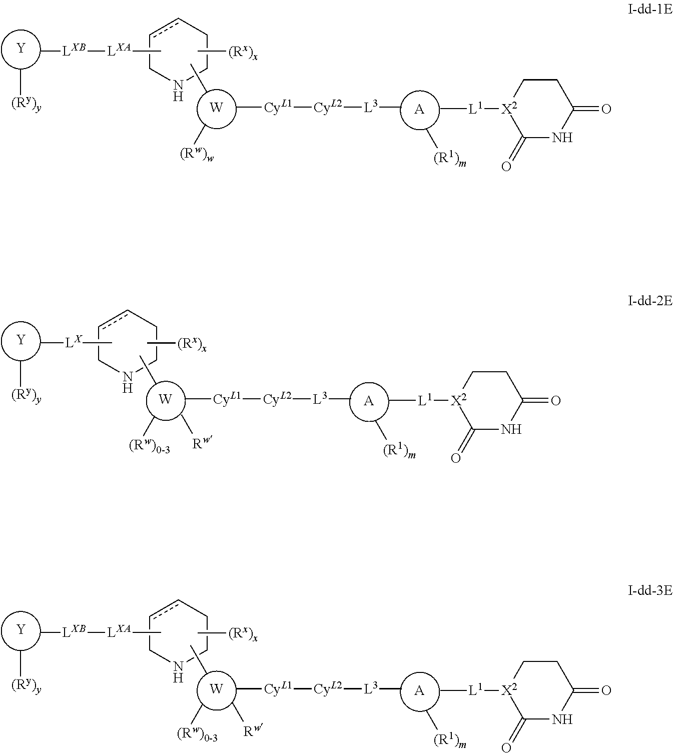

or a pharmaceutically acceptable salt thereof.
161. The compound of embodiment 125, wherein the compound is of formula I-dd-F, I-dd-1F, I-dd-2F, I-dd-3F, I-dd-G, I-dd-2G, I-dd-2G, or I-dd-3G:

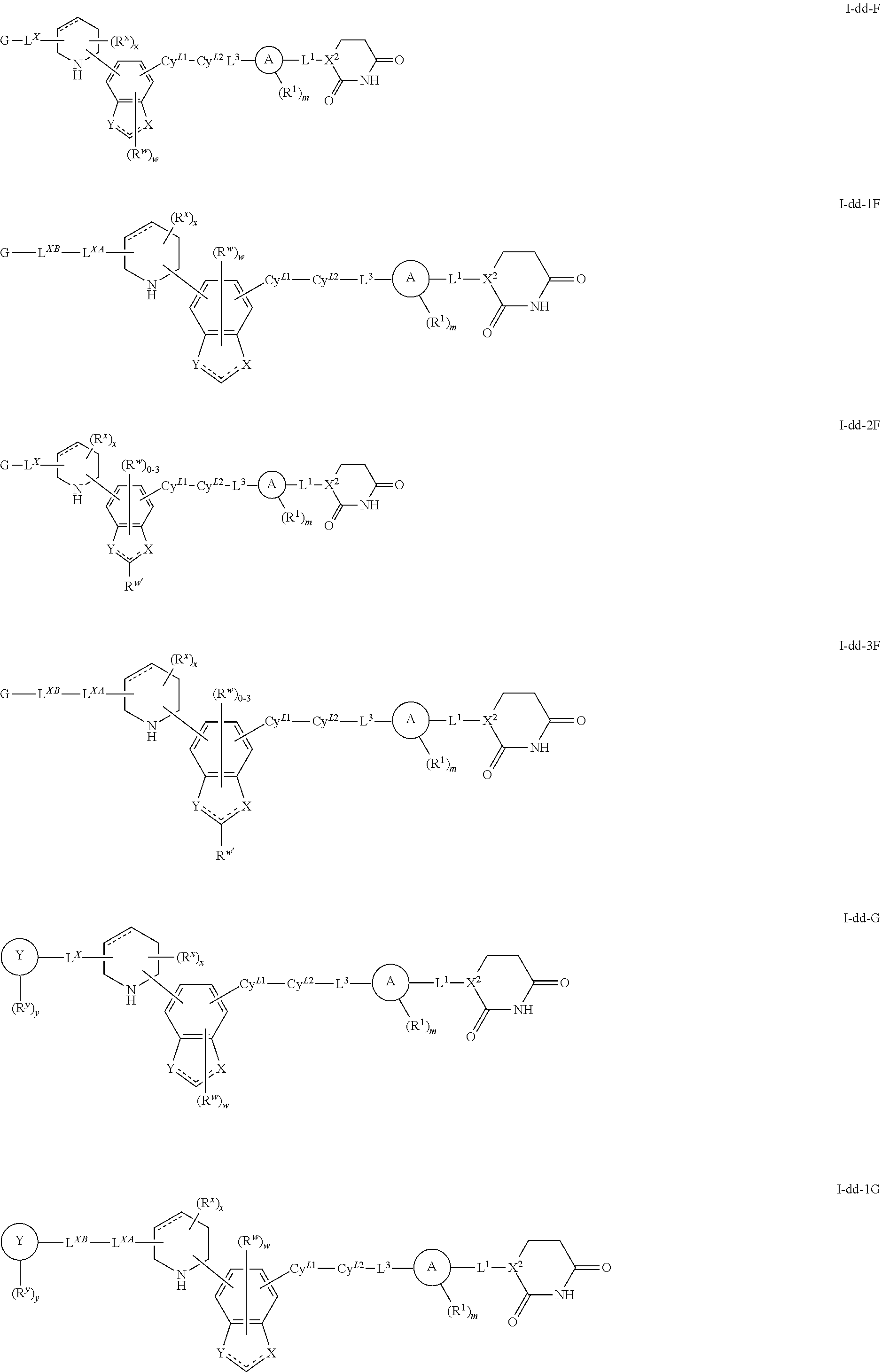

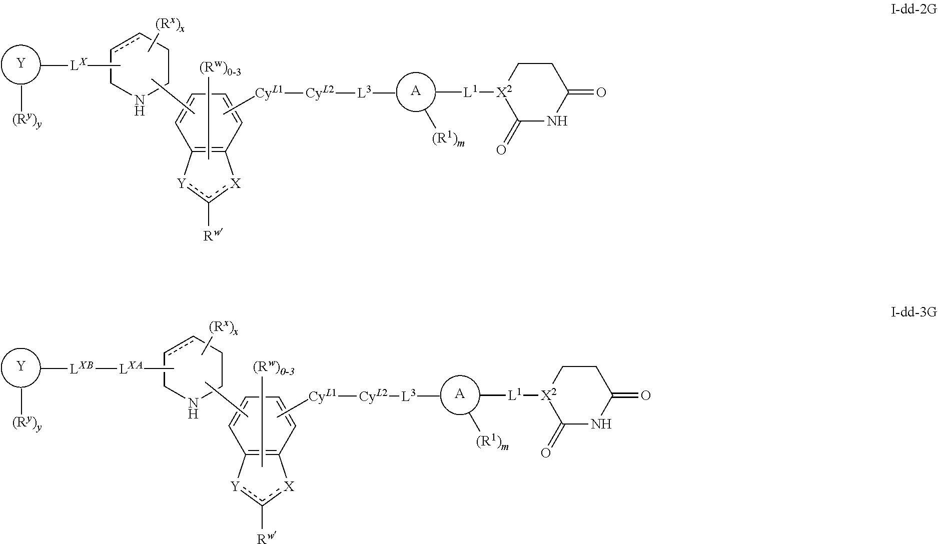

or a pharmaceutically acceptable salt thereof.
162. The compound of embodiment 125, wherein the compound is of formula I-dd-H, I-dd-1H, I-dd-2H, I-dd-3H, I-dd-J, I-dd-2J, I-dd-2J, or I-dd-3J:

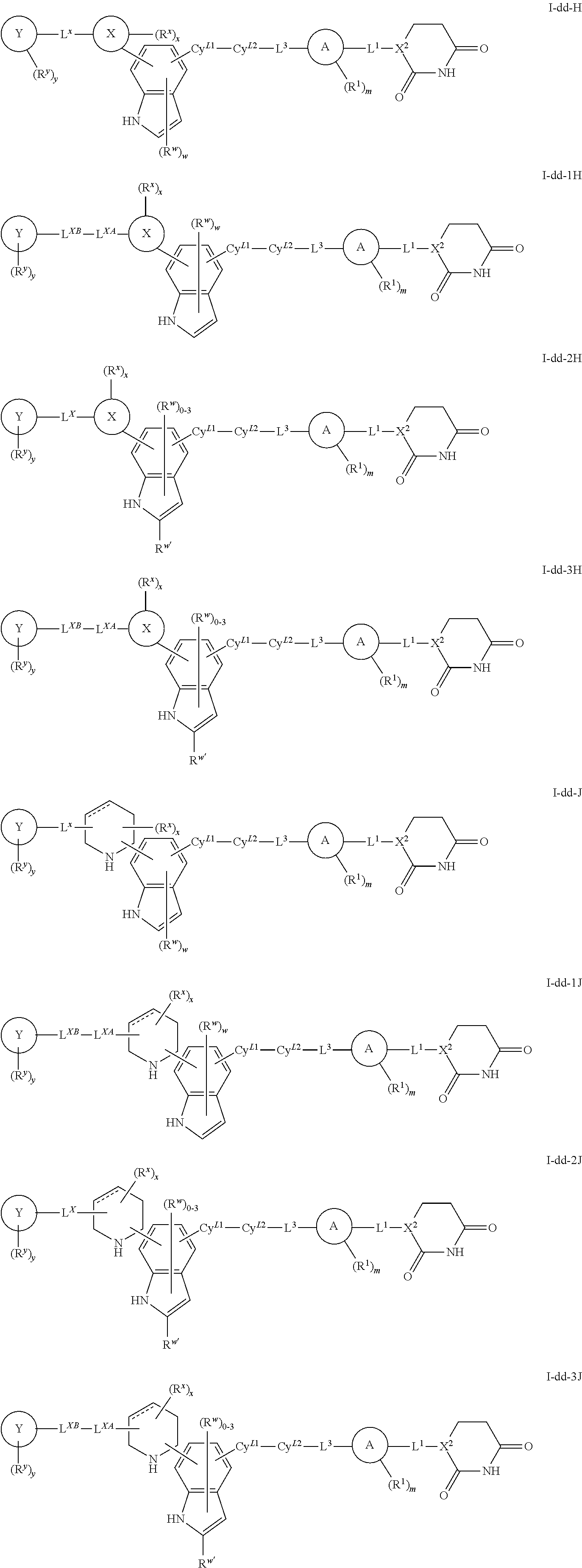

or a pharmaceutically acceptable salt thereof.
163. The compound of any one of embodiments 155-162, wherein -Cy^{L1}-is an optionally substituted phenylenyl or 5-6 membered saturated or partially unsaturated heteroarylenyl having 1-4 heteroatoms independently- selected from nitrogen, oxygen, and sulfur.
164. The compound of any one of embodiments 155-163, wherein -Cy^{L1}-is an optionally substituted phenylenyl.
165. The compound of any one of embodiments 155-163, wherein -Cy^{L1}-is an optionally substituted pyridinylenyl, pyridazinylenyl, pyrimidinylenyl, pyrazinylenyl, or triazinylenyl.
166. The compound of any one of embodiments 155-165, wherein -Cy^{L2}-is an optionally substituted 5-6 membered saturated or partially unsaturated carbocyclylenyl or heterocyclylenyl having 1-2 heteroatoms independently selected from nitrogen, oxygen, and sulfur.
167. The compound of any one of embodiments 155-166, wherein -Cy^{L2}-is an optionally substituted piperadinylenyl or piperazinylenyl.
168. The compound of any one of embodiments 155-167, wherein L³ is a covalent bond.
169. The compound of any one of embodiments 155-167, wherein L³ is an optionally substituted bivalent, saturated or partially unsaturated, straight or branched C_{1-_}hydrocarbon chain, wherein 1-2 methylene units of L³ are optionally and independently replaced by -Cy^{L3}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—,
170. The compound of any one of embodiments 155-167, wherein L³ is an optionally substituted bivalent, saturated or partially unsaturated, straight or branched C₁₋₆ hydrocarbon chain.
171. The compound of any one of embodiments 155-167, wherein L³ is an optionally substituted bivalent, saturated or partially unsaturated, straight or branched C₁₋₃ hydrocarbon chain.
172. The compound of any one of embodiments 155-167, wherein L³ is -Cy^{L3}-.
173. The compound of embodiment 172, wherein -Cy^{L3}-is an optionally substituted 5-6 membered saturated or partially unsaturated carbocyclylenyl or heterocyclylenyl having 1-2 heteroatoms independently selected from nitrogen, oxygen, and sulfur.
174. The compound of embodiment 172 or 173, wherein -Cy^{L3}-is an optionally substituted piperadinylenyl or piperazinylenyl.
175. The compound of any one of embodiments 101-174, wherein:
Ring A is a ring selected from phenylenyl, naphthalenyl, pyridinylenyl,

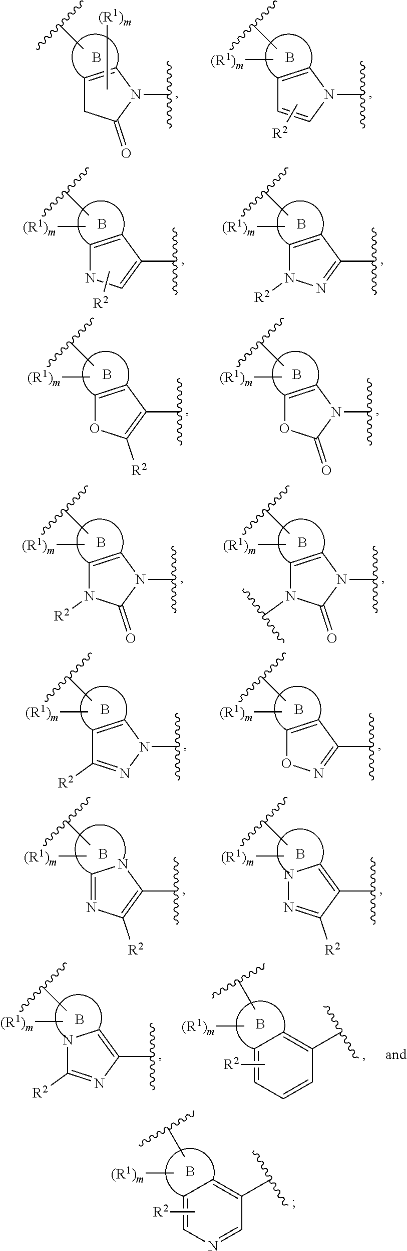

Ring B is a fused ring selected from benzo, a saturated or partially unsaturated 4-7 membered carbocyclyl, a saturated or partially unsaturated 4-7 membered heterocyclyl having 1-2 heteroatoms independently selected from nitrogen, oxygen, and sulfur, and a 5-6 membered heteroaryl containing 1-4 heteroatoms independently selected from nitrogen, oxygen, and sulfur:
each R¹ is independently R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)N(R)OR, —CR₂N(R)C(O)R, —CR₂N(R)C(O)NR₂, —CFR₂, —CF₂R, —CF₃, —CR₂(OR), —CR₂(NR₂), —OC(O)R, —OC(O)NR₂, —OP(O)R₂, —OP(O)(OR)₂, —OP(O)(OR)NR₂, —OP(O)(NR₂)₂, —N(R)C(O)OR—N(R)C(O)R, —N(R)C(O)NR₂, —N(R)S(O)₂R, —N(R)P(O)R₂, —N(R)P(O)(OR)₂, —N(R)P(O)(OR)NR₂, —N(R)P(O)(NR₂)₂, or —N(R)S(O)₂R: or:
   two R¹ groups of Ring A are taken together with their intervening atoms to form an optionally substituted ring selected from a 3-10 membered saturated or partially unsaturated carbocyclyl or heterocyclyl having 1-4 heteroatoms independently selected from nitrogen, oxygen, or sulfur; benzo; or a 3-10 membered heteroaryl having 1-4 heteroatoms independently selected from nitrogen, oxygen, or sulfur;
R² is hydrogen, halogen, C₁₋₆ alkyl, C₃₋₆ cycloalkyl, C₁₋₆ haloalkyl, —OC₁₋₆ alkyl, —OC₃₋₆ cycloalkyl, or —OC₁₋₆ haloalkyl; or:
   an R² and an R¹ are taken together with their intervening atoms to form an optionally substituted ring selected from a 3-10 membered saturated or partially unsaturated carbocyclyl or heterocyclyl having 1-4 heteroatoms independently selected from nitrogen, oxygen, or sulfur; benzo; or a 5-10 membered heteroaryl having 1-4 heteroatoms independently selected from nitrogen, oxygen, or sulfur.
176. The compound of any one of embodiments 101-175, wherein the structure

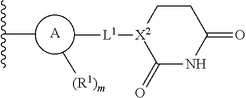

is of any one of formula I-aa-10′, I-aa-11′, I-aa-12′, I-aa-13′, or I-aa-14′:

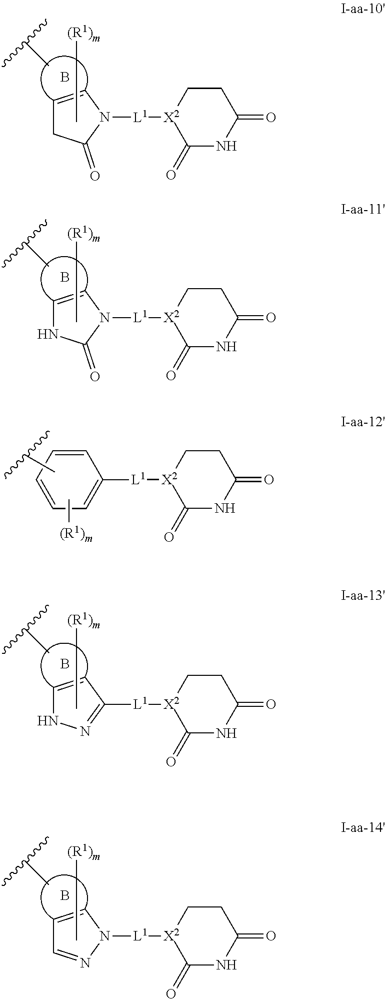

or a pharmaceutically acceptable salt thereof.
177. The compound of any one of embodiments 101-176, wherein the structure

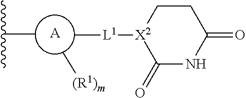

is of any one of formula I-aa-10a′, I-aa-11a′, I-aa-12a′, I-aa-12b′, I-aa-13a′, or I-aa-14a′:

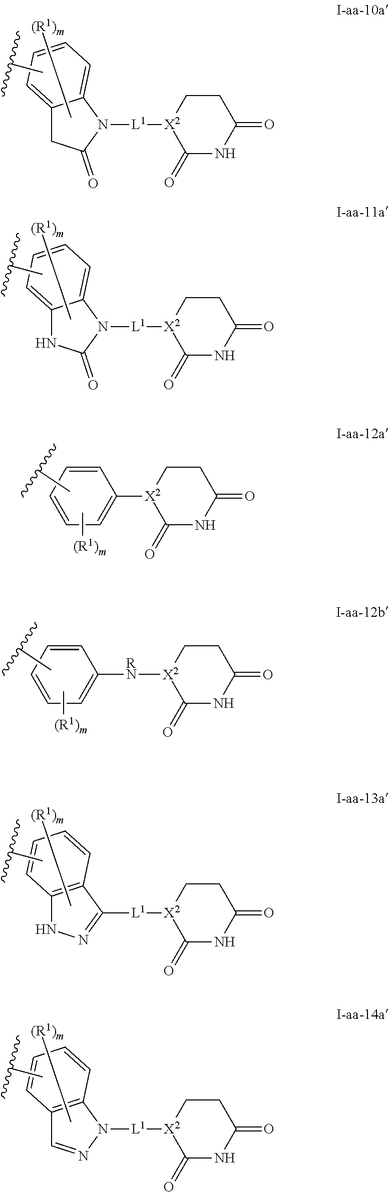

or a pharmaceutically acceptable salt thereof.
178. The compound of any one of embodiments 101-177, wherein the structure

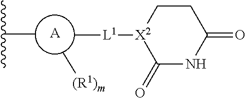

is of any one of formula I-aa-2′, I-aa-3′, or I-aa-4′:

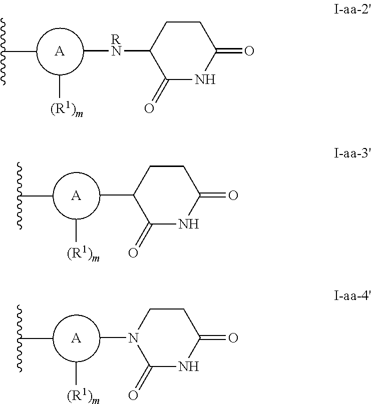

or a pharmaceutically acceptable salt thereof.
179. A pharmaceutical composition comprising a compound of any one of embodiments 1-178, and a pharmaceutically acceptable carrier, adjuvant, or vehicle.
180. A method of degrading STAT6 in a patient or biological sample comprising administering to said patient, or contacting said biological sample with a compound of any one of embodiments 1-178, or a pharmaceutical composition thereof.
181. A method of treating an STAT6-mediated disorder, disease, or condition in a patient comprising administering to said patient a compound of any one of embodiments 1-178, or a pharmaceutical composition thereof.
182. The method of embodiment 181, wherein STAT6-mediated disorder, disease, or condition is cancer, a neurodegenerative disorder, a viral disease, an autoimmune disease, an inflammatory disorder, a hereditary disorder, a hormone-related disease, a metabolic disorder, conditions associated with organ transplantation, immunodeficiency disorders, a destructive or overgrowing bone disorder, a proliferative disorder, an infectious disease, a condition associated with cell death, thrombin-induced platelet aggregation, liver disease, pathologic immune conditions involving T cell activation, a cardiovascular disorder, or a CNS disorder.

LENGTHY TABLES
The patent application contains a lengthy table section. A copy of the table is available in electronic form from the USPTO web site (https://seqdata.uspto.gov/docdetail?docId=US20260124309A1). An electronic copy of the table will also be available from the USPTO upon request and payment of the fee set forth in 37 CFR 1.19(b)(3).

1. A compound of formula I-bb: 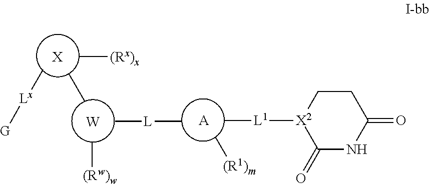
or a pharmaceutically acceptable salt thereof, wherein
Ring W is a 9-membered bicyclic heteroaryl with 1-4 heteroatoms independently selected from nitrogen, oxygen, and sulfur;
Ring X is a 6-membered saturated or partially unsaturated carbocyclylenyl or heterocyclylenyl with 1-3 heteroatoms independently selected from nitrogen, oxygen, and sulfur;
G is hydrogen, halogen, or 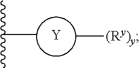
Ring Y is a 3- to 6-membered saturated or partially unsaturated carbocyclyl, or 4- to 6-membered monocyclic saturated or partially unsaturated heterocyclyl or heteroaryl ring with 1-4 heteroatoms independently selected from nitrogen, oxygen, or sulfur;
R^{w}, R^{x}, and R^{y} are independently selected from hydrogen, R^{A}, halogen, —CN, —NO₂, —OR, —SR, —NR₂, —SiR₃, —S(O)R, —S(O)₂R, —S(O)(NR)R, —S(O)₂NR₂, —S(O)R, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R;
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-10 membered monocyclic or bicyclic heteroaryl ring having 1-4 heteroatoms independently selected from nitrogen, oxygen, and sulfur;
L^{X} is a covalent bond or a C₁s bivalent straight or branched saturated or unsaturated hydrocarbon chain wherein 1-2 methylene units of the chain are independently and optionally replaced with —O—, —C(O)—, —C(S)—, —CR₂—, —NR—, —S—, —S(O)—, or —S(O)₂—;
each of w, x, and y are independently 0, 1, 2, 3, or 4;
L is a covalent bond, -Cy-(CH₂)₁₋₁₀—, —(CH₂)₁₋₁₀-Cy-(CH₂)₁₋₁₀—, —(CH₂)₁₋₁₀-Cy-(CH₂CH₂O)₁₋₁₀CH₂CH₂—, -Cy-(CH₂)₁₋₁₀-Cy-, -Cy-(CH₂)₁₋₁₀-Cy-(CH₂)₁₋₁₀—, -Cy-(CH₂)₁₋₁₀-Cy-(CH₂)₁₋₁₀-Cy-, —(CH₂)₁₋₁₀-Cy-(CH₂)₁₋₁₀-Cy-(CH₂)₁₋₁₀—, -Cy-Cy-, -Cy-Cy-(CH₂)₁₋₁₀—, -Cy-(CH₂)₁₋₁₀-Cy-(CH₂)₁₋₁₀—, -Cy-Cy-Cy-, -Cy-Cy-(CH₂)₁₋₁₀-Cy-, or -Cy-Cy-(CH₂)₁₋₁₀-Cy-(CH₂)₁₋₁₀—;
each -Cy- is independently an optionally substituted bivalent ring selected from phenylenyl, an 8-10 membered bicyclic arylenyl, a 4-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur:
X² is N or CH;
each R¹ is independently R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)N(R)OR, —C(R)₂N(R)C(O)R, —C(R)₂N(R)C(O)NR₂, —OC(O)R, —OC(O)NR₂, —OP(O)R₂, —OP(O)(OR)₂, —OP(O)(OR)(NR₂), —OP(O)(NR₂)₂, —N(R)C(O)OR, —N(R)C(O)R, —N(R)C(O)NR₂, —N(R)S(O)₂R, —NP(O)R₂, —N(R)P(O)(OR)₂, —N(R)P(O)(OR)(NR₂), —N(R)P(O)(NR₂)₂, —N(R)S(O)₂R;
L¹ is a covalent bond, —C(O)—, —NR—, —O—, —S—, —S(O)₂, —NRC(O)—, or —C(O)NR—; L¹ is a covalent bond, —C(O)—, —NR—, —O—, —S—, —S(O)₂, —NRC(O)—, or —C(O)NR—;
Ring A is phenylenyl or a 5 to 10-membered saturated or partially unsaturated monocyclic or bicyclic heterocyclylenyl or heteroarylenyl having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same or adjacent atoms or R^{B} and an R group are taken together with their intervening atoms to form an optionally substituted 3-11 membered saturated or partially unsaturated monocyclic, bicyclic, bridged bicyclic, or spirocyclic carbocyclic or heterocyclic ring having 0-3 heteroatoms independently selected from nitrogen, oxygen, and sulfur;
m is 0, 1, 2, 3, 4, or 5.

2. A compound of formula I-aa-1: 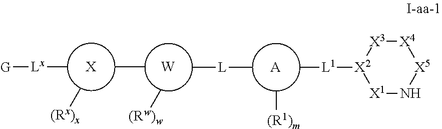
or a pharmaceutically acceptable salt thereof, wherein:
Ring W is a 9-membered bicyclic heteroaryl with 1-4 heteroatoms independently selected from nitrogen, oxygen, and sulfur;
Ring X is a 6-membered saturated or partially unsaturated carbocyclylenyl or heterocyclylenyl with 1-3 heteroatoms independently selected from nitrogen, oxygen, and sulfur;
G is hydrogen, halogen, or 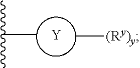
Ring Y is a 3- to 6-membered saturated or partially unsaturated carbocyclyl, or 4- to 6-membered monocyclic saturated or partially unsaturated heterocyclyl or heteroaryl ring with 1-4 heteroatoms independently selected from nitrogen, oxygen, or sulfur;
R^{x} and R^{y} are independently selected from hydrogen, R^{A}, halogen, —CN, —NO₂, —OR, —SR, —NR₂, —SiR₃, —S(O)R, —S(O)₂R, —S(O)(NR)R, —S(O)₂NR₂, —S(O)R, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R;
each R^{w} is independently selected from hydrogen, R^{A}, R^{B′} halogen, —CN, —NO₂, —OR, —SR, —NR₂, —SiR₃, —S(O)R, —S(O)₂R, —S(O)(NR)R, —S(O)₂NR₂, —S(O)R, —C(O)R, —C(S)R, —C(NR)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, and —NRS(O)₂R;
each R^{A} is independently an optionally substituted group selected from C₁₋₆ aliphatic, phenyl, naphthalenyl, a 3-7 membered saturated or partially unsaturated carbocyclic or heterocyclic ring having 1-2 heteroatoms independently selected from nitrogen, oxygen, and sulfur, a 5-10 membered monocyclic or bicyclic aryl or heteroaryl ring having 1-4 heteroatoms independently selected from nitrogen, oxygen, and sulfur or a 7-11 membered bicyclic heterocyclic ring having 1-4 heteroatoms independently selected from nitrogen, oxygen, and sulfur;
each R^{B′} is independently -L^{B}-Cy^{B1}-H or -L^{B}-Cy^{B1}-Cy^{B2};
each L^{B} is independently a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(NR)R—, —CR₂—, —CF₂—, —CRF—, —CR(OR)—, —NR—, —S—, —S(O)—, —S(O)₂— —S(O)(NR)— or —CR═CR—;
each Cy^{B1} is independently an optionally substituted ring selected from phenylenyl, a 3-10 membered saturated or partially unsaturated monocyclic or bicyclic carbocyclylenyl or heterocyclylenyl having 1-4 heteroatoms independently selected from nitrogen, oxygen, and sulfur, and a 5-10 membered monocyclic or bicyclic arylenyl or heteroarylenyl having 1-4 heteroatoms independently selected from nitrogen, oxygen, and sulfur;
each Cy^{B2} is independently an optionally ring selected from phenyl, a 3-10 membered saturated or partially unsaturated monocyclic or bicyclic carbocyclic or heterocyclic ring having 1-2 heteroatoms independently selected from nitrogen, oxygen, and sulfur, and a 5-10 membered monocyclic or bicyclic aryl or heteroaryl with 1-4 heteroatoms independently selected from nitrogen, oxygen and sulfur;
each R is independently hydrogen, or an optionally substituted group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-10 membered heteroaryl ring having 1-4 heteroatoms independently selected from nitrogen, oxygen, and sulfur, or:
two R groups on the same atom or adjacent atoms are optionally taken together with their intervening atoms to form a 3-10 membered saturated or partially unsaturated monocyclic, bicyclic, bridged bicyclic, or spirocyclic ring having 0-3 heteroatoms, in addition to the atom or adjacent atoms to which they are attached, independently selected from nitrogen, oxygen, and sulfur;
L^{x} is a covalent bond or an optionally substituted C₁₋₅ bivalent straight or branched saturated or unsaturated hydrocarbon chain wherein 1-2 methylene units of the chain are independently and optionally replaced with —O—, —C(O)—, —C(S)—, —CR₂—, —NR—, —C(NR)—, —S—, —S(O)—, —S(O)₂—, —S(O)(NR)—, or —CR═CR—;
each of w, x, and y are independently 0, 1, 2, 3, or 4:
L is a covalent bond or an optionally substituted bivalent, saturated or partially unsaturated, straight or branched C₁₋₂₀ hydrocarbon chain, wherein 0-4 methylene units of L are independently replaced by -Cy-, —CHF—, —CF₂—, —O—, —NR—, —SiR₂—, —Si(OH)R—, —Si(OH)₂—, —P(O)OR—, —P(O)R—, —P(O)NR₂—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—, 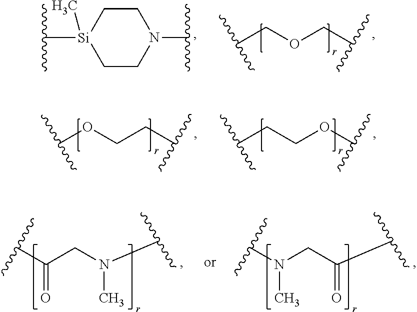
wherein:
each -Cy- is independently an optionally substituted bivalent ring selected from phenylenyl, an 8-10 membered bicyclic arylenyl, a 3-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-3 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur;
each r is independently 0, 1, 2, 3, 4, 5, 6, 7, 8, 9, or 10; and
X¹ and X⁵ are independently a covalent bond, —CR₂—, —SO₂—, —S(O)—, —P(O)R—, —P(O)OR—, —P(O)N(R)₂—, —C(O)—, —C(S)—, or 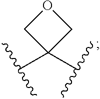 [SMILES: *C1(*)COC1]
X² is N, C—R^{B}, Si—R^{B}, or P═O;
X³ and X⁴ are independently a covalent bond, —CR₂—, —CF₂—, —O—, —S—, or
X³—X⁴ is —CR═CR—;
each R¹ is independently —H, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)N(R)OR, —C(R)₂N(R)C(O)R—C(R)₂N(R)C(O)NR₂, —OC(O)R, —OC(O)NR₂, —OP(O)R₂, —OP(O)(OR)₂, —OP(O)(OR)(NR₂), —OP(O)(NR₂)₂, —N(R)C(O)OR, —N(R)C(O)R, —N(R)C(O)NR₂, —N(R)S(O)₂R, —NP(O)R₂, —N(R)P(O)(OR)₂, —N(R)P(O)(OR)(NR₂), —N(R)P(O)(NR₂)₂, —N(R)S(O)₂R; or:
two R¹ groups of Ring A are taken together with their intervening atoms to form an optionally substituted ring selected from a 3-10 membered saturated or partially unsaturated carbocyclyl or heterocyclyl having 1-4 heteroatoms independently selected from nitrogen, oxygen, or sulfur; benzo: or a 5-10 membered heteroaryl having 1-4 heteroatoms independently selected from nitrogen, oxygen, or sulfur:
m is 0, 1, 2, 3, 4, or 5;
each R^{B} is independently, hydrogen, halogen, —CN, —OR, —SR, —S(O)R, —S(O)₂R, —NR₂, —P(O)(OR)₂, —P(O)(NR₂)OR, —P(O)(NR₂)₂, —Si(OH)₂R, —Si(OH)R₂, —SiR₃, or an optionally substituted Cia aliphatic;
L¹ is a covalent bond or a C₁₋₃ bivalent hydrocarbon chain wherein 1-2 methylene units of the chain are independently and optionally replaced with —C(O)—, —C(S)—, —CR₂—, —CF₂—, —NR—, —O—, —S—, or —S(O)₂; and
Ring A is phenylenyl, naphthalenyl, a 4-7 membered saturated or partially unsaturated carbocyclylenyl, a 4-7 membered saturated or partially unsaturated heterocyclylenyl having 1-3 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, an 8-10 membered bicyclic heteroarylenyl having 1-5 heteroatoms independently selected from nitrogen, oxygen, and sulfur, an 8-15 membered saturated or partially unsaturated tricyclic heterocyclylenyl having 1-5 heteroatoms independently selected from nitrogen, oxygen, and sulfur, or an 8-15 membered tricyclic heteroarylenyl having 1-5 heteroatoms independently selected from nitrogen, oxygen, and sulfur.

3. The compound of claim 1 or 2, wherein Ring W is: 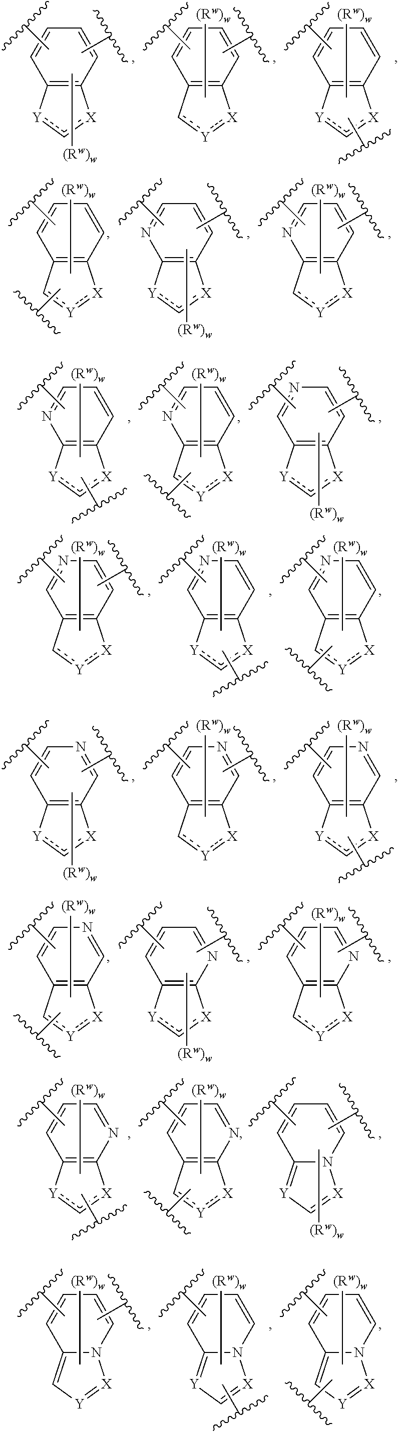 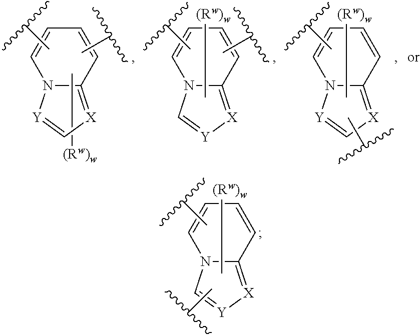
wherein:
each of X and Y is independently N, NH, N—R^{W}, —O—, —S—, C—H, C—R^{W}, CH₂, CH(R^{W}), or C(R^{W})₂, as allowed by valency.

4. The compound of claim 3, wherein:
X is C—R^{w} or CH, and Y is N—R^{w};
X is C—R^{w} or CH, and Y is S;
X is C—R^{w} or CH, and Y is O;
X is N—R^{w} or NH, and Y is C—R^{w} or CH;
X is S, and Y is C—R^{w} or CH; or
X is O, and Y is C—R^{w} or CH.

5. The compound of any one of claims 1-4, wherein Ring X is

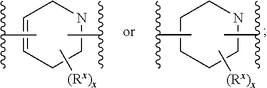

6. The compound of any one of claims 1-5, wherein Ring Y is a 5 membered monocyclic heteroaryl ring with 1-4 heteroatoms independently selected form nitrogen, oxygen, and sulfur.

7. The compound of any one of claims 1-6, wherein Ring Y is pyrazolyl, imidazolyl, or triazolyl.

8. The compound of any one of claims 1-7, wherein Lx is a covalent bond or ^{#}-L^{XA}-L^{XB}-, wherein:
^{#} represents the point of attachment to Ring X:
L^{XA} is —C(O)—, —C(S)—, —CR₂—, —S(O)—, or —S(O)₂—; and
L^{XD} is a covalent bond or an optionally substituted C₁₋₄ bivalent straight or branched saturated or unsaturated hydrocarbon chain wherein 1-2 methylene units of the chain are independently and optionally replaced with —O—, —C(O)—, —C(S)—, —CR₂—, —NR—, —S—, —S(O)—, or —S(O)₂—.

9. The compound of claim 8, wherein L^{XA} is —C(O)—.

10. The compound of claim 8 or 9, wherein L^{XB} is an optionally substituted C₁₋₄ bivalent straight or branched saturated or unsaturated hydrocarbon chain.

11. The compound of claims 1-10, wherein an occurrence of R^{w} is —C(S)R, —C(NR)R, —S(O)R, —S(O)₂R, —S(O)(NR)R—S(O)₂NR₂, —S(O)R, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, or —NRS(O)₂R.

12. The compound of claims 1-11, wherein an occurrence of R^{w} is an optionally substituted 5-6 membered monocyclic heteroaryl ring having 1-4 heteroatoms independently selected from nitrogen, oxygen, and sulfur.

13. The compound of any one of claims 1-12, wherein an occurrence of R^{w} is halogen (e.g., fluoro).

14. The compound of any one of claims 1-13, wherein y is 0.

15. The compound of any one of claims 1-14, wherein:
the compound is of formula I-bb-I, I-bb-IX, I-bb-A, I-bb-B, I-bb-C, I-bb-D, I-bb-E, or I-bb-F: 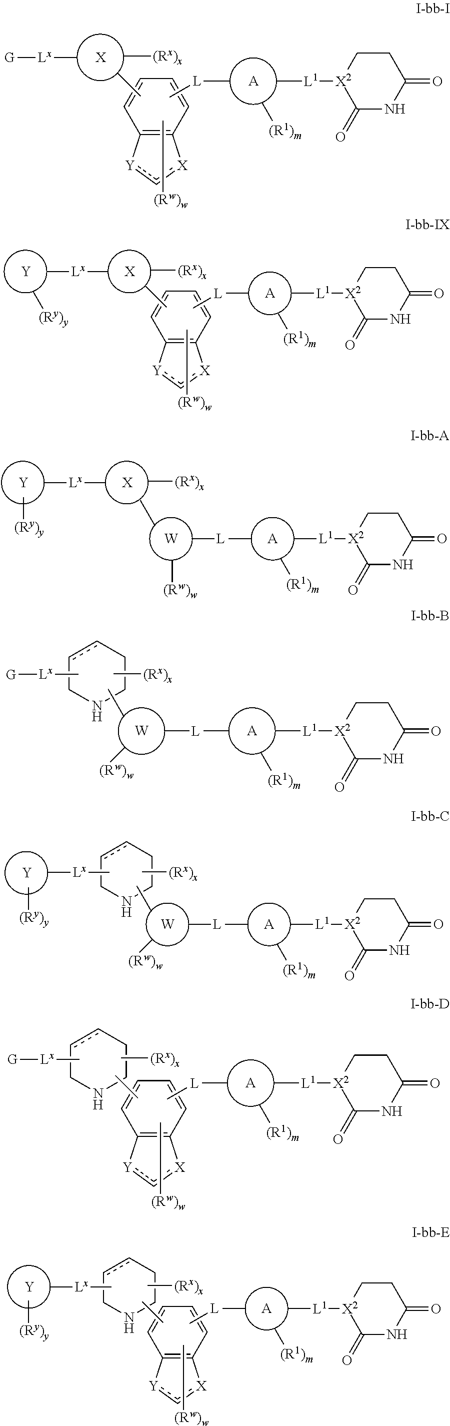 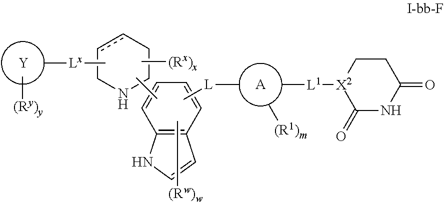
or a pharmaceutically acceptable salt thereof; or
the compound is of formula I-cc-1 I-cc-2, or I-cc-3: 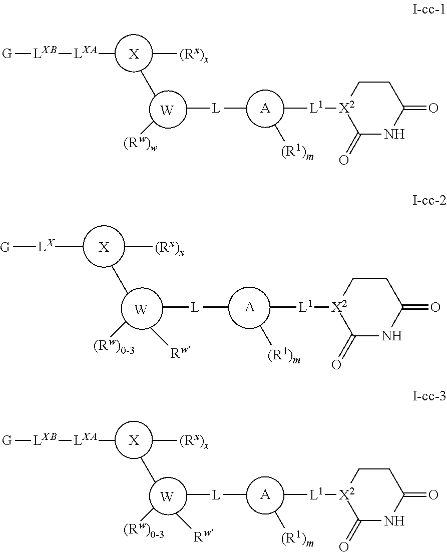
or a pharmaceutically acceptable salt thereof, wherein:
R_{w′} is —S(O)R, —S(O)₂R, —S(O)(NR)R, —S(O)₂NR₂, —C(O)R, —C(S)R, —C(NR)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)R, —NRC(O)N(R)₂, —NRS(O)₂R, or an optionally substituted group selected from C₁₋₆ aliphatic, phenyl, naphthalenyl, a 3-7 membered saturated or partially unsaturated carbocyclic or heterocyclic ring having 1-2 heteroatoms independently selected from nitrogen, oxygen, and sulfur, and a 5-10 membered monocyclic or bicyclic heteroaryl ring having 1-4 heteroatoms independently selected from nitrogen, oxygen, and sulfur.

16. The compound of claim 15, wherein:
R^{w′} is an optionally substituted 5-6 membered heteroaryl having 1-4 heteroatoms independently selected from nitrogen, oxygen, and sulfur; or
R^{w′} is 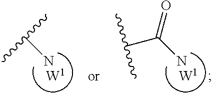
and
Ring W¹ is an optionally substituted 3-7 membered saturated or partially unsaturated heterocyclic ring having 1 additional heteroatom selected from nitrogen, oxygen, and sulfur, or a 5-6 membered monocyclic heteroaryl ring having 1-3 additional heteroatoms independently selected from nitrogen, oxygen, and sulfur; or
R^{w′} is 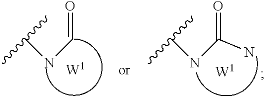
and
Ring W¹ is an optionally substituted 3-7 membered saturated or partially unsaturated heterocyclic ring having 1 additional heteroatom selected from nitrogen, oxygen, and sulfur, or a 5-6 membered monocyclic heteroaryl ring having 1-3 additional heteroatoms independently selected from nitrogen, oxygen, and sulfur; or
R^{w′} is 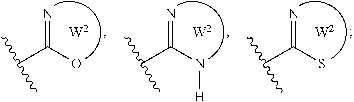
and
Ring W² is an optionally substituted 3-7 membered partially unsaturated heterocyclic ring, or a 5-6 membered monocyclic heteroaryl ring having 1-2 additional heteroatoms independently selected from nitrogen, oxygen, and sulfur.

17. The compound of claim 15, wherein R^{w′} is —S(O)R, —S(O)₂R, —S(O)(NR)R—S(O)₂NR₂—S(O)R, —C(S)R, —C(NR)R, —C(O)R, —C(O)OR, —C(O)NR₂, or —C(O)NROR.

18. The compound of any one of claims 1-17, wherein:
the compound is of formula I-cc-1A, I-cc-2A, or I-cc-3A: 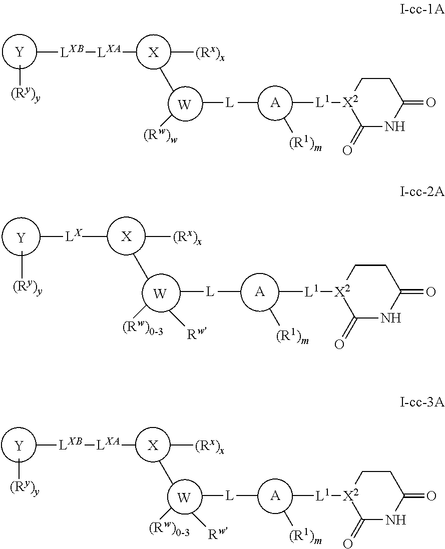
or a pharmaceutically acceptable salt thereof; or
the compound is of formula I-cc-1B, I-cc-2B, I-cc-3B, I-cc-1C, I-cc-2C, or I-cc-3C: 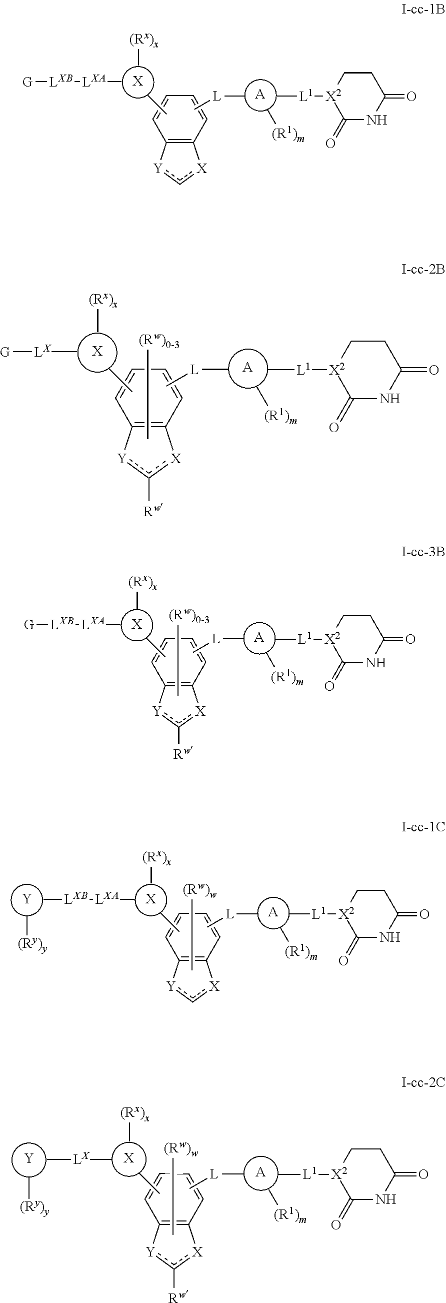 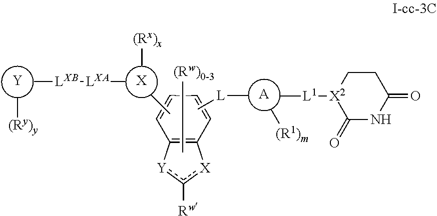
or a pharmaceutically acceptable salt thereof; or
the compound is of formula I-cc-1D, I-cc-2D, I-cc-3D, I-cc-1E, I-cc-2E, or I-cc-3E: 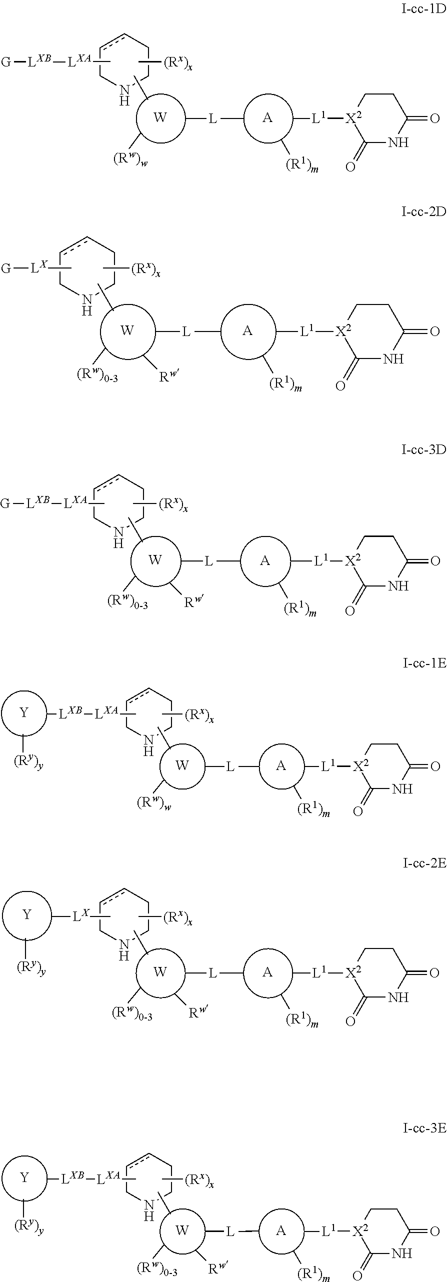
or a pharmaceutically acceptable salt thereof; or
the compound is of formula I-cc-1F, I-cc-2F, I-cc-3F, I-cc-1G, I-cc-2G, or I-cc-3G: 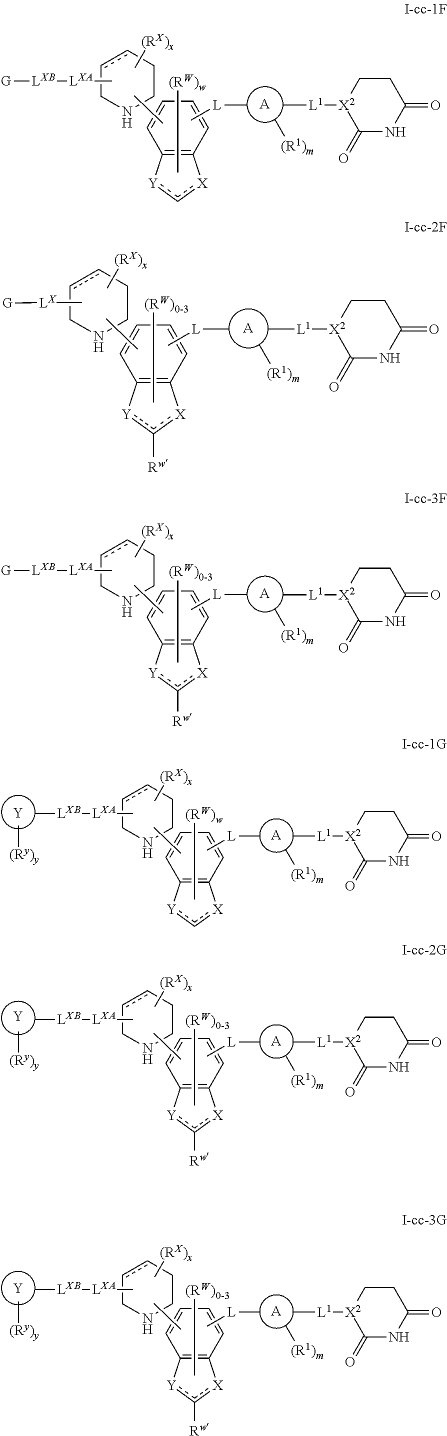
or a pharmaceutically acceptable salt thereof; or
the compound is of formula I-cc-1H, I-cc-2H, 1-cc-3H, I-cc-J I-cc-2J, or I-cc-3J: 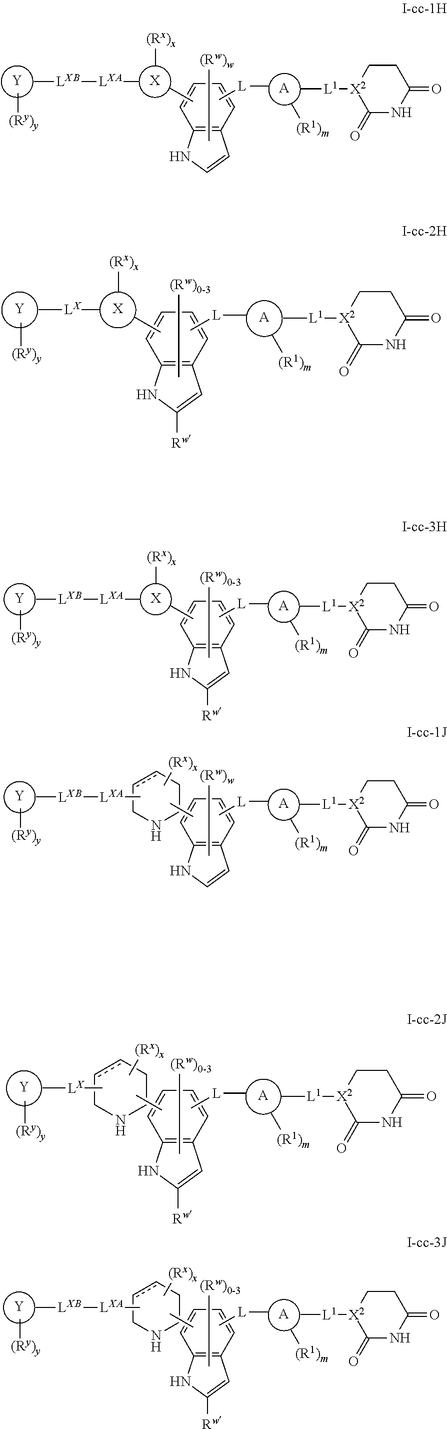
or a pharmaceutically acceptable salt thereof.

19. The compound of any one of claims 2-18, wherein, L is: 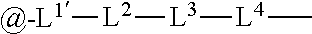
wherein:
@ represents the point of attachment to Ring W;
L^{1′} is a covalent bond or an optionally substituted bivalent, saturated or partially unsaturated, straight or branched C₁₋₆ hydrocarbon chain, wherein 1 methylene unit of L^{1′} is optionally replaced by -Cy^{L1}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—;
L² is a covalent bond or an optionally substituted bivalent, saturated or partially unsaturated, straight or branched C₁₋₆ hydrocarbon chain, wherein 1 methylene unit of L² is optionally replaced by -Cy^{L2}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—;
L³ is a covalent bond or an optionally substituted bivalent, saturated or partially unsaturated, straight or branched C₁₋₆ hydrocarbon chain, wherein 1 methylene unit of L³ is optionally replaced by -Cy^{L3}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—;
L⁴ is a covalent bond or an optionally substituted bivalent, saturated or partially unsaturated, straight or branched C₁₋₆ hydrocarbon chain, wherein 1 methylene unit of L⁴ is optionally replaced by -Cy^{L4}-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, or —NRC(O)O—; and
each of -Cy^{L1}-Cy^{L2}-, -Cy^{L3}- and -Cy^{L4}- is independently -Cy-.

20. The compound of claim 19, wherein L is: 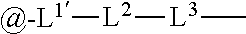
wherein:
L^{1′} is -Cy^{L1}-;
L² is -Cy^{L2}-; and
L³ is an optionally substituted bivalent, saturated or partially unsaturated, straight or branched C₁₋₆ hydrocarbon chain.

21. The compound of claim 19, wherein L is: 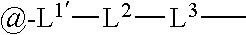
wherein:
L^{1′} is -Cy^{L1}-:
L² is -Cy^{L2}-; and
L³ is -Cy^{L3}-.

22. The compound of claim 19, wherein L is: 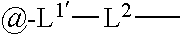
wherein:
@ represents the point of attachment to Ring W:
L^{1′} is -Cy^{L1}-; and
L² is -Cy^{L2}-.

23. The compound of claim 19, wherein:
the compound is of formula I-dd, I-dd-1, I-dd-2, I-dd-3, I-dd-A, I-dd-2A, I-dd-2A, or I-dd-3A: 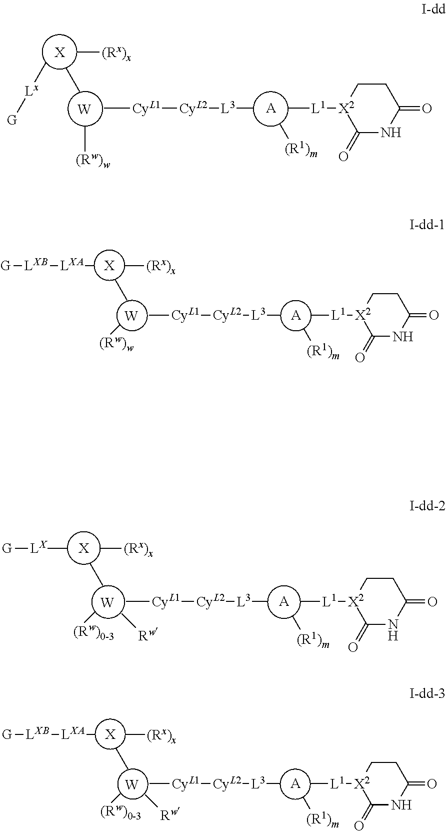 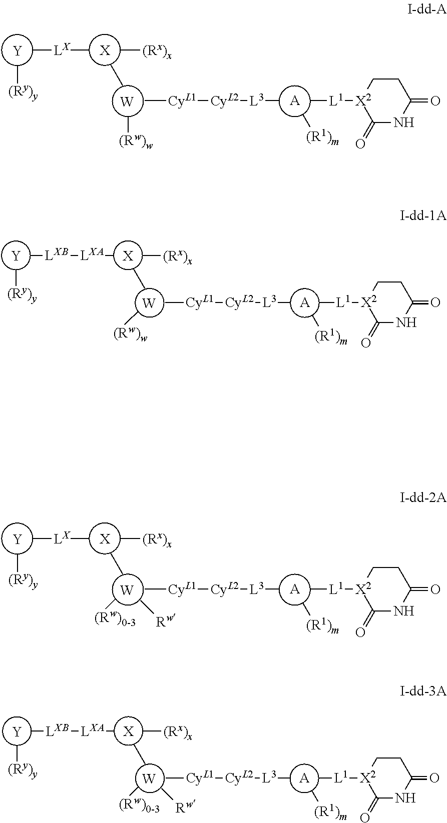
or a pharmaceutically acceptable salt thereof; or
the compound is of formula I-dd-B, I-dd-1B, I-dd-2B, I-dd-3B, I-dd-C, I-dd-2C, I-dd-2C, or I-dd-3C: 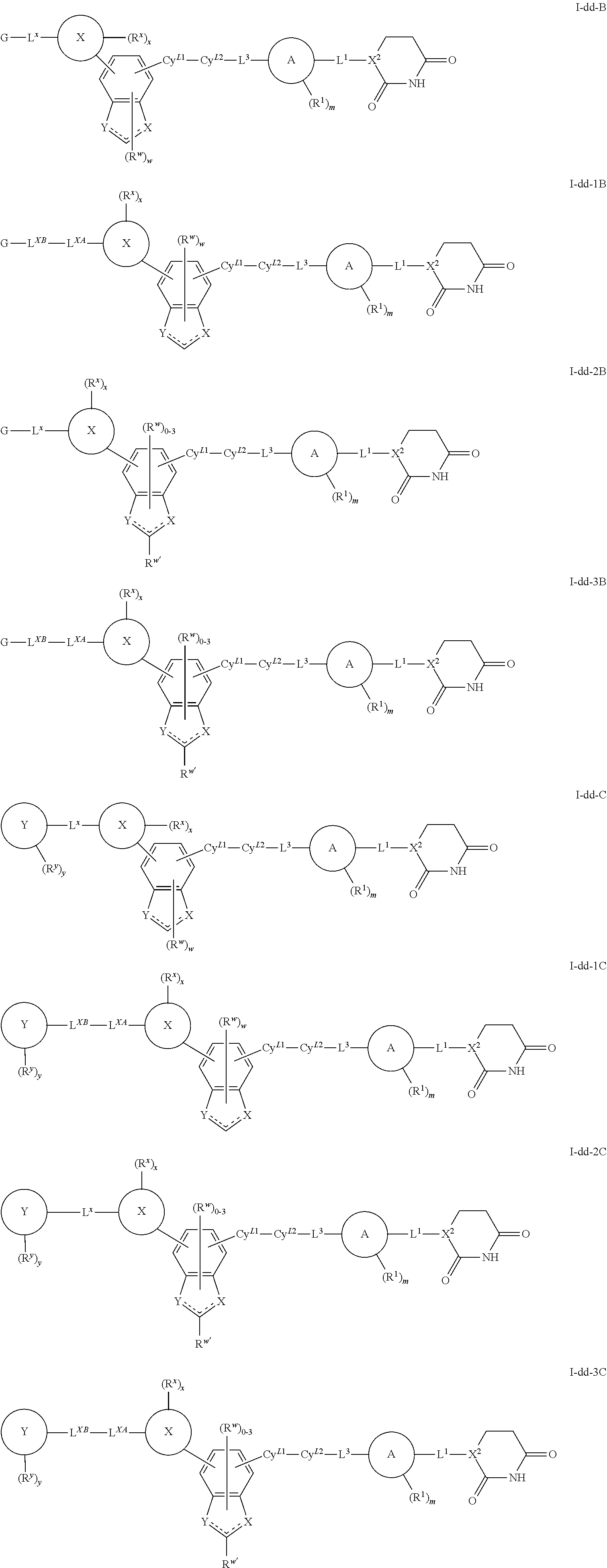
or a pharmaceutically acceptable salt thereof; or
the compound is of formula I-dd-D, I-dd-1D, I-dd-2D, I-dd-3D, I-dd-E, I-dd-2E, I-dd-2E, or I-dd-3E: 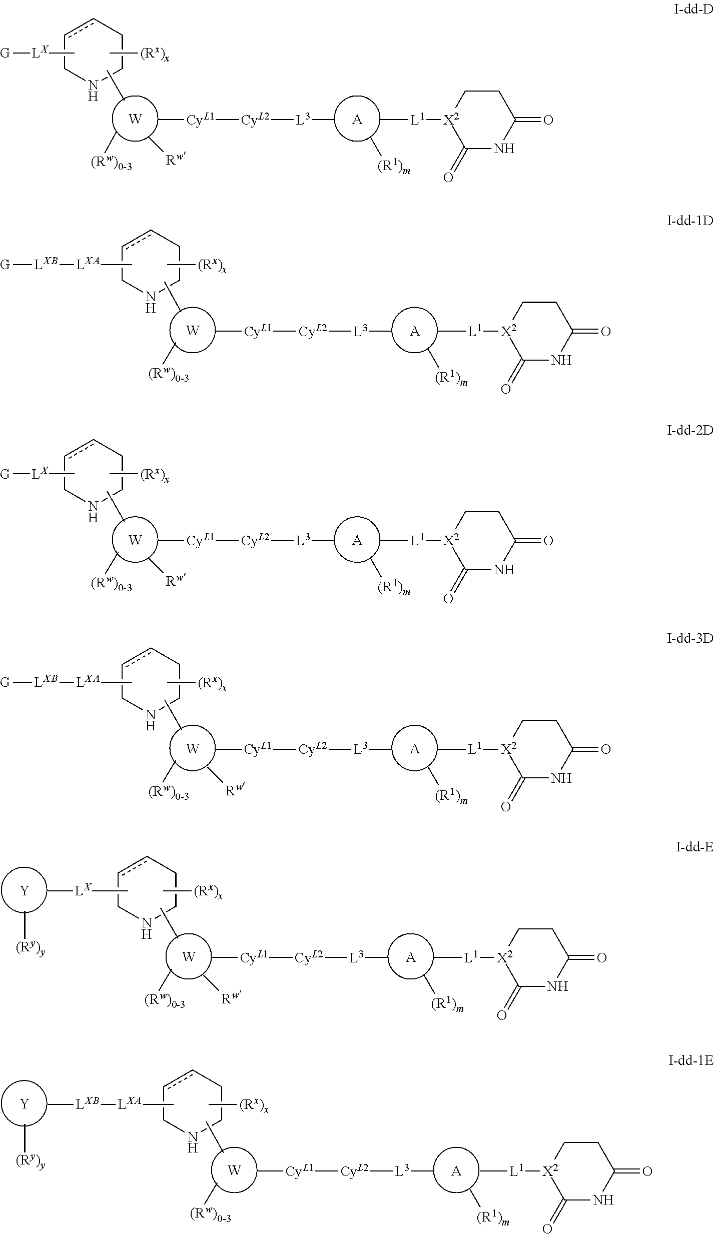 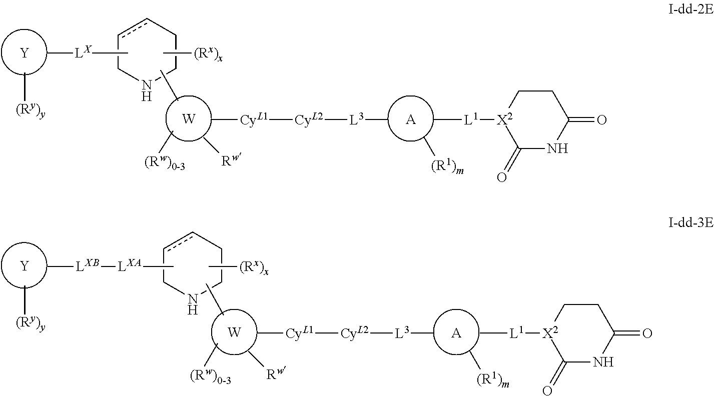
or a pharmaceutically acceptable salt thereof; or
the compound is of formula I-dd-F, I-dd-1F, I-dd-2F, I-dd-3F, I-dd-G, I-dd-2G, I-dd-2G, or I-dd-3G: 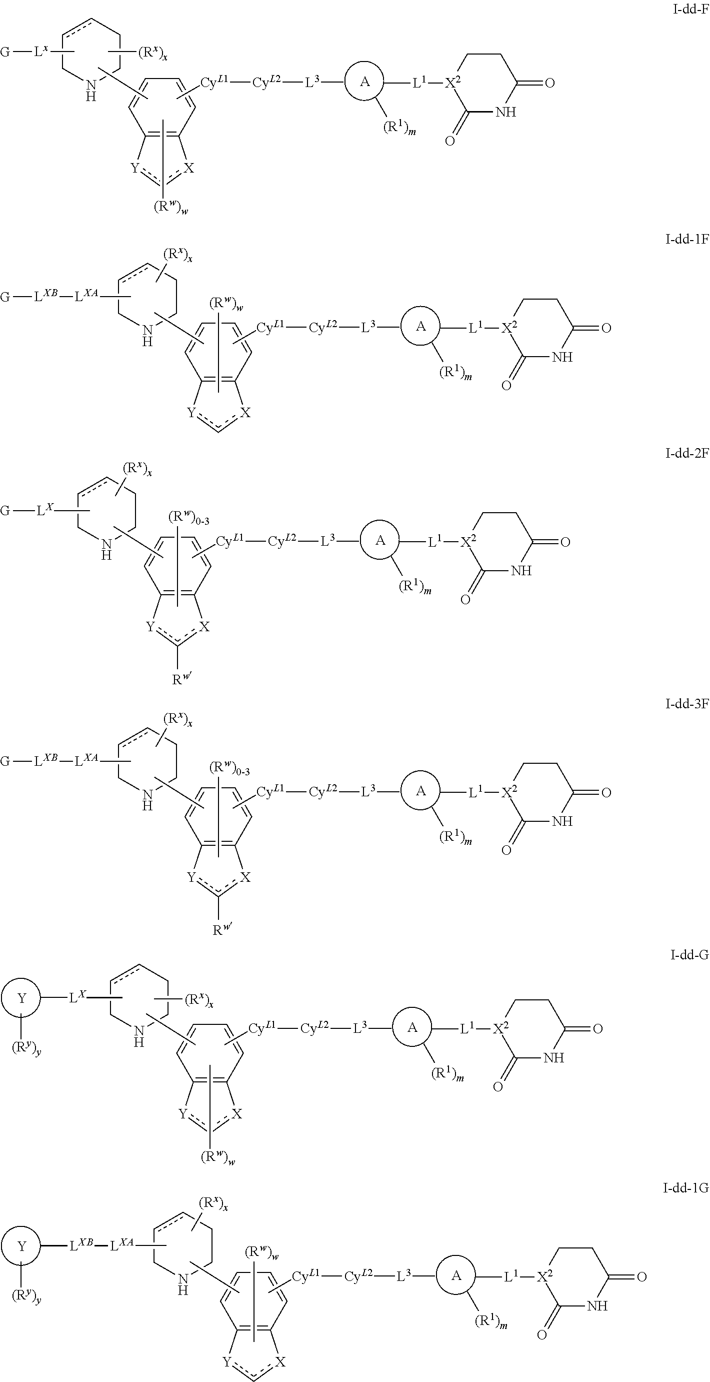 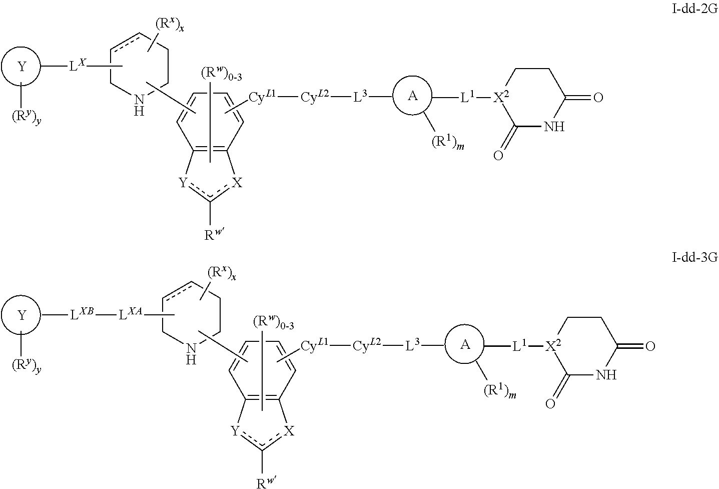
or a pharmaceutically acceptable salt thereof; or
the compound is of formula I-dd-H, I-dd-1H, I-dd-2H, I-dd-3H, I-dd-J, I-dd-2J, I-dd-2J, or I-dd-3J: 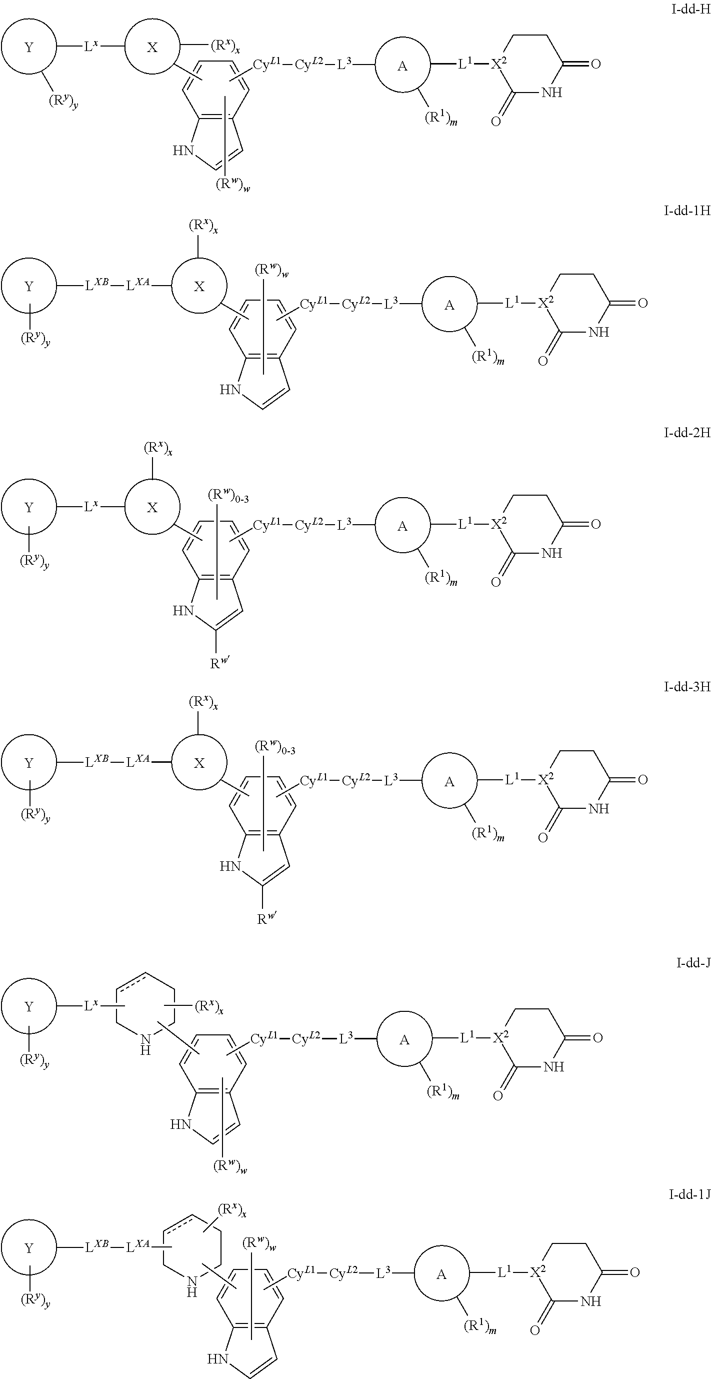 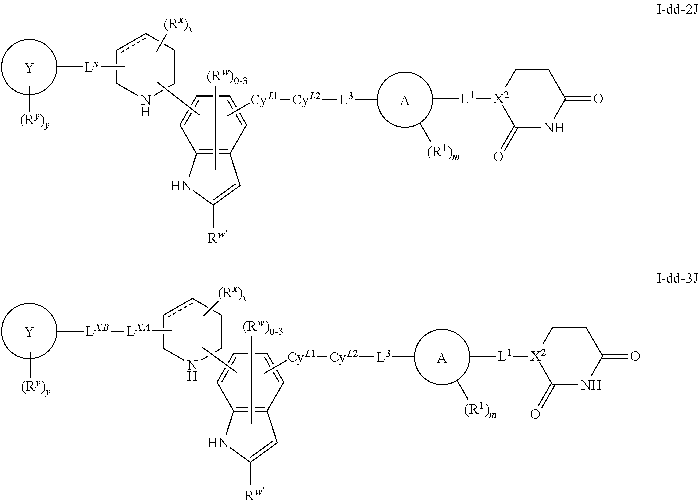
or a pharmaceutically acceptable salt thereof.

24. The compound of any one of claims 1-23, wherein:
Ring A is a ring selected from phenylenyl, naphthalenyl, pyridinylenyl, 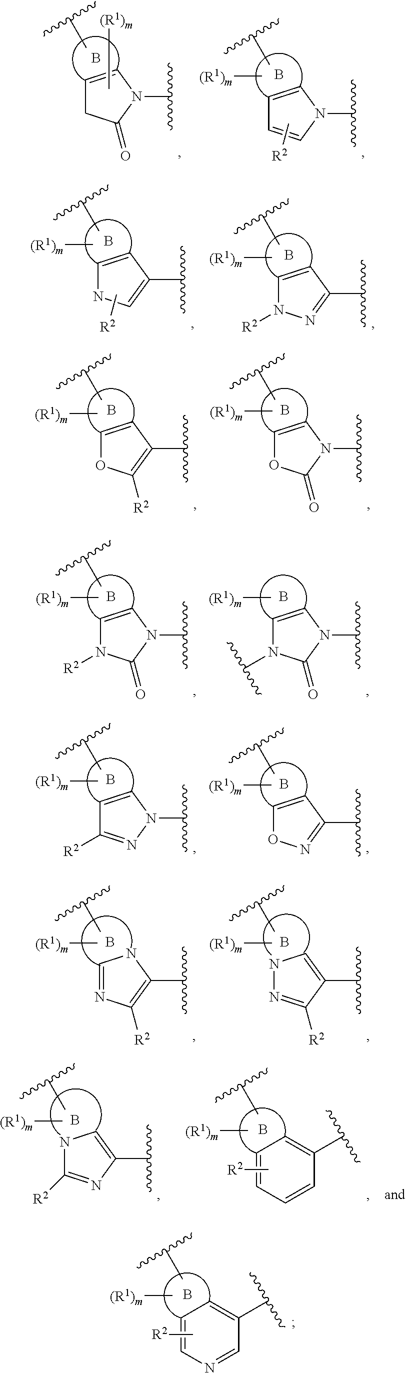
Ring B is a fused ring selected from benzo, a saturated or partially unsaturated 4-7 membered carbocyclyl, a saturated or partially unsaturated 4-7 membered heterocyclyl having 1-2 heteroatoms independently selected from nitrogen, oxygen, and sulfur, and a 5-6 membered heteroaryl containing 1-4 heteroatoms independently selected from nitrogen, oxygen, and sulfur:
each R¹ is independently R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)N(R)OR, —CR₂N(R)C(O)R, —CR₂N(R)C(O)NR₂, —CFR₂, —CF₂R, —CF₃, —CR₂(OR), —CR₂(NR₂), —OC(O)R, —OC(O)NR₂, —OP(O)R₂, —OP(O)(OR)₂, —OP(O)(OR)NR₂, —OP(O)(NR₂)₂, —N(R)C(O)OR, —N(R)C(O)R, —N(R)C(O)NR₂, —N(R)S(O)₂R, —N(R)P(O)R₂, —N(R)P(O)(OR)₂, —N(R)P(O)(OR)NR₂, —N(R)P(O)(NR₂)₂, or —N(R)S(O)₂R: or:
two R¹ groups of Ring A are taken together with their intervening atoms to form an optionally substituted ring selected from a 3-10 membered saturated or partially unsaturated carbocyclyl or heterocyclyl having 1-4 heteroatoms independently selected from nitrogen, oxygen, or sulfur; benzo; or a 3-10 membered heteroaryl having 1-4 heteroatoms independently selected from nitrogen, oxygen, or sulfur;
R² is hydrogen, halogen, C₁₋₆ alkyl, C₃₋₆cycloalkyl, C₁₋₆ haloalkyl, —OC₁₋₆ alkyl, —OC₃₋₆cycloalkyl, or —OC₁₋₆ haloalkyl; or:
an R² and an R¹ are taken together with their intervening atoms to form an optionally substituted ring selected from a 3-10 membered saturated or partially unsaturated carbocyclyl or heterocyclyl having 1-4 heteroatoms independently selected from nitrogen, oxygen, or sulfur; benzo; or a 5-10 membered heteroaryl having 1-4 heteroatoms independently selected from nitrogen, oxygen, or sulfur.

25. The compound of any one of claims 1-24, wherein:
the structure 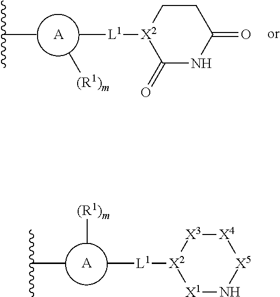
is of any one of formula I-aa-10′, I-aa-11′, I-aa-12′, I-aa-13′, or I-aa-14′: 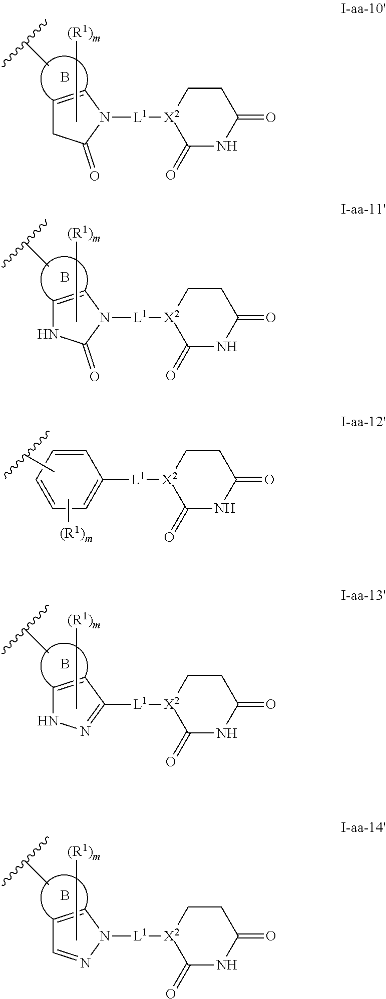
or a pharmaceutically acceptable salt thereof; or
the structure 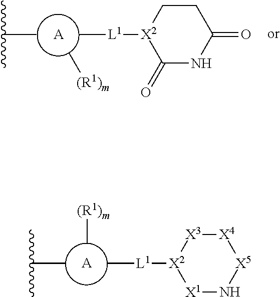
is of any one of formula I-aa-10a′, I-aa-11a′, I-aa-12a′, I-aa-12b′, I-aa-13a′, or I-aa-14a′: 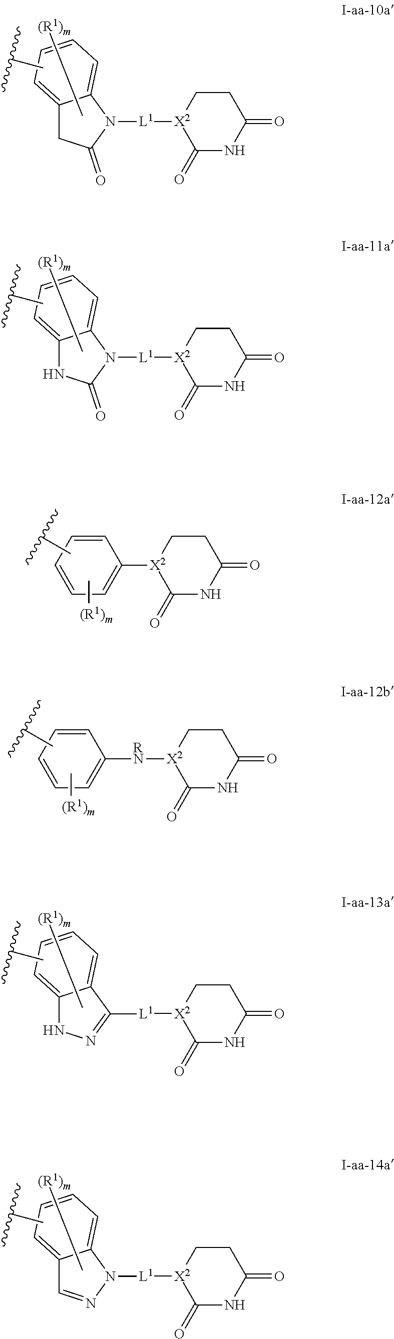
or a pharmaceutically acceptable salt thereof; or
the structure 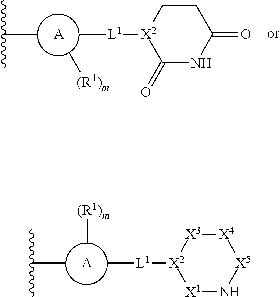
is of any one of formula I-aa-2′, I-aa-3′, or I-aa-4′: 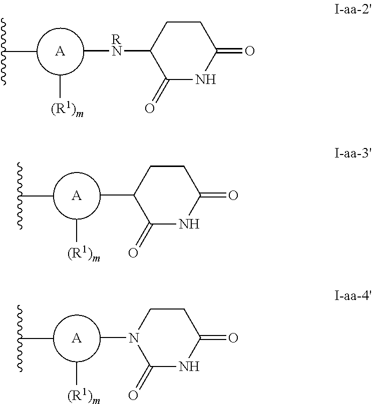
or a pharmaceutically acceptable salt thereof.

26. The compound of any one of claims 1-25, wherein:
R^{x} is selected from hydrogen, Ra, halogen, —CN, —NO₂, —OR, —SR, —NR₂, —SiR₃, —S(O)R, —S(O)₂R, —S(O)(NR)R—S(O)₂NR₂, —S(O)R, —C(O)R, —C(O)OR, —C(O)NR₂, —C(O)NROR, —OC(O)R, —OC(O)NR₂, —P(O)R₂, —P(O)(OR)₂, —OP(O)R₂, —OP(O)(OR)₂, —NRC(O)OR, —NRC(O)N(R)₂, and —NRS(O)₂R: or
each R¹ is independently R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)N(R)OR, —C(R)₂N(R)C(O)R, —C(R)₂N(R)C(O)NR₂, —OC(O)R, —OC(O)NR₂, —OP(O)R₂, —OP(O)(OR)₂, —OP(O)(OR)(NR₂), —OP(O)(NR₂)₂, —N(R)C(O)OR, —N(R)C(O)NR₂, —N(R)S(O)₂R, —NP(O)R₂, —N(R)P(O)(OR)₂, —N(R)P(O)(OR)(NR₂), —N(R)P(O)(NR₂)₂, —N(R)S(O)₂R; or: two R¹ groups on the same or adjacent atoms are taken together with their intervening atoms to form an optionally substituted ring selected from a 3-10 membered saturated or partially unsaturated carbocyclyl or heterocyclyl having 1-4 heteroatoms independently selected from nitrogen, oxygen, or sulfur; benzo; or a 5-10 membered heteroaryl having 1-4 heteroatoms independently selected from nitrogen, oxygen, or sulfur.

27. The compound of claim 1 or 2, wherein said compound is selected from any one of the compounds depicted in Table 1, or a pharmaceutically acceptable salt thereof.

28. A pharmaceutical composition comprising a compound of any one of claims 1-27, and a pharmaceutically acceptable carrier, adjuvant, or vehicle.

29. A method of degrading STAT6 in a patient or biological sample comprising administering to said patient, or contacting said biological sample with a compound of any one of claims 1-27, or a pharmaceutical composition thereof.

30. A method of treating an STAT6-mediated disorder, disease, or condition in a patient comprising administering to said patient a compound of any of one claims 1-27, or a pharmaceutical composition thereof.

31. The method of claim 30, wherein STAT6-mediated disorder, disease, or condition is cancer, a neurodegenerative disorder, a viral disease, an autoimmune disease, an inflammatory disorder, a hereditary disorder, a hormone-related disease, a metabolic disorder, conditions associated with organ transplantation, immunodeficiency disorders, a destructive or overgrowing bone disorder, a proliferative disorder, an infectious disease, a condition associated with cell death, thrombin-induced platelet aggregation, liver disease, pathologic immune conditions involving T cell activation, a cardiovascular disorder, or a CNS disorder.
